(19) United States
(12) Patent Application Publication
(10) Pub. No.: US 2024/0043912 A1
(43) Pub. Date: Feb. 8, 2024

(54) GENOMIC SELECTION (GS) BREEDING CHIP OF HUAXI CATTLE AND USE THEREOF

(71) Applicant: Institute of Animal Sciences of CAAS, Beijing (CN)

(72) Inventors: Yan CHEN, Beijing (CN); Junya LI, Beijing (CN); Han GAO, Beijing (CN); Fei GE, Beijing (CN); Huijiang GAO, Beijing (CN); Xue GAO, Beijing (CN); Lupei ZHANG, Beijing (CN); Lingyang XU, Beijing (CN); Bo ZHU, Beijing (CN); Zezhao WANG, Beijing (CN)

(21) Appl. No.: 18/224,725

(22) Filed: Jul. 21, 2023

(30) Foreign Application Priority Data
Jul. 27, 2022 (CN) 2022108894331

(51) Int. Cl. C12Q 1/6827; G16B 20/40; G16B 20/20; G16B 35/20; A01K 67/02

(52) U.S. Cl. CPC C12Q 1/6827; G16B 20/40; G16B 20/20; G16B 35/20; A01K 67/02

(57) ABSTRACT

The present disclosure provides a genomic selection (GS) breeding chip of Huaxi cattle and use thereof, and relates to the technical field of molecular breeding. The present disclosure provides a molecular marker combination for whole-genome genotyping of Huaxi cattle, where a genotyping object involves 112,177 single nucleotide polymorphisms (SNPs) and 3 Indels, including seven marker panels in total. In the present disclosure, a genome-wide breeding chip constructed with the molecular marker combination has functional relevance, pertinence and effectiveness, innovation, comprehensiveness, practicality, and cost-effectiveness. This breeding chip also shows uniform distribution of chromosomes in the whole-genome, high coverage, and moderate throughput of markers. This breeding chip has desirable compatibility with existing commercial chips and high cost performance.

### CROSS REFERENCE TO RELATED APPLICATION

This patent application claims the benefit and priority of Chinese Patent Application No. 2022108894331, filed with the China National Intellectual Property Administration on Jul. 27, 2022, the disclosure of which is incorporated by reference herein in its entirety as part of the present application.

### TECHNICAL FIELD

The present disclosure belongs to the technical field of molecular breeding, and in particular relates to a genomic selection (GS) breeding chip of Huaxi cattle and use thereof.

### BACKGROUND

Breed is a determinant of the quality and efficiency of livestock and poultry farming. Traditional breeding is mainly based on conventional breeding methods or processes such as appearance assessment, pedigree records, performance determination, and descendant determination. Compared with pigs, poultry and other high reproductive livestock and poultry, beef cattle have long generation interval and low reproductive rate. In beef cattle, most of the important economic traits are quantitative traits, and traits such as carcass and meat quality have low heritability, high cost of determination, and difficult in early determination. These factors make the conventional breeding to conduct breed improvement and new breed breeding of beef cattle relatively slowly. Genomic selection (GS), the main selection and breeding technology in current livestock and poultry breeding, can effectively shorten generation interval and accelerate genetic progress. This method estimates an individual's genomic breeding value with genetic markers covering the entire genome. Based on the marker information and phenotype information of known populations, the association between markers and phenotypes is established. In this way, the effects of all genotype markers can be estimated on a genome-wide scale, and then reasonable predictions can be made for populations with unknown phenotypes, thereby achieving more comprehensive and reliable selection of varieties. The GS has been applied to the breeding practice of dairy cattle, beef cattle, pigs, chickens and other livestock and poultry, improves the accuracy of animal breeding, and significantly reduces the cost of breeding.

Single nucleotide polymorphism (SNP) genotyping detection technology is mainly based on whole-genome resequencing and SNP genotyping beadchip. The whole-genome resequencing can obtain the most comprehensive genome variation information, but has cumbersome operation procedures and complex data analysis. If this technology is applied to large-scale analysis with a large number of individuals, there may be relatively high sequencing and computing costs. The gene beadchip technology used for SNP genotyping arranges millions of DNA marker sequences on glass slides, silicon wafers and other solid-phase media, and fixes them to form an SNP beadarray. The DNA probe sequences on the chip are complementary hybridized with a target genome, and the SNP is genotyped by fluorescence scanning. At present, there are many types of beadchips used for SNP genotyping in the field of beef cattle breeding. The two most commercialized SNP beadchip products used in Chinese beef cattle breeding are Illumina BovineHD (770K) and Neogen GGP Bovine 100K. However, the markers used in existing commercial chips are mainly derived from foreign beef cattle breeds, and the effective information only account for about 80%, while many SNPs fail to be detected. Moreover, these beadchips lack the genomic genetic variation markers of Chinese beef cattle breeds, and show a high cost. This limits the popularization and application of GS in beef cattle breeding in China.

Compared with foreign beef cattle breeds, the slaughter rate, meat production rate, and carcass weight of local cattle breeds in China are significantly different from those in foreign countries with developed beef cattle industries. Huaxi cattle, as a new breed of large-scale specialized beef cattle that has been independently bred in China for more than 40 years, passed the examination and approval of the National Animal Genetic Resources Committee in 2021. The Huaxi cattle has gone through three breeding stages: hybridization exploration stage (1978-1993), germplasm innovation stage (1994-2003), and breeding improvement (2004-present). This breed has fast growth, high slaughter rate and net meat rate, and wide adaptability and distribution. Currently, the total stock of Huaxi cattle exceeds heads, and the core herd contains more than 3,600 heads, mainly distributed in Inner Mongolia, Henan, Hubei, Jilin, Yunnan, and Xinjiang. In order to promote the continuous breeding and variety promotion of Huaxi cattle, as well as improve the production performance and self-supplying ability of Chinese beef cattle, it is of great significance to design a genome-wide genotyping chip for GS breeding of the Huaxi cattle. This chip is intended to improve an accuracy of a genomic estimated breeding value (GEBV) for important economic traits of the Huaxi cattle, accelerate genetic progress, shorten generation intervals, launch early selection, and reduce breeding costs.

### SUMMARY

In view of this, an objective of the present disclosure is to provide a GS breeding chip of Huaxi cattle and use thereof. The breeding chip is evenly distributed on chromosomes of a whole-genome, and has high coverage, moderate throughput of sites, high cost performance, and desirable compatibility with existing commercial beadchips.

To achieve the above objective, the present disclosure provides the following technical solutions:

The present disclosure provides a molecular marker combination for whole-genome genotyping of Huaxi cattle, including seven marker panels; where a first marker panel includes 7,221 SNPs; a second marker panel includes 22,937 SNPs and 1 Indel; a third marker panel includes 249 SNPs; a fourth marker panel includes 3,907 SNPs and 2 Indels; a fifth marker panel includes 6,190 SNPs; a sixth marker panel includes a total of 74,098 markers verified in a Huaxi cattle population from Illumina BovineHD chip and Neogen GGP Bovine 100K chip; and a seventh marker panel includes 2,617 SNPs.

The present disclosure further provides a whole-genome breeding chip of Huaxi cattle, including the molecular marker combination.

The present disclosure further provides use of the molecular marker combination or the genome-wide breeding chip in detection for genotyping of the Huaxi cattle.

The present disclosure further provides use of the molecular marker combination or the genome-wide breeding chip in genome-wide association study (GWAS) of the Huaxi cattle.

The present disclosure further provides use of the molecular marker combination or the genome-wide breeding chip in parentage testing of the Huaxi cattle.

The present disclosure further provides use of the molecular marker combination or the genome-wide breeding chip in GS breeding of the Huaxi cattle.

### Beneficial Effects

the present disclosure provides a molecular marker combination for whole-genome genotyping of Huaxi cattle, where genotyping objects involve 112,177 SNPs and 3 Indel sites, including seven marker panels. A first marker panel has a total of 7,221 SNPs associated with important economic traits of Huaxi cattle, including 3,065 SNPs obtained from GWAS analysis of important economic traits such as growth and development traits, fattening traits, carcass traits, meat quality traits, and reproductive traits, as well as 4,158 SNPs obtained by BayesB analysis on 11 important economic traits. A second marker panel has a set of functional region SNPs and Indels that integrate genome resequencing, transcriptome, exome, epigenome and other omics data, with a total of 22,938 markers including 3,800 SNPs related to three tissue-specific gene expressions of longissimus dorsi muscle, liver, and subcutaneous fat, 9,242 SNPs in the exon region related to the difference between high and low phenotypes screened by exon capture sequencing, 1 Indel, and 9,985 SNPs involved in epigenetic and gene regulation screened by ATAC-seq (assay for transposase accessible chromatin with high-throughput sequencing). A third marker panel has 249 SNPs for identification of genetic relationship. A fourth marker panel has 3,907 functional SNPs and 2 Indels related to important economic traits and diseases of beef cattle, with a total of 3,909 markers. A fifth marker panel has a total of 6,190 SNPs related to important economic traits of beef cattle reported in the QTL database. A sixth marker panel has a total of 74,098 effective SNPs verified in the Huaxi cattle population by Illumina BovineHD beadchip and Neogen GGP Bovine 100K beadchip. A seventh marker panel has a total of 2,617 SNPs filling a gap region larger than 100 kb. The mentioned-above seven marker panels have a total of 112,177 SNPs and 3 Indels for the synthesis of probes and the customization of liquid-phase chips for genotyping by target sequencing.

In the present disclosure, the whole-genome breeding chip also has the following five features. A first is functional correlation: the trait-associated markers of Huaxi cattle selected involve seven categories of growth, fattening, carcass, meat quality, reproduction, disease, and health, with a total of 129 traits, including 13,581 important functional SNPs. Applying these SNPs to GS breeding can ensure the accuracy of GEBV estimation so as to accelerate the GS breeding process of Huaxi cattle. A second is pertinence and effectiveness: the selected markers come from the research results and data accumulation of population genetic evaluation and trait mining/utilization of Huaxi cattle. The functional markers have been verified by high-density beadchips and re-sequencing, and a polymorphism of the SNPs is excellent in the population, showing desirable validity. A third is innovation: through the optimal design of multi-level omics such as re-sequencing, transcriptome, exome, and epigenome, the newly discovered trait-specific functional SNPs and Indels of Huaxi cattle are selected. These markers are not recorded in the existing Ensemble database. A fourth is comprehensiveness: GEBV comprehensive evaluation is conducted on slaughter traits, meat quality traits, body size traits, and five traits used by genomic China beef index (GCBI) with high accuracy. A fifth is practicality and cost performance the breeding chip shows uniform distribution of chromosomes in the whole-genome, high coverage, and moderate throughput of markers. This breeding chip has desirable compatibility with existing commercial beadchips and high cost performance.

### BRIEF DESCRIPTION OF THE DRAWINGS

FIG. 1 shows a site distribution density of a Cattle110K chip in an example on a whole-genome;

FIG. 2 shows minimum allele frequency distribution of the Cattle110K chip markers in an example;

FIG. 3 shows distribution of different biological function units of the Cattle110K chip markers in an example;

FIG. 4 shows a phylogenetic tree of Huaxi cattle and closely related cattle breeds (populations); and

FIGS. 5A-F show Manhattan plot of GWAS based on Cattle110K genotyping results.

### DETAILED DESCRIPTION OF THE EMBODIMENTS

The present disclosure provides a molecular marker combination for whole-genome genotyping of Huaxi cattle, including 112,177 SNPs and 3 Indels, where genome position information of the marker loci is shown in SEQ ID NO: 1 to SEQ ID NO: 112180.

In the present disclosure, the whole-genome breeding chip is preferably a liquid-phase chip, and detection of the molecular markers can be genotyping based on targeted capture sequencing technology. The ARS-UCD 1.2/bosTau9 version is preferably used as a reference genome of cattle. In the examples, the genome-wide breeding chip of Huaxi cattle is named “Cattle110K” chip. The working principle is: based on a DNA extracted from the tested blood sample, targeted capture and sequencing are conducted through base pairing, so as to realize the gene detection and genotyping of a target region. With the “Cattle110K” chip, the DNA samples of Huaxi cattle can be detected, such that this chip is applied to the whole-genome of Huaxi cattle for SNP genotyping, genomic selection, GWAS, population genetics analysis, gene fine mapping, genome-wide linkage study, parentage testing, and germplasm evaluation.

The present disclosure further provides use of the molecular marker combination or the genome-wide breeding chip in genome-wide association study (GWAS) of the Huaxi cattle. In the present disclosure, the use is preferably the same as the above content, and is not repeated here.

The present disclosure further provides use of the molecular marker combination or the genome-wide breeding chip in parentage testing of the Huaxi cattle. In the present disclosure, the use is preferably the same as the above content, and is not repeated here.

The present disclosure further provides use of the molecular marker combination or the genome-wide breeding chip in GS breeding of the Huaxi cattle. In the present disclosure, the use is preferably the same as the above content, and is not repeated here.

The GS breeding chip of Huaxi cattle and the use thereof provided by the present disclosure are described in detail below with reference to the examples, but these examples should not be understood as limiting the claimed scope of the present disclosure.

### Example 1

Acquisition of Seven Marker Panels for Cattle110K Huaxi Cattle Genome-Wide SNP Chip

In the present disclosure, the molecular marker combination involved 7 panels in total.

In the present disclosure, a method for obtaining a first panel of markers preferably included: 1,233 individuals with the most complete phenotype data records were selected among 4,694 individuals genotyped with the Illumina BovineHD (770K) beadchip. Taking the whole-genome resequencing data of 44 Huaxi cattle individuals as a reference group, the BovineHD results of 1,233 individuals were imputed into the level of resequencing genotype data. SNPs with MAF less than 0.05, individual deletion rate greater than 10%, and without HWE (hardy-weinberg equilibrium) P value of 1×10⁻⁶ were eliminated, and finally a total of 6,776,719 SNPs were remained for screening SNPs closely related to important economic traits of Huaxi cattle. Preferably, there were two parts: a first part was to conduct GWAS on important economic traits such as growth and development traits, fattening traits, carcass traits, meat quality traits, and reproductive traits. Specifically, the traits included growth and development traits (birth weight, weaning weight, yearling weight), fattening traits (daily gain during fattening period), carcass weight traits (carcass weight, lean meat weight, bone weight), carcass meat production traits (dressing percentage, lean meat percentage, carcass meat percentage), carcass morphological traits (carcass length, carcass depth, hind leg depth of carcass), meat quality traits (marbling, tenderness, pH value), and reproductive traits (calving ease). A total of 3,065 SNPs (P<5×10⁻⁸) significantly associated with traits were obtained after deduplication. A second part was to conduct BayesB analysis on a total of 11 important economic traits, including weaning weight, daily gain during fattening period, slaughter weight, carcass weight, chuck roll weight, ribeye weight, retail meat weight, dressing percentage, lean meat percentage, marbling score, and shear force. Sorting was done according to SNP effect value, and the top 0.01% of loci were selected. After deduplication, a total of 4,158 large-effect SNPs were obtained. Based on the combination of the above trait-associated analysis results, a total of 7,221 functional SNPs of important economic traits of Huaxi cattle were obtained through biostatistical analysis and deduplication. The information of first marker panel was shown in Table 1:

TABLE 1
Location information of first marker panel

| NO. | Position | NO. | Position | NO. | Position | NO. | Position | NO. | Position |
|---|---|---|---|---|---|---|---|---|---|
| 27 | 1:1073001 | 40 | 1:1271108 | 124 | 1:3014460 | 164 | 1:3598559 | 213 | 1:4312806 |
| 216 | 1:4341656 | 257 | 1:5479286 | 286 | 1:6042789 | 314 | 1:6638827 | 324 | 1:6809931 |
| 386 | 1:8222113 | 399 | 1:8645362 | 407 | 1:8747963 | 480 | 1:10381445 | 484 | 1:10442662 |
| 547 | 1:12065998 | 575 | 1:12850062 | 586 | 1:13291614 | 593 | 1:13478803 | 594 | 1:13478804 |
| 595 | 1:13478805 | 600 | 1:13618910 | 602 | 1:13649839 | 603 | 1:13650350 | 604 | 1:13650573 |
| 605 | 1:13650578 | 606 | 1:13657524 | 607 | 1:13657759 | 608 | 1:13657853 | 609 | 1:13659104 |
| 610 | 1:13659735 | 611 | 1:13660313 | 612 | 1:13662505 | 613 | 1:13662879 | 614 | 1:13663313 |
| 615 | 1:13663369 | 632 | 1:14229816 | 635 | 1:14367624 | 640 | 1:14444223 | 646 | 1:14671195 |
| 675 | 1:15289219 | 732 | 1:17104389 | 745 | 1:17433162 | 761 | 1:18058788 | 779 | 1:18612790 |
| 803 | 1:19491923 | 807 | 1:19546032 | 831 | 1:20249479 | 855 | 1:20731028 | 866 | 1:21118209 |
| 901 | 1:22054771 | 936 | 1:23011477 | 954 | 1:23715328 | 957 | 1:23772692 | 974 | 1:24145046 |
| 1014 | 1:25285394 | 1015 | 1:25305166 | 1018 | 1:25448644 | 1038 | 1:25974291 | 1075 | 1:26927446 |
| 1089 | 1:27357800 | 1132 | 1:29040826 | 1142 | 1:29268481 | 1173 | 1:30001509 | 1176 | 1:30051285 |
| 1177 | 1:30052889 | 1206 | 1:31087002 | 1207 | 1:31090461 | 1211 | 1:31197827 | 1243 | 1:32461661 |
| 1255 | 1:32885018 | 1263 | 1:33160247 | 1384 | 1:36327051 | 1385 | 1:36335560 | 1404 | 1:36719216 |
| 1410 | 1:36920529 | 1412 | 1:36984896 | 1458 | 1:38181926 | 1480 | 1:38770057 | 1483 | 1:38899433 |
| 1495 | 1:39263861 | 1539 | 1:40637359 | 1546 | 1:40754498 | 1571 | 1:41666745 | 1617 | 1:42491489 |
| 1667 | 1:43750606 | 1687 | 1:44125197 | 1691 | 1:44260960 | 1710 | 1:44679137 | 1713 | 1:44805494 |
| 1719 | 1:44925165 | 1720 | 1:44925173 | 1721 | 1:44925749 | 1722 | 1:44927653 | 1723 | 1:44928339 |
| 1724 | 1:44928350 | 1725 | 1:44928489 | 1726 | 1:44929514 | 1727 | 1:44929625 | 1728 | 1:44929973 |
| 1729 | 1:44931081 | 1730 | 1:44931463 | 1753 | 1:45241871 | 1820 | 1:47384886 | 1828 | 1:47617677 |
| 1841 | 1:48116097 | 1963 | 1:52181284 | 1995 | 1:53016532 | 2003 | 1:53237486 | 2005 | 1:53252387 |
| 2006 | 1:53260493 | 2009 | 1:53326573 | 2013 | 1:53415161 | 2016 | 1:53539734 | 2030 | 1:53652726 |
| 2044 | 1:53770698 | 2051 | 1:54027097 | 2052 | 1:54039879 | 2053 | 1:54067827 | 2054 | 1:54068465 |
| 2062 | 1:54170560 | 2063 | 1:54175315 | 2073 | 1:54409914 | 2077 | 1:54504340 | 2078 | 1:54507288 |
| 2094 | 1:54985100 | 2100 | 1:55132890 | 2113 | 1:55602206 | 2115 | 1:55658614 | 2130 | 1:56173438 |
| 2133 | 1:56288769 | 2135 | 1:56366295 | 2136 | 1:56367389 | 2137 | 1:56367471 | 2140 | 1:56429824 |
| 2141 | 1:56439077 | 2151 | 1:56684792 | 2155 | 1:56767210 | 2164 | 1:56950851 | 2239 | 1:58810751 |
| 2254 | 1:59120478 | 2289 | 1:60052451 | 2299 | 1:60321205 | 2305 | 1:60534555 | 2314 | 1:60643904 |
| 2322 | 1:60871605 | 2331 | 1:61098681 | 2344 | 1:61320487 | 2345 | 1:61335565 | 2350 | 1:61476653 |
| 2374 | 1:62211191 | 2410 | 1:62916376 | 2411 | 1:62918132 | 2415 | 1:63017753 | 2416 | 1:63029817 |
| 2417 | 1:63033101 | 2418 | 1:63035068 | 2419 | 1:63037531 | 2420 | 1:63038112 | 2421 | 1:63038183 |
| 2422 | 1:63050131 | 2424 | 1:63119198 | 2425 | 1:63125457 | 2426 | 1:63128635 | 2427 | 1:63130293 |
| 2436 | 1:63393320 | 2450 | 1:63809663 | 2531 | 1:65592899 | 2574 | 1:66873010 | 2577 | 1:67103986 |
| 2584 | 1:67236736 | 2585 | 1:67260968 | 2636 | 1:68655539 | 2642 | 1:68743594 | 2664 | 1:68997540 |
| 2707 | 1:69843446 | 2722 | 1:70196536 | 2764 | 1:70655375 | 2776 | 1:70889623 | 2777 | 1:70925120 |
| 2811 | 1:71906639 | 2821 | 1:72100009 | 2896 | 1:73841520 | 2929 | 1:74779617 | 2950 | 1:75412538 |
| 2963 | 1:75770166 | 2964 | 1:75770197 | 2965 | 1:75770199 | 2966 | 1:75783854 | 2971 | 1:75829393 |
| 2984 | 1:76114883 | 3005 | 1:76860758 | 3042 | 1:77680697 | 3045 | 1:77701368 | 3070 | 1:78334604 |
| 3071 | 1:78335278 | 3075 | 1:78397021 | 3113 | 1:79354013 | 3224 | 1:81621771 | 3225 | 1:81658300 |
| 3262 | 1:82505239 | 3290 | 1:83265388 | 3301 | 1:83612627 | 3349 | 1:85328309 | 3362 | 1:85790627 |
| 3458 | 1:88779015 | 3466 | 1:88853263 | 3483 | 1:89288724 | 3506 | 1:89791299 | 3511 | 1:89828935 |
| 3523 | 1:90054999 | 3528 | 1:90169751 | 3539 | 1:90383988 | 3572 | 1:91171394 | 3584 | 1:91442961 |
| 3603 | 1:91945075 | 3608 | 1:92172948 | 3613 | 1:92213222 | 3618 | 1:92387065 | 3635 | 1:93023669 |
| 3663 | 1:93808595 | 3690 | 1:94443181 | 3742 | 1:95761493 | 3774 | 1:96387293 | 3818 | 1:97495906 |
| 3831 | 1:97875563 | 3835 | 1:97919421 | 3848 | 1:98319004 | 4084 | 1:105587286 | 4086 | 1:105625485 |
| 4088 | 1:105653161 | 4093 | 1:105729523 | 4163 | 1:107822647 | 4184 | 1:108388088 | 4193 | 1:108564048 |
| 4221 | 1:109261833 | 4235 | 1:109562630 | 4257 | 1:110017436 | 4317 | 1:111454247 | 4321 | 1:111612080 |
| 4346 | 1:112243802 | 4358 | 1:112481363 | 4360 | 1:112532754 | 4385 | 1:112920549 | 4398 | 1:113309692 |
| 4428 | 1:113860732 | 4469 | 1:114867292 | 4471 | 1:114870124 | 4500 | 1:115756884 | 4501 | 1:115772405 |
| 4512 | 1:116396361 | 4572 | 1:117632176 | 4674 | 1:119359964 | 4675 | 1:119363741 | 4707 | 1:120254924 |
| 4715 | 1:120408319 | 4716 | 1:120409192 | 4727 | 1:120576333 | 4737 | 1:120851703 | 4754 | 1:121481221 |
| 4758 | 1:121544917 | 4856 | 1:124230559 | 4872 | 1:124533841 | 4873 | 1:124538558 | 4889 | 1:124936627 |
| 4930 | 1:125655889 | 4933 | 1:125689194 | 4936 | 1:125738236 | 4941 | 1:125837099 | 4945 | 1:125909016 |
| 4952 | 1:126088394 | 5002 | 1:127555264 | 5013 | 1:127861573 | 5035 | 1:128339939 | 5048 | 1:128645880 |
| 5056 | 1:128748369 | 5218 | 1:131754936 | 5233 | 1:132161523 | 5273 | 1:133625347 | 5304 | 1:134472909 |
| 5333 | 1:135062430 | 5365 | 1:135657617 | 5404 | 1:135977700 | 5417 | 1:136372646 | 5458 | 1:137985176 |
| 5493 | 1:139072937 | 5525 | 1:140006988 | 5547 | 1:140601043 | 5559 | 1:140911883 | 5568 | 1:141070658 |
| 5577 | 1:141182724 | 5635 | 1:142058648 | 5639 | 1:142168313 | 5683 | 1:142908923 | 5952 | 1:148034123 |
| 5953 | 1:148040606 | 5954 | 1:148040677 | 5955 | 1:148044952 | 5956 | 1:148046284 | 5957 | 1:148046462 |
| 5958 | 1:148047932 | 5959 | 1:148053077 | 5960 | 1:148055822 | 5961 | 1:148058260 | 5962 | 1:148073345 |
| 5963 | 1:148073444 | 5964 | 1:148075396 | 5974 | 1:148329401 | 5980 | 1:148487864 | 6038 | 1:149409627 |
| 6039 | 1:149411937 | 6040 | 1:149413628 | 6045 | 1:149560052 | 6060 | 1:149819819 | 6139 | 1:151376946 |
| 6140 | 1:151406584 | 6141 | 1:151407059 | 6142 | 1:151407604 | 6152 | 1:151612724 | 6169 | 1:151767739 |
| 6171 | 1:151799413 | 6172 | 1:151799855 | 6173 | 1:151799970 | 6174 | 1:151800242 | 6175 | 1:151801164 |
| 6176 | 1:151801587 | 6177 | 1:151801737 | 6178 | 1:151801752 | 6191 | 1:152091262 | 6201 | 1:152265191 |
| 6202 | 1:152266056 | 6203 | 1:152266869 | 6204 | 1:152267613 | 6205 | 1:152267787 | 6206 | 1:152268300 |
| 6207 | 1:152268458 | 6213 | 1:152339615 | 6214 | 1:152343252 | 6216 | 1:152348515 | 6217 | 1:152349140 |
| 6219 | 1:152350298 | 6220 | 1:152351423 | 6221 | 1:152353474 | 6222 | 1:152353711 | 6223 | 1:152353915 |
| 6224 | 1:152354049 | 6225 | 1:152354217 | 6226 | 1:152354593 | 6227 | 1:152354711 | 6228 | 1:152355277 |
| 6229 | 1:152355296 | 6230 | 1:152355677 | 6231 | 1:152363064 | 6236 | 1:152422417 | 6266 | 1:152974350 |
| 6287 | 1:153459144 | 6356 | 1:154782431 | 6390 | 1:155509473 | 6395 | 1:155617067 | 6397 | 1:155639845 |
| 6398 | 1:155669488 | 6406 | 1:155709356 | 6410 | 1:155739527 | 6415 | 1:155837092 | 6420 | 1:155936300 |
| 6426 | 1:156024716 | 6437 | 1:156119189 | 6463 | 1:156640964 | 6466 | 1:156695097 | 6470 | 1:156760308 |
| 6486 | 1:157055516 | 6504 | 1:157454125 | 6507 | 1:157551305 | 6553 | 2:894974 | 6604 | 2:1577280 |
| 6679 | 2:3866542 | 6686 | 2:3991289 | 6688 | 2:4033904 | 6690 | 2:4040966 | 6691 | 2:4053609 |
| 6707 | 2:4311756 | 6747 | 2:5066365 | 6793 | 2:5931885 | 6795 | 2:5954528 | 6847 | 2:6974699 |
| 6858 | 2:7193712 | 6872 | 2:7370181 | 6879 | 2:7526996 | 6881 | 2:7574742 | 6942 | 2:9325130 |
| 6974 | 2:10332348 | 7018 | 2:11668736 | 7023 | 2:11736825 | 7056 | 2:13064383 | 7074 | 2:13460557 |
| 7080 | 2:13648131 | 7107 | 2:14339353 | 7122 | 2:14701151 | 7166 | 2:15712399 | 7233 | 2:17570676 |
| 7234 | 2:17577794 | 7308 | 2:18634499 | 7318 | 2:18907347 | 7319 | 2:18942177 | 7320 | 2:18943474 |
| 7321 | 2:18951656 | 7322 | 2:18958579 | 7323 | 2:18964610 | 7324 | 2:18967934 | 7333 | 2:19110921 |
| 7382 | 2:20297370 | 7385 | 2:20378241 | 7392 | 2:20658754 | 7396 | 2:20683165 | 7424 | 2:21454390 |
| 7453 | 2:22063850 | 7454 | 2:22086957 | 7457 | 2:22138573 | 7515 | 2:23573584 | 7538 | 2:24013539 |
| 7549 | 2:24246589 | 7565 | 2:24496758 | 7566 | 2:24500311 | 7567 | 2:24524614 | 7570 | 2:24563421 |
| 7588 | 2:24952433 | 7603 | 2:25454747 | 7645 | 2:26254049 | 7656 | 2:26496144 | 7725 | 2:27887225 |
| 7734 | 2:28111908 | 7757 | 2:28481427 | 7809 | 2:29740566 | 7881 | 2:31547829 | 7957 | 2:33826799 |
| 8010 | 2:35264773 | 8019 | 2:35614289 | 8041 | 2:36281226 | 8076 | 2:37317121 | 8084 | 2:37618680 |
| 8086 | 2:37625413 | 8229 | 2:41341923 | 8233 | 2:41443343 | 8234 | 2:41451445 | 8235 | 2:41463995 |
| 8236 | 2:41476600 | 8239 | 2:41525245 | 8240 | 2:41526634 | 8241 | 2:41546403 | 8242 | 2:41555839 |
| 8243 | 2:41555881 | 8244 | 2:41556165 | 8248 | 2:41609088 | 8249 | 2:41648397 | 8263 | 2:42040167 |
| 8264 | 2:42120013 | 8283 | 2:42489642 | 8284 | 2:42493609 | 8285 | 2:42495164 | 8313 | 2:43227689 |
| 8369 | 2:44933199 | 8396 | 2:45615535 | 8398 | 2:45632178 | 8409 | 2:45890270 | 8416 | 2:46041603 |
| 8493 | 2:47767849 | 8507 | 2:48158383 | 8511 | 2:48296454 | 8537 | 2:48966694 | 8542 | 2:48995422 |
| 8546 | 2:49074113 | 8587 | 2:50450300 | 8631 | 2:51841153 | 8632 | 2:51841281 | 8633 | 2:51845196 |
| 8634 | 2:51846388 | 8635 | 2:51848506 | 8636 | 2:51849168 | 8637 | 2:51850817 | 8638 | 2:51851321 |
| 8708 | 2:53799255 | 8721 | 2:54148820 | 8743 | 2:54689808 | 8751 | 2:54852723 | 8752 | 2:54874680 |
| 8786 | 2:55757496 | 8788 | 2:55856268 | 8836 | 2:57348471 | 8850 | 2:57670111 | 8867 | 2:58206752 |
| 8868 | 2:58209897 | 8870 | 2:58220436 | 8871 | 2:58224932 | 8872 | 2:58235017 | 8895 | 2:58894479 |
| 8937 | 2:59886642 | 8939 | 2:59894079 | 8941 | 2:59895422 | 8953 | 2:60262781 | 8986 | 2:61265032 |
| 9027 | 2:62337521 | 9064 | 2:63236556 | 9082 | 2:63698530 | 9103 | 2:64357206 | 9104 | 2:64367151 |
| 9106 | 2:64369812 | 9108 | 2:64437590 | 9109 | 2:64438167 | 9110 | 2:64438313 | 9111 | 2:64438576 |
| 9112 | 2:64438580 | 9113 | 2:64438635 | 9114 | 2:64438908 | 9122 | 2:64709919 | 9123 | 2:64711805 |
| 9144 | 2:65152676 | 9170 | 2:65933901 | 9210 | 2:67245827 | 9238 | 2:68102588 | 9241 | 2:68162816 |
| 9257 | 2:68641959 | 9282 | 2:69654612 | 9286 | 2:69797972 | 9289 | 2:69913137 | 9309 | 2:70434838 |
| 9326 | 2:70835144 | 9351 | 2:71366915 | 9354 | 2:71385196 | 9380 | 2:72368600 | 9431 | 2:73656519 |
| 9432 | 2:73692943 | 9438 | 2:73821323 | 9450 | 2:74104600 | 9460 | 2:74465077 | 9499 | 2:75629903 |
| 9522 | 2:76151639 | 9528 | 2:76318715 | 9572 | 2:77446117 | 9613 | 2:78673595 | 9630 | 2:78917434 |
| 9682 | 2:80074128 | 9733 | 2:82257633 | 9802 | 2:84430538 | 9875 | 2:86138420 | 9884 | 2:86394912 |
| 10120 | 2:91927539 | 10135 | 2:92191854 | 10175 | 2:93130350 | 10210 | 2:93703916 | 10252 | 2:94676418 |
| 10283 | 2:95426880 | 10287 | 2:95450849 | 10300 | 2:95820889 | 10323 | 2:96340062 | 10366 | 2:97323100 |
| 10378 | 2:97726887 | 10382 | 2:97746848 | 10396 | 2:98170549 | 10402 | 2:98356021 | 10418 | 2:98570804 |
| 10421 | 2:98681146 | 10424 | 2:98710984 | 10458 | 2:99816412 | 10462 | 2:99892472 | 10475 | 2:100046086 |
| 10476 | 2:100060098 | 10482 | 2:100180128 | 10493 | 2:100545847 | 10503 | 2:100804647 | 10519 | 2:101319592 |
| 10560 | 2:102476291 | 10585 | 2:103244299 | 10601 | 2:103704057 | 10604 | 2:103752303 | 10606 | 2:103798942 |
| 10619 | 2:104096932 | 10723 | 2:106585047 | 10741 | 2:106795862 | 10757 | 2:107198319 | 10759 | 2:107259324 |
| 10775 | 2:107639270 | 10795 | 2:107803660 | 10816 | 2:108400383 | 10844 | 2:108979070 | 10883 | 2:109843763 |
| 10889 | 2:109962178 | 10898 | 2:110266270 | 10908 | 2:110533827 | 10913 | 2:110782529 | 10921 | 2:111164890 |
| 10941 | 2:111820434 | 10980 | 2:112795307 | 11039 | 2:113925261 | 11047 | 2:114161924 | 11082 | 2:115099463 |
| 11086 | 2:115273767 | 11117 | 2:115778796 | 11118 | 2:115812040 | 11122 | 2:115883386 | 11129 | 2:116006798 |
| 11136 | 2:116182220 | 11144 | 2:116499676 | 11175 | 2:117182401 | 11219 | 2:118492898 | 11223 | 2:118587365 |
| 11228 | 2:118743982 | 11256 | 2:119406723 | 11265 | 2:119542337 | 11296 | 2:120281653 | 11318 | 2:120899441 |
| 11346 | 2:121587712 | 11400 | 2:123193388 | 11465 | 2:125068403 | 11477 | 2:125260143 | 11482 | 2:125405078 |
| 11524 | 2:126369897 | 11535 | 2:126645449 | 11566 | 2:126956287 | 11600 | 2:127060935 | 11611 | 2:127195966 |
| 11616 | 2:127259539 | 11643 | 2:127688853 | 11647 | 2:127843626 | 11660 | 2:128258137 | 11661 | 2:128269460 |
| 11739 | 2:129924952 | 11741 | 2:130000075 | 11795 | 2:131203320 | 11796 | 2:131203333 | 11797 | 2:131206099 |
| 11802 | 2:131272766 | 11848 | 2:132287345 | 11863 | 2:132630938 | 11864 | 2:132635825 | 11865 | 2:132637425 |
| 11922 | 2:133672767 | 11948 | 2:133927517 | 11962 | 2:134187671 | 11976 | 2:134370377 | 12003 | 2:134731903 |
| 12072 | 2:135676512 | 12100 | 3:74989 | 12107 | 3:258342 | 12138 | 3:907384 | 12182 | 3:1660018 |
| 12186 | 3:1722820 | 12267 | 3:3478015 | 12291 | 3:4097674 | 12293 | 3:4100027 | 12301 | 3:4206473 |
| 12342 | 3:4711535 | 12358 | 3:4936089 | 12364 | 3:5121045 | 12365 | 3:5121673 | 12366 | 3:5126160 |
| 12367 | 3:5130659 | 12386 | 3:5499346 | 12429 | 3:6474877 | 12436 | 3:6635006 | 12448 | 3:6773779 |
| 12474 | 3:7163463 | 12482 | 3:7308711 | 12520 | 3:7916080 | 12530 | 3:7997693 | 12589 | 3:9507918 |
| 12593 | 3:9562991 | 12600 | 3:9657041 | 12615 | 3:9990546 | 12621 | 3:10103527 | 12678 | 3:10496754 |
| 12726 | 3:11145282 | 12768 | 3:12417249 | 12776 | 3:12650225 | 12779 | 3:12749123 | 12804 | 3:13251625 |
| 12806 | 3:13370163 | 12807 | 3:13373604 | 12808 | 3:13375404 | 12812 | 3:13573971 | 12833 | 3:13902991 |
| 12866 | 3:14463788 | 12898 | 3:15197212 | 12926 | 3:15692187 | 12941 | 3:15905069 | 12972 | 3:16573676 |
| 12993 | 3:16936423 | 13136 | 3:21101166 | 13141 | 3:21359229 | 13158 | 3:21799171 | 13166 | 3:21978447 |
| 13170 | 3:22029801 | 13230 | 3:23274662 | 13295 | 3:24786196 | 13318 | 3:25281477 | 13329 | 3:25568097 |
| 13394 | 3:26947828 | 13404 | 3:27140090 | 13421 | 3:27417464 | 13426 | 3:27531689 | 13432 | 3:27586695 |
| 13433 | 3:27587568 | 13434 | 3:27588165 | 13435 | 3:27590766 | 13436 | 3:27590979 | 13437 | 3:27591026 |
| 13438 | 3:27591357 | 13439 | 3:27591992 | 13440 | 3:27592060 | 13441 | 3:27592156 | 13442 | 3:27592425 |
| 13443 | 3:27592963 | 13447 | 3:27618230 | 13448 | 3:27623256 | 13449 | 3:27624318 | 13450 | 3:27624378 |
| 13451 | 3:27626096 | 13488 | 3:28582812 | 13489 | 3:28655869 | 13493 | 3:28699418 | 13494 | 3:28701817 |
| 13496 | 3:28737754 | 13497 | 3:28739308 | 13502 | 3:28795698 | 13503 | 3:28847511 | 13512 | 3:29091766 |
| 13517 | 3:29129658 | 13528 | 3:29280162 | 13530 | 3:29285000 | 13543 | 3:29574496 | 13776 | 3:33446479 |
| 13782 | 3:33558867 | 13874 | 3:35055289 | 13935 | 3:36314410 | 13938 | 3:36323470 | 13942 | 3:36436943 |
| 13943 | 3:36535092 | 13955 | 3:36838325 | 13956 | 3:36846776 | 13971 | 3:37286638 | 14025 | 3:39287939 |
| 14031 | 3:39484220 | 14091 | 3:41318467 | 14096 | 3:41450661 | 14106 | 3:41608150 | 14116 | 3:41941631 |
| 14121 | 3:42042149 | 14131 | 3:42370072 | 14173 | 3:43226312 | 14205 | 3:43968451 | 14208 | 3:44136086 |
| 14252 | 3:45371773 | 14253 | 3:45389341 | 14259 | 3:45661559 | 14287 | 3:46609755 | 14319 | 3:47528437 |
| 14325 | 3:47614770 | 14335 | 3:47862102 | 14340 | 3:47901200 | 14346 | 3:48004343 | 14371 | 3:48662558 |
| 14376 | 3:48794169 | 14383 | 3:48908474 | 14388 | 3:48945044 | 14391 | 3:48996518 | 14393 | 3:49005907 |
| 14397 | 3:49066263 | 14400 | 3:49170559 | 14411 | 3:49437426 | 14430 | 3:49640985 | 14431 | 3:49686014 |
| 14432 | 3:49688113 | 14433 | 3:49693943 | 14434 | 3:49694205 | 14435 | 3:49694911 | 14436 | 3:49695537 |
| 14437 | 3:49695765 | 14438 | 3:49695863 | 14439 | 3:49695894 | 14440 | 3:49696034 | 14441 | 3:49697009 |
| 14442 | 3:49697975 | 14443 | 3:49698099 | 14444 | 3:49698127 | 14445 | 3:49698364 | 14446 | 3:49698389 |
| 14447 | 3:49698398 | 14448 | 3:49698433 | 14449 | 3:49698581 | 14450 | 3:49698599 | 14451 | 3:49698610 |
| 14452 | 3:49698697 | 14453 | 3:49698897 | 14454 | 3:49698954 | 14455 | 3:49698961 | 14456 | 3:49699145 |
| 14457 | 3:49699438 | 14458 | 3:49699942 | 14459 | 3:49700029 | 14460 | 3:49700320 | 14461 | 3:49700692 |
| 14462 | 3:49700695 | 14463 | 3:49700919 | 14464 | 3:49701129 | 14465 | 3:49701149 | 14466 | 3:49701225 |
| 14467 | 3:49701410 | 14468 | 3:49701470 | 14469 | 3:49702278 | 14470 | 3:49702301 | 14476 | 3:49768343 |
| 14489 | 3:50198427 | 14509 | 3:50849745 | 14526 | 3:51610373 | 14542 | 3:51914443 | 14546 | 3:51963753 |
| 14547 | 3:52005921 | 14550 | 3:52127559 | 14559 | 3:52354237 | 14560 | 3:52412518 | 14562 | 3:52416849 |
| 14563 | 3:52423182 | 14564 | 3:52427169 | 14566 | 3:52429848 | 14567 | 3:52433914 | 14570 | 3:52487075 |
| 14577 | 3:52690753 | 14580 | 3:52745287 | 14583 | 3:52852927 | 14591 | 3:53113743 | 14592 | 3:53117054 |
| 14593 | 3:53126092 | 14594 | 3:53126314 | 14595 | 3:53126332 | 14597 | 3:53143352 | 14600 | 3:53244194 |
| 14608 | 3:53474847 | 14610 | 3:53481481 | 14611 | 3:53510393 | 14652 | 3:55080090 | 14670 | 3:55424410 |
| 14740 | 3:57665967 | 14760 | 3:58213744 | 14761 | 3:58216986 | 14771 | 3:58501632 | 14789 | 3:58991070 |
| 14855 | 3:60699483 | 14856 | 3:60699729 | 14857 | 3:60704807 | 14871 | 3:60976891 | 14874 | 3:61002539 |
| 14876 | 3:61050627 | 14911 | 3:62289881 | 14936 | 3:62942816 | 14979 | 3:64119969 | 14994 | 3:64762588 |
| 15003 | 3:65001585 | 15011 | 3:65190175 | 15045 | 3:66062653 | 15056 | 3:66307909 | 15064 | 3:66462222 |
| 15108 | 3:67467438 | 15119 | 3:67681424 | 15160 | 3:68627230 | 15190 | 3:69504184 | 15196 | 3:69782248 |
| 15197 | 3:69782256 | 15198 | 3:69782262 | 15199 | 3:69786350 | 15215 | 3:70251128 | 15260 | 3:71848961 |
| 15262 | 3:71934380 | 15307 | 3:73666374 | 15325 | 3:74232466 | 15331 | 3:74366602 | 15394 | 3:76166365 |
| 15408 | 3:76630621 | 15458 | 3:78060366 | 15471 | 3:78544662 | 15539 | 3:80535834 | 15585 | 3:81521406 |
| 15586 | 3:81525295 | 15639 | 3:82930218 | 15642 | 3:83051313 | 15674 | 3:84083709 | 15679 | 3:84152801 |
| 15685 | 3:84286878 | 15713 | 3:84688663 | 15738 | 3:84955518 | 15745 | 3:85050052 | 15746 | 3:85066223 |
| 15766 | 3:85749188 | 15769 | 3:85769939 | 15774 | 3:85846088 | 15776 | 3:85897724 | 15784 | 3:85979828 |
| 15785 | 3:86001057 | 15786 | 3:86027116 | 15846 | 3:87314340 | 15854 | 3:87568457 | 15855 | 3:87570687 |
| 15857 | 3:87576354 | 15858 | 3:87576887 | 15859 | 3:87596560 | 15865 | 3:87888225 | 15900 | 3:88530363 |
| 15903 | 3:88603888 | 15905 | 3:88620871 | 15926 | 3:88972091 | 15934 | 3:89181812 | 15938 | 3:89279883 |
| 15955 | 3:89599405 | 15989 | 3:90306732 | 15990 | 3:90333561 | 16033 | 3:91219484 | 16036 | 3:91278796 |
| 16051 | 3:91695764 | 16084 | 3:92543467 | 16101 | 3:92905285 | 16113 | 3:93070586 | 16148 | 3:93889901 |
| 16213 | 3:95744017 | 16218 | 3:95893190 | 16222 | 3:96032684 | 16228 | 3:96151926 | 16281 | 3:97566938 |
| 16299 | 3:97901912 | 16346 | 3:99237470 | 16360 | 3:99532212 | 16362 | 3:99579446 | 16424 | 3:100686242 |
| 16429 | 3:100725968 | 16463 | 3:101430182 | 16476 | 3:101718735 | 16488 | 3:102039758 | 16507 | 3:102478118 |
| 16508 | 3:102487038 | 16525 | 3:102814655 | 16545 | 3:103051617 | 16547 | 3:103073339 | 16685 | 3:105603357 |
| 16698 | 3:105784125 | 16760 | 3:106768484 | 16763 | 3:106821154 | 16796 | 3:107517305 | 16797 | 3:107520457 |
| 16805 | 3:107754888 | 16842 | 3:108517017 | 16865 | 3:108844131 | 16904 | 3:109724431 | 16905 | 3:109725745 |
| 16906 | 3:109730150 | 16907 | 3:109731953 | 16908 | 3:109737608 | 16909 | 3:109737977 | 16910 | 3:109738917 |
| 16980 | 3:111463594 | 16984 | 3:111515807 | 17006 | 3:111954834 | 17039 | 3:112612031 | 17040 | 3:112625613 |
| 17055 | 3:112990876 | 17088 | 3:113561927 | 17123 | 3:114126601 | 17131 | 3:114256669 | 17145 | 3:114503215 |
| 17147 | 3:114519254 | 17151 | 3:114571821 | 17153 | 3:114600016 | 17154 | 3:114600017 | 17161 | 3:114722650 |
| 17203 | 3:115249589 | 17205 | 3:115271507 | 17250 | 3:116030538 | 17298 | 3:116861627 | 17300 | 3:116869634 |
| 17301 | 3:116871170 | 17303 | 3:116878333 | 17305 | 3:116901187 | 17306 | 3:116901527 | 17307 | 3:116903799 |
| 17311 | 3:116932009 | 17340 | 3:117275171 | 17356 | 3:117642247 | 17357 | 3:117642545 | 17358 | 3:117643811 |
| 17359 | 3:117644307 | 17398 | 3:118174177 | 17427 | 3:118632548 | 17445 | 3:118859735 | 17446 | 3:118864123 |
| 17451 | 3:118890670 | 17500 | 3:120280786 | 17594 | 4:2379436 | 17634 | 4:3851682 | 17635 | 4:3859753 |
| 17638 | 4:3982830 | 17645 | 4:4155060 | 17665 | 4:4566566 | 17667 | 4:4584480 | 17668 | 4:4589135 |
| 17669 | 4:4591392 | 17670 | 4:4591448 | 17677 | 4:4684194 | 17681 | 4:4780212 | 17682 | 4:4780800 |
| 17683 | 4:4786841 | 17687 | 4:4824857 | 17690 | 4:4865949 | 17694 | 4:4969955 | 17719 | 4:5368040 |
| 17727 | 4:5444338 | 17738 | 4:5704758 | 17765 | 4:6383685 | 17791 | 4:7064916 | 17803 | 4:7319609 |
| 17805 | 4:7325326 | 17815 | 4:7454936 | 17830 | 4:7796044 | 17846 | 4:8193002 | 17869 | 4:8790081 |
| 17881 | 4:9092344 | 17895 | 4:9372959 | 17903 | 4:9479800 | 17917 | 4:9747477 | 17933 | 4:9983918 |
| 17934 | 4:9983950 | 17935 | 4:9984121 | 17936 | 4:9984185 | 17937 | 4:9984248 | 17938 | 4:9984598 |
| 17940 | 4:9986515 | 17971 | 4:10645340 | 17974 | 4:10692378 | 17979 | 4:10807167 | 17980 | 4:10817042 |
| 17982 | 4:10817118 | 18028 | 4:11882269 | 18081 | 4:13277909 | 18083 | 4:13314057 | 18084 | 4:13315343 |
| 18132 | 4:14870332 | 18185 | 4:16314407 | 18193 | 4:16510166 | 18212 | 4:17151245 | 18220 | 4:17227279 |
| 18227 | 4:17429209 | 18244 | 4:17664281 | 18251 | 4:17934274 | 18292 | 4:18879751 | 18295 | 4:18927680 |
| 18301 | 4:18998283 | 18302 | 4:18999825 | 18303 | 4:19004726 | 18304 | 4:19005718 | 18305 | 4:19006333 |
| 18306 | 4:19007854 | 18307 | 4:19007876 | 18308 | 4:19008526 | 18309 | 4:19008760 | 18310 | 4:19010468 |
| 18331 | 4:19684498 | 18332 | 4:19687001 | 18333 | 4:19687703 | 18334 | 4:19688039 | 18335 | 4:19690469 |
| 18336 | 4:19690711 | 18337 | 4:19692144 | 18338 | 4:19692593 | 18339 | 4:19692676 | 18340 | 4:19693098 |
| 18341 | 4:19693119 | 18342 | 4:19694330 | 18343 | 4:19694937 | 18344 | 4:19695576 | 18345 | 4:19695681 |
| 18346 | 4:19696720 | 18347 | 4:19696767 | 18348 | 4:19697089 | 18349 | 4:19697301 | 18350 | 4:19697430 |
| 18351 | 4:19697544 | 18352 | 4:19697663 | 18354 | 4:19748494 | 18359 | 4:19938692 | 18361 | 4:19989444 |
| 18375 | 4:20261489 | 18376 | 4:20265073 | 18392 | 4:20703476 | 18401 | 4:20822864 | 18421 | 4:21167294 |
| 18431 | 4:21342092 | 18432 | 4:21358199 | 18440 | 4:21699344 | 18457 | 4:22138635 | 18466 | 4:22471078 |
| 18468 | 4:22523960 | 18481 | 4:22728193 | 18490 | 4:22923982 | 18497 | 4:23121119 | 18500 | 4:23196768 |
| 18504 | 4:23389777 | 18530 | 4:24084837 | 18546 | 4:24614792 | 18563 | 4:25092475 | 18600 | 4:26062712 |
| 18625 | 4:26743642 | 18628 | 4:26796377 | 18632 | 4:26851816 | 18643 | 4:27158677 | 18656 | 4:27519461 |
| 18679 | 4:28114813 | 18697 | 4:28590705 | 18717 | 4:29227466 | 18733 | 4:29791373 | 18759 | 4:30557284 |
| 18768 | 4:30768290 | 18782 | 4:31109031 | 18806 | 4:31790626 | 18808 | 4:31802602 | 18827 | 4:32352959 |
| 18838 | 4:32687897 | 18870 | 4:33603343 | 18902 | 4:34379775 | 18952 | 4:36053918 | 18953 | 4:36057890 |
| 18966 | 4:36473548 | 18993 | 4:37223623 | 19001 | 4:37381316 | 19019 | 4:38054717 | 19024 | 4:38091098 |
| 19030 | 4:38294871 | 19041 | 4:38579078 | 19042 | 4:38591331 | 19060 | 4:39231026 | 19066 | 4:39400801 |
| 19084 | 4:39970721 | 19114 | 4:40595576 | 19133 | 4:40929985 | 19178 | 4:42197758 | 19207 | 4:43094045 |
| 19209 | 4:43138220 | 19236 | 4:43793806 | 19294 | 4:45527845 | 19336 | 4:46745755 | 19366 | 4:47300099 |
| 19380 | 4:47488474 | 19388 | 4:47696820 | 19425 | 4:49010238 | 19454 | 4:49228470 | 19465 | 4:49442617 |
| 19505 | 4:50866615 | 19529 | 4:51642850 | 19540 | 4:52014153 | 19574 | 4:53063969 | 19623 | 4:54533598 |
| 19654 | 4:55652059 | 19655 | 4:55666248 | 19666 | 4:55979945 | 19678 | 4:56152616 | 19746 | 4:58369087 |
| 19753 | 4:58591231 | 19754 | 4:58596005 | 19769 | 4:59119437 | 19775 | 4:59214261 | 19791 | 4:59626845 |
| 19794 | 4:59754417 | 19815 | 4:60395032 | 19869 | 4:61855772 | 19915 | 4:63120245 | 19920 | 4:63231993 |
| 19939 | 4:63706634 | 19941 | 4:63737742 | 19965 | 4:64527362 | 19979 | 4:64927929 | 19981 | 4:64973879 |
| 20073 | 4:67329757 | 20080 | 4:67492078 | 20083 | 4:67548081 | 20121 | 4:68635250 | 20122 | 4:68672213 |
| 20195 | 4:70609606 | 20233 | 4:71767720 | 20235 | 4:71789741 | 20358 | 4:75159207 | 20362 | 4:75242210 |
| 20385 | 4:75797380 | 20413 | 4:76318250 | 20436 | 4:77029452 | 20481 | 4:77948146 | 20491 | 4:78185575 |
| 20539 | 4:79657303 | 20543 | 4:79839208 | 20568 | 4:80186925 | 20573 | 4:80245076 | 20587 | 4:80584868 |
| 20592 | 4:80719912 | 20661 | 4:82586451 | 20666 | 4:82698761 | 20712 | 4:83936797 | 20729 | 4:84451586 |
| 20765 | 4:85525303 | 20774 | 4:85682095 | 20781 | 4:85880701 | 20796 | 4:86159050 | 20846 | 4:87474051 |
| 20850 | 4:87586277 | 20873 | 4:88094522 | 20894 | 4:88685340 | 20914 | 4:89467898 | 20924 | 4:89712815 |
| 20974 | 4:90944945 | 21037 | 4:92282682 | 21054 | 4:92473823 | 21057 | 4:92523126 | 21063 | 4:92640436 |
| 21103 | 4:93420321 | 21105 | 4:93454704 | 21113 | 4:93574942 | 21118 | 4:93658997 | 21176 | 4:95336071 |
| 21177 | 4:95348378 | 21181 | 4:95493235 | 21227 | 4:96019036 | 21262 | 4:96809210 | 21279 | 4:96825379 |
| 21360 | 4:96942694 | 21366 | 4:97025110 | 21436 | 4:97663817 | 21456 | 4:98220002 | 21526 | 4:100120727 |
| 21537 | 4:100380798 | 21545 | 4:100648109 | 21550 | 4:100737518 | 21555 | 4:100801331 | 21644 | 4:102776227 |
| 21654 | 4:103076079 | 21677 | 4:103575595 | 21680 | 4:103670470 | 21746 | 4:105364910 | 21757 | 4:105516749 |
| 21761 | 4:105568960 | 21777 | 4:106062418 | 21819 | 4:106715919 | 21888 | 4:107648399 | 21906 | 4:108309499 |
| 21907 | 4:108319723 | 21926 | 4:108947713 | 21940 | 4:109363070 | 21947 | 4:109525615 | 21948 | 4:109528067 |
| 21949 | 4:109528137 | 21950 | 4:109529095 | 21951 | 4:109529531 | 21952 | 4:109529976 | 21953 | 4:109530257 |
| 21954 | 4:109530338 | 21955 | 4:109532512 | 21956 | 4:109533036 | 21957 | 4:109538711 | 21958 | 4:109538973 |
| 21959 | 4:109545654 | 21961 | 4:109576187 | 21985 | 4:110235765 | 21986 | 4:110257114 | 21999 | 4:110637707 |
| 22000 | 4:110672104 | 22001 | 4:110703849 | 22014 | 4:110890327 | 22025 | 4:111120368 | 22026 | 4:111120557 |
| 22061 | 4:111707261 | 22062 | 4:111708263 | 22063 | 4:111711546 | 22064 | 4:111711848 | 22065 | 4:111713444 |
| 22086 | 4:112129918 | 22089 | 4:112149705 | 22095 | 4:112210325 | 22107 | 4:112369141 | 22119 | 4:112555531 |
| 22179 | 4:113489475 | 22192 | 4:113598124 | 22193 | 4:113600602 | 22194 | 4:113601769 | 22196 | 4:113610307 |
| 22197 | 4:113612170 | 22198 | 4:113614202 | 22202 | 4:113636177 | 22203 | 4:113637533 | 22204 | 4:113639510 |
| 22205 | 4:113645913 | 22206 | 4:113646772 | 22207 | 4:113647228 | 22208 | 4:113647637 | 22209 | 4:113649086 |
| 22210 | 4:113650751 | 22211 | 4:113653050 | 22212 | 4:113670640 | 22213 | 4:113670882 | 22214 | 4:113671866 |
| 22215 | 4:113673914 | 22216 | 4:113673955 | 22217 | 4:113676621 | 22218 | 4:113680315 | 22351 | 4:115923041 |
| 22368 | 4:116278293 | 22377 | 4:116438965 | 22393 | 4:116746277 | 22401 | 4:116897937 | 22404 | 4:116926240 |
| 22405 | 4:116961074 | 22514 | 4:119167733 | 22520 | 4:119293731 | 22547 | 5:35727 | 22548 | 5:42965 |
| 22549 | 5:46543 | 22550 | 5:56066 | 22551 | 5:57672 | 22554 | 5:90000 | 22555 | 5:93744 |
| 22556 | 5:95203 | 22557 | 5:102783 | 22558 | 5:126748 | 22559 | 5:149452 | 22560 | 5:156011 |
| 22562 | 5:157715 | 22563 | 5:159858 | 22578 | 5:818560 | 22609 | 5:1354890 | 22616 | 5:1388630 |
| 22619 | 5:1458836 | 22642 | 5:1946239 | 22703 | 5:3492443 | 22727 | 5:4322265 | 22743 | 5:4570191 |
| 22752 | 5:4731333 | 22754 | 5:4732613 | 22775 | 5:5220278 | 22799 | 5:5717840 | 22851 | 5:6927428 |
| 22853 | 5:6949366 | 22892 | 5:7781205 | 22896 | 5:7895175 | 22903 | 5:8176883 | 22949 | 5:9380271 |
| 22955 | 5:9551052 | 22956 | 5:9555919 | 22960 | 5:9636042 | 22996 | 5:10313930 | 23013 | 5:10668196 |
| 23026 | 5:10924954 | 23029 | 5:10994036 | 23065 | 5:12039811 | 23100 | 5:12501985 | 23102 | 5:12524034 |
| 23125 | 5:13140668 | 23167 | 5:14461980 | 23181 | 5:14793206 | 23214 | 5:15447067 | 23218 | 5:15558924 |
| 23257 | 5:16336560 | 23268 | 5:16719670 | 23269 | 5:16739498 | 23279 | 5:17077362 | 23280 | 5:17078096 |
| 23292 | 5:17322229 | 23296 | 5:17492634 | 23298 | 5:17500602 | 23301 | 5:17665160 | 23307 | 5:17903579 |
| 23313 | 5:18155232 | 23326 | 5:18518099 | 23364 | 5:19695581 | 23400 | 5:20738200 | 23430 | 5:21391973 |
| 23432 | 5:21432976 | 23439 | 5:21643782 | 23440 | 5:21648427 | 23451 | 5:21871823 | 23459 | 5:21991889 |
| 23484 | 5:22479526 | 23485 | 5:22487130 | 23543 | 5:23622599 | 23555 | 5:23928759 | 23558 | 5:23956744 |
| 23613 | 5:25183734 | 23665 | 5:26532385 | 23677 | 5:26846005 | 23680 | 5:26972277 | 23681 | 5:26972772 |
| 23690 | 5:27127258 | 23702 | 5:27439061 | 23707 | 5:27544684 | 23733 | 5:27949133 | 23757 | 5:28498202 |
| 23799 | 5:29557818 | 23812 | 5:29809074 | 23822 | 5:29994317 | 23825 | 5:30019258 | 23946 | 5:33177185 |
| 23952 | 5:33331535 | 23953 | 5:33336405 | 23954 | 5:33337110 | 23968 | 5:33738237 | 24007 | 5:34880473 |
| 24042 | 5:35923865 | 24045 | 5:36012922 | 24048 | 5:36038265 | 24049 | 5:36042606 | 24058 | 5:36409810 |
| 24074 | 5:36939207 | 24120 | 5:37996697 | 24182 | 5:39657770 | 24190 | 5:39821210 | 24193 | 5:39927222 |
| 24238 | 5:41211155 | 24240 | 5:41232876 | 24241 | 5:41235105 | 24242 | 5:41236186 | 24258 | 5:41776747 |
| 24279 | 5:42522952 | 24322 | 5:43563557 | 24327 | 5:43634786 | 24366 | 5:44572552 | 24385 | 5:45181510 |
| 24386 | 5:45181566 | 24402 | 5:45615706 | 24417 | 5:45973787 | 24431 | 5:46166472 | 24441 | 5:46464570 |
| 24443 | 5:46517477 | 24452 | 5:46782312 | 24458 | 5:46936034 | 24459 | 5:46938241 | 24460 | 5:46938449 |
| 24461 | 5:46940754 | 24462 | 5:46941677 | 24475 | 5:47204347 | 24522 | 5:48389782 | 24529 | 5:48667259 |
| 24535 | 5:48760782 | 24540 | 5:48866791 | 24550 | 5:49202198 | 24559 | 5:49562957 | 24562 | 5:49625601 |
| 24570 | 5:49842789 | 24607 | 5:50836826 | 24779 | 5:55323383 | 24806 | 5:55945914 | 24858 | 5:57094271 |
| 24893 | 5:58015125 | 24920 | 5:59115316 | 24926 | 5:59337375 | 24942 | 5:59884753 | 24948 | 5:60008612 |
| 24991 | 5:60879895 | 24994 | 5:60984488 | 24999 | 5:61164840 | 25018 | 5:61884469 | 25034 | 5:62444037 |
| 25035 | 5:62445409 | 25036 | 5:62449226 | 25037 | 5:62449848 | 25038 | 5:62450932 | 25055 | 5:63033268 |
| 25082 | 5:63536637 | 25111 | 5:64402956 | 25124 | 5:64829651 | 25128 | 5:64926399 | 25129 | 5:64926523 |
| 25130 | 5:64928264 | 25164 | 5:65659261 | 25171 | 5:65755099 | 25197 | 5:66595784 | 25208 | 5:66799811 |
| 25219 | 5:67271370 | 25335 | 5:69618545 | 25426 | 5:71726794 | 25464 | 5:72378602 | 25466 | 5:72402551 |
| 25482 | 5:73010927 | 25486 | 5:73094311 | 25488 | 5:73189660 | 25550 | 5:74746217 | 25597 | 5:75667991 |
| 25606 | 5:75883986 | 25611 | 5:75946414 | 25665 | 5:77347106 | 25680 | 5:77945857 | 25725 | 5:78973946 |
| 25727 | 5:79017627 | 25735 | 5:79334960 | 25748 | 5:79714596 | 25749 | 5:79722250 | 25781 | 5:80877376 |
| 25791 | 5:81278052 | 25871 | 5:82792399 | 25884 | 5:83174887 | 25891 | 5:83348374 | 25900 | 5:83570417 |
| 25915 | 5:83863121 | 25918 | 5:83916047 | 25923 | 5:84010266 | 25926 | 5:84064602 | 25927 | 5:84064623 |
| 25928 | 5:84064772 | 25929 | 5:84065038 | 25930 | 5:84065171 | 25931 | 5:84065332 | 25932 | 5:84065339 |
| 25933 | 5:84065355 | 25934 | 5:84066088 | 25935 | 5:84066287 | 25936 | 5:84067645 | 25937 | 5:84068297 |
| 25938 | 5:84068606 | 25939 | 5:84068687 | 25940 | 5:84068732 | 2594 | 5:84068756 | 25942 | 5:84070079 |
| 25943 | 5:84070128 | 25944 | 5:84076319 | 25945 | 5:84076527 | 25946 | 5:84077100 | 25947 | 5:84080168 |
| 25949 | 5:84086306 | 25950 | 5:84086861 | 25951 | 5:84090372 | 25952 | 5:84090487 | 25953 | 5:84097284 |
| 25954 | 5:84097885 | 25955 | 5:84097985 | 25956 | 5:84099214 | 25957 | 5:84100319 | 25958 | 5:84101337 |
| 25959 | 5:84101438 | 25960 | 5:84101462 | 25961 | 5:84101494 | 25962 | 5:84101519 | 25963 | 5:84101555 |
| 25964 | 5:84101619 | 25965 | 5:84101701 | 25966 | 5:84102206 | 25967 | 5:84102236 | 25969 | 5:84103792 |
| 25970 | 5:84103930 | 25971 | 5:84104200 | 25972 | 5:84107013 | 25974 | 5:84115513 | 25975 | 5:84115550 |
| 25976 | 5:84115605 | 25977 | 5:84115682 | 25978 | 5:84115698 | 25979 | 5:84115763 | 25980 | 5:84115908 |
| 25981 | 5:84116028 | 25982 | 5:84116506 | 25983 | 5:84116583 | 25984 | 5:84119378 | 25985 | 5:84119408 |
| 25986 | 5:84119435 | 25987 | 5:84120471 | 25988 | 5:84120477 | 25989 | 5:84120540 | 25990 | 5:84120562 |
| 25991 | 5:84120612 | 25992 | 5:84120761 | 25993 | 5:84120772 | 25994 | 5:84120800 | 25995 | 5:84120828 |
| 25996 | 5:84120900 | 25997 | 5:84120980 | 25998 | 5:84120987 | 25999 | 5:84121087 | 26000 | 5:84121318 |
| 26001 | 5:84122651 | 26002 | 5:84122709 | 26003 | 5:84122905 | 26004 | 5:84123385 | 26005 | 5:84123428 |
| 26006 | 5:84124508 | 26007 | 5:84124539 | 26008 | 5:84124557 | 26009 | 5:84124558 | 26010 | 5:84124682 |
| 26011 | 5:84125648 | 26012 | 5:84125855 | 26013 | 5:84126181 | 26014 | 5:84126183 | 26015 | 5:84126222 |
| 26016 | 5:84126766 | 26017 | 5:84126941 | 26018 | 5:84126965 | 26019 | 5:84126986 | 26020 | 5:84127998 |
| 26021 | 5:84128048 | 26022 | 5:84128850 | 26023 | 5:84128968 | 26024 | 5:84129307 | 26025 | 5:84129349 |
| 26026 | 5:84129709 | 26027 | 5:84130342 | 26028 | 5:84130419 | 26029 | 5:84130836 | 26030 | 5:84130988 |
| 26031 | 5:84131404 | 26032 | 5:84131699 | 26033 | 5:84131703 | 26034 | 5:84131742 | 26035 | 5:84132713 |
| 26036 | 5:84132801 | 26037 | 5:84132954 | 26038 | 5:84133129 | 26039 | 5:84133144 | 26040 | 5:84133170 |
| 26041 | 5:84133302 | 26042 | 5:84133384 | 26043 | 5:84133400 | 26044 | 5:84133418 | 26045 | 5:84135047 |
| 26046 | 5:84135179 | 26047 | 5:84135274 | 26048 | 5:84137923 | 26049 | 5:84138207 | 26050 | 5:84138277 |
| 26051 | 5:84138403 | 26052 | 5:84138617 | 26053 | 5:8413864 | 26054 | 5:84138927 | 26055 | 5:84142947 |
| 26056 | 5:84143312 | 26069 | 5:84405330 | 26105 | 5:85329574 | 26123 | 5:85783695 | 26129 | 5:85892084 |
| 26147 | 5:86451423 | 26153 | 5:86534299 | 26158 | 5:86590201 | 26161 | 5:86629956 | 26175 | 5:86950590 |
| 26207 | 5:87624956 | 26209 | 5:87645403 | 26219 | 5:87859562 | 26287 | 5:88745049 | 26319 | 5:89399078 |
| 26320 | 5:89427836 | 26334 | 5:89821006 | 26385 | 5:91059021 | 26400 | 5:91433996 | 26401 | 5:91479219 |
| 26434 | 5:92344711 | 26436 | 5:92351975 | 26437 | 5:92360410 | 26440 | 5:92389962 | 26441 | 5:92399584 |
| 26442 | 5:92402496 | 26443 | 5:92402509 | 26444 | 5:92407767 | 26445 | 5:92412423 | 26446 | 5:92412426 |
| 26447 | 5:92412918 | 26448 | 5:92415826 | 26449 | 5:92416355 | 26450 | 5:92418513 | 26451 | 5:92422512 |
| 26452 | 5:92422802 | 26453 | 5:92423893 | 26454 | 5:92426805 | 26455 | 5:92428903 | 26456 | 5:92428912 |
| 26457 | 5:92429850 | 26458 | 5:92429875 | 26459 | 5:92430956 | 26460 | 5:92431470 | 26461 | 5:92431717 |
| 26462 | 5:92431719 | 26463 | 5:92432015 | 26464 | 5:92432232 | 26465 | 5:92432425 | 26466 | 5:92432633 |
| 26472 | 5:92541180 | 26487 | 5:92897747 | 26582 | 5:95173664 | 26584 | 5:95197902 | 26656 | 5:96676782 |
| 26698 | 5:97374091 | 26709 | 5:97622463 | 26728 | 5:98063850 | 26729 | 5:98064276 | 26735 | 5:98193113 |
| 26736 | 5:98202197 | 26738 | 5:98236533 | 26768 | 5:98748721 | 26772 | 5:98826571 | 26828 | 5:99948490 |
| 26861 | 5:100535488 | 26863 | 5:100631069 | 26870 | 5:100703214 | 26884 | 5:100953490 | 26893 | 5:101083268 |
| 26894 | 5:101089292 | 26899 | 5:101221558 | 26914 | 5:101607756 | 26921 | 5:101673726 | 26928 | 5:101914630 |
| 26940 | 5:102460767 | 26943 | 5:102476964 | 26968 | 5:103154349 | 26986 | 5:103552417 | 27001 | 5:103882706 |
| 27005 | 5:103981305 | 27068 | 5:105100005 | 27114 | 5:105867389 | 27140 | 5:106221085 | 27148 | 5:106293076 |
| 27162 | 5:106480592 | 27188 | 5:106887255 | 27202 | 5:107008747 | 27222 | 5:107342588 | 27232 | 5:107495491 |
| 27234 | 5:107522927 | 27269 | 5:108224683 | 27274 | 5:108258258 | 27280 | 5:108472906 | 27281 | 5:108473260 |
| 27282 | 5:108476147 | 27283 | 5:108478132 | 27284 | 5:108485933 | 27285 | 5:108486228 | 27286 | 5:108486756 |
| 27287 | 5:108487832 | 27288 | 5:108489677 | 27289 | 5:108490693 | 27290 | 5:108490734 | 27295 | 5:108546887 |
| 27297 | 5:108575310 | 27399 | 5:110511118 | 27440 | 5:111144555 | 27462 | 5:111761777 | 27467 | 5:111860816 |
| 27481 | 5:112115946 | 27519 | 5:112982896 | 27525 | 5:113133130 | 27539 | 5:113320727 | 27547 | 5:113650431 |
| 27566 | 5:114143008 | 27569 | 5:114206160 | 27572 | 5:114283857 | 27580 | 5:114719162 | 27583 | 5:114775926 |
| 27605 | 5:115427295 | 27652 | 5:116126971 | 27656 | 5:116179833 | 27676 | 5:116586289 | 27677 | 5:116588965 |
| 27678 | 5:116590488 | 27679 | 5:116590506 | 27680 | 5:116590745 | 27682 | 5:116592015 | 27683 | 5:116592619 |
| 27684 | 5:116592903 | 27687 | 5:116593192 | 27688 | 5:116593569 | 27694 | 5:116594398 | 27695 | 5:116594541 |
| 27696 | 5:116594546 | 27697 | 5:116594548 | 27698 | 5:116594847 | 27699 | 5:116595244 | 27700 | 5:116595727 |
| 27701 | 5:116595799 | 27702 | 5:116596186 | 27704 | 5:116596441 | 27705 | 5:116596625 | 27706 | 5:116596745 |
| 27707 | 5:116596913 | 27708 | 5:116597118 | 27711 | 5:116598702 | 27712 | 5:116598835 | 27715 | 5:116602527 |
| 27721 | 5:116677042 | 27731 | 5:116722878 | 27808 | 5:117709009 | 27809 | 5:117711361 | 27813 | 5:117773986 |
| 27826 | 5:117998759 | 27831 | 5:118098871 | 27838 | 5:118250217 | 27860 | 5:118703768 | 27866 | 5:118745750 |
| 27903 | 5:119271970 | 27908 | 5:119361103 | 27916 | 5:119587043 | 27948 | 6:528320 | 27970 | 6:993117 |
| 27999 | 6:1950764 | 28000 | 6:1951302 | 28001 | 6:1951498 | 28002 | 6:1951714 | 28003 | 6:1951719 |
| 28004 | 6:1951800 | 28005 | 6:1951981 | 28006 | 6:1952392 | 28007 | 6:1955233 | 28008 | 6:1955330 |
| 28009 | 6:1955401 | 28010 | 6:1955549 | 28011 | 6:1955698 | 28012 | 6:1956232 | 28013 | 6:1956278 |
| 28014 | 6:1957428 | 28015 | 6:1957620 | 28016 | 6:1957800 | 28017 | 6:1957812 | 28018 | 6:1957895 |
| 28019 | 6:1958152 | 28020 | 6:1962365 | 28021 | 6:1963113 | 28022 | 6:1963759 | 28023 | 6:1963825 |
| 28024 | 6:1963953 | 28026 | 6:1973036 | 28027 | 6:1974486 | 28028 | 6:1974895 | 28061 | 6:2801468 |
| 28063 | 6:2860306 | 28064 | 6:2862765 | 28072 | 6:3067202 | 28074 | 6:3073689 | 28075 | 6:3084367 |
| 28129 | 6:3786375 | 28155 | 6:4418616 | 28168 | 6:4782253 | 28170 | 6:4824255 | 28221 | 6:6133626 |
| 28238 | 6:6309983 | 28278 | 6:7061434 | 28286 | 6:7250536 | 28293 | 6:7394514 | 28319 | 6:8317563 |
| 28336 | 6:8929534 | 28337 | 6:8944681 | 28339 | 6:8955494 | 28341 | 6:8960665 | 28343 | 6:8972751 |
| 28345 | 6:8980990 | 28346 | 6:8985187 | 28347 | 6:8989449 | 28352 | 6:9079158 | 28353 | 6:9081859 |
| 28354 | 6:9085203 | 28356 | 6:9094449 | 28373 | 6:9291690 | 28377 | 6:9465992 | 28389 | 6:9964731 |
| 28390 | 6:9965161 | 28391 | 6:9976901 | 28393 | 6:9992803 | 28395 | 6:10029462 | 28409 | 6:10453731 |
| 28422 | 6:10849905 | 28435 | 6:11149738 | 28440 | 6:11315175 | 28605 | 6:15051875 | 28641 | 6:15680456 |
| 28686 | 6:16603866 | 28718 | 6:17241712 | 28757 | 6:18035615 | 28758 | 6:18035662 | 28759 | 6:18035884 |
| 28760 | 6:18036845 | 28766 | 6:18057767 | 28795 | 6:18806853 | 28819 | 6:19264271 | 28820 | 6:19264527 |
| 28852 | 6:19965110 | 28878 | 6:20517010 | 28888 | 6:20729903 | 28944 | 6:22097118 | 28970 | 6:22554116 |
| 29044 | 6:24253816 | 29057 | 6:24617112 | 29061 | 6:24750491 | 29069 | 6:24940966 | 29114 | 6:25994106 |
| 29115 | 6:25995000 | 29123 | 6:26081649 | 29151 | 6:26895539 | 29159 | 6:27119474 | 29179 | 6:27632577 |
| 29182 | 6:27765059 | 29183 | 6:27794604 | 29196 | 6:28156905 | 29204 | 6:28392137 | 29233 | 6:29155222 |
| 29374 | 6:30901671 | 29490 | 6:34238062 | 29722 | 6:34572571 | 29939 | 6:35180069 | 29996 | 6:35924006 |
| 29997 | 6:35924204 | 29998 | 6:35925489 | 29999 | 6:35931470 | 30000 | 6:35932579 | 30010 | 6:35978601 |
| 30133 | 6:36426239 | 30136 | 6:36429755 | 30138 | 6:36430937 | 30160 | 6:36467397 | 30164 | 6:36475104 |
| 30165 | 6:36477167 | 30168 | 6:36481433 | 30169 | 6:36481434 | 30171 | 6:36483188 | 30174 | 6:36489959 |
| 30175 | 6:36490754 | 30234 | 6:36545679 | 30259 | 6:36568156 | 30261 | 6:36568846 | 30328 | 6:36807893 |
| 30329 | 6:36808225 | 30459 | 6:37029628 | 30462 | 6:37032995 | 30468 | 6:37035541 | 30566 | 6:37095299 |
| 30653 | 6:37151905 | 30655 | 6:37152373 | 30679 | 6:37160005 | 30735 | 6:37172974 | 30736 | 6:37173048 |
| 30748 | 6:37175662 | 30750 | 6:37175773 | 30757 | 6:37176677 | 30772 | 6:37181087 | 30781 | 6:37182773 |
| 30787 | 6:37183901 | 30792 | 6:37189736 | 30794 | 6:37192102 | 30802 | 6:37214389 | 30852 | 6:37251132 |
| 30858 | 6:37262825 | 30921 | 6:37299855 | 31027 | 6:37329948 | 31093 | 6:37365265 | 31180 | 6:37431101 |
| 31284 | 6:37636883 | 31285 | 6:37636900 | 31524 | 6:37936087 | 31846 | 6:38545113 | 31894 | 6:38558946 |
| 31930 | 6:38800398 | 3193 | 6:38830763 | 31935 | 6:38855286 | 31936 | 6:38862311 | 31937 | 6:38862987 |
| 31938 | 6:38875748 | 31940 | 6:38879318 | 31941 | 6:38882135 | 31980 | 6:39565429 | 31983 | 6:39593110 |
| 32001 | 6:39758696 | 32010 | 6:39853149 | 32018 | 6:40036007 | 32020 | 6:40036943 | 32022 | 6:40040343 |
| 32023 | 6:40053452 | 32025 | 6:40058671 | 32030 | 6:40130949 | 32056 | 6:40611279 | 32063 | 6:40707783 |
| 32064 | 6:40708542 | 32065 | 6:40708988 | 32066 | 6:40709098 | 32067 | 6:40709130 | 32068 | 6:40709457 |
| 32069 | 6:40709727 | 32070 | 6:40709846 | 32071 | 6:40711310 | 32072 | 6:40711346 | 32073 | 6:40711409 |
| 32074 | 6:40711422 | 32075 | 6:40711439 | 32076 | 6:40713027 | 32077 | 6:40713351 | 32078 | 6:40713605 |
| 32079 | 6:40713909 | 32080 | 6:40714153 | 32081 | 6:40714938 | 32082 | 6:40715195 | 32083 | 6:40716811 |
| 32084 | 6:40721324 | 32087 | 6:40743678 | 32090 | 6:40770454 | 32091 | 6:40771992 | 32092 | 6:40771993 |
| 32186 | 6:41827472 | 32240 | 6:42833384 | 32249 | 6:42951534 | 32250 | 6:42951582 | 32251 | 6:42951695 |
| 32252 | 6:42951801 | 32253 | 6:42952447 | 32254 | 6:42953604 | 32255 | 6:42954090 | 32256 | 6:42956624 |
| 32257 | 6:42957873 | 32258 | 6:42961867 | 32259 | 6:42963490 | 32260 | 6:42966370 | 32261 | 6:42969572 |
| 32265 | 6:42996149 | 32268 | 6:43024569 | 32269 | 6:43035223 | 32270 | 6:43035520 | 32278 | 6:43178587 |
| 32292 | 6:43450785 | 32322 | 6:43878243 | 32328 | 6:43959607 | 32364 | 6:44686809 | 32417 | 6:44828139 |
| 32510 | 6:45452605 | 32511 | 6:45453963 | 32521 | 6:45702584 | 32555 | 6:46424441 | 32556 | 6:46446420 |
| 32564 | 6:46615384 | 32573 | 6:46883981 | 32575 | 6:46926732 | 32584 | 6:47108047 | 32611 | 6:47785922 |
| 32645 | 6:48798347 | 32726 | 6:50704099 | 32732 | 6:50885777 | 32742 | 6:51188886 | 32764 | 6:51982250 |
| 32769 | 6:52083644 | 32804 | 6:52427412 | 32805 | 6:52431484 | 32806 | 6:52434675 | 32807 | 6:52444382 |
| 32808 | 6:52444842 | 32809 | 6:52451412 | 32819 | 6:52675393 | 32832 | 6:53029143 | 32838 | 6:53151451 |
| 32907 | 6:55106382 | 32908 | 6:55121620 | 32912 | 6:55288741 | 32916 | 6:55337592 | 32921 | 6:55478053 |
| 32922 | 6:55478241 | 32923 | 6:55480385 | 32924 | 6:55481575 | 32925 | 6:55483221 | 32926 | 6:55483692 |
| 32927 | 6:55484196 | 32928 | 6:55484209 | 32929 | 6:55484776 | 32930 | 6:55484984 | 32931 | 6:55484996 |
| 32932 | 6:55485652 | 32933 | 6:55487306 | 32934 | 6:55487659 | 32935 | 6:55487698 | 32936 | 6:55487961 |
| 32937 | 6:55488137 | 32938 | 6:55488439 | 32939 | 6:55498328 | 32945 | 6:55534953 | 33154 | 6:60793645 |
| 33178 | 6:61401990 | 33201 | 6:62075988 | 33220 | 6:62746494 | 33287 | 6:64826176 | 33288 | 6:64860935 |
| 33301 | 6:65368807 | 33320 | 6:65902330 | 33385 | 6:68144658 | 33560 | 6:73271530 | 33568 | 6:73524311 |
| 33587 | 6:74274673 | 33588 | 6:74278129 | 33590 | 6:74325296 | 33595 | 6:74538501 | 33604 | 6:74934981 |
| 33638 | 6:76140833 | 33664 | 6:76838570 | 33669 | 6:76918617 | 33761 | 6:80397556 | 33786 | 6:80984642 |
| 33856 | 6:83284076 | 33857 | 6:83290251 | 33889 | 6:83810111 | 33919 | 6:84577409 | 33945 | 6:85170579 |
| 33958 | 6:85452102 | 33960 | 6:85473180 | 33973 | 6:85798206 | 33990 | 6:86146685 | 34015 | 6:86736214 |
| 34039 | 6:87076207 | 34076 | 6:87751257 | 34095 | 6:88150285 | 34146 | 6:89563672 | 34160 | 6:89750491 |
| 34167 | 6:89906046 | 34223 | 6:90999178 | 34252 | 6:91356781 | 34256 | 6:91397116 | 34311 | 6:92717918 |
| 34325 | 6:93027241 | 34355 | 6:93561742 | 34379 | 6:94081466 | 34380 | 6:94087064 | 34382 | 6:94089501 |
| 34383 | 6:94094573 | 34384 | 6:94096218 | 34385 | 6:94098014 | 34386 | 6:94098380 | 34387 | 6:94098998 |
| 34456 | 6:96190398 | 34499 | 6:97061854 | 34533 | 6:97481556 | 34534 | 6:97481830 | 34535 | 6:97482142 |
| 34536 | 6:97486160 | 34588 | 6:98499066 | 34597 | 6:98688262 | 34647 | 6:100180448 | 34648 | 6:100181539 |
| 34649 | 6:100181706 | 34650 | 6:100181746 | 34651 | 6:100182503 | 34652 | 6:100183086 | 34653 | 6:100183464 |
| 34654 | 6:100185002 | 34655 | 6:100185489 | 34656 | 6:100185949 | 34657 | 6:100186040 | 34658 | 6:100186295 |
| 34673 | 6:100520648 | 34698 | 6:101088719 | 34699 | 6:101089428 | 34765 | 6:102387484 | 34775 | 6:102527385 |
| 34820 | 6:103206312 | 34874 | 6:104209750 | 34913 | 6:105016409 | 34983 | 6:106924221 | 34985 | 6:106930321 |
| 34998 | 6:107232252 | 35017 | 6:107833335 | 35031 | 6:108314278 | 35032 | 6:108316551 | 35035 | 6:108336234 |
| 35040 | 6:108483986 | 35074 | 6:109282710 | 35086 | 6:109549186 | 35112 | 6:110036778 | 35120 | 6:110267946 |
| 35121 | 6:110296111 | 35154 | 6:111027360 | 35170 | 6:111251001 | 35181 | 6:111506757 | 35194 | 6:111752185 |
| 35195 | 6:111753497 | 35196 | 6:111755682 | 35203 | 6:111845284 | 35215 | 6:112274283 | 35225 | 6:112517961 |
| 35254 | 6:112951648 | 35258 | 6:113193897 | 35264 | 6:113278790 | 35269 | 6:113338198 | 35302 | 6:113832391 |
| 35304 | 6:113899018 | 35316 | 6:114099166 | 35317 | 6:114104514 | 35338 | 6:114493111 | 35356 | 6:114802066 |
| 35377 | 6:115232986 | 35397 | 6:115802755 | 35522 | 7:1173379 | 35548 | 7:1688750 | 35608 | 7:2637204 |
| 35647 | 7:3292376 | 35723 | 7:4848657 | 35745 | 7:5160131 | 35746 | 7:5169066 | 35754 | 7:5443639 |
| 35777 | 7:5627311 | 35779 | 7:5637555 | 35814 | 7:6109291 | 35815 | 7:6109369 | 35816 | 7:6110105 |
| 35817 | 7:6110223 | 35818 | 7:6110813 | 35819 | 7:6112482 | 35820 | 7:6112502 | 35821 | 7:6112503 |
| 35822 | 7:6112832 | 35824 | 7:6114838 | 35825 | 7:6118415 | 35826 | 7:6118766 | 35827 | 7:6119488 |
| 35828 | 7:6122989 | 35829 | 7:6123149 | 35830 | 7:6123438 | 35831 | 7:6128021 | 35832 | 7:6130590 |
| 35833 | 7:6130687 | 35834 | 7:6131292 | 35835 | 7:6131297 | 35836 | 7:6132484 | 35837 | 7:6132747 |
| 35838 | 7:6133083 | 35840 | 7:6137471 | 35854 | 7:6339944 | 35855 | 7:6341414 | 35856 | 7:6341470 |
| 35857 | 7:6341501 | 35858 | 7:6345398 | 35860 | 7:6348000 | 35861 | 7:6348353 | 35862 | 7:6352211 |
| 35863 | 7:6356100 | 35864 | 7:6356608 | 35865 | 7:6356670 | 35881 | 7:6710471 | 35894 | 7:6824241 |
| 35903 | 7:6999017 | 35914 | 7:7143257 | 35929 | 7:7557725 | 35930 | 7:7562490 | 35933 | 7:7584113 |
| 35959 | 7:8549050 | 35966 | 7:8914661 | 35977 | 7:9686485 | 35978 | 7:9760724 | 36006 | 7:11067502 |
| 36013 | 7:11123621 | 36031 | 7:11430993 | 36036 | 7:11668056 | 36046 | 7:11952515 | 36081 | 7:12499238 |
| 36157 | 7:13717835 | 36185 | 7:14182114 | 36243 | 7:15057296 | 36303 | 7:16284081 | 36325 | 7:16641516 |
| 36363 | 7:17636616 | 36365 | 7:17641071 | 36384 | 7:18214147 | 36409 | 7:18665015 | 36416 | 7:18849856 |
| 36433 | 7:19227351 | 36442 | 7:19317690 | 36444 | 7:19333133 | 36461 | 7:19721740 | 36516 | 7:20334637 |
| 36524 | 7:20395009 | 36536 | 7:20701671 | 36537 | 7:20704005 | 36539 | 7:20747147 | 36548 | 7:20935768 |
| 36639 | 7:23007875 | 36650 | 7:23297694 | 36651 | 7:23350564 | 36657 | 7:23528238 | 36658 | 7:23528885 |
| 36684 | 7:24067558 | 36692 | 7:24236323 | 36709 | 7:24746921 | 36734 | 7:25342888 | 36737 | 7:25403017 |
| 36843 | 7:27517669 | 36845 | 7:27577715 | 36846 | 7:27588151 | 36892 | 7:28685778 | 36910 | 7:29075991 |
| 36945 | 7:29836572 | 36952 | 7:29934618 | 36955 | 7:30045346 | 36969 | 7:30374597 | 36979 | 7:30662217 |
| 37046 | 7:31780219 | 37055 | 7:32039528 | 37090 | 7:33006143 | 37103 | 7:33314191 | 37140 | 7:34167737 |
| 37157 | 7:34561935 | 37166 | 7:34800911 | 37204 | 7:35829441 | 37224 | 7:36375051 | 37225 | 7:36381643 |
| 37271 | 7:37512158 | 37358 | 7:39431184 | 37383 | 7:40018615 | 37386 | 7:40105218 | 37387 | 7:40106188 |
| 37403 | 7:40313129 | 37421 | 7:40543374 | 37501 | 7:42717113 | 37502 | 7:42793836 | 37562 | 7:44383968 |
| 37577 | 7:44807464 | 37579 | 7:44874918 | 37589 | 7:45086131 | 37597 | 7:45264623 | 37598 | 7:45268714 |
| 37606 | 7:45462352 | 37628 | 7:46197379 | 37629 | 7:46207779 | 37647 | 7:46698742 | 37652 | 7:46763773 |
| 37665 | 7:47007606 | 37666 | 7:47009580 | 37667 | 7:47010093 | 37668 | 7:47010163 | 37669 | 7:47011323 |
| 37670 | 7:47011475 | 37671 | 7:47012085 | 37672 | 7:47012699 | 37674 | 7:47017310 | 37675 | 7:47018943 |
| 37676 | 7:47021031 | 37716 | 7:48370104 | 37736 | 7:48785206 | 37758 | 7:49338040 | 37807 | 7:51678528 |
| 37832 | 7:52217990 | 37892 | 7:52801631 | 37901 | 7:53051717 | 37903 | 7:53072922 | 37907 | 7:53192775 |
| 37929 | 7:53916384 | 37998 | 7:55634474 | 38004 | 7:55875367 | 38018 | 7:56132046 | 38036 | 7:56553298 |
| 38044 | 7:56873627 | 38072 | 7:57843543 | 38089 | 7:58267508 | 38091 | 7:58338126 | 38139 | 7:59331366 |
| 38146 | 7:59368692 | 38166 | 7:59579502 | 38249 | 7:61303310 | 38261 | 7:61485578 | 38264 | 7:61501849 |
| 38305 | 7:62532423 | 38306 | 7:62554501 | 38309 | 7:62558404 | 38377 | 7:63949067 | 38388 | 7:64285750 |
| 38397 | 7:64625175 | 38412 | 7:64930029 | 38413 | 7:64937658 | 38420 | 7:65072560 | 38432 | 7:65258702 |
| 38507 | 7:66460989 | 38538 | 7:67323191 | 38555 | 7:67781532 | 38557 | 7:67828291 | 38563 | 7:67940525 |
| 38586 | 7:68625153 | 38589 | 7:68641820 | 38642 | 7:69866274 | 38644 | 7:69879505 | 38646 | 7:69905767 |
| 38653 | 7:70037542 | 38655 | 7:70093469 | 38656 | 7:70099250 | 38666 | 7:70331724 | 38669 | 7:70419871 |
| 38689 | 7:71130508 | 38698 | 7:71415976 | 38716 | 7:71712692 | 38717 | 7:71727860 | 38722 | 7:71825182 |
| 38723 | 7:71830368 | 38725 | 7:71921897 | 38754 | 7:72753552 | 38794 | 7:74007645 | 38826 | 7:74850810 |
| 38869 | 7:75797751 | 38879 | 7:76006096 | 38880 | 7:76006417 | 38881 | 7:76010723 | 38916 | 7:76940482 |
| 38930 | 7:77150855 | 38941 | 7:77453724 | 38950 | 7:77656774 | 38964 | 7:77949425 | 38989 | 7:78379223 |
| 39000 | 7:78748906 | 39008 | 7:79016201 | 39009 | 7:79016267 | 39010 | 7:79016787 | 39011 | 7:79017056 |
| 39012 | 7:79017378 | 39013 | 7:79017385 | 39014 | 7:79017772 | 39015 | 7:79017959 | 39016 | 7:79018035 |
| 39017 | 7:79018571 | 39018 | 7:79018616 | 39019 | 7:79018620 | 39020 | 7:79019988 | 39021 | 7:79023682 |
| 39022 | 7:79025120 | 39023 | 7:79025266 | 39024 | 7:79041811 | 39025 | 7:79042476 | 39027 | 7:79048979 |
| 39028 | 7:79070660 | 39029 | 7:79071063 | 39031 | 7:79135260 | 39032 | 7:79135270 | 39040 | 7:79367716 |
| 39056 | 7:79660008 | 39076 | 7:80098520 | 39077 | 7:80104490 | 39096 | 7:80463366 | 39111 | 7:80573664 |
| 39122 | 7:80835359 | 39133 | 7:81097721 | 39221 | 7:82912666 | 39241 | 7:83502868 | 39259 | 7:83929718 |
| 39299 | 7:85098423 | 39310 | 7:85314414 | 39315 | 7:85586461 | 39383 | 7:87496719 | 39413 | 7:88462551 |
| 39425 | 7:88705722 | 39622 | 7:91895030 | 39706 | 7:94524953 | 39712 | 7:94671798 | 39713 | 7:94672003 |
| 39714 | 7:94688436 | 39715 | 7:94691325 | 39728 | 7:95018455 | 39768 | 7:95975870 | 39773 | 7:96088093 |
| 39774 | 7:96093182 | 39795 | 7:96175080 | 39796 | 7:96175132 | 39797 | 7:96175168 | 39798 | 7:96175341 |
| 39799 | 7:96175357 | 39800 | 7:96175409 | 39801 | 7:96175451 | 39802 | 7:96175628 | 39803 | 7:96176143 |
| 39804 | 7:96176528 | 39805 | 7:96176908 | 39809 | 7:96193901 | 39812 | 7:96199226 | 39815 | 7:96205718 |
| 39816 | 7:96205942 | 39817 | 7:96206884 | 39818 | 7:96207458 | 39819 | 7:96207882 | 39820 | 7:96208060 |
| 39821 | 7:96208106 | 39822 | 7:96208582 | 39823 | 7:96208995 | 39824 | 7:96209096 | 39825 | 7:96209862 |
| 39826 | 7:96211592 | 39827 | 7:96211829 | 39828 | 7:96216996 | 39832 | 7:96221555 | 39833 | 7:96223919 |
| 39834 | 7:96228261 | 39835 | 7:96228951 | 39836 | 7:96231447 | 39837 | 7:96235988 | 39838 | 7:96236371 |
| 39839 | 7:96236926 | 39840 | 7:96239015 | 39841 | 7:96239023 | 39842 | 7:96242837 | 39843 | 7:96246776 |
| 39844 | 7:96246937 | 39845 | 7:96248619 | 39846 | 7:96248835 | 39847 | 7:96249753 | 39848 | 7:96249787 |
| 39849 | 7:96249862 | 39850 | 7:96254816 | 39851 | 7:96263665 | 39852 | 7:96264122 | 39853 | 7:96264141 |
| 39854 | 7:96265124 | 39855 | 7:96265144 | 39856 | 7:96265161 | 39857 | 7:96265371 | 39858 | 7:96265379 |
| 39860 | 7:96271576 | 39861 | 7:96271653 | 39862 | 7:96273181 | 39863 | 7:96273246 | 39864 | 7:96273247 |
| 39865 | 7:96273562 | 39866 | 7:96273613 | 39867 | 7:96274725 | 39869 | 7:96291284 | 39870 | 7:96291379 |
| 39871 | 7:96291389 | 39872 | 7:96291500 | 39873 | 7:96291530 | 39874 | 7:96293330 | 39875 | 7:96293559 |
| 39876 | 7:96293758 | 39877 | 7:96293861 | 39878 | 7:96295220 | 39879 | 7:96295518 | 39880 | 7:96295778 |
| 39881 | 7:96295878 | 39882 | 7:96295906 | 39883 | 7:96295937 | 39884 | 7:96296075 | 39885 | 7:96296087 |
| 39886 | 7:96296169 | 39887 | 7:96296233 | 39888 | 7:96296513 | 39889 | 7:96296531 | 39890 | 7:96296546 |
| 39891 | 7:96296683 | 39892 | 7:96297463 | 39893 | 7:96297555 | 39894 | 7:96297857 | 39895 | 7:96298677 |
| 39896 | 7:96299647 | 39897 | 7:96300501 | 39898 | 7:96303921 | 39899 | 7:96304204 | 39900 | 7:96305341 |
| 39901 | 7:96305698 | 39902 | 7:96307964 | 39903 | 7:96308750 | 39904 | 7:96308945 | 39905 | 7:96309033 |
| 39906 | 7:96310180 | 39907 | 7:96311062 | 39909 | 7:96314531 | 39910 | 7:96314540 | 39913 | 7:96318724 |
| 39914 | 7:96322543 | 39915 | 7:96322732 | 39916 | 7:96323877 | 39917 | 7:96323928 | 39918 | 7:96324360 |
| 39919 | 7:96330432 | 39920 | 7:96331236 | 39921 | 7:96331603 | 39922 | 7:96333195 | 39923 | 7:96333277 |
| 39924 | 7:96333341 | 39925 | 7:96333563 | 39926 | 7:96333575 | 39927 | 7:96334610 | 39928 | 7:96334702 |
| 39929 | 7:96334763 | 39930 | 7:96335706 | 39931 | 7:96337614 | 39933 | 7:96354116 | 39935 | 7:96355876 |
| 39936 | 7:96358329 | 39937 | 7:96361391 | 39938 | 7:96363115 | 39940 | 7:96374561 | 39949 | 7:96519372 |
| 39953 | 7:96606759 | 40022 | 7:98399080 | 40043 | 7:99244131 | 40046 | 7:99254092 | 40085 | 7:100105110 |
| 40106 | 7:100563689 | 40108 | 7:100578900 | 40112 | 7:100659403 | 40127 | 7:101215525 | 40166 | 7:102357637 |
| 40201 | 7:103052550 | 40216 | 7:103329082 | 40224 | 7:103487103 | 40229 | 7:103675594 | 40232 | 7:103729325 |
| 40239 | 7:103982179 | 40240 | 7:103982180 | 40241 | 7:103982288 | 40242 | 7:103982423 | 40243 | 7:103985377 |
| 40253 | 7:104230973 | 40291 | 7:105373160 | 40294 | 7:105399311 | 40296 | 7:105442182 | 40297 | 7:105515412 |
| 40324 | 7:106146247 | 40372 | 7:107094209 | 40415 | 7:108495274 | 40496 | 8:356371 | 40500 | 8:395127 |
| 40585 | 8:2113854 | 40598 | 8:2387478 | 40600 | 8:2420577 | 40637 | 8:3508161 | 40640 | 8:3527178 |
| 40645 | 8:3640550 | 40647 | 8:3666778 | 40718 | 8:5496138 | 40732 | 8:5839173 | 40736 | 8:5906023 |
| 40751 | 8:6327904 | 40804 | 8:7593827 | 40861 | 8:8914108 | 40865 | 8:9015228 | 40882 | 8:9392626 |
| 40960 | 8:11244122 | 40966 | 8:11330242 | 40968 | 8:11403633 | 40975 | 8:11522513 | 40984 | 8:11668581 |
| 40991 | 8:11856184 | 40997 | 8:12018134 | 41009 | 8:12363854 | 41050 | 8:13811430 | 41095 | 8:15117985 |
| 41099 | 8:15188506 | 41110 | 8:15477698 | 41112 | 8:15494200 | 41118 | 8:15553158 | 41119 | 8:15574036 |
| 41126 | 8:15701758 | 41137 | 8:16168316 | 41138 | 8:16168775 | 41201 | 8:17444173 | 41211 | 8:17638060 |
| 41227 | 8:18138392 | 41250 | 8:18931382 | 41279 | 8:19741400 | 41290 | 8:19953078 | 41331 | 8:20945241 |
| 41343 | 8:21351226 | 41403 | 8:23473039 | 41410 | 8:23722262 | 41451 | 8:24658012 | 41460 | 8:24730121 |
| 41567 | 8:25718774 | 41578 | 8:25952516 | 41581 | 8:26020143 | 41607 | 8:26647199 | 41617 | 8:26902337 |
| 41618 | 8:26902597 | 41619 | 8:26902692 | 41620 | 8:26906751 | 41621 | 8:26909158 | 41622 | 8:26909371 |
| 41663 | 8:28392311 | 41694 | 8:29260025 | 41703 | 8:29463858 | 41797 | 8:31800228 | 41798 | 8:31843921 |
| 41829 | 8:32784569 | 41840 | 8:33342641 | 41853 | 8:33651679 | 41868 | 8:34125341 | 41876 | 8:34286689 |
| 41883 | 8:34490228 | 41897 | 8:34815302 | 41907 | 8:35172469 | 41963 | 8:36234097 | 41982 | 8:36725916 |
| 41989 | 8:36958536 | 42015 | 8:37563030 | 42052 | 8:38794675 | 42072 | 8:39672661 | 42134 | 8:40784197 |
| 42138 | 8:40885929 | 42141 | 8:41023065 | 42185 | 8:42062463 | 42259 | 8:44042188 | 42291 | 8:45099245 |
| 42293 | 8:45139392 | 42318 | 8:45729304 | 42324 | 8:45797263 | 42335 | 8:46074143 | 42336 | 8:46102241 |
| 42337 | 8:46116001 | 42338 | 8:46116354 | 42339 | 8:46135520 | 42341 | 8:46137990 | 42342 | 8:46144578 |
| 42344 | 8:46153342 | 42345 | 8:46156573 | 42375 | 8:46982936 | 42420 | 8:48031781 | 42430 | 8:48479991 |
| 42539 | 8:51426634 | 42545 | 8:51576820 | 42575 | 8:52252692 | 42585 | 8:52524895 | 42602 | 8:52976427 |
| 42613 | 8:53298802 | 42616 | 8:53382084 | 42702 | 8:55675444 | 42724 | 8:56364736 | 42725 | 8:56366787 |
| 42751 | 8:56934422 | 42753 | 8:56941050 | 42754 | 8:57019107 | 42783 | 8:57597865 | 42810 | 8:58618980 |
| 42832 | 8:59315871 | 42871 | 8:60125622 | 42934 | 8:61718348 | 42938 | 8:61889487 | 42971 | 8:62566075 |
| 42972 | 8:62584738 | 42992 | 8:63183224 | 43052 | 8:64941395 | 43080 | 8:65506614 | 43107 | 8:66205907 |
| 43118 | 8:66471868 | 43138 | 8:66967439 | 43144 | 8:67016183 | 43176 | 8:67787815 | 43178 | 8:67793980 |
| 43188 | 8:67954514 | 43191 | 8:68086072 | 43206 | 8:68512363 | 43207 | 8:68517559 | 43214 | 8:68740750 |
| 43231 | 8:69106674 | 43234 | 8:69118901 | 43266 | 8:70045486 | 43271 | 8:70141611 | 43282 | 8:70418020 |
| 43313 | 8:71060104 | 43314 | 8:71090353 | 43325 | 8:71516540 | 43350 | 8:72260036 | 43357 | 8:72432728 |
| 43358 | 8:72437508 | 43365 | 8:72557656 | 43388 | 8:73136540 | 43419 | 8:73847683 | 43451 | 8:74692744 |
| 43498 | 8:76193941 | 43517 | 8:76534193 | 43528 | 8:76736188 | 43587 | 8:78346120 | 43592 | 8:78443361 |
| 43628 | 8:79886867 | 43672 | 8:81022175 | 43675 | 8:81050611 | 43723 | 8:82076199 | 43741 | 8:82402914 |
| 43744 | 8:82410990 | 43754 | 8:82684234 | 43775 | 8:83245271 | 43786 | 8:83673295 | 43800 | 8:83846337 |
| 43806 | 8:83935130 | 43889 | 8:86085819 | 43893 | 8:86142301 | 43906 | 8:86539317 | 43913 | 8:86746201 |
| 43924 | 8:86977892 | 43928 | 8:87127829 | 43929 | 8:87139627 | 43930 | 8:87141766 | 43931 | 8:87142536 |
| 43933 | 8:87168896 | 43934 | 8:87175091 | 43935 | 8:87177155 | 43936 | 8:87183548 | 43937 | 8:87200658 |
| 43938 | 8:87218682 | 43946 | 8:87439832 | 44026 | 8:89300462 | 44066 | 8:90333835 | 44083 | 8:90850582 |
| 44093 | 8:90959304 | 44126 | 8:91835974 | 44162 | 8:92493864 | 44184 | 8:93235330 | 44287 | 8:96565770 |
| 44307 | 8:97085820 | 44344 | 8:98025826 | 44346 | 8:98099120 | 44377 | 8:98925263 | 44383 | 8:99073097 |
| 44444 | 8:100321613 | 44447 | 8:100390505 | 44521 | 8:102187006 | 44544 | 8:102423408 | 44548 | 8:102462371 |
| 44563 | 8:102641248 | 44565 | 8:102643183 | 44575 | 8:102804781 | 44617 | 8:103596897 | 44665 | 8:104455217 |
| 44671 | 8:104552362 | 44698 | 8:105230189 | 44708 | 8:105413829 | 44750 | 8:106646055 | 44751 | 8:106656709 |
| 44775 | 8:107216243 | 44777 | 8:107231955 | 44791 | 8:107578002 | 44798 | 8:107830676 | 44819 | 8:108486026 |
| 44820 | 8:108568498 | 44825 | 8:108815568 | 44854 | 8:109956440 | 44867 | 8:110340060 | 44885 | 8:110872351 |
| 44889 | 8:110978542 | 44927 | 9:28156 | 44939 | 9:575925 | 44940 | 9:575959 | 44942 | 9:582062 |
| 44943 | 9:590791 | 44944 | 9:593091 | 44945 | 9:600069 | 44946 | 9:600393 | 44948 | 9:618129 |
| 44949 | 9:632185 | 44950 | 9:633412 | 44951 | 9:642243 | 44952 | 9:659431 | 44953 | 9:659947 |
| 44954 | 9:660978 | 44955 | 9:661495 | 44956 | 9:665906 | 44958 | 9:681361 | 44959 | 9:697171 |
| 44960 | 9:708137 | 44961 | 9:709297 | 44964 | 9:726333 | 44969 | 9:900985 | 45000 | 9:1570889 |
| 45010 | 9:1844977 | 45023 | 9:2164529 | 45053 | 9:3003204 | 45069 | 9:3571118 | 45103 | 9:4701519 |
| 45114 | 9:5085846 | 45144 | 9:5897942 | 45145 | 9:5911158 | 45148 | 9:6056118 | 45160 | 9:6416188 |
| 45193 | 9:7630506 | 45195 | 9:7644292 | 45210 | 9:7983021 | 45214 | 9:8111645 | 45256 | 9:9020374 |
| 45257 | 9:9029730 | 45403 | 9:11584044 | 45408 | 9:11752659 | 45448 | 9:12626683 | 45454 | 9:12671087 |
| 45455 | 9:12689678 | 45457 | 9:12695431 | 45458 | 9:12695934 | 45459 | 9:12695941 | 45460 | 9:12696018 |
| 45461 | 9:12696113 | 45462 | 9:12696251 | 45463 | 9:12696292 | 45464 | 9:12696440 | 45465 | 9:12696661 |
| 45466 | 9:12698815 | 45467 | 9:12698843 | 45468 | 9:12700184 | 45472 | 9:12770979 | 45479 | 9:13069374 |
| 45480 | 9:13082121 | 45482 | 9:13102812 | 45483 | 9:13103635 | 45484 | 9:13104067 | 45486 | 9:13108898 |
| 45487 | 9:13109246 | 45488 | 9:13109258 | 45489 | 9:13110088 | 45494 | 9:13242607 | 45533 | 9:14240906 |
| 45547 | 9:14661048 | 45581 | 9:15193969 | 45591 | 9:15445642 | 45614 | 9:15671238 | 45615 | 9:15672381 |
| 45616 | 9:15672435 | 45617 | 9:15672993 | 45618 | 9:15673156 | 45619 | 9:15673271 | 45620 | 9:15673402 |
| 45621 | 9:15674725 | 45632 | 9:15869515 | 45635 | 9:15935953 | 45639 | 9:15995486 | 45654 | 9:16430498 |
| 45663 | 9:16688073 | 45693 | 9:17598783 | 45694 | 9:17599118 | 45783 | 9:20470091 | 45787 | 9:20622642 |
| 45794 | 9:20764203 | 45820 | 9:21613566 | 45821 | 9:21636143 | 45822 | 9:21640871 | 45823 | 9:21643825 |
| 45825 | 9:21654717 | 45826 | 9:21683117 | 45827 | 9:21683679 | 45829 | 9:21715481 | 45830 | 9:21740090 |
| 45831 | 9:21757906 | 45855 | 9:22294337 | 45866 | 9:22593668 | 45869 | 9:22639958 | 45923 | 9:23824584 |
| 45927 | 9:23893279 | 45955 | 9:24560985 | 45994 | 9:25737610 | 45995 | 9:25738079 | 45996 | 9:25741377 |
| 46014 | 9:26124495 | 46025 | 9:26374532 | 46056 | 9:27294641 | 46078 | 9:27733478 | 46081 | 9:27779272 |
| 46083 | 9:27801667 | 46103 | 9:28362224 | 46104 | 9:28363030 | 46113 | 9:28567312 | 46114 | 9:28568804 |
| 46115 | 9:28569972 | 46116 | 9:28591418 | 46117 | 9:28593815 | 46122 | 9:28646308 | 46140 | 9:29091195 |
| 46143 | 9:29192292 | 46144 | 9:29204130 | 46145 | 9:29256950 | 46147 | 9:29283806 | 46148 | 9:29295585 |
| 46151 | 9:29380299 | 46152 | 9:29380683 | 46153 | 9:29381237 | 46154 | 9:29381255 | 46155 | 9:29383215 |
| 46156 | 9:29383228 | 46157 | 9:29383283 | 46158 | 9:29384303 | 46159 | 9:29384799 | 46160 | 9:29385786 |
| 46161 | 9:29385818 | 46162 | 9:29386059 | 46163 | 9:29386376 | 46164 | 9:29387098 | 46166 | 9:29400041 |
| 46167 | 9:29400182 | 46168 | 9:29402441 | 46169 | 9:29402650 | 46170 | 9:29403623 | 46171 | 9:29409271 |
| 46172 | 9:29411199 | 46187 | 9:29691912 | 46188 | 9:29692738 | 46199 | 9:30032893 | 46200 | 9:30033128 |
| 46218 | 9:30451686 | 46223 | 9:30612426 | 46225 | 9:30625188 | 46246 | 9:31013461 | 46311 | 9:32638638 |
| 46346 | 9:33419705 | 46367 | 9:33960698 | 46387 | 9:34556125 | 46410 | 9:35190440 | 46444 | 9:36105021 |
| 46494 | 9:37158301 | 46519 | 9:37870307 | 46552 | 9:38368263 | 46582 | 9:38867506 | 46583 | 9:38875675 |
| 46584 | 9:38878148 | 46586 | 9:38894269 | 46637 | 9:39995374 | 46695 | 9:41446116 | 46699 | 9:41532669 |
| 46739 | 9:42551675 | 46776 | 9:43626998 | 46784 | 9:43912263 | 46785 | 9:43915403 | 46798 | 9:44230197 |
| 46806 | 9:44371483 | 46812 | 9:44533430 | 46816 | 9:44595447 | 46859 | 9:46082473 | 46887 | 9:46922739 |
| 46902 | 9:47520187 | 46903 | 9:47553422 | 46907 | 9:47590125 | 46975 | 9:49674165 | 46989 | 9:50119411 |
| 47011 | 9:50615961 | 47022 | 9:50886609 | 47024 | 9:50944146 | 47064 | 9:52161063 | 47071 | 9:52377348 |
| 47106 | 9:53217250 | 47107 | 9:53217253 | 47108 | 9:53217815 | 47109 | 9:53217988 | 47110 | 9:53225739 |
| 47111 | 9:53226122 | 47112 | 9:53226145 | 47113 | 9:53227113 | 47114 | 9:53234782 | 47115 | 9:53245948 |
| 47116 | 9:53269708 | 47118 | 9:53277095 | 47119 | 9:53298978 | 47163 | 9:54707994 | 47167 | 9:54805840 |
| 47180 | 9:55233499 | 47206 | 9:56084986 | 47274 | 9:57805504 | 47290 | 9:58371472 | 47310 | 9:58992635 |
| 47323 | 9:59401110 | 47324 | 9:59402335 | 47327 | 9:59422003 | 47329 | 9:59459837 | 47373 | 9:60362161 |
| 47469 | 9:63010039 | 47474 | 9:63179604 | 47477 | 9:63266632 | 47524 | 9:64700448 | 47593 | 9:65489746 |
| 47601 | 9:65582850 | 47619 | 9:66094001 | 47639 | 9:66606853 | 47652 | 9:67044823 | 47659 | 9:67241727 |
| 47664 | 9:67366039 | 47677 | 9:67697561 | 47689 | 9:68036049 | 47693 | 9:68114580 | 47694 | 9:68115586 |
| 47713 | 9:68594580 | 47753 | 9:69635715 | 47757 | 9:69752727 | 47788 | 9:70415891 | 47795 | 9:70619172 |
| 47807 | 9:70987356 | 47808 | 9:70997161 | 47878 | 9:72597976 | 47889 | 9:72874182 | 47936 | 9:74030455 |
| 47949 | 9:74381423 | 47965 | 9:74748032 | 48044 | 9:76966345 | 48070 | 9:77485895 | 48073 | 9:77511368 |
| 48075 | 9:77523509 | 48078 | 9:77562662 | 48081 | 9:77694221 | 48083 | 9:77699388 | 48084 | 9:77713754 |
| 48085 | 9:77715782 | 48086 | 9:77716409 | 48087 | 9:77716687 | 48088 | 9:77717203 | 48089 | 9:77717466 |
| 48090 | 9:77718722 | 48092 | 9:77723623 | 48093 | 9:77728652 | 48094 | 9:77728956 | 48095 | 9:77729189 |
| 48096 | 9:77729397 | 48097 | 9:77729413 | 48098 | 9:77730079 | 48099 | 9:77730486 | 48100 | 9:77730739 |
| 48101 | 9:77730950 | 48102 | 9:77731339 | 48103 | 9:77731548 | 48104 | 9:77731587 | 48105 | 9:77731649 |
| 48106 | 9:77732824 | 48107 | 9:77733602 | 48108 | 9:77733985 | 48109 | 9:77734457 | 48110 | 9:77734537 |
| 48111 | 9:77734632 | 48112 | 9:77739942 | 48113 | 9:77740756 | 48114 | 9:77741688 | 48115 | 9:77742632 |
| 48116 | 9:77743840 | 48117 | 9:77744601 | 48118 | 9:77744620 | 48119 | 9:77745039 | 48120 | 9:77745584 |
| 48121 | 9:77745608 | 48122 | 9:77751329 | 48123 | 9:77751755 | 48124 | 9:77754808 | 48125 | 9:77756169 |
| 48126 | 9:77756446 | 48127 | 9:77756531 | 48128 | 9:77762645 | 48129 | 9:77764362 | 48132 | 9:77792668 |
| 48133 | 9:77804673 | 48134 | 9:77811111 | 48135 | 9:77811220 | 48136 | 9:77811579 | 48137 | 9:77811594 |
| 48138 | 9:77811612 | 48139 | 9:77811647 | 48140 | 9:77811795 | 48141 | 9:77812291 | 48142 | 9:77812385 |
| 48143 | 9:77812527 | 48144 | 9:77812542 | 48145 | 9:77812555 | 48146 | 9:77812585 | 48191 | 9:79533173 |
| 48196 | 9:79702375 | 48249 | 9:80821611 | 48264 | 9:81093450 | 48265 | 9:81096096 | 48282 | 9:81398472 |
| 48298 | 9:81743991 | 48302 | 9:81797320 | 48321 | 9:82185927 | 48326 | 9:82275767 | 48341 | 9:82639891 |
| 48346 | 9:82675266 | 48347 | 9:82681644 | 48349 | 9:82685499 | 48350 | 9:82696652 | 48351 | 9:82697299 |
| 48352 | 9:82699229 | 48353 | 9:82699333 | 48355 | 9:82706831 | 48356 | 9:82708646 | 48366 | 9:82981739 |
| 48375 | 9:83266367 | 48408 | 9:84082840 | 48409 | 9:84084042 | 48410 | 9:84084459 | 48411 | 9:84098275 |
| 48412 | 9:84100341 | 48413 | 9:84101062 | 48419 | 9:84280123 | 48430 | 9:84602199 | 48453 | 9:85206226 |
| 48527 | 9:86728832 | 48542 | 9:87041634 | 48554 | 9:87334131 | 48645 | 9:88942711 | 48697 | 9:89633025 |
| 48704 | 9:89813410 | 48724 | 9:90315206 | 48740 | 9:90776042 | 48765 | 9:91141962 | 48791 | 9:91739734 |
| 48805 | 9:92056243 | 48807 | 9:92137059 | 48974 | 9:95252173 | 48981 | 9:95442777 | 49012 | 9:96258094 |
| 49084 | 9:97052004 | 49086 | 9:97068291 | 49093 | 9:97195333 | 49115 | 9:97614153 | 49118 | 9:97651483 |
| 49158 | 9:98444344 | 49165 | 9:98565031 | 49183 | 9:99022247 | 49194 | 9:99214577 | 49227 | 9:99757934 |
| 49228 | 9:99766501 | 49286 | 9:100838600 | 49287 | 9:100865957 | 49301 | 9:101130910 | 49308 | 9:101233972 |
| 49316 | 9:101405391 | 49323 | 9:101487283 | 49399 | 9:102585986 | 49428 | 9:102905463 | 49443 | 9:103267046 |
| 49470 | 9:103818635 | 49557 | 10:1514567 | 49560 | 10:1608093 | 49571 | 10:1809005 | 49573 | 10:1812942 |
| 49575 | 10:1815628 | 49596 | 10:2181089 | 49600 | 10:2318770 | 49603 | 10:2355906 | 49633 | 10:2956009 |
| 49650 | 10:3286732 | 49685 | 10:3957621 | 49719 | 10:4637364 | 49756 | 10:5504897 | 49757 | 10:5506239 |
| 49758 | 10:5506479 | 49759 | 10:5506633 | 49760 | 10:5507158 | 49761 | 10:5507326 | 49762 | 10:5507349 |
| 49763 | 10:5507472 | 49764 | 10:5507500 | 49765 | 10:5507611 | 49766 | 10:5507931 | 49767 | 10:5507997 |
| 49768 | 10:5508125 | 49769 | 10:5508230 | 49770 | 10:5509205 | 49771 | 10:5509288 | 49772 | 10:5511487 |
| 49773 | 10:5511551 | 49774 | 10:5511726 | 49817 | 10:6459980 | 49835 | 10:6823353 | 49837 | 10:6940126 |
| 49899 | 10:8330854 | 49901 | 10:8385791 | 49917 | 10:8566513 | 49965 | 10:9790239 | 49972 | 10:9846509 |
| 49987 | 10:10261088 | 50046 | 10:11321931 | 50047 | 10:11322196 | 50076 | 10:12039647 | 50090 | 10:12326160 |
| 50091 | 10:12337486 | 50093 | 10:12394358 | 50099 | 10:12500777 | 50121 | 10:13059560 | 50124 | 10:13141850 |
| 50132 | 10:13207866 | 50133 | 10:13222364 | 50134 | 10:13224497 | 50135 | 10:13225359 | 50136 | 10:13226008 |
| 50139 | 10:13238081 | 50140 | 10:13248003 | 50142 | 10:13255208 | 50143 | 10:13258421 | 50144 | 10:13282620 |
| 50145 | 10:13286682 | 50149 | 10:13347534 | 50150 | 10:13347535 | 50179 | 10:13823541 | 50183 | 10:13888930 |
| 50189 | 10:13964255 | 50193 | 10:14079328 | 50212 | 10:14629981 | 50215 | 10:14653991 | 50257 | 10:15551922 |
| 50260 | 10:15625431 | 50298 | 10:16237750 | 50328 | 10:16751980 | 50350 | 10:17119224 | 50352 | 10:17147669 |
| 50367 | 10:17432966 | 50379 | 10:17732565 | 50396 | 10:18087252 | 50397 | 10:18089168 | 50398 | 10:18089791 |
| 50399 | 10:18089811 | 50400 | 10:18092432 | 50401 | 10:18095170 | 50402 | 10:18096798 | 50403 | 10:18100952 |
| 50404 | 10:18105123 | 50411 | 10:18246343 | 50458 | 10:19161271 | 50477 | 10:19610258 | 50508 | 10:20302691 |
| 50514 | 10:20365815 | 50579 | 10:21377001 | 50615 | 10:22113385 | 50620 | 10:22271591 | 50709 | 10:26380149 |
| 50714 | 10:26474079 | 50715 | 10:26474882 | 50741 | 10:26820943 | 50777 | 10:27459845 | 50848 | 10:28936817 |
| 50850 | 10:28939598 | 50861 | 10:29148690 | 50903 | 10:29990871 | 50973 | 10:32272335 | 50977 | 10:32347626 |
| 50980 | 10:32449180 | 50984 | 10:32556195 | 51013 | 10:33127535 | 51014 | 10:33187873 | 51029 | 10:33566653 |
| 51086 | 10:34900072 | 51089 | 10:34926072 | 51095 | 10:35028845 | 51152 | 10:36423193 | 51178 | 10:37020667 |
| 51199 | 10:37655975 | 51203 | 10:37686916 | 51241 | 10:38641559 | 51258 | 10:39024264 | 51342 | 10:41413651 |
| 51351 | 10:41730162 | 51352 | 10:41731424 | 51359 | 10:41898236 | 51375 | 10:42389264 | 51378 | 10:42430118 |
| 51380 | 10:42448787 | 51382 | 10:42452483 | 51383 | 10:42455131 | 51389 | 10:42580900 | 51404 | 10:43179077 |
| 51419 | 10:43825008 | 51479 | 10:45276327 | 51526 | 10:46079546 | 51575 | 10:47154407 | 51602 | 10:47651824 |
| 51624 | 10:48309146 | 51656 | 10:48956010 | 51683 | 10:49439054 | 51714 | 10:50089231 | 51803 | 10:52130146 |
| 51819 | 10:52557986 | 51853 | 10:53459983 | 51878 | 10:54196187 | 51893 | 10:54537058 | 51894 | 10:54542851 |
| 51931 | 10:55708254 | 51954 | 10:56299639 | 51978 | 10:56721716 | 51981 | 10:56843229 | 51982 | 10:56922663 |
| 51988 | 10:57043405 | 51991 | 10:57115514 | 52018 | 10:57976067 | 52081 | 10:59624803 | 52102 | 10:60442020 |
| 52108 | 10:60636369 | 52159 | 10:62050176 | 52174 | 10:62290589 | 52249 | 10:64233969 | 52267 | 10:64525088 |
| 52268 | 10:64536219 | 52269 | 10:64543768 | 52270 | 10:64548032 | 52271 | 10:64548906 | 52286 | 10:64979230 |
| 52295 | 10:65266598 | 52339 | 10:66097225 | 52340 | 10:66120437 | 52361 | 10:66674133 | 52362 | 10:66674580 |
| 52363 | 10:66675048 | 52364 | 10:66675130 | 52366 | 10:66770244 | 52422 | 10:68107692 | 52424 | 10:68140093 |
| 52444 | 10:68518650 | 52536 | 10:71021997 | 52557 | 10:71661492 | 52574 | 10:72103748 | 52600 | 10:73153480 |
| 52604 | 10:73217430 | 52615 | 10:73492848 | 52620 | 10:73620458 | 52681 | 10:75374747 | 52696 | 10:75774470 |
| 52699 | 10:75886187 | 52707 | 10:76141481 | 52708 | 10:76174432 | 52734 | 10:76609622 | 52735 | 10:76613357 |
| 52737 | 10:76718459 | 52746 | 10:76886184 | 52808 | 10:78222151 | 52816 | 10:78333529 | 52831 | 10:78834237 |
| 52838 | 10:78993174 | 52847 | 10:79256209 | 52855 | 10:79448706 | 52875 | 10:80033473 | 52893 | 10:80437796 |
| 52905 | 10:80755095 | 52952 | 10:81403359 | 53025 | 10:82946024 | 53076 | 10:84003158 | 53119 | 10:84929419 |
| 53136 | 10:85211692 | 53141 | 10:85308316 | 53146 | 10:85365819 | 53151 | 10:85506776 | 53153 | 10:85532563 |
| 53180 | 10:86322591 | 53190 | 10:86480002 | 53198 | 10:86564397 | 53234 | 10:87380593 | 53240 | 10:87570845 |
| 53303 | 10:88984928 | 53307 | 10:89076810 | 53328 | 10:89477438 | 53338 | 10:89716618 | 53376 | 10:90408131 |
| 53386 | 10:90606603 | 53387 | 10:90610286 | 53410 | 10:90869492 | 53412 | 10:90878413 | 53427 | 10:91270590 |
| 53500 | 10:92937814 | 53506 | 10:93026084 | 53510 | 10:93101284 | 53522 | 10:93436468 | 53527 | 10:93605955 |
| 53581 | 10:94985373 | 53592 | 10:95290678 | 53606 | 10:95624360 | 53629 | 10:96068605 | 53633 | 10:96193408 |
| 53649 | 10:96591095 | 53654 | 10:96786131 | 53655 | 10:96788386 | 53694 | 10:97467089 | 53707 | 10:97772610 |
| 53708 | 10:97775801 | 53709 | 10:97778298 | 53734 | 10:98439866 | 53759 | 10:98730218 | 53782 | 10:99444853 |
| 53785 | 10:99471490 | 53794 | 10:99596036 | 53811 | 10:99982401 | 53873 | 10:101139524 | 53898 | 10:101650412 |
| 53899 | 10:101660552 | 53900 | 10:101676024 | 53902 | 10:101703864 | 53916 | 10:101851706 | 53926 | 10:102065117 |
| 53928 | 10:102086827 | 53933 | 10:102132405 | 53939 | 10:102239500 | 53977 | 10:103152266 | 54020 | 11:779732 |
| 54030 | 11:923580 | 54035 | 11:1009994 | 54047 | 11:1211969 | 54118 | 11:2084360 | 54127 | 11:2256142 |
| 54133 | 11:2359945 | 54181 | 11:2799677 | 54196 | 11:3053638 | 54206 | 11:3153964 | 54238 | 11:3763768 |
| 54328 | 11:5718123 | 54345 | 11:6071587 | 54380 | 11:7143509 | 54398 | 11:7527861 | 54404 | 11:7599092 |
| 54441 | 11:8559109 | 54503 | 11:9819964 | 54512 | 11:10000902 | 54588 | 11:11731827 | 54594 | 11:11844298 |
| 54617 | 11:12454930 | 54653 | 11:13209044 | 54654 | 11:13222009 | 54689 | 11:13914152 | 54696 | 11:14200577 |
| 54727 | 11:15182386 | 54732 | 11:15285660 | 54749 | 11:15700826 | 54770 | 11:16056267 | 54804 | 11:16934584 |
| 54814 | 11:17117515 | 54867 | 11:18770128 | 54928 | 11:20656584 | 54943 | 11:21060821 | 54992 | 11:22018210 |
| 55000 | 11:22303794 | 55012 | 11:22569464 | 55020 | 11:22724476 | 55032 | 11:23083144 | 55068 | 11:23895849 |
| 55087 | 11:24338666 | 55097 | 11:24469097 | 55129 | 11:25273777 | 55140 | 11:25560609 | 55162 | 11:26154618 |
| 55165 | 11:26170393 | 55252 | 11:28239112 | 55304 | 11:29324448 | 55317 | 11:29530060 | 55324 | 11:29724736 |
| 55329 | 11:29759229 | 55355 | 11:30470011 | 55385 | 11:31068333 | 55434 | 11:32501555 | 55466 | 11:33302958 |
| 55503 | 11:34519584 | 55542 | 11:35824586 | 55552 | 11:36039319 | 55557 | 11:36151963 | 55562 | 11:36293996 |
| 55577 | 11:36723444 | 55599 | 11:37215399 | 55600 | 11:37240873 | 55626 | 11:37656828 | 55634 | 11:37841103 |
| 55637 | 11:37898906 | 55672 | 11:39016826 | 55701 | 11:40047215 | 55706 | 11:40222392 | 55716 | 11:40663028 |
| 55751 | 11:41311710 | 55752 | 11:41326914 | 55755 | 11:41328623 | 55758 | 11:41354523 | 55760 | 11:41356073 |
| 55766 | 11:41457760 | 55774 | 11:41843798 | 55796 | 11:42373804 | 55797 | 11:42374333 | 55798 | 11:42374619 |
| 55799 | 11:42374860 | 55800 | 11:42375434 | 55802 | 11:42378608 | 55803 | 11:42395653 | 55804 | 11:42395937 |
| 55808 | 11:42450574 | 55809 | 11:42458071 | 55826 | 11:42952869 | 55828 | 11:42977381 | 55834 | 11:43155397 |
| 55849 | 11:43447636 | 55855 | 11:43656266 | 55867 | 11:44164688 | 55871 | 11:44197049 | 55904 | 11:45053290 |
| 55950 | 11:46193646 | 55973 | 11:46703688 | 55976 | 11:46763745 | 55977 | 11:46763771 | 55978 | 11:46763772 |
| 55979 | 11:46763803 | 55980 | 11:46763967 | 55981 | 11:46764372 | 55982 | 11:46765309 | 55983 | 11:46769693 |
| 56013 | 11:47198478 | 56053 | 11:48210329 | 56122 | 11:48963797 | 56127 | 11:49061005 | 56128 | 11:49080138 |
| 56135 | 11:49179291 | 56138 | 11:49285957 | 56143 | 11:49404465 | 56147 | 11:49422373 | 56152 | 11:49575365 |
| 56153 | 11:49582523 | 56198 | 11:51041604 | 56201 | 11:51160828 | 56205 | 11:51255454 | 56206 | 11:51261576 |
| 56252 | 11:53012268 | 56253 | 11:53022205 | 56257 | 11:53214759 | 56272 | 11:53823211 | 56293 | 11:54411745 |
| 56301 | 11:54607804 | 56302 | 11:54611060 | 56303 | 11:54615185 | 56304 | 11:54619006 | 56305 | 11:54619299 |
| 56306 | 11:54621378 | 56307 | 11:54624272 | 56308 | 11:54625736 | 56309 | 11:54637534 | 56310 | 11:54640117 |
| 56311 | 11:54640249 | 56312 | 11:54644177 | 56313 | 11:54658429 | 56314 | 11:54667025 | 56315 | 11:54672210 |
| 56317 | 11:54687119 | 56318 | 11:54696136 | 56327 | 11:54925623 | 56341 | 11:55201147 | 56345 | 11:55322961 |
| 56384 | 11:56468221 | 56394 | 11:56809772 | 56395 | 11:56811610 | 56396 | 11:56812388 | 56408 | 11:57181716 |
| 56434 | 11:58004110 | 56441 | 11:58172817 | 56451 | 11:58552292 | 56452 | 11:58557598 | 56478 | 11:59155112 |
| 56498 | 11:59762890 | 56519 | 11:60453153 | 56528 | 11:60471884 | 56539 | 11:60491356 | 56609 | 11:62266387 |
| 56611 | 11:62305455 | 56612 | 11:62308757 | 56614 | 11:62379499 | 56617 | 11:62426969 | 56623 | 11:62609754 |
| 56681 | 11:63826838 | 56692 | 11:63976694 | 56707 | 11:64337696 | 56711 | 11:64464647 | 56737 | 11:65172790 |
| 56789 | 11:66422364 | 56887 | 11:68143046 | 56912 | 11:68896819 | 56918 | 11:69065684 | 56919 | 11:69065947 |
| 56920 | 11:69067967 | 56927 | 11:69141312 | 56934 | 11:69198908 | 56936 | 11:69213888 | 56965 | 11:69725037 |
| 56975 | 11:69945179 | 56979 | 11:70105382 | 56989 | 11:70270717 | 56991 | 11:70291428 | 57061 | 11:71167695 |
| 57063 | 11:71169475 | 57064 | 11:71171201 | 57065 | 11:71174044 | 57071 | 11:71187848 | 57083 | 11:71410081 |
| 57092 | 11:71596359 | 57093 | 11:71603815 | 57097 | 11:71735550 | 57118 | 11:72456335 | 57127 | 11:72799464 |
| 57171 | 11:73648343 | 57267 | 11:75692626 | 57288 | 11:76333610 | 57314 | 11:77013992 | 57318 | 11:77067925 |
| 57319 | 11:77077199 | 57320 | 11:77085172 | 57330 | 11:77232878 | 57340 | 11:77445226 | 57341 | 11:77485470 |
| 57396 | 11:78194981 | 57405 | 11:78326150 | 57555 | 11:81830178 | 57571 | 11:82150919 | 57595 | 11:82670314 |
| 57617 | 11:82895182 | 57620 | 11:82943647 | 57646 | 11:83482447 | 57648 | 11:83552676 | 57708 | 11:85258209 |
| 57718 | 11:85436750 | 57725 | 11:85653522 | 57730 | 11:85743559 | 57754 | 11:86206294 | 57755 | 11:86214871 |
| 57814 | 11:87707465 | 57847 | 11:87999059 | 57865 | 11:88032183 | 57884 | 11:88448012 | 57927 | 11:89546657 |
| 57946 | 11:89996070 | 57970 | 11:90503652 | 58023 | 11:91710069 | 58027 | 11:91791559 | 58028 | 11:91792786 |
| 58029 | 11:91796326 | 58030 | 11:91798254 | 58031 | 11:91807646 | 58032 | 11:91809950 | 58033 | 11:91809976 |
| 58042 | 11:92019924 | 58082 | 11:92823026 | 58091 | 11:93153121 | 58092 | 11:93165769 | 58093 | 11:93179602 |
| 58094 | 11:93185994 | 58100 | 11:93232642 | 58105 | 11:93261434 | 58106 | 11:93262306 | 58107 | 11:93262884 |
| 58108 | 11:93263825 | 58109 | 11:93279097 | 58111 | 11:93395136 | 58124 | 11:93858711 | 58143 | 11:94337656 |
| 58163 | 11:94860855 | 58168 | 11:95063641 | 58175 | 11:95296518 | 58268 | 11:97728887 | 58299 | 11:98222359 |
| 58303 | 11:98359510 | 58373 | 11:99755436 | 58492 | 11:102014618 | 58517 | 11:102577090 | 58524 | 11:102659659 |
| 58583 | 11:103383891 | 58596 | 11:103697414 | 58630 | 11:104473977 | 58644 | 11:104683228 | 58654 | 11:104829955 |
| 58656 | 11:104875554 | 58658 | 11:104927513 | 58661 | 11:104973365 | 58663 | 11:104974394 | 58681 | 11:105482462 |
| 58729 | 11:106793153 | 58731 | 11:106948591 | 58752 | 12:625880 | 58780 | 12:1498811 | 58821 | 12:2369495 |
| 58870 | 12:3405573 | 58875 | 12:3491002 | 58894 | 12:4026056 | 58925 | 12:4736478 | 58934 | 12:5016508 |
| 58960 | 12:5810001 | 59000 | 12:7239138 | 59032 | 12:8559085 | 59098 | 12:10166200 | 59115 | 12:10466559 |
| 59148 | 12:11055011 | 59172 | 12:11660529 | 59177 | 12:11743244 | 59237 | 12:13369400 | 59275 | 12:14073643 |
| 59284 | 12:14200873 | 59334 | 12:15275995 | 59404 | 12:16588996 | 59405 | 12:16617595 | 59445 | 12:17717003 |
| 59511 | 12:19127423 | 59512 | 12:19133184 | 59553 | 12:20149260 | 59563 | 12:20389759 | 59565 | 12:20491988 |
| 59566 | 12:20500610 | 59608 | 12:21574455 | 59700 | 12:23970417 | 59718 | 12:24380235 | 59730 | 12:24612900 |
| 59753 | 12:25107687 | 59760 | 12:25333357 | 59778 | 12:25727678 | 59779 | 12:25742060 | 59782 | 12:25755570 |
| 59787 | 12:25862626 | 59816 | 12:26904714 | 59834 | 12:27216744 | 59891 | 12:28914720 | 59952 | 12:30585785 |
| 59960 | 12:30737586 | 59964 | 12:30851151 | 59965 | 12:30851590 | 59978 | 12:31055012 | 60064 | 12:33121234 |
| 60068 | 12:33212754 | 60115 | 12:34706439 | 60158 | 12:35595816 | 60205 | 12:37118156 | 60252 | 12:39318432 |
| 60253 | 12:39337804 | 60254 | 12:39339971 | 60255 | 12:39375252 | 60295 | 12:40436881 | 60299 | 12:40699170 |
| 60310 | 12:41000477 | 60345 | 12:42446263 | 60346 | 12:42529266 | 60384 | 12:44349627 | 60414 | 12:45309570 |
| 60464 | 12:46902819 | 60503 | 12:47894827 | 60512 | 12:48234155 | 60524 | 12:48552141 | 60528 | 12:48621944 |
| 60557 | 12:49347591 | 60588 | 12:50012484 | 60654 | 12:51413041 | 60677 | 12:52057233 | 60712 | 12:52833730 |
| 60713 | 12:52841272 | 60714 | 12:52843772 | 60715 | 12:52859350 | 60717 | 12:52867407 | 60718 | 12:52868520 |
| 60720 | 12:52883259 | 60721 | 12:52888503 | 60736 | 12:53324225 | 60737 | 12:53326935 | 60748 | 12:53513237 |
| 60776 | 12:54070308 | 60778 | 12:54112016 | 60800 | 12:54537212 | 60823 | 12:55077299 | 60834 | 12:55543664 |
| 60864 | 12:56465958 | 60881 | 12:57046134 | 60949 | 12:59148039 | 60950 | 12:59163748 | 60958 | 12:59442298 |
| 61087 | 12:63768535 | 61092 | 12:63878306 | 61108 | 12:64352510 | 61118 | 12:64738377 | 61196 | 12:66786596 |
| 61240 | 12:67928955 | 61247 | 12:68098257 | 61248 | 12:68114447 | 61259 | 12:68270144 | 61275 | 12:68608809 |
| 61278 | 12:68710557 | 61290 | 12:68863135 | 61301 | 12:69019005 | 61309 | 12:69286353 | 61311 | 12:69340936 |
| 61318 | 12:69467691 | 61328 | 12:69553759 | 61332 | 12:69582989 | 61366 | 12:71235763 | 61382 | 12:71600338 |
| 61412 | 12:72958303 | 61413 | 12:72961991 | 61446 | 12:73518731 | 61482 | 12:74197569 | 61497 | 12:74496338 |
| 61522 | 12:75101487 | 61529 | 12:75242221 | 61532 | 12:75277582 | 61537 | 12:75375680 | 61590 | 12:76682892 |
| 61618 | 12:77393088 | 61638 | 12:77847200 | 61645 | 12:77993107 | 61673 | 12:78662420 | 61704 | 12:79284630 |
| 61718 | 12:79542780 | 61728 | 12:79738869 | 61749 | 12:80221064 | 61750 | 12:80223568 | 61751 | 12:80224696 |
| 61755 | 12:80311844 | 61760 | 12:80405755 | 61762 | 12:80523810 | 61818 | 12:81778398 | 61836 | 12:82156928 |
| 61850 | 12:82530033 | 61865 | 12:82796759 | 61888 | 12:83319494 | 61922 | 12:84042281 | 61956 | 12:84892891 |
| 61968 | 12:85033607 | 61980 | 12:85234076 | 61998 | 12:85446719 | 62009 | 12:85641586 | 62011 | 12:85752928 |
| 62060 | 12:86779904 | 62103 | 13:648927 | 62135 | 13:1384950 | 62142 | 13:1583266 | 62183 | 13:2254372 |
| 62213 | 13:2708064 | 62260 | 13:3861084 | 62336 | 13:5664259 | 62337 | 13:5666787 | 62361 | 13:6275220 |
| 62366 | 13:6361450 | 62408 | 13:7483031 | 62413 | 13:7556779 | 62434 | 13:8333741 | 62438 | 13:8415584 |
| 62443 | 13:8529126 | 62444 | 13:8533157 | 62445 | 13:8537080 | 62446 | 13:8542772 | 62450 | 13:8682255 |
| 62451 | 13:8682878 | 62452 | 13:8683708 | 62453 | 13:8685414 | 62454 | 13:8686278 | 62524 | 13:10400290 |
| 62526 | 13:10426820 | 62537 | 13:10575199 | 62553 | 13:11047542 | 62573 | 13:12140250 | 62613 | 13:13240348 |
| 62654 | 13:14380630 | 62665 | 13:14649538 | 62669 | 13:14768979 | 62675 | 13:14878218 | 62690 | 13:15298554 |
| 62693 | 13:15509230 | 62707 | 13:15907128 | 62845 | 13:18649928 | 62849 | 13:18704268 | 62880 | 13:18741567 |
| 62886 | 13:18795212 | 62893 | 13:18926634 | 62904 | 13:19113938 | 62927 | 13:19611692 | 62953 | 13:19991782 |
| 62955 | 13:20085636 | 62962 | 13:20299025 | 62985 | 13:20633696 | 62991 | 13:20728403 | 62999 | 13:21031730 |
| 63014 | 13:21160488 | 63016 | 13:21199075 | 63039 | 13:21675803 | 63072 | 13:22491633 | 63080 | 13:22645333 |
| 63142 | 13:24652712 | 63154 | 13:24964331 | 63170 | 13:25313389 | 63233 | 13:26864651 | 63296 | 13:28352469 |
| 63310 | 13:28677951 | 63311 | 13:28716131 | 63313 | 13:28719397 | 63314 | 13:28721486 | 63315 | 13:28726672 |
| 63328 | 13:29164280 | 63331 | 13:29203677 | 63409 | 13:30734642 | 63413 | 13:30835419 | 63417 | 13:30928296 |
| 63433 | 13:31170980 | 63479 | 13:32132576 | 63480 | 13:32133958 | 63481 | 13:32143886 | 63482 | 13:32152278 |
| 63494 | 13:32274709 | 63506 | 13:32489566 | 63521 | 13:32651702 | 63522 | 13:32653621 | 63523 | 13:32654103 |
| 63528 | 13:32846791 | 63529 | 13:32846825 | 63530 | 13:32847172 | 63531 | 13:32847400 | 63542 | 13:33084958 |
| 63543 | 13:33086348 | 63572 | 13:33880293 | 63599 | 13:34800113 | 63619 | 13:35179138 | 63622 | 13:35197458 |
| 63766 | 13:38636130 | 63767 | 13:38662619 | 63768 | 13:38676202 | 63769 | 13:38680784 | 63775 | 13:38810147 |
| 63781 | 13:38839160 | 63782 | 13:38893419 | 63786 | 13:38949069 | 63796 | 13:39090329 | 63800 | 13:39121053 |
| 63832 | 13:39696126 | 63852 | 13:40100334 | 63860 | 13:40220102 | 63870 | 13:40453192 | 63872 | 13:40473511 |
| 63873 | 13:40486594 | 63874 | 13:40486661 | 63940 | 13:42071486 | 63972 | 13:42587452 | 63982 | 13:42606861 |
| 64003 | 13:42825481 | 64024 | 13:43516130 | 64032 | 13:43689434 | 64033 | 13:43726105 | 64039 | 13:43774352 |
| 64059 | 13:44057830 | 64060 | 13:44115215 | 64105 | 13:44933952 | 64122 | 13:44991547 | 64157 | 13:45403608 |
| 64176 | 13:45936548 | 64210 | 13:46658312 | 64216 | 13:46758989 | 64245 | 13:47366925 | 64250 | 13:47472066 |
| 64254 | 13:47495500 | 64264 | 13:47827146 | 64284 | 13:48238821 | 64285 | 13:48240137 | 64286 | 13:48240290 |
| 64287 | 13:48240322 | 64288 | 13:48240351 | 64289 | 13:48241221 | 64290 | 13:48241373 | 64291 | 13:48241390 |
| 64292 | 13:48243603 | 64293 | 13:48245013 | 64294 | 13:48245053 | 64296 | 13:48269729 | 64297 | 13:48274446 |
| 64318 | 13:49199089 | 64319 | 13:49199105 | 64320 | 13:49200231 | 64419 | 13:52063310 | 64429 | 13:52466434 |
| 64496 | 13:53368735 | 64499 | 13:53503066 | 64516 | 13:54246719 | 64522 | 13:54512037 | 64533 | 13:54732415 |
| 64593 | 13:56026065 | 64649 | 13:57529999 | 64650 | 13:57541123 | 64669 | 13:58019158 | 64698 | 13:58633062 |
| 64708 | 13:58899200 | 64721 | 13:59016374 | 64732 | 13:59248087 | 64735 | 13:59289762 | 64766 | 13:60057972 |
| 64776 | 13:60212933 | 64798 | 13:60664451 | 64807 | 13:60980294 | 64822 | 13:61522788 | 64846 | 13:62118304 |
| 64852 | 13:62277677 | 64888 | 13:62739693 | 64889 | 13:62752247 | 64893 | 13:62800301 | 64899 | 13:62843052 |
| 64946 | 13:63972761 | 64967 | 13:64523897 | 64988 | 13:65061778 | 65005 | 13:65480528 | 65011 | 13:65604335 |
| 65014 | 13:65656406 | 65021 | 13:65714427 | 65023 | 13:65715682 | 65033 | 13:65806803 | 65052 | 13:66249739 |
| 65070 | 13:66636049 | 65076 | 13:66754479 | 65077 | 13:66811790 | 65082 | 13:66930495 | 65099 | 13:67376538 |
| 65112 | 13:67740112 | 65114 | 13:67768319 | 65118 | 13:67910634 | 65141 | 13:68545291 | 65177 | 13:69533712 |
| 65193 | 13:69946188 | 65219 | 13:70559101 | 65254 | 13:71281225 | 65255 | 13:71297993 | 65264 | 13:71463110 |
| 65285 | 13:71937940 | 65291 | 13:72156689 | 65345 | 13:73285435 | 65394 | 13:74233601 | 65400 | 13:74368669 |
| 65402 | 13:74389287 | 65435 | 13:74875799 | 65458 | 13:75539335 | 65459 | 13:75546891 | 65460 | 13:75550544 |
| 65475 | 13:75773530 | 65490 | 13:76048819 | 65495 | 13:76206814 | 65497 | 13:76256932 | 65513 | 13:76540633 |
| 65533 | 13:76870141 | 65610 | 13:78594569 | 65632 | 13:78943793 | 65642 | 13:79095868 | 65658 | 13:79307341 |
| 65667 | 13:79411595 | 65668 | 13:79427565 | 65687 | 13:79896093 | 65693 | 13:80009218 | 65699 | 13:80110062 |
| 65700 | 13:80110693 | 65701 | 13:80112377 | 65702 | 13:80112537 | 65703 | 13:80112873 | 65704 | 13:80113035 |
| 65705 | 13:80113195 | 65706 | 13:80119481 | 65707 | 13:80120615 | 65708 | 13:80121232 | 65709 | 13:80121378 |
| 65710 | 13:80121492 | 65711 | 13:80121527 | 65712 | 13:80122117 | 65713 | 13:80123053 | 65714 | 13:80123496 |
| 65715 | 13:80123540 | 65716 | 13:80128525 | 65717 | 13:80128577 | 65718 | 13:80128592 | 65719 | 13:80128724 |
| 65720 | 13:80133414 | 65726 | 13:80284611 | 65730 | 13:80353010 | 65731 | 13:80354577 | 65741 | 13:80564883 |
| 65792 | 13:81650045 | 65813 | 13:82057542 | 65814 | 13:82060563 | 65847 | 13:82692047 | 65937 | 14:1748354 |
| 65939 | 14:1780705 | 65960 | 14:2169492 | 65962 | 14:2196270 | 65979 | 14:2821082 | 65999 | 14:3180090 |
| 66000 | 14:3180092 | 66001 | 14:3184037 | 66003 | 14:3188747 | 66031 | 14:3341005 | 66057 | 14:3777934 |
| 66059 | 14:3807311 | 66065 | 14:3937653 | 66096 | 14:4438274 | 66107 | 14:4826114 | 66109 | 14:4845905 |
| 66112 | 14:4900819 | 66114 | 14:4924180 | 66123 | 14:5079999 | 66141 | 14:5424566 | 66143 | 14:5442736 |
| 66144 | 14:5442915 | 66155 | 14:5642922 | 66161 | 14:5745989 | 66162 | 14:5746008 | 66163 | 14:5746079 |
| 66164 | 14:5746088 | 66167 | 14:5767279 | 66168 | 14:5767316 | 66182 | 14:6051710 | 66222 | 14:6580835 |
| 66251 | 14:7127015 | 66255 | 14:7190153 | 66260 | 14:7315224 | 66267 | 14:7446316 | 66296 | 14:7973769 |
| 66298 | 14:7981735 | 66299 | 14:7984017 | 66301 | 14:7993501 | 66303 | 14:8000324 | 66306 | 14:8006167 |
| 66307 | 14:8008471 | 66309 | 14:8029440 | 66324 | 14:8315882 | 66339 | 14:8555196 | 66342 | 14:8578594 |
| 66401 | 14:9370788 | 66424 | 14:9883880 | 66445 | 14:10330270 | 66487 | 14:11125973 | 66519 | 14:11675980 |
| 66520 | 14:11687647 | 66521 | 14:11700111 | 66536 | 14:12017220 | 66572 | 14:12683380 | 66573 | 14:12683869 |
| 66575 | 14:12685152 | 66576 | 14:12685153 | 66580 | 14:12718898 | 66581 | 14:12726976 | 66582 | 14:12726981 |
| 66642 | 14:14433818 | 66674 | 14:15281409 | 66683 | 14:15476637 | 66707 | 14:15871072 | 66740 | 14:16464011 |
| 66800 | 14:17774684 | 66848 | 14:18880176 | 66911 | 14:20615962 | 66917 | 14:20673221 | 66920 | 14:20723405 |
| 66921 | 14:20728338 | 66939 | 14:21119658 | 66940 | 14:21124340 | 66973 | 14:21853648 | 67075 | 14:22718406 |
| 67158 | 14:23199725 | 67159 | 14:23200416 | 67160 | 14:23200440 | 67161 | 14:23202111 | 67162 | 14:23202373 |
| 67163 | 14:23202574 | 67164 | 14:23202642 | 67165 | 14:23202665 | 67166 | 14:23205217 | 67167 | 14:23206165 |
| 67168 | 14:23209278 | 67169 | 14:23210217 | 67170 | 14:23211671 | 67171 | 14:23212276 | 67172 | 14:23212424 |
| 67174 | 14:23216603 | 67176 | 14:23216967 | 67177 | 14:23217298 | 67178 | 14:23217676 | 67179 | 14:23217682 |
| 67182 | 14:23217984 | 67184 | 14:23219378 | 67185 | 14:23220306 | 67186 | 14:23221060 | 67187 | 14:23221105 |
| 67188 | 14:23221660 | 67190 | 14:23223448 | 67192 | 14:23232920 | 67293 | 14:23296927 | 67353 | 14:23590382 |
| 67532 | 14:25538462 | 67551 | 14:25555299 | 67553 | 14:25557838 | 67562 | 14:25875465 | 67572 | 14:26025356 |
| 67592 | 14:26183095 | 67615 | 14:26313111 | 67631 | 14:26716130 | 67638 | 14:26937569 | 67647 | 14:27145363 |
| 67659 | 14:27578987 | 67666 | 14:27804049 | 67669 | 14:27925661 | 67680 | 14:28283881 | 67684 | 14:28475888 |
| 67701 | 14:28805553 | 67713 | 14:29204640 | 67728 | 14:29506987 | 67737 | 14:29651348 | 67751 | 14:30011410 |
| 67787 | 14:30531165 | 67806 | 14:31151661 | 67810 | 14:31229071 | 67812 | 14:31276668 | 67813 | 14:31348707 |
| 67839 | 14:32042320 | 67840 | 14:32080892 | 67847 | 14:32295836 | 67848 | 14:32298088 | 67849 | 14:32298121 |
| 67850 | 14:32298404 | 67851 | 14:32298532 | 67852 | 14:32298550 | 67853 | 14:32298592 | 67854 | 14:32298623 |
| 67855 | 14:32298715 | 67856 | 14:32298807 | 67857 | 14:32298889 | 67858 | 14:32298929 | 67859 | 14:32298949 |
| 67860 | 14:32298983 | 67861 | 14:32301242 | 67862 | 14:32301629 | 67863 | 14:32301804 | 67880 | 14:32768091 |
| 67902 | 14:33283287 | 67914 | 14:33481134 | 67945 | 14:34107991 | 67963 | 14:34616797 | 67966 | 14:34703575 |
| 67968 | 14:34726901 | 68040 | 14:36456652 | 68066 | 14:37335063 | 68131 | 14:39204823 | 68180 | 14:40564678 |
| 68185 | 14:40761575 | 68199 | 14:41245865 | 68207 | 14:41477789 | 68211 | 14:41608564 | 68246 | 14:42371257 |
| 68252 | 14:42499551 | 68275 | 14:43217965 | 68314 | 14:44597633 | 68367 | 14:45598918 | 68386 | 14:45953548 |
| 68393 | 14:46043440 | 68412 | 14:46302755 | 68430 | 14:46746014 | 68435 | 14:46846924 | 68463 | 14:47562082 |
| 68479 | 14:48154116 | 68495 | 14:48422440 | 68496 | 14:48424765 | 68507 | 14:48722429 | 68578 | 14:50964252 |
| 68611 | 14:51741282 | 68659 | 14:53067901 | 68716 | 14:54352030 | 68717 | 14:54359397 | 68727 | 14:54470262 |
| 68733 | 14:54629600 | 68785 | 14:56030241 | 68869 | 14:58169542 | 68876 | 14:58268988 | 68879 | 14:58347367 |
| 68898 | 14:58795728 | 68902 | 14:58882362 | 68914 | 14:59072940 | 68931 | 14:59422421 | 68945 | 14:59890123 |
| 68960 | 14:60186718 | 68998 | 14:61242862 | 69030 | 14:61805601 | 69069 | 14:62451453 | 69075 | 14:62584372 |
| 69079 | 14:62700380 | 69092 | 14:62957699 | 69106 | 14:63240134 | 69117 | 14:63473862 | 69160 | 14:64610567 |
| 69174 | 14:65146328 | 69215 | 14:66160998 | 69219 | 14:66259689 | 69228 | 14:66384994 | 69232 | 14:66419660 |
| 69326 | 14:68515393 | 69354 | 14:69351733 | 69370 | 14:69913790 | 69382 | 14:70082152 | 69391 | 14:70201845 |
| 69400 | 14:70384514 | 69408 | 14:70595169 | 69415 | 14:70881552 | 69416 | 14:70890232 | 69427 | 14:71123353 |
| 69443 | 14:71687136 | 69472 | 14:72410713 | 69494 | 14:72897923 | 69506 | 14:73212493 | 69515 | 14:73427877 |
| 69517 | 14:73468390 | 69534 | 14:73985016 | 69570 | 14:75029436 | 69571 | 14:75034802 | 69572 | 14:75035607 |
| 69573 | 14:75035827 | 69574 | 14:75035857 | 69575 | 14:75035923 | 69608 | 14:76029716 | 69631 | 14:76258289 |
| 69633 | 14:76268094 | 69680 | 14:77464768 | 69686 | 14:77542458 | 69690 | 14:77708662 | 69700 | 14:77954720 |
| 69736 | 14:78637209 | 69737 | 14:78644200 | 69742 | 14:78699024 | 69750 | 14:78929919 | 69762 | 14:79335372 |
| 69767 | 14:79486373 | 69783 | 14:79820967 | 69790 | 14:79974645 | 69805 | 14:80236030 | 69819 | 14:80591048 |
| 69864 | 14:81465207 | 69901 | 14:82003511 | 70000 | 15:2555400 | 70037 | 15:3651481 | 70043 | 15:3796804 |
| 70044 | 15:3859257 | 70045 | 15:3861053 | 70046 | 15:3861177 | 70048 | 15:3872949 | 70049 | 15:3874148 |
| 70050 | 15:3874413 | 70051 | 15:3875123 | 70052 | 15:3875291 | 70053 | 15:3875480 | 70054 | 15:3875800 |
| 70055 | 15:3876161 | 70056 | 15:3876211 | 70057 | 15:3876259 | 70058 | 15:3884992 | 70059 | 15:3885455 |
| 70060 | 15:3888242 | 70061 | 15:3888270 | 70070 | 15:4006347 | 70080 | 15:4274305 | 70107 | 15:4817619 |
| 70108 | 15:4821833 | 70109 | 15:4827554 | 70110 | 15:4850527 | 70111 | 15:4855973 | 70113 | 15:4859691 |
| 70114 | 15:4874530 | 70132 | 15:5345428 | 70133 | 15:5345893 | 70134 | 15:5346513 | 70135 | 15:5346544 |
| 70136 | 15:5347645 | 70137 | 15:5348421 | 70138 | 15:5348505 | 70139 | 15:5348800 | 70140 | 15:5348852 |
| 70141 | 15:5348875 | 70142 | 15:5349143 | 70143 | 15:5349849 | 70144 | 15:5350256 | 70145 | 15:5350323 |
| 70146 | 15:5350355 | 70147 | 15:5353059 | 70148 | 15:5353099 | 70149 | 15:5355811 | 70150 | 15:5355909 |
| 70151 | 15:5356157 | 70152 | 15:5356186 | 70153 | 15:5356365 | 70154 | 15:5356372 | 70155 | 15:5357215 |
| 70156 | 15:5358118 | 70157 | 15:5358230 | 70158 | 15:5358668 | 70159 | 15:5359593 | 70160 | 15:5359865 |
| 70161 | 15:5359868 | 70162 | 15:5361660 | 70163 | 15:5361661 | 70164 | 15:5365576 | 70165 | 15:5380375 |
| 70166 | 15:5381250 | 70167 | 15:5382096 | 70168 | 15:5383165 | 70169 | 15:5383541 | 70170 | 15:5384183 |
| 70171 | 15:5384848 | 70172 | 15:5384963 | 70173 | 15:5384997 | 70174 | 15:5385062 | 70175 | 15:5385270 |
| 70176 | 15:5385290 | 70177 | 15:5385312 | 70178 | 15:5385429 | 70179 | 15:5386627 | 70180 | 15:5386633 |
| 70181 | 15:5386655 | 70182 | 15:5386721 | 70183 | 15:5387773 | 70184 | 15:5387850 | 70185 | 15:5388162 |
| 70186 | 15:5388165 | 70187 | 15:5388635 | 70188 | 15:5388661 | 70189 | 15:5388748 | 70190 | 15:5389424 |
| 70191 | 15:5389588 | 70192 | 15:5389616 | 70193 | 15:5389859 | 70194 | 15:5389978 | 70195 | 15:5390296 |
| 70196 | 15:5390320 | 70197 | 15:5390450 | 70198 | 15:5390553 | 70199 | 15:5390863 | 70200 | 15:5390921 |
| 70201 | 15:5391192 | 70202 | 15:5391298 | 70203 | 15:5391508 | 70204 | 15:5391682 | 70205 | 15:5391799 |
| 70206 | 15:5392452 | 70207 | 15:5392859 | 70208 | 15:5392923 | 70209 | 15:5393194 | 70210 | 15:5393401 |
| 70211 | 15:5393671 | 70212 | 15:5393753 | 70213 | 15:5394283 | 70214 | 15:5395547 | 70215 | 15:5395610 |
| 70216 | 15:5395743 | 70217 | 15:5395751 | 70218 | 15:5395855 | 70219 | 15:5395947 | 70220 | 15:5398109 |
| 70221 | 15:5398341 | 70222 | 15:5398346 | 70223 | 15:5398513 | 70224 | 15:5398559 | 70225 | 15:5400626 |
| 70226 | 15:5401120 | 70227 | 15:5401525 | 70228 | 15:5401536 | 70229 | 15:5401568 | 70230 | 15:5401820 |
| 70231 | 15:5401853 | 70232 | 15:5404746 | 70233 | 15:5404825 | 70234 | 15:5405498 | 70235 | 15:5405505 |
| 70236 | 15:5406067 | 70237 | 15:5406105 | 70238 | 15:5406213 | 70239 | 15:5406470 | 70240 | 15:5406961 |
| 70241 | 15:5407075 | 70242 | 15:5407142 | 70243 | 15:5407235 | 70244 | 15:5407350 | 70245 | 15:5407539 |
| 70246 | 15:5407760 | 70247 | 15:5407841 | 70248 | 15:5407895 | 70249 | 15:5408434 | 70250 | 15:5408552 |
| 70251 | 15:5408640 | 70252 | 15:5408900 | 70253 | 15:5409527 | 70254 | 15:5409532 | 70255 | 15:5409637 |
| 70256 | 15:5409728 | 70257 | 15:5409914 | 70258 | 15:5410082 | 70259 | 15:5410166 | 70260 | 15:5410173 |
| 70261 | 15:5410337 | 70262 | 15:5410342 | 70263 | 15:5410429 | 70264 | 15:5410813 | 70265 | 15:5410848 |
| 70266 | 15:5412771 | 70267 | 15:5412796 | 70268 | 15:5412835 | 70269 | 15:5412869 | 70270 | 15:5413090 |
| 70271 | 15:5413111 | 70272 | 15:5413650 | 70273 | 15:5415034 | 70274 | 15:5415477 | 70275 | 15:5416298 |
| 70276 | 15:5416324 | 70277 | 15:5417129 | 70278 | 15:5417450 | 70279 | 15:5417663 | 70280 | 15:5418542 |
| 70284 | 15:5503948 | 70303 | 15:6004286 | 70319 | 15:6435496 | 70337 | 15:7003211 | 70338 | 15:7008452 |
| 70340 | 15:7019231 | 70341 | 15:7029657 | 70342 | 15:7033643 | 70348 | 15:7265163 | 70349 | 15:7265165 |
| 70350 | 15:7265341 | 70351 | 15:7265425 | 70352 | 15:7265838 | 70353 | 15:7270627 | 70354 | 15:7270650 |
| 70355 | 15:7272155 | 70356 | 15:7272347 | 70357 | 15:7272651 | 70358 | 15:7275537 | 70359 | 15:7275789 |
| 70360 | 15:7275947 | 70361 | 15:7276254 | 70362 | 15:7277640 | 70363 | 15:7277661 | 70364 | 15:7278217 |
| 70411 | 15:8492046 | 70427 | 15:9014277 | 70429 | 15:9031126 | 70432 | 15:9102618 | 70438 | 15:9169770 |
| 70439 | 15:9233443 | 70471 | 15:10068806 | 70472 | 15:10075553 | 70474 | 15:10079990 | 70475 | 15:10080765 |
| 70476 | 15:10081644 | 70511 | 15:11396612 | 70557 | 15:13243732 | 70596 | 15:14363124 | 70609 | 15:14575841 |
| 70610 | 15:14594338 | 70629 | 15:15205635 | 70630 | 15:15239992 | 70707 | 15:16376589 | 70708 | 15:16439066 |
| 70735 | 15:17085252 | 70738 | 15:17150531 | 70745 | 15:17184652 | 70765 | 15:17609663 | 70797 | 15:18443747 |
| 70805 | 15:18738117 | 70819 | 15:19107499 | 70839 | 15:19655124 | 70859 | 15:20139691 | 70869 | 15:20440646 |
| 70890 | 15:20723501 | 70945 | 15:22143335 | 70975 | 15:22778474 | 70976 | 15:22778563 | 70977 | 15:22779768 |
| 70984 | 15:22928839 | 70990 | 15:23127718 | 70993 | 15:23186412 | 71073 | 15:24244322 | 71098 | 15:24733535 |
| 71100 | 15:24758009 | 71125 | 15:25277078 | 71134 | 15:25472864 | 71147 | 15:25791312 | 71158 | 15:25955785 |
| 71179 | 15:26356076 | 71196 | 15:26746546 | 71208 | 15:27116571 | 71241 | 15:27939885 | 71250 | 15:28057977 |
| 71255 | 15:28119260 | 71281 | 15:28579106 | 71361 | 15:30204684 | 71411 | 15:31423842 | 71480 | 15:32908026 |
| 71484 | 15:32949060 | 71520 | 15:33787224 | 71547 | 15:34234555 | 71557 | 15:34409813 | 71575 | 15:34768501 |
| 71595 | 15:35125952 | 71629 | 15:36004891 | 71630 | 15:36008307 | 71631 | 15:36026492 | 71648 | 15:36401889 |
| 71659 | 15:36632711 | 71691 | 15:37336930 | 71694 | 15:37400581 | 71695 | 15:37400733 | 71696 | 15:37402318 |
| 71697 | 15:37402363 | 71698 | 15:37402682 | 71699 | 15:37403163 | 71700 | 15:37405162 | 71701 | 15:37406604 |
| 71702 | 15:37408508 | 71726 | 15:38124244 | 71729 | 15:38238774 | 71747 | 15:38627437 | 71754 | 15:38756627 |
| 71765 | 15:39027306 | 71774 | 15:39310122 | 71779 | 15:39360198 | 71788 | 15:39612413 | 71831 | 15:40620626 |
| 71848 | 15:41008572 | 71872 | 15:41577758 | 71873 | 15:41586379 | 71894 | 15:41925488 | 71937 | 15:42825209 |
| 71945 | 15:42913923 | 71990 | 15:43478979 | 72010 | 15:43778894 | 72015 | 15:43842403 | 72025 | 15:43940904 |
| 72031 | 15:44171870 | 72055 | 15:44625080 | 72119 | 15:46158689 | 72132 | 15:46492762 | 72164 | 15:46744389 |
| 72165 | 15:46777715 | 72167 | 15:46785999 | 72168 | 15:46790581 | 72169 | 15:46804156 | 72170 | 15:46852906 |
| 72171 | 15:46854891 | 72175 | 15:46873315 | 72219 | 15:47951518 | 72223 | 15:48016378 | 72242 | 15:48211767 |
| 72259 | 15:48584212 | 72288 | 15:49705651 | 72400 | 15:52067358 | 72432 | 15:52906987 | 72450 | 15:53308730 |
| 72455 | 15:53312462 | 72514 | 15:54171179 | 72571 | 15:55496596 | 72580 | 15:55679447 | 72584 | 15:55794756 |
| 72629 | 15:56617358 | 72704 | 15:57721772 | 72710 | 15:57822336 | 72753 | 15:58596845 | 72786 | 15:59497090 |
| 72810 | 15:60299723 | 72821 | 15:60544990 | 72828 | 15:60665356 | 72838 | 15:61116189 | 72841 | 15:61144336 |
| 72844 | 15:61187610 | 72847 | 15:61230749 | 72848 | 15:61235200 | 72883 | 15:61807140 | 72897 | 15:62087105 |
| 72904 | 15:62399301 | 72913 | 15:62562520 | 72945 | 15:63169519 | 72956 | 15:63484094 | 72978 | 15:63866432 |
| 72980 | 15:63956384 | 72984 | 15:63969563 | 73010 | 15:64570035 | 73011 | 15:64573599 | 73012 | 15:64581068 |
| 73013 | 15:64581832 | 73014 | 15:64581860 | 73015 | 15:64587577 | 73016 | 15:64587694 | 73017 | 15:64587726 |
| 73018 | 15:64587727 | 73019 | 15:64587894 | 73032 | 15:64805974 | 73034 | 15:64822114 | 73036 | 15:64828957 |
| 73037 | 15:64829763 | 73055 | 15:65133696 | 73057 | 15:65176452 | 73068 | 15:65338322 | 73110 | 15:66198995 |
| 73133 | 15:66626921 | 73136 | 15:66654107 | 73137 | 15:66659219 | 73157 | 15:67252520 | 73183 | 15:67982185 |
| 73232 | 15:69401513 | 73297 | 15:71439330 | 73316 | 15:71924774 | 73318 | 15:71954992 | 73320 | 15:71975292 |
| 73324 | 15:72049215 | 73432 | 15:74395188 | 73433 | 15:74409395 | 73453 | 15:74803075 | 73462 | 15:75064377 |
| 73463 | 15:75065891 | 73464 | 15:75071311 | 73469 | 15:75183095 | 73471 | 15:75207042 | 73478 | 15:75433003 |
| 73581 | 15:77653525 | 73585 | 15:77702400 | 73588 | 15:77785862 | 73593 | 15:77886217 | 73647 | 15:78764970 |
| 73658 | 15:79089495 | 73691 | 15:80200231 | 73697 | 15:80308461 | 73708 | 15:80425465 | 73780 | 15:81982872 |
| 73833 | 15:83017252 | 73857 | 15:83501029 | 73858 | 15:83507518 | 73859 | 15:83514913 | 73860 | 15:83519848 |
| 73861 | 15:83524365 | 73863 | 15:83529608 | 73885 | 15:84090905 | 73971 | 16:1756551 | 74018 | 16:2807132 |
| 74022 | 16:2880813 | 74046 | 16:3278886 | 74067 | 16:3645905 | 74074 | 16:3832226 | 74078 | 16:3983617 |
| 74085 | 16:4041201 | 74113 | 16:4472306 | 74116 | 16:4488724 | 74134 | 16:5011968 | 74222 | 16:7875660 |
| 74227 | 16:8061836 | 74247 | 16:8833166 | 74264 | 16:9480615 | 74299 | 16:10761697 | 74367 | 16:12564264 |
| 74370 | 16:12610513 | 74376 | 16:12712264 | 74380 | 16:12809176 | 74416 | 16:13500639 | 74493 | 16:15596092 |
| 74509 | 16:16114051 | 74543 | 16:17341478 | 74577 | 16:18497037 | 74680 | 16:21073857 | 74697 | 16:21482215 |
| 74705 | 16:21676658 | 74716 | 16:22006522 | 74717 | 16:22052486 | 74721 | 16:22158028 | 74731 | 16:22445521 |
| 74735 | 16:22522151 | 74736 | 16:22527368 | 74784 | 16:23721556 | 74795 | 16:23949095 | 74810 | 16:24332233 |
| 74873 | 16:25194415 | 74886 | 16:25230576 | 74895 | 16:25259731 | 74896 | 16:25267299 | 74912 | 16:25869035 |
| 74914 | 16:25919271 | 74922 | 16:26068625 | 74962 | 16:26927299 | 75005 | 16:28019627 | 75093 | 16:30731403 |
| 75115 | 16:31224822 | 75116 | 16:31245895 | 75135 | 16:31715125 | 75141 | 16:31812156 | 75192 | 16:33108294 |
| 75193 | 16:33145311 | 75196 | 16:33256074 | 75208 | 16:33505042 | 75221 | 16:33889094 | 75227 | 16:34042467 |
| 75228 | 16:34043548 | 75232 | 16:34106781 | 75248 | 16:34589155 | 75329 | 16:36525688 | 75352 | 16:37160322 |
| 75435 | 16:39120749 | 75447 | 16:39536783 | 75474 | 16:40415228 | 75484 | 16:40686277 | 75519 | 16:41883664 |
| 75545 | 16:42004893 | 75548 | 16:42055479 | 75562 | 16:42802530 | 75565 | 16:42843655 | 75640 | 16:46097209 |
| 75644 | 16:46156095 | 75677 | 16:46962612 | 75715 | 16:47752936 | 75724 | 16:48024037 | 75725 | 16:48025956 |
| 75726 | 16:48033049 | 75727 | 16:48034263 | 75729 | 16:48067867 | 75734 | 16:48186793 | 75735 | 16:48187331 |
| 75736 | 16:48187489 | 75737 | 16:48223403 | 75738 | 16:48230117 | 75739 | 16:48232464 | 75751 | 16:48528203 |
| 75754 | 16:48566545 | 75755 | 16:48591914 | 75771 | 16:48864424 | 75776 | 16:49014011 | 75786 | 16:49264493 |
| 75852 | 16:51207748 | 75916 | 16:52579557 | 76051 | 16:56128576 | 76058 | 16:56235648 | 76083 | 16:56653049 |
| 76110 | 16:57538081 | 76150 | 16:58399171 | 76164 | 16:58621735 | 76178 | 16:58963242 | 76182 | 16:59073877 |
| 76184 | 16:59075986 | 76190 | 16:59245993 | 76192 | 16:59265813 | 76193 | 16:59266170 | 76200 | 16:59328729 |
| 76205 | 16:59411723 | 76226 | 16:60040160 | 76243 | 16:60336822 | 76259 | 16:60792834 | 76289 | 16:61435887 |
| 76345 | 16:62739421 | 76365 | 16:63159271 | 76368 | 16:63219362 | 76383 | 16:63498892 | 76385 | 16:63554652 |
| 76398 | 16:63842771 | 76400 | 16:63858162 | 76411 | 16:64136818 | 76424 | 16:64408574 | 76435 | 16:64563350 |
| 76440 | 16:64632519 | 76442 | 16:64637819 | 76443 | 16:64637824 | 76444 | 16:64637826 | 76453 | 16:64811600 |
| 76524 | 16:66273725 | 76529 | 16:66428578 | 76542 | 16:66625242 | 76552 | 16:66834448 | 76585 | 16:67467822 |
| 76587 | 16:67495360 | 76590 | 16:67506416 | 76593 | 16:67552727 | 76603 | 16:67685441 | 76635 | 16:68623796 |
| 76640 | 16:68718917 | 76642 | 16:68746747 | 76646 | 16:68890172 | 76655 | 16:69079413 | 76691 | 16:69777086 |
| 76693 | 16:69813037 | 76701 | 16:70045391 | 76717 | 16:70339833 | 76729 | 16:70576773 | 76748 | 16:70946593 |
| 76774 | 16:71760780 | 76788 | 16:72102560 | 76825 | 16:72662418 | 76830 | 16:72687591 | 76842 | 16:72970330 |
| 76861 | 16:73473194 | 76864 | 16:73513959 | 76895 | 16:73873564 | 76951 | 16:75067479 | 76972 | 16:75350380 |
| 76990 | 16:75714163 | 77032 | 16:76538771 | 77064 | 16:77153817 | 77099 | 16:77965851 | 77102 | 16:78019437 |
| 77132 | 16:78710736 | 77152 | 16:78992583 | 77166 | 16:79102626 | 77238 | 16:80715002 | 77241 | 16:80886197 |
| 77246 | 17:111106 | 77250 | 17:171222 | 77251 | 17:172400 | 77253 | 17:305126 | 77262 | 17:486383 |
| 77374 | 17:3335273 | 77379 | 17:3441488 | 77422 | 17:4260319 | 77443 | 17:4584704 | 77446 | 17:4621398 |
| 77452 | 17:4717447 | 77469 | 17:5097696 | 77470 | 17:5097857 | 77489 | 17:5622055 | 77510 | 17:6077823 |
| 77557 | 17:6872378 | 77579 | 17:7317589 | 77580 | 17:7338578 | 77582 | 17:7342145 | 77586 | 17:7478372 |
| 77606 | 17:7876357 | 77608 | 17:7883935 | 77619 | 17:8132536 | 77638 | 17:8600944 | 77642 | 17:8668787 |
| 77704 | 17:10109146 | 77716 | 17:10263523 | 77725 | 17:10597515 | 77742 | 17:11173860 | 77747 | 17:11293045 |
| 77753 | 17:11395090 | 77762 | 17:11714108 | 77763 | 17:11715058 | 77770 | 17:11817054 | 77787 | 17:12195499 |
| 77788 | 17:12195568 | 77789 | 17:12195662 | 77790 | 17:12196276 | 77791 | 17:12196708 | 77805 | 17:12486159 |
| 77808 | 17:12628635 | 77906 | 17:14816935 | 77909 | 17:14863506 | 77931 | 17:15328888 | 77949 | 17:15663502 |
| 77958 | 17:15910083 | 77975 | 17:16368625 | 78009 | 17:17274244 | 78010 | 17:17279868 | 78011 | 17:17280788 |
| 78012 | 17:17280990 | 78013 | 17:17281225 | 78024 | 17:17411908 | 78039 | 17:17775173 | 78082 | 17:18818243 |
| 78086 | 17:18869685 | 78105 | 17:19223651 | 78106 | 17:19245275 | 78109 | 17:19292348 | 78110 | 17:19295355 |
| 78111 | 17:19295356 | 78112 | 17:19295499 | 78113 | 17:19295504 | 78133 | 17:19689334 | 78153 | 17:20218072 |
| 78173 | 17:20920601 | 78177 | 17:21089322 | 78195 | 17:21650017 | 78203 | 17:21824364 | 78259 | 17:24078894 |
| 78278 | 17:24557966 | 78308 | 17:25485693 | 78320 | 17:25835294 | 78333 | 17:26208169 | 78336 | 17:26275689 |
| 78338 | 17:26313864 | 78347 | 17:26586857 | 78355 | 17:26669513 | 78379 | 17:27150917 | 78417 | 17:28123761 |
| 78429 | 17:28336908 | 78457 | 17:29248479 | 78514 | 17:30796206 | 78543 | 17:31545528 | 78556 | 17:31865010 |
| 78558 | 17:31877966 | 78579 | 17:32478146 | 78583 | 17:32532692 | 78596 | 17:32813643 | 78625 | 17:33559323 |
| 78633 | 17:33630931 | 78661 | 17:34398154 | 78696 | 17:35542521 | 78722 | 17:36254116 | 78747 | 17:37178207 |
| 78773 | 17:38080956 | 78783 | 17:38278802 | 78789 | 17:38562984 | 78797 | 17:38814783 | 78824 | 17:39676479 |
| 78825 | 17:39677255 | 78826 | 17:39679094 | 78832 | 17:39827471 | 78833 | 17:39828115 | 78834 | 17:39828525 |
| 78836 | 17:39840626 | 78838 | 17:39849566 | 78839 | 17:39854144 | 78840 | 17:39858951 | 78843 | 17:39882893 |
| 78844 | 17:39883222 | 78845 | 17:39885556 | 78846 | 17:39886164 | 78847 | 17:39887756 | 78848 | 17:39893701 |
| 78849 | 17:39894975 | 78850 | 17:39896327 | 78851 | 17:39896972 | 78877 | 17:40559838 | 78880 | 17:40569673 |
| 78881 | 17:40569857 | 78882 | 17:40570434 | 78885 | 17:40597210 | 78889 | 17:40687599 | 78891 | 17:40702874 |
| 78893 | 17:40727620 | 78924 | 17:41582600 | 78993 | 17:43272612 | 78996 | 17:43327605 | 79034 | 17:44279469 |
| 79079 | 17:45388796 | 79090 | 17:45567473 | 79117 | 17:46182697 | 79173 | 17:47495174 | 79183 | 17:47822968 |
| 79184 | 17:47826553 | 79203 | 17:48325513 | 79216 | 17:48653000 | 79227 | 17:48892420 | 79233 | 17:49128208 |
| 79234 | 17:49131387 | 79272 | 17:49921719 | 79309 | 17:50714394 | 79310 | 17:50720459 | 79311 | 17:50722838 |
| 79312 | 17:50723471 | 79313 | 17:50727878 | 79314 | 17:50730200 | 79315 | 17:50731825 | 79317 | 17:50814958 |
| 79357 | 17:51667657 | 79381 | 17:52060992 | 79385 | 17:52081616 | 79386 | 17:52082865 | 79387 | 17:52087837 |
| 79388 | 17:52087901 | 79424 | 17:53278122 | 79462 | 17:54135006 | 79538 | 17:56024288 | 79550 | 17:56299499 |
| 79555 | 17:56435818 | 79572 | 17:57052924 | 79586 | 17:57342574 | 79605 | 17:57757425 | 79607 | 17:57774071 |
| 79611 | 17:57814505 | 79628 | 17:58146798 | 79636 | 17:58314278 | 79637 | 17:58317198 | 79638 | 17:58321497 |
| 79639 | 17:58322279 | 79640 | 17:58323397 | 79641 | 17:58324863 | 79642 | 17:58325379 | 79643 | 17:58325583 |
| 79644 | 17:58325642 | 79645 | 17:58325662 | 79646 | 17:58325692 | 79647 | 17:58325868 | 79648 | 17:58325926 |
| 79649 | 17:58326148 | 79650 | 17:58330227 | 79651 | 17:58331401 | 79653 | 17:58334167 | 79654 | 17:58335083 |
| 79655 | 17:58335597 | 79656 | 17:58337576 | 79657 | 17:58338335 | 79681 | 17:59019311 | 79699 | 17:59378547 |
| 79701 | 17:59392366 | 79712 | 17:59683258 | 79747 | 17:60329105 | 79749 | 17:60358707 | 79757 | 17:60444808 |
| 79798 | 17:61041921 | 79820 | 17:61336784 | 79824 | 17:61432513 | 79831 | 17:61511820 | 79893 | 17:63198489 |
| 79977 | 17:64661316 | 79989 | 17:64973779 | 79994 | 17:65038744 | 80026 | 17:65697499 | 80041 | 17:65972365 |
| 80051 | 17:66165602 | 80052 | 17:66169310 | 80053 | 17:66170354 | 80054 | 17:66171241 | 80055 | 17:66171742 |
| 80056 | 17:66172830 | 80071 | 17:66374177 | 80113 | 17:67428489 | 80128 | 17:67720645 | 80131 | 17:67842378 |
| 80139 | 17:68113867 | 80189 | 17:69059007 | 80213 | 17:69580013 | 80223 | 17:69757640 | 80227 | 17:69882555 |
| 80246 | 17:70318401 | 80276 | 17:70659081 | 80282 | 17:70929780 | 80318 | 17:71539730 | 80327 | 17:71761848 |
| 80331 | 17:71853596 | 80332 | 17:71855169 | 80333 | 17:71859405 | 80335 | 17:71900536 | 80406 | 18:712517 |
| 80420 | 18:990363 | 80449 | 18:1484040 | 80458 | 18:1637197 | 80473 | 18:1743436 | 80501 | 18:1802242 |
| 80511 | 18:2001978 | 80533 | 18:2557098 | 80550 | 18:2832635 | 80557 | 18:2935768 | 80565 | 18:3102978 |
| 80575 | 18:3297749 | 80603 | 18:4036722 | 80612 | 18:4219952 | 80613 | 18:4226405 | 80620 | 18:4439946 |
| 80628 | 18:4565913 | 80644 | 18:4871817 | 80669 | 18:5415676 | 80670 | 18:5415711 | 80671 | 18:5416566 |
| 80712 | 18:6220320 | 80723 | 18:6398640 | 80725 | 18:6407051 | 80726 | 18:6407842 | 80727 | 18:6407856 |
| 80728 | 18:6408160 | 80729 | 18:6409137 | 80730 | 18:6409528 | 80748 | 18:6759745 | 80753 | 18:6859408 |
| 80770 | 18:7273399 | 80793 | 18:7769343 | 80802 | 18:7953745 | 80851 | 18:8931926 | 80872 | 18:9156689 |
| 80884 | 18:9413940 | 80885 | 18:9414216 | 80909 | 18:9827683 | 80924 | 18:10096838 | 80935 | 18:10422385 |
| 80980 | 18:11195149 | 81051 | 18:13077929 | 81083 | 18:13931354 | 81085 | 18:13962063 | 81086 | 18:13962211 |
| 81094 | 18:14131401 | 81128 | 18:15337353 | 81175 | 18:16420426 | 81195 | 18:16937183 | 81202 | 18:17081163 |
| 81213 | 18:17191494 | 81240 | 18:17819065 | 81249 | 18:18041884 | 81257 | 18:18196329 | 81262 | 18:18274003 |
| 81263 | 18:18274911 | 81264 | 18:18279929 | 81265 | 18:18284551 | 81266 | 18:18285334 | 81268 | 18:18287100 |
| 81270 | 18:18297472 | 81271 | 18:18300144 | 81289 | 18:18654230 | 81301 | 18:18883470 | 81336 | 18:19805409 |
| 81365 | 18:20391312 | 81369 | 18:20532028 | 81370 | 18:20581067 | 81371 | 18:20586213 | 81372 | 18:20591204 |
| 81373 | 18:20594221 | 81383 | 18:20851094 | 81434 | 18:22045866 | 81499 | 18:23267678 | 81519 | 18:23730169 |
| 81533 | 18:24068173 | 81584 | 18:25002158 | 81600 | 18:25402965 | 81616 | 18:25722314 | 81657 | 18:26620395 |
| 81660 | 18:26643719 | 81667 | 18:26781973 | 81699 | 18:27670793 | 81722 | 18:28212436 | 81754 | 18:29332445 |
| 81762 | 18:29394581 | 81775 | 18:29615496 | 81779 | 18:29751573 | 81780 | 18:29770581 | 81825 | 18:31291350 |
| 81850 | 18:31971311 | 81863 | 18:32267474 | 81869 | 18:32301839 | 81874 | 18:32426539 | 81917 | 18:33371652 |
| 81918 | 18:33381630 | 81919 | 18:33381846 | 81921 | 18:33389064 | 81930 | 18:33546416 | 81931 | 18:33559158 |
| 81956 | 18:34226859 | 82000 | 18:35324050 | 82017 | 18:35722943 | 82090 | 18:37440845 | 82096 | 18:37511943 |
| 82111 | 18:37857222 | 82117 | 18:37902748 | 82162 | 18:38995251 | 82167 | 18:39072678 | 82201 | 18:39856656 |
| 82211 | 18:40078593 | 82214 | 18:40135404 | 82268 | 18:41177791 | 82300 | 18:41952487 | 82332 | 18:42484052 |
| 82374 | 18:43224250 | 82381 | 18:43359712 | 82384 | 18:43378123 | 82402 | 18:43762058 | 82434 | 18:44688799 |
| 82436 | 18:44703246 | 82492 | 18:46177257 | 82495 | 18:46269611 | 82566 | 18:46759650 | 82608 | 18:47374533 |
| 82675 | 18:48506540 | 82731 | 18:49938198 | 82779 | 18:51259647 | 82786 | 18:51451371 | 82787 | 18:51452185 |
| 82788 | 18:51455635 | 82792 | 18:51522776 | 82795 | 18:51579528 | 82817 | 18:51820303 | 82847 | 18:52482506 |
| 82860 | 18:52950856 | 82873 | 18:53350033 | 82882 | 18:53554813 | 82896 | 18:53718739 | 82916 | 18:54039068 |
| 82934 | 18:54414979 | 82973 | 18:55042223 | 83009 | 18:55891884 | 83050 | 18:56619470 | 83056 | 18:56702388 |
| 83061 | 18:56777892 | 83063 | 18:56780574 | 83064 | 18:56781239 | 83065 | 18:56788137 | 83066 | 18:56788537 |
| 83067 | 18:56790131 | 83068 | 18:56793472 | 83069 | 18:56793653 | 83070 | 18:56802310 | 83071 | 18:56802366 |
| 83072 | 18:56802408 | 83073 | 18:56803155 | 83074 | 18:56805465 | 83075 | 18:56805669 | 83076 | 18:56805763 |
| 83077 | 18:56805884 | 83078 | 18:56805908 | 83079 | 18:56805969 | 83080 | 18:56806452 | 83081 | 18:56806522 |
| 83082 | 18:56806817 | 83083 | 18:56807069 | 83084 | 18:56807143 | 83085 | 18:56807200 | 83086 | 18:56808520 |
| 83087 | 18:56808693 | 83088 | 18:56808739 | 83089 | 18:56809175 | 83090 | 18:56809229 | 83091 | 18:56809274 |
| 83092 | 18:56809314 | 83093 | 18:56809399 | 83094 | 18:56811400 | 83095 | 18:56816836 | 83096 | 18:56817529 |
| 83097 | 18:56817530 | 83098 | 18:56817556 | 83099 | 18:56817983 | 83100 | 18:56819032 | 83102 | 18:56819579 |
| 83103 | 18:56820099 | 83104 | 18:56820288 | 83105 | 18:56820485 | 83106 | 18:56821287 | 83129 | 18:57480828 |
| 83131 | 18:57489304 | 83132 | 18:57496016 | 83135 | 18:57545249 | 83136 | 18:57549761 | 83137 | 18:57559274 |
| 83154 | 18:57826436 | 83155 | 18:57826437 | 83164 | 18:57929781 | 83230 | 18:58590500 | 83236 | 18:58874702 |
| 83262 | 18:60020922 | 83277 | 18:60611535 | 83278 | 18:60661061 | 83292 | 18:61082028 | 83295 | 18:61224849 |
| 83317 | 18:61691526 | 83377 | 18:62817960 | 83416 | 18:63491911 | 83425 | 18:63743739 | 83431 | 18:63828080 |
| 83432 | 18:63835095 | 83437 | 18:63865767 | 83560 | 18:65350149 | 83571 | 18:65487247 | 83589 | 19:153194 |
| 83605 | 19:905203 | 83670 | 19:3029129 | 83693 | 19:3751571 | 83731 | 19:4866448 | 83744 | 19:5165734 |
| 83761 | 19:5576963 | 83775 | 19:5960251 | 83792 | 19:6497947 | 83798 | 19:6642039 | 83815 | 19:6924319 |
| 83883 | 19:8123606 | 83898 | 19:8310681 | 83901 | 19:8332089 | 83902 | 19:8333798 | 83903 | 19:8344332 |
| 83904 | 19:8344354 | 83905 | 19:8356008 | 83906 | 19:8366630 | 83907 | 19:8366749 | 83916 | 19:8476307 |
| 83918 | 19:8521460 | 83930 | 19:8822288 | 83958 | 19:9228505 | 83982 | 19:9886007 | 83992 | 19:10035802 |
| 83998 | 19:10157460 | 83999 | 19:10157552 | 84100 | 19:12546324 | 84101 | 19:12551345 | 84104 | 19:12587276 |
| 84139 | 19:13390721 | 84143 | 19:13523234 | 84144 | 19:13523312 | 84145 | 19:13523394 | 84146 | 19:13523599 |
| 84147 | 19:13523669 | 84148 | 19:13523920 | 84149 | 19:13524217 | 84150 | 19:13524534 | 84151 | 19:13524690 |
| 84152 | 19:13525738 | 84191 | 19:14079144 | 84213 | 19:14542746 | 84214 | 19:14547882 | 84228 | 19:14975147 |
| 84229 | 19:14976930 | 84230 | 19:14977190 | 84240 | 19:15153935 | 84243 | 19:15176665 | 84277 | 19:15979571 |
| 84333 | 19:16959557 | 84403 | 19:18344112 | 84406 | 19:18364792 | 84491 | 19:20068707 | 84575 | 19:21354124 |
| 84635 | 19:22407739 | 84683 | 19:23510771 | 84701 | 19:23901688 | 84718 | 19:24121007 | 84738 | 19:24347182 |
| 84800 | 19:25069906 | 84803 | 19:25117527 | 84804 | 19:25118864 | 84812 | 19:25238500 | 84840 | 19:25573317 |
| 84841 | 19:25583861 | 84842 | 19:25635561 | 84880 | 19:26078995 | 84883 | 19:26146723 | 84884 | 19:26158447 |
| 84885 | 19:26162940 | 84886 | 19:26166792 | 84887 | 19:26177724 | 84888 | 19:26180605 | 84899 | 19:26370592 |
| 84901 | 19:26384973 | 84902 | 19:26386173 | 84904 | 19:26386998 | 84905 | 19:26408649 | 84906 | 19:26414194 |
| 84907 | 19:26425088 | 84908 | 19:26428880 | 84910 | 19:26432937 | 84912 | 19:26434534 | 84916 | 19:26440887 |
| 84917 | 19:26442377 | 84918 | 19:26442417 | 84922 | 19:26458156 | 84925 | 19:26462109 | 84928 | 19:26466738 |
| 84930 | 19:26468564 | 84931 | 19:26485885 | 84932 | 19:26489724 | 84936 | 19:26508139 | 84939 | 19:26510614 |
| 84942 | 19:26555053 | 84944 | 19:26575215 | 84945 | 19:26588232 | 84946 | 19:26595700 | 84947 | 19:26595755 |
| 84948 | 19:26599010 | 84950 | 19:26628925 | 84951 | 19:26662321 | 84953 | 19:26675470 | 84954 | 19:26676204 |
| 84956 | 19:26697210 | 84958 | 19:26705636 | 84959 | 19:26707790 | 84960 | 19:26719756 | 84961 | 19:26732472 |
| 84963 | 19:26744245 | 85040 | 19:28793496 | 85083 | 19:29602130 | 85084 | 19:29603381 | 85102 | 19:29967023 |
| 85123 | 19:30488002 | 85130 | 19:30639326 | 85131 | 19:30644348 | 85132 | 19:30645410 | 85133 | 19:30648555 |
| 85134 | 19:30696412 | 85137 | 19:30818025 | 85139 | 19:30826714 | 85167 | 19:31463142 | 85168 | 19:31485672 |
| 85183 | 19:31907703 | 85187 | 19:31968664 | 85207 | 19:32399697 | 85219 | 19:32665116 | 85236 | 19:33046409 |
| 85285 | 19:34025535 | 85395 | 19:35866723 | 85400 | 19:35970366 | 85434 | 19:36329939 | 85487 | 19:37333280 |
| 85581 | 19:40048911 | 85595 | 19:40251758 | 85597 | 19:40279227 | 85615 | 19:40640928 | 85635 | 19:40995967 |
| 85654 | 19:41486757 | 85668 | 19:41704492 | 85669 | 19:41719174 | 85686 | 19:41943823 | 85702 | 19:42161619 |
| 85726 | 19:42580737 | 85734 | 19:42764834 | 85760 | 19:43424335 | 85761 | 19:43424535 | 85762 | 19:43428799 |
| 85763 | 19:43431509 | 85764 | 19:43433116 | 85765 | 19:43435751 | 85766 | 19:43438458 | 85770 | 19:43493558 |
| 85778 | 19:43722064 | 85864 | 19:45668089 | 85868 | 19:45717594 | 85869 | 19:45717924 | 85870 | 19:45717976 |
| 85871 | 19:45717979 | 85872 | 19:45718107 | 85873 | 19:45718307 | 85874 | 19:45718340 | 85875 | 19:45720202 |
| 85876 | 19:45720264 | 85877 | 19:45720488 | 85878 | 19:45720862 | 85879 | 19:45736048 | 85880 | 19:45738664 |
| 85881 | 19:45740485 | 85882 | 19:45741144 | 85883 | 19:45741389 | 85884 | 19:45741952 | 85885 | 19:45742152 |
| 85886 | 19:45742153 | 85894 | 19:45834449 | 85995 | 19:48080018 | 86018 | 19:48469710 | 86020 | 19:48505288 |
| 86052 | 19:49235212 | 86059 | 19:49331336 | 86060 | 19:49332593 | 86117 | 19:50655487 | 86124 | 19:50694652 |
| 86125 | 19:50699155 | 86126 | 19:50703436 | 86127 | 19:50711021 | 86128 | 19:50712302 | 86129 | 19:50714204 |
| 86130 | 19:50715174 | 86131 | 19:50716477 | 86132 | 19:50718075 | 86133 | 19:50724757 | 86134 | 19:50732339 |
| 86135 | 19:50734637 | 86136 | 19:50741020 | 86137 | 19:50772016 | 86210 | 19:51559279 | 86211 | 19:51559359 |
| 86239 | 19:52037026 | 86255 | 19:52551695 | 86317 | 19:53746270 | 86327 | 19:53883618 | 86328 | 19:53883934 |
| 86336 | 19:54027650 | 86340 | 19:54047174 | 86359 | 19:54403366 | 86370 | 19:54621153 | 86373 | 19:54664915 |
| 86374 | 19:54665643 | 86375 | 19:54667304 | 86377 | 19:54672612 | 86392 | 19:54949422 | 86397 | 19:55036335 |
| 86412 | 19:55420287 | 86545 | 19:56454067 | 86547 | 19:56460077 | 86568 | 19:56726108 | 86584 | 19:57037210 |
| 86601 | 19:57419304 | 86605 | 19:57490381 | 86606 | 19:57508171 | 86615 | 19:57670291 | 86624 | 19:57908956 |
| 86637 | 19:58189519 | 86654 | 19:58502690 | 86661 | 19:58677753 | 86662 | 19:58689620 | 86691 | 19:59301702 |
| 86694 | 19:59353477 | 86714 | 19:59622681 | 86730 | 19:59843660 | 86735 | 19:59905716 | 86755 | 19:60165330 |
| 86757 | 19:60175972 | 86758 | 19:60176459 | 86760 | 19:60192077 | 86784 | 19:60532635 | 86793 | 19:60643681 |
| 86797 | 19:60699603 | 86803 | 19:60819634 | 86816 | 19:61121915 | 86853 | 19:61536597 | 86863 | 19:61642528 |
| 86864 | 19:61642559 | 86875 | 19:61831896 | 86896 | 19:62236608 | 86901 | 19:62290414 | 86906 | 19:62421264 |
| 86921 | 19:62751964 | 86923 | 19:62757779 | 86937 | 19:62914703 | 86971 | 20:465779 | 86984 | 20:581428 |
| 86985 | 20:583434 | 87026 | 20:1587207 | 87034 | 20:1637477 | 87046 | 20:1840861 | 87049 | 20:1872065 |
| 87081 | 20:2302262 | 87098 | 20:2623153 | 87109 | 20:3049545 | 87110 | 20:3050301 | 87111 | 20:3052518 |
| 87112 | 20:3056100 | 87113 | 20:3056394 | 87114 | 20:3061100 | 87115 | 20:3061457 | 87116 | 20:3062741 |
| 87117 | 20:3064116 | 87118 | 20:3064279 | 87119 | 20:3065002 | 87120 | 20:3065571 | 87121 | 20:3065997 |
| 87185 | 20:4419534 | 87443 | 20:5918697 | 87517 | 20:7068652 | 87549 | 20:7706966 | 87560 | 20:7984208 |
| 87598 | 20:8931603 | 87671 | 20:10580364 | 87672 | 20:10581845 | 87697 | 20:11193697 | 87724 | 20:11666353 |
| 87731 | 20:11849546 | 87737 | 20:12016234 | 87780 | 20:12743838 | 87791 | 20:13014120 | 87797 | 20:13145133 |
| 87811 | 20:13530219 | 87817 | 20:13767878 | 87836 | 20:14332360 | 87852 | 20:15273040 | 87864 | 20:15564869 |
| 87875 | 20:15869027 | 87906 | 20:16621914 | 87909 | 20:16650192 | 87961 | 20:18107968 | 87991 | 20:19026521 |
| 87993 | 20:19083370 | 88011 | 20:19606439 | 88019 | 20:19755587 | 88078 | 20:21474908 | 88083 | 20:21584291 |
| 88117 | 20:22063416 | 88191 | 20:22598687 | 88200 | 20:22757553 | 88209 | 20:22931278 | 88259 | 20:23864507 |
| 88277 | 20:23991980 | 88323 | 20:25147490 | 88359 | 20:26491689 | 88360 | 20:26496401 | 88361 | 20:26502059 |
| 88362 | 20:26504123 | 88363 | 20:26514971 | 88364 | 20:26518907 | 88366 | 20:26522200 | 88406 | 20:27651444 |
| 88407 | 20:27665296 | 88420 | 20:27980203 | 88434 | 20:28277863 | 88467 | 20:28789560 | 88482 | 20:29236907 |
| 88526 | 20:30411387 | 88535 | 20:30681003 | 88568 | 20:31716095 | 88572 | 20:31786758 | 88574 | 20:31871902 |
| 88685 | 20:34576290 | 88806 | 20:37374849 | 88817 | 20:37489422 | 88837 | 20:37861824 | 88857 | 20:38404986 |
| 88885 | 20:39228116 | 88889 | 20:39268509 | 88899 | 20:39457698 | 88902 | 20:39467997 | 88907 | 20:39535650 |
| 88913 | 20:39565695 | 88931 | 20:39982244 | 88942 | 20:40219777 | 88959 | 20:40746359 | 88976 | 20:41050966 |
| 89002 | 20:41628336 | 89045 | 20:42462021 | 89117 | 20:44485586 | 89140 | 20:45144462 | 89147 | 20:45457044 |
| 89148 | 20:45547831 | 89150 | 20:45567543 | 89154 | 20:45642750 | 89156 | 20:45675568 | 89158 | 20:45694391 |
| 89174 | 20:46029522 | 89175 | 20:46040337 | 89176 | 20:46040344 | 89177 | 20:46043226 | 89189 | 20:46349920 |
| 89197 | 20:46707839 | 89206 | 20:47136685 | 89214 | 20:47190891 | 89228 | 20:47736047 | 89277 | 20:49126008 |
| 89278 | 20:49182818 | 89301 | 20:49941637 | 89316 | 20:50311371 | 89348 | 20:51482831 | 89384 | 20:52684364 |
| 89407 | 20:53375334 | 89412 | 20:53445710 | 89417 | 20:53542795 | 89468 | 20:55149259 | 89470 | 20:55168267 |
| 89471 | 20:55169154 | 89474 | 20:55188763 | 89476 | 20:55206284 | 89477 | 20:55207791 | 89481 | 20:55321910 |
| 89488 | 20:55507243 | 89626 | 20:58393406 | 89647 | 20:58770472 | 89651 | 20:58809259 | 89694 | 20:59879577 |
| 89697 | 20:59895328 | 89741 | 20:61269332 | 89769 | 20:61983565 | 89777 | 20:62202768 | 89791 | 20:62523219 |
| 89834 | 20:63451088 | 89878 | 20:64194501 | 89901 | 20:64754306 | 89915 | 20:65165202 | 89925 | 20:65480230 |
| 89931 | 20:65608403 | 89992 | 20:66984199 | 90045 | 20:68049361 | 90056 | 20:68247302 | 90060 | 20:68312623 |
| 90065 | 20:68359426 | 90126 | 20:69498583 | 90156 | 20:70136618 | 90175 | 20:70711203 | 90290 | 21:560149 |
| 90307 | 21:1776376 | 90351 | 21:3330534 | 90366 | 21:3866539 | 90394 | 21:4290924 | 90412 | 21:4894437 |
| 90448 | 21:5848777 | 90485 | 21:7043405 | 90502 | 21:7479548 | 90518 | 21:7765157 | 90533 | 21:8066355 |
| 90536 | 21:8077420 | 90537 | 21:8084411 | 90539 | 21:8091687 | 90540 | 21:8093756 | 90541 | 21:8096108 |
| 90544 | 21:8098084 | 90545 | 21:8100095 | 90547 | 21:8121506 | 90548 | 21:8123911 | 90550 | 21:8132548 |
| 90564 | 21:8374844 | 90580 | 21:8819472 | 90593 | 21:9141650 | 90611 | 21:9612990 | 90614 | 21:9635812 |
| 90615 | 21:9642385 | 90658 | 21:10706242 | 90672 | 21:10964762 | 90688 | 21:11397271 | 90715 | 21:12027109 |
| 90738 | 21:12572473 | 90764 | 21:13059367 | 90801 | 21:13974032 | 90810 | 21:14159859 | 90836 | 21:14800811 |
| 90842 | 21:14976378 | 90881 | 21:15763479 | 90898 | 21:16325852 | 90899 | 21:16327666 | 90900 | 21:16352286 |
| 90902 | 21:16380714 | 90904 | 21:16401319 | 90907 | 21:16447288 | 90937 | 21:16900535 | 90938 | 21:16964578 |
| 90960 | 21:17491219 | 90980 | 21:18041395 | 91095 | 21:19926364 | 91108 | 21:20273386 | 91145 | 21:20915737 |
| 91166 | 21:21373080 | 91192 | 21:21922090 | 91199 | 21:22093113 | 91212 | 21:22196208 | 91297 | 21:24454479 |
| 91302 | 21:24572190 | 91312 | 21:24911518 | 91403 | 21:27617328 | 91405 | 21:27709157 | 91450 | 21:29090583 |
| 91467 | 21:29574040 | 91468 | 21:29585741 | 91469 | 21:29663335 | 91474 | 21:29793976 | 91529 | 21:31234804 |
| 91619 | 21:33320463 | 91621 | 21:33351272 | 91625 | 21:33376637 | 91660 | 21:33757910 | 91681 | 21:34100944 |
| 91710 | 21:34503640 | 91721 | 21:34672141 | 91744 | 21:35184835 | 91759 | 21:35593323 | 91773 | 21:35773499 |
| 91788 | 21:36015961 | 91795 | 21:36164785 | 91824 | 21:36934454 | 91828 | 21:37113932 | 91829 | 21:37118207 |
| 91830 | 21:37118300 | 91917 | 21:39931787 | 91933 | 21:40317765 | 91942 | 21:40547674 | 91996 | 21:41966823 |
| 92040 | 21:42868063 | 92065 | 21:43541273 | 92066 | 21:43549420 | 92077 | 21:43767846 | 92081 | 21:43869821 |
| 92105 | 21:44394987 | 92129 | 21:44779372 | 92150 | 21:45470174 | 92187 | 21:46513102 | 92213 | 21:47280539 |
| 92214 | 21:47282065 | 92215 | 21:47287202 | 92216 | 21:47288654 | 92217 | 21:47288689 | 92218 | 21:47289181 |
| 92220 | 21:47294064 | 92222 | 21:47296827 | 92223 | 21:47297692 | 92224 | 21:47297903 | 92225 | 21:47298236 |
| 92226 | 21:47298622 | 92227 | 21:47298661 | 92228 | 21:47298894 | 92229 | 21:47298942 | 92230 | 21:47299468 |
| 92231 | 21:47299536 | 92232 | 21:47299742 | 92233 | 21:47299950 | 92234 | 21:47300166 | 92236 | 21:47301069 |
| 92237 | 21:47301092 | 92238 | 21:47301705 | 92239 | 21:47301914 | 92240 | 21:47301979 | 92241 | 21:47302160 |
| 92242 | 21:47302405 | 92243 | 21:47302771 | 92244 | 21:47302793 | 92245 | 21:47303150 | 92246 | 21:47303326 |
| 92247 | 21:47303615 | 92248 | 21:47304710 | 92249 | 21:47308349 | 92250 | 21:47309250 | 92251 | 21:47314809 |
| 92253 | 21:47316827 | 92263 | 21:47572280 | 92286 | 21:48277772 | 92318 | 21:49185947 | 92319 | 21:49189566 |
| 92320 | 21:49193705 | 92321 | 21:49194179 | 92323 | 21:49195976 | 92324 | 21:49209645 | 92326 | 21:49218212 |
| 92327 | 21:49229215 | 92328 | 21:49231400 | 92360 | 21:50042557 | 92372 | 21:50467495 | 92407 | 21:51606765 |
| 92424 | 21:52027284 | 92433 | 21:52289877 | 92446 | 21:52553332 | 92471 | 21:53340020 | 92497 | 21:54060629 |
| 92505 | 21:54338413 | 92522 | 21:54761061 | 92523 | 21:54761327 | 92524 | 21:54766467 | 92526 | 21:54773661 |
| 92527 | 21:54779429 | 92528 | 21:54795770 | 92570 | 21:55568962 | 92571 | 21:55571357 | 92575 | 21:55603036 |
| 92577 | 21:55606404 | 92645 | 21:56889237 | 92646 | 21:56907066 | 92724 | 21:57243795 | 92757 | 21:58266418 |
| 92773 | 21:58557250 | 92819 | 21:59483269 | 92840 | 21:59828548 | 92841 | 21:59838076 | 92844 | 21:59872593 |
| 92852 | 21:59962135 | 92853 | 21:59965050 | 92861 | 21:60071151 | 92876 | 21:60290443 | 92897 | 21:60717146 |
| 92945 | 21:61871337 | 92978 | 21:62509823 | 92981 | 21:62565907 | 92997 | 21:62953263 | 93021 | 21:63719399 |
| 93022 | 21:63775744 | 93035 | 21:64020970 | 93043 | 21:64322263 | 93045 | 21:64323466 | 93054 | 21:64458851 |
| 93055 | 21:64468813 | 93061 | 21:64551281 | 93067 | 21:64674944 | 93071 | 21:64702159 | 93075 | 21:64763778 |
| 93111 | 21:65219263 | 93113 | 21:65222945 | 93120 | 21:65350726 | 93122 | 21:65389454 | 93124 | 21:65436920 |
| 93127 | 21:65495121 | 93138 | 21:65745850 | 93203 | 21:67086290 | 93205 | 21:67097791 | 93207 | 21:67137594 |
| 93216 | 21:67459394 | 93217 | 21:67461089 | 93236 | 21:67933900 | 93237 | 21:67933901 | 93267 | 21:68505684 |
| 93284 | 21:68792488 | 93291 | 21:68917772 | 93302 | 21:69177495 | 93307 | 21:69279748 | 93384 | 22:1336082 |
| 93421 | 22:2092658 | 93448 | 22:2744263 | 93451 | 22:2759833 | 93452 | 22:2768731 | 93476 | 22:3271155 |
| 93490 | 22:3549590 | 93492 | 22:3565222 | 93512 | 22:4182249 | 93517 | 22:4255887 | 93535 | 22:4575207 |
| 93596 | 22:5655551 | 93689 | 22:7518330 | 93690 | 22:7526334 | 93700 | 22:7714187 | 93721 | 22:8392537 |
| 93725 | 22:8430070 | 93741 | 22:9021813 | 93749 | 22:9259333 | 93782 | 22:9998563 | 93798 | 22:10114240 |
| 93824 | 22:10733620 | 93845 | 22:11198997 | 93857 | 22:11572590 | 93875 | 22:11958308 | 93895 | 22:12274925 |
| 93927 | 22:12850291 | 93957 | 22:13771925 | 93999 | 22:14949092 | 94000 | 22:14961326 | 94003 | 22:15026827 |
| 94021 | 22:15501897 | 94034 | 22:15667257 | 94037 | 22:15711517 | 94056 | 22:16388677 | 94061 | 22:16514054 |
| 94079 | 22:16739297 | 94121 | 22:17893434 | 94128 | 22:18003077 | 94129 | 22:18033855 | 94139 | 22:18259710 |
| 94140 | 22:18265709 | 94154 | 22:18559489 | 94166 | 22:18849789 | 94167 | 22:18851178 | 94178 | 22:19237876 |
| 94184 | 22:19372836 | 94197 | 22:19837982 | 94198 | 22:19847979 | 94213 | 22:20176939 | 94229 | 22:20643619 |
| 94230 | 22:20646235 | 94241 | 22:20897046 | 94278 | 22:21648235 | 94280 | 22:21664858 | 94296 | 22:21953904 |
| 94303 | 22:22190483 | 94309 | 22:22421295 | 94314 | 22:22604015 | 94324 | 22:22885054 | 94352 | 22:23611781 |
| 94362 | 22:23913434 | 94413 | 22:25649715 | 94420 | 22:25917253 | 94440 | 22:26167092 | 94456 | 22:26660485 |
| 94489 | 22:27621315 | 94505 | 22:28117808 | 94510 | 22:28166101 | 94521 | 22:28401484 | 94530 | 22:28491057 |
| 94568 | 22:29357579 | 94579 | 22:29669451 | 94592 | 22:29900170 | 94598 | 22:30126912 | 94643 | 22:31208133 |
| 94672 | 22:32149285 | 94703 | 22:33059262 | 94715 | 22:33350762 | 94739 | 22:34116071 | 94741 | 22:34136171 |
| 94756 | 22:34692656 | 94757 | 22:34695203 | 94773 | 22:35027110 | 94777 | 22:35094672 | 94779 | 22:35111999 |
| 94789 | 22:35301748 | 94814 | 22:35855583 | 94827 | 22:36060507 | 94829 | 22:36134170 | 94835 | 22:36318773 |
| 94861 | 22:37054760 | 94868 | 22:37235828 | 94896 | 22:37669398 | 94913 | 22:38264582 | 94914 | 22:38283974 |
| 94923 | 22:38466243 | 94937 | 22:38695645 | 94941 | 22:38735989 | 94942 | 22:38740955 | 94960 | 22:39188813 |
| 94989 | 22:39567152 | 94995 | 22:39656245 | 94997 | 22:39682658 | 95055 | 22:41016011 | 95066 | 22:41417903 |
| 95081 | 22:41777356 | 95084 | 22:41792218 | 95085 | 22:41797029 | 95089 | 22:41877932 | 95149 | 22:43286201 |
| 95155 | 22:43386150 | 95178 | 22:44035160 | 95210 | 22:44640940 | 95237 | 22:45552005 | 95247 | 22:45691255 |
| 95263 | 22:45913562 | 95274 | 22:46107748 | 95276 | 22:46144763 | 95297 | 22:46746909 | 95317 | 22:47040045 |
| 95323 | 22:47111303 | 95426 | 22:49820256 | 95433 | 22:49940593 | 95434 | 22:49970190 | 95440 | 22:50067364 |
| 95499 | 22:51502260 | 95500 | 22:51503045 | 95531 | 22:52333167 | 95540 | 22:52556549 | 95558 | 22:52884674 |
| 95565 | 22:52961463 | 95592 | 22:53497101 | 95621 | 22:54011562 | 95672 | 22:55011602 | 95677 | 22:55091224 |
| 95695 | 22:55323748 | 95712 | 22:55563765 | 95758 | 22:56293581 | 95793 | 22:56540115 | 95794 | 22:56540668 |
| 95795 | 22:56542873 | 95796 | 22:56544054 | 95798 | 22:56573939 | 95850 | 22:57521486 | 95882 | 22:57942911 |
| 95894 | 22:58136676 | 95910 | 22:58494061 | 95920 | 22:58678398 | 95922 | 22:58700947 | 95924 | 22:58773917 |
| 95941 | 22:59128229 | 95967 | 22:59629485 | 95973 | 22:59712330 | 95985 | 22:59846886 | 96044 | 23:774985 |
| 96050 | 23:968798 | 96057 | 23:1194189 | 96063 | 23:1325388 | 96069 | 23:1434392 | 96095 | 23:2175010 |
| 96116 | 23:2637687 | 96164 | 23:3563217 | 96210 | 23:4375629 | 96230 | 23:4759481 | 96241 | 23:4872845 |
| 96246 | 23:5028069 | 96328 | 23:6757709 | 96342 | 23:7060820 | 96378 | 23:7879887 | 96380 | 23:7903756 |
| 96398 | 23:8328018 | 96449 | 23:9180909 | 96470 | 23:9852522 | 96499 | 23:10263390 | 96518 | 23:10703356 |
| 96522 | 23:10786602 | 96547 | 23:11354447 | 96568 | 23:11793160 | 96578 | 23:12035181 | 96579 | 23:12038531 |
| 96580 | 23:12039512 | 96582 | 23:12047337 | 96621 | 23:12878298 | 96665 | 23:13769527 | 96690 | 23:14024594 |
| 96691 | 23:14041498 | 96693 | 23:14067842 | 96705 | 23:14437275 | 96707 | 23:14454128 | 96722 | 23:14695783 |
| 96724 | 23:14770597 | 96737 | 23:14938161 | 96742 | 23:15032946 | 96784 | 23:15623072 | 96791 | 23:15668311 |
| 96800 | 23:15846192 | 96807 | 23:16055984 | 96852 | 23:17393860 | 96860 | 23:17578001 | 96864 | 23:17628981 |
| 96866 | 23:17657910 | 96905 | 23:18560341 | 96911 | 23:18766462 | 96916 | 23:18918643 | 96964 | 23:19690687 |
| 96967 | 23:19754433 | 96969 | 23:19762716 | 96978 | 23:19935139 | 96980 | 23:19938929 | 96995 | 23:20172115 |
| 97011 | 23:20449980 | 97015 | 23:20553981 | 97016 | 23:20554380 | 97017 | 23:20555345 | 97018 | 23:20555464 |
| 97019 | 23:20555512 | 97020 | 23:20555877 | 97046 | 23:21266809 | 97053 | 23:21371538 | 97074 | 23:22025892 |
| 97082 | 23:22189816 | 97084 | 23:22253261 | 97099 | 23:22609075 | 97100 | 23:22632358 | 97120 | 23:23494563 |
| 97121 | 23:23495169 | 97122 | 23:23515623 | 97128 | 23:23599173 | 97131 | 23:23663060 | 97133 | 23:23729292 |
| 97144 | 23:23841412 | 97166 | 23:24310745 | 97182 | 23:24584592 | 97220 | 23:24825942 | 97235 | 23:24996122 |
| 97236 | 23:25010458 | 97276 | 23:25343657 | 97294 | 23:25495218 | 97307 | 23:25727838 | 97371 | 23:27168873 |
| 97396 | 23:27404973 | 97400 | 23:27427125 | 97416 | 23:27534819 | 97418 | 23:27545798 | 97509 | 23:28274177 |
| 97515 | 23:28328479 | 97516 | 23:28329217 | 97524 | 23:28449487 | 97546 | 23:28783122 | 97592 | 23:29113119 |
| 97631 | 23:30057510 | 97657 | 23:30485309 | 97658 | 23:30487092 | 97701 | 23:31528534 | 97733 | 23:32121998 |
| 97741 | 23:32302519 | 97742 | 23:32303395 | 97743 | 23:32303732 | 97744 | 23:32304775 | 97745 | 23:32304794 |
| 97746 | 23:32305503 | 97748 | 23:32348823 | 97818 | 23:33768810 | 97821 | 23:33852032 | 97853 | 23:34945913 |
| 97854 | 23:34946818 | 97855 | 23:34946868 | 97856 | 23:34948119 | 97858 | 23:35009819 | 97867 | 23:35427374 |
| 97893 | 23:36297029 | 97899 | 23:36392895 | 97941 | 23:37537593 | 97944 | 23:37550813 | 97949 | 23:37653049 |
| 97985 | 23:38817188 | 97986 | 23:38818425 | 98075 | 23:39639975 | 98079 | 23:39681488 | 98094 | 23:39977867 |
| 98134 | 23:40742268 | 98190 | 23:41726170 | 98215 | 23:42235597 | 98220 | 23:42301353 | 98255 | 23:43110372 |
| 98280 | 23:43675243 | 98300 | 23:44151531 | 98315 | 23:44315474 | 98337 | 23:44789129 | 98433 | 23:46714094 |
| 98477 | 23:47504887 | 98478 | 23:47505469 | 98507 | 23:47898321 | 98510 | 23:47934746 | 98513 | 23:47974902 |
| 98545 | 23:48734960 | 98548 | 23:48756252 | 98551 | 23:48811144 | 98563 | 23:49008700 | 98572 | 23:49166634 |
| 98603 | 23:49594224 | 98626 | 23:49959432 | 98629 | 23:50008283 | 98643 | 23:50262003 | 98652 | 23:50392532 |
| 98685 | 23:51016737 | 98702 | 23:51334319 | 98749 | 23:52192597 | 98751 | 23:52225791 | 98752 | 23:52234534 |
| 98753 | 23:52235518 | 98768 | 24:90983 | 98820 | 24:1186935 | 98822 | 24:1209847 | 98833 | 24:1549261 |
| 98855 | 24:1914090 | 98878 | 24:2417031 | 98920 | 24:3326601 | 98976 | 24:4463662 | 98988 | 24:4937656 |
| 98990 | 24:4964946 | 98992 | 24:4997353 | 99007 | 24:5345133 | 99010 | 24:5453007 | 99011 | 24:5453574 |
| 99019 | 24:5665620 | 99110 | 24:8068578 | 99117 | 24:8174902 | 99126 | 24:8403392 | 99156 | 24:9223598 |
| 99164 | 24:9437183 | 99183 | 24:9782804 | 99185 | 24:9813237 | 99187 | 24:9859970 | 99197 | 24:10080656 |
| 99198 | 24:10082192 | 99199 | 24:10083376 | 99200 | 24:10084233 | 99201 | 24:10085368 | 99202 | 24:10085413 |
| 99203 | 24:10085426 | 99204 | 24:10085505 | 99205 | 24:10085865 | 99206 | 24:10086080 | 99207 | 24:10086129 |
| 99208 | 24:10086191 | 99209 | 24:10087002 | 99210 | 24:10087384 | 99211 | 24:10087677 | 99212 | 24:10088340 |
| 99213 | 24:10089300 | 99214 | 24:10090958 | 99215 | 24:10092604 | 99216 | 24:10092904 | 99217 | 24:10093339 |
| 99218 | 24:10093371 | 99219 | 24:10093377 | 99220 | 24:10093477 | 99221 | 24:10093552 | 99222 | 24:10093626 |
| 99223 | 24:10099234 | 99224 | 24:10100942 | 99225 | 24:10101971 | 99226 | 24:10102022 | 99227 | 24:10102315 |
| 99228 | 24:10102992 | 99229 | 24:10104790 | 99230 | 24:10106712 | 99231 | 24:10112256 | 99232 | 24:10120280 |
| 99233 | 24:10120318 | 99234 | 24:10125493 | 99235 | 24:10127888 | 99236 | 24:10128409 | 99237 | 24:10128428 |
| 99238 | 24:10128467 | 99239 | 24:10129710 | 99240 | 24:10129815 | 99241 | 24:10131022 | 99242 | 24:10131061 |
| 99243 | 24:10131076 | 99244 | 24:10131213 | 99245 | 24:10131371 | 99246 | 24:10131470 | 99247 | 24:10131562 |
| 99248 | 24:10132898 | 99249 | 24:10132907 | 99250 | 24:10134379 | 99251 | 24:10134487 | 99252 | 24:10134521 |
| 99253 | 24:10134705 | 99254 | 24:10134719 | 99255 | 24:10134821 | 99256 | 24:10134859 | 99257 | 24:10134952 |
| 99258 | 24:10134953 | 99259 | 24:10136095 | 99260 | 24:10137902 | 99261 | 24:10137983 | 99262 | 24:10139078 |
| 99263 | 24:10139760 | 99264 | 24:10140006 | 99265 | 24:10140938 | 99266 | 24:10143195 | 99267 | 24:10143925 |
| 99268 | 24:10144011 | 99269 | 24:10144116 | 99270 | 24:10150408 | 99271 | 24:10151986 | 99272 | 24:10153446 |
| 99273 | 24:10155422 | 99291 | 24:10548737 | 99303 | 24:10838109 | 99313 | 24:11081707 | 99347 | 24:11895230 |
| 99374 | 24:12693269 | 99378 | 24:12740073 | 99397 | 24:13255685 | 99401 | 24:13395143 | 99406 | 24:13474854 |
| 99411 | 24:13599253 | 99431 | 24:14202286 | 99432 | 24:14280523 | 99460 | 24:14990004 | 99467 | 24:15215610 |
| 99471 | 24:15361519 | 99472 | 24:15366916 | 99495 | 24:16259312 | 99498 | 24:16327453 | 99514 | 24:16832720 |
| 99517 | 24:16888113 | 99550 | 24:17884468 | 99553 | 24:17978572 | 99564 | 24:18322902 | 99588 | 24:18993346 |
| 99621 | 24:20085016 | 99710 | 24:21387050 | 99713 | 24:21396719 | 99714 | 24:21407863 | 99721 | 24:21479759 |
| 99823 | 24:24049399 | 99835 | 24:24317905 | 99862 | 24:24997696 | 99899 | 24:25782726 | 99918 | 24:26095475 |
| 99926 | 24:26225994 | 99966 | 24:27495986 | 100001 | 24:28406831 | 100007 | 24:28544002 | 100008 | 24:28549242 |
| 100020 | 24:28829549 | 100028 | 24:29046077 | 100072 | 24:30123139 | 100195 | 24:32865289 | 100241 | 24:34044145 |
| 100253 | 24:34324032 | 100282 | 24:35161423 | 100295 | 24:35363476 | 100318 | 24:35787989 | 100341 | 24:36327209 |
| 100343 | 24:36389875 | 100434 | 24:39349798 | 100479 | 24:40319050 | 100480 | 24:40319355 | 100481 | 24:40320188 |
| 100482 | 24:40320243 | 100483 | 24:40320244 | 100484 | 24:40320423 | 100485 | 24:40320519 | 100486 | 24:40320531 |
| 100487 | 24:40320604 | 100488 | 24:40320642 | 100489 | 24:40320662 | 100493 | 24:40346626 | 100494 | 24:40351905 |
| 100624 | 24:43050450 | 100628 | 24:43264177 | 100641 | 24:43757869 | 100648 | 24:44070040 | 100658 | 24:44312012 |
| 100666 | 24:44454841 | 100676 | 24:44630854 | 100686 | 24:44900221 | 100693 | 24:45024408 | 100739 | 24:45883707 |
| 100741 | 24:45892470 | 100742 | 24:45893302 | 100743 | 24:45900861 | 100744 | 24:45906465 | 100746 | 24:45915865 |
| 100747 | 24:45916571 | 100748 | 24:45917829 | 100751 | 24:45935654 | 100752 | 24:45946887 | 100754 | 24:45961034 |
| 100755 | 24:45965027 | 100756 | 24:45966438 | 100757 | 24:45966683 | 100759 | 24:46019888 | 100760 | 24:46021234 |
| 100761 | 24:46031614 | 100763 | 24:46039025 | 100764 | 24:46039978 | 100765 | 24:46040921 | 100766 | 24:46047288 |
| 100767 | 24:46050396 | 100768 | 24:46052145 | 100795 | 24:46519527 | 100909 | 24:49563537 | 101002 | 24:51861629 |
| 101004 | 24:51877952 | 101020 | 24:52249991 | 101021 | 24:52250875 | 101031 | 24:52380242 | 101037 | 24:52572272 |
| 101038 | 24:52579310 | 101084 | 24:53806202 | 101150 | 24:55068839 | 101215 | 24:56483606 | 101216 | 24:56487277 |
| 101238 | 24:56927299 | 101239 | 24:56931903 | 101240 | 24:56935898 | 101296 | 24:58059868 | 101301 | 24:58143848 |
| 101314 | 24:58393476 | 101323 | 24:58514430 | 101338 | 24:58871566 | 101384 | 24:59956065 | 101385 | 24:59956162 |
| 101410 | 24:60385381 | 101417 | 24:60520182 | 101451 | 24:61136358 | 101476 | 24:61660672 | 101516 | 25:326413 |
| 101558 | 25:1326500 | 101576 | 25:1480308 | 101586 | 25:1751313 | 101627 | 25:2262957 | 101628 | 25:2264339 |
| 101629 | 25:2264421 | 101630 | 25:2264518 | 101631 | 25:2264598 | 101632 | 25:2264608 | 101633 | 25:2264757 |
| 101634 | 25:2264786 | 101635 | 25:2265149 | 101636 | 25:2266070 | 101637 | 25:2266258 | 101638 | 25:2266451 |
| 101639 | 25:2266559 | 101640 | 25:2266588 | 101641 | 25:2266682 | 101642 | 25:2266930 | 101643 | 25:2267017 |
| 101644 | 25:2267496 | 101645 | 25:2267627 | 101646 | 25:2267877 | 101647 | 25:2267895 | 101648 | 25:2267998 |
| 101649 | 25:2267999 | 101650 | 25:2268074 | 101651 | 25:2268874 | 101652 | 25:2269160 | 101653 | 25:2269161 |
| 101654 | 25:2269296 | 101655 | 25:2269538 | 101656 | 25:2269574 | 101658 | 25:2324283 | 101682 | 25:2565490 |
| 101752 | 25:3236023 | 101754 | 25:3277932 | 101772 | 25:3531364 | 101773 | 25:3532276 | 101796 | 25:3846717 |
| 101813 | 25:4251363 | 101814 | 25:4273072 | 101822 | 25:4417956 | 101860 | 25:5178359 | 101869 | 25:5356785 |
| 101870 | 25:5358991 | 101873 | 25:5370164 | 101879 | 25:5487714 | 101881 | 25:5507570 | 101882 | 25:5518043 |
| 101883 | 25:5518349 | 101884 | 25:5518412 | 101885 | 25:5519000 | 101886 | 25:5519033 | 101887 | 25:5519088 |
| 101888 | 25:5519241 | 101889 | 25:5519377 | 101890 | 25:5520929 | 101891 | 25:5520954 | 101892 | 25:5523535 |
| 101893 | 25:5524957 | 101894 | 25:5527480 | 101895 | 25:5527963 | 101897 | 25:5563162 | 101898 | 25:5586659 |
| 101899 | 25:5591911 | 101900 | 25:5595291 | 101919 | 25:5986235 | 101925 | 25:6069855 | 101941 | 25:6361028 |
| 101974 | 25:7238527 | 101975 | 25:7238686 | 101976 | 25:7239004 | 102030 | 25:8519593 | 102038 | 25:8697220 |
| 102039 | 25:8699100 | 102040 | 25:8709323 | 102044 | 25:8764411 | 102045 | 25:8773984 | 102050 | 25:8963960 |
| 102062 | 25:9320778 | 102069 | 25:9426676 | 102098 | 25:10079477 | 102101 | 25:10133416 | 102130 | 25:10823773 |
| 102139 | 25:10973032 | 102156 | 25:11195704 | 102159 | 25:11212796 | 102197 | 25:12044897 | 102220 | 25:12552168 |
| 102233 | 25:12921926 | 102234 | 25:12926787 | 102247 | 25:13336640 | 102255 | 25:13422767 | 102260 | 25:13560729 |
| 102268 | 25:13702632 | 102278 | 25:14050888 | 102302 | 25:14538936 | 102313 | 25:14817768 | 102315 | 25:14858436 |
| 102321 | 25:14961033 | 102336 | 25:15333545 | 102366 | 25:15932671 | 102414 | 25:16848884 | 102430 | 25:17233999 |
| 102431 | 25:17237242 | 102453 | 25:17765386 | 102580 | 25:21144052 | 102582 | 25:21184609 | 102614 | 25:21797730 |
| 102625 | 25:22007781 | 102634 | 25:22296079 | 102638 | 25:22329885 | 102654 | 25:22669357 | 102664 | 25:22938890 |
| 102681 | 25:23411155 | 102683 | 25:23449496 | 102688 | 25:23538270 | 102704 | 25:23977366 | 102705 | 25:23979613 |
| 102718 | 25:24161354 | 102723 | 25:24271708 | 102744 | 25:24641425 | 102746 | 25:24667454 | 102812 | 25:25961592 |
| 102815 | 25:26009553 | 102839 | 25:26491942 | 102855 | 25:26753501 | 102879 | 25:27390789 | 102920 | 25:27781936 |
| 102995 | 25:29636939 | 103008 | 25:29838430 | 103009 | 25:29838448 | 103010 | 25:29839578 | 103011 | 25:29844263 |
| 103029 | 25:30146443 | 103069 | 25:31190020 | 103071 | 25:31229333 | 103077 | 25:31297977 | 103095 | 25:31607175 |
| 103137 | 25:32422851 | 103138 | 25:32423232 | 103144 | 25:32564396 | 103171 | 25:33134898 | 103180 | 25:33336358 |
| 103202 | 25:33763001 | 103215 | 25:34018085 | 103230 | 25:34358250 | 103264 | 25:34863162 | 103271 | 25:34993980 |
| 103316 | 25:35712925 | 103337 | 25:35998185 | 103345 | 25:36199960 | 103382 | 25:36479714 | 103458 | 25:37601488 |
| 103549 | 25:39461505 | 103552 | 25:39500543 | 103563 | 25:39824171 | 103564 | 25:39824495 | 103588 | 25:40196620 |
| 103614 | 25:40615618 | 103672 | 25:41358682 | 103730 | 26:1198222 | 103731 | 26:1198334 | 103736 | 26:1252329 |
| 103757 | 26:1854681 | 103763 | 26:2064954 | 103773 | 26:2329446 | 103779 | 26:2473264 | 103780 | 26:2489945 |
| 103783 | 26:2571028 | 103817 | 26:3140966 | 103839 | 26:3713174 | 103840 | 26:3717033 | 103890 | 26:4954983 |
| 103895 | 26:5073658 | 103908 | 26:5545287 | 103954 | 26:7011510 | 103955 | 26:7022453 | 103987 | 26:7885953 |
| 103994 | 26:8061770 | 103997 | 26:8088819 | 103998 | 26:8089138 | 104011 | 26:8426892 | 104013 | 26:8486858 |
| 104023 | 26:8674404 | 104044 | 26:9248331 | 104097 | 26:10405639 | 104108 | 26:10640932 | 104139 | 26:11551549 |
| 104181 | 26:12736512 | 104186 | 26:12824626 | 104187 | 26:12837527 | 104191 | 26:12927334 | 104200 | 26:13161640 |
| 104209 | 26:13230527 | 104210 | 26:13230553 | 104211 | 26:13230674 | 104212 | 26:13230708 | 104213 | 26:13230970 |
| 104222 | 26:13354058 | 104249 | 26:14098514 | 104289 | 26:15245070 | 104333 | 26:16547356 | 104338 | 26:16723176 |
| 104372 | 26:17376800 | 104391 | 26:17759010 | 104421 | 26:18446389 | 104448 | 26:18949002 | 104472 | 26:19421103 |
| 104482 | 26:19560868 | 104512 | 26:20617024 | 104518 | 26:20829028 | 104541 | 26:21273598 | 104546 | 26:21380205 |
| 104607 | 26:23188643 | 104638 | 26:24162535 | 104658 | 26:24594195 | 104703 | 26:25362443 | 104742 | 26:25786538 |
| 104760 | 26:26139139 | 104785 | 26:26718349 | 104786 | 26:26723790 | 104796 | 26:26900204 | 104798 | 26:26928555 |
| 104804 | 26:27118119 | 104805 | 26:27128850 | 104806 | 26:27130646 | 104838 | 26:27876991 | 104845 | 26:28054644 |
| 104848 | 26:28086960 | 104880 | 26:29151186 | 104896 | 26:29673193 | 104900 | 26:29804085 | 104964 | 26:31379196 |
| 105030 | 26:33062542 | 105092 | 26:34211364 | 105094 | 26:34259597 | 105106 | 26:34593375 | 105133 | 26:35102465 |
| 105148 | 26:35442752 | 105162 | 26:35826945 | 105182 | 26:36410448 | 105193 | 26:36629017 | 105247 | 26:38084778 |
| 105253 | 26:38264488 | 105256 | 26:38330297 | 105337 | 26:40288755 | 105419 | 26:42332291 | 105435 | 26:42682604 |
| 105459 | 26:43279886 | 105498 | 26:44209268 | 105541 | 26:45319249 | 105569 | 26:45612666 | 105579 | 26:45867614 |
| 105609 | 26:46499606 | 105625 | 26:46841840 | 105669 | 26:47841265 | 105671 | 26:47878623 | 105679 | 26:48135137 |
| 105685 | 26:48274013 | 105710 | 26:48791917 | 105723 | 26:49108450 | 105752 | 26:49581277 | 105772 | 26:50021533 |
| 105774 | 26:50046769 | 105777 | 26:50172060 | 105788 | 26:50483114 | 105810 | 26:50882810 | 105818 | 26:51112488 |
| 105830 | 26:51406962 | 105831 | 26:51407053 | 105834 | 26:51487956 | 105835 | 26:51520196 | 105917 | 27:1670150 |
| 105953 | 27:2495166 | 106018 | 27:4056109 | 106019 | 27:4058537 | 106020 | 27:4060363 | 106021 | 27:4060531 |
| 106022 | 27:4060533 | 106083 | 27:5431758 | 106169 | 27:7970865 | 106207 | 27:8914781 | 106212 | 27:8978523 |
| 106269 | 27:9933403 | 106272 | 27:9948916 | 106326 | 27:11708333 | 106333 | 27:11961138 | 106340 | 27:12169807 |
| 106364 | 27:12847147 | 106385 | 27:13313622 | 106392 | 27:13498868 | 106396 | 27:13539506 | 106400 | 27:13604776 |
| 106402 | 27:13623320 | 106407 | 27:13722354 | 106412 | 27:13774707 | 106419 | 27:13894721 | 106420 | 27:13894736 |
| 106421 | 27:13895111 | 106422 | 27:13895260 | 106423 | 27:13895824 | 106424 | 27:13895841 | 106425 | 27:13896100 |
| 106426 | 27:13896107 | 106427 | 27:13896340 | 106428 | 27:13896382 | 106429 | 27:13896475 | 106430 | 27:13896495 |
| 106431 | 27:13896544 | 106432 | 27:13896557 | 106433 | 27:13896621 | 106434 | 27:13896676 | 106435 | 27:13897078 |
| 106436 | 27:13897184 | 106437 | 27:13897225 | 106438 | 27:13898435 | 106439 | 27:13898864 | 106440 | 27:13899961 |
| 106441 | 27:13900283 | 106442 | 27:13900709 | 106443 | 27:13900927 | 106444 | 27:13900986 | 106445 | 27:13902739 |
| 106446 | 27:13903517 | 106447 | 27:13903525 | 106448 | 27:13903671 | 106449 | 27:13904229 | 106450 | 27:13904269 |
| 106470 | 27:14467858 | 106485 | 27:14760458 | 106488 | 27:14801389 | 106496 | 27:15058579 | 106515 | 27:15382547 |
| 106543 | 27:15918980 | 106546 | 27:16000987 | 106550 | 27:16074052 | 106581 | 27:16407816 | 106584 | 27:16464527 |
| 106621 | 27:17348090 | 106630 | 27:17555135 | 106696 | 27:19049000 | 106699 | 27:19103942 | 106702 | 27:19175688 |
| 106703 | 27:19178944 | 106719 | 27:19583156 | 106772 | 27:20734233 | 106813 | 27:21907032 | 106856 | 27:23015129 |
| 106857 | 27:23015519 | 106862 | 27:23162886 | 106912 | 27:24155175 | 106947 | 27:24823413 | 106953 | 27:24889870 |
| 106959 | 27:25111470 | 106983 | 27:25597037 | 106984 | 27:25632047 | 107016 | 27:26458109 | 107021 | 27:26491670 |
| 107054 | 27:27075217 | 107056 | 27:27089901 | 107083 | 27:27645675 | 107102 | 27:28160084 | 107117 | 27:28613312 |
| 107128 | 27:28845373 | 107136 | 27:29137179 | 107140 | 27:29174979 | 107174 | 27:29722415 | 107195 | 27:30336321 |
| 107197 | 27:30352172 | 107212 | 27:30751847 | 107214 | 27:30799722 | 107250 | 27:31405475 | 107260 | 27:31691203 |
| 107277 | 27:31936569 | 107278 | 27:31937059 | 107279 | 27:31937078 | 107280 | 27:31938645 | 107281 | 27:31939482 |
| 107282 | 27:31940571 | 107335 | 27:32941799 | 107337 | 27:32957490 | 107347 | 27:33063853 | 107381 | 27:34082682 |
| 107423 | 27:35043316 | 107460 | 27:35480823 | 107462 | 27:35485331 | 107463 | 27:35488554 | 107464 | 27:35489257 |
| 107467 | 27:35506982 | 107471 | 27:35556513 | 107473 | 27:35570251 | 107477 | 27:35709604 | 107485 | 27:35789498 |
| 107488 | 27:35863653 | 107493 | 27:35889925 | 107509 | 27:36291522 | 107516 | 27:36452785 | 107529 | 27:36735236 |
| 107551 | 27:37288845 | 107558 | 27:37353731 | 107561 | 27:37379099 | 107602 | 27:38144204 | 107631 | 27:38793276 |
| 107633 | 27:38844060 | 107660 | 27:39447790 | 107687 | 27:40218010 | 107781 | 27:42642792 | 107798 | 27:43019477 |
| 107802 | 27:43086918 | 107805 | 27:43182706 | 107818 | 27:43500535 | 107825 | 27:43689399 | 107856 | 27:44157299 |
| 107861 | 27:44229484 | 107863 | 27:44268404 | 107909 | 27:45312149 | 107946 | 28:1157605 | 107960 | 28:1342853 |
| 107998 | 28:2055151 | 108005 | 28:2304836 | 108028 | 28:3006891 | 108029 | 28:3047863 | 108030 | 28:3081340 |
| 108031 | 28:3085107 | 108075 | 28:5126492 | 108089 | 28:5287434 | 108099 | 28:5566930 | 108123 | 28:5966745 |
| 108195 | 28:7882083 | 108252 | 28:9096925 | 108271 | 28:9589074 | 108357 | 28:12204786 | 108358 | 28:12216204 |
| 108360 | 28:12230473 | 108361 | 28:12253261 | 108362 | 28:12259647 | 108363 | 28:12260824 | 108366 | 28:12274548 |
| 108367 | 28:12276973 | 108368 | 28:12280112 | 108369 | 28:12281177 | 108370 | 28:12285449 | 108371 | 28:12340669 |
| 108380 | 28:12499169 | 108397 | 28:12767375 | 108421 | 28:13530571 | 108425 | 28:13632810 | 108439 | 28:14061176 |
| 108459 | 28:14464098 | 108462 | 28:14523784 | 108537 | 28:16147418 | 108586 | 28:17255203 | 108587 | 28:17266697 |
| 108592 | 28:17297973 | 108594 | 28:17348454 | 108599 | 28:17452718 | 108600 | 28:17453580 | 108602 | 28:17454068 |
| 108603 | 28:17454138 | 108604 | 28:17454593 | 108605 | 28:17454620 | 108608 | 28:17536228 | 108612 | 28:17642513 |
| 108618 | 28:17917993 | 108673 | 28:19218143 | 108702 | 28:20070339 | 108726 | 28:20595474 | 108776 | 28:22093298 |
| 108823 | 28:23296337 | 108847 | 28:23859369 | 108855 | 28:24072970 | 108865 | 28:24222637 | 108933 | 28:25714994 |
| 108982 | 28:26523238 | 109011 | 28:27080357 | 109031 | 28:27474956 | 109048 | 28:27902972 | 109049 | 28:27904071 |
| 109076 | 28:28055097 | 109080 | 28:28119648 | 109110 | 28:28723390 | 109121 | 28:28968643 | 109143 | 28:29407005 |
| 109198 | 28:30842659 | 109208 | 28:31044910 | 109221 | 28:31459804 | 109240 | 28:31905889 | 109252 | 28:32096800 |
| 109278 | 28:32495037 | 109286 | 28:32698642 | 109290 | 28:32777537 | 109291 | 28:32777974 | 109292 | 28:32780910 |
| 109294 | 28:32792000 | 109295 | 28:32793533 | 109297 | 28:32795812 | 109298 | 28:32796515 | 109299 | 28:32801458 |
| 109300 | 28:32803767 | 109301 | 28:32808502 | 109369 | 28:34084210 | 109414 | 28:34925017 | 109415 | 28:34933980 |
| 109437 | 28:35473082 | 109450 | 28:35713729 | 109465 | 28:36047723 | 109466 | 28:36049682 | 109467 | 28:36059895 |
| 109493 | 28:36735400 | 109520 | 28:37458115 | 109529 | 28:37571711 | 109548 | 28:37918674 | 109556 | 28:38086633 |
| 109561 | 28:38273052 | 109564 | 28:38338458 | 109566 | 28:38411536 | 109582 | 28:38715969 | 109594 | 28:39018331 |
| 109627 | 28:39556352 | 109657 | 28:40413981 | 109730 | 28:40820229 | 109739 | 28:40945850 | 109779 | 28:41644978 |
| 109847 | 28:42940481 | 109867 | 28:43165757 | 109965 | 28:45127262 | 109966 | 28:45146124 | 109973 | 28:45229830 |
| 110039 | 29:1136794 | 110043 | 29:1245711 | 110047 | 29:1296904 | 110076 | 29:1876223 | 110091 | 29:2287359 |
| 110092 | 29:2305683 | 110097 | 29:2369666 | 110105 | 29:2449190 | 110106 | 29:2457832 | 110107 | 29:2458133 |
| 110109 | 29:2466616 | 110110 | 29:2475067 | 110111 | 29:2485044 | 110112 | 29:2491210 | 110127 | 29:2791675 |
| 110128 | 29:2792329 | 110131 | 29:2922954 | 110140 | 29:3034446 | 110142 | 29:3054628 | 110156 | 29:3415853 |
| 110167 | 29:3672128 | 110187 | 29:4149425 | 110212 | 29:4771466 | 110214 | 29:4838513 | 110219 | 29:4985399 |
| 110263 | 29:6292926 | 110276 | 29:6759181 | 110307 | 29:7614767 | 110312 | 29:7747910 | 110329 | 29:8183109 |
| 110331 | 29:8211095 | 110341 | 29:8657356 | 110344 | 29:8692066 | 110416 | 29:10237310 | 110425 | 29:10361704 |
| 110438 | 29:10770679 | 110459 | 29:11203337 | 110493 | 29:11981461 | 110533 | 29:12780503 | 110548 | 29:13287093 |
| 110563 | 29:13704499 | 110564 | 29:13711224 | 110582 | 29:14222266 | 110605 | 29:14863923 | 110612 | 29:15023531 |
| 110618 | 29:15136113 | 110630 | 29:15491770 | 110631 | 29:15518673 | 110634 | 29:15577523 | 110637 | 29:15628215 |
| 110639 | 29:15685760 | 110644 | 29:15764983 | 110650 | 29:16007380 | 110653 | 29:16061265 | 110745 | 29:19039096 |
| 110760 | 29:19634491 | 110788 | 29:20300393 | 110792 | 29:20361512 | 110794 | 29:20457751 | 110803 | 29:20716777 |
| 110810 | 29:20812973 | 110811 | 29:20825149 | 110815 | 29:20965169 | 110827 | 29:21317409 | 110828 | 29:21320200 |
| 110829 | 29:21330819 | 110882 | 29:22764191 | 110895 | 29:23102280 | 110921 | 29:23595059 | 110973 | 29:24932130 |
| 110975 | 29:24968087 | 111062 | 29:27477867 | 111077 | 29:27723748 | 111098 | 29:28108791 | 111110 | 29:28257705 |
| 111180 | 29:29705618 | 111248 | 29:30967685 | 111258 | 29:31273255 | 111262 | 29:31338655 | 111365 | 29:33741610 |
| 111366 | 29:33751577 | 111373 | 29:33898078 | 111396 | 29:34412457 | 111400 | 29:34469913 | 111401 | 29:34497844 |
| 111435 | 29:35142257 | 111439 | 29:35191995 | 111471 | 29:35837500 | 111477 | 29:35923286 | 111483 | 29:36087955 |
| 111486 | 29:36106402 | 111506 | 29:36396078 | 111536 | 29:37001499 | 111537 | 29:37010699 | 111557 | 29:37383025 |
| 111568 | 29:37754439 | 111570 | 29:37797892 | 111594 | 29:39272372 | 111606 | 29:39663800 | 111642 | 29:40480123 |
| 111672 | 29:40880222 | 111682 | 29:41078094 | 111692 | 29:41274073 | 111700 | 29:41379850 | 111702 | 29:41422028 |
| 111703 | 29:41439204 | 111707 | 29:41511246 | 111709 | 29:41535408 | 111752 | 29:42380020 | 111803 | 29:43370086 |
| 111852 | 29:44022611 | 111855 | 29:44090714 | 111857 | 29:44103946 | 111858 | 29:44111412 | 111882 | 29:44504806 |
| 111932 | 29:45854845 | 111937 | 29:45910725 | 111971 | 29:46622198 | 112006 | 29:47401443 | 112029 | 29:47889180 |
| 112033 | 29:47969493 | 112061 | 29:48682297 | 112074 | 29:48937511 | 112075 | 29:48937944 | 112076 | 29:48939020 |
| 112078 | 29:49019691 | 112079 | 29:49027895 | 112080 | 29:49027958 | 112081 | 29:49030254 | 112082 | 29:49031741 |
| 112083 | 29:49031778 | 112084 | 29:49032495 | 112085 | 29:49032522 | 112086 | 29:49034373 | 112087 | 29:49034762 |
| 112088 | 29:49036958 | 112089 | 29:49042618 | 112090 | 29:49047332 | 112091 | 29:49047368 | 112093 | 29:49100292 |
| 112095 | 29:49200650 | 112096 | 29:49201144 | 112149 | 29:50487594 | 112150 | 29:50487813 | 112151 | 29:50490270 |
| 112155 | 29:50531220 | | | | | | | | |

In the present disclosure, a second marker panel was a set of functional region markers integrating omics data from three sources. The three sources were: (1) transcriptome data of the longissimus dorsi muscle, liver, and subcutaneous fat of 120 adult cattle. Association analysis was conducted between the genotype of Illumina BovineHD beadchip and the gene expression level as a phenotype. By mapping and locating cis-eQTL and trans-eQTL, a total of 3,800 markers related to the expression of tissue-specific genes were selected. (2) Targeted sequence capture was conducted on all known exons and gene regulatory regions in the bovine genome, and individuals with phenotypic differences in body length and heart girth were selected. After high-throughput sequencing, the capture sequencing data of the high- and low-phenotype groups were obtained separately. A total of 9,243 markers were selected, including 9,242 SNPs in the exon region and 1 Indel that were related to differences between phenotype groups. (3) For the open chromatin region of the genome, ATAC-seq data involved in epigenetics and gene regulation were obtained: at the cellular level, the different stages of myoblast proliferation and differentiation were analyzed, and the difference peak regions in different stages were mapped to the SNP loci of Illumina BovineHD beadchip, such that 5,437 SNPs were obtained; at the individual level, the ATAC-seq data of the longissimus dorsi muscle of adult Huaxi cattle were analyzed, and the differential peak regions were mapped to the resequencing data of 44 Huaxi cattle individuals. A SNP with the highest MAF was selected in each interval, and 4,561 SNPs were obtained. By combining the two parts of ATAC-seq results, 9,985 SNPs involved in epigenetic and gene regulation were selected. Based on the sets of functional regions identified above, a total of 22,938 functional markers from different sources of omics data were obtained through biostatistical analysis and deduplication. The information of second marker panel was shown in Table 2:

TABLE 2
Location information of second marker panel

| NO. | Position | NO. | Position | NO. | Position | NO. | Position | NO. | Position |
|---|---|---|---|---|---|---|---|---|---|
| 23 | 1:1010056 | 74 | 1:1706307 | 78 | 1:1817641 | 79 | 1:1821203 | 81 | 1:1836176 |
| 84 | 1:1886288 | 87 | 1:1979359 | 88 | 1:1997766 | 91 | 1:2032673 | 109 | 1:2304587 |
| 112 | 1:2390062 | 117 | 1:2749113 | 125 | 1:3018314 | 138 | 1:3210484 | 143 | 1:3282090 |
| 153 | 1:3479131 | 158 | 1:3520298 | 174 | 1:3713849 | 175 | 1:3716490 | 177 | 1:3718949 |
| 178 | 1:3720622 | 179 | 1:3723615 | 192 | 1:4021054 | 194 | 1:4058825 | 197 | 1:4081869 |
| 200 | 1:4127064 | 201 | 1:4130636 | 221 | 1:4542229 | 245 | 1:5107763 | 252 | 1:5359288 |
| 274 | 1:5770588 | 312 | 1:6612693 | 320 | 1:6739866 | 328 | 1:6816263 | 329 | 1:6827691 |
| 331 | 1:6856676 | 333 | 1:6963461 | 335 | 1:7027764 | 342 | 1:7064962 | 349 | 1:7173547 |
| 351 | 1:7269335 | 356 | 1:7378233 | 362 | 1:7438240 | 363 | 1:7441167 | 368 | 1:7637339 |
| 391 | 1:8312182 | 395 | 1:8473407 | 406 | 1:8738949 | 420 | 1:9069741 | 422 | 1:9092818 |
| 426 | 1:9259731 | 431 | 1:9403159 | 432 | 1:9442145 | 433 | 1:9442252 | 434 | 1:9442256 |
| 435 | 1:9442802 | 436 | 1:9467212 | 437 | 1:9467322 | 439 | 1:9505564 | 441 | 1:9525931 |
| 449 | 1:9675561 | 450 | 1:9679977 | 451 | 1:9699218 | 456 | 1:9843069 | 460 | 1:9929401 |
| 468 | 1:10048554 | 472 | 1:10225421 | 473 | 1:10225456 | 474 | 1:10231200 | 476 | 1:10264906 |
| 477 | 1:10308984 | 479 | 1:10360268 | 487 | 1:10501157 | 490 | 1:10523191 | 491 | 1:10537240 |
| 494 | 1:10563513 | 495 | 1:10565732 | 498 | 1:10605121 | 502 | 1:10771543 | 503 | 1:10778102 |
| 505 | 1:10809430 | 506 | 1:10823521 | 520 | 1:11223644 | 531 | 1:11472343 | 535 | 1:11550314 |
| 544 | 1:12010431 | 555 | 1:12378491 | 556 | 1:12378514 | 566 | 1:12644235 | 572 | 1:12803499 |
| 573 | 1:12803538 | 574 | 1:12803609 | 584 | 1:13280975 | 585 | 1:13288892 | 587 | 1:13292022 |
| 588 | 1:13297157 | 591 | 1:13398597 | 618 | 1:13725800 | 645 | 1:14655035 | 648 | 1:14720755 |
| 668 | 1:15201042 | 669 | 1:15201103 | 670 | 1:15201137 | 695 | 1:15982024 | 702 | 1:16155554 |
| 703 | 1:16155577 | 704 | 1:16155627 | 705 | 1:16155668 | 714 | 1:16447334 | 723 | 1:16654742 |
| 741 | 1:17350471 | 748 | 1:17481344 | 749 | 1:17481363 | 753 | 1:17580720 | 774 | 1:18459894 |
| 799 | 1:19402363 | 800 | 1:19409456 | 804 | 1:19493132 | 814 | 1:19676162 | 819 | 1:19929109 |
| 833 | 1:20279571 | 837 | 1:20407748 | 840 | 1:20480157 | 845 | 1:20570353 | 847 | 1:20621543 |
| 857 | 1:20823430 | 860 | 1:20921794 | 861 | 1:20974759 | 863 | 1:21005976 | 868 | 1:21167645 |
| 872 | 1:21346196 | 883 | 1:21646634 | 890 | 1:21783397 | 896 | 1:21921230 | 902 | 1:22076454 |
| 925 | 1:22688749 | 933 | 1:22949333 | 947 | 1:23444110 | 952 | 1:23709011 | 953 | 1:23709064 |
| 990 | 1:24753266 | 993 | 1:24782983 | 1010 | 1:25234761 | 1011 | 1:25234832 | 1023 | 1:25497647 |
| 1056 | 1:26533932 | 1058 | 1:26552621 | 1061 | 1:26559576 | 1063 | 1:26611592 | 1067 | 1:26755277 |
| 1070 | 1:26880560 | 1072 | 1:26882959 | 1077 | 1:26969554 | 1082 | 1:27132369 | 1084 | 1:27171038 |
| 1092 | 1:27401121 | 1134 | 1:29068209 | 1144 | 1:29340605 | 1147 | 1:29403350 | 1151 | 1:29458506 |
| 1158 | 1:29712048 | 1165 | 1:29884320 | 1170 | 1:29949770 | 1180 | 1:30194782 | 1201 | 1:30897798 |
| 1219 | 1:31546632 | 1227 | 1:31736868 | 1249 | 1:32659120 | 1262 | 1:33136711 | 1265 | 1:33292309 |
| 1270 | 1:33496557 | 1272 | 1:33533012 | 1289 | 1:33884832 | 1300 | 1:34174940 | 1309 | 1:34602877 |
| 1311 | 1:34663000 | 1313 | 1:34664612 | 1317 | 1:34824821 | 1318 | 1:34824930 | 1321 | 1:34924925 |
| 1324 | 1:35000632 | 1331 | 1:35082343 | 1344 | 1:35345367 | 1349 | 1:35407003 | 1362 | 1:35733756 |
| 1370 | 1:36018590 | 1372 | 1:36111561 | 1379 | 1:36303126 | 1389 | 1:36436645 | 1392 | 1:36459421 |
| 1393 | 1:36459510 | 1402 | 1:36697707 | 1408 | 1:36828697 | 1414 | 1:37032363 | 1415 | 1:37032440 |
| 1426 | 1:37411283 | 1428 | 1:37442242 | 1434 | 1:37638872 | 1438 | 1:37686047 | 1440 | 1:37725772 |
| 1450 | 1:37956983 | 1453 | 1:38013891 | 1468 | 1:38540153 | 1469 | 1:38599466 | 1476 | 1:38720880 |
| 1494 | 1:39249574 | 1505 | 1:39697516 | 1534 | 1:40506922 | 1538 | 1:40629765 | 1548 | 1:40789010 |
| 1573 | 1:41686185 | 1579 | 1:41784113 | 1580 | 1:41788052 | 1581 | 1:41795018 | 1582 | 1:41795589 |
| 1583 | 1:41796750 | 1584 | 1:41797989 | 1586 | 1:41805753 | 1587 | 1:41835927 | 1588 | 1:41836033 |
| 1590 | 1:41886688 | 1600 | 1:42107409 | 1601 | 1:42131473 | 1604 | 1:42159211 | 1605 | 1:42170788 |
| 1609 | 1:42224416 | 1611 | 1:42265122 | 1612 | 1:42285326 | 1613 | 1:42293500 | 1619 | 1:42519517 |
| 1633 | 1:42917734 | 1640 | 1:43141355 | 1643 | 1:43211702 | 1645 | 1:43225435 | 1647 | 1:43292695 |
| 1652 | 1:43443436 | 1656 | 1:43496586 | 1668 | 1:43756861 | 1670 | 1:43772333 | 1672 | 1:43787631 |
| 1684 | 1:44030562 | 1686 | 1:44124010 | 1688 | 1:44137020 | 1690 | 1:44236960 | 1700 | 1:44393495 |
| 1701 | 1:44422846 | 1708 | 1:44623944 | 1712 | 1:44766328 | 1714 | 1:44820415 | 1715 | 1:44820480 |
| 1716 | 1:44820576 | 1717 | 1:44847495 | 1732 | 1:44947997 | 1733 | 1:44956505 | 1734 | 1:44959367 |
| 1735 | 1:44960388 | 1737 | 1:44995524 | 1738 | 1:44998217 | 1740 | 1:45030214 | 1741 | 1:45036157 |
| 1744 | 1:45061133 | 1748 | 1:45196717 | 1750 | 1:45216991 | 1759 | 1:45365527 | 1760 | 1:45372759 |
| 1784 | 1:46185873 | 1793 | 1:46488716 | 1821 | 1:47439819 | 1822 | 1:47457097 | 1823 | 1:47465147 |
| 1829 | 1:47631393 | 1852 | 1:48538887 | 1853 | 1:48538897 | 1866 | 1:48823158 | 1893 | 1:49844520 |
| 1907 | 1:50327235 | 1913 | 1:50531202 | 1916 | 1:50627944 | 1917 | 1:50631061 | 1918 | 1:50631100 |
| 1919 | 1:50637655 | 1924 | 1:50846942 | 1925 | 1:50856429 | 1926 | 1:50874802 | 1928 | 1:50934316 |
| 1931 | 1:50978540 | 1934 | 1:51048823 | 1945 | 1:51501180 | 1958 | 1:51956854 | 1960 | 1:52001408 |
| 1974 | 1:52418784 | 1975 | 1:52420721 | 1977 | 1:52468623 | 1979 | 1:52499614 | 1980 | 1:52499623 |
| 1982 | 1:52592314 | 1983 | 1:52616071 | 1984 | 1:52639898 | 1989 | 1:52845116 | 1999 | 1:53149622 |
| 2008 | 1:53293355 | 2011 | 1:53338531 | 2015 | 1:53534215 | 2034 | 1:53685551 | 2036 | 1:53685641 |
| 2037 | 1:53685662 | 2038 | 1:53685710 | 2043 | 1:53760822 | 2049 | 1:53972219 | 2055 | 1:54092473 |
| 2056 | 1:54092637 | 2057 | 1:54092679 | 2058 | 1:54093927 | 2059 | 1:54101343 | 2068 | 1:54343019 |
| 2076 | 1:54460688 | 2088 | 1:54757168 | 2089 | 1:54766036 | 2096 | 1:55026386 | 2097 | 1:55026433 |
| 2108 | 1:55421744 | 2110 | 1:55482490 | 2121 | 1:55927365 | 2122 | 1:55964487 | 2123 | 1:55970922 |
| 2132 | 1:56253478 | 2138 | 1:56370458 | 2142 | 1:56472558 | 2145 | 1:56498727 | 2147 | 1:56540591 |
| 2154 | 1:56759097 | 2162 | 1:56937696 | 2165 | 1:56975870 | 2170 | 1:57137491 | 2173 | 1:57217658 |
| 2174 | 1:57217807 | 2175 | 1:57251079 | 2176 | 1:57266134 | 2178 | 1:57309255 | 2181 | 1:57398938 |
| 2184 | 1:57490618 | 2185 | 1:57560306 | 2197 | 1:57870387 | 2198 | 1:57917830 | 2203 | 1:58030359 |
| 2205 | 1:58075432 | 2206 | 1:58075463 | 2207 | 1:58075499 | 2208 | 1:58075521 | 2209 | 1:58078354 |
| 2210 | 1:58078466 | 2211 | 1:58097510 | 2221 | 1:58305398 | 2222 | 1:58305455 | 2223 | 1:58315322 |
| 2224 | 1:58315443 | 2225 | 1:58315990 | 2231 | 1:58540410 | 2232 | 1:58618667 | 2236 | 1:58750747 |
| 2237 | 1:58773333 | 2240 | 1:58818870 | 2250 | 1:59034012 | 2252 | 1:59104762 | 2258 | 1:59200011 |
| 2259 | 1:59203569 | 2263 | 1:59371385 | 2270 | 1:59656138 | 2279 | 1:59830679 | 2293 | 1:60123244 |
| 2318 | 1:60784872 | 2325 | 1:60918912 | 2330 | 1:61097118 | 2333 | 1:61128757 | 2336 | 1:61160140 |
| 2369 | 1:62066946 | 2370 | 1:62092465 | 2386 | 1:62457515 | 2399 | 1:62621737 | 2402 | 1:62637571 |
| 2406 | 1:62817788 | 2407 | 1:62817866 | 2428 | 1:63139698 | 2438 | 1:63427104 | 2440 | 1:63476336 |
| 2441 | 1:63517812 | 2451 | 1:63851933 | 2456 | 1:64008039 | 2461 | 1:64183671 | 2463 | 1:64200486 |
| 2464 | 1:64200539 | 2466 | 1:64262468 | 2477 | 1:64431800 | 2478 | 1:64462597 | 2479 | 1:64462675 |
| 2480 | 1:64463152 | 2481 | 1:64463254 | 2482 | 1:64467138 | 2483 | 1:64467211 | 2486 | 1:64505435 |
| 2496 | 1:64824564 | 2502 | 1:64940185 | 2509 | 1:65157725 | 2512 | 1:65199200 | 2513 | 1:65216449 |
| 2514 | 1:65224381 | 2515 | 1:65228988 | 2516 | 1:65239311 | 2520 | 1:65377133 | 2545 | 1:66100053 |
| 2547 | 1:66136711 | 2557 | 1:66502772 | 2558 | 1:66511698 | 2559 | 1:66515809 | 2561 | 1:66547988 |
| 2569 | 1:66694865 | 2581 | 1:67173187 | 2588 | 1:67320099 | 2590 | 1:67331681 | 2591 | 1:67411736 |
| 2593 | 1:67432038 | 2594 | 1:67439085 | 2596 | 1:67473778 | 2604 | 1:67760033 | 2608 | 1:67861511 |
| 2618 | 1:68238481 | 2619 | 1:68259739 | 2623 | 1:68332459 | 2627 | 1:68449381 | 2628 | 1:68488305 |
| 2630 | 1:68501205 | 2631 | 1:68517809 | 2635 | 1:68649543 | 2637 | 1:68680831 | 2638 | 1:68682422 |
| 2639 | 1:68682461 | 2640 | 1:68690274 | 2644 | 1:68784544 | 2646 | 1:68792494 | 2647 | 1:68793488 |
| 2648 | 1:68793540 | 2649 | 1:68793542 | 2650 | 1:68793543 | 2651 | 1:68797693 | 2656 | 1:68869228 |
| 2663 | 1:68996534 | 2668 | 1:69017745 | 2670 | 1:69042317 | 2671 | 1:69047274 | 2672 | 1:69047282 |
| 2673 | 1:69047320 | 2679 | 1:69124262 | 2682 | 1:69203913 | 2684 | 1:69217319 | 2685 | 1:69217672 |
| 2698 | 1:69582986 | 2699 | 1:69585403 | 2704 | 1:69764910 | 2712 | 1:69883156 | 2717 | 1:69982394 |
| 2720 | 1:70106968 | 2725 | 1:70231031 | 2726 | 1:70231761 | 2727 | 1:70248171 | 2730 | 1:70343458 |
| 2732 | 1:70365636 | 2734 | 1:70390927 | 2741 | 1:70466635 | 2742 | 1:70466719 | 2743 | 1:70466765 |
| 2744 | 1:70468358 | 2745 | 1:70473449 | 2746 | 1:70473451 | 2747 | 1:70473452 | 2748 | 1:70473471 |
| 2749 | 1:70473493 | 2750 | 1:70485290 | 2751 | 1:70485353 | 2753 | 1:70494055 | 2754 | 1:70497518 |
| 2755 | 1:70500089 | 2762 | 1:70616796 | 2771 | 1:70783887 | 2772 | 1:70791872 | 2773 | 1:70801580 |
| 2790 | 1:71415748 | 2791 | 1:71416509 | 2792 | 1:71416751 | 2793 | 1:71416881 | 2794 | 1:71416890 |
| 2803 | 1:71609698 | 2817 | 1:72069890 | 2819 | 1:72081243 | 2820 | 1:72090597 | 2834 | 1:72454020 |
| 2836 | 1:72484009 | 2839 | 1:72519103 | 2849 | 1:72679096 | 2850 | 1:72679109 | 2851 | 1:72679167 |
| 2854 | 1:72714931 | 2855 | 1:72726681 | 2856 | 1:72732857 | 2859 | 1:72803314 | 2863 | 1:72952213 |
| 2864 | 1:72955756 | 2866 | 1:73093141 | 2871 | 1:73187152 | 2872 | 1:73207902 | 2879 | 1:73477972 |
| 2883 | 1:73591733 | 2887 | 1:73663050 | 2888 | 1:73671409 | 2890 | 1:73744599 | 2898 | 1:73902125 |
| 2902 | 1:73954764 | 2908 | 1:74183606 | 2915 | 1:74460825 | 2917 | 1:74460955 | 2918 | 1:74489282 |
| 2919 | 1:74490359 | 2922 | 1:74553801 | 2935 | 1:74976635 | 2957 | 1:75592224 | 2960 | 1:75682742 |
| 2967 | 1:75798251 | 2968 | 1:75798252 | 2969 | 1:75798317 | 2972 | 1:75852855 | 2980 | 1:76051714 |
| 3013 | 1:77045567 | 3020 | 1:77255234 | 3029 | 1:77397999 | 3032 | 1:77443583 | 3033 | 1:77469880 |
| 3036 | 1:77593114 | 3038 | 1:77616604 | 3040 | 1:77652308 | 3044 | 1:77698757 | 3048 | 1:77727505 |
| 3054 | 1:77958907 | 3074 | 1:78375438 | 3077 | 1:78445734 | 3082 | 1:78556250 | 3083 | 1:78566682 |
| 3093 | 1:78911959 | 3095 | 1:78989138 | 3098 | 1:79082187 | 3101 | 1:79138960 | 3102 | 1:79169504 |
| 3105 | 1:79213373 | 3107 | 1:79242860 | 3110 | 1:79298445 | 3114 | 1:79357690 | 3118 | 1:79485282 |
| 3123 | 1:79562994 | 3156 | 1:80023498 | 3161 | 1:80069552 | 3163 | 1:80074105 | 3172 | 1:80391190 |
| 3173 | 1:80413261 | 3183 | 1:80685030 | 3192 | 1:80940339 | 3196 | 1:81028408 | 3201 | 1:81051808 |
| 3208 | 1:81170302 | 3209 | 1:81183483 | 3215 | 1:81348584 | 3229 | 1:81706937 | 3235 | 1:81878324 |
| 3240 | 1:82041541 | 3241 | 1:82041559 | 3242 | 1:82047704 | 3246 | 1:82149539 | 3258 | 1:82473626 |
| 3264 | 1:82519837 | 3265 | 1:82541328 | 3266 | 1:82541450 | 3268 | 1:82558245 | 3273 | 1:82643904 |
| 3276 | 1:82705721 | 3278 | 1:82758391 | 3284 | 1:83070275 | 3285 | 1:83097205 | 3286 | 1:83133001 |
| 3291 | 1:83278833 | 3292 | 1:83284140 | 3293 | 1:83330542 | 3306 | 1:83810343 | 3310 | 1:83974373 |
| 3312 | 1:84000147 | 3315 | 1:84047005 | 3316 | 1:84058598 | 3338 | 1:85042289 | 3344 | 1:85087002 |
| 3346 | 1:85130568 | 3368 | 1:85947378 | 3369 | 1:85948692 | 3371 | 1:85976228 | 3374 | 1:86122181 |
| 3375 | 1:86146073 | 3376 | 1:86156408 | 3377 | 1:86160591 | 3378 | 1:86177464 | 3379 | 1:86185749 |
| 3380 | 1:86186427 | 3394 | 1:86631754 | 3406 | 1:86967586 | 3420 | 1:87399098 | 3421 | 1:87399338 |
| 3422 | 1:87414899 | 3428 | 1:87574019 | 3431 | 1:87648729 | 3434 | 1:87752223 | 3441 | 1:88012422 |
| 3467 | 1:88872312 | 3468 | 1:88874634 | 3469 | 1:88880495 | 3471 | 1:88896168 | 3472 | 1:88908213 |
| 3474 | 1:88987015 | 3476 | 1:89086745 | 3478 | 1:89097927 | 3479 | 1:89098024 | 3482 | 1:89224144 |
| 3484 | 1:89300003 | 3486 | 1:89343474 | 3487 | 1:89349576 | 3488 | 1:89383250 | 3492 | 1:89498954 |
| 3494 | 1:89539395 | 3495 | 1:89539442 | 3508 | 1:89796658 | 3509 | 1:89799413 | 3522 | 1:90043900 |
| 3524 | 1:90075379 | 3550 | 1:90588788 | 3552 | 1:90615269 | 3554 | 1:90648889 | 3576 | 1:91255865 |
| 3577 | 1:91285295 | 3593 | 1:91706481 | 3596 | 1:91737921 | 3597 | 1:91739554 | 3602 | 1:91928880 |
| 3609 | 1:92184004 | 3623 | 1:92503312 | 3668 | 1:93883957 | 3670 | 1:93930496 | 3674 | 1:94035144 |
| 3675 | 1:94035267 | 3680 | 1:94131411 | 3681 | 1:94134092 | 3699 | 1:94576910 | 3709 | 1:94877027 |
| 3729 | 1:95423137 | 3730 | 1:95439594 | 3731 | 1:95455255 | 3733 | 1:95478530 | 3734 | 1:95494762 |
| 3745 | 1:95806271 | 3747 | 1:95893257 | 3752 | 1:96002349 | 3754 | 1:96026129 | 3756 | 1:96084552 |
| 3757 | 1:96085021 | 3760 | 1:96180861 | 3764 | 1:96218350 | 3765 | 1:96220152 | 3770 | 1:96344874 |
| 3779 | 1:96474332 | 3784 | 1:96640052 | 3801 | 1:97152251 | 3819 | 1:97537514 | 3832 | 1:97878133 |
| 3843 | 1:98148099 | 3847 | 1:98313182 | 3857 | 1:98673491 | 3867 | 1:98891260 | 3876 | 1:99200317 |
| 3882 | 1:99297524 | 3883 | 1:99323405 | 3886 | 1:99418906 | 3890 | 1:99501232 | 3906 | 1:99941713 |
| 3914 | 1:100198782 | 3917 | 1:100296109 | 3919 | 1:100321230 | 3927 | 1:100567937 | 3948 | 1:101273216 |
| 3969 | 1:101894473 | 3980 | 1:102388029 | 3981 | 1:102388112 | 3988 | 1:102582187 | 3990 | 1:102591751 |
| 3993 | 1:102638499 | 3995 | 1:102646032 | 3997 | 1:102698749 | 4004 | 1:102911722 | 4008 | 1:103023798 |
| 4037 | 1:104209003 | 4038 | 1:104263712 | 4045 | 1:104522827 | 4053 | 1:104831357 | 4058 | 1:104915762 |
| 4060 | 1:104987991 | 4061 | 1:105015424 | 4074 | 1:105367807 | 4079 | 1:105447199 | 4103 | 1:106048772 |
| 4108 | 1:106226124 | 4111 | 1:106284785 | 4123 | 1:106497025 | 4144 | 1:107326886 | 4149 | 1:107424074 |
| 4153 | 1:107500490 | 4154 | 1:107538852 | 4155 | 1:107544465 | 4156 | 1:107544536 | 4161 | 1:107781031 |
| 4164 | 1:107863018 | 4170 | 1:108114016 | 4189 | 1:108515165 | 4191 | 1:108556182 | 4192 | 1:108556227 |
| 4203 | 1:108851719 | 4204 | 1:108851745 | 4205 | 1:108853578 | 4206 | 1:108854832 | 4207 | 1:108870778 |
| 4208 | 1:108874776 | 4209 | 1:108881505 | 4210 | 1:108884859 | 4211 | 1:108894708 | 4212 | 1:108894728 |
| 4213 | 1:108896221 | 4230 | 1:109527893 | 4231 | 1:109527919 | 4245 | 1:109820651 | 4250 | 1:109870082 |
| 4266 | 1:110126279 | 4267 | 1:110175621 | 4269 | 1:110196398 | 4273 | 1:110293441 | 4276 | 1:110341970 |
| 4277 | 1:110342054 | 4278 | 1:110342111 | 4283 | 1:110476863 | 4288 | 1:110573226 | 4289 | 1:110590869 |
| 4292 | 1:110633655 | 4293 | 1:110653778 | 4295 | 1:110665646 | 4296 | 1:110666740 | 4302 | 1:110816709 |
| 4309 | 1:111186619 | 4310 | 1:111194151 | 4311 | 1:111199408 | 4312 | 1:111247418 | 4313 | 1:111268099 |
| 4325 | 1:111708777 | 4327 | 1:111748641 | 4331 | 1:111775768 | 4332 | 1:111775805 | 4333 | 1:111798208 |
| 4345 | 1:112208772 | 4353 | 1:112368514 | 4354 | 1:112398275 | 4355 | 1:112400081 | 4361 | 1:112555088 |
| 4362 | 1:112555094 | 4363 | 1:112555107 | 4364 | 1:112555108 | 4365 | 1:112555300 | 4366 | 1:112555322 |
| 4367 | 1:112555340 | 4368 | 1:112555460 | 4369 | 1:112555492 | 4370 | 1:112556677 | 4371 | 1:112556716 |
| 4372 | 1:112556776 | 4375 | 1:112581875 | 4376 | 1:112581930 | 4377 | 1:112581975 | 4378 | 1:112595293 |
| 4400 | 1:113354543 | 4403 | 1:113365903 | 4404 | 1:113369018 | 4405 | 1:113385595 | 4406 | 1:113385624 |
| 4407 | 1:113385658 | 4417 | 1:113584302 | 4457 | 1:114467455 | 4460 | 1:114596976 | 4462 | 1:114728742 |
| 4465 | 1:114816068 | 4468 | 1:114840022 | 4488 | 1:115324305 | 4490 | 1:115377104 | 4491 | 1:115413995 |
| 4496 | 1:115579666 | 4502 | 1:115790600 | 4528 | 1:116754887 | 4532 | 1:116818074 | 4533 | 1:116821719 |
| 4535 | 1:116860895 | 4538 | 1:116891857 | 4540 | 1:117003117 | 4546 | 1:117113278 | 4556 | 1:117319749 |
| 4563 | 1:117420923 | 4564 | 1:117421250 | 4576 | 1:117738265 | 4578 | 1:117758868 | 4586 | 1:117887255 |
| 4587 | 1:117897860 | 4588 | 1:117912537 | 4589 | 1:117935755 | 4591 | 1:117945817 | 4597 | 1:118048415 |
| 4598 | 1:118085158 | 4600 | 1:118114537 | 4602 | 1:118133789 | 4605 | 1:118196612 | 4608 | 1:118214654 |
| 4609 | 1:118227302 | 4610 | 1:118256496 | 4611 | 1:118257517 | 4612 | 1:118259606 | 4613 | 1:118273499 |
| 4614 | 1:118276261 | 4615 | 1:118276735 | 4616 | 1:118277267 | 4617 | 1:118278387 | 4618 | 1:118284875 |
| 4619 | 1:118286166 | 4620 | 1:118288018 | 4621 | 1:118290177 | 4622 | 1:118295131 | 4635 | 1:118530178 |
| 4636 | 1:118544527 | 4642 | 1:118690300 | 4643 | 1:118690309 | 4652 | 1:118962324 | 4654 | 1:118985100 |
| 4665 | 1:119176913 | 4666 | 1:119189605 | 4667 | 1:119210631 | 4672 | 1:119350100 | 4683 | 1:119561515 |
| 4684 | 1:119561537 | 4685 | 1:119561564 | 4701 | 1:120064732 | 4712 | 1:120386879 | 4722 | 1:120500835 |
| 4726 | 1:120563845 | 4760 | 1:121647921 | 4762 | 1:121707542 | 4771 | 1:121946641 | 4781 | 1:122232742 |
| 4785 | 1:122286938 | 4786 | 1:122287045 | 4790 | 1:122500672 | 4794 | 1:122540031 | 4799 | 1:122654668 |
| 4801 | 1:122686036 | 4807 | 1:122783659 | 4808 | 1:122784649 | 4810 | 1:122849903 | 4811 | 1:122901551 |
| 4814 | 1:123034294 | 4819 | 1:123266689 | 4821 | 1:123270508 | 4827 | 1:123475726 | 4835 | 1:123679492 |
| 4836 | 1:123692405 | 4837 | 1:123706359 | 4838 | 1:123709988 | 4839 | 1:123720581 | 4848 | 1:124014834 |
| 4854 | 1:124194081 | 4860 | 1:124271732 | 4861 | 1:124271768 | 4862 | 1:124271786 | 4863 | 1:124314731 |
| 4864 | 1:124324829 | 4865 | 1:124331752 | 4866 | 1:124343187 | 4870 | 1:124506907 | 4871 | 1:124528147 |
| 4878 | 1:124666898 | 4879 | 1:124672817 | 4886 | 1:124887464 | 4891 | 1:124948606 | 4894 | 1:124980393 |
| 4895 | 1:124980562 | 4898 | 1:125121982 | 4902 | 1:125269301 | 4904 | 1:125360929 | 4905 | 1:125363719 |
| 4907 | 1:125382225 | 4908 | 1:125386885 | 4913 | 1:125521147 | 4919 | 1:125583210 | 4920 | 1:125608898 |
| 4922 | 1:125609041 | 4928 | 1:125643614 | 4944 | 1:125851156 | 4946 | 1:125931085 | 4951 | 1:126050865 |
| 4953 | 1:126166588 | 4954 | 1:126167017 | 4955 | 1:126188011 | 4962 | 1:126320901 | 4970 | 1:126452685 |
| 4979 | 1:126828386 | 4981 | 1:126867108 | 4989 | 1:127247333 | 4997 | 1:127448104 | 5001 | 1:127536751 |
| 5004 | 1:127609705 | 5007 | 1:127732186 | 5010 | 1:127791769 | 5011 | 1:127843966 | 5015 | 1:127884865 |
| 5020 | 1:127963540 | 5022 | 1:128091201 | 5032 | 1:128267812 | 5037 | 1:128374395 | 5042 | 1:128512081 |
| 5073 | 1:129150388 | 5080 | 1:129402104 | 5081 | 1:129418328 | 5089 | 1:129590492 | 5090 | 1:129591720 |
| 5091 | 1:129592529 | 5095 | 1:129754416 | 5127 | 1:130632012 | 5128 | 1:130658736 | 5130 | 1:130720354 |
| 5131 | 1:130728433 | 5134 | 1:130792978 | 5137 | 1:130920062 | 5140 | 1:130954555 | 5179 | 1:131419104 |
| 5222 | 1:131860577 | 5242 | 1:132473942 | 5253 | 1:132876761 | 5264 | 1:133379843 | 5265 | 1:133384149 |
| 5266 | 1:133394080 | 5271 | 1:133540327 | 5272 | 1:133591491 | 5289 | 1:134186323 | 5294 | 1:134285791 |
| 5298 | 1:134384145 | 5300 | 1:134413818 | 5301 | 1:134413821 | 5306 | 1:134477738 | 5324 | 1:134948789 |
| 5325 | 1:134961346 | 5327 | 1:134973908 | 5329 | 1:135015189 | 5331 | 1:135049866 | 5337 | 1:135208575 |
| 5341 | 1:135315701 | 5342 | 1:135345248 | 5346 | 1:135410192 | 5347 | 1:135414049 | 5360 | 1:135642886 |
| 5361 | 1:135643955 | 5362 | 1:135644811 | 5363 | 1:135653473 | 5366 | 1:135665914 | 5367 | 1:135666200 |
| 5368 | 1:135666237 | 5369 | 1:135666283 | 5370 | 1:135670574 | 5371 | 1:135672004 | 5372 | 1:135694251 |
| 5374 | 1:135753894 | 5375 | 1:135753940 | 5376 | 1:135753983 | 5377 | 1:135754044 | 5378 | 1:135754264 |
| 5379 | 1:135754300 | 5380 | 1:135758284 | 5381 | 1:135758290 | 5382 | 1:135758332 | 5383 | 1:135762033 |
| 5384 | 1:135763704 | 5385 | 1:135769522 | 5397 | 1:135880893 | 5399 | 1:135916803 | 5400 | 1:135927623 |
| 5401 | 1:135972509 | 5402 | 1:135974364 | 5405 | 1:136033754 | 5406 | 1:136047835 | 5413 | 1:136204362 |
| 5415 | 1:136311162 | 5427 | 1:136734614 | 5434 | 1:137049621 | 5438 | 1:137106364 | 5443 | 1:137294127 |
| 5472 | 1:138643152 | 5473 | 1:138647040 | 5477 | 1:138728014 | 5478 | 1:138739021 | 5483 | 1:138798912 |
| 5489 | 1:138994500 | 5498 | 1:139184610 | 5505 | 1:139411806 | 5508 | 1:139449113 | 5521 | 1:139899458 |
| 5536 | 1:140266541 | 5545 | 1:140543741 | 5554 | 1:140754935 | 5555 | 1:140755072 | 5558 | 1:140876675 |
| 5575 | 1:141145402 | 5579 | 1:141316735 | 5580 | 1:141319127 | 5581 | 1:141319994 | 5583 | 1:141339227 |
| 5584 | 1:141345591 | 5585 | 1:141362575 | 5586 | 1:141364352 | 5587 | 1:141374294 | 5588 | 1:141376997 |
| 5590 | 1:141395382 | 5591 | 1:141421683 | 5597 | 1:141511318 | 5601 | 1:141610105 | 5602 | 1:141610924 |
| 5604 | 1:141627871 | 5606 | 1:141628909 | 5608 | 1:141649600 | 5610 | 1:141651362 | 5611 | 1:141653130 |
| 5612 | 1:141653134 | 5613 | 1:141654271 | 5614 | 1:141654547 | 5615 | 1:141654551 | 5617 | 1:141667906 |
| 5622 | 1:141734030 | 5623 | 1:141741068 | 5625 | 1:141823289 | 5633 | 1:142000532 | 5641 | 1:142239516 |
| 5643 | 1:142251697 | 5651 | 1:142362891 | 5654 | 1:142393561 | 5657 | 1:142478032 | 5659 | 1:142489032 |
| 5667 | 1:142635123 | 5672 | 1:142787751 | 5693 | 1:143128593 | 5700 | 1:143227118 | 5702 | 1:143334405 |
| 5706 | 1:143406113 | 5711 | 1:143569447 | 5712 | 1:143579707 | 5713 | 1:143582830 | 5714 | 1:143588692 |
| 5715 | 1:143591196 | 5716 | 1:143593029 | 5717 | 1:143594628 | 5726 | 1:143746215 | 5728 | 1:143749139 |
| 5729 | 1:143752954 | 5747 | 1:144050118 | 5748 | 1:144050120 | 5757 | 1:144333701 | 5759 | 1:144372951 |
| 5763 | 1:144435905 | 5772 | 1:144635480 | 5776 | 1:144757986 | 5780 | 1:144787067 | 5781 | 1:144809797 |
| 5786 | 1:144894365 | 5794 | 1:145080672 | 5797 | 1:145128073 | 5799 | 1:145136789 | 5802 | 1:145176107 |
| 5804 | 1:145282436 | 5813 | 1:145459919 | 5821 | 1:145623145 | 5822 | 1:145623795 | 5825 | 1:145673098 |
| 5826 | 1:145677709 | 5834 | 1:145827286 | 5835 | 1:145830272 | 5837 | 1:145860541 | 5838 | 1:145860568 |
| 5839 | 1:145866834 | 5840 | 1:145866900 | 5841 | 1:145873941 | 5842 | 1:145873979 | 5843 | 1:145874030 |
| 5848 | 1:145951072 | 5849 | 1:145963833 | 5850 | 1:145968413 | 5852 | 1:145972525 | 5853 | 1:145975945 |
| 5854 | 1:145977236 | 5862 | 1:146052159 | 5871 | 1:146351589 | 5874 | 1:146420270 | 5892 | 1:146975928 |
| 5894 | 1:147008926 | 5899 | 1:147098097 | 5902 | 1:147122843 | 5918 | 1:147397092 | 5919 | 1:147417101 |
| 5921 | 1:147431978 | 5927 | 1:147563989 | 5941 | 1:147846791 | 5946 | 1:147903408 | 5978 | 1:148430252 |
| 5979 | 1:148466864 | 5981 | 1:148508063 | 5982 | 1:148508133 | 5984 | 1:148513206 | 5985 | 1:148549091 |
| 5994 | 1:148736098 | 6000 | 1:148871697 | 6004 | 1:148964961 | 6010 | 1:149177892 | 6011 | 1:149189606 |
| 6014 | 1:149317573 | 6015 | 1:149320003 | 6016 | 1:149323616 | 6017 | 1:149324955 | 6018 | 1:149328203 |
| 6019 | 1:149340128 | 6020 | 1:149342542 | 6021 | 1:149348963 | 6022 | 1:149351545 | 6023 | 1:149358100 |
| 6024 | 1:149360084 | 6025 | 1:149362014 | 6026 | 1:149366388 | 6027 | 1:149367120 | 6028 | 1:149368535 |
| 6029 | 1:149371783 | 6030 | 1:149374604 | 6031 | 1:149375645 | 6032 | 1:149376574 | 6033 | 1:149378550 |
| 6034 | 1:149379934 | 6035 | 1:149380878 | 6036 | 1:149381954 | 6037 | 1:149390100 | 6042 | 1:149443669 |
| 6049 | 1:149642436 | 6065 | 1:149898235 | 6066 | 1:149902664 | 6068 | 1:149929034 | 6077 | 1:150129027 |
| 6078 | 1:150129035 | 6079 | 1:150129044 | 6080 | 1:150129048 | 6081 | 1:150144069 | 6083 | 1:150151876 |
| 6093 | 1:150440522 | 6106 | 1:150701995 | 6109 | 1:150741379 | 6119 | 1:151001062 | 6122 | 1:151048846 |
| 6124 | 1:151109660 | 6134 | 1:151324257 | 6135 | 1:151326467 | 6148 | 1:151556745 | 6149 | 1:151560943 |
| 6150 | 1:151561380 | 6154 | 1:151623827 | 6158 | 1:151643959 | 6161 | 1:151668710 | 6162 | 1:151671521 |
| 6163 | 1:151673956 | 6164 | 1:151675356 | 6165 | 1:151677189 | 6182 | 1:151922660 | 6186 | 1:152015544 |
| 6189 | 1:152071824 | 6215 | 1:152347862 | 6235 | 1:152417708 | 6237 | 1:152422584 | 6238 | 1:152428562 |
| 6249 | 1:152632298 | 6252 | 1:152678335 | 6254 | 1:152745164 | 6260 | 1:152826141 | 6279 | 1:153250410 |
| 6301 | 1:153688732 | 6308 | 1:153828051 | 6322 | 1:154004623 | 6323 | 1:154009064 | 6324 | 1:154009137 |
| 6325 | 1:154009145 | 6327 | 1:154019636 | 6329 | 1:154038751 | 6341 | 1:154370113 | 6358 | 1:154858681 |
| 6388 | 1:155496781 | 6400 | 1:155679617 | 6401 | 1:155679654 | 6402 | 1:155679691 | 6403 | 1:155679758 |
| 6404 | 1:155679762 | 6407 | 1:155716904 | 6429 | 1:156040352 | 6434 | 1:156108198 | 6469 | 1:156749778 |
| 6502 | 1:157422591 | 6505 | 1:157455223 | 6508 | 1:157589021 | 6514 | 1:157988428 | 6517 | 1:158029386 |
| 6518 | 1:158031759 | 6521 | 1:158140847 | 6522 | 1:158227949 | 6550 | 2:773366 | 6570 | 2:1116337 |
| 6571 | 2:1120855 | 6576 | 2:1174595 | 6579 | 2:1234786 | 6580 | 2:1239635 | 6584 | 2:1280967 |
| 6585 | 2:1281046 | 6586 | 2:1286624 | 6587 | 2:1286642 | 6588 | 2:1286747 | 6607 | 2:1669475 |
| 6615 | 2:1857324 | 6616 | 2:1859648 | 6623 | 2:1996597 | 6642 | 2:2733310 | 6649 | 2:2936048 |
| 6657 | 2:3205071 | 6671 | 2:3644419 | 6679 | 2:3866542 | 6680 | 2:3866584 | 6688 | 2:4033904 |
| 6692 | 2:4056030 | 6693 | 2:4056116 | 6702 | 2:4244343 | 6705 | 2:4311599 | 6706 | 2:4311602 |
| 6707 | 2:4311756 | 6708 | 2:4318724 | 6710 | 2:4360783 | 6719 | 2:4631707 | 6720 | 2:4634839 |
| 6721 | 2:4634862 | 6722 | 2:4634886 | 6723 | 2:4634902 | 6724 | 2:4634989 | 6725 | 2:4655242 |
| 6727 | 2:4687767 | 6731 | 2:4782801 | 6749 | 2:5105483 | 6754 | 2:5312133 | 6757 | 2:5343162 |
| 6760 | 2:5372449 | 6767 | 2:5418497 | 6770 | 2:5433956 | 6771 | 2:5437701 | 6776 | 2:5564286 |
| 6778 | 2:5598208 | 6780 | 2:5605765 | 6781 | 2:5668859 | 6786 | 2:5824441 | 6806 | 2:6219961 |
| 6807 | 2:6220338 | 6820 | 2:6413902 | 6826 | 2:6665981 | 6827 | 2:6670529 | 6828 | 2:6670740 |
| 6829 | 2:6670761 | 6830 | 2:6670813 | 6831 | 2:6670846 | 6842 | 2:6830098 | 6844 | 2:6891780 |
| 6846 | 2:6946227 | 6851 | 2:7030426 | 6852 | 2:7053225 | 6855 | 2:7145809 | 6859 | 2:7201484 |
| 6860 | 2:7214741 | 6861 | 2:7228554 | 6865 | 2:7278045 | 6867 | 2:7321689 | 6869 | 2:7334093 |
| 6870 | 2:7339046 | 6871 | 2:7346647 | 6874 | 2:7389988 | 6876 | 2:7414723 | 6877 | 2:7487693 |
| 6880 | 2:7550253 | 6893 | 2:7824299 | 6894 | 2:7854802 | 6895 | 2:7854894 | 6896 | 2:7854898 |
| 6903 | 2:8149483 | 6907 | 2:8268124 | 6909 | 2:8273877 | 6925 | 2:8874962 | 6926 | 2:8891833 |
| 6948 | 2:9583773 | 6949 | 2:9583875 | 6957 | 2:9791102 | 6960 | 2:9831264 | 6985 | 2:10595947 |
| 6986 | 2:10596946 | 6987 | 2:10597759 | 6988 | 2:10603551 | 7032 | 2:12051677 | 7037 | 2:12305668 |
| 7043 | 2:12489693 | 7058 | 2:13087374 | 7071 | 2:13360171 | 7072 | 2:13382039 | 7082 | 2:13695930 |
| 7084 | 2:13709033 | 7086 | 2:13779018 | 7088 | 2:13797995 | 7090 | 2:13822009 | 7091 | 2:13823524 |
| 7092 | 2:13862800 | 7102 | 2:14216227 | 7127 | 2:14784977 | 7139 | 2:15119742 | 7147 | 2:15329884 |
| 7157 | 2:15549082 | 7162 | 2:15690371 | 7163 | 2:15693009 | 7164 | 2:15696748 | 7168 | 2:15821266 |
| 7173 | 2:15967374 | 7190 | 2:16339174 | 7207 | 2:16790984 | 7211 | 2:16932422 | 7217 | 2:17092308 |
| 7223 | 2:17311769 | 7236 | 2:17583628 | 7241 | 2:17659987 | 7242 | 2:17735524 | 7246 | 2:17833404 |
| 7250 | 2:17967333 | 7254 | 2:18038068 | 7260 | 2:18147142 | 7261 | 2:18165858 | 7263 | 2:18229834 |
| 7264 | 2:18229859 | 7266 | 2:18241132 | 7268 | 2:18244789 | 7270 | 2:18253097 | 7271 | 2:18254488 |
| 7273 | 2:18262337 | 7275 | 2:18270755 | 7276 | 2:18271356 | 7277 | 2:18271358 | 7278 | 2:18271418 |
| 7279 | 2:18272565 | 7280 | 2:18272965 | 7281 | 2:18275874 | 7282 | 2:18277584 | 7283 | 2:18277939 |
| 7284 | 2:18280757 | 7285 | 2:18280781 | 7286 | 2:18282254 | 7287 | 2:18282552 | 7288 | 2:18290509 |
| 7289 | 2:18291940 | 7290 | 2:18307699 | 7291 | 2:18323628 | 7293 | 2:18378637 | 7297 | 2:18502293 |
| 7304 | 2:18586886 | 7305 | 2:18595133 | 7306 | 2:18605578 | 7310 | 2:18642606 | 7314 | 2:18778867 |
| 7316 | 2:18866785 | 7317 | 2:18869734 | 7325 | 2:18981323 | 7326 | 2:18986957 | 7330 | 2:19045985 |
| 7335 | 2:19136987 | 7346 | 2:19340327 | 7352 | 2:19534377 | 7355 | 2:19593435 | 7356 | 2:19626883 |
| 7357 | 2:19648311 | 7358 | 2:19648602 | 7368 | 2:19897160 | 7369 | 2:19918403 | 7378 | 2:20155806 |
| 7380 | 2:20264485 | 7383 | 2:20344434 | 7387 | 2:20427608 | 7394 | 2:20674260 | 7401 | 2:20845761 |
| 7403 | 2:20861585 | 7407 | 2:21064906 | 7421 | 2:21401355 | 7422 | 2:21405561 | 7428 | 2:21553608 |
| 7438 | 2:21688312 | 7443 | 2:21849771 | 7444 | 2:21867557 | 7445 | 2:21867592 | 7447 | 2:21914587 |
| 7449 | 2:21967376 | 7451 | 2:22019700 | 7452 | 2:22051754 | 7458 | 2:22154085 | 7466 | 2:22396788 |
| 7472 | 2:22599197 | 7474 | 2:22608474 | 7479 | 2:22767655 | 7480 | 2:22787231 | 7485 | 2:23032717 |
| 7489 | 2:23063150 | 7496 | 2:23295824 | 7497 | 2:23312807 | 7502 | 2:23459486 | 7504 | 2:23476574 |
| 7505 | 2:23481265 | 7506 | 2:23481554 | 7507 | 2:23481572 | 7509 | 2:23487782 | 7510 | 2:23501169 |
| 7511 | 2:23537225 | 7512 | 2:23557793 | 7513 | 2:23558570 | 7520 | 2:23643567 | 7522 | 2:23666640 |
| 7523 | 2:23676088 | 7525 | 2:23691155 | 7526 | 2:23696931 | 7527 | 2:23699667 | 7534 | 2:23962288 |
| 7536 | 2:23971606 | 7545 | 2:24172853 | 7546 | 2:24176608 | 7552 | 2:24299477 | 7554 | 2:24320827 |
| 7555 | 2:24320845 | 7575 | 2:24669527 | 7586 | 2:24895083 | 7591 | 2:25095591 | 7595 | 2:25243017 |
| 7597 | 2:25329638 | 7600 | 2:25433245 | 7601 | 2:25441115 | 7602 | 2:25443814 | 7606 | 2:25523128 |
| 7607 | 2:25531340 | 7608 | 2:25539975 | 7609 | 2:25556600 | 7617 | 2:25649108 | 7620 | 2:25710237 |
| 7658 | 2:26534630 | 7667 | 2:26760780 | 7669 | 2:26762215 | 7670 | 2:26771611 | 7671 | 2:26774438 |
| 7674 | 2:26851479 | 7691 | 2:27096366 | 7698 | 2:27312219 | 7702 | 2:27432834 | 7727 | 2:27915981 |
| 7736 | 2:28189986 | 7738 | 2:28287714 | 7739 | 2:28287763 | 7740 | 2:28287812 | 7741 | 2:28287836 |
| 7746 | 2:28373427 | 7749 | 2:28418211 | 7764 | 2:28631973 | 7780 | 2:28937935 | 7813 | 2:29825101 |
| 7817 | 2:29924141 | 7825 | 2:30218788 | 7826 | 2:30225126 | 7827 | 2:30235162 | 7828 | 2:30235271 |
| 7837 | 2:30489470 | 7840 | 2:30505625 | 7847 | 2:30620848 | 7850 | 2:30714513 | 7852 | 2:30729817 |
| 7867 | 2:31118343 | 7868 | 2:31118369 | 7869 | 2:31121656 | 7887 | 2:31621372 | 7905 | 2:32163209 |
| 7921 | 2:32683358 | 7923 | 2:32754895 | 7930 | 2:33004780 | 7937 | 2:33199953 | 7948 | 2:33575101 |
| 7949 | 2:33575122 | 7950 | 2:33654382 | 7952 | 2:33671969 | 7953 | 2:33671986 | 7954 | 2:33694528 |
| 7959 | 2:33883065 | 7962 | 2:33960798 | 7978 | 2:34316478 | 7981 | 2:34353723 | 7986 | 2:34439099 |
| 7989 | 2:34507044 | 8027 | 2:35881554 | 8035 | 2:36123841 | 8036 | 2:36138834 | 8037 | 2:36147200 |
| 8051 | 2:36510607 | 8055 | 2:36554095 | 8056 | 2:36613946 | 8057 | 2:36621379 | 8060 | 2:36742166 |
| 8075 | 2:37300847 | 8101 | 2:38053061 | 8107 | 2:38237985 | 8108 | 2:38238121 | 8109 | 2:38238372 |
| 8110 | 2:38238415 | 8111 | 2:38238441 | 8112 | 2:38238520 | 8113 | 2:38238541 | 8117 | 2:38310384 |
| 8121 | 2:38442344 | 8123 | 2:38465755 | 8130 | 2:38600295 | 8131 | 2:38634299 | 8136 | 2:38811572 |
| 8163 | 2:39460697 | 8203 | 2:40835843 | 8205 | 2:40867544 | 8206 | 2:40867584 | 8207 | 2:40867585 |
| 8208 | 2:40867624 | 8209 | 2:40867647 | 8210 | 2:40867669 | 8211 | 2:40867710 | 8212 | 2:40867713 |
| 8215 | 2:41050502 | 8216 | 2:41050562 | 8217 | 2:41050564 | 8218 | 2:41050613 | 8226 | 2:41315483 |
| 8227 | 2:41315504 | 8245 | 2:41559277 | 8253 | 2:41775609 | 8254 | 2:41797511 | 8287 | 2:42520826 |
| 8288 | 2:42535114 | 8307 | 2:43006124 | 8321 | 2:43491727 | 8323 | 2:43580317 | 8324 | 2:43631101 |
| 8328 | 2:43711459 | 8332 | 2:43813071 | 8341 | 2:44152000 | 8343 | 2:44193529 | 8350 | 2:44379204 |
| 8351 | 2:44393093 | 8352 | 2:44393964 | 8353 | 2:44403432 | 8356 | 2:44515259 | 8357 | 2:44545955 |
| 8358 | 2:44569313 | 8360 | 2:44615292 | 8361 | 2:44621241 | 8367 | 2:44858072 | 8384 | 2:45372211 |
| 8388 | 2:45474859 | 8412 | 2:45983105 | 8415 | 2:46033456 | 8420 | 2:46147458 | 8428 | 2:46391874 |
| 8434 | 2:46437983 | 8436 | 2:46446081 | 8440 | 2:46534111 | 8446 | 2:46705643 | 8464 | 2:47013353 |
| 8467 | 2:47100164 | 8481 | 2:47433064 | 8486 | 2:47596275 | 8491 | 2:47706783 | 8509 | 2:48215370 |
| 8522 | 2:48603698 | 8523 | 2:48604345 | 8526 | 2:48755071 | 8528 | 2:48783617 | 8531 | 2:48830819 |
| 8534 | 2:48894232 | 8539 | 2:48987003 | 8544 | 2:49067927 | 8555 | 2:49577101 | 8571 | 2:50088797 |
| 8590 | 2:50645993 | 8595 | 2:50810980 | 8605 | 2:51033258 | 8615 | 2:51338525 | 8626 | 2:51770242 |
| 8628 | 2:51813793 | 8649 | 2:52303945 | 8655 | 2:52440698 | 8660 | 2:52515449 | 8671 | 2:52834508 |
| 8675 | 2:52903006 | 8676 | 2:52906421 | 8677 | 2:52909824 | 8681 | 2:52959066 | 8690 | 2:53226738 |
| 8735 | 2:54491736 | 8753 | 2:54901841 | 8756 | 2:54993038 | 8757 | 2:54993081 | 8758 | 2:54993084 |
| 8759 | 2:54993089 | 8761 | 2:55019715 | 8769 | 2:55150342 | 8770 | 2:55235366 | 8800 | 2:56234809 |
| 8802 | 2:56280951 | 8804 | 2:56314003 | 8809 | 2:56469340 | 8816 | 2:56646269 | 8838 | 2:57404778 |
| 8839 | 2:57430549 | 8849 | 2:57666386 | 8851 | 2:57700250 | 8858 | 2:57837920 | 8888 | 2:58761910 |
| 8890 | 2:58778117 | 8892 | 2:58793131 | 8897 | 2:58929038 | 8899 | 2:58940947 | 8901 | 2:58993535 |
| 8903 | 2:59042027 | 8904 | 2:59079693 | 8908 | 2:59202049 | 8909 | 2:59212187 | 8923 | 2:59601537 |
| 8931 | 2:59858246 | 8932 | 2:59858284 | 8933 | 2:59858319 | 8934 | 2:59858339 | 8949 | 2:60150792 |
| 8967 | 2:60751699 | 8978 | 2:61108555 | 8980 | 2:61144456 | 8981 | 2:61170888 | 8982 | 2:61172063 |
| 8985 | 2:61252370 | 8992 | 2:61343961 | 8994 | 2:61449061 | 8995 | 2:61490160 | 8996 | 2:61490261 |
| 8998 | 2:61494582 | 9000 | 2:61535058 | 9001 | 2:61535147 | 9002 | 2:61535149 | 9003 | 2:61535213 |
| 9013 | 2:61843883 | 9020 | 2:62046115 | 9024 | 2:62302155 | 9046 | 2:62786824 | 9051 | 2:62910878 |
| 9052 | 2:62944872 | 9053 | 2:62944925 | 9055 | 2:63002162 | 9065 | 2:63241642 | 9070 | 2:63325322 |
| 9078 | 2:63563631 | 9081 | 2:63616590 | 9090 | 2:63926681 | 9124 | 2:64713810 | 9129 | 2:64820071 |
| 9148 | 2:65218951 | 9164 | 2:65726104 | 9221 | 2:67528810 | 9226 | 2:67669505 | 9236 | 2:68034573 |
| 9267 | 2:69100324 | 9283 | 2:69720593 | 9294 | 2:70008307 | 9303 | 2:70294251 | 9304 | 2:70313073 |
| 9310 | 2:70440178 | 9324 | 2:70768201 | 9329 | 2:70931242 | 9334 | 2:71041850 | 9335 | 2:71042132 |
| 9336 | 2:71042190 | 9337 | 2:71044225 | 9338 | 2:71095900 | 9341 | 2:71203819 | 9342 | 2:71205559 |
| 9349 | 2:71337903 | 9357 | 2:71501065 | 9366 | 2:71912340 | 9375 | 2:72314326 | 9392 | 2:72649795 |
| 9393 | 2:72669915 | 9394 | 2:72669943 | 9403 | 2:72918814 | 9404 | 2:72977278 | 9405 | 2:72995902 |
| 9407 | 2:73090036 | 9413 | 2:73157226 | 9414 | 2:73160964 | 9415 | 2:73161983 | 9420 | 2:73383708 |
| 9422 | 2:73398069 | 9424 | 2:73477830 | 9434 | 2:73757540 | 9437 | 2:73811343 | 9446 | 2:73945664 |
| 9449 | 2:74083815 | 9468 | 2:74707536 | 9469 | 2:74707559 | 9470 | 2:74707612 | 9471 | 2:74707658 |
| 9472 | 2:74707710 | 9482 | 2:75029605 | 9485 | 2:75173951 | 9493 | 2:75482167 | 9494 | 2:75483513 |
| 9501 | 2:75692826 | 9507 | 2:75820235 | 9508 | 2:75834257 | 9517 | 2:76002206 | 9519 | 2:76048349 |
| 9527 | 2:76266636 | 9537 | 2:76475875 | 9539 | 2:76503911 | 9556 | 2:77018992 | 9561 | 2:77216172 |
| 9563 | 2:77216223 | 9570 | 2:77393386 | 9571 | 2:77423386 | 9575 | 2:77530544 | 9579 | 2:77676860 |
| 9580 | 2:77680269 | 9581 | 2:77690134 | 9583 | 2:77796311 | 9590 | 2:77930065 | 9591 | 2:77960137 |
| 9592 | 2:77960148 | 9593 | 2:77960205 | 9594 | 2:77960221 | 9595 | 2:78058933 | 9599 | 2:78093605 |
| 9608 | 2:78475004 | 9610 | 2:78576254 | 9621 | 2:78787744 | 9622 | 2:78787785 | 9623 | 2:78793670 |
| 9625 | 2:78828241 | 9627 | 2:78856439 | 9631 | 2:78919311 | 9632 | 2:78922277 | 9634 | 2:78933501 |
| 9635 | 2:78933506 | 9636 | 2:78940906 | 9637 | 2:78942899 | 9640 | 2:79034805 | 9643 | 2:79043516 |
| 9665 | 2:79805247 | 9673 | 2:79959399 | 9683 | 2:80095415 | 9686 | 2:80170719 | 9762 | 2:83630529 |
| 9763 | 2:83648977 | 9764 | 2:83654198 | 9765 | 2:83657997 | 9766 | 2:83660404 | 9767 | 2:83664117 |
| 9768 | 2:83664826 | 9769 | 2:83665834 | 9770 | 2:83667393 | 9771 | 2:83669899 | 9772 | 2:83671239 |
| 9774 | 2:83694268 | 9775 | 2:83694872 | 9776 | 2:83696501 | 9777 | 2:83697095 | 9778 | 2:83701382 |
| 9779 | 2:83703221 | 9780 | 2:83710452 | 9796 | 2:84314672 | 9801 | 2:84413080 | 9806 | 2:84544851 |
| 9807 | 2:84554472 | 9808 | 2:84556946 | 9809 | 2:84558765 | 9810 | 2:84558813 | 9811 | 2:84579866 |
| 9812 | 2:84579884 | 9814 | 2:84594590 | 9815 | 2:84594621 | 9816 | 2:84631977 | 9821 | 2:84747533 |
| 9826 | 2:84895919 | 9829 | 2:84986688 | 9841 | 2:85268807 | 9842 | 2:85302489 | 9857 | 2:85748841 |
| 9868 | 2:85921857 | 9870 | 2:85984791 | 9871 | 2:86008072 | 9876 | 2:86150518 | 9881 | 2:86310570 |
| 9882 | 2:86387502 | 9886 | 2:86419348 | 9889 | 2:86436688 | 9891 | 2:86469285 | 9894 | 2:86509868 |
| 9961 | 2:88510673 | 9965 | 2:88623312 | 9970 | 2:88707441 | 9971 | 2:88719162 | 9973 | 2:88746032 |
| 9975 | 2:88782810 | 9977 | 2:88859157 | 9983 | 2:89070767 | 9984 | 2:89076240 | 9985 | 2:89084301 |
| 9986 | 2:89086388 | 9987 | 2:89095014 | 9988 | 2:89100826 | 9989 | 2:89104328 | 9990 | 2:89109055 |
| 9991 | 2:89114149 | 9992 | 2:89117889 | 9993 | 2:89119778 | 9994 | 2:89122696 | 9995 | 2:89126698 |
| 9996 | 2:89128778 | 9997 | 2:89129756 | 9998 | 2:89130672 | 9999 | 2:89131642 | 10000 | 2:89132131 |
| 10001 | 2:89132635 | 10002 | 2:89133746 | 10003 | 2:89136358 | 10004 | 2:89136948 | 10005 | 2:89143985 |
| 10006 | 2:89150132 | 10007 | 2:89157341 | 10008 | 2:89161844 | 10009 | 2:89164769 | 10010 | 2:89172177 |
| 10011 | 2:89183562 | 10012 | 2:89188097 | 10013 | 2:89192815 | 10014 | 2:89196778 | 10015 | 2:89206007 |
| 10016 | 2:89210274 | 10017 | 2:89220562 | 10021 | 2:89293059 | 10022 | 2:89329459 | 10023 | 2:89330715 |
| 10024 | 2:89332510 | 10025 | 2:89333883 | 10026 | 2:89335072 | 10028 | 2:89337942 | 10031 | 2:89377233 |
| 10034 | 2:89445178 | 10049 | 2:89808466 | 10050 | 2:89813015 | 10052 | 2:89841096 | 10054 | 2:89876885 |
| 10055 | 2:89878690 | 10057 | 2:89908253 | 10059 | 2:89916678 | 10060 | 2:89948655 | 10061 | 2:89952700 |
| 10062 | 2:89952808 | 10063 | 2:89953249 | 10064 | 2:89964009 | 10065 | 2:89964459 | 10066 | 2:89964908 |
| 10100 | 2:91309327 | 10101 | 2:91319564 | 10123 | 2:92012717 | 10131 | 2:92095096 | 10132 | 2:92109122 |
| 10142 | 2:92406522 | 10143 | 2:92416012 | 10146 | 2:92501151 | 10148 | 2:92504237 | 10154 | 2:92593249 |
| 10157 | 2:92677123 | 10158 | 2:92688749 | 10163 | 2:92792925 | 10165 | 2:92800370 | 10167 | 2:92859552 |
| 10168 | 2:92875951 | 10171 | 2:93007619 | 10197 | 2:93432778 | 10217 | 2:93888418 | 10231 | 2:94155809 |
| 10233 | 2:94177314 | 10234 | 2:94238285 | 10238 | 2:94284858 | 10242 | 2:94408041 | 10244 | 2:94532812 |
| 10250 | 2:94662652 | 10263 | 2:94943212 | 10269 | 2:95104701 | 10273 | 2:95191751 | 10276 | 2:95262942 |
| 10277 | 2:95265410 | 10297 | 2:95781931 | 10298 | 2:95788314 | 10299 | 2:95797570 | 10304 | 2:95957388 |
| 10307 | 2:96027434 | 10312 | 2:96139223 | 10313 | 2:96162249 | 10321 | 2:96318089 | 10324 | 2:96352046 |
| 10330 | 2:96463623 | 10332 | 2:96505449 | 10333 | 2:96512869 | 10334 | 2:96514006 | 10335 | 2:96514035 |
| 10336 | 2:96514066 | 10337 | 2:96514071 | 10339 | 2:96516949 | 10340 | 2:96517000 | 10341 | 2:96518404 |
| 10342 | 2:96519726 | 10343 | 2:96519764 | 10344 | 2:96519823 | 10345 | 2:96522707 | 10346 | 2:96522837 |
| 10352 | 2:96721652 | 10373 | 2:97569993 | 10393 | 2:98098074 | 10394 | 2:98098574 | 10397 | 2:98189476 |
| 10411 | 2:98434332 | 10435 | 2:99027387 | 10443 | 2:99309195 | 10444 | 2:99310709 | 10477 | 2:100060201 |
| 10486 | 2:100359058 | 10488 | 2:100436598 | 10489 | 2:100436640 | 10490 | 2:100507665 | 10491 | 2:100527030 |
| 10508 | 2:100969194 | 10512 | 2:101142510 | 10515 | 2:101176628 | 10516 | 2:101267741 | 10522 | 2:101470754 |
| 10524 | 2:101503694 | 10526 | 2:101544902 | 10529 | 2:101696463 | 10540 | 2:102030097 | 10541 | 2:102031180 |
| 10567 | 2:102763970 | 10569 | 2:102854361 | 10570 | 2:102861535 | 10574 | 2:103021453 | 10575 | 2:103022409 |
| 10577 | 2:103050047 | 10578 | 2:103087349 | 10588 | 2:103396204 | 10589 | 2:103402752 | 10592 | 2:103445587 |
| 10595 | 2:103517827 | 10598 | 2:103593199 | 10610 | 2:103884686 | 10615 | 2:104008673 | 10622 | 2:104115856 |
| 10628 | 2:104308034 | 10635 | 2:104472870 | 10641 | 2:104678498 | 10643 | 2:104790859 | 10644 | 2:104814826 |
| 10646 | 2:104850637 | 10647 | 2:104882698 | 10660 | 2:105042627 | 10669 | 2:105160301 | 10679 | 2:105493554 |
| 10682 | 2:105532073 | 10684 | 2:105558282 | 10685 | 2:105589302 | 10689 | 2:105726409 | 10697 | 2:105850938 |
| 10698 | 2:105883087 | 10702 | 2:105943923 | 10704 | 2:106005198 | 10705 | 2:106040867 | 10708 | 2:106093074 |
| 10709 | 2:106134401 | 10716 | 2:106279517 | 10718 | 2:106396433 | 10719 | 2:106446238 | 10720 | 2:106455619 |
| 10722 | 2:106582615 | 10724 | 2:106599852 | 10725 | 2:106602909 | 10726 | 2:106605195 | 10727 | 2:106609300 |
| 10728 | 2:106609491 | 10729 | 2:106615459 | 10730 | 2:106624131 | 10731 | 2:106632743 | 10732 | 2:106635677 |
| 10740 | 2:106788337 | 10748 | 2:106865812 | 10750 | 2:106960370 | 10752 | 2:107059040 | 10753 | 2:107076804 |
| 10766 | 2:107558581 | 10768 | 2:107560640 | 10769 | 2:107560655 | 10773 | 2:107620491 | 10774 | 2:107628799 |
| 10777 | 2:107692575 | 10780 | 2:107718206 | 10793 | 2:107772997 | 10796 | 2:107824497 | 10797 | 2:107827321 |
| 10799 | 2:107881110 | 10808 | 2:108144100 | 10810 | 2:108165798 | 10820 | 2:108449215 | 10827 | 2:108649909 |
| 10835 | 2:108855822 | 10838 | 2:108918709 | 10839 | 2:108932264 | 10841 | 2:108944061 | 10850 | 2:109135684 |
| 10861 | 2:109403406 | 10862 | 2:109415989 | 10867 | 2:109507997 | 10868 | 2:109525518 | 10871 | 2:109605505 |
| 10874 | 2:109627420 | 10878 | 2:109678083 | 10896 | 2:110207989 | 10909 | 2:110556883 | 10919 | 2:111069391 |
| 10923 | 2:111211200 | 10924 | 2:111216842 | 10931 | 2:111473451 | 10946 | 2:111903128 | 10950 | 2:112002693 |
| 10951 | 2:112064672 | 10952 | 2:112068968 | 10963 | 2:112309161 | 10982 | 2:112824303 | 10985 | 2:112865726 |
| 10988 | 2:112884446 | 10996 | 2:112999914 | 10998 | 2:113039809 | 11000 | 2:113071684 | 11004 | 2:113217166 |
| 11005 | 2:113217179 | 11006 | 2:113217208 | 11007 | 2:113217254 | 11033 | 2:113775291 | 11040 | 2:113927720 |
| 11052 | 2:114312086 | 11056 | 2:114441340 | 11057 | 2:114511997 | 11070 | 2:114795811 | 11072 | 2:114857637 |
| 11087 | 2:115277541 | 11088 | 2:115279258 | 11092 | 2:115316715 | 11096 | 2:115405473 | 11097 | 2:115455721 |
| 11106 | 2:115621193 | 11108 | 2:115625826 | 11125 | 2:115953383 | 11155 | 2:116733465 | 11161 | 2:116888348 |
| 11167 | 2:117033411 | 11168 | 2:117047832 | 11169 | 2:117079799 | 11172 | 2:117133057 | 11178 | 2:117240992 |
| 11187 | 2:117466579 | 11195 | 2:117945652 | 11200 | 2:118099830 | 11207 | 2:118201421 | 11220 | 2:118509097 |
| 11222 | 2:118554241 | 11239 | 2:119145772 | 11240 | 2:119196348 | 11241 | 2:119198191 | 11242 | 2:119199613 |
| 11244 | 2:119244130 | 11245 | 2:119264906 | 11248 | 2:119291176 | 11252 | 2:119322052 | 11257 | 2:119465613 |
| 11260 | 2:119511934 | 11261 | 2:119512418 | 11262 | 2:119522741 | 11263 | 2:119526528 | 11264 | 2:119533194 |
| 11267 | 2:119548536 | 11269 | 2:119562713 | 11270 | 2:119564586 | 11271 | 2:119565180 | 11272 | 2:119566000 |
| 11273 | 2:119576736 | 11277 | 2:119825482 | 11278 | 2:119862058 | 11290 | 2:120178985 | 11292 | 2:120195958 |
| 11293 | 2:120199797 | 11308 | 2:120568141 | 11310 | 2:120671914 | 11319 | 2:120900260 | 11322 | 2:120932791 |
| 11331 | 2:121151913 | 11333 | 2:121188721 | 11352 | 2:121755672 | 11358 | 2:121893423 | 11359 | 2:121894763 |
| 11375 | 2:122580635 | 11382 | 2:122800513 | 11385 | 2:122878392 | 11393 | 2:123070777 | 11394 | 2:123070905 |
| 11395 | 2:123070944 | 11396 | 2:123100345 | 11397 | 2:123100370 | 11414 | 2:123539540 | 11415 | 2:123539541 |
| 11417 | 2:123562349 | 11419 | 2:123590694 | 11420 | 2:123590830 | 11421 | 2:123610591 | 11427 | 2:123692694 |
| 11447 | 2:124332744 | 11453 | 2:124600152 | 11458 | 2:124710998 | 11462 | 2:124913543 | 11470 | 2:125166728 |
| 11476 | 2:125250396 | 11479 | 2:125277549 | 11480 | 2:125286027 | 11481 | 2:125349483 | 11490 | 2:125625209 |
| 11492 | 2:125653640 | 11493 | 2:125653952 | 11496 | 2:125742333 | 11499 | 2:125810128 | 11502 | 2:125931055 |
| 11503 | 2:125952736 | 11508 | 2:126114829 | 11511 | 2:126173762 | 11515 | 2:126267198 | 11516 | 2:126267881 |
| 11517 | 2:126273576 | 11518 | 2:126275683 | 11519 | 2:126286517 | 11522 | 2:126366193 | 11523 | 2:126367550 |
| 11525 | 2:126372344 | 11526 | 2:126380236 | 11529 | 2:126489135 | 11575 | 2:127003970 | 11576 | 2:127004702 |
| 11583 | 2:127018411 | 11587 | 2:127027686 | 11589 | 2:127038523 | 11606 | 2:127110467 | 11613 | 2:127238953 |
| 11623 | 2:127397394 | 11629 | 2:127525976 | 11634 | 2:127633593 | 11636 | 2:127635918 | 11637 | 2:127639135 |
| 11654 | 2:128049090 | 11656 | 2:128099391 | 11672 | 2:128508443 | 11673 | 2:128508479 | 11677 | 2:128603208 |
| 11683 | 2:128718168 | 11688 | 2:128782288 | 11691 | 2:128791601 | 11692 | 2:128804408 | 11695 | 2:128916878 |
| 11701 | 2:129068395 | 11702 | 2:129073892 | 11704 | 2:129084385 | 11705 | 2:129095315 | 11706 | 2:129098350 |
| 11707 | 2:129101237 | 11719 | 2:129414787 | 11721 | 2:129451897 | 11722 | 2:129453738 | 11723 | 2:129463584 |
| 11724 | 2:129475673 | 11725 | 2:129514375 | 11735 | 2:129823411 | 11740 | 2:129952968 | 11750 | 2:130179342 |
| 11751 | 2:130179458 | 11753 | 2:130297720 | 11759 | 2:130405723 | 11761 | 2:130416105 | 11771 | 2:130763239 |
| 11773 | 2:130778311 | 11775 | 2:130786713 | 11777 | 2:130887344 | 11779 | 2:130922023 | 11780 | 2:130922555 |
| 11781 | 2:130924042 | 11786 | 2:131056297 | 11788 | 2:131116831 | 11792 | 2:131182393 | 11793 | 2:131183245 |
| 11799 | 2:131239243 | 11810 | 2:131495314 | 11812 | 2:131522458 | 11818 | 2:131759255 | 11827 | 2:131997425 |
| 11828 | 2:132032080 | 11829 | 2:132052956 | 11836 | 2:132152838 | 11850 | 2:132304236 | 11856 | 2:132453956 |
| 11862 | 2:132630596 | 11873 | 2:132828927 | 11874 | 2:132829391 | 11883 | 2:133050393 | 11884 | 2:133069759 |
| 11886 | 2:133077482 | 11888 | 2:133094549 | 11889 | 2:133095512 | 11892 | 2:133207446 | 11897 | 2:133305481 |
| 11901 | 2:133364775 | 11905 | 2:133421233 | 11908 | 2:133536795 | 11910 | 2:133542411 | 11915 | 2:133623325 |
| 11923 | 2:133694191 | 11927 | 2:133778125 | 11928 | 2:133778616 | 11929 | 2:133785516 | 11931 | 2:133786535 |
| 11932 | 2:133787224 | 11933 | 2:133787956 | 11934 | 2:133787982 | 11935 | 2:133788783 | 11957 | 2:134106505 |
| 11963 | 2:134189401 | 11964 | 2:134189458 | 11965 | 2:134190122 | 11966 | 2:134190219 | 11977 | 2:134376164 |
| 11979 | 2:134438884 | 11982 | 2:134480181 | 11983 | 2:134480249 | 11984 | 2:134480295 | 11985 | 2:134480304 |
| 11988 | 2:134508261 | 11997 | 2:134616684 | 11998 | 2:134616699 | 12008 | 2:134803486 | 12012 | 2:134834209 |
| 12013 | 2:134843772 | 12019 | 2:134917533 | 12020 | 2:134917563 | 12021 | 2:134917597 | 12022 | 2:134917643 |
| 12023 | 2:134917658 | 12031 | 2:135091858 | 12033 | 2:135100100 | 12035 | 2:135114924 | 12038 | 2:135166182 |
| 12039 | 2:135171128 | 12042 | 2:135233030 | 12043 | 2:135235158 | 12048 | 2:135319271 | 12054 | 2:135454017 |
| 12058 | 2:135514495 | 12063 | 2:135598504 | 12064 | 2:135598610 | 12066 | 2:135604349 | 12069 | 2:135646413 |
| 12074 | 2:135709576 | 12076 | 2:135727874 | 12077 | 2:135727928 | 12079 | 2:135837161 | 12080 | 2:135837263 |
| 12082 | 2:135865945 | 12083 | 2:135887710 | 12084 | 2:135900620 | 12085 | 2:135900833 | 12086 | 2:135904261 |
| 12087 | 2:135908217 | 12091 | 2:136093517 | 12092 | 2:136093612 | 12104 | 3:197807 | 12127 | 3:746327 |
| 12135 | 3:867632 | 12139 | 3:923989 | 12140 | 3:924352 | 12142 | 3:941811 | 12146 | 3:969297 |
| 12155 | 3:1114174 | 12162 | 3:1183922 | 12181 | 3:1651460 | 12184 | 3:1672587 | 12188 | 3:1729118 |
| 12195 | 3:1939081 | 12198 | 3:1965533 | 12199 | 3:1967307 | 12204 | 3:2023241 | 12206 | 3:2062805 |
| 12208 | 3:2064509 | 12211 | 3:2089682 | 12212 | 3:2098556 | 12213 | 3:2104449 | 12235 | 3:2740113 |
| 12243 | 3:2986445 | 12245 | 3:2993307 | 12250 | 3:3052396 | 12283 | 3:3863731 | 12305 | 3:4250424 |
| 12306 | 3:4264560 | 12307 | 3:4280470 | 12308 | 3:4285570 | 12310 | 3:4298685 | 12312 | 3:4313521 |
| 12314 | 3:4363949 | 12316 | 3:4398654 | 12332 | 3:4566642 | 12335 | 3:4593309 | 12338 | 3:4692667 |
| 12344 | 3:4732630 | 12345 | 3:4732659 | 12354 | 3:4874925 | 12355 | 3:4874954 | 12373 | 3:5268053 |
| 12377 | 3:5309746 | 12383 | 3:5436199 | 12384 | 3:5462355 | 12408 | 3:5998482 | 12422 | 3:6207469 |
| 12424 | 3:6282733 | 12425 | 3:6293053 | 12432 | 3:6517775 | 12443 | 3:6741890 | 12444 | 3:6771925 |
| 12445 | 3:6771928 | 12446 | 3:6771969 | 12447 | 3:6772038 | 12469 | 3:7060982 | 12470 | 3:7106174 |
| 12478 | 3:7235310 | 12479 | 3:7238069 | 12481 | 3:7255164 | 12483 | 3:7357130 | 12486 | 3:7383219 |
| 12492 | 3:7455499 | 12506 | 3:7653471 | 12511 | 3:7783911 | 12512 | 3:7822809 | 12514 | 3:7838838 |
| 12522 | 3:7931685 | 12523 | 3:7931828 | 12524 | 3:7931887 | 12528 | 3:7961056 | 12537 | 3:8147516 |
| 12538 | 3:8163086 | 12539 | 3:8174824 | 12540 | 3:8183497 | 12541 | 3:8184872 | 12542 | 3:8186958 |
| 12543 | 3:8193625 | 12544 | 3:8194122 | 12545 | 3:8208066 | 12551 | 3:8277038 | 12552 | 3:8300299 |
| 12555 | 3:8337519 | 12570 | 3:8661008 | 12584 | 3:9427220 | 12587 | 3:9485193 | 12588 | 3:9485906 |
| 12590 | 3:9513246 | 12594 | 3:9564986 | 12595 | 3:9591739 | 12598 | 3:9636755 | 12605 | 3:9764080 |
| 12606 | 3:9832615 | 12608 | 3:9847485 | 12609 | 3:9858051 | 12612 | 3:9863664 | 12622 | 3:10118782 |
| 12623 | 3:10118907 | 12624 | 3:10119257 | 12625 | 3:10119319 | 12626 | 3:10122375 | 12627 | 3:10122970 |
| 12628 | 3:10123769 | 12629 | 3:10128298 | 12630 | 3:10132847 | 12631 | 3:10133516 | 12632 | 3:10134377 |
| 12633 | 3:10136175 | 12634 | 3:10137707 | 12635 | 3:10140467 | 12636 | 3:10141169 | 12637 | 3:10156326 |
| 12638 | 3:10159182 | 12639 | 3:10177781 | 12641 | 3:10178026 | 12642 | 3:10178076 | 12643 | 3:10178165 |
| 12644 | 3:10178365 | 12645 | 3:10178537 | 12652 | 3:10343580 | 12656 | 3:10373626 | 12662 | 3:10458829 |
| 12663 | 3:10458901 | 12664 | 3:10458904 | 12665 | 3:10458919 | 12666 | 3:10458930 | 12667 | 3:10458938 |
| 12668 | 3:10459027 | 12669 | 3:10459091 | 12670 | 3:10459139 | 12671 | 3:10459202 | 12672 | 3:10459244 |
| 12673 | 3:10459247 | 12674 | 3:10459248 | 12675 | 3:10459299 | 12676 | 3:10459407 | 12677 | 3:10459424 |
| 12679 | 3:10497816 | 12680 | 3:10497887 | 12681 | 3:10497945 | 12682 | 3:10497948 | 12683 | 3:10497963 |
| 12684 | 3:10498011 | 12686 | 3:10519036 | 12688 | 3:10519145 | 12689 | 3:10526199 | 12690 | 3:10526562 |
| 12691 | 3:10526604 | 12692 | 3:10527562 | 12693 | 3:10527602 | 12694 | 3:10527675 | 12700 | 3:10725165 |
| 12703 | 3:10854110 | 12704 | 3:10854114 | 12705 | 3:10854133 | 12706 | 3:10854173 | 12707 | 3:10854726 |
| 12708 | 3:10854773 | 12710 | 3:10868768 | 12738 | 3:11492432 | 12739 | 3:11524924 | 12740 | 3:11525352 |
| 12741 | 3:11525876 | 12744 | 3:11607381 | 12746 | 3:11634350 | 12747 | 3:11634398 | 12748 | 3:11635619 |
| 12762 | 3:12154189 | 12786 | 3:12876946 | 12792 | 3:13018051 | 12799 | 3:13164663 | 12815 | 3:13606552 |
| 12818 | 3:13623598 | 12819 | 3:13623632 | 12820 | 3:13624757 | 12829 | 3:13851188 | 12839 | 3:13973650 |
| 12841 | 3:13997525 | 12844 | 3:14032792 | 12848 | 3:14146757 | 12849 | 3:14158851 | 12850 | 3:14172051 |
| 12851 | 3:14184603 | 12852 | 3:14185315 | 12853 | 3:14185422 | 12854 | 3:14188705 | 12857 | 3:14233203 |
| 12858 | 3:14239901 | 12859 | 3:14259325 | 12862 | 3:14312950 | 12867 | 3:14498473 | 12868 | 3:14500940 |
| 12870 | 3:14546904 | 12874 | 3:14640547 | 12877 | 3:14754130 | 12879 | 3:14774051 | 12883 | 3:14797864 |
| 12888 | 3:14853239 | 12891 | 3:14983274 | 12895 | 3:15110207 | 12897 | 3:15181630 | 12899 | 3:15254497 |
| 12904 | 3:15408885 | 12905 | 3:15410634 | 12906 | 3:15410653 | 12907 | 3:15410660 | 12911 | 3:15464742 |
| 12915 | 3:15536943 | 12917 | 3:15587630 | 12921 | 3:15632301 | 12923 | 3:15659384 | 12929 | 3:15702216 |
| 12932 | 3:15793894 | 12933 | 3:15794524 | 12945 | 3:15926790 | 12950 | 3:16002303 | 12955 | 3:16101556 |
| 12956 | 3:16101690 | 12957 | 3:16153064 | 12961 | 3:16228126 | 12964 | 3:16347204 | 12978 | 3:16700799 |
| 12979 | 3:16719574 | 12980 | 3:16719945 | 12981 | 3:16720763 | 12982 | 3:16720823 | 12983 | 3:16720842 |
| 12984 | 3:16721218 | 12985 | 3:16723559 | 12986 | 3:16725242 | 13003 | 3:17249309 | 13035 | 3:18290761 |
| 13058 | 3:18945520 | 13061 | 3:19021728 | 13063 | 3:19054577 | 13070 | 3:19211257 | 13071 | 3:19217706 |
| 13072 | 3:19217713 | 13073 | 3:19218362 | 13074 | 3:19225387 | 13075 | 3:19225406 | 13076 | 3:19225421 |
| 13077 | 3:19225424 | 13078 | 3:19225426 | 13079 | 3:19225509 | 13082 | 3:19277608 | 13089 | 3:19513415 |
| 13094 | 3:19685974 | 13097 | 3:19711385 | 13099 | 3:19775942 | 13101 | 3:19816181 | 13109 | 3:20143190 |
| 13110 | 3:20145668 | 13112 | 3:20160583 | 13116 | 3:20338589 | 13123 | 3:20549806 | 13125 | 3:20564672 |
| 13130 | 3:20805323 | 13132 | 3:20877656 | 13142 | 3:21424575 | 13168 | 3:22009877 | 13173 | 3:22059782 |
| 13178 | 3:22108825 | 13201 | 3:22592195 | 13206 | 3:22780511 | 13207 | 3:22808543 | 13209 | 3:22841288 |
| 13211 | 3:22869028 | 13214 | 3:22938448 | 13215 | 3:22963089 | 13219 | 3:23051090 | 13220 | 3:23087313 |
| 13225 | 3:23186080 | 13231 | 3:23299798 | 13233 | 3:23367141 | 13235 | 3:23401470 | 13242 | 3:23543571 |
| 13259 | 3:24023430 | 13260 | 3:24023439 | 13265 | 3:24145693 | 13266 | 3:24151362 | 13269 | 3:24196352 |
| 13270 | 3:24213490 | 13272 | 3:24238856 | 13281 | 3:24394654 | 13283 | 3:24424423 | 13301 | 3:24950077 |
| 13308 | 3:25150752 | 13310 | 3:25164615 | 13311 | 3:25170221 | 13312 | 3:25173303 | 13331 | 3:25597219 |
| 13332 | 3:25600844 | 13344 | 3:26002177 | 13346 | 3:26027856 | 13347 | 3:26036214 | 13348 | 3:26044741 |
| 13349 | 3:26046291 | 13359 | 3:26212407 | 13360 | 3:26212494 | 13361 | 3:26212691 | 13363 | 3:26256361 |
| 13371 | 3:26380532 | 13372 | 3:26391670 | 13377 | 3:26439980 | 13378 | 3:26469071 | 13379 | 3:26473706 |
| 13391 | 3:26893372 | 13395 | 3:26962431 | 13397 | 3:26979935 | 13399 | 3:27052772 | 13400 | 3:27057546 |
| 13401 | 3:27081349 | 13409 | 3:27180648 | 13413 | 3:27264556 | 13414 | 3:27294196 | 13417 | 3:27380069 |
| 13418 | 3:27382883 | 13419 | 3:27384412 | 13423 | 3:27481652 | 13452 | 3:27642990 | 13454 | 3:27663059 |
| 13458 | 3:27753499 | 13459 | 3:27756989 | 13463 | 3:27871144 | 13465 | 3:27916123 | 13468 | 3:27942850 |
| 13469 | 3:27942868 | 13472 | 3:27986412 | 13473 | 3:27990865 | 13476 | 3:28061404 | 13495 | 3:28713706 |
| 13509 | 3:29019861 | 13537 | 3:29488699 | 13545 | 3:29585223 | 13546 | 3:29592827 | 13547 | 3:29597761 |
| 13548 | 3:29601864 | 13550 | 3:29625965 | 13555 | 3:29681833 | 13558 | 3:29705190 | 13559 | 3:29705342 |
| 13560 | 3:29705512 | 13561 | 3:29706025 | 13562 | 3:29706295 | 13563 | 3:29706336 | 13568 | 3:29857727 |
| 13570 | 3:29915442 | 13571 | 3:29944912 | 13573 | 3:29954086 | 13575 | 3:29989198 | 13588 | 3:30363529 |
| 13607 | 3:30832758 | 13613 | 3:30890540 | 13619 | 3:31013256 | 13622 | 3:31065769 | 13627 | 3:31222103 |
| 13630 | 3:31343441 | 13645 | 3:31640573 | 13646 | 3:31689225 | 13652 | 3:31856497 | 13654 | 3:31864015 |
| 13655 | 3:31867053 | 13657 | 3:31896363 | 13664 | 3:32046129 | 13665 | 3:32047685 | 13666 | 3:32079966 |
| 13668 | 3:32103555 | 13671 | 3:32156925 | 13673 | 3:32164330 | 13676 | 3:32278723 | 13681 | 3:32512486 |
| 13699 | 3:32780141 | 13738 | 3:32985901 | 13743 | 3:33003020 | 13762 | 3:33254753 | 13780 | 3:33545907 |
| 13781 | 3:33549574 | 13784 | 3:33581206 | 13786 | 3:33623020 | 13787 | 3:33626853 | 13788 | 3:33628444 |
| 13789 | 3:33635163 | 13790 | 3:33641481 | 13791 | 3:33644276 | 13792 | 3:33647252 | 13793 | 3:33648187 |
| 13794 | 3:33652206 | 13795 | 3:33655047 | 13796 | 3:33667890 | 13797 | 3:33668793 | 13798 | 3:33673078 |
| 13799 | 3:33679729 | 13800 | 3:33680460 | 13806 | 3:33800750 | 13810 | 3:33917904 | 13812 | 3:33969180 |
| 13814 | 3:33975547 | 13819 | 3:34094670 | 13828 | 3:34304343 | 13832 | 3:34398092 | 13839 | 3:34520818 |
| 13840 | 3:34541029 | 13841 | 3:34544910 | 13844 | 3:34626494 | 13845 | 3:34639336 | 13846 | 3:34639367 |
| 13847 | 3:34639414 | 13848 | 3:34643371 | 13849 | 3:34644168 | 13850 | 3:34644227 | 13851 | 3:34644230 |
| 13852 | 3:34644292 | 13853 | 3:34644311 | 13854 | 3:34646033 | 13855 | 3:34648675 | 13856 | 3:34648858 |
| 13857 | 3:34654205 | 13858 | 3:34702016 | 13862 | 3:34749792 | 13865 | 3:34794868 | 13871 | 3:34932689 |
| 13872 | 3:34999359 | 13880 | 3:35139460 | 13881 | 3:35139484 | 13886 | 3:35172154 | 13889 | 3:35229235 |
| 13892 | 3:35308462 | 13902 | 3:35598523 | 13905 | 3:35702543 | 13906 | 3:35714205 | 13910 | 3:35853066 |
| 13914 | 3:35918054 | 13919 | 3:36000811 | 13923 | 3:36106918 | 13924 | 3:36139522 | 13930 | 3:36276403 |
| 13937 | 3:36323043 | 13940 | 3:36402532 | 13941 | 3:36414305 | 13950 | 3:36709252 | 13951 | 3:36709316 |
| 13958 | 3:36906114 | 13985 | 3:37656931 | 13986 | 3:37657062 | 13991 | 3:37904335 | 14029 | 3:39458322 |
| 14043 | 3:39836580 | 14050 | 3:40045551 | 14052 | 3:40104674 | 14057 | 3:40188225 | 14075 | 3:40698452 |
| 14081 | 3:41015843 | 14084 | 3:41043374 | 14088 | 3:41141556 | 14093 | 3:41367055 | 14097 | 3:41491123 |
| 14100 | 3:41527680 | 14101 | 3:41534229 | 14103 | 3:41589506 | 14104 | 3:41589571 | 14107 | 3:41614043 |
| 14113 | 3:41934611 | 14114 | 3:41939570 | 14115 | 3:41940253 | 14117 | 3:41944095 | 14118 | 3:41945371 |
| 14119 | 3:41946261 | 14124 | 3:42082768 | 14136 | 3:42496233 | 14137 | 3:42502863 | 14140 | 3:42614221 |
| 14141 | 3:42635738 | 14143 | 3:42650352 | 14144 | 3:42659935 | 14148 | 3:42725238 | 14152 | 3:42769882 |
| 14155 | 3:42840302 | 14156 | 3:42872218 | 14159 | 3:42900902 | 14160 | 3:42978663 | 14165 | 3:43002941 |
| 14167 | 3:43038774 | 14175 | 3:43325595 | 14176 | 3:43325725 | 14177 | 3:43334230 | 14182 | 3:43389979 |
| 14183 | 3:43397421 | 14185 | 3:43426451 | 14193 | 3:43680161 | 14194 | 3:43696024 | 14195 | 3:43698262 |
| 14196 | 3:43698948 | 14203 | 3:43873693 | 14206 | 3:44017413 | 14215 | 3:44305690 | 14226 | 3:44620511 |
| 14235 | 3:44902917 | 14238 | 3:44948682 | 14260 | 3:45663768 | 14298 | 3:46955918 | 14299 | 3:46978051 |
| 14309 | 3:47252028 | 14315 | 3:47485150 | 14333 | 3:47811507 | 14352 | 3:48155117 | 14353 | 3:48159568 |
| 14363 | 3:48456906 | 14367 | 3:48577665 | 14372 | 3:48681437 | 14374 | 3:48741921 | 14385 | 3:48923622 |
| 14390 | 3:48954466 | 14403 | 3:49283986 | 14413 | 3:49441533 | 14414 | 3:49442661 | 14417 | 3:49485613 |
| 14418 | 3:49491032 | 14419 | 3:49499289 | 14474 | 3:49732105 | 14477 | 3:49835535 | 14478 | 3:49879024 |
| 14479 | 3:49897610 | 14501 | 3:50633952 | 14516 | 3:51122225 | 14527 | 3:51610699 | 14530 | 3:51645901 |
| 14532 | 3:51683713 | 14534 | 3:51740729 | 14535 | 3:51755409 | 14603 | 3:53343252 | 14616 | 3:53703853 |
| 14626 | 3:54144822 | 14628 | 3:54149746 | 14629 | 3:54167611 | 14630 | 3:54178592 | 14631 | 3:54185471 |
| 14632 | 3:54186364 | 14633 | 3:54188660 | 14634 | 3:54201062 | 14638 | 3:54367531 | 14649 | 3:55014493 |
| 14650 | 3:55022327 | 14708 | 3:56670745 | 14714 | 3:56924360 | 14716 | 3:56934727 | 14717 | 3:56936672 |
| 14719 | 3:56953053 | 14721 | 3:56999586 | 14723 | 3:57032150 | 14727 | 3:57245572 | 14745 | 3:57799143 |
| 14749 | 3:57886444 | 14757 | 3:58161126 | 14767 | 3:58455621 | 14770 | 3:58489145 | 14772 | 3:58507559 |
| 14776 | 3:58545248 | 14787 | 3:58913251 | 14791 | 3:59017336 | 14795 | 3:59162716 | 14796 | 3:59207859 |
| 14816 | 3:59758400 | 14821 | 3:60026204 | 14826 | 3:60180066 | 14831 | 3:60261618 | 14849 | 3:60625549 |
| 14858 | 3:60737754 | 14867 | 3:60914592 | 14869 | 3:60962012 | 14888 | 3:61496668 | 14893 | 3:61744158 |
| 14896 | 3:61858795 | 14904 | 3:62095195 | 14910 | 3:62278399 | 14914 | 3:62359529 | 14933 | 3:62854439 |
| 14945 | 3:63290027 | 14972 | 3:63993039 | 14976 | 3:64069515 | 14991 | 3:64651171 | 14995 | 3:64790813 |
| 15006 | 3:65095548 | 15008 | 3:65157057 | 15028 | 3:65566921 | 15032 | 3:65663032 | 15057 | 3:66345116 |
| 15058 | 3:66351139 | 15061 | 3:66398657 | 15066 | 3:66502134 | 15070 | 3:66529229 | 15078 | 3:66637168 |
| 15083 | 3:66838529 | 15089 | 3:66948480 | 15091 | 3:66974396 | 15092 | 3:66999361 | 15093 | 3:67031006 |
| 15095 | 3:67168089 | 15102 | 3:67318712 | 15109 | 3:67473906 | 15116 | 3:67670465 | 15134 | 3:68047509 |
| 15140 | 3:68228132 | 15141 | 3:68228231 | 15142 | 3:68276086 | 15143 | 3:68302197 | 15145 | 3:68316834 |
| 15149 | 3:68422911 | 15151 | 3:68443638 | 15156 | 3:68551519 | 15158 | 3:68594137 | 15165 | 3:68727455 |
| 15166 | 3:68788461 | 15173 | 3:68973246 | 15174 | 3:69004319 | 15177 | 3:69066161 | 15184 | 3:69392637 |
| 15226 | 3:70427777 | 15229 | 3:70602082 | 15230 | 3:70652398 | 15255 | 3:71704029 | 15272 | 3:72307570 |
| 15273 | 3:72307597 | 15294 | 3:73250579 | 15305 | 3:73571608 | 15309 | 3:73698701 | 15310 | 3:73698721 |
| 15316 | 3:73904881 | 15317 | 3:73909382 | 15324 | 3:74202751 | 15327 | 3:74264677 | 15345 | 3:74829928 |
| 15349 | 3:74963727 | 15355 | 3:75071726 | 15364 | 3:75370499 | 15370 | 3:75506540 | 15374 | 3:75649317 |
| 15375 | 3:75649320 | 15376 | 3:75649336 | 15377 | 3:75649383 | 15398 | 3:76298830 | 15406 | 3:76468869 |
| 15415 | 3:76956223 | 15417 | 3:77030221 | 15423 | 3:77162998 | 15425 | 3:77178882 | 15434 | 3:77402844 |
| 15435 | 3:77409935 | 15438 | 3:77489908 | 15441 | 3:77571521 | 15449 | 3:77850286 | 15459 | 3:78097452 |
| 15465 | 3:78290774 | 15468 | 3:78420363 | 15473 | 3:78568977 | 15474 | 3:78569227 | 15475 | 3:78569835 |
| 15478 | 3:78642264 | 15479 | 3:78662366 | 15481 | 3:78785400 | 15489 | 3:79003182 | 15491 | 3:79007539 |
| 15497 | 3:79179242 | 15510 | 3:79464743 | 15523 | 3:79956010 | 15525 | 3:79973859 | 15531 | 3:80163495 |
| 15537 | 3:80491657 | 15540 | 3:80549623 | 15548 | 3:80733840 | 15556 | 3:80952119 | 15557 | 3:80954660 |
| 15558 | 3:80954761 | 15560 | 3:80999275 | 15566 | 3:81103725 | 15578 | 3:81314640 | 15580 | 3:81343738 |
| 15582 | 3:81436844 | 15584 | 3:81464927 | 15597 | 3:81749835 | 15613 | 3:82402375 | 15616 | 3:82454574 |
| 15620 | 3:82503792 | 15655 | 3:83636824 | 15658 | 3:83652637 | 15673 | 3:84036094 | 15680 | 3:84195762 |
| 15694 | 3:84534786 | 15695 | 3:84550534 | 15697 | 3:84560190 | 15726 | 3:84721616 | 15747 | 3:85077748 |
| 15751 | 3:85284944 | 15755 | 3:85429614 | 15756 | 3:85429668 | 15757 | 3:85429696 | 15775 | 3:85848368 |
| 15783 | 3:85978048 | 15791 | 3:86156348 | 15797 | 3:86290419 | 15800 | 3:86312689 | 15801 | 3:86312736 |
| 15805 | 3:86366796 | 15811 | 3:86584920 | 15815 | 3:86652496 | 15827 | 3:86872323 | 15840 | 3:87228581 |
| 15843 | 3:87295571 | 15847 | 3:87349602 | 15848 | 3:87379701 | 15929 | 3:88997759 | 15930 | 3:89044393 |
| 15932 | 3:89092242 | 15936 | 3:89255790 | 15957 | 3:89639867 | 15963 | 3:89717766 | 15964 | 3:89722428 |
| 15966 | 3:89773983 | 15968 | 3:89792849 | 15971 | 3:89858003 | 15972 | 3:89858025 | 15973 | 3:89861756 |
| 15974 | 3:89888858 | 15979 | 3:89947137 | 15983 | 3:90125909 | 15987 | 3:90218397 | 15996 | 3:90458410 |
| 15998 | 3:90490960 | 16012 | 3:90774079 | 16022 | 3:90916704 | 16028 | 3:91085209 | 16032 | 3:91177765 |
| 16038 | 3:91342742 | 16039 | 3:91349417 | 16047 | 3:91577320 | 16050 | 3:91662702 | 16056 | 3:91779733 |
| 16057 | 3:91821560 | 16058 | 3:91850874 | 16061 | 3:91944104 | 16064 | 3:92012709 | 16066 | 3:92056509 |
| 16072 | 3:92226650 | 16083 | 3:92532036 | 16092 | 3:92747667 | 16093 | 3:92757686 | 16096 | 3:92813097 |
| 16103 | 3:92955459 | 16105 | 3:92979991 | 16112 | 3:93070031 | 16115 | 3:93079352 | 16117 | 3:93087724 |
| 16123 | 3:93219307 | 16124 | 3:93219814 | 16125 | 3:93220543 | 16126 | 3:93226524 | 16127 | 3:93235948 |
| 16128 | 3:93314047 | 16130 | 3:93345899 | 16143 | 3:93782247 | 16144 | 3:93782331 | 16145 | 3:93782436 |
| 16159 | 3:94060442 | 16165 | 3:94226523 | 16178 | 3:94716721 | 16180 | 3:94726765 | 16183 | 3:94823429 |
| 16184 | 3:94866313 | 16186 | 3:94911090 | 16188 | 3:94976568 | 16190 | 3:95036766 | 16230 | 3:96202292 |
| 16238 | 3:96579309 | 16242 | 3:96636004 | 16247 | 3:96785820 | 16248 | 3:96830575 | 16254 | 3:96975581 |
| 16260 | 3:97123954 | 16269 | 3:97365473 | 16274 | 3:97457441 | 16275 | 3:97473661 | 16278 | 3:97526789 |
| 16290 | 3:97708909 | 16301 | 3:97930159 | 16305 | 3:98116083 | 16309 | 3:98295744 | 16313 | 3:98349294 |
| 16317 | 3:98403571 | 16321 | 3:98561508 | 16335 | 3:98923450 | 16337 | 3:98978448 | 16338 | 3:98978458 |
| 16348 | 3:99342353 | 16350 | 3:99349479 | 16351 | 3:99356345 | 16352 | 3:99360354 | 16353 | 3:99377234 |
| 16358 | 3:99519922 | 16368 | 3:99751257 | 16369 | 3:99765693 | 16370 | 3:99771380 | 16371 | 3:99805386 |
| 16372 | 3:99811235 | 16373 | 3:99811520 | 16374 | 3:99818061 | 16375 | 3:99831451 | 16376 | 3:99840460 |
| 16377 | 3:99840480 | 16378 | 3:99884150 | 16381 | 3:99928886 | 16383 | 3:99953595 | 16387 | 3:100006117 |
| 16388 | 3:100006731 | 16391 | 3:100135156 | 16393 | 3:100148664 | 16403 | 3:100386736 | 16405 | 3:100413711 |
| 16406 | 3:100417680 | 16407 | 3:100429883 | 16410 | 3:100503760 | 16411 | 3:100519527 | 16412 | 3:100527924 |
| 16413 | 3:100532091 | 16415 | 3:100578191 | 16417 | 3:100604376 | 16418 | 3:100616906 | 16420 | 3:100649238 |
| 16421 | 3:100655322 | 16422 | 3:100660031 | 16425 | 3:100706971 | 16426 | 3:100710303 | 16435 | 3:100839974 |
| 16440 | 3:101045558 | 16449 | 3:101184420 | 16456 | 3:101301924 | 16457 | 3:101328497 | 16462 | 3:101381082 |
| 16496 | 3:102252547 | 16500 | 3:102323975 | 16502 | 3:102346918 | 16504 | 3:102360619 | 16505 | 3:102430768 |
| 16506 | 3:102432444 | 16515 | 3:102664478 | 16516 | 3:102684885 | 16517 | 3:102684933 | 16518 | 3:102684985 |
| 16520 | 3:102736091 | 16521 | 3:102753266 | 16524 | 3:102804450 | 16529 | 3:102845961 | 16530 | 3:102846036 |
| 16531 | 3:102853282 | 16536 | 3:102881866 | 16555 | 3:103364021 | 16560 | 3:103438363 | 16564 | 3:103569295 |
| 16566 | 3:103617298 | 16567 | 3:103655132 | 16569 | 3:103692819 | 16571 | 3:103732748 | 16573 | 3:103756143 |
| 16578 | 3:103836979 | 16581 | 3:103923977 | 16583 | 3:104007365 | 16584 | 3:104022881 | 16590 | 3:104089157 |
| 16598 | 3:104333611 | 16610 | 3:104577770 | 16612 | 3:104588926 | 16614 | 3:104626150 | 16616 | 3:104664666 |
| 16618 | 3:104700803 | 16621 | 3:104722133 | 16622 | 3:104733085 | 16624 | 3:104818821 | 16629 | 3:104879046 |
| 16641 | 3:105195217 | 16642 | 3:105195508 | 16644 | 3:105197594 | 16645 | 3:105207363 | 16648 | 3:105223366 |
| 16649 | 3:105224129 | 16650 | 3:105224197 | 16656 | 3:105344373 | 16657 | 3:105346169 | 16658 | 3:105386965 |
| 16659 | 3:105397156 | 16660 | 3:105399284 | 16661 | 3:105401478 | 16664 | 3:105416679 | 16665 | 3:105419956 |
| 16666 | 3:105420955 | 16668 | 3:105522467 | 16669 | 3:105528154 | 16670 | 3:105530865 | 16671 | 3:105550247 |
| 16672 | 3:105553161 | 16673 | 3:105557267 | 16675 | 3:105587522 | 16676 | 3:105587615 | 16677 | 3:105588435 |
| 16678 | 3:105589110 | 16679 | 3:105589417 | 16680 | 3:105589499 | 16681 | 3:105589515 | 16682 | 3:105589567 |
| 16683 | 3:105589840 | 16686 | 3:105615307 | 16687 | 3:105623790 | 16688 | 3:105633378 | 16689 | 3:105633804 |
| 16691 | 3:105644182 | 16692 | 3:105654662 | 16693 | 3:105674940 | 16696 | 3:105745106 | 16701 | 3:105803674 |
| 16702 | 3:105805027 | 16705 | 3:105897358 | 16706 | 3:105897511 | 16707 | 3:105897845 | 16708 | 3:105897970 |
| 16709 | 3:105908966 | 16710 | 3:105909058 | 16712 | 3:105935499 | 16713 | 3:105956333 | 16717 | 3:106020186 |
| 16724 | 3:106135737 | 16731 | 3:106244085 | 16735 | 3:106337682 | 16745 | 3:106561725 | 16746 | 3:106561875 |
| 16747 | 3:106585197 | 16748 | 3:106591729 | 16754 | 3:106655656 | 16755 | 3:106668839 | 16765 | 3:106845702 |
| 16771 | 3:107103522 | 16783 | 3:107354102 | 16784 | 3:107375067 | 16785 | 3:107375088 | 16786 | 3:107375142 |
| 16789 | 3:107445270 | 16791 | 3:107477138 | 16800 | 3:107604142 | 16807 | 3:107789881 | 16817 | 3:107937893 |
| 16819 | 3:107940715 | 16830 | 3:108203613 | 16831 | 3:108224103 | 16833 | 3:108305450 | 16835 | 3:108345650 |
| 16850 | 3:108755292 | 16851 | 3:108756407 | 16852 | 3:108760658 | 16853 | 3:108761540 | 16854 | 3:108763978 |
| 16855 | 3:108764835 | 16856 | 3:108773793 | 16857 | 3:108777623 | 16858 | 3:108778270 | 16860 | 3:108788102 |
| 16861 | 3:108817854 | 16862 | 3:108824614 | 16864 | 3:108842377 | 16885 | 3:109426329 | 16888 | 3:109430792 |
| 16889 | 3:109430841 | 16899 | 3:109606532 | 16900 | 3:109656896 | 16916 | 3:109969045 | 16920 | 3:110020722 |
| 16921 | 3:110026915 | 16923 | 3:110036182 | 16927 | 3:110153404 | 16946 | 3:110780122 | 16947 | 3:110781851 |
| 16948 | 3:110783875 | 16949 | 3:110784912 | 16950 | 3:110786097 | 16954 | 3:110849640 | 16978 | 3:111418313 |
| 16986 | 3:111526183 | 16989 | 3:111580049 | 17003 | 3:111889624 | 17019 | 3:112192839 | 17025 | 3:112323001 |
| 17026 | 3:112345897 | 17032 | 3:112397006 | 17046 | 3:112761486 | 17047 | 3:112761940 | 17050 | 3:112794864 |
| 17058 | 3:113036203 | 17062 | 3:113127829 | 17063 | 3:113131422 | 17064 | 3:113146141 | 17072 | 3:113331205 |
| 17073 | 3:113331220 | 17074 | 3:113337934 | 17075 | 3:113341660 | 17078 | 3:113407286 | 17081 | 3:113453006 |
| 17083 | 3:113518551 | 17084 | 3:113518636 | 17085 | 3:113524422 | 17089 | 3:113563964 | 17090 | 3:113563979 |
| 17091 | 3:113563997 | 17092 | 3:113564006 | 17093 | 3:113564076 | 17094 | 3:113564096 | 17095 | 3:113564107 |
| 17096 | 3:113585771 | 17097 | 3:113585795 | 17098 | 3:113585799 | 17100 | 3:113586101 | 17101 | 3:113586251 |
| 17102 | 3:113590170 | 17120 | 3:114115462 | 17126 | 3:114165101 | 17137 | 3:114384157 | 17141 | 3:114446007 |
| 17144 | 3:114496084 | 17146 | 3:114508185 | 17149 | 3:114543461 | 17150 | 3:114548928 | 17152 | 3:114594768 |
| 17156 | 3:114618765 | 17160 | 3:114704691 | 17164 | 3:114748408 | 17175 | 3:114802567 | 17185 | 3:114881902 |
| 17190 | 3:115000401 | 17193 | 3:115052234 | 17199 | 3:115107550 | 17200 | 3:115120882 | 17208 | 3:115363288 |
| 17210 | 3:115374146 | 17213 | 3:115397893 | 17222 | 3:115484538 | 17225 | 3:115543657 | 17227 | 3:115544750 |
| 17231 | 3:115602232 | 17232 | 3:115602518 | 17237 | 3:115733885 | 17238 | 3:115814948 | 17243 | 3:115936238 |
| 17246 | 3:115967688 | 17262 | 3:116221838 | 17264 | 3:116272992 | 17270 | 3:116347067 | 17282 | 3:116562510 |
| 17291 | 3:116772922 | 17299 | 3:116861866 | 17310 | 3:116928313 | 17314 | 3:116981402 | 17315 | 3:116981583 |
| 17316 | 3:116981638 | 17320 | 3:117000919 | 17323 | 3:117031812 | 17324 | 3:117049753 | 17326 | 3:117070587 |
| 17330 | 3:117123078 | 17331 | 3:117126904 | 17334 | 3:117149901 | 17337 | 3:117209899 | 17338 | 3:117209960 |
| 17341 | 3:117278310 | 17342 | 3:117283114 | 17344 | 3:117317452 | 17345 | 3:117325008 | 17353 | 3:117578080 |
| 17360 | 3:117649528 | 17361 | 3:117649537 | 17362 | 3:117662078 | 17365 | 3:117673624 | 17366 | 3:117696207 |
| 17370 | 3:117709813 | 17394 | 3:118128778 | 17401 | 3:118223118 | 17403 | 3:118288926 | 17404 | 3:118289376 |
| 17405 | 3:118289408 | 17406 | 3:118289679 | 17411 | 3:118387538 | 17420 | 3:118548276 | 17426 | 3:118615619 |
| 17436 | 3:118755947 | 17440 | 3:118806132 | 17449 | 3:118883045 | 17457 | 3:118964562 | 17461 | 3:119094347 |
| 17467 | 3:119427638 | 17472 | 3:119648530 | 17475 | 3:119673122 | 17476 | 3:119709097 | 17481 | 3:119787932 |
| 17486 | 3:119868121 | 17499 | 3:120268455 | 17502 | 3:120306744 | 17503 | 3:120306964 | 17510 | 3:120576521 |
| 17515 | 3:120695918 | 17522 | 3:120790650 | 17553 | 4:1446328 | 17648 | 4:4173794 | 17649 | 4:4184943 |
| 17663 | 4:4533982 | 17666 | 4:4582744 | 17674 | 4:4681609 | 17675 | 4:4681672 | 17679 | 4:4733530 |
| 17686 | 4:4818261 | 17691 | 4:4871624 | 17692 | 4:4931247 | 17693 | 4:4943634 | 17701 | 4:5077162 |
| 17703 | 4:5134591 | 17708 | 4:5202035 | 17714 | 4:5319445 | 17715 | 4:5319859 | 17721 | 4:5377091 |
| 17722 | 4:5395548 | 17724 | 4:5422821 | 17725 | 4:5437743 | 17726 | 4:5441832 | 17756 | 4:6207303 |
| 17787 | 4:7009102 | 17788 | 4:7009111 | 17792 | 4:7073207 | 17793 | 4:7106157 | 17799 | 4:7247371 |
| 17808 | 4:7354530 | 17814 | 4:7445669 | 17820 | 4:7632416 | 17821 | 4:7658763 | 17822 | 4:7658787 |
| 17823 | 4:7658831 | 17824 | 4:7658948 | 17825 | 4:7658962 | 17827 | 4:7697381 | 17835 | 4:7933911 |
| 17836 | 4:7945782 | 17837 | 4:7996659 | 17840 | 4:8046313 | 17844 | 4:8179768 | 17847 | 4:8246510 |
| 17850 | 4:8272646 | 17853 | 4:8420098 | 17854 | 4:8435396 | 17856 | 4:8445348 | 17859 | 4:8506844 |
| 17883 | 4:9151646 | 17891 | 4:9271969 | 17893 | 4:9323578 | 17896 | 4:9385962 | 17898 | 4:9407447 |
| 17900 | 4:9444470 | 17901 | 4:9474683 | 17902 | 4:9477621 | 17904 | 4:9485074 | 17905 | 4:9486301 |
| 17906 | 4:9488068 | 17907 | 4:9490132 | 17908 | 4:9490936 | 17909 | 4:9492909 | 17920 | 4:9817295 |
| 17949 | 4:10163330 | 17953 | 4:10231259 | 17963 | 4:10387935 | 17973 | 4:10679109 | 17996 | 4:11190947 |
| 17997 | 4:11191071 | 18000 | 4:11265836 | 18001 | 4:11269876 | 18002 | 4:11271703 | 18007 | 4:11332982 |
| 18008 | 4:11339823 | 18009 | 4:11344256 | 18010 | 4:11344907 | 18029 | 4:11936615 | 18050 | 4:12576634 |
| 18052 | 4:12673015 | 18061 | 4:12802609 | 18063 | 4:12826295 | 18066 | 4:12860940 | 18072 | 4:13010035 |
| 18091 | 4:13579464 | 18092 | 4:13579504 | 18093 | 4:13579532 | 18098 | 4:13707039 | 18102 | 4:13801041 |
| 18106 | 4:14002326 | 18110 | 4:14035164 | 18111 | 4:14061507 | 18112 | 4:14079747 | 18148 | 4:15337479 |
| 18153 | 4:15469076 | 18154 | 4:15470050 | 18160 | 4:15586640 | 18182 | 4:16245146 | 18200 | 4:16761268 |
| 18210 | 4:17040967 | 18221 | 4:17237895 | 18235 | 4:17572154 | 18236 | 4:17572159 | 18237 | 4:17572202 |
| 18238 | 4:17572213 | 18256 | 4:18084841 | 18272 | 4:18585854 | 18277 | 4:18655237 | 18279 | 4:18662663 |
| 18280 | 4:18669066 | 18282 | 4:18685097 | 18284 | 4:18697683 | 18286 | 4:18705074 | 18287 | 4:18707492 |
| 18293 | 4:18889879 | 18325 | 4:19569259 | 18363 | 4:19999023 | 18368 | 4:20111420 | 18373 | 4:20255515 |
| 18379 | 4:20329251 | 18380 | 4:20329405 | 18397 | 4:20796684 | 18398 | 4:20802780 | 18399 | 4:20804901 |
| 18419 | 4:21160009 | 18420 | 4:21160045 | 18422 | 4:21189572 | 18424 | 4:21207059 | 18425 | 4:21213102 |
| 18460 | 4:22190378 | 18463 | 4:22352000 | 18469 | 4:22526571 | 18488 | 4:22913622 | 18498 | 4:23160284 |
| 18510 | 4:23595303 | 18521 | 4:23872526 | 18523 | 4:23891166 | 18526 | 4:23927655 | 18527 | 4:23946714 |
| 18536 | 4:24333008 | 18549 | 4:24650631 | 18554 | 4:24810069 | 18557 | 4:24885290 | 18565 | 4:25097829 |
| 18566 | 4:25109810 | 18567 | 4:25127084 | 18583 | 4:25647682 | 18587 | 4:25742989 | 18590 | 4:25907759 |
| 18591 | 4:25907762 | 18594 | 4:25924624 | 18609 | 4:26257023 | 18621 | 4:26648480 | 18627 | 4:26786277 |
| 18641 | 4:27097020 | 18642 | 4:27127515 | 18654 | 4:27476944 | 18662 | 4:27629208 | 18666 | 4:27698262 |
| 18681 | 4:28186796 | 18682 | 4:28217559 | 18686 | 4:28338304 | 18690 | 4:28410444 | 18691 | 4:28434228 |
| 18703 | 4:28719533 | 18704 | 4:28738300 | 18712 | 4:28975908 | 18714 | 4:29080932 | 18716 | 4:29142901 |
| 18745 | 4:30297344 | 18752 | 4:30430428 | 18753 | 4:30430450 | 18754 | 4:30430542 | 18755 | 4:30432018 |
| 18757 | 4:30501662 | 18784 | 4:31141033 | 18790 | 4:31306787 | 18795 | 4:31471170 | 18797 | 4:31535187 |
| 18801 | 4:31680539 | 18809 | 4:31817533 | 18810 | 4:31854356 | 18814 | 4:31949743 | 18854 | 4:33211684 |
| 18858 | 4:33325825 | 18869 | 4:33588518 | 18873 | 4:33639372 | 18874 | 4:33652107 | 18876 | 4:33739841 |
| 18878 | 4:33761684 | 18880 | 4:33789343 | 18887 | 4:33926324 | 18894 | 4:34109934 | 18913 | 4:34771597 |
| 18921 | 4:35053751 | 18923 | 4:35162460 | 18934 | 4:35468668 | 18964 | 4:36420563 | 18970 | 4:36597277 |
| 18971 | 4:36657445 | 18981 | 4:36944816 | 18996 | 4:37260138 | 19008 | 4:37691142 | 19021 | 4:38080777 |
| 19023 | 4:38088154 | 19027 | 4:38196269 | 19029 | 4:38253124 | 19034 | 4:38381571 | 19048 | 4:38785910 |
| 19058 | 4:39096980 | 19077 | 4:39839146 | 19079 | 4:39869029 | 19096 | 4:40085698 | 19097 | 4:40100668 |
| 19111 | 4:40502117 | 19112 | 4:40545187 | 19120 | 4:40660943 | 19122 | 4:40678229 | 19128 | 4:40850556 |
| 19131 | 4:40925102 | 19136 | 4:40978581 | 19137 | 4:40990306 | 19138 | 4:40990370 | 19139 | 4:40990413 |
| 19140 | 4:40994417 | 19143 | 4:41012545 | 19144 | 4:41012589 | 19145 | 4:41012593 | 19155 | 4:41242597 |
| 19171 | 4:42001110 | 19176 | 4:42079882 | 19179 | 4:42269505 | 19186 | 4:42478244 | 19198 | 4:42862322 |
| 19217 | 4:43233221 | 19218 | 4:43233239 | 19219 | 4:43233296 | 19220 | 4:43233323 | 19221 | 4:43233412 |
| 19223 | 4:43330695 | 19234 | 4:43684052 | 19242 | 4:43934934 | 19244 | 4:43942237 | 19248 | 4:44063960 |
| 19253 | 4:44178421 | 19256 | 4:44256983 | 19257 | 4:44268015 | 19260 | 4:44305806 | 19268 | 4:44547450 |
| 19282 | 4:45052002 | 19298 | 4:45602329 | 19311 | 4:46002303 | 19315 | 4:46099926 | 19317 | 4:46129459 |
| 19329 | 4:46600207 | 19340 | 4:46830510 | 19345 | 4:46921985 | 19346 | 4:46925441 | 19347 | 4:46925682 |
| 19348 | 4:46943050 | 19349 | 4:46943107 | 19350 | 4:46943131 | 19363 | 4:47258425 | 19379 | 4:47479517 |
| 19383 | 4:47511989 | 19386 | 4:47569946 | 19397 | 4:47904922 | 19399 | 4:48028783 | 19403 | 4:48241131 |
| 19420 | 4:48780844 | 19426 | 4:49017869 | 19428 | 4:49022872 | 19430 | 4:49035315 | 19431 | 4:49035320 |
| 19432 | 4:49035381 | 19433 | 4:49035395 | 19434 | 4:49035467 | 19435 | 4:49037953 | 19437 | 4:49045621 |
| 19438 | 4:49045631 | 19439 | 4:49045737 | 19440 | 4:49045753 | 19441 | 4:49045756 | 19442 | 4:49045852 |
| 19444 | 4:49061063 | 19445 | 4:49062116 | 19447 | 4:49107209 | 19452 | 4:49181169 | 19456 | 4:49269697 |
| 19457 | 4:49284053 | 19467 | 4:49523230 | 19478 | 4:49842196 | 19482 | 4:49964915 | 19486 | 4:50057099 |
| 19489 | 4:50248085 | 19490 | 4:50345064 | 19496 | 4:50538062 | 19515 | 4:51250626 | 19517 | 4:51303879 |
| 19526 | 4:51543766 | 19532 | 4:51713354 | 19581 | 4:53246820 | 19594 | 4:53516423 | 19599 | 4:53675011 |
| 19614 | 4:54062446 | 19622 | 4:54498204 | 19634 | 4:54973210 | 19638 | 4:55064494 | 19643 | 4:55358812 |
| 19646 | 4:55397907 | 19658 | 4:55841154 | 19659 | 4:55864590 | 19660 | 4:55873977 | 19663 | 4:55914722 |
| 19665 | 4:55965169 | 19671 | 4:56042508 | 19674 | 4:56091771 | 19683 | 4:56311977 | 19689 | 4:56597023 |
| 19691 | 4:56614952 | 19696 | 4:56795183 | 19706 | 4:57053471 | 19711 | 4:57121533 | 19713 | 4:57264096 |
| 19722 | 4:57443352 | 19726 | 4:57549751 | 19727 | 4:57552140 | 19730 | 4:57682773 | 19735 | 4:57810438 |
| 19764 | 4:58952251 | 19771 | 4:59151344 | 19778 | 4:59234606 | 19796 | 4:59761295 | 19798 | 4:59797184 |
| 19799 | 4:59797205 | 19817 | 4:60407425 | 19823 | 4:60610913 | 19829 | 4:60822961 | 19835 | 4:61004115 |
| 19843 | 4:61150481 | 19844 | 4:61176028 | 19845 | 4:61188646 | 19868 | 4:61850527 | 19876 | 4:62043721 |
| 19886 | 4:62197348 | 19901 | 4:62758226 | 19902 | 4:62762359 | 19903 | 4:62769531 | 19910 | 4:62994404 |
| 19911 | 4:63001098 | 19919 | 4:63185750 | 19922 | 4:63289063 | 19926 | 4:63398159 | 19932 | 4:63595012 |
| 19945 | 4:63872067 | 19946 | 4:63887485 | 19948 | 4:63898910 | 19949 | 4:63913879 | 19957 | 4:64206739 |
| 19966 | 4:64550273 | 19968 | 4:64596464 | 19969 | 4:64598653 | 19970 | 4:64633068 | 19997 | 4:65579692 |
| 19998 | 4:65585572 | 20000 | 4:65586522 | 20004 | 4:65662118 | 20005 | 4:65663680 | 20006 | 4:65663924 |
| 20011 | 4:65757109 | 20016 | 4:65871790 | 20018 | 4:65887212 | 20020 | 4:65935466 | 20026 | 4:66031691 |
| 20030 | 4:66126342 | 20032 | 4:66167545 | 20040 | 4:66351372 | 20044 | 4:66424351 | 20058 | 4:66948425 |
| 20064 | 4:67093419 | 20079 | 4:67478622 | 20089 | 4:67825161 | 20093 | 4:67886242 | 20098 | 4:67941747 |
| 20103 | 4:68103259 | 20106 | 4:68130974 | 20112 | 4:68281786 | 20118 | 4:68525805 | 20138 | 4:69316258 |
| 20146 | 4:69461895 | 20150 | 4:69590761 | 20153 | 4:69624135 | 20162 | 4:69834879 | 20168 | 4:69953830 |
| 20177 | 4:70190883 | 20179 | 4:70206948 | 20181 | 4:70239423 | 20184 | 4:70315106 | 20197 | 4:70629516 |
| 20200 | 4:70650674 | 20207 | 4:70942839 | 20208 | 4:70955859 | 20210 | 4:71014142 | 20212 | 4:71044933 |
| 20241 | 4:71963805 | 20242 | 4:71995369 | 20247 | 4:72082867 | 20250 | 4:72155086 | 20265 | 4:72831364 |
| 20266 | 4:72831400 | 20278 | 4:73141876 | 20280 | 4:73230353 | 20281 | 4:73230433 | 20282 | 4:73230506 |
| 20287 | 4:73356120 | 20295 | 4:73677765 | 20320 | 4:74355629 | 20326 | 4:74574092 | 20327 | 4:74575187 |
| 20328 | 4:74575293 | 20332 | 4:74690642 | 20336 | 4:74750397 | 20337 | 4:74775088 | 20339 | 4:74788299 |
| 20347 | 4:74921800 | 20348 | 4:74931461 | 20349 | 4:74937132 | 20351 | 4:74962440 | 20355 | 4:75035303 |
| 20360 | 4:75176025 | 20375 | 4:75585274 | 20376 | 4:75596693 | 20395 | 4:75964360 | 20410 | 4:76300291 |
| 20411 | 4:76303426 | 20427 | 4:76654098 | 20428 | 4:76665449 | 20431 | 4:76826893 | 20434 | 4:77005397 |
| 20438 | 4:77038646 | 20439 | 4:77114794 | 20440 | 4:77131669 | 20441 | 4:77141818 | 20443 | 4:77158468 |
| 20446 | 4:77222263 | 20447 | 4:77232107 | 20448 | 4:77235523 | 20449 | 4:77272270 | 20453 | 4:77462163 |
| 20459 | 4:77550845 | 20460 | 4:77550847 | 20461 | 4:77550961 | 20463 | 4:77552548 | 20464 | 4:77552605 |
| 20465 | 4:77552647 | 20466 | 4:77562640 | 20467 | 4:77564937 | 20468 | 4:77565007 | 20469 | 4:77565031 |
| 20470 | 4:77612846 | 20474 | 4:77703279 | 20484 | 4:78046476 | 20493 | 4:78275533 | 20500 | 4:78501007 |
| 20516 | 4:79081070 | 20532 | 4:79524078 | 20534 | 4:79581997 | 20535 | 4:79582037 | 20570 | 4:80210945 |
| 20571 | 4:80211747 | 20575 | 4:80312765 | 20577 | 4:80342472 | 20578 | 4:80347612 | 20603 | 4:81058244 |
| 20606 | 4:81115882 | 20608 | 4:81125829 | 20612 | 4:81203512 | 20613 | 4:81222120 | 20615 | 4:81291786 |
| 20624 | 4:81541207 | 20646 | 4:82094311 | 20656 | 4:82418721 | 20664 | 4:82649849 | 20665 | 4:82649930 |
| 20668 | 4:82720591 | 20669 | 4:82720596 | 20670 | 4:82720610 | 20671 | 4:82720657 | 20672 | 4:82720686 |
| 20673 | 4:82720715 | 20679 | 4:82824766 | 20696 | 4:83387242 | 20697 | 4:83420381 | 20699 | 4:83506707 |
| 20700 | 4:83540447 | 20701 | 4:83605440 | 20725 | 4:84374337 | 20748 | 4:84998314 | 20767 | 4:85529127 |
| 20769 | 4:85585852 | 20770 | 4:85589724 | 20771 | 4:85609908 | 20783 | 4:85887734 | 20784 | 4:85902484 |
| 20791 | 4:86035429 | 20794 | 4:86107048 | 20805 | 4:86429759 | 20807 | 4:86434813 | 20808 | 4:86453759 |
| 20812 | 4:86588536 | 20827 | 4:86869600 | 20831 | 4:87015587 | 20832 | 4:87025872 | 20833 | 4:87026389 |
| 20847 | 4:87544345 | 20861 | 4:87862791 | 20869 | 4:87966655 | 20870 | 4:87988315 | 20871 | 4:88003339 |
| 20874 | 4:88190387 | 20876 | 4:88214757 | 20877 | 4:88230965 | 20878 | 4:88267363 | 20883 | 4:88482339 |
| 20916 | 4:89540510 | 20919 | 4:89600401 | 20920 | 4:89600435 | 20921 | 4:89600533 | 20925 | 4:89717462 |
| 20926 | 4:89720129 | 20997 | 4:91292909 | 21000 | 4:91358381 | 21011 | 4:91589372 | 21017 | 4:91792117 |
| 21021 | 4:91837317 | 21032 | 4:92071091 | 21034 | 4:92150036 | 21036 | 4:92281567 | 21038 | 4:92292551 |
| 21039 | 4:92298261 | 21040 | 4:92299550 | 21041 | 4:92307140 | 21042 | 4:92315009 | 21044 | 4:92351942 |
| 21049 | 4:92444591 | 21058 | 4:92540924 | 21062 | 4:92634658 | 21065 | 4:92695366 | 21072 | 4:92893919 |
| 21083 | 4:93143125 | 21086 | 4:93270718 | 21088 | 4:93325674 | 21091 | 4:93338554 | 21092 | 4:93343119 |
| 21093 | 4:93349924 | 21094 | 4:93350879 | 21095 | 4:93352485 | 21096 | 4:93361239 | 21097 | 4:93361477 |
| 21098 | 4:93362689 | 21099 | 4:93368708 | 21100 | 4:93373657 | 21101 | 4:93376458 | 21102 | 4:93388339 |
| 21104 | 4:93445593 | 21107 | 4:93470782 | 21114 | 4:93581039 | 21121 | 4:93756393 | 21125 | 4:93956921 |
| 21138 | 4:94448413 | 21147 | 4:94698872 | 21148 | 4:94699823 | 21151 | 4:94815446 | 21152 | 4:94833611 |
| 21156 | 4:94917698 | 21157 | 4:94918493 | 21169 | 4:95254763 | 21170 | 4:95254765 | 21171 | 4:95254773 |
| 21172 | 4:95254787 | 21175 | 4:95294696 | 21178 | 4:95382163 | 21196 | 4:95826773 | 21198 | 4:95871966 |
| 21203 | 4:95901052 | 21207 | 4:95975966 | 21221 | 4:96011612 | 21233 | 4:96120257 | 21234 | 4:96121708 |
| 21235 | 4:96145894 | 21236 | 4:96192829 | 21237 | 4:96196621 | 21241 | 4:96307908 | 21249 | 4:96528346 |
| 21250 | 4:96528347 | 21285 | 4:96829783 | 21359 | 4:96940681 | 21365 | 4:96997475 | 21371 | 4:97113666 |
| 21416 | 4:97172490 | 21421 | 4:97266645 | 21444 | 4:97789995 | 21450 | 4:98087559 | 21454 | 4:98210913 |
| 21455 | 4:98212198 | 21457 | 4:98226178 | 21458 | 4:98226699 | 21459 | 4:98239198 | 21463 | 4:98381525 |
| 21464 | 4:98406747 | 21470 | 4:98545217 | 21472 | 4:98604946 | 21473 | 4:98617473 | 21474 | 4:98640953 |
| 21477 | 4:98765309 | 21482 | 4:98852463 | 21483 | 4:98862279 | 21492 | 4:99462535 | 21498 | 4:99648977 |
| 21499 | 4:99650381 | 21500 | 4:99652215 | 21509 | 4:99864130 | 21511 | 4:99877799 | 21513 | 4:99884272 |
| 21515 | 4:99907629 | 21518 | 4:100010231 | 21521 | 4:100048120 | 21522 | 4:100048151 | 21533 | 4:100235968 |
| 21558 | 4:100865583 | 21560 | 4:100891716 | 21564 | 4:100958472 | 21569 | 4:101138513 | 21580 | 4:101273627 |
| 21581 | 4:101278348 | 21582 | 4:101283299 | 21583 | 4:101286906 | 21584 | 4:101304065 | 21594 | 4:101562975 |
| 21595 | 4:101570088 | 21597 | 4:101615078 | 21599 | 4:101627868 | 21604 | 4:101697277 | 21606 | 4:101718193 |
| 21607 | 4:101720088 | 21608 | 4:101796334 | 21609 | 4:101817565 | 21612 | 4:102044239 | 21613 | 4:102053938 |
| 21623 | 4:102294876 | 21630 | 4:102465082 | 21631 | 4:102515585 | 21632 | 4:102515711 | 21633 | 4:102515830 |
| 21635 | 4:102567199 | 21636 | 4:102567675 | 21637 | 4:102571979 | 21638 | 4:102577247 | 21641 | 4:102679178 |
| 21650 | 4:102994861 | 21653 | 4:103066617 | 21656 | 4:103118208 | 21672 | 4:103464890 | 21673 | 4:103466759 |
| 21678 | 4:103589488 | 21691 | 4:103984758 | 21705 | 4:104406614 | 21713 | 4:104554874 | 21714 | 4:104639191 |
| 21719 | 4:104710790 | 21721 | 4:104731929 | 21726 | 4:104879688 | 21730 | 4:104955413 | 21739 | 4:105212815 |
| 21742 | 4:105331039 | 21743 | 4:105332159 | 21747 | 4:105369148 | 21753 | 4:105480650 | 21754 | 4:105480728 |
| 21755 | 4:105482792 | 21766 | 4:105750600 | 21767 | 4:105757195 | 21768 | 4:105785525 | 21773 | 4:106025379 |
| 21775 | 4:106025551 | 21780 | 4:106081660 | 21781 | 4:106085160 | 21782 | 4:106088179 | 21783 | 4:106088193 |
| 21784 | 4:106088224 | 21785 | 4:106088251 | 21786 | 4:106088286 | 21792 | 4:106177289 | 21796 | 4:106243037 |
| 21797 | 4:106246162 | 21798 | 4:106257365 | 21807 | 4:106459877 | 21808 | 4:106461237 | 21809 | 4:106461523 |
| 21810 | 4:106462584 | 21811 | 4:106463211 | 21812 | 4:106526851 | 21817 | 4:106674077 | 21820 | 4:106723818 |
| 21821 | 4:106724580 | 21822 | 4:106738338 | 21823 | 4:106768265 | 21827 | 4:106823063 | 21833 | 4:106915810 |
| 21835 | 4:106985422 | 21837 | 4:106991677 | 21838 | 4:106991678 | 21839 | 4:106991818 | 21840 | 4:106991819 |
| 21841 | 4:106993054 | 21842 | 4:106993083 | 21843 | 4:106993086 | 21844 | 4:106993163 | 21845 | 4:106993484 |
| 21846 | 4:106993491 | 21847 | 4:106993778 | 21848 | 4:106993951 | 21849 | 4:106993975 | 21850 | 4:106994109 |
| 21851 | 4:106994258 | 21852 | 4:106997706 | 21853 | 4:106998207 | 21857 | 4:107044582 | 21858 | 4:107070723 |
| 21859 | 4:107075139 | 21867 | 4:107202696 | 21868 | 4:107202723 | 21869 | 4:107202867 | 21870 | 4:107202876 |
| 21873 | 4:107293705 | 21874 | 4:107293767 | 21875 | 4:107293937 | 21876 | 4:107293975 | 21877 | 4:107293984 |
| 21878 | 4:107294409 | 21879 | 4:107294413 | 21884 | 4:107599054 | 21891 | 4:107760331 | 21896 | 4:107893188 |
| 21918 | 4:108666311 | 21919 | 4:108666329 | 21936 | 4:109206944 | 21996 | 4:110555410 | 21998 | 4:110627140 |
| 22003 | 4:110751708 | 22004 | 4:110751716 | 22005 | 4:110759035 | 22006 | 4:110759043 | 22007 | 4:110759085 |
| 22008 | 4:110759138 | 22009 | 4:110759193 | 22012 | 4:110870537 | 22017 | 4:110963804 | 22018 | 4:110997685 |
| 22021 | 4:111094939 | 22027 | 4:111132995 | 22030 | 4:111174187 | 22036 | 4:111290634 | 22041 | 4:111393405 |
| 22042 | 4:111393524 | 22060 | 4:111696373 | 22072 | 4:111836637 | 22075 | 4:111906579 | 22076 | 4:111910434 |
| 22077 | 4:111911436 | 22079 | 4:111972681 | 22085 | 4:112108098 | 22087 | 4:112136295 | 22090 | 4:112153870 |
| 22091 | 4:112155488 | 22093 | 4:112172784 | 22097 | 4:112238690 | 22100 | 4:112269261 | 22110 | 4:112424206 |
| 22112 | 4:112448562 | 22113 | 4:112452844 | 22117 | 4:112516104 | 22122 | 4:112599976 | 22124 | 4:112652763 |
| 22125 | 4:112660638 | 22126 | 4:112663309 | 22127 | 4:112664440 | 22128 | 4:112665144 | 22129 | 4:112671210 |
| 22130 | 4:112675707 | 22131 | 4:112676199 | 22132 | 4:112677340 | 22133 | 4:112681999 | 22136 | 4:112716249 |
| 22138 | 4:112739929 | 22139 | 4:112740655 | 22140 | 4:112741810 | 22141 | 4:112748029 | 22142 | 4:112749294 |
| 22143 | 4:112754237 | 22144 | 4:112755579 | 22145 | 4:112769671 | 22146 | 4:112770319 | 22147 | 4:112785850 |
| 22153 | 4:112842045 | 22154 | 4:112844573 | 22155 | 4:112855696 | 22157 | 4:112873667 | 22158 | 4:112873671 |
| 22159 | 4:112873791 | 22160 | 4:112874161 | 22162 | 4:112920884 | 22163 | 4:112920908 | 22164 | 4:112958565 |
| 22178 | 4:113478902 | 22184 | 4:113579221 | 22185 | 4:113579506 | 22186 | 4:113584318 | 22187 | 4:113584327 |
| 22188 | 4:113584399 | 22189 | 4:113584405 | 22190 | 4:113587147 | 22199 | 4:113617600 | 22200 | 4:113617762 |
| 22201 | 4:113622976 | 22220 | 4:113685923 | 22221 | 4:113693137 | 22222 | 4:113706536 | 22230 | 4:113862937 |
| 22232 | 4:113865696 | 22233 | 4:113865709 | 22234 | 4:113865711 | 22235 | 4:113889872 | 22236 | 4:113924572 |
| 22239 | 4:113963854 | 22242 | 4:114103350 | 22247 | 4:114278681 | 22261 | 4:114688072 | 22262 | 4:114690368 |
| 22263 | 4:114690442 | 22264 | 4:114691485 | 22265 | 4:114692365 | 22270 | 4:114751599 | 22271 | 4:114758803 |
| 22273 | 4:114770151 | 22275 | 4:114805260 | 22276 | 4:114812473 | 22277 | 4:114812501 | 22278 | 4:114812550 |
| 22279 | 4:114812583 | 22289 | 4:115031057 | 22290 | 4:115033034 | 22291 | 4:115043261 | 22292 | 4:115066503 |
| 22293 | 4:115067836 | 22294 | 4:115071402 | 22295 | 4:115072381 | 22296 | 4:115074258 | 22297 | 4:115075256 |
| 22298 | 4:115077110 | 22299 | 4:115077972 | 22300 | 4:115079711 | 22301 | 4:115105025 | 22304 | 4:115127050 |
| 22305 | 4:115127065 | 22309 | 4:115153886 | 22310 | 4:115153896 | 22311 | 4:115163025 | 22326 | 4:115413253 |
| 22327 | 4:115413261 | 22328 | 4:115413321 | 22329 | 4:115413386 | 22330 | 4:115413395 | 22359 | 4:116192647 |
| 22360 | 4:116192669 | 22361 | 4:116192689 | 22362 | 4:116192738 | 22363 | 4:116192764 | 22379 | 4:116445855 |
| 22402 | 4:116915804 | 22408 | 4:117000567 | 22409 | 4:117000602 | 22410 | 4:117000603 | 22420 | 4:117210404 |
| 22427 | 4:117486610 | 22428 | 4:117505997 | 22448 | 4:117829938 | 22465 | 4:118353876 | 22466 | 4:118358762 |
| 22472 | 4:118419883 | 22476 | 4:118517071 | 22485 | 4:118646844 | 22490 | 4:118756391 | 22494 | 4:118829611 |
| 22496 | 4:118829788 | 22504 | 4:118939017 | 22523 | 4:119457121 | 22535 | 4:119689809 | 22553 | 5:77074 |
| 22579 | 5:827590 | 22580 | 5:834238 | 22581 | 5:882265 | 22582 | 5:882349 | 22583 | 5:882384 |
| 22584 | 5:903638 | 22597 | 5:1099972 | 22608 | 5:1345105 | 22610 | 5:1356051 | 22611 | 5:1356065 |
| 22612 | 5:1356090 | 22613 | 5:1356140 | 22614 | 5:1356177 | 22615 | 5:1356191 | 22618 | 5:1456630 |
| 22623 | 5:1589478 | 22625 | 5:1618964 | 22670 | 5:2514974 | 22681 | 5:2735966 | 22684 | 5:2800666 |
| 22698 | 5:3349859 | 22699 | 5:3349891 | 22719 | 5:4068348 | 22749 | 5:4663849 | 22757 | 5:4889807 |
| 22768 | 5:5162198 | 22770 | 5:5188536 | 22772 | 5:5196871 | 22778 | 5:5266489 | 22780 | 5:5300800 |
| 22789 | 5:5528412 | 22792 | 5:5566952 | 22804 | 5:5799927 | 22805 | 5:5817089 | 22814 | 5:6016548 |
| 22822 | 5:6285112 | 22826 | 5:6336054 | 22828 | 5:6436778 | 22838 | 5:6632102 | 22859 | 5:7063231 |
| 22871 | 5:7268739 | 22873 | 5:7277360 | 22874 | 5:7277411 | 22877 | 5:7377438 | 22895 | 5:7885365 |
| 22915 | 5:8557986 | 22928 | 5:8826886 | 22932 | 5:8934422 | 22933 | 5:8934467 | 22934 | 5:8934474 |
| 22962 | 5:9693605 | 22964 | 5:9730547 | 22967 | 5:9797519 | 22968 | 5:9803049 | 22969 | 5:9856996 |
| 22970 | 5:9857138 | 22971 | 5:9858180 | 22976 | 5:9982908 | 22978 | 5:10011555 | 22980 | 5:10029875 |
| 22981 | 5:10034496 | 22989 | 5:10156211 | 22991 | 5:10201512 | 22992 | 5:10252022 | 22993 | 5:10278854 |
| 23007 | 5:10506388 | 23014 | 5:10670341 | 23020 | 5:10828061 | 23021 | 5:10843507 | 23022 | 5:10843627 |
| 23023 | 5:10844849 | 23031 | 5:11089678 | 23063 | 5:12028271 | 23067 | 5:12102588 | 23068 | 5:12103176 |
| 23073 | 5:12156021 | 23074 | 5:12156678 | 23083 | 5:12325031 | 23084 | 5:12326106 | 23096 | 5:12460715 |
| 23097 | 5:12473390 | 23105 | 5:12528945 | 23111 | 5:12719804 | 23121 | 5:13036017 | 23124 | 5:13069760 |
| 23139 | 5:13541155 | 23151 | 5:14006938 | 23155 | 5:14124180 | 23171 | 5:14599931 | 23172 | 5:14600013 |
| 23175 | 5:14680469 | 23195 | 5:15100766 | 23199 | 5:15173290 | 23216 | 5:15462968 | 23219 | 5:15566954 |
| 23220 | 5:15566955 | 23221 | 5:15566965 | 23222 | 5:15566966 | 23223 | 5:15566972 | 23224 | 5:15566977 |
| 23225 | 5:15567052 | 23226 | 5:15567070 | 23227 | 5:15567096 | 23228 | 5:15567111 | 23232 | 5:15704626 |
| 23266 | 5:16709205 | 23270 | 5:16800463 | 23282 | 5:17119922 | 23285 | 5:17174787 | 23288 | 5:17241349 |
| 23293 | 5:17398558 | 23306 | 5:17895598 | 23307 | 5:17903579 | 23318 | 5:18353350 | 23321 | 5:18410507 |
| 23322 | 5:18410599 | 23338 | 5:18941613 | 23351 | 5:19280880 | 23359 | 5:19568730 | 23374 | 5:19885896 |
| 23376 | 5:19976107 | 23377 | 5:19993960 | 23378 | 5:20014240 | 23381 | 5:20097603 | 23382 | 5:20149313 |
| 23384 | 5:20195300 | 23389 | 5:20436830 | 23396 | 5:20590589 | 23399 | 5:20732321 | 23405 | 5:20832737 |
| 23406 | 5:20832885 | 23407 | 5:20833047 | 23409 | 5:20843567 | 23410 | 5:20910258 | 23411 | 5:20936220 |
| 23416 | 5:21019650 | 23422 | 5:21196220 | 23445 | 5:21745511 | 23447 | 5:21797550 | 23452 | 5:21874789 |
| 23454 | 5:21920920 | 23466 | 5:22115062 | 23473 | 5:22298578 | 23474 | 5:22323880 | 23477 | 5:22412419 |
| 23487 | 5:22513497 | 23491 | 5:22558133 | 23492 | 5:22558154 | 23493 | 5:22558155 | 23502 | 5:22837489 |
| 23503 | 5:22837951 | 23507 | 5:22898017 | 23509 | 5:22944167 | 23510 | 5:22947768 | 23514 | 5:23017065 |
| 23515 | 5:23045588 | 23517 | 5:23055987 | 23524 | 5:23260695 | 23525 | 5:23261205 | 23526 | 5:23269298 |
| 23530 | 5:23386932 | 23533 | 5:23440852 | 23534 | 5:23449942 | 23542 | 5:23608812 | 23545 | 5:23736650 |
| 23548 | 5:23753028 | 23552 | 5:23816911 | 23556 | 5:23949886 | 23561 | 5:23975472 | 23570 | 5:24168834 |
| 23578 | 5:24352659 | 23579 | 5:24352754 | 23587 | 5:24564540 | 23591 | 5:24706081 | 23594 | 5:24861511 |
| 23595 | 5:24861917 | 23596 | 5:24862040 | 23597 | 5:24863117 | 23598 | 5:24864988 | 23599 | 5:24865092 |
| 23610 | 5:25042104 | 23618 | 5:25424142 | 23619 | 5:25424207 | 23622 | 5:25531432 | 23627 | 5:25660326 |
| 23629 | 5:25668094 | 23632 | 5:25770487 | 23635 | 5:25865044 | 23641 | 5:25948717 | 23642 | 5:25953449 |
| 23648 | 5:26274617 | 23652 | 5:26346656 | 23653 | 5:26359865 | 23654 | 5:26372843 | 23655 | 5:26380803 |
| 23661 | 5:26513500 | 23662 | 5:26516803 | 23663 | 5:26527639 | 23664 | 5:26531218 | 23672 | 5:26728680 |
| 23674 | 5:26750096 | 23679 | 5:26881116 | 23684 | 5:27043324 | 23685 | 5:27062257 | 23686 | 5:27098658 |
| 23700 | 5:27397526 | 23705 | 5:27478824 | 23712 | 5:27611131 | 23718 | 5:27732993 | 23724 | 5:27782046 |
| 23726 | 5:27821648 | 23730 | 5:27894859 | 23736 | 5:28067367 | 23738 | 5:28076547 | 23741 | 5:28125774 |
| 23750 | 5:28398456 | 23754 | 5:28468312 | 23755 | 5:28474788 | 23770 | 5:28759211 | 23771 | 5:28764621 |
| 23776 | 5:28916451 | 23779 | 5:28965670 | 23787 | 5:29216390 | 23792 | 5:29377131 | 23809 | 5:29727121 |
| 23815 | 5:29862958 | 23819 | 5:29973188 | 23830 | 5:30143566 | 23838 | 5:30392463 | 23844 | 5:30521361 |
| 23845 | 5:30526943 | 23846 | 5:30564929 | 23847 | 5:30571021 | 23848 | 5:30583097 | 23849 | 5:30584287 |
| 23852 | 5:30656853 | 23861 | 5:30885641 | 23863 | 5:30907588 | 23864 | 5:30945722 | 23869 | 5:31116777 |
| 23890 | 5:31693587 | 23899 | 5:32136613 | 23912 | 5:32401173 | 23913 | 5:32414358 | 23916 | 5:32527988 |
| 23917 | 5:32544707 | 23918 | 5:32549914 | 23921 | 5:32589543 | 23931 | 5:32826960 | 23939 | 5:33070073 |
| 23941 | 5:33080199 | 23951 | 5:33311326 | 23955 | 5:33354800 | 23962 | 5:33573362 | 23971 | 5:33769971 |
| 23972 | 5:33803288 | 23979 | 5:33964757 | 23980 | 5:33974899 | 24008 | 5:34894503 | 24010 | 5:34992883 |
| 24011 | 5:34992887 | 24012 | 5:34992888 | 24014 | 5:35004904 | 24015 | 5:35004932 | 24016 | 5:35039187 |
| 24017 | 5:35055317 | 24018 | 5:35080229 | 24022 | 5:35207804 | 24033 | 5:35577195 | 24036 | 5:35771527 |
| 24066 | 5:36566052 | 24079 | 5:37113142 | 24101 | 5:37695072 | 24102 | 5:37711059 | 24104 | 5:37735001 |
| 24109 | 5:37845630 | 24113 | 5:37881472 | 24119 | 5:37991676 | 24122 | 5:38029636 | 24123 | 5:38033300 |
| 24125 | 5:38058027 | 24127 | 5:38106278 | 24129 | 5:38114225 | 24130 | 5:38116179 | 24131 | 5:38135398 |
| 24134 | 5:38174231 | 24144 | 5:38453061 | 24145 | 5:38483708 | 24146 | 5:38527379 | 24147 | 5:38531595 |
| 24148 | 5:38544831 | 24150 | 5:38593359 | 24163 | 5:39154317 | 24165 | 5:39228037 | 24166 | 5:39257315 |
| 24186 | 5:39750764 | 24202 | 5:40183299 | 24204 | 5:40237324 | 24207 | 5:40321836 | 24208 | 5:40321842 |
| 24209 | 5:40321905 | 24210 | 5:40321940 | 24211 | 5:40322458 | 24212 | 5:40322491 | 24214 | 5:40326831 |
| 24215 | 5:40340341 | 24216 | 5:40342525 | 24227 | 5:40812862 | 24251 | 5:41504706 | 24264 | 5:41958537 |
| 24265 | 5:41958597 | 24266 | 5:41958622 | 24267 | 5:41987705 | 24270 | 5:42145772 | 24282 | 5:42611303 |
| 24291 | 5:42843742 | 24296 | 5:42890909 | 24302 | 5:43044298 | 24305 | 5:43082557 | 24314 | 5:43415290 |
| 24315 | 5:43480306 | 24318 | 5:43506882 | 24324 | 5:43599378 | 24331 | 5:43811761 | 24334 | 5:43863604 |
| 24335 | 5:43864090 | 24336 | 5:43864115 | 24337 | 5:43869544 | 24338 | 5:43875060 | 24339 | 5:43878846 |
| 24340 | 5:43880997 | 24341 | 5:43881099 | 24342 | 5:43883078 | 24343 | 5:43897037 | 24344 | 5:43897095 |
| 24346 | 5:43901763 | 24350 | 5:44040614 | 24351 | 5:44049829 | 24360 | 5:44388064 | 24374 | 5:44804564 |
| 24377 | 5:44906731 | 24384 | 5:45175795 | 24391 | 5:45366260 | 24400 | 5:45549099 | 24403 | 5:45619287 |
| 24414 | 5:45917363 | 24422 | 5:46063715 | 24424 | 5:46103767 | 24425 | 5:46103871 | 24435 | 5:46335844 |
| 24440 | 5:46443847 | 24442 | 5:46516496 | 24483 | 5:47241615 | 24489 | 5:47478077 | 24492 | 5:47556051 |
| 24494 | 5:47596785 | 24507 | 5:48011805 | 24509 | 5:48115271 | 24513 | 5:48219521 | 24521 | 5:48357494 |
| 24546 | 5:49071305 | 24547 | 5:49121969 | 24548 | 5:49128930 | 24551 | 5:49244193 | 24555 | 5:49443860 |
| 24557 | 5:49480432 | 24576 | 5:50003215 | 24577 | 5:50004728 | 24587 | 5:50241038 | 24593 | 5:50468141 |
| 24601 | 5:50729869 | 24604 | 5:50805474 | 24606 | 5:50826119 | 24612 | 5:50987421 | 24613 | 5:50995228 |
| 24615 | 5:51012775 | 24616 | 5:51014179 | 24617 | 5:51026281 | 24618 | 5:51028777 | 24619 | 5:51028823 |
| 24620 | 5:51045771 | 24621 | 5:51046494 | 24622 | 5:51046653 | 24623 | 5:51050849 | 24624 | 5:51055590 |
| 24625 | 5:51056409 | 24626 | 5:51074757 | 24627 | 5:51076137 | 24629 | 5:51106212 | 24630 | 5:51125457 |
| 24633 | 5:51222282 | 24639 | 5:51428229 | 24649 | 5:51662795 | 24651 | 5:51737595 | 24652 | 5:51807509 |
| 24660 | 5:52059878 | 24661 | 5:52059908 | 24662 | 5:52059930 | 24680 | 5:52526329 | 24693 | 5:53179786 |
| 24711 | 5:53766718 | 24712 | 5:53779538 | 24713 | 5:53781567 | 24714 | 5:53786730 | 24717 | 5:53862521 |
| 24723 | 5:54013034 | 24732 | 5:54299758 | 24733 | 5:54301166 | 24734 | 5:54304681 | 24735 | 5:54306088 |
| 24736 | 5:54309409 | 24737 | 5:54324570 | 24738 | 5:54332158 | 24740 | 5:54358159 | 24741 | 5:54367083 |
| 24742 | 5:54370187 | 24743 | 5:54376482 | 24745 | 5:54399560 | 24748 | 5:54511599 | 24749 | 5:54514698 |
| 24751 | 5:54563791 | 24755 | 5:54632888 | 24761 | 5:54765009 | 24785 | 5:55509920 | 24789 | 5:55583411 |
| 24790 | 5:55619423 | 24791 | 5:55634685 | 24792 | 5:55640765 | 24793 | 5:55652450 | 24795 | 5:55670462 |
| 24797 | 5:55696567 | 24798 | 5:55702652 | 24799 | 5:55707061 | 24802 | 5:55873933 | 24803 | 5:55879578 |
| 24805 | 5:55940985 | 24807 | 5:55956661 | 24848 | 5:56720568 | 24849 | 5:56792015 | 24853 | 5:56910201 |
| 24854 | 5:56953231 | 24859 | 5:57100134 | 24864 | 5:57204918 | 24873 | 5:57427084 | 24882 | 5:57667264 |
| 24883 | 5:57672067 | 24896 | 5:58068683 | 24904 | 5:58250121 | 24905 | 5:58285910 | 24915 | 5:58749685 |
| 24923 | 5:59304558 | 24925 | 5:59313596 | 24929 | 5:59364960 | 24930 | 5:59365133 | 24941 | 5:59865035 |
| 24950 | 5:60107106 | 24956 | 5:60233686 | 24958 | 5:60262488 | 24960 | 5:60297442 | 24962 | 5:60326833 |
| 24965 | 5:60380312 | 24971 | 5:60510721 | 24972 | 5:60532648 | 24973 | 5:60543258 | 24975 | 5:60557011 |
| 24977 | 5:60595067 | 24983 | 5:60718806 | 24985 | 5:60758142 | 24986 | 5:60766663 | 24987 | 5:60766858 |
| 24996 | 5:61052215 | 25025 | 5:62076373 | 25033 | 5:62426367 | 25040 | 5:62518142 | 25043 | 5:62636512 |
| 25051 | 5:62933839 | 25052 | 5:62941170 | 25083 | 5:63542114 | 25095 | 5:63850062 | 25104 | 5:64226221 |
| 25106 | 5:64234434 | 25118 | 5:64619628 | 25137 | 5:65171005 | 25138 | 5:65183093 | 25142 | 5:65232220 |
| 25151 | 5:65446380 | 25154 | 5:65535107 | 25155 | 5:65542737 | 25156 | 5:65543322 | 25157 | 5:65543372 |
| 25167 | 5:65703423 | 25168 | 5:65712111 | 25169 | 5:65751283 | 25170 | 5:65752854 | 25172 | 5:65760426 |
| 25173 | 5:65761371 | 25179 | 5:66009583 | 25195 | 5:66518756 | 25225 | 5:67447645 | 25226 | 5:67449152 |
| 25227 | 5:67463576 | 25228 | 5:67465088 | 25229 | 5:67468026 | 25230 | 5:67470940 | 25231 | 5:67473722 |
| 25232 | 5:67475068 | 25233 | 5:67475725 | 25234 | 5:67477065 | 25235 | 5:67481131 | 25237 | 5:67514487 |
| 25238 | 5:67526075 | 25239 | 5:67526864 | 25241 | 5:67555054 | 25242 | 5:67563065 | 25243 | 5:67568345 |
| 25251 | 5:67846005 | 25252 | 5:67848289 | 25253 | 5:67851400 | 25260 | 5:68044942 | 25261 | 5:68062320 |
| 25265 | 5:68180283 | 25267 | 5:68195298 | 25277 | 5:68311976 | 25280 | 5:68341993 | 25282 | 5:68419217 |
| 25284 | 5:68527931 | 25292 | 5:68745462 | 25294 | 5:68847086 | 25305 | 5:68998098 | 25309 | 5:69114492 |
| 25314 | 5:69191792 | 25315 | 5:69211669 | 25318 | 5:69282729 | 25319 | 5:69295982 | 25327 | 5:69507467 |
| 25329 | 5:69534170 | 25330 | 5:69540325 | 25339 | 5:69690113 | 25346 | 5:69807021 | 25354 | 5:70143301 |
| 25369 | 5:70657641 | 25371 | 5:70725053 | 25383 | 5:70904784 | 25385 | 5:70931624 | 25388 | 5:71041333 |
| 25390 | 5:71113115 | 25393 | 5:71147255 | 25397 | 5:71158676 | 25399 | 5:71204940 | 25401 | 5:71215860 |
| 25402 | 5:71247169 | 25404 | 5:71274477 | 25406 | 5:71300632 | 25408 | 5:71346087 | 25409 | 5:71377241 |
| 25418 | 5:71535392 | 25422 | 5:71629712 | 25432 | 5:71856285 | 25433 | 5:71893342 | 25434 | 5:71896515 |
| 25435 | 5:71909707 | 25438 | 5:71958333 | 25442 | 5:72039709 | 25443 | 5:72058094 | 25445 | 5:72064330 |
| 25458 | 5:72293857 | 25461 | 5:72366005 | 25462 | 5:72368884 | 25475 | 5:72696496 | 25500 | 5:73554593 |
| 25504 | 5:73672740 | 25521 | 5:73884415 | 25522 | 5:73908825 | 25523 | 5:73980075 | 25529 | 5:74150522 |
| 25532 | 5:74225942 | 25533 | 5:74245072 | 25545 | 5:74650187 | 25547 | 5:74725927 | 25549 | 5:74742101 |
| 25555 | 5:74908285 | 25556 | 5:74908338 | 25557 | 5:74909826 | 25558 | 5:74911263 | 25559 | 5:74911341 |
| 25560 | 5:74915786 | 25561 | 5:74919175 | 25562 | 5:74925227 | 25563 | 5:74925818 | 25564 | 5:74926028 |
| 25565 | 5:74928887 | 25566 | 5:74936574 | 25571 | 5:75167453 | 25574 | 5:75224410 | 25575 | 5:75224450 |
| 25576 | 5:75224458 | 25577 | 5:75224463 | 25579 | 5:75224524 | 25582 | 5:75261093 | 25586 | 5:75281166 |
| 25599 | 5:75676623 | 25605 | 5:75823353 | 25608 | 5:75912892 | 25609 | 5:75919398 | 25614 | 5:75988734 |
| 25620 | 5:76209127 | 25621 | 5:76269564 | 25622 | 5:76269603 | 25631 | 5:76640050 | 25632 | 5:76666195 |
| 25640 | 5:76784217 | 25649 | 5:77156529 | 25650 | 5:77161120 | 25651 | 5:77180731 | 25652 | 5:77193464 |
| 25653 | 5:77193475 | 25654 | 5:77193500 | 25660 | 5:77277820 | 25661 | 5:77302753 | 25662 | 5:77331990 |
| 25664 | 5:77340074 | 25666 | 5:77362757 | 25667 | 5:77410429 | 25677 | 5:77928157 | 25678 | 5:77928784 |
| 25692 | 5:78324681 | 25698 | 5:78506395 | 25699 | 5:78510349 | 25700 | 5:78522919 | 25708 | 5:78820352 |
| 25709 | 5:78820587 | 25710 | 5:78820588 | 25711 | 5:78820618 | 25712 | 5:78820726 | 25713 | 5:78820795 |
| 25714 | 5:78820814 | 25715 | 5:78821054 | 25718 | 5:78829751 | 25719 | 5:78832107 | 25720 | 5:78832141 |
| 25721 | 5:78832271 | 25752 | 5:79910151 | 25775 | 5:80693563 | 25784 | 5:80982109 | 25788 | 5:81166934 |
| 25794 | 5:81369156 | 25802 | 5:81505086 | 25816 | 5:82023846 | 25818 | 5:82073695 | 25822 | 5:82125171 |
| 25825 | 5:82161343 | 25835 | 5:82321735 | 25839 | 5:82419134 | 25842 | 5:82485421 | 25843 | 5:82485429 |
| 25844 | 5:82491588 | 25847 | 5:82535021 | 25868 | 5:82755673 | 25874 | 5:83018943 | 25879 | 5:83102062 |
| 25885 | 5:83210516 | 25887 | 5:83258916 | 25890 | 5:83347445 | 25919 | 5:83920522 | 25924 | 5:84024864 |
| 26062 | 5:84271561 | 26081 | 5:84723773 | 26087 | 5:84917552 | 26096 | 5:85106613 | 26097 | 5:85112695 |
| 26102 | 5:85286730 | 26138 | 5:86172964 | 26151 | 5:86508471 | 26155 | 5:86538135 | 26167 | 5:86803983 |
| 26168 | 5:86807041 | 26195 | 5:87419158 | 26197 | 5:87436653 | 26205 | 5:87558801 | 26222 | 5:87935293 |
| 26227 | 5:88025590 | 26232 | 5:88169363 | 26240 | 5:88321354 | 26241 | 5:88337164 | 26246 | 5:88393821 |
| 26251 | 5:88442816 | 26257 | 5:88527567 | 26259 | 5:88560515 | 26260 | 5:88562589 | 26264 | 5:88656117 |
| 26265 | 5:88656655 | 26266 | 5:88663005 | 26267 | 5:88664137 | 26268 | 5:88664970 | 26269 | 5:88665685 |
| 26270 | 5:88667764 | 26271 | 5:88668736 | 26272 | 5:88669208 | 26273 | 5:88670166 | 26274 | 5:88670949 |
| 26275 | 5:88671581 | 26276 | 5:88672838 | 26277 | 5:88673518 | 26278 | 5:88675186 | 26279 | 5:88676434 |
| 26280 | 5:88677664 | 26281 | 5:88679714 | 26282 | 5:88681101 | 26289 | 5:88791076 | 26294 | 5:88871429 |
| 26295 | 5:88903163 | 26297 | 5:88927675 | 26303 | 5:89099225 | 26311 | 5:89264046 | 26313 | 5:89283625 |
| 26315 | 5:89343653 | 26316 | 5:89359278 | 26317 | 5:89364312 | 26327 | 5:89668965 | 26337 | 5:89886383 |
| 26338 | 5:89886385 | 26346 | 5:90068114 | 26353 | 5:90321950 | 26354 | 5:90345643 | 26356 | 5:90409538 |
| 26363 | 5:90604091 | 26369 | 5:90678336 | 26375 | 5:90778808 | 26425 | 5:92149419 | 26439 | 5:92379543 |
| 26479 | 5:92796508 | 26480 | 5:92796509 | 26481 | 5:92796641 | 26482 | 5:92796716 | 26486 | 5:92864262 |
| 26490 | 5:92953627 | 26493 | 5:93100450 | 26512 | 5:93520661 | 26515 | 5:93603557 | 26517 | 5:93614794 |
| 26539 | 5:94115151 | 26543 | 5:94190519 | 26550 | 5:94345725 | 26551 | 5:94346916 | 26555 | 5:94480126 |
| 26560 | 5:94697047 | 26567 | 5:94796315 | 26568 | 5:94806990 | 26586 | 5:95221444 | 26588 | 5:95236526 |
| 26590 | 5:95266607 | 26592 | 5:95275712 | 26609 | 5:95732385 | 26613 | 5:95783150 | 26623 | 5:96041063 |
| 26630 | 5:96115730 | 26634 | 5:96229534 | 26636 | 5:96266124 | 26641 | 5:96344421 | 26648 | 5:96479304 |
| 26650 | 5:96555471 | 26653 | 5:96570693 | 26654 | 5:96587114 | 26657 | 5:96683112 | 26658 | 5:96697638 |
| 26659 | 5:96722002 | 26663 | 5:96826963 | 26665 | 5:96891834 | 26666 | 5:96895092 | 26667 | 5:96897342 |
| 26668 | 5:96903857 | 26669 | 5:96904695 | 26670 | 5:96905308 | 26672 | 5:96908444 | 26673 | 5:96909315 |
| 26678 | 5:96986050 | 26682 | 5:97026543 | 26686 | 5:97080334 | 26696 | 5:97322489 | 26701 | 5:97423919 |
| 26702 | 5:97434109 | 26704 | 5:97466504 | 26705 | 5:97487627 | 26710 | 5:97624543 | 26727 | 5:98048878 |
| 26731 | 5:98072135 | 26741 | 5:98284200 | 26743 | 5:98321751 | 26746 | 5:98348925 | 26747 | 5:98360313 |
| 26748 | 5:98369761 | 26753 | 5:98558774 | 26754 | 5:98558789 | 26755 | 5:98558837 | 26756 | 5:98558879 |
| 26757 | 5:98559154 | 26758 | 5:98559190 | 26759 | 5:98559458 | 26760 | 5:98559459 | 26762 | 5:98571338 |
| 26769 | 5:98750219 | 26771 | 5:98819283 | 26774 | 5:98902747 | 26779 | 5:98952191 | 26782 | 5:98988266 |
| 26783 | 5:98991352 | 26786 | 5:99023106 | 26793 | 5:99430794 | 26800 | 5:99597967 | 26801 | 5:99613789 |
| 26803 | 5:99657462 | 26804 | 5:99657533 | 26805 | 5:99657651 | 26806 | 5:99657668 | 26808 | 5:99674708 |
| 26809 | 5:99678222 | 26811 | 5:99698551 | 26812 | 5:99698552 | 26815 | 5:99764163 | 26817 | 5:99773482 |
| 26819 | 5:99784430 | 26827 | 5:99916927 | 26837 | 5:100075903 | 26838 | 5:100085604 | 26839 | 5:100089578 |
| 26841 | 5:100109898 | 26842 | 5:100126206 | 26843 | 5:100133218 | 26846 | 5:100234492 | 26848 | 5:100300261 |
| 26849 | 5:100311514 | 26850 | 5:100341741 | 26852 | 5:100377385 | 26854 | 5:100381117 | 26856 | 5:100389557 |
| 26858 | 5:100426926 | 26866 | 5:100696501 | 26867 | 5:100696607 | 26868 | 5:100698121 | 26869 | 5:100698579 |
| 26872 | 5:100728733 | 26881 | 5:100884953 | 26901 | 5:101256059 | 26903 | 5:101355705 | 26904 | 5:101371243 |
| 26906 | 5:101513339 | 26911 | 5:101595246 | 26915 | 5:101614862 | 26916 | 5:101616220 | 26927 | 5:101872342 |
| 26929 | 5:101932704 | 26930 | 5:101932771 | 26942 | 5:102469703 | 26956 | 5:103101288 | 26957 | 5:103104040 |
| 26959 | 5:103119311 | 26960 | 5:103119357 | 26961 | 5:103119383 | 26962 | 5:103119408 | 26963 | 5:103119413 |
| 26965 | 5:103127016 | 26969 | 5:103180670 | 26970 | 5:103181148 | 26971 | 5:103186080 | 26972 | 5:103186163 |
| 26975 | 5:103229995 | 26976 | 5:103292486 | 26977 | 5:103293544 | 26979 | 5:103324283 | 26987 | 5:103577479 |
| 26992 | 5:103749650 | 26993 | 5:103753540 | 26994 | 5:103765906 | 27009 | 5:104024017 | 27010 | 5:104047698 |
| 27011 | 5:104049926 | 27012 | 5:104051208 | 27014 | 5:104058009 | 27015 | 5:104067878 | 27016 | 5:104090665 |
| 27019 | 5:104166543 | 27022 | 5:104306581 | 27027 | 5:104414866 | 27028 | 5:104421461 | 27029 | 5:104425156 |
| 27030 | 5:104425652 | 27035 | 5:104475838 | 27038 | 5:104487779 | 27048 | 5:104725697 | 27052 | 5:104812593 |
| 27057 | 5:104944974 | 27059 | 5:104962040 | 27065 | 5:105070263 | 27066 | 5:105095351 | 27070 | 5:105138348 |
| 27071 | 5:105144347 | 27088 | 5:105596087 | 27097 | 5:105756724 | 27103 | 5:105773809 | 27107 | 5:105778777 |
| 27108 | 5:105779368 | 27151 | 5:106320490 | 27154 | 5:106351560 | 27160 | 5:106474477 | 27171 | 5:106583604 |
| 27173 | 5:106588115 | 27177 | 5:106701615 | 27184 | 5:106816406 | 27190 | 5:106904200 | 27191 | 5:106904337 |
| 27192 | 5:106904427 | 27193 | 5:106904456 | 27194 | 5:106906152 | 27196 | 5:106921443 | 27198 | 5:106952814 |
| 27199 | 5:106952815 | 27208 | 5:107090339 | 27210 | 5:107152886 | 27211 | 5:107155008 | 27213 | 5:107195685 |
| 27214 | 5:107203743 | 27226 | 5:107421295 | 27230 | 5:107445396 | 27236 | 5:107578561 | 27239 | 5:107683951 |
| 27242 | 5:107725814 | 27258 | 5:108050852 | 27260 | 5:108055831 | 27261 | 5:108055990 | 27264 | 5:108099551 |
| 27266 | 5:108207411 | 27267 | 5:108207896 | 27270 | 5:108230364 | 27272 | 5:108235905 | 27277 | 5:108323867 |
| 27296 | 5:108547597 | 27312 | 5:108942683 | 27314 | 5:108955049 | 27316 | 5:108984906 | 27320 | 5:109034391 |
| 27322 | 5:109075632 | 27323 | 5:109090548 | 27324 | 5:109096526 | 27325 | 5:109153205 | 27327 | 5:109154168 |
| 27328 | 5:109178144 | 27329 | 5:109178358 | 27330 | 5:109179308 | 27335 | 5:109320605 | 27339 | 5:109412747 |
| 27348 | 5:109611685 | 27349 | 5:109615284 | 27352 | 5:109687895 | 27354 | 5:109722487 | 27356 | 5:109749503 |
| 27357 | 5:109754096 | 27361 | 5:109808607 | 27362 | 5:109847158 | 27368 | 5:109988776 | 27369 | 5:109989550 |
| 27371 | 5:110069248 | 27375 | 5:110112327 | 27379 | 5:110193224 | 27383 | 5:110314027 | 27384 | 5:110353226 |
| 27385 | 5:110369778 | 27386 | 5:110373456 | 27387 | 5:110373555 | 27389 | 5:110374859 | 27393 | 5:110398433 |
| 27397 | 5:110509525 | 27400 | 5:110531320 | 27401 | 5:110531367 | 27402 | 5:110532648 | 27403 | 5:110532703 |
| 27404 | 5:110532709 | 27405 | 5:110533852 | 27406 | 5:110533859 | 27407 | 5:110534455 | 27410 | 5:110564870 |
| 27412 | 5:110569021 | 27413 | 5:110569141 | 27414 | 5:110569146 | 27419 | 5:110751512 | 27431 | 5:111053132 |
| 27433 | 5:111063936 | 27434 | 5:111065855 | 27435 | 5:111065875 | 27446 | 5:111321416 | 27452 | 5:111501396 |
| 27453 | 5:111502809 | 27460 | 5:111753901 | 27465 | 5:111845063 | 27468 | 5:111862953 | 27469 | 5:111864267 |
| 27480 | 5:112115889 | 27481 | 5:112115946 | 27482 | 5:112126293 | 27483 | 5:112126298 | 27484 | 5:112133653 |
| 27485 | 5:112135929 | 27489 | 5:112235754 | 27492 | 5:112343380 | 27495 | 5:112408202 | 27498 | 5:112448429 |
| 27499 | 5:112455682 | 27505 | 5:112604562 | 27507 | 5:112634025 | 27508 | 5:112641994 | 27510 | 5:112722034 |
| 27513 | 5:112805426 | 27516 | 5:112917350 | 27527 | 5:113148146 | 27528 | 5:113148464 | 27529 | 5:113149795 |
| 27550 | 5:113769146 | 27556 | 5:113920144 | 27558 | 5:113928017 | 27564 | 5:114059697 | 27573 | 5:114343422 |
| 27574 | 5:114343454 | 27577 | 5:114569421 | 27581 | 5:114723102 | 27590 | 5:115002143 | 27593 | 5:115052949 |
| 27600 | 5:115255504 | 27601 | 5:115265500 | 27608 | 5:115466163 | 27615 | 5:115514965 | 27616 | 5:115517449 |
| 27626 | 5:115682503 | 27629 | 5:115766557 | 27651 | 5:116121476 | 27661 | 5:116390999 | 27663 | 5:116423647 |
| 27665 | 5:116464508 | 27669 | 5:116511553 | 27672 | 5:116537986 | 27674 | 5:116577132 | 27685 | 5:116593122 |
| 27686 | 5:116593191 | 27687 | 5:116593192 | 27689 | 5:116593706 | 27690 | 5:116593715 | 27691 | 5:116593762 |
| 27692 | 5:116593794 | 27693 | 5:116593850 | 27703 | 5:116596428 | 27704 | 5:116596441 | 27705 | 5:116596625 |
| 27709 | 5:116597945 | 27710 | 5:116598050 | 27713 | 5:116600211 | 27714 | 5:116601776 | 27717 | 5:116622560 |
| 27723 | 5:116699001 | 27724 | 5:116699079 | 27725 | 5:116699138 | 27726 | 5:116699145 | 27727 | 5:116699159 |
| 27732 | 5:116743643 | 27734 | 5:116745570 | 27735 | 5:116757705 | 27740 | 5:116908657 | 27741 | 5:116911216 |
| 27742 | 5:116911371 | 27743 | 5:116911376 | 27744 | 5:116915304 | 27745 | 5:116915313 | 27746 | 5:116915402 |
| 27748 | 5:116922121 | 27751 | 5:116973879 | 27753 | 5:116989243 | 27754 | 5:116991259 | 27762 | 5:117097690 |
| 27767 | 5:117234102 | 27769 | 5:117262549 | 27770 | 5:117262555 | 27771 | 5:117262556 | 27773 | 5:117262630 |
| 27785 | 5:117459589 | 27786 | 5:117469696 | 27787 | 5:117471979 | 27788 | 5:117473591 | 27789 | 5:117474224 |
| 27790 | 5:117475215 | 27791 | 5:117476538 | 27799 | 5:117579793 | 27805 | 5:117645005 | 27815 | 5:117785747 |
| 27818 | 5:117850396 | 27844 | 5:118401624 | 27854 | 5:118620070 | 27859 | 5:118696815 | 27875 | 5:118913308 |
| 27879 | 5:118954058 | 27880 | 5:118959454 | 27881 | 5:118971379 | 27882 | 5:118972496 | 27883 | 5:118978705 |
| 27885 | 5:118982714 | 27900 | 5:119181604 | 27915 | 5:119570841 | 27918 | 5:119667915 | 27921 | 5:119771063 |
| 27922 | 5:119774093 | 27923 | 5:119775410 | 27926 | 5:119853084 | 27931 | 5:119945721 | 27932 | 5:119995446 |
| 27964 | 6:844150 | 27965 | 6:844153 | 27972 | 6:1031446 | 27980 | 6:1285962 | 27990 | 6:1702526 |
| 27992 | 6:1761466 | 27993 | 6:1761542 | 27994 | 6:1761563 | 27995 | 6:1774727 | 27998 | 6:1945674 |
| 28030 | 6:2057409 | 28044 | 6:2394123 | 28046 | 6:2394185 | 28047 | 6:2418816 | 28048 | 6:2418834 |
| 28054 | 6:2624789 | 28055 | 6:2625544 | 28058 | 6:2663078 | 28059 | 6:2672214 | 28077 | 6:3108815 |
| 28078 | 6:3108854 | 28087 | 6:3376371 | 28088 | 6:3380776 | 28089 | 6:3383123 | 28090 | 6:3386603 |
| 28091 | 6:3388251 | 28092 | 6:3392342 | 28094 | 6:3398350 | 28095 | 6:3399218 | 28096 | 6:3399858 |
| 28097 | 6:3402165 | 28099 | 6:3423360 | 28100 | 6:3425355 | 28102 | 6:3444538 | 28104 | 6:3449326 |
| 28106 | 6:3469199 | 28110 | 6:3575704 | 28111 | 6:3585145 | 28112 | 6:3587923 | 28113 | 6:3587933 |
| 28114 | 6:3588243 | 28115 | 6:3588261 | 28116 | 6:3589475 | 28117 | 6:3589994 | 28124 | 6:3680340 |
| 28142 | 6:4189994 | 28143 | 6:4197131 | 28147 | 6:4297910 | 28149 | 6:4300166 | 28179 | 6:4998395 |
| 28183 | 6:5135323 | 28188 | 6:5332029 | 28190 | 6:5359913 | 28191 | 6:5363670 | 28192 | 6:5366571 |
| 28193 | 6:5417516 | 28194 | 6:5420554 | 28198 | 6:5638260 | 28208 | 6:5736468 | 28213 | 6:5970478 |
| 28215 | 6:6023720 | 28217 | 6:6068366 | 28218 | 6:6086349 | 28219 | 6:6090367 | 28220 | 6:6099226 |
| 28222 | 6:6135400 | 28223 | 6:6135571 | 28224 | 6:6135977 | 28225 | 6:6142753 | 28226 | 6:6145385 |
| 28227 | 6:6146291 | 28229 | 6:6150122 | 28231 | 6:6213823 | 28233 | 6:6252521 | 28236 | 6:6303620 |
| 28237 | 6:6304251 | 28241 | 6:6327370 | 28246 | 6:6434692 | 28248 | 6:6445175 | 28251 | 6:6505947 |
| 28252 | 6:6508802 | 28253 | 6:6508845 | 28256 | 6:6598639 | 28257 | 6:6630222 | 28259 | 6:6653707 |
| 28260 | 6:6653733 | 28261 | 6:6653820 | 28262 | 6:6654166 | 28263 | 6:6654264 | 28265 | 6:6674156 |
| 28266 | 6:6675058 | 28296 | 6:7438091 | 28297 | 6:7438117 | 28306 | 6:7840060 | 28315 | 6:8178961 |
| 28325 | 6:8498664 | 28330 | 6:8732227 | 28331 | 6:8782400 | 28338 | 6:8951172 | 28340 | 6:8957062 |
| 28344 | 6:8978291 | 28348 | 6:8993893 | 28359 | 6:9166519 | 28360 | 6:9196740 | 28361 | 6:9200024 |
| 28362 | 6:9200854 | 28363 | 6:9201774 | 28364 | 6:9203527 | 28365 | 6:9204459 | 28366 | 6:9205411 |
| 28367 | 6:9207235 | 28368 | 6:9208280 | 28369 | 6:9209206 | 28411 | 6:10509217 | 28417 | 6:10679784 |
| 28436 | 6:11171281 | 28444 | 6:11394489 | 28448 | 6:11465975 | 28461 | 6:11714410 | 28468 | 6:11859669 |
| 28469 | 6:11867154 | 28471 | 6:11887100 | 28489 | 6:12266091 | 28492 | 6:12331408 | 28493 | 6:12406590 |
| 28496 | 6:12449553 | 28499 | 6:12460758 | 28505 | 6:12583026 | 28520 | 6:12951400 | 28523 | 6:13000409 |
| 28524 | 6:13001855 | 28525 | 6:13027415 | 28528 | 6:13118294 | 28531 | 6:13124831 | 28532 | 6:13124859 |
| 28533 | 6:13124885 | 28539 | 6:13212064 | 28550 | 6:13497569 | 28554 | 6:13707108 | 28566 | 6:14075733 |
| 28572 | 6:14295399 | 28592 | 6:14776571 | 28593 | 6:14795242 | 28596 | 6:14878264 | 28598 | 6:14937683 |
| 28614 | 6:15271409 | 28615 | 6:15279031 | 28628 | 6:15511470 | 28631 | 6:15552360 | 28632 | 6:15552836 |
| 28633 | 6:15552929 | 28634 | 6:15554381 | 28635 | 6:15554470 | 28639 | 6:15665547 | 28640 | 6:15665565 |
| 28644 | 6:15724964 | 28659 | 6:16024226 | 28672 | 6:16407876 | 28675 | 6:16429738 | 28676 | 6:16431811 |
| 28680 | 6:16480595 | 28681 | 6:16483148 | 28688 | 6:16631779 | 28690 | 6:16706078 | 28692 | 6:16770941 |
| 28701 | 6:16924337 | 28702 | 6:16926868 | 28704 | 6:16957756 | 28710 | 6:17056311 | 28711 | 6:17056315 |
| 28712 | 6:17080889 | 28734 | 6:17680046 | 28737 | 6:17695292 | 28746 | 6:17927268 | 28747 | 6:17931957 |
| 28748 | 6:17938149 | 28761 | 6:18039717 | 28772 | 6:18212765 | 28777 | 6:18284502 | 28778 | 6:18314365 |
| 28789 | 6:18646895 | 28790 | 6:18701849 | 28791 | 6:18731749 | 28799 | 6:18864835 | 28807 | 6:19042586 |
| 28809 | 6:19074411 | 28811 | 6:19074485 | 28815 | 6:19190465 | 28826 | 6:19438867 | 28827 | 6:19439848 |
| 28829 | 6:19456967 | 28830 | 6:19462913 | 28831 | 6:19495081 | 28839 | 6:19649377 | 28848 | 6:19872402 |
| 28850 | 6:19894311 | 28851 | 6:19923403 | 28858 | 6:20135992 | 28860 | 6:20184822 | 28861 | 6:20192872 |
| 28862 | 6:20206522 | 28872 | 6:20397821 | 28876 | 6:20495916 | 28881 | 6:20600966 | 28882 | 6:20600990 |
| 28907 | 6:21325769 | 28908 | 6:21355207 | 28910 | 6:21392641 | 28913 | 6:21439632 | 28914 | 6:21439691 |
| 28915 | 6:21439763 | 28921 | 6:21659954 | 28922 | 6:21672266 | 28923 | 6:21678873 | 28925 | 6:21686069 |
| 28926 | 6:21691325 | 28927 | 6:21703290 | 28928 | 6:21714263 | 28933 | 6:21771563 | 28934 | 6:21772453 |
| 28935 | 6:21777544 | 28939 | 6:21951701 | 28942 | 6:22033622 | 28962 | 6:22379732 | 28964 | 6:22422233 |
| 28967 | 6:22500214 | 28968 | 6:22500233 | 28991 | 6:23037086 | 28997 | 6:23243928 | 29000 | 6:23295269 |
| 29005 | 6:23444102 | 29006 | 6:23457488 | 29010 | 6:23533175 | 29013 | 6:23555860 | 29015 | 6:23571729 |
| 29024 | 6:23873913 | 29029 | 6:23921917 | 29047 | 6:24373068 | 29051 | 6:24503230 | 29053 | 6:24518463 |
| 29064 | 6:24837558 | 29070 | 6:24942515 | 29071 | 6:24948898 | 29076 | 6:25007714 | 29077 | 6:25024841 |
| 29078 | 6:25039222 | 29083 | 6:25164226 | 29095 | 6:25528726 | 29100 | 6:25695588 | 29102 | 6:25788477 |
| 29108 | 6:25887098 | 29117 | 6:26022919 | 29118 | 6:26033719 | 29127 | 6:26200013 | 29129 | 6:26282778 |
| 29130 | 6:26290561 | 29139 | 6:26655531 | 29140 | 6:26655687 | 29142 | 6:26679658 | 29163 | 6:27215526 |
| 29167 | 6:27355784 | 29174 | 6:27472557 | 29186 | 6:27864310 | 29212 | 6:28647979 | 29231 | 6:29128064 |
| 29237 | 6:29212166 | 29239 | 6:29328156 | 29240 | 6:29328334 | 29241 | 6:29328350 | 29243 | 6:29376641 |
| 29245 | 6:29389036 | 29247 | 6:29435290 | 29253 | 6:29483476 | 29259 | 6:29565346 | 29265 | 6:29684500 |
| 29279 | 6:30075635 | 29342 | 6:30145040 | 29365 | 6:30563336 | 29399 | 6:31576983 | 29412 | 6:31922816 |
| 29421 | 6:32195477 | 29425 | 6:32453198 | 29426 | 6:32493429 | 29468 | 6:34036502 | 29472 | 6:34067879 |
| 29473 | 6:34068521 | 29486 | 6:34217305 | 29587 | 6:34344002 | 29660 | 6:34467878 | 29733 | 6:34605510 |
| 29954 | 6:35510819 | 29957 | 6:35561016 | 29986 | 6:35857940 | 30022 | 6:36053886 | 30024 | 6:36074911 |
| 30130 | 6:36395104 | 30139 | 6:36431315 | 30145 | 6:36444505 | 30148 | 6:36448732 | 30174 | 6:36489959 |
| 30250 | 6:36553066 | 30255 | 6:36561452 | 30300 | 6:36642083 | 30317 | 6:36688082 | 30321 | 6:36706370 |
| 30383 | 6:36857123 | 30808 | 6:37218423 | 31143 | 6:37396789 | 31963 | 6:39186781 | 31964 | 6:39249570 |
| 31972 | 6:39371669 | 31973 | 6:39392848 | 31997 | 6:39755061 | 32033 | 6:40227846 | 32038 | 6:40262465 |
| 32095 | 6:40813933 | 32099 | 6:40846711 | 32101 | 6:40869544 | 32162 | 6:41204988 | 32163 | 6:41205018 |
| 32194 | 6:41998863 | 32195 | 6:42004652 | 32196 | 6:42005216 | 32197 | 6:42010829 | 32198 | 6:42011964 |
| 32199 | 6:42014380 | 32200 | 6:42017579 | 32202 | 6:42030911 | 32206 | 6:42098249 | 32238 | 6:42816146 |
| 32247 | 6:42950720 | 32272 | 6:43082880 | 32273 | 6:43090582 | 32274 | 6:43121398 | 32288 | 6:43377340 |
| 32289 | 6:43378488 | 32310 | 6:43688837 | 32324 | 6:43887416 | 32334 | 6:44040805 | 32335 | 6:44059941 |
| 32336 | 6:44072335 | 32349 | 6:44361206 | 32359 | 6:44628166 | 32367 | 6:44692016 | 32483 | 6:45118837 |
| 32493 | 6:45218126 | 32498 | 6:45277318 | 32501 | 6:45322457 | 32503 | 6:45335602 | 32504 | 6:45364598 |
| 32507 | 6:45390455 | 32509 | 6:45416952 | 32513 | 6:45505416 | 32514 | 6:45522021 | 32520 | 6:45688164 |
| 32531 | 6:45935668 | 32532 | 6:45982939 | 32534 | 6:46022376 | 32535 | 6:46078755 | 32545 | 6:46209779 |
| 32550 | 6:46337526 | 32552 | 6:46378480 | 32553 | 6:46389387 | 32554 | 6:46390042 | 32577 | 6:46954860 |
| 32582 | 6:47076276 | 32595 | 6:47353083 | 32606 | 6:47640345 | 32616 | 6:47866852 | 32618 | 6:47903812 |
| 32622 | 6:48018241 | 32634 | 6:48398260 | 32642 | 6:48750204 | 32664 | 6:49330544 | 32666 | 6:49366546 |
| 32681 | 6:49731100 | 32682 | 6:49732769 | 32683 | 6:49734879 | 32684 | 6:49738480 | 32685 | 6:49759413 |
| 32686 | 6:49766766 | 32687 | 6:49773026 | 32690 | 6:49804879 | 32691 | 6:49816719 | 32697 | 6:49895642 |
| 32705 | 6:50190150 | 32709 | 6:50252554 | 32711 | 6:50316194 | 32712 | 6:50316384 | 32713 | 6:50316707 |
| 32714 | 6:50342873 | 32745 | 6:51287866 | 32761 | 6:51779722 | 32770 | 6:52097852 | 32771 | 6:52097941 |
| 32776 | 6:52230394 | 32777 | 6:52230455 | 32779 | 6:52258121 | 32811 | 6:52492036 | 32823 | 6:52776675 |
| 32839 | 6:53189333 | 32841 | 6:53215914 | 32845 | 6:53260563 | 32856 | 6:53542273 | 32858 | 6:53587223 |
| 32871 | 6:53982975 | 32882 | 6:54231133 | 32895 | 6:54705767 | 32905 | 6:55072307 | 32957 | 6:55697219 |
| 32965 | 6:55964167 | 32981 | 6:56569534 | 32982 | 6:56580948 | 32997 | 6:56927864 | 32998 | 6:56927881 |
| 32999 | 6:56927920 | 33000 | 6:56928100 | 33011 | 6:57258298 | 33013 | 6:57310043 | 33016 | 6:57383139 |
| 33019 | 6:57452741 | 33021 | 6:57538068 | 33024 | 6:57589915 | 33053 | 6:58398195 | 33055 | 6:58507178 |
| 33065 | 6:58716078 | 33075 | 6:59092959 | 33080 | 6:59188156 | 33085 | 6:59262762 | 33100 | 6:59749739 |
| 33108 | 6:59853909 | 33118 | 6:59966318 | 33123 | 6:60097949 | 33128 | 6:60226786 | 33130 | 6:60265173 |
| 33131 | 6:60301627 | 33133 | 6:60345603 | 33135 | 6:60373758 | 33138 | 6:60422003 | 33140 | 6:60468668 |
| 33152 | 6:60773644 | 33166 | 6:61158905 | 33167 | 6:61161272 | 33172 | 6:61280689 | 33177 | 6:61397201 |
| 33179 | 6:61410508 | 33180 | 6:61421440 | 33187 | 6:61548278 | 33188 | 6:61582228 | 33189 | 6:61589076 |
| 33190 | 6:61673425 | 33217 | 6:62603953 | 33226 | 6:62889998 | 33231 | 6:62988117 | 33232 | 6:63010364 |
| 33233 | 6:63010380 | 33235 | 6:63029593 | 33237 | 6:63097017 | 33247 | 6:63307538 | 33248 | 6:63308671 |
| 33249 | 6:63347756 | 33269 | 6:64151594 | 33281 | 6:64662329 | 33282 | 6:64665326 | 33293 | 6:65016085 |
| 33300 | 6:65345032 | 33302 | 6:65383049 | 33304 | 6:65420404 | 33305 | 6:65422497 | 33306 | 6:65422592 |
| 33307 | 6:65456807 | 33325 | 6:66109765 | 33332 | 6:66343250 | 33334 | 6:66387388 | 33340 | 6:66651619 |
| 33345 | 6:66831939 | 33348 | 6:66876573 | 33353 | 6:66988792 | 33356 | 6:67035579 | 33358 | 6:67089021 |
| 33373 | 6:67582129 | 33382 | 6:68017233 | 33387 | 6:68177405 | 33390 | 6:68268998 | 33395 | 6:68352179 |
| 33401 | 6:68565298 | 33402 | 6:68589087 | 33403 | 6:68598341 | 33410 | 6:68728215 | 33413 | 6:68763773 |
| 33415 | 6:68828972 | 33417 | 6:68885563 | 33420 | 6:68935879 | 33426 | 6:69138647 | 33428 | 6:69168309 |
| 33430 | 6:69216815 | 33434 | 6:69287580 | 33436 | 6:69352586 | 33439 | 6:69439499 | 33445 | 6:69755083 |
| 33452 | 6:69895326 | 33464 | 6:70383634 | 33465 | 6:70399966 | 33466 | 6:70404486 | 33467 | 6:70405959 |
| 33468 | 6:70423245 | 33472 | 6:70564450 | 33481 | 6:70877482 | 33482 | 6:70900974 | 33490 | 6:71126939 |
| 33494 | 6:71294876 | 33504 | 6:71562156 | 33510 | 6:71703879 | 33518 | 6:72060315 | 33522 | 6:72174384 |
| 33531 | 6:72351300 | 33533 | 6:72379846 | 33540 | 6:72542846 | 33548 | 6:72783452 | 33558 | 6:73223485 |
| 33565 | 6:73414475 | 33567 | 6:73481332 | 33570 | 6:73620191 | 33577 | 6:73801067 | 33631 | 6:75798192 |
| 33645 | 6:76545846 | 33646 | 6:76546983 | 33647 | 6:76547862 | 33665 | 6:76855629 | 33677 | 6:77240842 |
| 33680 | 6:77349471 | 33691 | 6:77688509 | 33694 | 6:77787079 | 33711 | 6:78425706 | 33712 | 6:78429257 |
| 33717 | 6:78761621 | 33718 | 6:78761714 | 33721 | 6:78917750 | 33743 | 6:79765417 | 33765 | 6:80471442 |
| 33766 | 6:80482749 | 33767 | 6:80484712 | 33768 | 6:80491703 | 33769 | 6:80530130 | 33770 | 6:80567928 |
| 33771 | 6:80590178 | 33773 | 6:80675168 | 33788 | 6:81051057 | 33814 | 6:82117911 | 33855 | 6:83277672 |
| 33858 | 6:83308141 | 33861 | 6:83347263 | 33864 | 6:83451575 | 33865 | 6:83451628 | 33866 | 6:83451712 |
| 33867 | 6:83451781 | 33868 | 6:83451862 | 33870 | 6:83469477 | 33871 | 6:83470850 | 33872 | 6:83475021 |
| 33884 | 6:83758356 | 33885 | 6:83763398 | 33892 | 6:83946894 | 33893 | 6:83948279 | 33894 | 6:83950501 |
| 33898 | 6:83985933 | 33902 | 6:84087846 | 33903 | 6:84138032 | 33910 | 6:84497175 | 33911 | 6:84497631 |
| 33912 | 6:84497709 | 33913 | 6:84511587 | 33914 | 6:84511656 | 33915 | 6:84513009 | 33932 | 6:84975132 |
| 33933 | 6:85067620 | 33934 | 6:85092477 | 33935 | 6:85092499 | 33936 | 6:85092523 | 33937 | 6:85093071 |
| 33938 | 6:85108153 | 33939 | 6:85108165 | 33940 | 6:85108432 | 33941 | 6:85108773 | 33943 | 6:85136420 |
| 33944 | 6:85137180 | 33950 | 6:85315912 | 33977 | 6:85896040 | 33978 | 6:85896097 | 33979 | 6:85896105 |
| 33980 | 6:85896139 | 33981 | 6:85906390 | 33982 | 6:85908497 | 34003 | 6:86508590 | 34012 | 6:86710155 |
| 34028 | 6:86901499 | 34032 | 6:86989414 | 34034 | 6:87007077 | 34040 | 6:87076603 | 34043 | 6:87111828 |
| 34057 | 6:87463348 | 34067 | 6:87603473 | 34074 | 6:87731662 | 34082 | 6:87796536 | 34085 | 6:87923433 |
| 34086 | 6:87923536 | 34092 | 6:88075544 | 34096 | 6:88164435 | 34097 | 6:88184411 | 34098 | 6:88184633 |
| 34102 | 6:88318314 | 34108 | 6:88486850 | 34116 | 6:88859149 | 34130 | 6:89152164 | 34131 | 6:89181036 |
| 34150 | 6:89663234 | 34152 | 6:89663319 | 34153 | 6:89663348 | 34154 | 6:89674618 | 34162 | 6:89768711 |
| 34170 | 6:89975431 | 34182 | 6:90390426 | 34185 | 6:90416932 | 34186 | 6:90457703 | 34196 | 6:90680946 |
| 34199 | 6:90714640 | 34200 | 6:90726127 | 34206 | 6:90794035 | 34208 | 6:90816980 | 34210 | 6:90823124 |
| 34211 | 6:90826546 | 34212 | 6:90826598 | 34213 | 6:90827159 | 34214 | 6:90836066 | 34215 | 6:90836072 |
| 34216 | 6:90836166 | 34221 | 6:90946704 | 34228 | 6:91105837 | 34229 | 6:91122456 | 34236 | 6:91204532 |
| 34237 | 6:91223593 | 34238 | 6:91226875 | 34239 | 6:91246239 | 34240 | 6:91275706 | 34241 | 6:91282834 |
| 34242 | 6:91284101 | 34243 | 6:91284414 | 34244 | 6:91285011 | 34245 | 6:91285694 | 34246 | 6:91285851 |
| 34247 | 6:91285925 | 34248 | 6:91290335 | 34249 | 6:91294383 | 34250 | 6:91322998 | 34257 | 6:91406353 |
| 34258 | 6:91408596 | 34260 | 6:91433962 | 34261 | 6:91462732 | 34271 | 6:91615288 | 34272 | 6:91616087 |
| 34275 | 6:91649638 | 34277 | 6:91692231 | 34280 | 6:91736404 | 34282 | 6:91751612 | 34284 | 6:91836628 |
| 34287 | 6:91878223 | 34291 | 6:91994872 | 34300 | 6:92336028 | 34302 | 6:92383295 | 34308 | 6:92595572 |
| 34310 | 6:92678382 | 34313 | 6:92774897 | 34317 | 6:92832688 | 34335 | 6:93291707 | 34336 | 6:93291712 |
| 34337 | 6:93291776 | 34343 | 6:93354640 | 34344 | 6:93363500 | 34351 | 6:93496619 | 34353 | 6:93533733 |
| 34361 | 6:93646233 | 34373 | 6:93947051 | 34378 | 6:94069815 | 34389 | 6:94150648 | 34392 | 6:94188032 |
| 34393 | 6:94201168 | 34394 | 6:94204543 | 34395 | 6:94215369 | 34401 | 6:94394468 | 34403 | 6:94416755 |
| 34413 | 6:94854202 | 34424 | 6:95339487 | 34426 | 6:95406474 | 34427 | 6:95406527 | 34429 | 6:95406556 |
| 34432 | 6:95553456 | 34441 | 6:95787499 | 34444 | 6:95884565 | 34449 | 6:96032153 | 34451 | 6:96087038 |
| 34462 | 6:96247005 | 34474 | 6:96452752 | 34496 | 6:97018222 | 34504 | 6:97179175 | 34505 | 6:97179176 |
| 34506 | 6:97179179 | 34507 | 6:97179203 | 34508 | 6:97179214 | 34509 | 6:97179248 | 34510 | 6:97217522 |
| 34511 | 6:97226901 | 34512 | 6:97244565 | 34513 | 6:97244652 | 34514 | 6:97244662 | 34515 | 6:97255320 |
| 34516 | 6:97255455 | 34517 | 6:97255461 | 34528 | 6:97447881 | 34529 | 6:97449063 | 34530 | 6:97453689 |
| 34544 | 6:97650137 | 34547 | 6:97742989 | 34556 | 6:98047323 | 34558 | 6:98052606 | 34566 | 6:98256336 |
| 34570 | 6:98334890 | 34571 | 6:98337762 | 34572 | 6:98337909 | 34573 | 6:98337919 | 34574 | 6:98338489 |
| 34575 | 6:98338596 | 34576 | 6:98338640 | 34577 | 6:98338644 | 34579 | 6:98372826 | 34581 | 6:98379884 |
| 34596 | 6:98671599 | 34608 | 6:98974175 | 34628 | 6:99529167 | 34637 | 6:99783201 | 34641 | 6:99954538 |
| 34669 | 6:100390632 | 34671 | 6:100431779 | 34678 | 6:100585876 | 34685 | 6:100727406 | 34689 | 6:100852199 |
| 34693 | 6:100953221 | 34706 | 6:101294567 | 34707 | 6:101294597 | 34714 | 6:101450871 | 34729 | 6:101787815 |
| 34730 | 6:101837470 | 34739 | 6:101917014 | 34740 | 6:101935968 | 34741 | 6:101941367 | 34744 | 6:101973857 |
| 34754 | 6:102148393 | 34755 | 6:102148443 | 34760 | 6:102234263 | 34766 | 6:102424852 | 34772 | 6:102510343 |
| 34779 | 6:102606444 | 34783 | 6:102645168 | 34784 | 6:102645192 | 34785 | 6:102645198 | 34788 | 6:102756289 |
| 34791 | 6:102773226 | 34793 | 6:102854233 | 34795 | 6:102855258 | 34796 | 6:102857313 | 34797 | 6:102870507 |
| 34798 | 6:102871524 | 34799 | 6:102872128 | 34800 | 6:102873595 | 34801 | 6:102875781 | 34802 | 6:102889726 |
| 34803 | 6:102890450 | 34804 | 6:102892630 | 34805 | 6:102896127 | 34806 | 6:102896985 | 34807 | 6:102927406 |
| 34812 | 6:103013214 | 34822 | 6:103212156 | 34835 | 6:103357734 | 34837 | 6:103396597 | 34839 | 6:103431771 |
| 34841 | 6:103480885 | 34842 | 6:103490310 | 34843 | 6:103490339 | 34844 | 6:103490470 | 34848 | 6:103557818 |
| 34851 | 6:103637428 | 34852 | 6:103640406 | 34854 | 6:103654616 | 34855 | 6:103663732 | 34856 | 6:103741666 |
| 34857 | 6:103773464 | 34858 | 6:103774096 | 34862 | 6:103862052 | 34864 | 6:103898267 | 34876 | 6:104267666 |
| 34881 | 6:104339504 | 34892 | 6:104657713 | 34893 | 6:104658492 | 34897 | 6:104793010 | 34898 | 6:104793414 |
| 34899 | 6:104809492 | 34900 | 6:104809647 | 34901 | 6:104824268 | 34903 | 6:104868243 | 34905 | 6:104881408 |
| 34909 | 6:104928796 | 34914 | 6:105104413 | 34924 | 6:105290637 | 34927 | 6:105378109 | 34929 | 6:105423924 |
| 34931 | 6:105504062 | 34940 | 6:105638984 | 34941 | 6:105650631 | 34950 | 6:105886896 | 34959 | 6:106123065 |
| 34966 | 6:106312210 | 34971 | 6:106540140 | 34979 | 6:106739419 | 34986 | 6:106953368 | 35009 | 6:107508591 |
| 35024 | 6:108020924 | 35045 | 6:108596491 | 35055 | 6:108951951 | 35057 | 6:108952102 | 35059 | 6:108993006 |
| 35068 | 6:109199682 | 35071 | 6:109282471 | 35072 | 6:109282486 | 35102 | 6:109823696 | 35111 | 6:110018683 |
| 35142 | 6:110852081 | 35156 | 6:111034643 | 35157 | 6:111034669 | 35160 | 6:111043273 | 35167 | 6:111198616 |
| 35176 | 6:111399485 | 35180 | 6:111474948 | 35189 | 6:111673792 | 35198 | 6:111776075 | 35200 | 6:111795733 |
| 35201 | 6:111796366 | 35205 | 6:111879814 | 35206 | 6:111880060 | 35208 | 6:111944736 | 35209 | 6:111951572 |
| 35226 | 6:112530579 | 35231 | 6:112650810 | 35234 | 6:112672985 | 35239 | 6:112735477 | 35241 | 6:112750366 |
| 35247 | 6:112863762 | 35251 | 6:112908010 | 35255 | 6:112989544 | 35262 | 6:113261157 | 35272 | 6:113442820 |
| 35276 | 6:113471367 | 35277 | 6:113480954 | 35281 | 6:113591450 | 35288 | 6:113702935 | 35290 | 6:113754589 |
| 35291 | 6:113756287 | 35292 | 6:113756982 | 35293 | 6:113765934 | 35295 | 6:113775951 | 35297 | 6:113799562 |
| 35298 | 6:113821699 | 35300 | 6:113827124 | 35301 | 6:113830450 | 35309 | 6:113999468 | 35311 | 6:114031327 |
| 35312 | 6:114040384 | 35320 | 6:114163874 | 35324 | 6:114205337 | 35327 | 6:114271753 | 35334 | 6:114428994 |
| 35336 | 6:114452241 | 35337 | 6:114471056 | 35342 | 6:114583005 | 35344 | 6:114601159 | 35345 | 6:114602046 |
| 35351 | 6:114719008 | 35355 | 6:114797024 | 35365 | 6:115032289 | 35366 | 6:115035590 | 35367 | 6:115035592 |
| 35376 | 6:115201677 | 35382 | 6:115369888 | 35401 | 6:115856852 | 35403 | 6:115872479 | 35404 | 6:115959886 |
| 35408 | 6:116004094 | 35409 | 6:116011104 | 35416 | 6:116154808 | 35417 | 6:116160046 | 35420 | 6:116200574 |
| 35422 | 6:116286341 | 35425 | 6:116319165 | 35430 | 6:116485262 | 35431 | 6:116485283 | 35432 | 6:116485335 |
| 35433 | 6:116485360 | 35434 | 6:116485372 | 35435 | 6:116485909 | 35436 | 6:116487900 | 35437 | 6:116487922 |
| 35438 | 6:116488922 | 35439 | 6:116489686 | 35440 | 6:116489801 | 35441 | 6:116500010 | 35442 | 6:116500063 |
| 35443 | 6:116500364 | 35444 | 6:116500379 | 35445 | 6:116509870 | 35446 | 6:116514508 | 35447 | 6:116514529 |
| 35453 | 6:116932235 | 35457 | 6:117212852 | 35458 | 6:117247186 | 35459 | 6:117253860 | 35462 | 6:117399949 |
| 35463 | 6:117405411 | 35464 | 6:117444202 | 35465 | 6:117445801 | 35466 | 6:117445935 | 35467 | 6:117463206 |
| 35490 | 7:516102 | 35502 | 7:810744 | 35503 | 7:822079 | 35533 | 7:1434030 | 35534 | 7:1439391 |
| 35535 | 7:1444418 | 35537 | 7:1508351 | 35539 | 7:1552952 | 35546 | 7:1663078 | 35547 | 7:1663134 |
| 35550 | 7:1706720 | 35553 | 7:1757821 | 35560 | 7:1977089 | 35562 | 7:1996339 | 35563 | 7:2036084 |
| 35567 | 7:2112952 | 35577 | 7:2352038 | 35587 | 7:2461961 | 35590 | 7:2556375 | 35594 | 7:2628007 |
| 35595 | 7:2628008 | 35596 | 7:2628014 | 35597 | 7:2628018 | 35598 | 7:2628066 | 35599 | 7:2628069 |
| 35600 | 7:2628233 | 35601 | 7:2628255 | 35602 | 7:2628256 | 35604 | 7:2630943 | 35605 | 7:2630947 |
| 35606 | 7:2631054 | 35607 | 7:2631064 | 35610 | 7:2682673 | 35611 | 7:2682716 | 35612 | 7:2682743 |
| 35613 | 7:2682770 | 35614 | 7:2682781 | 35615 | 7:2682785 | 35618 | 7:2721521 | 35619 | 7:2728053 |
| 35622 | 7:2823155 | 35624 | 7:2880427 | 35629 | 7:2957250 | 35631 | 7:3004323 | 35641 | 7:3142936 |
| 35643 | 7:3239293 | 35670 | 7:3679709 | 35671 | 7:3679809 | 35672 | 7:3686101 | 35673 | 7:3687496 |
| 35683 | 7:3876358 | 35687 | 7:3947888 | 35688 | 7:3949113 | 35689 | 7:3953770 | 35690 | 7:3965550 |
| 35692 | 7:4018660 | 35693 | 7:4057873 | 35700 | 7:4183821 | 35702 | 7:4203677 | 35704 | 7:4226758 |
| 35706 | 7:4321618 | 35708 | 7:4425609 | 35709 | 7:4432045 | 35713 | 7:4509941 | 35714 | 7:4530497 |
| 35715 | 7:4550094 | 35724 | 7:4890795 | 35725 | 7:4931147 | 35733 | 7:5056411 | 35734 | 7:5076528 |
| 35735 | 7:5077856 | 35736 | 7:5077899 | 35737 | 7:5077986 | 35738 | 7:5078073 | 35743 | 7:5135884 |
| 35744 | 7:5135971 | 35747 | 7:5227598 | 35758 | 7:5539362 | 35759 | 7:5540415 | 35760 | 7:5541785 |
| 35761 | 7:5542691 | 35762 | 7:5543242 | 35763 | 7:5543903 | 35764 | 7:5544868 | 35765 | 7:5545851 |
| 35766 | 7:5546627 | 35767 | 7:5547659 | 35768 | 7:5548421 | 35769 | 7:5554183 | 35770 | 7:5554705 |
| 35771 | 7:5555278 | 35772 | 7:5556329 | 35773 | 7:5560431 | 35775 | 7:5587078 | 35781 | 7:5670138 |
| 35783 | 7:5725687 | 35784 | 7:5728200 | 35787 | 7:5774079 | 35788 | 7:5775685 | 35789 | 7:5775738 |
| 35790 | 7:5775833 | 35791 | 7:5775864 | 35794 | 7:5854438 | 35795 | 7:5867893 | 35796 | 7:5869641 |
| 35797 | 7:5875629 | 35798 | 7:5876835 | 35799 | 7:5879220 | 35800 | 7:5884897 | 35801 | 7:5888476 |
| 35803 | 7:5895045 | 35804 | 7:5905064 | 35807 | 7:5948030 | 35839 | 7:6133269 | 35848 | 7:6256347 |
| 35850 | 7:6276752 | 35852 | 7:6318895 | 35853 | 7:6318915 | 35867 | 7:6376467 | 35868 | 7:6389582 |
| 35877 | 7:6654578 | 35885 | 7:6739561 | 35892 | 7:6820748 | 35896 | 7:6837253 | 35905 | 7:7044892 |
| 35906 | 7:7044893 | 35909 | 7:7081346 | 35912 | 7:7131477 | 35913 | 7:7131577 | 35979 | 7:9765475 |
| 35998 | 7:10641237 | 36009 | 7:11092130 | 36015 | 7:11145935 | 36016 | 7:11154819 | 36019 | 7:11233180 |
| 36020 | 7:11233207 | 36021 | 7:11237049 | 36022 | 7:11296724 | 36023 | 7:11306644 | 36024 | 7:11332158 |
| 36026 | 7:11394630 | 36027 | 7:11394736 | 36028 | 7:11403649 | 36042 | 7:11870658 | 36043 | 7:11885250 |
| 36062 | 7:12227428 | 36070 | 7:12344743 | 36071 | 7:12344879 | 36082 | 7:12522109 | 36088 | 7:12587487 |
| 36093 | 7:12738904 | 36096 | 7:12762542 | 36097 | 7:12763942 | 36099 | 7:12806635 | 36100 | 7:12816543 |
| 36102 | 7:12824241 | 36103 | 7:12852855 | 36106 | 7:12896445 | 36107 | 7:12897971 | 36108 | 7:12898039 |
| 36109 | 7:12898062 | 36111 | 7:12974218 | 36112 | 7:12974361 | 36115 | 7:13016195 | 36121 | 7:13189985 |
| 36122 | 7:13201701 | 36123 | 7:13204223 | 36124 | 7:13204306 | 36125 | 7:13209001 | 36126 | 7:13209008 |
| 36127 | 7:13209051 | 36128 | 7:13209093 | 36129 | 7:13211564 | 36130 | 7:13211698 | 36131 | 7:13211746 |
| 36133 | 7:13234671 | 36135 | 7:13241201 | 36136 | 7:13248867 | 36137 | 7:13252997 | 36145 | 7:13436247 |
| 36160 | 7:13763740 | 36161 | 7:13764174 | 36163 | 7:13826339 | 36164 | 7:13826607 | 36165 | 7:13847719 |
| 36166 | 7:13847770 | 36167 | 7:13848168 | 36169 | 7:13907560 | 36170 | 7:13907747 | 36171 | 7:13908190 |
| 36175 | 7:13970540 | 36176 | 7:13970551 | 36177 | 7:13971039 | 36182 | 7:14105363 | 36187 | 7:14216180 |
| 36188 | 7:14278452 | 36196 | 7:14568342 | 36197 | 7:14569870 | 36198 | 7:14569910 | 36199 | 7:14570835 |
| 36200 | 7:14571116 | 36201 | 7:14571263 | 36202 | 7:14572010 | 36203 | 7:14572312 | 36204 | 7:14574790 |
| 36205 | 7:14574794 | 36206 | 7:14574824 | 36207 | 7:14574906 | 36208 | 7:14575332 | 36209 | 7:14575373 |
| 36210 | 7:14576408 | 36211 | 7:14576429 | 36212 | 7:14576444 | 36213 | 7:14577168 | 36214 | 7:14577625 |
| 36215 | 7:14578417 | 36216 | 7:14578461 | 36217 | 7:14578495 | 36218 | 7:14587075 | 36221 | 7:14674502 |
| 36222 | 7:14674589 | 36224 | 7:14688808 | 36225 | 7:14689037 | 36226 | 7:14690346 | 36227 | 7:14690813 |
| 36228 | 7:14692758 | 36229 | 7:14695086 | 36230 | 7:14790790 | 36231 | 7:14846055 | 36232 | 7:14847442 |
| 36241 | 7:15055421 | 36242 | 7:15055460 | 36246 | 7:15113333 | 36247 | 7:15116085 | 36248 | 7:15116605 |
| 36249 | 7:15120550 | 36250 | 7:15121347 | 36251 | 7:15121459 | 36252 | 7:15131143 | 36254 | 7:15149591 |
| 36255 | 7:15158037 | 36257 | 7:15188314 | 36258 | 7:15219440 | 36262 | 7:15240130 | 36267 | 7:15342910 |
| 36269 | 7:15386114 | 36273 | 7:15463697 | 36274 | 7:15501033 | 36283 | 7:15718347 | 36297 | 7:16146481 |
| 36298 | 7:16189628 | 36300 | 7:16237783 | 36305 | 7:16292747 | 36313 | 7:16521674 | 36314 | 7:16522621 |
| 36315 | 7:16524699 | 36317 | 7:16531552 | 36318 | 7:16534211 | 36319 | 7:16535641 | 36323 | 7:16561541 |
| 36359 | 7:17538355 | 36366 | 7:17643702 | 36367 | 7:17694868 | 36368 | 7:17698407 | 36369 | 7:17698410 |
| 36371 | 7:17712921 | 36389 | 7:18292860 | 36395 | 7:18386812 | 36397 | 7:18404440 | 36398 | 7:18434206 |
| 36399 | 7:18443286 | 36401 | 7:18446615 | 36402 | 7:18464757 | 36408 | 7:18658963 | 36411 | 7:18704573 |
| 36412 | 7:18706088 | 36413 | 7:18777233 | 36414 | 7:18826386 | 36417 | 7:18862062 | 36424 | 7:19108968 |
| 36425 | 7:19113510 | 36426 | 7:19115360 | 36428 | 7:19141191 | 36431 | 7:19189844 | 36436 | 7:19259287 |
| 36437 | 7:19261276 | 36438 | 7:19270709 | 36439 | 7:19272732 | 36443 | 7:19317767 | 36446 | 7:19337660 |
| 36449 | 7:19501368 | 36451 | 7:19560991 | 36452 | 7:19570534 | 36453 | 7:19601934 | 36454 | 7:19624357 |
| 36455 | 7:19629075 | 36456 | 7:19634135 | 36457 | 7:19674365 | 36459 | 7:19694033 | 36460 | 7:19709157 |
| 36462 | 7:19723388 | 36463 | 7:19742259 | 36464 | 7:19745894 | 36465 | 7:19749906 | 36468 | 7:19770884 |
| 36469 | 7:19782434 | 36470 | 7:19795534 | 36471 | 7:19811128 | 36473 | 7:19832477 | 36474 | 7:19846412 |
| 36475 | 7:19854110 | 36476 | 7:19861073 | 36477 | 7:19870302 | 36478 | 7:19874067 | 36479 | 7:19877997 |
| 36480 | 7:19882846 | 36481 | 7:19900064 | 36482 | 7:19904145 | 36483 | 7:19920619 | 36484 | 7:19925859 |
| 36485 | 7:19929090 | 36486 | 7:19934553 | 36487 | 7:19940802 | 36488 | 7:19948369 | 36489 | 7:19954948 |
| 36490 | 7:19962302 | 36491 | 7:19974241 | 36492 | 7:19977349 | 36494 | 7:20018689 | 36495 | 7:20036738 |
| 36496 | 7:20053188 | 36497 | 7:20057534 | 36498 | 7:20076160 | 36507 | 7:20236317 | 36512 | 7:20288732 |
| 36513 | 7:20318995 | 36514 | 7:20324208 | 36515 | 7:20324611 | 36517 | 7:20355414 | 36518 | 7:20356311 |
| 36519 | 7:20356363 | 36520 | 7:20356383 | 36521 | 7:20366443 | 36522 | 7:20366466 | 36531 | 7:20582777 |
| 36532 | 7:20591858 | 36534 | 7:20634478 | 36541 | 7:20805581 | 36543 | 7:20890886 | 36544 | 7:20890946 |
| 36547 | 7:20913247 | 36549 | 7:20940338 | 36558 | 7:21139922 | 36565 | 7:21283821 | 36570 | 7:21380240 |
| 36571 | 7:21386399 | 36573 | 7:21403549 | 36596 | 7:21986701 | 36601 | 7:22039959 | 36613 | 7:22387715 |
| 36624 | 7:22566377 | 36625 | 7:22566448 | 36627 | 7:22594692 | 36628 | 7:22594727 | 36633 | 7:22680039 |
| 36635 | 7:22786294 | 36637 | 7:22926260 | 36645 | 7:23219383 | 36648 | 7:23248102 | 36655 | 7:23451699 |
| 36702 | 7:24500311 | 36711 | 7:24783223 | 36714 | 7:24910871 | 36745 | 7:25616363 | 36749 | 7:25654878 |
| 36752 | 7:25731016 | 36753 | 7:25768089 | 36757 | 7:25800631 | 36758 | 7:25832593 | 36764 | 7:25936782 |
| 36766 | 7:26024639 | 36777 | 7:26292161 | 36778 | 7:26308990 | 36779 | 7:26319916 | 36782 | 7:26387075 |
| 36784 | 7:26402172 | 36788 | 7:26497485 | 36790 | 7:26557163 | 36791 | 7:26574444 | 36792 | 7:26577081 |
| 36793 | 7:26577087 | 36794 | 7:26578715 | 36796 | 7:26594068 | 36797 | 7:26594073 | 36798 | 7:26594090 |
| 36801 | 7:26638130 | 36805 | 7:26769987 | 36806 | 7:26769998 | 36807 | 7:26770016 | 36811 | 7:26823826 |
| 36814 | 7:26881081 | 36815 | 7:26898868 | 36821 | 7:27031629 | 36825 | 7:27060226 | 36832 | 7:27198824 |
| 36840 | 7:27423246 | 36841 | 7:27434826 | 36848 | 7:27596371 | 36855 | 7:27733288 | 36862 | 7:27870224 |
| 36865 | 7:28003332 | 36867 | 7:28049997 | 36868 | 7:28050030 | 36869 | 7:28050091 | 36870 | 7:28050098 |
| 36886 | 7:28544979 | 36893 | 7:28690143 | 36895 | 7:28700180 | 36897 | 7:28729276 | 36900 | 7:28830783 |
| 36902 | 7:28879641 | 36908 | 7:29036081 | 36912 | 7:29112331 | 36913 | 7:29178982 | 36922 | 7:29366546 |
| 36924 | 7:29452732 | 36934 | 7:29594179 | 36936 | 7:29632089 | 36938 | 7:29666350 | 36953 | 7:29976577 |
| 36956 | 7:30094509 | 36968 | 7:30358105 | 36971 | 7:30403065 | 36974 | 7:30553468 | 36977 | 7:30644004 |
| 36981 | 7:30714382 | 36982 | 7:30714446 | 36983 | 7:30756401 | 36984 | 7:30807818 | 36988 | 7:30863698 |
| 36992 | 7:30906898 | 36995 | 7:30991586 | 36998 | 7:31107264 | 37025 | 7:31626205 | 37029 | 7:31642354 |
| 37030 | 7:31642496 | 37031 | 7:31643684 | 37032 | 7:31643707 | 37037 | 7:31676529 | 37039 | 7:31699035 |
| 37065 | 7:32314617 | 37080 | 7:32688342 | 37081 | 7:32709556 | 37085 | 7:32796768 | 37096 | 7:33201093 |
| 37097 | 7:33201168 | 37119 | 7:33724498 | 37122 | 7:33761341 | 37123 | 7:33814744 | 37124 | 7:33814750 |
| 37125 | 7:33814773 | 37126 | 7:33814812 | 37129 | 7:33866953 | 37153 | 7:34486136 | 37154 | 7:34502493 |
| 37163 | 7:34781679 | 37170 | 7:34951198 | 37172 | 7:34967857 | 37173 | 7:34967898 | 37176 | 7:35028517 |
| 37182 | 7:35229836 | 37199 | 7:35718668 | 37219 | 7:36283231 | 37220 | 7:36308740 | 37231 | 7:36510620 |
| 37238 | 7:36626658 | 37239 | 7:36666622 | 37265 | 7:37451557 | 37266 | 7:37459216 | 37269 | 7:37490139 |
| 37273 | 7:37534315 | 37277 | 7:37590371 | 37281 | 7:37620741 | 37287 | 7:37840929 | 37291 | 7:37934995 |
| 37297 | 7:38048904 | 37298 | 7:38081521 | 37299 | 7:38087100 | 37300 | 7:38093801 | 37301 | 7:38101864 |
| 37302 | 7:38124565 | 37303 | 7:38128610 | 37304 | 7:38129977 | 37314 | 7:38325007 | 37316 | 7:38360533 |
| 37317 | 7:38365253 | 37318 | 7:38389751 | 37321 | 7:38404339 | 37322 | 7:38421353 | 37323 | 7:38429558 |
| 37327 | 7:38515000 | 37328 | 7:38534271 | 37333 | 7:38753839 | 37338 | 7:38815021 | 37343 | 7:38952074 |
| 37355 | 7:39351372 | 37356 | 7:39362501 | 37359 | 7:39455426 | 37368 | 7:39695685 | 37372 | 7:39721571 |
| 37373 | 7:39721626 | 37381 | 7:40016354 | 37385 | 7:40095095 | 37388 | 7:40114076 | 37389 | 7:40149307 |
| 37390 | 7:40151612 | 37396 | 7:40294613 | 37397 | 7:40294728 | 37398 | 7:40294729 | 37399 | 7:40294731 |
| 37400 | 7:40294744 | 37401 | 7:40294745 | 37404 | 7:40323542 | 37407 | 7:40355854 | 37408 | 7:40360599 |
| 37409 | 7:40361142 | 37410 | 7:40361184 | 37411 | 7:40361244 | 37412 | 7:40361336 | 37413 | 7:40372170 |
| 37414 | 7:40372221 | 37415 | 7:40385848 | 37416 | 7:40441629 | 37419 | 7:40507729 | 37420 | 7:40508207 |
| 37423 | 7:40603077 | 37424 | 7:40603099 | 37425 | 7:40604704 | 37426 | 7:40605637 | 37427 | 7:40606059 |
| 37429 | 7:40622112 | 37436 | 7:40917929 | 37438 | 7:40932803 | 37439 | 7:40933182 | 37440 | 7:40933299 |
| 37441 | 7:40945845 | 37442 | 7:40946121 | 37443 | 7:40946770 | 37444 | 7:40957931 | 37446 | 7:40958025 |
| 37447 | 7:40960932 | 37453 | 7:41290614 | 37454 | 7:41322935 | 37461 | 7:41552605 | 37462 | 7:41552746 |
| 37463 | 7:41553267 | 37464 | 7:41553442 | 37467 | 7:41694074 | 37468 | 7:41694108 | 37470 | 7:41791738 |
| 37472 | 7:41828300 | 37473 | 7:41828310 | 37474 | 7:41828476 | 37475 | 7:41828489 | 37476 | 7:41828529 |
| 37477 | 7:41828531 | 37478 | 7:41828618 | 37483 | 7:42132719 | 37495 | 7:42627355 | 37496 | 7:42628923 |
| 37498 | 7:42659874 | 37499 | 7:42659928 | 37509 | 7:43070478 | 37510 | 7:43070495 | 37511 | 7:43073489 |
| 37512 | 7:43074460 | 37513 | 7:43074485 | 37514 | 7:43075276 | 37515 | 7:43075327 | 37516 | 7:43075329 |
| 37517 | 7:43075823 | 37518 | 7:43075911 | 37519 | 7:43077087 | 37522 | 7:43114270 | 37525 | 7:43247821 |
| 37526 | 7:43258641 | 37531 | 7:43495660 | 37532 | 7:43504923 | 37533 | 7:43504924 | 37534 | 7:43509552 |
| 37535 | 7:43518518 | 37536 | 7:43526310 | 37537 | 7:43541646 | 37539 | 7:43547038 | 37540 | 7:43551313 |
| 37541 | 7:43573739 | 37542 | 7:43602807 | 37543 | 7:43616877 | 37546 | 7:43695498 | 37548 | 7:43809877 |
| 37549 | 7:43811263 | 37550 | 7:43812894 | 37552 | 7:43875604 | 37558 | 7:44162642 | 37563 | 7:44416303 |
| 37566 | 7:44610929 | 37568 | 7:44639553 | 37569 | 7:44666191 | 37572 | 7:44764045 | 37573 | 7:44764061 |
| 37574 | 7:44789031 | 37575 | 7:44789067 | 37576 | 7:44789088 | 37584 | 7:44943643 | 37585 | 7:45003392 |
| 37591 | 7:45101749 | 37599 | 7:45350646 | 37600 | 7:45355058 | 37605 | 7:45445789 | 37614 | 7:45661047 |
| 37619 | 7:45951627 | 37620 | 7:45994439 | 37621 | 7:45997784 | 37623 | 7:46123501 | 37624 | 7:46133078 |
| 37637 | 7:46424169 | 37649 | 7:46722010 | 37655 | 7:46847399 | 37657 | 7:46881748 | 37659 | 7:46963800 |
| 37660 | 7:46965141 | 37661 | 7:46965986 | 37662 | 7:46966917 | 37663 | 7:46969143 | 37678 | 7:47071431 |
| 37680 | 7:47200029 | 37694 | 7:47663425 | 37696 | 7:47675422 | 37697 | 7:47681261 | 37701 | 7:47796975 |
| 37722 | 7:48566329 | 37730 | 7:48673881 | 37771 | 7:50021166 | 37780 | 7:50639584 | 37792 | 7:51149038 |
| 37796 | 7:51377867 | 37798 | 7:51400935 | 37801 | 7:51429431 | 37803 | 7:51450812 | 37811 | 7:51923554 |
| 37812 | 7:52003650 | 37815 | 7:52173201 | 37817 | 7:52176528 | 37818 | 7:52177126 | 37819 | 7:52179498 |
| 37820 | 7:52181770 | 37821 | 7:52182473 | 37822 | 7:52183049 | 37823 | 7:52183519 | 37824 | 7:52186624 |
| 37825 | 7:52193152 | 37826 | 7:52196823 | 37827 | 7:52197432 | 37828 | 7:52198757 | 37829 | 7:52199982 |
| 37830 | 7:52207035 | 37831 | 7:52210371 | 37833 | 7:52224828 | 37834 | 7:52225521 | 37835 | 7:52227549 |
| 37836 | 7:52227592 | 37838 | 7:52249785 | 37839 | 7:52251276 | 37840 | 7:52251297 | 37841 | 7:52251426 |
| 37842 | 7:52251546 | 37843 | 7:52251618 | 37844 | 7:52251861 | 37845 | 7:52252987 | 37846 | 7:52263699 |
| 37847 | 7:52264079 | 37848 | 7:52264430 | 37849 | 7:52264511 | 37850 | 7:52264761 | 37851 | 7:52266394 |
| 37852 | 7:52282234 | 37853 | 7:52289509 | 37854 | 7:52291347 | 37855 | 7:52293091 | 37856 | 7:52294120 |
| 37857 | 7:52295099 | 37858 | 7:52296408 | 37859 | 7:52296468 | 37860 | 7:52298384 | 37861 | 7:52299162 |
| 37862 | 7:52300813 | 37863 | 7:52303764 | 37864 | 7:52305125 | 37865 | 7:52305218 | 37866 | 7:52308210 |
| 37867 | 7:52308534 | 37868 | 7:52308687 | 37869 | 7:52309821 | 37870 | 7:52339995 | 37871 | 7:52344653 |
| 37872 | 7:52345469 | 37873 | 7:52348265 | 37874 | 7:52349069 | 37878 | 7:52567577 | 37879 | 7:52610395 |
| 37880 | 7:52611688 | 37884 | 7:52667909 | 37895 | 7:52931391 | 37898 | 7:53021130 | 37904 | 7:53089284 |
| 37908 | 7:53195304 | 37920 | 7:53551894 | 37923 | 7:53581842 | 37924 | 7:53587134 | 37928 | 7:53866382 |
| 37931 | 7:53935965 | 37934 | 7:53979466 | 37938 | 7:54160608 | 37943 | 7:54227894 | 37944 | 7:54238880 |
| 37949 | 7:54401837 | 37950 | 7:54414044 | 37952 | 7:54475662 | 37983 | 7:55275367 | 37984 | 7:55310804 |
| 37992 | 7:55508633 | 37997 | 7:55613829 | 38014 | 7:56033788 | 38027 | 7:56270767 | 38054 | 7:57224558 |
| 38056 | 7:57261914 | 38060 | 7:57381076 | 38067 | 7:57701637 | 38068 | 7:57702653 | 38079 | 7:57942576 |
| 38099 | 7:58504274 | 38100 | 7:58564407 | 38103 | 7:58594636 | 38106 | 7:58736129 | 38112 | 7:58873109 |
| 38114 | 7:58937938 | 38118 | 7:59041978 | 38119 | 7:59042119 | 38120 | 7:59044072 | 38121 | 7:59055433 |
| 38123 | 7:59057188 | 38124 | 7:59057293 | 38127 | 7:59101278 | 38128 | 7:59111622 | 38132 | 7:59143825 |
| 38141 | 7:59349638 | 38142 | 7:59349648 | 38143 | 7:59357865 | 38144 | 7:59366049 | 38147 | 7:59372999 |
| 38148 | 7:59373109 | 38149 | 7:59373145 | 38150 | 7:59373890 | 38151 | 7:59373911 | 38152 | 7:59373987 |
| 38154 | 7:59405369 | 38161 | 7:59476011 | 38173 | 7:59813378 | 38174 | 7:59851233 | 38176 | 7:59880943 |
| 38179 | 7:59979404 | 38180 | 7:59993171 | 38183 | 7:60051047 | 38187 | 7:60113080 | 38190 | 7:60121204 |
| 38191 | 7:60146715 | 38192 | 7:60159491 | 38202 | 7:60502888 | 38205 | 7:60568272 | 38213 | 7:60834283 |
| 38220 | 7:60976830 | 38222 | 7:60986666 | 38223 | 7:60989492 | 38225 | 7:61027791 | 38226 | 7:61067590 |
| 38227 | 7:61075693 | 38230 | 7:61121888 | 38231 | 7:61125177 | 38234 | 7:61147314 | 38235 | 7:61157706 |
| 38236 | 7:61175816 | 38239 | 7:61222597 | 38241 | 7:61229844 | 38242 | 7:61229883 | 38243 | 7:61234323 |
| 38244 | 7:61234418 | 38245 | 7:61240972 | 38250 | 7:61334946 | 38251 | 7:61352203 | 38252 | 7:61373465 |
| 38255 | 7:61436635 | 38262 | 7:61491892 | 38265 | 7:61518179 | 38267 | 7:61555353 | 38271 | 7:61582159 |
| 38275 | 7:61778550 | 38282 | 7:62019992 | 38284 | 7:62026620 | 38292 | 7:62285057 | 38294 | 7:62296401 |
| 38298 | 7:62370721 | 38300 | 7:62436131 | 38302 | 7:62469301 | 38303 | 7:62475081 | 38304 | 7:62508971 |
| 38307 | 7:62555449 | 38310 | 7:62601203 | 38311 | 7:62614056 | 38315 | 7:62677859 | 38329 | 7:62720115 |
| 38340 | 7:62942190 | 38354 | 7:63240676 | 38365 | 7:63685100 | 38378 | 7:63974136 | 38389 | 7:64332584 |
| 38410 | 7:64916386 | 38416 | 7:65002376 | 38419 | 7:65066376 | 38421 | 7:65074340 | 38433 | 7:65274641 |
| 38435 | 7:65330563 | 38439 | 7:65403716 | 38444 | 7:65453830 | 38453 | 7:65721041 | 38464 | 7:65972504 |
| 38466 | 7:65984655 | 38467 | 7:66008306 | 38468 | 7:66011532 | 38471 | 7:66093673 | 38472 | 7:66101631 |
| 38473 | 7:66102398 | 38474 | 7:66104176 | 38475 | 7:66105023 | 38476 | 7:66105843 | 38477 | 7:66108875 |
| 38478 | 7:66109426 | 38479 | 7:66110363 | 38480 | 7:66112663 | 38481 | 7:66113133 | 38482 | 7:66114775 |
| 38483 | 7:66117158 | 38484 | 7:66117914 | 38485 | 7:66118467 | 38486 | 7:66120071 | 38487 | 7:66121243 |
| 38488 | 7:66122354 | 38489 | 7:66122876 | 38490 | 7:66124410 | 38491 | 7:66125761 | 38492 | 7:66126649 |
| 38493 | 7:66130675 | 38494 | 7:66134907 | 38495 | 7:66146626 | 38496 | 7:66158927 | 38512 | 7:66629354 |
| 38513 | 7:66638097 | 38514 | 7:66640996 | 38519 | 7:66842880 | 38523 | 7:66907257 | 38532 | 7:67112605 |
| 38540 | 7:67368109 | 38549 | 7:67650949 | 38550 | 7:67663732 | 38551 | 7:67677225 | 38556 | 7:67803883 |
| 38582 | 7:68478076 | 38595 | 7:68892260 | 38597 | 7:68928960 | 38598 | 7:68949630 | 38601 | 7:68987039 |
| 38602 | 7:68998753 | 38604 | 7:69001013 | 38606 | 7:69049520 | 38611 | 7:69144296 | 38615 | 7:69172516 |
| 38618 | 7:69198573 | 38624 | 7:69399622 | 38627 | 7:69502402 | 38628 | 7:69503500 | 38631 | 7:69517359 |
| 38645 | 7:69896069 | 38649 | 7:69988788 | 38652 | 7:70030654 | 38654 | 7:70060807 | 38660 | 7:70128127 |
| 38675 | 7:70701637 | 38676 | 7:70732259 | 38692 | 7:71222059 | 38695 | 7:71353347 | 38704 | 7:71545001 |
| 38705 | 7:71558907 | 38733 | 7:72053452 | 38737 | 7:72207894 | 38738 | 7:72232062 | 38744 | 7:72362246 |
| 38759 | 7:72898450 | 38762 | 7:72962658 | 38770 | 7:73184647 | 38775 | 7:73473535 | 38793 | 7:73942821 |
| 38805 | 7:74306314 | 38806 | 7:74306327 | 38807 | 7:74306379 | 38809 | 7:74321645 | 38818 | 7:74667948 |
| 38824 | 7:74772424 | 38825 | 7:74834470 | 38827 | 7:74855482 | 38828 | 7:74855556 | 38829 | 7:74861008 |
| 38834 | 7:74922579 | 38835 | 7:74934509 | 38836 | 7:74967965 | 38838 | 7:75008858 | 38839 | 7:75009945 |
| 38844 | 7:75141363 | 38845 | 7:75145201 | 38846 | 7:75157547 | 38848 | 7:75201764 | 38860 | 7:75609551 |
| 38862 | 7:75636089 | 38863 | 7:75677642 | 38864 | 7:75677689 | 38871 | 7:75802119 | 38872 | 7:75814579 |
| 38877 | 7:75942645 | 38886 | 7:76249772 | 38887 | 7:76260400 | 38888 | 7:76279595 | 38890 | 7:76306268 |
| 38891 | 7:76342697 | 38892 | 7:76347497 | 38894 | 7:76370749 | 38898 | 7:76449574 | 38910 | 7:76725621 |
| 38911 | 7:76765051 | 38914 | 7:76875833 | 38915 | 7:76875983 | 38919 | 7:77004376 | 38939 | 7:77408753 |
| 38960 | 7:77903085 | 38966 | 7:77985310 | 38970 | 7:78110195 | 38974 | 7:78196572 | 38975 | 7:78196650 |
| 38977 | 7:78217247 | 39034 | 7:79217925 | 39044 | 7:79409160 | 39050 | 7:79575037 | 39051 | 7:79575100 |
| 39055 | 7:79623429 | 39057 | 7:79676699 | 39061 | 7:79735833 | 39062 | 7:79735898 | 39070 | 7:79930582 |
| 39071 | 7:79953397 | 39074 | 7:80074984 | 39081 | 7:80181414 | 39086 | 7:80322587 | 39091 | 7:80397326 |
| 39092 | 7:80423038 | 39094 | 7:80460624 | 39095 | 7:80461082 | 39097 | 7:80469480 | 39098 | 7:80471094 |
| 39100 | 7:80478643 | 39101 | 7:80503605 | 39102 | 7:80503765 | 39104 | 7:80509969 | 39105 | 7:80514758 |
| 39109 | 7:80546745 | 39113 | 7:80594054 | 39116 | 7:80662788 | 39130 | 7:81016584 | 39138 | 7:81135163 |
| 39139 | 7:81142008 | 39140 | 7:81142055 | 39141 | 7:81142172 | 39142 | 7:81174306 | 39144 | 7:81184476 |
| 39145 | 7:81184486 | 39151 | 7:81313732 | 39158 | 7:81392597 | 39159 | 7:81392623 | 39160 | 7:81392634 |
| 39161 | 7:81392650 | 39162 | 7:81392653 | 39163 | 7:81392665 | 39170 | 7:81654928 | 39175 | 7:81798512 |
| 39186 | 7:82216490 | 39187 | 7:82219959 | 39189 | 7:82230892 | 39190 | 7:82248295 | 39198 | 7:82549435 |
| 39200 | 7:82554305 | 39206 | 7:82662900 | 39207 | 7:82662928 | 39208 | 7:82662929 | 39214 | 7:82764185 |
| 39224 | 7:83044183 | 39244 | 7:83544032 | 39264 | 7:84108168 | 39265 | 7:84108203 | 39266 | 7:84108212 |
| 39267 | 7:84118928 | 39269 | 7:84144485 | 39273 | 7:84207697 | 39275 | 7:84239228 | 39279 | 7:84354399 |
| 39280 | 7:84354448 | 39284 | 7:84424155 | 39285 | 7:84450073 | 39288 | 7:84509794 | 39301 | 7:85123948 |
| 39307 | 7:85293142 | 39308 | 7:85293161 | 39309 | 7:85293169 | 39323 | 7:85845513 | 39324 | 7:85845564 |
| 39325 | 7:85875827 | 39328 | 7:85965693 | 39337 | 7:86289890 | 39338 | 7:86289929 | 39340 | 7:86348377 |
| 39341 | 7:86348400 | 39345 | 7:86462459 | 39348 | 7:86544147 | 39349 | 7:86566118 | 39353 | 7:86638215 |
| 39379 | 7:87401188 | 39388 | 7:87608805 | 39389 | 7:87608884 | 39390 | 7:87608885 | 39391 | 7:87608911 |
| 39394 | 7:87779029 | 39395 | 7:87841745 | 39403 | 7:88117633 | 39411 | 7:88379769 | 39419 | 7:88587478 |
| 39427 | 7:88790932 | 39428 | 7:88796649 | 39433 | 7:88942654 | 39435 | 7:88966573 | 39436 | 7:89035670 |
| 39440 | 7:89116248 | 39445 | 7:89148646 | 39446 | 7:89184830 | 39450 | 7:89247931 | 39454 | 7:89369998 |
| 39478 | 7:90049487 | 39483 | 7:90127121 | 39486 | 7:90166602 | 39487 | 7:90166627 | 39488 | 7:90167634 |
| 39496 | 7:90329807 | 39511 | 7:90598533 | 39514 | 7:90688919 | 39523 | 7:90779825 | 39558 | 7:90831595 |
| 39565 | 7:90845831 | 39596 | 7:91489178 | 39611 | 7:91726773 | 39653 | 7:92762601 | 39671 | 7:93433611 |
| 39679 | 7:93718483 | 39680 | 7:93727310 | 39682 | 7:93774135 | 39693 | 7:94215835 | 39694 | 7:94269949 |
| 39699 | 7:94382446 | 39703 | 7:94439368 | 39708 | 7:94574747 | 39717 | 7:94849950 | 39718 | 7:94854494 |
| 39719 | 7:94854604 | 39720 | 7:94854627 | 39724 | 7:94945483 | 39729 | 7:95020011 | 39730 | 7:95021828 |
| 39733 | 7:95079030 | 39735 | 7:95116563 | 39736 | 7:95158884 | 39743 | 7:95405216 | 39754 | 7:95645495 |
| 39767 | 7:95958530 | 39771 | 7:96074523 | 39772 | 7:96087310 | 39775 | 7:96098877 | 39778 | 7:96109770 |
| 39781 | 7:96127017 | 39782 | 7:96127023 | 39783 | 7:96127094 | 39784 | 7:96140233 | 39785 | 7:96140250 |
| 39786 | 7:96140274 | 39787 | 7:96140286 | 39788 | 7:96140316 | 39789 | 7:96140325 | 39790 | 7:96146797 |
| 39791 | 7:96152289 | 39793 | 7:96169102 | 39806 | 7:96180272 | 39807 | 7:96180277 | 39808 | 7:96180291 |
| 39810 | 7:96196608 | 39811 | 7:96196642 | 39813 | 7:96202522 | 39829 | 7:96220995 | 39831 | 7:96221193 |
| 39868 | 7:96278344 | 39911 | 7:96314599 | 39912 | 7:96315078 | 39934 | 7:96355415 | 39939 | 7:96370734 |
| 39941 | 7:96387738 | 39950 | 7:96555097 | 39952 | 7:96601732 | 39954 | 7:96613640 | 39985 | 7:97508892 |
| 39986 | 7:97508905 | 39988 | 7:97541930 | 39990 | 7:97603739 | 39991 | 7:97629287 | 39997 | 7:97747540 |
| 40003 | 7:97959533 | 40019 | 7:98300666 | 40020 | 7:98348623 | 40021 | 7:98348636 | 40028 | 7:98543164 |
| 40048 | 7:99313338 | 40070 | 7:99845218 | 40072 | 7:99865657 | 40075 | 7:99914902 | 40082 | 7:100079502 |
| 40083 | 7:100082753 | 40086 | 7:100118655 | 40087 | 7:100118835 | 40088 | 7:100136909 | 40089 | 7:100144865 |
| 40098 | 7:100358134 | 40099 | 7:100395807 | 40116 | 7:100797612 | 40129 | 7:101254706 | 40133 | 7:101358810 |
| 40139 | 7:101563788 | 40142 | 7:101606446 | 40143 | 7:101606448 | 40147 | 7:101667972 | 40153 | 7:101808436 |
| 40154 | 7:101857168 | 40157 | 7:102004868 | 40164 | 7:102285994 | 40173 | 7:102580748 | 40174 | 7:102582243 |
| 40175 | 7:102585831 | 40176 | 7:102589674 | 40177 | 7:102594188 | 40178 | 7:102599028 | 40179 | 7:102609048 |
| 40181 | 7:102620944 | 40192 | 7:102888318 | 40196 | 7:102988342 | 40198 | 7:103035483 | 40200 | 7:103047606 |
| 40219 | 7:103414818 | 40280 | 7:105164324 | 40292 | 7:105387524 | 40298 | 7:105535552 | 40322 | 7:106096645 |
| 40326 | 7:106249472 | 40327 | 7:106254713 | 40348 | 7:106643847 | 40351 | 7:106742504 | 40352 | 7:106742506 |
| 40360 | 7:106848432 | 40362 | 7:106869396 | 40369 | 7:107016860 | 40370 | 7:107023813 | 40373 | 7:107095006 |
| 40376 | 7:107124835 | 40383 | 7:107308750 | 40403 | 7:108184476 | 40407 | 7:108325718 | 40408 | 7:108337537 |
| 40418 | 7:108563367 | 40431 | 7:108821941 | 40433 | 7:108869029 | 40439 | 7:108994618 | 40441 | 7:109029038 |
| 40442 | 7:109029077 | 40443 | 7:109029102 | 40458 | 7:109275280 | 40480 | 7:109999314 | 40498 | 8:373283 |
| 40506 | 8:643950 | 40510 | 8:764642 | 40511 | 8:780102 | 40514 | 8:800236 | 40515 | 8:816422 |
| 40517 | 8:844855 | 40520 | 8:882426 | 40521 | 8:883861 | 40535 | 8:1082935 | 40536 | 8:1086523 |
| 40548 | 8:1362737 | 40552 | 8:1579017 | 40564 | 8:1757679 | 40571 | 8:1832243 | 40579 | 8:1939311 |
| 40594 | 8:2317585 | 40606 | 8:2582570 | 40616 | 8:3008685 | 40620 | 8:3178994 | 40621 | 8:3219275 |
| 40630 | 8:3400647 | 40631 | 8:3416328 | 40632 | 8:3416882 | 40667 | 8:4251182 | 40668 | 8:4273155 |
| 40669 | 8:4274587 | 40670 | 8:4276578 | 40671 | 8:4280150 | 40672 | 8:4283775 | 40685 | 8:4639116 |
| 40687 | 8:4644380 | 40688 | 8:4644486 | 40690 | 8:4708141 | 40693 | 8:4759903 | 40698 | 8:4908669 |
| 40707 | 8:5249662 | 40708 | 8:5270683 | 40709 | 8:5270703 | 40710 | 8:5270802 | 40711 | 8:5270815 |
| 40712 | 8:5332706 | 40728 | 8:5760796 | 40735 | 8:5869022 | 40741 | 8:6027760 | 40765 | 8:6756792 |
| 40766 | 8:6764623 | 40785 | 8:7181322 | 40786 | 8:7181335 | 40803 | 8:7581528 | 40805 | 8:7596757 |
| 40806 | 8:7598915 | 40807 | 8:7601383 | 40808 | 8:7603103 | 40809 | 8:7611610 | 40810 | 8:7618608 |
| 40811 | 8:7619933 | 40812 | 8:7620831 | 40813 | 8:7625351 | 40818 | 8:7818361 | 40826 | 8:8001917 |
| 40832 | 8:8175029 | 40842 | 8:8393306 | 40844 | 8:8477216 | 40848 | 8:8618577 | 40857 | 8:8769353 |
| 40866 | 8:9042180 | 40868 | 8:9063209 | 40869 | 8:9119208 | 40889 | 8:9619039 | 40890 | 8:9619107 |
| 40891 | 8:9619135 | 40892 | 8:9623038 | 40893 | 8:9627034 | 40894 | 8:9627109 | 40895 | 8:9627275 |
| 40896 | 8:9627321 | 40905 | 8:9890341 | 40906 | 8:9901901 | 40910 | 8:10031350 | 40917 | 8:10339279 |
| 40925 | 8:10604669 | 40931 | 8:10696791 | 40934 | 8:10712286 | 40935 | 8:10712485 | 40936 | 8:10712536 |
| 40939 | 8:10783394 | 40942 | 8:10854210 | 40943 | 8:10854709 | 40952 | 8:11049154 | 40955 | 8:11128689 |
| 40980 | 8:11615963 | 40981 | 8:11626767 | 40983 | 8:11655158 | 40985 | 8:11693820 | 40990 | 8:11853891 |
| 41001 | 8:12089230 | 41008 | 8:12344836 | 41012 | 8:12433144 | 41026 | 8:12914075 | 41036 | 8:13348241 |
| 41038 | 8:13393062 | 41047 | 8:13722063 | 41097 | 8:15148225 | 41103 | 8:15333923 | 41109 | 8:15473403 |
| 41113 | 8:15497145 | 41114 | 8:15497203 | 41115 | 8:15497248 | 41116 | 8:15533442 | 41120 | 8:15590887 |
| 41121 | 8:15590953 | 41124 | 8:15685122 | 41128 | 8:15765706 | 41133 | 8:15967637 | 41143 | 8:16338822 |
| 41145 | 8:16399613 | 41155 | 8:16592732 | 41158 | 8:16699848 | 41162 | 8:16791579 | 41163 | 8:16792851 |
| 41171 | 8:16926726 | 41174 | 8:16945156 | 41178 | 8:17032464 | 41179 | 8:17032475 | 41180 | 8:17032488 |
| 41182 | 8:17048252 | 41183 | 8:17055738 | 41184 | 8:17055749 | 41185 | 8:17055775 | 41186 | 8:17056869 |
| 41187 | 8:17061563 | 41188 | 8:17064508 | 41193 | 8:17304347 | 41195 | 8:17305220 | 41196 | 8:17307048 |
| 41197 | 8:17307131 | 41206 | 8:17548023 | 41231 | 8:18280322 | 41242 | 8:18613707 | 41246 | 8:18768280 |
| 41257 | 8:19052433 | 41288 | 8:19935582 | 41301 | 8:20342622 | 41342 | 8:21329932 | 41361 | 8:21984030 |
| 41363 | 8:22082837 | 41364 | 8:22100760 | 41365 | 8:22113575 | 41396 | 8:23324599 | 41401 | 8:23427337 |
| 41421 | 8:24057660 | 41434 | 8:24296988 | 41440 | 8:24394806 | 41442 | 8:24405999 | 41444 | 8:24443570 |
| 41450 | 8:24643364 | 41452 | 8:24674588 | 41454 | 8:24687934 | 41455 | 8:24689851 | 41456 | 8:24692069 |
| 41457 | 8:24700212 | 41459 | 8:24720840 | 41462 | 8:24752172 | 41466 | 8:24771365 | 41467 | 8:24786333 |
| 41468 | 8:24793655 | 41469 | 8:24802374 | 41470 | 8:24812615 | 41471 | 8:24820473 | 41472 | 8:24827262 |
| 41475 | 8:24874038 | 41476 | 8:24875382 | 41477 | 8:24877159 | 41478 | 8:24877700 | 41479 | 8:24878235 |
| 41480 | 8:24878922 | 41481 | 8:24888678 | 41482 | 8:24891103 | 41483 | 8:24895594 | 41485 | 8:24933323 |
| 41486 | 8:24938771 | 41487 | 8:24940757 | 41488 | 8:24955657 | 41489 | 8:24957890 | 41490 | 8:24961237 |
| 41491 | 8:24966416 | 41492 | 8:24974478 | 41493 | 8:24979200 | 41494 | 8:24988001 | 41495 | 8:24992186 |
| 41496 | 8:24996358 | 41497 | 8:25006948 | 41498 | 8:25013387 | 41499 | 8:25035664 | 41500 | 8:25041072 |
| 41501 | 8:25059424 | 41506 | 8:25124953 | 41507 | 8:25126729 | 41509 | 8:25140406 | 41510 | 8:25144505 |
| 41511 | 8:25146183 | 41512 | 8:25148717 | 41513 | 8:25150222 | 41514 | 8:25152059 | 41515 | 8:25154593 |
| 41516 | 8:25157388 | 41517 | 8:25160394 | 41518 | 8:25165311 | 41519 | 8:25166943 | 41520 | 8:25169477 |
| 41522 | 8:25174754 | 41523 | 8:25177709 | 41524 | 8:25180230 | 41525 | 8:25185692 | 41526 | 8:25188439 |
| 41527 | 8:25198273 | 41528 | 8:25200755 | 41530 | 8:25214312 | 41531 | 8:25227176 | 41532 | 8:25233627 |
| 41533 | 8:25235124 | 41534 | 8:25238388 | 41535 | 8:25240252 | 41537 | 8:25243485 | 41538 | 8:25254140 |
| 41539 | 8:25257480 | 41540 | 8:25259038 | 41541 | 8:25261263 | 41542 | 8:25263852 | 41543 | 8:25265485 |
| 41544 | 8:25266937 | 41545 | 8:25268844 | 41546 | 8:25274693 | 41547 | 8:25282832 | 41548 | 8:25294627 |
| 41557 | 8:25487377 | 41558 | 8:25500162 | 41561 | 8:25551702 | 41580 | 8:25993221 | 41584 | 8:26067879 |
| 41602 | 8:26560396 | 41604 | 8:26591338 | 41614 | 8:26825483 | 41621 | 8:26909158 | 41631 | 8:27313786 |
| 41633 | 8:27383296 | 41637 | 8:27558131 | 41649 | 8:27894769 | 41652 | 8:27969801 | 41654 | 8:28067472 |
| 41664 | 8:28411601 | 41665 | 8:28411658 | 41670 | 8:28576597 | 41673 | 8:28652470 | 41678 | 8:28903569 |
| 41679 | 8:28935823 | 41691 | 8:29203761 | 41702 | 8:29452933 | 41706 | 8:29509148 | 41707 | 8:29543012 |
| 41728 | 8:29972472 | 41730 | 8:30031385 | 41734 | 8:30076221 | 41737 | 8:30253112 | 41750 | 8:30514176 |
| 41754 | 8:30666192 | 41757 | 8:30707536 | 41764 | 8:30964101 | 41765 | 8:30965820 | 41767 | 8:31027893 |
| 41769 | 8:31049984 | 41770 | 8:31056192 | 41773 | 8:31128535 | 41776 | 8:31166613 | 41780 | 8:31290392 |
| 41781 | 8:31300884 | 41785 | 8:31442029 | 41786 | 8:31453763 | 41790 | 8:31579785 | 41793 | 8:31643340 |
| 41801 | 8:31867242 | 41802 | 8:31895156 | 41859 | 8:33917004 | 41888 | 8:34615024 | 41894 | 8:34800931 |
| 41895 | 8:34800977 | 41896 | 8:34801009 | 41919 | 8:35445047 | 41923 | 8:35459817 | 41936 | 8:35833781 |
| 41938 | 8:35859948 | 41943 | 8:35930089 | 41953 | 8:36065706 | 41956 | 8:36096463 | 41958 | 8:36187712 |
| 41962 | 8:36233237 | 41965 | 8:36292654 | 41968 | 8:36343251 | 41985 | 8:36842382 | 41986 | 8:36851180 |
| 41995 | 8:37031806 | 41996 | 8:37054784 | 41998 | 8:37095387 | 42008 | 8:37407050 | 42010 | 8:37435996 |
| 42023 | 8:37797692 | 42024 | 8:37854305 | 42028 | 8:37928176 | 42044 | 8:38675399 | 42047 | 8:38691615 |
| 42056 | 8:39019428 | 42061 | 8:39290623 | 42067 | 8:39583278 | 42068 | 8:39584890 | 42069 | 8:39621719 |
| 42074 | 8:39696110 | 42075 | 8:39718793 | 42077 | 8:39768968 | 42078 | 8:39769005 | 42079 | 8:39809755 |
| 42081 | 8:39864901 | 42091 | 8:40003850 | 42101 | 8:40171823 | 42102 | 8:40219628 | 42103 | 8:40219668 |
| 42105 | 8:40257959 | 42106 | 8:40287413 | 42107 | 8:40306339 | 42109 | 8:40334191 | 42110 | 8:40334321 |
| 42114 | 8:40410096 | 42131 | 8:40743770 | 42135 | 8:40830870 | 42150 | 8:41247220 | 42152 | 8:41309293 |
| 42165 | 8:41611046 | 42168 | 8:41682500 | 42169 | 8:41693466 | 42170 | 8:41697104 | 42174 | 8:41759986 |
| 42176 | 8:41790111 | 42179 | 8:41858426 | 42180 | 8:41890619 | 42181 | 8:41897019 | 42199 | 8:42436416 |
| 42211 | 8:42709975 | 42212 | 8:42720374 | 42216 | 8:42880518 | 42220 | 8:42958668 | 42221 | 8:42958711 |
| 42222 | 8:42958836 | 42226 | 8:43016661 | 42236 | 8:43367984 | 42245 | 8:43812387 | 42246 | 8:43815004 |
| 42250 | 8:43957564 | 42255 | 8:43998136 | 42256 | 8:43998213 | 42258 | 8:44010856 | 42269 | 8:44431429 |
| 42273 | 8:44588721 | 42282 | 8:44879292 | 42283 | 8:44895474 | 42285 | 8:44916012 | 42295 | 8:45238924 |
| 42301 | 8:45399787 | 42304 | 8:45427010 | 42306 | 8:45447403 | 42309 | 8:45519975 | 42310 | 8:45544435 |
| 42314 | 8:45672590 | 42316 | 8:45691728 | 42321 | 8:45748288 | 42327 | 8:45881905 | 42328 | 8:45949542 |
| 42333 | 8:46053215 | 42334 | 8:46058853 | 42347 | 8:46184772 | 42352 | 8:46322367 | 42358 | 8:46514754 |
| 42362 | 8:46662471 | 42363 | 8:46688442 | 42367 | 8:46794717 | 42378 | 8:47069272 | 42379 | 8:47096555 |
| 42383 | 8:47242828 | 42384 | 8:47301441 | 42386 | 8:47301545 | 42396 | 8:47442795 | 42397 | 8:47454300 |
| 42398 | 8:47468132 | 42413 | 8:47827950 | 42416 | 8:47959914 | 42443 | 8:48870474 | 42452 | 8:48980291 |
| 42455 | 8:49060228 | 42456 | 8:49060267 | 42457 | 8:49060352 | 42458 | 8:49071203 | 42459 | 8:49082742 |
| 42465 | 8:49227434 | 42466 | 8:49248066 | 42470 | 8:49305124 | 42471 | 8:49328971 | 42472 | 8:49332342 |
| 42484 | 8:49820121 | 42490 | 8:50120835 | 42493 | 8:50158365 | 42496 | 8:50210739 | 42502 | 8:50343730 |
| 42505 | 8:50410404 | 42526 | 8:51083850 | 42529 | 8:51149379 | 42530 | 8:51154605 | 42537 | 8:51409152 |
| 42549 | 8:51645019 | 42553 | 8:51697635 | 42563 | 8:51983681 | 42569 | 8:52152405 | 42570 | 8:52157895 |
| 42573 | 8:52201603 | 42584 | 8:52496347 | 42593 | 8:52683991 | 42597 | 8:52775082 | 42606 | 8:53099576 |
| 42607 | 8:53099649 | 42608 | 8:53099684 | 42618 | 8:53465873 | 42622 | 8:53559314 | 42626 | 8:53663149 |
| 42628 | 8:53760217 | 42643 | 8:54223148 | 42650 | 8:54393899 | 42663 | 8:54661982 | 42665 | 8:54767805 |
| 42666 | 8:54767855 | 42676 | 8:54981068 | 42677 | 8:54981074 | 42678 | 8:54981156 | 42693 | 8:55456387 |
| 42697 | 8:55552471 | 42699 | 8:55552575 | 42707 | 8:55861711 | 42708 | 8:55861771 | 42709 | 8:55869592 |
| 42711 | 8:55889240 | 42718 | 8:56082094 | 42719 | 8:56082109 | 42723 | 8:56355997 | 42732 | 8:56476308 |
| 42736 | 8:56567532 | 42749 | 8:56898409 | 42752 | 8:56940553 | 42755 | 8:57067743 | 42756 | 8:57083802 |
| 42757 | 8:57097646 | 42769 | 8:57431848 | 42770 | 8:57436089 | 42771 | 8:57441513 | 42774 | 8:57491864 |
| 42777 | 8:57503925 | 42781 | 8:57561608 | 42784 | 8:57609698 | 42785 | 8:57624471 | 42794 | 8:58010694 |
| 42796 | 8:58065599 | 42799 | 8:58159003 | 42803 | 8:58281685 | 42817 | 8:58837870 | 42827 | 8:59185961 |
| 42831 | 8:59304332 | 42833 | 8:59343826 | 42843 | 8:59578341 | 42845 | 8:59585372 | 42846 | 8:59591203 |
| 42847 | 8:59595742 | 42853 | 8:59747078 | 42856 | 8:59839831 | 42861 | 8:59908902 | 42867 | 8:59979908 |
| 42869 | 8:59997834 | 42873 | 8:60218203 | 42874 | 8:60232849 | 42875 | 8:60252660 | 42877 | 8:60376601 |
| 42878 | 8:60387912 | 42880 | 8:60393687 | 42881 | 8:60407147 | 42882 | 8:60408080 | 42883 | 8:60413868 |
| 42885 | 8:60422237 | 42887 | 8:60434309 | 42888 | 8:60445473 | 42893 | 8:60483449 | 42901 | 8:60773154 |
| 42906 | 8:60847978 | 42928 | 8:61569513 | 42931 | 8:61676872 | 42953 | 8:62298007 | 42955 | 8:62361352 |
| 42956 | 8:62370401 | 42957 | 8:62370423 | 42960 | 8:62476415 | 42961 | 8:62476489 | 42962 | 8:62476593 |
| 42963 | 8:62481551 | 42964 | 8:62481563 | 42965 | 8:62492081 | 42966 | 8:62492171 | 42967 | 8:62492207 |
| 42969 | 8:62554727 | 42970 | 8:62556949 | 42977 | 8:62779236 | 42980 | 8:62823371 | 42985 | 8:62978731 |
| 42986 | 8:62978820 | 42989 | 8:63076318 | 42991 | 8:63182958 | 42998 | 8:63428149 | 43008 | 8:63577043 |
| 43025 | 8:64001632 | 43026 | 8:64003121 | 43031 | 8:64111218 | 43038 | 8:64443505 | 43041 | 8:64480556 |
| 43042 | 8:64512625 | 43050 | 8:64890322 | 43059 | 8:65128134 | 43060 | 8:65147458 | 43063 | 8:65217066 |
| 43064 | 8:65219496 | 43065 | 8:65219567 | 43073 | 8:65486798 | 43074 | 8:65486879 | 43075 | 8:65486964 |
| 43076 | 8:65487030 | 43077 | 8:65487045 | 43078 | 8:65487167 | 43079 | 8:65487520 | 43080 | 8:65506614 |
| 43092 | 8:66005380 | 43094 | 8:66041697 | 43095 | 8:66071027 | 43096 | 8:66071753 | 43099 | 8:66095067 |
| 43100 | 8:66095107 | 43102 | 8:66110177 | 43103 | 8:66110963 | 43104 | 8:66111005 | 43117 | 8:66467235 |
| 43129 | 8:66780866 | 43130 | 8:66781633 | 43132 | 8:66784915 | 43134 | 8:66833433 | 43135 | 8:66833492 |
| 43139 | 8:66987197 | 43140 | 8:66999877 | 43141 | 8:67000007 | 43143 | 8:67013017 | 43146 | 8:67072138 |
| 43148 | 8:67105385 | 43149 | 8:67105408 | 43150 | 8:67121705 | 43153 | 8:67145134 | 43154 | 8:67154528 |
| 43158 | 8:67392786 | 43162 | 8:67566051 | 43164 | 8:67576272 | 43166 | 8:67607555 | 43167 | 8:67613943 |
| 43170 | 8:67704512 | 43171 | 8:67731230 | 43182 | 8:67844754 | 43183 | 8:67844773 | 43200 | 8:68399674 |
| 43216 | 8:68844096 | 43217 | 8:68866548 | 43218 | 8:68866614 | 43223 | 8:68960840 | 43224 | 8:69009944 |
| 43227 | 8:69053764 | 43236 | 8:69156564 | 43237 | 8:69156580 | 43239 | 8:69252951 | 43241 | 8:69325956 |
| 43242 | 8:69421607 | 43258 | 8:69882196 | 43261 | 8:69947806 | 43262 | 8:69974129 | 43263 | 8:70014064 |
| 43264 | 8:70029487 | 43265 | 8:70039617 | 43280 | 8:70405010 | 43286 | 8:70466364 | 43288 | 8:70482064 |
| 43289 | 8:70489717 | 43290 | 8:70492869 | 43292 | 8:70501163 | 43297 | 8:70592634 | 43303 | 8:70815570 |
| 43306 | 8:70893052 | 43310 | 8:70958608 | 43319 | 8:71238004 | 43336 | 8:71880675 | 43360 | 8:72468833 |
| 43361 | 8:72468916 | 43362 | 8:72515403 | 43364 | 8:72551579 | 43366 | 8:72558376 | 43367 | 8:72561704 |
| 43372 | 8:72825901 | 43373 | 8:72828377 | 43375 | 8:72837275 | 43376 | 8:72844608 | 43377 | 8:72845970 |
| 43378 | 8:72904078 | 43380 | 8:72930313 | 43404 | 8:73494061 | 43406 | 8:73515301 | 43412 | 8:73656442 |
| 43423 | 8:73915262 | 43432 | 8:74168959 | 43436 | 8:74325878 | 43440 | 8:74465417 | 43444 | 8:74553402 |
| 43445 | 8:74558074 | 43446 | 8:74558704 | 43452 | 8:74712678 | 43458 | 8:74817911 | 43462 | 8:74863602 |
| 43463 | 8:74883099 | 43467 | 8:75123238 | 43471 | 8:75268745 | 43477 | 8:75477646 | 43481 | 8:75562796 |
| 43484 | 8:75700129 | 43487 | 8:75740079 | 43490 | 8:75853292 | 43497 | 8:76175098 | 43507 | 8:76433813 |
| 43508 | 8:76434785 | 43509 | 8:76434815 | 43511 | 8:76464537 | 43513 | 8:76492709 | 43515 | 8:76515741 |
| 43521 | 8:76664342 | 43522 | 8:76681835 | 43523 | 8:76684305 | 43524 | 8:76687450 | 43525 | 8:76703859 |
| 43526 | 8:76709352 | 43529 | 8:76742010 | 43530 | 8:76752971 | 43531 | 8:76754631 | 43532 | 8:76760562 |
| 43533 | 8:76762856 | 43534 | 8:76764812 | 43535 | 8:76768935 | 43538 | 8:76828964 | 43539 | 8:76835585 |
| 43540 | 8:76839168 | 43541 | 8:76841937 | 43542 | 8:76843765 | 43543 | 8:76844513 | 43544 | 8:76846849 |
| 43545 | 8:76850281 | 43546 | 8:76854581 | 43547 | 8:76870958 | 43548 | 8:76873029 | 43551 | 8:76975704 |
| 43553 | 8:76993039 | 43554 | 8:77006329 | 43556 | 8:77136545 | 43564 | 8:77562434 | 43581 | 8:78166070 |
| 43589 | 8:78358551 | 43595 | 8:78556780 | 43616 | 8:79447931 | 43642 | 8:80268872 | 43648 | 8:80405770 |
| 43652 | 8:80565130 | 43656 | 8:80613713 | 43660 | 8:80836273 | 43669 | 8:80974113 | 43686 | 8:81278385 |
| 43692 | 8:81389738 | 43695 | 8:81454715 | 43696 | 8:81454750 | 43698 | 8:81487284 | 43699 | 8:81487295 |
| 43704 | 8:81616321 | 43712 | 8:81744964 | 43716 | 8:81860516 | 43727 | 8:82113238 | 43728 | 8:82116405 |
| 43730 | 8:82160329 | 43751 | 8:82676827 | 43752 | 8:82677581 | 43753 | 8:82677988 | 43759 | 8:82823172 |
| 43762 | 8:82865703 | 43769 | 8:83085781 | 43782 | 8:83515046 | 43790 | 8:83738651 | 43794 | 8:83793151 |
| 43795 | 8:83793154 | 43796 | 8:83794591 | 43797 | 8:83794760 | 43799 | 8:83842360 | 43803 | 8:83879316 |
| 43808 | 8:83976437 | 43809 | 8:84045184 | 43811 | 8:84100704 | 43812 | 8:84123211 | 43813 | 8:84134245 |
| 43815 | 8:84172945 | 43829 | 8:84494416 | 43835 | 8:84811346 | 43842 | 8:84912804 | 43858 | 8:85366261 |
| 43873 | 8:85725330 | 43875 | 8:85804392 | 43884 | 8:85987078 | 43888 | 8:86079018 | 43891 | 8:86115895 |
| 43892 | 8:86133024 | 43895 | 8:86219410 | 43896 | 8:86226370 | 43905 | 8:86537816 | 43912 | 8:86719772 |
| 43914 | 8:86756176 | 43916 | 8:86779557 | 43917 | 8:86793653 | 43920 | 8:86935675 | 43921 | 8:86961975 |
| 43949 | 8:87523043 | 43955 | 8:87701857 | 43967 | 8:88033337 | 43968 | 8:88126576 | 43977 | 8:88363375 |
| 43986 | 8:88562919 | 43987 | 8:88572133 | 43995 | 8:88712829 | 43997 | 8:88762685 | 44001 | 8:88829394 |
| 44007 | 8:88990195 | 44013 | 8:89098414 | 44014 | 8:89113184 | 44016 | 8:89140453 | 44018 | 8:89141946 |
| 44019 | 8:89141971 | 44020 | 8:89142000 | 44021 | 8:89142063 | 44024 | 8:89269473 | 44033 | 8:89533107 |
| 44034 | 8:89533116 | 44035 | 8:89533121 | 44037 | 8:89557638 | 44041 | 8:89571402 | 44046 | 8:89722190 |
| 44049 | 8:89770693 | 44056 | 8:89926318 | 44059 | 8:90070089 | 44062 | 8:90193893 | 44084 | 8:90853473 |
| 44085 | 8:90853557 | 44086 | 8:90853588 | 44087 | 8:90853599 | 44101 | 8:91152034 | 44102 | 8:91161810 |
| 44106 | 8:91289222 | 44110 | 8:91381933 | 44111 | 8:91382574 | 44129 | 8:91948504 | 44137 | 8:91989858 |
| 44138 | 8:91995185 | 44143 | 8:92008855 | 44149 | 8:92136613 | 44168 | 8:92747513 | 44178 | 8:93068614 |
| 44179 | 8:93068725 | 44204 | 8:93841029 | 44208 | 8:94082045 | 44212 | 8:94260204 | 44220 | 8:94620469 |
| 44221 | 8:94620534 | 44222 | 8:94621015 | 44223 | 8:94623988 | 44224 | 8:94624900 | 44225 | 8:94672379 |
| 44226 | 8:94690978 | 44232 | 8:94939320 | 44241 | 8:95337173 | 44259 | 8:95796796 | 44263 | 8:95951526 |
| 44264 | 8:95977921 | 44266 | 8:96012156 | 44272 | 8:96165982 | 44273 | 8:96193360 | 44281 | 8:96384779 |
| 44282 | 8:96445578 | 44289 | 8:96573262 | 44308 | 8:97157793 | 44317 | 8:97299078 | 44319 | 8:97368811 |
| 44326 | 8:97495202 | 44327 | 8:97557161 | 44329 | 8:97577094 | 44330 | 8:97601680 | 44335 | 8:97795768 |
| 44350 | 8:98245729 | 44360 | 8:98592583 | 44362 | 8:98625126 | 44367 | 8:98741152 | 44368 | 8:98785042 |
| 44370 | 8:98829420 | 44375 | 8:98919680 | 44386 | 8:99142994 | 44392 | 8:99207386 | 44397 | 8:99291886 |
| 44400 | 8:99333533 | 44404 | 8:99388416 | 44405 | 8:99406177 | 44407 | 8:99412670 | 44409 | 8:99476683 |
| 44413 | 8:99525142 | 44415 | 8:99551509 | 44425 | 8:99654305 | 44429 | 8:99703737 | 44435 | 8:99959755 |
| 44436 | 8:99982505 | 44437 | 8:100010762 | 44446 | 8:100374163 | 44448 | 8:100401670 | 44449 | 8:100409615 |
| 44453 | 8:100428898 | 44455 | 8:100470224 | 44466 | 8:100654637 | 44471 | 8:100832272 | 44472 | 8:100876059 |
| 44478 | 8:101026237 | 44482 | 8:101247151 | 44485 | 8:101301768 | 44486 | 8:101312150 | 44494 | 8:101441924 |
| 44496 | 8:101485459 | 44500 | 8:101643451 | 44502 | 8:101701776 | 44506 | 8:101779830 | 44507 | 8:101800844 |
| 44517 | 8:102113941 | 44523 | 8:102234774 | 44525 | 8:102290809 | 44526 | 8:102291109 | 44527 | 8:102291157 |
| 44528 | 8:102291228 | 44529 | 8:102291230 | 44530 | 8:102291256 | 44531 | 8:102291263 | 44532 | 8:102293933 |
| 44533 | 8:102293974 | 44534 | 8:102299598 | 44535 | 8:102299601 | 44536 | 8:102305536 | 44538 | 8:102377736 |
| 44540 | 8:102402762 | 44541 | 8:102408863 | 44542 | 8:102411405 | 44546 | 8:102434252 | 44554 | 8:102594527 |
| 44555 | 8:102594773 | 44556 | 8:102595590 | 44557 | 8:102617174 | 44558 | 8:102618622 | 44560 | 8:102626997 |
| 44561 | 8:102634985 | 44562 | 8:102640018 | 44564 | 8:102642339 | 44566 | 8:102645680 | 44567 | 8:102648250 |
| 44568 | 8:102648741 | 44577 | 8:102834472 | 44579 | 8:102889304 | 44583 | 8:102931846 | 44596 | 8:103307658 |
| 44597 | 8:103316620 | 44598 | 8:103323065 | 44599 | 8:103323527 | 44600 | 8:103415237 | 44601 | 8:103442278 |
| 44602 | 8:103449094 | 44604 | 8:103484976 | 44605 | 8:103489848 | 44606 | 8:103500362 | 44607 | 8:103516850 |
| 44608 | 8:103529815 | 44609 | 8:103560463 | 44610 | 8:103572704 | 44611 | 8:103575282 | 44612 | 8:103580891 |
| 44613 | 8:103584228 | 44614 | 8:103588272 | 44615 | 8:103589059 | 44616 | 8:103591535 | 44618 | 8:103596979 |
| 44619 | 8:103598675 | 44620 | 8:103603170 | 44621 | 8:103612096 | 44622 | 8:103612794 | 44624 | 8:103623951 |
| 44625 | 8:103634947 | 44626 | 8:103640204 | 44627 | 8:103645354 | 44628 | 8:103649615 | 44630 | 8:103673828 |
| 44634 | 8:103783688 | 44646 | 8:104113096 | 44647 | 8:104114568 | 44650 | 8:104204807 | 44654 | 8:104251576 |
| 44655 | 8:104271837 | 44658 | 8:104292759 | 44659 | 8:104322730 | 44661 | 8:104414493 | 44663 | 8:104420746 |
| 44669 | 8:104546178 | 44674 | 8:104630353 | 44679 | 8:104760159 | 44681 | 8:104815425 | 44694 | 8:105160429 |
| 44716 | 8:105525844 | 44719 | 8:105592649 | 44722 | 8:105630176 | 44725 | 8:105696501 | 44727 | 8:105765759 |
| 44728 | 8:105774110 | 44732 | 8:105846896 | 44739 | 8:106082143 | 44744 | 8:106427938 | 44759 | 8:106936156 |
| 44760 | 8:106961448 | 44761 | 8:106981761 | 44762 | 8:107001526 | 44808 | 8:108127783 | 44829 | 8:108978247 |
| 44850 | 8:109853668 | 44855 | 8:110002325 | 44857 | 8:110059433 | 44860 | 8:110157715 | 44862 | 8:110244165 |
| 44863 | 8:110244337 | 44865 | 8:110248495 | 44866 | 8:110251397 | 44870 | 8:110445148 | 44871 | 8:110445202 |
| 44872 | 8:110445222 | 44874 | 8:110540868 | 44880 | 8:110749028 | 44882 | 8:110764849 | 44888 | 8:110972844 |
| 44890 | 8:110979223 | 44892 | 8:111002543 | 44893 | 8:111081452 | 44897 | 8:111284112 | 44932 | 9:349617 |
| 44935 | 9:449265 | 44965 | 9:761293 | 44966 | 9:761398 | 44970 | 9:946489 | 44983 | 9:1228475 |
| 44984 | 9:1228520 | 44985 | 9:1228587 | 44986 | 9:1228611 | 44987 | 9:1228669 | 44997 | 9:1530507 |
| 44998 | 9:1530528 | 45013 | 9:1926540 | 45016 | 9:2022731 | 45017 | 9:2022740 | 45084 | 9:4060756 |
| 45090 | 9:4309847 | 45101 | 9:4645222 | 45115 | 9:5086559 | 45159 | 9:6390476 | 45161 | 9:6450647 |
| 45163 | 9:6559884 | 45174 | 9:6887577 | 45189 | 9:7542350 | 45206 | 9:7865211 | 45219 | 9:8218235 |
| 45220 | 9:8218462 | 45223 | 9:8257979 | 45224 | 9:8258011 | 45225 | 9:8258047 | 45241 | 9:8662565 |
| 45242 | 9:8691938 | 45245 | 9:8740852 | 45246 | 9:8740922 | 45247 | 9:8741004 | 45255 | 9:9002672 |
| 45259 | 9:9073133 | 45265 | 9:9212931 | 45269 | 9:9363423 | 45281 | 9:9866377 | 45288 | 9:10026119 |
| 45290 | 9:10179658 | 45293 | 9:10229582 | 45295 | 9:10270107 | 45302 | 9:10376631 | 45307 | 9:10412914 |
| 45343 | 9:10650283 | 45377 | 9:10813074 | 45393 | 9:11379384 | 45398 | 9:11488885 | 45413 | 9:11816817 |
| 45418 | 9:11933227 | 45428 | 9:12091110 | 45431 | 9:12153363 | 45433 | 9:12204845 | 45435 | 9:12250871 |
| 45445 | 9:12499635 | 45446 | 9:12509673 | 45452 | 9:12645170 | 45471 | 9:12766564 | 45481 | 9:13100266 |
| 45485 | 9:13105495 | 45491 | 9:13132484 | 45496 | 9:13287682 | 45498 | 9:13361022 | 45503 | 9:13494908 |
| 45509 | 9:13684115 | 45510 | 9:13698663 | 45517 | 9:13880641 | 45520 | 9:13989518 | 45523 | 9:14095633 |
| 45524 | 9:14124920 | 45527 | 9:14203195 | 45540 | 9:14468437 | 45545 | 9:14564006 | 45548 | 9:14665440 |
| 45550 | 9:14717693 | 45564 | 9:15013572 | 45568 | 9:15038319 | 45570 | 9:15071350 | 45571 | 9:15081586 |
| 45574 | 9:15123580 | 45577 | 9:15163893 | 45578 | 9:15175648 | 45585 | 9:15369931 | 45587 | 9:15406354 |
| 45595 | 9:15497571 | 45596 | 9:15499363 | 45597 | 9:15499412 | 45598 | 9:15499535 | 45599 | 9:15511160 |
| 45602 | 9:15527205 | 45604 | 9:15548110 | 45605 | 9:15553535 | 45606 | 9:15558085 | 45607 | 9:15560500 |
| 45608 | 9:15560527 | 45609 | 9:15562142 | 45623 | 9:15687216 | 45631 | 9:15868930 | 45644 | 9:16138159 |
| 45653 | 9:16384844 | 45657 | 9:16537269 | 45674 | 9:17031734 | 45675 | 9:17031771 | 45687 | 9:17461271 |
| 45692 | 9:17595872 | 45717 | 9:18430308 | 45721 | 9:18529694 | 45722 | 9:18552564 | 45724 | 9:18645348 |
| 45726 | 9:18717619 | 45727 | 9:18731789 | 45729 | 9:18755368 | 45731 | 9:18791756 | 45732 | 9:18791819 |
| 45733 | 9:18791889 | 45738 | 9:18936509 | 45743 | 9:19046499 | 45745 | 9:19139676 | 45753 | 9:19435053 |
| 45776 | 9:20235228 | 45779 | 9:20303916 | 45816 | 9:21552246 | 45824 | 9:21653355 | 45833 | 9:21833987 |
| 45835 | 9:21852388 | 45840 | 9:21940817 | 45846 | 9:22080892 | 45848 | 9:22133199 | 45865 | 9:22573520 |
| 45867 | 9:22594113 | 45874 | 9:22795139 | 45879 | 9:22958243 | 45889 | 9:23151477 | 45890 | 9:23173931 |
| 45894 | 9:23218165 | 45898 | 9:23339249 | 45900 | 9:23430616 | 45901 | 9:23441779 | 45904 | 9:23456305 |
| 45906 | 9:23472649 | 45909 | 9:23546774 | 45915 | 9:23672358 | 45925 | 9:23857068 | 45932 | 9:24029631 |
| 45937 | 9:24145793 | 45943 | 9:24238183 | 45951 | 9:24426538 | 45970 | 9:25002484 | 45980 | 9:25350079 |
| 45981 | 9:25353185 | 45989 | 9:25530018 | 45997 | 9:25755576 | 45999 | 9:25785154 | 46003 | 9:25878075 |
| 46010 | 9:26028894 | 46015 | 9:26134547 | 46018 | 9:26208349 | 46028 | 9:26467397 | 46039 | 9:26838528 |
| 46040 | 9:26868103 | 46041 | 9:26898919 | 46048 | 9:27183994 | 46054 | 9:27249330 | 46057 | 9:27304567 |
| 46058 | 9:27317016 | 46061 | 9:27408248 | 46071 | 9:27587922 | 46077 | 9:27702582 | 46094 | 9:28136433 |
| 46108 | 9:28431328 | 46121 | 9:28644696 | 46127 | 9:28779206 | 46165 | 9:29391430 | 46177 | 9:29530654 |
| 46179 | 9:29544266 | 46183 | 9:29615614 | 46205 | 9:30230309 | 46207 | 9:30256998 | 46208 | 9:30257023 |
| 46209 | 9:30257052 | 46212 | 9:30289673 | 46219 | 9:30469930 | 46224 | 9:30615123 | 46227 | 9:30674435 |
| 46229 | 9:30739336 | 46237 | 9:30892065 | 46239 | 9:30932509 | 46240 | 9:30932519 | 46241 | 9:30932556 |
| 46247 | 9:31029451 | 46249 | 9:31141266 | 46252 | 9:31227971 | 46253 | 9:31227973 | 46255 | 9:31253066 |
| 46261 | 9:31394461 | 46271 | 9:31555978 | 46272 | 9:31588762 | 46273 | 9:31588794 | 46275 | 9:31725734 |
| 46283 | 9:31984894 | 46290 | 9:32052617 | 46291 | 9:32063888 | 46292 | 9:32063963 | 46296 | 9:32225638 |
| 46300 | 9:32284534 | 46301 | 9:32291884 | 46307 | 9:32437975 | 46308 | 9:32501496 | 46312 | 9:32638914 |
| 46315 | 9:32720871 | 46342 | 9:33372243 | 46349 | 9:33488593 | 46350 | 9:33496695 | 46356 | 9:33730807 |
| 46357 | 9:33736686 | 46358 | 9:33736719 | 46365 | 9:33922999 | 46376 | 9:34234529 | 46377 | 9:34239640 |
| 46383 | 9:34398858 | 46405 | 9:35059112 | 46422 | 9:35505083 | 46424 | 9:35630351 | 46428 | 9:35723297 |
| 46429 | 9:35723312 | 46430 | 9:35723316 | 46431 | 9:35723347 | 46432 | 9:35723412 | 46438 | 9:35923167 |
| 46445 | 9:36108895 | 46446 | 9:36118700 | 46448 | 9:36196365 | 46457 | 9:36321163 | 46464 | 9:36515253 |
| 46466 | 9:36617540 | 46468 | 9:36686271 | 46470 | 9:36765440 | 46475 | 9:36837540 | 46482 | 9:36952973 |
| 46485 | 9:36987967 | 46495 | 9:37162141 | 46500 | 9:37248499 | 46501 | 9:37262739 | 46504 | 9:37446666 |
| 46518 | 9:37786775 | 46532 | 9:38042996 | 46538 | 9:38200604 | 46540 | 9:38214307 | 46541 | 9:38222757 |
| 46542 | 9:38234690 | 46548 | 9:38345134 | 46549 | 9:38346604 | 46550 | 9:38349255 | 46551 | 9:38361398 |
| 46555 | 9:38439726 | 46556 | 9:38458789 | 46558 | 9:38513565 | 46560 | 9:38544090 | 46562 | 9:38561388 |
| 46568 | 9:38630128 | 46575 | 9:38725892 | 46578 | 9:38828299 | 46585 | 9:38890408 | 46589 | 9:38943133 |
| 46590 | 9:38963419 | 46603 | 9:39377711 | 46610 | 9:39610211 | 46611 | 9:39633208 | 46612 | 9:39637416 |
| 46613 | 9:39638258 | 46614 | 9:39639117 | 46616 | 9:39648286 | 46622 | 9:39764057 | 46623 | 9:39784094 |
| 46624 | 9:39793093 | 46627 | 9:39807347 | 46628 | 9:39807953 | 46629 | 9:39811555 | 46634 | 9:39934012 |
| 46635 | 9:39956055 | 46638 | 9:40007646 | 46646 | 9:40343063 | 46647 | 9:40346754 | 46649 | 9:40351241 |
| 46650 | 9:40353887 | 46651 | 9:40359510 | 46652 | 9:40394895 | 46653 | 9:40405455 | 46658 | 9:40559500 |
| 46664 | 9:40689518 | 46670 | 9:40792949 | 46673 | 9:40849838 | 46674 | 9:40851485 | 46675 | 9:40876197 |
| 46676 | 9:40920502 | 46677 | 9:40928750 | 46678 | 9:40936292 | 46679 | 9:40949947 | 46680 | 9:40960994 |
| 46681 | 9:40986746 | 46684 | 9:41057481 | 46690 | 9:41209197 | 46698 | 9:41519871 | 46702 | 9:41630405 |
| 46705 | 9:41713976 | 46712 | 9:42008068 | 46714 | 9:42049316 | 46715 | 9:42066260 | 46716 | 9:42073188 |
| 46721 | 9:42199260 | 46722 | 9:42202691 | 46723 | 9:42206745 | 46724 | 9:42207524 | 46726 | 9:42218456 |
| 46738 | 9:42539807 | 46741 | 9:42738988 | 46742 | 9:42751569 | 46743 | 9:42754079 | 46751 | 9:43079312 |
| 46752 | 9:43096509 | 46754 | 9:43117290 | 46755 | 9:43117292 | 46756 | 9:43117313 | 46757 | 9:43117336 |
| 46758 | 9:43117351 | 46759 | 9:43155466 | 46760 | 9:43155519 | 46761 | 9:43155650 | 46766 | 9:43352867 |
| 46779 | 9:43685269 | 46787 | 9:43948226 | 46803 | 9:44341413 | 46817 | 9:44599779 | 46820 | 9:44637804 |
| 46826 | 9:44854910 | 46842 | 9:45505888 | 46845 | 9:45550200 | 46868 | 9:46443428 | 46877 | 9:46667396 |
| 46883 | 9:46847687 | 46895 | 9:47304057 | 46923 | 9:48075628 | 46930 | 9:48336145 | 46939 | 9:48787417 |
| 46954 | 9:49224046 | 46955 | 9:49225376 | 46982 | 9:49910883 | 46995 | 9:50277864 | 47007 | 9:50567297 |
| 47008 | 9:50567298 | 47009 | 9:50567390 | 47012 | 9:50646985 | 47015 | 9:50694324 | 47019 | 9:50797460 |
| 47059 | 9:52046890 | 47080 | 9:52645687 | 47088 | 9:52787927 | 47093 | 9:52848517 | 47095 | 9:52861826 |
| 47120 | 9:53314350 | 47139 | 9:54071333 | 47143 | 9:54193058 | 47146 | 9:54279421 | 47153 | 9:54522488 |
| 47161 | 9:54675346 | 47181 | 9:55237255 | 47192 | 9:55526568 | 47193 | 9:55526660 | 47194 | 9:55529308 |
| 47228 | 9:56645296 | 47230 | 9:56687693 | 47234 | 9:56760440 | 47236 | 9:56787195 | 47245 | 9:57057433 |
| 47259 | 9:57574987 | 47260 | 9:57574995 | 47262 | 9:57634613 | 47272 | 9:57793857 | 47276 | 9:57849001 |
| 47284 | 9:58149441 | 47287 | 9:58311080 | 47288 | 9:58333810 | 47293 | 9:58457036 | 47301 | 9:58652669 |
| 47314 | 9:59159045 | 47351 | 9:59715217 | 47353 | 9:59771120 | 47355 | 9:59857183 | 47362 | 9:60072130 |
| 47364 | 9:60134972 | 47367 | 9:60247427 | 47369 | 9:60291706 | 47372 | 9:60358510 | 47377 | 9:60481368 |
| 47380 | 9:60536321 | 47383 | 9:60631920 | 47388 | 9:60745480 | 47393 | 9:60896389 | 47394 | 9:60903838 |
| 47397 | 9:60928250 | 47398 | 9:60928272 | 47400 | 9:60929060 | 47401 | 9:60930496 | 47402 | 9:60932205 |
| 47403 | 9:60936701 | 47406 | 9:61034023 | 47420 | 9:61625663 | 47428 | 9:61883689 | 47437 | 9:62153095 |
| 47438 | 9:62160605 | 47441 | 9:62239934 | 47444 | 9:62407115 | 47446 | 9:62441325 | 47448 | 9:62541808 |
| 47449 | 9:62541844 | 47450 | 9:62541925 | 47451 | 9:62569285 | 47453 | 9:62596000 | 47475 | 9:63213406 |
| 47481 | 9:63334601 | 47482 | 9:63334709 | 47483 | 9:63334737 | 47488 | 9:63564229 | 47490 | 9:63662723 |
| 47495 | 9:63879901 | 47505 | 9:64262650 | 47522 | 9:64653298 | 47523 | 9:64675425 | 47526 | 9:64806879 |
| 47527 | 9:64818979 | 47536 | 9:65014222 | 47537 | 9:65014886 | 47538 | 9:65015900 | 47539 | 9:65016625 |
| 47540 | 9:65017299 | 47541 | 9:65017970 | 47542 | 9:65018705 | 47543 | 9:65020386 | 47544 | 9:65022012 |
| 47545 | 9:65022960 | 47546 | 9:65024531 | 47547 | 9:65025971 | 47548 | 9:65026593 | 47549 | 9:65027537 |
| 47550 | 9:65029266 | 47551 | 9:65031641 | 47552 | 9:65033856 | 47553 | 9:65034695 | 47554 | 9:65037061 |
| 47555 | 9:65038847 | 47556 | 9:65039895 | 47557 | 9:65041060 | 47558 | 9:65041925 | 47559 | 9:65046945 |
| 47560 | 9:65048753 | 47561 | 9:65051331 | 47562 | 9:65052345 | 47564 | 9:65062111 | 47565 | 9:65063669 |
| 47566 | 9:65065713 | 47567 | 9:65069167 | 47568 | 9:65072452 | 47569 | 9:65074602 | 47570 | 9:65075729 |
| 47571 | 9:65076561 | 47572 | 9:65077195 | 47573 | 9:65077758 | 47574 | 9:65078630 | 47575 | 9:65118677 |
| 47576 | 9:65123323 | 47578 | 9:65179118 | 47579 | 9:65235666 | 47580 | 9:65243193 | 47581 | 9:65244259 |
| 47586 | 9:65383024 | 47589 | 9:65443065 | 47590 | 9:65446545 | 47591 | 9:65478206 | 47592 | 9:65483403 |
| 47598 | 9:65568211 | 47599 | 9:65570457 | 47600 | 9:65572272 | 47602 | 9:65604455 | 47609 | 9:65891235 |
| 47612 | 9:65971970 | 47615 | 9:66011528 | 47638 | 9:66597468 | 47648 | 9:66989120 | 47657 | 9:67225760 |
| 47663 | 9:67332887 | 47668 | 9:67459980 | 47681 | 9:67796936 | 47682 | 9:67823147 | 47686 | 9:67954748 |
| 47705 | 9:68370513 | 47708 | 9:68427995 | 47720 | 9:68912666 | 47721 | 9:68917528 | 47729 | 9:69060023 |
| 47730 | 9:69093647 | 47731 | 9:69104702 | 47732 | 9:69114502 | 47738 | 9:69297159 | 47741 | 9:69407676 |
| 47750 | 9:69583761 | 47751 | 9:69599853 | 47761 | 9:69896845 | 47767 | 9:70012144 | 47777 | 9:70295013 |
| 47779 | 9:70342326 | 47780 | 9:70347483 | 47786 | 9:70409386 | 47787 | 9:70409422 | 47789 | 9:70429923 |
| 47797 | 9:70751662 | 47798 | 9:70753720 | 47805 | 9:70887910 | 47809 | 9:71049805 | 47811 | 9:71060333 |
| 47813 | 9:71107122 | 47815 | 9:71116611 | 47820 | 9:71251152 | 47822 | 9:71292789 | 47824 | 9:71344053 |
| 47825 | 9:71356859 | 47826 | 9:71384334 | 47828 | 9:71407368 | 47829 | 9:71445600 | 47831 | 9:71492315 |
| 47834 | 9:71512303 | 47835 | 9:71513737 | 47840 | 9:71612772 | 47844 | 9:71651985 | 47848 | 9:71736589 |
| 47849 | 9:71761625 | 47864 | 9:72304039 | 47867 | 9:72384960 | 47877 | 9:72575284 | 47882 | 9:72722658 |
| 47883 | 9:72725124 | 47891 | 9:72888193 | 47892 | 9:72973276 | 47893 | 9:72989037 | 47895 | 9:73138567 |
| 47897 | 9:73175040 | 47902 | 9:73223596 | 47903 | 9:73244841 | 47915 | 9:73594271 | 47920 | 9:73701336 |
| 47921 | 9:73701377 | 47937 | 9:74031324 | 47939 | 9:74043724 | 47948 | 9:74315428 | 47955 | 9:74575135 |
| 47957 | 9:74617923 | 47959 | 9:74622543 | 47974 | 9:75094490 | 47975 | 9:75114725 | 47979 | 9:75261135 |
| 47984 | 9:75482552 | 47987 | 9:75505924 | 47991 | 9:75564777 | 47995 | 9:75627446 | 47999 | 9:75748546 |
| 48000 | 9:75749661 | 48001 | 9:75754806 | 48002 | 9:75754987 | 48014 | 9:76061643 | 48016 | 9:76103605 |
| 48022 | 9:76314846 | 48023 | 9:76319841 | 48038 | 9:76900400 | 48041 | 9:76954502 | 48042 | 9:76961448 |
| 48045 | 9:76972318 | 48048 | 9:77037622 | 48049 | 9:77047736 | 48050 | 9:77049274 | 48058 | 9:77255407 |
| 48063 | 9:77401724 | 48071 | 9:77497521 | 48150 | 9:78018701 | 48157 | 9:78383301 | 48163 | 9:78618960 |
| 48166 | 9:78653185 | 48173 | 9:78856788 | 48176 | 9:78984268 | 48192 | 9:79629564 | 48204 | 9:79900857 |
| 48206 | 9:79927447 | 48209 | 9:79992577 | 48211 | 9:79999363 | 48220 | 9:80143039 | 48222 | 9:80223447 |
| 48226 | 9:80281892 | 48235 | 9:80379126 | 48251 | 9:80913419 | 48252 | 9:80926042 | 48253 | 9:80927048 |
| 48254 | 9:80927101 | 48255 | 9:80935101 | 48256 | 9:80938115 | 48260 | 9:80960455 | 48281 | 9:81389583 |
| 48286 | 9:81502463 | 48289 | 9:81518292 | 48299 | 9:81745339 | 48304 | 9:81841021 | 48317 | 9:82097477 |
| 48318 | 9:82097512 | 48319 | 9:82100313 | 48337 | 9:82534749 | 48362 | 9:82854874 | 48364 | 9:82923714 |
| 48369 | 9:83119086 | 48374 | 9:83237503 | 48377 | 9:83309469 | 48391 | 9:83641203 | 48392 | 9:83672779 |
| 48399 | 9:83841026 | 48400 | 9:83857767 | 48418 | 9:84276446 | 48447 | 9:85144864 | 48452 | 9:85201593 |
| 48455 | 9:85222343 | 48457 | 9:85277103 | 48474 | 9:85565902 | 48476 | 9:85582831 | 48477 | 9:85582845 |
| 48478 | 9:85582856 | 48479 | 9:85582930 | 48485 | 9:85757204 | 48486 | 9:85777430 | 48487 | 9:85777435 |
| 48488 | 9:85777468 | 48489 | 9:85777555 | 48492 | 9:85801137 | 48494 | 9:85812059 | 48500 | 9:85926846 |
| 48507 | 9:86110491 | 48514 | 9:86329047 | 48532 | 9:86823737 | 48534 | 9:86935158 | 48537 | 9:86963116 |
| 48539 | 9:86989548 | 48541 | 9:87028872 | 48556 | 9:87348985 | 48561 | 9:87384465 | 48568 | 9:87494785 |
| 48570 | 9:87503445 | 48571 | 9:87504577 | 48572 | 9:87542588 | 48573 | 9:87544854 | 48593 | 9:87878916 |
| 48595 | 9:87890519 | 48596 | 9:87917511 | 48598 | 9:87929924 | 48599 | 9:87952953 | 48600 | 9:87962089 |
| 48601 | 9:87962734 | 48603 | 9:87966017 | 48604 | 9:87966917 | 48605 | 9:87975738 | 48610 | 9:88087724 |
| 48612 | 9:88140632 | 48613 | 9:88160188 | 48626 | 9:88569166 | 48628 | 9:88636603 | 48629 | 9:88636618 |
| 48630 | 9:88636634 | 48635 | 9:88732430 | 48644 | 9:88925002 | 48646 | 9:88943609 | 48654 | 9:89094468 |
| 48656 | 9:89166354 | 48658 | 9:89189099 | 48666 | 9:89326613 | 48667 | 9:89345202 | 48668 | 9:89346130 |
| 48670 | 9:89356829 | 48671 | 9:89373227 | 48672 | 9:89380245 | 48673 | 9:89387102 | 48674 | 9:89399914 |
| 48676 | 9:89410907 | 48677 | 9:89417136 | 48678 | 9:89423178 | 48680 | 9:89436296 | 48681 | 9:89443866 |
| 48682 | 9:89445782 | 48683 | 9:89445926 | 48684 | 9:89447805 | 48685 | 9:89454999 | 48688 | 9:89471837 |
| 48690 | 9:89496573 | 48698 | 9:89664367 | 48699 | 9:89708244 | 48700 | 9:89708807 | 48709 | 9:89919288 |
| 48733 | 9:90540018 | 48744 | 9:90795244 | 48745 | 9:90796860 | 48750 | 9:90844111 | 48751 | 9:90845595 |
| 48752 | 9:90845718 | 48762 | 9:91108815 | 48767 | 9:91144245 | 48776 | 9:91423091 | 48783 | 9:91591725 |
| 48784 | 9:91611257 | 48786 | 9:91670541 | 48789 | 9:91728675 | 48790 | 9:91734111 | 48794 | 9:91799932 |
| 48795 | 9:91808084 | 48802 | 9:92017600 | 48804 | 9:92051007 | 48808 | 9:92146096 | 48809 | 9:92173136 |
| 48823 | 9:92434720 | 48827 | 9:92618757 | 48831 | 9:92719541 | 48833 | 9:92726807 | 48834 | 9:92726908 |
| 48835 | 9:92732978 | 48844 | 9:92935922 | 48848 | 9:93101440 | 48860 | 9:93255745 | 48864 | 9:93329746 |
| 48865 | 9:93335995 | 48868 | 9:93434039 | 48869 | 9:93439818 | 48871 | 9:93544670 | 48872 | 9:93562913 |
| 48873 | 9:93583179 | 48874 | 9:93589365 | 48876 | 9:93593753 | 48878 | 9:93648481 | 48881 | 9:93756818 |
| 48882 | 9:93761851 | 48888 | 9:93864628 | 48899 | 9:94037282 | 48902 | 9:94059439 | 48903 | 9:94075786 |
| 48908 | 9:94239134 | 48909 | 9:94242901 | 48916 | 9:94279436 | 48925 | 9:94506574 | 48926 | 9:94511166 |
| 48928 | 9:94549002 | 48929 | 9:94552504 | 48930 | 9:94571641 | 48938 | 9:94770144 | 48943 | 9:94849176 |
| 48951 | 9:95011385 | 48952 | 9:95011440 | 48954 | 9:95075297 | 48957 | 9:95117095 | 48958 | 9:95119909 |
| 48959 | 9:95120912 | 48960 | 9:95120918 | 48961 | 9:95121102 | 48963 | 9:95137543 | 48965 | 9:95146949 |
| 48966 | 9:95149713 | 48967 | 9:95151479 | 48968 | 9:95151880 | 48969 | 9:95154305 | 48970 | 9:95159375 |
| 48971 | 9:95171473 | 48972 | 9:95239467 | 48975 | 9:95257977 | 48996 | 9:95960164 | 48997 | 9:95961292 |
| 48998 | 9:95964766 | 48999 | 9:95965222 | 49013 | 9:96262623 | 49016 | 9:96335077 | 49018 | 9:96352203 |
| 49020 | 9:96364316 | 49021 | 9:96365825 | 49023 | 9:96394133 | 49026 | 9:96454587 | 49027 | 9:96459992 |
| 49028 | 9:96460776 | 49029 | 9:96462068 | 49030 | 9:96464377 | 49031 | 9:96464974 | 49032 | 9:96465729 |
| 49033 | 9:96467828 | 49034 | 9:96471984 | 49035 | 9:96474365 | 49036 | 9:96475139 | 49037 | 9:96477053 |
| 49038 | 9:96478790 | 49039 | 9:96480951 | 49040 | 9:96482190 | 49041 | 9:96487368 | 49045 | 9:96575319 |
| 49047 | 9:96602946 | 49050 | 9:96636351 | 49064 | 9:96811911 | 49067 | 9:96862976 | 49068 | 9:96865435 |
| 49071 | 9:96875852 | 49072 | 9:96886282 | 49078 | 9:97002454 | 49079 | 9:97005408 | 49080 | 9:97006623 |
| 49107 | 9:97443586 | 49108 | 9:97443633 | 49112 | 9:97554130 | 49114 | 9:97571949 | 49117 | 9:97636004 |
| 49122 | 9:97688394 | 49125 | 9:97734208 | 49128 | 9:97781409 | 49129 | 9:97814093 | 49132 | 9:97896936 |
| 49140 | 9:97952202 | 49142 | 9:97987774 | 49146 | 9:98072257 | 49147 | 9:98090254 | 49149 | 9:98186273 |
| 49152 | 9:98271044 | 49162 | 9:98520046 | 49169 | 9:98643289 | 49173 | 9:98706036 | 49174 | 9:98706126 |
| 49177 | 9:98800653 | 49185 | 9:99049511 | 49197 | 9:99258284 | 49201 | 9:99320226 | 49202 | 9:99340641 |
| 49203 | 9:99363193 | 49205 | 9:99375752 | 49213 | 9:99564669 | 49223 | 9:99707846 | 49233 | 9:99913842 |
| 49238 | 9:100020368 | 49239 | 9:100037134 | 49248 | 9:100222700 | 49254 | 9:100288205 | 49264 | 9:100469286 |
| 49278 | 9:100659300 | 49294 | 9:101011242 | 49295 | 9:101027872 | 49296 | 9:101037743 | 49299 | 9:101107041 |
| 49302 | 9:101154740 | 49305 | 9:101206979 | 49311 | 9:101309902 | 49313 | 9:101329946 | 49314 | 9:101388778 |
| 49315 | 9:101404988 | 49320 | 9:101473236 | 49325 | 9:101520399 | 49330 | 9:101680647 | 49341 | 9:101799308 |
| 49351 | 9:101941022 | 49353 | 9:101969310 | 49354 | 9:101969389 | 49355 | 9:101970659 | 49356 | 9:101970691 |
| 49357 | 9:101971384 | 49358 | 9:101971424 | 49359 | 9:101976339 | 49360 | 9:101977590 | 49361 | 9:101977632 |
| 49362 | 9:101977719 | 49363 | 9:101977742 | 49364 | 9:101984019 | 49365 | 9:101984050 | 49366 | 9:101984250 |
| 49367 | 9:102004205 | 49368 | 9:102034495 | 49369 | 9:102034523 | 49370 | 9:102034580 | 49371 | 9:102034582 |
| 49373 | 9:102053353 | 49375 | 9:102065589 | 49376 | 9:102121235 | 49377 | 9:102127863 | 49380 | 9:102237624 |
| 49390 | 9:102348030 | 49391 | 9:102364770 | 49400 | 9:102589137 | 49402 | 9:102618152 | 49403 | 9:102619914 |
| 49404 | 9:102620509 | 49405 | 9:102622882 | 49409 | 9:102654533 | 49414 | 9:102687424 | 49416 | 9:102706741 |
| 49417 | 9:102710336 | 49422 | 9:102795356 | 49426 | 9:102898617 | 49427 | 9:102898674 | 49438 | 9:103153314 |
| 49439 | 9:103215814 | 49446 | 9:103329162 | 49447 | 9:103333336 | 49448 | 9:103336781 | 49457 | 9:103493999 |
| 49459 | 9:103575109 | 49460 | 9:103582734 | 49473 | 9:103926483 | 49474 | 9:103926928 | 49475 | 9:103927077 |
| 49476 | 9:103969118 | 49477 | 9:104003359 | 49485 | 9:104113993 | 49489 | 9:104355196 | 49490 | 9:104355303 |
| 49491 | 9:104356588 | 49492 | 9:104356685 | 49494 | 9:104376710 | 49501 | 10:84875 | 49519 | 10:695399 |
| 49526 | 10:769669 | 49544 | 10:1257872 | 49554 | 10:1412883 | 49570 | 10:1805121 | 49574 | 10:1814057 |
| 49576 | 10:1856119 | 49578 | 10:1881286 | 49579 | 10:1881323 | 49593 | 10:2147400 | 49602 | 10:2351254 |
| 49605 | 10:2373566 | 49626 | 10:2903794 | 49627 | 10:2903827 | 49629 | 10:2929612 | 49630 | 10:2932512 |
| 49647 | 10:3209297 | 49674 | 10:3744198 | 49675 | 10:3744214 | 49677 | 10:3772515 | 49678 | 10:3774094 |
| 49679 | 10:3802315 | 49691 | 10:4074788 | 49692 | 10:4104638 | 49705 | 10:4448386 | 49707 | 10:4468373 |
| 49709 | 10:4484450 | 49711 | 10:4554748 | 49716 | 10:4604295 | 49720 | 10:4638128 | 49726 | 10:4828321 |
| 49727 | 10:4829589 | 49732 | 10:5012847 | 49735 | 10:5111561 | 49755 | 10:5476055 | 49758 | 10:5506479 |
| 49775 | 10:5547565 | 49776 | 10:5550036 | 49777 | 10:5557416 | 49781 | 10:5609083 | 49797 | 10:6019241 |
| 49799 | 10:6059501 | 49802 | 10:6133685 | 49812 | 10:6396765 | 49816 | 10:6454997 | 49818 | 10:6473524 |
| 49820 | 10:6505409 | 49823 | 10:6590721 | 49826 | 10:6677707 | 49857 | 10:7516833 | 49859 | 10:7543493 |
| 49862 | 10:7600499 | 49873 | 10:7761962 | 49880 | 10:7901116 | 49884 | 10:8008379 | 49898 | 10:8326560 |
| 49905 | 10:8450920 | 49907 | 10:8478523 | 49911 | 10:8510644 | 49912 | 10:8510646 | 49914 | 10:8553549 |
| 49915 | 10:8553909 | 49920 | 10:8708761 | 49922 | 10:8777935 | 49926 | 10:8877008 | 49931 | 10:9028658 |
| 49934 | 10:9110701 | 49955 | 10:9671703 | 49956 | 10:9680242 | 49958 | 10:9697568 | 49963 | 10:9786271 |
| 49968 | 10:9804614 | 49979 | 10:10147987 | 49980 | 10:10172215 | 49983 | 10:10207865 | 49985 | 10:10220078 |
| 49990 | 10:10286771 | 49992 | 10:10393696 | 49993 | 10:10400749 | 49997 | 10:10519536 | 49999 | 10:10536820 |
| 50000 | 10:10537343 | 50001 | 10:10611527 | 50002 | 10:10633817 | 50005 | 10:10700036 | 50006 | 10:10706615 |
| 50007 | 10:10740773 | 50008 | 10:10740870 | 50009 | 10:10740872 | 50010 | 10:10742093 | 50012 | 10:10745459 |
| 50020 | 10:10855774 | 50025 | 10:10952491 | 50026 | 10:10983147 | 50028 | 10:10992296 | 50031 | 10:11037910 |
| 50037 | 10:11124874 | 50041 | 10:11194365 | 50042 | 10:11199580 | 50043 | 10:11214627 | 50044 | 10:11214653 |
| 50051 | 10:11459762 | 50052 | 10:11480188 | 50053 | 10:11484963 | 50054 | 10:11485435 | 50055 | 10:11533676 |
| 50062 | 10:11746501 | 50071 | 10:11984525 | 50072 | 10:11993432 | 50075 | 10:12037788 | 50077 | 10:12049463 |
| 50078 | 10:12049470 | 50080 | 10:12056720 | 50081 | 10:12058675 | 50082 | 10:12060333 | 50085 | 10:12193255 |
| 50087 | 10:12210976 | 50092 | 10:12363067 | 50096 | 10:12446219 | 50104 | 10:12563184 | 50108 | 10:12662259 |
| 50112 | 10:12776928 | 50118 | 10:13028042 | 50125 | 10:13148280 | 50129 | 10:13195695 | 50130 | 10:13201885 |
| 50137 | 10:13233050 | 50141 | 10:13251806 | 50146 | 10:13290512 | 50155 | 10:13433297 | 50159 | 10:13500606 |
| 50160 | 10:13521494 | 50163 | 10:13603249 | 50164 | 10:13604834 | 50184 | 10:13904093 | 50186 | 10:13947394 |
| 50187 | 10:13947449 | 50188 | 10:13953167 | 50191 | 10:14062089 | 50201 | 10:14318815 | 50203 | 10:14387176 |
| 50204 | 10:14387540 | 50216 | 10:14685085 | 50230 | 10:15045027 | 50231 | 10:15055444 | 50239 | 10:15212747 |
| 50241 | 10:15253352 | 50242 | 10:15253401 | 50244 | 10:15306720 | 50250 | 10:15382216 | 50251 | 10:15382286 |
| 50254 | 10:15491085 | 50261 | 10:15626947 | 50268 | 10:15765399 | 50269 | 10:15766293 | 50270 | 10:15766923 |
| 50271 | 10:15768048 | 50272 | 10:15777260 | 50274 | 10:15872387 | 50275 | 10:15872397 | 50276 | 10:15878690 |
| 50277 | 10:15887011 | 50278 | 10:15887086 | 50279 | 10:15888077 | 50280 | 10:15888157 | 50286 | 10:15986933 |
| 50287 | 10:15988001 | 50293 | 10:16160207 | 50294 | 10:16160289 | 50295 | 10:16160311 | 50300 | 10:16292572 |
| 50303 | 10:16351611 | 50309 | 10:16477102 | 50311 | 10:16505051 | 50315 | 10:16579579 | 50317 | 10:16615178 |
| 50318 | 10:16616715 | 50319 | 10:16622209 | 50321 | 10:16657010 | 50323 | 10:16689144 | 50325 | 10:16708640 |
| 50329 | 10:16760750 | 50330 | 10:16783563 | 50332 | 10:16840169 | 50333 | 10:16840192 | 50334 | 10:16840220 |
| 50346 | 10:17026997 | 50358 | 10:17327263 | 50362 | 10:17375623 | 50364 | 10:17405609 | 50372 | 10:17576175 |
| 50380 | 10:17746328 | 50393 | 10:18053005 | 50409 | 10:18204972 | 50413 | 10:18326097 | 50417 | 10:18459800 |
| 50429 | 10:18655176 | 50430 | 10:18656419 | 50440 | 10:19055444 | 50441 | 10:19055478 | 50444 | 10:19108089 |
| 50445 | 10:19108125 | 50446 | 10:19108146 | 50447 | 10:19108188 | 50448 | 10:19108578 | 50449 | 10:19108623 |
| 50450 | 10:19108629 | 50463 | 10:19296017 | 50473 | 10:19582204 | 50483 | 10:19757013 | 50484 | 10:19757066 |
| 50485 | 10:19757109 | 50492 | 10:19887391 | 50493 | 10:19887438 | 50494 | 10:19887463 | 50495 | 10:19887464 |
| 50496 | 10:19887506 | 50497 | 10:19906833 | 50502 | 10:20032774 | 50522 | 10:20511602 | 50523 | 10:20517856 |
| 50531 | 10:20716836 | 50533 | 10:20746648 | 50534 | 10:20749216 | 50535 | 10:20765182 | 50538 | 10:20798013 |
| 50539 | 10:20800521 | 50540 | 10:20823031 | 50542 | 10:20851467 | 50543 | 10:20854079 | 50545 | 10:20866367 |
| 50546 | 10:20866872 | 50547 | 10:20878828 | 50548 | 10:20887914 | 50549 | 10:20905201 | 50550 | 10:20905351 |
| 50551 | 10:20906045 | 50552 | 10:20922221 | 50555 | 10:20962445 | 50556 | 10:20969179 | 50557 | 10:20971568 |
| 50558 | 10:20980278 | 50559 | 10:20992105 | 50561 | 10:21041241 | 50564 | 10:21086459 | 50570 | 10:21193718 |
| 50581 | 10:21392700 | 50586 | 10:21482874 | 50587 | 10:21494322 | 50588 | 10:21496846 | 50590 | 10:21512555 |
| 50602 | 10:21928460 | 50604 | 10:21943044 | 50606 | 10:21954776 | 50607 | 10:21955205 | 50608 | 10:21961681 |
| 50609 | 10:21969630 | 50610 | 10:21969644 | 50611 | 10:21969862 | 50612 | 10:21970634 | 50613 | 10:21972554 |
| 50620 | 10:22271591 | 50629 | 10:22389791 | 50640 | 10:22796412 | 50644 | 10:23008980 | 50645 | 10:23058608 |
| 50685 | 10:25836315 | 50687 | 10:25842537 | 50689 | 10:25851554 | 50707 | 10:26370686 | 50712 | 10:26442221 |
| 50716 | 10:26521248 | 50719 | 10:26552273 | 50720 | 10:26552368 | 50725 | 10:26660705 | 50726 | 10:26664869 |
| 50728 | 10:26699741 | 50733 | 10:26735536 | 50739 | 10:26806421 | 50744 | 10:26852657 | 50747 | 10:26914900 |
| 50749 | 10:26936311 | 50751 | 10:26971279 | 50752 | 10:26979102 | 50753 | 10:26990817 | 50755 | 10:27045849 |
| 50756 | 10:27045893 | 50759 | 10:27104985 | 50760 | 10:27110316 | 50761 | 10:27110398 | 50767 | 10:27219754 |
| 50768 | 10:27219761 | 50770 | 10:27257251 | 50773 | 10:27354015 | 50774 | 10:27354260 | 50782 | 10:27629698 |
| 50786 | 10:27668710 | 50787 | 10:27668712 | 50788 | 10:27668749 | 50805 | 10:28336043 | 50818 | 10:28717710 |
| 50822 | 10:28777408 | 50823 | 10:28777482 | 50824 | 10:28777483 | 50825 | 10:28777517 | 50826 | 10:28816832 |
| 50828 | 10:28830145 | 50829 | 10:28830820 | 50830 | 10:28831951 | 50831 | 10:28837592 | 50834 | 10:28870874 |
| 50837 | 10:28920327 | 50838 | 10:28921627 | 50839 | 10:28922441 | 50841 | 10:28924088 | 50842 | 10:28924117 |
| 50846 | 10:28936523 | 50847 | 10:28936611 | 50849 | 10:28939012 | 50851 | 10:28941456 | 50852 | 10:28941564 |
| 50853 | 10:28941645 | 50854 | 10:28967860 | 50855 | 10:28967993 | 50865 | 10:29192994 | 50867 | 10:29207986 |
| 50875 | 10:29370574 | 50877 | 10:29394079 | 50879 | 10:29415078 | 50880 | 10:29434816 | 50883 | 10:29489538 |
| 50887 | 10:29551880 | 50894 | 10:29683783 | 50908 | 10:30087181 | 50918 | 10:30456072 | 50927 | 10:30882706 |
| 50929 | 10:31026079 | 50943 | 10:31383428 | 50944 | 10:31395880 | 50946 | 10:31421937 | 50948 | 10:31465645 |
| 50950 | 10:31539600 | 50965 | 10:31989230 | 50975 | 10:32311663 | 50987 | 10:32581894 | 50990 | 10:32646966 |
| 50993 | 10:32695364 | 50996 | 10:32766762 | 51016 | 10:33233733 | 51023 | 10:33473275 | 51025 | 10:33520438 |
| 51038 | 10:33775393 | 51040 | 10:33792624 | 51041 | 10:33803176 | 51052 | 10:34100470 | 51054 | 10:34214726 |
| 51056 | 10:34306045 | 51058 | 10:34339538 | 51062 | 10:34390466 | 51063 | 10:34399556 | 51065 | 10:34436191 |
| 51066 | 10:34441644 | 51067 | 10:34463264 | 51070 | 10:34502888 | 51078 | 10:34709456 | 51079 | 10:34711929 |
| 51084 | 10:34865113 | 51088 | 10:34924089 | 51101 | 10:35135295 | 51104 | 10:35165655 | 51105 | 10:35167815 |
| 51107 | 10:35180569 | 51109 | 10:35184337 | 51111 | 10:35210635 | 51112 | 10:35210945 | 51113 | 10:35213913 |
| 51114 | 10:35216558 | 51117 | 10:35370763 | 51128 | 10:35779938 | 51129 | 10:35856231 | 51131 | 10:35906682 |
| 51132 | 10:35916585 | 51135 | 10:35975249 | 51137 | 10:35982115 | 51143 | 10:36077032 | 51144 | 10:36131193 |
| 51151 | 10:36412382 | 51153 | 10:36441443 | 51154 | 10:36441686 | 51166 | 10:36836677 | 51170 | 10:36919070 |
| 51172 | 10:36945666 | 51173 | 10:36951207 | 51185 | 10:37239881 | 51188 | 10:37341433 | 51198 | 10:37638488 |
| 51212 | 10:37805187 | 51214 | 10:37904587 | 51218 | 10:37944601 | 51229 | 10:38333207 | 51231 | 10:38393603 |
| 51236 | 10:38488614 | 51245 | 10:38701360 | 51246 | 10:38753237 | 51267 | 10:39292295 | 51269 | 10:39363047 |
| 51271 | 10:39364510 | 51273 | 10:39377386 | 51303 | 10:40330913 | 51306 | 10:40373055 | 51307 | 10:40382252 |
| 51308 | 10:40389854 | 51309 | 10:40408498 | 51314 | 10:40556907 | 51321 | 10:40717249 | 51360 | 10:41919935 |
| 51369 | 10:42252004 | 51393 | 10:42819454 | 51397 | 10:42948816 | 51406 | 10:43236868 | 51413 | 10:43488771 |
| 51425 | 10:43985352 | 51432 | 10:44080128 | 51437 | 10:44252723 | 51446 | 10:44448699 | 51451 | 10:44583693 |
| 51456 | 10:44658796 | 51457 | 10:44687920 | 51470 | 10:44966040 | 51472 | 10:45022368 | 51476 | 10:45222091 |
| 51482 | 10:45334197 | 51483 | 10:45334223 | 51484 | 10:45334295 | 51485 | 10:45383083 | 51486 | 10:45385249 |
| 51487 | 10:45407393 | 51488 | 10:45421821 | 51492 | 10:45548786 | 51502 | 10:45782999 | 51504 | 10:45794472 |
| 51505 | 10:45797020 | 51507 | 10:45802351 | 51509 | 10:45807766 | 51510 | 10:45825756 | 51511 | 10:45825757 |
| 51512 | 10:45825760 | 51513 | 10:45852804 | 51515 | 10:45888410 | 51517 | 10:45962441 | 51535 | 10:46450572 |
| 51552 | 10:46711744 | 51556 | 10:46771999 | 51565 | 10:47008189 | 51572 | 10:47133365 | 51574 | 10:47141503 |
| 51576 | 10:47158682 | 51580 | 10:47240491 | 51585 | 10:47306453 | 51589 | 10:47384963 | 51590 | 10:47384967 |
| 51598 | 10:47600619 | 51601 | 10:47645531 | 51605 | 10:47674190 | 51608 | 10:47753050 | 51614 | 10:48040907 |
| 51632 | 10:48370815 | 51634 | 10:48417884 | 51644 | 10:48589378 | 51646 | 10:48589442 | 51648 | 10:48668493 |
| 51650 | 10:48743669 | 51660 | 10:49108347 | 51661 | 10:49132750 | 51668 | 10:49271034 | 51675 | 10:49350595 |
| 51677 | 10:49377221 | 51678 | 10:49377286 | 51681 | 10:49419982 | 51693 | 10:49601513 | 51703 | 10:49841256 |
| 51704 | 10:49841837 | 51705 | 10:49858694 | 51715 | 10:50107685 | 51734 | 10:50489426 | 51739 | 10:50557454 |
| 51740 | 10:50582984 | 51746 | 10:50846288 | 51749 | 10:51020894 | 51752 | 10:51062674 | 51757 | 10:51161988 |
| 51758 | 10:51163453 | 51761 | 10:51186753 | 51771 | 10:51429834 | 51772 | 10:51436172 | 51776 | 10:51594174 |
| 51786 | 10:51771324 | 51787 | 10:51772122 | 51798 | 10:52037658 | 51799 | 10:52053671 | 51807 | 10:52239885 |
| 51808 | 10:52247304 | 51818 | 10:52549691 | 51820 | 10:52577136 | 51821 | 10:52644574 | 51826 | 10:52759280 |
| 51827 | 10:52770881 | 51841 | 10:52889989 | 51843 | 10:52975439 | 51852 | 10:53392084 | 51855 | 10:53503163 |
| 51856 | 10:53566836 | 51857 | 10:53573889 | 51859 | 10:53599523 | 51870 | 10:54084338 | 51871 | 10:54097737 |
| 51873 | 10:54151043 | 51874 | 10:54166847 | 51879 | 10:54206436 | 51880 | 10:54223827 | 51885 | 10:54381985 |
| 51897 | 10:54587863 | 51900 | 10:54728840 | 51905 | 10:55049581 | 51908 | 10:55137727 | 51909 | 10:55138450 |
| 51915 | 10:55257378 | 51922 | 10:55519496 | 51928 | 10:55693450 | 51929 | 10:55701600 | 51930 | 10:55701734 |
| 51935 | 10:55824628 | 51937 | 10:55884610 | 51944 | 10:56050425 | 51950 | 10:56235551 | 51961 | 10:56404358 |
| 51974 | 10:56691062 | 52008 | 10:57738832 | 52009 | 10:57749028 | 52012 | 10:57825646 | 52021 | 10:58035128 |
| 52030 | 10:58215720 | 52031 | 10:58215742 | 52032 | 10:58223900 | 52033 | 10:58223933 | 52034 | 10:58223992 |
| 52038 | 10:58382992 | 52043 | 10:58575796 | 52048 | 10:58651910 | 52055 | 10:58892988 | 52058 | 10:58957454 |
| 52059 | 10:58957455 | 52060 | 10:58957462 | 52061 | 10:58963341 | 52068 | 10:59169515 | 52069 | 10:59257696 |
| 52070 | 10:59257779 | 52074 | 10:59431442 | 52084 | 10:59743414 | 52096 | 10:60229145 | 52099 | 10:60376158 |
| 52104 | 10:60467179 | 52132 | 10:61502794 | 52137 | 10:61691415 | 52142 | 10:61759883 | 52143 | 10:61761276 |
| 52144 | 10:61770176 | 52147 | 10:61801949 | 52152 | 10:61854226 | 52156 | 10:61927985 | 52161 | 10:62065140 |
| 52162 | 10:62070975 | 52167 | 10:62152633 | 52188 | 10:62478919 | 52189 | 10:62483660 | 52196 | 10:62841361 |
| 52197 | 10:62843919 | 52206 | 10:63003189 | 52207 | 10:63005018 | 52215 | 10:63176255 | 52217 | 10:63234235 |
| 52218 | 10:63236141 | 52230 | 10:63545031 | 52233 | 10:63676002 | 52235 | 10:63714642 | 52246 | 10:64098165 |
| 52254 | 10:64366724 | 52255 | 10:64366769 | 52256 | 10:64366784 | 52257 | 10:64366788 | 52258 | 10:64366808 |
| 52259 | 10:64366813 | 52260 | 10:64366843 | 52273 | 10:64623835 | 52274 | 10:64624712 | 52297 | 10:65311772 |
| 52298 | 10:65333523 | 52300 | 10:65360982 | 52301 | 10:65363558 | 52302 | 10:65366448 | 52303 | 10:65370682 |
| 52304 | 10:65374137 | 52305 | 10:65381281 | 52306 | 10:65388071 | 52307 | 10:65427744 | 52308 | 10:65428255 |
| 52309 | 10:65448723 | 52310 | 10:65460822 | 52311 | 10:65464926 | 52315 | 10:65667285 | 52317 | 10:65689392 |
| 52319 | 10:65693892 | 52320 | 10:65705467 | 52328 | 10:65975774 | 52333 | 10:66037974 | 52374 | 10:66920995 |
| 52379 | 10:67082677 | 52383 | 10:67193245 | 52384 | 10:67193602 | 52385 | 10:67207585 | 52388 | 10:67271539 |
| 52389 | 10:67275755 | 52400 | 10:67706373 | 52401 | 10:67706382 | 52403 | 10:67837002 | 52412 | 10:67919519 |
| 52415 | 10:68009703 | 52419 | 10:68069027 | 52426 | 10:68164248 | 52430 | 10:68213913 | 52434 | 10:68307915 |
| 52435 | 10:68336932 | 52445 | 10:68532314 | 52446 | 10:68532338 | 52449 | 10:68589455 | 52453 | 10:68616316 |
| 52454 | 10:68648835 | 52460 | 10:68845801 | 52479 | 10:69314335 | 52483 | 10:69414869 | 52489 | 10:69550619 |
| 52497 | 10:69813170 | 52506 | 10:69985799 | 52518 | 10:70311136 | 52530 | 10:70914151 | 52532 | 10:70933695 |
| 52543 | 10:71144557 | 52545 | 10:71190058 | 52547 | 10:71282860 | 52549 | 10:71395114 | 52550 | 10:71401946 |
| 52553 | 10:71492246 | 52560 | 10:71739567 | 52561 | 10:71739616 | 52562 | 10:71741589 | 52568 | 10:71990029 |
| 52594 | 10:72844951 | 52601 | 10:73180235 | 52603 | 10:73207644 | 52608 | 10:73381610 | 52619 | 10:73593430 |
| 52621 | 10:73621273 | 52625 | 10:73665667 | 52628 | 10:73702186 | 52631 | 10:73800772 | 52634 | 10:73889706 |
| 52639 | 10:74014694 | 52640 | 10:74014740 | 52647 | 10:74278788 | 52669 | 10:75144155 | 52684 | 10:75472993 |
| 52690 | 10:75648302 | 52702 | 10:76063641 | 52711 | 10:76208279 | 52712 | 10:76213571 | 52713 | 10:76216092 |
| 52714 | 10:76217503 | 52716 | 10:76219150 | 52717 | 10:76231514 | 52727 | 10:76445843 | 52739 | 10:76742309 |
| 52740 | 10:76780386 | 52750 | 10:76927979 | 52753 | 10:77027256 | 52754 | 10:77038645 | 52755 | 10:77043267 |
| 52756 | 10:77050549 | 52757 | 10:77063990 | 52758 | 10:77079521 | 52760 | 10:77086192 | 52761 | 10:77087944 |
| 52764 | 10:77165808 | 52765 | 10:77182957 | 52769 | 10:77284463 | 52770 | 10:77295759 | 52772 | 10:77310958 |
| 52783 | 10:77734390 | 52785 | 10:77815166 | 52787 | 10:77843400 | 52790 | 10:77908553 | 52794 | 10:77934474 |
| 52800 | 10:78024784 | 52804 | 10:78090692 | 52805 | 10:78091383 | 52806 | 10:78170657 | 52818 | 10:78466873 |
| 52819 | 10:78489026 | 52820 | 10:78524325 | 52833 | 10:78874750 | 52834 | 10:78890533 | 52843 | 10:79142128 |
| 52844 | 10:79142222 | 52850 | 10:79364696 | 52858 | 10:79552918 | 52864 | 10:79765450 | 52866 | 10:79778751 |
| 52876 | 10:80036481 | 52878 | 10:80058483 | 52887 | 10:80321841 | 52888 | 10:80354807 | 52890 | 10:80376119 |
| 52897 | 10:80516438 | 52898 | 10:80584353 | 52899 | 10:80612799 | 52908 | 10:80833008 | 52917 | 10:81113106 |
| 52918 | 10:81113113 | 52919 | 10:81113154 | 52920 | 10:81119799 | 52921 | 10:81122203 | 52922 | 10:81122225 |
| 52923 | 10:81133087 | 52924 | 10:81134221 | 52925 | 10:81141810 | 52926 | 10:81147906 | 52927 | 10:81159861 |
| 52928 | 10:81163746 | 52929 | 10:81169707 | 52930 | 10:81171404 | 52931 | 10:81174589 | 52932 | 10:81177743 |
| 52933 | 10:81179444 | 52934 | 10:81180179 | 52935 | 10:81182426 | 52936 | 10:81188153 | 52937 | 10:81191624 |
| 52938 | 10:81192985 | 52939 | 10:81201260 | 52942 | 10:81232104 | 52946 | 10:81275274 | 52948 | 10:81293569 |
| 52949 | 10:81313921 | 52954 | 10:81424405 | 52955 | 10:81429276 | 52956 | 10:81433859 | 52957 | 10:81434033 |
| 52960 | 10:81522739 | 52961 | 10:81563853 | 52971 | 10:81760421 | 52983 | 10:81884426 | 52985 | 10:81907059 |
| 52989 | 10:82020430 | 52991 | 10:82046798 | 52993 | 10:82113076 | 52994 | 10:82117432 | 52996 | 10:82155665 |
| 53001 | 10:82226992 | 53003 | 10:82262769 | 53010 | 10:82436819 | 53020 | 10:82813082 | 53031 | 10:83035771 |
| 53036 | 10:83168495 | 53037 | 10:83180678 | 53043 | 10:83268162 | 53064 | 10:83783323 | 53074 | 10:83967539 |
| 53078 | 10:84046423 | 53080 | 10:84074336 | 53081 | 10:84113189 | 53087 | 10:84235120 | 53088 | 10:84249302 |
| 53091 | 10:84274673 | 53093 | 10:84321770 | 53095 | 10:84358684 | 53098 | 10:84410074 | 53101 | 10:84432430 |
| 53113 | 10:84804439 | 53114 | 10:84809599 | 53117 | 10:84921371 | 53120 | 10:84937878 | 53122 | 10:84972916 |
| 53124 | 10:85090540 | 53125 | 10:85090775 | 53126 | 10:85093417 | 53129 | 10:85140109 | 53135 | 10:85210872 |
| 53139 | 10:85277645 | 53144 | 10:85348496 | 53148 | 10:85452360 | 53159 | 10:85646771 | 53161 | 10:85685359 |
| 53162 | 10:85714779 | 53163 | 10:85731082 | 53175 | 10:86198306 | 53179 | 10:86295239 | 53180 | 10:86322591 |
| 53182 | 10:86350641 | 53183 | 10:86373524 | 53186 | 10:86404676 | 53188 | 10:86427269 | 53191 | 10:86481134 |
| 53193 | 10:86492591 | 53194 | 10:86510757 | 53196 | 10:86545729 | 53197 | 10:86553401 | 53207 | 10:86743362 |
| 53209 | 10:86835796 | 53211 | 10:86863386 | 53214 | 10:86939644 | 53223 | 10:87113458 | 53244 | 10:87621927 |
| 53246 | 10:87627438 | 53250 | 10:87721184 | 53252 | 10:87755864 | 53256 | 10:87849396 | 53257 | 10:87849463 |
| 53258 | 10:87850909 | 53268 | 10:88187681 | 53270 | 10:88260182 | 53271 | 10:88298292 | 53274 | 10:88347395 |
| 53276 | 10:88380317 | 53280 | 10:88522805 | 53291 | 10:88839037 | 53292 | 10:88845164 | 53293 | 10:88846191 |
| 53297 | 10:88888537 | 53299 | 10:88925587 | 53313 | 10:89160492 | 53314 | 10:89160521 | 53316 | 10:89233565 |
| 53331 | 10:89626724 | 53332 | 10:89626819 | 53343 | 10:89798617 | 53348 | 10:89872058 | 53349 | 10:89872136 |
| 53350 | 10:89933077 | 53364 | 10:90186331 | 53378 | 10:90418700 | 53379 | 10:90418707 | 53415 | 10:90987301 |
| 53419 | 10:91011354 | 53420 | 10:91019729 | 53431 | 10:91348121 | 53444 | 10:91737619 | 53445 | 10:91746178 |
| 53449 | 10:91823694 | 53456 | 10:92080780 | 53458 | 10:92100804 | 53464 | 10:92232897 | 53465 | 10:92281587 |
| 53473 | 10:92466754 | 53476 | 10:92499113 | 53480 | 10:92594602 | 53482 | 10:92604984 | 53488 | 10:92710477 |
| 53489 | 10:92717162 | 53492 | 10:92799252 | 53507 | 10:93053048 | 53508 | 10:93053064 | 53512 | 10:93140659 |
| 53528 | 10:93626548 | 53537 | 10:93975527 | 53538 | 10:93977121 | 53549 | 10:94274383 | 53557 | 10:94377341 |
| 53558 | 10:94377386 | 53572 | 10:94695544 | 53576 | 10:94786454 | 53584 | 10:95064914 | 53595 | 10:95385183 |
| 53597 | 10:95412469 | 53602 | 10:95561991 | 53603 | 10:95575347 | 53608 | 10:95643374 | 53609 | 10:95672545 |
| 53611 | 10:95688501 | 53613 | 10:95728606 | 53618 | 10:95843138 | 53620 | 10:95843176 | 53628 | 10:96029745 |
| 53635 | 10:96240911 | 53638 | 10:96288699 | 53641 | 10:96380513 | 53642 | 10:96380599 | 53647 | 10:96569247 |
| 53650 | 10:96598589 | 53657 | 10:96839484 | 53659 | 10:96875821 | 53662 | 10:96976508 | 53673 | 10:97153819 |
| 53678 | 10:97206516 | 53683 | 10:97309948 | 53684 | 10:97309954 | 53692 | 10:97454671 | 53699 | 10:97550303 |
| 53700 | 10:97582963 | 53749 | 10:98562531 | 53777 | 10:99221264 | 53788 | 10:99540676 | 53789 | 10:99540722 |
| 53790 | 10:99540732 | 53791 | 10:99540816 | 53792 | 10:99540849 | 53805 | 10:99922832 | 53806 | 10:99926816 |
| 53808 | 10:99941485 | 53812 | 10:100010675 | 53815 | 10:100082394 | 53819 | 10:100180453 | 53820 | 10:100187779 |
| 53822 | 10:100243607 | 53825 | 10:100291362 | 53826 | 10:100301474 | 53827 | 10:100303380 | 53828 | 10:100303915 |
| 53829 | 10:100304470 | 53831 | 10:100330800 | 53852 | 10:100821682 | 53854 | 10:100876530 | 53858 | 10:100960737 |
| 53860 | 10:100963998 | 53861 | 10:100974311 | 53871 | 10:101130214 | 53872 | 10:101135621 | 53888 | 10:101435389 |
| 53889 | 10:101474959 | 53890 | 10:101488613 | 53891 | 10:101500216 | 53892 | 10:101506563 | 53903 | 10:101708370 |
| 53912 | 10:101805754 | 53918 | 10:101862805 | 53919 | 10:101870413 | 53924 | 10:102009836 | 53934 | 10:102135835 |
| 53937 | 10:102212274 | 53938 | 10:102220903 | 53940 | 10:102252835 | 53947 | 10:102361085 | 53950 | 10:102481713 |
| 53959 | 10:102713394 | 53962 | 10:102819206 | 54004 | 11:441458 | 54008 | 11:588104 | 54009 | 11:612918 |
| 54010 | 11:620469 | 54013 | 11:675675 | 54015 | 11:721258 | 54018 | 11:743177 | 54023 | 11:818693 |
| 54024 | 11:818745 | 54025 | 11:839682 | 54028 | 11:884789 | 54029 | 11:916613 | 54042 | 11:1140277 |
| 54051 | 11:1252661 | 54072 | 11:1599964 | 54073 | 11:1603522 | 54074 | 11:1604088 | 54076 | 11:1610503 |
| 54077 | 11:1613029 | 54078 | 11:1613069 | 54079 | 11:1615691 | 54080 | 11:1615825 | 54081 | 11:1615860 |
| 54082 | 11:1616045 | 54083 | 11:1617470 | 54084 | 11:1617491 | 54085 | 11:1618013 | 54088 | 11:1661775 |
| 54090 | 11:1692551 | 54091 | 11:1692581 | 54092 | 11:1692586 | 54094 | 11:1715577 | 54105 | 11:1929102 |
| 54106 | 11:1939516 | 54109 | 11:1975548 | 54112 | 11:2019047 | 54113 | 11:2019051 | 54116 | 11:2054152 |
| 54117 | 11:2058128 | 54122 | 11:2135119 | 54123 | 11:2165271 | 54124 | 11:2165552 | 54125 | 11:2165653 |
| 54135 | 11:2371904 | 54136 | 11:2375767 | 54137 | 11:2379791 | 54138 | 11:2380701 | 54139 | 11:2381359 |
| 54141 | 11:2381820 | 54142 | 11:2381938 | 54143 | 11:2382261 | 54144 | 11:2383322 | 54145 | 11:2383432 |
| 54147 | 11:2388794 | 54148 | 11:2391923 | 54149 | 11:2392633 | 54150 | 11:2392687 | 54151 | 11:2392744 |
| 54152 | 11:2393551 | 54153 | 11:2393601 | 54154 | 11:2393614 | 54155 | 11:2394563 | 54156 | 11:2394565 |
| 54157 | 11:2394754 | 54158 | 11:2429820 | 54159 | 11:2433742 | 54162 | 11:2466683 | 54163 | 11:2466765 |
| 54164 | 11:2466767 | 54176 | 11:2690324 | 54177 | 11:2691733 | 54179 | 11:2704089 | 54185 | 11:2887185 |
| 54186 | 11:2909443 | 54190 | 11:2965363 | 54191 | 11:2970198 | 54192 | 11:2971406 | 54193 | 11:2974009 |
| 54194 | 11:3048024 | 54195 | 11:3048066 | 54197 | 11:3068135 | 54198 | 11:3073771 | 54201 | 11:3080087 |
| 54202 | 11:3080140 | 54203 | 11:3080321 | 54204 | 11:3080329 | 54212 | 11:3324474 | 54214 | 11:3339436 |
| 54222 | 11:3496384 | 54223 | 11:3498026 | 54229 | 11:3630171 | 54232 | 11:3683284 | 54234 | 11:3687500 |
| 54235 | 11:3697811 | 54236 | 11:3703164 | 54241 | 11:3827658 | 54242 | 11:3831757 | 54243 | 11:3831764 |
| 54244 | 11:3833403 | 54245 | 11:3834470 | 54246 | 11:3834544 | 54249 | 11:3860402 | 54250 | 11:3861478 |
| 54251 | 11:3861495 | 54252 | 11:3863319 | 54258 | 11:4044668 | 54261 | 11:4144540 | 54274 | 11:4626193 |
| 54275 | 11:4643628 | 54277 | 11:4692004 | 54278 | 11:4715218 | 54280 | 11:4754025 | 54285 | 11:4846290 |
| 54288 | 11:4908167 | 54301 | 11:5122618 | 54306 | 11:5207458 | 54307 | 11:5208484 | 54308 | 11:5216864 |
| 54317 | 11:5475369 | 54320 | 11:5522837 | 54321 | 11:5546069 | 54322 | 11:5547645 | 54336 | 11:5892298 |
| 54338 | 11:5921461 | 54342 | 11:6014407 | 54344 | 11:6057385 | 54346 | 11:6090351 | 54353 | 11:6281176 |
| 54367 | 11:6672389 | 54371 | 11:6825527 | 54378 | 11:7079505 | 54379 | 11:7079550 | 54383 | 11:7226247 |
| 54385 | 11:7290568 | 54387 | 11:7312202 | 54390 | 11:7398056 | 54392 | 11:7442481 | 54395 | 11:7492967 |
| 54397 | 11:7506651 | 54406 | 11:7612707 | 54424 | 11:8016040 | 54441 | 11:8559109 | 54456 | 11:9088843 |
| 54457 | 11:9094665 | 54458 | 11:9108482 | 54459 | 11:9116906 | 54460 | 11:9122303 | 54461 | 11:9124299 |
| 54462 | 11:9125436 | 54463 | 11:9129918 | 54464 | 11:9140181 | 54465 | 11:9142056 | 54467 | 11:9146703 |
| 54468 | 11:9147988 | 54469 | 11:9149728 | 54470 | 11:9150188 | 54471 | 11:9150797 | 54472 | 11:9152234 |
| 54473 | 11:9153632 | 54477 | 11:9263561 | 54485 | 11:9432577 | 54493 | 11:9646962 | 54497 | 11:9717817 |
| 54499 | 11:9759593 | 54502 | 11:9782467 | 54509 | 11:9926241 | 54516 | 11:10139900 | 54517 | 11:10192082 |
| 54518 | 11:10205888 | 54519 | 11:10207241 | 54521 | 11:10218640 | 54522 | 11:10223170 | 54528 | 11:10378672 |
| 54534 | 11:10498811 | 54535 | 11:10506565 | 54539 | 11:10583951 | 54542 | 11:10706952 | 54543 | 11:10730447 |
| 54545 | 11:10770592 | 54547 | 11:10787415 | 54548 | 11:10804935 | 54549 | 11:10814646 | 54551 | 11:10817795 |
| 54559 | 11:11049635 | 54562 | 11:11215341 | 54564 | 11:11231984 | 54565 | 11:11242289 | 54573 | 11:11352441 |
| 54574 | 11:11362173 | 54576 | 11:11392077 | 54578 | 11:11435064 | 54584 | 11:11556298 | 54586 | 11:11616272 |
| 54601 | 11:12092660 | 54602 | 11:12096347 | 54608 | 11:12209379 | 54623 | 11:12569024 | 54625 | 11:12600200 |
| 54634 | 11:12790767 | 54643 | 11:13030163 | 54645 | 11:13087614 | 54648 | 11:13127507 | 54662 | 11:13410435 |
| 54667 | 11:13471629 | 54693 | 11:14069498 | 54697 | 11:14240512 | 54700 | 11:14312963 | 54707 | 11:14569196 |
| 54708 | 11:14601805 | 54717 | 11:14951793 | 54720 | 11:15049901 | 54729 | 11:15188618 | 54737 | 11:15351949 |
| 54800 | 11:16839124 | 54806 | 11:17005906 | 54808 | 11:17038164 | 54809 | 11:17038230 | 54811 | 11:17062733 |
| 54819 | 11:17340238 | 54820 | 11:17340240 | 54821 | 11:17361542 | 54823 | 11:17465587 | 54829 | 11:17736027 |
| 54830 | 11:17736142 | 54870 | 11:18811490 | 54871 | 11:18834617 | 54879 | 11:19016954 | 54885 | 11:19227167 |
| 54891 | 11:19479022 | 54893 | 11:19516116 | 54894 | 11:19516281 | 54897 | 11:19574521 | 54909 | 11:20009995 |
| 54914 | 11:20249271 | 54916 | 11:20268750 | 54929 | 11:20671302 | 54933 | 11:20761364 | 54935 | 11:20839310 |
| 54946 | 11:21100669 | 54948 | 11:21153847 | 54952 | 11:21298331 | 54953 | 11:21386222 | 54965 | 11:21549331 |
| 54968 | 11:21639708 | 54971 | 11:21679170 | 54979 | 11:21792131 | 54985 | 11:21911853 | 54998 | 11:22232061 |
| 54999 | 11:22277590 | 55009 | 11:22536318 | 55016 | 11:22632697 | 55031 | 11:23047670 | 55035 | 11:23109116 |
| 55043 | 11:23406318 | 55045 | 11:23420733 | 55057 | 11:23678147 | 55059 | 11:23713701 | 55060 | 11:23740579 |
| 55061 | 11:23792727 | 55062 | 11:23799914 | 55072 | 11:23927783 | 55073 | 11:23927808 | 55082 | 11:24309077 |
| 55114 | 11:24847692 | 55115 | 11:24847723 | 55120 | 11:25005279 | 55143 | 11:25650622 | 55144 | 11:25668988 |
| 55155 | 11:26000399 | 55158 | 11:26101828 | 55163 | 11:26155816 | 55171 | 11:26293504 | 55172 | 11:26294033 |
| 55175 | 11:26405127 | 55176 | 11:26406745 | 55179 | 11:26441367 | 55184 | 11:26597807 | 55202 | 11:27150645 |
| 55210 | 11:27309958 | 55223 | 11:27583040 | 55224 | 11:27612153 | 55225 | 11:27648326 | 55226 | 11:27664648 |
| 55227 | 11:27673338 | 55235 | 11:27863723 | 55243 | 11:28050164 | 55244 | 11:28054041 | 55254 | 11:28269292 |
| 55255 | 11:28312159 | 55256 | 11:28314792 | 55259 | 11:28347672 | 55261 | 11:28406003 | 55270 | 11:28648699 |
| 55271 | 11:28677976 | 55272 | 11:28697612 | 55275 | 11:28750175 | 55288 | 11:28998567 | 55292 | 11:29029776 |
| 55294 | 11:29094181 | 55305 | 11:29333853 | 55306 | 11:29339273 | 55311 | 11:29433875 | 55312 | 11:29446589 |
| 55314 | 11:29483268 | 55316 | 11:29523821 | 55320 | 11:29644262 | 55321 | 11:29652000 | 55328 | 11:29750755 |
| 55338 | 11:29944937 | 55339 | 11:30022269 | 55347 | 11:30283476 | 55357 | 11:30480608 | 55358 | 11:30487514 |
| 55359 | 11:30490953 | 55360 | 11:30498423 | 55363 | 11:30695691 | 55371 | 11:30816430 | 55375 | 11:30852665 |
| 55378 | 11:30909842 | 55381 | 11:31003265 | 55386 | 11:31096059 | 55394 | 11:31338792 | 55395 | 11:31338818 |
| 55396 | 11:31338832 | 55418 | 11:31989218 | 55419 | 11:32020262 | 55427 | 11:32309370 | 55440 | 11:32659947 |
| 55451 | 11:33038916 | 55454 | 11:33061320 | 55455 | 11:33063315 | 55465 | 11:33230608 | 55473 | 11:33483917 |
| 55497 | 11:34161881 | 55512 | 11:34992407 | 55515 | 11:35028247 | 55518 | 11:35081009 | 55523 | 11:35271551 |
| 55550 | 11:35973444 | 55555 | 11:36103640 | 55567 | 11:36415088 | 55575 | 11:36677962 | 55578 | 11:36729555 |
| 55579 | 11:36730226 | 55584 | 11:36935209 | 55590 | 11:37042393 | 55592 | 11:37065356 | 55593 | 11:37084548 |
| 55597 | 11:37151685 | 55602 | 11:37273838 | 55607 | 11:37321094 | 55610 | 11:37357247 | 55617 | 11:37555217 |
| 55622 | 11:37622807 | 55623 | 11:37622964 | 55629 | 11:37686699 | 55631 | 11:37699061 | 55632 | 11:37708915 |
| 55639 | 11:37993013 | 55640 | 11:38023147 | 55644 | 11:38167213 | 55652 | 11:38524637 | 55676 | 11:39146746 |
| 55698 | 11:39953399 | 55719 | 11:40780484 | 55720 | 11:40781022 | 55725 | 11:40977806 | 55729 | 11:41081305 |
| 55731 | 11:41095065 | 55732 | 11:41108333 | 55733 | 11:41112618 | 55734 | 11:41119005 | 55735 | 11:41119970 |
| 55737 | 11:41122253 | 55738 | 11:41130588 | 55739 | 11:41131422 | 55740 | 11:41138457 | 55742 | 11:41163401 |
| 55743 | 11:41165333 | 55744 | 11:41172319 | 55745 | 11:41190502 | 55746 | 11:41211212 | 55747 | 11:41236765 |
| 55757 | 11:41353565 | 55785 | 11:42015177 | 55813 | 11:42568364 | 55819 | 11:42700906 | 55838 | 11:43239984 |
| 55862 | 11:43925979 | 55864 | 11:44029565 | 55869 | 11:44191991 | 55875 | 11:44335233 | 55877 | 11:44398012 |
| 55884 | 11:44616476 | 55885 | 11:44669598 | 55886 | 11:44720675 | 55891 | 11:44864177 | 55892 | 11:44872149 |
| 55895 | 11:44907130 | 55896 | 11:44910831 | 55897 | 11:44915340 | 55899 | 11:44947747 | 55900 | 11:44960989 |
| 55901 | 11:44979580 | 55905 | 11:45085243 | 55910 | 11:45233285 | 55912 | 11:45240869 | 55926 | 11:45875224 |
| 55930 | 11:45909750 | 55932 | 11:45965043 | 55935 | 11:46005981 | 55939 | 11:46042064 | 55944 | 11:46118783 |
| 55948 | 11:46189212 | 55966 | 11:46586822 | 55984 | 11:46788369 | 55985 | 11:46796356 | 55986 | 11:46801200 |
| 55990 | 11:46887994 | 55991 | 11:46893010 | 55992 | 11:46895379 | 55993 | 11:46895384 | 55994 | 11:46895386 |
| 55995 | 11:46895443 | 55996 | 11:46896988 | 55997 | 11:46914387 | 55998 | 11:46915926 | 56001 | 11:46933431 |
| 56002 | 11:46946293 | 56009 | 11:47133868 | 56010 | 11:47136307 | 56012 | 11:47153161 | 56016 | 11:47318477 |
| 56020 | 11:47346660 | 56023 | 11:47402450 | 56025 | 11:47421701 | 56028 | 11:47484468 | 56039 | 11:47868107 |
| 56041 | 11:47957513 | 56051 | 11:48153310 | 56052 | 11:48179620 | 56055 | 11:48232533 | 56057 | 11:48284436 |
| 56064 | 11:48495828 | 56065 | 11:48519034 | 56066 | 11:48519745 | 56077 | 11:48624594 | 56078 | 11:48641618 |
| 56079 | 11:48643769 | 56080 | 11:48648683 | 56081 | 11:48652079 | 56082 | 11:48653016 | 56083 | 11:48653703 |
| 56084 | 11:48659221 | 56086 | 11:48661468 | 56087 | 11:48662095 | 56088 | 11:48663888 | 56089 | 11:48664547 |
| 56090 | 11:48665440 | 56091 | 11:48666103 | 56092 | 11:48666658 | 56093 | 11:48673397 | 56094 | 11:48673919 |
| 56095 | 11:48674533 | 56096 | 11:48675652 | 56097 | 11:48676148 | 56098 | 11:48677162 | 56099 | 11:48679806 |
| 56100 | 11:48680593 | 56101 | 11:48682804 | 56102 | 11:48685811 | 56103 | 11:48690618 | 56104 | 11:48695696 |
| 56105 | 11:48703362 | 56107 | 11:48712410 | 56108 | 11:48715777 | 56109 | 11:48729623 | 56110 | 11:48732364 |
| 56111 | 11:48740101 | 56112 | 11:48740643 | 56113 | 11:48742231 | 56114 | 11:48745355 | 56115 | 11:48748086 |
| 56116 | 11:48757634 | 56117 | 11:48788737 | 56118 | 11:48792299 | 56120 | 11:48910719 | 56134 | 11:49167307 |
| 56142 | 11:49394511 | 56144 | 11:49406127 | 56146 | 11:49419581 | 56151 | 11:49574160 | 56163 | 11:49832758 |
| 56164 | 11:49852553 | 56165 | 11:49852648 | 56200 | 11:51131191 | 56203 | 11:51184125 | 56220 | 11:51745809 |
| 56243 | 11:52697360 | 56265 | 11:53468115 | 56274 | 11:53833063 | 56290 | 11:54327571 | 56298 | 11:54566473 |
| 56300 | 11:54590112 | 56320 | 11:54776336 | 56323 | 11:54807232 | 56328 | 11:54930465 | 56329 | 11:54961590 |
| 56340 | 11:55186748 | 56347 | 11:55369267 | 56355 | 11:55552815 | 56356 | 11:55571320 | 56363 | 11:55683959 |
| 56364 | 11:55696252 | 56378 | 11:56190217 | 56402 | 11:56970533 | 56428 | 11:57877493 | 56439 | 11:58140156 |
| 56493 | 11:59622606 | 56497 | 11:59747111 | 56500 | 11:59861239 | 56510 | 11:60271069 | 56511 | 11:60271074 |
| 56512 | 11:60271117 | 56513 | 11:60271193 | 56520 | 11:60465745 | 56521 | 11:60465915 | 56522 | 11:60465920 |
| 56523 | 11:60465923 | 56524 | 11:60468952 | 56525 | 11:60470373 | 56526 | 11:60470388 | 56527 | 11:60471668 |
| 56529 | 11:60472081 | 56530 | 11:60474103 | 56531 | 11:60474134 | 56532 | 11:60474983 | 56533 | 11:60475508 |
| 56534 | 11:60475664 | 56535 | 11:60478021 | 56536 | 11:60478028 | 56537 | 11:60478049 | 56538 | 11:60478204 |
| 56540 | 11:60494769 | 56541 | 11:60494791 | 56542 | 11:60517676 | 56543 | 11:60566671 | 56544 | 11:60567347 |
| 56545 | 11:60567387 | 56546 | 11:60616886 | 56549 | 11:60641170 | 56550 | 11:60641182 | 56553 | 11:60671076 |
| 56557 | 11:60758092 | 56558 | 11:60775322 | 56560 | 11:60906972 | 56562 | 11:60949119 | 56564 | 11:60961745 |
| 56566 | 11:61011277 | 56567 | 11:61011512 | 56570 | 11:61079196 | 56573 | 11:61139067 | 56574 | 11:61149300 |
| 56575 | 11:61149340 | 56576 | 11:61149341 | 56578 | 11:61149398 | 56579 | 11:61149409 | 56581 | 11:61172809 |
| 56585 | 11:61361997 | 56588 | 11:61539166 | 56590 | 11:61548155 | 56606 | 11:62153933 | 56616 | 11:62410952 |
| 56621 | 11:62549111 | 56627 | 11:62639185 | 56629 | 11:62703391 | 56637 | 11:62848946 | 56645 | 11:63079035 |
| 56647 | 11:63132879 | 56657 | 11:63363010 | 56658 | 11:63376645 | 56661 | 11:63394494 | 56665 | 11:63456586 |
| 56671 | 11:63645957 | 56672 | 11:63664952 | 56676 | 11:63733057 | 56679 | 11:63799850 | 56687 | 11:63862724 |
| 56691 | 11:63954830 | 56693 | 11:63980633 | 56694 | 11:63980697 | 56695 | 11:64007710 | 56708 | 11:64345353 |
| 56712 | 11:64472503 | 56716 | 11:64576376 | 56718 | 11:64684536 | 56721 | 11:64807816 | 56726 | 11:64972478 |
| 56735 | 11:65170349 | 56736 | 11:65170393 | 56741 | 11:65341010 | 56743 | 11:65372142 | 56746 | 11:65438305 |
| 56766 | 11:65998851 | 56769 | 11:66070592 | 56775 | 11:66159407 | 56782 | 11:66307327 | 56783 | 11:66315381 |
| 56790 | 11:66473480 | 56795 | 11:66556801 | 56796 | 11:66556834 | 56799 | 11:66629962 | 56813 | 11:67084838 |
| 56814 | 11:67108786 | 56815 | 11:67114080 | 56821 | 11:67213238 | 56829 | 11:67439320 | 56832 | 11:67453087 |
| 56834 | 11:67479748 | 56835 | 11:67481439 | 56836 | 11:67483125 | 56838 | 11:67512057 | 56842 | 11:67544307 |
| 56843 | 11:67549232 | 56844 | 11:67549363 | 56846 | 11:67556514 | 56847 | 11:67563313 | 56848 | 11:67582936 |
| 56849 | 11:67582999 | 56850 | 11:67583014 | 56852 | 11:67588734 | 56855 | 11:67702445 | 56856 | 11:67709590 |
| 56857 | 11:67727467 | 56858 | 11:67728102 | 56859 | 11:67730113 | 56860 | 11:67736102 | 56861 | 11:67737073 |
| 56862 | 11:67747241 | 56863 | 11:67749685 | 56864 | 11:67752237 | 56865 | 11:67759745 | 56867 | 11:67762854 |
| 56868 | 11:67767430 | 56869 | 11:67772466 | 56870 | 11:67776969 | 56871 | 11:67787768 | 56872 | 11:67799383 |
| 56873 | 11:67805662 | 56874 | 11:67810598 | 56879 | 11:67896985 | 56882 | 11:67970890 | 56885 | 11:68111905 |
| 56893 | 11:68411391 | 56897 | 11:68467750 | 56909 | 11:68787390 | 56921 | 11:69071509 | 56932 | 11:69177619 |
| 56939 | 11:69288577 | 56941 | 11:69302814 | 56947 | 11:69479974 | 56950 | 11:69514045 | 56953 | 11:69570750 |
| 56954 | 11:69572243 | 56956 | 11:69619243 | 56957 | 11:69620020 | 56958 | 11:69620110 | 56960 | 11:69662827 |
| 56967 | 11:69745012 | 56992 | 11:70292588 | 56994 | 11:70341851 | 56995 | 11:70350355 | 57003 | 11:70595154 |
| 57010 | 11:70714155 | 57011 | 11:70718274 | 57013 | 11:70755284 | 57014 | 11:70761156 | 57015 | 11:70761179 |
| 57016 | 11:70761872 | 57017 | 11:70762170 | 57018 | 11:70762689 | 57019 | 11:70762700 | 57020 | 11:70762702 |
| 57021 | 11:70762781 | 57022 | 11:70762862 | 57023 | 11:70762868 | 57024 | 11:70763022 | 57025 | 11:70763076 |
| 57026 | 11:70763145 | 57027 | 11:70763553 | 57028 | 11:70763715 | 57029 | 11:70763906 | 57030 | 11:70764185 |
| 57031 | 11:70764539 | 57032 | 11:70768163 | 57033 | 11:70768281 | 57034 | 11:70769360 | 57035 | 11:70769405 |
| 57036 | 11:70775819 | 57037 | 11:70782767 | 57038 | 11:70782825 | 57039 | 11:70806849 | 57040 | 11:70807701 |
| 57041 | 11:70810781 | 57043 | 11:70862638 | 57048 | 11:71062901 | 57049 | 11:71062934 | 57053 | 11:71130818 |
| 57054 | 11:71131623 | 57057 | 11:71142342 | 57059 | 11:71149248 | 57066 | 11:71175312 | 57067 | 11:71177865 |
| 57068 | 11:71178203 | 57070 | 11:71182683 | 57072 | 11:71198214 | 57073 | 11:71199013 | 57074 | 11:71208751 |
| 57081 | 11:71398446 | 57094 | 11:71666080 | 57095 | 11:71713947 | 57099 | 11:71827640 | 57102 | 11:71961107 |
| 57103 | 11:71974375 | 57108 | 11:72101578 | 57112 | 11:72181248 | 57128 | 11:72810370 | 57133 | 11:72851670 |
| 57144 | 11:72988812 | 57146 | 11:73025557 | 57148 | 11:73068186 | 57150 | 11:73079672 | 57151 | 11:73079767 |
| 57152 | 11:73084466 | 57153 | 11:73087461 | 57154 | 11:73089558 | 57155 | 11:73090095 | 57158 | 11:73133312 |
| 57159 | 11:73148896 | 57161 | 11:73219332 | 57163 | 11:73277111 | 57166 | 11:73438358 | 57167 | 11:73480475 |
| 57177 | 11:73781192 | 57181 | 11:73853326 | 57185 | 11:73967619 | 57188 | 11:74009513 | 57190 | 11:74018063 |
| 57194 | 11:74054528 | 57205 | 11:74195149 | 57206 | 11:74204674 | 57207 | 11:74205573 | 57209 | 11:74245610 |
| 57213 | 11:74399369 | 57218 | 11:74544795 | 57227 | 11:74974483 | 57230 | 11:75017890 | 57232 | 11:75044406 |
| 57235 | 11:75106218 | 57236 | 11:75198521 | 57242 | 11:75328149 | 57243 | 11:75330425 | 57245 | 11:75334844 |
| 57246 | 11:75384323 | 57249 | 11:75420509 | 57250 | 11:75420581 | 57251 | 11:75477129 | 57252 | 11:75482256 |
| 57254 | 11:75493333 | 57256 | 11:75509869 | 57257 | 11:75513821 | 57266 | 11:75686867 | 57273 | 11:75929380 |
| 57280 | 11:76017351 | 57309 | 11:76876371 | 57310 | 11:76952531 | 57321 | 11:77091873 | 57322 | 11:77092007 |
| 57323 | 11:77121176 | 57324 | 11:77123614 | 57325 | 11:77124254 | 57327 | 11:77132776 | 57332 | 11:77262392 |
| 57355 | 11:77817486 | 57362 | 11:77896861 | 57363 | 11:77897300 | 57364 | 11:77897480 | 57365 | 11:77897481 |
| 57366 | 11:77897524 | 57367 | 11:77899208 | 57368 | 11:77899796 | 57369 | 11:77900345 | 57370 | 11:77900437 |
| 57371 | 11:77900488 | 57372 | 11:77900522 | 57373 | 11:77901876 | 57374 | 11:77901997 | 57375 | 11:77916472 |
| 57376 | 11:77916653 | 57377 | 11:77917793 | 57378 | 11:77918799 | 57379 | 11:77920101 | 57380 | 11:77920618 |
| 57381 | 11:77920740 | 57382 | 11:77920832 | 57384 | 11:77922179 | 57385 | 11:77922588 | 57386 | 11:77923067 |
| 57387 | 11:77927350 | 57398 | 11:78226756 | 57401 | 11:78268974 | 57402 | 11:78275050 | 57403 | 11:78303645 |
| 57407 | 11:78375724 | 57412 | 11:78529857 | 57415 | 11:78631628 | 57417 | 11:78663074 | 57419 | 11:78724728 |
| 57430 | 11:78997006 | 57438 | 11:79084041 | 57452 | 11:79229476 | 57454 | 11:79233862 | 57479 | 11:79764049 |
| 57489 | 11:80152318 | 57494 | 11:80309473 | 57496 | 11:80321806 | 57505 | 11:80474151 | 57507 | 11:80513212 |
| 57514 | 11:80648310 | 57515 | 11:80671943 | 57516 | 11:80690342 | 57545 | 11:81671201 | 57546 | 11:81671227 |
| 57551 | 11:81785970 | 57552 | 11:81800692 | 57558 | 11:81881809 | 57575 | 11:82260811 | 57580 | 11:82351733 |
| 57583 | 11:82364482 | 57591 | 11:82664554 | 57592 | 11:82664600 | 57593 | 11:82664609 | 57594 | 11:82664725 |
| 57596 | 11:82671761 | 57597 | 11:82677475 | 57598 | 11:82677728 | 57599 | 11:82683785 | 57600 | 11:82686842 |
| 57601 | 11:82687013 | 57602 | 11:82687072 | 57603 | 11:82687666 | 57604 | 11:82700336 | 57605 | 11:82709413 |
| 57621 | 11:83006401 | 57622 | 11:83027036 | 57623 | 11:83038638 | 57629 | 11:83118274 | 57630 | 11:83137062 |
| 57652 | 11:83676352 | 57692 | 11:84965273 | 57709 | 11:85278395 | 57711 | 11:85316120 | 57712 | 11:85350978 |
| 57720 | 11:85498225 | 57722 | 11:85595081 | 57731 | 11:85746062 | 57733 | 11:85771451 | 57736 | 11:85853743 |
| 57738 | 11:85936758 | 57748 | 11:86094641 | 57750 | 11:86113594 | 57753 | 11:86201573 | 57757 | 11:86221979 |
| 57758 | 11:86224683 | 57789 | 11:87198869 | 57790 | 11:87208683 | 57792 | 11:87231125 | 57797 | 11:87357375 |
| 57798 | 11:87380616 | 57803 | 11:87492469 | 57804 | 11:87494268 | 57805 | 11:87495906 | 57807 | 11:87520617 |
| 57808 | 11:87530939 | 57812 | 11:87650800 | 57815 | 11:87710317 | 57821 | 11:87813732 | 57823 | 11:87882552 |
| 57829 | 11:87970096 | 57830 | 11:87972654 | 57831 | 11:87972763 | 57832 | 11:87975372 | 57833 | 11:87980700 |
| 57834 | 11:87982062 | 57835 | 11:87982864 | 57836 | 11:87986573 | 57837 | 11:87987392 | 57838 | 11:87991576 |
| 57839 | 11:87992353 | 57840 | 11:87994077 | 57841 | 11:87994761 | 57842 | 11:87995976 | 57843 | 11:87996439 |
| 57844 | 11:87997041 | 57845 | 11:87997518 | 57846 | 11:87998024 | 57848 | 11:88000459 | 57849 | 11:88001097 |
| 57850 | 11:88002138 | 57851 | 11:88003290 | 57852 | 11:88006051 | 57853 | 11:88006580 | 57854 | 11:88007139 |
| 57855 | 11:88008201 | 57857 | 11:88009081 | 57858 | 11:88009883 | 57859 | 11:88011349 | 57860 | 11:88013047 |
| 57862 | 11:88020282 | 57863 | 11:88021880 | 57864 | 11:88030641 | 57866 | 11:88045066 | 57873 | 11:88105264 |
| 57885 | 11:88479108 | 57890 | 11:88733341 | 57897 | 11:88922785 | 57898 | 11:88922827 | 57899 | 11:88922860 |
| 57902 | 11:88976021 | 57904 | 11:88994635 | 57909 | 11:89132552 | 57910 | 11:89133642 | 57911 | 11:89152187 |
| 57912 | 11:89213759 | 57919 | 11:89375784 | 57920 | 11:89387726 | 57929 | 11:89561970 | 57940 | 11:89843767 |
| 57960 | 11:90408075 | 57965 | 11:90443507 | 57970 | 11:90503652 | 57977 | 11:90744044 | 57979 | 11:90785269 |
| 57980 | 11:90787434 | 57984 | 11:90906061 | 57989 | 11:90982703 | 57991 | 11:91054916 | 57994 | 11:91084013 |
| 58002 | 11:91266370 | 58006 | 11:91343800 | 58061 | 11:92402829 | 58062 | 11:92454234 | 58063 | 11:92473780 |
| 58064 | 11:92476182 | 58073 | 11:92661797 | 58074 | 11:92674766 | 58077 | 11:92715331 | 58084 | 11:92833095 |
| 58095 | 11:93210615 | 58098 | 11:93223406 | 58116 | 11:93629686 | 58119 | 11:93727304 | 58126 | 11:93972515 |
| 58127 | 11:93973132 | 58128 | 11:93973249 | 58129 | 11:93973432 | 58131 | 11:94067508 | 58132 | 11:94076855 |
| 58137 | 11:94178079 | 58140 | 11:94211102 | 58144 | 11:94339459 | 58148 | 11:94453289 | 58151 | 11:94500564 |
| 58152 | 11:94531181 | 58164 | 11:94880948 | 58173 | 11:95280882 | 58177 | 11:95315895 | 58178 | 11:95322263 |
| 58181 | 11:95425616 | 58190 | 11:95586651 | 58193 | 11:95692063 | 58201 | 11:95840093 | 58207 | 11:96117932 |
| 58209 | 11:96142116 | 58213 | 11:96261764 | 58215 | 11:96285921 | 58216 | 11:96322123 | 58217 | 11:96322228 |
| 58218 | 11:96338458 | 58230 | 11:96820150 | 58245 | 11:97196967 | 58248 | 11:97225356 | 58251 | 11:97254785 |
| 58253 | 11:97286893 | 58276 | 11:97843712 | 58277 | 11:97843818 | 58278 | 11:97883599 | 58279 | 11:97892319 |
| 58289 | 11:98162222 | 58290 | 11:98162477 | 58291 | 11:98173675 | 58292 | 11:98179996 | 58293 | 11:98189444 |
| 58294 | 11:98191816 | 58295 | 11:98193416 | 58298 | 11:98222190 | 58304 | 11:98396073 | 58313 | 11:98726635 |
| 58316 | 11:98813314 | 58321 | 11:98866430 | 58327 | 11:98921444 | 58328 | 11:98921481 | 58331 | 11:98929180 |
| 58332 | 11:98929534 | 58333 | 11:98931991 | 58334 | 11:98932587 | 58335 | 11:98951509 | 58337 | 11:98980770 |
| 58338 | 11:99034896 | 58339 | 11:99114771 | 58342 | 11:99223633 | 58343 | 11:99225289 | 58346 | 11:99292200 |
| 58347 | 11:99366886 | 58348 | 11:99391843 | 58349 | 11:99395118 | 58350 | 11:99395609 | 58351 | 11:99396169 |
| 58352 | 11:99397143 | 58353 | 11:99398436 | 58354 | 11:99399218 | 58355 | 11:99400709 | 58356 | 11:99401571 |
| 58357 | 11:99402294 | 58358 | 11:99403416 | 58359 | 11:99409144 | 58360 | 11:99424233 | 58361 | 11:99434036 |
| 58362 | 11:99444575 | 58368 | 11:99668885 | 58378 | 11:99888002 | 58382 | 11:99923257 | 58383 | 11:99932292 |
| 58384 | 11:99932294 | 58392 | 11:100028654 | 58402 | 11:100205236 | 58404 | 11:100238751 | 58405 | 11:100244628 |
| 58408 | 11:100346943 | 58413 | 11:100462505 | 58421 | 11:100658686 | 58423 | 11:100696639 | 58432 | 11:100894137 |
| 58433 | 11:100894144 | 58434 | 11:100894185 | 58435 | 11:100894200 | 58436 | 11:100894224 | 58437 | 11:100894236 |
| 58438 | 11:100895849 | 58439 | 11:100898629 | 58440 | 11:100899103 | 58441 | 11:100900308 | 58442 | 11:100900837 |
| 58443 | 11:100901391 | 58444 | 11:100902989 | 58445 | 11:100903015 | 58446 | 11:100903036 | 58447 | 11:100903107 |
| 58448 | 11:100904379 | 58449 | 11:100905484 | 58450 | 11:100905522 | 58451 | 11:100905593 | 58452 | 11:100920037 |
| 58453 | 11:100920185 | 58455 | 11:100929482 | 58456 | 11:100929917 | 58465 | 11:101211138 | 58469 | 11:101395761 |
| 58481 | 11:101767662 | 58482 | 11:101791346 | 58490 | 11:101950257 | 58498 | 11:102165298 | 58503 | 11:102356696 |
| 58505 | 11:102369158 | 58507 | 11:102451127 | 58508 | 11:102455536 | 58509 | 11:102455618 | 58510 | 11:102455647 |
| 58512 | 11:102480403 | 58520 | 11:102603145 | 58521 | 11:102603182 | 58527 | 11:102734173 | 58534 | 11:102808918 |
| 58535 | 11:102822945 | 58537 | 11:102847045 | 58539 | 11:102874693 | 58540 | 11:102878251 | 58541 | 11:102878949 |
| 58542 | 11:102883187 | 58543 | 11:102887734 | 58544 | 11:102893215 | 58545 | 11:102894321 | 58546 | 11:102895120 |
| 58547 | 11:102896098 | 58548 | 11:102896671 | 58549 | 11:102898861 | 58550 | 11:102899743 | 58551 | 11:102900707 |
| 58552 | 11:102900970 | 58553 | 11:102903216 | 58555 | 11:102937242 | 58561 | 11:103031296 | 58563 | 11:103113742 |
| 58565 | 11:103127844 | 58570 | 11:103259118 | 58571 | 11:103259131 | 58572 | 11:103259143 | 58573 | 11:103259232 |
| 58574 | 11:103273008 | 58580 | 11:103375332 | 58582 | 11:103380279 | 58586 | 11:103426894 | 58599 | 11:103765434 |
| 58603 | 11:103894196 | 58610 | 11:104163269 | 58611 | 11:104163320 | 58614 | 11:104188357 | 58616 | 11:104207359 |
| 58621 | 11:104247061 | 58626 | 11:104405742 | 58641 | 11:104665123 | 58647 | 11:104717230 | 58648 | 11:104722406 |
| 58664 | 11:104997566 | 58666 | 11:105082281 | 58669 | 11:105149212 | 58675 | 11:105246638 | 58676 | 11:105267690 |
| 58678 | 11:105368522 | 58679 | 11:105368628 | 58682 | 11:105496672 | 58683 | 11:105565466 | 58684 | 11:105565480 |
| 58685 | 11:105565571 | 58686 | 11:105586479 | 58687 | 11:105598741 | 58688 | 11:105602391 | 58689 | 11:105602997 |
| 58690 | 11:105604068 | 58691 | 11:105606521 | 58692 | 11:105607752 | 58693 | 11:105608792 | 58694 | 11:105609860 |
| 58695 | 11:105611942 | 58696 | 11:105613558 | 58697 | 11:105615858 | 58698 | 11:105617483 | 58699 | 11:105618287 |
| 58700 | 11:105620854 | 58701 | 11:105621580 | 58702 | 11:105624139 | 58703 | 11:105629294 | 58704 | 11:105670747 |
| 58705 | 11:105692412 | 58707 | 11:105761760 | 58712 | 11:106068659 | 58713 | 11:106083765 | 58715 | 11:106210542 |
| 58717 | 11:106345751 | 58719 | 11:106449495 | 58722 | 11:106574448 | 58723 | 11:106574593 | 58724 | 11:106574854 |
| 58725 | 11:106611790 | 58726 | 11:106614769 | 58727 | 11:106626333 | 58740 | 12:317570 | 58748 | 12:542288 |
| 58757 | 12:877215 | 58797 | 12:1923479 | 58805 | 12:2038461 | 58813 | 12:2256922 | 58819 | 12:2323582 |
| 58825 | 12:2448377 | 58826 | 12:2448435 | 58827 | 12:2452533 | 58828 | 12:2452685 | 58829 | 12:2509817 |
| 58836 | 12:2603168 | 58839 | 12:2644727 | 58840 | 12:2644772 | 58841 | 12:2644793 | 58844 | 12:2731342 |
| 58846 | 12:2771593 | 58923 | 12:4734740 | 58937 | 12:5055337 | 59006 | 12:7477665 | 59040 | 12:8789258 |
| 59041 | 12:8796877 | 59053 | 12:9132925 | 59062 | 12:9382289 | 59066 | 12:9448144 | 59077 | 12:9708609 |
| 59078 | 12:9708626 | 59093 | 12:10135415 | 59095 | 12:10160043 | 59114 | 12:10462955 | 59122 | 12:10621365 |
| 59123 | 12:10622131 | 59124 | 12:10631694 | 59125 | 12:10631700 | 59134 | 12:10781199 | 59152 | 12:11193032 |
| 59170 | 12:11622983 | 59171 | 12:11641143 | 59173 | 12:11670644 | 59175 | 12:11684828 | 59186 | 12:11878651 |
| 59189 | 12:11933919 | 59193 | 12:12026687 | 59195 | 12:12066263 | 59196 | 12:12069687 | 59197 | 12:12087862 |
| 59198 | 12:12123346 | 59206 | 12:12322089 | 59210 | 12:12404824 | 59219 | 12:12760273 | 59224 | 12:12974557 |
| 59231 | 12:13161754 | 59239 | 12:13388932 | 59241 | 12:13411406 | 59242 | 12:13473520 | 59243 | 12:13518818 |
| 59246 | 12:13578439 | 59247 | 12:13591408 | 59250 | 12:13656437 | 59261 | 12:13824057 | 59268 | 12:13927173 |
| 59280 | 12:14131140 | 59281 | 12:14132377 | 59283 | 12:14157283 | 59285 | 12:14211580 | 59286 | 12:14211590 |
| 59287 | 12:14211610 | 59288 | 12:14240924 | 59292 | 12:14361824 | 59293 | 12:14372218 | 59294 | 12:14388145 |
| 59295 | 12:14399933 | 59299 | 12:14456910 | 59302 | 12:14520853 | 59306 | 12:14595181 | 59311 | 12:14716329 |
| 59319 | 12:14988572 | 59321 | 12:15005389 | 59324 | 12:15101552 | 59333 | 12:15266724 | 59335 | 12:15281976 |
| 59344 | 12:15465428 | 59348 | 12:15534714 | 59350 | 12:15552667 | 59352 | 12:15567714 | 59353 | 12:15577383 |
| 59355 | 12:15618825 | 59360 | 12:15729746 | 59363 | 12:15770061 | 59366 | 12:15810335 | 59373 | 12:15938324 |
| 59380 | 12:16125597 | 59381 | 12:16136554 | 59386 | 12:16274543 | 59390 | 12:16346416 | 59394 | 12:16413245 |
| 59410 | 12:16793613 | 59414 | 12:16900417 | 59423 | 12:17162528 | 59425 | 12:17199573 | 59430 | 12:17324217 |
| 59435 | 12:17368614 | 59440 | 12:17470036 | 59453 | 12:17926477 | 59459 | 12:18040185 | 59468 | 12:18229565 |
| 59469 | 12:18253619 | 59474 | 12:18390032 | 59475 | 12:18391314 | 59486 | 12:18697557 | 59492 | 12:18898137 |
| 59496 | 12:18919039 | 59507 | 12:19071459 | 59509 | 12:19100832 | 59517 | 12:19186475 | 59519 | 12:19221055 |
| 59520 | 12:19233240 | 59522 | 12:19351299 | 59523 | 12:19367974 | 59524 | 12:19367977 | 59528 | 12:19460445 |
| 59545 | 12:19931195 | 59550 | 12:20111148 | 59551 | 12:20111169 | 59578 | 12:20706397 | 59582 | 12:20914485 |
| 59588 | 12:21042616 | 59591 | 12:21070776 | 59597 | 12:21212930 | 59600 | 12:21309091 | 59602 | 12:21368915 |
| 59613 | 12:21811288 | 59617 | 12:21924603 | 59618 | 12:21936752 | 59620 | 12:21945571 | 59621 | 12:21969243 |
| 59627 | 12:22146118 | 59631 | 12:22263714 | 59642 | 12:22573355 | 59643 | 12:22576349 | 59647 | 12:22662928 |
| 59653 | 12:22821830 | 59654 | 12:22834458 | 59663 | 12:23060365 | 59667 | 12:23201662 | 59671 | 12:23249052 |
| 59677 | 12:23371031 | 59680 | 12:23439101 | 59696 | 12:23931995 | 59703 | 12:24076421 | 59710 | 12:24256758 |
| 59716 | 12:24331867 | 59721 | 12:24471307 | 59733 | 12:24731505 | 59734 | 12:24748284 | 59737 | 12:24838866 |
| 59741 | 12:24922215 | 59742 | 12:24925957 | 59747 | 12:25022781 | 59749 | 12:25034947 | 59754 | 12:25118490 |
| 59766 | 12:25470724 | 59767 | 12:25498467 | 59769 | 12:25589790 | 59777 | 12:25724323 | 59798 | 12:26442722 |
| 59805 | 12:26653552 | 59808 | 12:26751983 | 59814 | 12:26874891 | 59820 | 12:26979789 | 59821 | 12:26986008 |
| 59830 | 12:27189356 | 59841 | 12:27380634 | 59848 | 12:27503259 | 59850 | 12:27528591 | 59854 | 12:27721180 |
| 59855 | 12:27723132 | 59856 | 12:27723229 | 59861 | 12:27998192 | 59862 | 12:28002030 | 59878 | 12:28526148 |
| 59879 | 12:28587383 | 59881 | 12:28621183 | 59893 | 12:28989343 | 59913 | 12:29539899 | 59934 | 12:30141838 |
| 59944 | 12:30381579 | 59945 | 12:30411312 | 59947 | 12:30444618 | 59949 | 12:30463422 | 59969 | 12:30909596 |
| 59971 | 12:30979325 | 59973 | 12:30983862 | 59974 | 12:30991739 | 59977 | 12:31053899 | 59979 | 12:31059835 |
| 59980 | 12:31115741 | 59984 | 12:31247300 | 59985 | 12:31247421 | 59986 | 12:31247454 | 59987 | 12:31247486 |
| 59998 | 12:31454833 | 60007 | 12:31767190 | 60011 | 12:31790778 | 60012 | 12:31797116 | 60018 | 12:31950273 |
| 60023 | 12:32162560 | 60028 | 12:32295933 | 60029 | 12:32338936 | 60032 | 12:32379592 | 60033 | 12:32387590 |
| 60034 | 12:32391405 | 60037 | 12:32450230 | 60038 | 12:32450232 | 60040 | 12:32487563 | 60058 | 12:33054434 |
| 60061 | 12:33082346 | 60062 | 12:33085573 | 60066 | 12:33139330 | 60075 | 12:33340233 | 60086 | 12:33674224 |
| 60095 | 12:33970418 | 60096 | 12:34054782 | 60097 | 12:34068458 | 60109 | 12:34500254 | 60114 | 12:34681917 |
| 60126 | 12:34900938 | 60133 | 12:35115665 | 60140 | 12:35310855 | 60150 | 12:35346851 | 60164 | 12:35747252 |
| 60173 | 12:36216803 | 60175 | 12:36341507 | 60176 | 12:36361684 | 60178 | 12:36421378 | 60179 | 12:36443014 |
| 60181 | 12:36474506 | 60182 | 12:36483162 | 60183 | 12:36486137 | 60184 | 12:36504213 | 60189 | 12:36706435 |
| 60191 | 12:36753159 | 60195 | 12:36952382 | 60196 | 12:36952416 | 60210 | 12:37329971 | 60211 | 12:37330062 |
| 60212 | 12:37330099 | 60216 | 12:37420551 | 60256 | 12:39391630 | 60260 | 12:39553608 | 60267 | 12:39684127 |
| 60270 | 12:39776454 | 60279 | 12:40049870 | 60281 | 12:40141911 | 60311 | 12:41076939 | 60329 | 12:41861946 |
| 60332 | 12:41912763 | 60402 | 12:44937336 | 60404 | 12:44979318 | 60422 | 12:45617161 | 60442 | 12:46329415 |
| 60445 | 12:46430991 | 60466 | 12:46961986 | 60470 | 12:47018801 | 60474 | 12:47110627 | 60484 | 12:47309976 |
| 60487 | 12:47407515 | 60508 | 12:48047961 | 60510 | 12:48150915 | 60538 | 12:48960299 | 60549 | 12:49199679 |
| 60552 | 12:49236831 | 60566 | 12:49561422 | 60571 | 12:49723456 | 60574 | 12:49789492 | 60579 | 12:49873319 |
| 60580 | 12:49881472 | 60584 | 12:49919596 | 60600 | 12:50189626 | 60609 | 12:50384404 | 60611 | 12:50424103 |
| 60612 | 12:50439814 | 60614 | 12:50497707 | 60615 | 12:50517145 | 60616 | 12:50525554 | 60617 | 12:50530876 |
| 60619 | 12:50579721 | 60628 | 12:50806532 | 60630 | 12:50840958 | 60632 | 12:50869818 | 60633 | 12:50871829 |
| 60660 | 12:51551817 | 60661 | 12:51626384 | 60679 | 12:52103992 | 60687 | 12:52262646 | 60692 | 12:52346386 |
| 60702 | 12:52660295 | 60710 | 12:52806268 | 60727 | 12:53053258 | 60729 | 12:53061165 | 60738 | 12:53338165 |
| 60739 | 12:53339046 | 60740 | 12:53339054 | 60749 | 12:53523285 | 60750 | 12:53553256 | 60752 | 12:53587669 |
| 60755 | 12:53627763 | 60756 | 12:53640047 | 60763 | 12:53801258 | 60781 | 12:54131348 | 60782 | 12:54153871 |
| 60797 | 12:54494995 | 60803 | 12:54630943 | 60804 | 12:54634286 | 60810 | 12:54736016 | 60813 | 12:54833257 |
| 60815 | 12:54927343 | 60819 | 12:55024877 | 60821 | 12:55068443 | 60848 | 12:55943119 | 60851 | 12:56039366 |
| 60880 | 12:56976868 | 60882 | 12:57050432 | 60904 | 12:57798387 | 60905 | 12:57798425 | 60912 | 12:57952881 |
| 60914 | 12:58028126 | 60929 | 12:58672694 | 60957 | 12:59417372 | 60969 | 12:59853749 | 60978 | 12:60236147 |
| 60990 | 12:60736444 | 61018 | 12:61855777 | 61024 | 12:62071314 | 61028 | 12:62095959 | 61031 | 12:62215048 |
| 61034 | 12:62291744 | 61035 | 12:62297558 | 61051 | 12:62665927 | 61053 | 12:62697756 | 61065 | 12:63053238 |
| 61080 | 12:63589109 | 61085 | 12:63730818 | 61089 | 12:63774437 | 61094 | 12:63920843 | 61100 | 12:64086485 |
| 61112 | 12:64454937 | 61126 | 12:64874897 | 61127 | 12:64896393 | 61148 | 12:65376279 | 61151 | 12:65433100 |
| 61156 | 12:65616303 | 61168 | 12:66123917 | 61170 | 12:66204351 | 61175 | 12:66340744 | 61199 | 12:66896772 |
| 61206 | 12:67184352 | 61207 | 12:67228835 | 61210 | 12:67289617 | 61216 | 12:67417050 | 61231 | 12:67757020 |
| 61237 | 12:67889485 | 61252 | 12:68144427 | 61255 | 12:68171444 | 61256 | 12:68171491 | 61262 | 12:68316664 |
| 61274 | 12:68605490 | 61280 | 12:68760089 | 61283 | 12:68784416 | 61289 | 12:68861830 | 61321 | 12:69512279 |
| 61322 | 12:69512294 | 61323 | 12:69516800 | 61331 | 12:69577020 | 61338 | 12:69681798 | 61348 | 12:70147062 |
| 61367 | 12:71314524 | 61383 | 12:71639973 | 61386 | 12:71790421 | 61390 | 12:71842435 | 61408 | 12:72931638 |
| 61414 | 12:73012734 | 61421 | 12:73132371 | 61422 | 12:73154971 | 61427 | 12:73317927 | 61428 | 12:73324264 |
| 61441 | 12:73504167 | 61443 | 12:73515714 | 61444 | 12:73515717 | 61445 | 12:73515812 | 61454 | 12:73622472 |
| 61455 | 12:73637550 | 61462 | 12:73750586 | 61469 | 12:73899680 | 61475 | 12:74054821 | 61486 | 12:74328198 |
| 61495 | 12:74492364 | 61498 | 12:74505038 | 61500 | 12:74526665 | 61517 | 12:74949213 | 61530 | 12:75243236 |
| 61535 | 12:75318404 | 61538 | 12:75375959 | 61542 | 12:75403639 | 61543 | 12:75471804 | 61558 | 12:75839630 |
| 61567 | 12:76074190 | 61569 | 12:76110122 | 61581 | 12:76477335 | 61587 | 12:76644169 | 61588 | 12:76649683 |
| 61605 | 12:77139238 | 61609 | 12:77240177 | 61627 | 12:77604409 | 61632 | 12:77695108 | 61641 | 12:77891817 |
| 61644 | 12:77992115 | 61647 | 12:78041886 | 61655 | 12:78246588 | 61669 | 12:78577349 | 61691 | 12:79095439 |
| 61692 | 12:79098661 | 61693 | 12:79098791 | 61694 | 12:79099207 | 61695 | 12:79099246 | 61696 | 12:79104872 |
| 61710 | 12:79403358 | 61715 | 12:79476337 | 61727 | 12:79736085 | 61740 | 12:79970178 | 61756 | 12:80319360 |
| 61775 | 12:80804698 | 61790 | 12:81214960 | 61791 | 12:81215027 | 61803 | 12:81507963 | 61804 | 12:81508005 |
| 61819 | 12:81781296 | 61820 | 12:81840070 | 61824 | 12:81902349 | 61840 | 12:82263825 | 61845 | 12:82445861 |
| 61848 | 12:82510342 | 61877 | 12:83071777 | 61882 | 12:83242885 | 61891 | 12:83362092 | 61907 | 12:83699595 |
| 61915 | 12:83925298 | 61920 | 12:84006533 | 61926 | 12:84137136 | 61945 | 12:84691330 | 61946 | 12:84709160 |
| 61948 | 12:84744945 | 61949 | 12:84744950 | 61955 | 12:84883821 | 61960 | 12:84929565 | 61966 | 12:84993056 |
| 61969 | 12:85038979 | 61970 | 12:85051267 | 61973 | 12:85122781 | 61978 | 12:85226982 | 61979 | 12:85230036 |
| 61981 | 12:85245683 | 61983 | 12:85268459 | 61984 | 12:85293670 | 61986 | 12:85321955 | 61988 | 12:85328690 |
| 61990 | 12:85352315 | 61991 | 12:85354082 | 61994 | 12:85362756 | 61995 | 12:85375388 | 61997 | 12:85396298 |
| 62000 | 12:85462507 | 62007 | 12:85632578 | 62013 | 12:85790348 | 62014 | 12:85790349 | 62035 | 12:86313847 |
| 62038 | 12:86341413 | 62043 | 12:86507763 | 62045 | 12:86516372 | 62046 | 12:86516421 | 62048 | 12:86519761 |
| 62049 | 12:86565634 | 62051 | 12:86656646 | 62052 | 12:86687289 | 62053 | 12:86688052 | 62056 | 12:86734179 |
| 62058 | 12:86744155 | 62059 | 12:86764063 | 62062 | 12:86796745 | 62064 | 12:86824616 | 62068 | 12:86895391 |
| 62069 | 12:86895562 | 62070 | 12:86897090 | 62071 | 12:86897178 | 62072 | 12:86901814 | 62073 | 12:86901834 |
| 62074 | 12:86901846 | 62075 | 12:86901945 | 62076 | 12:86902939 | 62077 | 12:86902946 | 62078 | 12:86907527 |
| 62079 | 12:86907549 | 62080 | 12:86908054 | 62082 | 12:86924401 | 62085 | 12:86976449 | 62086 | 12:86988484 |
| 62089 | 12:87019731 | 62090 | 12:87024506 | 62095 | 12:87094553 | 62109 | 13:837989 | 62110 | 13:859954 |
| 62115 | 13:877219 | 62122 | 13:1025761 | 62126 | 13:1174223 | 62127 | 13:1184941 | 62138 | 13:1435091 |
| 62143 | 13:1589719 | 62145 | 13:1615667 | 62154 | 13:1741335 | 62155 | 13:1754933 | 62165 | 13:2014401 |
| 62169 | 13:2079335 | 62174 | 13:2108318 | 62176 | 13:2133989 | 62181 | 13:2232512 | 62188 | 13:2272732 |
| 62193 | 13:2391867 | 62194 | 13:2394109 | 62195 | 13:2400920 | 62196 | 13:2402378 | 62200 | 13:2507817 |
| 62203 | 13:2551762 | 62208 | 13:2625076 | 62209 | 13:2632788 | 62238 | 13:3279851 | 62250 | 13:3588425 |
| 62254 | 13:3701619 | 62259 | 13:3860445 | 62265 | 13:3974443 | 62270 | 13:4084139 | 62271 | 13:4084171 |
| 62272 | 13:4084181 | 62276 | 13:4221428 | 62278 | 13:4302438 | 62291 | 13:4792202 | 62292 | 13:4793338 |
| 62312 | 13:5324308 | 62314 | 13:5360261 | 62325 | 13:5525194 | 62326 | 13:5525225 | 62327 | 13:5525250 |
| 62341 | 13:5742073 | 62356 | 13:6112364 | 62377 | 13:6686807 | 62379 | 13:6694933 | 62384 | 13:6837817 |
| 62392 | 13:7036389 | 62399 | 13:7224958 | 62406 | 13:7441639 | 62412 | 13:7553513 | 62425 | 13:7848557 |
| 62441 | 13:8487756 | 62465 | 13:8980654 | 62477 | 13:9357735 | 62486 | 13:9550333 | 62487 | 13:9576478 |
| 62496 | 13:9729764 | 62497 | 13:9729772 | 62512 | 13:10026414 | 62514 | 13:10070381 | 62522 | 13:10306871 |
| 62523 | 13:10395756 | 62529 | 13:10478767 | 62533 | 13:10517565 | 62542 | 13:10720404 | 62543 | 13:10724228 |
| 62547 | 13:10815809 | 62549 | 13:10956279 | 62550 | 13:10956346 | 62551 | 13:10956354 | 62552 | 13:10956355 |
| 62559 | 13:11536433 | 62561 | 13:11561251 | 62562 | 13:11598409 | 62566 | 13:11792199 | 62568 | 13:11904569 |
| 62581 | 13:12520500 | 62584 | 13:12616676 | 62585 | 13:12669714 | 62593 | 13:12825549 | 62597 | 13:12901657 |
| 62601 | 13:12957260 | 62604 | 13:13014675 | 62606 | 13:13050532 | 62609 | 13:13134686 | 62620 | 13:13394218 |
| 62624 | 13:13416442 | 62629 | 13:13525172 | 62633 | 13:13724404 | 62648 | 13:14180541 | 62709 | 13:15922152 |
| 62710 | 13:15926948 | 62713 | 13:16113457 | 62714 | 13:16141551 | 62715 | 13:16161619 | 62716 | 13:16198035 |
| 62727 | 13:16479721 | 62752 | 13:16821257 | 62753 | 13:16838893 | 62758 | 13:16979210 | 62766 | 13:17176766 |
| 62775 | 13:17311025 | 62776 | 13:17311026 | 62777 | 13:17311048 | 62778 | 13:17311402 | 62779 | 13:17313556 |
| 62780 | 13:17321384 | 62781 | 13:17327212 | 62784 | 13:17371815 | 62791 | 13:17532109 | 62792 | 13:17537709 |
| 62793 | 13:17537777 | 62794 | 13:17537795 | 62795 | 13:17540260 | 62796 | 13:17540346 | 62797 | 13:17543085 |
| 62800 | 13:17568204 | 62804 | 13:17707891 | 62805 | 13:17739721 | 62807 | 13:17780066 | 62809 | 13:17793717 |
| 62810 | 13:17796108 | 62811 | 13:17797006 | 62812 | 13:17797028 | 62813 | 13:17797078 | 62814 | 13:17800892 |
| 62819 | 13:17959881 | 62821 | 13:18000186 | 62822 | 13:18000196 | 62823 | 13:18001014 | 62824 | 13:18001496 |
| 62825 | 13:18001505 | 62826 | 13:18001601 | 62828 | 13:18117994 | 62831 | 13:18160335 | 62843 | 13:18626070 |
| 62847 | 13:18666175 | 62853 | 13:18721208 | 62881 | 13:18755905 | 62883 | 13:18792716 | 62884 | 13:18792825 |
| 62885 | 13:18792850 | 62889 | 13:18828811 | 62891 | 13:18892498 | 62892 | 13:18897253 | 62894 | 13:19005851 |
| 62895 | 13:19009052 | 62896 | 13:19009093 | 62897 | 13:19009113 | 62902 | 13:19106619 | 62911 | 13:19331702 |
| 62917 | 13:19457766 | 62919 | 13:19497053 | 62920 | 13:19497075 | 62921 | 13:19497132 | 62922 | 13:19497137 |
| 62923 | 13:19539755 | 62924 | 13:19567930 | 62926 | 13:19588565 | 62929 | 13:19655690 | 62930 | 13:19688739 |
| 62933 | 13:19726581 | 62935 | 13:19733380 | 62941 | 13:19822361 | 62942 | 13:19829540 | 62948 | 13:19908550 |
| 62950 | 13:19929408 | 62951 | 13:19985595 | 62966 | 13:20376052 | 62973 | 13:20455853 | 62974 | 13:20455858 |
| 62975 | 13:20455892 | 62976 | 13:20455934 | 62977 | 13:20455941 | 62986 | 13:20690765 | 62989 | 13:20711502 |
| 63003 | 13:21079068 | 63004 | 13:21080622 | 63005 | 13:21080632 | 63006 | 13:21080639 | 63007 | 13:21080653 |
| 63008 | 13:21080676 | 63009 | 13:21080695 | 63015 | 13:21196496 | 63021 | 13:21338503 | 63025 | 13:21463530 |
| 63033 | 13:21602178 | 63034 | 13:21608876 | 63036 | 13:21617194 | 63037 | 13:21669388 | 63038 | 13:21673325 |
| 63046 | 13:21871770 | 63053 | 13:22005224 | 63067 | 13:22364650 | 63069 | 13:22400352 | 63075 | 13:22556435 |
| 63083 | 13:22843425 | 63085 | 13:22943752 | 63094 | 13:23364320 | 63095 | 13:23428241 | 63100 | 13:23590170 |
| 63107 | 13:23799970 | 63113 | 13:23993051 | 63122 | 13:24204881 | 63125 | 13:24378395 | 63128 | 13:24432283 |
| 63129 | 13:24464855 | 63138 | 13:24550804 | 63144 | 13:24721448 | 63148 | 13:24785943 | 63152 | 13:24889747 |
| 63157 | 13:25026767 | 63162 | 13:25139101 | 63163 | 13:25157343 | 63164 | 13:25169602 | 63171 | 13:25314039 |
| 63176 | 13:25373782 | 63177 | 13:25408450 | 63178 | 13:25410196 | 63185 | 13:25553780 | 63186 | 13:25556386 |
| 63197 | 13:25892715 | 63200 | 13:25911303 | 63201 | 13:25911339 | 63202 | 13:25911366 | 63204 | 13:25958875 |
| 63207 | 13:26100997 | 63216 | 13:26381171 | 63217 | 13:26385197 | 63218 | 13:26403532 | 63228 | 13:26709761 |
| 63229 | 13:26798911 | 63246 | 13:27211452 | 63249 | 13:27252426 | 63255 | 13:27371552 | 63259 | 13:27465175 |
| 63260 | 13:27465215 | 63261 | 13:27465241 | 63262 | 13:27465291 | 63264 | 13:27517101 | 63267 | 13:27585269 |
| 63269 | 13:27634615 | 63274 | 13:27760115 | 63275 | 13:27831742 | 63276 | 13:27837915 | 63277 | 13:27868901 |
| 63278 | 13:27871914 | 63279 | 13:27897181 | 63280 | 13:27902374 | 63281 | 13:27904185 | 63293 | 13:28285297 |
| 63298 | 13:28416787 | 63306 | 13:28593403 | 63307 | 13:28631602 | 63309 | 13:28668818 | 63317 | 13:28768035 |
| 63322 | 13:28950265 | 63330 | 13:29182643 | 63337 | 13:29330098 | 63339 | 13:29336139 | 63350 | 13:29535530 |
| 63353 | 13:29588319 | 63357 | 13:29751352 | 63369 | 13:30024809 | 63371 | 13:30056042 | 63375 | 13:30104514 |
| 63376 | 13:30183328 | 63377 | 13:30183348 | 63378 | 13:30194908 | 63379 | 13:30194914 | 63380 | 13:30194921 |
| 63381 | 13:30194933 | 63382 | 13:30195026 | 63383 | 13:30195496 | 63385 | 13:30210577 | 63388 | 13:30242235 |
| 63408 | 13:30707478 | 63410 | 13:30754086 | 63418 | 13:30976341 | 63422 | 13:31020731 | 63437 | 13:31276749 |
| 63438 | 13:31276801 | 63440 | 13:31276837 | 63441 | 13:31280242 | 63442 | 13:31280491 | 63443 | 13:31282636 |
| 63444 | 13:31282742 | 63445 | 13:31282787 | 63446 | 13:31285967 | 63447 | 13:31285968 | 63448 | 13:31285972 |
| 63449 | 13:31286001 | 63450 | 13:31286035 | 63451 | 13:31296423 | 63456 | 13:31420376 | 63459 | 13:31447543 |
| 63466 | 13:31582342 | 63471 | 13:31736295 | 63488 | 13:32202239 | 63491 | 13:32240332 | 63496 | 13:32324178 |
| 63497 | 13:32362115 | 63499 | 13:32365033 | 63527 | 13:32808897 | 63538 | 13:33049577 | 63540 | 13:33078146 |
| 63545 | 13:33129981 | 63547 | 13:33172919 | 63549 | 13:33261503 | 63554 | 13:33326303 | 63561 | 13:33610056 |
| 63562 | 13:33610145 | 63563 | 13:33615532 | 63564 | 13:33641897 | 63566 | 13:33682852 | 63569 | 13:33817581 |
| 63570 | 13:33818040 | 63573 | 13:33900260 | 63584 | 13:34538815 | 63586 | 13:34570424 | 63587 | 13:34574425 |
| 63588 | 13:34595352 | 63592 | 13:34690679 | 63594 | 13:34725177 | 63596 | 13:34746582 | 63597 | 13:34750823 |
| 63598 | 13:34798730 | 63603 | 13:34909612 | 63607 | 13:35010608 | 63608 | 13:35019310 | 63610 | 13:35093002 |
| 63613 | 13:35138794 | 63616 | 13:35156141 | 63620 | 13:35193780 | 63624 | 13:35229937 | 63626 | 13:35230259 |
| 63629 | 13:35259809 | 63630 | 13:35273048 | 63632 | 13:35319711 | 63674 | 13:36409030 | 63675 | 13:36424590 |
| 63680 | 13:36570662 | 63682 | 13:36589718 | 63687 | 13:36656277 | 63691 | 13:36716029 | 63694 | 13:36827015 |
| 63696 | 13:36854156 | 63720 | 13:37443281 | 63732 | 13:37745409 | 63742 | 13:37991091 | 63757 | 13:38329758 |
| 63758 | 13:38375463 | 63760 | 13:38420421 | 63762 | 13:38501340 | 63776 | 13:38828149 | 63777 | 13:38828176 |
| 63778 | 13:38828210 | 63779 | 13:38828306 | 63780 | 13:38828333 | 63784 | 13:38935644 | 63787 | 13:38957716 |
| 63806 | 13:39270018 | 63808 | 13:39276440 | 63813 | 13:39359409 | 63814 | 13:39360537 | 63817 | 13:39403535 |
| 63821 | 13:39464756 | 63822 | 13:39498069 | 63824 | 13:39559729 | 63825 | 13:39574123 | 63826 | 13:39584391 |
| 63828 | 13:39644803 | 63834 | 13:39704818 | 63847 | 13:40027121 | 63901 | 13:41141472 | 63926 | 13:41826984 |
| 63928 | 13:41862833 | 63929 | 13:41863502 | 63944 | 13:42197339 | 63952 | 13:42439893 | 63956 | 13:42526261 |
| 63961 | 13:42557806 | 63963 | 13:42567447 | 63964 | 13:42570926 | 63965 | 13:42576337 | 63966 | 13:42578756 |
| 63967 | 13:42579747 | 63968 | 13:42581051 | 63969 | 13:42583033 | 63970 | 13:42585954 | 63971 | 13:42587350 |
| 63973 | 13:42588548 | 63974 | 13:42589734 | 63975 | 13:42590562 | 63976 | 13:42591034 | 63977 | 13:42591591 |
| 63978 | 13:42592688 | 63979 | 13:42602259 | 63980 | 13:42604226 | 63981 | 13:42605060 | 63982 | 13:42606861 |
| 63983 | 13:42607539 | 63984 | 13:42617225 | 63985 | 13:42619772 | 63986 | 13:42620639 | 63987 | 13:42622791 |
| 63988 | 13:42624718 | 63990 | 13:42628160 | 63991 | 13:42632412 | 63992 | 13:42636254 | 63994 | 13:42655005 |
| 63995 | 13:42655747 | 63997 | 13:42707046 | 63998 | 13:42733395 | 64000 | 13:42775404 | 64002 | 13:42816216 |
| 64010 | 13:43060399 | 64013 | 13:43174367 | 64017 | 13:43269543 | 64018 | 13:43342598 | 64019 | 13:43400200 |
| 64021 | 13:43503495 | 64030 | 13:43617243 | 64034 | 13:43735619 | 64035 | 13:43736152 | 64061 | 13:44136436 |
| 64064 | 13:44159827 | 64066 | 13:44231058 | 64070 | 13:44319398 | 64073 | 13:44364216 | 64079 | 13:44475918 |
| 64080 | 13:44491717 | 64091 | 13:44736057 | 64116 | 13:44978580 | 64117 | 13:44978611 | 64119 | 13:44989622 |
| 64128 | 13:45018113 | 64129 | 13:45022424 | 64130 | 13:45034606 | 64132 | 13:45038863 | 64133 | 13:45046560 |
| 64135 | 13:45057233 | 64136 | 13:45081132 | 64138 | 13:45099582 | 64139 | 13:45108541 | 64140 | 13:45110718 |
| 64141 | 13:45112674 | 64142 | 13:45114484 | 64144 | 13:45129342 | 64145 | 13:45135756 | 64146 | 13:45143400 |
| 64147 | 13:45144409 | 64149 | 13:45160765 | 64173 | 13:45837065 | 64188 | 13:46248201 | 64189 | 13:46260736 |
| 64190 | 13:46262101 | 64191 | 13:46280773 | 64192 | 13:46303988 | 64193 | 13:46308862 | 64197 | 13:46342659 |
| 64203 | 13:46433525 | 64205 | 13:46541983 | 64217 | 13:46759585 | 64219 | 13:46767424 | 64222 | 13:46855501 |
| 64226 | 13:46934942 | 64230 | 13:47057469 | 64235 | 13:47187424 | 64240 | 13:47305456 | 64242 | 13:47333121 |
| 64243 | 13:47346135 | 64265 | 13:47860032 | 64267 | 13:47916526 | 64268 | 13:47927079 | 64270 | 13:47952062 |
| 64271 | 13:48003258 | 64272 | 13:48017728 | 64279 | 13:48210178 | 64281 | 13:48212858 | 64313 | 13:48933264 |
| 64327 | 13:49369652 | 64371 | 13:50683553 | 64377 | 13:50955038 | 64379 | 13:50985036 | 64385 | 13:51191490 |
| 64388 | 13:51268713 | 64389 | 13:51269069 | 64404 | 13:51645253 | 64408 | 13:51745594 | 64412 | 13:51889992 |
| 64413 | 13:51917960 | 64424 | 13:52280855 | 64425 | 13:52333810 | 64430 | 13:52514427 | 64436 | 13:52666460 |
| 64443 | 13:52794690 | 64444 | 13:52797973 | 64445 | 13:52798735 | 64446 | 13:52798786 | 64448 | 13:52806170 |
| 64450 | 13:52838262 | 64457 | 13:53001236 | 64461 | 13:53139865 | 64463 | 13:53146985 | 64465 | 13:53149275 |
| 64468 | 13:53184275 | 64470 | 13:53189308 | 64471 | 13:53191327 | 64472 | 13:53192744 | 64473 | 13:53193885 |
| 64474 | 13:53195323 | 64475 | 13:53197934 | 64476 | 13:53199665 | 64477 | 13:53200513 | 64478 | 13:53201494 |
| 64479 | 13:53205894 | 64480 | 13:53211390 | 64481 | 13:53212642 | 64482 | 13:53213246 | 64483 | 13:53214256 |
| 64484 | 13:53221957 | 64485 | 13:53223771 | 64486 | 13:53225335 | 64490 | 13:53267383 | 64491 | 13:53287399 |
| 64502 | 13:53656779 | 64503 | 13:53668817 | 64521 | 13:54501975 | 64524 | 13:54544791 | 64528 | 13:54637805 |
| 64532 | 13:54688808 | 64539 | 13:54867617 | 64540 | 13:54924099 | 64541 | 13:54930460 | 64542 | 13:54963877 |
| 64545 | 13:55001775 | 64546 | 13:55009029 | 64549 | 13:55107919 | 64551 | 13:55137013 | 64553 | 13:55164494 |
| 64556 | 13:55256797 | 64558 | 13:55286529 | 64559 | 13:55306487 | 64563 | 13:55363256 | 64576 | 13:55652395 |
| 64579 | 13:55736298 | 64586 | 13:55924074 | 64596 | 13:56129806 | 64599 | 13:56206409 | 64602 | 13:56257733 |
| 64613 | 13:56553088 | 64615 | 13:56567143 | 64616 | 13:56574066 | 64628 | 13:56987530 | 64632 | 13:57050196 |
| 64642 | 13:57391199 | 64643 | 13:57394025 | 64654 | 13:57698221 | 64655 | 13:57736295 | 64656 | 13:57744564 |
| 64657 | 13:57744624 | 64664 | 13:57962862 | 64677 | 13:58287555 | 64678 | 13:58288391 | 64679 | 13:58297881 |
| 64683 | 13:58337666 | 64688 | 13:58408741 | 64689 | 13:58453137 | 64690 | 13:58475365 | 64693 | 13:58521548 |
| 64700 | 13:58687733 | 64703 | 13:58755844 | 64705 | 13:58823450 | 64709 | 13:58925642 | 64711 | 13:58935983 |
| 64713 | 13:58968219 | 64718 | 13:58981092 | 64737 | 13:59317532 | 64738 | 13:59317573 | 64739 | 13:59317600 |
| 64752 | 13:59798919 | 64753 | 13:59810522 | 64757 | 13:59881900 | 64763 | 13:59982505 | 64765 | 13:60004397 |
| 64775 | 13:60191346 | 64777 | 13:60219559 | 64779 | 13:60249957 | 64781 | 13:60270738 | 64782 | 13:60273459 |
| 64786 | 13:60360621 | 64787 | 13:60363893 | 64803 | 13:60828808 | 64813 | 13:61235720 | 64819 | 13:61487750 |
| 64823 | 13:61526661 | 64824 | 13:61536675 | 64830 | 13:61748180 | 64833 | 13:61837125 | 64857 | 13:62340129 |
| 64864 | 13:62437505 | 64865 | 13:62437664 | 64869 | 13:62616409 | 64870 | 13:62619495 | 64871 | 13:62620895 |
| 64872 | 13:62620917 | 64873 | 13:62623230 | 64874 | 13:62623246 | 64875 | 13:62623260 | 64876 | 13:62623271 |
| 64877 | 13:62623277 | 64878 | 13:62623296 | 64879 | 13:62623326 | 64890 | 13:62758151 | 64895 | 13:62805225 |
| 64896 | 13:62808252 | 64898 | 13:62825728 | 64903 | 13:62901932 | 64904 | 13:62913802 | 64905 | 13:62957243 |
| 64909 | 13:63100717 | 64910 | 13:63113668 | 64911 | 13:63115815 | 64912 | 13:63117671 | 64915 | 13:63231676 |
| 64916 | 13:63238472 | 64922 | 13:63381167 | 64924 | 13:63406383 | 64927 | 13:63465271 | 64932 | 13:63632674 |
| 64933 | 13:63667387 | 64936 | 13:63800029 | 64941 | 13:63926705 | 64944 | 13:63945489 | 64945 | 13:63950962 |
| 64964 | 13:64427414 | 64970 | 13:64579641 | 64973 | 13:64608490 | 64979 | 13:64765574 | 64981 | 13:64780081 |
| 64985 | 13:64917447 | 64992 | 13:65197131 | 64993 | 13:65222405 | 64994 | 13:65226257 | 64996 | 13:65300967 |
| 65002 | 13:65421703 | 65003 | 13:65421742 | 65008 | 13:65551485 | 65009 | 13:65556446 | 65012 | 13:65615123 |
| 65013 | 13:65633719 | 65015 | 13:65678606 | 65016 | 13:65710639 | 65017 | 13:65711755 | 65018 | 13:65713224 |
| 65019 | 13:65713273 | 65020 | 13:65714024 | 65022 | 13:65714678 | 65024 | 13:65734511 | 65025 | 13:65737670 |
| 65026 | 13:65747364 | 65027 | 13:65751601 | 65028 | 13:65757675 | 65029 | 13:65768152 | 65030 | 13:65769345 |
| 65031 | 13:65771689 | 65032 | 13:65778554 | 65038 | 13:65891788 | 65045 | 13:66156547 | 65051 | 13:66242964 |
| 65054 | 13:66269753 | 65058 | 13:66340843 | 65068 | 13:66611398 | 65078 | 13:66830214 | 65084 | 13:66995569 |
| 65087 | 13:67120106 | 65103 | 13:67448820 | 65126 | 13:68187572 | 65134 | 13:68308181 | 65136 | 13:68378513 |
| 65138 | 13:68519206 | 65140 | 13:68528941 | 65142 | 13:68635086 | 65146 | 13:68672438 | 65152 | 13:68880703 |
| 65165 | 13:69217542 | 65168 | 13:69272129 | 65170 | 13:69348741 | 65171 | 13:69353425 | 65173 | 13:69380590 |
| 65183 | 13:69728600 | 65184 | 13:69731770 | 65187 | 13:69850661 | 65189 | 13:69882420 | 65190 | 13:69890851 |
| 65198 | 13:70040165 | 65201 | 13:70082692 | 65207 | 13:70199117 | 65220 | 13:70568959 | 65225 | 13:70651503 |
| 65227 | 13:70673685 | 65248 | 13:71190284 | 65267 | 13:71537155 | 65272 | 13:71649810 | 65276 | 13:71778746 |
| 65278 | 13:71794746 | 65289 | 13:72099141 | 65290 | 13:72099160 | 65293 | 13:72190824 | 65298 | 13:72223856 |
| 65299 | 13:72240445 | 65300 | 13:72247438 | 65308 | 13:72487250 | 65311 | 13:72581815 | 65316 | 13:72835577 |
| 65318 | 13:72837581 | 65319 | 13:72837712 | 65320 | 13:72837736 | 65323 | 13:72873627 | 65326 | 13:72911160 |
| 65327 | 13:72931732 | 65331 | 13:72989401 | 65333 | 13:73030191 | 65342 | 13:73241000 | 65353 | 13:73412480 |
| 65358 | 13:73475168 | 65359 | 13:73486255 | 65360 | 13:73508637 | 65361 | 13:73516499 | 65362 | 13:73552702 |
| 65363 | 13:73554377 | 65367 | 13:73602251 | 65369 | 13:73628006 | 65370 | 13:73628032 | 65372 | 13:73657151 |
| 65373 | 13:73659243 | 65375 | 13:73684336 | 65376 | 13:73720189 | 65377 | 13:73738431 | 65378 | 13:73747745 |
| 65386 | 13:74002990 | 65388 | 13:74075881 | 65396 | 13:74272611 | 65397 | 13:74284172 | 65405 | 13:74425124 |
| 65406 | 13:74461134 | 65407 | 13:74464989 | 65408 | 13:74467020 | 65409 | 13:74470296 | 65410 | 13:74476473 |
| 65411 | 13:74479132 | 65412 | 13:74479161 | 65417 | 13:74575589 | 65419 | 13:74593675 | 65421 | 13:74635628 |
| 65423 | 13:74640625 | 65426 | 13:74665130 | 65427 | 13:74678375 | 65432 | 13:74760409 | 65433 | 13:74815833 |
| 65437 | 13:74932791 | 65455 | 13:75521831 | 65463 | 13:75584984 | 65464 | 13:75584987 | 65465 | 13:75585044 |
| 65473 | 13:75757004 | 65474 | 13:75767512 | 65478 | 13:75797537 | 65479 | 13:75808921 | 65481 | 13:75817367 |
| 65496 | 13:76209741 | 65503 | 13:76376258 | 65506 | 13:76438473 | 65507 | 13:76438492 | 65508 | 13:76442151 |
| 65509 | 13:76443128 | 65521 | 13:76672357 | 65523 | 13:76717525 | 65527 | 13:76772665 | 65534 | 13:76884649 |
| 65545 | 13:77225860 | 65551 | 13:77281427 | 65555 | 13:77332710 | 65557 | 13:77387866 | 65560 | 13:77423447 |
| 65561 | 13:77443501 | 65572 | 13:77715514 | 65574 | 13:77719500 | 65576 | 13:77810786 | 65579 | 13:77851241 |
| 65580 | 13:77856986 | 65584 | 13:78019495 | 65589 | 13:78097466 | 65590 | 13:78097543 | 65591 | 13:78097572 |
| 65595 | 13:78246967 | 65600 | 13:78323978 | 65601 | 13:78327563 | 65611 | 13:78606738 | 65620 | 13:78732308 |
| 65628 | 13:78862443 | 65630 | 13:78895292 | 65636 | 13:79010034 | 65646 | 13:79138574 | 65653 | 13:79289518 |
| 65663 | 13:79372632 | 65664 | 13:79378945 | 65670 | 13:79439113 | 65746 | 13:80685975 | 65753 | 13:80868488 |
| 65754 | 13:80869352 | 65775 | 13:81361992 | 65780 | 13:81441602 | 65789 | 13:81578319 | 65793 | 13:81659411 |
| 65796 | 13:81695713 | 65809 | 13:81975437 | 65811 | 13:82042702 | 65857 | 13:82944108 | 65859 | 13:82944150 |
| 65870 | 13:83194337 | 65885 | 14:260388 | 65890 | 14:344042 | 65892 | 14:428398 | 65906 | 14:1024267 |
| 65907 | 14:1025055 | 65908 | 14:1033297 | 65909 | 14:1036134 | 65910 | 14:1039518 | 65911 | 14:1044777 |
| 65914 | 14:1153408 | 65916 | 14:1188418 | 65919 | 14:1314992 | 65923 | 14:1388615 | 65924 | 14:1394179 |
| 65928 | 14:1554543 | 65930 | 14:1658546 | 65931 | 14:1670892 | 65932 | 14:1671183 | 65933 | 14:1671573 |
| 65934 | 14:1671580 | 65935 | 14:1681783 | 65936 | 14:1702340 | 65940 | 14:1785051 | 65941 | 14:1798788 |
| 65942 | 14:1799618 | 65943 | 14:1799673 | 65944 | 14:1800049 | 65946 | 14:1844464 | 65947 | 14:1854457 |
| 65950 | 14:1926589 | 65951 | 14:1946346 | 65966 | 14:2415920 | 65968 | 14:2500509 | 65973 | 14:2669589 |
| 65982 | 14:2854696 | 65988 | 14:3015800 | 65989 | 14:3040833 | 65990 | 14:3040885 | 65994 | 14:3097950 |
| 65998 | 14:3147254 | 66005 | 14:3206294 | 66006 | 14:3212884 | 66007 | 14:3218403 | 66008 | 14:3218478 |
| 66009 | 14:3218557 | 66010 | 14:3221269 | 66011 | 14:3221338 | 66012 | 14:3239650 | 66013 | 14:3239696 |
| 66014 | 14:3239752 | 66015 | 14:3239754 | 66016 | 14:3242336 | 66017 | 14:3246999 | 66018 | 14:3247042 |
| 66019 | 14:3247052 | 66020 | 14:3278835 | 66021 | 14:3278838 | 66022 | 14:3284409 | 66023 | 14:3284439 |
| 66024 | 14:3298378 | 66025 | 14:3305527 | 66026 | 14:3308936 | 66028 | 14:3336128 | 66029 | 14:3336263 |
| 66034 | 14:3386889 | 66036 | 14:3433819 | 66038 | 14:3453301 | 66041 | 14:3471847 | 66080 | 14:4216507 |
| 66082 | 14:4228026 | 66083 | 14:4228352 | 66085 | 14:4232919 | 66086 | 14:4233543 | 66113 | 14:4912069 |
| 66118 | 14:5004897 | 66178 | 14:5998302 | 66185 | 14:6071169 | 66191 | 14:6163665 | 66192 | 14:6174543 |
| 66203 | 14:6285204 | 66206 | 14:6295145 | 66207 | 14:6303201 | 66212 | 14:6388522 | 66214 | 14:6472930 |
| 66237 | 14:6859614 | 66248 | 14:7064392 | 66250 | 14:7109542 | 66252 | 14:7127144 | 66269 | 14:7497044 |
| 66274 | 14:7545925 | 66275 | 14:7545927 | 66276 | 14:7545936 | 66280 | 14:7626758 | 66294 | 14:7895151 |
| 66300 | 14:7989733 | 66311 | 14:8058621 | 66312 | 14:8058649 | 66316 | 14:8179389 | 66321 | 14:8301355 |
| 66323 | 14:8312764 | 66327 | 14:8394739 | 66328 | 14:8416607 | 66329 | 14:8424727 | 66331 | 14:8441111 |
| 66333 | 14:8445298 | 66337 | 14:8540399 | 66338 | 14:8551277 | 66340 | 14:8561525 | 66341 | 14:8566274 |
| 66343 | 14:8579200 | 66346 | 14:8601680 | 66347 | 14:8601681 | 66349 | 14:8629638 | 66353 | 14:8664840 |
| 66354 | 14:8664847 | 66356 | 14:8672236 | 66359 | 14:8707161 | 66363 | 14:8798924 | 66375 | 14:9035591 |
| 66378 | 14:9063069 | 66379 | 14:9064094 | 66380 | 14:9064111 | 66381 | 14:9064154 | 66382 | 14:9064258 |
| 66384 | 14:9095754 | 66386 | 14:9121381 | 66387 | 14:9140334 | 66388 | 14:9140361 | 66389 | 14:9140418 |
| 66390 | 14:9141498 | 66394 | 14:9188175 | 66400 | 14:9361706 | 66402 | 14:9382539 | 66403 | 14:9399137 |
| 66404 | 14:9401991 | 66430 | 14:9976682 | 66431 | 14:9976704 | 66432 | 14:9976709 | 66434 | 14:10038814 |
| 66438 | 14:10102756 | 66440 | 14:10152585 | 66443 | 14:10280749 | 66451 | 14:10486501 | 66455 | 14:10530506 |
| 66457 | 14:10557006 | 66458 | 14:10566461 | 66459 | 14:10577632 | 66462 | 14:10706026 | 66464 | 14:10756790 |
| 66465 | 14:10764173 | 66466 | 14:10788680 | 66467 | 14:10801527 | 66468 | 14:10805790 | 66469 | 14:10807806 |
| 66470 | 14:10816880 | 66474 | 14:10893686 | 66476 | 14:10914761 | 66479 | 14:10947558 | 66481 | 14:11009369 |
| 66484 | 14:11088275 | 66492 | 14:11252807 | 66504 | 14:11411289 | 66505 | 14:11444087 | 66510 | 14:11540331 |
| 66512 | 14:11568465 | 66513 | 14:11575517 | 66516 | 14:11596900 | 66517 | 14:11657154 | 66526 | 14:11763629 |
| 66533 | 14:11962212 | 66541 | 14:12136166 | 66546 | 14:12207418 | 66551 | 14:12292359 | 66552 | 14:12293976 |
| 66554 | 14:12315574 | 66558 | 14:12384353 | 66559 | 14:12388961 | 66560 | 14:12393160 | 66564 | 14:12514540 |
| 66578 | 14:12695561 | 66579 | 14:12710347 | 66593 | 14:13094541 | 66595 | 14:13178679 | 66618 | 14:13920366 |
| 66623 | 14:14023534 | 66628 | 14:14069858 | 66631 | 14:14135529 | 66636 | 14:14326793 | 66646 | 14:14560727 |
| 66649 | 14:14707903 | 66651 | 14:14728697 | 66652 | 14:14744411 | 66656 | 14:14812773 | 66657 | 14:14823932 |
| 66662 | 14:14952020 | 66664 | 14:15013585 | 66666 | 14:15072256 | 66668 | 14:15100414 | 66675 | 14:15318937 |
| 66677 | 14:15351351 | 66678 | 14:15369492 | 66682 | 14:15454870 | 66689 | 14:15529862 | 66704 | 14:15827253 |
| 66710 | 14:15926072 | 66712 | 14:15954837 | 66715 | 14:15972218 | 66723 | 14:16143522 | 66725 | 14:16177691 |
| 66730 | 14:16258523 | 66734 | 14:16293114 | 66737 | 14:16436394 | 66738 | 14:16442429 | 66739 | 14:16450949 |
| 66741 | 14:16479769 | 66742 | 14:16493894 | 66752 | 14:16722564 | 66753 | 14:16790460 | 66754 | 14:16817518 |
| 66756 | 14:16852507 | 66761 | 14:16906633 | 66762 | 14:16939530 | 66763 | 14:16939542 | 66764 | 14:16939598 |
| 66765 | 14:16939628 | 66767 | 14:16969260 | 66771 | 14:17041215 | 66778 | 14:17280279 | 66798 | 14:17770696 |
| 66799 | 14:17770830 | 66801 | 14:17799708 | 66802 | 14:17816110 | 66803 | 14:17843985 | 66836 | 14:18601570 |
| 66849 | 14:18880871 | 66860 | 14:19181912 | 66865 | 14:19283893 | 66869 | 14:19432077 | 66881 | 14:19699930 |
| 66882 | 14:19743977 | 66885 | 14:19813853 | 66890 | 14:19975802 | 66893 | 14:19991875 | 66927 | 14:20847256 |
| 66930 | 14:20893054 | 66931 | 14:20896371 | 66932 | 14:20913913 | 66935 | 14:20978169 | 66937 | 14:21048592 |
| 66958 | 14:21485350 | 66963 | 14:21604476 | 66974 | 14:21869418 | 66977 | 14:21931720 | 66979 | 14:21998392 |
| 66984 | 14:22161961 | 66995 | 14:22407071 | 66996 | 14:22407076 | 67077 | 14:22732607 | 67093 | 14:22882106 |
| 67095 | 14:22886422 | 67173 | 14:23215974 | 67345 | 14:23539160 | 67346 | 14:23539175 | 67347 | 14:23539193 |
| 67348 | 14:23539196 | 67349 | 14:23539229 | 67424 | 14:23981200 | 67428 | 14:24041313 | 67443 | 14:24389626 |
| 67445 | 14:24408833 | 67514 | 14:25111427 | 67519 | 14:25203874 | 67520 | 14:25205074 | 67525 | 14:25258907 |
| 67529 | 14:25432157 | 67561 | 14:25864255 | 67565 | 14:25923336 | 67566 | 14:25923373 | 67568 | 14:25985806 |
| 67570 | 14:25990906 | 67571 | 14:26012359 | 67573 | 14:26052398 | 67575 | 14:26065694 | 67580 | 14:26140450 |
| 67581 | 14:26140990 | 67582 | 14:26141962 | 67583 | 14:26143779 | 67585 | 14:26163203 | 67586 | 14:26170631 |
| 67587 | 14:26175012 | 67588 | 14:26176715 | 67590 | 14:26182412 | 67591 | 14:26182943 | 67593 | 14:26184111 |
| 67594 | 14:26184775 | 67595 | 14:26185527 | 67596 | 14:26186351 | 67597 | 14:26190407 | 67598 | 14:26191236 |
| 67600 | 14:26193748 | 67601 | 14:26194782 | 67602 | 14:26195880 | 67603 | 14:26197356 | 67604 | 14:26209375 |
| 67605 | 14:26210727 | 67606 | 14:26212648 | 67607 | 14:26216203 | 67608 | 14:26225153 | 67610 | 14:26246778 |
| 67611 | 14:26271742 | 67614 | 14:26308735 | 67617 | 14:26333506 | 67620 | 14:26432685 | 67626 | 14:26633301 |
| 67632 | 14:26717685 | 67648 | 14:27181625 | 67649 | 14:27210120 | 67650 | 14:27233742 | 67670 | 14:27952519 |
| 67673 | 14:28069983 | 67685 | 14:28479844 | 67691 | 14:28620485 | 67692 | 14:28622173 | 67693 | 14:28624764 |
| 67697 | 14:28758622 | 67715 | 14:29296020 | 67725 | 14:29459640 | 67740 | 14:29698665 | 67748 | 14:29894372 |
| 67749 | 14:29989315 | 67752 | 14:30012586 | 67767 | 14:30342088 | 67768 | 14:30349359 | 67769 | 14:30356890 |
| 67778 | 14:30465481 | 67781 | 14:30499744 | 67794 | 14:30690571 | 67797 | 14:30764962 | 67798 | 14:30811197 |
| 67808 | 14:31180467 | 67819 | 14:31555900 | 67820 | 14:31555992 | 67826 | 14:31641997 | 67833 | 14:31874739 |
| 67836 | 14:31944000 | 67844 | 14:32203109 | 67866 | 14:32398203 | 67886 | 14:32981178 | 67890 | 14:33060155 |
| 67896 | 14:33143197 | 67897 | 14:33143201 | 67900 | 14:33275732 | 67906 | 14:33326342 | 67907 | 14:33342080 |
| 67912 | 14:33449262 | 67913 | 14:33458276 | 67919 | 14:33529983 | 67930 | 14:33731278 | 67932 | 14:33839737 |
| 67938 | 14:33897507 | 67939 | 14:33921705 | 67942 | 14:33981693 | 67947 | 14:34164312 | 67953 | 14:34301611 |
| 67959 | 14:34528656 | 67975 | 14:35058048 | 68018 | 14:36043204 | 68028 | 14:36299508 | 68035 | 14:36373476 |
| 68052 | 14:36852980 | 68071 | 14:37488132 | 68084 | 14:37849675 | 68085 | 14:37852302 | 68089 | 14:37925483 |
| 68097 | 14:38188466 | 68115 | 14:38814239 | 68116 | 14:38815880 | 68117 | 14:38822128 | 68118 | 14:38822260 |
| 68141 | 14:39497949 | 68155 | 14:39876130 | 68156 | 14:39966401 | 68160 | 14:40176632 | 68161 | 14:40176648 |
| 68162 | 14:40176909 | 68163 | 14:40176917 | 68164 | 14:40176996 | 68165 | 14:40177014 | 68166 | 14:40177030 |
| 68167 | 14:40177236 | 68168 | 14:40177299 | 68179 | 14:40478861 | 68203 | 14:41361918 | 68205 | 14:41438833 |
| 68208 | 14:41573733 | 68214 | 14:41689230 | 68217 | 14:41730144 | 68218 | 14:41737864 | 68242 | 14:42286395 |
| 68254 | 14:42523669 | 68264 | 14:42805270 | 68273 | 14:43158811 | 68280 | 14:43440082 | 68288 | 14:43769450 |
| 68307 | 14:44309555 | 68309 | 14:44430721 | 68318 | 14:44636417 | 68322 | 14:44676672 | 68323 | 14:44676728 |
| 68324 | 14:44677183 | 68326 | 14:44690793 | 68327 | 14:44724666 | 68329 | 14:44762792 | 68331 | 14:44776625 |
| 68334 | 14:44843603 | 68335 | 14:44854809 | 68336 | 14:44856580 | 68340 | 14:44966227 | 68344 | 14:44988177 |
| 68345 | 14:45015405 | 68346 | 14:45018776 | 68347 | 14:45022201 | 68348 | 14:45041057 | 68356 | 14:45253129 |
| 68357 | 14:45264773 | 68362 | 14:45445260 | 68378 | 14:45823929 | 68379 | 14:45842554 | 68380 | 14:45845229 |
| 68387 | 14:45960737 | 68392 | 14:46043332 | 68395 | 14:46074189 | 68416 | 14:46394434 | 68419 | 14:46425925 |
| 68425 | 14:46627739 | 68427 | 14:46701293 | 68437 | 14:46893352 | 68447 | 14:47088081 | 68477 | 14:48079880 |
| 68486 | 14:48324535 | 68488 | 14:48324675 | 68491 | 14:48337571 | 68505 | 14:48683313 | 68508 | 14:48746367 |
| 68510 | 14:48777365 | 68531 | 14:49324283 | 68534 | 14:49446616 | 68535 | 14:49446636 | 68557 | 14:50162370 |
| 68562 | 14:50269979 | 68571 | 14:50641123 | 68586 | 14:51157346 | 68593 | 14:51380992 | 68594 | 14:51381093 |
| 68599 | 14:51513649 | 68609 | 14:51705820 | 68618 | 14:51969375 | 68621 | 14:52011701 | 68622 | 14:52087941 |
| 68624 | 14:52117812 | 68626 | 14:52174077 | 68627 | 14:52174084 | 68628 | 14:52176416 | 68629 | 14:52176503 |
| 68630 | 14:52181003 | 68631 | 14:52193756 | 68632 | 14:52206425 | 68634 | 14:52240902 | 68635 | 14:52240959 |
| 68636 | 14:52272882 | 68637 | 14:52274310 | 68647 | 14:52598698 | 68666 | 14:53395458 | 68667 | 14:53433645 |
| 68678 | 14:53716855 | 68694 | 14:54155951 | 68699 | 14:54289749 | 68703 | 14:54322218 | 68704 | 14:54323172 |
| 68705 | 14:54324711 | 68706 | 14:54326865 | 68707 | 14:54328568 | 68708 | 14:54329372 | 68709 | 14:54330950 |
| 68710 | 14:54331798 | 68711 | 14:54332484 | 68712 | 14:54333187 | 68713 | 14:54334454 | 68714 | 14:54335896 |
| 68715 | 14:54340206 | 68718 | 14:54366857 | 68719 | 14:54375801 | 68722 | 14:54397201 | 68723 | 14:54441985 |
| 68725 | 14:54458565 | 68732 | 14:54625281 | 68745 | 14:54834484 | 68749 | 14:55039582 | 68751 | 14:55074676 |
| 68756 | 14:55222131 | 68757 | 14:55226008 | 68758 | 14:55230280 | 68759 | 14:55230389 | 68760 | 14:55283357 |
| 68769 | 14:55484731 | 68780 | 14:55685950 | 68789 | 14:56225330 | 68790 | 14:56256482 | 68791 | 14:56276255 |
| 68799 | 14:56506951 | 68803 | 14:56545284 | 68804 | 14:56555253 | 68808 | 14:56652939 | 68816 | 14:56827541 |
| 68831 | 14:57332067 | 68832 | 14:57346169 | 68833 | 14:57350713 | 68834 | 14:57366372 | 68835 | 14:57383485 |
| 68839 | 14:57448890 | 68840 | 14:57459060 | 68842 | 14:57536798 | 68846 | 14:57585046 | 68851 | 14:57728888 |
| 68852 | 14:57728921 | 68853 | 14:57779780 | 68854 | 14:57796975 | 68856 | 14:57816918 | 68859 | 14:57922597 |
| 68860 | 14:57935303 | 68864 | 14:58056243 | 68868 | 14:58089515 | 68874 | 14:58220993 | 68885 | 14:58495696 |
| 68886 | 14:58510271 | 68888 | 14:58533542 | 68892 | 14:58668510 | 68905 | 14:58947933 | 68910 | 14:59026237 |
| 68912 | 14:59043151 | 68920 | 14:59231026 | 68947 | 14:59935092 | 68953 | 14:60142373 | 68957 | 14:60181604 |
| 68958 | 14:60181624 | 68964 | 14:60282711 | 68965 | 14:60298957 | 68967 | 14:60335923 | 68969 | 14:60400617 |
| 68973 | 14:60573159 | 68977 | 14:60646587 | 68978 | 14:60646600 | 68983 | 14:60827415 | 68990 | 14:60986477 |
| 68994 | 14:61146297 | 68996 | 14:61230816 | 68997 | 14:61230819 | 69001 | 14:61287942 | 69002 | 14:61313591 |
| 69003 | 14:61315627 | 69007 | 14:61411151 | 69008 | 14:61412641 | 69009 | 14:61412766 | 69010 | 14:61412805 |
| 69011 | 14:61414085 | 69014 | 14:61513794 | 69028 | 14:61777801 | 69033 | 14:61849353 | 69040 | 14:61948505 |
| 69044 | 14:62039648 | 69046 | 14:62081343 | 69049 | 14:62134019 | 69052 | 14:62144762 | 69053 | 14:62144767 |
| 69054 | 14:62149426 | 69060 | 14:62299231 | 69068 | 14:62442888 | 69073 | 14:62565653 | 69078 | 14:62687349 |
| 69088 | 14:62815477 | 69090 | 14:62881003 | 69100 | 14:63151822 | 69103 | 14:63198642 | 69104 | 14:63225917 |
| 69113 | 14:63395803 | 69128 | 14:63850266 | 69131 | 14:63928813 | 69146 | 14:64220537 | 69149 | 14:64336877 |
| 69150 | 14:64339120 | 69151 | 14:64353146 | 69156 | 14:64514888 | 69194 | 14:65705048 | 69201 | 14:65784254 |
| 69206 | 14:65890326 | 69211 | 14:66085117 | 69212 | 14:66121609 | 69213 | 14:66141319 | 69221 | 14:66266296 |
| 69222 | 14:66266410 | 69224 | 14:66350819 | 69225 | 14:66350850 | 69233 | 14:66424408 | 69237 | 14:66604661 |
| 69240 | 14:66662602 | 69241 | 14:66669902 | 69242 | 14:66669945 | 69247 | 14:66766263 | 69263 | 14:67192810 |
| 69265 | 14:67258930 | 69268 | 14:67326499 | 69270 | 14:67366402 | 69274 | 14:67469984 | 69275 | 14:67472732 |
| 69276 | 14:67473297 | 69277 | 14:67473813 | 69278 | 14:67478743 | 69279 | 14:67483782 | 69280 | 14:67495613 |
| 69284 | 14:67550636 | 69287 | 14:67603655 | 69290 | 14:67630731 | 69292 | 14:67655532 | 69293 | 14:67660678 |
| 69299 | 14:67907866 | 69318 | 14:68256914 | 69323 | 14:68358639 | 69355 | 14:69386387 | 69366 | 14:69774142 |
| 69367 | 14:69787949 | 69368 | 14:69802911 | 69371 | 14:69913935 | 69373 | 14:69940920 | 69375 | 14:70002759 |
| 69381 | 14:70075613 | 69385 | 14:70170918 | 69386 | 14:70171506 | 69388 | 14:70172986 | 69392 | 14:70218397 |
| 69398 | 14:70356280 | 69401 | 14:70440463 | 69403 | 14:70472924 | 69412 | 14:70696424 | 69418 | 14:70903085 |
| 69420 | 14:70964025 | 69440 | 14:71594264 | 69447 | 14:71762783 | 69459 | 14:72082157 | 69485 | 14:72739791 |
| 69489 | 14:72764165 | 69499 | 14:73108865 | 69504 | 14:73206090 | 69507 | 14:73232071 | 69510 | 14:73246685 |
| 69516 | 14:73444151 | 69525 | 14:73731378 | 69529 | 14:73806578 | 69578 | 14:75097229 | 69579 | 14:75097255 |
| 69582 | 14:75218040 | 69586 | 14:75379695 | 69587 | 14:75379698 | 69588 | 14:75379711 | 69592 | 14:75591751 |
| 69596 | 14:75706688 | 69611 | 14:76100504 | 69612 | 14:76100657 | 69615 | 14:76128821 | 69616 | 14:76129624 |
| 69617 | 14:76132580 | 69618 | 14:76138175 | 69619 | 14:76141064 | 69621 | 14:76142867 | 69622 | 14:76144495 |
| 69625 | 14:76178839 | 69626 | 14:76178890 | 69628 | 14:76195852 | 69629 | 14:76199476 | 69630 | 14:76207317 |
| 69635 | 14:76328357 | 69637 | 14:76366242 | 69646 | 14:76503558 | 69653 | 14:76612609 | 69655 | 14:76770661 |
| 69656 | 14:76771798 | 69659 | 14:76845625 | 69661 | 14:76923277 | 69663 | 14:76945033 | 69664 | 14:76964292 |
| 69670 | 14:77099484 | 69671 | 14:77174586 | 69672 | 14:77206279 | 69681 | 14:77471278 | 69682 | 14:77471456 |
| 69684 | 14:77512276 | 69712 | 14:78249703 | 69713 | 14:78249764 | 69714 | 14:78249780 | 69720 | 14:78305043 |
| 69721 | 14:78306690 | 69722 | 14:78312623 | 69725 | 14:78390822 | 69726 | 14:78392259 | 69727 | 14:78393807 |
| 69729 | 14:78454550 | 69738 | 14:78651480 | 69747 | 14:78858840 | 69755 | 14:79091368 | 69781 | 14:79790786 |
| 69787 | 14:79936016 | 69795 | 14:80077176 | 69796 | 14:80078158 | 69809 | 14:80313052 | 69832 | 14:80855743 |
| 69848 | 14:81221549 | 69849 | 14:81264509 | 69850 | 14:81267100 | 69851 | 14:81267190 | 69852 | 14:81267746 |
| 69853 | 14:81295824 | 69854 | 14:81295958 | 69861 | 14:81372244 | 69862 | 14:81379555 | 69863 | 14:81392217 |
| 69872 | 14:81555021 | 69873 | 14:81583416 | 69876 | 14:81598835 | 69879 | 14:81634869 | 69881 | 14:81683735 |
| 69882 | 14:81698012 | 69883 | 14:81701580 | 69887 | 14:81727910 | 69893 | 14:81835309 | 69895 | 14:81857929 |
| 69897 | 14:81940278 | 69899 | 14:81972242 | 69903 | 14:82015406 | 69904 | 14:82016702 | 69905 | 14:82026443 |
| 69909 | 14:82091345 | 69933 | 15:556424 | 69946 | 15:1148939 | 69948 | 15:1192605 | 69953 | 15:1229858 |
| 69961 | 15:1387083 | 69962 | 15:1439384 | 70009 | 15:2844618 | 70063 | 15:3928859 | 70079 | 15:4264863 |
| 70083 | 15:4294447 | 70092 | 15:4497670 | 70099 | 15:4629275 | 70119 | 15:4985484 | 70122 | 15:5160844 |
| 70123 | 15:5160845 | 70124 | 15:5160859 | 70125 | 15:5160876 | 70126 | 15:5160956 | 70128 | 15:5219937 |
| 70129 | 15:5234380 | 70130 | 15:5296961 | 70282 | 15:5477594 | 70286 | 15:5550405 | 70289 | 15:5646426 |
| 70292 | 15:5686808 | 70293 | 15:5686891 | 70294 | 15:5686893 | 70297 | 15:5779163 | 70300 | 15:5933216 |
| 70306 | 15:6041257 | 70320 | 15:6451524 | 70323 | 15:6555645 | 70324 | 15:6579715 | 70329 | 15:6707584 |
| 70374 | 15:7579129 | 70391 | 15:8102324 | 70413 | 15:8544147 | 70414 | 15:8547793 | 70425 | 15:8919907 |
| 70434 | 15:9119501 | 70444 | 15:9307296 | 70445 | 15:9307388 | 70446 | 15:9308875 | 70449 | 15:9342376 |
| 70461 | 15:9653267 | 70462 | 15:9653311 | 70466 | 15:9752248 | 70491 | 15:10716741 | 70493 | 15:10740715 |
| 70496 | 15:10827424 | 70505 | 15:11236303 | 70520 | 15:11770301 | 70529 | 15:12167647 | 70540 | 15:12557589 |
| 70574 | 15:13886436 | 70586 | 15:14183988 | 70588 | 15:14191992 | 70591 | 15:14203309 | 70594 | 15:14260966 |
| 70599 | 15:14448696 | 70600 | 15:14456572 | 70601 | 15:14458763 | 70602 | 15:14459519 | 70603 | 15:14461707 |
| 70604 | 15:14462057 | 70605 | 15:14462078 | 70606 | 15:14463666 | 70612 | 15:14705603 | 70617 | 15:14911197 |
| 70623 | 15:15068168 | 70624 | 15:15068209 | 70631 | 15:15245724 | 70643 | 15:15417315 | 70644 | 15:15417341 |
| 70645 | 15:15417537 | 70646 | 15:15417584 | 70648 | 15:15474272 | 70649 | 15:15505264 | 70651 | 15:15537455 |
| 70656 | 15:15691423 | 70657 | 15:15691442 | 70658 | 15:15715228 | 70661 | 15:15789950 | 70664 | 15:15846394 |
| 70665 | 15:15857275 | 70666 | 15:15867538 | 70667 | 15:15882003 | 70668 | 15:15887057 | 70671 | 15:15896498 |
| 70672 | 15:15896623 | 70673 | 15:15896626 | 70676 | 15:15899461 | 70678 | 15:15941852 | 70685 | 15:16025726 |
| 70686 | 15:16034972 | 70688 | 15:16066600 | 70689 | 15:16070995 | 70690 | 15:16086621 | 70691 | 15:16086825 |
| 70692 | 15:16090802 | 70693 | 15:16095590 | 70694 | 15:16098140 | 70697 | 15:16106288 | 70698 | 15:16106299 |
| 70699 | 15:16106352 | 70700 | 15:16106429 | 70701 | 15:16106956 | 70703 | 15:16181648 | 70720 | 15:16783559 |
| 70722 | 15:16876160 | 70725 | 15:17015197 | 70726 | 15:17021701 | 70727 | 15:17023074 | 70728 | 15:17023080 |
| 70729 | 15:17035605 | 70730 | 15:17045413 | 70731 | 15:17050219 | 70732 | 15:17051620 | 70734 | 15:17079996 |
| 70737 | 15:17110054 | 70739 | 15:17151448 | 70740 | 15:17156993 | 70742 | 15:17174949 | 70743 | 15:17174997 |
| 70744 | 15:17178030 | 70748 | 15:17216169 | 70749 | 15:17225710 | 70752 | 15:17272317 | 70771 | 15:17881167 |
| 70772 | 15:17905884 | 70773 | 15:17907768 | 70774 | 15:17907803 | 70776 | 15:17984528 | 70780 | 15:18132345 |
| 70788 | 15:18340406 | 70789 | 15:18340444 | 70790 | 15:18340452 | 70791 | 15:18340495 | 70792 | 15:18340522 |
| 70800 | 15:18582994 | 70828 | 15:19344195 | 70852 | 15:20040778 | 70853 | 15:20041510 | 70855 | 15:20059908 |
| 70861 | 15:20159086 | 70862 | 15:20208131 | 70863 | 15:20239028 | 70864 | 15:20251395 | 70874 | 15:20506266 |
| 70898 | 15:20943851 | 70900 | 15:20983899 | 70904 | 15:21079829 | 70909 | 15:21144577 | 70910 | 15:21222779 |
| 70915 | 15:21328250 | 70916 | 15:21331201 | 70917 | 15:21344247 | 70927 | 15:21713133 | 70928 | 15:21715125 |
| 70930 | 15:21724458 | 70937 | 15:21944261 | 70938 | 15:21955334 | 70939 | 15:21959233 | 70940 | 15:21980185 |
| 70944 | 15:22109125 | 70947 | 15:22219010 | 70950 | 15:22224493 | 70951 | 15:22255512 | 70959 | 15:22397563 |
| 70988 | 15:23032442 | 70992 | 15:23140676 | 70995 | 15:23230388 | 71002 | 15:23361381 | 71003 | 15:23382217 |
| 71006 | 15:23428462 | 71007 | 15:23428524 | 71008 | 15:23428581 | 71009 | 15:23495909 | 71023 | 15:23729866 |
| 71024 | 15:23732149 | 71059 | 15:23865919 | 71072 | 15:24238065 | 71074 | 15:24278519 | 71083 | 15:24394620 |
| 71084 | 15:24406959 | 71103 | 15:24778258 | 71104 | 15:24778597 | 71105 | 15:24783092 | 71145 | 15:25774695 |
| 71150 | 15:25820329 | 71157 | 15:25920672 | 71166 | 15:26130885 | 71170 | 15:26196504 | 71175 | 15:26276504 |
| 71176 | 15:26297137 | 71192 | 15:26653665 | 71199 | 15:26809489 | 71211 | 15:27151330 | 71219 | 15:27430624 |
| 71223 | 15:27474421 | 71226 | 15:27503920 | 71228 | 15:27522579 | 71230 | 15:27581168 | 71236 | 15:27795197 |
| 71240 | 15:27909371 | 71243 | 15:27975706 | 71246 | 15:28011102 | 71247 | 15:28029857 | 71248 | 15:28029959 |
| 71249 | 15:28047023 | 71251 | 15:28062942 | 71256 | 15:28137955 | 71257 | 15:28138113 | 71260 | 15:28217846 |
| 71261 | 15:28233069 | 71264 | 15:28261207 | 71276 | 15:28502744 | 71285 | 15:28668348 | 71286 | 15:28669780 |
| 71287 | 15:28674486 | 71288 | 15:28675386 | 71289 | 15:28678307 | 71290 | 15:28679179 | 71292 | 15:28683279 |
| 71293 | 15:28687052 | 71294 | 15:28689278 | 71304 | 15:28889609 | 71305 | 15:28903033 | 71306 | 15:28903036 |
| 71307 | 15:28904856 | 71311 | 15:28935265 | 71314 | 15:28955129 | 71315 | 15:28957203 | 71316 | 15:28995849 |
| 71318 | 15:29029067 | 71320 | 15:29051391 | 71327 | 15:29217795 | 71331 | 15:29377994 | 71338 | 15:29599939 |
| 71341 | 15:29669861 | 71342 | 15:29691482 | 71343 | 15:29695545 | 71344 | 15:29708525 | 71358 | 15:30156144 |
| 71359 | 15:30182678 | 71372 | 15:30495429 | 71376 | 15:30550572 | 71378 | 15:30571590 | 71379 | 15:30592484 |
| 71381 | 15:30681136 | 71387 | 15:30887853 | 71395 | 15:31076884 | 71398 | 15:31119240 | 71423 | 15:31752349 |
| 71426 | 15:31762344 | 71427 | 15:31762695 | 71428 | 15:31762716 | 71429 | 15:31762815 | 71430 | 15:31766858 |
| 71431 | 15:31772217 | 71432 | 15:31772367 | 71433 | 15:31772508 | 71435 | 15:31789881 | 71439 | 15:31967339 |
| 71447 | 15:32184560 | 71448 | 15:32184561 | 71455 | 15:32326526 | 71457 | 15:32408675 | 71458 | 15:32415848 |
| 71473 | 15:32775518 | 71475 | 15:32857841 | 71479 | 15:32885957 | 71481 | 15:32912616 | 71482 | 15:32919523 |
| 71487 | 15:33030880 | 71493 | 15:33140018 | 71495 | 15:33140162 | 71496 | 15:33143682 | 71499 | 15:33200022 |
| 71502 | 15:33313146 | 71505 | 15:33357725 | 71513 | 15:33683094 | 71522 | 15:33855059 | 71546 | 15:34227669 |
| 71554 | 15:34378606 | 71558 | 15:34416916 | 71563 | 15:34514270 | 71564 | 15:34527315 | 71572 | 15:34677976 |
| 71574 | 15:34706081 | 71576 | 15:34775584 | 71578 | 15:34801602 | 71582 | 15:34866384 | 71583 | 15:34892433 |
| 71588 | 15:34974291 | 71591 | 15:35040731 | 71592 | 15:35049691 | 71598 | 15:35277152 | 71599 | 15:35337946 |
| 71604 | 15:35461378 | 71611 | 15:35646977 | 71612 | 15:35648952 | 71615 | 15:35707211 | 71617 | 15:35715226 |
| 71635 | 15:36132660 | 71636 | 15:36169165 | 71639 | 15:36183126 | 71640 | 15:36219316 | 71645 | 15:36321976 |
| 71655 | 15:36576380 | 71657 | 15:36603077 | 71678 | 15:37088135 | 71684 | 15:37186172 | 71706 | 15:37547916 |
| 71731 | 15:38265442 | 71738 | 15:38472609 | 71769 | 15:39119041 | 71776 | 15:39333095 | 71777 | 15:39347738 |
| 71781 | 15:39412550 | 71785 | 15:39545268 | 71799 | 15:39871286 | 71804 | 15:39927546 | 71805 | 15:39927550 |
| 71806 | 15:39927601 | 71809 | 15:40067276 | 71812 | 15:40162512 | 71817 | 15:40279882 | 71818 | 15:40279893 |
| 71819 | 15:40279894 | 71820 | 15:40280012 | 71823 | 15:40449461 | 71824 | 15:40449484 | 71826 | 15:40502987 |
| 71832 | 15:40632071 | 71833 | 15:40641136 | 71835 | 15:40723287 | 71837 | 15:40759558 | 71838 | 15:40847130 |
| 71845 | 15:40955841 | 71849 | 15:41014898 | 71850 | 15:41016306 | 71852 | 15:41150753 | 71860 | 15:41290689 |
| 71869 | 15:41440324 | 71877 | 15:41698377 | 71878 | 15:41698492 | 71879 | 15:41698511 | 71880 | 15:41705974 |
| 71881 | 15:41705975 | 71882 | 15:41706097 | 71885 | 15:41765753 | 71886 | 15:41767083 | 71887 | 15:41768163 |
| 71889 | 15:41814640 | 71901 | 15:42032808 | 71904 | 15:42124927 | 71907 | 15:42185672 | 71909 | 15:42231274 |
| 71912 | 15:42264995 | 71924 | 15:42509261 | 71926 | 15:42590389 | 71932 | 15:42752352 | 71934 | 15:42787098 |
| 71936 | 15:42818617 | 71939 | 15:42848256 | 71940 | 15:42854523 | 71951 | 15:43042213 | 71952 | 15:43045041 |
| 71953 | 15:43046535 | 71954 | 15:43049707 | 71955 | 15:43053427 | 71956 | 15:43055412 | 71957 | 15:43056476 |
| 71958 | 15:43057569 | 71959 | 15:43058822 | 71960 | 15:43059592 | 71962 | 15:43063078 | 71963 | 15:43063710 |
| 71964 | 15:43097237 | 71965 | 15:43098155 | 71970 | 15:43259917 | 71971 | 15:43260963 | 71972 | 15:43262025 |
| 71973 | 15:43262890 | 71974 | 15:43269560 | 71975 | 15:43276395 | 71976 | 15:43286382 | 71977 | 15:43288762 |
| 71978 | 15:43292368 | 71979 | 15:43293082 | 71980 | 15:43294086 | 71989 | 15:43476234 | 71993 | 15:43560696 |
| 71994 | 15:43561701 | 71995 | 15:43563456 | 71996 | 15:43566385 | 71997 | 15:43573222 | 71998 | 15:43580011 |
| 72000 | 15:43625295 | 72002 | 15:43642580 | 72003 | 15:43664227 | 72007 | 15:43741512 | 72020 | 15:43936008 |
| 72022 | 15:43939348 | 72023 | 15:43939353 | 72024 | 15:43939488 | 72046 | 15:44460580 | 72047 | 15:44479177 |
| 72056 | 15:44643217 | 72058 | 15:44735861 | 72075 | 15:45129015 | 72083 | 15:45210490 | 72084 | 15:45221891 |
| 72085 | 15:45246954 | 72088 | 15:45346073 | 72090 | 15:45379221 | 72091 | 15:45415112 | 72103 | 15:45716064 |
| 72104 | 15:45722551 | 72109 | 15:45827220 | 72126 | 15:46426137 | 72128 | 15:46464105 | 72131 | 15:46490284 |
| 72133 | 15:46492960 | 72134 | 15:46494185 | 72135 | 15:46500386 | 72136 | 15:46500459 | 72137 | 15:46500629 |
| 72138 | 15:46500752 | 72139 | 15:46505669 | 72140 | 15:46506092 | 72141 | 15:46506887 | 72142 | 15:46506889 |
| 72143 | 15:46506939 | 72144 | 15:46507314 | 72145 | 15:46507929 | 72146 | 15:46508001 | 72147 | 15:46508021 |
| 72148 | 15:46512024 | 72149 | 15:46512113 | 72156 | 15:46613300 | 72159 | 15:46636679 | 72161 | 15:46645269 |
| 72162 | 15:46646092 | 72173 | 15:46864410 | 72174 | 15:46866871 | 72178 | 15:46967802 | 72189 | 15:47187912 |
| 72190 | 15:47188138 | 72191 | 15:47188507 | 72201 | 15:47615122 | 72202 | 15:47641029 | 72205 | 15:47779635 |
| 72206 | 15:47779640 | 72207 | 15:47779664 | 72208 | 15:47779674 | 72209 | 15:47787011 | 72210 | 15:47787118 |
| 72211 | 15:47848072 | 72212 | 15:47856278 | 72213 | 15:47857368 | 72215 | 15:47862235 | 72216 | 15:47862345 |
| 72217 | 15:47867091 | 72222 | 15:48008735 | 72224 | 15:48040175 | 72225 | 15:48040515 | 72226 | 15:48040954 |
| 72228 | 15:48078030 | 72229 | 15:48078755 | 72230 | 15:48102626 | 72231 | 15:48102703 | 72232 | 15:48103271 |
| 72233 | 15:48103402 | 72250 | 15:48373937 | 72251 | 15:48378524 | 72252 | 15:48386761 | 72253 | 15:48390883 |
| 72254 | 15:48396110 | 72255 | 15:48397484 | 72266 | 15:48867306 | 72267 | 15:48952912 | 72275 | 15:49295120 |
| 72287 | 15:49686635 | 72289 | 15:49798310 | 72292 | 15:49812025 | 72296 | 15:50073269 | 72302 | 15:50348347 |
| 72305 | 15:50405124 | 72306 | 15:50418732 | 72311 | 15:50513682 | 72312 | 15:50513924 | 72326 | 15:51057213 |
| 72329 | 15:51193044 | 72332 | 15:51271630 | 72333 | 15:51275342 | 72336 | 15:51360447 | 72337 | 15:51360592 |
| 72338 | 15:51362026 | 72339 | 15:51362043 | 72340 | 15:51362061 | 72341 | 15:51362080 | 72342 | 15:51362276 |
| 72343 | 15:51362277 | 72344 | 15:51362431 | 72345 | 15:51362457 | 72346 | 15:51362458 | 72347 | 15:51364116 |
| 72348 | 15:51364137 | 72349 | 15:51420115 | 72350 | 15:51441064 | 72351 | 15:51441095 | 72352 | 15:51511445 |
| 72354 | 15:51518000 | 72355 | 15:51519061 | 72357 | 15:51521753 | 72358 | 15:51523084 | 72359 | 15:51529172 |
| 72360 | 15:51530078 | 72361 | 15:51535001 | 72362 | 15:51542928 | 72366 | 15:51558105 | 72367 | 15:51563195 |
| 72370 | 15:51568569 | 72371 | 15:51576564 | 72372 | 15:51610810 | 72376 | 15:51684740 | 72377 | 15:51687709 |
| 72379 | 15:51689738 | 72382 | 15:51736976 | 72384 | 15:51790467 | 72386 | 15:51803577 | 72394 | 15:51988641 |
| 72397 | 15:52007899 | 72404 | 15:52185081 | 72406 | 15:52200879 | 72413 | 15:52360124 | 72424 | 15:52658762 |
| 72425 | 15:52660106 | 72426 | 15:52673691 | 72441 | 15:53067270 | 72445 | 15:53137213 | 72449 | 15:53308023 |
| 72451 | 15:53309880 | 72452 | 15:53310424 | 72453 | 15:53311034 | 72454 | 15:53311897 | 72455 | 15:53312462 |
| 72456 | 15:53314094 | 72457 | 15:53314682 | 72458 | 15:53316670 | 72459 | 15:53319540 | 72460 | 15:53320533 |
| 72461 | 15:53321307 | 72462 | 15:53323633 | 72463 | 15:53324367 | 72465 | 15:53325750 | 72466 | 15:53326273 |
| 72467 | 15:53326849 | 72468 | 15:53327898 | 72469 | 15:53332122 | 72471 | 15:53333616 | 72472 | 15:53338788 |
| 72476 | 15:53398368 | 72478 | 15:53441398 | 72486 | 15:53709788 | 72487 | 15:53727634 | 72488 | 15:53729356 |
| 72489 | 15:53738936 | 72490 | 15:53743609 | 72491 | 15:53753517 | 72492 | 15:53761189 | 72494 | 15:53788777 |
| 72495 | 15:53796595 | 72496 | 15:53800970 | 72502 | 15:53945334 | 72518 | 15:54255643 | 72527 | 15:54462755 |
| 72530 | 15:54489427 | 72531 | 15:54500400 | 72541 | 15:54748501 | 72544 | 15:54834057 | 72548 | 15:54885008 |
| 72549 | 15:54885114 | 72550 | 15:54885117 | 72551 | 15:54889719 | 72552 | 15:54889820 | 72556 | 15:55119302 |
| 72565 | 15:55289790 | 72569 | 15:55444251 | 72574 | 15:55529280 | 72582 | 15:55734507 | 72589 | 15:56022102 |
| 72596 | 15:56200579 | 72603 | 15:56497282 | 72604 | 15:56497450 | 72605 | 15:56501007 | 72607 | 15:56515635 |
| 72608 | 15:56515639 | 72609 | 15:56515642 | 72610 | 15:56515714 | 72611 | 15:56518845 | 72612 | 15:56518860 |
| 72613 | 15:56518880 | 72614 | 15:56518892 | 72615 | 15:56518948 | 72616 | 15:56519916 | 72617 | 15:56520003 |
| 72618 | 15:56520009 | 72619 | 15:56522354 | 72620 | 15:56523085 | 72621 | 15:56523145 | 72622 | 15:56525703 |
| 72623 | 15:56526321 | 72624 | 15:56527735 | 72625 | 15:56529668 | 72626 | 15:56548649 | 72644 | 15:56809212 |
| 72675 | 15:57003970 | 72684 | 15:57272924 | 72689 | 15:57423300 | 72692 | 15:57500020 | 72694 | 15:57536234 |
| 72695 | 15:57536250 | 72703 | 15:57718142 | 72709 | 15:57803493 | 72712 | 15:57854603 | 72722 | 15:58001428 |
| 72727 | 15:58088362 | 72734 | 15:58241375 | 72736 | 15:58282071 | 72737 | 15:58283033 | 72740 | 15:58330257 |
| 72760 | 15:58671427 | 72773 | 15:59112131 | 72787 | 15:59537850 | 72795 | 15:59796390 | 72809 | 15:60285268 |
| 72813 | 15:60368718 | 72815 | 15:60428854 | 72820 | 15:60523980 | 72823 | 15:60608578 | 72850 | 15:61257843 |
| 72854 | 15:61342496 | 72868 | 15:61603248 | 72869 | 15:61639615 | 72871 | 15:61721710 | 72872 | 15:61721767 |
| 72873 | 15:61724284 | 72874 | 15:61724369 | 72875 | 15:61724392 | 72876 | 15:61725702 | 72879 | 15:61767375 |
| 72894 | 15:62032399 | 72907 | 15:62467385 | 72912 | 15:62561316 | 72914 | 15:62563939 | 72915 | 15:62586048 |
| 72916 | 15:62586078 | 72920 | 15:62652293 | 72938 | 15:63066650 | 72940 | 15:63100087 | 72941 | 15:63107739 |
| 72949 | 15:63295827 | 72951 | 15:63300815 | 72952 | 15:63303147 | 72960 | 15:63529465 | 72962 | 15:63574887 |
| 72964 | 15:63579300 | 72969 | 15:63631630 | 72972 | 15:63722937 | 72973 | 15:63723070 | 72974 | 15:63743524 |
| 72979 | 15:63917098 | 72983 | 15:63967865 | 72997 | 15:64256628 | 72999 | 15:64283258 | 73001 | 15:64333153 |
| 73004 | 15:64416446 | 73023 | 15:64714146 | 73026 | 15:64744225 | 73027 | 15:64771598 | 73031 | 15:64801402 |
| 73033 | 15:64806923 | 73038 | 15:64854504 | 73040 | 15:64908292 | 73046 | 15:64986216 | 73050 | 15:65029608 |
| 73056 | 15:65148971 | 73064 | 15:65275286 | 73066 | 15:65335638 | 73075 | 15:65436899 | 73078 | 15:65521232 |
| 73080 | 15:65594811 | 73081 | 15:65607689 | 73083 | 15:65622127 | 73085 | 15:65656084 | 73086 | 15:65670390 |
| 73091 | 15:65822176 | 73093 | 15:65854403 | 73105 | 15:66145675 | 73106 | 15:66156197 | 73115 | 15:66337078 |
| 73117 | 15:66339107 | 73119 | 15:66380166 | 73121 | 15:66418487 | 73124 | 15:66460251 | 73127 | 15:66496173 |
| 73129 | 15:66543651 | 73141 | 15:66724079 | 73146 | 15:66881298 | 73154 | 15:67179102 | 73164 | 15:67470477 |
| 73176 | 15:67880702 | 73196 | 15:68313970 | 73199 | 15:68378183 | 73216 | 15:68982890 | 73217 | 15:69062551 |
| 73224 | 15:69173709 | 73249 | 15:70010700 | 73255 | 15:70308624 | 73259 | 15:70429364 | 73262 | 15:70465643 |
| 73282 | 15:71042092 | 73289 | 15:71280393 | 73299 | 15:71468201 | 73300 | 15:71468245 | 73315 | 15:71909279 |
| 73349 | 15:72924654 | 73350 | 15:72992522 | 73355 | 15:73041310 | 73358 | 15:73097589 | 73360 | 15:73124963 |
| 73362 | 15:73143263 | 73363 | 15:73144008 | 73364 | 15:73144009 | 73365 | 15:73144095 | 73366 | 15:73144112 |
| 73367 | 15:73144117 | 73368 | 15:73144142 | 73372 | 15:73231049 | 73373 | 15:73250550 | 73376 | 15:73270606 |
| 73385 | 15:73422749 | 73386 | 15:73470922 | 73389 | 15:73555524 | 73390 | 15:73555544 | 73391 | 15:73555546 |
| 73392 | 15:73555549 | 73394 | 15:73590486 | 73400 | 15:73747012 | 73407 | 15:74016858 | 73408 | 15:74019876 |
| 73409 | 15:74047524 | 73412 | 15:74101712 | 73413 | 15:74120008 | 73421 | 15:74204147 | 73422 | 15:74221807 |
| 73423 | 15:74236733 | 73440 | 15:74680300 | 73442 | 15:74706685 | 73443 | 15:74715602 | 73444 | 15:74730746 |
| 73445 | 15:74736252 | 73448 | 15:74763309 | 73449 | 15:74765962 | 73456 | 15:74857238 | 73476 | 15:75401306 |
| 73484 | 15:75520621 | 73496 | 15:75764247 | 73498 | 15:75807751 | 73505 | 15:76018207 | 73513 | 15:76268285 |
| 73514 | 15:76284024 | 73515 | 15:76295386 | 73517 | 15:76357310 | 73519 | 15:76369909 | 73520 | 15:76423356 |
| 73525 | 15:76490114 | 73530 | 15:76627029 | 73531 | 15:76628527 | 73533 | 15:76676572 | 73542 | 15:76909234 |
| 73543 | 15:76913709 | 73554 | 15:77213130 | 73558 | 15:77256270 | 73561 | 15:77333647 | 73563 | 15:77341921 |
| 73569 | 15:77457892 | 73570 | 15:77533291 | 73572 | 15:77565837 | 73573 | 15:77581726 | 73574 | 15:77581747 |
| 73575 | 15:77581752 | 73576 | 15:77581872 | 73577 | 15:77581882 | 73580 | 15:77638946 | 73582 | 15:77676086 |
| 73590 | 15:77841257 | 73591 | 15:77844974 | 73597 | 15:77988330 | 73598 | 15:77989577 | 73600 | 15:77997353 |
| 73601 | 15:78001161 | 73603 | 15:78001840 | 73604 | 15:78003499 | 73605 | 15:78004209 | 73606 | 15:78008228 |
| 73607 | 15:78010723 | 73608 | 15:78011919 | 73610 | 15:78017138 | 73613 | 15:78027580 | 73614 | 15:78028904 |
| 73615 | 15:78032066 | 73616 | 15:78033166 | 73617 | 15:78033926 | 73618 | 15:78036014 | 73619 | 15:78037096 |
| 73620 | 15:78043902 | 73621 | 15:78049025 | 73623 | 15:78065440 | 73624 | 15:78069489 | 73637 | 15:78497070 |
| 73643 | 15:78705678 | 73644 | 15:78711863 | 73654 | 15:79013310 | 73662 | 15:79316918 | 73663 | 15:79316919 |
| 73665 | 15:79446531 | 73666 | 15:79447076 | 73667 | 15:79447126 | 73668 | 15:79447145 | 73671 | 15:79651229 |
| 73672 | 15:79651253 | 73684 | 15:79994534 | 73699 | 15:80313710 | 73702 | 15:80329440 | 73707 | 15:80387982 |
| 73711 | 15:80447483 | 73714 | 15:80528280 | 73722 | 15:80779796 | 73724 | 15:80825322 | 73725 | 15:80846525 |
| 73728 | 15:80988789 | 73733 | 15:81116878 | 73735 | 15:81144733 | 73736 | 15:81149287 | 73737 | 15:81161372 |
| 73739 | 15:81168190 | 73744 | 15:81197998 | 73751 | 15:81267470 | 73752 | 15:81279270 | 73759 | 15:81372132 |
| 73775 | 15:81857983 | 73793 | 15:82380481 | 73799 | 15:82629156 | 73806 | 15:82720061 | 73807 | 15:82720089 |
| 73810 | 15:82745967 | 73811 | 15:82746237 | 73815 | 15:82788081 | 73817 | 15:82797551 | 73819 | 15:82843451 |
| 73820 | 15:82851394 | 73824 | 15:82933219 | 73825 | 15:82949122 | 73830 | 15:83006747 | 73831 | 15:83013517 |
| 73832 | 15:83013534 | 73840 | 15:83073328 | 73848 | 15:83277486 | 73849 | 15:83288242 | 73850 | 15:83309084 |
| 73855 | 15:83458586 | 73865 | 15:83567476 | 73866 | 15:83583523 | 73867 | 15:83586864 | 73868 | 15:83586930 |
| 73911 | 16:752827 | 73914 | 16:766023 | 73915 | 16:804997 | 73916 | 16:806537 | 73922 | 16:921175 |
| 73929 | 16:978613 | 73937 | 16:1181761 | 73939 | 16:1204788 | 73941 | 16:1214929 | 73946 | 16:1324936 |
| 73963 | 16:1582068 | 73964 | 16:1599816 | 73965 | 16:1625990 | 73988 | 16:2055227 | 73992 | 16:2194637 |
| 73993 | 16:2224824 | 73995 | 16:2248476 | 73996 | 16:2263389 | 73997 | 16:2277642 | 74004 | 16:2402507 |
| 74005 | 16:2467947 | 74013 | 16:2602159 | 74017 | 16:2743842 | 74023 | 16:2938620 | 74026 | 16:3018942 |
| 74030 | 16:3067157 | 74038 | 16:3201109 | 74040 | 16:3201263 | 74042 | 16:3253128 | 74043 | 16:3269182 |
| 74044 | 16:3269280 | 74045 | 16:3270297 | 74051 | 16:3378716 | 74055 | 16:3405375 | 74059 | 16:3465967 |
| 74062 | 16:3607615 | 74065 | 16:3643356 | 74066 | 16:3644483 | 74073 | 16:3791803 | 74080 | 16:3992289 |
| 74087 | 16:4106339 | 74088 | 16:4109307 | 74093 | 16:4171303 | 74099 | 16:4262626 | 74100 | 16:4262650 |
| 74101 | 16:4265692 | 74103 | 16:4290878 | 74109 | 16:4423692 | 74114 | 16:4475911 | 74119 | 16:4592258 |
| 74122 | 16:4640118 | 74125 | 16:4717716 | 74129 | 16:4872430 | 74130 | 16:4898040 | 74133 | 16:4999290 |
| 74140 | 16:5116140 | 74146 | 16:5243133 | 74147 | 16:5265259 | 74148 | 16:5265363 | 74162 | 16:6052639 |
| 74164 | 16:6060113 | 74166 | 16:6207254 | 74168 | 16:6308221 | 74169 | 16:6317267 | 74170 | 16:6318112 |
| 74171 | 16:6319483 | 74172 | 16:6323633 | 74173 | 16:6325179 | 74175 | 16:6332032 | 74176 | 16:6333222 |
| 74177 | 16:6335935 | 74180 | 16:6376238 | 74191 | 16:6749567 | 74204 | 16:7268343 | 74214 | 16:7503961 |
| 74270 | 16:9734287 | 74280 | 16:9996877 | 74307 | 16:10894806 | 74318 | 16:11281651 | 74336 | 16:11756836 |
| 74343 | 16:11874391 | 74347 | 16:11985702 | 74364 | 16:12483562 | 74365 | 16:12486013 | 74378 | 16:12756820 |
| 74383 | 16:12870981 | 74400 | 16:13072437 | 74411 | 16:13341826 | 74415 | 16:13479712 | 74431 | 16:13921125 |
| 74441 | 16:14274292 | 74444 | 16:14375476 | 74450 | 16:14512750 | 74456 | 16:14612740 | 74457 | 16:14612809 |
| 74485 | 16:15352405 | 74486 | 16:15355419 | 74514 | 16:16201608 | 74519 | 16:16433665 | 74520 | 16:16433701 |
| 74532 | 16:16902875 | 74586 | 16:18664935 | 74596 | 16:18957732 | 74624 | 16:19622167 | 74629 | 16:19786076 |
| 74645 | 16:20175121 | 74649 | 16:20294926 | 74659 | 16:20540783 | 74660 | 16:20555295 | 74674 | 16:20905213 |
| 74676 | 16:20943232 | 74715 | 16:21954699 | 74722 | 16:22188120 | 74724 | 16:22316546 | 74726 | 16:22390334 |
| 74729 | 16:22420005 | 74742 | 16:22743613 | 74744 | 16:22775537 | 74745 | 16:22786666 | 74762 | 16:23286838 |
| 74768 | 16:23427214 | 74769 | 16:23439282 | 74770 | 16:23508581 | 74771 | 16:23518024 | 74772 | 16:23544281 |
| 74773 | 16:23557997 | 74776 | 16:23621715 | 74782 | 16:23717545 | 74783 | 16:23721462 | 74792 | 16:23915566 |
| 74793 | 16:23918537 | 74801 | 16:24174300 | 74802 | 16:24198154 | 74806 | 16:24253488 | 74809 | 16:24321784 |
| 74822 | 16:24770805 | 74844 | 16:24996364 | 74847 | 16:25007172 | 74856 | 16:25057711 | 74879 | 16:25209115 |
| 74900 | 16:25434176 | 74908 | 16:25723685 | 74909 | 16:25753006 | 74910 | 16:25760167 | 74911 | 16:25845363 |
| 74915 | 16:25999582 | 74916 | 16:26009236 | 74917 | 16:26012436 | 74918 | 16:26021774 | 74920 | 16:26036756 |
| 74921 | 16:26037402 | 74924 | 16:26139905 | 74925 | 16:26150072 | 74926 | 16:26150074 | 74927 | 16:26153413 |
| 74929 | 16:26186921 | 74930 | 16:26216113 | 74931 | 16:26217301 | 74933 | 16:26220845 | 74934 | 16:26227564 |
| 74935 | 16:26274073 | 74936 | 16:26296242 | 74940 | 16:26352124 | 74941 | 16:26356312 | 74943 | 16:26451726 |
| 74951 | 16:26637237 | 74973 | 16:27186026 | 74989 | 16:27756698 | 74996 | 16:27880735 | 74997 | 16:27880880 |
| 75002 | 16:27952368 | 75003 | 16:28008613 | 75004 | 16:28008626 | 75013 | 16:28274084 | 75024 | 16:28665499 |
| 75034 | 16:28945983 | 75036 | 16:29003100 | 75057 | 16:29692650 | 75059 | 16:29709436 | 75060 | 16:29760720 |
| 75061 | 16:29760768 | 75064 | 16:29872847 | 75065 | 16:29893692 | 75079 | 16:30287006 | 75081 | 16:30348202 |
| 75088 | 16:30604134 | 75092 | 16:30729515 | 75096 | 16:30772837 | 75098 | 16:30794250 | 75099 | 16:30795047 |
| 75100 | 16:30802513 | 75102 | 16:30839358 | 75108 | 16:31046070 | 75109 | 16:31054739 | 75120 | 16:31360463 |
| 75130 | 16:31587509 | 75131 | 16:31613864 | 75137 | 16:31734054 | 75146 | 16:31961054 | 75147 | 16:31961057 |
| 75151 | 16:32067908 | 75157 | 16:32356916 | 75162 | 16:32428255 | 75165 | 16:32550214 | 75180 | 16:32851343 |
| 75183 | 16:32875785 | 75184 | 16:32880400 | 75187 | 16:32951618 | 75188 | 16:32951739 | 75215 | 16:33649281 |
| 75216 | 16:33650436 | 75220 | 16:33885770 | 75224 | 16:33965943 | 75226 | 16:33984101 | 75247 | 16:34572625 |
| 75250 | 16:34620173 | 75254 | 16:34722052 | 75257 | 16:34852541 | 75262 | 16:34933617 | 75263 | 16:34933651 |
| 75267 | 16:35001017 | 75270 | 16:35137739 | 75272 | 16:35194830 | 75273 | 16:35194886 | 75274 | 16:35194897 |
| 75276 | 16:35202714 | 75277 | 16:35234653 | 75278 | 16:35244272 | 75279 | 16:35254252 | 75280 | 16:35290661 |
| 75281 | 16:35295775 | 75295 | 16:35792938 | 75302 | 16:35995859 | 75303 | 16:36000751 | 75304 | 16:36004313 |
| 75307 | 16:36081689 | 75313 | 16:36290108 | 75314 | 16:36299734 | 75317 | 16:36326781 | 75321 | 16:36365140 |
| 75330 | 16:36566768 | 75335 | 16:36697557 | 75336 | 16:36712618 | 75337 | 16:36775388 | 75344 | 16:36945543 |
| 75345 | 16:36969420 | 75365 | 16:37448884 | 75366 | 16:37448890 | 75376 | 16:37796926 | 75381 | 16:37913794 |
| 75382 | 16:37913795 | 75383 | 16:37913847 | 75384 | 16:37913888 | 75386 | 16:37937575 | 75397 | 16:38107328 |
| 75399 | 16:38122376 | 75401 | 16:38137107 | 75402 | 16:38150887 | 75406 | 16:38234762 | 75407 | 16:38244862 |
| 75423 | 16:38532788 | 75431 | 16:38885897 | 75458 | 16:39922839 | 75459 | 16:39929733 | 75482 | 16:40598638 |
| 75485 | 16:40704573 | 75500 | 16:41266328 | 75513 | 16:41835712 | 75514 | 16:41852637 | 75515 | 16:41881957 |
| 75516 | 16:41882451 | 75517 | 16:41882906 | 75518 | 16:41882957 | 75520 | 16:41883859 | 75521 | 16:41884250 |
| 75522 | 16:41889773 | 75523 | 16:41890000 | 75524 | 16:41892823 | 75525 | 16:41896465 | 75526 | 16:41898495 |
| 75527 | 16:41898551 | 75528 | 16:41908374 | 75529 | 16:41908542 | 75530 | 16:41909276 | 75531 | 16:41909349 |
| 75532 | 16:41909492 | 75533 | 16:41909626 | 75534 | 16:41909729 | 75535 | 16:41909886 | 75536 | 16:41929565 |
| 75537 | 16:41931893 | 75538 | 16:41931907 | 75539 | 16:41934619 | 75540 | 16:41934651 | 75541 | 16:41934687 |
| 75543 | 16:41947567 | 75544 | 16:41964938 | 75554 | 16:42253482 | 75557 | 16:42465089 | 75558 | 16:42547847 |
| 75559 | 16:42600403 | 75569 | 16:43061490 | 75575 | 16:43278476 | 75576 | 16:43330994 | 75578 | 16:43398900 |
| 75583 | 16:43745014 | 75593 | 16:44139005 | 75604 | 16:44403647 | 75606 | 16:44524286 | 75609 | 16:44660436 |
| 75610 | 16:44759709 | 75625 | 16:45375047 | 75641 | 16:46123551 | 75647 | 16:46250903 | 75649 | 16:46275222 |
| 75650 | 16:46286961 | 75651 | 16:46287045 | 75658 | 16:46502252 | 75665 | 16:46683276 | 75671 | 16:46787853 |
| 75672 | 16:46830087 | 75680 | 16:47040946 | 75682 | 16:47083406 | 75684 | 16:47094623 | 75685 | 16:47120517 |
| 75689 | 16:47196401 | 75697 | 16:47379730 | 75698 | 16:47379738 | 75702 | 16:47529414 | 75709 | 16:47649083 |
| 75710 | 16:47653055 | 75711 | 16:47658993 | 75719 | 16:47876641 | 75748 | 16:48471006 | 75749 | 16:48471012 |
| 75750 | 16:48471070 | 75767 | 16:48815391 | 75788 | 16:49290662 | 75798 | 16:49608069 | 75800 | 16:49640815 |
| 75804 | 16:49736666 | 75816 | 16:50023350 | 75856 | 16:51315511 | 75862 | 16:51572737 | 75866 | 16:51638935 |
| 75870 | 16:51814662 | 75871 | 16:51818542 | 75872 | 16:51823133 | 75873 | 16:51827521 | 75874 | 16:51828907 |
| 75875 | 16:51830294 | 75876 | 16:51830796 | 75877 | 16:51841289 | 75879 | 16:51854505 | 75881 | 16:51899632 |
| 75889 | 16:52062903 | 75892 | 16:52124016 | 75896 | 16:52189080 | 75897 | 16:52203550 | 75905 | 16:52391190 |
| 75906 | 16:52391213 | 75907 | 16:52401546 | 75908 | 16:52413488 | 75911 | 16:52492743 | 75920 | 16:52664549 |
| 75922 | 16:52715395 | 75933 | 16:53089976 | 75945 | 16:53432925 | 75951 | 16:53742380 | 75952 | 16:53742397 |
| 75953 | 16:53742405 | 75954 | 16:53742482 | 75960 | 16:53807690 | 75962 | 16:53819240 | 75971 | 16:54022291 |
| 75972 | 16:54037567 | 75973 | 16:54047358 | 76003 | 16:54893701 | 76004 | 16:54916866 | 76005 | 16:54917648 |
| 76008 | 16:54974984 | 76009 | 16:54975864 | 76010 | 16:55000694 | 76011 | 16:55015703 | 76012 | 16:55021774 |
| 76013 | 16:55109042 | 76032 | 16:55624726 | 76041 | 16:55875606 | 76053 | 16:56173257 | 76055 | 16:56193959 |
| 76056 | 16:56206450 | 76066 | 16:56367576 | 76067 | 16:56372228 | 76069 | 16:56383292 | 76072 | 16:56444611 |
| 76077 | 16:56546719 | 76080 | 16:56571482 | 76081 | 16:56571485 | 76082 | 16:56571494 | 76089 | 16:56837687 |
| 76096 | 16:57122424 | 76100 | 16:57326320 | 76101 | 16:57329788 | 76113 | 16:57586740 | 76114 | 16:57608446 |
| 76126 | 16:57873879 | 76129 | 16:58011779 | 76130 | 16:58012586 | 76146 | 16:58359793 | 76156 | 16:58464078 |
| 76158 | 16:58524073 | 76175 | 16:58904562 | 76176 | 16:58904595 | 76199 | 16:59323799 | 76201 | 16:59330153 |
| 76211 | 16:59665282 | 76212 | 16:59689730 | 76213 | 16:59689802 | 76222 | 16:59946371 | 76231 | 16:60201651 |
| 76233 | 16:60224702 | 76234 | 16:60224712 | 76236 | 16:60251070 | 76237 | 16:60253242 | 76238 | 16:60254578 |
| 76240 | 16:60311442 | 76241 | 16:60322616 | 76242 | 16:60322634 | 76245 | 16:60357272 | 76247 | 16:60391026 |
| 76251 | 16:60558830 | 76258 | 16:60784981 | 76274 | 16:61120937 | 76279 | 16:61322631 | 76285 | 16:61383011 |
| 76286 | 16:61387337 | 76301 | 16:61731673 | 76306 | 16:61957053 | 76312 | 16:62071218 | 76313 | 16:62072068 |
| 76314 | 16:62072151 | 76315 | 16:62072468 | 76316 | 16:62077343 | 76318 | 16:62094667 | 76319 | 16:62094728 |
| 76327 | 16:62245438 | 76328 | 16:62267314 | 76329 | 16:62275255 | 76335 | 16:62455462 | 76343 | 16:62633633 |
| 76348 | 16:62852449 | 76349 | 16:62856876 | 76354 | 16:62916406 | 76356 | 16:62926757 | 76369 | 16:63226438 |
| 76370 | 16:63226475 | 76371 | 16:63252161 | 76372 | 16:63264840 | 76374 | 16:63283115 | 76376 | 16:63353590 |
| 76382 | 16:63498489 | 76389 | 16:63665761 | 76391 | 16:63691003 | 76395 | 16:63806226 | 76397 | 16:63832124 |
| 76403 | 16:63910273 | 76404 | 16:63918137 | 76406 | 16:63985213 | 76413 | 16:64162147 | 76421 | 16:64386831 |
| 76426 | 16:64477575 | 76427 | 16:64478870 | 76428 | 16:64480600 | 76429 | 16:64482426 | 76430 | 16:64483727 |
| 76431 | 16:64486623 | 76432 | 16:64488080 | 76434 | 16:64560495 | 76437 | 16:64585141 | 76438 | 16:64587028 |
| 76439 | 16:64599607 | 76447 | 16:64716072 | 76457 | 16:64911089 | 76458 | 16:64915512 | 76459 | 16:64917512 |
| 76460 | 16:64917539 | 76461 | 16:64920273 | 76462 | 16:64924645 | 76463 | 16:64945487 | 76464 | 16:64947395 |
| 76466 | 16:64979912 | 76467 | 16:64979914 | 76468 | 16:64979920 | 76469 | 16:64980083 | 76473 | 16:65100643 |
| 76479 | 16:65256823 | 76480 | 16:65263949 | 76483 | 16:65320518 | 76490 | 16:65492703 | 76491 | 16:65500984 |
| 76492 | 16:65508159 | 76493 | 16:65511465 | 76494 | 16:65514812 | 76495 | 16:65518974 | 76496 | 16:65521306 |
| 76505 | 16:65841167 | 76512 | 16:66020681 | 76515 | 16:66100353 | 76517 | 16:66121444 | 76521 | 16:66169638 |
| 76523 | 16:66271884 | 76532 | 16:66520379 | 76533 | 16:66528712 | 76536 | 16:66553762 | 76538 | 16:66566651 |
| 76550 | 16:66804665 | 76551 | 16:66825677 | 76553 | 16:66859242 | 76555 | 16:66896436 | 76557 | 16:66919773 |
| 76558 | 16:66919950 | 76564 | 16:67050756 | 76565 | 16:67108343 | 76571 | 16:67259430 | 76573 | 16:67296635 |
| 76580 | 16:67413442 | 76581 | 16:67414503 | 76582 | 16:67414576 | 76583 | 16:67414591 | 76584 | 16:67418245 |
| 76588 | 16:67495932 | 76599 | 16:67610069 | 76610 | 16:67976663 | 76611 | 16:67981588 | 76626 | 16:68435498 |
| 76627 | 16:68435550 | 76636 | 16:68624997 | 76652 | 16:69066498 | 76653 | 16:69066625 | 76656 | 16:69080402 |
| 76657 | 16:69086868 | 76660 | 16:69129862 | 76665 | 16:69230127 | 76671 | 16:69350387 | 76679 | 16:69497807 |
| 76680 | 16:69501212 | 76681 | 16:69541904 | 76685 | 16:69607606 | 76688 | 16:69676831 | 76689 | 16:69704477 |
| 76692 | 16:69802000 | 76719 | 16:70380777 | 76720 | 16:70381368 | 76730 | 16:70587239 | 76731 | 16:70590791 |
| 76732 | 16:70590919 | 76733 | 16:70608188 | 76744 | 16:70871474 | 76754 | 16:71132906 | 76759 | 16:71385575 |
| 76762 | 16:71432921 | 76766 | 16:71483613 | 76771 | 16:71716120 | 76772 | 16:71732153 | 76777 | 16:71834818 |
| 76784 | 16:71964464 | 76785 | 16:71974676 | 76792 | 16:72212208 | 76797 | 16:72370528 | 76798 | 16:72370550 |
| 76799 | 16:72370562 | 76800 | 16:72370636 | 76803 | 16:72382117 | 76804 | 16:72382163 | 76805 | 16:72382236 |
| 76806 | 16:72382280 | 76808 | 16:72394792 | 76811 | 16:72421917 | 76813 | 16:72440326 | 76814 | 16:72473155 |
| 76815 | 16:72473197 | 76821 | 16:72619958 | 76823 | 16:72642068 | 76826 | 16:72684703 | 76828 | 16:72684778 |
| 76829 | 16:72684811 | 76831 | 16:72698852 | 76832 | 16:72700502 | 76833 | 16:72701289 | 76848 | 16:73201335 |
| 76853 | 16:73303554 | 76860 | 16:73466388 | 76862 | 16:73504205 | 76865 | 16:73518067 | 76866 | 16:73534249 |
| 76868 | 16:73563070 | 76869 | 16:73563134 | 76871 | 16:73583302 | 76873 | 16:73591363 | 76874 | 16:73597712 |
| 76876 | 16:73599978 | 76877 | 16:73607828 | 76878 | 16:73622925 | 76879 | 16:73694168 | 76880 | 16:73694195 |
| 76881 | 16:73694803 | 76882 | 16:73697167 | 76883 | 16:73697204 | 76884 | 16:73697206 | 76885 | 16:73697258 |
| 76886 | 16:73697700 | 76887 | 16:73699147 | 76889 | 16:73715301 | 76892 | 16:73750079 | 76909 | 16:74262736 |
| 76910 | 16:74267964 | 76924 | 16:74548368 | 76931 | 16:74775935 | 76936 | 16:74853499 | 76945 | 16:75005400 |
| 76946 | 16:75005468 | 76948 | 16:75032047 | 76953 | 16:75118546 | 76954 | 16:75132927 | 76955 | 16:75142495 |
| 76956 | 16:75143544 | 76957 | 16:75153428 | 76959 | 16:75202467 | 76960 | 16:75217027 | 76963 | 16:75248051 |
| 76965 | 16:75255731 | 76966 | 16:75256528 | 76967 | 16:75273493 | 76971 | 16:75346062 | 76978 | 16:75476985 |
| 76979 | 16:75477188 | 76980 | 16:75487044 | 76986 | 16:75656899 | 76991 | 16:75736008 | 76996 | 16:75817486 |
| 77008 | 16:76064461 | 77010 | 16:76082386 | 77016 | 16:76264493 | 77020 | 16:76316949 | 77023 | 16:76382298 |
| 77025 | 16:76417156 | 77026 | 16:76424214 | 77029 | 16:76490659 | 77034 | 16:76550668 | 77035 | 16:76561525 |
| 77038 | 16:76632577 | 77039 | 16:76645921 | 77044 | 16:76724147 | 77047 | 16:76749641 | 77049 | 16:76810446 |
| 77050 | 16:76898157 | 77053 | 16:77026179 | 77066 | 16:77196110 | 77075 | 16:77345710 | 77076 | 16:77345711 |
| 77086 | 16:77672463 | 77088 | 16:77700431 | 77133 | 16:78730933 | 77136 | 16:78794726 | 77146 | 16:78936706 |
| 77147 | 16:78936848 | 77148 | 16:78936900 | 77162 | 16:79072375 | 77170 | 16:79207667 | 77171 | 16:79214144 |
| 77172 | 16:79214863 | 77173 | 16:79214898 | 77174 | 16:79216805 | 77175 | 16:79216824 | 77176 | 16:79216879 |
| 77177 | 16:79217281 | 77178 | 16:79246839 | 77179 | 16:79247022 | 77180 | 16:79248848 | 77181 | 16:79252434 |
| 77182 | 16:79252455 | 77183 | 16:79253093 | 77184 | 16:79253212 | 77185 | 16:79253954 | 77186 | 16:79256053 |
| 77187 | 16:79258337 | 77188 | 16:79258375 | 77189 | 16:79259607 | 77190 | 16:79259608 | 77191 | 16:79259624 |
| 77192 | 16:79259664 | 77193 | 16:79259693 | 77194 | 16:79264907 | 77195 | 16:79264964 | 77196 | 16:79265514 |
| 77197 | 16:79265600 | 77199 | 16:79316462 | 77203 | 16:79347181 | 77206 | 16:79436693 | 77207 | 16:79449942 |
| 77208 | 16:79458180 | 77211 | 16:79513573 | 77212 | 16:79514562 | 77213 | 16:79526906 | 77215 | 16:79554621 |
| 77216 | 16:79563778 | 77217 | 16:79626644 | 77219 | 16:79659075 | 77223 | 16:79862681 | 77224 | 16:79881468 |
| 77225 | 16:79889426 | 77228 | 16:80019106 | 77231 | 16:80220495 | 77234 | 16:80431031 | 77240 | 16:80869601 |
| 77259 | 17:425858 | 77280 | 17:1125457 | 77282 | 17:1162137 | 77283 | 17:1162139 | 77284 | 17:1162184 |
| 77285 | 17:1162199 | 77286 | 17:1162200 | 77300 | 17:1367292 | 77306 | 17:1539162 | 77316 | 17:1782217 |
| 77338 | 17:2449957 | 77357 | 17:2912361 | 77367 | 17:3145731 | 77376 | 17:3382377 | 77391 | 17:3546676 |
| 77399 | 17:3801647 | 77403 | 17:3941038 | 77406 | 17:3948766 | 77407 | 17:3948856 | 77408 | 17:3950488 |
| 77409 | 17:3950503 | 77410 | 17:3979701 | 77413 | 17:4046782 | 77419 | 17:4192418 | 77424 | 17:4272755 |
| 77426 | 17:4298012 | 77432 | 17:4394112 | 77442 | 17:4571004 | 77447 | 17:4633530 | 77448 | 17:4657147 |
| 77453 | 17:4721455 | 77454 | 17:4721515 | 77456 | 17:4770586 | 77460 | 17:4835673 | 77465 | 17:4973219 |
| 77479 | 17:5317539 | 77481 | 17:5375833 | 77482 | 17:5398061 | 77490 | 17:5679415 | 77491 | 17:5679423 |
| 77492 | 17:5679424 | 77493 | 17:5679464 | 77494 | 17:5679532 | 77496 | 17:5728456 | 77512 | 17:6078923 |
| 77513 | 17:6107701 | 77516 | 17:6161274 | 77517 | 17:6198963 | 77518 | 17:6205937 | 77520 | 17:6220260 |
| 77521 | 17:6229560 | 77522 | 17:6258944 | 77526 | 17:6341387 | 77532 | 17:6459287 | 77533 | 17:6491029 |
| 77537 | 17:6552131 | 77539 | 17:6614404 | 77540 | 17:6625470 | 77541 | 17:6626902 | 77544 | 17:6698968 |
| 77545 | 17:6701688 | 77546 | 17:6707615 | 77547 | 17:6710298 | 77548 | 17:6715516 | 77554 | 17:6811535 |
| 77555 | 17:6819346 | 77559 | 17:6940926 | 77560 | 17:6984368 | 77561 | 17:6985910 | 77563 | 17:7027194 |
| 77588 | 17:7507100 | 77596 | 17:7694458 | 77598 | 17:7697113 | 77602 | 17:7770098 | 77612 | 17:8012088 |
| 77613 | 17:8012177 | 77621 | 17:8235574 | 77637 | 17:8588779 | 77661 | 17:9138287 | 77674 | 17:9441496 |
| 77683 | 17:9768434 | 77684 | 17:9768476 | 77688 | 17:9888649 | 77695 | 17:9978114 | 77696 | 17:10024882 |
| 77698 | 17:10050562 | 77700 | 17:10077655 | 77702 | 17:10100298 | 77705 | 17:10110670 | 77709 | 17:10169816 |
| 77710 | 17:10176209 | 77717 | 17:10353846 | 77720 | 17:10489157 | 77730 | 17:10813567 | 77731 | 17:10814317 |
| 77749 | 17:11330319 | 77765 | 17:11778925 | 77771 | 17:11817119 | 77780 | 17:12090766 | 77797 | 17:12362004 |
| 77814 | 17:12766769 | 77823 | 17:12938879 | 77828 | 17:13174673 | 77832 | 17:13361331 | 77837 | 17:13490837 |
| 77838 | 17:13490864 | 77849 | 17:13820980 | 77850 | 17:13883525 | 77853 | 17:13941930 | 77865 | 17:14194666 |
| 77866 | 17:14208519 | 77867 | 17:14212322 | 77868 | 17:14214868 | 77870 | 17:14226993 | 77871 | 17:14233961 |
| 77874 | 17:14255518 | 77875 | 17:14257411 | 77876 | 17:14258646 | 77877 | 17:14265993 | 77878 | 17:14267278 |
| 77879 | 17:14268573 | 77885 | 17:14447098 | 77889 | 17:14526830 | 77892 | 17:14548325 | 77897 | 17:14641198 |
| 77912 | 17:14905289 | 77913 | 17:14921802 | 77920 | 17:15088817 | 77925 | 17:15159023 | 77929 | 17:15328788 |
| 77931 | 17:15328888 | 77937 | 17:15455174 | 77940 | 17:15527126 | 77948 | 17:15652225 | 77952 | 17:15705665 |
| 77959 | 17:15911943 | 77967 | 17:16144638 | 77970 | 17:16239054 | 77981 | 17:16560132 | 77983 | 17:16659063 |
| 77984 | 17:16684179 | 77989 | 17:16808806 | 77990 | 17:16833027 | 78001 | 17:17127455 | 78002 | 17:17127599 |
| 78004 | 17:17132830 | 78007 | 17:17224074 | 78021 | 17:17389673 | 78028 | 17:17475047 | 78029 | 17:17475831 |
| 78048 | 17:17963586 | 78050 | 17:17992691 | 78054 | 17:18028087 | 78057 | 17:18062413 | 78060 | 17:18161738 |
| 78064 | 17:18284618 | 78087 | 17:18873882 | 78092 | 17:18900848 | 78094 | 17:18961495 | 78102 | 17:19217997 |
| 78130 | 17:19581434 | 78135 | 17:19786181 | 78141 | 17:19961480 | 78142 | 17:19965877 | 78144 | 17:20018582 |
| 78149 | 17:20111214 | 78152 | 17:20217310 | 78157 | 17:20333021 | 78227 | 17:22641428 | 78231 | 17:22853179 |
| 78245 | 17:23369245 | 78266 | 17:24189137 | 78275 | 17:24523000 | 78285 | 17:24792731 | 78294 | 17:25041571 |
| 78312 | 17:25581271 | 78323 | 17:25977654 | 78324 | 17:25977693 | 78325 | 17:25977723 | 78326 | 17:25977762 |
| 78369 | 17:26883751 | 78370 | 17:26915514 | 78393 | 17:27503090 | 78394 | 17:27559967 | 78399 | 17:27653769 |
| 78408 | 17:27921810 | 78413 | 17:28059892 | 78427 | 17:28334342 | 78428 | 17:28334349 | 78439 | 17:28702389 |
| 78464 | 17:29480103 | 78469 | 17:29662529 | 78478 | 17:29993789 | 78489 | 17:30232347 | 78490 | 17:30304435 |
| 78497 | 17:30487913 | 78510 | 17:30674204 | 78511 | 17:30696794 | 78512 | 17:30709277 | 78516 | 17:30799626 |
| 78524 | 17:30996169 | 78544 | 17:31562261 | 78549 | 17:31735789 | 78551 | 17:31735957 | 78561 | 17:31998920 |
| 78562 | 17:32030137 | 78571 | 17:32394529 | 78572 | 17:32394568 | 78573 | 17:32403823 | 78574 | 17:32403842 |
| 78594 | 17:32777788 | 78599 | 17:32874912 | 78618 | 17:33384490 | 78641 | 17:33842930 | 78655 | 17:34228544 |
| 78665 | 17:34544058 | 78678 | 17:34902455 | 78685 | 17:35149936 | 78686 | 17:35186932 | 78694 | 17:35488740 |
| 78704 | 17:35746205 | 78706 | 17:35787842 | 78707 | 17:35799086 | 78709 | 17:35857580 | 78712 | 17:35940925 |
| 78719 | 17:36144107 | 78728 | 17:36520470 | 78737 | 17:36882303 | 78781 | 17:38220984 | 78810 | 17:39276967 |
| 78812 | 17:39326538 | 78837 | 17:39843444 | 78842 | 17:39882804 | 78852 | 17:39935546 | 78860 | 17:40113609 |
| 78865 | 17:40279234 | 78867 | 17:40310128 | 78868 | 17:40314686 | 78879 | 17:40566975 | 78883 | 17:40575300 |
| 78886 | 17:40597293 | 78894 | 17:40765558 | 78897 | 17:40880376 | 78898 | 17:40882503 | 78905 | 17:41002973 |
| 78932 | 17:41864826 | 78941 | 17:42170894 | 78942 | 17:42186823 | 78943 | 17:42188310 | 78946 | 17:42229273 |
| 78954 | 17:42393090 | 78956 | 17:42415227 | 78970 | 17:42715046 | 78976 | 17:42768963 | 78978 | 17:42821182 |
| 78982 | 17:42891704 | 78983 | 17:42923614 | 78997 | 17:43357874 | 79000 | 17:43436343 | 79003 | 17:43553874 |
| 79006 | 17:43577102 | 79007 | 17:43577588 | 79008 | 17:43594706 | 79011 | 17:43609022 | 79012 | 17:43623722 |
| 79029 | 17:44194890 | 79032 | 17:44224761 | 79040 | 17:44531668 | 79046 | 17:44677660 | 79052 | 17:44819760 |
| 79058 | 17:44927880 | 79063 | 17:45086949 | 79064 | 17:45091135 | 79066 | 17:45172949 | 79068 | 17:45178502 |
| 79069 | 17:45178540 | 79070 | 17:45180519 | 79071 | 17:45180612 | 79072 | 17:45181370 | 79073 | 17:45182722 |
| 79091 | 17:45616057 | 79094 | 17:45734727 | 79112 | 17:46168639 | 79113 | 17:46171496 | 79127 | 17:46408391 |
| 79135 | 17:46615898 | 79136 | 17:46624459 | 79142 | 17:46676910 | 79187 | 17:47856534 | 79215 | 17:48637182 |
| 79244 | 17:49321412 | 79268 | 17:49848060 | 79273 | 17:49923094 | 79285 | 17:50262075 | 79287 | 17:50270777 |
| 79288 | 17:50274476 | 79315 | 17:50731825 | 79316 | 17:50769132 | 79319 | 17:50841533 | 79320 | 17:50864647 |
| 79322 | 17:50902320 | 79326 | 17:50955494 | 79327 | 17:50959261 | 79339 | 17:51345327 | 79342 | 17:51469617 |
| 79343 | 17:51470914 | 79346 | 17:51529156 | 79348 | 17:51546917 | 79355 | 17:51627470 | 79359 | 17:51685973 |
| 79360 | 17:51689778 | 79364 | 17:51762211 | 79366 | 17:51792442 | 79368 | 17:51813934 | 79375 | 17:51958814 |
| 79377 | 17:51959782 | 79379 | 17:52027381 | 79395 | 17:52307367 | 79403 | 17:52573801 | 79414 | 17:52988658 |
| 79417 | 17:53020835 | 79423 | 17:53275351 | 79428 | 17:53388401 | 79430 | 17:53390888 | 79431 | 17:53448341 |
| 79433 | 17:53470903 | 79435 | 17:53532750 | 79447 | 17:53919113 | 79454 | 17:54025669 | 79455 | 17:54029457 |
| 79456 | 17:54030902 | 79457 | 17:54033380 | 79458 | 17:54062683 | 79459 | 17:54122851 | 79461 | 17:54128175 |
| 79465 | 17:54163192 | 79468 | 17:54264980 | 79469 | 17:54267012 | 79470 | 17:54310765 | 79483 | 17:54668554 |
| 79489 | 17:54807887 | 79499 | 17:54895464 | 79505 | 17:55074569 | 79506 | 17:55081286 | 79507 | 17:55082228 |
| 79508 | 17:55105646 | 79509 | 17:55105686 | 79512 | 17:55385614 | 79517 | 17:55484565 | 79521 | 17:55592718 |
| 79523 | 17:55665541 | 79524 | 17:55716549 | 79526 | 17:55752066 | 79532 | 17:55876873 | 79537 | 17:56011560 |
| 79548 | 17:56279452 | 79549 | 17:56281095 | 79560 | 17:56566250 | 79564 | 17:56651078 | 79565 | 17:56710101 |
| 79571 | 17:56976866 | 79576 | 17:57116303 | 79583 | 17:57285859 | 79591 | 17:57472146 | 79598 | 17:57643506 |
| 79601 | 17:57716356 | 79602 | 17:57730355 | 79603 | 17:57731200 | 79610 | 17:57797646 | 79612 | 17:57831569 |
| 79623 | 17:58093779 | 79625 | 17:58112817 | 79626 | 17:58130419 | 79627 | 17:58141075 | 79634 | 17:58304066 |
| 79665 | 17:58556801 | 79670 | 17:58775371 | 79679 | 17:59008052 | 79688 | 17:59157020 | 79690 | 17:59215735 |
| 79700 | 17:59384904 | 79702 | 17:59397574 | 79710 | 17:59604994 | 79714 | 17:59716548 | 79719 | 17:59839482 |
| 79730 | 17:60067619 | 79731 | 17:60090877 | 79732 | 17:60090928 | 79733 | 17:60097662 | 79743 | 17:60248908 |
| 79751 | 17:60373041 | 79752 | 17:60374859 | 79753 | 17:60395890 | 79754 | 17:60395994 | 79760 | 17:60500390 |
| 79771 | 17:60710033 | 79772 | 17:60710035 | 79773 | 17:60710040 | 79774 | 17:60710101 | 79779 | 17:60795229 |
| 79780 | 17:60795274 | 79781 | 17:60795346 | 79784 | 17:60843399 | 79807 | 17:61141160 | 79812 | 17:61202979 |
| 79814 | 17:61208274 | 79815 | 17:61208429 | 79816 | 17:61237780 | 79819 | 17:61316287 | 79822 | 17:61370873 |
| 79828 | 17:61499579 | 79829 | 17:61499616 | 79842 | 17:61892683 | 79846 | 17:61971343 | 79850 | 17:62029219 |
| 79858 | 17:62310603 | 79866 | 17:62556427 | 79874 | 17:62749389 | 79882 | 17:62937635 | 79884 | 17:62967008 |
| 79886 | 17:63060456 | 79890 | 17:63193363 | 79904 | 17:63343949 | 79909 | 17:63512971 | 79912 | 17:63562509 |
| 79916 | 17:63612425 | 79917 | 17:63613044 | 79918 | 17:63614883 | 79919 | 17:63615321 | 79920 | 17:63615338 |
| 79921 | 17:63619368 | 79922 | 17:63619438 | 79923 | 17:63619955 | 79926 | 17:63623292 | 79927 | 17:63624426 |
| 79933 | 17:63696661 | 79937 | 17:63745078 | 79938 | 17:63765593 | 79946 | 17:63909068 | 79947 | 17:63911823 |
| 79948 | 17:63914506 | 79949 | 17:63924591 | 79953 | 17:64073618 | 79954 | 17:64077337 | 79955 | 17:64079380 |
| 79957 | 17:64164363 | 79959 | 17:64233930 | 79963 | 17:64320209 | 79964 | 17:64364133 | 79965 | 17:64376618 |
| 79966 | 17:64381714 | 79967 | 17:64390370 | 79968 | 17:64391421 | 79969 | 17:64392375 | 79971 | 17:64400070 |
| 79975 | 17:64543414 | 79983 | 17:64868669 | 79986 | 17:64936914 | 80004 | 17:65250897 | 80005 | 17:65252978 |
| 80007 | 17:65269001 | 80009 | 17:65311178 | 80015 | 17:65495113 | 80016 | 17:65515359 | 80028 | 17:65711335 |
| 80046 | 17:66084982 | 80048 | 17:66098132 | 80091 | 17:66926217 | 80093 | 17:66962321 | 80094 | 17:66963375 |
| 80095 | 17:66964444 | 80096 | 17:66980228 | 80097 | 17:66981196 | 80098 | 17:66983892 | 80099 | 17:66987390 |
| 80100 | 17:66989033 | 80101 | 17:66991682 | 80102 | 17:66993583 | 80117 | 17:67452220 | 80120 | 17:67493854 |
| 80123 | 17:67576683 | 80133 | 17:67991825 | 80143 | 17:68190606 | 80145 | 17:68235847 | 80146 | 17:68237141 |
| 80152 | 17:68336966 | 80154 | 17:68390649 | 80156 | 17:68404948 | 80165 | 17:68610408 | 80169 | 17:68711761 |
| 80177 | 17:68924124 | 80186 | 17:69025532 | 80187 | 17:69025781 | 80191 | 17:69104813 | 80200 | 17:69235524 |
| 80201 | 17:69244183 | 80208 | 17:69526999 | 80209 | 17:69557728 | 80210 | 17:69558386 | 80221 | 17:69723039 |
| 80226 | 17:69823676 | 80236 | 17:70089184 | 80242 | 17:70183712 | 80266 | 17:70611985 | 80267 | 17:70615923 |
| 80268 | 17:70617839 | 80269 | 17:70618022 | 80270 | 17:70618023 | 80271 | 17:70619770 | 80272 | 17:70621442 |
| 80294 | 17:71216081 | 80295 | 17:71224018 | 80297 | 17:71275992 | 80298 | 17:71276089 | 80299 | 17:71276886 |
| 80300 | 17:71276966 | 80301 | 17:71283804 | 80306 | 17:71441658 | 80309 | 17:71512874 | 80310 | 17:71518391 |
| 80311 | 17:71528096 | 80312 | 17:71528119 | 80313 | 17:71528120 | 80314 | 17:71528861 | 80315 | 17:71528882 |
| 80316 | 17:71528942 | 80324 | 17:71695918 | 80325 | 17:71702829 | 80328 | 17:71812939 | 80339 | 17:71951302 |
| 80349 | 17:72204450 | 80356 | 17:72342109 | 80358 | 17:72363855 | 80360 | 17:72369402 | 80362 | 17:72398155 |
| 80364 | 17:72408383 | 80365 | 17:72445427 | 80366 | 17:72447709 | 80367 | 17:72457694 | 80369 | 17:72468245 |
| 80370 | 17:72498960 | 80374 | 17:72555058 | 80384 | 17:72883784 | 80386 | 17:72916533 | 80391 | 17:73036168 |
| 80411 | 18:868580 | 80413 | 18:911112 | 80414 | 18:911196 | 80415 | 18:932024 | 80416 | 18:932036 |
| 80417 | 18:932132 | 80431 | 18:1178922 | 80435 | 18:1215919 | 80436 | 18:1269485 | 80437 | 18:1269511 |
| 80438 | 18:1269570 | 80452 | 18:1553979 | 80453 | 18:1565391 | 80460 | 18:1658820 | 80467 | 18:1733905 |
| 80468 | 18:1734779 | 80469 | 18:1737507 | 80470 | 18:1739009 | 80471 | 18:1741139 | 80472 | 18:1742546 |
| 80474 | 18:1743718 | 80475 | 18:1744700 | 80476 | 18:1745880 | 80477 | 18:1746570 | 80478 | 18:1747824 |
| 80479 | 18:1749671 | 80480 | 18:1751021 | 80481 | 18:1752037 | 80482 | 18:1753160 | 80483 | 18:1754297 |
| 80484 | 18:1754989 | 80485 | 18:1755466 | 80486 | 18:1758653 | 80487 | 18:1759177 | 80488 | 18:1759407 |
| 80489 | 18:1759775 | 80490 | 18:1760828 | 80491 | 18:1761351 | 80492 | 18:1761856 | 80493 | 18:1764061 |
| 80494 | 18:1765063 | 80495 | 18:1765811 | 80496 | 18:1766413 | 80497 | 18:1766979 | 80498 | 18:1780698 |
| 80499 | 18:1799373 | 80503 | 18:1821333 | 80515 | 18:2132264 | 80519 | 18:2189010 | 80525 | 18:2310539 |
| 80529 | 18:2407203 | 80532 | 18:2547395 | 80535 | 18:2627721 | 80548 | 18:2801078 | 80549 | 18:2824948 |
| 80553 | 18:2893572 | 80559 | 18:2948533 | 80590 | 18:3732736 | 80593 | 18:3803407 | 80599 | 18:3918400 |
| 80604 | 18:4117718 | 80610 | 18:4175893 | 80622 | 18:4456924 | 80623 | 18:4458325 | 80652 | 18:5105107 |
| 80657 | 18:5261361 | 80658 | 18:5303405 | 80667 | 18:5381034 | 80668 | 18:5392289 | 80686 | 18:5796418 |
| 80695 | 18:6075338 | 80699 | 18:6140925 | 80700 | 18:6150654 | 80702 | 18:6155426 | 80708 | 18:6179950 |
| 80710 | 18:6195750 | 80717 | 18:6307757 | 80732 | 18:6411938 | 80733 | 18:6414217 | 80734 | 18:6434042 |
| 80736 | 18:6473072 | 80742 | 18:6606486 | 80756 | 18:6904961 | 80759 | 18:6952523 | 80775 | 18:7393588 |
| 80782 | 18:7571670 | 80783 | 18:7571744 | 80784 | 18:7571766 | 80790 | 18:7722433 | 80791 | 18:7725494 |
| 80792 | 18:7759211 | 80794 | 18:7785017 | 80795 | 18:7806089 | 80798 | 18:7857634 | 80809 | 18:8151586 |
| 80814 | 18:8174600 | 80816 | 18:8217290 | 80817 | 18:8219384 | 80818 | 18:8219385 | 80819 | 18:8219497 |
| 80836 | 18:8556730 | 80847 | 18:8895607 | 80850 | 18:8930714 | 80868 | 18:9094422 | 80869 | 18:9121273 |
| 80871 | 18:9155492 | 80873 | 18:9160707 | 80877 | 18:9224812 | 80879 | 18:9281528 | 80891 | 18:9500208 |
| 80893 | 18:9514100 | 80897 | 18:9541606 | 80898 | 18:9560416 | 80903 | 18:9622228 | 80913 | 18:9939597 |
| 80915 | 18:9939685 | 80920 | 18:10053603 | 80937 | 18:10466956 | 80938 | 18:10467016 | 80939 | 18:10474781 |
| 80940 | 18:10474891 | 80944 | 18:10554759 | 80945 | 18:10555268 | 80949 | 18:10623982 | 80954 | 18:10638844 |
| 80955 | 18:10661516 | 80959 | 18:10775715 | 80966 | 18:10924943 | 80970 | 18:11032803 | 80972 | 18:11076163 |
| 80975 | 18:11143812 | 80978 | 18:11177661 | 80979 | 18:11189421 | 80982 | 18:11197228 | 80985 | 18:11315060 |
| 80986 | 18:11322791 | 80987 | 18:11333644 | 80988 | 18:11340350 | 80989 | 18:11349111 | 80990 | 18:11352571 |
| 80991 | 18:11362283 | 80992 | 18:11367578 | 80993 | 18:11398671 | 80995 | 18:11499305 | 80996 | 18:11499360 |
| 80997 | 18:11589376 | 80999 | 18:11631258 | 81001 | 18:11639177 | 81002 | 18:11640852 | 81004 | 18:11652518 |
| 81007 | 18:11719208 | 81026 | 18:12374145 | 81030 | 18:12487243 | 81036 | 18:12677943 | 81058 | 18:13316111 |
| 81059 | 18:13322081 | 81060 | 18:13326301 | 81068 | 18:13483552 | 81090 | 18:14035593 | 81092 | 18:14117250 |
| 81097 | 18:14266021 | 81104 | 18:14579399 | 81110 | 18:14880493 | 81111 | 18:14918649 | 81121 | 18:15157858 |
| 81129 | 18:15352349 | 81130 | 18:15353723 | 81136 | 18:15529204 | 81137 | 18:15532676 | 81138 | 18:15535125 |
| 81139 | 18:15537825 | 81142 | 18:15574419 | 81145 | 18:15607698 | 81147 | 18:15643821 | 81149 | 18:15771762 |
| 81163 | 18:16246520 | 81164 | 18:16246606 | 81165 | 18:16246649 | 81167 | 18:16294087 | 81170 | 18:16339724 |
| 81179 | 18:16491063 | 81186 | 18:16701247 | 81188 | 18:16722963 | 81191 | 18:16855403 | 81194 | 18:16929518 |
| 81198 | 18:16960682 | 81205 | 18:17095383 | 31206 | 18:17095391 | 81207 | 18:17095421 | 81208 | 18:17128188 |
| 81214 | 18:17231296 | 81219 | 18:17332696 | 81224 | 18:17474883 | 81225 | 18:17474962 | 81246 | 18:17964994 |
| 81248 | 18:18015282 | 81259 | 18:18234827 | 31274 | 18:18340451 | 81277 | 18:18455809 | 81281 | 18:18555382 |
| 81286 | 18:18634395 | 81288 | 18:18652008 | 81292 | 18:18693621 | 81296 | 18:18741396 | 81297 | 18:18766595 |
| 81304 | 18:18991234 | 81308 | 18:19028089 | 81312 | 18:19104084 | 81318 | 18:19279732 | 81319 | 18:19306933 |
| 81321 | 18:19321180 | 81328 | 18:19549103 | 81337 | 18:19828522 | 81341 | 18:19884211 | 81346 | 18:19992495 |
| 81379 | 18:20783780 | 81396 | 18:21231861 | 81397 | 18:21231877 | 81404 | 18:21429864 | 81405 | 18:21429866 |
| 81419 | 18:21805702 | 81422 | 18:21842921 | 81427 | 18:21915772 | 81428 | 18:21944535 | 81429 | 18:21944650 |
| 81439 | 18:22075634 | 81459 | 18:22432351 | 81467 | 18:22574217 | 81468 | 18:22581424 | 81470 | 18:22616518 |
| 81489 | 18:23048862 | 81515 | 18:23639968 | 81523 | 18:23885882 | 81526 | 18:23978279 | 81530 | 18:24024754 |
| 81531 | 18:24028549 | 81532 | 18:24039248 | 81538 | 18:24159516 | 81541 | 18:24233256 | 81547 | 18:24449226 |
| 81548 | 18:24467752 | 81551 | 18:24569333 | 81555 | 18:24603788 | 81556 | 18:24605526 | 81557 | 18:24606504 |
| 81558 | 18:24609637 | 81559 | 18:24622318 | 81560 | 18:24626076 | 81561 | 18:24627052 | 81562 | 18:24640038 |
| 81563 | 18:24646900 | 81564 | 18:24648311 | 81565 | 18:24650296 | 81566 | 18:24653596 | 81567 | 18:24655887 |
| 81568 | 18:24658898 | 81569 | 18:24680546 | 81572 | 18:24703105 | 81573 | 18:24711358 | 81581 | 18:24916840 |
| 81586 | 18:25087427 | 81594 | 18:25243259 | 81595 | 18:25263618 | 81597 | 18:25320467 | 81603 | 18:25475882 |
| 81604 | 18:25507140 | 81608 | 18:25560734 | 81613 | 18:25671777 | 81614 | 18:25693559 | 81617 | 18:25769838 |
| 81618 | 18:25772348 | 81619 | 18:25777563 | 81622 | 18:25833720 | 81626 | 18:25944954 | 81638 | 18:26118698 |
| 81643 | 18:26252156 | 81648 | 18:26367037 | 81652 | 18:26448739 | 81674 | 18:26911688 | 81683 | 18:27123603 |
| 81711 | 18:27938774 | 81712 | 18:27938830 | 81733 | 18:28523029 | 81752 | 18:29298380 | 81753 | 18:29324758 |
| 81755 | 18:29354497 | 81756 | 18:29354560 | 81757 | 18:29354692 | 81758 | 18:29356162 | 81759 | 18:29359726 |
| 81760 | 18:29359801 | 81765 | 18:29507594 | 81766 | 18:29507596 | 81767 | 18:29507695 | 81768 | 18:29517248 |
| 81800 | 18:30444281 | 81812 | 18:30824413 | 81820 | 18:31114666 | 81821 | 18:31161073 | 81829 | 18:31403926 |
| 81860 | 18:32193066 | 81865 | 18:32277972 | 81866 | 18:32277986 | 81889 | 18:32769354 | 81892 | 18:32797023 |
| 81893 | 18:32809799 | 81909 | 18:33174459 | 81913 | 18:33266043 | 81915 | 18:33332355 | 81928 | 18:33524126 |
| 81938 | 18:33726109 | 81942 | 18:33803131 | 81943 | 18:33819518 | 81948 | 18:33891121 | 81950 | 18:33985539 |
| 81958 | 18:34273373 | 81960 | 18:34305936 | 81962 | 18:34318674 | 81963 | 18:34340817 | 81967 | 18:34415049 |
| 81968 | 18:34460275 | 81969 | 18:34460342 | 81971 | 18:34477636 | 81973 | 18:34507746 | 81976 | 18:34573297 |
| 81979 | 18:34674118 | 81981 | 18:34742637 | 81985 | 18:34868221 | 81993 | 18:35213610 | 81994 | 18:35247365 |
| 81996 | 18:35258480 | 82001 | 18:35371180 | 82003 | 18:35406541 | 82008 | 18:35505165 | 82009 | 18:35515593 |
| 82010 | 18:35575292 | 82012 | 18:35602009 | 82016 | 18:35713513 | 82021 | 18:35869059 | 82027 | 18:35949415 |
| 82029 | 18:35961563 | 82035 | 18:36058481 | 82036 | 18:36063927 | 82038 | 18:36087372 | 82039 | 18:36087440 |
| 82040 | 18:36093659 | 82042 | 18:36121381 | 82046 | 18:36318653 | 82054 | 18:36585013 | 82059 | 18:36712932 |
| 82066 | 18:36883800 | 82067 | 18:36890174 | 82069 | 18:36892550 | 82093 | 18:37455502 | 82098 | 18:37533228 |
| 82102 | 18:37619375 | 82122 | 18:38001454 | 82131 | 18:38196771 | 82133 | 18:38218416 | 82135 | 18:38277631 |
| 82138 | 18:38301449 | 82141 | 18:38331320 | 82150 | 18:38614192 | 82174 | 18:39233646 | 82175 | 18:39286312 |
| 82180 | 18:39414004 | 82182 | 18:39447432 | 82189 | 18:39589574 | 82192 | 18:39612914 | 82215 | 18:40137391 |
| 82216 | 18:40137453 | 82217 | 18:40138746 | 82218 | 18:40138750 | 82219 | 18:40138985 | 82220 | 18:40139048 |
| 82222 | 18:40176507 | 82223 | 18:40176673 | 82228 | 18:40316884 | 82230 | 18:40330132 | 82237 | 18:40533358 |
| 82238 | 18:40534569 | 82241 | 18:40597479 | 82249 | 18:40763764 | 82250 | 18:40763815 | 82254 | 18:40819026 |
| 82255 | 18:40819036 | 82261 | 18:40956148 | 82269 | 18:41180952 | 82274 | 18:41372533 | 82275 | 18:41391080 |
| 82277 | 18:41412597 | 82304 | 18:42002651 | 82308 | 18:42064307 | 82314 | 18:42154655 | 82325 | 18:42312455 |
| 82342 | 18:42673603 | 82352 | 18:42839761 | 82355 | 18:42870031 | 82356 | 18:42870306 | 82357 | 18:42870636 |
| 82373 | 18:43209321 | 82378 | 18:43300251 | 82397 | 18:43597221 | 82399 | 18:43723768 | 82400 | 18:43729208 |
| 82401 | 18:43744525 | 82403 | 18:43765343 | 82415 | 18:44100679 | 82418 | 18:44219776 | 82433 | 18:44678130 |
| 82437 | 18:44746464 | 82440 | 18:44842043 | 82444 | 18:44999448 | 82448 | 18:45230454 | 82451 | 18:45235853 |
| 82453 | 18:45282086 | 82454 | 18:45368501 | 82457 | 18:45512461 | 82459 | 18:45555708 | 82460 | 18:45555738 |
| 82461 | 18:45555791 | 82463 | 18:45568910 | 82474 | 18:45823041 | 82477 | 18:45919734 | 82481 | 18:46058902 |
| 82482 | 18:46058908 | 82483 | 18:46059262 | 82484 | 18:46059448 | 82485 | 18:46059625 | 82486 | 18:46074456 |
| 82488 | 18:46098110 | 82491 | 18:46151119 | 82493 | 18:46204752 | 82500 | 18:46339447 | 82506 | 18:46411478 |
| 82507 | 18:46412270 | 82510 | 18:46452569 | 82511 | 18:46454259 | 82514 | 18:46462113 | 82515 | 18:46472984 |
| 82516 | 18:46474223 | 82517 | 18:46474231 | 82518 | 18:46474239 | 82519 | 18:46474366 | 82520 | 18:46480475 |
| 82521 | 18:46481003 | 82522 | 18:46481046 | 82523 | 18:46481053 | 82524 | 18:46481480 | 82525 | 18:46481499 |
| 82526 | 18:46489662 | 82527 | 18:46490303 | 82528 | 18:46490881 | 82529 | 18:46498795 | 82530 | 18:46499716 |
| 82531 | 18:46500155 | 82532 | 18:46511353 | 82533 | 18:46512103 | 82534 | 18:46512104 | 82535 | 18:46523855 |
| 82536 | 18:46528787 | 82540 | 18:46627346 | 82542 | 18:46696595 | 82543 | 18:46697798 | 82544 | 18:46727043 |
| 82546 | 18:46727075 | 82547 | 18:46731490 | 82548 | 18:46731757 | 82549 | 18:46731785 | 82550 | 18:46731861 |
| 82551 | 18:46742887 | 82552 | 18:46742932 | 82553 | 18:46742940 | 82554 | 18:46743003 | 82555 | 18:46745292 |
| 82556 | 18:46745318 | 82557 | 18:46745397 | 82558 | 18:46745402 | 82559 | 18:46747438 | 82560 | 18:46747523 |
| 82561 | 18:46751198 | 82562 | 18:46751982 | 82563 | 18:46751987 | 82564 | 18:46752090 | 82565 | 18:46754516 |
| 82567 | 18:46762245 | 82568 | 18:46762248 | 82569 | 18:46763772 | 82570 | 18:46764309 | 82571 | 18:46764530 |
| 82572 | 18:46764820 | 82573 | 18:46764937 | 82574 | 18:46765974 | 82575 | 18:46766007 | 82576 | 18:46766010 |
| 82577 | 18:46771417 | 82578 | 18:46772451 | 82581 | 18:46793532 | 82585 | 18:46926291 | 82586 | 18:46926325 |
| 82587 | 18:46935389 | 82588 | 18:46936632 | 82589 | 18:46936776 | 82592 | 18:46961882 | 82593 | 18:46987640 |
| 82606 | 18:47331147 | 82607 | 18:47350791 | 82609 | 18:47380300 | 82614 | 18:47561882 | 82615 | 18:47569792 |
| 82616 | 18:47584273 | 82623 | 18:47724055 | 82630 | 18:47880052 | 82632 | 18:47904454 | 82633 | 18:47905402 |
| 82635 | 18:47914575 | 82638 | 18:47982264 | 82639 | 18:47986634 | 82645 | 18:48080577 | 82646 | 18:48082036 |
| 82647 | 18:48092307 | 82648 | 18:48093267 | 82649 | 18:48094554 | 82650 | 18:48095808 | 82651 | 18:48097168 |
| 82652 | 18:48098873 | 82653 | 18:48099751 | 82654 | 18:48100359 | 82655 | 18:48102403 | 82656 | 18:48103383 |
| 82657 | 18:48105090 | 82658 | 18:48105806 | 82660 | 18:48115016 | 82661 | 18:48119818 | 82662 | 18:48164016 |
| 82669 | 18:48328458 | 82674 | 18:48486626 | 82677 | 18:48540681 | 82678 | 18:48540711 | 82681 | 18:48550284 |
| 82682 | 18:48552506 | 82684 | 18:48648276 | 82685 | 18:48661989 | 82692 | 18:48824317 | 82700 | 18:49125312 |
| 82709 | 18:49431844 | 82710 | 18:49440743 | 82718 | 18:49685722 | 82720 | 18:49690710 | 82722 | 18:49774209 |
| 82727 | 18:49905675 | 82734 | 18:49974216 | 82744 | 18:50307933 | 82768 | 18:50909576 | 82776 | 18:51192874 |
| 82780 | 18:51273762 | 82784 | 18:51393314 | 82799 | 18:51647623 | 82801 | 18:51684659 | 82803 | 18:51687197 |
| 82804 | 18:51691345 | 82805 | 18:51691402 | 82806 | 18:51691426 | 82807 | 18:51691907 | 82808 | 18:51692088 |
| 82810 | 18:51703006 | 82811 | 18:51703434 | 82813 | 18:51710422 | 82820 | 18:51868158 | 82821 | 18:51868247 |
| 82822 | 18:51877833 | 82823 | 18:51888735 | 82826 | 18:51959716 | 82833 | 18:52170747 | 82843 | 18:52408540 |
| 82844 | 18:52434788 | 82846 | 18:52463921 | 82848 | 18:52526615 | 82849 | 18:52569137 | 82850 | 18:52586631 |
| 82856 | 18:52750690 | 82863 | 18:53056273 | 82864 | 18:53066684 | 82876 | 18:53447947 | 82877 | 18:53462716 |
| 82878 | 18:53515653 | 82880 | 18:53545259 | 82883 | 18:53565634 | 82885 | 18:53596284 | 82889 | 18:53631016 |
| 82890 | 18:53635483 | 82891 | 18:53666625 | 82892 | 18:53680050 | 82902 | 18:53873089 | 82903 | 18:53882150 |
| 82907 | 18:53924797 | 82908 | 18:53926298 | 82909 | 18:53938427 | 82910 | 18:53965820 | 82911 | 18:53970987 |
| 82912 | 18:53987026 | 82913 | 18:53988178 | 82917 | 18:54051234 | 82922 | 18:54129853 | 82924 | 18:54256059 |
| 82929 | 18:54326329 | 82930 | 18:54348038 | 82931 | 18:54348158 | 82932 | 18:54356059 | 82943 | 18:54594314 |
| 82945 | 18:54606774 | 82946 | 18:54607245 | 82947 | 18:54615284 | 82948 | 18:54615550 | 82949 | 18:54618878 |
| 82951 | 18:54632037 | 82952 | 18:54674333 | 82956 | 18:54725999 | 82957 | 18:54726122 | 82958 | 18:54732855 |
| 82965 | 18:54920625 | 82978 | 18:55105119 | 82980 | 18:55125421 | 82981 | 18:55130089 | 82991 | 18:55397342 |
| 82993 | 18:55448102 | 82994 | 18:55479044 | 83003 | 18:55722088 | 83006 | 18:55855289 | 83010 | 18:55908909 |
| 83022 | 18:56235646 | 83025 | 18:56256914 | 83027 | 18:56286701 | 83028 | 18:56286760 | 83030 | 18:56311848 |
| 83037 | 18:56458961 | 83039 | 18:56480496 | 83040 | 18:56481635 | 83041 | 18:56500189 | 83043 | 18:56516815 |
| 83046 | 18:56550331 | 83051 | 18:56657542 | 83053 | 18:56669005 | 83054 | 18:56669128 | 83055 | 18:56679475 |
| 83114 | 18:57068537 | 83115 | 18:57072585 | 83119 | 18:57151314 | 83120 | 18:57195331 | 83126 | 18:57435685 |
| 83134 | 18:57498462 | 83140 | 18:57652070 | 83141 | 18:57658393 | 83142 | 18:57662151 | 83143 | 18:57673429 |
| 83149 | 18:57750585 | 83150 | 18:57764229 | 83151 | 18:57774781 | 83159 | 18:57875057 | 83160 | 18:57880006 |
| 83162 | 18:57922196 | 83163 | 18:57922208 | 83168 | 18:57950000 | 83184 | 18:58213698 | 83185 | 18:58215105 |
| 83186 | 18:58221738 | 83187 | 18:58237470 | 83189 | 18:58270823 | 83190 | 18:58280185 | 83191 | 18:58285064 |
| 83192 | 18:58286528 | 83193 | 18:58288374 | 83194 | 18:58289386 | 83195 | 18:58290105 | 83196 | 18:58292173 |
| 83197 | 18:58292702 | 83198 | 18:58293461 | 83199 | 18:58294515 | 83200 | 18:58296803 | 83201 | 18:58297833 |
| 83202 | 18:58298676 | 83203 | 18:58302578 | 83204 | 18:58303580 | 83205 | 18:58304589 | 83206 | 18:58306121 |
| 83207 | 18:58308817 | 83208 | 18:58309408 | 83209 | 18:58311748 | 83210 | 18:58314758 | 83211 | 18:58319283 |
| 83213 | 18:58328404 | 83214 | 18:58335688 | 83215 | 18:58336548 | 83217 | 18:58339398 | 83218 | 18:58340297 |
| 83219 | 18:58341452 | 83220 | 18:58343184 | 83221 | 18:58346217 | 83222 | 18:58359203 | 83223 | 18:58360688 |
| 83224 | 18:58362462 | 83225 | 18:58369410 | 83226 | 18:58372976 | 83228 | 18:58501449 | 83259 | 18:59941034 |
| 83264 | 18:60057741 | 83276 | 18:60557876 | 83284 | 18:60892284 | 83285 | 18:60892325 | 83286 | 18:60892345 |
| 83287 | 18:60908498 | 83296 | 18:61230100 | 83297 | 18:61248798 | 83306 | 18:61528542 | 83314 | 18:61669548 |
| 83316 | 18:61688550 | 83327 | 18:61924215 | 83328 | 18:61925438 | 83329 | 18:61925595 | 83331 | 18:61938092 |
| 83332 | 18:61941006 | 83333 | 18:61948334 | 83334 | 18:61949940 | 83336 | 18:61962954 | 83337 | 18:61968568 |
| 83338 | 18:61969417 | 83339 | 18:61975282 | 83340 | 18:61976235 | 83341 | 18:61977792 | 83343 | 18:61979272 |
| 83344 | 18:61986664 | 83345 | 18:61987641 | 83347 | 18:62017097 | 83348 | 18:62031220 | 83350 | 18:62050366 |
| 83351 | 18:62055500 | 83352 | 18:62069703 | 83355 | 18:62098858 | 83356 | 18:62119696 | 83365 | 18:62301361 |
| 83369 | 18:62396313 | 83370 | 18:62411556 | 83371 | 18:62412333 | 83372 | 18:62491921 | 83391 | 18:63189938 |
| 83392 | 18:63193118 | 83393 | 18:63203763 | 83396 | 18:63217724 | 83397 | 18:63217760 | 83398 | 18:63217761 |
| 83401 | 18:63282873 | 83406 | 18:63442841 | 83407 | 18:63443616 | 83410 | 18:63449366 | 83413 | 18:63478461 |
| 83420 | 18:63566906 | 83435 | 18:63851165 | 83438 | 18:63870217 | 83439 | 18:63870560 | 83440 | 18:63870634 |
| 83441 | 18:63878287 | 83446 | 18:64020285 | 83448 | 18:64103695 | 83449 | 18:64107247 | 83450 | 18:64118087 |
| 83459 | 18:64261304 | 83464 | 18:64351790 | 83473 | 18:64453963 | 83475 | 18:64457710 | 83480 | 18:64586875 |
| 83481 | 18:64586957 | 83483 | 18:64587029 | 83484 | 18:64588207 | 83485 | 18:64588277 | 83486 | 18:64594228 |
| 83489 | 18:64607295 | 83493 | 18:64684413 | 83500 | 18:64871544 | 83506 | 18:64979546 | 83507 | 18:64989257 |
| 83508 | 18:65000708 | 83512 | 18:65024355 | 83513 | 18:65052564 | 83514 | 18:65058054 | 83515 | 18:65058108 |
| 83516 | 18:65058135 | 83522 | 18:65143297 | 83538 | 18:65213339 | 83546 | 18:65269401 | 83570 | 18:65467804 |
| 83573 | 18:65499590 | 83576 | 18:65531536 | 83582 | 18:65680076 | 83584 | 18:65778621 | 83611 | 19:1044735 |
| 83641 | 19:2028992 | 83643 | 19:2087607 | 83647 | 19:2189142 | 83648 | 19:2189181 | 83669 | 19:3019557 |
| 83676 | 19:3224135 | 83684 | 19:3507467 | 83688 | 19:3693922 | 83689 | 19:3693998 | 83690 | 19:3694048 |
| 83691 | 19:3717004 | 83712 | 19:4248938 | 83719 | 19:4532769 | 83732 | 19:4885934 | 83737 | 19:5057366 |
| 83738 | 19:5077598 | 83742 | 19:5131889 | 83743 | 19:5144117 | 83748 | 19:5302256 | 83758 | 19:5544452 |
| 83759 | 19:5550410 | 83770 | 19:5841378 | 83773 | 19:5911200 | 83777 | 19:6008981 | 83779 | 19:6069254 |
| 83780 | 19:6080767 | 83802 | 19:6741959 | 83803 | 19:6742031 | 83804 | 19:6744112 | 83808 | 19:6821054 |
| 83810 | 19:6854445 | 83823 | 19:7084364 | 83825 | 19:7139360 | 83830 | 19:7250907 | 83835 | 19:7311199 |
| 83857 | 19:7610709 | 83860 | 19:7655487 | 83867 | 19:7877242 | 83870 | 19:7955627 | 83874 | 19:8012167 |
| 83882 | 19:8102634 | 83884 | 19:8134333 | 83888 | 19:8242768 | 83890 | 19:8251385 | 83891 | 19:8253685 |
| 83892 | 19:8253738 | 83893 | 19:8256106 | 83894 | 19:8257817 | 83896 | 19:8303146 | 83897 | 19:8307007 |
| 83899 | 19:8318345 | 83909 | 19:8375150 | 83914 | 19:8447018 | 83917 | 19:8481149 | 83919 | 19:8538010 |
| 83928 | 19:8786239 | 83937 | 19:8921388 | 83938 | 19:8940266 | 83939 | 19:8940272 | 83946 | 19:9148135 |
| 83948 | 19:9161538 | 83951 | 19:9186570 | 83952 | 19:9186624 | 83953 | 19:9186684 | 83954 | 19:9186696 |
| 83955 | 19:9186752 | 83960 | 19:9321464 | 83963 | 19:9325083 | 83964 | 19:9329176 | 83971 | 19:9494606 |
| 83973 | 19:9527082 | 83974 | 19:9549010 | 83976 | 19:9584099 | 83978 | 19:9638098 | 83996 | 19:10144335 |
| 84000 | 19:10161366 | 84001 | 19:10185318 | 84002 | 19:10193665 | 84004 | 19:10203916 | 84005 | 19:10212410 |
| 84006 | 19:10222141 | 84012 | 19:10311673 | 84015 | 19:10357642 | 84018 | 19:10410339 | 84027 | 19:10608070 |
| 84028 | 19:10663875 | 84033 | 19:10816146 | 84048 | 19:11195343 | 84049 | 19:11206085 | 84050 | 19:11223181 |
| 84051 | 19:11223607 | 84074 | 19:11794721 | 84081 | 19:12080713 | 84098 | 19:12520276 | 84106 | 19:12624098 |
| 84115 | 19:12851003 | 84118 | 19:12942065 | 84119 | 19:12942066 | 84138 | 19:13385076 | 84153 | 19:13539153 |
| 84154 | 19:13540416 | 84156 | 19:13546434 | 84157 | 19:13553916 | 84158 | 19:13554659 | 84160 | 19:13567678 |
| 84161 | 19:13567681 | 84162 | 19:13569519 | 84163 | 19:13589753 | 84167 | 19:13627880 | 84168 | 19:13629329 |
| 84182 | 19:13922585 | 84184 | 19:13992338 | 84185 | 19:13992353 | 84193 | 19:14098428 | 84197 | 19:14234372 |
| 84199 | 19:14285305 | 84202 | 19:14359808 | 84206 | 19:14455998 | 84207 | 19:14458140 | 84208 | 19:14460416 |
| 84209 | 19:14461216 | 84212 | 19:14524347 | 84215 | 19:14594291 | 84231 | 19:14987147 | 84233 | 19:14997330 |
| 84236 | 19:15044294 | 84238 | 19:15079297 | 84265 | 19:15653982 | 84275 | 19:15950588 | 84282 | 19:16153652 |
| 84285 | 19:16179422 | 84286 | 19:16179458 | 84290 | 19:16243199 | 84291 | 19:16245110 | 84294 | 19:16316143 |
| 84296 | 19:16414613 | 84297 | 19:16414653 | 84298 | 19:16414656 | 84299 | 19:16414688 | 84300 | 19:16414750 |
| 84301 | 19:16414769 | 84305 | 19:16538181 | 84306 | 19:16538222 | 84307 | 19:16538228 | 84308 | 19:16538286 |
| 84309 | 19:16538332 | 84314 | 19:16680352 | 84315 | 19:16698614 | 84316 | 19:16701167 | 84317 | 19:16704714 |
| 84318 | 19:16710947 | 84319 | 19:16712329 | 84320 | 19:16715359 | 84329 | 19:16900771 | 84344 | 19:17139083 |
| 84345 | 19:17193285 | 84347 | 19:17219724 | 84354 | 19:17346967 | 84358 | 19:17413575 | 84360 | 19:17439500 |
| 84361 | 19:17463604 | 84362 | 19:17463752 | 84363 | 19:17463766 | 84364 | 19:17463785 | 84366 | 19:17476802 |
| 84367 | 19:17485925 | 84381 | 19:17839100 | 84382 | 19:17840344 | 84383 | 19:17847133 | 84388 | 19:17935227 |
| 84390 | 19:18002956 | 84391 | 19:18012136 | 84405 | 19:18364670 | 84427 | 19:18812123 | 84429 | 19:18935333 |
| 84434 | 19:19027484 | 84438 | 19:19066522 | 84440 | 19:19164165 | 84441 | 19:19167861 | 84443 | 19:19248544 |
| 84448 | 19:19342984 | 84455 | 19:19497354 | 84456 | 19:19503339 | 84469 | 19:19832430 | 84470 | 19:19832910 |
| 84473 | 19:19891932 | 84478 | 19:20010169 | 84479 | 19:20010699 | 84480 | 19:20021077 | 84481 | 19:20024935 |
| 84482 | 19:20039821 | 84483 | 19:20039838 | 84484 | 19:20049560 | 84486 | 19:20064375 | 84487 | 19:20065239 |
| 84488 | 19:20065746 | 84489 | 19:20067184 | 84490 | 19:20067197 | 84496 | 19:20169459 | 84497 | 19:20252737 |
| 84498 | 19:20269891 | 84499 | 19:20272304 | 84500 | 19:20272993 | 84501 | 19:20272997 | 84502 | 19:20287832 |
| 84503 | 19:20347363 | 84504 | 19:20347364 | 84505 | 19:20347398 | 84506 | 19:20347408 | 84507 | 19:20347436 |
| 84508 | 19:20347583 | 84511 | 19:20397604 | 84513 | 19:20404265 | 84514 | 19:20406390 | 84515 | 19:20407245 |
| 84516 | 19:20407776 | 84517 | 19:20407895 | 84518 | 19:20408583 | 84519 | 19:20409859 | 84520 | 19:20410892 |
| 84521 | 19:20411434 | 84522 | 19:20412101 | 84523 | 19:20413435 | 84524 | 19:20414800 | 84525 | 19:20415545 |
| 84526 | 19:20415733 | 84527 | 19:20416065 | 84528 | 19:20416081 | 84529 | 19:20416676 | 84530 | 19:20417918 |
| 84531 | 19:20418417 | 84532 | 19:20418508 | 84533 | 19:20418613 | 84535 | 19:20421499 | 84536 | 19:20423129 |
| 84537 | 19:20427231 | 84538 | 19:20443801 | 84539 | 19:20448136 | 84540 | 19:20449515 | 84541 | 19:20450452 |
| 84542 | 19:20451130 | 84543 | 19:20454459 | 84544 | 19:20459782 | 84545 | 19:20464970 | 84546 | 19:20466789 |
| 84547 | 19:20468157 | 84548 | 19:20469610 | 84557 | 19:20885539 | 84561 | 19:20948913 | 84570 | 19:21194708 |
| 84571 | 19:21195341 | 84580 | 19:21541775 | 84582 | 19:21606050 | 84583 | 19:21606066 | 84584 | 19:21606170 |
| 84586 | 19:21607755 | 84587 | 19:21607870 | 84588 | 19:21657448 | 84596 | 19:21761419 | 84601 | 19:21810539 |
| 84602 | 19:21810883 | 84608 | 19:22007200 | 84613 | 19:22125908 | 84617 | 19:22209844 | 84618 | 19:22216194 |
| 84619 | 19:22216936 | 84620 | 19:22218946 | 84621 | 19:22223761 | 84622 | 19:22225302 | 84624 | 19:22228117 |
| 84625 | 19:22228881 | 84626 | 19:22238525 | 84629 | 19:22288784 | 84632 | 19:22387067 | 84637 | 19:22435985 |
| 84640 | 19:22580315 | 84642 | 19:22630921 | 84645 | 19:22686577 | 84649 | 19:22777911 | 84653 | 19:22919648 |
| 84654 | 19:22933829 | 84655 | 19:22937788 | 84659 | 19:23047864 | 84663 | 19:23089782 | 84665 | 19:23103859 |
| 84666 | 19:23125220 | 84677 | 19:23322846 | 84678 | 19:23328857 | 84691 | 19:23748456 | 84692 | 19:23762942 |
| 84696 | 19:23795890 | 84699 | 19:23844740 | 84709 | 19:24085976 | 84710 | 19:24086312 | 84711 | 19:24086389 |
| 84712 | 19:24100573 | 84713 | 19:24119941 | 84714 | 19:24120015 | 84715 | 19:24120089 | 84716 | 19:24120654 |
| 84717 | 19:24120726 | 84719 | 19:24158070 | 84720 | 19:24171676 | 84721 | 19:24190844 | 84722 | 19:24190846 |
| 84723 | 19:24190851 | 84724 | 19:24190893 | 84725 | 19:24201030 | 84726 | 19:24201096 | 84727 | 19:24203359 |
| 84728 | 19:24203402 | 84729 | 19:24203454 | 84730 | 19:24204657 | 84731 | 19:24206255 | 84732 | 19:24206362 |
| 84733 | 19:24210049 | 84734 | 19:24211193 | 84740 | 19:24352581 | 84749 | 19:24484569 | 84752 | 19:24575685 |
| 84757 | 19:24628286 | 84758 | 19:24643342 | 84760 | 19:24654902 | 84763 | 19:24710033 | 84766 | 19:24729440 |
| 84767 | 19:24730057 | 84768 | 19:24732020 | 84769 | 19:24736477 | 84770 | 19:24737388 | 84771 | 19:24739849 |
| 84772 | 19:24743924 | 84773 | 19:24747961 | 84774 | 19:24753662 | 84776 | 19:24761701 | 84777 | 19:24791465 |
| 84778 | 19:24832457 | 84779 | 19:24834421 | 84780 | 19:24843420 | 84782 | 19:24848682 | 84783 | 19:24859742 |
| 84784 | 19:24862477 | 84787 | 19:24938845 | 84788 | 19:24958116 | 84789 | 19:24958789 | 84793 | 19:25015319 |
| 84795 | 19:25042003 | 84797 | 19:25049455 | 84799 | 19:25055788 | 84802 | 19:25112769 | 84808 | 19:25148679 |
| 84809 | 19:25149070 | 84810 | 19:25167024 | 84815 | 19:25292038 | 84843 | 19:25636984 | 84847 | 19:25714307 |
| 84866 | 19:26042854 | 84867 | 19:26047036 | 84868 | 19:26047065 | 84869 | 19:26047497 | 84870 | 19:26047588 |
| 84871 | 19:26048186 | 84872 | 19:26048187 | 84873 | 19:26048200 | 84874 | 19:26048237 | 84875 | 19:26048249 |
| 84876 | 19:26050069 | 84877 | 19:26050076 | 84878 | 19:26050122 | 84894 | 19:26326260 | 84895 | 19:26328504 |
| 84896 | 19:26330382 | 84897 | 19:26362443 | 84903 | 19:26386801 | 84909 | 19:26431555 | 84911 | 19:26433035 |
| 84913 | 19:26435773 | 84914 | 19:26437827 | 84915 | 19:26438388 | 84919 | 19:26443714 | 84920 | 19:26452130 |
| 84921 | 19:26455955 | 84923 | 19:26459081 | 84924 | 19:26461611 | 84929 | 19:26467372 | 84933 | 19:26494500 |
| 84934 | 19:26498170 | 84937 | 19:26508308 | 84938 | 19:26509449 | 84940 | 19:26512520 | 84943 | 19:26571898 |
| 84967 | 19:26833517 | 84968 | 19:26834363 | 84971 | 19:26944800 | 84972 | 19:26944904 | 84991 | 19:27851735 |
| 84994 | 19:27919465 | 84995 | 19:27929802 | 84997 | 19:27974645 | 84999 | 19:27986943 | 85003 | 19:28063061 |
| 85022 | 19:28341985 | 85027 | 19:28441795 | 85033 | 19:28599438 | 85039 | 19:28789212 | 85055 | 19:29142555 |
| 85056 | 19:29146537 | 85057 | 19:29165645 | 85060 | 19:29185159 | 85062 | 19:29191189 | 85070 | 19:29360624 |
| 85071 | 19:29360635 | 85077 | 19:29516407 | 85082 | 19:29601656 | 85088 | 19:29667582 | 85089 | 19:29671837 |
| 85093 | 19:29784730 | 85094 | 19:29784751 | 85095 | 19:29784815 | 85106 | 19:30101489 | 85109 | 19:30199565 |
| 85125 | 19:30493276 | 85143 | 19:30892000 | 85145 | 19:30952848 | 85148 | 19:31087410 | 85150 | 19:31146132 |
| 85153 | 19:31161313 | 85154 | 19:31165405 | 85155 | 19:31165486 | 85160 | 19:31346087 | 85161 | 19:31369446 |
| 85193 | 19:32163965 | 85194 | 19:32172115 | 85201 | 19:32267525 | 85223 | 19:32751602 | 85224 | 19:32768693 |
| 85225 | 19:32800795 | 85226 | 19:32804878 | 85233 | 19:33007630 | 85234 | 19:33019809 | 85238 | 19:33125153 |
| 85243 | 19:33187115 | 85246 | 19:33215711 | 85249 | 19:33262257 | 85255 | 19:33294100 | 85267 | 19:33578331 |
| 85268 | 19:33594931 | 85269 | 19:33596340 | 85271 | 19:33623310 | 85279 | 19:33954621 | 85281 | 19:33998092 |
| 85282 | 19:34005543 | 85283 | 19:34005879 | 85286 | 19:34041049 | 85303 | 19:34565878 | 85306 | 19:34576319 |
| 85313 | 19:34769105 | 85314 | 19:34779947 | 85315 | 19:34782489 | 85316 | 19:34786152 | 85317 | 19:34795515 |
| 85318 | 19:34801199 | 85319 | 19:34814559 | 85320 | 19:34815409 | 85321 | 19:34821534 | 85322 | 19:34822575 |
| 85323 | 19:34823488 | 85324 | 19:34824385 | 85325 | 19:34825185 | 85326 | 19:34826088 | 85327 | 19:34826902 |
| 85328 | 19:34827822 | 85329 | 19:34832510 | 85330 | 19:34833809 | 85331 | 19:34835066 | 85332 | 19:34839003 |
| 85333 | 19:34839771 | 85334 | 19:34840668 | 85335 | 19:34842069 | 85336 | 19:34843150 | 85337 | 19:34850412 |
| 85338 | 19:34851901 | 85339 | 19:34860953 | 85340 | 19:34867998 | 85341 | 19:34873374 | 85342 | 19:34876608 |
| 85343 | 19:34877505 | 85344 | 19:34884414 | 85346 | 19:34893695 | 85348 | 19:34911757 | 85349 | 19:34917689 |
| 85350 | 19:34924937 | 85351 | 19:34940577 | 85352 | 19:34941647 | 85353 | 19:34942605 | 85360 | 19:35015390 |
| 85385 | 19:35630509 | 85391 | 19:35778297 | 85404 | 19:36050548 | 85405 | 19:36052759 | 85408 | 19:36139902 |
| 85410 | 19:36161222 | 85412 | 19:36183218 | 85413 | 19:36186920 | 85414 | 19:36204759 | 85415 | 19:36205924 |
| 85417 | 19:36230644 | 85418 | 19:36232203 | 85419 | 19:36233310 | 85420 | 19:36234144 | 85421 | 19:36235392 |
| 85422 | 19:36236022 | 85423 | 19:36236961 | 85424 | 19:36238112 | 85425 | 19:36239325 | 85426 | 19:36242008 |
| 85430 | 19:36285683 | 85432 | 19:36304660 | 85436 | 19:36349079 | 85443 | 19:36515692 | 85444 | 19:36516381 |
| 85445 | 19:36540830 | 85446 | 19:36572979 | 85447 | 19:36574100 | 85448 | 19:36574859 | 85449 | 19:36574986 |
| 85450 | 19:36576060 | 85451 | 19:36577180 | 85452 | 19:36577974 | 85453 | 19:36585052 | 85454 | 19:36585096 |
| 85455 | 19:36585153 | 85456 | 19:36586555 | 85457 | 19:36588245 | 85458 | 19:36598037 | 85460 | 19:36659503 |
| 85478 | 19:37057391 | 85480 | 19:37085911 | 85503 | 19:37768695 | 85512 | 19:38027080 | 85513 | 19:38112442 |
| 85514 | 19:38115441 | 85515 | 19:38135659 | 85519 | 19:38330215 | 85523 | 19:38378249 | 85535 | 19:38818642 |
| 85537 | 19:38825404 | 85543 | 19:39018067 | 85546 | 19:39173581 | 85548 | 19:39195126 | 85554 | 19:39319605 |
| 85555 | 19:39319939 | 85556 | 19:39324677 | 85557 | 19:39373787 | 85558 | 19:39403214 | 85560 | 19:39423726 |
| 85564 | 19:39558542 | 85568 | 19:39592912 | 85572 | 19:39664178 | 85574 | 19:39702056 | 85575 | 19:39761996 |
| 85585 | 19:40136281 | 85589 | 19:40231461 | 85591 | 19:40243945 | 85592 | 19:40244440 | 85593 | 19:40244746 |
| 85594 | 19:40250106 | 85603 | 19:40340447 | 85608 | 19:40504497 | 85611 | 19:40571544 | 85617 | 19:40687629 |
| 85634 | 19:40969791 | 85636 | 19:40998269 | 85637 | 19:41009813 | 85639 | 19:41075395 | 85641 | 19:41125615 |
| 85642 | 19:41125654 | 85643 | 19:41126901 | 85644 | 19:41134261 | 85645 | 19:41134335 | 85646 | 19:41167136 |
| 85666 | 19:41672120 | 85671 | 19:41759325 | 85681 | 19:41844566 | 85683 | 19:41866585 | 85685 | 19:41908524 |
| 85687 | 19:41944555 | 85689 | 19:41954155 | 85694 | 19:42058566 | 85695 | 19:42063521 | 85698 | 19:42131015 |
| 85701 | 19:42144465 | 85704 | 19:42227260 | 85710 | 19:42333202 | 85712 | 19:42345437 | 85719 | 19:42426465 |
| 85728 | 19:42644624 | 85731 | 19:42719942 | 85732 | 19:42726195 | 85736 | 19:42848843 | 85738 | 19:42887598 |
| 85741 | 19:43035501 | 85742 | 19:43043947 | 85743 | 19:43049014 | 85750 | 19:43333514 | 85759 | 19:43422824 |
| 85768 | 19:43443947 | 85775 | 19:43599080 | 85777 | 19:43713425 | 85783 | 19:43816258 | 85784 | 19:43830215 |
| 85787 | 19:43926648 | 85790 | 19:43991252 | 85793 | 19:44072509 | 85795 | 19:44116524 | 85797 | 19:44122310 |
| 85799 | 19:44173520 | 85800 | 19:44177664 | 85801 | 19:44178974 | 85806 | 19:44309222 | 85808 | 19:44359879 |
| 85809 | 19:44385146 | 85813 | 19:44477082 | 85818 | 19:44577139 | 85821 | 19:44615861 | 85822 | 19:44651270 |
| 85823 | 19:44673344 | 85824 | 19:44673867 | 85826 | 19:44715813 | 85838 | 19:45013727 | 85841 | 19:45073651 |
| 85861 | 19:45630233 | 85892 | 19:45816366 | 85893 | 19:45817041 | 85896 | 19:45844326 | 85898 | 19:45920605 |
| 85899 | 19:45934999 | 85900 | 19:45949710 | 85909 | 19:46106226 | 85918 | 19:46396057 | 85928 | 19:46762829 |
| 85933 | 19:46867389 | 85944 | 19:47138439 | 85945 | 19:47138441 | 85946 | 19:47138654 | 85947 | 19:47138655 |
| 85948 | 19:47138674 | 85949 | 19:47138684 | 85950 | 19:47139281 | 85958 | 19:47384175 | 85959 | 19:47402508 |
| 85960 | 19:47405017 | 85974 | 19:47676904 | 85975 | 19:47735369 | 85979 | 19:47763256 | 85984 | 19:47930272 |
| 85986 | 19:47955876 | 85987 | 19:47966555 | 85990 | 19:48025233 | 85993 | 19:48071339 | 85994 | 19:48075239 |
| 85996 | 19:48084284 | 86000 | 19:48118256 | 86001 | 19:48142220 | 86005 | 19:48258274 | 86006 | 19:48266963 |
| 86012 | 19:48389397 | 86016 | 19:48418566 | 86022 | 19:48563047 | 86028 | 19:48709127 | 86032 | 19:48880795 |
| 86042 | 19:49040357 | 86051 | 19:49233318 | 86055 | 19:49277856 | 86063 | 19:49478759 | 86064 | 19:49483816 |
| 86065 | 19:49494727 | 86068 | 19:49575523 | 86076 | 19:49772893 | 86078 | 19:49810028 | 86085 | 19:49891419 |
| 86092 | 19:50063999 | 86094 | 19:50095971 | 86095 | 19:50107587 | 86096 | 19:50123299 | 86099 | 19:50167379 |
| 86100 | 19:50172634 | 86101 | 19:50173024 | 86102 | 19:50173036 | 86103 | 19:50173360 | 86104 | 19:50173420 |
| 86116 | 19:50607587 | 86118 | 19:50662334 | 86122 | 19:50682193 | 86139 | 19:50783155 | 86140 | 19:50784393 |
| 86142 | 19:50788319 | 86144 | 19:50790149 | 86145 | 19:50790973 | 86146 | 19:50791045 | 86147 | 19:50817214 |
| 86148 | 19:50817251 | 86150 | 19:50838297 | 86151 | 19:50838402 | 86152 | 19:50855700 | 86153 | 19:50856195 |
| 86154 | 19:50883463 | 86155 | 19:50890139 | 86156 | 19:50892965 | 86157 | 19:50893275 | 86158 | 19:50893330 |
| 86159 | 19:50893412 | 86160 | 19:50894507 | 86164 | 19:51146306 | 86165 | 19:51148044 | 86166 | 19:51150289 |
| 86167 | 19:51151634 | 86168 | 19:51152336 | 86169 | 19:51152846 | 86170 | 19:51153433 | 86171 | 19:51155662 |
| 86172 | 19:51156301 | 86173 | 19:51158110 | 86174 | 19:51172862 | 86175 | 19:51173451 | 86176 | 19:51179705 |
| 86177 | 19:51181597 | 86178 | 19:51184543 | 86179 | 19:51185390 | 86180 | 19:51186411 | 86181 | 19:51186781 |
| 86182 | 19:51186933 | 86183 | 19:51187678 | 86184 | 19:51192006 | 86185 | 19:51194044 | 86186 | 19:51195370 |
| 86187 | 19:51196420 | 86188 | 19:51197290 | 86189 | 19:51197844 | 86190 | 19:51198485 | 86191 | 19:51200087 |
| 86192 | 19:51202775 | 86193 | 19:51203906 | 86194 | 19:51204912 | 86195 | 19:51206147 | 86196 | 19:51206982 |
| 86197 | 19:51208278 | 86198 | 19:51209192 | 86199 | 19:51215165 | 86213 | 19:51584353 | 86214 | 19:51614996 |
| 86215 | 19:51624830 | 86219 | 19:51657080 | 86220 | 19:51674934 | 86221 | 19:51674957 | 86222 | 19:51674972 |
| 86223 | 19:51676635 | 86225 | 19:51688593 | 86226 | 19:51689866 | 86238 | 19:52025573 | 86243 | 19:52232662 |
| 86246 | 19:52242008 | 86261 | 19:52716129 | 86275 | 19:52957714 | 86277 | 19:53006255 | 86279 | 19:53041756 |
| 86281 | 19:53068039 | 86285 | 19:53125990 | 86286 | 19:53132946 | 86301 | 19:53433970 | 86302 | 19:53439232 |
| 86304 | 19:53484305 | 86308 | 19:53566656 | 86309 | 19:53572790 | 86310 | 19:53588198 | 86314 | 19:53698415 |
| 86318 | 19:53748765 | 86320 | 19:53774472 | 86321 | 19:53778116 | 86323 | 19:53811161 | 86333 | 19:53948075 |
| 86337 | 19:54028347 | 86338 | 19:54043604 | 86341 | 19:54053040 | 86352 | 19:54289712 | 86353 | 19:54295559 |
| 86355 | 19:54301176 | 86358 | 19:54390593 | 86371 | 19:54622173 | 86372 | 19:54637181 | 86379 | 19:54710734 |
| 86383 | 19:54755988 | 86388 | 19:54858324 | 86393 | 19:54978600 | 86398 | 19:55092867 | 86403 | 19:55229692 |
| 86406 | 19:55265827 | 86409 | 19:55330553 | 86413 | 19:55427978 | 86414 | 19:55428029 | 86422 | 19:55580085 |
| 86427 | 19:55698969 | 86432 | 19:55792180 | 86433 | 19:55793483 | 86434 | 19:55793574 | 86436 | 19:55827900 |
| 86437 | 19:55830150 | 86440 | 19:55848361 | 86441 | 19:55848663 | 86444 | 19:55866969 | 86449 | 19:55906352 |
| 86452 | 19:55911397 | 86454 | 19:55915911 | 86455 | 19:55917441 | 86456 | 19:55920727 | 86458 | 19:55922694 |
| 86462 | 19:55926659 | 86464 | 19:55940240 | 86471 | 19:55985092 | 86472 | 19:55985151 | 86473 | 19:55985483 |
| 86474 | 19:55986867 | 86475 | 19:55986948 | 86477 | 19:55987569 | 86479 | 19:55988850 | 86480 | 19:55988905 |
| 86481 | 19:55988950 | 86482 | 19:55989161 | 86483 | 19:55989317 | 86484 | 19:55989324 | 86486 | 19:55993442 |
| 86489 | 19:56009506 | 86495 | 19:56024722 | 86500 | 19:56044293 | 86503 | 19:56059597 | 86512 | 19:56100545 |
| 86516 | 19:56104113 | 86535 | 19:56318677 | 86536 | 19:56320176 | 86537 | 19:56346313 | 86538 | 19:56346447 |
| 86539 | 19:56348743 | 86540 | 19:56388703 | 86544 | 19:56447130 | 86549 | 19:56504478 | 86550 | 19:56506885 |
| 86551 | 19:56506918 | 86553 | 19:56521721 | 86554 | 19:56527708 | 86556 | 19:56536304 | 86558 | 19:56542031 |
| 86559 | 19:56544120 | 86560 | 19:56545458 | 86561 | 19:56546864 | 86562 | 19:56553455 | 86571 | 19:56792481 |
| 86573 | 19:56794455 | 86576 | 19:56891022 | 86579 | 19:56962292 | 86581 | 19:57011124 | 86582 | 19:57012570 |
| 86591 | 19:57196446 | 86592 | 19:57214845 | 86593 | 19:57259057 | 86600 | 19:57418240 | 86608 | 19:57566385 |
| 86631 | 19:58091496 | 86632 | 19:58091529 | 86634 | 19:58127830 | 86638 | 19:58214009 | 86639 | 19:58218020 |
| 86645 | 19:58304430 | 86650 | 19:58429115 | 86659 | 19:58594514 | 86665 | 19:58750938 | 86673 | 19:58918004 |
| 86688 | 19:59274219 | 86689 | 19:59281932 | 86711 | 19:59592874 | 86718 | 19:59660094 | 86724 | 19:59743571 |
| 86727 | 19:59784192 | 86731 | 19:59871909 | 86732 | 19:59872022 | 86742 | 19:59984597 | 86756 | 19:60173328 |
| 86768 | 19:60319323 | 86776 | 19:60416386 | 86781 | 19:60493532 | 86782 | 19:60495581 | 86793 | 19:60643681 |
| 86795 | 19:60660851 | 86800 | 19:60743931 | 86802 | 19:60816246 | 86811 | 19:60977087 | 86814 | 19:61065143 |
| 86818 | 19:61145338 | 86819 | 19:61146374 | 86822 | 19:61175458 | 86823 | 19:61175494 | 86824 | 19:61175495 |
| 86825 | 19:61175593 | 86826 | 19:61175611 | 86834 | 19:61242337 | 86845 | 19:61459019 | 86846 | 19:61474017 |
| 86848 | 19:61529467 | 86849 | 19:61531654 | 86850 | 19:61531677 | 86851 | 19:61532390 | 86852 | 19:61533739 |
| 86854 | 19:61538770 | 86862 | 19:61639102 | 86865 | 19:61681705 | 86867 | 19:61701942 | 86868 | 19:61709842 |
| 86874 | 19:61794791 | 86884 | 19:61956159 | 86885 | 19:61968113 | 86892 | 19:62135323 | 86904 | 19:62322264 |
| 86915 | 19:62611634 | 86916 | 19:62620305 | 86919 | 19:62738018 | 86927 | 19:62774324 | 86928 | 19:62785952 |
| 86942 | 19:63013941 | 86944 | 19:63088763 | 86946 | 19:63091044 | 86959 | 19:63394562 | 86960 | 19:63394839 |
| 86965 | 20:283185 | 86975 | 20:495073 | 86979 | 20:523765 | 86989 | 20:666472 | 87009 | 20:1104356 |
| 87012 | 20:1164612 | 87016 | 20:1365586 | 87019 | 20:1476960 | 87020 | 20:1477326 | 87025 | 20:1585296 |
| 87031 | 20:1627743 | 87032 | 20:1627820 | 87033 | 20:1628846 | 87038 | 20:1708322 | 87040 | 20:1708393 |
| 87051 | 20:1881180 | 87054 | 20:1890880 | 87058 | 20:1964788 | 87059 | 20:1981954 | 87060 | 20:1985087 |
| 87062 | 20:2008634 | 87066 | 20:2111352 | 87071 | 20:2174269 | 87082 | 20:2329012 | 87084 | 20:2363600 |
| 87090 | 20:2486996 | 87093 | 20:2549449 | 87094 | 20:2549482 | 87123 | 20:3122350 | 87125 | 20:3132189 |
| 87126 | 20:3144963 | 87127 | 20:3147090 | 87128 | 20:3149103 | 87133 | 20:3256361 | 87141 | 20:3434776 |
| 87143 | 20:3442837 | 87144 | 20:3490573 | 87153 | 20:3643964 | 87163 | 20:3970013 | 87184 | 20:4419380 |
| 87186 | 20:4422606 | 87187 | 20:4422634 | 87189 | 20:4471095 | 87201 | 20:4600219 | 87353 | 20:4974566 |
| 87356 | 20:4978213 | 87396 | 20:5081279 | 87397 | 20:5081368 | 87398 | 20:5081429 | 87411 | 20:5256314 |
| 87424 | 20:5427984 | 87425 | 20:5449185 | 87427 | 20:5456791 | 87445 | 20:5952561 | 87471 | 20:6124117 |
| 87472 | 20:6127051 | 87473 | 20:6128007 | 87474 | 20:6129222 | 87475 | 20:6135444 | 87478 | 20:6222819 |
| 87491 | 20:6538447 | 87493 | 20:6552399 | 87502 | 20:6787304 | 87503 | 20:6803666 | 87504 | 20:6804954 |
| 87505 | 20:6809070 | 87507 | 20:6819134 | 87508 | 20:6824958 | 87514 | 20:7026132 | 87515 | 20:7028111 |
| 87527 | 20:7184948 | 87529 | 20:7203346 | 87530 | 20:7228830 | 87536 | 20:7397715 | 87538 | 20:7452989 |
| 87556 | 20:7937085 | 87558 | 20:7966207 | 87565 | 20:8099607 | 87580 | 20:8406030 | 87588 | 20:8596907 |
| 87591 | 20:8676605 | 87597 | 20:8915848 | 87599 | 20:8976316 | 87600 | 20:8994477 | 87603 | 20:9008027 |
| 87604 | 20:9008070 | 87610 | 20:9122965 | 87611 | 20:9185096 | 87616 | 20:9277064 | 87617 | 20:9284840 |
| 87618 | 20:9286211 | 87619 | 20:9286666 | 87620 | 20:9297979 | 87622 | 20:9309443 | 87627 | 20:9387110 |
| 87635 | 20:9562025 | 87642 | 20:9678407 | 87643 | 20:9699294 | 87653 | 20:9987753 | 87657 | 20:10105910 |
| 87670 | 20:10568285 | 87677 | 20:10719241 | 87691 | 20:11104981 | 87695 | 20:11173739 | 87708 | 20:11384109 |
| 87709 | 20:11423657 | 87712 | 20:11441654 | 87719 | 20:11603501 | 87723 | 20:11652136 | 87726 | 20:11746445 |
| 87727 | 20:11748411 | 87729 | 20:11801270 | 87732 | 20:11873589 | 87736 | 20:12001911 | 87745 | 20:12195219 |
| 87751 | 20:12336592 | 87756 | 20:12431016 | 87759 | 20:12454756 | 87760 | 20:12470759 | 87770 | 20:12619125 |
| 87776 | 20:12725086 | 87777 | 20:12732314 | 87787 | 20:12872002 | 87788 | 20:12884968 | 87792 | 20:13038217 |
| 87800 | 20:13212537 | 87804 | 20:13314745 | 87806 | 20:13360616 | 87808 | 20:13384250 | 87809 | 20:13411239 |
| 87820 | 20:13846644 | 87821 | 20:13853397 | 87827 | 20:13967913 | 87881 | 20:16013654 | 87890 | 20:16311925 |
| 87891 | 20:16312024 | 87892 | 20:16312059 | 87893 | 20:16312062 | 87926 | 20:17161518 | 87940 | 20:17529786 |
| 87946 | 20:17718360 | 87949 | 20:17740634 | 87950 | 20:17772296 | 87959 | 20:18106764 | 87966 | 20:18199987 |
| 87974 | 20:18560092 | 87979 | 20:18738258 | 87981 | 20:18767430 | 87988 | 20:18999163 | 87998 | 20:19219710 |
| 88010 | 20:19525255 | 88013 | 20:19638381 | 88024 | 20:19910542 | 88026 | 20:19934333 | 88029 | 20:19951507 |
| 88030 | 20:19985004 | 88035 | 20:20229781 | 88049 | 20:20735095 | 88053 | 20:20833870 | 88054 | 20:20847713 |
| 88055 | 20:20852713 | 88057 | 20:20900999 | 88058 | 20:20916403 | 88060 | 20:20932871 | 88062 | 20:20985150 |
| 88069 | 20:21165648 | 88070 | 20:21199073 | 88076 | 20:21442552 | 88119 | 20:22069390 | 88123 | 20:22130295 |
| 88158 | 20:22204908 | 88186 | 20:22465821 | 88204 | 20:22827690 | 88213 | 20:23009076 | 88214 | 20:23038319 |
| 88215 | 20:23039137 | 88224 | 20:23272824 | 88234 | 20:23534898 | 88236 | 20:23571173 | 88237 | 20:23574812 |
| 88238 | 20:23575304 | 88242 | 20:23686099 | 88243 | 20:23692874 | 88248 | 20:23836890 | 88249 | 20:23837832 |
| 88250 | 20:23838131 | 88251 | 20:23838181 | 88252 | 20:23841545 | 88253 | 20:23861537 | 88254 | 20:23861790 |
| 88255 | 20:23862521 | 88256 | 20:23863171 | 88257 | 20:23864163 | 88258 | 20:23864320 | 88260 | 20:23868037 |
| 88261 | 20:23872171 | 88262 | 20:23872177 | 88263 | 20:23873363 | 88265 | 20:23931936 | 88266 | 20:23934587 |
| 88267 | 20:23936814 | 88268 | 20:23937494 | 88269 | 20:23940208 | 88270 | 20:23940805 | 88271 | 20:23948706 |
| 88272 | 20:23950019 | 88273 | 20:23956567 | 88274 | 20:23967747 | 88278 | 20:24005106 | 88283 | 20:24183792 |
| 88289 | 20:24300670 | 88291 | 20:24342520 | 88301 | 20:24551449 | 88303 | 20:24600379 | 88304 | 20:24644625 |
| 88314 | 20:24840568 | 88316 | 20:24888410 | 88336 | 20:25687633 | 88341 | 20:25956846 | 88353 | 20:26326568 |
| 88377 | 20:26904319 | 88394 | 20:27389017 | 88396 | 20:27389119 | 88418 | 20:27942740 | 88423 | 20:28062228 |
| 88424 | 20:28062230 | 88425 | 20:28067347 | 88435 | 20:28291529 | 88440 | 20:28407293 | 88449 | 20:28594465 |
| 88452 | 20:28714693 | 88453 | 20:28719941 | 88454 | 20:28719958 | 88455 | 20:28720052 | 88456 | 20:28726509 |
| 88457 | 20:28726541 | 88458 | 20:28726553 | 88459 | 20:28726570 | 88460 | 20:28726626 | 88468 | 20:28835792 |
| 88474 | 20:28977237 | 88475 | 20:28978238 | 88488 | 20:29362772 | 88489 | 20:29376013 | 88491 | 20:29447835 |
| 88508 | 20:29990492 | 88509 | 20:29990621 | 88514 | 20:30118413 | 88518 | 20:30200252 | 88520 | 20:30212011 |
| 88532 | 20:30669895 | 88536 | 20:30689871 | 88539 | 20:30825518 | 88546 | 20:31205981 | 88547 | 20:31211377 |
| 88548 | 20:31243755 | 88549 | 20:31245339 | 88558 | 20:31456913 | 88567 | 20:31684137 | 88570 | 20:31752397 |
| 88576 | 20:31881419 | 88579 | 20:31903713 | 88582 | 20:31972125 | 88585 | 20:32052139 | 88588 | 20:32101132 |
| 88597 | 20:32409103 | 88633 | 20:33398481 | 88637 | 20:33502768 | 88640 | 20:33565104 | 88641 | 20:33569466 |
| 88648 | 20:33719114 | 88653 | 20:33836807 | 88657 | 20:34001523 | 88663 | 20:34145466 | 88666 | 20:34184342 |
| 88669 | 20:34272037 | 88692 | 20:34738766 | 88694 | 20:34781170 | 88697 | 20:34913079 | 88714 | 20:35421048 |
| 88721 | 20:35551560 | 88722 | 20:35585029 | 88723 | 20:35610755 | 88726 | 20:35701545 | 88728 | 20:35715693 |
| 88736 | 20:35846193 | 88737 | 20:35882733 | 88741 | 20:35994055 | 88742 | 20:35994063 | 88743 | 20:35996784 |
| 88762 | 20:36319426 | 88767 | 20:36496636 | 88776 | 20:36648116 | 88781 | 20:36787045 | 88782 | 20:36834159 |
| 88783 | 20:36858954 | 88785 | 20:36892081 | 88788 | 20:36968962 | 88789 | 20:36989703 | 88791 | 20:37042637 |
| 88794 | 20:37085325 | 88795 | 20:37092573 | 88797 | 20:37131660 | 88798 | 20:37131690 | 88799 | 20:37136413 |
| 88804 | 20:37341795 | 88810 | 20:37443371 | 88811 | 20:37443444 | 88812 | 20:37443481 | 88813 | 20:37473672 |
| 88815 | 20:37479797 | 88816 | 20:37481956 | 88827 | 20:37694264 | 88828 | 20:37694333 | 88831 | 20:37811388 |
| 88832 | 20:37811427 | 88833 | 20:37811480 | 88834 | 20:37811543 | 88838 | 20:37887781 | 88845 | 20:38177941 |
| 88846 | 20:38189067 | 88867 | 20:38758576 | 88881 | 20:39088930 | 88882 | 20:39109157 | 88887 | 20:39238537 |
| 88888 | 20:39245057 | 88890 | 20:39287657 | 88893 | 20:39383171 | 88894 | 20:39383264 | 88898 | 20:39445232 |
| 88904 | 20:39475943 | 88909 | 20:39539960 | 88911 | 20:39555296 | 88912 | 20:39557649 | 88924 | 20:39810901 |
| 88925 | 20:39863071 | 88928 | 20:39910970 | 88930 | 20:39965197 | 88937 | 20:40059831 | 88941 | 20:40215915 |
| 88955 | 20:40683858 | 88956 | 20:40684807 | 88957 | 20:40695016 | 88961 | 20:40772462 | 88966 | 20:40834506 |
| 88968 | 20:40945725 | 88975 | 20:41050907 | 88976 | 20:41050966 | 88978 | 20:41100712 | 88984 | 20:41160179 |
| 88986 | 20:41192863 | 88990 | 20:41314439 | 89010 | 20:41798915 | 89012 | 20:41819893 | 89013 | 20:41823837 |
| 89016 | 20:41845476 | 89023 | 20:41919178 | 89028 | 20:42044095 | 89029 | 20:42045334 | 89032 | 20:42085412 |
| 89035 | 20:42133834 | 89039 | 20:42246244 | 89042 | 20:42293890 | 89043 | 20:42331127 | 89047 | 20:42565028 |
| 89061 | 20:42942479 | 89066 | 20:43159533 | 89079 | 20:43561846 | 89080 | 20:43561856 | 89101 | 20:44025947 |
| 89102 | 20:44026070 | 89107 | 20:44281699 | 89108 | 20:44301844 | 89121 | 20:44556774 | 89122 | 20:44574229 |
| 89123 | 20:44591612 | 89133 | 20:44966449 | 89134 | 20:44969446 | 89145 | 20:45365363 | 89186 | 20:46291117 |
| 89194 | 20:46578492 | 89208 | 20:47160581 | 89209 | 20:47160583 | 89211 | 20:47163202 | 89212 | 20:47163219 |
| 89243 | 20:48405434 | 89244 | 20:48405481 | 89245 | 20:48405501 | 89246 | 20:48405505 | 89247 | 20:48405507 |
| 89248 | 20:48405524 | 89249 | 20:48405575 | 89251 | 20:48474578 | 89262 | 20:48874528 | 89264 | 20:48881242 |
| 89265 | 20:48881303 | 89266 | 20:48881343 | 89267 | 20:48901928 | 89274 | 20:49054367 | 89297 | 20:49861661 |
| 89298 | 20:49913380 | 89299 | 20:49915377 | 89300 | 20:49918452 | 89323 | 20:50694458 | 89328 | 20:50806661 |
| 89338 | 20:51034291 | 89363 | 20:51929370 | 89385 | 20:52736752 | 89394 | 20:52981985 | 89401 | 20:53204896 |
| 89405 | 20:53347849 | 89415 | 20:53521298 | 89416 | 20:53526124 | 89420 | 20:53570726 | 89428 | 20:53840361 |
| 89442 | 20:54395462 | 89465 | 20:55054760 | 89466 | 20:55055902 | 89478 | 20:55217698 | 89485 | 20:55403499 |
| 89500 | 20:55900412 | 89503 | 20:55938086 | 89522 | 20:56361024 | 89526 | 20:56443806 | 89530 | 20:56569222 |
| 89532 | 20:56609894 | 89533 | 20:56609977 | 89536 | 20:56692099 | 89541 | 20:56762906 | 89570 | 20:57486934 |
| 89571 | 20:57487439 | 89576 | 20:57572314 | 89584 | 20:57778974 | 89595 | 20:57998700 | 89596 | 20:57998708 |
| 89597 | 20:57998777 | 89598 | 20:57998802 | 89599 | 20:57998807 | 89600 | 20:57998808 | 89601 | 20:57998848 |
| 89602 | 20:57998909 | 89604 | 20:58038602 | 89605 | 20:58038638 | 89606 | 20:58038676 | 89610 | 20:58171888 |
| 89622 | 20:58370776 | 89623 | 20:58371818 | 89628 | 20:58425428 | 89630 | 20:58432597 | 89638 | 20:58662715 |
| 89652 | 20:58839976 | 89653 | 20:58841417 | 89661 | 20:58985542 | 89665 | 20:59056598 | 89668 | 20:59062072 |
| 89675 | 20:59262668 | 89696 | 20:59893295 | 89698 | 20:59918108 | 89699 | 20:59918158 | 89700 | 20:59918229 |
| 89702 | 20:59984000 | 89750 | 20:61610794 | 89751 | 20:61610854 | 89755 | 20:61693804 | 89767 | 20:61962563 |
| 89773 | 20:62086784 | 89786 | 20:62394644 | 89790 | 20:62513410 | 89792 | 20:62528844 | 89794 | 20:62575332 |
| 89799 | 20:62653294 | 89800 | 20:62670652 | 89808 | 20:62848870 | 89810 | 20:62914219 | 89811 | 20:62937049 |
| 89812 | 20:62942539 | 89813 | 20:62963027 | 89815 | 20:62971781 | 89828 | 20:63314880 | 89830 | 20:63382028 |
| 89831 | 20:63384182 | 89841 | 20:63652842 | 89843 | 20:63711747 | 89848 | 20:63813002 | 89857 | 20:63923149 |
| 89858 | 20:63934159 | 89859 | 20:63939074 | 89860 | 20:63939894 | 89861 | 20:63949508 | 89862 | 20:63950751 |
| 89863 | 20:63952037 | 89864 | 20:63952862 | 89865 | 20:63954238 | 89866 | 20:63981759 | 89871 | 20:64108375 |
| 89891 | 20:64483792 | 89912 | 20:65101244 | 89919 | 20:65253620 | 89927 | 20:65566194 | 89928 | 20:65578450 |
| 89929 | 20:65592431 | 89932 | 20:65619064 | 89934 | 20:65691397 | 89936 | 20:65761375 | 89937 | 20:65773012 |
| 89938 | 20:65786774 | 89945 | 20:65973669 | 89950 | 20:66089403 | 89955 | 20:66319547 | 89959 | 20:66370625 |
| 89960 | 20:66372293 | 89962 | 20:66457113 | 89963 | 20:66464862 | 89964 | 20:66468697 | 89967 | 20:66551247 |
| 89968 | 20:66556459 | 89969 | 20:66563281 | 89970 | 20:66563428 | 89971 | 20:66567783 | 89972 | 20:66568233 |
| 89973 | 20:66626549 | 89976 | 20:66691115 | 89978 | 20:66696604 | 89979 | 20:66711575 | 89980 | 20:66711602 |
| 90001 | 20:67153888 | 90012 | 20:67465264 | 90016 | 20:67521067 | 90026 | 20:67735557 | 90027 | 20:67739397 |
| 90028 | 20:67743005 | 90031 | 20:67808126 | 90032 | 20:67819595 | 90035 | 20:67872456 | 90039 | 20:67995355 |
| 90041 | 20:68014834 | 90054 | 20:68168592 | 90062 | 20:68323418 | 90063 | 20:68352541 | 90071 | 20:68463408 |
| 90072 | 20:68503575 | 90079 | 20:68640146 | 90087 | 20:68746121 | 90095 | 20:68908716 | 90102 | 20:68990075 |
| 90129 | 20:69518490 | 90157 | 20:70210220 | 90182 | 20:70941731 | 90183 | 20:70948562 | 90184 | 20:70949203 |
| 90185 | 20:70954723 | 90187 | 20:70972905 | 90188 | 20:70973702 | 90189 | 20:70975538 | 90193 | 20:71082114 |
| 90194 | 20:71097489 | 90195 | 20:71099307 | 90196 | 20:71104986 | 90197 | 20:71108601 | 90198 | 20:71110857 |
| 90199 | 20:71113969 | 90200 | 20:71118893 | 90201 | 20:71119829 | 90202 | 20:71121420 | 90203 | 20:71122291 |
| 90204 | 20:71124183 | 90205 | 20:71124516 | 90206 | 20:71127650 | 90207 | 20:71131787 | 90208 | 20:71139430 |
| 90209 | 20:71141395 | 90210 | 20:71147142 | 90211 | 20:71147843 | 90212 | 20:71150904 | 90213 | 20:71153153 |
| 90214 | 20:71154456 | 90215 | 20:71162376 | 90216 | 20:71172637 | 90217 | 20:71173872 | 90218 | 20:71177834 |
| 90219 | 20:71180101 | 90220 | 20:71181518 | 90221 | 20:71183377 | 90222 | 20:71188295 | 90223 | 20:71195927 |
| 90224 | 20:71201866 | 90225 | 20:71203976 | 90226 | 20:71206269 | 90227 | 20:71207699 | 90228 | 20:71212077 |
| 90229 | 20:71232016 | 90230 | 20:71240516 | 90231 | 20:71290453 | 90232 | 20:71293773 | 90233 | 20:71295284 |
| 90234 | 20:71300630 | 90235 | 20:71301433 | 90236 | 20:71313672 | 90237 | 20:71319974 | 90238 | 20:71320628 |
| 90239 | 20:71349322 | 90240 | 20:71354558 | 90241 | 20:71355354 | 90242 | 20:71359918 | 90243 | 20:71360768 |
| 90244 | 20:71378946 | 90245 | 20:71385619 | 90246 | 20:71386554 | 90247 | 20:71387739 | 90248 | 20:71388363 |
| 90249 | 20:71393864 | 90250 | 20:71395443 | 90251 | 20:71396187 | 90252 | 20:71401124 | 90254 | 20:71402777 |
| 90255 | 20:71403559 | 90256 | 20:71407893 | 90257 | 20:71417623 | 90258 | 20:71431631 | 90259 | 20:71442260 |
| 90260 | 20:71444098 | 90261 | 20:71447274 | 90262 | 20:71448297 | 90263 | 20:71448849 | 90264 | 20:71450177 |
| 90265 | 20:71450861 | 90266 | 20:71459002 | 90267 | 20:71472317 | 90268 | 20:71490172 | 90269 | 20:71491300 |
| 90272 | 20:71606988 | 90280 | 20:71927868 | 90305 | 21:1606872 | 90320 | 21:2377105 | 90330 | 21:2684106 |
| 90336 | 21:2874100 | 90344 | 21:3001448 | 90366 | 21:3866539 | 90375 | 21:4053839 | 90377 | 21:4055731 |
| 90378 | 21:4060826 | 90402 | 21:4420712 | 90413 | 21:4948777 | 90420 | 21:5153009 | 90425 | 21:5327112 |
| 90428 | 21:5382657 | 90436 | 21:5552825 | 90437 | 21:5575472 | 90441 | 21:5776117 | 90449 | 21:5865604 |
| 90453 | 21:5946488 | 90454 | 21:5946554 | 90455 | 21:5961487 | 90460 | 21:6096270 | 90469 | 21:6466492 |
| 90471 | 21:6515336 | 90477 | 21:6718976 | 90479 | 21:6821186 | 90480 | 21:6838984 | 90481 | 21:6864243 |
| 90488 | 21:7110184 | 90489 | 21:7126782 | 90506 | 21:7582228 | 90508 | 21:7589812 | 90509 | 21:7590462 |
| 90520 | 21:7863440 | 90521 | 21:7868053 | 90524 | 21:7907745 | 90530 | 21:8033587 | 90534 | 21:8074100 |
| 90535 | 21:8074643 | 90552 | 21:8176568 | 90554 | 21:8190635 | 90555 | 21:8226423 | 90562 | 21:8310287 |
| 90604 | 21:9465633 | 90628 | 21:9948207 | 90632 | 21:10010929 | 90642 | 21:10296960 | 90647 | 21:10509482 |
| 90653 | 21:10609946 | 90654 | 21:10624234 | 90671 | 21:10957495 | 90679 | 21:11069313 | 90693 | 21:11440159 |
| 90694 | 21:11445014 | 90698 | 21:11556518 | 90703 | 21:11654700 | 90743 | 21:12681133 | 90749 | 21:12760689 |
| 90754 | 21:12855624 | 90776 | 21:13353466 | 90780 | 21:13514487 | 90794 | 21:13810003 | 90798 | 21:13905626 |
| 90805 | 21:14029521 | 90811 | 21:14202552 | 90823 | 21:14517580 | 90824 | 21:14560006 | 90834 | 21:14713608 |
| 90840 | 21:14945241 | 90848 | 21:15132400 | 90849 | 21:15138571 | 90851 | 21:15156831 | 90852 | 21:15156885 |
| 90853 | 21:15156911 | 90856 | 21:15219073 | 90859 | 21:15326382 | 90888 | 21:15932209 | 90894 | 21:16294354 |
| 90896 | 21:16311821 | 90897 | 21:16324443 | 90901 | 21:16376470 | 90905 | 21:16407620 | 90906 | 21:16426000 |
| 90908 | 21:16455704 | 90912 | 21:16482563 | 90914 | 21:16507141 | 90919 | 21:16581532 | 90920 | 21:16583122 |
| 90922 | 21:16605546 | 90930 | 21:16771714 | 90932 | 21:16775550 | 90933 | 21:16780485 | 90940 | 21:16994759 |
| 90945 | 21:17105820 | 90947 | 21:17157614 | 90967 | 21:17696116 | 91010 | 21:18775346 | 91011 | 21:18792766 |
| 91012 | 21:18796836 | 91013 | 21:18806197 | 91014 | 21:18807592 | 91015 | 21:18807654 | 91018 | 21:18889130 |
| 91019 | 21:18889216 | 91020 | 21:18889225 | 91021 | 21:18897548 | 91022 | 21:18897617 | 91023 | 21:18897699 |
| 91024 | 21:18906129 | 91025 | 21:18906131 | 91035 | 21:19086207 | 91044 | 21:19274749 | 91045 | 21:19274809 |
| 91049 | 21:19328532 | 91050 | 21:19341878 | 91058 | 21:19448733 | 91059 | 21:19484240 | 91061 | 21:19509806 |
| 91065 | 21:19547342 | 91066 | 21:19566627 | 91067 | 21:19571760 | 91068 | 21:19574086 | 91069 | 21:19574861 |
| 91071 | 21:19581384 | 91072 | 21:19587859 | 91074 | 21:19591585 | 91077 | 21:19636076 | 91078 | 21:19650309 |
| 91079 | 21:19744871 | 91080 | 21:19745043 | 91081 | 21:19765456 | 91082 | 21:19765736 | 91083 | 21:19765864 |
| 91084 | 21:19765895 | 91085 | 21:19765940 | 91088 | 21:19791905 | 91089 | 21:19796807 | 91090 | 21:19800176 |
| 91092 | 21:19819891 | 91093 | 21:19837076 | 91120 | 21:20576639 | 91123 | 21:20597014 | 91124 | 21:20611156 |
| 91129 | 21:20643657 | 91132 | 21:20661675 | 91137 | 21:20707916 | 91139 | 21:20756163 | 91140 | 21:20767918 |
| 91148 | 21:20994233 | 91149 | 21:20997368 | 91150 | 21:21002238 | 91153 | 21:21051410 | 91154 | 21:21051969 |
| 91155 | 21:21079825 | 91173 | 21:21544579 | 91175 | 21:21547758 | 91176 | 21:21554110 | 91178 | 21:21572136 |
| 91189 | 21:21882815 | 91195 | 21:21997010 | 91197 | 21:22018834 | 91201 | 21:22122091 | 91202 | 21:22130826 |
| 91203 | 21:22132465 | 91204 | 21:22137495 | 91205 | 21:22138799 | 91208 | 21:22168604 | 91217 | 21:22354151 |
| 91222 | 21:22473177 | 91223 | 21:22478617 | 91232 | 21:22695822 | 91234 | 21:22803320 | 91240 | 21:22922296 |
| 91244 | 21:22980503 | 91247 | 21:23023062 | 91249 | 21:23081635 | 91250 | 21:23092163 | 91254 | 21:23177845 |
| 91255 | 21:23177853 | 91256 | 21:23181041 | 91257 | 21:23190056 | 91258 | 21:23190653 | 91259 | 21:23195238 |
| 91260 | 21:23211467 | 91284 | 21:24046484 | 91288 | 21:24273010 | 91293 | 21:24375735 | 91347 | 21:26116827 |
| 91361 | 21:26448325 | 91368 | 21:26581951 | 91371 | 21:26637918 | 91383 | 21:26979057 | 91384 | 21:26999182 |
| 91385 | 21:26999251 | 91397 | 21:27503033 | 91398 | 21:27508911 | 91407 | 21:27743344 | 91427 | 21:28338007 |
| 91441 | 21:28917960 | 91443 | 21:28948600 | 91444 | 21:28961889 | 91448 | 21:29052667 | 91461 | 21:29439618 |
| 91465 | 21:29553919 | 91471 | 21:29725150 | 91488 | 21:30205371 | 91489 | 21:30225427 | 91493 | 21:30359230 |
| 91494 | 21:30364775 | 91495 | 21:30364784 | 91497 | 21:30380463 | 91498 | 21:30383546 | 91500 | 21:30415216 |
| 91504 | 21:30548247 | 91507 | 21:30574539 | 91508 | 21:30583259 | 91512 | 21:30655015 | 91513 | 21:30665920 |
| 91514 | 21:30666267 | 91518 | 21:30752337 | 91521 | 21:30836177 | 91525 | 21:31068352 | 91526 | 21:31085819 |
| 91528 | 21:31169053 | 91538 | 21:31453639 | 91539 | 21:31464548 | 91540 | 21:31470928 | 91541 | 21:31485138 |
| 91542 | 21:31493718 | 91543 | 21:31496586 | 91544 | 21:31499460 | 91545 | 21:31522156 | 91546 | 21:31525621 |
| 91547 | 21:31534000 | 91548 | 21:31541459 | 91549 | 21:31545947 | 91550 | 21:31553803 | 91551 | 21:31604982 |
| 91553 | 21:31720553 | 91554 | 21:31725673 | 91555 | 21:31734161 | 91556 | 21:31736971 | 91557 | 21:31754761 |
| 91558 | 21:31758189 | 91559 | 21:31781060 | 91560 | 21:31791229 | 91563 | 21:31955982 | 91574 | 21:32336068 |
| 91575 | 21:32364556 | 91578 | 21:32403515 | 91584 | 21:32692535 | 91585 | 21:32705474 | 91589 | 21:32783989 |
| 91590 | 21:32814280 | 91598 | 21:33110998 | 91602 | 21:33166758 | 91603 | 21:33168203 | 91604 | 21:33169028 |
| 91605 | 21:33170204 | 91606 | 21:33170309 | 91607 | 21:33171324 | 91608 | 21:33171538 | 91610 | 21:33205680 |
| 91611 | 21:33205893 | 91612 | 21:33208660 | 91614 | 21:33215961 | 91615 | 21:33225284 | 91622 | 21:33373120 |
| 91623 | 21:33373124 | 91627 | 21:33449736 | 91628 | 21:33458505 | 91629 | 21:33459344 | 91630 | 21:33461031 |
| 91631 | 21:33461192 | 91632 | 21:33461449 | 91633 | 21:33465757 | 91634 | 21:33465758 | 91635 | 21:33466480 |
| 91636 | 21:33469426 | 91637 | 21:33469555 | 91638 | 21:33469624 | 91639 | 21:33469729 | 91640 | 21:33469770 |
| 91641 | 21:33469982 | 91642 | 21:33483461 | 91643 | 21:33487373 | 91645 | 21:33529381 | 91648 | 21:33547120 |
| 91650 | 21:33571261 | 91652 | 21:33652175 | 91654 | 21:33661162 | 91670 | 21:33947582 | 91672 | 21:33991645 |
| 91675 | 21:34024711 | 91676 | 21:34044738 | 91677 | 21:34049707 | 91680 | 21:34087450 | 91691 | 21:34273652 |
| 91692 | 21:34276064 | 91694 | 21:34296572 | 91703 | 21:34434123 | 91705 | 21:34478879 | 91706 | 21:34487765 |
| 91707 | 21:34490950 | 91712 | 21:34556901 | 91713 | 21:34556925 | 91714 | 21:34557109 | 91715 | 21:34585611 |
| 91718 | 21:34637208 | 91719 | 21:34637260 | 91731 | 21:34938997 | 91732 | 21:34939108 | 91737 | 21:35075467 |
| 91741 | 21:35177228 | 91742 | 21:35177300 | 91752 | 21:35387396 | 91753 | 21:35391211 | 91755 | 21:35545267 |
| 91761 | 21:35621906 | 91787 | 21:36004086 | 91801 | 21:36349981 | 91807 | 21:36467140 | 91844 | 21:37588472 |
| 91855 | 21:37884949 | 91856 | 21:37889952 | 91863 | 21:38352155 | 91866 | 21:38488688 | 91901 | 21:39497698 |
| 91902 | 21:39497796 | 91927 | 21:40117870 | 91928 | 21:40123103 | 91930 | 21:40267167 | 91934 | 21:40321474 |
| 91950 | 21:40777585 | 91955 | 21:41031761 | 91956 | 21:41063587 | 91958 | 21:41103681 | 91959 | 21:41189588 |
| 91960 | 21:41190356 | 91961 | 21:41230647 | 91963 | 21:41268397 | 91968 | 21:41377730 | 91969 | 21:41377811 |
| 91971 | 21:41381520 | 91972 | 21:41381521 | 91973 | 21:41381562 | 91979 | 21:41500659 | 91980 | 21:41500733 |
| 91988 | 21:41683205 | 91995 | 21:41951773 | 91999 | 21:41985647 | 92001 | 21:42021205 | 92005 | 21:42056585 |
| 92006 | 21:42056665 | 92011 | 21:42193358 | 92015 | 21:42263974 | 92025 | 21:42613095 | 92026 | 21:42613704 |
| 92027 | 21:42615428 | 92028 | 21:42632006 | 92036 | 21:42789940 | 92037 | 21:42789959 | 92038 | 21:42794024 |
| 92042 | 21:42894892 | 92043 | 21:42894971 | 92044 | 21:42906559 | 92048 | 21:42991321 | 92060 | 21:43369442 |
| 92073 | 21:43694807 | 92090 | 21:44134802 | 92093 | 21:44170697 | 92094 | 21:44193483 | 92095 | 21:44198391 |
| 92096 | 21:44204274 | 92097 | 21:44204999 | 92098 | 21:44208959 | 92103 | 21:44374543 | 92108 | 21:44464711 |
| 92111 | 21:44510750 | 92112 | 21:44558742 | 92119 | 21:44670112 | 92124 | 21:44711056 | 92125 | 21:44711119 |
| 92128 | 21:44747742 | 92131 | 21:44794407 | 92137 | 21:44874950 | 92146 | 21:45364186 | 92153 | 21:45617906 |
| 92162 | 21:45840926 | 92168 | 21:46169637 | 92177 | 21:46371720 | 92178 | 21:46372978 | 92179 | 21:46375756 |
| 92180 | 21:46377528 | 92181 | 21:46378483 | 92182 | 21:46399567 | 92183 | 21:46411679 | 92207 | 21:47030345 |
| 92209 | 21:47120773 | 92210 | 21:47135961 | 92235 | 21:47300254 | 92252 | 21:47315681 | 92255 | 21:47399940 |
| 92260 | 21:47464301 | 92272 | 21:47936288 | 92293 | 21:48482014 | 92308 | 21:48897167 | 92315 | 21:49088678 |
| 92316 | 21:49165291 | 92317 | 21:49165372 | 92325 | 21:49212043 | 92348 | 21:49756597 | 92349 | 21:49773549 |
| 92414 | 21:51757967 | 92423 | 21:52002988 | 92430 | 21:52140964 | 92438 | 21:52391191 | 92447 | 21:52590908 |
| 92459 | 21:53100162 | 92467 | 21:53274187 | 92468 | 21:53295524 | 92469 | 21:53295551 | 92473 | 21:53382594 |
| 92494 | 21:53955771 | 92511 | 21:54486141 | 92519 | 21:54707788 | 92533 | 21:54895636 | 92534 | 21:54906059 |
| 92535 | 21:54910848 | 92536 | 21:54911588 | 92537 | 21:54911598 | 92538 | 21:54919544 | 92540 | 21:54956256 |
| 92544 | 21:55027058 | 92549 | 21:55139938 | 92551 | 21:55157787 | 92553 | 21:55268142 | 92555 | 21:55276126 |
| 92559 | 21:55336100 | 92567 | 21:55534889 | 92600 | 21:56030046 | 92609 | 21:56102239 | 92611 | 21:56151393 |
| 92612 | 21:56165597 | 92613 | 21:56174882 | 92614 | 21:56184939 | 92616 | 21:56194199 | 92617 | 21:56195714 |
| 92618 | 21:56211969 | 92625 | 21:56552387 | 92627 | 21:56570919 | 92637 | 21:56804066 | 92689 | 21:57070861 |
| 92690 | 21:57070865 | 92713 | 21:57163461 | 92714 | 21:57163519 | 92730 | 21:57357698 | 92737 | 21:57514823 |
| 92740 | 21:57650013 | 92744 | 21:57789826 | 92745 | 21:57815184 | 92746 | 21:57841247 | 92752 | 21:58086488 |
| 92760 | 21:58328406 | 92761 | 21:58335169 | 92762 | 21:58348854 | 92765 | 21:58457450 | 92769 | 21:58542608 |
| 92777 | 21:58649278 | 92778 | 21:58649996 | 92779 | 21:58650822 | 92780 | 21:58651655 | 92781 | 21:58651789 |
| 92782 | 21:58653170 | 92783 | 21:58653759 | 92784 | 21:58653796 | 92785 | 21:58654257 | 92786 | 21:58657343 |
| 92787 | 21:58730952 | 92792 | 21:58844584 | 92794 | 21:58897764 | 92812 | 21:59280395 | 92813 | 21:59290376 |
| 92816 | 21:59410654 | 92817 | 21:59450928 | 92821 | 21:59494769 | 92838 | 21:59798491 | 92846 | 21:59885566 |
| 92847 | 21:59892560 | 92854 | 21:59967817 | 92857 | 21:59995456 | 92858 | 21:60034051 | 92860 | 21:60070408 |
| 92864 | 21:60128571 | 92867 | 21:60147487 | 92870 | 21:60222745 | 92872 | 21:60258085 | 92875 | 21:60285746 |
| 92878 | 21:60309341 | 92879 | 21:60318597 | 92880 | 21:60364407 | 92887 | 21:60531623 | 92888 | 21:60531761 |
| 92914 | 21:60963159 | 92929 | 21:61332759 | 92935 | 21:61617246 | 92936 | 21:61637616 | 92937 | 21:61639799 |
| 92938 | 21:61640498 | 92957 | 21:62025836 | 92973 | 21:62390233 | 93001 | 21:63134292 | 93003 | 21:63222235 |
| 93004 | 21:63258242 | 93010 | 21:63480195 | 93017 | 21:63603801 | 93024 | 21:63831078 | 93029 | 21:63935578 |
| 93046 | 21:64333571 | 93050 | 21:64428779 | 93051 | 21:64450577 | 93052 | 21:64452975 | 93065 | 21:64633547 |
| 93070 | 21:64699689 | 93077 | 21:64841812 | 93078 | 21:64841843 | 93079 | 21:64841865 | 93080 | 21:64850271 |
| 93084 | 21:64922535 | 93086 | 21:64988072 | 93088 | 21:65014550 | 93089 | 21:65027672 | 93090 | 21:65029870 |
| 93091 | 21:65051618 | 93092 | 21:65052179 | 93093 | 21:65052717 | 93094 | 21:65054313 | 93095 | 21:65055762 |
| 93096 | 21:65056739 | 93097 | 21:65060441 | 93098 | 21:65061116 | 93099 | 21:65067323 | 93100 | 21:65073410 |
| 93101 | 21:65073892 | 93102 | 21:65075325 | 93103 | 21:65079623 | 93104 | 21:65080143 | 93105 | 21:65082481 |
| 93114 | 21:65232598 | 93167 | 21:66311776 | 93183 | 21:66723461 | 93189 | 21:66870945 | 93190 | 21:66929334 |
| 93191 | 21:66932880 | 93192 | 21:66934300 | 93193 | 21:66936241 | 93194 | 21:66938188 | 93195 | 21:66942278 |
| 93197 | 21:66945357 | 93204 | 21:67094952 | 93208 | 21:67149363 | 93215 | 21:67449798 | 93221 | 21:67552612 |
| 93231 | 21:67865058 | 93232 | 21:67876373 | 93240 | 21:67965438 | 93251 | 21:68196501 | 93252 | 21:68202138 |
| 93253 | 21:68202185 | 93255 | 21:68230479 | 93258 | 21:68286931 | 93260 | 21:68335052 | 93268 | 21:68506930 |
| 93270 | 21:68519125 | 93271 | 21:68522170 | 93272 | 21:68529965 | 93273 | 21:68530561 | 93274 | 21:68530572 |
| 93276 | 21:68537086 | 93280 | 21:68668451 | 93294 | 21:69004265 | 93301 | 21:69165607 | 93304 | 21:69229046 |
| 93308 | 21:69352283 | 93309 | 21:69352557 | 93310 | 21:69353990 | 93312 | 21:69409067 | 93314 | 21:69440442 |
| 93315 | 21:69440464 | 93316 | 21:69440466 | 93317 | 21:69442039 | 93320 | 21:69557781 | 93321 | 21:69574694 |
| 93322 | 21:69582716 | 93324 | 21:69629308 | 93325 | 21:69642755 | 93326 | 21:69660286 | 93331 | 21:69725960 |
| 93332 | 21:69764707 | 93333 | 21:69765916 | 93334 | 21:69766081 | 93335 | 21:69770916 | 93345 | 22:378758 |
| 93353 | 22:651171 | 93363 | 22:912338 | 93366 | 22:940171 | 93367 | 22:940370 | 93395 | 22:1560877 |
| 93401 | 22:1738773 | 93402 | 22:1738804 | 93403 | 22:1738805 | 93404 | 22:1746536 | 93414 | 22:1927654 |
| 93432 | 22:2329624 | 93449 | 22:2751520 | 93450 | 22:2752726 | 93456 | 22:2877258 | 93457 | 22:2892709 |
| 93465 | 22:3056974 | 93471 | 22:3197001 | 93473 | 22:3220010 | 93475 | 22:3269948 | 93482 | 22:3359434 |
| 93493 | 22:3596980 | 93499 | 22:3798423 | 93501 | 22:3834731 | 93504 | 22:3914101 | 93506 | 22:3960951 |
| 93529 | 22:4497784 | 93540 | 22:4729907 | 93546 | 22:4842711 | 93551 | 22:4922676 | 93556 | 22:5030837 |
| 93559 | 22:5120220 | 93561 | 22:5160439 | 93562 | 22:5165375 | 93564 | 22:5179197 | 93575 | 22:5341524 |
| 93576 | 22:5345473 | 93577 | 22:5391435 | 93584 | 22:5485152 | 93595 | 22:5647094 | 93597 | 22:5693798 |
| 93598 | 22:5721636 | 93600 | 22:5764445 | 93605 | 22:5883723 | 93612 | 22:5950788 | 93616 | 22:6008417 |
| 93617 | 22:6017167 | 93618 | 22:6052161 | 93622 | 22:6199633 | 93627 | 22:6252438 | 93630 | 22:6293377 |
| 93636 | 22:6370667 | 93643 | 22:6488129 | 93645 | 22:6498701 | 93657 | 22:6784495 | 93660 | 22:6839952 |
| 93671 | 22:6996393 | 93685 | 22:7428239 | 93686 | 22:7434501 | 93697 | 22:7651542 | 93702 | 22:7841139 |
| 93703 | 22:7908696 | 93723 | 22:8411667 | 93735 | 22:8761701 | 93772 | 22:9753217 | 93774 | 22:9795347 |
| 93783 | 22:10006268 | 93784 | 22:10006273 | 93785 | 22:10006376 | 93787 | 22:10035911 | 93789 | 22:10081106 |
| 93790 | 22:10081118 | 93791 | 22:10081165 | 93792 | 22:10081187 | 93793 | 22:10081208 | 93794 | 22:10081215 |
| 93795 | 22:10081251 | 93796 | 22:10081295 | 93801 | 22:10172170 | 93804 | 22:10236047 | 93809 | 22:10290595 |
| 93810 | 22:10345715 | 93812 | 22:10401777 | 93836 | 22:11003544 | 93841 | 22:11122201 | 93850 | 22:11391522 |
| 93858 | 22:11578648 | 93860 | 22:11718954 | 93861 | 22:11720712 | 93865 | 22:11758613 | 93867 | 22:11831403 |
| 93868 | 22:11832624 | 93869 | 22:11889790 | 93870 | 22:11889853 | 93871 | 22:11890006 | 93872 | 22:11890127 |
| 93878 | 22:11973585 | 93879 | 22:11977784 | 93880 | 22:11977859 | 93881 | 22:11978609 | 93882 | 22:11978855 |
| 93883 | 22:11978903 | 93886 | 22:12019884 | 93892 | 22:12195260 | 93897 | 22:12303365 | 93900 | 22:12377148 |
| 93901 | 22:12430483 | 93902 | 22:12436371 | 93904 | 22:12451350 | 93907 | 22:12503200 | 93908 | 22:12506458 |
| 93917 | 22:12662972 | 93921 | 22:12724433 | 93930 | 22:12923416 | 93936 | 22:13091338 | 93943 | 22:13359849 |
| 93951 | 22:13559915 | 93952 | 22:13582865 | 93969 | 22:14142677 | 93970 | 22:14145371 | 93973 | 22:14181865 |
| 93976 | 22:14213464 | 93978 | 22:14312375 | 93984 | 22:14511531 | 93985 | 22:14512264 | 93986 | 22:14516780 |
| 93987 | 22:14516787 | 93988 | 22:14518692 | 93989 | 22:14519180 | 94006 | 22:15072933 | 94008 | 22:15123459 |
| 94019 | 22:15471657 | 94022 | 22:15504111 | 94027 | 22:15605731 | 94051 | 22:16322403 | 94052 | 22:16322451 |
| 94053 | 22:16322488 | 94054 | 22:16353524 | 94062 | 22:16546381 | 94063 | 22:16548801 | 94064 | 22:16561111 |
| 94065 | 22:16567113 | 94066 | 22:16567586 | 94067 | 22:16568933 | 94073 | 22:16666585 | 94077 | 22:16725586 |
| 94078 | 22:16728120 | 94080 | 22:16741450 | 94082 | 22:16757417 | 94083 | 22:16773003 | 94084 | 22:16802200 |
| 94085 | 22:16804567 | 94100 | 22:17418863 | 94113 | 22:17718315 | 94116 | 22:17791469 | 94123 | 22:17937879 |
| 94125 | 22:17953330 | 94130 | 22:18035176 | 94132 | 22:18061536 | 94151 | 22:18541264 | 94156 | 22:18614346 |
| 94160 | 22:18696586 | 94164 | 22:18832117 | 94175 | 22:19093982 | 94177 | 22:19212118 | 94185 | 22:19376422 |
| 94215 | 22:20299573 | 94221 | 22:20368774 | 94234 | 22:20830719 | 94259 | 22:21264256 | 94260 | 22:21281505 |
| 94262 | 22:21343700 | 94270 | 22:21553615 | 94272 | 22:21581948 | 94275 | 22:21638280 | 94282 | 22:21687964 |
| 94284 | 22:21691311 | 94285 | 22:21691369 | 94286 | 22:21696971 | 94287 | 22:21697320 | 94289 | 22:21780680 |
| 94295 | 22:21936689 | 94298 | 22:22018100 | 94301 | 22:22096896 | 94302 | 22:22130081 | 94304 | 22:22207036 |
| 94305 | 22:22280513 | 94306 | 22:22280554 | 94312 | 22:22511637 | 94321 | 22:22847965 | 94325 | 22:22910885 |
| 94328 | 22:22978479 | 94390 | 22:24919962 | 94419 | 22:25887179 | 94426 | 22:26004558 | 94427 | 22:26004600 |
| 94428 | 22:26006402 | 94429 | 22:26028973 | 94431 | 22:26048198 | 94432 | 22:26048256 | 94433 | 22:26055574 |
| 94437 | 22:26092272 | 94438 | 22:26092293 | 94442 | 22:26179382 | 94443 | 22:26199906 | 94446 | 22:26342475 |
| 94474 | 22:27220605 | 94480 | 22:27400091 | 94490 | 22:27635934 | 94507 | 22:28135962 | 94512 | 22:28188835 |
| 94514 | 22:28211359 | 94518 | 22:28297855 | 94523 | 22:28404142 | 94524 | 22:28404219 | 94529 | 22:28481900 |
| 94531 | 22:28515749 | 94533 | 22:28587264 | 94534 | 22:28588309 | 94539 | 22:28733810 | 94541 | 22:28784852 |
| 94542 | 22:28784985 | 94543 | 22:28792168 | 94545 | 22:28835841 | 94546 | 22:28835907 | 94547 | 22:28835973 |
| 94548 | 22:28840124 | 94549 | 22:28840129 | 94550 | 22:28843084 | 94551 | 22:28844382 | 94552 | 22:28845975 |
| 94554 | 22:28862947 | 94555 | 22:28938955 | 94557 | 22:29005992 | 94566 | 22:29299265 | 94581 | 22:29705813 |
| 94582 | 22:29709388 | 94583 | 22:29712615 | 94604 | 22:30274003 | 94607 | 22:30417109 | 94608 | 22:30436771 |
| 94610 | 22:30515471 | 94624 | 22:30808716 | 94638 | 22:31133292 | 94639 | 22:31135175 | 94640 | 22:31141034 |
| 94661 | 22:31835374 | 94662 | 22:31861512 | 94663 | 22:31891766 | 94666 | 22:32022974 | 94673 | 22:32156119 |
| 94674 | 22:32186880 | 94675 | 22:32233824 | 94676 | 22:32246231 | 94679 | 22:32399020 | 94685 | 22:32572065 |
| 94688 | 22:32679434 | 94698 | 22:32982493 | 94700 | 22:32993742 | 94701 | 22:33017003 | 94702 | 22:33053278 |
| 94708 | 22:33142515 | 94728 | 22:33935530 | 94729 | 22:33944757 | 94733 | 22:33983138 | 94768 | 22:34968813 |
| 94771 | 22:35012254 | 94774 | 22:35039773 | 94781 | 22:35173959 | 94791 | 22:35329495 | 94793 | 22:35423874 |
| 94796 | 22:35459751 | 94797 | 22:35460784 | 94799 | 22:35523854 | 94810 | 22:35736939 | 94812 | 22:35774994 |
| 94815 | 22:35862159 | 94816 | 22:35890192 | 94817 | 22:35901222 | 94826 | 22:36031535 | 94837 | 22:36381197 |
| 94844 | 22:36496828 | 94846 | 22:36551821 | 94848 | 22:36609940 | 94856 | 22:36967669 | 94865 | 22:37191405 |
| 94866 | 22:37210076 | 94872 | 22:37384626 | 94873 | 22:37391008 | 94876 | 22:37473284 | 94883 | 22:37617112 |
| 94884 | 22:37620082 | 94885 | 22:37622201 | 94886 | 22:37624592 | 94887 | 22:37627226 | 94888 | 22:37628993 |
| 94889 | 22:37635996 | 94890 | 22:37636848 | 94891 | 22:37637997 | 94892 | 22:37640217 | 94893 | 22:37653222 |
| 94917 | 22:38367562 | 94919 | 22:38384223 | 94924 | 22:38493420 | 94928 | 22:38548453 | 94934 | 22:38617876 |
| 94944 | 22:38801073 | 94955 | 22:39102590 | 94963 | 22:39227562 | 94964 | 22:39234430 | 94965 | 22:39240885 |
| 94977 | 22:39399375 | 94978 | 22:39399383 | 94996 | 22:39664313 | 95001 | 22:39736141 | 95002 | 22:39736166 |
| 95003 | 22:39742270 | 95005 | 22:39764001 | 95007 | 22:39806230 | 95022 | 22:40159960 | 95028 | 22:40412439 |
| 95030 | 22:40474842 | 95031 | 22:40497320 | 95033 | 22:40497948 | 95036 | 22:40553964 | 95043 | 22:40698664 |
| 95044 | 22:40699122 | 95046 | 22:40709407 | 95064 | 22:41331703 | 95077 | 22:41673525 | 95078 | 22:41697991 |
| 95104 | 22:42421167 | 95108 | 22:42552768 | 95118 | 22:42753833 | 95120 | 22:42860068 | 95131 | 22:43070171 |
| 95133 | 22:43103372 | 95135 | 22:43152197 | 95137 | 22:43171695 | 95139 | 22:43185881 | 95142 | 22:43216398 |
| 95145 | 22:43241133 | 95148 | 22:43280264 | 95151 | 22:43327456 | 95152 | 22:43328177 | 95158 | 22:43532708 |
| 95162 | 22:43617671 | 95170 | 22:43831970 | 95172 | 22:43860815 | 95190 | 22:44240079 | 95191 | 22:44242443 |
| 95195 | 22:44303591 | 95212 | 22:44704423 | 95217 | 22:44800260 | 95225 | 22:45101725 | 95232 | 22:45343274 |
| 95245 | 22:45656061 | 95247 | 22:45691255 | 95252 | 22:45783940 | 95255 | 22:45826883 | 95270 | 22:46040006 |
| 95273 | 22:46071530 | 95291 | 22:46619809 | 95296 | 22:46740639 | 95298 | 22:46748886 | 95311 | 22:46981263 |
| 95312 | 22:46981314 | 95315 | 22:47033448 | 95318 | 22:47057561 | 95331 | 22:47235742 | 95333 | 22:47261646 |
| 95337 | 22:47402038 | 95342 | 22:47467741 | 95349 | 22:47617100 | 95352 | 22:47677699 | 95356 | 22:47817718 |
| 95361 | 22:48000435 | 95364 | 22:48019220 | 95369 | 22:48066613 | 95370 | 22:48091175 | 95371 | 22:48107831 |
| 95372 | 22:48170389 | 95373 | 22:48171831 | 95380 | 22:48435818 | 95381 | 22:48449692 | 95386 | 22:48687235 |
| 95388 | 22:48709706 | 95391 | 22:48761536 | 95395 | 22:48961009 | 95396 | 22:48979936 | 95397 | 22:48988156 |
| 95398 | 22:49025901 | 95399 | 22:49040313 | 95400 | 22:49041656 | 95405 | 22:49152003 | 95407 | 22:49218903 |
| 95411 | 22:49396397 | 95412 | 22:49411257 | 95413 | 22:49416701 | 95417 | 22:49475649 | 95422 | 22:49751795 |
| 95427 | 22:49845416 | 95428 | 22:49855390 | 95429 | 22:49862171 | 95430 | 22:49874759 | 95437 | 22:50031711 |
| 95443 | 22:50110984 | 95444 | 22:50127865 | 95446 | 22:50174136 | 95448 | 22:50267951 | 95453 | 22:50398849 |
| 95463 | 22:50615575 | 95464 | 22:50626022 | 95467 | 22:50669330 | 95468 | 22:50669331 | 95471 | 22:50732557 |
| 95472 | 22:50752636 | 95475 | 22:50849444 | 95488 | 22:51284290 | 95490 | 22:51317516 | 95495 | 22:51400864 |
| 95502 | 22:51573666 | 95509 | 22:51800322 | 95517 | 22:51985899 | 95518 | 22:51994258 | 95521 | 22:52090420 |
| 95522 | 22:52090421 | 95523 | 22:52090571 | 95526 | 22:52113515 | 95528 | 22:52142954 | 95535 | 22:52398536 |
| 95536 | 22:52409661 | 95545 | 22:52642109 | 95547 | 22:52689484 | 95555 | 22:52876456 | 95559 | 22:52889478 |
| 95562 | 22:52940887 | 95567 | 22:52998347 | 95568 | 22:52998797 | 95569 | 22:52998800 | 95570 | 22:52999211 |
| 95571 | 22:52999397 | 95573 | 22:53017868 | 95586 | 22:53333639 | 95595 | 22:53587524 | 95596 | 22:53630193 |
| 95597 | 22:53632109 | 95599 | 22:53667747 | 95601 | 22:53701852 | 95603 | 22:53750298 | 95604 | 22:53771417 |
| 95608 | 22:53814947 | 95609 | 22:53815430 | 95610 | 22:53823185 | 95617 | 22:53952061 | 95625 | 22:54050342 |
| 95626 | 22:54084951 | 95634 | 22:54271229 | 95636 | 22:54325220 | 95637 | 22:54325294 | 95638 | 22:54326413 |
| 95639 | 22:54326417 | 95640 | 22:54326525 | 95652 | 22:54582223 | 95654 | 22:54586616 | 95655 | 22:54590257 |
| 95656 | 22:54591674 | 95657 | 22:54601062 | 95659 | 22:54619999 | 95660 | 22:54656963 | 95661 | 22:54657085 |
| 95681 | 22:55222425 | 95682 | 22:55249217 | 95684 | 22:55290303 | 95685 | 22:55290330 | 95687 | 22:55290410 |
| 95688 | 22:55290479 | 95689 | 22:55290914 | 95690 | 22:55291002 | 95691 | 22:55291168 | 95692 | 22:55291236 |
| 95693 | 22:55291381 | 95694 | 22:55291382 | 95697 | 22:55338384 | 95699 | 22:55379431 | 95700 | 22:55413027 |
| 95705 | 22:55446332 | 95706 | 22:55455982 | 95709 | 22:55483225 | 95713 | 22:55579849 | 95725 | 22:55804900 |
| 95728 | 22:55847268 | 95733 | 22:55887174 | 95734 | 22:55887346 | 95735 | 22:55887394 | 95736 | 22:55887397 |
| 95737 | 22:55887398 | 95738 | 22:55887409 | 95739 | 22:55887425 | 95740 | 22:55887575 | 95741 | 22:55888374 |
| 95746 | 22:55952909 | 95759 | 22:56301696 | 95761 | 22:56325207 | 95762 | 22:56325210 | 95763 | 22:56325225 |
| 95764 | 22:56325235 | 95765 | 22:56325272 | 95766 | 22:56325600 | 95767 | 22:56325606 | 95769 | 22:56325745 |
| 95770 | 22:56325754 | 95771 | 22:56326527 | 95772 | 22:56329687 | 95773 | 22:56329721 | 95774 | 22:56332166 |
| 95775 | 22:56340185 | 95777 | 22:56377918 | 95778 | 22:56388281 | 95780 | 22:56396437 | 95791 | 22:56484402 |
| 95792 | 22:56513273 | 95797 | 22:56546272 | 95800 | 22:56616418 | 95801 | 22:56632616 | 95822 | 22:57017818 |
| 95823 | 22:57017940 | 95825 | 22:57074332 | 95827 | 22:57132556 | 95830 | 22:57180726 | 95831 | 22:57198542 |
| 95832 | 22:57228439 | 95833 | 22:57257734 | 95834 | 22:57268915 | 95840 | 22:57427960 | 95841 | 22:57434193 |
| 95843 | 22:57453058 | 95846 | 22:57478438 | 95847 | 22:57511375 | 95851 | 22:57532676 | 95855 | 22:57579073 |
| 95857 | 22:57629519 | 95858 | 22:57638240 | 95869 | 22:57759457 | 95870 | 22:57764469 | 95871 | 22:57766664 |
| 95872 | 22:57792445 | 95890 | 22:58069483 | 95891 | 22:58075933 | 95892 | 22:58086195 | 95905 | 22:58427446 |
| 95907 | 22:58446882 | 95911 | 22:58506677 | 95925 | 22:58804900 | 95938 | 22:59105276 | 95939 | 22:59111787 |
| 95945 | 22:59165452 | 95947 | 22:59174986 | 95950 | 22:59237930 | 95961 | 22:59518872 | 95966 | 22:59625926 |
| 95978 | 22:59729840 | 95982 | 22:59783092 | 95983 | 22:59831717 | 95989 | 22:59928044 | 96005 | 22:60263301 |
| 96007 | 22:60314302 | 96008 | 22:60344480 | 96009 | 22:60369311 | 96013 | 22:60433161 | 96014 | 22:60450289 |
| 96015 | 22:60450822 | 96016 | 22:60450826 | 96021 | 22:60583428 | 96024 | 22:60664148 | 96028 | 23:98715 |
| 96048 | 23:908722 | 96059 | 23:1233960 | 96067 | 23:1413088 | 96081 | 23:1806494 | 96089 | 23:2027290 |
| 96091 | 23:2085668 | 96102 | 23:2295458 | 96103 | 23:2327306 | 96109 | 23:2490639 | 96115 | 23:2633185 |
| 96119 | 23:2653923 | 96121 | 23:2672734 | 96122 | 23:2681218 | 96131 | 23:2932961 | 96132 | 23:2932963 |
| 96133 | 23:2933026 | 96134 | 23:2933089 | 96135 | 23:2933119 | 96136 | 23:2943350 | 96137 | 23:2944259 |
| 96138 | 23:2945377 | 96147 | 23:3182108 | 96150 | 23:3278552 | 96152 | 23:3363897 | 96154 | 23:3397926 |
| 96155 | 23:3401808 | 96156 | 23:3422753 | 96157 | 23:3441692 | 96158 | 23:3451376 | 96167 | 23:3623220 |
| 96169 | 23:3649638 | 96172 | 23:3661124 | 96173 | 23:3691486 | 96174 | 23:3694848 | 96187 | 23:3953291 |
| 96195 | 23:4055037 | 96207 | 23:4270316 | 96211 | 23:4397147 | 96257 | 23:5221777 | 96270 | 23:5551234 |
| 96273 | 23:5596536 | 96274 | 23:5598882 | 96275 | 23:5598939 | 96276 | 23:5598954 | 96277 | 23:5598967 |
| 96291 | 23:6007499 | 96302 | 23:6313109 | 96315 | 23:6596002 | 96316 | 23:6596059 | 96319 | 23:6615760 |
| 96320 | 23:6619709 | 96321 | 23:6619777 | 96334 | 23:6834687 | 96343 | 23:7086052 | 96348 | 23:7176749 |
| 96349 | 23:7185030 | 96351 | 23:7220926 | 96354 | 23:7287024 | 96362 | 23:7470856 | 96366 | 23:7588787 |
| 96367 | 23:7590703 | 96368 | 23:7604788 | 96369 | 23:7627114 | 96370 | 23:7627927 | 96385 | 23:8019039 |
| 96386 | 23:8021311 | 96388 | 23:8043634 | 96394 | 23:8257417 | 96395 | 23:8265499 | 96402 | 23:8382452 |
| 96403 | 23:8386569 | 96405 | 23:8392718 | 96406 | 23:8394550 | 96407 | 23:8403739 | 96409 | 23:8430336 |
| 96410 | 23:8437470 | 96411 | 23:8444830 | 96412 | 23:8458254 | 96423 | 23:8693541 | 96428 | 23:8861332 |
| 96429 | 23:8861347 | 96435 | 23:8977884 | 96438 | 23:9024159 | 96439 | 23:9024206 | 96440 | 23:9024246 |
| 96447 | 23:9163290 | 96448 | 23:9164368 | 96452 | 23:9341201 | 96457 | 23:9496576 | 96460 | 23:9564042 |
| 96461 | 23:9565542 | 96462 | 23:9583880 | 96464 | 23:9685892 | 96466 | 23:9745811 | 96467 | 23:9767204 |
| 96476 | 23:10029783 | 96483 | 23:10140851 | 96485 | 23:10256230 | 96486 | 23:10257763 | 96487 | 23:10257827 |
| 96488 | 23:10258971 | 96489 | 23:10259318 | 96490 | 23:10259504 | 96492 | 23:10261010 | 96493 | 23:10262012 |
| 96494 | 23:10262085 | 96495 | 23:10262281 | 96496 | 23:10262341 | 96497 | 23:10262386 | 96498 | 23:10262523 |
| 96501 | 23:10328877 | 96506 | 23:10459370 | 96514 | 23:10615240 | 96515 | 23:10638380 | 96519 | 23:10710493 |
| 96523 | 23:10803832 | 96524 | 23:10870495 | 96527 | 23:10883412 | 96528 | 23:10883649 | 96529 | 23:10890338 |
| 96530 | 23:10913209 | 96532 | 23:10921995 | 96533 | 23:10922014 | 96538 | 23:11106125 | 96554 | 23:11574894 |
| 96555 | 23:11592749 | 96556 | 23:11599745 | 96564 | 23:11737588 | 96566 | 23:11785803 | 96573 | 23:11925495 |
| 96575 | 23:11950761 | 96576 | 23:11956619 | 96586 | 23:12073751 | 96591 | 23:12208427 | 96594 | 23:12235079 |
| 96603 | 23:12561054 | 96604 | 23:12561114 | 96605 | 23:12561116 | 96606 | 23:12561252 | 96607 | 23:12561302 |
| 96608 | 23:12564591 | 96609 | 23:12566537 | 96625 | 23:12975952 | 96626 | 23:13002407 | 96631 | 23:13062813 |
| 96632 | 23:13062870 | 96633 | 23:13063271 | 96634 | 23:13065594 | 96635 | 23:13065660 | 96636 | 23:13066259 |
| 96637 | 23:13073019 | 96638 | 23:13083039 | 96639 | 23:13096550 | 96640 | 23:13119734 | 96645 | 23:13155594 |
| 96650 | 23:13360736 | 96668 | 23:13797670 | 96669 | 23:13803074 | 96673 | 23:13844075 | 96682 | 23:13890619 |
| 96683 | 23:13913723 | 96684 | 23:13917180 | 96711 | 23:14503938 | 96713 | 23:14550205 | 96726 | 23:14801418 |
| 96736 | 23:14938004 | 96740 | 23:14977766 | 96744 | 23:15085370 | 96746 | 23:15116016 | 96747 | 23:15116055 |
| 96748 | 23:15118596 | 96749 | 23:15119248 | 96750 | 23:15119542 | 96751 | 23:15119571 | 96752 | 23:15119572 |
| 96753 | 23:15119619 | 96754 | 23:15135050 | 96761 | 23:15271919 | 96763 | 23:15283396 | 96766 | 23:15345255 |
| 96773 | 23:15533652 | 96778 | 23:15581761 | 96795 | 23:15759615 | 96801 | 23:15883997 | 96803 | 23:15932643 |
| 96813 | 23:16272106 | 96820 | 23:16456191 | 96822 | 23:16466008 | 96823 | 23:16479564 | 96829 | 23:16661447 |
| 96831 | 23:16686014 | 96832 | 23:16714066 | 96835 | 23:16792993 | 96836 | 23:16801536 | 96843 | 23:17062554 |
| 96859 | 23:17556957 | 96868 | 23:17708671 | 96869 | 23:17718607 | 96870 | 23:17737639 | 96890 | 23:18161337 |
| 96891 | 23:18200714 | 96894 | 23:18216510 | 96935 | 23:19331450 | 96936 | 23:19366522 | 96939 | 23:19415329 |
| 96966 | 23:19751742 | 96970 | 23:19767163 | 96974 | 23:19826206 | 96976 | 23:19925315 | 96982 | 23:19970969 |
| 96985 | 23:20042631 | 96986 | 23:20052143 | 96989 | 23:20102219 | 96991 | 23:20121033 | 96997 | 23:20203703 |
| 96998 | 23:20223241 | 97007 | 23:20409619 | 97008 | 23:20409671 | 97009 | 23:20409761 | 97024 | 23:20698756 |
| 97025 | 23:20786968 | 97028 | 23:20835335 | 97029 | 23:20835446 | 97045 | 23:21265221 | 97050 | 23:21344891 |
| 97056 | 23:21453135 | 97057 | 23:21453252 | 97066 | 23:21870117 | 97085 | 23:22255326 | 97088 | 23:22364773 |
| 97113 | 23:23196063 | 97126 | 23:23576448 | 97135 | 23:23755239 | 97136 | 23:23762207 | 97138 | 23:23782323 |
| 97148 | 23:23940335 | 97149 | 23:23968151 | 97156 | 23:24092418 | 97164 | 23:24300414 | 97165 | 23:24303099 |
| 97167 | 23:24343369 | 97168 | 23:24345237 | 97169 | 23:24352270 | 97173 | 23:24416257 | 97174 | 23:24423273 |
| 97179 | 23:24498401 | 97180 | 23:24505272 | 97186 | 23:24683101 | 97187 | 23:24685752 | 97188 | 23:24686739 |
| 97189 | 23:24689461 | 97190 | 23:24692653 | 97191 | 23:24699153 | 97192 | 23:24700095 | 97193 | 23:24701041 |
| 97194 | 23:24702797 | 97195 | 23:24703647 | 97196 | 23:24704414 | 97197 | 23:24705785 | 97198 | 23:24709728 |
| 97200 | 23:24717517 | 97201 | 23:24718990 | 97202 | 23:24723139 | 97203 | 23:24727842 | 97204 | 23:24729644 |
| 97205 | 23:24730615 | 97206 | 23:24732566 | 97207 | 23:24733786 | 97208 | 23:24736986 | 97209 | 23:24739399 |
| 97210 | 23:24744157 | 97213 | 23:24790792 | 97214 | 23:24794179 | 97216 | 23:24799157 | 97217 | 23:24800192 |
| 97218 | 23:24823698 | 97219 | 23:24823737 | 97224 | 23:24880628 | 97226 | 23:24916092 | 97228 | 23:24934787 |
| 97229 | 23:24936283 | 97230 | 23:24939717 | 97232 | 23:24975703 | 97240 | 23:25055772 | 97243 | 23:25088466 |
| 97244 | 23:25089919 | 97245 | 23:25102180 | 97247 | 23:25129357 | 97248 | 23:25130486 | 97249 | 23:25143923 |
| 97250 | 23:25145004 | 97251 | 23:25146146 | 97252 | 23:25156088 | 97253 | 23:25164433 | 97254 | 23:25166831 |
| 97257 | 23:25198505 | 97258 | 23:25199834 | 97259 | 23:25200752 | 97260 | 23:25201563 | 97264 | 23:25222382 |
| 97265 | 23:25235072 | 97267 | 23:25235958 | 97269 | 23:25250609 | 97271 | 23:25276948 | 97272 | 23:25283721 |
| 97273 | 23:25287389 | 97275 | 23:25323788 | 97279 | 23:25354558 | 97280 | 23:25354656 | 97282 | 23:25372486 |
| 97286 | 23:25406424 | 97287 | 23:25406813 | 97289 | 23:25433764 | 97292 | 23:25467868 | 97296 | 23:25560755 |
| 97298 | 23:25617278 | 97299 | 23:25674209 | 97300 | 23:25680001 | 97301 | 23:25689132 | 97302 | 23:25690596 |
| 97303 | 23:25692139 | 97304 | 23:25693451 | 97306 | 23:25715869 | 97308 | 23:25730240 | 97309 | 23:25730292 |
| 97310 | 23:25730339 | 97311 | 23:25750106 | 97312 | 23:25755389 | 97313 | 23:25758772 | 97314 | 23:25759863 |
| 97315 | 23:25764182 | 97316 | 23:25767576 | 97317 | 23:25780173 | 97318 | 23:25781822 | 97319 | 23:25786244 |
| 97320 | 23:25788400 | 97322 | 23:25818270 | 97323 | 23:25819563 | 97324 | 23:25819612 | 97325 | 23:25819634 |
| 97327 | 23:25825333 | 97328 | 23:25827861 | 97330 | 23:25834509 | 97331 | 23:25840572 | 97332 | 23:25842401 |
| 97335 | 23:25902950 | 97352 | 23:26938031 | 97353 | 23:26943744 | 97354 | 23:26947613 | 97360 | 23:27113071 |
| 97361 | 23:27113077 | 97362 | 23:27113082 | 97363 | 23:27113092 | 97364 | 23:27113093 | 97366 | 23:27139603 |
| 97367 | 23:27139619 | 97368 | 23:27139643 | 97369 | 23:27141360 | 97370 | 23:27144341 | 97372 | 23:27175642 |
| 97373 | 23:27176260 | 97379 | 23:27233819 | 97381 | 23:27306574 | 97382 | 23:27317678 | 97383 | 23:27325182 |
| 97384 | 23:27348931 | 97385 | 23:27355151 | 97386 | 23:27356345 | 97387 | 23:27360333 | 97388 | 23:27361033 |
| 97389 | 23:27361247 | 97390 | 23:27361255 | 97392 | 23:27385789 | 97393 | 23:27397427 | 97394 | 23:27397904 |
| 97395 | 23:27398878 | 97402 | 23:27492296 | 97403 | 23:27493205 | 97404 | 23:27494111 | 97406 | 23:27497189 |
| 97407 | 23:27497222 | 97408 | 23:27497848 | 97409 | 23:27497875 | 97410 | 23:27497880 | 97411 | 23:27499647 |
| 97412 | 23:27514878 | 97413 | 23:27516482 | 97414 | 23:27522712 | 97415 | 23:27528967 | 97417 | 23:27538128 |
| 97419 | 23:27545924 | 97421 | 23:27601917 | 97425 | 23:27635451 | 97426 | 23:27643343 | 97428 | 23:27657685 |
| 97431 | 23:27701786 | 97432 | 23:27727500 | 97435 | 23:27774805 | 97436 | 23:27801348 | 97437 | 23:27815800 |
| 97438 | 23:27824468 | 97440 | 23:27883512 | 97441 | 23:27910429 | 97442 | 23:27976925 | 97443 | 23:27978843 |
| 97445 | 23:27985061 | 97446 | 23:27988153 | 97447 | 23:27988939 | 97449 | 23:27989644 | 97452 | 23:28001628 |
| 97453 | 23:28002243 | 97454 | 23:28003650 | 97455 | 23:28006280 | 97456 | 23:28016941 | 97457 | 23:28023809 |
| 97458 | 23:28024215 | 97459 | 23:28024237 | 97460 | 23:28029876 | 97461 | 23:28030196 | 97462 | 23:28040546 |
| 97463 | 23:28054764 | 97464 | 23:28055141 | 97465 | 23:28055144 | 97466 | 23:28055147 | 97467 | 23:28055154 |
| 97468 | 23:28055203 | 97469 | 23:28055205 | 97470 | 23:28056174 | 97471 | 23:28056245 | 97472 | 23:28056261 |
| 97473 | 23:28056279 | 97474 | 23:28058594 | 97475 | 23:28084930 | 97476 | 23:28084959 | 97480 | 23:28142109 |
| 97481 | 23:28142880 | 97482 | 23:28143776 | 97483 | 23:28147241 | 97485 | 23:28154077 | 97486 | 23:28154081 |
| 97487 | 23:28154567 | 97488 | 23:28158031 | 97489 | 23:28162469 | 97490 | 23:28169587 | 97492 | 23:28176948 |
| 97493 | 23:28177862 | 97494 | 23:28178986 | 97495 | 23:28180501 | 97496 | 23:28189617 | 97497 | 23:28214057 |
| 97499 | 23:28218286 | 97500 | 23:28226442 | 97502 | 23:28247153 | 97504 | 23:28252270 | 97505 | 23:28260136 |
| 97506 | 23:28261564 | 97507 | 23:28266105 | 97511 | 23:28289161 | 97512 | 23:28311837 | 97513 | 23:28314357 |
| 97514 | 23:28315369 | 97517 | 23:28344384 | 97518 | 23:28345650 | 97520 | 23:28370592 | 97522 | 23:28424137 |
| 97523 | 23:28428619 | 97525 | 23:28451722 | 97529 | 23:28527686 | 97530 | 23:28605105 | 97532 | 23:28614214 |
| 97533 | 23:28633036 | 97534 | 23:28644648 | 97535 | 23:28667350 | 97536 | 23:28759020 | 97537 | 23:28768059 |
| 97538 | 23:28770375 | 97539 | 23:28770402 | 97540 | 23:28770465 | 97541 | 23:28770499 | 97542 | 23:28770500 |
| 97543 | 23:28770527 | 97545 | 23:28779774 | 97547 | 23:28784189 | 97549 | 23:28791635 | 97551 | 23:28804972 |
| 97552 | 23:28806779 | 97553 | 23:28808892 | 97554 | 23:28811155 | 97555 | 23:28811333 | 97556 | 23:28817232 |
| 97557 | 23:28819161 | 97558 | 23:28819173 | 97560 | 23:28820599 | 97561 | 23:28821000 | 97563 | 23:28829534 |
| 97564 | 23:28836068 | 97565 | 23:28840263 | 97567 | 23:28855995 | 97568 | 23:28868586 | 97569 | 23:28870611 |
| 97570 | 23:28873879 | 97571 | 23:28878589 | 97572 | 23:28881735 | 97574 | 23:28920628 | 97575 | 23:28935375 |
| 97576 | 23:28938250 | 97580 | 23:28964584 | 97581 | 23:28967619 | 97582 | 23:28971358 | 97583 | 23:28980855 |
| 97584 | 23:29001677 | 97585 | 23:29002896 | 97586 | 23:29003973 | 97587 | 23:29004861 | 97589 | 23:29028277 |
| 97591 | 23:29088900 | 97593 | 23:29127784 | 97595 | 23:29155100 | 97598 | 23:29200199 | 97599 | 23:29231778 |
| 97604 | 23:29351343 | 97605 | 23:29351673 | 97606 | 23:29387809 | 97608 | 23:29405131 | 97610 | 23:29505622 |
| 97618 | 23:29739156 | 97619 | 23:29739161 | 97629 | 23:30015425 | 97633 | 23:30085624 | 97641 | 23:30343016 |
| 97643 | 23:30357104 | 97644 | 23:30377231 | 97645 | 23:30377501 | 97646 | 23:30391263 | 97647 | 23:30408989 |
| 97648 | 23:30411874 | 97649 | 23:30417585 | 97650 | 23:30422451 | 97651 | 23:30442710 | 97652 | 23:30443193 |
| 97653 | 23:30447941 | 97654 | 23:30464791 | 97656 | 23:30478896 | 97660 | 23:30538186 | 97662 | 23:30568716 |
| 97669 | 23:30789137 | 97670 | 23:30789578 | 97671 | 23:30789711 | 97685 | 23:31163020 | 97687 | 23:31193034 |
| 97688 | 23:31201200 | 97689 | 23:31207608 | 97690 | 23:31237063 | 97691 | 23:31239018 | 97696 | 23:31408344 |
| 97697 | 23:31474420 | 97698 | 23:31478356 | 97699 | 23:31478871 | 97706 | 23:31666638 | 97708 | 23:31712475 |
| 97709 | 23:31713030 | 97710 | 23:31713883 | 97712 | 23:31795823 | 97716 | 23:31839586 | 97717 | 23:31847799 |
| 97718 | 23:31860518 | 97719 | 23:31864204 | 97720 | 23:31883086 | 97721 | 23:31885522 | 97723 | 23:31892464 |
| 97724 | 23:31898107 | 97731 | 23:32103028 | 97735 | 23:32155949 | 97748 | 23:32348823 | 97754 | 23:32429180 |
| 97755 | 23:32429299 | 97756 | 23:32429437 | 97760 | 23:32498379 | 97763 | 23:32587067 | 97769 | 23:32714057 |
| 97776 | 23:32953339 | 97777 | 23:32953359 | 97778 | 23:32954768 | 97779 | 23:32963026 | 97781 | 23:32991047 |
| 97782 | 23:33010094 | 97783 | 23:33010248 | 97784 | 23:33017162 | 97793 | 23:33209372 | 97796 | 23:33232305 |
| 97809 | 23:33564304 | 97823 | 23:33923637 | 97829 | 23:34147241 | 97832 | 23:34255293 | 97846 | 23:34559019 |
| 97847 | 23:34568421 | 97872 | 23:35579146 | 97874 | 23:35643122 | 97882 | 23:35853906 | 97884 | 23:35933978 |
| 97897 | 23:36340613 | 97903 | 23:36491282 | 97904 | 23:36522078 | 97911 | 23:36715268 | 97922 | 23:37003285 |
| 97923 | 23:37008791 | 97926 | 23:37147881 | 97929 | 23:37226966 | 97931 | 23:37255744 | 97933 | 23:37290169 |
| 97934 | 23:37311602 | 97938 | 23:37438508 | 97939 | 23:37509546 | 97948 | 23:37644241 | 97952 | 23:37725066 |
| 97954 | 23:37743052 | 97964 | 23:38103066 | 97969 | 23:38331199 | 97970 | 23:38380507 | 97973 | 23:38417336 |
| 97995 | 23:38995562 | 98005 | 23:39248806 | 98007 | 23:39300011 | 98009 | 23:39319903 | 98029 | 23:39426076 |
| 98034 | 23:39454222 | 98046 | 23:39542549 | 98058 | 23:39582084 | 98065 | 23:39599289 | 98072 | 23:39623727 |
| 98074 | 23:39636483 | 98076 | 23:39640935 | 98083 | 23:39789176 | 98085 | 23:39815156 | 98090 | 23:39928196 |
| 98093 | 23:39972622 | 98097 | 23:40044291 | 98098 | 23:40064417 | 98106 | 23:40177352 | 98107 | 23:40190398 |
| 98109 | 23:40223350 | 98111 | 23:40255790 | 98112 | 23:40270530 | 98116 | 23:40376606 | 98119 | 23:40424669 |
| 98121 | 23:40430850 | 98123 | 23:40480107 | 98124 | 23:40500673 | 98125 | 23:40564407 | 98131 | 23:40662422 |
| 98133 | 23:40688993 | 98136 | 23:40758273 | 98139 | 23:40791971 | 98156 | 23:41037376 | 98161 | 23:41085635 |
| 98163 | 23:41110764 | 98177 | 23:41370057 | 98178 | 23:41371667 | 98179 | 23:41393226 | 98182 | 23:41488055 |
| 98198 | 23:41873620 | 98228 | 23:42474522 | 98251 | 23:43050343 | 98257 | 23:43142746 | 98266 | 23:43402438 |
| 98282 | 23:43699449 | 98288 | 23:43890903 | 98290 | 23:43922804 | 98325 | 23:44517863 | 98326 | 23:44517941 |
| 98332 | 23:44610396 | 98341 | 23:44856499 | 98343 | 23:44879028 | 98344 | 23:44896794 | 98345 | 23:44914975 |
| 98346 | 23:44944349 | 98354 | 23:45035699 | 98357 | 23:45057099 | 98358 | 23:45063024 | 98378 | 23:45357440 |
| 98380 | 23:45397415 | 98381 | 23:45398681 | 98383 | 23:45430124 | 98384 | 23:45432927 | 98390 | 23:45560731 |
| 98404 | 23:46021030 | 98405 | 23:46022790 | 98406 | 23:46022913 | 98407 | 23:46023921 | 98408 | 23:46024140 |
| 98419 | 23:46342707 | 98424 | 23:46476087 | 98434 | 23:46728089 | 98439 | 23:46821712 | 98440 | 23:46867745 |
| 98445 | 23:46932972 | 98447 | 23:46982531 | 98448 | 23:46982538 | 98449 | 23:46982544 | 98450 | 23:46982553 |
| 98451 | 23:46982569 | 98452 | 23:46982672 | 98453 | 23:46982688 | 98469 | 23:47346112 | 98470 | 23:47346172 |
| 98472 | 23:47377820 | 98476 | 23:47501209 | 98479 | 23:47512112 | 98480 | 23:47535388 | 98493 | 23:47816262 |
| 98494 | 23:47825640 | 98495 | 23:47825678 | 98496 | 23:47826029 | 98497 | 23:47826560 | 98498 | 23:47827373 |
| 98500 | 23:47839983 | 98501 | 23:47840868 | 98503 | 23:47845586 | 98504 | 23:47847036 | 98523 | 23:48330566 |
| 98529 | 23:48410101 | 98533 | 23:48581773 | 98538 | 23:48634859 | 98545 | 23:48734960 | 98547 | 23:48752878 |
| 98555 | 23:48901109 | 98567 | 23:49079717 | 98569 | 23:49107918 | 98574 | 23:49198514 | 98576 | 23:49210048 |
| 98584 | 23:49312924 | 98585 | 23:49312984 | 98586 | 23:49312985 | 98587 | 23:49312986 | 98592 | 23:49424994 |
| 98593 | 23:49432035 | 98594 | 23:49442339 | 98602 | 23:49590910 | 98622 | 23:49941067 | 98624 | 23:49942903 |
| 98625 | 23:49942904 | 98632 | 23:50043273 | 98635 | 23:50078664 | 98636 | 23:50114133 | 98638 | 23:50169252 |
| 98642 | 23:50258011 | 98649 | 23:50375296 | 98664 | 23:50638453 | 98671 | 23:50875493 | 98678 | 23:50932969 |
| 98680 | 23:50964263 | 98686 | 23:51018373 | 98689 | 23:51079827 | 98691 | 23:51116781 | 98701 | 23:51298091 |
| 98711 | 23:51501609 | 98718 | 23:51674082 | 98722 | 23:51744554 | 98728 | 23:51847760 | 98737 | 23:52008498 |
| 98741 | 23:52053452 | 98743 | 23:52076716 | 98781 | 24:469885 | 98798 | 24:880857 | 98799 | 24:888643 |
| 98802 | 24:930959 | 98805 | 24:980184 | 98808 | 24:1046300 | 98818 | 24:1170762 | 98819 | 24:1171661 |
| 98834 | 24:1570237 | 98851 | 24:1855510 | 98862 | 24:2006568 | 98871 | 24:2265624 | 98872 | 24:2276656 |
| 98900 | 24:2945776 | 98902 | 24:2978064 | 98918 | 24:3289918 | 98930 | 24:3538543 | 98933 | 24:3696514 |
| 98940 | 24:3908092 | 98942 | 24:3966055 | 98943 | 24:3966359 | 98947 | 24:4007654 | 98948 | 24:4007678 |
| 98949 | 24:4012661 | 98950 | 24:4013403 | 98951 | 24:4021260 | 98956 | 24:4164775 | 98957 | 24:4164916 |
| 98958 | 24:4164934 | 98959 | 24:4165918 | 98960 | 24:4168088 | 98965 | 24:4188159 | 98970 | 24:4291963 |
| 98974 | 24:4399899 | 98980 | 24:4581542 | 98982 | 24:4612976 | 98989 | 24:4963101 | 98995 | 24:5087606 |
| 99004 | 24:5305296 | 99005 | 24:5333407 | 99006 | 24:5337650 | 99018 | 24:5642653 | 99026 | 24:5818978 |
| 99038 | 24:6264925 | 99076 | 24:7162546 | 99078 | 24:7286305 | 99086 | 24:7467215 | 99111 | 24:8069004 |
| 99112 | 24:8108977 | 99113 | 24:8109036 | 99116 | 24:8121802 | 99118 | 24:8175362 | 99119 | 24:8182146 |
| 99146 | 24:9005035 | 99151 | 24:9085950 | 99190 | 24:9913417 | 99192 | 24:9977557 | 99276 | 24:10203460 |
| 99283 | 24:10336705 | 99286 | 24:10405660 | 99292 | 24:10590068 | 99293 | 24:10590091 | 99311 | 24:11064065 |
| 99321 | 24:11243732 | 99322 | 24:11285722 | 99329 | 24:11451899 | 99339 | 24:11738379 | 99359 | 24:12247620 |
| 99364 | 24:12466661 | 99365 | 24:12471212 | 99371 | 24:12650426 | 99372 | 24:12652362 | 99373 | 24:12660326 |
| 99396 | 24:13228420 | 99405 | 24:13465765 | 99418 | 24:13789354 | 99419 | 24:13790831 | 99420 | 24:13791730 |
| 99427 | 24:14119618 | 99459 | 24:14988931 | 99484 | 24:15813747 | 99490 | 24:16186692 | 99501 | 24:16499172 |
| 99503 | 24:16506987 | 99509 | 24:16666243 | 99528 | 24:17162739 | 99530 | 24:17208682 | 99543 | 24:17625014 |
| 99565 | 24:18353069 | 99574 | 24:18678355 | 99598 | 24:19285724 | 99603 | 24:19437043 | 99607 | 24:19606961 |
| 99610 | 24:19713861 | 99615 | 24:19770957 | 99628 | 24:20349623 | 99629 | 24:20358408 | 99632 | 24:20393549 |
| 99638 | 24:20499578 | 99639 | 24:20510429 | 99640 | 24:20510450 | 99641 | 24:20510487 | 99642 | 24:20510488 |
| 99643 | 24:20510505 | 99644 | 24:20510579 | 99645 | 24:20510583 | 99646 | 24:20510587 | 99647 | 24:20510597 |
| 99648 | 24:20510600 | 99649 | 24:20513392 | 99650 | 24:20513424 | 99651 | 24:20513517 | 99652 | 24:20521127 |
| 99653 | 24:20532438 | 99654 | 24:20532441 | 99655 | 24:20532446 | 99656 | 24:20532502 | 99657 | 24:20572686 |
| 99669 | 24:20847626 | 99671 | 24:20874971 | 99672 | 24:20920443 | 99673 | 24:20920491 | 99674 | 24:20920727 |
| 99675 | 24:20922777 | 99676 | 24:20926914 | 99677 | 24:20927000 | 99678 | 24:20927024 | 99679 | 24:20927059 |
| 99680 | 24:20929398 | 99681 | 24:20929449 | 99682 | 24:20929473 | 99693 | 24:21272688 | 99695 | 24:21306399 |
| 99696 | 24:21375203 | 99697 | 24:21376904 | 99698 | 24:21376940 | 99699 | 24:21376967 | 99700 | 24:21376972 |
| 99701 | 24:21378281 | 99702 | 24:21378321 | 99703 | 24:21378352 | 99704 | 24:21378382 | 99705 | 24:21378383 |
| 99706 | 24:21378384 | 99708 | 24:21378440 | 99709 | 24:21378445 | 99711 | 24:21395177 | 99712 | 24:21396591 |
| 99716 | 24:21432058 | 99717 | 24:21452284 | 99718 | 24:21456850 | 99719 | 24:21471419 | 99723 | 24:21488223 |
| 99725 | 24:21504166 | 99729 | 24:21560026 | 99730 | 24:21560060 | 99732 | 24:21564234 | 99733 | 24:21587057 |
| 99734 | 24:21590358 | 99735 | 24:21595564 | 99736 | 24:21598260 | 99737 | 24:21605659 | 99738 | 24:21620362 |
| 99739 | 24:21667119 | 99740 | 24:21721258 | 99742 | 24:21765426 | 99754 | 24:21940194 | 99756 | 24:21971534 |
| 99762 | 24:22139528 | 99769 | 24:22384770 | 99777 | 24:22507325 | 99779 | 24:22552252 | 99786 | 24:22901733 |
| 99787 | 24:22901743 | 99788 | 24:22901792 | 99789 | 24:22901810 | 99790 | 24:22901819 | 99795 | 24:23015622 |
| 99797 | 24:23048740 | 99798 | 24:23048839 | 99800 | 24:23138496 | 99808 | 24:23304844 | 99809 | 24:23357754 |
| 99812 | 24:23476124 | 99840 | 24:24470786 | 99848 | 24:24601724 | 99850 | 24:24651791 | 99856 | 24:24861946 |
| 99858 | 24:24942299 | 99859 | 24:24968711 | 99864 | 24:25055928 | 99867 | 24:25166673 | 99870 | 24:25259209 |
| 99871 | 24:25291130 | 99875 | 24:25393251 | 99880 | 24:25549639 | 99881 | 24:25550670 | 99882 | 24:25554087 |
| 99883 | 24:25555252 | 99884 | 24:25586246 | 99887 | 24:25655955 | 99891 | 24:25728080 | 99892 | 24:25730700 |
| 99893 | 24:25732798 | 99895 | 24:25765374 | 99901 | 24:25840462 | 99928 | 24:26414878 | 99929 | 24:26414942 |
| 99939 | 24:26752400 | 99960 | 24:27335211 | 99976 | 24:27659643 | 99977 | 24:27671338 | 99978 | 24:27707920 |
| 99987 | 24:27976475 | 99992 | 24:28073538 | 100015 | 24:28717465 | 100025 | 24:28990565 | 100026 | 24:28990603 |
| 100027 | 24:28990620 | 100030 | 24:29071264 | 100034 | 24:29205694 | 100038 | 24:29294106 | 100040 | 24:29309403 |
| 100050 | 24:29593728 | 100055 | 24:29785127 | 100065 | 24:30022971 | 100066 | 24:30028664 | 100068 | 24:30028736 |
| 100074 | 24:30132653 | 100087 | 24:30418113 | 100101 | 24:30736100 | 100102 | 24:30756120 | 100103 | 24:30756121 |
| 100115 | 24:31302891 | 100118 | 24:31387621 | 100129 | 24:31602471 | 100145 | 24:31910298 | 100158 | 24:32159647 |
| 100159 | 24:32159730 | 100165 | 24:32335210 | 100172 | 24:32561115 | 100178 | 24:32732872 | 100179 | 24:32732892 |
| 100180 | 24:32732952 | 100181 | 24:32735243 | 100183 | 24:32769877 | 100186 | 24:32810095 | 100188 | 24:32824750 |
| 100189 | 24:32826968 | 100190 | 24:32827828 | 100191 | 24:32830047 | 100192 | 24:32830055 | 100193 | 24:32840723 |
| 100194 | 24:32857855 | 100197 | 24:32868393 | 100198 | 24:32875942 | 100200 | 24:32894247 | 100209 | 24:33166170 |
| 100211 | 24:33227163 | 100212 | 24:33227191 | 100231 | 24:33780521 | 100254 | 24:34326602 | 100256 | 24:34364918 |
| 100264 | 24:34614458 | 100268 | 24:34760712 | 100269 | 24:34762120 | 100271 | 24:34813345 | 100284 | 24:35185894 |
| 100286 | 24:35185970 | 100291 | 24:35309422 | 100292 | 24:35335089 | 100299 | 24:35472794 | 100321 | 24:35837548 |
| 100346 | 24:36589347 | 100347 | 24:36589477 | 100353 | 24:36835198 | 100357 | 24:36988061 | 100360 | 24:37134995 |
| 100365 | 24:37239650 | 100367 | 24:37280274 | 100368 | 24:37304503 | 100369 | 24:37306947 | 100375 | 24:37417740 |
| 100382 | 24:37592997 | 100386 | 24:37706524 | 100394 | 24:37931336 | 100406 | 24:38475906 | 100409 | 24:38609603 |
| 100413 | 24:38809089 | 100420 | 24:38949417 | 100430 | 24:39200562 | 100432 | 24:39308113 | 100438 | 24:39456209 |
| 100439 | 24:39464085 | 100442 | 24:39492963 | 100448 | 24:39687343 | 100450 | 24:39707191 | 100458 | 24:39897863 |
| 100459 | 24:39902640 | 100460 | 24:39906046 | 100461 | 24:39906190 | 100462 | 24:39913847 | 100476 | 24:40288339 |
| 100492 | 24:40342848 | 100495 | 24:40352389 | 100497 | 24:40395247 | 100499 | 24:40408675 | 100504 | 24:40466974 |
| 100508 | 24:40545027 | 100509 | 24:40573682 | 100510 | 24:40583210 | 100511 | 24:40583246 | 100512 | 24:40587358 |
| 100519 | 24:40746951 | 100520 | 24:40756834 | 100522 | 24:40764626 | 100525 | 24:40781770 | 100526 | 24:40798538 |
| 100527 | 24:40803087 | 100534 | 24:40974776 | 100540 | 24:41093545 | 100545 | 24:41180993 | 100547 | 24:41204807 |
| 100566 | 24:41805610 | 100573 | 24:41935257 | 100574 | 24:41944725 | 100579 | 24:42049855 | 100581 | 24:42141779 |
| 100585 | 24:42231198 | 100586 | 24:42251623 | 100588 | 24:42272285 | 100589 | 24:42276093 | 100592 | 24:42324465 |
| 100599 | 24:42659053 | 100600 | 24:42659123 | 100601 | 24:42659180 | 100602 | 24:42659202 | 100604 | 24:42709614 |
| 100605 | 24:42721124 | 100606 | 24:42724162 | 100607 | 24:42725838 | 100608 | 24:42726075 | 100609 | 24:42727665 |
| 100611 | 24:42768635 | 100612 | 24:42779119 | 100614 | 24:42816514 | 100615 | 24:42881112 | 100633 | 24:43435997 |
| 100636 | 24:43500003 | 100647 | 24:44069431 | 100656 | 24:44298375 | 100661 | 24:44375642 | 100662 | 24:44396418 |
| 100668 | 24:44471971 | 100677 | 24:44658910 | 100679 | 24:44735016 | 100681 | 24:44775120 | 100682 | 24:44792435 |
| 100697 | 24:45080627 | 100704 | 24:45199003 | 100706 | 24:45243583 | 100713 | 24:45357351 | 100716 | 24:45461858 |
| 100718 | 24:45489006 | 100719 | 24:45499596 | 100720 | 24:45501942 | 100722 | 24:45537440 | 100728 | 24:45683209 |
| 100730 | 24:45738887 | 100732 | 24:45745474 | 100734 | 24:45809276 | 100735 | 24:45824753 | 100749 | 24:45922636 |
| 100750 | 24:45923194 | 100758 | 24:46005539 | 100770 | 24:46107365 | 100771 | 24:46128800 | 100772 | 24:46131144 |
| 100778 | 24:46200299 | 100781 | 24:46293419 | 100786 | 24:46339304 | 100787 | 24:46339449 | 100788 | 24:46343493 |
| 100789 | 24:46349257 | 100799 | 24:46658398 | 100801 | 24:46735235 | 100802 | 24:46748616 | 100806 | 24:46860941 |
| 100807 | 24:46863126 | 100809 | 24:46872067 | 100810 | 24:46872779 | 100811 | 24:46872783 | 100812 | 24:46872835 |
| 100813 | 24:46874235 | 100823 | 24:47186952 | 100835 | 24:47493053 | 100836 | 24:47504673 | 100839 | 24:47575681 |
| 100846 | 24:47792768 | 100849 | 24:47861526 | 100854 | 24:47942540 | 100862 | 24:48132259 | 100866 | 24:48283076 |
| 100870 | 24:48398019 | 100871 | 24:48400794 | 100885 | 24:48949533 | 100894 | 24:49153895 | 100897 | 24:49213874 |
| 100899 | 24:49220538 | 100901 | 24:49271697 | 100904 | 24:49386058 | 100905 | 24:49461650 | 100917 | 24:49808792 |
| 100923 | 24:49949844 | 100924 | 24:49951357 | 100925 | 24:49954724 | 100935 | 24:50278508 | 100936 | 24:50323489 |
| 100937 | 24:50330708 | 100945 | 24:50485176 | 100950 | 24:50664362 | 100958 | 24:50886055 | 100964 | 24:51077466 |
| 100965 | 24:51079480 | 100983 | 24:51488836 | 101010 | 24:51956661 | 101016 | 24:52119070 | 101022 | 24:52251430 |
| 101023 | 24:52257462 | 101024 | 24:52257497 | 101047 | 24:52725727 | 101053 | 24:53039441 | 101054 | 24:53060437 |
| 101069 | 24:53471017 | 101070 | 24:53476039 | 101072 | 24:53480782 | 101074 | 24:53485369 | 101075 | 24:53489339 |
| 101081 | 24:53695135 | 101098 | 24:54141567 | 101102 | 24:54171685 | 101108 | 24:54227463 | 101115 | 24:54361200 |
| 101117 | 24:54366075 | 101123 | 24:54596604 | 101124 | 24:54605333 | 101130 | 24:54678133 | 101138 | 24:54772296 |
| 101140 | 24:54837850 | 101141 | 24:54844868 | 101142 | 24:54868156 | 101162 | 24:55295472 | 101163 | 24:55304369 |
| 101178 | 24:55700714 | 101180 | 24:55732464 | 101181 | 24:55736959 | 101182 | 24:55739826 | 101188 | 24:55923426 |
| 101191 | 24:56024340 | 101192 | 24:56024373 | 101193 | 24:56024406 | 101197 | 24:56098690 | 101198 | 24:56102284 |
| 101199 | 24:56102647 | 101200 | 24:56102653 | 101201 | 24:56102851 | 101227 | 24:56688810 | 101228 | 24:56746296 |
| 101229 | 24:56746319 | 101232 | 24:56797648 | 101233 | 24:56813204 | 101255 | 24:57240950 | 101258 | 24:57371376 |
| 101259 | 24:57399764 | 101261 | 24:57412925 | 101269 | 24:57552725 | 101270 | 24:57558999 | 101275 | 24:57626126 |
| 101281 | 24:57747906 | 101283 | 24:57797863 | 101295 | 24:58058760 | 101299 | 24:58124009 | 101316 | 24:58425089 |
| 101320 | 24:58468125 | 101326 | 24:58577110 | 101328 | 24:58601846 | 101341 | 24:58910690 | 101352 | 24:59164671 |
| 101353 | 24:59196985 | 101361 | 24:59421526 | 101363 | 24:59468734 | 101381 | 24:59829538 | 101421 | 24:60586093 |
| 101424 | 24:60623865 | 101425 | 24:60632706 | 101442 | 24:60989011 | 101443 | 24:61029712 | 101445 | 24:61052179 |
| 101449 | 24:61119884 | 101454 | 24:61260912 | 101463 | 24:61463290 | 101464 | 24:61466635 | 101466 | 24:61477326 |
| 101468 | 24:61528992 | 101471 | 24:61571637 | 101479 | 24:61692816 | 101483 | 24:61746382 | 101506 | 25:121555 |
| 101507 | 25:127561 | 101517 | 25:343439 | 101519 | 25:373190 | 101520 | 25:376453 | 101521 | 25:424297 |
| 101524 | 25:567175 | 101525 | 25:572006 | 101528 | 25:670011 | 101529 | 25:670169 | 101541 | 25:950550 |
| 101544 | 25:1045088 | 101546 | 25:1087084 | 101549 | 25:1134920 | 101550 | 25:1139788 | 101552 | 25:1177714 |
| 101556 | 25:1296800 | 101557 | 25:1324226 | 101560 | 25:1352107 | 101562 | 25:1353932 | 101563 | 25:1354851 |
| 101564 | 25:1364554 | 101565 | 25:1364583 | 101566 | 25:1365638 | 101570 | 25:1405987 | 101573 | 25:1476171 |
| 101574 | 25:1478625 | 101581 | 25:1649137 | 101588 | 25:1775261 | 101589 | 25:1777637 | 101590 | 25:1777709 |
| 101591 | 25:1777710 | 101592 | 25:1777721 | 101593 | 25:1783080 | 101595 | 25:1790972 | 101596 | 25:1791028 |
| 101597 | 25:1808653 | 101599 | 25:1818249 | 101600 | 25:1835147 | 101602 | 25:1862652 | 101603 | 25:1883004 |
| 101608 | 25:1966690 | 101611 | 25:2003071 | 101620 | 25:2200434 | 101626 | 25:2247195 | 101659 | 25:2376504 |
| 101661 | 25:2422717 | 101665 | 25:2477784 | 101666 | 25:2479395 | 101667 | 25:2479688 | 101668 | 25:2479754 |
| 101670 | 25:2537288 | 101671 | 25:2538507 | 101672 | 25:2540535 | 101673 | 25:2547137 | 101674 | 25:2548630 |
| 101675 | 25:2552738 | 101676 | 25:2553655 | 101677 | 25:2555768 | 101678 | 25:2560887 | 101679 | 25:2561123 |
| 101680 | 25:2563116 | 101681 | 25:2564127 | 101683 | 25:2565592 | 101684 | 25:2568326 | 101685 | 25:2573673 |
| 101686 | 25:2575681 | 101687 | 25:2578879 | 101688 | 25:2580437 | 101689 | 25:2581459 | 101690 | 25:2582592 |
| 101691 | 25:2585032 | 101692 | 25:2586441 | 101700 | 25:2681110 | 101701 | 25:2681129 | 101703 | 25:2687754 |
| 101706 | 25:2695684 | 101707 | 25:2695701 | 101708 | 25:2695795 | 101709 | 25:2695845 | 101710 | 25:2695937 |
| 101711 | 25:2696055 | 101712 | 25:2696181 | 101713 | 25:2696410 | 101714 | 25:2698504 | 101715 | 25:2699035 |
| 101716 | 25:2699105 | 101717 | 25:2739170 | 101718 | 25:2740282 | 101719 | 25:2740291 | 101721 | 25:2743006 |
| 101722 | 25:2743075 | 101723 | 25:2743261 | 101724 | 25:2743406 | 101725 | 25:2749450 | 101727 | 25:2762252 |
| 101728 | 25:2762309 | 101729 | 25:2775821 | 101732 | 25:2819065 | 101733 | 25:2819396 | 101734 | 25:2819471 |
| 101735 | 25:2819522 | 101736 | 25:2819528 | 101737 | 25:2819546 | 101738 | 25:2819603 | 101750 | 25:3210540 |
| 101758 | 25:3310916 | 101760 | 25:3363490 | 101763 | 25:3426286 | 101767 | 25:3477152 | 101770 | 25:3524368 |
| 101771 | 25:3529015 | 101775 | 25:3534470 | 101776 | 25:3534510 | 101777 | 25:3554022 | 101780 | 25:3581070 |
| 101781 | 25:3588116 | 101782 | 25:3595733 | 101783 | 25:3604536 | 101784 | 25:3606474 | 101785 | 25:3625688 |
| 101786 | 25:3630953 | 101787 | 25:3640402 | 101788 | 25:3650316 | 101789 | 25:3656324 | 101792 | 25:3701510 |
| 101795 | 25:3830536 | 101797 | 25:3851824 | 101800 | 25:3903874 | 101804 | 25:4088166 | 101816 | 25:4278985 |
| 101818 | 25:4319018 | 101853 | 25:5024647 | 101861 | 25:5186113 | 101867 | 25:5326044 | 101917 | 25:5967822 |
| 101921 | 25:6012533 | 101932 | 25:6175707 | 101934 | 25:6231384 | 101959 | 25:6788634 | 101960 | 25:6838881 |
| 101962 | 25:6862509 | 101963 | 25:6862547 | 101980 | 25:7313080 | 101988 | 25:7484309 | 101989 | 25:7517426 |
| 101995 | 25:7734903 | 101998 | 25:7840771 | 101999 | 25:7840811 | 102004 | 25:7884771 | 102008 | 25:7905114 |
| 102010 | 25:7941201 | 102014 | 25:8064816 | 102025 | 25:8388133 | 102029 | 25:8511991 | 102046 | 25:8788906 |
| 102047 | 25:8844118 | 102056 | 25:9197230 | 102060 | 25:9300591 | 102064 | 25:9365404 | 102067 | 25:9378741 |
| 102068 | 25:9385962 | 102070 | 25:9502049 | 102075 | 25:9643363 | 102078 | 25:9687538 | 102082 | 25:9776752 |
| 102084 | 25:9843435 | 102091 | 25:9993793 | 102092 | 25:10007062 | 102102 | 25:10139839 | 102106 | 25:10198288 |
| 102108 | 25:10227883 | 102109 | 25:10272497 | 102112 | 25:10365727 | 102118 | 25:10541563 | 102119 | 25:10541576 |
| 102120 | 25:10547353 | 102131 | 25:10832103 | 102132 | 25:10836311 | 102136 | 25:10948241 | 102137 | 25:10951982 |
| 102142 | 25:11012348 | 102147 | 25:11110219 | 102148 | 25:11111271 | 102150 | 25:11138838 | 102162 | 25:11274257 |
| 102168 | 25:11436742 | 102169 | 25:11437820 | 102172 | 25:11447709 | 102173 | 25:11455980 | 102177 | 25:11549424 |
| 102178 | 25:11549497 | 102179 | 25:11570419 | 102207 | 25:12214562 | 102215 | 25:12401343 | 102235 | 25:12943128 |
| 102244 | 25:13251854 | 102248 | 25:13339029 | 102249 | 25:13345472 | 102252 | 25:13388375 | 102262 | 25:13601294 |
| 102273 | 25:14013151 | 102280 | 25:14073120 | 102286 | 25:14201547 | 102292 | 25:14377407 | 102293 | 25:14409676 |
| 102294 | 25:14448614 | 102299 | 25:14511967 | 102301 | 25:14519922 | 102306 | 25:14583409 | 102310 | 25:14703237 |
| 102316 | 25:14862354 | 102328 | 25:15092643 | 102334 | 25:15276545 | 102335 | 25:15276571 | 102338 | 25:15370203 |
| 102351 | 25:15564906 | 102353 | 25:15633665 | 102354 | 25:15633709 | 102355 | 25:15633785 | 102365 | 25:15912161 |
| 102367 | 25:15942929 | 102369 | 25:15943009 | 102371 | 25:15996288 | 102377 | 25:16093653 | 102383 | 25:16219145 |
| 102387 | 25:16288361 | 102388 | 25:16288367 | 102397 | 25:16429229 | 102400 | 25:16494465 | 102423 | 25:17127180 |
| 102437 | 25:17439767 | 102438 | 25:17446840 | 102440 | 25:17474953 | 102441 | 25:17475010 | 102442 | 25:17483539 |
| 102443 | 25:17488467 | 102457 | 25:17838540 | 102463 | 25:18019077 | 102465 | 25:18091054 | 102470 | 25:18283899 |
| 102471 | 25:18285948 | 102482 | 25:18730651 | 102486 | 25:18747611 | 102487 | 25:18760366 | 102489 | 25:18798985 |
| 102499 | 25:18976796 | 102500 | 25:18988158 | 102532 | 25:19881913 | 102536 | 25:19929634 | 102540 | 25:20012375 |
| 102542 | 25:20043037 | 102543 | 25:20085662 | 102550 | 25:20338297 | 102575 | 25:21049790 | 102578 | 25:21139359 |
| 102584 | 25:21196203 | 102587 | 25:21260648 | 102589 | 25:21317697 | 102591 | 25:21318468 | 102592 | 25:21327438 |
| 102593 | 25:21329507 | 102597 | 25:21405459 | 102601 | 25:21506832 | 102603 | 25:21524798 | 102604 | 25:21532563 |
| 102629 | 25:22103985 | 102642 | 25:22376703 | 102643 | 25:22394285 | 102644 | 25:22420195 | 102655 | 25:22670949 |
| 102680 | 25:23399437 | 102694 | 25:23750537 | 102712 | 25:24033949 | 102716 | 25:24132273 | 102722 | 25:24242643 |
| 102726 | 25:24361949 | 102743 | 25:24619856 | 102748 | 25:24768804 | 102750 | 25:24829402 | 102751 | 25:24857183 |
| 102755 | 25:24902475 | 102757 | 25:24915980 | 102758 | 25:24918063 | 102759 | 25:24918979 | 102760 | 25:24918980 |
| 102761 | 25:24920501 | 102771 | 25:25159107 | 102779 | 25:25226497 | 102780 | 25:25231198 | 102781 | 25:25252790 |
| 102784 | 25:25319545 | 102785 | 25:25319598 | 102786 | 25:25327412 | 102787 | 25:25327437 | 102788 | 25:25355320 |
| 102789 | 25:25355323 | 102790 | 25:25370729 | 102800 | 25:25518754 | 102802 | 25:25644509 | 102804 | 25:25729230 |
| 102806 | 25:25764911 | 102818 | 25:26048984 | 102834 | 25:26491613 | 102835 | 25:26491635 | 102836 | 25:26491678 |
| 102837 | 25:26491720 | 102838 | 25:26491762 | 102840 | 25:26492004 | 102841 | 25:26492080 | 102842 | 25:26492086 |
| 102843 | 25:26492143 | 102844 | 25:26493056 | 102845 | 25:26493215 | 102846 | 25:26493378 | 102858 | 25:26873278 |
| 102859 | 25:26879815 | 102862 | 25:26957845 | 102866 | 25:27074581 | 102867 | 25:27078879 | 102868 | 25:27079915 |
| 102869 | 25:27081813 | 102881 | 25:27471780 | 102883 | 25:27505633 | 102884 | 25:27529561 | 102886 | 25:27546363 |
| 102888 | 25:27577974 | 102889 | 25:27610631 | 102890 | 25:27614146 | 102891 | 25:27621694 | 102892 | 25:27623762 |
| 102893 | 25:27634139 | 102894 | 25:27647855 | 102895 | 25:27651029 | 102896 | 25:27654242 | 102897 | 25:27671337 |
| 102898 | 25:27672683 | 102899 | 25:27677306 | 102902 | 25:27724958 | 102903 | 25:27728373 | 102905 | 25:27731091 |
| 102906 | 25:27734591 | 102907 | 25:27737084 | 102908 | 25:27743420 | 102909 | 25:27747175 | 102910 | 25:27747494 |
| 102911 | 25:27747510 | 102912 | 25:27748521 | 102913 | 25:27749865 | 102914 | 25:27749919 | 102915 | 25:27749952 |
| 102916 | 25:27749994 | 102917 | 25:27750315 | 102918 | 25:27750769 | 102919 | 25:27751432 | 102921 | 25:27790340 |
| 102931 | 25:28024158 | 102933 | 25:28057161 | 102936 | 25:28188815 | 102941 | 25:28274850 | 102944 | 25:28376458 |
| 102950 | 25:28539043 | 102958 | 25:28760002 | 102970 | 25:29158123 | 102985 | 25:29422226 | 102989 | 25:29507318 |
| 102992 | 25:29574598 | 102997 | 25:29653196 | 103000 | 25:29711516 | 103018 | 25:29924846 | 103021 | 25:29960827 |
| 103023 | 25:29983402 | 103027 | 25:30108430 | 103030 | 25:30188316 | 103061 | 25:31059775 | 103082 | 25:31397966 |
| 103083 | 25:31397972 | 103120 | 25:32101198 | 103121 | 25:32115103 | 103122 | 25:32122180 | 103130 | 25:32225777 |
| 103148 | 25:32620702 | 103149 | 25:32636956 | 103151 | 25:32651385 | 103155 | 25:32784599 | 103156 | 25:32785820 |
| 103158 | 25:32838124 | 103166 | 25:33064837 | 103167 | 25:33068854 | 103177 | 25:33278840 | 103179 | 25:33325832 |
| 103188 | 25:33472178 | 103193 | 25:33614940 | 103194 | 25:33626966 | 103205 | 25:33862553 | 103208 | 25:33918451 |
| 103209 | 25:33918614 | 103211 | 25:33933997 | 103213 | 25:33986857 | 103221 | 25:34165756 | 103227 | 25:34324511 |
| 103231 | 25:34373185 | 103232 | 25:34380738 | 103233 | 25:34390163 | 103235 | 25:34415206 | 103236 | 25:34416256 |
| 103237 | 25:34423242 | 103238 | 25:34430800 | 103241 | 25:34512804 | 103242 | 25:34523864 | 103244 | 25:34541030 |
| 103247 | 25:34607593 | 103248 | 25:34616589 | 103250 | 25:34627570 | 103256 | 25:34729070 | 103269 | 25:34979341 |
| 103270 | 25:34987197 | 103272 | 25:35035356 | 103274 | 25:35047480 | 103278 | 25:35162155 | 103285 | 25:35220858 |
| 103286 | 25:35223524 | 103287 | 25:35224634 | 103294 | 25:35416991 | 103296 | 25:35510566 | 103298 | 25:35538895 |
| 103299 | 25:35539311 | 103302 | 25:35574079 | 103303 | 25:35596429 | 103307 | 25:35632501 | 103310 | 25:35695366 |
| 103311 | 25:35695667 | 103312 | 25:35695717 | 103313 | 25:35696225 | 103314 | 25:35696282 | 103315 | 25:35697157 |
| 103317 | 25:35723853 | 103318 | 25:35726457 | 103319 | 25:35737565 | 103320 | 25:35767708 | 103322 | 25:35774507 |
| 103323 | 25:35775008 | 103324 | 25:35784841 | 103329 | 25:35868596 | 103331 | 25:35874518 | 103332 | 25:35930268 |
| 103335 | 25:35960525 | 103344 | 25:36192757 | 103347 | 25:36214845 | 103348 | 25:36214858 | 103349 | 25:36215138 |
| 103350 | 25:36215175 | 103351 | 25:36215182 | 103352 | 25:36215303 | 103353 | 25:36215758 | 103354 | 25:36215759 |
| 103355 | 25:36216611 | 103356 | 25:36216901 | 103357 | 25:36216911 | 103358 | 25:36217079 | 103359 | 25:36217150 |
| 103360 | 25:36217198 | 103361 | 25:36217712 | 103362 | 25:36217751 | 103364 | 25:36225793 | 103365 | 25:36225817 |
| 103366 | 25:36246266 | 103367 | 25:36249624 | 103371 | 25:36344903 | 103372 | 25:36350043 | 103373 | 25:36360014 |
| 103379 | 25:36450152 | 103380 | 25:36450202 | 103383 | 25:36512847 | 103384 | 25:36531061 | 103385 | 25:36535022 |
| 103386 | 25:36538745 | 103388 | 25:36584800 | 103393 | 25:36811728 | 103398 | 25:36971262 | 103399 | 25:36978649 |
| 103400 | 25:36985203 | 103403 | 25:37031840 | 103404 | 25:37034930 | 103405 | 25:37043595 | 103406 | 25:37046117 |
| 103407 | 25:37060161 | 103408 | 25:37061145 | 103413 | 25:37136607 | 103414 | 25:37136612 | 103415 | 25:37138640 |
| 103416 | 25:37138651 | 103417 | 25:37138668 | 103419 | 25:37149974 | 103421 | 25:37184274 | 103424 | 25:37205234 |
| 103426 | 25:37227790 | 103427 | 25:37241594 | 103428 | 25:37242352 | 103429 | 25:37242939 | 103430 | 25:37242955 |
| 103431 | 25:37242995 | 103432 | 25:37243078 | 103434 | 25:37252529 | 103437 | 25:37356346 | 103442 | 25:37389979 |
| 103466 | 25:37729407 | 103467 | 25:37784956 | 103476 | 25:37964578 | 103481 | 25:38114076 | 103482 | 25:38114479 |
| 103483 | 25:38117206 | 103484 | 25:38118273 | 103485 | 25:38126701 | 103487 | 25:38166852 | 103488 | 25:38190732 |
| 103490 | 25:38227705 | 103491 | 25:38233097 | 103496 | 25:38300677 | 103497 | 25:38300772 | 103498 | 25:38300781 |
| 103501 | 25:38363440 | 103502 | 25:38394640 | 103503 | 25:38394741 | 103507 | 25:38489542 | 103520 | 25:38856435 |
| 103523 | 25:38930854 | 103530 | 25:39028696 | 103531 | 25:39045580 | 103541 | 25:39300696 | 103542 | 25:39317059 |
| 103543 | 25:39342146 | 103544 | 25:39345282 | 103548 | 25:39449897 | 103556 | 25:39552784 | 103573 | 25:39918879 |
| 103592 | 25:40240409 | 103593 | 25:40240500 | 103601 | 25:40328748 | 103605 | 25:40396102 | 103608 | 25:40553090 |
| 103613 | 25:40614314 | 103621 | 25:40720968 | 103624 | 25:40758343 | 103633 | 25:40876573 | 103635 | 25:40901328 |
| 103636 | 25:40903884 | 103639 | 25:40948048 | 103640 | 25:40954468 | 103641 | 25:40956329 | 103642 | 25:40960034 |
| 103643 | 25:40960925 | 103644 | 25:40979131 | 103645 | 25:40982964 | 103646 | 25:40991964 | 103647 | 25:40998163 |
| 103649 | 25:41007519 | 103650 | 25:41009480 | 103651 | 25:41012986 | 103652 | 25:41014477 | 103653 | 25:41015047 |
| 103654 | 25:41022922 | 103655 | 25:41028182 | 103656 | 25:41031444 | 103657 | 25:41033389 | 103658 | 25:41034653 |
| 103659 | 25:41039109 | 103661 | 25:41046191 | 103662 | 25:41066639 | 103663 | 25:41070067 | 103664 | 25:41074859 |
| 103674 | 25:41373314 | 103676 | 25:41416055 | 103678 | 25:41479611 | 103679 | 25:41496081 | 103682 | 25:41719279 |
| 103685 | 25:41758231 | 103695 | 25:42027128 | 103696 | 25:42031008 | 103703 | 25:42287133 | 103704 | 25:42290491 |
| 103705 | 25:42292572 | 103719 | 26:840412 | 103724 | 26:1046523 | 103733 | 26:1202224 | 103743 | 26:1527005 |
| 103751 | 26:1774152 | 103754 | 26:1837613 | 103755 | 26:1837679 | 103756 | 26:1837715 | 103789 | 26:2789785 |
| 103790 | 26:2795689 | 103791 | 26:2807861 | 103792 | 26:2809033 | 103793 | 26:2810402 | 103794 | 26:2810457 |
| 103795 | 26:2810725 | 103796 | 26:2811451 | 103797 | 26:2811491 | 103798 | 26:2811596 | 103799 | 26:2811603 |
| 103800 | 26:2811953 | 103801 | 26:2812213 | 103802 | 26:2812496 | 103803 | 26:2834801 | 103804 | 26:2836372 |
| 103805 | 26:2838470 | 103806 | 26:2839812 | 103807 | 26:2843878 | 103808 | 26:2847631 | 103809 | 26:2853005 |
| 103810 | 26:2869288 | 103812 | 26:2912970 | 103821 | 26:3233006 | 103825 | 26:3276739 | 103826 | 26:3276794 |
| 103827 | 26:3347635 | 103828 | 26:3347724 | 103853 | 26:3975478 | 103874 | 26:4592577 | 103891 | 26:4967710 |
| 103894 | 26:5039459 | 103904 | 26:5408963 | 103950 | 26:6941203 | 103952 | 26:6944017 | 103966 | 26:7431785 |
| 103968 | 26:7456216 | 103969 | 26:7484390 | 103970 | 26:7488314 | 103971 | 26:7495226 | 103972 | 26:7510638 |
| 103975 | 26:7591687 | 103979 | 26:7696472 | 103980 | 26:7696485 | 103981 | 26:7727559 | 103992 | 26:8027593 |
| 103993 | 26:8048518 | 104003 | 26:8204339 | 104016 | 26:8516699 | 104019 | 26:8538464 | 104026 | 26:8757164 |
| 104031 | 26:8943593 | 104032 | 26:8960699 | 104040 | 26:9137009 | 104055 | 26:9666870 | 104061 | 26:9801561 |
| 104078 | 26:10180467 | 104079 | 26:10180516 | 104080 | 26:10195205 | 104081 | 26:10195875 | 104082 | 26:10196016 |
| 104083 | 26:10197224 | 104084 | 26:10201006 | 104085 | 26:10203129 | 104086 | 26:10208363 | 104087 | 26:10212949 |
| 104088 | 26:10214107 | 104089 | 26:10221720 | 104093 | 26:10302490 | 104094 | 26:10305675 | 104107 | 26:10616738 |
| 104111 | 26:10731742 | 104129 | 26:11167367 | 104137 | 26:11511468 | 104146 | 26:11702998 | 104147 | 26:11703000 |
| 104148 | 26:11703003 | 104149 | 26:11703009 | 104150 | 26:11703013 | 104151 | 26:11703046 | 104152 | 26:11703059 |
| 104153 | 26:11703143 | 104171 | 26:12409311 | 104173 | 26:12493226 | 104174 | 26:12493314 | 104176 | 26:12576243 |
| 104179 | 26:12663124 | 104188 | 26:12857727 | 104190 | 26:12905617 | 104192 | 26:12936955 | 104193 | 26:13005236 |
| 104203 | 26:13185986 | 104206 | 26:13213326 | 104215 | 26:13237530 | 104216 | 26:13242646 | 104218 | 26:13253767 |
| 104221 | 26:13348439 | 104226 | 26:13439428 | 104229 | 26:13487765 | 104230 | 26:13487832 | 104233 | 26:13628978 |
| 104234 | 26:13632894 | 104236 | 26:13666033 | 104240 | 26:13818460 | 104243 | 26:13935413 | 104256 | 26:14204588 |
| 104257 | 26:14204609 | 104271 | 26:14670368 | 104272 | 26:14688221 | 104274 | 26:14729665 | 104278 | 26:14889689 |
| 104279 | 26:14890394 | 104280 | 26:14896553 | 104286 | 26:15033159 | 104299 | 26:15470094 | 104310 | 26:15856645 |
| 104311 | 26:15856778 | 104312 | 26:15865811 | 104313 | 26:15866192 | 104315 | 26:15867927 | 104316 | 26:15867943 |
| 104317 | 26:15876906 | 104340 | 26:16752906 | 104348 | 26:16926415 | 104349 | 26:16926501 | 104350 | 26:16951340 |
| 104353 | 26:17033041 | 104354 | 26:17041251 | 104358 | 26:17100094 | 104360 | 26:17127186 | 104361 | 26:17156417 |
| 104365 | 26:17197835 | 104369 | 26:17325459 | 104375 | 26:17482702 | 104376 | 26:17539828 | 104383 | 26:17628730 |
| 104384 | 26:17628780 | 104386 | 26:17685991 | 104387 | 26:17691480 | 104388 | 26:17691492 | 104390 | 26:17746468 |
| 104393 | 26:17781937 | 104398 | 26:17879952 | 104400 | 26:17928505 | 104402 | 26:17965085 | 104411 | 26:18209480 |
| 104412 | 26:18211289 | 104419 | 26:18355673 | 104423 | 26:18490628 | 104424 | 26:18494442 | 104425 | 26:18521176 |
| 104426 | 26:18528824 | 104433 | 26:18651237 | 104434 | 26:18695230 | 104435 | 26:18702491 | 104437 | 26:18728512 |
| 104438 | 26:18740541 | 104440 | 26:18782784 | 104443 | 26:18869630 | 104444 | 26:18885108 | 104445 | 26:18907964 |
| 104446 | 26:18929393 | 104450 | 26:18970293 | 104453 | 26:19009189 | 104464 | 26:19238259 | 104474 | 26:19438464 |
| 104475 | 26:19452547 | 104479 | 26:19494159 | 104480 | 26:19510054 | 104481 | 26:19547291 | 104483 | 26:19597530 |
| 104496 | 26:20104927 | 104497 | 26:20143627 | 104511 | 26:20594269 | 104529 | 26:21094234 | 104536 | 26:21239671 |
| 104537 | 26:21254361 | 104562 | 26:21784164 | 104564 | 26:21862417 | 104566 | 26:21918010 | 104568 | 26:21964449 |
| 104569 | 26:22002686 | 104571 | 26:22058857 | 104572 | 26:22063661 | 104578 | 26:22251251 | 104584 | 26:22554517 |
| 104585 | 26:22581259 | 104587 | 26:22606617 | 104589 | 26:22658743 | 104590 | 26:22659438 | 104611 | 26:23328557 |
| 104615 | 26:23398930 | 104618 | 26:23503177 | 104627 | 26:23741250 | 104633 | 26:24028517 | 104636 | 26:24127485 |
| 104637 | 26:24135884 | 104640 | 26:24188588 | 104643 | 26:24239524 | 104645 | 26:24286733 | 104646 | 26:24287516 |
| 104648 | 26:24310631 | 104657 | 26:24590237 | 104662 | 26:24630200 | 104665 | 26:24764833 | 104667 | 26:24781597 |
| 104669 | 26:24818702 | 104670 | 26:24819776 | 104671 | 26:24820679 | 104672 | 26:24823044 | 104673 | 26:24826317 |
| 104674 | 26:24829772 | 104675 | 26:24831082 | 104676 | 26:24831934 | 104678 | 26:24850967 | 104682 | 26:24867806 |
| 104683 | 26:24877750 | 104685 | 26:24920605 | 104705 | 26:25371117 | 104706 | 26:25377110 | 104710 | 26:25408794 |
| 104712 | 26:25438245 | 104720 | 26:25560669 | 104722 | 26:25586394 | 104723 | 26:25601218 | 104724 | 26:25624158 |
| 104725 | 26:25628586 | 104726 | 26:25632049 | 104727 | 26:25636772 | 104728 | 26:25637820 | 104729 | 26:25640830 |
| 104730 | 26:25643571 | 104731 | 26:25645847 | 104732 | 26:25646718 | 104733 | 26:25649902 | 104734 | 26:25653351 |
| 104735 | 26:25664984 | 104737 | 26:25690676 | 104740 | 26:25738398 | 104744 | 26:25884432 | 104746 | 26:25905471 |
| 104747 | 26:25923067 | 104749 | 26:25924666 | 104754 | 26:26022373 | 104757 | 26:26108845 | 104765 | 26:26209053 |
| 104766 | 26:26211205 | 104775 | 26:26461336 | 104792 | 26:26855141 | 104802 | 26:27069339 | 104820 | 26:27558387 |
| 104821 | 26:27572772 | 104832 | 26:27736462 | 104833 | 26:27736471 | 104834 | 26:27736612 | 104852 | 26:28197657 |
| 104871 | 26:28950952 | 104878 | 26:29088929 | 104882 | 26:29186399 | 104890 | 26:29513286 | 104910 | 26:29986030 |
| 104917 | 26:30197525 | 104918 | 26:30197564 | 104919 | 26:30197578 | 104920 | 26:30197681 | 104938 | 26:30889276 |
| 104939 | 26:30892846 | 104942 | 26:30950051 | 104956 | 26:31271680 | 104958 | 26:31311514 | 104959 | 26:31323087 |
| 104960 | 26:31350194 | 104962 | 26:31368850 | 104963 | 26:31379154 | 104965 | 26:31411730 | 104966 | 26:31483017 |
| 104979 | 26:31801170 | 104991 | 26:32178829 | 104992 | 26:32210509 | 105001 | 26:32484431 | 105004 | 26:32527484 |
| 105016 | 26:32691372 | 105019 | 26:32756815 | 105024 | 26:32879086 | 105034 | 26:33122620 | 105040 | 26:33282896 |
| 105041 | 26:33328268 | 105042 | 26:33359761 | 105043 | 26:33380765 | 105045 | 26:33400303 | 105050 | 26:33526762 |
| 105051 | 26:33530073 | 105053 | 26:33560198 | 105066 | 26:33837346 | 105069 | 26:33901952 | 105071 | 26:33914479 |
| 105072 | 26:33922739 | 105082 | 26:34087101 | 105083 | 26:34109859 | 105086 | 26:34125393 | 105087 | 26:34140428 |
| 105088 | 26:34145240 | 105089 | 26:34148000 | 105097 | 26:34318487 | 105099 | 26:34354045 | 105107 | 26:34633936 |
| 105111 | 26:34718279 | 105112 | 26:34746698 | 105113 | 26:34746782 | 105114 | 26:34746817 | 105115 | 26:34746843 |
| 105116 | 26:34746854 | 105117 | 26:34746898 | 105118 | 26:34755555 | 105122 | 26:34829732 | 105123 | 26:34832045 |
| 105129 | 26:35009461 | 105131 | 26:35045257 | 105136 | 26:35119651 | 105137 | 26:35152534 | 105147 | 26:35387482 |
| 105149 | 26:35446688 | 105150 | 26:35473524 | 105155 | 26:35703319 | 105156 | 26:35730641 | 105157 | 26:35730653 |
| 105158 | 26:35730692 | 105159 | 26:35730695 | 105164 | 26:35894255 | 105184 | 26:36442085 | 105185 | 26:36465890 |
| 105186 | 26:36466008 | 105199 | 26:36782486 | 105200 | 26:36868486 | 105201 | 26:36875653 | 105203 | 26:36881092 |
| 105204 | 26:36895816 | 105205 | 26:36899560 | 105206 | 26:36899693 | 105208 | 26:36955993 | 105209 | 26:36966986 |
| 105210 | 26:36968918 | 105211 | 26:36968980 | 105212 | 26:36969055 | 105220 | 26:37095749 | 105237 | 26:37812473 |
| 105240 | 26:37879370 | 105242 | 26:37908929 | 105248 | 26:38110713 | 105269 | 26:38674064 | 105275 | 26:38851055 |
| 105276 | 26:38854747 | 105277 | 26:38875810 | 105278 | 26:38877239 | 105279 | 26:38881457 | 105281 | 26:38992651 |
| 105284 | 26:39006604 | 105292 | 26:39098131 | 105297 | 26:39189961 | 105298 | 26:39196795 | 105299 | 26:39202461 |
| 105301 | 26:39305962 | 105304 | 26:39426186 | 105305 | 26:39426206 | 105306 | 26:39426264 | 105308 | 26:39485796 |
| 105311 | 26:39494577 | 105312 | 26:39507180 | 105314 | 26:39538069 | 105315 | 26:39573736 | 105323 | 26:39783330 |
| 105329 | 26:40032776 | 105330 | 26:40040946 | 105331 | 26:40070046 | 105342 | 26:40441942 | 105353 | 26:40786447 |
| 105360 | 26:41019385 | 105363 | 26:41076854 | 105364 | 26:41096640 | 105379 | 26:41390989 | 105396 | 26:41918819 |
| 105397 | 26:41926321 | 105402 | 26:42008210 | 105403 | 26:42024771 | 105405 | 26:42049951 | 105407 | 26:42090283 |
| 105408 | 26:42092905 | 105410 | 26:42109007 | 105415 | 26:42309356 | 105418 | 26:42330778 | 105420 | 26:42350916 |
| 105430 | 26:42642591 | 105432 | 26:42660447 | 105439 | 26:42757714 | 105449 | 26:43041112 | 105452 | 26:43062676 |
| 105469 | 26:43622892 | 105474 | 26:43740696 | 105478 | 26:43816442 | 105485 | 26:44064506 | 105488 | 26:44083273 |
| 105489 | 26:44083285 | 105490 | 26:44083315 | 105491 | 26:44083393 | 105492 | 26:44083680 | 105500 | 26:44255933 |
| 105503 | 26:44404294 | 105506 | 26:44481659 | 105510 | 26:44674003 | 105513 | 26:44723893 | 105523 | 26:45008042 |
| 105532 | 26:45209100 | 105536 | 26:45264287 | 105542 | 26:45324531 | 105550 | 26:45412608 | 105560 | 26:45540372 |
| 105561 | 26:45540404 | 105562 | 26:45540418 | 105563 | 26:45540427 | 105564 | 26:45542714 | 105567 | 26:45565711 |
| 105575 | 26:45729275 | 105587 | 26:46112041 | 105595 | 26:46315775 | 105596 | 26:46317103 | 105597 | 26:46317812 |
| 105598 | 26:46318923 | 105600 | 26:46353080 | 105601 | 26:46421801 | 105602 | 26:46452461 | 105604 | 26:46466580 |
| 105605 | 26:46471616 | 105606 | 26:46479016 | 105613 | 26:46559579 | 105631 | 26:47028707 | 105645 | 26:47333154 |
| 105646 | 26:47361635 | 105647 | 26:47427206 | 105651 | 26:47512133 | 105652 | 26:47512681 | 105658 | 26:47615883 |
| 105661 | 26:47650142 | 105667 | 26:47772970 | 105693 | 26:48408440 | 105698 | 26:48479152 | 105714 | 26:48865592 |
| 105715 | 26:48866736 | 105717 | 26:48968839 | 105719 | 26:49004191 | 105745 | 26:49515195 | 105749 | 26:49563646 |
| 105750 | 26:49563747 | 105794 | 26:50590249 | 105797 | 26:50709285 | 105799 | 26:50733543 | 105800 | 26:50733621 |
| 105805 | 26:50823585 | 105806 | 26:50825362 | 105807 | 26:50830075 | 105814 | 26:51009547 | 105815 | 26:51009628 |
| 105819 | 26:51127593 | 105821 | 26:51175800 | 105833 | 26:51422136 | 105844 | 26:51748308 | 105845 | 26:51784240 |
| 105846 | 26:51824417 | 105847 | 26:51837838 | 105848 | 26:51856701 | 105853 | 26:51990316 | 105861 | 27:504917 |
| 105862 | 27:504918 | 105863 | 27:504943 | 105865 | 27:622508 | 105875 | 27:1220407 | 105885 | 27:1285546 |
| 105888 | 27:1330181 | 105889 | 27:1355435 | 105890 | 27:1355462 | 105891 | 27:1362304 | 105892 | 27:1362959 |
| 105893 | 27:1375496 | 105894 | 27:1376218 | 105895 | 27:1382415 | 105896 | 27:1384981 | 105897 | 27:1385318 |
| 105898 | 27:1385323 | 105899 | 27:1385392 | 105900 | 27:1385425 | 105901 | 27:1385455 | 105903 | 27:1395995 |
| 105904 | 27:1401856 | 105905 | 27:1401865 | 105906 | 27:1405119 | 105907 | 27:1407822 | 105908 | 27:1453147 |
| 105911 | 27:1475170 | 105938 | 27:2200746 | 105939 | 27:2232971 | 105946 | 27:2375493 | 105954 | 27:2508576 |
| 105976 | 27:2988336 | 106001 | 27:3776760 | 106002 | 27:3787074 | 106003 | 27:3788517 | 106004 | 27:3790934 |
| 106005 | 27:3805789 | 106027 | 27:4234197 | 106047 | 27:4820720 | 106048 | 27:4843559 | 106050 | 27:4942987 |
| 106051 | 27:4946073 | 106052 | 27:4962502 | 106053 | 27:4964043 | 106057 | 27:5085626 | 106060 | 27:5098545 |
| 106061 | 27:5158253 | 106069 | 27:5391087 | 106070 | 27:5392011 | 106071 | 27:5395069 | 106072 | 27:5396567 |
| 106073 | 27:5399954 | 106074 | 27:5401556 | 106075 | 27:5402389 | 106076 | 27:5403342 | 106077 | 27:5404401 |
| 106079 | 27:5418944 | 106080 | 27:5421637 | 106081 | 27:5428881 | 106082 | 27:5430538 | 106084 | 27:5470087 |
| 106085 | 27:5478901 | 106089 | 27:5582204 | 106090 | 27:5587883 | 106091 | 27:5611632 | 106097 | 27:5792112 |
| 106100 | 27:5827416 | 106104 | 27:5974973 | 106105 | 27:5988748 | 106107 | 27:6025859 | 106109 | 27:6032871 |
| 106111 | 27:6060484 | 106114 | 27:6087111 | 106140 | 27:7631148 | 106142 | 27:7653356 | 106148 | 27:7733608 |
| 106150 | 27:7750098 | 106151 | 27:7770345 | 106153 | 27:7791683 | 106154 | 27:7807058 | 106155 | 27:7813201 |
| 106160 | 27:7920370 | 106162 | 27:7920432 | 106163 | 27:7920451 | 106167 | 27:7958336 | 106170 | 27:7975782 |
| 106171 | 27:7980889 | 106175 | 27:8132488 | 106177 | 27:8191553 | 106180 | 27:8289670 | 106182 | 27:8359908 |
| 106187 | 27:8470036 | 106188 | 27:8476834 | 106192 | 27:8575128 | 106194 | 27:8612967 | 106239 | 27:9145723 |
| 106244 | 27:9202230 | 106245 | 27:9202257 | 106246 | 27:9202263 | 106247 | 27:9202295 | 106254 | 27:9488574 |
| 106307 | 27:11255396 | 106308 | 27:11262348 | 106325 | 27:11669357 | 106327 | 27:11736207 | 106342 | 27:12235101 |
| 106344 | 27:12270834 | 106370 | 27:12976567 | 106378 | 27:13162684 | 106387 | 27:13319931 | 106393 | 27:13521583 |
| 106406 | 27:13703268 | 106409 | 27:13763652 | 106410 | 27:13769140 | 106417 | 27:13864569 | 106454 | 27:14058907 |
| 106455 | 27:14089539 | 106464 | 27:14337753 | 106473 | 27:14505664 | 106477 | 27:14619598 | 106500 | 27:15114953 |
| 106505 | 27:15209444 | 106512 | 27:15342336 | 106514 | 27:15374174 | 106529 | 27:15616842 | 106531 | 27:15679545 |
| 106532 | 27:15679907 | 106533 | 27:15679986 | 106534 | 27:15679987 | 106536 | 27:15724630 | 106552 | 27:16091144 |
| 106565 | 27:16231107 | 106566 | 27:16231139 | 106568 | 27:16250819 | 106569 | 27:16262844 | 106570 | 27:16289197 |
| 106571 | 27:16289212 | 106573 | 27:16310423 | 106575 | 27:16311908 | 106576 | 27:16311992 | 106582 | 27:16448657 |
| 106585 | 27:16475268 | 106588 | 27:16527145 | 106589 | 27:16527632 | 106591 | 27:16545436 | 106593 | 27:16626449 |
| 106604 | 27:16876243 | 106614 | 27:17114420 | 106635 | 27:17756331 | 106639 | 27:17825429 | 106647 | 27:17900781 |
| 106682 | 27:18743674 | 106704 | 27:19184326 | 106707 | 27:19226515 | 106712 | 27:19413103 | 106722 | 27:19615386 |
| 106724 | 27:19628648 | 106725 | 27:19638432 | 106726 | 27:19643687 | 106727 | 27:19643703 | 106729 | 27:19656115 |
| 106732 | 27:19709555 | 106733 | 27:19715294 | 106734 | 27:19715322 | 106735 | 27:19743997 | 106737 | 27:19759721 |
| 106743 | 27:19916100 | 106758 | 27:20275887 | 106761 | 27:20403248 | 106778 | 27:21028770 | 106796 | 27:21603965 |
| 106803 | 27:21736051 | 106819 | 27:21968621 | 106820 | 27:21988370 | 106821 | 27:21988471 | 106822 | 27:22006706 |
| 106835 | 27:22555224 | 106865 | 27:23217717 | 106866 | 27:23219768 | 106867 | 27:23235008 | 106869 | 27:23291834 |
| 106883 | 27:23591760 | 106888 | 27:23763903 | 106891 | 27:23820540 | 106896 | 27:23900777 | 106900 | 27:23960140 |
| 106901 | 27:23969325 | 106904 | 27:23997295 | 106907 | 27:24130313 | 106910 | 27:24151993 | 106911 | 27:24152004 |
| 106913 | 27:24203304 | 106914 | 27:24203421 | 106916 | 27:24222816 | 106929 | 27:24521008 | 106938 | 27:24725230 |
| 106948 | 27:24839161 | 106949 | 27:24839261 | 106965 | 27:25252926 | 106969 | 27:25328242 | 106974 | 27:25416389 |
| 106981 | 27:25562534 | 106982 | 27:25566427 | 106986 | 27:25728403 | 106996 | 27:26089588 | 107002 | 27:26265901 |
| 107005 | 27:26306516 | 107009 | 27:26385003 | 107019 | 27:26490500 | 107025 | 27:26581172 | 107026 | 27:26586255 |
| 107027 | 27:26593729 | 107039 | 27:26832000 | 107040 | 27:26846204 | 107041 | 27:26878354 | 107042 | 27:26886076 |
| 107043 | 27:26894469 | 107044 | 27:26904559 | 107045 | 27:26906127 | 107047 | 27:26924212 | 107061 | 27:27222640 |
| 107062 | 27:27223927 | 107063 | 27:27223929 | 107064 | 27:27224038 | 107065 | 27:27224048 | 107066 | 27:27224849 |
| 107067 | 27:27224850 | 107081 | 27:27637602 | 107096 | 27:27970577 | 107104 | 27:28171888 | 107130 | 27:28946541 |
| 107149 | 27:29252315 | 107151 | 27:29278521 | 107153 | 27:29296483 | 107156 | 27:29312772 | 107158 | 27:29327883 |
| 107159 | 27:29327914 | 107161 | 27:29327998 | 107188 | 27:30202829 | 107190 | 27:30202912 | 107191 | 27:30226393 |
| 107192 | 27:30244634 | 107221 | 27:31002073 | 107222 | 27:31002095 | 107223 | 27:31002129 | 107230 | 27:31078802 |
| 107240 | 27:31274065 | 107263 | 27:31729965 | 107268 | 27:31841660 | 107269 | 27:31841704 | 107273 | 27:31897740 |
| 107296 | 27:32226765 | 107297 | 27:32254312 | 107299 | 27:32265770 | 107301 | 27:32268130 | 107303 | 27:32276493 |
| 107311 | 27:32518470 | 107312 | 27:32518472 | 107313 | 27:32518552 | 107317 | 27:32583194 | 107321 | 27:32727516 |
| 107322 | 27:32727582 | 107323 | 27:32727603 | 107324 | 27:32727620 | 107327 | 27:32778474 | 107329 | 27:32793041 |
| 107330 | 27:32802126 | 107340 | 27:33001000 | 107341 | 27:33009928 | 107348 | 27:33086268 | 107356 | 27:33233127 |
| 107358 | 27:33265366 | 107359 | 27:33272590 | 107363 | 27:33421612 | 107367 | 27:33629831 | 107368 | 27:33633260 |
| 107374 | 27:33872186 | 107375 | 27:33879901 | 107378 | 27:33913097 | 107382 | 27:34127235 | 107399 | 27:34635911 |
| 107400 | 27:34637563 | 107403 | 27:34752720 | 107413 | 27:34981815 | 107414 | 27:35014810 | 107415 | 27:35015975 |
| 107416 | 27:35016903 | 107417 | 27:35018328 | 107418 | 27:35019984 | 107419 | 27:35021367 | 107420 | 27:35026180 |
| 107421 | 27:35032183 | 107422 | 27:35042729 | 107424 | 27:35045715 | 107425 | 27:35047820 | 107426 | 27:35052712 |
| 107427 | 27:35053785 | 107428 | 27:35054739 | 107429 | 27:35058765 | 107430 | 27:35060134 | 107431 | 27:35064362 |
| 107432 | 27:35065072 | 107434 | 27:35072059 | 107435 | 27:35075103 | 107436 | 27:35079318 | 107437 | 27:35080042 |
| 107438 | 27:35098005 | 107440 | 27:35103784 | 107441 | 27:35121417 | 107442 | 27:35122313 | 107443 | 27:35128376 |
| 107444 | 27:35138175 | 107446 | 27:35184790 | 107470 | 27:35555630 | 107475 | 27:35599360 | 107484 | 27:35782254 |
| 107486 | 27:35824524 | 107487 | 27:35824606 | 107497 | 27:35975385 | 107498 | 27:35978336 | 107502 | 27:36138112 |
| 107506 | 27:36216370 | 107522 | 27:36576641 | 107531 | 27:36751396 | 107535 | 27:36906500 | 107539 | 27:37106071 |
| 107540 | 27:37127877 | 107542 | 27:37172084 | 107543 | 27:37192870 | 107544 | 27:37195004 | 107549 | 27:37274132 |
| 107550 | 27:37275861 | 107552 | 27:37293040 | 107553 | 27:37297500 | 107560 | 27:37374389 | 107566 | 27:37486971 |
| 107567 | 27:37493520 | 107568 | 27:37521766 | 107571 | 27:37573787 | 107572 | 27:37620840 | 107575 | 27:37630299 |
| 107576 | 27:37639061 | 107583 | 27:37746356 | 107590 | 27:37827637 | 107592 | 27:37864645 | 107603 | 27:38151557 |
| 107604 | 27:38168061 | 107611 | 27:38327204 | 107616 | 27:38432520 | 107620 | 27:38491615 | 107636 | 27:38861489 |
| 107643 | 27:39015604 | 107644 | 27:39015763 | 107645 | 27:39015976 | 107668 | 27:39667527 | 107669 | 27:39668431 |
| 107673 | 27:39780803 | 107681 | 27:40004933 | 107691 | 27:40356156 | 107692 | 27:40357028 | 107694 | 27:40414746 |
| 107702 | 27:40674026 | 107709 | 27:40868181 | 107716 | 27:41044067 | 107724 | 27:41178088 | 107736 | 27:41583577 |
| 107742 | 27:41702146 | 107752 | 27:41886684 | 107760 | 27:42166403 | 107763 | 27:42228982 | 107766 | 27:42242635 |
| 107774 | 27:42427599 | 107775 | 27:42462522 | 107777 | 27:42487443 | 107784 | 27:42794556 | 107815 | 27:43428164 |
| 107833 | 27:43823427 | 107837 | 27:43863511 | 107839 | 27:43908553 | 107842 | 27:43927325 | 107848 | 27:43987089 |
| 107850 | 27:43995458 | 107858 | 27:44200677 | 107870 | 27:44375041 | 107871 | 27:44412495 | 107880 | 27:44543531 |
| 107884 | 27:44629778 | 107927 | 28:479125 | 107928 | 28:504407 | 107931 | 28:692680 | 107932 | 28:704706 |
| 107933 | 28:704958 | 107949 | 28:1196781 | 107951 | 28:1230989 | 107952 | 28:1234367 | 107954 | 28:1243626 |
| 107955 | 28:1243641 | 107956 | 28:1243692 | 107959 | 28:1320241 | 107962 | 28:1399889 | 107967 | 28:1487331 |
| 107968 | 28:1489641 | 107984 | 28:1783556 | 107986 | 28:1818831 | 107989 | 28:1840156 | 107993 | 28:1924353 |
| 107995 | 28:1964866 | 107997 | 28:2032083 | 108002 | 28:2144906 | 108003 | 28:2162175 | 108010 | 28:2343692 |
| 108021 | 28:2709799 | 108022 | 28:2730701 | 108034 | 28:3272910 | 108037 | 28:3476063 | 108038 | 28:3548826 |
| 108039 | 28:3590983 | 108045 | 28:3973141 | 108046 | 28:3973211 | 108047 | 28:3984834 | 108052 | 28:4431180 |
| 108056 | 28:4631548 | 108060 | 28:4701678 | 108068 | 28:4907755 | 108071 | 28:5014037 | 108080 | 28:5220057 |
| 108081 | 28:5220146 | 108082 | 28:5220186 | 108085 | 28:5245742 | 108086 | 28:5247877 | 108088 | 28:5253360 |
| 108101 | 28:5630191 | 108107 | 28:5815635 | 108108 | 28:5833150 | 108110 | 28:5839078 | 108111 | 28:5870781 |
| 108112 | 28:5870881 | 108113 | 28:5873548 | 108114 | 28:5873612 | 108115 | 28:5873655 | 108116 | 28:5874287 |
| 108119 | 28:5903348 | 108146 | 28:6691792 | 108154 | 28:6866814 | 108155 | 28:6866847 | 108156 | 28:6866914 |
| 108169 | 28:7257360 | 108172 | 28:7304728 | 108173 | 28:7321818 | 108174 | 28:7339237 | 108175 | 28:7378556 |
| 108176 | 28:7415736 | 108184 | 28:7555026 | 108189 | 28:7760692 | 108191 | 28:7786515 | 108192 | 28:7813529 |
| 108208 | 28:8105292 | 108209 | 28:8125888 | 108214 | 28:8201519 | 108219 | 28:8331969 | 108225 | 28:8547431 |
| 108230 | 28:8650652 | 108234 | 28:8698842 | 108235 | 28:8707863 | 108241 | 28:8843955 | 108246 | 28:9025349 |
| 108253 | 28:9100351 | 108258 | 28:9269631 | 108261 | 28:9336102 | 108263 | 28:9386021 | 108269 | 28:9586664 |
| 108280 | 28:9807554 | 108284 | 28:9913957 | 108285 | 28:9914017 | 108286 | 28:9915219 | 108287 | 28:9915233 |
| 108301 | 28:10277520 | 108359 | 28:12225835 | 108375 | 28:12371401 | 108385 | 28:12623784 | 108386 | 28:12623831 |
| 108387 | 28:12623855 | 108404 | 28:13022825 | 108411 | 28:13252341 | 108434 | 28:13950890 | 108442 | 28:14104824 |
| 108445 | 28:14144978 | 108446 | 28:14157633 | 108448 | 28:14181805 | 108449 | 28:14204540 | 108451 | 28:14219987 |
| 108454 | 28:14342309 | 108456 | 28:14415424 | 108457 | 28:14415472 | 108464 | 28:14547089 | 108466 | 28:14552705 |
| 108477 | 28:14925580 | 108480 | 28:14960497 | 108483 | 28:15003137 | 108495 | 28:15386201 | 108496 | 28:15392967 |
| 108501 | 28:15541195 | 108502 | 28:15543708 | 108504 | 28:15562551 | 108515 | 28:15866947 | 108516 | 28:15875883 |
| 108518 | 28:15913989 | 108519 | 28:15913997 | 108520 | 28:15914061 | 108521 | 28:15914091 | 108522 | 28:15942493 |
| 108523 | 28:15972055 | 108528 | 28:16010478 | 108531 | 28:16039905 | 108534 | 28:16124447 | 108535 | 28:16124496 |
| 108544 | 28:16335973 | 108546 | 28:16387453 | 108548 | 28:16407912 | 108552 | 28:16520465 | 108553 | 28:16588164 |
| 108556 | 28:16644285 | 108557 | 28:16644893 | 108558 | 28:16648829 | 108560 | 28:16661175 | 108561 | 28:16661840 |
| 108563 | 28:16692080 | 108566 | 28:16767636 | 108567 | 28:16773322 | 108569 | 28:16818843 | 108573 | 28:16930270 |
| 108584 | 28:17215906 | 108589 | 28:17282866 | 108590 | 28:17285666 | 108591 | 28:17285711 | 108609 | 28:17588476 |
| 108616 | 28:17835910 | 108625 | 28:18019905 | 108626 | 28:18019907 | 108627 | 28:18019913 | 108628 | 28:18019925 |
| 108634 | 28:18086730 | 108635 | 28:18107994 | 108648 | 28:18485866 | 108662 | 28:18937069 | 108666 | 28:19041963 |
| 108669 | 28:19127485 | 108675 | 28:19229652 | 108682 | 28:19534804 | 108685 | 28:19583995 | 108686 | 28:19673238 |
| 108694 | 28:19774362 | 108718 | 28:20415689 | 108756 | 28:21574143 | 108788 | 28:22399855 | 108789 | 28:22479622 |
| 108792 | 28:22620494 | 108793 | 28:22620545 | 108794 | 28:22620582 | 108800 | 28:22847773 | 108806 | 28:23007374 |
| 108807 | 28:23007384 | 108809 | 28:23049818 | 108815 | 28:23173525 | 108816 | 28:23177219 | 108822 | 28:23292046 |
| 108824 | 28:23296757 | 108829 | 28:23385467 | 108830 | 28:23385512 | 108852 | 28:24001450 | 108860 | 28:24160079 |
| 108861 | 28:24160811 | 108868 | 28:24242145 | 108869 | 28:24253020 | 108884 | 28:24623959 | 108892 | 28:24802411 |
| 108893 | 28:24802550 | 108894 | 28:24802551 | 108897 | 28:24885896 | 108900 | 28:24934653 | 108904 | 28:25064734 |
| 108906 | 28:25090839 | 108909 | 28:25139537 | 108918 | 28:25385010 | 108919 | 28:25408853 | 108920 | 28:25415208 |
| 108928 | 28:25614547 | 108929 | 28:25625624 | 108930 | 28:25625649 | 108931 | 28:25625667 | 108932 | 28:25626954 |
| 108934 | 28:25731593 | 108937 | 28:25808647 | 108942 | 28:25947218 | 108948 | 28:26030383 | 108949 | 28:26036200 |
| 108957 | 28:26155221 | 108959 | 28:26180213 | 108960 | 28:26181587 | 108961 | 28:26181622 | 108962 | 28:26181655 |
| 108963 | 28:26185280 | 108964 | 28:26185344 | 108965 | 28:26185376 | 108966 | 28:26186082 | 108967 | 28:26186104 |
| 108968 | 28:26186116 | 108969 | 28:26186150 | 108981 | 28:26504153 | 108988 | 28:26606263 | 108992 | 28:26681661 |
| 108995 | 28:26740880 | 108996 | 28:26752280 | 108999 | 28:26814555 | 109008 | 28:27066599 | 109009 | 28:27068380 |
| 109010 | 28:27070779 | 109017 | 28:27146373 | 109023 | 28:27317961 | 109033 | 28:27518059 | 109034 | 28:27566739 |
| 109041 | 28:27704605 | 109045 | 28:27851635 | 109055 | 28:27937188 | 109056 | 28:27937338 | 109057 | 28:27937971 |
| 109060 | 28:27941915 | 109061 | 28:27941916 | 109062 | 28:27941932 | 109063 | 28:27943776 | 109064 | 28:27943868 |
| 109065 | 28:27943901 | 109066 | 28:27943916 | 109068 | 28:27948642 | 109069 | 28:27948643 | 109071 | 28:27954047 |
| 109078 | 28:28109673 | 109086 | 28:28192038 | 109089 | 28:28290243 | 109090 | 28:28290255 | 109091 | 28:28290934 |
| 109092 | 28:28290955 | 109093 | 28:28290999 | 109095 | 28:28313393 | 109100 | 28:28407860 | 109106 | 28:28646964 |
| 109109 | 28:28722550 | 109115 | 28:28920066 | 109116 | 28:28920811 | 109117 | 28:28920813 | 109130 | 28:29172597 |
| 109133 | 28:29210804 | 109134 | 28:29213687 | 109135 | 28:29236992 | 109137 | 28:29298781 | 109151 | 28:29704456 |
| 109156 | 28:29735862 | 109157 | 28:29737399 | 109159 | 28:29776190 | 109160 | 28:29777653 | 109162 | 28:29841383 |
| 109165 | 28:29914673 | 109171 | 28:30046702 | 109178 | 28:30226392 | 109188 | 28:30648480 | 109193 | 28:30730091 |
| 109194 | 28:30770433 | 109196 | 28:30824174 | 109200 | 28:30869446 | 109203 | 28:30999414 | 109205 | 28:31017300 |
| 109206 | 28:31042068 | 109210 | 28:31093235 | 109218 | 28:31386148 | 109224 | 28:31501902 | 109226 | 28:31528266 |
| 109227 | 28:31531949 | 109234 | 28:31783641 | 109253 | 28:32102216 | 109254 | 28:32102950 | 109255 | 28:32104334 |
| 109256 | 28:32119157 | 109257 | 28:32119371 | 109258 | 28:32122100 | 109259 | 28:32141403 | 109266 | 28:32322725 |
| 109272 | 28:32385967 | 109273 | 28:32392342 | 109306 | 28:32904173 | 109309 | 28:32960277 | 109310 | 28:32997331 |
| 109314 | 28:33041333 | 109320 | 28:33158395 | 109321 | 28:33186798 | 109324 | 28:33225867 | 109326 | 28:33239306 |
| 109330 | 28:33329500 | 109331 | 28:33342708 | 109333 | 28:33368759 | 109341 | 28:33551708 | 109342 | 28:33555527 |
| 109345 | 28:33662006 | 109352 | 28:33744728 | 109354 | 28:33822791 | 109355 | 28:33823084 | 109364 | 28:34010065 |
| 109367 | 28:34062194 | 109373 | 28:34243304 | 109374 | 28:34254734 | 109379 | 28:34333090 | 109382 | 28:34496870 |
| 109384 | 28:34523399 | 109392 | 28:34603788 | 109403 | 28:34778574 | 109404 | 28:34786048 | 109408 | 28:34828202 |
| 109410 | 28:34853555 | 109412 | 28:34912784 | 109419 | 28:34993324 | 109421 | 28:35011293 | 109424 | 28:35125823 |
| 109425 | 28:35132199 | 109426 | 28:35132247 | 109432 | 28:35222450 | 109435 | 28:35291319 | 109441 | 28:35644657 |
| 109443 | 28:35671648 | 109444 | 28:35672175 | 109446 | 28:35680732 | 109447 | 28:35683234 | 109448 | 28:35685429 |
| 109453 | 28:35805891 | 109456 | 28:35837667 | 109459 | 28:35923596 | 109462 | 28:36015548 | 109476 | 28:36205983 |
| 109488 | 28:36587691 | 109525 | 28:37533725 | 109541 | 28:37721207 | 109600 | 28:39163650 | 109601 | 28:39164865 |
| 109602 | 28:39164902 | 109603 | 28:39164949 | 109604 | 28:39167717 | 109605 | 28:39167797 | 109606 | 28:39167807 |
| 109607 | 28:39167808 | 109608 | 28:39167836 | 109609 | 28:39167853 | 109614 | 28:39266326 | 109616 | 28:39290663 |
| 109618 | 28:39316523 | 109635 | 28:39818069 | 109637 | 28:39868243 | 109664 | 28:40526877 | 109665 | 28:40526886 |
| 109666 | 28:40547129 | 109668 | 28:40606521 | 109669 | 28:40609071 | 109670 | 28:40613418 | 109673 | 28:40641661 |
| 109674 | 28:40644608 | 109675 | 28:40647540 | 109676 | 28:40648502 | 109677 | 28:40649183 | 109678 | 28:40650128 |
| 109679 | 28:40650866 | 109680 | 28:40651548 | 109681 | 28:40652228 | 109682 | 28:40653623 | 109683 | 28:40654155 |
| 109684 | 28:40654919 | 109685 | 28:40655651 | 109686 | 28:40656367 | 109687 | 28:40658031 | 109688 | 28:40659095 |
| 109690 | 28:40679353 | 109691 | 28:40683789 | 109692 | 28:40684338 | 109693 | 28:40685176 | 109694 | 28:40685911 |
| 109695 | 28:40687468 | 109696 | 28:40688010 | 109697 | 28:40688917 | 109698 | 28:40690412 | 109699 | 28:40691166 |
| 109700 | 28:40692798 | 109701 | 28:40693384 | 109702 | 28:40694350 | 109703 | 28:40695394 | 109704 | 28:40697087 |
| 109705 | 28:40698875 | 109706 | 28:40700189 | 109707 | 28:40701565 | 109708 | 28:40707053 | 109709 | 28:40707560 |
| 109710 | 28:40708330 | 109711 | 28:40709943 | 109712 | 28:40711617 | 109713 | 28:40714408 | 109714 | 28:40716186 |
| 109715 | 28:40717895 | 109716 | 28:40718746 | 109717 | 28:40719687 | 109718 | 28:40720636 | 109719 | 28:40721303 |
| 109720 | 28:40722936 | 109721 | 28:40723874 | 109722 | 28:40724503 | 109723 | 28:40725696 | 109724 | 28:40726977 |
| 109733 | 28:40857491 | 109740 | 28:40977297 | 109742 | 28:41002895 | 109747 | 28:41077744 | 109754 | 28:41244211 |
| 109757 | 28:41283427 | 109759 | 28:41333995 | 109772 | 28:41565674 | 109780 | 28:41666097 | 109781 | 28:41666186 |
| 109783 | 28:41724320 | 109784 | 28:41728249 | 109786 | 28:41731568 | 109789 | 28:41757290 | 109796 | 28:41885934 |
| 109799 | 28:41913803 | 109800 | 28:41952391 | 109803 | 28:41980728 | 109804 | 28:41986845 | 109806 | 28:41989663 |
| 109808 | 28:42038658 | 109811 | 28:42092356 | 109823 | 28:42424892 | 109831 | 28:42588414 | 109835 | 28:42648031 |
| 109840 | 28:42816073 | 109841 | 28:42822333 | 109845 | 28:42894677 | 109852 | 28:43061595 | 109854 | 28:43097491 |
| 109856 | 28:43111417 | 109857 | 28:43111489 | 109858 | 28:43111570 | 109859 | 28:43112699 | 109860 | 28:43112752 |
| 109861 | 28:43112807 | 109862 | 28:43112817 | 109863 | 28:43112858 | 109865 | 28:43140781 | 109870 | 28:43230125 |
| 109873 | 28:43258213 | 109880 | 28:43420442 | 109883 | 28:43482124 | 109885 | 28:43501640 | 109888 | 28:43538406 |
| 109890 | 28:43579200 | 109894 | 28:43651140 | 109906 | 28:44028372 | 109907 | 28:44030407 | 109908 | 28:44031081 |
| 109909 | 28:44038721 | 109910 | 28:44039225 | 109911 | 28:44039304 | 109917 | 28:44143344 | 109918 | 28:44157666 |
| 109927 | 28:44264765 | 109934 | 28:44453518 | 109941 | 28:44687207 | 109949 | 28:44852965 | 109969 | 28:45172059 |
| 109975 | 28:45281713 | 109979 | 28:45314101 | 109982 | 28:45416826 | 109985 | 28:45476974 | 109991 | 28:45579104 |
| 110000 | 28:45713679 | 110003 | 28:45771532 | 110006 | 28:45847568 | 110014 | 29:612126 | 110018 | 29:699249 |
| 110020 | 29:699337 | 110021 | 29:699732 | 110022 | 29:699767 | 110028 | 29:750365 | 110042 | 29:1219671 |
| 110046 | 29:1273559 | 110049 | 29:1315459 | 110056 | 29:1429261 | 110058 | 29:1499682 | 110059 | 29:1512567 |
| 110060 | 29:1512626 | 110072 | 29:1772440 | 110084 | 29:2136150 | 110093 | 29:2325537 | 110099 | 29:2380491 |
| 110118 | 29:2594653 | 110119 | 29:2620380 | 110135 | 29:2994930 | 110146 | 29:3175555 | 110155 | 29:3406656 |
| 110169 | 29:3717232 | 110190 | 29:4246237 | 110198 | 29:4406319 | 110206 | 29:4643160 | 110224 | 29:5107639 |
| 110259 | 29:6283155 | 110260 | 29:6283208 | 110277 | 29:6779650 | 110286 | 29:7101326 | 110293 | 29:7255100 |
| 110299 | 29:7426428 | 110339 | 29:8601758 | 110348 | 29:8772433 | 110350 | 29:8793187 | 110352 | 29:8821458 |
| 110355 | 29:8883493 | 110357 | 29:8926820 | 110361 | 29:8998775 | 110363 | 29:9022834 | 110364 | 29:9022891 |
| 110369 | 29:9102521 | 110378 | 29:9365821 | 110386 | 29:9490114 | 110403 | 29:9846120 | 110406 | 29:9882247 |
| 110423 | 29:10340072 | 110447 | 29:10979545 | 110460 | 29:11204386 | 110463 | 29:11241638 | 110465 | 29:11260132 |
| 110467 | 29:11341144 | 110478 | 29:11709562 | 110484 | 29:11839933 | 110487 | 29:11891707 | 110491 | 29:11952953 |
| 110492 | 29:11977550 | 110495 | 29:12018235 | 110498 | 29:12084416 | 110500 | 29:12114717 | 110501 | 29:12138482 |
| 110504 | 29:12182249 | 110511 | 29:12394797 | 110516 | 29:12538136 | 110519 | 29:12589589 | 110521 | 29:12619588 |
| 110522 | 29:12622700 | 110541 | 29:12987411 | 110558 | 29:13574420 | 110559 | 29:13574513 | 110568 | 29:13821500 |
| 110586 | 29:14373492 | 110587 | 29:14374640 | 110596 | 29:14741850 | 110597 | 29:14741861 | 110598 | 29:14741873 |
| 110599 | 29:14741887 | 110600 | 29:14741921 | 110608 | 29:14900262 | 110617 | 29:15134872 | 110623 | 29:15304699 |
| 110626 | 29:15395863 | 110646 | 29:15811607 | 110666 | 29:16464456 | 110667 | 29:16485296 | 110690 | 29:16964102 |
| 110693 | 29:16992973 | 110697 | 29:17055985 | 110703 | 29:17207473 | 110709 | 29:17442223 | 110711 | 29:17505934 |
| 110712 | 29:17540650 | 110718 | 29:17700379 | 110733 | 29:18393448 | 110739 | 29:18612768 | 110783 | 29:20127489 |
| 110797 | 29:20506894 | 110799 | 29:20644568 | 110800 | 29:20651157 | 110801 | 29:20673987 | 110802 | 29:20674048 |
| 110824 | 29:21228889 | 110826 | 29:21315170 | 110830 | 29:21392121 | 110831 | 29:21392184 | 110832 | 29:21392189 |
| 110843 | 29:21600071 | 110861 | 29:22072294 | 110864 | 29:22180877 | 110866 | 29:22297264 | 110868 | 29:22344163 |
| 110881 | 29:22760861 | 110885 | 29:22836272 | 110904 | 29:23318559 | 110909 | 29:23357907 | 110918 | 29:23560620 |
| 110920 | 29:23583982 | 110923 | 29:23628287 | 110926 | 29:23661992 | 110928 | 29:23731784 | 110932 | 29:23850587 |
| 110934 | 29:23937388 | 110945 | 29:24158412 | 110950 | 29:24258248 | 110952 | 29:24330084 | 110953 | 29:24354233 |
| 110954 | 29:24362811 | 110959 | 29:24490616 | 110969 | 29:24840874 | 110978 | 29:25015460 | 110980 | 29:25071999 |
| 110988 | 29:25205531 | 110990 | 29:25222326 | 110995 | 29:25305438 | 110997 | 29:25371765 | 110998 | 29:25438251 |
| 111015 | 29:25903150 | 111016 | 29:25908734 | 111019 | 29:26070069 | 111021 | 29:26105651 | 111038 | 29:26549594 |
| 111046 | 29:26946250 | 111048 | 29:26946336 | 111049 | 29:26946383 | 111050 | 29:26947201 | 111051 | 29:26947224 |
| 111052 | 29:26962161 | 111067 | 29:27537716 | 111068 | 29:27537760 | 111069 | 29:27538148 | 111072 | 29:27595444 |
| 111078 | 29:27728132 | 111083 | 29:27790794 | 111084 | 29:27808076 | 111085 | 29:27808486 | 111087 | 29:27823998 |
| 111088 | 29:27824016 | 111089 | 29:27824039 | 111090 | 29:27824126 | 111095 | 29:28026650 | 111097 | 29:28100144 |
| 111104 | 29:28218734 | 111105 | 29:28226254 | 111106 | 29:28228229 | 111108 | 29:28251283 | 111111 | 29:28278699 |
| 111117 | 29:28370448 | 111118 | 29:28430575 | 111122 | 29:28519839 | 111124 | 29:28574787 | 111126 | 29:28655873 |
| 111129 | 29:28715263 | 111134 | 29:28815692 | 111136 | 29:28826292 | 111159 | 29:29397513 | 111160 | 29:29397515 |
| 111163 | 29:29461701 | 111164 | 29:29472010 | 111168 | 29:29555287 | 111171 | 29:29629619 | 111172 | 29:29649318 |
| 111173 | 29:29653990 | 111174 | 29:29665600 | 111176 | 29:29676297 | 111177 | 29:29682536 | 111178 | 29:29690811 |
| 111183 | 29:29755215 | 111184 | 29:29755216 | 111185 | 29:29755261 | 111186 | 29:29755320 | 111187 | 29:29756679 |
| 111188 | 29:29756692 | 111196 | 29:29928478 | 111197 | 29:29928524 | 111198 | 29:29928553 | 111199 | 29:29928591 |
| 111200 | 29:29928636 | 111201 | 29:29928675 | 111202 | 29:29928677 | 111214 | 29:30201851 | 111217 | 29:30294652 |
| 111239 | 29:30759911 | 111240 | 29:30759984 | 111253 | 29:31124828 | 111264 | 29:31355454 | 111265 | 29:31355522 |
| 111271 | 29:31455230 | 111273 | 29:31488540 | 111280 | 29:31676141 | 111285 | 29:31801460 | 111286 | 29:31801542 |
| 111288 | 29:31845002 | 111289 | 29:31845960 | 111304 | 29:32253717 | 111311 | 29:32397724 | 111316 | 29:32544942 |
| 111319 | 29:32611173 | 111321 | 29:32693113 | 111323 | 29:32753972 | 111324 | 29:32754754 | 111325 | 29:32764982 |
| 111326 | 29:32765663 | 111327 | 29:32766346 | 111330 | 29:32842956 | 111343 | 29:33120665 | 111357 | 29:33560766 |
| 111363 | 29:33704537 | 111370 | 29:33824948 | 111397 | 29:34433856 | 111414 | 29:34688201 | 111420 | 29:34751704 |
| 111421 | 29:34751748 | 111431 | 29:34985663 | 111446 | 29:35373548 | 111447 | 29:35393862 | 111448 | 29:35393931 |
| 111453 | 29:35506238 | 111460 | 29:35663595 | 111462 | 29:35697578 | 111473 | 29:35864204 | 111476 | 29:35918743 |
| 111485 | 29:36106383 | 111487 | 29:36108259 | 111492 | 29:36148048 | 111495 | 29:36206784 | 111496 | 29:36207683 |
| 111505 | 29:36388797 | 111511 | 29:36481048 | 111520 | 29:36766544 | 111521 | 29:36775708 | 111522 | 29:36800395 |
| 111523 | 29:36811950 | 111524 | 29:36813596 | 111530 | 29:36934958 | 111533 | 29:36975121 | 111545 | 29:37168472 |
| 111548 | 29:37221849 | 111553 | 29:37332886 | 111554 | 29:37332893 | 111555 | 29:37342276 | 111558 | 29:37411101 |
| 111561 | 29:37440660 | 111562 | 29:37458594 | 111564 | 29:37523548 | 111600 | 29:39536492 | 111617 | 29:39891282 |
| 111622 | 29:39975025 | 111630 | 29:40151594 | 111633 | 29:40297458 | 111634 | 29:40314843 | 111643 | 29:40481400 |
| 111645 | 29:40518208 | 111648 | 29:40595649 | 111656 | 29:40805372 | 111657 | 29:40806225 | 111668 | 29:40851566 |
| 111670 | 29:40878959 | 111671 | 29:40879481 | 111674 | 29:40919327 | 111679 | 29:41004847 | 111681 | 29:41038278 |
| 111684 | 29:41142315 | 111685 | 29:41142793 | 111693 | 29:41279012 | 111697 | 29:41338346 | 111698 | 29:41342454 |
| 111706 | 29:41493354 | 111710 | 29:41557548 | 111711 | 29:41564691 | 111714 | 29:41642963 | 111716 | 29:41687163 |
| 111718 | 29:41749646 | 111721 | 29:41785049 | 111722 | 29:41789280 | 111723 | 29:41792341 | 111725 | 29:41823721 |
| 111729 | 29:41947165 | 111731 | 29:41976753 | 111737 | 29:42018005 | 111738 | 29:42022273 | 111739 | 29:42031453 |
| 111753 | 29:42391692 | 111758 | 29:42509147 | 111759 | 29:42513501 | 111761 | 29:42528204 | 111762 | 29:42615699 |
| 111764 | 29:42635388 | 111765 | 29:42642354 | 111767 | 29:42657038 | 111768 | 29:42659255 | 111769 | 29:42659448 |
| 111777 | 29:42844516 | 111781 | 29:42982094 | 111785 | 29:43052218 | 111787 | 29:43090186 | 111790 | 29:43206957 |
| 111791 | 29:43207132 | 111792 | 29:43207525 | 111793 | 29:43207573 | 111794 | 29:43207753 | 111795 | 29:43210991 |
| 111796 | 29:43218240 | 111801 | 29:43282578 | 111805 | 29:43391770 | 111812 | 29:43495617 | 111815 | 29:43524321 |
| 111823 | 29:43620118 | 111824 | 29:43622532 | 111825 | 29:43627877 | 111826 | 29:43631768 | 111830 | 29:43704875 |
| 111832 | 29:43750816 | 111833 | 29:43750820 | 111834 | 29:43814690 | 111835 | 29:43827364 | 111837 | 29:43880340 |
| 111840 | 29:43883371 | 111842 | 29:43909067 | 111845 | 29:43968492 | 111846 | 29:43970000 | 111848 | 29:43981948 |
| 111849 | 29:43982210 | 111865 | 29:44265806 | 111866 | 29:44285086 | 111868 | 29:44314568 | 111877 | 29:44423551 |
| 111881 | 29:44484826 | 111884 | 29:44552650 | 111888 | 29:44627679 | 111894 | 29:44770084 | 111899 | 29:44991893 |
| 111906 | 29:45224759 | 111911 | 29:45378882 | 111914 | 29:45460407 | 111918 | 29:45538531 | 111920 | 29:45566486 |
| 111921 | 29:45574701 | 111922 | 29:45601782 | 111929 | 29:45749221 | 111930 | 29:45756539 | 111934 | 29:45875877 |
| 111935 | 29:45897665 | 111939 | 29:45951010 | 111940 | 29:45996529 | 111944 | 29:46069944 | 111953 | 29:46268948 |
| 111958 | 29:46344185 | 111960 | 29:46344338 | 111990 | 29:47058184 | 111993 | 29:47089599 | 111997 | 29:47171895 |
| 112007 | 29:47412530 | 112009 | 29:47512321 | 112016 | 29:47628422 | 112032 | 29:47965964 | 112035 | 29:48004279 |
| 112041 | 29:48072601 | 112050 | 29:48380729 | 112051 | 29:48412845 | 112053 | 29:48499387 | 112064 | 29:48750418 |
| 112072 | 29:48886024 | 112073 | 29:48936775 | 112102 | 29:49277470 | 112111 | 29:49503212 | 112113 | 29:49583754 |
| 112114 | 29:49601539 | 112117 | 29:49645960 | 112118 | 29:49646022 | 112123 | 29:49706299 | 112136 | 29:50015783 |
| 112142 | 29:50279949 | 112143 | 29:50279990 | 112145 | 29:50326170 | 112152 | 29:50499209 | 112156 | 29:50566734 |
| 112157 | 29:50572585 | 112158 | 29:50573514 | 112159 | 29:50595441 | 112165 | 29:50698048 | 112166 | 29:50698051 |
| 112167 | 29:50698125 | 112168 | 29:50699876 | 112169 | 29:50700624 | 112170 | 29:50702620 | 112172 | 29:50777521 |
| 112173 | 29:50809985 | 112176 | 29:50901915 | 112177 | 29:50902670 | | | | |

In the present disclosure, a third marker panel was preferably a total of 249 SNPs for identifying genetic relationship, and the SNPs information was shown in Table 3:

TABLE 3
Location information of third marker panel

| NO. | Position | NO. | Position | NO. | Position | NO. | Position | NO. | Position |
|---|---|---|---|---|---|---|---|---|---|
| 188 | 1:3958867 | 339 | 1:7037229 | 420 | 1:9069741 | 1171 | 1:29963400 | 1536 | 1:40576723 |
| 2248 | 1:58965351 | 2403 | 1:62673903 | 3852 | 1:98519700 | 4392 | 1:113176039 | 4969 | 1:126428831 |
| 6050 | 1:149646917 | 6468 | 1:156749195 | 6762 | 2:5374531 | 6787 | 2:5824469 | 6980 | 2:10425003 |
| 7049 | 2:12802862 | 7090 | 2:13822009 | 7681 | 2:26949128 | 8255 | 2:41823284 | 8310 | 2:43164529 |
| 8401 | 2:45730559 | 10904 | 2:110396989 | 11196 | 2:117962757 | 11661 | 2:128269460 | 12778 | 3:12671675 |
| 13137 | 3:21144762 | 14032 | 3:39549189 | 14060 | 3:40255504 | 14105 | 3:41605228 | 14424 | 3:49548031 |
| 14538 | 3:51817697 | 14750 | 3:57896539 | 15299 | 3:73432513 | 15739 | 3:84990774 | 16150 | 3:93914273 |
| 16285 | 3:97595840 | 17239 | 3:115822760 | 17989 | 4:10906324 | 18181 | 4:16215151 | 18389 | 4:20652215 |
| 18975 | 4:36742038 | 19158 | 4:41400156 | 19719 | 4:57421426 | 20196 | 4:70610326 | 20258 | 4:72532921 |
| 20479 | 4:77917876 | 21974 | 4:109990416 | 22886 | 5:7656578 | 23714 | 5:27670995 | 23888 | 5:31691766 |
| 25051 | 5:62933839 | 25204 | 5:66693946 | 25436 | 5:71914501 | 25686 | 5:78046443 | 26221 | 5:87923946 |
| 26711 | 5:97631203 | 27506 | 5:112616318 | 27834 | 5:118152314 | 28511 | 6:12732175 | 28954 | 6:22210848 |
| 29193 | 6:28113107 | 29194 | 6:28131847 | 32264 | 6:42994429 | 32505 | 6:45364629 | 33094 | 6:59551603 |
| 33110 | 6:59868907 | 33550 | 6:72852551 | 35075 | 6:109291738 | 35278 | 6:113484974 | 35919 | 7:7248738 |
| 36059 | 7:12174901 | 36345 | 7:17223080 | 36713 | 7:24861430 | 37909 | 7:53233377 | 39036 | 7:79304309 |
| 39041 | 7:79381437 | 39108 | 7:80536102 | 39619 | 7:91858328 | 39707 | 7:94529183 | 40120 | 7:100955064 |
| 40310 | 7:105797741 | 40557 | 8:1688535 | 41676 | 8:28747847 | 42844 | 8:59585336 | 43949 | 8:87523043 |
| 44493 | 8:101431379 | 44662 | 8:104418638 | 45531 | 9:14224642 | 46080 | 9:27778476 | 47046 | 9:51702071 |
| 47294 | 9:58511465 | 48324 | 9:82208636 | 49082 | 9:97029776 | 49666 | 10:3582438 | 50211 | 10:14601498 |
| 50331 | 10:16803986 | 51431 | 10:44059494 | 51467 | 10:44872711 | 51923 | 10:55539558 | 52128 | 10:61462408 |
| 52940 | 10:81223782 | 53009 | 10:82399475 | 53079 | 10:84068155 | 53672 | 10:97152695 | 54484 | 11:9400474 |
| 55098 | 11:24487930 | 55582 | 11:36861491 | 55963 | 11:46544231 | 56603 | 11:62042659 | 56631 | 11:62781436 |
| 56787 | 11:66365240 | 57355 | 11:77817486 | 58187 | 11:95543179 | 58516 | 11:102567442 | 58559 | 11:103007477 |
| 59180 | 12:11793754 | 59773 | 12:25646113 | 59990 | 12:31268165 | 60909 | 12:57867430 | 60996 | 12:60997744 |
| 61249 | 12:68116940 | 61552 | 12:75672295 | 61568 | 12:76081859 | 62170 | 13:2079650 | 63173 | 13:25335505 |
| 64229 | 13:47037532 | 65139 | 13:68527135 | 65425 | 13:74664505 | 65845 | 13:82667365 | 66385 | 14:9116686 |
| 66433 | 14:10036916 | 67576 | 14:26068764 | 68401 | 14:46226278 | 68669 | 14:53479348 | 68965 | 14:60298957 |
| 69693 | 14:77723052 | 69777 | 14:79615272 | 70897 | 15:20899423 | 71481 | 15:32912616 | 71704 | 15:37472272 |
| 71706 | 15:37547916 | 71942 | 15:42887040 | 72290 | 15:49801112 | 72317 | 15:50693401 | 72976 | 15:63833091 |
| 73595 | 15:77950839 | 74258 | 16:9259631 | 74330 | 16:11622161 | 74400 | 16:13072437 | 74604 | 16:19197451 |
| 76750 | 16:71035799 | 76969 | 16:75329647 | 77277 | 17:1023299 | 77660 | 17:9132112 | 77811 | 17:12726620 |
| 78014 | 17:17314187 | 78466 | 17:29524233 | 78477 | 17:29949936 | 78539 | 17:31478059 | 79296 | 17:50485602 |
| 79468 | 17:54264980 | 79988 | 17:64965089 | 80500 | 18:1801945 | 80747 | 18:6747828 | 81503 | 18:23351423 |
| 81570 | 18:24699063 | 81666 | 18:26757520 | 82513 | 18:46459862 | 82680 | 18:48545897 | 82834 | 18:52178290 |
| 83457 | 18:64214636 | 83717 | 19:4478391 | 83895 | 19:8274910 | 84037 | 19:10861227 | 84235 | 19:15017885 |
| 84328 | 19:16900665 | 85539 | 19:38864468 | 85580 | 19:40043192 | 85799 | 19:44173520 | 86248 | 19:52293262 |
| 86366 | 19:54554240 | 86736 | 19:59907369 | 87086 | 20:2386360 | 87954 | 20:17847390 | 88540 | 20:30911469 |
| 89177 | 20:46043226 | 89624 | 20:58379906 | 89839 | 20:63586403 | 90346 | 21:3040671 | 91142 | 21:20809473 |
| 91348 | 21:26120612 | 91396 | 21:27418010 | 91923 | 21:39994586 | 92052 | 21:43133914 | 92411 | 21:51692379 |
| 92884 | 21:60444453 | 93013 | 21:63550319 | 93835 | 22:11000418 | 93860 | 22:11718954 | 94244 | 22:20916356 |
| 94281 | 22:21678074 | 94310 | 22:22468414 | 94409 | 22:25488681 | 95460 | 22:50589283 | 95591 | 22:53477312 |
| 95731 | 22:55883157 | 96358 | 23:7318640 | 97405 | 23:27495679 | 98150 | 23:40993623 | 98205 | 23:42021817 |
| 98687 | 23:51029219 | 98839 | 24:1617199 | 99035 | 24:6125588 | 99289 | 24:10497132 | 99465 | 24:15141986 |
| 99707 | 24:21378400 | 100652 | 24:44195277 | 101189 | 24:55950028 | 101747 | 25:3130565 | 102307 | 25:14593246 |
| 102533 | 25:19902652 | 102870 | 25:27084643 | 102896 | 25:27654242 | 104001 | 26:8192400 | 104205 | 26:13194710 |
| 105241 | 26:37900334 | 105317 | 26:39609490 | 105761 | 26:49826945 | 106248 | 27:9204910 | 106327 | 27:11736207 |
| 106418 | 27:13894404 | 106551 | 27:16084911 | 106705 | 27:19194136 | 106831 | 27:22410377 | 107482 | 27:35761644 |
| 107588 | 27:37824983 | 107723 | 27:41177956 | 108116 | 28:5874287 | 108524 | 28:15972452 | 108715 | 28:20371221 |
| 109327 | 28:33248298 | 109425 | 28:35132199 | 109901 | 28:43879624 | 110370 | 29:9104654 | 110458 | 29:11198143 |
| 111111 | 29:28278699 | 111128 | 29:28684366 | 111855 | 29:44090714 | 112174 | 29:50858136 | | |

In the present disclosure, a fourth marker panel was preferably integrated with the relevant research results of the inventor's team in the previous period and published literature in PUBMED. The specific traits included 73 traits in five categories including growth, fattening, carcass, meat quality, and genetic diseases of beef cattle. Applied methods included single-trait GWAS, multi-trait GWAS, wssGWAS, Bayesian analysis, selection signal detection, haplotype, CNV and ROH analysis. A total of 3,909 markers were obtained, including 3,907 functional SNPs and 2 Indels. The information of fourth marker panel was shown in Table 4:

TABLE 4
Location information of fourth marker panel

| NO. | Position | NO | Position | NO. | Position | NO. | Position | NO. | Position |
|---|---|---|---|---|---|---|---|---|---|
| 41 | 1:1299095 | 42 | 1:1301411 | 43 | 1:1302242 | 72 | 1:1675530 | 92 | 1:2080108 |
| 105 | 1:2256758 | 163 | 1:3595729 | 167 | 1:3620513 | 211 | 1:4298693 | 229 | 1:4729108 |
| 234 | 1:4821451 | 287 | 1:6049970 | 288 | 1:6052470 | 289 | 1:6054551 | 290 | 1:6056604 |
| 291 | 1:6061246 | 373 | 1:7795006 | 388 | 1:8249174 | 413 | 1:8847132 | 417 | 1:8982763 |
| 421 | 1:9088695 | 423 | 1:9118340 | 508 | 1:10838284 | 509 | 1:10876705 | 536 | 1:11604750 |
| 538 | 1:11752900 | 546 | 1:12052406 | 553 | 1:12362501 | 567 | 1:12653822 | 629 | 1:14114440 |
| 630 | 1:14176114 | 721 | 1:16635130 | 728 | 1:16922627 | 747 | 1:17462003 | 756 | 1:17835385 |
| 763 | 1:18110024 | 785 | 1:18793633 | 806 | 1:19543445 | 813 | 1:19664427 | 930 | 1:22893609 |
| 948 | 1:23486726 | 949 | 1:23493262 | 969 | 1:24006741 | 984 | 1:24500834 | 996 | 1:24941722 |
| 1001 | 1:25068708 | 1025 | 1:25577980 | 1129 | 1:29025677 | 1131 | 1:29040742 | 1225 | 1:31664947 |
| 1237 | 1:32296642 | 1256 | 1:32911987 | 1277 | 1:33660199 | 1286 | 1:33837966 | 1287 | 1:33843390 |
| 1288 | 1:33853447 | 1447 | 1:37890316 | 1452 | 1:38007855 | 1516 | 1:39993737 | 1545 | 1:40753557 |
| 1577 | 1:41764542 | 1622 | 1:42618603 | 1693 | 1:44281769 | 1695 | 1:44303304 | 1702 | 1:44457020 |
| 1705 | 1:44558674 | 1834 | 1:47759114 | 1879 | 1:49422581 | 1896 | 1:49969737 | 2024 | 1:53619617 |
| 2025 | 1:53623176 | 2026 | 1:53624697 | 2027 | 1:53629301 | 2028 | 1:53630113 | 2050 | 1:54016006 |
| 2075 | 1:54444238 | 2084 | 1:54690665 | 2116 | 1:55714510 | 2139 | 1:56372440 | 2335 | 1:61145631 |
| 2343 | 1:61319735 | 2362 | 1:61796684 | 2383 | 1:62427513 | 2384 | 1:62433558 | 2430 | 1:63221728 |
| 2462 | 1:64193131 | 2467 | 1:64262857 | 2472 | 1:64316563 | 2499 | 1:64907342 | 2526 | 1:65509479 |
| 2543 | 1:66070553 | 2602 | 1:67691264 | 2624 | 1:68339701 | 2675 | 1:69057373 | 2701 | 1:69645645 |
| 2739 | 1:70443699 | 2766 | 1:70688109 | 2800 | 1:71512292 | 2916 | 1:74460838 | 2934 | 1:74972802 |
| 2936 | 1:74999820 | 2946 | 1:75290882 | 2981 | 1:76075161 | 3007 | 1:76874012 | 3018 | 1:77236634 |
| 3022 | 1:77276581 | 3039 | 1:77623057 | 3041 | 1:77668859 | 3043 | 1:77697633 | 3046 | 1:77710839 |
| 3047 | 1:77723763 | 3060 | 1:78064459 | 3068 | 1:78315800 | 3079 | 1:78505641 | 3100 | 1:79138567 |
| 3115 | 1:79395243 | 3125 | 1:79597067 | 3165 | 1:80126772 | 3190 | 1:80929167 | 3251 | 1:82240750 |
| 3261 | 1:82501216 | 3275 | 1:82703570 | 3311 | 1:83985563 | 3319 | 1:84218035 | 3324 | 1:84478357 |
| 3325 | 1:84493668 | 3336 | 1:85023039 | 3352 | 1:85456698 | 3354 | 1:85501954 | 3382 | 1:86229426 |
| 3407 | 1:86980692 | 3460 | 1:88794416 | 3464 | 1:88824366 | 3545 | 1:90506530 | 3610 | 1:92190821 |
| 3612 | 1:92200875 | 3624 | 1:92563287 | 3638 | 1:93096031 | 3652 | 1:93351663 | 3654 | 1:93372952 |
| 3698 | 1:94573894 | 3724 | 1:95244942 | 3763 | 1:96215196 | 3768 | 1:96305965 | 3790 | 1:96826926 |
| 3791 | 1:96855345 | 3796 | 1:96968145 | 3806 | 1:97319365 | 3829 | 1:97847508 | 3860 | 1:98721777 |
| 3881 | 1:99293036 | 3929 | 1:100616517 | 3939 | 1:100987282 | 4172 | 1:108128123 | 4199 | 1:108777437 |
| 4225 | 1:109419276 | 4227 | 1:109445956 | 4261 | 1:110075319 | 4281 | 1:110389266 | 4305 | 1:111001754 |
| 4330 | 1:111772744 | 4338 | 1:111960165 | 4374 | 1:112577975 | 4386 | 1:112928362 | 4408 | 1:113414092 |
| 4421 | 1:113728857 | 4466 | 1:114828912 | 4467 | 1:114837165 | 4479 | 1:115162087 | 4480 | 1:115169020 |
| 4482 | 1:115183751 | 4521 | 1:116624187 | 4523 | 1:116668988 | 4524 | 1:116683980 | 4567 | 1:117562616 |
| 4604 | 1:118172592 | 4606 | 1:118199028 | 4607 | 1:118200955 | 4660 | 1:119155291 | 4714 | 1:120407128 |
| 4767 | 1:121845822 | 4796 | 1:122567824 | 4957 | 1:126246695 | 4964 | 1:126332119 | 5053 | 1:128692434 |
| 5062 | 1:128825902 | 5078 | 1:129263274 | 5234 | 1:132164555 | 5244 | 1:132550146 | 5263 | 1:133371805 |
| 5292 | 1:134238188 | 5295 | 1:134324146 | 5308 | 1:134489385 | 5335 | 1:135158239 | 5350 | 1:135504827 |
| 5358 | 1:135602604 | 5392 | 1:135798830 | 5450 | 1:137640097 | 5470 | 1:138610452 | 5474 | 1:138652323 |
| 5496 | 1:139117138 | 5556 | 1:140791857 | 5596 | 1:141481667 | 5678 | 1:142851690 | 5679 | 1:142863118 |
| 5697 | 1:143175205 | 5769 | 1:144589518 | 5777 | 1:144761021 | 5823 | 1:145661981 | 5912 | 1:147299699 |
| 5924 | 1:147514403 | 5943 | 1:147887268 | 5993 | 1:148725405 | 6105 | 1:150672403 | 6192 | 1:152095555 |
| 6198 | 1:152196690 | 6272 | 1:153106216 | 6277 | 1:153197360 | 6309 | 1:153828547 | 6340 | 1:154314444 |
| 6357 | 1:154855892 | 6360 | 1:154863975 | 6511 | 1:157806548 | 6568 | 2:1106317 | 6575 | 2:1169877 |
| 6578 | 2:1187683 | 6617 | 2:1860169 | 6621 | 2:1971521 | 6640 | 2:2590948 | 6666 | 2:3510323 |
| 6673 | 2:3709938 | 6718 | 2:4623152 | 6790 | 2:5906646 | 6800 | 2:6055644 | 6971 | 2:10245713 |
| 6979 | 2:10421191 | 7005 | 2:11387282 | 7060 | 2:13168802 | 7064 | 2:13243953 | 7176 | 2:16006718 |
| 7184 | 2:16189620 | 7214 | 2:16976649 | 7240 | 2:17639779 | 7274 | 2:18268568 | 7332 | 2:19056221 |
| 7365 | 2:19833213 | 7582 | 2:24849703 | 7630 | 2:25885564 | 7655 | 2:26463668 | 7685 | 2:27015449 |
| 7704 | 2:27459813 | 7705 | 2:27463924 | 7708 | 2:27525591 | 7760 | 2:28545094 | 7777 | 2:28922483 |
| 7807 | 2:29732106 | 7811 | 2:29813146 | 7889 | 2:31637856 | 7906 | 2:32169593 | 8046 | 2:36375875 |
| 8097 | 2:38039323 | 8098 | 2:38039898 | 8099 | 2:38041017 | 8100 | 2:38042315 | 8116 | 2:38297286 |
| 8120 | 2:38435225 | 8134 | 2:38750534 | 8153 | 2:39280801 | 8169 | 2:39667440 | 8177 | 2:39982069 |
| 8197 | 2:40615263 | 8225 | 2:41307524 | 8262 | 2:42027595 | 8270 | 2:42236387 | 8280 | 2:42439089 |
| 8281 | 2:42440990 | 8298 | 2:42711951 | 8329 | 2:43789161 | 8389 | 2:45494226 | 8399 | 2:45665663 |
| 8419 | 2:46116917 | 8473 | 2:47217427 | 8516 | 2:48495171 | 8517 | 2:48499243 | 8518 | 2:48502118 |
| 8519 | 2:48508248 | 8520 | 2:48511801 | 8560 | 2:49760042 | 8561 | 2:49760735 | 8611 | 2:51218510 |
| 8704 | 2:53518329 | 8730 | 2:54413562 | 8742 | 2:54672099 | 8762 | 2:55031934 | 8764 | 2:55069900 |
| 8765 | 2:55071242 | 8876 | 2:58381419 | 8884 | 2:58588299 | 8913 | 2:59316972 | 8960 | 2:60576956 |
| 8969 | 2:60779135 | 9031 | 2:62457322 | 9119 | 2:64653571 | 9135 | 2:64967881 | 9147 | 2:65185518 |
| 9201 | 2:66960608 | 9235 | 2:68024571 | 9384 | 2:72399848 | 9389 | 2:72514078 | 9418 | 2:73276155 |
| 9479 | 2:74974797 | 9480 | 2:74985065 | 9502 | 2:75739779 | 9535 | 2:76454912 | 9626 | 2:78834490 |
| 9641 | 2:79036468 | 9657 | 2:79510060 | 9681 | 2:80059990 | 9698 | 2:80586285 | 9747 | 2:83003800 |
| 9751 | 2:83109314 | 9755 | 2:83267596 | 9786 | 2:83877880 | 9795 | 2:84259043 | 9799 | 2:84375486 |
| 9832 | 2:85066770 | 9833 | 2:85070174 | 9851 | 2:85646050 | 9887 | 2:86425660 | 9898 | 2:86617347 |
| 9914 | 2:87174608 | 9921 | 2:87410708 | 9941 | 2:88035286 | 9972 | 2:88739478 | 10037 | 2:89511031 |
| 10038 | 2:89515068 | 10039 | 2:89518986 | 10040 | 2:89521529 | 10041 | 2:89523615 | 10076 | 2:90510385 |
| 10078 | 2:90533958 | 10114 | 2:91721645 | 10127 | 2:92066999 | 10128 | 2:92069312 | 10129 | 2:92085813 |
| 10130 | 2:92089545 | 10160 | 2:92756276 | 10181 | 2:93326093 | 10199 | 2:93469951 | 10200 | 2:93470548 |
| 10201 | 2:93471304 | 10202 | 2:93472560 | 10203 | 2:93473816 | 10218 | 2:93915068 | 10222 | 2:94006514 |
| 10223 | 2:94010942 | 10280 | 2:95335798 | 10338 | 2:96515244 | 10370 | 2:97455980 | 10377 | 2:97722574 |
| 10387 | 2:97882323 | 10401 | 2:98337087 | 10410 | 2:98432307 | 10413 | 2:98461459 | 10468 | 2:99951594 |
| 10492 | 2:100538824 | 10513 | 2:101147607 | 10555 | 2:102376439 | 10596 | 2:103531207 | 10650 | 2:104919346 |
| 10658 | 2:105027822 | 10663 | 2:105088054 | 10675 | 2:105412051 | 10829 | 2:108708703 | 10863 | 2:109439523 |
| 11027 | 2:113672892 | 11061 | 2:114526247 | 11202 | 2:118124674 | 11224 | 2:118587782 | 11225 | 2:118669657 |
| 11279 | 2:119878226 | 11336 | 2:121220519 | 11355 | 2:121820038 | 11356 | 2:121865885 | 11406 | 2:123297173 |
| 11455 | 2:124641406 | 11484 | 2:125479428 | 11617 | 2:127271059 | 11649 | 2:127896093 | 11743 | 2:130052119 |
| 11744 | 2:130058714 | 11745 | 2:130070890 | 11859 | 2:132478188 | 11860 | 2:132523448 | 11870 | 2:132734429 |
| 11917 | 2:133633752 | 11918 | 2:133637575 | 11939 | 2:133848234 | 11953 | 2:134015104 | 12115 | 3:525997 |
| 12118 | 3:556754 | 12143 | 3:950659 | 12145 | 3:954886 | 12153 | 3:1098608 | 12197 | 3:1960686 |
| 12261 | 3:3372085 | 12263 | 3:3434999 | 12280 | 3:3763587 | 12294 | 3:4101542 | 12315 | 3:4378889 |
| 12321 | 3:4437644 | 12343 | 3:4727841 | 12348 | 3:4795643 | 12369 | 3:5161089 | 12400 | 3:5776030 |
| 12410 | 3:6048242 | 12493 | 3:7480890 | 12527 | 3:7942262 | 12558 | 3:8433782 | 12561 | 3:8460244 |
| 12563 | 3:8520080 | 12564 | 3:8526638 | 12565 | 3:8529009 | 12566 | 3:8532372 | 12617 | 3:10020876 |
| 12620 | 3:10090524 | 12736 | 3:11477492 | 12754 | 3:11784186 | 12882 | 3:14796654 | 12934 | 3:15837312 |
| 13018 | 3:17753548 | 13048 | 3:18771436 | 13049 | 3:18775018 | 13050 | 3:18791656 | 13051 | 3:18795346 |
| 13056 | 3:18930543 | 13237 | 3:23451211 | 13314 | 3:25255575 | 13325 | 3:25405368 | 13343 | 3:25977712 |
| 13383 | 3:26543166 | 13431 | 3:27586116 | 13490 | 3:28659529 | 13578 | 3:30031426 | 13589 | 3:30407397 |
| 13632 | 3:31383515 | 13728 | 3:32928510 | 13731 | 3:32940407 | 13891 | 3:35285151 | 13909 | 3:35786483 |
| 13913 | 3:35908304 | 13922 | 3:36096832 | 13927 | 3:36253878 | 13990 | 3:37885414 | 14009 | 3:38624906 |
| 14010 | 3:38630748 | 14044 | 3:39845695 | 14058 | 3:40207827 | 14122 | 3:42050876 | 14139 | 3:42539907 |
| 14163 | 3:42995977 | 14232 | 3:44866434 | 14327 | 3:47649002 | 14398 | 3:49141481 | 14485 | 3:50111050 |
| 14521 | 3:51439192 | 14529 | 3:51625928 | 14618 | 3:53775526 | 14654 | 3:55087957 | 14667 | 3:55359809 |
| 14686 | 3:56006266 | 14688 | 3:56067950 | 14793 | 3:59053277 | 14797 | 3:59211837 | 14806 | 3:59443494 |
| 14818 | 3:59847747 | 14845 | 3:60575455 | 14847 | 3:60611151 | 14982 | 3:64188398 | 15009 | 3:65159985 |
| 15035 | 3:65721928 | 15088 | 3:66944634 | 15124 | 3:67769302 | 15169 | 3:68874408 | 15180 | 3:69234087 |
| 15187 | 3:69431230 | 15225 | 3:70347328 | 15264 | 3:72041156 | 15338 | 3:74664014 | 15421 | 3:77158063 |
| 15495 | 3:79128870 | 15545 | 3:80676731 | 15627 | 3:82625118 | 15652 | 3:83549724 | 15659 | 3:83658003 |
| 15683 | 3:84227201 | 15750 | 3:85215781 | 15768 | 3:85766170 | 15778 | 3:85960283 | 15779 | 3:85964163 |
| 15780 | 3:85967104 | 15781 | 3:85968892 | 15782 | 3:85969726 | 15798 | 3:86300913 | 15807 | 3:86441083 |
| 15908 | 3:88661891 | 15916 | 3:88782949 | 15917 | 3:88784034 | 15935 | 3:89220299 | 15977 | 3:89929116 |
| 16037 | 3:91288695 | 16170 | 3:94375264 | 16194 | 3:95152254 | 16204 | 3:95487322 | 16235 | 3:96416999 |
| 16276 | 3:97475924 | 16328 | 3:98728026 | 16330 | 3:98766843 | 16366 | 3:99717720 | 16400 | 3:100301019 |
| 16439 | 3:100971847 | 16445 | 3:101130049 | 16459 | 3:101339407 | 16485 | 3:101926220 | 16537 | 3:102883747 |
| 16572 | 3:103741473 | 16602 | 3:104484207 | 16605 | 3:104506752 | 16620 | 3:104710861 | 16639 | 3:105175424 |
| 16646 | 3:105209686 | 16684 | 3:105603312 | 16714 | 3:105965567 | 16715 | 3:105973059 | 16740 | 3:106492826 |
| 16774 | 3:107138615 | 16811 | 3:107842370 | 16874 | 3:109092493 | 16881 | 3:109399680 | 16887 | 3:109428951 |
| 16913 | 3:109808282 | 16959 | 3:110964139 | 17043 | 3:112687905 | 17134 | 3:114337662 | 17162 | 3:114723123 |
| 17166 | 3:114787475 | 17167 | 3:114787630 | 17168 | 3:114788049 | 17169 | 3:114788632 | 17170 | 3:114789131 |
| 17172 | 3:114791529 | 17173 | 3:114799492 | 17176 | 3:114803442 | 17177 | 3:114803451 | 17178 | 3:114803465 |
| 17179 | 3:114804161 | 17180 | 3:114804367 | 17195 | 3:115056511 | 17196 | 3:115071383 | 17206 | 3:115299586 |
| 17211 | 3:115381373 | 17407 | 3:118290616 | 17448 | 3:118879237 | 17676 | 4:4683962 | 17696 | 4:5003827 |
| 17732 | 4:5560351 | 17733 | 4:5568155 | 17760 | 4:6327312 | 17789 | 4:7010182 | 17804 | 4:7324916 |
| 17806 | 4:7326126 | 17855 | 4:8442378 | 17985 | 4:10842025 | 18045 | 4:12514592 | 18143 | 4:15186933 |
| 18155 | 4:15475039 | 18161 | 4:15588873 | 18162 | 4:15592728 | 18180 | 4:16210509 | 18257 | 4:18112247 |
| 18263 | 4:18273472 | 18273 | 4:18595000 | 18299 | 4:18985118 | 18400 | 4:20821405 | 18441 | 4:21738699 |
| 18444 | 4:21789346 | 18448 | 4:21942101 | 18464 | 4:22381610 | 18492 | 4:22930826 | 18520 | 4:23861032 |
| 18617 | 4:26517471 | 18748 | 4:30365149 | 18770 | 4:30805170 | 18813 | 4:31905587 | 18817 | 4:31972387 |
| 18879 | 4:33771936 | 18945 | 4:35882843 | 19025 | 4:38124757 | 19039 | 4:38555013 | 19086 | 4:40002914 |
| 19087 | 4:40007566 | 19088 | 4:40012097 | 19089 | 4:40018785 | 19090 | 4:40021130 | 19091 | 4:40041810 |
| 19092 | 4:40049842 | 19093 | 4:40054177 | 19094 | 4:40059057 | 19095 | 4:40060306 | 19115 | 4:40634211 |
| 19116 | 4:40640519 | 19117 | 4:40645360 | 19118 | 4:40649968 | 19119 | 4:40654713 | 19167 | 4:41814723 |
| 19172 | 4:42014141 | 19183 | 4:42356867 | 19190 | 4:42554917 | 19255 | 4:44244202 | 19291 | 4:45415922 |
| 19296 | 4:45536174 | 19308 | 4:45825175 | 19335 | 4:46718903 | 19342 | 4:46848225 | 19357 | 4:47216690 |
| 19358 | 4:47217662 | 19359 | 4:47221067 | 19360 | 4:47224473 | 19361 | 4:47228083 | 19400 | 4:48100380 |
| 19463 | 4:49408307 | 19575 | 4:53093575 | 19600 | 4:53722558 | 19607 | 4:53807295 | 19720 | 4:57434269 |
| 19763 | 4:58913134 | 19783 | 4:59391253 | 19787 | 4:59489126 | 19813 | 4:60378132 | 19818 | 4:60482892 |
| 19853 | 4:61414628 | 19854 | 4:61423853 | 19855 | 4:61432230 | 19856 | 4:61434936 | 19857 | 4:61439997 |
| 19881 | 4:62128232 | 19885 | 4:62171821 | 19916 | 4:63134475 | 19951 | 4:63940606 | 19960 | 4:64270176 |
| 19961 | 4:64271209 | 19964 | 4:64495002 | 19995 | 4:65572773 | 20043 | 4:66416290 | 20063 | 4:67092276 |
| 20082 | 4:67523624 | 20114 | 4:68300881 | 20190 | 4:70507936 | 20193 | 4:70569760 | 20246 | 4:72066682 |
| 20297 | 4:73725730 | 20302 | 4:73904776 | 20343 | 4:74843205 | 20361 | 4:75190744 | 20382 | 4:75792193 |
| 20390 | 4:75908626 | 20442 | 4:77149758 | 20462 | 4:77552330 | 20482 | 4:77954957 | 20489 | 4:78153455 |
| 20519 | 4:79093225 | 20523 | 4:79236024 | 20527 | 4:79350296 | 20537 | 4:79637506 | 20607 | 4:81119261 |
| 20687 | 4:83090213 | 20854 | 4:87615934 | 20860 | 4:87832774 | 20862 | 4:87887042 | 20866 | 4:87931932 |
| 20867 | 4:87937433 | 20918 | 4:89596848 | 20927 | 4:89756186 | 20933 | 4:89854090 | 20952 | 4:90405270 |
| 20965 | 4:90752463 | 20983 | 4:91186390 | 21013 | 4:91662043 | 21020 | 4:91835951 | 21026 | 4:91985689 |
| 21064 | 4:92683225 | 21143 | 4:94586866 | 21162 | 4:95121580 | 21208 | 4:95976133 | 21209 | 4:95978012 |
| 21210 | 4:95984102 | 21211 | 4:95986472 | 21212 | 4:95988223 | 21213 | 4:95991905 | 21214 | 4:95994577 |
| 21215 | 4:95996626 | 21216 | 4:95999064 | 21217 | 4:95999599 | 21218 | 4:95999657 | 21220 | 4:96006244 |
| 21223 | 4:96012342 | 21224 | 4:96014135 | 21225 | 4:96014452 | 21226 | 4:96014495 | 21229 | 4:96043384 |
| 21230 | 4:96043881 | 21258 | 4:96806394 | 21259 | 4:96808296 | 21260 | 4:96808305 | 21261 | 4:96808793 |
| 21263 | 4:96810164 | 21264 | 4:96810688 | 21265 | 4:96810739 | 21266 | 4:96811491 | 21267 | 4:96811492 |
| 21268 | 4:96811663 | 21269 | 4:96819713 | 21270 | 4:96819807 | 21271 | 4:96819821 | 21272 | 4:96820983 |
| 21273 | 4:96821494 | 21274 | 4:96822544 | 21275 | 4:96822709 | 21276 | 4:96822722 | 21277 | 4:96823395 |
| 21278 | 4:96823570 | 21280 | 4:96826910 | 21281 | 4:96827344 | 21282 | 4:96828046 | 21283 | 4:96828753 |
| 21284 | 4:96829524 | 21286 | 4:96842438 | 21287 | 4:96842474 | 21288 | 4:96846828 | 21289 | 4:96847175 |
| 21290 | 4:96847270 | 21291 | 4:96847298 | 21292 | 4:96847522 | 21293 | 4:96847685 | 21294 | 4:96848163 |
| 21295 | 4:96852063 | 21296 | 4:96852112 | 21297 | 4:96852128 | 21299 | 4:96853114 | 21300 | 4:96853149 |
| 21301 | 4:96854041 | 21302 | 4:96854052 | 21303 | 4:96854217 | 21304 | 4:96854309 | 21305 | 4:96854674 |
| 21306 | 4:96854753 | 21307 | 4:96854840 | 21308 | 4:96854846 | 21309 | 4:96855073 | 21310 | 4:96855142 |
| 21311 | 4:96855211 | 21312 | 4:96855234 | 21313 | 4:96855382 | 21314 | 4:96858356 | 21315 | 4:96858411 |
| 21316 | 4:96858620 | 21317 | 4:96859305 | 21318 | 4:96859677 | 21319 | 4:96859857 | 21320 | 4:96860289 |
| 21321 | 4:96860821 | 21322 | 4:96861792 | 21323 | 4:96862653 | 21324 | 4:96868903 | 21325 | 4:96870132 |
| 21326 | 4:96870621 | 21327 | 4:96871036 | 21328 | 4:96871329 | 21329 | 4:96872714 | 21330 | 4:96872734 |
| 21331 | 4:96878187 | 21332 | 4:96878242 | 21333 | 4:96878278 | 21334 | 4:96880890 | 21335 | 4:96880962 |
| 21336 | 4:96881026 | 21337 | 4:96881161 | 21338 | 4:96881966 | 21339 | 4:96882244 | 21340 | 4:96885170 |
| 21341 | 4:96886853 | 21342 | 4:96887160 | 21343 | 4:96887442 | 21344 | 4:96887486 | 21345 | 4:96887810 |
| 21346 | 4:96888595 | 21347 | 4:96889148 | 21348 | 4:96890359 | 21349 | 4:96890450 | 21350 | 4:96890701 |
| 21351 | 4:96891069 | 21352 | 4:96891335 | 21353 | 4:96891542 | 21354 | 4:96891552 | 21355 | 4:96891608 |
| 21356 | 4:96895620 | 21357 | 4:96897050 | 21358 | 4:96897145 | 21367 | 4:97039076 | 21370 | 4:97088268 |
| 21372 | 4:97137855 | 21373 | 4:97147752 | 21374 | 4:97148005 | 21375 | 4:97148878 | 21376 | 4:97150124 |
| 21377 | 4:97150300 | 21378 | 4:97151421 | 21379 | 4:97151944 | 21380 | 4:97152001 | 21381 | 4:97152026 |
| 21382 | 4:97152080 | 21383 | 4:97152151 | 21384 | 4:97152177 | 21385 | 4:97152300 | 21386 | 4:97152327 |
| 21387 | 4:97152343 | 21388 | 4:97152464 | 21389 | 4:97152562 | 21390 | 4:97152880 | 21391 | 4:97153058 |
| 21392 | 4:97160379 | 21393 | 4:97160479 | 21394 | 4:97160507 | 21395 | 4:97160725 | 21396 | 4:97160764 |
| 21397 | 4:97160849 | 21398 | 4:97160967 | 21399 | 4:97161387 | 21400 | 4:97161554 | 21401 | 4:97161881 |
| 21402 | 4:97161958 | 21404 | 4:97164436 | 21405 | 4:97165222 | 21406 | 4:97165446 | 21407 | 4:97165671 |
| 21408 | 4:97165867 | 21409 | 4:97167273 | 21410 | 4:97170350 | 21411 | 4:97170722 | 21412 | 4:97170758 |
| 21413 | 4:97170786 | 21415 | 4:97171793 | 21417 | 4:97173637 | 21418 | 4:97173882 | 21420 | 4:97243175 |
| 21429 | 4:97478233 | 21449 | 4:97997496 | 21460 | 4:98301073 | 21488 | 4:99227319 | 21496 | 4:99551602 |
| 21525 | 4:100096858 | 21571 | 4:101147322 | 21621 | 4:102291227 | 21665 | 4:103373158 | 21709 | 4:104494825 |
| 21712 | 4:104541226 | 21740 | 4:105243840 | 21826 | 4:106811124 | 21828 | 4:106840758 | 21903 | 4:108136633 |
| 21927 | 4:108959151 | 21976 | 4:110025220 | 22029 | 4:111137956 | 22031 | 4:111174901 | 22032 | 4:111185441 |
| 22052 | 4:111594642 | 22057 | 4:111656507 | 22081 | 4:112008507 | 22182 | 4:113533731 | 22254 | 4:114468144 |
| 22334 | 4:115504063 | 22339 | 4:115674188 | 22348 | 4:115903138 | 22484 | 4:118639815 | 22639 | 5:1916328 |
| 22664 | 5:2361885 | 22679 | 5:2678940 | 22680 | 5:2710438 | 22682 | 5:2775410 | 22683 | 5:2789169 |
| 22692 | 5:3084344 | 22721 | 5:4106293 | 22747 | 5:4642231 | 22801 | 5:5757045 | 22812 | 5:5962601 |
| 22864 | 5:7150201 | 22875 | 5:7320087 | 22901 | 5:8063807 | 22907 | 5:8295061 | 22914 | 5:8547280 |
| 23001 | 5:10417113 | 23005 | 5:10483003 | 23116 | 5:12929697 | 23147 | 5:13814938 | 23154 | 5:14118702 |
| 23200 | 5:15181434 | 23209 | 5:15382347 | 23335 | 5:18879061 | 23345 | 5:19180052 | 23365 | 5:19703644 |
| 23420 | 5:21141526 | 23424 | 5:21247771 | 23441 | 5:21652414 | 23488 | 5:22524662 | 23553 | 5:23856635 |
| 23577 | 5:24327813 | 23580 | 5:24363604 | 23609 | 5:25025028 | 23638 | 5:25910383 | 23667 | 5:26553269 |
| 23765 | 5:28658495 | 23768 | 5:28689345 | 23795 | 5:29481439 | 23827 | 5:30068971 | 23860 | 5:30880544 |
| 23885 | 5:31649328 | 23903 | 5:32253508 | 23967 | 5:33731249 | 23969 | 5:33743644 | 23990 | 5:34527357 |
| 24023 | 5:35215343 | 24041 | 5:35917350 | 24064 | 5:36532219 | 24164 | 5:39219011 | 24213 | 5:40323694 |
| 24252 | 5:41581614 | 24255 | 5:41737754 | 24277 | 5:42488185 | 24295 | 5:42883310 | 24309 | 5:43239107 |
| 24321 | 5:43556568 | 24332 | 5:43826757 | 24373 | 5:44749275 | 24394 | 5:45422250 | 24412 | 5:45892148 |
| 24469 | 5:47071100 | 24474 | 5:47204283 | 24476 | 5:47210190 | 24491 | 5:47544371 | 24515 | 5:48255910 |
| 24533 | 5:48697507 | 24700 | 5:53392371 | 24725 | 5:54050541 | 24767 | 5:54925075 | 24868 | 5:57315947 |
| 24869 | 5:57316513 | 24870 | 5:57318993 | 24931 | 5:59393649 | 25012 | 5:61592328 | 25049 | 5:62915639 |
| 25065 | 5:63217741 | 25070 | 5:63272705 | 25094 | 5:63826831 | 25189 | 5:66342506 | 25206 | 5:66729461 |
| 25248 | 5:67796979 | 25274 | 5:68252711 | 25308 | 5:69044504 | 25336 | 5:69658212 | 25386 | 5:70977990 |
| 25396 | 5:71157250 | 25405 | 5:71284786 | 25414 | 5:71441252 | 25415 | 5:71451984 | 25452 | 5:72240212 |
| 25455 | 5:72267018 | 25501 | 5:73577352 | 25505 | 5:73676579 | 25512 | 5:73764801 | 25516 | 5:73813825 |
| 25528 | 5:74101395 | 25540 | 5:74482047 | 25592 | 5:75448318 | 25633 | 5:76668839 | 25639 | 5:76766433 |
| 25644 | 5:76890153 | 25683 | 5:77996585 | 25684 | 5:78020028 | 25716 | 5:78821705 | 25736 | 5:79345129 |
| 25744 | 5:79644695 | 25768 | 5:80587434 | 25796 | 5:81407773 | 25800 | 5:81437991 | 25801 | 5:81441963 |
| 25851 | 5:82570119 | 25855 | 5:82593018 | 25856 | 5:82593228 | 25857 | 5:82593982 | 25858 | 5:82594019 |
| 25859 | 5:82594028 | 25860 | 5:82594375 | 25861 | 5:82594380 | 25862 | 5:82595065 | 25863 | 5:82595148 |
| 25864 | 5:82595334 | 25865 | 5:82627761 | 25905 | 5:83646235 | 26064 | 5:84298547 | 26072 | 5:84433271 |
| 26074 | 5:84527341 | 26089 | 5:84948836 | 26145 | 5:86376332 | 26148 | 5:86455776 | 26196 | 5:87435829 |
| 26293 | 5:88856288 | 26361 | 5:90566853 | 26403 | 5:91501158 | 26409 | 5:91665913 | 26415 | 5:91761326 |
| 26432 | 5:92332808 | 26496 | 5:93178439 | 26526 | 5:93790346 | 26533 | 5:93961735 | 26537 | 5:94088171 |
| 26579 | 5:95091626 | 26625 | 5:96054240 | 26685 | 5:97071869 | 26781 | 5:98982241 | 26878 | 5:100843102 |
| 26891 | 5:101049907 | 26948 | 5:102626302 | 27117 | 5:105882827 | 27183 | 5:106801714 | 27187 | 5:106877986 |
| 27206 | 5:107060379 | 27244 | 5:107745197 | 27305 | 5:108794780 | 27306 | 5:108797633 | 27341 | 5:109446738 |
| 27366 | 5:109926959 | 27428 | 5:111036587 | 27579 | 5:114683035 | 27584 | 5:114814526 | 27611 | 5:115497329 |
| 27613 | 5:115504227 | 27623 | 5:115614570 | 27664 | 5:116449909 | 27776 | 5:117284799 | 27782 | 5:117411822 |
| 27811 | 5:117753977 | 27824 | 5:117977735 | 27839 | 5:118288125 | 27856 | 5:118666844 | 27917 | 5:119642118 |
| 27951 | 6:579203 | 28082 | 6:3248784 | 28098 | 6:3406400 | 28122 | 6:3655995 | 28164 | 6:4606383 |
| 28201 | 6:5673122 | 28210 | 6:5813338 | 28269 | 6:6797112 | 28271 | 6:6808370 | 28384 | 6:9740976 |
| 28388 | 6:9896356 | 28418 | 6:10695206 | 28487 | 6:12244147 | 28494 | 6:12408823 | 28501 | 6:12515175 |
| 28516 | 6:12899581 | 28534 | 6:13157939 | 28535 | 6:13158398 | 28536 | 6:13158702 | 28570 | 6:14149976 |
| 28645 | 6:15766628 | 28649 | 6:15875705 | 28664 | 6:16165237 | 28720 | 6:17350197 | 28729 | 6:17546682 |
| 28775 | 6:18247972 | 28798 | 6:18858161 | 28832 | 6:19517669 | 28855 | 6:20067345 | 28903 | 6:21187469 |
| 28951 | 6:22198236 | 28974 | 6:22627641 | 28985 | 6:22838005 | 28994 | 6:23185445 | 29012 | 6:23551846 |
| 29033 | 6:24079559 | 29091 | 6:25420968 | 29092 | 6:25430886 | 29113 | 6:25987389 | 29124 | 6:26122133 |
| 29169 | 6:27357262 | 29187 | 6:27922767 | 29228 | 6:29044156 | 29256 | 6:29526067 | 29268 | 6:29755371 |
| 29360 | 6:30504972 | 29389 | 6:31372994 | 29391 | 6:31402252 | 29427 | 6:32495711 | 29446 | 6:33106068 |
| 29505 | 6:34316541 | 29908 | 6:34880169 | 29988 | 6:35895370 | 29990 | 6:35897546 | 29991 | 6:35897939 |
| 29992 | 6:35906185 | 29993 | 6:35907046 | 29995 | 6:35909397 | 29998 | 6:35925489 | 30025 | 6:36097722 |
| 30135 | 6:36427342 | 30137 | 6:36430013 | 30140 | 6:36432356 | 30143 | 6:36441375 | 30257 | 6:36563183 |
| 30260 | 6:36568261 | 30264 | 6:36572899 | 30333 | 6:36813124 | 30455 | 6:37026863 | 30456 | 6:37027429 |
| 30457 | 6:37027539 | 30458 | 6:37028623 | 30460 | 6:37030480 | 30461 | 6:37032906 | 30462 | 6:37032995 |
| 30467 | 6:37035186 | 30469 | 6:37038017 | 30471 | 6:37056413 | 30475 | 6:37075910 | 30566 | 6:37095299 |
| 30616 | 6:37107589 | 30626 | 6:37117771 | 30629 | 6:37121529 | 30630 | 6:37123023 | 30632 | 6:37130785 |
| 30633 | 6:37134418 | 30636 | 6:37136794 | 30643 | 6:37142174 | 30649 | 6:37149945 | 30685 | 6:37161408 |
| 30736 | 6:37173048 | 30788 | 6:37184577 | 30791 | 6:37188623 | 30792 | 6:37189736 | 30798 | 6:37206928 |
| 30799 | 6:37210690 | 30802 | 6:37214389 | 30803 | 6:37214736 | 30810 | 6:37219609 | 30813 | 6:37221776 |
| 30818 | 6:37227250 | 30820 | 6:37231623 | 30829 | 6:37235373 | 30830 | 6:37235374 | 30831 | 6:37236226 |
| 30844 | 6:37244478 | 30849 | 6:37247874 | 30851 | 6:37250850 | 30852 | 6:37251132 | 30854 | 6:37252978 |
| 30856 | 6:37256488 | 30857 | 6:37260221 | 30858 | 6:37262825 | 30859 | 6:37262995 | 30864 | 6:37269061 |
| 30865 | 6:37270215 | 30866 | 6:37270930 | 30867 | 6:37271635 | 30870 | 6:37281308 | 30871 | 6:37281335 |
| 30872 | 6:37282356 | 30873 | 6:37284874 | 30879 | 6:37286627 | 30880 | 6:37286722 | 30881 | 6:37287306 |
| 30882 | 6:37287423 | 30883 | 6:37287873 | 30884 | 6:37288219 | 30885 | 6:37288483 | 30886 | 6:37288600 |
| 30887 | 6:37288948 | 30888 | 6:37289071 | 30889 | 6:37289572 | 30890 | 6:37289742 | 30891 | 6:37289905 |
| 30892 | 6:37289990 | 30893 | 6:37290180 | 30894 | 6:37290241 | 30895 | 6:37290415 | 30896 | 6:37291360 |
| 30897 | 6:37291500 | 30898 | 6:37292117 | 30899 | 6:37292269 | 30900 | 6:37292270 | 30901 | 6:37292298 |
| 30902 | 6:37292752 | 30903 | 6:37293476 | 30905 | 6:37294505 | 30913 | 6:37297426 | 30917 | 6:37298725 |
| 30927 | 6:37300810 | 30932 | 6:37301959 | 30936 | 6:37302499 | 30938 | 6:37303255 | 30946 | 6:37304513 |
| 30952 | 6:37305466 | 30956 | 6:37306944 | 30959 | 6:37309266 | 30960 | 6:37310413 | 30972 | 6:37313503 |
| 30977 | 6:37314183 | 30993 | 6:37317695 | 31003 | 6:37321049 | 31004 | 6:37321441 | 31010 | 6:37324718 |
| 31018 | 6:37328528 | 31026 | 6:37329878 | 31030 | 6:37330306 | 31032 | 6:37330477 | 31033 | 6:37330577 |
| 31036 | 6:37331712 | 31046 | 6:37335926 | 31052 | 6:37337744 | 31056 | 6:37340937 | 31058 | 6:37342568 |
| 31066 | 6:37345508 | 31070 | 6:37347130 | 31071 | 6:37349144 | 31075 | 6:37351767 | 31076 | 6:37352145 |
| 31086 | 6:37359765 | 31088 | 6:37360685 | 31089 | 6:37361449 | 31090 | 6:37361578 | 31091 | 6:37362262 |
| 31099 | 6:37369338 | 31100 | 6:37369380 | 31102 | 6:37372334 | 31108 | 6:37376289 | 31111 | 6:37378475 |
| 31113 | 6:37379506 | 31114 | 6:37379507 | 31118 | 6:37380161 | 31120 | 6:37381106 | 31122 | 6:37381782 |
| 31123 | 6:37382003 | 31124 | 6:37382062 | 31127 | 6:37383526 | 31129 | 6:37385973 | 31133 | 6:37391915 |
| 31138 | 6:37393949 | 31139 | 6:37394877 | 31140 | 6:37395313 | 31143 | 6:37396789 | 31144 | 6:37398199 |
| 31145 | 6:37399560 | 31147 | 6:37400741 | 31151 | 6:37403131 | 31152 | 6:37403224 | 31153 | 6:37406239 |
| 31154 | 6:37406485 | 31156 | 6:37407653 | 31157 | 6:37412062 | 31162 | 6:37415367 | 31163 | 6:37416259 |
| 31166 | 6:37417501 | 31167 | 6:37418164 | 31169 | 6:37418447 | 31170 | 6:37418449 | 31172 | 6:37420957 |
| 31174 | 6:37424248 | 31176 | 6:37429071 | 31178 | 6:37429776 | 31179 | 6:37430447 | 31180 | 6:37431101 |
| 31181 | 6:37432458 | 31186 | 6:37435861 | 31188 | 6:37436347 | 31191 | 6:37437538 | 31192 | 6:37438222 |
| 31197 | 6:37438993 | 31199 | 6:37440571 | 31204 | 6:37444900 | 31208 | 6:37449890 | 31212 | 6:37457249 |
| 31216 | 6:37465669 | 31217 | 6:37466194 | 31218 | 6:37473453 | 31219 | 6:37477629 | 31221 | 6:37480277 |
| 31222 | 6:37482054 | 31225 | 6:37491305 | 31229 | 6:37503406 | 31230 | 6:37505093 | 31231 | 6:37508184 |
| 31235 | 6:37513645 | 31239 | 6:37522580 | 31240 | 6:37526812 | 31243 | 6:37534297 | 31246 | 6:37553481 |
| 31248 | 6:37560625 | 31249 | 6:37562383 | 31251 | 6:37566268 | 31252 | 6:37566317 | 31254 | 6:37573307 |
| 31255 | 6:37573615 | 31256 | 6:37578832 | 31262 | 6:37587980 | 31263 | 6:37588492 | 31264 | 6:37591326 |
| 31265 | 6:37591664 | 31268 | 6:37598284 | 31276 | 6:37608504 | 31278 | 6:37614179 | 31280 | 6:37624003 |
| 31281 | 6:37625923 | 31282 | 6:37627776 | 31284 | 6:37636883 | 31285 | 6:37636900 | 31286 | 6:37637484 |
| 31287 | 6:37639651 | 31288 | 6:37639763 | 31289 | 6:37640954 | 31290 | 6:37641448 | 31291 | 6:37641798 |
| 31292 | 6:37642775 | 31293 | 6:37644691 | 31294 | 6:37644915 | 31295 | 6:37645671 | 31296 | 6:37646000 |
| 31297 | 6:37646499 | 31301 | 6:37658848 | 31349 | 6:37772675 | 31353 | 6:37774778 | 31355 | 6:37775624 |
| 31357 | 6:37776401 | 31359 | 6:37776454 | 31360 | 6:37776464 | 31361 | 6:37777029 | 31363 | 6:37777185 |
| 31404 | 6:37817575 | 31438 | 6:37912116 | 31445 | 6:37914472 | 31454 | 6:37917366 | 31455 | 6:37917421 |
| 31465 | 6:37919853 | 31475 | 6:37926073 | 31476 | 6:37926600 | 31477 | 6:37926960 | 31478 | 6:37928156 |
| 31484 | 6:37929786 | 31486 | 6:37930252 | 31487 | 6:37930293 | 31488 | 6:37930672 | 31490 | 6:37930868 |
| 31491 | 6:37930889 | 31492 | 6:37931089 | 31493 | 6:37931216 | 31494 | 6:37931298 | 31495 | 6:37931621 |
| 31496 | 6:37931688 | 31499 | 6:37932170 | 31501 | 6:37932241 | 31503 | 6:37932377 | 31504 | 6:37932412 |
| 31506 | 6:37932563 | 31507 | 6:37932682 | 31508 | 6:37932772 | 31509 | 6:37932778 | 31510 | 6:37932960 |
| 31512 | 6:37933352 | 31514 | 6:37934068 | 31518 | 6:37935108 | 31519 | 6:37935168 | 31520 | 6:37935527 |
| 31521 | 6:37935577 | 31522 | 6:37935748 | 31524 | 6:37936087 | 31525 | 6:37936692 | 31526 | 6:37938294 |
| 31527 | 6:37938374 | 31528 | 6:37938826 | 31531 | 6:37942290 | 31532 | 6:37942944 | 31533 | 6:37943635 |
| 31534 | 6:37943860 | 31535 | 6:37943970 | 31536 | 6:37944478 | 31537 | 6:37944482 | 31538 | 6:37944496 |
| 31539 | 6:37944573 | 31541 | 6:37944681 | 31542 | 6:37944704 | 31544 | 6:37945482 | 31545 | 6:37945820 |
| 31546 | 6:37945849 | 31558 | 6:37959360 | 31561 | 6:37961553 | 31563 | 6:37962887 | 31565 | 6:37963525 |
| 31568 | 6:37966382 | 31585 | 6:37974401 | 31586 | 6:37974407 | 31819 | 6:38395086 | 31822 | 6:38409517 |
| 31826 | 6:38491475 | 31831 | 6:38518347 | 31836 | 6:38540452 | 31837 | 6:38541525 | 31839 | 6:38542048 |
| 31842 | 6:38543400 | 31843 | 6:38543545 | 31844 | 6:38543990 | 31845 | 6:38545112 | 31846 | 6:38545113 |
| 31847 | 6:38546290 | 31848 | 6:38546646 | 31849 | 6:38548060 | 31850 | 6:38548198 | 31851 | 6:38548496 |
| 31852 | 6:38548525 | 31853 | 6:38548659 | 31854 | 6:38549044 | 31855 | 6:38549099 | 31856 | 6:38549206 |
| 31857 | 6:38549477 | 31859 | 6:38550162 | 31860 | 6:38550667 | 31861 | 6:38552085 | 31862 | 6:38552158 |
| 31863 | 6:38552167 | 31864 | 6:38552459 | 31865 | 6:38552550 | 31866 | 6:38552644 | 31867 | 6:38552821 |
| 31868 | 6:38553028 | 31869 | 6:38553181 | 31870 | 6:38553447 | 31871 | 6:38553456 | 31872 | 6:38553565 |
| 31873 | 6:38553842 | 31874 | 6:38553946 | 31875 | 6:38554100 | 31876 | 6:38554163 | 31877 | 6:38554439 |
| 31878 | 6:38554455 | 31879 | 6:38554714 | 31880 | 6:38555555 | 31881 | 6:38556220 | 31882 | 6:38556748 |
| 31883 | 6:38556956 | 31884 | 6:38556978 | 31885 | 6:38557241 | 31886 | 6:38557498 | 31887 | 6:38557633 |
| 31888 | 6:38557694 | 31889 | 6:38557830 | 31890 | 6:38557833 | 31891 | 6:38557888 | 31892 | 6:38557940 |
| 31894 | 6:38558946 | 31895 | 6:38562229 | 31897 | 6:38564166 | 31898 | 6:38565084 | 31902 | 6:38583433 |
| 31904 | 6:38584686 | 31908 | 6:38600235 | 31965 | 6:39250952 | 31969 | 6:39301737 | 31989 | 6:39663383 |
| 31992 | 6:39721697 | 31993 | 6:39727899 | 31994 | 6:39730068 | 31995 | 6:39733610 | 31996 | 6:39737879 |
| 32029 | 6:40129309 | 32034 | 6:40237863 | 32062 | 6:40696320 | 32086 | 6:40735514 | 32119 | 6:40952449 |
| 32207 | 6:42123221 | 32266 | 6:43009159 | 32267 | 6:43017852 | 32294 | 6:43462079 | 32329 | 6:43973189 |
| 32339 | 6:44149286 | 32547 | 6:46220665 | 32588 | 6:47190072 | 32619 | 6:47907662 | 32631 | 6:48281817 |
| 32657 | 6:49188655 | 32670 | 6:49492652 | 32675 | 6:49628351 | 32678 | 6:49692019 | 32757 | 6:51655726 |
| 32824 | 6:52798083 | 32827 | 6:52912323 | 32854 | 6:53474037 | 32911 | 6:55287740 | 32947 | 6:55553127 |
| 32949 | 6:55563964 | 32951 | 6:55573386 | 32968 | 6:56049754 | 32972 | 6:56230949 | 33026 | 6:57612544 |
| 33034 | 6:57940998 | 33036 | 6:57996603 | 33038 | 6:58046666 | 33086 | 6:59279953 | 33101 | 6:59756158 |
| 33144 | 6:60551717 | 33151 | 6:60729399 | 33174 | 6:61339528 | 33199 | 6:61996360 | 33215 | 6:62563366 |
| 33218 | 6:62627183 | 33317 | 6:65798913 | 33363 | 6:67312644 | 33364 | 6:67348632 | 33404 | 6:68600503 |
| 33421 | 6:69030371 | 33437 | 6:69395469 | 33438 | 6:69429025 | 33455 | 6:70069795 | 33480 | 6:70868126 |
| 33492 | 6:71178118 | 33506 | 6:71614584 | 33509 | 6:71658863 | 33575 | 6:73737226 | 33611 | 6:75157043 |
| 33635 | 6:75976163 | 33715 | 6:78625022 | 33873 | 6:83484610 | 33909 | 6:84465355 | 33927 | 6:84898498 |
| 34137 | 6:89248038 | 34177 | 6:90260850 | 34180 | 6:90360832 | 34292 | 6:92070503 | 34356 | 6:93561855 |
| 34416 | 6:94977849 | 34431 | 6:95495492 | 34452 | 6:96090013 | 34453 | 6:96097237 | 34476 | 6:96479912 |
| 34478 | 6:96544331 | 34486 | 6:96686083 | 34491 | 6:96916348 | 34531 | 6:97462721 | 34545 | 6:97701064 |
| 34600 | 6:98712630 | 34635 | 6:99753620 | 34663 | 6:100291469 | 34716 | 6:101484421 | 34717 | 6:101486616 |
| 34720 | 6:101524578 | 34735 | 6:101873336 | 34761 | 6:102268756 | 34817 | 6:103176226 | 34828 | 6:103236460 |
| 34912 | 6:105003956 | 35080 | 6:109427077 | 35101 | 6:109822389 | 35163 | 6:111118988 | 35165 | 6:111196630 |
| 35175 | 6:111382228 | 35229 | 6:112604266 | 35271 | 6:113397576 | 35275 | 6:113470683 | 35406 | 6:115996386 |
| 35448 | 6:116540874 | 35480 | 7:310937 | 35482 | 7:325799 | 35558 | 7:1927986 | 35561 | 7:1995591 |
| 35582 | 7:2406915 | 35845 | 7:6222553 | 35873 | 7:6554748 | 35887 | 7:6745523 | 35900 | 7:6956627 |
| 35928 | 7:7534130 | 35936 | 7:7674523 | 35947 | 7:8035405 | 35989 | 7:10310655 | 35990 | 7:10395846 |
| 36049 | 7:11976493 | 36068 | 7:12333943 | 36069 | 7:12335290 | 36140 | 7:13300617 | 36153 | 7:13562003 |
| 36173 | 7:13942658 | 36277 | 7:15601726 | 36310 | 7:16447387 | 36311 | 7:16487854 | 36362 | 7:17629628 |
| 36364 | 7:17639856 | 36392 | 7:18356451 | 36423 | 7:19107522 | 36440 | 7:19307460 | 36503 | 7:20153088 |
| 36525 | 7:20401996 | 36546 | 7:20912796 | 36597 | 7:22016441 | 36603 | 7:22057135 | 36620 | 7:22504243 |
| 36646 | 7:23221425 | 36663 | 7:23603215 | 36683 | 7:24060066 | 36685 | 7:24072015 | 36705 | 7:24634875 |
| 36768 | 7:26043336 | 36828 | 7:27103526 | 36858 | 7:27828159 | 36879 | 7:28407768 | 36890 | 7:28661620 |
| 36941 | 7:29715806 | 37014 | 7:31333882 | 37042 | 7:31763022 | 37047 | 7:31806849 | 37062 | 7:32255462 |
| 37091 | 7:33083761 | 37108 | 7:33452281 | 37175 | 7:35015402 | 37188 | 7:35370637 | 37211 | 7:35997017 |
| 37230 | 7:36507816 | 37294 | 7:37977197 | 37369 | 7:39696107 | 37371 | 7:39720514 | 37528 | 7:43355224 |
| 37544 | 7:43621545 | 37609 | 7:45530009 | 37635 | 7:46401887 | 37725 | 7:48628543 | 37738 | 7:48808046 |
| 37745 | 7:48945061 | 37752 | 7:49169638 | 37886 | 7:52686851 | 37912 | 7:53315105 | 37957 | 7:54541065 |
| 37960 | 7:54622430 | 37975 | 7:55088638 | 38007 | 7:55900961 | 38015 | 7:56078682 | 38033 | 7:56535413 |
| 38035 | 7:56546344 | 38057 | 7:57273769 | 38076 | 7:57910079 | 38077 | 7:57916334 | 38081 | 7:57993375 |
| 38083 | 7:58043785 | 38153 | 7:59374018 | 38159 | 7:59461244 | 38188 | 7:60116908 | 38196 | 7:60319670 |
| 38224 | 7:61001713 | 38269 | 7:61575267 | 38286 | 7:62059589 | 38299 | 7:62381127 | 38312 | 7:62665355 |
| 38313 | 7:62666818 | 38441 | 7:65409794 | 38449 | 7:65594792 | 38520 | 7:66847524 | 38592 | 7:68772935 |
| 38594 | 7:68885530 | 38657 | 7:70110273 | 38683 | 7:71032462 | 38718 | 7:71794897 | 38728 | 7:71996424 |
| 38742 | 7:72314529 | 38808 | 7:74314108 | 38814 | 7:74538065 | 38843 | 7:75119443 | 38849 | 7:75246858 |
| 38850 | 7:75278782 | 38865 | 7:75680840 | 38908 | 7:76699078 | 38924 | 7:77064173 | 38949 | 7:77643339 |
| 38981 | 7:78247892 | 38986 | 7:78358118 | 39067 | 7:79874457 | 39117 | 7:80683144 | 39127 | 7:80972557 |
| 39147 | 7:81218151 | 39197 | 7:82527838 | 39256 | 7:83861078 | 39276 | 7:84283959 | 39317 | 7:85606256 |
| 39369 | 7:87039982 | 39386 | 7:87585340 | 39438 | 7:89078282 | 39458 | 7:89409840 | 39473 | 7:89883975 |
| 39495 | 7:90326242 | 39589 | 7:91305083 | 39633 | 7:92190820 | 39704 | 7:94482521 | 39741 | 7:95245462 |
| 39764 | 7:95873877 | 39776 | 7:96101255 | 39780 | 7:96126223 | 39967 | 7:96962124 | 39977 | 7:97249811 |
| 39984 | 7:97508045 | 39987 | 7:97537955 | 40007 | 7:98034786 | 40009 | 7:98127942 | 40167 | 7:102360026 |
| 40187 | 7:102838629 | 40190 | 7:102879057 | 40252 | 7:104220122 | 40270 | 7:104812484 | 40345 | 7:106558358 |
| 40381 | 7:107255212 | 40401 | 7:108080872 | 40406 | 7:108274048 | 40425 | 7:108662049 | 40435 | 7:108882531 |
| 40540 | 8:1185446 | 40561 | 8:1731044 | 40562 | 8:1735452 | 40568 | 8:1797826 | 40584 | 8:2112938 |
| 40597 | 8:2381761 | 40609 | 8:2664904 | 40720 | 8:5502050 | 40787 | 8:7227540 | 40820 | 8:7866886 |
| 40852 | 8:8701133 | 40988 | 8:11839675 | 41027 | 8:12939122 | 41083 | 8:14812117 | 41084 | 8:14836734 |
| 41129 | 8:15805194 | 41130 | 8:15826955 | 41140 | 8:16188640 | 41161 | 8:16768290 | 41295 | 8:20088784 |
| 41367 | 8:22206603 | 41372 | 8:22296675 | 41379 | 8:22531070 | 41384 | 8:22832882 | 41391 | 8:23108057 |
| 41574 | 8:25836147 | 41585 | 8:26072917 | 41683 | 8:28986201 | 41684 | 8:29004667 | 41685 | 8:29005955 |
| 41710 | 8:29655591 | 41727 | 8:29969758 | 41806 | 8:32023600 | 41820 | 8:32402575 | 41826 | 8:32658867 |
| 41872 | 8:34205302 | 41887 | 8:34586231 | 41924 | 8:35462325 | 41970 | 8:36379322 | 41971 | 8:36399226 |
| 42048 | 8:38716745 | 42085 | 8:39915480 | 42086 | 8:39942972 | 42119 | 8:40483328 | 42137 | 8:40876799 |
| 42195 | 8:42367753 | 42266 | 8:44367476 | 42313 | 8:45640500 | 42355 | 8:46379679 | 42373 | 8:46931988 |
| 42389 | 8:47348455 | 42415 | 8:47938185 | 42433 | 8:48578484 | 42492 | 8:50146172 | 42546 | 8:51588079 |
| 42631 | 8:53806641 | 42639 | 8:54114529 | 42646 | 8:54256907 | 42654 | 8:54525130 | 42656 | 8:54533690 |
| 42657 | 8:54534313 | 42658 | 8:54536088 | 42659 | 8:54544973 | 42660 | 8:54547153 | 42854 | 8:59769666 |
| 42892 | 8:60472436 | 42903 | 8:60785213 | 43085 | 8:65667278 | 43089 | 8:65893239 | 43110 | 8:66320758 |
| 43186 | 8:67933084 | 43215 | 8:68749952 | 43230 | 8:69106626 | 43232 | 8:69110799 | 43244 | 8:69435453 |
| 43345 | 8:72058951 | 43397 | 8:73337772 | 43434 | 8:74223661 | 43566 | 8:77624055 | 43579 | 8:78130709 |
| 43593 | 8:78494713 | 43655 | 8:80608965 | 43668 | 8:80925878 | 43679 | 8:81135408 | 43745 | 8:82415599 |
| 43764 | 8:82948559 | 43847 | 8:85052405 | 43927 | 8:87126956 | 43963 | 8:87960883 | 43972 | 8:88206973 |
| 43973 | 8:88207717 | 44068 | 8:90411690 | 44089 | 8:90899216 | 44097 | 8:91046537 | 44301 | 8:96913052 |
| 44325 | 8:97476895 | 44365 | 8:98665857 | 44390 | 8:99176725 | 44398 | 8:99311311 | 44414 | 8:99547619 |
| 44418 | 8:99577548 | 44438 | 8:100015023 | 44490 | 8:101376581 | 44635 | 8:103813682 | 44653 | 8:104231786 |
| 44746 | 8:106532357 | 44749 | 8:106594158 | 44792 | 8:107582608 | 45042 | 9:2688546 | 45058 | 9:3203477 |
| 45130 | 9:5470319 | 45151 | 9:6116416 | 45173 | 9:6877480 | 45177 | 9:7031515 | 45229 | 9:8388451 |
| 45283 | 9:9884501 | 45296 | 9:10301651 | 45298 | 9:10310168 | 45417 | 9:11927500 | 45439 | 9:12374207 |
| 45514 | 9:13742514 | 45529 | 9:14218593 | 45532 | 9:14239370 | 45566 | 9:15019469 | 45593 | 9:15465522 |
| 45684 | 9:17366049 | 45686 | 9:17428879 | 45709 | 9:18143920 | 45711 | 9:18153000 | 45712 | 9:18160118 |
| 45757 | 9:19618032 | 45977 | 9:25228881 | 45987 | 9:25512136 | 46000 | 9:25838484 | 46031 | 9:26618713 |
| 46034 | 9:26691579 | 46063 | 9:27473901 | 46065 | 9:27477523 | 46066 | 9:27485623 | 46095 | 9:28146296 |
| 46123 | 9:28679350 | 46135 | 9:28927068 | 46242 | 9:30976439 | 46245 | 9:31003562 | 46276 | 9:31748693 |
| 46330 | 9:33062497 | 46343 | 9:33376755 | 46364 | 9:33886935 | 46372 | 9:34155670 | 46516 | 9:37778578 |
| 46545 | 9:38309457 | 46546 | 9:38314365 | 46561 | 9:38550987 | 46644 | 9:40310425 | 46767 | 9:43367400 |
| 46771 | 9:43430747 | 46802 | 9:44301570 | 46864 | 9:46172575 | 46865 | 9:46201814 | 46916 | 9:47818319 |
| 46943 | 9:48865487 | 46946 | 9:48902577 | 46963 | 9:49476706 | 46974 | 9:49653803 | 47048 | 9:51753467 |
| 47067 | 9:52274853 | 47097 | 9:52875308 | 47148 | 9:54342081 | 47149 | 9:54361703 | 47168 | 9:54807613 |
| 47325 | 9:59403628 | 47340 | 9:59637965 | 47341 | 9:59638253 | 47342 | 9:59641031 | 47343 | 9:59643481 |
| 47344 | 9:59645062 | 47345 | 9:59645804 | 47346 | 9:59647105 | 47347 | 9:59650376 | 47349 | 9:59653721 |
| 47385 | 9:60709648 | 47395 | 9:60924554 | 47396 | 9:60926410 | 47425 | 9:61795735 | 47611 | 9:65961632 |
| 47667 | 9:67443259 | 47669 | 9:67511922 | 47679 | 9:67708264 | 47688 | 9:68029230 | 47727 | 9:69025344 |
| 47770 | 9:70119558 | 47782 | 9:70360272 | 47843 | 9:71633299 | 47985 | 9:75491653 | 48031 | 9:76631634 |
| 48186 | 9:79336612 | 48219 | 9:80142294 | 48257 | 9:80938431 | 48267 | 9:81108364 | 48274 | 9:81217242 |
| 48305 | 9:81845883 | 48312 | 9:82017648 | 48340 | 9:82626166 | 48342 | 9:82647502 | 48343 | 9:82651491 |
| 48345 | 9:82668654 | 48348 | 9:82682681 | 48354 | 9:82701768 | 48361 | 9:82842810 | 48394 | 9:83693006 |
| 48397 | 9:83826419 | 48468 | 9:85456175 | 48490 | 9:85784712 | 48550 | 9:87315819 | 48553 | 9:87327271 |
| 48558 | 9:87361936 | 48559 | 9:87373306 | 48590 | 9:87819557 | 48591 | 9:87822671 | 48627 | 9:88634584 |
| 48631 | 9:88656403 | 48632 | 9:88680123 | 48707 | 9:89908123 | 48708 | 9:89918140 | 48710 | 9:89921487 |
| 48713 | 9:90012380 | 48748 | 9:90826907 | 48780 | 9:91531504 | 48810 | 9:92215180 | 48815 | 9:92295269 |
| 48856 | 9:93221725 | 48880 | 9:93749307 | 48891 | 9:93889254 | 48911 | 9:94252780 | 48914 | 9:94269583 |
| 48923 | 9:94459307 | 48924 | 9:94493145 | 48986 | 9:95568256 | 49048 | 9:96604431 | 49052 | 9:96651231 |
| 49053 | 9:96652474 | 49054 | 9:96655491 | 49055 | 9:96658405 | 49056 | 9:96659811 | 49059 | 9:96727179 |
| 49060 | 9:96730432 | 49065 | 9:96818068 | 49100 | 9:97292214 | 49186 | 9:99056298 | 49199 | 9:99304134 |
| 49204 | 9:99370466 | 49267 | 9:100494397 | 49269 | 9:100511637 | 49277 | 9:100658454 | 49280 | 9:100721863 |
| 49342 | 9:101817009 | 49343 | 9:101818874 | 49344 | 9:101822513 | 49346 | 9:101827562 | 49349 | 9:101865213 |
| 49442 | 9:103263715 | 49469 | 9:103800979 | 49529 | 10:872754 | 49531 | 10:882204 | 49542 | 10:1178970 |
| 49569 | 10:1795897 | 49614 | 10:2626851 | 49654 | 10:3355041 | 49730 | 10:4924755 | 49746 | 10:5330263 |
| 49804 | 10:6194603 | 49811 | 10:6389416 | 49855 | 10:7506260 | 49856 | 10:7508517 | 49896 | 10:8296974 |
| 49909 | 10:8505165 | 49939 | 10:9303914 | 49984 | 10:10211405 | 49995 | 10:10425877 | 50019 | 10:10791977 |
| 50022 | 10:10887987 | 50067 | 10:11918946 | 50097 | 10:12461852 | 50162 | 10:13597734 | 50172 | 10:13748622 |
| 50235 | 10:15125993 | 50326 | 10:16719617 | 50347 | 10:17070966 | 50355 | 10:17235899 | 50385 | 10:17893631 |
| 50510 | 10:20312528 | 50512 | 10:20320283 | 50597 | 10:21831782 | 50627 | 10:22355891 | 50730 | 10:26724399 |
| 50740 | 10:26816725 | 50748 | 10:26936267 | 50763 | 10:27119455 | 50820 | 10:28726853 | 50827 | 10:28818762 |
| 50840 | 10:28923770 | 50844 | 10:28933854 | 50884 | 10:29514735 | 50885 | 10:29522253 | 50886 | 10:29533360 |
| 50906 | 10:30075494 | 50909 | 10:30126897 | 50911 | 10:30167171 | 50955 | 10:31703939 | 50968 | 10:32063661 |
| 50983 | 10:32536622 | 51010 | 10:33047373 | 51015 | 10:33221086 | 51073 | 10:34533777 | 51093 | 10:34950052 |
| 51125 | 10:35727830 | 51189 | 10:37345559 | 51190 | 10:37377718 | 51191 | 10:37388003 | 51202 | 10:37683947 |
| 51210 | 10:37775786 | 51223 | 10:38166114 | 51227 | 10:38269447 | 51243 | 10:38653361 | 51265 | 10:39263333 |
| 51295 | 10:40078678 | 51332 | 10:41115727 | 51348 | 10:41693430 | 51354 | 10:41758465 | 51445 | 10:44433737 |
| 51454 | 10:44645299 | 51494 | 10:45580212 | 51611 | 10:47920359 | 51626 | 10:48320530 | 51643 | 10:48547551 |
| 51688 | 10:49516649 | 51721 | 10:50223805 | 51725 | 10:50334170 | 51759 | 10:51169084 | 51760 | 10:51181102 |
| 51761 | 10:51186753 | 51773 | 10:51492328 | 51784 | 10:51754878 | 51809 | 10:52286224 | 51840 | 10:52879725 |
| 51889 | 10:54463903 | 51952 | 10:56290718 | 51963 | 10:56443542 | 52006 | 10:57713193 | 52037 | 10:58360318 |
| 52049 | 10:58658013 | 52120 | 10:61117665 | 52181 | 10:62385028 | 52245 | 10:64094738 | 52272 | 10:64614710 |
| 52276 | 10:64656067 | 52345 | 10:66304541 | 52390 | 10:67276588 | 52391 | 10:67310241 | 52399 | 10:67673108 |
| 52467 | 10:68971489 | 52480 | 10:69334027 | 52486 | 10:69501262 | 52499 | 10:69823517 | 52501 | 10:69836682 |
| 52504 | 10:69941547 | 52513 | 10:70197389 | 52537 | 10:71023784 | 52552 | 10:71449748 | 52570 | 10:72023102 |
| 52598 | 10:73101962 | 52623 | 10:73660537 | 52653 | 10:74459889 | 52665 | 10:75024558 | 52672 | 10:75197354 |
| 52688 | 10:75607741 | 52743 | 10:76813636 | 52823 | 10:78537516 | 52826 | 10:78594348 | 52840 | 10:79035728 |
| 52900 | 10:80684424 | 52913 | 10:81068664 | 52970 | 10:81760396 | 53006 | 10:82276845 | 53023 | 10:82877025 |
| 53054 | 10:83522156 | 53055 | 10:83550757 | 53062 | 10:83712797 | 53068 | 10:83857093 | 53071 | 10:83936676 |
| 53099 | 10:84417728 | 53239 | 10:87507691 | 53287 | 10:88767077 | 53305 | 10:89008507 | 53312 | 10:89144342 |
| 53340 | 10:89729222 | 53366 | 10:90207696 | 53388 | 10:90615371 | 53487 | 10:92661034 | 53497 | 10:92892557 |
| 53545 | 10:94160502 | 53555 | 10:94348103 | 53604 | 10:95590054 | 53623 | 10:95920911 | 53627 | 10:96023548 |
| 53669 | 10:97104354 | 53675 | 10:97169425 | 53745 | 10:98522222 | 53797 | 10:99634106 | 53869 | 10:101120062 |
| 53875 | 10:101177760 | 53882 | 10:101281677 | 53887 | 10:101426015 | 53913 | 10:101819543 | 53944 | 10:102268731 |
| 54003 | 11:441012 | 54045 | 11:1204347 | 54069 | 11:1549638 | 54111 | 11:1989902 | 54174 | 11:2689033 |
| 54209 | 11:3235907 | 54259 | 11:4054643 | 54292 | 11:4984752 | 54300 | 11:5106453 | 54327 | 11:5677169 |
| 54409 | 11:7670104 | 54425 | 11:8021628 | 54476 | 11:9219377 | 54572 | 11:11342389 | 54630 | 11:12715868 |
| 54638 | 11:12870807 | 54674 | 11:13642677 | 54677 | 11:13704625 | 54736 | 11:15343003 | 54745 | 11:15626371 |
| 54747 | 11:15658858 | 54756 | 11:15853957 | 54797 | 11:16775998 | 54856 | 11:18554101 | 54872 | 11:18844419 |
| 54875 | 11:18948217 | 54883 | 11:19173839 | 54923 | 11:20562842 | 54924 | 11:20568845 | 54981 | 11:21832414 |
| 55037 | 11:23157077 | 55048 | 11:23494343 | 55084 | 11:24320520 | 55092 | 11:24369984 | 55093 | 11:24377983 |
| 55094 | 11:24393545 | 55096 | 11:24408850 | 55119 | 11:24944857 | 55151 | 11:25930515 | 55161 | 11:26154577 |
| 55174 | 11:26362493 | 55214 | 11:27414983 | 55238 | 11:27959917 | 55283 | 11:28844947 | 55302 | 11:29230653 |
| 55315 | 11:29502309 | 55352 | 11:30379793 | 55380 | 11:30989937 | 55381 | 11:31003265 | 55382 | 11:31020736 |
| 55383 | 11:31023833 | 55425 | 11:32229026 | 55458 | 11:33116089 | 55479 | 11:33643610 | 55496 | 11:34099362 |
| 55532 | 11:35548594 | 55538 | 11:35660110 | 55568 | 11:36429662 | 55588 | 11:37003872 | 55608 | 11:37326478 |
| 55615 | 11:37508364 | 55620 | 11:37570757 | 55624 | 11:37639983 | 55768 | 11:41573665 | 55824 | 11:42850632 |
| 55835 | 11:43167995 | 55873 | 11:44279092 | 55880 | 11:44503647 | 55915 | 11:45256103 | 55925 | 11:45835142 |
| 55933 | 11:45970733 | 55938 | 11:46039317 | 55941 | 11:46045608 | 55942 | 11:46058932 | 55972 | 11:46675342 |
| 56017 | 11:47326731 | 56043 | 11:47992234 | 56067 | 11:48522658 | 56126 | 11:49051072 | 56190 | 11:50710309 |
| 56222 | 11:51848453 | 56258 | 11:53253320 | 56273 | 11:53826835 | 56332 | 11:55052140 | 56335 | 11:55137862 |
| 56337 | 11:55149593 | 56397 | 11:56859473 | 56653 | 11:63292715 | 56682 | 11:63832921 | 56683 | 11:63837249 |
| 56714 | 11:64511310 | 56734 | 11:65119714 | 56739 | 11:65250246 | 56752 | 11:65585594 | 56764 | 11:65826676 |
| 56767 | 11:66038610 | 56784 | 11:66319597 | 56812 | 11:67082495 | 56896 | 11:68461614 | 56913 | 11:68937779 |
| 56917 | 11:69060122 | 56924 | 11:69109200 | 56925 | 11:69118745 | 56928 | 11:69142947 | 56933 | 11:69181613 |
| 56943 | 11:69335989 | 57008 | 11:70675473 | 57082 | 11:71401956 | 57156 | 11:73126654 | 57200 | 11:74123227 |
| 57271 | 11:75902686 | 57350 | 11:77659975 | 57451 | 11:79225059 | 57506 | 11:80496876 | 57543 | 11:81586331 |
| 57554 | 11:81811191 | 57559 | 11:81888932 | 57573 | 11:82234600 | 57724 | 11:85632013 | 57806 | 11:87517972 |
| 57868 | 11:88067171 | 57900 | 11:88940240 | 57915 | 11:89270730 | 57969 | 11:90490496 | 57973 | 11:90559289 |
| 57995 | 11:91106611 | 58020 | 11:91612293 | 58045 | 11:92156116 | 58120 | 11:93813789 | 58133 | 11:94097451 |
| 58153 | 11:94563151 | 58180 | 11:95385268 | 58195 | 11:95727670 | 58219 | 11:96378020 | 58238 | 11:97095593 |
| 58246 | 11:97197864 | 58308 | 11:98538984 | 58364 | 11:99473375 | 58417 | 11:100532361 | 58536 | 11:102829142 |
| 58591 | 11:103534827 | 58607 | 11:104097856 | 58720 | 11:106485352 | 58877 | 12:3502498 | 58943 | 12:5186588 |
| 58945 | 12:5325638 | 58966 | 12:6004879 | 58987 | 12:6773018 | 59008 | 12:7507940 | 59043 | 12:8830007 |
| 59057 | 12:9272154 | 59061 | 12:9329563 | 59069 | 12:9522013 | 59119 | 12:10577179 | 59120 | 12:10585260 |
| 59155 | 12:11315928 | 59176 | 12:11720838 | 59179 | 12:11790945 | 59182 | 12:11827023 | 59184 | 12:11857024 |
| 59208 | 12:12366690 | 59238 | 12:13384011 | 59260 | 12:13823314 | 59276 | 12:14077064 | 59282 | 12:14150866 |
| 59301 | 12:14486563 | 59316 | 12:14918565 | 59397 | 12:16476525 | 59417 | 12:16974977 | 59428 | 12:17253883 |
| 59434 | 12:17359694 | 59439 | 12:17465924 | 59454 | 12:17933719 | 59471 | 12:18284739 | 59549 | 12:20109323 |
| 59698 | 12:23940231 | 59702 | 12:24068026 | 59727 | 12:24577728 | 59786 | 12:25839029 | 59799 | 12:26457768 |
| 59840 | 12:27377754 | 59846 | 12:27488955 | 59870 | 12:28122480 | 59956 | 12:30649835 | 59989 | 12:31264310 |
| 60008 | 12:31780482 | 60117 | 12:34750935 | 60139 | 12:35299411 | 60141 | 12:35311925 | 60142 | 12:35312992 |
| 60143 | 12:35314580 | 60144 | 12:35321842 | 60145 | 12:35326856 | 60147 | 12:35334692 | 60148 | 12:35339381 |
| 60149 | 12:35342141 | 60151 | 12:35355118 | 60152 | 12:35355785 | 60153 | 12:35363362 | 60154 | 12:35424126 |
| 60156 | 12:35487159 | 60202 | 12:37059151 | 60206 | 12:37121439 | 60347 | 12:42536086 | 60352 | 12:42838412 |
| 60389 | 12:44505965 | 60458 | 12:46806766 | 60555 | 12:49288668 | 60578 | 12:49872127 | 60583 | 12:49903248 |
| 60596 | 12:50116234 | 60604 | 12:50253408 | 60730 | 12:53098596 | 60840 | 12:55588645 | 60862 | 12:56409050 |
| 60875 | 12:56736797 | 60935 | 12:58879036 | 61043 | 12:62502730 | 61081 | 12:63602083 | 61084 | 12:63713299 |
| 61163 | 12:65908229 | 61179 | 12:66475638 | 61325 | 12:69535302 | 61424 | 12:73231474 | 61483 | 12:74215241 |
| 61488 | 12:74344277 | 61565 | 12:76057266 | 61574 | 12:76294965 | 61575 | 12:76298488 | 61597 | 12:76881665 |
| 61771 | 12:80709446 | 61777 | 12:80884100 | 61814 | 12:81734916 | 61831 | 12:82087879 | 61844 | 12:82441644 |
| 61856 | 12:82591128 | 61912 | 12:83854133 | 61961 | 12:84933491 | 61993 | 12:85357065 | 62129 | 13:1206621 |
| 62198 | 13:2453852 | 62215 | 13:2711565 | 62223 | 13:2835285 | 62289 | 13:4735864 | 62347 | 13:5936143 |
| 62362 | 13:6285905 | 62525 | 13:10419094 | 62677 | 13:14935831 | 62696 | 13:15681048 | 62771 | 13:17258775 |
| 62815 | 13:17832527 | 62916 | 13:19454627 | 62947 | 13:19907032 | 62968 | 13:20390279 | 62969 | 13:20405413 |
| 62970 | 13:20411494 | 62971 | 13:20423875 | 62972 | 13:20430234 | 62988 | 13:20701775 | 63002 | 13:21060977 |
| 63055 | 13:22024146 | 63112 | 13:23989482 | 63141 | 13:24644133 | 63143 | 13:24667549 | 63150 | 13:24857890 |
| 63174 | 13:25338856 | 63181 | 13:25455632 | 63193 | 13:25724492 | 63199 | 13:25901617 | 63240 | 13:27082022 |
| 63282 | 13:27909495 | 63285 | 13:28007570 | 63295 | 13:28335861 | 63305 | 13:28545136 | 63341 | 13:29355083 |
| 63359 | 13:29815935 | 63424 | 13:31076841 | 63427 | 13:31114025 | 63436 | 13:31276115 | 63470 | 13:31684931 |
| 63484 | 13:32188070 | 63485 | 13:32192701 | 63486 | 13:32195543 | 63488 | 13:32202239 | 63490 | 13:32214896 |
| 63507 | 13:32509667 | 63513 | 13:32595507 | 63515 | 13:32606184 | 63519 | 13:32649822 | 63520 | 13:32651100 |
| 63644 | 13:35586171 | 63673 | 13:36380278 | 63730 | 13:37696035 | 63799 | 13:39119033 | 63842 | 13:39974325 |
| 63843 | 13:40001169 | 63848 | 13:40028952 | 63884 | 13:40680902 | 63923 | 13:41808005 | 63934 | 13:41965173 |
| 63957 | 13:42527459 | 64015 | 13:43212135 | 64028 | 13:43604201 | 64041 | 13:43854540 | 64042 | 13:43855779 |
| 64043 | 13:43868655 | 64044 | 13:43869987 | 64045 | 13:43870636 | 64046 | 13:43872533 | 64047 | 13:43873040 |
| 64048 | 13:43873873 | 64049 | 13:43876772 | 64050 | 13:43878363 | 64051 | 13:43879388 | 64052 | 13:43880125 |
| 64053 | 13:43884214 | 64258 | 13:47680398 | 64280 | 13:48210593 | 64321 | 13:49204706 | 64383 | 13:51177851 |
| 64397 | 13:51341948 | 64439 | 13:52703815 | 64510 | 13:53977365 | 64538 | 13:54845724 | 64978 | 13:64716430 |
| 64982 | 13:64796939 | 64998 | 13:65331493 | 65047 | 13:66193660 | 65061 | 13:66407606 | 65093 | 13:67181101 |
| 65096 | 13:67321578 | 65215 | 13:70526426 | 65221 | 13:70575324 | 65237 | 13:70871738 | 65271 | 13:71637090 |
| 65328 | 13:72957542 | 65357 | 13:73471150 | 65381 | 13:73812183 | 65456 | 13:75531070 | 65529 | 13:76798256 |
| 65547 | 13:77244637 | 65550 | 13:77279091 | 65578 | 13:77845959 | 65598 | 13:78300577 | 65660 | 13:79330804 |
| 65677 | 13:79542295 | 65819 | 13:82171173 | 65917 | 14:1204169 | 65929 | 14:1645654 | 66049 | 14:3651551 |
| 66072 | 14:4085451 | 66078 | 14:4200331 | 66116 | 14:4961976 | 66160 | 14:5700495 | 66209 | 14:6316411 |
| 66268 | 14:7449642 | 66271 | 14:7527096 | 66317 | 14:8213069 | 66475 | 14:10912061 | 66523 | 14:11744884 |
| 66538 | 14:12080094 | 66544 | 14:12195646 | 66565 | 14:12541653 | 66566 | 14:12551654 | 66568 | 14:12570995 |
| 66627 | 14:14043986 | 66718 | 14:16010978 | 66719 | 14:16022809 | 66722 | 14:16058309 | 66732 | 14:16280608 |
| 66746 | 14:16568761 | 66820 | 14:18106996 | 66821 | 14:18115961 | 66855 | 14:19074429 | 66858 | 14:19172311 |
| 66877 | 14:19623114 | 66903 | 14:20293557 | 66912 | 14:20629249 | 66916 | 14:20662609 | 66917 | 14:20673221 |
| 66925 | 14:20792640 | 67108 | 14:22916046 | 67140 | 14:22986080 | 67146 | 14:22999212 | 67302 | 14:23338890 |
| 67351 | 14:23565143 | 67354 | 14:23600267 | 67355 | 14:23607944 | 67357 | 14:23630896 | 67359 | 14:23652804 |
| 67369 | 14:23826318 | 67371 | 14:23829066 | 67374 | 14:23831133 | 67501 | 14:24941221 | 67613 | 14:26283998 |
| 67660 | 14:27607287 | 67681 | 14:28330433 | 67709 | 14:29096850 | 67747 | 14:29873926 | 67772 | 14:30389540 |
| 67822 | 14:31570898 | 67845 | 14:32267064 | 67901 | 14:33278402 | 67955 | 14:34370911 | 68032 | 14:36313786 |
| 68053 | 14:36889990 | 68099 | 14:38260754 | 68128 | 14:39143238 | 68172 | 14:40259820 | 68190 | 14:40976286 |
| 68197 | 14:41229576 | 68198 | 14:41237524 | 68261 | 14:42746359 | 68276 | 14:43241727 | 68313 | 14:44566510 |
| 68321 | 14:44666155 | 68354 | 14:45237526 | 68360 | 14:45367206 | 68364 | 14:45505076 | 68368 | 14:45610432 |
| 68441 | 14:46995812 | 68448 | 14:47163738 | 68460 | 14:47428677 | 68478 | 14:48150783 | 68509 | 14:48767270 |
| 68536 | 14:49487643 | 68580 | 14:50970280 | 68581 | 14:50986924 | 68606 | 14:51659332 | 68620 | 14:51985037 |
| 68654 | 14:52815945 | 68668 | 14:53442111 | 68687 | 14:54022606 | 68730 | 14:54568169 | 68772 | 14:55536222 |
| 68826 | 14:57247647 | 68861 | 14:57991139 | 68870 | 14:58182404 | 68871 | 14:58201975 | 68907 | 14:58964090 |
| 68974 | 14:60591022 | 68975 | 14:60610629 | 68988 | 14:60975742 | 69085 | 14:62782594 | 69115 | 14:63450732 |
| 69130 | 14:63928126 | 69158 | 14:64538337 | 69184 | 14:65420223 | 69193 | 14:65688670 | 69200 | 14:65782382 |
| 69231 | 14:66405235 | 69239 | 14:66655367 | 69251 | 14:66916531 | 69316 | 14:68245567 | 69466 | 14:72276625 |
| 69482 | 14:72695548 | 69536 | 14:73988875 | 69639 | 14:76386785 | 69745 | 14:78754068 | 69764 | 14:79462729 |
| 69867 | 14:81514348 | 69890 | 14:81754335 | 69891 | 14:81755109 | 69932 | 15:549186 | 69952 | 15:1223740 |
| 69971 | 15:1763083 | 70074 | 15:4197179 | 70097 | 15:4603953 | 70120 | 15:5062052 | 70314 | 15:6267325 |
| 70382 | 15:7840165 | 70418 | 15:8668231 | 70426 | 15:8980586 | 70486 | 15:10462503 | 70525 | 15:11922022 |
| 70590 | 15:14196834 | 70637 | 15:15315326 | 70696 | 15:16104588 | 70710 | 15:16454917 | 70736 | 15:17101878 |
| 70782 | 15:18196851 | 70794 | 15:18357447 | 70804 | 15:18736931 | 70814 | 15:19007825 | 70838 | 15:19653453 |
| 70946 | 15:22147187 | 70997 | 15:23248430 | 71016 | 15:23621459 | 71018 | 15:23665519 | 71079 | 15:24372603 |
| 71091 | 15:24613968 | 71107 | 15:24837501 | 71123 | 15:25257089 | 71128 | 15:25308638 | 71168 | 15:26173379 |
| 71188 | 15:26573138 | 71308 | 15:28904879 | 71323 | 15:29119476 | 71357 | 15:30139511 | 71368 | 15:30396261 |
| 71408 | 15:31365259 | 71434 | 15:31780017 | 71443 | 15:32064162 | 71478 | 15:32883165 | 71517 | 15:33758501 |
| 71518 | 15:33767591 | 71580 | 15:34828367 | 71614 | 15:35688175 | 71626 | 15:35959116 | 71745 | 15:38587541 |
| 71763 | 15:38952840 | 71840 | 15:40889581 | 71862 | 15:41323112 | 71923 | 15:42472180 | 71943 | 15:42904887 |
| 72011 | 15:43795583 | 72016 | 15:43848104 | 72053 | 15:44559517 | 72177 | 15:46955071 | 72194 | 15:47343068 |
| 72237 | 15:48145511 | 72241 | 15:48207972 | 72244 | 15:48254047 | 72281 | 15:49497449 | 72308 | 15:50463550 |
| 72408 | 15:52214824 | 72429 | 15:52810027 | 72524 | 15:54359875 | 72578 | 15:55624075 | 72586 | 15:55875613 |
| 72693 | 15:57504930 | 72717 | 15:57932533 | 72747 | 15:58462557 | 72792 | 15:59699546 | 72793 | 15:59714205 |
| 72835 | 15:60939376 | 72865 | 15:61561772 | 72880 | 15:61802396 | 72881 | 15:61805665 | 72882 | 15:61806975 |
| 72885 | 15:61812433 | 72890 | 15:61960293 | 72891 | 15:61964427 | 72930 | 15:62856332 | 73076 | 15:65465351 |
| 73108 | 15:66183419 | 73192 | 15:68248786 | 73261 | 15:70440283 | 73286 | 15:71178088 | 73301 | 15:71472435 |
| 73332 | 15:72309114 | 73377 | 15:73291708 | 73393 | 15:73582243 | 73416 | 15:74163082 | 73451 | 15:74779756 |
| 73466 | 15:75089743 | 73510 | 15:76195722 | 73541 | 15:76835072 | 73630 | 15:78278239 | 73698 | 15:80309655 |
| 73754 | 15:81295713 | 73762 | 15:81438153 | 73813 | 15:82751747 | 73823 | 15:82918904 | 73880 | 15:83865721 |
| 73959 | 16:1501024 | 74086 | 16:4079477 | 74089 | 16:4114086 | 74111 | 16:4443184 | 74212 | 16:7478039 |
| 74284 | 16:10121160 | 74304 | 16:10857824 | 74327 | 16:11521662 | 74329 | 16:11621503 | 74349 | 16:12056975 |
| 74446 | 16:14409239 | 74451 | 16:14518175 | 74462 | 16:14841844 | 74479 | 16:15208552 | 74481 | 16:15279430 |
| 74498 | 16:15816016 | 74506 | 16:16013694 | 74511 | 16:16188270 | 74513 | 16:16194660 | 74578 | 16:18520385 |
| 74584 | 16:18637360 | 74591 | 16:18804747 | 74652 | 16:20427119 | 74853 | 16:25049887 | 74887 | 16:25231259 |
| 74902 | 16:25476855 | 74955 | 16:26802374 | 74956 | 16:26859375 | 74969 | 16:27035419 | 75000 | 16:27937410 |
| 75006 | 16:28023322 | 75055 | 16:29594638 | 75144 | 16:31915038 | 75148 | 16:31970232 | 75161 | 16:32424717 |
| 75174 | 16:32836939 | 75253 | 16:34714234 | 75284 | 16:35365207 | 75318 | 16:36337580 | 75342 | 16:36921696 |
| 75354 | 16:37184848 | 75457 | 16:39917217 | 75492 | 16:41063333 | 75508 | 16:41613390 | 75547 | 16:42048065 |
| 75564 | 16:42830906 | 75611 | 16:44815088 | 75636 | 16:45912914 | 75654 | 16:46353096 | 75728 | 16:48055006 |
| 75756 | 16:48627494 | 75757 | 16:48635114 | 75758 | 16:48637200 | 75759 | 16:48639734 | 75760 | 16:48645579 |
| 75761 | 16:48649527 | 75825 | 16:50275447 | 75834 | 16:50665850 | 75880 | 16:51861172 | 75887 | 16:52014418 |
| 75915 | 16:52578534 | 75975 | 16:54055090 | 75981 | 16:54147078 | 76006 | 16:54922205 | 76068 | 16:56374651 |
| 76087 | 16:56788303 | 76136 | 16:58112073 | 76149 | 16:58378141 | 76153 | 16:58406289 | 76198 | 16:59309602 |
| 76229 | 16:60126861 | 76246 | 16:60365696 | 76276 | 16:61171484 | 76282 | 16:61360408 | 76298 | 16:61585266 |
| 76320 | 16:62102938 | 76325 | 16:62226456 | 76366 | 16:63181951 | 76484 | 16:65323416 | 76487 | 16:65420109 |
| 76600 | 16:67626229 | 76631 | 16:68532430 | 76672 | 16:69359404 | 76687 | 16:69663665 | 76741 | 16:70858584 |
| 76747 | 16:70938743 | 76846 | 16:73114325 | 76890 | 16:73720962 | 76916 | 16:74372695 | 76921 | 16:74515053 |
| 77113 | 16:78293826 | 77116 | 16:78337900 | 77145 | 16:78921036 | 77151 | 16:78991296 | 77156 | 16:79028746 |
| 77158 | 16:79043302 | 77226 | 16:79983101 | 77237 | 16:80656680 | 77361 | 17:3014326 | 77369 | 17:3204260 |
| 77405 | 17:3947723 | 77459 | 17:4815417 | 77461 | 17:4875306 | 77475 | 17:5216472 | 77564 | 17:7040082 |
| 77625 | 17:8272941 | 77711 | 17:10199997 | 77712 | 17:10213106 | 78005 | 17:17163300 | 78016 | 17:17334505 |
| 78018 | 17:17357791 | 78027 | 17:17451464 | 78083 | 17:18818415 | 78126 | 17:19479964 | 78154 | 17:20220411 |
| 78164 | 17:20579995 | 78176 | 17:21008303 | 78218 | 17:22333330 | 78297 | 17:25079038 | 78310 | 17:25505355 |
| 78350 | 17:26610845 | 78351 | 17:26613473 | 78352 | 17:26635840 | 78357 | 17:26689830 | 78358 | 17:26694252 |
| 78359 | 17:26696213 | 78360 | 17:26697673 | 78362 | 17:26701612 | 78363 | 17:26731287 | 78365 | 17:26786597 |
| 78434 | 17:28547562 | 78459 | 17:29295556 | 78584 | 17:32536552 | 78587 | 17:32649823 | 78592 | 17:32740716 |
| 78597 | 17:32819809 | 78603 | 17:32962598 | 78617 | 17:33367107 | 78663 | 17:34481729 | 78680 | 17:34921588 |
| 78756 | 17:37414365 | 78774 | 17:38086662 | 78821 | 17:39604254 | 78835 | 17:39829382 | 78858 | 17:40028314 |
| 78866 | 17:40286179 | 78900 | 17:40901144 | 78909 | 17:41080474 | 78952 | 17:42386980 | 79026 | 17:44070161 |
| 79035 | 17:44280518 | 79050 | 17:44811665 | 79055 | 17:44886429 | 79088 | 17:45543757 | 79096 | 17:45774161 |
| 79102 | 17:45892895 | 79105 | 17:45908272 | 79147 | 17:46796152 | 79149 | 17:46809775 | 79150 | 17:46813078 |
| 79151 | 17:46817568 | 79152 | 17:46826871 | 79165 | 17:47298668 | 79168 | 17:47374048 | 79198 | 17:48075712 |
| 79217 | 17:48705395 | 79225 | 17:48883686 | 79261 | 17:49706199 | 79286 | 17:50270053 | 79354 | 17:51608281 |
| 79389 | 17:52115500 | 79391 | 17:52143477 | 79448 | 17:53925709 | 79467 | 17:54247909 | 79477 | 17:54494775 |
| 79546 | 17:56254970 | 79547 | 17:56259657 | 79577 | 17:57167393 | 79593 | 17:57507972 | 79621 | 17:57996907 |
| 79692 | 17:59257237 | 79708 | 17:59553144 | 79724 | 17:59967182 | 79763 | 17:60561298 | 79767 | 17:60653296 |
| 79776 | 17:60751860 | 79777 | 17:60776828 | 79791 | 17:60997432 | 79792 | 17:61002338 | 79793 | 17:61005235 |
| 79794 | 17:61005949 | 79799 | 17:61043189 | 79800 | 17:61068599 | 79804 | 17:61124602 | 79805 | 17:61125930 |
| 79806 | 17:61128578 | 79809 | 17:61149706 | 79811 | 17:61202835 | 79891 | 17:63194677 | 79894 | 17:63214725 |
| 79897 | 17:63260940 | 79924 | 17:63621804 | 79982 | 17:64851883 | 80057 | 17:66198895 | 80059 | 17:66227047 |
| 80104 | 17:67019380 | 80140 | 17:68128330 | 80158 | 17:68440573 | 80180 | 17:68951402 | 80183 | 17:68969488 |
| 80198 | 17:69224867 | 80234 | 17:70077612 | 80285 | 17:70987613 | 80306 | 17:71441658 | 80354 | 17:72302909 |
| 80450 | 18:1522517 | 80502 | 18:1802503 | 80508 | 18:1955689 | 80546 | 18:2744105 | 80574 | 18:3295457 |
| 80617 | 18:4333966 | 80629 | 18:4581539 | 80638 | 18:4763164 | 80660 | 18:5336542 | 80662 | 18:5358167 |
| 80679 | 18:5624880 | 80687 | 18:5808571 | 80698 | 18:6108723 | 80706 | 18:6166618 | 80715 | 18:6297029 |
| 80718 | 18:6328373 | 80801 | 18:7890552 | 80807 | 18:8137616 | 80811 | 18:8163590 | 80812 | 18:8166965 |
| 80813 | 18:8169505 | 80832 | 18:8512615 | 80833 | 18:8514080 | 80853 | 18:8977357 | 80855 | 18:9002587 |
| 80856 | 18:9003665 | 80857 | 18:9005002 | 80860 | 18:9032209 | 80867 | 18:9092871 | 80870 | 18:9135683 |
| 80881 | 18:9393342 | 80892 | 18:9513766 | 80943 | 18:10501656 | 80948 | 18:10593010 | 80951 | 18:10632938 |
| 80976 | 18:11155672 | 81045 | 18:12950981 | 81047 | 18:12967212 | 81076 | 18:13742613 | 81096 | 18:14219190 |
| 81124 | 18:15295023 | 81131 | 18:15355195 | 81156 | 18:16027392 | 81169 | 18:16320799 | 81178 | 18:16458903 |
| 81221 | 18:17381122 | 81234 | 18:17689118 | 81235 | 18:17739758 | 81305 | 18:19009400 | 81313 | 18:19119273 |
| 81333 | 18:19791460 | 81431 | 18:21965199 | 81444 | 18:22114555 | 81457 | 18:22311504 | 81475 | 18:22771950 |
| 81480 | 18:22863942 | 81493 | 18:23128319 | 81518 | 18:23726377 | 81540 | 18:24231814 | 81553 | 18:24599361 |
| 81590 | 18:25135273 | 81669 | 18:26830057 | 81689 | 18:27317835 | 81727 | 18:28307360 | 81772 | 18:29581458 |
| 81773 | 18:29587945 | 81783 | 18:29882243 | 81793 | 18:30199537 | 81805 | 18:30616085 | 81868 | 18:32286982 |
| 81873 | 18:32423923 | 81905 | 18:33102972 | 81907 | 18:33133073 | 81990 | 18:35092205 | 82005 | 18:35445465 |
| 82073 | 18:36962507 | 82091 | 18:37442129 | 82129 | 18:38169007 | 82139 | 18:38310010 | 82172 | 18:39170079 |
| 82266 | 18:41092657 | 82284 | 18:41476993 | 82310 | 18:42077536 | 82369 | 18:43146823 | 82379 | 18:43316323 |
| 82386 | 18:43394501 | 82412 | 18:43955990 | 82472 | 18:45795414 | 82478 | 18:45922293 | 82497 | 18:46310174 |
| 82503 | 18:46383891 | 82580 | 18:46784427 | 82591 | 18:46951310 | 82605 | 18:47321091 | 82699 | 18:49081014 |
| 82702 | 18:49135986 | 82724 | 18:49817333 | 82819 | 18:51867088 | 82926 | 18:54273698 | 82986 | 18:55259160 |
| 83048 | 18:56589786 | 83125 | 18:57434771 | 83138 | 18:57567593 | 83261 | 18:60011920 | 83313 | 18:61667250 |
| 83390 | 18:63188823 | 83451 | 18:64129214 | 83452 | 18:64134704 | 83491 | 18:64654290 | 83495 | 18:64731877 |
| 83505 | 18:64973481 | 83524 | 18:65148413 | 83569 | 18:65462851 | 83715 | 19:4421663 | 83817 | 19:6962233 |
| 83826 | 19:7152680 | 83862 | 19:7718388 | 83876 | 19:8025749 | 83881 | 19:8096180 | 83923 | 19:8635132 |
| 84010 | 19:10293568 | 84014 | 19:10327116 | 84026 | 19:10599040 | 84071 | 19:11761802 | 84072 | 19:11769174 |
| 84089 | 19:12304263 | 84170 | 19:13636619 | 84217 | 19:14667736 | 84289 | 19:16236117 | 84327 | 19:16897668 |
| 84339 | 19:17077928 | 84357 | 19:17402114 | 84369 | 19:17494959 | 84377 | 19:17756500 | 84400 | 19:18264868 |
| 84459 | 19:19573243 | 84465 | 19:19674513 | 84603 | 19:21820203 | 84607 | 19:21992770 | 84702 | 19:23904908 |
| 84739 | 19:24347524 | 84755 | 19:24595008 | 84785 | 19:24888212 | 84819 | 19:25338790 | 84846 | 19:25706233 |
| 84863 | 19:26003355 | 85007 | 19:28109982 | 85052 | 19:29119243 | 85053 | 19:29121068 | 85078 | 19:29541741 |
| 85111 | 19:30203261 | 85117 | 19:30384170 | 85124 | 19:30490687 | 85126 | 19:30515659 | 85144 | 19:30924489 |
| 85204 | 19:32369220 | 85387 | 19:35695286 | 85431 | 19:36295776 | 85435 | 19:36339138 | 85474 | 19:36999184 |
| 85492 | 19:37433519 | 85531 | 19:38678725 | 85532 | 19:38692719 | 85628 | 19:40886898 | 85633 | 19:40929680 |
| 85663 | 19:41630709 | 85664 | 19:41637084 | 85678 | 19:41804462 | 85753 | 19:43355438 | 85773 | 19:43531840 |
| 85794 | 19:44097163 | 85831 | 19:44865863 | 85905 | 19:46015589 | 85937 | 19:47051179 | 85964 | 19:47510334 |
| 85968 | 19:47580297 | 85999 | 19:48115274 | 86024 | 19:48601037 | 86035 | 19:48902647 | 86036 | 19:48931565 |
| 86071 | 19:49607062 | 86084 | 19:49887742 | 86208 | 19:51505417 | 86224 | 19:51685255 | 86249 | 19:52343433 |
| 86364 | 19:54493744 | 86401 | 19:55196487 | 86568 | 19:56726108 | 86587 | 19:57128485 | 86617 | 19:57698375 |
| 86625 | 19:58000641 | 86641 | 19:58231133 | 86813 | 19:61050416 | 86922 | 19:62757680 | 87001 | 20:973990 |
| 87030 | 20:1623518 | 87122 | 20:3090504 | 87167 | 20:4139957 | 87275 | 20:4685771 | 87328 | 20:4885163 |
| 87329 | 20:4922714 | 87330 | 20:4927232 | 87333 | 20:4936772 | 87428 | 20:5478088 | 87551 | 20:7762309 |
| 87571 | 20:8209150 | 87587 | 20:8561631 | 87601 | 20:8997165 | 87640 | 20:9653842 | 87783 | 20:12796928 |
| 87828 | 20:14014441 | 87898 | 20:16397316 | 87943 | 20:17594397 | 88015 | 20:19691235 | 88233 | 20:23492042 |
| 88295 | 20:24462304 | 88299 | 20:24489532 | 88400 | 20:27494213 | 88428 | 20:28090982 | 88464 | 20:28752172 |
| 88487 | 20:29353765 | 88516 | 20:30159496 | 88606 | 20:32897468 | 88712 | 20:35378617 | 88730 | 20:35746614 |
| 88754 | 20:36168903 | 88765 | 20:36410567 | 88793 | 20:37052070 | 88823 | 20:37587442 | 88896 | 20:39414225 |
| 88910 | 20:39546261 | 88982 | 20:41144615 | 89021 | 20:41891717 | 89030 | 20:42067151 | 89088 | 20:43711039 |
| 89200 | 20:46860182 | 89213 | 20:47186127 | 89219 | 20:47416742 | 89252 | 20:48508646 | 89386 | 20:52743029 |
| 89390 | 20:52943893 | 89391 | 20:52951946 | 89435 | 20:54078693 | 89459 | 20:54797651 | 89501 | 20:55920256 |
| 89547 | 20:56907438 | 89580 | 20:57704990 | 89603 | 20:58030736 | 89614 | 20:58246871 | 89619 | 20:58356797 |
| 89640 | 20:58731368 | 89642 | 20:58744546 | 89643 | 20:58745645 | 89644 | 20:58746207 | 89645 | 20:58747818 |
| 89646 | 20:58748682 | 89658 | 20:58932980 | 89659 | 20:58935094 | 89663 | 20:59004178 | 89666 | 20:59061779 |
| 89667 | 20:59062045 | 89674 | 20:59260188 | 89684 | 20:59558955 | 89695 | 20:59880963 | 89706 | 20:60058642 |
| 89712 | 20:60329519 | 89766 | 20:61949465 | 89781 | 20:62256568 | 89802 | 20:62677110 | 89850 | 20:63825165 |
| 89883 | 20:64360760 | 89988 | 20:66882747 | 89994 | 20:67014371 | 90009 | 20:67434867 | 90052 | 20:68158435 |
| 90064 | 20:68358778 | 90067 | 20:68381160 | 90101 | 20:68986868 | 90122 | 20:69449647 | 90149 | 20:70017030 |
| 90171 | 20:70615282 | 90299 | 21:1225541 | 90386 | 21:4129789 | 90390 | 21:4195328 | 90416 | 21:5081944 |
| 90422 | 21:5168513 | 90434 | 21:5505304 | 90441 | 21:5776117 | 90445 | 21:5799099 | 90449 | 21:5865604 |
| 90466 | 21:6422404 | 90500 | 21:7408212 | 90507 | 21:7585642 | 90558 | 21:8258222 | 90607 | 21:9489941 |
| 90625 | 21:9809810 | 90673 | 21:10967078 | 90681 | 21:11128214 | 90691 | 21:11412794 | 90733 | 21:12480366 |
| 90757 | 21:12890512 | 90783 | 21:13556460 | 90795 | 21:13850729 | 90814 | 21:14283158 | 90820 | 21:14450483 |
| 90838 | 21:14883665 | 90844 | 21:15011676 | 90955 | 21:17435585 | 90977 | 21:17918237 | 90983 | 21:18074742 |
| 91028 | 21:18934542 | 91038 | 21:19207258 | 91040 | 21:19228388 | 91042 | 21:19270043 | 91051 | 21:19367037 |
| 91086 | 21:19786899 | 91094 | 21:19865924 | 91121 | 21:20591184 | 91130 | 21:20646358 | 91220 | 21:22407987 |
| 91241 | 21:22944882 | 91282 | 21:24017992 | 91292 | 21:24359607 | 91349 | 21:26122546 | 91359 | 21:26345324 |
| 91365 | 21:26551273 | 91366 | 21:26568774 | 91378 | 21:26770078 | 91419 | 21:28090529 | 91451 | 21:29134066 |
| 91457 | 21:29367658 | 91458 | 21:29377639 | 91460 | 21:29411882 | 91490 | 21:30232035 | 91576 | 21:32380295 |
| 91580 | 21:32474563 | 91616 | 21:33229274 | 91662 | 21:33796466 | 91708 | 21:34496870 | 91781 | 21:35885347 |
| 91785 | 21:35973488 | 91802 | 21:36413785 | 91865 | 21:38466535 | 91868 | 21:38504846 | 91983 | 21:41529939 |
| 91990 | 21:41713492 | 92010 | 21:42131229 | 92100 | 21:44218636 | 92101 | 21:44266061 | 92104 | 21:44392496 |
| 92121 | 21:44681616 | 92122 | 21:44686161 | 92126 | 21:44715443 | 92130 | 21:44791184 | 92173 | 21:46307247 |
| 92174 | 21:46319841 | 92198 | 21:46784418 | 92299 | 21:48659647 | 92354 | 21:49876133 | 92365 | 21:50214540 |
| 92408 | 21:51612911 | 92464 | 21:53191938 | 92484 | 21:53761679 | 92513 | 21:54536881 | 92561 | 21:55372174 |
| 92563 | 21:55394291 | 92584 | 21:55724213 | 92598 | 21:56016789 | 92610 | 21:56129095 | 92628 | 21:56595068 |
| 92629 | 21:56609576 | 92630 | 21:56612935 | 92631 | 21:56613938 | 92632 | 21:56629591 | 92638 | 21:56817682 |
| 92639 | 21:56827233 | 92640 | 21:56835873 | 92641 | 21:56855414 | 92725 | 21:57245956 | 92790 | 21:58808829 |
| 92799 | 21:59077402 | 92811 | 21:59261689 | 92842 | 21:59841484 | 92856 | 21:59994711 | 92944 | 21:61845132 |
| 92947 | 21:61911883 | 92965 | 21:62176120 | 93011 | 21:63503056 | 93031 | 21:63983849 | 93033 | 21:64015058 |
| 93060 | 21:64546209 | 93125 | 21:65454701 | 93128 | 21:65507947 | 93165 | 21:66297320 | 93293 | 21:68995998 |
| 93474 | 22:3262622 | 93623 | 22:6202218 | 93632 | 22:6324139 | 93633 | 22:6332422 | 93693 | 22:7609231 |
| 93729 | 22:8491450 | 93766 | 22:9678468 | 93843 | 22:11157683 | 93862 | 22:11729652 | 93938 | 22:13149187 |
| 93946 | 22:13475890 | 94015 | 22:15394645 | 94026 | 22:15571331 | 94029 | 22:15621273 | 94030 | 22:15622509 |
| 94033 | 22:15666979 | 94043 | 22:15998599 | 94094 | 22:17137009 | 94106 | 22:17528025 | 94190 | 22:19523346 |
| 94236 | 22:20865658 | 94239 | 22:20892499 | 94240 | 22:20896013 | 94242 | 22:20900134 | 94248 | 22:21007069 |
| 94251 | 22:21064016 | 94261 | 22:21320700 | 94267 | 22:21445580 | 94279 | 22:21651340 | 94323 | 22:22860295 |
| 94327 | 22:22968191 | 94361 | 22:23904619 | 94386 | 22:24826985 | 94425 | 22:25962309 | 94516 | 22:28260377 |
| 94558 | 22:29032150 | 94569 | 22:29394147 | 94574 | 22:29509152 | 94575 | 22:29521016 | 94599 | 22:30171429 |
| 94614 | 22:30572708 | 94689 | 22:32709942 | 94699 | 22:32992978 | 94735 | 22:34025325 | 94738 | 22:34062706 |
| 94762 | 22:34833811 | 94770 | 22:34980123 | 94821 | 22:35930996 | 94832 | 22:36212273 | 94838 | 22:36427709 |
| 94897 | 22:37678461 | 94930 | 22:38553325 | 94957 | 22:39166570 | 94961 | 22:39195248 | 94982 | 22:39426381 |
| 94987 | 22:39550615 | 94998 | 22:39688034 | 95026 | 22:40340595 | 95073 | 22:41528657 | 95160 | 22:43561183 |
| 95169 | 22:43825654 | 95180 | 22:44046370 | 95244 | 22:45637290 | 95266 | 22:45983259 | 95343 | 22:47502907 |
| 95403 | 22:49140396 | 95406 | 22:49163015 | 95408 | 22:49233906 | 95450 | 22:50360859 | 95459 | 22:50587868 |
| 95461 | 22:50603931 | 95479 | 22:50938649 | 95519 | 22:52036264 | 95533 | 22:52343223 | 95551 | 22:52768115 |
| 95554 | 22:52841252 | 95575 | 22:53087988 | 95582 | 22:53268605 | 95650 | 22:54557160 | 95662 | 22:54685597 |
| 95702 | 22:55422111 | 95717 | 22:55662527 | 95742 | 22:55891155 | 95813 | 22:56887398 | 95816 | 22:56914900 |
| 95819 | 22:56956077 | 96032 | 23:149568 | 96113 | 23:2601798 | 96118 | 23:2653886 | 96121 | 23:2672734 |
| 96179 | 23:3852744 | 96189 | 23:3981656 | 96199 | 23:4118766 | 96251 | 23:5090449 | 96255 | 23:5153243 |
| 96280 | 23:5677678 | 96282 | 23:5716762 | 96311 | 23:6480089 | 96347 | 23:7174045 | 96353 | 23:7281266 |
| 96361 | 23:7443284 | 96593 | 23:12225865 | 96644 | 23:13147408 | 96654 | 23:13421583 | 96663 | 23:13674324 |
| 96671 | 23:13837654 | 96672 | 23:13841783 | 96674 | 23:13844745 | 96675 | 23:13850106 | 96676 | 23:13856428 |
| 96677 | 23:13860190 | 96678 | 23:13867299 | 96679 | 23:13870700 | 96680 | 23:13875916 | 96681 | 23:13885227 |
| 96708 | 23:14459570 | 96719 | 23:14657847 | 96734 | 23:14924427 | 96758 | 23:15231293 | 96802 | 23:15896256 |
| 96834 | 23:16779992 | 96854 | 23:17439199 | 96875 | 23:17872465 | 96879 | 23:17919886 | 96931 | 23:19271779 |
| 96944 | 23:19469822 | 96946 | 23:19474356 | 96988 | 23:20069380 | 97027 | 23:20827882 | 97070 | 23:21933675 |
| 97095 | 23:22572630 | 97114 | 23:23252435 | 97146 | 23:23919176 | 97176 | 23:24440913 | 97183 | 23:24589985 |
| 97237 | 23:25033379 | 97239 | 23:25047940 | 97376 | 23:27210848 | 97398 | 23:27410296 | 97713 | 23:31798120 |
| 97740 | 23:32277024 | 97758 | 23:32470121 | 97771 | 23:32757047 | 97785 | 23:33017631 | 97799 | 23:33301724 |
| 97830 | 23:34183106 | 97879 | 23:35808678 | 97919 | 23:36940417 | 97987 | 23:38818865 | 97990 | 23:38876101 |
| 98006 | 23:39270706 | 98145 | 23:40884084 | 98169 | 23:41183712 | 98196 | 23:41831694 | 98206 | 23:42044741 |
| 98238 | 23:42772846 | 98243 | 23:42890423 | 98262 | 23:43295377 | 98268 | 23:43447515 | 98302 | 23:44180408 |
| 98311 | 23:44267686 | 98338 | 23:44807132 | 98340 | 23:44830102 | 98351 | 23:45020916 | 98363 | 23:45143240 |
| 98365 | 23:45154456 | 98366 | 23:45154585 | 98367 | 23:45154695 | 98369 | 23:45170649 | 98370 | 23:45174122 |
| 98371 | 23:45194932 | 98372 | 23:45209920 | 98415 | 23:46222052 | 98487 | 23:47700808 | 98491 | 23:47790490 |
| 98613 | 23:49822048 | 98646 | 23:50347036 | 98647 | 23:50349795 | 98648 | 23:50361478 | 98695 | 23:51211166 |
| 98758 | 23:52291892 | 98780 | 24:465578 | 98795 | 24:844195 | 98914 | 24:3199425 | 99033 | 24:6106333 |
| 99089 | 24:7522593 | 99139 | 24:8885668 | 99310 | 24:11057073 | 99312 | 24:11077021 | 99344 | 24:11884545 |
| 99368 | 24:12540583 | 99376 | 24:12720792 | 99407 | 24:13477647 | 99436 | 24:14392789 | 99449 | 24:14713059 |
| 99481 | 24:15784201 | 99482 | 24:15793449 | 99520 | 24:16988712 | 99542 | 24:17540200 | 99608 | 24:19628750 |
| 99633 | 24:20394869 | 99814 | 24:23537983 | 99838 | 24:24399016 | 99917 | 24:26088773 | 99969 | 24:27563741 |
| 100022 | 24:28948556 | 100051 | 24:29641974 | 100053 | 24:29754026 | 100083 | 24:30348689 | 100125 | 24:31468489 |
| 100149 | 24:31977640 | 100204 | 24:33038149 | 100247 | 24:34170378 | 100262 | 24:34562426 | 100316 | 24:35736460 |
| 100427 | 24:39087533 | 100457 | 24:39886402 | 100506 | 24:40530911 | 100517 | 24:40707643 | 100546 | 24:41182679 |
| 100560 | 24:41739524 | 100603 | 24:42667191 | 100619 | 24:42900584 | 100623 | 24:43047337 | 100646 | 24:44068866 |
| 100667 | 24:44457109 | 100702 | 24:45155232 | 100708 | 24:45303639 | 100817 | 24:46988217 | 100842 | 24:47695031 |
| 100848 | 24:47858503 | 100861 | 24:48130487 | 100927 | 24:49987049 | 100960 | 24:50907651 | 100967 | 24:51121771 |
| 100974 | 24:51293921 | 101015 | 24:52055045 | 101040 | 24:52603746 | 101042 | 24:52612789 | 101103 | 24:54184345 |
| 101132 | 24:54726088 | 101133 | 24:54737023 | 101135 | 24:54745793 | 101174 | 24:55654866 | 101209 | 24:56364015 |
| 101214 | 24:56468941 | 101217 | 24:56488437 | 101221 | 24:56542836 | 101264 | 24:57462922 | 101329 | 24:58647028 |
| 101359 | 24:59350759 | 101409 | 24:60371452 | 101438 | 24:60938433 | 101480 | 24:61694231 | 101482 | 24:61715423 |
| 101486 | 24:61776900 | 101548 | 25:1123694 | 101554 | 25:1211629 | 101568 | 25:1368623 | 101741 | 25:2890918 |
| 101757 | 25:3291302 | 101761 | 25:3373790 | 101762 | 25:3388313 | 101779 | 25:3563948 | 101807 | 25:4175576 |
| 101833 | 25:4621429 | 101909 | 25:5758755 | 101916 | 25:5934892 | 101922 | 25:6041488 | 101970 | 25:7127243 |
| 102009 | 25:7910528 | 102017 | 25:8111580 | 102058 | 25:9294790 | 102089 | 25:9965727 | 102096 | 25:10068090 |
| 102163 | 25:11303404 | 102165 | 25:11364705 | 102181 | 25:11635729 | 102182 | 25:11657253 | 102214 | 25:12395830 |
| 102217 | 25:12420213 | 102227 | 25:12757945 | 102253 | 25:13410180 | 102261 | 25:13589337 | 102275 | 25:14025698 |
| 102289 | 25:14337887 | 102372 | 25:16006571 | 102395 | 25:16397106 | 102396 | 25:16427094 | 102524 | 25:19554407 |
| 102537 | 25:19935329 | 102562 | 25:20762754 | 102600 | 25:21483496 | 102602 | 25:21510337 | 102641 | 25:22365508 |
| 102653 | 25:22668990 | 102699 | 25:23839896 | 102714 | 25:24079936 | 102736 | 25:24542279 | 102742 | 25:24617636 |
| 102767 | 25:25119747 | 102769 | 25:25147993 | 102791 | 25:25378579 | 102816 | 25:26015769 | 102932 | 25:28026271 |
| 102975 | 25:29198572 | 102994 | 25:29627243 | 103031 | 25:30198863 | 103080 | 25:31339867 | 103118 | 25:32018635 |
| 103199 | 25:33663856 | 103229 | 25:34356818 | 103305 | 25:35625080 | 103338 | 25:36046363 | 103342 | 25:36137423 |
| 103378 | 25:36442406 | 103457 | 25:37592711 | 103460 | 25:37631841 | 103479 | 25:38076102 | 103536 | 25:39097677 |
| 103596 | 25:40251086 | 103616 | 25:40630329 | 103683 | 25:41733621 | 103706 | 26:19998 | 103766 | 26:2270743 |
| 103818 | 26:3188174 | 103819 | 26:3191375 | 103829 | 26:3361204 | 103909 | 26:5551721 | 104006 | 26:8275246 |
| 104020 | 26:8585213 | 104105 | 26:10587007 | 104135 | 26:11460715 | 104155 | 26:11751995 | 104195 | 26:13034814 |
| 104284 | 26:14982249 | 104298 | 26:15453105 | 104410 | 26:18118254 | 104413 | 26:18261377 | 104460 | 26:19187481 |
| 104469 | 26:19395441 | 104471 | 26:19411437 | 104493 | 26:19971593 | 104520 | 26:20855927 | 104538 | 26:21270336 |
| 104539 | 26:21271111 | 104540 | 26:21272246 | 104542 | 26:21276672 | 104554 | 26:21609152 | 104556 | 26:21638374 |
| 104557 | 26:21659298 | 104575 | 26:22193698 | 104576 | 26:22197941 | 104651 | 26:24350779 | 104659 | 26:24607752 |
| 104686 | 26:24921440 | 104693 | 26:25131752 | 104762 | 26:26169541 | 104768 | 26:26273875 | 104774 | 26:26426669 |
| 104788 | 26:26770718 | 104860 | 26:28504622 | 104867 | 26:28761933 | 104935 | 26:30753391 | 104952 | 26:31231421 |
| 104953 | 26:31240168 | 105002 | 26:32486347 | 105010 | 26:32645140 | 105037 | 26:33152552 | 105049 | 26:33514228 |
| 105055 | 26:33591046 | 105060 | 26:33741604 | 105081 | 26:34086479 | 105174 | 26:36209430 | 105228 | 26:37411119 |
| 105255 | 26:38327739 | 105259 | 26:38422867 | 105266 | 26:38582767 | 105287 | 26:39039410 | 105319 | 26:39645163 |
| 105321 | 26:39706475 | 105436 | 26:42682884 | 105480 | 26:43924743 | 105484 | 26:44008043 | 105557 | 26:45531818 |
| 105583 | 26:46019051 | 105611 | 26:46544547 | 105633 | 26:47031869 | 105741 | 26:49412031 | 105763 | 26:49860726 |
| 105823 | 26:51197832 | 105827 | 26:51273644 | 105828 | 26:51339616 | 105832 | 26:51408594 | 105941 | 27:2266510 |
| 105949 | 27:2424175 | 106008 | 27:3926602 | 106025 | 27:4205889 | 106087 | 27:5556009 | 106166 | 27:7956657 |
| 106176 | 27:8174752 | 106200 | 27:8748506 | 106216 | 27:9098489 | 106217 | 27:9098570 | 106218 | 27:9098663 |
| 106219 | 27:9099087 | 106220 | 27:9099279 | 106221 | 27:9099292 | 106222 | 27:9099380 | 106223 | 27:9099465 |
| 106224 | 27:9099696 | 106225 | 27:9099832 | 106226 | 27:9104046 | 106228 | 27:9106497 | 106230 | 27:9110724 |
| 106231 | 27:9116774 | 106232 | 27:9117417 | 106233 | 27:9117873 | 106234 | 27:9117874 | 106235 | 27:9118114 |
| 106236 | 27:9118136 | 106237 | 27:9118300 | 106240 | 27:9149222 | 106241 | 27:9151257 | 106256 | 27:9578023 |
| 106335 | 27:12004591 | 106383 | 27:13309317 | 106461 | 27:14313738 | 106474 | 27:14529862 | 106519 | 27:15481603 |
| 106554 | 27:16106255 | 106656 | 27:18041339 | 106668 | 27:18444933 | 106677 | 27:18618418 | 106687 | 27:18831735 |
| 106688 | 27:18844548 | 106691 | 27:18895158 | 106692 | 27:18916416 | 106693 | 27:18921032 | 106756 | 27:20229425 |
| 106790 | 27:21326986 | 106801 | 27:21699196 | 106805 | 27:21744178 | 106817 | 27:21948844 | 106848 | 27:22823774 |
| 106884 | 27:23610622 | 106908 | 27:24137880 | 107014 | 27:26432887 | 107082 | 27:27644914 | 107095 | 27:27964245 |
| 107202 | 27:30418455 | 107334 | 27:32927582 | 107343 | 27:33025196 | 107408 | 27:34899643 | 107448 | 27:35233200 |
| 107456 | 27:35414868 | 107653 | 27:39221884 | 107712 | 27:40883338 | 107746 | 27:41747888 | 107759 | 27:42126889 |
| 107801 | 27:43079592 | 107803 | 27:43088850 | 107843 | 27:43939855 | 107846 | 27:43979622 | 107847 | 27:43983990 |
| 107854 | 27:44107624 | 107862 | 27:44229578 | 107939 | 28:1012326 | 107958 | 28:1304486 | 107974 | 28:1564870 |
| 108000 | 28:2124177 | 108040 | 28:3650698 | 108041 | 28:3666554 | 108059 | 28:4687863 | 108061 | 28:4710151 |
| 108084 | 28:5241897 | 108148 | 28:6765529 | 108153 | 28:6864730 | 108232 | 28:8673885 | 108245 | 28:9022551 |
| 108246 | 28:9025349 | 108267 | 28:9565874 | 108279 | 28:9801762 | 108289 | 28:9992581 | 108310 | 28:10520846 |
| 108311 | 28:10565818 | 108389 | 28:12680409 | 108396 | 28:12751017 | 108476 | 28:14911107 | 108581 | 28:17140257 |
| 108632 | 28:18044696 | 108660 | 28:18836541 | 108739 | 28:20933740 | 108741 | 28:20941251 | 108783 | 28:22351391 |
| 108826 | 28:23326980 | 108859 | 28:24154609 | 108866 | 28:24223951 | 108903 | 28:25048959 | 108912 | 28:25216858 |
| 109001 | 28:26844276 | 109007 | 28:27009355 | 109043 | 28:27784145 | 109052 | 28:27909599 | 109054 | 28:27910886 |
| 109073 | 28:28015394 | 109215 | 28:31286284 | 109245 | 28:32003701 | 109325 | 28:33230710 | 109348 | 28:33703563 |
| 109406 | 28:34803927 | 109413 | 28:34924460 | 109417 | 28:34965171 | 109472 | 28:36153178 | 109522 | 28:37465397 |
| 109570 | 28:38512629 | 109579 | 28:38669301 | 109622 | 28:39347343 | 109640 | 28:39934529 | 109726 | 28:40743505 |
| 109728 | 28:40802700 | 109729 | 28:40815314 | 109735 | 28:40867971 | 109741 | 28:40986070 | 109745 | 28:41069948 |
| 109774 | 28:41574166 | 109825 | 28:42451978 | 109871 | 28:43255478 | 109915 | 28:44119755 | 109971 | 28:45223304 |
| 110037 | 29:1129729 | 110066 | 29:1630821 | 110088 | 29:2215375 | 110115 | 29:2538458 | 110120 | 29:2640739 |
| 110159 | 29:3485853 | 110162 | 29:3535737 | 110211 | 29:4767448 | 110325 | 29:8136563 | 110346 | 29:8758233 |
| 110486 | 29:11888737 | 110531 | 29:12717025 | 110539 | 29:12922913 | 110611 | 29:14988640 | 110619 | 29:15140865 |
| 110686 | 29:16917842 | 110688 | 29:16949756 | 110701 | 29:17144041 | 110705 | 29:17304932 | 110731 | 29:18241209 |
| 110749 | 29:19246346 | 110842 | 29:21593668 | 110893 | 29:23085229 | 110896 | 29:23104998 | 110900 | 29:23212423 |
| 110941 | 29:24050623 | 111030 | 29:26373975 | 111053 | 29:27005732 | 111066 | 29:27532899 | 111074 | 29:27658327 |
| 111146 | 29:29021609 | 111151 | 29:29269330 | 111156 | 29:29356340 | 111204 | 29:29930474 | 111235 | 29:30673349 |
| 111259 | 29:31276001 | 111275 | 29:31521382 | 111281 | 29:31682726 | 111359 | 29:33581781 | 111361 | 29:33604607 |
| 111377 | 29:33936893 | 111379 | 29:33963898 | 111395 | 29:34390629 | 111404 | 29:34569090 | 111415 | 29:34694545 |
| 111459 | 29:35661091 | 111465 | 29:35722342 | 111509 | 29:36453498 | 111527 | 29:36872914 | 111613 | 29:39811611 |
| 111614 | 29:39822689 | 111712 | 29:41607581 | 111724 | 29:41814129 | 111745 | 29:42165191 | 111770 | 29:42669048 |
| 111788 | 29:43153261 | 111839 | 29:43881785 | 111856 | 29:44096512 | 111873 | 29:44366521 | 111948 | 29:46176719 |
| 111954 | 29:46281096 | 112022 | 29:47700163 | 112024 | 29:47731596 | 112148 | 29:50449063 | | |

In the present disclosure, a fifth marker panel was preferably a total of 6,190 functional SNPs related to beef cattle traits obtained from public databases. The acquisition of the fifth marker panel preferably included: currently known quantitative trait loci (QTL) and SNPs data of bovine species were downloaded from the online animal QTL database (https://www.animalgenome.org/cgi-bin/QTLdb/BT/index). A total of 7,763 QTLs related to five major traits of beef cattle, including exterior, health, meat and carcass, production, reproduction, and the included SNPs information were obtained. According to the rs number of each marker, their corresponding physical positions in the “ARS-UCD 1.2/bosTau9” genome version were found, and a total of 6,190 SNPs with MAF>0.05 in Huaxi cattle population were selected. The information of fifth marker panel was shown in Table 5:

TABLE 5
Location information of fifth marker panel

| NO. | Position | NO. | Position | NO. | Position | NO. | Position | NO. | Position |
|---|---|---|---|---|---|---|---|---|---|
| 36 | 1:1247889 | 37 | 1:1248205 | 38 | 1:1248699 | 39 | 1:1252437 | 46 | 1:1417011 |
| 47 | 1:1438871 | 48 | 1:1439812 | 51 | 1:1473953 | 52 | 1:1474233 | 53 | 1:1505639 |
| 54 | 1:1517594 | 55 | 1:1518088 | 56 | 1:1523007 | 57 | 1:1524793 | 58 | 1:1540344 |
| 59 | 1:1540422 | 60 | 1:1540458 | 61 | 1:1540555 | 119 | 1:2769164 | 139 | 1:3245141 |
| 144 | 1:3303269 | 169 | 1:3679697 | 170 | 1:3679721 | 171 | 1:3687775 | 185 | 1:3908027 |
| 207 | 1:4250046 | 209 | 1:4276065 | 400 | 1:8645661 | 446 | 1:9625272 | 607 | 1:13657759 |
| 715 | 1:16486738 | 725 | 1:16754495 | 738 | 1:17263605 | 750 | 1:17537664 | 784 | 1:18789066 |
| 935 | 1:23001427 | 965 | 1:23946108 | 991 | 1:24758865 | 993 | 1:24782983 | 1041 | 1:26105452 |
| 1093 | 1:27491647 | 1117 | 1:28566525 | 1118 | 1:28612680 | 1152 | 1:29520198 | 1205 | 1:31082779 |
| 1284 | 1:33805826 | 1294 | 1:33964232 | 1471 | 1:38630217 | 1603 | 1:42156367 | 1607 | 1:42205645 |
| 1646 | 1:43258942 | 1650 | 1:43390633 | 1777 | 1:46016435 | 1879 | 1:49422581 | 1953 | 1:51785197 |
| 1964 | 1:52229363 | 1967 | 1:52281229 | 2171 | 1:57147119 | 2172 | 1:57180246 | 2296 | 1:60247154 |
| 2297 | 1:60275851 | 2332 | 1:61100002 | 2339 | 1:61219155 | 2362 | 1:61796684 | 2462 | 1:64193131 |
| 2469 | 1:64272382 | 2530 | 1:65583709 | 2532 | 1:65625274 | 2535 | 1:65763047 | 2669 | 1:69022641 |
| 2694 | 1:69479795 | 2715 | 1:69942383 | 2774 | 1:70823693 | 2916 | 1:74460838 | 3081 | 1:78554220 |
| 3121 | 1:79513248 | 3351 | 1:85398326 | 3352 | 1:85456698 | 3354 | 1:85501954 | 3382 | 1:86229426 |
| 3387 | 1:86366405 | 3411 | 1:87047388 | 3436 | 1:87762016 | 3496 | 1:89623272 | 3548 | 1:90545947 |
| 3594 | 1:91706588 | 3654 | 1:93372952 | 3656 | 1:93477242 | 3826 | 1:97778617 | 3827 | 1:97802384 |
| 3832 | 1:97878133 | 3860 | 1:98721777 | 3895 | 1:99586706 | 4190 | 1:108556096 | 4194 | 1:108577099 |
| 4350 | 1:112307236 | 4383 | 1:112837296 | 4428 | 1:113860732 | 4434 | 1:114102926 | 4466 | 1:114828912 |
| 4467 | 1:114837165 | 4530 | 1:116786787 | 4560 | 1:117406892 | 4601 | 1:118129247 | 4619 | 1:118286166 |
| 4638 | 1:118603687 | 4640 | 1:118638525 | 4644 | 1:118785100 | 4645 | 1:118808555 | 4676 | 1:119387495 |
| 4706 | 1:120231880 | 4757 | 1:121536001 | 4852 | 1:124087395 | 4901 | 1:125202968 | 4921 | 1:125609019 |
| 4991 | 1:127321191 | 5033 | 1:128301452 | 5034 | 1:128336208 | 5132 | 1:130745923 | 5143 | 1:131052225 |
| 5212 | 1:131617330 | 5252 | 1:132852921 | 5255 | 1:132924609 | 5257 | 1:132963363 | 5326 | 1:134972795 |
| 5386 | 1:135785234 | 5387 | 1:135785949 | 5388 | 1:135787843 | 5389 | 1:135788916 | 5390 | 1:135789433 |
| 5391 | 1:135791112 | 5448 | 1:137501805 | 5548 | 1:140642688 | 5607 | 1:141638716 | 5670 | 1:142753326 |
| 5736 | 1:143879207 | 5833 | 1:145815334 | 5857 | 1:146000862 | 5863 | 1:146077457 | 5868 | 1:146242466 |
| 5876 | 1:146437585 | 5882 | 1:146612774 | 5891 | 1:146975346 | 5965 | 1:148149815 | 6115 | 1:150888684 |
| 6274 | 1:153150228 | 6320 | 1:153989230 | 6340 | 1:154314444 | 6342 | 1:154371107 | 6343 | 1:154385540 |
| 6547 | 2:682101 | 6574 | 2:1161894 | 6626 | 2:2113211 | 6636 | 2:2405667 | 6699 | 2:4200142 |
| 6781 | 2:5668859 | 6810 | 2:6248437 | 6811 | 2:6253061 | 6813 | 2:6269804 | 6815 | 2:6275413 |
| 6834 | 2:6717203 | 6835 | 2:6717548 | 6836 | 2:6732409 | 6837 | 2:6732955 | 6873 | 2:7379901 |
| 6888 | 2:7706163 | 6911 | 2:8334857 | 6932 | 2:8969249 | 7019 | 2:11681348 | 7024 | 2:11775775 |
| 7049 | 2:12802862 | 7070 | 2:13346155 | 7119 | 2:14632486 | 7120 | 2:14676143 | 7125 | 2:14743753 |
| 7148 | 2:15330545 | 7152 | 2:15479415 | 7159 | 2:15620578 | 7175 | 2:15995516 | 7188 | 2:16261766 |
| 7244 | 2:17777834 | 7245 | 2:17826748 | 7280 | 2:18272965 | 7294 | 2:18380242 | 7300 | 2:18552906 |
| 7374 | 2:20073035 | 7420 | 2:21391926 | 7484 | 2:23026213 | 7579 | 2:24732559 | 7750 | 2:28419342 |
| 7751 | 2:28419366 | 7752 | 2:28421072 | 8028 | 2:35888381 | 8230 | 2:41375205 | 8293 | 2:42660415 |
| 8319 | 2:43375192 | 8449 | 2:46777670 | 8450 | 2:46782605 | 8452 | 2:46797429 | 8466 | 2:47070734 |
| 8467 | 2:47100164 | 8549 | 2:49214428 | 8560 | 2:49760042 | 8561 | 2:49760735 | 8735 | 2:54491736 |
| 8791 | 2:55960682 | 8792 | 2:55979085 | 9079 | 2:63568686 | 9094 | 2:64064953 | 9161 | 2:65602756 |
| 9288 | 2:69830690 | 9347 | 2:71285767 | 9373 | 2:72193932 | 9374 | 2:72216636 | 9516 | 2:76002159 |
| 9530 | 2:76376460 | 9555 | 2:76976290 | 9786 | 2:83877880 | 9791 | 2:84089700 | 10121 | 2:91931085 |
| 10139 | 2:92377009 | 10180 | 2:93321855 | 10182 | 2:93326125 | 10183 | 2:93326273 | 10184 | 2:93327304 |
| 10185 | 2:93353835 | 10186 | 2:93353877 | 10187 | 2:93354446 | 10188 | 2:93354619 | 10189 | 2:93354804 |
| 10190 | 2:93355262 | 10191 | 2:93355271 | 10192 | 2:93356138 | 10193 | 2:93356208 | 10194 | 2:93356223 |
| 10195 | 2:93366311 | 10248 | 2:94636270 | 10286 | 2:95449694 | 10305 | 2:95979209 | 10312 | 2:96139223 |
| 10401 | 2:98337087 | 10409 | 2:98421675 | 10413 | 2:98461459 | 10417 | 2:98536760 | 10466 | 2:99940359 |
| 10605 | 2:103776260 | 10655 | 2:105009833 | 10686 | 2:105635824 | 10694 | 2:105828123 | 10712 | 2:106230731 |
| 10781 | 2:107728454 | 10782 | 2:107728517 | 10783 | 2:107729520 | 10784 | 2:107730636 | 10785 | 2:107735095 |
| 10786 | 2:107735449 | 10787 | 2:107737533 | 10789 | 2:107742839 | 10800 | 2:107971542 | 10846 | 2:109019023 |
| 10929 | 2:111354781 | 10934 | 2:111635526 | 10954 | 2:112098945 | 10956 | 2:112105210 | 10975 | 2:112632558 |
| 10978 | 2:112654694 | 11038 | 2:113914830 | 11099 | 2:115533946 | 11303 | 2:120430113 | 11405 | 2:123285701 |
| 11444 | 2:124279927 | 11460 | 2:124841893 | 11684 | 2:128718214 | 11813 | 2:131557054 | 11822 | 2:131862099 |
| 11852 | 2:132351992 | 11940 | 2:133852858 | 11943 | 2:133878448 | 11959 | 2:134115936 | 11960 | 2:134172533 |
| 12004 | 2:134750704 | 12059 | 2:135525484 | 12143 | 3:950659 | 12145 | 3:954886 | 12187 | 3:1728119 |
| 12274 | 3:3596795 | 12343 | 3:4727841 | 12452 | 3:6832814 | 12454 | 3:6841596 | 12456 | 3:6848201 |
| 12457 | 3:6866175 | 12458 | 3:6867557 | 12459 | 3:6867867 | 12460 | 3:6885163 | 12533 | 3:8025174 |
| 12734 | 3:11445314 | 12790 | 3:12934994 | 12856 | 3:14226434 | 12920 | 3:15616501 | 12948 | 3:15994930 |
| 13048 | 3:18771436 | 13049 | 3:18775018 | 13138 | 3:21163145 | 13156 | 3:21764453 | 13190 | 3:22332383 |
| 13191 | 3:22334033 | 13192 | 3:22339164 | 13193 | 3:22340631 | 13194 | 3:22343298 | 13357 | 3:26160439 |
| 13470 | 3:27968845 | 13475 | 3:28047336 | 13527 | 3:29246450 | 13584 | 3:30235042 | 13600 | 3:30605190 |
| 13623 | 3:31149250 | 13660 | 3:31948925 | 13662 | 3:31976577 | 13736 | 3:32970665 | 13765 | 3:33258971 |
| 14049 | 3:40032182 | 14051 | 3:40065534 | 14053 | 3:40120645 | 14188 | 3:43541601 | 14331 | 3:47791771 |
| 14354 | 3:48191758 | 14355 | 3:48234094 | 14392 | 3:49000989 | 14416 | 3:49459128 | 14422 | 3:49526251 |
| 14428 | 3:49574985 | 14483 | 3:49988687 | 14504 | 3:50656944 | 14581 | 3:52783346 | 14661 | 3:55239885 |
| 14665 | 3:55330131 | 14835 | 3:60375726 | 14845 | 3:60575455 | 14850 | 3:60636353 | 14868 | 3:60938213 |
| 14958 | 3:63728694 | 14960 | 3:63776877 | 14965 | 3:63861865 | 14966 | 3:63887795 | 14968 | 3:63950495 |
| 14969 | 3:63955495 | 14970 | 3:63976119 | 14973 | 3:64020621 | 14975 | 3:64058308 | 14983 | 3:64239387 |
| 14988 | 3:64525583 | 14992 | 3:64697961 | 15033 | 3:65670943 | 15044 | 3:66042478 | 15049 | 3:66148244 |
| 15107 | 3:67465318 | 15130 | 3:67947626 | 15131 | 3:67978806 | 15150 | 3:68443615 | 15175 | 3:69034450 |
| 15230 | 3:70652398 | 15234 | 3:70860935 | 15243 | 3:71230722 | 15285 | 3:72930619 | 15393 | 3:76153357 |
| 15442 | 3:77599781 | 15443 | 3:77635657 | 15446 | 3:77784239 | 15495 | 3:79128870 | 15498 | 3:79224313 |
| 15538 | 3:80529583 | 15563 | 3:81048556 | 15605 | 3:82166845 | 15625 | 3:82581418 | 15684 | 3:84280062 |
| 15939 | 3:89312804 | 15941 | 3:89315922 | 16034 | 3:91258808 | 16253 | 3:96974610 | 16259 | 3:97049079 |
| 16287 | 3:97630051 | 16414 | 3:100563623 | 16430 | 3:100751713 | 16431 | 3:100782663 | 16443 | 3:101112794 |
| 16446 | 3:101150016 | 16537 | 3:102883747 | 16541 | 3:102931082 | 16598 | 3:104333611 | 16602 | 3:104484207 |
| 16605 | 3:104506752 | 16607 | 3:104541890 | 16609 | 3:104556518 | 16628 | 3:104869949 | 16646 | 3:105209686 |
| 16654 | 3:105292781 | 16714 | 3:105965567 | 16715 | 3:105973059 | 16718 | 3:106026621 | 16719 | 3:106042336 |
| 16740 | 3:106492826 | 16750 | 3:106599058 | 16901 | 3:109689309 | 17056 | 3:113011961 | 17068 | 3:113301217 |
| 17115 | 3:113990093 | 17186 | 3:114897383 | 17380 | 3:117882488 | 17384 | 3:117909372 | 17401 | 3:118223118 |
| 17402 | 3:118253078 | 17639 | 4:4003154 | 17644 | 4:4149740 | 17697 | 4:5004514 | 17705 | 4:5165078 |
| 17706 | 4:5173632 | 17740 | 4:5747787 | 17752 | 4:6130688 | 17761 | 4:6330228 | 17968 | 4:10578187 |
| 17969 | 4:10580814 | 18198 | 4:16670558 | 18273 | 4:18595000 | 18276 | 4:18619515 | 18318 | 4:19200647 |
| 18378 | 4:20303118 | 18635 | 4:26901086 | 18636 | 4:26934291 | 18820 | 4:32010653 | 18843 | 4:32768824 |
| 18949 | 4:35971843 | 19100 | 4:40158341 | 19134 | 4:40946237 | 19168 | 4:41815635 | 19173 | 4:42048079 |
| 19208 | 4:43120197 | 19214 | 4:43203876 | 19250 | 4:44121669 | 19285 | 4:45161683 | 19356 | 4:47190294 |
| 19377 | 4:47463756 | 19400 | 4:48100380 | 19424 | 4:48952233 | 19466 | 4:49461342 | 19477 | 4:49813182 |
| 19478 | 4:49842196 | 19480 | 4:49888592 | 19866 | 4:61781138 | 19888 | 4:62276469 | 19891 | 4:62357256 |
| 19931 | 4:63512286 | 19965 | 4:64527362 | 19982 | 4:64979970 | 20028 | 4:66103436 | 20075 | 4:67374218 |
| 20136 | 4:69236841 | 20289 | 4:73424213 | 20513 | 4:78984643 | 20553 | 4:80105286 | 20554 | 4:80107022 |
| 20555 | 4:80107183 | 20556 | 4:80107305 | 20557 | 4:80107320 | 20558 | 4:80107386 | 20559 | 4:80107434 |
| 20560 | 4:80107694 | 20561 | 4:80107717 | 20562 | 4:80107759 | 20674 | 4:82773442 | 20681 | 4:82863329 |
| 20749 | 4:85034221 | 20779 | 4:85832979 | 20780 | 4:85866052 | 20788 | 4:85968886 | 20834 | 4:87033808 |
| 20882 | 4:88417629 | 20886 | 4:88515168 | 20903 | 4:89049127 | 21046 | 4:92398977 | 21050 | 4:92448883 |
| 21051 | 4:92451008 | 21052 | 4:92451165 | 21109 | 4:93517701 | 21117 | 4:93652019 | 21185 | 4:95523178 |
| 21553 | 4:100762424 | 21603 | 4:101689426 | 21683 | 4:103739455 | 21711 | 4:104521615 | 21718 | 4:104699947 |
| 21818 | 4:106683233 | 21824 | 4:106777370 | 21836 | 4:106986917 | 21886 | 4:107625627 | 21968 | 4:109840544 |
| 22030 | 4:111174187 | 22078 | 4:111934020 | 22116 | 4:112499124 | 22123 | 4:112606237 | 22134 | 4:112692746 |
| 22137 | 4:112719377 | 22140 | 4:112741810 | 22149 | 4:112797073 | 22180 | 4:113503432 | 22250 | 4:114401519 |
| 22253 | 4:114443077 | 22274 | 4:114788184 | 22278 | 4:114812550 | 22312 | 4:115192347 | 22318 | 4:115316340 |
| 22319 | 4:115317642 | 22354 | 4:116067653 | 22376 | 4:116420553 | 22394 | 4:116749463 | 22406 | 4:116987390 |
| 22513 | 4:119160825 | 22525 | 4:119536985 | 22581 | 5:882265 | 22670 | 5:2514974 | 22693 | 5:3086065 |
| 22707 | 5:3684231 | 22731 | 5:4371208 | 22740 | 5:4517412 | 22761 | 5:5025515 | 22776 | 5:5256216 |
| 22899 | 5:8017094 | 22935 | 5:8963886 | 22937 | 5:9058312 | 22939 | 5:9081371 | 22941 | 5:9110521 |
| 22974 | 5:9917938 | 23184 | 5:14901254 | 23203 | 5:15246344 | 23335 | 5:18879061 | 23340 | 5:18957429 |
| 23448 | 5:21803211 | 23511 | 5:22947860 | 23513 | 5:23010158 | 23563 | 5:23986445 | 23619 | 5:25424207 |
| 23628 | 5:25663032 | 23683 | 5:27040938 | 23685 | 5:27062257 | 23767 | 5:28682732 | 23795 | 5:29481439 |
| 23894 | 5:31935957 | 23904 | 5:32259633 | 23908 | 5:32334842 | 23922 | 5:32649736 | 23951 | 5:33311326 |
| 24015 | 5:35004932 | 24046 | 5:36022885 | 24051 | 5:36113810 | 24053 | 5:36193854 | 24092 | 5:37455345 |
| 24105 | 5:37749836 | 24243 | 5:41293748 | 24244 | 5:41337074 | 24247 | 5:41451300 | 24254 | 5:41682610 |
| 24265 | 5:41958597 | 24268 | 5:42010622 | 24275 | 5:42382038 | 24295 | 5:42883310 | 24301 | 5:43024524 |
| 24312 | 5:43371061 | 24352 | 5:44118721 | 24387 | 5:45222817 | 24450 | 5:46743902 | 24683 | 5:52698528 |
| 24690 | 5:53009069 | 25020 | 5:61964739 | 25024 | 5:62044690 | 25041 | 5:62542053 | 25049 | 5:62915639 |
| 25350 | 5:69917738 | 25398 | 5:71197728 | 25467 | 5:72417962 | 25469 | 5:72465277 | 25527 | 5:74079648 |
| 25639 | 5:76766433 | 25642 | 5:76818912 | 25644 | 5:76890153 | 25645 | 5:76972053 | 25757 | 5:80114008 |
| 25760 | 5:80235852 | 25784 | 5:80982109 | 25787 | 5:81116016 | 25814 | 5:81935764 | 25875 | 5:83029733 |
| 26074 | 5:84527341 | 26206 | 5:87607418 | 26217 | 5:87838676 | 26304 | 5:89147995 | 26364 | 5:90614584 |
| 26395 | 5:91363881 | 26508 | 5:93490294 | 26558 | 5:94637080 | 26603 | 5:95645793 | 26605 | 5:95684692 |
| 26607 | 5:95699816 | 26610 | 5:95747447 | 26611 | 5:95748288 | 26612 | 5:95749996 | 26763 | 5:98601044 |
| 26855 | 5:100383163 | 26876 | 5:100815761 | 26879 | 5:100854950 | 26917 | 5:101625777 | 27085 | 5:105474132 |
| 27094 | 5:105732916 | 27095 | 5:105733857 | 27096 | 5:105740050 | 27097 | 5:105756724 | 27098 | 5:105760018 |
| 27099 | 5:105760750 | 27101 | 5:105770267 | 27102 | 5:105771805 | 27103 | 5:105773809 | 27104 | 5:105776080 |
| 27105 | 5:105776475 | 27106 | 5:105778521 | 27107 | 5:105778777 | 27108 | 5:105779368 | 27109 | 5:105780258 |
| 27110 | 5:105800847 | 27112 | 5:105804880 | 27167 | 5:106540440 | 27175 | 5:106650676 | 27227 | 5:107423031 |
| 27378 | 5:110192579 | 27381 | 5:110289240 | 27391 | 5:110386083 | 27419 | 5:110751512 | 27501 | 5:112519431 |
| 27502 | 5:112553713 | 27533 | 5:113205459 | 27568 | 5:114190671 | 27822 | 5:117930669 | 27862 | 5:118720245 |
| 27891 | 5:119036022 | 27910 | 5:119441240 | 28029 | 6:1993838 | 28117 | 6:3589994 | 28156 | 6:4424160 |
| 28249 | 6:6458237 | 28250 | 6:6482619 | 28394 | 6:10017733 | 28447 | 6:11463553 | 28470 | 6:11875946 |
| 28484 | 6:12191468 | 28503 | 6:12548297 | 28504 | 6:12550413 | 28512 | 6:12753537 | 28513 | 6:12808531 |
| 28515 | 6:12873396 | 28517 | 6:12900652 | 28580 | 6:14515552 | 28582 | 6:14546752 | 28584 | 6:14593717 |
| 28588 | 6:14737560 | 28660 | 6:16069555 | 28665 | 6:16173201 | 28670 | 6:16334539 | 28671 | 6:16387845 |
| 28672 | 6:16407876 | 28700 | 6:16918116 | 28708 | 6:17036127 | 28721 | 6:17373915 | 28727 | 6:17498903 |
| 28786 | 6:18598550 | 28800 | 6:18917943 | 28802 | 6:18955466 | 28828 | 6:19455356 | 28838 | 6:19628808 |
| 28847 | 6:19848821 | 28851 | 6:19923403 | 28858 | 6:20135992 | 28864 | 6:20273699 | 28873 | 6:20399602 |
| 28881 | 6:20600966 | 28884 | 6:20625823 | 28891 | 6:20779771 | 28892 | 6:20821807 | 28906 | 6:21288355 |
| 28909 | 6:21362764 | 28911 | 6:21407401 | 28920 | 6:21614174 | 28924 | 6:21682567 | 28929 | 6:21732184 |
| 28930 | 6:21736509 | 28937 | 6:21857115 | 28943 | 6:22080199 | 28948 | 6:22169669 | 28958 | 6:22265776 |
| 28982 | 6:22743978 | 28986 | 6:22838838 | 29009 | 6:23503327 | 29017 | 6:23665616 | 29019 | 6:23697957 |
| 29025 | 6:23885602 | 29026 | 6:23902363 | 29027 | 6:23902790 | 29037 | 6:24156443 | 29038 | 6:24188452 |
| 29062 | 6:24764063 | 29073 | 6:25006732 | 29074 | 6:25007314 | 29075 | 6:25007705 | 29085 | 6:25188129 |
| 29088 | 6:25333843 | 29095 | 6:25528726 | 29104 | 6:25843075 | 29112 | 6:25978766 | 29117 | 6:26022919 |
| 29125 | 6:26158019 | 29142 | 6:26679658 | 29146 | 6:26760274 | 29148 | 6:26792379 | 29156 | 6:27042457 |
| 29160 | 6:27154761 | 29165 | 6:27332801 | 29171 | 6:27406476 | 29192 | 6:28048764 | 29202 | 6:28285173 |
| 29219 | 6:28873303 | 29221 | 6:28936994 | 29222 | 6:28940832 | 29224 | 6:28978809 | 29229 | 6:29062262 |
| 29242 | 6:29357249 | 29246 | 6:29406846 | 29252 | 6:29478697 | 29261 | 6:29591601 | 29264 | 6:29642427 |
| 29266 | 6:29700842 | 29272 | 6:29850442 | 29275 | 6:29928530 | 29278 | 6:30059483 | 29281 | 6:30087471 |
| 29282 | 6:30092735 | 29283 | 6:30096041 | 29284 | 6:30096126 | 29285 | 6:30100327 | 29286 | 6:30101354 |
| 29288 | 6:30105186 | 29289 | 6:30107161 | 29290 | 6:30107363 | 29291 | 6:30107470 | 29292 | 6:30107503 |
| 29293 | 6:30108091 | 29294 | 6:30108190 | 29295 | 6:30108349 | 29296 | 6:30108502 | 29297 | 6:30109134 |
| 29298 | 6:30109220 | 29299 | 6:30109285 | 29300 | 6:30109297 | 29301 | 6:30109871 | 29302 | 6:30109915 |
| 29303 | 6:30110219 | 29304 | 6:30110252 | 29305 | 6:30110638 | 29306 | 6:30111065 | 29307 | 6:30111645 |
| 29308 | 6:30112226 | 29309 | 6:30112636 | 29310 | 6:30112665 | 29311 | 6:30112688 | 29312 | 6:30112989 |
| 29313 | 6:30113025 | 29314 | 6:30113099 | 29315 | 6:30113113 | 29316 | 6:30113219 | 29317 | 6:30113503 |
| 29318 | 6:30113552 | 29319 | 6:30113737 | 29320 | 6:30114877 | 29321 | 6:30114924 | 29322 | 6:30115248 |
| 29323 | 6:30115405 | 29324 | 6:30115465 | 29325 | 6:30117653 | 29326 | 6:30117880 | 29327 | 6:30117987 |
| 29328 | 6:30118079 | 29329 | 6:30118215 | 29330 | 6:30119075 | 29331 | 6:30119163 | 29332 | 6:30119239 |
| 29333 | 6:30119492 | 29334 | 6:30120062 | 29335 | 6:30120131 | 29336 | 6:30120148 | 29337 | 6:30120317 |
| 29338 | 6:30120495 | 29339 | 6:30120714 | 29340 | 6:30120745 | 29341 | 6:30121010 | 29345 | 6:30186140 |
| 29347 | 6:30231489 | 29348 | 6:30231511 | 29349 | 6:30231899 | 29350 | 6:30231913 | 29351 | 6:30232234 |
| 29356 | 6:30429744 | 29366 | 6:30575474 | 29370 | 6:30706328 | 29373 | 6:30820768 | 29381 | 6:31067757 |
| 29386 | 6:31258805 | 29390 | 6:31380855 | 29392 | 6:31425251 | 29394 | 6:31479415 | 29397 | 6:31502542 |
| 29398 | 6:31523083 | 29409 | 6:31802402 | 29417 | 6:32011040 | 29446 | 6:33106068 | 29447 | 6:33200563 |
| 29448 | 6:33249833 | 29450 | 6:33303040 | 29454 | 6:33451849 | 29456 | 6:33555068 | 29466 | 6:33983856 |
| 29474 | 6:34086214 | 29476 | 6:34139287 | 29477 | 6:34174152 | 29478 | 6:34179094 | 29479 | 6:34185107 |
| 29480 | 6:34189894 | 29481 | 6:34197024 | 29483 | 6:34206187 | 29484 | 6:34207156 | 29485 | 6:34210015 |
| 29487 | 6:34227111 | 29488 | 6:34229172 | 29489 | 6:34236680 | 29491 | 6:34238928 | 29492 | 6:34239830 |
| 29493 | 6:34248001 | 29494 | 6:34262023 | 29495 | 6:34291379 | 29496 | 6:34292850 | 29497 | 6:34293142 |
| 29498 | 6:34297896 | 29499 | 6:34298106 | 29500 | 6:34300136 | 29501 | 6:34305846 | 29502 | 6:34313535 |
| 29503 | 6:34314736 | 29504 | 6:34316483 | 29506 | 6:34317628 | 29507 | 6:34317733 | 29508 | 6:34319685 |
| 29509 | 6:34319748 | 29510 | 6:34319965 | 29511 | 6:34320099 | 29512 | 6:34320208 | 29513 | 6:34320272 |
| 29514 | 6:34320435 | 29515 | 6:34320590 | 29516 | 6:34320597 | 29517 | 6:34321475 | 29518 | 6:34321669 |
| 29519 | 6:34321700 | 29520 | 6:34322765 | 29521 | 6:34323241 | 29522 | 6:34323355 | 29523 | 6:34323382 |
| 29524 | 6:34323399 | 29525 | 6:34323857 | 29526 | 6:34323923 | 29527 | 6:34324379 | 29528 | 6:34324509 |
| 29529 | 6:34324543 | 29530 | 6:34324794 | 29531 | 6:34324813 | 29532 | 6:34324864 | 29533 | 6:34325948 |
| 29534 | 6:34326047 | 29535 | 6:34326105 | 29536 | 6:34326672 | 29537 | 6:34326907 | 29538 | 6:34326931 |
| 29539 | 6:34327343 | 29540 | 6:34328174 | 29541 | 6:34328391 | 29542 | 6:34328595 | 29543 | 6:34328871 |
| 29544 | 6:34329287 | 29545 | 6:34329583 | 29546 | 6:34329816 | 29547 | 6:34329841 | 29548 | 6:34329959 |
| 29549 | 6:34330004 | 29550 | 6:34330105 | 29551 | 6:34330219 | 29552 | 6:34330248 | 29553 | 6:34330469 |
| 29554 | 6:34330615 | 29555 | 6:34331649 | 29556 | 6:34331871 | 29557 | 6:34334654 | 29558 | 6:34334695 |
| 29559 | 6:34334819 | 29560 | 6:34334883 | 29561 | 6:34334942 | 29562 | 6:34334975 | 29563 | 6:34335273 |
| 29564 | 6:34335676 | 29565 | 6:34336082 | 29566 | 6:34336116 | 29567 | 6:34336302 | 29568 | 6:34336518 |
| 29569 | 6:34336806 | 29570 | 6:34337142 | 29571 | 6:34337235 | 29572 | 6:34337793 | 29573 | 6:34337852 |
| 29574 | 6:34337871 | 29575 | 6:34337952 | 29576 | 6:34338244 | 29577 | 6:34338458 | 29578 | 6:34338858 |
| 29579 | 6:34338912 | 29580 | 6:34341641 | 29581 | 6:34341671 | 29582 | 6:34342381 | 29583 | 6:34342418 |
| 29584 | 6:34342439 | 29585 | 6:34342876 | 29586 | 6:34343375 | 29587 | 6:34344002 | 29588 | 6:34344707 |
| 29589 | 6:34344823 | 29590 | 6:34344911 | 29591 | 6:34345770 | 29592 | 6:34345787 | 29593 | 6:34347423 |
| 29594 | 6:34347478 | 29595 | 6:34347542 | 29596 | 6:34347870 | 29597 | 6:34347912 | 29598 | 6:34348158 |
| 29599 | 6:34348200 | 29600 | 6:34350237 | 29601 | 6:34350256 | 29602 | 6:34350284 | 29603 | 6:34352029 |
| 29604 | 6:34352540 | 29605 | 6:34353080 | 29606 | 6:34353086 | 29607 | 6:34353183 | 29608 | 6:34353582 |
| 29609 | 6:34353683 | 29610 | 6:34355309 | 29611 | 6:34356066 | 29612 | 6:34356817 | 29613 | 6:34357441 |
| 29614 | 6:34357724 | 29615 | 6:34358059 | 29616 | 6:34358159 | 29617 | 6:34359885 | 29618 | 6:34359922 |
| 29619 | 6:34360320 | 29620 | 6:34360574 | 29621 | 6:34360625 | 29622 | 6:34361019 | 29623 | 6:34361442 |
| 29624 | 6:34362625 | 29625 | 6:34363701 | 29626 | 6:34366860 | 29627 | 6:34367704 | 29628 | 6:34368646 |
| 29629 | 6:34368728 | 29630 | 6:34368895 | 29631 | 6:34369801 | 29632 | 6:34370381 | 29633 | 6:34373419 |
| 29634 | 6:34375386 | 29635 | 6:34377523 | 29636 | 6:34378377 | 29637 | 6:34378764 | 29638 | 6:34379342 |
| 29639 | 6:34379765 | 29640 | 6:34380266 | 29641 | 6:34380420 | 29642 | 6:34390859 | 29643 | 6:34398273 |
| 29644 | 6:34400215 | 29645 | 6:34401918 | 29646 | 6:34405865 | 29647 | 6:34409323 | 29648 | 6:34412354 |
| 29649 | 6:34412446 | 29650 | 6:34417593 | 29651 | 6:34423276 | 29652 | 6:34425931 | 29653 | 6:34429298 |
| 29654 | 6:34433083 | 29655 | 6:34436717 | 29656 | 6:34443045 | 29657 | 6:34445489 | 29658 | 6:34454602 |
| 29661 | 6:34485191 | 29663 | 6:34508971 | 29664 | 6:34511326 | 29665 | 6:34512053 | 29666 | 6:34512149 |
| 29667 | 6:34512224 | 29668 | 6:34512436 | 29669 | 6:34512607 | 29670 | 6:34514009 | 29671 | 6:34520073 |
| 29672 | 6:34520205 | 29673 | 6:34520340 | 29674 | 6:34521287 | 29675 | 6:34521294 | 29676 | 6:34522877 |
| 29677 | 6:34523485 | 29678 | 6:34524720 | 29679 | 6:34526737 | 29680 | 6:34527170 | 29681 | 6:34531081 |
| 29682 | 6:34531219 | 29683 | 6:34531228 | 29684 | 6:34532115 | 29685 | 6:34532276 | 29686 | 6:34533970 |
| 29687 | 6:34534131 | 29688 | 6:34534507 | 29689 | 6:34534849 | 29690 | 6:34534873 | 29691 | 6:34535922 |
| 29692 | 6:34535958 | 29693 | 6:34535980 | 29694 | 6:34537513 | 29695 | 6:34538518 | 29696 | 6:34538586 |
| 29697 | 6:34538798 | 29698 | 6:34539189 | 29699 | 6:34544253 | 29700 | 6:34544296 | 29701 | 6:34544650 |
| 29702 | 6:34544882 | 29703 | 6:34544924 | 29704 | 6:34545143 | 29705 | 6:34545442 | 29706 | 6:34545976 |
| 29707 | 6:34546002 | 29708 | 6:34546098 | 29709 | 6:34546230 | 29710 | 6:34546882 | 29711 | 6:34551061 |
| 29712 | 6:34551087 | 29713 | 6:34551357 | 29714 | 6:34551821 | 29715 | 6:34552091 | 29716 | 6:34552302 |
| 29717 | 6:34559140 | 29719 | 6:34568139 | 29720 | 6:34570377 | 29721 | 6:34571403 | 29723 | 6:34585218 |
| 29724 | 6:34587383 | 29726 | 6:34590054 | 29727 | 6:34590568 | 29728 | 6:34590836 | 29729 | 6:34590891 |
| 29730 | 6:34594553 | 29731 | 6:34595913 | 29732 | 6:34605484 | 29734 | 6:34606444 | 29735 | 6:34606508 |
| 29736 | 6:34607125 | 29737 | 6:34607239 | 29738 | 6:34608122 | 29739 | 6:34608604 | 29740 | 6:34609191 |
| 29741 | 6:34609820 | 29742 | 6:34609854 | 29743 | 6:34610451 | 29744 | 6:34610729 | 29745 | 6:34610740 |
| 29746 | 6:34610765 | 29748 | 6:34610849 | 29749 | 6:34611386 | 29750 | 6:34611923 | 29751 | 6:34611955 |
| 29752 | 6:34612600 | 29753 | 6:34613447 | 29754 | 6:34614887 | 29755 | 6:34615063 | 29756 | 6:34617013 |
| 29757 | 6:34617195 | 29758 | 6:34617208 | 29759 | 6:34617293 | 29760 | 6:34617674 | 29761 | 6:34617812 |
| 29762 | 6:34617865 | 29763 | 6:34617943 | 29764 | 6:34618113 | 29765 | 6:34618226 | 29766 | 6:34618258 |
| 29767 | 6:34618461 | 29768 | 6:34618467 | 29769 | 6:34618532 | 29770 | 6:34618622 | 29771 | 6:34618679 |
| 29772 | 6:34618784 | 29773 | 6:34618813 | 29774 | 6:34618880 | 29775 | 6:34620004 | 29776 | 6:34620108 |
| 29777 | 6:34620118 | 29778 | 6:34620548 | 29779 | 6:34621825 | 29780 | 6:34622374 | 29781 | 6:34623163 |
| 29782 | 6:34623346 | 29783 | 6:34623681 | 29784 | 6:34623706 | 29785 | 6:34623745 | 29786 | 6:34624441 |
| 29787 | 6:34629181 | 29788 | 6:34629567 | 29789 | 6:34629581 | 29790 | 6:34629692 | 29791 | 6:34629900 |
| 29792 | 6:34629975 | 29793 | 6:34629990 | 29794 | 6:34630073 | 29795 | 6:34630347 | 29796 | 6:34630587 |
| 29797 | 6:34630602 | 29798 | 6:34630922 | 29799 | 6:34631246 | 29800 | 6:34631446 | 29801 | 6:34631808 |
| 29802 | 6:34631843 | 29803 | 6:34632098 | 29804 | 6:34632375 | 29805 | 6:34632405 | 29806 | 6:34632714 |
| 29807 | 6:34632833 | 29808 | 6:34633050 | 29809 | 6:34633159 | 29810 | 6:34633187 | 29811 | 6:34633229 |
| 29812 | 6:34633562 | 29813 | 6:34633782 | 29814 | 6:34633841 | 29815 | 6:34633967 | 29816 | 6:34633990 |
| 29817 | 6:34634055 | 29818 | 6:34634169 | 29819 | 6:34634190 | 29820 | 6:34634250 | 29821 | 6:34634312 |
| 29822 | 6:34634801 | 29823 | 6:34634990 | 29824 | 6:34635003 | 29825 | 6:34635530 | 29826 | 6:34635703 |
| 29827 | 6:34636131 | 29828 | 6:34636138 | 29829 | 6:34636209 | 29830 | 6:34637085 | 29831 | 6:34640553 |
| 29832 | 6:34641695 | 29833 | 6:34647407 | 29834 | 6:34657657 | 29835 | 6:34661296 | 29836 | 6:34661323 |
| 29837 | 6:34662849 | 29838 | 6:34665463 | 29839 | 6:34668000 | 29840 | 6:34670574 | 29841 | 6:34671333 |
| 29842 | 6:34673238 | 29844 | 6:34686618 | 29845 | 6:34687343 | 29846 | 6:34689725 | 29847 | 6:34697260 |
| 29848 | 6:34699079 | 29849 | 6:34703681 | 29850 | 6:34704601 | 29851 | 6:34704806 | 29852 | 6:34705455 |
| 29853 | 6:34714302 | 29854 | 6:34714956 | 29855 | 6:34724151 | 29856 | 6:34726853 | 29857 | 6:34727995 |
| 29858 | 6:34728449 | 29859 | 6:34730148 | 29860 | 6:34730862 | 29861 | 6:34734530 | 29863 | 6:34738528 |
| 29864 | 6:34746517 | 29865 | 6:34751152 | 29866 | 6:34751196 | 29867 | 6:34751889 | 29868 | 6:34753115 |
| 29869 | 6:34762474 | 29870 | 6:34762792 | 29871 | 6:34762888 | 29872 | 6:34766154 | 29873 | 6:34766590 |
| 29874 | 6:34766756 | 29875 | 6:34768280 | 29876 | 6:34768301 | 29877 | 6:34768997 | 29878 | 6:34769036 |
| 29879 | 6:34769720 | 29880 | 6:34770109 | 29881 | 6:34770808 | 29882 | 6:34777728 | 29883 | 6:34780553 |
| 29884 | 6:34781550 | 29885 | 6:34781934 | 29886 | 6:34782739 | 29887 | 6:34782759 | 29888 | 6:34783042 |
| 29889 | 6:34783961 | 29890 | 6:34784243 | 29891 | 6:34784299 | 29892 | 6:34784335 | 29893 | 6:34784977 |
| 29894 | 6:34785811 | 29895 | 6:34785864 | 29896 | 6:34785916 | 29897 | 6:34786001 | 29898 | 6:34786006 |
| 29899 | 6:34786198 | 29900 | 6:34786203 | 29901 | 6:34786474 | 29902 | 6:34786586 | 29903 | 6:34787240 |
| 29904 | 6:34789109 | 29905 | 6:34792867 | 29907 | 6:34845861 | 29909 | 6:34929550 | 29910 | 6:34932019 |
| 29911 | 6:34933373 | 29912 | 6:34943666 | 29913 | 6:34961634 | 29915 | 6:34972640 | 29916 | 6:34976990 |
| 29917 | 6:34985119 | 29918 | 6:34990679 | 29919 | 6:34992674 | 29920 | 6:34994097 | 29921 | 6:34995220 |
| 29922 | 6:35021191 | 29923 | 6:35047210 | 29924 | 6:35140608 | 29927 | 6:35157410 | 29929 | 6:35160353 |
| 29930 | 6:35161520 | 29932 | 6:35162419 | 29934 | 6:35163337 | 29935 | 6:35165014 | 29936 | 6:35166488 |
| 29937 | 6:35166725 | 29942 | 6:35245781 | 29945 | 6:35274129 | 29948 | 6:35381936 | 29953 | 6:35504728 |
| 29957 | 6:35561016 | 29958 | 6:35561546 | 29959 | 6:35561595 | 29960 | 6:35581628 | 29961 | 6:35582162 |
| 29962 | 6:35582223 | 29963 | 6:35587094 | 29964 | 6:35594486 | 29965 | 6:35609063 | 29966 | 6:35616265 |
| 29967 | 6:35616280 | 29969 | 6:35618707 | 29974 | 6:35666246 | 29975 | 6:35678704 | 29976 | 6:35678754 |
| 29977 | 6:35684254 | 29978 | 6:35703001 | 29979 | 6:35713846 | 29980 | 6:35718341 | 29981 | 6:35726574 |
| 29982 | 6:35732398 | 29984 | 6:35792391 | 29985 | 6:35825879 | 29987 | 6:35886281 | 29989 | 6:35897314 |
| 29994 | 6:35908980 | 30001 | 6:35937425 | 30002 | 6:35937819 | 30003 | 6:35937874 | 30004 | 6:35941557 |
| 30006 | 6:35948457 | 30007 | 6:35963118 | 30008 | 6:35968631 | 30009 | 6:35972831 | 30012 | 6:35992028 |
| 30014 | 6:36011027 | 30015 | 6:36011772 | 30018 | 6:36032905 | 30019 | 6:36034678 | 30020 | 6:36036595 |
| 30029 | 6:36125455 | 30030 | 6:36143409 | 30031 | 6:36145256 | 30032 | 6:36147418 | 30033 | 6:36148375 |
| 30034 | 6:36149041 | 30035 | 6:36150876 | 30036 | 6:36151325 | 30037 | 6:36153013 | 30038 | 6:36156606 |
| 30039 | 6:36156894 | 30040 | 6:36157622 | 30041 | 6:36157988 | 30042 | 6:36158361 | 30043 | 6:36158500 |
| 30044 | 6:36159608 | 30045 | 6:36161275 | 30046 | 6:36161377 | 30047 | 6:36161627 | 30048 | 6:36161635 |
| 30049 | 6:36162952 | 30050 | 6:36163342 | 30051 | 6:36163430 | 30052 | 6:36164022 | 30053 | 6:36164566 |
| 30054 | 6:36165003 | 30055 | 6:36165139 | 30056 | 6:36165152 | 30057 | 6:36165220 | 30058 | 6:36165307 |
| 30059 | 6:36166110 | 30060 | 6:36166180 | 30061 | 6:36166207 | 30062 | 6:36166505 | 30063 | 6:36166793 |
| 30064 | 6:36166797 | 30065 | 6:36167327 | 30066 | 6:36167830 | 30067 | 6:36168126 | 30068 | 6:36168722 |
| 30069 | 6:36169714 | 30070 | 6:36169794 | 30071 | 6:36170135 | 30072 | 6:36170201 | 30073 | 6:36171686 |
| 30074 | 6:36172372 | 30075 | 6:36177201 | 30076 | 6:36180645 | 30077 | 6:36181021 | 30078 | 6:36182794 |
| 30079 | 6:36183913 | 30080 | 6:36186583 | 30081 | 6:36191256 | 30082 | 6:36198702 | 30083 | 6:36204036 |
| 30084 | 6:36211597 | 30085 | 6:36212186 | 30086 | 6:36219844 | 30087 | 6:36225618 | 30088 | 6:36236546 |
| 30089 | 6:36238601 | 30090 | 6:36238793 | 30091 | 6:36239080 | 30092 | 6:36239773 | 30093 | 6:36240264 |
| 30094 | 6:36241246 | 30095 | 6:36246325 | 30096 | 6:36248262 | 30097 | 6:36249143 | 30098 | 6:36250084 |
| 30099 | 6:36254264 | 30100 | 6:36255334 | 30101 | 6:36256227 | 30102 | 6:36256264 | 30103 | 6:36256316 |
| 30104 | 6:36261278 | 30105 | 6:36262429 | 30106 | 6:36263719 | 30107 | 6:36264671 | 30108 | 6:36264792 |
| 30109 | 6:36265379 | 30110 | 6:36272248 | 30111 | 6:36273670 | 30112 | 6:36277826 | 30113 | 6:36280253 |
| 30114 | 6:36283110 | 30115 | 6:36283198 | 30116 | 6:36286200 | 30117 | 6:36292811 | 30118 | 6:36298957 |
| 30119 | 6:36299463 | 30120 | 6:36299867 | 30121 | 6:36300007 | 30122 | 6:36324361 | 30123 | 6:36333247 |
| 30124 | 6:36336622 | 30125 | 6:36336765 | 30126 | 6:36337366 | 30127 | 6:36340265 | 30128 | 6:36363919 |
| 30129 | 6:36365275 | 30131 | 6:36412092 | 30141 | 6:36436269 | 30142 | 6:36440409 | 30143 | 6:36441375 |
| 30144 | 6:36443819 | 30146 | 6:36445226 | 30149 | 6:36454421 | 30151 | 6:36457471 | 30152 | 6:36457509 |
| 30153 | 6:36458519 | 30154 | 6:36460784 | 30155 | 6:36461234 | 30156 | 6:36461574 | 30157 | 6:36463931 |
| 30158 | 6:36466353 | 30159 | 6:36466871 | 30161 | 6:36469381 | 30162 | 6:36469975 | 30163 | 6:36474704 |
| 30166 | 6:36478628 | 30167 | 6:36480305 | 30170 | 6:36482113 | 30172 | 6:36487754 | 30173 | 6:36489160 |
| 30176 | 6:36491203 | 30177 | 6:36491990 | 30178 | 6:36499104 | 30179 | 6:36499659 | 30180 | 6:36500389 |
| 30181 | 6:36501474 | 30182 | 6:36503509 | 30183 | 6:36506439 | 30184 | 6:36506901 | 30185 | 6:36509073 |
| 30186 | 6:36514666 | 30187 | 6:36514687 | 30188 | 6:36516071 | 30189 | 6:36518117 | 30190 | 6:36524445 |
| 30191 | 6:36524489 | 30192 | 6:36529972 | 30193 | 6:36530565 | 30194 | 6:36531251 | 30195 | 6:36531630 |
| 30197 | 6:36532751 | 30198 | 6:36533529 | 30199 | 6:36533756 | 30200 | 6:36533947 | 30201 | 6:36534891 |
| 30202 | 6:36535012 | 30203 | 6:36535227 | 30204 | 6:36535356 | 30205 | 6:36535863 | 30206 | 6:36535904 |
| 30207 | 6:36536565 | 30208 | 6:36537055 | 30209 | 6:36537210 | 30210 | 6:36537223 | 30211 | 6:36537284 |
| 30212 | 6:36537515 | 30213 | 6:36537657 | 30214 | 6:36537708 | 30215 | 6:36538044 | 30216 | 6:36538107 |
| 30217 | 6:36538113 | 30218 | 6:36538805 | 30219 | 6:36538827 | 30220 | 6:36539051 | 30221 | 6:36539508 |
| 30222 | 6:36539630 | 30223 | 6:36540193 | 30224 | 6:36540734 | 30225 | 6:36540815 | 30226 | 6:36543846 |
| 30227 | 6:36544549 | 30228 | 6:36544681 | 30229 | 6:36544687 | 30230 | 6:36544786 | 30231 | 6:36544914 |
| 30232 | 6:36545202 | 30233 | 6:36545304 | 30235 | 6:36547031 | 30236 | 6:36547078 | 30237 | 6:36547888 |
| 30238 | 6:36547972 | 30239 | 6:36548012 | 30240 | 6:36548205 | 30241 | 6:36548953 | 30242 | 6:36549261 |
| 30243 | 6:36549683 | 30244 | 6:36550069 | 30245 | 6:36550199 | 30246 | 6:36551621 | 30247 | 6:36552037 |
| 30248 | 6:36552742 | 30249 | 6:36552915 | 30252 | 6:36556271 | 30253 | 6:36556442 | 30254 | 6:36557671 |
| 30256 | 6:36561952 | 30258 | 6:36567616 | 30262 | 6:36569777 | 30263 | 6:36571430 | 30265 | 6:36594692 |
| 30266 | 6:36598347 | 30267 | 6:36599192 | 30268 | 6:36602971 | 30269 | 6:36603958 | 30270 | 6:36608521 |
| 30271 | 6:36608642 | 30272 | 6:36611873 | 30273 | 6:36612067 | 30274 | 6:36614641 | 30275 | 6:36617150 |
| 30276 | 6:36618712 | 30277 | 6:36619708 | 30278 | 6:36620654 | 30279 | 6:36621895 | 30280 | 6:36622608 |
| 30281 | 6:36624334 | 30282 | 6:36625203 | 30283 | 6:36625485 | 30285 | 6:36626119 | 30286 | 6:36626509 |
| 30287 | 6:36629387 | 30288 | 6:36631261 | 30289 | 6:36632276 | 30290 | 6:36632779 | 30291 | 6:36633630 |
| 30292 | 6:36633929 | 30293 | 6:36634989 | 30294 | 6:36635404 | 30295 | 6:36635605 | 30296 | 6:36635939 |
| 30297 | 6:36638521 | 30298 | 6:36638963 | 30299 | 6:36639106 | 30300 | 6:36642083 | 30301 | 6:36642499 |
| 30302 | 6:36643100 | 30303 | 6:36643378 | 30304 | 6:36644151 | 30305 | 6:36645330 | 30306 | 6:36646194 |
| 30307 | 6:36646596 | 30308 | 6:36648068 | 30309 | 6:36648160 | 30310 | 6:36653563 | 30311 | 6:36657483 |
| 30312 | 6:36664663 | 30313 | 6:36673216 | 30314 | 6:36679692 | 30316 | 6:36681772 | 30318 | 6:36695293 |
| 30319 | 6:36700131 | 30320 | 6:36700135 | 30321 | 6:36706370 | 30322 | 6:36720331 | 30324 | 6:36743854 |
| 30325 | 6:36773960 | 30326 | 6:36787210 | 30331 | 6:36812951 | 30332 | 6:36813102 | 30333 | 6:36813124 |
| 30334 | 6:36813128 | 30335 | 6:36813593 | 30336 | 6:36813633 | 30337 | 6:36814581 | 30338 | 6:36814786 |
| 30339 | 6:36815144 | 30340 | 6:36815200 | 30341 | 6:36815490 | 30342 | 6:36815725 | 30343 | 6:36815817 |
| 30344 | 6:36816067 | 30345 | 6:36816314 | 30346 | 6:36816419 | 30347 | 6:36817159 | 30348 | 6:36817266 |
| 30349 | 6:36817581 | 30350 | 6:36817596 | 30351 | 6:36817683 | 30352 | 6:36817713 | 30353 | 6:36817969 |
| 30354 | 6:36818237 | 30355 | 6:36818704 | 30356 | 6:36818808 | 30357 | 6:36819594 | 30358 | 6:36819695 |
| 30359 | 6:36819751 | 30360 | 6:36820074 | 30361 | 6:36820138 | 30362 | 6:36820361 | 30363 | 6:36820770 |
| 30364 | 6:36821432 | 30365 | 6:36821474 | 30366 | 6:36822273 | 30367 | 6:36822526 | 30368 | 6:36822568 |
| 30369 | 6:36822744 | 30370 | 6:36823102 | 30371 | 6:36823606 | 30372 | 6:36825509 | 30373 | 6:36830103 |
| 30374 | 6:36830934 | 30375 | 6:36831012 | 30376 | 6:36831131 | 30377 | 6:36831274 | 30378 | 6:36831813 |
| 30379 | 6:36831871 | 30380 | 6:36834692 | 30381 | 6:36835108 | 30391 | 6:36949956 | 30392 | 6:36952579 |
| 30393 | 6:36954127 | 30395 | 6:36959305 | 30396 | 6:36960146 | 30397 | 6:36970451 | 30398 | 6:36978207 |
| 30399 | 6:36978216 | 30400 | 6:36978361 | 30401 | 6:36978371 | 30402 | 6:36978422 | 30403 | 6:36978431 |
| 30404 | 6:36978698 | 30405 | 6:36978703 | 30406 | 6:36978958 | 30407 | 6:36979025 | 30408 | 6:36979081 |
| 30409 | 6:36979093 | 30410 | 6:36979116 | 30411 | 6:36979134 | 30412 | 6:36979146 | 30413 | 6:36981664 |
| 30414 | 6:36981815 | 30415 | 6:36985783 | 30416 | 6:36985804 | 30417 | 6:36995904 | 30419 | 6:37010986 |
| 30420 | 6:37011036 | 30421 | 6:37016748 | 30422 | 6:37017628 | 30423 | 6:37017833 | 30424 | 6:37018373 |
| 30425 | 6:37018495 | 30426 | 6:37018672 | 30427 | 6:37018745 | 30428 | 6:37018878 | 30429 | 6:37019035 |
| 30430 | 6:37019263 | 30431 | 6:37019274 | 30432 | 6:37019373 | 30433 | 6:37019415 | 30434 | 6:37019464 |
| 30435 | 6:37019512 | 30436 | 6:37019519 | 30437 | 6:37019606 | 30438 | 6:37020100 | 30439 | 6:37020244 |
| 30440 | 6:37020255 | 30441 | 6:37020456 | 30442 | 6:37020574 | 30443 | 6:37020591 | 30444 | 6:37020608 |
| 30445 | 6:37021123 | 30446 | 6:37021154 | 30447 | 6:37021520 | 30448 | 6:37021604 | 30449 | 6:37021714 |
| 30450 | 6:37021968 | 30451 | 6:37022137 | 30452 | 6:37022181 | 30453 | 6:37022238 | 30454 | 6:37023645 |
| 30460 | 6:37030480 | 30461 | 6:37032906 | 30463 | 6:37034096 | 30464 | 6:37034286 | 30465 | 6:37034823 |
| 30466 | 6:37034912 | 30467 | 6:37035186 | 30469 | 6:37038017 | 30470 | 6:37044068 | 30473 | 6:37061527 |
| 30474 | 6:37074563 | 30475 | 6:37075910 | 30476 | 6:37078470 | 30477 | 6:37078665 | 30478 | 6:37078781 |
| 30479 | 6:37079090 | 30480 | 6:37079147 | 30481 | 6:37079324 | 30482 | 6:37079422 | 30483 | 6:37079559 |
| 30484 | 6:37079653 | 30485 | 6:37079666 | 30486 | 6:37080062 | 30487 | 6:37080097 | 30488 | 6:37080210 |
| 30489 | 6:37080486 | 30490 | 6:37080919 | 30491 | 6:37080936 | 30492 | 6:37081034 | 30493 | 6:37081117 |
| 30494 | 6:37081212 | 30495 | 6:37081319 | 30496 | 6:37081745 | 30497 | 6:37081947 | 30498 | 6:37082116 |
| 30499 | 6:37082537 | 30500 | 6:37082542 | 30501 | 6:37082592 | 30502 | 6:37082641 | 30503 | 6:37082764 |
| 30504 | 6:37082830 | 30505 | 6:37083023 | 30506 | 6:37083086 | 30507 | 6:37083196 | 30508 | 6:37083881 |
| 30509 | 6:37084518 | 30510 | 6:37084543 | 30511 | 6:37084979 | 30512 | 6:37084987 | 30513 | 6:37084991 |
| 30514 | 6:37085423 | 30515 | 6:37085516 | 30516 | 6:37087274 | 30517 | 6:37087642 | 30518 | 6:37087987 |
| 30519 | 6:37087999 | 30520 | 6:37088359 | 30521 | 6:37088506 | 30522 | 6:37088510 | 30523 | 6:37088831 |
| 30524 | 6:37088835 | 30525 | 6:37088955 | 30526 | 6:37089045 | 30527 | 6:37089108 | 30528 | 6:37089125 |
| 30529 | 6:37089202 | 30530 | 6:37089209 | 30531 | 6:37089234 | 30532 | 6:37089651 | 30533 | 6:37089673 |
| 30534 | 6:37089763 | 30535 | 6:37089819 | 30536 | 6:37089854 | 30537 | 6:37089926 | 30538 | 6:37090001 |
| 30539 | 6:37090127 | 30540 | 6:37090147 | 30541 | 6:37090190 | 30542 | 6:37090229 | 30543 | 6:37090257 |
| 30544 | 6:37090343 | 30545 | 6:37090838 | 30546 | 6:37090865 | 30547 | 6:37091359 | 30548 | 6:37091365 |
| 30549 | 6:37091616 | 30550 | 6:37091767 | 30551 | 6:37092094 | 30552 | 6:37092116 | 30553 | 6:37092227 |
| 30554 | 6:37092573 | 30555 | 6:37092869 | 30556 | 6:37092904 | 30557 | 6:37092936 | 30558 | 6:37093073 |
| 30559 | 6:37093934 | 30560 | 6:37093949 | 30561 | 6:37094212 | 30562 | 6:37094603 | 30563 | 6:37094693 |
| 30564 | 6:37094789 | 30565 | 6:37094827 | 30566 | 6:37095299 | 30567 | 6:37095517 | 30568 | 6:37096027 |
| 30569 | 6:37096074 | 30570 | 6:37096486 | 30571 | 6:37096495 | 30572 | 6:37096649 | 30573 | 6:37096879 |
| 30574 | 6:37096959 | 30575 | 6:37097509 | 30576 | 6:37097522 | 30577 | 6:37097635 | 30578 | 6:37097772 |
| 30579 | 6:37097995 | 30580 | 6:37098052 | 30581 | 6:37098220 | 30582 | 6:37098309 | 30583 | 6:37098640 |
| 30584 | 6:37098716 | 30585 | 6:37098782 | 30586 | 6:37098831 | 30587 | 6:37098872 | 30588 | 6:37099284 |
| 30589 | 6:37099535 | 30590 | 6:37099560 | 30591 | 6:37099942 | 30592 | 6:37099978 | 30593 | 6:37099983 |
| 30594 | 6:37100199 | 30595 | 6:37100203 | 30596 | 6:37100223 | 30597 | 6:37100242 | 30598 | 6:37100749 |
| 30599 | 6:37100852 | 30600 | 6:37100906 | 30601 | 6:37100924 | 30602 | 6:37101114 | 30603 | 6:37101213 |
| 30604 | 6:37101414 | 30605 | 6:37101664 | 30606 | 6:37101691 | 30607 | 6:37104751 | 30608 | 6:37104774 |
| 30609 | 6:37104851 | 30610 | 6:37104926 | 30611 | 6:37104993 | 30612 | 6:37105044 | 30613 | 6:37105252 |
| 30614 | 6:37105257 | 30615 | 6:37106212 | 30616 | 6:37107589 | 30617 | 6:37107774 | 30618 | 6:37108028 |
| 30619 | 6:37108237 | 30620 | 6:37112827 | 30621 | 6:37112864 | 30622 | 6:37116160 | 30623 | 6:37116221 |
| 30624 | 6:37116481 | 30625 | 6:37116730 | 30626 | 6:37117771 | 30627 | 6:37117988 | 30628 | 6:37120342 |
| 30630 | 6:37123023 | 30631 | 6:37128820 | 30634 | 6:37136040 | 30635 | 6:37136706 | 30636 | 6:37136794 |
| 30637 | 6:37138117 | 30638 | 6:37138139 | 30639 | 6:37138262 | 30640 | 6:37138535 | 30641 | 6:37139425 |
| 30642 | 6:37140287 | 30643 | 6:37142174 | 30644 | 6:37142953 | 30645 | 6:37144122 | 30646 | 6:37147544 |
| 30647 | 6:37147627 | 30648 | 6:37149236 | 30649 | 6:37149945 | 30650 | 6:37150579 | 30651 | 6:37151586 |
| 30652 | 6:37151618 | 30654 | 6:37152332 | 30656 | 6:37153197 | 30657 | 6:37153310 | 30658 | 6:37153398 |
| 30659 | 6:37153558 | 30660 | 6:37153831 | 30661 | 6:37155597 | 30662 | 6:37156064 | 30663 | 6:37156337 |
| 30664 | 6:37156677 | 30665 | 6:37156889 | 30666 | 6:37157333 | 30667 | 6:37157660 | 30668 | 6:37158136 |
| 30669 | 6:37158289 | 30670 | 6:37158384 | 30671 | 6:37159298 | 30672 | 6:37159341 | 30673 | 6:37159410 |
| 30674 | 6:37159427 | 30675 | 6:37159591 | 30676 | 6:37159718 | 30677 | 6:37159778 | 30678 | 6:37159892 |
| 30679 | 6:37160005 | 30680 | 6:37160134 | 30681 | 6:37160139 | 30682 | 6:37160559 | 30683 | 6:37161039 |
| 30684 | 6:37161146 | 30685 | 6:37161408 | 30686 | 6:37161619 | 30687 | 6:37161712 | 30688 | 6:37162703 |
| 30689 | 6:37162813 | 30690 | 6:37162822 | 30691 | 6:37163101 | 30692 | 6:37163223 | 30693 | 6:37163776 |
| 30694 | 6:37163934 | 30695 | 6:37164094 | 30696 | 6:37164218 | 30697 | 6:37164439 | 30698 | 6:37164529 |
| 30699 | 6:37164825 | 30700 | 6:37165013 | 30701 | 6:37165306 | 30702 | 6:37165536 | 30703 | 6:37165545 |
| 30704 | 6:37165588 | 30705 | 6:37165831 | 30706 | 6:37165836 | 30707 | 6:37166157 | 30708 | 6:37166449 |
| 30709 | 6:37166482 | 30710 | 6:37167166 | 30711 | 6:37167349 | 30712 | 6:37167472 | 30713 | 6:37167487 |
| 30714 | 6:37167778 | 30715 | 6:37167868 | 30716 | 6:37167910 | 30717 | 6:37167946 | 30718 | 6:37168208 |
| 30719 | 6:37168379 | 30720 | 6:37168414 | 30721 | 6:37168500 | 30722 | 6:37168914 | 30723 | 6:37168959 |
| 30724 | 6:37169024 | 30725 | 6:37169066 | 30726 | 6:37169247 | 30727 | 6:37169728 | 30728 | 6:37169746 |
| 30729 | 6:37169824 | 30730 | 6:37171398 | 30731 | 6:37171831 | 30732 | 6:37172063 | 30733 | 6:37172194 |
| 30734 | 6:37172300 | 30736 | 6:37173048 | 30737 | 6:37173169 | 30738 | 6:37173382 | 30739 | 6:37174433 |
| 30740 | 6:37174739 | 30741 | 6:37174812 | 30742 | 6:37174838 | 30743 | 6:37174847 | 30744 | 6:37174944 |
| 30745 | 6:37175216 | 30746 | 6:37175405 | 30747 | 6:37175422 | 30749 | 6:37175709 | 30751 | 6:37175915 |
| 30752 | 6:37175930 | 30753 | 6:37175957 | 30754 | 6:37176060 | 30755 | 6:37176229 | 30756 | 6:37176528 |
| 30758 | 6:37176686 | 30759 | 6:37177441 | 30760 | 6:37177466 | 30761 | 6:37177638 | 30762 | 6:37177788 |
| 30763 | 6:37177792 | 30764 | 6:37177809 | 30765 | 6:37178122 | 30766 | 6:37178336 | 30767 | 6:37178722 |
| 30768 | 6:37179535 | 30769 | 6:37180932 | 30770 | 6:37180966 | 30771 | 6:37181059 | 30772 | 6:37181087 |
| 30773 | 6:37181206 | 30774 | 6:37181374 | 30775 | 6:37181533 | 30776 | 6:37181894 | 30777 | 6:37182068 |
| 30778 | 6:37182373 | 30779 | 6:37182427 | 30780 | 6:37182621 | 30782 | 6:37182810 | 30783 | 6:37182983 |
| 30784 | 6:37183090 | 30785 | 6:37183609 | 30786 | 6:37183649 | 30789 | 6:37185316 | 30790 | 6:37188198 |
| 30793 | 6:37191616 | 30795 | 6:37193241 | 30796 | 6:37195555 | 30800 | 6:37211057 | 30801 | 6:37214068 |
| 30802 | 6:37214389 | 30803 | 6:37214736 | 30804 | 6:37216084 | 30805 | 6:37216293 | 30806 | 6:37217600 |
| 30807 | 6:37218354 | 30808 | 6:37218423 | 30809 | 6:37219372 | 30811 | 6:37221337 | 30812 | 6:37221507 |
| 30813 | 6:37221776 | 30814 | 6:37222424 | 30815 | 6:37225729 | 30816 | 6:37226016 | 30817 | 6:37226725 |
| 30819 | 6:37228661 | 30821 | 6:37231769 | 30822 | 6:37232090 | 30823 | 6:37232142 | 30824 | 6:37232577 |
| 30825 | 6:37233429 | 30826 | 6:37233659 | 30827 | 6:37234042 | 30828 | 6:37234954 | 30831 | 6:37236226 |
| 30832 | 6:37237969 | 30833 | 6:37238128 | 30834 | 6:37238402 | 30835 | 6:37238486 | 30836 | 6:37238502 |
| 30837 | 6:37238713 | 30838 | 6:37238946 | 30839 | 6:37241792 | 30840 | 6:37241812 | 30841 | 6:37243159 |
| 30842 | 6:37243373 | 30843 | 6:37243677 | 30845 | 6:37244804 | 30846 | 6:37245026 | 30847 | 6:37245138 |
| 30849 | 6:37247874 | 30850 | 6:37248754 | 30853 | 6:37251182 | 30854 | 6:37252978 | 30855 | 6:37255945 |
| 30860 | 6:37263319 | 30861 | 6:37264863 | 30862 | 6:37265148 | 30863 | 6:37266735 | 30866 | 6:37270930 |
| 30868 | 6:37273774 | 30869 | 6:37278799 | 30870 | 6:37281308 | 30871 | 6:37281335 | 30872 | 6:37282356 |
| 30873 | 6:37284874 | 30874 | 6:37285174 | 30875 | 6:37285822 | 30876 | 6:37285843 | 30877 | 6:37286325 |
| 30878 | 6:37286380 | 30880 | 6:37286722 | 30889 | 6:37289572 | 30904 | 6:37293699 | 30906 | 6:37294860 |
| 30907 | 6:37295049 | 30908 | 6:37295154 | 30909 | 6:37295245 | 30910 | 6:37295403 | 30911 | 6:37295803 |
| 30912 | 6:37296114 | 30913 | 6:37297426 | 30914 | 6:37297697 | 30915 | 6:37298251 | 30916 | 6:37298617 |
| 30917 | 6:37298725 | 30918 | 6:37298786 | 30919 | 6:37299349 | 30920 | 6:37299587 | 30921 | 6:37299855 |
| 30922 | 6:37299968 | 30923 | 6:37300447 | 30924 | 6:37300499 | 30925 | 6:37300590 | 30926 | 6:37300767 |
| 30927 | 6:37300810 | 30928 | 6:37300872 | 30929 | 6:37301720 | 30930 | 6:37301762 | 30931 | 6:37301893 |
| 30932 | 6:37301959 | 30933 | 6:37302011 | 30934 | 6:37302172 | 30935 | 6:37302478 | 30936 | 6:37302499 |
| 30937 | 6:37302730 | 30938 | 6:37303255 | 30939 | 6:37303264 | 30940 | 6:37303500 | 30941 | 6:37303542 |
| 30942 | 6:37303727 | 30943 | 6:37304109 | 30944 | 6:37304324 | 30945 | 6:37304493 | 30947 | 6:37304564 |
| 30948 | 6:37304601 | 30949 | 6:37304640 | 30950 | 6:37304825 | 30951 | 6:37305062 | 30952 | 6:37305466 |
| 30953 | 6:37305470 | 30954 | 6:37305490 | 30955 | 6:37305557 | 30956 | 6:37306944 | 30957 | 6:37306975 |
| 30958 | 6:37307238 | 30961 | 6:37310693 | 30962 | 6:37311375 | 30963 | 6:37311766 | 30964 | 6:37311816 |
| 30965 | 6:37311980 | 30966 | 6:37312071 | 30967 | 6:37312238 | 30968 | 6:37312270 | 30969 | 6:37312326 |
| 30970 | 6:37312692 | 30971 | 6:37313215 | 30972 | 6:37313503 | 30973 | 6:37313663 | 30974 | 6:37313742 |
| 30975 | 6:37313954 | 30976 | 6:37314022 | 30977 | 6:37314183 | 30978 | 6:37314291 | 30979 | 6:37315223 |
| 30980 | 6:37315480 | 30981 | 6:37315752 | 30982 | 6:37315774 | 30983 | 6:37316117 | 30984 | 6:37316265 |
| 30985 | 6:37316471 | 30986 | 6:37316594 | 30987 | 6:37316659 | 30988 | 6:37316928 | 30989 | 6:37317087 |
| 30990 | 6:37317153 | 30991 | 6:37317167 | 30992 | 6:37317341 | 30993 | 6:37317695 | 30994 | 6:37318285 |
| 30995 | 6:37318305 | 30996 | 6:37318658 | 30997 | 6:37318743 | 30998 | 6:37318847 | 30999 | 6:37319284 |
| 31000 | 6:37319902 | 31001 | 6:37320093 | 31002 | 6:37321012 | 31003 | 6:37321049 | 31004 | 6:37321441 |
| 31005 | 6:37321485 | 31006 | 6:37322361 | 31007 | 6:37322770 | 31008 | 6:37323838 | 31009 | 6:37323843 |
| 31010 | 6:37324718 | 31011 | 6:37325097 | 31012 | 6:37325189 | 31013 | 6:37326965 | 31014 | 6:37327393 |
| 31015 | 6:37327644 | 31016 | 6:37328014 | 31017 | 6:37328069 | 31018 | 6:37328528 | 31019 | 6:37328898 |
| 31020 | 6:37329042 | 31021 | 6:37329061 | 31022 | 6:37329319 | 31023 | 6:37329413 | 31024 | 6:37329526 |
| 31025 | 6:37329583 | 31027 | 6:37329948 | 31028 | 6:37330186 | 31029 | 6:37330198 | 31031 | 6:37330365 |
| 31034 | 6:37330605 | 31035 | 6:37330677 | 31036 | 6:37331712 | 31037 | 6:37331772 | 31038 | 6:37331921 |
| 31039 | 6:37332914 | 31040 | 6:37333089 | 31041 | 6:37333257 | 31042 | 6:37333646 | 31043 | 6:37335096 |
| 31044 | 6:37335574 | 31045 | 6:37335884 | 31047 | 6:37336176 | 31048 | 6:37336277 | 31049 | 6:37336431 |
| 31050 | 6:37337044 | 31051 | 6:37337343 | 31053 | 6:37337910 | 31054 | 6:37340194 | 31055 | 6:37340475 |
| 31056 | 6:37340937 | 31057 | 6:37341535 | 31058 | 6:37342568 | 31059 | 6:37342705 | 31060 | 6:37343364 |
| 31061 | 6:37343379 | 31062 | 6:37344246 | 31063 | 6:37344303 | 31064 | 6:37344897 | 31065 | 6:37345259 |
| 31066 | 6:37345508 | 31067 | 6:37345931 | 31068 | 6:37346124 | 31069 | 6:37346442 | 31070 | 6:37347130 |
| 31071 | 6:37349144 | 31072 | 6:37350316 | 31073 | 6:37350368 | 31074 | 6:37351499 | 31075 | 6:37351767 |
| 31076 | 6:37352145 | 31077 | 6:37352282 | 31078 | 6:37352820 | 31079 | 6:37353288 | 31080 | 6:37354945 |
| 31081 | 6:37355132 | 31082 | 6:37355369 | 31083 | 6:37356067 | 31084 | 6:37357055 | 31085 | 6:37358828 |
| 31086 | 6:37359765 | 31087 | 6:37360499 | 31088 | 6:37360685 | 31089 | 6:37361449 | 31090 | 6:37361578 |
| 31092 | 6:37363842 | 31093 | 6:37365265 | 31094 | 6:37365744 | 31095 | 6:37366868 | 31096 | 6:37368371 |
| 31097 | 6:37368652 | 31098 | 6:37368670 | 31100 | 6:37369380 | 31101 | 6:37370421 | 31103 | 6:37373261 |
| 31104 | 6:37373314 | 31105 | 6:37374314 | 31106 | 6:37374718 | 31107 | 6:37376009 | 31109 | 6:37377606 |
| 31110 | 6:37377857 | 31112 | 6:37378824 | 31114 | 6:37379507 | 31115 | 6:37379693 | 31116 | 6:37379729 |
| 31117 | 6:37379796 | 31118 | 6:37380161 | 31119 | 6:37380671 | 31120 | 6:37381106 | 31121 | 6:37381616 |
| 31125 | 6:37382177 | 31126 | 6:37383284 | 31128 | 6:37384356 | 31130 | 6:37390341 | 31131 | 6:37390788 |
| 31132 | 6:37391198 | 31133 | 6:37391915 | 31134 | 6:37392465 | 31135 | 6:37392540 | 31136 | 6:37392957 |
| 31137 | 6:37393564 | 31138 | 6:37393949 | 31140 | 6:37395313 | 31141 | 6:37395750 | 31142 | 6:37395838 |
| 31143 | 6:37396789 | 31144 | 6:37398199 | 31146 | 6:37399852 | 31147 | 6:37400741 | 31148 | 6:37400800 |
| 31149 | 6:37402511 | 31150 | 6:37402964 | 31152 | 6:37403224 | 31153 | 6:37406239 | 31154 | 6:37406485 |
| 31155 | 6:37406729 | 31156 | 6:37407653 | 31157 | 6:37412062 | 31158 | 6:37412935 | 31159 | 6:37414352 |
| 31160 | 6:37414365 | 31161 | 6:37414416 | 31162 | 6:37415367 | 31164 | 6:37416649 | 31165 | 6:37417046 |
| 31167 | 6:37418164 | 31168 | 6:37418421 | 31170 | 6:37418449 | 31171 | 6:37420252 | 31173 | 6:37423505 |
| 31174 | 6:37424248 | 31175 | 6:37425030 | 31176 | 6:37429071 | 31177 | 6:37429595 | 31180 | 6:37431101 |
| 31181 | 6:37432458 | 31182 | 6:37432854 | 31183 | 6:37433502 | 31184 | 6:37433926 | 31185 | 6:37434244 |
| 31186 | 6:37435861 | 31187 | 6:37436146 | 31188 | 6:37436347 | 31189 | 6:37436731 | 31190 | 6:37437130 |
| 31191 | 6:37437538 | 31193 | 6:37438248 | 31194 | 6:37438530 | 31195 | 6:37438575 | 31196 | 6:37438599 |
| 31197 | 6:37438993 | 31198 | 6:37439190 | 31200 | 6:37440955 | 31201 | 6:37441274 | 31202 | 6:37441899 |
| 31203 | 6:37442320 | 31204 | 6:37444900 | 31205 | 6:37446552 | 31206 | 6:37448497 | 31207 | 6:37449358 |
| 31208 | 6:37449890 | 31209 | 6:37450921 | 31210 | 6:37453215 | 31211 | 6:37456193 | 31213 | 6:37461999 |
| 31214 | 6:37463282 | 31215 | 6:37463709 | 31217 | 6:37466194 | 31218 | 6:37473453 | 31219 | 6:37477629 |
| 31220 | 6:37479269 | 31221 | 6:37480277 | 31223 | 6:37483537 | 31224 | 6:37489614 | 31226 | 6:37493760 |
| 31227 | 6:37495347 | 31228 | 6:37500943 | 31230 | 6:37505093 | 31231 | 6:37508184 | 31232 | 6:37511610 |
| 31233 | 6:37512672 | 31234 | 6:37512811 | 31235 | 6:37513645 | 31236 | 6:37520035 | 31237 | 6:37521235 |
| 31238 | 6:37522190 | 31239 | 6:37522580 | 31240 | 6:37526812 | 31241 | 6:37527163 | 31242 | 6:37529800 |
| 31244 | 6:37543134 | 31245 | 6:37547826 | 31247 | 6:37559117 | 31250 | 6:37564896 | 31251 | 6:37566268 |
| 31253 | 6:37566504 | 31255 | 6:37573615 | 31257 | 6:37578899 | 31258 | 6:37579956 | 31259 | 6:37580857 |
| 31260 | 6:37585527 | 31261 | 6:37587677 | 31264 | 6:37591326 | 31266 | 6:37592943 | 31267 | 6:37595517 |
| 31269 | 6:37599145 | 31270 | 6:37599699 | 31271 | 6:37602013 | 31272 | 6:37604468 | 31273 | 6:37606625 |
| 31274 | 6:37607139 | 31275 | 6:37608496 | 31276 | 6:37608504 | 31277 | 6:37614039 | 31278 | 6:37614179 |
| 31279 | 6:37617530 | 31280 | 6:37624003 | 31281 | 6:37625923 | 31282 | 6:37627776 | 31283 | 6:37633434 |
| 31284 | 6:37636883 | 31285 | 6:37636900 | 31286 | 6:37637484 | 31290 | 6:37641448 | 31293 | 6:37644691 |
| 31294 | 6:37644915 | 31295 | 6:37645671 | 31296 | 6:37646000 | 31297 | 6:37646499 | 31298 | 6:37646597 |
| 31299 | 6:37652729 | 31300 | 6:37658289 | 31302 | 6:37664673 | 31303 | 6:37666279 | 31304 | 6:37669453 |
| 31305 | 6:37674317 | 31306 | 6:37675400 | 31307 | 6:37679238 | 31308 | 6:37681968 | 31309 | 6:37683699 |
| 31310 | 6:37687844 | 31312 | 6:37690283 | 31313 | 6:37690852 | 31314 | 6:37696619 | 31315 | 6:37697757 |
| 31316 | 6:37698657 | 31317 | 6:37699059 | 31318 | 6:37703461 | 31319 | 6:37704637 | 31320 | 6:37706431 |
| 31321 | 6:37706528 | 31322 | 6:37708048 | 31323 | 6:37708742 | 31324 | 6:37708953 | 31325 | 6:37709524 |
| 31327 | 6:37712519 | 31328 | 6:37712749 | 31329 | 6:37713097 | 31330 | 6:37713686 | 31331 | 6:37713889 |
| 31332 | 6:37719635 | 31333 | 6:37719804 | 31334 | 6:37720524 | 31335 | 6:37727280 | 31336 | 6:37728142 |
| 31337 | 6:37731195 | 31338 | 6:37736960 | 31339 | 6:37738532 | 31340 | 6:37740667 | 31341 | 6:37741893 |
| 31342 | 6:37742839 | 31343 | 6:37743712 | 31344 | 6:37749840 | 31345 | 6:37749955 | 31346 | 6:37751452 |
| 31347 | 6:37752740 | 31348 | 6:37760883 | 31349 | 6:37772675 | 31350 | 6:37773321 | 31351 | 6:37773355 |
| 31352 | 6:37774136 | 31354 | 6:37775491 | 31355 | 6:37775624 | 31356 | 6:37775822 | 31357 | 6:37776401 |
| 31358 | 6:37776428 | 31359 | 6:37776454 | 31360 | 6:37776464 | 31362 | 6:37777056 | 31363 | 6:37777185 |
| 31364 | 6:37778681 | 31365 | 6:37778924 | 31367 | 6:37797233 | 31368 | 6:37802327 | 31369 | 6:37803937 |
| 31370 | 6:37803941 | 31372 | 6:37804729 | 31373 | 6:37805097 | 31374 | 6:37806070 | 31376 | 6:37809966 |
| 31377 | 6:37810505 | 31378 | 6:37810779 | 31379 | 6:37811541 | 31380 | 6:37811600 | 31381 | 6:37811808 |
| 31382 | 6:37811947 | 31383 | 6:37812109 | 31384 | 6:37812521 | 31385 | 6:37813026 | 31386 | 6:37813294 |
| 31387 | 6:37813424 | 31388 | 6:37813572 | 31389 | 6:37813638 | 31390 | 6:37813728 | 31391 | 6:37813964 |
| 31392 | 6:37814265 | 31393 | 6:37814928 | 31394 | 6:37815457 | 31395 | 6:37815489 | 31396 | 6:37815615 |
| 31397 | 6:37815806 | 31398 | 6:37815918 | 31399 | 6:37816011 | 31400 | 6:37816192 | 31401 | 6:37816231 |
| 31402 | 6:37816300 | 31403 | 6:37817310 | 31405 | 6:37817589 | 31406 | 6:37817855 | 31407 | 6:37817875 |
| 31408 | 6:37817910 | 31409 | 6:37818107 | 31410 | 6:37818111 | 31411 | 6:37818195 | 31412 | 6:37818372 |
| 31413 | 6:37819224 | 31414 | 6:37821523 | 31415 | 6:37821595 | 31416 | 6:37821964 | 31417 | 6:37830099 |
| 31418 | 6:37831626 | 31419 | 6:37833439 | 31420 | 6:37838622 | 31421 | 6:37842392 | 31422 | 6:37895331 |
| 31423 | 6:37902755 | 31424 | 6:37905296 | 31425 | 6:37905467 | 31426 | 6:37907357 | 31427 | 6:37908478 |
| 31428 | 6:37908507 | 31429 | 6:37908679 | 31430 | 6:37908861 | 31431 | 6:37908962 | 31432 | 6:37909143 |
| 31433 | 6:37910276 | 31434 | 6:37910464 | 31435 | 6:37910478 | 31436 | 6:37910866 | 31437 | 6:37911814 |
| 31438 | 6:37912116 | 31439 | 6:37913125 | 31440 | 6:37913652 | 31441 | 6:37913727 | 31442 | 6:37913922 |
| 31443 | 6:37913940 | 31444 | 6:37914439 | 31446 | 6:37914513 | 31447 | 6:37914730 | 31448 | 6:37914816 |
| 31449 | 6:37914822 | 31450 | 6:37914832 | 31451 | 6:37915050 | 31452 | 6:37915308 | 31453 | 6:37916419 |
| 31454 | 6:37917366 | 31455 | 6:37917421 | 31456 | 6:37917796 | 31457 | 6:37918546 | 31458 | 6:37918824 |
| 31459 | 6:37919045 | 31460 | 6:37919095 | 31461 | 6:37919120 | 31462 | 6:37919520 | 31463 | 6:37919631 |
| 31464 | 6:37919661 | 31465 | 6:37919853 | 31466 | 6:37919862 | 31467 | 6:37920305 | 31468 | 6:37920538 |
| 31469 | 6:37920551 | 31470 | 6:37920666 | 31471 | 6:37921510 | 31472 | 6:37921603 | 31474 | 6:37921724 |
| 31476 | 6:37926600 | 31477 | 6:37926960 | 31478 | 6:37928156 | 31479 | 6:37928479 | 31480 | 6:37929065 |
| 31481 | 6:37929104 | 31482 | 6:37929184 | 31483 | 6:37929689 | 31484 | 6:37929786 | 31485 | 6:37930046 |
| 31487 | 6:37930293 | 31488 | 6:37930672 | 31489 | 6:37930781 | 31490 | 6:37930868 | 31491 | 6:37930889 |
| 31492 | 6:37931089 | 31493 | 6:37931216 | 31494 | 6:37931298 | 31495 | 6:37931621 | 31496 | 6:37931688 |
| 31497 | 6:37931726 | 31498 | 6:37931903 | 31499 | 6:37932170 | 31500 | 6:37932197 | 31501 | 6:37932241 |
| 31502 | 6:37932371 | 31503 | 6:37932377 | 31504 | 6:37932412 | 31505 | 6:37932434 | 31506 | 6:37932563 |
| 31508 | 6:37932772 | 31509 | 6:37932778 | 31510 | 6:37932960 | 31511 | 6:37933250 | 31512 | 6:37933352 |
| 31513 | 6:37933965 | 31514 | 6:37934068 | 31515 | 6:37934272 | 31516 | 6:37934993 | 31517 | 6:37935069 |
| 31518 | 6:37935108 | 31519 | 6:37935168 | 31520 | 6:37935527 | 31521 | 6:37935577 | 31522 | 6:37935748 |
| 31523 | 6:37936044 | 31524 | 6:37936087 | 31525 | 6:37936692 | 31526 | 6:37938294 | 31529 | 6:37942096 |
| 31530 | 6:37942170 | 31531 | 6:37942290 | 31532 | 6:37942944 | 31533 | 6:37943635 | 31534 | 6:37943860 |
| 31536 | 6:37944478 | 31537 | 6:37944482 | 31538 | 6:37944496 | 31539 | 6:37944573 | 31540 | 6:37944631 |
| 31541 | 6:37944681 | 31542 | 6:37944704 | 31543 | 6:37945333 | 31544 | 6:37945482 | 31545 | 6:37945820 |
| 31546 | 6:37945849 | 31547 | 6:37946012 | 31548 | 6:37946384 | 31549 | 6:37953254 | 31550 | 6:37953301 |
| 31551 | 6:37953591 | 31552 | 6:37954699 | 31553 | 6:37954789 | 31554 | 6:37954873 | 31555 | 6:37955372 |
| 31556 | 6:37956368 | 31557 | 6:37956390 | 31558 | 6:37959360 | 31559 | 6:37960313 | 31560 | 6:37961491 |
| 31561 | 6:37961553 | 31562 | 6:37962093 | 31563 | 6:37962887 | 31564 | 6:37963346 | 31565 | 6:37963525 |
| 31566 | 6:37965259 | 31567 | 6:37965489 | 31568 | 6:37966382 | 31569 | 6:37968595 | 31570 | 6:37970172 |
| 31571 | 6:37970860 | 31572 | 6:37971218 | 31573 | 6:37971544 | 31574 | 6:37971810 | 31575 | 6:37971997 |
| 31576 | 6:37972173 | 31577 | 6:37972335 | 31578 | 6:37972414 | 31579 | 6:37972642 | 31580 | 6:37972906 |
| 31581 | 6:37973011 | 31582 | 6:37973508 | 31583 | 6:37973804 | 31584 | 6:37974302 | 31586 | 6:37974407 |
| 31587 | 6:37974974 | 31588 | 6:37976818 | 31589 | 6:37978037 | 31590 | 6:37978995 | 31591 | 6:37979083 |
| 31592 | 6:37981093 | 31593 | 6:37981125 | 31594 | 6:37983153 | 31595 | 6:37984808 | 31596 | 6:37984948 |
| 31597 | 6:37985243 | 31598 | 6:37987337 | 31599 | 6:37987503 | 31600 | 6:37989831 | 31601 | 6:37990674 |
| 31602 | 6:37990808 | 31603 | 6:37991751 | 31604 | 6:37992856 | 31605 | 6:37993125 | 31606 | 6:37993517 |
| 31607 | 6:37993754 | 31608 | 6:37994046 | 31609 | 6:37996786 | 31610 | 6:37996859 | 31611 | 6:37997160 |
| 31612 | 6:37997516 | 31613 | 6:37998187 | 31614 | 6:37998452 | 31615 | 6:37998592 | 31616 | 6:37998656 |
| 31617 | 6:37998696 | 31618 | 6:37999740 | 31619 | 6:37999825 | 31620 | 6:37999842 | 31621 | 6:38000343 |
| 31622 | 6:38000353 | 31623 | 6:38001392 | 31624 | 6:38001622 | 31625 | 6:38002117 | 31626 | 6:38002596 |
| 31627 | 6:38004124 | 31628 | 6:38004325 | 31629 | 6:38005805 | 31630 | 6:38007975 | 31631 | 6:38014109 |
| 31632 | 6:38016521 | 31633 | 6:38017827 | 31634 | 6:38017866 | 31635 | 6:38021694 | 31636 | 6:38022226 |
| 31637 | 6:38022305 | 31638 | 6:38023218 | 31639 | 6:38023327 | 31640 | 6:38023545 | 31641 | 6:38023613 |
| 31642 | 6:38023673 | 31643 | 6:38023878 | 31644 | 6:38024011 | 31645 | 6:38024702 | 31646 | 6:38024768 |
| 31647 | 6:38025411 | 31648 | 6:38025684 | 31649 | 6:38025734 | 31650 | 6:38026545 | 31651 | 6:38027078 |
| 31652 | 6:38027106 | 31653 | 6:38027129 | 31654 | 6:38031018 | 31655 | 6:38032275 | 31656 | 6:38033896 |
| 31658 | 6:38035001 | 31659 | 6:38035414 | 31660 | 6:38035460 | 31661 | 6:38035561 | 31662 | 6:38035568 |
| 31663 | 6:38035625 | 31664 | 6:38035705 | 31665 | 6:38036061 | 31666 | 6:38036351 | 31667 | 6:38036588 |
| 31668 | 6:38036594 | 31669 | 6:38036635 | 31670 | 6:38036660 | 31671 | 6:38036882 | 31672 | 6:38037099 |
| 31673 | 6:38037535 | 31674 | 6:38037724 | 31675 | 6:38037768 | 31676 | 6:38038091 | 31677 | 6:38038189 |
| 31678 | 6:38038404 | 31679 | 6:38038608 | 31680 | 6:38039088 | 31681 | 6:38039492 | 31682 | 6:38040456 |
| 31683 | 6:38040488 | 31684 | 6:38040529 | 31685 | 6:38040769 | 31686 | 6:38040934 | 31687 | 6:38041234 |
| 31688 | 6:38041593 | 31689 | 6:38041633 | 31690 | 6:38041715 | 31691 | 6:38042205 | 31692 | 6:38042337 |
| 31693 | 6:38043977 | 31694 | 6:38044618 | 31695 | 6:38044876 | 31696 | 6:38044898 | 31697 | 6:38045727 |
| 31698 | 6:38046428 | 31699 | 6:38048735 | 31700 | 6:38051406 | 31701 | 6:38051509 | 31702 | 6:38052049 |
| 31703 | 6:38052245 | 31704 | 6:38054733 | 31706 | 6:38063490 | 31707 | 6:38064317 | 31708 | 6:38065703 |
| 31709 | 6:38066309 | 31710 | 6:38067289 | 31711 | 6:38067376 | 31712 | 6:38067855 | 31713 | 6:38068311 |
| 31714 | 6:38068956 | 31715 | 6:38069662 | 31716 | 6:38070361 | 31717 | 6:38070496 | 31718 | 6:38070882 |
| 31719 | 6:38071318 | 31720 | 6:38071414 | 31721 | 6:38071547 | 31722 | 6:38073914 | 31723 | 6:38076161 |
| 31724 | 6:38076905 | 31725 | 6:38077237 | 31726 | 6:38077323 | 31728 | 6:38078774 | 31729 | 6:38079456 |
| 31730 | 6:38079499 | 31731 | 6:38082355 | 31732 | 6:38085803 | 31733 | 6:38087237 | 31734 | 6:38087636 |
| 31735 | 6:38089862 | 31736 | 6:38094533 | 31737 | 6:38097987 | 31739 | 6:38107020 | 31740 | 6:38108493 |
| 31742 | 6:38114043 | 31743 | 6:38114047 | 31744 | 6:38114717 | 31745 | 6:38114803 | 31746 | 6:38114808 |
| 31747 | 6:38115023 | 31748 | 6:38118015 | 31749 | 6:38122903 | 31750 | 6:38123039 | 31751 | 6:38127700 |
| 31752 | 6:38131158 | 31753 | 6:38132133 | 31754 | 6:38132688 | 31755 | 6:38135706 | 31756 | 6:38137640 |
| 31757 | 6:38137799 | 31758 | 6:38138017 | 31759 | 6:38138089 | 31760 | 6:38139495 | 31761 | 6:38139563 |
| 31762 | 6:38139762 | 31763 | 6:38140108 | 31764 | 6:38140123 | 31765 | 6:38140536 | 31766 | 6:38140681 |
| 31767 | 6:38141284 | 31768 | 6:38141304 | 31769 | 6:38141629 | 31770 | 6:38141696 | 31771 | 6:38141833 |
| 31772 | 6:38142148 | 31773 | 6:38142807 | 31774 | 6:38142835 | 31775 | 6:38142840 | 31776 | 6:38144120 |
| 31777 | 6:38144195 | 31778 | 6:38144291 | 31779 | 6:38144317 | 31780 | 6:38144587 | 31781 | 6:38156378 |
| 31782 | 6:38156469 | 31783 | 6:38156591 | 31784 | 6:38157637 | 31785 | 6:38157684 | 31786 | 6:38158107 |
| 31787 | 6:38158113 | 31791 | 6:38199691 | 31792 | 6:38202499 | 31793 | 6:38211198 | 31795 | 6:38227779 |
| 31797 | 6:38237566 | 31798 | 6:38237695 | 31799 | 6:38247802 | 31800 | 6:38252776 | 31805 | 6:38278657 |
| 31806 | 6:38278666 | 31807 | 6:38278782 | 31808 | 6:38279030 | 31809 | 6:38281274 | 31814 | 6:38318628 |
| 31817 | 6:38375856 | 31820 | 6:38396789 | 31823 | 6:38409609 | 31825 | 6:38433243 | 31827 | 6:38499580 |
| 31828 | 6:38500871 | 31829 | 6:38506361 | 31830 | 6:38508464 | 31832 | 6:38519250 | 31833 | 6:38525790 |
| 31834 | 6:38535911 | 31835 | 6:38540318 | 31838 | 6:38541639 | 31840 | 6:38542150 | 31844 | 6:38543990 |
| 31848 | 6:38546646 | 31849 | 6:38548060 | 31850 | 6:38548198 | 31854 | 6:38549044 | 31856 | 6:38549206 |
| 31858 | 6:38549703 | 31859 | 6:38550162 | 31869 | 6:38553181 | 31881 | 6:38556220 | 31895 | 6:38562229 |
| 31896 | 6:38563063 | 31898 | 6:38565084 | 31899 | 6:38566242 | 31900 | 6:38570744 | 31901 | 6:38575762 |
| 31903 | 6:38584420 | 31904 | 6:38584686 | 31905 | 6:38588303 | 31906 | 6:38599441 | 31907 | 6:38600028 |
| 31908 | 6:38600235 | 31909 | 6:38602696 | 31910 | 6:38617268 | 31911 | 6:38617890 | 31912 | 6:38618593 |
| 31913 | 6:38619398 | 31914 | 6:38619754 | 31915 | 6:38619795 | 31916 | 6:38620014 | 31917 | 6:38620218 |
| 31918 | 6:38621930 | 31919 | 6:38624000 | 31920 | 6:38624126 | 31921 | 6:38644939 | 31923 | 6:38695951 |
| 31924 | 6:38697400 | 31928 | 6:38789984 | 31929 | 6:38798194 | 31933 | 6:38854750 | 31942 | 6:38907049 |
| 31943 | 6:38918524 | 31945 | 6:38919029 | 31946 | 6:38929469 | 31947 | 6:38933534 | 31948 | 6:38934821 |
| 31950 | 6:38963229 | 31951 | 6:38973635 | 31952 | 6:38973995 | 31953 | 6:38980873 | 31954 | 6:39030259 |
| 31956 | 6:39082331 | 31957 | 6:39109137 | 31962 | 6:39181262 | 31966 | 6:39254458 | 31968 | 6:39280700 |
| 31974 | 6:39437165 | 31977 | 6:39518939 | 31978 | 6:39532147 | 31979 | 6:39559751 | 31982 | 6:39574982 |
| 31984 | 6:39595691 | 31985 | 6:39595731 | 31988 | 6:39652421 | 31992 | 6:39721697 | 31994 | 6:39730068 |
| 31995 | 6:39733610 | 31996 | 6:39737879 | 31998 | 6:39755798 | 31999 | 6:39756006 | 32002 | 6:39760650 |
| 32003 | 6:39772834 | 32004 | 6:39772963 | 32005 | 6:39773133 | 32007 | 6:39797151 | 32009 | 6:39825526 |
| 32011 | 6:39858450 | 32012 | 6:39874821 | 32013 | 6:39885647 | 32026 | 6:40072649 | 32030 | 6:40130949 |
| 32032 | 6:40203593 | 32035 | 6:40239109 | 32036 | 6:40248052 | 32042 | 6:40338033 | 32047 | 6:40400197 |
| 32048 | 6:40432849 | 32049 | 6:40456680 | 32052 | 6:40565574 | 32055 | 6:40599155 | 32058 | 6:40636686 |
| 32059 | 6:40662051 | 32060 | 6:40672232 | 32061 | 6:40689118 | 32062 | 6:40696320 | 32085 | 6:40731922 |
| 32089 | 6:40758035 | 32093 | 6:40780850 | 32094 | 6:40808612 | 32098 | 6:40836283 | 32102 | 6:40911873 |
| 32103 | 6:40920367 | 32104 | 6:40921708 | 32105 | 6:40933517 | 32106 | 6:40935693 | 32107 | 6:40935713 |
| 32108 | 6:40935857 | 32109 | 6:40935890 | 32110 | 6:40936031 | 32111 | 6:40936207 | 32112 | 6:40936326 |
| 32113 | 6:40936831 | 32114 | 6:40936837 | 32115 | 6:40937031 | 32116 | 6:40937441 | 32117 | 6:40942588 |
| 32120 | 6:40984868 | 32121 | 6:40988645 | 32122 | 6:40988913 | 32123 | 6:40999090 | 32124 | 6:41003445 |
| 32125 | 6:41004894 | 32126 | 6:41029896 | 32127 | 6:41033792 | 32128 | 6:41049370 | 32129 | 6:41049496 |
| 32130 | 6:41049859 | 32131 | 6:41049914 | 32132 | 6:41050127 | 32133 | 6:41050140 | 32135 | 6:41050862 |
| 32136 | 6:41051120 | 32137 | 6:41053625 | 32138 | 6:41061635 | 32139 | 6:41073550 | 32140 | 6:41079462 |
| 32142 | 6:41088062 | 32143 | 6:41090326 | 32144 | 6:41096500 | 32145 | 6:41107449 | 32146 | 6:41107836 |
| 32147 | 6:41108192 | 32148 | 6:41108818 | 32149 | 6:41108912 | 32150 | 6:41108926 | 32151 | 6:41109137 |
| 32152 | 6:41109217 | 32153 | 6:41109350 | 32155 | 6:41150218 | 32156 | 6:41169318 | 32157 | 6:41170432 |
| 32158 | 6:41173967 | 32161 | 6:41185741 | 32162 | 6:41204988 | 32169 | 6:41306166 | 32170 | 6:41306273 |
| 32171 | 6:41306284 | 32172 | 6:41306424 | 32173 | 6:41308067 | 32174 | 6:41331258 | 32179 | 6:41577847 |
| 32180 | 6:41592300 | 32181 | 6:41677493 | 32188 | 6:41868518 | 32195 | 6:42004652 | 32203 | 6:42037316 |
| 32209 | 6:42163671 | 32216 | 6:42441818 | 32220 | 6:42507082 | 32221 | 6:42510330 | 32227 | 6:42559603 |
| 32229 | 6:42665842 | 32230 | 6:42672683 | 32231 | 6:42722868 | 32232 | 6:42724776 | 32233 | 6:42728138 |
| 32234 | 6:42741244 | 32235 | 6:42741447 | 32236 | 6:42758331 | 32241 | 6:42837669 | 32263 | 6:42974326 |
| 32284 | 6:43317249 | 32296 | 6:43494887 | 32298 | 6:43544441 | 32303 | 6:43601154 | 32309 | 6:43679917 |
| 32318 | 6:43822090 | 32319 | 6:43822403 | 32320 | 6:43829217 | 32323 | 6:43886543 | 32326 | 6:43944737 |
| 32330 | 6:43983354 | 32331 | 6:44003666 | 32332 | 6:44026498 | 32336 | 6:44072335 | 32339 | 6:44149286 |
| 32343 | 6:44276184 | 32360 | 6:44628911 | 32362 | 6:44678042 | 32363 | 6:44685469 | 32364 | 6:44686809 |
| 32365 | 6:44689379 | 32366 | 6:44690759 | 32368 | 6:44694506 | 32369 | 6:44694605 | 32370 | 6:44695255 |
| 32371 | 6:44695433 | 32372 | 6:44695563 | 32373 | 6:44695784 | 32374 | 6:44695872 | 32375 | 6:44695978 |
| 32376 | 6:44695987 | 32377 | 6:44696022 | 32378 | 6:44696578 | 32379 | 6:44696796 | 32380 | 6:44696931 |
| 32381 | 6:44696955 | 32382 | 6:44698160 | 32383 | 6:44704061 | 32384 | 6:44704410 | 32385 | 6:44705240 |
| 32386 | 6:44706199 | 32387 | 6:44706279 | 32388 | 6:44709016 | 32389 | 6:44709754 | 32390 | 6:44710003 |
| 32391 | 6:44710237 | 32392 | 6:44710415 | 32393 | 6:44710515 | 32394 | 6:44711099 | 32395 | 6:44712295 |
| 32396 | 6:44735374 | 32397 | 6:44736099 | 32398 | 6:44739055 | 32399 | 6:44742360 | 32400 | 6:44747933 |
| 32401 | 6:44748956 | 32403 | 6:44775326 | 32404 | 6:44777357 | 32405 | 6:44784742 | 32406 | 6:44787261 |
| 32407 | 6:44791513 | 32408 | 6:44791744 | 32409 | 6:44797875 | 32410 | 6:44809081 | 32411 | 6:44810108 |
| 32412 | 6:44818322 | 32413 | 6:44819710 | 32414 | 6:44820115 | 32415 | 6:44823097 | 32416 | 6:44825630 |
| 32418 | 6:44828213 | 32419 | 6:44831889 | 32420 | 6:44857705 | 32421 | 6:44864378 | 32422 | 6:44873514 |
| 32423 | 6:44873843 | 32424 | 6:44874186 | 32425 | 6:44877670 | 32426 | 6:44883344 | 32427 | 6:44893464 |
| 32428 | 6:44904257 | 32429 | 6:44905170 | 32430 | 6:44909660 | 32431 | 6:44914656 | 32432 | 6:44920744 |
| 32433 | 6:44931581 | 32434 | 6:44934298 | 32435 | 6:44934689 | 32437 | 6:44983137 | 32439 | 6:45018531 |
| 32440 | 6:45018882 | 32441 | 6:45029390 | 32442 | 6:45029425 | 32443 | 6:45029626 | 32444 | 6:45030486 |
| 32445 | 6:45030599 | 32446 | 6:45030709 | 32447 | 6:45030774 | 32448 | 6:45030789 | 32449 | 6:45031327 |
| 32450 | 6:45031721 | 32451 | 6:45031956 | 32452 | 6:45032017 | 32453 | 6:45032153 | 32454 | 6:45033264 |
| 32455 | 6:45037315 | 32456 | 6:45037561 | 32457 | 6:45037753 | 32458 | 6:45037798 | 32459 | 6:45038091 |
| 32460 | 6:45038647 | 32461 | 6:45038676 | 32462 | 6:45038869 | 32463 | 6:45040756 | 32464 | 6:45041754 |
| 32465 | 6:45043489 | 32466 | 6:45043654 | 32467 | 6:45043859 | 32468 | 6:45043984 | 32469 | 6:45044149 |
| 32470 | 6:45045596 | 32471 | 6:45048213 | 32472 | 6:45048323 | 32473 | 6:45049586 | 32474 | 6:45053716 |
| 32475 | 6:45054332 | 32476 | 6:45055326 | 32477 | 6:45057776 | 32478 | 6:45057805 | 32479 | 6:45058034 |
| 32480 | 6:45058273 | 32481 | 6:45077729 | 32485 | 6:45135036 | 32487 | 6:45183436 | 32489 | 6:45185710 |
| 32490 | 6:45190122 | 32491 | 6:45205382 | 32492 | 6:45213648 | 32497 | 6:45274484 | 32500 | 6:45322366 |
| 32502 | 6:45325845 | 32505 | 6:45364629 | 32518 | 6:45583636 | 32519 | 6:45595528 | 32526 | 6:45885499 |
| 32569 | 6:46705772 | 32576 | 6:46946921 | 32580 | 6:47016991 | 32586 | 6:47147481 | 32587 | 6:47164702 |
| 32591 | 6:47219584 | 32592 | 6:47256173 | 32594 | 6:47317499 | 32596 | 6:47365064 | 32599 | 6:47424087 |
| 32602 | 6:47570425 | 32614 | 6:47825353 | 32615 | 6:47843766 | 32616 | 6:47866852 | 32621 | 6:47989356 |
| 32623 | 6:48030364 | 32626 | 6:48149995 | 32648 | 6:48893818 | 32652 | 6:49019297 | 32655 | 6:49094600 |
| 32662 | 6:49295440 | 32679 | 6:49701147 | 32694 | 6:49848545 | 32696 | 6:49878456 | 32701 | 6:50009636 |
| 32703 | 6:50090470 | 32704 | 6:50126302 | 32709 | 6:50252554 | 32716 | 6:50385741 | 32717 | 6:50412585 |
| 32719 | 6:50455132 | 32724 | 6:50672369 | 32729 | 6:50779278 | 32731 | 6:50856440 | 32748 | 6:51325072 |
| 32750 | 6:51362285 | 32759 | 6:51740645 | 32778 | 6:52250315 | 32780 | 6:52275948 | 32781 | 6:52320441 |
| 32782 | 6:52339267 | 32783 | 6:52339325 | 32784 | 6:52347811 | 32785 | 6:52347899 | 32786 | 6:52347995 |
| 32787 | 6:52348073 | 32788 | 6:52348287 | 32789 | 6:52348292 | 32790 | 6:52348643 | 32791 | 6:52348674 |
| 32792 | 6:52349023 | 32793 | 6:52349157 | 32794 | 6:52349544 | 32795 | 6:52349562 | 32796 | 6:52349794 |
| 32797 | 6:52350003 | 32798 | 6:52350227 | 32799 | 6:52350240 | 32800 | 6:52351181 | 32801 | 6:52351388 |
| 32802 | 6:52352446 | 32817 | 6:52645662 | 32818 | 6:52671724 | 32825 | 6:52864424 | 32829 | 6:52954024 |
| 32831 | 6:53024244 | 32842 | 6:53220820 | 32846 | 6:53285747 | 32847 | 6:53314886 | 32850 | 6:53397301 |
| 32853 | 6:53471919 | 32862 | 6:53752557 | 32873 | 6:54004873 | 32874 | 6:54031927 | 32877 | 6:54086328 |
| 32879 | 6:54185052 | 32897 | 6:54783996 | 32913 | 6:55303674 | 32920 | 6:55450495 | 32941 | 6:55514144 |
| 32976 | 6:56317455 | 32994 | 6:56900473 | 33006 | 6:57179884 | 33012 | 6:57260380 | 33031 | 6:57842883 |
| 33035 | 6:57984344 | 33039 | 6:58077967 | 33066 | 6:58746181 | 33077 | 6:59130010 | 33095 | 6:59579872 |
| 33097 | 6:59716766 | 33111 | 6:59875075 | 33114 | 6:59907442 | 33122 | 6:60085027 | 33129 | 6:60257426 |
| 33137 | 6:60400323 | 33155 | 6:60802613 | 33168 | 6:61185257 | 33176 | 6:61390096 | 33183 | 6:61487026 |
| 33204 | 6:62234468 | 33223 | 6:62814916 | 33224 | 6:62862206 | 33225 | 6:62889960 | 33227 | 6:62925583 |
| 33238 | 6:63122811 | 33240 | 6:63170295 | 33242 | 6:63209301 | 33244 | 6:63244734 | 33260 | 6:63836353 |
| 33307 | 6:65456807 | 33308 | 6:65520848 | 33338 | 6:66507621 | 33350 | 6:66896326 | 33361 | 6:67256076 |
| 33376 | 6:67767947 | 33381 | 6:67991798 | 33394 | 6:68331252 | 33397 | 6:68416278 | 33418 | 6:68896760 |
| 33422 | 6:69030825 | 33433 | 6:69252356 | 33435 | 6:69346909 | 33444 | 6:69724839 | 33474 | 6:70673035 |
| 33478 | 6:70789469 | 33484 | 6:70978405 | 33516 | 6:72022899 | 33530 | 6:72349944 | 33534 | 6:72385883 |
| 33535 | 6:72419275 | 33544 | 6:72713559 | 33582 | 6:74091185 | 33584 | 6:74174913 | 33607 | 6:75066888 |
| 33608 | 6:75102649 | 33667 | 6:76880221 | 33698 | 6:77967685 | 33734 | 6:79446058 | 33751 | 6:80043649 |
| 33764 | 6:80444445 | 33798 | 6:81507132 | 33851 | 6:83098669 | 33875 | 6:83547735 | 33897 | 6:83979787 |
| 33921 | 6:84628765 | 33933 | 6:85067620 | 33936 | 6:85092523 | 33946 | 6:85177598 | 33953 | 6:85422786 |
| 33964 | 6:85548797 | 34000 | 6:86388176 | 34002 | 6:86478657 | 34055 | 6:87399244 | 34089 | 6:88024804 |
| 34126 | 6:89007570 | 34128 | 6:89048060 | 34273 | 6:91629835 | 34306 | 6:92470502 | 34307 | 6:92504637 |
| 34410 | 6:94755352 | 34412 | 6:94776615 | 34452 | 6:96090013 | 34453 | 6:96097237 | 34478 | 6:96544331 |
| 34596 | 6:98671599 | 34598 | 6:98696365 | 34635 | 6:99753620 | 34640 | 6:99898377 | 34644 | 6:100026934 |
| 34702 | 6:101156203 | 34718 | 6:101492004 | 34738 | 6:101904289 | 34742 | 6:101951985 | 34771 | 6:102466503 |
| 34774 | 6:102525990 | 34781 | 6:102622674 | 34936 | 6:105596054 | 35002 | 6:107330690 | 35294 | 6:113769965 |
| 35299 | 6:113826996 | 35354 | 6:114785574 | 35370 | 6:115107065 | 35390 | 6:115648265 | 35391 | 6:115670310 |
| 35405 | 6:115976356 | 35426 | 6:116325904 | 35456 | 6:117124190 | 35476 | 7:210754 | 35491 | 7:521674 |
| 35511 | 7:987326 | 35515 | 7:1068152 | 35516 | 7:1068170 | 35638 | 7:3108200 | 35680 | 7:3821837 |
| 35729 | 7:5027580 | 35741 | 7:5125016 | 35859 | 7:6346703 | 35871 | 7:6483779 | 36139 | 7:13283360 |
| 36142 | 7:13315974 | 36174 | 7:13946048 | 36270 | 7:15409865 | 36316 | 7:16530067 | 36366 | 7:17643702 |
| 36378 | 7:17996900 | 36554 | 7:21064478 | 36624 | 7:22566377 | 36628 | 7:22594727 | 36656 | 7:23523707 |
| 36685 | 7:24072015 | 36686 | 7:24142269 | 36697 | 7:24434170 | 36698 | 7:24436989 | 36816 | 7:26920631 |
| 36863 | 7:27929188 | 36943 | 7:29802791 | 36954 | 7:30036916 | 36957 | 7:30105269 | 37015 | 7:31343335 |
| 37063 | 7:32305922 | 37066 | 7:32334690 | 37099 | 7:33229945 | 37111 | 7:33525039 | 37122 | 7:33761341 |
| 37149 | 7:34411840 | 37156 | 7:34553590 | 37176 | 7:35028517 | 37430 | 7:40687768 | 37528 | 7:43355224 |
| 37570 | 7:44672168 | 37748 | 7:48982387 | 37783 | 7:50794543 | 37816 | 7:52176483 | 37833 | 7:52224828 |
| 37838 | 7:52249785 | 38015 | 7:56078682 | 38041 | 7:56774182 | 38048 | 7:56997351 | 38071 | 7:57843022 |
| 38075 | 7:57875535 | 38208 | 7:60668104 | 38263 | 7:61497597 | 38359 | 7:63355823 | 38427 | 7:65211026 |
| 38428 | 7:65229182 | 38429 | 7:65236569 | 38430 | 7:65246212 | 38431 | 7:65252605 | 38470 | 7:66072980 |
| 38560 | 7:67858059 | 38734 | 7:72087070 | 38741 | 7:72254021 | 38780 | 7:73605674 | 38781 | 7:73607632 |
| 38782 | 7:73637074 | 38785 | 7:73667360 | 38867 | 7:75759697 | 38875 | 7:75832195 | 38885 | 7:76174417 |
| 38922 | 7:77054436 | 38942 | 7:77465055 | 38993 | 7:78445222 | 38999 | 7:78709632 | 39099 | 7:80473773 |
| 39124 | 7:80863489 | 39132 | 7:81093522 | 39153 | 7:81316371 | 39179 | 7:81968933 | 39185 | 7:82214691 |
| 39191 | 7:82275842 | 39194 | 7:82369878 | 39298 | 7:85032366 | 39351 | 7:86627973 | 39378 | 7:87376691 |
| 39402 | 7:88102064 | 39416 | 7:88541691 | 39453 | 7:89335556 | 39457 | 7:89404622 | 39489 | 7:90218611 |
| 39512 | 7:90622467 | 39514 | 7:90688919 | 39518 | 7:90701213 | 39520 | 7:90747139 | 39521 | 7:90755504 |
| 39522 | 7:90756120 | 39526 | 7:90805595 | 39527 | 7:90807032 | 39528 | 7:90807192 | 39529 | 7:90807464 |
| 39530 | 7:90807690 | 39531 | 7:90807800 | 39532 | 7:90807968 | 39533 | 7:90808002 | 39534 | 7:90808188 |
| 39535 | 7:90808858 | 39536 | 7:90813061 | 39537 | 7:90813398 | 39539 | 7:90818194 | 39540 | 7:90818916 |
| 39541 | 7:90819613 | 39542 | 7:90820830 | 39543 | 7:90821093 | 39544 | 7:90821542 | 39545 | 7:90821986 |
| 39546 | 7:90822179 | 39547 | 7:90822452 | 39548 | 7:90822601 | 39549 | 7:90823320 | 39550 | 7:90824804 |
| 39551 | 7:90825013 | 39552 | 7:90825646 | 39553 | 7:90825779 | 39554 | 7:90825887 | 39555 | 7:90827178 |
| 39557 | 7:90831014 | 39562 | 7:90842614 | 39567 | 7:90851664 | 39571 | 7:90889577 | 39597 | 7:91489258 |
| 39602 | 7:91611232 | 39603 | 7:91625156 | 39604 | 7:91625242 | 39605 | 7:91625323 | 39606 | 7:91625348 |
| 39609 | 7:91710576 | 39613 | 7:91786432 | 39615 | 7:91796723 | 39616 | 7:91818525 | 39624 | 7:91975067 |
| 39702 | 7:94438004 | 39707 | 7:94529183 | 39744 | 7:95454269 | 39752 | 7:95607084 | 39792 | 7:96165472 |
| 39942 | 7:96409276 | 39956 | 7:96720179 | 39962 | 7:96807889 | 39981 | 7:97434118 | 39990 | 7:97603739 |
| 40255 | 7:104401635 | 40270 | 7:104812484 | 40304 | 7:105679720 | 40323 | 7:106127282 | 40332 | 7:106311771 |
| 40335 | 7:106392644 | 40416 | 7:108497744 | 40421 | 7:108592553 | 40429 | 7:108781177 | 40437 | 7:108939690 |
| 40523 | 8:905854 | 40531 | 8:972589 | 40654 | 8:3953653 | 40665 | 8:4218936 | 40702 | 8:5114851 |
| 40704 | 8:5164202 | 40769 | 8:6831018 | 41003 | 8:12159615 | 41129 | 8:15805194 | 41141 | 8:16218371 |
| 41181 | 8:17034771 | 41209 | 8:17591195 | 41218 | 8:17834202 | 41244 | 8:18677218 | 41417 | 8:23907022 |
| 41564 | 8:25653708 | 41594 | 8:26211730 | 41629 | 8:27285102 | 41727 | 8:29969758 | 41835 | 8:33203967 |
| 41881 | 8:34438846 | 41903 | 8:35034999 | 41929 | 8:35568258 | 41956 | 8:36096463 | 41973 | 8:36469080 |
| 41974 | 8:36489911 | 42016 | 8:37579398 | 42038 | 8:38343034 | 42071 | 8:39659087 | 42104 | 8:40219724 |
| 42149 | 8:41244408 | 42172 | 8:41710832 | 42173 | 8:41735284 | 42187 | 8:42123936 | 42190 | 8:42204721 |
| 42208 | 8:42686594 | 42212 | 8:42720374 | 42216 | 8:42880518 | 42241 | 8:43615936 | 42271 | 8:44484817 |
| 42312 | 8:45608581 | 42314 | 8:45672590 | 42329 | 8:45953160 | 42330 | 8:45985343 | 42332 | 8:46013064 |
| 42395 | 8:47420937 | 42418 | 8:47998648 | 42466 | 8:49248066 | 42519 | 8:50846856 | 42524 | 8:50995576 |
| 42565 | 8:52018600 | 42588 | 8:52629220 | 42591 | 8:52674907 | 42603 | 8:52986604 | 42661 | 8:54554681 |
| 42683 | 8:55077468 | 42694 | 8:55493961 | 42765 | 8:57313074 | 42849 | 8:59626198 | 42853 | 8:59747078 |
| 42916 | 8:61134883 | 42918 | 8:61212042 | 42919 | 8:61235958 | 42922 | 8:61366316 | 42925 | 8:61452677 |
| 43033 | 8:64222797 | 43049 | 8:64876145 | 43067 | 8:65260788 | 43080 | 8:65506614 | 43087 | 8:65773835 |
| 43124 | 8:66668352 | 43200 | 8:68399674 | 43235 | 8:69120902 | 43284 | 8:70429214 | 43306 | 8:70893052 |
| 43311 | 8:70996588 | 43380 | 8:72930313 | 43454 | 8:74772105 | 43457 | 8:74801687 | 43527 | 8:76729847 |
| 43563 | 8:77557071 | 43702 | 8:81553047 | 43722 | 8:82063613 | 43764 | 8:82948559 | 43831 | 8:84602590 |
| 43865 | 8:85540348 | 43883 | 8:85967740 | 43942 | 8:87290730 | 43979 | 8:88449390 | 43993 | 8:88666966 |
| 44077 | 8:90752165 | 44176 | 8:93011122 | 44205 | 8:93906534 | 44262 | 8:95928376 | 44268 | 8:96045608 |
| 44269 | 8:96053216 | 44574 | 8:102774503 | 44648 | 8:104180690 | 44652 | 8:104213734 | 44749 | 8:106594158 |
| 44887 | 8:110915706 | 44899 | 8:111410824 | 45035 | 9:2664478 | 45036 | 9:2667437 | 45037 | 9:2675248 |
| 45038 | 9:2678406 | 45039 | 9:2679109 | 45040 | 9:2681750 | 45041 | 9:2685486 | 45079 | 9:3897414 |
| 45192 | 9:7600098 | 45344 | 9:10670291 | 45354 | 9:10721590 | 45379 | 9:10940149 | 45383 | 9:11053216 |
| 45423 | 9:12009299 | 45432 | 9:12198197 | 45496 | 9:13287682 | 45535 | 9:14277874 | 45589 | 9:15430684 |
| 45660 | 9:16629763 | 45662 | 9:16687402 | 45664 | 9:16727064 | 45676 | 9:17031856 | 45707 | 9:18068635 |
| 45744 | 9:19093497 | 45768 | 9:19999771 | 45832 | 9:21796887 | 45872 | 9:22692551 | 45957 | 9:24735769 |
| 45958 | 9:24739623 | 45959 | 9:24743503 | 45960 | 9:24744612 | 45961 | 9:24746634 | 45962 | 9:24747990 |
| 45963 | 9:24753059 | 45964 | 9:24757919 | 45965 | 9:24760506 | 45991 | 9:25685943 | 46046 | 9:27095343 |
| 46063 | 9:27473901 | 46065 | 9:27477523 | 46066 | 9:27485623 | 46191 | 9:29796827 | 46248 | 9:31114198 |
| 46259 | 9:31366005 | 46302 | 9:32301933 | 46386 | 9:34555386 | 46398 | 9:34906969 | 46462 | 9:36491907 |
| 46486 | 9:37002303 | 46488 | 9:37019786 | 46516 | 9:37778578 | 46518 | 9:37786775 | 46528 | 9:37990154 |
| 46609 | 9:39610171 | 46636 | 9:39977921 | 46841 | 9:45495778 | 46843 | 9:45524861 | 46886 | 9:46918750 |
| 46890 | 9:47015135 | 46938 | 9:48713344 | 46957 | 9:49281683 | 46958 | 9:49310703 | 46959 | 9:49332688 |
| 46969 | 9:49534025 | 46970 | 9:49560954 | 47019 | 9:50797460 | 47025 | 9:51023481 | 47032 | 9:51178639 |
| 47062 | 9:52118530 | 47097 | 9:52875308 | 47136 | 9:54016591 | 47139 | 9:54071333 | 47141 | 9:54121979 |
| 47143 | 9:54193058 | 47144 | 9:54241895 | 47147 | 9:54281836 | 47156 | 9:54592540 | 47165 | 9:54750120 |
| 47200 | 9:55772680 | 47203 | 9:55920760 | 47212 | 9:56203305 | 47237 | 9:56787230 | 47337 | 9:59589683 |
| 47486 | 9:63468566 | 47687 | 9:67996881 | 47691 | 9:68095521 | 47755 | 9:69703203 | 47782 | 9:70360272 |
| 47785 | 9:70387952 | 47920 | 9:73701336 | 48263 | 9:81076190 | 48268 | 9:81121582 | 48490 | 9:85784712 |
| 48495 | 9:85842378 | 48503 | 9:86006398 | 48511 | 9:86271158 | 48513 | 9:86326905 | 48558 | 9:87361936 |
| 48562 | 9:87394371 | 48620 | 9:88414100 | 48662 | 9:89255756 | 48663 | 9:89261575 | 48664 | 9:89266339 |
| 48691 | 9:89514561 | 48721 | 9:90252969 | 48732 | 9:90513278 | 48805 | 9:92056243 | 48810 | 9:92215180 |
| 48813 | 9:92280929 | 48924 | 9:94493145 | 49009 | 9:96178394 | 49069 | 9:96875482 | 49070 | 9:96875798 |
| 49073 | 9:96892725 | 49074 | 9:96894181 | 49096 | 9:97251687 | 49102 | 9:97327317 | 49126 | 9:97749602 |
| 49382 | 9:102242225 | 49467 | 9:103771813 | 49725 | 10:4803921 | 49789 | 10:5830788 | 49811 | 10:6389416 |
| 49838 | 10:6942796 | 49855 | 10:7506260 | 49870 | 10:7723009 | 49879 | 10:7896282 | 49918 | 10:8581573 |
| 49929 | 10:8932852 | 49954 | 10:9647790 | 49959 | 10:9706130 | 50090 | 10:12326160 | 50252 | 10:15438770 |
| 50302 | 10:16346333 | 50344 | 10:17006334 | 50394 | 10:18062302 | 50406 | 10:18136100 | 50425 | 10:18596826 |
| 50457 | 10:19155115 | 50490 | 10:19862667 | 50499 | 10:19959026 | 50578 | 10:21363314 | 50627 | 10:22355891 |
| 50628 | 10:22382830 | 50704 | 10:26294073 | 50713 | 10:26447113 | 50738 | 10:26790460 | 50793 | 10:27960775 |
| 50811 | 10:28566378 | 50812 | 10:28588817 | 50859 | 10:29085444 | 50868 | 10:29217264 | 50934 | 10:31208483 |
| 50952 | 10:31609187 | 50976 | 10:32324758 | 51059 | 10:34358150 | 51094 | 10:34985737 | 51138 | 10:35989426 |
| 51141 | 10:36035947 | 51147 | 10:36261135 | 51164 | 10:36819725 | 51193 | 10:37476784 | 51242 | 10:38651279 |
| 51294 | 10:40046559 | 51405 | 10:43227119 | 51426 | 10:43996215 | 51527 | 10:46095471 | 51600 | 10:47638179 |
| 51609 | 10:47789244 | 51610 | 10:47842702 | 51626 | 10:48320530 | 51629 | 10:48352886 | 51645 | 10:48589436 |
| 51649 | 10:48670398 | 51770 | 10:51376594 | 51773 | 10:51492328 | 51780 | 10:51664061 | 51801 | 10:52103291 |
| 51809 | 10:52286224 | 51824 | 10:52716176 | 51839 | 10:52867771 | 51896 | 10:54578642 | 51972 | 10:56658480 |
| 52166 | 10:62136409 | 52202 | 10:62942087 | 52272 | 10:64614710 | 52278 | 10:64660791 | 52409 | 10:67916863 |
| 52410 | 10:67917609 | 52411 | 10:67918635 | 52412 | 10:67919519 | 52416 | 10:68020797 | 52439 | 10:68405934 |
| 52471 | 10:69067745 | 52500 | 10:69834381 | 52520 | 10:70404549 | 52616 | 10:73493859 | 52730 | 10:76467129 |
| 52964 | 10:81617198 | 53012 | 10:82478545 | 53038 | 10:83193828 | 53039 | 10:83219578 | 53072 | 10:83953443 |
| 53105 | 10:84613620 | 53132 | 10:85160503 | 53157 | 10:85605717 | 53228 | 10:87305772 | 53332 | 10:89626819 |
| 53351 | 10:89936666 | 53372 | 10:90371369 | 53373 | 10:90396997 | 53380 | 10:90447669 | 53400 | 10:90743393 |
| 53411 | 10:90874981 | 53416 | 10:91003179 | 53424 | 10:91160509 | 53468 | 10:92328861 | 53547 | 10:94201919 |
| 53622 | 10:95898502 | 53688 | 10:97342537 | 53729 | 10:98302579 | 53731 | 10:98340013 | 53748 | 10:98561555 |
| 53769 | 10:99070199 | 53816 | 10:100101830 | 53837 | 10:100508994 | 53879 | 10:101228895 | 53880 | 10:101250880 |
| 53936 | 10:102180727 | 53960 | 10:102751503 | 54049 | 11:1244479 | 54111 | 11:1989902 | 54215 | 11:3345485 |
| 54221 | 11:3483025 | 54298 | 11:5089726 | 54369 | 11:6729148 | 54415 | 11:7795378 | 54432 | 11:8194895 |
| 54454 | 11:9031832 | 54487 | 11:9455596 | 54526 | 11:10334522 | 54533 | 11:10452880 | 54563 | 11:11221986 |
| 54593 | 11:11811943 | 54600 | 11:12001967 | 54611 | 11:12371073 | 54687 | 11:13859460 | 54718 | 11:15010708 |
| 54778 | 11:16210947 | 54799 | 11:16834406 | 54848 | 11:18344213 | 54849 | 11:18370765 | 54850 | 11:18396456 |
| 54959 | 11:21470638 | 55054 | 11:23592030 | 55056 | 11:23656903 | 55060 | 11:23740579 | 55083 | 11:24318038 |
| 55092 | 11:24369984 | 55093 | 11:24377983 | 55094 | 11:24393545 | 55096 | 11:24408850 | 55133 | 11:25341825 |
| 55231 | 11:27746425 | 55274 | 11:28736945 | 55276 | 11:28761952 | 55291 | 11:29024122 | 55369 | 11:30763811 |
| 55384 | 11:31041776 | 55392 | 11:31275179 | 55413 | 11:31882807 | 55416 | 11:31964009 | 55428 | 11:32373197 |
| 55474 | 11:33523211 | 55483 | 11:33718317 | 55559 | 11:36181120 | 55589 | 11:37017087 | 55594 | 11:37101530 |
| 55604 | 11:37283583 | 55776 | 11:41848496 | 55903 | 11:45028224 | 55916 | 11:45290515 | 55917 | 11:45313347 |
| 56006 | 11:47096563 | 56017 | 11:47326731 | 56096 | 11:48675652 | 56133 | 11:49165587 | 56145 | 11:49409414 |
| 56148 | 11:49467653 | 56177 | 11:50202868 | 56186 | 11:50607517 | 56242 | 11:52695227 | 56259 | 11:53270728 |
| 56262 | 11:53413882 | 56431 | 11:57988232 | 56468 | 11:58918358 | 56472 | 11:58971050 | 56480 | 11:59181535 |
| 56524 | 11:60468952 | 56540 | 11:60494769 | 56542 | 11:60517676 | 56544 | 11:60567347 | 56558 | 11:60775322 |
| 56559 | 11:60864552 | 56560 | 11:60906972 | 56594 | 11:61768776 | 56624 | 11:62610532 | 56627 | 11:62639185 |
| 56629 | 11:62703391 | 56635 | 11:62811507 | 56646 | 11:63105834 | 56648 | 11:63156170 | 56688 | 11:63881213 |
| 56694 | 11:63980697 | 56771 | 11:66096418 | 56774 | 11:66141532 | 56792 | 11:66500339 | 56805 | 11:66814255 |
| 56807 | 11:66889704 | 56808 | 11:66971573 | 56813 | 11:67084838 | 56826 | 11:67371438 | 56831 | 11:67445332 |
| 56833 | 11:67469876 | 56877 | 11:67856557 | 56883 | 11:68062927 | 56892 | 11:68380416 | 56894 | 11:68423346 |
| 56911 | 11:68884476 | 56915 | 11:68962608 | 56951 | 11:69554436 | 56976 | 11:70005013 | 56985 | 11:70216257 |
| 56988 | 11:70246998 | 56993 | 11:70300762 | 56996 | 11:70378153 | 57042 | 11:70810858 | 57106 | 11:72079051 |
| 57111 | 11:72169040 | 57219 | 11:74557125 | 57222 | 11:74693404 | 57231 | 11:75039779 | 57238 | 11:75245474 |
| 57239 | 11:75278281 | 57240 | 11:75301517 | 57244 | 11:75330473 | 57261 | 11:75610538 | 57269 | 11:75826371 |
| 57278 | 11:76006290 | 57282 | 11:76108140 | 57286 | 11:76291884 | 57299 | 11:76699098 | 57313 | 11:77009264 |
| 57332 | 11:77262392 | 57343 | 11:77494347 | 57355 | 11:77817486 | 57389 | 11:78010446 | 57391 | 11:78051004 |
| 57414 | 11:78619581 | 57476 | 11:79716380 | 57485 | 11:79945955 | 57486 | 11:80005355 | 57490 | 11:80160161 |
| 57509 | 11:80535106 | 57524 | 11:80835889 | 57534 | 11:81336077 | 57547 | 11:81697133 | 57619 | 11:82922365 |
| 57628 | 11:83118146 | 57667 | 11:84097016 | 57691 | 11:84931500 | 57733 | 11:85771451 | 57735 | 11:85810220 |
| 57756 | 11:86216179 | 57813 | 11:87691339 | 57824 | 11:87901219 | 57825 | 11:87933139 | 57838 | 11:87991576 |
| 57856 | 11:88008802 | 57871 | 11:88092604 | 57875 | 11:88114495 | 57883 | 11:88435971 | 57905 | 11:88999569 |
| 57918 | 11:89363137 | 58014 | 11:91483828 | 58015 | 11:91524490 | 58040 | 11:92016623 | 58199 | 11:95837864 |
| 58223 | 11:96479338 | 58254 | 11:97291509 | 58262 | 11:97559369 | 58265 | 11:97669268 | 58346 | 11:99292200 |
| 58369 | 11:99693004 | 58374 | 11:99779968 | 58381 | 11:99910936 | 58425 | 11:100730926 | 58480 | 11:101720696 |
| 58706 | 11:105729430 | 58710 | 11:105964095 | 58711 | 11:106026712 | 58887 | 12:3816364 | 58893 | 12:4017888 |
| 58924 | 12:4735467 | 59120 | 12:10585260 | 59121 | 12:10614578 | 59249 | 12:13606719 | 59254 | 12:13766685 |
| 59263 | 12:13857247 | 59320 | 12:15004714 | 59351 | 12:15565363 | 59376 | 12:15986894 | 59402 | 12:16562247 |
| 59446 | 12:17721084 | 59635 | 12:22358320 | 59648 | 12:22684761 | 59650 | 12:22780186 | 59656 | 12:22878113 |
| 59657 | 12:22955187 | 59658 | 12:22957971 | 59662 | 12:23024428 | 59686 | 12:23553969 | 59699 | 12:23968918 |
| 59725 | 12:24562389 | 59751 | 12:25043912 | 59818 | 12:26941585 | 59863 | 12:28021962 | 59864 | 12:28031415 |
| 59901 | 12:29149304 | 59931 | 12:30073919 | 59940 | 12:30237550 | 59970 | 12:30965466 | 59975 | 12:30995990 |
| 60105 | 12:34339978 | 60136 | 12:35202030 | 60146 | 12:35329106 | 60185 | 12:36506500 | 60202 | 12:37059151 |
| 60204 | 12:37117228 | 60206 | 12:37121439 | 60213 | 12:37336023 | 60264 | 12:39602051 | 60268 | 12:39726857 |
| 60368 | 12:43543060 | 60451 | 12:46609222 | 60452 | 12:46632594 | 60607 | 12:50328249 | 60608 | 12:50367458 |
| 60610 | 12:50405882 | 60655 | 12:51430846 | 60683 | 12:52226211 | 60688 | 12:52270911 | 60735 | 12:53308347 |
| 60768 | 12:53915464 | 60769 | 12:53924609 | 60780 | 12:54114793 | 60908 | 12:57839576 | 60967 | 12:59688270 |
| 61136 | 12:65124144 | 61209 | 12:67262559 | 61285 | 12:68831445 | 61316 | 12:69427741 | 61337 | 12:69681738 |
| 61357 | 12:70744278 | 61358 | 12:70747193 | 61411 | 12:72948314 | 61418 | 12:73037670 | 61419 | 12:73063509 |
| 61433 | 12:73378861 | 61437 | 12:73452468 | 61748 | 12:80191366 | 61809 | 12:81602505 | 61930 | 12:84189393 |
| 62050 | 12:86604613 | 62158 | 13:1818757 | 62173 | 13:2108292 | 62212 | 13:2694888 | 62217 | 13:2721397 |
| 62219 | 13:2775064 | 62220 | 13:2800282 | 62221 | 13:2813363 | 62257 | 13:3832665 | 62281 | 13:4414592 |
| 62310 | 13:5228105 | 62405 | 13:7418765 | 62519 | 13:10200560 | 62521 | 13:10297467 | 62539 | 13:10605573 |
| 62667 | 13:14736984 | 62723 | 13:16382635 | 62724 | 13:16385352 | 62728 | 13:16481332 | 62729 | 13:16481881 |
| 62730 | 13:16482962 | 62731 | 13:16484064 | 62732 | 13:16486013 | 62733 | 13:16487474 | 62734 | 13:16489553 |
| 62735 | 13:16494712 | 62736 | 13:16495172 | 62737 | 13:16495924 | 62738 | 13:16496828 | 62739 | 13:16497723 |
| 62740 | 13:16498500 | 62741 | 13:16499450 | 62742 | 13:16500106 | 62743 | 13:16505307 | 62774 | 13:17297996 |
| 62785 | 13:17376202 | 62832 | 13:18167455 | 62850 | 13:18708210 | 62851 | 13:18716183 | 62853 | 13:18721208 |
| 62854 | 13:18721251 | 62855 | 13:18723232 | 62856 | 13:18723968 | 62857 | 13:18724391 | 62858 | 13:18724682 |
| 62859 | 13:18724926 | 62860 | 13:18725319 | 62861 | 13:18725595 | 62862 | 13:18725670 | 62863 | 13:18725747 |
| 62864 | 13:18726353 | 62865 | 13:18727332 | 62866 | 13:18727382 | 62867 | 13:18728046 | 62868 | 13:18728216 |
| 62869 | 13:18729395 | 62870 | 13:18729428 | 62871 | 13:18729514 | 62872 | 13:18729597 | 62873 | 13:18732582 |
| 62874 | 13:18733271 | 62875 | 13:18733719 | 62876 | 13:18733742 | 62877 | 13:18733760 | 62878 | 13:18733884 |
| 62879 | 13:18735443 | 62882 | 13:18791943 | 62931 | 13:19690250 | 62932 | 13:19710676 | 62940 | 13:19814205 |
| 62956 | 13:20094078 | 62957 | 13:20180098 | 62960 | 13:20267627 | 62965 | 13:20376037 | 62975 | 13:20455892 |
| 62984 | 13:20631768 | 63067 | 13:22364650 | 63079 | 13:22622712 | 63125 | 13:24378395 | 63206 | 13:26030778 |
| 63286 | 13:28051297 | 63307 | 13:28631602 | 63318 | 13:28789636 | 63452 | 13:31297457 | 63463 | 13:31535167 |
| 63484 | 13:32188070 | 63485 | 13:32192701 | 63486 | 13:32195543 | 63488 | 13:32202239 | 63489 | 13:32206561 |
| 63490 | 13:32214896 | 63498 | 13:32363871 | 63515 | 13:32606184 | 63519 | 13:32649822 | 63534 | 13:32908367 |
| 63643 | 13:35552308 | 63655 | 13:35893139 | 63853 | 13:40115588 | 63937 | 13:42022321 | 63954 | 13:42455044 |
| 63957 | 13:42527459 | 63962 | 13:42563978 | 63996 | 13:42702842 | 64016 | 13:43224017 | 64031 | 13:43618688 |
| 64043 | 13:43868655 | 64084 | 13:44564660 | 64090 | 13:44735441 | 64093 | 13:44846012 | 64094 | 13:44877072 |
| 64095 | 13:44877728 | 64096 | 13:44883203 | 64097 | 13:44884376 | 64098 | 13:44891644 | 64099 | 13:44900803 |
| 64100 | 13:44901430 | 64101 | 13:44902570 | 64102 | 13:44908683 | 64104 | 13:44929091 | 64106 | 13:44941064 |
| 64107 | 13:44945347 | 64108 | 13:44954742 | 64109 | 13:44957480 | 64110 | 13:44959530 | 64111 | 13:44971418 |
| 64113 | 13:44974314 | 64114 | 13:44977218 | 64118 | 13:44984974 | 64120 | 13:44991424 | 64123 | 13:44994329 |
| 64124 | 13:44996032 | 64161 | 13:45580238 | 64220 | 13:46808920 | 64238 | 13:47284141 | 64266 | 13:47867949 |
| 64336 | 13:49568372 | 64337 | 13:49569350 | 64338 | 13:49570779 | 64339 | 13:49571658 | 64416 | 13:52005450 |
| 64426 | 13:52366895 | 64431 | 13:52518024 | 64456 | 13:52984857 | 64458 | 13:53006663 | 64466 | 13:53163449 |
| 64512 | 13:54065681 | 64629 | 13:57014498 | 64667 | 13:57988686 | 64672 | 13:58092691 | 64887 | 13:62737371 |
| 64900 | 13:62849693 | 64901 | 13:62871349 | 64910 | 13:63113668 | 64979 | 13:64765574 | 64992 | 13:65197131 |
| 65003 | 13:65421742 | 65046 | 13:66169238 | 65096 | 13:67321578 | 65143 | 13:68639312 | 65145 | 13:68664064 |
| 65159 | 13:69019407 | 65312 | 13:72652771 | 65332 | 13:73024654 | 65430 | 13:74713520 | 65439 | 13:75021624 |
| 65447 | 13:75318964 | 65448 | 13:75354084 | 65503 | 13:76376258 | 65507 | 13:76438492 | 65517 | 13:76641970 |
| 65529 | 13:76798256 | 65764 | 13:81126899 | 65905 | 14:1006940 | 65963 | 14:2212373 | 65993 | 14:3074708 |
| 65995 | 14:3098041 | 66091 | 14:4323988 | 66140 | 14:5386856 | 66177 | 14:5966351 | 66207 | 14:6303201 |
| 66289 | 14:7818027 | 66305 | 14:8003665 | 66326 | 14:8349092 | 66332 | 14:8441986 | 66350 | 14:8630299 |
| 66373 | 14:8996524 | 66493 | 14:11272085 | 66494 | 14:11275856 | 66495 | 14:11277881 | 66496 | 14:11278042 |
| 66497 | 14:11281126 | 66499 | 14:11294774 | 66500 | 14:11299050 | 66547 | 14:12222307 | 66586 | 14:12811226 |
| 66627 | 14:14043986 | 66687 | 14:15518676 | 66690 | 14:15530827 | 66691 | 14:15559743 | 66692 | 14:15601008 |
| 66693 | 14:15625272 | 66694 | 14:15626105 | 66695 | 14:15631968 | 66696 | 14:15643325 | 66698 | 14:15672366 |
| 66717 | 14:16009930 | 66718 | 14:16010978 | 66719 | 14:16022809 | 66724 | 14:16161546 | 66750 | 14:16670887 |
| 66775 | 14:17233811 | 66795 | 14:17760515 | 66799 | 14:17770830 | 66831 | 14:18480356 | 66832 | 14:18510376 |
| 66909 | 14:20574088 | 66969 | 14:21764414 | 66976 | 14:21914329 | 66987 | 14:22199112 | 66989 | 14:22291692 |
| 66991 | 14:22353534 | 66994 | 14:22402037 | 66997 | 14:22459009 | 66998 | 14:22460066 | 66999 | 14:22492158 |
| 67000 | 14:22499302 | 67001 | 14:22499882 | 67002 | 14:22500823 | 67003 | 14:22500981 | 67004 | 14:22501165 |
| 67005 | 14:22502609 | 67006 | 14:22503786 | 67007 | 14:22505668 | 67009 | 14:22551848 | 67010 | 14:22559672 |
| 67011 | 14:22559976 | 67012 | 14:22560498 | 67013 | 14:22560939 | 67014 | 14:22561597 | 67015 | 14:22562389 |
| 67016 | 14:22562808 | 67017 | 14:22562827 | 67018 | 14:22563098 | 67019 | 14:22563125 | 67020 | 14:22563136 |
| 67021 | 14:22563534 | 67022 | 14:22564168 | 67023 | 14:22564379 | 67024 | 14:22564874 | 67025 | 14:22565102 |
| 67026 | 14:22565724 | 67027 | 14:22565752 | 67028 | 14:22565896 | 67029 | 14:22565903 | 67030 | 14:22566273 |
| 67031 | 14:22566433 | 67032 | 14:22566621 | 67033 | 14:22567218 | 67034 | 14:22567252 | 67035 | 14:22567396 |
| 67036 | 14:22567418 | 67037 | 14:22567497 | 67038 | 14:22567570 | 67039 | 14:22568006 | 67040 | 14:22568446 |
| 67041 | 14:22568460 | 67042 | 14:22568517 | 67043 | 14:22568557 | 67044 | 14:22568700 | 67045 | 14:22568919 |
| 67046 | 14:22569314 | 67047 | 14:22569464 | 67048 | 14:22569577 | 67049 | 14:22569581 | 67050 | 14:22569980 |
| 67051 | 14:22570044 | 67052 | 14:22570058 | 67053 | 14:22570080 | 67054 | 14:22570301 | 67055 | 14:22570448 |
| 67056 | 14:22570466 | 67057 | 14:22570726 | 67058 | 14:22571422 | 67059 | 14:22573154 | 67060 | 14:22574799 |
| 67061 | 14:22579399 | 67062 | 14:22579809 | 67063 | 14:22581401 | 67064 | 14:22584062 | 67065 | 14:22584452 |
| 67066 | 14:22592312 | 67071 | 14:22671158 | 67072 | 14:22680795 | 67073 | 14:22681826 | 67079 | 14:22781305 |
| 67080 | 14:22830132 | 67081 | 14:22839488 | 67082 | 14:22841040 | 67083 | 14:22842180 | 67084 | 14:22861656 |
| 67085 | 14:22861757 | 67086 | 14:22861840 | 67087 | 14:22866278 | 67088 | 14:22867321 | 67089 | 14:22870443 |
| 67090 | 14:22872231 | 67091 | 14:22872707 | 67092 | 14:22875389 | 67094 | 14:22883080 | 67096 | 14:22887406 |
| 67097 | 14:22888105 | 67098 | 14:22895284 | 67099 | 14:22896212 | 67100 | 14:22899088 | 67101 | 14:22900130 |
| 67102 | 14:22900140 | 67103 | 14:22903945 | 67104 | 14:22904357 | 67105 | 14:22905670 | 67106 | 14:22915081 |
| 67107 | 14:22915304 | 67108 | 14:22916046 | 67109 | 14:22916365 | 67110 | 14:22917130 | 67111 | 14:22917282 |
| 67112 | 14:22918679 | 67113 | 14:22919099 | 67114 | 14:22922271 | 67115 | 14:22925305 | 67116 | 14:22926221 |
| 67117 | 14:22933624 | 67118 | 14:22935277 | 67119 | 14:22940329 | 67120 | 14:22941327 | 67121 | 14:22944551 |
| 67122 | 14:22945851 | 67123 | 14:22950339 | 67124 | 14:22951594 | 67125 | 14:22952187 | 67126 | 14:22952264 |
| 67127 | 14:22958318 | 67128 | 14:22959278 | 67129 | 14:22962984 | 67130 | 14:22963530 | 67131 | 14:22963552 |
| 67132 | 14:22963956 | 67133 | 14:22964530 | 67134 | 14:22968925 | 67135 | 14:22969225 | 67136 | 14:22969659 |
| 67137 | 14:22970008 | 67138 | 14:22983250 | 67139 | 14:22983836 | 67140 | 14:22986080 | 67141 | 14:22989108 |
| 67142 | 14:22989880 | 67143 | 14:22989917 | 67144 | 14:22995750 | 67145 | 14:22995861 | 67146 | 14:22999212 |
| 67147 | 14:22999475 | 67148 | 14:23000859 | 67149 | 14:23001109 | 67150 | 14:23001803 | 67151 | 14:23004072 |
| 67152 | 14:23005237 | 67153 | 14:23009266 | 67157 | 14:23168386 | 67175 | 14:23216693 | 67180 | 14:23217714 |
| 67181 | 14:23217718 | 67183 | 14:23219140 | 67189 | 14:23221714 | 67191 | 14:23232498 | 67193 | 14:23241336 |
| 67194 | 14:23244133 | 67195 | 14:23245996 | 67196 | 14:23246004 | 67197 | 14:23246030 | 67198 | 14:23246116 |
| 67199 | 14:23246121 | 67200 | 14:23246668 | 67201 | 14:23247018 | 67202 | 14:23247588 | 67203 | 14:23247661 |
| 67204 | 14:23247870 | 67205 | 14:23247878 | 67206 | 14:23247920 | 67207 | 14:23247940 | 67208 | 14:23248135 |
| 67209 | 14:23248201 | 67210 | 14:23248211 | 67211 | 14:23248448 | 67212 | 14:23248692 | 67213 | 14:23248770 |
| 67214 | 14:23248888 | 67215 | 14:23248943 | 67216 | 14:23249702 | 67217 | 14:23249738 | 67218 | 14:23249950 |
| 67219 | 14:23249976 | 67220 | 14:23250082 | 67221 | 14:23250315 | 67222 | 14:23250686 | 67223 | 14:23250690 |
| 67224 | 14:23250828 | 67225 | 14:23250871 | 67226 | 14:23250914 | 67227 | 14:23251020 | 67228 | 14:23251302 |
| 67229 | 14:23251581 | 67230 | 14:23251684 | 67231 | 14:23252354 | 67232 | 14:23252429 | 67233 | 14:23252563 |
| 67234 | 14:23252757 | 67235 | 14:23252809 | 67236 | 14:23253043 | 67237 | 14:23253210 | 67238 | 14:23253295 |
| 67239 | 14:23253300 | 67240 | 14:23253568 | 67241 | 14:23253746 | 67242 | 14:23253786 | 67243 | 14:23253805 |
| 67244 | 14:23254426 | 67245 | 14:23254453 | 67246 | 14:23254563 | 67247 | 14:23254639 | 67248 | 14:23254769 |
| 67249 | 14:23254807 | 67250 | 14:23254919 | 67251 | 14:23255007 | 67252 | 14:23255159 | 67253 | 14:23255587 |
| 67254 | 14:23255824 | 67255 | 14:23256710 | 67256 | 14:23257050 | 67257 | 14:23257092 | 67258 | 14:23257183 |
| 67259 | 14:23257229 | 67260 | 14:23257379 | 67261 | 14:23257441 | 67262 | 14:23257644 | 67263 | 14:23257665 |
| 67264 | 14:23257775 | 67265 | 14:23258452 | 67266 | 14:23258542 | 67267 | 14:23262536 | 67268 | 14:23262544 |
| 67269 | 14:23262984 | 67270 | 14:23264575 | 67271 | 14:23264736 | 67272 | 14:23265786 | 67273 | 14:23265991 |
| 67274 | 14:23266384 | 67275 | 14:23271813 | 67276 | 14:23273276 | 67277 | 14:23273801 | 67278 | 14:23274957 |
| 67279 | 14:23277049 | 67280 | 14:23278232 | 67281 | 14:23278652 | 67282 | 14:23278927 | 67283 | 14:23279593 |
| 67284 | 14:23279712 | 67285 | 14:23285130 | 67286 | 14:23288477 | 67287 | 14:23288921 | 67288 | 14:23289158 |
| 67289 | 14:23289589 | 67290 | 14:23289947 | 67291 | 14:23292612 | 67293 | 14:23296927 | 67294 | 14:23297204 |
| 67295 | 14:23298062 | 67296 | 14:23300304 | 67297 | 14:23314460 | 67298 | 14:23314730 | 67299 | 14:23314761 |
| 67300 | 14:23321577 | 67301 | 14:23326588 | 67302 | 14:23338890 | 67303 | 14:23343150 | 67304 | 14:23346065 |
| 67306 | 14:23383008 | 67308 | 14:23392938 | 67309 | 14:23408928 | 67310 | 14:23409204 | 67311 | 14:23409494 |
| 67312 | 14:23410644 | 67313 | 14:23410867 | 67314 | 14:23411712 | 67315 | 14:23414348 | 67316 | 14:23414443 |
| 67317 | 14:23421256 | 67318 | 14:23421807 | 67319 | 14:23422909 | 67320 | 14:23423768 | 67321 | 14:23423823 |
| 67322 | 14:23424726 | 67323 | 14:23426056 | 67324 | 14:23427548 | 67325 | 14:23429529 | 67326 | 14:23430628 |
| 67327 | 14:23430999 | 67328 | 14:23431167 | 67329 | 14:23438214 | 67330 | 14:23439941 | 67331 | 14:23440995 |
| 67332 | 14:23441483 | 67333 | 14:23443135 | 67334 | 14:23444334 | 67335 | 14:23446328 | 67336 | 14:23447775 |
| 67337 | 14:23450091 | 67338 | 14:23456836 | 67339 | 14:23458201 | 67340 | 14:23459603 | 67343 | 14:23528515 |
| 67344 | 14:23532117 | 67354 | 14:23600267 | 67355 | 14:23607944 | 67356 | 14:23622754 | 67357 | 14:23630896 |
| 67358 | 14:23639458 | 67359 | 14:23652804 | 67368 | 14:23825669 | 67370 | 14:23829057 | 67371 | 14:23829066 |
| 67372 | 14:23829235 | 67373 | 14:23830314 | 67375 | 14:23831223 | 67376 | 14:23831351 | 67377 | 14:23831451 |
| 67378 | 14:23831720 | 67379 | 14:23832062 | 67380 | 14:23832448 | 67381 | 14:23832632 | 67382 | 14:23832846 |
| 67383 | 14:23832941 | 67384 | 14:23833173 | 67385 | 14:23833835 | 67386 | 14:23834262 | 67387 | 14:23834735 |
| 67388 | 14:23834925 | 67389 | 14:23835136 | 67390 | 14:23835627 | 67391 | 14:23835870 | 67392 | 14:23835971 |
| 67393 | 14:23836131 | 67394 | 14:23836398 | 67395 | 14:23836443 | 67396 | 14:23836603 | 67397 | 14:23836691 |
| 67398 | 14:23836914 | 67399 | 14:23837002 | 67400 | 14:23838557 | 67401 | 14:23839177 | 67402 | 14:23840827 |
| 67403 | 14:23842863 | 67404 | 14:23848780 | 67405 | 14:23848797 | 67406 | 14:23848827 | 67407 | 14:23849503 |
| 67408 | 14:23852492 | 67409 | 14:23852815 | 67410 | 14:23852921 | 67411 | 14:23853184 | 67412 | 14:23853223 |
| 67413 | 14:23854487 | 67414 | 14:23856672 | 67415 | 14:23860303 | 67416 | 14:23862417 | 67418 | 14:23868149 |
| 67419 | 14:23882209 | 67420 | 14:23887365 | 67421 | 14:23887370 | 67422 | 14:23937950 | 67423 | 14:23945118 |
| 67425 | 14:23992830 | 67426 | 14:24019648 | 67430 | 14:24107226 | 67436 | 14:24297582 | 67440 | 14:24350585 |
| 67446 | 14:24477168 | 67447 | 14:24512384 | 67449 | 14:24516122 | 67450 | 14:24519148 | 67451 | 14:24519442 |
| 67452 | 14:24520182 | 67453 | 14:24522732 | 67454 | 14:24524138 | 67455 | 14:24527219 | 67456 | 14:24528754 |
| 67457 | 14:24532678 | 67458 | 14:24535392 | 67459 | 14:24536887 | 67460 | 14:24537213 | 67461 | 14:24537249 |
| 67462 | 14:24537652 | 67463 | 14:24537937 | 67464 | 14:24538192 | 67465 | 14:24538201 | 67466 | 14:24538525 |
| 67467 | 14:24538984 | 67468 | 14:24539180 | 67469 | 14:24539228 | 67470 | 14:24539315 | 67471 | 14:24539324 |
| 67472 | 14:24539556 | 67473 | 14:24539584 | 67474 | 14:24539696 | 67475 | 14:24540007 | 67476 | 14:24540057 |
| 67477 | 14:24540244 | 67478 | 14:24540270 | 67479 | 14:24542751 | 67481 | 14:24571024 | 67482 | 14:24580144 |
| 67483 | 14:24587368 | 67484 | 14:24594780 | 67485 | 14:24604764 | 67486 | 14:24609404 | 67487 | 14:24618912 |
| 67488 | 14:24644457 | 67493 | 14:24767026 | 67495 | 14:24790463 | 67496 | 14:24825146 | 67497 | 14:24859655 |
| 67500 | 14:24940746 | 67501 | 14:24941221 | 67502 | 14:24967239 | 67503 | 14:24967988 | 67504 | 14:24973247 |
| 67505 | 14:24978104 | 67506 | 14:24979482 | 67507 | 14:24982302 | 67508 | 14:24983741 | 67509 | 14:24986291 |
| 67510 | 14:25002052 | 67511 | 14:25030583 | 67513 | 14:25082860 | 67516 | 14:25152867 | 67517 | 14:25158355 |
| 67526 | 14:25266063 | 67530 | 14:25440652 | 67533 | 14:25548588 | 67534 | 14:25548611 | 67535 | 14:25548858 |
| 67536 | 14:25549944 | 67537 | 14:25550026 | 67538 | 14:25550835 | 67539 | 14:25550879 | 67540 | 14:25551206 |
| 67541 | 14:25551423 | 67542 | 14:25551661 | 67543 | 14:25551868 | 67544 | 14:25552256 | 67545 | 14:25552486 |
| 67546 | 14:25552782 | 67547 | 14:25552984 | 67548 | 14:25553034 | 67549 | 14:25553066 | 67550 | 14:25553217 |
| 67552 | 14:25555831 | 67613 | 14:26283998 | 67621 | 14:26472341 | 67627 | 14:26644064 | 67628 | 14:26666519 |
| 67686 | 14:28503857 | 67690 | 14:28611968 | 67696 | 14:28706164 | 67714 | 14:29246244 | 67717 | 14:29321450 |
| 67723 | 14:29455092 | 67743 | 14:29818291 | 67756 | 14:30089537 | 67838 | 14:32001174 | 67864 | 14:32341495 |
| 67882 | 14:32807225 | 67883 | 14:32884654 | 67887 | 14:32989933 | 67910 | 14:33408077 | 67918 | 14:33505061 |
| 67920 | 14:33551756 | 67973 | 14:34950627 | 67979 | 14:35239973 | 67980 | 14:35240016 | 67981 | 14:35240021 |
| 67982 | 14:35240029 | 67983 | 14:35240427 | 67984 | 14:35240523 | 67985 | 14:35240587 | 67986 | 14:35240759 |
| 67987 | 14:35240947 | 67988 | 14:35241115 | 67989 | 14:35241328 | 67990 | 14:35241474 | 67991 | 14:35241672 |
| 67992 | 14:35265340 | 67998 | 14:35425257 | 68003 | 14:35595531 | 68006 | 14:35751572 | 68010 | 14:35894072 |
| 68024 | 14:36171691 | 68025 | 14:36225604 | 68027 | 14:36267667 | 68045 | 14:36581802 | 68070 | 14:37486005 |
| 68085 | 14:37852302 | 68092 | 14:38020858 | 68098 | 14:38241778 | 68104 | 14:38511542 | 68107 | 14:38549650 |
| 68121 | 14:38865578 | 68136 | 14:39381019 | 68138 | 14:39446327 | 68154 | 14:39861013 | 68181 | 14:40565849 |
| 68184 | 14:40702265 | 68188 | 14:40809782 | 68195 | 14:41149021 | 68202 | 14:41340996 | 68219 | 14:41739836 |
| 68239 | 14:42191184 | 68242 | 14:42286395 | 68265 | 14:42846993 | 68302 | 14:44079419 | 68338 | 14:44933472 |
| 68350 | 14:45112392 | 68358 | 14:45283009 | 68368 | 14:45610432 | 68374 | 14:45681264 | 68376 | 14:45772293 |
| 68391 | 14:46030748 | 68402 | 14:46236143 | 68403 | 14:46236637 | 68404 | 14:46237110 | 68405 | 14:46238761 |
| 68406 | 14:46241868 | 68407 | 14:46244088 | 68408 | 14:46250918 | 68418 | 14:46418593 | 68424 | 14:46603107 |
| 68441 | 14:46995812 | 68444 | 14:47059409 | 68474 | 14:48033192 | 68483 | 14:48202902 | 68512 | 14:48835114 |
| 68819 | 14:56925358 | 68973 | 14:60573159 | 68974 | 14:60591022 | 69042 | 14:62002265 | 69055 | 14:62159723 |
| 69056 | 14:62160097 | 69093 | 14:62960532 | 69145 | 14:64219535 | 69158 | 14:64538337 | 69236 | 14:66565905 |
| 69296 | 14:67751963 | 69405 | 14:70557126 | 69479 | 14:72621724 | 69527 | 14:73767156 | 69546 | 14:74251050 |
| 69666 | 14:77010786 | 69676 | 14:77348388 | 69701 | 14:77989698 | 69824 | 14:80695704 | 69841 | 14:81015684 |
| 70102 | 15:4692832 | 70287 | 15:5550484 | 70295 | 15:5724091 | 70403 | 15:8239471 | 70559 | 15:13400749 |
| 70578 | 15:14002939 | 70584 | 15:14158577 | 70619 | 15:14996109 | 70634 | 15:15286305 | 70711 | 15:16549253 |
| 70719 | 15:16763241 | 70746 | 15:17193061 | 70781 | 15:18160360 | 70783 | 15:18238844 | 70920 | 15:21418237 |
| 70933 | 15:21792771 | 70955 | 15:22295223 | 71033 | 15:23794449 | 71075 | 15:24300165 | 71076 | 15:24314377 |
| 71084 | 15:24406959 | 71092 | 15:24625723 | 71106 | 15:24813538 | 71225 | 15:27497541 | 71246 | 15:28011102 |
| 71266 | 15:28279946 | 71274 | 15:28453282 | 71275 | 15:28497953 | 71313 | 15:28951920 | 71373 | 15:30506918 |
| 71421 | 15:31626657 | 71443 | 15:32064162 | 71459 | 15:32448502 | 71467 | 15:32684505 | 71489 | 15:33066384 |
| 71494 | 15:33140066 | 71500 | 15:33283643 | 71516 | 15:33757310 | 71519 | 15:33784264 | 71542 | 15:34144843 |
| 71544 | 15:34205212 | 71548 | 15:34249244 | 71550 | 15:34273013 | 71565 | 15:34545054 | 71594 | 15:35124174 |
| 71637 | 15:36171696 | 71643 | 15:36281231 | 71644 | 15:36316689 | 71668 | 15:36835278 | 71670 | 15:36913421 |
| 71721 | 15:38013431 | 71735 | 15:38345972 | 71814 | 15:40225597 | 71892 | 15:41883212 | 71918 | 15:42285508 |
| 71919 | 15:42310362 | 71922 | 15:42461633 | 71961 | 15:43061218 | 71966 | 15:43113772 | 71983 | 15:43322372 |
| 72013 | 15:43808690 | 72034 | 15:44213989 | 72038 | 15:44321644 | 72039 | 15:44348210 | 72041 | 15:44377304 |
| 72043 | 15:44404296 | 72081 | 15:45199864 | 72107 | 15:45767348 | 72150 | 15:46515608 | 72163 | 15:46717734 |
| 72166 | 15:46782723 | 72172 | 15:46863275 | 72181 | 15:47142381 | 72226 | 15:48040954 | 72245 | 15:48275779 |
| 72278 | 15:49421011 | 72280 | 15:49469161 | 72294 | 15:49994423 | 72324 | 15:51010384 | 72368 | 15:51564179 |
| 72387 | 15:51844292 | 72392 | 15:51981969 | 72395 | 15:51992306 | 72396 | 15:52000306 | 72403 | 15:52160095 |
| 72434 | 15:52969357 | 72473 | 15:53341502 | 72559 | 15:55149996 | 72592 | 15:56124060 | 72635 | 15:56767431 |
| 72636 | 15:56774921 | 72637 | 15:56780183 | 72638 | 15:56782530 | 72639 | 15:56783614 | 72640 | 15:56784736 |
| 72641 | 15:56785971 | 72642 | 15:56797332 | 72643 | 15:56807110 | 72646 | 15:56815479 | 72647 | 15:56818346 |
| 72648 | 15:56829119 | 72649 | 15:56831561 | 72650 | 15:56832894 | 72651 | 15:56833644 | 72652 | 15:56834347 |
| 72653 | 15:56835434 | 72654 | 15:56836055 | 72655 | 15:56836949 | 72656 | 15:56838230 | 72657 | 15:56838871 |
| 72658 | 15:56840204 | 72659 | 15:56841525 | 72660 | 15:56842815 | 72661 | 15:56843668 | 72662 | 15:56845155 |
| 72663 | 15:56851874 | 72664 | 15:56853748 | 72665 | 15:56855128 | 72666 | 15:56856657 | 72667 | 15:56868653 |
| 72668 | 15:56869977 | 72690 | 15:57484879 | 72695 | 15:57536250 | 72767 | 15:58882110 | 72769 | 15:58979088 |
| 72784 | 15:59472006 | 72833 | 15:60847989 | 72869 | 15:61639615 | 72884 | 15:61810673 | 72900 | 15:62235431 |
| 72906 | 15:62456943 | 72909 | 15:62484563 | 72910 | 15:62511268 | 72918 | 15:62618258 | 72922 | 15:62694367 |
| 72976 | 15:63833091 | 72988 | 15:64092090 | 73022 | 15:64696473 | 73048 | 15:65009502 | 73052 | 15:65067229 |
| 73130 | 15:66545124 | 73132 | 15:66614036 | 73460 | 15:75001650 | 73465 | 15:75074384 | 73560 | 15:77284842 |
| 73564 | 15:77342823 | 73599 | 15:77992295 | 73602 | 15:78001169 | 73610 | 15:78017138 | 73626 | 15:78135787 |
| 73719 | 15:80696386 | 73731 | 15:81021484 | 73760 | 15:81377169 | 73768 | 15:81639702 | 73794 | 15:82390046 |
| 73813 | 15:82751747 | 73814 | 15:82766106 | 73826 | 15:82951372 | 73834 | 15:83031817 | 73835 | 15:83043883 |
| 73836 | 15:83046338 | 73837 | 15:83054032 | 73838 | 15:83058015 | 73839 | 15:83063624 | 73945 | 16:1306469 |
| 73949 | 16:1350556 | 73955 | 16:1432322 | 74009 | 16:2521425 | 74012 | 16:2584565 | 74027 | 16:3043683 |
| 74058 | 16:3453206 | 74076 | 16:3954831 | 74079 | 16:3986796 | 74218 | 16:7674017 | 74248 | 16:8845857 |
| 74278 | 16:9938750 | 74286 | 16:10168137 | 74356 | 16:12347316 | 74366 | 16:12489625 | 74382 | 16:12870890 |
| 74389 | 16:12974308 | 74390 | 16:12974917 | 74391 | 16:12975122 | 74392 | 16:12975220 | 74393 | 16:12975427 |
| 74394 | 16:12975489 | 74395 | 16:12975573 | 74396 | 16:12975644 | 74460 | 16:14726498 | 74462 | 16:14841844 |
| 74520 | 16:16433701 | 74547 | 16:17397768 | 74579 | 16:18551432 | 74583 | 16:18633047 | 74592 | 16:18835344 |
| 74621 | 16:19568001 | 74641 | 16:20043212 | 74669 | 16:20811063 | 74702 | 16:21584184 | 74853 | 16:25049887 |
| 74963 | 16:26927766 | 74986 | 16:27682201 | 75041 | 16:29174251 | 75060 | 16:29760720 | 75062 | 16:29811081 |
| 75073 | 16:30136403 | 75075 | 16:30161226 | 75076 | 16:30185523 | 75134 | 16:31704359 | 75143 | 16:31896105 |
| 75150 | 16:32035645 | 75163 | 16:32472255 | 75166 | 16:32570779 | 75268 | 16:35045999 | 75271 | 16:35171790 |
| 75403 | 16:38151804 | 75444 | 16:39447477 | 75514 | 16:41852637 | 75544 | 16:41964938 | 75601 | 16:44301023 |
| 75602 | 16:44309062 | 75603 | 16:44341029 | 75675 | 16:46932908 | 75678 | 16:47002315 | 75805 | 16:49746045 |
| 75821 | 16:50190328 | 75822 | 16:50240719 | 75839 | 16:50745473 | 75941 | 16:53280547 | 75975 | 16:54055090 |
| 75976 | 16:54077469 | 75987 | 16:54386293 | 76037 | 16:55777077 | 76379 | 16:63448062 | 76420 | 16:64372019 |
| 76474 | 16:65163914 | 76705 | 16:70129856 | 76767 | 16:71527211 | 76903 | 16:74041271 | 76913 | 16:74276107 |
| 76922 | 16:74520515 | 76932 | 16:74777059 | 76992 | 16:75746134 | 77018 | 16:76272938 | 77019 | 16:76273016 |
| 77054 | 16:77113967 | 77055 | 16:77114869 | 77056 | 16:77115406 | 77057 | 16:77115489 | 77058 | 16:77115511 |
| 77059 | 16:77115516 | 77060 | 16:77117453 | 77061 | 16:77124198 | 77227 | 16:80002018 | 77342 | 17:2538151 |
| 77417 | 17:4163909 | 77468 | 17:5077558 | 77480 | 17:5375823 | 77508 | 17:5999177 | 77613 | 17:8012177 |
| 77632 | 17:8453908 | 77860 | 17:14044398 | 77915 | 17:14966600 | 78029 | 17:17475831 | 78037 | 17:17737256 |
| 78038 | 17:17740826 | 78097 | 17:19033632 | 78335 | 17:26269335 | 78410 | 17:28015501 | 78497 | 17:30487913 |
| 78560 | 17:31986086 | 78574 | 17:32403842 | 78580 | 17:32489547 | 78638 | 17:33714773 | 78647 | 17:33972684 |
| 78649 | 17:34035563 | 78655 | 17:34228544 | 78680 | 17:34921588 | 78708 | 17:35827456 | 78896 | 17:40827993 |
| 79097 | 17:45794152 | 79104 | 17:45901126 | 79186 | 17:47856402 | 79401 | 17:52535678 | 79405 | 17:52588133 |
| 79527 | 17:55767778 | 79528 | 17:55769246 | 79534 | 17:55892881 | 79577 | 17:57167393 | 79582 | 17:57279391 |
| 79748 | 17:60357076 | 79754 | 17:60395994 | 79778 | 17:60781502 | 79835 | 17:61664121 | 79837 | 17:61736121 |
| 79860 | 17:62385783 | 79871 | 17:62699773 | 79873 | 17:62731132 | 79876 | 17:62794522 | 79877 | 17:62820495 |
| 79916 | 17:63612425 | 79928 | 17:63638557 | 79962 | 17:64318575 | 80008 | 17:65293792 | 80030 | 17:65746912 |
| 80050 | 17:66155014 | 80061 | 17:66243332 | 80076 | 17:66473136 | 80079 | 17:66581559 | 80083 | 17:66695254 |
| 80109 | 17:67239268 | 80249 | 17:70356568 | 80285 | 17:70987613 | 80303 | 17:71333851 | 80363 | 17:72400135 |
| 80592 | 18:3769740 | 80636 | 18:4682446 | 80731 | 18:6411919 | 80773 | 18:7301014 | 80800 | 18:7879780 |
| 80807 | 18:8137616 | 80811 | 18:8163590 | 80812 | 18:8166965 | 80813 | 18:8169505 | 80848 | 18:8910517 |
| 80881 | 18:9393342 | 80887 | 18:9441291 | 80933 | 18:10341158 | 80973 | 18:11082461 | 81013 | 18:11950846 |
| 81055 | 18:13195401 | 81339 | 18:19844264 | 81450 | 18:22200958 | 81455 | 18:22265614 | 81495 | 18:23178646 |
| 81501 | 18:23288724 | 81645 | 18:26295597 | 81707 | 18:27873728 | 81901 | 18:33007379 | 81923 | 18:33448672 |
| 81932 | 18:33625498 | 81933 | 18:33654608 | 81936 | 18:33679712 | 81937 | 18:33689480 | 81939 | 18:33731026 |
| 81940 | 18:33765822 | 81945 | 18:33836053 | 81946 | 18:33868226 | 82061 | 18:36768945 | 82065 | 18:36877351 |
| 82083 | 18:37328492 | 82099 | 18:37566458 | 82114 | 18:37873077 | 82177 | 18:39306524 | 82178 | 18:39344592 |
| 82179 | 18:39385561 | 82181 | 18:39440689 | 82184 | 18:39467461 | 82186 | 18:39488321 | 82190 | 18:39592965 |
| 82370 | 18:43157279 | 82379 | 18:43316323 | 82382 | 18:43368967 | 82508 | 18:46424023 | 82580 | 18:46784427 |
| 82583 | 18:46865748 | 82670 | 18:48351248 | 82698 | 18:49004338 | 82732 | 18:49949153 | 82736 | 18:49996660 |
| 82831 | 18:52094939 | 82855 | 18:52727408 | 82885 | 18:53596284 | 82887 | 18:53619711 | 82909 | 18:53938427 |
| 82919 | 18:54093736 | 82940 | 18:54546398 | 82942 | 18:54586408 | 82983 | 18:55141854 | 82984 | 18:55173246 |
| 83013 | 18:55944026 | 83110 | 18:57018026 | 83153 | 18:57798250 | 83172 | 18:57998293 | 83257 | 18:59897080 |
| 83291 | 18:61008032 | 83319 | 18:61724391 | 83595 | 19:414615 | 84130 | 19:13223983 | 84131 | 19:13225470 |
| 84175 | 19:13777565 | 84315 | 19:16698614 | 84335 | 19:16990785 | 84387 | 19:17914841 | 84393 | 19:18036338 |
| 84409 | 19:18449930 | 84457 | 19:19539554 | 84577 | 19:21388744 | 84702 | 19:23904908 | 84703 | 19:23933777 |
| 84742 | 19:24383348 | 84781 | 19:24846129 | 84846 | 19:25706233 | 85007 | 19:28109982 | 85011 | 19:28147842 |
| 85061 | 19:29186854 | 85119 | 19:30424898 | 85120 | 19:30433795 | 85121 | 19:30435544 | 85178 | 19:31844296 |
| 85211 | 19:32510963 | 85345 | 19:34890238 | 85357 | 19:34981210 | 85366 | 19:35152019 | 85409 | 19:36144917 |
| 85481 | 19:37101024 | 85602 | 19:40338519 | 85855 | 19:45407828 | 85921 | 19:46516531 | 85941 | 19:47101703 |
| 85951 | 19:47151917 | 85971 | 19:47631810 | 86015 | 19:48407091 | 86087 | 19:49926869 | 86124 | 19:50694652 |
| 86143 | 19:50789408 | 86202 | 19:51325894 | 86259 | 19:52650212 | 86283 | 19:53097970 | 86292 | 19:53198253 |
| 86293 | 19:53206247 | 86319 | 19:53762930 | 86348 | 19:54137367 | 86384 | 19:54758347 | 86416 | 19:55474861 |
| 86488 | 19:56006886 | 86522 | 19:56138971 | 86531 | 19:56253321 | 86568 | 19:56726108 | 86580 | 19:56980273 |
| 86813 | 19:61050416 | 86822 | 19:61175458 | 86893 | 19:62160575 | 86898 | 19:62249612 | 86900 | 19:62272214 |
| 86997 | 20:909954 | 87043 | 20:1738655 | 87052 | 20:1886807 | 87083 | 20:2362867 | 87101 | 20:2682912 |
| 87142 | 20:3442790 | 87149 | 20:3585646 | 87174 | 20:4263007 | 87176 | 20:4284310 | 87177 | 20:4331799 |
| 87190 | 20:4516902 | 87193 | 20:4587717 | 87194 | 20:4587833 | 87195 | 20:4587839 | 87196 | 20:4588506 |
| 87197 | 20:4588549 | 87198 | 20:4588618 | 87199 | 20:4588637 | 87200 | 20:4588654 | 87203 | 20:4620871 |
| 87204 | 20:4621075 | 87205 | 20:4621110 | 87206 | 20:4621122 | 87207 | 20:4622072 | 87208 | 20:4622166 |
| 87209 | 20:4622378 | 87210 | 20:4622780 | 87211 | 20:4622804 | 87212 | 20:4622902 | 87213 | 20:4623044 |
| 87214 | 20:4623114 | 87215 | 20:4623572 | 87216 | 20:4624342 | 87217 | 20:4624492 | 87218 | 20:4624645 |
| 87219 | 20:4625064 | 87220 | 20:4625438 | 87221 | 20:4626150 | 87222 | 20:4626194 | 87223 | 20:4626939 |
| 87224 | 20:4627062 | 87225 | 20:4628179 | 87226 | 20:4628239 | 87227 | 20:4629196 | 87228 | 20:4629784 |
| 87229 | 20:4630247 | 87230 | 20:4630301 | 87231 | 20:4630500 | 87232 | 20:4630563 | 87233 | 20:4630761 |
| 87234 | 20:4630879 | 87235 | 20:4630925 | 87236 | 20:4631015 | 87237 | 20:4631031 | 87238 | 20:4631157 |
| 87239 | 20:4631655 | 87240 | 20:4632251 | 87241 | 20:4632285 | 87242 | 20:4632908 | 87243 | 20:4633050 |
| 87244 | 20:4633087 | 87245 | 20:4633727 | 87246 | 20:4633738 | 87247 | 20:4633830 | 87248 | 20:4634166 |
| 87249 | 20:4634320 | 87250 | 20:4634619 | 87251 | 20:4634657 | 87252 | 20:4635386 | 87253 | 20:4635483 |
| 87254 | 20:4635990 | 87255 | 20:4637075 | 87256 | 20:4637125 | 87257 | 20:4638775 | 87258 | 20:4638910 |
| 87259 | 20:4639096 | 87260 | 20:4640118 | 87261 | 20:4640234 | 87262 | 20:4644059 | 87263 | 20:4644327 |
| 87264 | 20:4652157 | 87265 | 20:4654656 | 87267 | 20:4658089 | 87268 | 20:4658953 | 87269 | 20:4661324 |
| 87270 | 20:4665088 | 87271 | 20:4666775 | 87272 | 20:4667789 | 87275 | 20:4685771 | 87277 | 20:4690335 |
| 87278 | 20:4690908 | 87279 | 20:4691249 | 87280 | 20:4697066 | 87281 | 20:4697631 | 87282 | 20:4701728 |
| 87283 | 20:4701832 | 87284 | 20:4708012 | 87285 | 20:4709731 | 87286 | 20:4710087 | 87287 | 20:4712587 |
| 87288 | 20:4713160 | 87289 | 20:4715176 | 87290 | 20:4716043 | 87291 | 20:4726582 | 87292 | 20:4728514 |
| 87293 | 20:4730765 | 87294 | 20:4732330 | 87296 | 20:4736819 | 87297 | 20:4741050 | 87298 | 20:4750279 |
| 87299 | 20:4751162 | 87300 | 20:4752621 | 87301 | 20:4753160 | 87302 | 20:4757934 | 87303 | 20:4758722 |
| 87304 | 20:4761361 | 87305 | 20:4763955 | 87306 | 20:4770162 | 87309 | 20:4797785 | 87310 | 20:4805282 |
| 87311 | 20:4812157 | 87312 | 20:4819449 | 87314 | 20:4838678 | 87315 | 20:4841899 | 87316 | 20:4842019 |
| 87317 | 20:4846348 | 87318 | 20:4850816 | 87319 | 20:4851612 | 87320 | 20:4853215 | 87321 | 20:4854589 |
| 87322 | 20:4863651 | 87323 | 20:4873619 | 87324 | 20:4873645 | 87325 | 20:4873658 | 87326 | 20:4873667 |
| 87327 | 20:4880129 | 87328 | 20:4885163 | 87329 | 20:4922714 | 87330 | 20:4927232 | 87332 | 20:4928741 |
| 87333 | 20:4936772 | 87334 | 20:4937498 | 87335 | 20:4938011 | 87336 | 20:4938521 | 87337 | 20:4938780 |
| 87338 | 20:4938885 | 87339 | 20:4939294 | 87340 | 20:4940782 | 87341 | 20:4940805 | 87342 | 20:4944507 |
| 87343 | 20:4947601 | 87344 | 20:4949204 | 87345 | 20:4964757 | 87346 | 20:4965179 | 87347 | 20:4965760 |
| 87348 | 20:4969010 | 87349 | 20:4971347 | 87350 | 20:4971831 | 87351 | 20:4972329 | 87352 | 20:4974179 |
| 87354 | 20:4975337 | 87355 | 20:4978069 | 87356 | 20:4978213 | 87357 | 20:4978475 | 87359 | 20:4982228 |
| 87360 | 20:4984846 | 87361 | 20:4985523 | 87362 | 20:4986398 | 87363 | 20:4988142 | 87364 | 20:4990340 |
| 87365 | 20:4991225 | 87367 | 20:4992704 | 87368 | 20:4992712 | 87369 | 20:4993558 | 87370 | 20:4993669 |
| 87371 | 20:4993957 | 87373 | 20:4999299 | 87374 | 20:4999988 | 87376 | 20:5002715 | 87377 | 20:5003850 |
| 87378 | 20:5004638 | 87379 | 20:5008738 | 87380 | 20:5009598 | 87383 | 20:5040971 | 87385 | 20:5045805 |
| 87386 | 20:5049196 | 87387 | 20:5054905 | 87388 | 20:5055091 | 87389 | 20:5055857 | 87390 | 20:5058209 |
| 87391 | 20:5070761 | 87392 | 20:5074751 | 87393 | 20:5075106 | 87394 | 20:5076823 | 87395 | 20:5079687 |
| 87397 | 20:5081368 | 87399 | 20:5082509 | 87400 | 20:5107041 | 87402 | 20:5129728 | 87415 | 20:5282517 |
| 87429 | 20:5485968 | 87434 | 20:5633232 | 87435 | 20:5665364 | 87436 | 20:5695030 | 87438 | 20:5754805 |
| 87439 | 20:5796361 | 87449 | 20:6060819 | 87450 | 20:6062848 | 87451 | 20:6063007 | 87452 | 20:6063251 |
| 87453 | 20:6063553 | 87454 | 20:6063862 | 87455 | 20:6063984 | 87456 | 20:6064358 | 87457 | 20:6064442 |
| 87458 | 20:6066220 | 87459 | 20:6066792 | 87460 | 20:6066880 | 87461 | 20:6067197 | 87462 | 20:6069267 |
| 87463 | 20:6069326 | 87464 | 20:6069531 | 87465 | 20:6072588 | 87466 | 20:6073779 | 87467 | 20:6076915 |
| 87470 | 20:6090071 | 87477 | 20:6208301 | 87484 | 20:6325657 | 87488 | 20:6444472 | 87490 | 20:6526713 |
| 87509 | 20:6914755 | 87513 | 20:7017878 | 87516 | 20:7049088 | 87519 | 20:7079494 | 87521 | 20:7089351 |
| 87537 | 20:7437979 | 87539 | 20:7473329 | 87548 | 20:7706288 | 87550 | 20:7739561 | 87557 | 20:7958728 |
| 87559 | 20:7978859 | 87564 | 20:8099584 | 87566 | 20:8120294 | 87570 | 20:8190372 | 87573 | 20:8244125 |
| 87599 | 20:8976316 | 87608 | 20:9074647 | 87609 | 20:9107005 | 87662 | 20:10266518 | 87686 | 20:10946439 |
| 87698 | 20:11213830 | 87702 | 20:11274635 | 87711 | 20:11438125 | 87734 | 20:11948110 | 87736 | 20:12001911 |
| 87747 | 20:12228546 | 87750 | 20:12265622 | 87755 | 20:12409400 | 87758 | 20:12445726 | 87824 | 20:13931338 |
| 87826 | 20:13967565 | 87829 | 20:14050805 | 87830 | 20:14120849 | 87885 | 20:16058541 | 87923 | 20:17088438 |
| 87931 | 20:17251657 | 88103 | 20:21950333 | 88188 | 20:22481257 | 88190 | 20:22578241 | 88220 | 20:23180410 |
| 88233 | 20:23492042 | 88239 | 20:23575761 | 88241 | 20:23623587 | 88244 | 20:23695033 | 88334 | 20:25681663 |
| 88342 | 20:26011480 | 88352 | 20:26319951 | 88372 | 20:26721455 | 88379 | 20:26929602 | 88486 | 20:29337360 |
| 88501 | 20:29797692 | 88594 | 20:32271969 | 88629 | 20:33323766 | 88635 | 20:33477656 | 88636 | 20:33482995 |
| 88651 | 20:33788019 | 88843 | 20:38077292 | 88880 | 20:39076620 | 88893 | 20:39383171 | 88900 | 20:39458208 |
| 88901 | 20:39462501 | 88903 | 20:39469722 | 88906 | 20:39485628 | 88970 | 20:41005245 | 88987 | 20:41215061 |
| 89024 | 20:41990636 | 89201 | 20:46912182 | 89292 | 20:49691751 | 89327 | 20:50794831 | 89331 | 20:50908612 |
| 89334 | 20:50953384 | 89388 | 20:52906490 | 89424 | 20:53680094 | 89427 | 20:53779182 | 89490 | 20:55580535 |
| 89491 | 20:55615423 | 89501 | 20:55920256 | 89504 | 20:55944017 | 89603 | 20:58030736 | 89607 | 20:58078386 |
| 89748 | 20:61558977 | 89850 | 20:63825165 | 89852 | 20:63847390 | 89855 | 20:63882214 | 89947 | 20:65988032 |
| 89964 | 20:66468697 | 90010 | 20:67443129 | 90042 | 20:68014966 | 90105 | 20:69097447 | 90110 | 20:69174801 |
| 90114 | 20:69288227 | 90116 | 20:69317277 | 90161 | 20:70296851 | 90314 | 21:2109801 | 90321 | 21:2416354 |
| 90340 | 21:2937636 | 90422 | 21:5168513 | 90441 | 21:5776117 | 90447 | 21:5802937 | 90449 | 21:5865604 |
| 90452 | 21:5932063 | 90531 | 21:8044281 | 90549 | 21:8125084 | 90577 | 21:8739099 | 90673 | 21:10967078 |
| 90676 | 21:11012534 | 90870 | 21:15628004 | 90871 | 21:15629471 | 90872 | 21:15634222 | 90873 | 21:15635039 |
| 90874 | 21:15636617 | 90875 | 21:15640534 | 90876 | 21:15644415 | 90877 | 21:15648341 | 90878 | 21:15652428 |
| 90879 | 21:15656729 | 90964 | 21:17632245 | 90983 | 21:18074742 | 90987 | 21:18139041 | 91008 | 21:18709801 |
| 91009 | 21:18745959 | 91041 | 21:19240261 | 91119 | 21:20568083 | 91133 | 21:20667605 | 91163 | 21:21254778 |
| 91164 | 21:21278489 | 91244 | 21:22980503 | 91310 | 21:24894924 | 91377 | 21:26762300 | 91417 | 21:27993786 |
| 91435 | 21:28714736 | 91624 | 21:33373608 | 91661 | 21:33774652 | 91702 | 21:34416654 | 91768 | 21:35722374 |
| 91868 | 21:38504846 | 91877 | 21:38644972 | 91941 | 21:40541468 | 91985 | 21:41630642 | 92011 | 21:42193358 |
| 92020 | 21:42461759 | 92029 | 21:42678313 | 92115 | 21:44563811 | 92120 | 21:44677322 | 92171 | 21:46302745 |
| 92332 | 21:49387921 | 92335 | 21:49487176 | 92397 | 21:51289914 | 92407 | 21:51606765 | 92484 | 21:53761679 |
| 92488 | 21:53814553 | 92726 | 21:57261794 | 92848 | 21:59894217 | 92855 | 21:59981922 | 92967 | 21:62224560 |
| 93037 | 21:64128429 | 93155 | 21:66126088 | 93201 | 21:67026936 | 93296 | 21:69054783 | 93298 | 21:69111628 |
| 93301 | 21:69165607 | 93601 | 22:5786344 | 93653 | 22:6683071 | 93663 | 22:6874059 | 93668 | 22:6973667 |
| 93683 | 22:7347580 | 93785 | 22:10006376 | 93807 | 22:10285746 | 93819 | 22:10597656 | 93821 | 22:10648705 |
| 93829 | 22:10879669 | 93832 | 22:10961055 | 93894 | 22:12251220 | 94047 | 22:16130993 | 94110 | 22:17624753 |
| 94177 | 22:19212118 | 94255 | 22:21157473 | 94279 | 22:21651340 | 94283 | 22:21690013 | 94326 | 22:22930737 |
| 94380 | 22:24606457 | 94410 | 22:25532201 | 94504 | 22:28088504 | 94526 | 22:28449795 | 94614 | 22:30572708 |
| 94616 | 22:30617079 | 94621 | 22:30762175 | 94689 | 22:32709942 | 94691 | 22:32740432 | 94806 | 22:35660903 |
| 94908 | 22:38005262 | 94968 | 22:39268641 | 95000 | 22:39706378 | 95013 | 22:39906000 | 95037 | 22:40607655 |
| 95117 | 22:42731135 | 95121 | 22:42898400 | 95122 | 22:42936001 | 95284 | 22:46414503 | 95300 | 22:46810720 |
| 95307 | 22:46910055 | 95353 | 22:47726147 | 95583 | 22:53279742 | 95622 | 22:54013319 | 95666 | 22:54873498 |
| 95669 | 22:54947766 | 95676 | 22:55073957 | 95716 | 22:55626326 | 95781 | 22:56400223 | 95784 | 22:56416067 |
| 95785 | 22:56419658 | 95786 | 22:56420213 | 95809 | 22:56829490 | 95908 | 22:58476458 | 95913 | 22:58524949 |
| 95955 | 22:59413554 | 96087 | 23:2001057 | 96090 | 23:2028074 | 96118 | 23:2653886 | 96121 | 23:2672734 |
| 96124 | 23:2696666 | 96171 | 23:3661084 | 96177 | 23:3744075 | 96232 | 23:4789896 | 96422 | 23:8682465 |
| 96482 | 23:10137524 | 96546 | 23:11326784 | 96550 | 23:11418685 | 96552 | 23:11493234 | 96644 | 23:13147408 |
| 96647 | 23:13234890 | 96777 | 23:15576268 | 96872 | 23:17807855 | 96930 | 23:19250498 | 96931 | 23:19271779 |
| 96933 | 23:19307959 | 96941 | 23:19459539 | 96947 | 23:19483200 | 96959 | 23:19635143 | 96965 | 23:19711944 |
| 96996 | 23:20201294 | 97043 | 23:21249255 | 97051 | 23:21366692 | 97071 | 23:21956033 | 97096 | 23:22583782 |
| 97278 | 23:25346058 | 97357 | 23:27009758 | 97451 | 23:27999156 | 97501 | 23:28242781 | 97780 | 23:32969917 |
| 97828 | 23:34109165 | 97943 | 23:37547849 | 97981 | 23:38637301 | 98382 | 23:45418705 | 98389 | 23:45533685 |
| 98404 | 23:46021030 | 98438 | 23:46778964 | 98556 | 23:48912747 | 98631 | 23:50033982 | 98724 | 23:51773706 |
| 98783 | 24:520222 | 98832 | 24:1526726 | 98846 | 24:1793795 | 98892 | 24:2708333 | 98894 | 24:2766149 |
| 98913 | 24:3184889 | 98915 | 24:3208218 | 98935 | 24:3712541 | 98952 | 24:4048939 | 98964 | 24:4188152 |
| 98982 | 24:4612976 | 98994 | 24:5077959 | 99037 | 24:6230000 | 99125 | 24:8320822 | 99297 | 24:10714691 |
| 99353 | 24:12020111 | 99356 | 24:12116867 | 99361 | 24:12381665 | 99369 | 24:12616739 | 99402 | 24:13417942 |
| 99414 | 24:13675366 | 99451 | 24:14795404 | 99594 | 24:19151288 | 99665 | 24:20730048 | 99691 | 24:21214702 |
| 99863 | 24:25023821 | 99870 | 24:25259209 | 99898 | 24:25782545 | 99909 | 24:25992482 | 100009 | 24:28570245 |
| 100053 | 24:29754026 | 100059 | 24:29814575 | 100120 | 24:31415877 | 100199 | 24:32894188 | 100245 | 24:34122422 |
| 100257 | 24:34395443 | 100267 | 24:34719362 | 100285 | 24:35185903 | 100316 | 24:35736460 | 100323 | 24:35860154 |
| 100338 | 24:36195505 | 100358 | 24:37011897 | 100362 | 24:37166678 | 100364 | 24:37203930 | 100429 | 24:39199046 |
| 100782 | 24:46297155 | 100791 | 24:46409035 | 100919 | 24:49840655 | 100982 | 24:51488828 | 101005 | 24:51881818 |
| 101010 | 24:51956661 | 101014 | 24:52037619 | 101245 | 24:57052356 | 101347 | 24:59016719 | 101349 | 24:59106967 |
| 101394 | 24:60056629 | 101395 | 24:60093951 | 101469 | 24:61534805 | 101551 | 25:1163011 | 101605 | 25:1918071 |
| 101614 | 25:2105032 | 101618 | 25:2179579 | 101802 | 25:3997589 | 101849 | 25:4921820 | 101868 | 25:5339582 |
| 101958 | 25:6766372 | 101966 | 25:6952022 | 102005 | 25:7896632 | 102011 | 25:7949848 | 102018 | 25:8118349 |
| 102131 | 25:10832103 | 102149 | 25:11113745 | 102307 | 25:14593246 | 102318 | 25:14917112 | 102323 | 25:15012071 |
| 102329 | 25:15106926 | 102356 | 25:15663604 | 102365 | 25:15912161 | 102380 | 25:16148690 | 102399 | 25:16456322 |
| 102413 | 25:16841496 | 102459 | 25:17915319 | 102460 | 25:17952726 | 102462 | 25:18009869 | 102490 | 25:18825304 |
| 102523 | 25:19553139 | 102596 | 25:21400170 | 102612 | 25:21756847 | 102647 | 25:22463379 | 102656 | 25:22697194 |
| 102676 | 25:23209925 | 102679 | 25:23393931 | 102695 | 25:23751702 | 102713 | 25:24073747 | 102724 | 25:24300333 |
| 102732 | 25:24496560 | 102767 | 25:25119747 | 102774 | 25:25175370 | 102851 | 25:26611224 | 102875 | 25:27289405 |
| 102971 | 25:29170307 | 102973 | 25:29196073 | 102976 | 25:29227741 | 103031 | 25:30198863 | 103036 | 25:30292715 |
| 103045 | 25:30543167 | 103047 | 25:30590381 | 103165 | 25:33056358 | 103226 | 25:34314708 | 103243 | 25:34536825 |
| 103265 | 25:34878010 | 103273 | 25:35037480 | 103294 | 25:35416991 | 103369 | 25:36281889 | 103468 | 25:37785020 |
| 103522 | 25:38925016 | 103562 | 25:39799663 | 103598 | 25:40282487 | 103603 | 25:40362153 | 103698 | 25:42167568 |
| 103761 | 26:1999995 | 103766 | 26:2270743 | 103811 | 26:2874049 | 103964 | 26:7323964 | 104015 | 26:8514472 |
| 104061 | 26:9801561 | 104175 | 26:12542210 | 104214 | 26:13232302 | 104243 | 26:13935413 | 104244 | 26:13974845 |
| 104248 | 26:14058998 | 104265 | 26:14560437 | 104266 | 26:14562388 | 104420 | 26:18423545 | 104469 | 26:19395441 |
| 104485 | 26:19632302 | 104679 | 26:24855737 | 104714 | 26:25459976 | 104715 | 26:25463718 | 104716 | 26:25465826 |
| 104717 | 26:25467173 | 104781 | 26:26561752 | 104860 | 26:28504622 | 104925 | 26:30397842 | 104932 | 26:30643098 |
| 104935 | 26:30753391 | 104947 | 26:31085230 | 105378 | 26:41389219 | 105381 | 26:41451309 | 105394 | 26:41900876 |
| 105445 | 26:42982393 | 105499 | 26:44238502 | 105742 | 26:49454109 | 105754 | 26:49637614 | 105813 | 26:51001537 |
| 105824 | 26:51205579 | 105851 | 26:51911589 | 105854 | 26:51990348 | 105886 | 27:1292211 | 105932 | 27:2099758 |
| 105955 | 27:2545985 | 106036 | 27:4477596 | 106056 | 27:5041534 | 106064 | 27:5224821 | 106086 | 27:5489950 |
| 106115 | 27:6134492 | 106135 | 27:7463182 | 106203 | 27:8803096 | 106277 | 27:10171403 | 106312 | 27:11344637 |
| 106334 | 27:11976000 | 106374 | 27:12998392 | 106452 | 27:13945896 | 106510 | 27:15278766 | 106547 | 27:16013203 |
| 106553 | 27:16091265 | 106558 | 27:16161541 | 106608 | 27:16989882 | 106617 | 27:17229318 | 106655 | 27:18039702 |
| 106668 | 27:18444933 | 106672 | 27:18549440 | 106674 | 27:18561932 | 106695 | 27:18999723 | 106753 | 27:20126660 |
| 106800 | 27:21693284 | 106837 | 27:22606696 | 106839 | 27:22612038 | 106840 | 27:22625012 | 106841 | 27:22635635 |
| 106851 | 27:22872901 | 106892 | 27:23844184 | 106958 | 27:25097722 | 106963 | 27:25227473 | 107031 | 27:26703643 |
| 107032 | 27:26707702 | 107033 | 27:26709108 | 107034 | 27:26717112 | 107035 | 27:26752145 | 107037 | 27:26821155 |
| 107116 | 27:28602334 | 107142 | 27:29184261 | 107215 | 27:30821123 | 107219 | 27:30967912 | 107275 | 27:31920386 |
| 107418 | 27:35019984 | 107424 | 27:35045715 | 107434 | 27:35072059 | 107439 | 27:35098802 | 107452 | 27:35316815 |
| 107478 | 27:35710124 | 107512 | 27:36366267 | 107541 | 27:37168180 | 107629 | 27:38769358 | 107630 | 27:38784049 |
| 107632 | 27:38825952 | 107648 | 27:39079734 | 107650 | 27:39140091 | 107659 | 27:39414442 | 107684 | 27:40138920 |
| 107723 | 27:41177956 | 107855 | 27:44120630 | 107940 | 28:1026816 | 107943 | 28:1063432 | 107978 | 28:1669722 |
| 107979 | 28:1676667 | 107980 | 28:1688640 | 108035 | 28:3310204 | 108064 | 28:4807450 | 108072 | 28:5021515 |
| 108093 | 28:5430772 | 108094 | 28:5471888 | 108109 | 28:5837959 | 108120 | 28:5926093 | 108142 | 28:6534383 |
| 108248 | 28:9052050 | 108503 | 28:15552135 | 108505 | 28:15620196 | 108621 | 28:17961688 | 108634 | 28:18086730 |
| 108720 | 28:20471303 | 108761 | 28:21757902 | 108775 | 28:22091224 | 108882 | 28:24568402 | 108911 | 28:25196334 |
| 108950 | 28:26054541 | 108997 | 28:26764566 | 109005 | 28:26976598 | 109043 | 28:27784145 | 109225 | 28:31516271 |
| 109243 | 28:31960964 | 109334 | 28:33369552 | 109355 | 28:33823084 | 109363 | 28:34002468 | 109397 | 28:34663948 |
| 109420 | 28:34999208 | 109504 | 28:37068693 | 109511 | 28:37195142 | 109515 | 28:37311281 | 109519 | 28:37446774 |
| 109549 | 28:37921033 | 109557 | 28:38098897 | 109621 | 28:39341830 | 109741 | 28:40986070 | 109744 | 28:41014073 |
| 109881 | 28:43434313 | 109929 | 28:44311425 | 109935 | 28:44453574 | 109945 | 28:44780535 | 109958 | 28:44981684 |
| 109988 | 28:45527255 | 110035 | 29:1014606 | 110080 | 29:1997329 | 110082 | 29:2117573 | 110221 | 29:5006464 |
| 110233 | 29:5358686 | 110234 | 29:5358699 | 110248 | 29:5885429 | 110252 | 29:5979872 | 110429 | 29:10496324 |
| 110449 | 29:11034651 | 110590 | 29:14470262 | 110606 | 29:14866248 | 110721 | 29:17901471 | 110722 | 29:17951025 |
| 110723 | 29:17955354 | 110769 | 29:19855058 | 110770 | 29:19861520 | 110771 | 29:19862538 | 110985 | 29:25169363 |
| 110987 | 29:25193462 | 111011 | 29:25759092 | 111063 | 29:27485292 | 111143 | 29:28949555 | 111155 | 29:29347332 |
| 111211 | 29:30136039 | 111212 | 29:30157180 | 111221 | 29:30347114 | 111336 | 29:32940189 | 111340 | 29:33039863 |
| 111384 | 29:34046874 | 111390 | 29:34239637 | 111402 | 29:34523872 | 111422 | 29:34808241 | 111479 | 29:35951280 |
| 111481 | 29:35999607 | 111550 | 29:37304798 | 111551 | 29:37320062 | 111658 | 29:40811556 | 111659 | 29:40812741 |
| 111660 | 29:40813920 | 111661 | 29:40818497 | 111662 | 29:40820427 | 111663 | 29:40820963 | 111664 | 29:40821065 |
| 111666 | 29:40825872 | 111806 | 29:43407527 | 111808 | 29:43424442 | 111892 | 29:44721351 | 111901 | 29:45041058 |
| 111904 | 29:45172298 | 111928 | 29:45712362 | 111979 | 29:46833503 | 111989 | 29:46997866 | 111995 | 29:47125684 |
| 111996 | 29:47150455 | 112100 | 29:49234278 | 112124 | 29:49722188 | 112140 | 29:50217955 | 112141 | 29:50260533 |

In the present disclosure, a sixth marker panel was preferably obtained from two current mainstream commercial beadchips to obtain effective information SNPs of Huaxi cattle, and an obtaining method preferably included: genotyping results of Illumina BovineHD beadchip and Neogen GGP Bovine 100K beadchip in Huaxi cattle population were allowed to have quality control at a MAF of greater than 0.1, a genotype deletion rate of less than 10%, and a P value with HWE testing of 1×10⁻⁶, while a total of 74,098 effective information markers were retained in Huaxi cattle population.

In the present disclosure, a method for obtaining a seventh marker panel preferably included: the markers derived from the above six marker panels were summarized, and a total of 109,563 variations of the ARS-UCD 1.2/bosTau9 genome version were obtained by deduplication of biostatistical methods. In order to ensure that the markers were evenly distributed on the chromosome, the gap regions of the genome larger than 100 kb were filled. The sources of filling SNPs were preferably prioritized as follows: Illumina BovineHD beadchip, whole-genome resequencing data, and Neogen GGP Bovine 100K beadchip. Considering the MAF value and a distance from the SNP locus in each gap to both ends, a total of 2,617 SNPs for gap filling with uniform distribution of chromosomes were finally obtained and added to the chip. The information of seventh marker panel was shown in Table 6:

TABLE 6
Location information of seventh marker panel

| NO. | Position | NO | Position | NO. | Position | NO. | Position | NO. | Position |
|---|---|---|---|---|---|---|---|---|---|
| 1 | 1:198611 | 2 | 1:354105 | 3 | 1:413686 | 4 | 1:503436 | 5 | 1:539006 |
| 6 | 1:561519 | 8 | 1:752778 | 114 | 1:2548682 | 115 | 1:2645413 | 116 | 1:2738165 |
| 120 | 1:2859590 | 248 | 1:5217586 | 249 | 1:5308367 | 394 | 1:8387543 | 518 | 1:11192831 |
| 542 | 1:11918795 | 549 | 1:12187898 | 551 | 1:12295655 | 578 | 1:13034308 | 581 | 1:13135427 |
| 624 | 1:13936005 | 625 | 1:14033343 | 634 | 1:14325639 | 689 | 1:15749427 | 709 | 1:16317871 |
| 793 | 1:19108624 | 795 | 1:19246215 | 816 | 1:19761054 | 817 | 1:19840115 | 906 | 1:22225114 |
| 926 | 1:22716338 | 927 | 1:22813570 | 942 | 1:23156851 | 943 | 1:23255421 | 950 | 1:23587167 |
| 1081 | 1:27110041 | 1096 | 1:27690352 | 1099 | 1:27826641 | 1121 | 1:28708529 | 1184 | 1:30341410 |
| 1188 | 1:30639309 | 1228 | 1:31833724 | 1232 | 1:32038632 | 1239 | 1:32383066 | 1245 | 1:32604779 |
| 1302 | 1:34248126 | 1303 | 1:34341221 | 1336 | 1:35240562 | 1357 | 1:35661327 | 1373 | 1:36152445 |
| 1491 | 1:39146665 | 1500 | 1:39465349 | 1501 | 1:39555945 | 1528 | 1:40302747 | 1529 | 1:40392357 |
| 1555 | 1:41086735 | 1563 | 1:41385809 | 1565 | 1:41492237 | 1626 | 1:42693702 | 1627 | 1:42705366 |
| 1628 | 1:42797258 | 1769 | 1:45607475 | 1771 | 1:45687245 | 1772 | 1:45787243 | 1818 | 1:47287965 |
| 1838 | 1:47969845 | 1845 | 1:48250419 | 1876 | 1:49256468 | 1899 | 1:50064432 | 1921 | 1:50739583 |
| 1956 | 1:51942050 | 1961 | 1:52098640 | 2014 | 1:53514840 | 2090 | 1:54855775 | 2101 | 1:55175239 |
| 2117 | 1:55765709 | 2124 | 1:55975682 | 2125 | 1:56071064 | 2227 | 1:58399681 | 2228 | 1:58497604 |
| 2264 | 1:59389492 | 2269 | 1:59650598 | 2357 | 1:61707467 | 2364 | 1:61908265 | 2533 | 1:65639354 |
| 2534 | 1:65691897 | 2542 | 1:65975858 | 2553 | 1:66261981 | 2554 | 1:66349787 | 2575 | 1:66970472 |
| 2601 | 1:67667419 | 2612 | 1:68106244 | 2693 | 1:69418925 | 2702 | 1:69664149 | 2804 | 1:71695300 |
| 2825 | 1:72263210 | 2865 | 1:73046338 | 2912 | 1:74307455 | 2913 | 1:74402077 | 2942 | 1:75146396 |
| 2992 | 1:76510120 | 3001 | 1:76727873 | 3014 | 1:77141699 | 3091 | 1:78826134 | 3171 | 1:80384212 |
| 3239 | 1:81998130 | 3282 | 1:82911267 | 3295 | 1:83439908 | 3307 | 1:83899382 | 3321 | 1:84316332 |
| 3356 | 1:85642906 | 3372 | 1:86015500 | 3389 | 1:86437496 | 3390 | 1:86526242 | 3403 | 1:86930423 |
| 3415 | 1:87291852 | 3447 | 1:88278205 | 3448 | 1:88303482 | 3449 | 1:88477613 | 3450 | 1:88510142 |
| 3451 | 1:88605108 | 3480 | 1:89106677 | 3481 | 1:89184242 | 3605 | 1:92127457 | 3615 | 1:92303587 |
| 3627 | 1:92748568 | 3631 | 1:92911886 | 3662 | 1:93749721 | 3788 | 1:96724055 | 3898 | 1:99648631 |
| 3899 | 1:99745058 | 3907 | 1:99964213 | 3912 | 1:100151147 | 3951 | 1:101402516 | 3959 | 1:101604960 |
| 3975 | 1:102178820 | 3985 | 1:102499753 | 4011 | 1:103210827 | 4043 | 1:104445688 | 4047 | 1:104601851 |
| 4128 | 1:106679632 | 4215 | 1:108995880 | 4218 | 1:109138566 | 4223 | 1:109344061 | 4244 | 1:109812035 |
| 4303 | 1:110891497 | 4316 | 1:111431902 | 4391 | 1:113109748 | 4461 | 1:114671853 | 4507 | 1:116130150 |
| 4509 | 1:116269280 | 4510 | 1:116368715 | 4688 | 1:119719715 | 4739 | 1:120951015 | 4741 | 1:121018439 |
| 4742 | 1:121074170 | 4743 | 1:121144456 | 4745 | 1:121224156 | 4749 | 1:121384190 | 4759 | 1:121640449 |
| 4764 | 1:121788170 | 4774 | 1:122051101 | 4832 | 1:123574290 | 4833 | 1:123657947 | 4976 | 1:126688131 |
| 4977 | 1:126726689 | 4978 | 1:126823765 | 5040 | 1:128486623 | 5043 | 1:128606933 | 5093 | 1:129682985 |
| 5098 | 1:129814612 | 5099 | 1:129913741 | 5241 | 1:132429392 | 5247 | 1:132635025 | 5251 | 1:132828896 |
| 5260 | 1:133234769 | 5284 | 1:134043255 | 5414 | 1:136299174 | 5432 | 1:136970680 | 5444 | 1:137364879 |
| 5459 | 1:138082957 | 5460 | 1:138180043 | 5462 | 1:138259482 | 5464 | 1:138404453 | 5500 | 1:139245663 |
| 5502 | 1:139304863 | 5503 | 1:139403568 | 5511 | 1:139653452 | 5578 | 1:141255563 | 5701 | 1:143320922 |
| 5754 | 1:144299047 | 5884 | 1:146674083 | 5885 | 1:146773640 | 6509 | 1:157687863 | 6510 | 1:157775988 |
| 6512 | 1:157881491 | 6523 | 1:158316722 | 6524 | 1:158361834 | 6525 | 1:158460274 | 6527 | 2:97987 |
| 6528 | 2:156099 | 6529 | 2:251841 | 6609 | 2:1676588 | 6610 | 2:1762272 | 6627 | 2:2208427 |
| 6711 | 2:4459297 | 6819 | 2:6376890 | 6897 | 2:7947030 | 6898 | 2:7984204 | 6899 | 2:8067862 |
| 6921 | 2:8713754 | 6943 | 2:9417024 | 6945 | 2:9540631 | 6961 | 2:9879504 | 6962 | 2:9977673 |
| 6990 | 2:10739801 | 6994 | 2:11002563 | 7030 | 2:12006500 | 7035 | 2:12186267 | 7046 | 2:12679896 |
| 7117 | 2:14600625 | 7167 | 2:15807478 | 7249 | 2:17949516 | 7391 | 2:20586534 | 7405 | 2:20982246 |
| 7482 | 2:22953223 | 7490 | 2:23131851 | 7539 | 2:24040103 | 7590 | 2:25076321 | 7805 | 2:29677272 |
| 7836 | 2:30463341 | 7892 | 2:31678013 | 7912 | 2:32367398 | 7928 | 2:32964948 | 8001 | 2:34879480 |
| 8002 | 2:34940227 | 8003 | 2:35037207 | 8015 | 2:35478468 | 8021 | 2:35685945 | 8062 | 2:36835120 |
| 8185 | 2:40374618 | 8195 | 2:40590316 | 8364 | 2:44741204 | 8381 | 2:45256936 | 8524 | 2:48700283 |
| 8548 | 2:49196955 | 8550 | 2:49308768 | 8552 | 2:49502134 | 8577 | 2:50260674 | 8621 | 2:51619705 |
| 8622 | 2:51714357 | 8648 | 2:52249299 | 8665 | 2:52682136 | 8707 | 2:53776420 | 8717 | 2:54059294 |
| 8723 | 2:54299723 | 8817 | 2:56671164 | 8818 | 2:56766656 | 8859 | 2:57934056 | 8886 | 2:58700978 |
| 8907 | 2:59199942 | 8956 | 2:60394889 | 8972 | 2:60962014 | 9010 | 2:61641610 | 9011 | 2:61738618 |
| 9018 | 2:62012037 | 9023 | 2:62250012 | 9101 | 2:64316198 | 9116 | 2:64570288 | 9246 | 2:68272329 |
| 9258 | 2:68690400 | 9261 | 2:68901070 | 9264 | 2:69034698 | 9269 | 2:69214365 | 9274 | 2:69379425 |
| 9323 | 2:70760400 | 9362 | 2:71684921 | 9363 | 2:71782525 | 9365 | 2:71848257 | 9367 | 2:71970045 |
| 9406 | 2:73054461 | 9417 | 2:73255223 | 9483 | 2:75058828 | 9582 | 2:77785420 | 9651 | 2:79212662 |
| 9652 | 2:79232935 | 9655 | 2:79435179 | 9688 | 2:80269896 | 9691 | 2:80350552 | 9692 | 2:80443906 |
| 9718 | 2:81298125 | 9721 | 2:81552356 | 9731 | 2:82160162 | 9738 | 2:82525466 | 9741 | 2:82703708 |
| 9757 | 2:83398924 | 9759 | 2:83488349 | 9790 | 2:84070506 | 9793 | 2:84186911 | 9820 | 2:84735988 |
| 9822 | 2:84748768 | 9823 | 2:84845633 | 9848 | 2:85539387 | 9873 | 2:86113417 | 9878 | 2:86175227 |
| 9879 | 2:86265979 | 9908 | 2:86950234 | 9953 | 2:88328898 | 9980 | 2:88998896 | 10067 | 2:90047120 |
| 10069 | 2:90182742 | 10070 | 2:90271127 | 10071 | 2:90346277 | 10107 | 2:91439705 | 10170 | 2:92960490 |
| 10178 | 2:93253701 | 10243 | 2:94504077 | 10356 | 2:96858239 | 10362 | 2:97119985 | 10364 | 2:97284470 |
| 10374 | 2:97660903 | 10388 | 2:97949623 | 10453 | 2:99661757 | 10479 | 2:100148811 | 10510 | 2:101083832 |
| 10528 | 2:101669777 | 10534 | 2:101822460 | 10562 | 2:102640055 | 10642 | 2:104771582 | 10671 | 2:105313365 |
| 10916 | 2:110949976 | 10930 | 2:111452102 | 11063 | 2:114575713 | 11083 | 2:115163293 | 11148 | 2:116629100 |
| 11191 | 2:117806168 | 11194 | 2:117939332 | 11231 | 2:118820218 | 11232 | 2:118916665 | 11274 | 2:119675734 |
| 11302 | 2:120419474 | 11330 | 2:121151600 | 11342 | 2:121489660 | 11349 | 2:121623817 | 11350 | 2:121707578 |
| 11363 | 2:122105560 | 11448 | 2:124381727 | 11449 | 2:124471534 | 11500 | 2:125909343 | 11658 | 2:128226965 |
| 11765 | 2:130549076 | 11809 | 2:131471648 | 12088 | 2:135958653 | 12089 | 2:136042952 | 12096 | 2:136122518 |
| 12097 | 2:136218516 | 12103 | 3:192288 | 12172 | 3:1379812 | 12218 | 3:2245775 | 12219 | 3:2335779 |
| 12428 | 3:6422650 | 12571 | 3:8746180 | 12573 | 3:8864644 | 12579 | 3:9185006 | 12699 | 3:10683176 |
| 12753 | 3:11779967 | 12757 | 3:11849178 | 12758 | 3:11934240 | 12759 | 3:11998748 | 12811 | 3:13516552 |
| 12962 | 3:16327244 | 13006 | 3:17313416 | 13007 | 3:17408532 | 13036 | 3:18384479 | 13087 | 3:19384406 |
| 13088 | 3:19480721 | 13103 | 3:19938418 | 13104 | 3:19975811 | 13105 | 3:20062027 | 13114 | 3:20219541 |
| 13115 | 3:20275444 | 13134 | 3:20949426 | 13135 | 3:21041525 | 13139 | 3:21260248 | 13153 | 3:21701072 |
| 13164 | 3:21935930 | 13203 | 3:22637339 | 13253 | 3:23859633 | 13388 | 3:26747082 | 13484 | 3:28324475 |
| 13485 | 3:28415348 | 13486 | 3:28426219 | 13487 | 3:28519784 | 13535 | 3:29477079 | 14013 | 3:38769939 |
| 14015 | 3:38908661 | 14018 | 3:39010474 | 14019 | 3:39102130 | 14042 | 3:39825859 | 14046 | 3:39962964 |
| 14061 | 3:40312602 | 14077 | 3:40804040 | 14079 | 3:40921947 | 14112 | 3:41900576 | 14171 | 3:43138350 |
| 14186 | 3:43521434 | 14223 | 3:44490132 | 14224 | 3:44561235 | 14250 | 3:45348826 | 14257 | 3:45588541 |
| 14268 | 3:45973111 | 14270 | 3:46106986 | 14307 | 3:47132786 | 14310 | 3:47350830 | 14401 | 3:49233756 |
| 14490 | 3:50263076 | 14511 | 3:50885592 | 14519 | 3:51298193 | 14525 | 3:51591043 | 14548 | 3:52087305 |
| 14576 | 3:52675804 | 14622 | 3:53900489 | 14624 | 3:54093636 | 14635 | 3:54224439 | 14636 | 3:54256413 |
| 14637 | 3:54344299 | 14639 | 3:54400470 | 14640 | 3:54475982 | 14641 | 3:54564882 | 14642 | 3:54618300 |
| 14643 | 3:54697466 | 14644 | 3:54793255 | 14645 | 3:54888122 | 14679 | 3:55617345 | 14680 | 3:55712074 |
| 14682 | 3:55757845 | 14683 | 3:55857361 | 14699 | 3:56411615 | 14703 | 3:56614407 | 14724 | 3:57120778 |
| 14725 | 3:57210410 | 14730 | 3:57316623 | 14763 | 3:58252296 | 14807 | 3:59522050 | 14820 | 3:59954303 |
| 14878 | 3:61139535 | 14937 | 3:62995597 | 14986 | 3:64406567 | 15080 | 3:66682938 | 15081 | 3:66782644 |
| 15094 | 3:67125573 | 15179 | 3:69231454 | 15232 | 3:70770801 | 15263 | 3:72024952 | 15274 | 3:72399723 |
| 15277 | 3:72561296 | 15278 | 3:72658767 | 15282 | 3:72860663 | 15323 | 3:74163128 | 15334 | 3:74460656 |
| 15335 | 3:74560466 | 15373 | 3:75613962 | 15388 | 3:75959257 | 15396 | 3:76253283 | 15407 | 3:76546947 |
| 15518 | 3:79853923 | 15527 | 3:80073268 | 15551 | 3:80845482 | 15603 | 3:82127843 | 15647 | 3:83276598 |
| 15687 | 3:84374682 | 15749 | 3:85174617 | 15864 | 3:87868064 | 16131 | 3:93389797 | 16146 | 3:93816462 |
| 16173 | 3:94470905 | 16174 | 3:94570511 | 16205 | 3:95542077 | 16234 | 3:96375723 | 16236 | 3:96516771 |
| 16319 | 3:98506422 | 16487 | 3:101953130 | 16551 | 3:103287865 | 17051 | 3:112888751 | 17107 | 3:113766186 |
| 17462 | 3:119142510 | 17463 | 3:119241254 | 17465 | 3:119312183 | 17466 | 3:119337850 | 17471 | 3:119559889 |
| 17490 | 3:120011571 | 17505 | 3:120402369 | 17508 | 3:120529061 | 17527 | 4:196536 | 17528 | 4:280153 |
| 17533 | 4:535625 | 17539 | 4:791948 | 17549 | 4:1260383 | 17552 | 4:1417558 | 17600 | 4:2614737 |
| 17745 | 4:5912982 | 17995 | 4:11174189 | 18042 | 4:12473770 | 18087 | 4:13462303 | 18127 | 4:14704975 |
| 18131 | 4:14787436 | 18168 | 4:15690889 | 18178 | 4:16124536 | 18250 | 4:17859359 | 18322 | 4:19353388 |
| 18383 | 4:20445754 | 18433 | 4:21454127 | 18434 | 4:21541657 | 18445 | 4:21872378 | 18462 | 4:22281058 |
| 18501 | 4:23239526 | 18518 | 4:23775334 | 18529 | 4:24057792 | 18561 | 4:25063606 | 18581 | 4:25549415 |
| 18709 | 4:28892826 | 18713 | 4:29073099 | 18729 | 4:29642872 | 18740 | 4:30014475 | 18741 | 4:30101996 |
| 18846 | 4:32916549 | 18904 | 4:34496286 | 18926 | 4:35328524 | 19007 | 4:37643101 | 19054 | 4:38955171 |
| 19106 | 4:40318271 | 19156 | 4:41249802 | 19160 | 4:41529195 | 19161 | 4:41619769 | 19165 | 4:41769265 |
| 19169 | 4:41869434 | 19170 | 4:41950512 | 19184 | 4:42417863 | 19237 | 4:43892488 | 19281 | 4:45005168 |
| 19322 | 4:46242543 | 19325 | 4:46409911 | 19326 | 4:46490707 | 19407 | 4:48377180 | 19408 | 4:48467232 |
| 19487 | 4:50087185 | 19488 | 4:50186156 | 19499 | 4:50633232 | 19510 | 4:50996653 | 19537 | 4:51864991 |
| 19612 | 4:53951273 | 19613 | 4:54035561 | 19616 | 4:54191462 | 19618 | 4:54332820 | 19619 | 4:54391051 |
| 19629 | 4:54743084 | 19640 | 4:55160033 | 19642 | 4:55344926 | 19687 | 4:56506421 | 19766 | 4:59069599 |
| 19793 | 4:59746110 | 19805 | 4:60065778 | 19828 | 4:60746730 | 19861 | 4:61706369 | 19892 | 4:62405503 |
| 19897 | 4:62630427 | 19898 | 4:62713783 | 19987 | 4:65201660 | 19989 | 4:65245742 | 19990 | 4:65343359 |
| 19991 | 4:65436101 | 19993 | 4:65541264 | 20071 | 4:67301224 | 20088 | 4:67785324 | 20101 | 4:68075914 |
| 20135 | 4:69146493 | 20160 | 4:69711048 | 20161 | 4:69810925 | 20204 | 4:70819803 | 20231 | 4:71683652 |
| 20252 | 4:72269219 | 20253 | 4:72368269 | 20301 | 4:73885846 | 20309 | 4:74101412 | 20450 | 4:77371853 |
| 20475 | 4:77790836 | 20529 | 4:79467619 | 20647 | 4:82182401 | 20730 | 4:84549829 | 20733 | 4:84649340 |
| 20835 | 4:87133726 | 20859 | 4:87827889 | 20910 | 4:89303585 | 20944 | 4:90250683 | 20961 | 4:90701139 |
| 20978 | 4:91069757 | 21084 | 4:93203600 | 21136 | 4:94310305 | 21160 | 4:95074007 | 21254 | 4:96620212 |
| 21255 | 4:96718455 | 21485 | 4:99029738 | 21489 | 4:99310528 | 21491 | 4:99423168 | 21610 | 4:101894551 |
| 21616 | 4:102178027 | 21696 | 4:104152919 | 21762 | 4:105592184 | 21763 | 4:105606470 | 21764 | 4:105706248 |
| 21771 | 4:105878830 | 21772 | 4:105960151 | 21945 | 4:109512073 | 22167 | 4:113058361 | 22168 | 4:113156968 |
| 22546 | 4:119885612 | 22565 | 5:195803 | 22566 | 5:239346 | 22567 | 5:334945 | 22569 | 5:484209 |
| 22570 | 5:577221 | 22620 | 5:1547817 | 22706 | 5:3643809 | 22817 | 5:6128913 | 22818 | 5:6209013 |
| 23050 | 5:11616643 | 23133 | 5:13446994 | 23146 | 5:13786322 | 23170 | 5:14539492 | 23173 | 5:14638005 |
| 23183 | 5:14893917 | 23248 | 5:16007217 | 23258 | 5:16431067 | 23300 | 5:17571373 | 23303 | 5:17775869 |
| 23312 | 5:18094567 | 23332 | 5:18790466 | 23643 | 5:26034515 | 23644 | 5:26061374 | 23645 | 5:26151322 |
| 23657 | 5:26429773 | 23772 | 5:28790811 | 23781 | 5:29074134 | 23866 | 5:30986242 | 23867 | 5:31079915 |
| 23893 | 5:31837129 | 23983 | 5:34087072 | 23986 | 5:34286241 | 23987 | 5:34305687 | 23988 | 5:34375513 |
| 23989 | 5:34474025 | 23993 | 5:34687154 | 24020 | 5:35185968 | 24025 | 5:35351867 | 24034 | 5:35659101 |
| 24055 | 5:36320021 | 24070 | 5:36698940 | 24073 | 5:36843791 | 24100 | 5:37690831 | 24156 | 5:38824022 |
| 24222 | 5:40535193 | 24224 | 5:40667896 | 24269 | 5:42106898 | 24276 | 5:42475432 | 24298 | 5:42980132 |
| 24308 | 5:43193547 | 24330 | 5:43788410 | 24355 | 5:44185303 | 24356 | 5:44283317 | 24411 | 5:45870645 |
| 24496 | 5:47715438 | 24508 | 5:48068765 | 24588 | 5:50328496 | 24634 | 5:51301107 | 24681 | 5:52618260 |
| 24684 | 5:52742025 | 24687 | 5:52972061 | 24709 | 5:53722470 | 24719 | 5:53987712 | 24800 | 5:55749160 |
| 24856 | 5:57057069 | 24875 | 5:57516539 | 24885 | 5:57746077 | 24886 | 5:57832714 | 24887 | 5:57930196 |
| 24906 | 5:58383434 | 24909 | 5:58493473 | 24913 | 5:58628294 | 24914 | 5:58693980 | 24917 | 5:58889899 |
| 24918 | 5:58986587 | 24932 | 5:59477581 | 24935 | 5:59606022 | 24937 | 5:59692009 | 24938 | 5:59786270 |
| 24966 | 5:60382341 | 24967 | 5:60450259 | 25017 | 5:61851862 | 25115 | 5:64573229 | 25212 | 5:67004367 |
| 25214 | 5:67099597 | 25215 | 5:67183935 | 25245 | 5:67610309 | 25287 | 5:68696282 | 25352 | 5:70049824 |
| 25368 | 5:70612128 | 25480 | 5:72974385 | 25499 | 5:73530687 | 25534 | 5:74251346 | 25535 | 5:74343381 |
| 25593 | 5:75456468 | 25594 | 5:75473431 | 25595 | 5:75561363 | 25668 | 5:77473876 | 25669 | 5:77528763 |
| 25676 | 5:77903820 | 25690 | 5:78146870 | 25691 | 5:78242136 | 25730 | 5:79116954 | 25737 | 5:79370071 |
| 25738 | 5:79464943 | 25739 | 5:79516884 | 25762 | 5:80354597 | 25763 | 5:80452900 | 25786 | 5:81078405 |
| 25790 | 5:81259940 | 25866 | 5:82675565 | 25872 | 5:82889086 | 26084 | 5:84839297 | 26119 | 5:85681634 |
| 26136 | 5:86133720 | 26140 | 5:86239067 | 26194 | 5:87412820 | 26322 | 5:89485893 | 26392 | 5:91260025 |
| 26475 | 5:92686818 | 26708 | 5:97550215 | 26787 | 5:99116271 | 26788 | 5:99137116 | 26789 | 5:99143867 |
| 26790 | 5:99243441 | 26791 | 5:99314005 | 26931 | 5:102029541 | 26933 | 5:102150740 | 26938 | 5:102366923 |
| 26949 | 5:102638331 | 26950 | 5:102641378 | 26951 | 5:102662950 | 26952 | 5:102812214 | 26953 | 5:102878588 |
| 26954 | 5:102970619 | 26955 | 5:103010571 | 27120 | 5:106009983 | 27279 | 5:108384166 | 27298 | 5:108674540 |
| 27332 | 5:109217271 | 27333 | 5:109314750 | 27544 | 5:113514180 | 27545 | 5:113554598 | 27575 | 5:114440055 |
| 27576 | 5:114536143 | 27578 | 5:114640058 | 27595 | 5:115151924 | 27738 | 5:116870418 | 27938 | 6:97487 |
| 27946 | 6:394469 | 27947 | 6:492748 | 27979 | 6:1193084 | 28037 | 6:2239422 | 28060 | 6:2768563 |
| 28130 | 6:3837662 | 28184 | 6:5212458 | 28195 | 6:5517626 | 28196 | 6:5615037 | 28314 | 6:8143447 |
| 28328 | 6:8615351 | 28329 | 6:8684262 | 28437 | 6:11262662 | 28551 | 6:13520510 | 28553 | 6:13697509 |
| 28571 | 6:14245516 | 28744 | 6:17861677 | 28918 | 6:21562233 | 29086 | 6:25221507 | 29099 | 6:25661883 |
| 29133 | 6:26462640 | 29181 | 6:27717652 | 29384 | 6:31231051 | 29419 | 6:32134214 | 29424 | 6:32438836 |
| 29434 | 6:32698791 | 29437 | 6:32783759 | 29438 | 6:32866982 | 32354 | 6:44532925 | 32523 | 6:45804379 |
| 32559 | 6:46543851 | 32571 | 6:46849949 | 32625 | 6:48139435 | 32639 | 6:48619247 | 32722 | 6:50593610 |
| 32763 | 6:51940574 | 32772 | 6:52186933 | 32883 | 6:54324568 | 32961 | 6:55908779 | 32979 | 6:56522044 |
| 33028 | 6:57631443 | 33029 | 6:57728837 | 33054 | 6:58492711 | 33067 | 6:58822636 | 33096 | 6:59668604 |
| 33161 | 6:61063375 | 33203 | 6:62168026 | 33214 | 6:62509742 | 33219 | 6:62716293 | 33266 | 6:64072884 |
| 33270 | 6:64250794 | 33339 | 6:66605824 | 33359 | 6:67153345 | 33441 | 6:69518831 | 33443 | 6:69655680 |
| 33453 | 6:69965893 | 33454 | 6:70058818 | 33456 | 6:70166659 | 33473 | 6:70643646 | 33511 | 6:71802143 |
| 33591 | 6:74410889 | 33596 | 6:74629845 | 33599 | 6:74741424 | 33617 | 6:75350029 | 33619 | 6:75446552 |
| 33621 | 6:75621675 | 33636 | 6:75990894 | 33639 | 6:76209600 | 33640 | 6:76275919 | 33643 | 6:76450277 |
| 33706 | 6:78238972 | 33708 | 6:78363832 | 33713 | 6:78527838 | 33724 | 6:79038243 | 33745 | 6:79831508 |
| 33795 | 6:81332996 | 33807 | 6:81870711 | 33808 | 6:81964290 | 33862 | 6:83422593 | 33883 | 6:83749295 |
| 33906 | 6:84274386 | 33923 | 6:84775788 | 33989 | 6:86113898 | 34365 | 6:93730394 | 34406 | 6:94508641 |
| 34407 | 6:94604426 | 34408 | 6:94671907 | 34418 | 6:95161943 | 34422 | 6:95280868 | 34621 | 6:99278886 |
| 34645 | 6:100084140 | 34895 | 6:104783662 | 34926 | 6:105376492 | 35015 | 6:107674685 | 35016 | 6:107769675 |
| 35048 | 6:108791274 | 35098 | 6:109783321 | 35256 | 6:113049419 | 35257 | 6:113144284 | 35385 | 6:115461946 |
| 35450 | 6:116698963 | 35451 | 6:116752334 | 35452 | 6:116848436 | 35454 | 6:117023687 | 35455 | 6:117121869 |
| 35460 | 6:117289738 | 35461 | 6:117373173 | 35468 | 6:117512770 | 35471 | 6:117661215 | 35472 | 6:117698401 |
| 35473 | 6:117792640 | 35474 | 7:80741 | 35475 | 7:164546 | 35720 | 7:4691508 | 35922 | 7:7350133 |
| 35945 | 7:8005827 | 35954 | 7:8250300 | 35957 | 7:8423976 | 35962 | 7:8644832 | 35963 | 7:8715092 |
| 35964 | 7:8798532 | 35965 | 7:8829774 | 35967 | 7:9006597 | 35968 | 7:9034045 | 35969 | 7:9111685 |
| 35970 | 7:9205519 | 35971 | 7:9294350 | 35972 | 7:9323285 | 35973 | 7:9420386 | 35974 | 7:9427375 |
| 35975 | 7:9522686 | 35976 | 7:9608447 | 35980 | 7:9782711 | 35981 | 7:9853483 | 35982 | 7:9893322 |
| 35983 | 7:9935560 | 35987 | 7:10196676 | 35988 | 7:10292183 | 35993 | 7:10432630 | 35994 | 7:10494698 |
| 35995 | 7:10588854 | 36000 | 7:10694692 | 36001 | 7:10709248 | 36002 | 7:10799397 | 36003 | 7:10898948 |
| 36004 | 7:10972220 | 36034 | 7:11621411 | 36038 | 7:11733951 | 36290 | 7:15957756 | 36351 | 7:17374656 |
| 36373 | 7:17800196 | 36382 | 7:18107639 | 36530 | 7:20567856 | 36589 | 7:21886187 | 36612 | 7:22364050 |
| 36643 | 7:23141409 | 36693 | 7:24266298 | 36997 | 7:31102265 | 37168 | 7:34931021 | 37197 | 7:35685794 |
| 37213 | 7:36102653 | 37218 | 7:36242365 | 37331 | 7:38727405 | 37345 | 7:39057017 | 37432 | 7:40812159 |
| 37449 | 7:41102749 | 37450 | 7:41174068 | 37456 | 7:41391176 | 37460 | 7:41530820 | 37465 | 7:41621787 |
| 37485 | 7:42209152 | 37489 | 7:42394156 | 37545 | 7:43685836 | 37553 | 7:43941722 | 37559 | 7:44261005 |
| 37564 | 7:44505025 | 37616 | 7:45789210 | 37618 | 7:45899026 | 37622 | 7:46066923 | 37702 | 7:47884765 |
| 37704 | 7:47992422 | 37706 | 7:48152895 | 37757 | 7:49264671 | 37763 | 7:49508927 | 37764 | 7:49551981 |
| 37765 | 7:49632647 | 37766 | 7:49732360 | 37767 | 7:49825415 | 37772 | 7:50083648 | 37773 | 7:50141438 |
| 37774 | 7:50238296 | 37775 | 7:50295962 | 37776 | 7:50392227 | 37778 | 7:50523822 | 37782 | 7:50734140 |
| 37785 | 7:50857210 | 37786 | 7:50956025 | 37795 | 7:51298758 | 37805 | 7:51627529 | 37808 | 7:51770556 |
| 37810 | 7:51904626 | 37877 | 7:52516644 | 37913 | 7:53384544 | 37926 | 7:53682528 | 37936 | 7:54073527 |
| 38031 | 7:56472724 | 38061 | 7:57477755 | 38086 | 7:58105186 | 38087 | 7:58204392 | 38167 | 7:59670910 |
| 38195 | 7:60290921 | 38291 | 7:62241248 | 38363 | 7:63608853 | 38381 | 7:64051258 | 38502 | 7:66283774 |
| 38508 | 7:66535109 | 38570 | 7:68229559 | 38620 | 7:69315564 | 38662 | 7:70240253 | 38678 | 7:70856473 |
| 38773 | 7:73340932 | 38774 | 7:73425725 | 38792 | 7:73921199 | 38812 | 7:74461479 | 39002 | 7:78855864 |
| 39182 | 7:82082096 | 39313 | 7:85461499 | 39332 | 7:86158728 | 39339 | 7:86335874 | 39344 | 7:86445479 |
| 39366 | 7:86982142 | 39476 | 7:89945029 | 39477 | 7:90039805 | 39669 | 7:93367867 | 39716 | 7:94784594 |
| 40029 | 7:98642157 | 40031 | 7:98712459 | 40032 | 7:98793113 | 40136 | 7:101498466 | 40233 | 7:103750242 |
| 40234 | 7:103835986 | 40277 | 7:105099277 | 40483 | 7:110118836 | 40484 | 7:110198554 | 40485 | 7:110298545 |
| 40486 | 7:110364995 | 40487 | 7:110462011 | 40488 | 7:110524775 | 40489 | 7:110616075 | 40491 | 8:119077 |
| 40492 | 8:204055 | 40502 | 8:510254 | 40549 | 8:1447144 | 40550 | 8:1539600 | 40593 | 8:2298980 |
| 40618 | 8:3109616 | 40678 | 8:4419528 | 40679 | 8:4488605 | 40713 | 8:5356367 | 40792 | 8:7353398 |
| 40816 | 8:7715961 | 40887 | 8:9573171 | 40913 | 8:10172186 | 41018 | 8:12543611 | 41019 | 8:12642666 |
| 41081 | 8:14746899 | 41091 | 8:15001201 | 41101 | 8:15260009 | 41198 | 8:17406666 | 41267 | 8:19472727 |
| 41298 | 8:20253347 | 41380 | 8:22549276 | 41383 | 8:22747845 | 41388 | 8:22913643 | 41395 | 8:23313119 |
| 41446 | 8:24571671 | 41627 | 8:27179633 | 41661 | 8:28342382 | 41677 | 8:28819278 | 41709 | 8:29645861 |
| 41763 | 8:30913973 | 41814 | 8:32226362 | 41825 | 8:32643213 | 41830 | 8:32879092 | 41831 | 8:32974702 |
| 41833 | 8:33127475 | 41844 | 8:33407993 | 41845 | 8:33470771 | 41900 | 8:34950717 | 41905 | 8:35118252 |
| 41930 | 8:35658609 | 42020 | 8:37747312 | 42042 | 8:38560565 | 42053 | 8:38837520 | 42054 | 8:38936996 |
| 42058 | 8:39131324 | 42203 | 8:42579916 | 42214 | 8:42818923 | 42264 | 8:44335672 | 42287 | 8:45011011 |
| 42294 | 8:45233429 | 42414 | 8:47856381 | 42435 | 8:48611381 | 42541 | 8:51528439 | 42598 | 8:52866048 |
| 42705 | 8:55793930 | 42720 | 8:56175135 | 42793 | 8:57947632 | 42804 | 8:58378680 | 42806 | 8:58511840 |
| 42900 | 8:60710884 | 42937 | 8:61860162 | 42990 | 8:63095889 | 42996 | 8:63293941 | 42997 | 8:63391647 |
| 43046 | 8:64688332 | 43070 | 8:65351408 | 43083 | 8:65593070 | 43086 | 8:65766665 | 43108 | 8:66276105 |
| 43137 | 8:66960690 | 43199 | 8:68392696 | 43246 | 8:69535824 | 43279 | 8:70389972 | 43369 | 8:72702268 |
| 43466 | 8:75072947 | 43494 | 8:76094638 | 43555 | 8:77086614 | 43558 | 8:77243628 | 43569 | 8:77734663 |
| 43571 | 8:77844702 | 43601 | 8:78788179 | 43610 | 8:79140878 | 43615 | 8:79417890 | 43732 | 8:82254632 |
| 43780 | 8:83455155 | 43784 | 8:83630961 | 43864 | 8:85507118 | 43904 | 8:86456661 | 43907 | 8:86622904 |
| 43962 | 8:87959678 | 43976 | 8:88341264 | 44029 | 8:89430089 | 44104 | 8:91188437 | 44105 | 8:91284358 |
| 44119 | 8:91608918 | 44148 | 8:92091641 | 44201 | 8:93754674 | 44213 | 8:94306363 | 44214 | 8:94402192 |
| 44215 | 8:94469316 | 44280 | 8:96346983 | 44433 | 8:99864196 | 44481 | 8:101198784 | 44501 | 8:101698974 |
| 44683 | 8:104921232 | 44740 | 8:106178029 | 44742 | 8:106321176 | 44817 | 8:108369912 | 44818 | 8:108466305 |
| 44826 | 8:108830921 | 44827 | 8:108930028 | 44840 | 8:109399618 | 44844 | 8:109663372 | 44876 | 8:110607864 |
| 44902 | 8:111508670 | 44903 | 8:111524854 | 44904 | 8:111672008 | 44905 | 8:111767142 | 44906 | 8:111818202 |
| 44907 | 8:111891683 | 44908 | 8:111919894 | 44909 | 8:112058818 | 44910 | 8:112150362 | 44911 | 8:112232760 |
| 44912 | 8:112294473 | 44913 | 8:112461632 | 44914 | 8:112537285 | 44915 | 8:112604404 | 44916 | 8:112658175 |
| 44917 | 8:112746559 | 44918 | 8:112818233 | 44919 | 8:112865068 | 44920 | 8:112932415 | 44921 | 8:112969679 |
| 44922 | 8:112981176 | 44923 | 8:113097953 | 44924 | 8:113163827 | 44925 | 8:113180320 | 44926 | 8:113252524 |
| 44930 | 9:190038 | 44931 | 9:289864 | 45031 | 9:2539725 | 45074 | 9:3743248 | 45089 | 9:4213848 |
| 45112 | 9:4988015 | 45135 | 9:5683174 | 45179 | 9:7188401 | 45252 | 9:8906729 | 45274 | 9:9555271 |
| 45378 | 9:10911653 | 45502 | 9:13481091 | 45518 | 9:13965555 | 45582 | 9:15291327 | 45748 | 9:19358233 |
| 45798 | 9:20933619 | 45807 | 9:21235916 | 45808 | 9:21329366 | 46023 | 9:26340073 | 46029 | 9:26560747 |
| 46194 | 9:29938464 | 46280 | 9:31821673 | 46371 | 9:34153811 | 46384 | 9:34492853 | 46503 | 9:37375637 |
| 46515 | 9:37681606 | 46604 | 9:39416078 | 46605 | 9:39496518 | 46691 | 9:41269453 | 46692 | 9:41353434 |
| 46707 | 9:41741270 | 46708 | 9:41834289 | 46710 | 9:41949048 | 46773 | 9:43557020 | 46780 | 9:43780420 |
| 46834 | 9:45079509 | 46835 | 9:45168173 | 46839 | 9:45353183 | 46856 | 9:45976363 | 46867 | 9:46356308 |
| 46879 | 9:46772812 | 46892 | 9:47186026 | 46935 | 9:48538260 | 47033 | 9:51244082 | 47100 | 9:53108085 |
| 47125 | 9:53577095 | 47177 | 9:55074875 | 47178 | 9:55120285 | 47179 | 9:55219943 | 47205 | 9:56052958 |
| 47226 | 9:56606356 | 47254 | 9:57407794 | 47256 | 9:57496462 | 47281 | 9:58031623 | 47311 | 9:59018652 |
| 47408 | 9:61120108 | 47411 | 9:61356232 | 47442 | 9:62281541 | 47443 | 9:62376661 | 47493 | 9:63781683 |
| 47496 | 9:63979099 | 47499 | 9:64121913 | 47535 | 9:64946776 | 47643 | 9:66726405 | 47644 | 9:66806876 |
| 47646 | 9:66932759 | 47655 | 9:67186941 | 47715 | 9:68688996 | 47850 | 9:71852129 | 47857 | 9:72065985 |
| 47858 | 9:72161733 | 47894 | 9:73058318 | 47968 | 9:74896771 | 48054 | 9:77165874 | 48179 | 9:79135168 |
| 48188 | 9:79446217 | 48426 | 9:84561403 | 48431 | 9:84647624 | 48432 | 9:84745238 | 48521 | 9:86545282 |
| 48523 | 9:86670733 | 48544 | 9:87047352 | 48545 | 9:87102465 | 48546 | 9:87196437 | 48547 | 9:87295910 |
| 48615 | 9:88318893 | 48799 | 9:91914972 | 48879 | 9:93747256 | 49154 | 9:98379141 | 49230 | 9:99867971 |
| 49483 | 9:104102942 | 49484 | 9:104107451 | 49486 | 9:104142058 | 49488 | 9:104277105 | 49495 | 9:104453514 |
| 49497 | 9:104550915 | 49498 | 9:104641226 | 49499 | 9:104641308 | 49500 | 9:104641495 | 49502 | 10:184460 |
| 49503 | 10:213217 | 49504 | 10:295234 | 49506 | 10:344867 | 49507 | 10:435671 | 49566 | 10:1764102 |
| 49836 | 10:6862089 | 49850 | 10:7338516 | 49919 | 10:8633030 | 49991 | 10:10372137 | 50045 | 10:11267448 |
| 50115 | 10:12952255 | 50434 | 10:18833922 | 50435 | 10:18841050 | 50436 | 10:18845495 | 50437 | 10:18848616 |
| 50438 | 10:18854031 | 50469 | 10:19429779 | 50470 | 10:19516186 | 50638 | 10:22676353 | 50639 | 10:22775635 |
| 50642 | 10:22836164 | 50643 | 10:22909428 | 50646 | 10:23059842 | 50647 | 10:23066056 | 50648 | 10:23228975 |
| 50649 | 10:23268766 | 50650 | 10:23368614 | 50652 | 10:23464004 | 50653 | 10:23533419 | 50654 | 10:23627755 |
| 50655 | 10:23671760 | 50656 | 10:23769788 | 50657 | 10:23771464 | 50658 | 10:24790738 | 50659 | 10:24799735 |
| 50660 | 10:24818929 | 50661 | 10:24913820 | 50662 | 10:24923736 | 50663 | 10:25022602 | 50664 | 10:25057985 |
| 50665 | 10:25156372 | 50666 | 10:25164147 | 50667 | 10:25241806 | 50668 | 10:25341513 | 50680 | 10:25758684 |
| 50778 | 10:27556986 | 50900 | 10:29890120 | 50926 | 10:30807403 | 51080 | 10:34810626 | 51145 | 10:36132543 |
| 51146 | 10:36213908 | 51160 | 10:36596113 | 51163 | 10:36787409 | 51240 | 10:38618727 | 51259 | 10:39073733 |
| 51260 | 10:39169136 | 51284 | 10:39715696 | 51292 | 10:39993149 | 51302 | 10:40306626 | 51390 | 10:42652463 |
| 51403 | 10:43115839 | 51417 | 10:43643966 | 51433 | 10:44096569 | 51474 | 10:45124642 | 51490 | 10:45511746 |
| 51745 | 10:50796271 | 51861 | 10:53660012 | 51942 | 10:56043883 | 51992 | 10:57210937 | 52035 | 10:58319989 |
| 52040 | 10:58481438 | 52054 | 10:58874663 | 52071 | 10:59336872 | 52092 | 10:60028136 | 52123 | 10:61231374 |
| 52126 | 10:61443371 | 52158 | 10:62042620 | 52193 | 10:62708109 | 52228 | 10:63495064 | 52236 | 10:63804854 |
| 52247 | 10:64189747 | 52294 | 10:65251624 | 52312 | 10:65561116 | 52344 | 10:66259714 | 52392 | 10:67327408 |
| 52523 | 10:70522773 | 52524 | 10:70601203 | 52526 | 10:70690873 | 52527 | 10:70781098 | 52548 | 10:71377715 |
| 52590 | 10:72631813 | 52591 | 10:72727860 | 52596 | 10:72983865 | 52597 | 10:73076966 | 52643 | 10:74135935 |
| 52664 | 10:74941316 | 52700 | 10:75976569 | 52736 | 10:76707628 | 53015 | 10:82572013 | 53034 | 10:83144860 |
| 53169 | 10:86010466 | 53423 | 10:91110721 | 53454 | 10:92010714 | 53519 | 10:93279984 | 53520 | 10:93370297 |
| 53640 | 10:96340328 | 53705 | 10:97729030 | 53779 | 10:99321961 | 53802 | 10:99841614 | 53981 | 10:103277404 |
| 54006 | 11:514378 | 54266 | 11:4322857 | 54313 | 11:5395664 | 54364 | 11:6596735 | 54435 | 11:8259962 |
| 54438 | 11:8462700 | 54446 | 11:8761089 | 54705 | 11:14474472 | 54822 | 11:17458053 | 54838 | 11:17908654 |
| 54839 | 11:17983413 | 54843 | 11:18162535 | 54847 | 11:18333639 | 54889 | 11:19456930 | 54900 | 11:19732194 |
| 54920 | 11:20390071 | 54993 | 11:22091427 | 55041 | 11:23355951 | 55079 | 11:24188498 | 55126 | 11:25171864 |
| 55148 | 11:25792655 | 55180 | 11:26465214 | 55181 | 11:26466574 | 55182 | 11:26531940 | 55361 | 11:30575172 |
| 55422 | 11:32139468 | 55488 | 11:33907492 | 55506 | 11:34748337 | 55580 | 11:36801995 | 55638 | 11:37982330 |
| 55648 | 11:38441743 | 55686 | 11:39673530 | 55712 | 11:40436923 | 55715 | 11:40647883 | 55794 | 11:42338919 |
| 55853 | 11:43645607 | 55866 | 11:44133002 | 55918 | 11:45401293 | 55920 | 11:45518720 | 55922 | 11:45703344 |
| 55923 | 11:45779776 | 55956 | 11:46344663 | 56060 | 11:48391618 | 56182 | 11:50484454 | 56211 | 11:51437117 |
| 56233 | 11:52229998 | 56236 | 11:52492485 | 56254 | 11:53121751 | 56260 | 11:53366816 | 56270 | 11:53745957 |
| 56342 | 11:55279305 | 56373 | 11:55996334 | 56379 | 11:56241704 | 56381 | 11:56406566 | 56386 | 11:56550578 |
| 56393 | 11:56780643 | 56413 | 11:57282162 | 56423 | 11:57597675 | 56424 | 11:57696611 | 56449 | 11:58487002 |
| 56502 | 11:59891961 | 56507 | 11:60131924 | 56508 | 11:60226569 | 56583 | 11:61202324 | 56584 | 11:61289988 |
| 56586 | 11:61410046 | 56587 | 11:61495664 | 56592 | 11:61644339 | 56593 | 11:61726332 | 56717 | 11:64661124 |
| 56765 | 11:65925707 | 56800 | 11:66728012 | 56888 | 11:68240104 | 56900 | 11:68629264 | 56946 | 11:69472443 |
| 57044 | 11:70953054 | 57115 | 11:72333655 | 57123 | 11:72679475 | 57165 | 11:73412263 | 57169 | 11:73601533 |
| 57220 | 11:74558447 | 57221 | 11:74602747 | 57223 | 11:74706334 | 57268 | 11:75776010 | 57283 | 11:76197120 |
| 57467 | 11:79506772 | 57675 | 11:84372612 | 57684 | 11:84807135 | 57765 | 11:86512168 | 57810 | 11:87538574 |
| 57811 | 11:87624731 | 57982 | 11:90885707 | 58110 | 11:93370799 | 58146 | 11:94432248 | 58203 | 11:95957372 |
| 58367 | 11:99599606 | 58410 | 11:100441246 | 58478 | 11:101655272 | 58566 | 11:103219634 | 58606 | 11:104009437 |
| 58677 | 11:105366441 | 58680 | 11:105457027 | 58708 | 11:105856659 | 58709 | 11:105955321 | 58714 | 11:106183172 |
| 58716 | 11:106290122 | 58718 | 11:106431008 | 58721 | 11:106566471 | 58728 | 11:106719246 | 58730 | 11:106872762 |
| 58732 | 12:91462 | 58765 | 12:1113931 | 58768 | 12:1245380 | 58884 | 12:3705269 | 58963 | 12:5912555 |
| 58975 | 12:6345821 | 58982 | 12:6709902 | 58998 | 12:7126322 | 58999 | 12:7224038 | 59220 | 12:12834462 |
| 59327 | 12:15203798 | 59530 | 12:19527022 | 59607 | 12:21552423 | 59610 | 12:21691586 | 59611 | 12:21787162 |
| 59641 | 12:22535408 | 59688 | 12:23635155 | 59689 | 12:23728488 | 59791 | 12:25996134 | 59792 | 12:26092741 |
| 59794 | 12:26216578 | 59804 | 12:26581859 | 59875 | 12:28358077 | 59905 | 12:29299176 | 59926 | 12:29941712 |
| 59950 | 12:30491646 | 60005 | 12:31739889 | 60020 | 12:32049680 | 60093 | 12:33950251 | 60098 | 12:34160176 |
| 60110 | 12:34560071 | 60129 | 12:35057478 | 60167 | 12:35870799 | 60171 | 12:36164862 | 60174 | 12:36313336 |
| 60188 | 12:36646069 | 60194 | 12:36915655 | 60217 | 12:37496437 | 60218 | 12:37593149 | 60219 | 12:37662796 |
| 60221 | 12:37752580 | 60223 | 12:37929010 | 60224 | 12:37976297 | 60225 | 12:38053259 | 60230 | 12:38238813 |
| 60242 | 12:38895814 | 60247 | 12:39058185 | 60250 | 12:39172935 | 60271 | 12:39830361 | 60272 | 12:39927124 |
| 60297 | 12:40565431 | 60302 | 12:40827682 | 60314 | 12:41265419 | 60318 | 12:41435539 | 60322 | 12:41606679 |
| 60337 | 12:42104689 | 60343 | 12:42399827 | 60348 | 12:42626305 | 60349 | 12:42724793 | 60353 | 12:42919011 |
| 60357 | 12:43020517 | 60359 | 12:43190819 | 60369 | 12:43612489 | 60370 | 12:43698277 | 60374 | 12:43993681 |
| 60398 | 12:44816702 | 60406 | 12:45079833 | 60408 | 12:45170993 | 60432 | 12:45962853 | 60433 | 12:46045130 |
| 60515 | 12:48438301 | 60537 | 12:48906417 | 60622 | 12:50638774 | 60623 | 12:50712724 | 60795 | 12:54440430 |
| 60879 | 12:56915140 | 60896 | 12:57520936 | 60897 | 12:57618510 | 60917 | 12:58198920 | 60927 | 12:58558724 |
| 60980 | 12:60328402 | 60984 | 12:60431728 | 60991 | 12:60833351 | 60998 | 12:61166007 | 61017 | 12:61818463 |
| 61069 | 12:63224791 | 61091 | 12:63866391 | 61114 | 12:64636179 | 61130 | 12:65016774 | 61202 | 12:66945480 |
| 61203 | 12:67032666 | 61345 | 12:69830668 | 61346 | 12:70008764 | 61347 | 12:70092121 | 61349 | 12:70199557 |
| 61350 | 12:70296346 | 61351 | 12:70314066 | 61352 | 12:70373860 | 61354 | 12:70528567 | 61355 | 12:70625414 |
| 61356 | 12:70723529 | 61359 | 12:70754288 | 61360 | 12:70832160 | 61361 | 12:70921442 | 61362 | 12:71020218 |
| 61363 | 12:71027334 | 61364 | 12:71106543 | 61365 | 12:71199759 | 61375 | 12:71408795 | 61376 | 12:71457224 |
| 61377 | 12:71551210 | 61384 | 12:71729441 | 61392 | 12:71965489 | 61393 | 12:72058978 | 61394 | 12:72147420 |
| 61395 | 12:72230980 | 61396 | 12:72265152 | 61397 | 12:72337871 | 61398 | 12:72374872 | 61399 | 12:72490055 |
| 61400 | 12:72498314 | 61401 | 12:72637442 | 61402 | 12:72712776 | 61403 | 12:72812633 | 61902 | 12:83578230 |
| 62097 | 12:87141742 | 62098 | 13:299895 | 62105 | 13:682778 | 62106 | 13:745802 | 62125 | 13:1154063 |
| 62224 | 13:2881031 | 62225 | 13:2975142 | 62280 | 13:4395240 | 62283 | 13:4531236 | 62431 | 13:8202540 |
| 62466 | 13:9078686 | 62545 | 13:10762211 | 62546 | 13:10778845 | 62548 | 13:10867741 | 62554 | 13:11145952 |
| 62555 | 13:11222783 | 62556 | 13:11320371 | 62557 | 13:11410445 | 62570 | 13:12019870 | 62634 | 13:13812138 |
| 62650 | 13:14311743 | 62692 | 13:15465789 | 62712 | 13:16020130 | 62799 | 13:17568011 | 62801 | 13:17588165 |
| 62835 | 13:18353118 | 63019 | 13:21321597 | 63087 | 13:23067175 | 63093 | 13:23318050 | 63105 | 13:23791918 |
| 63109 | 13:23900966 | 63124 | 13:24343896 | 63189 | 13:25699872 | 63323 | 13:29049594 | 63392 | 13:30294381 |
| 63393 | 13:30377425 | 63476 | 13:31944092 | 63477 | 13:32039794 | 63555 | 13:33385230 | 63576 | 13:34100138 |
| 63577 | 13:34193250 | 63578 | 13:34248535 | 63581 | 13:34427463 | 63706 | 13:37210289 | 63805 | 13:39259881 |
| 63838 | 13:39814181 | 64020 | 13:43484075 | 64302 | 13:48427057 | 64315 | 13:49071500 | 64316 | 13:49168510 |
| 64345 | 13:49747887 | 64346 | 13:49844262 | 64355 | 13:50115519 | 64357 | 13:50180207 | 64358 | 13:50268295 |
| 64372 | 13:50739564 | 64374 | 13:50832795 | 64381 | 13:51125057 | 64401 | 13:51523719 | 64402 | 13:51622569 |
| 64500 | 13:53510732 | 64501 | 13:53607473 | 64507 | 13:53822879 | 64519 | 13:54442428 | 64570 | 13:55504923 |
| 64652 | 13:57623027 | 64676 | 13:58277988 | 64741 | 13:59446084 | 64816 | 13:61419709 | 64828 | 13:61686658 |
| 64836 | 13:61962725 | 64928 | 13:63467047 | 64929 | 13:63559780 | 64949 | 13:64081941 | 65039 | 13:65929411 |
| 65137 | 13:68477424 | 65180 | 13:69587905 | 65181 | 13:69666536 | 65541 | 13:77055890 | 65542 | 13:77153724 |
| 65879 | 14:93414 | 65895 | 14:609870 | 65904 | 14:947222 | 66104 | 14:4750888 | 66610 | 14:13439017 |
| 66611 | 14:13473101 | 66612 | 14:13553001 | 66613 | 14:13632606 | 66614 | 14:13721229 | 66615 | 14:13734707 |
| 66616 | 14:13832328 | 66786 | 14:17490496 | 66822 | 14:18129262 | 66823 | 14:18227619 | 66827 | 14:18277849 |
| 66828 | 14:18376690 | 66874 | 14:19576484 | 67559 | 14:25819002 | 67636 | 14:26880974 | 67665 | 14:27799331 |
| 67800 | 14:30971186 | 67869 | 14:32582584 | 67898 | 14:33242218 | 67971 | 14:34837449 | 67972 | 14:34885142 |
| 67977 | 14:35171625 | 68004 | 14:35658219 | 68094 | 14:38119848 | 68123 | 14:38980550 | 68157 | 14:40059476 |
| 68173 | 14:40339877 | 68272 | 14:43115419 | 68466 | 14:47686908 | 68471 | 14:47920443 | 68533 | 14:49442868 |
| 68573 | 14:50805531 | 68645 | 14:52567142 | 68650 | 14:52653514 | 68657 | 14:52955563 | 68658 | 14:53054564 |
| 68676 | 14:53669390 | 68747 | 14:54918170 | 68781 | 14:55712044 | 68782 | 14:55807243 | 68783 | 14:55875273 |
| 68784 | 14:55953452 | 68824 | 14:57218378 | 68935 | 14:59606995 | 68952 | 14:60129379 | 68980 | 14:60666003 |
| 68981 | 14:60764297 | 69127 | 14:63797690 | 69169 | 14:64906621 | 69173 | 14:65094231 | 69187 | 14:65545962 |
| 69327 | 14:68518147 | 69328 | 14:68615752 | 69461 | 14:72182566 | 69496 | 14:72949159 | 69497 | 14:72993679 |
| 69514 | 14:73377613 | 69551 | 14:74402579 | 69585 | 14:75284663 | 69654 | 14:76712535 | 69927 | 15:278408 |
| 69928 | 15:364919 | 69935 | 15:581520 | 69938 | 15:790710 | 69940 | 15:921595 | 70011 | 15:2907387 |
| 70072 | 15:4118742 | 70298 | 15:5868982 | 70344 | 15:7132196 | 70468 | 15:9855083 | 70479 | 15:10238469 |
| 70485 | 15:10450890 | 70487 | 15:10557012 | 70500 | 15:11034094 | 70513 | 15:11544670 | 70515 | 15:11646233 |
| 70535 | 15:12337840 | 70536 | 15:12426564 | 70558 | 15:13317083 | 70564 | 15:13540062 | 70607 | 15:14553709 |
| 70611 | 15:14685598 | 70652 | 15:15637037 | 70768 | 15:17775660 | 70799 | 15:18549454 | 70821 | 15:19204706 |
| 70830 | 15:19463500 | 71142 | 15:25686598 | 71348 | 15:29961188 | 71383 | 15:30812283 | 71652 | 15:36545261 |
| 71720 | 15:37976104 | 71736 | 15:38441326 | 71771 | 15:39152334 | 71772 | 15:39251802 | 71782 | 15:39467016 |
| 72064 | 15:44893395 | 72102 | 15:45703046 | 72116 | 15:46016293 | 72117 | 15:46052010 | 72118 | 15:46128678 |
| 72120 | 15:46190128 | 72193 | 15:47315722 | 72195 | 15:47377732 | 72196 | 15:47473423 | 72199 | 15:47598007 |
| 72204 | 15:47732907 | 72264 | 15:48782827 | 72293 | 15:49903002 | 72297 | 15:50131403 | 72298 | 15:50228681 |
| 72321 | 15:50928742 | 72446 | 15:53225186 | 72482 | 15:53576274 | 72512 | 15:54128239 | 72554 | 15:55076548 |
| 72599 | 15:56354181 | 72600 | 15:56425647 | 72782 | 15:59421385 | 72830 | 15:60713798 | 72933 | 15:62959638 |
| 73204 | 15:68655064 | 73251 | 15:70097809 | 73252 | 15:70187975 | 73651 | 15:78826872 | 73652 | 15:78926080 |
| 73661 | 15:79238876 | 73664 | 15:79380785 | 73669 | 15:79544197 | 73670 | 15:79611716 | 73785 | 15:82130501 |
| 73795 | 15:82404865 | 73796 | 15:82425557 | 73797 | 15:82522759 | 73884 | 15:84032423 | 73887 | 15:84244168 |
| 73888 | 15:84260381 | 73889 | 15:84356903 | 73890 | 15:84447429 | 73891 | 15:84540077 | 73894 | 15:84686045 |
| 73895 | 15:84729195 | 73896 | 15:84804908 | 73897 | 15:84896615 | 73898 | 15:84939269 | 73901 | 16:338529 |
| 74061 | 16:3596934 | 74075 | 16:3906912 | 74153 | 16:5495502 | 74156 | 16:5663689 | 74159 | 16:5838621 |
| 74167 | 16:6306111 | 74197 | 16:6971134 | 74198 | 16:7015739 | 74199 | 16:7111115 | 74201 | 16:7234019 |
| 74221 | 16:7834741 | 74224 | 16:7996266 | 74228 | 16:8149785 | 74229 | 16:8194291 | 74256 | 16:9122788 |
| 74257 | 16:9217866 | 74265 | 16:9575503 | 74293 | 16:10558307 | 74409 | 16:13263254 | 74425 | 16:13790388 |
| 74496 | 16:15729876 | 74548 | 16:17488453 | 74550 | 16:17617156 | 74566 | 16:18152045 | 74575 | 16:18477012 |
| 74632 | 16:19928511 | 74709 | 16:21880848 | 74750 | 16:22924908 | 74816 | 16:24474002 | 74979 | 16:27452942 |
| 75007 | 16:28071735 | 75037 | 16:29095796 | 75043 | 16:29327042 | 75168 | 16:32723586 | 75199 | 16:33327718 |
| 75372 | 16:37660699 | 75428 | 16:38784562 | 75449 | 16:39690828 | 75490 | 16:40983811 | 75550 | 16:42163425 |
| 75555 | 16:42310324 | 75556 | 16:42386477 | 75560 | 16:42697916 | 75568 | 16:42993444 | 75580 | 16:43487631 |
| 75582 | 16:43668103 | 75584 | 16:43759140 | 75585 | 16:43845132 | 75586 | 16:43944562 | 75588 | 16:44032421 |
| 75608 | 16:44623635 | 75612 | 16:44826869 | 75613 | 16:44926372 | 75614 | 16:45026368 | 75616 | 16:45103219 |
| 75627 | 16:45512990 | 75632 | 16:45776402 | 75637 | 16:46002437 | 75779 | 16:49103242 | 75849 | 16:51054365 |
| 75850 | 16:51151596 | 75859 | 16:51446273 | 75867 | 16:51646308 | 75868 | 16:51740534 | 75944 | 16:53407745 |
| 75997 | 16:54730701 | 76093 | 16:56989855 | 76409 | 16:64087780 | 76503 | 16:65762070 | 76697 | 16:69928868 |
| 76757 | 16:71345017 | 76835 | 16:72851827 | 77085 | 16:77598464 | 77222 | 16:79857865 | 77229 | 16:80072357 |
| 77230 | 16:80138235 | 77232 | 16:80311616 | 77233 | 16:80396660 | 77235 | 16:80531024 | 77236 | 16:80630465 |
| 77239 | 16:80799166 | 77242 | 16:80953675 | 77252 | 17:259333 | 77263 | 17:564310 | 77274 | 17:973466 |
| 77278 | 17:1054010 | 77487 | 17:5568436 | 77620 | 17:8228129 | 77681 | 17:9733872 | 77735 | 17:10897059 |
| 77736 | 17:10938025 | 77737 | 17:11020572 | 77738 | 17:11094236 | 77755 | 17:11494094 | 77830 | 17:13277982 |
| 78070 | 17:18481630 | 78136 | 17:19808398 | 78137 | 17:19897663 | 78207 | 17:21967677 | 78230 | 17:22786786 |
| 78249 | 17:23511334 | 78255 | 17:23800711 | 78322 | 17:25936343 | 78401 | 17:27754738 | 78430 | 17:28426405 |
| 78441 | 17:28791960 | 78462 | 17:29447559 | 78487 | 17:30217269 | 78545 | 17:31620341 | 78567 | 17:32254712 |
| 78570 | 17:32296225 | 78667 | 17:34617927 | 78688 | 17:35304907 | 78690 | 17:35390256 | 78721 | 17:36229501 |
| 78734 | 17:36837955 | 78744 | 17:37134508 | 78795 | 17:38760200 | 78814 | 17:39443909 | 78936 | 17:41933427 |
| 78937 | 17:42031727 | 78986 | 17:42956052 | 78987 | 17:43050836 | 79027 | 17:44108368 | 79108 | 17:46082443 |
| 79178 | 17:47678534 | 79293 | 17:50425399 | 79393 | 17:52219542 | 79410 | 17:52792109 | 79481 | 17:54629610 |
| 79511 | 17:55298739 | 79535 | 17:55992314 | 79545 | 17:56246007 | 79980 | 17:64743637 | 80106 | 17:67144833 |
| 80130 | 17:67822583 | 80279 | 17:70683155 | 80280 | 17:70709345 | 80281 | 17:70709461 | 80291 | 17:71094967 |
| 80383 | 17:72831459 | 80395 | 18:95447 | 80396 | 18:189110 | 80398 | 18:367703 | 80400 | 18:514106 |
| 80908 | 18:9808096 | 80994 | 18:11474377 | 81022 | 18:12273578 | 81074 | 18:13692264 | 81098 | 18:14306814 |
| 81099 | 18:14400626 | 81103 | 18:14549787 | 81105 | 18:14605010 | 81106 | 18:14636355 | 81107 | 18:14731173 |
| 81108 | 18:14801380 | 81151 | 18:15872181 | 81153 | 18:15986314 | 81159 | 18:16182872 | 81377 | 18:20732386 |
| 81736 | 18:28718247 | 81811 | 18:30779424 | 81813 | 18:30921028 | 81837 | 18:31639943 | 81954 | 18:34144938 |
| 82045 | 18:36256440 | 82052 | 18:36555965 | 82152 | 18:38719188 | 82233 | 18:40460799 | 82285 | 18:41570886 |
| 82432 | 18:44652242 | 82443 | 18:44987987 | 82600 | 18:47190873 | 82671 | 18:48450267 | 82706 | 18:49271447 |
| 82707 | 18:49370683 | 82738 | 18:50083459 | 82759 | 18:50626419 | 82760 | 18:50725650 | 82837 | 18:52240322 |
| 82862 | 18:53044750 | 82869 | 18:53322848 | 82997 | 18:55515927 | 83109 | 18:56926638 | 83227 | 18:58451447 |
| 83237 | 18:58961238 | 83240 | 18:59153638 | 83249 | 18:59423487 | 83250 | 18:59502600 | 83251 | 18:59597971 |
| 83252 | 18:59640740 | 83253 | 18:59738484 | 83254 | 18:59752260 | 83255 | 18:59841292 | 83271 | 18:60187977 |
| 83273 | 18:60349514 | 83293 | 18:61084174 | 83294 | 18:61180054 | 83301 | 18:61392112 | 83302 | 18:61484398 |
| 83373 | 18:62547161 | 83374 | 18:62548072 | 83375 | 18:62548590 | 83376 | 18:62614389 | 83386 | 18:63006555 |
| 83387 | 18:63077361 | 83422 | 18:63662692 | 83588 | 19:57925 | 83591 | 19:305379 | 83596 | 19:417884 |
| 83597 | 19:509378 | 83599 | 19:577296 | 83600 | 19:668256 | 83629 | 19:1655405 | 83656 | 19:2558450 |
| 83665 | 19:2953866 | 83671 | 19:3120033 | 83677 | 19:3275802 | 83714 | 19:4397168 | 83980 | 19:9821822 |
| 84056 | 19:11385109 | 84253 | 19:15470245 | 84549 | 19:20561916 | 84554 | 19:20740986 | 84572 | 19:21295269 |
| 84638 | 19:22476147 | 84674 | 19:23274892 | 84679 | 19:23407987 | 84688 | 19:23703085 | 84976 | 19:27081319 |
| 84977 | 19:27125583 | 84978 | 19:27182483 | 84979 | 19:27275569 | 84986 | 19:27665926 | 85115 | 19:30350931 |
| 85176 | 19:31793582 | 85191 | 19:32153081 | 85511 | 19:37954850 | 85578 | 19:39940442 | 85649 | 19:41359770 |
| 85749 | 19:43284000 | 85860 | 19:45535788 | 85910 | 19:46199205 | 85919 | 19:46495194 | 85936 | 19:46990600 |
| 86031 | 19:48855388 | 86072 | 19:49706256 | 86111 | 19:50303597 | 86113 | 19:50431879 | 86114 | 19:50531654 |
| 86241 | 19:52131137 | 86250 | 19:52418467 | 86565 | 19:56677337 | 86621 | 19:57762258 | 86622 | 19:57849833 |
| 86961 | 20:98572 | 86962 | 20:194921 | 87107 | 20:2918386 | 87430 | 20:5583730 | 87593 | 20:8760244 |
| 87840 | 20:14544398 | 87842 | 20:14662366 | 87845 | 20:14842827 | 87846 | 20:14894317 | 87848 | 20:15043520 |
| 87850 | 20:15190994 | 87888 | 20:16233299 | 87915 | 20:16830331 | 87920 | 20:17017002 | 87956 | 20:18017776 |
| 87971 | 20:18426404 | 88037 | 20:20313521 | 88073 | 20:21317972 | 88231 | 20:23439914 | 88321 | 20:25028667 |
| 88322 | 20:25092607 | 88327 | 20:25369162 | 88338 | 20:25827366 | 88357 | 20:26444773 | 88371 | 20:26712661 |
| 88493 | 20:29517107 | 88527 | 20:30497850 | 88541 | 20:31003971 | 88598 | 20:32503045 | 88599 | 20:32522681 |
| 88600 | 20:32622352 | 88602 | 20:32778020 | 88695 | 20:34788751 | 88842 | 20:38032426 | 88884 | 20:39213757 |
| 89063 | 20:43010466 | 89065 | 20:43140540 | 89068 | 20:43254253 | 89073 | 20:43352423 | 89105 | 20:44220967 |
| 89127 | 20:44701792 | 89143 | 20:45276596 | 89180 | 20:46098985 | 89181 | 20:46185509 | 89205 | 20:47109652 |
| 89218 | 20:47365415 | 89224 | 20:47651501 | 89232 | 20:47954545 | 89237 | 20:48129247 | 89238 | 20:48218836 |
| 89257 | 20:48716255 | 89281 | 20:49326846 | 89283 | 20:49443154 | 89319 | 20:50486303 | 89360 | 20:51882151 |
| 89375 | 20:52355899 | 89376 | 20:52454269 | 89400 | 20:53180123 | 89438 | 20:54174787 | 89439 | 20:54272031 |
| 89441 | 20:54375180 | 89450 | 20:54601936 | 89464 | 20:55005757 | 89492 | 20:55642905 | 89496 | 20:55834687 |
| 89710 | 20:60274323 | 89746 | 20:61511139 | 89881 | 20:64307221 | 90177 | 20:70744162 | 90271 | 20:71587457 |
| 90278 | 20:71850045 | 90283 | 21:134737 | 90284 | 21:233193 | 90286 | 21:309646 | 90287 | 21:371966 |
| 90288 | 21:471326 | 90289 | 21:506222 | 90291 | 21:638478 | 90292 | 21:726326 | 90293 | 21:823686 |
| 90294 | 21:904065 | 90295 | 21:966923 | 90296 | 21:1061609 | 90297 | 21:1093204 | 90298 | 21:1137598 |
| 90300 | 21:1255004 | 90301 | 21:1331973 | 90302 | 21:1425976 | 90303 | 21:1485463 | 90304 | 21:1581245 |
| 90306 | 21:1694504 | 90308 | 21:1850823 | 90309 | 21:1924655 | 90323 | 21:2437601 | 90347 | 21:3138288 |
| 90349 | 21:3228959 | 90358 | 21:3544839 | 90359 | 21:3610217 | 90364 | 21:3820824 | 90406 | 21:4613245 |
| 90440 | 21:5697639 | 90462 | 21:6211650 | 90464 | 21:6330904 | 90478 | 21:6814441 | 90482 | 21:6933120 |
| 90484 | 21:7029661 | 90640 | 21:10179845 | 91036 | 21:19148704 | 91170 | 21:21513912 | 91265 | 21:23382552 |
| 91266 | 21:23474228 | 91273 | 21:23715679 | 91285 | 21:24111132 | 91308 | 21:24835888 | 91334 | 21:25749419 |
| 91346 | 21:26098112 | 91360 | 21:26431821 | 91386 | 21:27005517 | 91387 | 21:27103067 | 91433 | 21:28660586 |
| 91552 | 21:31691093 | 91568 | 21:32209086 | 91593 | 21:32883760 | 91594 | 21:32927345 | 91595 | 21:33019785 |
| 91724 | 21:34773624 | 91725 | 21:34862867 | 91834 | 21:37259953 | 91836 | 21:37337560 | 91837 | 21:37418367 |
| 91858 | 21:38022062 | 91859 | 21:38118482 | 91861 | 21:38269475 | 91894 | 21:39232808 | 91946 | 21:40644524 |
| 91947 | 21:40743815 | 91953 | 21:40912381 | 91994 | 21:41910649 | 92018 | 21:42440438 | 92023 | 21:42514187 |
| 92102 | 21:44357068 | 92140 | 21:44936824 | 92141 | 21:45020642 | 92143 | 21:45198066 | 92152 | 21:45602636 |
| 92165 | 21:46016604 | 92166 | 21:46114894 | 92169 | 21:46249176 | 92297 | 21:48618715 | 92405 | 21:51567065 |
| 92449 | 21:52692952 | 92450 | 21:52792942 | 92453 | 21:52960457 | 92620 | 21:56228179 | 92621 | 21:56313681 |
| 92623 | 21:56441767 | 92814 | 21:59309517 | 92815 | 21:59375264 | 93006 | 21:63376882 | 93229 | 21:67773042 |
| 93338 | 22:94579 | 93352 | 22:646933 | 93358 | 22:824901 | 93437 | 22:2467585 | 93677 | 22:7210800 |
| 93714 | 22:8230735 | 93734 | 22:8758516 | 93765 | 22:9634144 | 93849 | 22:11365346 | 93942 | 22:13343219 |
| 93966 | 22:14036315 | 93997 | 22:14787281 | 94092 | 22:17058957 | 94135 | 22:18197695 | 94162 | 22:18799270 |
| 94176 | 22:19116049 | 94364 | 22:24074772 | 94470 | 22:27069723 | 94481 | 22:27482704 | 94563 | 22:29220441 |
| 94595 | 22:30012007 | 94650 | 22:31491877 | 94657 | 22:31704814 | 95074 | 22:41624457 | 95174 | 22:43984117 |
| 95228 | 22:45235116 | 95236 | 22:45525829 | 95393 | 22:48902067 | 95902 | 22:58335431 | 95953 | 22:59355819 |
| 96035 | 23:278582 | 96037 | 23:430451 | 96042 | 23:626975 | 96071 | 23:1548151 | 96072 | 23:1635007 |
| 96178 | 23:3824609 | 96286 | 23:5931144 | 96463 | 23:9588241 | 96613 | 23:12692439 | 96648 | 23:13322760 |
| 96655 | 23:13471246 | 96656 | 23:13531584 | 97061 | 23:21612939 | 97062 | 23:21712441 | 97063 | 23:21811769 |
| 97086 | 23:22310386 | 97103 | 23:22816656 | 97117 | 23:23369505 | 97336 | 23:25965936 | 97337 | 23:26013871 |
| 97338 | 23:26203805 | 97339 | 23:26302825 | 97340 | 23:26400435 | 97341 | 23:26404617 | 97342 | 23:26417922 |
| 97343 | 23:26516930 | 97346 | 23:26688460 | 97347 | 23:26750001 | 97348 | 23:26836934 | 97638 | 23:30204175 |
| 97639 | 23:30205302 | 97640 | 23:30205793 | 97675 | 23:30951436 | 97705 | 23:31625189 | 97851 | 23:34829880 |
| 97852 | 23:34888702 | 97859 | 23:35057295 | 97860 | 23:35151436 | 97889 | 23:36152667 | 97963 | 23:38043115 |
| 98233 | 23:42608340 | 98516 | 23:48087560 | 98519 | 23:48274159 | 98668 | 23:50824134 | 98767 | 23:52402327 |
| 98771 | 24:201134 | 98932 | 24:3597084 | 98987 | 24:4929150 | 99002 | 24:5203768 | 99108 | 24:7965661 |
| 99109 | 24:8063828 | 99131 | 24:8583177 | 99304 | 24:10935542 | 99358 | 24:12240445 | 99425 | 24:13974458 |
| 99478 | 24:15604573 | 99486 | 24:15989036 | 99487 | 24:16079054 | 99561 | 24:18229831 | 99572 | 24:18541933 |
| 99573 | 24:18640412 | 99606 | 24:19585603 | 99618 | 24:19942905 | 99624 | 24:20173407 | 99688 | 24:21131115 |
| 99764 | 24:22214747 | 99765 | 24:22312329 | 99816 | 24:23615391 | 99822 | 24:23975968 | 99826 | 24:24145037 |
| 99942 | 24:26860326 | 100052 | 24:29671134 | 100105 | 24:30883521 | 100106 | 24:30966522 | 100219 | 24:33423144 |
| 100223 | 24:33544520 | 100339 | 24:36290089 | 100345 | 24:36532147 | 100399 | 24:38166045 | 100405 | 24:38422210 |
| 100411 | 24:38722958 | 100472 | 24:40089318 | 100473 | 24:40188306 | 100548 | 24:41297881 | 100621 | 24:42939236 |
| 100630 | 24:43395685 | 100644 | 24:43924798 | 100645 | 24:44019504 | 100798 | 24:46654829 | 100855 | 24:48033977 |
| 100863 | 24:48144296 | 100879 | 24:48567652 | 100883 | 24:48858324 | 101019 | 24:52201248 | 101051 | 24:52902393 |
| 101118 | 24:54423570 | 101185 | 24:55890705 | 101203 | 24:56201929 | 101491 | 24:61932447 | 101503 | 24:62180714 |
| 101504 | 24:62277921 | 101505 | 25:94971 | 101613 | 25:2097812 | 101744 | 25:3066063 | 101992 | 25:7620341 |
| 102020 | 25:8255600 | 102049 | 25:8952928 | 102054 | 25:9079254 | 102288 | 25:14296254 | 102469 | 25:18216204 |
| 102662 | 25:22863656 | 102693 | 25:23717883 | 102857 | 25:26775859 | 103041 | 25:30451801 | 103048 | 25:30672143 |
| 103152 | 25:32745193 | 103607 | 25:40515689 | 103707 | 26:71749 | 103708 | 26:134754 | 103709 | 26:202644 |
| 103710 | 26:292215 | 103711 | 26:332815 | 103712 | 26:418885 | 103713 | 26:467022 | 103714 | 26:552651 |
| 103715 | 26:648778 | 103720 | 26:841636 | 103721 | 26:933342 | 103740 | 26:1410312 | 103765 | 26:2231655 |
| 103788 | 26:2771137 | 103883 | 26:4804367 | 103903 | 26:5316048 | 103915 | 26:5782436 | 104029 | 26:8899248 |
| 104047 | 26:9422315 | 104053 | 26:9629233 | 104130 | 26:11253125 | 104132 | 26:11345317 | 104133 | 26:11428247 |
| 104158 | 26:11886123 | 104260 | 26:14376252 | 104287 | 26:15092211 | 104288 | 26:15180609 | 104328 | 26:16278407 |
| 104330 | 26:16409629 | 104334 | 26:16616504 | 104489 | 26:19837144 | 104494 | 26:20063209 | 104500 | 26:20330629 |
| 104526 | 26:21059250 | 104617 | 26:23492556 | 104628 | 26:23837564 | 104631 | 26:23968846 | 104811 | 26:27340392 |
| 104865 | 26:28704657 | 104921 | 26:30296052 | 104930 | 26:30555507 | 104967 | 26:31506273 | 105102 | 26:34494728 |
| 105234 | 26:37659896 | 105300 | 26:39301430 | 105325 | 26:39903212 | 105389 | 26:41736203 | 105425 | 26:42498087 |
| 105426 | 26:42598014 | 105453 | 26:43157974 | 105522 | 26:44983809 | 105581 | 26:45965280 | 105755 | 26:49691984 |
| 105779 | 26:50189494 | 105780 | 26:50197785 | 105781 | 26:50203962 | 105782 | 26:50261536 | 105783 | 26:50350367 |
| 105784 | 26:50350409 | 105785 | 26:50414892 | 105855 | 27:99932 | 105856 | 27:181641 | 105857 | 27:250836 |
| 105858 | 27:316731 | 105859 | 27:394123 | 105860 | 27:443903 | 105864 | 27:543122 | 105866 | 27:642666 |
| 105867 | 27:739223 | 105868 | 27:838134 | 105869 | 27:928860 | 105870 | 27:1021674 | 105871 | 27:1119724 |
| 105923 | 27:1865958 | 105994 | 27:3535767 | 105999 | 27:3755867 | 106117 | 27:6215443 | 106119 | 27:6337782 |
| 106121 | 27:6434403 | 106122 | 27:6434442 | 106123 | 27:6621177 | 106124 | 27:6714941 | 106125 | 27:6754631 |
| 106126 | 27:6761335 | 106127 | 27:6952355 | 106128 | 27:6962879 | 106129 | 27:6980835 | 106130 | 27:6994129 |
| 106250 | 27:9301634 | 106268 | 27:9835607 | 106279 | 27:10239566 | 106329 | 27:11805253 | 106337 | 27:12112133 |
| 106347 | 27:12322130 | 106348 | 27:12386638 | 106458 | 27:14207575 | 106659 | 27:18065646 | 106660 | 27:18151157 |
| 106710 | 27:19373108 | 106765 | 27:20532211 | 106773 | 27:20833561 | 106823 | 27:22102480 | 106827 | 27:22221678 |
| 106829 | 27:22350322 | 106845 | 27:22785446 | 106999 | 27:26185311 | 107112 | 27:28416757 | 107186 | 27:30124180 |
| 107366 | 27:33574896 | 107379 | 27:34012998 | 107476 | 27:35694726 | 107536 | 27:36991890 | 107623 | 27:38609999 |
| 107654 | 27:39285504 | 107891 | 27:44800138 | 107919 | 27:45569924 | 107920 | 28:39225 | 107921 | 28:138659 |
| 107922 | 28:231989 | 107923 | 28:285448 | 107924 | 28:328039 | 107925 | 28:386583 | 107929 | 28:563052 |
| 107930 | 28:662354 | 107934 | 28:794289 | 107935 | 28:893452 | 108018 | 28:2643580 | 108026 | 28:2904232 |
| 108027 | 28:2999388 | 108032 | 28:3161546 | 108033 | 28:3253315 | 108036 | 28:3405359 | 108042 | 28:3759573 |
| 108043 | 28:3787485 | 108044 | 28:3885321 | 108048 | 28:4073833 | 108049 | 28:4163374 | 108050 | 28:4253300 |
| 108051 | 28:4338728 | 108053 | 28:4513285 | 108069 | 28:5004251 | 108185 | 28:7591484 | 108274 | 28:9724722 |
| 108313 | 28:10660380 | 108327 | 28:11111391 | 108329 | 28:11222161 | 108430 | 28:13814734 | 108543 | 28:16313184 |
| 108613 | 28:17683321 | 108639 | 28:18306254 | 108745 | 28:21104115 | 108771 | 28:22035988 | 108799 | 28:22784794 |
| 108801 | 28:22871308 | 108802 | 28:22889009 | 108844 | 28:23719431 | 108845 | 28:23801384 | 109021 | 28:27215263 |
| 109182 | 28:30404928 | 109336 | 28:33475488 | 109380 | 28:34421752 | 109439 | 28:35588714 | 109492 | 28:36727428 |
| 109624 | 28:39439768 | 109625 | 28:39494932 | 109633 | 28:39750515 | 109936 | 28:44515897 | 110008 | 29:178851 |
| 110009 | 29:265250 | 110013 | 29:607202 | 110077 | 29:1956188 | 110081 | 29:2091012 | 110237 | 29:5374583 |
| 110238 | 29:5456512 | 110239 | 29:5547355 | 110240 | 29:5627071 | 110241 | 29:5698810 | 110284 | 29:7011954 |
| 110333 | 29:8334191 | 110338 | 29:8529992 | 110547 | 29:13277373 | 110553 | 29:13442800 | 110584 | 29:14334295 |
| 110592 | 29:14552146 | 110661 | 29:16276745 | 110732 | 29:18298255 | 110742 | 29:18821433 | 110748 | 29:19176738 |
| 110756 | 29:19577395 | 110814 | 29:20938903 | 110816 | 29:21062969 | 110847 | 29:21741949 | 111027 | 29:26286827 |
| 111040 | 29:26637610 | 111042 | 29:26812835 | 111055 | 29:27123234 | 111057 | 29:27286001 | 111060 | 29:27336935 |
| 111061 | 29:27426724 | 111385 | 29:34145956 | 111519 | 29:36681488 | 111565 | 29:37610625 | 111572 | 29:37814400 |
| 111573 | 29:37903323 | 111574 | 29:37975002 | 111575 | 29:38065526 | 111576 | 29:38161198 | 111577 | 29:38251275 |
| 111578 | 29:38348064 | 111579 | 29:38414602 | 111580 | 29:38450767 | 111581 | 29:38467768 | 111582 | 29:38556318 |
| 111583 | 29:38649308 | 111586 | 29:38752213 | 111587 | 29:38847684 | 111588 | 29:38917900 | 111589 | 29:38952660 |
| 111590 | 29:38993981 | 111591 | 29:39083787 | 111593 | 29:39205805 | 111728 | 29:41919049 | 112146 | 29:50420995 |
| 112179 | 29:50977672 | 112180 | 29:51071432 | | | | | | |

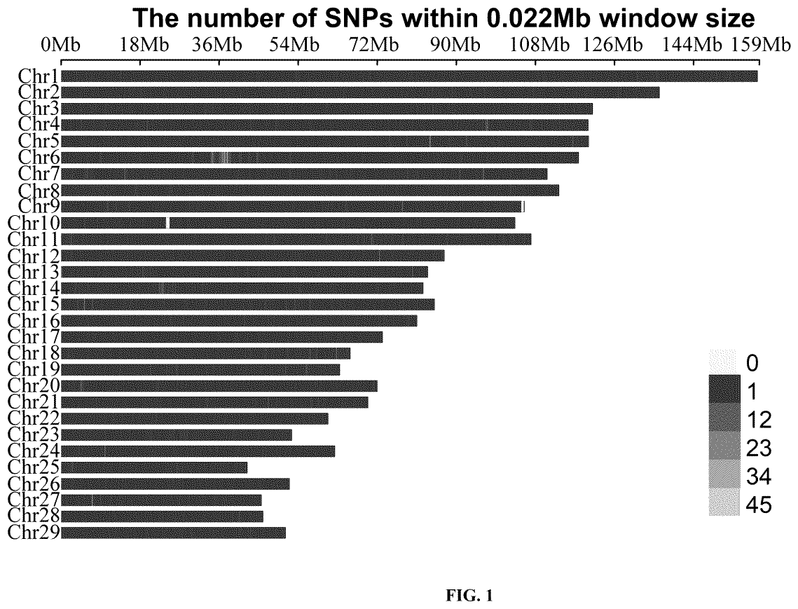

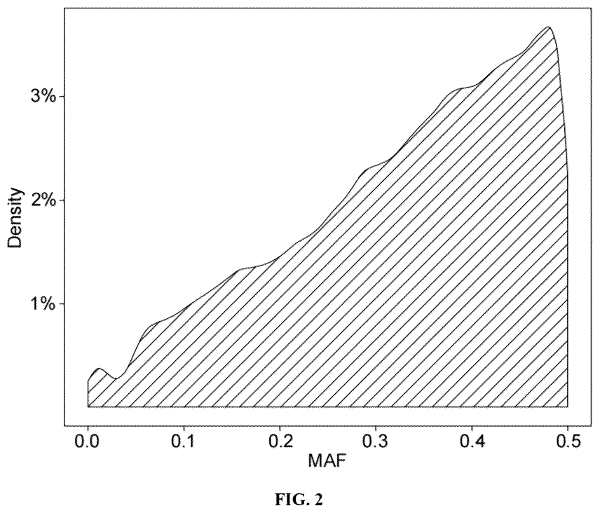

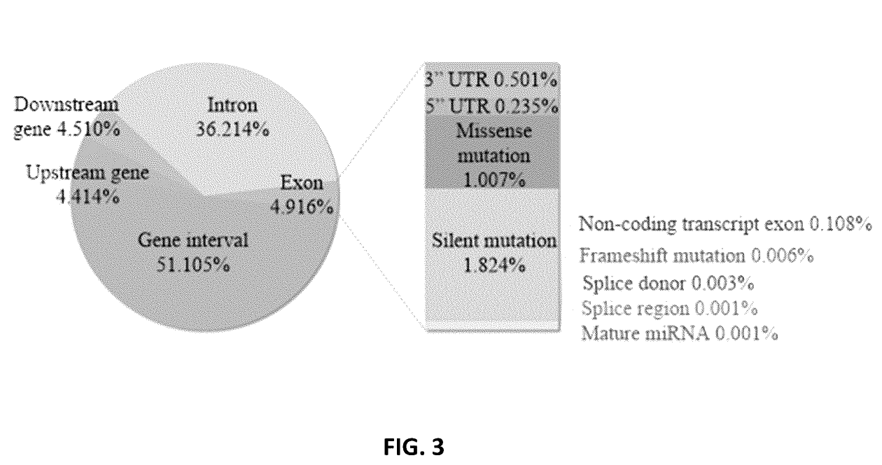

The present disclosure provided a molecular marker combination for whole-genome breeding of Huaxi cattle, where 112,180 genetic variations were evenly covered on the whole-genome (FIG. 1). The trait-associated markers of Huaxi cattle involved seven categories of growth, fattening, carcass, meat quality, reproduction, disease, and health, including a total of 129 traits containing 13,581 important functional markers. SNP polymorphism performed well in the Huaxi cattle population with an average MAF was 0.33 (FIG. 2). Through the optimal design of multi-level omics such as resequencing, transcriptome, exome, and epigenomics, the newly discovered trait-specific functional markers of Huaxi cattle were selected, and the genome functional region variations had a high proportion (FIG. 3).

### Example 2

Statistics and Use of Heritability of Various Traits of Huaxi Cattle Based on Cattle110K Chip

The Cattle110K chip provided by the present disclosure was used to detect the genotypes of 1,233 individuals in the Huaxi cattle resource population. For the important economic traits of Huaxi cattle, a REML method of Asreml software was used to estimate the heritability of each trait based on two different SNP numbers of Cattle110K chip and Illumina BovineHD (770K) beadchip. Through the heritability estimation of traits such as slaughter, meat quality, growth, and GCBI (Table 7), 110K and 770K showed stable heritability at two different SNP numbers, proving the reliability of the Cattle110K chip for GS.

TABLE 7
Heritability estimation of the important
economic traits of Huaxi cattle traits

| Classification | | | Illumina |
|---|---|---|---|
| of traits | Trait indicators | Cattle110K | BovineHD |
| Slaughter trait | Lean meat weight | 0.519 | 0.468 |
| | Lean meat percentage | 0.435 | 0.418 |
| | Carcass length | 0.371 | 0.377 |
| Meat quality | Ribeye area (12 ribs) | 0.454 | 0.411 |
| trait | Shear force | 0.453 | 0.472 |
| Growth trait | Heart girth (12 months old) | 0.446 | 0.453 |
| | Body length (12 months old) | 0.327 | 0.334 |
| GCBI trait | Weaning weight | 0.470 | 0.470 |
| | Average daily gain during | 0.743 | 0.738 |
| | fattening period | | |
| | Carcass weight | 0.629 | 0.624 |
| | Dressing percentage | 0.515 | 0.533 |
| | Calving ease | 0.138 | 0.134 |

### Example 3

Genetic Background Analysis of Huaxi Cattle Based on Cattle110K Genotyping Results

In order to verify use of the GS breeding chip of Huaxi cattle in the genotype identification of Huaxi cattle population, the Cattle110K chip of the present disclosure was used to evaluate the genetic diversity of a total of 9 cattle breeds (populations). The breeds (populations) included Huaxi cattle, Mongolian cattle, Sanhe cattle, Charolais cattle, Australian Simmental cattle, Canadian Simmental cattle, American Simmental cattle, German Simmental cattle, and French Simmental cattle. The source and number of samples of each breed were shown in Table 8.

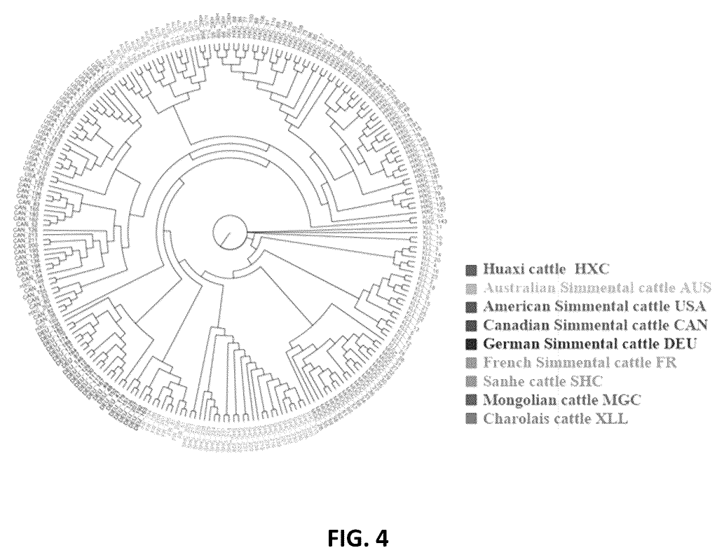

Cluster analysis was conducted by Neighbor-joining method, and an individual-based phylogenetic tree was constructed. As shown in FIG. 4, each breed clustered clearly on the phylogenetic tree, and the classification was clear. The results showed that the populations of Huaxi cattle could obviously gather together to form an independent branch, and the population genetic consistency was relatively desirable. The evolutionary relationship between Huaxi cattle and other breeds was as follows: the Australian, American, and Canadian lines of Simmental cattle for meat, followed by the German and French lines of Simmental cattle for both milk and meat, and finally the Sanhe cattle, Mongolian cattle, and Charolais cattle.

By calculating the Nei's genetic distance and F-statistic (Fst) value or fixation index between each population, the degree of genetic difference between Huaxi cattle and other cattle breeds at the population level was analyzed. The results were shown in Table 9. The Nei distance between the 9 populations ranged from 0.0734 to 0.2252, where Huaxi cattle had the closest genetic distance (0.0734) to Australian Simmental cattle, and had a farther genetic distance (0.1801) to Mongolian cattle. The Fst values of the 9 populations ranged from 0.0251 to 0.1231, where Huaxi cattle had the least degree of differentiation (0.0323) from Australian Simmental cattle, and had a greater degree of differentiation from Mongolian cattle and Sanhe cattle.

TABLE 8
List of cattle breeds (populations)

| | Breed | | |
|---|---|---|---|
| SN | (Population) | Number | Sample source |
| 1 | Huaxi cattle | 67 | Huaxi Cattle resource group in |
| | | | Wulagai, Inner Mongolia |
| 2 | Mongolian cattle | 20 | Lvsen Cattle Farm in Alxa Left |
| | | | Banner, Inner Mongolia |
| 3 | Sanhe cattle | 25 | Xier-tala Farm and Ranch in |
| | | | Hulunbuir State Farm, Inner Mongolia |
| 4 | Charolais cattle | 25 | National Germplasm Center of |
| | | | Domestic Animal Resources in China |
| 5 | Australian | 21 | National Germplasm Center of |
| | Simmental cattle | | Domestic Animal Resources in China |
| 6 | Canadian | 25 | National Germplasm Center of |
| | Simmental cattle | | Domestic Animal Resources in China |
| 7 | American | 25 | National Germplasm Center of |
| | Simmental cattle | | Domestic Animal Resources in China |
| 8 | German | 25 | National Germplasm Center of |
| | Simmental cattle | | Domestic Animal Resources in China |
| 9 | French | 8 | National Germplasm Center of |
| | Simmental cattle | | Domestic Animal Resources in China |

TABLE 9
Fst value (lower left) and Nei distance (upper right) between Huaxi cattle and other cattle breeds (populations)

| | Nei | | | | | | | | |
|---|---|---|---|---|---|---|---|---|---|
| | | | | Australian | Canadian | American | German | French | |
| | Mongolian | Sanhe | Charolais | Simmental | Simmental | Simmental | Simmental | Simmental | Huaxi |
| Fst | cattle | cattle | cattle | cattle | cattle | cattle | cattle | cattle | cattle |
| Mongolian cattle | — | 0.1589 | 0.1337 | 0.1760 | 0.2005 | 0.2252 | 0.1738 | 0.2139 | 0.1801 |
| Sanhe cattle | 0.0676 | — | 0.1078 | 0.1389 | 0.1643 | 0.1919 | 0.1300 | 0.1623 | 0.1406 |
| Charolais cattle | 0.0475 | 0.0451 | — | 0.1170 | 0.1436 | 0.1728 | 0.1121 | 0.1495 | 0.1191 |
| Australian Simmental cattle | 0.0790 | 0.0656 | 0.0488 | — | 0.0990 | 0.1306 | 0.0830 | 0.1292 | 0.0734 |
| Canadian Simmental cattle | 0.1019 | 0.0874 | 0.0724 | 0.0435 | — | 0.1449 | 0.1033 | 0.1487 | 0.1018 |
| American Simmental cattle | 0.1231 | 0.1073 | 0.0945 | 0.0687 | 0.0812 | — | 0.1345 | 0.1819 | 0.1370 |
| German Simmental cattle | 0.0765 | 0.0610 | 0.0470 | 0.0267 | 0.0468 | 0.0701 | — | 0.1177 | 0.0867 |
| French Simmental cattle | 0.0717 | 0.0544 | 0.0416 | 0.0315 | 0.0553 | 0.0863 | 0.0252 | — | 0.1327 |
| Huaxi cattle | 0.0985 | 0.0823 | 0.0674 | 0.0323 | 0.0548 | 0.0779 | 0.0436 | 0.0495 | — |

### Example 4

Evaluation of Imputation Accuracy of Cattle110K

In the Huaxi cattle resource population, 414 individuals with Cattle110K chip genotyping data were selected as a verification group, and 4,203 individuals with Illumina BovineHD beadchip genotyping data were selected as a reference group. Through the Beagle v5.0 software, SNP genotypes of Cattle110K chip was imputed to marker density of Illumina BovineHD beadchip. The genotype concordance and genotype correlation coefficient were used as evaluation criteria for the accuracy of genotype imputation. On one hand, the percentage of correctly imputed genotypes was calculated for each individual, resulting in the average genotype concordance of 0.986 for the 414 individuals. Among them, 408 individuals had a percentage greater than 0.950, accounting for 98.55% of the total individuals. On the other hand, the correlation coefficient between the imputed and true genotype was calculated for each individual. The average genotype correlation coefficient of 414 individuals was Among them, 396 individuals had a coefficient greater than 0.950, accounting for 95.65% of the total individuals.

### Example 5

GWAS of the Important Economic Traits of Huaxi Cattle Based on Cattle110K Chip

The Cattle110K chip provided by the present disclosure was used to detect the genotypes of 1,233 individuals in the Huaxi cattle resource population. The quality control standards of the genotype data were as follows: a SNP call rate was greater than 90%, a MAF was greater than 0.05, and a P value of the HWE testing was less than 1×10⁻⁶. After quality control, a total of 104,575 SNPs were remaining for GWAS on the important economic traits of Huaxi cattle, including hip height (weaning), abdominal circumference (6 months old), slaughter weight, dressing percentage, carcass length, and marbling score.

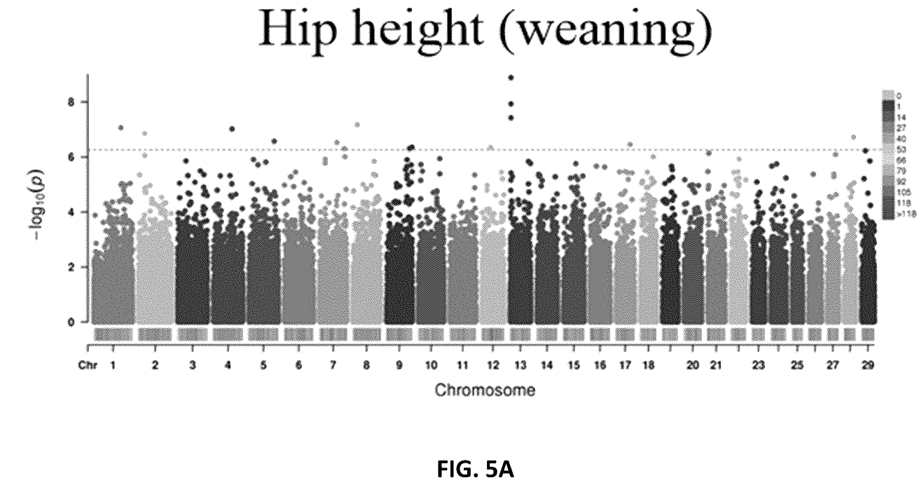

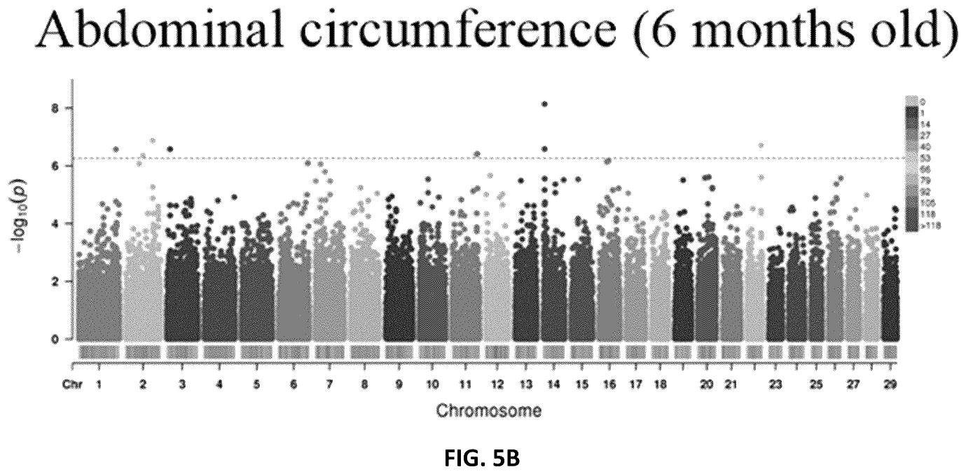

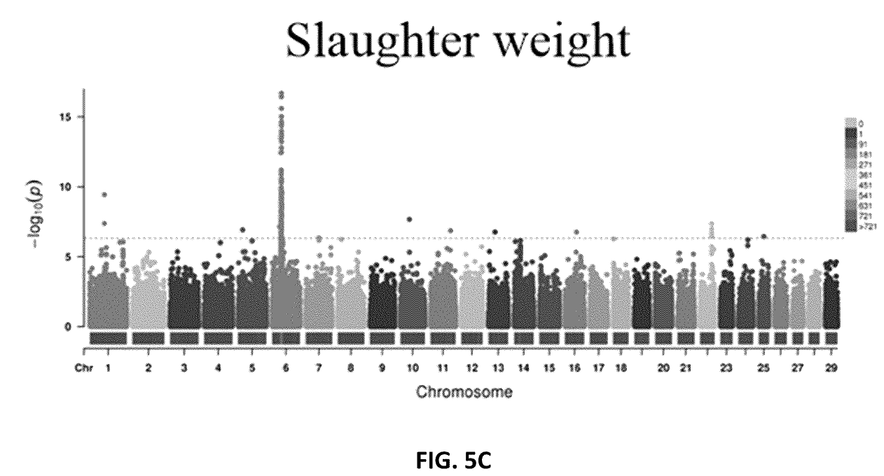

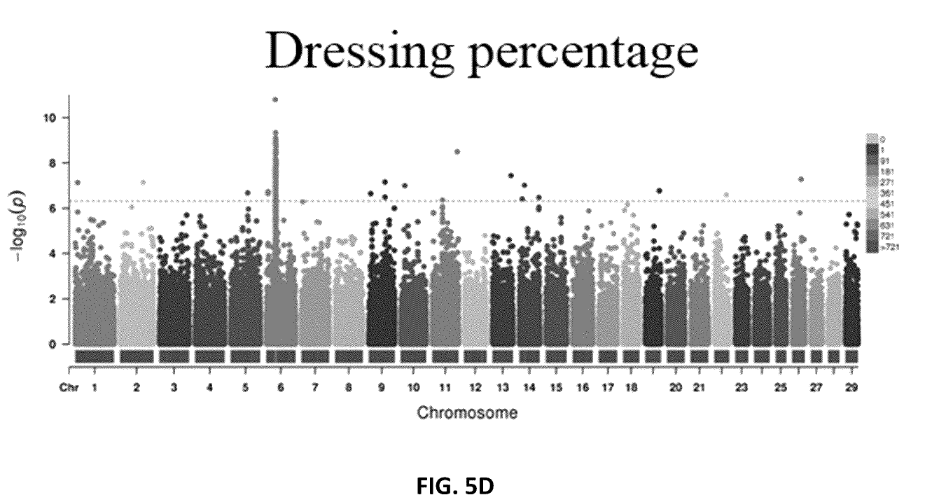

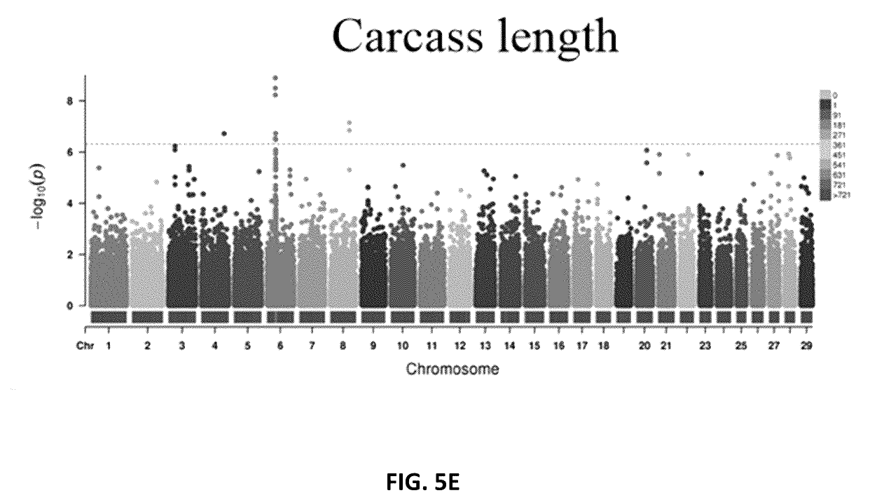

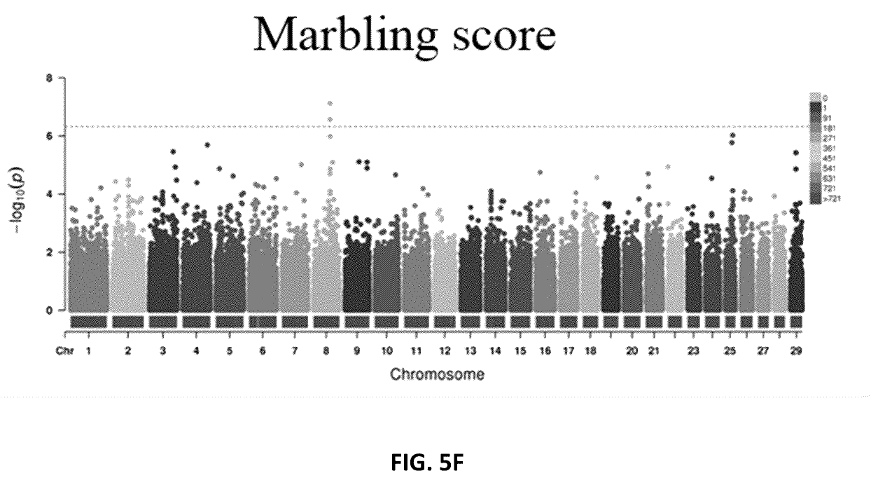

A general linear model was applied for the GWAS analysis. P value of independent test was 4.78×10⁻⁷ (0.05/104575) as a significance threshold of the whole-genome (FIGS. 5A-F). The results showed that significant SNPs associated with the above six important economic traits were found. Especially, there was a region on chromosome 6 that was significantly associated with the body shape and body weight in the GWAS analysis on slaughter weight, dressing percentage, and carcass length of Huaxi cattle. Therefore, the genotyping of the Huaxi cattle 110K chip of the present disclosure could obtain more accurate GWAS results.

### Example 6

Use of GS Breeding Based on Cattle110K Chip

The Cattle110K chip provided by the present disclosure was used to conduct genome-wide genetic evaluation on Huaxi cattle population. A specific method included: (1) genotyping was conducted on 1,233 individuals in the Huaxi cattle resource group established in a pasture of the Wulagai management area of Xilin-Gol League in Inner Mongolia. (2) Genotype quality control was conducted at a SNP call rate of greater than 90%, a MAF of greater than 0.05, and a P value of HWE testing of less than 1×10⁻⁶. (3) GEBV prediction of 7 important economic traits including slaughter, meat quality, and body size, and 5 GCBI traits (calving ease, weaning weight, average daily gain during fattening period, carcass weight, and dressing percentage) was estimated using GBLUP and BayesB methods, separately. (4) An accuracy of GEBV was calculated by 5-fold cross-validation, and the results of the Cattle110K chip and Illumina BovineHD beadchip were compared. The calculation results were shown in Table 10.

Regarding the slaughter traits such as lean meat weight, dressing percentage, and carcass length, the accuracy of GEBV calculated by GBLUP and BayesB methods was relatively consistent in Cattle110K chip provided by the present disclosure and Illumina BovineHD beadchip. Specifically, in the lean meat weight and dressing percentage traits, the accuracy of Cattle110K calculated by the GBLUP and BayesB was higher than that of Illumina BovineHD. The accuracy of lean meat weight increased by 5.81% and 7.11%, respectively, and the accuracy of dressing percentage increased by 6.86% and 3.11%, respectively. In terms of carcass length trait, the accuracy of Cattle110K calculated by the GBLUP and BayesB was slightly lower than that of Illumina BovineHD, with a decrease of 1.58% and 7.73%, respectively.

For the two meat traits of shear force and ribeye area, the GEBV results of Cattle110K chip and Illumina BovineHD beadchip with the two analysis methods were slightly different. Specifically, in the shear force trait, the accuracy of Cattle110K calculated by the GBLUP and BayesB was higher than that of Illumina BovineHD, which increased by 2.71% and 9.60%, respectively. In the ribeye area trait, the GBLUP result of Cattle110K was 2.12% higher than that of Illumina BovineHD, while the BayesB result showed a decrease of 0.21%.

For the two body size traits of body length and heart girth at 12 months of age, the GEBV results of Cattle110K chip and Illumina BovineHD beadchip with the two analysis methods were slightly different. Specifically, in the body length trait, the GBLUP result of Cattle110K was 0.18% higher than that of Illumina BovineHD, while the BayesB result showed a decrease of 8.87%. In the terms of heart girth trait, the accuracy results of GEBV calculated by GBLUP and BayesB methods using Cattle110K chip were lower than those of Illumina BovineHD beadchip, with a decrease of 0.80% and 5.55%, respectively.

For the five GCBI traits, including weaning weight, average daily gain during fattening period, carcass weight, dressing percentage, and calving ease, the GEBV results of Cattle110K chip and Illumina BovineHD beadchip with the two analysis methods were slightly different. Specifically, in the weaning weight trait, the GBLUP result of Cattle110K was 0.20% lower than that of Illumina BovineHD, while the BayesB result showed an increase of 3.22%. In the average daily gain trait during fattening period, the GBLUP result of Cattle110K was 0.31% higher than that of Illumina BovineHD, while the BayesB result showed a decrease of 1.65%. In the carcass weight trait, the GBLUP result of Cattle110K was 3.97% higher than that of Illumina BovineHD, while the BayesB result showed a decrease of 2.84%. In the dressing percentage trait, the accuracy results of Cattle110K were improved in both methods by 2.91% and 1.94%, respectively. In the calving ease, the GBLUP result of Cattle110K was 1.31% lower than that of Illumina BovineHD, while the BayesB results showed an increase of 1.71%.

From the above results, although the number of markers used by the Cattle110K chip provided by the present disclosure was lower than that of the Illumina BovineHD chip (770K), the results of genetic evaluation of traits such as slaughter, meat quality, growth and development, and GCBI traits showed that the accuracy of GEBV prediction of the Cattle110K chip was highly consistent with that of Illumina BovineHD beadchip. Compared with the accuracy results estimated by Illumina BovineHD, the accuracy of Cattle110K calculated by GBLUP and BayesB was improved or decreased by not more than 10%. This indicated that the SNPs selected in the Cattle110K chip of Huaxi cattle could provide informative and accurate data for GS, and the evaluation results of many traits were better, thereby contributing the genetic improvement in GS breeding of Huaxi cattle.

TABLE 10
The accuracy of the genomic estimated breeding value (GEBV) for the important economic traits

| Classification of | | GBLUP | | BayesB | |
|---|---|---|---|---|---|
| traits | Trait | Cattle110K | 770K | Cattle110K | 770K |
| Slaughter trait | Lean meat weight | 0.510 | 0.482 | 0.512 | 0.478 |
| | Lean meat percentage | 0.452 | 0.423 | 0.431 | 0.418 |
| | Carcass length | 0.374 | 0.380 | 0.358 | 0.388 |
| Meat quality trait | Ribeye area (12 ribs) | 0.482 | 0.472 | 0.471 | 0.472 |
| | Shear force | 0.492 | 0.479 | 0.525 | 0.479 |
| Body size trait | Heart girth (12 months old) | 0.544 | 0.543 | 0.493 | 0.541 |
| | Body length (12 months old) | 0.617 | 0.622 | 0.596 | 0.631 |
| GCBI trait | Weaning weight | 0.505 | 0.506 | 0.513 | 0.497 |
| | Average daily gain during fattening period | 0.653 | 0.651 | 0.654 | 0.665 |
| | Carcass weight | 0.603 | 0.580 | 0.606 | 0.624 |
| | Dressing percentage | 0.530 | 0.515 | 0.525 | 0.515 |
| | Calving ease | 0.528 | 0.535 | 0.536 | 0.527 |

The above are merely preferred implementations of the present disclosure. It should be noted that several improvements and modifications may further be made by a person of ordinary skill in the art without departing from the principle of the present disclosure, and such improvements and modifications should also be deemed as falling within the protection scope of the present disclosure.

1-10. (canceled)

11. A method for detection of genotyping of the Huaxi cattle using a genome-wide breeding chip of Huaxi cattle, wherein the genome-wide breeding chip of Huaxi cattle comprises a molecular marker combination;
wherein the molecular marker combination comprises seven marker panels; a first marker panel comprising 7,221 SNPs; a second marker panel comprising 22,937 SNPs and 1 Indel; a third marker panel comprising 249 SNPs; a fourth marker panel comprising 3,907 SNPs and 2 Indels; a fifth marker panel comprising 6,190 SNPs; a sixth marker panel comprising a total of 74,098 markers verified in a Huaxi cattle population from Illumina BovineHD chip and Neogen GGP Bovine 100K chip; a seventh marker panel comprising 2,617 SNPs; and an ARS-UCD 1.2/bosTau9 version is used as a reference genome of cattle;
the sites of the first marker panel are shown in the following table:
TABLE 11

| NO. | Position | NO. | Position | NO. | Position | NO. | Position | NO. | Position |
|---|---|---|---|---|---|---|---|---|---|
| 27 | 1:1073001 | 40 | 1:1271108 | 124 | 1:3014460 | 164 | 1:3598559 | 213 | 1:4312806 |
| 216 | 1:4341656 | 257 | 1:5479286 | 286 | 1:6042789 | 314 | 1:6638827 | 324 | 1:6809931 |
| 386 | 1:8222113 | 399 | 1:8645362 | 407 | 1:8747963 | 480 | 1:10381445 | 484 | 1:10442662 |
| 547 | 1:12065998 | 575 | 1:12850062 | 586 | 1:13291614 | 593 | 1:13478803 | 594 | 1:13478804 |
| 595 | 1:13478805 | 600 | 1:13618910 | 602 | 1:13649839 | 603 | 1:13650350 | 604 | 1:13650573 |
| 605 | 1:13650578 | 606 | 1:13657524 | 607 | 1:13657759 | 608 | 1:13657853 | 609 | 1:13659104 |
| 610 | 1:13659735 | 611 | 1:13660313 | 612 | 1:13662505 | 613 | 1:13662879 | 614 | 1:13663313 |
| 615 | 1:13663369 | 632 | 1:14229816 | 635 | 1:14367624 | 640 | 1:14444223 | 646 | 1:14671195 |
| 675 | 1:15289219 | 732 | 1:17104389 | 745 | 1:17433162 | 761 | 1:18058788 | 779 | 1:18612790 |
| 803 | 1:19491923 | 807 | 1:19546032 | 831 | 1:20249479 | 855 | 1:20731028 | 866 | 1:21118209 |
| 901 | 1:22054771 | 936 | 1:23011477 | 954 | 1:23715328 | 957 | 1:23772692 | 974 | 1:24145046 |
| 1014 | 1:25285394 | 1015 | 1:25305166 | 1018 | 1:25448644 | 1038 | 1:25974291 | 1075 | 1:26927446 |
| 1089 | 1:27357800 | 1132 | 1:29040826 | 1142 | 1:29268481 | 1173 | 1:30001509 | 1176 | 1:30051285 |
| 1177 | 1:30052889 | 1206 | 1:31087002 | 1207 | 1:31090461 | 1211 | 1:31197827 | 1243 | 1:32461661 |
| 1255 | 1:32885018 | 1263 | 1:33160247 | 1384 | 1:36327051 | 1385 | 1:36335560 | 1404 | 1:36719216 |
| 1410 | 1:36920529 | 1412 | 1:36984896 | 1458 | 1:38181926 | 1480 | 1:38770057 | 1483 | 1:38899433 |
| 1495 | 1:39263861 | 1539 | 1:40637359 | 1546 | 1:40754498 | 1571 | 1:41666745 | 1617 | 1:42491489 |
| 1667 | 1:43750606 | 1687 | 1:44125197 | 1691 | 1:44260960 | 1710 | 1:44679137 | 1713 | 1:44805494 |
| 1719 | 1:44925165 | 1720 | 1:44925173 | 1721 | 1:44925749 | 1722 | 1:44927653 | 1723 | 1:44928339 |
| 1724 | 1:44928350 | 1725 | 1:44928489 | 1726 | 1:44929514 | 1727 | 1:44929625 | 1728 | 1:44929973 |
| 1729 | 1:44931081 | 1730 | 1:44931463 | 1753 | 1:45241871 | 1820 | 1:47384886 | 1828 | 1:47617677 |
| 1841 | 1:48116097 | 1963 | 1:52181284 | 1995 | 1:53016532 | 2003 | 1:53237486 | 2005 | 1:53252387 |
| 2006 | 1:53260493 | 2009 | 1:53326573 | 2013 | 1:53415161 | 2016 | 1:53539734 | 2030 | 1:53652726 |
| 2044 | 1:53770698 | 2051 | 1:54027097 | 2052 | 1:54039879 | 2053 | 1:54067827 | 2054 | 1:54068465 |
| 2062 | 1:54170560 | 2063 | 1:54175315 | 2073 | 1:54409914 | 2077 | 1:54504340 | 2078 | 1:54507288 |
| 2094 | 1:54985100 | 2100 | 1:55132890 | 2113 | 1:55602206 | 2115 | 1:55658614 | 2130 | 1:56173438 |
| 2133 | 1:56288769 | 2135 | 1:56366295 | 2136 | 1:56367389 | 2137 | 1:56367471 | 2140 | 1:56429824 |
| 2141 | 1:56439077 | 2151 | 1:56684792 | 2155 | 1:56767210 | 2164 | 1:56950851 | 2239 | 1:58810751 |
| 2254 | 1:59120478 | 2289 | 1:60052451 | 2299 | 1:60321205 | 2305 | 1:60534555 | 2314 | 1:60643904 |
| 2322 | 1:60871605 | 2331 | 1:61098681 | 2344 | 1:61320487 | 2345 | 1:61335565 | 2350 | 1:61476653 |
| 2374 | 1:62211191 | 2410 | 1:62916376 | 2411 | 1:62918132 | 2415 | 1:63017753 | 2416 | 1:63029817 |
| 2417 | 1:63033101 | 2418 | 1:63035068 | 2419 | 1:63037531 | 2420 | 1:63038112 | 2421 | 1:63038183 |
| 2422 | 1:63050131 | 2424 | 1:63119198 | 2425 | 1:63125457 | 2426 | 1:63128635 | 2427 | 1:63130293 |
| 2436 | 1:63393320 | 2450 | 1:63809663 | 2531 | 1:65592899 | 2574 | 1:66873010 | 2577 | 1:67103986 |
| 2584 | 1:67236736 | 2585 | 1:67260968 | 2636 | 1:68655539 | 2642 | 1:68743594 | 2664 | 1:68997540 |
| 2707 | 1:69843446 | 2722 | 1:70196536 | 2764 | 1:70655375 | 2776 | 1:70889623 | 2777 | 1:70925120 |
| 2811 | 1:71906639 | 2821 | 1:72100009 | 2896 | 1:73841520 | 2929 | 1:74779617 | 2950 | 1:75412538 |
| 2963 | 1:75770166 | 2964 | 1:75770197 | 2965 | 1:75770199 | 2966 | 1:75783854 | 2971 | 1:75829393 |
| 2984 | 1:76114883 | 3005 | 1:76860758 | 3042 | 1:77680697 | 3045 | 1:77701368 | 3070 | 1:78334604 |
| 3071 | 1:78335278 | 3075 | 1:78397021 | 3113 | 1:79354013 | 3224 | 1:81621771 | 3225 | 1:81658300 |
| 3262 | 1:82505239 | 3290 | 1:83265388 | 3301 | 1:83612627 | 3349 | 1:85328309 | 3362 | 1:85790627 |
| 3458 | 1:88779015 | 3466 | 1:88853263 | 3483 | 1:89288724 | 3506 | 1:89791299 | 3511 | 1:89828935 |
| 3523 | 1:90054999 | 3528 | 1:90169751 | 3539 | 1:90383988 | 3572 | 1:91171394 | 3584 | 1:91442961 |
| 3603 | 1:91945075 | 3608 | 1:92172948 | 3613 | 1:92213222 | 3618 | 1:92387065 | 3635 | 1:93023669 |
| 3663 | 1:93808595 | 3690 | 1:94443181 | 3742 | 1:95761493 | 3774 | 1:96387293 | 3818 | 1:97495906 |
| 3831 | 1:97875563 | 3835 | 1:97919421 | 3848 | 1:98319004 | 4084 | 1:105587286 | 4086 | 1:105625485 |
| 4088 | 1:105653161 | 4093 | 1:105729523 | 4163 | 1:107822647 | 4184 | 1:108388088 | 4193 | 1:108564048 |
| 4221 | 1:109261833 | 4235 | 1:109562630 | 4257 | 1:110017436 | 4317 | 1:111454247 | 4321 | 1:111612080 |
| 4346 | 1:112243802 | 4358 | 1:112481363 | 4360 | 1:112532754 | 4385 | 1:112920549 | 4398 | 1:113309692 |
| 4428 | 1:113860732 | 4469 | 1:114867292 | 4471 | 1:114870124 | 4500 | 1:115756884 | 4501 | 1:115772405 |
| 4512 | 1:116396361 | 4572 | 1:117632176 | 4674 | 1:119359964 | 4675 | 1:119363741 | 4707 | 1:120254924 |
| 4715 | 1:120408319 | 4716 | 1:120409192 | 4727 | 1:120576333 | 4737 | 1:120851703 | 4754 | 1:121481221 |
| 4758 | 1:121544917 | 4856 | 1:124230559 | 4872 | 1:124533841 | 4873 | 1:124538558 | 4889 | 1:124936627 |
| 4930 | 1:125655889 | 4933 | 1:125689194 | 4936 | 1:125738236 | 4941 | 1:125837099 | 4945 | 1:125909016 |
| 4952 | 1:126088394 | 5002 | 1:127555264 | 5013 | 1:127861573 | 5035 | 1:128339939 | 5048 | 1:128645880 |
| 5056 | 1:128748369 | 5218 | 1:131754936 | 5233 | 1:132161523 | 5273 | 1:133625347 | 5304 | 1:134472909 |
| 5333 | 1:135062430 | 5365 | 1:135657617 | 5404 | 1:135977700 | 5417 | 1:136372646 | 5458 | 1:137985176 |
| 5493 | 1:139072937 | 5525 | 1:140006988 | 5547 | 1:140601043 | 5559 | 1:140911883 | 5568 | 1:141070658 |
| 5577 | 1:141182724 | 5635 | 1:142058648 | 5639 | 1:142168313 | 5683 | 1:142908923 | 5952 | 1:148034123 |
| 5953 | 1:148040606 | 5954 | 1:148040677 | 5955 | 1:148044952 | 5956 | 1:148046284 | 5957 | 1:148046462 |
| 5958 | 1:148047932 | 5959 | 1:148053077 | 5960 | 1:148055822 | 5961 | 1:148058260 | 5962 | 1:148073345 |
| 5963 | 1:148073444 | 5964 | 1:148075396 | 5974 | 1:148329401 | 5980 | 1:148487864 | 6038 | 1:149409627 |
| 6039 | 1:149411937 | 6040 | 1:149413628 | 6045 | 1:149560052 | 6060 | 1:149819819 | 6139 | 1:151376946 |
| 6140 | 1:151406584 | 6141 | 1:151407059 | 6142 | 1:151407604 | 6152 | 1:151612724 | 6169 | 1:151767739 |
| 6171 | 1:151799413 | 6172 | 1:151799855 | 6173 | 1:151799970 | 6174 | 1:151800242 | 6175 | 1:151801164 |
| 6176 | 1:151801587 | 6177 | 1:151801737 | 6178 | 1:151801752 | 6191 | 1:152091262 | 6201 | 1:152265191 |
| 6202 | 1:152266056 | 6203 | 1:152266869 | 6204 | 1:152267613 | 6205 | 1:152267787 | 6206 | 1:152268300 |
| 6207 | 1:152268458 | 6213 | 1:152339615 | 6214 | 1:152343252 | 6216 | 1:152348515 | 6217 | 1:152349140 |
| 6219 | 1:152350298 | 6220 | 1:152351423 | 6221 | 1:152353474 | 6222 | 1:152353711 | 6223 | 1:152353915 |
| 6224 | 1:152354049 | 6225 | 1:152354217 | 6226 | 1:152354593 | 6227 | 1:152354711 | 6228 | 1:152355277 |
| 6229 | 1:152355296 | 6230 | 1:152355677 | 6231 | 1:152363064 | 6236 | 1:152422417 | 6266 | 1:152974350 |
| 6287 | 1:153459144 | 6356 | 1:154782431 | 6390 | 1:155509473 | 6395 | 1:155617067 | 6397 | 1:155639845 |
| 6398 | 1:155669488 | 6406 | 1:155709356 | 6410 | 1:155739527 | 6415 | 1:155837092 | 6420 | 1:155936300 |
| 6426 | 1:156024716 | 6437 | 1:156119189 | 6463 | 1:156640964 | 6466 | 1:156695097 | 6470 | 1:156760308 |
| 6486 | 1:157055516 | 6504 | 1:157454125 | 6507 | 1:157551305 | 6553 | 2:894974 | 6604 | 2:1577280 |
| 6679 | 2:3866542 | 6686 | 2:3991289 | 6688 | 2:4033904 | 6690 | 2:4040966 | 6691 | 2:4053609 |
| 6707 | 2:4311756 | 6747 | 2:5066365 | 6793 | 2:5931885 | 6795 | 2:5954528 | 6847 | 2:6974699 |
| 6858 | 2:7193712 | 6872 | 2:7370181 | 6879 | 2:7526996 | 6881 | 2:7574742 | 6942 | 2:9325130 |
| 6974 | 2:10332348 | 7018 | 2:11668736 | 7023 | 2:11736825 | 7056 | 2:13064383 | 7074 | 2:13460557 |
| 7080 | 2:13648131 | 7107 | 2:14339353 | 7122 | 2:14701151 | 7166 | 2:15712399 | 7233 | 2:17570676 |
| 7234 | 2:17577794 | 7308 | 2:18634499 | 7318 | 2:18907347 | 7319 | 2:18942177 | 7320 | 2:18943474 |
| 7321 | 2:18951656 | 7322 | 2:18958579 | 7323 | 2:18964610 | 7324 | 2:18967934 | 7333 | 2:19110921 |
| 7382 | 2:20297370 | 7385 | 2:20378241 | 7392 | 2:20658754 | 7396 | 2:20683165 | 7424 | 2:21454390 |
| 7453 | 2:22063850 | 7454 | 2:22086957 | 7457 | 2:22138573 | 7515 | 2:23573584 | 7538 | 2:24013539 |
| 7549 | 2:24246589 | 7565 | 2:24496758 | 7566 | 2:24500311 | 7567 | 2:24524614 | 7570 | 2:24563421 |
| 7588 | 2:24952433 | 7603 | 2:25454747 | 7645 | 2:26254049 | 7656 | 2:26496144 | 7725 | 2:27887225 |
| 7734 | 2:28111908 | 7757 | 2:28481427 | 7809 | 2:29740566 | 7881 | 2:31547829 | 7957 | 2:33826799 |
| 8010 | 2:35264773 | 8019 | 2:35614289 | 8041 | 2:36281226 | 8076 | 2:37317121 | 8084 | 2:37618680 |
| 8086 | 2:37625413 | 8229 | 2:41341923 | 8233 | 2:41443343 | 8234 | 2:41451445 | 8235 | 2:41463995 |
| 8236 | 2:41476600 | 8239 | 2:41525245 | 8240 | 2:41526634 | 8241 | 2:41546403 | 8242 | 2:41555839 |
| 8243 | 2:41555881 | 8244 | 2:41556165 | 8248 | 2:41609088 | 8249 | 2:41648397 | 8263 | 2:42040167 |
| 8264 | 2:42120013 | 8283 | 2:42489642 | 8284 | 2:42493609 | 8285 | 2:42495164 | 8313 | 2:43227689 |
| 8369 | 2:44933199 | 8396 | 2:45615535 | 8398 | 2:45632178 | 8409 | 2:45890270 | 8416 | 2:46041603 |
| 8493 | 2:47767849 | 8507 | 2:48158383 | 8511 | 2:48296454 | 8537 | 2:48966694 | 8542 | 2:48995422 |
| 8546 | 2:49074113 | 8587 | 2:50450300 | 8631 | 2:51841153 | 8632 | 2:51841281 | 8633 | 2:51845196 |
| 8634 | 2:51846388 | 8635 | 2:51848506 | 8636 | 2:51849168 | 8637 | 2:51850817 | 8638 | 2:51851321 |
| 8708 | 2:53799255 | 8721 | 2:54148820 | 8743 | 2:54689808 | 8751 | 2:54852723 | 8752 | 2:54874680 |
| 8786 | 2:55757496 | 8788 | 2:55856268 | 8836 | 2:57348471 | 8850 | 2:57670111 | 8867 | 2:58206752 |
| 8868 | 2:58209897 | 8870 | 2:58220436 | 8871 | 2:58224932 | 8872 | 2:58235017 | 8895 | 2:58894479 |
| 8937 | 2:59886642 | 8939 | 2:59894079 | 8941 | 2:59895422 | 8953 | 2:60262781 | 8986 | 2:61265032 |
| 9027 | 2:62337521 | 9064 | 2:63236556 | 9082 | 2:63698530 | 9103 | 2:64357206 | 9104 | 2:64367151 |
| 9106 | 2:64369812 | 9108 | 2:64437590 | 9109 | 2:64438167 | 9110 | 2:64438313 | 9111 | 2:64438576 |
| 9112 | 2:64438580 | 9113 | 2:64438635 | 9114 | 2:64438908 | 9122 | 2:64709919 | 9123 | 2:64711805 |
| 9144 | 2:65152676 | 9170 | 2:65933901 | 9210 | 2:67245827 | 9238 | 2:68102588 | 9241 | 2:68162816 |
| 9257 | 2:68641959 | 9282 | 2:69654612 | 9286 | 2:69797972 | 9289 | 2:69913137 | 9309 | 2:70434838 |
| 9326 | 2:70835144 | 9351 | 2:71366915 | 9354 | 2:71385196 | 9380 | 2:72368600 | 9431 | 2:73656519 |
| 9432 | 2:73692943 | 9438 | 2:73821323 | 9450 | 2:74104600 | 9460 | 2:74465077 | 9499 | 2:75629903 |
| 9522 | 2:76151639 | 9528 | 2:76318715 | 9572 | 2:77446117 | 9613 | 2:78673595 | 9630 | 2:78917434 |
| 9682 | 2:80074128 | 9733 | 2:82257633 | 9802 | 2:84430538 | 9875 | 2:86138420 | 9884 | 2:86394912 |
| 10120 | 2:91927539 | 10135 | 2:92191854 | 10175 | 2:93130350 | 10210 | 2:93703916 | 10252 | 2:94676418 |
| 10283 | 2:95426880 | 10287 | 2:95450849 | 10300 | 2:95820889 | 10323 | 2:96340062 | 10366 | 2:97323100 |
| 10378 | 2:97726887 | 10382 | 2:97746848 | 10396 | 2:98170549 | 10402 | 2:98356021 | 10418 | 2:98570804 |
| 10421 | 2:98681146 | 10424 | 2:98710984 | 10458 | 2:99816412 | 10462 | 2:99892472 | 10475 | 2:100046086 |
| 10476 | 2:100060098 | 10482 | 2:100180128 | 10493 | 2:100545847 | 10503 | 2:100804647 | 10519 | 2:101319592 |
| 10560 | 2:102476291 | 10585 | 2:103244299 | 10601 | 2:103704057 | 10604 | 2:103752303 | 10606 | 2:103798942 |
| 10619 | 2:104096932 | 10723 | 2:106585047 | 10741 | 2:106795862 | 10757 | 2:107198319 | 10759 | 2:107259324 |
| 10775 | 2:107639270 | 10795 | 2:107803660 | 10816 | 2:108400383 | 10844 | 2:108979070 | 10883 | 2:109843763 |
| 10889 | 2:109962178 | 10898 | 2:110266270 | 10908 | 2:110533827 | 10913 | 2:110782529 | 10921 | 2:111164890 |
| 10941 | 2:111820434 | 10980 | 2:112795307 | 11039 | 2:113925261 | 11047 | 2:114161924 | 11082 | 2:115099463 |
| 11086 | 2:115273767 | 11117 | 2:115778796 | 11118 | 2:115812040 | 11122 | 2:115883386 | 11129 | 2:116006798 |
| 11136 | 2:116182220 | 11144 | 2:116499676 | 11175 | 2:117182401 | 11219 | 2:118492898 | 11223 | 2:118587365 |
| 11228 | 2:118743982 | 11256 | 2:119406723 | 11265 | 2:119542337 | 11296 | 2:120281653 | 11318 | 2:120899441 |
| 11346 | 2:121587712 | 11400 | 2:123193388 | 11465 | 2:125068403 | 11477 | 2:125260143 | 11482 | 2:125405078 |
| 11524 | 2:126369897 | 11535 | 2:126645449 | 11566 | 2:126956287 | 11600 | 2:127060935 | 11611 | 2:127195966 |
| 11616 | 2:127259539 | 11643 | 2:127688853 | 11647 | 2:127843626 | 11660 | 2:128258137 | 11661 | 2:128269460 |
| 11739 | 2:129924952 | 11741 | 2:130000075 | 11795 | 2:131203320 | 11796 | 2:131203333 | 11797 | 2:131206099 |
| 11802 | 2:131272766 | 11848 | 2:132287345 | 11863 | 2:132630938 | 11864 | 2:132635825 | 11865 | 2:132637425 |
| 11922 | 2:133672767 | 11948 | 2:133927517 | 11962 | 2:134187671 | 11976 | 2:134370377 | 12003 | 2:134731903 |
| 12072 | 2:135676512 | 12100 | 3:74989 | 12107 | 3:258342 | 12138 | 3:907384 | 12182 | 3:1660018 |
| 12186 | 3:1722820 | 12267 | 3:3478015 | 12291 | 3:4097674 | 12293 | 3:4100027 | 12301 | 3:4206473 |
| 12342 | 3:4711535 | 12358 | 3:4936089 | 12364 | 3:5121045 | 12365 | 3:5121673 | 12366 | 3:5126160 |
| 12367 | 3:5130659 | 12386 | 3:5499346 | 12429 | 3:6474877 | 12436 | 3:6635006 | 12448 | 3:6773779 |
| 12474 | 3:7163463 | 12482 | 3:7308711 | 12520 | 3:7916080 | 12530 | 3:7997693 | 12589 | 3:9507918 |
| 12593 | 3:9562991 | 12600 | 3:9657041 | 12615 | 3:9990546 | 12621 | 3:10103527 | 12678 | 3:10496754 |
| 12726 | 3:11145282 | 12768 | 3:12417249 | 12776 | 3:12650225 | 12779 | 3:12749123 | 12804 | 3:13251625 |
| 12806 | 3:13370163 | 12807 | 3:13373604 | 12808 | 3:13375404 | 12812 | 3:13573971 | 12833 | 3:13902991 |
| 12866 | 3:14463788 | 12898 | 3:15197212 | 12926 | 3:15692187 | 12941 | 3:15905069 | 12972 | 3:16573676 |
| 12993 | 3:16936423 | 13136 | 3:21101166 | 13141 | 3:21359229 | 13158 | 3:21799171 | 13166 | 3:21978447 |
| 13170 | 3:22029801 | 13230 | 3:23274662 | 13295 | 3:24786196 | 13318 | 3:25281477 | 13329 | 3:25568097 |
| 13394 | 3:26947828 | 13404 | 3:27140090 | 13421 | 3:27417464 | 13426 | 3:27531689 | 13432 | 3:27586695 |
| 13433 | 3:27587568 | 13434 | 3:27588165 | 13435 | 3:27590766 | 13436 | 3:27590979 | 13437 | 3:27591026 |
| 13438 | 3:27591357 | 13439 | 3:27591992 | 13440 | 3:27592060 | 13441 | 3:27592156 | 13442 | 3:27592425 |
| 13443 | 3:27592963 | 13447 | 3:27618230 | 13448 | 3:27623256 | 13449 | 3:27624318 | 13450 | 3:27624378 |
| 13451 | 3:27626096 | 13488 | 3:28582812 | 13489 | 3:28655869 | 13493 | 3:28699418 | 13494 | 3:28701817 |
| 13496 | 3:28737754 | 13497 | 3:28739308 | 13502 | 3:28795698 | 13503 | 3:28847511 | 13512 | 3:29091766 |
| 13517 | 3:29129658 | 13528 | 3:29280162 | 13530 | 3:29285000 | 13543 | 3:29574496 | 13776 | 3:33446479 |
| 13782 | 3:33558867 | 13874 | 3:35055289 | 13935 | 3:36314410 | 13938 | 3:36323470 | 13942 | 3:36436943 |
| 13943 | 3:36535092 | 13955 | 3:36838325 | 13956 | 3:36846776 | 13971 | 3:37286638 | 14025 | 3:39287939 |
| 14031 | 3:39484220 | 14091 | 3:41318467 | 14096 | 3:41450661 | 14106 | 3:41608150 | 14116 | 3:41941631 |
| 14121 | 3:42042149 | 14131 | 3:42370072 | 14173 | 3:43226312 | 14205 | 3:43968451 | 14208 | 3:44136086 |
| 14252 | 3:45371773 | 14253 | 3:45389341 | 14259 | 3:45661559 | 14287 | 3:46609755 | 14319 | 3:47528437 |
| 14325 | 3:47614770 | 14335 | 3:47862102 | 14340 | 3:47901200 | 14346 | 3:48004343 | 14371 | 3:48662558 |
| 14376 | 3:48794169 | 14383 | 3:48908474 | 14388 | 3:48945044 | 14391 | 3:48996518 | 14393 | 3:49005907 |
| 14397 | 3:49066263 | 14400 | 3:49170559 | 14411 | 3:49437426 | 14430 | 3:49640985 | 14431 | 3:49686014 |
| 14432 | 3:49688113 | 14433 | 3:49693943 | 14434 | 3:49694205 | 14435 | 3:49694911 | 14436 | 3:49695537 |
| 14437 | 3:49695765 | 14438 | 3:49695863 | 14439 | 3:49695894 | 14440 | 3:49696034 | 14441 | 3:49697009 |
| 14442 | 3:49697975 | 14443 | 3:49698099 | 14444 | 3:49698127 | 14445 | 3:49698364 | 14446 | 3:49698389 |
| 14447 | 3:49698398 | 14448 | 3:49698433 | 14449 | 3:49698581 | 14450 | 3:49698599 | 14451 | 3:49698610 |
| 14452 | 3:49698697 | 14453 | 3:49698897 | 14454 | 3:49698954 | 14455 | 3:49698961 | 14456 | 3:49699145 |
| 14457 | 3:49699438 | 14458 | 3:49699942 | 14459 | 3:49700029 | 14460 | 3:49700320 | 14461 | 3:49700692 |
| 14462 | 3:49700695 | 14463 | 3:49700919 | 14464 | 3:49701129 | 14465 | 3:49701149 | 14466 | 3:49701225 |
| 14467 | 3:49701410 | 14468 | 3:49701470 | 14469 | 3:49702278 | 14470 | 3:49702301 | 14476 | 3:49768343 |
| 14489 | 3:50198427 | 14509 | 3:50849745 | 14526 | 3:51610373 | 14542 | 3:51914443 | 14546 | 3:51963753 |
| 14547 | 3:52005921 | 14550 | 3:52127559 | 14559 | 3:52354237 | 14560 | 3:52412518 | 14562 | 3:52416849 |
| 14563 | 3:52423182 | 14564 | 3:52427169 | 14566 | 3:52429848 | 14567 | 3:52433914 | 14570 | 3:52487075 |
| 14577 | 3:52690753 | 14580 | 3:52745287 | 14583 | 3:52852927 | 14591 | 3:53113743 | 14592 | 3:53117054 |
| 14593 | 3:53126092 | 14594 | 3:53126314 | 14595 | 3:53126332 | 14597 | 3:53143352 | 14600 | 3:53244194 |
| 14608 | 3:53474847 | 14610 | 3:53481481 | 14611 | 3:53510393 | 14652 | 3:55080090 | 14670 | 3:55424410 |
| 14740 | 3:57665967 | 14760 | 3:58213744 | 14761 | 3:58216986 | 14771 | 3:58501632 | 14789 | 3:58991070 |
| 14855 | 3:60699483 | 14856 | 3:60699729 | 14857 | 3:60704807 | 14871 | 3:60976891 | 14874 | 3:61002539 |
| 14876 | 3:61050627 | 14911 | 3:62289881 | 14936 | 3:62942816 | 14979 | 3:64119969 | 14994 | 3:64762588 |
| 15003 | 3:65001585 | 15011 | 3:65190175 | 15045 | 3:66062653 | 15056 | 3:66307909 | 15064 | 3:66462222 |
| 15108 | 3:67467438 | 15119 | 3:67681424 | 15160 | 3:68627230 | 15190 | 3:69504184 | 15196 | 3:69782248 |
| 15197 | 3:69782256 | 15198 | 3:69782262 | 15199 | 3:69786350 | 15215 | 3:70251128 | 15260 | 3:71848961 |
| 15262 | 3:71934380 | 15307 | 3:73666374 | 15325 | 3:74232466 | 15331 | 3:74366602 | 15394 | 3:76166365 |
| 15408 | 3:76630621 | 15458 | 3:78060366 | 15471 | 3:78544662 | 15539 | 3:80535834 | 15585 | 3:81521406 |
| 15586 | 3:81525295 | 15639 | 3:82930218 | 15642 | 3:83051313 | 15674 | 3:84083709 | 15679 | 3:84152801 |
| 15685 | 3:84286878 | 15713 | 3:84688663 | 15738 | 3:84955518 | 15745 | 3:85050052 | 15746 | 3:85066223 |
| 15766 | 3:85749188 | 15769 | 3:85769939 | 15774 | 3:85846088 | 15776 | 3:85897724 | 15784 | 3:85979828 |
| 15785 | 3:86001057 | 15786 | 3:86027116 | 15846 | 3:87314340 | 15854 | 3:87568457 | 15855 | 3:87570687 |
| 15857 | 3:87576354 | 15858 | 3:87576887 | 15859 | 3:87596560 | 15865 | 3:87888225 | 15900 | 3:88530363 |
| 15903 | 3:88603888 | 15905 | 3:88620871 | 15926 | 3:88972091 | 15934 | 3:89181812 | 15938 | 3:89279883 |
| 15955 | 3:89599405 | 15989 | 3:90306732 | 15990 | 3:90333561 | 16033 | 3:91219484 | 16036 | 3:91278796 |
| 16051 | 3:91695764 | 16084 | 3:92543467 | 16101 | 3:92905285 | 16113 | 3:93070586 | 16148 | 3:93889901 |
| 16213 | 3:95744017 | 16218 | 3:95893190 | 16222 | 3:96032684 | 16228 | 3:96151926 | 16281 | 3:97566938 |
| 16299 | 3:97901912 | 16346 | 3:99237470 | 16360 | 3:99532212 | 16362 | 3:99579446 | 16424 | 3:100686242 |
| 16429 | 3:100725968 | 16463 | 3:101430182 | 16476 | 3:101718735 | 16488 | 3:102039758 | 16507 | 3:102478118 |
| 16508 | 3:102487038 | 16525 | 3:102814655 | 16545 | 3:103051617 | 16547 | 3:103073339 | 16685 | 3:105603357 |
| 16698 | 3:105784125 | 16760 | 3:106768484 | 16763 | 3:106821154 | 16796 | 3:107517305 | 16797 | 3:107520457 |
| 16805 | 3:107754888 | 16842 | 3:108517017 | 16865 | 3:108844131 | 16904 | 3:109724431 | 16905 | 3:109725745 |
| 16906 | 3:109730150 | 16907 | 3:109731953 | 16908 | 3:109737608 | 16909 | 3:109737977 | 16910 | 3:109738917 |
| 16980 | 3:111463594 | 16984 | 3:111515807 | 17006 | 3:111954834 | 17039 | 3:112612031 | 17040 | 3:112625613 |
| 17055 | 3:112990876 | 17088 | 3:113561927 | 17123 | 3:114126601 | 17131 | 3:114256669 | 17145 | 3:114503215 |
| 17147 | 3:114519254 | 17151 | 3:114571821 | 17153 | 3:114600016 | 17154 | 3:114600017 | 17161 | 3:114722650 |
| 17203 | 3:115249589 | 17205 | 3:115271507 | 17250 | 3:116030538 | 17298 | 3:116861627 | 17300 | 3:116869634 |
| 17301 | 3:116871170 | 17303 | 3:116878333 | 17305 | 3:116901187 | 17306 | 3:116901527 | 17307 | 3:116903799 |
| 17311 | 3:116932009 | 17340 | 3:117275171 | 17356 | 3:117642247 | 17357 | 3:117642545 | 17358 | 3:117643811 |
| 17359 | 3:117644307 | 17398 | 3:118174177 | 17427 | 3:118632548 | 17445 | 3:118859735 | 17446 | 3:118864123 |
| 17451 | 3:118890670 | 17500 | 3:120280786 | 17594 | 4:2379436 | 17634 | 4:3851682 | 17635 | 4:3859753 |
| 17638 | 4:3982830 | 17645 | 4:4155060 | 17665 | 4:4566566 | 17667 | 4:4584480 | 17668 | 4:4589135 |
| 17669 | 4:4591392 | 17670 | 4:4591448 | 17677 | 4:4684194 | 17681 | 4:4780212 | 17682 | 4:4780800 |
| 17683 | 4:4786841 | 17687 | 4:4824857 | 17690 | 4:4865949 | 17694 | 4:4969955 | 17719 | 4:5368040 |
| 17727 | 4:5444338 | 17738 | 4:5704758 | 17765 | 4:6383685 | 17791 | 4:7064916 | 17803 | 4:7319609 |
| 17805 | 4:7325326 | 17815 | 4:7454936 | 17830 | 4:7796044 | 17846 | 4:8193002 | 17869 | 4:8790081 |
| 17881 | 4:9092344 | 17895 | 4:9372959 | 17903 | 4:9479800 | 17917 | 4:9747477 | 17933 | 4:9983918 |
| 17934 | 4:9983950 | 17935 | 4:9984121 | 17936 | 4:9984185 | 17937 | 4:9984248 | 17938 | 4:9984598 |
| 17940 | 4:9986515 | 17971 | 4:10645340 | 17974 | 4:10692378 | 17979 | 4:10807167 | 17980 | 4:10817042 |
| 17982 | 4:10817118 | 18028 | 4:11882269 | 18081 | 4:13277909 | 18083 | 4:13314057 | 18084 | 4:13315343 |
| 18132 | 4:14870332 | 18185 | 4:16314407 | 18193 | 4:16510166 | 18212 | 4:17151245 | 18220 | 4:17227279 |
| 18227 | 4:17429209 | 18244 | 4:17664281 | 18251 | 4:17934274 | 18292 | 4:18879751 | 18295 | 4:18927680 |
| 18301 | 4:18998283 | 18302 | 4:18999825 | 18303 | 4:19004726 | 18304 | 4:19005718 | 18305 | 4:19006333 |
| 18306 | 4:19007854 | 18307 | 4:19007876 | 18308 | 4:19008526 | 18309 | 4:19008760 | 18310 | 4:19010468 |
| 18331 | 4:19684498 | 18332 | 4:19687001 | 18333 | 4:19687703 | 18334 | 4:19688039 | 18335 | 4:19690469 |
| 18336 | 4:19690711 | 18337 | 4:19692144 | 18338 | 4:19692593 | 18339 | 4:19692676 | 18340 | 4:19693098 |
| 18341 | 4:19693119 | 18342 | 4:19694330 | 18343 | 4:19694937 | 18344 | 4:19695576 | 18345 | 4:19695681 |
| 18346 | 4:19696720 | 18347 | 4:19696767 | 18348 | 4:19697089 | 18349 | 4:19697301 | 18350 | 4:19697430 |
| 18351 | 4:19697544 | 18352 | 4:19697663 | 18354 | 4:19748494 | 18359 | 4:19938692 | 18361 | 4:19989444 |
| 18375 | 4:20261489 | 18376 | 4:20265073 | 18392 | 4:20703476 | 18401 | 4:20822864 | 18421 | 4:21167294 |
| 18431 | 4:21342092 | 18432 | 4:21358199 | 18440 | 4:21699344 | 18457 | 4:22138635 | 18466 | 4:22471078 |
| 18468 | 4:22523960 | 18481 | 4:22728193 | 18490 | 4:22923982 | 18497 | 4:23121119 | 18500 | 4:23196768 |
| 18504 | 4:23389777 | 18530 | 4:24084837 | 18546 | 4:24614792 | 18563 | 4:25092475 | 18600 | 4:26062712 |
| 18625 | 4:26743642 | 18628 | 4:26796377 | 18632 | 4:26851816 | 18643 | 4:27158677 | 18656 | 4:27519461 |
| 18679 | 4:28114813 | 18697 | 4:28590705 | 18717 | 4:29227466 | 18733 | 4:29791373 | 18759 | 4:30557284 |
| 18768 | 4:30768290 | 18782 | 4:31109031 | 18806 | 4:31790626 | 18808 | 4:31802602 | 18827 | 4:32352959 |
| 18838 | 4:32687897 | 18870 | 4:33603343 | 18902 | 4:34379775 | 18952 | 4:36053918 | 18953 | 4:36057890 |
| 18966 | 4:36473548 | 18993 | 4:37223623 | 19001 | 4:37381316 | 19019 | 4:38054717 | 19024 | 4:38091098 |
| 19030 | 4:38294871 | 19041 | 4:38579078 | 19042 | 4:38591331 | 19060 | 4:39231026 | 19066 | 4:39400801 |
| 19084 | 4:39970721 | 19114 | 4:40595576 | 19133 | 4:40929985 | 19178 | 4:42197758 | 19207 | 4:43094045 |
| 19209 | 4:43138220 | 19236 | 4:43793806 | 19294 | 4:45527845 | 19336 | 4:46745755 | 19366 | 4:47300099 |
| 19380 | 4:47488474 | 19388 | 4:47696820 | 19425 | 4:49010238 | 19454 | 4:49228470 | 19465 | 4:49442617 |
| 19505 | 4:50866615 | 19529 | 4:51642850 | 19540 | 4:52014153 | 19574 | 4:53063969 | 19623 | 4:54533598 |
| 19654 | 4:55652059 | 19655 | 4:55666248 | 19666 | 4:55979945 | 19678 | 4:56152616 | 19746 | 4:58369087 |
| 19753 | 4:58591231 | 19754 | 4:58596005 | 19769 | 4:59119437 | 19775 | 4:59214261 | 19791 | 4:59626845 |
| 19794 | 4:59754417 | 19815 | 4:60395032 | 19869 | 4:61855772 | 19915 | 4:63120245 | 19920 | 4:63231993 |
| 19939 | 4:63706634 | 19941 | 4:63737742 | 19965 | 4:64527362 | 19979 | 4:64927929 | 19981 | 4:64973879 |
| 20073 | 4:67329757 | 20080 | 4:67492078 | 20083 | 4:67548081 | 20121 | 4:68635250 | 20122 | 4:68672213 |
| 20195 | 4:70609606 | 20233 | 4:71767720 | 20235 | 4:71789741 | 20358 | 4:75159207 | 20362 | 4:75242210 |
| 20385 | 4:75797380 | 20413 | 4:76318250 | 20436 | 4:77029452 | 20481 | 4:77948146 | 20491 | 4:78185575 |
| 20539 | 4:79657303 | 20543 | 4:79839208 | 20568 | 4:80186925 | 20573 | 4:80245076 | 20587 | 4:80584868 |
| 20592 | 4:80719912 | 20661 | 4:82586451 | 20666 | 4:82698761 | 20712 | 4:83936797 | 20729 | 4:84451586 |
| 20765 | 4:85525303 | 20774 | 4:85682095 | 20781 | 4:85880701 | 20796 | 4:86159050 | 20846 | 4:87474051 |
| 20850 | 4:87586277 | 20873 | 4:88094522 | 20894 | 4:88685340 | 20914 | 4:89467898 | 20924 | 4:89712815 |
| 20974 | 4:90944945 | 21037 | 4:92282682 | 21054 | 4:92473823 | 21057 | 4:92523126 | 21063 | 4:92640436 |
| 21103 | 4:93420321 | 21105 | 4:93454704 | 21113 | 4:93574942 | 21118 | 4:93658997 | 21176 | 4:95336071 |
| 21177 | 4:95348378 | 21181 | 4:95493235 | 21227 | 4:96019036 | 21262 | 4:96809210 | 21279 | 4:96825379 |
| 21360 | 4:96942694 | 21366 | 4:97025110 | 21436 | 4:97663817 | 21456 | 4:98220002 | 21526 | 4:100120727 |
| 21537 | 4:100380798 | 21545 | 4:100648109 | 21550 | 4:100737518 | 21555 | 4:100801331 | 21644 | 4:102776227 |
| 21654 | 4:103076079 | 21677 | 4:103575595 | 21680 | 4:103670470 | 21746 | 4:105364910 | 21757 | 4:105516749 |
| 21761 | 4:105568960 | 21777 | 4:106062418 | 21819 | 4:106715919 | 21888 | 4:107648399 | 21906 | 4:108309499 |
| 21907 | 4:108319723 | 21926 | 4:108947713 | 21940 | 4:109363070 | 21947 | 4:109525615 | 21948 | 4:109528067 |
| 21949 | 4:109528137 | 21950 | 4:109529095 | 21951 | 4:109529531 | 21952 | 4:109529976 | 21953 | 4:109530257 |
| 21954 | 4:109530338 | 21955 | 4:109532512 | 21956 | 4:109533036 | 21957 | 4:109538711 | 21958 | 4:109538973 |
| 21959 | 4:109545654 | 21961 | 4:109576187 | 21985 | 4:110235765 | 21986 | 4:110257114 | 21999 | 4:110637707 |
| 22000 | 4:110672104 | 22001 | 4:110703849 | 22014 | 4:110890327 | 22025 | 4:111120368 | 22026 | 4:111120557 |
| 22061 | 4:111707261 | 22062 | 4:111708263 | 22063 | 4:111711546 | 22064 | 4:111711848 | 22065 | 4:111713444 |
| 22086 | 4:112129918 | 22089 | 4:112149705 | 22095 | 4:112210325 | 22107 | 4:112369141 | 22119 | 4:112555531 |
| 22179 | 4:113489475 | 22192 | 4:113598124 | 22193 | 4:113600602 | 22194 | 4:113601769 | 22196 | 4:113610307 |
| 22197 | 4:113612170 | 22198 | 4:113614202 | 22202 | 4:113636177 | 22203 | 4:113637533 | 22204 | 4:113639510 |
| 22205 | 4:113645913 | 22206 | 4:113646772 | 22207 | 4:113647228 | 22208 | 4:113647637 | 22209 | 4:113649086 |
| 22210 | 4:113650751 | 22211 | 4:113653050 | 22212 | 4:113670640 | 22213 | 4:113670882 | 22214 | 4:113671866 |
| 22215 | 4:113673914 | 22216 | 4:113673955 | 22217 | 4:113676621 | 22218 | 4:113680315 | 22351 | 4:115923041 |
| 22368 | 4:116278293 | 22377 | 4:116438965 | 22393 | 4:116746277 | 22401 | 4:116897937 | 22404 | 4:116926240 |
| 22405 | 4:116961074 | 22514 | 4:119167733 | 22520 | 4:119293731 | 22547 | 5:35727 | 22548 | 5:42965 |
| 22549 | 5:46543 | 22550 | 5:56066 | 22551 | 5:57672 | 22554 | 5:90000 | 22555 | 5:93744 |
| 22556 | 5:95203 | 22557 | 5:102783 | 22558 | 5:126748 | 22559 | 5:149452 | 22560 | 5:156011 |
| 22562 | 5:157715 | 22563 | 5:159858 | 22578 | 5:818560 | 22609 | 5:1354890 | 22616 | 5:1388630 |
| 22619 | 5:1458836 | 22642 | 5:1946239 | 22703 | 5:3492443 | 22727 | 5:4322265 | 22743 | 5:4570191 |
| 22752 | 5:4731333 | 22754 | 5:4732613 | 22775 | 5:5220278 | 22799 | 5:5717840 | 22851 | 5:6927428 |
| 22853 | 5:6949366 | 22892 | 5:7781205 | 22896 | 5:7895175 | 22903 | 5:8176883 | 22949 | 5:9380271 |
| 22955 | 5:9551052 | 22956 | 5:9555919 | 22960 | 5:9636042 | 22996 | 5:10313930 | 23013 | 5:10668196 |
| 23026 | 5:10924954 | 23029 | 5:10994036 | 23065 | 5:12039811 | 23100 | 5:12501985 | 23102 | 5:12524034 |
| 23125 | 5:13140668 | 23167 | 5:14461980 | 23181 | 5:14793206 | 23214 | 5:15447067 | 23218 | 5:15558924 |
| 23257 | 5:16336560 | 23268 | 5:16719670 | 23269 | 5:16739498 | 23279 | 5:17077362 | 23280 | 5:17078096 |
| 23292 | 5:17322229 | 23296 | 5:17492634 | 23298 | 5:17500602 | 23301 | 5:17665160 | 23307 | 5:17903579 |
| 23313 | 5:18155232 | 23326 | 5:18518099 | 23364 | 5:19695581 | 23400 | 5:20738200 | 23430 | 5:21391973 |
| 23432 | 5:21432976 | 23439 | 5:21643782 | 23440 | 5:21648427 | 23451 | 5:21871823 | 23459 | 5:21991889 |
| 23484 | 5:22479526 | 23485 | 5:22487130 | 23543 | 5:23622599 | 23555 | 5:23928759 | 23558 | 5:23956744 |
| 23613 | 5:25183734 | 23665 | 5:26532385 | 23677 | 5:26846005 | 23680 | 5:26972277 | 23681 | 5:26972772 |
| 23690 | 5:27127258 | 23702 | 5:27439061 | 23707 | 5:27544684 | 23733 | 5:27949133 | 23757 | 5:28498202 |
| 23799 | 5:29557818 | 23812 | 5:29809074 | 23822 | 5:29994317 | 23825 | 5:30019258 | 23946 | 5:33177185 |
| 23952 | 5:33331535 | 23953 | 5:33336405 | 23954 | 5:33337110 | 23968 | 5:33738237 | 24007 | 5:34880473 |
| 24042 | 5:35923865 | 24045 | 5:36012922 | 24048 | 5:36038265 | 24049 | 5:36042606 | 24058 | 5:36409810 |
| 24074 | 5:36939207 | 24120 | 5:37996697 | 24182 | 5:39657770 | 24190 | 5:39821210 | 24193 | 5:39927222 |
| 24238 | 5:41211155 | 24240 | 5:41232876 | 24241 | 5:41235105 | 24242 | 5:41236186 | 24258 | 5:41776747 |
| 24279 | 5:42522952 | 24322 | 5:43563557 | 24327 | 5:43634786 | 24366 | 5:44572552 | 24385 | 5:45181510 |
| 24386 | 5:45181566 | 24402 | 5:45615706 | 24417 | 5:45973787 | 24431 | 5:46166472 | 24441 | 5:46464570 |
| 24443 | 5:46517477 | 24452 | 5:46782312 | 24458 | 5:46936034 | 24459 | 5:46938241 | 24460 | 5:46938449 |
| 24461 | 5:46940754 | 24462 | 5:46941677 | 24475 | 5:47204347 | 24522 | 5:48389782 | 24529 | 5:48667259 |
| 24535 | 5:48760782 | 24540 | 5:48866791 | 24550 | 5:49202198 | 24559 | 5:49562957 | 24562 | 5:49625601 |
| 24570 | 5:49842789 | 24607 | 5:50836826 | 24779 | 5:55323383 | 24806 | 5:55945914 | 24858 | 5:57094271 |
| 24893 | 5:58015125 | 24920 | 5:59115316 | 24926 | 5:59337375 | 24942 | 5:59884753 | 24948 | 5:60008612 |
| 24991 | 5:60879895 | 24994 | 5:60984488 | 24999 | 5:61164840 | 25018 | 5:61884469 | 25034 | 5:62444037 |
| 25035 | 5:62445409 | 25036 | 5:62449226 | 25037 | 5:62449848 | 25038 | 5:62450932 | 25055 | 5:63033268 |
| 25082 | 5:63536637 | 25111 | 5:64402956 | 25124 | 5:64829651 | 25128 | 5:64926399 | 25129 | 5:64926523 |
| 25130 | 5:64928264 | 25164 | 5:65659261 | 25171 | 5:65755099 | 25197 | 5:66595784 | 25208 | 5:66799811 |
| 25219 | 5:67271370 | 25335 | 5:69618545 | 25426 | 5:71726794 | 25464 | 5:72378602 | 25466 | 5:72402551 |
| 25482 | 5:73010927 | 25486 | 5:73094311 | 25488 | 5:73189660 | 25550 | 5:74746217 | 25597 | 5:75667991 |
| 25606 | 5:75883986 | 25611 | 5:75946414 | 25665 | 5:77347106 | 25680 | 5:77945857 | 25725 | 5:78973946 |
| 25727 | 5:79017627 | 25735 | 5:79334960 | 25748 | 5:79714596 | 25749 | 5:79722250 | 25781 | 5:80877376 |
| 25791 | 5:81278052 | 25871 | 5:82792399 | 25884 | 5:83174887 | 25891 | 5:83348374 | 25900 | 5:83570417 |
| 25915 | 5:83863121 | 25918 | 5:83916047 | 25923 | 5:84010266 | 25926 | 5:84064602 | 25927 | 5:84064623 |
| 25928 | 5:84064772 | 25929 | 5:84065038 | 25930 | 5:84065171 | 25931 | 5:84065332 | 25932 | 5:84065339 |
| 25933 | 5:84065355 | 25934 | 5:84066088 | 25935 | 5:84066287 | 25936 | 5:84067645 | 25937 | 5:84068297 |
| 25938 | 5:84068606 | 25939 | 5:84068687 | 25940 | 5:84068732 | 25941 | 5:84068756 | 25942 | 5:84070079 |
| 25943 | 5:84070128 | 25944 | 5:84076319 | 25945 | 5:84076527 | 25946 | 5:84077100 | 25947 | 5:84080168 |
| 25949 | 5:84086306 | 25950 | 5:84086861 | 25951 | 5:84090372 | 25952 | 5:84090487 | 25953 | 5:84097284 |
| 25954 | 5:84097885 | 25955 | 5:84097985 | 25956 | 5:84099214 | 25957 | 5:84100319 | 25958 | 5:84101337 |
| 25959 | 5:84101438 | 25960 | 5:84101462 | 25961 | 5:84101494 | 25962 | 5:84101519 | 25963 | 5:84101555 |
| 25964 | 5:84101619 | 25965 | 5:84101701 | 25966 | 5:84102206 | 25967 | 5:84102236 | 25969 | 5:84103792 |
| 25970 | 5:84103930 | 25971 | 5:84104200 | 25972 | 5:84107013 | 25974 | 5:84115513 | 25975 | 5:84115550 |
| 25976 | 5:84115605 | 25977 | 5:84115682 | 25978 | 5:84115698 | 25979 | 5:84115763 | 25980 | 5:84115908 |
| 25981 | 5:84116028 | 25982 | 5:84116506 | 25983 | 5:84116583 | 25984 | 5:84119378 | 25985 | 5:84119408 |
| 25986 | 5:84119435 | 25987 | 5:84120471 | 25988 | 5:84120477 | 25989 | 5:84120540 | 25990 | 5:84120562 |
| 25991 | 5:84120612 | 25992 | 5:84120761 | 25993 | 5:84120772 | 25994 | 5:84120800 | 25995 | 5:84120828 |
| 25996 | 5:84120900 | 25997 | 5:84120980 | 25998 | 5:84120987 | 25999 | 5:84121087 | 26000 | 5:84121318 |
| 26001 | 5:84122651 | 26002 | 5:84122709 | 26003 | 5:84122905 | 26004 | 5:84123385 | 26005 | 5:84123428 |
| 26006 | 5:84124508 | 26007 | 5:84124539 | 26008 | 5:84124557 | 26009 | 5:84124558 | 26010 | 5:84124682 |
| 26011 | 5:84125648 | 26012 | 5:84125855 | 26013 | 5:84126181 | 26014 | 5:84126183 | 26015 | 5:84126222 |
| 26016 | 5:84126766 | 26017 | 5:84126941 | 26018 | 5:84126965 | 26019 | 5:84126986 | 26020 | 5:84127998 |
| 26021 | 5:84128048 | 26022 | 5:84128850 | 26023 | 5:84128968 | 26024 | 5:84129307 | 26025 | 5:84129349 |
| 26026 | 5:84129709 | 26027 | 5:84130342 | 26028 | 5:84130419 | 26029 | 5:84130836 | 26030 | 5:84130988 |
| 26031 | 5:84131404 | 26032 | 5:84131699 | 26033 | 5:84131703 | 26034 | 5:84131742 | 26035 | 5:84132713 |
| 26036 | 5:84132801 | 26037 | 5:84132954 | 26038 | 5:84133129 | 26039 | 5:84133144 | 26040 | 5:84133170 |
| 26041 | 5:84133302 | 26042 | 5:84133384 | 26043 | 5:84133400 | 26044 | 5:84133418 | 26045 | 5:84135047 |
| 26046 | 5:84135179 | 26047 | 5:84135274 | 26048 | 5:84137923 | 26049 | 5:84138207 | 26050 | 5:84138277 |
| 26051 | 5:84138403 | 26052 | 5:84138617 | 26053 | 5:84138641 | 26054 | 5:84138927 | 26055 | 5:84142947 |
| 26056 | 5:84143312 | 26069 | 5:84405330 | 26105 | 5:85329574 | 26123 | 5:85783695 | 26129 | 5:85892084 |
| 26147 | 5:86451423 | 26153 | 5:86534299 | 26158 | 5:86590201 | 26161 | 5:86629956 | 26175 | 5:86950590 |
| 26207 | 5:87624956 | 26209 | 5:87645403 | 26219 | 5:87859562 | 26287 | 5:88745049 | 26319 | 5:89399078 |
| 26320 | 5:89427836 | 26334 | 5:89821006 | 26385 | 5:91059021 | 26400 | 5:91433996 | 26401 | 5:91479219 |
| 26434 | 5:92344711 | 26436 | 5:92351975 | 26437 | 5:92360410 | 26440 | 5:92389962 | 26441 | 5:92399584 |
| 26442 | 5:92402496 | 26443 | 5:92402509 | 26444 | 5:92407767 | 26445 | 5:92412423 | 26446 | 5:92412426 |
| 26447 | 5:92412918 | 26448 | 5:92415826 | 26449 | 5:92416355 | 26450 | 5:92418513 | 26451 | 5:92422512 |
| 26452 | 5:92422802 | 26453 | 5:92423893 | 26454 | 5:92426805 | 26455 | 5:92428903 | 26456 | 5:92428912 |
| 26457 | 5:92429850 | 26458 | 5:92429875 | 26459 | 5:92430956 | 26460 | 5:92431470 | 26461 | 5:92431717 |
| 26462 | 5:92431719 | 26463 | 5:92432015 | 26464 | 5:92432232 | 26465 | 5:92432425 | 26466 | 5:92432633 |
| 26472 | 5:92541180 | 26487 | 5:92897747 | 26582 | 5:95173664 | 26584 | 5:95197902 | 26656 | 5:96676782 |
| 26698 | 5:97374091 | 26709 | 5:97622463 | 26728 | 5:98063850 | 26729 | 5:98064276 | 26735 | 5:98193113 |
| 26736 | 5:98202197 | 26738 | 5:98236533 | 26768 | 5:98748721 | 26772 | 5:98826571 | 26828 | 5:99948490 |
| 26861 | 5:100535488 | 26863 | 5:100631069 | 26870 | 5:100703214 | 26884 | 5:100953490 | 26893 | 5:101083268 |
| 26894 | 5:101089292 | 26899 | 5:101221558 | 26914 | 5:101607756 | 26921 | 5:101673726 | 26928 | 5:101914630 |
| 26940 | 5:102460767 | 26943 | 5:102476964 | 26968 | 5:103154349 | 26986 | 5:103552417 | 27001 | 5:103882706 |
| 27005 | 5:103981305 | 27068 | 5:105100005 | 27114 | 5:105867389 | 27140 | 5:106221085 | 27148 | 5:106293076 |
| 27162 | 5:106480592 | 27188 | 5:106887255 | 27202 | 5:107008747 | 27222 | 5:107342588 | 27232 | 5:107495491 |
| 27234 | 5:107522927 | 27269 | 5:108224683 | 27274 | 5:108258258 | 27280 | 5:108472906 | 27281 | 5:108473260 |
| 27282 | 5:108476147 | 27283 | 5:108478132 | 27284 | 5:108485933 | 27285 | 5:108486228 | 27286 | 5:108486756 |
| 27287 | 5:108487832 | 27288 | 5:108489677 | 27289 | 5:108490693 | 27290 | 5:108490734 | 27295 | 5:108546887 |
| 27297 | 5:108575310 | 27399 | 5:110511118 | 27440 | 5:111144555 | 27462 | 5:111761777 | 27467 | 5:111860816 |
| 27481 | 5:112115946 | 27519 | 5:112982896 | 27525 | 5:113133130 | 27539 | 5:113320727 | 27547 | 5:113650431 |
| 27566 | 5:114143008 | 27569 | 5:114206160 | 27572 | 5:114283857 | 27580 | 5:114719162 | 27583 | 5:114775926 |
| 27605 | 5:115427295 | 27652 | 5:116126971 | 27656 | 5:116179833 | 27676 | 5:116586289 | 27677 | 5:116588965 |
| 27678 | 5:116590488 | 27679 | 5:116590506 | 27680 | 5:116590745 | 27682 | 5:116592015 | 27683 | 5:116592619 |
| 27684 | 5:116592903 | 27687 | 5:116593192 | 27688 | 5:116593569 | 27694 | 5:116594398 | 27695 | 5:116594541 |
| 27696 | 5:116594546 | 27697 | 5:116594548 | 27698 | 5:116594847 | 27699 | 5:116595244 | 27700 | 5:116595727 |
| 27701 | 5:116595799 | 27702 | 5:116596186 | 27704 | 5:116596441 | 27705 | 5:116596625 | 27706 | 5:116596745 |
| 27707 | 5:116596913 | 27708 | 5:116597118 | 27711 | 5:116598702 | 27712 | 5:116598835 | 27715 | 5:116602527 |
| 27721 | 5:116677042 | 27731 | 5:116722878 | 27808 | 5:117709009 | 27809 | 5:117711361 | 27813 | 5:117773986 |
| 27826 | 5:117998759 | 27831 | 5:118098871 | 27838 | 5:118250217 | 27860 | 5:118703768 | 27866 | 5:118745750 |
| 27903 | 5:119271970 | 27908 | 5:119361103 | 27916 | 5:119587043 | 27948 | 6:528320 | 27970 | 6:993117 |
| 27999 | 6:1950764 | 28000 | 6:1951302 | 28001 | 6:1951498 | 28002 | 6:1951714 | 28003 | 6:1951719 |
| 28004 | 6:1951800 | 28005 | 6:1951981 | 28006 | 6:1952392 | 28007 | 6:1955233 | 28008 | 6:1955330 |
| 28009 | 6:1955401 | 28010 | 6:1955549 | 28011 | 6:1955698 | 28012 | 6:1956232 | 28013 | 6:1956278 |
| 28014 | 6:1957428 | 28015 | 6:1957620 | 28016 | 6:1957800 | 28017 | 6:1957812 | 28018 | 6:1957895 |
| 28019 | 6:1958152 | 28020 | 6:1962365 | 28021 | 6:1963113 | 28022 | 6:1963759 | 28023 | 6:1963825 |
| 28024 | 6:1963953 | 28026 | 6:1973036 | 28027 | 6:1974486 | 28028 | 6:1974895 | 28061 | 6:2801468 |
| 28063 | 6:2860306 | 28064 | 6:2862765 | 28072 | 6:3067202 | 28074 | 6:3073689 | 28075 | 6:3084367 |
| 28129 | 6:3786375 | 28155 | 6:4418616 | 28168 | 6:4782253 | 28170 | 6:4824255 | 28221 | 6:6133626 |
| 28238 | 6:6309983 | 28278 | 6:7061434 | 28286 | 6:7250536 | 28293 | 6:7394514 | 28319 | 6:8317563 |
| 28336 | 6:8929534 | 28337 | 6:8944681 | 28339 | 6:8955494 | 28341 | 6:8960665 | 28343 | 6:8972751 |
| 28345 | 6:8980990 | 28346 | 6:8985187 | 28347 | 6:8989449 | 28352 | 6:9079158 | 28353 | 6:9081859 |
| 28354 | 6:9085203 | 28356 | 6:9094449 | 28373 | 6:9291690 | 28377 | 6:9465992 | 28389 | 6:9964731 |
| 28390 | 6:9965161 | 28391 | 6:9976901 | 28393 | 6:9992803 | 28395 | 6:10029462 | 28409 | 6:10453731 |
| 28422 | 6:10849905 | 28435 | 6:11149738 | 28440 | 6:11315175 | 28605 | 6:15051875 | 28641 | 6:15680456 |
| 28686 | 6:16603866 | 28718 | 6:17241712 | 28757 | 6:18035615 | 28758 | 6:18035662 | 28759 | 6:18035884 |
| 28760 | 6:18036845 | 28766 | 6:18057767 | 28795 | 6:18806853 | 28819 | 6:19264271 | 28820 | 6:19264527 |
| 28852 | 6:19965110 | 28878 | 6:20517010 | 28888 | 6:20729903 | 28944 | 6:22097118 | 28970 | 6:22554116 |
| 29044 | 6:24253816 | 29057 | 6:24617112 | 29061 | 6:24750491 | 29069 | 6:24940966 | 29114 | 6:25994106 |
| 29115 | 6:25995000 | 29123 | 6:26081649 | 29151 | 6:26895539 | 29159 | 6:27119474 | 29179 | 6:27632577 |
| 29182 | 6:27765059 | 29183 | 6:27794604 | 29196 | 6:28156905 | 29204 | 6:28392137 | 29233 | 6:29155222 |
| 29374 | 6:30901671 | 29490 | 6:34238062 | 29722 | 6:34572571 | 29939 | 6:35180069 | 29996 | 6:35924006 |
| 29997 | 6:35924204 | 29998 | 6:35925489 | 29999 | 6:35931470 | 30000 | 6:35932579 | 30010 | 6:35978601 |
| 30133 | 6:36426239 | 30136 | 6:36429755 | 30138 | 6:36430937 | 30160 | 6:36467397 | 30164 | 6:36475104 |
| 30165 | 6:36477167 | 30168 | 6:36481433 | 30169 | 6:36481434 | 30171 | 6:36483188 | 30174 | 6:36489959 |
| 30175 | 6:36490754 | 30234 | 6:36545679 | 30259 | 6:36568156 | 30261 | 6:36568846 | 30328 | 6:36807893 |
| 30329 | 6:36808225 | 30459 | 6:37029628 | 30462 | 6:37032995 | 30468 | 6:37035541 | 30566 | 6:37095299 |
| 30653 | 6:37151905 | 30655 | 6:37152373 | 30679 | 6:37160005 | 30735 | 6:37172974 | 30736 | 6:37173048 |
| 30748 | 6:37175662 | 30750 | 6:37175773 | 30757 | 6:37176677 | 30772 | 6:37181087 | 30781 | 6:37182773 |
| 30787 | 6:37183901 | 30792 | 6:37189736 | 30794 | 6:37192102 | 30802 | 6:37214389 | 30852 | 6:37251132 |
| 30858 | 6:37262825 | 30921 | 6:37299855 | 31027 | 6:37329948 | 31093 | 6:37365265 | 31180 | 6:37431101 |
| 31284 | 6:37636883 | 31285 | 6:37636900 | 31524 | 6:37936087 | 31846 | 6:38545113 | 31894 | 6:38558946 |
| 31930 | 6:38800398 | 31931 | 6:38830763 | 31935 | 6:38855286 | 31936 | 6:38862311 | 31937 | 6:38862987 |
| 31938 | 6:38875748 | 31940 | 6:38879318 | 31941 | 6:38882135 | 31980 | 6:39565429 | 31983 | 6:39593110 |
| 32001 | 6:39758696 | 32010 | 6:39853149 | 32018 | 6:40036007 | 32020 | 6:40036943 | 32022 | 6:40040343 |
| 32023 | 6:40053452 | 32025 | 6:40058671 | 32030 | 6:40130949 | 32056 | 6:40611279 | 3206 | 6:40707783 |
| 32064 | 6:40708542 | 32065 | 6:40708988 | 32066 | 6:40709098 | 32067 | 6:40709130 | 32068 | 6:40709457 |
| 32069 | 6:40709727 | 32070 | 6:40709846 | 32071 | 6:40711310 | 32072 | 6:40711346 | 32073 | 6:40711409 |
| 32074 | 6:40711422 | 32075 | 6:40711439 | 32076 | 6:40713027 | 32077 | 6:40713351 | 32078 | 6:40713605 |
| 32079 | 6:40713909 | 32080 | 6:40714153 | 32081 | 6:40714938 | 32082 | 6:40715195 | 32083 | 6:40716811 |
| 32084 | 6:40721324 | 32087 | 6:40743678 | 32090 | 6:40770454 | 32091 | 6:40771992 | 32092 | 6:40771993 |
| 32186 | 6:41827472 | 32240 | 6:42833384 | 32249 | 6:42951534 | 32250 | 6:42951582 | 32251 | 6:42951695 |
| 32252 | 6:42951801 | 32253 | 6:42952447 | 32254 | 6:42953604 | 32255 | 6:42954090 | 32256 | 6:42956624 |
| 32257 | 6:42957873 | 32258 | 6:42961867 | 32259 | 6:42963490 | 32260 | 6:42966370 | 32261 | 6:42969572 |
| 32265 | 6:42996149 | 32268 | 6:43024569 | 32269 | 6:43035223 | 32270 | 6:43035520 | 32278 | 6:43178587 |
| 32292 | 6:43450785 | 32322 | 6:43878243 | 32328 | 6:43959607 | 32364 | 6:44686809 | 32417 | 6:44828139 |
| 32510 | 6:45452605 | 32511 | 6:45453963 | 32521 | 6:45702584 | 32555 | 6:46424441 | 32556 | 6:46446420 |
| 32564 | 6:46615384 | 32573 | 6:46883981 | 32575 | 6:46926732 | 32584 | 6:47108047 | 32611 | 6:47785922 |
| 32645 | 6:48798347 | 32726 | 6:50704099 | 32732 | 6:50885777 | 32742 | 6:51188886 | 32764 | 6:51982250 |
| 32769 | 6:52083644 | 32804 | 6:52427412 | 32805 | 6:52431484 | 32806 | 6:52434675 | 32807 | 6:52444382 |
| 32808 | 6:52444842 | 32809 | 6:52451412 | 32819 | 6:52675393 | 32832 | 6:53029143 | 32838 | 6:53151451 |
| 32907 | 6:55106382 | 32908 | 6:55121620 | 32912 | 6:55288741 | 32916 | 6:55337592 | 32921 | 6:55478053 |
| 32922 | 6:55478241 | 32923 | 6:55480385 | 32924 | 6:55481575 | 32925 | 6:55483221 | 32926 | 6:55483692 |
| 32927 | 6:55484196 | 32928 | 6:55484209 | 32929 | 6:55484776 | 32930 | 6:55484984 | 32931 | 6:55484996 |
| 32932 | 6:55485652 | 32933 | 6:55487306 | 32934 | 6:55487659 | 32935 | 6:55487698 | 32936 | 6:55487961 |
| 32937 | 6:55488137 | 32938 | 6:55488439 | 32939 | 6:55498328 | 32945 | 6:55534953 | 33154 | 6:60793645 |
| 33178 | 6:61401990 | 33201 | 6:62075988 | 33220 | 6:62746494 | 33287 | 6:64826176 | 33288 | 6:64860935 |
| 33301 | 6:65368807 | 33320 | 6:65902330 | 33385 | 6:68144658 | 33560 | 6:73271530 | 33568 | 6:73524311 |
| 33587 | 6:74274673 | 33588 | 6:74278129 | 33590 | 6:74325296 | 33595 | 6:74538501 | 33604 | 6:74934981 |
| 33638 | 6:76140833 | 33664 | 6:76838570 | 33669 | 6:76918617 | 33761 | 6:80397556 | 33786 | 6:80984642 |
| 33856 | 6:83284076 | 33857 | 6:83290251 | 33889 | 6:83810111 | 33919 | 6:84577409 | 33945 | 6:85170579 |
| 33958 | 6:85452102 | 33960 | 6:85473180 | 33973 | 6:85798206 | 33990 | 6:86146685 | 34015 | 6:86736214 |
| 34039 | 6:87076207 | 34076 | 6:87751257 | 34095 | 6:88150285 | 34146 | 6:89563672 | 34160 | 6:89750491 |
| 34167 | 6:89906046 | 34223 | 6:90999178 | 34252 | 6:91356781 | 34256 | 6:91397116 | 34311 | 6:92717918 |
| 34325 | 6:93027241 | 34355 | 6:93561742 | 34379 | 6:94081466 | 34380 | 6:94087064 | 34382 | 6:94089501 |
| 34383 | 6:94094573 | 34384 | 6:94096218 | 34385 | 6:94098014 | 34386 | 6:94098380 | 34387 | 6:94098998 |
| 34456 | 6:96190398 | 34499 | 6:97061854 | 34533 | 6:97481556 | 34534 | 6:97481830 | 34535 | 6:97482142 |
| 34536 | 6:97486160 | 34588 | 6:98499066 | 34597 | 6:98688262 | 34647 | 6:100180448 | 34648 | 6:100181539 |
| 34649 | 6:100181706 | 34650 | 6:100181746 | 34651 | 6:100182503 | 34652 | 6:100183086 | 34653 | 6:100183464 |
| 34654 | 6:100185002 | 34655 | 6:100185489 | 34656 | 6:100185949 | 34657 | 6:100186040 | 34658 | 6:100186295 |
| 34673 | 6:100520648 | 34698 | 6:101088719 | 34699 | 6:101089428 | 34765 | 6:102387484 | 34775 | 6:102527385 |
| 34820 | 6:103206312 | 34874 | 6:104209750 | 34913 | 6:105016409 | 34983 | 6:106924221 | 34985 | 6:106930321 |
| 34998 | 6:107232252 | 35017 | 6:107833335 | 35031 | 6:108314278 | 35032 | 6:108316551 | 35035 | 6:108336234 |
| 35040 | 6:108483986 | 35074 | 6:109282710 | 35086 | 6:109549186 | 35112 | 6:110036778 | 35120 | 6:110267946 |
| 35121 | 6:110296111 | 35154 | 6:111027360 | 35170 | 6:111251001 | 35181 | 6:111506757 | 35194 | 6:111752185 |
| 35195 | 6:111753497 | 35196 | 6:111755682 | 35203 | 6:111845284 | 35215 | 6:112274283 | 35225 | 6:112517961 |
| 35254 | 6:112951648 | 35258 | 6:113193897 | 35264 | 6:113278790 | 35269 | 6:113338198 | 35302 | 6:113832391 |
| 35304 | 6:113899018 | 35316 | 6:114099166 | 35317 | 6:114104514 | 35338 | 6:114493111 | 35356 | 6:114802066 |
| 35377 | 6:115232986 | 35397 | 6:115802755 | 35522 | 7:1173379 | 35548 | 7:1688750 | 35608 | 7:2637204 |
| 35647 | 7:3292376 | 35723 | 7:4848657 | 35745 | 7:5160131 | 35746 | 7:5169066 | 35754 | 7:5443639 |
| 35777 | 7:5627311 | 35779 | 7:5637555 | 35814 | 7:6109291 | 35815 | 7:6109369 | 35816 | 7:6110105 |
| 35817 | 7:6110223 | 35818 | 7:6110813 | 35819 | 7:6112482 | 35820 | 7:6112502 | 35821 | 7:6112503 |
| 35822 | 7:6112832 | 35824 | 7:6114838 | 35825 | 7:6118415 | 35826 | 7:6118766 | 35827 | 7:6119488 |
| 35828 | 7:6122989 | 35829 | 7:6123149 | 35830 | 7:6123438 | 35831 | 7:6128021 | 35832 | 7:6130590 |
| 35833 | 7:6130687 | 35834 | 7:6131292 | 35835 | 7:6131297 | 35836 | 7:6132484 | 35837 | 7:6132747 |
| 35838 | 7:6133083 | 35840 | 7:6137471 | 35854 | 7:6339944 | 35855 | 7:6341414 | 35856 | 7:6341470 |
| 35857 | 7:6341501 | 35858 | 7:6345398 | 35860 | 7:6348000 | 35861 | 7:6348353 | 35862 | 7:6352211 |
| 35863 | 7:6356100 | 35864 | 7:6356608 | 35865 | 7:6356670 | 35881 | 7:6710471 | 35894 | 7:6824241 |
| 35903 | 7:6999017 | 35914 | 7:7143257 | 35929 | 7:7557725 | 35930 | 7:7562490 | 35933 | 7:7584113 |
| 35959 | 7:8549050 | 35966 | 7:8914661 | 35977 | 7:9686485 | 35978 | 7:9760724 | 36006 | 7:11067502 |
| 36013 | 7:11123621 | 36031 | 7:11430993 | 36036 | 7:11668056 | 36046 | 7:11952515 | 36081 | 7:12499238 |
| 36157 | 7:13717835 | 36185 | 7:14182114 | 36243 | 7:15057296 | 36303 | 7:16284081 | 36325 | 7:16641516 |
| 36363 | 7:17636616 | 36365 | 7:17641071 | 36384 | 7:18214147 | 36409 | 7:18665015 | 36416 | 7:18849856 |
| 36433 | 7:19227351 | 36442 | 7:19317690 | 36444 | 7:19333133 | 36461 | 7:19721740 | 36516 | 7:20334637 |
| 36524 | 7:20395009 | 36536 | 7:20701671 | 36537 | 7:20704005 | 36539 | 7:20747147 | 36548 | 7:20935768 |
| 36639 | 7:23007875 | 36650 | 7:23297694 | 36651 | 7:23350564 | 36657 | 7:23528238 | 36658 | 7:23528885 |
| 36684 | 7:24067558 | 36692 | 7:24236323 | 36709 | 7:24746921 | 36734 | 7:25342888 | 36737 | 7:25403017 |
| 36843 | 7:27517669 | 36845 | 7:27577715 | 36846 | 7:27588151 | 36892 | 7:28685778 | 36910 | 7:29075991 |
| 36945 | 7:29836572 | 36952 | 7:29934618 | 36955 | 7:30045346 | 36969 | 7:30374597 | 36979 | 7:30662217 |
| 37046 | 7:31780219 | 37055 | 7:32039528 | 37090 | 7:33006143 | 37103 | 7:33314191 | 37140 | 7:34167737 |
| 37157 | 7:34561935 | 37166 | 7:34800911 | 37204 | 7:35829441 | 37224 | 7:36375051 | 37225 | 7:36381643 |
| 37271 | 7:37512158 | 37358 | 7:39431184 | 37383 | 7:40018615 | 37386 | 7:40105218 | 37387 | 7:40106188 |
| 37403 | 7:40313129 | 37421 | 7:40543374 | 37501 | 7:42717113 | 37502 | 7:42793836 | 37562 | 7:44383968 |
| 37577 | 7:44807464 | 37579 | 7:44874918 | 37589 | 7:45086131 | 37597 | 7:45264623 | 37598 | 7:45268714 |
| 37606 | 7:45462352 | 37628 | 7:46197379 | 37629 | 7:46207779 | 37647 | 7:46698742 | 37652 | 7:46763773 |
| 37665 | 7:47007606 | 37666 | 7:47009580 | 37667 | 7:47010093 | 37668 | 7:47010163 | 37669 | 7:47011323 |
| 37670 | 7:47011475 | 37671 | 7:47012085 | 37672 | 7:47012699 | 37674 | 7:47017310 | 37675 | 7:47018943 |
| 37676 | 7:47021031 | 37716 | 7:48370104 | 37736 | 7:48785206 | 37758 | 7:49338040 | 37807 | 7:51678528 |
| 37832 | 7:52217990 | 37892 | 7:52801631 | 37901 | 7:53051717 | 37903 | 7:53072922 | 37907 | 7:53192775 |
| 37929 | 7:53916384 | 37998 | 7:55634474 | 38004 | 7:55875367 | 38018 | 7:56132046 | 38036 | 7:56553298 |
| 38044 | 7:56873627 | 38072 | 7:57843543 | 38089 | 7:58267508 | 38091 | 7:58338126 | 38139 | 7:59331366 |
| 38146 | 7:59368692 | 38166 | 7:59579502 | 38249 | 7:61303310 | 38261 | 7:61485578 | 38264 | 7:61501849 |
| 38305 | 7:62532423 | 38306 | 7:62554501 | 38309 | 7:62558404 | 38377 | 7:63949067 | 38388 | 7:64285750 |
| 38397 | 7:64625175 | 38412 | 7:64930029 | 38413 | 7:64937658 | 38420 | 7:65072560 | 38432 | 7:65258702 |
| 38507 | 7:66460989 | 38538 | 7:67323191 | 38555 | 7:67781532 | 38557 | 7:67828291 | 38563 | 7:67940525 |
| 38586 | 7:68625153 | 38589 | 7:68641820 | 38642 | 7:69866274 | 38644 | 7:69879505 | 38646 | 7:69905767 |
| 38653 | 7:70037542 | 38655 | 7:70093469 | 38656 | 7:70099250 | 38666 | 7:70331724 | 38669 | 7:70419871 |
| 38689 | 7:71130508 | 38698 | 7:71415976 | 38716 | 7:71712692 | 38717 | 7:71727860 | 38722 | 7:71825182 |
| 38723 | 7:71830368 | 38725 | 7:71921897 | 38754 | 7:72753552 | 38794 | 7:74007645 | 38826 | 7:74850810 |
| 38869 | 7:75797751 | 38879 | 7:76006096 | 38880 | 7:76006417 | 38881 | 7:76010723 | 38916 | 7:76940482 |
| 38930 | 7:77150855 | 38941 | 7:77453724 | 38950 | 7:77656774 | 38964 | 7:77949425 | 38989 | 7:78379223 |
| 39000 | 7:78748906 | 39008 | 7:79016201 | 39009 | 7:79016267 | 39010 | 7:79016787 | 39011 | 7:79017056 |
| 39012 | 7:79017378 | 39013 | 7:79017385 | 39014 | 7:79017772 | 39015 | 7:79017959 | 39016 | 7:79018035 |
| 39017 | 7:79018571 | 39018 | 7:79018616 | 39019 | 7:79018620 | 39020 | 7:79019988 | 39021 | 7:79023682 |
| 39022 | 7:79025120 | 39023 | 7:79025266 | 39024 | 7:79041811 | 39025 | 7:79042476 | 39027 | 7:79048979 |
| 39028 | 7:79070660 | 39029 | 7:79071063 | 39031 | 7:79135260 | 39032 | 7:79135270 | 39040 | 7:79367716 |
| 39056 | 7:79660008 | 39076 | 7:80098520 | 39077 | 7:80104490 | 39096 | 7:80463366 | 39111 | 7:80573664 |
| 39122 | 7:80835359 | 39133 | 7:81097721 | 39221 | 7:82912666 | 39241 | 7:83502868 | 39259 | 7:83929718 |
| 39299 | 7:85098423 | 39310 | 7:85314414 | 39315 | 7:85586461 | 39383 | 7:87496719 | 39413 | 7:88462551 |
| 39425 | 7:88705722 | 39622 | 7:91895030 | 39706 | 7:94524953 | 39712 | 7:94671798 | 39713 | 7:94672003 |
| 39714 | 7:94688436 | 39715 | 7:94691325 | 39728 | 7:95018455 | 39768 | 7:95975870 | 39773 | 7:96088093 |
| 39774 | 7:96093182 | 39795 | 7:96175080 | 39796 | 7:96175132 | 39797 | 7:96175168 | 39798 | 7:96175341 |
| 39799 | 7:96175357 | 39800 | 7:96175409 | 39801 | 7:96175451 | 39802 | 7:96175628 | 39803 | 7:96176143 |
| 39804 | 7:96176528 | 39805 | 7:96176908 | 39809 | 7:96193901 | 39812 | 7:96199226 | 39815 | 7:96205718 |
| 39816 | 7:96205942 | 39817 | 7:96206884 | 39818 | 7:96207458 | 39819 | 7:96207882 | 39820 | 7:96208060 |
| 39821 | 7:96208106 | 39822 | 7:96208582 | 39823 | 7:96208995 | 39824 | 7:96209096 | 39825 | 7:96209862 |
| 39826 | 7:96211592 | 39827 | 7:96211829 | 39828 | 7:96216996 | 39832 | 7:96221555 | 39833 | 7:96223919 |
| 39834 | 7:96228261 | 39835 | 7:96228951 | 39836 | 7:96231447 | 39837 | 7:96235988 | 39838 | 7:96236371 |
| 39839 | 7:96236926 | 39840 | 7:96239015 | 39841 | 7:96239023 | 39842 | 7:96242837 | 39843 | 7:96246776 |
| 39844 | 7:96246937 | 39845 | 7:96248619 | 39846 | 7:96248835 | 39847 | 7:96249753 | 39848 | 7:96249787 |
| 39849 | 7:96249862 | 39850 | 7:96254816 | 39851 | 7:96263665 | 39852 | 7:96264122 | 39853 | 7:96264141 |
| 39854 | 7:96265124 | 39855 | 7:96265144 | 39856 | 7:96265161 | 39857 | 7:96265371 | 39858 | 7:96265379 |
| 39860 | 7:96271576 | 39861 | 7:96271653 | 39862 | 7:96273181 | 39863 | 7:96273246 | 39864 | 7:96273247 |
| 39865 | 7:96273562 | 39866 | 7:96273613 | 39867 | 7:96274725 | 39869 | 7:96291284 | 39870 | 7:96291379 |
| 39871 | 7:96291389 | 39872 | 7:96291500 | 39873 | 7:96291530 | 39874 | 7:96293330 | 39875 | 7:96293559 |
| 39876 | 7:96293758 | 39877 | 7:96293861 | 39878 | 7:96295220 | 39879 | 7:96295518 | 39880 | 7:96295778 |
| 39881 | 7:96295878 | 39882 | 7:96295906 | 39883 | 7:96295937 | 39884 | 7:96296075 | 39885 | 7:96296087 |
| 39886 | 7:96296169 | 39887 | 7:96296233 | 39888 | 7:96296513 | 39889 | 7:96296531 | 39890 | 7:96296546 |
| 39891 | 7:96296683 | 39892 | 7:96297463 | 39893 | 7:96297555 | 39894 | 7:96297857 | 39895 | 7:96298677 |
| 39896 | 7:96299647 | 39897 | 7:96300501 | 39898 | 7:96303921 | 39899 | 7:96304204 | 39900 | 7:96305341 |
| 39901 | 7:96305698 | 39902 | 7:96307964 | 39903 | 7:96308750 | 39904 | 7:96308945 | 39905 | 7:96309033 |
| 39906 | 7:96310180 | 39907 | 7:96311062 | 39909 | 7:96314531 | 39910 | 7:96314540 | 39913 | 7:96318724 |
| 39914 | 7:96322543 | 39915 | 7:96322732 | 39916 | 7:96323877 | 39917 | 7:96323928 | 39918 | 7:96324360 |
| 39919 | 7:96330432 | 39920 | 7:96331236 | 39921 | 7:96331603 | 39922 | 7:96333195 | 39923 | 7:96333277 |
| 39924 | 7:96333341 | 39925 | 7:96333563 | 39926 | 7:96333575 | 39927 | 7:96334610 | 39928 | 7:96334702 |
| 39929 | 7:96334763 | 39930 | 7:96335706 | 39931 | 7:96337614 | 39933 | 7:96354116 | 39935 | 7:96355876 |
| 39936 | 7:96358329 | 39937 | 7:96361391 | 39938 | 7:96363115 | 39940 | 7:96374561 | 39949 | 7:96519372 |
| 39953 | 7:96606759 | 40022 | 7:98399080 | 40043 | 7:99244131 | 40046 | 7:99254092 | 40085 | 7:100105110 |
| 40106 | 7:100563689 | 40108 | 7:100578900 | 40112 | 7:100659403 | 40127 | 7:101215525 | 40166 | 7:102357637 |
| 40201 | 7:103052550 | 40216 | 7:103329082 | 40224 | 7:103487103 | 40229 | 7:103675594 | 40232 | 7:103729325 |
| 40239 | 7:103982179 | 40240 | 7:103982180 | 40241 | 7:103982288 | 40242 | 7:103982423 | 40243 | 7:103985377 |
| 40253 | 7:104230973 | 40291 | 7:105373160 | 40294 | 7:105399311 | 40296 | 7:105442182 | 40297 | 7:105515412 |
| 40324 | 7:106146247 | 40372 | 7:107094209 | 40415 | 7:108495274 | 40496 | 8:356371 | 40500 | 8:395127 |
| 40585 | 8:2113854 | 40598 | 8:2387478 | 40600 | 8:2420577 | 40637 | 8:3508161 | 40640 | 8:3527178 |
| 40645 | 8:3640550 | 40647 | 8:3666778 | 40718 | 8:5496138 | 40732 | 8:5839173 | 40736 | 8:5906023 |
| 40751 | 8:6327904 | 40804 | 8:7593827 | 40861 | 8:8914108 | 40865 | 8:9015228 | 40882 | 8:9392626 |
| 40960 | 8:11244122 | 40966 | 8:11330242 | 40968 | 8:11403633 | 40975 | 8:11522513 | 40984 | 8:11668581 |
| 40991 | 8:11856184 | 40997 | 8:12018134 | 41009 | 8:12363854 | 41050 | 8:13811430 | 41095 | 8:15117985 |
| 41099 | 8:15188506 | 41110 | 8:15477698 | 41112 | 8:15494200 | 41118 | 8:15553158 | 41119 | 8:15574036 |
| 41126 | 8:15701758 | 41137 | 8:16168316 | 41138 | 8:16168775 | 41201 | 8:17444173 | 41211 | 8:17638060 |
| 41227 | 8:18138392 | 41250 | 8:18931382 | 41279 | 8:19741400 | 41290 | 8:19953078 | 41331 | 8:20945241 |
| 41343 | 8:21351226 | 41403 | 8:23473039 | 41410 | 8:23722262 | 41451 | 8:24658012 | 41460 | 8:24730121 |
| 41567 | 8:25718774 | 41578 | 8:25952516 | 41581 | 8:26020143 | 41607 | 8:26647199 | 41617 | 8:26902337 |
| 41618 | 8:26902597 | 41619 | 8:26902692 | 41620 | 8:26906751 | 41621 | 8:26909158 | 41622 | 8:26909371 |
| 41663 | 8:28392311 | 41694 | 8:29260025 | 41703 | 8:29463858 | 41797 | 8:31800228 | 41798 | 8:31843921 |
| 41829 | 8:32784569 | 41840 | 8:33342641 | 41853 | 8:33651679 | 41868 | 8:34125341 | 41876 | 8:34286689 |
| 41883 | 8:34490228 | 41897 | 8:34815302 | 41907 | 8:35172469 | 41963 | 8:36234097 | 41982 | 8:36725916 |
| 41989 | 8:36958536 | 42015 | 8:37563030 | 42052 | 8:38794675 | 42072 | 8:39672661 | 42134 | 8:40784197 |
| 42138 | 8:40885929 | 42141 | 8:41023065 | 42185 | 8:42062463 | 42259 | 8:44042188 | 42291 | 8:45099245 |
| 42293 | 8:45139392 | 42318 | 8:45729304 | 42324 | 8:45797263 | 42335 | 8:46074143 | 42336 | 8:46102241 |
| 42337 | 8:46116001 | 42338 | 8:46116354 | 42339 | 8:46135520 | 42341 | 8:46137990 | 42342 | 8:46144578 |
| 42344 | 8:46153342 | 42345 | 8:46156573 | 42375 | 8:46982936 | 42420 | 8:48031781 | 42430 | 8:48479991 |
| 42539 | 8:51426634 | 42545 | 8:51576820 | 42575 | 8:52252692 | 42585 | 8:52524895 | 42602 | 8:52976427 |
| 42613 | 8:53298802 | 42616 | 8:53382084 | 42702 | 8:55675444 | 42724 | 8:56364736 | 42725 | 8:56366787 |
| 42751 | 8:56934422 | 42753 | 8:56941050 | 42754 | 8:57019107 | 42783 | 8:57597865 | 42810 | 8:58618980 |
| 42832 | 8:59315871 | 42871 | 8:60125622 | 42934 | 8:61718348 | 42938 | 8:61889487 | 42971 | 8:62566075 |
| 42972 | 8:62584738 | 42992 | 8:63183224 | 43052 | 8:64941395 | 43080 | 8:65506614 | 43107 | 8:66205907 |
| 43118 | 8:66471868 | 43138 | 8:66967439 | 43144 | 8:67016183 | 43176 | 8:67787815 | 43178 | 8:67793980 |
| 43188 | 8:67954514 | 43191 | 8:68086072 | 43206 | 8:68512363 | 43207 | 8:68517559 | 43214 | 8:68740750 |
| 43231 | 8:69106674 | 43234 | 8:69118901 | 43266 | 8:70045486 | 43271 | 8:70141611 | 43282 | 8:70418020 |
| 43313 | 8:71060104 | 43314 | 8:71090353 | 43325 | 8:71516540 | 43350 | 8:72260036 | 43357 | 8:72432728 |
| 43358 | 8:72437508 | 43365 | 8:72557656 | 43388 | 8:73136540 | 43419 | 8:73847683 | 43451 | 8:74692744 |
| 43498 | 8:76193941 | 43517 | 8:76534193 | 43528 | 8:76736188 | 43587 | 8:78346120 | 43592 | 8:78443361 |
| 43628 | 8:79886867 | 43672 | 8:81022175 | 43675 | 8:81050611 | 43723 | 8:82076199 | 43741 | 8:82402914 |
| 43744 | 8:82410990 | 43754 | 8:82684234 | 43775 | 8:83245271 | 43786 | 8:83673295 | 43800 | 8:83846337 |
| 43806 | 8:83935130 | 43889 | 8:86085819 | 43893 | 8:86142301 | 43906 | 8:86539317 | 43913 | 8:86746201 |
| 43924 | 8:86977892 | 43928 | 8:87127829 | 43929 | 8:87139627 | 43930 | 8:87141766 | 43931 | 8:87142536 |
| 43933 | 8:87168896 | 43934 | 8:87175091 | 43935 | 8:87177155 | 43936 | 8:87183548 | 43937 | 8:87200658 |
| 43938 | 8:87218682 | 43946 | 8:87439832 | 44026 | 8:89300462 | 44066 | 8:90333835 | 44083 | 8:90850582 |
| 44093 | 8:90959304 | 44126 | 8:91835974 | 44162 | 8:92493864 | 44184 | 8:93235330 | 44287 | 8:96565770 |
| 44307 | 8:97085820 | 44344 | 8:98025826 | 44346 | 8:98099120 | 44377 | 8:98925263 | 44383 | 8:99073097 |
| 44444 | 8:100321613 | 44447 | 8:100390505 | 44521 | 8:102187006 | 44544 | 8:102423408 | 44548 | 8:102462371 |
| 44563 | 8:102641248 | 44565 | 8:102643183 | 44575 | 8:102804781 | 44617 | 8:103596897 | 44665 | 8:104455217 |
| 44671 | 8:104552362 | 44698 | 8:105230189 | 44708 | 8:105413829 | 44750 | 8:106646055 | 44751 | 8:106656709 |
| 44775 | 8:107216243 | 44777 | 8:107231955 | 44791 | 8:107578002 | 44798 | 8:107830676 | 44819 | 8:108486026 |
| 44820 | 8:108568498 | 44825 | 8:108815568 | 44854 | 8:109956440 | 44867 | 8:110340060 | 44885 | 8:110872351 |
| 44889 | 8:110978542 | 44927 | 9:28156 | 44939 | 9:575925 | 44940 | 9:575959 | 44942 | 9:582062 |
| 44943 | 9:590791 | 44944 | 9:593091 | 44945 | 9:600069 | 44946 | 9:600393 | 44948 | 9:618129 |
| 44949 | 9:632185 | 44950 | 9:633412 | 44951 | 9:642243 | 44952 | 9:659431 | 44953 | 9:659947 |
| 44954 | 9:660978 | 44955 | 9:661495 | 44956 | 9:665906 | 44958 | 9:681361 | 44959 | 9:697171 |
| 44960 | 9:708137 | 44961 | 9:709297 | 44964 | 9:726333 | 44969 | 9:900985 | 45000 | 9:1570889 |
| 45010 | 9:1844977 | 45023 | 9:2164529 | 45053 | 9:3003204 | 45069 | 9:3571118 | 45103 | 9:4701519 |
| 45114 | 9:5085846 | 45144 | 9:5897942 | 45145 | 9:5911158 | 45148 | 9:6056118 | 45160 | 9:6416188 |
| 45193 | 9:7630506 | 45195 | 9:7644292 | 45210 | 9:7983021 | 45214 | 9:8111645 | 45256 | 9:9020374 |
| 45257 | 9:9029730 | 45403 | 9:11584044 | 45408 | 9:11752659 | 45448 | 9:12626683 | 45454 | 9:12671087 |
| 45455 | 9:12689678 | 45457 | 9:12695431 | 45458 | 9:12695934 | 45459 | 9:12695941 | 45460 | 9:12696018 |
| 45461 | 9:12696113 | 45462 | 9:12696251 | 45463 | 9:12696292 | 45464 | 9:12696440 | 45465 | 9:12696661 |
| 45466 | 9:12698815 | 45467 | 9:12698843 | 45468 | 9:12700184 | 45472 | 9:12770979 | 45479 | 9:13069374 |
| 45480 | 9:13082121 | 45482 | 9:13102812 | 45483 | 9:13103635 | 45484 | 9:13104067 | 45486 | 9:13108898 |
| 45487 | 9:13109246 | 45488 | 9:13109258 | 45489 | 9:13110088 | 45494 | 9:13242607 | 45533 | 9:14240906 |
| 45547 | 9:14661048 | 45581 | 9:15193969 | 45591 | 9:15445642 | 45614 | 9:15671238 | 45615 | 9:15672381 |
| 45616 | 9:15672435 | 45617 | 9:15672993 | 45618 | 9:15673156 | 45619 | 9:15673271 | 45620 | 9:15673402 |
| 45621 | 9:15674725 | 45632 | 9:15869515 | 45635 | 9:15935953 | 45639 | 9:15995486 | 45654 | 9:16430498 |
| 45663 | 9:16688073 | 45693 | 9:17598783 | 45694 | 9:17599118 | 45783 | 9:20470091 | 45787 | 9:20622642 |
| 45794 | 9:20764203 | 45820 | 9:21613566 | 45821 | 9:21636143 | 45822 | 9:21640871 | 45823 | 9:21643825 |
| 45825 | 9:21654717 | 45826 | 9:21683117 | 45827 | 9:21683679 | 45829 | 9:21715481 | 45830 | 9:21740090 |
| 45831 | 9:21757906 | 45855 | 9:22294337 | 45866 | 9:22593668 | 45869 | 9:22639958 | 45923 | 9:23824584 |
| 45927 | 9:23893279 | 45955 | 9:24560985 | 45994 | 9:25737610 | 45995 | 9:25738079 | 45996 | 9:25741377 |
| 46014 | 9:26124495 | 46025 | 9:26374532 | 46056 | 9:27294641 | 46078 | 9:27733478 | 46081 | 9:27779272 |
| 46083 | 9:27801667 | 46103 | 9:28362224 | 46104 | 9:28363030 | 46113 | 9:28567312 | 46114 | 9:28568804 |
| 46115 | 9:28569972 | 46116 | 9:28591418 | 46117 | 9:28593815 | 46122 | 9:28646308 | 46140 | 9:29091195 |
| 46143 | 9:29192292 | 46144 | 9:29204130 | 46145 | 9:29256950 | 46147 | 9:29283806 | 46148 | 9:29295585 |
| 46151 | 9:29380299 | 46152 | 9:29380683 | 46153 | 9:29381237 | 46154 | 9:29381255 | 46155 | 9:29383215 |
| 46156 | 9:29383228 | 46157 | 9:29383283 | 46158 | 9:29384303 | 46159 | 9:29384799 | 46160 | 9:29385786 |
| 46161 | 9:29385818 | 46162 | 9:29386059 | 46163 | 9:29386376 | 46164 | 9:29387098 | 46166 | 9:29400041 |
| 46167 | 9:29400182 | 46168 | 9:29402441 | 46169 | 9:29402650 | 46170 | 9:29403623 | 46171 | 9:29409271 |
| 46172 | 9:29411199 | 46187 | 9:29691912 | 46188 | 9:29692738 | 46199 | 9:30032893 | 46200 | 9:30033128 |
| 46218 | 9:30451686 | 46223 | 9:30612426 | 46225 | 9:30625188 | 46246 | 9:31013461 | 46311 | 9:32638638 |
| 46346 | 9:33419705 | 46367 | 9:33960698 | 46387 | 9:34556125 | 46410 | 9:35190440 | 46444 | 9:36105021 |
| 46494 | 9:37158301 | 46519 | 9:37870307 | 46552 | 9:38368263 | 46582 | 9:38867506 | 46583 | 9:38875675 |
| 46584 | 9:38878148 | 46586 | 9:38894269 | 46637 | 9:39995374 | 46695 | 9:41446116 | 46699 | 9:41532669 |
| 46739 | 9:42551675 | 46776 | 9:43626998 | 46784 | 9:43912263 | 46785 | 9:43915403 | 46798 | 9:44230197 |
| 46806 | 9:44371483 | 46812 | 9:44533430 | 46816 | 9:44595447 | 46859 | 9:46082473 | 46887 | 9:46922739 |
| 46902 | 9:47520187 | 46903 | 9:47553422 | 46907 | 9:47590125 | 46975 | 9:49674165 | 46989 | 9:50119411 |
| 47011 | 9:50615961 | 47022 | 9:50886609 | 47024 | 9:50944146 | 47064 | 9:52161063 | 47071 | 9:52377348 |
| 47106 | 9:53217250 | 47107 | 9:53217253 | 47108 | 9:53217815 | 47109 | 9:53217988 | 47110 | 9:53225739 |
| 47111 | 9:53226122 | 47112 | 9:53226145 | 47113 | 9:53227113 | 47114 | 9:53234782 | 47115 | 9:53245948 |
| 47116 | 9:53269708 | 47118 | 9:53277095 | 47119 | 9:53298978 | 47163 | 9:54707994 | 47167 | 9:54805840 |
| 47180 | 9:55233499 | 47206 | 9:56084986 | 47274 | 9:57805504 | 47290 | 9:58371472 | 47310 | 9:58992635 |
| 47323 | 9:59401110 | 47324 | 9:59402335 | 47327 | 9:59422003 | 47329 | 9:59459837 | 47373 | 9:60362161 |
| 47469 | 9:63010039 | 47474 | 9:63179604 | 47477 | 9:63266632 | 47524 | 9:64700448 | 47593 | 9:65489746 |
| 47601 | 9:65582850 | 47619 | 9:66094001 | 47639 | 9:66606853 | 47652 | 9:67044823 | 47659 | 9:67241727 |
| 47664 | 9:67366039 | 47677 | 9:67697561 | 47689 | 9:68036049 | 47693 | 9:68114580 | 47694 | 9:68115586 |
| 47713 | 9:68594580 | 47753 | 9:69635715 | 47757 | 9:69752727 | 47788 | 9:70415891 | 47795 | 9:70619172 |
| 47807 | 9:70987356 | 47808 | 9:70997161 | 47878 | 9:72597976 | 47889 | 9:72874182 | 47936 | 9:74030455 |
| 47949 | 9:74381423 | 47965 | 9:74748032 | 48044 | 9:76966345 | 48070 | 9:77485895 | 48073 | 9:77511368 |
| 48075 | 9:77523509 | 48078 | 9:77562662 | 48081 | 9:77694221 | 48083 | 9:77699388 | 48084 | 9:77713754 |
| 48085 | 9:77715782 | 48086 | 9:77716409 | 48087 | 9:77716687 | 48088 | 9:77717203 | 48089 | 9:77717466 |
| 48090 | 9:77718722 | 48092 | 9:77723623 | 48093 | 9:77728652 | 48094 | 9:77728956 | 48095 | 9:77729189 |
| 48096 | 9:77729397 | 48097 | 9:77729413 | 48098 | 9:77730079 | 48099 | 9:77730486 | 48100 | 9:77730739 |
| 48101 | 9:77730950 | 48102 | 9:77731339 | 48103 | 9:77731548 | 48104 | 9:77731587 | 48105 | 9:77731649 |
| 48106 | 9:77732824 | 48107 | 9:77733602 | 48108 | 9:77733985 | 48109 | 9:77734457 | 48110 | 9:77734537 |
| 48111 | 9:77734632 | 48112 | 9:77739942 | 48113 | 9:77740756 | 48114 | 9:77741688 | 48115 | 9:77742632 |
| 48116 | 9:77743840 | 48117 | 9:77744601 | 48118 | 9:77744620 | 48119 | 9:77745039 | 48120 | 9:77745584 |
| 48121 | 9:77745608 | 48122 | 9:77751329 | 48123 | 9:77751755 | 48124 | 9:77754808 | 48125 | 9:77756169 |
| 48126 | 9:77756446 | 48127 | 9:77756531 | 48128 | 9:77762645 | 48129 | 9:77764362 | 48132 | 9:77792668 |
| 48133 | 9:77804673 | 48134 | 9:77811111 | 48135 | 9:77811220 | 48136 | 9:77811579 | 48137 | 9:77811594 |
| 48138 | 9:77811612 | 48139 | 9:77811647 | 48140 | 9:77811795 | 48141 | 9:77812291 | 48142 | 9:77812385 |
| 48143 | 9:77812527 | 48144 | 9:77812542 | 48145 | 9:77812555 | 48146 | 9:77812585 | 48191 | 9:79533173 |
| 48196 | 9:79702375 | 48249 | 9:80821611 | 48264 | 9:81093450 | 48265 | 9:81096096 | 48282 | 9:81398472 |
| 48298 | 9:81743991 | 48302 | 9:81797320 | 48321 | 9:82185927 | 48326 | 9:82275767 | 48341 | 9:82639891 |
| 48346 | 9:82675266 | 48347 | 9:82681644 | 48349 | 9:82685499 | 48350 | 9:82696652 | 48351 | 9:82697299 |
| 48352 | 9:82699229 | 48353 | 9:82699333 | 48355 | 9:82706831 | 48356 | 9:82708646 | 48366 | 9:82981739 |
| 48375 | 9:83266367 | 48408 | 9:84082840 | 48409 | 9:84084042 | 48410 | 9:84084459 | 48411 | 9:84098275 |
| 48412 | 9:84100341 | 48413 | 9:84101062 | 48419 | 9:84280123 | 48430 | 9:84602199 | 48453 | 9:85206226 |
| 48527 | 9:86728832 | 48542 | 9:87041634 | 48554 | 9:87334131 | 48645 | 9:88942711 | 48697 | 9:89633025 |
| 48704 | 9:89813410 | 48724 | 9:90315206 | 48740 | 9:90776042 | 48765 | 9:91141962 | 48791 | 9:91739734 |
| 48805 | 9:92056243 | 48807 | 9:92137059 | 48974 | 9:95252173 | 48981 | 9:95442777 | 49012 | 9:96258094 |
| 49084 | 9:97052004 | 49086 | 9:97068291 | 49093 | 9:97195333 | 49115 | 9:97614153 | 49118 | 9:97651483 |
| 49158 | 9:98444344 | 49165 | 9:98565031 | 49183 | 9:99022247 | 49194 | 9:99214577 | 49227 | 9:99757934 |
| 49228 | 9:99766501 | 49286 | 9:100838600 | 49287 | 9:100865957 | 49301 | 9:101130910 | 49308 | 9:101233972 |
| 49316 | 9:101405391 | 49323 | 9:101487283 | 49399 | 9:102585986 | 49428 | 9:102905463 | 49443 | 9:103267046 |
| 49470 | 9:103818635 | 49557 | 10:1514567 | 49560 | 10:1608093 | 49571 | 10:1809005 | 49573 | 10:1812942 |
| 49575 | 10:1815628 | 49596 | 10:2181089 | 49600 | 10:2318770 | 49603 | 10:2355906 | 49633 | 10:2956009 |
| 49650 | 10:3286732 | 49685 | 10:3957621 | 49719 | 10:4637364 | 49756 | 10:5504897 | 49757 | 10:5506239 |
| 49758 | 10:5506479 | 49759 | 10:5506633 | 49760 | 10:5507158 | 49761 | 10:5507326 | 49762 | 10:5507349 |
| 49763 | 10:5507472 | 49764 | 10:5507500 | 49765 | 10:5507611 | 49766 | 10:5507931 | 49767 | 10:5507997 |
| 49768 | 10:5508125 | 49769 | 10:5508230 | 49770 | 10:5509205 | 49771 | 10:5509288 | 49772 | 10:5511487 |
| 49773 | 10:5511551 | 49774 | 10:5511726 | 49817 | 10:6459980 | 49835 | 10:6823353 | 49837 | 10:6940126 |
| 49899 | 10:8330854 | 49901 | 10:8385791 | 49917 | 10:8566513 | 49965 | 10:9790239 | 49972 | 10:9846509 |
| 49987 | 10:10261088 | 50046 | 10:11321931 | 50047 | 10:11322196 | 50076 | 10:12039647 | 50090 | 10:12326160 |
| 50091 | 10:12337486 | 50093 | 10:12394358 | 50099 | 10:12500777 | 50121 | 10:13059560 | 50124 | 10:13141850 |
| 50132 | 10:13207866 | 50133 | 10:13222364 | 50134 | 10:13224497 | 50135 | 10:13225359 | 50136 | 10:13226008 |
| 50139 | 10:13238081 | 50140 | 10:13248003 | 50142 | 10:13255208 | 50143 | 10:13258421 | 50144 | 10:13282620 |
| 50145 | 10:13286682 | 50149 | 10:13347534 | 50150 | 10:13347535 | 50179 | 10:13823541 | 50183 | 10:13888930 |
| 50189 | 10:13964255 | 50193 | 10:14079328 | 50212 | 10:14629981 | 50215 | 10:14653991 | 50257 | 10:15551922 |
| 50260 | 10:15625431 | 50298 | 10:16237750 | 50328 | 10:16751980 | 50350 | 10:17119224 | 50352 | 10:17147669 |
| 50367 | 10:17432966 | 50379 | 10:17732565 | 50396 | 10:18087252 | 50397 | 10:18089168 | 50398 | 10:18089791 |
| 50399 | 10:18089811 | 50400 | 10:18092432 | 50401 | 10:18095170 | 50402 | 10:18096798 | 50403 | 10:18100952 |
| 50404 | 10:18105123 | 50411 | 10:18246343 | 50458 | 10:19161271 | 50477 | 10:19610258 | 50508 | 10:20302691 |
| 50514 | 10:20365815 | 50579 | 10:21377001 | 50615 | 10:22113385 | 50620 | 10:22271591 | 50709 | 10:26380149 |
| 50714 | 10:26474079 | 50715 | 10:26474882 | 50741 | 10:26820943 | 50777 | 10:27459845 | 50848 | 10:28936817 |
| 50850 | 10:28939598 | 50861 | 10:29148690 | 50903 | 10:29990871 | 50973 | 10:32272335 | 50977 | 10:32347626 |
| 50980 | 10:32449180 | 50984 | 10:32556195 | 51013 | 10:33127535 | 51014 | 10:33187873 | 51029 | 10:33566653 |
| 51086 | 10:34900072 | 51089 | 10:34926072 | 51095 | 10:35028845 | 51152 | 10:36423193 | 51178 | 10:37020667 |
| 51199 | 10:37655975 | 51203 | 10:37686916 | 51241 | 10:38641559 | 51258 | 10:39024264 | 51342 | 10:41413651 |
| 51351 | 10:41730162 | 51352 | 10:41731424 | 51359 | 10:41898236 | 51375 | 10:42389264 | 51378 | 10:42430118 |
| 51380 | 10:42448787 | 51382 | 10:42452483 | 51383 | 10:42455131 | 51389 | 10:42580900 | 51404 | 10:43179077 |
| 51419 | 10:43825008 | 51479 | 10:45276327 | 51526 | 10:46079546 | 51575 | 10:47154407 | 51602 | 10:47651824 |
| 51624 | 10:48309146 | 51656 | 10:48956010 | 51683 | 10:49439054 | 51714 | 10:50089231 | 51803 | 10:52130146 |
| 51819 | 10:52557986 | 51853 | 10:53459983 | 51878 | 10:54196187 | 51893 | 10:54537058 | 51894 | 10:54542851 |
| 51931 | 10:55708254 | 51954 | 10:56299639 | 51978 | 10:56721716 | 51981 | 10:56843229 | 51982 | 10:56922663 |
| 51988 | 10:57043405 | 51991 | 10:57115514 | 52018 | 10:57976067 | 52081 | 10:59624803 | 52102 | 10:60442020 |
| 52108 | 10:60636369 | 52159 | 10:62050176 | 52174 | 10:62290589 | 52249 | 10:64233969 | 52267 | 10:64525088 |
| 52268 | 10:64536219 | 52269 | 10:64543768 | 52270 | 10:64548032 | 52271 | 10:64548906 | 52286 | 10:64979230 |
| 52295 | 10:65266598 | 52339 | 10:66097225 | 52340 | 10:66120437 | 52361 | 10:66674133 | 52362 | 10:66674580 |
| 52363 | 10:66675048 | 52364 | 10:66675130 | 52366 | 10:66770244 | 52422 | 10:68107692 | 52424 | 10:68140093 |
| 52444 | 10:68518650 | 52536 | 10:71021997 | 52557 | 10:71661492 | 52574 | 10:72103748 | 52600 | 10:73153480 |
| 52604 | 10:73217430 | 52615 | 10:73492848 | 52620 | 10:73620458 | 52681 | 10:75374747 | 52696 | 10:75774470 |
| 52699 | 10:75886187 | 52707 | 10:76141481 | 52708 | 10:76174432 | 52734 | 10:76609622 | 52735 | 10:76613357 |
| 52737 | 10:76718459 | 52746 | 10:76886184 | 52808 | 10:78222151 | 52816 | 10:78333529 | 52831 | 10:78834237 |
| 52838 | 10:78993174 | 52847 | 10:79256209 | 52855 | 10:79448706 | 52875 | 10:80033473 | 52893 | 10:80437796 |
| 52905 | 10:80755095 | 52952 | 10:81403359 | 53025 | 10:82946024 | 53076 | 10:84003158 | 53119 | 10:84929419 |
| 53136 | 10:85211692 | 53141 | 10:85308316 | 53146 | 10:85365819 | 53151 | 10:85506776 | 53153 | 10:85532563 |
| 53180 | 10:86322591 | 53190 | 10:86480002 | 53198 | 10:86564397 | 53234 | 10:87380593 | 53240 | 10:87570845 |
| 53303 | 10:88984928 | 53307 | 10:89076810 | 53328 | 10:89477438 | 53338 | 10:89716618 | 53376 | 10:90408131 |
| 53386 | 10:90606603 | 53387 | 10:90610286 | 53410 | 10:90869492 | 53412 | 10:90878413 | 53427 | 10:91270590 |
| 53500 | 10:92937814 | 53506 | 10:93026084 | 53510 | 10:93101284 | 53522 | 10:93436468 | 53527 | 10:93605955 |
| 53581 | 10:94985373 | 53592 | 10:95290678 | 53606 | 10:95624360 | 53629 | 10:96068605 | 53633 | 10:96193408 |
| 53649 | 10:96591095 | 53654 | 10:96786131 | 53655 | 10:96788386 | 53694 | 10:97467089 | 53707 | 10:97772610 |
| 53708 | 10:97775801 | 53709 | 10:97778298 | 53734 | 10:98439866 | 53759 | 10:98730218 | 53782 | 10:99444853 |
| 53785 | 10:99471490 | 53794 | 10:99596036 | 53811 | 10:99982401 | 53873 | 10:10113952 | 53898 | 10:10165041 |
| 53899 | 10:10166055 | 53900 | 10:10167602 | 53902 | 10:10170386 | 53916 | 10:10185170 | 53926 | 10:10206511 |
| 53928 | 10:10208682 | 53933 | 10:10213240 | 53939 | 10:10223950 | 53977 | 10:10315226 | 54020 | 11:779732 |
| 54030 | 11:923580 | 54035 | 11:1009994 | 54047 | 11:1211969 | 54118 | 11:2084360 | 54127 | 11:2256142 |
| 54133 | 11:2359945 | 54181 | 11:2799677 | 54196 | 11:3053638 | 54206 | 11:3153964 | 54238 | 11:3763768 |
| 54328 | 11:5718123 | 54345 | 11:6071587 | 54380 | 11:7143509 | 54398 | 11:7527861 | 54404 | 11:7599092 |
| 54441 | 11:8559109 | 54503 | 11:9819964 | 54512 | 11:10000902 | 54588 | 11:11731827 | 54594 | 11:11844298 |
| 54617 | 11:12454930 | 54653 | 11:13209044 | 54654 | 11:13222009 | 54689 | 11:13914152 | 54696 | 11:14200577 |
| 54727 | 11:15182386 | 54732 | 11:15285660 | 54749 | 11:15700826 | 54770 | 11:16056267 | 54804 | 11:16934584 |
| 54814 | 11:17117515 | 54867 | 11:18770128 | 54928 | 11:20656584 | 54943 | 11:21060821 | 54992 | 11:22018210 |
| 55000 | 11:22303794 | 55012 | 11:22569464 | 55020 | 11:22724476 | 55032 | 11:23083144 | 55068 | 11:23895849 |
| 55087 | 11:24338666 | 55097 | 11:24469097 | 55129 | 11:25273777 | 55140 | 11:25560609 | 55162 | 11:26154618 |
| 55165 | 11:26170393 | 55252 | 11:28239112 | 55304 | 11:29324448 | 55317 | 11:29530060 | 55324 | 11:29724736 |
| 55329 | 11:29759229 | 55355 | 11:30470011 | 55385 | 11:31068333 | 55434 | 11:32501555 | 55466 | 11:33302958 |
| 55503 | 11:34519584 | 55542 | 11:35824586 | 55552 | 11:36039319 | 55557 | 11:36151963 | 55562 | 11:36293996 |
| 55577 | 11:36723444 | 55599 | 11:37215399 | 55600 | 11:37240873 | 55626 | 11:37656828 | 55634 | 11:37841103 |
| 55637 | 11:37898906 | 55672 | 11:39016826 | 55701 | 11:40047215 | 55706 | 11:40222392 | 55716 | 11:40663028 |
| 55751 | 11:41311710 | 55752 | 11:41326914 | 55755 | 11:41328623 | 55758 | 11:41354523 | 55760 | 11:41356073 |
| 55766 | 11:41457760 | 55774 | 11:41843798 | 55796 | 11:42373804 | 55797 | 11:42374333 | 55798 | 11:42374619 |
| 55799 | 11:42374860 | 55800 | 11:42375434 | 55802 | 11:42378608 | 55803 | 11:42395653 | 55804 | 11:42395937 |
| 55808 | 11:42450574 | 55809 | 11:42458071 | 55826 | 11:42952869 | 55828 | 11:42977381 | 55834 | 11:43155397 |
| 55849 | 11:43447636 | 55855 | 11:43656266 | 55867 | 11:44164688 | 55871 | 11:44197049 | 55904 | 11:45053290 |
| 55950 | 11:46193646 | 55973 | 11:46703688 | 55976 | 11:46763745 | 55977 | 11:46763771 | 55978 | 11:46763772 |
| 55979 | 11:46763803 | 55980 | 11:46763967 | 55981 | 11:46764372 | 55982 | 11:46765309 | 55983 | 11:46769693 |
| 56013 | 11:47198478 | 56053 | 11:48210329 | 56122 | 11:48963797 | 56127 | 11:49061005 | 56128 | 11:49080138 |
| 56135 | 11:49179291 | 56138 | 11:49285957 | 56143 | 11:49404465 | 56147 | 11:49422373 | 56152 | 11:49575365 |
| 56153 | 11:49582523 | 56198 | 11:51041604 | 56201 | 11:51160828 | 56205 | 11:51255454 | 56206 | 11:51261576 |
| 56252 | 11:53012268 | 56253 | 11:53022205 | 56257 | 11:53214759 | 56272 | 11:53823211 | 56293 | 11:54411745 |
| 56301 | 11:54607804 | 56302 | 11:54611060 | 56303 | 11:54615185 | 56304 | 11:54619006 | 56305 | 11:54619299 |
| 56306 | 11:54621378 | 56307 | 11:54624272 | 56308 | 11:54625736 | 56309 | 11:54637534 | 56310 | 11:54640117 |
| 56311 | 11:54640249 | 56312 | 11:54644177 | 56313 | 11:54658429 | 56314 | 11:54667025 | 56315 | 11:54672210 |
| 56317 | 11:54687119 | 56318 | 11:54696136 | 56327 | 11:54925623 | 56341 | 11:55201147 | 56345 | 11:55322961 |
| 56384 | 11:56468221 | 56394 | 11:56809772 | 56395 | 11:56811610 | 56396 | 11:56812388 | 56408 | 11:57181716 |
| 56434 | 11:58004110 | 56441 | 11:58172817 | 56451 | 11:58552292 | 56452 | 11:58557598 | 56478 | 11:59155112 |
| 56498 | 11:59762890 | 56519 | 11:60453153 | 56528 | 11:60471884 | 56539 | 11:60491356 | 56609 | 11:62266387 |
| 56611 | 11:62305455 | 56612 | 11:62308757 | 56614 | 11:62379499 | 56617 | 11:62426969 | 56623 | 11:62609754 |
| 56681 | 11:63826838 | 56692 | 11:63976694 | 56707 | 11:64337696 | 56711 | 11:64464647 | 56737 | 11:65172790 |
| 56789 | 11:66422364 | 56887 | 11:68143046 | 56912 | 11:68896819 | 56918 | 11:69065684 | 56919 | 11:69065947 |
| 56920 | 11:69067967 | 56927 | 11:69141312 | 56934 | 11:69198908 | 56936 | 11:69213888 | 56965 | 11:69725037 |
| 56975 | 11:69945179 | 56979 | 11:70105382 | 56989 | 11:70270717 | 56991 | 11:70291428 | 57061 | 11:71167695 |
| 57063 | 11:71169475 | 57064 | 11:71171201 | 57065 | 11:71174044 | 57071 | 11:71187848 | 57083 | 11:71410081 |
| 57092 | 11:71596359 | 57093 | 11:71603815 | 57097 | 11:71735550 | 57118 | 11:72456335 | 57127 | 11:72799464 |
| 57171 | 11:73648343 | 57267 | 11:75692626 | 57288 | 11:76333610 | 57314 | 11:77013992 | 57318 | 11:77067925 |
| 57319 | 11:77077199 | 57320 | 11:77085172 | 57330 | 11:77232878 | 57340 | 11:77445226 | 57341 | 11:77485470 |
| 57396 | 11:78194981 | 57405 | 11:78326150 | 57555 | 11:81830178 | 57571 | 11:82150919 | 57595 | 11:82670314 |
| 57617 | 11:82895182 | 57620 | 11:82943647 | 57646 | 11:83482447 | 57648 | 11:83552676 | 57708 | 11:85258209 |
| 57718 | 11:85436750 | 57725 | 11:85653522 | 57730 | 11:85743559 | 57754 | 11:86206294 | 57755 | 11:86214871 |
| 57814 | 11:87707465 | 57847 | 11:87999059 | 57865 | 11:88032183 | 57884 | 11:88448012 | 57927 | 11:89546657 |
| 57946 | 11:89996070 | 57970 | 11:90503652 | 58023 | 11:91710069 | 58027 | 11:91791559 | 58028 | 11:91792786 |
| 58029 | 11:91796326 | 58030 | 11:91798254 | 58031 | 11:91807646 | 58032 | 11:91809950 | 58033 | 11:91809976 |
| 58042 | 11:92019924 | 58082 | 11:92823026 | 58091 | 11:93153121 | 58092 | 11:93165769 | 58093 | 11:93179602 |
| 58094 | 11:93185994 | 58100 | 11:93232642 | 58105 | 11:93261434 | 58106 | 11:93262306 | 58107 | 11:93262884 |
| 58108 | 11:93263825 | 58109 | 11:93279097 | 58111 | 11:93395136 | 58124 | 11:93858711 | 58143 | 11:94337656 |
| 58163 | 11:94860855 | 58168 | 11:95063641 | 58175 | 11:95296518 | 58268 | 11:97728887 | 58299 | 11:98222359 |
| 58303 | 11:98359510 | 58373 | 11:99755436 | 58492 | 11:10201461 | 58517 | 11:10257709 | 58524 | 11:10265965 |
| 58583 | 11:10338389 | 58596 | 11:10369741 | 58630 | 11:10447397 | 58644 | 11:10468322 | 58654 | 11:10482995 |
| 58656 | 11:10487555 | 58658 | 11:10492751 | 58661 | 11:10497336 | 58663 | 11:10497439 | 58681 | 11:10548246 |
| 58729 | 11:10679315 | 58731 | 11:10694859 | 58752 | 12:625880 | 58780 | 12:1498811 | 58821 | 12:2369495 |
| 58870 | 12:3405573 | 58875 | 12:3491002 | 58894 | 12:4026056 | 58925 | 12:4736478 | 58934 | 12:5016508 |
| 58960 | 12:5810001 | 59000 | 12:7239138 | 59032 | 12:8559085 | 59098 | 12:10166200 | 59115 | 12:10466559 |
| 59148 | 12:11055011 | 59172 | 12:11660529 | 59177 | 12:11743244 | 59237 | 12:13369400 | 59275 | 12:14073643 |
| 59284 | 12:14200873 | 59334 | 12:15275995 | 59404 | 12:16588996 | 59405 | 12:16617595 | 59445 | 12:17717003 |
| 59511 | 12:19127423 | 59512 | 12:19133184 | 59553 | 12:20149260 | 59563 | 12:20389759 | 59565 | 12:20491988 |
| 59566 | 12:20500610 | 59608 | 12:21574455 | 59700 | 12:23970417 | 59718 | 12:24380235 | 59730 | 12:24612900 |
| 59753 | 12:25107687 | 59760 | 12:25333357 | 59778 | 12:25727678 | 59779 | 12:25742060 | 59782 | 12:25755570 |
| 59787 | 12:25862626 | 59816 | 12:26904714 | 59834 | 12:27216744 | 59891 | 12:28914720 | 59952 | 12:30585785 |
| 59960 | 12:30737586 | 59964 | 12:30851151 | 59965 | 12:30851590 | 59978 | 12:31055012 | 60064 | 12:33121234 |
| 60068 | 12:33212754 | 60115 | 12:34706439 | 60158 | 12:35595816 | 60205 | 12:37118156 | 60252 | 12:39318432 |
| 60253 | 12:39337804 | 60254 | 12:39339971 | 60255 | 12:39375252 | 60295 | 12:40436881 | 60299 | 12:40699170 |
| 60310 | 12:41000477 | 60345 | 12:42446263 | 60346 | 12:42529266 | 60384 | 12:44349627 | 60414 | 12:45309570 |
| 60464 | 12:46902819 | 60503 | 12:47894827 | 60512 | 12:48234155 | 60524 | 12:48552141 | 60528 | 12:48621944 |
| 60557 | 12:49347591 | 60588 | 12:50012484 | 60654 | 12:51413041 | 60677 | 12:52057233 | 60712 | 12:52833730 |
| 60713 | 12:52841272 | 60714 | 12:52843772 | 60715 | 12:52859350 | 60717 | 12:52867407 | 60718 | 12:52868520 |
| 60720 | 12:52883259 | 60721 | 12:52888503 | 60736 | 12:53324225 | 60737 | 12:53326935 | 60748 | 12:53513237 |
| 60776 | 12:54070308 | 60778 | 12:54112016 | 60800 | 12:54537212 | 60823 | 12:55077299 | 60834 | 12:55543664 |
| 60864 | 12:56465958 | 60881 | 12:57046134 | 60949 | 12:59148039 | 60950 | 12:59163748 | 60958 | 12:59442298 |
| 61087 | 12:63768535 | 61092 | 12:63878306 | 61108 | 12:64352510 | 61118 | 12:64738377 | 61196 | 12:66786596 |
| 61240 | 12:67928955 | 61247 | 12:68098257 | 61248 | 12:68114447 | 61259 | 12:68270144 | 61275 | 12:68608809 |
| 61278 | 12:68710557 | 61290 | 12:68863135 | 61301 | 12:69019005 | 61309 | 12:69286353 | 61311 | 12:69340936 |
| 61318 | 12:69467691 | 61328 | 12:69553759 | 61332 | 12:69582989 | 61366 | 12:71235763 | 61382 | 12:71600338 |
| 61412 | 12:72958303 | 61413 | 12:72961991 | 61446 | 12:73518731 | 61482 | 12:74197569 | 61497 | 12:74496338 |
| 61522 | 12:75101487 | 61529 | 12:75242221 | 61532 | 12:75277582 | 61537 | 12:75375680 | 61590 | 12:76682892 |
| 61618 | 12:77393088 | 61638 | 12:77847200 | 61645 | 12:77993107 | 61673 | 12:78662420 | 61704 | 12:79284630 |
| 61718 | 12:79542780 | 61728 | 12:79738869 | 61749 | 12:80221064 | 61750 | 12:80223568 | 61751 | 12:80224696 |
| 61755 | 12:80311844 | 61760 | 12:80405755 | 61762 | 12:80523810 | 61818 | 12:81778398 | 61836 | 12:82156928 |
| 61850 | 12:82530033 | 61865 | 12:82796759 | 61888 | 12:83319494 | 61922 | 12:84042281 | 61956 | 12:84892891 |
| 61968 | 12:85033607 | 61980 | 12:85234076 | 61998 | 12:85446719 | 62009 | 12:85641586 | 62011 | 12:85752928 |
| 62060 | 12:86779904 | 62103 | 13:648927 | 62135 | 13:1384950 | 62142 | 13:1583266 | 62183 | 13:2254372 |
| 62213 | 13:2708064 | 62260 | 13:3861084 | 62336 | 13:5664259 | 62337 | 13:5666787 | 62361 | 13:6275220 |
| 62366 | 13:6361450 | 62408 | 13:7483031 | 62413 | 13:7556779 | 62434 | 13:8333741 | 62438 | 13:8415584 |
| 62443 | 13:8529126 | 62444 | 13:8533157 | 62445 | 13:8537080 | 62446 | 13:8542772 | 62450 | 13:8682255 |
| 62451 | 13:8682878 | 62452 | 13:8683708 | 62453 | 13:8685414 | 62454 | 13:8686278 | 62524 | 13:10400290 |
| 62526 | 13:10426820 | 62537 | 13:10575199 | 62553 | 13:11047542 | 62573 | 13:12140250 | 62613 | 13:13240348 |
| 62654 | 13:14380630 | 62665 | 13:14649538 | 62669 | 13:14768979 | 62675 | 13:14878218 | 62690 | 13:15298554 |
| 62693 | 13:15509230 | 62707 | 13:15907128 | 62845 | 13:18649928 | 62849 | 13:18704268 | 62880 | 13:18741567 |
| 62886 | 13:18795212 | 62893 | 13:18926634 | 62904 | 13:19113938 | 62927 | 13:19611692 | 62953 | 13:19991782 |
| 62955 | 13:20085636 | 62962 | 13:20299025 | 62985 | 13:20633696 | 62991 | 13:20728403 | 62999 | 13:21031730 |
| 63014 | 13:21160488 | 63016 | 13:21199075 | 63039 | 13:21675803 | 63072 | 13:22491633 | 63080 | 13:22645333 |
| 63142 | 13:24652712 | 63154 | 13:24964331 | 63170 | 13:25313389 | 63233 | 13:26864651 | 63296 | 13:28352469 |
| 63310 | 13:28677951 | 63311 | 13:28716131 | 63313 | 13:28719397 | 63314 | 13:28721486 | 63315 | 13:28726672 |
| 63328 | 13:29164280 | 63331 | 13:29203677 | 63409 | 13:30734642 | 63413 | 13:30835419 | 63417 | 13:30928296 |
| 63433 | 13:31170980 | 63479 | 13:32132576 | 63480 | 13:32133958 | 63481 | 13:32143886 | 63482 | 13:32152278 |
| 63494 | 13:32274709 | 63506 | 13:32489566 | 63521 | 13:32651702 | 63522 | 13:32653621 | 63523 | 13:32654103 |
| 63528 | 13:32846791 | 63529 | 13:32846825 | 63530 | 13:32847172 | 63531 | 13:32847400 | 63542 | 13:33084958 |
| 63543 | 13:33086348 | 63572 | 13:33880293 | 63599 | 13:34800113 | 63619 | 13:35179138 | 63622 | 13:35197458 |
| 63766 | 13:38636130 | 63767 | 13:38662619 | 63768 | 13:38676202 | 63769 | 13:38680784 | 63775 | 13:38810147 |
| 63781 | 13:38839160 | 63782 | 13:38893419 | 63786 | 13:38949069 | 63796 | 13:39090329 | 63800 | 13:39121053 |
| 63832 | 13:39696126 | 63852 | 13:40100334 | 63860 | 13:40220102 | 63870 | 13:40453192 | 63872 | 13:40473511 |
| 63873 | 13:40486594 | 63874 | 13:40486661 | 63940 | 13:42071486 | 63972 | 13:42587452 | 63982 | 13:42606861 |
| 64003 | 13:42825481 | 64024 | 13:43516130 | 64032 | 13:43689434 | 64033 | 13:43726105 | 64039 | 13:43774352 |
| 64059 | 13:44057830 | 64060 | 13:44115215 | 64105 | 13:44933952 | 64122 | 13:44991547 | 64157 | 13:45403608 |
| 64176 | 13:45936548 | 64210 | 13:46658312 | 64216 | 13:46758989 | 64245 | 13:47366925 | 64250 | 13:47472066 |
| 64254 | 13:47495500 | 64264 | 13:47827146 | 64284 | 13:48238821 | 64285 | 13:48240137 | 64286 | 13:48240290 |
| 64287 | 13:48240322 | 64288 | 13:48240351 | 64289 | 13:48241221 | 64290 | 13:48241373 | 64291 | 13:48241390 |
| 64292 | 13:48243603 | 64293 | 13:48245013 | 64294 | 13:48245053 | 64296 | 13:48269729 | 64297 | 13:48274446 |
| 64318 | 13:49199089 | 64319 | 13:49199105 | 64320 | 13:49200231 | 64419 | 13:52063310 | 64429 | 13:52466434 |
| 64496 | 13:53368735 | 64499 | 13:53503066 | 64516 | 13:54246719 | 64522 | 13:54512037 | 64533 | 13:54732415 |
| 64593 | 13:56026065 | 64649 | 13:57529999 | 64650 | 13:57541123 | 64669 | 13:58019158 | 64698 | 13:58633062 |
| 64708 | 13:58899200 | 64721 | 13:59016374 | 64732 | 13:59248087 | 64735 | 13:59289762 | 64766 | 13:60057972 |
| 64776 | 13:60212933 | 64798 | 13:60664451 | 64807 | 13:60980294 | 64822 | 13:61522788 | 64846 | 13:62118304 |
| 64852 | 13:62277677 | 64888 | 13:62739693 | 64889 | 13:62752247 | 64893 | 13:62800301 | 64899 | 13:62843052 |
| 64946 | 13:63972761 | 64967 | 13:64523897 | 64988 | 13:65061778 | 65005 | 13:65480528 | 65011 | 13:65604335 |
| 65014 | 13:65656406 | 65021 | 13:65714427 | 65023 | 13:65715682 | 65033 | 13:65806803 | 65052 | 13:66249739 |
| 65070 | 13:66636049 | 65076 | 13:66754479 | 65077 | 13:66811790 | 65082 | 13:66930495 | 65099 | 13:67376538 |
| 65112 | 13:67740112 | 65114 | 13:67768319 | 65118 | 13:67910634 | 65141 | 13:68545291 | 65177 | 13:69533712 |
| 65193 | 13:69946188 | 65219 | 13:70559101 | 65254 | 13:71281225 | 65255 | 13:71297993 | 65264 | 13:71463110 |
| 65285 | 13:71937940 | 65291 | 13:72156689 | 65345 | 13:73285435 | 65394 | 13:74233601 | 65400 | 13:74368669 |
| 65402 | 13:74389287 | 65435 | 13:74875799 | 65458 | 13:75539335 | 65459 | 13:75546891 | 65460 | 13:75550544 |
| 65475 | 13:75773530 | 65490 | 13:76048819 | 65495 | 13:76206814 | 65497 | 13:76256932 | 65513 | 13:76540633 |
| 65533 | 13:76870141 | 65610 | 13:78594569 | 65632 | 13:78943793 | 65642 | 13:79095868 | 65658 | 13:79307341 |
| 65667 | 13:79411595 | 65668 | 13:79427565 | 65687 | 13:79896093 | 65693 | 13:80009218 | 65699 | 13:80110062 |
| 65700 | 13:80110693 | 65701 | 13:80112377 | 65702 | 13:80112537 | 65703 | 13:80112873 | 65704 | 13:80113035 |
| 65705 | 13:80113195 | 65706 | 13:80119481 | 65707 | 13:80120615 | 65708 | 13:80121232 | 65709 | 13:80121378 |
| 65710 | 13:80121492 | 65711 | 13:80121527 | 65712 | 13:80122117 | 65713 | 13:80123053 | 65714 | 13:80123496 |
| 65715 | 13:80123540 | 65716 | 13:80128525 | 65717 | 13:80128577 | 65718 | 13:80128592 | 65719 | 13:80128724 |
| 65720 | 13:80133414 | 65726 | 13:80284611 | 65730 | 13:80353010 | 65731 | 13:80354577 | 65741 | 13:80564883 |
| 65792 | 13:81650045 | 65813 | 13:82057542 | 65814 | 13:82060563 | 65847 | 13:82692047 | 65937 | 14:1748354 |
| 65939 | 14:1780705 | 65960 | 14:2169492 | 65962 | 14:2196270 | 65979 | 14:2821082 | 65999 | 14:3180090 |
| 66000 | 14:3180092 | 66001 | 14:3184037 | 66003 | 14:3188747 | 66031 | 14:3341005 | 66057 | 14:3777934 |
| 66059 | 14:3807311 | 66065 | 14:3937653 | 66096 | 14:4438274 | 66107 | 14:4826114 | 66109 | 14:4845905 |
| 66112 | 14:4900819 | 66114 | 14:4924180 | 66123 | 14:5079999 | 66141 | 14:5424566 | 66143 | 14:5442736 |
| 66144 | 14:5442915 | 66155 | 14:5642922 | 66161 | 14:5745989 | 66162 | 14:5746008 | 66163 | 14:5746079 |
| 66164 | 14:5746088 | 66167 | 14:5767279 | 66168 | 14:5767316 | 66182 | 14:6051710 | 66222 | 14:6580835 |
| 66251 | 14:7127015 | 66255 | 14:7190153 | 66260 | 14:7315224 | 66267 | 14:7446316 | 66296 | 14:7973769 |
| 66298 | 14:7981735 | 66299 | 14:7984017 | 66301 | 14:7993501 | 66303 | 14:8000324 | 66306 | 14:8006167 |
| 66307 | 14:8008471 | 66309 | 14:8029440 | 66324 | 14:8315882 | 66339 | 14:8555196 | 66342 | 14:8578594 |
| 66401 | 14:9370788 | 66424 | 14:9883880 | 66445 | 14:10330270 | 66487 | 14:11125973 | 66519 | 14:11675980 |
| 66520 | 14:11687647 | 66521 | 14:11700111 | 66536 | 14:12017220 | 66572 | 14:12683380 | 66573 | 14:12683869 |
| 66575 | 14:12685152 | 66576 | 14:12685153 | 66580 | 14:12718898 | 66581 | 14:12726976 | 66582 | 14:12726981 |
| 66642 | 14:14433818 | 66674 | 14:15281409 | 66683 | 14:15476637 | 66707 | 14:15871072 | 66740 | 14:16464011 |
| 66800 | 14:17774684 | 66848 | 14:18880176 | 66911 | 14:20615962 | 66917 | 14:20673221 | 66920 | 14:20723405 |
| 66921 | 14:20728338 | 66939 | 14:21119658 | 66940 | 14:21124340 | 66973 | 14:21853648 | 67075 | 14:22718406 |
| 67158 | 14:23199725 | 67159 | 14:23200416 | 67160 | 14:23200440 | 67161 | 14:23202111 | 67162 | 14:23202373 |
| 67163 | 14:23202574 | 67164 | 14:23202642 | 67165 | 14:23202665 | 67166 | 14:23205217 | 67167 | 14:23206165 |
| 67168 | 14:23209278 | 67169 | 14:23210217 | 67170 | 14:23211671 | 67171 | 14:23212276 | 67172 | 14:23212424 |
| 67174 | 14:23216603 | 67176 | 14:23216967 | 67177 | 14:23217298 | 67178 | 14:23217676 | 67179 | 14:23217682 |
| 67182 | 14:23217984 | 67184 | 14:23219378 | 67185 | 14:23220306 | 67186 | 14:23221060 | 67187 | 14:23221105 |
| 67188 | 14:23221660 | 67190 | 14:23223448 | 67192 | 14:23232920 | 67293 | 14:23296927 | 67353 | 14:23590382 |
| 67532 | 14:25538462 | 67551 | 14:25555299 | 67553 | 14:25557838 | 67562 | 14:25875465 | 67572 | 14:26025356 |
| 67592 | 14:26183095 | 67615 | 14:26313111 | 67631 | 14:26716130 | 67638 | 14:26937569 | 67647 | 14:27145363 |
| 67659 | 14:27578987 | 67666 | 14:27804049 | 67669 | 14:27925661 | 67680 | 14:28283881 | 67684 | 14:28475888 |
| 67701 | 14:28805553 | 67713 | 14:29204640 | 67728 | 14:29506987 | 67737 | 14:29651348 | 67751 | 14:30011410 |
| 67787 | 14:30531165 | 67806 | 14:31151661 | 67810 | 14:31229071 | 67812 | 14:31276668 | 67813 | 14:31348707 |
| 67839 | 14:32042320 | 67840 | 14:32080892 | 67847 | 14:32295836 | 67848 | 14:32298088 | 67849 | 14:32298121 |
| 67850 | 14:32298404 | 67851 | 14:32298532 | 67852 | 14:32298550 | 67853 | 14:32298592 | 67854 | 14:32298623 |
| 67855 | 14:32298715 | 67856 | 14:32298807 | 67857 | 14:32298889 | 67858 | 14:32298929 | 67859 | 14:32298949 |
| 67860 | 14:32298983 | 67861 | 14:32301242 | 67862 | 14:32301629 | 67863 | 14:32301804 | 67880 | 14:32768091 |
| 67902 | 14:33283287 | 67914 | 14:33481134 | 67945 | 14:34107991 | 67963 | 14:34616797 | 67966 | 14:34703575 |
| 67968 | 14:34726901 | 68040 | 14:36456652 | 68066 | 14:37335063 | 68131 | 14:39204823 | 68180 | 14:40564678 |
| 68185 | 14:40761575 | 68199 | 14:41245865 | 68207 | 14:41477789 | 68211 | 14:41608564 | 68246 | 14:42371257 |
| 68252 | 14:42499551 | 68275 | 14:43217965 | 68314 | 14:44597633 | 68367 | 14:45598918 | 68386 | 14:45953548 |
| 68393 | 14:46043440 | 68412 | 14:46302755 | 68430 | 14:46746014 | 68435 | 14:46846924 | 68463 | 14:47562082 |
| 68479 | 14:48154116 | 68495 | 14:48422440 | 68496 | 14:48424765 | 68507 | 14:48722429 | 68578 | 14:50964252 |
| 68611 | 14:51741282 | 68659 | 14:53067901 | 68716 | 14:54352030 | 68717 | 14:54359397 | 68727 | 14:54470262 |
| 68733 | 14:54629600 | 68785 | 14:56030241 | 68869 | 14:58169542 | 68876 | 14:58268988 | 68879 | 14:58347367 |
| 68898 | 14:58795728 | 68902 | 14:58882362 | 68914 | 14:59072940 | 68931 | 14:59422421 | 68945 | 14:59890123 |
| 68960 | 14:60186718 | 68998 | 14:61242862 | 69030 | 14:61805601 | 69069 | 14:62451453 | 69075 | 14:62584372 |
| 69079 | 14:62700380 | 69092 | 14:62957699 | 69106 | 14:63240134 | 69117 | 14:63473862 | 69160 | 14:64610567 |
| 69174 | 14:65146328 | 69215 | 14:66160998 | 69219 | 14:66259689 | 69228 | 14:66384994 | 69232 | 14:66419660 |
| 69326 | 14:68515393 | 69354 | 14:69351733 | 69370 | 14:69913790 | 69382 | 14:70082152 | 69391 | 14:70201845 |
| 69400 | 14:70384514 | 69408 | 14:70595169 | 69415 | 14:70881552 | 69416 | 14:70890232 | 69427 | 14:71123353 |
| 69443 | 14:71687136 | 69472 | 14:72410713 | 69494 | 14:72897923 | 69506 | 14:73212493 | 69515 | 14:73427877 |
| 69517 | 14:73468390 | 69534 | 14:73985016 | 69570 | 14:75029436 | 69571 | 14:75034802 | 69572 | 14:75035607 |
| 69573 | 14:75035827 | 69574 | 14:75035857 | 69575 | 14:75035923 | 69608 | 14:76029716 | 69631 | 14:76258289 |
| 69633 | 14:76268094 | 69680 | 14:77464768 | 69686 | 14:77542458 | 69690 | 14:77708662 | 69700 | 14:77954720 |
| 69736 | 14:78637209 | 69737 | 14:78644200 | 69742 | 14:78699024 | 69750 | 14:78929919 | 69762 | 14:79335372 |
| 69767 | 14:79486373 | 69783 | 14:79820967 | 69790 | 14:79974645 | 69805 | 14:80236030 | 69819 | 14:80591048 |
| 69864 | 14:81465207 | 69901 | 14:82003511 | 70000 | 15:2555400 | 70037 | 15:3651481 | 70043 | 15:3796804 |
| 70044 | 15:3859257 | 70045 | 15:3861053 | 70046 | 15:3861177 | 70048 | 15:3872949 | 70049 | 15:3874148 |
| 70050 | 15:3874413 | 70051 | 15:3875123 | 70052 | 15:3875291 | 70053 | 15:3875480 | 70054 | 15:3875800 |
| 70055 | 15:3876161 | 70056 | 15:3876211 | 70057 | 15:3876259 | 70058 | 15:3884992 | 70059 | 15:3885455 |
| 70060 | 15:3888242 | 70061 | 15:3888270 | 70070 | 15:4006347 | 70080 | 15:4274305 | 70107 | 15:4817619 |
| 70108 | 15:4821833 | 70109 | 15:4827554 | 70110 | 15:4850527 | 70111 | 15:4855973 | 70113 | 15:4859691 |
| 70114 | 15:4874530 | 70132 | 15:5345428 | 70133 | 15:5345893 | 70134 | 15:5346513 | 70135 | 15:5346544 |
| 70136 | 15:5347645 | 70137 | 15:5348421 | 70138 | 15:5348505 | 70139 | 15:5348800 | 70140 | 15:5348852 |
| 70141 | 15:5348875 | 70142 | 15:5349143 | 70143 | 15:5349849 | 70144 | 15:5350256 | 70145 | 15:5350323 |
| 70146 | 15:5350355 | 70147 | 15:5353059 | 70148 | 15:5353099 | 70149 | 15:5355811 | 70150 | 15:5355909 |
| 70151 | 15:5356157 | 70152 | 15:5356186 | 70153 | 15:5356365 | 70154 | 15:5356372 | 70155 | 15:5357215 |
| 70156 | 15:5358118 | 70157 | 15:5358230 | 70158 | 15:5358668 | 70159 | 15:5359593 | 70160 | 15:5359865 |
| 70161 | 15:5359868 | 70162 | 15:5361660 | 70163 | 15:5361661 | 70164 | 15:5365576 | 70165 | 15:5380375 |
| 70166 | 15:5381250 | 70167 | 15:5382096 | 70168 | 15:5383165 | 70169 | 15:5383541 | 70170 | 15:5384183 |
| 70171 | 15:5384848 | 70172 | 15:5384963 | 70173 | 15:5384997 | 70174 | 15:5385062 | 70175 | 15:5385270 |
| 70176 | 15:5385290 | 70177 | 15:5385312 | 70178 | 15:5385429 | 70179 | 15:5386627 | 70180 | 15:5386633 |
| 70181 | 15:5386655 | 70182 | 15:5386721 | 70183 | 15:5387773 | 70184 | 15:5387850 | 70185 | 15:5388162 |
| 70186 | 15:5388165 | 70187 | 15:5388635 | 70188 | 15:5388661 | 70189 | 15:5388748 | 70190 | 15:5389424 |
| 70191 | 15:5389588 | 70192 | 15:5389616 | 70193 | 15:5389859 | 70194 | 15:5389978 | 70195 | 15:5390296 |
| 70196 | 15:5390320 | 70197 | 15:5390450 | 70198 | 15:5390553 | 70199 | 15:5390863 | 70200 | 15:5390921 |
| 70201 | 15:5391192 | 70202 | 15:5391298 | 70203 | 15:5391508 | 70204 | 15:5391682 | 70205 | 15:5391799 |
| 70206 | 15:5392452 | 70207 | 15:5392859 | 70208 | 15:5392923 | 70209 | 15:5393194 | 70210 | 15:5393401 |
| 70211 | 15:5393671 | 70212 | 15:5393753 | 70213 | 15:5394283 | 70214 | 15:5395547 | 70215 | 15:5395610 |
| 70216 | 15:5395743 | 70217 | 15:5395751 | 70218 | 15:5395855 | 70219 | 15:5395947 | 70220 | 15:5398109 |
| 70221 | 15:5398341 | 70222 | 15:5398346 | 70223 | 15:5398513 | 70224 | 15:5398559 | 70225 | 15:5400626 |
| 70226 | 15:5401120 | 70227 | 15:5401525 | 70228 | 15:5401536 | 70229 | 15:5401568 | 70230 | 15:5401820 |
| 70231 | 15:5401853 | 70232 | 15:5404746 | 70233 | 15:5404825 | 70234 | 15:5405498 | 70235 | 15:5405505 |
| 70236 | 15:5406067 | 70237 | 15:5406105 | 70238 | 15:5406213 | 70239 | 15:5406470 | 70240 | 15:5406961 |
| 70241 | 15:5407075 | 70242 | 15:5407142 | 70243 | 15:5407235 | 70244 | 15:5407350 | 70245 | 15:5407539 |
| 70246 | 15:5407760 | 70247 | 15:5407841 | 70248 | 15:5407895 | 70249 | 15:5408434 | 70250 | 15:5408552 |
| 70251 | 15:5408640 | 70252 | 15:5408900 | 70253 | 15:5409527 | 70254 | 15:5409532 | 70255 | 15:5409637 |
| 70256 | 15:5409728 | 70257 | 15:5409914 | 70258 | 15:5410082 | 70259 | 15:5410166 | 70260 | 15:5410173 |
| 70261 | 15:5410337 | 70262 | 15:5410342 | 70263 | 15:5410429 | 70264 | 15:5410813 | 70265 | 15:5410848 |
| 70266 | 15:5412771 | 70267 | 15:5412796 | 70268 | 15:5412835 | 70269 | 15:5412869 | 70270 | 15:5413090 |
| 70271 | 15:5413111 | 70272 | 15:5413650 | 70273 | 15:5415034 | 70274 | 15:5415477 | 70275 | 15:5416298 |
| 70276 | 15:5416324 | 70277 | 15:5417129 | 70278 | 15:5417450 | 70279 | 15:5417663 | 70280 | 15:5418542 |
| 70284 | 15:5503948 | 70303 | 15:6004286 | 70319 | 15:6435496 | 70337 | 15:7003211 | 70338 | 15:7008452 |
| 70340 | 15:7019231 | 70341 | 15:7029657 | 70342 | 15:7033643 | 70348 | 15:7265163 | 70349 | 15:7265165 |
| 70350 | 15:7265341 | 70351 | 15:7265425 | 70352 | 15:7265838 | 70353 | 15:7270627 | 70354 | 15:7270650 |
| 70355 | 15:7272155 | 70356 | 15:7272347 | 70357 | 15:7272651 | 70358 | 15:7275537 | 70359 | 15:7275789 |
| 70360 | 15:7275947 | 70361 | 15:7276254 | 70362 | 15:7277640 | 70363 | 15:7277661 | 70364 | 15:7278217 |
| 70411 | 15:8492046 | 70427 | 15:9014277 | 70429 | 15:9031126 | 70432 | 15:9102618 | 70438 | 15:9169770 |
| 70439 | 15:9233443 | 70471 | 15:10068806 | 70472 | 15:10075553 | 70474 | 15:10079990 | 70475 | 15:10080765 |
| 70476 | 15:10081644 | 70511 | 15:11396612 | 70557 | 15:13243732 | 70596 | 15:14363124 | 70609 | 15:14575841 |
| 70610 | 15:14594338 | 70629 | 15:15205635 | 70630 | 15:15239992 | 70707 | 15:16376589 | 70708 | 15:16439066 |
| 70735 | 15:17085252 | 70738 | 15:17150531 | 70745 | 15:17184652 | 70765 | 15:17609663 | 70797 | 15:18443747 |
| 70805 | 15:18738117 | 70819 | 15:19107499 | 70839 | 15:19655124 | 70859 | 15:20139691 | 70869 | 15:20440646 |
| 70890 | 15:20723501 | 70945 | 15:22143335 | 70975 | 15:22778474 | 70976 | 15:22778563 | 70977 | 15:22779768 |
| 70984 | 15:22928839 | 70990 | 15:23127718 | 70993 | 15:23186412 | 71073 | 15:24244322 | 71098 | 15:24733535 |
| 71100 | 15:24758009 | 71125 | 15:25277078 | 71134 | 15:25472864 | 71147 | 15:25791312 | 71158 | 15:25955785 |
| 71179 | 15:26356076 | 71196 | 15:26746546 | 71208 | 15:27116571 | 71241 | 15:27939885 | 71250 | 15:28057977 |
| 71255 | 15:28119260 | 71281 | 15:28579106 | 71361 | 15:30204684 | 71411 | 15:31423842 | 71480 | 15:32908026 |
| 71484 | 15:32949060 | 71520 | 15:33787224 | 71547 | 15:34234555 | 71557 | 15:34409813 | 71575 | 15:34768501 |
| 71595 | 15:35125952 | 71629 | 15:36004891 | 71630 | 15:36008307 | 71631 | 15:36026492 | 71648 | 15:36401889 |
| 71659 | 15:36632711 | 71691 | 15:37336930 | 71694 | 15:37400581 | 71695 | 15:37400733 | 71696 | 15:37402318 |
| 71697 | 15:37402363 | 71698 | 15:37402682 | 71699 | 15:37403163 | 71700 | 15:37405162 | 71701 | 15:37406604 |
| 71702 | 15:37408508 | 71726 | 15:38124244 | 71729 | 15:38238774 | 71747 | 15:38627437 | 71754 | 15:38756627 |
| 71765 | 15:39027306 | 71774 | 15:39310122 | 71779 | 15:39360198 | 71788 | 15:39612413 | 71831 | 15:40620626 |
| 71848 | 15:41008572 | 71872 | 15:41577758 | 71873 | 15:41586379 | 71894 | 15:41925488 | 71937 | 15:42825209 |
| 71945 | 15:42913923 | 71990 | 15:43478979 | 72010 | 15:43778894 | 72015 | 15:43842403 | 72025 | 15:43940904 |
| 72031 | 15:44171870 | 72055 | 15:44625080 | 72119 | 15:46158689 | 72132 | 15:46492762 | 72164 | 15:46744389 |
| 72165 | 15:46777715 | 72167 | 15:46785999 | 72168 | 15:46790581 | 72169 | 15:46804156 | 72170 | 15:46852906 |
| 72171 | 15:46854891 | 72175 | 15:46873315 | 72219 | 15:47951518 | 72223 | 15:48016378 | 72242 | 15:48211767 |
| 72259 | 15:48584212 | 72288 | 15:49705651 | 72400 | 15:52067358 | 72432 | 15:52906987 | 72450 | 15:53308730 |
| 72455 | 15:53312462 | 72514 | 15:54171179 | 72571 | 15:55496596 | 72580 | 15:55679447 | 72584 | 15:55794756 |
| 72629 | 15:56617358 | 72704 | 15:57721772 | 72710 | 15:57822336 | 72753 | 15:58596845 | 72786 | 15:59497090 |
| 72810 | 15:60299723 | 72821 | 15:60544990 | 72828 | 15:60665356 | 72838 | 15:61116189 | 72841 | 15:61144336 |
| 72844 | 15:61187610 | 72847 | 15:61230749 | 72848 | 15:61235200 | 72883 | 15:61807140 | 72897 | 15:62087105 |
| 72904 | 15:62399301 | 72913 | 15:62562520 | 72945 | 15:63169519 | 72956 | 15:63484094 | 72978 | 15:63866432 |
| 72980 | 15:63956384 | 72984 | 15:63969563 | 73010 | 15:64570035 | 73011 | 15:64573599 | 73012 | 15:64581068 |
| 73013 | 15:64581832 | 73014 | 15:64581860 | 73015 | 15:64587577 | 73016 | 15:64587694 | 73017 | 15:64587726 |
| 73018 | 15:64587727 | 73019 | 15:64587894 | 73032 | 15:64805974 | 73034 | 15:64822114 | 73036 | 15:64828957 |
| 73037 | 15:64829763 | 73055 | 15:65133696 | 73057 | 15:65176452 | 73068 | 15:65338322 | 73110 | 15:66198995 |
| 73133 | 15:66626921 | 73136 | 15:66654107 | 73137 | 15:66659219 | 73157 | 15:67252520 | 73183 | 15:67982185 |
| 73232 | 15:69401513 | 73297 | 15:71439330 | 73316 | 15:71924774 | 73318 | 15:71954992 | 73320 | 15:71975292 |
| 73324 | 15:72049215 | 73432 | 15:74395188 | 73433 | 15:74409395 | 73453 | 15:74803075 | 73462 | 15:75064377 |
| 73463 | 15:75065891 | 73464 | 15:75071311 | 73469 | 15:75183095 | 73471 | 15:75207042 | 73478 | 15:75433003 |
| 73581 | 15:77653525 | 73585 | 15:77702400 | 73588 | 15:77785862 | 73593 | 15:77886217 | 73647 | 15:78764970 |
| 73658 | 15:79089495 | 73691 | 15:80200231 | 73697 | 15:80308461 | 73708 | 15:80425465 | 73780 | 15:81982872 |
| 73833 | 15:83017252 | 73857 | 15:83501029 | 73858 | 15:83507518 | 73859 | 15:83514913 | 73860 | 15:83519848 |
| 73861 | 15:83524365 | 73863 | 15:83529608 | 73885 | 15:84090905 | 73971 | 16:1756551 | 74018 | 16:2807132 |
| 74022 | 16:2880813 | 74046 | 16:3278886 | 74067 | 16:3645905 | 74074 | 16:3832226 | 74078 | 16:3983617 |
| 74085 | 16:4041201 | 74113 | 16:4472306 | 74116 | 16:4488724 | 74134 | 16:5011968 | 74222 | 16:7875660 |
| 74227 | 16:8061836 | 74247 | 16:8833166 | 74264 | 16:9480615 | 74299 | 16:10761697 | 74367 | 16:12564264 |
| 74370 | 16:12610513 | 74376 | 16:12712264 | 74380 | 16:12809176 | 74416 | 16:13500639 | 74493 | 16:15596092 |
| 74509 | 16:16114051 | 74543 | 16:17341478 | 74577 | 16:18497037 | 74680 | 16:21073857 | 74697 | 16:21482215 |
| 74705 | 16:21676658 | 74716 | 16:22006522 | 74717 | 16:22052486 | 74721 | 16:22158028 | 74731 | 16:22445521 |
| 74735 | 16:22522151 | 74736 | 16:22527368 | 74784 | 16:23721556 | 74795 | 16:23949095 | 74810 | 16:24332233 |
| 74873 | 16:25194415 | 74886 | 16:25230576 | 74895 | 16:25259731 | 74896 | 16:25267299 | 74912 | 16:25869035 |
| 74914 | 16:25919271 | 74922 | 16:26068625 | 74962 | 16:26927299 | 75005 | 16:28019627 | 75093 | 16:30731403 |
| 75115 | 16:31224822 | 75116 | 16:31245895 | 75135 | 16:31715125 | 75141 | 16:31812156 | 75192 | 16:33108294 |
| 75193 | 16:33145311 | 75196 | 16:33256074 | 75208 | 16:33505042 | 75221 | 16:33889094 | 75227 | 16:34042467 |
| 75228 | 16:34043548 | 75232 | 16:34106781 | 75248 | 16:34589155 | 75329 | 16:36525688 | 75352 | 16:37160322 |
| 75435 | 16:39120749 | 75447 | 16:39536783 | 75474 | 16:40415228 | 75484 | 16:40686277 | 75519 | 16:41883664 |
| 75545 | 16:42004893 | 75548 | 16:42055479 | 75562 | 16:42802530 | 75565 | 16:42843655 | 75640 | 16:46097209 |
| 75644 | 16:46156095 | 75677 | 16:46962612 | 75715 | 16:47752936 | 75724 | 16:48024037 | 75725 | 16:48025956 |
| 75726 | 16:48033049 | 75727 | 16:48034263 | 75729 | 16:48067867 | 75734 | 16:48186793 | 75735 | 16:48187331 |
| 75736 | 16:48187489 | 75737 | 16:48223403 | 75738 | 16:48230117 | 75739 | 16:48232464 | 75751 | 16:48528203 |
| 75754 | 16:48566545 | 75755 | 16:48591914 | 75771 | 16:48864424 | 75776 | 16:49014011 | 75786 | 16:49264493 |
| 75852 | 16:51207748 | 75916 | 16:52579557 | 76051 | 16:56128576 | 76058 | 16:56235648 | 76083 | 16:56653049 |
| 76110 | 16:57538081 | 76150 | 16:58399171 | 76164 | 16:58621735 | 76178 | 16:58963242 | 76182 | 16:59073877 |
| 76184 | 16:59075986 | 76190 | 16:59245993 | 76192 | 16:59265813 | 76193 | 16:59266170 | 76200 | 16:59328729 |
| 76205 | 16:59411723 | 76226 | 16:60040160 | 76243 | 16:60336822 | 76259 | 16:60792834 | 76289 | 16:61435887 |
| 76345 | 16:62739421 | 76365 | 16:63159271 | 76368 | 16:63219362 | 76383 | 16:63498892 | 76385 | 16:63554652 |
| 76398 | 16:63842771 | 76400 | 16:63858162 | 76411 | 16:64136818 | 76424 | 16:64408574 | 76435 | 16:64563350 |
| 76440 | 16:64632519 | 76442 | 16:64637819 | 76443 | 16:64637824 | 76444 | 16:64637826 | 76453 | 16:64811600 |
| 76524 | 16:66273725 | 76529 | 16:66428578 | 76542 | 16:66625242 | 76552 | 16:66834448 | 76585 | 16:67467822 |
| 76587 | 16:67495360 | 76590 | 16:67506416 | 76593 | 16:67552727 | 76603 | 16:67685441 | 76635 | 16:68623796 |
| 76640 | 16:68718917 | 76642 | 16:68746747 | 76646 | 16:68890172 | 76655 | 16:69079413 | 76691 | 16:69777086 |
| 76693 | 16:69813037 | 76701 | 16:70045391 | 76717 | 16:70339833 | 76729 | 16:70576773 | 76748 | 16:70946593 |
| 76774 | 16:71760780 | 76788 | 16:72102560 | 76825 | 16:72662418 | 76830 | 16:72687591 | 76842 | 16:72970330 |
| 76861 | 16:73473194 | 76864 | 16:73513959 | 76895 | 16:73873564 | 76951 | 16:75067479 | 76972 | 16:75350380 |
| 76990 | 16:75714163 | 77032 | 16:76538771 | 77064 | 16:77153817 | 77099 | 16:77965851 | 77102 | 16:78019437 |
| 77132 | 16:78710736 | 77152 | 16:78992583 | 77166 | 16:79102626 | 77238 | 16:80715002 | 77241 | 16:80886197 |
| 77246 | 17:111106 | 77250 | 17:171222 | 77251 | 17:172400 | 77253 | 17:305126 | 77262 | 17:486383 |
| 77374 | 17:3335273 | 77379 | 17:3441488 | 77422 | 17:4260319 | 77443 | 17:4584704 | 77446 | 17:4621398 |
| 77452 | 17:4717447 | 77469 | 17:5097696 | 77470 | 17:5097857 | 77489 | 17:5622055 | 77510 | 17:6077823 |
| 77557 | 17:6872378 | 77579 | 17:7317589 | 77580 | 17:7338578 | 77582 | 17:7342145 | 77586 | 17:7478372 |
| 77606 | 17:7876357 | 77608 | 17:7883935 | 77619 | 17:8132536 | 77638 | 17:8600944 | 77642 | 17:8668787 |
| 77704 | 17:10109146 | 77716 | 17:10263523 | 77725 | 17:10597515 | 77742 | 17:11173860 | 77747 | 17:11293045 |
| 77753 | 17:11395090 | 77762 | 17:11714108 | 77763 | 17:11715058 | 77770 | 17:11817054 | 77787 | 17:12195499 |
| 77788 | 17:12195568 | 77789 | 17:12195662 | 77790 | 17:12196276 | 77791 | 17:12196708 | 77805 | 17:12486159 |
| 77808 | 17:12628635 | 77906 | 17:14816935 | 77909 | 17:14863506 | 77931 | 17:15328888 | 77949 | 17:15663502 |
| 77958 | 17:15910083 | 77975 | 17:16368625 | 78009 | 17:17274244 | 78010 | 17:17279868 | 78011 | 17:17280788 |
| 78012 | 17:17280990 | 78013 | 17:17281225 | 78024 | 17:17411908 | 78039 | 17:17775173 | 78082 | 17:18818243 |
| 78086 | 17:18869685 | 78105 | 17:19223651 | 78106 | 17:19245275 | 78109 | 17:19292348 | 78110 | 17:19295355 |
| 78111 | 17:19295356 | 78112 | 17:19295499 | 78113 | 17:19295504 | 78133 | 17:19689334 | 78153 | 17:20218072 |
| 78173 | 17:20920601 | 78177 | 17:21089322 | 78195 | 17:21650017 | 78203 | 17:21824364 | 78259 | 17:24078894 |
| 78278 | 17:24557966 | 78308 | 17:25485693 | 78320 | 17:25835294 | 78333 | 17:26208169 | 78336 | 17:26275689 |
| 78338 | 17:26313864 | 78347 | 17:26586857 | 78355 | 17:26669513 | 78379 | 17:27150917 | 78417 | 17:28123761 |
| 78429 | 17:28336908 | 78457 | 17:29248479 | 78514 | 17:30796206 | 78543 | 17:31545528 | 78556 | 17:31865010 |
| 78558 | 17:31877966 | 78579 | 17:32478146 | 78583 | 17:32532692 | 78596 | 17:32813643 | 78625 | 17:33559323 |
| 78633 | 17:33630931 | 78661 | 17:34398154 | 78696 | 17:35542521 | 78722 | 17:36254116 | 78747 | 17:37178207 |
| 78773 | 17:38080956 | 78783 | 17:38278802 | 78789 | 17:38562984 | 78797 | 17:38814783 | 78824 | 17:39676479 |
| 78825 | 17:39677255 | 78826 | 17:39679094 | 78832 | 17:39827471 | 78833 | 17:39828115 | 78834 | 17:39828525 |
| 78836 | 17:39840626 | 78838 | 17:39849566 | 78839 | 17:39854144 | 78840 | 17:39858951 | 78843 | 17:39882893 |
| 78844 | 17:39883222 | 78845 | 17:39885556 | 78846 | 17:39886164 | 78847 | 17:39887756 | 78848 | 17:39893701 |
| 78849 | 17:39894975 | 78850 | 17:39896327 | 78851 | 17:39896972 | 78877 | 17:40559838 | 78880 | 17:40569673 |
| 78881 | 17:40569857 | 78882 | 17:40570434 | 78885 | 17:40597210 | 78889 | 17:40687599 | 78891 | 17:40702874 |
| 78893 | 17:40727620 | 78924 | 17:41582600 | 78993 | 17:43272612 | 78996 | 17:43327605 | 79034 | 17:44279469 |
| 79079 | 17:45388796 | 79090 | 17:45567473 | 79117 | 17:46182697 | 79173 | 17:47495174 | 79183 | 17:47822968 |
| 79184 | 17:47826553 | 79203 | 17:48325513 | 79216 | 17:48653000 | 79227 | 17:48892420 | 79233 | 17:49128208 |
| 79234 | 17:49131387 | 79272 | 17:49921719 | 79309 | 17:50714394 | 79310 | 17:50720459 | 79311 | 17:50722838 |
| 79312 | 17:50723471 | 79313 | 17:50727878 | 79314 | 17:50730200 | 79315 | 17:50731825 | 79317 | 17:50814958 |
| 79357 | 17:51667657 | 79381 | 17:52060992 | 79385 | 17:52081616 | 79386 | 17:52082865 | 79387 | 17:52087837 |
| 79388 | 17:52087901 | 79424 | 17:53278122 | 79462 | 17:54135006 | 79538 | 17:56024288 | 79550 | 17:56299499 |
| 79555 | 17:56435818 | 79572 | 17:57052924 | 79586 | 17:57342574 | 79605 | 17:57757425 | 79607 | 17:57774071 |
| 79611 | 17:57814505 | 79628 | 17:58146798 | 79636 | 17:58314278 | 79637 | 17:58317198 | 79638 | 17:58321497 |
| 79639 | 17:58322279 | 79640 | 17:58323397 | 79641 | 17:58324863 | 79642 | 17:58325379 | 79643 | 17:58325583 |
| 79644 | 17:58325642 | 79645 | 17:58325662 | 79646 | 17:58325692 | 79647 | 17:58325868 | 79648 | 17:58325926 |
| 79649 | 17:58326148 | 79650 | 17:58330227 | 79651 | 17:58331401 | 79653 | 17:58334167 | 79654 | 17:58335083 |
| 79655 | 17:58335597 | 79656 | 17:58337576 | 79657 | 17:58338335 | 79681 | 17:59019311 | 79699 | 17:59378547 |
| 79701 | 17:59392366 | 79712 | 17:59683258 | 79747 | 17:60329105 | 79749 | 17:60358707 | 79757 | 17:60444808 |
| 79798 | 17:61041921 | 79820 | 17:61336784 | 79824 | 17:61432513 | 79831 | 17:61511820 | 79893 | 17:63198489 |
| 79977 | 17:64661316 | 79989 | 17:64973779 | 79994 | 17:65038744 | 80026 | 17:65697499 | 80041 | 17:65972365 |
| 80051 | 17:66165602 | 80052 | 17:66169310 | 80053 | 17:66170354 | 80054 | 17:66171241 | 80055 | 17:66171742 |
| 80056 | 17:66172830 | 80071 | 17:66374177 | 80113 | 17:67428489 | 80128 | 17:67720645 | 80131 | 17:67842378 |
| 80139 | 17:68113867 | 80189 | 17:69059007 | 80213 | 17:69580013 | 80223 | 17:69757640 | 80227 | 17:69882555 |
| 80246 | 17:70318401 | 80276 | 17:70659081 | 80282 | 17:70929780 | 80318 | 17:71539730 | 80327 | 17:71761848 |
| 80331 | 17:71853596 | 80332 | 17:71855169 | 80333 | 17:71859405 | 80335 | 17:71900536 | 80406 | 18:712517 |
| 80420 | 18:990363 | 80449 | 18:1484040 | 80458 | 18:1637197 | 80473 | 18:1743436 | 80501 | 18:1802242 |
| 80511 | 18:2001978 | 80533 | 18:2557098 | 80550 | 18:2832635 | 80557 | 18:2935768 | 80565 | 18:3102978 |
| 80575 | 18:3297749 | 80603 | 18:4036722 | 80612 | 18:4219952 | 80613 | 18:4226405 | 80620 | 18:4439946 |
| 80628 | 18:4565913 | 80644 | 18:4871817 | 80669 | 18:5415676 | 80670 | 18:5415711 | 80671 | 18:5416566 |
| 80712 | 18:6220320 | 80723 | 18:6398640 | 80725 | 18:6407051 | 80726 | 18:6407842 | 80727 | 18:6407856 |
| 80728 | 18:6408160 | 80729 | 18:6409137 | 80730 | 18:6409528 | 80748 | 18:6759745 | 80753 | 18:6859408 |
| 80770 | 18:7273399 | 80793 | 18:7769343 | 80802 | 18:7953745 | 80851 | 18:8931926 | 80872 | 18:9156689 |
| 80884 | 18:9413940 | 80885 | 18:9414216 | 80909 | 18:9827683 | 80924 | 18:10096838 | 80935 | 18:10422385 |
| 80980 | 18:11195149 | 81051 | 18:13077929 | 81083 | 18:13931354 | 81085 | 18:13962063 | 81086 | 18:13962211 |
| 81094 | 18:14131401 | 81128 | 18:15337353 | 81175 | 18:16420426 | 81195 | 18:16937183 | 81202 | 18:17081163 |
| 81213 | 18:17191494 | 81240 | 18:17819065 | 81249 | 18:18041884 | 81257 | 18:18196329 | 81262 | 18:18274003 |
| 81263 | 18:18274911 | 81264 | 18:18279929 | 81265 | 18:18284551 | 81266 | 18:18285334 | 81268 | 18:18287100 |
| 81270 | 18:18297472 | 81271 | 18:18300144 | 81289 | 18:18654230 | 81301 | 18:18883470 | 81336 | 18:19805409 |
| 81365 | 18:20391312 | 81369 | 18:20532028 | 81370 | 18:20581067 | 81371 | 18:20586213 | 81372 | 18:20591204 |
| 81373 | 18:20594221 | 81383 | 18:20851094 | 81434 | 18:22045866 | 81499 | 18:23267678 | 81519 | 18:23730169 |
| 81533 | 18:24068173 | 81584 | 18:25002158 | 81600 | 18:25402965 | 81616 | 18:25722314 | 81657 | 18:26620395 |
| 81660 | 18:26643719 | 81667 | 18:26781973 | 81699 | 18:27670793 | 81722 | 18:28212436 | 81754 | 18:29332445 |
| 81762 | 18:29394581 | 81775 | 18:29615496 | 81779 | 18:29751573 | 81780 | 18:29770581 | 81825 | 18:31291350 |
| 81850 | 18:31971311 | 81863 | 18:32267474 | 81869 | 18:32301839 | 81874 | 18:32426539 | 81917 | 18:33371652 |
| 81918 | 18:33381630 | 81919 | 18:33381846 | 81921 | 18:33389064 | 81930 | 18:33546416 | 81931 | 18:33559158 |
| 81956 | 18:34226859 | 82000 | 18:35324050 | 82017 | 18:35722943 | 82090 | 18:37440845 | 82096 | 18:37511943 |
| 82111 | 18:37857222 | 82117 | 18:37902748 | 82162 | 18:38995251 | 82167 | 18:39072678 | 82201 | 18:39856656 |
| 82211 | 18:40078593 | 82214 | 18:40135404 | 82268 | 18:41177791 | 82300 | 18:41952487 | 82332 | 18:42484052 |
| 82374 | 18:43224250 | 82381 | 18:43359712 | 82384 | 18:43378123 | 82402 | 18:43762058 | 82434 | 18:44688799 |
| 82436 | 18:44703246 | 82492 | 18:46177257 | 82495 | 18:46269611 | 82566 | 18:46759650 | 82608 | 18:47374533 |
| 82675 | 18:48506540 | 82731 | 18:49938198 | 82779 | 18:51259647 | 82786 | 18:51451371 | 82787 | 18:51452185 |
| 82788 | 18:51455635 | 82792 | 18:51522776 | 82795 | 18:51579528 | 82817 | 18:51820303 | 82847 | 18:52482506 |
| 82860 | 18:52950856 | 82873 | 18:53350033 | 82882 | 18:53554813 | 82896 | 18:53718739 | 82916 | 18:54039068 |
| 82934 | 18:54414979 | 82973 | 18:55042223 | 83009 | 18:55891884 | 83050 | 18:56619470 | 83056 | 18:56702388 |
| 83061 | 18:56777892 | 83063 | 18:56780574 | 83064 | 18:56781239 | 83065 | 18:56788137 | 83066 | 18:56788537 |
| 83067 | 18:56790131 | 83068 | 18:56793472 | 83069 | 18:56793653 | 83070 | 18:56802310 | 83071 | 18:56802366 |
| 83072 | 18:56802408 | 83073 | 18:56803155 | 83074 | 18:56805465 | 83075 | 18:56805669 | 83076 | 18:56805763 |
| 83077 | 18:56805884 | 83078 | 18:56805908 | 83079 | 18:56805969 | 83080 | 18:56806452 | 83081 | 18:56806522 |
| 83082 | 18:56806817 | 83083 | 18:56807069 | 83084 | 18:56807143 | 83085 | 18:56807200 | 83086 | 18:56808520 |
| 83087 | 18:56808693 | 83088 | 18:56808739 | 83089 | 18:56809175 | 83090 | 18:56809229 | 83091 | 18:56809274 |
| 83092 | 18:56809314 | 83093 | 18:56809399 | 83094 | 18:56811400 | 83095 | 18:56816836 | 83096 | 18:56817529 |
| 83097 | 18:56817530 | 83098 | 18:56817556 | 83099 | 18:56817983 | 83100 | 18:56819032 | 83102 | 18:56819579 |
| 83103 | 18:56820099 | 83104 | 18:56820288 | 83105 | 18:56820485 | 83106 | 18:56821287 | 83129 | 18:57480828 |
| 83131 | 18:57489304 | 83132 | 18:57496016 | 83135 | 18:57545249 | 83136 | 18:57549761 | 83137 | 18:57559274 |
| 83154 | 18:57826436 | 83155 | 18:57826437 | 83164 | 18:57929781 | 83230 | 18:58590500 | 83236 | 18:58874702 |
| 83262 | 18:60020922 | 83277 | 18:60611535 | 83278 | 18:60661061 | 83292 | 18:61082028 | 83295 | 18:61224849 |
| 83317 | 18:61691526 | 83377 | 18:62817960 | 83416 | 18:63491911 | 83425 | 18:63743739 | 83431 | 18:63828080 |
| 83432 | 18:63835095 | 83437 | 18:63865767 | 83560 | 18:65350149 | 83571 | 18:65487247 | 83589 | 19:153194 |
| 83605 | 19:905203 | 83670 | 19:3029129 | 83693 | 19:3751571 | 83731 | 19:4866448 | 83744 | 19:5165734 |
| 83761 | 19:5576963 | 83775 | 19:5960251 | 83792 | 19:6497947 | 83798 | 19:6642039 | 83815 | 19:6924319 |
| 83883 | 19:8123606 | 83898 | 19:8310681 | 83901 | 19:8332089 | 83902 | 19:8333798 | 83903 | 19:8344332 |
| 83904 | 19:8344354 | 83905 | 19:8356008 | 83906 | 19:8366630 | 83907 | 19:8366749 | 83916 | 19:8476307 |
| 83918 | 19:8521460 | 83930 | 19:8822288 | 83958 | 19:9228505 | 83982 | 19:9886007 | 83992 | 19:10035802 |
| 83998 | 19:10157460 | 83999 | 19:10157552 | 84100 | 19:12546324 | 84101 | 19:12551345 | 84104 | 19:12587276 |
| 84139 | 19:13390721 | 84143 | 19:13523234 | 84144 | 19:13523312 | 84145 | 19:13523394 | 84146 | 19:13523599 |
| 84147 | 19:13523669 | 84148 | 19:13523920 | 84149 | 19:13524217 | 84150 | 19:13524534 | 84151 | 19:13524690 |
| 84152 | 19:13525738 | 84191 | 19:14079144 | 84213 | 19:14542746 | 84214 | 19:14547882 | 84228 | 19:14975147 |
| 84229 | 19:14976930 | 84230 | 19:14977190 | 84240 | 19:15153935 | 84243 | 19:15176665 | 84277 | 19:15979571 |
| 84333 | 19:16959557 | 84403 | 19:18344112 | 84406 | 19:18364792 | 84491 | 19:20068707 | 84575 | 19:21354124 |
| 84635 | 19:22407739 | 84683 | 19:23510771 | 84701 | 19:23901688 | 84718 | 19:24121007 | 84738 | 19:24347182 |
| 84800 | 19:25069906 | 84803 | 19:25117527 | 84804 | 19:25118864 | 84812 | 19:25238500 | 84840 | 19:25573317 |
| 84841 | 19:25583861 | 84842 | 19:25635561 | 84880 | 19:26078995 | 84883 | 19:26146723 | 84884 | 19:26158447 |
| 84885 | 19:26162940 | 84886 | 19:26166792 | 84887 | 19:26177724 | 84888 | 19:26180605 | 84899 | 19:26370592 |
| 84901 | 19:26384973 | 84902 | 19:26386173 | 84904 | 19:26386998 | 84905 | 19:26408649 | 84906 | 19:26414194 |
| 84907 | 19:26425088 | 84908 | 19:26428880 | 84910 | 19:26432937 | 84912 | 19:26434534 | 84916 | 19:26440887 |
| 84917 | 19:26442377 | 84918 | 19:26442417 | 84922 | 19:26458156 | 84925 | 19:26462109 | 84928 | 19:26466738 |
| 84930 | 19:26468564 | 84931 | 19:26485885 | 84932 | 19:26489724 | 84936 | 19:26508139 | 84939 | 19:26510614 |
| 84942 | 19:26555053 | 84944 | 19:26575215 | 84945 | 19:26588232 | 84946 | 19:26595700 | 84947 | 19:26595755 |
| 84948 | 19:26599010 | 84950 | 19:26628925 | 84951 | 19:26662321 | 84953 | 19:26675470 | 84954 | 19:26676204 |
| 84956 | 19:26697210 | 84958 | 19:26705636 | 84959 | 19:26707790 | 84960 | 19:26719756 | 84961 | 19:26732472 |
| 84963 | 19:26744245 | 85040 | 19:28793496 | 85083 | 19:29602130 | 85084 | 19:29603381 | 85102 | 19:29967023 |
| 85123 | 19:30488002 | 85130 | 19:30639326 | 85131 | 19:30644348 | 85132 | 19:30645410 | 85133 | 19:30648555 |
| 85134 | 19:30696412 | 85137 | 19:30818025 | 85139 | 19:30826714 | 85167 | 19:31463142 | 85168 | 19:31485672 |
| 85183 | 19:31907703 | 85187 | 19:31968664 | 85207 | 19:32399697 | 85219 | 19:32665116 | 85236 | 19:33046409 |
| 85285 | 19:34025535 | 85395 | 19:35866723 | 85400 | 19:35970366 | 85434 | 19:36329939 | 85487 | 19:37333280 |
| 85581 | 19:40048911 | 85595 | 19:40251758 | 85597 | 19:40279227 | 85615 | 19:40640928 | 85635 | 19:40995967 |
| 85654 | 19:41486757 | 85668 | 19:41704492 | 85669 | 19:41719174 | 85686 | 19:41943823 | 85702 | 19:42161619 |
| 85726 | 19:42580737 | 85734 | 19:42764834 | 85760 | 19:43424335 | 85761 | 19:43424535 | 85762 | 19:43428799 |
| 85763 | 19:43431509 | 85764 | 19:43433116 | 85765 | 19:43435751 | 85766 | 19:43438458 | 85770 | 19:43493558 |
| 85778 | 19:43722064 | 85864 | 19:45668089 | 85868 | 19:45717594 | 85869 | 19:45717924 | 85870 | 19:45717976 |
| 85871 | 19:45717979 | 85872 | 19:45718107 | 85873 | 19:45718307 | 85874 | 19:45718340 | 85875 | 19:45720202 |
| 85876 | 19:45720264 | 85877 | 19:45720488 | 85878 | 19:45720862 | 85879 | 19:45736048 | 85880 | 19:45738664 |
| 85881 | 19:45740485 | 85882 | 19:45741144 | 85883 | 19:45741389 | 85884 | 19:45741952 | 85885 | 19:45742152 |
| 85886 | 19:45742153 | 85894 | 19:45834449 | 85995 | 19:48080018 | 86018 | 19:48469710 | 86020 | 19:48505288 |
| 86052 | 19:49235212 | 86059 | 19:49331336 | 86060 | 19:49332593 | 86117 | 19:50655487 | 86124 | 19:50694652 |
| 86125 | 19:50699155 | 86126 | 19:50703436 | 86127 | 19:50711021 | 86128 | 19:50712302 | 86129 | 19:50714204 |
| 86130 | 19:50715174 | 86131 | 19:50716477 | 86132 | 19:50718075 | 86133 | 19:50724757 | 86134 | 19:50732339 |
| 86135 | 19:50734637 | 86136 | 19:50741020 | 86137 | 19:50772016 | 86210 | 19:51559279 | 86211 | 19:51559359 |
| 86239 | 19:52037026 | 86255 | 19:52551695 | 86317 | 19:53746270 | 86327 | 19:53883618 | 86328 | 19:53883934 |
| 86336 | 19:54027650 | 86340 | 19:54047174 | 86359 | 19:54403366 | 86370 | 19:54621153 | 86373 | 19:54664915 |
| 86374 | 19:54665643 | 86375 | 19:54667304 | 86377 | 19:54672612 | 86392 | 19:54949422 | 86397 | 19:55036335 |
| 86412 | 19:55420287 | 86545 | 19:56454067 | 86547 | 19:56460077 | 86568 | 19:56726108 | 86584 | 19:57037210 |
| 86601 | 19:57419304 | 86605 | 19:57490381 | 86606 | 19:57508171 | 86615 | 19:57670291 | 86624 | 19:57908956 |
| 86637 | 19:58189519 | 86654 | 19:58502690 | 86661 | 19:58677753 | 86662 | 19:58689620 | 86691 | 19:59301702 |
| 86694 | 19:59353477 | 86714 | 19:59622681 | 86730 | 19:59843660 | 86735 | 19:59905716 | 86755 | 19:60165330 |
| 86757 | 19:60175972 | 86758 | 19:60176459 | 86760 | 19:60192077 | 86784 | 19:60532635 | 86793 | 19:60643681 |
| 86797 | 19:60699603 | 86803 | 19:60819634 | 86816 | 19:61121915 | 86853 | 19:61536597 | 86863 | 19:61642528 |
| 86864 | 19:61642559 | 86875 | 19:61831896 | 86896 | 19:62236608 | 86901 | 19:62290414 | 86906 | 19:62421264 |
| 86921 | 19:62751964 | 86923 | 19:62757779 | 86937 | 19:62914703 | 86971 | 20:465779 | 86984 | 20:581428 |
| 86985 | 20:583434 | 87026 | 20:1587207 | 87034 | 20:1637477 | 87046 | 20:1840861 | 87049 | 20:1872065 |
| 87081 | 20:2302262 | 87098 | 20:2623153 | 87109 | 20:3049545 | 87110 | 20:3050301 | 87111 | 20:3052518 |
| 87112 | 20:3056100 | 87113 | 20:3056394 | 87114 | 20:3061100 | 87115 | 20:3061457 | 87116 | 20:3062741 |
| 87117 | 20:3064116 | 87118 | 20:3064279 | 87119 | 20:3065002 | 87120 | 20:3065571 | 87121 | 20:3065997 |
| 87185 | 20:4419534 | 87443 | 20:5918697 | 87517 | 20:7068652 | 87549 | 20:7706966 | 87560 | 20:7984208 |
| 87598 | 20:8931603 | 87671 | 20:10580364 | 87672 | 20:10581845 | 87697 | 20:11193697 | 87724 | 20:11666353 |
| 87731 | 20:11849546 | 87737 | 20:12016234 | 87780 | 20:12743838 | 87791 | 20:13014120 | 87797 | 20:13145133 |
| 87811 | 20:13530219 | 87817 | 20:13767878 | 87836 | 20:14332360 | 87852 | 20:15273040 | 87864 | 20:15564869 |
| 87875 | 20:15869027 | 87906 | 20:16621914 | 87909 | 20:16650192 | 87961 | 20:18107968 | 87991 | 20:19026521 |
| 87993 | 20:19083370 | 88011 | 20:19606439 | 88019 | 20:19755587 | 88078 | 20:21474908 | 88083 | 20:21584291 |
| 88117 | 20:22063416 | 88191 | 20:22598687 | 88200 | 20:22757553 | 88209 | 20:22931278 | 88259 | 20:23864507 |
| 88277 | 20:23991980 | 88323 | 20:25147490 | 88359 | 20:26491689 | 88360 | 20:26496401 | 88361 | 20:26502059 |
| 88362 | 20:26504123 | 88363 | 20:26514971 | 88364 | 20:26518907 | 88366 | 20:26522200 | 88406 | 20:27651444 |
| 88407 | 20:27665296 | 88420 | 20:27980203 | 88434 | 20:28277863 | 88467 | 20:28789560 | 88482 | 20:29236907 |
| 88526 | 20:30411387 | 88535 | 20:30681003 | 88568 | 20:31716095 | 88572 | 20:31786758 | 88574 | 20:31871902 |
| 88685 | 20:34576290 | 88806 | 20:37374849 | 88817 | 20:37489422 | 88837 | 20:37861824 | 88857 | 20:38404986 |
| 88885 | 20:39228116 | 88889 | 20:39268509 | 88899 | 20:39457698 | 88902 | 20:39467997 | 88907 | 20:39535650 |
| 88913 | 20:39565695 | 88931 | 20:39982244 | 88942 | 20:40219777 | 88959 | 20:40746359 | 88976 | 20:41050966 |
| 89002 | 20:41628336 | 89045 | 20:42462021 | 89117 | 20:44485586 | 89140 | 20:45144462 | 89147 | 20:45457044 |
| 89148 | 20:45547831 | 89150 | 20:45567543 | 89154 | 20:45642750 | 89156 | 20:45675568 | 89158 | 20:45694391 |
| 89174 | 20:46029522 | 89175 | 20:46040337 | 89176 | 20:46040344 | 89177 | 20:46043226 | 89189 | 20:46349920 |
| 89197 | 20:46707839 | 89206 | 20:47136685 | 89214 | 20:47190891 | 89228 | 20:47736047 | 89277 | 20:49126008 |
| 89278 | 20:49182818 | 89301 | 20:49941637 | 89316 | 20:50311371 | 89348 | 20:51482831 | 89384 | 20:52684364 |
| 89407 | 20:53375334 | 89412 | 20:53445710 | 89417 | 20:53542795 | 89468 | 20:55149259 | 89470 | 20:55168267 |
| 89471 | 20:55169154 | 89474 | 20:55188763 | 89476 | 20:55206284 | 89477 | 20:55207791 | 89481 | 20:55321910 |
| 89488 | 20:55507243 | 89626 | 20:58393406 | 89647 | 20:58770472 | 89651 | 20:58809259 | 89694 | 20:59879577 |
| 89697 | 20:59895328 | 89741 | 20:61269332 | 89769 | 20:61983565 | 89777 | 20:62202768 | 89791 | 20:62523219 |
| 89834 | 20:63451088 | 89878 | 20:64194501 | 89901 | 20:64754306 | 89915 | 20:65165202 | 89925 | 20:65480230 |
| 89931 | 20:65608403 | 89992 | 20:66984199 | 90045 | 20:68049361 | 90056 | 20:68247302 | 90060 | 20:68312623 |
| 90065 | 20:68359426 | 90126 | 20:69498583 | 90156 | 20:70136618 | 90175 | 20:70711203 | 90290 | 21:560149 |
| 90307 | 21:1776376 | 90351 | 21:3330534 | 90366 | 21:3866539 | 90394 | 21:4290924 | 90412 | 21:4894437 |
| 90448 | 21:5848777 | 90485 | 21:7043405 | 90502 | 21:7479548 | 90518 | 21:7765157 | 90533 | 21:8066355 |
| 90536 | 21:8077420 | 90537 | 21:8084411 | 90539 | 21:8091687 | 90540 | 21:8093756 | 90541 | 21:8096108 |
| 90544 | 21:8098084 | 90545 | 21:8100095 | 90547 | 21:8121506 | 90548 | 21:8123911 | 90550 | 21:8132548 |
| 90564 | 21:8374844 | 90580 | 21:8819472 | 90593 | 21:9141650 | 90611 | 21:9612990 | 90614 | 21:9635812 |
| 90615 | 21:9642385 | 90658 | 21:10706242 | 90672 | 21:10964762 | 90688 | 21:11397271 | 90715 | 21:12027109 |
| 90738 | 21:12572473 | 90764 | 21:13059367 | 90801 | 21:13974032 | 90810 | 21:14159859 | 90836 | 21:14800811 |
| 90842 | 21:14976378 | 90881 | 21:15763479 | 90898 | 21:16325852 | 90899 | 21:16327666 | 90900 | 21:16352286 |
| 90902 | 21:16380714 | 90904 | 21:16401319 | 90907 | 21:16447288 | 90937 | 21:16900535 | 90938 | 21:16964578 |
| 90960 | 21:17491219 | 90980 | 21:18041395 | 91095 | 21:19926364 | 91108 | 21:20273386 | 91145 | 21:20915737 |
| 91166 | 21:21373080 | 91192 | 21:21922090 | 91199 | 21:22093113 | 91212 | 21:22196208 | 91297 | 21:24454479 |
| 91302 | 21:24572190 | 91312 | 21:24911518 | 91403 | 21:27617328 | 91405 | 21:27709157 | 91450 | 21:29090583 |
| 91467 | 21:29574040 | 91468 | 21:29585741 | 91469 | 21:29663335 | 91474 | 21:29793976 | 91529 | 21:31234804 |
| 91619 | 21:33320463 | 91621 | 21:33351272 | 91625 | 21:33376637 | 91660 | 21:33757910 | 91681 | 21:34100944 |
| 91710 | 21:34503640 | 91721 | 21:34672141 | 91744 | 21:35184835 | 91759 | 21:35593323 | 91773 | 21:35773499 |
| 91788 | 21:36015961 | 91795 | 21:36164785 | 91824 | 21:36934454 | 91828 | 21:37113932 | 91829 | 21:37118207 |
| 91830 | 21:37118300 | 91917 | 21:39931787 | 91933 | 21:40317765 | 91942 | 21:40547674 | 91996 | 21:41966823 |
| 92040 | 21:42868063 | 92065 | 21:43541273 | 92066 | 21:43549420 | 92077 | 21:43767846 | 92081 | 21:43869821 |
| 92105 | 21:44394987 | 92129 | 21:44779372 | 92150 | 21:45470174 | 92187 | 21:46513102 | 92213 | 21:47280539 |
| 92214 | 21:47282065 | 92215 | 21:47287202 | 92216 | 21:47288654 | 92217 | 21:47288689 | 92218 | 21:47289181 |
| 92220 | 21:47294064 | 92222 | 21:47296827 | 92223 | 21:47297692 | 92224 | 21:47297903 | 92225 | 21:47298236 |
| 92226 | 21:47298622 | 92227 | 21:47298661 | 92228 | 21:47298894 | 92229 | 21:47298942 | 92230 | 21:47299468 |
| 92231 | 21:47299536 | 92232 | 21:47299742 | 92233 | 21:47299950 | 92234 | 21:47300166 | 92236 | 21:47301069 |
| 92237 | 21:47301092 | 92238 | 21:47301705 | 92239 | 21:47301914 | 92240 | 21:47301979 | 92241 | 21:47302160 |
| 92242 | 21:47302405 | 92243 | 21:47302771 | 92244 | 21:47302793 | 92245 | 21:47303150 | 92246 | 21:47303326 |
| 92247 | 21:47303615 | 92248 | 21:47304710 | 92249 | 21:47308349 | 92250 | 21:47309250 | 92251 | 21:47314809 |
| 92253 | 21:47316827 | 92263 | 21:47572280 | 92286 | 21:48277772 | 92318 | 21:49185947 | 92319 | 21:49189566 |
| 92320 | 21:49193705 | 92321 | 21:49194179 | 92323 | 21:49195976 | 92324 | 21:49209645 | 92326 | 21:49218212 |
| 92327 | 21:49229215 | 92328 | 21:49231400 | 92360 | 21:50042557 | 92372 | 21:50467495 | 92407 | 21:51606765 |
| 92424 | 21:52027284 | 92433 | 21:52289877 | 92446 | 21:52553332 | 92471 | 21:53340020 | 92497 | 21:54060629 |
| 92505 | 21:54338413 | 92522 | 21:54761061 | 92523 | 21:54761327 | 92524 | 21:54766467 | 92526 | 21:54773661 |
| 92527 | 21:54779429 | 92528 | 21:54795770 | 92570 | 21:55568962 | 92571 | 21:55571357 | 92575 | 21:55603036 |
| 92577 | 21:55606404 | 92645 | 21:56889237 | 92646 | 21:56907066 | 92724 | 21:57243795 | 92757 | 21:58266418 |
| 92773 | 21:58557250 | 92819 | 21:59483269 | 92840 | 21:59828548 | 92841 | 21:59838076 | 92844 | 21:59872593 |
| 92852 | 21:59962135 | 92853 | 21:59965050 | 92861 | 21:60071151 | 92876 | 21:60290443 | 92897 | 21:60717146 |
| 92945 | 21:61871337 | 92978 | 21:62509823 | 92981 | 21:62565907 | 92997 | 21:62953263 | 93021 | 21:63719399 |
| 93022 | 21:63775744 | 93035 | 21:64020970 | 93043 | 21:64322263 | 93045 | 21:64323466 | 93054 | 21:64458851 |
| 93055 | 21:64468813 | 93061 | 21:64551281 | 93067 | 21:64674944 | 93071 | 21:64702159 | 93075 | 21:64763778 |
| 93111 | 21:65219263 | 93113 | 21:65222945 | 93120 | 21:65350726 | 93122 | 21:65389454 | 93124 | 21:65436920 |
| 93127 | 21:65495121 | 93138 | 21:65745850 | 93203 | 21:67086290 | 93205 | 21:67097791 | 93207 | 21:67137594 |
| 93216 | 21:67459394 | 93217 | 21:67461089 | 93236 | 21:67933900 | 93237 | 21:67933901 | 93267 | 21:68505684 |
| 93284 | 21:68792488 | 93291 | 21:68917772 | 93302 | 21:69177495 | 93307 | 21:69279748 | 93384 | 22:1336082 |
| 93421 | 22:2092658 | 93448 | 22:2744263 | 93451 | 22:2759833 | 93452 | 22:2768731 | 93476 | 22:3271155 |
| 93490 | 22:3549590 | 93492 | 22:3565222 | 93512 | 22:4182249 | 93517 | 22:4255887 | 93535 | 22:4575207 |
| 93596 | 22:5655551 | 93689 | 22:7518330 | 93690 | 22:7526334 | 93700 | 22:7714187 | 93721 | 22:8392537 |
| 93725 | 22:8430070 | 93741 | 22:9021813 | 93749 | 22:9259333 | 93782 | 22:9998563 | 93798 | 22:10114240 |
| 93824 | 22:10733620 | 93845 | 22:11198997 | 93857 | 22:11572590 | 93875 | 22:11958308 | 93895 | 22:12274925 |
| 93927 | 22:12850291 | 93957 | 22:13771925 | 93999 | 22:14949092 | 94000 | 22:14961326 | 94003 | 22:15026827 |
| 94021 | 22:15501897 | 94034 | 22:15667257 | 94037 | 22:15711517 | 94056 | 22:16388677 | 94061 | 22:16514054 |
| 94079 | 22:16739297 | 94121 | 22:17893434 | 94128 | 22:18003077 | 94129 | 22:18033855 | 94139 | 22:18259710 |
| 94140 | 22:18265709 | 94154 | 22:18559489 | 94166 | 22:18849789 | 94167 | 22:18851178 | 94178 | 22:19237876 |
| 94184 | 22:19372836 | 94197 | 22:19837982 | 94198 | 22:19847979 | 94213 | 22:20176939 | 94229 | 22:20643619 |
| 94230 | 22:20646235 | 94241 | 22:20897046 | 94278 | 22:21648235 | 94280 | 22:21664858 | 94296 | 22:21953904 |
| 94303 | 22:22190483 | 94309 | 22:22421295 | 94314 | 22:22604015 | 94324 | 22:22885054 | 94352 | 22:23611781 |
| 94362 | 22:23913434 | 94413 | 22:25649715 | 94420 | 22:25917253 | 94440 | 22:26167092 | 94456 | 22:26660485 |
| 94489 | 22:27621315 | 94505 | 22:28117808 | 94510 | 22:28166101 | 94521 | 22:28401484 | 94530 | 22:28491057 |
| 94568 | 22:29357579 | 94579 | 22:29669451 | 94592 | 22:29900170 | 94598 | 22:30126912 | 94643 | 22:31208133 |
| 94672 | 22:32149285 | 94703 | 22:33059262 | 94715 | 22:33350762 | 94739 | 22:34116071 | 94741 | 22:34136171 |
| 94756 | 22:34692656 | 94757 | 22:34695203 | 94773 | 22:35027110 | 94777 | 22:35094672 | 94779 | 22:35111999 |
| 94789 | 22:35301748 | 94814 | 22:35855583 | 94827 | 22:36060507 | 94829 | 22:36134170 | 94835 | 22:36318773 |
| 94861 | 22:37054760 | 94868 | 22:37235828 | 94896 | 22:37669398 | 94913 | 22:38264582 | 94914 | 22:38283974 |
| 94923 | 22:38466243 | 94937 | 22:38695645 | 94941 | 22:38735989 | 94942 | 22:38740955 | 94960 | 22:39188813 |
| 94989 | 22:39567152 | 94995 | 22:39656245 | 94997 | 22:39682658 | 95055 | 22:41016011 | 95066 | 22:41417903 |
| 95081 | 22:41777356 | 95084 | 22:41792218 | 95085 | 22:41797029 | 95089 | 22:41877932 | 95149 | 22:43286201 |
| 95155 | 22:43386150 | 95178 | 22:44035160 | 95210 | 22:44640940 | 95237 | 22:45552005 | 95247 | 22:45691255 |
| 95263 | 22:45913562 | 95274 | 22:46107748 | 95276 | 22:46144763 | 95297 | 22:46746909 | 95317 | 22:47040045 |
| 95323 | 22:47111303 | 95426 | 22:49820256 | 95433 | 22:49940593 | 95434 | 22:49970190 | 95440 | 22:50067364 |
| 95499 | 22:51502260 | 95500 | 22:51503045 | 95531 | 22:52333167 | 95540 | 22:52556549 | 95558 | 22:52884674 |
| 95565 | 22:52961463 | 95592 | 22:53497101 | 95621 | 22:54011562 | 95672 | 22:55011602 | 95677 | 22:55091224 |
| 95695 | 22:55323748 | 95712 | 22:55563765 | 95758 | 22:56293581 | 95793 | 22:56540115 | 95794 | 22:56540668 |
| 95795 | 22:56542873 | 95796 | 22:56544054 | 95798 | 22:56573939 | 95850 | 22:57521486 | 95882 | 22:57942911 |
| 95894 | 22:58136676 | 95910 | 22:58494061 | 95920 | 22:58678398 | 95922 | 22:58700947 | 95924 | 22:58773917 |
| 95941 | 22:59128229 | 95967 | 22:59629485 | 95973 | 22:59712330 | 95985 | 22:59846886 | 96044 | 23:774985 |
| 96050 | 23:968798 | 96057 | 23:1194189 | 96063 | 23:1325388 | 96069 | 23:1434392 | 96095 | 23:2175010 |
| 96116 | 23:2637687 | 96164 | 23:3563217 | 96210 | 23:4375629 | 96230 | 23:4759481 | 96241 | 23:4872845 |
| 96246 | 23:5028069 | 96328 | 23:6757709 | 96342 | 23:7060820 | 96378 | 23:7879887 | 96380 | 23:7903756 |
| 96398 | 23:8328018 | 96449 | 23:9180909 | 96470 | 23:9852522 | 96499 | 23:10263390 | 96518 | 23:10703356 |
| 96522 | 23:10786602 | 96547 | 23:11354447 | 96568 | 23:11793160 | 96578 | 23:12035181 | 96579 | 23:12038531 |
| 96580 | 23:12039512 | 96582 | 23:12047337 | 96621 | 23:12878298 | 96665 | 23:13769527 | 96690 | 23:14024594 |
| 96691 | 23:14041498 | 96693 | 23:14067842 | 96705 | 23:14437275 | 96707 | 23:14454128 | 96722 | 23:14695783 |
| 96724 | 23:14770597 | 96737 | 23:14938161 | 96742 | 23:15032946 | 96784 | 23:15623072 | 96791 | 23:15668311 |
| 96800 | 23:15846192 | 96807 | 23:16055984 | 96852 | 23:17393860 | 96860 | 23:17578001 | 96864 | 23:17628981 |
| 96866 | 23:17657910 | 96905 | 23:18560341 | 96911 | 23:18766462 | 96916 | 23:18918643 | 96964 | 23:19690687 |
| 96967 | 23:19754433 | 96969 | 23:19762716 | 96978 | 23:19935139 | 96980 | 23:19938929 | 96995 | 23:20172115 |
| 97011 | 23:20449980 | 97015 | 23:20553981 | 97016 | 23:20554380 | 97017 | 23:20555345 | 97018 | 23:20555464 |
| 97019 | 23:20555512 | 97020 | 23:20555877 | 97046 | 23:21266809 | 97053 | 23:21371538 | 97074 | 23:22025892 |
| 97082 | 23:22189816 | 97084 | 23:22253261 | 97099 | 23:22609075 | 97100 | 23:22632358 | 97120 | 23:23494563 |
| 97121 | 23:23495169 | 97122 | 23:23515623 | 97128 | 23:23599173 | 97131 | 23:23663060 | 97133 | 23:23729292 |
| 97144 | 23:23841412 | 97166 | 23:24310745 | 97182 | 23:24584592 | 97220 | 23:24825942 | 97235 | 23:24996122 |
| 97236 | 23:25010458 | 97276 | 23:25343657 | 97294 | 23:25495218 | 97307 | 23:25727838 | 97371 | 23:27168873 |
| 97396 | 23:27404973 | 97400 | 23:27427125 | 97416 | 23:27534819 | 97418 | 23:27545798 | 97509 | 23:28274177 |
| 97515 | 23:28328479 | 97516 | 23:28329217 | 97524 | 23:28449487 | 97546 | 23:28783122 | 97592 | 23:29113119 |
| 97631 | 23:30057510 | 97657 | 23:30485309 | 97658 | 23:30487092 | 97701 | 23:31528534 | 97733 | 23:32121998 |
| 97741 | 23:32302519 | 97742 | 23:32303395 | 97743 | 23:32303732 | 97744 | 23:32304775 | 97745 | 23:32304794 |
| 97746 | 23:32305503 | 97748 | 23:32348823 | 97818 | 23:33768810 | 97821 | 23:33852032 | 97853 | 23:34945913 |
| 97854 | 23:34946818 | 97855 | 23:34946868 | 97856 | 23:34948119 | 97858 | 23:35009819 | 97867 | 23:35427374 |
| 97893 | 23:36297029 | 97899 | 23:36392895 | 97941 | 23:37537593 | 97944 | 23:37550813 | 97949 | 23:37653049 |
| 97985 | 23:38817188 | 97986 | 23:38818425 | 98075 | 23:39639975 | 98079 | 23:39681488 | 98094 | 23:39977867 |
| 98134 | 23:40742268 | 98190 | 23:41726170 | 98215 | 23:42235597 | 98220 | 23:42301353 | 98255 | 23:43110372 |
| 98280 | 23:43675243 | 98300 | 23:44151531 | 98315 | 23:44315474 | 98337 | 23:44789129 | 98433 | 23:46714094 |
| 98477 | 23:47504887 | 98478 | 23:47505469 | 98507 | 23:47898321 | 98510 | 23:47934746 | 98513 | 23:47974902 |
| 98545 | 23:48734960 | 98548 | 23:48756252 | 98551 | 23:48811144 | 98563 | 23:49008700 | 98572 | 23:49166634 |
| 98603 | 23:49594224 | 98626 | 23:49959432 | 98629 | 23:50008283 | 98643 | 23:50262003 | 98652 | 23:50392532 |
| 98685 | 23:51016737 | 98702 | 23:51334319 | 98749 | 23:52192597 | 98751 | 23:52225791 | 98752 | 23:52234534 |
| 98753 | 23:52235518 | 98768 | 24:90983 | 98820 | 24:1186935 | 98822 | 24:1209847 | 98833 | 24:1549261 |
| 98855 | 24:1914090 | 98878 | 24:2417031 | 98920 | 24:3326601 | 98976 | 24:4463662 | 98988 | 24:4937656 |
| 98990 | 24:4964946 | 98992 | 24:4997353 | 99007 | 24:5345133 | 99010 | 24:5453007 | 99011 | 24:5453574 |
| 99019 | 24:5665620 | 99110 | 24:8068578 | 99117 | 24:8174902 | 99126 | 24:8403392 | 99156 | 24:9223598 |
| 99164 | 24:9437183 | 99183 | 24:9782804 | 99185 | 24:9813237 | 99187 | 24:9859970 | 99197 | 24:10080656 |
| 99198 | 24:10082192 | 99199 | 24:10083376 | 99200 | 24:10084233 | 99201 | 24:10085368 | 99202 | 24:10085413 |
| 99203 | 24:10085426 | 99204 | 24:10085505 | 99205 | 24:10085865 | 99206 | 24:10086080 | 99207 | 24:10086129 |
| 99208 | 24:10086191 | 99209 | 24:10087002 | 99210 | 24:10087384 | 99211 | 24:10087677 | 99212 | 24:10088340 |
| 99213 | 24:10089300 | 99214 | 24:10090958 | 99215 | 24:10092604 | 99216 | 24:10092904 | 99217 | 24:10093339 |
| 99218 | 24:10093371 | 99219 | 24:10093377 | 99220 | 24:10093477 | 99221 | 24:10093552 | 99222 | 24:10093626 |
| 99223 | 24:10099234 | 99224 | 24:10100942 | 99225 | 24:10101971 | 99226 | 24:10102022 | 99227 | 24:10102315 |
| 99228 | 24:10102992 | 99229 | 24:10104790 | 99230 | 24:10106712 | 99231 | 24:10112256 | 99232 | 24:10120280 |
| 99233 | 24:10120318 | 99234 | 24:10125493 | 99235 | 24:10127888 | 99236 | 24:10128409 | 99237 | 24:10128428 |
| 99238 | 24:10128467 | 99239 | 24:10129710 | 99240 | 24:10129815 | 99241 | 24:10131022 | 99242 | 24:10131061 |
| 99243 | 24:10131076 | 99244 | 24:10131213 | 99245 | 24:10131371 | 99246 | 24:10131470 | 99247 | 24:10131562 |
| 99248 | 24:10132898 | 99249 | 24:10132907 | 99250 | 24:10134379 | 99251 | 24:10134487 | 99252 | 24:10134521 |
| 99253 | 24:10134705 | 99254 | 24:10134719 | 99255 | 24:10134821 | 99256 | 24:10134859 | 99257 | 24:10134952 |
| 99258 | 24:10134953 | 99259 | 24:10136095 | 99260 | 24:10137902 | 99261 | 24:10137983 | 99262 | 24:10139078 |
| 99263 | 24:10139760 | 99264 | 24:10140006 | 99265 | 24:10140938 | 99266 | 24:10143195 | 99267 | 24:10143925 |
| 99268 | 24:10144011 | 99269 | 24:10144116 | 99270 | 24:10150408 | 99271 | 24:10151986 | 99272 | 24:10153446 |
| 99273 | 24:10155422 | 99291 | 24:10548737 | 99303 | 24:10838109 | 99313 | 24:11081707 | 99347 | 24:11895230 |
| 99374 | 24:12693269 | 99378 | 24:12740073 | 99397 | 24:13255685 | 99401 | 24:13395143 | 99406 | 24:13474854 |
| 99411 | 24:13599253 | 99431 | 24:14202286 | 99432 | 24:14280523 | 99460 | 24:14990004 | 99467 | 24:15215610 |
| 99471 | 24:15361519 | 99472 | 24:15366916 | 99495 | 24:16259312 | 99498 | 24:16327453 | 99514 | 24:16832720 |
| 99517 | 24:16888113 | 99550 | 24:17884468 | 99553 | 24:17978572 | 99564 | 24:18322902 | 99588 | 24:18993346 |
| 99621 | 24:20085016 | 99710 | 24:21387050 | 99713 | 24:21396719 | 99714 | 24:21407863 | 99721 | 24:21479759 |
| 99823 | 24:24049399 | 99835 | 24:24317905 | 99862 | 24:24997696 | 99899 | 24:25782726 | 99918 | 24:26095475 |
| 99926 | 24:26225994 | 99966 | 24:27495986 | 10000 | 24:28406831 | 10000 | 24:28544002 | 10000 | 24:28549242 |
| 10002 | 24:28829549 | 10002 | 24:29046077 | 10007 | 24:30123139 | 10019 | 24:32865289 | 10024 | 24:34044145 |
| 10025 | 24:34324032 | 10028 | 24:35161423 | 10029 | 24:35363476 | 10031 | 24:35787989 | 10034 | 24:36327209 |
| 10034 | 24:36389875 | 10043 | 24:39349798 | 10047 | 24:40319050 | 10048 | 24:40319355 | 10048 | 24:40320188 |
| 10048 | 24:40320243 | 10048 | 24:40320244 | 10048 | 24:40320423 | 10048 | 24:40320519 | 10048 | 24:40320531 |
| 10048 | 24:40320604 | 10048 | 24:40320642 | 10048 | 24:40320662 | 10049 | 24:40346626 | 10049 | 24:40351905 |
| 10062 | 24:43050450 | 10062 | 24:43264177 | 10064 | 24:43757869 | 10064 | 24:44070040 | 10065 | 24:44312012 |
| 10066 | 24:44454841 | 10067 | 24:44630854 | 10068 | 24:44900221 | 10069 | 24:45024408 | 10073 | 24:45883707 |
| 10074 | 24:45892470 | 10074 | 24:45893302 | 10074 | 24:45900861 | 10074 | 24:45906465 | 10074 | 24:45915865 |
| 10074 | 24:45916571 | 10074 | 24:45917829 | 10075 | 24:45935654 | 10075 | 24:45946887 | 10075 | 24:45961034 |
| 10075 | 24:45965027 | 10075 | 24:45966438 | 10075 | 24:45966683 | 10075 | 24:46019888 | 10076 | 24:46021234 |
| 10076 | 24:46031614 | 10076 | 24:46039025 | 10076 | 24:46039978 | 10076 | 24:46040921 | 10076 | 24:46047288 |
| 10076 | 24:46050396 | 10076 | 24:46052145 | 10079 | 24:46519527 | 10090 | 24:49563537 | 10100 | 24:51861629 |
| 10100 | 24:51877952 | 10102 | 24:52249991 | 10102 | 24:52250875 | 10103 | 24:52380242 | 10103 | 24:52572272 |
| 10103 | 24:52579310 | 10108 | 24:53806202 | 10115 | 24:55068839 | 10121 | 24:56483606 | 10121 | 24:56487277 |
| 10123 | 24:56927299 | 10123 | 24:56931903 | 10124 | 24:56935898 | 10129 | 24:58059868 | 10130 | 24:58143848 |
| 10131 | 24:58393476 | 10132 | 24:58514430 | 10133 | 24:58871566 | 10138 | 24:59956065 | 10138 | 24:59956162 |
| 10141 | 24:60385381 | 10141 | 24:60520182 | 10145 | 24:61136358 | 10147 | 24:61660672 | 1015 | 25:326413 |
| 10155 | 25:1326500 | 10157 | 25:1480308 | 10158 | 25:1751313 | 10162 | 25:2262957 | 10162 | 25:2264339 |
| 10162 | 25:2264421 | 10163 | 25:2264518 | 10163 | 25:2264598 | 10163 | 25:2264608 | 10163 | 25:2264757 |
| 10163 | 25:2264786 | 10163 | 25:2265149 | 10163 | 25:2266070 | 10163 | 25:2266258 | 10163 | 25:2266451 |
| 10163 | 25:2266559 | 10164 | 25:2266588 | 10164 | 25:2266682 | 10164 | 25:2266930 | 10164 | 25:2267017 |
| 10164 | 25:2267496 | 10164 | 25:2267627 | 10164 | 25:2267877 | 10164 | 25:2267895 | 10164 | 25:2267998 |
| 10164 | 25:2267999 | 10165 | 25:2268074 | 10165 | 25:2268874 | 10165 | 25:2269160 | 10165 | 25:2269161 |
| 10165 | 25:2269296 | 10165 | 25:2269538 | 10165 | 25:2269574 | 10165 | 25:2324283 | 10168 | 25:2565490 |
| 10175 | 25:3236023 | 10175 | 25:3277932 | 10177 | 25:3531364 | 10177 | 25:3532276 | 10179 | 25:3846717 |
| 10181 | 25:4251363 | 10181 | 25:4273072 | 10182 | 25:4417956 | 10186 | 25:5178359 | 10186 | 25:5356785 |
| 10187 | 25:5358991 | 10187 | 25:5370164 | 10187 | 25:5487714 | 10188 | 25:5507570 | 10188 | 25:5518043 |
| 10188 | 25:5518349 | 10188 | 25:5518412 | 10188 | 25:5519000 | 10188 | 25:5519033 | 10188 | 25:5519088 |
| 10188 | 25:5519241 | 10188 | 25:5519377 | 10189 | 25:5520929 | 10189 | 25:5520954 | 10189 | 25:5523535 |
| 10189 | 25:5524957 | 10189 | 25:5527480 | 10189 | 25:5527963 | 10189 | 25:5563162 | 10189 | 25:5586659 |
| 10189 | 25:5591911 | 10190 | 25:5595291 | 10191 | 25:5986235 | 10192 | 25:6069855 | 10194 | 25:6361028 |
| 10197 | 25:7238527 | 10197 | 25:7238686 | 10197 | 25:7239004 | 10203 | 25:8519593 | 10203 | 25:8697220 |
| 10203 | 25:8699100 | 10204 | 25:8709323 | 10204 | 25:8764411 | 10204 | 25:8773984 | 10205 | 25:8963960 |
| 10206 | 25:9320778 | 10206 | 25:9426676 | 10209 | 25:10079477 | 10210 | 25:10133416 | 10213 | 25:10823773 |
| 10213 | 25:10973032 | 10215 | 25:11195704 | 10215 | 25:11212796 | 10219 | 25:12044897 | 10222 | 25:12552168 |
| 10223 | 25:12921926 | 10223 | 25:12926787 | 10224 | 25:13336640 | 10225 | 25:13422767 | 10226 | 25:13560729 |
| 10226 | 25:13702632 | 10227 | 25:14050888 | 10230 | 25:14538936 | 10231 | 25:14817768 | 10231 | 25:14858436 |
| 10232 | 25:14961033 | 10233 | 25:15333545 | 10236 | 25:15932671 | 10241 | 25:16848884 | 10243 | 25:17233999 |
| 10243 | 25:17237242 | 10245 | 25:17765386 | 10258 | 25:21144052 | 10258 | 25:21184609 | 10261 | 25:21797730 |
| 10262 | 25:22007781 | 10263 | 25:22296079 | 10263 | 25:22329885 | 10265 | 25:22669357 | 10266 | 25:22938890 |
| 10268 | 25:23411155 | 10268 | 25:23449496 | 10268 | 25:23538270 | 10270 | 25:23977366 | 10270 | 25:23979613 |
| 10271 | 25:24161354 | 10272 | 25:24271708 | 10274 | 25:24641425 | 10274 | 25:24667454 | 10281 | 25:25961592 |
| 10281 | 25:26009553 | 10283 | 25:26491942 | 10285 | 25:26753501 | 10287 | 25:27390789 | 10292 | 25:27781936 |
| 10299 | 25:29636939 | 10300 | 25:29838430 | 10300 | 25:29838448 | 10301 | 25:29839578 | 10301 | 25:29844263 |
| 10302 | 25:30146443 | 10306 | 25:31190020 | 10307 | 25:31229333 | 10307 | 25:31297977 | 10309 | 25:31607175 |
| 10313 | 25:32422851 | 10313 | 25:32423232 | 10314 | 25:32564396 | 10317 | 25:33134898 | 10318 | 25:33336358 |
| 10320 | 25:33763001 | 10321 | 25:34018085 | 10323 | 25:34358250 | 10326 | 25:34863162 | 10327 | 25:34993980 |
| 10331 | 25:35712925 | 10333 | 25:35998185 | 10334 | 25:36199960 | 10338 | 25:36479714 | 10345 | 25:37601488 |
| 10354 | 25:39461505 | 10355 | 25:39500543 | 10356 | 25:39824171 | 10356 | 25:39824495 | 10358 | 25:40196620 |
| 10361 | 25:40615618 | 10367 | 25:41358682 | 10373 | 26:1198222 | 10373 | 26:1198334 | 10373 | 26:1252329 |
| 10375 | 26:1854681 | 10376 | 26:2064954 | 10377 | 26:2329446 | 10377 | 26:2473264 | 10378 | 26:2489945 |
| 10378 | 26:2571028 | 10381 | 26:3140966 | 10383 | 26:3713174 | 10384 | 26:3717033 | 10389 | 26:4954983 |
| 10389 | 26:5073658 | 10390 | 26:5545287 | 10395 | 26:7011510 | 10395 | 26:7022453 | 10398 | 26:7885953 |
| 10399 | 26:8061770 | 10399 | 26:8088819 | 10399 | 26:8089138 | 10401 | 26:8426892 | 10401 | 26:8486858 |
| 10402 | 26:8674404 | 10404 | 26:9248331 | 10409 | 26:10405639 | 10410 | 26:10640932 | 10413 | 26:11551549 |
| 10418 | 26:12736512 | 10418 | 26:12824626 | 10418 | 26:12837527 | 10419 | 26:12927334 | 10420 | 26:13161640 |
| 10420 | 26:13230527 | 10421 | 26:13230553 | 10421 | 26:13230674 | 10421 | 26:13230708 | 10421 | 26:13230970 |
| 10422 | 26:13354058 | 10424 | 26:14098514 | 0428 | 26:15245070 | 10433 | 26:16547356 | 10433 | 26:16723176 |
| 10437 | 26:17376800 | 10439 | 26:17759010 | 10442 | 26:18446389 | 10444 | 26:18949002 | 10447 | 26:19421103 |
| 10448 | 26:19560868 | 10451 | 26:20617024 | 10451 | 26:20829028 | 10454 | 26:21273598 | 10454 | 26:21380205 |
| 10460 | 26:23188643 | 10463 | 26:24162535 | 10465 | 26:24594195 | 10470 | 26:25362443 | 10474 | 26:25786538 |
| 10476 | 26:26139139 | 10478 | 26:26718349 | 0478 | 26:26723790 | 10479 | 26:26900204 | 10479 | 26:26928555 |
| 10480 | 26:27118119 | 10480 | 26:27128850 | 10480 | 26:27130646 | 10483 | 26:27876991 | 10484 | 26:28054644 |
| 10484 | 26:28086960 | 10488 | 26:29151186 | 10489 | 26:29673193 | 10490 | 26:29804085 | 10496 | 26:31379196 |
| 10503 | 26:33062542 | 10509 | 26:34211364 | 10509 | 26:34259597 | 10510 | 26:34593375 | 10513 | 26:35102465 |
| 10514 | 26:35442752 | 10516 | 26:35826945 | 10518 | 26:36410448 | 10519 | 26:36629017 | 10524 | 26:38084778 |
| 10525 | 26:38264488 | 10525 | 26:38330297 | 10533 | 26:40288755 | 10541 | 26:42332291 | 10543 | 26:42682604 |
| 10545 | 26:43279886 | 10549 | 26:44209268 | 10554 | 26:45319249 | 10556 | 26:45612666 | 10557 | 26:45867614 |
| 10560 | 26:46499606 | 10562 | 26:46841840 | 10566 | 26:47841265 | 10567 | 26:47878623 | 10567 | 26:48135137 |
| 10568 | 26:48274013 | 10571 | 26:48791917 | 10572 | 26:49108450 | 10575 | 26:49581277 | 10577 | 26:50021533 |
| 10577 | 26:50046769 | 10577 | 26:50172060 | 10578 | 26:50483114 | 10581 | 26:50882810 | 10581 | 26:51112488 |
| 10583 | 26:51406962 | 10583 | 26:51407053 | 10583 | 26:51487956 | 10583 | 26:51520196 | 10591 | 27:1670150 |
| 10595 | 27:2495166 | 10601 | 27:4056109 | 10601 | 27:4058537 | 10602 | 27:4060363 | 10602 | 27:4060531 |
| 10602 | 27:4060533 | 10608 | 27:5431758 | 10616 | 27:7970865 | 10620 | 27:8914781 | 10621 | 27:8978523 |
| 10626 | 27:9933403 | 10627 | 27:9948916 | 10632 | 27:11708333 | 10633 | 27:11961138 | 10634 | 27:12169807 |
| 10636 | 27:12847147 | 10638 | 27:13313622 | 10639 | 27:13498868 | 10639 | 27:13539506 | 10640 | 27:13604776 |
| 10640 | 27:13623320 | 10640 | 27:13722354 | 10641 | 27:13774707 | 10641 | 27:13894721 | 10642 | 27:13894736 |
| 10642 | 27:13895111 | 10642 | 27:13895260 | 10642 | 27:13895824 | 10642 | 27:13895841 | 10642 | 27:13896100 |
| 10642 | 27:13896107 | 10642 | 27:13896340 | 10642 | 27:13896382 | 10642 | 27:13896475 | 10643 | 27:13896495 |
| 10643 | 27:13896544 | 10643 | 27:13896557 | 10643 | 27:13896621 | 10643 | 27:13896676 | 10643 | 27:13897078 |
| 10643 | 27:13897184 | 10643 | 27:13897225 | 10643 | 27:13898435 | 10643 | 27:13898864 | 10644 | 27:13899961 |
| 10644 | 27:13900283 | 10644 | 27:13900709 | 10644 | 27:13900927 | 10644 | 27:13900986 | 10644 | 27:13902739 |
| 10644 | 27:13903517 | 10644 | 27:13903525 | 10644 | 27:13903671 | 10644 | 27:13904229 | 10645 | 27:13904269 |
| 10647 | 27:14467858 | 10648 | 27:14760458 | 10648 | 27:14801389 | 10649 | 27:15058579 | 10651 | 27:15382547 |
| 10654 | 27:15918980 | 10654 | 27:16000987 | 10655 | 27:16074052 | 10658 | 27:16407816 | 10658 | 27:16464527 |
| 10662 | 27:17348090 | 10663 | 27:17555135 | 10669 | 27:19049000 | 10669 | 27:19103942 | 10670 | 27:19175688 |
| 10670 | 27:19178944 | 10671 | 27:19583156 | 10677 | 27:20734233 | 10681 | 27:21907032 | 10685 | 27:23015129 |
| 10685 | 27:23015519 | 10686 | 27:23162886 | 10691 | 27:24155175 | 10694 | 27:24823413 | 10695 | 27:24889870 |
| 10695 | 27:25111470 | 10698 | 27:25597037 | 10698 | 27:25632047 | 10701 | 27:26458109 | 10702 | 27:26491670 |
| 10705 | 27:27075217 | 10705 | 27:27089901 | 10708 | 27:27645675 | 10710 | 27:28160084 | 10711 | 27:28613312 |
| 10712 | 27:28845373 | 10713 | 27:29137179 | 10714 | 27:29174979 | 10717 | 27:29722415 | 10719 | 27:30336321 |
| 10719 | 27:30352172 | 10721 | 27:30751847 | 10721 | 27:30799722 | 10725 | 27:31405475 | 10726 | 27:31691203 |
| 10727 | 27:31936569 | 10727 | 27:31937059 | 10727 | 27:31937078 | 10728 | 27:31938645 | 10728 | 27:31939482 |
| 10728 | 27:31940571 | 10733 | 27:32941799 | 10733 | 27:32957490 | 10734 | 27:33063853 | 10738 | 27:34082682 |
| 10742 | 27:35043316 | 10746 | 27:35480823 | 10746 | 27:35485331 | 10746 | 27:35488554 | 10746 | 27:35489257 |
| 10746 | 27:35506982 | 10747 | 27:35556513 | 10747 | 27:35570251 | 10747 | 27:35709604 | 10748 | 27:35789498 |
| 10748 | 27:35863653 | 10749 | 27:35889925 | 10750 | 27:36291522 | 10751 | 27:36452785 | 10752 | 27:36735236 |
| 10755 | 27:37288845 | 10755 | 27:37353731 | 10756 | 27:37379099 | 10760 | 27:38144204 | 10763 | 27:38793276 |
| 10763 | 27:38844060 | 10766 | 27:39447790 | 10768 | 27:40218010 | 10778 | 27:42642792 | 10779 | 27:43019477 |
| 10780 | 27:43086918 | 10780 | 27:43182706 | 10781 | 27:43500535 | 10782 | 27:43689399 | 10785 | 27:44157299 |
| 10786 | 27:44229484 | 10786 | 27:44268404 | 10790 | 27:45312149 | 10794 | 28:1157605 | 10796 | 28:1342853 |
| 10799 | 28:2055151 | 10800 | 28:2304836 | 10802 | 28:3006891 | 10802 | 28:3047863 | 10803 | 28:3081340 |
| 10803 | 28:3085107 | 10807 | 28:5126492 | 10808 | 28:5287434 | 10809 | 28:5566930 | 10812 | 28:5966745 |
| 10819 | 28:7882083 | 10825 | 28:9096925 | 10827 | 28:9589074 | 10835 | 28:12204786 | 10835 | 28:12216204 |
| 10836 | 28:12230473 | 10836 | 28:12253261 | 10836 | 28:12259647 | 10836 | 28:12260824 | 10836 | 28:12274548 |
| 10836 | 28:12276973 | 10836 | 28:12280112 | 10836 | 28:12281177 | 10837 | 28:12285449 | 10837 | 28:12340669 |
| 10838 | 28:12499169 | 10839 | 28:12767375 | 10842 | 28:13530571 | 10842 | 28:13632810 | 10843 | 28:14061176 |
| 10845 | 28:14464098 | 10846 | 28:14523784 | 10853 | 28:16147418 | 10858 | 28:17255203 | 10858 | 28:17266697 |
| 10859 | 28:17297973 | 10859 | 28:17348454 | 10859 | 28:17452718 | 10860 | 28:17453580 | 10860 | 28:17454068 |
| 10860 | 28:17454138 | 10860 | 28:17454593 | 10860 | 28:17454620 | 10860 | 28:17536228 | 10861 | 28:17642513 |
| 10861 | 28:17917993 | 10867 | 28:19218143 | 10870 | 28:20070339 | 10872 | 28:20595474 | 10877 | 28:22093298 |
| 10882 | 28:23296337 | 10884 | 28:23859369 | 10885 | 28:24072970 | 10886 | 28:24222637 | 10893 | 28:25714994 |
| 10898 | 28:26523238 | 10901 | 28:27080357 | 10903 | 28:27474956 | 10904 | 28:27902972 | 10904 | 28:27904071 |
| 10907 | 28:28055097 | 10908 | 28:28119648 | 10911 | 28:28723390 | 10912 | 28:28968643 | 10914 | 28:29407005 |
| 10919 | 28:30842659 | 10920 | 28:31044910 | 10922 | 28:31459804 | 10924 | 28:31905889 | 10925 | 28:32096800 |
| 10927 | 28:32495037 | 10928 | 28:32698642 | 10929 | 28:32777537 | 10929 | 28:32777974 | 10929 | 28:32780910 |
| 10929 | 28:32792000 | 10929 | 28:32793533 | 10929 | 28:32795812 | 10929 | 28:32796515 | 10929 | 28:32801458 |
| 10930 | 28:32803767 | 10930 | 28:32808502 | 10936 | 28:34084210 | 10941 | 28:34925017 | 10941 | 28:34933980 |
| 10943 | 28:35473082 | 10945 | 28:35713729 | 10946 | 28:36047723 | 10946 | 28:36049682 | 10946 | 28:36059895 |
| 10949 | 28:36735400 | 10952 | 28:37458115 | 10952 | 28:37571711 | 10954 | 28:37918674 | 10955 | 28:38086633 |
| 10956 | 28:38273052 | 10956 | 28:38338458 | 10956 | 28:38411536 | 10958 | 28:38715969 | 10959 | 28:39018331 |
| 10962 | 28:39556352 | 10965 | 28:40413981 | 10973 | 28:40820229 | 10973 | 28:40945850 | 10977 | 28:41644978 |
| 10984 | 28:42940481 | 10986 | 28:43165757 | 10996 | 28:45127262 | 10996 | 28:45146124 | 10997 | 28:45229830 |
| 11003 | 29:1136794 | 11004 | 29:1245711 | 11004 | 29:1296904 | 11007 | 29:1876223 | 11009 | 29:2287359 |
| 11009 | 29:2305683 | 11009 | 29:2369666 | 11010 | 29:2449190 | 11010 | 29:2457832 | 11010 | 29:2458133 |
| 11010 | 29:2466616 | 11011 | 29:2475067 | 11011 | 29:2485044 | 11011 | 29:2491210 | 11012 | 29:2791675 |
| 11012 | 29:2792329 | 11013 | 29:2922954 | 11014 | 29:3034446 | 11014 | 29:3054628 | 11015 | 29:3415853 |
| 11016 | 29:3672128 | 11018 | 29:4149425 | 11021 | 29:4771466 | 11021 | 29:4838513 | 11021 | 29:4985399 |
| 11026 | 29:6292926 | 11027 | 29:6759181 | 11030 | 29:7614767 | 11031 | 29:7747910 | 11032 | 29:8183109 |
| 11033 | 29:8211095 | 11034 | 29:8657356 | 11034 | 29:8692066 | 11041 | 29:10237310 | 11042 | 29:10361704 |
| 11043 | 29:10770679 | 11045 | 29:11203337 | 11049 | 29:11981461 | 11053 | 29:12780503 | 11054 | 29:13287093 |
| 11056 | 29:13704499 | 11056 | 29:13711224 | 11058 | 29:14222266 | 11060 | 29:14863923 | 11061 | 29:15023531 |
| 11061 | 29:15136113 | 11063 | 29:15491770 | 11063 | 29:15518673 | 11063 | 29:15577523 | 11063 | 29:15628215 |
| 11063 | 29:15685760 | 11064 | 29:15764983 | 11065 | 29:16007380 | 11065 | 29:16061265 | 11074 | 29:19039096 |
| 11076 | 29:19634491 | 11078 | 29:20300393 | 11079 | 29:20361512 | 11079 | 29:20457751 | 11080 | 29:20716777 |
| 11081 | 29:20812973 | 11081 | 29:20825149 | 11081 | 29:20965169 | 11082 | 29:21317409 | 11082 | 29:21320200 |
| 11082 | 29:21330819 | 11088 | 29:22764191 | 11089 | 29:23102280 | 11092 | 29:23595059 | 11097 | 29:24932130 |
| 11097 | 29:24968087 | 11106 | 29:27477867 | 11107 | 29:27723748 | 11109 | 29:28108791 | 111110 | 29:28257705 |
| 11118 | 29:29705618 | 11124 | 29:30967685 | 11125 | 29:31273255 | 11126 | 29:31338655 | 11136 | 29:33741610 |
| 11136 | 29:33751577 | 11137 | 29:33898078 | 11139 | 29:34412457 | 11140 | 29:34469913 | 11140 | 29:34497844 |
| 11143 | 29:35142257 | 11143 | 29:35191995 | 11147 | 29:35837500 | 11147 | 29:35923286 | 11148 | 29:36087955 |
| 11148 | 29:36106402 | 11150 | 29:36396078 | 11153 | 29:37001499 | 11153 | 29:37010699 | 11155 | 29:37383025 |
| 11156 | 29:37754439 | 11157 | 29:37797892 | 11159 | 29:39272372 | 11160 | 29:39663800 | 11164 | 29:40480123 |
| 11167 | 29:40880222 | 11168 | 29:41078094 | 11169 | 29:41274073 | 11170 | 29:41379850 | 11170 | 29:41422028 |
| 11170 | 29:41439204 | 11170 | 29:41511246 | 11170 | 29:41535408 | 11175 | 29:42380020 | 11180 | 29:43370086 |
| 11185 | 29:44022611 | 11185 | 29:44090714 | 11185 | 29:44103946 | 11185 | 29:44111412 | 11188 | 29:44504806 |
| 11193 | 29:45854845 | 11193 | 29:45910725 | 11197 | 29:46622198 | 11200 | 29:47401443 | 11202 | 29:47889180 |
| 11203 | 29:47969493 | 11206 | 29:48682297 | 11207 | 29:48937511 | 11207 | 29:48937944 | 11207 | 29:48939020 |
| 11207 | 29:49019691 | 11207 | 29:49027895 | 11208 | 29:49027958 | 11208 | 29:49030254 | 11208 | 29:49031741 |
| 11208 | 29:49031778 | 11208 | 29:49032495 | 11208 | 29:49032522 | 11208 | 29:49034373 | 11208 | 29:49034762 |
| 11208 | 29:49036958 | 11208 | 29:49042618 | 11209 | 29:49047332 | 11209 | 29:49047368 | 11209 | 29:49100292 |
| 11209 | 29:49200650 | 11209 | 29:49201144 | 11214 | 29:50487594 | 11215 | 29:50487813 | 11215 | 29:50490270 |
| 11215 | 29:50531220; | | | | | | | | |

and
the sites of the second marker panel are shown in the following table:

| NO. | Position | NO. | Position | NO. | Position | NO. | Position | NO. | Position |
|---|---|---|---|---|---|---|---|---|---|
| 23 | 1: 1010056 | 74 | 1: 1706307 | 78 | 1: 1817641 | 79 | 1: 1821203 | 81 | 1: 1836176 |
| 84 | 1: 1886288 | 87 | 1: 1979359 | 88 | 1: 1997766 | 91 | 1: 2032673 | 109 | 1: 2304587 |
| 112 | 1: 2390062 | 117 | 1: 2749113 | 125 | 1: 3018314 | 138 | 1: 3210484 | 143 | 1: 3282090 |
| 153 | 1: 3479131 | 158 | 1: 3520298 | 174 | 1: 3713849 | 175 | 1: 3716490 | 177 | 1: 3718949 |
| 178 | 1: 3720622 | 179 | 1: 3723615 | 192 | 1: 4021054 | 194 | 1: 4058825 | 197 | 1: 4081869 |
| 200 | 1: 4127064 | 201 | 1: 4130636 | 221 | 1: 4542229 | 245 | 1: 5107763 | 252 | 1: 5359288 |
| 274 | 1: 5770588 | 312 | 1: 6612693 | 320 | 1: 6739866 | 328 | 1: 6816263 | 329 | 1: 6827691 |
| 331 | 1: 6856676 | 333 | 1: 6963461 | 335 | 1: 7027764 | 342 | 1: 7064962 | 349 | 1: 7173547 |
| 351 | 1: 7269335 | 356 | 1: 7378233 | 362 | 1: 7438240 | 363 | 1: 7441167 | 368 | 1: 7637339 |
| 391 | 1: 8312182 | 395 | 1: 8473407 | 406 | 1: 8738949 | 420 | 1: 9069741 | 422 | 1: 9092818 |
| 426 | 1: 9259731 | 431 | 1: 9403159 | 432 | 1: 9442145 | 433 | 1: 9442252 | 434 | 1: 9442256 |
| 435 | 1: 9442802 | 436 | 1: 9467212 | 437 | 1: 9467322 | 439 | 1: 9505564 | 441 | 1: 9525931 |
| 449 | 1: 9675561 | 450 | 1: 9679977 | 451 | 1: 9699218 | 456 | 1: 9843069 | 460 | 1: 9929401 |
| 468 | 1: 10048554 | 472 | 1: 10225421 | 473 | 1: 10225456 | 474 | 1: 10231200 | 476 | 1: 10264906 |
| 477 | 1: 10308984 | 479 | 1: 10360268 | 487 | 1: 10501157 | 490 | 1: 10523191 | 491 | 1: 10537240 |
| 494 | 1: 10563513 | 495 | 1: 10565732 | 498 | 1: 10605121 | 502 | 1: 10771543 | 503 | 1: 10778102 |
| 505 | 1: 10809430 | 506 | 1: 10823521 | 520 | 1: 11223644 | 531 | 1: 11472343 | 535 | 1: 11550314 |
| 544 | 1: 12010431 | 555 | 1: 12378491 | 556 | 1: 12378514 | 566 | 1: 12644235 | 572 | 1: 12803499 |
| 573 | 1: 12803538 | 574 | 1: 12803609 | 584 | 1: 13280975 | 585 | 1: 13288892 | 587 | 1: 13292022 |
| 588 | 1: 13297157 | 591 | 1: 13398597 | 618 | 1: 13725800 | 645 | 1: 14655035 | 648 | 1: 14720755 |
| 668 | 1: 15201042 | 669 | 1: 15201103 | 670 | 1: 15201137 | 695 | 1: 15982024 | 702 | 1: 16155554 |
| 703 | 1: 16155577 | 704 | 1: 16155627 | 705 | 1: 16155668 | 714 | 1: 16447334 | 723 | 1: 16654742 |
| 741 | 1: 17350471 | 748 | 1: 17481344 | 749 | 1: 17481363 | 753 | 1: 17580720 | 774 | 1: 18459894 |
| 799 | 1: 19402363 | 800 | 1: 19409456 | 804 | 1: 19493132 | 814 | 1: 19676162 | 819 | 1: 19929109 |
| 833 | 1: 20279571 | 837 | 1: 20407748 | 840 | 1: 20480157 | 845 | 1: 20570353 | 847 | 1: 20621543 |
| 857 | 1: 20823430 | 860 | 1: 20921794 | 861 | 1: 20974759 | 863 | 1: 21005976 | 868 | 1: 21167645 |
| 872 | 1: 21346196 | 883 | 1: 21646634 | 890 | 1: 21783397 | 896 | 1: 21921230 | 902 | 1: 22076454 |
| 925 | 1: 22688749 | 933 | 1: 22949333 | 947 | 1: 23444110 | 952 | 1: 23709011 | 953 | 1: 23709064 |
| 990 | 1: 24753266 | 993 | 1: 24782983 | 1010 | 1: 25234761 | 1011 | 1: 25234832 | 1023 | 1: 25497647 |
| 1056 | 1: 26533932 | 1058 | 1: 26552621 | 1061 | 1: 26559576 | 1063 | 1: 26611592 | 1067 | 1: 26755277 |
| 1070 | 1: 26880560 | 1072 | 1: 26882959 | 1077 | 1: 26969554 | 1082 | 1: 27132369 | 1084 | 1: 27171038 |
| 1092 | 1: 27401121 | 1134 | 1: 29068209 | 1144 | 1: 29340605 | 1147 | 1: 29403350 | 1151 | 1: 29458506 |
| 1158 | 1: 29712048 | 1165 | 1: 29884320 | 1170 | 1: 29949770 | 1180 | 1: 30194782 | 1201 | 1: 30897798 |
| 1219 | 1: 31546632 | 1227 | 1: 31736868 | 1249 | 1: 32659120 | 1262 | 1: 33136711 | 1265 | 1: 33292309 |
| 1270 | 1: 33496557 | 1272 | 1: 33533012 | 1289 | 1: 33884832 | 1300 | 1: 34174940 | 1309 | 1: 34602877 |
| 1311 | 1: 34663000 | 1313 | 1: 34664612 | 1317 | 1: 34824821 | 1318 | 1: 34824930 | 1321 | 1: 34924925 |
| 1324 | 1: 35000632 | 1331 | 1: 35082343 | 1344 | 1: 35345367 | 1349 | 1: 35407003 | 1362 | 1: 35733756 |
| 1370 | 1: 36018590 | 1372 | 1: 36111561 | 1379 | 1: 36303126 | 1389 | 1: 36436645 | 1392 | 1: 36459421 |
| 1393 | 1: 36459510 | 1402 | 1: 36697707 | 1408 | 1: 36828697 | 1414 | 1: 37032363 | 1415 | 1: 37032440 |
| 1426 | 1: 37411283 | 1428 | 1: 37442242 | 1434 | 1: 37638872 | 1438 | 1: 37686047 | 1440 | 1: 37725772 |
| 1450 | 1: 37956983 | 1453 | 1: 38013891 | 1468 | 1: 38540153 | 1469 | 1: 38599466 | 1476 | 1: 38720880 |
| 1494 | 1: 39249574 | 1505 | 1: 39697516 | 1534 | 1: 40506922 | 1538 | 1: 40629765 | 1548 | 1: 40789010 |
| 1573 | 1: 41686185 | 1579 | 1: 41784113 | 1580 | 1: 41788052 | 1581 | 1: 41795018 | 1582 | 1: 41795589 |
| 1583 | 1: 41796750 | 1584 | 1: 41797989 | 1586 | 1: 41805753 | 1587 | 1: 41835927 | 1588 | 1: 41836033 |
| 1590 | 1: 41886688 | 1600 | 1: 42107409 | 1601 | 1: 42131473 | 1604 | 1: 42159211 | 1605 | 1: 42170788 |
| 1609 | 1: 42224416 | 1611 | 1: 42265122 | 1612 | 1: 42285326 | 1613 | 1: 42293500 | 1619 | 1: 42519517 |
| 1633 | 1: 42917734 | 1640 | 1: 43141355 | 1643 | 1: 43211702 | 1645 | 1: 43225435 | 1647 | 1: 43292695 |
| 1652 | 1: 43443436 | 1656 | 1: 43496586 | 1668 | 1: 43756861 | 1670 | 1: 43772333 | 1672 | 1: 43787631 |
| 1684 | 1: 44030562 | 1686 | 1: 44124010 | 1688 | 1: 44137020 | 1690 | 1: 44236960 | 1700 | 1: 44393495 |
| 1701 | 1: 44422846 | 1708 | 1: 44623944 | 1712 | 1: 44766328 | 1714 | 1: 44820415 | 1715 | 1: 44820480 |
| 1716 | 1: 44820576 | 1717 | 1: 44847495 | 1732 | 1: 44947997 | 1733 | 1: 44956505 | 1734 | 1: 44959367 |
| 1735 | 1: 44960388 | 1737 | 1: 44995524 | 1738 | 1: 44998217 | 1740 | 1: 45030214 | 1741 | 1: 45036157 |
| 1744 | 1: 45061133 | 1748 | 1: 45196717 | 1750 | 1: 45216991 | 1759 | 1: 45365527 | 1760 | 1: 45372759 |
| 1784 | 1: 46185873 | 1793 | 1: 46488716 | 1821 | 1: 47439819 | 1822 | 1: 47457097 | 1823 | 1: 47465147 |
| 1829 | 1: 47631393 | 1852 | 1: 48538887 | 1853 | 1: 48538897 | 1866 | 1: 48823158 | 1893 | 1: 49844520 |
| 1907 | 1: 50327235 | 1913 | 1: 50531202 | 1916 | 1: 50627944 | 1917 | 1: 50631061 | 1918 | 1: 50631100 |
| 1919 | 1: 50637655 | 1924 | 1: 50846942 | 1925 | 1: 50856429 | 1926 | 1: 50874802 | 1928 | 1: 50934316 |
| 1931 | 1: 50978540 | 1934 | 1: 51048823 | 1945 | 1: 51501180 | 1958 | 1: 51956854 | 1960 | 1: 52001408 |
| 1974 | 1: 52418784 | 1975 | 1: 52420721 | 1977 | 1: 52468623 | 1979 | 1: 52499614 | 1980 | 1: 52499623 |
| 1982 | 1: 52592314 | 1983 | 1: 52616071 | 1984 | 1: 52639898 | 1989 | 1: 52845116 | 1999 | 1: 53149622 |
| 2008 | 1: 53293355 | 2011 | 1: 53338531 | 2015 | 1: 53534215 | 2034 | 1: 53685551 | 2036 | 1: 53685641 |
| 2037 | 1: 53685662 | 2038 | 1: 53685710 | 2043 | 1: 53760822 | 2049 | 1: 53972219 | 2055 | 1: 54092473 |
| 2056 | 1: 54092637 | 2057 | 1: 54092679 | 2058 | 1: 54093927 | 2059 | 1: 54101343 | 2068 | 1: 54343019 |
| 2076 | 1: 54460688 | 2088 | 1: 54757168 | 2089 | 1: 54766036 | 2096 | 1: 55026386 | 2097 | 1: 55026433 |
| 2108 | 1: 55421744 | 2110 | 1: 55482490 | 2121 | 1: 55927365 | 2122 | 1: 55964487 | 2123 | 1: 55970922 |
| 2132 | 1: 56253478 | 2138 | 1: 56370458 | 2142 | 1: 56472558 | 2145 | 1: 56498727 | 2147 | 1: 56540591 |
| 2154 | 1: 56759097 | 2162 | 1: 56937696 | 2165 | 1: 56975870 | 2170 | 1: 57137491 | 2173 | 1: 57217658 |
| 2174 | 1: 57217807 | 2175 | 1: 57251079 | 2176 | 1: 57266134 | 2178 | 1: 57309255 | 2181 | 1: 57398938 |
| 2184 | 1: 57490618 | 2185 | 1: 57560306 | 2197 | 1: 57870387 | 2198 | 1: 57917830 | 2203 | 1: 58030359 |
| 2205 | 1: 58075432 | 2206 | 1: 58075463 | 2207 | 1: 58075499 | 2208 | 1: 58075521 | 2209 | 1: 58078354 |
| 2210 | 1: 58078466 | 2211 | 1: 58097510 | 2221 | 1: 58305398 | 2222 | 1: 58305455 | 2223 | 1: 58315322 |
| 2224 | 1: 58315443 | 2225 | 1: 58315990 | 2231 | 1: 58540410 | 2232 | 1: 58618667 | 2236 | 1: 58750747 |
| 2237 | 1: 58773333 | 2240 | 1: 58818870 | 2250 | 1: 59034012 | 2252 | 1: 59104762 | 2258 | 1: 59200011 |
| 2259 | 1: 59203569 | 2263 | 1: 59371385 | 2270 | 1: 59656138 | 2279 | 1: 59830679 | 2293 | 1: 60123244 |
| 2318 | 1: 60784872 | 2325 | 1: 60918912 | 2330 | 1: 61097118 | 2333 | 1: 61128757 | 2336 | 1: 61160140 |
| 2369 | 1: 62066946 | 2370 | 1: 62092465 | 2386 | 1: 62457515 | 2399 | 1: 62621737 | 2402 | 1: 62637571 |
| 2406 | 1: 62817788 | 2407 | 1: 62817866 | 2428 | 1: 63139698 | 2438 | 1: 63427104 | 2440 | 1: 63476336 |
| 2441 | 1: 63517812 | 2451 | 1: 63851933 | 2456 | 1: 64008039 | 2461 | 1: 64183671 | 2463 | 1: 64200486 |
| 2464 | 1: 64200539 | 2466 | 1: 64262468 | 2477 | 1: 64431800 | 2478 | 1: 64462597 | 2479 | 1: 64462675 |
| 2480 | 1: 64463152 | 2481 | 1: 64463254 | 2482 | 1: 64467138 | 2483 | 1: 64467211 | 2486 | 1: 64505435 |
| 2496 | 1: 64824564 | 2502 | 1: 64940185 | 2509 | 1: 65157725 | 2512 | 1: 65199200 | 2513 | 1: 65216449 |
| 2514 | 1: 65224381 | 2515 | 1: 65228988 | 2516 | 1: 65239311 | 2520 | 1: 65377133 | 2545 | 1: 66100053 |
| 2547 | 1: 66136711 | 2557 | 1: 66502772 | 2558 | 1: 66511698 | 2559 | 1: 66515809 | 2561 | 1: 66547988 |
| 2569 | 1: 66694865 | 2581 | 1: 67173187 | 2588 | 1: 67320099 | 2590 | 1: 67331681 | 2591 | 1: 67411736 |
| 2593 | 1: 67432038 | 2594 | 1: 67439085 | 2596 | 1: 67473778 | 2604 | 1: 67760033 | 2608 | 1: 67861511 |
| 2618 | 1: 68238481 | 2619 | 1: 68259739 | 2623 | 1: 68332459 | 2627 | 1: 68449381 | 2628 | 1: 68488305 |
| 2630 | 1: 68501205 | 2631 | 1: 68517809 | 2635 | 1: 68649543 | 2637 | 1: 68680831 | 2638 | 1: 68682422 |
| 2639 | 1: 68682461 | 2640 | 1: 68690274 | 2644 | 1: 68784544 | 2646 | 1: 68792494 | 2647 | 1: 68793488 |
| 2648 | 1: 68793540 | 2649 | 1: 68793542 | 2650 | 1: 68793543 | 2651 | 1: 68797693 | 2656 | 1: 68869228 |
| 2663 | 1: 68996534 | 2668 | 1: 69017745 | 2670 | 1: 69042317 | 2671 | 1: 69047274 | 2672 | 1: 69047282 |
| 2673 | 1: 69047320 | 2679 | 1: 69124262 | 2682 | 1: 69203913 | 2684 | 1: 69217319 | 2685 | 1: 69217672 |
| 2698 | 1: 69582986 | 2699 | 1: 69585403 | 2704 | 1: 69764910 | 2712 | 1: 69883156 | 2717 | 1: 69982394 |
| 2720 | 1: 70106968 | 2725 | 1: 70231031 | 2726 | 1: 70231761 | 2727 | 1: 70248171 | 2730 | 1: 70343458 |
| 2732 | 1: 70365636 | 2734 | 1: 70390927 | 2741 | 1: 70466635 | 2742 | 1: 70466719 | 2743 | 1: 70466765 |
| 2744 | 1: 70468358 | 2745 | 1: 70473449 | 2746 | 1: 70473451 | 2747 | 1: 70473452 | 2748 | 1: 70473471 |
| 2749 | 1: 70473493 | 2750 | 1: 70485290 | 2751 | 1: 70485353 | 2753 | 1: 70494055 | 2754 | 1: 70497518 |
| 2755 | 1: 70500089 | 2762 | 1: 70616796 | 2771 | 1: 70783887 | 2772 | 1: 70791872 | 2773 | 1: 70801580 |
| 2790 | 1: 71415748 | 2791 | 1: 71416509 | 2792 | 1: 71416751 | 2793 | 1: 71416881 | 2794 | 1: 71416890 |
| 2803 | 1: 71609698 | 2817 | 1: 72069890 | 2819 | 1: 72081243 | 2820 | 1: 72090597 | 2834 | 1: 72454020 |
| 2836 | 1: 72484009 | 2839 | 1: 72519103 | 2849 | 1: 72679096 | 2850 | 1: 72679109 | 2851 | 1: 72679167 |
| 2854 | 1: 72714931 | 2855 | 1: 72726681 | 2856 | 1: 72732857 | 2859 | 1: 72803314 | 2863 | 1: 72952213 |
| 2864 | 1: 72955756 | 2866 | 1: 73093141 | 2871 | 1: 73187152 | 2872 | 1: 73207902 | 2879 | 1: 73477972 |
| 2883 | 1: 73591733 | 2887 | 1: 73663050 | 2888 | 1: 73671409 | 2890 | 1: 73744599 | 2898 | 1: 73902125 |
| 2902 | 1: 73954764 | 2908 | 1: 74183606 | 2915 | 1: 74460825 | 2917 | 1: 74460955 | 2918 | 1: 74489282 |
| 2919 | 1: 74490359 | 2922 | 1: 74553801 | 2935 | 1: 74976635 | 2957 | 1: 75592224 | 2960 | 1: 75682742 |
| 2967 | 1: 75798251 | 2968 | 1: 75798252 | 2969 | 1: 75798317 | 2972 | 1: 75852855 | 2980 | 1: 76051714 |
| 3013 | 1: 77045567 | 3020 | 1: 77255234 | 3029 | 1: 77397999 | 3032 | 1: 77443583 | 3033 | 1: 77469880 |
| 3036 | 1: 77593114 | 3038 | 1: 77616604 | 3040 | 1: 77652308 | 3044 | 1: 77698757 | 3048 | 1: 77727505 |
| 3054 | 1: 77958907 | 3074 | 1: 78375438 | 3077 | 1: 78445734 | 3082 | 1: 78556250 | 3083 | 1: 78566682 |
| 3093 | 1: 78911959 | 3095 | 1: 78989138 | 3098 | 1: 79082187 | 3101 | 1: 79138960 | 3102 | 1: 79169504 |
| 3105 | 1: 79213373 | 3107 | 1: 79242860 | 3110 | 1: 79298445 | 3114 | 1: 79357690 | 3118 | 1: 79485282 |
| 3123 | 1: 79562994 | 3156 | 1: 80023498 | 3161 | 1: 80069552 | 3163 | 1: 80074105 | 3172 | 1: 80391190 |
| 3173 | 1: 80413261 | 3183 | 1: 80685030 | 3192 | 1: 80940339 | 3196 | 1: 81028408 | 3201 | 1: 81051808 |
| 3208 | 1: 81170302 | 3209 | 1: 81183483 | 3215 | 1: 81348584 | 3229 | 1: 81706937 | 3235 | 1: 81878324 |
| 3240 | 1: 82041541 | 3241 | 1: 82041559 | 3242 | 1: 82047704 | 3246 | 1: 82149539 | 3258 | 1: 82473626 |
| 3264 | 1: 82519837 | 3265 | 1: 82541328 | 3266 | 1: 82541450 | 3268 | 1: 82558245 | 3273 | 1: 82643904 |
| 3276 | 1: 82705721 | 3278 | 1: 82758391 | 3284 | 1: 83070275 | 3285 | 1: 83097205 | 3286 | 1: 83133001 |
| 3291 | 1: 83278833 | 3292 | 1: 83284140 | 3293 | 1: 83330542 | 3306 | 1: 83810343 | 3310 | 1: 83974373 |
| 3312 | 1: 84000147 | 3315 | 1: 84047005 | 3316 | 1: 84058598 | 3338 | 1: 85042289 | 3344 | 1: 85087002 |
| 3346 | 1: 85130568 | 3368 | 1: 85947378 | 3369 | 1: 85948692 | 3371 | 1: 85976228 | 3374 | 1: 86122181 |
| 3375 | 1: 86146073 | 3376 | 1: 86156408 | 3377 | 1: 86160591 | 3378 | 1: 86177464 | 3379 | 1: 86185749 |
| 3380 | 1: 86186427 | 3394 | 1: 86631754 | 3406 | 1: 86967586 | 3420 | 1: 87399098 | 3421 | 1: 87399338 |
| 3422 | 1: 87414899 | 3428 | 1: 87574019 | 3431 | 1: 87648729 | 3434 | 1: 87752223 | 3441 | 1: 88012422 |
| 3467 | 1: 88872312 | 3468 | 1: 88874634 | 3469 | 1: 88880495 | 3471 | 1: 88896168 | 3472 | 1: 88908213 |
| 3474 | 1: 88987015 | 3476 | 1: 89086745 | 3478 | 1: 89097927 | 3479 | 1: 89098024 | 3482 | 1: 89224144 |
| 3484 | 1: 89300003 | 3486 | 1: 89343474 | 3487 | 1: 89349576 | 3488 | 1: 89383250 | 3492 | 1: 89498954 |
| 3494 | 1: 89539395 | 3495 | 1: 89539442 | 3508 | 1: 89796658 | 3509 | 1: 89799413 | 3522 | 1: 90043900 |
| 3524 | 1: 90075379 | 3550 | 1: 90588788 | 3552 | 1: 90615269 | 3554 | 1: 90648889 | 3576 | 1: 91255865 |
| 3577 | 1: 91285295 | 3593 | 1: 91706481 | 3596 | 1: 91737921 | 3597 | 1: 91739554 | 3602 | 1: 91928880 |
| 3609 | 1: 92184004 | 3623 | 1: 92503312 | 3668 | 1: 93883957 | 3670 | 1: 93930496 | 3674 | 1: 94035144 |
| 3675 | 1: 94035267 | 3680 | 1: 94131411 | 3681 | 1: 94134092 | 3699 | 1: 94576910 | 3709 | 1: 94877027 |
| 3729 | 1: 95423137 | 3730 | 1: 95439594 | 3731 | 1: 95455255 | 3733 | 1: 95478530 | 3734 | 1: 95494762 |
| 3745 | 1: 95806271 | 3747 | 1: 95893257 | 3752 | 1: 96002349 | 3754 | 1: 96026129 | 3756 | 1: 96084552 |
| 3757 | 1: 96085021 | 3760 | 1: 96180861 | 3764 | 1: 96218350 | 3765 | 1: 96220152 | 3770 | 1: 96344874 |
| 3779 | 1: 96474332 | 3784 | 1: 96640052 | 3801 | 1: 97152251 | 3819 | 1: 97537514 | 3832 | 1: 97878133 |
| 3843 | 1: 98148099 | 3847 | 1: 98313182 | 3857 | 1: 98673491 | 3867 | 1: 98891260 | 3876 | 1: 99200317 |
| 3882 | 1: 99297524 | 3883 | 1: 99323405 | 3886 | 1: 99418906 | 3890 | 1: 99501232 | 3906 | 1: 99941713 |
| 3914 | 1: 100198782 | 3917 | 1: 100296109 | 3919 | 1: 100321230 | 3927 | 1: 100567937 | 3948 | 1: 101273216 |
| 3969 | 1: 101894473 | 3980 | 1: 102388029 | 3981 | 1: 102388112 | 3988 | 1: 102582187 | 3990 | 1: 102591751 |
| 3993 | 1: 102638499 | 3995 | 1: 102646032 | 3997 | 1: 102698749 | 4004 | 1: 102911722 | 4008 | 1: 103023798 |
| 4037 | 1: 104209003 | 4038 | 1: 104263712 | 4045 | 1: 104522827 | 4053 | 1: 104831357 | 4058 | 1: 104915762 |
| 4060 | 1: 104987991 | 4061 | 1: 105015424 | 4074 | 1: 105367807 | 4079 | 1: 105447199 | 4103 | 1: 106048772 |
| 4108 | 1: 106226124 | 4111 | 1: 106284785 | 4123 | 1: 106497025 | 4144 | 1: 107326886 | 4149 | 1: 107424074 |
| 4153 | 1: 107500490 | 4154 | 1: 107538852 | 4155 | 1: 107544465 | 4156 | 1: 107544536 | 4161 | 1: 107781031 |
| 4164 | 1: 107863018 | 4170 | 1: 108114016 | 4189 | 1: 108515165 | 4191 | 1: 108556182 | 4192 | 1: 108556227 |
| 4203 | 1: 108851719 | 4204 | 1: 108851745 | 4205 | 1: 108853578 | 4206 | 1: 108854832 | 4207 | 1: 108870778 |
| 4208 | 1: 108874776 | 4209 | 1: 108881505 | 4210 | 1: 108884859 | 4211 | 1: 108894708 | 4212 | 1: 108894728 |
| 4213 | 1: 108896221 | 4230 | 1: 109527893 | 4231 | 1: 109527919 | 4245 | 1: 109820651 | 4250 | 1: 109870082 |
| 4266 | 1: 110126279 | 4267 | 1: 110175621 | 4269 | 1: 110196398 | 4273 | 1: 110293441 | 4276 | 1: 110341970 |
| 4277 | 1: 110342054 | 4278 | 1: 110342111 | 4283 | 1: 110476863 | 4288 | 1: 110573226 | 4289 | 1: 110590869 |
| 4292 | 1: 110633655 | 4293 | 1: 110653778 | 4295 | 1: 110665646 | 4296 | 1: 110666740 | 4302 | 1: 110816709 |
| 4309 | 1: 111186619 | 4310 | 1: 111194151 | 4311 | 1: 111199408 | 4312 | 1: 111247418 | 4313 | 1: 111268099 |
| 4325 | 1: 111708777 | 4327 | 1: 111748641 | 4331 | 1: 111775768 | 4332 | 1: 111775805 | 4333 | 1: 111798208 |
| 4345 | 1: 112208772 | 4353 | 1: 112368514 | 4354 | 1: 112398275 | 4355 | 1: 112400081 | 4361 | 1: 112555088 |
| 4362 | 1: 112555094 | 4363 | 1: 112555107 | 4364 | 1: 112555108 | 4365 | 1: 112555300 | 4366 | 1: 112555322 |
| 4367 | 1: 112555340 | 4368 | 1: 112555460 | 4369 | 1: 112555492 | 4370 | 1: 112556677 | 4371 | 1: 112556716 |
| 4372 | 1: 112556776 | 4375 | 1: 112581875 | 4376 | 1: 112581930 | 4377 | 1: 112581975 | 4378 | 1: 112595293 |
| 4400 | 1: 113354543 | 4403 | 1: 113365903 | 4404 | 1: 113369018 | 4405 | 1: 113385595 | 4406 | 1: 113385624 |
| 4407 | 1: 113385658 | 4417 | 1: 113584302 | 4457 | 1: 114467455 | 4460 | 1: 114596976 | 4462 | 1: 114728742 |
| 4465 | 1: 114816068 | 4468 | 1: 114840022 | 4488 | 1: 115324305 | 4490 | 1: 115377104 | 4491 | 1: 115413995 |
| 4496 | 1: 115579666 | 4502 | 1: 115790600 | 4528 | 1: 116754887 | 4532 | 1: 116818074 | 4533 | 1: 116821719 |
| 4535 | 1: 116860895 | 4538 | 1: 116891857 | 4540 | 1: 117003117 | 4546 | 1: 117113278 | 4556 | 1: 117319749 |
| 4563 | 1: 117420923 | 4564 | 1: 117421250 | 4576 | 1: 117738265 | 4578 | 1: 117758868 | 4586 | 1: 117887255 |
| 4587 | 1: 117897860 | 4588 | 1: 117912537 | 4589 | 1: 117935755 | 4591 | 1: 117945817 | 4597 | 1: 118048415 |
| 4598 | 1: 118085158 | 4600 | 1: 118114537 | 4602 | 1: 118133789 | 4605 | 1: 118196612 | 4608 | 1: 118214654 |
| 4609 | 1: 118227302 | 4610 | 1: 118256496 | 4611 | 1: 118257517 | 4612 | 1: 118259606 | 4613 | 1: 118273499 |
| 4614 | 1: 118276261 | 4615 | 1: 118276735 | 4616 | 1: 118277267 | 4617 | 1: 118278387 | 4618 | 1: 118284875 |
| 4619 | 1: 118286166 | 4620 | 1: 118288018 | 4621 | 1: 118290177 | 4622 | 1: 118295131 | 4635 | 1: 118530178 |
| 4636 | 1: 118544527 | 4642 | 1: 118690300 | 4643 | 1: 118690309 | 4652 | 1: 118962324 | 4654 | 1: 118985100 |
| 4665 | 1: 119176913 | 4666 | 1: 119189605 | 4667 | 1: 119210631 | 4672 | 1: 119350100 | 4683 | 1: 119561515 |
| 4684 | 1: 119561537 | 4685 | 1: 119561564 | 4701 | 1: 120064732 | 4712 | 1: 120386879 | 4722 | 1: 120500835 |
| 4726 | 1: 120563845 | 4760 | 1: 121647921 | 4762 | 1: 121707542 | 4771 | 1: 121946641 | 4781 | 1: 122232742 |
| 4785 | 1: 122286938 | 4786 | 1: 122287045 | 4790 | 1: 122500672 | 4794 | 1: 122540031 | 4799 | 1: 122654668 |
| 4801 | 1: 122686036 | 4807 | 1: 122783659 | 4808 | 1: 122784649 | 4810 | 1: 122849903 | 4811 | 1: 122901551 |
| 4814 | 1: 123034294 | 4819 | 1: 123266689 | 4821 | 1: 123270508 | 4827 | 1: 123475726 | 4835 | 1: 123679492 |
| 4836 | 1: 123692405 | 4837 | 1: 123706359 | 4838 | 1: 123709988 | 4839 | 1: 123720581 | 4848 | 1: 124014834 |
| 4854 | 1: 124194081 | 4860 | 1: 124271732 | 4861 | 1: 124271768 | 4862 | 1: 124271786 | 4863 | 1: 124314731 |
| 4864 | 1: 124324829 | 4865 | 1: 124331752 | 4866 | 1: 124343187 | 4870 | 1: 124506907 | 4871 | 1: 124528147 |
| 4878 | 1: 124666898 | 4879 | 1: 124672817 | 4886 | 1: 124887464 | 4891 | 1: 124948606 | 4894 | 1: 124980393 |
| 4895 | 1: 124980562 | 4898 | 1: 125121982 | 4902 | 1: 125269301 | 4904 | 1: 125360929 | 4905 | 1: 125363719 |
| 4907 | 1: 125382225 | 4908 | 1: 125386885 | 4913 | 1: 125521147 | 4919 | 1: 125583210 | 4920 | 1: 125608898 |
| 4922 | 1: 125609041 | 4928 | 1: 125643614 | 4944 | 1: 125851156 | 4946 | 1: 125931085 | 4951 | 1: 126050865 |
| 4953 | 1: 126166588 | 4954 | 1: 126167017 | 4955 | 1: 126188011 | 4962 | 1: 126320901 | 4970 | 1: 126452685 |
| 4979 | 1: 126828386 | 4981 | 1: 126867108 | 4989 | 1: 127247333 | 4997 | 1: 127448104 | 5001 | 1: 127536751 |
| 5004 | 1: 127609705 | 5007 | 1: 127732186 | 5010 | 1: 127791769 | 5011 | 1: 127843966 | 5015 | 1: 127884865 |
| 5020 | 1: 127963540 | 5022 | 1: 128091201 | 5032 | 1: 128267812 | 5037 | 1: 128374395 | 5042 | 1: 128512081 |
| 5073 | 1: 129150388 | 5080 | 1: 129402104 | 5081 | 1: 129418328 | 5089 | 1: 129590492 | 5090 | 1: 129591720 |
| 5091 | 1: 129592529 | 5095 | 1: 129754416 | 5127 | 1: 130632012 | 5128 | 1: 130658736 | 5130 | 1: 130720354 |
| 5131 | 1: 130728433 | 5134 | 1: 130792978 | 5137 | 1: 130920062 | 5140 | 1: 130954555 | 5179 | 1: 131419104 |
| 5222 | 1: 131860577 | 5242 | 1: 132473942 | 5253 | 1: 132876761 | 5264 | 1: 133379843 | 5265 | 1: 133384149 |
| 5266 | 1: 133394080 | 5271 | 1: 133540327 | 5272 | 1: 133591491 | 5289 | 1: 134186323 | 5294 | 1: 134285791 |
| 5298 | 1: 134384145 | 5300 | 1: 134413818 | 5301 | 1: 134413821 | 5306 | 1: 134477738 | 5324 | 1: 134948789 |
| 5325 | 1: 134961346 | 5327 | 1: 134973908 | 5329 | 1: 135015189 | 5331 | 1: 135049866 | 5337 | 1: 135208575 |
| 5341 | 1: 135315701 | 5342 | 1: 135345248 | 5346 | 1: 135410192 | 5347 | 1: 135414049 | 5360 | 1: 135642886 |
| 5361 | 1: 135643955 | 5362 | 1: 135644811 | 5363 | 1: 135653473 | 5366 | 1: 135665914 | 5367 | 1: 135666200 |
| 5368 | 1: 135666237 | 5369 | 1: 135666283 | 5370 | 1: 135670574 | 5371 | 1: 135672004 | 5372 | 1: 135694251 |
| 5374 | 1: 135753894 | 5375 | 1: 135753940 | 5376 | 1: 135753983 | 5377 | 1: 135754044 | 5378 | 1: 135754264 |
| 5379 | 1: 135754300 | 5380 | 1: 135758284 | 5381 | 1: 135758290 | 5382 | 1: 135758332 | 5383 | 1: 135762033 |
| 5384 | 1: 135763704 | 5385 | 1: 135769522 | 5397 | 1: 135880893 | 5399 | 1: 135916803 | 5400 | 1: 135927623 |
| 5401 | 1: 135972509 | 5402 | 1: 135974364 | 5405 | 1: 136033754 | 5406 | 1: 136047835 | 5413 | 1: 136204362 |
| 5415 | 1: 136311162 | 5427 | 1: 136734614 | 5434 | 1: 137049621 | 5438 | 1: 137106364 | 5443 | 1: 137294127 |
| 5472 | 1: 138643152 | 5473 | 1: 138647040 | 5477 | 1: 138728014 | 5478 | 1: 138739021 | 5483 | 1: 138798912 |
| 5489 | 1: 138994500 | 5498 | 1: 139184610 | 5505 | 1: 139411806 | 5508 | 1: 139449113 | 5521 | 1: 139899458 |
| 5536 | 1: 140266541 | 5545 | 1: 140543741 | 5554 | 1: 140754935 | 5555 | 1: 140755072 | 5558 | 1: 140876675 |
| 5575 | 1: 141145402 | 5579 | 1: 141316735 | 5580 | 1: 141319127 | 5581 | 1: 141319994 | 5583 | 1: 141339227 |
| 5584 | 1: 141345591 | 5585 | 1: 141362575 | 5586 | 1: 141364352 | 5587 | 1: 141374294 | 5588 | 1: 141376997 |
| 5590 | 1: 141395382 | 5591 | 1: 141421683 | 5597 | 1: 141511318 | 5601 | 1: 141610105 | 5602 | 1: 141610924 |
| 5604 | 1: 141627871 | 5606 | 1: 141628909 | 5608 | 1: 141649600 | 5610 | 1: 141651362 | 5611 | 1: 141653130 |
| 5612 | 1: 141653134 | 5613 | 1: 141654271 | 5614 | 1: 141654547 | 5615 | 1: 141654551 | 5617 | 1: 141667906 |
| 5622 | 1: 141734030 | 5623 | 1: 141741068 | 5625 | 1: 141823289 | 5633 | 1: 142000532 | 5641 | 1: 142239516 |
| 5643 | 1: 142251697 | 5651 | 1: 142362891 | 5654 | 1: 142393561 | 5657 | 1: 142478032 | 5659 | 1: 142489032 |
| 5667 | 1: 142635123 | 5672 | 1: 142787751 | 5693 | 1: 143128593 | 5700 | 1: 143227118 | 5702 | 1: 143334405 |
| 5706 | 1: 143406113 | 5711 | 1: 143569447 | 5712 | 1: 143579707 | 5713 | 1: 143582830 | 5714 | 1: 143588692 |
| 5715 | 1: 143591196 | 5716 | 1: 143593029 | 5717 | 1: 143594628 | 5726 | 1: 143746215 | 5728 | 1: 143749139 |
| 5729 | 1: 143752954 | 5747 | 1: 144050118 | 5748 | 1: 144050120 | 5757 | 1: 144333701 | 5759 | 1: 144372951 |
| 5763 | 1: 144435905 | 5772 | 1: 144635480 | 5776 | 1: 144757986 | 5780 | 1: 144787067 | 5781 | 1: 144809797 |
| 5786 | 1: 144894365 | 5794 | 1: 145080672 | 5797 | 1: 145128073 | 5799 | 1: 145136789 | 5802 | 1: 145176107 |
| 5804 | 1: 145282436 | 5813 | 1: 145459919 | 5821 | 1: 145623145 | 5822 | 1: 145623795 | 5825 | 1: 145673098 |
| 5826 | 1: 145677709 | 5834 | 1: 145827286 | 5835 | 1: 145830272 | 5837 | 1: 145860541 | 5838 | 1: 145860568 |
| 5839 | 1: 145866834 | 5840 | 1: 145866900 | 5841 | 1: 145873941 | 5842 | 1: 145873979 | 5843 | 1: 145874030 |
| 5848 | 1: 145951072 | 5849 | 1: 145963833 | 5850 | 1: 145968413 | 5852 | 1: 145972525 | 5853 | 1: 145975945 |
| 5854 | 1: 145977236 | 5862 | 1: 146052159 | 5871 | 1: 146351589 | 5874 | 1: 146420270 | 5892 | 1: 146975928 |
| 5894 | 1: 147008926 | 5899 | 1: 147098097 | 5902 | 1: 147122843 | 5918 | 1: 147397092 | 5919 | 1: 147417101 |
| 5921 | 1: 147431978 | 5927 | 1: 147563989 | 5941 | 1: 147846791 | 5946 | 1: 147903408 | 5978 | 1: 148430252 |
| 5979 | 1: 148466864 | 5981 | 1: 148508063 | 5982 | 1: 148508133 | 5984 | 1: 148513206 | 5985 | 1: 148549091 |
| 5994 | 1: 148736098 | 6000 | 1: 148871697 | 6004 | 1: 148964961 | 6010 | 1: 149177892 | 6011 | 1: 149189606 |
| 6014 | 1: 149317573 | 6015 | 1: 149320003 | 6016 | 1: 149323616 | 6017 | 1: 149324955 | 6018 | 1: 149328203 |
| 6019 | 1: 149340128 | 6020 | 1: 149342542 | 6021 | 1: 149348963 | 6022 | 1: 149351545 | 6023 | 1: 149358100 |
| 6024 | 1: 149360084 | 6025 | 1: 149362014 | 6026 | 1: 149366388 | 6027 | 1: 149367120 | 6028 | 1: 149368535 |
| 6029 | 1: 149371783 | 6030 | 1: 149374604 | 6031 | 1: 149375645 | 6032 | 1: 149376574 | 6033 | 1: 149378550 |
| 6034 | 1: 149379934 | 6035 | 1: 149380878 | 6036 | 1: 149381954 | 6037 | 1: 149390100 | 6042 | 1: 149443669 |
| 6049 | 1: 149642436 | 6065 | 1: 149898235 | 6066 | 1: 149902664 | 6068 | 1: 149929034 | 6077 | 1: 150129027 |
| 6078 | 1: 150129035 | 6079 | 1: 150129044 | 6080 | 1: 150129048 | 6081 | 1: 150144069 | 6083 | 1: 150151876 |
| 6093 | 1: 150440522 | 6106 | 1: 150701995 | 6109 | 1: 150741379 | 6119 | 1: 151001062 | 6122 | 1: 151048846 |
| 6124 | 1: 151109660 | 6134 | 1: 151324257 | 6135 | 1: 151326467 | 6148 | 1: 151556745 | 6149 | 1: 151560943 |
| 6150 | 1: 151561380 | 6154 | 1: 151623827 | 6158 | 1: 151643959 | 6161 | 1: 151668710 | 6162 | 1: 151671521 |
| 6163 | 1: 151673956 | 6164 | 1: 151675356 | 6165 | 1: 151677189 | 6182 | 1: 151922660 | 6186 | 1: 152015544 |
| 6189 | 1: 152071824 | 6215 | 1: 152347862 | 6235 | 1: 152417708 | 6237 | 1: 152422584 | 6238 | 1: 152428562 |
| 6249 | 1: 152632298 | 6252 | 1: 152678335 | 6254 | 1: 152745164 | 6260 | 1: 152826141 | 6279 | 1: 153250410 |
| 6301 | 1: 153688732 | 6308 | 1: 153828051 | 6322 | 1: 154004623 | 6323 | 1: 154009064 | 6324 | 1: 154009137 |
| 6325 | 1: 154009145 | 6327 | 1: 154019636 | 6329 | 1: 154038751 | 6341 | 1: 154370113 | 6358 | 1: 154858681 |
| 6388 | 1: 155496781 | 6400 | 1: 155679617 | 6401 | 1: 155679654 | 6402 | 1: 155679691 | 6403 | 1: 155679758 |
| 6404 | 1: 155679762 | 6407 | 1: 155716904 | 6429 | 1: 156040352 | 6434 | 1: 156108198 | 6469 | 1: 156749778 |
| 6502 | 1: 157422591 | 6505 | 1: 157455223 | 6508 | 1: 157589021 | 6514 | 1: 157988428 | 6517 | 1: 158029386 |
| 6518 | 1: 158031759 | 6521 | 1: 158140847 | 6522 | 1: 158227949 | 6550 | 2: 773366 | 6570 | 2: 1116337 |
| 6571 | 2: 1120855 | 6576 | 2: 1174595 | 6579 | 2: 1234786 | 6580 | 2: 1239635 | 6584 | 2: 1280967 |
| 6585 | 2: 1281046 | 6586 | 2: 1286624 | 6587 | 2: 1286642 | 6588 | 2: 1286747 | 6607 | 2: 1669475 |
| 6615 | 2: 1857324 | 6616 | 2: 1859648 | 6623 | 2: 1996597 | 6642 | 2: 2733310 | 6649 | 2: 2936048 |
| 6657 | 2: 3205071 | 6671 | 2: 3644419 | 6679 | 2: 3866542 | 6680 | 2: 3866584 | 6688 | 2: 4033904 |
| 6692 | 2: 4056030 | 6693 | 2: 4056116 | 6702 | 2: 4244343 | 6705 | 2: 4311599 | 6706 | 2: 4311602 |
| 6707 | 2: 4311756 | 6708 | 2: 4318724 | 6710 | 2: 4360783 | 6719 | 2: 4631707 | 6720 | 2: 4634839 |
| 6721 | 2: 4634862 | 6722 | 2: 4634886 | 6723 | 2: 4634902 | 6724 | 2: 4634989 | 6725 | 2: 4655242 |
| 6727 | 2: 4687767 | 6731 | 2: 4782801 | 6749 | 2: 5105483 | 6754 | 2: 5312133 | 6757 | 2: 5343162 |
| 6760 | 2: 5372449 | 6767 | 2: 5418497 | 6770 | 2: 5433956 | 6771 | 2: 5437701 | 6776 | 2: 5564286 |
| 6778 | 2: 5598208 | 6780 | 2: 5605765 | 6781 | 2: 5668859 | 6786 | 2: 5824441 | 6806 | 2: 6219961 |
| 6807 | 2: 6220338 | 6820 | 2: 6413902 | 6826 | 2: 6665981 | 6827 | 2: 6670529 | 6828 | 2: 6670740 |
| 6829 | 2: 6670761 | 6830 | 2: 6670813 | 6831 | 2: 6670846 | 6842 | 2: 6830098 | 6844 | 2: 6891780 |
| 6846 | 2: 6946227 | 6851 | 2: 7030426 | 6852 | 2: 7053225 | 6855 | 2: 7145809 | 6859 | 2: 7201484 |
| 6860 | 2: 7214741 | 6861 | 2: 7228554 | 6865 | 2: 7278045 | 6867 | 2: 7321689 | 6869 | 2: 7334093 |
| 6870 | 2: 7339046 | 6871 | 2: 7346647 | 6874 | 2: 7389988 | 6876 | 2: 7414723 | 6877 | 2: 7487693 |
| 6880 | 2: 7550253 | 6893 | 2: 7824299 | 6894 | 2: 7854802 | 6895 | 2: 7854894 | 6896 | 2: 7854898 |
| 6903 | 2: 8149483 | 6907 | 2: 8268124 | 6909 | 2: 8273877 | 6925 | 2: 8874962 | 6926 | 2: 8891833 |
| 6948 | 2: 9583773 | 6949 | 2: 9583875 | 6957 | 2: 9791102 | 6960 | 2: 9831264 | 6985 | 2: 10595947 |
| 6986 | 2: 10596946 | 6987 | 2: 10597759 | 6988 | 2: 10603551 | 7032 | 2: 12051677 | 7037 | 2: 12305668 |
| 7043 | 2: 12489693 | 7058 | 2: 13087374 | 7071 | 2: 13360171 | 7072 | 2: 13382039 | 7082 | 2: 13695930 |
| 7084 | 2: 13709033 | 7086 | 2: 13779018 | 7088 | 2: 13797995 | 7090 | 2: 13822009 | 7091 | 2: 13823524 |
| 7092 | 2: 13862800 | 7102 | 2: 14216227 | 7127 | 2: 14784977 | 7139 | 2: 15119742 | 7147 | 2: 15329884 |
| 7157 | 2: 15549082 | 7162 | 2: 15690371 | 7163 | 2: 15693009 | 7164 | 2: 15696748 | 7168 | 2: 15821266 |
| 7173 | 2: 15967374 | 7190 | 2: 16339174 | 7207 | 2: 16790984 | 7211 | 2: 16932422 | 7217 | 2: 17092308 |
| 7223 | 2: 17311769 | 7236 | 2: 17583628 | 7241 | 2: 17659987 | 7242 | 2: 17735524 | 7246 | 2: 17833404 |
| 7250 | 2: 17967333 | 7254 | 2: 18038068 | 7260 | 2: 18147142 | 7261 | 2: 18165858 | 7263 | 2: 18229834 |
| 7264 | 2: 18229859 | 7266 | 2: 18241132 | 7268 | 2: 18244789 | 7270 | 2: 18253097 | 7271 | 2: 18254488 |
| 7273 | 2: 18262337 | 7275 | 2: 18270755 | 7276 | 2: 18271356 | 7277 | 2: 18271358 | 7278 | 2: 18271418 |
| 7279 | 2: 18272565 | 7280 | 2: 18272965 | 7281 | 2: 18275874 | 7282 | 2: 18277584 | 7283 | 2: 18277939 |
| 7284 | 2: 18280757 | 7285 | 2: 18280781 | 7286 | 2: 18282254 | 7287 | 2: 18282552 | 7288 | 2: 18290509 |
| 7289 | 2: 18291940 | 7290 | 2: 18307699 | 7291 | 2: 18323628 | 7293 | 2: 18378637 | 7297 | 2: 18502293 |
| 7304 | 2: 18586886 | 7305 | 2: 18595133 | 7306 | 2: 18605578 | 7310 | 2: 18642606 | 7314 | 2: 18778867 |
| 7316 | 2: 18866785 | 7317 | 2: 18869734 | 7325 | 2: 18981323 | 7326 | 2: 18986957 | 7330 | 2: 19045985 |
| 7335 | 2: 19136987 | 7346 | 2: 19340327 | 7352 | 2: 19534377 | 7355 | 2: 19593435 | 7356 | 2: 19626883 |
| 7357 | 2: 19648311 | 7358 | 2: 19648602 | 7368 | 2: 19897160 | 7369 | 2: 19918403 | 7378 | 2: 20155806 |
| 7380 | 2: 20264485 | 7383 | 2: 20344434 | 7387 | 2: 20427608 | 7394 | 2: 20674260 | 7401 | 2: 20845761 |
| 7403 | 2: 20861585 | 7407 | 2: 21064906 | 7421 | 2: 21401355 | 7422 | 2: 21405561 | 7428 | 2: 21553608 |
| 7438 | 2: 21688312 | 7443 | 2: 21849771 | 7444 | 2: 21867557 | 7445 | 2: 21867592 | 7447 | 2: 21914587 |
| 7449 | 2: 21967376 | 7451 | 2: 22019700 | 7452 | 2: 22051754 | 7458 | 2: 22154085 | 7466 | 2: 22396788 |
| 7472 | 2: 22599197 | 7474 | 2: 22608474 | 7479 | 2: 22767655 | 7480 | 2: 22787231 | 7485 | 2: 23032717 |
| 7489 | 2: 23063150 | 7496 | 2: 23295824 | 7497 | 2: 23312807 | 7502 | 2: 23459486 | 7504 | 2: 23476574 |
| 7505 | 2: 23481265 | 7506 | 2: 23481554 | 7507 | 2: 23481572 | 7509 | 2: 23487782 | 7510 | 2: 23501169 |
| 7511 | 2: 23537225 | 7512 | 2: 23557793 | 7513 | 2: 23558570 | 7520 | 2: 23643567 | 7522 | 2: 23666640 |
| 7523 | 2: 23676088 | 7525 | 2: 23691155 | 7526 | 2: 23696931 | 7527 | 2: 23699667 | 7534 | 2: 23962288 |
| 7536 | 2: 23971606 | 7545 | 2: 24172853 | 7546 | 2: 24176608 | 7552 | 2: 24299477 | 7554 | 2: 24320827 |
| 7555 | 2: 24320845 | 7575 | 2: 24669527 | 7586 | 2: 24895083 | 7591 | 2: 25095591 | 7595 | 2: 25243017 |
| 7597 | 2: 25329638 | 7600 | 2: 25433245 | 7601 | 2: 25441115 | 7602 | 2: 25443814 | 7606 | 2: 25523128 |
| 7607 | 2: 25531340 | 7608 | 2: 25539975 | 7609 | 2: 25556600 | 7617 | 2: 25649108 | 7620 | 2: 25710237 |
| 7658 | 2: 26534630 | 7667 | 2: 26760780 | 7669 | 2: 26762215 | 7670 | 2: 26771611 | 7671 | 2: 26774438 |
| 7674 | 2: 26851479 | 7691 | 2: 27096366 | 7698 | 2: 27312219 | 7702 | 2: 27432834 | 7727 | 2: 27915981 |
| 7736 | 2: 28189986 | 7738 | 2: 28287714 | 7739 | 2: 28287763 | 7740 | 2: 28287812 | 7741 | 2: 28287836 |
| 7746 | 2: 28373427 | 7749 | 2: 28418211 | 7764 | 2: 28631973 | 7780 | 2: 28937935 | 7813 | 2: 29825101 |
| 7817 | 2: 29924141 | 7825 | 2: 30218788 | 7826 | 2: 30225126 | 7827 | 2: 30235162 | 7828 | 2: 30235271 |
| 7837 | 2: 30489470 | 7840 | 2: 30505625 | 7847 | 2: 30620848 | 7850 | 2: 30714513 | 7852 | 2: 30729817 |
| 7867 | 2: 31118343 | 7868 | 2: 31118369 | 7869 | 2: 31121656 | 7887 | 2: 31621372 | 7905 | 2: 32163209 |
| 7921 | 2: 32683358 | 7923 | 2: 32754895 | 7930 | 2: 33004780 | 7937 | 2: 33199953 | 7948 | 2: 33575101 |
| 7949 | 2: 33575122 | 7950 | 2: 33654382 | 7952 | 2: 33671969 | 7953 | 2: 33671986 | 7954 | 2: 33694528 |
| 7959 | 2: 33883065 | 7962 | 2: 33960798 | 7978 | 2: 34316478 | 7981 | 2: 34353723 | 7986 | 2: 34439099 |
| 7989 | 2: 34507044 | 8027 | 2: 35881554 | 8035 | 2: 36123841 | 8036 | 2: 36138834 | 8037 | 2: 36147200 |
| 8051 | 2: 36510607 | 8055 | 2: 36554095 | 8056 | 2: 36613946 | 8057 | 2: 36621379 | 8060 | 2: 36742166 |
| 8075 | 2: 37300847 | 8101 | 2: 38053061 | 8107 | 2: 38237985 | 8108 | 2: 38238121 | 8109 | 2: 38238372 |
| 8110 | 2: 38238415 | 8111 | 2: 38238441 | 8112 | 2: 38238520 | 8113 | 2: 38238541 | 8117 | 2: 38310384 |
| 8121 | 2: 38442344 | 8123 | 2: 38465755 | 8130 | 2: 38600295 | 8131 | 2: 38634299 | 8136 | 2: 38811572 |
| 8163 | 2: 39460697 | 8203 | 2: 40835843 | 8205 | 2: 40867544 | 8206 | 2: 40867584 | 8207 | 2: 40867585 |
| 8208 | 2: 40867624 | 8209 | 2: 40867647 | 8210 | 2: 40867669 | 8211 | 2: 40867710 | 8212 | 2: 40867713 |
| 8215 | 2: 41050502 | 8216 | 2: 41050562 | 8217 | 2: 41050564 | 8218 | 2: 41050613 | 8226 | 2: 41315483 |
| 8227 | 2: 41315504 | 8245 | 2: 41559277 | 8253 | 2: 41775609 | 8254 | 2: 41797511 | 8287 | 2: 42520826 |
| 8288 | 2: 42535114 | 8307 | 2: 43006124 | 8321 | 2: 43491727 | 8323 | 2: 43580317 | 8324 | 2: 43631101 |
| 8328 | 2: 43711459 | 8332 | 2: 43813071 | 8341 | 2: 44152000 | 8343 | 2: 44193529 | 8350 | 2: 44379204 |
| 8351 | 2: 44393093 | 8352 | 2: 44393964 | 8353 | 2: 44403432 | 8356 | 2: 44515259 | 8357 | 2: 44545955 |
| 8358 | 2: 44569313 | 8360 | 2: 44615292 | 8361 | 2: 44621241 | 8367 | 2: 44858072 | 8384 | 2: 45372211 |
| 8388 | 2: 45474859 | 8412 | 2: 45983105 | 8415 | 2: 46033456 | 8420 | 2: 46147458 | 8428 | 2: 46391874 |
| 8434 | 2: 46437983 | 8436 | 2: 46446081 | 8440 | 2: 46534111 | 8446 | 2: 46705643 | 8464 | 2: 47013353 |
| 8467 | 2: 47100164 | 8481 | 2: 47433064 | 8486 | 2: 47596275 | 8491 | 2: 47706783 | 8509 | 2: 48215370 |
| 8522 | 2: 48603698 | 8523 | 2: 48604345 | 8526 | 2: 48755071 | 8528 | 2: 48783617 | 8531 | 2: 48830819 |
| 8534 | 2: 48894232 | 8539 | 2: 48987003 | 8544 | 2: 49067927 | 8555 | 2: 49577101 | 8571 | 2: 50088797 |
| 8590 | 2: 50645993 | 8595 | 2: 50810980 | 8605 | 2: 51033258 | 8615 | 2: 51338525 | 8626 | 2: 51770242 |
| 8628 | 2: 51813793 | 8649 | 2: 52303945 | 8655 | 2: 52440698 | 8660 | 2: 52515449 | 8671 | 2: 52834508 |
| 8675 | 2: 52903006 | 8676 | 2: 52906421 | 8677 | 2: 52909824 | 8681 | 2: 52959066 | 8690 | 2: 53226738 |
| 8735 | 2: 54491736 | 8753 | 2: 54901841 | 8756 | 2: 54993038 | 8757 | 2: 54993081 | 8758 | 2: 54993084 |
| 8759 | 2: 54993089 | 8761 | 2: 55019715 | 8769 | 2: 55150342 | 8770 | 2: 55235366 | 8800 | 2: 56234809 |
| 8802 | 2: 56280951 | 8804 | 2: 56314003 | 8809 | 2: 56469340 | 8816 | 2: 56646269 | 8838 | 2: 57404778 |
| 8839 | 2: 57430549 | 8849 | 2: 57666386 | 8851 | 2: 57700250 | 8858 | 2: 57837920 | 8888 | 2: 58761910 |
| 8890 | 2: 58778117 | 8892 | 2: 58793131 | 8897 | 2: 58929038 | 8899 | 2: 58940947 | 8901 | 2: 58993535 |
| 8903 | 2: 59042027 | 8904 | 2: 59079693 | 8908 | 2: 59202049 | 8909 | 2: 59212187 | 8923 | 2: 59601537 |
| 8931 | 2: 59858246 | 8932 | 2: 59858284 | 8933 | 2: 59858319 | 8934 | 2: 59858339 | 8949 | 2: 60150792 |
| 8967 | 2: 60751699 | 8978 | 2: 61108555 | 8980 | 2: 61144456 | 8981 | 2: 61170888 | 8982 | 2: 61172063 |
| 8985 | 2: 61252370 | 8992 | 2: 61343961 | 8994 | 2: 61449061 | 8995 | 2: 61490160 | 8996 | 2: 61490261 |
| 8998 | 2: 61494582 | 9000 | 2: 61535058 | 9001 | 2: 61535147 | 9002 | 2: 61535149 | 9003 | 2: 61535213 |
| 9013 | 2: 61843883 | 9020 | 2: 62046115 | 9024 | 2: 62302155 | 9046 | 2: 62786824 | 9051 | 2: 62910878 |
| 9052 | 2: 62944872 | 9053 | 2: 62944925 | 9055 | 2: 63002162 | 9065 | 2: 63241642 | 9070 | 2: 63325322 |
| 9078 | 2: 63563631 | 9081 | 2: 63616590 | 9090 | 2: 63926681 | 9124 | 2: 64713810 | 9129 | 2: 64820071 |
| 9148 | 2: 65218951 | 9164 | 2: 65726104 | 9221 | 2: 67528810 | 9226 | 2: 67669505 | 9236 | 2: 68034573 |
| 9267 | 2: 69100324 | 9283 | 2: 69720593 | 9294 | 2: 70008307 | 9303 | 2: 70294251 | 9304 | 2: 70313073 |
| 9310 | 2: 70440178 | 9324 | 2: 70768201 | 9329 | 2: 70931242 | 9334 | 2: 71041850 | 9335 | 2: 71042132 |
| 9336 | 2: 71042190 | 9337 | 2: 71044225 | 9338 | 2: 71095900 | 9341 | 2: 71203819 | 9342 | 2: 71205559 |
| 9349 | 2: 71337903 | 9357 | 2: 71501065 | 9366 | 2: 71912340 | 9375 | 2: 72314326 | 9392 | 2: 72649795 |
| 9393 | 2: 72669915 | 9394 | 2: 72669943 | 9403 | 2: 72918814 | 9404 | 2: 72977278 | 9405 | 2: 72995902 |
| 9407 | 2: 73090036 | 9413 | 2: 73157226 | 9414 | 2: 73160964 | 9415 | 2: 73161983 | 9420 | 2: 73383708 |
| 9422 | 2: 73398069 | 9424 | 2: 73477830 | 9434 | 2: 73757540 | 9437 | 2: 73811343 | 9446 | 2: 73945664 |
| 9449 | 2: 74083815 | 9468 | 2: 74707536 | 9469 | 2: 74707559 | 9470 | 2: 74707612 | 9471 | 2: 74707658 |
| 9472 | 2: 74707710 | 9482 | 2: 75029605 | 9485 | 2: 75173951 | 9493 | 2: 75482167 | 9494 | 2: 75483513 |
| 9501 | 2: 75692826 | 9507 | 2: 75820235 | 9508 | 2: 75834257 | 9517 | 2: 76002206 | 9519 | 2: 76048349 |
| 9527 | 2: 76266636 | 9537 | 2: 76475875 | 9539 | 2: 76503911 | 9556 | 2: 77018992 | 9561 | 2: 77216172 |
| 9563 | 2: 77216223 | 9570 | 2: 77393386 | 9571 | 2: 77423386 | 9575 | 2: 77530544 | 9579 | 2: 77676860 |
| 9580 | 2: 77680269 | 9581 | 2: 77690134 | 9583 | 2: 77796311 | 9590 | 2: 77930065 | 9591 | 2: 77960137 |
| 9592 | 2: 77960148 | 9593 | 2: 77960205 | 9594 | 2: 77960221 | 9595 | 2: 78058933 | 9599 | 2: 78093605 |
| 9608 | 2: 78475004 | 9610 | 2: 78576254 | 9621 | 2: 78787744 | 9622 | 2: 78787785 | 9623 | 2: 78793670 |
| 9625 | 2: 78828241 | 9627 | 2: 78856439 | 9631 | 2: 78919311 | 9632 | 2: 78922277 | 9634 | 2: 78933501 |
| 9635 | 2: 78933506 | 9636 | 2: 78940906 | 9637 | 2: 78942899 | 9640 | 2: 79034805 | 9643 | 2: 79043516 |
| 9665 | 2: 79805247 | 9673 | 2: 79959399 | 9683 | 2: 80095415 | 9686 | 2: 80170719 | 9762 | 2: 83630529 |
| 9763 | 2: 83648977 | 9764 | 2: 83654198 | 9765 | 2: 83657997 | 9766 | 2: 83660404 | 9767 | 2: 83664117 |
| 9768 | 2: 83664826 | 9769 | 2: 83665834 | 9770 | 2: 83667393 | 9771 | 2: 83669899 | 9772 | 2: 83671239 |
| 9774 | 2: 83694268 | 9775 | 2: 83694872 | 9776 | 2: 83696501 | 9777 | 2: 83697095 | 9778 | 2: 83701382 |
| 9779 | 2: 83703221 | 9780 | 2: 83710452 | 9796 | 2: 84314672 | 9801 | 2: 84413080 | 9806 | 2: 84544851 |
| 9807 | 2: 84554472 | 9808 | 2: 84556946 | 9809 | 2: 84558765 | 9810 | 2: 84558813 | 9811 | 2: 84579866 |
| 9812 | 2: 84579884 | 9814 | 2: 84594590 | 9815 | 2: 84594621 | 9816 | 2: 84631977 | 9821 | 2: 84747533 |
| 9826 | 2: 84895919 | 9829 | 2: 84986688 | 9841 | 2: 85268807 | 9842 | 2: 85302489 | 9857 | 2: 85748841 |
| 9868 | 2: 85921857 | 9870 | 2: 85984791 | 9871 | 2: 86008072 | 9876 | 2: 86150518 | 9881 | 2: 86310570 |
| 9882 | 2: 86387502 | 9886 | 2: 86419348 | 9889 | 2: 86436688 | 9891 | 2: 86469285 | 9894 | 2: 86509868 |
| 9961 | 2: 88510673 | 9965 | 2: 88623312 | 9970 | 2: 88707441 | 9971 | 2: 88719162 | 9973 | 2: 88746032 |
| 9975 | 2: 88782810 | 9977 | 2: 88859157 | 9983 | 2: 89070767 | 9984 | 2: 89076240 | 9985 | 2: 89084301 |
| 9986 | 2: 89086388 | 9987 | 2: 89095014 | 9988 | 2: 89100826 | 9989 | 2: 89104328 | 9990 | 2: 89109055 |
| 9991 | 2: 89114149 | 9992 | 2: 89117889 | 9993 | 2: 89119778 | 9994 | 2: 89122696 | 9995 | 2: 89126698 |
| 9996 | 2: 89128778 | 9997 | 2: 89129756 | 9998 | 2: 89130672 | 9999 | 2: 89131642 | 10000 | 2: 89132131 |
| 10001 | 2: 89132635 | 10002 | 2: 89133746 | 10003 | 2: 89136358 | 10004 | 2: 89136948 | 10005 | 2: 89143985 |
| 10006 | 2: 89150132 | 10007 | 2: 89157341 | 10008 | 2: 89161844 | 10009 | 2: 89164769 | 10010 | 2: 89172177 |
| 10011 | 2: 89183562 | 10012 | 2: 89188097 | 10013 | 2: 89192815 | 10014 | 2: 89196778 | 10015 | 2: 89206007 |
| 10016 | 2: 89210274 | 10017 | 2: 89220562 | 10021 | 2: 89293059 | 10022 | 2: 89329459 | 10023 | 2: 89330715 |
| 10024 | 2: 89332510 | 10025 | 2: 89333883 | 10026 | 2: 89335072 | 10028 | 2: 89337942 | 10031 | 2: 89377233 |
| 10034 | 2: 89445178 | 10049 | 2: 89808466 | 10050 | 2: 89813015 | 10052 | 2: 89841096 | 10054 | 2: 89876885 |
| 10055 | 2: 89878690 | 10057 | 2: 89908253 | 10059 | 2: 89916678 | 10060 | 2: 89948655 | 10061 | 2: 89952700 |
| 10062 | 2: 89952808 | 10063 | 2: 89953249 | 10064 | 2: 89964009 | 10065 | 2: 89964459 | 10066 | 2: 89964908 |
| 10100 | 2: 91309327 | 10101 | 2: 91319564 | 10123 | 2: 92012717 | 10131 | 2: 92095096 | 10132 | 2: 92109122 |
| 10142 | 2: 92406522 | 10143 | 2: 92416012 | 10146 | 2: 92501151 | 10148 | 2: 92504237 | 10154 | 2: 92593249 |
| 10157 | 2: 92677123 | 10158 | 2: 92688749 | 10163 | 2: 92792925 | 10165 | 2: 92800370 | 10167 | 2: 92859552 |
| 10168 | 2: 92875951 | 10171 | 2: 93007619 | 10197 | 2: 93432778 | 10217 | 2: 93888418 | 10231 | 2: 94155809 |
| 10233 | 2: 94177314 | 10234 | 2: 94238285 | 10238 | 2: 94284858 | 10242 | 2: 94408041 | 10244 | 2: 94532812 |
| 10250 | 2: 94662652 | 10263 | 2: 94943212 | 10269 | 2: 95104701 | 10273 | 2: 95191751 | 10276 | 2: 95262942 |
| 10277 | 2: 95265410 | 10297 | 2: 95781931 | 10298 | 2: 95788314 | 10299 | 2: 95797570 | 10304 | 2: 95957388 |
| 10307 | 2: 96027434 | 10312 | 2: 96139223 | 10313 | 2: 96162249 | 10321 | 2: 96318089 | 10324 | 2: 96352046 |
| 10330 | 2: 96463623 | 10332 | 2: 96505449 | 10333 | 2: 96512869 | 10334 | 2: 96514006 | 10335 | 2: 96514035 |
| 10336 | 2: 96514066 | 10337 | 2: 96514071 | 10339 | 2: 96516949 | 10340 | 2: 96517000 | 10341 | 2: 96518404 |
| 10342 | 2: 96519726 | 10343 | 2: 96519764 | 10344 | 2: 96519823 | 10345 | 2: 96522707 | 10346 | 2: 96522837 |
| 10352 | 2: 96721652 | 10373 | 2: 97569993 | 10393 | 2: 98098074 | 10394 | 2: 98098574 | 10397 | 2: 98189476 |
| 10411 | 2: 98434332 | 10435 | 2: 99027387 | 10443 | 2: 99309195 | 10444 | 2: 99310709 | 10477 | 2: 100060201 |
| 10486 | 2: 100359058 | 10488 | 2: 100436598 | 10489 | 2: 100436640 | 10490 | 2: 100507665 | 10491 | 2: 100527030 |
| 10508 | 2: 100969194 | 10512 | 2: 101142510 | 10515 | 2: 101176628 | 10516 | 2: 101267741 | 10522 | 2: 101470754 |
| 10524 | 2: 101503694 | 10526 | 2: 101544902 | 10529 | 2: 101696463 | 10540 | 2: 102030097 | 10541 | 2: 102031180 |
| 10567 | 2: 102763970 | 10569 | 2: 102854361 | 10570 | 2: 102861535 | 10574 | 2: 103021453 | 10575 | 2: 103022409 |
| 10577 | 2: 103050047 | 10578 | 2: 103087349 | 10588 | 2: 103396204 | 10589 | 2: 103402752 | 10592 | 2: 103445587 |
| 10595 | 2: 103517827 | 10598 | 2: 103593199 | 10610 | 2: 103884686 | 10615 | 2: 104008673 | 10622 | 2: 104115856 |
| 10628 | 2: 104308034 | 10635 | 2: 104472870 | 10641 | 2: 104678498 | 10643 | 2: 104790859 | 10644 | 2: 104814826 |
| 10646 | 2: 104850637 | 10647 | 2: 104882698 | 10660 | 2: 105042627 | 10669 | 2: 105160301 | 10679 | 2: 105493554 |
| 10682 | 2: 105532073 | 10684 | 2: 105558282 | 10685 | 2: 105589302 | 10689 | 2: 105726409 | 10697 | 2: 105850938 |
| 10698 | 2: 105883087 | 10702 | 2: 105943923 | 10704 | 2: 106005198 | 10705 | 2: 106040867 | 10708 | 2: 106093074 |
| 10709 | 2: 106134401 | 10716 | 2: 106279517 | 10718 | 2: 106396433 | 10719 | 2: 106446238 | 10720 | 2: 106455619 |
| 10722 | 2: 106582615 | 10724 | 2: 106599852 | 10725 | 2: 106602909 | 10726 | 2: 106605195 | 10727 | 2: 106609300 |
| 10728 | 2: 106609491 | 10729 | 2: 106615459 | 10730 | 2: 106624131 | 10731 | 2: 106632743 | 10732 | 2: 106635677 |
| 10740 | 2: 106788337 | 10748 | 2: 106865812 | 10750 | 2: 106960370 | 10752 | 2: 107059040 | 10753 | 2: 107076804 |
| 10766 | 2: 107558581 | 10768 | 2: 107560640 | 10769 | 2: 107560655 | 10773 | 2: 107620491 | 10774 | 2: 107628799 |
| 10777 | 2: 107692575 | 10780 | 2: 107718206 | 10793 | 2: 107772997 | 10796 | 2: 107824497 | 10797 | 2: 107827321 |
| 10799 | 2: 107881110 | 10808 | 2: 108144100 | 10810 | 2: 108165798 | 10820 | 2: 108449215 | 10827 | 2: 108649909 |
| 10835 | 2: 108855822 | 10838 | 2: 108918709 | 10839 | 2: 108932264 | 10841 | 2: 108944061 | 10850 | 2: 109135684 |
| 10861 | 2: 109403406 | 10862 | 2: 109415989 | 10867 | 2: 109507997 | 10868 | 2: 109525518 | 10871 | 2: 109605505 |
| 10874 | 2: 109627420 | 10878 | 2: 109678083 | 10896 | 2: 110207989 | 10909 | 2: 110556883 | 10919 | 2: 111069391 |
| 10923 | 2: 111211200 | 10924 | 2: 111216842 | 10931 | 2: 111473451 | 10946 | 2: 111903128 | 10950 | 2: 112002693 |
| 10951 | 2: 112064672 | 10952 | 2: 112068968 | 10963 | 2: 112309161 | 10982 | 2: 112824303 | 10985 | 2: 112865726 |
| 10988 | 2: 112884446 | 10996 | 2: 112999914 | 10998 | 2: 113039809 | 11000 | 2: 113071684 | 11004 | 2: 113217166 |
| 11005 | 2: 113217179 | 11006 | 2: 113217208 | 11007 | 2: 113217254 | 11033 | 2: 113775291 | 11040 | 2: 113927720 |
| 11052 | 2: 114312086 | 11056 | 2: 114441340 | 11057 | 2: 114511997 | 11070 | 2: 114795811 | 11072 | 2: 114857637 |
| 11087 | 2: 115277541 | 11088 | 2: 115279258 | 11092 | 2: 115316715 | 11096 | 2: 115405473 | 11097 | 2: 115455721 |
| 11106 | 2: 115621193 | 11108 | 2: 115625826 | 11125 | 2: 115953383 | 11155 | 2: 116733465 | 11161 | 2: 116888348 |
| 11167 | 2: 117033411 | 11168 | 2: 117047832 | 11169 | 2: 117079799 | 11172 | 2: 117133057 | 11178 | 2: 117240992 |
| 11187 | 2: 117466579 | 11195 | 2: 117945652 | 11200 | 2: 118099830 | 11207 | 2: 118201421 | 11220 | 2: 118509097 |
| 11222 | 2: 118554241 | 11239 | 2: 119145772 | 11240 | 2: 119196348 | 11241 | 2: 119198191 | 11242 | 2: 119199613 |
| 11244 | 2: 119244130 | 11245 | 2: 119264906 | 11248 | 2: 119291176 | 11252 | 2: 119322052 | 11257 | 2: 119465613 |
| 11260 | 2: 119511934 | 11261 | 2: 119512418 | 11262 | 2: 119522741 | 11263 | 2: 119526528 | 11264 | 2: 119533194 |
| 11267 | 2: 119548536 | 11269 | 2: 119562713 | 11270 | 2: 119564586 | 11271 | 2: 119565180 | 11272 | 2: 119566000 |
| 11273 | 2: 119576736 | 11277 | 2: 119825482 | 11278 | 2: 119862058 | 11290 | 2: 120178985 | 11292 | 2: 120195958 |
| 11293 | 2: 120199797 | 11308 | 2: 120568141 | 11310 | 2: 120671914 | 11319 | 2: 120900260 | 11322 | 2: 120932791 |
| 11331 | 2: 121151913 | 11333 | 2: 121188721 | 11352 | 2: 121755672 | 11358 | 2: 121893423 | 11359 | 2: 121894763 |
| 11375 | 2: 122580635 | 11382 | 2: 122800513 | 11385 | 2: 122878392 | 11393 | 2: 123070777 | 11394 | 2: 123070905 |
| 11395 | 2: 123070944 | 11396 | 2: 123100345 | 11397 | 2: 123100370 | 11414 | 2: 123539540 | 11415 | 2: 123539541 |
| 11417 | 2: 123562349 | 11419 | 2: 123590694 | 11420 | 2: 123590830 | 11421 | 2: 123610591 | 11427 | 2: 123692694 |
| 11447 | 2: 124332744 | 11453 | 2: 124600152 | 11458 | 2: 124710998 | 11462 | 2: 124913543 | 11470 | 2: 125166728 |
| 11476 | 2: 125250396 | 11479 | 2: 125277549 | 11480 | 2: 125286027 | 11481 | 2: 125349483 | 11490 | 2: 125625209 |
| 11492 | 2: 125653640 | 11493 | 2: 125653952 | 11496 | 2: 125742333 | 11499 | 2: 125810128 | 11502 | 2: 125931055 |
| 11503 | 2: 125952736 | 11508 | 2: 126114829 | 11511 | 2: 126173762 | 11515 | 2: 126267198 | 11516 | 2: 126267881 |
| 11517 | 2: 126273576 | 11518 | 2: 126275683 | 11519 | 2: 126286517 | 11522 | 2: 126366193 | 11523 | 2: 126367550 |
| 11525 | 2: 126372344 | 11526 | 2: 126380236 | 11529 | 2: 126489135 | 11575 | 2: 127003970 | 11576 | 2: 127004702 |
| 11583 | 2: 127018411 | 11587 | 2: 127027686 | 11589 | 2: 127038523 | 11606 | 2: 127110467 | 11613 | 2: 127238953 |
| 11623 | 2: 127397394 | 11629 | 2: 127525976 | 11634 | 2: 127633593 | 11636 | 2: 127635918 | 11637 | 2: 127639135 |
| 11654 | 2: 128049090 | 11656 | 2: 128099391 | 11672 | 2: 128508443 | 11673 | 2: 128508479 | 11677 | 2: 128603208 |
| 11683 | 2: 128718168 | 11688 | 2: 128782288 | 11691 | 2: 128791601 | 11692 | 2: 128804408 | 11695 | 2: 128916878 |
| 11701 | 2: 129068395 | 11702 | 2: 129073892 | 11704 | 2: 129084385 | 11705 | 2: 129095315 | 11706 | 2: 129098350 |
| 11707 | 2: 129101237 | 11719 | 2: 129414787 | 11721 | 2: 129451897 | 11722 | 2: 129453738 | 11723 | 2: 129463584 |
| 11724 | 2: 129475673 | 11725 | 2: 129514375 | 11735 | 2: 129823411 | 11740 | 2: 129952968 | 11750 | 2: 130179342 |
| 11751 | 2: 130179458 | 11753 | 2: 130297720 | 11759 | 2: 130405723 | 11761 | 2: 130416105 | 11771 | 2: 130763239 |
| 11773 | 2: 130778311 | 11775 | 2: 130786713 | 11777 | 2: 130887344 | 11779 | 2: 130922023 | 11780 | 2: 130922555 |
| 11781 | 2: 130924042 | 11786 | 2: 131056297 | 11788 | 2: 131116831 | 11792 | 2: 131182393 | 11793 | 2: 131183245 |
| 11799 | 2: 131239243 | 11810 | 2: 131495314 | 11812 | 2: 131522458 | 11818 | 2: 131759255 | 11827 | 2: 131997425 |
| 11828 | 2: 132032080 | 11829 | 2: 132052956 | 11836 | 2: 132152838 | 11850 | 2: 132304236 | 11856 | 2: 132453956 |
| 11862 | 2: 132630596 | 11873 | 2: 132828927 | 11874 | 2: 132829391 | 11883 | 2: 133050393 | 11884 | 2: 133069759 |
| 11886 | 2: 133077482 | 11888 | 2: 133094549 | 11889 | 2: 133095512 | 11892 | 2: 133207446 | 11897 | 2: 133305481 |
| 11901 | 2: 133364775 | 11905 | 2: 133421233 | 11908 | 2: 133536795 | 11910 | 2: 133542411 | 11915 | 2: 133623325 |
| 11923 | 2: 133694191 | 11927 | 2: 133778125 | 11928 | 2: 133778616 | 11929 | 2: 133785516 | 11931 | 2: 133786535 |
| 11932 | 2: 133787224 | 11933 | 2: 133787956 | 11934 | 2: 133787982 | 11935 | 2: 133788783 | 11957 | 2: 134106505 |
| 11963 | 2: 134189401 | 11964 | 2: 134189458 | 11965 | 2: 134190122 | 11966 | 2: 134190219 | 11977 | 2: 134376164 |
| 11979 | 2: 134438884 | 11982 | 2: 134480181 | 11983 | 2: 134480249 | 11984 | 2: 134480295 | 11985 | 2: 134480304 |
| 11988 | 2: 134508261 | 11997 | 2: 134616684 | 11998 | 2: 134616699 | 12008 | 2: 134803486 | 12012 | 2: 134834209 |
| 12013 | 2: 134843772 | 12019 | 2: 134917533 | 12020 | 2: 134917563 | 12021 | 2: 134917597 | 12022 | 2: 134917643 |
| 12023 | 2: 134917658 | 12031 | 2: 135091858 | 12033 | 2: 135100100 | 12035 | 2: 135114924 | 12038 | 2: 135166182 |
| 12039 | 2: 135171128 | 12042 | 2: 135233030 | 12043 | 2: 135235158 | 12048 | 2: 135319271 | 12054 | 2: 135454017 |
| 12058 | 2: 135514495 | 12063 | 2: 135598504 | 12064 | 2: 135598610 | 12066 | 2: 135604349 | 12069 | 2: 135646413 |
| 12074 | 2: 135709576 | 12076 | 2: 135727874 | 12077 | 2: 135727928 | 12079 | 2: 135837161 | 12080 | 2: 135837263 |
| 12082 | 2: 135865945 | 12083 | 2: 135887710 | 12084 | 2: 135900620 | 12085 | 2: 135900833 | 12086 | 2: 135904261 |
| 12087 | 2: 135908217 | 12091 | 2: 136093517 | 12092 | 2: 136093612 | 12104 | 3: 197807 | 12127 | 3: 746327 |
| 12135 | 3: 867632 | 12139 | 3: 923989 | 12140 | 3: 924352 | 12142 | 3: 941811 | 12146 | 3: 969297 |
| 12155 | 3: 1114174 | 12162 | 3: 1183922 | 12181 | 3: 1651460 | 12184 | 3: 1672587 | 12188 | 3: 1729118 |
| 12195 | 3: 1939081 | 12198 | 3: 1965533 | 12199 | 3: 1967307 | 12204 | 3: 2023241 | 12206 | 3: 2062805 |
| 12208 | 3: 2064509 | 12211 | 3: 2089682 | 12212 | 3: 2098556 | 12213 | 3: 2104449 | 12235 | 3: 2740113 |
| 12243 | 3: 2986445 | 12245 | 3: 2993307 | 12250 | 3: 3052396 | 12283 | 3: 3863731 | 12305 | 3: 4250424 |
| 12306 | 3: 4264560 | 12307 | 3: 4280470 | 12308 | 3: 4285570 | 12310 | 3: 4298685 | 12312 | 3: 4313521 |
| 12314 | 3: 4363949 | 12316 | 3: 4398654 | 12332 | 3: 4566642 | 12335 | 3: 4593309 | 12338 | 3: 4692667 |
| 12344 | 3: 4732630 | 12345 | 3: 4732659 | 12354 | 3: 4874925 | 12355 | 3: 4874954 | 12373 | 3: 5268053 |
| 12377 | 3: 5309746 | 12383 | 3: 5436199 | 12384 | 3: 5462355 | 12408 | 3: 5998482 | 12422 | 3: 6207469 |
| 12424 | 3: 6282733 | 12425 | 3: 6293053 | 12432 | 3: 6517775 | 12443 | 3: 6741890 | 12444 | 3: 6771925 |
| 12445 | 3: 6771928 | 12446 | 3: 6771969 | 12447 | 3: 6772038 | 12469 | 3: 7060982 | 12470 | 3: 7106174 |
| 12478 | 3: 7235310 | 12479 | 3: 7238069 | 12481 | 3: 7255164 | 12483 | 3: 7357130 | 12486 | 3: 7383219 |
| 12492 | 3: 7455499 | 12506 | 3: 7653471 | 12511 | 3: 7783911 | 12512 | 3: 7822809 | 12514 | 3: 7838838 |
| 12522 | 3: 7931685 | 12523 | 3: 7931828 | 12524 | 3: 7931887 | 12528 | 3: 7961056 | 12537 | 3: 8147516 |
| 12538 | 3: 8163086 | 12539 | 3: 8174824 | 12540 | 3: 8183497 | 12541 | 3: 8184872 | 12542 | 3: 8186958 |
| 12543 | 3: 8193625 | 12544 | 3: 8194122 | 12545 | 3: 8208066 | 12551 | 3: 8277038 | 12552 | 3: 8300299 |
| 12555 | 3: 8337519 | 12570 | 3: 8661008 | 12584 | 3: 9427220 | 12587 | 3: 9485193 | 12588 | 3: 9485906 |
| 12590 | 3: 9513246 | 12594 | 3: 9564986 | 12595 | 3: 9591739 | 12598 | 3: 9636755 | 12605 | 3: 9764080 |
| 12606 | 3: 9832615 | 12608 | 3: 9847485 | 12609 | 3: 9858051 | 12612 | 3: 9863664 | 12622 | 3: 10118782 |
| 12623 | 3: 10118907 | 12624 | 3: 10119257 | 12625 | 3: 10119319 | 12626 | 3: 10122375 | 12627 | 3: 10122970 |
| 12628 | 3: 10123769 | 12629 | 3: 10128298 | 12630 | 3: 10132847 | 12631 | 3: 10133516 | 12632 | 3: 10134377 |
| 12633 | 3: 10136175 | 12634 | 3: 10137707 | 12635 | 3: 10140467 | 12636 | 3: 10141169 | 12637 | 3: 10156326 |
| 12638 | 3: 10159182 | 12639 | 3: 10177781 | 12641 | 3: 10178026 | 12642 | 3: 10178076 | 12643 | 3: 10178165 |
| 12644 | 3: 10178365 | 12645 | 3: 10178537 | 12652 | 3: 10343580 | 12656 | 3: 10373626 | 12662 | 3: 10458829 |
| 12663 | 3: 10458901 | 12664 | 3: 10458904 | 12665 | 3: 10458919 | 12666 | 3: 10458930 | 12667 | 3: 10458938 |
| 12668 | 3: 10459027 | 12669 | 3: 10459091 | 12670 | 3: 10459139 | 12671 | 3: 10459202 | 12672 | 3: 10459244 |
| 12673 | 3: 10459247 | 12674 | 3: 10459248 | 12675 | 3: 10459299 | 12676 | 3: 10459407 | 12677 | 3: 10459424 |
| 12679 | 3: 10497816 | 12680 | 3: 10497887 | 12681 | 3: 10497945 | 12682 | 3: 10497948 | 12683 | 3: 10497963 |
| 12684 | 3: 10498011 | 12686 | 3: 10519036 | 12688 | 3: 10519145 | 12689 | 3: 10526199 | 12690 | 3: 10526562 |
| 12691 | 3: 10526604 | 12692 | 3: 10527562 | 12693 | 3: 10527602 | 12694 | 3: 10527675 | 12700 | 3: 10725165 |
| 12703 | 3: 10854110 | 12704 | 3: 10854114 | 12705 | 3: 10854133 | 12706 | 3: 10854173 | 12707 | 3: 10854726 |
| 12708 | 3: 10854773 | 12710 | 3: 10868768 | 12738 | 3: 11492432 | 12739 | 3: 11524924 | 12740 | 3: 11525352 |
| 12741 | 3: 11525876 | 12744 | 3: 11607381 | 12746 | 3: 11634350 | 12747 | 3: 11634398 | 12748 | 3: 11635619 |
| 12762 | 3: 12154189 | 12786 | 3: 12876946 | 12792 | 3: 13018051 | 12799 | 3: 13164663 | 12815 | 3: 13606552 |
| 12818 | 3: 13623598 | 12819 | 3: 13623632 | 12820 | 3: 13624757 | 12829 | 3: 13851188 | 12839 | 3: 13973650 |
| 12841 | 3: 13997525 | 12844 | 3: 14032792 | 12848 | 3: 14146757 | 12849 | 3: 14158851 | 12850 | 3: 14172051 |
| 12851 | 3: 14184603 | 12852 | 3: 14185315 | 12853 | 3: 14185422 | 12854 | 3: 14188705 | 12857 | 3: 14233203 |
| 12858 | 3: 14239901 | 12859 | 3: 14259325 | 12862 | 3: 14312950 | 12867 | 3: 14498473 | 12868 | 3: 14500940 |
| 12870 | 3: 14546904 | 12874 | 3: 14640547 | 12877 | 3: 14754130 | 12879 | 3: 14774051 | 12883 | 3: 14797864 |
| 12888 | 3: 14853239 | 12891 | 3: 14983274 | 12895 | 3: 15110207 | 12897 | 3: 15181630 | 12899 | 3: 15254497 |
| 12904 | 3: 15408885 | 12905 | 3: 15410634 | 12906 | 3: 15410653 | 12907 | 3: 15410660 | 12911 | 3: 15464742 |
| 12915 | 3: 15536943 | 12917 | 3: 15587630 | 12921 | 3: 15632301 | 12923 | 3: 15659384 | 12929 | 3: 15702216 |
| 12932 | 3: 15793894 | 12933 | 3: 15794524 | 12945 | 3: 15926790 | 12950 | 3: 16002303 | 12955 | 3: 16101556 |
| 12956 | 3: 16101690 | 12957 | 3: 16153064 | 12961 | 3: 16228126 | 12964 | 3: 16347204 | 12978 | 3: 16700799 |
| 12979 | 3: 16719574 | 12980 | 3: 16719945 | 12981 | 3: 16720763 | 12982 | 3: 16720823 | 12983 | 3: 16720842 |
| 12984 | 3: 16721218 | 12985 | 3: 16723559 | 12986 | 3: 16725242 | 13003 | 3: 17249309 | 13035 | 3: 18290761 |
| 13058 | 3: 18945520 | 13061 | 3: 19021728 | 13063 | 3: 19054577 | 13070 | 3: 19211257 | 13071 | 3: 19217706 |
| 13072 | 3: 19217713 | 13073 | 3: 19218362 | 13074 | 3: 19225387 | 13075 | 3: 19225406 | 13076 | 3: 19225421 |
| 13077 | 3: 19225424 | 13078 | 3: 19225426 | 13079 | 3: 19225509 | 13082 | 3: 19277608 | 13089 | 3: 19513415 |
| 13094 | 3: 19685974 | 13097 | 3: 19711385 | 13099 | 3: 19775942 | 13101 | 3: 19816181 | 13109 | 3: 20143190 |
| 13110 | 3: 20145668 | 13112 | 3: 20160583 | 13116 | 3: 20338589 | 13123 | 3: 20549806 | 13125 | 3: 20564672 |
| 13130 | 3: 20805323 | 13132 | 3: 20877656 | 13142 | 3: 21424575 | 13168 | 3: 22009877 | 13173 | 3: 22059782 |
| 13178 | 3: 22108825 | 13201 | 3: 22592195 | 13206 | 3: 22780511 | 13207 | 3: 22808543 | 13209 | 3: 22841288 |
| 13211 | 3: 22869028 | 13214 | 3: 22938448 | 13215 | 3: 22963089 | 13219 | 3: 23051090 | 13220 | 3: 23087313 |
| 13225 | 3: 23186080 | 13231 | 3: 23299798 | 13233 | 3: 23367141 | 13235 | 3: 23401470 | 13242 | 3: 23543571 |
| 13259 | 3: 24023430 | 13260 | 3: 24023439 | 13265 | 3: 24145693 | 13266 | 3: 24151362 | 13269 | 3: 24196352 |
| 13270 | 3: 24213490 | 13272 | 3: 24238856 | 13281 | 3: 24394654 | 13283 | 3: 24424423 | 13301 | 3: 24950077 |
| 13308 | 3: 25150752 | 13310 | 3: 25164615 | 13311 | 3: 25170221 | 13312 | 3: 25173303 | 13331 | 3: 25597219 |
| 13332 | 3: 25600844 | 13344 | 3: 26002177 | 13346 | 3: 26027856 | 13347 | 3: 26036214 | 13348 | 3: 26044741 |
| 13349 | 3: 26046291 | 13359 | 3: 26212407 | 13360 | 3: 26212494 | 13361 | 3: 26212691 | 13363 | 3: 26256361 |
| 13371 | 3: 26380532 | 13372 | 3: 26391670 | 13377 | 3: 26439980 | 13378 | 3: 26469071 | 13379 | 3: 26473706 |
| 13391 | 3: 26893372 | 13395 | 3: 26962431 | 13397 | 3: 26979935 | 13399 | 3: 27052772 | 13400 | 3: 27057546 |
| 13401 | 3: 27081349 | 13409 | 3: 27180648 | 13413 | 3: 27264556 | 13414 | 3: 27294196 | 13417 | 3: 27380069 |
| 13418 | 3: 27382883 | 13419 | 3: 27384412 | 13423 | 3: 27481652 | 13452 | 3: 27642990 | 13454 | 3: 27663059 |
| 13458 | 3: 27753499 | 13459 | 3: 27756989 | 13463 | 3: 27871144 | 13465 | 3: 27916123 | 13468 | 3: 27942850 |
| 13469 | 3: 27942868 | 13472 | 3: 27986412 | 13473 | 3: 27990865 | 13476 | 3: 28061404 | 13495 | 3: 28713706 |
| 13509 | 3: 29019861 | 13537 | 3: 29488699 | 13545 | 3: 29585223 | 13546 | 3: 29592827 | 13547 | 3: 29597761 |
| 13548 | 3: 29601864 | 13550 | 3: 29625965 | 13555 | 3: 29681833 | 13558 | 3: 29705190 | 13559 | 3: 29705342 |
| 13560 | 3: 29705512 | 13561 | 3: 29706025 | 13562 | 3: 29706295 | 13563 | 3: 29706336 | 13568 | 3: 29857727 |
| 13570 | 3: 29915442 | 13571 | 3: 29944912 | 13573 | 3: 29954086 | 13575 | 3: 29989198 | 13588 | 3: 30363529 |
| 13607 | 3: 30832758 | 13613 | 3: 30890540 | 13619 | 3: 31013256 | 13622 | 3: 31065769 | 13627 | 3: 31222103 |
| 13630 | 3: 31343441 | 13645 | 3: 31640573 | 13646 | 3: 31689225 | 13652 | 3: 31856497 | 13654 | 3: 31864015 |
| 13655 | 3: 31867053 | 13657 | 3: 31896363 | 13664 | 3: 32046129 | 13665 | 3: 32047685 | 13666 | 3: 32079966 |
| 13668 | 3: 32103555 | 13671 | 3: 32156925 | 13673 | 3: 32164330 | 13676 | 3: 32278723 | 13681 | 3: 32512486 |
| 13699 | 3: 32780141 | 13738 | 3: 32985901 | 13743 | 3: 33003020 | 13762 | 3: 33254753 | 13780 | 3: 33545907 |
| 13781 | 3: 33549574 | 13784 | 3: 33581206 | 13786 | 3: 33623020 | 13787 | 3: 33626853 | 13788 | 3: 33628444 |
| 13789 | 3: 33635163 | 13790 | 3: 33641481 | 13791 | 3: 33644276 | 13792 | 3: 33647252 | 13793 | 3: 33648187 |
| 13794 | 3: 33652206 | 13795 | 3: 33655047 | 13796 | 3: 33667890 | 13797 | 3: 33668793 | 13798 | 3: 33673078 |
| 13799 | 3: 33679729 | 13800 | 3: 33680460 | 13806 | 3: 33800750 | 13810 | 3: 33917904 | 13812 | 3: 33969180 |
| 13814 | 3: 33975547 | 13819 | 3: 34094670 | 13828 | 3: 34304343 | 13832 | 3: 34398092 | 13839 | 3: 34520818 |
| 13840 | 3: 34541029 | 13841 | 3: 34544910 | 13844 | 3: 34626494 | 13845 | 3: 34639336 | 13846 | 3: 34639367 |
| 13847 | 3: 34639414 | 13848 | 3: 34643371 | 13849 | 3: 34644168 | 13850 | 3: 34644227 | 13851 | 3: 34644230 |
| 13852 | 3: 34644292 | 13853 | 3: 34644311 | 13854 | 3: 34646033 | 13855 | 3: 34648675 | 13856 | 3: 34648858 |
| 13857 | 3: 34654205 | 13858 | 3: 34702016 | 13862 | 3: 34749792 | 13865 | 3: 34794868 | 13871 | 3: 34932689 |
| 13872 | 3: 34999359 | 13880 | 3: 35139460 | 13881 | 3: 35139484 | 13886 | 3: 35172154 | 13889 | 3: 35229235 |
| 13892 | 3: 35308462 | 13902 | 3: 35598523 | 13905 | 3: 35702543 | 13906 | 3: 35714205 | 13910 | 3: 35853066 |
| 13914 | 3: 35918054 | 13919 | 3: 36000811 | 13923 | 3: 36106918 | 13924 | 3: 36139522 | 13930 | 3: 36276403 |
| 13937 | 3: 36323043 | 13940 | 3: 36402532 | 13941 | 3: 36414305 | 13950 | 3: 36709252 | 13951 | 3: 36709316 |
| 13958 | 3: 36906114 | 13985 | 3: 37656931 | 13986 | 3: 37657062 | 13991 | 3: 37904335 | 14029 | 3: 39458322 |
| 14043 | 3: 39836580 | 14050 | 3: 40045551 | 14052 | 3: 40104674 | 14057 | 3: 40188225 | 14075 | 3: 40698452 |
| 14081 | 3: 41015843 | 14084 | 3: 41043374 | 14088 | 3: 41141556 | 14093 | 3: 41367055 | 14097 | 3: 41491123 |
| 14100 | 3: 41527680 | 14101 | 3: 41534229 | 14103 | 3: 41589506 | 14104 | 3: 41589571 | 14107 | 3: 41614043 |
| 14113 | 3: 41934611 | 14114 | 3: 41939570 | 14115 | 3: 41940253 | 14117 | 3: 41944095 | 14118 | 3: 41945371 |
| 14119 | 3: 41946261 | 14124 | 3: 42082768 | 14136 | 3: 42496233 | 14137 | 3: 42502863 | 14140 | 3: 42614221 |
| 14141 | 3: 42635738 | 14143 | 3: 42650352 | 14144 | 3: 42659935 | 14148 | 3: 42725238 | 14152 | 3: 42769882 |
| 14155 | 3: 42840302 | 14156 | 3: 42872218 | 14159 | 3: 42900902 | 14160 | 3: 42978663 | 14165 | 3: 43002941 |
| 14167 | 3: 43038774 | 14175 | 3: 43325595 | 14176 | 3: 43325725 | 14177 | 3: 43334230 | 14182 | 3: 43389979 |
| 14183 | 3: 43397421 | 14185 | 3: 43426451 | 14193 | 3: 43680161 | 14194 | 3: 43696024 | 14195 | 3: 43698262 |
| 14196 | 3: 43698948 | 14203 | 3: 43873693 | 14206 | 3: 44017413 | 14215 | 3: 44305690 | 14226 | 3: 44620511 |
| 14235 | 3: 44902917 | 14238 | 3: 44948682 | 14260 | 3: 45663768 | 14298 | 3: 46955918 | 14299 | 3: 46978051 |
| 14309 | 3: 47252028 | 14315 | 3: 47485150 | 14333 | 3: 47811507 | 14352 | 3: 48155117 | 14353 | 3: 48159568 |
| 14363 | 3: 48456906 | 14367 | 3: 48577665 | 14372 | 3: 48681437 | 14374 | 3: 48741921 | 14385 | 3: 48923622 |
| 14390 | 3: 48954466 | 14403 | 3: 49283986 | 14413 | 3: 49441533 | 14414 | 3: 49442661 | 14417 | 3: 49485613 |
| 14418 | 3: 49491032 | 14419 | 3: 49499289 | 14474 | 3: 49732105 | 14477 | 3: 49835535 | 14478 | 3: 49879024 |
| 14479 | 3: 49897610 | 14501 | 3: 50633952 | 14516 | 3: 51122225 | 14527 | 3: 51610699 | 14530 | 3: 51645901 |
| 14532 | 3: 51683713 | 14534 | 3: 51740729 | 14535 | 3: 51755409 | 14603 | 3: 53343252 | 14616 | 3: 53703853 |
| 14626 | 3: 54144822 | 14628 | 3: 54149746 | 14629 | 3: 54167611 | 14630 | 3: 54178592 | 14631 | 3: 54185471 |
| 14632 | 3: 54186364 | 14633 | 3: 54188660 | 14634 | 3: 54201062 | 14638 | 3: 54367531 | 14649 | 3: 55014493 |
| 14650 | 3: 55022327 | 14708 | 3: 56670745 | 14714 | 3: 56924360 | 14716 | 3: 56934727 | 14717 | 3: 56936672 |
| 14719 | 3: 56953053 | 14721 | 3: 56999586 | 14723 | 3: 57032150 | 14727 | 3: 57245572 | 14745 | 3: 57799143 |
| 14749 | 3: 57886444 | 14757 | 3: 58161126 | 14767 | 3: 58455621 | 14770 | 3: 58489145 | 14772 | 3: 58507559 |
| 14776 | 3: 58545248 | 14787 | 3: 58913251 | 14791 | 3: 59017336 | 14795 | 3: 59162716 | 14796 | 3: 59207859 |
| 14816 | 3: 59758400 | 14821 | 3: 60026204 | 14826 | 3: 60180066 | 14831 | 3: 60261618 | 14849 | 3: 60625549 |
| 14858 | 3: 60737754 | 14867 | 3: 60914592 | 14869 | 3: 60962012 | 14888 | 3: 61496668 | 14893 | 3: 61744158 |
| 14896 | 3: 61858795 | 14904 | 3: 62095195 | 14910 | 3: 62278399 | 14914 | 3: 62359529 | 14933 | 3: 62854439 |
| 14945 | 3: 63290027 | 14972 | 3: 63993039 | 14976 | 3: 64069515 | 14991 | 3: 64651171 | 14995 | 3: 64790813 |
| 15006 | 3: 65095548 | 15008 | 3: 65157057 | 15028 | 3: 65566921 | 15032 | 3: 65663032 | 15057 | 3: 66345116 |
| 15058 | 3: 66351139 | 15061 | 3: 66398657 | 15066 | 3: 66502134 | 15070 | 3: 66529229 | 15078 | 3: 66637168 |
| 15083 | 3: 66838529 | 15089 | 3: 66948480 | 15091 | 3: 66974396 | 15092 | 3: 66999361 | 15093 | 3: 67031006 |
| 15095 | 3: 67168089 | 15102 | 3: 67318712 | 15109 | 3: 67473906 | 15116 | 3: 67670465 | 15134 | 3: 68047509 |
| 15140 | 3: 68228132 | 15141 | 3: 68228231 | 15142 | 3: 68276086 | 15143 | 3: 68302197 | 15145 | 3: 68316834 |
| 15149 | 3: 68422911 | 15151 | 3: 68443638 | 15156 | 3: 68551519 | 15158 | 3: 68594137 | 15165 | 3: 68727455 |
| 15166 | 3: 68788461 | 15173 | 3: 68973246 | 15174 | 3: 69004319 | 15177 | 3: 69066161 | 15184 | 3: 69392637 |
| 15226 | 3: 70427777 | 15229 | 3: 70602082 | 15230 | 3: 70652398 | 15255 | 3: 71704029 | 15272 | 3: 72307570 |
| 15273 | 3: 72307597 | 15294 | 3: 73250579 | 15305 | 3: 73571608 | 15309 | 3: 73698701 | 15310 | 3: 73698721 |
| 15316 | 3: 73904881 | 15317 | 3: 73909382 | 15324 | 3: 74202751 | 15327 | 3: 74264677 | 15345 | 3: 74829928 |
| 15349 | 3: 74963727 | 15355 | 3: 75071726 | 15364 | 3: 75370499 | 15370 | 3: 75506540 | 15374 | 3: 75649317 |
| 15375 | 3: 75649320 | 15376 | 3: 75649336 | 15377 | 3: 75649383 | 15398 | 3: 76298830 | 15406 | 3: 76468869 |
| 15415 | 3: 76956223 | 15417 | 3: 77030221 | 15423 | 3: 77162998 | 15425 | 3: 77178882 | 15434 | 3: 77402844 |
| 15435 | 3: 77409935 | 15438 | 3: 77489908 | 15441 | 3: 77571521 | 15449 | 3: 77850286 | 15459 | 3: 78097452 |
| 15465 | 3: 78290774 | 15468 | 3: 78420363 | 15473 | 3: 78568977 | 15474 | 3: 78569227 | 15475 | 3: 78569835 |
| 15478 | 3: 78642264 | 15479 | 3: 78662366 | 15481 | 3: 78785400 | 15489 | 3: 79003182 | 15491 | 3: 79007539 |
| 15497 | 3: 79179242 | 15510 | 3: 79464743 | 15523 | 3: 79956010 | 15525 | 3: 79973859 | 15531 | 3: 80163495 |
| 15537 | 3: 80491657 | 15540 | 3: 80549623 | 15548 | 3: 80733840 | 15556 | 3: 80952119 | 15557 | 3: 80954660 |
| 15558 | 3: 80954761 | 15560 | 3: 80999275 | 15566 | 3: 81103725 | 15578 | 3: 81314640 | 15580 | 3: 81343738 |
| 15582 | 3: 81436844 | 15584 | 3: 81464927 | 15597 | 3: 81749835 | 15613 | 3: 82402375 | 15616 | 3: 82454574 |
| 15620 | 3: 82503792 | 15655 | 3: 83636824 | 15658 | 3: 83652637 | 15673 | 3: 84036094 | 15680 | 3: 84195762 |
| 15694 | 3: 84534786 | 15695 | 3: 84550534 | 15697 | 3: 84560190 | 15726 | 3: 84721616 | 15747 | 3: 85077748 |
| 15751 | 3: 85284944 | 15755 | 3: 85429614 | 15756 | 3: 85429668 | 15757 | 3: 85429696 | 15775 | 3: 85848368 |
| 15783 | 3: 85978048 | 15791 | 3: 86156348 | 15797 | 3: 86290419 | 15800 | 3: 86312689 | 15801 | 3: 86312736 |
| 15805 | 3: 86366796 | 15811 | 3: 86584920 | 15815 | 3: 86652496 | 15827 | 3: 86872323 | 15840 | 3: 87228581 |
| 15843 | 3: 87295571 | 15847 | 3: 87349602 | 15848 | 3: 87379701 | 15929 | 3: 88997759 | 15930 | 3: 89044393 |
| 15932 | 3: 89092242 | 15936 | 3: 89255790 | 15957 | 3: 89639867 | 15963 | 3: 89717766 | 15964 | 3: 89722428 |
| 15966 | 3: 89773983 | 15968 | 3: 89792849 | 15971 | 3: 89858003 | 15972 | 3: 89858025 | 15973 | 3: 89861756 |
| 15974 | 3: 89888858 | 15979 | 3: 89947137 | 15983 | 3: 90125909 | 15987 | 3: 90218397 | 15996 | 3: 90458410 |
| 15998 | 3: 90490960 | 16012 | 3: 90774079 | 16022 | 3: 90916704 | 16028 | 3: 91085209 | 16032 | 3: 91177765 |
| 16038 | 3: 91342742 | 16039 | 3: 91349417 | 16047 | 3: 91577320 | 16050 | 3: 91662702 | 16056 | 3: 91779733 |
| 16057 | 3: 91821560 | 16058 | 3: 91850874 | 16061 | 3: 91944104 | 16064 | 3: 92012709 | 16066 | 3: 92056509 |
| 16072 | 3: 92226650 | 16083 | 3: 92532036 | 16092 | 3: 92747667 | 16093 | 3: 92757686 | 16096 | 3: 92813097 |
| 16103 | 3: 92955459 | 16105 | 3: 92979991 | 16112 | 3: 93070031 | 16115 | 3: 93079352 | 16117 | 3: 93087724 |
| 16123 | 3: 93219307 | 16124 | 3: 93219814 | 16125 | 3: 93220543 | 16126 | 3: 93226524 | 16127 | 3: 93235948 |
| 16128 | 3: 93314047 | 16130 | 3: 93345899 | 16143 | 3: 93782247 | 16144 | 3: 93782331 | 16145 | 3: 93782436 |
| 16159 | 3: 94060442 | 16165 | 3: 94226523 | 16178 | 3: 94716721 | 16180 | 3: 94726765 | 16183 | 3: 94823429 |
| 16184 | 3: 94866313 | 16186 | 3: 94911090 | 16188 | 3: 94976568 | 16190 | 3: 95036766 | 16230 | 3: 96202292 |
| 16238 | 3: 96579309 | 16242 | 3: 96636004 | 16247 | 3: 96785820 | 16248 | 3: 96830575 | 16254 | 3: 96975581 |
| 16260 | 3: 97123954 | 16269 | 3: 97365473 | 16274 | 3: 97457441 | 16275 | 3: 97473661 | 16278 | 3: 97526789 |
| 16290 | 3: 97708909 | 16301 | 3: 97930159 | 16305 | 3: 98116083 | 16309 | 3: 98295744 | 16313 | 3: 98349294 |
| 16317 | 3: 98403571 | 16321 | 3: 98561508 | 16335 | 3: 98923450 | 16337 | 3: 98978448 | 16338 | 3: 98978458 |
| 16348 | 3: 99342353 | 16350 | 3: 99349479 | 16351 | 3: 99356345 | 16352 | 3: 99360354 | 16353 | 3: 99377234 |
| 16358 | 3: 99519922 | 16368 | 3: 99751257 | 16369 | 3: 99765693 | 16370 | 3: 99771380 | 16371 | 3: 99805386 |
| 16372 | 3: 99811235 | 16373 | 3: 99811520 | 16374 | 3: 99818061 | 16375 | 3: 99831451 | 16376 | 3: 99840460 |
| 16377 | 3: 99840480 | 16378 | 3: 99884150 | 16381 | 3: 99928886 | 16383 | 3: 99953595 | 16387 | 3: 100006117 |
| 16388 | 3: 100006731 | 16391 | 3: 100135156 | 16393 | 3: 100148664 | 16403 | 3: 100386736 | 16405 | 3: 100413711 |
| 16406 | 3: 100417680 | 16407 | 3: 100429883 | 16410 | 3: 100503760 | 16411 | 3: 100519527 | 16412 | 3: 100527924 |
| 16413 | 3: 100532091 | 16415 | 3: 100578191 | 16417 | 3: 100604376 | 16418 | 3: 100616906 | 16420 | 3: 100649238 |
| 16421 | 3: 100655322 | 16422 | 3: 100660031 | 16425 | 3: 100706971 | 16426 | 3: 100710303 | 16435 | 3: 100839974 |
| 16440 | 3: 101045558 | 16449 | 3: 101184420 | 16456 | 3: 101301924 | 16457 | 3: 101328497 | 16462 | 3: 101381082 |
| 16496 | 3: 102252547 | 16500 | 3: 102323975 | 16502 | 3: 102346918 | 16504 | 3: 102360619 | 16505 | 3: 102430768 |
| 16506 | 3: 102432444 | 16515 | 3: 102664478 | 16516 | 3: 102684885 | 16517 | 3: 102684933 | 16518 | 3: 102684985 |
| 16520 | 3: 102736091 | 16521 | 3: 102753266 | 16524 | 3: 102804450 | 16529 | 3: 102845961 | 16530 | 3: 102846036 |
| 16531 | 3: 102853282 | 16536 | 3: 102881866 | 16555 | 3: 103364021 | 16560 | 3: 103438363 | 16564 | 3: 103569295 |
| 16566 | 3: 103617298 | 16567 | 3: 103655132 | 16569 | 3: 103692819 | 16571 | 3: 103732748 | 16573 | 3: 103756143 |
| 16578 | 3: 103836979 | 16581 | 3: 103923977 | 16583 | 3: 104007365 | 16584 | 3: 104022881 | 16590 | 3: 104089157 |
| 16598 | 3: 104333611 | 16610 | 3: 104577770 | 16612 | 3: 104588926 | 16614 | 3: 104626150 | 16616 | 3: 104664666 |
| 16618 | 3: 104700803 | 16621 | 3: 104722133 | 16622 | 3: 104733085 | 16624 | 3: 104818821 | 16629 | 3: 104879046 |
| 16641 | 3: 105195217 | 16642 | 3: 105195508 | 16644 | 3: 105197594 | 16645 | 3: 105207363 | 16648 | 3: 105223366 |
| 16649 | 3: 105224129 | 16650 | 3: 105224197 | 16656 | 3: 105344373 | 16657 | 3: 105346169 | 16658 | 3: 105386965 |
| 16659 | 3: 105397156 | 16660 | 3: 105399284 | 16661 | 3: 105401478 | 16664 | 3: 105416679 | 16665 | 3: 105419956 |
| 16666 | 3: 105420955 | 16668 | 3: 105522467 | 16669 | 3: 105528154 | 16670 | 3: 105530865 | 16671 | 3: 105550247 |
| 16672 | 3: 105553161 | 16673 | 3: 105557267 | 16675 | 3: 105587522 | 16676 | 3: 105587615 | 16677 | 3: 105588435 |
| 16678 | 3: 105589110 | 16679 | 3: 105589417 | 16680 | 3: 105589499 | 16681 | 3: 105589515 | 16682 | 3: 105589567 |
| 16683 | 3: 105589840 | 16686 | 3: 105615307 | 16687 | 3: 105623790 | 16688 | 3: 105633378 | 16689 | 3: 105633804 |
| 16691 | 3: 105644182 | 16692 | 3: 105654662 | 16693 | 3: 105674940 | 16696 | 3: 105745106 | 16701 | 3: 105803674 |
| 16702 | 3: 105805027 | 16705 | 3: 105897358 | 16706 | 3: 105897511 | 16707 | 3: 105897845 | 16708 | 3: 105897970 |
| 16709 | 3: 105908966 | 16710 | 3: 105909058 | 16712 | 3: 105935499 | 16713 | 3: 105956333 | 16717 | 3: 106020186 |
| 16724 | 3: 106135737 | 16731 | 3: 106244085 | 16735 | 3: 106337682 | 16745 | 3: 106561725 | 16746 | 3: 106561875 |
| 16747 | 3: 106585197 | 16748 | 3: 106591729 | 16754 | 3: 106655656 | 16755 | 3: 106668839 | 16765 | 3: 106845702 |
| 16771 | 3: 107103522 | 16783 | 3: 107354102 | 16784 | 3: 107375067 | 16785 | 3: 107375088 | 16786 | 3: 107375142 |
| 16789 | 3: 107445270 | 16791 | 3: 107477138 | 16800 | 3: 107604142 | 16807 | 3: 107789881 | 16817 | 3: 107937893 |
| 16819 | 3: 107940715 | 16830 | 3: 108203613 | 16831 | 3: 108224103 | 16833 | 3: 108305450 | 16835 | 3: 108345650 |
| 16850 | 3: 108755292 | 16851 | 3: 108756407 | 16852 | 3: 108760658 | 16853 | 3: 108761540 | 16854 | 3: 108763978 |
| 16855 | 3: 108764835 | 16856 | 3: 108773793 | 16857 | 3: 108777623 | 16858 | 3: 108778270 | 16860 | 3: 108788102 |
| 16861 | 3: 108817854 | 16862 | 3: 108824614 | 16864 | 3: 108842377 | 16885 | 3: 109426329 | 16888 | 3: 109430792 |
| 16889 | 3: 109430841 | 16899 | 3: 109606532 | 16900 | 3: 109656896 | 16916 | 3: 109969045 | 6920 | 3: 110020722 |
| 16921 | 3: 110026915 | 16923 | 3: 110036182 | 16927 | 3: 110153404 | 16946 | 3: 110780122 | 16947 | 3: 110781851 |
| 16948 | 3: 110783875 | 16949 | 3: 110784912 | 16950 | 3: 110786097 | 16954 | 3: 110849640 | 16978 | 3: 111418313 |
| 16986 | 3: 111526183 | 16989 | 3: 111580049 | 17003 | 3: 111889624 | 17019 | 3: 112192839 | 17025 | 3: 112323001 |
| 17026 | 3: 112345897 | 17032 | 3: 112397006 | 17046 | 3: 112761486 | 17047 | 3: 112761940 | 17050 | 3: 112794864 |
| 17058 | 3: 113036203 | 17062 | 3: 113127829 | 17063 | 3: 113131422 | 17064 | 3: 113146141 | 17072 | 3: 113331205 |
| 17073 | 3: 113331220 | 17074 | 3: 113337934 | 17075 | 3: 113341660 | 17078 | 3: 113407286 | 17081 | 3: 113453006 |
| 17083 | 3: 113518551 | 17084 | 3: 113518636 | 17085 | 3: 113524422 | 17089 | 3: 113563964 | 17090 | 3: 113563979 |
| 17091 | 3: 113563997 | 17092 | 3: 113564006 | 17093 | 3: 113564076 | 17094 | 3: 113564096 | 17095 | 3: 113564107 |
| 17096 | 3: 113585771 | 17097 | 3: 113585795 | 17098 | 3: 113585799 | 17100 | 3: 113586101 | 17101 | 3: 113586251 |
| 17102 | 3: 113590170 | 17120 | 3: 114115462 | 17126 | 3: 114165101 | 17137 | 3: 114384157 | 17141 | 3: 114446007 |
| 17144 | 3: 114496084 | 17146 | 3: 114508185 | 17149 | 3: 114543461 | 17150 | 3: 114548928 | 17152 | 3: 114594768 |
| 17156 | 3: 114618765 | 17160 | 3: 114704691 | 17164 | 3: 114748408 | 17175 | 3: 114802567 | 17185 | 3: 114881902 |
| 17190 | 3: 115000401 | 17193 | 3: 115052234 | 17199 | 3: 115107550 | 17200 | 3: 115120882 | 17208 | 3: 115363288 |
| 17210 | 3: 115374146 | 17213 | 3: 115397893 | 17222 | 3: 115484538 | 17225 | 3: 115543657 | 17227 | 3: 115544750 |
| 17231 | 3: 115602232 | 17232 | 3: 115602518 | 17237 | 3: 115733885 | 17238 | 3: 115814948 | 17243 | 3: 115936238 |
| 17246 | 3: 115967688 | 17262 | 3: 116221838 | 17264 | 3: 116272992 | 17270 | 3: 116347067 | 17282 | 3: 116562510 |
| 17291 | 3: 116772922 | 17299 | 3: 116861866 | 17310 | 3: 116928313 | 17314 | 3: 116981402 | 17315 | 3: 116981583 |
| 17316 | 3: 116981638 | 17320 | 3: 117000919 | 17323 | 3: 117031812 | 17324 | 3: 117049753 | 17326 | 3: 117070587 |
| 17330 | 3: 117123078 | 17331 | 3: 117126904 | 17334 | 3: 117149901 | 17337 | 3: 117209899 | 17338 | 3: 117209960 |
| 17341 | 3: 117278310 | 17342 | 3: 117283114 | 17344 | 3: 117317452 | 17345 | 3: 117325008 | 17353 | 3: 117578080 |
| 17360 | 3: 117649528 | 17361 | 3: 117649537 | 17362 | 3: 117662078 | 17365 | 3: 117673624 | 17366 | 3: 117696207 |
| 17370 | 3: 117709813 | 17394 | 3: 118128778 | 17401 | 3: 118223118 | 17403 | 3: 118288926 | 17404 | 3: 118289376 |
| 17405 | 3: 118289408 | 17406 | 3: 118289679 | 17411 | 3: 118387538 | 17420 | 3: 118548276 | 17426 | 3: 118615619 |
| 17436 | 3: 118755947 | 17440 | 3: 118806132 | 17449 | 3: 118883045 | 17457 | 3: 118964562 | 17461 | 3: 119094347 |
| 17467 | 3: 119427638 | 17472 | 3: 119648530 | 17475 | 3: 119673122 | 17476 | 3: 119709097 | 17481 | 3: 119787932 |
| 17486 | 3: 119868121 | 17499 | 3: 120268455 | 17502 | 3: 120306744 | 17503 | 3: 120306964 | 17510 | 3: 120576521 |
| 17515 | 3: 120695918 | 17522 | 3: 120790650 | 17553 | 4: 1446328 | 17648 | 4: 4173794 | 17649 | 4: 4184943 |
| 17663 | 4: 4533982 | 17666 | 4: 4582744 | 17674 | 4: 4681609 | 17675 | 4: 4681672 | 17679 | 4: 4733530 |
| 17686 | 4: 4818261 | 17691 | 4: 4871624 | 17692 | 4: 4931247 | 17693 | 4: 4943634 | 17701 | 4: 5077162 |
| 17703 | 4: 5134591 | 17708 | 4: 5202035 | 17714 | 4: 5319445 | 17715 | 4: 5319859 | 17721 | 4: 5377091 |
| 17722 | 4: 5395548 | 17724 | 4: 5422821 | 17725 | 4: 5437743 | 17726 | 4: 5441832 | 17756 | 4: 6207303 |
| 17787 | 4: 7009102 | 17788 | 4: 7009111 | 17792 | 4: 7073207 | 17793 | 4: 7106157 | 17799 | 4: 7247371 |
| 17808 | 4: 7354530 | 17814 | 4: 7445669 | 17820 | 4: 7632416 | 17821 | 4: 7658763 | 17822 | 4: 7658787 |
| 17823 | 4: 7658831 | 17824 | 4: 7658948 | 17825 | 4: 7658962 | 17827 | 4: 7697381 | 17835 | 4: 7933911 |
| 17836 | 4: 7945782 | 17837 | 4: 7996659 | 17840 | 4: 8046313 | 17844 | 4: 8179768 | 17847 | 4: 8246510 |
| 17850 | 4: 8272646 | 17853 | 4: 8420098 | 17854 | 4: 8435396 | 17856 | 4: 8445348 | 17859 | 4: 8506844 |
| 17883 | 4: 9151646 | 17891 | 4: 9271969 | 17893 | 4: 9323578 | 17896 | 4: 9385962 | 17898 | 4: 9407447 |
| 17900 | 4: 9444470 | 17901 | 4: 9474683 | 17902 | 4: 9477621 | 17904 | 4: 9485074 | 17905 | 4: 9486301 |
| 17906 | 4: 9488068 | 17907 | 4: 9490132 | 17908 | 4: 9490936 | 17909 | 4: 9492909 | 17920 | 4: 9817295 |
| 17949 | 4: 10163330 | 17953 | 4: 10231259 | 17963 | 4: 10387935 | 17973 | 4: 10679109 | 17996 | 4: 11190947 |
| 17997 | 4: 11191071 | 18000 | 4: 11265836 | 18001 | 4: 11269876 | 18002 | 4: 11271703 | 18007 | 4: 11332982 |
| 18008 | 4: 11339823 | 18009 | 4: 11344256 | 18010 | 4: 11344907 | 18029 | 4: 11936615 | 18050 | 4: 12576634 |
| 18052 | 4: 12673015 | 18061 | 4: 12802609 | 18063 | 4: 12826295 | 18066 | 4: 12860940 | 18072 | 4: 13010035 |
| 18091 | 4: 13579464 | 18092 | 4: 13579504 | 18093 | 4: 13579532 | 18098 | 4: 13707039 | 18102 | 4: 13801041 |
| 18106 | 4: 14002326 | 18110 | 4: 14035164 | 18111 | 4: 14061507 | 18112 | 4: 14079747 | 18148 | 4: 15337479 |
| 18153 | 4: 15469076 | 18154 | 4: 15470050 | 18160 | 4: 15586640 | 18182 | 4: 16245146 | 18200 | 4: 16761268 |
| 18210 | 4: 17040967 | 18221 | 4: 17237895 | 18235 | 4: 17572154 | 18236 | 4: 17572159 | 18237 | 4: 17572202 |
| 18238 | 4: 17572213 | 18256 | 4: 18084841 | 18272 | 4: 18585854 | 18277 | 4: 18655237 | 18279 | 4: 18662663 |
| 18280 | 4: 18669066 | 18282 | 4: 18685097 | 18284 | 4: 18697683 | 18286 | 4: 18705074 | 18287 | 4: 18707492 |
| 18293 | 4: 18889879 | 18325 | 4: 19569259 | 18363 | 4: 19999023 | 18368 | 4: 20111420 | 18373 | 4: 20255515 |
| 18379 | 4: 20329251 | 18380 | 4: 20329405 | 18397 | 4: 20796684 | 18398 | 4: 20802780 | 18399 | 4: 20804901 |
| 18419 | 4: 21160009 | 18420 | 4: 21160045 | 18422 | 4: 21189572 | 18424 | 4: 21207059 | 18425 | 4: 21213102 |
| 18460 | 4: 22190378 | 18463 | 4: 22352000 | 18469 | 4: 22526571 | 18488 | 4: 22913622 | 18498 | 4: 23160284 |
| 18510 | 4: 23595303 | 18521 | 4: 23872526 | 18523 | 4: 23891166 | 18526 | 4: 23927655 | 18527 | 4: 23946714 |
| 18536 | 4: 24333008 | 18549 | 4: 24650631 | 18554 | 4: 24810069 | 18557 | 4: 24885290 | 18565 | 4: 25097829 |
| 18566 | 4: 25109810 | 18567 | 4: 25127084 | 18583 | 4: 25647682 | 18587 | 4: 25742989 | 18590 | 4: 25907759 |
| 18591 | 4: 25907762 | 18594 | 4: 25924624 | 18609 | 4: 26257023 | 18621 | 4: 26648480 | 18627 | 4: 26786277 |
| 18641 | 4: 27097020 | 18642 | 4: 27127515 | 18654 | 4: 27476944 | 18662 | 4: 27629208 | 18666 | 4: 27698262 |
| 18681 | 4: 28186796 | 18682 | 4: 28217559 | 18686 | 4: 28338304 | 18690 | 4: 28410444 | 18691 | 4: 28434228 |
| 18703 | 4: 28719533 | 18704 | 4: 28738300 | 18712 | 4: 28975908 | 18714 | 4: 29080932 | 18716 | 4: 29142901 |
| 18745 | 4: 30297344 | 18752 | 4: 30430428 | 18753 | 4: 30430450 | 18754 | 4: 30430542 | 18755 | 4: 30432018 |
| 18757 | 4: 30501662 | 18784 | 4: 31141033 | 18790 | 4: 31306787 | 18795 | 4: 31471170 | 18797 | 4: 31535187 |
| 18801 | 4: 31680539 | 18809 | 4: 31817533 | 18810 | 4: 31854356 | 18814 | 4: 31949743 | 18854 | 4: 33211684 |
| 18858 | 4: 33325825 | 18869 | 4: 33588518 | 18873 | 4: 33639372 | 18874 | 4: 33652107 | 18876 | 4: 33739841 |
| 18878 | 4: 33761684 | 18880 | 4: 33789343 | 18887 | 4: 33926324 | 18894 | 4: 34109934 | 18913 | 4: 34771597 |
| 18921 | 4: 35053751 | 18923 | 4: 35162460 | 18934 | 4: 35468668 | 18964 | 4: 36420563 | 18970 | 4: 36597277 |
| 18971 | 4: 36657445 | 18981 | 4: 36944816 | 18996 | 4: 37260138 | 19008 | 4: 37691142 | 19021 | 4: 38080777 |
| 19023 | 4: 38088154 | 19027 | 4: 38196269 | 19029 | 4: 38253124 | 19034 | 4: 38381571 | 19048 | 4: 38785910 |
| 19058 | 4: 39096980 | 19077 | 4: 39839146 | 19079 | 4: 39869029 | 19096 | 4: 40085698 | 19097 | 4: 40100668 |
| 19111 | 4: 40502117 | 19112 | 4: 40545187 | 19120 | 4: 40660943 | 19122 | 4: 40678229 | 19128 | 4: 40850556 |
| 19131 | 4: 40925102 | 19136 | 4: 40978581 | 19137 | 4: 40990306 | 19138 | 4: 40990370 | 19139 | 4: 40990413 |
| 19140 | 4: 40994417 | 19143 | 4: 41012545 | 19144 | 4: 41012589 | 19145 | 4: 41012593 | 19155 | 4: 41242597 |
| 19171 | 4: 42001110 | 19176 | 4: 42079882 | 19179 | 4: 42269505 | 19186 | 4: 42478244 | 19198 | 4: 42862322 |
| 19217 | 4: 43233221 | 19218 | 4: 43233239 | 19219 | 4: 43233296 | 19220 | 4: 43233323 | 19221 | 4: 43233412 |
| 19223 | 4: 43330695 | 19234 | 4: 43684052 | 19242 | 4: 43934934 | 19244 | 4: 43942237 | 19248 | 4: 44063960 |
| 19253 | 4: 44178421 | 19256 | 4: 44256983 | 19257 | 4: 44268015 | 19260 | 4: 44305806 | 19268 | 4: 44547450 |
| 19282 | 4: 45052002 | 19298 | 4: 45602329 | 19311 | 4: 46002303 | 19315 | 4: 46099926 | 19317 | 4: 46129459 |
| 19329 | 4: 46600207 | 19340 | 4: 46830510 | 19345 | 4: 46921985 | 19346 | 4: 46925441 | 19347 | 4: 46925682 |
| 19348 | 4: 46943050 | 19349 | 4: 46943107 | 19350 | 4: 46943131 | 19363 | 4: 47258425 | 19379 | 4: 47479517 |
| 19383 | 4: 47511989 | 19386 | 4: 47569946 | 19397 | 4: 47904922 | 19399 | 4: 48028783 | 19403 | 4: 48241131 |
| 19420 | 4: 48780844 | 19426 | 4: 49017869 | 19428 | 4: 49022872 | 19430 | 4: 49035315 | 19431 | 4: 49035320 |
| 19432 | 4: 49035381 | 19433 | 4: 49035395 | 19434 | 4: 49035467 | 19435 | 4: 49037953 | 19437 | 4: 49045621 |
| 19438 | 4: 49045631 | 19439 | 4: 49045737 | 19440 | 4: 49045753 | 19441 | 4: 49045756 | 19442 | 4: 49045852 |
| 19444 | 4: 49061063 | 19445 | 4: 49062116 | 19447 | 4: 49107209 | 19452 | 4: 49181169 | 19456 | 4: 49269697 |
| 19457 | 4: 49284053 | 19467 | 4: 49523230 | 19478 | 4: 49842196 | 19482 | 4: 49964915 | 19486 | 4: 50057099 |
| 19489 | 4: 50248085 | 19490 | 4: 50345064 | 19496 | 4: 50538062 | 19515 | 4: 51250626 | 19517 | 4: 51303879 |
| 19526 | 4: 51543766 | 19532 | 4: 51713354 | 19581 | 4: 53246820 | 19594 | 4: 53516423 | 19599 | 4: 53675011 |
| 19614 | 4: 54062446 | 19622 | 4: 54498204 | 19634 | 4: 54973210 | 19638 | 4: 55064494 | 19643 | 4: 55358812 |
| 19646 | 4: 55397907 | 19658 | 4: 55841154 | 19659 | 4: 55864590 | 19660 | 4: 55873977 | 19663 | 4: 55914722 |
| 19665 | 4: 55965169 | 19671 | 4: 56042508 | 19674 | 4: 56091771 | 19683 | 4: 56311977 | 19689 | 4: 56597023 |
| 19691 | 4: 56614952 | 19696 | 4: 56795183 | 19706 | 4: 57053471 | 19711 | 4: 57121533 | 19713 | 4: 57264096 |
| 19722 | 4: 57443352 | 19726 | 4: 57549751 | 19727 | 4: 57552140 | 19730 | 4: 57682773 | 19735 | 4: 57810438 |
| 19764 | 4: 58952251 | 19771 | 4: 59151344 | 19778 | 4: 59234606 | 19796 | 4: 59761295 | 19798 | 4: 59797184 |
| 19799 | 4: 59797205 | 19817 | 4: 60407425 | 19823 | 4: 60610913 | 19829 | 4: 60822961 | 19835 | 4: 61004115 |
| 19843 | 4: 61150481 | 19844 | 4: 61176028 | 19845 | 4: 61188646 | 19868 | 4: 61850527 | 19876 | 4: 62043721 |
| 19886 | 4: 62197348 | 19901 | 4: 62758226 | 19902 | 4: 62762359 | 19903 | 4: 62769531 | 19910 | 4: 62994404 |
| 19911 | 4: 63001098 | 19919 | 4: 63185750 | 19922 | 4: 63289063 | 19926 | 4: 63398159 | 19932 | 4: 63595012 |
| 19945 | 4: 63872067 | 19946 | 4: 63887485 | 19948 | 4: 63898910 | 19949 | 4: 63913879 | 19957 | 4: 64206739 |
| 19966 | 4: 64550273 | 19968 | 4: 64596464 | 19969 | 4: 64598653 | 19970 | 4: 64633068 | 19997 | 4: 65579692 |
| 19998 | 4: 65585572 | 20000 | 4: 65586522 | 20004 | 4: 65662118 | 20005 | 4: 65663680 | 20006 | 4: 65663924 |
| 20011 | 4: 65757109 | 20016 | 4: 65871790 | 20018 | 4: 65887212 | 20020 | 4: 65935466 | 20026 | 4: 66031691 |
| 20030 | 4: 66126342 | 20032 | 4: 66167545 | 20040 | 4: 66351372 | 20044 | 4: 66424351 | 20058 | 4: 66948425 |
| 20064 | 4: 67093419 | 20079 | 4: 67478622 | 20089 | 4: 67825161 | 20093 | 4: 67886242 | 20098 | 4: 67941747 |
| 20103 | 4: 68103259 | 20106 | 4: 68130974 | 20112 | 4: 68281786 | 20118 | 4: 68525805 | 20138 | 4: 69316258 |
| 20146 | 4: 69461895 | 20150 | 4: 69590761 | 20153 | 4: 69624135 | 20162 | 4: 69834879 | 20168 | 4: 69953830 |
| 20177 | 4: 70190883 | 20179 | 4: 70206948 | 20181 | 4: 70239423 | 20184 | 4: 70315106 | 20197 | 4: 70629516 |
| 20200 | 4: 70650674 | 20207 | 4: 70942839 | 20208 | 4: 70955859 | 20210 | 4: 71014142 | 20212 | 4: 71044933 |
| 20241 | 4: 71963805 | 20242 | 4: 71995369 | 20247 | 4: 72082867 | 20250 | 4: 72155086 | 20265 | 4: 72831364 |
| 20266 | 4: 72831400 | 20278 | 4: 73141876 | 20280 | 4: 73230353 | 20281 | 4: 73230433 | 20282 | 4: 73230506 |
| 20287 | 4: 73356120 | 20295 | 4: 73677765 | 20320 | 4: 74355629 | 20326 | 4: 74574092 | 20327 | 4: 74575187 |
| 20328 | 4: 74575293 | 20332 | 4: 74690642 | 20336 | 4: 74750397 | 20337 | 4: 74775088 | 20339 | 4: 74788299 |
| 20347 | 4: 74921800 | 20348 | 4: 74931461 | 20349 | 4: 74937132 | 20351 | 4: 74962440 | 20355 | 4: 75035303 |
| 20360 | 4: 75176025 | 20375 | 4: 75585274 | 20376 | 4: 75596693 | 20395 | 4: 75964360 | 20410 | 4: 76300291 |
| 20411 | 4: 76303426 | 20427 | 4: 76654098 | 20428 | 4: 76665449 | 20431 | 4: 76826893 | 20434 | 4: 77005397 |
| 20438 | 4: 77038646 | 20439 | 4: 77114794 | 20440 | 4: 77131669 | 20441 | 4: 77141818 | 20443 | 4: 77158468 |
| 20446 | 4: 77222263 | 20447 | 4: 77232107 | 20448 | 4: 77235523 | 20449 | 4: 77272270 | 20453 | 4: 77462163 |
| 20459 | 4: 77550845 | 20460 | 4: 77550847 | 20461 | 4: 77550961 | 20463 | 4: 77552548 | 20464 | 4: 77552605 |
| 20465 | 4: 77552647 | 20466 | 4: 77562640 | 20467 | 4: 77564937 | 20468 | 4: 77565007 | 20469 | 4: 77565031 |
| 20470 | 4: 77612846 | 20474 | 4: 77703279 | 20484 | 4: 78046476 | 20493 | 4: 78275533 | 20500 | 4: 78501007 |
| 20516 | 4: 79081070 | 20532 | 4: 79524078 | 20534 | 4: 79581997 | 20535 | 4: 79582037 | 20570 | 4: 80210945 |
| 20571 | 4: 80211747 | 20575 | 4: 80312765 | 20577 | 4: 80342472 | 20578 | 4: 80347612 | 20603 | 4: 81058244 |
| 20606 | 4: 81115882 | 20608 | 4: 81125829 | 20612 | 4: 81203512 | 20613 | 4: 81222120 | 20615 | 4: 81291786 |
| 20624 | 4: 81541207 | 20646 | 4: 82094311 | 20656 | 4: 82418721 | 20664 | 4: 82649849 | 20665 | 4: 82649930 |
| 20668 | 4: 82720591 | 20669 | 4: 82720596 | 20670 | 4: 82720610 | 20671 | 4: 82720657 | 20672 | 4: 82720686 |
| 20673 | 4: 82720715 | 20679 | 4: 82824766 | 20696 | 4: 83387242 | 20697 | 4: 83420381 | 20699 | 4: 83506707 |
| 20700 | 4: 83540447 | 20701 | 4: 83605440 | 20725 | 4: 84374337 | 20748 | 4: 84998314 | 20767 | 4: 85529127 |
| 20769 | 4: 85585852 | 20770 | 4: 85589724 | 20771 | 4: 85609908 | 20783 | 4: 85887734 | 20784 | 4: 85902484 |
| 20791 | 4: 86035429 | 20794 | 4: 86107048 | 20805 | 4: 86429759 | 20807 | 4: 86434813 | 20808 | 4: 86453759 |
| 20812 | 4: 86588536 | 20827 | 4: 86869600 | 20831 | 4: 87015587 | 20832 | 4: 87025872 | 20833 | 4: 87026389 |
| 20847 | 4: 87544345 | 20861 | 4: 87862791 | 20869 | 4: 87966655 | 20870 | 4: 87988315 | 20871 | 4: 88003339 |
| 20874 | 4: 88190387 | 20876 | 4: 88214757 | 20877 | 4: 88230965 | 20878 | 4: 88267363 | 20883 | 4: 88482339 |
| 20916 | 4: 89540510 | 20919 | 4: 89600401 | 20920 | 4: 89600435 | 20921 | 4: 89600533 | 20925 | 4: 89717462 |
| 20926 | 4: 89720129 | 20997 | 4: 91292909 | 21000 | 4: 91358381 | 21011 | 4: 91589372 | 21017 | 4: 91792117 |
| 21021 | 4: 91837317 | 21032 | 4: 92071091 | 21034 | 4: 92150036 | 21036 | 4: 92281567 | 21038 | 4: 92292551 |
| 21039 | 4: 92298261 | 21040 | 4: 92299550 | 21041 | 4: 92307140 | 21042 | 4: 92315009 | 21044 | 4: 92351942 |
| 21049 | 4: 92444591 | 21058 | 4: 92540924 | 21062 | 4: 92634658 | 21065 | 4: 92695366 | 21072 | 4: 92893919 |
| 21083 | 4: 93143125 | 21086 | 4: 93270718 | 21088 | 4: 93325674 | 21091 | 4: 93338554 | 21092 | 4: 93343119 |
| 21093 | 4: 93349924 | 21094 | 4: 93350879 | 21095 | 4: 93352485 | 21096 | 4: 93361239 | 21097 | 4: 93361477 |
| 21098 | 4: 93362689 | 21099 | 4: 93368708 | 21100 | 4: 93373657 | 21101 | 4: 93376458 | 21102 | 4: 93388339 |
| 21104 | 4: 93445593 | 21107 | 4: 93470782 | 21114 | 4: 93581039 | 21121 | 4: 93756393 | 21125 | 4: 93956921 |
| 21138 | 4: 94448413 | 21147 | 4: 94698872 | 21148 | 4: 94699823 | 21151 | 4: 94815446 | 21152 | 4: 94833611 |
| 21156 | 4: 94917698 | 21157 | 4: 94918493 | 21169 | 4: 95254763 | 21170 | 4: 95254765 | 21171 | 4: 95254773 |
| 21172 | 4: 95254787 | 21175 | 4: 95294696 | 21178 | 4: 95382163 | 21196 | 4: 95826773 | 21198 | 4: 95871966 |
| 21203 | 4: 95901052 | 21207 | 4: 95975966 | 21221 | 4: 96011612 | 21233 | 4: 96120257 | 21234 | 4: 96121708 |
| 21235 | 4: 96145894 | 21236 | 4: 96192829 | 21237 | 4: 96196621 | 21241 | 4: 96307908 | 21249 | 4: 96528346 |
| 21250 | 4: 96528347 | 21285 | 4: 96829783 | 21359 | 4: 96940681 | 21365 | 4: 96997475 | 21371 | 4: 97113666 |
| 21416 | 4: 97172490 | 21421 | 4: 97266645 | 21444 | 4: 97789995 | 21450 | 4: 98087559 | 21454 | 4: 98210913 |
| 21455 | 4: 98212198 | 21457 | 4: 98226178 | 21458 | 4: 98226699 | 21459 | 4: 98239198 | 21463 | 4: 98381525 |
| 21464 | 4: 98406747 | 21470 | 4: 98545217 | 21472 | 4: 98604946 | 21473 | 4: 98617473 | 21474 | 4: 98640953 |
| 21477 | 4: 98765309 | 21482 | 4: 98852463 | 21483 | 4: 98862279 | 21492 | 4: 99462535 | 21498 | 4: 99648977 |
| 21499 | 4: 99650381 | 21500 | 4: 99652215 | 21509 | 4: 99864130 | 21511 | 4: 99877799 | 21513 | 4: 99884272 |
| 21515 | 4: 99907629 | 21518 | 4: 100010231 | 21521 | 4: 100048120 | 21522 | 4: 100048151 | 21533 | 4: 100235968 |
| 21558 | 4: 100865583 | 21560 | 4: 100891716 | 21564 | 4: 100958472 | 21569 | 4: 101138513 | 21580 | 4: 101273627 |
| 21581 | 4: 101278348 | 21582 | 4: 101283299 | 21583 | 4: 101286906 | 21584 | 4: 101304065 | 21594 | 4: 101562975 |
| 21595 | 4: 101570088 | 21597 | 4: 101615078 | 21599 | 4: 101627868 | 21604 | 4: 101697277 | 21606 | 4: 101718193 |
| 21607 | 4: 101720088 | 21608 | 4: 101796334 | 21609 | 4: 101817565 | 21612 | 4: 102044239 | 21613 | 4: 102053938 |
| 21623 | 4: 102294876 | 21630 | 4: 102465082 | 21631 | 4: 102515585 | 21632 | 4: 102515711 | 21633 | 4: 102515830 |
| 21635 | 4: 102567199 | 21636 | 4: 102567675 | 21637 | 4: 102571979 | 21638 | 4: 102577247 | 21641 | 4: 102679178 |
| 21650 | 4: 102994861 | 21653 | 4: 103066617 | 21656 | 4: 103118208 | 21672 | 4: 103464890 | 21673 | 4: 103466759 |
| 21678 | 4: 103589488 | 21691 | 4: 103984758 | 21705 | 4: 104406614 | 21713 | 4: 104554874 | 21714 | 4: 104639191 |
| 21719 | 4: 104710790 | 21721 | 4: 104731929 | 21726 | 4: 104879688 | 21730 | 4: 104955413 | 21739 | 4: 105212815 |
| 21742 | 4: 105331039 | 21743 | 4: 105332159 | 21747 | 4: 105369148 | 21753 | 4: 105480650 | 21754 | 4: 105480728 |
| 21755 | 4: 105482792 | 21766 | 4: 105750600 | 21767 | 4: 105757195 | 21768 | 4: 105785525 | 21773 | 4: 106025379 |
| 21775 | 4: 106025551 | 21780 | 4: 106081660 | 21781 | 4: 106085160 | 21782 | 4: 106088179 | 21783 | 4: 106088193 |
| 21784 | 4: 106088224 | 21785 | 4: 106088251 | 21786 | 4: 106088286 | 21792 | 4: 106177289 | 21796 | 4: 106243037 |
| 21797 | 4: 106246162 | 21798 | 4: 106257365 | 21807 | 4: 106459877 | 21808 | 4: 106461237 | 21809 | 4: 106461523 |
| 21810 | 4: 106462584 | 21811 | 4: 106463211 | 21812 | 4: 106526851 | 21817 | 4: 106674077 | 21820 | 4: 106723818 |
| 21821 | 4: 106724580 | 21822 | 4: 106738338 | 21823 | 4: 106768265 | 21827 | 4: 106823063 | 21833 | 4: 106915810 |
| 21835 | 4: 106985422 | 21837 | 4: 106991677 | 21838 | 4: 106991678 | 21839 | 4: 106991818 | 21840 | 4: 106991819 |
| 21841 | 4: 106993054 | 21842 | 4: 106993083 | 21843 | 4: 106993086 | 21844 | 4: 106993163 | 21845 | 4: 106993484 |
| 21846 | 4: 106993491 | 21847 | 4: 106993778 | 21848 | 4: 106993951 | 21849 | 4: 106993975 | 21850 | 4: 106994109 |
| 21851 | 4: 106994258 | 21852 | 4: 106997706 | 21853 | 4: 106998207 | 21857 | 4: 107044582 | 21858 | 4: 107070723 |
| 21859 | 4: 107075139 | 21867 | 4: 107202696 | 21868 | 4: 107202723 | 21869 | 4: 107202867 | 21870 | 4: 107202876 |
| 21873 | 4: 107293705 | 21874 | 4: 107293767 | 21875 | 4: 107293937 | 21876 | 4: 107293975 | 21877 | 4: 107293984 |
| 21878 | 4: 107294409 | 21879 | 4: 107294413 | 21884 | 4: 107599054 | 21891 | 4: 107760331 | 21896 | 4: 107893188 |
| 21918 | 4: 108666311 | 21919 | 4: 108666329 | 21936 | 4: 109206944 | 21996 | 4: 110555410 | 21998 | 4: 110627140 |
| 22003 | 4: 110751708 | 22004 | 4: 110751716 | 22005 | 4: 110759035 | 22006 | 4: 110759043 | 22007 | 4: 110759085 |
| 22008 | 4: 110759138 | 22009 | 4: 110759193 | 22012 | 4: 110870537 | 22017 | 4: 110963804 | 22018 | 4: 110997685 |
| 22021 | 4: 111094939 | 22027 | 4: 111132995 | 22030 | 4: 111174187 | 22036 | 4: 111290634 | 22041 | 4: 111393405 |
| 22042 | 4: 111393524 | 22060 | 4: 111696373 | 22072 | 4: 111836637 | 22075 | 4: 111906579 | 22076 | 4: 111910434 |
| 22077 | 4: 111911436 | 22079 | 4: 111972681 | 22085 | 4: 112108098 | 22087 | 4: 112136295 | 22090 | 4: 112153870 |
| 22091 | 4: 112155488 | 22093 | 4: 112172784 | 22097 | 4: 112238690 | 22100 | 4: 112269261 | 22110 | 4: 112424206 |
| 22112 | 4: 112448562 | 22113 | 4: 112452844 | 22117 | 4: 112516104 | 22122 | 4: 112599976 | 22124 | 4: 112652763 |
| 22125 | 4: 112660638 | 22126 | 4: 112663309 | 22127 | 4: 112664440 | 22128 | 4: 112665144 | 22129 | 4: 112671210 |
| 22130 | 4: 112675707 | 22131 | 4: 112676199 | 22132 | 4: 112677340 | 22133 | 4: 112681999 | 22136 | 4: 112716249 |
| 22138 | 4: 112739929 | 22139 | 4: 112740655 | 22140 | 4: 112741810 | 22141 | 4: 112748029 | 22142 | 4: 112749294 |
| 22143 | 4: 112754237 | 22144 | 4: 112755579 | 22145 | 4: 112769671 | 22146 | 4: 112770319 | 22147 | 4: 112785850 |
| 22153 | 4: 112842045 | 22154 | 4: 112844573 | 22155 | 4: 112855696 | 22157 | 4: 112873667 | 22158 | 4: 112873671 |
| 22159 | 4: 112873791 | 22160 | 4: 112874161 | 22162 | 4: 112920884 | 22163 | 4: 112920908 | 22164 | 4: 112958565 |
| 22178 | 4: 113478902 | 22184 | 4: 113579221 | 22185 | 4: 113579506 | 22186 | 4: 113584318 | 22187 | 4: 113584327 |
| 22188 | 4: 113584399 | 22189 | 4: 113584405 | 22190 | 4: 113587147 | 22199 | 4: 113617600 | 22200 | 4: 113617762 |
| 22201 | 4: 113622976 | 22220 | 4: 113685923 | 22221 | 4: 113693137 | 22222 | 4: 113706536 | 22230 | 4: 113862937 |
| 22232 | 4: 113865696 | 22233 | 4: 113865709 | 22234 | 4: 113865711 | 22235 | 4: 113889872 | 22236 | 4: 113924572 |
| 22239 | 4: 113963854 | 22242 | 4: 114103350 | 22247 | 4: 114278681 | 22261 | 4: 114688072 | 22262 | 4: 114690368 |
| 22263 | 4: 114690442 | 22264 | 4: 114691485 | 22265 | 4: 114692365 | 22270 | 4: 114751599 | 22271 | 4: 114758803 |
| 22273 | 4: 114770151 | 22275 | 4: 114805260 | 22276 | 4: 114812473 | 22277 | 4: 114812501 | 22278 | 4: 114812550 |
| 22279 | 4: 114812583 | 22289 | 4: 115031057 | 22290 | 4: 115033034 | 22291 | 4: 115043261 | 22292 | 4: 115066503 |
| 22293 | 4: 115067836 | 22294 | 4: 115071402 | 22295 | 4: 115072381 | 22296 | 4: 115074258 | 22297 | 4: 115075256 |
| 22298 | 4: 115077110 | 22299 | 4: 115077972 | 22300 | 4: 115079711 | 22301 | 4: 115105025 | 22304 | 4: 115127050 |
| 22305 | 4: 115127065 | 22309 | 4: 115153886 | 22310 | 4: 115153896 | 22311 | 4: 115163025 | 22326 | 4: 115413253 |
| 22327 | 4: 115413261 | 22328 | 4: 115413321 | 22329 | 4: 115413386 | 22330 | 4: 115413395 | 22359 | 4: 116192647 |
| 22360 | 4: 116192669 | 22361 | 4: 116192689 | 22362 | 4: 116192738 | 22363 | 4: 116192764 | 22379 | 4: 116445855 |
| 22402 | 4: 116915804 | 22408 | 4: 117000567 | 22409 | 4: 117000602 | 22410 | 4: 117000603 | 22420 | 4: 117210404 |
| 22427 | 4: 117486610 | 22428 | 4: 117505997 | 22448 | 4: 117829938 | 22465 | 4: 118353876 | 22466 | 4: 118358762 |
| 22472 | 4: 118419883 | 22476 | 4: 118517071 | 22485 | 4: 118646844 | 22490 | 4: 118756391 | 22494 | 4: 118829611 |
| 22496 | 4: 118829788 | 22504 | 4: 118939017 | 22523 | 4: 119457121 | 22535 | 4: 119689809 | 22553 | 5: 77074 |
| 22579 | 5: 827590 | 22580 | 5: 834238 | 22581 | 5: 882265 | 22582 | 5: 882349 | 22583 | 5: 882384 |
| 22584 | 5: 903638 | 22597 | 5: 1099972 | 22608 | 5: 1345105 | 22610 | 5: 1356051 | 22611 | 5: 1356065 |
| 22612 | 5: 1356090 | 22613 | 5: 1356140 | 22614 | 5: 1356177 | 22615 | 5: 1356191 | 22618 | 5: 1456630 |
| 22623 | 5: 1589478 | 22625 | 5: 1618964 | 22670 | 5: 2514974 | 22681 | 5: 2735966 | 22684 | 5: 2800666 |
| 22698 | 5: 3349859 | 22699 | 5: 3349891 | 22719 | 5: 4068348 | 22749 | 5: 4663849 | 22757 | 5: 4889807 |
| 22768 | 5: 5162198 | 22770 | 5: 5188536 | 22772 | 5: 5196871 | 22778 | 5: 5266489 | 22780 | 5: 5300800 |
| 22789 | 5: 5528412 | 22792 | 5: 5566952 | 22804 | 5: 5799927 | 22805 | 5: 5817089 | 22814 | 5: 6016548 |
| 22822 | 5: 6285112 | 22826 | 5: 6336054 | 22828 | 5: 6436778 | 22838 | 5: 6632102 | 22859 | 5: 7063231 |
| 22871 | 5: 7268739 | 22873 | 5: 7277360 | 22874 | 5: 7277411 | 22877 | 5: 7377438 | 22895 | 5: 7885365 |
| 22915 | 5: 8557986 | 22928 | 5: 8826886 | 22932 | 5: 8934422 | 22933 | 5: 8934467 | 22934 | 5: 8934474 |
| 22962 | 5: 9693605 | 22964 | 5: 9730547 | 22967 | 5: 9797519 | 22968 | 5: 9803049 | 22969 | 5: 9856996 |
| 22970 | 5: 9857138 | 22971 | 5: 9858180 | 22976 | 5: 9982908 | 22978 | 5: 10011555 | 22980 | 5: 10029875 |
| 22981 | 5: 10034496 | 22989 | 5: 10156211 | 22991 | 5: 10201512 | 22992 | 5: 10252022 | 22993 | 5: 10278854 |
| 23007 | 5: 10506388 | 23014 | 5: 10670341 | 23020 | 5: 10828061 | 23021 | 5: 10843507 | 23022 | 5: 10843627 |
| 23023 | 5: 10844849 | 23031 | 5: 11089678 | 23063 | 5: 12028271 | 23067 | 5: 12102588 | 23068 | 5: 12103176 |
| 23073 | 5: 12156021 | 23074 | 5: 12156678 | 23083 | 5: 12325031 | 23084 | 5: 12326106 | 23096 | 5: 12460715 |
| 23097 | 5: 12473390 | 23105 | 5: 12528945 | 23111 | 5: 12719804 | 23121 | 5: 13036017 | 23124 | 5: 13069760 |
| 23139 | 5: 13541155 | 23151 | 5: 14006938 | 23155 | 5: 14124180 | 23171 | 5: 14599931 | 23172 | 5: 14600013 |
| 23175 | 5: 14680469 | 23195 | 5: 15100766 | 23199 | 5: 15173290 | 23216 | 5: 15462968 | 23219 | 5: 15566954 |
| 23220 | 5: 15566955 | 23221 | 5: 15566965 | 23222 | 5: 15566966 | 23223 | 5: 15566972 | 23224 | 5: 15566977 |
| 23225 | 5: 15567052 | 23226 | 5: 15567070 | 23227 | 5: 15567096 | 23228 | 5: 15567111 | 23232 | 5: 15704626 |
| 23266 | 5: 16709205 | 23270 | 5: 16800463 | 23282 | 5: 17119922 | 23285 | 5: 17174787 | 23288 | 5: 17241349 |
| 23293 | 5: 17398558 | 23306 | 5: 17895598 | 23307 | 5: 17903579 | 23318 | 5: 18353350 | 23321 | 5: 18410507 |
| 23322 | 5: 18410599 | 23338 | 5: 18941613 | 23351 | 5: 19280880 | 23359 | 5: 19568730 | 23374 | 5: 19885896 |
| 23376 | 5: 19976107 | 23377 | 5: 19993960 | 23378 | 5: 20014240 | 23381 | 5: 20097603 | 23382 | 5: 20149313 |
| 23384 | 5: 20195300 | 23389 | 5: 20436830 | 23396 | 5: 20590589 | 23399 | 5: 20732321 | 23405 | 5: 20832737 |
| 23406 | 5: 20832885 | 23407 | 5: 20833047 | 23409 | 5: 20843567 | 23410 | 5: 20910258 | 23411 | 5: 20936220 |
| 23416 | 5: 21019650 | 23422 | 5: 21196220 | 23445 | 5: 21745511 | 23447 | 5: 21797550 | 23452 | 5: 21874789 |
| 23454 | 5: 21920920 | 23466 | 5: 22115062 | 23473 | 5: 22298578 | 23474 | 5: 22323880 | 23477 | 5: 22412419 |
| 23487 | 5: 22513497 | 23491 | 5: 22558133 | 23492 | 5: 22558154 | 23493 | 5: 22558155 | 23502 | 5: 22837489 |
| 23503 | 5: 22837951 | 23507 | 5: 22898017 | 23509 | 5: 22944167 | 23510 | 5: 22947768 | 23514 | 5: 23017065 |
| 23515 | 5: 23045588 | 23517 | 5: 23055987 | 23524 | 5: 23260695 | 23525 | 5: 23261205 | 23526 | 5: 23269298 |
| 23530 | 5: 23386932 | 23533 | 5: 23440852 | 23534 | 5: 23449942 | 23542 | 5: 23608812 | 23545 | 5: 23736650 |
| 23548 | 5: 23753028 | 23552 | 5: 23816911 | 23556 | 5: 23949886 | 23561 | 5: 23975472 | 23570 | 5: 24168834 |
| 23578 | 5: 24352659 | 23579 | 5: 24352754 | 23587 | 5: 24564540 | 23591 | 5: 24706081 | 23594 | 5: 24861511 |
| 23595 | 5: 24861917 | 23596 | 5: 24862040 | 23597 | 5: 24863117 | 23598 | 5: 24864988 | 23599 | 5: 24865092 |
| 23610 | 5: 25042104 | 23618 | 5: 25424142 | 23619 | 5: 25424207 | 23622 | 5: 25531432 | 23627 | 5: 25660326 |
| 23629 | 5: 25668094 | 23632 | 5: 25770487 | 23635 | 5: 25865044 | 23641 | 5: 25948717 | 23642 | 5: 25953449 |
| 23648 | 5: 26274617 | 23652 | 5: 26346656 | 23653 | 5: 26359865 | 23654 | 5: 26372843 | 23655 | 5: 26380803 |
| 23661 | 5: 26513500 | 23662 | 5: 26516803 | 23663 | 5: 26527639 | 23664 | 5: 26531218 | 23672 | 5: 26728680 |
| 23674 | 5: 26750096 | 23679 | 5: 26881116 | 23684 | 5: 27043324 | 23685 | 5: 27062257 | 23686 | 5: 27098658 |
| 23700 | 5: 27397526 | 23705 | 5: 27478824 | 23712 | 5: 27611131 | 23718 | 5: 27732993 | 23724 | 5: 27782046 |
| 23726 | 5: 27821648 | 23730 | 5: 27894859 | 23736 | 5: 28067367 | 23738 | 5: 28076547 | 23741 | 5: 28125774 |
| 23750 | 5: 28398456 | 23754 | 5: 28468312 | 23755 | 5: 28474788 | 23770 | 5: 28759211 | 23771 | 5: 28764621 |
| 23776 | 5: 28916451 | 23779 | 5: 28965670 | 23787 | 5: 29216390 | 23792 | 5: 29377131 | 23809 | 5: 29727121 |
| 23815 | 5: 29862958 | 23819 | 5: 29973188 | 23830 | 5: 30143566 | 23838 | 5: 30392463 | 23844 | 5: 30521361 |
| 23845 | 5: 30526943 | 23846 | 5: 30564929 | 23847 | 5: 30571021 | 23848 | 5: 30583097 | 23849 | 5: 30584287 |
| 23852 | 5: 30656853 | 23861 | 5: 30885641 | 23863 | 5: 30907588 | 23864 | 5: 30945722 | 23869 | 5: 31116777 |
| 23890 | 5: 31693587 | 23899 | 5: 32136613 | 23912 | 5: 32401173 | 23913 | 5: 32414358 | 23916 | 5: 32527988 |
| 23917 | 5: 32544707 | 23918 | 5: 32549914 | 23921 | 5: 32589543 | 23931 | 5: 32826960 | 23939 | 5: 33070073 |
| 23941 | 5: 33080199 | 23951 | 5: 33311326 | 23955 | 5: 33354800 | 23962 | 5: 33573362 | 23971 | 5: 33769971 |
| 23972 | 5: 33803288 | 23979 | 5: 33964757 | 23980 | 5: 33974899 | 24008 | 5: 34894503 | 24010 | 5: 34992883 |
| 24011 | 5: 34992887 | 24012 | 5: 34992888 | 24014 | 5: 35004904 | 24015 | 5: 35004932 | 24016 | 5: 35039187 |
| 24017 | 5: 35055317 | 24018 | 5: 35080229 | 24022 | 5: 35207804 | 24033 | 5: 35577195 | 24036 | 5: 35771527 |
| 24066 | 5: 36566052 | 24079 | 5: 37113142 | 24101 | 5: 37695072 | 24102 | 5: 37711059 | 24104 | 5: 37735001 |
| 24109 | 5: 37845630 | 24113 | 5: 37881472 | 24119 | 5: 37991676 | 24122 | 5: 38029636 | 24123 | 5: 38033300 |
| 24125 | 5: 38058027 | 24127 | 5: 38106278 | 24129 | 5: 38114225 | 24130 | 5: 38116179 | 24131 | 5: 38135398 |
| 24134 | 5: 38174231 | 24144 | 5: 38453061 | 24145 | 5: 38483708 | 24146 | 5: 38527379 | 24147 | 5: 38531595 |
| 24148 | 5: 38544831 | 24150 | 5: 38593359 | 24163 | 5: 39154317 | 24165 | 5: 39228037 | 24166 | 5: 39257315 |
| 24186 | 5: 39750764 | 24202 | 5: 40183299 | 24204 | 5: 40237324 | 24207 | 5: 40321836 | 24208 | 5: 40321842 |
| 24209 | 5: 40321905 | 24210 | 5: 40321940 | 24211 | 5: 40322458 | 24212 | 5: 40322491 | 24214 | 5: 40326831 |
| 24215 | 5: 40340341 | 24216 | 5: 40342525 | 24227 | 5: 40812862 | 24251 | 5: 41504706 | 24264 | 5: 41958537 |
| 24265 | 5: 41958597 | 24266 | 5: 41958622 | 24267 | 5: 41987705 | 24270 | 5: 42145772 | 24282 | 5: 42611303 |
| 24291 | 5: 42843742 | 24296 | 5: 42890909 | 24302 | 5: 43044298 | 24305 | 5: 43082557 | 24314 | 5: 43415290 |
| 24315 | 5: 43480306 | 24318 | 5: 43506882 | 24324 | 5: 43599378 | 24331 | 5: 43811761 | 24334 | 5: 43863604 |
| 24335 | 5: 43864090 | 24336 | 5: 43864115 | 24337 | 5: 43869544 | 24338 | 5: 43875060 | 24339 | 5: 43878846 |
| 24340 | 5: 43880997 | 24341 | 5: 43881099 | 24342 | 5: 43883078 | 24343 | 5: 43897037 | 24344 | 5: 43897095 |
| 24346 | 5: 43901763 | 24350 | 5: 44040614 | 24351 | 5: 44049829 | 24360 | 5: 44388064 | 24374 | 5: 44804564 |
| 24377 | 5: 44906731 | 24384 | 5: 45175795 | 24391 | 5: 45366260 | 24400 | 5: 45549099 | 24403 | 5: 45619287 |
| 24414 | 5: 45917363 | 24422 | 5: 46063715 | 24424 | 5: 46103767 | 24425 | 5: 46103871 | 24435 | 5: 46335844 |
| 24440 | 5: 46443847 | 24442 | 5: 46516496 | 24483 | 5: 47241615 | 24489 | 5: 47478077 | 24492 | 5: 47556051 |
| 24494 | 5: 47596785 | 24507 | 5: 48011805 | 24509 | 5: 48115271 | 24513 | 5: 48219521 | 24521 | 5: 48357494 |
| 24546 | 5: 49071305 | 24547 | 5: 49121969 | 24548 | 5: 49128930 | 24551 | 5: 49244193 | 24555 | 5: 49443860 |
| 24557 | 5: 49480432 | 24576 | 5: 50003215 | 24577 | 5: 50004728 | 24587 | 5: 50241038 | 24593 | 5: 50468141 |
| 24601 | 5: 50729869 | 24604 | 5: 50805474 | 24606 | 5: 50826119 | 24612 | 5: 50987421 | 24613 | 5: 50995228 |
| 24615 | 5: 51012775 | 24616 | 5: 51014179 | 24617 | 5: 51026281 | 24618 | 5: 51028777 | 24619 | 5: 51028823 |
| 24620 | 5: 51045771 | 24621 | 5: 51046494 | 24622 | 5: 51046653 | 24623 | 5: 51050849 | 24624 | 5: 51055590 |
| 24625 | 5: 51056409 | 24626 | 5: 51074757 | 24627 | 5: 51076137 | 24629 | 5: 51106212 | 24630 | 5: 51125457 |
| 24633 | 5: 51222282 | 24639 | 5: 51428229 | 24649 | 5: 51662795 | 24651 | 5: 51737595 | 24652 | 5: 51807509 |
| 24660 | 5: 52059878 | 24661 | 5: 52059908 | 24662 | 5: 52059930 | 24680 | 5: 52526329 | 24693 | 5: 53179786 |
| 24711 | 5: 53766718 | 24712 | 5: 53779538 | 24713 | 5: 53781567 | 24714 | 5: 53786730 | 24717 | 5: 53862521 |
| 24723 | 5: 54013034 | 24732 | 5: 54299758 | 24733 | 5: 54301166 | 24734 | 5: 54304681 | 24735 | 5: 54306088 |
| 24736 | 5: 54309409 | 24737 | 5: 54324570 | 24738 | 5: 54332158 | 24740 | 5: 54358159 | 24741 | 5: 54367083 |
| 24742 | 5: 54370187 | 24743 | 5: 54376482 | 24745 | 5: 54399560 | 24748 | 5: 54511599 | 24749 | 5: 54514698 |
| 24751 | 5: 54563791 | 24755 | 5: 54632888 | 24761 | 5: 54765009 | 24785 | 5: 55509920 | 24789 | 5: 55583411 |
| 24790 | 5: 55619423 | 24791 | 5: 55634685 | 24792 | 5: 55640765 | 24793 | 5: 55652450 | 24795 | 5: 55670462 |
| 24797 | 5: 55696567 | 24798 | 5: 55702652 | 24799 | 5: 55707061 | 24802 | 5: 55873933 | 24803 | 5: 55879578 |
| 24805 | 5: 55940985 | 24807 | 5: 55956661 | 24848 | 5: 56720568 | 24849 | 5: 56792015 | 24853 | 5: 56910201 |
| 24854 | 5: 56953231 | 24859 | 5: 57100134 | 24864 | 5: 57204918 | 24873 | 5: 57427084 | 24882 | 5: 57667264 |
| 24883 | 5: 57672067 | 24896 | 5: 58068683 | 24904 | 5: 58250121 | 24905 | 5: 58285910 | 24915 | 5: 58749685 |
| 24923 | 5: 59304558 | 24925 | 5: 59313596 | 24929 | 5: 59364960 | 24930 | 5: 59365133 | 24941 | 5: 59865035 |
| 24950 | 5: 60107106 | 24956 | 5: 60233686 | 24958 | 5: 60262488 | 24960 | 5: 60297442 | 24962 | 5: 60326833 |
| 24965 | 5: 60380312 | 24971 | 5: 60510721 | 24972 | 5: 60532648 | 24973 | 5: 60543258 | 24975 | 5: 60557011 |
| 24977 | 5: 60595067 | 24983 | 5: 60718806 | 24985 | 5: 60758142 | 24986 | 5: 60766663 | 24987 | 5: 60766858 |
| 24996 | 5: 61052215 | 25025 | 5: 62076373 | 25033 | 5: 62426367 | 25040 | 5: 62518142 | 25043 | 5: 62636512 |
| 25051 | 5: 62933839 | 25052 | 5: 62941170 | 25083 | 5: 63542114 | 25095 | 5: 63850062 | 25104 | 5: 64226221 |
| 25106 | 5: 64234434 | 25118 | 5: 64619628 | 25137 | 5: 65171005 | 25138 | 5: 65183093 | 25142 | 5: 65232220 |
| 25151 | 5: 65446380 | 25154 | 5: 65535107 | 25155 | 5: 65542737 | 25156 | 5: 65543322 | 25157 | 5: 65543372 |
| 25167 | 5: 65703423 | 25168 | 5: 65712111 | 25169 | 5: 65751283 | 25170 | 5: 65752854 | 25172 | 5: 65760426 |
| 25173 | 5: 65761371 | 25179 | 5: 66009583 | 25195 | 5: 66518756 | 25225 | 5: 67447645 | 25226 | 5: 67449152 |
| 25227 | 5: 67463576 | 25228 | 5: 67465088 | 25229 | 5: 67468026 | 25230 | 5: 67470940 | 25231 | 5: 67473722 |
| 25232 | 5: 67475068 | 25233 | 5: 67475725 | 25234 | 5: 67477065 | 25235 | 5: 67481131 | 25237 | 5: 67514487 |
| 25238 | 5: 67526075 | 25239 | 5: 67526864 | 25241 | 5: 67555054 | 25242 | 5: 67563065 | 25243 | 5: 67568345 |
| 25251 | 5: 67846005 | 25252 | 5: 67848289 | 25253 | 5: 67851400 | 25260 | 5: 68044942 | 25261 | 5: 68062320 |
| 25265 | 5: 68180283 | 25267 | 5: 68195298 | 25277 | 5: 68311976 | 25280 | 5: 68341993 | 25282 | 5: 68419217 |
| 25284 | 5: 68527931 | 25292 | 5: 68745462 | 25294 | 5: 68847086 | 25305 | 5: 68998098 | 25309 | 5: 69114492 |
| 25314 | 5: 69191792 | 25315 | 5: 69211669 | 25318 | 5: 69282729 | 25319 | 5: 69295982 | 25327 | 5: 69507467 |
| 25329 | 5: 69534170 | 25330 | 5: 69540325 | 25339 | 5: 69690113 | 25346 | 5: 69807021 | 25354 | 5: 70143301 |
| 25369 | 5: 70657641 | 25371 | 5: 70725053 | 25383 | 5: 70904784 | 25385 | 5: 70931624 | 25388 | 5: 71041333 |
| 25390 | 5: 71113115 | 25393 | 5: 71147255 | 25397 | 5: 71158676 | 25399 | 5: 71204940 | 25401 | 5: 71215860 |
| 25402 | 5: 71247169 | 25404 | 5: 71274477 | 25406 | 5: 71300632 | 25408 | 5: 71346087 | 25409 | 5: 71377241 |
| 25418 | 5: 71535392 | 25422 | 5: 71629712 | 25432 | 5: 71856285 | 25433 | 5: 71893342 | 25434 | 5: 71896515 |
| 25435 | 5: 71909707 | 25438 | 5: 71958333 | 25442 | 5: 72039709 | 25443 | 5: 72058094 | 25445 | 5: 72064330 |
| 25458 | 5: 72293857 | 25461 | 5: 72366005 | 25462 | 5: 72368884 | 25475 | 5: 72696496 | 25500 | 5: 73554593 |
| 25504 | 5: 73672740 | 25521 | 5: 73884415 | 25522 | 5: 73908825 | 25523 | 5: 73980075 | 25529 | 5: 74150522 |
| 25532 | 5: 74225942 | 25533 | 5: 74245072 | 25545 | 5: 74650187 | 25547 | 5: 74725927 | 25549 | 5: 74742101 |
| 25555 | 5: 74908285 | 25556 | 5: 74908338 | 25557 | 5: 74909826 | 25558 | 5: 74911263 | 25559 | 5: 74911341 |
| 25560 | 5: 74915786 | 25561 | 5: 74919175 | 25562 | 5: 74925227 | 25563 | 5: 74925818 | 25564 | 5: 74926028 |
| 25565 | 5: 74928887 | 25566 | 5: 74936574 | 25571 | 5: 75167453 | 25574 | 5: 75224410 | 25575 | 5: 75224450 |
| 25576 | 5: 75224458 | 25577 | 5: 75224463 | 25579 | 5: 75224524 | 25582 | 5: 75261093 | 25586 | 5: 75281166 |
| 25599 | 5: 75676623 | 25605 | 5: 75823353 | 25608 | 5: 75912892 | 25609 | 5: 75919398 | 25614 | 5: 75988734 |
| 25620 | 5: 76209127 | 25621 | 5: 76269564 | 25622 | 5: 76269603 | 25631 | 5: 76640050 | 25632 | 5: 76666195 |
| 25640 | 5: 76784217 | 25649 | 5: 77156529 | 25650 | 5: 77161120 | 25651 | 5: 77180731 | 25652 | 5: 77193464 |
| 25653 | 5: 77193475 | 25654 | 5: 77193500 | 25660 | 5: 77277820 | 25661 | 5: 77302753 | 25662 | 5: 77331990 |
| 25664 | 5: 77340074 | 25666 | 5: 77362757 | 25667 | 5: 77410429 | 25677 | 5: 77928157 | 25678 | 5: 77928784 |
| 25692 | 5: 78324681 | 25698 | 5: 78506395 | 25699 | 5: 78510349 | 25700 | 5: 78522919 | 25708 | 5: 78820352 |
| 25709 | 5: 78820587 | 25710 | 5: 78820588 | 25711 | 5: 78820618 | 25712 | 5: 78820726 | 25713 | 5: 78820795 |
| 25714 | 5: 78820814 | 25715 | 5: 78821054 | 25718 | 5: 78829751 | 25719 | 5: 78832107 | 25720 | 5: 78832141 |
| 25721 | 5: 78832271 | 25752 | 5: 79910151 | 25775 | 5: 80693563 | 25784 | 5: 80982109 | 25788 | 5: 81166934 |
| 25794 | 5: 81369156 | 25802 | 5: 81505086 | 25816 | 5: 82023846 | 25818 | 5: 82073695 | 25822 | 5: 82125171 |
| 25825 | 5: 82161343 | 25835 | 5: 82321735 | 25839 | 5: 82419134 | 25842 | 5: 82485421 | 25843 | 5: 82485429 |
| 25844 | 5: 82491588 | 25847 | 5: 82535021 | 25868 | 5: 82755673 | 25874 | 5: 83018943 | 25879 | 5: 83102062 |
| 25885 | 5: 83210516 | 25887 | 5: 83258916 | 25890 | 5: 83347445 | 25919 | 5: 83920522 | 25924 | 5: 84024864 |
| 26062 | 5: 84271561 | 26081 | 5: 84723773 | 26087 | 5: 84917552 | 26096 | 5: 85106613 | 26097 | 5: 85112695 |
| 26102 | 5: 85286730 | 26138 | 5: 86172964 | 26151 | 5: 86508471 | 26155 | 5: 86538135 | 26167 | 5: 86803983 |
| 26168 | 5: 86807041 | 26195 | 5: 87419158 | 26197 | 5: 87436653 | 26205 | 5: 87558801 | 26222 | 5: 87935293 |
| 26227 | 5: 88025590 | 26232 | 5: 88169363 | 26240 | 5: 88321354 | 26241 | 5: 88337164 | 26246 | 5: 88393821 |
| 26251 | 5: 88442816 | 26257 | 5: 88527567 | 26259 | 5: 88560515 | 26260 | 5: 88562589 | 26264 | 5: 88656117 |
| 26265 | 5: 88656655 | 26266 | 5: 88663005 | 26267 | 5: 88664137 | 26268 | 5: 88664970 | 26269 | 5: 88665685 |
| 26270 | 5: 88667764 | 26271 | 5: 88668736 | 26272 | 5: 88669208 | 26273 | 5: 88670166 | 26274 | 5: 88670949 |
| 26275 | 5: 88671581 | 26276 | 5: 88672838 | 26277 | 5: 88673518 | 26278 | 5: 88675186 | 26279 | 5: 88676434 |
| 26280 | 5: 88677664 | 26281 | 5: 88679714 | 26282 | 5: 88681101 | 26289 | 5: 88791076 | 26294 | 5: 88871429 |
| 26295 | 5: 88903163 | 26297 | 5: 88927675 | 26303 | 5: 89099225 | 26311 | 5: 89264046 | 26313 | 5: 89283625 |
| 26315 | 5: 89343653 | 26316 | 5: 89359278 | 26317 | 5: 89364312 | 26327 | 5: 89668965 | 26337 | 5: 89886383 |
| 26338 | 5: 89886385 | 26346 | 5: 90068114 | 26353 | 5: 90321950 | 26354 | 5: 90345643 | 26356 | 5: 90409538 |
| 26363 | 5: 90604091 | 26369 | 5: 90678336 | 26375 | 5: 90778808 | 26425 | 5: 92149419 | 26439 | 5: 92379543 |
| 26479 | 5: 92796508 | 26480 | 5: 92796509 | 26481 | 5: 92796641 | 26482 | 5: 92796716 | 26486 | 5: 92864262 |
| 26490 | 5: 92953627 | 26493 | 5: 93100450 | 26512 | 5: 93520661 | 26515 | 5: 93603557 | 26517 | 5: 93614794 |
| 26539 | 5: 94115151 | 26543 | 5: 94190519 | 26550 | 5: 94345725 | 26551 | 5: 94346916 | 26555 | 5: 94480126 |
| 26560 | 5: 94697047 | 26567 | 5: 94796315 | 26568 | 5: 94806990 | 26586 | 5: 95221444 | 26588 | 5: 95236526 |
| 26590 | 5: 95266607 | 26592 | 5: 95275712 | 26609 | 5: 95732385 | 26613 | 5: 95783150 | 26623 | 5: 96041063 |
| 26630 | 5: 96115730 | 26634 | 5: 96229534 | 26636 | 5: 96266124 | 26641 | 5: 96344421 | 26648 | 5: 96479304 |
| 26650 | 5: 96555471 | 26653 | 5: 96570693 | 26654 | 5: 96587114 | 26657 | 5: 96683112 | 26658 | 5: 96697638 |
| 26659 | 5: 96722002 | 26663 | 5: 96826963 | 26665 | 5: 96891834 | 26666 | 5: 96895092 | 26667 | 5: 96897342 |
| 26668 | 5: 96903857 | 26669 | 5: 96904695 | 26670 | 5: 96905308 | 26672 | 5: 96908444 | 26673 | 5: 96909315 |
| 26678 | 5: 96986050 | 26682 | 5: 97026543 | 26686 | 5: 97080334 | 26696 | 5: 97322489 | 26701 | 5: 97423919 |
| 26702 | 5: 97434109 | 26704 | 5: 97466504 | 26705 | 5: 97487627 | 26710 | 5: 97624543 | 26727 | 5: 98048878 |
| 26731 | 5: 98072135 | 26741 | 5: 98284200 | 26743 | 5: 98321751 | 26746 | 5: 98348925 | 26747 | 5: 98360313 |
| 26748 | 5: 98369761 | 26753 | 5: 98558774 | 26754 | 5: 98558789 | 26755 | 5: 98558837 | 26756 | 5: 98558879 |
| 26757 | 5: 98559154 | 26758 | 5: 98559190 | 26759 | 5: 98559458 | 26760 | 5: 98559459 | 26762 | 5: 98571338 |
| 26769 | 5: 98750219 | 26771 | 5: 98819283 | 26774 | 5: 98902747 | 26779 | 5: 98952191 | 26782 | 5: 98988266 |
| 26783 | 5: 98991352 | 26786 | 5: 99023106 | 26793 | 5: 99430794 | 26800 | 5: 99597967 | 26801 | 5: 99613789 |
| 26803 | 5: 99657462 | 26804 | 5: 99657533 | 26805 | 5: 99657651 | 26806 | 5: 99657668 | 26808 | 5: 99674708 |
| 26809 | 5: 99678222 | 26811 | 5: 99698551 | 26812 | 5: 99698552 | 26815 | 5: 99764163 | 26817 | 5: 99773482 |
| 26819 | 5: 99784430 | 26827 | 5: 99916927 | 26837 | 5: 100075903 | 26838 | 5: 100085604 | 26839 | 5: 100089578 |
| 26841 | 5: 100109898 | 26842 | 5: 100126206 | 26843 | 5: 100133218 | 26846 | 5: 100234492 | 26848 | 5: 100300261 |
| 26849 | 5: 100311514 | 26850 | 5: 100341741 | 26852 | 5: 100377385 | 26854 | 5: 100381117 | 26856 | 5: 100389557 |
| 26858 | 5: 100426926 | 26866 | 5: 100696501 | 26867 | 5: 100696607 | 26868 | 5: 100698121 | 26869 | 5: 100698579 |
| 26872 | 5: 100728733 | 26881 | 5: 100884953 | 26901 | 5: 101256059 | 26903 | 5: 101355705 | 26904 | 5: 101371243 |
| 26906 | 5: 101513339 | 26911 | 5: 101595246 | 26915 | 5: 101614862 | 26916 | 5: 101616220 | 26927 | 5: 101872342 |
| 26929 | 5: 101932704 | 26930 | 5: 101932771 | 26942 | 5: 102469703 | 26956 | 5: 103101288 | 26957 | 5: 103104040 |
| 26959 | 5: 103119311 | 26960 | 5: 103119357 | 26961 | 5: 103119383 | 26962 | 5: 103119408 | 26963 | 5: 103119413 |
| 26965 | 5: 103127016 | 26969 | 5: 103180670 | 26970 | 5: 103181148 | 26971 | 5: 103186080 | 26972 | 5: 103186163 |
| 26975 | 5: 103229995 | 26976 | 5: 103292486 | 26977 | 5: 103293544 | 26979 | 5: 103324283 | 26987 | 5: 103577479 |
| 26992 | 5: 103749650 | 26993 | 5: 103753540 | 26994 | 5: 103765906 | 27009 | 5: 104024017 | 27010 | 5: 104047698 |
| 27011 | 5: 104049926 | 27012 | 5: 104051208 | 27014 | 5: 104058009 | 27015 | 5: 104067878 | 27016 | 5: 104090665 |
| 27019 | 5: 104166543 | 27022 | 5: 104306581 | 27027 | 5: 104414866 | 27028 | 5: 104421461 | 27029 | 5: 104425156 |
| 27030 | 5: 104425652 | 27035 | 5: 104475838 | 27038 | 5: 104487779 | 27048 | 5: 104725697 | 27052 | 5: 104812593 |
| 27057 | 5: 104944974 | 27059 | 5: 104962040 | 27065 | 5: 105070263 | 27066 | 5: 105095351 | 27070 | 5: 105138348 |
| 27071 | 5: 105144347 | 27088 | 5: 105596087 | 27097 | 5: 105756724 | 27103 | 5: 105773809 | 27107 | 5: 105778777 |
| 27108 | 5: 105779368 | 27151 | 5: 106320490 | 27154 | 5: 106351560 | 27160 | 5: 106474477 | 27171 | 5: 106583604 |
| 27173 | 5: 106588115 | 27177 | 5: 106701615 | 27184 | 5: 106816406 | 27190 | 5: 106904200 | 27191 | 5: 106904337 |
| 27192 | 5: 106904427 | 27193 | 5: 106904456 | 27194 | 5: 106906152 | 27196 | 5: 106921443 | 27198 | 5: 106952814 |
| 27199 | 5: 106952815 | 27208 | 5: 107090339 | 27210 | 5: 107152886 | 27211 | 5: 107155008 | 27213 | 5: 107195685 |
| 27214 | 5: 107203743 | 27226 | 5: 107421295 | 27230 | 5: 107445396 | 27236 | 5: 107578561 | 27239 | 5: 107683951 |
| 27242 | 5: 107725814 | 27258 | 5: 108050852 | 27260 | 5: 108055831 | 27261 | 5: 108055990 | 27264 | 5: 108099551 |
| 27266 | 5: 108207411 | 27267 | 5: 108207896 | 27270 | 5: 108230364 | 27272 | 5: 108235905 | 27277 | 5: 108323867 |
| 27296 | 5: 108547597 | 27312 | 5: 108942683 | 27314 | 5: 108955049 | 27316 | 5: 108984906 | 27320 | 5: 109034391 |
| 27322 | 5: 109075632 | 27323 | 5: 109090548 | 27324 | 5: 109096526 | 27325 | 5: 109153205 | 27327 | 5: 109154168 |
| 27328 | 5: 109178144 | 27329 | 5: 109178358 | 27330 | 5: 109179308 | 27335 | 5: 109320605 | 27339 | 5: 109412747 |
| 27348 | 5: 109611685 | 27349 | 5: 109615284 | 27352 | 5: 109687895 | 27354 | 5: 109722487 | 27356 | 5: 109749503 |
| 27357 | 5: 109754096 | 27361 | 5: 109808607 | 27362 | 5: 109847158 | 27368 | 5: 109988776 | 27369 | 5: 109989550 |
| 27371 | 5: 110069248 | 27375 | 5: 110112327 | 27379 | 5: 110193224 | 27383 | 5: 110314027 | 27384 | 5: 110353226 |
| 27385 | 5: 110369778 | 27386 | 5: 110373456 | 27387 | 5: 110373555 | 27389 | 5: 110374859 | 27393 | 5: 110398433 |
| 27397 | 5: 110509525 | 27400 | 5: 110531320 | 27401 | 5: 110531367 | 27402 | 5: 110532648 | 27403 | 5: 110532703 |
| 27404 | 5: 110532709 | 27405 | 5: 110533852 | 27406 | 5: 110533859 | 27407 | 5: 110534455 | 27410 | 5: 110564870 |
| 27412 | 5: 110569021 | 27413 | 5: 110569141 | 27414 | 5: 110569146 | 27419 | 5: 110751512 | 27431 | 5: 111053132 |
| 27433 | 5: 111063936 | 27434 | 5: 111065855 | 27435 | 5: 111065875 | 27446 | 5: 111321416 | 27452 | 5: 111501396 |
| 27453 | 5: 111502809 | 27460 | 5: 111753901 | 27465 | 5: 111845063 | 27468 | 5: 111862953 | 27469 | 5: 111864267 |
| 27480 | 5: 112115889 | 27481 | 5: 112115946 | 27482 | 5: 112126293 | 27483 | 5: 112126298 | 27484 | 5: 112133653 |
| 27485 | 5: 112135929 | 27489 | 5: 112235754 | 27492 | 5: 112343380 | 27495 | 5: 112408202 | 27498 | 5: 112448429 |
| 27499 | 5: 112455682 | 27505 | 5: 112604562 | 27507 | 5: 112634025 | 27508 | 5: 112641994 | 27510 | 5: 112722034 |
| 27513 | 5: 112805426 | 27516 | 5: 112917350 | 27527 | 5: 113148146 | 27528 | 5: 113148464 | 27529 | 5: 113149795 |
| 27550 | 5: 113769146 | 27556 | 5: 113920144 | 27558 | 5: 113928017 | 27564 | 5: 114059697 | 27573 | 5: 114343422 |
| 27574 | 5: 114343454 | 27577 | 5: 114569421 | 27581 | 5: 114723102 | 27590 | 5: 115002143 | 27593 | 5: 115052949 |
| 27600 | 5: 115255504 | 27601 | 5: 115265500 | 27608 | 5: 115466163 | 27615 | 5: 115514965 | 27616 | 5: 115517449 |
| 27626 | 5: 115682503 | 27629 | 5: 115766557 | 27651 | 5: 116121476 | 27661 | 5: 116390999 | 27663 | 5: 116423647 |
| 27665 | 5: 116464508 | 27669 | 5: 116511553 | 27672 | 5: 116537986 | 27674 | 5: 116577132 | 27685 | 5: 116593122 |
| 27686 | 5: 116593191 | 27687 | 5: 116593192 | 27689 | 5: 116593706 | 27690 | 5: 116593715 | 27691 | 5: 116593762 |
| 27692 | 5: 116593794 | 27693 | 5: 116593850 | 27703 | 5: 116596428 | 27704 | 5: 116596441 | 27705 | 5: 116596625 |
| 27709 | 5: 116597945 | 27710 | 5: 116598050 | 27713 | 5: 116600211 | 27714 | 5: 116601776 | 27717 | 5: 116622560 |
| 27723 | 5: 116699001 | 27724 | 5: 116699079 | 27725 | 5: 116699138 | 27726 | 5: 116699145 | 27727 | 5: 116699159 |
| 27732 | 5: 116743643 | 27734 | 5: 116745570 | 27735 | 5: 116757705 | 27740 | 5: 116908657 | 27741 | 5: 116911216 |
| 27742 | 5: 116911371 | 27743 | 5: 116911376 | 27744 | 5: 116915304 | 27745 | 5: 116915313 | 27746 | 5: 116915402 |
| 27748 | 5: 116922121 | 27751 | 5: 116973879 | 27753 | 5: 116989243 | 27754 | 5: 116991259 | 27762 | 5: 117097690 |
| 27767 | 5: 117234102 | 27769 | 5: 117262549 | 27770 | 5: 117262555 | 27771 | 5: 117262556 | 27773 | 5: 117262630 |
| 27785 | 5: 117459589 | 27786 | 5: 117469696 | 27787 | 5: 117471979 | 27788 | 5: 117473591 | 27789 | 5: 117474224 |
| 27790 | 5: 117475215 | 27791 | 5: 117476538 | 27799 | 5: 117579793 | 27805 | 5: 117645005 | 27815 | 5: 117785747 |
| 27818 | 5: 117850396 | 27844 | 5: 118401624 | 27854 | 5: 118620070 | 27859 | 5: 118696815 | 27875 | 5: 118913308 |
| 27879 | 5: 118954058 | 27880 | 5: 118959454 | 27881 | 5: 118971379 | 27882 | 5: 118972496 | 27883 | 5: 118978705 |
| 27885 | 5: 118982714 | 27900 | 5: 119181604 | 27915 | 5: 119570841 | 27918 | 5: 119667915 | 27921 | 5: 119771063 |
| 27922 | 5: 119774093 | 27923 | 5: 119775410 | 27926 | 5: 119853084 | 27931 | 5: 119945721 | 27932 | 5: 119995446 |
| 27964 | 6: 844150 | 27965 | 6: 844153 | 27972 | 6: 1031446 | 27980 | 6: 1285962 | 27990 | 6: 1702526 |
| 27992 | 6: 1761466 | 27993 | 6: 1761542 | 27994 | 6: 1761563 | 27995 | 6: 1774727 | 27998 | 6: 1945674 |
| 28030 | 6: 2057409 | 28044 | 6: 2394123 | 28046 | 6: 2394185 | 28047 | 6: 2418816 | 28048 | 6: 2418834 |
| 28054 | 6: 2624789 | 28055 | 6: 2625544 | 28058 | 6: 2663078 | 28059 | 6: 2672214 | 28077 | 6: 3108815 |
| 28078 | 6: 3108854 | 28087 | 6: 3376371 | 28088 | 6: 3380776 | 28089 | 6: 3383123 | 28090 | 6: 3386603 |
| 28091 | 6: 3388251 | 28092 | 6: 3392342 | 28094 | 6: 3398350 | 28095 | 6: 3399218 | 28096 | 6: 3399858 |
| 28097 | 6: 3402165 | 28099 | 6: 3423360 | 28100 | 6: 3425355 | 28102 | 6: 3444538 | 28104 | 6: 3449326 |
| 28106 | 6: 3469199 | 28110 | 6: 3575704 | 28111 | 6: 3585145 | 28112 | 6: 3587923 | 28113 | 6: 3587933 |
| 28114 | 6: 3588243 | 28115 | 6: 3588261 | 28116 | 6: 3589475 | 28117 | 6: 3589994 | 28124 | 6: 3680340 |
| 28142 | 6: 4189994 | 28143 | 6: 4197131 | 28147 | 6: 4297910 | 28149 | 6: 4300166 | 28179 | 6: 4998395 |
| 28183 | 6: 5135323 | 28188 | 6: 5332029 | 28190 | 6: 5359913 | 28191 | 6: 5363670 | 28192 | 6: 5366571 |
| 28193 | 6: 5417516 | 28194 | 6: 5420554 | 28198 | 6: 5638260 | 28208 | 6: 5736468 | 28213 | 6: 5970478 |
| 28215 | 6: 6023720 | 28217 | 6: 6068366 | 28218 | 6: 6086349 | 28219 | 6: 6090367 | 28220 | 6: 6099226 |
| 28222 | 6: 6135400 | 28223 | 6: 6135571 | 28224 | 6: 6135977 | 28225 | 6: 6142753 | 28226 | 6: 6145385 |
| 28227 | 6: 6146291 | 28229 | 6: 6150122 | 28231 | 6: 6213823 | 28233 | 6: 6252521 | 28236 | 6: 6303620 |
| 28237 | 6: 6304251 | 28241 | 6: 6327370 | 28246 | 6: 6434692 | 28248 | 6: 6445175 | 28251 | 6: 6505947 |
| 28252 | 6: 6508802 | 28253 | 6: 6508845 | 28256 | 6: 6598639 | 28257 | 6: 6630222 | 28259 | 6: 6653707 |
| 28260 | 6: 6653733 | 28261 | 6: 6653820 | 28262 | 6: 6654166 | 28263 | 6: 6654264 | 28265 | 6: 6674156 |
| 28266 | 6: 6675058 | 28296 | 6: 7438091 | 28297 | 6: 7438117 | 28306 | 6: 7840060 | 28315 | 6: 8178961 |
| 28325 | 6: 8498664 | 28330 | 6: 8732227 | 28331 | 6: 8782400 | 28338 | 6: 8951172 | 28340 | 6: 8957062 |
| 28344 | 6: 8978291 | 28348 | 6: 8993893 | 28359 | 6: 9166519 | 28360 | 6: 9196740 | 28361 | 6: 9200024 |
| 28362 | 6: 9200854 | 28363 | 6: 9201774 | 28364 | 6: 9203527 | 28365 | 6: 9204459 | 28366 | 6: 9205411 |
| 28367 | 6: 9207235 | 28368 | 6: 9208280 | 28369 | 6: 9209206 | 28411 | 6: 10509217 | 28417 | 6: 10679784 |
| 28436 | 6: 11171281 | 28444 | 6: 11394489 | 28448 | 6: 11465975 | 28461 | 6: 11714410 | 28468 | 6: 11859669 |
| 28469 | 6: 11867154 | 28471 | 6: 11887100 | 28489 | 6: 12266091 | 28492 | 6: 12331408 | 28493 | 6: 12406590 |
| 28496 | 6: 12449553 | 28499 | 6: 12460758 | 28505 | 6: 12583026 | 28520 | 6: 12951400 | 28523 | 6: 13000409 |
| 28524 | 6: 13001855 | 28525 | 6: 13027415 | 28528 | 6: 13118294 | 28531 | 6: 13124831 | 28532 | 6: 13124859 |
| 28533 | 6: 13124885 | 28539 | 6: 13212064 | 28550 | 6: 13497569 | 28554 | 6: 13707108 | 28566 | 6: 14075733 |
| 28572 | 6: 14295399 | 28592 | 6: 14776571 | 28593 | 6: 14795242 | 28596 | 6: 14878264 | 28598 | 6: 14937683 |
| 28614 | 6: 15271409 | 28615 | 6: 15279031 | 28628 | 6: 15511470 | 28631 | 6: 15552360 | 28632 | 6: 15552836 |
| 28633 | 6: 15552929 | 28634 | 6: 15554381 | 28635 | 6: 15554470 | 28639 | 6: 15665547 | 28640 | 6: 15665565 |
| 28644 | 6: 15724964 | 28659 | 6: 16024226 | 28672 | 6: 16407876 | 28675 | 6: 16429738 | 28676 | 6: 16431811 |
| 28680 | 6: 16480595 | 28681 | 6: 16483148 | 28688 | 6: 16631779 | 28690 | 6: 16706078 | 28692 | 6: 16770941 |
| 28701 | 6: 16924337 | 28702 | 6: 16926868 | 28704 | 6: 16957756 | 28710 | 6: 17056311 | 28711 | 6: 17056315 |
| 28712 | 6: 17080889 | 28734 | 6: 17680046 | 28737 | 6: 17695292 | 28746 | 6: 17927268 | 28747 | 6: 17931957 |
| 28748 | 6: 17938149 | 28761 | 6: 18039717 | 28772 | 6: 18212765 | 28777 | 6: 18284502 | 28778 | 6: 18314365 |
| 28789 | 6: 18646895 | 28790 | 6: 18701849 | 28791 | 6: 18731749 | 28799 | 6: 18864835 | 28807 | 6: 19042586 |
| 28809 | 6: 19074411 | 28811 | 6: 19074485 | 28815 | 6: 19190465 | 28826 | 6: 19438867 | 28827 | 6: 19439848 |
| 28829 | 6: 19456967 | 28830 | 6: 19462913 | 28831 | 6: 19495081 | 28839 | 6: 19649377 | 28848 | 6: 19872402 |
| 28850 | 6: 19894311 | 28851 | 6: 19923403 | 28858 | 6: 20135992 | 28860 | 6: 20184822 | 28861 | 6: 20192872 |
| 28862 | 6: 20206522 | 28872 | 6: 20397821 | 28876 | 6: 20495916 | 28881 | 6: 20600966 | 28882 | 6: 20600990 |
| 28907 | 6: 21325769 | 28908 | 6: 21355207 | 28910 | 6: 21392641 | 28913 | 6: 21439632 | 28914 | 6: 21439691 |
| 28915 | 6: 21439763 | 28921 | 6: 21659954 | 28922 | 6: 21672266 | 28923 | 6: 21678873 | 28925 | 6: 21686069 |
| 28926 | 6: 21691325 | 28927 | 6: 21703290 | 28928 | 6: 21714263 | 28933 | 6: 21771563 | 28934 | 6: 21772453 |
| 28935 | 6: 21777544 | 28939 | 6: 21951701 | 28942 | 6: 22033622 | 28962 | 6: 22379732 | 28964 | 6: 22422233 |
| 28967 | 6: 22500214 | 28968 | 6: 22500233 | 28991 | 6: 23037086 | 28997 | 6: 23243928 | 29000 | 6: 23295269 |
| 29005 | 6: 23444102 | 29006 | 6: 23457488 | 29010 | 6: 23533175 | 29013 | 6: 23555860 | 29015 | 6: 23571729 |
| 29024 | 6: 23873913 | 29029 | 6: 23921917 | 29047 | 6: 24373068 | 29051 | 6: 24503230 | 29053 | 6: 24518463 |
| 29064 | 6: 24837558 | 29070 | 6: 24942515 | 29071 | 6: 24948898 | 29076 | 6: 25007714 | 29077 | 6: 25024841 |
| 29078 | 6: 25039222 | 29083 | 6: 25164226 | 29095 | 6: 25528726 | 29100 | 6: 25695588 | 29102 | 6: 25788477 |
| 29108 | 6: 25887098 | 29117 | 6: 26022919 | 29118 | 6: 26033719 | 29127 | 6: 26200013 | 29129 | 6: 26282778 |
| 29130 | 6: 26290561 | 29139 | 6: 26655531 | 29140 | 6: 26655687 | 29142 | 6: 26679658 | 29163 | 6: 27215526 |
| 29167 | 6: 27355784 | 29174 | 6: 27472557 | 29186 | 6: 27864310 | 29212 | 6: 28647979 | 29231 | 6: 29128064 |
| 29237 | 6: 29212166 | 29239 | 6: 29328156 | 29240 | 6: 29328334 | 29241 | 6: 29328350 | 29243 | 6: 29376641 |
| 29245 | 6: 29389036 | 29247 | 6: 29435290 | 29253 | 6: 29483476 | 29259 | 6: 29565346 | 29265 | 6: 29684500 |
| 29279 | 6: 30075635 | 29342 | 6: 30145040 | 29365 | 6: 30563336 | 29399 | 6: 31576983 | 29412 | 6: 31922816 |
| 29421 | 6: 32195477 | 29425 | 6: 32453198 | 29426 | 6: 32493429 | 29468 | 6: 34036502 | 29472 | 6: 34067879 |
| 29473 | 6: 34068521 | 29486 | 6: 34217305 | 29587 | 6: 34344002 | 29660 | 6: 34467878 | 29733 | 6: 34605510 |
| 29954 | 6: 35510819 | 29957 | 6: 35561016 | 29986 | 6: 35857940 | 30022 | 6: 36053886 | 30024 | 6: 36074911 |
| 30130 | 6: 36395104 | 30139 | 6: 36431315 | 30145 | 6: 36444505 | 30148 | 6: 36448732 | 30174 | 6: 36489959 |
| 30250 | 6: 36553066 | 30255 | 6: 36561452 | 30300 | 6: 36642083 | 30317 | 6: 36688082 | 30321 | 6: 36706370 |
| 30383 | 6: 36857123 | 30808 | 6: 37218423 | 31143 | 6: 37396789 | 31963 | 6: 39186781 | 31964 | 6: 39249570 |
| 31972 | 6: 39371669 | 31973 | 6: 39392848 | 31997 | 6: 39755061 | 32033 | 6: 40227846 | 32038 | 6: 40262465 |
| 32095 | 6: 40813933 | 32099 | 6: 40846711 | 32101 | 6: 40869544 | 32162 | 6: 41204988 | 32163 | 6: 41205018 |
| 32194 | 6: 41998863 | 32195 | 6: 42004652 | 32196 | 6: 42005216 | 32197 | 6: 42010829 | 32198 | 6: 42011964 |
| 32199 | 6: 42014380 | 32200 | 6: 42017579 | 32202 | 6: 42030911 | 32206 | 6: 42098249 | 32238 | 6: 42816146 |
| 32247 | 6: 42950720 | 32272 | 6: 43082880 | 32273 | 6: 43090582 | 32274 | 6: 43121398 | 32288 | 6: 43377340 |
| 32289 | 6: 43378488 | 32310 | 6: 43688837 | 32324 | 6: 43887416 | 32334 | 6: 44040805 | 32335 | 6: 44059941 |
| 32336 | 6: 44072335 | 32349 | 6: 44361206 | 32359 | 6: 44628166 | 32367 | 6: 44692016 | 32483 | 6: 45118837 |
| 32493 | 6: 45218126 | 32498 | 6: 45277318 | 32501 | 6: 45322457 | 32503 | 6: 45335602 | 32504 | 6: 45364598 |
| 32507 | 6: 45390455 | 32509 | 6: 45416952 | 32513 | 6: 45505416 | 32514 | 6: 45522021 | 32520 | 6: 45688164 |
| 32531 | 6: 45935668 | 32532 | 6: 45982939 | 32534 | 6: 46022376 | 32535 | 6: 46078755 | 32545 | 6: 46209779 |
| 32550 | 6: 46337526 | 32552 | 6: 46378480 | 32553 | 6: 46389387 | 32554 | 6: 46390042 | 32577 | 6: 46954860 |
| 32582 | 6: 47076276 | 32595 | 6: 47353083 | 32606 | 6: 47640345 | 32616 | 6: 47866852 | 32618 | 6: 47903812 |
| 32622 | 6: 48018241 | 32634 | 6: 48398260 | 32642 | 6: 48750204 | 32664 | 6: 49330544 | 32666 | 6: 49366546 |
| 32681 | 6: 49731100 | 32682 | 6: 49732769 | 32683 | 6: 49734879 | 32684 | 6: 49738480 | 32685 | 6: 49759413 |
| 32686 | 6: 49766766 | 32687 | 6: 49773026 | 32690 | 6: 49804879 | 32691 | 6: 49816719 | 32697 | 6: 49895642 |
| 32705 | 6: 50190150 | 32709 | 6: 50252554 | 32711 | 6: 50316194 | 32712 | 6: 50316384 | 32713 | 6: 50316707 |
| 32714 | 6: 50342873 | 32745 | 6: 51287866 | 32761 | 6: 51779722 | 32770 | 6: 52097852 | 32771 | 6: 52097941 |
| 32776 | 6: 52230394 | 32777 | 6: 52230455 | 32779 | 6: 52258121 | 32811 | 6: 52492036 | 32823 | 6: 52776675 |
| 32839 | 6: 53189333 | 32841 | 6: 53215914 | 32845 | 6: 53260563 | 32856 | 6: 53542273 | 32858 | 6: 53587223 |
| 32871 | 6: 53982975 | 32882 | 6: 54231133 | 32895 | 6: 54705767 | 32905 | 6: 55072307 | 32957 | 6: 55697219 |
| 32965 | 6: 55964167 | 32981 | 6: 56569534 | 32982 | 6: 56580948 | 32997 | 6: 56927864 | 32998 | 6: 56927881 |
| 32999 | 6: 56927920 | 33000 | 6: 56928100 | 33011 | 6: 57258298 | 33013 | 6: 57310043 | 33016 | 6: 57383139 |
| 33019 | 6: 57452741 | 33021 | 6: 57538068 | 33024 | 6: 57589915 | 33053 | 6: 58398195 | 33055 | 6: 58507178 |
| 33065 | 6: 58716078 | 33075 | 6: 59092959 | 33080 | 6: 59188156 | 33085 | 6: 59262762 | 33100 | 6: 59749739 |
| 33108 | 6: 59853909 | 33118 | 6: 59966318 | 33123 | 6: 60097949 | 33128 | 6: 60226786 | 33130 | 6: 60265173 |
| 33131 | 6: 60301627 | 33133 | 6: 60345603 | 33135 | 6: 60373758 | 33138 | 6: 60422003 | 33140 | 6: 60468668 |
| 33152 | 6: 60773644 | 33166 | 6: 61158905 | 33167 | 6: 61161272 | 33172 | 6: 61280689 | 33177 | 6: 61397201 |
| 33179 | 6: 61410508 | 33180 | 6: 61421440 | 33187 | 6: 61548278 | 33188 | 6: 61582228 | 33189 | 6: 61589076 |
| 33190 | 6: 61673425 | 33217 | 6: 62603953 | 33226 | 6: 62889998 | 33231 | 6: 62988117 | 33232 | 6: 63010364 |
| 33233 | 6: 63010380 | 33235 | 6: 63029593 | 33237 | 6: 63097017 | 33247 | 6: 63307538 | 33248 | 6: 63308671 |
| 33249 | 6: 63347756 | 33269 | 6: 64151594 | 33281 | 6: 64662329 | 33282 | 6: 64665326 | 33293 | 6: 65016085 |
| 33300 | 6: 65345032 | 33302 | 6: 65383049 | 33304 | 6: 65420404 | 33305 | 6: 65422497 | 33306 | 6: 65422592 |
| 33307 | 6: 65456807 | 33325 | 6: 66109765 | 33332 | 6: 66343250 | 33334 | 6: 66387388 | 33340 | 6: 66651619 |
| 33345 | 6: 66831939 | 33348 | 6: 66876573 | 33353 | 6: 66988792 | 33356 | 6: 67035579 | 33358 | 6: 67089021 |
| 33373 | 6: 67582129 | 33382 | 6: 68017233 | 33387 | 6: 68177405 | 33390 | 6: 68268998 | 33395 | 6: 68352179 |
| 33401 | 6: 68565298 | 33402 | 6: 68589087 | 33403 | 6: 68598341 | 33410 | 6: 68728215 | 33413 | 6: 68763773 |
| 33415 | 6: 68828972 | 33417 | 6: 68885563 | 33420 | 6: 68935879 | 33426 | 6: 69138647 | 33428 | 6: 69168309 |
| 33430 | 6: 69216815 | 33434 | 6: 69287580 | 33436 | 6: 69352586 | 33439 | 6: 69439499 | 33445 | 6: 69755083 |
| 33452 | 6: 69895326 | 33464 | 6: 70383634 | 33465 | 6: 70399966 | 33466 | 6: 70404486 | 33467 | 6: 70405959 |
| 33468 | 6: 70423245 | 33472 | 6: 70564450 | 33481 | 6: 70877482 | 33482 | 6: 70900974 | 33490 | 6: 71126939 |
| 33494 | 6: 71294876 | 33504 | 6: 71562156 | 33510 | 6: 71703879 | 33518 | 6: 72060315 | 33522 | 6: 72174384 |
| 33531 | 6: 72351300 | 33533 | 6: 72379846 | 33540 | 6: 72542846 | 33548 | 6: 72783452 | 33558 | 6: 73223485 |
| 33565 | 6: 73414475 | 33567 | 6: 73481332 | 33570 | 6: 73620191 | 33577 | 6: 73801067 | 33631 | 6: 75798192 |
| 33645 | 6: 76545846 | 33646 | 6: 76546983 | 33647 | 6: 76547862 | 33665 | 6: 76855629 | 33677 | 6: 77240842 |
| 33680 | 6: 77349471 | 33691 | 6: 77688509 | 33694 | 6: 77787079 | 33711 | 6: 78425706 | 33712 | 6: 78429257 |
| 33717 | 6: 78761621 | 33718 | 6: 78761714 | 33721 | 6: 78917750 | 33743 | 6: 79765417 | 33765 | 6: 80471442 |
| 33766 | 6: 80482749 | 33767 | 6: 80484712 | 33768 | 6: 80491703 | 33769 | 6: 80530130 | 33770 | 6: 80567928 |
| 33771 | 6: 80590178 | 33773 | 6: 80675168 | 33788 | 6: 81051057 | 33814 | 6: 82117911 | 33855 | 6: 83277672 |
| 33858 | 6: 83308141 | 33861 | 6: 83347263 | 33864 | 6: 83451575 | 33865 | 6: 83451628 | 33866 | 6: 83451712 |
| 33867 | 6: 83451781 | 33868 | 6: 83451862 | 33870 | 6: 83469477 | 33871 | 6: 83470850 | 33872 | 6: 83475021 |
| 33884 | 6: 83758356 | 33885 | 6: 83763398 | 33892 | 6: 83946894 | 33893 | 6: 83948279 | 33894 | 6: 83950501 |
| 33898 | 6: 83985933 | 33902 | 6: 84087846 | 33903 | 6: 84138032 | 33910 | 6: 84497175 | 33911 | 6: 84497631 |
| 33912 | 6: 84497709 | 33913 | 6: 84511587 | 33914 | 6: 84511656 | 33915 | 6: 84513009 | 33932 | 6: 84975132 |
| 33933 | 6: 85067620 | 33934 | 6: 85092477 | 33935 | 6: 85092499 | 33936 | 6: 85092523 | 33937 | 6: 85093071 |
| 33938 | 6: 85108153 | 33939 | 6: 85108165 | 33940 | 6: 85108432 | 33941 | 6: 85108773 | 33943 | 6: 85136420 |
| 33944 | 6: 85137180 | 33950 | 6: 85315912 | 33977 | 6: 85896040 | 33978 | 6: 85896097 | 33979 | 6: 85896105 |
| 33980 | 6: 85896139 | 33981 | 6: 85906390 | 33982 | 6: 85908497 | 34003 | 6: 86508590 | 34012 | 6: 86710155 |
| 34028 | 6: 86901499 | 34032 | 6: 86989414 | 34034 | 6: 87007077 | 34040 | 6: 87076603 | 34043 | 6: 87111828 |
| 34057 | 6: 87463348 | 34067 | 6: 87603473 | 34074 | 6: 87731662 | 34082 | 6: 87796536 | 34085 | 6: 87923433 |
| 34086 | 6: 87923536 | 34092 | 6: 88075544 | 34096 | 6: 88164435 | 34097 | 6: 88184411 | 34098 | 6: 88184633 |
| 34102 | 6: 88318314 | 34108 | 6: 88486850 | 34116 | 6: 88859149 | 34130 | 6: 89152164 | 34131 | 6: 89181036 |
| 34150 | 6: 89663234 | 34152 | 6: 89663319 | 34153 | 6: 89663348 | 34154 | 6: 89674618 | 34162 | 6: 89768711 |
| 34170 | 6: 89975431 | 34182 | 6: 90390426 | 34185 | 6: 90416932 | 34186 | 6: 90457703 | 34196 | 6: 90680946 |
| 34199 | 6: 90714640 | 34200 | 6: 90726127 | 34206 | 6: 90794035 | 34208 | 6: 90816980 | 34210 | 6: 90823124 |
| 34211 | 6: 90826546 | 34212 | 6: 90826598 | 34213 | 6: 90827159 | 34214 | 6: 90836066 | 34215 | 6: 90836072 |
| 34216 | 6: 90836166 | 34221 | 6: 90946704 | 34228 | 6: 91105837 | 34229 | 6: 91122456 | 34236 | 6: 91204532 |
| 34237 | 6: 91223593 | 34238 | 6: 91226875 | 34239 | 6: 91246239 | 34240 | 6: 91275706 | 34241 | 6: 91282834 |
| 34242 | 6: 91284101 | 34243 | 6: 91284414 | 34244 | 6: 91285011 | 34245 | 6: 91285694 | 34246 | 6: 91285851 |
| 34247 | 6: 91285925 | 34248 | 6: 91290335 | 34249 | 6: 91294383 | 34250 | 6: 91322998 | 34257 | 6: 91406353 |
| 34258 | 6: 91408596 | 34260 | 6: 91433962 | 34261 | 6: 91462732 | 34271 | 6: 91615288 | 34272 | 6: 91616087 |
| 34275 | 6: 91649638 | 34277 | 6: 91692231 | 34280 | 6: 91736404 | 34282 | 6: 91751612 | 34284 | 6: 91836628 |
| 34287 | 6: 91878223 | 34291 | 6: 91994872 | 34300 | 6: 92336028 | 34302 | 6: 92383295 | 34308 | 6: 92595572 |
| 34310 | 6: 92678382 | 34313 | 6: 92774897 | 34317 | 6: 92832688 | 34335 | 6: 93291707 | 34336 | 6: 93291712 |
| 34337 | 6: 93291776 | 34343 | 6: 93354640 | 34344 | 6: 93363500 | 34351 | 6: 93496619 | 34353 | 6: 93533733 |
| 34361 | 6: 93646233 | 34373 | 6: 93947051 | 34378 | 6: 94069815 | 34389 | 6: 94150648 | 34392 | 6: 94188032 |
| 34393 | 6: 94201168 | 34394 | 6: 94204543 | 34395 | 6: 94215369 | 34401 | 6: 94394468 | 34403 | 6: 94416755 |
| 34413 | 6: 94854202 | 34424 | 6: 95339487 | 34426 | 6: 95406474 | 34427 | 6: 95406527 | 34429 | 6: 95406556 |
| 34432 | 6: 95553456 | 34441 | 6: 95787499 | 34444 | 6: 95884565 | 34449 | 6: 96032153 | 34451 | 6: 96087038 |
| 34462 | 6: 96247005 | 34474 | 6: 96452752 | 34496 | 6: 97018222 | 34504 | 6: 97179175 | 34505 | 6: 97179176 |
| 34506 | 6: 97179179 | 34507 | 6: 97179203 | 34508 | 6: 97179214 | 34509 | 6: 97179248 | 34510 | 6: 97217522 |
| 34511 | 6: 97226901 | 34512 | 6: 97244565 | 34513 | 6: 97244652 | 34514 | 6: 97244662 | 34515 | 6: 97255320 |
| 34516 | 6: 97255455 | 34517 | 6: 97255461 | 34528 | 6: 97447881 | 34529 | 6: 97449063 | 34530 | 6: 97453689 |
| 34544 | 6: 97650137 | 34547 | 6: 97742989 | 34556 | 6: 98047323 | 34558 | 6: 98052606 | 34566 | 6: 98256336 |
| 34570 | 6: 98334890 | 34571 | 6: 98337762 | 34572 | 6: 98337909 | 34573 | 6: 98337919 | 34574 | 6: 98338489 |
| 34575 | 6: 98338596 | 34576 | 6: 98338640 | 34577 | 6: 98338644 | 34579 | 6: 98372826 | 34581 | 6: 98379884 |
| 34596 | 6: 98671599 | 34608 | 6: 98974175 | 34628 | 6: 99529167 | 34637 | 6: 99783201 | 34641 | 6: 99954538 |
| 34669 | 6: 100390632 | 34671 | 6: 100431779 | 34678 | 6: 100585876 | 34685 | 6: 100727406 | 34689 | 6: 100852199 |
| 34693 | 6: 100953221 | 34706 | 6: 101294567 | 34707 | 6: 101294597 | 34714 | 6: 101450871 | 34729 | 6: 101787815 |
| 34730 | 6: 101837470 | 34739 | 6: 101917014 | 34740 | 6: 101935968 | 34741 | 6: 101941367 | 34744 | 6: 101973857 |
| 34754 | 6: 102148393 | 34755 | 6: 102148443 | 34760 | 6: 102234263 | 34766 | 6: 102424852 | 34772 | 6: 102510343 |
| 34779 | 6: 102606444 | 34783 | 6: 102645168 | 34784 | 6: 102645192 | 34785 | 6: 102645198 | 34788 | 6: 102756289 |
| 34791 | 6: 102773226 | 34793 | 6: 102854233 | 34795 | 6: 102855258 | 34796 | 6: 102857313 | 34797 | 6: 102870507 |
| 34798 | 6: 102871524 | 34799 | 6: 102872128 | 34800 | 6: 102873595 | 34801 | 6: 102875781 | 34802 | 6: 102889726 |
| 34803 | 6: 102890450 | 34804 | 6: 102892630 | 34805 | 6: 102896127 | 34806 | 6: 102896985 | 34807 | 6: 102927406 |
| 34812 | 6: 103013214 | 34822 | 6: 103212156 | 34835 | 6: 103357734 | 34837 | 6: 103396597 | 34839 | 6: 103431771 |
| 34841 | 6: 103480885 | 34842 | 6: 103490310 | 34843 | 6: 103490339 | 34844 | 6: 103490470 | 34848 | 6: 103557818 |
| 34851 | 6: 103637428 | 34852 | 6: 103640406 | 34854 | 6: 103654616 | 34855 | 6: 103663732 | 34856 | 6: 103741666 |
| 34857 | 6: 103773464 | 34858 | 6: 103774096 | 34862 | 6: 103862052 | 34864 | 6: 103898267 | 34876 | 6: 104267666 |
| 34881 | 6: 104339504 | 34892 | 6: 104657713 | 34893 | 6: 104658492 | 34897 | 6: 104793010 | 34898 | 6: 104793414 |
| 34899 | 6: 104809492 | 34900 | 6: 104809647 | 34901 | 6: 104824268 | 34903 | 6: 104868243 | 34905 | 6: 104881408 |
| 34909 | 6: 104928796 | 34914 | 6: 105104413 | 34924 | 6: 105290637 | 34927 | 6: 105378109 | 34929 | 6: 105423924 |
| 34931 | 6: 105504062 | 34940 | 6: 105638984 | 34941 | 6: 105650631 | 34950 | 6: 105886896 | 34959 | 6: 106123065 |
| 34966 | 6: 106312210 | 34971 | 6: 106540140 | 34979 | 6: 106739419 | 34986 | 6: 106953368 | 35009 | 6: 107508591 |
| 35024 | 6: 108020924 | 35045 | 6: 108596491 | 35055 | 6: 108951951 | 35057 | 6: 108952102 | 35059 | 6: 108993006 |
| 35068 | 6: 109199682 | 35071 | 6: 109282471 | 35072 | 6: 109282486 | 35102 | 6: 109823696 | 35111 | 6: 110018683 |
| 35142 | 6: 110852081 | 35156 | 6: 111034643 | 35157 | 6: 111034669 | 35160 | 6: 111043273 | 35167 | 6: 111198616 |
| 35176 | 6: 111399485 | 35180 | 6: 111474948 | 35189 | 6: 111673792 | 35198 | 6: 111776075 | 35200 | 6: 111795733 |
| 35201 | 6: 111796366 | 35205 | 6: 111879814 | 35206 | 6: 111880060 | 35208 | 6: 111944736 | 35209 | 6: 111951572 |
| 35226 | 6: 112530579 | 35231 | 6: 112650810 | 35234 | 6: 112672985 | 35239 | 6: 112735477 | 35241 | 6: 112750366 |
| 35247 | 6: 112863762 | 35251 | 6: 112908010 | 35255 | 6: 112989544 | 35262 | 6: 113261157 | 35272 | 6: 113442820 |
| 35276 | 6: 113471367 | 35277 | 6: 113480954 | 35281 | 6: 113591450 | 35288 | 6: 113702935 | 35290 | 6: 113754589 |
| 35291 | 6: 113756287 | 35292 | 6: 113756982 | 35293 | 6: 113765934 | 35295 | 6: 113775951 | 35297 | 6: 113799562 |
| 35298 | 6: 113821699 | 35300 | 6: 113827124 | 35301 | 6: 113830450 | 35309 | 6: 113999468 | 35311 | 6: 114031327 |
| 35312 | 6: 114040384 | 35320 | 6: 114163874 | 35324 | 6: 114205337 | 35327 | 6: 114271753 | 35334 | 6: 114428994 |
| 35336 | 6: 114452241 | 35337 | 6: 114471056 | 35342 | 6: 114583005 | 35344 | 6: 114601159 | 35345 | 6: 114602046 |
| 35351 | 6: 114719008 | 35355 | 6: 114797024 | 35365 | 6: 115032289 | 35366 | 6: 115035590 | 35367 | 6: 115035592 |
| 35376 | 6: 115201677 | 35382 | 6: 115369888 | 35401 | 6: 115856852 | 35403 | 6: 115872479 | 35404 | 6: 115959886 |
| 35408 | 6: 116004094 | 35409 | 6: 116011104 | 35416 | 6: 116154808 | 35417 | 6: 116160046 | 35420 | 6: 116200574 |
| 35422 | 6: 116286341 | 35425 | 6: 116319165 | 35430 | 6: 116485262 | 35431 | 6: 116485283 | 35432 | 6: 116485335 |
| 35433 | 6: 116485360 | 35434 | 6: 116485372 | 35435 | 6: 116485909 | 35436 | 6: 116487900 | 35437 | 6: 116487922 |
| 35438 | 6: 116488922 | 35439 | 6: 116489686 | 35440 | 6: 116489801 | 35441 | 6: 116500010 | 35442 | 6: 116500063 |
| 35443 | 6: 116500364 | 35444 | 6: 116500379 | 35445 | 6: 116509870 | 35446 | 6: 116514508 | 35447 | 6: 116514529 |
| 35453 | 6: 116932235 | 35457 | 6: 117212852 | 35458 | 6: 117247186 | 35459 | 6: 117253860 | 35462 | 6: 117399949 |
| 35463 | 6: 117405411 | 35464 | 6: 117444202 | 35465 | 6: 117445801 | 35466 | 6: 117445935 | 35467 | 6: 117463206 |
| 35490 | 7: 516102 | 35502 | 7: 810744 | 35503 | 7: 822079 | 35533 | 7: 1434030 | 35534 | 7: 1439391 |
| 35535 | 7: 1444418 | 35537 | 7: 1508351 | 35539 | 7: 1552952 | 35546 | 7: 1663078 | 35547 | 7: 1663134 |
| 35550 | 7: 1706720 | 35553 | 7: 1757821 | 35560 | 7: 1977089 | 35562 | 7: 1996339 | 35563 | 7: 2036084 |
| 35567 | 7: 2112952 | 35577 | 7: 2352038 | 35587 | 7: 2461961 | 35590 | 7: 2556375 | 35594 | 7: 2628007 |
| 35595 | 7: 2628008 | 35596 | 7: 2628014 | 35597 | 7: 2628018 | 35598 | 7: 2628066 | 35599 | 7: 2628069 |
| 35600 | 7: 2628233 | 35601 | 7: 2628255 | 35602 | 7: 2628256 | 35604 | 7: 2630943 | 35605 | 7: 2630947 |
| 35606 | 7: 2631054 | 35607 | 7: 2631064 | 35610 | 7: 2682673 | 35611 | 7: 2682716 | 35612 | 7: 2682743 |
| 35613 | 7: 2682770 | 35614 | 7: 2682781 | 35615 | 7: 2682785 | 35618 | 7: 2721521 | 35619 | 7: 2728053 |
| 35622 | 7: 2823155 | 35624 | 7: 2880427 | 35629 | 7: 2957250 | 35631 | 7: 3004323 | 35641 | 7: 3142936 |
| 35643 | 7: 3239293 | 35670 | 7: 3679709 | 35671 | 7: 3679809 | 35672 | 7: 3686101 | 35673 | 7: 3687496 |
| 35683 | 7: 3876358 | 35687 | 7: 3947888 | 35688 | 7: 3949113 | 35689 | 7: 3953770 | 35690 | 7: 3965550 |
| 35692 | 7: 4018660 | 35693 | 7: 4057873 | 35700 | 7: 4183821 | 35702 | 7: 4203677 | 35704 | 7: 4226758 |
| 35706 | 7: 4321618 | 35708 | 7: 4425609 | 35709 | 7: 4432045 | 35713 | 7: 4509941 | 35714 | 7: 4530497 |
| 35715 | 7: 4550094 | 35724 | 7: 4890795 | 35725 | 7: 4931147 | 35733 | 7: 5056411 | 35734 | 7: 5076528 |
| 35735 | 7: 5077856 | 35736 | 7: 5077899 | 35737 | 7: 5077986 | 35738 | 7: 5078073 | 35743 | 7: 5135884 |
| 35744 | 7: 5135971 | 35747 | 7: 5227598 | 35758 | 7: 5539362 | 35759 | 7: 5540415 | 35760 | 7: 5541785 |
| 35761 | 7: 5542691 | 35762 | 7: 5543242 | 35763 | 7: 5543903 | 35764 | 7: 5544868 | 35765 | 7: 5545851 |
| 35766 | 7: 5546627 | 35767 | 7: 5547659 | 35768 | 7: 5548421 | 35769 | 7: 5554183 | 35770 | 7: 5554705 |
| 35771 | 7: 5555278 | 35772 | 7: 5556329 | 35773 | 7: 5560431 | 35775 | 7: 5587078 | 35781 | 7: 5670138 |
| 35783 | 7: 5725687 | 35784 | 7: 5728200 | 35787 | 7: 5774079 | 35788 | 7: 5775685 | 35789 | 7: 5775738 |
| 35790 | 7: 5775833 | 35791 | 7: 5775864 | 35794 | 7: 5854438 | 35795 | 7: 5867893 | 35796 | 7: 5869641 |
| 35797 | 7: 5875629 | 35798 | 7: 5876835 | 35799 | 7: 5879220 | 35800 | 7: 5884897 | 35801 | 7: 5888476 |
| 35803 | 7: 5895045 | 35804 | 7: 5905064 | 35807 | 7: 5948030 | 35839 | 7: 6133269 | 35848 | 7: 6256347 |
| 35850 | 7: 6276752 | 35852 | 7: 6318895 | 35853 | 7: 6318915 | 35867 | 7: 6376467 | 35868 | 7: 6389582 |
| 35877 | 7: 6654578 | 35885 | 7: 6739561 | 35892 | 7: 6820748 | 35896 | 7: 6837253 | 35905 | 7: 7044892 |
| 35906 | 7: 7044893 | 35909 | 7: 7081346 | 35912 | 7: 7131477 | 35913 | 7: 7131577 | 35979 | 7: 9765475 |
| 35998 | 7: 10641237 | 36009 | 7: 11092130 | 36015 | 7: 11145935 | 36016 | 7: 11154819 | 36019 | 7: 11233180 |
| 36020 | 7: 11233207 | 36021 | 7: 11237049 | 36022 | 7: 11296724 | 36023 | 7: 11306644 | 36024 | 7: 11332158 |
| 36026 | 7: 11394630 | 36027 | 7: 11394736 | 36028 | 7: 11403649 | 36042 | 7: 11870658 | 36043 | 7: 11885250 |
| 36062 | 7: 12227428 | 36070 | 7: 12344743 | 36071 | 7: 12344879 | 36082 | 7: 12522109 | 36088 | 7: 12587487 |
| 36093 | 7: 12738904 | 36096 | 7: 12762542 | 36097 | 7: 12763942 | 36099 | 7: 12806635 | 36100 | 7: 12816543 |
| 36102 | 7: 12824241 | 36103 | 7: 12852855 | 36106 | 7: 12896445 | 36107 | 7: 12897971 | 36108 | 7: 12898039 |
| 36109 | 7: 12898062 | 36111 | 7: 12974218 | 36112 | 7: 12974361 | 36115 | 7: 13016195 | 36121 | 7: 13189985 |
| 36122 | 7: 13201701 | 36123 | 7: 13204223 | 36124 | 7: 13204306 | 36125 | 7: 13209001 | 36126 | 7: 13209008 |
| 36127 | 7: 13209051 | 36128 | 7: 13209093 | 36129 | 7: 13211564 | 36130 | 7: 13211698 | 36131 | 7: 13211746 |
| 36133 | 7: 13234671 | 36135 | 7: 13241201 | 36136 | 7: 13248867 | 36137 | 7: 13252997 | 36145 | 7: 13436247 |
| 36160 | 7: 13763740 | 36161 | 7: 13764174 | 36163 | 7: 13826339 | 36164 | 7: 13826607 | 36165 | 7: 13847719 |
| 36166 | 7: 13847770 | 36167 | 7: 13848168 | 36169 | 7: 13907560 | 36170 | 7: 13907747 | 36171 | 7: 13908190 |
| 36175 | 7: 13970540 | 36176 | 7: 13970551 | 36177 | 7: 13971039 | 36182 | 7: 14105363 | 36187 | 7: 14216180 |
| 36188 | 7: 14278452 | 36196 | 7: 14568342 | 36197 | 7: 14569870 | 36198 | 7: 14569910 | 36199 | 7: 14570835 |
| 36200 | 7: 14571116 | 36201 | 7: 14571263 | 36202 | 7: 14572010 | 36203 | 7: 14572312 | 36204 | 7: 14574790 |
| 36205 | 7: 14574794 | 36206 | 7: 14574824 | 36207 | 7: 14574906 | 36208 | 7: 14575332 | 36209 | 7: 14575373 |
| 36210 | 7: 14576408 | 36211 | 7: 14576429 | 36212 | 7: 14576444 | 36213 | 7: 14577168 | 36214 | 7: 14577625 |
| 36215 | 7: 14578417 | 36216 | 7: 14578461 | 36217 | 7: 14578495 | 36218 | 7: 14587075 | 36221 | 7: 14674502 |
| 36222 | 7: 14674589 | 36224 | 7: 14688808 | 36225 | 7: 14689037 | 36226 | 7: 14690346 | 36227 | 7: 14690813 |
| 36228 | 7: 14692758 | 36229 | 7: 14695086 | 36230 | 7: 14790790 | 36231 | 7: 14846055 | 36232 | 7: 14847442 |
| 36241 | 7: 15055421 | 36242 | 7: 15055460 | 36246 | 7: 15113333 | 36247 | 7: 15116085 | 36248 | 7: 15116605 |
| 36249 | 7: 15120550 | 36250 | 7: 15121347 | 36251 | 7: 15121459 | 36252 | 7: 15131143 | 36254 | 7: 15149591 |
| 36255 | 7: 15158037 | 36257 | 7: 15188314 | 36258 | 7: 15219440 | 36262 | 7: 15240130 | 36267 | 7: 15342910 |
| 36269 | 7: 15386114 | 36273 | 7: 15463697 | 36274 | 7: 15501033 | 36283 | 7: 15718347 | 36297 | 7: 16146481 |
| 36298 | 7: 16189628 | 36300 | 7: 16237783 | 36305 | 7: 16292747 | 36313 | 7: 16521674 | 36314 | 7: 16522621 |
| 36315 | 7: 16524699 | 36317 | 7: 16531552 | 36318 | 7: 16534211 | 36319 | 7: 16535641 | 36323 | 7: 16561541 |
| 36359 | 7: 17538355 | 36366 | 7: 17643702 | 36367 | 7: 17694868 | 36368 | 7: 17698407 | 36369 | 7: 17698410 |
| 36371 | 7: 17712921 | 36389 | 7: 18292860 | 36395 | 7: 18386812 | 36397 | 7: 18404440 | 36398 | 7: 18434206 |
| 36399 | 7: 18443286 | 36401 | 7: 18446615 | 36402 | 7: 18464757 | 36408 | 7: 18658963 | 36411 | 7: 18704573 |
| 36412 | 7: 18706088 | 36413 | 7: 18777233 | 36414 | 7: 18826386 | 36417 | 7: 18862062 | 36424 | 7: 19108968 |
| 36425 | 7: 19113510 | 36426 | 7: 19115360 | 36428 | 7: 19141191 | 36431 | 7: 19189844 | 36436 | 7: 19259287 |
| 36437 | 7: 19261276 | 36438 | 7: 19270709 | 36439 | 7: 19272732 | 36443 | 7: 19317767 | 36446 | 7: 19337660 |
| 36449 | 7: 19501368 | 36451 | 7: 19560991 | 36452 | 7: 19570534 | 36453 | 7: 19601934 | 36454 | 7: 19624357 |
| 36455 | 7: 19629075 | 36456 | 7: 19634135 | 36457 | 7: 19674365 | 36459 | 7: 19694033 | 36460 | 7: 19709157 |
| 36462 | 7: 19723388 | 36463 | 7: 19742259 | 36464 | 7: 19745894 | 36465 | 7: 19749906 | 36468 | 7: 19770884 |
| 36469 | 7: 19782434 | 36470 | 7: 19795534 | 36471 | 7: 19811128 | 36473 | 7: 19832477 | 36474 | 7: 19846412 |
| 36475 | 7: 19854110 | 36476 | 7: 19861073 | 36477 | 7: 19870302 | 36478 | 7: 19874067 | 36479 | 7: 19877997 |
| 36480 | 7: 19882846 | 36481 | 7: 19900064 | 36482 | 7: 19904145 | 36483 | 7: 19920619 | 36484 | 7: 19925859 |
| 36485 | 7: 19929090 | 36486 | 7: 19934553 | 36487 | 7: 19940802 | 36488 | 7: 19948369 | 36489 | 7: 19954948 |
| 36490 | 7: 19962302 | 36491 | 7: 19974241 | 36492 | 7: 19977349 | 36494 | 7: 20018689 | 36495 | 7: 20036738 |
| 36496 | 7: 20053188 | 36497 | 7: 20057534 | 36498 | 7: 20076160 | 36507 | 7: 20236317 | 36512 | 7: 20288732 |
| 36513 | 7: 20318995 | 36514 | 7: 20324208 | 36515 | 7: 20324611 | 36517 | 7: 20355414 | 36518 | 7: 20356311 |
| 36519 | 7: 20356363 | 36520 | 7: 20356383 | 36521 | 7: 20366443 | 36522 | 7: 20366466 | 36531 | 7: 20582777 |
| 36532 | 7: 20591858 | 36534 | 7: 20634478 | 36541 | 7: 20805581 | 36543 | 7: 20890886 | 36544 | 7: 20890946 |
| 36547 | 7: 20913247 | 36549 | 7: 20940338 | 36558 | 7: 21139922 | 36565 | 7: 21283821 | 36570 | 7: 21380240 |
| 36571 | 7: 21386399 | 36573 | 7: 21403549 | 36596 | 7: 21986701 | 36601 | 7: 22039959 | 36613 | 7: 22387715 |
| 36624 | 7: 22566377 | 36625 | 7: 22566448 | 36627 | 7: 22594692 | 36628 | 7: 22594727 | 36633 | 7: 22680039 |
| 36635 | 7: 22786294 | 36637 | 7: 22926260 | 36645 | 7: 23219383 | 36648 | 7: 23248102 | 36655 | 7: 23451699 |
| 36702 | 7: 24500311 | 36711 | 7: 24783223 | 36714 | 7: 24910871 | 36745 | 7: 25616363 | 36749 | 7: 25654878 |
| 36752 | 7: 25731016 | 36753 | 7: 25768089 | 36757 | 7: 25800631 | 36758 | 7: 25832593 | 36764 | 7: 25936782 |
| 36766 | 7: 26024639 | 36777 | 7: 26292161 | 36778 | 7: 26308990 | 36779 | 7: 26319916 | 36782 | 7: 26387075 |
| 36784 | 7: 26402172 | 36788 | 7: 26497485 | 36790 | 7: 26557163 | 36791 | 7: 26574444 | 36792 | 7: 26577081 |
| 36793 | 7: 26577087 | 36794 | 7: 26578715 | 36796 | 7: 26594068 | 36797 | 7: 26594073 | 36798 | 7: 26594090 |
| 36801 | 7: 26638130 | 36805 | 7: 26769987 | 36806 | 7: 26769998 | 36807 | 7: 26770016 | 36811 | 7: 26823826 |
| 36814 | 7: 26881081 | 36815 | 7: 26898868 | 36821 | 7: 27031629 | 36825 | 7: 27060226 | 36832 | 7: 27198824 |
| 36840 | 7: 27423246 | 36841 | 7: 27434826 | 36848 | 7: 27596371 | 36855 | 7: 27733288 | 36862 | 7: 27870224 |
| 36865 | 7: 28003332 | 36867 | 7: 28049997 | 36868 | 7: 28050030 | 36869 | 7: 28050091 | 36870 | 7: 28050098 |
| 36886 | 7: 28544979 | 36893 | 7: 28690143 | 36895 | 7: 28700180 | 36897 | 7: 28729276 | 36900 | 7: 28830783 |
| 36902 | 7: 28879641 | 36908 | 7: 29036081 | 36912 | 7: 29112331 | 36913 | 7: 29178982 | 36922 | 7: 29366546 |
| 36924 | 7: 29452732 | 36934 | 7: 29594179 | 36936 | 7: 29632089 | 36938 | 7: 29666350 | 36953 | 7: 29976577 |
| 36956 | 7: 30094509 | 36968 | 7: 30358105 | 36971 | 7: 30403065 | 36974 | 7: 30553468 | 36977 | 7: 30644004 |
| 36981 | 7: 30714382 | 36982 | 7: 30714446 | 36983 | 7: 30756401 | 36984 | 7: 30807818 | 36988 | 7: 30863698 |
| 36992 | 7: 30906898 | 36995 | 7: 30991586 | 36998 | 7: 31107264 | 37025 | 7: 31626205 | 37029 | 7: 31642354 |
| 37030 | 7: 31642496 | 37031 | 7: 31643684 | 37032 | 7: 31643707 | 37037 | 7: 31676529 | 37039 | 7: 31699035 |
| 37065 | 7: 32314617 | 37080 | 7: 32688342 | 37081 | 7: 32709556 | 37085 | 7: 32796768 | 37096 | 7: 33201093 |
| 37097 | 7: 33201168 | 37119 | 7: 33724498 | 37122 | 7: 33761341 | 37123 | 7: 33814744 | 37124 | 7: 33814750 |
| 37125 | 7: 33814773 | 37126 | 7: 33814812 | 37129 | 7: 33866953 | 37153 | 7: 34486136 | 37154 | 7: 34502493 |
| 37163 | 7: 34781679 | 37170 | 7: 34951198 | 37172 | 7: 34967857 | 37173 | 7: 34967898 | 37176 | 7: 35028517 |
| 37182 | 7: 35229836 | 37199 | 7: 35718668 | 37219 | 7: 36283231 | 37220 | 7: 36308740 | 37231 | 7: 36510620 |
| 37238 | 7: 36626658 | 37239 | 7: 36666622 | 37265 | 7: 37451557 | 37266 | 7: 37459216 | 37269 | 7: 37490139 |
| 37273 | 7: 37534315 | 37277 | 7: 37590371 | 37281 | 7: 37620741 | 37287 | 7: 37840929 | 37291 | 7: 37934995 |
| 37297 | 7: 38048904 | 37298 | 7: 38081521 | 37299 | 7: 38087100 | 37300 | 7: 38093801 | 37301 | 7: 38101864 |
| 37302 | 7: 38124565 | 37303 | 7: 38128610 | 37304 | 7: 38129977 | 37314 | 7: 38325007 | 37316 | 7: 38360533 |
| 37317 | 7: 38365253 | 37318 | 7: 38389751 | 37321 | 7: 38404339 | 37322 | 7: 38421353 | 37323 | 7: 38429558 |
| 37327 | 7: 38515000 | 37328 | 7: 38534271 | 37333 | 7: 38753839 | 37338 | 7: 38815021 | 37343 | 7: 38952074 |
| 37355 | 7: 39351372 | 37356 | 7: 39362501 | 37359 | 7: 39455426 | 37368 | 7: 39695685 | 37372 | 7: 39721571 |
| 37373 | 7: 39721626 | 37381 | 7: 40016354 | 37385 | 7: 40095095 | 37388 | 7: 40114076 | 37389 | 7: 40149307 |
| 37390 | 7: 40151612 | 37396 | 7: 40294613 | 37397 | 7: 40294728 | 37398 | 7: 40294729 | 37399 | 7: 40294731 |
| 37400 | 7: 40294744 | 37401 | 7: 40294745 | 37404 | 7: 40323542 | 37407 | 7: 40355854 | 37408 | 7: 40360599 |
| 37409 | 7: 40361142 | 37410 | 7: 40361184 | 37411 | 7: 40361244 | 37412 | 7: 40361336 | 37413 | 7: 40372170 |
| 37414 | 7: 40372221 | 37415 | 7: 40385848 | 37416 | 7: 40441629 | 37419 | 7: 40507729 | 37420 | 7: 40508207 |
| 37423 | 7: 40603077 | 37424 | 7: 40603099 | 37425 | 7: 40604704 | 37426 | 7: 40605637 | 37427 | 7: 40606059 |
| 37429 | 7: 40622112 | 37436 | 7: 40917929 | 37438 | 7: 40932803 | 37439 | 7: 40933182 | 37440 | 7: 40933299 |
| 37441 | 7: 40945845 | 37442 | 7: 40946121 | 37443 | 7: 40946770 | 37444 | 7: 40957931 | 37446 | 7: 40958025 |
| 37447 | 7: 40960932 | 37453 | 7: 41290614 | 37454 | 7: 41322935 | 37461 | 7: 41552605 | 37462 | 7: 41552746 |
| 37463 | 7: 41553267 | 37464 | 7: 41553442 | 37467 | 7: 41694074 | 37468 | 7: 41694108 | 37470 | 7: 41791738 |
| 37472 | 7: 41828300 | 37473 | 7: 41828310 | 37474 | 7: 41828476 | 37475 | 7: 41828489 | 37476 | 7: 41828529 |
| 37477 | 7: 41828531 | 37478 | 7: 41828618 | 37483 | 7: 42132719 | 37495 | 7: 42627355 | 37496 | 7: 42628923 |
| 37498 | 7: 42659874 | 37499 | 7: 42659928 | 37509 | 7: 43070478 | 37510 | 7: 43070495 | 37511 | 7: 43073489 |
| 37512 | 7: 43074460 | 37513 | 7: 43074485 | 37514 | 7: 43075276 | 37515 | 7: 43075327 | 37516 | 7: 43075329 |
| 37517 | 7: 43075823 | 37518 | 7: 43075911 | 37519 | 7: 43077087 | 37522 | 7: 43114270 | 37525 | 7: 43247821 |
| 37526 | 7: 43258641 | 37531 | 7: 43495660 | 37532 | 7: 43504923 | 37533 | 7: 43504924 | 37534 | 7: 43509552 |
| 37535 | 7: 43518518 | 37536 | 7: 43526310 | 37537 | 7: 43541646 | 37539 | 7: 43547038 | 37540 | 7: 43551313 |
| 37541 | 7: 43573739 | 37542 | 7: 43602807 | 37543 | 7: 43616877 | 37546 | 7: 43695498 | 37548 | 7: 43809877 |
| 37549 | 7: 43811263 | 37550 | 7: 43812894 | 37552 | 7: 43875604 | 37558 | 7: 44162642 | 37563 | 7: 44416303 |
| 37566 | 7: 44610929 | 37568 | 7: 44639553 | 37569 | 7: 44666191 | 37572 | 7: 44764045 | 37573 | 7: 44764061 |
| 37574 | 7: 44789031 | 37575 | 7: 44789067 | 37576 | 7: 44789088 | 37584 | 7: 44943643 | 37585 | 7: 45003392 |
| 37591 | 7: 45101749 | 37599 | 7: 45350646 | 37600 | 7: 45355058 | 37605 | 7: 45445789 | 37614 | 7: 45661047 |
| 37619 | 7: 45951627 | 37620 | 7: 45994439 | 37621 | 7: 45997784 | 37623 | 7: 46123501 | 37624 | 7: 46133078 |
| 37637 | 7: 46424169 | 37649 | 7: 46722010 | 37655 | 7: 46847399 | 37657 | 7: 46881748 | 37659 | 7: 46963800 |
| 37660 | 7: 46965141 | 37661 | 7: 46965986 | 37662 | 7: 46966917 | 37663 | 7: 46969143 | 37678 | 7: 47071431 |
| 37680 | 7: 47200029 | 37694 | 7: 47663425 | 37696 | 7: 47675422 | 37697 | 7: 47681261 | 37701 | 7: 47796975 |
| 37722 | 7: 48566329 | 37730 | 7: 48673881 | 37771 | 7: 50021166 | 37780 | 7: 50639584 | 37792 | 7: 51149038 |
| 37796 | 7: 51377867 | 37798 | 7: 51400935 | 37801 | 7: 51429431 | 37803 | 7: 51450812 | 37811 | 7: 51923554 |
| 37812 | 7: 52003650 | 37815 | 7: 52173201 | 37817 | 7: 52176528 | 37818 | 7: 52177126 | 37819 | 7: 52179498 |
| 37820 | 7: 52181770 | 37821 | 7: 52182473 | 37822 | 7: 52183049 | 37823 | 7: 52183519 | 37824 | 7: 52186624 |
| 37825 | 7: 52193152 | 37826 | 7: 52196823 | 37827 | 7: 52197432 | 37828 | 7: 52198757 | 37829 | 7: 52199982 |
| 37830 | 7: 52207035 | 37831 | 7: 52210371 | 37833 | 7: 52224828 | 37834 | 7: 52225521 | 37835 | 7: 52227549 |
| 37836 | 7: 52227592 | 37838 | 7: 52249785 | 37839 | 7: 52251276 | 37840 | 7: 52251297 | 37841 | 7: 52251426 |
| 37842 | 7: 52251546 | 37843 | 7: 52251618 | 37844 | 7: 52251861 | 37845 | 7: 52252987 | 37846 | 7: 52263699 |
| 37847 | 7: 52264079 | 37848 | 7: 52264430 | 37849 | 7: 52264511 | 37850 | 7: 52264761 | 37851 | 7: 52266394 |
| 37852 | 7: 52282234 | 37853 | 7: 52289509 | 37854 | 7: 52291347 | 37855 | 7: 52293091 | 37856 | 7: 52294120 |
| 37857 | 7: 52295099 | 37858 | 7: 52296408 | 37859 | 7: 52296468 | 37860 | 7: 52298384 | 37861 | 7: 52299162 |
| 37862 | 7: 52300813 | 37863 | 7: 52303764 | 37864 | 7: 52305125 | 37865 | 7: 52305218 | 37866 | 7: 52308210 |
| 37867 | 7: 52308534 | 37868 | 7: 52308687 | 37869 | 7: 52309821 | 37870 | 7: 52339995 | 37871 | 7: 52344653 |
| 37872 | 7: 52345469 | 37873 | 7: 52348265 | 37874 | 7: 52349069 | 37878 | 7: 52567577 | 37879 | 7: 52610395 |
| 37880 | 7: 52611688 | 37884 | 7: 52667909 | 37895 | 7: 52931391 | 37898 | 7: 53021130 | 3790 | 7: 53089284 |
| 37908 | 7: 53195304 | 37920 | 7: 53551894 | 37923 | 7: 53581842 | 37924 | 7: 53587134 | 37928 | 7: 53866382 |
| 37931 | 7: 53935965 | 37934 | 7: 53979466 | 37938 | 7: 54160608 | 37943 | 7: 54227894 | 37944 | 7: 54238880 |
| 37949 | 7: 54401837 | 37950 | 7: 54414044 | 37952 | 7: 54475662 | 37983 | 7: 55275367 | 37984 | 7: 55310804 |
| 37992 | 7: 55508633 | 37997 | 7: 55613829 | 38014 | 7: 56033788 | 38027 | 7: 56270767 | 38054 | 7: 57224558 |
| 38056 | 7: 57261914 | 38060 | 7: 57381076 | 38067 | 7: 57701637 | 38068 | 7: 57702653 | 38079 | 7: 57942576 |
| 38099 | 7: 58504274 | 38100 | 7: 58564407 | 38103 | 7: 58594636 | 38106 | 7: 58736129 | 38112 | 7: 58873109 |
| 38114 | 7: 58937938 | 38118 | 7: 59041978 | 38119 | 7: 59042119 | 38120 | 7: 59044072 | 38121 | 7: 59055433 |
| 38123 | 7: 59057188 | 38124 | 7: 59057293 | 38127 | 7: 59101278 | 38128 | 7: 59111622 | 38132 | 7: 59143825 |
| 38141 | 7: 59349638 | 38142 | 7: 59349648 | 38143 | 7: 59357865 | 38144 | 7: 59366049 | 38147 | 7: 59372999 |
| 38148 | 7: 59373109 | 38149 | 7: 59373145 | 38150 | 7: 59373890 | 38151 | 7: 59373911 | 38152 | 7: 59373987 |
| 38154 | 7: 59405369 | 38161 | 7: 59476011 | 38173 | 7: 59813378 | 38174 | 7: 59851233 | 38176 | 7: 59880943 |
| 38179 | 7: 59979404 | 38180 | 7: 59993171 | 38183 | 7: 60051047 | 38187 | 7: 60113080 | 38190 | 7: 60121204 |
| 38191 | 7: 60146715 | 38192 | 7: 60159491 | 38202 | 7: 60502888 | 38205 | 7: 60568272 | 38213 | 7: 60834283 |
| 38220 | 7: 60976830 | 38222 | 7: 60986666 | 38223 | 7: 60989492 | 38225 | 7: 61027791 | 38226 | 7: 61067590 |
| 38227 | 7: 61075693 | 38230 | 7: 61121888 | 38231 | 7: 61125177 | 38234 | 7: 61147314 | 38235 | 7: 61157706 |
| 38236 | 7: 61175816 | 38239 | 7: 61222597 | 38241 | 7: 61229844 | 38242 | 7: 61229883 | 38243 | 7: 61234323 |
| 38244 | 7: 61234418 | 38245 | 7: 61240972 | 38250 | 7: 61334946 | 38251 | 7: 61352203 | 38252 | 7: 61373465 |
| 38255 | 7: 61436635 | 38262 | 7: 61491892 | 38265 | 7: 61518179 | 38267 | 7: 61555353 | 38271 | 7: 61582159 |
| 38275 | 7: 61778550 | 38282 | 7: 62019992 | 38284 | 7: 62026620 | 38292 | 7: 62285057 | 38294 | 7: 62296401 |
| 38298 | 7: 62370721 | 38300 | 7: 62436131 | 38302 | 7: 62469301 | 38303 | 7: 62475081 | 38304 | 7: 62508971 |
| 38307 | 7: 62555449 | 38310 | 7: 62601203 | 38311 | 7: 62614056 | 38315 | 7: 62677859 | 38329 | 7: 62720115 |
| 38340 | 7: 62942190 | 38354 | 7: 63240676 | 38365 | 7: 63685100 | 38378 | 7: 63974136 | 38389 | 7: 64332584 |
| 38410 | 7: 64916386 | 38416 | 7: 65002376 | 38419 | 7: 65066376 | 38421 | 7: 65074340 | 38433 | 7: 65274641 |
| 38435 | 7: 65330563 | 38439 | 7: 65403716 | 38444 | 7: 65453830 | 38453 | 7: 65721041 | 38464 | 7: 65972504 |
| 38466 | 7: 65984655 | 38467 | 7: 66008306 | 38468 | 7: 66011532 | 38471 | 7: 66093673 | 38472 | 7: 66101631 |
| 38473 | 7: 66102398 | 38474 | 7: 66104176 | 38475 | 7: 66105023 | 38476 | 7: 66105843 | 38477 | 7: 66108875 |
| 38478 | 7: 66109426 | 38479 | 7: 66110363 | 38480 | 7: 66112663 | 38481 | 7: 66113133 | 38482 | 7: 66114775 |
| 38483 | 7: 66117158 | 38484 | 7: 66117914 | 38485 | 7: 66118467 | 38486 | 7: 66120071 | 38487 | 7: 66121243 |
| 38488 | 7: 66122354 | 38489 | 7: 66122876 | 38490 | 7: 66124410 | 38491 | 7: 66125761 | 38492 | 7: 66126649 |
| 38493 | 7: 66130675 | 38494 | 7: 66134907 | 38495 | 7: 66146626 | 38496 | 7: 66158927 | 38512 | 7: 66629354 |
| 38513 | 7: 66638097 | 38514 | 7: 66640996 | 38519 | 7: 66842880 | 38523 | 7: 66907257 | 38532 | 7: 67112605 |
| 38540 | 7: 67368109 | 38549 | 7: 67650949 | 38550 | 7: 67663732 | 38551 | 7: 67677225 | 38556 | 7: 67803883 |
| 38582 | 7: 68478076 | 38595 | 7: 68892260 | 38597 | 7: 68928960 | 38598 | 7: 68949630 | 38601 | 7: 68987039 |
| 38602 | 7: 68998753 | 38604 | 7: 69001013 | 38606 | 7: 69049520 | 38611 | 7: 69144296 | 38615 | 7: 69172516 |
| 38618 | 7: 69198573 | 38624 | 7: 69399622 | 38627 | 7: 69502402 | 38628 | 7: 69503500 | 38631 | 7: 69517359 |
| 38645 | 7: 69896069 | 38649 | 7: 69988788 | 38652 | 7: 70030654 | 38654 | 7: 70060807 | 38660 | 7: 70128127 |
| 38675 | 7: 70701637 | 38676 | 7: 70732259 | 38692 | 7: 71222059 | 38695 | 7: 71353347 | 38704 | 7: 71545001 |
| 38705 | 7: 71558907 | 38733 | 7: 72053452 | 38737 | 7: 72207894 | 38738 | 7: 72232062 | 38744 | 7: 72362246 |
| 38759 | 7: 72898450 | 38762 | 7: 72962658 | 38770 | 7: 73184647 | 38775 | 7: 73473535 | 38793 | 7: 73942821 |
| 38805 | 7: 74306314 | 38806 | 7: 74306327 | 38807 | 7: 74306379 | 38809 | 7: 74321645 | 38818 | 7: 74667948 |
| 38824 | 7: 74772424 | 38825 | 7: 74834470 | 38827 | 7: 74855482 | 38828 | 7: 74855556 | 38829 | 7: 74861008 |
| 38834 | 7: 74922579 | 38835 | 7: 74934509 | 38836 | 7: 74967965 | 38838 | 7: 75008858 | 38839 | 7: 75009945 |
| 38844 | 7: 75141363 | 38845 | 7: 75145201 | 38846 | 7: 75157547 | 38848 | 7: 75201764 | 38860 | 7: 75609551 |
| 38862 | 7: 75636089 | 38863 | 7: 75677642 | 38864 | 7: 75677689 | 38871 | 7: 75802119 | 38872 | 7: 75814579 |
| 38877 | 7: 75942645 | 38886 | 7: 76249772 | 38887 | 7: 76260400 | 38888 | 7: 76279595 | 38890 | 7: 76306268 |
| 38891 | 7: 76342697 | 38892 | 7: 76347497 | 38894 | 7: 76370749 | 38898 | 7: 76449574 | 38910 | 7: 76725621 |
| 38911 | 7: 76765051 | 38914 | 7: 76875833 | 38915 | 7: 76875983 | 38919 | 7: 77004376 | 38939 | 7: 77408753 |
| 38960 | 7: 77903085 | 38966 | 7: 77985310 | 38970 | 7: 78110195 | 38974 | 7: 78196572 | 38975 | 7: 78196650 |
| 38977 | 7: 78217247 | 39034 | 7: 79217925 | 39044 | 7: 79409160 | 39050 | 7: 79575037 | 39051 | 7: 79575100 |
| 39055 | 7: 79623429 | 39057 | 7: 79676699 | 39061 | 7: 79735833 | 39062 | 7: 79735898 | 39070 | 7: 79930582 |
| 39071 | 7: 79953397 | 39074 | 7: 80074984 | 39081 | 7: 80181414 | 39086 | 7: 80322587 | 39091 | 7: 80397326 |
| 39092 | 7: 80423038 | 39094 | 7: 80460624 | 39095 | 7: 80461082 | 39097 | 7: 80469480 | 39098 | 7: 80471094 |
| 39100 | 7: 80478643 | 39101 | 7: 80503605 | 39102 | 7: 80503765 | 39104 | 7: 80509969 | 39105 | 7: 80514758 |
| 39109 | 7: 80546745 | 39113 | 7: 80594054 | 39116 | 7: 80662788 | 39130 | 7: 81016584 | 39138 | 7: 81135163 |
| 39139 | 7: 81142008 | 39140 | 7: 81142055 | 39141 | 7: 81142172 | 39142 | 7: 81174306 | 39144 | 7: 81184476 |
| 39145 | 7: 81184486 | 39151 | 7: 81313732 | 39158 | 7: 81392597 | 39159 | 7: 81392623 | 39160 | 7: 81392634 |
| 39161 | 7: 81392650 | 39162 | 7: 81392653 | 39163 | 7: 81392665 | 39170 | 7: 81654928 | 39175 | 7: 81798512 |
| 39186 | 7: 82216490 | 39187 | 7: 82219959 | 39189 | 7: 82230892 | 39190 | 7: 82248295 | 39198 | 7: 82549435 |
| 39200 | 7: 82554305 | 39206 | 7: 82662900 | 39207 | 7: 82662928 | 39208 | 7: 82662929 | 39214 | 7: 82764185 |
| 39224 | 7: 83044183 | 39244 | 7: 83544032 | 39264 | 7: 84108168 | 39265 | 7: 84108203 | 39266 | 7: 84108212 |
| 39267 | 7: 84118928 | 39269 | 7: 84144485 | 39273 | 7: 84207697 | 39275 | 7: 84239228 | 39279 | 7: 84354399 |
| 39280 | 7: 84354448 | 39284 | 7: 84424155 | 39285 | 7: 84450073 | 39288 | 7: 84509794 | 39301 | 7: 85123948 |
| 39307 | 7: 85293142 | 39308 | 7: 85293161 | 39309 | 7: 85293169 | 39323 | 7: 85845513 | 39324 | 7: 85845564 |
| 39325 | 7: 85875827 | 39328 | 7: 85965693 | 39337 | 7: 86289890 | 39338 | 7: 86289929 | 39340 | 7: 86348377 |
| 39341 | 7: 86348400 | 39345 | 7: 86462459 | 39348 | 7: 86544147 | 39349 | 7: 86566118 | 39353 | 7: 86638215 |
| 39379 | 7: 87401188 | 39388 | 7: 87608805 | 39389 | 7: 87608884 | 39390 | 7: 87608885 | 39391 | 7: 87608911 |
| 39394 | 7: 87779029 | 39395 | 7: 87841745 | 39403 | 7: 88117633 | 39411 | 7: 88379769 | 39419 | 7: 88587478 |
| 39427 | 7: 88790932 | 39428 | 7: 88796649 | 39433 | 7: 88942654 | 39435 | 7: 88966573 | 39436 | 7: 89035670 |
| 39440 | 7: 89116248 | 39445 | 7: 89148646 | 39446 | 7: 89184830 | 39450 | 7: 89247931 | 39454 | 7: 89369998 |
| 39478 | 7: 90049487 | 39483 | 7: 90127121 | 39486 | 7: 90166602 | 39487 | 7: 90166627 | 39488 | 7: 90167634 |
| 39496 | 7: 90329807 | 39511 | 7: 90598533 | 39514 | 7: 90688919 | 39523 | 7: 90779825 | 39558 | 7: 90831595 |
| 39565 | 7: 90845831 | 39596 | 7: 91489178 | 39611 | 7: 91726773 | 39653 | 7: 92762601 | 39671 | 7: 93433611 |
| 39679 | 7: 93718483 | 39680 | 7: 93727310 | 39682 | 7: 93774135 | 39693 | 7: 94215835 | 39694 | 7: 94269949 |
| 39699 | 7: 94382446 | 39703 | 7: 94439368 | 39708 | 7: 94574747 | 39717 | 7: 94849950 | 39718 | 7: 94854494 |
| 39719 | 7: 94854604 | 39720 | 7: 94854627 | 39724 | 7: 94945483 | 39729 | 7: 95020011 | 39730 | 7: 95021828 |
| 39733 | 7: 95079030 | 39735 | 7: 95116563 | 39736 | 7: 95158884 | 39743 | 7: 95405216 | 39754 | 7: 95645495 |
| 39767 | 7: 95958530 | 39771 | 7: 96074523 | 39772 | 7: 96087310 | 39775 | 7: 96098877 | 39778 | 7: 96109770 |
| 39781 | 7: 96127017 | 39782 | 7: 96127023 | 39783 | 7: 96127094 | 39784 | 7: 96140233 | 39785 | 7: 96140250 |
| 39786 | 7: 96140274 | 39787 | 7: 96140286 | 39788 | 7: 96140316 | 39789 | 7: 96140325 | 39790 | 7: 96146797 |
| 39791 | 7: 96152289 | 39793 | 7: 96169102 | 39806 | 7: 96180272 | 39807 | 7: 96180277 | 39808 | 7: 96180291 |
| 39810 | 7: 96196608 | 39811 | 7: 96196642 | 39813 | 7: 96202522 | 39829 | 7: 96220995 | 39831 | 7: 96221193 |
| 39868 | 7: 96278344 | 39911 | 7: 96314599 | 39912 | 7: 96315078 | 39934 | 7: 96355415 | 39939 | 7: 96370734 |
| 39941 | 7: 96387738 | 39950 | 7: 96555097 | 39952 | 7: 96601732 | 39954 | 7: 96613640 | 39985 | 7: 97508892 |
| 39986 | 7: 97508905 | 39988 | 7: 97541930 | 39990 | 7: 97603739 | 39991 | 7: 97629287 | 39997 | 7: 97747540 |
| 40003 | 7: 97959533 | 40019 | 7: 98300666 | 40020 | 7: 98348623 | 40021 | 7: 98348636 | 40028 | 7: 98543164 |
| 40048 | 7: 99313338 | 40070 | 7: 99845218 | 40072 | 7: 99865657 | 40075 | 7: 99914902 | 40082 | 7: 100079502 |
| 40083 | 7: 100082753 | 40086 | 7: 100118655 | 40087 | 7: 100118835 | 40088 | 7: 100136909 | 40089 | 7: 100144865 |
| 40098 | 7: 100358134 | 40099 | 7: 100395807 | 40116 | 7: 100797612 | 40129 | 7: 101254706 | 40133 | 7: 101358810 |
| 40139 | 7: 101563788 | 40142 | 7: 101606446 | 40143 | 7: 101606448 | 40147 | 7: 101667972 | 40153 | 7: 101808436 |
| 40154 | 7: 101857168 | 40157 | 7: 102004868 | 40164 | 7: 102285994 | 40173 | 7: 102580748 | 40174 | 7: 102582243 |
| 40175 | 7: 102585831 | 40176 | 7: 102589674 | 40177 | 7: 102594188 | 40178 | 7: 102599028 | 40179 | 7: 102609048 |
| 40181 | 7: 102620944 | 40192 | 7: 102888318 | 40196 | 7: 102988342 | 40198 | 7: 103035483 | 40200 | 7: 103047606 |
| 40219 | 7: 103414818 | 40280 | 7: 105164324 | 40292 | 7: 105387524 | 40298 | 7: 105535552 | 40322 | 7: 106096645 |
| 40326 | 7: 106249472 | 40327 | 7: 106254713 | 40348 | 7: 106643847 | 40351 | 7: 106742504 | 40352 | 7: 106742506 |
| 40360 | 7: 106848432 | 40362 | 7: 106869396 | 40369 | 7: 107016860 | 40370 | 7: 107023813 | 40373 | 7: 107095006 |
| 40376 | 7: 107124835 | 40383 | 7: 107308750 | 40403 | 7: 108184476 | 40407 | 7: 108325718 | 40408 | 7: 108337537 |
| 40418 | 7: 108563367 | 40431 | 7: 108821941 | 40433 | 7: 108869029 | 40439 | 7: 108994618 | 40441 | 7: 109029038 |
| 40442 | 7: 109029077 | 40443 | 7: 109029102 | 40458 | 7: 109275280 | 40480 | 7: 109999314 | 40498 | 8: 373283 |
| 40506 | 8: 643950 | 40510 | 8: 764642 | 40511 | 8: 780102 | 40514 | 8: 800236 | 40515 | 8: 816422 |
| 40517 | 8: 844855 | 40520 | 8: 882426 | 40521 | 8: 883861 | 40535 | 8: 1082935 | 40536 | 8: 1086523 |
| 40548 | 8: 1362737 | 40552 | 8: 1579017 | 40564 | 8: 1757679 | 40571 | 8: 1832243 | 40579 | 8: 1939311 |
| 40594 | 8: 2317585 | 40606 | 8: 2582570 | 40616 | 8: 3008685 | 40620 | 8: 3178994 | 40621 | 8: 3219275 |
| 40630 | 8: 3400647 | 40631 | 8: 3416328 | 40632 | 8: 3416882 | 40667 | 8: 4251182 | 40668 | 8: 4273155 |
| 40669 | 8: 4274587 | 40670 | 8: 4276578 | 40671 | 8: 4280150 | 40672 | 8: 4283775 | 40685 | 8: 4639116 |
| 40687 | 8: 4644380 | 40688 | 8: 4644486 | 40690 | 8: 4708141 | 40693 | 8: 4759903 | 40698 | 8: 4908669 |
| 40707 | 8: 5249662 | 40708 | 8: 5270683 | 40709 | 8: 5270703 | 40710 | 8: 5270802 | 40711 | 8: 5270815 |
| 40712 | 8: 5332706 | 40728 | 8: 5760796 | 40735 | 8: 5869022 | 40741 | 8: 6027760 | 40765 | 8: 6756792 |
| 40766 | 8: 6764623 | 40785 | 8: 7181322 | 40786 | 8: 7181335 | 40803 | 8: 7581528 | 40805 | 8: 7596757 |
| 40806 | 8: 7598915 | 40807 | 8: 7601383 | 40808 | 8: 7603103 | 40809 | 8: 7611610 | 40810 | 8: 7618608 |
| 40811 | 8: 7619933 | 40812 | 8: 7620831 | 40813 | 8: 7625351 | 40818 | 8: 7818361 | 40826 | 8: 8001917 |
| 40832 | 8: 8175029 | 40842 | 8: 8393306 | 40844 | 8: 8477216 | 40848 | 8: 8618577 | 40857 | 8: 8769353 |
| 40866 | 8: 9042180 | 40868 | 8: 9063209 | 40869 | 8: 9119208 | 40889 | 8: 9619039 | 40890 | 8: 9619107 |
| 40891 | 8: 9619135 | 40892 | 8: 9623038 | 40893 | 8: 9627034 | 40894 | 8: 9627109 | 40895 | 8: 9627275 |
| 40896 | 8: 9627321 | 40905 | 8: 9890341 | 40906 | 8: 9901901 | 40910 | 8: 10031350 | 40917 | 8: 10339279 |
| 40925 | 8: 10604669 | 40931 | 8: 10696791 | 40934 | 8: 10712286 | 40935 | 8: 10712485 | 40936 | 8: 10712536 |
| 40939 | 8: 10783394 | 40942 | 8: 10854210 | 40943 | 8: 10854709 | 40952 | 8: 11049154 | 40955 | 8: 11128689 |
| 40980 | 8: 11615963 | 40981 | 8: 11626767 | 40983 | 8: 11655158 | 40985 | 8: 11693820 | 40990 | 8: 11853891 |
| 41001 | 8: 12089230 | 41008 | 8: 12344836 | 41012 | 8: 12433144 | 41026 | 8: 12914075 | 41036 | 8: 13348241 |
| 41038 | 8: 13393062 | 41047 | 8: 13722063 | 41097 | 8: 15148225 | 41103 | 8: 15333923 | 41109 | 8: 15473403 |
| 41113 | 8: 15497145 | 41114 | 8: 15497203 | 41115 | 8: 15497248 | 41116 | 8: 15533442 | 41120 | 8: 15590887 |
| 41121 | 8: 15590953 | 41124 | 8: 15685122 | 41128 | 8: 15765706 | 41133 | 8: 15967637 | 41143 | 8: 16338822 |
| 41145 | 8: 16399613 | 41155 | 8: 16592732 | 41158 | 8: 16699848 | 41162 | 8: 16791579 | 41163 | 8: 16792851 |
| 41171 | 8: 16926726 | 41174 | 8: 16945156 | 41178 | 8: 17032464 | 41179 | 8: 17032475 | 41180 | 8: 17032488 |
| 41182 | 8: 17048252 | 41183 | 8: 17055738 | 41184 | 8: 17055749 | 41185 | 8: 17055775 | 41186 | 8: 17056869 |
| 41187 | 8: 17061563 | 41188 | 8: 17064508 | 41193 | 8: 17304347 | 41195 | 8: 17305220 | 41196 | 8: 17307048 |
| 41197 | 8: 17307131 | 41206 | 8: 17548023 | 41231 | 8: 18280322 | 41242 | 8: 18613707 | 41246 | 8: 18768280 |
| 41257 | 8: 19052433 | 41288 | 8: 19935582 | 41301 | 8: 20342622 | 41342 | 8: 21329932 | 41361 | 8: 21984030 |
| 41363 | 8: 22082837 | 41364 | 8: 22100760 | 41365 | 8: 22113575 | 41396 | 8: 23324599 | 41401 | 8: 23427337 |
| 41421 | 8: 24057660 | 41434 | 8: 24296988 | 41440 | 8: 24394806 | 41442 | 8: 24405999 | 41444 | 8: 24443570 |
| 41450 | 8: 24643364 | 41452 | 8: 24674588 | 41454 | 8: 24687934 | 41455 | 8: 24689851 | 41456 | 8: 24692069 |
| 41457 | 8: 24700212 | 41459 | 8: 24720840 | 41462 | 8: 24752172 | 41466 | 8: 24771365 | 41467 | 8: 24786333 |
| 41468 | 8: 24793655 | 41469 | 8: 24802374 | 41470 | 8: 24812615 | 41471 | 8: 24820473 | 41472 | 8: 24827262 |
| 41475 | 8: 24874038 | 41476 | 8: 24875382 | 41477 | 8: 24877159 | 41478 | 8: 24877700 | 41479 | 8: 24878235 |
| 41480 | 8: 24878922 | 41481 | 8: 24888678 | 41482 | 8: 24891103 | 41483 | 8: 24895594 | 41485 | 8: 24933323 |
| 41486 | 8: 24938771 | 41487 | 8: 24940757 | 41488 | 8: 24955657 | 41489 | 8: 24957890 | 41490 | 8: 24961237 |
| 41491 | 8: 24966416 | 41492 | 8: 24974478 | 41493 | 8: 24979200 | 41494 | 8: 24988001 | 41495 | 8: 24992186 |
| 41496 | 8: 24996358 | 41497 | 8: 25006948 | 41498 | 8: 25013387 | 41499 | 8: 25035664 | 41500 | 8: 25041072 |
| 41501 | 8: 25059424 | 41506 | 8: 25124953 | 41507 | 8: 25126729 | 41509 | 8: 25140406 | 41510 | 8: 25144505 |
| 41511 | 8: 25146183 | 41512 | 8: 25148717 | 41513 | 8: 25150222 | 41514 | 8: 25152059 | 41515 | 8: 25154593 |
| 41516 | 8: 25157388 | 41517 | 8: 25160394 | 41518 | 8: 25165311 | 41519 | 8: 25166943 | 41520 | 8: 25169477 |
| 41522 | 8: 25174754 | 41523 | 8: 25177709 | 41524 | 8: 25180230 | 41525 | 8: 25185692 | 41526 | 8: 25188439 |
| 41527 | 8: 25198273 | 41528 | 8: 25200755 | 41530 | 8: 25214312 | 41531 | 8: 25227176 | 41532 | 8: 25233627 |
| 41533 | 8: 25235124 | 41534 | 8: 25238388 | 41535 | 8: 25240252 | 41537 | 8: 25243485 | 41538 | 8: 25254140 |
| 41539 | 8: 25257480 | 41540 | 8: 25259038 | 41541 | 8: 25261263 | 41542 | 8: 25263852 | 41543 | 8: 25265485 |
| 41544 | 8: 25266937 | 41545 | 8: 25268844 | 41546 | 8: 25274693 | 41547 | 8: 25282832 | 41548 | 8: 25294627 |
| 41557 | 8: 25487377 | 41558 | 8: 25500162 | 41561 | 8: 25551702 | 41580 | 8: 25993221 | 41584 | 8: 26067879 |
| 41602 | 8: 26560396 | 41604 | 8: 26591338 | 41614 | 8: 26825483 | 41621 | 8: 26909158 | 41631 | 8: 27313786 |
| 41633 | 8: 27383296 | 41637 | 8: 27558131 | 41649 | 8: 27894769 | 41652 | 8: 27969801 | 41654 | 8: 28067472 |
| 41664 | 8: 28411601 | 41665 | 8: 28411658 | 41670 | 8: 28576597 | 41673 | 8: 28652470 | 41678 | 8: 28903569 |
| 41679 | 8: 28935823 | 41691 | 8: 29203761 | 41702 | 8: 29452933 | 41706 | 8: 29509148 | 41707 | 8: 29543012 |
| 41728 | 8: 29972472 | 41730 | 8: 30031385 | 41734 | 8: 30076221 | 41737 | 8: 30253112 | 41750 | 8: 30514176 |
| 41754 | 8: 30666192 | 41757 | 8: 30707536 | 41764 | 8: 30964101 | 41765 | 8: 30965820 | 41767 | 8: 31027893 |
| 41769 | 8: 31049984 | 41770 | 8: 31056192 | 41773 | 8: 31128535 | 41776 | 8: 31166613 | 41780 | 8: 31290392 |
| 41781 | 8: 31300884 | 41785 | 8: 31442029 | 41786 | 8: 31453763 | 41790 | 8: 31579785 | 41793 | 8: 31643340 |
| 41801 | 8: 31867242 | 41802 | 8: 31895156 | 41859 | 8: 33917004 | 41888 | 8: 34615024 | 41894 | 8: 34800931 |
| 41895 | 8: 34800977 | 41896 | 8: 34801009 | 41919 | 8: 35445047 | 41923 | 8: 35459817 | 41936 | 8: 35833781 |
| 41938 | 8: 35859948 | 41943 | 8: 35930089 | 41953 | 8: 36065706 | 41956 | 8: 36096463 | 41958 | 8: 36187712 |
| 41962 | 8: 36233237 | 41965 | 8: 36292654 | 41968 | 8: 36343251 | 41985 | 8: 36842382 | 41986 | 8: 36851180 |
| 41995 | 8: 37031806 | 41996 | 8: 37054784 | 41998 | 8: 37095387 | 42008 | 8: 37407050 | 42010 | 8: 37435996 |
| 42023 | 8: 37797692 | 42024 | 8: 37854305 | 42028 | 8: 37928176 | 42044 | 8: 38675399 | 42047 | 8: 38691615 |
| 42056 | 8: 39019428 | 42061 | 8: 39290623 | 42067 | 8: 39583278 | 42068 | 8: 39584890 | 42069 | 8: 39621719 |
| 42074 | 8: 39696110 | 42075 | 8: 39718793 | 42077 | 8: 39768968 | 42078 | 8: 39769005 | 42079 | 8: 39809755 |
| 42081 | 8: 39864901 | 42091 | 8: 40003850 | 42101 | 8: 40171823 | 42102 | 8: 40219628 | 42103 | 8: 40219668 |
| 42105 | 8: 40257959 | 42106 | 8: 40287413 | 42107 | 8: 40306339 | 42109 | 8: 40334191 | 42110 | 8: 40334321 |
| 42114 | 8: 40410096 | 42131 | 8: 40743770 | 42135 | 8: 40830870 | 42150 | 8: 41247220 | 42152 | 8: 41309293 |
| 42165 | 8: 41611046 | 42168 | 8: 41682500 | 42169 | 8: 41693466 | 42170 | 8: 41697104 | 42174 | 8: 41759986 |
| 42176 | 8: 41790111 | 42179 | 8: 41858426 | 42180 | 8: 41890619 | 42181 | 8: 41897019 | 42199 | 8: 42436416 |
| 42211 | 8: 42709975 | 42212 | 8: 42720374 | 42216 | 8: 42880518 | 42220 | 8: 42958668 | 42221 | 8: 42958711 |
| 42222 | 8: 42958836 | 42226 | 8: 43016661 | 42236 | 8: 43367984 | 42245 | 8: 43812387 | 42246 | 8: 43815004 |
| 42250 | 8: 43957564 | 42255 | 8: 43998136 | 42256 | 8: 43998213 | 42258 | 8: 44010856 | 42269 | 8: 44431429 |
| 42273 | 8: 44588721 | 42282 | 8: 44879292 | 42283 | 8: 44895474 | 42285 | 8: 44916012 | 42295 | 8: 45238924 |
| 42301 | 8: 45399787 | 42304 | 8: 45427010 | 42306 | 8: 45447403 | 42309 | 8: 45519975 | 42310 | 8: 45544435 |
| 42314 | 8: 45672590 | 42316 | 8: 45691728 | 42321 | 8: 45748288 | 42327 | 8: 45881905 | 42328 | 8: 45949542 |
| 42333 | 8: 46053215 | 42334 | 8: 46058853 | 42347 | 8: 46184772 | 42352 | 8: 46322367 | 42358 | 8: 46514754 |
| 42362 | 8: 46662471 | 42363 | 8: 46688442 | 42367 | 8: 46794717 | 42378 | 8: 47069272 | 42379 | 8: 47096555 |
| 42383 | 8: 47242828 | 42384 | 8: 47301441 | 42386 | 8: 47301545 | 42396 | 8: 47442795 | 42397 | 8: 47454300 |
| 42398 | 8: 47468132 | 42413 | 8: 47827950 | 42416 | 8: 47959914 | 42443 | 8: 48870474 | 42452 | 8: 48980291 |
| 42455 | 8: 49060228 | 42456 | 8: 49060267 | 42457 | 8: 49060352 | 42458 | 8: 49071203 | 42459 | 8: 49082742 |
| 42465 | 8: 49227434 | 42466 | 8: 49248066 | 42470 | 8: 49305124 | 42471 | 8: 49328971 | 42472 | 8: 49332342 |
| 42484 | 8: 49820121 | 42490 | 8: 50120835 | 42493 | 8: 50158365 | 42496 | 8: 50210739 | 42502 | 8: 50343730 |
| 42505 | 8: 50410404 | 42526 | 8: 51083850 | 42529 | 8: 51149379 | 42530 | 8: 51154605 | 42537 | 8: 51409152 |
| 42549 | 8: 51645019 | 42553 | 8: 51697635 | 42563 | 8: 51983681 | 42569 | 8: 52152405 | 42570 | 8: 52157895 |
| 42573 | 8: 52201603 | 42584 | 8: 52496347 | 42593 | 8: 52683991 | 42597 | 8: 52775082 | 42606 | 8: 53099576 |
| 42607 | 8: 53099649 | 42608 | 8: 53099684 | 42618 | 8: 53465873 | 42622 | 8: 53559314 | 42626 | 8: 53663149 |
| 42628 | 8: 53760217 | 42643 | 8: 54223148 | 42650 | 8: 54393899 | 42663 | 8: 54661982 | 42665 | 8: 54767805 |
| 42666 | 8: 54767855 | 42676 | 8: 54981068 | 42677 | 8: 54981074 | 42678 | 8: 54981156 | 42693 | 8: 55456387 |
| 42697 | 8: 55552471 | 42699 | 8: 55552575 | 42707 | 8: 55861711 | 42708 | 8: 55861771 | 42709 | 8: 55869592 |
| 42711 | 8: 55889240 | 42718 | 8: 56082094 | 42719 | 8: 56082109 | 42723 | 8: 56355997 | 42732 | 8: 56476308 |
| 42736 | 8: 56567532 | 42749 | 8: 56898409 | 42752 | 8: 56940553 | 42755 | 8: 57067743 | 42756 | 8: 57083802 |
| 42757 | 8: 57097646 | 42769 | 8: 57431848 | 42770 | 8: 57436089 | 42771 | 8: 57441513 | 42774 | 8: 57491864 |
| 42777 | 8: 57503925 | 42781 | 8: 57561608 | 42784 | 8: 57609698 | 42785 | 8: 57624471 | 42794 | 8: 58010694 |
| 42796 | 8: 58065599 | 42799 | 8: 58159003 | 42803 | 8: 58281685 | 42817 | 8: 58837870 | 42827 | 8: 59185961 |
| 42831 | 8: 59304332 | 42833 | 8: 59343826 | 42843 | 8: 59578341 | 42845 | 8: 59585372 | 42846 | 8: 59591203 |
| 42847 | 8: 59595742 | 42853 | 8: 59747078 | 42856 | 8: 59839831 | 42861 | 8: 59908902 | 42867 | 8: 59979908 |
| 42869 | 8: 59997834 | 42873 | 8: 60218203 | 42874 | 8: 60232849 | 42875 | 8: 60252660 | 42877 | 8: 60376601 |
| 42878 | 8: 60387912 | 42880 | 8: 60393687 | 42881 | 8: 60407147 | 42882 | 8: 60408080 | 42883 | 8: 60413868 |
| 42885 | 8: 60422237 | 42887 | 8: 60434309 | 42888 | 8: 60445473 | 42893 | 8: 60483449 | 42901 | 8: 60773154 |
| 42906 | 8: 60847978 | 42928 | 8: 61569513 | 42931 | 8: 61676872 | 42953 | 8: 62298007 | 42955 | 8: 62361352 |
| 42956 | 8: 62370401 | 42957 | 8: 62370423 | 42960 | 8: 62476415 | 42961 | 8: 62476489 | 42962 | 8: 62476593 |
| 42963 | 8: 62481551 | 42964 | 8: 62481563 | 42965 | 8: 62492081 | 42966 | 8: 62492171 | 42967 | 8: 62492207 |
| 42969 | 8: 62554727 | 42970 | 8: 62556949 | 42977 | 8: 62779236 | 42980 | 8: 62823371 | 42985 | 8: 62978731 |
| 42986 | 8: 62978820 | 42989 | 8: 63076318 | 42991 | 8: 63182958 | 42998 | 8: 63428149 | 43008 | 8: 63577043 |
| 43025 | 8: 64001632 | 43026 | 8: 64003121 | 43031 | 8: 64111218 | 43038 | 8: 64443505 | 43041 | 8: 64480556 |
| 43042 | 8: 64512625 | 43050 | 8: 64890322 | 43059 | 8: 65128134 | 43060 | 8: 65147458 | 43063 | 8: 65217066 |
| 43064 | 8: 65219496 | 43065 | 8: 65219567 | 43073 | 8: 65486798 | 43074 | 8: 65486879 | 43075 | 8: 65486964 |
| 43076 | 8: 65487030 | 43077 | 8: 65487045 | 43078 | 8: 65487167 | 43079 | 8: 65487520 | 43080 | 8: 65506614 |
| 43092 | 8: 66005380 | 43094 | 8: 66041697 | 43095 | 8: 66071027 | 43096 | 8: 66071753 | 43099 | 8: 66095067 |
| 43100 | 8: 66095107 | 43102 | 8: 66110177 | 43103 | 8: 66110963 | 43104 | 8: 66111005 | 43117 | 8: 66467235 |
| 43129 | 8: 66780866 | 43130 | 8: 66781633 | 43132 | 8: 66784915 | 43134 | 8: 66833433 | 43135 | 8: 66833492 |
| 43139 | 8: 66987197 | 43140 | 8: 66999877 | 43141 | 8: 67000007 | 43143 | 8: 67013017 | 43146 | 8: 67072138 |
| 43148 | 8: 67105385 | 43149 | 8: 67105408 | 43150 | 8: 67121705 | 43153 | 8: 67145134 | 43154 | 8: 67154528 |
| 43158 | 8: 67392786 | 43162 | 8: 67566051 | 43164 | 8: 67576272 | 43166 | 8: 67607555 | 43167 | 8: 67613943 |
| 43170 | 8: 67704512 | 43171 | 8: 67731230 | 43182 | 8: 67844754 | 43183 | 8: 67844773 | 43200 | 8: 68399674 |
| 43216 | 8: 68844096 | 43217 | 8: 68866548 | 43218 | 8: 68866614 | 43223 | 8: 68960840 | 43224 | 8: 69009944 |
| 43227 | 8: 69053764 | 43236 | 8: 69156564 | 43237 | 8: 69156580 | 43239 | 8: 69252951 | 43241 | 8: 69325956 |
| 43242 | 8: 69421607 | 43258 | 8: 69882196 | 43261 | 8: 69947806 | 43262 | 8: 69974129 | 43263 | 8: 70014064 |
| 43264 | 8: 70029487 | 43265 | 8: 70039617 | 43280 | 8: 70405010 | 43286 | 8: 70466364 | 43288 | 8: 70482064 |
| 43289 | 8: 70489717 | 43290 | 8: 70492869 | 43292 | 8: 70501163 | 43297 | 8: 70592634 | 43303 | 8: 70815570 |
| 43306 | 8: 70893052 | 43310 | 8: 70958608 | 43319 | 8: 71238004 | 43336 | 8: 71880675 | 43360 | 8: 72468833 |
| 43361 | 8: 72468916 | 43362 | 8: 72515403 | 43364 | 8: 72551579 | 43366 | 8: 72558376 | 43367 | 8: 72561704 |
| 43372 | 8: 72825901 | 43373 | 8: 72828377 | 43375 | 8: 72837275 | 43376 | 8: 72844608 | 43377 | 8: 72845970 |
| 43378 | 8: 72904078 | 43380 | 8: 72930313 | 43404 | 8: 73494061 | 43406 | 8: 73515301 | 43412 | 8: 73656442 |
| 43423 | 8: 73915262 | 43432 | 8: 74168959 | 43436 | 8: 74325878 | 43440 | 8: 74465417 | 43444 | 8: 74553402 |
| 43445 | 8: 74558074 | 43446 | 8: 74558704 | 43452 | 8: 74712678 | 43458 | 8: 74817911 | 43462 | 8: 74863602 |
| 43463 | 8: 74883099 | 43467 | 8: 75123238 | 43471 | 8: 75268745 | 43477 | 8: 75477646 | 43481 | 8: 75562796 |
| 43484 | 8: 75700129 | 43487 | 8: 75740079 | 43490 | 8: 75853292 | 43497 | 8: 76175098 | 43507 | 8: 76433813 |
| 43508 | 8: 76434785 | 43509 | 8: 76434815 | 43511 | 8: 76464537 | 43513 | 8: 76492709 | 43515 | 8: 76515741 |
| 43521 | 8: 76664342 | 43522 | 8: 76681835 | 43523 | 8: 76684305 | 43524 | 8: 76687450 | 43525 | 8: 76703859 |
| 43526 | 8: 76709352 | 43529 | 8: 76742010 | 43530 | 8: 76752971 | 43531 | 8: 76754631 | 43532 | 8: 76760562 |
| 43533 | 8: 76762856 | 43534 | 8: 76764812 | 43535 | 8: 76768935 | 43538 | 8: 76828964 | 43539 | 8: 76835585 |
| 43540 | 8: 76839168 | 43541 | 8: 76841937 | 43542 | 8: 76843765 | 43543 | 8: 76844513 | 43544 | 8: 76846849 |
| 43545 | 8: 76850281 | 43546 | 8: 76854581 | 43547 | 8: 76870958 | 43548 | 8: 76873029 | 43551 | 8: 76975704 |
| 43553 | 8: 76993039 | 43554 | 8: 77006329 | 43556 | 8: 77136545 | 43564 | 8: 77562434 | 43581 | 8: 78166070 |
| 43589 | 8: 78358551 | 43595 | 8: 78556780 | 43616 | 8: 79447931 | 43642 | 8: 80268872 | 43648 | 8: 80405770 |
| 43652 | 8: 80565130 | 43656 | 8: 80613713 | 43660 | 8: 80836273 | 43669 | 8: 80974113 | 43686 | 8: 81278385 |
| 43692 | 8: 81389738 | 43695 | 8: 81454715 | 43696 | 8: 81454750 | 43698 | 8: 81487284 | 43699 | 8: 81487295 |
| 43704 | 8: 81616321 | 43712 | 8: 81744964 | 43716 | 8: 81860516 | 43727 | 8: 82113238 | 43728 | 8: 82116405 |
| 43730 | 8: 82160329 | 43751 | 8: 82676827 | 43752 | 8: 82677581 | 43753 | 8: 82677988 | 43759 | 8: 82823172 |
| 43762 | 8: 82865703 | 43769 | 8: 83085781 | 43782 | 8: 83515046 | 43790 | 8: 83738651 | 43794 | 8: 83793151 |
| 43795 | 8: 83793154 | 43796 | 8: 83794591 | 43797 | 8: 83794760 | 43799 | 8: 83842360 | 43803 | 8: 83879316 |
| 43808 | 8: 83976437 | 43809 | 8: 84045184 | 43811 | 8: 84100704 | 43812 | 8: 84123211 | 43813 | 8: 84134245 |
| 43815 | 8: 84172945 | 43829 | 8: 84494416 | 43835 | 8: 84811346 | 43842 | 8: 84912804 | 43858 | 8: 85366261 |
| 43873 | 8: 85725330 | 43875 | 8: 85804392 | 43884 | 8: 85987078 | 43888 | 8: 86079018 | 43891 | 8: 86115895 |
| 43892 | 8: 86133024 | 43895 | 8: 86219410 | 43896 | 8: 86226370 | 43905 | 8: 86537816 | 43912 | 8: 86719772 |
| 43914 | 8: 86756176 | 43916 | 8: 86779557 | 43917 | 8: 86793653 | 43920 | 8: 86935675 | 43921 | 8: 86961975 |
| 43949 | 8: 87523043 | 43955 | 8: 87701857 | 43967 | 8: 88033337 | 43968 | 8: 88126576 | 43977 | 8: 88363375 |
| 43986 | 8: 88562919 | 43987 | 8: 88572133 | 43995 | 8: 88712829 | 43997 | 8: 88762685 | 44001 | 8: 88829394 |
| 44007 | 8: 88990195 | 44013 | 8: 89098414 | 44014 | 8: 89113184 | 44016 | 8: 89140453 | 44018 | 8: 89141946 |
| 44019 | 8: 89141971 | 44020 | 8: 89142000 | 44021 | 8: 89142063 | 44024 | 8: 89269473 | 44033 | 8: 89533107 |
| 44034 | 8: 89533116 | 44035 | 8: 89533121 | 44037 | 8: 89557638 | 44041 | 8: 89571402 | 44046 | 8: 89722190 |
| 44049 | 8: 89770693 | 44056 | 8: 89926318 | 44059 | 8: 90070089 | 44062 | 8: 90193893 | 44084 | 8: 90853473 |
| 44085 | 8: 90853557 | 44086 | 8: 90853588 | 44087 | 8: 90853599 | 44101 | 8: 91152034 | 44102 | 8: 91161810 |
| 44106 | 8: 91289222 | 44110 | 8: 91381933 | 44111 | 8: 91382574 | 44129 | 8: 91948504 | 44137 | 8: 91989858 |
| 44138 | 8: 91995185 | 44143 | 8: 92008855 | 44149 | 8: 92136613 | 44168 | 8: 92747513 | 44178 | 8: 93068614 |
| 44179 | 8: 93068725 | 44204 | 8: 93841029 | 44208 | 8: 94082045 | 44212 | 8: 94260204 | 44220 | 8: 94620469 |
| 44221 | 8: 94620534 | 44222 | 8: 94621015 | 44223 | 8: 94623988 | 44224 | 8: 94624900 | 44225 | 8: 94672379 |
| 44226 | 8: 94690978 | 44232 | 8: 94939320 | 44241 | 8: 95337173 | 44259 | 8: 95796796 | 44263 | 8: 95951526 |
| 44264 | 8: 95977921 | 44266 | 8: 96012156 | 44272 | 8: 96165982 | 44273 | 8: 96193360 | 44281 | 8: 96384779 |
| 44282 | 8: 96445578 | 44289 | 8: 96573262 | 44308 | 8: 97157793 | 44317 | 8: 97299078 | 44319 | 8: 97368811 |
| 44326 | 8: 97495202 | 44327 | 8: 97557161 | 44329 | 8: 97577094 | 44330 | 8: 97601680 | 44335 | 8: 97795768 |
| 44350 | 8: 98245729 | 44360 | 8: 98592583 | 44362 | 8: 98625126 | 44367 | 8: 98741152 | 44368 | 8: 98785042 |
| 44370 | 8: 98829420 | 44375 | 8: 98919680 | 44386 | 8: 99142994 | 44392 | 8: 99207386 | 44397 | 8: 99291886 |
| 44400 | 8: 99333533 | 44404 | 8: 99388416 | 44405 | 8: 99406177 | 44407 | 8: 99412670 | 44409 | 8: 99476683 |
| 44413 | 8: 99525142 | 44415 | 8: 99551509 | 44425 | 8: 99654305 | 44429 | 8: 99703737 | 44435 | 8: 99959755 |
| 44436 | 8: 99982505 | 44437 | 8: 100010762 | 44446 | 8: 100374163 | 44448 | 8: 100401670 | 44449 | 8: 100409615 |
| 44453 | 8: 100428898 | 44455 | 8: 100470224 | 44466 | 8: 100654637 | 44471 | 8: 100832272 | 44472 | 8: 100876059 |
| 44478 | 8: 101026237 | 44482 | 8: 101247151 | 44485 | 8: 101301768 | 44486 | 8: 101312150 | 44494 | 8: 101441924 |
| 44496 | 8: 101485459 | 44500 | 8: 101643451 | 44502 | 8: 101701776 | 44506 | 8: 101779830 | 44507 | 8: 101800844 |
| 44517 | 8: 102113941 | 44523 | 8: 102234774 | 44525 | 8: 102290809 | 44526 | 8: 102291109 | 44527 | 8: 102291157 |
| 44528 | 8: 102291228 | 44529 | 8: 102291230 | 44530 | 8: 102291256 | 44531 | 8: 102291263 | 44532 | 8: 102293933 |
| 44533 | 8: 102293974 | 44534 | 8: 102299598 | 44535 | 8: 102299601 | 44536 | 8: 102305536 | 44538 | 8: 102377736 |
| 44540 | 8: 102402762 | 44541 | 8: 102408863 | 44542 | 8: 102411405 | 44546 | 8: 102434252 | 44554 | 8: 102594527 |
| 44555 | 8: 102594773 | 44556 | 8: 102595590 | 44557 | 8: 102617174 | 44558 | 8: 102618622 | 44560 | 8: 102626997 |
| 44561 | 8: 102634985 | 44562 | 8: 102640018 | 44564 | 8: 102642339 | 44566 | 8: 102645680 | 44567 | 8: 102648250 |
| 44568 | 8: 102648741 | 44577 | 8: 102834472 | 44579 | 8: 102889304 | 44583 | 8: 102931846 | 44596 | 8: 103307658 |
| 44597 | 8: 103316620 | 44598 | 8: 103323065 | 44599 | 8: 103323527 | 44600 | 8: 103415237 | 44601 | 8: 103442278 |
| 44602 | 8: 103449094 | 44604 | 8: 103484976 | 44605 | 8: 103489848 | 44606 | 8: 103500362 | 44607 | 8: 103516850 |
| 44608 | 8: 103529815 | 44609 | 8: 103560463 | 44610 | 8: 103572704 | 44611 | 8: 103575282 | 44612 | 8: 103580891 |
| 44613 | 8: 103584228 | 44614 | 8: 103588272 | 44615 | 8: 103589059 | 44616 | 8: 103591535 | 44618 | 8: 103596979 |
| 44619 | 8: 103598675 | 44620 | 8: 103603170 | 44621 | 8: 103612096 | 44622 | 8: 103612794 | 44624 | 8: 103623951 |
| 44625 | 8: 103634947 | 44626 | 8: 103640204 | 44627 | 8: 103645354 | 44628 | 8: 103649615 | 44630 | 8: 103673828 |
| 44634 | 8: 103783688 | 44646 | 8: 104113096 | 44647 | 8: 104114568 | 44650 | 8: 104204807 | 44654 | 8: 104251576 |
| 44655 | 8: 104271837 | 44658 | 8: 104292759 | 44659 | 8: 104322730 | 44661 | 8: 104414493 | 44663 | 8: 104420746 |
| 44669 | 8: 104546178 | 44674 | 8: 104630353 | 44679 | 8: 104760159 | 44681 | 8: 104815425 | 44694 | 8: 105160429 |
| 44716 | 8: 105525844 | 44719 | 8: 105592649 | 44722 | 8: 105630176 | 44725 | 8: 105696501 | 44727 | 8: 105765759 |
| 44728 | 8: 105774110 | 44732 | 8: 105846896 | 44739 | 8: 106082143 | 44744 | 8: 106427938 | 44759 | 8: 106936156 |
| 44760 | 8: 106961448 | 44761 | 8: 106981761 | 44762 | 8: 107001526 | 44808 | 8: 108127783 | 44829 | 8: 108978247 |
| 44850 | 8: 109853668 | 44855 | 8: 110002325 | 44857 | 8: 110059433 | 44860 | 8: 110157715 | 44862 | 8: 110244165 |
| 44863 | 8: 110244337 | 44865 | 8: 110248495 | 44866 | 8: 110251397 | 44870 | 8: 110445148 | 44871 | 8: 110445202 |
| 44872 | 8: 110445222 | 44874 | 8: 110540868 | 44880 | 8: 110749028 | 44882 | 8: 110764849 | 44888 | 8: 110972844 |
| 44890 | 8: 110979223 | 44892 | 8: 111002543 | 44893 | 8: 111081452 | 44897 | 8: 111284112 | 44932 | 9: 349617 |
| 44935 | 9: 449265 | 44965 | 9: 761293 | 44966 | 9: 761398 | 44970 | 9: 946489 | 44983 | 9: 1228475 |
| 44984 | 9: 1228520 | 44985 | 9: 1228587 | 44986 | 9: 1228611 | 44987 | 9: 1228669 | 44997 | 9: 1530507 |
| 44998 | 9: 1530528 | 45013 | 9: 1926540 | 45016 | 9: 2022731 | 45017 | 9: 2022740 | 45084 | 9: 4060756 |
| 45090 | 9: 4309847 | 45101 | 9: 4645222 | 45115 | 9: 5086559 | 45159 | 9: 6390476 | 45161 | 9: 6450647 |
| 45163 | 9: 6559884 | 45174 | 9: 6887577 | 45189 | 9: 7542350 | 45206 | 9: 7865211 | 45219 | 9: 8218235 |
| 45220 | 9: 8218462 | 45223 | 9: 8257979 | 45224 | 9: 8258011 | 45225 | 9: 8258047 | 45241 | 9: 8662565 |
| 45242 | 9: 8691938 | 45245 | 9: 8740852 | 45246 | 9: 8740922 | 45247 | 9: 8741004 | 45255 | 9: 9002672 |
| 45259 | 9: 9073133 | 45265 | 9: 9212931 | 45269 | 9: 9363423 | 45281 | 9: 9866377 | 45288 | 9: 10026119 |
| 45290 | 9: 10179658 | 45293 | 9: 10229582 | 45295 | 9: 10270107 | 45302 | 9: 10376631 | 45307 | 9: 10412914 |
| 45343 | 9: 10650283 | 45377 | 9: 10813074 | 45393 | 9: 11379384 | 45398 | 9: 11488885 | 45413 | 9: 11816817 |
| 45418 | 9: 11933227 | 45428 | 9: 12091110 | 45431 | 9: 12153363 | 45433 | 9: 12204845 | 45435 | 9: 12250871 |
| 45445 | 9: 12499635 | 45446 | 9: 12509673 | 45452 | 9: 12645170 | 45471 | 9: 12766564 | 45481 | 9: 13100266 |
| 45485 | 9: 13105495 | 45491 | 9: 13132484 | 45496 | 9: 13287682 | 45498 | 9: 13361022 | 45503 | 9: 13494908 |
| 45509 | 9: 13684115 | 45510 | 9: 13698663 | 45517 | 9: 13880641 | 45520 | 9: 13989518 | 45523 | 9: 14095633 |
| 45524 | 9: 14124920 | 45527 | 9: 14203195 | 45540 | 9: 14468437 | 45545 | 9: 14564006 | 45548 | 9: 14665440 |
| 45550 | 9: 14717693 | 45564 | 9: 15013572 | 45568 | 9: 15038319 | 45570 | 9: 15071350 | 45571 | 9: 15081586 |
| 45574 | 9: 15123580 | 45577 | 9: 15163893 | 45578 | 9: 15175648 | 45585 | 9: 15369931 | 45587 | 9: 15406354 |
| 45595 | 9: 15497571 | 45596 | 9: 15499363 | 45597 | 9: 15499412 | 45598 | 9: 15499535 | 45599 | 9: 15511160 |
| 45602 | 9: 15527205 | 45604 | 9: 15548110 | 45605 | 9: 15553535 | 45606 | 9: 15558085 | 45607 | 9: 15560500 |
| 45608 | 9: 15560527 | 45609 | 9: 15562142 | 45623 | 9: 15687216 | 45631 | 9: 15868930 | 45644 | 9: 16138159 |
| 45653 | 9: 16384844 | 45657 | 9: 16537269 | 45674 | 9: 17031734 | 45675 | 9: 17031771 | 45687 | 9: 17461271 |
| 45692 | 9: 17595872 | 45717 | 9: 18430308 | 45721 | 9: 18529694 | 45722 | 9: 18552564 | 45724 | 9: 18645348 |
| 45726 | 9: 18717619 | 45727 | 9: 18731789 | 45729 | 9: 18755368 | 45731 | 9: 18791756 | 45732 | 9: 18791819 |
| 45733 | 9: 18791889 | 45738 | 9: 18936509 | 45743 | 9: 19046499 | 45745 | 9: 19139676 | 45753 | 9: 19435053 |
| 45776 | 9: 20235228 | 45779 | 9: 20303916 | 45816 | 9: 21552246 | 45824 | 9: 21653355 | 45833 | 9: 21833987 |
| 45835 | 9: 21852388 | 45840 | 9: 21940817 | 45846 | 9: 22080892 | 45848 | 9: 22133199 | 45865 | 9: 22573520 |
| 45867 | 9: 22594113 | 45874 | 9: 22795139 | 45879 | 9: 22958243 | 45889 | 9: 23151477 | 45890 | 9: 23173931 |
| 45894 | 9: 23218165 | 45898 | 9: 23339249 | 45900 | 9: 23430616 | 45901 | 9: 23441779 | 45904 | 9: 23456305 |
| 45906 | 9: 23472649 | 45909 | 9: 23546774 | 45915 | 9: 23672358 | 45925 | 9: 23857068 | 45932 | 9: 24029631 |
| 45937 | 9: 24145793 | 45943 | 9: 24238183 | 45951 | 9: 24426538 | 45970 | 9: 25002484 | 45980 | 9: 25350079 |
| 45981 | 9: 25353185 | 45989 | 9: 25530018 | 45997 | 9: 25755576 | 45999 | 9: 25785154 | 46003 | 9: 25878075 |
| 46010 | 9: 26028894 | 46015 | 9: 26134547 | 46018 | 9: 26208349 | 46028 | 9: 26467397 | 46039 | 9: 26838528 |
| 46040 | 9: 26868103 | 46041 | 9: 26898919 | 46048 | 9: 27183994 | 46054 | 9: 27249330 | 46057 | 9: 27304567 |
| 46058 | 9: 27317016 | 46061 | 9: 27408248 | 46071 | 9: 27587922 | 46077 | 9: 27702582 | 46094 | 9: 28136433 |
| 46108 | 9: 28431328 | 46121 | 9: 28644696 | 46127 | 9: 28779206 | 46165 | 9: 29391430 | 46177 | 9: 29530654 |
| 46179 | 9: 29544266 | 46183 | 9: 29615614 | 46205 | 9: 30230309 | 46207 | 9: 30256998 | 46208 | 9: 30257023 |
| 46209 | 9: 30257052 | 46212 | 9: 30289673 | 46219 | 9: 30469930 | 46224 | 9: 30615123 | 46227 | 9: 30674435 |
| 46229 | 9: 30739336 | 46237 | 9: 30892065 | 46239 | 9: 30932509 | 46240 | 9: 30932519 | 46241 | 9: 30932556 |
| 46247 | 9: 31029451 | 46249 | 9: 31141266 | 46252 | 9: 31227971 | 46253 | 9: 31227973 | 46255 | 9: 31253066 |
| 46261 | 9: 31394461 | 46271 | 9: 31555978 | 46272 | 9: 31588762 | 46273 | 9: 31588794 | 46275 | 9: 31725734 |
| 46283 | 9: 31984894 | 46290 | 9: 32052617 | 46291 | 9: 32063888 | 46292 | 9: 32063963 | 46296 | 9: 32225638 |
| 46300 | 9: 32284534 | 46301 | 9: 32291884 | 46307 | 9: 32437975 | 46308 | 9: 32501496 | 46312 | 9: 32638914 |
| 46315 | 9: 32720871 | 46342 | 9: 33372243 | 46349 | 9: 33488593 | 46350 | 9: 33496695 | 46356 | 9: 33730807 |
| 46357 | 9: 33736686 | 46358 | 9: 33736719 | 46365 | 9: 33922999 | 46376 | 9: 34234529 | 46377 | 9: 34239640 |
| 46383 | 9: 34398858 | 46405 | 9: 35059112 | 46422 | 9: 35505083 | 46424 | 9: 35630351 | 46428 | 9: 35723297 |
| 46429 | 9: 35723312 | 46430 | 9: 35723316 | 46431 | 9: 35723347 | 46432 | 9: 35723412 | 46438 | 9: 35923167 |
| 46445 | 9: 36108895 | 46446 | 9: 36118700 | 46448 | 9: 36196365 | 46457 | 9: 36321163 | 46464 | 9: 36515253 |
| 46466 | 9: 36617540 | 46468 | 9: 36686271 | 46470 | 9: 36765440 | 46475 | 9: 36837540 | 46482 | 9: 36952973 |
| 46485 | 9: 36987967 | 46495 | 9: 37162141 | 46500 | 9: 37248499 | 46501 | 9: 37262739 | 46504 | 9: 37446666 |
| 46518 | 9: 37786775 | 46532 | 9: 38042996 | 46538 | 9: 38200604 | 46540 | 9: 38214307 | 46541 | 9: 38222757 |
| 46542 | 9: 38234690 | 46548 | 9: 38345134 | 46549 | 9: 38346604 | 46550 | 9: 38349255 | 46551 | 9: 38361398 |
| 46555 | 9: 38439726 | 46556 | 9: 38458789 | 46558 | 9: 38513565 | 46560 | 9: 38544090 | 46562 | 9: 38561388 |
| 46568 | 9: 38630128 | 46575 | 9: 38725892 | 46578 | 9: 38828299 | 46585 | 9: 38890408 | 46589 | 9: 38943133 |
| 46590 | 9: 38963419 | 46603 | 9: 39377711 | 46610 | 9: 39610211 | 46611 | 9: 39633208 | 46612 | 9: 39637416 |
| 46613 | 9: 39638258 | 46614 | 9: 39639117 | 46616 | 9: 39648286 | 46622 | 9: 39764057 | 46623 | 9: 39784094 |
| 46624 | 9: 39793093 | 46627 | 9: 39807347 | 46628 | 9: 39807953 | 46629 | 9: 39811555 | 46634 | 9: 39934012 |
| 46635 | 9: 39956055 | 46638 | 9: 40007646 | 46646 | 9: 40343063 | 46647 | 9: 40346754 | 46649 | 9: 40351241 |
| 46650 | 9: 40353887 | 46651 | 9: 40359510 | 46652 | 9: 40394895 | 46653 | 9: 40405455 | 46658 | 9: 40559500 |
| 46664 | 9: 40689518 | 46670 | 9: 40792949 | 46673 | 9: 40849838 | 46674 | 9: 40851485 | 46675 | 9: 40876197 |
| 46676 | 9: 40920502 | 46677 | 9: 40928750 | 46678 | 9: 40936292 | 46679 | 9: 40949947 | 46680 | 9: 40960994 |
| 46681 | 9: 40986746 | 46684 | 9: 41057481 | 46690 | 9: 41209197 | 46698 | 9: 41519871 | 46702 | 9: 41630405 |
| 46705 | 9: 41713976 | 46712 | 9: 42008068 | 46714 | 9: 42049316 | 46715 | 9: 42066260 | 46716 | 9: 42073188 |
| 46721 | 9: 42199260 | 46722 | 9: 42202691 | 46723 | 9: 42206745 | 46724 | 9: 42207524 | 46726 | 9: 42218456 |
| 46738 | 9: 42539807 | 46741 | 9: 42738988 | 46742 | 9: 42751569 | 46743 | 9: 42754079 | 46751 | 9: 43079312 |
| 46752 | 9: 43096509 | 46754 | 9: 43117290 | 46755 | 9: 43117292 | 46756 | 9: 43117313 | 46757 | 9: 43117336 |
| 46758 | 9: 43117351 | 46759 | 9: 43155466 | 46760 | 9: 43155519 | 46761 | 9: 43155650 | 46766 | 9: 43352867 |
| 46779 | 9: 43685269 | 46787 | 9: 43948226 | 46803 | 9: 44341413 | 46817 | 9: 44599779 | 46820 | 9: 44637804 |
| 46826 | 9: 44854910 | 46842 | 9: 45505888 | 46845 | 9: 45550200 | 46868 | 9: 46443428 | 46877 | 9: 46667396 |
| 46883 | 9: 46847687 | 46895 | 9: 47304057 | 46923 | 9: 48075628 | 46930 | 9: 48336145 | 46939 | 9: 48787417 |
| 46954 | 9: 49224046 | 46955 | 9: 49225376 | 46982 | 9: 49910883 | 46995 | 9: 50277864 | 47007 | 9: 50567297 |
| 47008 | 9: 50567298 | 47009 | 9: 50567390 | 47012 | 9: 50646985 | 47015 | 9: 50694324 | 47019 | 9: 50797460 |
| 47059 | 9: 52046890 | 47080 | 9: 52645687 | 47088 | 9: 52787927 | 47093 | 9: 52848517 | 47095 | 9: 52861826 |
| 47120 | 9: 53314350 | 47139 | 9: 54071333 | 47143 | 9: 54193058 | 47146 | 9: 54279421 | 47153 | 9: 54522488 |
| 47161 | 9: 54675346 | 47181 | 9: 55237255 | 47192 | 9: 55526568 | 47193 | 9: 55526660 | 47194 | 9: 55529308 |
| 47228 | 9: 56645296 | 47230 | 9: 56687693 | 47234 | 9: 56760440 | 47236 | 9: 56787195 | 47245 | 9: 57057433 |
| 47259 | 9: 57574987 | 47260 | 9: 57574995 | 47262 | 9: 57634613 | 47272 | 9: 57793857 | 47276 | 9: 57849001 |
| 47284 | 9: 58149441 | 47287 | 9: 58311080 | 47288 | 9: 58333810 | 47293 | 9: 58457036 | 47301 | 9: 58652669 |
| 47314 | 9: 59159045 | 47351 | 9: 59715217 | 47353 | 9: 59771120 | 47355 | 9: 59857183 | 47362 | 9: 60072130 |
| 47364 | 9: 60134972 | 47367 | 9: 60247427 | 47369 | 9: 60291706 | 47372 | 9: 60358510 | 47377 | 9: 60481368 |
| 47380 | 9: 60536321 | 47383 | 9: 60631920 | 47388 | 9: 60745480 | 47393 | 9: 60896389 | 47394 | 9: 60903838 |
| 47397 | 9: 60928250 | 47398 | 9: 60928272 | 47400 | 9: 60929060 | 47401 | 9: 60930496 | 47402 | 9: 60932205 |
| 47403 | 9: 60936701 | 47406 | 9: 61034023 | 47420 | 9: 61625663 | 47428 | 9: 61883689 | 47437 | 9: 62153095 |
| 47438 | 9: 62160605 | 47441 | 9: 62239934 | 47444 | 9: 62407115 | 47446 | 9: 62441325 | 47448 | 9: 62541808 |
| 47449 | 9: 62541844 | 47450 | 9: 62541925 | 47451 | 9: 62569285 | 47453 | 9: 62596000 | 47475 | 9: 63213406 |
| 47481 | 9: 63334601 | 47482 | 9: 63334709 | 47483 | 9: 63334737 | 47488 | 9: 63564229 | 47490 | 9: 63662723 |
| 47495 | 9: 63879901 | 47505 | 9: 64262650 | 47522 | 9: 64653298 | 47523 | 9: 64675425 | 47526 | 9: 64806879 |
| 47527 | 9: 64818979 | 47536 | 9: 65014222 | 47537 | 9: 65014886 | 47538 | 9: 65015900 | 47539 | 9: 65016625 |
| 47540 | 9: 65017299 | 47541 | 9: 65017970 | 47542 | 9: 65018705 | 47543 | 9: 65020386 | 47544 | 9: 65022012 |
| 47545 | 9: 65022960 | 47546 | 9: 65024531 | 47547 | 9: 65025971 | 47548 | 9: 65026593 | 47549 | 9: 65027537 |
| 47550 | 9: 65029266 | 47551 | 9: 65031641 | 47552 | 9: 65033856 | 47553 | 9: 65034695 | 47554 | 9: 65037061 |
| 47555 | 9: 65038847 | 47556 | 9: 65039895 | 47557 | 9: 65041060 | 47558 | 9: 65041925 | 47559 | 9: 65046945 |
| 47560 | 9: 65048753 | 47561 | 9: 65051331 | 47562 | 9: 65052345 | 47564 | 9: 65062111 | 47565 | 9: 65063669 |
| 47566 | 9: 65065713 | 47567 | 9: 65069167 | 47568 | 9: 65072452 | 47569 | 9: 65074602 | 47570 | 9: 65075729 |
| 47571 | 9: 65076561 | 47572 | 9: 65077195 | 47573 | 9: 65077758 | 47574 | 9: 65078630 | 47575 | 9: 65118677 |
| 47576 | 9: 65123323 | 47578 | 9: 65179118 | 47579 | 9: 65235666 | 47580 | 9: 65243193 | 47581 | 9: 65244259 |
| 47586 | 9: 65383024 | 47589 | 9: 65443065 | 47590 | 9: 65446545 | 47591 | 9: 65478206 | 47592 | 9: 65483403 |
| 47598 | 9: 65568211 | 47599 | 9: 65570457 | 47600 | 9: 65572272 | 47602 | 9: 65604455 | 47609 | 9: 65891235 |
| 47612 | 9: 65971970 | 47615 | 9: 66011528 | 47638 | 9: 66597468 | 47648 | 9: 66989120 | 47657 | 9: 67225760 |
| 47663 | 9: 67332887 | 47668 | 9: 67459980 | 47681 | 9: 67796936 | 47682 | 9: 67823147 | 47686 | 9: 67954748 |
| 47705 | 9: 68370513 | 47708 | 9: 68427995 | 47720 | 9: 68912666 | 47721 | 9: 68917528 | 47729 | 9: 69060023 |
| 47730 | 9: 69093647 | 47731 | 9: 69104702 | 47732 | 9: 69114502 | 47738 | 9: 69297159 | 47741 | 9: 69407676 |
| 47750 | 9: 69583761 | 47751 | 9: 69599853 | 47761 | 9: 69896845 | 47767 | 9: 70012144 | 47777 | 9: 70295013 |
| 47779 | 9: 70342326 | 47780 | 9: 70347483 | 47786 | 9: 70409386 | 47787 | 9: 70409422 | 47789 | 9: 70429923 |
| 47797 | 9: 70751662 | 47798 | 9: 70753720 | 47805 | 9: 70887910 | 47809 | 9: 71049805 | 47811 | 9: 71060333 |
| 47813 | 9: 71107122 | 47815 | 9: 71116611 | 47820 | 9: 71251152 | 47822 | 9: 71292789 | 47824 | 9: 71344053 |
| 47825 | 9: 71356859 | 47826 | 9: 71384334 | 47828 | 9: 71407368 | 47829 | 9: 71445600 | 47831 | 9: 71492315 |
| 47834 | 9: 71512303 | 47835 | 9: 71513737 | 47840 | 9: 71612772 | 47844 | 9: 71651985 | 47848 | 9: 71736589 |
| 47849 | 9: 71761625 | 47864 | 9: 72304039 | 47867 | 9: 72384960 | 47877 | 9: 72575284 | 47882 | 9: 72722658 |
| 47883 | 9: 72725124 | 47891 | 9: 72888193 | 47892 | 9: 72973276 | 47893 | 9: 72989037 | 47895 | 9: 73138567 |
| 47897 | 9: 73175040 | 47902 | 9: 73223596 | 47903 | 9: 73244841 | 47915 | 9: 73594271 | 47920 | 9: 73701336 |
| 47921 | 9: 73701377 | 47937 | 9: 74031324 | 47939 | 9: 74043724 | 47948 | 9: 74315428 | 47955 | 9: 74575135 |
| 47957 | 9: 74617923 | 47959 | 9: 74622543 | 47974 | 9: 75094490 | 47975 | 9: 75114725 | 47979 | 9: 75261135 |
| 47984 | 9: 75482552 | 47987 | 9: 75505924 | 47991 | 9: 75564777 | 47995 | 9: 75627446 | 47999 | 9: 75748546 |
| 48000 | 9: 75749661 | 48001 | 9: 75754806 | 48002 | 9: 75754987 | 48014 | 9: 76061643 | 48016 | 9: 76103605 |
| 48022 | 9: 76314846 | 48023 | 9: 76319841 | 48038 | 9: 76900400 | 48041 | 9: 76954502 | 48042 | 9: 76961448 |
| 48045 | 9: 76972318 | 48048 | 9: 77037622 | 48049 | 9: 77047736 | 48050 | 9: 77049274 | 48058 | 9: 77255407 |
| 48063 | 9: 77401724 | 48071 | 9: 77497521 | 48150 | 9: 78018701 | 48157 | 9: 78383301 | 48163 | 9: 78618960 |
| 48166 | 9: 78653185 | 48173 | 9: 78856788 | 48176 | 9: 78984268 | 48192 | 9: 79629564 | 48204 | 9: 79900857 |
| 48206 | 9: 79927447 | 48209 | 9: 79992577 | 48211 | 9: 79999363 | 48220 | 9: 80143039 | 48222 | 9: 80223447 |
| 48226 | 9: 80281892 | 48235 | 9: 80379126 | 48251 | 9: 80913419 | 48252 | 9: 80926042 | 48253 | 9: 80927048 |
| 48254 | 9: 80927101 | 48255 | 9: 80935101 | 48256 | 9: 80938115 | 48260 | 9: 80960455 | 48281 | 9: 81389583 |
| 48286 | 9: 81502463 | 48289 | 9: 81518292 | 48299 | 9: 81745339 | 48304 | 9: 81841021 | 48317 | 9: 82097477 |
| 48318 | 9: 82097512 | 48319 | 9: 82100313 | 48337 | 9: 82534749 | 48362 | 9: 82854874 | 48364 | 9: 82923714 |
| 48369 | 9: 83119086 | 48374 | 9: 83237503 | 48377 | 9: 83309469 | 48391 | 9: 83641203 | 48392 | 9: 83672779 |
| 48399 | 9: 83841026 | 48400 | 9: 83857767 | 48418 | 9: 84276446 | 48447 | 9: 85144864 | 48452 | 9: 85201593 |
| 48455 | 9: 85222343 | 48457 | 9: 85277103 | 48474 | 9: 85565902 | 48476 | 9: 85582831 | 48477 | 9: 85582845 |
| 48478 | 9: 85582856 | 48479 | 9: 85582930 | 48485 | 9: 85757204 | 48486 | 9: 85777430 | 48487 | 9: 85777435 |
| 48488 | 9: 85777468 | 48489 | 9: 85777555 | 48492 | 9: 85801137 | 48494 | 9: 85812059 | 48500 | 9: 85926846 |
| 48507 | 9: 86110491 | 48514 | 9: 86329047 | 48532 | 9: 86823737 | 48534 | 9: 86935158 | 48537 | 9: 86963116 |
| 48539 | 9: 86989548 | 48541 | 9: 87028872 | 48556 | 9: 87348985 | 48561 | 9: 87384465 | 48568 | 9: 87494785 |
| 48570 | 9: 87503445 | 48571 | 9: 87504577 | 48572 | 9: 87542588 | 48573 | 9: 87544854 | 48593 | 9: 87878916 |
| 48595 | 9: 87890519 | 48596 | 9: 87917511 | 48598 | 9: 87929924 | 48599 | 9: 87952953 | 48600 | 9: 87962089 |
| 48601 | 9: 87962734 | 48603 | 9: 87966017 | 48604 | 9: 87966917 | 48605 | 9: 87975738 | 48610 | 9: 88087724 |
| 48612 | 9: 88140632 | 48613 | 9: 88160188 | 48626 | 9: 88569166 | 48628 | 9: 88636603 | 48629 | 9: 88636618 |
| 48630 | 9: 88636634 | 48635 | 9: 88732430 | 48644 | 9: 88925002 | 48646 | 9: 88943609 | 48654 | 9: 89094468 |
| 48656 | 9: 89166354 | 48658 | 9: 89189099 | 48666 | 9: 89326613 | 48667 | 9: 89345202 | 48668 | 9: 89346130 |
| 48670 | 9: 89356829 | 48671 | 9: 89373227 | 48672 | 9: 89380245 | 48673 | 9: 89387102 | 48674 | 9: 89399914 |
| 48676 | 9: 89410907 | 48677 | 9: 89417136 | 48678 | 9: 89423178 | 48680 | 9: 89436296 | 48681 | 9: 89443866 |
| 48682 | 9: 89445782 | 48683 | 9: 89445926 | 48684 | 9: 89447805 | 48685 | 9: 89454999 | 48688 | 9: 89471837 |
| 48690 | 9: 89496573 | 48698 | 9: 89664367 | 48699 | 9: 89708244 | 48700 | 9: 89708807 | 48709 | 9: 89919288 |
| 48733 | 9: 90540018 | 48744 | 9: 90795244 | 48745 | 9: 90796860 | 48750 | 9: 90844111 | 48751 | 9: 90845595 |
| 48752 | 9: 90845718 | 48762 | 9: 91108815 | 48767 | 9: 91144245 | 48776 | 9: 91423091 | 48783 | 9: 91591725 |
| 48784 | 9: 91611257 | 48786 | 9: 91670541 | 48789 | 9: 91728675 | 48790 | 9: 91734111 | 48794 | 9: 91799932 |
| 48795 | 9: 91808084 | 48802 | 9: 92017600 | 48804 | 9: 92051007 | 48808 | 9: 92146096 | 48809 | 9: 92173136 |
| 48823 | 9: 92434720 | 48827 | 9: 92618757 | 48831 | 9: 92719541 | 48833 | 9: 92726807 | 48834 | 9: 92726908 |
| 48835 | 9: 92732978 | 48844 | 9: 92935922 | 48848 | 9: 93101440 | 48860 | 9: 93255745 | 48864 | 9: 93329746 |
| 48865 | 9: 93335995 | 48868 | 9: 93434039 | 48869 | 9: 93439818 | 48871 | 9: 93544670 | 48872 | 9: 93562913 |
| 48873 | 9: 93583179 | 48874 | 9: 93589365 | 48876 | 9: 93593753 | 48878 | 9: 93648481 | 48881 | 9: 93756818 |
| 48882 | 9: 93761851 | 48888 | 9: 93864628 | 48899 | 9: 94037282 | 48902 | 9: 94059439 | 48903 | 9: 94075786 |
| 48908 | 9: 94239134 | 48909 | 9: 94242901 | 48916 | 9: 94279436 | 48925 | 9: 94506574 | 48926 | 9: 94511166 |
| 48928 | 9: 94549002 | 48929 | 9: 94552504 | 48930 | 9: 94571641 | 48938 | 9: 94770144 | 48943 | 9: 94849176 |
| 48951 | 9: 95011385 | 48952 | 9: 95011440 | 48954 | 9: 95075297 | 48957 | 9: 95117095 | 48958 | 9: 95119909 |
| 48959 | 9: 95120912 | 48960 | 9: 95120918 | 48961 | 9: 95121102 | 48963 | 9: 95137543 | 48965 | 9: 95146949 |
| 48966 | 9: 95149713 | 48967 | 9: 95151479 | 48968 | 9: 95151880 | 48969 | 9: 95154305 | 48970 | 9: 95159375 |
| 48971 | 9: 95171473 | 48972 | 9: 95239467 | 48975 | 9: 95257977 | 48996 | 9: 95960164 | 48997 | 9: 95961292 |
| 48998 | 9: 95964766 | 48999 | 9: 95965222 | 49013 | 9: 96262623 | 49016 | 9: 96335077 | 49018 | 9: 96352203 |
| 49020 | 9: 96364316 | 49021 | 9: 96365825 | 49023 | 9: 96394133 | 49026 | 9: 96454587 | 49027 | 9: 96459992 |
| 49028 | 9: 96460776 | 49029 | 9: 96462068 | 49030 | 9: 96464377 | 49031 | 9: 96464974 | 49032 | 9: 96465729 |
| 49033 | 9: 96467828 | 49034 | 9: 96471984 | 49035 | 9: 96474365 | 49036 | 9: 96475139 | 49037 | 9: 96477053 |
| 49038 | 9: 96478790 | 49039 | 9: 96480951 | 49040 | 9: 96482190 | 49041 | 9: 96487368 | 49045 | 9: 96575319 |
| 49047 | 9: 96602946 | 49050 | 9: 96636351 | 49064 | 9: 96811911 | 49067 | 9: 96862976 | 49068 | 9: 96865435 |
| 49071 | 9: 96875852 | 49072 | 9: 96886282 | 49078 | 9: 97002454 | 49079 | 9: 97005408 | 49080 | 9: 97006623 |
| 49107 | 9: 97443586 | 49108 | 9: 97443633 | 49112 | 9: 97554130 | 49114 | 9: 97571949 | 49117 | 9: 97636004 |
| 49122 | 9: 97688394 | 49125 | 9: 97734208 | 49128 | 9: 97781409 | 49129 | 9: 97814093 | 49132 | 9: 97896936 |
| 49140 | 9: 97952202 | 49142 | 9: 97987774 | 49146 | 9: 98072257 | 49147 | 9: 98090254 | 49149 | 9: 98186273 |
| 49152 | 9: 98271044 | 49162 | 9: 98520046 | 49169 | 9: 98643289 | 49173 | 9: 98706036 | 49174 | 9: 98706126 |
| 49177 | 9: 98800653 | 49185 | 9: 99049511 | 49197 | 9: 99258284 | 49201 | 9: 99320226 | 49202 | 9: 99340641 |
| 49203 | 9: 99363193 | 49205 | 9: 99375752 | 49213 | 9: 99564669 | 49223 | 9: 99707846 | 49233 | 9: 99913842 |
| 49238 | 9: 100020368 | 49239 | 9: 100037134 | 49248 | 9: 100222700 | 49254 | 9: 100288205 | 49264 | 9: 100469286 |
| 49278 | 9: 100659300 | 49294 | 9: 101011242 | 49295 | 9: 101027872 | 49296 | 9: 101037743 | 49299 | 9: 101107041 |
| 49302 | 9: 101154740 | 49305 | 9: 101206979 | 49311 | 9: 101309902 | 49313 | 9: 101329946 | 49314 | 9: 101388778 |
| 49315 | 9: 101404988 | 49320 | 9: 101473236 | 49325 | 9: 101520399 | 49330 | 9: 101680647 | 49341 | 9: 101799308 |
| 49351 | 9: 101941022 | 49353 | 9: 101969310 | 49354 | 9: 101969389 | 49355 | 9: 101970659 | 49356 | 9: 101970691 |
| 49357 | 9: 101971384 | 49358 | 9: 101971424 | 49359 | 9: 101976339 | 49360 | 9: 101977590 | 49361 | 9: 101977632 |
| 49362 | 9: 101977719 | 49363 | 9: 101977742 | 49364 | 9: 101984019 | 49365 | 9: 101984050 | 49366 | 9: 101984250 |
| 49367 | 9: 102004205 | 49368 | 9: 102034495 | 49369 | 9: 102034523 | 49370 | 9: 102034580 | 49371 | 9: 102034582 |
| 49373 | 9: 102053353 | 49375 | 9: 102065589 | 49376 | 9: 102121235 | 49377 | 9: 102127863 | 49380 | 9: 102237624 |
| 49390 | 9: 102348030 | 49391 | 9: 102364770 | 49400 | 9: 102589137 | 49402 | 9: 102618152 | 49403 | 9: 102619914 |
| 49404 | 9: 102620509 | 49405 | 9: 102622882 | 49409 | 9: 102654533 | 49414 | 9: 102687424 | 49416 | 9: 102706741 |
| 49417 | 9: 102710336 | 49422 | 9: 102795356 | 49426 | 9: 102898617 | 49427 | 9: 102898674 | 49438 | 9: 103153314 |
| 49439 | 9: 103215814 | 49446 | 9: 103329162 | 49447 | 9: 103333336 | 49448 | 9: 103336781 | 49457 | 9: 103493999 |
| 49459 | 9: 103575109 | 49460 | 9: 103582734 | 49473 | 9: 103926483 | 49474 | 9: 103926928 | 49475 | 9: 103927077 |
| 49476 | 9: 103969118 | 49477 | 9: 104003359 | 49485 | 9: 104113993 | 49489 | 9: 104355196 | 49490 | 9: 104355303 |
| 49491 | 9: 104356588 | 49492 | 9: 104356685 | 49494 | 9: 104376710 | 49501 | 10: 84875 | 49519 | 10: 695399 |
| 49526 | 10: 769669 | 49544 | 10: 1257872 | 49554 | 10: 1412883 | 49570 | 10: 1805121 | 49574 | 10: 1814057 |
| 49576 | 10: 1856119 | 49578 | 10: 1881286 | 49579 | 10: 1881323 | 49593 | 10: 2147400 | 49602 | 10: 2351254 |
| 49605 | 10: 2373566 | 49626 | 10: 2903794 | 49627 | 10: 2903827 | 49629 | 10: 2929612 | 49630 | 10: 2932512 |
| 49647 | 10: 3209297 | 49674 | 10: 3744198 | 49675 | 10: 3744214 | 49677 | 10: 3772515 | 49678 | 10: 3774094 |
| 49679 | 10: 3802315 | 49691 | 10: 4074788 | 49692 | 10: 4104638 | 49705 | 10: 4448386 | 49707 | 10: 4468373 |
| 49709 | 10: 4484450 | 49711 | 10: 4554748 | 49716 | 10: 4604295 | 49720 | 10: 4638128 | 49726 | 10: 4828321 |
| 49727 | 10: 4829589 | 49732 | 10: 5012847 | 49735 | 10: 5111561 | 49755 | 10: 5476055 | 49758 | 10: 5506479 |
| 49775 | 10: 5547565 | 49776 | 10: 5550036 | 49777 | 10: 5557416 | 49781 | 10: 5609083 | 49797 | 10: 6019241 |
| 49799 | 10: 6059501 | 49802 | 10: 6133685 | 49812 | 10: 6396765 | 49816 | 10: 6454997 | 49818 | 10: 6473524 |
| 49820 | 10: 6505409 | 49823 | 10: 6590721 | 49826 | 10: 6677707 | 49857 | 10: 7516833 | 49859 | 10: 7543493 |
| 49862 | 10: 7600499 | 49873 | 10: 7761962 | 49880 | 10: 7901116 | 49884 | 10: 8008379 | 49898 | 10: 8326560 |
| 49905 | 10: 8450920 | 49907 | 10: 8478523 | 49911 | 10: 8510644 | 49912 | 10: 8510646 | 49914 | 10: 8553549 |
| 49915 | 10: 8553909 | 49920 | 10: 8708761 | 49922 | 10: 8777935 | 49926 | 10: 8877008 | 49931 | 10: 9028658 |
| 49934 | 10: 9110701 | 49955 | 10: 9671703 | 49956 | 10: 9680242 | 49958 | 10: 9697568 | 49963 | 10: 9786271 |
| 49968 | 10: 9804614 | 49979 | 10: 10147987 | 49980 | 10: 10172215 | 49983 | 10: 10207865 | 49985 | 10: 10220078 |
| 49990 | 10: 10286771 | 49992 | 10: 10393696 | 49993 | 10: 10400749 | 49997 | 10: 10519536 | 49999 | 10: 10536820 |
| 50000 | 10: 10537343 | 50001 | 10: 10611527 | 50002 | 10: 10633817 | 50005 | 10: 10700036 | 50006 | 10: 10706615 |
| 50007 | 10: 10740773 | 50008 | 10: 10740870 | 50009 | 10: 10740872 | 50010 | 10: 10742093 | 50012 | 10: 10745459 |
| 50020 | 10: 10855774 | 50025 | 10: 10952491 | 50026 | 10: 10983147 | 50028 | 10: 10992296 | 50031 | 10: 11037910 |
| 50037 | 10: 11124874 | 50041 | 10: 11194365 | 50042 | 10: 11199580 | 50043 | 10: 11214627 | 50044 | 10: 11214653 |
| 50051 | 10: 11459762 | 50052 | 10: 11480188 | 50053 | 10: 11484963 | 50054 | 10: 11485435 | 50055 | 10: 11533676 |
| 50062 | 10: 11746501 | 50071 | 10: 11984525 | 50072 | 10: 11993432 | 50075 | 10: 12037788 | 50077 | 10: 12049463 |
| 50078 | 10: 12049470 | 50080 | 10: 12056720 | 50081 | 10: 12058675 | 50082 | 10: 12060333 | 50085 | 10: 12193255 |
| 50087 | 10: 12210976 | 50092 | 10: 12363067 | 50096 | 10: 12446219 | 50104 | 10: 12563184 | 50108 | 10: 12662259 |
| 50112 | 10: 12776928 | 50118 | 10: 13028042 | 50125 | 10: 13148280 | 50129 | 10: 13195695 | 50130 | 10: 13201885 |
| 50137 | 10: 13233050 | 50141 | 10: 13251806 | 50146 | 10: 13290512 | 50155 | 10: 13433297 | 50159 | 10: 13500606 |
| 50160 | 10: 13521494 | 50163 | 10: 13603249 | 50164 | 10: 13604834 | 50184 | 10: 13904093 | 50186 | 10: 13947394 |
| 50187 | 10: 13947449 | 50188 | 10: 13953167 | 50191 | 10: 14062089 | 50201 | 10: 14318815 | 50203 | 10: 14387176 |
| 50204 | 10: 14387540 | 50216 | 10: 14685085 | 50230 | 10: 15045027 | 50231 | 10: 15055444 | 50239 | 10: 15212747 |
| 50241 | 10: 15253352 | 50242 | 10: 15253401 | 50244 | 10: 15306720 | 50250 | 10: 15382216 | 50251 | 10: 15382286 |
| 50254 | 10: 15491085 | 50261 | 10: 15626947 | 50268 | 10: 15765399 | 50269 | 10: 15766293 | 50270 | 10: 15766923 |
| 50271 | 10: 15768048 | 50272 | 10: 15777260 | 50274 | 10: 15872387 | 50275 | 10: 15872397 | 50276 | 10: 15878690 |
| 50277 | 10: 15887011 | 50278 | 10: 15887086 | 50279 | 10: 15888077 | 50280 | 10: 15888157 | 50286 | 10: 15986933 |
| 50287 | 10: 15988001 | 50293 | 10: 16160207 | 50294 | 10: 16160289 | 50295 | 10: 16160311 | 50300 | 10: 16292572 |
| 50303 | 10: 16351611 | 50309 | 10: 16477102 | 50311 | 10: 16505051 | 50315 | 10: 16579579 | 50317 | 10: 16615178 |
| 50318 | 10: 16616715 | 50319 | 10: 16622209 | 50321 | 10: 16657010 | 50323 | 10: 16689144 | 50325 | 10: 16708640 |
| 50329 | 10: 16760750 | 50330 | 10: 16783563 | 50332 | 10: 16840169 | 50333 | 10: 16840192 | 50334 | 10: 16840220 |
| 50346 | 10: 17026997 | 50358 | 10: 17327263 | 50362 | 10: 17375623 | 50364 | 10: 17405609 | 50372 | 10: 17576175 |
| 50380 | 10: 17746328 | 50393 | 10: 18053005 | 50409 | 10: 18204972 | 50413 | 10: 18326097 | 50417 | 10: 18459800 |
| 50429 | 10: 18655176 | 50430 | 10: 18656419 | 50440 | 10: 19055444 | 50441 | 10: 19055478 | 50444 | 10: 19108089 |
| 50445 | 10: 19108125 | 50446 | 10: 19108146 | 50447 | 10: 19108188 | 50448 | 10: 19108578 | 50449 | 10: 19108623 |
| 50450 | 10: 19108629 | 50463 | 10: 19296017 | 50473 | 10: 19582204 | 50483 | 10: 19757013 | 50484 | 10: 19757066 |
| 50485 | 10: 19757109 | 50492 | 10: 19887391 | 50493 | 10: 19887438 | 50494 | 10: 19887463 | 50495 | 10: 19887464 |
| 50496 | 10: 19887506 | 50497 | 10: 19906833 | 50502 | 10: 20032774 | 50522 | 10: 20511602 | 50523 | 10: 20517856 |
| 50531 | 10: 20716836 | 50533 | 10: 20746648 | 50534 | 10: 20749216 | 50535 | 10: 20765182 | 50538 | 10: 20798013 |
| 50539 | 10: 20800521 | 50540 | 10: 20823031 | 50542 | 10: 20851467 | 50543 | 10: 20854079 | 50545 | 10: 20866367 |
| 50546 | 10: 20866872 | 50547 | 10: 20878828 | 50548 | 10: 20887914 | 50549 | 10: 20905201 | 50550 | 10: 20905351 |
| 50551 | 10: 20906045 | 50552 | 10: 20922221 | 50555 | 10: 20962445 | 50556 | 10: 20969179 | 50557 | 10: 20971568 |
| 50558 | 10: 20980278 | 50559 | 10: 20992105 | 50561 | 10: 21041241 | 50564 | 10: 21086459 | 50570 | 10: 21193718 |
| 50581 | 10: 21392700 | 50586 | 10: 21482874 | 50587 | 10: 21494322 | 50588 | 10: 21496846 | 50590 | 10: 21512555 |
| 50602 | 10: 21928460 | 50604 | 10: 21943044 | 50606 | 10: 21954776 | 50607 | 10: 21955205 | 50608 | 10: 21961681 |
| 50609 | 10: 21969630 | 50610 | 10: 21969644 | 50611 | 10: 21969862 | 50612 | 10: 21970634 | 50613 | 10: 21972554 |
| 50620 | 10: 22271591 | 50629 | 10: 22389791 | 50640 | 10: 22796412 | 50644 | 10: 23008980 | 50645 | 10: 23058608 |
| 50685 | 10: 25836315 | 50687 | 10: 25842537 | 50689 | 10: 25851554 | 50707 | 10: 26370686 | 50712 | 10: 26442221 |
| 50716 | 10: 26521248 | 50719 | 10: 26552273 | 50720 | 10: 26552368 | 50725 | 10: 26660705 | 50726 | 10: 26664869 |
| 50728 | 10: 26699741 | 50733 | 10: 26735536 | 50739 | 10: 26806421 | 50744 | 10: 26852657 | 50747 | 10: 26914900 |
| 50749 | 10: 26936311 | 50751 | 10: 26971279 | 50752 | 10: 26979102 | 50753 | 10: 26990817 | 50755 | 10: 27045849 |
| 50756 | 10: 27045893 | 50759 | 10: 27104985 | 50760 | 10: 27110316 | 50761 | 10: 27110398 | 50767 | 10: 27219754 |
| 50768 | 10: 27219761 | 50770 | 10: 27257251 | 50773 | 10: 27354015 | 50774 | 10: 27354260 | 50782 | 10: 27629698 |
| 50786 | 10: 27668710 | 50787 | 10: 27668712 | 50788 | 10: 27668749 | 50805 | 10: 28336043 | 50818 | 10: 28717710 |
| 50822 | 10: 28777408 | 50823 | 10: 28777482 | 50824 | 10: 28777483 | 50825 | 10: 28777517 | 50826 | 10: 28816832 |
| 50828 | 10: 28830145 | 50829 | 10: 28830820 | 50830 | 10: 28831951 | 50831 | 10: 28837592 | 50834 | 10: 28870874 |
| 50837 | 10: 28920327 | 50838 | 10: 28921627 | 50839 | 10: 28922441 | 50841 | 10: 28924088 | 50842 | 10: 28924117 |
| 50846 | 10: 28936523 | 50847 | 10: 28936611 | 50849 | 10: 28939012 | 50851 | 10: 28941456 | 50852 | 10: 28941564 |
| 50853 | 10: 28941645 | 50854 | 10: 28967860 | 50855 | 10: 28967993 | 50865 | 10: 29192994 | 50867 | 10: 29207986 |
| 50875 | 10: 29370574 | 50877 | 10: 29394079 | 50879 | 10: 29415078 | 50880 | 10: 29434816 | 50883 | 10: 29489538 |
| 50887 | 10: 29551880 | 50894 | 10: 29683783 | 50908 | 10: 30087181 | 50918 | 10: 30456072 | 50927 | 10: 30882706 |
| 50929 | 10: 31026079 | 50943 | 10: 31383428 | 50944 | 10: 31395880 | 50946 | 10: 31421937 | 50948 | 10: 31465645 |
| 50950 | 10: 31539600 | 50965 | 10: 31989230 | 50975 | 10: 32311663 | 50987 | 10: 32581894 | 50990 | 10: 32646966 |
| 50993 | 10: 32695364 | 50996 | 10: 32766762 | 51016 | 10: 33233733 | 51023 | 10: 33473275 | 51025 | 10: 33520438 |
| 51038 | 10: 33775393 | 51040 | 10: 33792624 | 51041 | 10: 33803176 | 51052 | 10: 34100470 | 51054 | 10: 34214726 |
| 51056 | 10: 34306045 | 51058 | 10: 34339538 | 51062 | 10: 34390466 | 51063 | 10: 34399556 | 51065 | 10: 34436191 |
| 51066 | 10: 34441644 | 51067 | 10: 34463264 | 51070 | 10: 34502888 | 51078 | 10: 34709456 | 51079 | 10: 34711929 |
| 51084 | 10: 34865113 | 51088 | 10: 34924089 | 51101 | 10: 35135295 | 51104 | 10: 35165655 | 51105 | 10: 35167815 |
| 51107 | 10: 35180569 | 51109 | 10: 35184337 | 51111 | 10: 35210635 | 51112 | 10: 35210945 | 51113 | 10: 35213913 |
| 51114 | 10: 35216558 | 51117 | 10: 35370763 | 51128 | 10: 35779938 | 51129 | 10: 35856231 | 51131 | 10: 35906682 |
| 51132 | 10: 35916585 | 51135 | 10: 35975249 | 51137 | 10: 35982115 | 51143 | 10: 36077032 | 51144 | 10: 36131193 |
| 51151 | 10: 36412382 | 51153 | 10: 36441443 | 51154 | 10: 36441686 | 51166 | 10: 36836677 | 51170 | 10: 36919070 |
| 51172 | 10: 36945666 | 51173 | 10: 36951207 | 51185 | 10: 37239881 | 51188 | 10: 37341433 | 51198 | 10: 37638488 |
| 51212 | 10: 37805187 | 51214 | 10: 37904587 | 51218 | 10: 37944601 | 51229 | 10: 38333207 | 51231 | 10: 38393603 |
| 51236 | 10: 38488614 | 51245 | 10: 38701360 | 51246 | 10: 38753237 | 51267 | 10: 39292295 | 51269 | 10: 39363047 |
| 51271 | 10: 39364510 | 51273 | 10: 39377386 | 51303 | 10: 40330913 | 51306 | 10: 40373055 | 51307 | 10: 40382252 |
| 51308 | 10: 40389854 | 51309 | 10: 40408498 | 51314 | 10: 40556907 | 51321 | 10: 40717249 | 51360 | 10: 41919935 |
| 51369 | 10: 42252004 | 51393 | 10: 42819454 | 51397 | 10: 42948816 | 51406 | 10: 43236868 | 51413 | 10: 43488771 |
| 51425 | 10: 43985352 | 51432 | 10: 44080128 | 51437 | 10: 44252723 | 51446 | 10: 44448699 | 51451 | 10: 44583693 |
| 51456 | 10: 44658796 | 51457 | 10: 44687920 | 51470 | 10: 44966040 | 51472 | 10: 45022368 | 51476 | 10: 45222091 |
| 51482 | 10: 45334197 | 51483 | 10: 45334223 | 51484 | 10: 45334295 | 51485 | 10: 45383083 | 51486 | 10: 45385249 |
| 51487 | 10: 45407393 | 51488 | 10: 45421821 | 51492 | 10: 45548786 | 51502 | 10: 45782999 | 51504 | 10: 45794472 |
| 51505 | 10: 45797020 | 51507 | 10: 45802351 | 51509 | 10: 45807766 | 51510 | 10: 45825756 | 51511 | 10: 45825757 |
| 51512 | 10: 45825760 | 51513 | 10: 45852804 | 51515 | 10: 45888410 | 51517 | 10: 45962441 | 51535 | 10: 46450572 |
| 51552 | 10: 46711744 | 51556 | 10: 46771999 | 51565 | 10: 47008189 | 51572 | 10: 47133365 | 51574 | 10: 47141503 |
| 51576 | 10: 47158682 | 51580 | 10: 47240491 | 51585 | 10: 47306453 | 51589 | 10: 47384963 | 51590 | 10: 47384967 |
| 51598 | 10: 47600619 | 51601 | 10: 47645531 | 51605 | 10: 47674190 | 51608 | 10: 47753050 | 51614 | 10: 48040907 |
| 51632 | 10: 48370815 | 51634 | 10: 48417884 | 51644 | 10: 48589378 | 51646 | 10: 48589442 | 51648 | 10: 48668493 |
| 51650 | 10: 48743669 | 51660 | 10: 49108347 | 51661 | 10: 49132750 | 51668 | 10: 49271034 | 51675 | 10: 49350595 |
| 51677 | 10: 49377221 | 51678 | 10: 49377286 | 51681 | 10: 49419982 | 51693 | 10: 49601513 | 51703 | 10: 49841256 |
| 51704 | 10: 49841837 | 51705 | 10: 49858694 | 51715 | 10: 50107685 | 51734 | 10: 50489426 | 51739 | 10: 50557454 |
| 51740 | 10: 50582984 | 51746 | 10: 50846288 | 51749 | 10: 51020894 | 51752 | 10: 51062674 | 51757 | 10: 51161988 |
| 51758 | 10: 51163453 | 51761 | 10: 51186753 | 51771 | 10: 51429834 | 51772 | 10: 51436172 | 51776 | 10: 51594174 |
| 51786 | 10: 51771324 | 51787 | 10: 51772122 | 51798 | 10: 52037658 | 51799 | 10: 52053671 | 51807 | 10: 52239885 |
| 51808 | 10: 52247304 | 51818 | 10: 52549691 | 51820 | 10: 52577136 | 51821 | 10: 52644574 | 51826 | 10: 52759280 |
| 51827 | 10: 52770881 | 51841 | 10: 52889989 | 51843 | 10: 52975439 | 51852 | 10: 53392084 | 51855 | 10: 53503163 |
| 51856 | 10: 53566836 | 51857 | 10: 53573889 | 51859 | 10: 53599523 | 51870 | 10: 54084338 | 51871 | 10: 54097737 |
| 51873 | 10: 54151043 | 51874 | 10: 54166847 | 51879 | 10: 54206436 | 51880 | 10: 54223827 | 51885 | 10: 54381985 |
| 51897 | 10: 54587863 | 51900 | 10: 54728840 | 51905 | 10: 55049581 | 51908 | 10: 55137727 | 51909 | 10: 55138450 |
| 51915 | 10: 55257378 | 51922 | 10: 55519496 | 51928 | 10: 55693450 | 51929 | 10: 55701600 | 51930 | 10: 55701734 |
| 51935 | 10: 55824628 | 51937 | 10: 55884610 | 51944 | 10: 56050425 | 51950 | 10: 56235551 | 51961 | 10: 56404358 |
| 51974 | 10: 56691062 | 52008 | 10: 57738832 | 52009 | 10: 57749028 | 52012 | 10: 57825646 | 52021 | 10: 58035128 |
| 52030 | 10: 58215720 | 52031 | 10: 58215742 | 52032 | 10: 58223900 | 52033 | 10: 58223933 | 52034 | 10: 58223992 |
| 52038 | 10: 58382992 | 52043 | 10: 58575796 | 52048 | 10: 58651910 | 52055 | 10: 58892988 | 52058 | 10: 58957454 |
| 52059 | 10: 58957455 | 52060 | 10: 58957462 | 52061 | 10: 58963341 | 52068 | 10: 59169515 | 52069 | 10: 59257696 |
| 52070 | 10: 59257779 | 52074 | 10: 59431442 | 52084 | 10: 59743414 | 52096 | 10: 60229145 | 52099 | 10: 60376158 |
| 52104 | 10: 60467179 | 52132 | 10: 61502794 | 52137 | 10: 61691415 | 52142 | 10: 61759883 | 52143 | 10: 61761276 |
| 52144 | 10: 61770176 | 52147 | 10: 61801949 | 52152 | 10: 61854226 | 52156 | 10: 61927985 | 52161 | 10: 62065140 |
| 52162 | 10: 62070975 | 52167 | 10: 62152633 | 52188 | 10: 62478919 | 52189 | 10: 62483660 | 52196 | 10: 62841361 |
| 52197 | 10: 62843919 | 52206 | 10: 63003189 | 52207 | 10: 63005018 | 52215 | 10: 63176255 | 52217 | 10: 63234235 |
| 52218 | 10: 63236141 | 52230 | 10: 63545031 | 52233 | 10: 63676002 | 52235 | 10: 63714642 | 52246 | 10: 64098165 |
| 52254 | 10: 64366724 | 52255 | 10: 64366769 | 52256 | 10: 64366784 | 52257 | 10: 64366788 | 52258 | 10: 64366808 |
| 52259 | 10: 64366813 | 52260 | 10: 64366843 | 52273 | 10: 64623835 | 52274 | 10: 64624712 | 52297 | 10: 65311772 |
| 52298 | 10: 65333523 | 52300 | 10: 65360982 | 52301 | 10: 65363558 | 52302 | 10: 65366448 | 52303 | 10: 65370682 |
| 52304 | 10: 65374137 | 52305 | 10: 65381281 | 52306 | 10: 65388071 | 52307 | 10: 65427744 | 52308 | 10: 65428255 |
| 52309 | 10: 65448723 | 52310 | 10: 65460822 | 52311 | 10: 65464926 | 52315 | 10: 65667285 | 52317 | 10: 65689392 |
| 52319 | 10: 65693892 | 52320 | 10: 65705467 | 52328 | 10: 65975774 | 52333 | 10: 66037974 | 52374 | 10: 66920995 |
| 52379 | 10: 67082677 | 52383 | 10: 67193245 | 52384 | 10: 67193602 | 52385 | 10: 67207585 | 52388 | 10: 67271539 |
| 52389 | 10: 67275755 | 52400 | 10: 67706373 | 52401 | 10: 67706382 | 52403 | 10: 67837002 | 52412 | 10: 67919519 |
| 52415 | 10: 68009703 | 52419 | 10: 68069027 | 52426 | 10: 68164248 | 52430 | 10: 68213913 | 52434 | 10: 68307915 |
| 52435 | 10: 68336932 | 52445 | 10: 68532314 | 52446 | 10: 68532338 | 52449 | 10: 68589455 | 52453 | 10: 68616316 |
| 52454 | 10: 68648835 | 52460 | 10: 68845801 | 52479 | 10: 69314335 | 52483 | 10: 69414869 | 52489 | 10: 69550619 |
| 52497 | 10: 69813170 | 52506 | 10: 69985799 | 52518 | 10: 70311136 | 52530 | 10: 70914151 | 52532 | 10: 70933695 |
| 52543 | 10: 71144557 | 52545 | 10: 71190058 | 52547 | 10: 71282860 | 52549 | 10: 71395114 | 52550 | 10: 71401946 |
| 52553 | 10: 71492246 | 52560 | 10: 71739567 | 52561 | 10: 71739616 | 52562 | 10: 71741589 | 52568 | 10: 71990029 |
| 52594 | 10: 72844951 | 52601 | 10: 73180235 | 52603 | 10: 73207644 | 52608 | 10: 73381610 | 52619 | 10: 73593430 |
| 52621 | 10: 73621273 | 52625 | 10: 73665667 | 52628 | 10: 73702186 | 52631 | 10: 73800772 | 52634 | 10: 73889706 |
| 52639 | 10: 74014694 | 52640 | 10: 74014740 | 52647 | 10: 74278788 | 52669 | 10: 75144155 | 52684 | 10: 75472993 |
| 52690 | 10: 75648302 | 52702 | 10: 76063641 | 52711 | 10: 76208279 | 52712 | 10: 76213571 | 52713 | 10: 76216092 |
| 52714 | 10: 76217503 | 52716 | 10: 76219150 | 52717 | 10: 76231514 | 52727 | 10: 76445843 | 52739 | 10: 76742309 |
| 52740 | 10: 76780386 | 52750 | 10: 76927979 | 52753 | 10: 77027256 | 52754 | 10: 77038645 | 52755 | 10: 77043267 |
| 52756 | 10: 77050549 | 52757 | 10: 77063990 | 52758 | 10: 77079521 | 52760 | 10: 77086192 | 52761 | 10: 77087944 |
| 52764 | 10: 77165808 | 52765 | 10: 77182957 | 52769 | 10: 77284463 | 52770 | 10: 77295759 | 52772 | 10: 77310958 |
| 52783 | 10: 77734390 | 52785 | 10: 77815166 | 52787 | 10: 77843400 | 52790 | 10: 77908553 | 52794 | 10: 77934474 |
| 52800 | 10: 78024784 | 52804 | 10: 78090692 | 52805 | 10: 78091383 | 52806 | 10: 78170657 | 52818 | 10: 78466873 |
| 52819 | 10: 78489026 | 52820 | 10: 78524325 | 52833 | 10: 78874750 | 52834 | 10: 78890533 | 52843 | 10: 79142128 |
| 52844 | 10: 79142222 | 52850 | 10: 79364696 | 52858 | 10: 79552918 | 52864 | 10: 79765450 | 52866 | 10: 79778751 |
| 52876 | 10: 80036481 | 52878 | 10: 80058483 | 52887 | 10: 80321841 | 52888 | 10: 80354807 | 52890 | 10: 80376119 |
| 52897 | 10: 80516438 | 52898 | 10: 80584353 | 52899 | 10: 80612799 | 52908 | 10: 80833008 | 52917 | 10: 81113106 |
| 52918 | 10: 81113113 | 52919 | 10: 81113154 | 52920 | 10: 81119799 | 52921 | 10: 81122203 | 52922 | 10: 81122225 |
| 52923 | 10: 81133087 | 52924 | 10: 81134221 | 52925 | 10: 81141810 | 52926 | 10: 81147906 | 52927 | 10: 81159861 |
| 52928 | 10: 81163746 | 52929 | 10: 81169707 | 52930 | 10: 81171404 | 52931 | 10: 81174589 | 52932 | 10: 81177743 |
| 52933 | 10: 81179444 | 52934 | 10: 81180179 | 52935 | 10: 81182426 | 52936 | 10: 81188153 | 52937 | 10: 81191624 |
| 52938 | 10: 81192985 | 52939 | 10: 81201260 | 52942 | 10: 81232104 | 52946 | 10: 81275274 | 52948 | 10: 81293569 |
| 52949 | 10: 81313921 | 52954 | 10: 81424405 | 52955 | 10: 81429276 | 52956 | 10: 81433859 | 52957 | 10: 81434033 |
| 52960 | 10: 81522739 | 52961 | 10: 81563853 | 52971 | 10: 81760421 | 52983 | 10: 81884426 | 52985 | 10: 81907059 |
| 52989 | 10: 82020430 | 52991 | 10: 82046798 | 52993 | 10: 82113076 | 52994 | 10: 82117432 | 52996 | 10: 82155665 |
| 53001 | 10: 82226992 | 53003 | 10: 82262769 | 53010 | 10: 82436819 | 53020 | 10: 82813082 | 53031 | 10: 83035771 |
| 53036 | 10: 83168495 | 53037 | 10: 83180678 | 53043 | 10: 83268162 | 53064 | 10: 83783323 | 53074 | 10: 83967539 |
| 53078 | 10: 84046423 | 53080 | 10: 84074336 | 53081 | 10: 84113189 | 53087 | 10: 84235120 | 53088 | 10: 84249302 |
| 53091 | 10: 84274673 | 53093 | 10: 84321770 | 53095 | 10: 84358684 | 53098 | 10: 84410074 | 53101 | 10: 84432430 |
| 53113 | 10: 84804439 | 53114 | 10: 84809599 | 53117 | 10: 84921371 | 53120 | 10: 84937878 | 53122 | 10: 84972916 |
| 53124 | 10: 85090540 | 53125 | 10: 85090775 | 53126 | 10: 85093417 | 53129 | 10: 85140109 | 53135 | 10: 85210872 |
| 53139 | 10: 85277645 | 53144 | 10: 85348496 | 53148 | 10: 85452360 | 53159 | 10: 85646771 | 53161 | 10: 85685359 |
| 53162 | 10: 85714779 | 53163 | 10: 85731082 | 53175 | 10: 86198306 | 53179 | 10: 86295239 | 53180 | 10: 86322591 |
| 53182 | 10: 86350641 | 53183 | 10: 86373524 | 53186 | 10: 86404676 | 53188 | 10: 86427269 | 53191 | 10: 86481134 |
| 53193 | 10: 86492591 | 53194 | 10: 86510757 | 53196 | 10: 86545729 | 53197 | 10: 86553401 | 53207 | 10: 86743362 |
| 53209 | 10: 86835796 | 53211 | 10: 86863386 | 53214 | 10: 86939644 | 53223 | 10: 87113458 | 53244 | 10: 87621927 |
| 53246 | 10: 87627438 | 53250 | 10: 87721184 | 53252 | 10: 87755864 | 53256 | 10: 87849396 | 53257 | 10: 87849463 |
| 53258 | 10: 87850909 | 53268 | 10: 88187681 | 53270 | 10: 88260182 | 53271 | 10: 88298292 | 53274 | 10: 88347395 |
| 53276 | 10: 88380317 | 53280 | 10: 88522805 | 53291 | 10: 88839037 | 53292 | 10: 88845164 | 53293 | 10: 88846191 |
| 53297 | 10: 88888537 | 53299 | 10: 88925587 | 53313 | 10: 89160492 | 53314 | 10: 89160521 | 53316 | 10: 89233565 |
| 53331 | 10: 89626724 | 53332 | 10: 89626819 | 53343 | 10: 89798617 | 53348 | 10: 89872058 | 53349 | 10: 89872136 |
| 53350 | 10: 89933077 | 53364 | 10: 90186331 | 53378 | 10: 90418700 | 53379 | 10: 90418707 | 53415 | 10: 90987301 |
| 53419 | 10: 91011354 | 53420 | 10: 91019729 | 53431 | 10: 91348121 | 53444 | 10: 91737619 | 53445 | 10: 91746178 |
| 53449 | 10: 91823694 | 53456 | 10: 92080780 | 53458 | 10: 92100804 | 53464 | 10: 92232897 | 53465 | 10: 92281587 |
| 53473 | 10: 92466754 | 53476 | 10: 92499113 | 53480 | 10: 92594602 | 53482 | 10: 92604984 | 53488 | 10: 92710477 |
| 53489 | 10: 92717162 | 53492 | 10: 92799252 | 53507 | 10: 93053048 | 53508 | 10: 93053064 | 53512 | 10: 93140659 |
| 53528 | 10: 93626548 | 53537 | 10: 93975527 | 53538 | 10: 93977121 | 53549 | 10: 94274383 | 53557 | 10: 94377341 |
| 53558 | 10: 94377386 | 53572 | 10: 94695544 | 53576 | 10: 94786454 | 53584 | 10: 95064914 | 53595 | 10: 95385183 |
| 53597 | 10: 95412469 | 53602 | 10: 95561991 | 53603 | 10: 95575347 | 53608 | 10: 95643374 | 53609 | 10: 95672545 |
| 53611 | 10: 95688501 | 53613 | 10: 95728606 | 53618 | 10: 95843138 | 53620 | 10: 95843176 | 53628 | 10: 96029745 |
| 53635 | 10: 96240911 | 53638 | 10: 96288699 | 53641 | 10: 96380513 | 53642 | 10: 96380599 | 53647 | 10: 96569247 |
| 53650 | 10: 96598589 | 53657 | 10: 96839484 | 53659 | 10: 96875821 | 53662 | 10: 96976508 | 53673 | 10: 97153819 |
| 53678 | 10: 97206516 | 53683 | 10: 97309948 | 53684 | 10: 97309954 | 53692 | 10: 97454671 | 53699 | 10: 97550303 |
| 53700 | 10: 97582963 | 53749 | 10: 98562531 | 53777 | 10: 99221264 | 53788 | 10: 99540676 | 53789 | 10: 99540722 |
| 53790 | 10: 99540732 | 53791 | 10: 99540816 | 53792 | 10: 99540849 | 53805 | 10: 99922832 | 53806 | 10: 99926816 |
| 53808 | 10: 99941485 | 53812 | 10: 10001067 | 53815 | 10: 10008239 | 53819 | 10: 10018045 | 53820 | 10: 10018777 |
| 53822 | 10: 10024360 | 53825 | 10: 10029136 | 53826 | 10: 10030147 | 53827 | 10: 10030338 | 53828 | 10: 10030391 |
| 53829 | 10: 10030447 | 53831 | 10: 10033080 | 53852 | 10: 10082168 | 53854 | 10: 10087653 | 53858 | 10: 10096073 |
| 53860 | 10: 10096399 | 53861 | 10: 10097431 | 53871 | 10: 10113021 | 53872 | 10: 10113562 | 53888 | 10: 10143538 |
| 53889 | 10: 10147495 | 53890 | 10: 10148861 | 53891 | 10: 10150021 | 53892 | 10: 10150656 | 53903 | 10: 10170837 |
| 53912 | 10: 10180575 | 53918 | 10: 10186280 | 53919 | 10: 10187041 | 53924 | 10: 10200983 | 53934 | 10: 10213583 |
| 53937 | 10: 10221227 | 53938 | 10: 10222090 | 53940 | 10: 10225283 | 53947 | 10: 10236108 | 53950 | 10: 10248171 |
| 53959 | 10: 10271339 | 53962 | 10: 10281920 | 54004 | 11: 441458 | 54008 | 11: 588104 | 54009 | 11: 612918 |
| 54010 | 11: 620469 | 54013 | 11: 675675 | 54015 | 11: 721258 | 54018 | 11: 743177 | 54023 | 11: 818693 |
| 54024 | 11: 818745 | 54025 | 11: 839682 | 54028 | 11: 884789 | 54029 | 11: 916613 | 54042 | 11: 1140277 |
| 54051 | 11: 1252661 | 54072 | 11: 1599964 | 54073 | 11: 1603522 | 54074 | 11: 1604088 | 54076 | 11: 1610503 |
| 54077 | 11: 1613029 | 54078 | 11: 1613069 | 54079 | 11: 1615691 | 54080 | 11: 1615825 | 54081 | 11: 1615860 |
| 54082 | 11: 1616045 | 54083 | 11: 1617470 | 54084 | 11: 1617491 | 54085 | 11: 1618013 | 54088 | 11: 1661775 |
| 54090 | 11: 1692551 | 54091 | 11: 1692581 | 54092 | 11: 1692586 | 54094 | 11: 1715577 | 54105 | 11: 1929102 |
| 54106 | 11: 1939516 | 54109 | 11: 1975548 | 54112 | 11: 2019047 | 54113 | 11: 2019051 | 54116 | 11: 2054152 |
| 54117 | 11: 2058128 | 54122 | 11: 2135119 | 54123 | 11: 2165271 | 54124 | 11: 2165552 | 54125 | 11: 2165653 |
| 54135 | 11: 2371904 | 54136 | 11: 2375767 | 54137 | 11: 2379791 | 54138 | 11: 2380701 | 54139 | 11: 2381359 |
| 54141 | 11: 2381820 | 54142 | 11: 2381938 | 54143 | 11: 2382261 | 54144 | 11: 2383322 | 54145 | 11: 2383432 |
| 54147 | 11: 2388794 | 54148 | 11: 2391923 | 54149 | 11: 2392633 | 54150 | 11: 2392687 | 54151 | 11: 2392744 |
| 54152 | 11: 2393551 | 54153 | 11: 2393601 | 54154 | 11: 2393614 | 54155 | 11: 2394563 | 54156 | 11: 2394565 |
| 54157 | 11: 2394754 | 54158 | 11: 2429820 | 54159 | 11: 2433742 | 54162 | 11: 2466683 | 54163 | 11: 2466765 |
| 54164 | 11: 2466767 | 54176 | 11: 2690324 | 54177 | 11: 2691733 | 54179 | 11: 2704089 | 54185 | 11: 2887185 |
| 54186 | 11: 2909443 | 54190 | 11: 2965363 | 54191 | 11: 2970198 | 54192 | 11: 2971406 | 54193 | 11: 2974009 |
| 54194 | 11: 3048024 | 54195 | 11: 3048066 | 54197 | 11: 3068135 | 54198 | 11: 3073771 | 54201 | 11: 3080087 |
| 54202 | 11: 3080140 | 54203 | 11: 3080321 | 54204 | 11: 3080329 | 54212 | 11: 3324474 | 54214 | 11: 3339436 |
| 54222 | 11: 3496384 | 54223 | 11: 3498026 | 54229 | 11: 3630171 | 54232 | 11: 3683284 | 54234 | 11: 3687500 |
| 54235 | 11: 3697811 | 54236 | 11: 3703164 | 54241 | 11: 3827658 | 54242 | 11: 3831757 | 54243 | 11: 3831764 |
| 54244 | 11: 3833403 | 54245 | 11: 3834470 | 54246 | 11: 3834544 | 54249 | 11: 3860402 | 54250 | 11: 3861478 |
| 54251 | 11: 3861495 | 54252 | 11: 3863319 | 54258 | 11: 4044668 | 54261 | 11: 4144540 | 54274 | 11: 4626193 |
| 54275 | 11: 4643628 | 54277 | 11: 4692004 | 54278 | 11: 4715218 | 54280 | 11: 4754025 | 54285 | 11: 4846290 |
| 54288 | 11: 4908167 | 54301 | 11: 5122618 | 54306 | 11: 5207458 | 54307 | 11: 5208484 | 54308 | 11: 5216864 |
| 54317 | 11: 5475369 | 54320 | 11: 5522837 | 54321 | 11: 5546069 | 54322 | 11: 5547645 | 54336 | 11: 5892298 |
| 54338 | 11: 5921461 | 54342 | 11: 6014407 | 54344 | 11: 6057385 | 54346 | 11: 6090351 | 54353 | 11: 6281176 |
| 54367 | 11: 6672389 | 54371 | 11: 6825527 | 54378 | 11: 7079505 | 54379 | 11: 7079550 | 54383 | 11: 7226247 |
| 54385 | 11: 7290568 | 54387 | 11: 7312202 | 54390 | 11: 7398056 | 54392 | 11: 7442481 | 54395 | 11: 7492967 |
| 54397 | 11: 7506651 | 54406 | 11: 7612707 | 54424 | 11: 8016040 | 54441 | 11: 8559109 | 54456 | 11: 9088843 |
| 54457 | 11: 9094665 | 54458 | 11: 9108482 | 54459 | 11: 9116906 | 54460 | 11: 9122303 | 54461 | 11: 9124299 |
| 54462 | 11: 9125436 | 54463 | 11: 9129918 | 54464 | 11: 9140181 | 54465 | 11: 9142056 | 54467 | 11: 9146703 |
| 54468 | 11: 9147988 | 54469 | 11: 9149728 | 54470 | 11: 9150188 | 54471 | 11: 9150797 | 54472 | 11: 9152234 |
| 54473 | 11: 9153632 | 54477 | 11: 9263561 | 54485 | 11: 9432577 | 54493 | 11: 9646962 | 54497 | 11: 9717817 |
| 54499 | 11: 9759593 | 54502 | 11: 9782467 | 54509 | 11: 9926241 | 54516 | 11: 10139900 | 54517 | 11: 10192082 |
| 54518 | 11: 10205888 | 54519 | 11: 10207241 | 54521 | 11: 10218640 | 54522 | 11: 10223170 | 54528 | 11: 10378672 |
| 54534 | 11: 10498811 | 54535 | 11: 10506565 | 54539 | 11: 10583951 | 54542 | 11: 10706952 | 54543 | 11: 10730447 |
| 54545 | 11: 10770592 | 54547 | 11: 10787415 | 54548 | 11: 10804935 | 54549 | 11: 10814646 | 54551 | 11: 10817795 |
| 54559 | 11: 11049635 | 54562 | 11: 11215341 | 54564 | 11: 11231984 | 54565 | 11: 11242289 | 54573 | 11: 11352441 |
| 54574 | 11: 11362173 | 54576 | 11: 11392077 | 54578 | 11: 11435064 | 54584 | 11: 11556298 | 54586 | 11: 11616272 |
| 54601 | 11: 12092660 | 54602 | 11: 12096347 | 54608 | 11: 12209379 | 54623 | 11: 12569024 | 54625 | 11: 12600200 |
| 54634 | 11: 12790767 | 54643 | 11: 13030163 | 54645 | 11: 13087614 | 54648 | 11: 13127507 | 54662 | 11: 13410435 |
| 54667 | 11: 13471629 | 54693 | 11: 14069498 | 54697 | 11: 14240512 | 54700 | 11: 14312963 | 54707 | 11: 14569196 |
| 54708 | 11: 14601805 | 54717 | 11: 14951793 | 54720 | 11: 15049901 | 54729 | 11: 15188618 | 54737 | 11: 15351949 |
| 54800 | 11: 16839124 | 54806 | 11: 17005906 | 54808 | 11: 17038164 | 54809 | 11: 17038230 | 54811 | 11: 17062733 |
| 54819 | 11: 17340238 | 54820 | 11: 17340240 | 54821 | 11: 17361542 | 54823 | 11: 17465587 | 54829 | 11: 17736027 |
| 54830 | 11: 17736142 | 54870 | 11: 18811490 | 54871 | 11: 18834617 | 54879 | 11: 19016954 | 54885 | 11: 19227167 |
| 54891 | 11: 19479022 | 54893 | 11: 19516116 | 54894 | 11: 19516281 | 54897 | 11: 19574521 | 54909 | 11: 20009995 |
| 54914 | 11: 20249271 | 54916 | 11: 20268750 | 54929 | 11: 20671302 | 54933 | 11: 20761364 | 54935 | 11: 20839310 |
| 54946 | 11: 21100669 | 54948 | 11: 21153847 | 54952 | 11: 21298331 | 54953 | 11: 21386222 | 54965 | 11: 21549331 |
| 54968 | 11: 21639708 | 54971 | 11: 21679170 | 54979 | 11: 21792131 | 54985 | 11: 21911853 | 54998 | 11: 22232061 |
| 54999 | 11: 22277590 | 55009 | 11: 22536318 | 55016 | 11: 22632697 | 55031 | 11: 23047670 | 55035 | 11: 23109116 |
| 55043 | 11: 23406318 | 55045 | 11: 23420733 | 55057 | 11: 23678147 | 55059 | 11: 23713701 | 55060 | 11: 23740579 |
| 55061 | 11: 23792727 | 55062 | 11: 23799914 | 55072 | 11: 23927783 | 55073 | 11: 23927808 | 55082 | 11: 24309077 |
| 55114 | 11: 24847692 | 55115 | 11: 24847723 | 55120 | 11: 25005279 | 55143 | 11: 25650622 | 55144 | 11: 25668988 |
| 55155 | 11: 26000399 | 55158 | 11: 26101828 | 55163 | 11: 26155816 | 55171 | 11: 26293504 | 55172 | 11: 26294033 |
| 55175 | 11: 26405127 | 55176 | 11: 26406745 | 55179 | 11: 26441367 | 55184 | 11: 26597807 | 55202 | 11: 27150645 |
| 55210 | 11: 27309958 | 55223 | 11: 27583040 | 55224 | 11: 27612153 | 55225 | 11: 27648326 | 55226 | 11: 27664648 |
| 55227 | 11: 27673338 | 55235 | 11: 27863723 | 55243 | 11: 28050164 | 55244 | 11: 28054041 | 55254 | 11: 28269292 |
| 55255 | 11: 28312159 | 55256 | 11: 28314792 | 55259 | 11: 28347672 | 55261 | 11: 28406003 | 55270 | 11: 28648699 |
| 55271 | 11: 28677976 | 55272 | 11: 28697612 | 55275 | 11: 28750175 | 55288 | 11: 28998567 | 55292 | 11: 29029776 |
| 55294 | 11: 29094181 | 55305 | 11: 29333853 | 55306 | 11: 29339273 | 55311 | 11: 29433875 | 55312 | 11: 29446589 |
| 55314 | 11: 29483268 | 55316 | 11: 29523821 | 55320 | 11: 29644262 | 55321 | 11: 29652000 | 55328 | 11: 29750755 |
| 55338 | 11: 29944937 | 55339 | 11: 30022269 | 55347 | 11: 30283476 | 55357 | 11: 30480608 | 55358 | 11: 30487514 |
| 55359 | 11: 30490953 | 55360 | 11: 30498423 | 55363 | 11: 30695691 | 55371 | 11: 30816430 | 55375 | 11: 30852665 |
| 55378 | 11: 30909842 | 55381 | 11: 31003265 | 55386 | 11: 31096059 | 55394 | 11: 31338792 | 55395 | 11: 31338818 |
| 55396 | 11: 31338832 | 55418 | 11: 31989218 | 55419 | 11: 32020262 | 55427 | 11: 32309370 | 55440 | 11: 32659947 |
| 55451 | 11: 33038916 | 55454 | 11: 33061320 | 55455 | 11: 33063315 | 55465 | 11: 33230608 | 55473 | 11: 33483917 |
| 55497 | 11: 34161881 | 55512 | 11: 34992407 | 55515 | 11: 35028247 | 55518 | 11: 35081009 | 55523 | 11: 35271551 |
| 55550 | 11: 35973444 | 55555 | 11: 36103640 | 55567 | 11: 36415088 | 55575 | 11: 36677962 | 55578 | 11: 36729555 |
| 55579 | 11: 36730226 | 55584 | 11: 36935209 | 55590 | 11: 37042393 | 55592 | 11: 37065356 | 55593 | 11: 37084548 |
| 55597 | 11: 37151685 | 55602 | 11: 37273838 | 55607 | 11: 37321094 | 55610 | 11: 37357247 | 55617 | 11: 37555217 |
| 55622 | 11: 37622807 | 55623 | 11: 37622964 | 55629 | 11: 37686699 | 55631 | 11: 37699061 | 55632 | 11: 37708915 |
| 55639 | 11: 37993013 | 55640 | 11: 38023147 | 55644 | 11: 38167213 | 55652 | 11: 38524637 | 55676 | 11: 39146746 |
| 55698 | 11: 39953399 | 55719 | 11: 40780484 | 55720 | 11: 40781022 | 55725 | 11: 40977806 | 55729 | 11: 41081305 |
| 55731 | 11: 41095065 | 55732 | 11: 41108333 | 55733 | 11: 41112618 | 55734 | 11: 41119005 | 55735 | 11: 41119970 |
| 55737 | 11: 41122253 | 55738 | 11: 41130588 | 55739 | 11: 41131422 | 55740 | 11: 41138457 | 55742 | 11: 41163401 |
| 55743 | 11: 41165333 | 55744 | 11: 41172319 | 55745 | 11: 41190502 | 55746 | 11: 41211212 | 55747 | 11: 41236765 |
| 55757 | 11: 41353565 | 55785 | 11: 42015177 | 55813 | 11: 42568364 | 55819 | 11: 42700906 | 55838 | 11: 43239984 |
| 55862 | 11: 43925979 | 55864 | 11: 44029565 | 55869 | 11: 44191991 | 55875 | 11: 44335233 | 55877 | 11: 44398012 |
| 55884 | 11: 44616476 | 55885 | 11: 44669598 | 55886 | 11: 44720675 | 55891 | 11: 44864177 | 55892 | 11: 44872149 |
| 55895 | 11: 44907130 | 55896 | 11: 44910831 | 55897 | 11: 44915340 | 55899 | 11: 44947747 | 55900 | 11: 44960989 |
| 55901 | 11: 44979580 | 55905 | 11: 45085243 | 55910 | 11: 45233285 | 55912 | 11: 45240869 | 55926 | 11: 45875224 |
| 55930 | 11: 45909750 | 55932 | 11: 45965043 | 55935 | 11: 46005981 | 55939 | 11: 46042064 | 55944 | 11: 46118783 |
| 55948 | 11: 46189212 | 55966 | 11: 46586822 | 55984 | 11: 46788369 | 55985 | 11: 46796356 | 55986 | 11: 46801200 |
| 55990 | 11: 46887994 | 55991 | 11: 46893010 | 55992 | 11: 46895379 | 55993 | 11: 46895384 | 55994 | 11: 46895386 |
| 55995 | 11: 46895443 | 55996 | 11: 46896988 | 55997 | 11: 46914387 | 55998 | 11: 46915926 | 56001 | 11: 46933431 |
| 56002 | 11: 46946293 | 56009 | 11: 47133868 | 56010 | 11: 47136307 | 56012 | 11: 47153161 | 56016 | 11: 47318477 |
| 56020 | 11: 47346660 | 56023 | 11: 47402450 | 56025 | 11: 47421701 | 56028 | 11: 47484468 | 56039 | 11: 47868107 |
| 56041 | 11: 47957513 | 56051 | 11: 48153310 | 56052 | 11: 48179620 | 56055 | 11: 48232533 | 56057 | 11: 48284436 |
| 56064 | 11: 48495828 | 56065 | 11: 48519034 | 56066 | 11: 48519745 | 56077 | 11: 48624594 | 56078 | 11: 48641618 |
| 56079 | 11: 48643769 | 56080 | 11: 48648683 | 56081 | 11: 48652079 | 56082 | 11: 48653016 | 56083 | 11: 48653703 |
| 56084 | 11: 48659221 | 56086 | 11: 48661468 | 56087 | 11: 48662095 | 56088 | 11: 48663888 | 56089 | 11: 48664547 |
| 56090 | 11: 48665440 | 56091 | 11: 48666103 | 56092 | 11: 48666658 | 56093 | 11: 48673397 | 56094 | 11: 48673919 |
| 56095 | 11: 48674533 | 56096 | 11: 48675652 | 56097 | 11: 48676148 | 56098 | 11: 48677162 | 56099 | 11: 48679806 |
| 56100 | 11: 48680593 | 56101 | 11: 48682804 | 56102 | 11: 48685811 | 56103 | 11: 48690618 | 56104 | 11: 48695696 |
| 56105 | 11: 48703362 | 56107 | 11: 48712410 | 56108 | 11: 48715777 | 56109 | 11: 48729623 | 56110 | 11: 48732364 |
| 56111 | 11: 48740101 | 56112 | 11: 48740643 | 56113 | 11: 48742231 | 56114 | 11: 48745355 | 56115 | 11: 48748086 |
| 56116 | 11: 48757634 | 56117 | 11: 48788737 | 56118 | 11: 48792299 | 56120 | 11: 48910719 | 56134 | 11: 49167307 |
| 56142 | 11: 49394511 | 56144 | 11: 49406127 | 56146 | 11: 49419581 | 56151 | 11: 49574160 | 56163 | 11: 49832758 |
| 56164 | 11: 49852553 | 56165 | 11: 49852648 | 56200 | 11: 51131191 | 56203 | 11: 51184125 | 56220 | 11: 51745809 |
| 56243 | 11: 52697360 | 56265 | 11: 53468115 | 56274 | 11: 53833063 | 56290 | 11: 54327571 | 56298 | 11: 54566473 |
| 56300 | 11: 54590112 | 56320 | 11: 54776336 | 56323 | 11: 54807232 | 56328 | 11: 54930465 | 56329 | 11: 54961590 |
| 56340 | 11: 55186748 | 56347 | 11: 55369267 | 56355 | 11: 55552815 | 56356 | 11: 55571320 | 56363 | 11: 55683959 |
| 56364 | 11: 55696252 | 56378 | 11: 56190217 | 56402 | 11: 56970533 | 56428 | 11: 57877493 | 56439 | 11: 58140156 |
| 56493 | 11: 59622606 | 56497 | 11: 59747111 | 56500 | 11: 59861239 | 56510 | 11: 60271069 | 56511 | 11: 60271074 |
| 56512 | 11: 60271117 | 56513 | 11: 60271193 | 56520 | 11: 60465745 | 56521 | 11: 60465915 | 56522 | 11: 60465920 |
| 56523 | 11: 60465923 | 56524 | 11: 60468952 | 56525 | 11: 60470373 | 56526 | 11: 60470388 | 56527 | 11: 60471668 |
| 56529 | 11: 60472081 | 56530 | 11: 60474103 | 56531 | 11: 60474134 | 56532 | 11: 60474983 | 56533 | 11: 60475508 |
| 56534 | 11: 60475664 | 56535 | 11: 60478021 | 56536 | 11: 60478028 | 56537 | 11: 60478049 | 56538 | 11: 60478204 |
| 56540 | 11: 60494769 | 56541 | 11: 60494791 | 56542 | 11: 60517676 | 56543 | 11: 60566671 | 56544 | 11: 60567347 |
| 56545 | 11: 60567387 | 56546 | 11: 60616886 | 56549 | 11: 60641170 | 56550 | 11: 60641182 | 56553 | 11: 60671076 |
| 56557 | 11: 60758092 | 56558 | 11: 60775322 | 56560 | 11: 60906972 | 56562 | 11: 60949119 | 56564 | 11: 60961745 |
| 56566 | 11: 61011277 | 56567 | 11: 61011512 | 56570 | 11: 61079196 | 56573 | 11: 61139067 | 56574 | 11: 61149300 |
| 56575 | 11: 61149340 | 56576 | 11: 61149341 | 56578 | 11: 61149398 | 56579 | 11: 61149409 | 56581 | 11: 61172809 |
| 56585 | 11: 61361997 | 56588 | 11: 61539166 | 56590 | 11: 61548155 | 56606 | 11: 62153933 | 56616 | 11: 62410952 |
| 56621 | 11: 62549111 | 56627 | 11: 62639185 | 56629 | 11: 62703391 | 56637 | 11: 62848946 | 56645 | 11: 63079035 |
| 56647 | 11: 63132879 | 56657 | 11: 63363010 | 56658 | 11: 63376645 | 56661 | 11: 63394494 | 56665 | 11: 63456586 |
| 56671 | 11: 63645957 | 56672 | 11: 63664952 | 56676 | 11: 63733057 | 56679 | 11: 63799850 | 56687 | 11: 63862724 |
| 56691 | 11: 63954830 | 56693 | 11: 63980633 | 56694 | 11: 63980697 | 56695 | 11: 64007710 | 56708 | 11: 64345353 |
| 56712 | 11: 64472503 | 56716 | 11: 64576376 | 56718 | 11: 64684536 | 56721 | 11: 64807816 | 56726 | 11: 64972478 |
| 56735 | 11: 65170349 | 56736 | 11: 65170393 | 56741 | 11: 65341010 | 56743 | 11: 65372142 | 56746 | 11: 65438305 |
| 56766 | 11: 65998851 | 56769 | 11: 66070592 | 56775 | 11: 66159407 | 56782 | 11: 66307327 | 56783 | 11: 66315381 |
| 56790 | 11: 66473480 | 56795 | 11: 66556801 | 56796 | 11: 66556834 | 56799 | 11: 66629962 | 56813 | 11: 67084838 |
| 56814 | 11: 67108786 | 56815 | 11: 67114080 | 56821 | 11: 67213238 | 56829 | 11: 67439320 | 56832 | 11: 67453087 |
| 56834 | 11: 67479748 | 56835 | 11: 67481439 | 56836 | 11: 67483125 | 56838 | 11: 67512057 | 56842 | 11: 67544307 |
| 56843 | 11: 67549232 | 56844 | 11: 67549363 | 56846 | 11: 67556514 | 56847 | 11: 67563313 | 56848 | 11: 67582936 |
| 56849 | 11: 67582999 | 56850 | 11: 67583014 | 56852 | 11: 67588734 | 56855 | 11: 67702445 | 56856 | 11: 67709590 |
| 56857 | 11: 67727467 | 56858 | 11: 67728102 | 56859 | 11: 67730113 | 56860 | 11: 67736102 | 56861 | 11: 67737073 |
| 56862 | 11: 67747241 | 56863 | 11: 67749685 | 56864 | 11: 67752237 | 56865 | 11: 67759745 | 56867 | 11: 67762854 |
| 56868 | 11: 67767430 | 56869 | 11: 67772466 | 56870 | 11: 67776969 | 56871 | 11: 67787768 | 56872 | 11: 67799383 |
| 56873 | 11: 67805662 | 56874 | 11: 67810598 | 56879 | 11: 67896985 | 56882 | 11: 67970890 | 56885 | 11: 68111905 |
| 56893 | 11: 68411391 | 56897 | 11: 68467750 | 56909 | 11: 68787390 | 56921 | 11: 69071509 | 56932 | 11: 69177619 |
| 56939 | 11: 69288577 | 56941 | 11: 69302814 | 56947 | 11: 69479974 | 56950 | 11: 69514045 | 56953 | 11: 69570750 |
| 56954 | 11: 69572243 | 56956 | 11: 69619243 | 56957 | 11: 69620020 | 56958 | 11: 69620110 | 56960 | 11: 69662827 |
| 56967 | 11: 69745012 | 56992 | 11: 70292588 | 56994 | 11: 70341851 | 56995 | 11: 70350355 | 57003 | 11: 70595154 |
| 57010 | 11: 70714155 | 57011 | 11: 70718274 | 57013 | 11: 70755284 | 57014 | 11: 70761156 | 57015 | 11: 70761179 |
| 57016 | 11: 70761872 | 57017 | 11: 70762170 | 57018 | 11: 70762689 | 57019 | 11: 70762700 | 57020 | 11: 70762702 |
| 57021 | 11: 70762781 | 57022 | 11: 70762862 | 57023 | 11: 70762868 | 57024 | 11: 70763022 | 57025 | 11: 70763076 |
| 57026 | 11: 70763145 | 57027 | 11: 70763553 | 57028 | 11: 70763715 | 57029 | 11: 70763906 | 57030 | 11: 70764185 |
| 57031 | 11: 70764539 | 57032 | 11: 70768163 | 57033 | 11: 70768281 | 57034 | 11: 70769360 | 57035 | 11: 70769405 |
| 57036 | 11: 70775819 | 57037 | 11: 70782767 | 57038 | 11: 70782825 | 57039 | 11: 70806849 | 57040 | 11: 70807701 |
| 57041 | 11: 70810781 | 57043 | 11: 70862638 | 57048 | 11: 71062901 | 57049 | 11: 71062934 | 57053 | 11: 71130818 |
| 57054 | 11: 71131623 | 57057 | 11: 71142342 | 57059 | 11: 71149248 | 57066 | 11: 71175312 | 57067 | 11: 71177865 |
| 57068 | 11: 71178203 | 57070 | 11: 71182683 | 57072 | 11: 71198214 | 57073 | 11: 71199013 | 57074 | 11: 71208751 |
| 57081 | 11: 71398446 | 57094 | 11: 71666080 | 57095 | 11: 71713947 | 57099 | 11: 71827640 | 57102 | 11: 71961107 |
| 57103 | 11: 71974375 | 57108 | 11: 72101578 | 57112 | 11: 72181248 | 57128 | 11: 72810370 | 57133 | 11: 72851670 |
| 57144 | 11: 72988812 | 57146 | 11: 73025557 | 57148 | 11: 73068186 | 57150 | 11: 73079672 | 57151 | 11: 73079767 |
| 57152 | 11: 73084466 | 57153 | 11: 73087461 | 57154 | 11: 73089558 | 57155 | 11: 73090095 | 57158 | 11: 73133312 |
| 57159 | 11: 73148896 | 57161 | 11: 73219332 | 57163 | 11: 73277111 | 57166 | 11: 73438358 | 57167 | 11: 73480475 |
| 57177 | 11: 73781192 | 57181 | 11: 73853326 | 57185 | 11: 73967619 | 57188 | 11: 74009513 | 57190 | 11: 74018063 |
| 57194 | 11: 74054528 | 57205 | 11: 74195149 | 57206 | 11: 74204674 | 57207 | 11: 74205573 | 57209 | 11: 74245610 |
| 57213 | 11: 74399369 | 57218 | 11: 74544795 | 57227 | 11: 74974483 | 57230 | 11: 75017890 | 57232 | 11: 75044406 |
| 57235 | 11: 75106218 | 57236 | 11: 75198521 | 57242 | 11: 75328149 | 57243 | 11: 75330425 | 57245 | 11: 75334844 |
| 57246 | 11: 75384323 | 57249 | 11: 75420509 | 57250 | 11: 75420581 | 57251 | 11: 75477129 | 57252 | 11: 75482256 |
| 57254 | 11: 75493333 | 57256 | 11: 75509869 | 57257 | 11: 75513821 | 57266 | 11: 75686867 | 57273 | 11: 75929380 |
| 57280 | 11: 76017351 | 57309 | 11: 76876371 | 57310 | 11: 76952531 | 57321 | 11: 77091873 | 57322 | 11: 77092007 |
| 57323 | 11: 77121176 | 57324 | 11: 77123614 | 57325 | 11: 77124254 | 57327 | 11: 77132776 | 57332 | 11: 77262392 |
| 57355 | 11: 77817486 | 57362 | 11: 77896861 | 57363 | 11: 77897300 | 57364 | 11: 77897480 | 57365 | 11: 77897481 |
| 57366 | 11: 77897524 | 57367 | 11: 77899208 | 57368 | 11: 77899796 | 57369 | 11: 77900345 | 57370 | 11: 77900437 |
| 57371 | 11: 77900488 | 57372 | 11: 77900522 | 57373 | 11: 77901876 | 57374 | 11: 77901997 | 57375 | 11: 77916472 |
| 57376 | 11: 77916653 | 57377 | 11: 77917793 | 57378 | 11: 77918799 | 57379 | 11: 77920101 | 57380 | 11: 77920618 |
| 57381 | 11: 77920740 | 57382 | 11: 77920832 | 57384 | 11: 77922179 | 57385 | 11: 77922588 | 57386 | 11: 77923067 |
| 57387 | 11: 77927350 | 57398 | 11: 78226756 | 57401 | 11: 78268974 | 57402 | 11: 78275050 | 57403 | 11: 78303645 |
| 57407 | 11: 78375724 | 57412 | 11: 78529857 | 57415 | 11: 78631628 | 57417 | 11: 78663074 | 57419 | 11: 78724728 |
| 57430 | 11: 78997006 | 57438 | 11: 79084041 | 57452 | 11: 79229476 | 57454 | 11: 79233862 | 57479 | 11: 79764049 |
| 57489 | 11: 80152318 | 57494 | 11: 80309473 | 57496 | 11: 80321806 | 57505 | 11: 80474151 | 57507 | 11: 80513212 |
| 57514 | 11: 80648310 | 57515 | 11: 80671943 | 57516 | 11: 80690342 | 57545 | 11: 81671201 | 57546 | 11: 81671227 |
| 57551 | 11: 81785970 | 57552 | 11: 81800692 | 57558 | 11: 81881809 | 57575 | 11: 82260811 | 57580 | 11: 82351733 |
| 57583 | 11: 82364482 | 57591 | 11: 82664554 | 57592 | 11: 82664600 | 57593 | 11: 82664609 | 57594 | 11: 82664725 |
| 57596 | 11: 82671761 | 57597 | 11: 82677475 | 57598 | 11: 82677728 | 57599 | 11: 82683785 | 57600 | 11: 82686842 |
| 57601 | 11: 82687013 | 57602 | 11: 82687072 | 57603 | 11: 82687666 | 57604 | 11: 82700336 | 57605 | 11: 82709413 |
| 57621 | 11: 83006401 | 57622 | 11: 83027036 | 57623 | 11: 83038638 | 57629 | 11: 83118274 | 57630 | 11: 83137062 |
| 57652 | 11: 83676352 | 57692 | 11: 84965273 | 57709 | 11: 85278395 | 57711 | 11: 85316120 | 57712 | 11: 85350978 |
| 57720 | 11: 85498225 | 57722 | 11: 85595081 | 57731 | 11: 85746062 | 57733 | 11: 85771451 | 57736 | 11: 85853743 |
| 57738 | 11: 85936758 | 57748 | 11: 86094641 | 57750 | 11: 86113594 | 57753 | 11: 86201573 | 57757 | 11: 86221979 |
| 57758 | 11: 86224683 | 57789 | 11: 87198869 | 57790 | 11: 87208683 | 57792 | 11: 87231125 | 57797 | 11: 87357375 |
| 57798 | 11: 87380616 | 57803 | 11: 87492469 | 57804 | 11: 87494268 | 57805 | 11: 87495906 | 57807 | 11: 87520617 |
| 57808 | 11: 87530939 | 57812 | 11: 87650800 | 57815 | 11: 87710317 | 57821 | 11: 87813732 | 57823 | 11: 87882552 |
| 57829 | 11: 87970096 | 57830 | 11: 87972654 | 57831 | 11: 87972763 | 57832 | 11: 87975372 | 57833 | 11: 87980700 |
| 57834 | 11: 87982062 | 57835 | 11: 87982864 | 57836 | 11: 87986573 | 57837 | 11: 87987392 | 57838 | 11: 87991576 |
| 57839 | 11: 87992353 | 57840 | 11: 87994077 | 57841 | 11: 87994761 | 57842 | 11: 87995976 | 57843 | 11: 87996439 |
| 57844 | 11: 87997041 | 57845 | 11: 87997518 | 57846 | 11: 87998024 | 57848 | 11: 88000459 | 57849 | 11: 88001097 |
| 57850 | 11: 88002138 | 57851 | 11: 88003290 | 57852 | 11: 88006051 | 57853 | 11: 88006580 | 57854 | 11: 88007139 |
| 57855 | 11: 88008201 | 57857 | 11: 88009081 | 57858 | 11: 88009883 | 57859 | 11: 88011349 | 57860 | 11: 88013047 |
| 57862 | 11: 88020282 | 57863 | 11: 88021880 | 57864 | 11: 88030641 | 57866 | 11: 88045066 | 57873 | 11: 88105264 |
| 57885 | 11: 88479108 | 57890 | 11: 88733341 | 57897 | 11: 88922785 | 57898 | 11: 88922827 | 57899 | 11: 88922860 |
| 57902 | 11: 88976021 | 57904 | 11: 88994635 | 57909 | 11: 89132552 | 57910 | 11: 89133642 | 57911 | 11: 89152187 |
| 57912 | 11: 89213759 | 57919 | 11: 89375784 | 57920 | 11: 89387726 | 57929 | 11: 89561970 | 57940 | 11: 89843767 |
| 57960 | 11: 90408075 | 57965 | 11: 90443507 | 57970 | 11: 90503652 | 57977 | 11: 90744044 | 57979 | 11: 90785269 |
| 57980 | 11: 90787434 | 57984 | 11: 90906061 | 57989 | 11: 90982703 | 57991 | 11: 91054916 | 57994 | 11: 91084013 |
| 58002 | 11: 91266370 | 58006 | 11: 91343800 | 58061 | 11: 92402829 | 58062 | 11: 92454234 | 58063 | 11: 92473780 |
| 58064 | 11: 92476182 | 58073 | 11: 92661797 | 58074 | 11: 92674766 | 58077 | 11: 92715331 | 58084 | 11: 92833095 |
| 58095 | 11: 93210615 | 58098 | 11: 93223406 | 58116 | 11: 93629686 | 58119 | 11: 93727304 | 58126 | 11: 93972515 |
| 58127 | 11: 93973132 | 58128 | 11: 93973249 | 58129 | 11: 93973432 | 58131 | 11: 94067508 | 58132 | 11: 94076855 |
| 58137 | 11: 94178079 | 58140 | 11: 94211102 | 58144 | 11: 94339459 | 58148 | 11: 94453289 | 58151 | 11: 94500564 |
| 58152 | 11: 94531181 | 58164 | 11: 94880948 | 58173 | 11: 95280882 | 58177 | 11: 95315895 | 58178 | 11: 95322263 |
| 58181 | 11: 95425616 | 58190 | 11: 95586651 | 58193 | 11: 95692063 | 58201 | 11: 95840093 | 58207 | 11: 96117932 |
| 58209 | 11: 96142116 | 58213 | 11: 96261764 | 58215 | 11: 96285921 | 58216 | 11: 96322123 | 58217 | 11: 96322228 |
| 58218 | 11: 96338458 | 58230 | 11: 96820150 | 58245 | 11: 97196967 | 58248 | 11: 97225356 | 58251 | 11: 97254785 |
| 58253 | 11: 97286893 | 58276 | 11: 97843712 | 58277 | 11: 97843818 | 58278 | 11: 97883599 | 58279 | 11: 97892319 |
| 58289 | 11: 98162222 | 58290 | 11: 98162477 | 58291 | 11: 98173675 | 58292 | 11: 98179996 | 58293 | 11: 98189444 |
| 58294 | 11: 98191816 | 58295 | 11: 98193416 | 58298 | 11: 98222190 | 58304 | 11: 98396073 | 58313 | 11: 98726635 |
| 58316 | 11: 98813314 | 58321 | 11: 98866430 | 58327 | 11: 98921444 | 58328 | 11: 98921481 | 58331 | 11: 98929180 |
| 58332 | 11: 98929534 | 58333 | 11: 98931991 | 58334 | 11: 98932587 | 58335 | 11: 98951509 | 58337 | 11: 98980770 |
| 58338 | 11: 99034896 | 58339 | 11: 99114771 | 58342 | 11: 99223633 | 58343 | 11: 99225289 | 58346 | 11: 99292200 |
| 58347 | 11: 99366886 | 58348 | 11: 99391843 | 58349 | 11: 99395118 | 58350 | 11: 99395609 | 58351 | 11: 99396169 |
| 58352 | 11: 99397143 | 58353 | 11: 99398436 | 58354 | 11: 99399218 | 58355 | 11: 99400709 | 58356 | 11: 99401571 |
| 58357 | 11: 99402294 | 58358 | 11: 99403416 | 58359 | 11: 99409144 | 58360 | 11: 99424233 | 58361 | 11: 99434036 |
| 58362 | 11: 99444575 | 58368 | 11: 99668885 | 58378 | 11: 99888002 | 58382 | 11: 99923257 | 58383 | 11: 99932292 |
| 58384 | 11: 99932294 | 58392 | 11: 10002865 | 58402 | 11: 10020523 | 58404 | 11: 10023875 | 58405 | 11: 10024462 |
| 58408 | 11: 10034694 | 58413 | 11: 10046250 | 58421 | 11: 10065868 | 58423 | 11: 10069663 | 58432 | 11: 10089413 |
| 58433 | 11: 10089414 | 58434 | 11: 10089418 | 58435 | 11: 10089420 | 58436 | 11: 10089422 | 58437 | 11: 10089423 |
| 58438 | 11: 10089584 | 58439 | 11: 10089862 | 58440 | 11: 10089910 | 58441 | 11: 10090030 | 58442 | 11: 10090083 |
| 58443 | 11: 10090139 | 58444 | 11: 10090298 | 58445 | 11: 10090301 | 58446 | 11: 10090303 | 58447 | 11: 10090310 |
| 58448 | 11: 10090437 | 58449 | 11: 10090548 | 58450 | 11: 10090552 | 58451 | 11: 10090559 | 58452 | 11: 10092003 |
| 58453 | 11: 10092018 | 58455 | 11: 10092948 | 58456 | 11: 10092991 | 58465 | 11: 10121113 | 58469 | 11: 10139576 |
| 58481 | 11: 10176766 | 58482 | 11: 10179134 | 58490 | 11: 10195025 | 58498 | 11: 10216529 | 58503 | 11: 10235669 |
| 58505 | 11: 10236915 | 58507 | 11: 10245112 | 58508 | 11: 10245553 | 58509 | 11: 10245561 | 58510 | 11: 10245564 |
| 58512 | 11: 10248040 | 58520 | 11: 10260314 | 58521 | 11: 10260318 | 58527 | 11: 10273417 | 58534 | 11: 10280891 |
| 58535 | 11: 10282294 | 58537 | 11: 10284704 | 58539 | 11: 10287469 | 58540 | 11: 10287825 | 58541 | 11: 10287894 |
| 58542 | 11: 10288318 | 58543 | 11: 10288773 | 58544 | 11: 10289321 | 58545 | 11: 10289432 | 58546 | 11: 10289512 |
| 58547 | 11: 10289609 | 58548 | 11: 10289667 | 58549 | 11: 10289886 | 58550 | 11: 10289974 | 58551 | 11: 10290070 |
| 58552 | 11: 10290097 | 58553 | 11: 10290321 | 58555 | 11: 10293724 | 58561 | 11: 10303129 | 58563 | 11: 10311374 |
| 58565 | 11: 10312784 | 58570 | 11: 10325911 | 58571 | 11: 10325913 | 58572 | 11: 10325914 | 58573 | 11: 10325923 |
| 58574 | 11: 10327300 | 58580 | 11: 10337533 | 58582 | 11: 10338027 | 58586 | 11: 10342689 | 58599 | 11: 10376543 |
| 58603 | 11: 10389419 | 58610 | 11: 10416326 | 58611 | 11: 10416332 | 58614 | 11: 10418835 | 58616 | 11: 10420735 |
| 58621 | 11: 10424706 | 58626 | 11: 10440574 | 58641 | 11: 10466512 | 58647 | 11: 10471723 | 58648 | 11: 10472240 |
| 58664 | 11: 10499756 | 58666 | 11: 10508228 | 58669 | 11: 10514921 | 58675 | 11: 10524663 | 58676 | 11: 10526769 |
| 58678 | 11: 10536852 | 58679 | 11: 10536862 | 58682 | 11: 10549667 | 58683 | 11: 10556546 | 58684 | 11: 10556548 |
| 58685 | 11: 10556557 | 58686 | 11: 10558647 | 58687 | 11: 10559874 | 58688 | 11: 10560239 | 58689 | 11: 10560299 |
| 58690 | 11: 10560406 | 58691 | 11: 10560652 | 58692 | 11: 10560775 | 58693 | 11: 10560879 | 58694 | 11: 10560986 |
| 58695 | 11: 10561194 | 58696 | 11: 10561355 | 58697 | 11: 10561585 | 58698 | 11: 10561748 | 58699 | 11: 10561828 |
| 58700 | 11: 10562085 | 58701 | 11: 10562158 | 58702 | 11: 10562413 | 58703 | 11: 10562929 | 58704 | 11: 10567074 |
| 58705 | 11: 10569241 | 58707 | 11: 10576176 | 58712 | 11: 10606865 | 58713 | 11: 10608376 | 58715 | 11: 10621054 |
| 58717 | 11: 10634575 | 58719 | 11: 10644949 | 58722 | 11: 10657444 | 58723 | 11: 10657459 | 58724 | 11: 10657485 |
| 58725 | 11: 10661179 | 58726 | 11: 10661476 | 58727 | 11: 10662633 | 58740 | 12: 317570 | 58748 | 12: 542288 |
| 58757 | 12: 877215 | 58797 | 12: 1923479 | 58805 | 12: 2038461 | 58813 | 12: 2256922 | 58819 | 12: 2323582 |
| 58825 | 12: 2448377 | 58826 | 12: 2448435 | 58827 | 12: 2452533 | 58828 | 12: 2452685 | 58829 | 12: 2509817 |
| 58836 | 12: 2603168 | 58839 | 12: 2644727 | 58840 | 12: 2644772 | 58841 | 12: 2644793 | 58844 | 12: 2731342 |
| 58846 | 12: 2771593 | 58923 | 12: 4734740 | 58937 | 12: 5055337 | 59006 | 12: 7477665 | 59040 | 12: 8789258 |
| 59041 | 12: 8796877 | 59053 | 12: 9132925 | 59062 | 12: 9382289 | 59066 | 12: 9448144 | 59077 | 12: 9708609 |
| 59078 | 12: 9708626 | 59093 | 12: 10135415 | 59095 | 12: 10160043 | 59114 | 12: 10462955 | 59122 | 12: 10621365 |
| 59123 | 12: 10622131 | 59124 | 12: 10631694 | 59125 | 12: 10631700 | 59134 | 12: 10781199 | 59152 | 12: 11193032 |
| 59170 | 12: 11622983 | 59171 | 12: 11641143 | 59173 | 12: 11670644 | 59175 | 12: 11684828 | 59186 | 12: 11878651 |
| 59189 | 12: 11933919 | 59193 | 12: 12026687 | 59195 | 12: 12066263 | 59196 | 12: 12069687 | 59197 | 12: 12087862 |
| 59198 | 12: 12123346 | 59206 | 12: 12322089 | 59210 | 12: 12404824 | 59219 | 12: 12760273 | 59224 | 12: 12974557 |
| 59231 | 12: 13161754 | 59239 | 12: 13388932 | 59241 | 12: 13411406 | 59242 | 12: 13473520 | 59243 | 12: 13518818 |
| 59246 | 12: 13578439 | 59247 | 12: 13591408 | 59250 | 12: 13656437 | 59261 | 12: 13824057 | 59268 | 12: 13927173 |
| 59280 | 12: 14131140 | 59281 | 12: 14132377 | 59283 | 12: 14157283 | 59285 | 12: 14211580 | 59286 | 12: 14211590 |
| 59287 | 12: 14211610 | 59288 | 12: 14240924 | 59292 | 12: 14361824 | 59293 | 12: 14372218 | 59294 | 12: 14388145 |
| 59295 | 12: 14399933 | 59299 | 12: 14456910 | 59302 | 12: 14520853 | 59306 | 12: 14595181 | 59311 | 12: 14716329 |
| 59319 | 12: 14988572 | 59321 | 12: 15005389 | 59324 | 12: 15101552 | 59333 | 12: 15266724 | 59335 | 12: 15281976 |
| 59344 | 12: 15465428 | 59348 | 12: 15534714 | 59350 | 12: 15552667 | 59352 | 12: 15567714 | 59353 | 12: 15577383 |
| 59355 | 12: 15618825 | 59360 | 12: 15729746 | 59363 | 12: 15770061 | 59366 | 12: 15810335 | 59373 | 12: 15938324 |
| 59380 | 12: 16125597 | 59381 | 12: 16136554 | 59386 | 12: 16274543 | 59390 | 12: 16346416 | 59394 | 12: 16413245 |
| 59410 | 12: 16793613 | 59414 | 12: 16900417 | 59423 | 12: 17162528 | 59425 | 12: 17199573 | 59430 | 12: 17324217 |
| 59435 | 12: 17368614 | 59440 | 12: 17470036 | 59453 | 12: 17926477 | 59459 | 12: 18040185 | 59468 | 12: 18229565 |
| 59469 | 12: 18253619 | 59474 | 12: 18390032 | 59475 | 12: 18391314 | 59486 | 12: 18697557 | 59492 | 12: 18898137 |
| 59496 | 12: 18919039 | 59507 | 12: 19071459 | 59509 | 12: 19100832 | 59517 | 12: 19186475 | 59519 | 12: 19221055 |
| 59520 | 12: 19233240 | 59522 | 12: 19351299 | 59523 | 12: 19367974 | 59524 | 12: 19367977 | 59528 | 12: 19460445 |
| 59545 | 12: 19931195 | 59550 | 12: 20111148 | 59551 | 12: 20111169 | 59578 | 12: 20706397 | 59582 | 12: 20914485 |
| 59588 | 12: 21042616 | 59591 | 12: 21070776 | 59597 | 12: 21212930 | 59600 | 12: 21309091 | 59602 | 12: 21368915 |
| 59613 | 12: 21811288 | 59617 | 12: 21924603 | 59618 | 12: 21936752 | 59620 | 12: 21945571 | 59621 | 12: 21969243 |
| 59627 | 12: 22146118 | 59631 | 12: 22263714 | 59642 | 12: 22573355 | 59643 | 12: 22576349 | 59647 | 12: 22662928 |
| 59653 | 12: 22821830 | 59654 | 12: 22834458 | 59663 | 12: 23060365 | 59667 | 12: 23201662 | 59671 | 12: 23249052 |
| 59677 | 12: 23371031 | 59680 | 12: 23439101 | 59696 | 12: 23931995 | 59703 | 12: 24076421 | 59710 | 12: 24256758 |
| 59716 | 12: 24331867 | 59721 | 12: 24471307 | 59733 | 12: 24731505 | 59734 | 12: 24748284 | 59737 | 12: 24838866 |
| 59741 | 12: 24922215 | 59742 | 12: 24925957 | 59747 | 12: 25022781 | 59749 | 12: 25034947 | 59754 | 12: 25118490 |
| 59766 | 12: 25470724 | 59767 | 12: 25498467 | 59769 | 12: 25589790 | 59777 | 12: 25724323 | 59798 | 12: 26442722 |
| 59805 | 12: 26653552 | 59808 | 12: 26751983 | 59814 | 12: 26874891 | 59820 | 12: 26979789 | 59821 | 12: 26986008 |
| 59830 | 12: 27189356 | 59841 | 12: 27380634 | 59848 | 12: 27503259 | 59850 | 12: 27528591 | 59854 | 12: 27721180 |
| 59855 | 12: 27723132 | 59856 | 12: 27723229 | 59861 | 12: 27998192 | 59862 | 12: 28002030 | 59878 | 12: 28526148 |
| 59879 | 12: 28587383 | 59881 | 12: 28621183 | 59893 | 12: 28989343 | 59913 | 12: 29539899 | 59934 | 12: 30141838 |
| 59944 | 12: 30381579 | 59945 | 12: 30411312 | 59947 | 12: 30444618 | 59949 | 12: 30463422 | 59969 | 12: 30909596 |
| 59971 | 12: 30979325 | 59973 | 12: 30983862 | 59974 | 12: 30991739 | 59977 | 12: 31053899 | 59979 | 12: 31059835 |
| 59980 | 12: 31115741 | 59984 | 12: 31247300 | 59985 | 12: 31247421 | 59986 | 12: 31247454 | 59987 | 12: 31247486 |
| 59998 | 12: 31454833 | 60007 | 12: 31767190 | 60011 | 12: 31790778 | 60012 | 12: 31797116 | 60018 | 12: 31950273 |
| 60023 | 12: 32162560 | 60028 | 12: 32295933 | 60029 | 12: 32338936 | 60032 | 12: 32379592 | 60033 | 12: 32387590 |
| 60034 | 12: 32391405 | 60037 | 12: 32450230 | 60038 | 12: 32450232 | 60040 | 12: 32487563 | 60058 | 12: 33054434 |
| 60061 | 12: 33082346 | 60062 | 12: 33085573 | 60066 | 12: 33139330 | 60075 | 12: 33340233 | 60086 | 12: 33674224 |
| 60095 | 12: 33970418 | 60096 | 12: 34054782 | 60097 | 12: 34068458 | 60109 | 12: 34500254 | 60114 | 12: 34681917 |
| 60126 | 12: 34900938 | 60133 | 12: 35115665 | 60140 | 12: 35310855 | 60150 | 12: 35346851 | 60164 | 12: 35747252 |
| 60173 | 12: 36216803 | 60175 | 12: 36341507 | 60176 | 12: 36361684 | 60178 | 12: 36421378 | 60179 | 12: 36443014 |
| 60181 | 12: 36474506 | 60182 | 12: 36483162 | 60183 | 12: 36486137 | 60184 | 12: 36504213 | 60189 | 12: 36706435 |
| 60191 | 12: 36753159 | 60195 | 12: 36952382 | 60196 | 12: 36952416 | 60210 | 12: 37329971 | 60211 | 12: 37330062 |
| 60212 | 12: 37330099 | 60216 | 12: 37420551 | 60256 | 12: 39391630 | 60260 | 12: 39553608 | 60267 | 12: 39684127 |
| 60270 | 12: 39776454 | 60279 | 12: 40049870 | 60281 | 12: 40141911 | 60311 | 12: 41076939 | 60329 | 12: 41861946 |
| 60332 | 12: 41912763 | 60402 | 12: 44937336 | 60404 | 12: 44979318 | 60422 | 12: 45617161 | 60442 | 12: 46329415 |
| 60445 | 12: 46430991 | 60466 | 12: 46961986 | 60470 | 12: 47018801 | 60474 | 12: 47110627 | 60484 | 12: 47309976 |
| 60487 | 12: 47407515 | 60508 | 12: 48047961 | 60510 | 12: 48150915 | 60538 | 12: 48960299 | 60549 | 12: 49199679 |
| 60552 | 12: 49236831 | 60566 | 12: 49561422 | 60571 | 12: 49723456 | 60574 | 12: 49789492 | 60579 | 12: 49873319 |
| 60580 | 12: 49881472 | 60584 | 12: 49919596 | 60600 | 12: 50189626 | 60609 | 12: 50384404 | 60611 | 12: 50424103 |
| 60612 | 12: 50439814 | 60614 | 12: 50497707 | 60615 | 12: 50517145 | 60616 | 12: 50525554 | 60617 | 12: 50530876 |
| 60619 | 12: 50579721 | 60628 | 12: 50806532 | 60630 | 12: 50840958 | 60632 | 12: 50869818 | 60633 | 12: 50871829 |
| 60660 | 12: 51551817 | 60661 | 12: 51626384 | 60679 | 12: 52103992 | 60687 | 12: 52262646 | 60692 | 12: 52346386 |
| 60702 | 12: 52660295 | 60710 | 12: 52806268 | 60727 | 12: 53053258 | 60729 | 12: 53061165 | 60738 | 12: 53338165 |
| 60739 | 12: 53339046 | 60740 | 12: 53339054 | 60749 | 12: 53523285 | 60750 | 12: 53553256 | 60752 | 12: 53587669 |
| 60755 | 12: 53627763 | 60756 | 12: 53640047 | 60763 | 12: 53801258 | 60781 | 12: 54131348 | 60782 | 12: 54153871 |
| 60797 | 12: 54494995 | 60803 | 12: 54630943 | 60804 | 12: 54634286 | 60810 | 12: 54736016 | 60813 | 12: 54833257 |
| 60815 | 12: 54927343 | 60819 | 12: 55024877 | 60821 | 12: 55068443 | 60848 | 12: 55943119 | 60851 | 12: 56039366 |
| 60880 | 12: 56976868 | 60882 | 12: 57050432 | 60904 | 12: 57798387 | 60905 | 12: 57798425 | 60912 | 12: 57952881 |
| 60914 | 12: 58028126 | 60929 | 12: 58672694 | 60957 | 12: 59417372 | 60969 | 12: 59853749 | 60978 | 12: 60236147 |
| 60990 | 12: 60736444 | 61018 | 12: 61855777 | 61024 | 12: 62071314 | 61028 | 12: 62095959 | 61031 | 12: 62215048 |
| 61034 | 12: 62291744 | 61035 | 12: 62297558 | 61051 | 12: 62665927 | 61053 | 12: 62697756 | 61065 | 12: 63053238 |
| 61080 | 12: 63589109 | 61085 | 12: 63730818 | 61089 | 12: 63774437 | 61094 | 12: 63920843 | 61100 | 12: 64086485 |
| 61112 | 12: 64454937 | 61126 | 12: 64874897 | 61127 | 12: 64896393 | 61148 | 12: 65376279 | 61151 | 12: 65433100 |
| 61156 | 12: 65616303 | 61168 | 12: 66123917 | 61170 | 12: 66204351 | 61175 | 12: 66340744 | 61199 | 12: 66896772 |
| 61206 | 12: 67184352 | 61207 | 12: 67228835 | 61210 | 12: 67289617 | 61216 | 12: 67417050 | 61231 | 12: 67757020 |
| 61237 | 12: 67889485 | 61252 | 12: 68144427 | 61255 | 12: 68171444 | 61256 | 12: 68171491 | 61262 | 12: 68316664 |
| 61274 | 12: 68605490 | 61280 | 12: 68760089 | 61283 | 12: 68784416 | 61289 | 12: 68861830 | 61321 | 12: 69512279 |
| 61322 | 12: 69512294 | 61323 | 12: 69516800 | 61331 | 12: 69577020 | 61338 | 12: 69681798 | 61348 | 12: 70147062 |
| 61367 | 12: 71314524 | 61383 | 12: 71639973 | 61386 | 12: 71790421 | 61390 | 12: 71842435 | 61408 | 12: 72931638 |
| 61414 | 12: 73012734 | 61421 | 12: 73132371 | 61422 | 12: 73154971 | 61427 | 12: 73317927 | 61428 | 12: 73324264 |
| 61441 | 12: 73504167 | 61443 | 12: 73515714 | 61444 | 12: 73515717 | 61445 | 12: 73515812 | 61454 | 12: 73622472 |
| 61455 | 12: 73637550 | 61462 | 12: 73750586 | 61469 | 12: 73899680 | 61475 | 12: 74054821 | 61486 | 12: 74328198 |
| 61495 | 12: 74492364 | 61498 | 12: 74505038 | 61500 | 12: 74526665 | 61517 | 12: 74949213 | 61530 | 12: 75243236 |
| 61535 | 12: 75318404 | 61538 | 12: 75375959 | 61542 | 12: 75403639 | 61543 | 12: 75471804 | 61558 | 12: 75839630 |
| 61567 | 12: 76074190 | 61569 | 12: 76110122 | 61581 | 12: 76477335 | 61587 | 12: 76644169 | 61588 | 12: 76649683 |
| 61605 | 12: 77139238 | 61609 | 12: 77240177 | 61627 | 12: 77604409 | 61632 | 12: 77695108 | 61641 | 12: 77891817 |
| 61644 | 12: 77992115 | 61647 | 12: 78041886 | 61655 | 12: 78246588 | 61669 | 12: 78577349 | 61691 | 12: 79095439 |
| 61692 | 12: 79098661 | 61693 | 12: 79098791 | 61694 | 12: 79099207 | 61695 | 12: 79099246 | 61696 | 12: 79104872 |
| 61710 | 12: 79403358 | 61715 | 12: 79476337 | 61727 | 12: 79736085 | 61740 | 12: 79970178 | 61756 | 12: 80319360 |
| 61775 | 12: 80804698 | 61790 | 12: 81214960 | 61791 | 12: 81215027 | 61803 | 12: 81507963 | 61804 | 12: 81508005 |
| 61819 | 12: 81781296 | 61820 | 12: 81840070 | 61824 | 12: 81902349 | 61840 | 12: 82263825 | 61845 | 12: 82445861 |
| 61848 | 12: 82510342 | 61877 | 12: 83071777 | 61882 | 12: 83242885 | 61891 | 12: 83362092 | 61907 | 12: 83699595 |
| 61915 | 12: 83925298 | 61920 | 12: 84006533 | 61926 | 12: 84137136 | 61945 | 12: 84691330 | 61946 | 12: 84709160 |
| 61948 | 12: 84744945 | 61949 | 12: 84744950 | 61955 | 12: 84883821 | 61960 | 12: 84929565 | 61966 | 12: 84993056 |
| 61969 | 12: 85038979 | 61970 | 12: 85051267 | 61973 | 12: 85122781 | 61978 | 12: 85226982 | 61979 | 12: 85230036 |
| 61981 | 12: 85245683 | 61983 | 12: 85268459 | 61984 | 12: 85293670 | 61986 | 12: 85321955 | 61988 | 12: 85328690 |
| 61990 | 12: 85352315 | 61991 | 12: 85354082 | 61994 | 12: 85362756 | 61995 | 12: 85375388 | 61997 | 12: 85396298 |
| 62000 | 12: 85462507 | 62007 | 12: 85632578 | 62013 | 12: 85790348 | 62014 | 12: 85790349 | 62035 | 12: 86313847 |
| 62038 | 12: 86341413 | 62043 | 12: 86507763 | 62045 | 12: 86516372 | 62046 | 12: 86516421 | 62048 | 12: 86519761 |
| 62049 | 12: 86565634 | 52051 | 12: 86656646 | 62052 | 12: 86687289 | 62053 | 12: 86688052 | 62056 | 12: 86734179 |
| 62058 | 12: 86744155 | 62059 | 12: 86764063 | 62062 | 12: 86796745 | 62064 | 12: 86824616 | 62068 | 12: 86895391 |
| 62069 | 12: 86895562 | 62070 | 12: 86897090 | 62071 | 12: 86897178 | 62072 | 12: 86901814 | 62073 | 12: 86901834 |
| 62074 | 12: 86901846 | 62075 | 12: 86901945 | 62076 | 12: 86902939 | 62077 | 12: 86902946 | 62078 | 12: 86907527 |
| 62079 | 12: 86907549 | 62080 | 12: 86908054 | 62082 | 12: 86924401 | 62085 | 12: 86976449 | 62086 | 12: 86988484 |
| 62089 | 12: 87019731 | 62090 | 12: 87024506 | 62095 | 12: 87094553 | 62109 | 13: 837989 | 62110 | 13: 859954 |
| 62115 | 13: 877219 | 62122 | 13: 1025761 | 62126 | 13: 1174223 | 62127 | 13: 1184941 | 62138 | 13: 1435091 |
| 62143 | 13: 1589719 | 62145 | 13: 1615667 | 62154 | 13: 1741335 | 62155 | 13: 1754933 | 62165 | 13: 2014401 |
| 62169 | 13: 2079335 | 62174 | 13: 2108318 | 62176 | 13: 2133989 | 62181 | 13: 2232512 | 62188 | 13: 2272732 |
| 62193 | 13: 2391867 | 62194 | 13: 2394109 | 62195 | 13: 2400920 | 62196 | 13: 2402378 | 62200 | 13: 2507817 |
| 62203 | 13: 2551762 | 62208 | 13: 2625076 | 62209 | 13: 2632788 | 62238 | 13: 3279851 | 62250 | 13: 3588425 |
| 62254 | 13: 3701619 | 62259 | 13: 3860445 | 62265 | 13: 3974443 | 62270 | 13: 4084139 | 62271 | 13: 4084171 |
| 62272 | 13: 4084181 | 62276 | 13: 4221428 | 62278 | 13: 4302438 | 62291 | 13: 4792202 | 62292 | 13: 4793338 |
| 62312 | 13: 5324308 | 62314 | 13: 5360261 | 62325 | 13: 5525194 | 62326 | 13: 5525225 | 62327 | 13: 5525250 |
| 62341 | 13: 5742073 | 62356 | 13: 6112364 | 62377 | 13: 6686807 | 62379 | 13: 6694933 | 62384 | 13: 6837817 |
| 62392 | 13: 7036389 | 62399 | 13: 7224958 | 62406 | 13: 7441639 | 62412 | 13: 7553513 | 62425 | 13: 7848557 |
| 62441 | 13: 8487756 | 62465 | 13: 8980654 | 62477 | 13: 9357735 | 62486 | 13: 9550333 | 62487 | 13: 9576478 |
| 62496 | 13: 9729764 | 62497 | 13: 9729772 | 62512 | 13: 10026414 | 62514 | 13: 10070381 | 62522 | 13: 10306871 |
| 62523 | 13: 10395756 | 62529 | 13: 10478767 | 62533 | 13: 10517565 | 62542 | 13: 10720404 | 62543 | 13: 10724228 |
| 62547 | 13: 10815809 | 62549 | 13: 10956279 | 62550 | 13: 10956346 | 62551 | 13: 10956354 | 62552 | 13: 10956355 |
| 62559 | 13: 11536433 | 62561 | 13: 11561251 | 62562 | 13: 11598409 | 62566 | 13: 11792199 | 62568 | 13: 11904569 |
| 62581 | 13: 12520500 | 62584 | 13: 12616676 | 62585 | 13: 12669714 | 62593 | 13: 12825549 | 62597 | 13: 12901657 |
| 62601 | 13: 12957260 | 62604 | 13: 13014675 | 62606 | 13: 13050532 | 62609 | 13: 13134686 | 62620 | 13: 13394218 |
| 62624 | 13: 13416442 | 62629 | 13: 13525172 | 62633 | 13: 13724404 | 62648 | 13: 14180541 | 62709 | 13: 15922152 |
| 62710 | 13: 15926948 | 62713 | 13: 16113457 | 62714 | 13: 16141551 | 62715 | 13: 16161619 | 62716 | 13: 16198035 |
| 62727 | 13: 16479721 | 62752 | 13: 16821257 | 62753 | 13: 16838893 | 62758 | 13: 16979210 | 62766 | 13: 17176766 |
| 62775 | 13: 17311025 | 62776 | 13: 17311026 | 62777 | 13: 17311048 | 62778 | 13: 17311402 | 62779 | 13: 17313556 |
| 62780 | 13: 17321384 | 62781 | 13: 17327212 | 62784 | 13: 17371815 | 62791 | 13: 17532109 | 62792 | 13: 17537709 |
| 62793 | 13: 17537777 | 62794 | 13: 17537795 | 62795 | 13: 17540260 | 62796 | 13: 17540346 | 62797 | 13: 17543085 |
| 62800 | 13: 17568204 | 62804 | 13: 17707891 | 62805 | 13: 17739721 | 62807 | 13: 17780066 | 62809 | 13: 17793717 |
| 62810 | 13: 17796108 | 62811 | 13: 17797006 | 62812 | 13: 17797028 | 62813 | 13: 17797078 | 62814 | 13: 17800892 |
| 62819 | 13: 17959881 | 62821 | 13: 18000186 | 62822 | 13: 18000196 | 62823 | 13: 18001014 | 62824 | 13: 18001496 |
| 62825 | 13: 18001505 | 62826 | 13: 18001601 | 62828 | 13: 18117994 | 62831 | 13: 18160335 | 62843 | 13: 18626070 |
| 62847 | 13: 18666175 | 62853 | 13: 18721208 | 62881 | 13: 18755905 | 62883 | 13: 18792716 | 62884 | 13: 18792825 |
| 62885 | 13: 18792850 | 62889 | 13: 18828811 | 62891 | 13: 18892498 | 62892 | 13: 18897253 | 62894 | 13: 19005851 |
| 62895 | 13: 19009052 | 62896 | 13: 19009093 | 62897 | 13: 19009113 | 62902 | 13: 19106619 | 62911 | 13: 19331702 |
| 62917 | 13: 19457766 | 62919 | 13: 19497053 | 62920 | 13: 19497075 | 62921 | 13: 19497132 | 62922 | 13: 19497137 |
| 62923 | 13: 19539755 | 62924 | 13: 19567930 | 62926 | 13: 19588565 | 62929 | 13: 19655690 | 62930 | 13: 19688739 |
| 62933 | 13: 19726581 | 62935 | 13: 19733380 | 62941 | 13: 19822361 | 62942 | 13: 19829540 | 62948 | 13: 19908550 |
| 62950 | 13: 19929408 | 62951 | 13: 19985595 | 62966 | 13: 20376052 | 62973 | 13: 20455853 | 62974 | 13: 20455858 |
| 62975 | 13: 20455892 | 62976 | 13: 20455934 | 62977 | 13: 20455941 | 62986 | 13: 20690765 | 62989 | 13: 20711502 |
| 63003 | 13: 21079068 | 63004 | 13: 21080622 | 63005 | 13: 21080632 | 63006 | 13: 21080639 | 63007 | 13: 21080653 |
| 63008 | 13: 21080676 | 63009 | 13: 21080695 | 63015 | 13: 21196496 | 63021 | 13: 21338503 | 63025 | 13: 21463530 |
| 63033 | 13: 21602178 | 63034 | 13: 21608876 | 63036 | 13: 21617194 | 63037 | 13: 21669388 | 63038 | 13: 21673325 |
| 63046 | 13: 21871770 | 63053 | 13: 22005224 | 63067 | 13: 22364650 | 63069 | 13: 22400352 | 63075 | 13: 22556435 |
| 63083 | 13: 22843425 | 63085 | 13: 22943752 | 63094 | 13: 23364320 | 63095 | 13: 23428241 | 63100 | 13: 23590170 |
| 63107 | 13: 23799970 | 63113 | 13: 23993051 | 63122 | 13: 24204881 | 63125 | 13: 24378395 | 63128 | 13: 24432283 |
| 63129 | 13: 24464855 | 63138 | 13: 24550804 | 63144 | 13: 24721448 | 63148 | 13: 24785943 | 63152 | 13: 24889747 |
| 63157 | 13: 25026767 | 63162 | 13: 25139101 | 63163 | 13: 25157343 | 63164 | 13: 25169602 | 63171 | 13: 25314039 |
| 63176 | 13: 25373782 | 63177 | 13: 25408450 | 63178 | 13: 25410196 | 63185 | 13: 25553780 | 63186 | 13: 25556386 |
| 63197 | 13: 25892715 | 63200 | 13: 25911303 | 63201 | 13: 25911339 | 63202 | 13: 25911366 | 63204 | 13: 25958875 |
| 63207 | 13: 26100997 | 63216 | 13: 26381171 | 63217 | 13: 26385197 | 63218 | 13: 26403532 | 63228 | 13: 26709761 |
| 63229 | 13: 26798911 | 63246 | 13: 27211452 | 63249 | 13: 27252426 | 63255 | 13: 27371552 | 63259 | 13: 27465175 |
| 63260 | 13: 27465215 | 63261 | 13: 27465241 | 63262 | 13: 27465291 | 63264 | 13: 27517101 | 63267 | 13: 27585269 |
| 63269 | 13: 27634615 | 63274 | 13: 27760115 | 63275 | 13: 27831742 | 63276 | 13: 27837915 | 63277 | 13: 27868901 |
| 63278 | 13: 27871914 | 63279 | 13: 27897181 | 63280 | 13: 27902374 | 63281 | 13: 27904185 | 63293 | 13: 28285297 |
| 63298 | 13: 28416787 | 63306 | 13: 28593403 | 63307 | 13: 28631602 | 63309 | 13: 28668818 | 63317 | 13: 28768035 |
| 63322 | 13: 28950265 | 63330 | 13: 29182643 | 63337 | 13: 29330098 | 63339 | 13: 29336139 | 63350 | 13: 29535530 |
| 63353 | 13: 29588319 | 63357 | 13: 29751352 | 63369 | 13: 30024809 | 63371 | 13: 30056042 | 63375 | 13: 30104514 |
| 63376 | 13: 30183328 | 63377 | 13: 30183348 | 63378 | 13: 30194908 | 63379 | 13: 30194914 | 63380 | 13: 30194921 |
| 63381 | 13: 30194933 | 63382 | 13: 30195026 | 63383 | 13: 30195496 | 63385 | 13: 30210577 | 63388 | 13: 30242235 |
| 63408 | 13: 30707478 | 63410 | 13: 30754086 | 63418 | 13: 30976341 | 63422 | 13: 31020731 | 63437 | 13: 31276749 |
| 63438 | 13: 31276801 | 63440 | 13: 31276837 | 63441 | 13: 31280242 | 63442 | 13: 31280491 | 63443 | 13: 31282636 |
| 63444 | 13: 31282742 | 63445 | 13: 31282787 | 63446 | 13: 31285967 | 63447 | 13: 31285968 | 63448 | 13: 31285972 |
| 63449 | 13: 31286001 | 63450 | 13: 31286035 | 63451 | 13: 31296423 | 63456 | 13: 31420376 | 63459 | 13: 31447543 |
| 63466 | 13: 31582342 | 63471 | 13: 31736295 | 63488 | 13: 32202239 | 63491 | 13: 32240332 | 63496 | 13: 32324178 |
| 63497 | 13: 32362115 | 63499 | 13: 32365033 | 63527 | 13: 32808897 | 63538 | 13: 33049577 | 63540 | 13: 33078146 |
| 63545 | 13: 33129981 | 63547 | 13: 33172919 | 63549 | 13: 33261503 | 63554 | 13: 33326303 | 63561 | 13: 33610056 |
| 63562 | 13: 33610145 | 63563 | 13: 33615532 | 63564 | 13: 33641897 | 63566 | 13: 33682852 | 63569 | 13: 33817581 |
| 63570 | 13: 33818040 | 63573 | 13: 33900260 | 63584 | 13: 34538815 | 63586 | 13: 34570424 | 63587 | 13: 34574425 |
| 63588 | 13: 34595352 | 63592 | 13: 34690679 | 63594 | 13: 34725177 | 63596 | 13: 34746582 | 63597 | 13: 34750823 |
| 63598 | 13: 34798730 | 63603 | 13: 34909612 | 63607 | 13: 35010608 | 63608 | 13: 35019310 | 63610 | 13: 35093002 |
| 63613 | 13: 35138794 | 63616 | 13: 35156141 | 63620 | 13: 35193780 | 63624 | 13: 35229937 | 63626 | 13: 35230259 |
| 63629 | 13: 35259809 | 63630 | 13: 35273048 | 63632 | 13: 35319711 | 63674 | 13: 36409030 | 63675 | 13: 36424590 |
| 63680 | 13: 36570662 | 63682 | 13: 36589718 | 63687 | 13: 36656277 | 63691 | 13: 36716029 | 63694 | 13: 36827015 |
| 63696 | 13: 36854156 | 63720 | 13: 37443281 | 63732 | 13: 37745409 | 63742 | 13: 37991091 | 63757 | 13: 38329758 |
| 63758 | 13: 38375463 | 63760 | 13: 38420421 | 63762 | 13: 38501340 | 63776 | 13: 38828149 | 63777 | 13: 38828176 |
| 63778 | 13: 38828210 | 63779 | 13: 38828306 | 63780 | 13: 38828333 | 63784 | 13: 38935644 | 63787 | 13: 38957716 |
| 63806 | 13: 39270018 | 63808 | 13: 39276440 | 63813 | 13: 39359409 | 63814 | 13: 39360537 | 63817 | 13: 39403535 |
| 63821 | 13: 39464756 | 63822 | 13: 39498069 | 63824 | 13: 39559729 | 63825 | 13: 39574123 | 63826 | 13: 39584391 |
| 63828 | 13: 39644803 | 63834 | 13: 39704818 | 63847 | 13: 40027121 | 63901 | 13: 41141472 | 63926 | 13: 41826984 |
| 63928 | 13: 41862833 | 63929 | 13: 41863502 | 63944 | 13: 42197339 | 63952 | 13: 42439893 | 63956 | 13: 42526261 |
| 63961 | 13: 42557806 | 63963 | 13: 42567447 | 63964 | 13: 42570926 | 63965 | 13: 42576337 | 63966 | 13: 42578756 |
| 63967 | 13: 42579747 | 63968 | 13: 42581051 | 63969 | 13: 42583033 | 63970 | 13: 42585954 | 63971 | 13: 42587350 |
| 63973 | 13: 42588548 | 63974 | 13: 42589734 | 63975 | 13: 42590562 | 63976 | 13: 42591034 | 63977 | 13: 42591591 |
| 63978 | 13: 42592688 | 63979 | 13: 42602259 | 63980 | 13: 42604226 | 63981 | 13: 42605060 | 63982 | 13: 42606861 |
| 63983 | 13: 42607539 | 63984 | 13: 42617225 | 63985 | 13: 42619772 | 63986 | 13: 42620639 | 63987 | 13: 42622791 |
| 63988 | 13: 42624718 | 63990 | 13: 42628160 | 63991 | 13: 42632412 | 63992 | 13: 42636254 | 63994 | 13: 42655005 |
| 63995 | 13: 42655747 | 63997 | 13: 42707046 | 63998 | 13: 42733395 | 64000 | 13: 42775404 | 64002 | 13: 42816216 |
| 64010 | 13: 43060399 | 64013 | 13: 43174367 | 64017 | 13: 43269543 | 64018 | 13: 43342598 | 64019 | 13: 43400200 |
| 64021 | 13: 43503495 | 64030 | 13: 43617243 | 64034 | 13: 43735619 | 64035 | 13: 43736152 | 64061 | 13: 44136436 |
| 64064 | 13: 44159827 | 64066 | 13: 44231058 | 64070 | 13: 44319398 | 64073 | 13: 44364216 | 64079 | 13: 44475918 |
| 64080 | 13: 44491717 | 64091 | 13: 44736057 | 64116 | 13: 44978580 | 64117 | 13: 44978611 | 64119 | 13: 44989622 |
| 64128 | 13: 45018113 | 64129 | 13: 45022424 | 64130 | 13: 45034606 | 64132 | 13: 45038863 | 64133 | 13: 45046560 |
| 64135 | 13: 45057233 | 64136 | 13: 45081132 | 64138 | 13: 45099582 | 64139 | 13: 45108541 | 64140 | 13: 45110718 |
| 64141 | 13: 45112674 | 64142 | 13: 45114484 | 64144 | 13: 45129342 | 64145 | 13: 45135756 | 64146 | 13: 45143400 |
| 64147 | 13: 45144409 | 64149 | 13: 45160765 | 64173 | 13: 45837065 | 64188 | 13: 46248201 | 64189 | 13: 46260736 |
| 64190 | 13: 46262101 | 64191 | 13: 46280773 | 64192 | 13: 46303988 | 64193 | 13: 46308862 | 64197 | 13: 46342659 |
| 64203 | 13: 46433525 | 64205 | 13: 46541983 | 64217 | 13: 46759585 | 64219 | 13: 46767424 | 64222 | 13: 46855501 |
| 64226 | 13: 46934942 | 64230 | 13: 47057469 | 64235 | 13: 47187424 | 64240 | 13: 47305456 | 64242 | 13: 47333121 |
| 64243 | 13: 47346135 | 64265 | 13: 47860032 | 64267 | 13: 47916526 | 64268 | 13: 47927079 | 64270 | 13: 47952062 |
| 64271 | 13: 48003258 | 64272 | 13: 48017728 | 64279 | 13: 48210178 | 64281 | 13: 48212858 | 64313 | 13: 48933264 |
| 64327 | 13: 49369652 | 64371 | 13: 50683553 | 64377 | 13: 50955038 | 64379 | 13: 50985036 | 64385 | 13: 51191490 |
| 64388 | 13: 51268713 | 64389 | 13: 51269069 | 64404 | 13: 51645253 | 64408 | 13: 51745594 | 64412 | 13: 51889992 |
| 64413 | 13: 51917960 | 64424 | 13: 52280855 | 64425 | 13: 52333810 | 64430 | 13: 52514427 | 64436 | 13: 52666460 |
| 64443 | 13: 52794690 | 64444 | 13: 52797973 | 64445 | 13: 52798735 | 64446 | 13: 52798786 | 64448 | 13: 52806170 |
| 64450 | 13: 52838262 | 64457 | 13: 53001236 | 64461 | 13: 53139865 | 64463 | 13: 53146985 | 64465 | 13: 53149275 |
| 64468 | 13: 53184275 | 64470 | 13: 53189308 | 64471 | 13: 53191327 | 64472 | 13: 53192744 | 64473 | 13: 53193885 |
| 64474 | 13: 53195323 | 64475 | 13: 53197934 | 64476 | 13: 53199665 | 64477 | 13: 53200513 | 64478 | 13: 53201494 |
| 64479 | 13: 53205894 | 64480 | 13: 53211390 | 64481 | 13: 53212642 | 64482 | 13: 53213246 | 64483 | 13: 53214256 |
| 64484 | 13: 53221957 | 64485 | 13: 53223771 | 64486 | 13: 53225335 | 64490 | 13: 53267383 | 64491 | 13: 53287399 |
| 64502 | 13: 53656779 | 64503 | 13: 53668817 | 64521 | 13: 54501975 | 64524 | 13: 54544791 | 64528 | 13: 54637805 |
| 64532 | 13: 54688808 | 64539 | 13: 54867617 | 64540 | 13: 54924099 | 64541 | 13: 54930460 | 64542 | 13: 54963877 |
| 64545 | 13: 55001775 | 64546 | 13: 55009029 | 64549 | 13: 55107919 | 64551 | 13: 55137013 | 64553 | 13: 55164494 |
| 64556 | 13: 55256797 | 64558 | 13: 55286529 | 64559 | 13: 55306487 | 64563 | 13: 55363256 | 64576 | 13: 55652395 |
| 64579 | 13: 55736298 | 64586 | 13: 55924074 | 64596 | 13: 56129806 | 64599 | 13: 56206409 | 64602 | 13: 56257733 |
| 64613 | 13: 56553088 | 64615 | 13: 56567143 | 64616 | 13: 56574066 | 64628 | 13: 56987530 | 64632 | 13: 57050196 |
| 64642 | 13: 57391199 | 64643 | 13: 57394025 | 64654 | 13: 57698221 | 64655 | 13: 57736295 | 64656 | 13: 57744564 |
| 64657 | 13: 57744624 | 64664 | 13: 57962862 | 64677 | 13: 58287555 | 64678 | 13: 58288391 | 64679 | 13: 58297881 |
| 64683 | 13: 58337666 | 64688 | 13: 58408741 | 64689 | 13: 58453137 | 64690 | 13: 58475365 | 64693 | 13: 58521548 |
| 64700 | 13: 58687733 | 64703 | 13: 58755844 | 64705 | 13: 58823450 | 64709 | 13: 58925642 | 64711 | 13: 58935983 |
| 64713 | 13: 58968219 | 64718 | 13: 58981092 | 64737 | 13: 59317532 | 64738 | 13: 59317573 | 64739 | 13: 59317600 |
| 64752 | 13: 59798919 | 64753 | 13: 59810522 | 64757 | 13: 59881900 | 64763 | 13: 59982505 | 64765 | 13: 60004397 |
| 64775 | 13: 60191346 | 64777 | 13: 60219559 | 64779 | 13: 60249957 | 64781 | 13: 60270738 | 64782 | 13: 60273459 |
| 64786 | 13: 60360621 | 64787 | 13: 60363893 | 64803 | 13: 60828808 | 64813 | 13: 61235720 | 64819 | 13: 61487750 |
| 64823 | 13: 61526661 | 64824 | 13: 61536675 | 64830 | 13: 61748180 | 64833 | 13: 61837125 | 64857 | 13: 62340129 |
| 64864 | 13: 62437505 | 64865 | 13: 62437664 | 64869 | 13: 62616409 | 64870 | 13: 62619495 | 64871 | 13: 62620895 |
| 64872 | 13: 62620917 | 64873 | 13: 62623230 | 64874 | 13: 62623246 | 64875 | 13: 62623260 | 64876 | 13: 62623271 |
| 64877 | 13: 62623277 | 64878 | 13: 62623296 | 64879 | 13: 62623326 | 64890 | 13: 62758151 | 64895 | 13: 62805225 |
| 64896 | 13: 62808252 | 64898 | 13: 62825728 | 64903 | 13: 62901932 | 64904 | 13: 62913802 | 64905 | 13: 62957243 |
| 64909 | 13: 63100717 | 64910 | 13: 63113668 | 64911 | 13: 63115815 | 64912 | 13: 63117671 | 64915 | 13: 63231676 |
| 64916 | 13: 63238472 | 64922 | 13: 63381167 | 64924 | 13: 63406383 | 64927 | 13: 63465271 | 64932 | 13: 63632674 |
| 64933 | 13: 63667387 | 64936 | 13: 63800029 | 64941 | 13: 63926705 | 64944 | 13: 63945489 | 64945 | 13: 63950962 |
| 64964 | 13: 64427414 | 64970 | 13: 64579641 | 64973 | 13: 64608490 | 64979 | 13: 64765574 | 64981 | 13: 64780081 |
| 64985 | 13: 64917447 | 64992 | 13: 65197131 | 64993 | 13: 65222405 | 64994 | 13: 65226257 | 64996 | 13: 65300967 |
| 65002 | 13: 65421703 | 65003 | 13: 65421742 | 65008 | 13: 65551485 | 65009 | 13: 65556446 | 65012 | 13: 65615123 |
| 65013 | 13: 65633719 | 65015 | 13: 65678606 | 65016 | 13: 65710639 | 65017 | 13: 65711755 | 65018 | 13: 65713224 |
| 65019 | 13: 65713273 | 65020 | 13: 65714024 | 65022 | 13: 65714678 | 65024 | 13: 65734511 | 65025 | 13: 65737670 |
| 65026 | 13: 65747364 | 65027 | 13: 65751601 | 65028 | 13: 65757675 | 65029 | 13: 65768152 | 65030 | 13: 65769345 |
| 65031 | 13: 65771689 | 65032 | 13: 65778554 | 65038 | 13: 65891788 | 65045 | 13: 66156547 | 65051 | 13: 66242964 |
| 65054 | 13: 66269753 | 65058 | 13: 66340843 | 65068 | 13: 66611398 | 65078 | 13: 66830214 | 65084 | 13: 66995569 |
| 65087 | 13: 67120106 | 65103 | 13: 67448820 | 65126 | 13: 68187572 | 65134 | 13: 68308181 | 65136 | 13: 68378513 |
| 65138 | 13: 68519206 | 65140 | 13: 68528941 | 65142 | 13: 68635086 | 65146 | 13: 68672438 | 65152 | 13: 68880703 |
| 65165 | 13: 69217542 | 65168 | 13: 69272129 | 65170 | 13: 69348741 | 65171 | 13: 69353425 | 65173 | 13: 69380590 |
| 65183 | 13: 69728600 | 65184 | 13: 69731770 | 65187 | 13: 69850661 | 65189 | 13: 69882420 | 65190 | 13: 69890851 |
| 65198 | 13: 70040165 | 65201 | 13: 70082692 | 65207 | 13: 70199117 | 65220 | 13: 70568959 | 65225 | 13: 70651503 |
| 65227 | 13: 70673685 | 65248 | 13: 71190284 | 65267 | 13: 71537155 | 65272 | 13: 71649810 | 65276 | 13: 71778746 |
| 65278 | 13: 71794746 | 65289 | 13: 72099141 | 65290 | 13: 72099160 | 65293 | 13: 72190824 | 65298 | 13: 72223856 |
| 65299 | 13: 72240445 | 65300 | 13: 72247438 | 65308 | 13: 72487250 | 65311 | 13: 72581815 | 65316 | 13: 72835577 |
| 65318 | 13: 72837581 | 65319 | 13: 72837712 | 65320 | 13: 72837736 | 65323 | 13: 72873627 | 65326 | 13: 72911160 |
| 65327 | 13: 72931732 | 65331 | 13: 72989401 | 65333 | 13: 73030191 | 65342 | 13: 73241000 | 65353 | 13: 73412480 |
| 65358 | 13: 73475168 | 65359 | 13: 73486255 | 65360 | 13: 73508637 | 65361 | 13: 73516499 | 65362 | 13: 73552702 |
| 65363 | 13: 73554377 | 65367 | 13: 73602251 | 65369 | 13: 73628006 | 65370 | 13: 73628032 | 65372 | 13: 73657151 |
| 65373 | 13: 73659243 | 65375 | 13: 73684336 | 65376 | 13: 73720189 | 65377 | 13: 73738431 | 65378 | 13: 73747745 |
| 65386 | 13: 74002990 | 65388 | 13: 74075881 | 65396 | 13: 74272611 | 65397 | 13: 74284172 | 65405 | 13: 74425124 |
| 65406 | 13: 74461134 | 65407 | 13: 74464989 | 65408 | 13: 74467020 | 65409 | 13: 74470296 | 65410 | 13: 74476473 |
| 65411 | 13: 74479132 | 65412 | 13: 74479161 | 65417 | 13: 74575589 | 65419 | 13: 74593675 | 65421 | 13: 74635628 |
| 65423 | 13: 74640625 | 65426 | 13: 74665130 | 65427 | 13: 74678375 | 65432 | 13: 74760409 | 65433 | 13: 74815833 |
| 65437 | 13: 74932791 | 65455 | 13: 75521831 | 65463 | 13: 75584984 | 65464 | 13: 75584987 | 65465 | 13: 75585044 |
| 65473 | 13: 75757004 | 65474 | 13: 75767512 | 65478 | 13: 75797537 | 65479 | 13: 75808921 | 65481 | 13: 75817367 |
| 65496 | 13: 76209741 | 65503 | 13: 76376258 | 65506 | 13: 76438473 | 65507 | 13: 76438492 | 65508 | 13: 76442151 |
| 65509 | 13: 76443128 | 65521 | 13: 76672357 | 65523 | 13: 76717525 | 65527 | 13: 76772665 | 65534 | 13: 76884649 |
| 65545 | 13: 77225860 | 65551 | 13: 77281427 | 65555 | 13: 77332710 | 65557 | 13: 77387866 | 65560 | 13: 77423447 |
| 65561 | 13: 77443501 | 65572 | 13: 77715514 | 65574 | 13: 77719500 | 65576 | 13: 77810786 | 65579 | 13: 77851241 |
| 65580 | 13: 77856986 | 65584 | 13: 78019495 | 65589 | 13: 78097466 | 65590 | 13: 78097543 | 65591 | 13: 78097572 |
| 65595 | 13: 78246967 | 65600 | 13: 78323978 | 65601 | 13: 78327563 | 65611 | 13: 78606738 | 65620 | 13: 78732308 |
| 65628 | 13: 78862443 | 65630 | 13: 78895292 | 65636 | 13: 79010034 | 65646 | 13: 79138574 | 65653 | 13: 79289518 |
| 65663 | 13: 79372632 | 65664 | 13: 79378945 | 65670 | 13: 79439113 | 65746 | 13: 80685975 | 65753 | 13: 80868488 |
| 65754 | 13: 80869352 | 65775 | 13: 81361992 | 65780 | 13: 81441602 | 65789 | 13: 81578319 | 65793 | 13: 81659411 |
| 65796 | 13: 81695713 | 65809 | 13: 81975437 | 65811 | 13: 82042702 | 65857 | 13: 82944108 | 65859 | 13: 82944150 |
| 65870 | 13: 83194337 | 65885 | 14: 260388 | 65890 | 14: 344042 | 65892 | 14: 428398 | 65906 | 14: 1024267 |
| 65907 | 14: 1025055 | 65908 | 14: 1033297 | 65909 | 14: 1036134 | 65910 | 14: 1039518 | 65911 | 14: 1044777 |
| 65914 | 14: 1153408 | 65916 | 14: 1188418 | 65919 | 14: 1314992 | 65923 | 14: 1388615 | 65924 | 14: 1394179 |
| 65928 | 14: 1554543 | 65930 | 14: 1658546 | 65931 | 14: 1670892 | 65932 | 14: 1671183 | 65933 | 14: 1671573 |
| 65934 | 14: 1671580 | 65935 | 14: 1681783 | 65936 | 14: 1702340 | 65940 | 14: 1785051 | 65941 | 14: 1798788 |
| 65942 | 14: 1799618 | 65943 | 14: 1799673 | 65944 | 14: 1800049 | 65946 | 14: 1844464 | 65947 | 14: 1854457 |
| 65950 | 14: 1926589 | 65951 | 14: 1946346 | 65966 | 14: 2415920 | 65968 | 14: 2500509 | 65973 | 14: 2669589 |
| 65982 | 14: 2854696 | 65988 | 14: 3015800 | 65989 | 14: 3040833 | 65990 | 14: 3040885 | 65994 | 14: 3097950 |
| 65998 | 14: 3147254 | 66005 | 14: 3206294 | 66006 | 14: 3212884 | 66007 | 14: 3218403 | 66008 | 14: 3218478 |
| 66009 | 14: 3218557 | 66010 | 14: 3221269 | 66011 | 14: 3221338 | 66012 | 14: 3239650 | 66013 | 14: 3239696 |
| 66014 | 14: 3239752 | 66015 | 14: 3239754 | 66016 | 14: 3242336 | 66017 | 14: 3246999 | 66018 | 14: 3247042 |
| 66019 | 14: 3247052 | 66020 | 14: 3278835 | 66021 | 14: 3278838 | 66022 | 14: 3284409 | 66023 | 14: 3284439 |
| 66024 | 14: 3298378 | 66025 | 14: 3305527 | 66026 | 14: 3308936 | 66028 | 14: 3336128 | 66029 | 14: 3336263 |
| 66034 | 14: 3386889 | 66036 | 14: 3433819 | 66038 | 14: 3453301 | 66041 | 14: 3471847 | 66080 | 14: 4216507 |
| 66082 | 14: 4228026 | 66083 | 14: 4228352 | 66085 | 14: 4232919 | 66086 | 14: 4233543 | 66113 | 14: 4912069 |
| 66118 | 14: 5004897 | 66178 | 14: 5998302 | 66185 | 14: 6071169 | 66191 | 14: 6163665 | 66192 | 14: 6174543 |
| 66203 | 14: 6285204 | 66206 | 14: 6295145 | 66207 | 14: 6303201 | 66212 | 14: 6388522 | 66214 | 14: 6472930 |
| 66237 | 14: 6859614 | 66248 | 14: 7064392 | 66250 | 14: 7109542 | 66252 | 14: 7127144 | 66269 | 14: 7497044 |
| 66274 | 14: 7545925 | 66275 | 14: 7545927 | 66276 | 14: 7545936 | 66280 | 14: 7626758 | 66294 | 14: 7895151 |
| 66300 | 14: 7989733 | 66311 | 14: 8058621 | 66312 | 14: 8058649 | 66316 | 14: 8179389 | 66321 | 14: 8301355 |
| 66323 | 14: 8312764 | 66327 | 14: 8394739 | 66328 | 14: 8416607 | 66329 | 14: 8424727 | 66331 | 14: 8441111 |
| 66333 | 14: 8445298 | 66337 | 14: 8540399 | 66338 | 14: 8551277 | 66340 | 14: 8561525 | 66341 | 14: 8566274 |
| 66343 | 14: 8579200 | 66346 | 14: 8601680 | 66347 | 14: 8601681 | 66349 | 14: 8629638 | 66353 | 14: 8664840 |
| 66354 | 14: 8664847 | 66356 | 14: 8672236 | 66359 | 14: 8707161 | 66363 | 14: 8798924 | 66375 | 14: 9035591 |
| 66378 | 14: 9063069 | 66379 | 14: 9064094 | 66380 | 14: 9064111 | 66381 | 14: 9064154 | 66382 | 14: 9064258 |
| 66384 | 14: 9095754 | 66386 | 14: 9121381 | 66387 | 14: 9140334 | 66388 | 14: 9140361 | 66389 | 14: 9140418 |
| 66390 | 14: 9141498 | 66394 | 14: 9188175 | 66400 | 14: 9361706 | 66402 | 14: 9382539 | 66403 | 14: 9399137 |
| 66404 | 14: 9401991 | 66430 | 14: 9976682 | 66431 | 14: 9976704 | 66432 | 14: 9976709 | 66434 | 14: 10038814 |
| 66438 | 14: 10102756 | 66440 | 14: 10152585 | 66443 | 14: 10280749 | 66451 | 14: 10486501 | 66455 | 14: 10530506 |
| 66457 | 14: 10557006 | 66458 | 14: 10566461 | 66459 | 14: 10577632 | 66462 | 14: 10706026 | 66464 | 14: 10756790 |
| 66465 | 14: 10764173 | 66466 | 14: 10788680 | 66467 | 14: 10801527 | 66468 | 14: 10805790 | 66469 | 14: 10807806 |
| 66470 | 14: 10816880 | 66474 | 14: 10893686 | 66476 | 14: 10914761 | 66479 | 14: 10947558 | 66481 | 14: 11009369 |
| 66484 | 14: 11088275 | 66492 | 14: 11252807 | 66504 | 14: 11411289 | 66505 | 14: 11444087 | 66510 | 14: 11540331 |
| 66512 | 14: 11568465 | 66513 | 14: 11575517 | 66516 | 14: 11596900 | 66517 | 14: 11657154 | 66526 | 14: 11763629 |
| 66533 | 14: 11962212 | 66541 | 14: 12136166 | 66546 | 14: 12207418 | 66551 | 14: 12292359 | 66552 | 14: 12293976 |
| 66554 | 14: 12315574 | 66558 | 14: 12384353 | 66559 | 14: 12388961 | 66560 | 14: 12393160 | 66564 | 14: 12514540 |
| 66578 | 14: 12695561 | 66579 | 14: 12710347 | 66593 | 14: 13094541 | 66595 | 14: 13178679 | 66618 | 14: 13920366 |
| 66623 | 14: 14023534 | 66628 | 14: 14069858 | 66631 | 14: 14135529 | 66636 | 14: 14326793 | 66646 | 14: 14560727 |
| 66649 | 14: 14707903 | 66651 | 14: 14728697 | 66652 | 14: 14744411 | 66656 | 14: 14812773 | 66657 | 14: 14823932 |
| 66662 | 14: 14952020 | 66664 | 14: 15013585 | 66666 | 14: 15072256 | 66668 | 14: 15100414 | 66675 | 14: 15318937 |
| 66677 | 14: 15351351 | 66678 | 14: 15369492 | 66682 | 14: 15454870 | 66689 | 14: 15529862 | 66704 | 14: 15827253 |
| 66710 | 14: 15926072 | 66712 | 14: 15954837 | 66715 | 14: 15972218 | 66723 | 14: 16143522 | 66725 | 14: 16177691 |
| 66730 | 14: 16258523 | 66734 | 14: 16293114 | 66737 | 14: 16436394 | 66738 | 14: 16442429 | 66739 | 14: 16450949 |
| 66741 | 14: 16479769 | 66742 | 14: 16493894 | 66752 | 14: 16722564 | 66753 | 14: 16790460 | 66754 | 14: 16817518 |
| 66756 | 14: 16852507 | 66761 | 14: 16906633 | 66762 | 14: 16939530 | 66763 | 14: 16939542 | 66764 | 14: 16939598 |
| 66765 | 14: 16939628 | 66767 | 14: 16969260 | 66771 | 14: 17041215 | 66778 | 14: 17280279 | 66798 | 14: 17770696 |
| 66799 | 14: 17770830 | 66801 | 14: 17799708 | 66802 | 14: 17816110 | 66803 | 14: 17843985 | 66836 | 14: 18601570 |
| 66849 | 14: 18880871 | 66860 | 14: 19181912 | 66865 | 14: 19283893 | 66869 | 14: 19432077 | 66881 | 14: 19699930 |
| 66882 | 14: 19743977 | 66885 | 14: 19813853 | 66890 | 14: 19975802 | 66893 | 14: 19991875 | 66927 | 14: 20847256 |
| 66930 | 14: 20893054 | 66931 | 14: 20896371 | 66932 | 14: 20913913 | 66935 | 14: 20978169 | 66937 | 14: 21048592 |
| 66958 | 14: 21485350 | 66963 | 14: 21604476 | 66974 | 14: 21869418 | 66977 | 14: 21931720 | 66979 | 14: 21998392 |
| 66984 | 14: 22161961 | 66995 | 14: 22407071 | 66996 | 14: 22407076 | 67077 | 14: 22732607 | 67093 | 14: 22882106 |
| 67095 | 14: 22886422 | 67173 | 14: 23215974 | 67345 | 14: 23539160 | 67346 | 14: 23539175 | 67347 | 14: 23539193 |
| 67348 | 14: 23539196 | 67349 | 14: 23539229 | 67424 | 14: 23981200 | 67428 | 14: 24041313 | 67443 | 14: 24389626 |
| 67445 | 14: 24408833 | 67514 | 14: 25111427 | 67519 | 14: 25203874 | 67520 | 14: 25205074 | 67525 | 14: 25258907 |
| 67529 | 14: 25432157 | 67561 | 14: 25864255 | 67565 | 14: 25923336 | 67566 | 14: 25923373 | 67568 | 14: 25985806 |
| 67570 | 14: 25990906 | 67571 | 14: 26012359 | 67573 | 14: 26052398 | 67575 | 14: 26065694 | 67580 | 14: 26140450 |
| 67581 | 14: 26140990 | 67582 | 14: 26141962 | 67583 | 14: 26143779 | 67585 | 14: 26163203 | 67586 | 14: 26170631 |
| 67587 | 14: 26175012 | 67588 | 14: 26176715 | 67590 | 14: 26182412 | 67591 | 14: 26182943 | 67593 | 14: 26184111 |
| 67594 | 14: 26184775 | 67595 | 14: 26185527 | 67596 | 14: 26186351 | 67597 | 14: 26190407 | 67598 | 14: 26191236 |
| 67600 | 14: 26193748 | 67601 | 14: 26194782 | 67602 | 14: 26195880 | 67603 | 14: 26197356 | 67604 | 14: 26209375 |
| 67605 | 14: 26210727 | 67606 | 14: 26212648 | 67607 | 14: 26216203 | 67608 | 14: 26225153 | 67610 | 14: 26246778 |
| 67611 | 14: 26271742 | 67614 | 14: 26308735 | 67617 | 14: 26333506 | 67620 | 14: 26432685 | 67626 | 14: 26633301 |
| 67632 | 14: 26717685 | 67648 | 14: 27181625 | 67649 | 14: 27210120 | 67650 | 14: 27233742 | 67670 | 14: 27952519 |
| 67673 | 14: 28069983 | 67685 | 14: 28479844 | 67691 | 14: 28620485 | 67692 | 14: 28622173 | 67693 | 14: 28624764 |
| 67697 | 14: 28758622 | 67715 | 14: 29296020 | 67725 | 14: 29459640 | 67740 | 14: 29698665 | 67748 | 14: 29894372 |
| 67749 | 14: 29989315 | 67752 | 14: 30012586 | 67767 | 14: 30342088 | 67768 | 14: 30349359 | 67769 | 14: 30356890 |
| 67778 | 14: 30465481 | 67781 | 14: 30499744 | 67794 | 14: 30690571 | 67797 | 14: 30764962 | 67798 | 14: 30811197 |
| 67808 | 14: 31180467 | 67819 | 14: 31555900 | 67820 | 14: 31555992 | 67826 | 14: 31641997 | 67833 | 14: 31874739 |
| 67836 | 14: 31944000 | 67844 | 14: 32203109 | 67866 | 14: 32398203 | 67886 | 14: 32981178 | 67890 | 14: 33060155 |
| 67896 | 14: 33143197 | 67897 | 14: 33143201 | 67900 | 14: 33275732 | 67906 | 14: 33326342 | 67907 | 14: 33342080 |
| 67912 | 14: 33449262 | 67913 | 14: 33458276 | 67919 | 14: 33529983 | 67930 | 14: 33731278 | 67932 | 14: 33839737 |
| 67938 | 14: 33897507 | 67939 | 14: 33921705 | 67942 | 14: 33981693 | 67947 | 14: 34164312 | 67953 | 14: 34301611 |
| 67959 | 14: 34528656 | 67975 | 14: 35058048 | 68018 | 14: 36043204 | 68028 | 14: 36299508 | 68035 | 14: 36373476 |
| 68052 | 14: 36852980 | 68071 | 14: 37488132 | 68084 | 14: 37849675 | 68085 | 14: 37852302 | 68089 | 14: 37925483 |
| 68097 | 14: 38188466 | 68115 | 14: 38814239 | 68116 | 14: 38815880 | 68117 | 14: 38822128 | 68118 | 14: 38822260 |
| 68141 | 14: 39497949 | 68155 | 14: 39876130 | 68156 | 14: 39966401 | 68160 | 14: 40176632 | 68161 | 14: 40176648 |
| 68162 | 14: 40176909 | 68163 | 14: 40176917 | 68164 | 14: 40176996 | 68165 | 14: 40177014 | 68166 | 14: 40177030 |
| 68167 | 14: 40177236 | 68168 | 14: 40177299 | 68179 | 14: 40478861 | 68203 | 14: 41361918 | 68205 | 14: 41438833 |
| 68208 | 14: 41573733 | 68214 | 14: 41689230 | 68217 | 14: 41730144 | 68218 | 14: 41737864 | 68242 | 14: 42286395 |
| 68254 | 14: 42523669 | 68264 | 14: 42805270 | 68273 | 14: 43158811 | 68280 | 14: 43440082 | 68288 | 14: 43769450 |
| 68307 | 14: 44309555 | 68309 | 14: 44430721 | 68318 | 14: 44636417 | 68322 | 14: 44676672 | 68323 | 14: 44676728 |
| 68324 | 14: 44677183 | 68326 | 14: 44690793 | 68327 | 14: 44724666 | 68329 | 14: 44762792 | 68331 | 14: 44776625 |
| 68334 | 14: 44843603 | 68335 | 14: 44854809 | 68336 | 14: 44856580 | 68340 | 14: 44966227 | 68344 | 14: 44988177 |
| 68345 | 14: 45015405 | 68346 | 14: 45018776 | 68347 | 14: 45022201 | 68348 | 14: 45041057 | 68356 | 14: 45253129 |
| 68357 | 14: 45264773 | 68362 | 14: 45445260 | 68378 | 14: 45823929 | 68379 | 14: 45842554 | 68380 | 14: 45845229 |
| 68387 | 14: 45960737 | 68392 | 14: 46043332 | 68395 | 14: 46074189 | 68416 | 14: 46394434 | 68419 | 14: 46425925 |
| 68425 | 14: 46627739 | 68427 | 14: 46701293 | 68437 | 14: 46893352 | 68447 | 14: 47088081 | 68477 | 14: 48079880 |
| 68486 | 14: 48324535 | 68488 | 14: 48324675 | 68491 | 14: 48337571 | 68505 | 14: 48683313 | 68508 | 14: 48746367 |
| 68510 | 14: 48777365 | 68531 | 14: 49324283 | 68534 | 14: 49446616 | 68535 | 14: 49446636 | 68557 | 14: 50162370 |
| 68562 | 14: 50269979 | 68571 | 14: 50641123 | 68586 | 14: 51157346 | 68593 | 14: 51380992 | 68594 | 14: 51381093 |
| 68599 | 14: 51513649 | 68609 | 14: 51705820 | 68618 | 14: 51969375 | 68621 | 14: 52011701 | 68622 | 14: 52087941 |
| 68624 | 14: 52117812 | 68626 | 14: 52174077 | 68627 | 14: 52174084 | 68628 | 14: 52176416 | 68629 | 14: 52176503 |
| 68630 | 14: 52181003 | 68631 | 14: 52193756 | 68632 | 14: 52206425 | 68634 | 14: 52240902 | 68635 | 14: 52240959 |
| 68636 | 14: 52272882 | 68637 | 14: 52274310 | 68647 | 14: 52598698 | 68666 | 14: 53395458 | 68667 | 14: 53433645 |
| 68678 | 14: 53716855 | 68694 | 14: 54155951 | 68699 | 14: 54289749 | 68703 | 14: 54322218 | 68704 | 14: 54323172 |
| 68705 | 14: 54324711 | 68706 | 14: 54326865 | 68707 | 14: 54328568 | 68708 | 14: 54329372 | 68709 | 14: 54330950 |
| 68710 | 14: 54331798 | 68711 | 14: 54332484 | 68712 | 14: 54333187 | 68713 | 14: 54334454 | 68714 | 14: 54335896 |
| 68715 | 14: 54340206 | 68718 | 14: 54366857 | 68719 | 14: 54375801 | 68722 | 14: 54397201 | 68723 | 14: 54441985 |
| 68725 | 14: 54458565 | 68732 | 14: 54625281 | 68745 | 14: 54834484 | 68749 | 14: 55039582 | 68751 | 14: 55074676 |
| 68756 | 14: 55222131 | 68757 | 14: 55226008 | 68758 | 14: 55230280 | 68759 | 14: 55230389 | 68760 | 14: 55283357 |
| 68769 | 14: 55484731 | 68780 | 14: 55685950 | 68789 | 14: 56225330 | 68790 | 14: 56256482 | 68791 | 14: 56276255 |
| 68799 | 14: 56506951 | 68803 | 14: 56545284 | 68804 | 14: 56555253 | 68808 | 14: 56652939 | 68816 | 14: 56827541 |
| 68831 | 14: 57332067 | 68832 | 14: 57346169 | 68833 | 14: 57350713 | 68834 | 14: 57366372 | 68835 | 14: 57383485 |
| 68839 | 14: 57448890 | 68840 | 14: 57459060 | 68842 | 14: 57536798 | 68846 | 14: 57585046 | 68851 | 14: 57728888 |
| 68852 | 14: 57728921 | 68853 | 14: 57779780 | 68854 | 14: 57796975 | 68856 | 14: 57816918 | 68859 | 14: 57922597 |
| 68860 | 14: 57935303 | 68864 | 14: 58056243 | 68868 | 14: 58089515 | 68874 | 14: 58220993 | 68885 | 14: 58495696 |
| 68886 | 14: 58510271 | 68888 | 14: 58533542 | 68892 | 14: 58668510 | 68905 | 14: 58947933 | 68910 | 14: 59026237 |
| 68912 | 14: 59043151 | 68920 | 14: 59231026 | 68947 | 14: 59935092 | 68953 | 14: 60142373 | 68957 | 14: 60181604 |
| 68958 | 14: 60181624 | 68964 | 14: 60282711 | 68965 | 14: 60298957 | 68967 | 14: 60335923 | 68969 | 14: 60400617 |
| 68973 | 14: 60573159 | 68977 | 14: 60646587 | 68978 | 14: 60646600 | 68983 | 14: 60827415 | 68990 | 14: 60986477 |
| 68994 | 14: 61146297 | 68996 | 14: 61230816 | 68997 | 14: 61230819 | 69001 | 14: 61287942 | 69002 | 14: 61313591 |
| 69003 | 14: 61315627 | 69007 | 14: 61411151 | 69008 | 14: 61412641 | 69009 | 14: 61412766 | 69010 | 14: 61412805 |
| 69011 | 14: 61414085 | 69014 | 14: 61513794 | 69028 | 14: 61777801 | 69033 | 14: 61849353 | 69040 | 14: 61948505 |
| 69044 | 14: 62039648 | 69046 | 14: 62081343 | 69049 | 14: 62134019 | 69052 | 14: 62144762 | 69053 | 14: 62144767 |
| 69054 | 14: 62149426 | 69060 | 14: 62299231 | 69068 | 14: 62442888 | 69073 | 14: 62565653 | 69078 | 14: 62687349 |
| 69088 | 14: 62815477 | 69090 | 14: 62881003 | 69100 | 14: 63151822 | 69103 | 14: 63198642 | 69104 | 14: 63225917 |
| 69113 | 14: 63395803 | 69128 | 14: 63850266 | 69131 | 14: 63928813 | 69146 | 14: 64220537 | 69149 | 14: 64336877 |
| 69150 | 14: 64339120 | 69151 | 14: 64353146 | 69156 | 14: 64514888 | 69194 | 14: 65705048 | 69201 | 14: 65784254 |
| 69206 | 14: 65890326 | 69211 | 14: 66085117 | 69212 | 14: 66121609 | 69213 | 14: 66141319 | 69221 | 14: 66266296 |
| 69222 | 14: 66266410 | 69224 | 14: 66350819 | 69225 | 14: 66350850 | 69233 | 14: 66424408 | 69237 | 14: 66604661 |
| 69240 | 14: 66662602 | 69241 | 14: 66669902 | 69242 | 14: 66669945 | 69247 | 14: 66766263 | 69263 | 14: 67192810 |
| 69265 | 14: 67258930 | 69268 | 14: 67326499 | 69270 | 14: 67366402 | 69274 | 14: 67469984 | 69275 | 14: 67472732 |
| 69276 | 14: 67473297 | 69277 | 14: 67473813 | 69278 | 14: 67478743 | 69279 | 14: 67483782 | 69280 | 14: 67495613 |
| 69284 | 14: 67550636 | 69287 | 14: 67603655 | 69290 | 14: 67630731 | 69292 | 14: 67655532 | 69293 | 14: 67660678 |
| 69299 | 14: 67907866 | 69318 | 14: 68256914 | 69323 | 14: 68358639 | 69355 | 14: 69386387 | 69366 | 14: 69774142 |
| 69367 | 14: 69787949 | 69368 | 14: 69802911 | 69371 | 14: 69913935 | 69373 | 14: 69940920 | 69375 | 14: 70002759 |
| 69381 | 14: 70075613 | 69385 | 14: 70170918 | 69386 | 14: 70171506 | 69388 | 14: 70172986 | 69392 | 14: 70218397 |
| 69398 | 14: 70356280 | 69401 | 14: 70440463 | 69403 | 14: 70472924 | 69412 | 14: 70696424 | 69418 | 14: 70903085 |
| 69420 | 14: 70964025 | 69440 | 14: 71594264 | 69447 | 14: 71762783 | 69459 | 14: 72082157 | 69485 | 14: 72739791 |
| 69489 | 14: 72764165 | 69499 | 14: 73108865 | 69504 | 14: 73206090 | 69507 | 14: 73232071 | 69510 | 14: 73246685 |
| 69516 | 14: 73444151 | 69525 | 14: 73731378 | 69529 | 14: 73806578 | 69578 | 14: 75097229 | 69579 | 14: 75097255 |
| 69582 | 14: 75218040 | 69586 | 14: 75379695 | 69587 | 14: 75379698 | 69588 | 14: 75379711 | 69592 | 14: 75591751 |
| 69596 | 14: 75706688 | 69611 | 14: 76100504 | 69612 | 14: 76100657 | 69615 | 14: 76128821 | 69616 | 14: 76129624 |
| 69617 | 14: 76132580 | 69618 | 14: 76138175 | 69619 | 14: 76141064 | 69621 | 14: 76142867 | 69622 | 14: 76144495 |
| 69625 | 14: 76178839 | 69626 | 14: 76178890 | 69628 | 14: 76195852 | 69629 | 14: 76199476 | 69630 | 14: 76207317 |
| 69635 | 14: 76328357 | 69637 | 14: 76366242 | 69646 | 14: 76503558 | 69653 | 14: 76612609 | 69655 | 14: 76770661 |
| 69656 | 14: 76771798 | 69659 | 14: 76845625 | 69661 | 14: 76923277 | 69663 | 14: 76945033 | 69664 | 14: 76964292 |
| 69670 | 14: 77099484 | 69671 | 14: 77174586 | 69672 | 14: 77206279 | 69681 | 14: 77471278 | 69682 | 14: 77471456 |
| 69684 | 14: 77512276 | 69712 | 14: 78249703 | 69713 | 14: 78249764 | 69714 | 14: 78249780 | 69720 | 14: 78305043 |
| 69721 | 14: 78306690 | 69722 | 14: 78312623 | 69725 | 14: 78390822 | 69726 | 14: 78392259 | 69727 | 14: 78393807 |
| 69729 | 14: 78454550 | 69738 | 14: 78651480 | 69747 | 14: 78858840 | 69755 | 14: 79091368 | 69781 | 14: 79790786 |
| 69787 | 14: 79936016 | 69795 | 14: 80077176 | 69796 | 14: 80078158 | 69809 | 14: 80313052 | 69832 | 14: 80855743 |
| 69848 | 14: 81221549 | 69849 | 14: 81264509 | 69850 | 14: 81267100 | 69851 | 14: 81267190 | 69852 | 14: 81267746 |
| 69853 | 14: 81295824 | 69854 | 14: 81295958 | 69861 | 14: 81372244 | 69862 | 14: 81379555 | 69863 | 14: 81392217 |
| 69872 | 14: 81555021 | 69873 | 14: 81583416 | 69876 | 14: 81598835 | 69879 | 14: 81634869 | 69881 | 14: 81683735 |
| 69882 | 14: 81698012 | 69883 | 14: 81701580 | 69887 | 14: 81727910 | 69893 | 14: 81835309 | 69895 | 14: 81857929 |
| 69897 | 14: 81940278 | 69899 | 14: 81972242 | 69903 | 14: 82015406 | 69904 | 14: 82016702 | 69905 | 14: 82026443 |
| 69909 | 14: 82091345 | 69933 | 15: 556424 | 69946 | 15: 1148939 | 69948 | 15: 1192605 | 69953 | 15: 1229858 |
| 69961 | 15: 1387083 | 69962 | 15: 1439384 | 70009 | 15: 2844618 | 70063 | 15: 3928859 | 70079 | 15: 4264863 |
| 70083 | 15: 4294447 | 70092 | 15: 4497670 | 70099 | 15: 4629275 | 70119 | 15: 4985484 | 70122 | 15: 5160844 |
| 70123 | 15: 5160845 | 70124 | 15: 5160859 | 70125 | 15: 5160876 | 70126 | 15: 5160956 | 70128 | 15: 5219937 |
| 70129 | 15: 5234380 | 70130 | 15: 5296961 | 70282 | 15: 5477594 | 70286 | 15: 5550405 | 70289 | 15: 5646426 |
| 70292 | 15: 5686808 | 70293 | 15: 5686891 | 70294 | 15: 5686893 | 70297 | 15: 5779163 | 70300 | 15: 5933216 |
| 70306 | 15: 6041257 | 70320 | 15: 6451524 | 70323 | 15: 6555645 | 70324 | 15: 6579715 | 70329 | 15: 6707584 |
| 70374 | 15: 7579129 | 70391 | 15: 8102324 | 70413 | 15: 8544147 | 70414 | 15: 8547793 | 70425 | 15: 8919907 |
| 70434 | 15: 9119501 | 70444 | 15: 9307296 | 70445 | 15: 9307388 | 70446 | 15: 9308875 | 70449 | 15: 9342376 |
| 70461 | 15: 9653267 | 70462 | 15: 9653311 | 70466 | 15: 9752248 | 70491 | 15: 10716741 | 70493 | 15: 10740715 |
| 70496 | 15: 10827424 | 70505 | 15: 11236303 | 70520 | 15: 11770301 | 70529 | 15: 12167647 | 70540 | 15: 12557589 |
| 70574 | 15: 13886436 | 70586 | 15: 14183988 | 70588 | 15: 14191992 | 70591 | 15: 14203309 | 70594 | 15: 14260966 |
| 70599 | 15: 14448696 | 70600 | 15: 14456572 | 70601 | 15: 14458763 | 70602 | 15: 14459519 | 70603 | 15: 14461707 |
| 70604 | 15: 14462057 | 70605 | 15: 14462078 | 70606 | 15: 14463666 | 70612 | 15: 14705603 | 70617 | 15: 14911197 |
| 70623 | 15: 15068168 | 70624 | 15: 15068209 | 70631 | 15: 15245724 | 70643 | 15: 15417315 | 70644 | 15: 15417341 |
| 70645 | 15: 15417537 | 70646 | 15: 15417584 | 70648 | 15: 15474272 | 70649 | 15: 15505264 | 70651 | 15: 15537455 |
| 70656 | 15: 15691423 | 70657 | 15: 15691442 | 70658 | 15: 15715228 | 70661 | 15: 15789950 | 70664 | 15: 15846394 |
| 70665 | 15: 15857275 | 70666 | 15: 15867538 | 70667 | 15: 15882003 | 70668 | 15: 15887057 | 70671 | 15: 15896498 |
| 70672 | 15: 15896623 | 70673 | 15: 15896626 | 70676 | 15: 15899461 | 70678 | 15: 15941852 | 70685 | 15: 16025726 |
| 70686 | 15: 16034972 | 70688 | 15: 16066600 | 70689 | 15: 16070995 | 70690 | 15: 16086621 | 70691 | 15: 16086825 |
| 70692 | 15: 16090802 | 70693 | 15: 16095590 | 70694 | 15: 16098140 | 70697 | 15: 16106288 | 70698 | 15: 16106299 |
| 70699 | 15: 16106352 | 70700 | 15: 16106429 | 70701 | 15: 16106956 | 70703 | 15: 16181648 | 70720 | 15: 16783559 |
| 70722 | 15: 16876160 | 70725 | 15: 17015197 | 70726 | 15: 17021701 | 70727 | 15: 17023074 | 70728 | 15: 17023080 |
| 70729 | 15: 17035605 | 70730 | 15: 17045413 | 70731 | 15: 17050219 | 70732 | 15: 17051620 | 70734 | 15: 17079996 |
| 70737 | 15: 17110054 | 70739 | 15: 17151448 | 70740 | 15: 17156993 | 70742 | 15: 17174949 | 70743 | 15: 17174997 |
| 70744 | 15: 17178030 | 70748 | 15: 17216169 | 70749 | 15: 17225710 | 70752 | 15: 17272317 | 70771 | 15: 17881167 |
| 70772 | 15: 17905884 | 70773 | 15: 17907768 | 70774 | 15: 17907803 | 70776 | 15: 17984528 | 70780 | 15: 18132345 |
| 70788 | 15: 18340406 | 70789 | 15: 18340444 | 70790 | 15: 18340452 | 70791 | 15: 18340495 | 70792 | 15: 18340522 |
| 70800 | 15: 18582994 | 70828 | 15: 19344195 | 70852 | 15: 20040778 | 70853 | 15: 20041510 | 70855 | 15: 20059908 |
| 70861 | 15: 20159086 | 70862 | 15: 20208131 | 70863 | 15: 20239028 | 70864 | 15: 20251395 | 70874 | 15: 20506266 |
| 70898 | 15: 20943851 | 70900 | 15: 20983899 | 70904 | 15: 21079829 | 70909 | 15: 21144577 | 70910 | 15: 21222779 |
| 70915 | 15: 21328250 | 70916 | 15: 21331201 | 70917 | 15: 21344247 | 70927 | 15: 21713133 | 70928 | 15: 21715125 |
| 70930 | 15: 21724458 | 70937 | 15: 21944261 | 70938 | 15: 21955334 | 70939 | 15: 21959233 | 70940 | 15: 21980185 |
| 70944 | 15: 22109125 | 70947 | 15: 22219010 | 70950 | 15: 22224493 | 70951 | 15: 22255512 | 70959 | 15: 22397563 |
| 70988 | 15: 23032442 | 70992 | 15: 23140676 | 70995 | 15: 23230388 | 71002 | 15: 23361381 | 71003 | 15: 23382217 |
| 71006 | 15: 23428462 | 71007 | 15: 23428524 | 71008 | 15: 23428581 | 71009 | 15: 23495909 | 71023 | 15: 23729866 |
| 71024 | 15: 23732149 | 71059 | 15: 23865919 | 71072 | 15: 24238065 | 71074 | 15: 24278519 | 71083 | 15: 24394620 |
| 71084 | 15: 24406959 | 71103 | 15: 24778258 | 71104 | 15: 24778597 | 71105 | 15: 24783092 | 71145 | 15: 25774695 |
| 71150 | 15: 25820329 | 71157 | 15: 25920672 | 71166 | 15: 26130885 | 71170 | 15: 26196504 | 71175 | 15: 26276504 |
| 71176 | 15: 26297137 | 71192 | 15: 26653665 | 71199 | 15: 26809489 | 71211 | 15: 27151330 | 71219 | 15: 27430624 |
| 71223 | 15: 27474421 | 71226 | 15: 27503920 | 71228 | 15: 27522579 | 71230 | 15: 27581168 | 71236 | 15: 27795197 |
| 71240 | 15: 27909371 | 71243 | 15: 27975706 | 71246 | 15: 28011102 | 71247 | 15: 28029857 | 71248 | 15: 28029959 |
| 71249 | 15: 28047023 | 71251 | 15: 28062942 | 71256 | 15: 28137955 | 71257 | 15: 28138113 | 71260 | 15: 28217846 |
| 71261 | 15: 28233069 | 71264 | 15: 28261207 | 71276 | 15: 28502744 | 71285 | 15: 28668348 | 71286 | 15: 28669780 |
| 71287 | 15: 28674486 | 71288 | 15: 28675386 | 71289 | 15: 28678307 | 71290 | 15: 28679179 | 71292 | 15: 28683279 |
| 71293 | 15: 28687052 | 71294 | 15: 28689278 | 71304 | 15: 28889609 | 71305 | 15: 28903033 | 71306 | 15: 28903036 |
| 71307 | 15: 28904856 | 71311 | 15: 28935265 | 71314 | 15: 28955129 | 71315 | 15: 28957203 | 71316 | 15: 28995849 |
| 71318 | 15: 29029067 | 71320 | 15: 29051391 | 71327 | 15: 29217795 | 71331 | 15: 29377994 | 71338 | 15: 29599939 |
| 71341 | 15: 29669861 | 71342 | 15: 29691482 | 71343 | 15: 29695545 | 71344 | 15: 29708525 | 71358 | 15: 30156144 |
| 71359 | 15: 30182678 | 71372 | 15: 30495429 | 71376 | 15: 30550572 | 71378 | 15: 30571590 | 71379 | 15: 30592484 |
| 71381 | 15: 30681136 | 71387 | 15: 30887853 | 71395 | 15: 31076884 | 71398 | 15: 31119240 | 71423 | 15: 31752349 |
| 71426 | 15: 31762344 | 71427 | 15: 31762695 | 71428 | 15: 31762716 | 71429 | 15: 31762815 | 71430 | 15: 31766858 |
| 71431 | 15: 31772217 | 71432 | 15: 31772367 | 71433 | 15: 31772508 | 71435 | 15: 31789881 | 71439 | 15: 31967339 |
| 71447 | 15: 32184560 | 71448 | 15: 32184561 | 71455 | 15: 32326526 | 71457 | 15: 32408675 | 71458 | 15: 32415848 |
| 71473 | 15: 32775518 | 71475 | 15: 32857841 | 71479 | 15: 32885957 | 71481 | 15: 32912616 | 71482 | 15: 32919523 |
| 71487 | 15: 33030880 | 71493 | 15: 33140018 | 71495 | 15: 33140162 | 71496 | 15: 33143682 | 71499 | 15: 33200022 |
| 71502 | 15: 33313146 | 71505 | 15: 33357725 | 71513 | 15: 33683094 | 71522 | 15: 33855059 | 71546 | 15: 34227669 |
| 71554 | 15: 34378606 | 71558 | 15: 34416916 | 71563 | 15: 34514270 | 71564 | 15: 34527315 | 71572 | 15: 34677976 |
| 71574 | 15: 34706081 | 71576 | 15: 34775584 | 71578 | 15: 34801602 | 71582 | 15: 34866384 | 71583 | 15: 34892433 |
| 71588 | 15: 34974291 | 71591 | 15: 35040731 | 71592 | 15: 35049691 | 71598 | 15: 35277152 | 71599 | 15: 35337946 |
| 71604 | 15: 35461378 | 71611 | 15: 35646977 | 71612 | 15: 35648952 | 71615 | 15: 35707211 | 71617 | 15: 35715226 |
| 71635 | 15: 36132660 | 71636 | 15: 36169165 | 71639 | 15: 36183126 | 71640 | 15: 36219316 | 71645 | 15: 36321976 |
| 71655 | 15: 36576380 | 71657 | 15: 36603077 | 71678 | 15: 37088135 | 71684 | 15: 37186172 | 71706 | 15: 37547916 |
| 71731 | 15: 38265442 | 71738 | 15: 38472609 | 71769 | 15: 39119041 | 71776 | 15: 39333095 | 71777 | 15: 39347738 |
| 71781 | 15: 39412550 | 71785 | 15: 39545268 | 71799 | 15: 39871286 | 71804 | 15: 39927546 | 71805 | 15: 39927550 |
| 71806 | 15: 39927601 | 71809 | 15: 40067276 | 71812 | 15: 40162512 | 71817 | 15: 40279882 | 71818 | 15: 40279893 |
| 71819 | 15: 40279894 | 71820 | 15: 40280012 | 71823 | 15: 40449461 | 71824 | 15: 40449484 | 71826 | 15: 40502987 |
| 71832 | 15: 40632071 | 71833 | 15: 40641136 | 71835 | 15: 40723287 | 71837 | 15: 40759558 | 71838 | 15: 40847130 |
| 71845 | 15: 40955841 | 71849 | 15: 41014898 | 71850 | 15: 41016306 | 71852 | 15: 41150753 | 71860 | 15: 41290689 |
| 71869 | 15: 41440324 | 71877 | 15: 41698377 | 71878 | 15: 41698492 | 71879 | 15: 41698511 | 71880 | 15: 41705974 |
| 71881 | 15: 41705975 | 71882 | 15: 41706097 | 71885 | 15: 41765753 | 71886 | 15: 41767083 | 71887 | 15: 41768163 |
| 71889 | 15: 41814640 | 71901 | 15: 42032808 | 71904 | 15: 42124927 | 71907 | 15: 42185672 | 71909 | 15: 42231274 |
| 71912 | 15: 42264995 | 71924 | 15: 42509261 | 71926 | 15: 42590389 | 71932 | 15: 42752352 | 71934 | 15: 42787098 |
| 71936 | 15: 42818617 | 71939 | 15: 42848256 | 71940 | 15: 42854523 | 71951 | 15: 43042213 | 71952 | 15: 43045041 |
| 71953 | 15: 43046535 | 71954 | 15: 43049707 | 71955 | 15: 43053427 | 71956 | 15: 43055412 | 71957 | 15: 43056476 |
| 71958 | 15: 43057569 | 71959 | 15: 43058822 | 71960 | 15: 43059592 | 71962 | 15: 43063078 | 71963 | 15: 43063710 |
| 71964 | 15: 43097237 | 71965 | 15: 43098155 | 71970 | 15: 43259917 | 71971 | 15: 43260963 | 71972 | 15: 43262025 |
| 71973 | 15: 43262890 | 71974 | 15: 43269560 | 71975 | 15: 43276395 | 71976 | 15: 43286382 | 71977 | 15: 43288762 |
| 71978 | 15: 43292368 | 71979 | 15: 43293082 | 71980 | 15: 43294086 | 71989 | 15: 43476234 | 71993 | 15: 43560696 |
| 71994 | 15: 43561701 | 71995 | 15: 43563456 | 71996 | 15: 43566385 | 71997 | 15: 43573222 | 71998 | 15: 43580011 |
| 72000 | 15: 43625295 | 72002 | 15: 43642580 | 72003 | 15: 43664227 | 72007 | 15: 43741512 | 72020 | 15: 43936008 |
| 72022 | 15: 43939348 | 72023 | 15: 43939353 | 72024 | 15: 43939488 | 72046 | 15: 44460580 | 72047 | 15: 44479177 |
| 72056 | 15: 44643217 | 72058 | 15: 44735861 | 72075 | 15: 45129015 | 72083 | 15: 45210490 | 72084 | 15: 45221891 |
| 72085 | 15: 45246954 | 72088 | 15: 45346073 | 72090 | 15: 45379221 | 72091 | 15: 45415112 | 72103 | 15: 45716064 |
| 72104 | 15: 45722551 | 72109 | 15: 45827220 | 72126 | 15: 46426137 | 72128 | 15: 46464105 | 72131 | 15: 46490284 |
| 72133 | 15: 46492960 | 72134 | 15: 46494185 | 72135 | 15: 46500386 | 72136 | 15: 46500459 | 72137 | 15: 46500629 |
| 72138 | 15: 46500752 | 72139 | 15: 46505669 | 72140 | 15: 46506092 | 72141 | 15: 46506887 | 72142 | 15: 46506889 |
| 72143 | 15: 46506939 | 72144 | 15: 46507314 | 72145 | 15: 46507929 | 72146 | 15: 46508001 | 72147 | 15: 46508021 |
| 72148 | 15: 46512024 | 72149 | 15: 46512113 | 72156 | 15: 46613300 | 72159 | 15: 46636679 | 72161 | 15: 46645269 |
| 72162 | 15: 46646092 | 72173 | 15: 46864410 | 72174 | 15: 46866871 | 72178 | 15: 46967802 | 72189 | 15: 47187912 |
| 72190 | 15: 47188138 | 72191 | 15: 47188507 | 72201 | 15: 47615122 | 72202 | 15: 47641029 | 72205 | 15: 47779635 |
| 72206 | 15: 47779640 | 72207 | 15: 47779664 | 72208 | 15: 47779674 | 72209 | 15: 47787011 | 72210 | 15: 47787118 |
| 72211 | 15: 47848072 | 72212 | 15: 47856278 | 72213 | 15: 47857368 | 72215 | 15: 47862235 | 72216 | 15: 47862345 |
| 72217 | 15: 47867091 | 72222 | 15: 48008735 | 72224 | 15: 48040175 | 72225 | 15: 48040515 | 72226 | 15: 48040954 |
| 72228 | 15: 48078030 | 72229 | 15: 48078755 | 72230 | 15: 48102626 | 72231 | 15: 48102703 | 72232 | 15: 48103271 |
| 72233 | 15: 48103402 | 72250 | 15: 48373937 | 72251 | 15: 48378524 | 72252 | 15: 48386761 | 72253 | 15: 48390883 |
| 72254 | 15: 48396110 | 72255 | 15: 48397484 | 72266 | 15: 48867306 | 72267 | 15: 48952912 | 72275 | 15: 49295120 |
| 72287 | 15: 49686635 | 72289 | 15: 49798310 | 72292 | 15: 49812025 | 72296 | 15: 50073269 | 72302 | 15: 50348347 |
| 72305 | 15: 50405124 | 72306 | 15: 50418732 | 72311 | 15: 50513682 | 72312 | 15: 50513924 | 72326 | 15: 51057213 |
| 72329 | 15: 51193044 | 72332 | 15: 51271630 | 72333 | 15: 51275342 | 72336 | 15: 51360447 | 72337 | 15: 51360592 |
| 72338 | 15: 51362026 | 72339 | 15: 51362043 | 72340 | 15: 51362061 | 72341 | 15: 51362080 | 72342 | 15: 51362276 |
| 72343 | 15: 51362277 | 72344 | 15: 51362431 | 72345 | 15: 51362457 | 72346 | 15: 51362458 | 72347 | 15: 51364116 |
| 72348 | 15: 51364137 | 72349 | 15: 51420115 | 72350 | 15: 51441064 | 72351 | 15: 51441095 | 72352 | 15: 51511445 |
| 72354 | 15: 51518000 | 72355 | 15: 51519061 | 72357 | 15: 51521753 | 72358 | 15: 51523084 | 72359 | 15: 51529172 |
| 72360 | 15: 51530078 | 72361 | 15: 51535001 | 72362 | 15: 51542928 | 72366 | 15: 51558105 | 72367 | 15: 51563195 |
| 72370 | 15: 51568569 | 72371 | 15: 51576564 | 72372 | 15: 51610810 | 72376 | 15: 51684740 | 72377 | 15: 51687709 |
| 72379 | 15: 51689738 | 72382 | 15: 51736976 | 72384 | 15: 51790467 | 72386 | 15: 51803577 | 72394 | 15: 51988641 |
| 72397 | 15: 52007899 | 72404 | 15: 52185081 | 72406 | 15: 52200879 | 72413 | 15: 52360124 | 72424 | 15: 52658762 |
| 72425 | 15: 52660106 | 72426 | 15: 52673691 | 72441 | 15: 53067270 | 72445 | 15: 53137213 | 72449 | 15: 53308023 |
| 72451 | 15: 53309880 | 72452 | 15: 53310424 | 72453 | 15: 53311034 | 72454 | 15: 53311897 | 72455 | 15: 53312462 |
| 72456 | 15: 53314094 | 72457 | 15: 53314682 | 72458 | 15: 53316670 | 72459 | 15: 53319540 | 72460 | 15: 53320533 |
| 72461 | 15: 53321307 | 72462 | 15: 53323633 | 72463 | 15: 53324367 | 72465 | 15: 53325750 | 72466 | 15: 53326273 |
| 72467 | 15: 53326849 | 72468 | 15: 53327898 | 72469 | 15: 53332122 | 72471 | 15: 53333616 | 72472 | 15: 53338788 |
| 72476 | 15: 53398368 | 72478 | 15: 53441398 | 72486 | 15: 53709788 | 7248 | 15: 53727634 | 72488 | 15: 53729356 |
| 72489 | 15: 53738936 | 72490 | 15: 53743609 | 72491 | 15: 53753517 | 72492 | 15: 53761189 | 72494 | 15: 53788777 |
| 72495 | 15: 53796595 | 72496 | 15: 53800970 | 72502 | 15: 53945334 | 72518 | 15: 54255643 | 72527 | 15: 54462755 |
| 72530 | 15: 54489427 | 72531 | 15: 54500400 | 72541 | 15: 54748501 | 72544 | 15: 54834057 | 72548 | 15: 54885008 |
| 72549 | 15: 54885114 | 72550 | 15: 54885117 | 72551 | 15: 54889719 | 72552 | 15: 54889820 | 72556 | 15: 55119302 |
| 72565 | 15: 55289790 | 72569 | 15: 55444251 | 72574 | 15: 55529280 | 72582 | 15: 55734507 | 72589 | 15: 56022102 |
| 72596 | 15: 56200579 | 72603 | 15: 56497282 | 72604 | 15: 56497450 | 72605 | 15: 56501007 | 72607 | 15: 56515635 |
| 72608 | 15: 56515639 | 72609 | 15: 56515642 | 72610 | 15: 56515714 | 72611 | 15: 56518845 | 72612 | 15: 56518860 |
| 72613 | 15: 56518880 | 72614 | 15: 56518892 | 72615 | 15: 56518948 | 72616 | 15: 56519916 | 72617 | 15: 56520003 |
| 72618 | 15: 56520009 | 72619 | 15: 56522354 | 72620 | 15: 56523085 | 72621 | 15: 56523145 | 72622 | 15: 56525703 |
| 72623 | 15: 56526321 | 72624 | 15: 56527735 | 72625 | 15: 56529668 | 72626 | 15: 56548649 | 72644 | 15: 56809212 |
| 72675 | 15: 57003970 | 72684 | 15: 57272924 | 72689 | 15: 57423300 | 72692 | 15: 57500020 | 72694 | 15: 57536234 |
| 72695 | 15: 57536250 | 72703 | 15: 57718142 | 72709 | 15: 57803493 | 72712 | 15: 57854603 | 72722 | 15: 58001428 |
| 72727 | 15: 58088362 | 72734 | 15: 58241375 | 72736 | 15: 58282071 | 72737 | 15: 58283033 | 72740 | 15: 58330257 |
| 72760 | 15: 58671427 | 72773 | 15: 59112131 | 72787 | 15: 59537850 | 72795 | 15: 59796390 | 72809 | 15: 60285268 |
| 72813 | 15: 60368718 | 72815 | 15: 60428854 | 72820 | 15: 60523980 | 72823 | 15: 60608578 | 72850 | 15: 61257843 |
| 72854 | 15: 61342496 | 72868 | 15: 61603248 | 72869 | 15: 61639615 | 72871 | 15: 61721710 | 72872 | 15: 61721767 |
| 72873 | 15: 61724284 | 72874 | 15: 61724369 | 72875 | 15: 61724392 | 72876 | 15: 61725702 | 72879 | 15: 61767375 |
| 72894 | 15: 62032399 | 72907 | 15: 62467385 | 72912 | 15: 62561316 | 72914 | 15: 62563939 | 72915 | 15: 62586048 |
| 72916 | 15: 62586078 | 72920 | 15: 62652293 | 72938 | 15: 63066650 | 72940 | 15: 63100087 | 72941 | 15: 63107739 |
| 72949 | 15: 63295827 | 72951 | 15: 63300815 | 72952 | 15: 63303147 | 72960 | 15: 63529465 | 72962 | 15: 63574887 |
| 72964 | 15: 63579300 | 72969 | 15: 63631630 | 72972 | 15: 63722937 | 72973 | 15: 63723070 | 72974 | 15: 63743524 |
| 72979 | 15: 63917098 | 72983 | 15: 63967865 | 72997 | 15: 64256628 | 72999 | 15: 64283258 | 73001 | 15: 64333153 |
| 73004 | 15: 64416446 | 73023 | 15: 64714146 | 73026 | 15: 64744225 | 73027 | 15: 64771598 | 73031 | 15: 64801402 |
| 73033 | 15: 64806923 | 73038 | 15: 64854504 | 73040 | 15: 64908292 | 73046 | 15: 64986216 | 73050 | 15: 65029608 |
| 73056 | 15: 65148971 | 73064 | 15: 65275286 | 73066 | 15: 65335638 | 73075 | 15: 65436899 | 73078 | 15: 65521232 |
| 73080 | 15: 65594811 | 73081 | 15: 65607689 | 73083 | 15: 65622127 | 73085 | 15: 65656084 | 73086 | 15: 65670390 |
| 73091 | 15: 65822176 | 73093 | 15: 65854403 | 73105 | 15: 66145675 | 73106 | 15: 66156197 | 73115 | 15: 66337078 |
| 73117 | 15: 66339107 | 73119 | 15: 66380166 | 73121 | 15: 66418487 | 73124 | 15: 66460251 | 73127 | 15: 66496173 |
| 73129 | 15: 66543651 | 73141 | 15: 66724079 | 73146 | 15: 66881298 | 73154 | 15: 67179102 | 73164 | 15: 67470477 |
| 73176 | 15: 67880702 | 73196 | 15: 68313970 | 73199 | 15: 68378183 | 73216 | 15: 68982890 | 73217 | 15: 69062551 |
| 73224 | 15: 69173709 | 73249 | 15: 70010700 | 73255 | 15: 70308624 | 73259 | 15: 70429364 | 73262 | 15: 70465643 |
| 73282 | 15: 71042092 | 73289 | 15: 71280393 | 73299 | 15: 71468201 | 73300 | 15: 71468245 | 73315 | 15: 71909279 |
| 73349 | 15: 72924654 | 73350 | 15: 72992522 | 73355 | 15: 73041310 | 73358 | 15: 73097589 | 73360 | 15: 73124963 |
| 73362 | 15: 73143263 | 73363 | 15: 73144008 | 73364 | 15: 73144009 | 73365 | 15: 73144095 | 73366 | 15: 73144112 |
| 73367 | 15: 73144117 | 73368 | 15: 73144142 | 73372 | 15: 73231049 | 73373 | 15: 73250550 | 73376 | 15: 73270606 |
| 73385 | 15: 73422749 | 73386 | 15: 73470922 | 73389 | 15: 73555524 | 73390 | 15: 73555544 | 73391 | 15: 73555546 |
| 73392 | 15: 73555549 | 73394 | 15: 73590486 | 73400 | 15: 73747012 | 73407 | 15: 74016858 | 73408 | 15: 74019876 |
| 73409 | 15: 74047524 | 73412 | 15: 74101712 | 73413 | 15: 74120008 | 73421 | 15: 74204147 | 73422 | 15: 74221807 |
| 73423 | 15: 74236733 | 73440 | 15: 74680300 | 73442 | 15: 74706685 | 73443 | 15: 74715602 | 73444 | 15: 74730746 |
| 73445 | 15: 74736252 | 73448 | 15: 74763309 | 73449 | 15: 74765962 | 73456 | 15: 74857238 | 73476 | 15: 75401306 |
| 73484 | 15: 75520621 | 73496 | 15: 75764247 | 73498 | 15: 75807751 | 73505 | 15: 76018207 | 73513 | 15: 76268285 |
| 73514 | 15: 76284024 | 73515 | 15: 76295386 | 73517 | 15: 76357310 | 73519 | 15: 76369909 | 73520 | 15: 76423356 |
| 73525 | 15: 76490114 | 73530 | 15: 76627029 | 73531 | 15: 76628527 | 73533 | 15: 76676572 | 73542 | 15: 76909234 |
| 73543 | 15: 76913709 | 73554 | 15: 77213130 | 73558 | 15: 77256270 | 73561 | 15: 77333647 | 73563 | 15: 77341921 |
| 73569 | 15: 77457892 | 73570 | 15: 77533291 | 73572 | 15: 77565837 | 73573 | 15: 77581726 | 73574 | 15: 77581747 |
| 73575 | 15: 77581752 | 73576 | 15: 77581872 | 73577 | 15: 77581882 | 73580 | 15: 77638946 | 73582 | 15: 77676086 |
| 73590 | 15: 77841257 | 73591 | 15: 77844974 | 73597 | 15: 77988330 | 73598 | 15: 77989577 | 73600 | 15: 77997353 |
| 73601 | 15: 78001161 | 73603 | 15: 78001840 | 73604 | 15: 78003499 | 73605 | 15: 78004209 | 73606 | 15: 78008228 |
| 73607 | 15: 78010723 | 73608 | 15: 78011919 | 73610 | 15: 78017138 | 73613 | 15: 78027580 | 73614 | 15: 78028904 |
| 73615 | 15: 78032066 | 73616 | 15: 78033166 | 73617 | 15: 78033926 | 73618 | 15: 78036014 | 73619 | 15: 78037096 |
| 73620 | 15: 78043902 | 73621 | 15: 78049025 | 73623 | 15: 78065440 | 73624 | 15: 78069489 | 73637 | 15: 78497070 |
| 73643 | 15: 78705678 | 73644 | 15: 78711863 | 73654 | 15: 79013310 | 73662 | 15: 79316918 | 73663 | 15: 79316919 |
| 73665 | 15: 79446531 | 73666 | 15: 79447076 | 73667 | 15: 79447126 | 73668 | 15: 79447145 | 73671 | 15: 79651229 |
| 73672 | 15: 79651253 | 73684 | 15: 79994534 | 73699 | 15: 80313710 | 73702 | 15: 80329440 | 73707 | 15: 80387982 |
| 73711 | 15: 80447483 | 73714 | 15: 80528280 | 73722 | 15: 80779796 | 73724 | 15: 80825322 | 73725 | 15: 80846525 |
| 73728 | 15: 80988789 | 73733 | 15: 81116878 | 73735 | 15: 81144733 | 73736 | 15: 81149287 | 73737 | 15: 81161372 |
| 73739 | 15: 81168190 | 73744 | 15: 81197998 | 73751 | 15: 81267470 | 73752 | 15: 81279270 | 73759 | 15: 81372132 |
| 73775 | 15: 81857983 | 73793 | 15: 82380481 | 73799 | 15: 82629156 | 73806 | 15: 82720061 | 73807 | 15: 82720089 |
| 73810 | 15: 82745967 | 73811 | 15: 82746237 | 73815 | 15: 82788081 | 73817 | 15: 82797551 | 73819 | 15: 82843451 |
| 73820 | 15: 82851394 | 73824 | 15: 82933219 | 73825 | 15: 82949122 | 73830 | 15: 83006747 | 73831 | 15: 83013517 |
| 73832 | 15: 83013534 | 73840 | 15: 83073328 | 73848 | 15: 83277486 | 73849 | 15: 83288242 | 73850 | 15: 83309084 |
| 73855 | 15: 83458586 | 73865 | 15: 83567476 | 73866 | 15: 83583523 | 73867 | 15: 83586864 | 73868 | 15: 83586930 |
| 73911 | 16: 752827 | 73914 | 16: 766023 | 73915 | 16: 804997 | 73916 | 16: 806537 | 73922 | 16: 921175 |
| 73929 | 16: 978613 | 73937 | 16: 1181761 | 73939 | 16: 1204788 | 73941 | 16: 1214929 | 73946 | 16: 1324936 |
| 73963 | 16: 1582068 | 73964 | 16: 1599816 | 73965 | 16: 1625990 | 73988 | 16: 2055227 | 73992 | 16: 2194637 |
| 73993 | 16: 2224824 | 73995 | 16: 2248476 | 73996 | 16: 2263389 | 73997 | 16: 2277642 | 74004 | 16: 2402507 |
| 74005 | 16: 2467947 | 74013 | 16: 2602159 | 74017 | 16: 2743842 | 74023 | 16: 2938620 | 74026 | 16: 3018942 |
| 74030 | 16: 3067157 | 74038 | 16: 3201109 | 74040 | 16: 3201263 | 74042 | 16: 3253128 | 74043 | 16: 3269182 |
| 74044 | 16: 3269280 | 74045 | 16: 3270297 | 74051 | 16: 3378716 | 74055 | 16: 3405375 | 74059 | 16: 3465967 |
| 74062 | 16: 3607615 | 74065 | 16: 3643356 | 74066 | 16: 3644483 | 74073 | 16: 3791803 | 74080 | 16: 3992289 |
| 74087 | 16: 4106339 | 74088 | 16: 4109307 | 74093 | 16: 4171303 | 74099 | 16: 4262626 | 74100 | 16: 4262650 |
| 74101 | 16: 4265692 | 74103 | 16: 4290878 | 74109 | 16: 4423692 | 74114 | 16: 4475911 | 74119 | 16: 4592258 |
| 74122 | 16: 4640118 | 74125 | 16: 4717716 | 74129 | 16: 4872430 | 74130 | 16: 4898040 | 74133 | 16: 4999290 |
| 74140 | 16: 5116140 | 74146 | 16: 5243133 | 74147 | 16: 5265259 | 74148 | 16: 5265363 | 74162 | 16: 6052639 |
| 74164 | 16: 6060113 | 74166 | 16: 6207254 | 74168 | 16: 6308221 | 74169 | 16: 6317267 | 74170 | 16: 6318112 |
| 74171 | 16: 6319483 | 74172 | 16: 6323633 | 74173 | 16: 6325179 | 74175 | 16: 6332032 | 74176 | 16: 6333222 |
| 74177 | 16: 6335935 | 74180 | 16: 6376238 | 74191 | 16: 6749567 | 74204 | 16: 7268343 | 74214 | 16: 7503961 |
| 74270 | 16: 9734287 | 74280 | 16: 9996877 | 74307 | 16: 10894806 | 74318 | 16: 11281651 | 74336 | 16: 11756836 |
| 74343 | 16: 11874391 | 74347 | 16: 11985702 | 74364 | 16: 12483562 | 74365 | 16: 12486013 | 74378 | 16: 12756820 |
| 74383 | 16: 12870981 | 74400 | 16: 13072437 | 74411 | 16: 13341826 | 74415 | 16: 13479712 | 74431 | 16: 13921125 |
| 74441 | 16: 14274292 | 74444 | 16: 14375476 | 74450 | 16: 14512750 | 74456 | 16: 14612740 | 74457 | 16: 14612809 |
| 74485 | 16: 15352405 | 74486 | 16: 15355419 | 74514 | 16: 16201608 | 74519 | 16: 16433665 | 74520 | 16: 16433701 |
| 74532 | 16: 16902875 | 74586 | 16: 18664935 | 74596 | 16: 18957732 | 74624 | 16: 19622167 | 74629 | 16: 19786076 |
| 74645 | 16: 20175121 | 74649 | 16: 20294926 | 74659 | 16: 20540783 | 74660 | 16: 20555295 | 74674 | 16: 20905213 |
| 74676 | 16: 20943232 | 74715 | 16: 21954699 | 74722 | 16: 22188120 | 74724 | 16: 22316546 | 74726 | 16: 22390334 |
| 74729 | 16: 22420005 | 74742 | 16: 22743613 | 74744 | 16: 22775537 | 74745 | 16: 22786666 | 74762 | 16: 23286838 |
| 74768 | 16: 23427214 | 74769 | 16: 23439282 | 74770 | 16: 23508581 | 74771 | 16: 23518024 | 74772 | 16: 23544281 |
| 74773 | 16: 23557997 | 74776 | 16: 23621715 | 74782 | 16: 23717545 | 74783 | 16: 23721462 | 74792 | 16: 23915566 |
| 74793 | 16: 23918537 | 74801 | 16: 24174300 | 74802 | 16: 24198154 | 74806 | 16: 24253488 | 74809 | 16: 24321784 |
| 74822 | 16: 24770805 | 74844 | 16: 24996364 | 74847 | 16: 25007172 | 74856 | 16: 25057711 | 74879 | 16: 25209115 |
| 74900 | 16: 25434176 | 74908 | 16: 25723685 | 74909 | 16: 25753006 | 74910 | 16: 25760167 | 74911 | 16: 25845363 |
| 74915 | 16: 25999582 | 74916 | 16: 26009236 | 74917 | 16: 26012436 | 74918 | 16: 26021774 | 74920 | 16: 26036756 |
| 74921 | 16: 26037402 | 74924 | 16: 26139905 | 74925 | 16: 26150072 | 74926 | 16: 26150074 | 74927 | 16: 26153413 |
| 74929 | 16: 26186921 | 74930 | 16: 26216113 | 74931 | 16: 26217301 | 74933 | 16: 26220845 | 74934 | 16: 26227564 |
| 74935 | 16: 26274073 | 74936 | 16: 26296242 | 74940 | 16: 26352124 | 74941 | 16: 26356312 | 74943 | 16: 26451726 |
| 74951 | 16: 26637237 | 74973 | 16: 27186026 | 74989 | 16: 27756698 | 74996 | 16: 27880735 | 74997 | 16: 27880880 |
| 75002 | 16: 27952368 | 75003 | 16: 28008613 | 75004 | 16: 28008626 | 75013 | 16: 28274084 | 75024 | 16: 28665499 |
| 75034 | 16: 28945983 | 75036 | 16: 29003100 | 75057 | 16: 29692650 | 75059 | 16: 29709436 | 75060 | 16: 29760720 |
| 75061 | 16: 29760768 | 75064 | 16: 29872847 | 75065 | 16: 29893692 | 75079 | 16: 30287006 | 75081 | 16: 30348202 |
| 75088 | 16: 30604134 | 75092 | 16: 30729515 | 75096 | 16: 30772837 | 75098 | 16: 30794250 | 75099 | 16: 30795047 |
| 75100 | 16: 30802513 | 75102 | 16: 30839358 | 75108 | 16: 31046070 | 75109 | 16: 31054739 | 75120 | 16: 31360463 |
| 75130 | 16: 31587509 | 75131 | 16: 31613864 | 75137 | 16: 31734054 | 75146 | 16: 31961054 | 75147 | 16: 31961057 |
| 75151 | 16: 32067908 | 75157 | 16: 32356916 | 75162 | 16: 32428255 | 75165 | 16: 32550214 | 75180 | 16: 32851343 |
| 75183 | 16: 32875785 | 75184 | 16: 32880400 | 75187 | 16: 32951618 | 75188 | 16: 32951739 | 75215 | 16: 33649281 |
| 75216 | 16: 33650436 | 75220 | 16: 33885770 | 75224 | 16: 33965943 | 75226 | 16: 33984101 | 75247 | 16: 34572625 |
| 75250 | 16: 34620173 | 75254 | 16: 34722052 | 75257 | 16: 34852541 | 75262 | 16: 34933617 | 75263 | 16: 34933651 |
| 75267 | 16: 35001017 | 75270 | 16: 35137739 | 75272 | 16: 35194830 | 75273 | 16: 35194886 | 75274 | 16: 35194897 |
| 75276 | 16: 35202714 | 75277 | 16: 35234653 | 75278 | 16: 35244272 | 75279 | 16: 35254252 | 75280 | 16: 35290661 |
| 75281 | 16: 35295775 | 75295 | 16: 35792938 | 75302 | 16: 35995859 | 75303 | 16: 36000751 | 75304 | 16: 36004313 |
| 75307 | 16: 36081689 | 75313 | 16: 36290108 | 75314 | 16: 36299734 | 75317 | 16: 36326781 | 75321 | 16: 36365140 |
| 75330 | 16: 36566768 | 75335 | 16: 36697557 | 75336 | 16: 36712618 | 75337 | 16: 36775388 | 75344 | 16: 36945543 |
| 75345 | 16: 36969420 | 75365 | 16: 37448884 | 75366 | 16: 37448890 | 75376 | 16: 37796926 | 75381 | 16: 37913794 |
| 75382 | 16: 37913795 | 75383 | 16: 37913847 | 75384 | 16: 37913888 | 75386 | 16: 37937575 | 75397 | 16: 38107328 |
| 75399 | 16: 38122376 | 75401 | 16: 38137107 | 75402 | 16: 38150887 | 75406 | 16: 38234762 | 75407 | 16: 38244862 |
| 75423 | 16: 38532788 | 75431 | 16: 38885897 | 75458 | 16: 39922839 | 75459 | 16: 39929733 | 75482 | 16: 40598638 |
| 75485 | 16: 40704573 | 75500 | 16: 41266328 | 75513 | 16: 41835712 | 75514 | 16: 41852637 | 75515 | 16: 41881957 |
| 75516 | 16: 41882451 | 75517 | 16: 41882906 | 75518 | 16: 41882957 | 75520 | 16: 41883859 | 75521 | 16: 41884250 |
| 75522 | 16: 41889773 | 75523 | 16: 41890000 | 75524 | 16: 41892823 | 75525 | 16: 41896465 | 75526 | 16: 41898495 |
| 75527 | 16: 41898551 | 75528 | 16: 41908374 | 75529 | 16: 41908542 | 75530 | 16: 41909276 | 75531 | 16: 41909349 |
| 75532 | 16: 41909492 | 75533 | 16: 41909626 | 75534 | 16: 41909729 | 75535 | 16: 41909886 | 75536 | 16: 41929565 |
| 75537 | 16: 41931893 | 75538 | 16: 41931907 | 75539 | 16: 41934619 | 75540 | 16: 41934651 | 75541 | 16: 41934687 |
| 75543 | 16: 41947567 | 75544 | 16: 41964938 | 75554 | 16: 42253482 | 75557 | 16: 42465089 | 75558 | 16: 42547847 |
| 75559 | 16: 42600403 | 75569 | 16: 43061490 | 75575 | 16: 43278476 | 75576 | 16: 43330994 | 75578 | 16: 43398900 |
| 75583 | 16: 43745014 | 75593 | 16: 44139005 | 75604 | 16: 44403647 | 75606 | 16: 44524286 | 75609 | 16: 44660436 |
| 75610 | 16: 44759709 | 75625 | 16: 45375047 | 75641 | 16: 46123551 | 75647 | 16: 46250903 | 75649 | 16: 46275222 |
| 75650 | 16: 46286961 | 75651 | 16: 46287045 | 75658 | 16: 46502252 | 75665 | 16: 46683276 | 75671 | 16: 46787853 |
| 75672 | 16: 46830087 | 75680 | 16: 47040946 | 75682 | 16: 47083406 | 75684 | 16: 47094623 | 75685 | 16: 47120517 |
| 75689 | 16: 47196401 | 75697 | 16: 47379730 | 75698 | 16: 47379738 | 75702 | 16: 47529414 | 75709 | 16: 47649083 |
| 75710 | 16: 47653055 | 75711 | 16: 47658993 | 75719 | 16: 47876641 | 75748 | 16: 48471006 | 75749 | 16: 48471012 |
| 75750 | 16: 48471070 | 75767 | 16: 48815391 | 75788 | 16: 49290662 | 75798 | 16: 49608069 | 75800 | 16: 49640815 |
| 75804 | 16: 49736666 | 75816 | 16: 50023350 | 75856 | 16: 51315511 | 75862 | 16: 51572737 | 75866 | 16: 51638935 |
| 75870 | 16: 51814662 | 75871 | 16: 51818542 | 75872 | 16: 51823133 | 75873 | 16: 51827521 | 75874 | 16: 51828907 |
| 75875 | 16: 51830294 | 75876 | 16: 51830796 | 75877 | 16: 51841289 | 75879 | 16: 51854505 | 75881 | 16: 51899632 |
| 75889 | 16: 52062903 | 75892 | 16: 52124016 | 75896 | 16: 52189080 | 75897 | 16: 52203550 | 75905 | 16: 52391190 |
| 75906 | 16: 52391213 | 75907 | 16: 52401546 | 75908 | 16: 52413488 | 75911 | 16: 52492743 | 75920 | 16: 52664549 |
| 75922 | 16: 52715395 | 75933 | 16: 53089976 | 75945 | 16: 53432925 | 75951 | 16: 53742380 | 75952 | 16: 53742397 |
| 75953 | 16: 53742405 | 75954 | 16: 53742482 | 75960 | 16: 53807690 | 75962 | 16: 53819240 | 75971 | 16: 54022291 |
| 75972 | 16: 54037567 | 75973 | 16: 54047358 | 76003 | 16: 54893701 | 76004 | 16: 54916866 | 76005 | 16: 54917648 |
| 76008 | 16: 54974984 | 76009 | 16: 54975864 | 76010 | 16: 55000694 | 76011 | 16: 55015703 | 76012 | 16: 55021774 |
| 76013 | 16: 55109042 | 76032 | 16: 55624726 | 76041 | 16: 55875606 | 76053 | 16: 56173257 | 76055 | 16: 56193959 |
| 76056 | 16: 56206450 | 76066 | 16: 56367576 | 76067 | 16: 56372228 | 76069 | 16: 56383292 | 76072 | 16: 56444611 |
| 76077 | 16: 56546719 | 76080 | 16: 56571482 | 76081 | 16: 56571485 | 76082 | 16: 56571494 | 76089 | 16: 56837687 |
| 76096 | 16: 57122424 | 76100 | 16: 57326320 | 76101 | 16: 57329788 | 76113 | 16: 57586740 | 76114 | 16: 57608446 |
| 76126 | 16: 57873879 | 76129 | 16: 58011779 | 76130 | 16: 58012586 | 76146 | 16: 58359793 | 76156 | 16: 58464078 |
| 76158 | 16: 58524073 | 76175 | 16: 58904562 | 76176 | 16: 58904595 | 76199 | 16: 59323799 | 76201 | 16: 59330153 |
| 76211 | 16: 59665282 | 76212 | 16: 59689730 | 76213 | 16: 59689802 | 76222 | 16: 59946371 | 76231 | 16: 60201651 |
| 76233 | 16: 60224702 | 76234 | 16: 60224712 | 76236 | 16: 60251070 | 76237 | 16: 60253242 | 76238 | 16: 60254578 |
| 76240 | 16: 60311442 | 76241 | 16: 60322616 | 76242 | 16: 60322634 | 76245 | 16: 60357272 | 76247 | 16: 60391026 |
| 76251 | 16: 60558830 | 76258 | 16: 60784981 | 76274 | 16: 61120937 | 76279 | 16: 61322631 | 76285 | 16: 61383011 |
| 76286 | 16: 61387337 | 76301 | 16: 61731673 | 76306 | 16: 61957053 | 76312 | 16: 62071218 | 76313 | 16: 62072068 |
| 76314 | 16: 62072151 | 76315 | 16: 62072468 | 76316 | 16: 62077343 | 76318 | 16: 62094667 | 76319 | 16: 62094728 |
| 76327 | 16: 62245438 | 76328 | 16: 62267314 | 76329 | 16: 62275255 | 76335 | 16: 62455462 | 76343 | 16: 62633633 |
| 76348 | 16: 62852449 | 76349 | 16: 62856876 | 76354 | 16: 62916406 | 76356 | 16: 62926757 | 76369 | 16: 63226438 |
| 76370 | 16: 63226475 | 76371 | 16: 63252161 | 76372 | 16: 63264840 | 76374 | 16: 63283115 | 76376 | 16: 63353590 |
| 76382 | 16: 63498489 | 76389 | 16: 63665761 | 76391 | 16: 63691003 | 76395 | 16: 63806226 | 76397 | 16: 63832124 |
| 76403 | 16: 63910273 | 76404 | 16: 63918137 | 76406 | 16: 63985213 | 76413 | 16: 64162147 | 76421 | 16: 64386831 |
| 76426 | 16: 64477575 | 76427 | 16: 64478870 | 76428 | 16: 64480600 | 76429 | 16: 64482426 | 76430 | 16: 64483727 |
| 76431 | 16: 64486623 | 76432 | 16: 64488080 | 76434 | 16: 64560495 | 76437 | 16: 64585141 | 76438 | 16: 64587028 |
| 76439 | 16: 64599607 | 76447 | 16: 64716072 | 76457 | 16: 64911089 | 76458 | 16: 64915512 | 76459 | 16: 64917512 |
| 76460 | 16: 64917539 | 76461 | 16: 64920273 | 76462 | 16: 64924645 | 76463 | 16: 64945487 | 76464 | 16: 64947395 |
| 76466 | 16: 64979912 | 76467 | 16: 64979914 | 76468 | 16: 64979920 | 76469 | 16: 64980083 | 76473 | 16: 65100643 |
| 76479 | 16: 65256823 | 76480 | 16: 65263949 | 76483 | 16: 65320518 | 76490 | 16: 65492703 | 76491 | 16: 65500984 |
| 76492 | 16: 65508159 | 76493 | 16: 65511465 | 76494 | 16: 65514812 | 76495 | 16: 65518974 | 76496 | 16: 65521306 |
| 76505 | 16: 65841167 | 76512 | 16: 66020681 | 76515 | 16: 66100353 | 76517 | 16: 66121444 | 76521 | 16: 66169638 |
| 76523 | 16: 66271884 | 76532 | 16: 66520379 | 76533 | 16: 66528712 | 76536 | 16: 66553762 | 76538 | 16: 66566651 |
| 76550 | 16: 66804665 | 76551 | 16: 66825677 | 76553 | 16: 66859242 | 76555 | 16: 66896436 | 76557 | 16: 66919773 |
| 76558 | 16: 66919950 | 76564 | 16: 67050756 | 76565 | 16: 67108343 | 76571 | 16: 67259430 | 76573 | 16: 67296635 |
| 76580 | 16: 67413442 | 76581 | 16: 67414503 | 76582 | 16: 67414576 | 76583 | 16: 67414591 | 76584 | 16: 67418245 |
| 76588 | 16: 67495932 | 76599 | 16: 67610069 | 76610 | 16: 67976663 | 76611 | 16: 67981588 | 76626 | 16: 68435498 |
| 76627 | 16: 68435550 | 76636 | 16: 68624997 | 76652 | 16: 69066498 | 76653 | 16: 69066625 | 76656 | 16: 69080402 |
| 76657 | 16: 69086868 | 76660 | 16: 69129862 | 76665 | 16: 69230127 | 76671 | 16: 69350387 | 76679 | 16: 69497807 |
| 76680 | 16: 69501212 | 76681 | 16: 69541904 | 76685 | 16: 69607606 | 76688 | 16: 69676831 | 76689 | 16: 69704477 |
| 76692 | 16: 69802000 | 76719 | 16: 70380777 | 76720 | 16: 70381368 | 76730 | 16: 70587239 | 76731 | 16: 70590791 |
| 76732 | 16: 70590919 | 76733 | 16: 70608188 | 76744 | 16: 70871474 | 76754 | 16: 71132906 | 76759 | 16: 71385575 |
| 76762 | 16: 71432921 | 76766 | 16: 71483613 | 76771 | 16: 71716120 | 76772 | 16: 71732153 | 76777 | 16: 71834818 |
| 76784 | 16: 71964464 | 76785 | 16: 71974676 | 76792 | 16: 72212208 | 76797 | 16: 72370528 | 76798 | 16: 72370550 |
| 76799 | 16: 72370562 | 76800 | 16: 72370636 | 76803 | 16: 72382117 | 76804 | 16: 72382163 | 76805 | 16: 72382236 |
| 76806 | 16: 72382280 | 76808 | 16: 72394792 | 76811 | 16: 72421917 | 76813 | 16: 72440326 | 76814 | 16: 72473155 |
| 76815 | 16: 72473197 | 76821 | 16: 72619958 | 76823 | 16: 72642068 | 76826 | 16: 72684703 | 76828 | 16: 72684778 |
| 76829 | 16: 72684811 | 76831 | 16: 72698852 | 76832 | 16: 72700502 | 76833 | 16: 72701289 | 76848 | 16: 73201335 |
| 76853 | 16: 73303554 | 76860 | 16: 73466388 | 76862 | 16: 73504205 | 76865 | 16: 73518067 | 76866 | 16: 73534249 |
| 76868 | 16: 73563070 | 76869 | 16: 73563134 | 76871 | 16: 73583302 | 76873 | 16: 73591363 | 76874 | 16: 73597712 |
| 76876 | 16: 73599978 | 76877 | 16: 73607828 | 76878 | 16: 73622925 | 76879 | 16: 73694168 | 76880 | 16: 73694195 |
| 76881 | 16: 73694803 | 76882 | 16: 73697167 | 76883 | 16: 73697204 | 76884 | 16: 73697206 | 76885 | 16: 73697258 |
| 76886 | 16: 73697700 | 76887 | 16: 73699147 | 76889 | 16: 73715301 | 76892 | 16: 73750079 | 76909 | 16: 74262736 |
| 76910 | 16: 74267964 | 76924 | 16: 74548368 | 76931 | 16: 74775935 | 76936 | 16: 74853499 | 76945 | 16: 75005400 |
| 76946 | 16: 75005468 | 76948 | 16: 75032047 | 76953 | 16: 75118546 | 76954 | 16: 75132927 | 76955 | 16: 75142495 |
| 76956 | 16: 75143544 | 76957 | 16: 75153428 | 76959 | 16: 75202467 | 76960 | 16: 75217027 | 76963 | 16: 75248051 |
| 76965 | 16: 75255731 | 76966 | 16: 75256528 | 76967 | 16: 75273493 | 76971 | 16: 75346062 | 76978 | 16: 75476985 |
| 76979 | 16: 75477188 | 76980 | 16: 75487044 | 76986 | 16: 75656899 | 76991 | 16: 75736008 | 76996 | 16: 75817486 |
| 77008 | 16: 76064461 | 77010 | 16: 76082386 | 77016 | 16: 76264493 | 77020 | 16: 76316949 | 77023 | 16: 76382298 |
| 77025 | 16: 76417156 | 77026 | 16: 76424214 | 77029 | 16: 76490659 | 77034 | 16: 76550668 | 77035 | 16: 76561525 |
| 77038 | 16: 76632577 | 77039 | 16: 76645921 | 77044 | 16: 76724147 | 77047 | 16: 76749641 | 77049 | 16: 76810446 |
| 77050 | 16: 76898157 | 77053 | 16: 77026179 | 77066 | 16: 77196110 | 77075 | 16: 77345710 | 77076 | 16: 77345711 |
| 77086 | 16: 77672463 | 77088 | 16: 77700431 | 77133 | 16: 78730933 | 77136 | 16: 78794726 | 77146 | 16: 78936706 |
| 77147 | 16: 78936848 | 77148 | 16: 78936900 | 77162 | 16: 79072375 | 77170 | 16: 79207667 | 77171 | 16: 79214144 |
| 77172 | 16: 79214863 | 77173 | 16: 79214898 | 77174 | 16: 79216805 | 77175 | 16: 79216824 | 77176 | 16: 79216879 |
| 77177 | 16: 79217281 | 77178 | 16: 79246839 | 77179 | 16: 79247022 | 77180 | 16: 79248848 | 77181 | 16: 79252434 |
| 77182 | 16: 79252455 | 77183 | 16: 79253093 | 77184 | 16: 79253212 | 77185 | 16: 79253954 | 77186 | 16: 79256053 |
| 77187 | 16: 79258337 | 77188 | 16: 79258375 | 77189 | 16: 79259607 | 77190 | 16: 79259608 | 77191 | 16: 79259624 |
| 77192 | 16: 79259664 | 77193 | 16: 79259693 | 77194 | 16: 79264907 | 77195 | 16: 79264964 | 77196 | 16: 79265514 |
| 77197 | 16: 79265600 | 77199 | 16: 79316462 | 77203 | 16: 79347181 | 77206 | 16: 79436693 | 77207 | 16: 79449942 |
| 77208 | 16: 79458180 | 77211 | 16: 79513573 | 77212 | 16: 79514562 | 77213 | 16: 79526906 | 77215 | 16: 79554621 |
| 77216 | 16: 79563778 | 77217 | 16: 79626644 | 77219 | 16: 79659075 | 77223 | 16: 79862681 | 77224 | 16: 79881468 |
| 77225 | 16: 79889426 | 77228 | 16: 80019106 | 77231 | 16: 80220495 | 77234 | 16: 80431031 | 77240 | 16: 80869601 |
| 77259 | 17: 425858 | 77280 | 17: 1125457 | 77282 | 17: 1162137 | 77283 | 17: 1162139 | 77284 | 17: 1162184 |
| 77285 | 17: 1162199 | 77286 | 17: 1162200 | 77300 | 17: 1367292 | 77306 | 17: 1539162 | 77316 | 17: 1782217 |
| 77338 | 17: 2449957 | 77357 | 17: 2912361 | 77367 | 17: 3145731 | 77376 | 17: 3382377 | 77391 | 17: 3546676 |
| 77399 | 17: 3801647 | 77403 | 17: 3941038 | 77406 | 17: 3948766 | 77407 | 17: 3948856 | 77408 | 17: 3950488 |
| 77409 | 17: 3950503 | 77410 | 17: 3979701 | 77413 | 17: 4046782 | 77419 | 17: 4192418 | 77424 | 17: 4272755 |
| 77426 | 17: 4298012 | 77432 | 17: 4394112 | 77442 | 17: 4571004 | 77447 | 17: 4633530 | 77448 | 17: 4657147 |
| 77453 | 17: 4721455 | 77454 | 17: 4721515 | 77456 | 17: 4770586 | 77460 | 17: 4835673 | 77465 | 17: 4973219 |
| 77479 | 17: 5317539 | 77481 | 17: 5375833 | 77482 | 17: 5398061 | 77490 | 17: 5679415 | 77491 | 17: 5679423 |
| 77492 | 17: 5679424 | 77493 | 17: 5679464 | 77494 | 17: 5679532 | 77496 | 17: 5728456 | 77512 | 17: 6078923 |
| 77513 | 17: 6107701 | 77516 | 17: 6161274 | 77517 | 17: 6198963 | 77518 | 17: 6205937 | 77520 | 17: 6220260 |
| 77521 | 17: 6229560 | 77522 | 17: 6258944 | 77526 | 17: 6341387 | 77532 | 17: 6459287 | 77533 | 17: 6491029 |
| 77537 | 17: 6552131 | 77539 | 17: 6614404 | 77540 | 17: 6625470 | 77541 | 17: 6626902 | 77544 | 17: 6698968 |
| 77545 | 17: 6701688 | 77546 | 17: 6707615 | 77547 | 17: 6710298 | 77548 | 17: 6715516 | 77554 | 17: 6811535 |
| 77555 | 17: 6819346 | 77559 | 17: 6940926 | 77560 | 17: 6984368 | 77561 | 17: 6985910 | 77563 | 17: 7027194 |
| 77588 | 17: 7507100 | 77596 | 17: 7694458 | 77598 | 17: 7697113 | 77602 | 17: 7770098 | 77612 | 17: 8012088 |
| 77613 | 17: 8012177 | 77621 | 17: 8235574 | 77637 | 17: 8588779 | 77661 | 17: 9138287 | 77674 | 17: 9441496 |
| 77683 | 17: 9768434 | 77684 | 17: 9768476 | 77688 | 17: 9888649 | 77695 | 17: 9978114 | 77696 | 17: 10024882 |
| 77698 | 17: 10050562 | 77700 | 17: 10077655 | 77702 | 17: 10100298 | 77705 | 17: 10110670 | 77709 | 17: 10169816 |
| 77710 | 17: 10176209 | 77717 | 17: 10353846 | 77720 | 17: 10489157 | 77730 | 17: 10813567 | 77731 | 17: 10814317 |
| 77749 | 17: 11330319 | 77765 | 17: 11778925 | 77771 | 17: 11817119 | 77780 | 17: 12090766 | 77797 | 17: 12362004 |
| 77814 | 17: 12766769 | 77823 | 17: 12938879 | 77828 | 17: 13174673 | 77832 | 17: 13361331 | 77837 | 17: 13490837 |
| 77838 | 17: 13490864 | 77849 | 17: 13820980 | 77850 | 17: 13883525 | 77853 | 17: 13941930 | 77865 | 17: 14194666 |
| 77866 | 17: 14208519 | 77867 | 17: 14212322 | 77868 | 17: 14214868 | 77870 | 17: 14226993 | 77871 | 17: 14233961 |
| 77874 | 17: 14255518 | 77875 | 17: 14257411 | 77876 | 17: 14258646 | 77877 | 17: 14265993 | 77878 | 17: 14267278 |
| 77879 | 17: 14268573 | 77885 | 17: 14447098 | 77889 | 17: 14526830 | 77892 | 17: 14548325 | 77897 | 17: 14641198 |
| 77912 | 17: 14905289 | 77913 | 17: 14921802 | 77920 | 17: 15088817 | 77925 | 17: 15159023 | 77929 | 17: 15328788 |
| 77931 | 17: 15328888 | 77937 | 17: 15455174 | 77940 | 17: 15527126 | 77948 | 17: 15652225 | 77952 | 17: 15705665 |
| 77959 | 17: 15911943 | 77967 | 17: 16144638 | 77970 | 17: 16239054 | 77981 | 17: 16560132 | 77983 | 17: 16659063 |
| 77984 | 17: 16684179 | 77989 | 17: 16808806 | 77990 | 17: 16833027 | 78001 | 17: 17127455 | 78002 | 17: 17127599 |
| 78004 | 17: 17132830 | 78007 | 17: 17224074 | 78021 | 17: 17389673 | 78028 | 17: 17475047 | 78029 | 17: 17475831 |
| 78048 | 17: 17963586 | 78050 | 17: 17992691 | 78054 | 17: 18028087 | 78057 | 17: 18062413 | 78060 | 17: 18161738 |
| 78064 | 17: 18284618 | 78087 | 17: 18873882 | 78092 | 17: 18900848 | 78094 | 17: 18961495 | 78102 | 17: 19217997 |
| 78130 | 17: 19581434 | 78135 | 17: 19786181 | 78141 | 17: 19961480 | 78142 | 17: 19965877 | 78144 | 17: 20018582 |
| 78149 | 17: 20111214 | 78152 | 17: 20217310 | 78157 | 17: 20333021 | 78227 | 17: 22641428 | 78231 | 17: 22853179 |
| 78245 | 17: 23369245 | 78266 | 17: 24189137 | 78275 | 17: 24523000 | 78285 | 17: 24792731 | 78294 | 17: 25041571 |
| 78312 | 17: 25581271 | 78323 | 17: 25977654 | 78324 | 17: 25977693 | 78325 | 17: 25977723 | 78326 | 17: 25977762 |
| 78369 | 17: 26883751 | 78370 | 17: 26915514 | 78393 | 17: 27503090 | 78394 | 17: 27559967 | 78399 | 17: 27653769 |
| 78408 | 17: 27921810 | 78413 | 17: 28059892 | 78427 | 17: 28334342 | 78428 | 17: 28334349 | 78439 | 17: 28702389 |
| 78464 | 17: 29480103 | 78469 | 17: 29662529 | 78478 | 17: 29993789 | 78489 | 17: 30232347 | 78490 | 17: 30304435 |
| 78497 | 17: 30487913 | 78510 | 17: 30674204 | 78511 | 17: 30696794 | 78512 | 17: 30709277 | 78516 | 17: 30799626 |
| 78524 | 17: 30996169 | 78544 | 17: 31562261 | 78549 | 17: 31735789 | 78551 | 17: 31735957 | 78561 | 17: 31998920 |
| 78562 | 17: 32030137 | 78571 | 17: 32394529 | 78572 | 17: 32394568 | 78573 | 17: 32403823 | 78574 | 17: 32403842 |
| 78594 | 17: 32777788 | 78599 | 17: 32874912 | 78618 | 17: 33384490 | 78641 | 17: 33842930 | 78655 | 17: 34228544 |
| 78665 | 17: 34544058 | 78678 | 17: 34902455 | 78685 | 17: 35149936 | 78686 | 17: 35186932 | 78694 | 17: 35488740 |
| 78704 | 17: 35746205 | 78706 | 17: 35787842 | 78707 | 17: 35799086 | 78709 | 17: 35857580 | 78712 | 17: 35940925 |
| 78719 | 17: 36144107 | 78728 | 17: 36520470 | 78737 | 17: 36882303 | 78781 | 17: 38220984 | 78810 | 17: 39276967 |
| 78812 | 17: 39326538 | 78837 | 17: 39843444 | 78842 | 17: 39882804 | 78852 | 17: 39935546 | 78860 | 17: 40113609 |
| 78865 | 17: 40279234 | 78867 | 17: 40310128 | 78868 | 17: 40314686 | 78879 | 17: 40566975 | 78883 | 17: 40575300 |
| 78886 | 17: 40597293 | 78894 | 17: 40765558 | 78897 | 17: 40880376 | 78898 | 17: 40882503 | 78905 | 17: 41002973 |
| 78932 | 17: 41864826 | 78941 | 17: 42170894 | 78942 | 17: 42186823 | 78943 | 17: 42188310 | 78946 | 17: 42229273 |
| 78954 | 17: 42393090 | 78956 | 17: 42415227 | 78970 | 17: 42715046 | 78976 | 17: 42768963 | 78978 | 17: 42821182 |
| 78982 | 17: 42891704 | 78983 | 17: 42923614 | 78997 | 17: 43357874 | 79000 | 17: 43436343 | 79003 | 17: 43553874 |
| 79006 | 17: 43577102 | 79007 | 17: 43577588 | 79008 | 17: 43594706 | 79011 | 17: 43609022 | 79012 | 17: 43623722 |
| 79029 | 17: 44194890 | 79032 | 17: 44224761 | 79040 | 17: 44531668 | 79046 | 17: 44677660 | 79052 | 17: 44819760 |
| 79058 | 17: 44927880 | 79063 | 17: 45086949 | 79064 | 17: 45091135 | 79066 | 17: 45172949 | 79068 | 17: 45178502 |
| 79069 | 17: 45178540 | 79070 | 17: 45180519 | 79071 | 17: 45180612 | 79072 | 17: 45181370 | 79073 | 17: 45182722 |
| 79091 | 17: 45616057 | 79094 | 17: 45734727 | 79112 | 17: 46168639 | 79113 | 17: 46171496 | 79127 | 17: 46408391 |
| 79135 | 17: 46615898 | 79136 | 17: 46624459 | 79142 | 17: 46676910 | 79187 | 17: 47856534 | 79215 | 17: 48637182 |
| 79244 | 17: 49321412 | 79268 | 17: 49848060 | 79273 | 17: 49923094 | 79285 | 17: 50262075 | 79287 | 17: 50270777 |
| 79288 | 17: 50274476 | 79315 | 17: 50731825 | 79316 | 17: 50769132 | 79319 | 17: 50841533 | 79320 | 17: 50864647 |
| 79322 | 17: 50902320 | 79326 | 17: 50955494 | 79327 | 17: 50959261 | 79339 | 17: 51345327 | 79342 | 17: 51469617 |
| 79343 | 17: 51470914 | 79346 | 17: 51529156 | 79348 | 17: 51546917 | 79355 | 17: 51627470 | 79359 | 17: 51685973 |
| 79360 | 17: 51689778 | 79364 | 17: 51762211 | 79366 | 17: 51792442 | 79368 | 17: 51813934 | 79375 | 17: 51958814 |
| 79377 | 17: 51959782 | 79379 | 17: 52027381 | 79395 | 17: 52307367 | 79403 | 17: 52573801 | 79414 | 17: 52988658 |
| 79417 | 17: 53020835 | 79423 | 17: 53275351 | 79428 | 17: 53388401 | 79430 | 17: 53390888 | 79431 | 17: 53448341 |
| 79433 | 17: 53470903 | 79435 | 17: 53532750 | 79447 | 17: 53919113 | 79454 | 17: 54025669 | 79455 | 17: 54029457 |
| 79456 | 17: 54030902 | 79457 | 17: 54033380 | 79458 | 17: 54062683 | 79459 | 17: 54122851 | 79461 | 17: 54128175 |
| 79465 | 17: 54163192 | 79468 | 17: 54264980 | 79469 | 17: 54267012 | 79470 | 17: 54310765 | 79483 | 17: 54668554 |
| 79489 | 17: 54807887 | 79499 | 17: 54895464 | 79505 | 17: 55074569 | 79506 | 17: 55081286 | 79507 | 17: 55082228 |
| 79508 | 17: 55105646 | 79509 | 17: 55105686 | 79512 | 17: 55385614 | 79517 | 17: 55484565 | 79521 | 17: 55592718 |
| 79523 | 17: 55665541 | 79524 | 17: 55716549 | 79526 | 17: 55752066 | 79532 | 17: 55876873 | 79537 | 17: 56011560 |
| 79548 | 17: 56279452 | 79549 | 17: 56281095 | 79560 | 17: 56566250 | 79564 | 17: 56651078 | 79565 | 17: 56710101 |
| 79571 | 17: 56976866 | 79576 | 17: 57116303 | 79583 | 17: 57285859 | 79591 | 17: 57472146 | 79598 | 17: 57643506 |
| 79601 | 17: 57716356 | 79602 | 17: 57730355 | 79603 | 17: 57731200 | 79610 | 17: 57797646 | 79612 | 17: 57831569 |
| 79623 | 17: 58093779 | 79625 | 17: 58112817 | 79626 | 17: 58130419 | 79627 | 17: 58141075 | 79634 | 17: 58304066 |
| 79665 | 17: 58556801 | 79670 | 17: 58775371 | 79679 | 17: 59008052 | 79688 | 17: 59157020 | 79690 | 17: 59215735 |
| 79700 | 17: 59384904 | 79702 | 17: 59397574 | 79710 | 17: 59604994 | 79714 | 17: 59716548 | 79719 | 17: 59839482 |
| 79730 | 17: 60067619 | 79731 | 17: 60090877 | 79732 | 17: 60090928 | 79733 | 17: 60097662 | 79743 | 17: 60248908 |
| 79751 | 17: 60373041 | 79752 | 17: 60374859 | 79753 | 17: 60395890 | 79754 | 17: 60395994 | 79760 | 17: 60500390 |
| 79771 | 17: 60710033 | 79772 | 17: 60710035 | 79773 | 17: 60710040 | 79774 | 17: 60710101 | 79779 | 17: 60795229 |
| 79780 | 17: 60795274 | 79781 | 17: 60795346 | 79784 | 17: 60843399 | 79807 | 17: 61141160 | 79812 | 17: 61202979 |
| 79814 | 17: 61208274 | 79815 | 17: 61208429 | 79816 | 17: 61237780 | 79819 | 17: 61316287 | 79822 | 17: 61370873 |
| 79828 | 17: 61499579 | 79829 | 17: 61499616 | 79842 | 17: 61892683 | 79846 | 17: 61971343 | 79850 | 17: 62029219 |
| 79858 | 17: 62310603 | 79866 | 17: 62556427 | 79874 | 17: 62749389 | 79882 | 17: 62937635 | 79884 | 17: 62967008 |
| 79886 | 17: 63060456 | 79890 | 17: 63193363 | 79904 | 17: 63343949 | 79909 | 17: 63512971 | 79912 | 17: 63562509 |
| 79916 | 17: 63612425 | 79917 | 17: 63613044 | 79918 | 17: 63614883 | 79919 | 17: 63615321 | 79920 | 17: 63615338 |
| 79921 | 17: 63619368 | 79922 | 17: 63619438 | 79923 | 17: 63619955 | 79926 | 17: 63623292 | 79927 | 17: 63624426 |
| 79933 | 17: 63696661 | 79937 | 17: 63745078 | 79938 | 17: 63765593 | 79946 | 17: 63909068 | 79947 | 17: 63911823 |
| 79948 | 17: 63914506 | 79949 | 17: 63924591 | 79953 | 17: 64073618 | 79954 | 17: 64077337 | 79955 | 17: 64079380 |
| 79957 | 17: 64164363 | 79959 | 17: 64233930 | 79963 | 17: 64320209 | 79964 | 17: 64364133 | 79965 | 17: 64376618 |
| 79966 | 17: 64381714 | 79967 | 17: 64390370 | 79968 | 17: 64391421 | 79969 | 17: 64392375 | 79971 | 17: 64400070 |
| 79975 | 17: 64543414 | 79983 | 17: 64868669 | 79986 | 17: 64936914 | 80004 | 17: 65250897 | 80005 | 17: 65252978 |
| 80007 | 17: 65269001 | 80009 | 17: 65311178 | 80015 | 17: 65495113 | 80016 | 17: 65515359 | 80028 | 17: 65711335 |
| 80046 | 17: 66084982 | 80048 | 17: 66098132 | 80091 | 17: 66926217 | 80093 | 17: 66962321 | 80094 | 17: 66963375 |
| 80095 | 17: 66964444 | 80096 | 17: 66980228 | 80097 | 17: 66981196 | 80098 | 17: 66983892 | 80099 | 17: 66987390 |
| 80100 | 17: 66989033 | 80101 | 17: 66991682 | 80102 | 17: 66993583 | 80117 | 17: 67452220 | 80120 | 17: 67493854 |
| 80123 | 17: 67576683 | 80133 | 17: 67991825 | 80143 | 17: 68190606 | 80145 | 17: 68235847 | 80146 | 17: 68237141 |
| 80152 | 17: 68336966 | 80154 | 17: 68390649 | 80156 | 17: 68404948 | 80165 | 17: 68610408 | 80169 | 17: 68711761 |
| 80177 | 17: 68924124 | 80186 | 17: 69025532 | 80187 | 17: 69025781 | 80191 | 17: 69104813 | 80200 | 17: 69235524 |
| 80201 | 17: 69244183 | 80208 | 17: 69526999 | 80209 | 17: 69557728 | 80210 | 17: 69558386 | 80221 | 17: 69723039 |
| 80226 | 17: 69823676 | 80236 | 17: 70089184 | 80242 | 17: 70183712 | 80266 | 17: 70611985 | 80267 | 17: 70615923 |
| 80268 | 17: 70617839 | 80269 | 17: 70618022 | 80270 | 17: 70618023 | 80271 | 17: 70619770 | 80272 | 17: 70621442 |
| 80294 | 17: 71216081 | 80295 | 17: 71224018 | 80297 | 17: 71275992 | 80298 | 17: 71276089 | 80299 | 17: 71276886 |
| 80300 | 17: 71276966 | 80301 | 17: 71283804 | 80306 | 17: 71441658 | 80309 | 17: 71512874 | 80310 | 17: 71518391 |
| 80311 | 17: 71528096 | 80312 | 17: 71528119 | 80313 | 17: 71528120 | 80314 | 17: 71528861 | 80315 | 17: 71528882 |
| 80316 | 17: 71528942 | 80324 | 17: 71695918 | 80325 | 17: 71702829 | 80328 | 17: 71812939 | 80339 | 17: 71951302 |
| 80349 | 17: 72204450 | 80356 | 17: 72342109 | 80358 | 17: 72363855 | 80360 | 17: 72369402 | 80362 | 17: 72398155 |
| 80364 | 17: 72408383 | 80365 | 17: 72445427 | 80366 | 17: 72447709 | 80367 | 17: 72457694 | 80369 | 17: 72468245 |
| 80370 | 17: 72498960 | 80374 | 17: 72555058 | 80384 | 17: 72883784 | 80386 | 17: 72916533 | 80391 | 17: 73036168 |
| 80411 | 18: 868580 | 80413 | 18: 911112 | 80414 | 18: 911196 | 80415 | 18: 932024 | 80416 | 18: 932036 |
| 80417 | 18: 932132 | 80431 | 18: 1178922 | 80435 | 18: 1215919 | 80436 | 18: 1269485 | 80437 | 18: 1269511 |
| 80438 | 18: 1269570 | 80452 | 18: 1553979 | 80453 | 18: 1565391 | 80460 | 18: 1658820 | 80467 | 18: 1733905 |
| 80468 | 18: 1734779 | 80469 | 18: 1737507 | 80470 | 18: 1739009 | 80471 | 18: 1741139 | 80472 | 18: 1742546 |
| 80474 | 18: 1743718 | 80475 | 18: 1744700 | 80476 | 18: 1745880 | 80477 | 18: 1746570 | 80478 | 18: 1747824 |
| 80479 | 18: 1749671 | 80480 | 18: 1751021 | 80481 | 18: 1752037 | 80482 | 18: 1753160 | 80483 | 18: 1754297 |
| 80484 | 18: 1754989 | 80485 | 18: 1755466 | 80486 | 18: 1758653 | 80487 | 18: 1759177 | 80488 | 18: 1759407 |
| 80489 | 18: 1759775 | 80490 | 18: 1760828 | 80491 | 18: 1761351 | 80492 | 18: 1761856 | 80493 | 18: 1764061 |
| 80494 | 18: 1765063 | 80495 | 18: 1765811 | 80496 | 18: 1766413 | 80497 | 18: 1766979 | 80498 | 18: 1780698 |
| 80499 | 18: 1799373 | 80503 | 18: 1821333 | 80515 | 18: 2132264 | 80519 | 18: 2189010 | 80525 | 18: 2310539 |
| 80529 | 18: 2407203 | 80532 | 18: 2547395 | 80535 | 18: 2627721 | 80548 | 18: 2801078 | 80549 | 18: 2824948 |
| 80553 | 18: 2893572 | 80559 | 18: 2948533 | 80590 | 18: 3732736 | 80593 | 18: 3803407 | 80599 | 18: 3918400 |
| 80604 | 18: 4117718 | 80610 | 18: 4175893 | 80622 | 18: 4456924 | 80623 | 18: 4458325 | 80652 | 18: 5105107 |
| 80657 | 18: 5261361 | 80658 | 18: 5303405 | 80667 | 18: 5381034 | 80668 | 18: 5392289 | 80686 | 18: 5796418 |
| 80695 | 18: 6075338 | 80699 | 18: 6140925 | 80700 | 18: 6150654 | 80702 | 18: 6155426 | 80708 | 18: 6179950 |
| 80710 | 18: 6195750 | 80717 | 18: 6307757 | 80732 | 18: 6411938 | 80733 | 18: 6414217 | 80734 | 18: 6434042 |
| 80736 | 18: 6473072 | 80742 | 18: 6606486 | 80756 | 18: 6904961 | 80759 | 18: 6952523 | 80775 | 18: 7393588 |
| 80782 | 18: 7571670 | 80783 | 18: 7571744 | 80784 | 18: 7571766 | 80790 | 18: 7722433 | 80791 | 18: 7725494 |
| 80792 | 18: 7759211 | 80794 | 18: 7785017 | 80795 | 18: 7806089 | 80798 | 18: 7857634 | 80809 | 18: 8151586 |
| 80814 | 18: 8174600 | 80816 | 18: 8217290 | 80817 | 18: 8219384 | 80818 | 18: 8219385 | 80819 | 18: 8219497 |
| 80836 | 18: 8556730 | 80847 | 18: 8895607 | 80850 | 18: 8930714 | 80868 | 18: 9094422 | 80869 | 18: 9121273 |
| 80871 | 18: 9155492 | 80873 | 18: 9160707 | 80877 | 18: 9224812 | 80879 | 18: 9281528 | 80891 | 18: 9500208 |
| 80893 | 18: 9514100 | 80897 | 18: 9541606 | 80898 | 18: 9560416 | 80903 | 18: 9622228 | 80913 | 18: 9939597 |
| 80915 | 18: 9939685 | 80920 | 18: 10053603 | 80937 | 18: 10466956 | 80938 | 18: 10467016 | 80939 | 18: 10474781 |
| 80940 | 18: 10474891 | 80944 | 18: 10554759 | 80945 | 18: 10555268 | 80949 | 18: 10623982 | 80954 | 18: 10638844 |
| 80955 | 18: 10661516 | 80959 | 18: 10775715 | 80966 | 18: 10924943 | 80970 | 18: 11032803 | 80972 | 18: 11076163 |
| 80975 | 18: 11143812 | 80978 | 18: 11177661 | 80979 | 18: 11189421 | 80982 | 18: 11197228 | 80985 | 18: 11315060 |
| 80986 | 18: 11322791 | 80987 | 18: 11333644 | 80988 | 18: 11340350 | 80989 | 18: 11349111 | 80990 | 18: 11352571 |
| 80991 | 18: 11362283 | 80992 | 18: 11367578 | 80993 | 18: 11398671 | 80995 | 18: 11499305 | 80996 | 18: 11499360 |
| 80997 | 18: 11589376 | 80999 | 18: 11631258 | 81001 | 18: 11639177 | 81002 | 18: 11640852 | 81004 | 18: 11652518 |
| 81007 | 18: 11719208 | 81026 | 18: 12374145 | 81030 | 18: 12487243 | 81036 | 18: 12677943 | 81058 | 18: 13316111 |
| 81059 | 18: 13322081 | 81060 | 18: 13326301 | 81068 | 18: 13483552 | 81090 | 18: 14035593 | 81092 | 18: 14117250 |
| 81097 | 18: 14266021 | 81104 | 18: 14579399 | 81110 | 18: 14880493 | 81111 | 18: 14918649 | 81121 | 18: 15157858 |
| 81129 | 18: 15352349 | 81130 | 18: 15353723 | 81136 | 18: 15529204 | 81137 | 18: 15532676 | 81138 | 18: 15535125 |
| 81139 | 18: 15537825 | 81142 | 18: 15574419 | 81145 | 18: 15607698 | 81147 | 18: 15643821 | 81149 | 18: 15771762 |
| 81163 | 18: 16246520 | 81164 | 18: 16246606 | 81165 | 18: 16246649 | 81167 | 18: 16294087 | 81170 | 18: 16339724 |
| 81179 | 18: 16491063 | 81186 | 18: 16701247 | 81188 | 18: 16722963 | 81191 | 18: 16855403 | 81194 | 18: 16929518 |
| 81198 | 18: 16960682 | 81205 | 18: 17095383 | 81206 | 18: 17095391 | 81207 | 18: 17095421 | 81208 | 18: 17128188 |
| 81214 | 18: 17231296 | 81219 | 18: 17332696 | 81224 | 18: 17474883 | 81225 | 18: 17474962 | 81246 | 18: 17964994 |
| 81248 | 18: 18015282 | 81259 | 18: 18234827 | 81274 | 18: 18340451 | 81277 | 18: 18455809 | 81281 | 18: 18555382 |
| 81286 | 18: 18634395 | 81288 | 18: 18652008 | 81292 | 18: 18693621 | 81296 | 18: 18741396 | 81297 | 18: 18766595 |
| 81304 | 18: 18991234 | 81308 | 18: 19028089 | 81312 | 18: 19104084 | 81318 | 18: 19279732 | 81319 | 18: 19306933 |
| 81321 | 18: 19321180 | 81328 | 18: 19549103 | 81337 | 18: 19828522 | 81341 | 18: 19884211 | 81346 | 18: 19992495 |
| 81379 | 18: 20783780 | 81396 | 18: 21231861 | 81397 | 18: 21231877 | 81404 | 18: 21429864 | 81405 | 18: 21429866 |
| 81419 | 18: 21805702 | 81422 | 18: 21842921 | 81427 | 18: 21915772 | 81428 | 18: 21944535 | 81429 | 18: 21944650 |
| 81439 | 18: 22075634 | 81459 | 18: 22432351 | 81467 | 18: 22574217 | 81468 | 18: 22581424 | 81470 | 18: 22616518 |
| 81489 | 18: 23048862 | 81515 | 18: 23639968 | 81523 | 18: 23885882 | 81526 | 18: 23978279 | 81530 | 18: 24024754 |
| 81531 | 18: 24028549 | 81532 | 18: 24039248 | 81538 | 18: 24159516 | 81541 | 18: 24233256 | 81547 | 18: 24449226 |
| 81548 | 18: 24467752 | 81551 | 18: 24569333 | 81555 | 18: 24603788 | 81556 | 18: 24605526 | 81557 | 18: 24606504 |
| 81558 | 18: 24609637 | 81559 | 18: 24622318 | 81560 | 18: 24626076 | 81561 | 18: 24627052 | 81562 | 18: 24640038 |
| 81563 | 18: 24646900 | 81564 | 18: 24648311 | 81565 | 18: 24650296 | 81566 | 18: 24653596 | 81567 | 18: 24655887 |
| 81568 | 18: 24658898 | 81569 | 18: 24680546 | 81572 | 18: 24703105 | 81573 | 18: 24711358 | 81581 | 18: 24916840 |
| 81586 | 18: 25087427 | 81594 | 18: 25243259 | 81595 | 18: 25263618 | 81597 | 18: 25320467 | 81603 | 18: 25475882 |
| 81604 | 18: 25507140 | 81608 | 18: 25560734 | 81613 | 18: 25671777 | 81614 | 18: 25693559 | 81617 | 18: 25769838 |
| 81618 | 18: 25772348 | 81619 | 18: 25777563 | 81622 | 18: 25833720 | 81626 | 18: 25944954 | 81638 | 18: 26118698 |
| 81643 | 18: 26252156 | 81648 | 18: 26367037 | 81652 | 18: 26448739 | 81674 | 18: 26911688 | 81683 | 18: 27123603 |
| 81711 | 18: 27938774 | 81712 | 18: 27938830 | 81733 | 18: 28523029 | 81752 | 18: 29298380 | 81753 | 18: 29324758 |
| 81755 | 18: 29354497 | 81756 | 18: 29354560 | 81757 | 18: 29354692 | 81758 | 18: 29356162 | 81759 | 18: 29359726 |
| 81760 | 18: 29359801 | 81765 | 18: 29507594 | 81766 | 18: 29507596 | 81767 | 18: 29507695 | 81768 | 18: 29517248 |
| 81800 | 18: 30444281 | 81812 | 18: 30824413 | 81820 | 18: 31114666 | 81821 | 18: 31161073 | 81829 | 18: 31403926 |
| 81860 | 18: 32193066 | 81865 | 18: 32277972 | 81866 | 18: 32277986 | 81889 | 18: 32769354 | 81892 | 18: 32797023 |
| 81893 | 18: 32809799 | 81909 | 18: 33174459 | 81913 | 18: 33266043 | 81915 | 18: 33332355 | 81928 | 18: 33524126 |
| 81938 | 18: 33726109 | 81942 | 18: 33803131 | 81943 | 18: 33819518 | 81948 | 18: 33891121 | 81950 | 18: 33985539 |
| 81958 | 18: 34273373 | 81960 | 18: 34305936 | 81962 | 18: 34318674 | 81963 | 18: 34340817 | 81967 | 18: 34415049 |
| 81968 | 18: 34460275 | 81969 | 18: 34460342 | 81971 | 18: 34477636 | 81973 | 18: 34507746 | 81976 | 18: 34573297 |
| 81979 | 18: 34674118 | 81981 | 18: 34742637 | 81985 | 18: 34868221 | 81993 | 18: 35213610 | 81994 | 18: 35247365 |
| 81996 | 18: 35258480 | 82001 | 18: 35371180 | 82003 | 18: 35406541 | 82008 | 18: 35505165 | 82009 | 18: 35515593 |
| 82010 | 18: 35575292 | 82012 | 18: 35602009 | 82016 | 18: 35713513 | 82021 | 18: 35869059 | 82027 | 18: 35949415 |
| 82029 | 18: 35961563 | 82035 | 18: 36058481 | 82036 | 18: 36063927 | 82038 | 18: 36087372 | 82039 | 18: 36087440 |
| 82040 | 18: 36093659 | 82042 | 18: 36121381 | 82046 | 18: 36318653 | 82054 | 18: 36585013 | 82059 | 18: 36712932 |
| 82066 | 18: 36883800 | 82067 | 18: 36890174 | 82069 | 18: 36892550 | 82093 | 18: 37455502 | 82098 | 18: 37533228 |
| 82102 | 18: 37619375 | 82122 | 18: 38001454 | 82131 | 18: 38196771 | 82133 | 18: 38218416 | 82135 | 18: 38277631 |
| 82138 | 18: 38301449 | 82141 | 18: 38331320 | 82150 | 18: 38614192 | 82174 | 18: 39233646 | 82175 | 18: 39286312 |
| 82180 | 18: 39414004 | 82182 | 18: 39447432 | 82189 | 18: 39589574 | 82192 | 18: 39612914 | 82215 | 18: 40137391 |
| 82216 | 18: 40137453 | 82217 | 18: 40138746 | 82218 | 18: 40138750 | 82219 | 18: 40138985 | 82220 | 18: 40139048 |
| 82222 | 18: 40176507 | 82223 | 18: 40176673 | 82228 | 18: 40316884 | 82230 | 18: 40330132 | 82237 | 18: 40533358 |
| 82238 | 18: 40534569 | 82241 | 18: 40597479 | 82249 | 18: 40763764 | 82250 | 18: 40763815 | 82254 | 18: 40819026 |
| 82255 | 18: 40819036 | 82261 | 18: 40956148 | 82269 | 18: 41180952 | 82274 | 18: 41372533 | 82275 | 18: 41391080 |
| 82277 | 18: 41412597 | 82304 | 18: 42002651 | 82308 | 18: 42064307 | 82314 | 18: 42154655 | 82325 | 18: 42312455 |
| 82342 | 18: 42673603 | 82352 | 18: 42839761 | 82355 | 18: 42870031 | 82356 | 18: 42870306 | 82357 | 18: 42870636 |
| 82373 | 18: 43209321 | 82378 | 18: 43300251 | 82397 | 18: 43597221 | 82399 | 18: 43723768 | 82400 | 18: 43729208 |
| 82401 | 18: 43744525 | 82403 | 18: 43765343 | 82415 | 18: 44100679 | 82418 | 18: 44219776 | 82433 | 18: 44678130 |
| 82437 | 18: 44746464 | 82440 | 18: 44842043 | 82444 | 18: 44999448 | 82448 | 18: 45230454 | 82451 | 18: 45235853 |
| 82453 | 18: 45282086 | 82454 | 18: 45368501 | 82457 | 18: 45512461 | 82459 | 18: 45555708 | 82460 | 18: 45555738 |
| 82461 | 18: 45555791 | 82463 | 18: 45568910 | 82474 | 18: 45823041 | 82477 | 18: 45919734 | 82481 | 18: 46058902 |
| 82482 | 18: 46058908 | 82483 | 18: 46059262 | 82484 | 18: 46059448 | 82485 | 18: 46059625 | 82486 | 18: 46074456 |
| 82488 | 18: 46098110 | 82491 | 18: 46151119 | 82493 | 18: 46204752 | 82500 | 18: 46339447 | 82506 | 18: 46411478 |
| 82507 | 18: 46412270 | 82510 | 18: 46452569 | 82511 | 18: 46454259 | 82514 | 18: 46462113 | 82515 | 18: 46472984 |
| 82516 | 18: 46474223 | 82517 | 18: 46474231 | 82518 | 18: 46474239 | 82519 | 18: 46474366 | 82520 | 18: 46480475 |
| 82521 | 18: 46481003 | 82522 | 18: 46481046 | 82523 | 18: 46481053 | 82524 | 18: 46481480 | 82525 | 18: 46481499 |
| 82526 | 18: 46489662 | 82527 | 18: 46490303 | 82528 | 18: 46490881 | 82529 | 18: 46498795 | 82530 | 18: 46499716 |
| 82531 | 18: 46500155 | 82532 | 18: 46511353 | 82533 | 18: 46512103 | 82534 | 18: 46512104 | 82535 | 18: 46523855 |
| 82536 | 18: 46528787 | 82540 | 18: 46627346 | 82542 | 18: 46696595 | 82543 | 18: 46697798 | 82544 | 18: 46727043 |
| 82546 | 18: 46727075 | 82547 | 18: 46731490 | 82548 | 18: 46731757 | 82549 | 18: 46731785 | 82550 | 18: 46731861 |
| 82551 | 18: 46742887 | 82552 | 18: 46742932 | 82553 | 18: 46742940 | 82554 | 18: 46743003 | 82555 | 18: 46745292 |
| 82556 | 18: 46745318 | 82557 | 18: 46745397 | 82558 | 18: 46745402 | 82559 | 18: 46747438 | 82560 | 18: 46747523 |
| 82561 | 18: 46751198 | 82562 | 18: 46751982 | 82563 | 18: 46751987 | 82564 | 18: 46752090 | 82565 | 18: 46754516 |
| 82567 | 18: 46762245 | 82568 | 18: 46762248 | 82569 | 18: 46763772 | 82570 | 18: 46764309 | 82571 | 18: 46764530 |
| 82572 | 18: 46764820 | 82573 | 18: 46764937 | 82574 | 18: 46765974 | 82575 | 18: 46766007 | 82576 | 18: 46766010 |
| 82577 | 18: 46771417 | 82578 | 18: 46772451 | 82581 | 18: 46793532 | 82585 | 18: 46926291 | 82586 | 18: 46926325 |
| 82587 | 18: 46935389 | 82588 | 18: 46936632 | 82589 | 18: 46936776 | 82592 | 18: 46961882 | 82593 | 18: 46987640 |
| 82606 | 18: 47331147 | 82607 | 18: 47350791 | 82609 | 18: 47380300 | 82614 | 18: 47561882 | 82615 | 18: 47569792 |
| 82616 | 18: 47584273 | 82623 | 18: 47724055 | 82630 | 18: 47880052 | 82632 | 18: 47904454 | 82633 | 18: 47905402 |
| 82635 | 18: 47914575 | 82638 | 18: 47982264 | 82639 | 18: 47986634 | 82645 | 18: 48080577 | 82646 | 18: 48082036 |
| 82647 | 18: 48092307 | 82648 | 18: 48093267 | 82649 | 18: 48094554 | 82650 | 18: 48095808 | 82651 | 18: 48097168 |
| 82652 | 18: 48098873 | 82653 | 18: 48099751 | 82654 | 18: 48100359 | 82655 | 18: 48102403 | 82656 | 18: 48103383 |
| 82657 | 18: 48105090 | 82658 | 18: 48105806 | 82660 | 18: 48115016 | 82661 | 18: 48119818 | 82662 | 18: 48164016 |
| 82669 | 18: 48328458 | 82674 | 18: 48486626 | 82677 | 18: 48540681 | 82678 | 18: 48540711 | 82681 | 18: 48550284 |
| 82682 | 18: 48552506 | 82684 | 18: 48648276 | 82685 | 18: 48661989 | 82692 | 18: 48824317 | 82700 | 18: 49125312 |
| 82709 | 18: 49431844 | 82710 | 18: 49440743 | 82718 | 18: 49685722 | 82720 | 18: 49690710 | 82722 | 18: 49774209 |
| 82727 | 18: 49905675 | 82734 | 18: 49974216 | 82744 | 18: 50307933 | 82768 | 18: 50909576 | 82776 | 18: 51192874 |
| 82780 | 18: 51273762 | 82784 | 18: 51393314 | 82799 | 18: 51647623 | 82801 | 18: 51684659 | 82803 | 18: 51687197 |
| 82804 | 18: 51691345 | 82805 | 18: 51691402 | 82806 | 18: 51691426 | 82807 | 18: 51691907 | 82808 | 18: 51692088 |
| 82810 | 18: 51703006 | 82811 | 18: 51703434 | 82813 | 18: 51710422 | 82820 | 18: 51868158 | 82821 | 18: 51868247 |
| 82822 | 18: 51877833 | 82823 | 18: 51888735 | 82826 | 18: 51959716 | 82833 | 18: 52170747 | 82843 | 18: 52408540 |
| 82844 | 18: 52434788 | 82846 | 18: 52463921 | 82848 | 18: 52526615 | 82849 | 18: 52569137 | 82850 | 18: 52586631 |
| 82856 | 18: 52750690 | 82863 | 18: 53056273 | 82864 | 18: 53066684 | 82876 | 18: 53447947 | 82877 | 18: 53462716 |
| 82878 | 18: 53515653 | 82880 | 18: 53545259 | 82883 | 18: 53565634 | 82885 | 18: 53596284 | 82889 | 18: 53631016 |
| 82890 | 18: 53635483 | 82891 | 18: 53666625 | 82892 | 18: 53680050 | 82902 | 18: 53873089 | 82903 | 18: 53882150 |
| 82907 | 18: 53924797 | 82908 | 18: 53926298 | 82909 | 18: 53938427 | 82910 | 18: 53965820 | 82911 | 18: 53970987 |
| 82912 | 18: 53987026 | 82913 | 18: 53988178 | 82917 | 18: 54051234 | 82922 | 18: 54129853 | 82924 | 18: 54256059 |
| 82929 | 18: 54326329 | 82930 | 18: 54348038 | 82931 | 18: 54348158 | 82932 | 18: 54356059 | 82943 | 18: 54594314 |
| 82945 | 18: 54606774 | 82946 | 18: 54607245 | 82947 | 18: 54615284 | 82948 | 18: 54615550 | 82949 | 18: 54618878 |
| 82951 | 18: 54632037 | 82952 | 18: 54674333 | 82956 | 18: 54725999 | 82957 | 18: 54726122 | 82958 | 18: 54732855 |
| 82965 | 18: 54920625 | 82978 | 18: 55105119 | 82980 | 18: 55125421 | 82981 | 18: 55130089 | 82991 | 18: 55397342 |
| 82993 | 18: 55448102 | 82994 | 18: 55479044 | 83003 | 18: 55722088 | 83006 | 18: 55855289 | 83010 | 18: 55908909 |
| 83022 | 18: 56235646 | 83025 | 18: 56256914 | 83027 | 18: 56286701 | 83028 | 18: 56286760 | 83030 | 18: 56311848 |
| 83037 | 18: 56458961 | 83039 | 18: 56480496 | 83040 | 18: 56481635 | 83041 | 18: 56500189 | 83043 | 18: 56516815 |
| 83046 | 18: 56550331 | 83051 | 18: 56657542 | 83053 | 18: 56669005 | 83054 | 18: 56669128 | 83055 | 18: 56679475 |
| 83114 | 18: 57068537 | 83115 | 18: 57072585 | 83119 | 18: 57151314 | 83120 | 18: 57195331 | 83126 | 18: 57435685 |
| 83134 | 18: 57498462 | 83140 | 18: 57652070 | 83141 | 18: 57658393 | 83142 | 18: 57662151 | 83143 | 18: 57673429 |
| 83149 | 18: 57750585 | 83150 | 18: 57764229 | 83151 | 18: 57774781 | 83159 | 18: 57875057 | 83160 | 18: 57880006 |
| 83162 | 18: 57922196 | 83163 | 18: 57922208 | 83168 | 18: 57950000 | 83184 | 18: 58213698 | 83185 | 18: 58215105 |
| 83186 | 18: 58221738 | 83187 | 18: 58237470 | 83189 | 18: 58270823 | 83190 | 18: 58280185 | 83191 | 18: 58285064 |
| 83192 | 18: 58286528 | 83193 | 18: 58288374 | 83194 | 18: 58289386 | 83195 | 18: 58290105 | 83196 | 18: 58292173 |
| 83197 | 18: 58292702 | 83198 | 18: 58293461 | 83199 | 18: 58294515 | 83200 | 18: 58296803 | 83201 | 18: 58297833 |
| 83202 | 18: 58298676 | 83203 | 18: 58302578 | 83204 | 18: 58303580 | 83205 | 18: 58304589 | 83206 | 18: 58306121 |
| 83207 | 18: 58308817 | 83208 | 18: 58309408 | 83209 | 18: 58311748 | 83210 | 18: 58314758 | 83211 | 18: 58319283 |
| 83213 | 18: 58328404 | 83214 | 18: 58335688 | 83215 | 18: 58336548 | 83217 | 18: 58339398 | 83218 | 18: 58340297 |
| 83219 | 18: 58341452 | 83220 | 18: 58343184 | 83221 | 18: 58346217 | 83222 | 18: 58359203 | 83223 | 18: 58360688 |
| 83224 | 18: 58362462 | 83225 | 18: 58369410 | 83226 | 18: 58372976 | 83228 | 18: 58501449 | 83259 | 18: 59941034 |
| 83264 | 18: 60057741 | 83276 | 18: 60557876 | 83284 | 18: 60892284 | 83285 | 18: 60892325 | 83286 | 18: 60892345 |
| 83287 | 18: 60908498 | 83296 | 18: 61230100 | 83297 | 18: 61248798 | 83306 | 18: 61528542 | 83314 | 18: 61669548 |
| 83316 | 18: 61688550 | 83327 | 18: 61924215 | 83328 | 18: 61925438 | 83329 | 18: 61925595 | 83331 | 18: 61938092 |
| 83332 | 18: 61941006 | 83333 | 18: 61948334 | 83334 | 18: 61949940 | 83336 | 18: 61962954 | 83337 | 18: 61968568 |
| 83338 | 18: 61969417 | 83339 | 18: 61975282 | 83340 | 18: 61976235 | 83341 | 18: 61977792 | 83343 | 18: 61979272 |
| 83344 | 18: 61986664 | 83345 | 18: 61987641 | 83347 | 18: 62017097 | 83348 | 18: 62031220 | 83350 | 18: 62050366 |
| 83351 | 18: 62055500 | 83352 | 18: 62069703 | 83355 | 18: 62098858 | 83356 | 18: 62119696 | 83365 | 18: 62301361 |
| 83369 | 18: 62396313 | 83370 | 18: 62411556 | 83371 | 18: 62412333 | 83372 | 18: 62491921 | 83391 | 18: 63189938 |
| 83392 | 18: 63193118 | 83393 | 18: 63203763 | 83396 | 18: 63217724 | 83397 | 18: 63217760 | 83398 | 18: 63217761 |
| 83401 | 18: 63282873 | 83406 | 18: 63442841 | 83407 | 18: 63443616 | 83410 | 18: 63449366 | 83413 | 18: 63478461 |
| 83420 | 18: 63566906 | 83435 | 18: 63851165 | 83438 | 18: 63870217 | 83439 | 18: 63870560 | 83440 | 18: 63870634 |
| 83441 | 18: 63878287 | 83446 | 18: 64020285 | 83448 | 18: 64103695 | 83449 | 18: 64107247 | 83450 | 18: 64118087 |
| 83459 | 18: 64261304 | 83464 | 18: 64351790 | 83473 | 18: 64453963 | 83475 | 18: 64457710 | 83480 | 18: 64586875 |
| 83481 | 18: 64586957 | 83483 | 18: 64587029 | 83484 | 18: 64588207 | 83485 | 18: 64588277 | 83486 | 18: 64594228 |
| 83489 | 18: 64607295 | 83493 | 18: 64684413 | 83500 | 18: 64871544 | 83506 | 18: 64979546 | 83507 | 18: 64989257 |
| 83508 | 18: 65000708 | 83512 | 18: 65024355 | 83513 | 18: 65052564 | 83514 | 18: 65058054 | 83515 | 18: 65058108 |
| 83516 | 18: 65058135 | 83522 | 18: 65143297 | 83538 | 18: 65213339 | 83546 | 18: 65269401 | 83570 | 18: 65467804 |
| 83573 | 18: 65499590 | 83576 | 18: 65531536 | 83582 | 18: 65680076 | 83584 | 18: 65778621 | 83611 | 19: 1044735 |
| 83641 | 19: 2028992 | 83643 | 19: 2087607 | 83647 | 19: 2189142 | 83648 | 19: 2189181 | 83669 | 19: 3019557 |
| 83676 | 19: 3224135 | 83684 | 19: 3507467 | 83688 | 19: 3693922 | 83689 | 19: 3693998 | 83690 | 19: 3694048 |
| 83691 | 19: 3717004 | 83712 | 19: 4248938 | 83719 | 19: 4532769 | 83732 | 19: 4885934 | 83737 | 19: 5057366 |
| 83738 | 19: 5077598 | 83742 | 19: 5131889 | 83743 | 19: 5144117 | 83748 | 19: 5302256 | 83758 | 19: 5544452 |
| 83759 | 19: 5550410 | 83770 | 19: 5841378 | 83773 | 19: 5911200 | 83777 | 19: 6008981 | 83779 | 19: 6069254 |
| 83780 | 19: 6080767 | 83802 | 19: 6741959 | 83803 | 19: 6742031 | 83804 | 19: 6744112 | 83808 | 19: 6821054 |
| 83810 | 19: 6854445 | 83823 | 19: 7084364 | 83825 | 19: 7139360 | 83830 | 19: 7250907 | 83835 | 19: 7311199 |
| 83857 | 19: 7610709 | 83860 | 19: 7655487 | 83867 | 19: 7877242 | 83870 | 19: 7955627 | 83874 | 19: 8012167 |
| 83882 | 19: 8102634 | 83884 | 19: 8134333 | 83888 | 19: 8242768 | 83890 | 19: 8251385 | 83891 | 19: 8253685 |
| 83892 | 19: 8253738 | 83893 | 19: 8256106 | 83894 | 19: 8257817 | 83896 | 19: 8303146 | 83897 | 19: 8307007 |
| 83899 | 19: 8318345 | 83909 | 19: 8375150 | 83914 | 19: 8447018 | 83917 | 19: 8481149 | 83919 | 19: 8538010 |
| 83928 | 19: 8786239 | 83937 | 19: 8921388 | 83938 | 19: 8940266 | 83939 | 19: 8940272 | 83946 | 19: 9148135 |
| 83948 | 19: 9161538 | 83951 | 19: 9186570 | 83952 | 19: 9186624 | 83953 | 19: 9186684 | 83954 | 19: 9186696 |
| 83955 | 19: 9186752 | 83960 | 19: 9321464 | 83963 | 19: 9325083 | 83964 | 19: 9329176 | 83971 | 19: 9494606 |
| 83973 | 19: 9527082 | 83974 | 19: 9549010 | 83976 | 19: 9584099 | 83978 | 19: 9638098 | 83996 | 19: 10144335 |
| 84000 | 19: 10161366 | 84001 | 19: 10185318 | 84002 | 19: 10193665 | 84004 | 19: 10203916 | 84005 | 19: 10212410 |
| 84006 | 19: 10222141 | 84012 | 19: 10311673 | 84015 | 19: 10357642 | 84018 | 19: 10410339 | 84027 | 19: 10608070 |
| 84028 | 19: 10663875 | 84033 | 19: 10816146 | 84048 | 19: 11195343 | 84049 | 19: 11206085 | 84050 | 19: 11223181 |
| 84051 | 19: 11223607 | 84074 | 19: 11794721 | 84081 | 19: 12080713 | 84098 | 19: 12520276 | 84106 | 19: 12624098 |
| 84115 | 19: 12851003 | 84118 | 19: 12942065 | 84119 | 19: 12942066 | 84138 | 19: 13385076 | 84153 | 19: 13539153 |
| 84154 | 19: 13540416 | 84156 | 19: 13546434 | 84157 | 19: 13553916 | 84158 | 19: 13554659 | 84160 | 19: 13567678 |
| 84161 | 19: 13567681 | 84162 | 19: 13569519 | 84163 | 19: 13589753 | 84167 | 19: 13627880 | 84168 | 19: 13629329 |
| 84182 | 19: 13922585 | 84184 | 19: 13992338 | 84185 | 19: 13992353 | 84193 | 19: 14098428 | 84197 | 19: 14234372 |
| 84199 | 19: 14285305 | 84202 | 19: 14359808 | 84206 | 19: 14455998 | 84207 | 19: 14458140 | 84208 | 19: 14460416 |
| 84209 | 19: 14461216 | 84212 | 19: 14524347 | 84215 | 19: 14594291 | 84231 | 19: 14987147 | 84233 | 19: 14997330 |
| 84236 | 19: 15044294 | 84238 | 19: 15079297 | 84265 | 19: 15653982 | 84275 | 19: 15950588 | 84282 | 19: 16153652 |
| 84285 | 19: 16179422 | 84286 | 19: 16179458 | 84290 | 19: 16243199 | 84291 | 19: 16245110 | 84294 | 19: 16316143 |
| 84296 | 19: 16414613 | 84297 | 19: 16414653 | 84298 | 19: 16414656 | 84299 | 19: 16414688 | 84300 | 19: 16414750 |
| 84301 | 19: 16414769 | 84305 | 19: 16538181 | 84306 | 19: 16538222 | 84307 | 19: 16538228 | 84308 | 19: 16538286 |
| 84309 | 19: 16538332 | 84314 | 19: 16680352 | 84315 | 19: 16698614 | 84316 | 19: 16701167 | 84317 | 19: 16704714 |
| 84318 | 19: 16710947 | 84319 | 19: 16712329 | 84320 | 19: 16715359 | 84329 | 19: 16900771 | 84344 | 19: 17139083 |
| 84345 | 19: 17193285 | 84347 | 19: 17219724 | 84354 | 19: 17346967 | 84358 | 19: 17413575 | 84360 | 19: 17439500 |
| 84361 | 19: 17463604 | 84362 | 19: 17463752 | 84363 | 19: 17463766 | 84364 | 19: 17463785 | 84366 | 19: 17476802 |
| 84367 | 19: 17485925 | 84381 | 19: 17839100 | 84382 | 19: 17840344 | 84383 | 19: 17847133 | 84388 | 19: 17935227 |
| 84390 | 19: 18002956 | 84391 | 19: 18012136 | 84405 | 19: 18364670 | 84427 | 19: 18812123 | 84429 | 19: 18935333 |
| 84434 | 19: 19027484 | 84438 | 19: 19066522 | 84440 | 19: 19164165 | 84441 | 19: 19167861 | 84443 | 19: 19248544 |
| 84448 | 19: 19342984 | 84455 | 19: 19497354 | 84456 | 19: 19503339 | 84469 | 19: 19832430 | 84470 | 19: 19832910 |
| 84473 | 19: 19891932 | 84478 | 19: 20010169 | 84479 | 19: 20010699 | 84480 | 19: 20021077 | 84481 | 19: 20024935 |
| 84482 | 19: 20039821 | 84483 | 19: 20039838 | 84484 | 19: 20049560 | 84486 | 19: 20064375 | 84487 | 19: 20065239 |
| 84488 | 19: 20065746 | 84489 | 19: 20067184 | 84490 | 19: 20067197 | 84496 | 19: 20169459 | 84497 | 19: 20252737 |
| 84498 | 19: 20269891 | 84499 | 19: 20272304 | 84500 | 19: 20272993 | 84501 | 19: 20272997 | 84502 | 19: 20287832 |
| 84503 | 19: 20347363 | 84504 | 19: 20347364 | 84505 | 19: 20347398 | 84506 | 19: 20347408 | 84507 | 19: 20347436 |
| 84508 | 19: 20347583 | 84511 | 19: 20397604 | 84513 | 19: 20404265 | 84514 | 19: 20406390 | 84515 | 19: 20407245 |
| 84516 | 19: 20407776 | 84517 | 19: 20407895 | 84518 | 19: 20408583 | 84519 | 19: 20409859 | 84520 | 19: 20410892 |
| 84521 | 19: 20411434 | 84522 | 19: 20412101 | 84523 | 19: 20413435 | 84524 | 19: 20414800 | 84525 | 19: 20415545 |
| 84526 | 19: 20415733 | 84527 | 19: 20416065 | 84528 | 19: 20416081 | 84529 | 19: 20416676 | 84530 | 19: 20417918 |
| 84531 | 19: 20418417 | 84532 | 19: 20418508 | 84533 | 19: 20418613 | 84535 | 19: 20421499 | 84536 | 19: 20423129 |
| 84537 | 19: 20427231 | 84538 | 19: 20443801 | 84539 | 19: 20448136 | 84540 | 19: 20449515 | 84541 | 19: 20450452 |
| 84542 | 19: 20451130 | 84543 | 19: 20454459 | 84544 | 19: 20459782 | 84545 | 19: 20464970 | 84546 | 19: 20466789 |
| 84547 | 19: 20468157 | 84548 | 19: 20469610 | 84557 | 19: 20885539 | 84561 | 19: 20948913 | 84570 | 19: 21194708 |
| 84571 | 19: 21195341 | 84580 | 19: 21541775 | 84582 | 19: 21606050 | 84583 | 19: 21606066 | 84584 | 19: 21606170 |
| 84586 | 19: 21607755 | 84587 | 19: 21607870 | 84588 | 19: 21657448 | 84596 | 19: 21761419 | 84601 | 19: 21810539 |
| 84602 | 19: 21810883 | 84608 | 19: 22007200 | 84613 | 19: 22125908 | 84617 | 19: 22209844 | 84618 | 19: 22216194 |
| 84619 | 19: 22216936 | 84620 | 19: 22218946 | 84621 | 19: 22223761 | 84622 | 19: 22225302 | 84624 | 19: 22228117 |
| 84625 | 19: 22228881 | 84626 | 19: 22238525 | 84629 | 19: 22288784 | 84632 | 19: 22387067 | 84637 | 19: 22435985 |
| 84640 | 19: 22580315 | 84642 | 19: 22630921 | 84645 | 19: 22686577 | 84649 | 19: 22777911 | 84653 | 19: 22919648 |
| 84654 | 19: 22933829 | 84655 | 19: 22937788 | 84659 | 19: 23047864 | 84663 | 19: 23089782 | 84665 | 19: 23103859 |
| 84666 | 19: 23125220 | 84677 | 19: 23322846 | 84678 | 19: 23328857 | 84691 | 19: 23748456 | 84692 | 19: 23762942 |
| 84696 | 19: 23795890 | 84699 | 19: 23844740 | 84709 | 19: 24085976 | 84710 | 19: 24086312 | 84711 | 19: 24086389 |
| 84712 | 19: 24100573 | 84713 | 19: 24119941 | 84714 | 19: 24120015 | 84715 | 19: 24120089 | 84716 | 19: 24120654 |
| 84717 | 19: 24120726 | 84719 | 19: 24158070 | 84720 | 19: 24171676 | 84721 | 19: 24190844 | 84722 | 19: 24190846 |
| 84723 | 19: 24190851 | 84724 | 19: 24190893 | 84725 | 19: 24201030 | 84726 | 19: 24201096 | 84727 | 19: 24203359 |
| 84728 | 19: 24203402 | 84729 | 19: 24203454 | 84730 | 19: 24204657 | 84731 | 19: 24206255 | 84732 | 19: 24206362 |
| 84733 | 19: 24210049 | 84734 | 19: 24211193 | 84740 | 19: 24352581 | 84749 | 19: 24484569 | 84752 | 19: 24575685 |
| 84757 | 19: 24628286 | 84758 | 19: 24643342 | 84760 | 19: 24654902 | 84763 | 19: 24710033 | 84766 | 19: 24729440 |
| 84767 | 19: 24730057 | 84768 | 19: 24732020 | 84769 | 19: 24736477 | 84770 | 19: 24737388 | 84771 | 19: 24739849 |
| 84772 | 19: 24743924 | 84773 | 19: 24747961 | 84774 | 19: 24753662 | 84776 | 19: 24761701 | 84777 | 19: 24791465 |
| 84778 | 19: 24832457 | 84779 | 19: 24834421 | 84780 | 19: 24843420 | 84782 | 19: 24848682 | 84783 | 19: 24859742 |
| 84784 | 19: 24862477 | 84787 | 19: 24938845 | 84788 | 19: 24958116 | 84789 | 19: 24958789 | 84793 | 19: 25015319 |
| 84795 | 19: 25042003 | 84797 | 19: 25049455 | 84799 | 19: 25055788 | 84802 | 19: 25112769 | 84808 | 19: 25148679 |
| 84809 | 19: 25149070 | 84810 | 19: 25167024 | 84815 | 19: 25292038 | 84843 | 19: 25636984 | 84847 | 19: 25714307 |
| 84866 | 19: 26042854 | 84867 | 19: 26047036 | 84868 | 19: 26047065 | 84869 | 19: 26047497 | 84870 | 19: 26047588 |
| 84871 | 19: 26048186 | 84872 | 19: 26048187 | 84873 | 19: 26048200 | 84874 | 19: 26048237 | 84875 | 19: 26048249 |
| 84876 | 19: 26050069 | 84877 | 19: 26050076 | 84878 | 19: 26050122 | 84894 | 19: 26326260 | 84895 | 19: 26328504 |
| 84896 | 19: 26330382 | 84897 | 19: 26362443 | 84903 | 19: 26386801 | 84909 | 19: 26431555 | 84911 | 19: 26433035 |
| 84913 | 19: 26435773 | 84914 | 19: 26437827 | 84915 | 19: 26438388 | 84919 | 19: 26443714 | 84920 | 19: 26452130 |
| 84921 | 19: 26455955 | 84923 | 19: 26459081 | 84924 | 19: 26461611 | 84929 | 19: 26467372 | 84933 | 19: 26494500 |
| 84934 | 19: 26498170 | 84937 | 19: 26508308 | 84938 | 19: 26509449 | 84940 | 19: 26512520 | 84943 | 19: 26571898 |
| 84967 | 19: 26833517 | 84968 | 19: 26834363 | 84971 | 19: 26944800 | 84972 | 19: 26944904 | 84991 | 19: 27851735 |
| 84994 | 19: 27919465 | 84995 | 19: 27929802 | 84997 | 19: 27974645 | 84999 | 19: 27986943 | 85003 | 19: 28063061 |
| 85022 | 19: 28341985 | 85027 | 19: 28441795 | 85033 | 19: 28599438 | 85039 | 19: 28789212 | 85055 | 19: 29142555 |
| 85056 | 19: 29146537 | 85057 | 19: 29165645 | 85060 | 19: 29185159 | 85062 | 19: 29191189 | 85070 | 19: 29360624 |
| 85071 | 19: 29360635 | 85077 | 19: 29516407 | 85082 | 19: 29601656 | 85088 | 19: 29667582 | 85089 | 19: 29671837 |
| 85093 | 19: 29784730 | 85094 | 19: 29784751 | 85095 | 19: 29784815 | 85106 | 19: 30101489 | 85109 | 19: 30199565 |
| 85125 | 19: 30493276 | 85143 | 19: 30892000 | 85145 | 19: 30952848 | 85148 | 19: 31087410 | 85150 | 19: 31146132 |
| 85153 | 19: 31161313 | 85154 | 19: 31165405 | 85155 | 19: 31165486 | 85160 | 19: 31346087 | 85161 | 19: 31369446 |
| 85193 | 19: 32163965 | 85194 | 19: 32172115 | 85201 | 19: 32267525 | 85223 | 19: 32751602 | 85224 | 19: 32768693 |
| 85225 | 19: 32800795 | 85226 | 19: 32804878 | 85233 | 19: 33007630 | 85234 | 19: 33019809 | 85238 | 19: 33125153 |
| 85243 | 19: 33187115 | 85246 | 19: 33215711 | 85249 | 19: 33262257 | 85255 | 19: 33294100 | 85267 | 19: 33578331 |
| 85268 | 19: 33594931 | 85269 | 19: 33596340 | 85271 | 19: 33623310 | 85279 | 19: 33954621 | 85281 | 19: 33998092 |
| 85282 | 19: 34005543 | 85283 | 19: 34005879 | 85286 | 19: 34041049 | 85303 | 19: 34565878 | 85306 | 19: 34576319 |
| 85313 | 19: 34769105 | 85314 | 19: 34779947 | 85315 | 19: 34782489 | 85316 | 19: 34786152 | 85317 | 19: 34795515 |
| 85318 | 19: 34801199 | 85319 | 19: 34814559 | 85320 | 19: 34815409 | 85321 | 19: 34821534 | 85322 | 19: 34822575 |
| 85323 | 19: 34823488 | 85324 | 19: 34824385 | 85325 | 19: 34825185 | 85326 | 19: 34826088 | 85327 | 19: 34826902 |
| 85328 | 19: 34827822 | 85329 | 19: 34832510 | 85330 | 19: 34833809 | 85331 | 19: 34835066 | 85332 | 19: 34839003 |
| 85333 | 19: 34839771 | 85334 | 19: 34840668 | 85335 | 19: 34842069 | 85336 | 19: 34843150 | 85337 | 19: 34850412 |
| 85338 | 19: 34851901 | 85339 | 19: 34860953 | 85340 | 19: 34867998 | 85341 | 19: 34873374 | 85342 | 19: 34876608 |
| 85343 | 19: 34877505 | 85344 | 19: 34884414 | 85346 | 19: 34893695 | 85348 | 19: 34911757 | 85349 | 19: 34917689 |
| 85350 | 19: 34924937 | 85351 | 19: 34940577 | 85352 | 19: 34941647 | 85353 | 19: 34942605 | 85360 | 19: 35015390 |
| 85385 | 19: 35630509 | 85391 | 19: 35778297 | 85404 | 19: 36050548 | 85405 | 19: 36052759 | 85408 | 19: 36139902 |
| 85410 | 19: 36161222 | 85412 | 19: 36183218 | 85413 | 19: 36186920 | 85414 | 19: 36204759 | 85415 | 19: 36205924 |
| 85417 | 19: 36230644 | 85418 | 19: 36232203 | 85419 | 19: 36233310 | 85420 | 19: 36234144 | 85421 | 19: 36235392 |
| 85422 | 19: 36236022 | 85423 | 19: 36236961 | 85424 | 19: 36238112 | 85425 | 19: 36239325 | 85426 | 19: 36242008 |
| 85430 | 19: 36285683 | 85432 | 19: 36304660 | 85436 | 19: 36349079 | 85443 | 19: 36515692 | 85444 | 19: 36516381 |
| 85445 | 19: 36540830 | 85446 | 19: 36572979 | 85447 | 19: 36574100 | 85448 | 19: 36574859 | 85449 | 19: 36574986 |
| 85450 | 19: 36576060 | 85451 | 19: 36577180 | 85452 | 19: 36577974 | 85453 | 19: 36585052 | 85454 | 19: 36585096 |
| 85455 | 19: 36585153 | 85456 | 19: 36586555 | 85457 | 19: 36588245 | 85458 | 19: 36598037 | 85460 | 19: 36659503 |
| 85478 | 19: 37057391 | 85480 | 19: 37085911 | 85503 | 19: 37768695 | 85512 | 19: 38027080 | 85513 | 19: 38112442 |
| 85514 | 19: 38115441 | 85515 | 19: 38135659 | 85519 | 19: 38330215 | 85523 | 19: 38378249 | 85535 | 19: 38818642 |
| 85537 | 19: 38825404 | 85543 | 19: 39018067 | 85546 | 19: 39173581 | 85548 | 19: 39195126 | 85554 | 19: 39319605 |
| 85555 | 19: 39319939 | 85556 | 19: 39324677 | 85557 | 19: 39373787 | 85558 | 19: 39403214 | 85560 | 19: 39423726 |
| 85564 | 19: 39558542 | 85568 | 19: 39592912 | 85572 | 19: 39664178 | 85574 | 19: 39702056 | 85575 | 19: 39761996 |
| 85585 | 19: 40136281 | 85589 | 19: 40231461 | 85591 | 19: 40243945 | 85592 | 19: 40244440 | 85593 | 19: 40244746 |
| 85594 | 19: 40250106 | 85603 | 19: 40340447 | 85608 | 19: 40504497 | 85611 | 19: 40571544 | 85617 | 19: 40687629 |
| 85634 | 19: 40969791 | 85636 | 19: 40998269 | 85637 | 19: 41009813 | 85639 | 19: 41075395 | 85641 | 19: 41125615 |
| 85642 | 19: 41125654 | 85643 | 19: 41126901 | 85644 | 19: 41134261 | 85645 | 19: 41134335 | 85646 | 19: 41167136 |
| 85666 | 19: 41672120 | 85671 | 19: 41759325 | 85681 | 19: 41844566 | 85683 | 19: 41866585 | 85685 | 19: 41908524 |
| 85687 | 19: 41944555 | 85689 | 19: 41954155 | 85694 | 19: 42058566 | 85695 | 19: 42063521 | 85698 | 19: 42131015 |
| 85701 | 19: 42144465 | 85704 | 19: 42227260 | 85710 | 19: 42333202 | 85712 | 19: 42345437 | 85719 | 19: 42426465 |
| 85728 | 19: 42644624 | 85731 | 19: 42719942 | 85732 | 19: 42726195 | 85736 | 19: 42848843 | 85738 | 19: 42887598 |
| 85741 | 19: 43035501 | 85742 | 19: 43043947 | 85743 | 19: 43049014 | 85750 | 19: 43333514 | 85759 | 19: 43422824 |
| 85768 | 19: 43443947 | 85775 | 19: 43599080 | 85777 | 19: 43713425 | 85783 | 19: 43816258 | 85784 | 19: 43830215 |
| 85787 | 19: 43926648 | 85790 | 19: 43991252 | 85793 | 19: 44072509 | 85795 | 19: 44116524 | 85797 | 19: 44122310 |
| 85799 | 19: 44173520 | 85800 | 19: 44177664 | 85801 | 19: 44178974 | 85806 | 19: 44309222 | 85808 | 19: 44359879 |
| 85809 | 19: 44385146 | 85813 | 19: 44477082 | 85818 | 19: 44577139 | 85821 | 19: 44615861 | 85822 | 19: 44651270 |
| 85823 | 19: 44673344 | 85824 | 19: 44673867 | 85826 | 19: 44715813 | 85838 | 19: 45013727 | 85841 | 19: 45073651 |
| 85861 | 19: 45630233 | 85892 | 19: 45816366 | 85893 | 19: 45817041 | 85896 | 19: 45844326 | 85898 | 19: 45920605 |
| 85899 | 19: 45934999 | 85900 | 19: 45949710 | 85909 | 19: 46106226 | 85918 | 19: 46396057 | 85928 | 19: 46762829 |
| 85933 | 19: 46867389 | 85944 | 19: 47138439 | 85945 | 19: 47138441 | 85946 | 19: 47138654 | 85947 | 19: 47138655 |
| 85948 | 19: 47138674 | 85949 | 19: 47138684 | 85950 | 19: 47139281 | 85958 | 19: 47384175 | 85959 | 19: 47402508 |
| 85960 | 19: 47405017 | 85974 | 19: 47676904 | 85975 | 19: 47735369 | 85979 | 19: 47763256 | 85984 | 19: 47930272 |
| 85986 | 19: 47955876 | 85987 | 19: 47966555 | 85990 | 19: 48025233 | 85993 | 19: 48071339 | 85994 | 19: 48075239 |
| 85996 | 19: 48084284 | 86000 | 19: 48118256 | 86001 | 19: 48142220 | 86005 | 19: 48258274 | 86006 | 19: 48266963 |
| 86012 | 19: 48389397 | 86016 | 19: 48418566 | 86022 | 19: 48563047 | 86028 | 19: 48709127 | 86032 | 19: 48880795 |
| 86042 | 19: 49040357 | 86051 | 19: 49233318 | 86055 | 19: 49277856 | 86063 | 19: 49478759 | 86064 | 19: 49483816 |
| 86065 | 19: 49494727 | 86068 | 19: 49575523 | 86076 | 19: 49772893 | 86078 | 19: 49810028 | 86085 | 19: 49891419 |
| 86092 | 19: 50063999 | 86094 | 19: 50095971 | 86095 | 19: 50107587 | 86096 | 19: 50123299 | 86099 | 19: 50167379 |
| 86100 | 19: 50172634 | 86101 | 19: 50173024 | 86102 | 19: 50173036 | 86103 | 19: 50173360 | 86104 | 19: 50173420 |
| 86116 | 19: 50607587 | 86118 | 19: 50662334 | 86122 | 19: 50682193 | 86139 | 19: 50783155 | 86140 | 19: 50784393 |
| 86142 | 19: 50788319 | 86144 | 19: 50790149 | 86145 | 19: 50790973 | 86146 | 19: 50791045 | 86147 | 19: 50817214 |
| 86148 | 19: 50817251 | 86150 | 19: 50838297 | 86151 | 19: 50838402 | 86152 | 19: 50855700 | 86153 | 19: 50856195 |
| 86154 | 19: 50883463 | 86155 | 19: 50890139 | 86156 | 19: 50892965 | 86157 | 19: 50893275 | 86158 | 19: 50893330 |
| 86159 | 19: 50893412 | 86160 | 19: 50894507 | 86164 | 19: 51146306 | 86165 | 19: 51148044 | 86166 | 19: 51150289 |
| 86167 | 19: 51151634 | 86168 | 19: 51152336 | 86169 | 19: 51152846 | 86170 | 19: 51153433 | 86171 | 19: 51155662 |
| 86172 | 19: 51156301 | 86173 | 19: 51158110 | 86174 | 19: 51172862 | 86175 | 19: 51173451 | 86176 | 19: 51179705 |
| 86177 | 19: 51181597 | 86178 | 19: 51184543 | 86179 | 19: 51185390 | 86180 | 19: 51186411 | 86181 | 19: 51186781 |
| 86182 | 19: 51186933 | 86183 | 19: 51187678 | 86184 | 19: 51192006 | 86185 | 19: 51194044 | 86186 | 19: 51195370 |
| 86187 | 19: 51196420 | 86188 | 19: 51197290 | 86189 | 19: 51197844 | 86190 | 19: 51198485 | 86191 | 19: 51200087 |
| 86192 | 19: 51202775 | 86193 | 19: 51203906 | 86194 | 19: 51204912 | 86195 | 19: 51206147 | 86196 | 19: 51206982 |
| 86197 | 19: 51208278 | 86198 | 19: 51209192 | 86199 | 19: 51215165 | 86213 | 19: 51584353 | 86214 | 19: 51614996 |
| 86215 | 19: 51624830 | 86219 | 19: 51657080 | 86220 | 19: 51674934 | 86221 | 19: 51674957 | 86222 | 19: 51674972 |
| 86223 | 19: 51676635 | 86225 | 19: 51688593 | 86226 | 19: 51689866 | 86238 | 19: 52025573 | 86243 | 19: 52232662 |
| 86246 | 19: 52242008 | 86261 | 19: 52716129 | 86275 | 19: 52957714 | 86277 | 19: 53006255 | 86279 | 19: 53041756 |
| 86281 | 19: 53068039 | 86285 | 19: 53125990 | 86286 | 19: 53132946 | 86301 | 19: 53433970 | 86302 | 19: 53439232 |
| 86304 | 19: 53484305 | 86308 | 19: 53566656 | 86309 | 19: 53572790 | 86310 | 19: 53588198 | 86314 | 19: 53698415 |
| 86318 | 19: 53748765 | 86320 | 19: 53774472 | 86321 | 19: 53778116 | 86323 | 19: 53811161 | 86333 | 19: 53948075 |
| 86337 | 19: 54028347 | 86338 | 19: 54043604 | 86341 | 19: 54053040 | 86352 | 19: 54289712 | 86353 | 19: 54295559 |
| 86355 | 19: 54301176 | 86358 | 19: 54390593 | 86371 | 19: 54622173 | 86372 | 19: 54637181 | 86379 | 19: 54710734 |
| 86383 | 19: 54755988 | 86388 | 19: 54858324 | 86393 | 19: 54978600 | 86398 | 19: 55092867 | 86403 | 19: 55229692 |
| 86406 | 19: 55265827 | 86409 | 19: 55330553 | 86413 | 19: 55427978 | 86414 | 19: 55428029 | 86422 | 19: 55580085 |
| 86427 | 19: 55698969 | 86432 | 19: 55792180 | 86433 | 19: 55793483 | 86434 | 19: 55793574 | 86436 | 19: 55827900 |
| 86437 | 19: 55830150 | 86440 | 19: 55848361 | 86441 | 19: 55848663 | 86444 | 19: 55866969 | 86449 | 19: 55906352 |
| 86452 | 19: 55911397 | 86454 | 19: 55915911 | 86455 | 19: 55917441 | 86456 | 19: 55920727 | 86458 | 19: 55922694 |
| 86462 | 19: 55926659 | 86464 | 19: 55940240 | 86471 | 19: 55985092 | 86472 | 19: 55985151 | 86473 | 19: 55985483 |
| 86474 | 19: 55986867 | 86475 | 19: 55986948 | 86477 | 19: 55987569 | 86479 | 19: 55988850 | 86480 | 19: 55988905 |
| 86481 | 19: 55988950 | 86482 | 19: 55989161 | 86483 | 19: 55989317 | 86484 | 19: 55989324 | 86486 | 19: 55993442 |
| 86489 | 19: 56009506 | 86495 | 19: 56024722 | 86500 | 19: 56044293 | 86503 | 19: 56059597 | 86512 | 19: 56100545 |
| 86516 | 19: 56104113 | 86535 | 19: 56318677 | 86536 | 19: 56320176 | 86537 | 19: 56346313 | 86538 | 19: 56346447 |
| 86539 | 19: 56348743 | 86540 | 19: 56388703 | 86544 | 19: 56447130 | 86549 | 19: 56504478 | 86550 | 19: 56506885 |
| 86551 | 19: 56506918 | 86553 | 19: 56521721 | 86554 | 19: 56527708 | 86556 | 19: 56536304 | 86558 | 19: 56542031 |
| 86559 | 19: 56544120 | 86560 | 19: 56545458 | 86561 | 19: 56546864 | 86562 | 19: 56553455 | 86571 | 19: 56792481 |
| 86573 | 19: 56794455 | 86576 | 19: 56891022 | 86579 | 19: 56962292 | 86581 | 19: 57011124 | 86582 | 19: 57012570 |
| 86591 | 19: 57196446 | 86592 | 19: 57214845 | 86593 | 19: 57259057 | 86600 | 19: 57418240 | 86608 | 19: 57566385 |
| 86631 | 19: 58091496 | 86632 | 19: 58091529 | 86634 | 19: 58127830 | 86638 | 19: 58214009 | 86639 | 19: 58218020 |
| 86645 | 19: 58304430 | 86650 | 19: 58429115 | 86659 | 19: 58594514 | 86665 | 19: 58750938 | 86673 | 19: 58918004 |
| 86688 | 19: 59274219 | 86689 | 19: 59281932 | 86711 | 19: 59592874 | 86718 | 19: 59660094 | 86724 | 19: 59743571 |
| 86727 | 19: 59784192 | 86731 | 19: 59871909 | 86732 | 19: 59872022 | 86742 | 19: 59984597 | 86756 | 19: 60173328 |
| 86768 | 19: 60319323 | 86776 | 19: 60416386 | 86781 | 19: 60493532 | 86782 | 19: 60495581 | 86793 | 19: 60643681 |
| 86795 | 19: 60660851 | 86800 | 19: 60743931 | 86802 | 19: 60816246 | 86811 | 19: 60977087 | 86814 | 19: 61065143 |
| 86818 | 19: 61145338 | 86819 | 19: 61146374 | 86822 | 19: 61175458 | 86823 | 19: 61175494 | 86824 | 19: 61175495 |
| 86825 | 19: 61175593 | 86826 | 19: 61175611 | 86834 | 19: 61242337 | 86845 | 19: 61459019 | 86846 | 19: 61474017 |
| 86848 | 19: 61529467 | 86849 | 19: 61531654 | 86850 | 19: 61531677 | 86851 | 19: 61532390 | 86852 | 19: 61533739 |
| 86854 | 19: 61538770 | 86862 | 19: 61639102 | 86865 | 19: 61681705 | 86867 | 19: 61701942 | 86868 | 19: 61709842 |
| 86874 | 19: 61794791 | 86884 | 19: 61956159 | 86885 | 19: 61968113 | 86892 | 19: 62135323 | 86904 | 19: 62322264 |
| 86915 | 19: 62611634 | 86916 | 19: 62620305 | 86919 | 19: 62738018 | 86927 | 19: 62774324 | 86928 | 19: 62785952 |
| 86942 | 19: 63013941 | 86944 | 19: 63088763 | 86946 | 19: 63091044 | 86959 | 19: 63394562 | 86960 | 19: 63394839 |
| 86965 | 20: 283185 | 86975 | 20: 495073 | 86979 | 20: 523765 | 86989 | 20: 666472 | 87009 | 20: 1104356 |
| 87012 | 20: 1164612 | 87016 | 20: 1365586 | 87019 | 20: 1476960 | 87020 | 20: 1477326 | 87025 | 20: 1585296 |
| 87031 | 20: 1627743 | 87032 | 20: 1627820 | 87033 | 20: 1628846 | 87038 | 20: 1708322 | 87040 | 20: 1708393 |
| 87051 | 20: 1881180 | 87054 | 20: 1890880 | 87058 | 20: 1964788 | 87059 | 20: 1981954 | 87060 | 20: 1985087 |
| 87062 | 20: 2008634 | 87066 | 20: 2111352 | 87071 | 20: 2174269 | 87082 | 20: 2329012 | 87084 | 20: 2363600 |
| 87090 | 20: 2486996 | 87093 | 20: 2549449 | 87094 | 20: 2549482 | 87123 | 20: 3122350 | 87125 | 20: 3132189 |
| 87126 | 20: 3144963 | 87127 | 20: 3147090 | 87128 | 20: 3149103 | 87133 | 20: 3256361 | 87141 | 20: 3434776 |
| 87143 | 20: 3442837 | 87144 | 20: 3490573 | 87153 | 20: 3643964 | 87163 | 20: 3970013 | 87184 | 20: 4419380 |
| 87186 | 20: 4422606 | 87187 | 20: 4422634 | 87189 | 20: 4471095 | 87201 | 20: 4600219 | 87353 | 20: 4974566 |
| 87356 | 20: 4978213 | 87396 | 20: 5081279 | 87397 | 20: 5081368 | 87398 | 20: 5081429 | 87411 | 20: 5256314 |
| 87424 | 20: 5427984 | 87425 | 20: 5449185 | 87427 | 20: 5456791 | 87445 | 20: 5952561 | 87471 | 20: 6124117 |
| 87472 | 20: 6127051 | 87473 | 20: 6128007 | 87474 | 20: 6129222 | 87475 | 20: 6135444 | 87478 | 20: 6222819 |
| 87491 | 20: 6538447 | 87493 | 20: 6552399 | 87502 | 20: 6787304 | 87503 | 20: 6803666 | 87504 | 20: 6804954 |
| 87505 | 20: 6809070 | 87507 | 20: 6819134 | 87508 | 20: 6824958 | 87514 | 20: 7026132 | 87515 | 20: 7028111 |
| 87527 | 20: 7184948 | 87529 | 20: 7203346 | 87530 | 20: 7228830 | 87536 | 20: 7397715 | 87538 | 20: 7452989 |
| 87556 | 20: 7937085 | 87558 | 20: 7966207 | 87565 | 20: 8099607 | 87580 | 20: 8406030 | 87588 | 20: 8596907 |
| 87591 | 20: 8676605 | 87597 | 20: 8915848 | 87599 | 20: 8976316 | 87600 | 20: 8994477 | 87603 | 20: 9008027 |
| 87604 | 20: 9008070 | 87610 | 20: 9122965 | 87611 | 20: 9185096 | 87616 | 20: 9277064 | 87617 | 20: 9284840 |
| 87618 | 20: 9286211 | 87619 | 20: 9286666 | 87620 | 20: 9297979 | 87622 | 20: 9309443 | 87627 | 20: 9387110 |
| 87635 | 20: 9562025 | 87642 | 20: 9678407 | 87643 | 20: 9699294 | 87653 | 20: 9987753 | 87657 | 20: 10105910 |
| 87670 | 20: 10568285 | 87677 | 20: 10719241 | 87691 | 20: 11104981 | 87695 | 20: 11173739 | 87708 | 20: 11384109 |
| 87709 | 20: 11423657 | 87712 | 20: 11441654 | 87719 | 20: 11603501 | 87723 | 20: 11652136 | 87726 | 20: 11746445 |
| 87727 | 20: 11748411 | 87729 | 20: 11801270 | 87732 | 20: 11873589 | 87736 | 20: 12001911 | 87745 | 20: 12195219 |
| 87751 | 20: 12336592 | 87756 | 20: 12431016 | 87759 | 20: 12454756 | 87760 | 20: 12470759 | 87770 | 20: 12619125 |
| 87776 | 20: 12725086 | 87777 | 20: 12732314 | 87787 | 20: 12872002 | 87788 | 20: 12884968 | 87792 | 20: 13038217 |
| 87800 | 20: 13212537 | 87804 | 20: 13314745 | 87806 | 20: 13360616 | 87808 | 20: 13384250 | 87809 | 20: 13411239 |
| 87820 | 20: 13846644 | 87821 | 20: 13853397 | 87827 | 20: 13967913 | 87881 | 20: 16013654 | 87890 | 20: 16311925 |
| 87891 | 20: 16312024 | 87892 | 20: 16312059 | 87893 | 20: 16312062 | 87926 | 20: 17161518 | 87940 | 20: 17529786 |
| 87946 | 20: 17718360 | 87949 | 20: 17740634 | 87950 | 20: 17772296 | 87959 | 20: 18106764 | 87966 | 20: 18199987 |
| 87974 | 20: 18560092 | 87979 | 20: 18738258 | 87981 | 20: 18767430 | 87988 | 20: 18999163 | 87998 | 20: 19219710 |
| 88010 | 20: 19525255 | 88013 | 20: 19638381 | 88024 | 20: 19910542 | 88026 | 20: 19934333 | 88029 | 20: 19951507 |
| 88030 | 20: 19985004 | 88035 | 20: 20229781 | 88049 | 20: 20735095 | 88053 | 20: 20833870 | 88054 | 20: 20847713 |
| 88055 | 20: 20852713 | 88057 | 20: 20900999 | 88058 | 20: 20916403 | 88060 | 20: 20932871 | 88062 | 20: 20985150 |
| 88069 | 20: 21165648 | 88070 | 20: 21199073 | 88076 | 20: 21442552 | 88119 | 20: 22069390 | 88123 | 20: 22130295 |
| 88158 | 20: 22204908 | 88186 | 20: 22465821 | 88204 | 20: 22827690 | 88213 | 20: 23009076 | 88214 | 20: 23038319 |
| 88215 | 20: 23039137 | 88224 | 20: 23272824 | 88234 | 20: 23534898 | 88236 | 20: 23571173 | 88237 | 20: 23574812 |
| 88238 | 20: 23575304 | 88242 | 20: 23686099 | 88243 | 20: 23692874 | 88248 | 20: 23836890 | 88249 | 20: 23837832 |
| 88250 | 20: 23838131 | 88251 | 20: 23838181 | 88252 | 20: 23841545 | 88253 | 20: 23861537 | 88254 | 20: 23861790 |
| 88255 | 20: 23862521 | 88256 | 20: 23863171 | 88257 | 20: 23864163 | 88258 | 20: 23864320 | 88260 | 20: 23868037 |
| 88261 | 20: 23872171 | 88262 | 20: 23872177 | 88263 | 20: 23873363 | 88265 | 20: 23931936 | 88266 | 20: 23934587 |
| 88267 | 20: 23936814 | 88268 | 20: 23937494 | 88269 | 20: 23940208 | 88270 | 20: 23940805 | 88271 | 20: 23948706 |
| 88272 | 20: 23950019 | 88273 | 20: 23956567 | 88274 | 20: 23967747 | 88278 | 20: 24005106 | 88283 | 20: 24183792 |
| 88289 | 20: 24300670 | 88291 | 20: 24342520 | 88301 | 20: 24551449 | 88303 | 20: 24600379 | 88304 | 20: 24644625 |
| 88314 | 20: 24840568 | 88316 | 20: 24888410 | 88336 | 20: 25687633 | 88341 | 20: 25956846 | 88353 | 20: 26326568 |
| 88377 | 20: 26904319 | 88394 | 20: 27389017 | 88396 | 20: 27389119 | 88418 | 20: 27942740 | 88423 | 20: 28062228 |
| 88424 | 20: 28062230 | 88425 | 20: 28067347 | 88435 | 20: 28291529 | 88440 | 20: 28407293 | 88449 | 20: 28594465 |
| 88452 | 20: 28714693 | 88453 | 20: 28719941 | 88454 | 20: 28719958 | 88455 | 20: 28720052 | 88456 | 20: 28726509 |
| 88457 | 20: 28726541 | 88458 | 20: 28726553 | 88459 | 20: 28726570 | 88460 | 20: 28726626 | 88468 | 20: 28835792 |
| 88474 | 20: 28977237 | 88475 | 20: 28978238 | 88488 | 20: 29362772 | 88489 | 20: 29376013 | 88491 | 20: 29447835 |
| 88508 | 20: 29990492 | 88509 | 20: 29990621 | 88514 | 20: 30118413 | 88518 | 20: 30200252 | 88520 | 20: 30212011 |
| 88532 | 20: 30669895 | 88536 | 20: 30689871 | 88539 | 20: 30825518 | 88546 | 20: 31205981 | 88547 | 20: 31211377 |
| 88548 | 20: 31243755 | 88549 | 20: 31245339 | 88558 | 20: 31456913 | 88567 | 20: 31684137 | 88570 | 20: 31752397 |
| 88576 | 20: 31881419 | 88579 | 20: 31903713 | 88582 | 20: 31972125 | 88585 | 20: 32052139 | 88588 | 20: 32101132 |
| 88597 | 20: 32409103 | 88633 | 20: 33398481 | 88637 | 20: 33502768 | 88640 | 20: 33565104 | 88641 | 20: 33569466 |
| 88648 | 20: 33719114 | 88653 | 20: 33836807 | 88657 | 20: 34001523 | 88663 | 20: 34145466 | 88666 | 20: 34184342 |
| 88669 | 20: 34272037 | 88692 | 20: 34738766 | 88694 | 20: 34781170 | 88697 | 20: 34913079 | 88714 | 20: 35421048 |
| 88721 | 20: 35551560 | 88722 | 20: 35585029 | 88723 | 20: 35610755 | 88726 | 20: 35701545 | 88728 | 20: 35715693 |
| 88736 | 20: 35846193 | 88737 | 20: 35882733 | 88741 | 20: 35994055 | 88742 | 20: 35994063 | 88743 | 20: 35996784 |
| 88762 | 20: 36319426 | 88767 | 20: 36496636 | 88776 | 20: 36648116 | 88781 | 20: 36787045 | 88782 | 20: 36834159 |
| 88783 | 20: 36858954 | 88785 | 20: 36892081 | 88788 | 20: 36968962 | 88789 | 20: 36989703 | 88791 | 20: 37042637 |
| 88794 | 20: 37085325 | 88795 | 20: 37092573 | 88797 | 20: 37131660 | 88798 | 20: 37131690 | 88799 | 20: 37136413 |
| 88804 | 20: 37341795 | 88810 | 20: 37443371 | 88811 | 20: 37443444 | 88812 | 20: 37443481 | 88813 | 20: 37473672 |
| 88815 | 20: 37479797 | 88816 | 20: 37481956 | 88827 | 20: 37694264 | 88828 | 20: 37694333 | 88831 | 20: 37811388 |
| 88832 | 20: 37811427 | 88833 | 20: 37811480 | 88834 | 20: 37811543 | 88838 | 20: 37887781 | 88845 | 20: 38177941 |
| 88846 | 20: 38189067 | 88867 | 20: 38758576 | 88881 | 20: 39088930 | 88882 | 20: 39109157 | 88887 | 20: 39238537 |
| 88888 | 20: 39245057 | 88890 | 20: 39287657 | 88893 | 20: 39383171 | 88894 | 20: 39383264 | 88898 | 20: 39445232 |
| 88904 | 20: 39475943 | 88909 | 20: 39539960 | 88911 | 20: 39555296 | 88912 | 20: 39557649 | 88924 | 20: 39810901 |
| 88925 | 20: 39863071 | 88928 | 20: 39910970 | 88930 | 20: 39965197 | 88937 | 20: 40059831 | 88941 | 20: 40215915 |
| 88955 | 20: 40683858 | 88956 | 20: 40684807 | 88957 | 20: 40695016 | 88961 | 20: 40772462 | 88966 | 20: 40834506 |
| 88968 | 20: 40945725 | 88975 | 20: 41050907 | 88976 | 20: 41050966 | 88978 | 20: 41100712 | 88984 | 20: 41160179 |
| 88986 | 20: 41192863 | 88990 | 20: 41314439 | 89010 | 20: 41798915 | 89012 | 20: 41819893 | 89013 | 20: 41823837 |
| 89016 | 20: 41845476 | 89023 | 20: 41919178 | 89028 | 20: 42044095 | 89029 | 20: 42045334 | 89032 | 20: 42085412 |
| 89035 | 20: 42133834 | 89039 | 20: 42246244 | 89042 | 20: 42293890 | 89043 | 20: 42331127 | 89047 | 20: 42565028 |
| 89061 | 20: 42942479 | 89066 | 20: 43159533 | 89079 | 20: 43561846 | 89080 | 20: 43561856 | 89101 | 20: 44025947 |
| 89102 | 20: 44026070 | 89107 | 20: 44281699 | 89108 | 20: 44301844 | 89121 | 20: 44556774 | 89122 | 20: 44574229 |
| 89123 | 20: 44591612 | 89133 | 20: 44966449 | 89134 | 20: 44969446 | 89145 | 20: 45365363 | 89186 | 20: 46291117 |
| 89194 | 20: 46578492 | 89208 | 20: 47160581 | 89209 | 20: 47160583 | 89211 | 20: 47163202 | 89212 | 20: 47163219 |
| 89243 | 20: 48405434 | 89244 | 20: 48405481 | 89245 | 20: 48405501 | 89246 | 20: 48405505 | 89247 | 20: 48405507 |
| 89248 | 20: 48405524 | 89249 | 20: 48405575 | 89251 | 20: 48474578 | 89262 | 20: 48874528 | 89264 | 20: 48881242 |
| 89265 | 20: 48881303 | 89266 | 20: 48881343 | 89267 | 20: 48901928 | 89274 | 20: 49054367 | 89297 | 20: 49861661 |
| 89298 | 20: 49913380 | 89299 | 20: 49915377 | 89300 | 20: 49918452 | 89323 | 20: 50694458 | 89328 | 20: 50806661 |
| 89338 | 20: 51034291 | 89363 | 20: 51929370 | 89385 | 20: 52736752 | 89394 | 20: 52981985 | 89401 | 20: 53204896 |
| 89405 | 20: 53347849 | 89415 | 20: 53521298 | 89416 | 20: 53526124 | 89420 | 20: 53570726 | 89428 | 20: 53840361 |
| 89442 | 20: 54395462 | 89465 | 20: 55054760 | 89466 | 20: 55055902 | 89478 | 20: 55217698 | 89485 | 20: 55403499 |
| 89500 | 20: 55900412 | 89503 | 20: 55938086 | 89522 | 20: 56361024 | 89526 | 20: 56443806 | 89530 | 20: 56569222 |
| 89532 | 20: 56609894 | 89533 | 20: 56609977 | 89536 | 20: 56692099 | 89541 | 20: 56762906 | 89570 | 20: 57486934 |
| 89571 | 20: 57487439 | 89576 | 20: 57572314 | 89584 | 20: 57778974 | 89595 | 20: 57998700 | 89596 | 20: 57998708 |
| 89597 | 20: 57998777 | 89598 | 20: 57998802 | 89599 | 20: 57998807 | 89600 | 20: 57998808 | 89601 | 20: 57998848 |
| 89602 | 20: 57998909 | 89604 | 20: 58038602 | 89605 | 20: 58038638 | 89606 | 20: 58038676 | 89610 | 20: 58171888 |
| 89622 | 20: 58370776 | 89623 | 20: 58371818 | 89628 | 20: 58425428 | 89630 | 20: 58432597 | 89638 | 20: 58662715 |
| 89652 | 20: 58839976 | 89653 | 20: 58841417 | 89661 | 20: 58985542 | 89665 | 20: 59056598 | 89668 | 20: 59062072 |
| 89675 | 20: 59262668 | 89696 | 20: 59893295 | 89698 | 20: 59918108 | 89699 | 20: 59918158 | 89700 | 20: 59918229 |
| 89702 | 20: 59984000 | 89750 | 20: 61610794 | 89751 | 20: 61610854 | 89755 | 20: 61693804 | 89767 | 20: 61962563 |
| 89773 | 20: 62086784 | 89786 | 20: 62394644 | 89790 | 20: 62513410 | 89792 | 20: 62528844 | 89794 | 20: 62575332 |
| 89799 | 20: 62653294 | 89800 | 20: 62670652 | 89808 | 20: 62848870 | 89810 | 20: 62914219 | 89811 | 20: 62937049 |
| 89812 | 20: 62942539 | 89813 | 20: 62963027 | 89815 | 20: 62971781 | 89828 | 20: 63314880 | 89830 | 20: 63382028 |
| 89831 | 20: 63384182 | 89841 | 20: 63652842 | 89843 | 20: 63711747 | 89848 | 20: 63813002 | 89857 | 20: 63923149 |
| 89858 | 20: 63934159 | 89859 | 20: 63939074 | 89860 | 20: 63939894 | 89861 | 20: 63949508 | 89862 | 20: 63950751 |
| 89863 | 20: 63952037 | 89864 | 20: 63952862 | 89865 | 20: 63954238 | 89866 | 20: 63981759 | 89871 | 20: 64108375 |
| 89891 | 20: 64483792 | 89912 | 20: 65101244 | 89919 | 20: 65253620 | 89927 | 20: 65566194 | 89928 | 20: 65578450 |
| 89929 | 20: 65592431 | 89932 | 20: 65619064 | 89934 | 20: 65691397 | 89936 | 20: 65761375 | 89937 | 20: 65773012 |
| 89938 | 20: 65786774 | 89945 | 20: 65973669 | 89950 | 20: 66089403 | 89955 | 20: 66319547 | 89959 | 20: 66370625 |
| 89960 | 20: 66372293 | 89962 | 20: 66457113 | 89963 | 20: 66464862 | 89964 | 20: 66468697 | 89967 | 20: 66551247 |
| 89968 | 20: 66556459 | 89969 | 20: 66563281 | 89970 | 20: 66563428 | 89971 | 20: 66567783 | 89972 | 20: 66568233 |
| 89973 | 20: 66626549 | 89976 | 20: 66691115 | 89978 | 20: 66696604 | 89979 | 20: 66711575 | 89980 | 20: 66711602 |
| 90001 | 20: 67153888 | 90012 | 20: 67465264 | 90016 | 20: 67521067 | 90026 | 20: 67735557 | 90027 | 20: 67739397 |
| 90028 | 20: 67743005 | 90031 | 20: 67808126 | 90032 | 20: 67819595 | 90035 | 20: 67872456 | 90039 | 20: 67995355 |
| 90041 | 20: 68014834 | 90054 | 20: 68168592 | 90062 | 20: 68323418 | 90063 | 20: 68352541 | 90071 | 20: 68463408 |
| 90072 | 20: 68503575 | 90079 | 20: 68640146 | 90087 | 20: 68746121 | 90095 | 20: 68908716 | 90102 | 20: 68990075 |
| 90129 | 20: 69518490 | 90157 | 20: 70210220 | 90182 | 20: 70941731 | 90183 | 20: 70948562 | 90184 | 20: 70949203 |
| 90185 | 20: 70954723 | 90187 | 20: 70972905 | 90188 | 20: 70973702 | 90189 | 20: 70975538 | 90193 | 20: 71082114 |
| 90194 | 20: 71097489 | 90195 | 20: 71099307 | 90196 | 20: 71104986 | 90197 | 20: 71108601 | 90198 | 20: 71110857 |
| 90199 | 20: 71113969 | 90200 | 20: 71118893 | 90201 | 20: 71119829 | 90202 | 20: 71121420 | 90203 | 20: 71122291 |
| 90204 | 20: 71124183 | 90205 | 20: 71124516 | 90206 | 20: 71127650 | 90207 | 20: 71131787 | 90208 | 20: 71139430 |
| 90209 | 20: 71141395 | 90210 | 20: 71147142 | 90211 | 20: 71147843 | 90212 | 20: 71150904 | 90213 | 20: 71153153 |
| 90214 | 20: 71154456 | 90215 | 20: 71162376 | 90216 | 20: 71172637 | 90217 | 20: 71173872 | 90218 | 20: 71177834 |
| 90219 | 20: 71180101 | 90220 | 20: 71181518 | 90221 | 20: 71183377 | 90222 | 20: 71188295 | 90223 | 20: 71195927 |
| 90224 | 20: 71201866 | 90225 | 20: 71203976 | 90226 | 20: 71206269 | 90227 | 20: 71207699 | 90228 | 20: 71212077 |
| 90229 | 20: 71232016 | 90230 | 20: 71240516 | 90231 | 20: 71290453 | 90232 | 20: 71293773 | 90233 | 20: 71295284 |
| 90234 | 20: 71300630 | 90235 | 20: 71301433 | 90236 | 20: 71313672 | 90237 | 20: 71319974 | 90238 | 20: 71320628 |
| 90239 | 20: 71349322 | 90240 | 20: 71354558 | 90241 | 20: 71355354 | 90242 | 20: 71359918 | 90243 | 20: 71360768 |
| 90244 | 20: 71378946 | 90245 | 20: 71385619 | 90246 | 20: 71386554 | 90247 | 20: 71387739 | 90248 | 20: 71388363 |
| 90249 | 20: 71393864 | 90250 | 20: 71395443 | 90251 | 20: 71396187 | 90252 | 20: 71401124 | 90254 | 20: 71402777 |
| 90255 | 20: 71403559 | 90256 | 20: 71407893 | 90257 | 20: 71417623 | 90258 | 20: 71431631 | 90259 | 20: 71442260 |
| 90260 | 20: 71444098 | 90261 | 20: 71447274 | 90262 | 20: 71448297 | 90263 | 20: 71448849 | 90264 | 20: 71450177 |
| 90265 | 20: 71450861 | 90266 | 20: 71459002 | 90267 | 20: 71472317 | 90268 | 20: 71490172 | 90269 | 20: 71491300 |
| 90272 | 20: 71606988 | 90280 | 20: 71927868 | 90305 | 21: 1606872 | 90320 | 21: 2377105 | 90330 | 21: 2684106 |
| 90336 | 21: 2874100 | 90344 | 21: 3001448 | 90366 | 21: 3866539 | 90375 | 21: 4053839 | 90377 | 21: 4055731 |
| 90378 | 21: 4060826 | 90402 | 21: 4420712 | 90413 | 21: 4948777 | 90420 | 21: 5153009 | 90425 | 21: 5327112 |
| 90428 | 21: 5382657 | 90436 | 21: 5552825 | 90437 | 21: 5575472 | 90441 | 21: 5776117 | 90449 | 21: 5865604 |
| 90453 | 21: 5946488 | 90454 | 21: 5946554 | 90455 | 21: 5961487 | 90460 | 21: 6096270 | 90469 | 21: 6466492 |
| 90471 | 21: 6515336 | 90477 | 21: 6718976 | 90479 | 21: 6821186 | 90480 | 21: 6838984 | 90481 | 21: 6864243 |
| 90488 | 21: 7110184 | 90489 | 21: 7126782 | 90506 | 21: 7582228 | 90508 | 21: 7589812 | 90509 | 21: 7590462 |
| 90520 | 21: 7863440 | 90521 | 21: 7868053 | 90524 | 21: 7907745 | 90530 | 21: 8033587 | 90534 | 21: 8074100 |
| 90535 | 21: 8074643 | 90552 | 21: 8176568 | 90554 | 21: 8190635 | 90555 | 21: 8226423 | 90562 | 21: 8310287 |
| 90604 | 21: 9465633 | 90628 | 21: 9948207 | 90632 | 21: 10010929 | 90642 | 21: 10296960 | 90647 | 21: 10509482 |
| 90653 | 21: 10609946 | 90654 | 21: 10624234 | 90671 | 21: 10957495 | 90679 | 21: 11069313 | 90693 | 21: 11440159 |
| 90694 | 21: 11445014 | 90698 | 21: 11556518 | 90703 | 21: 11654700 | 90743 | 21: 12681133 | 90749 | 21: 12760689 |
| 90754 | 21: 12855624 | 90776 | 21: 13353466 | 90780 | 21: 13514487 | 90794 | 21: 13810003 | 90798 | 21: 13905626 |
| 90805 | 21: 14029521 | 90811 | 21: 14202552 | 90823 | 21: 14517580 | 90824 | 21: 14560006 | 90834 | 21: 14713608 |
| 90840 | 21: 14945241 | 90848 | 21: 15132400 | 90849 | 21: 15138571 | 90851 | 21: 15156831 | 90852 | 21: 15156885 |
| 90853 | 21: 15156911 | 90856 | 21: 15219073 | 90859 | 21: 15326382 | 90888 | 21: 15932209 | 90894 | 21: 16294354 |
| 90896 | 21: 16311821 | 90897 | 21: 16324443 | 90901 | 21: 16376470 | 90905 | 21: 16407620 | 90906 | 21: 16426000 |
| 90908 | 21: 16455704 | 90912 | 21: 16482563 | 90914 | 21: 16507141 | 90919 | 21: 16581532 | 90920 | 21: 16583122 |
| 90922 | 21: 16605546 | 90930 | 21: 16771714 | 90932 | 21: 16775550 | 90933 | 21: 16780485 | 90940 | 21: 16994759 |
| 90945 | 21: 17105820 | 90947 | 21: 17157614 | 90967 | 21: 17696116 | 91010 | 21: 18775346 | 91011 | 21: 18792766 |
| 91012 | 21: 18796836 | 91013 | 21: 18806197 | 91014 | 21: 18807592 | 91015 | 21: 18807654 | 91018 | 21: 18889130 |
| 91019 | 21: 18889216 | 91020 | 21: 18889225 | 91021 | 21: 18897548 | 91022 | 21: 18897617 | 91023 | 21: 18897699 |
| 91024 | 21: 18906129 | 91025 | 21: 18906131 | 91035 | 21: 19086207 | 91044 | 21: 19274749 | 91045 | 21: 19274809 |
| 91049 | 21: 19328532 | 91050 | 21: 19341878 | 91058 | 21: 19448733 | 91059 | 21: 19484240 | 91061 | 21: 19509806 |
| 91065 | 21: 19547342 | 91066 | 21: 19566627 | 91067 | 21: 19571760 | 91068 | 21: 19574086 | 91069 | 21: 19574861 |
| 91071 | 21: 19581384 | 91072 | 21: 19587859 | 91074 | 21: 19591585 | 91077 | 21: 19636076 | 91078 | 21: 19650309 |
| 91079 | 21: 19744871 | 91080 | 21: 19745043 | 91081 | 21: 19765456 | 91082 | 21: 19765736 | 91083 | 21: 19765864 |
| 91084 | 21: 19765895 | 91085 | 21: 19765940 | 91088 | 21: 19791905 | 91089 | 21: 19796807 | 91090 | 21: 19800176 |
| 91092 | 21: 19819891 | 91093 | 21: 19837076 | 91120 | 21: 20576639 | 91123 | 21: 20597014 | 91124 | 21: 20611156 |
| 91129 | 21: 20643657 | 91132 | 21: 20661675 | 91137 | 21: 20707916 | 91139 | 21: 20756163 | 91140 | 21: 20767918 |
| 91148 | 21: 20994233 | 91149 | 21: 20997368 | 91150 | 21: 21002238 | 91153 | 21: 21051410 | 91154 | 21: 21051969 |
| 91155 | 21: 21079825 | 91173 | 21: 21544579 | 91175 | 21: 21547758 | 91176 | 21: 21554110 | 91178 | 21: 21572136 |
| 91189 | 21: 21882815 | 91195 | 21: 21997010 | 91197 | 21: 22018834 | 91201 | 21: 22122091 | 91202 | 21: 22130826 |
| 91203 | 21: 22132465 | 91204 | 21: 22137495 | 91205 | 21: 22138799 | 91208 | 21: 22168604 | 91217 | 21: 22354151 |
| 91222 | 21: 22473177 | 91223 | 21: 22478617 | 91232 | 21: 22695822 | 91234 | 21: 22803320 | 91240 | 21: 22922296 |
| 91244 | 21: 22980503 | 91247 | 21: 23023062 | 91249 | 21: 23081635 | 91250 | 21: 23092163 | 91254 | 21: 23177845 |
| 91255 | 21: 23177853 | 91256 | 21: 23181041 | 91257 | 21: 23190056 | 91258 | 21: 23190653 | 91259 | 21: 23195238 |
| 91260 | 21: 23211467 | 91284 | 21: 24046484 | 91288 | 21: 24273010 | 91293 | 21: 24375735 | 91347 | 21: 26116827 |
| 91361 | 21: 26448325 | 91368 | 21: 26581951 | 91371 | 21: 26637918 | 91383 | 21: 26979057 | 91384 | 21: 26999182 |
| 91385 | 21: 26999251 | 91397 | 21: 27503033 | 91398 | 21: 27508911 | 91407 | 21: 27743344 | 91427 | 21: 28338007 |
| 91441 | 21: 28917960 | 91443 | 21: 28948600 | 91444 | 21: 28961889 | 91448 | 21: 29052667 | 91461 | 21: 29439618 |
| 91465 | 21: 29553919 | 91471 | 21: 29725150 | 91488 | 21: 30205371 | 91489 | 21: 30225427 | 91493 | 21: 30359230 |
| 91494 | 21: 30364775 | 91495 | 21: 30364784 | 91497 | 21: 30380463 | 91498 | 21: 30383546 | 91500 | 21: 30415216 |
| 91504 | 21: 30548247 | 91507 | 21: 30574539 | 91508 | 21: 30583259 | 91512 | 21: 30655015 | 91513 | 21: 30665920 |
| 91514 | 21: 30666267 | 91518 | 21: 30752337 | 91521 | 21: 30836177 | 91525 | 21: 31068352 | 91526 | 21: 31085819 |
| 91528 | 21: 31169053 | 91538 | 21: 31453639 | 91539 | 21: 31464548 | 91540 | 21: 31470928 | 91541 | 21: 31485138 |
| 91542 | 21: 31493718 | 91543 | 21: 31496586 | 91544 | 21: 31499460 | 91545 | 21: 31522156 | 91546 | 21: 31525621 |
| 91547 | 21: 31534000 | 91548 | 21: 31541459 | 91549 | 21: 31545947 | 91550 | 21: 31553803 | 91551 | 21: 31604982 |
| 91553 | 21: 31720553 | 91554 | 21: 31725673 | 91555 | 21: 31734161 | 91556 | 21: 31736971 | 91557 | 21: 31754761 |
| 91558 | 21: 31758189 | 91559 | 21: 31781060 | 91560 | 21: 31791229 | 91563 | 21: 31955982 | 91574 | 21: 32336068 |
| 91575 | 21: 32364556 | 91578 | 21: 32403515 | 91584 | 21: 32692535 | 91585 | 21: 32705474 | 91589 | 21: 32783989 |
| 91590 | 21: 32814280 | 91598 | 21: 33110998 | 91602 | 21: 33166758 | 91603 | 21: 33168203 | 91604 | 21: 33169028 |
| 91605 | 21: 33170204 | 91606 | 21: 33170309 | 91607 | 21: 33171324 | 91608 | 21: 33171538 | 91610 | 21: 33205680 |
| 91611 | 21: 33205893 | 91612 | 21: 33208660 | 91614 | 21: 33215961 | 91615 | 21: 33225284 | 91622 | 21: 33373120 |
| 91623 | 21: 33373124 | 91627 | 21: 33449736 | 91628 | 21: 33458505 | 91629 | 21: 33459344 | 91630 | 21: 33461031 |
| 91631 | 21: 33461192 | 91632 | 21: 33461449 | 91633 | 21: 33465757 | 91634 | 21: 33465758 | 91635 | 21: 33466480 |
| 91636 | 21: 33469426 | 91637 | 21: 33469555 | 91638 | 21: 33469624 | 91639 | 21: 33469729 | 91640 | 21: 33469770 |
| 91641 | 21: 33469982 | 91642 | 21: 33483461 | 91643 | 21: 33487373 | 91645 | 21: 33529381 | 91648 | 21: 33547120 |
| 91650 | 21: 33571261 | 91652 | 21: 33652175 | 91654 | 21: 33661162 | 91670 | 21: 33947582 | 91672 | 21: 33991645 |
| 91675 | 21: 34024711 | 91676 | 21: 34044738 | 91677 | 21: 34049707 | 91680 | 21: 34087450 | 91691 | 21: 34273652 |
| 91692 | 21: 34276064 | 91694 | 21: 34296572 | 91703 | 21: 34434123 | 91705 | 21: 34478879 | 91706 | 21: 34487765 |
| 91707 | 21: 34490950 | 91712 | 21: 34556901 | 91713 | 21: 34556925 | 91714 | 21: 34557109 | 91715 | 21: 34585611 |
| 91718 | 21: 34637208 | 91719 | 21: 34637260 | 91731 | 21: 34938997 | 91732 | 21: 34939108 | 91737 | 21: 35075467 |
| 91741 | 21: 35177228 | 91742 | 21: 35177300 | 91752 | 21: 35387396 | 91753 | 21: 35391211 | 91755 | 21: 35545267 |
| 91761 | 21: 35621906 | 91787 | 21: 36004086 | 91801 | 21: 36349981 | 91807 | 21: 36467140 | 91844 | 21: 37588472 |
| 91855 | 21: 37884949 | 91856 | 21: 37889952 | 91863 | 21: 38352155 | 91866 | 21: 38488688 | 91901 | 21: 39497698 |
| 91902 | 21: 39497796 | 91927 | 21: 40117870 | 91928 | 21: 40123103 | 91930 | 21: 40267167 | 91934 | 21: 40321474 |
| 91950 | 21: 40777585 | 91955 | 21: 41031761 | 91956 | 21: 41063587 | 91958 | 21: 41103681 | 91959 | 21: 41189588 |
| 91960 | 21: 41190356 | 91961 | 21: 41230647 | 91963 | 21: 41268397 | 91968 | 21: 41377730 | 91969 | 21: 41377811 |
| 91971 | 21: 41381520 | 91972 | 21: 41381521 | 91973 | 21: 41381562 | 91979 | 21: 41500659 | 91980 | 21: 41500733 |
| 91988 | 21: 41683205 | 91995 | 21: 41951773 | 91999 | 21: 41985647 | 92001 | 21: 42021205 | 92005 | 21: 42056585 |
| 92006 | 21: 42056665 | 92011 | 21: 42193358 | 92015 | 21: 42263974 | 92025 | 21: 42613095 | 92026 | 21: 42613704 |
| 92027 | 21: 42615428 | 92028 | 21: 42632006 | 92036 | 21: 42789940 | 92037 | 21: 42789959 | 92038 | 21: 42794024 |
| 92042 | 21: 42894892 | 92043 | 21: 42894971 | 92044 | 21: 42906559 | 92048 | 21: 42991321 | 92060 | 21: 43369442 |
| 92073 | 21: 43694807 | 92090 | 21: 44134802 | 92093 | 21: 44170697 | 92094 | 21: 44193483 | 92095 | 21: 44198391 |
| 92096 | 21: 44204274 | 92097 | 21: 44204999 | 92098 | 21: 44208959 | 92103 | 21: 44374543 | 92108 | 21: 44464711 |
| 92111 | 21: 44510750 | 92112 | 21: 44558742 | 92119 | 21: 44670112 | 92124 | 21: 44711056 | 92125 | 21: 44711119 |
| 92128 | 21: 44747742 | 92131 | 21: 44794407 | 92137 | 21: 44874950 | 92146 | 21: 45364186 | 92153 | 21: 45617906 |
| 92162 | 21: 45840926 | 92168 | 21: 46169637 | 92177 | 21: 46371720 | 92178 | 21: 46372978 | 92179 | 21: 46375756 |
| 92180 | 21: 46377528 | 92181 | 21: 46378483 | 92182 | 21: 46399567 | 92183 | 21: 46411679 | 92207 | 21: 47030345 |
| 92209 | 21: 47120773 | 92210 | 21: 47135961 | 92235 | 21: 47300254 | 92252 | 21: 47315681 | 92255 | 21: 47399940 |
| 92260 | 21: 47464301 | 92272 | 21: 47936288 | 92293 | 21: 48482014 | 92308 | 21: 48897167 | 92315 | 21: 49088678 |
| 92316 | 21: 49165291 | 92317 | 21: 49165372 | 92325 | 21: 49212043 | 92348 | 21: 49756597 | 92349 | 21: 49773549 |
| 92414 | 21: 51757967 | 92423 | 21: 52002988 | 92430 | 21: 52140964 | 92438 | 21: 52391191 | 92447 | 21: 52590908 |
| 92459 | 21: 53100162 | 92467 | 21: 53274187 | 92468 | 21: 53295524 | 92469 | 21: 53295551 | 92473 | 21: 53382594 |
| 92494 | 21: 53955771 | 92511 | 21: 54486141 | 92519 | 21: 54707788 | 92533 | 21: 54895636 | 92534 | 21: 54906059 |
| 92535 | 21: 54910848 | 92536 | 21: 54911588 | 92537 | 21: 54911598 | 92538 | 21: 54919544 | 92540 | 21: 54956256 |
| 92544 | 21: 55027058 | 92549 | 21: 55139938 | 92551 | 21: 55157787 | 92553 | 21: 55268142 | 92555 | 21: 55276126 |
| 92559 | 21: 55336100 | 92567 | 21: 55534889 | 92600 | 21: 56030046 | 92609 | 21: 56102239 | 92611 | 21: 56151393 |
| 92612 | 21: 56165597 | 92613 | 21: 56174882 | 92614 | 21: 56184939 | 92616 | 21: 56194199 | 92617 | 21: 56195714 |
| 92618 | 21: 56211969 | 92625 | 21: 56552387 | 92627 | 21: 56570919 | 92637 | 21: 56804066 | 92689 | 21: 57070861 |
| 92690 | 21: 57070865 | 92713 | 21: 57163461 | 92714 | 21: 57163519 | 92730 | 21: 57357698 | 92737 | 21: 57514823 |
| 92740 | 21: 57650013 | 92744 | 21: 57789826 | 92745 | 21: 57815184 | 92746 | 21: 57841247 | 92752 | 21: 58086488 |
| 92760 | 21: 58328406 | 92761 | 21: 58335169 | 92762 | 21: 58348854 | 92765 | 21: 58457450 | 92769 | 21: 58542608 |
| 92777 | 21: 58649278 | 92778 | 21: 58649996 | 92779 | 21: 58650822 | 92780 | 21: 58651655 | 92781 | 21: 58651789 |
| 92782 | 21: 58653170 | 92783 | 21: 58653759 | 92784 | 21: 58653796 | 92785 | 21: 58654257 | 92786 | 21: 58657343 |
| 92787 | 21: 58730952 | 92792 | 21: 58844584 | 92794 | 21: 58897764 | 92812 | 21: 59280395 | 92813 | 21: 59290376 |
| 92816 | 21: 59410654 | 92817 | 21: 59450928 | 92821 | 21: 59494769 | 92838 | 21: 59798491 | 92846 | 21: 59885566 |
| 92847 | 21: 59892560 | 92854 | 21: 59967817 | 92857 | 21: 59995456 | 92858 | 21: 60034051 | 92860 | 21: 60070408 |
| 92864 | 21: 60128571 | 92867 | 21: 60147487 | 92870 | 21: 60222745 | 92872 | 21: 60258085 | 92875 | 21: 60285746 |
| 92878 | 21: 60309341 | 92879 | 21: 60318597 | 92880 | 21: 60364407 | 92887 | 21: 60531623 | 92888 | 21: 60531761 |
| 92914 | 21: 60963159 | 92929 | 21: 61332759 | 92935 | 21: 61617246 | 92936 | 21: 61637616 | 92937 | 21: 61639799 |
| 92938 | 21: 61640498 | 92957 | 21: 62025836 | 92973 | 21: 62390233 | 93001 | 21: 63134292 | 93003 | 21: 63222235 |
| 93004 | 21: 63258242 | 93010 | 21: 63480195 | 93017 | 21: 63603801 | 93024 | 21: 63831078 | 93029 | 21: 63935578 |
| 93046 | 21: 64333571 | 93050 | 21: 64428779 | 93051 | 21: 64450577 | 93052 | 21: 64452975 | 93065 | 21: 64633547 |
| 93070 | 21: 64699689 | 93077 | 21: 64841812 | 93078 | 21: 64841843 | 93079 | 21: 64841865 | 93080 | 21: 64850271 |
| 93084 | 21: 64922535 | 93086 | 21: 64988072 | 93088 | 21: 65014550 | 93089 | 21: 65027672 | 93090 | 21: 65029870 |
| 93091 | 21: 65051618 | 93092 | 21: 65052179 | 93093 | 21: 65052717 | 93094 | 21: 65054313 | 93095 | 21: 65055762 |
| 93096 | 21: 65056739 | 93097 | 21: 65060441 | 93098 | 21: 65061116 | 93099 | 21: 65067323 | 93100 | 21: 65073410 |
| 93101 | 21: 65073892 | 93102 | 21: 65075325 | 93103 | 21: 65079623 | 93104 | 21: 65080143 | 93105 | 21: 65082481 |
| 93114 | 21: 65232598 | 93167 | 21: 66311776 | 93183 | 21: 66723461 | 93189 | 21: 66870945 | 93190 | 21: 66929334 |
| 93191 | 21: 66932880 | 93192 | 21: 66934300 | 93193 | 21: 66936241 | 93194 | 21: 66938188 | 93195 | 21: 66942278 |
| 93197 | 21: 66945357 | 93204 | 21: 67094952 | 93208 | 21: 67149363 | 93215 | 21: 67449798 | 93221 | 21: 67552612 |
| 93231 | 21: 67865058 | 93232 | 21: 67876373 | 93240 | 21: 67965438 | 93251 | 21: 68196501 | 93252 | 21: 68202138 |
| 93253 | 21: 68202185 | 93255 | 21: 68230479 | 93258 | 21: 68286931 | 93260 | 21: 68335052 | 93268 | 21: 68506930 |
| 93270 | 21: 68519125 | 93271 | 21: 68522170 | 93272 | 21: 68529965 | 93273 | 21: 68530561 | 93274 | 21: 68530572 |
| 93276 | 21: 68537086 | 93280 | 21: 68668451 | 93294 | 21: 69004265 | 93301 | 21: 69165607 | 93304 | 21: 69229046 |
| 93308 | 21: 69352283 | 93309 | 21: 69352557 | 93310 | 21: 69353990 | 93312 | 21: 69409067 | 93314 | 21: 69440442 |
| 93315 | 21: 69440464 | 93316 | 21: 69440466 | 93317 | 21: 69442039 | 93320 | 21: 69557781 | 93321 | 21: 69574694 |
| 93322 | 21: 69582716 | 93324 | 21: 69629308 | 93325 | 21: 69642755 | 93326 | 21: 69660286 | 93331 | 21: 69725960 |
| 93332 | 21: 69764707 | 93333 | 21: 69765916 | 93334 | 21: 69766081 | 93335 | 21: 69770916 | 93345 | 22: 378758 |
| 93353 | 22: 651171 | 93363 | 22: 912338 | 93366 | 22: 940171 | 93367 | 22: 940370 | 93395 | 22: 1560877 |
| 93401 | 22: 1738773 | 93402 | 22: 1738804 | 93403 | 22: 1738805 | 93404 | 22: 1746536 | 93414 | 22: 1927654 |
| 93432 | 22: 2329624 | 93449 | 22: 2751520 | 93450 | 22: 2752726 | 93456 | 22: 2877258 | 93457 | 22: 2892709 |
| 93465 | 22: 3056974 | 93471 | 22: 3197001 | 93473 | 22: 3220010 | 93475 | 22: 3269948 | 93482 | 22: 3359434 |
| 93493 | 22: 3596980 | 93499 | 22: 3798423 | 93501 | 22: 3834731 | 93504 | 22: 3914101 | 93506 | 22: 3960951 |
| 93529 | 22: 4497784 | 93540 | 22: 4729907 | 93546 | 22: 4842711 | 93551 | 22: 4922676 | 93556 | 22: 5030837 |
| 93559 | 22: 5120220 | 93561 | 22: 5160439 | 93562 | 22: 5165375 | 93564 | 22: 5179197 | 93575 | 22: 5341524 |
| 93576 | 22: 5345473 | 93577 | 22: 5391435 | 93584 | 22: 5485152 | 93595 | 22: 5647094 | 93597 | 22: 5693798 |
| 93598 | 22: 5721636 | 93600 | 22: 5764445 | 93605 | 22: 5883723 | 93612 | 22: 5950788 | 93616 | 22: 6008417 |
| 93617 | 22: 6017167 | 93618 | 22: 6052161 | 93622 | 22: 6199633 | 93627 | 22: 6252438 | 93630 | 22: 6293377 |
| 93636 | 22: 6370667 | 93643 | 22: 6488129 | 93645 | 22: 6498701 | 93657 | 22: 6784495 | 93660 | 22: 6839952 |
| 93671 | 22: 6996393 | 93685 | 22: 7428239 | 93686 | 22: 7434501 | 93697 | 22: 7651542 | 93702 | 22: 7841139 |
| 93703 | 22: 7908696 | 93723 | 22: 8411667 | 93735 | 22: 8761701 | 93772 | 22: 9753217 | 93774 | 22: 9795347 |
| 93783 | 22: 10006268 | 93784 | 22: 10006273 | 93785 | 22: 10006376 | 93787 | 22: 10035911 | 93789 | 22: 10081106 |
| 93790 | 22: 10081118 | 93791 | 22: 10081165 | 93792 | 22: 10081187 | 93793 | 22: 10081208 | 93794 | 22: 10081215 |
| 93795 | 22: 10081251 | 93796 | 22: 10081295 | 93801 | 22: 10172170 | 93804 | 22: 10236047 | 93809 | 22: 10290595 |
| 93810 | 22: 10345715 | 93812 | 22: 10401777 | 93836 | 22: 11003544 | 93841 | 22: 11122201 | 93850 | 22: 11391522 |
| 93858 | 22: 11578648 | 93860 | 22: 11718954 | 93861 | 22: 11720712 | 93865 | 22: 11758613 | 93867 | 22: 11831403 |
| 93868 | 22: 11832624 | 93869 | 22: 11889790 | 93870 | 22: 11889853 | 93871 | 22: 11890006 | 93872 | 22: 11890127 |
| 93878 | 22: 11973585 | 93879 | 22: 11977784 | 93880 | 22: 11977859 | 93881 | 22: 11978609 | 93882 | 22: 11978855 |
| 93883 | 22: 11978903 | 93886 | 22: 12019884 | 93892 | 22: 12195260 | 93897 | 22: 12303365 | 93900 | 22: 12377148 |
| 93901 | 22: 12430483 | 93902 | 22: 12436371 | 93904 | 22: 12451350 | 93907 | 22: 12503200 | 93908 | 22: 12506458 |
| 93917 | 22: 12662972 | 93921 | 22: 12724433 | 93930 | 22: 12923416 | 93936 | 22: 13091338 | 93943 | 22: 13359849 |
| 93951 | 22: 13559915 | 93952 | 22: 13582865 | 93969 | 22: 14142677 | 93970 | 22: 14145371 | 93973 | 22: 14181865 |
| 93976 | 22: 14213464 | 93978 | 22: 14312375 | 93984 | 22: 14511531 | 93985 | 22: 14512264 | 93986 | 22: 14516780 |
| 93987 | 22: 14516787 | 93988 | 22: 14518692 | 93989 | 22: 14519180 | 94006 | 22: 15072933 | 94008 | 22: 15123459 |
| 94019 | 22: 15471657 | 94022 | 22: 15504111 | 94027 | 22: 15605731 | 94051 | 22: 16322403 | 94052 | 22: 16322451 |
| 94053 | 22: 16322488 | 94054 | 22: 16353524 | 94062 | 22: 16546381 | 94063 | 22: 16548801 | 94064 | 22: 16561111 |
| 94065 | 22: 16567113 | 94066 | 22: 16567586 | 94067 | 22: 16568933 | 94073 | 22: 16666585 | 94077 | 22: 16725586 |
| 94078 | 22: 16728120 | 94080 | 22: 16741450 | 94082 | 22: 16757417 | 94083 | 22: 16773003 | 94084 | 22: 16802200 |
| 94085 | 22: 16804567 | 94100 | 22: 17418863 | 94113 | 22: 17718315 | 94116 | 22: 17791469 | 94123 | 22: 17937879 |
| 94125 | 22: 17953330 | 94130 | 22: 18035176 | 94132 | 22: 18061536 | 94151 | 22: 18541264 | 94156 | 22: 18614346 |
| 94160 | 22: 18696586 | 94164 | 22: 18832117 | 94175 | 22: 19093982 | 94177 | 22: 19212118 | 94185 | 22: 19376422 |
| 94215 | 22: 20299573 | 94221 | 22: 20368774 | 94234 | 22: 20830719 | 94259 | 22: 21264256 | 94260 | 22: 21281505 |
| 94262 | 22: 21343700 | 94270 | 22: 21553615 | 94272 | 22: 21581948 | 94275 | 22: 21638280 | 94282 | 22: 21687964 |
| 94284 | 22: 21691311 | 94285 | 22: 21691369 | 94286 | 22: 21696971 | 94287 | 22: 21697320 | 94289 | 22: 21780680 |
| 94295 | 22: 21936689 | 94298 | 22: 22018100 | 94301 | 22: 22096896 | 94302 | 22: 22130081 | 94304 | 22: 22207036 |
| 94305 | 22: 22280513 | 94306 | 22: 22280554 | 94312 | 22: 22511637 | 94321 | 22: 22847965 | 94325 | 22: 22910885 |
| 94328 | 22: 22978479 | 94390 | 22: 24919962 | 94419 | 22: 25887179 | 94426 | 22: 26004558 | 94427 | 22: 26004600 |
| 94428 | 22: 26006402 | 94429 | 22: 26028973 | 94431 | 22: 26048198 | 94432 | 22: 26048256 | 94433 | 22: 26055574 |
| 94437 | 22: 26092272 | 94438 | 22: 26092293 | 94442 | 22: 26179382 | 94443 | 22: 26199906 | 94446 | 22: 26342475 |
| 94474 | 22: 27220605 | 94480 | 22: 27400091 | 94490 | 22: 27635934 | 94507 | 22: 28135962 | 94512 | 22: 28188835 |
| 94514 | 22: 28211359 | 94518 | 22: 28297855 | 94523 | 22: 28404142 | 94524 | 22: 28404219 | 94529 | 22: 28481900 |
| 94531 | 22: 28515749 | 94533 | 22: 28587264 | 94534 | 22: 28588309 | 94539 | 22: 28733810 | 94541 | 22: 28784852 |
| 94542 | 22: 28784985 | 94543 | 22: 28792168 | 94545 | 22: 28835841 | 94546 | 22: 28835907 | 94547 | 22: 28835973 |
| 94548 | 22: 28840124 | 94549 | 22: 28840129 | 94550 | 22: 28843084 | 94551 | 22: 28844382 | 94552 | 22: 28845975 |
| 94554 | 22: 28862947 | 94555 | 22: 28938955 | 94557 | 22: 29005992 | 94566 | 22: 29299265 | 94581 | 22: 29705813 |
| 94582 | 22: 29709388 | 94583 | 22: 29712615 | 94604 | 22: 30274003 | 94607 | 22: 30417109 | 94608 | 22: 30436771 |
| 94610 | 22: 30515471 | 94624 | 22: 30808716 | 94638 | 22: 31133292 | 94639 | 22: 31135175 | 94640 | 22: 31141034 |
| 94661 | 22: 31835374 | 94662 | 22: 31861512 | 94663 | 22: 31891766 | 94666 | 22: 32022974 | 94673 | 22: 32156119 |
| 94674 | 22: 32186880 | 94675 | 22: 32233824 | 94676 | 22: 32246231 | 94679 | 22: 32399020 | 94685 | 22: 32572065 |
| 94688 | 22: 32679434 | 94698 | 22: 32982493 | 94700 | 22: 32993742 | 94701 | 22: 33017003 | 94702 | 22: 33053278 |
| 94708 | 22: 33142515 | 94728 | 22: 33935530 | 94729 | 22: 33944757 | 94733 | 22: 33983138 | 94768 | 22: 34968813 |
| 94771 | 22: 35012254 | 94774 | 22: 35039773 | 94781 | 22: 35173959 | 94791 | 22: 35329495 | 94793 | 22: 35423874 |
| 94796 | 22: 35459751 | 94797 | 22: 35460784 | 94799 | 22: 35523854 | 94810 | 22: 35736939 | 94812 | 22: 35774994 |
| 94815 | 22: 35862159 | 94816 | 22: 35890192 | 94817 | 22: 35901222 | 94826 | 22: 36031535 | 94837 | 22: 36381197 |
| 94844 | 22: 36496828 | 94846 | 22: 36551821 | 94848 | 22: 36609940 | 94856 | 22: 36967669 | 94865 | 22: 37191405 |
| 94866 | 22: 37210076 | 94872 | 22: 37384626 | 94873 | 22: 37391008 | 94876 | 22: 37473284 | 94883 | 22: 37617112 |
| 94884 | 22: 37620082 | 94885 | 22: 37622201 | 94886 | 22: 37624592 | 94887 | 22: 37627226 | 94888 | 22: 37628993 |
| 94889 | 22: 37635996 | 94890 | 22: 37636848 | 94891 | 22: 37637997 | 94892 | 22: 37640217 | 94893 | 22: 37653222 |
| 94917 | 22: 38367562 | 94919 | 22: 38384223 | 94924 | 22: 38493420 | 94928 | 22: 38548453 | 94934 | 22: 38617876 |
| 94944 | 22: 38801073 | 94955 | 22: 39102590 | 94963 | 22: 39227562 | 94964 | 22: 39234430 | 94965 | 22: 39240885 |
| 94977 | 22: 39399375 | 94978 | 22: 39399383 | 94996 | 22: 39664313 | 95001 | 22: 39736141 | 95002 | 22: 39736166 |
| 95003 | 22: 39742270 | 95005 | 22: 39764001 | 95007 | 22: 39806230 | 95022 | 22: 40159960 | 95028 | 22: 40412439 |
| 95030 | 22: 40474842 | 95031 | 22: 40497320 | 95033 | 22: 40497948 | 95036 | 22: 40553964 | 95043 | 22: 40698664 |
| 95044 | 22: 40699122 | 95046 | 22: 40709407 | 95064 | 22: 41331703 | 95077 | 22: 41673525 | 95078 | 22: 41697991 |
| 95104 | 22: 42421167 | 95108 | 22: 42552768 | 95118 | 22: 42753833 | 95120 | 22: 42860068 | 95131 | 22: 43070171 |
| 95133 | 22: 43103372 | 95135 | 22: 43152197 | 95137 | 22: 43171695 | 95139 | 22: 43185881 | 95142 | 22: 43216398 |
| 95145 | 22: 43241133 | 95148 | 22: 43280264 | 95151 | 22: 43327456 | 95152 | 22: 43328177 | 95158 | 22: 43532708 |
| 95162 | 22: 43617671 | 95170 | 22: 43831970 | 95172 | 22: 43860815 | 95190 | 22: 44240079 | 95191 | 22: 44242443 |
| 95195 | 22: 44303591 | 95212 | 22: 44704423 | 95217 | 22: 44800260 | 95225 | 22: 45101725 | 95232 | 22: 45343274 |
| 95245 | 22: 45656061 | 95247 | 22: 45691255 | 95252 | 22: 45783940 | 95255 | 22: 45826883 | 95270 | 22: 46040006 |
| 95273 | 22: 46071530 | 95291 | 22: 46619809 | 95296 | 22: 46740639 | 95298 | 22: 46748886 | 95311 | 22: 46981263 |
| 95312 | 22: 46981314 | 95315 | 22: 47033448 | 95318 | 22: 47057561 | 95331 | 22: 47235742 | 95333 | 22: 47261646 |
| 95337 | 22: 47402038 | 95342 | 22: 47467741 | 95349 | 22: 47617100 | 95352 | 22: 47677699 | 95356 | 22: 47817718 |
| 95361 | 22: 48000435 | 95364 | 22: 48019220 | 95369 | 22: 48066613 | 95370 | 22: 48091175 | 95371 | 22: 48107831 |
| 95372 | 22: 48170389 | 95373 | 22: 48171831 | 95380 | 22: 48435818 | 95381 | 22: 48449692 | 95386 | 22: 48687235 |
| 95388 | 22: 48709706 | 95391 | 22: 48761536 | 95395 | 22: 48961009 | 95396 | 22: 48979936 | 95397 | 22: 48988156 |
| 95398 | 22: 49025901 | 95399 | 22: 49040313 | 95400 | 22: 49041656 | 95405 | 22: 49152003 | 95407 | 22: 49218903 |
| 95411 | 22: 49396397 | 95412 | 22: 49411257 | 95413 | 22: 49416701 | 95417 | 22: 49475649 | 95422 | 22: 49751795 |
| 95427 | 22: 49845416 | 95428 | 22: 49855390 | 95429 | 22: 49862171 | 95430 | 22: 49874759 | 95437 | 22: 50031711 |
| 95443 | 22: 50110984 | 95444 | 22: 50127865 | 95446 | 22: 50174136 | 95448 | 22: 50267951 | 95453 | 22: 50398849 |
| 95463 | 22: 50615575 | 95464 | 22: 50626022 | 95467 | 22: 50669330 | 95468 | 22: 50669331 | 95471 | 22: 50732557 |
| 95472 | 22: 50752636 | 95475 | 22: 50849444 | 95488 | 22: 51284290 | 95490 | 22: 51317516 | 95495 | 22: 51400864 |
| 95502 | 22: 51573666 | 95509 | 22: 51800322 | 95517 | 22: 51985899 | 95518 | 22: 51994258 | 95521 | 22: 52090420 |
| 95522 | 22: 52090421 | 95523 | 22: 52090571 | 95526 | 22: 52113515 | 95528 | 22: 52142954 | 95535 | 22: 52398536 |
| 95536 | 22: 52409661 | 95545 | 22: 52642109 | 95547 | 22: 52689484 | 95555 | 22: 52876456 | 95559 | 22: 52889478 |
| 95562 | 22: 52940887 | 95567 | 22: 52998347 | 95568 | 22: 52998797 | 95569 | 22: 52998800 | 95570 | 22: 52999211 |
| 95571 | 22: 52999397 | 95573 | 22: 53017868 | 95586 | 22: 53333639 | 95595 | 22: 53587524 | 95596 | 22: 53630193 |
| 95597 | 22: 53632109 | 95599 | 22: 53667747 | 95601 | 22: 53701852 | 95603 | 22: 53750298 | 95604 | 22: 53771417 |
| 95608 | 22: 53814947 | 95609 | 22: 53815430 | 95610 | 22: 53823185 | 95617 | 22: 53952061 | 95625 | 22: 54050342 |
| 95626 | 22: 54084951 | 95634 | 22: 54271229 | 95636 | 22: 54325220 | 95637 | 22: 54325294 | 95638 | 22: 54326413 |
| 95639 | 22: 54326417 | 95640 | 22: 54326525 | 95652 | 22: 54582223 | 95654 | 22: 54586616 | 95655 | 22: 54590257 |
| 95656 | 22: 54591674 | 95657 | 22: 54601062 | 95659 | 22: 54619999 | 95660 | 22: 54656963 | 95661 | 22: 54657085 |
| 95681 | 22: 55222425 | 95682 | 22: 55249217 | 95684 | 22: 55290303 | 95685 | 22: 55290330 | 95687 | 22: 55290410 |
| 95688 | 22: 55290479 | 95689 | 22: 55290914 | 95690 | 22: 55291002 | 95691 | 22: 55291168 | 95692 | 22: 55291236 |
| 95693 | 22: 55291381 | 95694 | 22: 55291382 | 95697 | 22: 55338384 | 95699 | 22: 55379431 | 95700 | 22: 55413027 |
| 95705 | 22: 55446332 | 95706 | 22: 55455982 | 95709 | 22: 55483225 | 95713 | 22: 55579849 | 95725 | 22: 55804900 |
| 95728 | 22: 55847268 | 95733 | 22: 55887174 | 95734 | 22: 55887346 | 95735 | 22: 55887394 | 95736 | 22: 55887397 |
| 95737 | 22: 55887398 | 95738 | 22: 55887409 | 95739 | 22: 55887425 | 95740 | 22: 55887575 | 95741 | 22: 55888374 |
| 95746 | 22: 55952909 | 95759 | 22: 56301696 | 95761 | 22: 56325207 | 95762 | 22: 56325210 | 95763 | 22: 56325225 |
| 95764 | 22: 56325235 | 95765 | 22: 56325272 | 95766 | 22: 56325600 | 95767 | 22: 56325606 | 95769 | 22: 56325745 |
| 95770 | 22: 56325754 | 95771 | 22: 56326527 | 95772 | 22: 56329687 | 95773 | 22: 56329721 | 95774 | 22: 56332166 |
| 95775 | 22: 56340185 | 95777 | 22: 56377918 | 95778 | 22: 56388281 | 95780 | 22: 56396437 | 95791 | 22: 56484402 |
| 95792 | 22: 56513273 | 95797 | 22: 56546272 | 95800 | 22: 56616418 | 95801 | 22: 56632616 | 95822 | 22: 57017818 |
| 95823 | 22: 57017940 | 95825 | 22: 57074332 | 95827 | 22: 57132556 | 95830 | 22: 57180726 | 95831 | 22: 57198542 |
| 95832 | 22: 57228439 | 95833 | 22: 57257734 | 95834 | 22: 57268915 | 95840 | 22: 57427960 | 95841 | 22: 57434193 |
| 95843 | 22: 57453058 | 95846 | 22: 57478438 | 95847 | 22: 57511375 | 95851 | 22: 57532676 | 95855 | 22: 57579073 |
| 95857 | 22: 57629519 | 95858 | 22: 57638240 | 95869 | 22: 57759457 | 95870 | 22: 57764469 | 95871 | 22: 57766664 |
| 95872 | 22: 57792445 | 95890 | 22: 58069483 | 95891 | 22: 58075933 | 95892 | 22: 58086195 | 95905 | 22: 58427446 |
| 95907 | 22: 58446882 | 95911 | 22: 58506677 | 95925 | 22: 58804900 | 95938 | 22: 59105276 | 95939 | 22: 59111787 |
| 95945 | 22: 59165452 | 95947 | 22: 59174986 | 95950 | 22: 59237930 | 95961 | 22: 59518872 | 95966 | 22: 59625926 |
| 95978 | 22: 59729840 | 95982 | 22: 59783092 | 95983 | 22: 59831717 | 95989 | 22: 59928044 | 96005 | 22: 60263301 |
| 96007 | 22: 60314302 | 96008 | 22: 60344480 | 96009 | 22: 60369311 | 96013 | 22: 60433161 | 96014 | 22: 60450289 |
| 96015 | 22: 60450822 | 96016 | 22: 60450826 | 96021 | 22: 60583428 | 96024 | 22: 60664148 | 96028 | 23: 98715 |
| 96048 | 23: 908722 | 96059 | 23: 1233960 | 96067 | 23: 1413088 | 96081 | 23: 1806494 | 96089 | 23: 2027290 |
| 96091 | 23: 2085668 | 96102 | 23: 2295458 | 96103 | 23: 2327306 | 96109 | 23: 2490639 | 96115 | 23: 2633185 |
| 96119 | 23: 2653923 | 96121 | 23: 2672734 | 96122 | 23: 2681218 | 96131 | 23: 2932961 | 96132 | 23: 2932963 |
| 96133 | 23: 2933026 | 96134 | 23: 2933089 | 96135 | 23: 2933119 | 96136 | 23: 2943350 | 96137 | 23: 2944259 |
| 96138 | 23: 2945377 | 96147 | 23: 3182108 | 96150 | 23: 3278552 | 96152 | 23: 3363897 | 96154 | 23: 3397926 |
| 96155 | 23: 3401808 | 96156 | 23: 3422753 | 96157 | 23: 3441692 | 96158 | 23: 3451376 | 96167 | 23: 3623220 |
| 96169 | 23: 3649638 | 96172 | 23: 3661124 | 96173 | 23: 3691486 | 96174 | 23: 3694848 | 96187 | 23: 3953291 |
| 96195 | 23: 4055037 | 96207 | 23: 4270316 | 96211 | 23: 4397147 | 96257 | 23: 5221777 | 96270 | 23: 5551234 |
| 96273 | 23: 5596536 | 96274 | 23: 5598882 | 96275 | 23: 5598939 | 96276 | 23: 5598954 | 96277 | 23: 5598967 |
| 96291 | 23: 6007499 | 96302 | 23: 6313109 | 96315 | 23: 6596002 | 96316 | 23: 6596059 | 96319 | 23: 6615760 |
| 96320 | 23: 6619709 | 96321 | 23: 6619777 | 96334 | 23: 6834687 | 96343 | 23: 7086052 | 96348 | 23: 7176749 |
| 96349 | 23: 7185030 | 96351 | 23: 7220926 | 96354 | 23: 7287024 | 96362 | 23: 7470856 | 96366 | 23: 7588787 |
| 96367 | 23: 7590703 | 96368 | 23: 7604788 | 96369 | 23: 7627114 | 96370 | 23: 7627927 | 96385 | 23: 8019039 |
| 96386 | 23: 8021311 | 96388 | 23: 8043634 | 96394 | 23: 8257417 | 96395 | 23: 8265499 | 96402 | 23: 8382452 |
| 96403 | 23: 8386569 | 96405 | 23: 8392718 | 96406 | 23: 8394550 | 96407 | 23: 8403739 | 96409 | 23: 8430336 |
| 96410 | 23: 8437470 | 96411 | 23: 8444830 | 96412 | 23: 8458254 | 96423 | 23: 8693541 | 96428 | 23: 8861332 |
| 96429 | 23: 8861347 | 96435 | 23: 8977884 | 96438 | 23: 9024159 | 96439 | 23: 9024206 | 96440 | 23: 9024246 |
| 96447 | 23: 9163290 | 96448 | 23: 9164368 | 96452 | 23: 9341201 | 96457 | 23: 9496576 | 96460 | 23: 9564042 |
| 96461 | 23: 9565542 | 96462 | 23: 9583880 | 96464 | 23: 9685892 | 96466 | 23: 9745811 | 96467 | 23: 9767204 |
| 96476 | 23: 10029783 | 96483 | 23: 10140851 | 96485 | 23: 10256230 | 96486 | 23: 10257763 | 96487 | 23: 10257827 |
| 96488 | 23: 10258971 | 96489 | 23: 10259318 | 96490 | 23: 10259504 | 96492 | 23: 10261010 | 96493 | 23: 10262012 |
| 96494 | 23: 10262085 | 96495 | 23: 10262281 | 96496 | 23: 10262341 | 96497 | 23: 10262386 | 96498 | 23: 10262523 |
| 96501 | 23: 10328877 | 96506 | 23: 10459370 | 96514 | 23: 10615240 | 96515 | 23: 10638380 | 96519 | 23: 10710493 |
| 96523 | 23: 10803832 | 96524 | 23: 10870495 | 96527 | 23: 10883412 | 96528 | 23: 10883649 | 96529 | 23: 10890338 |
| 96530 | 23: 10913209 | 96532 | 23: 10921995 | 96533 | 23: 10922014 | 96538 | 23: 11106125 | 96554 | 23: 11574894 |
| 96555 | 23: 11592749 | 96556 | 23: 11599745 | 96564 | 23: 11737588 | 96566 | 23: 11785803 | 96573 | 23: 11925495 |
| 96575 | 23: 11950761 | 96576 | 23: 11956619 | 96586 | 23: 12073751 | 96591 | 23: 12208427 | 96594 | 23: 12235079 |
| 96603 | 23: 12561054 | 96604 | 23: 12561114 | 96605 | 23: 12561116 | 96606 | 23: 12561252 | 96607 | 23: 12561302 |
| 96608 | 23: 12564591 | 96609 | 23: 12566537 | 96625 | 23: 12975952 | 96626 | 23: 13002407 | 96631 | 23: 13062813 |
| 96632 | 23: 13062870 | 96633 | 23: 13063271 | 96634 | 23: 13065594 | 96635 | 23: 13065660 | 96636 | 23: 13066259 |
| 96637 | 23: 13073019 | 96638 | 23: 13083039 | 96639 | 23: 13096550 | 96640 | 23: 13119734 | 96645 | 23: 13155594 |
| 96650 | 23: 13360736 | 96668 | 23: 13797670 | 96669 | 23: 13803074 | 96673 | 23: 13844075 | 96682 | 23: 13890619 |
| 96683 | 23: 13913723 | 96684 | 23: 13917180 | 96711 | 23: 14503938 | 96713 | 23: 14550205 | 96726 | 23: 14801418 |
| 96736 | 23: 14938004 | 96740 | 23: 14977766 | 96744 | 23: 15085370 | 96746 | 23: 15116016 | 96747 | 23: 15116055 |
| 96748 | 23: 15118596 | 96749 | 23: 15119248 | 96750 | 23: 15119542 | 96751 | 23: 15119571 | 96752 | 23: 15119572 |
| 96753 | 23: 15119619 | 96754 | 23: 15135050 | 96761 | 23: 15271919 | 96763 | 23: 15283396 | 96766 | 23: 15345255 |
| 96773 | 23: 15533652 | 96778 | 23: 15581761 | 96795 | 23: 15759615 | 96801 | 23: 15883997 | 96803 | 23: 15932643 |
| 96813 | 23: 16272106 | 96820 | 23: 16456191 | 96822 | 23: 16466008 | 96823 | 23: 16479564 | 96829 | 23: 16661447 |
| 96831 | 23: 16686014 | 96832 | 23: 16714066 | 96835 | 23: 16792993 | 96836 | 23: 16801536 | 96843 | 23: 17062554 |
| 96859 | 23: 17556957 | 96868 | 23: 17708671 | 96869 | 23: 17718607 | 96870 | 23: 17737639 | 96890 | 23: 18161337 |
| 96891 | 23: 18200714 | 96894 | 23: 18216510 | 96935 | 23: 19331450 | 96936 | 23: 19366522 | 96939 | 23: 19415329 |
| 96966 | 23: 19751742 | 96970 | 23: 19767163 | 96974 | 23: 19826206 | 96976 | 23: 19925315 | 96982 | 23: 19970969 |
| 96985 | 23: 20042631 | 96986 | 23: 20052143 | 96989 | 23: 20102219 | 96991 | 23: 20121033 | 96997 | 23: 20203703 |
| 96998 | 23: 20223241 | 97007 | 23: 20409619 | 97008 | 23: 20409671 | 97009 | 23: 20409761 | 97024 | 23: 20698756 |
| 97025 | 23: 20786968 | 97028 | 23: 20835335 | 97029 | 23: 20835446 | 97045 | 23: 21265221 | 97050 | 23: 21344891 |
| 97056 | 23: 21453135 | 97057 | 23: 21453252 | 97066 | 23: 21870117 | 97085 | 23: 22255326 | 97088 | 23: 22364773 |
| 97113 | 23: 23196063 | 97126 | 23: 23576448 | 97135 | 23: 23755239 | 97136 | 23: 23762207 | 97138 | 23: 23782323 |
| 97148 | 23: 23940335 | 97149 | 23: 23968151 | 97156 | 23: 24092418 | 97164 | 23: 24300414 | 97165 | 23: 24303099 |
| 97167 | 23: 24343369 | 97168 | 23: 24345237 | 97169 | 23: 24352270 | 97173 | 23: 24416257 | 97174 | 23: 24423273 |
| 97179 | 23: 24498401 | 97180 | 23: 24505272 | 97186 | 23: 24683101 | 97187 | 23: 24685752 | 97188 | 23: 24686739 |
| 97189 | 23: 24689461 | 97190 | 23: 24692653 | 97191 | 23: 24699153 | 97192 | 23: 24700095 | 97193 | 23: 24701041 |
| 97194 | 23: 24702797 | 97195 | 23: 24703647 | 97196 | 23: 24704414 | 97197 | 23: 24705785 | 97198 | 23: 24709728 |
| 97200 | 23: 24717517 | 97201 | 23: 24718990 | 97202 | 23: 24723139 | 97203 | 23: 24727842 | 97204 | 23: 24729644 |
| 97205 | 23: 24730615 | 97206 | 23: 24732566 | 97207 | 23: 24733786 | 97208 | 23: 24736986 | 97209 | 23: 24739399 |
| 97210 | 23: 24744157 | 97213 | 23: 24790792 | 97214 | 23: 24794179 | 97216 | 23: 24799157 | 97217 | 23: 24800192 |
| 97218 | 23: 24823698 | 97219 | 23: 24823737 | 97224 | 23: 24880628 | 97226 | 23: 24916092 | 97228 | 23: 24934787 |
| 97229 | 23: 24936283 | 97230 | 23: 24939717 | 97232 | 23: 24975703 | 97240 | 23: 25055772 | 97243 | 23: 25088466 |
| 97244 | 23: 25089919 | 97245 | 23: 25102180 | 97247 | 23: 25129357 | 97248 | 23: 25130486 | 97249 | 23: 25143923 |
| 97250 | 23: 25145004 | 97251 | 23: 25146146 | 97252 | 23: 25156088 | 97253 | 23: 25164433 | 97254 | 23: 25166831 |
| 97257 | 23: 25198505 | 97258 | 23: 25199834 | 97259 | 23: 25200752 | 97260 | 23: 25201563 | 97264 | 23: 25222382 |
| 97265 | 23: 25235072 | 97267 | 23: 25235958 | 97269 | 23: 25250609 | 97271 | 23: 25276948 | 97272 | 23: 25283721 |
| 97273 | 23: 25287389 | 97275 | 23: 25323788 | 97279 | 23: 25354558 | 97280 | 23: 25354656 | 97282 | 23: 25372486 |
| 97286 | 23: 25406424 | 97287 | 23: 25406813 | 97289 | 23: 25433764 | 97292 | 23: 25467868 | 97296 | 23: 25560755 |
| 97298 | 23: 25617278 | 97299 | 23: 25674209 | 97300 | 23: 25680001 | 97301 | 23: 25689132 | 97302 | 23: 25690596 |
| 97303 | 23: 25692139 | 97304 | 23: 25693451 | 97306 | 23: 25715869 | 97308 | 23: 25730240 | 97309 | 23: 25730292 |
| 97310 | 23: 25730339 | 97311 | 23: 25750106 | 97312 | 23: 25755389 | 97313 | 23: 25758772 | 97314 | 23: 25759863 |
| 97315 | 23: 25764182 | 97316 | 23: 25767576 | 97317 | 23: 25780173 | 97318 | 23: 25781822 | 97319 | 23: 25786244 |
| 97320 | 23: 25788400 | 97322 | 23: 25818270 | 97323 | 23: 25819563 | 97324 | 23: 25819612 | 97325 | 23: 25819634 |
| 97327 | 23: 25825333 | 97328 | 23: 25827861 | 97330 | 23: 25834509 | 97331 | 23: 25840572 | 97332 | 23: 25842401 |
| 97335 | 23: 25902950 | 97352 | 23: 26938031 | 97353 | 23: 26943744 | 97354 | 23: 26947613 | 97360 | 23: 27113071 |
| 97361 | 23: 27113077 | 97362 | 23: 27113082 | 97363 | 23: 27113092 | 97364 | 23: 27113093 | 97366 | 23: 27139603 |
| 97367 | 23: 27139619 | 97368 | 23: 27139643 | 97369 | 23: 27141360 | 97370 | 23: 27144341 | 97372 | 23: 27175642 |
| 97373 | 23: 27176260 | 97379 | 23: 27233819 | 97381 | 23: 27306574 | 97382 | 23: 27317678 | 97383 | 23: 27325182 |
| 97384 | 23: 27348931 | 97385 | 23: 27355151 | 97386 | 23: 27356345 | 97387 | 23: 27360333 | 97388 | 23: 27361033 |
| 97389 | 23: 27361247 | 97390 | 23: 27361255 | 97392 | 23: 27385789 | 97393 | 23: 27397427 | 97394 | 23: 27397904 |
| 97395 | 23: 27398878 | 97402 | 23: 27492296 | 97403 | 23: 27493205 | 97404 | 23: 27494111 | 97406 | 23: 27497189 |
| 97407 | 23: 27497222 | 97408 | 23: 27497848 | 97409 | 23: 27497875 | 97410 | 23: 27497880 | 97411 | 23: 27499647 |
| 97412 | 23: 27514878 | 97413 | 23: 27516482 | 97414 | 23: 27522712 | 97415 | 23: 27528967 | 97417 | 23: 27538128 |
| 97419 | 23: 27545924 | 97421 | 23: 27601917 | 97425 | 23: 27635451 | 97426 | 23: 27643343 | 97428 | 23: 27657685 |
| 97431 | 23: 27701786 | 97432 | 23: 27727500 | 97435 | 23: 27774805 | 97436 | 23: 27801348 | 97437 | 23: 27815800 |
| 97438 | 23: 27824468 | 97440 | 23: 27883512 | 97441 | 23: 27910429 | 97442 | 23: 27976925 | 97443 | 23: 27978843 |
| 97445 | 23: 27985061 | 97446 | 23: 27988153 | 97447 | 23: 27988939 | 97449 | 23: 27989644 | 97452 | 23: 28001628 |
| 97453 | 23: 28002243 | 97454 | 23: 28003650 | 97455 | 23: 28006280 | 97456 | 23: 28016941 | 97457 | 23: 28023809 |
| 97458 | 23: 28024215 | 97459 | 23: 28024237 | 97460 | 23: 28029876 | 97461 | 23: 28030196 | 97462 | 23: 28040546 |
| 97463 | 23: 28054764 | 97464 | 23: 28055141 | 97465 | 23: 28055144 | 97466 | 23: 28055147 | 97467 | 23: 28055154 |
| 97468 | 23: 28055203 | 97469 | 23: 28055205 | 97470 | 23: 28056174 | 97471 | 23: 28056245 | 97472 | 23: 28056261 |
| 97473 | 23: 28056279 | 97474 | 23: 28058594 | 97475 | 23: 28084930 | 97476 | 23: 28084959 | 97480 | 23: 28142109 |
| 97481 | 23: 28142880 | 97482 | 23: 28143776 | 97483 | 23: 28147241 | 97485 | 23: 28154077 | 97486 | 23: 28154081 |
| 97487 | 23: 28154567 | 97488 | 23: 28158031 | 97489 | 23: 28162469 | 97490 | 23: 28169587 | 97492 | 23: 28176948 |
| 97493 | 23: 28177862 | 97494 | 23: 28178986 | 97495 | 23: 28180501 | 97496 | 23: 28189617 | 97497 | 23: 28214057 |
| 97499 | 23: 28218286 | 97500 | 23: 28226442 | 97502 | 23: 28247153 | 97504 | 23: 28252270 | 97505 | 23: 28260136 |
| 97506 | 23: 28261564 | 97507 | 23: 28266105 | 97511 | 23: 28289161 | 97512 | 23: 28311837 | 97513 | 23: 28314357 |
| 97514 | 23: 28315369 | 97517 | 23: 28344384 | 97518 | 23: 28345650 | 97520 | 23: 28370592 | 97522 | 23: 28424137 |
| 97523 | 23: 28428619 | 97525 | 23: 28451722 | 97529 | 23: 28527686 | 97530 | 23: 28605105 | 97532 | 23: 28614214 |
| 97533 | 23: 28633036 | 97534 | 23: 28644648 | 97535 | 23: 28667350 | 97536 | 23: 28759020 | 97537 | 23: 28768059 |
| 97538 | 23: 28770375 | 97539 | 23: 28770402 | 97540 | 23: 28770465 | 97541 | 23: 28770499 | 97542 | 23: 28770500 |
| 97543 | 23: 28770527 | 97545 | 23: 28779774 | 97547 | 23: 28784189 | 97549 | 23: 28791635 | 97551 | 23: 28804972 |
| 97552 | 23: 28806779 | 97553 | 23: 28808892 | 97554 | 23: 28811155 | 97555 | 23: 28811333 | 97556 | 23: 28817232 |
| 97557 | 23: 28819161 | 97558 | 23: 28819173 | 97560 | 23: 28820599 | 97561 | 23: 28821000 | 97563 | 23: 28829534 |
| 97564 | 23: 28836068 | 97565 | 23: 28840263 | 97567 | 23: 28855995 | 97568 | 23: 28868586 | 97569 | 23: 28870611 |
| 97570 | 23: 28873879 | 97571 | 23: 28878589 | 97572 | 23: 28881735 | 97574 | 23: 28920628 | 97575 | 23: 28935375 |
| 97576 | 23: 28938250 | 97580 | 23: 28964584 | 97581 | 23: 28967619 | 97582 | 23: 28971358 | 97583 | 23: 28980855 |
| 97584 | 23: 29001677 | 97585 | 23: 29002896 | 97586 | 23: 29003973 | 97587 | 23: 29004861 | 97589 | 23: 29028277 |
| 97591 | 23: 29088900 | 97593 | 23: 29127784 | 97595 | 23: 29155100 | 97598 | 23: 29200199 | 97599 | 23: 29231778 |
| 97604 | 23: 29351343 | 97605 | 23: 29351673 | 97606 | 23: 29387809 | 97608 | 23: 29405131 | 97610 | 23: 29505622 |
| 97618 | 23: 29739156 | 97619 | 23: 29739161 | 97629 | 23: 30015425 | 97633 | 23: 30085624 | 97641 | 23: 30343016 |
| 97643 | 23: 30357104 | 97644 | 23: 30377231 | 97645 | 23: 30377501 | 97646 | 23: 30391263 | 97647 | 23: 30408989 |
| 97648 | 23: 30411874 | 97649 | 23: 30417585 | 97650 | 23: 30422451 | 97651 | 23: 30442710 | 97652 | 23: 30443193 |
| 97653 | 23: 30447941 | 97654 | 23: 30464791 | 97656 | 23: 30478896 | 97660 | 23: 30538186 | 97662 | 23: 30568716 |
| 97669 | 23: 30789137 | 97670 | 23: 30789578 | 97671 | 23: 30789711 | 97685 | 23: 31163020 | 97687 | 23: 31193034 |
| 97688 | 23: 31201200 | 97689 | 23: 31207608 | 97690 | 23: 31237063 | 97691 | 23: 31239018 | 97696 | 23: 31408344 |
| 97697 | 23: 31474420 | 97698 | 23: 31478356 | 97699 | 23: 31478871 | 97706 | 23: 31666638 | 97708 | 23: 31712475 |
| 97709 | 23: 31713030 | 97710 | 23: 31713883 | 97712 | 23: 31795823 | 97716 | 23: 31839586 | 97717 | 23: 31847799 |
| 97718 | 23: 31860518 | 97719 | 23: 31864204 | 97720 | 23: 31883086 | 97721 | 23: 31885522 | 97723 | 23: 31892464 |
| 97724 | 23: 31898107 | 97731 | 23: 32103028 | 97735 | 23: 32155949 | 97748 | 23: 32348823 | 97754 | 23: 32429180 |
| 97755 | 23: 32429299 | 97756 | 23: 32429437 | 97760 | 23: 32498379 | 97763 | 23: 32587067 | 97769 | 23: 32714057 |
| 97776 | 23: 32953339 | 97777 | 23: 32953359 | 97778 | 23: 32954768 | 97779 | 23: 32963026 | 97781 | 23: 32991047 |
| 97782 | 23: 33010094 | 97783 | 23: 33010248 | 97784 | 23: 33017162 | 97793 | 23: 33209372 | 97796 | 23: 33232305 |
| 97809 | 23: 33564304 | 97823 | 23: 33923637 | 97829 | 23: 34147241 | 97832 | 23: 34255293 | 97846 | 23: 34559019 |
| 97847 | 23: 34568421 | 97872 | 23: 35579146 | 97874 | 23: 35643122 | 97882 | 23: 35853906 | 97884 | 23: 35933978 |
| 97897 | 23: 36340613 | 97903 | 23: 36491282 | 97904 | 23: 36522078 | 97911 | 23: 36715268 | 97922 | 23: 37003285 |
| 97923 | 23: 37008791 | 97926 | 23: 37147881 | 97929 | 23: 37226966 | 97931 | 23: 37255744 | 97933 | 23: 37290169 |
| 97934 | 23: 37311602 | 97938 | 23: 37438508 | 97939 | 23: 37509546 | 97948 | 23: 37644241 | 97952 | 23: 37725066 |
| 97954 | 23: 37743052 | 97964 | 23: 38103066 | 97969 | 23: 38331199 | 97970 | 23: 38380507 | 97973 | 23: 38417336 |
| 97995 | 23: 38995562 | 98005 | 23: 39248806 | 98007 | 23: 39300011 | 98009 | 23: 39319903 | 98029 | 23: 39426076 |
| 98034 | 23: 39454222 | 98046 | 23: 39542549 | 98058 | 23: 39582084 | 98065 | 23: 39599289 | 98072 | 23: 39623727 |
| 98074 | 23: 39636483 | 98076 | 23: 39640935 | 98083 | 23: 39789176 | 98085 | 23: 39815156 | 98090 | 23: 39928196 |
| 98093 | 23: 39972622 | 98097 | 23: 40044291 | 98098 | 23: 40064417 | 98106 | 23: 40177352 | 98107 | 23: 40190398 |
| 98109 | 23: 40223350 | 98111 | 23: 40255790 | 98112 | 23: 40270530 | 98116 | 23: 40376606 | 98119 | 23: 40424669 |
| 98121 | 23: 40430850 | 98123 | 23: 40480107 | 98124 | 23: 40500673 | 98125 | 23: 40564407 | 98131 | 23: 40662422 |
| 98133 | 23: 40688993 | 98136 | 23: 40758273 | 98139 | 23: 40791971 | 98156 | 23: 41037376 | 98161 | 23: 41085635 |
| 98163 | 23: 41110764 | 98177 | 23: 41370057 | 98178 | 23: 41371667 | 98179 | 23: 41393226 | 98182 | 23: 41488055 |
| 98198 | 23: 41873620 | 98228 | 23: 42474522 | 98251 | 23: 43050343 | 98257 | 23: 43142746 | 98266 | 23: 43402438 |
| 98282 | 23: 43699449 | 98288 | 23: 43890903 | 98290 | 23: 43922804 | 98325 | 23: 44517863 | 98326 | 23: 44517941 |
| 98332 | 23: 44610396 | 98341 | 23: 44856499 | 98343 | 23: 44879028 | 98344 | 23: 44896794 | 98345 | 23: 44914975 |
| 98346 | 23: 44944349 | 98354 | 23: 45035699 | 98357 | 23: 45057099 | 98358 | 23: 45063024 | 98378 | 23: 45357440 |
| 98380 | 23: 45397415 | 98381 | 23: 45398681 | 98383 | 23: 45430124 | 98384 | 23: 45432927 | 98390 | 23: 45560731 |
| 98404 | 23: 46021030 | 98405 | 23: 46022790 | 98406 | 23: 46022913 | 98407 | 23: 46023921 | 98408 | 23: 46024140 |
| 98419 | 23: 46342707 | 98424 | 23: 46476087 | 98434 | 23: 46728089 | 98439 | 23: 46821712 | 98440 | 23: 46867745 |
| 98445 | 23: 46932972 | 98447 | 23: 46982531 | 98448 | 23: 46982538 | 98449 | 23: 46982544 | 98450 | 23: 46982553 |
| 98451 | 23: 46982569 | 98452 | 23: 46982672 | 98453 | 23: 46982688 | 98469 | 23: 47346112 | 98470 | 23: 47346172 |
| 98472 | 23: 47377820 | 98476 | 23: 47501209 | 98479 | 23: 47512112 | 98480 | 23: 47535388 | 98493 | 23: 47816262 |
| 98494 | 23: 47825640 | 98495 | 23: 47825678 | 98496 | 23: 47826029 | 98497 | 23: 47826560 | 98498 | 23: 47827373 |
| 98500 | 23: 47839983 | 98501 | 23: 47840868 | 98503 | 23: 47845586 | 98504 | 23: 47847036 | 98523 | 23: 48330566 |
| 98529 | 23: 48410101 | 98533 | 23: 48581773 | 98538 | 23: 48634859 | 98545 | 23: 48734960 | 98547 | 23: 48752878 |
| 98555 | 23: 48901109 | 98567 | 23: 49079717 | 98569 | 23: 49107918 | 98574 | 23: 49198514 | 98576 | 23: 49210048 |
| 98584 | 23: 49312924 | 98585 | 23: 49312984 | 98586 | 23: 49312985 | 98587 | 23: 49312986 | 98592 | 23: 49424994 |
| 98593 | 23: 49432035 | 98594 | 23: 49442339 | 98602 | 23: 49590910 | 98622 | 23: 49941067 | 98624 | 23: 49942903 |
| 98625 | 23: 49942904 | 98632 | 23: 50043273 | 98635 | 23: 50078664 | 98636 | 23: 50114133 | 98638 | 23: 50169252 |
| 98642 | 23: 50258011 | 98649 | 23: 50375296 | 98664 | 23: 50638453 | 98671 | 23: 50875493 | 98678 | 23: 50932969 |
| 98680 | 23: 50964263 | 98686 | 23: 51018373 | 98689 | 23: 51079827 | 98691 | 23: 51116781 | 98701 | 23: 51298091 |
| 98711 | 23: 51501609 | 98718 | 23: 51674082 | 98722 | 23: 51744554 | 98728 | 23: 51847760 | 98737 | 23: 52008498 |
| 98741 | 23: 52053452 | 98743 | 23: 52076716 | 98781 | 24: 469885 | 98798 | 24: 880857 | 98799 | 24: 888643 |
| 98802 | 24: 930959 | 98805 | 24: 980184 | 98808 | 24: 1046300 | 98818 | 24: 1170762 | 98819 | 24: 1171661 |
| 98834 | 24: 1570237 | 98851 | 24: 1855510 | 98862 | 24: 2006568 | 98871 | 24: 2265624 | 98872 | 24: 2276656 |
| 98900 | 24: 2945776 | 98902 | 24: 2978064 | 98918 | 24: 3289918 | 98930 | 24: 3538543 | 98933 | 24: 3696514 |
| 98940 | 24: 3908092 | 98942 | 24: 3966055 | 98943 | 24: 3966359 | 98947 | 24: 4007654 | 98948 | 24: 4007678 |
| 98949 | 24: 4012661 | 98950 | 24: 4013403 | 98951 | 24: 4021260 | 98956 | 24: 4164775 | 98957 | 24: 4164916 |
| 98958 | 24: 4164934 | 98959 | 24: 4165918 | 98960 | 24: 4168088 | 98965 | 24: 4188159 | 98970 | 24: 4291963 |
| 98974 | 24: 4399899 | 98980 | 24: 4581542 | 98982 | 24: 4612976 | 98989 | 24: 4963101 | 98995 | 24: 5087606 |
| 99004 | 24: 5305296 | 99005 | 24: 5333407 | 99006 | 24: 5337650 | 99018 | 24: 5642653 | 99026 | 24: 5818978 |
| 99038 | 24: 6264925 | 99076 | 24: 7162546 | 99078 | 24: 7286305 | 99086 | 24: 7467215 | 99111 | 24: 8069004 |
| 99112 | 24: 8108977 | 99113 | 24: 8109036 | 99116 | 24: 8121802 | 99118 | 24: 8175362 | 99119 | 24: 8182146 |
| 99146 | 24: 9005035 | 99151 | 24: 9085950 | 99190 | 24: 9913417 | 99192 | 24: 9977557 | 99276 | 24: 10203460 |
| 99283 | 24: 10336705 | 99286 | 24: 10405660 | 99292 | 24: 10590068 | 99293 | 24: 10590091 | 99311 | 24: 11064065 |
| 99321 | 24: 11243732 | 99322 | 24: 11285722 | 99329 | 24: 11451899 | 99339 | 24: 11738379 | 99359 | 24: 12247620 |
| 99364 | 24: 12466661 | 99365 | 24: 12471212 | 99371 | 24: 12650426 | 99372 | 24: 12652362 | 99373 | 24: 12660326 |
| 99396 | 24: 13228420 | 99405 | 24: 13465765 | 99418 | 24: 13789354 | 99419 | 24: 13790831 | 99420 | 24: 13791730 |
| 99427 | 24: 14119618 | 99459 | 24: 14988931 | 99484 | 24: 15813747 | 99490 | 24: 16186692 | 99501 | 24: 16499172 |
| 99503 | 24: 16506987 | 99509 | 24: 16666243 | 99528 | 24: 17162739 | 99530 | 24: 17208682 | 99543 | 24: 17625014 |
| 99565 | 24: 18353069 | 99574 | 24: 18678355 | 99598 | 24: 19285724 | 99603 | 24: 19437043 | 99607 | 24: 19606961 |
| 99610 | 24: 19713861 | 99615 | 24: 19770957 | 99628 | 24: 20349623 | 99629 | 24: 20358408 | 99632 | 24: 20393549 |
| 99638 | 24: 20499578 | 99639 | 24: 20510429 | 99640 | 24: 20510450 | 99641 | 24: 20510487 | 99642 | 24: 20510488 |
| 99643 | 24: 20510505 | 99644 | 24: 20510579 | 99645 | 24: 20510583 | 99646 | 24: 20510587 | 99647 | 24: 20510597 |
| 99648 | 24: 20510600 | 99649 | 24: 20513392 | 99650 | 24: 20513424 | 99651 | 24: 20513517 | 99652 | 24: 20521127 |
| 99653 | 24: 20532438 | 99654 | 24: 20532441 | 99655 | 24: 20532446 | 99656 | 24: 20532502 | 99657 | 24: 20572686 |
| 99669 | 24: 20847626 | 99671 | 24: 20874971 | 99672 | 24: 20920443 | 99673 | 24: 20920491 | 99674 | 24: 20920727 |
| 99675 | 24: 20922777 | 99676 | 24: 20926914 | 99677 | 24: 20927000 | 99678 | 24: 20927024 | 99679 | 24: 20927059 |
| 99680 | 24: 20929398 | 99681 | 24: 20929449 | 99682 | 24: 20929473 | 99693 | 24: 21272688 | 99695 | 24: 21306399 |
| 99696 | 24: 21375203 | 99697 | 24: 21376904 | 99698 | 24: 21376940 | 99699 | 24: 21376967 | 99700 | 24: 21376972 |
| 99701 | 24: 21378281 | 99702 | 24: 21378321 | 99703 | 24: 21378352 | 99704 | 24: 21378382 | 99705 | 24: 21378383 |
| 99706 | 24: 21378384 | 99708 | 24: 21378440 | 99709 | 24: 21378445 | 99711 | 24: 21395177 | 99712 | 24: 21396591 |
| 99716 | 24: 21432058 | 99717 | 24: 21452284 | 99718 | 24: 21456850 | 99719 | 24: 21471419 | 99723 | 24: 21488223 |
| 99725 | 24: 21504166 | 99729 | 24: 21560026 | 99730 | 24: 21560060 | 99732 | 24: 21564234 | 99733 | 24: 21587057 |
| 99734 | 24: 21590358 | 99735 | 24: 21595564 | 99736 | 24: 21598260 | 99737 | 24: 21605659 | 99738 | 24: 21620362 |
| 99739 | 24: 21667119 | 99740 | 24: 21721258 | 99742 | 24: 21765426 | 99754 | 24: 21940194 | 99756 | 24: 21971534 |
| 99762 | 24: 22139528 | 99769 | 24: 22384770 | 99777 | 24: 22507325 | 99779 | 24: 22552252 | 99786 | 24: 22901733 |
| 99787 | 24: 22901743 | 99788 | 24: 22901792 | 99789 | 24: 22901810 | 99790 | 24: 22901819 | 99795 | 24: 23015622 |
| 99797 | 24: 23048740 | 99798 | 24: 23048839 | 99800 | 24: 23138496 | 99808 | 24: 23304844 | 99809 | 24: 23357754 |
| 99812 | 24: 23476124 | 99840 | 24: 24470786 | 99848 | 24: 24601724 | 99850 | 24: 24651791 | 99856 | 24: 24861946 |
| 99858 | 24: 24942299 | 99859 | 24: 24968711 | 99864 | 24: 25055928 | 99867 | 24: 25166673 | 99870 | 24: 25259209 |
| 99871 | 24: 25291130 | 99875 | 24: 25393251 | 99880 | 24: 25549639 | 99881 | 24: 25550670 | 99882 | 24: 25554087 |
| 99883 | 24: 25555252 | 99884 | 24: 25586246 | 99887 | 24: 25655955 | 99891 | 24: 25728080 | 99892 | 24: 25730700 |
| 99893 | 24: 25732798 | 99895 | 24: 25765374 | 99901 | 24: 25840462 | 99928 | 24: 26414878 | 99929 | 24: 26414942 |
| 99939 | 24: 26752400 | 99960 | 24: 27335211 | 99976 | 24: 27659643 | 99977 | 24: 27671338 | 99978 | 24: 27707920 |
| 99987 | 24: 27976475 | 99992 | 24: 28073538 | 10001 | 24: 28717465 | 10002 | 24: 28990565 | 10002 | 24: 28990603 |
| 10002 | 24: 28990620 | 10003 | 24: 29071264 | 10003 | 24: 29205694 | 10003 | 24: 29294106 | 10004 | 24: 29309403 |
| 10005 | 24: 29593728 | 10005 | 24: 29785127 | 10006 | 24: 30022971 | 10006 | 24: 30028664 | 10006 | 24: 30028736 |
| 10007 | 24: 30132653 | 10008 | 24: 30418113 | 10010 | 24: 30736100 | 10010 | 24: 30756120 | 10010 | 24: 30756121 |
| 10011 | 24: 31302891 | 10011 | 24: 31387621 | 10012 | 24: 31602471 | 10014 | 24: 31910298 | 10015 | 24: 32159647 |
| 10015 | 24: 32159730 | 10016 | 24: 32335210 | 10017 | 24: 32561115 | 10017 | 24: 32732872 | 10017 | 24: 32732892 |
| 10018 | 24: 32732952 | 10018 | 24: 32735243 | 10018 | 24: 32769877 | 10018 | 24: 32810095 | 10018 | 24: 32824750 |
| 10018 | 24: 32826968 | 10019 | 24: 32827828 | 10019 | 24: 32830047 | 10019 | 24: 32830055 | 10019 | 24: 32840723 |
| 10019 | 24: 32857855 | 10019 | 24: 32868393 | 10019 | 24: 32875942 | 10020 | 24: 32894247 | 10020 | 24: 33166170 |
| 10021 | 24: 33227163 | 10021 | 24: 33227191 | 10023 | 24: 33780521 | 10025 | 24: 34326602 | 10025 | 24: 34364918 |
| 10026 | 24: 34614458 | 10026 | 24: 34760712 | 10026 | 24: 34762120 | 10027 | 24: 34813345 | 10028 | 24: 35185894 |
| 10028 | 24: 35185970 | 10029 | 24: 35309422 | 10029 | 24: 35335089 | 10029 | 24: 35472794 | 10032 | 24: 35837548 |
| 10034 | 24: 36589347 | 10034 | 24: 36589477 | 10035 | 24: 36835198 | 10035 | 24: 36988061 | 10036 | 24: 37134995 |
| 10036 | 24: 37239650 | 10036 | 24: 37280274 | 10036 | 24: 37304503 | 10036 | 24: 37306947 | 10037 | 24: 37417740 |
| 10038 | 24: 37592997 | 10038 | 24: 37706524 | 10039 | 24: 37931336 | 10040 | 24: 38475906 | 10040 | 24: 38609603 |
| 10041 | 24: 38809089 | 10042 | 24: 38949417 | 10043 | 24: 39200562 | 10043 | 24: 39308113 | 10043 | 24: 39456209 |
| 10043 | 24: 39464085 | 10044 | 24: 39492963 | 10044 | 24: 39687343 | 10045 | 24: 39707191 | 10045 | 24: 39897863 |
| 10045 | 24: 39902640 | 10046 | 24: 39906046 | 10046 | 24: 39906190 | 10046 | 24: 39913847 | 10047 | 24: 40288339 |
| 10049 | 24: 40342848 | 10049 | 24: 40352389 | 10049 | 24: 40395247 | 10049 | 24: 40408675 | 10050 | 24: 40466974 |
| 10050 | 24: 40545027 | 10050 | 24: 40573682 | 10051 | 24: 40583210 | 10051 | 24: 40583246 | 10051 | 24: 40587358 |
| 10051 | 24: 40746951 | 10052 | 24: 40756834 | 10052 | 24: 40764626 | 10052 | 24: 40781770 | 10052 | 24: 40798538 |
| 10052 | 24: 40803087 | 10053 | 24: 40974776 | 10054 | 24: 41093545 | 10054 | 24: 41180993 | 10054 | 24: 41204807 |
| 10056 | 24: 41805610 | 10057 | 24: 41935257 | 10057 | 24: 41944725 | 10057 | 24: 42049855 | 10058 | 24: 42141779 |
| 10058 | 24: 42231198 | 10058 | 24: 42251623 | 10058 | 24: 42272285 | 10058 | 24: 42276093 | 10059 | 24: 42324465 |
| 10059 | 24: 42659053 | 10060 | 24: 42659123 | 10060 | 24: 42659180 | 10060 | 24: 42659202 | 10060 | 24: 42709614 |
| 10060 | 24: 42721124 | 10060 | 24: 42724162 | 10060 | 24: 42725838 | 10060 | 24: 42726075 | 10060 | 24: 42727665 |
| 10061 | 24: 42768635 | 10061 | 24: 42779119 | 10061 | 24: 42816514 | 10061 | 24: 42881112 | 10063 | 24: 43435997 |
| 10063 | 24: 43500003 | 10064 | 24: 44069431 | 10065 | 24: 44298375 | 10066 | 24: 44375642 | 10066 | 24: 44396418 |
| 10066 | 24: 44471971 | 10067 | 24: 44658910 | 10067 | 24: 44735016 | 10068 | 24: 44775120 | 10068 | 24: 44792435 |
| 10069 | 24: 45080627 | 10070 | 24: 45199003 | 10070 | 24: 45243583 | 10071 | 24: 45357351 | 10071 | 24: 45461858 |
| 10071 | 24: 45489006 | 10071 | 24: 45499596 | 10072 | 24: 45501942 | 10072 | 24: 45537440 | 10072 | 24: 45683209 |
| 10073 | 24: 45738887 | 10073 | 24: 45745474 | 10073 | 24: 45809276 | 10073 | 24: 45824753 | 10074 | 24: 45922636 |
| 10075 | 24: 45923194 | 10075 | 24: 46005539 | 10077 | 24: 46107365 | 10077 | 24: 46128800 | 10077 | 24: 46131144 |
| 10077 | 24: 46200299 | 10078 | 24: 46293419 | 10078 | 24: 46339304 | 10078 | 24: 46339449 | 10078 | 24: 46343493 |
| 10078 | 24: 46349257 | 10079 | 24: 46658398 | 10080 | 24: 46735235 | 10080 | 24: 46748616 | 10080 | 24: 46860941 |
| 10080 | 24: 46863126 | 10080 | 24: 46872067 | 10081 | 24: 46872779 | 10081 | 24: 46872783 | 10081 | 24: 46872835 |
| 10081 | 24: 46874235 | 10082 | 24: 47186952 | 10083 | 24: 47493053 | 10083 | 24: 47504673 | 10083 | 24: 47575681 |
| 10084 | 24: 47792768 | 10084 | 24: 47861526 | 10085 | 24: 47942540 | 10086 | 24: 48132259 | 10086 | 24: 48283076 |
| 10087 | 24: 48398019 | 10087 | 24: 48400794 | 10088 | 24: 48949533 | 10089 | 24: 49153895 | 10089 | 24: 49213874 |
| 10089 | 24: 49220538 | 10090 | 24: 49271697 | 10090 | 24: 49386058 | 10090 | 24: 49461650 | 10091 | 24: 49808792 |
| 10092 | 24: 49949844 | 10092 | 24: 49951357 | 10092 | 24: 49954724 | 10093 | 24: 50278508 | 10093 | 24: 50323489 |
| 10093 | 24: 50330708 | 10094 | 24: 50485176 | 10095 | 24: 50664362 | 10095 | 24: 50886055 | 10096 | 24: 51077466 |
| 10096 | 24: 51079480 | 10098 | 24: 51488836 | 10101 | 24: 51956661 | 10101 | 24: 52119070 | 10102 | 24: 52251430 |
| 10102 | 24: 52257462 | 10102 | 24: 52257497 | 10104 | 24: 52725727 | 10105 | 24: 53039441 | 10105 | 24: 53060437 |
| 10106 | 24: 53471017 | 10107 | 24: 53476039 | 10107 | 24: 53480782 | 10107 | 24: 53485369 | 10107 | 24: 53489339 |
| 10108 | 24: 53695135 | 10109 | 24: 54141567 | 10110 | 24: 54171685 | 10110 | 24: 54227463 | 10111 | 24: 54361200 |
| 10111 | 24: 54366075 | 10112 | 24: 54596604 | 10112 | 24: 54605333 | 10113 | 24: 54678133 | 10113 | 24: 54772296 |
| 10114 | 24: 54837850 | 10114 | 24: 54844868 | 10114 | 24: 54868156 | 10116 | 24: 55295472 | 10116 | 24: 55304369 |
| 10117 | 24: 55700714 | 10118 | 24: 55732464 | 10118 | 24: 55736959 | 10118 | 24: 55739826 | 10118 | 24: 55923426 |
| 10119 | 24: 56024340 | 10119 | 24: 56024373 | 10119 | 24: 56024406 | 10119 | 24: 56098690 | 10119 | 24: 56102284 |
| 10119 | 24: 56102647 | 10120 | 24: 56102653 | 10120 | 24: 56102851 | 10122 | 24: 56688810 | 10122 | 24: 56746296 |
| 10122 | 24: 56746319 | 10123 | 24: 56797648 | 10123 | 24: 56813204 | 10125 | 24: 57240950 | 10125 | 24: 57371376 |
| 10125 | 24: 57399764 | 10126 | 24: 57412925 | 10126 | 24: 57552725 | 10127 | 24: 57558999 | 10127 | 24: 57626126 |
| 10128 | 24: 57747906 | 10128 | 24: 57797863 | 10129 | 24: 58058760 | 10129 | 24: 58124009 | 10131 | 24: 58425089 |
| 10132 | 24: 58468125 | 10132 | 24: 58577110 | 10132 | 24: 58601846 | 10134 | 24: 58910690 | 10135 | 24: 59164671 |
| 10135 | 24: 59196985 | 10136 | 24: 59421526 | 10136 | 24: 59468734 | 10138 | 24: 59829538 | 10142 | 24: 60586093 |
| 10142 | 24: 60623865 | 10142 | 24: 60632706 | 10144 | 24: 60989011 | 10144 | 24: 61029712 | 10144 | 24: 61052179 |
| 10144 | 24: 61119884 | 10145 | 24: 61260912 | 10146 | 24: 61463290 | 10146 | 24: 61466635 | 10146 | 24: 61477326 |
| 10146 | 24: 61528992 | 10147 | 24: 61571637 | 10147 | 24: 61692816 | 10148 | 24: 61746382 | 10150 | 25: 121555 |
| 10150 | 25: 127561 | 10151 | 25: 343439 | 10151 | 25: 373190 | 10152 | 25: 376453 | 10152 | 25: 424297 |
| 10152 | 25: 567175 | 10152 | 25: 572006 | 10152 | 25: 670011 | 10152 | 25: 670169 | 10154 | 25: 950550 |
| 10154 | 25: 1045088 | 10154 | 25: 1087084 | 10154 | 25: 1134920 | 10155 | 25: 1139788 | 10155 | 25: 1177714 |
| 10155 | 25: 1296800 | 10155 | 25: 1324226 | 10156 | 25: 1352107 | 10156 | 25: 1353932 | 10156 | 25: 1354851 |
| 10156 | 25: 1364554 | 10156 | 25: 1364583 | 10156 | 25: 1365638 | 10157 | 25: 1405987 | 10157 | 25: 1476171 |
| 10157 | 25: 1478625 | 10158 | 25: 1649137 | 10158 | 25: 1775261 | 10158 | 25: 1777637 | 10159 | 25: 1777709 |
| 10159 | 25: 1777710 | 10159 | 25: 1777721 | 10159 | 25: 1783080 | 10159 | 25: 1790972 | 10159 | 25: 1791028 |
| 10159 | 25: 1808653 | 10159 | 25: 1818249 | 10160 | 25: 1835147 | 10160 | 25: 1862652 | 10160 | 25: 1883004 |
| 10160 | 25: 1966690 | 10161 | 25: 2003071 | 10162 | 25: 2200434 | 10162 | 25: 2247195 | 10165 | 25: 2376504 |
| 10166 | 25: 2422717 | 10166 | 25: 2477784 | 10166 | 25: 2479395 | 10166 | 25: 2479688 | 10166 | 25: 2479754 |
| 10167 | 25: 2537288 | 10167 | 25: 2538507 | 10167 | 25: 2540535 | 10167 | 25: 2547137 | 10167 | 25: 2548630 |
| 10167 | 25: 2552738 | 10167 | 25: 2553655 | 10167 | 25: 2555768 | 10167 | 25: 2560887 | 10167 | 25: 2561123 |
| 10168 | 25: 2563116 | 10168 | 25: 2564127 | 10168 | 25: 2565592 | 10168 | 25: 2568326 | 10168 | 25: 2573673 |
| 10168 | 25: 2575681 | 10168 | 25: 2578879 | 10168 | 25: 2580437 | 10168 | 25: 2581459 | 10169 | 25: 2582592 |
| 10169 | 25: 2585032 | 10169 | 25: 2586441 | 10170 | 25: 2681110 | 10170 | 25: 2681129 | 10170 | 25: 2687754 |
| 10170 | 25: 2695684 | 10170 | 25: 2695701 | 10170 | 25: 2695795 | 10170 | 25: 2695845 | 10171 | 25: 2695937 |
| 10171 | 25: 2696055 | 10171 | 25: 2696181 | 10171 | 25: 2696410 | 10171 | 25: 2698504 | 10171 | 25: 2699035 |
| 10171 | 25: 2699105 | 10171 | 25: 2739170 | 10171 | 25: 2740282 | 10171 | 25: 2740291 | 10172 | 25: 2743006 |
| 10172 | 25: 2743075 | 10172 | 25: 2743261 | 10172 | 25: 2743406 | 10172 | 25: 2749450 | 10172 | 25: 2762252 |
| 10172 | 25: 2762309 | 10172 | 25: 2775821 | 10173 | 25: 2819065 | 10173 | 25: 2819396 | 10173 | 25: 2819471 |
| 10173 | 25: 2819522 | 10173 | 25: 2819528 | 10173 | 25: 2819546 | 10173 | 25: 2819603 | 10175 | 25: 3210540 |
| 10175 | 25: 3310916 | 10176 | 25: 3363490 | 10176 | 25: 3426286 | 10176 | 25: 3477152 | 10177 | 25: 3524368 |
| 10177 | 25: 3529015 | 10177 | 25: 3534470 | 10177 | 25: 3534510 | 10177 | 25: 3554022 | 10178 | 25: 3581070 |
| 10178 | 25: 3588116 | 10178 | 25: 3595733 | 10178 | 25: 3604536 | 10178 | 25: 3606474 | 10178 | 25: 3625688 |
| 10178 | 25: 3630953 | 10178 | 25: 3640402 | 10178 | 25: 3650316 | 10178 | 25: 3656324 | 10179 | 25: 3701510 |
| 10179 | 25: 3830536 | 10179 | 25: 3851824 | 10180 | 25: 3903874 | 10180 | 25: 4088166 | 10181 | 25: 4278985 |
| 10181 | 25: 4319018 | 10185 | 25: 5024647 | 10186 | 25: 5186113 | 10186 | 25: 5326044 | 10191 | 25: 5967822 |
| 10192 | 25: 6012533 | 10193 | 25: 6175707 | 10193 | 25: 6231384 | 10195 | 25: 6788634 | 10196 | 25: 6838881 |
| 10196 | 25: 6862509 | 10196 | 25: 6862547 | 10198 | 25: 7313080 | 10198 | 25: 7484309 | 10198 | 25: 7517426 |
| 10199 | 25: 7734903 | 10199 | 25: 7840771 | 10199 | 25: 7840811 | 10200 | 25: 7884771 | 10200 | 25: 7905114 |
| 10201 | 25: 7941201 | 10201 | 25: 8064816 | 10202 | 25: 8388133 | 10202 | 25: 8511991 | 10204 | 25: 8788906 |
| 10204 | 25: 8844118 | 10205 | 25: 9197230 | 10206 | 25: 9300591 | 10206 | 25: 9365404 | 10206 | 25: 9378741 |
| 10206 | 25: 9385962 | 10207 | 25: 9502049 | 10207 | 25: 9643363 | 10207 | 25: 9687538 | 10208 | 25: 9776752 |
| 10208 | 25: 9843435 | 10209 | 25: 9993793 | 10209 | 25: 10007062 | 10210 | 25: 10139839 | 10210 | 25: 10198288 |
| 10210 | 25: 10227883 | 10210 | 25: 10272497 | 10211 | 25: 10365727 | 10211 | 25: 10541563 | 10211 | 25: 10541576 |
| 10212 | 25: 10547353 | 10213 | 25: 10832103 | 10213 | 25: 10836311 | 10213 | 25: 10948241 | 10213 | 25: 10951982 |
| 10214 | 25: 11012348 | 10214 | 25: 11110219 | 10214 | 25: 11111271 | 10215 | 25: 11138838 | 10216 | 25: 11274257 |
| 10216 | 25: 11436742 | 10216 | 25: 11437820 | 10217 | 25: 11447709 | 10217 | 25: 11455980 | 10217 | 25: 11549424 |
| 10217 | 25: 11549497 | 10217 | 25: 11570419 | 10220 | 25: 12214562 | 10221 | 25: 12401343 | 10223 | 25: 12943128 |
| 10224 | 25: 13251854 | 10224 | 25: 13339029 | 10224 | 25: 13345472 | 10225 | 25: 13388375 | 10226 | 25: 13601294 |
| 10227 | 25: 14013151 | 10228 | 25: 14073120 | 10228 | 25: 14201547 | 10229 | 25: 14377407 | 10229 | 25: 14409676 |
| 10229 | 25: 14448614 | 10229 | 25: 14511967 | 10230 | 25: 14519922 | 10230 | 25: 14583409 | 10231 | 25: 14703237 |
| 10231 | 25: 14862354 | 10232 | 25: 15092643 | 10233 | 25: 15276545 | 10233 | 25: 15276571 | 10233 | 25: 15370203 |
| 10235 | 25: 15564906 | 10235 | 25: 15633665 | 10235 | 25: 15633709 | 10235 | 25: 15633785 | 10236 | 25: 15912161 |
| 10236 | 25: 15942929 | 10236 | 25: 15943009 | 10237 | 25: 15996288 | 10237 | 25: 16093653 | 10238 | 25: 16219145 |
| 10238 | 25: 16288361 | 10238 | 25: 16288367 | 10239 | 25: 16429229 | 10240 | 25: 16494465 | 10242 | 25: 17127180 |
| 10243 | 25: 17439767 | 10243 | 25: 17446840 | 10244 | 25: 17474953 | 10244 | 25: 17475010 | 10244 | 25: 17483539 |
| 10244 | 25: 17488467 | 10245 | 25: 17838540 | 10246 | 25: 18019077 | 10246 | 25: 18091054 | 10247 | 25: 18283899 |
| 10247 | 25: 18285948 | 10248 | 25: 18730651 | 10248 | 25: 18747611 | 10248 | 25: 18760366 | 10248 | 25: 18798985 |
| 10249 | 25: 18976796 | 10250 | 25: 18988158 | 10253 | 25: 19881913 | 10253 | 25: 19929634 | 10254 | 25: 20012375 |
| 10254 | 25: 20043037 | 10254 | 25: 20085662 | 10255 | 25: 20338297 | 10257 | 25: 21049790 | 10257 | 25: 21139359 |
| 10258 | 25: 21196203 | 10258 | 25: 21260648 | 10258 | 25: 21317697 | 10259 | 25: 21318468 | 10259 | 25: 21327438 |
| 10259 | 25: 21329507 | 10259 | 25: 21405459 | 10260 | 25: 21506832 | 10260 | 25: 21524798 | 10260 | 25: 21532563 |
| 10262 | 25: 22103985 | 10264 | 25: 22376703 | 10264 | 25: 22394285 | 10264 | 25: 22420195 | 10265 | 25: 22670949 |
| 10268 | 25: 23399437 | 10269 | 25: 23750537 | 10271 | 25: 24033949 | 10271 | 25: 24132273 | 10272 | 25: 24242643 |
| 10272 | 25: 24361949 | 10274 | 25: 24619856 | 10274 | 25: 24768804 | 10275 | 25: 24829402 | 10275 | 25: 24857183 |
| 10275 | 25: 24902475 | 10275 | 25: 24915980 | 10275 | 25: 24918063 | 10275 | 25: 24918979 | 10276 | 25: 24918980 |
| 10276 | 25: 24920501 | 10277 | 25: 25159107 | 10277 | 25: 25226497 | 10278 | 25: 25231198 | 10278 | 25: 25252790 |
| 10278 | 25: 25319545 | 10278 | 25: 25319598 | 10278 | 25: 25327412 | 10278 | 25: 25327437 | 10278 | 25: 25355320 |
| 10278 | 25: 25355323 | 10279 | 25: 25370729 | 10280 | 25: 25518754 | 10280 | 25: 25644509 | 10280 | 25: 25729230 |
| 10280 | 25: 25764911 | 10281 | 25: 26048984 | 10283 | 25: 26491613 | 10283 | 25: 26491635 | 10283 | 25: 26491678 |
| 10283 | 25: 26491720 | 10283 | 25: 26491762 | 10284 | 25: 26492004 | 10284 | 25: 26492080 | 10284 | 25: 26492086 |
| 10284 | 25: 26492143 | 10284 | 25: 26493056 | 10284 | 25: 26493215 | 10284 | 25: 26493378 | 10285 | 25: 26873278 |
| 10285 | 25: 26879815 | 10286 | 25: 26957845 | 10286 | 25: 27074581 | 10286 | 25: 27078879 | 10286 | 25: 27079915 |
| 10286 | 25: 27081813 | 10288 | 25: 27471780 | 10288 | 25: 27505633 | 10288 | 25: 27529561 | 10288 | 25: 27546363 |
| 10288 | 25: 27577974 | 10288 | 25: 27610631 | 10289 | 25: 27614146 | 10289 | 25: 27621694 | 10289 | 25: 27623762 |
| 10289 | 25: 27634139 | 10289 | 25: 27647855 | 10289 | 25: 27651029 | 10289 | 25: 27654242 | 10289 | 25: 27671337 |
| 10289 | 25: 27672683 | 10289 | 25: 27677306 | 10290 | 25: 27724958 | 10290 | 25: 27728373 | 10290 | 25: 27731091 |
| 10290 | 25: 27734591 | 10290 | 25: 27737084 | 10290 | 25: 27743420 | 10290 | 25: 27747175 | 10291 | 25: 27747494 |
| 10291 | 25: 27747510 | 10291 | 25: 27748521 | 10291 | 25: 27749865 | 10291 | 25: 27749919 | 10291 | 25: 27749952 |
| 10291 | 25: 27749994 | 10291 | 25: 27750315 | 10291 | 25: 27750769 | 10291 | 25: 27751432 | 10292 | 25: 27790340 |
| 10293 | 25: 28024158 | 10293 | 25: 28057161 | 10293 | 25: 28188815 | 10294 | 25: 28274850 | 10294 | 25: 28376458 |
| 10295 | 25: 28539043 | 10295 | 25: 28760002 | 10297 | 25: 29158123 | 10298 | 25: 29422226 | 10298 | 25: 29507318 |
| 10299 | 25: 29574598 | 10299 | 25: 29653196 | 10300 | 25: 29711516 | 10301 | 25: 29924846 | 10302 | 25: 29960827 |
| 10302 | 25: 29983402 | 10302 | 25: 30108430 | 10303 | 25: 30188316 | 10306 | 25: 31059775 | 10308 | 25: 31397966 |
| 10308 | 25: 31397972 | 10312 | 25: 32101198 | 10312 | 25: 32115103 | 10312 | 25: 32122180 | 10313 | 25: 32225777 |
| 10314 | 25: 32620702 | 10314 | 25: 32636956 | 10315 | 25: 32651385 | 10315 | 25: 32784599 | 10315 | 25: 32785820 |
| 10315 | 25: 32838124 | 10316 | 25: 33064837 | 10316 | 25: 33068854 | 10317 | 25: 33278840 | 10317 | 25: 33325832 |
| 10318 | 25: 33472178 | 10319 | 25: 33614940 | 10319 | 25: 33626966 | 10320 | 25: 33862553 | 10320 | 25: 33918451 |
| 10320 | 25: 33918614 | 10321 | 25: 33933997 | 10321 | 25: 33986857 | 10322 | 25: 34165756 | 10322 | 25: 34324511 |
| 10323 | 25: 34373185 | 10323 | 25: 34380738 | 10323 | 25: 34390163 | 10323 | 25: 34415206 | 10323 | 25: 34416256 |
| 10323 | 25: 34423242 | 10323 | 25: 34430800 | 10324 | 25: 34512804 | 10324 | 25: 34523864 | 10324 | 25: 34541030 |
| 10324 | 25: 34607593 | 10324 | 25: 34616589 | 10325 | 25: 34627570 | 10325 | 25: 34729070 | 10326 | 25: 34979341 |
| 10327 | 25: 34987197 | 10327 | 25: 35035356 | 10327 | 25: 35047480 | 10327 | 25: 35162155 | 10328 | 25: 35220858 |
| 10328 | 25: 35223524 | 10328 | 25: 35224634 | 10329 | 25: 35416991 | 10329 | 25: 35510566 | 10329 | 25: 35538895 |
| 10329 | 25: 35539311 | 10330 | 25: 35574079 | 10330 | 25: 35596429 | 10330 | 25: 35632501 | 10331 | 25: 35695366 |
| 10331 | 25: 35695667 | 10331 | 25: 35695717 | 10331 | 25: 35696225 | 10331 | 25: 35696282 | 10331 | 25: 35697157 |
| 10331 | 25: 35723853 | 10331 | 25: 35726457 | 10331 | 25: 35737565 | 10332 | 25: 35767708 | 10332 | 25: 35774507 |
| 10332 | 25: 35775008 | 10332 | 25: 35784841 | 10332 | 25: 35868596 | 10333 | 25: 35874518 | 10333 | 25: 35930268 |
| 10333 | 25: 35960525 | 10334 | 25: 36192757 | 10334 | 25: 36214845 | 10334 | 25: 36214858 | 10334 | 25: 36215138 |
| 10335 | 25: 36215175 | 10335 | 25: 36215182 | 10335 | 25: 36215303 | 10335 | 25: 36215758 | 10335 | 25: 36215759 |
| 10335 | 25: 36216611 | 10335 | 25: 36216901 | 10335 | 25: 36216911 | 10335 | 25: 36217079 | 10335 | 25: 36217150 |
| 10336 | 25: 36217198 | 10336 | 25: 36217712 | 10336 | 25: 36217751 | 10336 | 25: 36225793 | 10336 | 25: 36225817 |
| 10336 | 25: 36246266 | 10336 | 25: 36249624 | 10337 | 25: 36344903 | 10337 | 25: 36350043 | 10337 | 25: 36360014 |
| 10337 | 25: 36450152 | 10338 | 25: 36450202 | 10338 | 25: 36512847 | 10338 | 25: 36531061 | 10338 | 25: 36535022 |
| 10338 | 25: 36538745 | 10338 | 25: 36584800 | 10339 | 25: 36811728 | 10339 | 25: 36971262 | 10339 | 25: 36978649 |
| 10340 | 25: 36985203 | 10340 | 25: 37031840 | 10340 | 25: 37034930 | 10340 | 25: 37043595 | 10340 | 25: 37046117 |
| 10340 | 25: 37060161 | 10340 | 25: 37061145 | 10341 | 25: 37136607 | 10341 | 25: 37136612 | 10341 | 25: 37138640 |
| 10341 | 25: 37138651 | 10341 | 25: 37138668 | 10341 | 25: 37149974 | 10342 | 25: 37184274 | 10342 | 25: 37205234 |
| 10342 | 25: 37227790 | 10342 | 25: 37241594 | 10342 | 25: 37242352 | 10342 | 25: 37242939 | 10343 | 25: 37242955 |
| 10343 | 25: 37242995 | 10343 | 25: 37243078 | 10343 | 25: 37252529 | 10343 | 25: 37356346 | 10344 | 25: 37389979 |
| 10346 | 25: 37729407 | 10346 | 25: 37784956 | 10347 | 25: 37964578 | 10348 | 25: 38114076 | 10348 | 25: 38114479 |
| 10348 | 25: 38117206 | 10348 | 25: 38118273 | 10348 | 25: 38126701 | 10348 | 25: 38166852 | 10348 | 25: 38190732 |
| 10349 | 25: 38227705 | 10349 | 25: 38233097 | 10349 | 25: 38300677 | 10349 | 25: 38300772 | 10349 | 25: 38300781 |
| 10350 | 25: 38363440 | 10350 | 25: 38394640 | 10350 | 25: 38394741 | 10350 | 25: 38489542 | 10352 | 25: 38856435 |
| 10352 | 25: 38930854 | 10353 | 25: 39028696 | 10353 | 25: 39045580 | 10354 | 25: 39300696 | 10354 | 25: 39317059 |
| 10354 | 25: 39342146 | 10354 | 25: 39345282 | 10354 | 25: 39449897 | 10355 | 25: 39552784 | 10357 | 25: 39918879 |
| 10359 | 25: 40240409 | 10359 | 25: 40240500 | 10360 | 25: 40328748 | 10360 | 25: 40396102 | 10360 | 25: 40553090 |
| 10361 | 25: 40614314 | 10362 | 25: 40720968 | 10362 | 25: 40758343 | 10363 | 25: 40876573 | 10363 | 25: 40901328 |
| 10363 | 25: 40903884 | 10363 | 25: 40948048 | 10364 | 25: 40954468 | 10364 | 25: 40956329 | 10364 | 25: 40960034 |
| 10364 | 25: 40960925 | 10364 | 25: 40979131 | 10364 | 25: 40982964 | 10364 | 25: 40991964 | 10364 | 25: 40998163 |
| 10364 | 25: 41007519 | 10365 | 25: 41009480 | 10365 | 25: 41012986 | 10365 | 25: 41014477 | 10365 | 25: 41015047 |
| 10365 | 25: 41022922 | 10365 | 25: 41028182 | 10365 | 25: 41031444 | 10365 | 25: 41033389 | 10365 | 25: 41034653 |
| 10365 | 25: 41039109 | 10366 | 25: 41046191 | 10366 | 25: 41066639 | 10366 | 25: 41070067 | 10366 | 25: 41074859 |
| 10367 | 25: 41373314 | 10367 | 25: 41416055 | 10367 | 25: 41479611 | 10367 | 25: 41496081 | 10368 | 25: 41719279 |
| 10368 | 25: 41758231 | 10369 | 25: 42027128 | 10369 | 25: 42031008 | 10370 | 25: 42287133 | 10370 | 25: 42290491 |
| 10370 | 25: 42292572 | 10371 | 26: 840412 | 10372 | 26: 1046523 | 10373 | 26: 1202224 | 10374 | 26: 1527005 |
| 10375 | 26: 1774152 | 10375 | 26: 1837613 | 10375 | 26: 1837679 | 10375 | 26: 1837715 | 10378 | 26: 2789785 |
| 10379 | 26: 2795689 | 10379 | 26: 2807861 | 10379 | 26: 2809033 | 10379 | 26: 2810402 | 10379 | 26: 2810457 |
| 10379 | 26: 2810725 | 10379 | 26: 2811451 | 10379 | 26: 2811491 | 10379 | 26: 2811596 | 10379 | 26: 2811603 |
| 10380 | 26: 2811953 | 10380 | 26: 2812213 | 10380 | 26: 2812496 | 10380 | 26: 2834801 | 10380 | 26: 2836372 |
| 10380 | 26: 2838470 | 10380 | 26: 2839812 | 10380 | 26: 2843878 | 10380 | 26: 2847631 | 10380 | 26: 2853005 |
| 10381 | 26: 2869288 | 10381 | 26: 2912970 | 10382 | 26: 3233006 | 10382 | 26: 3276739 | 10382 | 26: 3276794 |
| 10382 | 26: 3347635 | 10382 | 26: 3347724 | 10385 | 26: 3975478 | 10387 | 26: 4592577 | 10389 | 26: 4967710 |
| 10389 | 26: 5039459 | 10390 | 26: 5408963 | 10395 | 26: 6941203 | 10395 | 26: 6944017 | 10396 | 26: 7431785 |
| 10396 | 26: 7456216 | 10396 | 26: 7484390 | 10397 | 26: 7488314 | 10397 | 26: 7495226 | 10397 | 26: 7510638 |
| 10397 | 26: 7591687 | 10397 | 26: 7696472 | 10398 | 26: 7696485 | 10398 | 26: 7727559 | 10399 | 26: 8027593 |
| 10399 | 26: 8048518 | 10400 | 26: 8204339 | 10401 | 26: 8516699 | 10401 | 26: 8538464 | 10402 | 26: 8757164 |
| 10403 | 26: 8943593 | 10403 | 26: 8960699 | 10404 | 26: 9137009 | 10405 | 26: 9666870 | 10406 | 26: 9801561 |
| 10407 | 26: 10180467 | 10407 | 26: 10180516 | 10408 | 26: 10195205 | 10408 | 26: 10195875 | 10408 | 26: 10196016 |
| 10408 | 26: 10197224 | 10408 | 26: 10201006 | 10408 | 26: 10203129 | 10408 | 26: 10208363 | 10408 | 26: 10212949 |
| 10408 | 26: 10214107 | 10408 | 26: 10221720 | 10409 | 26: 10302490 | 10409 | 26: 10305675 | 10410 | 26: 10616738 |
| 10411 | 26: 10731742 | 10412 | 26: 11167367 | 10413 | 26: 11511468 | 10414 | 26: 11702998 | 10414 | 26: 11703000 |
| 10414 | 26: 11703003 | 10414 | 26: 11703009 | 10415 | 26: 11703013 | 10415 | 26: 11703046 | 10415 | 26: 11703059 |
| 10415 | 26: 11703143 | 10417 | 26: 12409311 | 10417 | 26: 12493226 | 10417 | 26: 12493314 | 10417 | 26: 12576243 |
| 10417 | 26: 12663124 | 10418 | 26: 12857727 | 10419 | 26: 12905617 | 10419 | 26: 12936955 | 10419 | 26: 13005236 |
| 10420 | 26: 13185986 | 10420 | 26: 13213326 | 10421 | 26: 13237530 | 10421 | 26: 13242646 | 10421 | 26: 13253767 |
| 10422 | 26: 13348439 | 10422 | 26: 13439428 | 10422 | 26: 13487765 | 10423 | 26: 13487832 | 10423 | 26: 13628978 |
| 10423 | 26: 13632894 | 10423 | 26: 13666033 | 10424 | 26: 13818460 | 10424 | 26: 13935413 | 10425 | 26: 14204588 |
| 10425 | 26: 14204609 | 10427 | 26: 14670368 | 10427 | 26: 14688221 | 10427 | 26: 14729665 | 10427 | 26: 14889689 |
| 10427 | 26: 14890394 | 10428 | 26: 14896553 | 10428 | 26: 15033159 | 10429 | 26: 15470094 | 10431 | 26: 15856645 |
| 10431 | 26: 15856778 | 10431 | 26: 15865811 | 10431 | 26: 15866192 | 10431 | 26: 15867927 | 10431 | 26: 15867943 |
| 10431 | 26: 15876906 | 10434 | 26: 16752906 | 10434 | 26: 16926415 | 10434 | 26: 16926501 | 10435 | 26: 16951340 |
| 10435 | 26: 17033041 | 10435 | 26: 17041251 | 10435 | 26: 17100094 | 10436 | 26: 17127186 | 10436 | 26: 17156417 |
| 10436 | 26: 17197835 | 10436 | 26: 17325459 | 10437 | 26: 17482702 | 10437 | 26: 17539828 | 10438 | 26: 17628730 |
| 10438 | 26: 17628780 | 10438 | 26: 17685991 | 10438 | 26: 17691480 | 10438 | 26: 17691492 | 10439 | 26: 17746468 |
| 10439 | 26: 17781937 | 10439 | 26: 17879952 | 10440 | 26: 17928505 | 10440 | 26: 17965085 | 10441 | 26: 18209480 |
| 10441 | 26: 18211289 | 10441 | 26: 18355673 | 10442 | 26: 18490628 | 10442 | 26: 18494442 | 10442 | 26: 18521176 |
| 10442 | 26: 18528824 | 10443 | 26: 18651237 | 10443 | 26: 18695230 | 10443 | 26: 18702491 | 10443 | 26: 18728512 |
| 10443 | 26: 18740541 | 10444 | 26: 18782784 | 10444 | 26: 18869630 | 10444 | 26: 18885108 | 10444 | 26: 18907964 |
| 10444 | 26: 18929393 | 10445 | 26: 18970293 | 10445 | 26: 19009189 | 10446 | 26: 19238259 | 10447 | 26: 19438464 |
| 10447 | 26: 19452547 | 10447 | 26: 19494159 | 10448 | 26: 19510054 | 10448 | 26: 19547291 | 10448 | 26: 19597530 |
| 10449 | 26: 20104927 | 10449 | 26: 20143627 | 10451 | 26: 20594269 | 10452 | 26: 21094234 | 10453 | 26: 21239671 |
| 10453 | 26: 21254361 | 10456 | 26: 21784164 | 10456 | 26: 21862417 | 10456 | 26: 21918010 | 10456 | 26: 21964449 |
| 10456 | 26: 22002686 | 10457 | 26: 22058857 | 10457 | 26: 22063661 | 10457 | 26: 22251251 | 10458 | 26: 22554517 |
| 10458 | 26: 22581259 | 10458 | 26: 22606617 | 10458 | 26: 22658743 | 10459 | 26: 22659438 | 10461 | 26: 23328557 |
| 10461 | 26: 23398930 | 10461 | 26: 23503177 | 10462 | 26: 23741250 | 10463 | 26: 24028517 | 10463 | 26: 24127485 |
| 10463 | 26: 24135884 | 10464 | 26: 24188588 | 10464 | 26: 24239524 | 10464 | 26: 24286733 | 10464 | 26: 24287516 |
| 10464 | 26: 24310631 | 10465 | 26: 24590237 | 10466 | 26: 24630200 | 10466 | 26: 24764833 | 10466 | 26: 24781597 |
| 10466 | 26: 24818702 | 10467 | 26: 24819776 | 10467 | 26: 24820679 | 10467 | 26: 24823044 | 10467 | 26: 24826317 |
| 10467 | 26: 24829772 | 10467 | 26: 24831082 | 10467 | 26: 24831934 | 10467 | 26: 24850967 | 10468 | 26: 24867806 |
| 10468 | 26: 24877750 | 10468 | 26: 24920605 | 10470 | 26: 25371117 | 10470 | 26: 25377110 | 10471 | 26: 25408794 |
| 10471 | 26: 25438245 | 10472 | 26: 25560669 | 10472 | 26: 25586394 | 10472 | 26: 25601218 | 10472 | 26: 25624158 |
| 10472 | 26: 25628586 | 10472 | 26: 25632049 | 10472 | 26: 25636772 | 10472 | 26: 25637820 | 10472 | 26: 25640830 |
| 10473 | 26: 25643571 | 10473 | 26: 25645847 | 10473 | 26: 25646718 | 10473 | 26: 25649902 | 10473 | 26: 25653351 |
| 10473 | 26: 25664984 | 10473 | 26: 25690676 | 10474 | 26: 25738398 | 10474 | 26: 25884432 | 10474 | 26: 25905471 |
| 10474 | 26: 25923067 | 10474 | 26: 25924666 | 10475 | 26: 26022373 | 10475 | 26: 26108845 | 10476 | 26: 26209053 |
| 10476 | 26: 26211205 | 10477 | 26: 26461336 | 10479 | 26: 26855141 | 10480 | 26: 27069339 | 10482 | 26: 27558387 |
| 10482 | 26: 27572772 | 10483 | 26: 27736462 | 10483 | 26: 27736471 | 10483 | 26: 27736612 | 10485 | 26: 28197657 |
| 10487 | 26: 28950952 | 10487 | 26: 29088929 | 10488 | 26: 29186399 | 10489 | 26: 29513286 | 10491 | 26: 29986030 |
| 10491 | 26: 30197525 | 10491 | 26: 30197564 | 10491 | 26: 30197578 | 10492 | 26: 30197681 | 10493 | 26: 30889276 |
| 10493 | 26: 30892846 | 10494 | 26: 30950051 | 10495 | 26: 31271680 | 10495 | 26: 31311514 | 10495 | 26: 31323087 |
| 10496 | 26: 31350194 | 10496 | 26: 31368850 | 10496 | 26: 31379154 | 10496 | 26: 31411730 | 10496 | 26: 31483017 |
| 10497 | 26: 31801170 | 10499 | 26: 32178829 | 10499 | 26: 32210509 | 10500 | 26: 32484431 | 10500 | 26: 32527484 |
| 10501 | 26: 32691372 | 10501 | 26: 32756815 | 10502 | 26: 32879086 | 10503 | 26: 33122620 | 10504 | 26: 33282896 |
| 10504 | 26: 33328268 | 10504 | 26: 33359761 | 10504 | 26: 33380765 | 10504 | 26: 33400303 | 10505 | 26: 33526762 |
| 10505 | 26: 33530073 | 10505 | 26: 33560198 | 10506 | 26: 33837346 | 10506 | 26: 33901952 | 10507 | 26: 33914479 |
| 10507 | 26: 33922739 | 10508 | 26: 34087101 | 10508 | 26: 34109859 | 10508 | 26: 34125393 | 10508 | 26: 34140428 |
| 10508 | 26: 34145240 | 10508 | 26: 34148000 | 10509 | 26: 34318487 | 10509 | 26: 34354045 | 10510 | 26: 34633936 |
| 10511 | 26: 34718279 | 10511 | 26: 34746698 | 10511 | 26: 34746782 | 10511 | 26: 34746817 | 10511 | 26: 34746843 |
| 10511 | 26: 34746854 | 10511 | 26: 34746898 | 10511 | 26: 34755555 | 10512 | 26: 34829732 | 10512 | 26: 34832045 |
| 10512 | 26: 35009461 | 10513 | 26: 35045257 | 10513 | 26: 35119651 | 10513 | 26: 35152534 | 10514 | 26: 35387482 |
| 10514 | 26: 35446688 | 10515 | 26: 35473524 | 10515 | 26: 35703319 | 10515 | 26: 35730641 | 10515 | 26: 35730653 |
| 10515 | 26: 35730692 | 10515 | 26: 35730695 | 10516 | 26: 35894255 | 10518 | 26: 36442085 | 10518 | 26: 36465890 |
| 10518 | 26: 36466008 | 10519 | 26: 36782486 | 10520 | 26: 36868486 | 10520 | 26: 36875653 | 10520 | 26: 36881092 |
| 10520 | 26: 36895816 | 10520 | 26: 36899560 | 10520 | 26: 36899693 | 10520 | 26: 36955993 | 10520 | 26: 36966986 |
| 10521 | 26: 36968918 | 10521 | 26: 36968980 | 10521 | 26: 36969055 | 10522 | 26: 37095749 | 10523 | 26: 37812473 |
| 10524 | 26: 37879370 | 10524 | 26: 37908929 | 10524 | 26: 38110713 | 10526 | 26: 38674064 | 10527 | 26: 38851055 |
| 10527 | 26: 38854747 | 10527 | 26: 38875810 | 10527 | 26: 38877239 | 10527 | 26: 38881457 | 10528 | 26: 38992651 |
| 10528 | 26: 39006604 | 10529 | 26: 39098131 | 10529 | 26: 39189961 | 10529 | 26: 39196795 | 10529 | 26: 39202461 |
| 10530 | 26: 39305962 | 10530 | 26: 39426186 | 10530 | 26: 39426206 | 10530 | 26: 39426264 | 10530 | 26: 39485796 |
| 10531 | 26: 39494577 | 10531 | 26: 39507180 | 10531 | 26: 39538069 | 10531 | 26: 39573736 | 10532 | 26: 39783330 |
| 10532 | 26: 40032776 | 10533 | 26: 40040946 | 10533 | 26: 40070046 | 10534 | 26: 40441942 | 10535 | 26: 40786447 |
| 10536 | 26: 41019385 | 10536 | 26: 41076854 | 10536 | 26: 41096640 | 10537 | 26: 41390989 | 10539 | 26: 41918819 |
| 10539 | 26: 41926321 | 10540 | 26: 42008210 | 10540 | 26: 42024771 | 10540 | 26: 42049951 | 10540 | 26: 42090283 |
| 10540 | 26: 42092905 | 10541 | 26: 42109007 | 10541 | 26: 42309356 | 10541 | 26: 42330778 | 10542 | 26: 42350916 |
| 10543 | 26: 42642591 | 10543 | 26: 42660447 | 10543 | 26: 42757714 | 10544 | 26: 43041112 | 10545 | 26: 43062676 |
| 10546 | 26: 43622892 | 10547 | 26: 43740696 | 10547 | 26: 43816442 | 10548 | 26: 44064506 | 10548 | 26: 44083273 |
| 10548 | 26: 44083285 | 10549 | 26: 44083315 | 10549 | 26: 44083393 | 10549 | 26: 44083680 | 10550 | 26: 44255933 |
| 10550 | 26: 44404294 | 10550 | 26: 44481659 | 10551 | 26: 44674003 | 10551 | 26: 44723893 | 10552 | 26: 45008042 |
| 10553 | 26: 45209100 | 10553 | 26: 45264287 | 10554 | 26: 45324531 | 10555 | 26: 45412608 | 10556 | 26: 45540372 |
| 10556 | 26: 45540404 | 10556 | 26: 45540418 | 10556 | 26: 45540427 | 10556 | 26: 45542714 | 10556 | 26: 45565711 |
| 10557 | 26: 45729275 | 10558 | 26: 46112041 | 10559 | 26: 46315775 | 10559 | 26: 46317103 | 10559 | 26: 46317812 |
| 10559 | 26: 46318923 | 10560 | 26: 46353080 | 10560 | 26: 46421801 | 10560 | 26: 46452461 | 10560 | 26: 46466580 |
| 10560 | 26: 46471616 | 10560 | 26: 46479016 | 10561 | 26: 46559579 | 10563 | 26: 47028707 | 10564 | 26: 47333154 |
| 10564 | 26: 47361635 | 10564 | 26: 47427206 | 10565 | 26: 47512133 | 10565 | 26: 47512681 | 10565 | 26: 47615883 |
| 10566 | 26: 47650142 | 10566 | 26: 47772970 | 10569 | 26: 48408440 | 10569 | 26: 48479152 | 10571 | 26: 48865592 |
| 10571 | 26: 48866736 | 10571 | 26: 48968839 | 10571 | 26: 49004191 | 10574 | 26: 49515195 | 10574 | 26: 49563646 |
| 10575 | 26: 49563747 | 10579 | 26: 50590249 | 10579 | 26: 50709285 | 10579 | 26: 50733543 | 10580 | 26: 50733621 |
| 10580 | 26: 50823585 | 10580 | 26: 50825362 | 10580 | 26: 50830075 | 10581 | 26: 51009547 | 10581 | 26: 51009628 |
| 10581 | 26: 51127593 | 10582 | 26: 51175800 | 10583 | 26: 51422136 | 10584 | 26: 51748308 | 10584 | 26: 51784240 |
| 10584 | 26: 51824417 | 10584 | 26: 51837838 | 10584 | 26: 51856701 | 10585 | 26: 51990316 | 10586 | 27: 504917 |
| 10586 | 27: 504918 | 10586 | 27: 504943 | 10586 | 27: 622508 | 10587 | 27: 1220407 | 10588 | 27: 1285546 |
| 10588 | 27: 1330181 | 10588 | 27: 1355435 | 10589 | 27: 1355462 | 10589 | 27: 1362304 | 10589 | 27: 1362959 |
| 10589 | 27: 1375496 | 10589 | 27: 1376218 | 10589 | 27: 1382415 | 10589 | 27: 1384981 | 10589 | 27: 1385318 |
| 10589 | 27: 1385323 | 10589 | 27: 1385392 | 10590 | 27: 1385425 | 10590 | 27: 1385455 | 10590 | 27: 1395995 |
| 10590 | 27: 1401856 | 10590 | 27: 1401865 | 10590 | 27: 1405119 | 10590 | 27: 1407822 | 10590 | 27: 1453147 |
| 10591 | 27: 1475170 | 10593 | 27: 2200746 | 10593 | 27: 2232971 | 10594 | 27: 2375493 | 10595 | 27: 2508576 |
| 10597 | 27: 2988336 | 10600 | 27: 3776760 | 10600 | 27: 3787074 | 10600 | 27: 3788517 | 10600 | 27: 3790934 |
| 10600 | 27: 3805789 | 10602 | 27: 4234197 | 10604 | 27: 4820720 | 10604 | 27: 4843559 | 10605 | 27: 4942987 |
| 10605 | 27: 4946073 | 10605 | 27: 4962502 | 10605 | 27: 4964043 | 10605 | 27: 5085626 | 10606 | 27: 5098545 |
| 10606 | 27: 5158253 | 10606 | 27: 5391087 | 10607 | 27: 5392011 | 10607 | 27: 5395069 | 10607 | 27: 5396567 |
| 10607 | 27: 5399954 | 10607 | 27: 5401556 | 10607 | 27: 5402389 | 10607 | 27: 5403342 | 10607 | 27: 5404401 |
| 10607 | 27: 5418944 | 10608 | 27: 5421637 | 10608 | 27: 5428881 | 10608 | 27: 5430538 | 10608 | 27: 5470087 |
| 10608 | 27: 5478901 | 10608 | 27: 5582204 | 10609 | 27: 5587883 | 10609 | 27: 5611632 | 10609 | 27: 5792112 |
| 10610 | 27: 5827416 | 10610 | 27: 5974973 | 10610 | 27: 5988748 | 10610 | 27: 6025859 | 10610 | 27: 6032871 |
| 10611 | 27: 6060484 | 10611 | 27: 6087111 | 10614 | 27: 7631148 | 10614 | 27: 7653356 | 10614 | 27: 7733608 |
| 10615 | 27: 7750098 | 10615 | 27: 7770345 | 10615 | 27: 7791683 | 10615 | 27: 7807058 | 10615 | 27: 7813201 |
| 10616 | 27: 7920370 | 10616 | 27: 7920432 | 10616 | 27: 7920451 | 10616 | 27: 7958336 | 10617 | 27: 7975782 |
| 10617 | 27: 7980889 | 10617 | 27: 8132488 | 10617 | 27: 8191553 | 10618 | 27: 8289670 | 10618 | 27: 8359908 |
| 10618 | 27: 8470036 | 10618 | 27: 8476834 | 10619 | 27: 8575128 | 10619 | 27: 8612967 | 10623 | 27: 9145723 |
| 10624 | 27: 9202230 | 10624 | 27: 9202257 | 10624 | 27: 9202263 | 10624 | 27: 9202295 | 10625 | 27: 9488574 |
| 10630 | 27: 11255396 | 10630 | 27: 11262348 | 10632 | 27: 11669357 | 10632 | 27: 11736207 | 10634 | 27: 12235101 |
| 10634 | 27: 12270834 | 10637 | 27: 12976567 | 10637 | 27: 13162684 | 10638 | 27: 13319931 | 10639 | 27: 13521583 |
| 10640 | 27: 13703268 | 10640 | 27: 13763652 | 10641 | 27: 13769140 | 10641 | 27: 13864569 | 10645 | 27: 14058907 |
| 10645 | 27: 14089539 | 10646 | 27: 14337753 | 10647 | 27: 14505664 | 10647 | 27: 14619598 | 10650 | 27: 15114953 |
| 10650 | 27: 15209444 | 10651 | 27: 15342336 | 10651 | 27: 15374174 | 10652 | 27: 15616842 | 10653 | 27: 15679545 |
| 10653 | 27: 15679907 | 10653 | 27: 15679986 | 10653 | 27: 15679987 | 10653 | 27: 15724630 | 10655 | 27: 16091144 |
| 10656 | 27: 16231107 | 10656 | 27: 16231139 | 10656 | 27: 16250819 | 10656 | 27: 16262844 | 10657 | 27: 16289197 |
| 10657 | 27: 16289212 | 10657 | 27: 16310423 | 10657 | 27: 16311908 | 10657 | 27: 16311992 | 10658 | 27: 16448657 |
| 10658 | 27: 16475268 | 10658 | 27: 16527145 | 10658 | 27: 16527632 | 10659 | 27: 16545436 | 10659 | 27: 16626449 |
| 10660 | 27: 16876243 | 10661 | 27: 17114420 | 10663 | 27: 17756331 | 10663 | 27: 17825429 | 10664 | 27: 17900781 |
| 10668 | 27: 18743674 | 10670 | 27: 19184326 | 10670 | 27: 19226515 | 10671 | 27: 19413103 | 10672 | 27: 19615386 |
| 10672 | 27: 19628648 | 10672 | 27: 19638432 | 10672 | 27: 19643687 | 10672 | 27: 19643703 | 10672 | 27: 19656115 |
| 10673 | 27: 19709555 | 10673 | 27: 19715294 | 10673 | 27: 19715322 | 10673 | 27: 19743997 | 10673 | 27: 19759721 |
| 10674 | 27: 19916100 | 10675 | 27: 20275887 | 10676 | 27: 20403248 | 10677 | 27: 21028770 | 10679 | 27: 21603965 |
| 10680 | 27: 21736051 | 10681 | 27: 21968621 | 10682 | 27: 21988370 | 10682 | 27: 21988471 | 10682 | 27: 22006706 |
| 10683 | 27: 22555224 | 10686 | 27: 23217717 | 10686 | 27: 23219768 | 10686 | 27: 23235008 | 10686 | 27: 23291834 |
| 10688 | 27: 23591760 | 10688 | 27: 23763903 | 10689 | 27: 23820540 | 10689 | 27: 23900777 | 10690 | 27: 23960140 |
| 10690 | 27: 23969325 | 10690 | 27: 23997295 | 10690 | 27: 24130313 | 10691 | 27: 24151993 | 10691 | 27: 24152004 |
| 10691 | 27: 24203304 | 10691 | 27: 24203421 | 10691 | 27: 24222816 | 10692 | 27: 24521008 | 10693 | 27: 24725230 |
| 10694 | 27: 24839161 | 10694 | 27: 24839261 | 10696 | 27: 25252926 | 10696 | 27: 25328242 | 10697 | 27: 25416389 |
| 10698 | 27: 25562534 | 10698 | 27: 25566427 | 10698 | 27: 25728403 | 10699 | 27: 26089588 | 10700 | 27: 26265901 |
| 10700 | 27: 26306516 | 10700 | 27: 26385003 | 10701 | 27: 26490500 | 10702 | 27: 26581172 | 10702 | 27: 26586255 |
| 10702 | 27: 26593729 | 10703 | 27: 26832000 | 10704 | 27: 26846204 | 10704 | 27: 26878354 | 10704 | 27: 26886076 |
| 10704 | 27: 26894469 | 10704 | 27: 26904559 | 10704 | 27: 26906127 | 10704 | 27: 26924212 | 10706 | 27: 27222640 |
| 10706 | 27: 27223927 | 10706 | 27: 27223929 | 10706 | 27: 27224038 | 10706 | 27: 27224048 | 10706 | 27: 27224849 |
| 10706 | 27: 27224850 | 10708 | 27: 27637602 | 10709 | 27: 27970577 | 10710 | 27: 28171888 | 10713 | 27: 28946541 |
| 10714 | 27: 29252315 | 10715 | 27: 29278521 | 10715 | 27: 29296483 | 10715 | 27: 29312772 | 10715 | 27: 29327883 |
| 10715 | 27: 29327914 | 10716 | 27: 29327998 | 10718 | 27: 30202829 | 10719 | 27: 30202912 | 10719 | 27: 30226393 |
| 10719 | 27: 30244634 | 10722 | 27: 31002073 | 10722 | 27: 31002095 | 10722 | 27: 31002129 | 10723 | 27: 31078802 |
| 10724 | 27: 31274065 | 10726 | 27: 31729965 | 10726 | 27: 31841660 | 10726 | 27: 31841704 | 10727 | 27: 31897740 |
| 10729 | 27: 32226765 | 10729 | 27: 32254312 | 10729 | 27: 32265770 | 10730 | 27: 32268130 | 10730 | 27: 32276493 |
| 10731 | 27: 32518470 | 10731 | 27: 32518472 | 10731 | 27: 32518552 | 10731 | 27: 32583194 | 10732 | 27: 32727516 |
| 10732 | 27: 32727582 | 10732 | 27: 32727603 | 10732 | 27: 32727620 | 10732 | 27: 32778474 | 10732 | 27: 32793041 |
| 10733 | 27: 32802126 | 10734 | 27: 33001000 | 10734 | 27: 33009928 | 10734 | 27: 33086268 | 10735 | 27: 33233127 |
| 10735 | 27: 33265366 | 10735 | 27: 33272590 | 10736 | 27: 33421612 | 10736 | 27: 33629831 | 10736 | 27: 33633260 |
| 10737 | 27: 33872186 | 10737 | 27: 33879901 | 10737 | 27: 33913097 | 10738 | 27: 34127235 | 10739 | 27: 34635911 |
| 10740 | 27: 34637563 | 10740 | 27: 34752720 | 10741 | 27: 34981815 | 10741 | 27: 35014810 | 10741 | 27: 35015975 |
| 10741 | 27: 35016903 | 10741 | 27: 35018328 | 10741 | 27: 35019984 | 10741 | 27: 35021367 | 10742 | 27: 35026180 |
| 10742 | 27: 35032183 | 10742 | 27: 35042729 | 10742 | 27: 35045715 | 10742 | 27: 35047820 | 10742 | 27: 35052712 |
| 10742 | 27: 35053785 | 10742 | 27: 35054739 | 10742 | 27: 35058765 | 10743 | 27: 35060134 | 10743 | 27: 35064362 |
| 10743 | 27: 35065072 | 10743 | 27: 35072059 | 10743 | 27: 35075103 | 10743 | 27: 35079318 | 10743 | 27: 35080042 |
| 10743 | 27: 35098005 | 10744 | 27: 35103784 | 10744 | 27: 35121417 | 10744 | 27: 35122313 | 10744 | 27: 35128376 |
| 10744 | 27: 35138175 | 10744 | 27: 35184790 | 10747 | 27: 35555630 | 10747 | 27: 35599360 | 10748 | 27: 35782254 |
| 10748 | 27: 35824524 | 10748 | 27: 35824606 | 10749 | 27: 35975385 | 10749 | 27: 35978336 | 10750 | 27: 36138112 |
| 10750 | 27: 36216370 | 10752 | 27: 36576641 | 10753 | 27: 36751396 | 10753 | 27: 36906500 | 10753 | 27: 37106071 |
| 10754 | 27: 37127877 | 10754 | 27: 37172084 | 10754 | 27: 37192870 | 10754 | 27: 37195004 | 10754 | 27: 37274132 |
| 10755 | 27: 37275861 | 10755 | 27: 37293040 | 10755 | 27: 37297500 | 10756 | 27: 37374389 | 10756 | 27: 37486971 |
| 10756 | 27: 37493520 | 10756 | 27: 37521766 | 10757 | 27: 37573787 | 10757 | 27: 37620840 | 10757 | 27: 37630299 |
| 10757 | 27: 37639061 | 10758 | 27: 37746356 | 10759 | 27: 37827637 | 10759 | 27: 37864645 | 10760 | 27: 38151557 |
| 10760 | 27: 38168061 | 10761 | 27: 38327204 | 10761 | 27: 38432520 | 10762 | 27: 38491615 | 10763 | 27: 38861489 |
| 10764 | 27: 39015604 | 10764 | 27: 39015763 | 10764 | 27: 39015976 | 10766 | 27: 39667527 | 10766 | 27: 39668431 |
| 10767 | 27: 39780803 | 10768 | 27: 40004933 | 10769 | 27: 40356156 | 10769 | 27: 40357028 | 10769 | 27: 40414746 |
| 10770 | 27: 40674026 | 10770 | 27: 40868181 | 10771 | 27: 41044067 | 10772 | 27: 41178088 | 10773 | 27: 41583577 |
| 10774 | 27: 41702146 | 10775 | 27: 41886684 | 10776 | 27: 42166403 | 10776 | 27: 42228982 | 10776 | 27: 42242635 |
| 10777 | 27: 42427599 | 10777 | 27: 42462522 | 10777 | 27: 42487443 | 10778 | 27: 42794556 | 10781 | 27: 43428164 |
| 10783 | 27: 43823427 | 10783 | 27: 43863511 | 10783 | 27: 43908553 | 10784 | 27: 43927325 | 10784 | 27: 43987089 |
| 10785 | 27: 43995458 | 10785 | 27: 44200677 | 10787 | 27: 44375041 | 10787 | 27: 44412495 | 10788 | 27: 44543531 |
| 10788 | 27: 44629778 | 10792 | 28: 479125 | 10792 | 28: 504407 | 10793 | 28: 692680 | 10793 | 28: 704706 |
| 10793 | 28: 704958 | 10794 | 28: 1196781 | 10795 | 28: 1230989 | 10795 | 28: 1234367 | 10795 | 28: 1243626 |
| 10795 | 28: 1243641 | 10795 | 28: 1243692 | 10795 | 28: 1320241 | 10796 | 28: 1399889 | 10796 | 28: 1487331 |
| 10796 | 28: 1489641 | 10798 | 28: 1783556 | 10798 | 28: 1818831 | 10798 | 28: 1840156 | 10799 | 28: 1924353 |
| 10799 | 28: 1964866 | 10799 | 28: 2032083 | 10800 | 28: 2144906 | 10800 | 28: 2162175 | 10801 | 28: 2343692 |
| 10802 | 28: 2709799 | 10802 | 28: 2730701 | 10803 | 28: 3272910 | 10803 | 28: 3476063 | 10803 | 28: 3548826 |
| 10803 | 28: 3590983 | 10804 | 28: 3973141 | 10804 | 28: 3973211 | 10804 | 28: 3984834 | 10805 | 28: 4431180 |
| 10805 | 28: 4631548 | 10806 | 28: 4701678 | 10806 | 28: 4907755 | 10807 | 28: 5014037 | 10808 | 28: 5220057 |
| 10808 | 28: 5220146 | 10808 | 28: 5220186 | 10808 | 28: 5245742 | 10808 | 28: 5247877 | 10808 | 28: 5253360 |
| 1081C | 28: 5630191 | 10810 | 28: 5815635 | 10810 | 28: 5833150 | 10811 | 28: 5839078 | 10811 | 28: 5870781 |
| 10811 | 28: 5870881 | 10811 | 28: 5873548 | 10811 | 28: 5873612 | 10811 | 28: 5873655 | 10811 | 28: 5874287 |
| 10811 | 28: 5903348 | 10814 | 28: 6691792 | 10815 | 28: 6866814 | 10815 | 28: 6866847 | 10815 | 28: 6866914 |
| 10816 | 28: 7257360 | 10817 | 28: 7304728 | 10817 | 28: 7321818 | 10817 | 28: 7339237 | 10817 | 28: 7378556 |
| 10817 | 28: 7415736 | 10818 | 28: 7555026 | 10818 | 28: 7760692 | 10819 | 28: 7786515 | 10819 | 28: 7813529 |
| 10820 | 28: 8105292 | 10820 | 28: 8125888 | 10821 | 28: 8201519 | 10821 | 28: 8331969 | 10822 | 28: 8547431 |
| 10823 | 28: 8650652 | 10823 | 28: 8698842 | 10823 | 28: 8707863 | 10824 | 28: 8843955 | 10824 | 28: 9025349 |
| 10825 | 28: 9100351 | 10825 | 28: 9269631 | 10826 | 28: 9336102 | 10826 | 28: 9386021 | 10826 | 28: 9586664 |
| 10828 | 28: 9807554 | 10828 | 28: 9913957 | 10828 | 28: 9914017 | 10828 | 28: 9915219 | 10828 | 28: 9915233 |
| 10830 | 28: 10277520 | 10835 | 28: 12225835 | 10837 | 28: 12371401 | 10838 | 28: 12623784 | 10838 | 28: 12623831 |
| 10838 | 28: 12623855 | 10840 | 28: 13022825 | 10841 | 28: 13252341 | 10843 | 28: 13950890 | 10844 | 28: 14104824 |
| 10844 | 28: 14144978 | 10844 | 28: 14157633 | 10844 | 28: 14181805 | 10844 | 28: 14204540 | 10845 | 28: 14219987 |
| 10845 | 28: 14342309 | 10845 | 28: 14415424 | 10845 | 28: 14415472 | 10846 | 28: 14547089 | 10846 | 28: 14552705 |
| 10847 | 28: 14925580 | 10848 | 28: 14960497 | 10848 | 28: 15003137 | 10849 | 28: 15386201 | 10849 | 28: 15392967 |
| 10850 | 28: 15541195 | 10850 | 28: 15543708 | 10850 | 28: 15562551 | 10851 | 28: 15866947 | 10851 | 28: 15875883 |
| 10851 | 28: 15913989 | 10851 | 28: 15913997 | 10852 | 28: 15914061 | 10852 | 28: 15914091 | 10852 | 28: 15942493 |
| 10852 | 28: 15972055 | 10852 | 28: 16010478 | 10853 | 28: 16039905 | 10853 | 28: 16124447 | 10853 | 28: 16124496 |
| 10854 | 28: 16335973 | 10854 | 28: 16387453 | 10854 | 28: 16407912 | 10855 | 28: 16520465 | 10855 | 28: 16588164 |
| 10855 | 28: 16644285 | 10855 | 28: 16644893 | 10855 | 28: 16648829 | 10856 | 28: 16661175 | 10856 | 28: 16661840 |
| 10856 | 28: 16692080 | 10856 | 28: 16767636 | 10856 | 28: 16773322 | 10856 | 28: 16818843 | 10857 | 28: 16930270 |
| 10858 | 28: 17215906 | 10858 | 28: 17282866 | 10859 | 28: 17285666 | 10859 | 28: 17285711 | 10860 | 28: 17588476 |
| 10861 | 28: 17835910 | 10862 | 28: 18019905 | 10862 | 28: 18019907 | 10862 | 28: 18019913 | 10862 | 28: 18019925 |
| 10863 | 28: 18086730 | 10863 | 28: 18107994 | 10864 | 28: 18485866 | 10866 | 28: 18937069 | 10866 | 28: 19041963 |
| 10866 | 28: 19127485 | 10867 | 28: 19229652 | 10868 | 28: 19534804 | 10868 | 28: 19583995 | 10868 | 28: 19673238 |
| 10869 | 28: 19774362 | 10871 | 28: 20415689 | 10875 | 28: 21574143 | 10878 | 28: 22399855 | 10878 | 28: 22479622 |
| 10879 | 28: 22620494 | 10879 | 28: 22620545 | 10879 | 28: 22620582 | 10880 | 28: 22847773 | 10880 | 28: 23007374 |
| 10880 | 28: 23007384 | 10880 | 28: 23049818 | 10881 | 28: 23173525 | 10881 | 28: 23177219 | 10882 | 28: 23292046 |
| 10882 | 28: 23296757 | 10882 | 28: 23385467 | 10883 | 28: 23385512 | 10885 | 28: 24001450 | 10886 | 28: 24160079 |
| 10886 | 28: 24160811 | 10886 | 28: 24242145 | 10886 | 28: 24253020 | 10888 | 28: 24623959 | 10889 | 28: 24802411 |
| 10889 | 28: 24802550 | 10889 | 28: 24802551 | 10889 | 28: 24885896 | 10890 | 28: 24934653 | 10890 | 28: 25064734 |
| 10890 | 28: 25090839 | 10890 | 28: 25139537 | 10891 | 28: 25385010 | 10891 | 28: 25408853 | 10892 | 28: 25415208 |
| 10892 | 28: 25614547 | 10892 | 28: 25625624 | 10893 | 28: 25625649 | 10893 | 28: 25625667 | 10893 | 28: 25626954 |
| 10893 | 28: 25731593 | 10893 | 28: 25808647 | 10894 | 28: 25947218 | 10894 | 28: 26030383 | 10894 | 28: 26036200 |
| 10895 | 28: 26155221 | 10895 | 28: 26180213 | 10896 | 28: 26181587 | 10896 | 28: 26181622 | 10896 | 28: 26181655 |
| 10896 | 28: 26185280 | 10896 | 28: 26185344 | 10896 | 28: 26185376 | 10896 | 28: 26186082 | 10896 | 28: 26186104 |
| 10896 | 28: 26186116 | 10896 | 28: 26186150 | 10898 | 28: 26504153 | 10898 | 28: 26606263 | 10899 | 28: 26681661 |
| 10899 | 28: 26740880 | 10899 | 28: 26752280 | 10899 | 28: 26814555 | 10900 | 28: 27066599 | 10900 | 28: 27068380 |
| 10901 | 28: 27070779 | 10901 | 28: 27146373 | 10902 | 28: 27317961 | 10903 | 28: 27518059 | 10903 | 28: 27566739 |
| 10904 | 28: 27704605 | 10904 | 28: 27851635 | 10905 | 28: 27937188 | 10905 | 28: 27937338 | 10905 | 28: 27937971 |
| 10906 | 28: 27941915 | 10906 | 28: 27941916 | 10906 | 28: 27941932 | 10906 | 28: 27943776 | 10906 | 28: 27943868 |
| 10906 | 28: 27943901 | 10906 | 28: 27943916 | 10906 | 28: 27948642 | 10906 | 28: 27948643 | 10907 | 28: 27954047 |
| 10907 | 28: 28109673 | 10908 | 28: 28192038 | 10908 | 28: 28290243 | 10909 | 28: 28290255 | 10909 | 28: 28290934 |
| 10909 | 28: 28290955 | 10909 | 28: 28290999 | 10909 | 28: 28313393 | 10910 | 28: 28407860 | 10910 | 28: 28646964 |
| 10910 | 28: 28722550 | 10911 | 28: 28920066 | 10911 | 28: 28920811 | 10911 | 28: 28920813 | 10913 | 28: 29172597 |
| 10913 | 28: 29210804 | 10913 | 28: 29213687 | 10913 | 28: 29236992 | 10913 | 28: 29298781 | 10915 | 28: 29704456 |
| 10915 | 28: 29735862 | 10915 | 28: 29737399 | 10915 | 28: 29776190 | 10916 | 28: 29777653 | 10916 | 28: 29841383 |
| 10916 | 28: 29914673 | 10917 | 28: 30046702 | 10917 | 28: 30226392 | 10918 | 28: 30648480 | 10919 | 28: 30730091 |
| 10919 | 28: 30770433 | 10919 | 28: 30824174 | 10920 | 28: 30869446 | 10920 | 28: 30999414 | 10920 | 28: 31017300 |
| 10920 | 28: 31042068 | 10921 | 28: 31093235 | 10921 | 28: 31386148 | 10922 | 28: 31501902 | 10922 | 28: 31528266 |
| 10922 | 28: 31531949 | 10923 | 28: 31783641 | 10925 | 28: 32102216 | 10925 | 28: 32102950 | 10925 | 28: 32104334 |
| 10925 | 28: 32119157 | 10925 | 28: 32119371 | 10925 | 28: 32122100 | 10925 | 28: 32141403 | 10926 | 28: 32322725 |
| 10927 | 28: 32385967 | 10927 | 28: 32392342 | 10930 | 28: 32904173 | 10930 | 28: 32960277 | 10931 | 28: 32997331 |
| 10931 | 28: 33041333 | 10932 | 28: 33158395 | 10932 | 28: 33186798 | 10932 | 28: 33225867 | 10932 | 28: 33239306 |
| 10933 | 28: 33329500 | 10933 | 28: 33342708 | 10933 | 28: 33368759 | 10934 | 28: 33551708 | 10934 | 28: 33555527 |
| 10934 | 28: 33662006 | 10935 | 28: 33744728 | 10935 | 28: 33822791 | 10935 | 28: 33823084 | 10936 | 28: 34010065 |
| 10936 | 28: 34062194 | 10937 | 28: 34243304 | 10937 | 28: 34254734 | 10937 | 28: 34333090 | 10938 | 28: 34496870 |
| 10938 | 28: 34523399 | 10939 | 28: 34603788 | 10940 | 28: 34778574 | 10940 | 28: 34786048 | 10940 | 28: 34828202 |
| 10941 | 28: 34853555 | 10941 | 28: 34912784 | 10941 | 28: 34993324 | 10942 | 28: 35011293 | 10942 | 28: 35125823 |
| 10942 | 28: 35132199 | 10942 | 28: 35132247 | 10943 | 28: 35222450 | 10943 | 28: 35291319 | 10944 | 28: 35644657 |
| 10944 | 28: 35671648 | 10944 | 28: 35672175 | 10944 | 28: 35680732 | 10944 | 28: 35683234 | 10944 | 28: 35685429 |
| 10945 | 28: 35805891 | 10945 | 28: 35837667 | 10945 | 28: 35923596 | 10946 | 28: 36015548 | 10947 | 28: 36205983 |
| 10948 | 28: 36587691 | 10952 | 28: 37533725 | 10954 | 28: 37721207 | 10960 | 28: 39163650 | 10960 | 28: 39164865 |
| 10960 | 28: 39164902 | 10960 | 28: 39164949 | 10960 | 28: 39167717 | 10960 | 28: 39167797 | 10960 | 28: 39167807 |
| 10960 | 28: 39167808 | 10960 | 28: 39167836 | 10960 | 28: 39167853 | 10961 | 28: 39266326 | 10961 | 28: 39290663 |
| 10961 | 28: 39316523 | 10963 | 28: 39818069 | 10963 | 28: 39868243 | 10966 | 28: 40526877 | 10966 | 28: 40526886 |
| 10966 | 28: 40547129 | 10966 | 28: 40606521 | 10966 | 28: 40609071 | 10967 | 28: 40613418 | 10967 | 28: 40641661 |
| 10967 | 28: 40644608 | 10967 | 28: 40647540 | 10967 | 28: 40648502 | 10967 | 28: 40649183 | 10967 | 28: 40650128 |
| 10967 | 28: 40650866 | 10968 | 28: 40651548 | 10968 | 28: 40652228 | 10968 | 28: 40653623 | 10968 | 28: 40654155 |
| 10968 | 28: 40654919 | 10968 | 28: 40655651 | 10968 | 28: 40656367 | 10968 | 28: 40658031 | 10968 | 28: 40659095 |
| 10969 | 28: 40679353 | 10969 | 28: 40683789 | 10969 | 28: 40684338 | 10969 | 28: 40685176 | 10969 | 28: 40685911 |
| 10969 | 28: 40687468 | 10969 | 28: 40688010 | 10969 | 28: 40688917 | 10969 | 28: 40690412 | 10969 | 28: 40691166 |
| 10970 | 28: 40692798 | 10970 | 28: 40693384 | 10970 | 28: 40694350 | 10970 | 28: 40695394 | 10970 | 28: 40697087 |
| 10970 | 28: 40698875 | 10970 | 28: 40700189 | 10970 | 28: 40701565 | 10970 | 28: 40707053 | 10970 | 28: 40707560 |
| 10971 | 28: 40708330 | 10971 | 28: 40709943 | 10971 | 28: 40711617 | 10971 | 28: 40714408 | 10971 | 28: 40716186 |
| 10971 | 28: 40717895 | 10971 | 28: 40718746 | 10971 | 28: 40719687 | 10971 | 28: 40720636 | 10971 | 28: 40721303 |
| 10972 | 28: 40722936 | 10972 | 28: 40723874 | 10972 | 28: 40724503 | 10972 | 28: 40725696 | 10972 | 28: 40726977 |
| 10973 | 28: 40857491 | 10974 | 28: 40977297 | 10974 | 28: 41002895 | 10974 | 28: 41077744 | 10975 | 28: 41244211 |
| 10975 | 28: 41283427 | 10975 | 28: 41333995 | 10977 | 28: 41565674 | 10978 | 28: 41666097 | 10978 | 28: 41666186 |
| 10978 | 28: 41724320 | 10978 | 28: 41728249 | 10978 | 28: 41731568 | 10978 | 28: 41757290 | 10979 | 28: 41885934 |
| 10979 | 28: 41913803 | 10980 | 28: 41952391 | 10980 | 28: 41980728 | 10980 | 28: 41986845 | 10980 | 28: 41989663 |
| 10980 | 28: 42038658 | 10981 | 28: 42092356 | 10982 | 28: 42424892 | 10983 | 28: 42588414 | 10983 | 28: 42648031 |
| 10984 | 28: 42816073 | 10984 | 28: 42822333 | 10984 | 28: 42894677 | 10985 | 28: 43061595 | 10985 | 28: 43097491 |
| 10985 | 28: 43111417 | 10985 | 28: 43111489 | 10985 | 28: 43111570 | 10985 | 28: 43112699 | 10986 | 28: 43112752 |
| 10986 | 28: 43112807 | 10986 | 28: 43112817 | 10986 | 28: 43112858 | 10986 | 28: 43140781 | 10987 | 28: 43230125 |
| 10987 | 28: 43258213 | 10988 | 28: 43420442 | 10988 | 28: 43482124 | 10988 | 28: 43501640 | 10988 | 28: 43538406 |
| 10989 | 28: 43579200 | 10989 | 28: 43651140 | 10990 | 28: 44028372 | 10990 | 28: 44030407 | 10990 | 28: 44031081 |
| 10990 | 28: 44038721 | 10991 | 28: 44039225 | 10991 | 28: 44039304 | 10991 | 28: 44143344 | 10991 | 28: 44157666 |
| 10992 | 28: 44264765 | 10993 | 28: 44453518 | 10994 | 28: 44687207 | 10994 | 28: 44852965 | 10996 | 28: 45172059 |
| 10997 | 28: 45281713 | 10997 | 28: 45314101 | 10998 | 28: 45416826 | 10998 | 28: 45476974 | 10999 | 28: 45579104 |
| 11000 | 28: 45713679 | 11000 | 28: 45771532 | 11000 | 28: 45847568 | 11001 | 29: 612126 | 11001 | 29: 699249 |
| 11002 | 29: 699337 | 11002 | 29: 699732 | 11002 | 29: 699767 | 11002 | 29: 750365 | 11004 | 29: 1219671 |
| 11004 | 29: 1273559 | 11004 | 29: 1315459 | 11005 | 29: 1429261 | 11005 | 29: 1499682 | 11005 | 29: 1512567 |
| 11006 | 29: 1512626 | 11007 | 29: 1772440 | 11008 | 29: 2136150 | 11009 | 29: 2325537 | 11009 | 29: 2380491 |
| 11011 | 29: 2594653 | 11011 | 29: 2620380 | 11013 | 29: 2994930 | 11014 | 29: 3175555 | 11015 | 29: 3406656 |
| 11016 | 29: 3717232 | 11019 | 29: 4246237 | 11019 | 29: 4406319 | 11020 | 29: 4643160 | 11022 | 29: 5107639 |
| 11025 | 29: 6283155 | 11026 | 29: 6283208 | 11027 | 29: 6779650 | 11028 | 29: 7101326 | 11029 | 29: 7255100 |
| 11029 | 29: 7426428 | 11033 | 29: 8601758 | 11034 | 29: 8772433 | 11035 | 29: 8793187 | 11035 | 29: 8821458 |
| 11035 | 29: 8883493 | 11035 | 29: 8926820 | 11036 | 29: 8998775 | 11036 | 29: 9022834 | 11036 | 29: 9022891 |
| 11036 | 29: 9102521 | 11037 | 29: 9365821 | 11038 | 29: 9490114 | 11040 | 29: 9846120 | 11040 | 29: 9882247 |
| 11042 | 29: 10340072 | 11044 | 29: 10979545 | 11046 | 29: 11204386 | 11046 | 29: 11241638 | 11046 | 29: 11260132 |
| 11046 | 29: 11341144 | 11047 | 29: 11709562 | 11048 | 29: 11839933 | 11048 | 29: 11891707 | 11049 | 29: 11952953 |
| 11049 | 29: 11977550 | 11049 | 29: 12018235 | 11049 | 29: 12084416 | 11050 | 29: 12114717 | 11050 | 29: 12138482 |
| 11050 | 29: 12182249 | 11051 | 29: 12394797 | 11051 | 29: 12538136 | 11051 | 29: 12589589 | 11052 | 29: 12619588 |
| 11052 | 29: 12622700 | 11054 | 29: 12987411 | 11055 | 29: 13574420 | 11055 | 29: 13574513 | 11056 | 29: 13821500 |
| 11058 | 29: 14373492 | 11058 | 29: 14374640 | 11059 | 29: 14741850 | 11059 | 29: 14741861 | 11059 | 29: 14741873 |
| 11059 | 29: 14741887 | 11060 | 29: 14741921 | 11060 | 29: 14900262 | 11061 | 29: 15134872 | 11062 | 29: 15304699 |
| 11062 | 29: 15395863 | 11064 | 29: 15811607 | 11066 | 29: 16464456 | 11066 | 29: 16485296 | 11069 | 29: 16964102 |
| 11069 | 29: 16992973 | 11069 | 29: 17055985 | 11070 | 29: 17207473 | 11070 | 29: 17442223 | 11071 | 29: 17505934 |
| 11071 | 29: 17540650 | 11071 | 29: 17700379 | 11073 | 29: 18393448 | 11073 | 29: 18612768 | 11078 | 29: 20127489 |
| 11079 | 29: 20506894 | 11079 | 29: 20644568 | 11080 | 29: 20651157 | 11080 | 29: 20673987 | 11080 | 29: 20674048 |
| 11082 | 29: 21228889 | 11082 | 29: 21315170 | 11083 | 29: 21392121 | 11083 | 29: 21392184 | 11083 | 29: 21392189 |
| 11084 | 29: 21600071 | 11086 | 29: 22072294 | 11086 | 29: 22180877 | 11086 | 29: 22297264 | 11086 | 29: 22344163 |
| 11088 | 29: 22760861 | 11088 | 29: 22836272 | 11090 | 29: 23318559 | 11090 | 29: 23357907 | 11091 | 29: 23560620 |
| 11092 | 29: 23583982 | 11092 | 29: 23628287 | 11092 | 29: 23661992 | 11092 | 29: 23731784 | 11093 | 29: 23850587 |
| 11093 | 29: 23937388 | 11094 | 29: 24158412 | 11095 | 29: 24258248 | 11095 | 29: 24330084 | 11095 | 29: 24354233 |
| 11095 | 29: 24362811 | 11095 | 29: 24490616 | 11096 | 29: 24840874 | 11097 | 29: 25015460 | 11098 | 29: 25071999 |
| 11098 | 29: 25205531 | 11099 | 29: 25222326 | 11099 | 29: 25305438 | 11099 | 29: 25371765 | 11099 | 29: 25438251 |
| 11101 | 29: 25903150 | 11101 | 29: 25908734 | 11101 | 29: 26070069 | 11102 | 29: 26105651 | 11103 | 29: 26549594 |
| 11104 | 29: 26946250 | 11104 | 29: 26946336 | 11104 | 29: 26946383 | 11105 | 29: 26947201 | 11105 | 29: 26947224 |
| 11105 | 29: 26962161 | 11106 | 29: 27537716 | 11106 | 29: 27537760 | 11106 | 29: 27538148 | 11107 | 29: 27595444 |
| 11107 | 29: 27728132 | 11108 | 29: 27790794 | 11108 | 29: 27808076 | 11108 | 29: 27808486 | 11108 | 29: 27823998 |
| 11108 | 29: 27824016 | 11108 | 29: 27824039 | 11109 | 29: 27824126 | 11109 | 29: 28026650 | 11109 | 29: 28100144 |
| 11110 | 29: 28218734 | 11110 | 29: 28226254 | 11110 | 29: 28228229 | 11110 | 29: 28251283 | 111111 | 29: 28278699 |
| 111117 | 29: 28370448 | 111118 | 29: 28430575 | 11112 | 29: 28519839 | 11112 | 29: 28574787 | 11112 | 29: 28655873 |
| 11112 | 29: 28715263 | 11113 | 29: 28815692 | 11113 | 29: 28826292 | 11115 | 29: 29397513 | 11116 | 29: 29397515 |
| 11116 | 29: 29461701 | 11116 | 29: 29472010 | 11116 | 29: 29555287 | 11117 | 29: 29629619 | 11117 | 29: 29649318 |
| 11117 | 29: 29653990 | 11117 | 29: 29665600 | 11117 | 29: 29676297 | 11117 | 29: 29682536 | 11117 | 29: 29690811 |
| 11118 | 29: 29755215 | 11118 | 29: 29755216 | 11118 | 29: 29755261 | 11118 | 29: 29755320 | 11118 | 29: 29756679 |
| 11118 | 29: 29756692 | 11119 | 29: 29928478 | 11119 | 29: 29928524 | 11119 | 29: 29928553 | 11119 | 29: 29928591 |
| 11120 | 29: 29928636 | 11120 | 29: 29928675 | 11120 | 29: 29928677 | 11121 | 29: 30201851 | 11121 | 29: 30294652 |
| 11123 | 29: 30759911 | 11124 | 29: 30759984 | 11125 | 29: 31124828 | 11126 | 29: 31355454 | 11126 | 29: 31355522 |
| 11127 | 29: 31455230 | 11127 | 29: 31488540 | 11128 | 29: 31676141 | 11128 | 29: 31801460 | 11128 | 29: 31801542 |
| 11128 | 29: 31845002 | 11128 | 29: 31845960 | 11130 | 29: 32253717 | 11131 | 29: 32397724 | 11131 | 29: 32544942 |
| 11131 | 29: 32611173 | 11132 | 29: 32693113 | 11132 | 29: 32753972 | 11132 | 29: 32754754 | 11132 | 29: 32764982 |
| 11132 | 29: 32765663 | 11132 | 29: 32766346 | 11133 | 29: 32842956 | 11134 | 29: 33120665 | 11135 | 29: 33560766 |
| 11136 | 29: 33704537 | 11137 | 29: 33824948 | 11139 | 29: 34433856 | 11141 | 29: 34688201 | 11142 | 29: 34751704 |
| 11142 | 29: 34751748 | 11143 | 29: 34985663 | 11144 | 29: 35373548 | 11144 | 29: 35393862 | 11144 | 29: 35393931 |
| 11145 | 29: 35506238 | 11146 | 29: 35663595 | 11146 | 29: 35697578 | 11147 | 29: 35864204 | 11147 | 29: 35918743 |
| 11148 | 29: 36106383 | 11148 | 29: 36108259 | 11149 | 29: 36148048 | 11149 | 29: 36206784 | 11149 | 29: 36207683 |
| 11150 | 29: 36388797 | 11151 | 29: 36481048 | 11152 | 29: 36766544 | 11152 | 29: 36775708 | 11152 | 29: 36800395 |
| 11152 | 29: 36811950 | 11152 | 29: 36813596 | 11153 | 29: 36934958 | 11153 | 29: 36975121 | 11154 | 29: 37168472 |
| 11154 | 29: 37221849 | 11155 | 29: 37332886 | 11155 | 29: 37332893 | 11155 | 29: 37342276 | 11155 | 29: 37411101 |
| 11156 | 29: 37440660 | 11156 | 29: 37458594 | 11156 | 29: 37523548 | 11160 | 29: 39536492 | 11161 | 29: 39891282 |
| 11162 | 29: 39975025 | 11163 | 29: 40151594 | 11163 | 29: 40297458 | 11163 | 29: 40314843 | 11164 | 29: 40481400 |
| 11164 | 29: 40518208 | 11164 | 29: 40595649 | 11165 | 29: 40805372 | 11165 | 29: 40806225 | 11166 | 29: 40851566 |
| 11167 | 29: 40878959 | 11167 | 29: 40879481 | 11167 | 29: 40919327 | 11167 | 29: 41004847 | 11168 | 29: 41038278 |
| 11168 | 29: 41142315 | 11168 | 29: 41142793 | 11169 | 29: 41279012 | 11169 | 29: 41338346 | 11169 | 29: 41342454 |
| 11170 | 29: 41493354 | 11171 | 29: 41557548 | 11171 | 29: 41564691 | 11171 | 29: 41642963 | 11171 | 29: 41687163 |
| 11171 | 29: 41749646 | 11172 | 29: 41785049 | 11172 | 29: 41789280 | 11172 | 29: 41792341 | 11172 | 29: 41823721 |
| 11172 | 29: 41947165 | 11173 | 29: 41976753 | 11173 | 29: 42018005 | 11173 | 29: 42022273 | 11173 | 29: 42031453 |
| 11175 | 29: 42391692 | 11175 | 29: 42509147 | 11175 | 29: 42513501 | 11176 | 29: 42528204 | 11176 | 29: 42615699 |
| 11176 | 29: 42635388 | 11176 | 29: 42642354 | 11176 | 29: 42657038 | 11176 | 29: 42659255 | 11176 | 29: 42659448 |
| 11177 | 29: 42844516 | 11178 | 29: 42982094 | 11178 | 29: 43052218 | 11178 | 29: 43090186 | 11179 | 29: 43206957 |
| 11179 | 29: 43207132 | 11179 | 29: 43207525 | 11179 | 29: 43207573 | 11179 | 29: 43207753 | 11179 | 29: 43210991 |
| 11179 | 29: 43218240 | 11180 | 29: 43282578 | 11180 | 29: 43391770 | 11181 | 29: 43495617 | 11181 | 29: 43524321 |
| 11182 | 29: 43620118 | 11182 | 29: 43622532 | 11182 | 29: 43627877 | 11182 | 29: 43631768 | 11183 | 29: 43704875 |
| 11183 | 29: 43750816 | 11183 | 29: 43750820 | 11183 | 29: 43814690 | 11183 | 29: 43827364 | 11183 | 29: 43880340 |
| 11184 | 29: 43883371 | 11184 | 29: 43909067 | 11184 | 29: 43968492 | 11184 | 29: 43970000 | 11184 | 29: 43981948 |
| 11184 | 29: 43982210 | 11186 | 29: 44265806 | 11186 | 29: 44285086 | 11186 | 29: 44314568 | 11187 | 29: 44423551 |
| 11188 | 29: 44484826 | 11188 | 29: 44552650 | 11188 | 29: 44627679 | 11189 | 29: 44770084 | 11189 | 29: 44991893 |
| 11190 | 29: 45224759 | 11191 | 29: 45378882 | 11191 | 29: 45460407 | 11191 | 29: 45538531 | 11192 | 29: 45566486 |
| 11192 | 29: 45574701 | 11192 | 29: 45601782 | 11192 | 29: 45749221 | 11193 | 29: 45756539 | 11193 | 29: 45875877 |
| 11193 | 29: 45897665 | 11193 | 29: 45951010 | 11194 | 29: 45996529 | 11194 | 29: 46069944 | 11195 | 29: 46268948 |
| 11195 | 29: 46344185 | 11196 | 29: 46344338 | 11199 | 29: 47058184 | 11199 | 29: 47089599 | 11199 | 29: 47171895 |
| 11200 | 29: 47412530 | 11200 | 29: 47512321 | 11201 | 29: 47628422 | 11203 | 29: 47965964 | 11203 | 29: 48004279 |
| 11204 | 29: 48072601 | 11205 | 29: 48380729 | 11205 | 29: 48412845 | 11205 | 29: 48499387 | 11206 | 29: 48750418 |
| 11207 | 29: 48886024 | 11207 | 29: 48936775 | 11210 | 29: 49277470 | 11211 | 29: 49503212 | 11211 | 29: 49583754 |
| 11211 | 29: 49601539 | 11211 | 29: 49645960 | 11211 | 29: 49646022 | 11212 | 29: 49706299 | 11213 | 29: 50015783 |
| 11214 | 29: 50279949 | 11214 | 29: 50279990 | 11214 | 29: 50326170 | 11215 | 29: 50499209 | 11215 | 29: 50566734 |
| 11215 | 29: 50572585 | 11215 | 29: 50573514 | 11215 | 29: 50595441 | 11216 | 29: 50698048 | 11216 | 29: 50698051 |
| 11216 | 29: 50698125 | 11216 | 29: 50699876 | 11216 | 29: 50700624 | 11217 | 29: 50702620 | 11217 | 29: 50777521 |
| 11217 | 29: 50809985 | 11217 | 29: 50901915 | 11217 | 29: 50902670; | | | | |

and
the sites of the third marker panel are shown in the following table:

| NO. | Position | NO. | Position | NO. | Position | NO. | Position | NO. | Position |
|---|---|---|---|---|---|---|---|---|---|
| 188 | 1:395886 | 339 | 1:703722 | 420 | 1:906974 | 1171 | 1:2996340 | 1536 | 1:4057672 |
| 2248 | 1:589653 | 2403 | 1:626739 | 3852 | 1:985197 | 4392 | 1:1131760 | 4969 | 1:1264288 |
| 6050 | 1:149646 | 6468 | 1:156749 | 6762 | 2:537453 | 6787 | 2:5824469 | 6980 | 2:1042500 |
| 7049 | 2:128028 | 7090 | 2:138220 | 7681 | 2:269491 | 8255 | 2:4182328 | 8310 | 2:4316452 |
| 8401 | 2:457305 | 1090 | 2:110396 | 1119 | 2:117962 | 1166 | 2:1282694 | 1277 | 3:1267167 |
| 1313 | 3:211447 | 1403 | 3:395491 | 1406 | 3:402555 | 1410 | 3:4160522 | 1442 | 3:4954803 |
| 1453 | 3:518176 | 1475 | 3:578965 | 1529 | 3:734325 | 1573 | 3:8499077 | 1615 | 3:9391427 |
| 1628 | 3:975958 | 1723 | 3:115822 | 1798 | 4:109063 | 1818 | 4:1621515 | 1838 | 4:2065221 |
| 1897 | 4:367420 | 1915 | 4:414001 | 1971 | 4:574214 | 2019 | 4:7061032 | 2025 | 4:7253292 |
| 2047 | 4:779178 | 2197 | 4:109990 | 2288 | 5:765657 | 2371 | 5:2767099 | 2388 | 5:3169176 |
| 2505 | 5:629338 | 2520 | 5:666939 | 2543 | 5:719145 | 2568 | 5:7804644 | 2622 | 5:8792394 |
| 2671 | 5:976312 | 2750 | 5:112616 | 2783 | 5:118152 | 2851 | 6:1273217 | 2895 | 6:2221084 |
| 2919 | 6:281131 | 2919 | 6:281318 | 3226 | 6:429944 | 3250 | 6:4536462 | 3309 | 6:5955160 |
| 3311 | 6:598689 | 3355 | 6:728525 | 3507 | 6:109291 | 3527 | 6:1134849 | 3591 | 7:7248738 |
| 3605 | 7:121749 | 3634 | 7:172230 | 3671 | 7:248614 | 3790 | 7:5323337 | 3903 | 7:7930430 |
| 3904 | 7:793814 | 3910 | 7:805361 | 3961 | 7:918583 | 3970 | 7:9452918 | 4012 | 7:1009550 |
| 4031 | 7:105797 | 4055 | 8:168853 | 4167 | 8:287478 | 4284 | 8:5958533 | 4394 | 8:8752304 |
| 4449 | 8:101431 | 4466 | 8:104418 | 4553 | 9:142246 | 4608 | 9:2777847 | 4704 | 9:5170207 |
| 4729 | 9:585114 | 4832 | 9:822086 | 4908 | 9:970297 | 4966 | 10:358243 | 5021 | 10:146014 |
| 5033 | 10:16803 | 5143 | 10:44059 | 5146 | 10:44872 | 5192 | 10:555395 | 5212 | 10:614624 |
| 5294 | 10:81223 | 5300 | 10:82399 | 5307 | 10:84068 | 5367 | 10:971526 | 5448 | 11:940047 |
| 5509 | 11:24487 | 5558 | 11:36861 | 5596 | 11:46544 | 5660 | 11:620426 | 5663 | 11:627814 |
| 5678 | 11:66365 | 5735 | 11:77817 | 5818 | 11:95543 | 5851 | 11:102567 | 5855 | 11:103007 |
| 5918 | 12:11793 | 5977 | 12:25646 | 5999 | 12:31268 | 6090 | 12:578674 | 6099 | 12:609977 |
| 6124 | 12:68116 | 6155 | 12:75672 | 6156 | 12:76081 | 6217 | 13:207965 | 6317 | 13:253355 |
| 6422 | 13:47037 | 6513 | 13:68527 | 6542 | 13:74664 | 6584 | 13:826673 | 6638 | 14:911668 |
| 6643 | 14:10036 | 6757 | 14:26068 | 6840 | 14:46226 | 6866 | 14:534793 | 6896 | 14:602989 |
| 6969 | 14:77723 | 6977 | 14:79615 | 7089 | 15:20899 | 7148 | 15:329126 | 7170 | 15:374722 |
| 7170 | 15:37547 | 7194 | 15:42887 | 7229 | 15:49801 | 7231 | 15:506934 | 7297 | 15:638330 |
| 7359 | 15:77950 | 7425 | 16:92596 | 7433 | 16:11622 | 7440 | 16:130724 | 7460 | 16:191974 |
| 7675 | 16:71035 | 7696 | 16:75329 | 7727 | 17:10232 | 7766 | 17:913211 | 7781 | 17:127266 |
| 7801 | 17:17314 | 7846 | 17:29524 | 7847 | 17:29949 | 7853 | 17:314780 | 7929 | 17:504856 |
| 7946 | 17:54264 | 7998 | 17:64965 | 8050 | 18:18019 | 8074 | 18:674782 | 8150 | 18:233514 |
| 8157 | 18:24699 | 8166 | 18:26757 | 8251 | 18:46459 | 8268 | 18:485458 | 8283 | 18:521782 |
| 8345 | 18:64214 | 8371 | 19:44783 | 8389 | 19:82749 | 8403 | 19:108612 | 8423 | 19:150178 |
| 8432 | 19:16900 | 8553 | 19:38864 | 8558 | 19:40043 | 8579 | 19:441735 | 8624 | 19:522932 |
| 8636 | 19:54554 | 8673 | 19:59907 | 8708 | 20:23863 | 8795 | 20:178473 | 8854 | 20:309114 |
| 8917 | 20:46043 | 8962 | 20:58379 | 8983 | 20:63586 | 9034 | 21:304067 | 9114 | 21:208094 |
| 9134 | 21:26120 | 9139 | 21:27418 | 9192 | 21:39994 | 9205 | 21:431339 | 9241 | 21:516923 |
| 9288 | 21:60444 | 9301 | 21:63550 | 9383 | 22:11000 | 9386 | 22:117189 | 9424 | 22:209163 |
| 9428 | 22:21678 | 9431 | 22:22468 | 9440 | 22:25488 | 9546 | 22:505892 | 9559 | 22:534773 |
| 9573 | 22:55883 | 9635 | 23:73186 | 9740 | 23:27495 | 9815 | 23:409936 | 9820 | 23:420218 |
| 9868 | 23:51029 | 9883 | 24:16171 | 9903 | 24:61255 | 9928 | 24:104971 | 9946 | 24:151419 |
| 9970 | 24:21378 | 1006 | 24:44195 | 1011 | 24:55950 | 1017 | 25:313056 | 1023 | 25:145932 |
| 1025 | 25:19902 | 1028 | 25:27084 | 1028 | 25:27654 | 1040 | 26:819240 | 1042 | 26:131947 |
| 1052 | 26:37900 | 1053 | 26:39609 | 1057 | 26:49826 | 1062 | 27:920491 | 1063 | 27:117362 |
| 1064 | 27:13894 | 1065 | 27:16084 | 1067 | 27:19194 | 1068 | 27:224103 | 1074 | 27:357616 |
| 1075 | 27:37824 | 1077 | 27:41177 | 1081 | 28:58742 | 1085 | 28:159724 | 1087 | 28:203712 |
| 1093 | 28:33248 | 1094 | 28:35132 | 1099 | 28:43879 | 1103 | 29:910465 | 1104 | 29:111981 |
| 1111 | 29:28278 | 1111 | 29:28684 | 1118 | 29:44090 | 1121 | 29:508581; | | |

and
the sites of the fourth marker panel are shown in the following table:

| NO. | Position | NO. | Position | NO. | Position | NO. | Position | NO. | Position |
|---|---|---|---|---|---|---|---|---|---|
| 41 | 1:1299095 | 42 | 1:1301411 | 43 | 1:1302242 | 72 | 1:1675530 | 92 | 1:2080108 |
| 105 | 1:2256758 | 163 | 1:3595729 | 167 | 1:3620513 | 211 | 1:4298693 | 229 | 1:4729108 |
| 234 | 1:4821451 | 287 | 1:6049970 | 288 | 1:6052470 | 289 | 1:6054551 | 290 | 1:6056604 |
| 291 | 1:6061246 | 373 | 1:7795006 | 388 | 1:8249174 | 413 | 1:8847132 | 417 | 1:8982763 |
| 421 | 1:9088695 | 423 | 1:9118340 | 508 | 1:10838284 | 509 | 1:10876705 | 536 | 1:11604750 |
| 538 | 1:11752900 | 546 | 1:12052406 | 553 | 1:12362501 | 567 | 1:12653822 | 629 | 1:14114440 |
| 630 | 1:14176114 | 721 | 1:16635130 | 728 | 1:16922627 | 747 | 1:17462003 | 756 | 1:17835385 |
| 763 | 1:18110024 | 785 | 1:18793633 | 806 | 1:19543445 | 813 | 1:19664427 | 930 | 1:22893609 |
| 948 | 1:23486726 | 949 | 1:23493262 | 969 | 1:24006741 | 984 | 1:24500834 | 996 | 1:24941722 |
| 1001 | 1:25068708 | 1025 | 1:25577980 | 1129 | 1:29025677 | 1131 | 1:29040742 | 1225 | 1:31664947 |
| 1237 | 1:32296642 | 1256 | 1:32911987 | 1277 | 1:33660199 | 1286 | 1:33837966 | 1287 | 1:33843390 |
| 1288 | 1:33853447 | 1447 | 1:37890316 | 1452 | 1:38007855 | 1516 | 1:39993737 | 1545 | 1:40753557 |
| 1577 | 1:41764542 | 1622 | 1:42618603 | 1693 | 1:44281769 | 1695 | 1:44303304 | 1702 | 1:44457020 |
| 1705 | 1:44558674 | 1834 | 1:47759114 | 1879 | 1:49422581 | 1896 | 1:49969737 | 2024 | 1:53619617 |
| 2025 | 1:53623176 | 2026 | 1:53624697 | 2027 | 1:53629301 | 2028 | 1:53630113 | 2050 | 1:54016006 |
| 2075 | 1:54444238 | 2084 | 1:54690665 | 2116 | 1:55714510 | 2139 | 1:56372440 | 2335 | 1:61145631 |
| 2343 | 1:61319735 | 2362 | 1:61796684 | 2383 | 1:62427513 | 2384 | 1:62433558 | 2430 | 1:63221728 |
| 2462 | 1:64193131 | 2467 | 1:64262857 | 2472 | 1:64316563 | 2499 | 1:64907342 | 2526 | 1:65509479 |
| 2543 | 1:66070553 | 2602 | 1:67691264 | 2624 | 1:68339701 | 2675 | 1:69057373 | 2701 | 1:69645645 |
| 2739 | 1:70443699 | 2766 | 1:70688109 | 2800 | 1:71512292 | 2916 | 1:74460838 | 2934 | 1:74972802 |
| 2936 | 1:74999820 | 2946 | 1:75290882 | 2981 | 1:76075161 | 3007 | 1:76874012 | 3018 | 1:77236634 |
| 3022 | 1:77276581 | 3039 | 1:77623057 | 3041 | 1:77668859 | 3043 | 1:77697633 | 3046 | 1:77710839 |
| 3047 | 1:77723763 | 3060 | 1:78064459 | 3068 | 1:78315800 | 3079 | 1:78505641 | 3100 | 1:79138567 |
| 3115 | 1:79395243 | 3125 | 1:79597067 | 3165 | 1:80126772 | 3190 | 1:80929167 | 3251 | 1:82240750 |
| 3261 | 1:82501216 | 3275 | 1:82703570 | 3311 | 1:83985563 | 3319 | 1:84218035 | 3324 | 1:84478357 |
| 3325 | 1:84493668 | 3336 | 1:85023039 | 3352 | 1:85456698 | 3354 | 1:85501954 | 3382 | 1:86229426 |
| 3407 | 1:86980692 | 3460 | 1:88794416 | 3464 | 1:88824366 | 3545 | 1:90506530 | 3610 | 1:92190821 |
| 3612 | 1:92200875 | 3624 | 1:92563287 | 3638 | 1:93096031 | 3652 | 1:93351663 | 3654 | 1:93372952 |
| 3698 | 1:94573894 | 3724 | 1:95244942 | 3763 | 1:96215196 | 3768 | 1:96305965 | 3790 | 1:96826926 |
| 3791 | 1:96855345 | 3796 | 1:96968145 | 3806 | 1:97319365 | 3829 | 1:97847508 | 3860 | 1:98721777 |
| 3881 | 1:99293036 | 3929 | 1:100616517 | 3939 | 1:100987282 | 4172 | 1:108128123 | 4199 | 1:108777437 |
| 4225 | 1:109419276 | 4227 | 1:109445956 | 4261 | 1:110075319 | 4281 | 1:110389266 | 4305 | 1:111001754 |
| 4330 | 1:111772744 | 4338 | 1:111960165 | 4374 | 1:112577975 | 4386 | 1:112928362 | 4408 | 1:113414092 |
| 4421 | 1:113728857 | 4466 | 1:114828912 | 4467 | 1:114837165 | 4479 | 1:115162087 | 4480 | 1:115169020 |
| 4482 | 1:115183751 | 4521 | 1:116624187 | 4523 | 1:116668988 | 4524 | 1:116683980 | 4567 | 1:117562616 |
| 4604 | 1:118172592 | 4606 | 1:118199028 | 4607 | 1:118200955 | 4660 | 1:119155291 | 4714 | 1:120407128 |
| 4767 | 1:121845822 | 4796 | 1:122567824 | 4957 | 1:126246695 | 4964 | 1:126332119 | 5053 | 1:128692434 |
| 5062 | 1:128825902 | 5078 | 1:129263274 | 5234 | 1:132164555 | 5244 | 1:132550146 | 5263 | 1:133371805 |
| 5292 | 1:134238188 | 5295 | 1:134324146 | 5308 | 1:134489385 | 5335 | 1:135158239 | 5350 | 1:135504827 |
| 5358 | 1:135602604 | 5392 | 1:135798830 | 5450 | 1:137640097 | 5470 | 1:138610452 | 5474 | 1:138652323 |
| 5496 | 1:139117138 | 5556 | 1:140791857 | 5596 | 1:141481667 | 5678 | 1:142851690 | 5679 | 1:142863118 |
| 5697 | 1:143175205 | 5769 | 1:144589518 | 5777 | 1:144761021 | 5823 | 1:145661981 | 5912 | 1:147299699 |
| 5924 | 1:147514403 | 5943 | 1:147887268 | 5993 | 1:148725405 | 6105 | 1:150672403 | 6192 | 1:152095555 |
| 6198 | 1:152196690 | 6272 | 1:153106216 | 6277 | 1:153197360 | 6309 | 1:153828547 | 6340 | 1:154314444 |
| 6357 | 1:154855892 | 6360 | 1:154863975 | 6511 | 1:157806548 | 6568 | 2:1106317 | 6575 | 2:1169877 |
| 6578 | 2:1187683 | 6617 | 2:1860169 | 6621 | 2:1971521 | 6640 | 2:2590948 | 6666 | 2:3510323 |
| 6673 | 2:3709938 | 6718 | 2:4623152 | 6790 | 2:5906646 | 6800 | 2:6055644 | 6971 | 2:10245713 |
| 6979 | 2:10421191 | 7005 | 2:11387282 | 7060 | 2:13168802 | 7064 | 2:13243953 | 7176 | 2:16006718 |
| 7184 | 2:16189620 | 7214 | 2:16976649 | 7240 | 2:17639779 | 7274 | 2:18268568 | 7332 | 2:19056221 |
| 7365 | 2:19833213 | 7582 | 2:24849703 | 7630 | 2:25885564 | 7655 | 2:26463668 | 7685 | 2:27015449 |
| 7704 | 2:27459813 | 7705 | 2:27463924 | 7708 | 2:27525591 | 7760 | 2:28545094 | 7777 | 2:28922483 |
| 7807 | 2:29732106 | 7811 | 2:29813146 | 7889 | 2:31637856 | 7906 | 2:32169593 | 8046 | 2:36375875 |
| 8097 | 2:38039323 | 8098 | 2:38039898 | 8099 | 2:38041017 | 8100 | 2:38042315 | 8116 | 2:38297286 |
| 8120 | 2:38435225 | 8134 | 2:38750534 | 8153 | 2:39280801 | 8169 | 2:39667440 | 8177 | 2:39982069 |
| 8197 | 2:40615263 | 8225 | 2:41307524 | 8262 | 2:42027595 | 8270 | 2:42236387 | 8280 | 2:42439089 |
| 8281 | 2:42440990 | 8298 | 2:42711951 | 8329 | 2:43789161 | 8389 | 2:45494226 | 8399 | 2:45665663 |
| 8419 | 2:46116917 | 8473 | 2:47217427 | 8516 | 2:48495171 | 8517 | 2:48499243 | 8518 | 2:48502118 |
| 8519 | 2:48508248 | 8520 | 2:48511801 | 8560 | 2:49760042 | 8561 | 2:49760735 | 8611 | 2:51218510 |
| 8704 | 2:53518329 | 8730 | 2:54413562 | 8742 | 2:54672099 | 8762 | 2:55031934 | 8764 | 2:55069900 |
| 8765 | 2:55071242 | 8876 | 2:58381419 | 8884 | 2:58588299 | 8913 | 2:59316972 | 8960 | 2:60576956 |
| 8969 | 2:60779135 | 9031 | 2:62457322 | 9119 | 2:64653571 | 9135 | 2:64967881 | 9147 | 2:65185518 |
| 9201 | 2:66960608 | 9235 | 2:68024571 | 9384 | 2:72399848 | 9389 | 2:72514078 | 9418 | 2:73276155 |
| 9479 | 2:74974797 | 9480 | 2:74985065 | 9502 | 2:75739779 | 9535 | 2:76454912 | 9626 | 2:78834490 |
| 9641 | 2:79036468 | 9657 | 2:79510060 | 9681 | 2:80059990 | 9698 | 2:80586285 | 9747 | 2:83003800 |
| 9751 | 2:83109314 | 9755 | 2:83267596 | 9786 | 2:83877880 | 9795 | 2:84259043 | 9799 | 2:84375486 |
| 9832 | 2:85066770 | 9833 | 2:85070174 | 9851 | 2:85646050 | 9887 | 2:86425660 | 9898 | 2:86617347 |
| 9914 | 2:87174608 | 9921 | 2:87410708 | 9941 | 2:88035286 | 9972 | 2:88739478 | 10037 | 2:89511031 |
| 10038 | 2:89515068 | 10039 | 2:89518986 | 10040 | 2:89521529 | 10041 | 2:89523615 | 10076 | 2:90510385 |
| 10078 | 2:90533958 | 10114 | 2:91721645 | 10127 | 2:92066999 | 10128 | 2:92069312 | 10129 | 2:92085813 |
| 10130 | 2:92089545 | 10160 | 2:92756276 | 10181 | 2:93326093 | 10199 | 2:93469951 | 10200 | 2:93470548 |
| 10201 | 2:93471304 | 10202 | 2:93472560 | 10203 | 2:93473816 | 10218 | 2:93915068 | 10222 | 2:94006514 |
| 10223 | 2:94010942 | 10280 | 2:95335798 | 10338 | 2:96515244 | 10370 | 2:97455980 | 10377 | 2:97722574 |
| 10387 | 2:97882323 | 10401 | 2:98337087 | 10410 | 2:98432307 | 10413 | 2:98461459 | 10468 | 2:99951594 |
| 10492 | 2:100538824 | 10513 | 2:101147607 | 10555 | 2:102376439 | 10596 | 2:103531207 | 10650 | 2:104919346 |
| 10658 | 2:105027822 | 10663 | 2:105088054 | 10675 | 2:105412051 | 10829 | 2:108708703 | 10863 | 2:109439523 |
| 11027 | 2:113672892 | 11061 | 2:114526247 | 11202 | 2:118124674 | 11224 | 2:118587782 | 11225 | 2:118669657 |
| 11279 | 2:119878226 | 11336 | 2:121220519 | 11355 | 2:121820038 | 11356 | 2:121865885 | 11406 | 2:123297173 |
| 11455 | 2:124641406 | 11484 | 2:125479428 | 11617 | 2:127271059 | 11649 | 2:127896093 | 11743 | 2:130052119 |
| 11744 | 2:130058714 | 11745 | 2:130070890 | 11859 | 2:132478188 | 11860 | 2:132523448 | 11870 | 2:132734429 |
| 11917 | 2:133633752 | 11918 | 2:133637575 | 11939 | 2:133848234 | 11953 | 2:134015104 | 12115 | 3:525997 |
| 12118 | 3:556754 | 12143 | 3:950659 | 12145 | 3:954886 | 12153 | 3:1098608 | 12197 | 3:1960686 |
| 12261 | 3:3372085 | 12263 | 3:3434999 | 12280 | 3:3763587 | 12294 | 3:4101542 | 12315 | 3:4378889 |
| 12321 | 3:4437644 | 12343 | 3:4727841 | 12348 | 3:4795643 | 12369 | 3:5161089 | 12400 | 3:5776030 |
| 12410 | 3:6048242 | 12493 | 3:7480890 | 12527 | 3:7942262 | 12558 | 3:8433782 | 12561 | 3:8460244 |
| 12563 | 3:8520080 | 12564 | 3:8526638 | 12565 | 3:8529009 | 12566 | 3:8532372 | 12617 | 3:10020876 |
| 12620 | 3:10090524 | 12736 | 3:11477492 | 12754 | 3:11784186 | 12882 | 3:14796654 | 12934 | 3:15837312 |
| 13018 | 3:17753548 | 13048 | 3:18771436 | 13049 | 3:18775018 | 13050 | 3:18791656 | 13051 | 3:18795346 |
| 13056 | 3:18930543 | 13237 | 3:23451211 | 13314 | 3:25255575 | 13325 | 3:25405368 | 13343 | 3:25977712 |
| 13383 | 3:26543166 | 13431 | 3:27586116 | 13490 | 3:28659529 | 13578 | 3:30031426 | 13589 | 3:30407397 |
| 13632 | 3:31383515 | 13728 | 3:32928510 | 13731 | 3:32940407 | 13891 | 3:35285151 | 13909 | 3:35786483 |
| 13913 | 3:35908304 | 13922 | 3:36096832 | 13927 | 3:36253878 | 13990 | 3:37885414 | 14009 | 3:38624906 |
| 14010 | 3:38630748 | 14044 | 3:39845695 | 14058 | 3:40207827 | 14122 | 3:42050876 | 14139 | 3:42539907 |
| 14163 | 3:42995977 | 14232 | 3:44866434 | 14327 | 3:47649002 | 14398 | 3:49141481 | 14485 | 3:50111050 |
| 14521 | 3:51439192 | 14529 | 3:51625928 | 14618 | 3:53775526 | 14654 | 3:55087957 | 14667 | 3:55359809 |
| 14686 | 3:56006266 | 14688 | 3:56067950 | 14793 | 3:59053277 | 14797 | 3:59211837 | 14806 | 3:59443494 |
| 14818 | 3:59847747 | 14845 | 3:60575455 | 14847 | 3:60611151 | 14982 | 3:64188398 | 15009 | 3:65159985 |
| 15035 | 3:65721928 | 15088 | 3:66944634 | 15124 | 3:67769302 | 15169 | 3:68874408 | 15180 | 3:69234087 |
| 15187 | 3:69431230 | 15225 | 3:70347328 | 15264 | 3:72041156 | 15338 | 3:74664014 | 15421 | 3:77158063 |
| 15495 | 3:79128870 | 15545 | 3:80676731 | 15627 | 3:82625118 | 15652 | 3:83549724 | 15659 | 3:83658003 |
| 15683 | 3:84227201 | 15750 | 3:85215781 | 15768 | 3:85766170 | 15778 | 3:85960283 | 15779 | 3:85964163 |
| 15780 | 3:85967104 | 15781 | 3:85968892 | 15782 | 3:85969726 | 15798 | 3:86300913 | 15807 | 3:86441083 |
| 15908 | 3:88661891 | 15916 | 3:88782949 | 15917 | 3:88784034 | 15935 | 3:89220299 | 15977 | 3:89929116 |
| 16037 | 3:91288695 | 16170 | 3:94375264 | 16194 | 3:95152254 | 16204 | 3:95487322 | 16235 | 3:96416999 |
| 16276 | 3:97475924 | 16328 | 3:98728026 | 16330 | 3:98766843 | 16366 | 3:99717720 | 16400 | 3:100301019 |
| 16439 | 3:100971847 | 16445 | 3:101130049 | 16459 | 3:101339407 | 16485 | 3:101926220 | 16537 | 3:102883747 |
| 16572 | 3:103741473 | 16602 | 3:104484207 | 16605 | 3:104506752 | 16620 | 3:104710861 | 16639 | 3:105175424 |
| 16646 | 3:105209686 | 16684 | 3:105603312 | 16714 | 3:105965567 | 16715 | 3:105973059 | 16740 | 3:106492826 |
| 16774 | 3:107138615 | 16811 | 3:107842370 | 16874 | 3:109092493 | 16881 | 3:109399680 | 16887 | 3:109428951 |
| 16913 | 3:109808282 | 16959 | 3:110964139 | 17043 | 3:112687905 | 17134 | 3:114337662 | 17162 | 3:114723123 |
| 17166 | 3:114787475 | 17167 | 3:114787630 | 17168 | 3:114788049 | 17169 | 3:114788632 | 17170 | 3:114789131 |
| 17172 | 3:114791529 | 17173 | 3:114799492 | 17176 | 3:114803442 | 17177 | 3:114803451 | 17178 | 3:114803465 |
| 17179 | 3:114804161 | 17180 | 3:114804367 | 17195 | 3:115056511 | 17196 | 3:115071383 | 17206 | 3:115299586 |
| 17211 | 3:115381373 | 17407 | 3:118290616 | 17448 | 3:118879237 | 17676 | 4:4683962 | 17696 | 4:5003827 |
| 17732 | 4:5560351 | 17733 | 4:5568155 | 17760 | 4:6327312 | 17789 | 4:7010182 | 17804 | 4:7324916 |
| 17806 | 4:7326126 | 17855 | 4:8442378 | 17985 | 4:10842025 | 18045 | 4:12514592 | 18143 | 4:15186933 |
| 18155 | 4:15475039 | 18161 | 4:15588873 | 18162 | 4:15592728 | 18180 | 4:16210509 | 18257 | 4:18112247 |
| 18263 | 4:18273472 | 18273 | 4:18595000 | 18299 | 4:18985118 | 18400 | 4:20821405 | 18441 | 4:21738699 |
| 18444 | 4:21789346 | 18448 | 4:21942101 | 18464 | 4:22381610 | 18492 | 4:22930826 | 18520 | 4:23861032 |
| 18617 | 4:26517471 | 18748 | 4:30365149 | 18770 | 4:30805170 | 18813 | 4:31905587 | 18817 | 4:31972387 |
| 18879 | 4:33771936 | 18945 | 4:35882843 | 19025 | 4:38124757 | 19039 | 4:38555013 | 19086 | 4:40002914 |
| 19087 | 4:40007566 | 19088 | 4:40012097 | 19089 | 4:40018785 | 19090 | 4:40021130 | 19091 | 4:40041810 |
| 19092 | 4:40049842 | 19093 | 4:40054177 | 19094 | 4:40059057 | 19095 | 4:40060306 | 19115 | 4:40634211 |
| 19116 | 4:40640519 | 19117 | 4:40645360 | 19118 | 4:40649968 | 19119 | 4:40654713 | 19167 | 4:41814723 |
| 19172 | 4:42014141 | 19183 | 4:42356867 | 19190 | 4:42554917 | 19255 | 4:44244202 | 19291 | 4:45415922 |
| 19296 | 4:45536174 | 19308 | 4:45825175 | 19335 | 4:46718903 | 19342 | 4:46848225 | 19357 | 4:47216690 |
| 19358 | 4:47217662 | 19359 | 4:47221067 | 19360 | 4:47224473 | 19361 | 4:47228083 | 19400 | 4:48100380 |
| 19463 | 4:49408307 | 19575 | 4:53093575 | 19600 | 4:53722558 | 19607 | 4:53807295 | 19720 | 4:57434269 |
| 19763 | 4:58913134 | 19783 | 4:59391253 | 19787 | 4:59489126 | 19813 | 4:60378132 | 19818 | 4:60482892 |
| 19853 | 4:61414628 | 19854 | 4:61423853 | 19855 | 4:61432230 | 19856 | 4:61434936 | 19857 | 4:61439997 |
| 19881 | 4:62128232 | 19885 | 4:62171821 | 19916 | 4:63134475 | 19951 | 4:63940606 | 19960 | 4:64270176 |
| 19961 | 4:64271209 | 19964 | 4:64495002 | 19995 | 4:65572773 | 20043 | 4:66416290 | 20063 | 4:67092276 |
| 20082 | 4:67523624 | 20114 | 4:68300881 | 20190 | 4:70507936 | 20193 | 4:70569760 | 20246 | 4:72066682 |
| 20297 | 4:73725730 | 20302 | 4:73904776 | 20343 | 4:74843205 | 20361 | 4:75190744 | 20382 | 4:75792193 |
| 20390 | 4:75908626 | 20442 | 4:77149758 | 20462 | 4:77552330 | 20482 | 4:77954957 | 20489 | 4:78153455 |
| 20519 | 4:79093225 | 20523 | 4:79236024 | 20527 | 4:79350296 | 20537 | 4:79637506 | 20607 | 4:81119261 |
| 20687 | 4:83090213 | 20854 | 4:87615934 | 20860 | 4:87832774 | 20862 | 4:87887042 | 20866 | 4:87931932 |
| 20867 | 4:87937433 | 20918 | 4:89596848 | 20927 | 4:89756186 | 20933 | 4:89854090 | 20952 | 4:90405270 |
| 20965 | 4:90752463 | 20983 | 4:91186390 | 21013 | 4:91662043 | 21020 | 4:91835951 | 21026 | 4:91985689 |
| 21064 | 4:92683225 | 21143 | 4:94586866 | 21162 | 4:95121580 | 21208 | 4:95976133 | 21209 | 4:95978012 |
| 21210 | 4:95984102 | 21211 | 4:95986472 | 21212 | 4:95988223 | 21213 | 4:95991905 | 21214 | 4:95994577 |
| 21215 | 4:95996626 | 21216 | 4:95999064 | 21217 | 4:95999599 | 21218 | 4:95999657 | 21220 | 4:96006244 |
| 21223 | 4:96012342 | 21224 | 4:96014135 | 21225 | 4:96014452 | 21226 | 4:96014495 | 21229 | 4:96043384 |
| 21230 | 4:96043881 | 21258 | 4:96806394 | 21259 | 4:96808296 | 21260 | 4:96808305 | 21261 | 4:96808793 |
| 21263 | 4:96810164 | 21264 | 4:96810688 | 21265 | 4:96810739 | 21266 | 4:96811491 | 21267 | 4:96811492 |
| 21268 | 4:96811663 | 21269 | 4:96819713 | 21270 | 4:96819807 | 21271 | 4:96819821 | 21272 | 4:96820983 |
| 21273 | 4:96821494 | 21274 | 4:96822544 | 21275 | 4:96822709 | 21276 | 4:96822722 | 21277 | 4:96823395 |
| 21278 | 4:96823570 | 21280 | 4:96826910 | 21281 | 4:96827344 | 21282 | 4:96828046 | 21283 | 4:96828753 |
| 21284 | 4:96829524 | 21286 | 4:96842438 | 21287 | 4:96842474 | 21288 | 4:96846828 | 21289 | 4:96847175 |
| 21290 | 4:96847270 | 21291 | 4:96847298 | 21292 | 4:96847522 | 21293 | 4:96847685 | 21294 | 4:96848163 |
| 21295 | 4:96852063 | 21296 | 4:96852112 | 21297 | 4:96852128 | 21299 | 4:96853114 | 21300 | 4:96853149 |
| 21301 | 4:96854041 | 21302 | 4:96854052 | 21303 | 4:96854217 | 21304 | 4:96854309 | 21305 | 4:96854674 |
| 21306 | 4:96854753 | 21307 | 4:96854840 | 21308 | 4:96854846 | 21309 | 4:96855073 | 21310 | 4:96855142 |
| 21311 | 4:96855211 | 21312 | 4:96855234 | 21313 | 4:96855382 | 21314 | 4:96858356 | 21315 | 4:96858411 |
| 21316 | 4:96858620 | 21317 | 4:96859305 | 21318 | 4:96859677 | 21319 | 4:96859857 | 21320 | 4:96860289 |
| 21321 | 4:96860821 | 21322 | 4:96861792 | 21323 | 4:96862653 | 21324 | 4:96868903 | 21325 | 4:96870132 |
| 21326 | 4:96870621 | 21327 | 4:96871036 | 21328 | 4:96871329 | 21329 | 4:96872714 | 21330 | 4:96872734 |
| 21331 | 4:96878187 | 21332 | 4:96878242 | 21333 | 4:96878278 | 21334 | 4:96880890 | 21335 | 4:96880962 |
| 21336 | 4:96881026 | 21337 | 4:96881161 | 21338 | 4:96881966 | 21339 | 4:96882244 | 21340 | 4:96885170 |
| 21341 | 4:96886853 | 21342 | 4:96887160 | 21343 | 4:96887442 | 21344 | 4:96887486 | 21345 | 4:96887810 |
| 21346 | 4:96888595 | 21347 | 4:96889148 | 21348 | 4:96890359 | 21349 | 4:96890450 | 21350 | 4:96890701 |
| 21351 | 4:96891069 | 21352 | 4:96891335 | 21353 | 4:96891542 | 21354 | 4:96891552 | 21355 | 4:96891608 |
| 21356 | 4:96895620 | 21357 | 4:96897050 | 21358 | 4:96897145 | 21367 | 4:97039076 | 21370 | 4:97088268 |
| 21372 | 4:97137855 | 21373 | 4:97147752 | 21374 | 4:97148005 | 21375 | 4:97148878 | 21376 | 4:97150124 |
| 21377 | 4:97150300 | 21378 | 4:97151421 | 21379 | 4:97151944 | 21380 | 4:97152001 | 21381 | 4:97152026 |
| 21382 | 4:97152080 | 21383 | 4:97152151 | 21384 | 4:97152177 | 21385 | 4:97152300 | 21386 | 4:97152327 |
| 21387 | 4:97152343 | 21388 | 4:97152464 | 21389 | 4:97152562 | 21390 | 4:97152880 | 21391 | 4:97153058 |
| 21392 | 4:97160379 | 21393 | 4:97160479 | 21394 | 4:97160507 | 21395 | 4:97160725 | 21396 | 4:97160764 |
| 21397 | 4:97160849 | 21398 | 4:97160967 | 21399 | 4:97161387 | 21400 | 4:97161554 | 21401 | 4:97161881 |
| 21402 | 4:97161958 | 21404 | 4:97164436 | 21405 | 4:97165222 | 21406 | 4:97165446 | 21407 | 4:97165671 |
| 21408 | 4:97165867 | 21409 | 4:97167273 | 21410 | 4:97170350 | 21411 | 4:97170722 | 21412 | 4:97170758 |
| 21413 | 4:97170786 | 21415 | 4:97171793 | 21417 | 4:97173637 | 21418 | 4:97173882 | 21420 | 4:97243175 |
| 21429 | 4:97478233 | 21449 | 4:97997496 | 21460 | 4:98301073 | 21488 | 4:99227319 | 21496 | 4:99551602 |
| 21525 | 4:100096858 | 21571 | 4:101147322 | 21621 | 4:102291227 | 21665 | 4:103373158 | 21709 | 4:104494825 |
| 21712 | 4:104541226 | 21740 | 4:105243840 | 21826 | 4:106811124 | 21828 | 4:106840758 | 21903 | 4:108136633 |
| 21927 | 4:108959151 | 21976 | 4:110025220 | 22029 | 4:111137956 | 22031 | 4:111174901 | 22032 | 4:111185441 |
| 22052 | 4:111594642 | 22057 | 4:111656507 | 22081 | 4:112008507 | 22182 | 4:113533731 | 22254 | 4:114468144 |
| 22334 | 4:115504063 | 22339 | 4:115674188 | 22348 | 4:115903138 | 22484 | 4:118639815 | 22639 | 5:1916328 |
| 22664 | 5:2361885 | 22679 | 5:2678940 | 22680 | 5:2710438 | 22682 | 5:2775410 | 22683 | 5:2789169 |
| 22692 | 5:3084344 | 22721 | 5:4106293 | 22747 | 5:4642231 | 22801 | 5:5757045 | 22812 | 5:5962601 |
| 22864 | 5:7150201 | 22875 | 5:7320087 | 22901 | 5:8063807 | 22907 | 5:8295061 | 22914 | 5:8547280 |
| 23001 | 5:10417113 | 23005 | 5:10483003 | 23116 | 5:12929697 | 23147 | 5:13814938 | 23154 | 5:14118702 |
| 23200 | 5:15181434 | 23209 | 5:15382347 | 23335 | 5:18879061 | 23345 | 5:19180052 | 23365 | 5:19703644 |
| 23420 | 5:21141526 | 23424 | 5:21247771 | 23441 | 5:21652414 | 23488 | 5:22524662 | 23553 | 5:23856635 |
| 23577 | 5:24327813 | 23580 | 5:24363604 | 23609 | 5:25025028 | 23638 | 5:25910383 | 23667 | 5:26553269 |
| 23765 | 5:28658495 | 23768 | 5:28689345 | 23795 | 5:29481439 | 23827 | 5:30068971 | 23860 | 5:30880544 |
| 23885 | 5:31649328 | 23903 | 5:32253508 | 23967 | 5:33731249 | 23969 | 5:33743644 | 23990 | 5:34527357 |
| 24023 | 5:35215343 | 24041 | 5:35917350 | 24064 | 5:36532219 | 24164 | 5:39219011 | 24213 | 5:40323694 |
| 24252 | 5:41581614 | 24255 | 5:41737754 | 24277 | 5:42488185 | 24295 | 5:42883310 | 24309 | 5:43239107 |
| 24321 | 5:43556568 | 24332 | 5:43826757 | 24373 | 5:44749275 | 24394 | 5:45422250 | 24412 | 5:45892148 |
| 24469 | 5:47071100 | 24474 | 5:47204283 | 24476 | 5:47210190 | 24491 | 5:47544371 | 24515 | 5:48255910 |
| 24533 | 5:48697507 | 24700 | 5:53392371 | 24725 | 5:54050541 | 24767 | 5:54925075 | 24868 | 5:57315947 |
| 24869 | 5:57316513 | 24870 | 5:57318993 | 24931 | 5:59393649 | 25012 | 5:61592328 | 25049 | 5:62915639 |
| 25065 | 5:63217741 | 25070 | 5:63272705 | 25094 | 5:63826831 | 25189 | 5:66342506 | 25206 | 5:66729461 |
| 25248 | 5:67796979 | 25274 | 5:68252711 | 25308 | 5:69044504 | 25336 | 5:69658212 | 25386 | 5:70977990 |
| 25396 | 5:71157250 | 25405 | 5:71284786 | 25414 | 5:71441252 | 25415 | 5:71451984 | 25452 | 5:72240212 |
| 25455 | 5:72267018 | 25501 | 5:73577352 | 25505 | 5:73676579 | 25512 | 5:73764801 | 25516 | 5:73813825 |
| 25528 | 5:74101395 | 25540 | 5:74482047 | 25592 | 5:75448318 | 25633 | 5:76668839 | 25639 | 5:76766433 |
| 25644 | 5:76890153 | 25683 | 5:77996585 | 25684 | 5:78020028 | 25716 | 5:78821705 | 25736 | 5:79345129 |
| 25744 | 5:79644695 | 25768 | 5:80587434 | 25796 | 5:81407773 | 25800 | 5:81437991 | 25801 | 5:81441963 |
| 25851 | 5:82570119 | 25855 | 5:82593018 | 25856 | 5:82593228 | 25857 | 5:82593982 | 25858 | 5:82594019 |
| 25859 | 5:82594028 | 25860 | 5:82594375 | 25861 | 5:82594380 | 25862 | 5:82595065 | 25863 | 5:82595148 |
| 25864 | 5:82595334 | 25865 | 5:82627761 | 25905 | 5:83646235 | 26064 | 5:84298547 | 26072 | 5:84433271 |
| 26074 | 5:84527341 | 26089 | 5:84948836 | 26145 | 5:86376332 | 26148 | 5:86455776 | 26196 | 5:87435829 |
| 26293 | 5:88856288 | 26361 | 5:90566853 | 26403 | 5:91501158 | 26409 | 5:91665913 | 26415 | 5:91761326 |
| 26432 | 5:92332808 | 26496 | 5:93178439 | 26526 | 5:93790346 | 26533 | 5:93961735 | 26537 | 5:94088171 |
| 26579 | 5:95091626 | 26625 | 5:96054240 | 26685 | 5:97071869 | 26781 | 5:98982241 | 26878 | 5:100843102 |
| 26891 | 5:101049907 | 26948 | 5:102626302 | 27117 | 5:105882827 | 27183 | 5:106801714 | 27187 | 5:106877986 |
| 27206 | 5:107060379 | 27244 | 5:107745197 | 27305 | 5:108794780 | 27306 | 5:108797633 | 27341 | 5:109446738 |
| 27366 | 5:109926959 | 27428 | 5:111036587 | 27579 | 5:114683035 | 27584 | 5:114814526 | 27611 | 5:115497329 |
| 27613 | 5:115504227 | 27623 | 5:115614570 | 27664 | 5:116449909 | 27776 | 5:117284799 | 27782 | 5:117411822 |
| 27811 | 5:117753977 | 27824 | 5:117977735 | 27839 | 5:118288125 | 27856 | 5:118666844 | 27917 | 5:119642118 |
| 27951 | 6:579203 | 28082 | 6:3248784 | 28098 | 6:3406400 | 28122 | 6:3655995 | 28164 | 6:4606383 |
| 28201 | 6:5673122 | 28210 | 6:5813338 | 28269 | 6:6797112 | 28271 | 6:6808370 | 28384 | 6:9740976 |
| 28388 | 6:9896356 | 28418 | 6:10695206 | 28487 | 6:12244147 | 28494 | 6:12408823 | 28501 | 6:12515175 |
| 28516 | 6:12899581 | 28534 | 6:13157939 | 28535 | 6:13158398 | 28536 | 6:13158702 | 28570 | 6:14149976 |
| 28645 | 6:15766628 | 28649 | 6:15875705 | 28664 | 6:16165237 | 28720 | 6:17350197 | 28729 | 6:17546682 |
| 28775 | 6:18247972 | 28798 | 6:18858161 | 28832 | 6:19517669 | 28855 | 6:20067345 | 28903 | 6:21187469 |
| 28951 | 6:22198236 | 28974 | 6:22627641 | 28985 | 6:22838005 | 28994 | 6:23185445 | 29012 | 6:23551846 |
| 29033 | 6:24079559 | 29091 | 6:25420968 | 29092 | 6:25430886 | 29113 | 6:25987389 | 29124 | 6:26122133 |
| 29169 | 6:27357262 | 29187 | 6:27922767 | 29228 | 6:29044156 | 29256 | 6:29526067 | 29268 | 6:29755371 |
| 29360 | 6:30504972 | 29389 | 6:31372994 | 29391 | 6:31402252 | 29427 | 6:32495711 | 29446 | 6:33106068 |
| 29505 | 6:34316541 | 29908 | 6:34880169 | 29988 | 6:35895370 | 29990 | 6:35897546 | 29991 | 6:35897939 |
| 29992 | 6:35906185 | 29993 | 6:35907046 | 29995 | 6:35909397 | 29998 | 6:35925489 | 30025 | 6:36097722 |
| 30135 | 6:36427342 | 30137 | 6:36430013 | 30140 | 6:36432356 | 30143 | 6:36441375 | 30257 | 6:36563183 |
| 30260 | 6:36568261 | 30264 | 6:36572899 | 30333 | 6:36813124 | 30455 | 6:37026863 | 30456 | 6:37027429 |
| 30457 | 6:37027539 | 30458 | 6:37028623 | 30460 | 6:37030480 | 30461 | 6:37032906 | 30462 | 6:37032995 |
| 30467 | 6:37035186 | 30469 | 6:37038017 | 30471 | 6:37056413 | 30475 | 6:37075910 | 30566 | 6:37095299 |
| 30616 | 6:37107589 | 30626 | 6:37117771 | 30629 | 6:37121529 | 30630 | 6:37123023 | 30632 | 6:37130785 |
| 30633 | 6:37134418 | 30636 | 6:37136794 | 30643 | 6:37142174 | 30649 | 6:37149945 | 30685 | 6:37161408 |
| 30736 | 6:37173048 | 30788 | 6:37184577 | 30791 | 6:37188623 | 30792 | 6:37189736 | 30798 | 6:37206928 |
| 30799 | 6:37210690 | 30802 | 6:37214389 | 30803 | 6:37214736 | 30810 | 6:37219609 | 30813 | 6:37221776 |
| 30818 | 6:37227250 | 30820 | 6:37231623 | 30829 | 6:37235373 | 30830 | 6:37235374 | 30831 | 6:37236226 |
| 30844 | 6:37244478 | 30849 | 6:37247874 | 30851 | 6:37250850 | 30852 | 6:37251132 | 30854 | 6:37252978 |
| 30856 | 6:37256488 | 30857 | 6:37260221 | 30858 | 6:37262825 | 30859 | 6:37262995 | 30864 | 6:37269061 |
| 30865 | 6:37270215 | 30866 | 6:37270930 | 30867 | 6:37271635 | 30870 | 6:37281308 | 30871 | 6:37281335 |
| 30872 | 6:37282356 | 30873 | 6:37284874 | 30879 | 6:37286627 | 30880 | 6:37286722 | 30881 | 6:37287306 |
| 30882 | 6:37287423 | 30883 | 6:37287873 | 30884 | 6:37288219 | 30885 | 6:37288483 | 30886 | 6:37288600 |
| 30887 | 6:37288948 | 30888 | 6:37289071 | 30889 | 6:37289572 | 30890 | 6:37289742 | 30891 | 6:37289905 |
| 30892 | 6:37289990 | 30893 | 6:37290180 | 30894 | 6:37290241 | 30895 | 6:37290415 | 30896 | 6:37291360 |
| 30897 | 6:37291500 | 30898 | 6:37292117 | 30899 | 6:37292269 | 30900 | 6:37292270 | 30901 | 6:37292298 |
| 30902 | 6:37292752 | 30903 | 6:37293476 | 30905 | 6:37294505 | 30913 | 6:37297426 | 30917 | 6:37298725 |
| 30927 | 6:37300810 | 30932 | 6:37301959 | 30936 | 6:37302499 | 30938 | 6:37303255 | 30946 | 6:37304513 |
| 30952 | 6:37305466 | 30956 | 6:37306944 | 30959 | 6:37309266 | 30960 | 6:37310413 | 30972 | 6:37313503 |
| 30977 | 6:37314183 | 30993 | 6:37317695 | 31003 | 6:37321049 | 31004 | 6:37321441 | 31010 | 6:37324718 |
| 31018 | 6:37328528 | 31026 | 6:37329878 | 31030 | 6:37330306 | 31032 | 6:37330477 | 31033 | 6:37330577 |
| 31036 | 6:37331712 | 31046 | 6:37335926 | 31052 | 6:37337744 | 31056 | 6:37340937 | 31058 | 6:37342568 |
| 31066 | 6:37345508 | 31070 | 6:37347130 | 31071 | 6:37349144 | 31075 | 6:37351767 | 31076 | 6:37352145 |
| 31086 | 6:37359765 | 31088 | 6:37360685 | 31089 | 6:37361449 | 31090 | 6:37361578 | 31091 | 6:37362262 |
| 31099 | 6:37369338 | 31100 | 6:37369380 | 31102 | 6:37372334 | 31108 | 6:37376289 | 31111 | 6:37378475 |
| 31113 | 6:37379506 | 31114 | 6:37379507 | 31118 | 6:37380161 | 31120 | 6:37381106 | 31122 | 6:37381782 |
| 31123 | 6:37382003 | 31124 | 6:37382062 | 31127 | 6:37383526 | 31129 | 6:37385973 | 31133 | 6:37391915 |
| 31138 | 6:37393949 | 31139 | 6:37394877 | 31140 | 6:37395313 | 31143 | 6:37396789 | 31144 | 6:37398199 |
| 31145 | 6:37399560 | 31147 | 6:37400741 | 31151 | 6:37403131 | 31152 | 6:37403224 | 31153 | 6:37406239 |
| 31154 | 6:37406485 | 31156 | 6:37407653 | 31157 | 6:37412062 | 31162 | 6:37415367 | 31163 | 6:37416259 |
| 31166 | 6:37417501 | 31167 | 6:37418164 | 31169 | 6:37418447 | 31170 | 6:37418449 | 31172 | 6:37420957 |
| 31174 | 6:37424248 | 31176 | 6:37429071 | 31178 | 6:37429776 | 31179 | 6:37430447 | 31180 | 6:37431101 |
| 31181 | 6:37432458 | 31186 | 6:37435861 | 31188 | 6:37436347 | 31191 | 6:37437538 | 31192 | 6:37438222 |
| 31197 | 6:37438993 | 31199 | 6:37440571 | 31204 | 6:37444900 | 31208 | 6:37449890 | 31212 | 6:37457249 |
| 31216 | 6:37465669 | 31217 | 6:37466194 | 31218 | 6:37473453 | 31219 | 6:37477629 | 31221 | 6:37480277 |
| 31222 | 6:37482054 | 31225 | 6:37491305 | 31229 | 6:37503406 | 31230 | 6:37505093 | 31231 | 6:37508184 |
| 31235 | 6:37513645 | 31239 | 6:37522580 | 31240 | 6:37526812 | 31243 | 6:37534297 | 31246 | 6:37553481 |
| 31248 | 6:37560625 | 31249 | 6:37562383 | 31251 | 6:37566268 | 31252 | 6:37566317 | 31254 | 6:37573307 |
| 31255 | 6:37573615 | 31256 | 6:37578832 | 31262 | 6:37587980 | 31263 | 6:37588492 | 31264 | 6:37591326 |
| 31265 | 6:37591664 | 31268 | 6:37598284 | 31276 | 6:37608504 | 31278 | 6:37614179 | 31280 | 6:37624003 |
| 31281 | 6:37625923 | 31282 | 6:37627776 | 31284 | 6:37636883 | 31285 | 6:37636900 | 31286 | 6:37637484 |
| 31287 | 6:37639651 | 31288 | 6:37639763 | 31289 | 6:37640954 | 31290 | 6:37641448 | 31291 | 6:37641798 |
| 31292 | 6:37642775 | 31293 | 6:37644691 | 31294 | 6:37644915 | 31295 | 6:37645671 | 31296 | 6:37646000 |
| 31297 | 6:37646499 | 31301 | 6:37658848 | 31349 | 6:37772675 | 31353 | 6:37774778 | 31355 | 6:37775624 |
| 31357 | 6:37776401 | 31359 | 6:37776454 | 31360 | 6:37776464 | 31361 | 6:37777029 | 31363 | 6:37777185 |
| 31404 | 6:37817575 | 31438 | 6:37912116 | 31445 | 6:37914472 | 31454 | 6:37917366 | 31455 | 6:37917421 |
| 31465 | 6:37919853 | 31475 | 6:37926073 | 31476 | 6:37926600 | 31477 | 6:37926960 | 31478 | 6:37928156 |
| 31484 | 6:37929786 | 31486 | 6:37930252 | 31487 | 6:37930293 | 31488 | 6:37930672 | 31490 | 6:37930868 |
| 31491 | 6:37930889 | 31492 | 6:37931089 | 31493 | 6:37931216 | 31494 | 6:37931298 | 31495 | 6:37931621 |
| 31496 | 6:37931688 | 31499 | 6:37932170 | 31501 | 6:37932241 | 31503 | 6:37932377 | 31504 | 6:37932412 |
| 31506 | 6:37932563 | 31507 | 6:37932682 | 31508 | 6:37932772 | 31509 | 6:37932778 | 31510 | 6:37932960 |
| 31512 | 6:37933352 | 31514 | 6:37934068 | 31518 | 6:37935108 | 31519 | 6:37935168 | 31520 | 6:37935527 |
| 31521 | 6:37935577 | 31522 | 6:37935748 | 31524 | 6:37936087 | 31525 | 6:37936692 | 31526 | 6:37938294 |
| 31527 | 6:37938374 | 31528 | 6:37938826 | 31531 | 6:37942290 | 31532 | 6:37942944 | 31533 | 6:37943635 |
| 31534 | 6:37943860 | 31535 | 6:37943970 | 31536 | 6:37944478 | 31537 | 6:37944482 | 31538 | 6:37944496 |
| 31539 | 6:37944573 | 31541 | 6:37944681 | 31542 | 6:37944704 | 31544 | 6:37945482 | 31545 | 6:37945820 |
| 31546 | 6:37945849 | 31558 | 6:37959360 | 31561 | 6:37961553 | 31563 | 6:37962887 | 31565 | 6:37963525 |
| 31568 | 6:37966382 | 31585 | 6:37974401 | 31586 | 6:37974407 | 31819 | 6:38395086 | 31822 | 6:38409517 |
| 31826 | 6:38491475 | 31831 | 6:38518347 | 31836 | 6:38540452 | 31837 | 6:38541525 | 31839 | 6:38542048 |
| 31842 | 6:38543400 | 31843 | 6:38543545 | 31844 | 6:38543990 | 31845 | 6:38545112 | 31846 | 6:38545113 |
| 31847 | 6:38546290 | 31848 | 6:38546646 | 31849 | 6:38548060 | 31850 | 6:38548198 | 31851 | 6:38548496 |
| 31852 | 6:38548525 | 31853 | 6:38548659 | 31854 | 6:38549044 | 31855 | 6:38549099 | 31856 | 6:38549206 |
| 31857 | 6:38549477 | 31859 | 6:38550162 | 31860 | 6:38550667 | 31861 | 6:38552085 | 31862 | 6:38552158 |
| 31863 | 6:38552167 | 31864 | 6:38552459 | 31865 | 6:38552550 | 31866 | 6:38552644 | 31867 | 6:38552821 |
| 31868 | 6:38553028 | 31869 | 6:38553181 | 31870 | 6:38553447 | 31871 | 6:38553456 | 31872 | 6:38553565 |
| 31873 | 6:38553842 | 31874 | 6:38553946 | 31875 | 6:38554100 | 31876 | 6:38554163 | 31877 | 6:38554439 |
| 31878 | 6:38554455 | 31879 | 6:38554714 | 31880 | 6:38555555 | 31881 | 6:38556220 | 31882 | 6:38556748 |
| 31883 | 6:38556956 | 31884 | 6:38556978 | 31885 | 6:38557241 | 31886 | 6:38557498 | 31887 | 6:38557633 |
| 31888 | 6:38557694 | 31889 | 6:38557830 | 31890 | 6:38557833 | 31891 | 6:38557888 | 31892 | 6:38557940 |
| 31894 | 6:38558946 | 31895 | 6:38562229 | 31897 | 6:38564166 | 31898 | 6:38565084 | 31902 | 6:38583433 |
| 31904 | 6:38584686 | 31908 | 6:38600235 | 31965 | 6:39250952 | 31969 | 6:39301737 | 31989 | 6:39663383 |
| 31992 | 6:39721697 | 31993 | 6:39727899 | 31994 | 6:39730068 | 31995 | 6:39733610 | 31996 | 6:39737879 |
| 32029 | 6:40129309 | 32034 | 6:40237863 | 32062 | 6:40696320 | 32086 | 6:40735514 | 32119 | 6:40952449 |
| 32207 | 6:42123221 | 32266 | 6:43009159 | 32267 | 6:43017852 | 32294 | 6:43462079 | 32329 | 6:43973189 |
| 32339 | 6:44149286 | 32547 | 6:46220665 | 32588 | 6:47190072 | 32619 | 6:47907662 | 32631 | 6:48281817 |
| 32657 | 6:49188655 | 32670 | 6:49492652 | 32675 | 6:49628351 | 32678 | 6:49692019 | 32757 | 6:51655726 |
| 32824 | 6:52798083 | 32827 | 6:52912323 | 32854 | 6:53474037 | 32911 | 6:55287740 | 32947 | 6:55553127 |
| 32949 | 6:55563964 | 32951 | 6:55573386 | 32968 | 6:56049754 | 32972 | 6:56230949 | 33026 | 6:57612544 |
| 33034 | 6:57940998 | 33036 | 6:57996603 | 33038 | 6:58046666 | 33086 | 6:59279953 | 33101 | 6:59756158 |
| 33144 | 6:60551717 | 33151 | 6:60729399 | 33174 | 6:61339528 | 33199 | 6:61996360 | 33215 | 6:62563366 |
| 33218 | 6:62627183 | 33317 | 6:65798913 | 33363 | 6:67312644 | 33364 | 6:67348632 | 33404 | 6:68600503 |
| 33421 | 6:69030371 | 33437 | 6:69395469 | 33438 | 6:69429025 | 33455 | 6:70069795 | 33480 | 6:70868126 |
| 33492 | 6:71178118 | 33506 | 6:71614584 | 33509 | 6:71658863 | 33575 | 6:73737226 | 33611 | 6:75157043 |
| 33635 | 6:75976163 | 33715 | 6:78625022 | 33873 | 6:83484610 | 33909 | 6:84465355 | 33927 | 6:84898498 |
| 34137 | 6:89248038 | 34177 | 6:90260850 | 34180 | 6:90360832 | 34292 | 6:92070503 | 34356 | 6:93561855 |
| 34416 | 6:94977849 | 34431 | 6:95495492 | 34452 | 6:96090013 | 34453 | 6:96097237 | 34476 | 6:96479912 |
| 34478 | 6:96544331 | 34486 | 6:96686083 | 34491 | 6:96916348 | 34531 | 6:97462721 | 34545 | 6:97701064 |
| 34600 | 6:98712630 | 34635 | 6:99753620 | 34663 | 6:100291469 | 34716 | 6:101484421 | 34717 | 6:101486616 |
| 34720 | 6:101524578 | 34735 | 6:101873336 | 34761 | 6:102268756 | 34817 | 6:103176226 | 34828 | 6:103236460 |
| 34912 | 6:105003956 | 35080 | 6:109427077 | 35101 | 6:109822389 | 35163 | 6:111118988 | 35165 | 6:111196630 |
| 35175 | 6:111382228 | 35229 | 6:112604266 | 35271 | 6:113397576 | 35275 | 6:113470683 | 35406 | 6:115996386 |
| 35448 | 6:116540874 | 35480 | 7:310937 | 35482 | 7:325799 | 35558 | 7:1927986 | 35561 | 7:1995591 |
| 35582 | 7:2406915 | 35845 | 7:6222553 | 35873 | 7:6554748 | 35887 | 7:6745523 | 35900 | 7:6956627 |
| 35928 | 7:7534130 | 35936 | 7:7674523 | 35947 | 7:8035405 | 35989 | 7:10310655 | 35990 | 7:10395846 |
| 36049 | 7:11976493 | 36068 | 7:12333943 | 36069 | 7:12335290 | 36140 | 7:13300617 | 36153 | 7:13562003 |
| 36173 | 7:13942658 | 36277 | 7:15601726 | 36310 | 7:16447387 | 36311 | 7:16487854 | 36362 | 7:17629628 |
| 36364 | 7:17639856 | 36392 | 7:18356451 | 36423 | 7:19107522 | 36440 | 7:19307460 | 36503 | 7:20153088 |
| 36525 | 7:20401996 | 36546 | 7:20912796 | 36597 | 7:22016441 | 36603 | 7:22057135 | 36620 | 7:22504243 |
| 36646 | 7:23221425 | 36663 | 7:23603215 | 36683 | 7:24060066 | 36685 | 7:24072015 | 36705 | 7:24634875 |
| 36768 | 7:26043336 | 36828 | 7:27103526 | 36858 | 7:27828159 | 36879 | 7:28407768 | 36890 | 7:28661620 |
| 36941 | 7:29715806 | 37014 | 7:31333882 | 37042 | 7:31763022 | 37047 | 7:31806849 | 37062 | 7:32255462 |
| 37091 | 7:33083761 | 37108 | 7:33452281 | 37175 | 7:35015402 | 37188 | 7:35370637 | 37211 | 7:35997017 |
| 37230 | 7:36507816 | 37294 | 7:37977197 | 37369 | 7:39696107 | 37371 | 7:39720514 | 37528 | 7:43355224 |
| 37544 | 7:43621545 | 37609 | 7:45530009 | 37635 | 7:46401887 | 37725 | 7:48628543 | 37738 | 7:48808046 |
| 37745 | 7:48945061 | 37752 | 7:49169638 | 37886 | 7:52686851 | 37912 | 7:53315105 | 37957 | 7:54541065 |
| 37960 | 7:54622430 | 37975 | 7:55088638 | 38007 | 7:55900961 | 38015 | 7:56078682 | 38033 | 7:56535413 |
| 38035 | 7:56546344 | 38057 | 7:57273769 | 38076 | 7:57910079 | 38077 | 7:57916334 | 38081 | 7:57993375 |
| 38083 | 7:58043785 | 38153 | 7:59374018 | 38159 | 7:59461244 | 38188 | 7:60116908 | 38196 | 7:60319670 |
| 38224 | 7:61001713 | 38269 | 7:61575267 | 38286 | 7:62059589 | 38299 | 7:62381127 | 38312 | 7:62665355 |
| 38313 | 7:62666818 | 38441 | 7:65409794 | 38449 | 7:65594792 | 38520 | 7:66847524 | 38592 | 7:68772935 |
| 38594 | 7:68885530 | 38657 | 7:70110273 | 38683 | 7:71032462 | 38718 | 7:71794897 | 38728 | 7:71996424 |
| 38742 | 7:72314529 | 38808 | 7:74314108 | 38814 | 7:74538065 | 38843 | 7:75119443 | 38849 | 7:75246858 |
| 38850 | 7:75278782 | 38865 | 7:75680840 | 38908 | 7:76699078 | 38924 | 7:77064173 | 38949 | 7:77643339 |
| 38981 | 7:78247892 | 38986 | 7:78358118 | 39067 | 7:79874457 | 39117 | 7:80683144 | 39127 | 7:80972557 |
| 39147 | 7:81218151 | 39197 | 7:82527838 | 39256 | 7:83861078 | 39276 | 7:84283959 | 39317 | 7:85606256 |
| 39369 | 7:87039982 | 39386 | 7:87585340 | 39438 | 7:89078282 | 39458 | 7:89409840 | 39473 | 7:89883975 |
| 39495 | 7:90326242 | 39589 | 7:91305083 | 39633 | 7:92190820 | 39704 | 7:94482521 | 39741 | 7:95245462 |
| 39764 | 7:95873877 | 39776 | 7:96101255 | 39780 | 7:96126223 | 39967 | 7:96962124 | 39977 | 7:97249811 |
| 39984 | 7:97508045 | 39987 | 7:97537955 | 40007 | 7:98034786 | 40009 | 7:98127942 | 40167 | 7:102360026 |
| 40187 | 7:102838629 | 40190 | 7:102879057 | 40252 | 7:104220122 | 40270 | 7:104812484 | 40345 | 7:106558358 |
| 40381 | 7:107255212 | 40401 | 7:108080872 | 40406 | 7:108274048 | 40425 | 7:108662049 | 40435 | 7:108882531 |
| 40540 | 8:1185446 | 40561 | 8:1731044 | 40562 | 8:1735452 | 40568 | 8:1797826 | 40584 | 8:2112938 |
| 40597 | 8:2381761 | 40609 | 8:2664904 | 40720 | 8:5502050 | 40787 | 8:7227540 | 40820 | 8:7866886 |
| 40852 | 8:8701133 | 40988 | 8:11839675 | 41027 | 8:12939122 | 41083 | 8:14812117 | 41084 | 8:14836734 |
| 41129 | 8:15805194 | 41130 | 8:15826955 | 41140 | 8:16188640 | 41161 | 8:16768290 | 41295 | 8:20088784 |
| 41367 | 8:22206603 | 41372 | 8:22296675 | 41379 | 8:22531070 | 41384 | 8:22832882 | 41391 | 8:23108057 |
| 41574 | 8:25836147 | 41585 | 8:26072917 | 41683 | 8:28986201 | 41684 | 8:29004667 | 41685 | 8:29005955 |
| 41710 | 8:29655591 | 41727 | 8:29969758 | 41806 | 8:32023600 | 41820 | 8:32402575 | 41826 | 8:32658867 |
| 41872 | 8:34205302 | 41887 | 8:34586231 | 41924 | 8:35462325 | 41970 | 8:36379322 | 41971 | 8:36399226 |
| 42048 | 8:38716745 | 42085 | 8:39915480 | 42086 | 8:39942972 | 42119 | 8:40483328 | 42137 | 8:40876799 |
| 42195 | 8:42367753 | 42266 | 8:44367476 | 42313 | 8:45640500 | 42355 | 8:46379679 | 42373 | 8:46931988 |
| 42389 | 8:47348455 | 42415 | 8:47938185 | 42433 | 8:48578484 | 42492 | 8:50146172 | 42546 | 8:51588079 |
| 42631 | 8:53806641 | 42639 | 8:54114529 | 42646 | 8:54256907 | 42654 | 8:54525130 | 42656 | 8:54533690 |
| 42657 | 8:54534313 | 42658 | 8:54536088 | 42659 | 8:54544973 | 42660 | 8:54547153 | 42854 | 8:59769666 |
| 42892 | 8:60472436 | 42903 | 8:60785213 | 43085 | 8:65667278 | 43089 | 8:65893239 | 43110 | 8:66320758 |
| 43186 | 8:67933084 | 43215 | 8:68749952 | 43230 | 8:69106626 | 43232 | 8:69110799 | 43244 | 8:69435453 |
| 43345 | 8:72058951 | 43397 | 8:73337772 | 43434 | 8:74223661 | 43566 | 8:77624055 | 43579 | 8:78130709 |
| 43593 | 8:78494713 | 43655 | 8:80608965 | 43668 | 8:80925878 | 43679 | 8:81135408 | 43745 | 8:82415599 |
| 43764 | 8:82948559 | 43847 | 8:85052405 | 43927 | 8:87126956 | 43963 | 8:87960883 | 43972 | 8:88206973 |
| 43973 | 8:88207717 | 44068 | 8:90411690 | 44089 | 8:90899216 | 44097 | 8:91046537 | 44301 | 8:96913052 |
| 44325 | 8:97476895 | 44365 | 8:98665857 | 44390 | 8:99176725 | 44398 | 8:99311311 | 44414 | 8:99547619 |
| 44418 | 8:99577548 | 44438 | 8:100015023 | 44490 | 8:101376581 | 44635 | 8:103813682 | 44653 | 8:104231786 |
| 44746 | 8:106532357 | 44749 | 8:106594158 | 44792 | 8:107582608 | 45042 | 9:2688546 | 45058 | 9:3203477 |
| 45130 | 9:5470319 | 45151 | 9:6116416 | 45173 | 9:6877480 | 45177 | 9:7031515 | 45229 | 9:8388451 |
| 45283 | 9:9884501 | 45296 | 9:10301651 | 45298 | 9:10310168 | 45417 | 9:11927500 | 45439 | 9:12374207 |
| 45514 | 9:13742514 | 45529 | 9:14218593 | 45532 | 9:14239370 | 45566 | 9:15019469 | 45593 | 9:15465522 |
| 45684 | 9:17366049 | 45686 | 9:17428879 | 45709 | 9:18143920 | 45711 | 9:18153000 | 45712 | 9:18160118 |
| 45757 | 9:19618032 | 45977 | 9:25228881 | 45987 | 9:25512136 | 46000 | 9:25838484 | 46031 | 9:26618713 |
| 46034 | 9:26691579 | 46063 | 9:27473901 | 46065 | 9:27477523 | 46066 | 9:27485623 | 46095 | 9:28146296 |
| 46123 | 9:28679350 | 46135 | 9:28927068 | 46242 | 9:30976439 | 46245 | 9:31003562 | 46276 | 9:31748693 |
| 46330 | 9:33062497 | 46343 | 9:33376755 | 46364 | 9:33886935 | 46372 | 9:34155670 | 46516 | 9:37778578 |
| 46545 | 9:38309457 | 46546 | 9:38314365 | 46561 | 9:38550987 | 46644 | 9:40310425 | 46767 | 9:43367400 |
| 46771 | 9:43430747 | 46802 | 9:44301570 | 46864 | 9:46172575 | 46865 | 9:46201814 | 46916 | 9:47818319 |
| 46943 | 9:48865487 | 46946 | 9:48902577 | 46963 | 9:49476706 | 46974 | 9:49653803 | 47048 | 9:51753467 |
| 47067 | 9:52274853 | 47097 | 9:52875308 | 47148 | 9:54342081 | 47149 | 9:54361703 | 47168 | 9:54807613 |
| 47325 | 9:59403628 | 4734 | 9:59637965 | 47341 | 9:59638253 | 47342 | 9:59641031 | 47343 | 9:59643481 |
| 47344 | 9:59645062 | 47345 | 9:59645804 | 47346 | 9:59647105 | 47347 | 9:59650376 | 47349 | 9:59653721 |
| 47385 | 9:60709648 | 47395 | 9:60924554 | 47396 | 9:60926410 | 47425 | 9:61795735 | 47611 | 9:65961632 |
| 47667 | 9:67443259 | 47669 | 9:67511922 | 47679 | 9:67708264 | 47688 | 9:68029230 | 47727 | 9:69025344 |
| 47770 | 9:70119558 | 47782 | 9:70360272 | 47843 | 9:71633299 | 47985 | 9:75491653 | 48031 | 9:76631634 |
| 48186 | 9:79336612 | 48219 | 9:80142294 | 48257 | 9:80938431 | 48267 | 9:81108364 | 48274 | 9:81217242 |
| 48305 | 9:81845883 | 48312 | 9:82017648 | 48340 | 9:82626166 | 48342 | 9:82647502 | 48343 | 9:82651491 |
| 48345 | 9:82668654 | 48348 | 9:82682681 | 48354 | 9:82701768 | 48361 | 9:82842810 | 48394 | 9:83693006 |
| 48397 | 9:83826419 | 48468 | 9:85456175 | 48490 | 9:85784712 | 48550 | 9:87315819 | 48553 | 9:87327271 |
| 48558 | 9:87361936 | 48559 | 9:87373306 | 48590 | 9:87819557 | 48591 | 9:87822671 | 48627 | 9:88634584 |
| 48631 | 9:88656403 | 48632 | 9:88680123 | 48707 | 9:89908123 | 48708 | 9:89918140 | 48710 | 9:89921487 |
| 48713 | 9:90012380 | 48748 | 9:90826907 | 48780 | 9:91531504 | 48810 | 9:92215180 | 48815 | 9:92295269 |
| 48856 | 9:93221725 | 48880 | 9:93749307 | 48891 | 9:93889254 | 48911 | 9:94252780 | 48914 | 9:94269583 |
| 48923 | 9:94459307 | 4892 | 9:94493145 | 48986 | 9:95568256 | 49048 | 9:96604431 | 49052 | 9:96651231 |
| 49053 | 9:96652474 | 49054 | 9:96655491 | 49055 | 9:96658405 | 49056 | 9:96659811 | 49059 | 9:96727179 |
| 49060 | 9:96730432 | 49065 | 9:96818068 | 49100 | 9:97292214 | 49186 | 9:99056298 | 49199 | 9:99304134 |
| 49204 | 9:99370466 | 49267 | 9:100494397 | 49269 | 9:100511637 | 49277 | 9:100658454 | 49280 | 9:100721863 |
| 49342 | 9:101817009 | 49343 | 9:101818874 | 49344 | 9:101822513 | 49346 | 9:101827562 | 49349 | 9:101865213 |
| 49442 | 9:103263715 | 49469 | 9:103800979 | 49529 | 10:872754 | 49531 | 10:882204 | 49542 | 10:1178970 |
| 49569 | 10:1795897 | 49614 | 10:2626851 | 49654 | 10:3355041 | 49730 | 10:4924755 | 49746 | 10:5330263 |
| 49804 | 10:6194603 | 49811 | 10:6389416 | 49855 | 10:7506260 | 49856 | 10:7508517 | 49896 | 10:8296974 |
| 49909 | 10:8505165 | 49939 | 10:9303914 | 49984 | 10:10211405 | 49995 | 10:10425877 | 50019 | 10:10791977 |
| 50022 | 10:10887987 | 50067 | 10:11918946 | 50097 | 10:12461852 | 50162 | 10:13597734 | 50172 | 10:13748622 |
| 50235 | 10:15125993 | 50326 | 10:16719617 | 50347 | 10:17070966 | 50355 | 10:17235899 | 50385 | 10:17893631 |
| 50510 | 10:20312528 | 50512 | 10:20320283 | 50597 | 10:21831782 | 50627 | 10:22355891 | 50730 | 10:26724399 |
| 50740 | 10:26816725 | 50748 | 10:26936267 | 50763 | 10:27119455 | 50820 | 10:28726853 | 50827 | 10:28818762 |
| 50840 | 10:28923770 | 50844 | 10:28933854 | 50884 | 10:29514735 | 50885 | 10:29522253 | 50886 | 10:29533360 |
| 50906 | 10:30075494 | 50909 | 10:30126897 | 50911 | 10:30167171 | 50955 | 10:31703939 | 50968 | 10:32063661 |
| 50983 | 10:32536622 | 51010 | 10:33047373 | 51015 | 10:33221086 | 51073 | 10:34533777 | 51093 | 10:34950052 |
| 51125 | 10:35727830 | 51189 | 10:37345559 | 51190 | 10:37377718 | 51191 | 10:37388003 | 51202 | 10:37683947 |
| 51210 | 10:37775786 | 51223 | 10:38166114 | 51227 | 10:38269447 | 51243 | 10:38653361 | 51265 | 10:39263333 |
| 51295 | 10:40078678 | 51332 | 10:41115727 | 51348 | 10:41693430 | 51354 | 10:41758465 | 51445 | 10:44433737 |
| 51454 | 10:44645299 | 51494 | 10:45580212 | 51611 | 10:47920359 | 51626 | 10:48320530 | 51643 | 10:48547551 |
| 51688 | 10:49516649 | 51721 | 10:50223805 | 51725 | 10:50334170 | 51759 | 10:51169084 | 51760 | 10:51181102 |
| 51761 | 10:51186753 | 51773 | 10:51492328 | 51784 | 10:51754878 | 51809 | 10:52286224 | 51840 | 10:52879725 |
| 51889 | 10:54463903 | 51952 | 10:56290718 | 51963 | 10:56443542 | 52006 | 10:57713193 | 52037 | 10:58360318 |
| 52049 | 10:58658013 | 52120 | 10:61117665 | 52181 | 10:62385028 | 52245 | 10:64094738 | 52272 | 10:64614710 |
| 52276 | 10:64656067 | 52345 | 10:66304541 | 52390 | 10:67276588 | 52391 | 10:67310241 | 52399 | 10:67673108 |
| 52467 | 10:68971489 | 52480 | 10:69334027 | 52486 | 10:69501262 | 52499 | 10:69823517 | 52501 | 10:69836682 |
| 52504 | 10:69941547 | 52513 | 10:70197389 | 52537 | 10:71023784 | 52552 | 10:71449748 | 52570 | 10:72023102 |
| 52598 | 10:73101962 | 52623 | 10:73660537 | 52653 | 10:74459889 | 52665 | 10:75024558 | 52672 | 10:75197354 |
| 52688 | 10:75607741 | 52743 | 10:76813636 | 52823 | 10:78537516 | 52826 | 10:78594348 | 52840 | 10:79035728 |
| 52900 | 10:80684424 | 52913 | 10:81068664 | 52970 | 10:81760396 | 53006 | 10:82276845 | 53023 | 10:82877025 |
| 53054 | 10:83522156 | 53055 | 10:83550757 | 53062 | 10:83712797 | 53068 | 10:83857093 | 53071 | 10:83936676 |
| 53099 | 10:84417728 | 53239 | 10:87507691 | 53287 | 10:88767077 | 53305 | 10:89008507 | 53312 | 10:89144342 |
| 53340 | 10:89729222 | 53366 | 10:90207696 | 53388 | 10:90615371 | 53487 | 10:92661034 | 53497 | 10:92892557 |
| 53545 | 10:94160502 | 53555 | 10:94348103 | 53604 | 10:95590054 | 53623 | 10:95920911 | 53627 | 10:96023548 |
| 53669 | 10:97104354 | 53675 | 10:97169425 | 53745 | 10:98522222 | 53797 | 10:99634106 | 53869 | 10:101120062 |
| 53875 | 10:101177760 | 53882 | 10:101281677 | 53887 | 10:101426015 | 53913 | 10:101819543 | 53944 | 10:102268731 |
| 54003 | 11:441012 | 54045 | 11:1204347 | 54069 | 11:1549638 | 54111 | 11:1989902 | 54174 | 11:2689033 |
| 54209 | 11:3235907 | 54259 | 11:4054643 | 54292 | 11:4984752 | 54300 | 11:5106453 | 54327 | 11:5677169 |
| 54409 | 11:7670104 | 54425 | 11:8021628 | 54476 | 11:9219377 | 54572 | 11:11342389 | 54630 | 11:12715868 |
| 54638 | 11:12870807 | 54674 | 11:13642677 | 54677 | 11:13704625 | 54736 | 11:15343003 | 54745 | 11:15626371 |
| 54747 | 11:15658858 | 54756 | 11:15853957 | 54797 | 11:16775998 | 54856 | 11:18554101 | 54872 | 11:18844419 |
| 54875 | 11:18948217 | 54883 | 11:19173839 | 54923 | 11:20562842 | 54924 | 11:20568845 | 54981 | 11:21832414 |
| 55037 | 11:23157077 | 55048 | 11:23494343 | 55084 | 11:24320520 | 55092 | 11:24369984 | 55093 | 11:24377983 |
| 55094 | 11:24393545 | 55096 | 11:24408850 | 55119 | 11:24944857 | 55151 | 11:25930515 | 55161 | 11:26154577 |
| 55174 | 11:26362493 | 55214 | 11:27414983 | 55238 | 11:27959917 | 55283 | 11:28844947 | 55302 | 11:29230653 |
| 55315 | 11:29502309 | 55352 | 11:30379793 | 55380 | 11:30989937 | 55381 | 11:31003265 | 55382 | 11:31020736 |
| 55383 | 11:31023833 | 55425 | 11:32229026 | 55458 | 11:33116089 | 55479 | 11:33643610 | 55496 | 11:34099362 |
| 55532 | 11:35548594 | 55538 | 11:35660110 | 55568 | 11:36429662 | 55588 | 11:37003872 | 55608 | 11:37326478 |
| 55615 | 11:37508364 | 55620 | 11:37570757 | 55624 | 11:37639983 | 55768 | 11:41573665 | 55824 | 11:42850632 |
| 55835 | 11:43167995 | 55873 | 11:44279092 | 55880 | 11:44503647 | 55915 | 11:45256103 | 55925 | 11:45835142 |
| 55933 | 11:45970733 | 55938 | 11:46039317 | 55941 | 11:46045608 | 55942 | 11:46058932 | 55972 | 11:46675342 |
| 56017 | 11:47326731 | 56043 | 11:47992234 | 56067 | 11:48522658 | 56126 | 11:49051072 | 56190 | 11:50710309 |
| 56222 | 11:51848453 | 56258 | 11:53253320 | 56273 | 11:53826835 | 56332 | 11:55052140 | 56335 | 11:55137862 |
| 56337 | 11:55149593 | 56397 | 11:56859473 | 56653 | 11:63292715 | 56682 | 11:63832921 | 56683 | 11:63837249 |
| 56714 | 11:64511310 | 56734 | 11:65119714 | 56739 | 11:65250246 | 56752 | 11:65585594 | 56764 | 11:65826676 |
| 56767 | 11:66038610 | 56784 | 11:66319597 | 56812 | 11:67082495 | 56896 | 11:68461614 | 56913 | 11:68937779 |
| 56917 | 11:69060122 | 56924 | 11:69109200 | 56925 | 11:69118745 | 56928 | 11:69142947 | 56933 | 11:69181613 |
| 56943 | 11:69335989 | 57008 | 11:70675473 | 57082 | 11:71401956 | 57156 | 11:73126654 | 57200 | 11:74123227 |
| 57271 | 11:75902686 | 57350 | 11:77659975 | 57451 | 11:79225059 | 57506 | 11:80496876 | 57543 | 11:81586331 |
| 57554 | 11:81811191 | 57559 | 11:81888932 | 57573 | 11:82234600 | 57724 | 11:85632013 | 57806 | 11:87517972 |
| 57868 | 11:88067171 | 57900 | 11:88940240 | 57915 | 11:89270730 | 57969 | 11:90490496 | 57973 | 11:90559289 |
| 57995 | 11:91106611 | 58020 | 11:91612293 | 58045 | 11:92156116 | 58120 | 11:93813789 | 58133 | 11:94097451 |
| 58153 | 11:94563151 | 58180 | 11:95385268 | 58195 | 11:95727670 | 58219 | 11:96378020 | 58238 | 11:97095593 |
| 58246 | 11:97197864 | 58308 | 11:98538984 | 58364 | 11:99473375 | 58417 | 11:100532361 | 58536 | 11:102829142 |
| 58591 | 11:103534827 | 58607 | 11:104097856 | 58720 | 11:106485352 | 58877 | 12:3502498 | 58943 | 12:5186588 |
| 58945 | 12:5325638 | 58966 | 12:6004879 | 58987 | 12:6773018 | 59008 | 12:7507940 | 59043 | 12:8830007 |
| 59057 | 12:9272154 | 59061 | 12:9329563 | 59069 | 12:9522013 | 59119 | 12:10577179 | 59120 | 12:10585260 |
| 59155 | 12:11315928 | 59176 | 12:11720838 | 59179 | 12:11790945 | 59182 | 12:11827023 | 59184 | 12:11857024 |
| 59208 | 12:12366690 | 59238 | 12:13384011 | 59260 | 12:13823314 | 59276 | 12:14077064 | 59282 | 12:14150866 |
| 59301 | 12:14486563 | 59316 | 12:14918565 | 59397 | 12:16476525 | 59417 | 12:16974977 | 59428 | 12:17253883 |
| 59434 | 12:17359694 | 59439 | 12:17465924 | 59454 | 12:17933719 | 59471 | 12:18284739 | 59549 | 12:20109323 |
| 59698 | 12:23940231 | 59702 | 12:24068026 | 59727 | 12:24577728 | 59786 | 12:25839029 | 59799 | 12:26457768 |
| 59840 | 12:27377754 | 59846 | 12:27488955 | 59870 | 12:28122480 | 59956 | 12:30649835 | 59989 | 12:31264310 |
| 60008 | 12:31780482 | 60117 | 12:34750935 | 60139 | 12:35299411 | 60141 | 12:35311925 | 60142 | 12:35312992 |
| 60143 | 12:35314580 | 60144 | 12:35321842 | 60145 | 12:35326856 | 60147 | 12:35334692 | 60148 | 12:35339381 |
| 60149 | 12:35342141 | 60151 | 12:35355118 | 60152 | 12:35355785 | 60153 | 12:35363362 | 60154 | 12:35424126 |
| 60156 | 12:35487159 | 60202 | 12:37059151 | 60206 | 12:37121439 | 60347 | 12:42536086 | 60352 | 12:42838412 |
| 60389 | 12:44505965 | 60458 | 12:46806766 | 60555 | 12:49288668 | 60578 | 12:49872127 | 60583 | 12:49903248 |
| 60596 | 12:50116234 | 60604 | 12:50253408 | 60730 | 12:53098596 | 60840 | 12:55588645 | 60862 | 12:56409050 |
| 60875 | 12:56736797 | 60935 | 12:58879036 | 61043 | 12:62502730 | 61081 | 12:63602083 | 61084 | 12:63713299 |
| 61163 | 12:65908229 | 61179 | 12:66475638 | 61325 | 12:69535302 | 61424 | 12:73231474 | 61483 | 12:74215241 |
| 61488 | 12:74344277 | 61565 | 12:76057266 | 61574 | 12:76294965 | 61575 | 12:76298488 | 61597 | 12:76881665 |
| 61771 | 12:80709446 | 61777 | 12:80884100 | 61814 | 12:81734916 | 61831 | 12:82087879 | 61844 | 12:82441644 |
| 61856 | 12:82591128 | 61912 | 12:83854133 | 61961 | 12:84933491 | 61993 | 12:85357065 | 62129 | 13:1206621 |
| 62198 | 13:2453852 | 62215 | 13:2711565 | 62223 | 13:2835285 | 62289 | 13:4735864 | 62347 | 13:5936143 |
| 62362 | 13:6285905 | 62525 | 13:10419094 | 62677 | 13:14935831 | 62696 | 13:15681048 | 62771 | 13:17258775 |
| 62815 | 13:17832527 | 62916 | 13:19454627 | 62947 | 13:19907032 | 62968 | 13:20390279 | 62969 | 13:20405413 |
| 62970 | 13:20411494 | 62971 | 13:20423875 | 62972 | 13:20430234 | 62988 | 13:20701775 | 63002 | 13:21060977 |
| 63055 | 13:22024146 | 63112 | 13:23989482 | 63141 | 13:24644133 | 63143 | 13:24667549 | 63150 | 13:24857890 |
| 63174 | 13:25338856 | 63181 | 13:25455632 | 63193 | 13:25724492 | 63199 | 13:25901617 | 63240 | 13:27082022 |
| 63282 | 13:27909495 | 63285 | 13:28007570 | 63295 | 13:28335861 | 63305 | 13:28545136 | 63341 | 13:29355083 |
| 63359 | 13:29815935 | 63424 | 13:31076841 | 63427 | 13:31114025 | 63436 | 13:31276115 | 63470 | 13:31684931 |
| 63484 | 13:32188070 | 63485 | 13:32192701 | 63486 | 13:32195543 | 63488 | 13:32202239 | 63490 | 13:32214896 |
| 63507 | 13:32509667 | 63513 | 13:32595507 | 63515 | 13:32606184 | 63519 | 13:32649822 | 63520 | 13:32651100 |
| 63644 | 13:35586171 | 63673 | 13:36380278 | 63730 | 13:37696035 | 63799 | 13:39119033 | 63842 | 13:39974325 |
| 63843 | 13:40001169 | 63848 | 13:40028952 | 63884 | 13:40680902 | 63923 | 13:41808005 | 63934 | 13:41965173 |
| 63957 | 13:42527459 | 64015 | 13:43212135 | 64028 | 13:43604201 | 64041 | 13:43854540 | 64042 | 13:43855779 |
| 64043 | 13:43868655 | 64044 | 13:43869987 | 64045 | 13:43870636 | 64046 | 13:43872533 | 64047 | 13:43873040 |
| 64048 | 13:43873873 | 64049 | 13:43876772 | 64050 | 13:43878363 | 64051 | 13:43879388 | 64052 | 13:43880125 |
| 64053 | 13:43884214 | 64258 | 13:47680398 | 64280 | 13:48210593 | 64321 | 13:49204706 | 64383 | 13:51177851 |
| 64397 | 13:51341948 | 64439 | 13:52703815 | 64510 | 13:53977365 | 64538 | 13:54845724 | 64978 | 13:64716430 |
| 64982 | 13:64796939 | 64998 | 13:65331493 | 65047 | 13:66193660 | 65061 | 13:66407606 | 65093 | 13:67181101 |
| 65096 | 13:67321578 | 65215 | 13:70526426 | 65221 | 13:70575324 | 65237 | 13:70871738 | 65271 | 13:71637090 |
| 65328 | 13:72957542 | 65357 | 13:73471150 | 65381 | 13:73812183 | 65456 | 13:75531070 | 65529 | 13:76798256 |
| 65547 | 13:77244637 | 65550 | 13:77279091 | 65578 | 13:77845959 | 65598 | 13:78300577 | 65660 | 13:79330804 |
| 65677 | 13:79542295 | 65819 | 13:82171173 | 65917 | 14:1204169 | 65929 | 14:1645654 | 66049 | 14:3651551 |
| 66072 | 14:4085451 | 66078 | 14:4200331 | 66116 | 14:4961976 | 66160 | 14:5700495 | 66209 | 14:6316411 |
| 66268 | 14:7449642 | 66271 | 14:7527096 | 66317 | 14:8213069 | 66475 | 14:10912061 | 66523 | 14:11744884 |
| 66538 | 14:12080094 | 66544 | 14:12195646 | 66565 | 14:12541653 | 66566 | 14:12551654 | 66568 | 14:12570995 |
| 66627 | 14:14043986 | 66718 | 14:16010978 | 66719 | 14:16022809 | 66722 | 14:16058309 | 66732 | 14:16280608 |
| 66746 | 14:16568761 | 66820 | 14:18106996 | 66821 | 14:18115961 | 66855 | 14:19074429 | 66858 | 14:19172311 |
| 66877 | 14:19623114 | 66903 | 14:20293557 | 66912 | 14:20629249 | 66916 | 14:20662609 | 66917 | 14:20673221 |
| 66925 | 14:20792640 | 67108 | 14:22916046 | 67140 | 14:22986080 | 67146 | 14:22999212 | 67302 | 14:23338890 |
| 67351 | 14:23565143 | 67354 | 14:23600267 | 67355 | 14:23607944 | 67357 | 14:23630896 | 67359 | 14:23652804 |
| 67369 | 14:23826318 | 67371 | 14:23829066 | 67374 | 14:23831133 | 67501 | 14:24941221 | 67613 | 14:26283998 |
| 67660 | 14:27607287 | 67681 | 14:28330433 | 67709 | 14:29096850 | 67747 | 14:29873926 | 67772 | 14:30389540 |
| 67822 | 14:31570898 | 67845 | 14:32267064 | 67901 | 14:33278402 | 67955 | 14:34370911 | 68032 | 14:36313786 |
| 68053 | 14:36889990 | 68099 | 14:38260754 | 68128 | 14:39143238 | 68172 | 14:40259820 | 68190 | 14:40976286 |
| 68197 | 14:41229576 | 68198 | 14:41237524 | 68261 | 14:42746359 | 68276 | 14:43241727 | 68313 | 14:44566510 |
| 68321 | 14:44666155 | 68354 | 14:45237526 | 68360 | 14:45367206 | 68364 | 14:45505076 | 68368 | 14:45610432 |
| 68441 | 14:46995812 | 68448 | 14:47163738 | 68460 | 14:47428677 | 68478 | 14:48150783 | 68509 | 14:48767270 |
| 68536 | 14:49487643 | 68580 | 14:50970280 | 68581 | 14:50986924 | 68606 | 14:51659332 | 68620 | 14:51985037 |
| 68654 | 14:52815945 | 68668 | 14:53442111 | 68687 | 14:54022606 | 68730 | 14:54568169 | 68772 | 14:55536222 |
| 68826 | 14:57247647 | 68861 | 14:57991139 | 68870 | 14:58182404 | 68871 | 14:58201975 | 68907 | 14:58964090 |
| 68974 | 14:60591022 | 68975 | 14:60610629 | 68988 | 14:60975742 | 69085 | 14:62782594 | 69115 | 14:63450732 |
| 69130 | 14:63928126 | 69158 | 14:64538337 | 69184 | 14:65420223 | 69193 | 14:65688670 | 69200 | 14:65782382 |
| 69231 | 14:66405235 | 69239 | 14:66655367 | 69251 | 14:66916531 | 69316 | 14:68245567 | 69466 | 14:72276625 |
| 69482 | 14:72695548 | 69536 | 14:73988875 | 69639 | 14:76386785 | 69745 | 14:78754068 | 69764 | 14:79462729 |
| 69867 | 14:81514348 | 69890 | 14:81754335 | 69891 | 14:81755109 | 69932 | 15:549186 | 69952 | 15:1223740 |
| 69971 | 15:1763083 | 70074 | 15:4197179 | 70097 | 15:4603953 | 70120 | 15:5062052 | 70314 | 15:6267325 |
| 70382 | 15:7840165 | 70418 | 15:8668231 | 70426 | 15:8980586 | 70486 | 15:10462503 | 70525 | 15:11922022 |
| 70590 | 15:14196834 | 70637 | 15:15315326 | 70696 | 15:16104588 | 70710 | 15:16454917 | 70736 | 15:17101878 |
| 70782 | 15:18196851 | 70794 | 15:18357447 | 70804 | 15:18736931 | 70814 | 15:19007825 | 70838 | 15:19653453 |
| 70946 | 15:22147187 | 70997 | 15:23248430 | 71016 | 15:23621459 | 71018 | 15:23665519 | 71079 | 15:24372603 |
| 71091 | 15:24613968 | 71107 | 15:24837501 | 71123 | 15:25257089 | 71128 | 15:25308638 | 71168 | 15:26173379 |
| 71188 | 15:26573138 | 71308 | 15:28904879 | 71323 | 15:29119476 | 71357 | 15:30139511 | 71368 | 15:30396261 |
| 71408 | 15:31365259 | 71434 | 15:31780017 | 71443 | 15:32064162 | 71478 | 15:32883165 | 71517 | 15:33758501 |
| 71518 | 15:33767591 | 71580 | 15:34828367 | 71614 | 15:35688175 | 71626 | 15:35959116 | 71745 | 15:38587541 |
| 71763 | 15:38952840 | 71840 | 15:40889581 | 71862 | 15:41323112 | 71923 | 15:42472180 | 71943 | 15:42904887 |
| 72011 | 15:43795583 | 72016 | 15:43848104 | 72053 | 15:44559517 | 72177 | 15:46955071 | 72194 | 15:47343068 |
| 72237 | 15:48145511 | 72241 | 15:48207972 | 72244 | 15:48254047 | 72281 | 15:49497449 | 72308 | 15:50463550 |
| 72408 | 15:52214824 | 72429 | 15:52810027 | 72524 | 15:54359875 | 72578 | 15:55624075 | 72586 | 15:55875613 |
| 72693 | 15:57504930 | 72717 | 15:57932533 | 72747 | 15:58462557 | 72792 | 15:59699546 | 72793 | 15:59714205 |
| 72835 | 15:60939376 | 72865 | 15:61561772 | 72880 | 15:61802396 | 72881 | 15:61805665 | 72882 | 15:61806975 |
| 72885 | 15:61812433 | 72890 | 15:61960293 | 72891 | 15:61964427 | 72930 | 15:62856332 | 73076 | 15:65465351 |
| 73108 | 15:66183419 | 73192 | 15:68248786 | 73261 | 15:70440283 | 73286 | 15:71178088 | 73301 | 15:71472435 |
| 73332 | 15:72309114 | 73377 | 15:73291708 | 73393 | 15:73582243 | 73416 | 15:74163082 | 73451 | 15:74779756 |
| 73466 | 15:75089743 | 73510 | 15:76195722 | 73541 | 15:76835072 | 73630 | 15:78278239 | 73698 | 15:80309655 |
| 73754 | 15:81295713 | 73762 | 15:81438153 | 73813 | 15:82751747 | 73823 | 15:82918904 | 73880 | 15:83865721 |
| 73959 | 16:1501024 | 74086 | 16:4079477 | 74089 | 16:4114086 | 74111 | 16:4443184 | 74212 | 16:7478039 |
| 74284 | 16:10121160 | 74304 | 16:10857824 | 74327 | 16:11521662 | 74329 | 16:11621503 | 74349 | 16:12056975 |
| 74446 | 16:14409239 | 74451 | 16:14518175 | 74462 | 16:14841844 | 74479 | 16:15208552 | 74481 | 16:15279430 |
| 74498 | 16:15816016 | 74506 | 16:16013694 | 74511 | 16:16188270 | 74513 | 16:16194660 | 74578 | 16:18520385 |
| 74584 | 16:18637360 | 74591 | 16:18804747 | 74652 | 16:20427119 | 74853 | 16:25049887 | 74887 | 16:25231259 |
| 74902 | 16:25476855 | 74955 | 16:26802374 | 74956 | 16:26859375 | 74969 | 16:27035419 | 75000 | 16:27937410 |
| 75006 | 16:28023322 | 75055 | 16:29594638 | 75144 | 16:31915038 | 75148 | 16:31970232 | 75161 | 16:32424717 |
| 75174 | 16:32836939 | 75253 | 16:34714234 | 75284 | 16:35365207 | 75318 | 16:36337580 | 75342 | 16:36921696 |
| 75354 | 16:37184848 | 75457 | 16:39917217 | 75492 | 16:41063333 | 75508 | 16:41613390 | 75547 | 16:42048065 |
| 75564 | 16:42830906 | 75611 | 16:44815088 | 75636 | 16:45912914 | 75654 | 16:46353096 | 75728 | 16:48055006 |
| 75756 | 16:48627494 | 75757 | 16:48635114 | 75758 | 16:48637200 | 75759 | 16:48639734 | 75760 | 16:48645579 |
| 75761 | 16:48649527 | 75825 | 16:50275447 | 75834 | 16:50665850 | 75880 | 16:51861172 | 75887 | 16:52014418 |
| 75915 | 16:52578534 | 75975 | 16:54055090 | 75981 | 16:54147078 | 76006 | 16:54922205 | 76068 | 16:56374651 |
| 76087 | 16:56788303 | 76136 | 16:58112073 | 76149 | 16:58378141 | 76153 | 16:58406289 | 76198 | 16:59309602 |
| 76229 | 16:60126861 | 76246 | 16:60365696 | 76276 | 16:61171484 | 76282 | 16:61360408 | 76298 | 16:61585266 |
| 76320 | 16:62102938 | 76325 | 16:62226456 | 76366 | 16:63181951 | 76484 | 16:65323416 | 76487 | 16:65420109 |
| 76600 | 16:67626229 | 76631 | 16:68532430 | 76672 | 16:69359404 | 76687 | 16:69663665 | 76741 | 16:70858584 |
| 76747 | 16:70938743 | 76846 | 16:73114325 | 76890 | 16:73720962 | 76916 | 16:74372695 | 76921 | 16:74515053 |
| 77113 | 16:78293826 | 77116 | 16:78337900 | 77145 | 16:78921036 | 77151 | 16:78991296 | 77156 | 16:79028746 |
| 77158 | 16:79043302 | 77226 | 16:79983101 | 77237 | 16:80656680 | 77361 | 17:3014326 | 77369 | 17:3204260 |
| 77405 | 17:3947723 | 77459 | 17:4815417 | 77461 | 17:4875306 | 77475 | 17:5216472 | 77564 | 17:7040082 |
| 77625 | 17:8272941 | 77711 | 17:10199997 | 77712 | 17:10213106 | 78005 | 17:17163300 | 78016 | 17:17334505 |
| 78018 | 17:17357791 | 78027 | 17:17451464 | 78083 | 17:18818415 | 78126 | 17:19479964 | 78154 | 17:20220411 |
| 78164 | 17:20579995 | 78176 | 17:21008303 | 78218 | 17:22333330 | 78297 | 17:25079038 | 78310 | 17:25505355 |
| 78350 | 17:26610845 | 78351 | 17:26613473 | 78352 | 17:26635840 | 78357 | 17:26689830 | 78358 | 17:26694252 |
| 78359 | 17:26696213 | 78360 | 17:26697673 | 78362 | 17:26701612 | 7836 | 17:26731287 | 78365 | 17:26786597 |
| 78434 | 17:28547562 | 78459 | 17:29295556 | 78584 | 17:32536552 | 78587 | 17:32649823 | 78592 | 17:32740716 |
| 78597 | 17:32819809 | 78603 | 17:32962598 | 78617 | 17:33367107 | 7866 | 17:34481729 | 78680 | 17:34921588 |
| 78756 | 17:37414365 | 78774 | 17:38086662 | 78821 | 17:39604254 | 78835 | 17:39829382 | 78858 | 17:40028314 |
| 78866 | 17:40286179 | 78900 | 17:40901144 | 78909 | 17:41080474 | 78952 | 17:42386980 | 19026 | 17:44070161 |
| 79035 | 17:44280518 | 79050 | 17:44811665 | 79055 | 17:44886429 | 79088 | 17:45543757 | 79096 | 17:45774161 |
| 79102 | 17:45892895 | 79105 | 17:45908272 | 79147 | 17:46796152 | 79149 | 17:46809775 | 79150 | 17:46813078 |
| 79151 | 17:46817568 | 79152 | 17:46826871 | 79165 | 17:47298668 | 79168 | 17:47374048 | 79198 | 17:48075712 |
| 79217 | 17:48705395 | 79225 | 17:48883686 | 79261 | 17:49706199 | 79286 | 17:50270053 | 79354 | 17:51608281 |
| 79389 | 17:52115500 | 79391 | 17:52143477 | 79448 | 17:53925709 | 79467 | 17:54247909 | 79477 | 17:54494775 |
| 79546 | 17:56254970 | 79547 | 17:56259657 | 79577 | 17:57167393 | 79593 | 17:57507972 | 79621 | 17:57996907 |
| 79692 | 17:59257237 | 79708 | 17:59553144 | 79724 | 17:59967182 | 79763 | 17:60561298 | 79767 | 17:60653296 |
| 79776 | 17:60751860 | 79777 | 17:60776828 | 79791 | 17:60997432 | 79792 | 17:61002338 | 79793 | 17:61005235 |
| 79794 | 17:61005949 | 79799 | 17:61043189 | 79800 | 17:61068599 | 79804 | 17:61124602 | 79805 | 17:61125930 |
| 79806 | 17:61128578 | 79809 | 17:61149706 | 79811 | 17:61202835 | 79891 | 17:63194677 | 79894 | 17:63214725 |
| 79897 | 17:63260940 | 79924 | 17:63621804 | 79982 | 17:64851883 | 80057 | 17:66198895 | 80059 | 17:66227047 |
| 80104 | 17:67019380 | 80140 | 17:68128330 | 80158 | 17:68440573 | 80180 | 17:68951402 | 80183 | 17:68969488 |
| 80198 | 17:69224867 | 80234 | 17:70077612 | 80285 | 17:70987613 | 80306 | 17:71441658 | 80354 | 17:72302909 |
| 80450 | 18:1522517 | 80502 | 18:1802503 | 80508 | 18:1955689 | 80546 | 18:2744105 | 80574 | 18:3295457 |
| 80617 | 18:4333966 | 80629 | 18:4581539 | 80638 | 18:4763164 | 80660 | 18:5336542 | 80662 | 18:5358167 |
| 80679 | 18:5624880 | 80687 | 18:5808571 | 80698 | 18:6108723 | 80706 | 18:6166618 | 80715 | 18:6297029 |
| 80718 | 18:6328373 | 80801 | 18:7890552 | 80807 | 18:8137616 | 80811 | 18:8163590 | 80812 | 18:8166965 |
| 80813 | 18:8169505 | 80832 | 18:8512615 | 80833 | 18:8514080 | 80853 | 18:8977357 | 80855 | 18:9002587 |
| 80856 | 18:9003665 | 80857 | 18:9005002 | 80860 | 18:9032209 | 80867 | 18:9092871 | 80870 | 18:9135683 |
| 80881 | 18:9393342 | 80892 | 18:9513766 | 80943 | 18:10501656 | 80948 | 18:10593010 | 80951 | 18:10632938 |
| 80976 | 18:11155672 | 81045 | 18:12950981 | 81047 | 18:12967212 | 81076 | 18:13742613 | 81096 | 18:14219190 |
| 81124 | 18:15295023 | 81131 | 18:15355195 | 81156 | 18:16027392 | 81169 | 18:16320799 | 81178 | 18:16458903 |
| 81221 | 18:17381122 | 81234 | 18:17689118 | 81235 | 18:17739758 | 81305 | 18:19009400 | 81313 | 18:19119273 |
| 81333 | 18:19791460 | 81431 | 18:21965199 | 81444 | 18:22114555 | 81457 | 18:22311504 | 81475 | 18:22771950 |
| 81480 | 18:22863942 | 81493 | 18:23128319 | 81518 | 18:23726377 | 81540 | 18:24231814 | 81553 | 18:24599361 |
| 81590 | 18:25135273 | 81669 | 18:26830057 | 81689 | 18:27317835 | 81727 | 18:28307360 | 81772 | 18:29581458 |
| 81773 | 18:29587945 | 81783 | 18:29882243 | 81793 | 18:30199537 | 81805 | 18:30616085 | 81868 | 18:32286982 |
| 81873 | 18:32423923 | 81905 | 18:33102972 | 81907 | 18:33133073 | 81990 | 18:35092205 | 82005 | 18:35445465 |
| 82073 | 18:36962507 | 82091 | 18:37442129 | 82129 | 18:38169007 | 82139 | 18:38310010 | 82172 | 18:39170079 |
| 82266 | 18:41092657 | 82284 | 18:41476993 | 82310 | 18:42077536 | 82369 | 18:43146823 | 82379 | 18:43316323 |
| 82386 | 18:43394501 | 82412 | 18:43955990 | 82472 | 18:45795414 | 82478 | 18:45922293 | 82497 | 18:46310174 |
| 82503 | 18:46383891 | 82580 | 18:46784427 | 82591 | 18:46951310 | 82605 | 18:47321091 | 82699 | 18:49081014 |
| 82702 | 18:49135986 | 82724 | 18:49817333 | 82819 | 18:51867088 | 82926 | 18:54273698 | 82986 | 18:55259160 |
| 83048 | 18:56589786 | 83125 | 18:57434771 | 83138 | 18:57567593 | 83261 | 18:60011920 | 83313 | 18:61667250 |
| 83390 | 18:63188823 | 83451 | 18:64129214 | 83452 | 18:64134704 | 83491 | 18:64654290 | 83495 | 18:64731877 |
| 83505 | 18:64973481 | 83524 | 18:65148413 | 83569 | 18:65462851 | 83715 | 19:4421663 | 83817 | 19:6962233 |
| 83826 | 19:7152680 | 83862 | 19:7718388 | 83876 | 19:8025749 | 83881 | 19:8096180 | 83923 | 19:8635132 |
| 84010 | 19:10293568 | 84014 | 19:10327116 | 84026 | 19:10599040 | 84071 | 19:11761802 | 84072 | 19:11769174 |
| 84089 | 19:12304263 | 84170 | 19:13636619 | 84217 | 19:14667736 | 84289 | 19:16236117 | 84327 | 19:16897668 |
| 84339 | 19:17077928 | 84357 | 19:17402114 | 84369 | 19:17494959 | 84377 | 19:17756500 | 84400 | 19:18264868 |
| 84459 | 19:19573243 | 84465 | 19:19674513 | 84603 | 19:21820203 | 84607 | 19:21992770 | 84702 | 19:23904908 |
| 84739 | 19:24347524 | 84755 | 19:24595008 | 84785 | 19:24888212 | 84819 | 19:25338790 | 84846 | 19:25706233 |
| 84863 | 19:26003355 | 85007 | 19:28109982 | 85052 | 19:29119243 | 85053 | 19:29121068 | 85078 | 19:29541741 |
| 85111 | 19:30203261 | 85117 | 19:30384170 | 85124 | 19:30490687 | 85126 | 19:30515659 | 85144 | 19:30924489 |
| 85204 | 19:32369220 | 85387 | 19:35695286 | 85431 | 19:36295776 | 85435 | 19:36339138 | 85474 | 19:36999184 |
| 85492 | 19:37433519 | 85531 | 19:38678725 | 85532 | 19:38692719 | 85628 | 19:40886898 | 85633 | 19:40929680 |
| 85663 | 19:41630709 | 85664 | 19:41637084 | 85678 | 19:41804462 | 85753 | 19:43355438 | 85773 | 19:43531840 |
| 85794 | 19:44097163 | 85831 | 19:44865863 | 85905 | 19:46015589 | 85937 | 19:47051179 | 85964 | 19:47510334 |
| 85968 | 19:47580297 | 85999 | 19:48115274 | 86024 | 19:48601037 | 86035 | 19:48902647 | 86036 | 19:48931565 |
| 86071 | 19:49607062 | 86084 | 19:49887742 | 86208 | 19:51505417 | 86224 | 19:51685255 | 86249 | 19:52343433 |
| 86364 | 19:54493744 | 86401 | 19:55196487 | 86568 | 19:56726108 | 86587 | 19:57128485 | 86617 | 19:57698375 |
| 86625 | 19:58000641 | 86641 | 19:58231133 | 86813 | 19:61050416 | 86922 | 19:62757680 | 87001 | 20:973990 |
| 87030 | 20:1623518 | 87122 | 20:3090504 | 87167 | 20:4139957 | 87275 | 20:4685771 | 87328 | 20:4885163 |
| 87329 | 20:4922714 | 87330 | 20:4927232 | 87333 | 20:4936772 | 87428 | 20:5478088 | 87551 | 20:7762309 |
| 87571 | 20:8209150 | 87587 | 20:8561631 | 87601 | 20:8997165 | 87640 | 20:9653842 | 87783 | 20:12796928 |
| 87828 | 20:14014441 | 87898 | 20:16397316 | 87943 | 20:17594397 | 88015 | 20:19691235 | 88233 | 20:23492042 |
| 88295 | 20:24462304 | 88299 | 20:24489532 | 88400 | 20:27494213 | 88428 | 20:28090982 | 88464 | 20:28752172 |
| 88487 | 20:29353765 | 88516 | 20:30159496 | 88606 | 20:32897468 | 88712 | 20:35378617 | 88730 | 20:35746614 |
| 88754 | 20:36168903 | 88765 | 20:36410567 | 88793 | 20:37052070 | 88823 | 20:37587442 | 88896 | 20:39414225 |
| 88910 | 20:39546261 | 88982 | 20:41144615 | 89021 | 20:41891717 | 89030 | 20:42067151 | 89088 | 20:43711039 |
| 89200 | 20:46860182 | 89213 | 20:47186127 | 89219 | 20:47416742 | 89252 | 20:48508646 | 89386 | 20:52743029 |
| 89390 | 20:52943893 | 89391 | 20:52951946 | 89435 | 20:54078693 | 89459 | 20:54797651 | 89501 | 20:55920256 |
| 89547 | 20:56907438 | 89580 | 20:57704990 | 89603 | 20:58030736 | 89614 | 20:58246871 | 89619 | 20:58356797 |
| 89640 | 20:58731368 | 89642 | 20:58744546 | 89643 | 20:58745645 | 89644 | 20:58746207 | 89645 | 20:58747818 |
| 89646 | 20:58748682 | 89658 | 20:58932980 | 89659 | 20:58935094 | 89663 | 20:59004178 | 89666 | 20:59061779 |
| 89667 | 20:59062045 | 89674 | 20:59260188 | 89684 | 20:59558955 | 89695 | 20:59880963 | 89706 | 20:60058642 |
| 89712 | 20:60329519 | 89766 | 20:61949465 | 89781 | 20:62256568 | 89802 | 20:62677110 | 89850 | 20:63825165 |
| 89883 | 20:64360760 | 89988 | 20:66882747 | 89994 | 20:67014371 | 90009 | 20:67434867 | 90052 | 20:68158435 |
| 90064 | 20:68358778 | 90067 | 20:68381160 | 90101 | 20:68986868 | 90122 | 20:69449647 | 90149 | 20:70017030 |
| 90171 | 20:70615282 | 90299 | 21:1225541 | 90386 | 21:4129789 | 90390 | 21:4195328 | 90416 | 21:5081944 |
| 90422 | 21:5168513 | 90434 | 21:5505304 | 90441 | 21:5776117 | 90445 | 21:5799099 | 90449 | 21:5865604 |
| 90466 | 21:6422404 | 90500 | 21:7408212 | 90507 | 21:7585642 | 90558 | 21:8258222 | 90607 | 21:9489941 |
| 90625 | 21:9809810 | 90673 | 21:10967078 | 90681 | 21:11128214 | 90691 | 21:11412794 | 90733 | 21:12480366 |
| 90757 | 21:12890512 | 90783 | 21:13556460 | 90795 | 21:13850729 | 90814 | 21:14283158 | 90820 | 21:14450483 |
| 90838 | 21:14883665 | 90844 | 21:15011676 | 90955 | 21:17435585 | 90977 | 21:17918237 | 90983 | 21:18074742 |
| 91028 | 21:18934542 | 91038 | 21:19207258 | 91040 | 21:19228388 | 91042 | 21:19270043 | 91051 | 21:19367037 |
| 91086 | 21:19786899 | 91094 | 21:19865924 | 91121 | 21:20591184 | 91130 | 21:20646358 | 91220 | 21:22407987 |
| 91241 | 21:22944882 | 91282 | 21:24017992 | 91292 | 21:24359607 | 91349 | 21:26122546 | 91359 | 21:26345324 |
| 91365 | 21:26551273 | 91366 | 21:26568774 | 91378 | 21:26770078 | 91419 | 21:28090529 | 91451 | 21:29134066 |
| 91457 | 21:29367658 | 91458 | 21:29377639 | 91460 | 21:29411882 | 91490 | 21:30232035 | 91576 | 21:32380295 |
| 91580 | 21:32474563 | 91616 | 21:33229274 | 91662 | 21:33796466 | 91708 | 21:34496870 | 91781 | 21:35885347 |
| 91785 | 21:35973488 | 91802 | 21:36413785 | 91865 | 21:38466535 | 91868 | 21:38504846 | 91983 | 21:41529939 |
| 91990 | 21:41713492 | 92010 | 21:42131229 | 92100 | 21:44218636 | 92101 | 21:44266061 | 92104 | 21:44392496 |
| 92121 | 21:44681616 | 92122 | 21:44686161 | 92126 | 21:44715443 | 92130 | 21:44791184 | 92173 | 21:46307247 |
| 92174 | 21:46319841 | 92198 | 21:46784418 | 92299 | 21:48659647 | 92354 | 21:49876133 | 92365 | 21:50214540 |
| 92408 | 21:51612911 | 92464 | 21:53191938 | 92484 | 21:53761679 | 92513 | 21:54536881 | 92561 | 21:55372174 |
| 92563 | 21:55394291 | 92584 | 21:55724213 | 92598 | 21:56016789 | 92610 | 21:56129095 | 92628 | 21:56595068 |
| 92629 | 21:56609576 | 92630 | 21:56612935 | 92631 | 21:56613938 | 92632 | 21:56629591 | 92638 | 21:56817682 |
| 92639 | 21:56827233 | 92640 | 21:56835873 | 92641 | 21:56855414 | 92725 | 21:57245956 | 92790 | 21:58808829 |
| 92799 | 21:59077402 | 92811 | 21:59261689 | 92842 | 21:59841484 | 92856 | 21:59994711 | 92944 | 21:61845132 |
| 92947 | 21:61911883 | 92965 | 21:62176120 | 93011 | 21:63503056 | 93031 | 21:63983849 | 93033 | 21:64015058 |
| 93060 | 21:64546209 | 93125 | 21:65454701 | 93128 | 21:65507947 | 93165 | 21:66297320 | 93293 | 21:68995998 |
| 93474 | 22:3262622 | 93623 | 22:6202218 | 93632 | 22:6324139 | 93633 | 22:6332422 | 93693 | 22:7609231 |
| 93729 | 22:8491450 | 93766 | 22:9678468 | 93843 | 22:11157683 | 93862 | 22:11729652 | 93938 | 22:13149187 |
| 93946 | 22:13475890 | 94015 | 22:15394645 | 94026 | 22:15571331 | 94029 | 22:15621273 | 94030 | 22:15622509 |
| 94033 | 22:15666979 | 94043 | 22:15998599 | 94094 | 22:17137009 | 94106 | 22:17528025 | 94190 | 22:19523346 |
| 94236 | 22:20865658 | 94239 | 22:20892499 | 94240 | 22:20896013 | 94242 | 22:20900134 | 94248 | 22:21007069 |
| 94251 | 22:21064016 | 94261 | 22:21320700 | 94267 | 22:21445580 | 94279 | 22:21651340 | 94323 | 22:22860295 |
| 94327 | 22:22968191 | 94361 | 22:23904619 | 94386 | 22:24826985 | 94425 | 22:25962309 | 94516 | 22:28260377 |
| 94558 | 22:29032150 | 94569 | 22:29394147 | 94574 | 22:29509152 | 94575 | 22:29521016 | 94599 | 22:30171429 |
| 94614 | 22:30572708 | 94689 | 22:32709942 | 94699 | 22:32992978 | 94735 | 22:34025325 | 94738 | 22:34062706 |
| 94762 | 22:34833811 | 94770 | 22:34980123 | 94821 | 22:35930996 | 94832 | 22:36212273 | 94838 | 22:36427709 |
| 94897 | 22:37678461 | 94930 | 22:38553325 | 94957 | 22:39166570 | 94961 | 22:39195248 | 94982 | 22:39426381 |
| 94987 | 22:39550615 | 94998 | 22:39688034 | 95026 | 22:40340595 | 95073 | 22:41528657 | 95160 | 22:43561183 |
| 95169 | 22:43825654 | 95180 | 22:44046370 | 95244 | 22:45637290 | 95266 | 22:45983259 | 95343 | 22:47502907 |
| 95403 | 22:49140396 | 95406 | 22:49163015 | 95408 | 22:49233906 | 95450 | 22:50360859 | 95459 | 22:50587868 |
| 95461 | 22:50603931 | 95479 | 22:50938649 | 95519 | 22:52036264 | 95533 | 22:52343223 | 95551 | 22:52768115 |
| 95554 | 22:52841252 | 95575 | 22:53087988 | 95582 | 22:53268605 | 95650 | 22:54557160 | 95662 | 22:54685597 |
| 95702 | 22:55422111 | 95717 | 22:55662527 | 95742 | 22:55891155 | 95813 | 22:56887398 | 95816 | 22:56914900 |
| 95819 | 22:56956077 | 96032 | 23:149568 | 96113 | 23:2601798 | 96118 | 23:2653886 | 96121 | 23:2672734 |
| 96179 | 23:3852744 | 96189 | 23:3981656 | 96199 | 23:4118766 | 96251 | 23:5090449 | 96255 | 23:5153243 |
| 96280 | 23:5677678 | 96282 | 23:5716762 | 96311 | 23:6480089 | 96347 | 23:7174045 | 96353 | 23:7281266 |
| 96361 | 23:7443284 | 96593 | 23:12225865 | 96644 | 23:13147408 | 96654 | 23:13421583 | 96663 | 23:13674324 |
| 96671 | 23:13837654 | 96672 | 23:13841783 | 96674 | 23:13844745 | 96675 | 23:13850106 | 96676 | 23:13856428 |
| 96677 | 23:13860190 | 96678 | 23:13867299 | 96679 | 23:13870700 | 96680 | 23:13875916 | 96681 | 23:13885227 |
| 96708 | 23:14459570 | 96719 | 23:14657847 | 96734 | 23:14924427 | 96758 | 23:15231293 | 96802 | 23:15896256 |
| 96834 | 23:16779992 | 96854 | 23:17439199 | 96875 | 23:17872465 | 96879 | 23:17919886 | 96931 | 23:19271779 |
| 96944 | 23:19469822 | 96946 | 23:19474356 | 96988 | 23:20069380 | 97027 | 23:20827882 | 97070 | 23:21933675 |
| 97095 | 23:22572630 | 97114 | 23:23252435 | 97146 | 23:23919176 | 97176 | 23:24440913 | 97183 | 23:24589985 |
| 97237 | 23:25033379 | 97239 | 23:25047940 | 97376 | 23:27210848 | 97398 | 23:27410296 | 97713 | 23:31798120 |
| 97740 | 23:32277024 | 97758 | 23:32470121 | 97771 | 23:32757047 | 97785 | 23:33017631 | 97799 | 23:33301724 |
| 97830 | 23:34183106 | 97879 | 23:35808678 | 97919 | 23:36940417 | 97987 | 23:38818865 | 97990 | 23:38876101 |
| 98006 | 23:39270706 | 98145 | 23:40884084 | 98169 | 23:41183712 | 98196 | 23:41831694 | 98206 | 23:42044741 |
| 98238 | 23:42772846 | 98243 | 23:42890423 | 98262 | 23:43295377 | 98268 | 23:43447515 | 98302 | 23:44180408 |
| 98311 | 23:44267686 | 98338 | 23:44807132 | 98340 | 23:44830102 | 98351 | 23:45020916 | 98363 | 23:45143240 |
| 98365 | 23:45154456 | 98366 | 23:45154585 | 98367 | 23:45154695 | 98369 | 23:45170649 | 98370 | 23:45174122 |
| 98371 | 23:45194932 | 98372 | 23:45209920 | 98415 | 23:46222052 | 98487 | 23:47700808 | 98491 | 23:47790490 |
| 98613 | 23:49822048 | 98646 | 23:50347036 | 98647 | 23:50349795 | 98648 | 23:50361478 | 98695 | 23:51211166 |
| 98758 | 23:52291892 | 98780 | 24:465578 | 98795 | 24:844195 | 98914 | 24:3199425 | 99033 | 24:6106333 |
| 99089 | 24:7522593 | 99139 | 24:8885668 | 99310 | 24:11057073 | 99312 | 24:11077021 | 99344 | 24:11884545 |
| 99368 | 24:12540583 | 99376 | 24:12720792 | 99407 | 24:13477647 | 99436 | 24:14392789 | 99449 | 24:14713059 |
| 99481 | 24:15784201 | 99482 | 24:15793449 | 99520 | 24:16988712 | 99542 | 24:17540200 | 99608 | 24:19628750 |
| 99633 | 24:20394869 | 99814 | 24:23537983 | 99838 | 24:24399016 | 99917 | 24:26088773 | 99969 | 24:27563741 |
| 100022 | 24:28948556 | 100051 | 24:29641974 | 100053 | 24:29754026 | 100083 | 24:30348689 | 100125 | 24:31468489 |
| 100149 | 24:31977640 | 100204 | 24:33038149 | 100247 | 24:34170378 | 100262 | 24:34562426 | 100316 | 24:35736460 |
| 100427 | 24:39087533 | 100457 | 24:39886402 | 100506 | 24:40530911 | 100517 | 24:40707643 | 100546 | 24:41182679 |
| 100560 | 24:41739524 | 100603 | 24:42667191 | 100619 | 24:42900584 | 100623 | 24:43047337 | 100646 | 24:44068866 |
| 100667 | 24:44457109 | 100702 | 24:45155232 | 100708 | 24:45303639 | 100817 | 24:46988217 | 100842 | 24:47695031 |
| 100848 | 24:47858503 | 100861 | 24:48130487 | 100927 | 24:49987049 | 100960 | 24:50907651 | 100967 | 24:51121771 |
| 100974 | 24:51293921 | 101015 | 24:52055045 | 101040 | 24:52603746 | 101042 | 24:52612789 | 101103 | 24:54184345 |
| 101132 | 24:54726088 | 101133 | 24:54737023 | 101135 | 24:54745793 | 101174 | 24:55654866 | 101209 | 24:56364015 |
| 101214 | 24:56468941 | 101217 | 24:56488437 | 101221 | 24:56542836 | 101264 | 24:57462922 | 101329 | 24:58647028 |
| 101359 | 24:59350759 | 101409 | 24:60371452 | 101438 | 24:60938433 | 101480 | 24:61694231 | 101482 | 24:61715423 |
| 101486 | 24:61776900 | 101548 | 25:1123694 | 101554 | 25:1211629 | 101568 | 25:1368623 | 101741 | 25:2890918 |
| 101757 | 25:3291302 | 101761 | 25:3373790 | 101762 | 25:3388313 | 101779 | 25:3563948 | 101807 | 25:4175576 |
| 101833 | 25:4621429 | 101909 | 25:5758755 | 101916 | 25:5934892 | 101922 | 25:6041488 | 101970 | 25:7127243 |
| 102009 | 25:7910528 | 102017 | 25:8111580 | 102058 | 25:9294790 | 102089 | 25:9965727 | 102096 | 25:10068090 |
| 102163 | 25:11303404 | 102165 | 25:11364705 | 102181 | 25:11635729 | 102182 | 25:11657253 | 102214 | 25:12395830 |
| 102217 | 25:12420213 | 102227 | 25:12757945 | 102253 | 25:13410180 | 102261 | 25:13589337 | 102275 | 25:14025698 |
| 102289 | 25:14337887 | 102372 | 25:16006571 | 102395 | 25:16397106 | 102396 | 25:16427094 | 102524 | 25:19554407 |
| 102537 | 25:19935329 | 102562 | 25:20762754 | 102600 | 25:21483496 | 102602 | 25:21510337 | 102641 | 25:22365508 |
| 102653 | 25:22668990 | 102699 | 25:23839896 | 102714 | 25:24079936 | 102736 | 25:24542279 | 102742 | 25:24617636 |
| 102767 | 25:25119747 | 102769 | 25:25147993 | 102791 | 25:25378579 | 102816 | 25:26015769 | 102932 | 25:28026271 |
| 102975 | 25:29198572 | 102994 | 25:29627243 | 103031 | 25:30198863 | 103080 | 25:31339867 | 103118 | 25:32018635 |
| 103199 | 25:33663856 | 103229 | 25:34356818 | 103305 | 25:35625080 | 103338 | 25:36046363 | 103342 | 25:36137423 |
| 103378 | 25:36442406 | 103457 | 25:37592711 | 103460 | 25:37631841 | 103479 | 25:38076102 | 103536 | 25:39097677 |
| 103596 | 25:40251086 | 103616 | 25:40630329 | 103683 | 25:41733621 | 103706 | 26:19998 | 103766 | 26:2270743 |
| 103818 | 26:3188174 | 103819 | 26:3191375 | 103829 | 26:3361204 | 103909 | 26:5551721 | 104006 | 26:8275246 |
| 104020 | 26:8585213 | 104105 | 26:10587007 | 104135 | 26:11460715 | 104155 | 26:11751995 | 104195 | 26:13034814 |
| 104284 | 26:14982249 | 104298 | 26:15453105 | 104410 | 26:18118254 | 104413 | 26:18261377 | 104460 | 26:19187481 |
| 104469 | 26:19395441 | 104471 | 26:19411437 | 104493 | 26:19971593 | 104520 | 26:20855927 | 104538 | 26:21270336 |
| 104539 | 26:21271111 | 104540 | 26:21272246 | 104542 | 26:21276672 | 104554 | 26:21609152 | 104556 | 26:21638374 |
| 104557 | 26:21659298 | 104575 | 26:22193698 | 104576 | 26:22197941 | 104651 | 26:24350779 | 104659 | 26:24607752 |
| 104686 | 26:24921440 | 104693 | 26:25131752 | 104762 | 26:26169541 | 104768 | 26:26273875 | 104774 | 26:26426669 |
| 104788 | 26:26770718 | 104860 | 26:28504622 | 104867 | 26:28761933 | 104935 | 26:30753391 | 104952 | 26:31231421 |
| 104953 | 26:31240168 | 105002 | 26:32486347 | 105010 | 26:32645140 | 105037 | 26:33152552 | 105049 | 26:33514228 |
| 105055 | 26:33591046 | 105060 | 26:33741604 | 105081 | 26:34086479 | 105174 | 26:36209430 | 105228 | 26:37411119 |
| 105255 | 26:38327739 | 105259 | 26:38422867 | 105266 | 26:38582767 | 105287 | 26:39039410 | 105319 | 26:39645163 |
| 105321 | 26:39706475 | 105436 | 26:42682884 | 105480 | 26:43924743 | 105484 | 26:44008043 | 105557 | 26:45531818 |
| 105583 | 26:46019051 | 105611 | 26:46544547 | 105633 | 26:47031869 | 105741 | 26:49412031 | 105763 | 26:49860726 |
| 105823 | 26:51197832 | 105827 | 26:51273644 | 105828 | 26:51339616 | 105832 | 26:51408594 | 105941 | 27:2266510 |
| 105949 | 27:2424175 | 106008 | 27:3926602 | 106025 | 27:4205889 | 106087 | 27:5556009 | 106166 | 27:7956657 |
| 106176 | 27:8174752 | 106200 | 27:8748506 | 106216 | 27:9098489 | 106217 | 27:9098570 | 106218 | 27:9098663 |
| 106219 | 27:9099087 | 106220 | 27:9099279 | 106221 | 27:9099292 | 106222 | 27:9099380 | 106223 | 27:9099465 |
| 106224 | 27:9099696 | 106225 | 27:9099832 | 106226 | 27:9104046 | 106228 | 27:9106497 | 106230 | 27:9110724 |
| 106231 | 27:9116774 | 106232 | 27:9117417 | 106233 | 27:9117873 | 106234 | 27:9117874 | 106235 | 27:9118114 |
| 106236 | 27:9118136 | 106237 | 27:9118300 | 106240 | 27:9149222 | 106241 | 27:9151257 | 106256 | 27:9578023 |
| 106335 | 27:12004591 | 106383 | 27:13309317 | 106461 | 27:14313738 | 106474 | 27:14529862 | 106519 | 27:15481603 |
| 106554 | 27:16106255 | 106656 | 27:18041339 | 106668 | 27:18444933 | 106677 | 27:18618418 | 106687 | 27:18831735 |
| 106688 | 27:18844548 | 106691 | 27:18895158 | 106692 | 27:18916416 | 106693 | 27:18921032 | 106756 | 27:20229425 |
| 106790 | 27:21326986 | 106801 | 27:21699196 | 106805 | 27:21744178 | 106817 | 27:21948844 | 106848 | 27:22823774 |
| 106884 | 27:23610622 | 106908 | 27:24137880 | 107014 | 27:26432887 | 107082 | 27:27644914 | 107095 | 27:27964245 |
| 107202 | 27:30418455 | 107334 | 27:32927582 | 107343 | 27:33025196 | 107408 | 27:34899643 | 107448 | 27:35233200 |
| 107456 | 27:35414868 | 107653 | 27:39221884 | 107712 | 27:40883338 | 107746 | 27:41747888 | 107759 | 27:42126889 |
| 107801 | 27:43079592 | 107803 | 27:43088850 | 107843 | 27:43939855 | 107846 | 27:43979622 | 107847 | 27:43983990 |
| 107854 | 27:44107624 | 107862 | 27:44229578 | 107939 | 28:1012326 | 107958 | 28:1304486 | 107974 | 28:1564870 |
| 108000 | 28:2124177 | 108040 | 28:3650698 | 108041 | 28:3666554 | 108059 | 28:4687863 | 108061 | 28:4710151 |
| 108084 | 28:5241897 | 108148 | 28:6765529 | 108153 | 28:6864730 | 108232 | 28:8673885 | 108245 | 28:9022551 |
| 108246 | 28:9025349 | 108267 | 28:9565874 | 108279 | 28:9801762 | 108289 | 28:9992581 | 108310 | 28:10520846 |
| 108311 | 28:10565818 | 108389 | 28:12680409 | 108396 | 28:12751017 | 108476 | 28:14911107 | 108581 | 28:17140257 |
| 108632 | 28:18044696 | 108660 | 28:18836541 | 108739 | 28:20933740 | 108741 | 28:20941251 | 108783 | 28:22351391 |
| 108826 | 28:23326980 | 108859 | 28:24154609 | 108866 | 28:24223951 | 108903 | 28:25048959 | 108912 | 28:25216858 |
| 109001 | 28:26844276 | 109007 | 28:27009355 | 109043 | 28:27784145 | 109052 | 28:27909599 | 109054 | 28:27910886 |
| 109073 | 28:28015394 | 109215 | 28:31286284 | 109245 | 28:32003701 | 109325 | 28:33230710 | 109348 | 28:33703563 |
| 109406 | 28:34803927 | 109413 | 28:34924460 | 109417 | 28:34965171 | 109472 | 28:36153178 | 109522 | 28:37465397 |
| 109570 | 28:38512629 | 109579 | 28:38669301 | 109622 | 28:39347343 | 109640 | 28:39934529 | 109726 | 28:40743505 |
| 109728 | 28:40802700 | 109729 | 28:40815314 | 109735 | 28:40867971 | 109741 | 28:40986070 | 109745 | 28:41069948 |
| 109774 | 28:41574166 | 109825 | 28:42451978 | 109871 | 28:43255478 | 109915 | 28:44119755 | 109971 | 28:45223304 |
| 110037 | 29:1129729 | 110066 | 29:1630821 | 110088 | 29:2215375 | 110115 | 29:2538458 | 110120 | 29:2640739 |
| 110159 | 29:3485853 | 110162 | 29:3535737 | 110211 | 29:4767448 | 110325 | 29:8136563 | 110346 | 29:8758233 |
| 110486 | 29:11888737 | 110531 | 29:12717025 | 110539 | 29:12922913 | 110611 | 29:14988640 | 110619 | 29:15140865 |
| 110686 | 29:16917842 | 110688 | 29:16949756 | 110701 | 29:17144041 | 110705 | 29:17304932 | 110731 | 29:18241209 |
| 110749 | 29:19246346 | 110842 | 29:21593668 | 110893 | 29:23085229 | 110896 | 29:23104998 | 110900 | 29:23212423 |
| 110941 | 29:24050623 | 111030 | 29:26373975 | 111053 | 29:27005732 | 111066 | 29:27532899 | 111074 | 29:27658327 |
| 111146 | 29:29021609 | 111151 | 29:29269330 | 111156 | 29:29356340 | 111204 | 29:29930474 | 111235 | 29:30673349 |
| 111259 | 29:31276001 | 111275 | 29:31521382 | 111281 | 29:31682726 | 111359 | 29:33581781 | 111361 | 29:33604607 |
| 111377 | 29:33936893 | 111379 | 29:33963898 | 111395 | 29:34390629 | 111404 | 29:34569090 | 111415 | 29:34694545 |
| 111459 | 29:35661091 | 111465 | 29:35722342 | 111509 | 29:36453498 | 111527 | 29:36872914 | 111613 | 29:39811611 |
| 111614 | 29:39822689 | 111712 | 29:41607581 | 111724 | 29:41814129 | 111745 | 29:42165191 | 111770 | 29:42669048 |
| 111788 | 29:43153261 | 111839 | 29:43881785 | 111856 | 29:44096512 | 111873 | 29:44366521 | 111948 | 29:46176719 |
| 111954 | 29:46281096 | 112022 | 29:47700163 | 112024 | 29:47731596 | 112148 | 29:50449063; | | |

and
the sites of the fifth marker panel are shown in the following table:

| NO. | Position | NO. | Position | NO. | Position | NO. | Position | NO. | Position |
|---|---|---|---|---|---|---|---|---|---|
| 36 | 1:1247889 | 37 | 1:1248205 | 38 | 1:1248699 | 39 | 1:1252437 | 46 | 1:1417011 |
| 47 | 1:1438871 | 48 | 1:1439812 | 51 | 1:1473953 | 52 | 1:1474233 | 53 | 1:1505639 |
| 54 | 1:1517594 | 55 | 1:1518088 | 56 | 1:1523007 | 57 | 1:1524793 | 58 | 1:1540344 |
| 59 | 1:1540422 | 60 | 1:1540458 | 61 | 1:1540555 | 119 | 1:2769164 | 139 | 1:3245141 |
| 144 | 1:3303269 | 169 | 1:3679697 | 170 | 1:3679721 | 171 | 1:3687775 | 185 | 1:3908027 |
| 207 | 1:4250046 | 209 | 1:4276065 | 400 | 1:8645661 | 446 | 1:9625272 | 607 | 1:13657759 |
| 715 | 1:16486738 | 725 | 1:16754495 | 738 | 1:17263605 | 750 | 1:17537664 | 784 | 1:18789066 |
| 935 | 1:23001427 | 965 | 1:23946108 | 991 | 1:24758865 | 993 | 1:24782983 | 1041 | 1:26105452 |
| 1093 | 1:27491647 | 1117 | 1:28566525 | 1118 | 1:28612680 | 1152 | 1:29520198 | 1205 | 1:31082779 |
| 1284 | 1:33805826 | 1294 | 1:33964232 | 1471 | 1:38630217 | 1603 | 1:42156367 | 1607 | 1:42205645 |
| 1646 | 1:43258942 | 1650 | 1:43390633 | 1777 | 1:46016435 | 1879 | 1:49422581 | 1953 | 1:51785197 |
| 1964 | 1:52229363 | 1967 | 1:52281229 | 2171 | 1:57147119 | 2172 | 1:57180246 | 2296 | 1:60247154 |
| 2297 | 1:60275851 | 2332 | 1:61100002 | 2339 | 1:61219155 | 2362 | 1:61796684 | 2462 | 1:64193131 |
| 2469 | 1:64272382 | 2530 | 1:65583709 | 2532 | 1:65625274 | 2535 | 1:65763047 | 2669 | 1:69022641 |
| 2694 | 1:69479795 | 2715 | 1:69942383 | 2774 | 1:70823693 | 2916 | 1:74460838 | 3081 | 1:78554220 |
| 3121 | 1:79513248 | 3351 | 1:85398326 | 3352 | 1:85456698 | 3354 | 1:85501954 | 3382 | 1:86229426 |
| 3387 | 1:86366405 | 3411 | 1:87047388 | 3436 | 1:87762016 | 3496 | 1:89623272 | 3548 | 1:90545947 |
| 3594 | 1:91706588 | 3654 | 1:93372952 | 3656 | 1:93477242 | 3826 | 1:97778617 | 3827 | 1:97802384 |
| 3832 | 1:97878133 | 3860 | 1:98721777 | 3895 | 1:99586706 | 4190 | 1:108556096 | 4194 | 1:108577099 |
| 4350 | 1:112307236 | 4383 | 1:112837296 | 4428 | 1:113860732 | 4434 | 1:114102926 | 4466 | 1:114828912 |
| 4467 | 1:114837165 | 4530 | 1:116786787 | 4560 | 1:117406892 | 4601 | 1:118129247 | 4619 | 1:118286166 |
| 4638 | 1:118603687 | 4640 | 1:118638525 | 4644 | 1:118785100 | 4645 | 1:118808555 | 4676 | 1:119387495 |
| 4706 | 1:120231880 | 4757 | 1:121536001 | 4852 | 1:124087395 | 4901 | 1:125202968 | 4921 | 1:125609019 |
| 4991 | 1:127321191 | 5033 | 1:128301452 | 5034 | 1:128336208 | 5132 | 1:130745923 | 5143 | 1:131052225 |
| 5212 | 1:131617330 | 5252 | 1:132852921 | 5255 | 1:132924609 | 5257 | 1:132963363 | 5326 | 1:134972795 |
| 5386 | 1:135785234 | 5387 | 1:135785949 | 5388 | 1:135787843 | 5389 | 1:135788916 | 5390 | 1:135789433 |
| 5391 | 1:135791112 | 5448 | 1:137501805 | 5548 | 1:140642688 | 5607 | 1:141638716 | 5670 | 1:142753326 |
| 5736 | 1:143879207 | 5833 | 1:145815334 | 5857 | 1:146000862 | 5863 | 1:146077457 | 5868 | 1:146242466 |
| 5876 | 1:146437585 | 5882 | 1:146612774 | 5891 | 1:146975346 | 5965 | 1:148149815 | 6115 | 1:150888684 |
| 6274 | 1:153150228 | 6320 | 1:153989230 | 6340 | 1:154314444 | 6342 | 1:154371107 | 6343 | 1:154385540 |
| 6547 | 2:682101 | 6574 | 2:1161894 | 6626 | 2:2113211 | 6636 | 2:2405667 | 6699 | 2:4200142 |
| 6781 | 2:5668859 | 6810 | 2:6248437 | 6811 | 2:6253061 | 6813 | 2:6269804 | 6815 | 2:6275413 |
| 6834 | 2:6717203 | 6835 | 2:6717548 | 6836 | 2:6732409 | 6837 | 2:6732955 | 6873 | 2:7379901 |
| 6888 | 2:7706163 | 6911 | 2:8334857 | 6932 | 2:8969249 | 7019 | 2:11681348 | 7024 | 2:11775775 |
| 7049 | 2:12802862 | 7070 | 2:13346155 | 7119 | 2:14632486 | 7120 | 2:14676143 | 7125 | 2:14743753 |
| 7148 | 2:15330545 | 7152 | 2:15479415 | 7159 | 2:15620578 | 7175 | 2:15995516 | 7188 | 2:16261766 |
| 7244 | 2:17777834 | 7245 | 2:17826748 | 7280 | 2:18272965 | 7294 | 2:18380242 | 7300 | 2:18552906 |
| 7374 | 2:20073035 | 7420 | 2:21391926 | 7484 | 2:23026213 | 7579 | 2:24732559 | 7750 | 2:28419342 |
| 7751 | 2:28419366 | 7752 | 2:28421072 | 8028 | 2:35888381 | 8230 | 2:41375205 | 8293 | 2:42660415 |
| 8319 | 2:43375192 | 8449 | 2:46777670 | 8450 | 2:46782605 | 8452 | 2:46797429 | 8466 | 2:47070734 |
| 8467 | 2:47100164 | 8549 | 2:49214428 | 8560 | 2:49760042 | 8561 | 2:49760735 | 8735 | 2:54491736 |
| 8791 | 2:55960682 | 8792 | 2:55979085 | 9079 | 2:63568686 | 9094 | 2:64064953 | 9161 | 2:65602756 |
| 9288 | 2:69830690 | 9347 | 2:71285767 | 9373 | 2:72193932 | 9374 | 2:72216636 | 9516 | 2:76002159 |
| 9530 | 2:76376460 | 9555 | 2:76976290 | 9786 | 2:83877880 | 9791 | 2:84089700 | 10121 | 2:91931085 |
| 10139 | 2:92377009 | 10180 | 2:93321855 | 10182 | 2:93326125 | 10183 | 2:93326273 | 10184 | 2:93327304 |
| 10185 | 2:93353835 | 10186 | 2:93353877 | 10187 | 2:93354446 | 10188 | 2:93354619 | 10189 | 2:93354804 |
| 10190 | 2:93355262 | 10191 | 2:93355271 | 10192 | 2:93356138 | 10193 | 2:93356208 | 10194 | 2:93356223 |
| 10195 | 2:93366311 | 10248 | 2:94636270 | 10286 | 2:95449694 | 10305 | 2:95979209 | 10312 | 2:96139223 |
| 10401 | 2:98337087 | 10409 | 2:98421675 | 10413 | 2:98461459 | 10417 | 2:98536760 | 10466 | 2:99940359 |
| 10605 | 2:103776260 | 10655 | 2:105009833 | 10686 | 2:105635824 | 10694 | 2:105828123 | 10712 | 2:106230731 |
| 10781 | 2:107728454 | 10782 | 2:107728517 | 10783 | 2:107729520 | 10784 | 2:107730636 | 10785 | 2:107735095 |
| 10786 | 2:107735449 | 10787 | 2:107737533 | 10789 | 2:107742839 | 10800 | 2:107971542 | 10846 | 2:109019023 |
| 10929 | 2:111354781 | 10934 | 2:111635526 | 10954 | 2:112098945 | 10956 | 2:112105210 | 10975 | 2:112632558 |
| 10978 | 2:112654694 | 11038 | 2:113914830 | 11099 | 2:115533946 | 11303 | 2:120430113 | 11405 | 2:123285701 |
| 11444 | 2:124279927 | 11460 | 2:124841893 | 11684 | 2:128718214 | 11813 | 2:131557054 | 11822 | 2:131862099 |
| 11852 | 2:132351992 | 11940 | 2:133852858 | 11943 | 2:133878448 | 11959 | 2:134115936 | 11960 | 2:134172533 |
| 12004 | 2:134750704 | 12059 | 2:135525484 | 12143 | 3:950659 | 12145 | 3:954886 | 12187 | 3:1728119 |
| 12274 | 3:3596795 | 12343 | 3:4727841 | 12452 | 3:6832814 | 12454 | 3:6841596 | 12456 | 3:6848201 |
| 12457 | 3:6866175 | 12458 | 3:6867557 | 12459 | 3:6867867 | 12460 | 3:6885163 | 12533 | 3:8025174 |
| 12734 | 3:11445314 | 12790 | 3:12934994 | 12856 | 3:14226434 | 12920 | 3:15616501 | 12948 | 3:15994930 |
| 13048 | 3:18771436 | 13049 | 3:18775018 | 13138 | 3:21163145 | 13156 | 3:21764453 | 13190 | 3:22332383 |
| 13191 | 3:22334033 | 13192 | 3:22339164 | 13193 | 3:22340631 | 13194 | 3:22343298 | 13357 | 3:26160439 |
| 13470 | 3:27968845 | 13475 | 3:28047336 | 13527 | 3:29246450 | 13584 | 3:30235042 | 13600 | 3:30605190 |
| 13623 | 3:31149250 | 13660 | 3:31948925 | 13662 | 3:31976577 | 13736 | 3:32970665 | 13765 | 3:33258971 |
| 14049 | 3:40032182 | 14051 | 3:40065534 | 14053 | 3:40120645 | 14188 | 3:43541601 | 14331 | 3:47791771 |
| 14354 | 3:48191758 | 14355 | 3:48234094 | 14392 | 3:49000989 | 14416 | 3:49459128 | 14422 | 3:49526251 |
| 14428 | 3:49574985 | 14483 | 3:49988687 | 14504 | 3:50656944 | 14581 | 3:52783346 | 14661 | 3:55239885 |
| 14665 | 3:55330131 | 14835 | 3:60375726 | 14845 | 3:60575455 | 14850 | 3:60636353 | 14868 | 3:60938213 |
| 14958 | 3:63728694 | 14960 | 3:63776877 | 14965 | 3:63861865 | 14966 | 3:63887795 | 14968 | 3:63950495 |
| 14969 | 3:63955495 | 14970 | 3:63976119 | 14973 | 3:64020621 | 14975 | 3:64058308 | 14983 | 3:64239387 |
| 14988 | 3:64525583 | 14992 | 3:64697961 | 15033 | 3:65670943 | 15044 | 3:66042478 | 15049 | 3:66148244 |
| 15107 | 3:67465318 | 15130 | 3:67947626 | 15131 | 3:67978806 | 15150 | 3:68443615 | 15175 | 3:69034450 |
| 15230 | 3:70652398 | 15234 | 3:70860935 | 15243 | 3:71230722 | 15285 | 3:72930619 | 15393 | 3:76153357 |
| 15442 | 3:77599781 | 15443 | 3:77635657 | 15446 | 3:77784239 | 15495 | 3:79128870 | 15498 | 3:79224313 |
| 15538 | 3:80529583 | 15563 | 3:81048556 | 15605 | 3:82166845 | 15625 | 3:82581418 | 15684 | 3:84280062 |
| 15939 | 3:89312804 | 15941 | 3:89315922 | 16034 | 3:91258808 | 16253 | 3:96974610 | 16259 | 3:97049079 |
| 16287 | 3:97630051 | 16414 | 3:100563623 | 16430 | 3:100751713 | 16431 | 3:100782663 | 16443 | 3:101112794 |
| 16446 | 3:101150016 | 16537 | 3:102883747 | 16541 | 3:102931082 | 16598 | 3:104333611 | 16602 | 3:104484207 |
| 16605 | 3:104506752 | 16607 | 3:104541890 | 16609 | 3:104556518 | 16628 | 3:104869949 | 16646 | 3:105209686 |
| 16654 | 3:105292781 | 16714 | 3:105965567 | 16715 | 3:105973059 | 16718 | 3:106026621 | 16719 | 3:106042336 |
| 16740 | 3:106492826 | 16750 | 3:106599058 | 16901 | 3:109689309 | 17056 | 3:113011961 | 17068 | 3:113301217 |
| 17115 | 3:113990093 | 17186 | 3:114897383 | 17380 | 3:117882488 | 17384 | 3:117909372 | 17401 | 3:118223118 |
| 17402 | 3:118253078 | 17639 | 4:4003154 | 17644 | 4:4149740 | 17697 | 4:5004514 | 17705 | 4:5165078 |
| 17706 | 4:5173632 | 17740 | 4:5747787 | 17752 | 4:6130688 | 17761 | 4:6330228 | 17968 | 4:10578187 |
| 17969 | 4:10580814 | 18198 | 4:16670558 | 18273 | 4:18595000 | 18276 | 4:18619515 | 18318 | 4:19200647 |
| 18378 | 4:20303118 | 18635 | 4:26901086 | 18636 | 4:26934291 | 18820 | 4:32010653 | 18843 | 4:32768824 |
| 18949 | 4:35971843 | 19100 | 4:40158341 | 19134 | 4:40946237 | 19168 | 4:41815635 | 19173 | 4:42048079 |
| 19208 | 4:43120197 | 19214 | 4:43203876 | 19250 | 4:44121669 | 19285 | 4:45161683 | 19356 | 4:47190294 |
| 19377 | 4:47463756 | 19400 | 4:48100380 | 19424 | 4:48952233 | 19466 | 4:49461342 | 19477 | 4:49813182 |
| 19478 | 4:49842196 | 19480 | 4:49888592 | 19866 | 4:61781138 | 19888 | 4:62276469 | 19891 | 4:62357256 |
| 19931 | 4:63512286 | 19965 | 4:64527362 | 19982 | 4:64979970 | 20028 | 4:66103436 | 20075 | 4:67374218 |
| 20136 | 4:69236841 | 20289 | 4:73424213 | 20513 | 4:78984643 | 20553 | 4:80105286 | 20554 | 4:80107022 |
| 20555 | 4:80107183 | 20556 | 4:80107305 | 20557 | 4:80107320 | 20558 | 4:80107386 | 20559 | 4:80107434 |
| 20560 | 4:80107694 | 20561 | 4:80107717 | 20562 | 4:80107759 | 20674 | 4:82773442 | 20681 | 4:82863329 |
| 20749 | 4:85034221 | 20779 | 4:85832979 | 20780 | 4:85866052 | 20788 | 4:85968886 | 20834 | 4:87033808 |
| 20882 | 4:88417629 | 20886 | 4:88515168 | 20903 | 4:89049127 | 21046 | 4:92398977 | 21050 | 4:92448883 |
| 21051 | 4:92451008 | 21052 | 4:92451165 | 21109 | 4:93517701 | 21117 | 4:93652019 | 21185 | 4:95523178 |
| 21553 | 4:100762424 | 21603 | 4:101689426 | 21683 | 4:103739455 | 21711 | 4:104521615 | 21718 | 4:104699947 |
| 21818 | 4:106683233 | 21824 | 4:106777370 | 21836 | 4:106986917 | 21886 | 4:107625627 | 21968 | 4:109840544 |
| 22030 | 4:111174187 | 22078 | 4:111934020 | 22116 | 4:112499124 | 22123 | 4:112606237 | 22134 | 4:112692746 |
| 22137 | 4:112719377 | 22140 | 4:112741810 | 22149 | 4:112797073 | 22180 | 4:113503432 | 22250 | 4:114401519 |
| 22253 | 4:114443077 | 22274 | 4:114788184 | 22278 | 4:114812550 | 22312 | 4:115192347 | 22318 | 4:115316340 |
| 22319 | 4:115317642 | 22354 | 4:116067653 | 22376 | 4:116420553 | 22394 | 4:116749463 | 22406 | 4:116987390 |
| 22513 | 4:119160825 | 22525 | 4:119536985 | 22581 | 5:882265 | 22670 | 5:2514974 | 22693 | 5:3086065 |
| 22707 | 5:3684231 | 22731 | 5:4371208 | 22740 | 5:4517412 | 22761 | 5:5025515 | 22776 | 5:5256216 |
| 22899 | 5:8017094 | 22935 | 5:8963886 | 22937 | 5:9058312 | 22939 | 5:9081371 | 22941 | 5:9110521 |
| 22974 | 5:9917938 | 23184 | 5:14901254 | 23203 | 5:15246344 | 23335 | 5:18879061 | 23340 | 5:18957429 |
| 23448 | 5:21803211 | 23511 | 5:22947860 | 23513 | 5:23010158 | 23563 | 5:23986445 | 23619 | 5:25424207 |
| 23628 | 5:25663032 | 23683 | 5:27040938 | 23685 | 5:27062257 | 23767 | 5:28682732 | 23795 | 5:29481439 |
| 23894 | 5:31935957 | 23904 | 5:32259633 | 23908 | 5:32334842 | 23922 | 5:32649736 | 23951 | 5:33311326 |
| 24015 | 5:35004932 | 24046 | 5:36022885 | 24051 | 5:36113810 | 24053 | 5:36193854 | 24092 | 5:37455345 |
| 24105 | 5:37749836 | 24243 | 5:41293748 | 24244 | 5:41337074 | 24247 | 5:41451300 | 24254 | 5:41682610 |
| 24265 | 5:41958597 | 24268 | 5:42010622 | 24275 | 5:42382038 | 24295 | 5:42883310 | 24301 | 5:43024524 |
| 24312 | 5:43371061 | 24352 | 5:44118721 | 24387 | 5:45222817 | 24450 | 5:46743902 | 24683 | 5:52698528 |
| 24690 | 5:53009069 | 25020 | 5:61964739 | 25024 | 5:62044690 | 25041 | 5:62542053 | 25049 | 5:62915639 |
| 25350 | 5:69917738 | 25398 | 5:71197728 | 25467 | 5:72417962 | 25469 | 5:72465277 | 25527 | 5:74079648 |
| 25639 | 5:76766433 | 25642 | 5:76818912 | 25644 | 5:76890153 | 25645 | 5:76972053 | 25757 | 5:80114008 |
| 25760 | 5:80235852 | 25784 | 5:80982109 | 25787 | 5:81116016 | 25814 | 5:81935764 | 25875 | 5:83029733 |
| 26074 | 5:84527341 | 26206 | 5:87607418 | 26217 | 5:87838676 | 26304 | 5:89147995 | 26364 | 5:90614584 |
| 26395 | 5:91363881 | 26508 | 5:93490294 | 26558 | 5:94637080 | 26603 | 5:95645793 | 26605 | 5:95684692 |
| 26607 | 5:95699816 | 26610 | 5:95747447 | 26611 | 5:95748288 | 26612 | 5:95749996 | 26763 | 5:98601044 |
| 26855 | 5:100383163 | 26876 | 5:100815761 | 26879 | 5:100854950 | 26917 | 5:101625777 | 27085 | 5:105474132 |
| 27094 | 5:105732916 | 27095 | 5:105733857 | 27096 | 5:105740050 | 27097 | 5:105756724 | 27098 | 5:105760018 |
| 27099 | 5:105760750 | 27101 | 5:105770267 | 27102 | 5:105771805 | 27103 | 5:105773809 | 27104 | 5:105776080 |
| 27105 | 5:105776475 | 27106 | 5:105778521 | 27107 | 5:105778777 | 27108 | 5:105779368 | 27109 | 5:105780258 |
| 27110 | 5:105800847 | 27112 | 5:105804880 | 27167 | 5:106540440 | 27175 | 5:106650676 | 27227 | 5:107423031 |
| 27378 | 5:110192579 | 27381 | 5:110289240 | 27391 | 5:110386083 | 27419 | 5:110751512 | 27501 | 5:112519431 |
| 27502 | 5:112553713 | 27533 | 5:113205459 | 27568 | 5:114190671 | 27822 | 5:117930669 | 27862 | 5:118720245 |
| 27891 | 5:119036022 | 27910 | 5:119441240 | 28029 | 6:1993838 | 28117 | 6:3589994 | 28156 | 6:4424160 |
| 28249 | 6:6458237 | 28250 | 6:6482619 | 28394 | 6:10017733 | 28447 | 6:11463553 | 28470 | 6:11875946 |
| 28484 | 6:12191468 | 28503 | 6:12548297 | 28504 | 6:12550413 | 28512 | 6:12753537 | 28513 | 6:12808531 |
| 28515 | 6:12873396 | 28517 | 6:12900652 | 28580 | 6:14515552 | 28582 | 6:14546752 | 28584 | 6:14593717 |
| 28588 | 6:14737560 | 28660 | 6:16069555 | 28665 | 6:16173201 | 28670 | 6:16334539 | 28671 | 6:16387845 |
| 28672 | 6:16407876 | 28700 | 6:16918116 | 28708 | 6:17036127 | 28721 | 6:17373915 | 28727 | 6:17498903 |
| 28786 | 6:18598550 | 28800 | 6:18917943 | 28802 | 6:18955466 | 28828 | 6:19455356 | 28838 | 6:19628808 |
| 28847 | 6:19848821 | 28851 | 6:19923403 | 28858 | 6:20135992 | 28864 | 6:20273699 | 28873 | 6:20399602 |
| 28881 | 6:20600966 | 28884 | 6:20625823 | 28891 | 6:20779771 | 28892 | 6:20821807 | 28906 | 6:21288355 |
| 28909 | 6:21362764 | 28911 | 6:21407401 | 28920 | 6:21614174 | 28924 | 6:21682567 | 28929 | 6:21732184 |
| 28930 | 6:21736509 | 28937 | 6:21857115 | 28943 | 6:22080199 | 28948 | 6:22169669 | 28958 | 6:22265776 |
| 28982 | 6:22743978 | 28986 | 6:22838838 | 29009 | 6:23503327 | 29017 | 6:23665616 | 29019 | 6:23697957 |
| 29025 | 6:23885602 | 29026 | 6:23902363 | 29027 | 6:23902790 | 29037 | 6:24156443 | 29038 | 6:24188452 |
| 29062 | 6:24764063 | 29073 | 6:25006732 | 29074 | 6:25007314 | 29075 | 6:25007705 | 29085 | 6:25188129 |
| 29088 | 6:25333843 | 29095 | 6:25528726 | 29104 | 6:25843075 | 29112 | 6:25978766 | 29117 | 6:26022919 |
| 29125 | 6:26158019 | 29142 | 6:26679658 | 29146 | 6:26760274 | 29148 | 6:26792379 | 29156 | 6:27042457 |
| 29160 | 6:27154761 | 29165 | 6:27332801 | 29171 | 6:27406476 | 29192 | 6:28048764 | 29202 | 6:28285173 |
| 29219 | 6:28873303 | 29221 | 6:28936994 | 29222 | 6:28940832 | 29224 | 6:28978809 | 29229 | 6:29062262 |
| 29242 | 6:29357249 | 29246 | 6:29406846 | 29252 | 6:29478697 | 29261 | 6:29591601 | 29264 | 6:29642427 |
| 29266 | 6:29700842 | 29272 | 6:29850442 | 29275 | 6:29928530 | 29278 | 6:30059483 | 29281 | 6:30087471 |
| 29282 | 6:30092735 | 29283 | 6:30096041 | 29284 | 6:30096126 | 29285 | 6:30100327 | 29286 | 6:30101354 |
| 29288 | 6:30105186 | 29289 | 6:30107161 | 29290 | 6:30107363 | 29291 | 6:30107470 | 29292 | 6:30107503 |
| 29293 | 6:30108091 | 29294 | 6:30108190 | 29295 | 6:30108349 | 29296 | 6:30108502 | 29297 | 6:30109134 |
| 29298 | 6:30109220 | 29299 | 6:30109285 | 29300 | 6:30109297 | 29301 | 6:30109871 | 29302 | 6:30109915 |
| 29303 | 6:30110219 | 29304 | 6:30110252 | 29305 | 6:30110638 | 29306 | 6:30111065 | 29307 | 6:30111645 |
| 29308 | 6:30112226 | 29309 | 6:30112636 | 29310 | 6:30112665 | 29311 | 6:30112688 | 29312 | 6:30112989 |
| 29313 | 6:30113025 | 29314 | 6:30113099 | 29315 | 6:30113113 | 29316 | 6:30113219 | 29317 | 6:30113503 |
| 29318 | 6:30113552 | 29319 | 6:30113737 | 29320 | 6:30114877 | 29321 | 6:30114924 | 29322 | 6:30115248 |
| 29323 | 6:30115405 | 29324 | 6:30115465 | 29325 | 6:30117653 | 29326 | 6:30117880 | 29327 | 6:30117987 |
| 29328 | 6:30118079 | 29329 | 6:30118215 | 29330 | 6:30119075 | 29331 | 6:30119163 | 29332 | 6:30119239 |
| 29333 | 6:30119492 | 29334 | 6:30120062 | 29335 | 6:30120131 | 29336 | 6:30120148 | 29337 | 6:30120317 |
| 29338 | 6:30120495 | 29339 | 6:30120714 | 29340 | 6:30120745 | 29341 | 6:30121010 | 29345 | 6:30186140 |
| 29347 | 6:30231489 | 29348 | 6:30231511 | 29349 | 6:30231899 | 29350 | 6:30231913 | 29351 | 6:30232234 |
| 29356 | 6:30429744 | 29366 | 6:30575474 | 29370 | 6:30706328 | 29373 | 6:30820768 | 29381 | 6:31067757 |
| 29386 | 6:31258805 | 29390 | 6:31380855 | 29392 | 6:31425251 | 29394 | 6:31479415 | 29397 | 6:31502542 |
| 29398 | 6:31523083 | 29409 | 6:31802402 | 29417 | 6:32011040 | 29446 | 6:33106068 | 29447 | 6:33200563 |
| 29448 | 6:33249833 | 29450 | 6:33303040 | 29454 | 6:33451849 | 29456 | 6:33555068 | 29466 | 6:33983856 |
| 29474 | 6:34086214 | 29476 | 6:34139287 | 29477 | 6:34174152 | 29478 | 6:34179094 | 29479 | 6:34185107 |
| 29480 | 6:34189894 | 29481 | 6:34197024 | 29483 | 6:34206187 | 29484 | 6:34207156 | 29485 | 6:34210015 |
| 29487 | 6:34227111 | 29488 | 6:34229172 | 29489 | 6:34236680 | 29491 | 6:34238928 | 29492 | 6:34239830 |
| 29493 | 6:34248001 | 29494 | 6:34262023 | 29495 | 6:34291379 | 29496 | 6:34292850 | 29497 | 6:34293142 |
| 29498 | 6:34297896 | 29499 | 6:34298106 | 29500 | 6:34300136 | 29501 | 6:34305846 | 29502 | 6:34313535 |
| 29503 | 6:34314736 | 29504 | 6:34316483 | 29506 | 6:34317628 | 29507 | 6:34317733 | 29508 | 6:34319685 |
| 29509 | 6:34319748 | 29510 | 6:34319965 | 29511 | 6:34320099 | 29512 | 6:34320208 | 29513 | 6:34320272 |
| 29514 | 6:34320435 | 29515 | 6:34320590 | 29516 | 6:34320597 | 29517 | 6:34321475 | 29518 | 6:34321669 |
| 29519 | 6:34321700 | 29520 | 6:34322765 | 29521 | 6:34323241 | 29522 | 6:34323355 | 29523 | 6:34323382 |
| 29524 | 6:34323399 | 29525 | 6:34323857 | 29526 | 6:34323923 | 29527 | 6:34324379 | 29528 | 6:34324509 |
| 29529 | 6:34324543 | 29530 | 6:34324794 | 29531 | 6:34324813 | 29532 | 6:34324864 | 29533 | 6:34325948 |
| 29534 | 6:34326047 | 29535 | 6:34326105 | 29536 | 6:34326672 | 29537 | 6:34326907 | 29538 | 6:34326931 |
| 29539 | 6:34327343 | 29540 | 6:34328174 | 29541 | 6:34328391 | 29542 | 6:34328595 | 29543 | 6:34328871 |
| 29544 | 6:34329287 | 29545 | 6:34329583 | 29546 | 6:34329816 | 29547 | 6:34329841 | 29548 | 6:34329959 |
| 29549 | 6:34330004 | 29550 | 6:34330105 | 29551 | 6:34330219 | 29552 | 6:34330248 | 29553 | 6:34330469 |
| 29554 | 6:34330615 | 29555 | 6:34331649 | 29556 | 6:34331871 | 29557 | 6:34334654 | 29558 | 6:34334695 |
| 29559 | 6:34334819 | 29560 | 6:34334883 | 29561 | 6:34334942 | 29562 | 6:34334975 | 29563 | 6:34335273 |
| 29564 | 6:34335676 | 29565 | 6:34336082 | 29566 | 6:34336116 | 29567 | 6:34336302 | 29568 | 6:34336518 |
| 29569 | 6:34336806 | 29570 | 6:34337142 | 29571 | 6:34337235 | 29572 | 6:34337793 | 29573 | 6:34337852 |
| 29574 | 6:34337871 | 29575 | 6:34337952 | 29576 | 6:34338244 | 29577 | 6:34338458 | 29578 | 6:34338858 |
| 29579 | 6:34338912 | 29580 | 6:34341641 | 29581 | 6:34341671 | 29582 | 6:34342381 | 29583 | 6:34342418 |
| 29584 | 6:34342439 | 29585 | 6:34342876 | 29586 | 6:34343375 | 29587 | 6:34344002 | 29588 | 6:34344707 |
| 29589 | 6:34344823 | 29590 | 6:34344911 | 29591 | 6:34345770 | 29592 | 6:34345787 | 29593 | 6:34347423 |
| 29594 | 6:34347478 | 29595 | 6:34347542 | 29596 | 6:34347870 | 29597 | 6:34347912 | 29598 | 6:34348158 |
| 29599 | 6:34348200 | 29600 | 6:34350237 | 29601 | 6:34350256 | 29602 | 6:34350284 | 29603 | 6:34352029 |
| 29604 | 6:34352540 | 29605 | 6:34353080 | 29606 | 6:34353086 | 29607 | 6:34353183 | 29608 | 6:34353582 |
| 29609 | 6:34353683 | 29610 | 6:34355309 | 29611 | 6:34356066 | 29612 | 6:34356817 | 29613 | 6:34357441 |
| 29614 | 6:34357724 | 29615 | 6:34358059 | 29616 | 6:34358159 | 29617 | 6:34359885 | 29618 | 6:34359922 |
| 29619 | 6:34360320 | 29620 | 6:34360574 | 29621 | 6:34360625 | 29622 | 6:34361019 | 29623 | 6:34361442 |
| 29624 | 6:34362625 | 29625 | 6:34363701 | 29626 | 6:34366860 | 29627 | 6:34367704 | 29628 | 6:34368646 |
| 29629 | 6:34368728 | 29630 | 6:34368895 | 29631 | 6:34369801 | 29632 | 6:34370381 | 29633 | 6:34373419 |
| 29634 | 6:34375386 | 29635 | 6:34377523 | 29636 | 6:34378377 | 29637 | 6:34378764 | 29638 | 6:34379342 |
| 29639 | 6:34379765 | 29640 | 6:34380266 | 29641 | 6:34380420 | 29642 | 6:34390859 | 29643 | 6:34398273 |
| 29644 | 6:34400215 | 29645 | 6:34401918 | 29646 | 6:34405865 | 29647 | 6:34409323 | 29648 | 6:34412354 |
| 29649 | 6:34412446 | 29650 | 6:34417593 | 29651 | 6:34423276 | 29652 | 6:34425931 | 29653 | 6:34429298 |
| 29654 | 6:34433083 | 29655 | 6:34436717 | 29656 | 6:34443045 | 29657 | 6:34445489 | 29658 | 6:34454602 |
| 29661 | 6:34485191 | 29663 | 6:34508971 | 29664 | 6:34511326 | 29665 | 6:34512053 | 29666 | 6:34512149 |
| 29667 | 6:34512224 | 29668 | 6:34512436 | 29669 | 6:34512607 | 29670 | 6:34514009 | 29671 | 6:34520073 |
| 29672 | 6:34520205 | 29673 | 6:34520340 | 29674 | 6:34521287 | 29675 | 6:34521294 | 29676 | 6:34522877 |
| 29677 | 6:34523485 | 29678 | 6:34524720 | 29679 | 6:34526737 | 29680 | 6:34527170 | 29681 | 6:34531081 |
| 29682 | 6:34531219 | 29683 | 6:34531228 | 29684 | 6:34532115 | 29685 | 6:34532276 | 29686 | 6:34533970 |
| 29687 | 6:34534131 | 29688 | 6:34534507 | 29689 | 6:34534849 | 29690 | 6:34534873 | 29691 | 6:34535922 |
| 29692 | 6:34535958 | 29693 | 6:34535980 | 29694 | 6:34537513 | 29695 | 6:34538518 | 29696 | 6:34538586 |
| 29697 | 6:34538798 | 29698 | 6:34539189 | 29699 | 6:34544253 | 29700 | 6:34544296 | 29701 | 6:34544650 |
| 29702 | 6:34544882 | 29703 | 6:34544924 | 29704 | 6:34545143 | 29705 | 6:34545442 | 29706 | 6:34545976 |
| 29707 | 6:34546002 | 29708 | 6:34546098 | 29709 | 6:34546230 | 29710 | 6:34546882 | 29711 | 6:34551061 |
| 29712 | 6:34551087 | 29713 | 6:34551357 | 29714 | 6:34551821 | 29715 | 6:34552091 | 29716 | 6:34552302 |
| 29717 | 6:34559140 | 29719 | 6:34568139 | 29720 | 6:34570377 | 29721 | 6:34571403 | 29723 | 6:34585218 |
| 29724 | 6:34587383 | 29726 | 6:34590054 | 29727 | 6:34590568 | 29728 | 6:34590836 | 29729 | 6:34590891 |
| 29730 | 6:34594553 | 29731 | 6:34595913 | 29732 | 6:34605484 | 29734 | 6:34606444 | 29735 | 6:34606508 |
| 29736 | 6:34607125 | 29737 | 6:34607239 | 29738 | 6:34608122 | 29739 | 6:34608604 | 29740 | 6:34609191 |
| 29741 | 6:34609820 | 29742 | 6:34609854 | 29743 | 6:34610451 | 29744 | 6:34610729 | 29745 | 6:34610740 |
| 29746 | 6:34610765 | 29748 | 6:34610849 | 29749 | 6:34611386 | 29750 | 6:34611923 | 29751 | 6:34611955 |
| 29752 | 6:34612600 | 29753 | 6:34613447 | 29754 | 6:34614887 | 29755 | 6:34615063 | 29756 | 6:34617013 |
| 29757 | 6:34617195 | 29758 | 6:34617208 | 29759 | 6:34617293 | 29760 | 6:34617674 | 29761 | 6:34617812 |
| 29762 | 6:34617865 | 29763 | 6:34617943 | 29764 | 6:34618113 | 29765 | 6:34618226 | 29766 | 6:34618258 |
| 29767 | 6:34618461 | 29768 | 6:34618467 | 29769 | 6:34618532 | 29770 | 6:34618622 | 29771 | 6:34618679 |
| 29772 | 6:34618784 | 29773 | 6:34618813 | 29774 | 6:34618880 | 29775 | 6:34620004 | 29776 | 6:34620108 |
| 29777 | 6:34620118 | 29778 | 6:34620548 | 29779 | 6:34621825 | 29780 | 6:34622374 | 29781 | 6:34623163 |
| 29782 | 6:34623346 | 29783 | 6:34623681 | 29784 | 6:34623706 | 29785 | 6:34623745 | 29786 | 6:34624441 |
| 29787 | 6:34629181 | 29788 | 6:34629567 | 29789 | 6:34629581 | 29790 | 6:34629692 | 29791 | 6:34629900 |
| 29792 | 6:34629975 | 29793 | 6:34629990 | 29794 | 6:34630073 | 29795 | 6:34630347 | 29796 | 6:34630587 |
| 29797 | 6:34630602 | 29798 | 6:34630922 | 29799 | 6:34631246 | 29800 | 6:34631446 | 29801 | 6:34631808 |
| 29802 | 6:34631843 | 29803 | 6:34632098 | 29804 | 6:34632375 | 29805 | 6:34632405 | 29806 | 6:34632714 |
| 29807 | 6:34632833 | 29808 | 6:34633050 | 29809 | 6:34633159 | 29810 | 6:34633187 | 29811 | 6:34633229 |
| 29812 | 6:34633562 | 29813 | 6:34633782 | 29814 | 6:34633841 | 29815 | 6:34633967 | 29816 | 6:34633990 |
| 29817 | 6:34634055 | 29818 | 6:34634169 | 29819 | 6:34634190 | 29820 | 6:34634250 | 29821 | 6:34634312 |
| 29822 | 6:34634801 | 29823 | 6:34634990 | 29824 | 6:34635003 | 29825 | 6:34635530 | 29826 | 6:34635703 |
| 29827 | 6:34636131 | 29828 | 6:34636138 | 29829 | 6:34636209 | 29830 | 6:34637085 | 29831 | 6:34640553 |
| 29832 | 6:34641695 | 29833 | 6:34647407 | 29834 | 6:34657657 | 29835 | 6:34661296 | 29836 | 6:34661323 |
| 29837 | 6:34662849 | 29838 | 6:34665463 | 29839 | 6:34668000 | 29840 | 6:34670574 | 29841 | 6:34671333 |
| 29842 | 6:34673238 | 29844 | 6:34686618 | 29845 | 6:34687343 | 29846 | 6:34689725 | 29847 | 6:34697260 |
| 29848 | 6:34699079 | 29849 | 6:34703681 | 29850 | 6:34704601 | 29851 | 6:34704806 | 29852 | 6:34705455 |
| 29853 | 6:34714302 | 29854 | 6:34714956 | 29855 | 6:34724151 | 29856 | 6:34726853 | 29857 | 6:34727995 |
| 29858 | 6:34728449 | 29859 | 6:34730148 | 29860 | 6:34730862 | 29861 | 6:34734530 | 29863 | 6:34738528 |
| 29864 | 6:34746517 | 29865 | 6:34751152 | 29866 | 6:34751196 | 29867 | 6:34751889 | 29868 | 6:34753115 |
| 29869 | 6:34762474 | 29870 | 6:34762792 | 29871 | 6:34762888 | 29872 | 6:34766154 | 29873 | 6:34766590 |
| 29874 | 6:34766756 | 29875 | 6:34768280 | 29876 | 6:34768301 | 29877 | 6:34768997 | 29878 | 6:34769036 |
| 29879 | 6:34769720 | 29880 | 6:34770109 | 29881 | 6:34770808 | 29882 | 6:34777728 | 29883 | 6:34780553 |
| 29884 | 6:34781550 | 29885 | 6:34781934 | 29886 | 6:34782739 | 29887 | 6:34782759 | 29888 | 6:34783042 |
| 29889 | 6:34783961 | 29890 | 6:34784243 | 29891 | 6:34784299 | 29892 | 6:34784335 | 29893 | 6:34784977 |
| 29894 | 6:34785811 | 29895 | 6:34785864 | 29896 | 6:34785916 | 29897 | 6:34786001 | 29898 | 6:34786006 |
| 29899 | 6:34786198 | 29900 | 6:34786203 | 29901 | 6:34786474 | 29902 | 6:34786586 | 29903 | 6:34787240 |
| 29904 | 6:34789109 | 29905 | 6:34792867 | 29907 | 6:34845861 | 29909 | 6:34929550 | 29910 | 6:34932019 |
| 29911 | 6:34933373 | 29912 | 6:34943666 | 29913 | 6:34961634 | 29915 | 6:34972640 | 29916 | 6:34976990 |
| 29917 | 6:34985119 | 29918 | 6:34990679 | 29919 | 6:34992674 | 29920 | 6:34994097 | 29921 | 6:34995220 |
| 29922 | 6:35021191 | 29923 | 6:35047210 | 29924 | 6:35140608 | 29927 | 6:35157410 | 29929 | 6:35160353 |
| 29930 | 6:35161520 | 29932 | 6:35162419 | 29934 | 6:35163337 | 29935 | 6:35165014 | 29936 | 6:35166488 |
| 29937 | 6:35166725 | 29942 | 6:35245781 | 29945 | 6:35274129 | 29948 | 6:35381936 | 29953 | 6:35504728 |
| 29957 | 6:35561016 | 29958 | 6:35561546 | 29959 | 6:35561595 | 29960 | 6:35581628 | 29961 | 6:35582162 |
| 29962 | 6:35582223 | 29963 | 6:35587094 | 29964 | 6:35594486 | 29965 | 6:35609063 | 29966 | 6:35616265 |
| 29967 | 6:35616280 | 29969 | 6:35618707 | 29974 | 6:35666246 | 29975 | 6:35678704 | 29976 | 6:35678754 |
| 29977 | 6:35684254 | 29978 | 6:35703001 | 29979 | 6:35713846 | 29980 | 6:35718341 | 29981 | 6:35726574 |
| 29982 | 6:35732398 | 29984 | 6:35792391 | 29985 | 6:35825879 | 29987 | 6:35886281 | 29989 | 6:35897314 |
| 29994 | 6:35908980 | 30001 | 6:35937425 | 30002 | 6:35937819 | 30003 | 6:35937874 | 30004 | 6:35941557 |
| 30006 | 6:35948457 | 30007 | 6:35963118 | 30008 | 6:35968631 | 30009 | 6:35972831 | 30012 | 6:35992028 |
| 30014 | 6:36011027 | 30015 | 6:36011772 | 30018 | 6:36032905 | 30019 | 6:36034678 | 30020 | 6:36036595 |
| 30029 | 6:36125455 | 30030 | 6:36143409 | 30031 | 6:36145256 | 30032 | 6:36147418 | 30033 | 6:36148375 |
| 30034 | 6:36149041 | 30035 | 6:36150876 | 30036 | 6:36151325 | 30037 | 6:36153013 | 30038 | 6:36156606 |
| 30039 | 6:36156894 | 30040 | 6:36157622 | 30041 | 6:36157988 | 30042 | 6:36158361 | 30043 | 6:36158500 |
| 30044 | 6:36159608 | 30045 | 6:36161275 | 30046 | 6:36161377 | 30047 | 6:36161627 | 30048 | 6:36161635 |
| 30049 | 6:36162952 | 30050 | 6:36163342 | 30051 | 6:36163430 | 30052 | 6:36164022 | 30053 | 6:36164566 |
| 30054 | 6:36165003 | 30055 | 6:36165139 | 30056 | 6:36165152 | 30057 | 6:36165220 | 30058 | 6:36165307 |
| 30059 | 6:36166110 | 30060 | 6:36166180 | 30061 | 6:36166207 | 30062 | 6:36166505 | 30063 | 6:36166793 |
| 30064 | 6:36166797 | 30065 | 6:36167327 | 30066 | 6:36167830 | 30067 | 6:36168126 | 30068 | 6:36168722 |
| 30069 | 6:36169714 | 30070 | 6:36169794 | 30071 | 6:36170135 | 30072 | 6:36170201 | 30073 | 6:36171686 |
| 30074 | 6:36172372 | 30075 | 6:36177201 | 30076 | 6:36180645 | 30077 | 6:36181021 | 30078 | 6:36182794 |
| 30079 | 6:36183913 | 30080 | 6:36186583 | 30081 | 6:36191256 | 30082 | 6:36198702 | 30083 | 6:36204036 |
| 30084 | 6:36211597 | 30085 | 6:36212186 | 30086 | 6:36219844 | 30087 | 6:36225618 | 30088 | 6:36236546 |
| 30089 | 6:36238601 | 30090 | 6:36238793 | 30091 | 6:36239080 | 30092 | 6:36239773 | 30093 | 6:36240264 |
| 30094 | 6:36241246 | 30095 | 6:36246325 | 30096 | 6:36248262 | 30097 | 6:36249143 | 30098 | 6:36250084 |
| 30099 | 6:36254264 | 30100 | 6:36255334 | 30101 | 6:36256227 | 30102 | 6:36256264 | 30103 | 6:36256316 |
| 30104 | 6:36261278 | 30105 | 6:36262429 | 30106 | 6:36263719 | 30107 | 6:36264671 | 30108 | 6:36264792 |
| 30109 | 6:36265379 | 30110 | 6:36272248 | 30111 | 6:36273670 | 30112 | 6:36277826 | 30113 | 6:36280253 |
| 30114 | 6:36283110 | 30115 | 6:36283198 | 30116 | 6:36286200 | 30117 | 6:36292811 | 30118 | 6:36298957 |
| 30119 | 6:36299463 | 30120 | 6:36299867 | 30121 | 6:36300007 | 30122 | 6:36324361 | 30123 | 6:36333247 |
| 30124 | 6:36336622 | 30125 | 6:36336765 | 30126 | 6:36337366 | 30127 | 6:36340265 | 30128 | 6:36363919 |
| 30129 | 6:36365275 | 30131 | 6:36412092 | 30141 | 6:36436269 | 30142 | 6:36440409 | 30143 | 6:36441375 |
| 30144 | 6:36443819 | 30146 | 6:36445226 | 30149 | 6:36454421 | 30151 | 6:36457471 | 30152 | 6:36457509 |
| 30153 | 6:36458519 | 30154 | 6:36460784 | 30155 | 6:36461234 | 30156 | 6:36461574 | 30157 | 6:36463931 |
| 30158 | 6:36466353 | 30159 | 6:36466871 | 30161 | 6:36469381 | 30162 | 6:36469975 | 30163 | 6:36474704 |
| 30166 | 6:36478628 | 30167 | 6:36480305 | 30170 | 6:36482113 | 30172 | 6:36487754 | 30173 | 6:36489160 |
| 30176 | 6:36491203 | 30177 | 6:36491990 | 30178 | 6:36499104 | 30179 | 6:36499659 | 30180 | 6:36500389 |
| 30181 | 6:36501474 | 30182 | 6:36503509 | 30183 | 6:36506439 | 30184 | 6:36506901 | 30185 | 6:36509073 |
| 30186 | 6:36514666 | 30187 | 6:36514687 | 30188 | 6:36516071 | 30189 | 6:36518117 | 30190 | 6:36524445 |
| 30191 | 6:36524489 | 30192 | 6:36529972 | 30193 | 6:36530565 | 30194 | 6:36531251 | 30195 | 6:36531630 |
| 30197 | 6:36532751 | 30198 | 6:36533529 | 30199 | 6:36533756 | 30200 | 6:36533947 | 30201 | 6:36534891 |
| 30202 | 6:36535012 | 30203 | 6:36535227 | 30204 | 6:36535356 | 30205 | 6:36535863 | 30206 | 6:36535904 |
| 30207 | 6:36536565 | 30208 | 6:36537055 | 30209 | 6:36537210 | 30210 | 6:36537223 | 30211 | 6:36537284 |
| 30212 | 6:36537515 | 30213 | 6:36537657 | 30214 | 6:36537708 | 30215 | 6:36538044 | 30216 | 6:36538107 |
| 30217 | 6:36538113 | 30218 | 6:36538805 | 30219 | 6:36538827 | 30220 | 6:36539051 | 30221 | 6:36539508 |
| 30222 | 6:36539630 | 30223 | 6:36540193 | 30224 | 6:36540734 | 30225 | 6:36540815 | 30226 | 6:36543846 |
| 30227 | 6:36544549 | 30228 | 6:36544681 | 30229 | 6:36544687 | 30230 | 6:36544786 | 30231 | 6:36544914 |
| 30232 | 6:36545202 | 30233 | 6:36545304 | 30235 | 6:36547031 | 30236 | 6:36547078 | 30237 | 6:36547888 |
| 30238 | 6:36547972 | 30239 | 6:36548012 | 30240 | 6:36548205 | 30241 | 6:36548953 | 30242 | 6:36549261 |
| 30243 | 6:36549683 | 30244 | 6:36550069 | 30245 | 6:36550199 | 30246 | 6:36551621 | 30247 | 6:36552037 |
| 30248 | 6:36552742 | 30249 | 6:36552915 | 30252 | 6:36556271 | 30253 | 6:36556442 | 30254 | 6:36557671 |
| 30256 | 6:36561952 | 30258 | 6:36567616 | 30262 | 6:36569777 | 30263 | 6:36571430 | 30265 | 6:36594692 |
| 30266 | 6:36598347 | 30267 | 6:36599192 | 30268 | 6:36602971 | 30269 | 6:36603958 | 30270 | 6:36608521 |
| 30271 | 6:36608642 | 30272 | 6:36611873 | 30273 | 6:36612067 | 30274 | 6:36614641 | 30275 | 6:36617150 |
| 30276 | 6:36618712 | 30277 | 6:36619708 | 30278 | 6:36620654 | 30279 | 6:36621895 | 30280 | 6:36622608 |
| 30281 | 6:36624334 | 30282 | 6:36625203 | 30283 | 6:36625485 | 30285 | 6:36626119 | 30286 | 6:36626509 |
| 30287 | 6:36629387 | 30288 | 6:36631261 | 30289 | 6:36632276 | 30290 | 6:36632779 | 30291 | 6:36633630 |
| 30292 | 6:36633929 | 30293 | 6:36634989 | 30294 | 6:36635404 | 30295 | 6:36635605 | 30296 | 6:36635939 |
| 30297 | 6:36638521 | 30298 | 6:36638963 | 30299 | 6:36639106 | 30300 | 6:36642083 | 30301 | 6:36642499 |
| 30302 | 6:36643100 | 30303 | 6:36643378 | 30304 | 6:36644151 | 30305 | 6:36645330 | 30306 | 6:36646194 |
| 30307 | 6:36646596 | 30308 | 6:36648068 | 30309 | 6:36648160 | 30310 | 6:36653563 | 30311 | 6:36657483 |
| 30312 | 6:36664663 | 30313 | 6:36673216 | 30314 | 6:36679692 | 30316 | 6:36681772 | 30318 | 6:36695293 |
| 30319 | 6:36700131 | 30320 | 6:36700135 | 30321 | 6:36706370 | 30322 | 6:36720331 | 30324 | 6:36743854 |
| 30325 | 6:36773960 | 30326 | 6:36787210 | 30331 | 6:36812951 | 30332 | 6:36813102 | 30333 | 6:36813124 |
| 30334 | 6:36813128 | 30335 | 6:36813593 | 30336 | 6:36813633 | 30337 | 6:36814581 | 30338 | 6:36814786 |
| 30339 | 6:36815144 | 30340 | 6:36815200 | 30341 | 6:36815490 | 30342 | 6:36815725 | 30343 | 6:36815817 |
| 30344 | 6:36816067 | 30345 | 6:36816314 | 30346 | 6:36816419 | 30347 | 6:36817159 | 30348 | 6:36817266 |
| 30349 | 6:36817581 | 30350 | 6:36817596 | 30351 | 6:36817683 | 30352 | 6:36817713 | 30353 | 6:36817969 |
| 30354 | 6:36818237 | 30355 | 6:36818704 | 30356 | 6:36818808 | 30357 | 6:36819594 | 30358 | 6:36819695 |
| 30359 | 6:36819751 | 30360 | 6:36820074 | 30361 | 6:36820138 | 30362 | 6:36820361 | 30363 | 6:36820770 |
| 30364 | 6:36821432 | 30365 | 6:36821474 | 30366 | 6:36822273 | 30367 | 6:36822526 | 30368 | 6:36822568 |
| 30369 | 6:36822744 | 30370 | 6:36823102 | 30371 | 6:36823606 | 30372 | 6:36825509 | 30373 | 6:36830103 |
| 30374 | 6:36830934 | 30375 | 6:36831012 | 30376 | 6:36831131 | 30377 | 6:36831274 | 30378 | 6:36831813 |
| 30379 | 6:36831871 | 30380 | 6:36834692 | 30381 | 6:36835108 | 30391 | 6:36949956 | 30392 | 6:36952579 |
| 30393 | 6:36954127 | 30395 | 6:36959305 | 30396 | 6:36960146 | 30397 | 6:36970451 | 30398 | 6:36978207 |
| 30399 | 6:36978216 | 30400 | 6:36978361 | 30401 | 6:36978371 | 30402 | 6:36978422 | 30403 | 6:36978431 |
| 30404 | 6:36978698 | 30405 | 6:36978703 | 30406 | 6:36978958 | 30407 | 6:36979025 | 30408 | 6:36979081 |
| 30409 | 6:36979093 | 30410 | 6:36979116 | 30411 | 6:36979134 | 30412 | 6:36979146 | 30413 | 6:36981664 |
| 30414 | 6:36981815 | 30415 | 6:36985783 | 30416 | 6:36985804 | 30417 | 6:36995904 | 30419 | 6:37010986 |
| 30420 | 6:37011036 | 30421 | 6:37016748 | 30422 | 6:37017628 | 30423 | 6:37017833 | 30424 | 6:37018373 |
| 30425 | 6:37018495 | 30426 | 6:37018672 | 30427 | 6:37018745 | 30428 | 6:37018878 | 30429 | 6:37019035 |
| 30430 | 6:37019263 | 30431 | 6:37019274 | 30432 | 6:37019373 | 30433 | 6:37019415 | 30434 | 6:37019464 |
| 30435 | 6:37019512 | 30436 | 6:37019519 | 30437 | 6:37019606 | 30438 | 6:37020100 | 30439 | 6:37020244 |
| 30440 | 6:37020255 | 30441 | 6:37020456 | 30442 | 6:37020574 | 30443 | 6:37020591 | 30444 | 6:37020608 |
| 30445 | 6:37021123 | 30446 | 6:37021154 | 30447 | 6:37021520 | 30448 | 6:37021604 | 30449 | 6:37021714 |
| 30450 | 6:37021968 | 30451 | 6:37022137 | 30452 | 6:37022181 | 30453 | 6:37022238 | 30454 | 6:37023645 |
| 30460 | 6:37030480 | 30461 | 6:37032906 | 30463 | 6:37034096 | 30464 | 6:37034286 | 30465 | 6:37034823 |
| 30466 | 6:37034912 | 30467 | 6:37035186 | 30469 | 6:37038017 | 30470 | 6:37044068 | 30473 | 6:37061527 |
| 30474 | 6:37074563 | 30475 | 6:37075910 | 30476 | 6:37078470 | 30477 | 6:37078665 | 30478 | 6:37078781 |
| 30479 | 6:37079090 | 30480 | 6:37079147 | 30481 | 6:37079324 | 30482 | 6:37079422 | 30483 | 6:37079559 |
| 30484 | 6:37079653 | 30485 | 6:37079666 | 30486 | 6:37080062 | 30487 | 6:37080097 | 30488 | 6:37080210 |
| 30489 | 6:37080486 | 30490 | 6:37080919 | 30491 | 6:37080936 | 30492 | 6:37081034 | 30493 | 6:37081117 |
| 30494 | 6:37081212 | 30495 | 6:37081319 | 30496 | 6:37081745 | 30497 | 6:37081947 | 30498 | 6:37082116 |
| 30499 | 6:37082537 | 30500 | 6:37082542 | 30501 | 6:37082592 | 30502 | 6:37082641 | 30503 | 6:37082764 |
| 30504 | 6:37082830 | 30505 | 6:37083023 | 30506 | 6:37083086 | 30507 | 6:37083196 | 30508 | 6:37083881 |
| 30509 | 6:37084518 | 30510 | 6:37084543 | 30511 | 6:37084979 | 30512 | 6:37084987 | 30513 | 6:37084991 |
| 30514 | 6:37085423 | 30515 | 6:37085516 | 30516 | 6:37087274 | 30517 | 6:37087642 | 30518 | 6:37087987 |
| 30519 | 6:37087999 | 30520 | 6:37088359 | 30521 | 6:37088506 | 30522 | 6:37088510 | 30523 | 6:37088831 |
| 30524 | 6:37088835 | 30525 | 6:37088955 | 30526 | 6:37089045 | 30527 | 6:37089108 | 30528 | 6:37089125 |
| 30529 | 6:37089202 | 30530 | 6:37089209 | 30531 | 6:37089234 | 30532 | 6:37089651 | 30533 | 6:37089673 |
| 30534 | 6:37089763 | 30535 | 6:37089819 | 30536 | 6:37089854 | 30537 | 6:37089926 | 30538 | 6:37090001 |
| 30539 | 6:37090127 | 30540 | 6:37090147 | 30541 | 6:37090190 | 30542 | 6:37090229 | 30543 | 6:37090257 |
| 30544 | 6:37090343 | 30545 | 6:37090838 | 30546 | 6:37090865 | 30547 | 6:37091359 | 30548 | 6:37091365 |
| 30549 | 6:37091616 | 30550 | 6:37091767 | 30551 | 6:37092094 | 30552 | 6:37092116 | 30553 | 6:37092227 |
| 30554 | 6:37092573 | 30555 | 6:37092869 | 30556 | 6:37092904 | 30557 | 6:37092936 | 30558 | 6:37093073 |
| 30559 | 6:37093934 | 30560 | 6:37093949 | 30561 | 6:37094212 | 30562 | 6:37094603 | 30563 | 6:37094693 |
| 30564 | 6:37094789 | 30565 | 6:37094827 | 30566 | 6:37095299 | 30567 | 6:37095517 | 30568 | 6:37096027 |
| 30569 | 6:37096074 | 30570 | 6:37096486 | 30571 | 6:37096495 | 30572 | 6:37096649 | 30573 | 6:37096879 |
| 30574 | 6:37096959 | 30575 | 6:37097509 | 30576 | 6:37097522 | 30577 | 6:37097635 | 30578 | 6:37097772 |
| 30579 | 6:37097995 | 30580 | 6:37098052 | 30581 | 6:37098220 | 30582 | 6:37098309 | 30583 | 6:37098640 |
| 30584 | 6:37098716 | 30585 | 6:37098782 | 30586 | 6:37098831 | 30587 | 6:37098872 | 30588 | 6:37099284 |
| 30589 | 6:37099535 | 30590 | 6:37099560 | 30591 | 6:37099942 | 30592 | 6:37099978 | 30593 | 6:37099983 |
| 30594 | 6:37100199 | 30595 | 6:37100203 | 30596 | 6:37100223 | 30597 | 6:37100242 | 30598 | 6:37100749 |
| 30599 | 6:37100852 | 30600 | 6:37100906 | 30601 | 6:37100924 | 30602 | 6:37101114 | 30603 | 6:37101213 |
| 30604 | 6:37101414 | 30605 | 6:37101664 | 30606 | 6:37101691 | 30607 | 6:37104751 | 30608 | 6:37104774 |
| 30609 | 6:37104851 | 30610 | 6:37104926 | 30611 | 6:37104993 | 30612 | 6:37105044 | 30613 | 6:37105252 |
| 30614 | 6:37105257 | 30615 | 6:37106212 | 30616 | 6:37107589 | 30617 | 6:37107774 | 30618 | 6:37108028 |
| 30619 | 6:37108237 | 30620 | 6:37112827 | 30621 | 6:37112864 | 30622 | 6:37116160 | 30623 | 6:37116221 |
| 30624 | 6:37116481 | 30625 | 6:37116730 | 30626 | 6:37117771 | 30627 | 6:37117988 | 30628 | 6:37120342 |
| 30630 | 6:37123023 | 30631 | 6:37128820 | 30634 | 6:37136040 | 30635 | 6:37136706 | 30636 | 6:37136794 |
| 30637 | 6:37138117 | 30638 | 6:37138139 | 30639 | 6:37138262 | 30640 | 6:37138535 | 30641 | 6:37139425 |
| 30642 | 6:37140287 | 30643 | 6:37142174 | 30644 | 6:37142953 | 30645 | 6:37144122 | 30646 | 6:37147544 |
| 30647 | 6:37147627 | 30648 | 6:37149236 | 30649 | 6:37149945 | 30650 | 6:37150579 | 30651 | 6:37151586 |
| 30652 | 6:37151618 | 30654 | 6:37152332 | 30656 | 6:37153197 | 30657 | 6:37153310 | 30658 | 6:37153398 |
| 30659 | 6:37153558 | 30660 | 6:37153831 | 30661 | 6:37155597 | 30662 | 6:37156064 | 30663 | 6:37156337 |
| 30664 | 6:37156677 | 30665 | 6:37156889 | 30666 | 6:37157333 | 30667 | 6:37157660 | 30668 | 6:37158136 |
| 30669 | 6:37158289 | 30670 | 6:37158384 | 30671 | 6:37159298 | 30672 | 6:37159341 | 30673 | 6:37159410 |
| 30674 | 6:37159427 | 30675 | 6:37159591 | 30676 | 6:37159718 | 30677 | 6:37159778 | 30678 | 6:37159892 |
| 30679 | 6:37160005 | 30680 | 6:37160134 | 30681 | 6:37160139 | 30682 | 6:37160559 | 30683 | 6:37161039 |
| 30684 | 6:37161146 | 30685 | 6:37161408 | 30686 | 6:37161619 | 30687 | 6:37161712 | 30688 | 6:37162703 |
| 30689 | 6:37162813 | 30690 | 6:37162822 | 30691 | 6:37163101 | 30692 | 6:37163223 | 30693 | 6:37163776 |
| 30694 | 6:37163934 | 30695 | 6:37164094 | 30696 | 6:37164218 | 30697 | 6:37164439 | 30698 | 6:37164529 |
| 30699 | 6:37164825 | 30700 | 6:37165013 | 30701 | 6:37165306 | 30702 | 6:37165536 | 30703 | 6:37165545 |
| 30704 | 6:37165588 | 30705 | 6:37165831 | 30706 | 6:37165836 | 30707 | 6:37166157 | 30708 | 6:37166449 |
| 30709 | 6:37166482 | 30710 | 6:37167166 | 30711 | 6:37167349 | 30712 | 6:37167472 | 30713 | 6:37167487 |
| 30714 | 6:37167778 | 30715 | 6:37167868 | 30716 | 6:37167910 | 30717 | 6:37167946 | 30718 | 6:37168208 |
| 30719 | 6:37168379 | 30720 | 6:37168414 | 30721 | 6:37168500 | 30722 | 6:37168914 | 30723 | 6:37168959 |
| 30724 | 6:37169024 | 30725 | 6:37169066 | 30726 | 6:37169247 | 30727 | 6:37169728 | 30728 | 6:37169746 |
| 30729 | 6:37169824 | 30730 | 6:37171398 | 30731 | 6:37171831 | 30732 | 6:37172063 | 30733 | 6:37172194 |
| 30734 | 6:37172300 | 30736 | 6:37173048 | 30737 | 6:37173169 | 30738 | 6:37173382 | 30739 | 6:37174433 |
| 30740 | 6:37174739 | 30741 | 6:37174812 | 30742 | 6:37174838 | 30743 | 6:37174847 | 30744 | 6:37174944 |
| 30745 | 6:37175216 | 30746 | 6:37175405 | 30747 | 6:37175422 | 30749 | 6:37175709 | 30751 | 6:37175915 |
| 30752 | 6:37175930 | 30753 | 6:37175957 | 30754 | 6:37176060 | 30755 | 6:37176229 | 30756 | 6:37176528 |
| 30758 | 6:37176686 | 30759 | 6:37177441 | 30760 | 6:37177466 | 30761 | 6:37177638 | 30762 | 6:37177788 |
| 30763 | 6:37177792 | 30764 | 6:37177809 | 30765 | 6:37178122 | 30766 | 6:37178336 | 30767 | 6:37178722 |
| 30768 | 6:37179535 | 30769 | 6:37180932 | 30770 | 6:37180966 | 30771 | 6:37181059 | 30772 | 6:37181087 |
| 30773 | 6:37181206 | 30774 | 6:37181374 | 30775 | 6:37181533 | 30776 | 6:37181894 | 30777 | 6:37182068 |
| 30778 | 6:37182373 | 30779 | 6:37182427 | 30780 | 6:37182621 | 30782 | 6:37182810 | 30783 | 6:37182983 |
| 30784 | 6:37183090 | 30785 | 6:37183609 | 30786 | 6:37183649 | 30789 | 6:37185316 | 30790 | 6:37188198 |
| 30793 | 6:37191616 | 30795 | 6:37193241 | 30796 | 6:37195555 | 30800 | 6:37211057 | 30801 | 6:37214068 |
| 30802 | 6:37214389 | 30803 | 6:37214736 | 30804 | 6:37216084 | 30805 | 6:37216293 | 30806 | 6:37217600 |
| 30807 | 6:37218354 | 30808 | 6:37218423 | 30809 | 6:37219372 | 30811 | 6:37221337 | 30812 | 6:37221507 |
| 30813 | 6:37221776 | 30814 | 6:37222424 | 30815 | 6:37225729 | 30816 | 6:37226016 | 30817 | 6:37226725 |
| 30819 | 6:37228661 | 30821 | 6:37231769 | 30822 | 6:37232090 | 30823 | 6:37232142 | 30824 | 6:37232577 |
| 30825 | 6:37233429 | 30826 | 6:37233659 | 30827 | 6:37234042 | 30828 | 6:37234954 | 30831 | 6:37236226 |
| 30832 | 6:37237969 | 30833 | 6:37238128 | 30834 | 6:37238402 | 30835 | 6:37238486 | 30836 | 6:37238502 |
| 30837 | 6:37238713 | 30838 | 6:37238946 | 30839 | 6:37241792 | 30840 | 6:37241812 | 30841 | 6:37243159 |
| 30842 | 6:37243373 | 30843 | 6:37243677 | 30845 | 6:37244804 | 30846 | 6:37245026 | 30847 | 6:37245138 |
| 30849 | 6:37247874 | 30850 | 6:37248754 | 30853 | 6:37251182 | 30854 | 6:37252978 | 30855 | 6:37255945 |
| 30860 | 6:37263319 | 30861 | 6:37264863 | 30862 | 6:37265148 | 30863 | 6:37266735 | 30866 | 6:37270930 |
| 30868 | 6:37273774 | 30869 | 6:37278799 | 30870 | 6:37281308 | 30871 | 6:37281335 | 30872 | 6:37282356 |
| 30873 | 6:37284874 | 30874 | 6:37285174 | 30875 | 6:37285822 | 30876 | 6:37285843 | 30877 | 6:37286325 |
| 30878 | 6:37286380 | 30880 | 6:37286722 | 30889 | 6:37289572 | 30904 | 6:37293699 | 30906 | 6:37294860 |
| 30907 | 6:37295049 | 30908 | 6:37295154 | 30909 | 6:37295245 | 30910 | 6:37295403 | 30911 | 6:37295803 |
| 30912 | 6:37296114 | 30913 | 6:37297426 | 30914 | 6:37297697 | 30915 | 6:37298251 | 30916 | 6:37298617 |
| 30917 | 6:37298725 | 30918 | 6:37298786 | 30919 | 6:37299349 | 30920 | 6:37299587 | 30921 | 6:37299855 |
| 30922 | 6:37299968 | 30923 | 6:37300447 | 30924 | 6:37300499 | 30925 | 6:37300590 | 30926 | 6:37300767 |
| 30927 | 6:37300810 | 30928 | 6:37300872 | 30929 | 6:37301720 | 30930 | 6:37301762 | 30931 | 6:37301893 |
| 30932 | 6:37301959 | 30933 | 6:37302011 | 30934 | 6:37302172 | 30935 | 6:37302478 | 30936 | 6:37302499 |
| 30937 | 6:37302730 | 30938 | 6:37303255 | 30939 | 6:37303264 | 30940 | 6:37303500 | 30941 | 6:37303542 |
| 30942 | 6:37303727 | 30943 | 6:37304109 | 30944 | 6:37304324 | 30945 | 6:37304493 | 30947 | 6:37304564 |
| 30948 | 6:37304601 | 30949 | 6:37304640 | 30950 | 6:37304825 | 30951 | 6:37305062 | 30952 | 6:37305466 |
| 30953 | 6:37305470 | 30954 | 6:37305490 | 30955 | 6:37305557 | 30956 | 6:37306944 | 30957 | 6:37306975 |
| 30958 | 6:37307238 | 30961 | 6:37310693 | 30962 | 6:37311375 | 30963 | 6:37311766 | 30964 | 6:37311816 |
| 30965 | 6:37311980 | 30966 | 6:37312071 | 30967 | 6:37312238 | 30968 | 6:37312270 | 30969 | 6:37312326 |
| 30970 | 6:37312692 | 30971 | 6:37313215 | 30972 | 6:37313503 | 30973 | 6:37313663 | 30974 | 6:37313742 |
| 30975 | 6:37313954 | 30976 | 6:37314022 | 30977 | 6:37314183 | 30978 | 6:37314291 | 30979 | 6:37315223 |
| 30980 | 6:37315480 | 30981 | 6:37315752 | 30982 | 6:37315774 | 30983 | 6:37316117 | 30984 | 6:37316265 |
| 30985 | 6:37316471 | 30986 | 6:37316594 | 30987 | 6:37316659 | 30988 | 6:37316928 | 30989 | 6:37317087 |
| 30990 | 6:37317153 | 30991 | 6:37317167 | 30992 | 6:37317341 | 30993 | 6:37317695 | 30994 | 6:37318285 |
| 30995 | 6:37318305 | 30996 | 6:37318658 | 30997 | 6:37318743 | 30998 | 6:37318847 | 30999 | 6:37319284 |
| 31000 | 6:37319902 | 31001 | 6:37320093 | 31002 | 6:37321012 | 31003 | 6:37321049 | 31004 | 6:37321441 |
| 31005 | 6:37321485 | 31006 | 6:37322361 | 31007 | 6:37322770 | 31008 | 6:37323838 | 31009 | 6:37323843 |
| 31010 | 6:37324718 | 31011 | 6:37325097 | 31012 | 6:37325189 | 31013 | 6:37326965 | 31014 | 6:37327393 |
| 31015 | 6:37327644 | 31016 | 6:37328014 | 31017 | 6:37328069 | 31018 | 6:37328528 | 31019 | 6:37328898 |
| 31020 | 6:37329042 | 31021 | 6:37329061 | 31022 | 6:37329319 | 31023 | 6:37329413 | 31024 | 6:37329526 |
| 31025 | 6:37329583 | 31027 | 6:37329948 | 31028 | 6:37330186 | 31029 | 6:37330198 | 31031 | 6:37330365 |
| 31034 | 6:37330605 | 31035 | 6:37330677 | 31036 | 6:37331712 | 31037 | 6:37331772 | 31038 | 6:37331921 |
| 31039 | 6:37332914 | 31040 | 6:37333089 | 31041 | 6:37333257 | 31042 | 6:37333646 | 31043 | 6:37335096 |
| 31044 | 6:37335574 | 31045 | 6:37335884 | 31047 | 6:37336176 | 31048 | 6:37336277 | 31049 | 6:37336431 |
| 31050 | 6:37337044 | 31051 | 6:37337343 | 31053 | 6:37337910 | 31054 | 6:37340194 | 31055 | 6:37340475 |
| 31056 | 6:37340937 | 31057 | 6:37341535 | 31058 | 6:37342568 | 31059 | 6:37342705 | 31060 | 6:37343364 |
| 3106 | 6:37343379 | 31062 | 6:37344246 | 31063 | 6:37344303 | 31064 | 6:37344897 | 31065 | 6:37345259 |
| 31066 | 6:37345508 | 31067 | 6:37345931 | 31068 | 6:37346124 | 31069 | 6:37346442 | 31070 | 6:37347130 |
| 3107 | 6:37349144 | 31072 | 6:37350316 | 31073 | 6:37350368 | 31074 | 6:37351499 | 31075 | 6:37351767 |
| 31076 | 6:37352145 | 31077 | 6:37352282 | 31078 | 6:37352820 | 31079 | 6:37353288 | 31080 | 6:37354945 |
| 31081 | 6:37355132 | 31082 | 6:37355369 | 31083 | 6:37356067 | 31084 | 6:37357055 | 31085 | 6:37358828 |
| 31086 | 6:37359765 | 31087 | 6:37360499 | 31088 | 6:37360685 | 31089 | 6:37361449 | 31090 | 6:37361578 |
| 31092 | 6:37363842 | 31093 | 6:37365265 | 31094 | 6:37365744 | 31095 | 6:37366868 | 31096 | 6:37368371 |
| 31097 | 6:37368652 | 31098 | 6:37368670 | 31100 | 6:37369380 | 31101 | 6:37370421 | 31103 | 6:37373261 |
| 31104 | 6:37373314 | 31105 | 6:37374314 | 31106 | 6:37374718 | 31107 | 6:37376009 | 31109 | 6:37377606 |
| 31110 | 6:37377857 | 31112 | 6:37378824 | 31114 | 6:37379507 | 31115 | 6:37379693 | 31116 | 6:37379729 |
| 31117 | 6:37379796 | 31118 | 6:37380161 | 31119 | 6:37380671 | 31120 | 6:37381106 | 31121 | 6:37381616 |
| 31125 | 6:37382177 | 31126 | 6:37383284 | 31128 | 6:37384356 | 31130 | 6:37390341 | 31131 | 6:37390788 |
| 31132 | 6:37391198 | 31133 | 6:37391915 | 31134 | 6:37392465 | 31135 | 6:37392540 | 31136 | 6:37392957 |
| 3113 | 6:37393564 | 31138 | 6:37393949 | 31140 | 6:37395313 | 31141 | 6:37395750 | 31142 | 6:37395838 |
| 31143 | 6:37396789 | 31144 | 6:37398199 | 31146 | 6:37399852 | 31147 | 6:37400741 | 31148 | 6:37400800 |
| 31149 | 6:37402511 | 31150 | 6:37402964 | 31152 | 6:37403224 | 31153 | 6:37406239 | 31154 | 6:37406485 |
| 31155 | 6:37406729 | 31156 | 6:37407653 | 31157 | 6:37412062 | 31158 | 6:37412935 | 31159 | 6:37414352 |
| 31160 | 6:37414365 | 31161 | 6:37414416 | 31162 | 6:37415367 | 31164 | 6:37416649 | 31165 | 6:37417046 |
| 31167 | 6:37418164 | 31168 | 6:37418421 | 31170 | 6:37418449 | 31171 | 6:37420252 | 31173 | 6:37423505 |
| 31174 | 6:37424248 | 31175 | 6:37425030 | 31176 | 6:37429071 | 31177 | 6:37429595 | 31180 | 6:37431101 |
| 31181 | 6:37432458 | 31182 | 6:37432854 | 31183 | 6:37433502 | 31184 | 6:37433926 | 31185 | 6:37434244 |
| 31186 | 6:37435861 | 31187 | 6:37436146 | 31188 | 6:37436347 | 31189 | 6:37436731 | 31190 | 6:37437130 |
| 31191 | 6:37437538 | 31193 | 6:37438248 | 31194 | 6:37438530 | 31195 | 6:37438575 | 31196 | 6:37438599 |
| 31197 | 6:37438993 | 31198 | 6:37439190 | 31200 | 6:37440955 | 31201 | 6:37441274 | 31202 | 6:37441899 |
| 31203 | 6:37442320 | 31204 | 6:37444900 | 31205 | 6:37446552 | 31206 | 6:37448497 | 31207 | 6:37449358 |
| 31208 | 6:37449890 | 31209 | 6:37450921 | 31210 | 6:37453215 | 31211 | 6:37456193 | 31213 | 6:37461999 |
| 31214 | 6:37463282 | 31215 | 6:37463709 | 31217 | 6:37466194 | 31218 | 6:37473453 | 31219 | 6:37477629 |
| 31220 | 6:37479269 | 31221 | 6:37480277 | 31223 | 6:37483537 | 31224 | 6:37489614 | 31226 | 6:37493760 |
| 31227 | 6:37495347 | 31228 | 6:37500943 | 31230 | 6:37505093 | 31231 | 6:37508184 | 31232 | 6:37511610 |
| 31233 | 6:37512672 | 31234 | 6:37512811 | 31235 | 6:37513645 | 31236 | 6:37520035 | 31237 | 6:37521235 |
| 31238 | 6:37522190 | 31239 | 6:37522580 | 31240 | 6:37526812 | 31241 | 6:37527163 | 31242 | 6:37529800 |
| 31244 | 6:37543134 | 31245 | 6:37547826 | 31247 | 6:37559117 | 31250 | 6:37564896 | 31251 | 6:37566268 |
| 31253 | 6:37566504 | 31255 | 6:37573615 | 31257 | 6:37578899 | 31258 | 6:37579956 | 31259 | 6:37580857 |
| 31260 | 6:37585527 | 31261 | 6:37587677 | 31264 | 6:37591326 | 31266 | 6:37592943 | 31267 | 6:37595517 |
| 31269 | 6:37599145 | 31270 | 6:37599699 | 31271 | 6:37602013 | 31272 | 6:37604468 | 31273 | 6:37606625 |
| 31274 | 6:37607139 | 31275 | 6:37608496 | 31276 | 6:37608504 | 31277 | 6:37614039 | 31278 | 6:37614179 |
| 31279 | 6:37617530 | 31280 | 6:37624003 | 31281 | 6:37625923 | 31282 | 6:37627776 | 31283 | 6:37633434 |
| 31284 | 6:37636883 | 31285 | 6:37636900 | 31286 | 6:37637484 | 31290 | 6:37641448 | 31293 | 6:37644691 |
| 31294 | 6:37644915 | 31295 | 6:37645671 | 31296 | 6:37646000 | 31297 | 6:37646499 | 31298 | 6:37646597 |
| 31299 | 6:37652729 | 31300 | 6:37658289 | 31302 | 6:37664673 | 31303 | 6:37666279 | 31304 | 6:37669453 |
| 31305 | 6:37674317 | 31306 | 6:37675400 | 31307 | 6:37679238 | 31308 | 6:37681968 | 31309 | 6:37683699 |
| 31310 | 6:37687844 | 31312 | 6:37690283 | 31313 | 6:37690852 | 31314 | 6:37696619 | 31315 | 6:37697757 |
| 31316 | 6:37698657 | 31317 | 6:37699059 | 31318 | 6:37703461 | 31319 | 6:37704637 | 31320 | 6:37706431 |
| 31321 | 6:37706528 | 31322 | 6:37708048 | 31323 | 6:37708742 | 31324 | 6:37708953 | 31325 | 6:37709524 |
| 31327 | 6:37712519 | 31328 | 6:37712749 | 31329 | 6:37713097 | 31330 | 6:37713686 | 31331 | 6:37713889 |
| 31332 | 6:37719635 | 31333 | 6:37719804 | 31334 | 6:37720524 | 31335 | 6:37727280 | 31336 | 6:37728142 |
| 31337 | 6:37731195 | 31338 | 6:37736960 | 31339 | 6:37738532 | 31340 | 6:37740667 | 31341 | 6:37741893 |
| 31342 | 6:37742839 | 31343 | 6:37743712 | 31344 | 6:37749840 | 31345 | 6:37749955 | 31346 | 6:37751452 |
| 31347 | 6:37752740 | 31348 | 6:37760883 | 31349 | 6:37772675 | 31350 | 6:37773321 | 31351 | 6:37773355 |
| 31352 | 6:37774136 | 31354 | 6:37775491 | 31355 | 6:37775624 | 31356 | 6:37775822 | 31357 | 6:37776401 |
| 31358 | 6:37776428 | 31359 | 6:37776454 | 31360 | 6:37776464 | 31362 | 6:37777056 | 31363 | 6:37777185 |
| 31364 | 6:37778681 | 31365 | 6:37778924 | 31367 | 6:37797233 | 31368 | 6:37802327 | 31369 | 6:37803937 |
| 31370 | 6:37803941 | 31372 | 6:37804729 | 31373 | 6:37805097 | 31374 | 6:37806070 | 31376 | 6:37809966 |
| 31377 | 6:37810505 | 31378 | 6:37810779 | 31379 | 6:37811541 | 31380 | 6:37811600 | 31381 | 6:37811808 |
| 31382 | 6:37811947 | 31383 | 6:37812109 | 31384 | 6:37812521 | 31385 | 6:37813026 | 31386 | 6:37813294 |
| 31387 | 6:37813424 | 31388 | 6:37813572 | 31389 | 6:37813638 | 31390 | 6:37813728 | 31391 | 6:37813964 |
| 31392 | 6:37814265 | 31393 | 6:37814928 | 31394 | 6:37815457 | 31395 | 6:37815489 | 31396 | 6:37815615 |
| 31397 | 6:37815806 | 31398 | 6:37815918 | 31399 | 6:37816011 | 31400 | 6:37816192 | 31401 | 6:37816231 |
| 31402 | 6:37816300 | 31403 | 6:37817310 | 31405 | 6:37817589 | 31406 | 6:37817855 | 31407 | 6:37817875 |
| 31408 | 6:37817910 | 31409 | 6:37818107 | 31410 | 6:37818111 | 31411 | 6:37818195 | 31412 | 6:37818372 |
| 31413 | 6:37819224 | 31414 | 6:37821523 | 31415 | 6:37821595 | 31416 | 6:37821964 | 31417 | 6:37830099 |
| 31418 | 6:37831626 | 31419 | 6:37833439 | 31420 | 6:37838622 | 31421 | 6:37842392 | 31422 | 6:37895331 |
| 31423 | 6:37902755 | 31424 | 6:37905296 | 31425 | 6:37905467 | 31426 | 6:37907357 | 31427 | 6:37908478 |
| 31428 | 6:37908507 | 31429 | 6:37908679 | 31430 | 6:37908861 | 31431 | 6:37908962 | 31432 | 6:37909143 |
| 31433 | 6:37910276 | 31434 | 6:37910464 | 31435 | 6:37910478 | 31436 | 6:37910866 | 31437 | 6:37911814 |
| 31438 | 6:37912116 | 31439 | 6:37913125 | 31440 | 6:37913652 | 31441 | 6:37913727 | 31442 | 6:37913922 |
| 31443 | 6:37913940 | 31444 | 6:37914439 | 31446 | 6:37914513 | 31447 | 6:37914730 | 31448 | 6:37914816 |
| 31449 | 6:37914822 | 31450 | 6:37914832 | 31451 | 6:37915050 | 31452 | 6:37915308 | 31453 | 6:37916419 |
| 31454 | 6:37917366 | 31455 | 6:37917421 | 31456 | 6:37917796 | 31457 | 6:37918546 | 31458 | 6:37918824 |
| 31459 | 6:37919045 | 31460 | 6:37919095 | 31461 | 6:37919120 | 31462 | 6:37919520 | 31463 | 6:37919631 |
| 31464 | 6:37919661 | 31465 | 6:37919853 | 31466 | 6:37919862 | 31467 | 6:37920305 | 31468 | 6:37920538 |
| 31469 | 6:37920551 | 31470 | 6:37920666 | 31471 | 6:37921510 | 31472 | 6:37921603 | 31474 | 6:37921724 |
| 31476 | 6:37926600 | 31477 | 6:37926960 | 31478 | 6:37928156 | 31479 | 6:37928479 | 31480 | 6:37929065 |
| 31481 | 6:37929104 | 31482 | 6:37929184 | 31483 | 6:37929689 | 31484 | 6:37929786 | 31485 | 6:37930046 |
| 31487 | 6:37930293 | 31488 | 6:37930672 | 31489 | 6:37930781 | 31490 | 6:37930868 | 31491 | 6:37930889 |
| 31492 | 6:37931089 | 31493 | 6:37931216 | 31494 | 6:37931298 | 31495 | 6:37931621 | 31496 | 6:37931688 |
| 31497 | 6:37931726 | 31498 | 6:37931903 | 31499 | 6:37932170 | 31500 | 6:37932197 | 31501 | 6:37932241 |
| 31502 | 6:37932371 | 31503 | 6:37932377 | 31504 | 6:37932412 | 31505 | 6:37932434 | 31506 | 6:37932563 |
| 31508 | 6:37932772 | 31509 | 6:37932778 | 31510 | 6:37932960 | 31511 | 6:37933250 | 31512 | 6:37933352 |
| 31513 | 6:37933965 | 31514 | 6:37934068 | 31515 | 6:37934272 | 31516 | 6:37934993 | 31517 | 6:37935069 |
| 31518 | 6:37935108 | 31519 | 6:37935168 | 31520 | 6:37935527 | 31521 | 6:37935577 | 31522 | 6:37935748 |
| 31523 | 6:37936044 | 31524 | 6:37936087 | 31525 | 6:37936692 | 31526 | 6:37938294 | 31529 | 6:37942096 |
| 31530 | 6:37942170 | 31531 | 6:37942290 | 31532 | 6:37942944 | 31533 | 6:37943635 | 31534 | 6:37943860 |
| 31536 | 6:37944478 | 31537 | 6:37944482 | 31538 | 6:37944496 | 31539 | 6:37944573 | 31540 | 6:37944631 |
| 31541 | 6:37944681 | 31542 | 6:37944704 | 31543 | 6:37945333 | 31544 | 6:37945482 | 31545 | 6:37945820 |
| 31546 | 6:37945849 | 31547 | 6:37946012 | 31548 | 6:37946384 | 31549 | 6:37953254 | 31550 | 6:37953301 |
| 31551 | 6:37953591 | 31552 | 6:37954699 | 31553 | 6:37954789 | 31554 | 6:37954873 | 31555 | 6:37955372 |
| 31556 | 6:37956368 | 31557 | 6:37956390 | 31558 | 6:37959360 | 31559 | 6:37960313 | 31560 | 6:37961491 |
| 31561 | 6:37961553 | 31562 | 6:37962093 | 31563 | 6:37962887 | 31564 | 6:37963346 | 31565 | 6:37963525 |
| 31566 | 6:37965259 | 31567 | 6:37965489 | 31568 | 6:37966382 | 31569 | 6:37968595 | 31570 | 6:37970172 |
| 31571 | 6:37970860 | 31572 | 6:37971218 | 31573 | 6:37971544 | 31574 | 6:37971810 | 31575 | 6:37971997 |
| 31576 | 6:37972173 | 31577 | 6:37972335 | 31578 | 6:37972414 | 31579 | 6:37972642 | 31580 | 6:37972906 |
| 31581 | 6:37973011 | 31582 | 6:37973508 | 31583 | 6:37973804 | 31584 | 6:37974302 | 31586 | 6:37974407 |
| 31587 | 6:37974974 | 31588 | 6:37976818 | 31589 | 6:37978037 | 31590 | 6:37978995 | 31591 | 6:37979083 |
| 31592 | 6:37981093 | 31593 | 6:37981125 | 31594 | 6:37983153 | 31595 | 6:37984808 | 31596 | 6:37984948 |
| 31597 | 6:37985243 | 31598 | 6:37987337 | 31599 | 6:37987503 | 31600 | 6:37989831 | 31601 | 6:37990674 |
| 31602 | 6:37990808 | 31603 | 6:37991751 | 31604 | 6:37992856 | 31605 | 6:37993125 | 31606 | 6:37993517 |
| 31607 | 6:37993754 | 31608 | 6:37994046 | 31609 | 6:37996786 | 31610 | 6:37996859 | 31611 | 6:37997160 |
| 31612 | 6:37997516 | 31613 | 6:37998187 | 31614 | 6:37998452 | 31615 | 6:37998592 | 31616 | 6:37998656 |
| 31617 | 6:37998696 | 31618 | 6:37999740 | 31619 | 6:37999825 | 31620 | 6:37999842 | 31621 | 6:38000343 |
| 31622 | 6:38000353 | 31623 | 6:38001392 | 31624 | 6:38001622 | 31625 | 6:38002117 | 31626 | 6:38002596 |
| 31627 | 6:38004124 | 31628 | 6:38004325 | 31629 | 6:38005805 | 31630 | 6:38007975 | 31631 | 6:38014109 |
| 31632 | 6:38016521 | 31633 | 6:38017827 | 31634 | 6:38017866 | 31635 | 6:38021694 | 31636 | 6:38022226 |
| 31637 | 6:38022305 | 31638 | 6:38023218 | 31639 | 6:38023327 | 31640 | 6:38023545 | 31641 | 6:38023613 |
| 31642 | 6:38023673 | 31643 | 6:38023878 | 31644 | 6:38024011 | 31645 | 6:38024702 | 31646 | 6:38024768 |
| 31647 | 6:38025411 | 31648 | 6:38025684 | 31649 | 6:38025734 | 31650 | 6:38026545 | 31651 | 6:38027078 |
| 31652 | 6:38027106 | 31653 | 6:38027129 | 31654 | 6:38031018 | 31655 | 6:38032275 | 31656 | 6:38033896 |
| 31658 | 6:38035001 | 31659 | 6:38035414 | 31660 | 6:38035460 | 31661 | 6:38035561 | 31662 | 6:38035568 |
| 31663 | 6:38035625 | 31664 | 6:38035705 | 31665 | 6:38036061 | 31666 | 6:38036351 | 31667 | 6:38036588 |
| 31668 | 6:38036594 | 31669 | 6:38036635 | 31670 | 6:38036660 | 31671 | 6:38036882 | 31672 | 6:38037099 |
| 31673 | 6:38037535 | 31674 | 6:38037724 | 31675 | 6:38037768 | 31676 | 6:38038091 | 31677 | 6:38038189 |
| 31678 | 6:38038404 | 31679 | 6:38038608 | 31680 | 6:38039088 | 31681 | 6:38039492 | 31682 | 6:38040456 |
| 31683 | 6:38040488 | 31684 | 6:38040529 | 31685 | 6:38040769 | 31686 | 6:38040934 | 31687 | 6:38041234 |
| 31688 | 6:38041593 | 31689 | 6:38041633 | 31690 | 6:38041715 | 31691 | 6:38042205 | 31692 | 6:38042337 |
| 31693 | 6:38043977 | 31694 | 6:38044618 | 31695 | 6:38044876 | 31696 | 6:38044898 | 31697 | 6:38045727 |
| 31698 | 6:38046428 | 31699 | 6:38048735 | 31700 | 6:38051406 | 31701 | 6:38051509 | 31702 | 6:38052049 |
| 31703 | 6:38052245 | 31704 | 6:38054733 | 31706 | 6:38063490 | 31707 | 6:38064317 | 31708 | 6:38065703 |
| 31709 | 6:38066309 | 31710 | 6:38067289 | 31711 | 6:38067376 | 31712 | 6:38067855 | 31713 | 6:38068311 |
| 31714 | 6:38068956 | 31715 | 6:38069662 | 31716 | 6:38070361 | 31717 | 6:38070496 | 31718 | 6:38070882 |
| 31719 | 6:38071318 | 31720 | 6:38071414 | 31721 | 6:38071547 | 31722 | 6:38073914 | 31723 | 6:38076161 |
| 31724 | 6:38076905 | 31725 | 6:38077237 | 31726 | 6:38077323 | 31728 | 6:38078774 | 31729 | 6:38079456 |
| 31730 | 6:38079499 | 31731 | 6:38082355 | 31732 | 6:38085803 | 31733 | 6:38087237 | 31734 | 6:38087636 |
| 31735 | 6:38089862 | 31736 | 6:38094533 | 31737 | 6:38097987 | 31739 | 6:38107020 | 31740 | 6:38108493 |
| 31742 | 6:38114043 | 31743 | 6:38114047 | 31744 | 6:38114717 | 31745 | 6:38114803 | 31746 | 6:38114808 |
| 31747 | 6:38115023 | 31748 | 6:38118015 | 31749 | 6:38122903 | 31750 | 6:38123039 | 31751 | 6:38127700 |
| 31752 | 6:38131158 | 31753 | 6:38132133 | 31754 | 6:38132688 | 31755 | 6:38135706 | 31756 | 6:38137640 |
| 31757 | 6:38137799 | 31758 | 6:38138017 | 31759 | 6:38138089 | 31760 | 6:38139495 | 31761 | 6:38139563 |
| 31762 | 6:38139762 | 31763 | 6:38140108 | 31764 | 6:38140123 | 31765 | 6:38140536 | 31766 | 6:38140681 |
| 31767 | 6:38141284 | 31768 | 6:38141304 | 31769 | 6:38141629 | 31770 | 6:38141696 | 31771 | 6:38141833 |
| 31772 | 6:38142148 | 31773 | 6:38142807 | 31774 | 6:38142835 | 31775 | 6:38142840 | 31776 | 6:38144120 |
| 31777 | 6:38144195 | 31778 | 6:38144291 | 31779 | 6:38144317 | 31780 | 6:38144587 | 31781 | 6:38156378 |
| 31782 | 6:38156469 | 31783 | 6:38156591 | 31784 | 6:38157637 | 31785 | 6:38157684 | 31786 | 6:38158107 |
| 31787 | 6:38158113 | 31791 | 6:38199691 | 31792 | 6:38202499 | 31793 | 6:38211198 | 31795 | 6:38227779 |
| 31797 | 6:38237566 | 31798 | 6:38237695 | 31799 | 6:38247802 | 31800 | 6:38252776 | 31805 | 6:38278657 |
| 31806 | 6:38278666 | 31807 | 6:38278782 | 31808 | 6:38279030 | 31809 | 6:38281274 | 31814 | 6:38318628 |
| 31817 | 6:38375856 | 31820 | 6:38396789 | 31823 | 6:38409609 | 31825 | 6:38433243 | 31827 | 6:38499580 |
| 31828 | 6:38500871 | 31829 | 6:38506361 | 31830 | 6:38508464 | 31832 | 6:38519250 | 31833 | 6:38525790 |
| 31834 | 6:38535911 | 31835 | 6:38540318 | 31838 | 6:38541639 | 31840 | 6:38542150 | 31844 | 6:38543990 |
| 31848 | 6:38546646 | 31849 | 6:38548060 | 31850 | 6:38548198 | 31854 | 6:38549044 | 31856 | 6:38549206 |
| 31858 | 6:38549703 | 31859 | 6:38550162 | 31869 | 6:38553181 | 31881 | 6:38556220 | 31895 | 6:38562229 |
| 31896 | 6:38563063 | 31898 | 6:38565084 | 31899 | 6:38566242 | 31900 | 6:38570744 | 31901 | 6:38575762 |
| 31903 | 6:38584420 | 31904 | 6:38584686 | 31905 | 6:38588303 | 31906 | 6:38599441 | 31907 | 6:38600028 |
| 31908 | 6:38600235 | 31909 | 6:38602696 | 31910 | 6:38617268 | 31911 | 6:38617890 | 31912 | 6:38618593 |
| 31913 | 6:38619398 | 31914 | 6:38619754 | 31915 | 6:38619795 | 31916 | 6:38620014 | 31917 | 6:38620218 |
| 31918 | 6:38621930 | 31919 | 6:38624000 | 31920 | 6:38624126 | 31921 | 6:38644939 | 31923 | 6:38695951 |
| 31924 | 6:38697400 | 31928 | 6:38789984 | 31929 | 6:38798194 | 31933 | 6:38854750 | 31942 | 6:38907049 |
| 31943 | 6:38918524 | 31945 | 6:38919029 | 31946 | 6:38929469 | 31947 | 6:38933534 | 31948 | 6:38934821 |
| 31950 | 6:38963229 | 31951 | 6:38973635 | 31952 | 6:38973995 | 31953 | 6:38980873 | 31954 | 6:39030259 |
| 31956 | 6:39082331 | 31957 | 6:39109137 | 31962 | 6:39181262 | 31966 | 6:39254458 | 31968 | 6:39280700 |
| 31974 | 6:39437165 | 31977 | 6:39518939 | 31978 | 6:39532147 | 31979 | 6:39559751 | 31982 | 6:39574982 |
| 31984 | 6:39595691 | 31985 | 6:39595731 | 31988 | 6:39652421 | 31992 | 6:39721697 | 31994 | 6:39730068 |
| 31995 | 6:39733610 | 31996 | 6:39737879 | 31998 | 6:39755798 | 31999 | 6:39756006 | 32002 | 6:39760650 |
| 32003 | 6:39772834 | 32004 | 6:39772963 | 32005 | 6:39773133 | 32007 | 6:39797151 | 32009 | 6:39825526 |
| 32011 | 6:39858450 | 32012 | 6:39874821 | 32013 | 6:39885647 | 32026 | 6:40072649 | 32030 | 6:40130949 |
| 32032 | 6:40203593 | 32035 | 6:40239109 | 32036 | 6:40248052 | 32042 | 6:40338033 | 32047 | 6:40400197 |
| 32048 | 6:40432849 | 32049 | 6:40456680 | 32052 | 6:40565574 | 32055 | 6:40599155 | 32058 | 6:40636686 |
| 32059 | 6:40662051 | 32060 | 6:40672232 | 32061 | 6:40689118 | 32062 | 6:40696320 | 32085 | 6:40731922 |
| 32089 | 6:40758035 | 32093 | 6:40780850 | 32094 | 6:40808612 | 32098 | 6:40836283 | 32102 | 6:40911873 |
| 32103 | 6:40920367 | 32104 | 6:40921708 | 32105 | 6:40933517 | 32106 | 6:40935693 | 32107 | 6:40935713 |
| 32108 | 6:40935857 | 32109 | 6:40935890 | 32110 | 6:40936031 | 32111 | 6:40936207 | 32112 | 6:40936326 |
| 32113 | 6:40936831 | 32114 | 6:40936837 | 32115 | 6:40937031 | 32116 | 6:40937441 | 32117 | 6:40942588 |
| 32120 | 6:40984868 | 32121 | 6:40988645 | 32122 | 6:40988913 | 32123 | 6:40999090 | 32124 | 6:41003445 |
| 32125 | 6:41004894 | 32126 | 6:41029896 | 32127 | 6:41033792 | 32128 | 6:41049370 | 32129 | 6:41049496 |
| 32130 | 6:41049859 | 32131 | 6:41049914 | 32132 | 6:41050127 | 32133 | 6:41050140 | 32135 | 6:41050862 |
| 32136 | 6:41051120 | 32137 | 6:41053625 | 32138 | 6:41061635 | 32139 | 6:41073550 | 32140 | 6:41079462 |
| 32142 | 6:41088062 | 32143 | 6:41090326 | 32144 | 6:41096500 | 32145 | 6:41107449 | 32146 | 6:41107836 |
| 32147 | 6:41108192 | 32148 | 6:41108818 | 32149 | 6:41108912 | 32150 | 6:41108926 | 32151 | 6:41109137 |
| 32152 | 6:41109217 | 32153 | 6:41109350 | 32155 | 6:41150218 | 32156 | 6:41169318 | 32157 | 6:41170432 |
| 32158 | 6:41173967 | 32161 | 6:41185741 | 32162 | 6:41204988 | 32169 | 6:41306166 | 32170 | 6:41306273 |
| 32171 | 6:41306284 | 32172 | 6:41306424 | 32173 | 6:41308067 | 32174 | 6:41331258 | 32179 | 6:41577847 |
| 32180 | 6:41592300 | 32181 | 6:41677493 | 32188 | 6:41868518 | 32195 | 6:42004652 | 32203 | 6:42037316 |
| 32209 | 6:42163671 | 32216 | 6:42441818 | 32220 | 6:42507082 | 32221 | 6:42510330 | 32227 | 6:42559603 |
| 32229 | 6:42665842 | 32230 | 6:42672683 | 32231 | 6:42722868 | 32232 | 6:42724776 | 32233 | 6:42728138 |
| 32234 | 6:42741244 | 32235 | 6:42741447 | 32236 | 6:42758331 | 32241 | 6:42837669 | 32263 | 6:42974326 |
| 32284 | 6:43317249 | 32296 | 6:43494887 | 32298 | 6:43544441 | 32303 | 6:43601154 | 32309 | 6:43679917 |
| 32318 | 6:43822090 | 32319 | 6:43822403 | 32320 | 6:43829217 | 32323 | 6:43886543 | 32326 | 6:43944737 |
| 32330 | 6:43983354 | 32331 | 6:44003666 | 32332 | 6:44026498 | 32336 | 6:44072335 | 32339 | 6:44149286 |
| 32343 | 6:44276184 | 32360 | 6:44628911 | 32362 | 6:44678042 | 32363 | 6:44685469 | 32364 | 6:44686809 |
| 32365 | 6:44689379 | 32366 | 6:44690759 | 32368 | 6:44694506 | 32369 | 6:44694605 | 32370 | 6:44695255 |
| 32371 | 6:44695433 | 32372 | 6:44695563 | 32373 | 6:44695784 | 32374 | 6:44695872 | 32375 | 6:44695978 |
| 32376 | 6:44695987 | 32377 | 6:44696022 | 32378 | 6:44696578 | 32379 | 6:44696796 | 32380 | 6:44696931 |
| 32381 | 6:44696955 | 32382 | 6:44698160 | 32383 | 6:44704061 | 32384 | 6:44704410 | 32385 | 6:44705240 |
| 32386 | 6:44706199 | 32387 | 6:44706279 | 32388 | 6:44709016 | 32389 | 6:44709754 | 32390 | 6:44710003 |
| 32391 | 6:44710237 | 32392 | 6:44710415 | 32393 | 6:44710515 | 32394 | 6:44711099 | 32395 | 6:44712295 |
| 32396 | 6:44735374 | 32397 | 6:44736099 | 32398 | 6:44739055 | 32399 | 6:44742360 | 32400 | 6:44747933 |
| 32401 | 6:44748956 | 32403 | 6:44775326 | 32404 | 6:44777357 | 32405 | 6:44784742 | 32406 | 6:44787261 |
| 32407 | 6:44791513 | 32408 | 6:44791744 | 32409 | 6:44797875 | 32410 | 6:44809081 | 32411 | 6:44810108 |
| 32412 | 6:44818322 | 32413 | 6:44819710 | 32414 | 6:44820115 | 32415 | 6:44823097 | 32416 | 6:44825630 |
| 32418 | 6:44828213 | 32419 | 6:44831889 | 32420 | 6:44857705 | 32421 | 6:44864378 | 32422 | 6:44873514 |
| 32423 | 6:44873843 | 32424 | 6:44874186 | 32425 | 6:44877670 | 32426 | 6:44883344 | 32427 | 6:44893464 |
| 32428 | 6:44904257 | 32429 | 6:44905170 | 32430 | 6:44909660 | 32431 | 6:44914656 | 32432 | 6:44920744 |
| 32433 | 6:44931581 | 32434 | 6:44934298 | 32435 | 6:44934689 | 32437 | 6:44983137 | 32439 | 6:45018531 |
| 32440 | 6:45018882 | 32441 | 6:45029390 | 32442 | 6:45029425 | 32443 | 6:45029626 | 32444 | 6:45030486 |
| 32445 | 6:45030599 | 32446 | 6:45030709 | 32447 | 6:45030774 | 32448 | 6:45030789 | 32449 | 6:45031327 |
| 32450 | 6:45031721 | 32451 | 6:45031956 | 32452 | 6:45032017 | 32453 | 6:45032153 | 32454 | 6:45033264 |
| 32455 | 6:45037315 | 32456 | 6:45037561 | 32457 | 6:45037753 | 32458 | 6:45037798 | 32459 | 6:45038091 |
| 32460 | 6:45038647 | 32461 | 6:45038676 | 32462 | 6:45038869 | 32463 | 6:45040756 | 32464 | 6:45041754 |
| 32465 | 6:45043489 | 32466 | 6:45043654 | 32467 | 6:45043859 | 32468 | 6:45043984 | 32469 | 6:45044149 |
| 32470 | 6:45045596 | 32471 | 6:45048213 | 32472 | 6:45048323 | 32473 | 6:45049586 | 32474 | 6:45053716 |
| 32475 | 6:45054332 | 32476 | 6:45055326 | 32477 | 6:45057776 | 32478 | 6:45057805 | 32479 | 6:45058034 |
| 32480 | 6:45058273 | 32481 | 6:45077729 | 32485 | 6:45135036 | 32487 | 6:45183436 | 32489 | 6:45185710 |
| 32490 | 6:45190122 | 32491 | 6:45205382 | 32492 | 6:45213648 | 32497 | 6:45274484 | 32500 | 6:45322366 |
| 32502 | 6:45325845 | 32505 | 6:45364629 | 32518 | 6:45583636 | 32519 | 6:45595528 | 32526 | 6:45885499 |
| 32569 | 6:46705772 | 32576 | 6:46946921 | 32580 | 6:47016991 | 32586 | 6:47147481 | 32587 | 6:47164702 |
| 32591 | 6:47219584 | 32592 | 6:47256173 | 32594 | 6:47317499 | 32596 | 6:47365064 | 32599 | 6:47424087 |
| 32602 | 6:47570425 | 32614 | 6:47825353 | 32615 | 6:47843766 | 32616 | 6:47866852 | 32621 | 6:47989356 |
| 32623 | 6:48030364 | 32626 | 6:48149995 | 32648 | 6:48893818 | 32652 | 6:49019297 | 32655 | 6:49094600 |
| 32662 | 6:49295440 | 32679 | 6:49701147 | 32694 | 6:49848545 | 32696 | 6:49878456 | 32701 | 6:50009636 |
| 32703 | 6:50090470 | 32704 | 6:50126302 | 32709 | 6:50252554 | 32716 | 6:50385741 | 32717 | 6:50412585 |
| 32719 | 6:50455132 | 32724 | 6:50672369 | 32729 | 6:50779278 | 32731 | 6:50856440 | 32748 | 6:51325072 |
| 32750 | 6:51362285 | 32759 | 6:51740645 | 32778 | 6:52250315 | 32780 | 6:52275948 | 32781 | 6:52320441 |
| 32782 | 6:52339267 | 32783 | 6:52339325 | 32784 | 6:52347811 | 32785 | 6:52347899 | 32786 | 6:52347995 |
| 32787 | 6:52348073 | 32788 | 6:52348287 | 32789 | 6:52348292 | 32790 | 6:52348643 | 32791 | 6:52348674 |
| 32792 | 6:52349023 | 32793 | 6:52349157 | 32794 | 6:52349544 | 32795 | 6:52349562 | 32796 | 6:52349794 |
| 32797 | 6:52350003 | 32798 | 6:52350227 | 32799 | 6:52350240 | 32800 | 6:52351181 | 32801 | 6:52351388 |
| 32802 | 6:52352446 | 32817 | 6:52645662 | 32818 | 6:52671724 | 32825 | 6:52864424 | 32829 | 6:52954024 |
| 32831 | 6:53024244 | 32842 | 6:53220820 | 32846 | 6:53285747 | 32847 | 6:53314886 | 32850 | 6:53397301 |
| 32853 | 6:53471919 | 32862 | 6:53752557 | 32873 | 6:54004873 | 32874 | 6:54031927 | 32877 | 6:54086328 |
| 32879 | 6:54185052 | 32897 | 6:54783996 | 32913 | 6:55303674 | 32920 | 6:55450495 | 32941 | 6:55514144 |
| 32976 | 6:56317455 | 32994 | 6:56900473 | 33006 | 6:57179884 | 33012 | 6:57260380 | 33031 | 6:57842883 |
| 33035 | 6:57984344 | 33039 | 6:58077967 | 33066 | 6:58746181 | 33077 | 6:59130010 | 33095 | 6:59579872 |
| 33097 | 6:59716766 | 33111 | 6:59875075 | 33114 | 6:59907442 | 33122 | 6:60085027 | 33129 | 6:60257426 |
| 33137 | 6:60400323 | 33155 | 6:60802613 | 33168 | 6:61185257 | 33176 | 6:61390096 | 33183 | 6:61487026 |
| 33204 | 6:62234468 | 33223 | 6:62814916 | 33224 | 6:62862206 | 33225 | 6:62889960 | 33227 | 6:62925583 |
| 33238 | 6:63122811 | 33240 | 6:63170295 | 33242 | 6:63209301 | 33244 | 6:63244734 | 33260 | 6:63836353 |
| 33307 | 6:65456807 | 33308 | 6:65520848 | 33338 | 6:66507621 | 33350 | 6:66896326 | 33361 | 6:67256076 |
| 33376 | 6:67767947 | 33381 | 6:67991798 | 33394 | 6:68331252 | 33397 | 6:68416278 | 33418 | 6:68896760 |
| 33422 | 6:69030825 | 33433 | 6:69252356 | 33435 | 6:69346909 | 33444 | 6:69724839 | 33474 | 6:70673035 |
| 33478 | 6:70789469 | 33484 | 6:70978405 | 33516 | 6:72022899 | 33530 | 6:72349944 | 33534 | 6:72385883 |
| 33535 | 6:72419275 | 33544 | 6:72713559 | 33582 | 6:74091185 | 33584 | 6:74174913 | 33607 | 6:75066888 |
| 33608 | 6:75102649 | 33667 | 6:76880221 | 33698 | 6:77967685 | 33734 | 6:79446058 | 33751 | 6:80043649 |
| 33764 | 6:80444445 | 33798 | 6:81507132 | 33851 | 6:83098669 | 33875 | 6:83547735 | 33897 | 6:83979787 |
| 33921 | 6:84628765 | 33933 | 6:85067620 | 33936 | 6:85092523 | 33946 | 6:85177598 | 33953 | 6:85422786 |
| 33964 | 6:85548797 | 34000 | 6:86388176 | 34002 | 6:86478657 | 34055 | 6:87399244 | 34089 | 6:88024804 |
| 34126 | 6:89007570 | 34128 | 6:89048060 | 34273 | 6:91629835 | 34306 | 6:92470502 | 34307 | 6:92504637 |
| 34410 | 6:94755352 | 34412 | 6:94776615 | 34452 | 6:96090013 | 34453 | 6:96097237 | 34478 | 6:96544331 |
| 34596 | 6:98671599 | 34598 | 6:98696365 | 34635 | 6:99753620 | 34640 | 6:99898377 | 34644 | 6:100026934 |
| 34702 | 6:101156203 | 34718 | 6:101492004 | 34738 | 6:101904289 | 34742 | 6:101951985 | 34771 | 6:102466503 |
| 34774 | 6:102525990 | 34781 | 6:102622674 | 34936 | 6:105596054 | 35002 | 6:107330690 | 35294 | 6:113769965 |
| 35299 | 6:113826996 | 35354 | 6:114785574 | 35370 | 6:115107065 | 35390 | 6:115648265 | 35391 | 6:115670310 |
| 35405 | 6:115976356 | 35426 | 6:116325904 | 35456 | 6:117124190 | 35476 | 7:210754 | 35491 | 7:521674 |
| 35511 | 7:987326 | 35515 | 7:1068152 | 35516 | 7:1068170 | 35638 | 7:3108200 | 35680 | 7:3821837 |
| 35729 | 7:5027580 | 35741 | 7:5125016 | 35859 | 7:6346703 | 35871 | 7:6483779 | 36139 | 7:13283360 |
| 36142 | 7:13315974 | 36174 | 7:13946048 | 36270 | 7:15409865 | 36316 | 7:16530067 | 36366 | 7:17643702 |
| 36378 | 7:17996900 | 36554 | 7:21064478 | 36624 | 7:22566377 | 36628 | 7:22594727 | 36656 | 7:23523707 |
| 36685 | 7:24072015 | 36686 | 7:24142269 | 36697 | 7:24434170 | 36698 | 7:24436989 | 36816 | 7:26920631 |
| 36863 | 7:27929188 | 36943 | 7:29802791 | 36954 | 7:30036916 | 36957 | 7:30105269 | 37015 | 7:31343335 |
| 37063 | 7:32305922 | 37066 | 7:32334690 | 37099 | 7:33229945 | 37111 | 7:33525039 | 37122 | 7:33761341 |
| 37149 | 7:34411840 | 37156 | 7:34553590 | 37176 | 7:35028517 | 37430 | 7:40687768 | 37528 | 7:43355224 |
| 37570 | 7:44672168 | 37748 | 7:48982387 | 37783 | 7:50794543 | 37816 | 7:52176483 | 37833 | 7:52224828 |
| 37838 | 7:52249785 | 38015 | 7:56078682 | 38041 | 7:56774182 | 38048 | 7:56997351 | 38071 | 7:57843022 |
| 38075 | 7:57875535 | 38208 | 7:60668104 | 38263 | 7:61497597 | 38359 | 7:63355823 | 38427 | 7:65211026 |
| 38428 | 7:65229182 | 38429 | 7:65236569 | 38430 | 7:65246212 | 38431 | 7:65252605 | 38470 | 7:66072980 |
| 38560 | 7:67858059 | 38734 | 7:72087070 | 38741 | 7:72254021 | 38780 | 7:73605674 | 38781 | 7:73607632 |
| 38782 | 7:73637074 | 38785 | 7:73667360 | 38867 | 7:75759697 | 38875 | 7:75832195 | 38885 | 7:76174417 |
| 38922 | 7:77054436 | 38942 | 7:77465055 | 38993 | 7:78445222 | 38999 | 7:78709632 | 39099 | 7:80473773 |
| 39124 | 7:80863489 | 39132 | 7:81093522 | 39153 | 7:81316371 | 39179 | 7:81968933 | 39185 | 7:82214691 |
| 39191 | 7:82275842 | 39194 | 7:82369878 | 39298 | 7:85032366 | 39351 | 7:86627973 | 39378 | 7:87376691 |
| 39402 | 7:88102064 | 39416 | 7:88541691 | 39453 | 7:89335556 | 39457 | 7:89404622 | 39489 | 7:90218611 |
| 39512 | 7:90622467 | 39514 | 7:90688919 | 39518 | 7:90701213 | 39520 | 7:90747139 | 39521 | 7:90755504 |
| 39522 | 7:90756120 | 39526 | 7:90805595 | 39527 | 7:90807032 | 39528 | 7:90807192 | 39529 | 7:90807464 |
| 39530 | 7:90807690 | 39531 | 7:90807800 | 39532 | 7:90807968 | 39533 | 7:90808002 | 39534 | 7:90808188 |
| 39535 | 7:90808858 | 39536 | 7:90813061 | 39537 | 7:90813398 | 39539 | 7:90818194 | 39540 | 7:90818916 |
| 39541 | 7:90819613 | 39542 | 7:90820830 | 39543 | 7:90821093 | 39544 | 7:90821542 | 39545 | 7:90821986 |
| 39546 | 7:90822179 | 3954 | 7:90822452 | 39548 | 7:90822601 | 39549 | 7:90823320 | 39550 | 7:90824804 |
| 39551 | 7:90825013 | 39552 | 7:90825646 | 39553 | 7:90825779 | 39554 | 7:90825887 | 39555 | 7:90827178 |
| 39557 | 7:90831014 | 39562 | 7:90842614 | 39567 | 7:90851664 | 39571 | 7:90889577 | 39597 | 7:91489258 |
| 39602 | 7:91611232 | 39603 | 7:91625156 | 39604 | 7:91625242 | 39605 | 7:91625323 | 39606 | 7:91625348 |
| 39609 | 7:91710576 | 39613 | 7:91786432 | 39615 | 7:91796723 | 39616 | 7:91818525 | 39624 | 7:91975067 |
| 39702 | 7:94438004 | 39707 | 7:94529183 | 39744 | 7:95454269 | 39752 | 7:95607084 | 39792 | 7:96165472 |
| 39942 | 7:96409276 | 39956 | 7:96720179 | 39962 | 7:96807889 | 39981 | 7:97434118 | 39990 | 7:97603739 |
| 40255 | 7:104401635 | 40270 | 7:104812484 | 40304 | 7:105679720 | 40323 | 7:106127282 | 40332 | 7:106311771 |
| 40335 | 7:106392644 | 40416 | 7:108497744 | 40421 | 7:108592553 | 40429 | 7:108781177 | 40437 | 7:108939690 |
| 40523 | 8:905854 | 40531 | 8:972589 | 40654 | 8:3953653 | 40665 | 8:4218936 | 40702 | 8:5114851 |
| 40704 | 8:5164202 | 40769 | 8:6831018 | 41003 | 8:12159615 | 41129 | 8:15805194 | 41141 | 8:16218371 |
| 41181 | 8:17034771 | 41209 | 8:17591195 | 41218 | 8:17834202 | 41244 | 8:18677218 | 41417 | 8:23907022 |
| 41564 | 8:25653708 | 41594 | 8:26211730 | 41629 | 8:27285102 | 41727 | 8:29969758 | 41835 | 8:33203967 |
| 41881 | 8:34438846 | 41903 | 8:35034999 | 41929 | 8:35568258 | 41956 | 8:36096463 | 41973 | 8:36469080 |
| 41974 | 8:36489911 | 42016 | 8:37579398 | 42038 | 8:38343034 | 42071 | 8:39659087 | 42104 | 8:40219724 |
| 42149 | 8:41244408 | 42172 | 8:41710832 | 42173 | 8:41735284 | 42187 | 8:42123936 | 42190 | 8:42204721 |
| 42208 | 8:42686594 | 42212 | 8:42720374 | 42216 | 8:42880518 | 42241 | 8:43615936 | 42271 | 8:44484817 |
| 42312 | 8:45608581 | 42314 | 8:45672590 | 42329 | 8:45953160 | 42330 | 8:45985343 | 42332 | 8:46013064 |
| 42395 | 8:47420937 | 42418 | 8:47998648 | 42466 | 8:49248066 | 42519 | 8:50846856 | 42524 | 8:50995576 |
| 42565 | 8:52018600 | 42588 | 8:52629220 | 42591 | 8:52674907 | 42603 | 8:52986604 | 42661 | 8:54554681 |
| 42683 | 8:55077468 | 42694 | 8:55493961 | 42765 | 8:57313074 | 42849 | 8:59626198 | 42853 | 8:59747078 |
| 42916 | 8:61134883 | 42918 | 8:61212042 | 42919 | 8:61235958 | 42922 | 8:61366316 | 42925 | 8:61452677 |
| 43033 | 8:64222797 | 43049 | 8:64876145 | 43067 | 8:65260788 | 43080 | 8:65506614 | 43087 | 8:65773835 |
| 43124 | 8:66668352 | 43200 | 8:68399674 | 43235 | 8:69120902 | 43284 | 8:70429214 | 43306 | 8:70893052 |
| 43311 | 8:70996588 | 43380 | 8:72930313 | 43454 | 8:74772105 | 43457 | 8:74801687 | 43527 | 8:76729847 |
| 43563 | 8:77557071 | 43702 | 8:81553047 | 43722 | 8:82063613 | 43764 | 8:82948559 | 43831 | 8:84602590 |
| 43865 | 8:85540348 | 43883 | 8:85967740 | 43942 | 8:87290730 | 43979 | 8:88449390 | 43993 | 8:88666966 |
| 44077 | 8:90752165 | 44176 | 8:93011122 | 44205 | 8:93906534 | 44262 | 8:95928376 | 44268 | 8:96045608 |
| 44269 | 8:96053216 | 44574 | 8:102774503 | 44648 | 8:104180690 | 44652 | 8:104213734 | 44749 | 8:106594158 |
| 44887 | 8:110915706 | 44899 | 8:111410824 | 45035 | 9:2664478 | 45036 | 9:2667437 | 45037 | 9:2675248 |
| 45038 | 9:2678406 | 45039 | 9:2679109 | 45040 | 9:2681750 | 45041 | 9:2685486 | 45079 | 9:3897414 |
| 45192 | 9:7600098 | 45344 | 9:10670291 | 45354 | 9:10721590 | 45379 | 9:10940149 | 45383 | 9:11053216 |
| 45423 | 9:12009299 | 45432 | 9:12198197 | 45496 | 9:13287682 | 45535 | 9:14277874 | 45589 | 9:15430684 |
| 45660 | 9:16629763 | 45662 | 9:16687402 | 45664 | 9:16727064 | 45676 | 9:17031856 | 45707 | 9:18068635 |
| 45744 | 9:19093497 | 45768 | 9:19999771 | 45832 | 9:21796887 | 45872 | 9:22692551 | 45957 | 9:24735769 |
| 45958 | 9:24739623 | 45959 | 9:24743503 | 45960 | 9:24744612 | 45961 | 9:24746634 | 45962 | 9:24747990 |
| 45963 | 9:24753059 | 45964 | 9:24757919 | 45965 | 9:24760506 | 45991 | 9:25685943 | 46046 | 9:27095343 |
| 46063 | 9:27473901 | 46065 | 9:27477523 | 46066 | 9:27485623 | 46191 | 9:29796827 | 46248 | 9:31114198 |
| 46259 | 9:31366005 | 46302 | 9:32301933 | 46386 | 9:34555386 | 46398 | 9:34906969 | 46462 | 9:36491907 |
| 46486 | 9:37002303 | 46488 | 9:37019786 | 46516 | 9:37778578 | 46518 | 9:37786775 | 46528 | 9:37990154 |
| 46609 | 9:39610171 | 46636 | 9:39977921 | 46841 | 9:45495778 | 46843 | 9:45524861 | 46886 | 9:46918750 |
| 46890 | 9:47015135 | 46938 | 9:48713344 | 46957 | 9:49281683 | 46958 | 9:49310703 | 46959 | 9:49332688 |
| 46969 | 9:49534025 | 46970 | 9:49560954 | 47019 | 9:50797460 | 47025 | 9:51023481 | 47032 | 9:51178639 |
| 47062 | 9:52118530 | 47097 | 9:52875308 | 47136 | 9:54016591 | 47139 | 9:54071333 | 47141 | 9:54121979 |
| 47143 | 9:54193058 | 47144 | 9:54241895 | 47147 | 9:54281836 | 47156 | 9:54592540 | 47165 | 9:54750120 |
| 47200 | 9:55772680 | 47203 | 9:55920760 | 47212 | 9:56203305 | 47237 | 9:56787230 | 47337 | 9:59589683 |
| 47486 | 9:63468566 | 47687 | 9:67996881 | 47691 | 9:68095521 | 47755 | 9:69703203 | 47782 | 9:70360272 |
| 47785 | 9:70387952 | 47920 | 9:73701336 | 48263 | 9:81076190 | 48268 | 9:81121582 | 48490 | 9:85784712 |
| 48495 | 9:85842378 | 48503 | 9:86006398 | 48511 | 9:86271158 | 48513 | 9:86326905 | 48558 | 9:87361936 |
| 48562 | 9:87394371 | 48620 | 9:88414100 | 48662 | 9:89255756 | 48663 | 9:89261575 | 48664 | 9:89266339 |
| 48691 | 9:89514561 | 48721 | 9:90252969 | 48732 | 9:90513278 | 48805 | 9:92056243 | 48810 | 9:92215180 |
| 48813 | 9:92280929 | 48924 | 9:94493145 | 49009 | 9:96178394 | 49069 | 9:96875482 | 49070 | 9:96875798 |
| 49073 | 9:96892725 | 49074 | 9:96894181 | 49096 | 9:97251687 | 49102 | 9:97327317 | 49126 | 9:97749602 |
| 49382 | 9:102242225 | 49467 | 9:103771813 | 49725 | 10:4803921 | 49789 | 10:5830788 | 49811 | 10:6389416 |
| 49838 | 10:6942796 | 49855 | 10:7506260 | 49870 | 10:7723009 | 49879 | 10:7896282 | 49918 | 10:8581573 |
| 49929 | 10:8932852 | 49954 | 10:9647790 | 49959 | 10:9706130 | 50090 | 10:12326160 | 50252 | 10:15438770 |
| 50302 | 10:16346333 | 50344 | 10:17006334 | 50394 | 10:18062302 | 50406 | 10:18136100 | 50425 | 10:18596826 |
| 50457 | 10:19155115 | 50490 | 10:19862667 | 50499 | 10:19959026 | 50578 | 10:21363314 | 50627 | 10:22355891 |
| 50628 | 10:22382830 | 50704 | 10:26294073 | 50713 | 10:26447113 | 50738 | 10:26790460 | 50793 | 10:27960775 |
| 50811 | 10:28566378 | 50812 | 10:28588817 | 50859 | 10:29085444 | 50868 | 10:29217264 | 50934 | 10:31208483 |
| 50952 | 10:31609187 | 50976 | 10:32324758 | 51059 | 10:34358150 | 51094 | 10:34985737 | 51138 | 10:35989426 |
| 51141 | 10:36035947 | 51147 | 10:36261135 | 51164 | 10:36819725 | 51193 | 10:37476784 | 51242 | 10:38651279 |
| 51294 | 10:40046559 | 51405 | 10:43227119 | 51426 | 10:43996215 | 51527 | 10:46095471 | 51600 | 10:47638179 |
| 51609 | 10:47789244 | 51610 | 10:47842702 | 51626 | 10:48320530 | 51629 | 10:48352886 | 51645 | 10:48589436 |
| 51649 | 10:48670398 | 51770 | 10:51376594 | 51773 | 10:51492328 | 51780 | 10:51664061 | 51801 | 10:52103291 |
| 51809 | 10:52286224 | 51824 | 10:52716176 | 51839 | 10:52867771 | 51896 | 10:54578642 | 51972 | 10:56658480 |
| 52166 | 10:62136409 | 52202 | 10:62942087 | 52272 | 10:64614710 | 52278 | 10:64660791 | 52409 | 10:67916863 |
| 52410 | 10:67917609 | 52411 | 10:67918635 | 52412 | 10:67919519 | 52416 | 10:68020797 | 52439 | 10:68405934 |
| 52471 | 10:69067745 | 52500 | 10:69834381 | 52520 | 10:70404549 | 52616 | 10:73493859 | 52730 | 10:76467129 |
| 52964 | 10:81617198 | 53012 | 10:82478545 | 53038 | 10:83193828 | 53039 | 10:83219578 | 53072 | 10:83953443 |
| 53105 | 10:84613620 | 53132 | 10:85160503 | 53157 | 10:85605717 | 53228 | 10:87305772 | 53332 | 10:89626819 |
| 53351 | 10:89936666 | 53372 | 10:90371369 | 53373 | 10:90396997 | 53380 | 10:90447669 | 53400 | 10:90743393 |
| 53411 | 10:90874981 | 53416 | 10:91003179 | 53424 | 10:91160509 | 53468 | 10:92328861 | 53547 | 10:94201919 |
| 53622 | 10:95898502 | 53688 | 10:97342537 | 53729 | 10:98302579 | 53731 | 10:98340013 | 53748 | 10:98561555 |
| 53769 | 10:99070199 | 53816 | 10:100101830 | 53837 | 10:100508994 | 53879 | 10:101228895 | 53880 | 10:101250880 |
| 53936 | 10:102180727 | 53960 | 10:102751503 | 54049 | 11:1244479 | 54111 | 11:1989902 | 54215 | 11:3345485 |
| 54221 | 11:3483025 | 54298 | 11:5089726 | 54369 | 11:6729148 | 54415 | 11:7795378 | 54432 | 11:8194895 |
| 54454 | 11:9031832 | 54487 | 11:9455596 | 54526 | 11:10334522 | 54533 | 11:10452880 | 54563 | 11:11221986 |
| 54593 | 11:11811943 | 54600 | 11:12001967 | 54611 | 11:12371073 | 54687 | 11:13859460 | 54718 | 11:15010708 |
| 54778 | 11:16210947 | 54799 | 11:16834406 | 54848 | 11:18344213 | 54849 | 11:18370765 | 54850 | 11:18396456 |
| 54959 | 11:21470638 | 55054 | 11:23592030 | 55056 | 11:23656903 | 55060 | 11:23740579 | 55083 | 11:24318038 |
| 55092 | 11:24369984 | 55093 | 11:24377983 | 55094 | 11:24393545 | 55096 | 11:24408850 | 55133 | 11:25341825 |
| 55231 | 11:27746425 | 55274 | 11:28736945 | 55276 | 11:28761952 | 55291 | 11:29024122 | 55369 | 11:30763811 |
| 55384 | 11:31041776 | 55392 | 11:31275179 | 55413 | 11:31882807 | 55416 | 11:31964009 | 55428 | 11:32373197 |
| 55474 | 11:33523211 | 55483 | 11:33718317 | 55559 | 11:36181120 | 55589 | 11:37017087 | 55594 | 11:37101530 |
| 55604 | 11:37283583 | 55776 | 11:41848496 | 55903 | 11:45028224 | 55916 | 11:45290515 | 55917 | 11:45313347 |
| 56006 | 11:47096563 | 56017 | 11:47326731 | 56096 | 11:48675652 | 56133 | 11:49165587 | 56145 | 11:49409414 |
| 56148 | 11:49467653 | 56177 | 11:50202868 | 56186 | 11:50607517 | 56242 | 11:52695227 | 56259 | 11:53270728 |
| 56262 | 11:53413882 | 56431 | 11:57988232 | 56468 | 11:58918358 | 56472 | 11:58971050 | 56480 | 11:59181535 |
| 56524 | 11:60468952 | 56540 | 11:60494769 | 56542 | 11:60517676 | 56544 | 11:60567347 | 56558 | 11:60775322 |
| 56559 | 11:60864552 | 56560 | 11:60906972 | 56594 | 11:61768776 | 56624 | 11:62610532 | 56627 | 11:62639185 |
| 56629 | 11:62703391 | 56635 | 11:62811507 | 56646 | 11:63105834 | 56648 | 11:63156170 | 56688 | 11:63881213 |
| 56694 | 11:63980697 | 56771 | 11:66096418 | 56774 | 11:66141532 | 56792 | 11:66500339 | 56805 | 11:66814255 |
| 56807 | 11:66889704 | 56808 | 11:66971573 | 56813 | 11:67084838 | 56826 | 11:67371438 | 56831 | 11:67445332 |
| 56833 | 11:67469876 | 56877 | 11:67856557 | 56883 | 11:68062927 | 56892 | 11:68380416 | 56894 | 11:68423346 |
| 56911 | 11:68884476 | 56915 | 11:68962608 | 56951 | 11:69554436 | 56976 | 11:70005013 | 56985 | 11:70216257 |
| 56988 | 11:70246998 | 56993 | 11:70300762 | 56996 | 11:70378153 | 57042 | 11:70810858 | 57106 | 11:72079051 |
| 57111 | 11:72169040 | 57219 | 11:74557125 | 57222 | 11:74693404 | 57231 | 11:75039779 | 57238 | 11:75245474 |
| 57239 | 11:75278281 | 57240 | 11:75301517 | 57244 | 11:75330473 | 57261 | 11:75610538 | 57269 | 11:75826371 |
| 57278 | 11:76006290 | 57282 | 11:76108140 | 57286 | 11:76291884 | 57299 | 11:76699098 | 57313 | 11:77009264 |
| 57332 | 11:77262392 | 57343 | 11:77494347 | 57355 | 11:77817486 | 57389 | 11:78010446 | 57391 | 11:78051004 |
| 57414 | 11:78619581 | 57476 | 11:79716380 | 57485 | 11:79945955 | 57486 | 11:80005355 | 57490 | 11:80160161 |
| 57509 | 11:80535106 | 57524 | 11:80835889 | 57534 | 11:81336077 | 57547 | 11:81697133 | 57619 | 11:82922365 |
| 57628 | 11:83118146 | 57667 | 11:84097016 | 57691 | 11:84931500 | 57733 | 11:85771451 | 57735 | 11:85810220 |
| 57756 | 11:86216179 | 57813 | 11:87691339 | 57824 | 11:87901219 | 57825 | 11:87933139 | 57838 | 11:87991576 |
| 57856 | 11:88008802 | 57871 | 11:88092604 | 57875 | 11:88114495 | 57883 | 11:88435971 | 57905 | 11:88999569 |
| 57918 | 11:89363137 | 58014 | 11:91483828 | 58015 | 11:91524490 | 58040 | 11:92016623 | 58199 | 11:95837864 |
| 58223 | 11:96479338 | 58254 | 11:97291509 | 58262 | 11:97559369 | 58265 | 11:97669268 | 58346 | 11:99292200 |
| 58369 | 11:99693004 | 58374 | 11:99779968 | 58381 | 11:99910936 | 58425 | 11:100730926 | 58480 | 11:101720696 |
| 58706 | 11:105729430 | 58710 | 11:105964095 | 58711 | 11:106026712 | 58887 | 12:3816364 | 58893 | 12:4017888 |
| 58924 | 12:4735467 | 59120 | 12:10585260 | 59121 | 12:10614578 | 59249 | 12:13606719 | 59254 | 12:13766685 |
| 59263 | 12:13857247 | 59320 | 12:15004714 | 59351 | 12:15565363 | 59376 | 12:15986894 | 59402 | 12:16562247 |
| 59446 | 12:17721084 | 59635 | 12:22358320 | 59648 | 12:22684761 | 59650 | 12:22780186 | 59656 | 12:22878113 |
| 59657 | 12:22955187 | 59658 | 12:22957971 | 59662 | 12:23024428 | 59686 | 12:23553969 | 59699 | 12:23968918 |
| 59725 | 12:24562389 | 59751 | 12:25043912 | 59818 | 12:26941585 | 59863 | 12:28021962 | 59864 | 12:28031415 |
| 59901 | 12:29149304 | 59931 | 12:30073919 | 59940 | 12:30237550 | 59970 | 12:30965466 | 59975 | 12:30995990 |
| 60105 | 12:34339978 | 60136 | 12:35202030 | 60146 | 12:35329106 | 60185 | 12:36506500 | 60202 | 12:37059151 |
| 60204 | 12:37117228 | 60206 | 12:37121439 | 60213 | 12:37336023 | 60264 | 12:39602051 | 60268 | 12:39726857 |
| 60368 | 12:43543060 | 60451 | 12:46609222 | 60452 | 12:46632594 | 60607 | 12:50328249 | 60608 | 12:50367458 |
| 60610 | 12:50405882 | 60655 | 12:51430846 | 60683 | 12:52226211 | 60688 | 12:52270911 | 60735 | 12:53308347 |
| 60768 | 12:53915464 | 60769 | 12:53924609 | 60780 | 12:54114793 | 60908 | 12:57839576 | 60967 | 12:59688270 |
| 61136 | 12:65124144 | 61209 | 12:67262559 | 61285 | 12:68831445 | 61316 | 12:69427741 | 61337 | 12:69681738 |
| 61357 | 12:70744278 | 61358 | 12:70747193 | 61411 | 12:72948314 | 61418 | 12:73037670 | 61419 | 12:73063509 |
| 61433 | 12:73378861 | 61437 | 12:73452468 | 61748 | 12:80191366 | 61809 | 12:81602505 | 61930 | 12:84189393 |
| 62050 | 12:86604613 | 62158 | 13:1818757 | 62173 | 13:2108292 | 62212 | 13:2694888 | 62217 | 13:2721397 |
| 62219 | 13:2775064 | 62220 | 13:2800282 | 62221 | 13:2813363 | 62257 | 13:3832665 | 62281 | 13:4414592 |
| 62310 | 13:5228105 | 62405 | 13:7418765 | 62519 | 13:10200560 | 62521 | 13:10297467 | 62539 | 13:10605573 |
| 62667 | 13:14736984 | 62723 | 13:16382635 | 62724 | 13:16385352 | 62728 | 13:16481332 | 62729 | 13:16481881 |
| 62730 | 13:16482962 | 62731 | 13:16484064 | 62732 | 13:16486013 | 62733 | 13:16487474 | 62734 | 13:16489553 |
| 62735 | 13:16494712 | 62736 | 13:16495172 | 62737 | 13:16495924 | 62738 | 13:16496828 | 62739 | 13:16497723 |
| 62740 | 13:16498500 | 62741 | 13:16499450 | 62742 | 13:16500106 | 62743 | 13:16505307 | 62774 | 13:17297996 |
| 62785 | 13:17376202 | 62832 | 13:18167455 | 62850 | 13:18708210 | 62851 | 13:18716183 | 62853 | 13:18721208 |
| 62854 | 13:18721251 | 62855 | 13:18723232 | 62856 | 13:18723968 | 62857 | 13:18724391 | 62858 | 13:18724682 |
| 62859 | 13:18724926 | 62860 | 13:18725319 | 62861 | 13:18725595 | 62862 | 13:18725670 | 62863 | 13:18725747 |
| 62864 | 13:18726353 | 62865 | 13:18727332 | 62866 | 13:18727382 | 62867 | 13:18728046 | 62868 | 13:18728216 |
| 62869 | 13:18729395 | 62870 | 13:18729428 | 62871 | 13:18729514 | 62872 | 13:18729597 | 62873 | 13:18732582 |
| 62874 | 13:18733271 | 62875 | 13:18733719 | 62876 | 13:18733742 | 62877 | 13:18733760 | 62878 | 13:18733884 |
| 62879 | 13:18735443 | 62882 | 13:18791943 | 62931 | 13:19690250 | 62932 | 13:19710676 | 62940 | 13:19814205 |
| 62956 | 13:20094078 | 62957 | 13:20180098 | 62960 | 13:20267627 | 62965 | 13:20376037 | 62975 | 13:20455892 |
| 62984 | 13:20631768 | 63067 | 13:22364650 | 63079 | 13:22622712 | 63125 | 13:24378395 | 63206 | 13:26030778 |
| 63286 | 13:28051297 | 63307 | 13:28631602 | 63318 | 13:28789636 | 63452 | 13:31297457 | 63463 | 13:31535167 |
| 63484 | 13:32188070 | 63485 | 13:32192701 | 63486 | 13:32195543 | 63488 | 13:32202239 | 63489 | 13:32206561 |
| 63490 | 13:32214896 | 63498 | 13:32363871 | 63515 | 13:32606184 | 63519 | 13:32649822 | 63534 | 13:32908367 |
| 63643 | 13:35552308 | 63655 | 13:35893139 | 63853 | 13:40115588 | 63937 | 13:42022321 | 63954 | 13:42455044 |
| 63957 | 13:42527459 | 63962 | 13:42563978 | 63996 | 13:42702842 | 64016 | 13:43224017 | 64031 | 13:43618688 |
| 64043 | 13:43868655 | 64084 | 13:44564660 | 64090 | 13:44735441 | 64093 | 13:44846012 | 64094 | 13:44877072 |
| 64095 | 13:44877728 | 64096 | 13:44883203 | 64097 | 13:44884376 | 64098 | 13:44891644 | 64099 | 13:44900803 |
| 64100 | 13:44901430 | 64101 | 13:44902570 | 64102 | 13:44908683 | 64104 | 13:44929091 | 64106 | 13:44941064 |
| 64107 | 13:44945347 | 64108 | 13:44954742 | 64109 | 13:44957480 | 64110 | 13:44959530 | 64111 | 13:44971418 |
| 64113 | 13:44974314 | 64114 | 13:44977218 | 64118 | 13:44984974 | 64120 | 13:44991424 | 64123 | 13:44994329 |
| 64124 | 13:44996032 | 64161 | 13:45580238 | 64220 | 13:46808920 | 64238 | 13:47284141 | 64266 | 13:47867949 |
| 64336 | 13:49568372 | 64337 | 13:49569350 | 64338 | 13:49570779 | 64339 | 13:49571658 | 64416 | 13:52005450 |
| 64426 | 13:52366895 | 64431 | 13:52518024 | 64456 | 13:52984857 | 64458 | 13:53006663 | 64466 | 13:53163449 |
| 64512 | 13:54065681 | 64629 | 13:57014498 | 64667 | 13:57988686 | 64672 | 13:58092691 | 64887 | 13:62737371 |
| 64900 | 13:62849693 | 64901 | 13:62871349 | 64910 | 13:63113668 | 64979 | 13:64765574 | 64992 | 13:65197131 |
| 65003 | 13:65421742 | 65046 | 13:66169238 | 65096 | 13:67321578 | 65143 | 13:68639312 | 65145 | 13:68664064 |
| 65159 | 13:69019407 | 65312 | 13:72652771 | 65332 | 13:73024654 | 65430 | 13:74713520 | 65439 | 13:75021624 |
| 65447 | 13:75318964 | 65448 | 13:75354084 | 65503 | 13:76376258 | 65507 | 13:76438492 | 65517 | 13:76641970 |
| 65529 | 13:76798256 | 65764 | 13:81126899 | 65905 | 14:1006940 | 65963 | 14:2212373 | 65993 | 14:3074708 |
| 65995 | 14:3098041 | 66091 | 14:4323988 | 66140 | 14:5386856 | 66177 | 14:5966351 | 66207 | 14:6303201 |
| 66289 | 14:7818027 | 66305 | 14:8003665 | 66326 | 14:8349092 | 66332 | 14:8441986 | 66350 | 14:8630299 |
| 66373 | 14:8996524 | 66493 | 14:11272085 | 66494 | 14:11275856 | 66495 | 14:11277881 | 66496 | 14:11278042 |
| 66497 | 14:11281126 | 66499 | 14:11294774 | 66500 | 14:11299050 | 66547 | 14:12222307 | 66586 | 14:12811226 |
| 66627 | 14:14043986 | 66687 | 14:15518676 | 66690 | 14:15530827 | 66691 | 14:15559743 | 66692 | 14:15601008 |
| 66693 | 14:15625272 | 66694 | 14:15626105 | 66695 | 14:15631968 | 66696 | 14:15643325 | 66698 | 14:15672366 |
| 66717 | 14:16009930 | 66718 | 14:16010978 | 66719 | 14:16022809 | 66724 | 14:16161546 | 66750 | 14:16670887 |
| 66775 | 14:17233811 | 66795 | 14:17760515 | 66799 | 14:17770830 | 66831 | 14:18480356 | 66832 | 14:18510376 |
| 66909 | 14:20574088 | 66969 | 14:21764414 | 66976 | 14:21914329 | 66987 | 14:22199112 | 66989 | 14:22291692 |
| 66991 | 14:22353534 | 66994 | 14:22402037 | 66997 | 14:22459009 | 66998 | 14:22460066 | 66999 | 14:22492158 |
| 67000 | 14:22499302 | 67001 | 14:22499882 | 67002 | 14:22500823 | 67003 | 14:22500981 | 67004 | 14:22501165 |
| 67005 | 14:22502609 | 67006 | 14:22503786 | 67007 | 14:22505668 | 67009 | 14:22551848 | 67010 | 14:22559672 |
| 67011 | 14:22559976 | 67012 | 14:22560498 | 67013 | 14:22560939 | 67014 | 14:22561597 | 67015 | 14:22562389 |
| 67016 | 14:22562808 | 67017 | 14:22562827 | 67018 | 14:22563098 | 67019 | 14:22563125 | 67020 | 14:22563136 |
| 67021 | 14:22563534 | 67022 | 14:22564168 | 67023 | 14:22564379 | 67024 | 14:22564874 | 67025 | 14:22565102 |
| 67026 | 14:22565724 | 67027 | 14:22565752 | 67028 | 14:22565896 | 67029 | 14:22565903 | 67030 | 14:22566273 |
| 67031 | 14:22566433 | 67032 | 14:22566621 | 67033 | 14:22567218 | 67034 | 14:22567252 | 67035 | 14:22567396 |
| 67036 | 14:22567418 | 67037 | 14:22567497 | 67038 | 14:22567570 | 67039 | 14:22568006 | 67040 | 14:22568446 |
| 67041 | 14:22568460 | 67042 | 14:22568517 | 67043 | 14:22568557 | 67044 | 14:22568700 | 67045 | 14:22568919 |
| 67046 | 14:22569314 | 67047 | 14:22569464 | 67048 | 14:22569577 | 67049 | 14:22569581 | 67050 | 14:22569980 |
| 67051 | 14:22570044 | 67052 | 14:22570058 | 67053 | 14:22570080 | 67054 | 14:22570301 | 67055 | 14:22570448 |
| 67056 | 14:22570466 | 67057 | 14:22570726 | 67058 | 14:22571422 | 67059 | 14:22573154 | 67060 | 14:22574799 |
| 67061 | 14:22579399 | 67062 | 14:22579809 | 67063 | 14:22581401 | 67064 | 14:22584062 | 67065 | 14:22584452 |
| 67066 | 14:22592312 | 67071 | 14:22671158 | 67072 | 14:22680795 | 67073 | 14:22681826 | 67079 | 14:22781305 |
| 67080 | 14:22830132 | 67081 | 14:22839488 | 67082 | 14:22841040 | 67083 | 14:22842180 | 67084 | 14:22861656 |
| 67085 | 14:22861757 | 67086 | 14:22861840 | 67087 | 14:22866278 | 67088 | 14:22867321 | 67089 | 14:22870443 |
| 67090 | 14:22872231 | 67091 | 14:22872707 | 67092 | 14:22875389 | 67094 | 14:22883080 | 67096 | 14:22887406 |
| 67097 | 14:22888105 | 67098 | 14:22895284 | 67099 | 14:22896212 | 67100 | 14:22899088 | 67101 | 14:22900130 |
| 67102 | 14:22900140 | 67103 | 14:22903945 | 67104 | 14:22904357 | 67105 | 14:22905670 | 67106 | 14:22915081 |
| 67107 | 14:22915304 | 67108 | 14:22916046 | 67109 | 14:22916365 | 67110 | 14:22917130 | 67111 | 14:22917282 |
| 67112 | 14:22918679 | 67113 | 14:22919099 | 67114 | 14:22922271 | 67115 | 14:22925305 | 67116 | 14:22926221 |
| 67117 | 14:22933624 | 67118 | 14:22935277 | 67119 | 14:22940329 | 67120 | 14:22941327 | 67121 | 14:22944551 |
| 67122 | 14:22945851 | 67123 | 14:22950339 | 67124 | 14:22951594 | 67125 | 14:22952187 | 67126 | 14:22952264 |
| 67127 | 14:22958318 | 67128 | 14:22959278 | 67129 | 14:22962984 | 67130 | 14:22963530 | 67131 | 14:22963552 |
| 67132 | 14:22963956 | 67133 | 14:22964530 | 67134 | 14:22968925 | 67135 | 14:22969225 | 67136 | 14:22969659 |
| 67137 | 14:22970008 | 67138 | 14:22983250 | 67139 | 14:22983836 | 67140 | 14:22986080 | 67141 | 14:22989108 |
| 67142 | 14:22989880 | 67143 | 14:22989917 | 67144 | 14:22995750 | 67145 | 14:22995861 | 67146 | 14:22999212 |
| 67147 | 14:22999475 | 67148 | 14:23000859 | 67149 | 14:23001109 | 67150 | 14:23001803 | 67151 | 14:23004072 |
| 67152 | 14:23005237 | 67153 | 14:23009266 | 67157 | 14:23168386 | 67175 | 14:23216693 | 67180 | 14:23217714 |
| 67181 | 14:23217718 | 67183 | 14:23219140 | 67189 | 14:23221714 | 67191 | 14:23232498 | 67193 | 14:23241336 |
| 67194 | 14:23244133 | 67195 | 14:23245996 | 67196 | 14:23246004 | 67197 | 14:23246030 | 67198 | 14:23246116 |
| 67199 | 14:23246121 | 67200 | 14:23246668 | 67201 | 14:23247018 | 67202 | 14:23247588 | 67203 | 14:23247661 |
| 67204 | 14:23247870 | 67205 | 14:23247878 | 67206 | 14:23247920 | 67207 | 14:23247940 | 67208 | 14:23248135 |
| 67209 | 14:23248201 | 67210 | 14:23248211 | 67211 | 14:23248448 | 67212 | 14:23248692 | 67213 | 14:23248770 |
| 67214 | 14:23248888 | 67215 | 14:23248943 | 67216 | 14:23249702 | 67217 | 14:23249738 | 67218 | 14:23249950 |
| 67219 | 14:23249976 | 67220 | 14:23250082 | 67221 | 14:23250315 | 67222 | 14:23250686 | 67223 | 14:23250690 |
| 67224 | 14:23250828 | 67225 | 14:23250871 | 67226 | 14:23250914 | 67227 | 14:23251020 | 67228 | 14:23251302 |
| 67229 | 14:23251581 | 67230 | 14:23251684 | 67231 | 14:23252354 | 67232 | 14:23252429 | 67233 | 14:23252563 |
| 67234 | 14:23252757 | 67235 | 14:23252809 | 67236 | 14:23253043 | 67237 | 14:23253210 | 67238 | 14:23253295 |
| 67239 | 14:23253300 | 67240 | 14:23253568 | 67241 | 14:23253746 | 67242 | 14:23253786 | 67243 | 14:23253805 |
| 67244 | 14:23254426 | 67245 | 14:23254453 | 67246 | 14:23254563 | 67247 | 14:23254639 | 67248 | 14:23254769 |
| 67249 | 14:23254807 | 67250 | 14:23254919 | 67251 | 14:23255007 | 67252 | 14:23255159 | 67253 | 14:23255587 |
| 67254 | 14:23255824 | 67255 | 14:23256710 | 67256 | 14:23257050 | 67257 | 14:23257092 | 67258 | 14:23257183 |
| 67259 | 14:23257229 | 67260 | 14:23257379 | 67261 | 14:23257441 | 67262 | 14:23257644 | 67263 | 14:23257665 |
| 67264 | 14:23257775 | 67265 | 14:23258452 | 67266 | 14:23258542 | 67267 | 14:23262536 | 67268 | 14:23262544 |
| 67269 | 14:23262984 | 67270 | 14:23264575 | 67271 | 14:23264736 | 67272 | 14:23265786 | 67273 | 14:23265991 |
| 67274 | 14:23266384 | 67275 | 14:23271813 | 67276 | 14:23273276 | 67277 | 14:23273801 | 67278 | 14:23274957 |
| 67279 | 14:23277049 | 67280 | 14:23278232 | 67281 | 14:23278652 | 67282 | 14:23278927 | 67283 | 14:23279593 |
| 67284 | 14:23279712 | 67285 | 14:23285130 | 67286 | 14:23288477 | 67287 | 14:23288921 | 67288 | 14:23289158 |
| 67289 | 14:23289589 | 67290 | 14:23289947 | 67291 | 14:23292612 | 67293 | 14:23296927 | 67294 | 14:23297204 |
| 67295 | 14:23298062 | 67296 | 14:23300304 | 67297 | 14:23314460 | 67298 | 14:23314730 | 67299 | 14:23314761 |
| 67300 | 14:23321577 | 67301 | 14:23326588 | 67302 | 14:23338890 | 67303 | 14:23343150 | 67304 | 14:23346065 |
| 67306 | 14:23383008 | 67308 | 14:23392938 | 67309 | 14:23408928 | 67310 | 14:23409204 | 67311 | 14:23409494 |
| 67312 | 14:23410644 | 67313 | 14:23410867 | 67314 | 14:23411712 | 67315 | 14:23414348 | 67316 | 14:23414443 |
| 67317 | 14:23421256 | 67318 | 14:23421807 | 67319 | 14:23422909 | 67320 | 14:23423768 | 67321 | 14:23423823 |
| 67322 | 14:23424726 | 67323 | 14:23426056 | 67324 | 14:23427548 | 67325 | 14:23429529 | 67326 | 14:23430628 |
| 67327 | 14:23430999 | 67328 | 14:23431167 | 67329 | 14:23438214 | 67330 | 14:23439941 | 67331 | 14:23440995 |
| 67332 | 14:23441483 | 67333 | 14:23443135 | 67334 | 14:23444334 | 67335 | 14:23446328 | 67336 | 14:23447775 |
| 67337 | 14:23450091 | 67338 | 14:23456836 | 67339 | 14:23458201 | 67340 | 14:23459603 | 67343 | 14:23528515 |
| 67344 | 14:23532117 | 67354 | 14:23600267 | 67355 | 14:23607944 | 67356 | 14:23622754 | 67357 | 14:23630896 |
| 67358 | 14:23639458 | 67359 | 14:23652804 | 67368 | 14:23825669 | 67370 | 14:23829057 | 67371 | 14:23829066 |
| 67372 | 14:23829235 | 67373 | 14:23830314 | 67375 | 14:23831223 | 67376 | 14:23831351 | 67377 | 14:23831451 |
| 67378 | 14:23831720 | 67379 | 14:23832062 | 67380 | 14:23832448 | 67381 | 14:23832632 | 67382 | 14:23832846 |
| 67383 | 14:23832941 | 67384 | 14:23833173 | 67385 | 14:23833835 | 67386 | 14:23834262 | 67387 | 14:23834735 |
| 67388 | 14:23834925 | 67389 | 14:23835136 | 67390 | 14:23835627 | 67391 | 14:23835870 | 67392 | 14:23835971 |
| 67393 | 14:23836131 | 67394 | 14:23836398 | 67395 | 14:23836443 | 67396 | 14:23836603 | 67397 | 14:23836691 |
| 67398 | 14:23836914 | 67399 | 14:23837002 | 67400 | 14:23838557 | 67401 | 14:23839177 | 67402 | 14:23840827 |
| 67403 | 14:23842863 | 67404 | 14:23848780 | 67405 | 14:23848797 | 67406 | 14:23848827 | 67407 | 14:23849503 |
| 67408 | 14:23852492 | 67409 | 14:23852815 | 67410 | 14:23852921 | 67411 | 14:23853184 | 67412 | 14:23853223 |
| 67413 | 14:23854487 | 67414 | 14:23856672 | 67415 | 14:23860303 | 67416 | 14:23862417 | 67418 | 14:23868149 |
| 67419 | 14:23882209 | 67420 | 14:23887365 | 67421 | 14:23887370 | 67422 | 14:23937950 | 67423 | 14:23945118 |
| 67425 | 14:23992830 | 67426 | 14:24019648 | 67430 | 14:24107226 | 67436 | 14:24297582 | 67440 | 14:24350585 |
| 67446 | 14:24477168 | 67447 | 14:24512384 | 67449 | 14:24516122 | 67450 | 14:24519148 | 67451 | 14:24519442 |
| 67452 | 14:24520182 | 67453 | 14:24522732 | 67454 | 14:24524138 | 67455 | 14:24527219 | 67456 | 14:24528754 |
| 67457 | 14:24532678 | 67458 | 14:24535392 | 67459 | 14:24536887 | 67460 | 14:24537213 | 67461 | 14:24537249 |
| 67462 | 14:24537652 | 67463 | 14:24537937 | 67464 | 14:24538192 | 67465 | 14:24538201 | 67466 | 14:24538525 |
| 67467 | 14:24538984 | 67468 | 14:24539180 | 67469 | 14:24539228 | 67470 | 14:24539315 | 67471 | 14:24539324 |
| 67472 | 14:24539556 | 67473 | 14:24539584 | 67474 | 14:24539696 | 67475 | 14:24540007 | 67476 | 14:24540057 |
| 67477 | 14:24540244 | 67478 | 14:24540270 | 67479 | 14:24542751 | 67481 | 14:24571024 | 67482 | 14:24580144 |
| 67483 | 14:24587368 | 67484 | 14:24594780 | 67485 | 14:24604764 | 67486 | 14:24609404 | 67487 | 14:24618912 |
| 67488 | 14:24644457 | 67493 | 14:24767026 | 67495 | 14:24790463 | 67496 | 14:24825146 | 67497 | 14:24859655 |
| 67500 | 14:24940746 | 67501 | 14:24941221 | 67502 | 14:24967239 | 67503 | 14:24967988 | 67504 | 14:24973247 |
| 67505 | 14:24978104 | 67506 | 14:24979482 | 67507 | 14:24982302 | 67508 | 14:24983741 | 67509 | 14:24986291 |
| 67510 | 14:25002052 | 67511 | 14:25030583 | 67513 | 14:25082860 | 67516 | 14:25152867 | 67517 | 14:25158355 |
| 67526 | 14:25266063 | 67530 | 14:25440652 | 67533 | 14:25548588 | 67534 | 14:25548611 | 67535 | 14:25548858 |
| 67536 | 14:25549944 | 67537 | 14:25550026 | 67538 | 14:25550835 | 67539 | 14:25550879 | 67540 | 14:25551206 |
| 67541 | 14:25551423 | 67542 | 14:25551661 | 67543 | 14:25551868 | 67544 | 14:25552256 | 67545 | 14:25552486 |
| 67546 | 14:25552782 | 67547 | 14:25552984 | 67548 | 14:25553034 | 67549 | 14:25553066 | 67550 | 14:25553217 |
| 67552 | 14:25555831 | 67613 | 14:26283998 | 67621 | 14:26472341 | 67627 | 14:26644064 | 67628 | 14:26666519 |
| 67686 | 14:28503857 | 67690 | 14:28611968 | 67696 | 14:28706164 | 67714 | 14:29246244 | 67717 | 14:29321450 |
| 67723 | 14:29455092 | 67743 | 14:29818291 | 67756 | 14:30089537 | 67838 | 14:32001174 | 67864 | 14:32341495 |
| 67882 | 14:32807225 | 67883 | 14:32884654 | 67887 | 14:32989933 | 67910 | 14:33408077 | 67918 | 14:33505061 |
| 67920 | 14:33551756 | 67973 | 14:34950627 | 67979 | 14:35239973 | 67980 | 14:35240016 | 67981 | 14:35240021 |
| 67982 | 14:35240029 | 67983 | 14:35240427 | 67984 | 14:35240523 | 67985 | 14:35240587 | 67986 | 14:35240759 |
| 67987 | 14:35240947 | 67988 | 14:35241115 | 67989 | 14:35241328 | 67990 | 14:35241474 | 67991 | 14:35241672 |
| 67992 | 14:35265340 | 67998 | 14:35425257 | 68003 | 14:35595531 | 68006 | 14:35751572 | 68010 | 14:35894072 |
| 68024 | 14:36171691 | 68025 | 14:36225604 | 68027 | 14:36267667 | 68045 | 14:36581802 | 68070 | 14:37486005 |
| 68085 | 14:37852302 | 68092 | 14:38020858 | 68098 | 14:38241778 | 68104 | 14:38511542 | 68107 | 14:38549650 |
| 68121 | 14:38865578 | 68136 | 14:39381019 | 68138 | 14:39446327 | 68154 | 14:39861013 | 68181 | 14:40565849 |
| 68184 | 14:40702265 | 68188 | 14:40809782 | 68195 | 14:41149021 | 68202 | 14:41340996 | 68219 | 14:41739836 |
| 68239 | 14:42191184 | 68242 | 14:42286395 | 68265 | 14:42846993 | 68302 | 14:44079419 | 68338 | 14:44933472 |
| 68350 | 14:45112392 | 68358 | 14:45283009 | 68368 | 14:45610432 | 68374 | 14:45681264 | 68376 | 14:45772293 |
| 68391 | 14:46030748 | 68402 | 14:46236143 | 68403 | 14:46236637 | 68404 | 14:46237110 | 68405 | 14:46238761 |
| 68406 | 14:46241868 | 68407 | 14:46244088 | 68408 | 14:46250918 | 68418 | 14:46418593 | 68424 | 14:46603107 |
| 68441 | 14:46995812 | 68444 | 14:47059409 | 68474 | 14:48033192 | 68483 | 14:48202902 | 68512 | 14:48835114 |
| 68819 | 14:56925358 | 68973 | 14:60573159 | 68974 | 14:60591022 | 69042 | 14:62002265 | 69055 | 14:62159723 |
| 69056 | 14:62160097 | 69093 | 14:62960532 | 69145 | 14:64219535 | 69158 | 14:64538337 | 69236 | 14:66565905 |
| 69296 | 14:67751963 | 69405 | 14:70557126 | 69479 | 14:72621724 | 69527 | 14:73767156 | 69546 | 14:74251050 |
| 69666 | 14:77010786 | 69676 | 14:77348388 | 69701 | 14:77989698 | 69824 | 14:80695704 | 69841 | 14:81015684 |
| 70102 | 15:4692832 | 70287 | 15:5550484 | 70295 | 15:5724091 | 70403 | 15:8239471 | 70559 | 15:13400749 |
| 70578 | 15:14002939 | 70584 | 15:14158577 | 70619 | 15:14996109 | 70634 | 15:15286305 | 70711 | 15:16549253 |
| 70719 | 15:16763241 | 70746 | 15:17193061 | 70781 | 15:18160360 | 70783 | 15:18238844 | 70920 | 15:21418237 |
| 70933 | 15:21792771 | 70955 | 15:22295223 | 71033 | 15:23794449 | 71075 | 15:24300165 | 71076 | 15:24314377 |
| 71084 | 15:24406959 | 71092 | 15:24625723 | 71106 | 15:24813538 | 71225 | 15:27497541 | 71246 | 15:28011102 |
| 71266 | 15:28279946 | 71274 | 15:28453282 | 71275 | 15:28497953 | 71313 | 15:28951920 | 71373 | 15:30506918 |
| 71421 | 15:31626657 | 71443 | 15:32064162 | 71459 | 15:32448502 | 71467 | 15:32684505 | 71489 | 15:33066384 |
| 71494 | 15:33140066 | 71500 | 15:33283643 | 71516 | 15:33757310 | 71519 | 15:33784264 | 71542 | 15:34144843 |
| 71544 | 15:34205212 | 71548 | 15:34249244 | 71550 | 15:34273013 | 71565 | 15:34545054 | 71594 | 15:35124174 |
| 71637 | 15:36171696 | 71643 | 15:36281231 | 71644 | 15:36316689 | 71668 | 15:36835278 | 71670 | 15:36913421 |
| 71721 | 15:38013431 | 71735 | 15:38345972 | 71814 | 15:40225597 | 71892 | 15:41883212 | 71918 | 15:42285508 |
| 71919 | 15:42310362 | 71922 | 15:42461633 | 71961 | 15:43061218 | 71966 | 15:43113772 | 71983 | 15:43322372 |
| 72013 | 15:43808690 | 72034 | 15:44213989 | 72038 | 15:44321644 | 72039 | 15:44348210 | 72041 | 15:44377304 |
| 72043 | 15:44404296 | 72081 | 15:45199864 | 72107 | 15:45767348 | 72150 | 15:46515608 | 72163 | 15:46717734 |
| 72166 | 15:46782723 | 72172 | 15:46863275 | 72181 | 15:47142381 | 72226 | 15:48040954 | 72245 | 15:48275779 |
| 72278 | 15:49421011 | 72280 | 15:49469161 | 72294 | 15:49994423 | 72324 | 15:51010384 | 72368 | 15:51564179 |
| 72387 | 15:51844292 | 72392 | 15:51981969 | 72395 | 15:51992306 | 72396 | 15:52000306 | 72403 | 15:52160095 |
| 72434 | 15:52969357 | 72473 | 15:53341502 | 72559 | 15:55149996 | 72592 | 15:56124060 | 72635 | 15:56767431 |
| 72636 | 15:56774921 | 72637 | 15:56780183 | 72638 | 15:56782530 | 72639 | 15:56783614 | 72640 | 15:56784736 |
| 72641 | 15:56785971 | 72642 | 15:56797332 | 72643 | 15:56807110 | 72646 | 15:56815479 | 72647 | 15:56818346 |
| 72648 | 15:56829119 | 72649 | 15:56831561 | 72650 | 15:56832894 | 72651 | 15:56833644 | 72652 | 15:56834347 |
| 72653 | 15:56835434 | 72654 | 15:56836055 | 72655 | 15:56836949 | 72656 | 15:56838230 | 72657 | 15:56838871 |
| 72658 | 15:56840204 | 72659 | 15:56841525 | 72660 | 15:56842815 | 72661 | 15:56843668 | 72662 | 15:56845155 |
| 72663 | 15:56851874 | 72664 | 15:56853748 | 72665 | 15:56855128 | 72666 | 15:56856657 | 72667 | 15:56868653 |
| 72668 | 15:56869977 | 72690 | 15:57484879 | 72695 | 15:57536250 | 72767 | 15:58882110 | 72769 | 15:58979088 |
| 72784 | 15:59472006 | 72833 | 15:60847989 | 72869 | 15:61639615 | 72884 | 15:61810673 | 72900 | 15:62235431 |
| 72906 | 15:62456943 | 72909 | 15:62484563 | 72910 | 15:62511268 | 72918 | 15:62618258 | 72922 | 15:62694367 |
| 72976 | 15:63833091 | 72988 | 15:64092090 | 73022 | 15:64696473 | 73048 | 15:65009502 | 73052 | 15:65067229 |
| 73130 | 15:66545124 | 73132 | 15:66614036 | 73460 | 15:75001650 | 73465 | 15:75074384 | 73560 | 15:77284842 |
| 73564 | 15:77342823 | 73599 | 15:77992295 | 73602 | 15:78001169 | 73610 | 15:78017138 | 73626 | 15:78135787 |
| 73719 | 15:80696386 | 73731 | 15:81021484 | 73760 | 15:81377169 | 73768 | 15:81639702 | 73794 | 15:82390046 |
| 73813 | 15:82751747 | 73814 | 15:82766106 | 73826 | 15:82951372 | 73834 | 15:83031817 | 73835 | 15:83043883 |
| 73836 | 15:83046338 | 73837 | 15:83054032 | 73838 | 15:83058015 | 73839 | 15:83063624 | 73945 | 16:1306469 |
| 73949 | 16:1350556 | 73955 | 16:1432322 | 74009 | 16:2521425 | 74012 | 16:2584565 | 74027 | 16:3043683 |
| 74058 | 16:3453206 | 74076 | 16:3954831 | 74079 | 16:3986796 | 74218 | 16:7674017 | 74248 | 16:8845857 |
| 74278 | 16:9938750 | 74286 | 16:10168137 | 74356 | 16:12347316 | 74366 | 16:12489625 | 74382 | 16:12870890 |
| 74389 | 16:12974308 | 74390 | 16:12974917 | 74391 | 16:12975122 | 74392 | 16:12975220 | 74393 | 16:12975427 |
| 74394 | 16:12975489 | 74395 | 16:12975573 | 74396 | 16:12975644 | 74460 | 16:14726498 | 74462 | 16:14841844 |
| 74520 | 16:16433701 | 74547 | 16:17397768 | 74579 | 16:18551432 | 74583 | 16:18633047 | 74592 | 16:18835344 |
| 74621 | 16:19568001 | 74641 | 16:20043212 | 74669 | 16:20811063 | 74702 | 16:21584184 | 74853 | 16:25049887 |
| 74963 | 16:26927766 | 74986 | 16:27682201 | 75041 | 16:29174251 | 75060 | 16:29760720 | 75062 | 16:29811081 |
| 75073 | 16:30136403 | 75075 | 16:30161226 | 75076 | 16:30185523 | 75134 | 16:31704359 | 75143 | 16:31896105 |
| 75150 | 16:32035645 | 75163 | 16:32472255 | 75166 | 16:32570779 | 75268 | 16:35045999 | 75271 | 16:35171790 |
| 75403 | 16:38151804 | 75444 | 16:39447477 | 75514 | 16:41852637 | 75544 | 16:41964938 | 75601 | 16:44301023 |
| 75602 | 16:44309062 | 75603 | 16:44341029 | 75675 | 16:46932908 | 75678 | 16:47002315 | 75805 | 16:49746045 |
| 75821 | 16:50190328 | 75822 | 16:50240719 | 75839 | 16:50745473 | 75941 | 16:53280547 | 75975 | 16:54055090 |
| 75976 | 16:54077469 | 75987 | 16:54386293 | 76037 | 16:55777077 | 76379 | 16:63448062 | 76420 | 16:64372019 |
| 76474 | 16:65163914 | 76705 | 16:70129856 | 76767 | 16:71527211 | 76903 | 16:74041271 | 76913 | 16:74276107 |
| 76922 | 16:74520515 | 76932 | 16:74777059 | 76992 | 16:75746134 | 77018 | 16:76272938 | 77019 | 16:76273016 |
| 77054 | 16:77113967 | 77055 | 16:77114869 | 77056 | 16:77115406 | 77057 | 16:77115489 | 77058 | 16:77115511 |
| 77059 | 16:77115516 | 77060 | 16:77117453 | 77061 | 16:77124198 | 77227 | 16:80002018 | 77342 | 17:2538151 |
| 77417 | 17:4163909 | 77468 | 17:5077558 | 77480 | 17:5375823 | 77508 | 17:5999177 | 77613 | 17:8012177 |
| 77632 | 17:8453908 | 77860 | 17:14044398 | 77915 | 17:14966600 | 78029 | 17:17475831 | 78037 | 17:17737256 |
| 78038 | 17:17740826 | 78097 | 17:19033632 | 78335 | 17:26269335 | 78410 | 17:28015501 | 78497 | 17:30487913 |
| 78560 | 17:31986086 | 78574 | 17:32403842 | 78580 | 17:32489547 | 78638 | 17:33714773 | 78647 | 17:33972684 |
| 78649 | 17:34035563 | 78655 | 17:34228544 | 78680 | 17:34921588 | 78708 | 17:35827456 | 78896 | 17:40827993 |
| 79097 | 17:45794152 | 79104 | 17:45901126 | 79186 | 17:47856402 | 79401 | 17:52535678 | 79405 | 17:52588133 |
| 79527 | 17:55767778 | 79528 | 17:55769246 | 79534 | 17:55892881 | 79577 | 17:57167393 | 79582 | 17:57279391 |
| 79748 | 17:60357076 | 79754 | 17:60395994 | 79778 | 17:60781502 | 79835 | 17:61664121 | 79837 | 17:61736121 |
| 79860 | 17:62385783 | 79871 | 17:62699773 | 79873 | 17:62731132 | 79876 | 17:62794522 | 79877 | 17:62820495 |
| 79916 | 17:63612425 | 79928 | 17:63638557 | 79962 | 17:64318575 | 80008 | 17:65293792 | 80030 | 17:65746912 |
| 80050 | 17:66155014 | 80061 | 17:66243332 | 80076 | 17:66473136 | 80079 | 17:66581559 | 80083 | 17:66695254 |
| 80109 | 17:67239268 | 80249 | 17:70356568 | 80285 | 17:70987613 | 80303 | 17:71333851 | 80363 | 17:72400135 |
| 80592 | 18:3769740 | 80636 | 18:4682446 | 80731 | 18:6411919 | 80773 | 18:7301014 | 80800 | 18:7879780 |
| 80807 | 18:8137616 | 80811 | 18:8163590 | 80812 | 18:8166965 | 80813 | 18:8169505 | 80848 | 18:8910517 |
| 80881 | 18:9393342 | 80887 | 18:9441291 | 80933 | 18:10341158 | 80973 | 18:11082461 | 81013 | 18:11950846 |
| 81055 | 18:13195401 | 81339 | 18:19844264 | 81450 | 18:22200958 | 81455 | 18:22265614 | 81495 | 18:23178646 |
| 81501 | 18:23288724 | 81645 | 18:26295597 | 81707 | 18:27873728 | 81901 | 18:33007379 | 81923 | 18:33448672 |
| 81932 | 18:33625498 | 81933 | 18:33654608 | 81936 | 18:33679712 | 81937 | 18:33689480 | 81939 | 18:33731026 |
| 81940 | 18:33765822 | 81945 | 18:33836053 | 81946 | 18:33868226 | 82061 | 18:36768945 | 82065 | 18:36877351 |
| 82083 | 18:37328492 | 82099 | 18:37566458 | 82114 | 18:37873077 | 82177 | 18:39306524 | 82178 | 18:39344592 |
| 82179 | 18:39385561 | 82181 | 18:39440689 | 82184 | 18:39467461 | 82186 | 18:39488321 | 82190 | 18:39592965 |
| 82370 | 18:43157279 | 82379 | 18:43316323 | 82382 | 18:43368967 | 82508 | 18:46424023 | 82580 | 18:46784427 |
| 82583 | 18:46865748 | 82670 | 18:48351248 | 82698 | 18:49004338 | 82732 | 18:49949153 | 82736 | 18:49996660 |
| 82831 | 18:52094939 | 82855 | 18:52727408 | 82885 | 18:53596284 | 82887 | 18:53619711 | 82909 | 18:53938427 |
| 82919 | 18:54093736 | 82940 | 18:54546398 | 82942 | 18:54586408 | 82983 | 18:55141854 | 82984 | 18:55173246 |
| 83013 | 18:55944026 | 83110 | 18:57018026 | 83153 | 18:57798250 | 83172 | 18:57998293 | 83257 | 18:59897080 |
| 83291 | 18:61008032 | 83319 | 18:61724391 | 83595 | 19:414615 | 84130 | 19:13223983 | 84131 | 19:13225470 |
| 84175 | 19:13777565 | 84315 | 19:16698614 | 84335 | 19:16990785 | 84387 | 19:17914841 | 84393 | 19:18036338 |
| 84409 | 19:18449930 | 84457 | 19:19539554 | 84577 | 19:21388744 | 84702 | 19:23904908 | 84703 | 19:23933777 |
| 84742 | 19:24383348 | 84781 | 19:24846129 | 84846 | 19:25706233 | 85007 | 19:28109982 | 85011 | 19:28147842 |
| 85061 | 19:29186854 | 85119 | 19:30424898 | 85120 | 19:30433795 | 85121 | 19:30435544 | 85178 | 19:31844296 |
| 85211 | 19:32510963 | 85345 | 19:34890238 | 85357 | 19:34981210 | 85366 | 19:35152019 | 85409 | 19:36144917 |
| 85481 | 19:37101024 | 85602 | 19:40338519 | 85855 | 19:45407828 | 85921 | 19:46516531 | 85941 | 19:47101703 |
| 85951 | 19:47151917 | 85971 | 19:47631810 | 86015 | 19:48407091 | 86087 | 19:49926869 | 86124 | 19:50694652 |
| 86143 | 19:50789408 | 86202 | 19:51325894 | 86259 | 19:52650212 | 86283 | 19:53097970 | 86292 | 19:53198253 |
| 86293 | 19:53206247 | 86319 | 19:53762930 | 86348 | 19:54137367 | 86384 | 19:54758347 | 86416 | 19:55474861 |
| 86488 | 19:56006886 | 86522 | 19:56138971 | 86531 | 19:56253321 | 86568 | 19:56726108 | 86580 | 19:56980273 |
| 86813 | 19:61050416 | 86822 | 19:61175458 | 86893 | 19:62160575 | 86898 | 19:62249612 | 86900 | 19:62272214 |
| 86997 | 20:909954 | 87043 | 20:1738655 | 87052 | 20:1886807 | 87083 | 20:2362867 | 87101 | 20:2682912 |
| 87142 | 20:3442790 | 87149 | 20:3585646 | 87174 | 20:4263007 | 87176 | 20:4284310 | 87177 | 20:4331799 |
| 87190 | 20:4516902 | 87193 | 20:4587717 | 87194 | 20:4587833 | 87195 | 20:4587839 | 87196 | 20:4588506 |
| 87197 | 20:4588549 | 87198 | 20:4588618 | 87199 | 20:4588637 | 87200 | 20:4588654 | 87203 | 20:4620871 |
| 87204 | 20:4621075 | 87205 | 20:4621110 | 87206 | 20:4621122 | 87207 | 20:4622072 | 87208 | 20:4622166 |
| 87209 | 20:4622378 | 87210 | 20:4622780 | 87211 | 20:4622804 | 87212 | 20:4622902 | 87213 | 20:4623044 |
| 87214 | 20:4623114 | 87215 | 20:4623572 | 87216 | 20:4624342 | 87217 | 20:4624492 | 87218 | 20:4624645 |
| 87219 | 20:4625064 | 87220 | 20:4625438 | 87221 | 20:4626150 | 87222 | 20:4626194 | 87223 | 20:4626939 |
| 87224 | 20:4627062 | 87225 | 20:4628179 | 87226 | 20:4628239 | 87227 | 20:4629196 | 87228 | 20:4629784 |
| 87229 | 20:4630247 | 87230 | 20:4630301 | 87231 | 20:4630500 | 87232 | 20:4630563 | 87233 | 20:4630761 |
| 87234 | 20:4630879 | 87235 | 20:4630925 | 87236 | 20:4631015 | 87237 | 20:4631031 | 87238 | 20:4631157 |
| 87239 | 20:4631655 | 87240 | 20:4632251 | 87241 | 20:4632285 | 87242 | 20:4632908 | 87243 | 20:4633050 |
| 87244 | 20:4633087 | 87245 | 20:4633727 | 87246 | 20:4633738 | 87247 | 20:4633830 | 87248 | 20:4634166 |
| 87249 | 20:4634320 | 87250 | 20:4634619 | 87251 | 20:4634657 | 87252 | 20:4635386 | 87253 | 20:4635483 |
| 87254 | 20:4635990 | 87255 | 20:4637075 | 87256 | 20:4637125 | 87257 | 20:4638775 | 87258 | 20:4638910 |
| 87259 | 20:4639096 | 87260 | 20:4640118 | 87261 | 20:4640234 | 87262 | 20:4644059 | 87263 | 20:4644327 |
| 87264 | 20:4652157 | 87265 | 20:4654656 | 87267 | 20:4658089 | 87268 | 20:4658953 | 87269 | 20:4661324 |
| 87270 | 20:4665088 | 87271 | 20:4666775 | 87272 | 20:4667789 | 87275 | 20:4685771 | 87277 | 20:4690335 |
| 87278 | 20:4690908 | 87279 | 20:4691249 | 87280 | 20:4697066 | 87281 | 20:4697631 | 87282 | 20:4701728 |
| 87283 | 20:4701832 | 87284 | 20:4708012 | 87285 | 20:4709731 | 87286 | 20:4710087 | 87287 | 20:4712587 |
| 87288 | 20:4713160 | 87289 | 20:4715176 | 87290 | 20:4716043 | 87291 | 20:4726582 | 87292 | 20:4728514 |
| 87293 | 20:4730765 | 87294 | 20:4732330 | 87296 | 20:4736819 | 87297 | 20:4741050 | 87298 | 20:4750279 |
| 87299 | 20:4751162 | 87300 | 20:4752621 | 87301 | 20:4753160 | 87302 | 20:4757934 | 87303 | 20:4758722 |
| 87304 | 20:4761361 | 87305 | 20:4763955 | 87306 | 20:4770162 | 87309 | 20:4797785 | 87310 | 20:4805282 |
| 87311 | 20:4812157 | 87312 | 20:4819449 | 87314 | 20:4838678 | 87315 | 20:4841899 | 87316 | 20:4842019 |
| 87317 | 20:4846348 | 87318 | 20:4850816 | 87319 | 20:4851612 | 87320 | 20:4853215 | 87321 | 20:4854589 |
| 87322 | 20:4863651 | 87323 | 20:4873619 | 87324 | 20:4873645 | 87325 | 20:4873658 | 87326 | 20:4873667 |
| 87327 | 20:4880129 | 87328 | 20:4885163 | 87329 | 20:4922714 | 87330 | 20:4927232 | 87332 | 20:4928741 |
| 87333 | 20:4936772 | 87334 | 20:4937498 | 87335 | 20:4938011 | 87336 | 20:4938521 | 87337 | 20:4938780 |
| 87338 | 20:4938885 | 87339 | 20:4939294 | 87340 | 20:4940782 | 87341 | 20:4940805 | 87342 | 20:4944507 |
| 87343 | 20:4947601 | 87344 | 20:4949204 | 87345 | 20:4964757 | 87346 | 20:4965179 | 87347 | 20:4965760 |
| 87348 | 20:4969010 | 87349 | 20:4971347 | 87350 | 20:4971831 | 87351 | 20:4972329 | 87352 | 20:4974179 |
| 87354 | 20:4975337 | 87355 | 20:4978069 | 87356 | 20:4978213 | 87357 | 20:4978475 | 87359 | 20:4982228 |
| 87360 | 20:4984846 | 87361 | 20:4985523 | 87362 | 20:4986398 | 87363 | 20:4988142 | 87364 | 20:4990340 |
| 87365 | 20:4991225 | 87367 | 20:4992704 | 87368 | 20:4992712 | 87369 | 20:4993558 | 87370 | 20:4993669 |
| 87371 | 20:4993957 | 87373 | 20:4999299 | 87374 | 20:4999988 | 87376 | 20:5002715 | 87377 | 20:5003850 |
| 87378 | 20:5004638 | 87379 | 20:5008738 | 87380 | 20:5009598 | 87383 | 20:5040971 | 87385 | 20:5045805 |
| 87386 | 20:5049196 | 87387 | 20:5054905 | 87388 | 20:5055091 | 87389 | 20:5055857 | 87390 | 20:5058209 |
| 87391 | 20:5070761 | 87392 | 20:5074751 | 87393 | 20:5075106 | 87394 | 20:5076823 | 87395 | 20:5079687 |
| 87397 | 20:5081368 | 87399 | 20:5082509 | 87400 | 20:5107041 | 87402 | 20:5129728 | 87415 | 20:5282517 |
| 87429 | 20:5485968 | 87434 | 20:5633232 | 87435 | 20:5665364 | 87436 | 20:5695030 | 87438 | 20:5754805 |
| 87439 | 20:5796361 | 87449 | 20:6060819 | 87450 | 20:6062848 | 87451 | 20:6063007 | 87452 | 20:6063251 |
| 87453 | 20:6063553 | 87454 | 20:6063862 | 87455 | 20:6063984 | 87456 | 20:6064358 | 87457 | 20:6064442 |
| 87458 | 20:6066220 | 87459 | 20:6066792 | 87460 | 20:6066880 | 87461 | 20:6067197 | 87462 | 20:6069267 |
| 87463 | 20:6069326 | 87464 | 20:6069531 | 87465 | 20:6072588 | 87466 | 20:6073779 | 87467 | 20:6076915 |
| 87470 | 20:6090071 | 87477 | 20:6208301 | 87484 | 20:6325657 | 87488 | 20:6444472 | 87490 | 20:6526713 |
| 87509 | 20:6914755 | 87513 | 20:7017878 | 87516 | 20:7049088 | 87519 | 20:7079494 | 87521 | 20:7089351 |
| 87537 | 20:7437979 | 87539 | 20:7473329 | 87548 | 20:7706288 | 87550 | 20:7739561 | 87557 | 20:7958728 |
| 87559 | 20:7978859 | 87564 | 20:8099584 | 87566 | 20:8120294 | 87570 | 20:8190372 | 87573 | 20:8244125 |
| 87599 | 20:8976316 | 87608 | 20:9074647 | 87609 | 20:9107005 | 87662 | 20:10266518 | 87686 | 20:10946439 |
| 87698 | 20:11213830 | 87702 | 20:11274635 | 87711 | 20:11438125 | 87734 | 20:11948110 | 87736 | 20:12001911 |
| 87747 | 20:12228546 | 87750 | 20:12265622 | 87755 | 20:12409400 | 87758 | 20:12445726 | 87824 | 20:13931338 |
| 87826 | 20:13967565 | 87829 | 20:14050805 | 87830 | 20:14120849 | 87885 | 20:16058541 | 87923 | 20:17088438 |
| 87931 | 20:17251657 | 88103 | 20:21950333 | 88188 | 20:22481257 | 88190 | 20:22578241 | 88220 | 20:23180410 |
| 88233 | 20:23492042 | 88239 | 20:23575761 | 88241 | 20:23623587 | 88244 | 20:23695033 | 88334 | 20:25681663 |
| 88342 | 20:26011480 | 88352 | 20:26319951 | 88372 | 20:26721455 | 88379 | 20:26929602 | 88486 | 20:29337360 |
| 88501 | 20:29797692 | 88594 | 20:32271969 | 88629 | 20:33323766 | 88635 | 20:33477656 | 88636 | 20:33482995 |
| 88651 | 20:33788019 | 88843 | 20:38077292 | 88880 | 20:39076620 | 88893 | 20:39383171 | 88900 | 20:39458208 |
| 88901 | 20:39462501 | 88903 | 20:39469722 | 88906 | 20:39485628 | 88970 | 20:41005245 | 88987 | 20:41215061 |
| 89024 | 20:41990636 | 89201 | 20:46912182 | 89292 | 20:49691751 | 89327 | 20:50794831 | 89331 | 20:50908612 |
| 89334 | 20:50953384 | 89388 | 20:52906490 | 89424 | 20:53680094 | 89427 | 20:53779182 | 89490 | 20:55580535 |
| 89491 | 20:55615423 | 89501 | 20:55920256 | 89504 | 20:55944017 | 89603 | 20:58030736 | 89607 | 20:58078386 |
| 89748 | 20:61558977 | 89850 | 20:63825165 | 89852 | 20:63847390 | 89855 | 20:63882214 | 89947 | 20:65988032 |
| 89964 | 20:66468697 | 90010 | 20:67443129 | 90042 | 20:68014966 | 90105 | 20:69097447 | 90110 | 20:69174801 |
| 90114 | 20:69288227 | 90116 | 20:69317277 | 90161 | 20:70296851 | 90314 | 21:2109801 | 90321 | 21:2416354 |
| 90340 | 21:2937636 | 90422 | 21:5168513 | 90441 | 21:5776117 | 90447 | 21:5802937 | 90449 | 21:5865604 |
| 90452 | 21:5932063 | 90531 | 21:8044281 | 90549 | 21:8125084 | 90577 | 21:8739099 | 90673 | 21:10967078 |
| 90676 | 21:11012534 | 90870 | 21:15628004 | 90871 | 21:15629471 | 90872 | 21:15634222 | 90873 | 21:15635039 |
| 90874 | 21:15636617 | 90875 | 21:15640534 | 90876 | 21:15644415 | 90877 | 21:15648341 | 90878 | 21:15652428 |
| 90879 | 21:15656729 | 90964 | 21:17632245 | 90983 | 21:18074742 | 90987 | 21:18139041 | 91008 | 21:18709801 |
| 91009 | 21:18745959 | 91041 | 21:19240261 | 91119 | 21:20568083 | 91133 | 21:20667605 | 91163 | 21:21254778 |
| 91164 | 21:21278489 | 91244 | 21:22980503 | 91310 | 21:24894924 | 91377 | 21:26762300 | 91417 | 21:27993786 |
| 91435 | 21:28714736 | 91624 | 21:33373608 | 91661 | 21:33774652 | 91702 | 21:34416654 | 91768 | 21:35722374 |
| 91868 | 21:38504846 | 91877 | 21:38644972 | 91941 | 21:40541468 | 91985 | 21:41630642 | 92011 | 21:42193358 |
| 92020 | 21:42461759 | 92029 | 21:42678313 | 92115 | 21:44563811 | 92120 | 21:44677322 | 92171 | 21:46302745 |
| 92332 | 21:49387921 | 92335 | 21:49487176 | 92397 | 21:51289914 | 92407 | 21:51606765 | 92484 | 21:53761679 |
| 92488 | 21:53814553 | 92726 | 21:57261794 | 92848 | 21:59894217 | 92855 | 21:59981922 | 92967 | 21:62224560 |
| 93037 | 21:64128429 | 93155 | 21:66126088 | 93201 | 21:67026936 | 93296 | 21:69054783 | 93298 | 21:69111628 |
| 93301 | 21:69165607 | 93601 | 22:5786344 | 93653 | 22:6683071 | 93663 | 22:6874059 | 93668 | 22:6973667 |
| 93683 | 22:7347580 | 93785 | 22:10006376 | 93807 | 22:10285746 | 93819 | 22:10597656 | 93821 | 22:10648705 |
| 93829 | 22:10879669 | 93832 | 22:10961055 | 93894 | 22:12251220 | 94047 | 22:16130993 | 94110 | 22:17624753 |
| 94177 | 22:19212118 | 94255 | 22:21157473 | 94279 | 22:21651340 | 94283 | 22:21690013 | 94326 | 22:22930737 |
| 94380 | 22:24606457 | 94410 | 22:25532201 | 94504 | 22:28088504 | 94526 | 22:28449795 | 94614 | 22:30572708 |
| 94616 | 22:30617079 | 94621 | 22:30762175 | 94689 | 22:32709942 | 94691 | 22:32740432 | 94806 | 22:35660903 |
| 94908 | 22:38005262 | 94968 | 22:39268641 | 95000 | 22:39706378 | 95013 | 22:39906000 | 95037 | 22:40607655 |
| 95117 | 22:42731135 | 95121 | 22:42898400 | 95122 | 22:42936001 | 95284 | 22:46414503 | 95300 | 22:46810720 |
| 95307 | 22:46910055 | 95353 | 22:47726147 | 95583 | 22:53279742 | 95622 | 22:54013319 | 95666 | 22:54873498 |
| 95669 | 22:54947766 | 95676 | 22:55073957 | 95716 | 22:55626326 | 95781 | 22:56400223 | 95784 | 22:56416067 |
| 95785 | 22:56419658 | 95786 | 22:56420213 | 95809 | 22:56829490 | 95908 | 22:58476458 | 95913 | 22:58524949 |
| 95955 | 22:59413554 | 96087 | 23:2001057 | 96090 | 23:2028074 | 96118 | 23:2653886 | 96121 | 23:2672734 |
| 96124 | 23:2696666 | 96171 | 23:3661084 | 96177 | 23:3744075 | 96232 | 23:4789896 | 96422 | 23:8682465 |
| 96482 | 23:10137524 | 96546 | 23:11326784 | 96550 | 23:11418685 | 96552 | 23:11493234 | 96644 | 23:13147408 |
| 96647 | 23:13234890 | 96777 | 23:15576268 | 96872 | 23:17807855 | 96930 | 23:19250498 | 96931 | 23:19271779 |
| 96933 | 23:19307959 | 96941 | 23:19459539 | 96947 | 23:19483200 | 96959 | 23:19635143 | 96965 | 23:19711944 |
| 96996 | 23:20201294 | 97043 | 23:21249255 | 97051 | 23:21366692 | 97071 | 23:21956033 | 97096 | 23:22583782 |
| 97278 | 23:25346058 | 97357 | 23:27009758 | 97451 | 23:27999156 | 97501 | 23:28242781 | 97780 | 23:32969917 |
| 97828 | 23:34109165 | 97943 | 23:37547849 | 97981 | 23:38637301 | 98382 | 23:45418705 | 98389 | 23:45533685 |
| 98404 | 23:46021030 | 98438 | 23:46778964 | 98556 | 23:48912747 | 98631 | 23:50033982 | 98724 | 23:51773706 |
| 98783 | 24:520222 | 98832 | 24:1526726 | 98846 | 24:1793795 | 98892 | 24:2708333 | 98894 | 24:2766149 |
| 98913 | 24:3184889 | 98915 | 24:3208218 | 98935 | 24:3712541 | 98952 | 24:4048939 | 98964 | 24:4188152 |
| 98982 | 24:4612976 | 98994 | 24:5077959 | 99037 | 24:6230000 | 99125 | 24:8320822 | 99297 | 24:10714691 |
| 99353 | 24:12020111 | 99356 | 24:12116867 | 99361 | 24:12381665 | 99369 | 24:12616739 | 99402 | 24:13417942 |
| 99414 | 24:13675366 | 99451 | 24:14795404 | 99594 | 24:19151288 | 99665 | 24:20730048 | 99691 | 24:21214702 |
| 99863 | 24:25023821 | 99870 | 24:25259209 | 99898 | 24:25782545 | 99909 | 24:25992482 | 100009 | 24:28570245 |
| 100053 | 24:29754026 | 100059 | 24:29814575 | 100120 | 24:31415877 | 100199 | 24:32894188 | 100245 | 24:34122422 |
| 100257 | 24:34395443 | 100267 | 24:34719362 | 100285 | 24:35185903 | 100316 | 24:35736460 | 100323 | 24:35860154 |
| 100338 | 24:36195505 | 100358 | 24:37011897 | 100362 | 24:37166678 | 100364 | 24:37203930 | 100429 | 24:39199046 |
| 100782 | 24:46297155 | 100791 | 24:46409035 | 100919 | 24:49840655 | 100982 | 24:51488828 | 101005 | 24:51881818 |
| 101010 | 24:51956661 | 101014 | 24:52037619 | 101245 | 24:57052356 | 101347 | 24:59016719 | 101349 | 24:59106967 |
| 101394 | 24:60056629 | 101395 | 24:60093951 | 101469 | 24:61534805 | 101551 | 25:1163011 | 101605 | 25:1918071 |
| 101614 | 25:2105032 | 101618 | 25:2179579 | 101802 | 25:3997589 | 101849 | 25:4921820 | 101868 | 25:5339582 |
| 101958 | 25:6766372 | 101966 | 25:6952022 | 102005 | 25:7896632 | 102011 | 25:7949848 | 102018 | 25:8118349 |
| 102131 | 25:10832103 | 102149 | 25:11113745 | 102307 | 25:14593246 | 102318 | 25:14917112 | 102323 | 25:15012071 |
| 102329 | 25:15106926 | 102356 | 25:15663604 | 102365 | 25:15912161 | 102380 | 25:16148690 | 102399 | 25:16456322 |
| 102413 | 25:16841496 | 102459 | 25:17915319 | 102460 | 25:17952726 | 102462 | 25:18009869 | 102490 | 25:18825304 |
| 102523 | 25:19553139 | 102596 | 25:21400170 | 102612 | 25:21756847 | 102647 | 25:22463379 | 102656 | 25:22697194 |
| 102676 | 25:23209925 | 102679 | 25:23393931 | 102695 | 25:23751702 | 102713 | 25:24073747 | 102724 | 25:24300333 |
| 102732 | 25:24496560 | 102767 | 25:25119747 | 102774 | 25:25175370 | 102851 | 25:26611224 | 102875 | 25:27289405 |
| 102971 | 25:29170307 | 102973 | 25:29196073 | 102976 | 25:29227741 | 103031 | 25:30198863 | 103036 | 25:30292715 |
| 103045 | 25:30543167 | 103047 | 25:30590381 | 103165 | 25:33056358 | 103226 | 25:34314708 | 103243 | 25:34536825 |
| 103265 | 25:34878010 | 103273 | 25:35037480 | 103294 | 25:35416991 | 103369 | 25:36281889 | 103468 | 25:37785020 |
| 103522 | 25:38925016 | 103562 | 25:39799663 | 103598 | 25:40282487 | 103603 | 25:40362153 | 103698 | 25:42167568 |
| 103761 | 26:1999995 | 103766 | 26:2270743 | 103811 | 26:2874049 | 103964 | 26:7323964 | 104015 | 26:8514472 |
| 104061 | 26:9801561 | 104175 | 26:12542210 | 104214 | 26:13232302 | 104243 | 26:13935413 | 104244 | 26:13974845 |
| 104248 | 26:14058998 | 104265 | 26:14560437 | 104266 | 26:14562388 | 104420 | 26:18423545 | 104469 | 26:19395441 |
| 104485 | 26:19632302 | 104679 | 26:24855737 | 104714 | 26:25459976 | 104715 | 26:25463718 | 104716 | 26:25465826 |
| 104717 | 26:25467173 | 104781 | 26:26561752 | 104860 | 26:28504622 | 104925 | 26:30397842 | 104932 | 26:30643098 |
| 104935 | 26:30753391 | 104947 | 26:31085230 | 105378 | 26:41389219 | 105381 | 26:41451309 | 105394 | 26:41900876 |
| 105445 | 26:42982393 | 105499 | 26:44238502 | 105742 | 26:49454109 | 105754 | 26:49637614 | 105813 | 26:51001537 |
| 105824 | 26:51205579 | 105851 | 26:51911589 | 105854 | 26:51990348 | 105886 | 27:1292211 | 105932 | 27:2099758 |
| 105955 | 27:2545985 | 106036 | 27:4477596 | 106056 | 27:5041534 | 106064 | 27:5224821 | 106086 | 27:5489950 |
| 106115 | 27:6134492 | 106135 | 27:7463182 | 106203 | 27:8803096 | 106277 | 27:10171403 | 106312 | 27:11344637 |
| 106334 | 27:11976000 | 106374 | 27:12998392 | 106452 | 27:13945896 | 106510 | 27:15278766 | 106547 | 27:16013203 |
| 106553 | 27:16091265 | 106558 | 27:16161541 | 106608 | 27:16989882 | 106617 | 27:17229318 | 106655 | 27:18039702 |
| 106668 | 27:18444933 | 106672 | 27:18549440 | 106674 | 27:18561932 | 106695 | 27:18999723 | 106753 | 27:20126660 |
| 106800 | 27:21693284 | 106837 | 27:22606696 | 106839 | 27:22612038 | 106840 | 27:22625012 | 106841 | 27:22635635 |
| 106851 | 27:22872901 | 106892 | 27:23844184 | 106958 | 27:25097722 | 106963 | 27:25227473 | 107031 | 27:26703643 |
| 107032 | 27:26707702 | 107033 | 27:26709108 | 107034 | 27:26717112 | 107035 | 27:26752145 | 107037 | 27:26821155 |
| 107116 | 27:28602334 | 107142 | 27:29184261 | 107215 | 27:30821123 | 107219 | 27:30967912 | 107275 | 27:31920386 |
| 107418 | 27:35019984 | 107424 | 27:35045715 | 107434 | 27:35072059 | 107439 | 27:35098802 | 107452 | 27:35316815 |
| 107478 | 27:35710124 | 107512 | 27:36366267 | 107541 | 27:37168180 | 107629 | 27:38769358 | 107630 | 27:38784049 |
| 107632 | 27:38825952 | 107648 | 27:39079734 | 107650 | 27:39140091 | 107659 | 27:39414442 | 107684 | 27:40138920 |
| 107723 | 27:41177956 | 107855 | 27:44120630 | 107940 | 28:1026816 | 107943 | 28:1063432 | 107978 | 28:1669722 |
| 107979 | 28:1676667 | 107980 | 28:1688640 | 108035 | 28:3310204 | 108064 | 28:4807450 | 108072 | 28:5021515 |
| 108093 | 28:5430772 | 108094 | 28:5471888 | 108109 | 28:5837959 | 108120 | 28:5926093 | 108142 | 28:6534383 |
| 108248 | 28:9052050 | 108503 | 28:15552135 | 108505 | 28:15620196 | 108621 | 28:17961688 | 108634 | 28:18086730 |
| 108720 | 28:20471303 | 108761 | 28:21757902 | 108775 | 28:22091224 | 108882 | 28:24568402 | 108911 | 28:25196334 |
| 108950 | 28:26054541 | 108997 | 28:26764566 | 109005 | 28:26976598 | 109043 | 28:27784145 | 109225 | 28:31516271 |
| 109243 | 28:31960964 | 109334 | 28:33369552 | 109355 | 28:33823084 | 109363 | 28:34002468 | 109397 | 28:34663948 |
| 109420 | 28:34999208 | 109504 | 28:37068693 | 109511 | 28:37195142 | 109515 | 28:37311281 | 109519 | 28:37446774 |
| 109549 | 28:37921033 | 109557 | 28:38098897 | 109621 | 28:39341830 | 109741 | 28:40986070 | 109744 | 28:41014073 |
| 109881 | 28:43434313 | 109929 | 28:44311425 | 109935 | 28:44453574 | 109945 | 28:44780535 | 109958 | 28:44981684 |
| 109988 | 28:45527255 | 110035 | 29:1014606 | 110080 | 29:1997329 | 110082 | 29:2117573 | 110221 | 29:5006464 |
| 110233 | 29:5358686 | 110234 | 29:5358699 | 110248 | 29:5885429 | 110252 | 29:5979872 | 110429 | 29:10496324 |
| 110449 | 29:11034651 | 110590 | 29:14470262 | 110606 | 29:14866248 | 110721 | 29:17901471 | 110722 | 29:17951025 |
| 110723 | 29:17955354 | 110769 | 29:19855058 | 110770 | 29:19861520 | 110771 | 29:19862538 | 110985 | 29:25169363 |
| 110987 | 29:25193462 | 111011 | 29:25759092 | 111063 | 29:27485292 | 111143 | 29:28949555 | 111155 | 29:29347332 |
| 111211 | 29:30136039 | 111212 | 29:30157180 | 111221 | 29:30347114 | 111336 | 29:32940189 | 111340 | 29:33039863 |
| 111384 | 29:34046874 | 111390 | 29:34239637 | 111402 | 29:34523872 | 111422 | 29:34808241 | 111479 | 29:35951280 |
| 111481 | 29:35999607 | 111550 | 29:37304798 | 111551 | 29:37320062 | 111658 | 29:40811556 | 111659 | 29:40812741 |
| 111660 | 29:40813920 | 111661 | 29:40818497 | 111662 | 29:40820427 | 111663 | 29:40820963 | 111664 | 29:40821065 |
| 111666 | 29:40825872 | 111806 | 29:43407527 | 111808 | 29:43424442 | 111892 | 29:44721351 | 111901 | 29:45041058 |
| 111904 | 29:45172298 | 111928 | 29:45712362 | 111979 | 29:46833503 | 111989 | 29:46997866 | 111995 | 29:47125684 |
| 111996 | 29:47150455 | 112100 | 29:49234278 | 112124 | 29:49722188 | 112140 | 29:50217955 | 112141 | 29:50260533, |

and
a screening method of the sixth marker panel comprises the following steps: allowing genotyping results of the BovineHD chip of Illumina and the GGP Bovine 100K chip of Neogen in the Huaxi cattle population to have quality control at a minor allele frequency (MAF) of greater than 0.1, a genotype deletion rate of less than 10%, and a P value of Hardy-Weinberg equilibrium (HWE) testing of 1×10⁻⁶, and deleting invalid sites while retaining effective information marker sites in the Huaxi cattle population; and
the sites of the seventh marker panel are shown in the following table:

| NO. | Position | NO. | Position | NO. | Position | NO. | Position | NO. | Position |
|---|---|---|---|---|---|---|---|---|---|
| 1 | 1:198611 | 2 | 1:354105 | 3 | 1:413686 | 4 | 1:503436 | 5 | 1:539006 |
| 6 | 1:561519 | 8 | 1:752778 | 114 | 1:2548682 | 115 | 1:2645413 | 116 | 1:2738165 |
| 120 | 1:2859590 | 248 | 1:5217586 | 249 | 1:5308367 | 394 | 1:8387543 | 518 | 1:11192831 |
| 542 | 1:11918795 | 549 | 1:12187898 | 551 | 1:12295655 | 578 | 1:13034308 | 581 | 1:13135427 |
| 624 | 1:13936005 | 625 | 1:14033343 | 634 | 1:14325639 | 689 | 1:15749427 | 709 | 1:16317871 |
| 793 | 1:19108624 | 795 | 1:19246215 | 816 | 1:19761054 | 817 | 1:19840115 | 906 | 1:22225114 |
| 926 | 1:22716338 | 927 | 1:22813570 | 942 | 1:23156851 | 943 | 1:23255421 | 950 | 1:23587167 |
| 1081 | 1:27110041 | 1096 | 1:27690352 | 1099 | 1:27826641 | 1121 | 1:28708529 | 1184 | 1:30341410 |
| 1188 | 1:30639309 | 1228 | 1:31833724 | 1232 | 1:32038632 | 1239 | 1:32383066 | 1245 | 1:32604779 |
| 1302 | 1:34248126 | 1303 | 1:34341221 | 1336 | 1:35240562 | 1357 | 1:35661327 | 1373 | 1:36152445 |
| 1491 | 1:39146665 | 1500 | 1:39465349 | 1501 | 1:39555945 | 1528 | 1:40302747 | 1529 | 1:40392357 |
| 1555 | 1:41086735 | 1563 | 1:41385809 | 1565 | 1:41492237 | 1626 | 1:42693702 | 1627 | 1:42705366 |
| 1628 | 1:42797258 | 1769 | 1:45607475 | 1771 | 1:45687245 | 1772 | 1:45787243 | 1818 | 1:47287965 |
| 1838 | 1:47969845 | 1845 | 1:48250419 | 1876 | 1:49256468 | 1899 | 1:50064432 | 1921 | 1:50739583 |
| 1956 | 1:51942050 | 1961 | 1:52098640 | 2014 | 1:53514840 | 2090 | 1:54855775 | 2101 | 1:55175239 |
| 2117 | 1:55765709 | 2124 | 1:55975682 | 2125 | 1:56071064 | 2227 | 1:58399681 | 2228 | 1:58497604 |
| 2264 | 1:59389492 | 2269 | 1:59650598 | 2357 | 1:61707467 | 2364 | 1:61908265 | 2533 | 1:65639354 |
| 2534 | 1:65691897 | 2542 | 1:65975858 | 2553 | 1:66261981 | 2554 | 1:66349787 | 2575 | 1:66970472 |
| 2601 | 1:67667419 | 2612 | 1:68106244 | 2693 | 1:69418925 | 2702 | 1:69664149 | 2804 | 1:71695300 |
| 2825 | 1:72263210 | 2865 | 1:73046338 | 2912 | 1:74307455 | 2913 | 1:74402077 | 2942 | 1:75146396 |
| 2992 | 1:76510120 | 3001 | 1:76727873 | 3014 | 1:77141699 | 3091 | 1:78826134 | 3171 | 1:80384212 |
| 3239 | 1:81998130 | 3282 | 1:82911267 | 3295 | 1:83439908 | 3307 | 1:83899382 | 3321 | 1:84316332 |
| 3356 | 1:85642906 | 3372 | 1:86015500 | 3389 | 1:86437496 | 3390 | 1:86526242 | 3403 | 1:86930423 |
| 3415 | 1:87291852 | 3447 | 1:88278205 | 3448 | 1:88303482 | 3449 | 1:88477613 | 3450 | 1:88510142 |
| 3451 | 1:88605108 | 3480 | 1:89106677 | 3481 | 1:89184242 | 3605 | 1:92127457 | 3615 | 1:92303587 |
| 3627 | 1:92748568 | 3631 | 1:92911886 | 3662 | 1:93749721 | 3788 | 1:96724055 | 3898 | 1:99648631 |
| 3899 | 1:99745058 | 3907 | 1:99964213 | 3912 | 1:100151147 | 3951 | 1:101402516 | 3959 | 1:101604960 |
| 3975 | 1:102178820 | 3985 | 1:102499753 | 4011 | 1:103210827 | 4043 | 1:104445688 | 4047 | 1:104601851 |
| 4128 | 1:106679632 | 4215 | 1:108995880 | 4218 | 1:109138566 | 4223 | 1:109344061 | 4244 | 1:109812035 |
| 4303 | 1:110891497 | 4316 | 1:111431902 | 4391 | 1:113109748 | 4461 | 1:114671853 | 4507 | 1:116130150 |
| 4509 | 1:116269280 | 4510 | 1:116368715 | 4688 | 1:119719715 | 4739 | 1:120951015 | 4741 | 1:121018439 |
| 4742 | 1:121074170 | 4743 | 1:121144456 | 4745 | 1:121224156 | 4749 | 1:121384190 | 4759 | 1:121640449 |
| 4764 | 1:121788170 | 4774 | 1:122051101 | 4832 | 1:123574290 | 4833 | 1:123657947 | 4976 | 1:126688131 |
| 4977 | 1:126726689 | 4978 | 1:126823765 | 5040 | 1:128486623 | 5043 | 1:128606933 | 5093 | 1:129682985 |
| 5098 | 1:129814612 | 5099 | 1:129913741 | 5241 | 1:132429392 | 5247 | 1:132635025 | 5251 | 1:132828896 |
| 5260 | 1:133234769 | 5284 | 1:134043255 | 5414 | 1:136299174 | 5432 | 1:136970680 | 5444 | 1:137364879 |
| 5459 | 1:138082957 | 5460 | 1:138180043 | 5462 | 1:138259482 | 5464 | 1:138404453 | 5500 | 1:139245663 |
| 5502 | 1:139304863 | 5503 | 1:139403568 | 5511 | 1:139653452 | 5578 | 1:141255563 | 5701 | 1:143320922 |
| 5754 | 1:144299047 | 5884 | 1:146674083 | 5885 | 1:146773640 | 6509 | 1:157687863 | 6510 | 1:157775988 |
| 6512 | 1:157881491 | 6523 | 1:158316722 | 6524 | 1:158361834 | 6525 | 1:158460274 | 6527 | 2:97987 |
| 6528 | 2:156099 | 6529 | 2:251841 | 6609 | 2:1676588 | 6610 | 2:1762272 | 6627 | 2:2208427 |
| 6711 | 2:4459297 | 6819 | 2:6376890 | 6897 | 2:7947030 | 6898 | 2:7984204 | 6899 | 2:8067862 |
| 6921 | 2:8713754 | 6943 | 2:9417024 | 6945 | 2:9540631 | 6961 | 2:9879504 | 6962 | 2:9977673 |
| 6990 | 2:10739801 | 6994 | 2:11002563 | 7030 | 2:12006500 | 7035 | 2:12186267 | 7046 | 2:12679896 |
| 7117 | 2:14600625 | 7167 | 2:15807478 | 7249 | 2:17949516 | 7391 | 2:20586534 | 7405 | 2:20982246 |
| 7482 | 2:22953223 | 7490 | 2:23131851 | 7539 | 2:24040103 | 7590 | 2:25076321 | 7805 | 2:29677272 |
| 7836 | 2:30463341 | 7892 | 2:31678013 | 7912 | 2:32367398 | 7928 | 2:32964948 | 8001 | 2:34879480 |
| 8002 | 2:34940227 | 8003 | 2:35037207 | 8015 | 2:35478468 | 8021 | 2:35685945 | 8062 | 2:36835120 |
| 8185 | 2:40374618 | 8195 | 2:40590316 | 8364 | 2:44741204 | 8381 | 2:45256936 | 8524 | 2:48700283 |
| 8548 | 2:49196955 | 8550 | 2:49308768 | 8552 | 2:49502134 | 8577 | 2:50260674 | 8621 | 2:51619705 |
| 8622 | 2:51714357 | 8648 | 2:52249299 | 8665 | 2:52682136 | 8707 | 2:53776420 | 8717 | 2:54059294 |
| 8723 | 2:54299723 | 8817 | 2:56671164 | 8818 | 2:56766656 | 8859 | 2:57934056 | 8886 | 2:58700978 |
| 8907 | 2:59199942 | 8956 | 2:60394889 | 8972 | 2:60962014 | 9010 | 2:61641610 | 9011 | 2:61738618 |
| 9018 | 2:62012037 | 9023 | 2:62250012 | 9101 | 2:64316198 | 9116 | 2:64570288 | 9246 | 2:68272329 |
| 9258 | 2:68690400 | 9261 | 2:68901070 | 9264 | 2:69034698 | 9269 | 2:69214365 | 9274 | 2:69379425 |
| 9323 | 2:70760400 | 9362 | 2:71684921 | 9363 | 2:71782525 | 9365 | 2:71848257 | 9367 | 2:71970045 |
| 9406 | 2:73054461 | 9417 | 2:73255223 | 9483 | 2:75058828 | 9582 | 2:77785420 | 9651 | 2:79212662 |
| 9652 | 2:79232935 | 9655 | 2:79435179 | 9688 | 2:80269896 | 9691 | 2:80350552 | 9692 | 2:80443906 |
| 9718 | 2:81298125 | 9721 | 2:81552356 | 9731 | 2:82160162 | 9738 | 2:82525466 | 9741 | 2:82703708 |
| 9757 | 2:83398924 | 9759 | 2:83488349 | 9790 | 2:84070506 | 9793 | 2:84186911 | 9820 | 2:84735988 |
| 9822 | 2:84748768 | 9823 | 2:84845633 | 9848 | 2:85539387 | 9873 | 2:86113417 | 9878 | 2:86175227 |
| 9879 | 2:86265979 | 9908 | 2:86950234 | 9953 | 2:88328898 | 9980 | 2:88998896 | 10067 | 2:90047120 |
| 10069 | 2:90182742 | 10070 | 2:90271127 | 10071 | 2:90346277 | 10107 | 2:91439705 | 10170 | 2:92960490 |
| 10178 | 2:93253701 | 10243 | 2:94504077 | 10356 | 2:96858239 | 10362 | 2:97119985 | 10364 | 2:97284470 |
| 10374 | 2:97660903 | 10388 | 2:97949623 | 10453 | 2:99661757 | 10479 | 2:100148811 | 10510 | 2:101083832 |
| 10528 | 2:101669777 | 10534 | 2:101822460 | 10562 | 2:102640055 | 10642 | 2:104771582 | 10671 | 2:105313365 |
| 10916 | 2:110949976 | 10930 | 2:111452102 | 11063 | 2:114575713 | 11083 | 2:115163293 | 11148 | 2:116629100 |
| 11191 | 2:117806168 | 11194 | 2:117939332 | 11231 | 2:118820218 | 11232 | 2:118916665 | 11274 | 2:119675734 |
| 11302 | 2:120419474 | 11330 | 2:121151600 | 11342 | 2:121489660 | 11349 | 2:121623817 | 11350 | 2:121707578 |
| 11363 | 2:122105560 | 11448 | 2:124381727 | 11449 | 2:124471534 | 11500 | 2:125909343 | 11658 | 2:128226965 |
| 11765 | 2:130549076 | 11809 | 2:131471648 | 12088 | 2:135958653 | 12089 | 2:136042952 | 12096 | 2:136122518 |
| 12097 | 2:136218516 | 12103 | 3:192288 | 12172 | 3:1379812 | 12218 | 3:2245775 | 12219 | 3:2335779 |
| 12428 | 3:6422650 | 12571 | 3:8746180 | 12573 | 3:8864644 | 12579 | 3:9185006 | 12699 | 3:10683176 |
| 12753 | 3:11779967 | 12757 | 3:11849178 | 12758 | 3:11934240 | 12759 | 3:11998748 | 12811 | 3:13516552 |
| 12962 | 3:16327244 | 13006 | 3:17313416 | 13007 | 3:17408532 | 13036 | 3:18384479 | 13087 | 3:19384406 |
| 13088 | 3:19480721 | 13103 | 3:19938418 | 13104 | 3:19975811 | 13105 | 3:20062027 | 13114 | 3:20219541 |
| 13115 | 3:20275444 | 13134 | 3:20949426 | 13135 | 3:21041525 | 13139 | 3:21260248 | 13153 | 3:21701072 |
| 13164 | 3:21935930 | 13203 | 3:22637339 | 13253 | 3:23859633 | 13388 | 3:26747082 | 13484 | 3:28324475 |
| 13485 | 3:28415348 | 13486 | 3:28426219 | 13487 | 3:28519784 | 13535 | 3:29477079 | 14013 | 3:38769939 |
| 14015 | 3:38908661 | 14018 | 3:39010474 | 14019 | 3:39102130 | 14042 | 3:39825859 | 14046 | 3:39962964 |
| 14061 | 3:40312602 | 14077 | 3:40804040 | 14079 | 3:40921947 | 14112 | 3:41900576 | 14171 | 3:43138350 |
| 14186 | 3:43521434 | 14223 | 3:44490132 | 14224 | 3:44561235 | 14250 | 3:45348826 | 14257 | 3:45588541 |
| 14268 | 3:45973111 | 14270 | 3:46106986 | 14307 | 3:47132786 | 14310 | 3:47350830 | 14401 | 3:49233756 |
| 14490 | 3:50263076 | 14511 | 3:50885592 | 14519 | 3:51298193 | 14525 | 3:51591043 | 14548 | 3:52087305 |
| 14576 | 3:52675804 | 14622 | 3:53900489 | 14624 | 3:54093636 | 14635 | 3:54224439 | 14636 | 3:54256413 |
| 14637 | 3:54344299 | 14639 | 3:54400470 | 14640 | 3:54475982 | 14641 | 3:54564882 | 14642 | 3:54618300 |
| 14643 | 3:54697466 | 14644 | 3:54793255 | 14645 | 3:54888122 | 14679 | 3:55617345 | 14680 | 3:55712074 |
| 14682 | 3:55757845 | 14683 | 3:55857361 | 14699 | 3:56411615 | 14703 | 3:56614407 | 14724 | 3:57120778 |
| 14725 | 3:57210410 | 14730 | 3:57316623 | 14763 | 3:58252296 | 14807 | 3:59522050 | 14820 | 3:59954303 |
| 14878 | 3:61139535 | 14937 | 3:62995597 | 14986 | 3:64406567 | 15080 | 3:66682938 | 15081 | 3:66782644 |
| 15094 | 3:67125573 | 15179 | 3:69231454 | 15232 | 3:70770801 | 15263 | 3:72024952 | 15274 | 3:72399723 |
| 15277 | 3:72561296 | 15278 | 3:72658767 | 15282 | 3:72860663 | 15323 | 3:74163128 | 15334 | 3:74460656 |
| 15335 | 3:74560466 | 15373 | 3:75613962 | 15388 | 3:75959257 | 15396 | 3:76253283 | 15407 | 3:76546947 |
| 15518 | 3:79853923 | 15527 | 3:80073268 | 15551 | 3:80845482 | 15603 | 3:82127843 | 15647 | 3:83276598 |
| 15687 | 3:84374682 | 15749 | 3:85174617 | 15864 | 3:87868064 | 16131 | 3:93389797 | 16146 | 3:93816462 |
| 16173 | 3:94470905 | 16174 | 3:94570511 | 16205 | 3:95542077 | 16234 | 3:96375723 | 16236 | 3:96516771 |
| 16319 | 3:98506422 | 16487 | 3:101953130 | 16551 | 3:103287865 | 17051 | 3:112888751 | 17107 | 3:113766186 |
| 17462 | 3:119142510 | 17463 | 3:119241254 | 17465 | 3:119312183 | 17466 | 3:119337850 | 17471 | 3:119559889 |
| 17490 | 3:120011571 | 17505 | 3:120402369 | 17508 | 3:120529061 | 17527 | 4:196536 | 17528 | 4:280153 |
| 17533 | 4:535625 | 17539 | 4:791948 | 17549 | 4:1260383 | 17552 | 4:1417558 | 17600 | 4:2614737 |
| 17745 | 4:5912982 | 17995 | 4:11174189 | 18042 | 4:12473770 | 18087 | 4:13462303 | 18127 | 4:14704975 |
| 18131 | 4:14787436 | 18168 | 4:15690889 | 18178 | 4:16124536 | 18250 | 4:17859359 | 18322 | 4:19353388 |
| 18383 | 4:20445754 | 18433 | 4:21454127 | 18434 | 4:21541657 | 18445 | 4:21872378 | 18462 | 4:22281058 |
| 18501 | 4:23239526 | 18518 | 4:23775334 | 18529 | 4:24057792 | 18561 | 4:25063606 | 18581 | 4:25549415 |
| 18709 | 4:28892826 | 18713 | 4:29073099 | 18729 | 4:29642872 | 18740 | 4:30014475 | 18741 | 4:30101996 |
| 18846 | 4:32916549 | 18904 | 4:34496286 | 18926 | 4:35328524 | 19007 | 4:37643101 | 19054 | 4:38955171 |
| 19106 | 4:40318271 | 19156 | 4:41249802 | 19160 | 4:41529195 | 19161 | 4:41619769 | 19165 | 4:41769265 |
| 19169 | 4:41869434 | 19170 | 4:41950512 | 19184 | 4:42417863 | 19237 | 4:43892488 | 19281 | 4:45005168 |
| 19322 | 4:46242543 | 19325 | 4:46409911 | 19326 | 4:46490707 | 19407 | 4:48377180 | 19408 | 4:48467232 |
| 19487 | 4:50087185 | 19488 | 4:50186156 | 19499 | 4:50633232 | 19510 | 4:50996653 | 19537 | 4:51864991 |
| 19612 | 4:53951273 | 19613 | 4:54035561 | 19616 | 4:54191462 | 19618 | 4:54332820 | 19619 | 4:54391051 |
| 19629 | 4:54743084 | 19640 | 4:55160033 | 19642 | 4:55344926 | 19687 | 4:56506421 | 19766 | 4:59069599 |
| 19793 | 4:59746110 | 19805 | 4:60065778 | 19828 | 4:60746730 | 19861 | 4:61706369 | 19892 | 4:62405503 |
| 19897 | 4:62630427 | 19898 | 4:62713783 | 19987 | 4:65201660 | 19989 | 4:65245742 | 19990 | 4:65343359 |
| 19991 | 4:65436101 | 19993 | 4:65541264 | 20071 | 4:67301224 | 20088 | 4:67785324 | 20101 | 4:68075914 |
| 20135 | 4:69146493 | 20160 | 4:69711048 | 20161 | 4:69810925 | 20204 | 4:70819803 | 20231 | 4:71683652 |
| 20252 | 4:72269219 | 20253 | 4:72368269 | 20301 | 4:73885846 | 20309 | 4:74101412 | 20450 | 4:77371853 |
| 20475 | 4:77790836 | 20529 | 4:79467619 | 20647 | 4:82182401 | 20730 | 4:84549829 | 20733 | 4:84649340 |
| 20835 | 4:87133726 | 20859 | 4:87827889 | 20910 | 4:89303585 | 20944 | 4:90250683 | 20961 | 4:90701139 |
| 20978 | 4:91069757 | 21084 | 4:93203600 | 21136 | 4:94310305 | 21160 | 4:95074007 | 21254 | 4:96620212 |
| 21255 | 4:96718455 | 21485 | 4:99029738 | 21489 | 4:99310528 | 21491 | 4:99423168 | 21610 | 4:101894551 |
| 21616 | 4:102178027 | 21696 | 4:104152919 | 21762 | 4:105592184 | 21763 | 4:105606470 | 21764 | 4:105706248 |
| 21771 | 4:105878830 | 21772 | 4:105960151 | 21945 | 4:109512073 | 22167 | 4:113058361 | 22168 | 4:113156968 |
| 22546 | 4:119885612 | 22565 | 5:195803 | 22566 | 5:239346 | 22567 | 5:334945 | 22569 | 5:484209 |
| 22570 | 5:577221 | 22620 | 5:1547817 | 22706 | 5:3643809 | 22817 | 5:6128913 | 22818 | 5:6209013 |
| 23050 | 5:11616643 | 23133 | 5:13446994 | 23146 | 5:13786322 | 23170 | 5:14539492 | 23173 | 5:14638005 |
| 23183 | 5:14893917 | 23248 | 5:16007217 | 23258 | 5:16431067 | 23300 | 5:17571373 | 23303 | 5:17775869 |
| 23312 | 5:18094567 | 23332 | 5:18790466 | 23643 | 5:26034515 | 23644 | 5:26061374 | 23645 | 5:26151322 |
| 23657 | 5:26429773 | 23772 | 5:28790811 | 23781 | 5:29074134 | 23866 | 5:30986242 | 23867 | 5:31079915 |
| 23893 | 5:31837129 | 23983 | 5:34087072 | 23986 | 5:34286241 | 23987 | 5:34305687 | 23988 | 5:34375513 |
| 23989 | 5:34474025 | 23993 | 5:34687154 | 24020 | 5:35185968 | 24025 | 5:35351867 | 24034 | 5:35659101 |
| 24055 | 5:36320021 | 24070 | 5:36698940 | 24073 | 5:36843791 | 24100 | 5:37690831 | 24156 | 5:38824022 |
| 24222 | 5:40535193 | 24224 | 5:40667896 | 24269 | 5:42106898 | 24276 | 5:42475432 | 24298 | 5:42980132 |
| 24308 | 5:43193547 | 24330 | 5:43788410 | 24355 | 5:44185303 | 24356 | 5:44283317 | 24411 | 5:45870645 |
| 24496 | 5:47715438 | 24508 | 5:48068765 | 24588 | 5:50328496 | 24634 | 5:51301107 | 24681 | 5:52618260 |
| 24684 | 5:52742025 | 24687 | 5:52972061 | 24709 | 5:53722470 | 24719 | 5:53987712 | 24800 | 5:55749160 |
| 24856 | 5:57057069 | 24875 | 5:57516539 | 24885 | 5:57746077 | 24886 | 5:57832714 | 24887 | 5:57930196 |
| 24906 | 5:58383434 | 24909 | 5:58493473 | 24913 | 5:58628294 | 24914 | 5:58693980 | 24917 | 5:58889899 |
| 24918 | 5:58986587 | 24932 | 5:59477581 | 24935 | 5:59606022 | 24937 | 5:59692009 | 24938 | 5:59786270 |
| 24966 | 5:60382341 | 24967 | 5:60450259 | 25017 | 5:61851862 | 25115 | 5:64573229 | 25212 | 5:67004367 |
| 25214 | 5:67099597 | 25215 | 5:67183935 | 25245 | 5:67610309 | 25287 | 5:68696282 | 25352 | 5:70049824 |
| 25368 | 5:70612128 | 25480 | 5:72974385 | 25499 | 5:73530687 | 25534 | 5:74251346 | 25535 | 5:74343381 |
| 25593 | 5:75456468 | 25594 | 5:75473431 | 25595 | 5:75561363 | 25668 | 5:77473876 | 25669 | 5:77528763 |
| 25676 | 5:77903820 | 25690 | 5:78146870 | 25691 | 5:78242136 | 25730 | 5:79116954 | 25737 | 5:79370071 |
| 25738 | 5:79464943 | 25739 | 5:79516884 | 25762 | 5:80354597 | 25763 | 5:80452900 | 25786 | 5:81078405 |
| 25790 | 5:81259940 | 25866 | 5:82675565 | 25872 | 5:82889086 | 26084 | 5:84839297 | 26119 | 5:85681634 |
| 26136 | 5:86133720 | 26140 | 5:86239067 | 26194 | 5:87412820 | 26322 | 5:89485893 | 26392 | 5:91260025 |
| 26475 | 5:92686818 | 26708 | 5:97550215 | 26787 | 5:99116271 | 26788 | 5:99137116 | 26789 | 5:99143867 |
| 26790 | 5:99243441 | 26791 | 5:99314005 | 26931 | 5:102029541 | 26933 | 5:102150740 | 26938 | 5:102366923 |
| 26949 | 5:102638331 | 26950 | 5:102641378 | 26951 | 5:102662950 | 26952 | 5:102812214 | 26953 | 5:102878588 |
| 26954 | 5:102970619 | 26955 | 5:103010571 | 27120 | 5:106009983 | 27279 | 5:108384166 | 27298 | 5:108674540 |
| 27332 | 5:109217271 | 27333 | 5:109314750 | 27544 | 5:113514180 | 27545 | 5:113554598 | 27575 | 5:114440055 |
| 27576 | 5:114536143 | 27578 | 5:114640058 | 27595 | 5:115151924 | 27738 | 5:116870418 | 27938 | 6:97487 |
| 27946 | 6:394469 | 27947 | 6:492748 | 27979 | 6:1193084 | 28037 | 6:2239422 | 28060 | 6:2768563 |
| 28130 | 6:3837662 | 28184 | 6:5212458 | 28195 | 6:5517626 | 28196 | 6:5615037 | 28314 | 6:8143447 |
| 28328 | 6:8615351 | 28329 | 6:8684262 | 28437 | 6:11262662 | 28551 | 6:13520510 | 28553 | 6:13697509 |
| 28571 | 6:14245516 | 28744 | 6:17861677 | 28918 | 6:21562233 | 29086 | 6:25221507 | 29099 | 6:25661883 |
| 29133 | 6:26462640 | 29181 | 6:27717652 | 29384 | 6:31231051 | 29419 | 6:32134214 | 29424 | 6:32438836 |
| 29434 | 6:32698791 | 29437 | 6:32783759 | 29438 | 6:32866982 | 32354 | 6:44532925 | 32523 | 6:45804379 |
| 32559 | 6:46543851 | 32571 | 6:46849949 | 32625 | 6:48139435 | 32639 | 6:48619247 | 32722 | 6:50593610 |
| 32763 | 6:51940574 | 32772 | 6:52186933 | 32883 | 6:54324568 | 32961 | 6:55908779 | 32979 | 6:56522044 |
| 33028 | 6:57631443 | 33029 | 6:57728837 | 33054 | 6:58492711 | 33067 | 6:58822636 | 33096 | 6:59668604 |
| 33161 | 6:61063375 | 33203 | 6:62168026 | 33214 | 6:62509742 | 33219 | 6:62716293 | 33266 | 6:64072884 |
| 33270 | 6:64250794 | 33339 | 6:66605824 | 33359 | 6:67153345 | 33441 | 6:69518831 | 33443 | 6:69655680 |
| 33453 | 6:69965893 | 33454 | 6:70058818 | 33456 | 6:70166659 | 33473 | 6:70643646 | 33511 | 6:71802143 |
| 33591 | 6:74410889 | 33596 | 6:74629845 | 33599 | 6:74741424 | 33617 | 6:75350029 | 33619 | 6:75446552 |
| 33621 | 6:75621675 | 33636 | 6:75990894 | 33639 | 6:76209600 | 33640 | 6:76275919 | 33643 | 6:76450277 |
| 33706 | 6:78238972 | 33708 | 6:78363832 | 33713 | 6:78527838 | 33724 | 6:79038243 | 33745 | 6:79831508 |
| 33795 | 6:81332996 | 33807 | 6:81870711 | 33808 | 6:81964290 | 33862 | 6:83422593 | 33883 | 6:83749295 |
| 33906 | 6:84274386 | 33923 | 6:84775788 | 33989 | 6:86113898 | 34365 | 6:93730394 | 34406 | 6:94508641 |
| 34407 | 6:94604426 | 34408 | 6:94671907 | 34418 | 6:95161943 | 34422 | 6:95280868 | 34621 | 6:99278886 |
| 34645 | 6:100084140 | 34895 | 6:104783662 | 34926 | 6:105376492 | 35015 | 6:107674685 | 35016 | 6:107769675 |
| 35048 | 6:108791274 | 35098 | 6:109783321 | 35256 | 6:113049419 | 35257 | 6:113144284 | 35385 | 6:115461946 |
| 35450 | 6:116698963 | 35451 | 6:116752334 | 35452 | 6:116848436 | 35454 | 6:117023687 | 35455 | 6:117121869 |
| 35460 | 6:117289738 | 35461 | 6:117373173 | 35468 | 6:117512770 | 35471 | 6:117661215 | 35472 | 6:117698401 |
| 35473 | 6:117792640 | 35474 | 7:80741 | 35475 | 7:164546 | 35720 | 7:4691508 | 35922 | 7:7350133 |
| 35945 | 7:8005827 | 35954 | 7:8250300 | 35957 | 7:8423976 | 35962 | 7:8644832 | 35963 | 7:8715092 |
| 35964 | 7:8798532 | 35965 | 7:8829774 | 35967 | 7:9006597 | 35968 | 7:9034045 | 35969 | 7:9111685 |
| 35970 | 7:9205519 | 35971 | 7:9294350 | 35972 | 7:9323285 | 35973 | 7:9420386 | 35974 | 7:9427375 |
| 35975 | 7:9522686 | 35976 | 7:9608447 | 35980 | 7:9782711 | 35981 | 7:9853483 | 35982 | 7:9893322 |
| 35983 | 7:9935560 | 35987 | 7:10196676 | 35988 | 7:10292183 | 35993 | 7:10432630 | 35994 | 7:10494698 |
| 35995 | 7:10588854 | 36000 | 7:10694692 | 36001 | 7:10709248 | 36002 | 7:10799397 | 36003 | 7:10898948 |
| 36004 | 7:10972220 | 36034 | 7:11621411 | 36038 | 7:11733951 | 36290 | 7:15957756 | 36351 | 7:17374656 |
| 36373 | 7:17800196 | 36382 | 7:18107639 | 36530 | 7:20567856 | 36589 | 7:21886187 | 36612 | 7:22364050 |
| 36643 | 7:23141409 | 36693 | 7:24266298 | 36997 | 7:31102265 | 37168 | 7:34931021 | 37197 | 7:35685794 |
| 37213 | 7:36102653 | 37218 | 7:36242365 | 37331 | 7:38727405 | 37345 | 7:39057017 | 37432 | 7:40812159 |
| 37449 | 7:41102749 | 37450 | 7:41174068 | 37456 | 7:41391176 | 37460 | 7:41530820 | 37465 | 7:41621787 |
| 37485 | 7:42209152 | 37489 | 7:42394156 | 37545 | 7:43685836 | 37553 | 7:43941722 | 37559 | 7:44261005 |
| 37564 | 7:44505025 | 37616 | 7:45789210 | 37618 | 7:45899026 | 37622 | 7:46066923 | 37702 | 7:47884765 |
| 37704 | 7:47992422 | 37706 | 7:48152895 | 37757 | 7:49264671 | 37763 | 7:49508927 | 37764 | 7:49551981 |
| 37765 | 7:49632647 | 37766 | 7:49732360 | 37767 | 7:49825415 | 37772 | 7:50083648 | 37773 | 7:50141438 |
| 37774 | 7:50238296 | 37775 | 7:50295962 | 37776 | 7:50392227 | 37778 | 7:50523822 | 37782 | 7:50734140 |
| 37785 | 7:50857210 | 37786 | 7:50956025 | 37795 | 7:51298758 | 37805 | 7:51627529 | 37808 | 7:51770556 |
| 37810 | 7:51904626 | 37877 | 7:52516644 | 37913 | 7:53384544 | 37926 | 7:53682528 | 37936 | 7:54073527 |
| 38031 | 7:56472724 | 38061 | 7:57477755 | 38086 | 7:58105186 | 38087 | 7:58204392 | 38167 | 7:59670910 |
| 38195 | 7:60290921 | 38291 | 7:62241248 | 38363 | 7:63608853 | 38381 | 7:64051258 | 38502 | 7:66283774 |
| 38508 | 7:66535109 | 38570 | 7:68229559 | 38620 | 7:69315564 | 38662 | 7:70240253 | 38678 | 7:70856473 |
| 38773 | 7:73340932 | 38774 | 7:73425725 | 38792 | 7:73921199 | 38812 | 7:74461479 | 39002 | 7:78855864 |
| 39182 | 7:82082096 | 39313 | 7:85461499 | 39332 | 7:86158728 | 39339 | 7:86335874 | 39344 | 7:86445479 |
| 39366 | 7:86982142 | 39476 | 7:89945029 | 39477 | 7:90039805 | 39669 | 7:93367867 | 39716 | 7:94784594 |
| 40029 | 7:98642157 | 40031 | 7:98712459 | 40032 | 7:98793113 | 40136 | 7:101498466 | 40233 | 7:103750242 |
| 40234 | 7:103835986 | 40277 | 7:105099277 | 40483 | 7:110118836 | 40484 | 7:110198554 | 40485 | 7:110298545 |
| 40486 | 7:110364995 | 40487 | 7:110462011 | 40488 | 7:110524775 | 40489 | 7:110616075 | 40491 | 8:119077 |
| 40492 | 8:204055 | 40502 | 8:510254 | 40549 | 8:1447144 | 40550 | 8:1539600 | 40593 | 8:2298980 |
| 40618 | 8:3109616 | 40678 | 8:4419528 | 40679 | 8:4488605 | 40713 | 8:5356367 | 40792 | 8:7353398 |
| 40816 | 8:7715961 | 40887 | 8:9573171 | 40913 | 8:10172186 | 41018 | 8:12543611 | 41019 | 8:12642666 |
| 41081 | 8:14746899 | 41091 | 8:15001201 | 41101 | 8:15260009 | 41198 | 8:17406666 | 41267 | 8:19472727 |
| 41298 | 8:20253347 | 41380 | 8:22549276 | 41383 | 8:22747845 | 41388 | 8:22913643 | 41395 | 8:23313119 |
| 41446 | 8:24571671 | 41627 | 8:27179633 | 41661 | 8:28342382 | 41677 | 8:28819278 | 41709 | 8:29645861 |
| 41763 | 8:30913973 | 41814 | 8:32226362 | 41825 | 8:32643213 | 41830 | 8:32879092 | 41831 | 8:32974702 |
| 41833 | 8:33127475 | 41844 | 8:33407993 | 41845 | 8:33470771 | 41900 | 8:34950717 | 41905 | 8:35118252 |
| 41930 | 8:35658609 | 42020 | 8:37747312 | 42042 | 8:38560565 | 42053 | 8:38837520 | 42054 | 8:38936996 |
| 42058 | 8:39131324 | 42203 | 8:42579916 | 42214 | 8:42818923 | 42264 | 8:44335672 | 42287 | 8:45011011 |
| 42294 | 8:45233429 | 42414 | 8:47856381 | 42435 | 8:48611381 | 42541 | 8:51528439 | 42598 | 8:52866048 |
| 42705 | 8:55793930 | 42720 | 8:56175135 | 42793 | 8:57947632 | 42804 | 8:58378680 | 42806 | 8:58511840 |
| 42900 | 8:60710884 | 42937 | 8:61860162 | 42990 | 8:63095889 | 42996 | 8:63293941 | 42997 | 8:63391647 |
| 43046 | 8:64688332 | 43070 | 8:65351408 | 43083 | 8:65593070 | 43086 | 8:65766665 | 43108 | 8:66276105 |
| 43137 | 8:66960690 | 43199 | 8:68392696 | 43246 | 8:69535824 | 43279 | 8:70389972 | 43369 | 8:72702268 |
| 43466 | 8:75072947 | 43494 | 8:76094638 | 43555 | 8:77086614 | 43558 | 8:77243628 | 43569 | 8:77734663 |
| 43571 | 8:77844702 | 43601 | 8:78788179 | 43610 | 8:79140878 | 43615 | 8:79417890 | 43732 | 8:82254632 |
| 43780 | 8:83455155 | 43784 | 8:83630961 | 43864 | 8:85507118 | 43904 | 8:86456661 | 43907 | 8:86622904 |
| 43962 | 8:87959678 | 43976 | 8:88341264 | 44029 | 8:89430089 | 44104 | 8:91188437 | 44105 | 8:91284358 |
| 44119 | 8:91608918 | 44148 | 8:92091641 | 44201 | 8:93754674 | 44213 | 8:94306363 | 44214 | 8:94402192 |
| 44215 | 8:94469316 | 44280 | 8:96346983 | 44433 | 8:99864196 | 44481 | 8:101198784 | 44501 | 8:101698974 |
| 44683 | 8:104921232 | 44740 | 8:106178029 | 44742 | 8:106321176 | 44817 | 8:108369912 | 44818 | 8:108466305 |
| 44826 | 8:108830921 | 44827 | 8:108930028 | 44840 | 8:109399618 | 44844 | 8:109663372 | 44876 | 8:110607864 |
| 44902 | 8:111508670 | 44903 | 8:111524854 | 44904 | 8:111672008 | 44905 | 8:111767142 | 44906 | 8:111818202 |
| 44907 | 8:111891683 | 44908 | 8:111919894 | 44909 | 8:112058818 | 44910 | 8:112150362 | 44911 | 8:112232760 |
| 44912 | 8:112294473 | 44913 | 8:112461632 | 44914 | 8:112537285 | 44915 | 8:112604404 | 44916 | 8:112658175 |
| 44917 | 8:112746559 | 44918 | 8:112818233 | 44919 | 8:112865068 | 44920 | 8:112932415 | 44921 | 8:112969679 |
| 44922 | 8:112981176 | 44923 | 8:113097953 | 44924 | 8:113163827 | 44925 | 8:113180320 | 44926 | 8:113252524 |
| 44930 | 9:190038 | 44931 | 9:289864 | 45031 | 9:2539725 | 45074 | 9:3743248 | 45089 | 9:4213848 |
| 45112 | 9:4988015 | 45135 | 9:5683174 | 45179 | 9:7188401 | 45252 | 9:8906729 | 45274 | 9:9555271 |
| 45378 | 9:10911653 | 45502 | 9:13481091 | 45518 | 9:13965555 | 45582 | 9:15291327 | 45748 | 9:19358233 |
| 45798 | 9:20933619 | 45807 | 9:21235916 | 45808 | 9:21329366 | 46023 | 9:26340073 | 46029 | 9:26560747 |
| 46194 | 9:29938464 | 46280 | 9:31821673 | 46371 | 9:34153811 | 46384 | 9:34492853 | 46503 | 9:37375637 |
| 46515 | 9:37681606 | 46604 | 9:39416078 | 46605 | 9:39496518 | 46691 | 9:41269453 | 46692 | 9:41353434 |
| 46707 | 9:41741270 | 46708 | 9:41834289 | 46710 | 9:41949048 | 46773 | 9:43557020 | 46780 | 9:43780420 |
| 46834 | 9:45079509 | 46835 | 9:45168173 | 46839 | 9:45353183 | 46856 | 9:45976363 | 46867 | 9:46356308 |
| 46879 | 9:46772812 | 46892 | 9:47186026 | 46935 | 9:48538260 | 47033 | 9:51244082 | 47100 | 9:53108085 |
| 47125 | 9:53577095 | 47177 | 9:55074875 | 47178 | 9:55120285 | 47179 | 9:55219943 | 47205 | 9:56052958 |
| 47226 | 9:56606356 | 47254 | 9:57407794 | 47256 | 9:57496462 | 47281 | 9:58031623 | 47311 | 9:59018652 |
| 47408 | 9:61120108 | 47411 | 9:61356232 | 47442 | 9:62281541 | 47443 | 9:62376661 | 47493 | 9:63781683 |
| 47496 | 9:63979099 | 47499 | 9:64121913 | 47535 | 9:64946776 | 47643 | 9:66726405 | 47644 | 9:66806876 |
| 47646 | 9:66932759 | 47655 | 9:67186941 | 47715 | 9:68688996 | 47850 | 9:71852129 | 47857 | 9:72065985 |
| 47858 | 9:72161733 | 47894 | 9:73058318 | 47968 | 9:74896771 | 48054 | 9:77165874 | 48179 | 9:79135168 |
| 48188 | 9:79446217 | 48426 | 9:84561403 | 48431 | 9:84647624 | 48432 | 9:84745238 | 48521 | 9:86545282 |
| 48523 | 9:86670733 | 48544 | 9:87047352 | 48545 | 9:87102465 | 48546 | 9:87196437 | 48547 | 9:87295910 |
| 48615 | 9:88318893 | 48799 | 9:91914972 | 48879 | 9:93747256 | 49154 | 9:98379141 | 49230 | 9:99867971 |
| 49483 | 9:104102942 | 49484 | 9:104107451 | 49486 | 9:104142058 | 49488 | 9:104277105 | 49495 | 9:104453514 |
| 49497 | 9:104550915 | 49498 | 9:104641226 | 49499 | 9:104641308 | 49500 | 9:104641495 | 49502 | 10:184460 |
| 49503 | 10:213217 | 49504 | 10:295234 | 49506 | 10:344867 | 49507 | 10:435671 | 49566 | 10:1764102 |
| 49836 | 10:6862089 | 49850 | 10:7338516 | 49919 | 10:8633030 | 49991 | 10:10372137 | 50045 | 10:11267448 |
| 50115 | 10:12952255 | 50434 | 10:18833922 | 50435 | 10:18841050 | 50436 | 10:18845495 | 50437 | 10:18848616 |
| 50438 | 10:18854031 | 50469 | 10:19429779 | 50470 | 10:19516186 | 50638 | 10:22676353 | 50639 | 10:22775635 |
| 50642 | 10:22836164 | 50643 | 10:22909428 | 50646 | 10:23059842 | 50647 | 10:23066056 | 50648 | 10:23228975 |
| 50649 | 10:23268766 | 50650 | 10:23368614 | 50652 | 10:23464004 | 50653 | 10:23533419 | 50654 | 10:23627755 |
| 50655 | 10:23671760 | 50656 | 10:23769788 | 50657 | 10:23771464 | 50658 | 10:24790738 | 50659 | 10:24799735 |
| 50660 | 10:24818929 | 50661 | 10:24913820 | 50662 | 10:24923736 | 50663 | 10:25022602 | 50664 | 10:25057985 |
| 50665 | 10:25156372 | 50666 | 10:25164147 | 50667 | 10:25241806 | 50668 | 10:25341513 | 50680 | 10:25758684 |
| 50778 | 10:27556986 | 50900 | 10:29890120 | 50926 | 10:30807403 | 51080 | 10:34810626 | 51145 | 10:36132543 |
| 51146 | 10:36213908 | 51160 | 10:36596113 | 51163 | 10:36787409 | 51240 | 10:38618727 | 51259 | 10:39073733 |
| 51260 | 10:39169136 | 51284 | 10:39715696 | 51292 | 10:39993149 | 51302 | 10:40306626 | 51390 | 10:42652463 |
| 51403 | 10:43115839 | 51417 | 10:43643966 | 51433 | 10:44096569 | 51474 | 10:45124642 | 51490 | 10:45511746 |
| 51745 | 10:50796271 | 51861 | 10:53660012 | 51942 | 10:56043883 | 51992 | 10:57210937 | 52035 | 10:58319989 |
| 52040 | 10:58481438 | 52054 | 10:58874663 | 52071 | 10:59336872 | 52092 | 10:60028136 | 52123 | 10:61231374 |
| 52126 | 10:61443371 | 52158 | 10:62042620 | 52193 | 10:62708109 | 52228 | 10:63495064 | 52236 | 10:63804854 |
| 52247 | 10:64189747 | 52294 | 10:65251624 | 52312 | 10:65561116 | 52344 | 10:66259714 | 52392 | 10:67327408 |
| 52523 | 10:70522773 | 52524 | 10:70601203 | 52526 | 10:70690873 | 52527 | 10:70781098 | 52548 | 10:71377715 |
| 52590 | 10:72631813 | 52591 | 10:72727860 | 52596 | 10:72983865 | 52597 | 10:73076966 | 52643 | 10:74135935 |
| 52664 | 10:74941316 | 52700 | 10:75976569 | 52736 | 10:76707628 | 53015 | 10:82572013 | 53034 | 10:83144860 |
| 53169 | 10:86010466 | 53423 | 10:91110721 | 53454 | 10:92010714 | 53519 | 10:93279984 | 53520 | 10:93370297 |
| 53640 | 10:96340328 | 53705 | 10:97729030 | 53779 | 10:99321961 | 53802 | 10:99841614 | 53981 | 10:10327740 |
| 54006 | 11:514378 | 54266 | 11:4322857 | 54313 | 11:5395664 | 54364 | 11:6596735 | 54435 | 11:8259962 |
| 54438 | 11:8462700 | 54446 | 11:8761089 | 54705 | 11:14474472 | 54822 | 11:17458053 | 54838 | 11:17908654 |
| 54839 | 11:17983413 | 54843 | 11:18162535 | 54847 | 11:18333639 | 54889 | 11:19456930 | 54900 | 11:19732194 |
| 54920 | 11:20390071 | 54993 | 11:22091427 | 55041 | 11:23355951 | 55079 | 11:24188498 | 55126 | 11:25171864 |
| 55148 | 11:25792655 | 55180 | 11:26465214 | 55181 | 11:26466574 | 55182 | 11:26531940 | 55361 | 11:30575172 |
| 55422 | 11:32139468 | 55488 | 11:33907492 | 55506 | 11:34748337 | 55580 | 11:36801995 | 55638 | 11:37982330 |
| 55648 | 11:38441743 | 55686 | 11:39673530 | 55712 | 11:40436923 | 55715 | 11:40647883 | 55794 | 11:42338919 |
| 55853 | 11:43645607 | 55866 | 11:44133002 | 55918 | 11:45401293 | 55920 | 11:45518720 | 55922 | 11:45703344 |
| 55923 | 11:45779776 | 55956 | 11:46344663 | 56060 | 11:48391618 | 56182 | 11:50484454 | 56211 | 11:51437117 |
| 56233 | 11:52229998 | 56236 | 11:52492485 | 56254 | 11:53121751 | 56260 | 11:53366816 | 56270 | 11:53745957 |
| 56342 | 11:55279305 | 56373 | 11:55996334 | 56379 | 11:56241704 | 56381 | 11:56406566 | 56386 | 11:56550578 |
| 56393 | 11:56780643 | 56413 | 11:57282162 | 56423 | 11:57597675 | 56424 | 11:57696611 | 56449 | 11:58487002 |
| 56502 | 11:59891961 | 56507 | 11:60131924 | 56508 | 11:60226569 | 56583 | 11:61202324 | 56584 | 11:61289988 |
| 56586 | 11:61410046 | 56587 | 11:61495664 | 56592 | 11:61644339 | 56593 | 11:61726332 | 56717 | 11:64661124 |
| 56765 | 11:65925707 | 56800 | 11:66728012 | 56888 | 11:68240104 | 56900 | 11:68629264 | 56946 | 11:69472443 |
| 57044 | 11:70953054 | 57115 | 11:72333655 | 57123 | 11:72679475 | 57165 | 11:73412263 | 57169 | 11:73601533 |
| 57220 | 11:74558447 | 57221 | 11:74602747 | 57223 | 11:74706334 | 57268 | 11:75776010 | 57283 | 11:76197120 |
| 57467 | 11:79506772 | 57675 | 11:84372612 | 57684 | 11:84807135 | 57765 | 11:86512168 | 57810 | 11:87538574 |
| 57811 | 11:87624731 | 57982 | 11:90885707 | 58110 | 11:93370799 | 58146 | 11:94432248 | 58203 | 11:95957372 |
| 58367 | 11:99599606 | 58410 | 11:10044124 | 58478 | 11:10165527 | 58566 | 11:10321963 | 58606 | 11:10400943 |
| 58677 | 11:10536644 | 58680 | 11:10545702 | 58708 | 11:10585665 | 58709 | 11:10595532 | 58714 | 11:10618317 |
| 58716 | 11:10629012 | 58718 | 11:10643100 | 58721 | 11:10656647 | 58728 | 11:10671924 | 58730 | 11:10687276 |
| 58732 | 12:91462 | 58765 | 12:1113931 | 58768 | 12:1245380 | 58884 | 12:3705269 | 58963 | 12:5912555 |
| 58975 | 12:6345821 | 58982 | 12:6709902 | 58998 | 12:7126322 | 58999 | 12:7224038 | 59220 | 12:12834462 |
| 59327 | 12:15203798 | 59530 | 12:19527022 | 59607 | 12:21552423 | 59610 | 12:21691586 | 59611 | 12:21787162 |
| 59641 | 12:22535408 | 59688 | 12:23635155 | 59689 | 12:23728488 | 59791 | 12:25996134 | 59792 | 12:26092741 |
| 59794 | 12:26216578 | 59804 | 12:26581859 | 59875 | 12:28358077 | 59905 | 12:29299176 | 59926 | 12:29941712 |
| 59950 | 12:30491646 | 60005 | 12:31739889 | 60020 | 12:32049680 | 60093 | 12:33950251 | 60098 | 12:34160176 |
| 60110 | 12:34560071 | 60129 | 12:35057478 | 60167 | 12:35870799 | 60171 | 12:36164862 | 60174 | 12:36313336 |
| 60188 | 12:36646069 | 60194 | 12:36915655 | 60217 | 12:37496437 | 60218 | 12:37593149 | 60219 | 12:37662796 |
| 60221 | 12:37752580 | 60223 | 12:37929010 | 60224 | 12:37976297 | 60225 | 12:38053259 | 60230 | 12:38238813 |
| 60242 | 12:38895814 | 60247 | 12:39058185 | 60250 | 12:39172935 | 60271 | 12:39830361 | 60272 | 12:39927124 |
| 60297 | 12:40565431 | 60302 | 12:40827682 | 60314 | 12:41265419 | 60318 | 12:41435539 | 60322 | 12:41606679 |
| 60337 | 12:42104689 | 60343 | 12:42399827 | 60348 | 12:42626305 | 60349 | 12:42724793 | 60353 | 12:42919011 |
| 60357 | 12:43020517 | 60359 | 12:43190819 | 60369 | 12:43612489 | 60370 | 12:43698277 | 60374 | 12:43993681 |
| 60398 | 12:44816702 | 60406 | 12:45079833 | 60408 | 12:45170993 | 60432 | 12:45962853 | 60433 | 12:46045130 |
| 60515 | 12:48438301 | 60537 | 12:48906417 | 60622 | 12:50638774 | 60623 | 12:50712724 | 60795 | 12:54440430 |
| 60879 | 12:56915140 | 60896 | 12:57520936 | 60897 | 12:57618510 | 60917 | 12:58198920 | 60927 | 12:58558724 |
| 60980 | 12:60328402 | 60984 | 12:60431728 | 60991 | 12:60833351 | 60998 | 12:61166007 | 61017 | 12:61818463 |
| 61069 | 12:63224791 | 61091 | 12:63866391 | 61114 | 12:64636179 | 61130 | 12:65016774 | 61202 | 12:66945480 |
| 61203 | 12:67032666 | 61345 | 12:69830668 | 61346 | 12:70008764 | 61347 | 12:70092121 | 61349 | 12:70199557 |
| 61350 | 12:70296346 | 61351 | 12:70314066 | 61352 | 12:70373860 | 61354 | 12:70528567 | 61355 | 12:70625414 |
| 61356 | 12:70723529 | 61359 | 12:70754288 | 61360 | 12:70832160 | 61361 | 12:70921442 | 61362 | 12:71020218 |
| 61363 | 12:71027334 | 61364 | 12:71106543 | 61365 | 12:71199759 | 61375 | 12:71408795 | 61376 | 12:71457224 |
| 61377 | 12:71551210 | 61384 | 12:71729441 | 61392 | 12:71965489 | 61393 | 12:72058978 | 61394 | 12:72147420 |
| 61395 | 12:72230980 | 61396 | 12:72265152 | 61397 | 12:72337871 | 61398 | 12:72374872 | 61399 | 12:72490055 |
| 61400 | 12:72498314 | 61401 | 12:72637442 | 61402 | 12:72712776 | 61403 | 12:72812633 | 61902 | 12:83578230 |
| 62097 | 12:87141742 | 62098 | 13:299895 | 62105 | 13:682778 | 62106 | 13:745802 | 62125 | 13:1154063 |
| 62224 | 13:2881031 | 62225 | 13:2975142 | 62280 | 13:4395240 | 62283 | 13:4531236 | 62431 | 13:8202540 |
| 62466 | 13:9078686 | 62545 | 13:10762211 | 62546 | 13:10778845 | 62548 | 13:10867741 | 62554 | 13:11145952 |
| 62555 | 13:11222783 | 62556 | 13:11320371 | 62557 | 13:11410445 | 62570 | 13:12019870 | 62634 | 13:13812138 |
| 62650 | 13:14311743 | 62692 | 13:15465789 | 62712 | 13:16020130 | 62799 | 13:17568011 | 62801 | 13:17588165 |
| 62835 | 13:18353118 | 63019 | 13:21321597 | 63087 | 13:23067175 | 63093 | 13:23318050 | 63105 | 13:23791918 |
| 63109 | 13:23900966 | 63124 | 13:24343896 | 63189 | 13:25699872 | 63323 | 13:29049594 | 63392 | 13:30294381 |
| 63393 | 13:30377425 | 63476 | 13:31944092 | 63477 | 13:32039794 | 63555 | 13:33385230 | 63576 | 13:34100138 |
| 63577 | 13:34193250 | 63578 | 13:34248535 | 63581 | 13:34427463 | 63706 | 13:37210289 | 63805 | 13:39259881 |
| 63838 | 13:39814181 | 64020 | 13:43484075 | 64302 | 13:48427057 | 64315 | 13:49071500 | 64316 | 13:49168510 |
| 64345 | 13:49747887 | 64346 | 13:49844262 | 64355 | 13:50115519 | 64357 | 13:50180207 | 64358 | 13:50268295 |
| 64372 | 13:50739564 | 64374 | 13:50832795 | 64381 | 13:51125057 | 64401 | 13:51523719 | 64402 | 13:51622569 |
| 64500 | 13:53510732 | 64501 | 13:53607473 | 64507 | 13:53822879 | 64519 | 13:54442428 | 64570 | 13:55504923 |
| 64652 | 13:57623027 | 64676 | 13:58277988 | 64741 | 13:59446084 | 64816 | 13:61419709 | 64828 | 13:61686658 |
| 64836 | 13:61962725 | 64928 | 13:63467047 | 64929 | 13:63559780 | 64949 | 13:64081941 | 65039 | 13:65929411 |
| 65137 | 13:68477424 | 65180 | 13:69587905 | 65181 | 13:69666536 | 65541 | 13:77055890 | 65542 | 13:77153724 |
| 65879 | 14:93414 | 65895 | 14:609870 | 65904 | 14:947222 | 66104 | 14:4750888 | 66610 | 14:13439017 |
| 66611 | 14:13473101 | 66612 | 14:13553001 | 66613 | 14:13632606 | 66614 | 14:13721229 | 66615 | 14:13734707 |
| 66616 | 14:13832328 | 66786 | 14:17490496 | 66822 | 14:18129262 | 66823 | 14:18227619 | 66827 | 14:18277849 |
| 66828 | 14:18376690 | 66874 | 14:19576484 | 67559 | 14:25819002 | 67636 | 14:26880974 | 67665 | 14:27799331 |
| 67800 | 14:30971186 | 67869 | 14:32582584 | 67898 | 14:33242218 | 67971 | 14:34837449 | 67972 | 14:34885142 |
| 67977 | 14:35171625 | 68004 | 14:35658219 | 68094 | 14:38119848 | 68123 | 14:38980550 | 68157 | 14:40059476 |
| 68173 | 14:40339877 | 68272 | 14:43115419 | 68466 | 14:47686908 | 68471 | 14:47920443 | 68533 | 14:49442868 |
| 68573 | 14:50805531 | 68645 | 14:52567142 | 68650 | 14:52653514 | 68657 | 14:52955563 | 68658 | 14:53054564 |
| 68676 | 14:53669390 | 68747 | 14:54918170 | 68781 | 14:55712044 | 68782 | 14:55807243 | 68783 | 14:55875273 |
| 68784 | 14:55953452 | 68824 | 14:57218378 | 68935 | 14:59606995 | 68952 | 14:60129379 | 68980 | 14:60666003 |
| 68981 | 14:60764297 | 69127 | 14:63797690 | 69169 | 14:64906621 | 69173 | 14:65094231 | 69187 | 14:65545962 |
| 69327 | 14:68518147 | 69328 | 14:68615752 | 69461 | 14:72182566 | 69496 | 14:72949159 | 69497 | 14:72993679 |
| 69514 | 14:73377613 | 69551 | 14:74402579 | 69585 | 14:75284663 | 69654 | 14:76712535 | 69927 | 15:278408 |
| 69928 | 15:364919 | 69935 | 15:581520 | 69938 | 15:790710 | 69940 | 15:921595 | 70011 | 15:2907387 |
| 70072 | 15:4118742 | 70298 | 15:5868982 | 70344 | 15:7132196 | 70468 | 15:9855083 | 70479 | 15:10238469 |
| 70485 | 15:10450890 | 70487 | 15:10557012 | 70500 | 15:11034094 | 70513 | 15:11544670 | 70515 | 15:11646233 |
| 70535 | 15:12337840 | 70536 | 15:12426564 | 70558 | 15:13317083 | 70564 | 15:13540062 | 70607 | 15:14553709 |
| 70611 | 15:14685598 | 70652 | 15:15637037 | 70768 | 15:17775660 | 70799 | 15:18549454 | 70821 | 15:19204706 |
| 70830 | 15:19463500 | 71142 | 15:25686598 | 71348 | 15:29961188 | 71383 | 15:30812283 | 71652 | 15:36545261 |
| 71720 | 15:37976104 | 71736 | 15:38441326 | 71771 | 15:39152334 | 71772 | 15:39251802 | 71782 | 15:39467016 |
| 72064 | 15:44893395 | 72102 | 15:45703046 | 72116 | 15:46016293 | 72117 | 15:46052010 | 72118 | 15:46128678 |
| 72120 | 15:46190128 | 72193 | 15:47315722 | 72195 | 15:47377732 | 72196 | 15:47473423 | 72199 | 15:47598007 |
| 72204 | 15:47732907 | 72264 | 15:48782827 | 72293 | 15:49903002 | 72297 | 15:50131403 | 72298 | 15:50228681 |
| 72321 | 15:50928742 | 72446 | 15:53225186 | 72482 | 15:53576274 | 72512 | 15:54128239 | 72554 | 15:55076548 |
| 72599 | 15:56354181 | 72600 | 15:56425647 | 72782 | 15:59421385 | 72830 | 15:60713798 | 72933 | 15:62959638 |
| 73204 | 15:68655064 | 73251 | 15:70097809 | 73252 | 15:70187975 | 73651 | 15:78826872 | 73652 | 15:78926080 |
| 73661 | 15:79238876 | 73664 | 15:79380785 | 73669 | 15:79544197 | 73670 | 15:79611716 | 73785 | 15:82130501 |
| 73795 | 15:82404865 | 73796 | 15:82425557 | 73797 | 15:82522759 | 73884 | 15:84032423 | 73887 | 15:84244168 |
| 73888 | 15:84260381 | 73889 | 15:84356903 | 73890 | 15:84447429 | 73891 | 15:84540077 | 73894 | 15:84686045 |
| 73895 | 15:84729195 | 73896 | 15:84804908 | 73897 | 15:84896615 | 73898 | 15:84939269 | 73901 | 16:338529 |
| 74061 | 16:3596934 | 74075 | 16:3906912 | 74153 | 16:5495502 | 74156 | 16:5663689 | 74159 | 16:5838621 |
| 74167 | 16:6306111 | 74197 | 16:6971134 | 74198 | 16:7015739 | 74199 | 16:7111115 | 74201 | 16:7234019 |
| 74221 | 16:7834741 | 74224 | 16:7996266 | 74228 | 16:8149785 | 74229 | 16:8194291 | 74256 | 16:9122788 |
| 74257 | 16:9217866 | 74265 | 16:9575503 | 74293 | 16:10558307 | 74409 | 16:13263254 | 74425 | 16:13790388 |
| 74496 | 16:15729876 | 74548 | 16:17488453 | 74550 | 16:17617156 | 74566 | 16:18152045 | 74575 | 16:18477012 |
| 74632 | 16:19928511 | 74709 | 16:21880848 | 74750 | 16:22924908 | 74816 | 16:24474002 | 74979 | 16:27452942 |
| 75007 | 16:28071735 | 75037 | 16:29095796 | 75043 | 16:29327042 | 75168 | 16:32723586 | 75199 | 16:33327718 |
| 75372 | 16:37660699 | 75428 | 16:38784562 | 75449 | 16:39690828 | 75490 | 16:40983811 | 75550 | 16:42163425 |
| 75555 | 16:42310324 | 75556 | 16:42386477 | 75560 | 16:42697916 | 75568 | 16:42993444 | 75580 | 16:43487631 |
| 75582 | 16:43668103 | 75584 | 16:43759140 | 75585 | 16:43845132 | 75586 | 16:43944562 | 75588 | 16:44032421 |
| 75608 | 16:44623635 | 75612 | 16:44826869 | 75613 | 16:44926372 | 75614 | 16:45026368 | 75616 | 16:45103219 |
| 75627 | 16:45512990 | 75632 | 16:45776402 | 75637 | 16:46002437 | 75779 | 16:49103242 | 75849 | 16:51054365 |
| 75850 | 16:51151596 | 75859 | 16:51446273 | 75867 | 16:51646308 | 75868 | 16:51740534 | 75944 | 16:53407745 |
| 75997 | 16:54730701 | 76093 | 16:56989855 | 76409 | 16:64087780 | 76503 | 16:65762070 | 76697 | 16:69928868 |
| 76757 | 16:71345017 | 76835 | 16:72851827 | 77085 | 16:77598464 | 77222 | 16:79857865 | 77229 | 16:80072357 |
| 77230 | 16:80138235 | 77232 | 16:80311616 | 77233 | 16:80396660 | 77235 | 16:80531024 | 77236 | 16:80630465 |
| 77239 | 16:80799166 | 77242 | 16:80953675 | 77252 | 17:259333 | 77263 | 17:564310 | 77274 | 17:973466 |
| 77278 | 17:1054010 | 77487 | 17:5568436 | 77620 | 17:8228129 | 77681 | 17:9733872 | 77735 | 17:10897059 |
| 77736 | 17:10938025 | 77737 | 17:11020572 | 77738 | 17:11094236 | 77755 | 17:11494094 | 77830 | 17:13277982 |
| 78070 | 17:18481630 | 78136 | 17:19808398 | 78137 | 17:19897663 | 78207 | 17:21967677 | 78230 | 17:22786786 |
| 78249 | 17:23511334 | 78255 | 17:23800711 | 78322 | 17:25936343 | 78401 | 17:27754738 | 78430 | 17:28426405 |
| 78441 | 17:28791960 | 78462 | 17:29447559 | 78487 | 17:30217269 | 78545 | 17:31620341 | 78567 | 17:32254712 |
| 78570 | 17:32296225 | 78667 | 17:34617927 | 78688 | 17:35304907 | 78690 | 17:35390256 | 78721 | 17:36229501 |
| 78734 | 17:36837955 | 78744 | 17:37134508 | 78795 | 17:38760200 | 78814 | 17:39443909 | 78936 | 17:41933427 |
| 78937 | 17:42031727 | 78986 | 17:42956052 | 78987 | 17:43050836 | 79027 | 17:44108368 | 79108 | 17:46082443 |
| 79178 | 17:47678534 | 79293 | 17:50425399 | 79393 | 17:52219542 | 79410 | 17:52792109 | 79481 | 17:54629610 |
| 79511 | 17:55298739 | 79535 | 17:55992314 | 79545 | 17:56246007 | 79980 | 17:64743637 | 80106 | 17:67144833 |
| 80130 | 17:67822583 | 80279 | 17:70683155 | 80280 | 17:70709345 | 80281 | 17:70709461 | 80291 | 17:71094967 |
| 80383 | 17:72831459 | 80395 | 18:95447 | 80396 | 18:189110 | 80398 | 18:367703 | 80400 | 18:514106 |
| 80908 | 18:9808096 | 80994 | 18:11474377 | 81022 | 18:12273578 | 81074 | 18:13692264 | 81098 | 18:14306814 |
| 81099 | 18:14400626 | 81103 | 18:14549787 | 81105 | 18:14605010 | 81106 | 18:14636355 | 81107 | 18:14731173 |
| 81108 | 18:14801380 | 81151 | 18:15872181 | 81153 | 18:15986314 | 81159 | 18:16182872 | 81377 | 18:20732386 |
| 81736 | 18:28718247 | 81811 | 18:30779424 | 81813 | 18:30921028 | 81837 | 18:31639943 | 81954 | 18:34144938 |
| 82045 | 18:36256440 | 82052 | 18:36555965 | 82152 | 18:38719188 | 82233 | 18:40460799 | 82285 | 18:41570886 |
| 82432 | 18:44652242 | 82443 | 18:44987987 | 82600 | 18:47190873 | 82671 | 18:48450267 | 82706 | 18:49271447 |
| 82707 | 18:49370683 | 82738 | 18:50083459 | 82759 | 18:50626419 | 82760 | 18:50725650 | 82837 | 18:52240322 |
| 82862 | 18:53044750 | 82869 | 18:53322848 | 82997 | 18:55515927 | 83109 | 18:56926638 | 83227 | 18:58451447 |
| 83237 | 18:58961238 | 83240 | 18:59153638 | 83249 | 18:59423487 | 83250 | 18:59502600 | 83251 | 18:59597971 |
| 83252 | 18:59640740 | 83253 | 18:59738484 | 83254 | 18:59752260 | 83255 | 18:59841292 | 83271 | 18:60187977 |
| 83273 | 18:60349514 | 83293 | 18:61084174 | 83294 | 18:61180054 | 83301 | 18:61392112 | 83302 | 18:61484398 |
| 83373 | 18:62547161 | 83374 | 18:62548072 | 83375 | 18:62548590 | 83376 | 18:62614389 | 83386 | 18:63006555 |
| 83387 | 18:63077361 | 83422 | 18:63662692 | 83588 | 19:57925 | 83591 | 19:305379 | 83596 | 19:417884 |
| 83597 | 19:509378 | 83599 | 19:577296 | 83600 | 19:668256 | 83629 | 19:1655405 | 83656 | 19:2558450 |
| 83665 | 19:2953866 | 83671 | 19:3120033 | 83677 | 19:3275802 | 83714 | 19:4397168 | 83980 | 19:9821822 |
| 84056 | 19:11385109 | 84253 | 19:15470245 | 84549 | 19:20561916 | 84554 | 19:20740986 | 84572 | 19:21295269 |
| 84638 | 19:22476147 | 84674 | 19:23274892 | 84679 | 19:23407987 | 84688 | 19:23703085 | 84976 | 19:27081319 |
| 84977 | 19:27125583 | 84978 | 19:27182483 | 84979 | 19:27275569 | 84986 | 19:27665926 | 85115 | 19:30350931 |
| 85176 | 19:31793582 | 85191 | 19:32153081 | 85511 | 19:37954850 | 85578 | 19:39940442 | 85649 | 19:41359770 |
| 85749 | 19:43284000 | 85860 | 19:45535788 | 85910 | 19:46199205 | 85919 | 19:46495194 | 85936 | 19:46990600 |
| 86031 | 19:48855388 | 86072 | 19:49706256 | 86111 | 19:50303597 | 86113 | 19:50431879 | 86114 | 19:50531654 |
| 86241 | 19:52131137 | 86250 | 19:52418467 | 86565 | 19:56677337 | 86621 | 19:57762258 | 86622 | 19:57849833 |
| 86961 | 20:98572 | 86962 | 20:194921 | 87107 | 20:2918386 | 87430 | 20:5583730 | 87593 | 20:8760244 |
| 87840 | 20:14544398 | 87842 | 20:14662366 | 87845 | 20:14842827 | 87846 | 20:14894317 | 87848 | 20:15043520 |
| 87850 | 20:15190994 | 87888 | 20:16233299 | 87915 | 20:16830331 | 87920 | 20:17017002 | 87956 | 20:18017776 |
| 87971 | 20:18426404 | 88037 | 20:20313521 | 88073 | 20:21317972 | 88231 | 20:23439914 | 88321 | 20:25028667 |
| 88322 | 20:25092607 | 88327 | 20:25369162 | 88338 | 20:25827366 | 88357 | 20:26444773 | 88371 | 20:26712661 |
| 88493 | 20:29517107 | 88527 | 20:30497850 | 88541 | 20:31003971 | 88598 | 20:32503045 | 88599 | 20:32522681 |
| 88600 | 20:32622352 | 88602 | 20:32778020 | 88695 | 20:34788751 | 88842 | 20:38032426 | 88884 | 20:39213757 |
| 89063 | 20:43010466 | 89065 | 20:43140540 | 89068 | 20:43254253 | 89073 | 20:43352423 | 89105 | 20:44220967 |
| 89127 | 20:44701792 | 89143 | 20:45276596 | 89180 | 20:46098985 | 89181 | 20:46185509 | 89205 | 20:47109652 |
| 89218 | 20:47365415 | 89224 | 20:47651501 | 89232 | 20:47954545 | 89237 | 20:48129247 | 89238 | 20:48218836 |
| 89257 | 20:48716255 | 89281 | 20:49326846 | 89283 | 20:49443154 | 89319 | 20:50486303 | 89360 | 20:51882151 |
| 89375 | 20:52355899 | 89376 | 20:52454269 | 89400 | 20:53180123 | 89438 | 20:54174787 | 89439 | 20:54272031 |
| 89441 | 20:54375180 | 89450 | 20:54601936 | 89464 | 20:55005757 | 89492 | 20:55642905 | 89496 | 20:55834687 |
| 89710 | 20:60274323 | 89746 | 20:61511139 | 89881 | 20:64307221 | 90177 | 20:70744162 | 90271 | 20:71587457 |
| 90278 | 20:71850045 | 90283 | 21:134737 | 90284 | 21:233193 | 90286 | 21:309646 | 90287 | 21:371966 |
| 90288 | 21:471326 | 90289 | 21:506222 | 90291 | 21:638478 | 90292 | 21:726326 | 90293 | 21:823686 |
| 90294 | 21:904065 | 90295 | 21:966923 | 90296 | 21:1061609 | 90297 | 21:1093204 | 90298 | 21:1137598 |
| 90300 | 21:1255004 | 90301 | 21:1331973 | 90302 | 21:1425976 | 90303 | 21:1485463 | 90304 | 21:1581245 |
| 90306 | 21:1694504 | 90308 | 21:1850823 | 90309 | 21:1924655 | 90323 | 21:2437601 | 90347 | 21:3138288 |
| 90349 | 21:3228959 | 90358 | 21:3544839 | 90359 | 21:3610217 | 90364 | 21:3820824 | 90406 | 21:4613245 |
| 90440 | 21:5697639 | 90462 | 21:6211650 | 90464 | 21:6330904 | 90478 | 21:6814441 | 90482 | 21:6933120 |
| 90484 | 21:7029661 | 90640 | 21:10179845 | 91036 | 21:19148704 | 91170 | 21:21513912 | 91265 | 21:23382552 |
| 91266 | 21:23474228 | 91273 | 21:23715679 | 91285 | 21:24111132 | 91308 | 21:24835888 | 91334 | 21:25749419 |
| 91346 | 21:26098112 | 91360 | 21:26431821 | 91386 | 21:27005517 | 91387 | 21:27103067 | 91433 | 21:28660586 |
| 91552 | 21:31691093 | 91568 | 21:32209086 | 91593 | 21:32883760 | 91594 | 21:32927345 | 91595 | 21:33019785 |
| 91724 | 21:34773624 | 91725 | 21:34862867 | 91834 | 21:37259953 | 91836 | 21:37337560 | 91837 | 21:37418367 |
| 91858 | 21:38022062 | 91859 | 21:38118482 | 91861 | 21:38269475 | 91894 | 21:39232808 | 91946 | 21:40644524 |
| 91947 | 21:40743815 | 91953 | 21:40912381 | 91994 | 21:41910649 | 92018 | 21:42440438 | 92023 | 21:42514187 |
| 92102 | 21:44357068 | 92140 | 21:44936824 | 92141 | 21:45020642 | 92143 | 21:45198066 | 92152 | 21:45602636 |
| 92165 | 21:46016604 | 92166 | 21:46114894 | 92169 | 21:46249176 | 92297 | 21:48618715 | 92405 | 21:51567065 |
| 92449 | 21:52692952 | 92450 | 21:52792942 | 92453 | 21:52960457 | 92620 | 21:56228179 | 92621 | 21:56313681 |
| 92623 | 21:56441767 | 92814 | 21:59309517 | 92815 | 21:59375264 | 93006 | 21:63376882 | 93229 | 21:67773042 |
| 93338 | 22:94579 | 93352 | 22:646933 | 93358 | 22:824901 | 93437 | 22:2467585 | 93677 | 22:7210800 |
| 93714 | 22:8230735 | 93734 | 22:8758516 | 93765 | 22:9634144 | 93849 | 22:11365346 | 93942 | 22:13343219 |
| 93966 | 22:14036315 | 93997 | 22:14787281 | 94092 | 22:17058957 | 94135 | 22:18197695 | 94162 | 22:18799270 |
| 94176 | 22:19116049 | 94364 | 22:24074772 | 94470 | 22:27069723 | 94481 | 22:27482704 | 94563 | 22:29220441 |
| 94595 | 22:30012007 | 94650 | 22:31491877 | 94657 | 22:31704814 | 95074 | 22:41624457 | 95174 | 22:43984117 |
| 95228 | 22:45235116 | 95236 | 22:45525829 | 95393 | 22:48902067 | 95902 | 22:58335431 | 95953 | 22:59355819 |
| 96035 | 23:278582 | 96037 | 23:430451 | 96042 | 23:626975 | 96071 | 23:1548151 | 96072 | 23:1635007 |
| 96178 | 23:3824609 | 96286 | 23:5931144 | 96463 | 23:9588241 | 96613 | 23:12692439 | 96648 | 23:13322760 |
| 96655 | 23:13471246 | 96656 | 23:13531584 | 97061 | 23:21612939 | 97062 | 23:21712441 | 97063 | 23:21811769 |
| 97086 | 23:22310386 | 97103 | 23:22816656 | 97117 | 23:23369505 | 97336 | 23:25965936 | 97337 | 23:26013871 |
| 97338 | 23:26203805 | 97339 | 23:26302825 | 97340 | 23:26400435 | 97341 | 23:26404617 | 97342 | 23:26417922 |
| 97343 | 23:26516930 | 97346 | 23:26688460 | 97347 | 23:26750001 | 97348 | 23:26836934 | 97638 | 23:30204175 |
| 97639 | 23:30205302 | 97640 | 23:30205793 | 97675 | 23:30951436 | 97705 | 23:31625189 | 97851 | 23:34829880 |
| 97852 | 23:34888702 | 97859 | 23:35057295 | 97860 | 23:35151436 | 97889 | 23:36152667 | 97963 | 23:38043115 |
| 98233 | 23:42608340 | 98516 | 23:48087560 | 98519 | 23:48274159 | 98668 | 23:50824134 | 98767 | 23:52402327 |
| 98771 | 24:201134 | 98932 | 24:3597084 | 98987 | 24:4929150 | 99002 | 24:5203768 | 99108 | 24:7965661 |
| 99109 | 24:8063828 | 99131 | 24:8583177 | 99304 | 24:10935542 | 99358 | 24:12240445 | 99425 | 24:13974458 |
| 99478 | 24:15604573 | 99486 | 24:15989036 | 99487 | 24:16079054 | 99561 | 24:18229831 | 99572 | 24:18541933 |
| 99573 | 24:18640412 | 99606 | 24:19585603 | 99618 | 24:19942905 | 99624 | 24:20173407 | 99688 | 24:21131115 |
| 99764 | 24:22214747 | 99765 | 24:22312329 | 99816 | 24:23615391 | 99822 | 24:23975968 | 99826 | 24:24145037 |
| 99942 | 24:26860326 | 10005 | 24:29671134 | 10010 | 24:30883521 | 10010 | 24:30966522 | 10021 | 24:33423144 |
| 100223 | 24:33544520 | 10033 | 24:36290089 | 10034 | 24:36532147 | 10039 | 24:38166045 | 10040 | 24:38422210 |
| 100411 | 24:38722958 | 10047 | 24:40089318 | 10047 | 24:40188306 | 10054 | 24:41297881 | 10062 | 24:42939236 |
| 100630 | 24:43395685 | 10064 | 24:43924798 | 10064 | 24:44019504 | 10079 | 24:46654829 | 10085 | 24:48033977 |
| 100863 | 24:48144296 | 10087 | 24:48567652 | 10088 | 24:48858324 | 10101 | 24:52201248 | 10105 | 24:52902393 |
| 101118 | 24:54423570 | 10118 | 24:55890705 | 10120 | 24:56201929 | 10149 | 24:61932447 | 10150 | 24:62180714 |
| 101504 | 24:62277921 | 10150 | 25:94971 | 10161 | 25:2097812 | 10174 | 25:3066063 | 10199 | 25:7620341 |
| 102020 | 25:8255600 | 10204 | 25:8952928 | 10205 | 25:9079254 | 10228 | 25:14296254 | 10246 | 25:18216204 |
| 102662 | 25:22863656 | 10269 | 25:23717883 | 10285 | 25:26775859 | 10304 | 25:30451801 | 10304 | 25:30672143 |
| 103152 | 25:32745193 | 10360 | 25:40515689 | 10370 | 26:71749 | 10370 | 26:134754 | 10370 | 26:202644 |
| 103710 | 26:292215 | 10371 | 26:332815 | 10371 | 26:418885 | 10371 | 26:467022 | 10371 | 26:552651 |
| 103715 | 26:648778 | 10372 | 26:841636 | 10372 | 26:933342 | 10374 | 26:1410312 | 10376 | 26:2231655 |
| 103788 | 26:2771137 | 10388 | 26:4804367 | 10390 | 26:5316048 | 10391 | 26:5782436 | 10402 | 26:8899248 |
| 104047 | 26:9422315 | 10405 | 26:9629233 | 10413 | 26:11253125 | 10413 | 26:11345317 | 10413 | 26:11428247 |
| 104158 | 26:11886123 | 10426 | 26:14376252 | 10428 | 26:15092211 | 10428 | 26:15180609 | 10432 | 26:16278407 |
| 104330 | 26:16409629 | 10433 | 26:16616504 | 10448 | 26:19837144 | 10449 | 26:20063209 | 10450 | 26:20330629 |
| 104526 | 26:21059250 | 10461 | 26:23492556 | 10462 | 26:23837564 | 10463 | 26:23968846 | 10481 | 26:27340392 |
| 104865 | 26:28704657 | 10492 | 26:30296052 | 10493 | 26:30555507 | 10496 | 26:31506273 | 10510 | 26:34494728 |
| 105234 | 26:37659896 | 10530 | 26:39301430 | 10532 | 26:39903212 | 10538 | 26:41736203 | 10542 | 26:42498087 |
| 105426 | 26:42598014 | 10545 | 26:43157974 | 10552 | 26:44983809 | 10558 | 26:45965280 | 10575 | 26:49691984 |
| 105779 | 26:50189494 | 10578 | 26:50197785 | 10578 | 26:50203962 | 10578 | 26:50261536 | 10578 | 26:50350367 |
| 105784 | 26:50350409 | 10578 | 26:50414892 | 10585 | 27:99932 | 10585 | 27:181641 | 10585 | 27:250836 |
| 105858 | 27:316731 | 10585 | 27:394123 | 10586 | 27:443903 | 10586 | 27:543122 | 10586 | 27:642666 |
| 105867 | 27:739223 | 10586 | 27:838134 | 10586 | 27:928860 | 10587 | 27:1021674 | 10587 | 27:1119724 |
| 105923 | 27:1865958 | 10599 | 27:3535767 | 10599 | 27:3755867 | 10611 | 27:6215443 | 10611 | 27:6337782 |
| 106121 | 27:6434403 | 10612 | 27:6434442 | 10612 | 27:6621177 | 10612 | 27:6714941 | 10612 | 27:6754631 |
| 106126 | 27:6761335 | 10612 | 27:6952355 | 10612 | 27:6962879 | 10612 | 27:6980835 | 10613 | 27:6994129 |
| 106250 | 27:9301634 | 10626 | 27:9835607 | 10627 | 27:10239566 | 10632 | 27:11805253 | 10633 | 27:12112133 |
| 106347 | 27:12322130 | 10634 | 27:12386638 | 10645 | 27:14207575 | 10665 | 27:18065646 | 10666 | 27:18151157 |
| 106710 | 27:19373108 | 10676 | 27:20532211 | 10677 | 27:20833561 | 10682 | 27:22102480 | 10682 | 27:22221678 |
| 106829 | 27:22350322 | 10684 | 27:22785446 | 10699 | 27:26185311 | 10711 | 27:28416757 | 10718 | 27:30124180 |
| 107366 | 27:33574896 | 10737 | 27:34012998 | 10747 | 27:35694726 | 10753 | 27:36991890 | 10762 | 27:38609999 |
| 107654 | 27:39285504 | 10789 | 27:44800138 | 10791 | 27:45569924 | 10792 | 28:39225 | 10792 | 28:138659 |
| 107922 | 28:231989 | 10792 | 28:285448 | 10792 | 28:328039 | 10792 | 28:386583 | 10792 | 28:563052 |
| 107930 | 28:662354 | 10793 | 28:794289 | 10793 | 28:893452 | 10801 | 28:2643580 | 10802 | 28:2904232 |
| 108027 | 28:2999388 | 10803 | 28:3161546 | 10803 | 28:3253315 | 10803 | 28:3405359 | 10804 | 28:3759573 |
| 108043 | 28:3787485 | 10804 | 28:3885321 | 10804 | 28:4073833 | 10804 | 28:4163374 | 10805 | 28:4253300 |
| 108051 | 28:4338728 | 10805 | 28:4513285 | 10806 | 28:5004251 | 10818 | 28:7591484 | 10827 | 28:9724722 |
| 108313 | 28:10660380 | 10832 | 28:11111391 | 10832 | 28:11222161 | 10843 | 28:13814734 | 10854 | 28:16313184 |
| 108613 | 28:17683321 | 10863 | 28:18306254 | 10874 | 28:21104115 | 10877 | 28:22035988 | 10879 | 28:22784794 |
| 108801 | 28:22871308 | 10880 | 28:22889009 | 10884 | 28:23719431 | 10884 | 28:23801384 | 10902 | 28:27215263 |
| 109182 | 28:30404928 | 10933 | 28:33475488 | 10938 | 28:34421752 | 10943 | 28:35588714 | 10949 | 28:36727428 |
| 109624 | 28:39439768 | 10962 | 28:39494932 | 10963 | 28:39750515 | 10993 | 28:44515897 | 11000 | 29:178851 |
| 110009 | 29:265250 | 11001 | 29:607202 | 11007 | 29:1956188 | 11008 | 29:2091012 | 11023 | 29:5374583 |
| 110238 | 29:5456512 | 11023 | 29:5547355 | 11024 | 29:5627071 | 11024 | 29:5698810 | 11028 | 29:7011954 |
| 110333 | 29:8334191 | 11033 | 29:8529992 | 11054 | 29:13277373 | 11055 | 29:13442800 | 11058 | 29:14334295 |
| 110592 | 29:14552146 | 11066 | 29:16276745 | 11073 | 29:18298255 | 11074 | 29:18821433 | 11074 | 29:19176738 |
| 110756 | 29:19577395 | 11081 | 29:20938903 | 11081 | 29:21062969 | 11084 | 29:21741949 | 11102 | 29:26286827 |
| 111040 | 29:26637610 | 11104 | 29:26812835 | 11105 | 29:27123234 | 11105 | 29:27286001 | 11106 | 29:27336935 |
| 111061 | 29:27426724 | 11138 | 29:34145956 | 11151 | 29:36681488 | 11156 | 29:37610625 | 11157 | 29:37814400 |
| 111573 | 29:37903323 | 11157 | 29:37975002 | 11157 | 29:38065526 | 11157 | 29:38161198 | 11157 | 29:38251275 |
| 111578 | 29:38348064 | 11157 | 29:38414602 | 11158 | 29:38450767 | 11158 | 29:38467768 | 11158 | 29:38556318 |
| 111583 | 29:38649308 | 11158 | 29:38752213 | 11158 | 29:38847684 | 11158 | 29:38917900 | 11158 | 29:38952660 |
| 111590 | 29:38993981 | 11159 | 29:39083787 | 11159 | 29:39205805 | 11172 | 29:41919049 | 11214 | 29:50420995 |
| 112179 | 29:50977672 | 11218 | 29:51071432 | | | | | | . |

12. A method for genome-wide association study (GWAS) of Huaxi cattle using a genome-wide breeding chip of Huaxi cattle, wherein the genome-wide breeding chip of Huaxi cattle comprises a molecular marker combination;
wherein the molecular marker combination comprises seven marker panels; a first marker panel comprising 7,221 SNPs; a second marker panel comprising 22,937 SNPs and 1 Indel; a third marker panel comprising 249 SNPs; a fourth marker panel comprising 3,907 SNPs and 2 Indels; a fifth marker panel comprising 6,190 SNPs; a sixth marker panel comprising a total of 74,098 markers verified in a Huaxi cattle population from Illumina BovineHD chip and Neogen GGP Bovine 100K chip; a seventh marker panel comprising 2,617 SNPs; and an ARS-UCD 1.2/bosTau9 version is used as a reference genome of cattle;
the sites of the first marker panel are shown in the following table:

| NO. | Position | NO. | Position | NO. | Position | NO. | Position | NO. | Position |
|---|---|---|---|---|---|---|---|---|---|
| 27 | 1:1073001 | 40 | 1:1271108 | 124 | 1:3014460 | 164 | 1:3598559 | 213 | 1:4312806 |
| 216 | 1:4341656 | 257 | 1:5479286 | 286 | 1:6042789 | 314 | 1:6638827 | 324 | 1:6809931 |
| 386 | 1:8222113 | 399 | 1:8645362 | 407 | 1:8747963 | 480 | 1:10381445 | 484 | 1:10442662 |
| 547 | 1:12065998 | 575 | 1:12850062 | 586 | 1:13291614 | 593 | 1:13478803 | 594 | 1:13478804 |
| 595 | 1:13478805 | 600 | 1:13618910 | 602 | 1:13649839 | 603 | 1:13650350 | 604 | 1:13650573 |
| 605 | 1:13650578 | 606 | 1:13657524 | 607 | 1:13657759 | 608 | 1:13657853 | 609 | 1:13659104 |
| 610 | 1:13659735 | 611 | 1:13660313 | 612 | 1:13662505 | 613 | 1:13662879 | 614 | 1:13663313 |
| 615 | 1:13663369 | 632 | 1:14229816 | 635 | 1:14367624 | 640 | 1:14444223 | 646 | 1:14671195 |
| 675 | 1:15289219 | 732 | 1:17104389 | 745 | 1:17433162 | 761 | 1:18058788 | 779 | 1:18612790 |
| 803 | 1:19491923 | 807 | 1:19546032 | 831 | 1:20249479 | 855 | 1:20731028 | 866 | 1:21118209 |
| 901 | 1:22054771 | 936 | 1:23011477 | 954 | 1:23715328 | 957 | 1:23772692 | 974 | 1:24145046 |
| 1014 | 1:25285394 | 1015 | 1:25305166 | 1018 | 1:25448644 | 1038 | 1:25974291 | 1075 | 1:26927446 |
| 1089 | 1:27357800 | 1132 | 1:29040826 | 1142 | 1:29268481 | 1173 | 1:30001509 | 1176 | 1:30051285 |
| 1177 | 1:30052889 | 1206 | 1:31087002 | 1207 | 1:31090461 | 1211 | 1:31197827 | 1243 | 1:32461661 |
| 1255 | 1:32885018 | 1263 | 1:33160247 | 1384 | 1:36327051 | 1385 | 1:36335560 | 1404 | 1:36719216 |
| 1410 | 1:36920529 | 1412 | 1:36984896 | 1458 | 1:38181926 | 1480 | 1:38770057 | 1483 | 1:38899433 |
| 1495 | 1:39263861 | 1539 | 1:40637359 | 1546 | 1:40754498 | 1571 | 1:41666745 | 1617 | 1:42491489 |
| 1667 | 1:43750606 | 1687 | 1:44125197 | 1691 | 1:44260960 | 1710 | 1:44679137 | 1713 | 1:44805494 |
| 1719 | 1:44925165 | 1720 | 1:44925173 | 1721 | 1:44925749 | 1722 | 1:44927653 | 1723 | 1:44928339 |
| 1724 | 1:44928350 | 1725 | 1:44928489 | 1726 | 1:44929514 | 1727 | 1:44929625 | 1728 | 1:44929973 |
| 1729 | 1:44931081 | 1730 | 1:44931463 | 1753 | 1:45241871 | 1820 | 1:47384886 | 1828 | 1:47617677 |
| 1841 | 1:48116097 | 1963 | 1:52181284 | 1995 | 1:53016532 | 2003 | 1:53237486 | 2005 | 1:53252387 |
| 2006 | 1:53260493 | 2009 | 1:53326573 | 2013 | 1:53415161 | 2016 | 1:53539734 | 2030 | 1:53652726 |
| 2044 | 1:53770698 | 2051 | 1:54027097 | 2052 | 1:54039879 | 2053 | 1:54067827 | 2054 | 1:54068465 |
| 2062 | 1:54170560 | 2063 | 1:54175315 | 2073 | 1:54409914 | 2077 | 1:54504340 | 2078 | 1:54507288 |
| 2094 | 1:54985100 | 2100 | 1:55132890 | 2113 | 1:55602206 | 2115 | 1:55658614 | 2130 | 1:56173438 |
| 2133 | 1:56288769 | 2135 | 1:56366295 | 2136 | 1:56367389 | 2137 | 1:56367471 | 2140 | 1:56429824 |
| 2141 | 1:56439077 | 2151 | 1:56684792 | 2155 | 1:56767210 | 2164 | 1:56950851 | 2239 | 1:58810751 |
| 2254 | 1:59120478 | 2289 | 1:60052451 | 2299 | 1:60321205 | 2305 | 1:60534555 | 2314 | 1:60643904 |
| 2322 | 1:60871605 | 2331 | 1:61098681 | 2344 | 1:61320487 | 2345 | 1:61335565 | 2350 | 1:61476653 |
| 2374 | 1:62211191 | 2410 | 1:62916376 | 2411 | 1:62918132 | 2415 | 1:63017753 | 2416 | 1:63029817 |
| 2417 | 1:63033101 | 2418 | 1:63035068 | 2419 | 1:63037531 | 2420 | 1:63038112 | 2421 | 1:63038183 |
| 2422 | 1:63050131 | 2424 | 1:63119198 | 2425 | 1:63125457 | 2426 | 1:63128635 | 2427 | 1:63130293 |
| 2436 | 1:63393320 | 2450 | 1:63809663 | 2531 | 1:65592899 | 2574 | 1:66873010 | 2577 | 1:67103986 |
| 2584 | 1:67236736 | 2585 | 1:67260968 | 2636 | 1:68655539 | 2642 | 1:68743594 | 2664 | 1:68997540 |
| 2707 | 1:69843446 | 2722 | 1:70196536 | 2764 | 1:70655375 | 2776 | 1:70889623 | 2777 | 1:70925120 |
| 2811 | 1:71906639 | 2821 | 1:72100009 | 2896 | 1:73841520 | 2929 | 1:74779617 | 2950 | 1:75412538 |
| 2963 | 1:75770166 | 2964 | 1:75770197 | 2965 | 1:75770199 | 2966 | 1:75783854 | 2971 | 1:75829393 |
| 2984 | 1:76114883 | 3005 | 1:76860758 | 3042 | 1:77680697 | 3045 | 1:77701368 | 3070 | 1:78334604 |
| 3071 | 1:78335278 | 3075 | 1:78397021 | 3113 | 1:79354013 | 3224 | 1:81621771 | 3225 | 1:81658300 |
| 3262 | 1:82505239 | 3290 | 1:83265388 | 3301 | 1:83612627 | 3349 | 1:85328309 | 3362 | 1:85790627 |
| 3458 | 1:88779015 | 3466 | 1:88853263 | 3483 | 1:89288724 | 3506 | 1:89791299 | 3511 | 1:89828935 |
| 3523 | 1:90054999 | 3528 | 1:90169751 | 3539 | 1:90383988 | 3572 | 1:91171394 | 3584 | 1:91442961 |
| 3603 | 1:91945075 | 3608 | 1:92172948 | 3613 | 1:92213222 | 3618 | 1:92387065 | 3635 | 1:93023669 |
| 3663 | 1:93808595 | 3690 | 1:94443181 | 3742 | 1:95761493 | 3774 | 1:96387293 | 3818 | 1:97495906 |
| 3831 | 1:97875563 | 3835 | 1:97919421 | 3848 | 1:98319004 | 4084 | 1:105587286 | 4086 | 1:105625485 |
| 4088 | 1:105653161 | 4093 | 1:105729523 | 4163 | 1:107822647 | 4184 | 1:108388088 | 4193 | 1:108564048 |
| 4221 | 1:109261833 | 4235 | 1:109562630 | 4257 | 1:110017436 | 4317 | 1:111454247 | 4321 | 1:111612080 |
| 4346 | 1:112243802 | 4358 | 1:112481363 | 4360 | 1:112532754 | 4385 | 1:112920549 | 4398 | 1:113309692 |
| 4428 | 1:113860732 | 4469 | 1:114867292 | 4471 | 1:114870124 | 4500 | 1:115756884 | 4501 | 1:115772405 |
| 4512 | 1:116396361 | 4572 | 1:117632176 | 4674 | 1:119359964 | 4675 | 1:119363741 | 4707 | 1:120254924 |
| 4715 | 1:120408319 | 4716 | 1:120409192 | 4727 | 1:120576333 | 4737 | 1:120851703 | 4754 | 1:121481221 |
| 4758 | 1:121544917 | 4856 | 1:124230559 | 4872 | 1:124533841 | 4873 | 1:124538558 | 4889 | 1:124936627 |
| 4930 | 1:125655889 | 4933 | 1:125689194 | 4936 | 1:125738236 | 4941 | 1:125837099 | 4945 | 1:125909016 |
| 4952 | 1:126088394 | 5002 | 1:127555264 | 5013 | 1:127861573 | 5035 | 1:128339939 | 5048 | 1:128645880 |
| 5056 | 1:128748369 | 5218 | 1:131754936 | 5233 | 1:132161523 | 5273 | 1:133625347 | 5304 | 1:134472909 |
| 5333 | 1:135062430 | 5365 | 1:135657617 | 5404 | 1:135977700 | 5417 | 1:136372646 | 5458 | 1:137985176 |
| 5493 | 1:139072937 | 5525 | 1:140006988 | 5547 | 1:140601043 | 5559 | 1:140911883 | 5568 | 1:141070658 |
| 5577 | 1:141182724 | 5635 | 1:142058648 | 5639 | 1:142168313 | 5683 | 1:142908923 | 5952 | 1:148034123 |
| 5953 | 1:148040606 | 5954 | 1:148040677 | 5955 | 1:148044952 | 5956 | 1:148046284 | 5957 | 1:148046462 |
| 5958 | 1:148047932 | 5959 | 1:148053077 | 5960 | 1:148055822 | 5961 | 1:148058260 | 5962 | 1:148073345 |
| 5963 | 1:148073444 | 5964 | 1:148075396 | 5974 | 1:148329401 | 5980 | 1:148487864 | 6038 | 1:149409627 |
| 6039 | 1:149411937 | 6040 | 1:149413628 | 6045 | 1:149560052 | 6060 | 1:149819819 | 6139 | 1:151376946 |
| 6140 | 1:151406584 | 6141 | 1:151407059 | 6142 | 1:151407604 | 6152 | 1:151612724 | 6169 | 1:151767739 |
| 6171 | 1:151799413 | 6172 | 1:151799855 | 6173 | 1:151799970 | 6174 | 1:151800242 | 6175 | 1:151801164 |
| 6176 | 1:151801587 | 6177 | 1:151801737 | 6178 | 1:151801752 | 6191 | 1:152091262 | 6201 | 1:152265191 |
| 6202 | 1:152266056 | 6203 | 1:152266869 | 6204 | 1:152267613 | 6205 | 1:152267787 | 6206 | 1:152268300 |
| 6207 | 1:152268458 | 6213 | 1:152339615 | 6214 | 1:152343252 | 6216 | 1:152348515 | 6217 | 1:152349140 |
| 6219 | 1:152350298 | 6220 | 1:152351423 | 6221 | 1:152353474 | 6222 | 1:152353711 | 6223 | 1:152353915 |
| 6224 | 1:152354049 | 6225 | 1:152354217 | 6226 | 1:152354593 | 6227 | 1:152354711 | 6228 | 1:152355277 |
| 6229 | 1:152355296 | 6230 | 1:152355677 | 6231 | 1:152363064 | 6236 | 1:152422417 | 6266 | 1:152974350 |
| 6287 | 1:153459144 | 6356 | 1:154782431 | 6390 | 1:155509473 | 6395 | 1:155617067 | 6397 | 1:155639845 |
| 6398 | 1:155669488 | 6406 | 1:155709356 | 6410 | 1:155739527 | 6415 | 1:155837092 | 6420 | 1:155936300 |
| 6426 | 1:156024716 | 6437 | 1:156119189 | 6463 | 1:156640964 | 6466 | 1:156695097 | 6470 | 1:156760308 |
| 6486 | 1:157055516 | 6504 | 1:157454125 | 6507 | 1:157551305 | 6553 | 2:894974 | 6604 | 2:1577280 |
| 6679 | 2:3866542 | 6686 | 2:3991289 | 6688 | 2:4033904 | 6690 | 2:4040966 | 6691 | 2:4053609 |
| 6707 | 2:4311756 | 6747 | 2:5066365 | 6793 | 2:5931885 | 6795 | 2:5954528 | 6847 | 2:6974699 |
| 6858 | 2:7193712 | 6872 | 2:7370181 | 6879 | 2:7526996 | 6881 | 2:7574742 | 6942 | 2:9325130 |
| 6974 | 2:10332348 | 7018 | 2:11668736 | 7023 | 2:11736825 | 7056 | 2:13064383 | 7074 | 2:13460557 |
| 7080 | 2:13648131 | 7107 | 2:14339353 | 7122 | 2:14701151 | 7166 | 2:15712399 | 7233 | 2:17570676 |
| 7234 | 2:17577794 | 7308 | 2:18634499 | 7318 | 2:18907347 | 7319 | 2:18942177 | 7320 | 2:18943474 |
| 7321 | 2:18951656 | 7322 | 2:18958579 | 7323 | 2:18964610 | 7324 | 2:18967934 | 7333 | 2:19110921 |
| 7382 | 2:20297370 | 7385 | 2:20378241 | 7392 | 2:20658754 | 7396 | 2:20683165 | 7424 | 2:21454390 |
| 7453 | 2:22063850 | 7454 | 2:22086957 | 7457 | 2:22138573 | 7515 | 2:23573584 | 7538 | 2:24013539 |
| 7549 | 2:24246589 | 7565 | 2:24496758 | 7566 | 2:24500311 | 7567 | 2:24524614 | 7570 | 2:24563421 |
| 7588 | 2:24952433 | 7603 | 2:25454747 | 7645 | 2:26254049 | 7656 | 2:26496144 | 7725 | 2:27887225 |
| 7734 | 2:28111908 | 7757 | 2:28481427 | 7809 | 2:29740566 | 7881 | 2:31547829 | 7957 | 2:33826799 |
| 8010 | 2:35264773 | 8019 | 2:35614289 | 8041 | 2:36281226 | 8076 | 2:37317121 | 8084 | 2:37618680 |
| 8086 | 2:37625413 | 8229 | 2:41341923 | 8233 | 2:41443343 | 8234 | 2:41451445 | 8235 | 2:41463995 |
| 8236 | 2:41476600 | 8239 | 2:41525245 | 8240 | 2:41526634 | 8241 | 2:41546403 | 8242 | 2:41555839 |
| 8243 | 2:41555881 | 8244 | 2:41556165 | 8248 | 2:41609088 | 8249 | 2:41648397 | 8263 | 2:42040167 |
| 8264 | 2:42120013 | 8283 | 2:42489642 | 8284 | 2:42493609 | 8285 | 2:42495164 | 8313 | 2:43227689 |
| 8369 | 2:44933199 | 8396 | 2:45615535 | 8398 | 2:45632178 | 8409 | 2:45890270 | 8416 | 2:46041603 |
| 8493 | 2:47767849 | 8507 | 2:48158383 | 8511 | 2:48296454 | 8537 | 2:48966694 | 8542 | 2:48995422 |
| 8546 | 2:49074113 | 8587 | 2:50450300 | 8631 | 2:51841153 | 8632 | 2:51841281 | 8633 | 2:51845196 |
| 8634 | 2:51846388 | 8635 | 2:51848506 | 8636 | 2:51849168 | 8637 | 2:51850817 | 8638 | 2:51851321 |
| 8708 | 2:53799255 | 8721 | 2:54148820 | 8743 | 2:54689808 | 8751 | 2:54852723 | 8752 | 2:54874680 |
| 8786 | 2:55757496 | 8788 | 2:55856268 | 8836 | 2:57348471 | 8850 | 2:57670111 | 8867 | 2:58206752 |
| 8868 | 2:58209897 | 8870 | 2:58220436 | 8871 | 2:58224932 | 8872 | 2:58235017 | 8895 | 2:58894479 |
| 8937 | 2:59886642 | 8939 | 2:59894079 | 8941 | 2:59895422 | 8953 | 2:60262781 | 8986 | 2:61265032 |
| 9027 | 2:62337521 | 9064 | 2:63236556 | 9082 | 2:63698530 | 9103 | 2:64357206 | 9104 | 2:64367151 |
| 9106 | 2:64369812 | 9108 | 2:64437590 | 9109 | 2:64438167 | 9110 | 2:64438313 | 9111 | 2:64438576 |
| 9112 | 2:64438580 | 9113 | 2:64438635 | 9114 | 2:64438908 | 9122 | 2:64709919 | 9123 | 2:64711805 |
| 9144 | 2:65152676 | 9170 | 2:65933901 | 9210 | 2:67245827 | 9238 | 2:68102588 | 9241 | 2:68162816 |
| 9257 | 2:68641959 | 9282 | 2:69654612 | 9286 | 2:69797972 | 9289 | 2:69913137 | 9309 | 2:70434838 |
| 9326 | 2:70835144 | 9351 | 2:71366915 | 9354 | 2:71385196 | 9380 | 2:72368600 | 9431 | 2:73656519 |
| 9432 | 2:73692943 | 9438 | 2:73821323 | 9450 | 2:74104600 | 9460 | 2:74465077 | 9499 | 2:75629903 |
| 9522 | 2:76151639 | 9528 | 2:76318715 | 9572 | 2:77446117 | 9613 | 2:78673595 | 9630 | 2:78917434 |
| 9682 | 2:80074128 | 9733 | 2:82257633 | 9802 | 2:84430538 | 9875 | 2:86138420 | 9884 | 2:86394912 |
| 10120 | 2:91927539 | 10135 | 2:92191854 | 10175 | 2:93130350 | 10210 | 2:93703916 | 10252 | 2:94676418 |
| 10283 | 2:95426880 | 10287 | 2:95450849 | 10300 | 2:95820889 | 10323 | 2:96340062 | 10366 | 2:97323100 |
| 10378 | 2:97726887 | 10382 | 2:97746848 | 10396 | 2:98170549 | 10402 | 2:98356021 | 10418 | 2:98570804 |
| 10421 | 2:98681146 | 10424 | 2:98710984 | 10458 | 2:99816412 | 10462 | 2:99892472 | 10475 | 2:100046086 |
| 10476 | 2:100060098 | 10482 | 2:100180128 | 10493 | 2:100545847 | 10503 | 2:100804647 | 10519 | 2:101319592 |
| 10560 | 2:102476291 | 10585 | 2:103244299 | 10601 | 2:103704057 | 10604 | 2:103752303 | 10606 | 2:103798942 |
| 10619 | 2:104096932 | 10723 | 2:106585047 | 10741 | 2:106795862 | 10757 | 2:107198319 | 10759 | 2:107259324 |
| 10775 | 2:107639270 | 10795 | 2:107803660 | 10816 | 2:108400383 | 10844 | 2:108979070 | 10883 | 2:109843763 |
| 10889 | 2:109962178 | 10898 | 2:110266270 | 10908 | 2:110533827 | 10913 | 2:110782529 | 10921 | 2:111164890 |
| 10941 | 2:111820434 | 10980 | 2:112795307 | 11039 | 2:113925261 | 11047 | 2:114161924 | 11082 | 2:115099463 |
| 11086 | 2:115273767 | 11117 | 2:115778796 | 11118 | 2:115812040 | 11122 | 2:115883386 | 11129 | 2:116006798 |
| 11136 | 2:116182220 | 11144 | 2:116499676 | 11175 | 2:117182401 | 11219 | 2:118492898 | 11223 | 2:118587365 |
| 11228 | 2:118743982 | 11256 | 2:119406723 | 11265 | 2:119542337 | 11296 | 2:120281653 | 11318 | 2:120899441 |
| 11346 | 2:121587712 | 11400 | 2:123193388 | 11465 | 2:125068403 | 11477 | 2:125260143 | 11482 | 2:125405078 |
| 11524 | 2:126369897 | 11535 | 2:126645449 | 11566 | 2:126956287 | 11600 | 2:127060935 | 11611 | 2:127195966 |
| 11616 | 2:127259539 | 11643 | 2:127688853 | 11647 | 2:127843626 | 11660 | 2:128258137 | 11661 | 2:128269460 |
| 11739 | 2:129924952 | 11741 | 2:130000075 | 11795 | 2:131203320 | 11796 | 2:131203333 | 11797 | 2:131206099 |
| 11802 | 2:131272766 | 11848 | 2:132287345 | 11863 | 2:132630938 | 11864 | 2:132635825 | 11865 | 2:132637425 |
| 11922 | 2:133672767 | 11948 | 2:133927517 | 11962 | 2:134187671 | 11976 | 2:134370377 | 12003 | 2:134731903 |
| 12072 | 2:135676512 | 12100 | 3:74989 | 12107 | 3:258342 | 12138 | 3:907384 | 12182 | 3:1660018 |
| 12186 | 3:1722820 | 12267 | 3:3478015 | 12291 | 3:4097674 | 12293 | 3:4100027 | 12301 | 3:4206473 |
| 12342 | 3:4711535 | 12358 | 3:4936089 | 12364 | 3:5121045 | 12365 | 3:5121673 | 12366 | 3:5126160 |
| 12367 | 3:5130659 | 12386 | 3:5499346 | 12429 | 3:6474877 | 12436 | 3:6635006 | 12448 | 3:6773779 |
| 12474 | 3:7163463 | 12482 | 3:7308711 | 12520 | 3:7916080 | 12530 | 3:7997693 | 12589 | 3:9507918 |
| 12593 | 3:9562991 | 12600 | 3:9657041 | 12615 | 3:9990546 | 12621 | 3:10103527 | 12678 | 3:10496754 |
| 12726 | 3:11145282 | 12768 | 3:12417249 | 12776 | 3:12650225 | 12779 | 3:12749123 | 12804 | 3:13251625 |
| 12806 | 3:13370163 | 12807 | 3:13373604 | 12808 | 3:13375404 | 12812 | 3:13573971 | 12833 | 3:13902991 |
| 12866 | 3:14463788 | 12898 | 3:15197212 | 12926 | 3:15692187 | 12941 | 3:15905069 | 12972 | 3:16573676 |
| 12993 | 3:16936423 | 13136 | 3:21101166 | 13141 | 3:21359229 | 13158 | 3:21799171 | 13166 | 3:21978447 |
| 13170 | 3:22029801 | 13230 | 3:23274662 | 13295 | 3:24786196 | 13318 | 3:25281477 | 13329 | 3:25568097 |
| 13394 | 3:26947828 | 13404 | 3:27140090 | 13421 | 3:27417464 | 13426 | 3:27531689 | 13432 | 3:27586695 |
| 13433 | 3:27587568 | 13434 | 3:27588165 | 13435 | 3:27590766 | 13436 | 3:27590979 | 13437 | 3:27591026 |
| 13438 | 3:27591357 | 13439 | 3:27591992 | 13440 | 3:27592060 | 13441 | 3:27592156 | 13442 | 3:27592425 |
| 13443 | 3:27592963 | 13447 | 3:27618230 | 13448 | 3:27623256 | 13449 | 3:27624318 | 13450 | 3:27624378 |
| 13451 | 3:27626096 | 13488 | 3:28582812 | 13489 | 3:28655869 | 13493 | 3:28699418 | 13494 | 3:28701817 |
| 13496 | 3:28737754 | 13497 | 3:28739308 | 13502 | 3:28795698 | 13503 | 3:28847511 | 13512 | 3:29091766 |
| 13517 | 3:29129658 | 13528 | 3:29280162 | 13530 | 3:29285000 | 13543 | 3:29574496 | 13776 | 3:33446479 |
| 13782 | 3:33558867 | 13874 | 3:35055289 | 13935 | 3:36314410 | 13938 | 3:36323470 | 13942 | 3:36436943 |
| 13943 | 3:36535092 | 13955 | 3:36838325 | 13956 | 3:36846776 | 13971 | 3:37286638 | 14025 | 3:39287939 |
| 14031 | 3:39484220 | 14091 | 3:41318467 | 14096 | 3:41450661 | 14106 | 3:41608150 | 14116 | 3:41941631 |
| 14121 | 3:42042149 | 14131 | 3:42370072 | 14173 | 3:43226312 | 14205 | 3:43968451 | 14208 | 3:44136086 |
| 14252 | 3:45371773 | 14253 | 3:45389341 | 14259 | 3:45661559 | 14287 | 3:46609755 | 14319 | 3:47528437 |
| 14325 | 3:47614770 | 14335 | 3:47862102 | 14340 | 3:47901200 | 14346 | 3:48004343 | 14371 | 3:48662558 |
| 14376 | 3:48794169 | 14383 | 3:48908474 | 14388 | 3:48945044 | 14391 | 3:48996518 | 14393 | 3:49005907 |
| 14397 | 3:49066263 | 14400 | 3:49170559 | 14411 | 3:49437426 | 14430 | 3:49640985 | 14431 | 3:49686014 |
| 14432 | 3:49688113 | 14433 | 3:49693943 | 14434 | 3:49694205 | 14435 | 3:49694911 | 14436 | 3:49695537 |
| 14437 | 3:49695765 | 14438 | 3:49695863 | 14439 | 3:49695894 | 14440 | 3:49696034 | 14441 | 3:49697009 |
| 14442 | 3:49697975 | 14443 | 3:49698099 | 14444 | 3:49698127 | 14445 | 3:49698364 | 14446 | 3:49698389 |
| 14447 | 3:49698398 | 14448 | 3:49698433 | 14449 | 3:49698581 | 14450 | 3:49698599 | 14451 | 3:49698610 |
| 14452 | 3:49698697 | 14453 | 3:49698897 | 14454 | 3:49698954 | 14455 | 3:49698961 | 14456 | 3:49699145 |
| 14457 | 3:49699438 | 14458 | 3:49699942 | 14459 | 3:49700029 | 14460 | 3:49700320 | 14461 | 3:49700692 |
| 14462 | 3:49700695 | 14463 | 3:49700919 | 14464 | 3:49701129 | 14465 | 3:49701149 | 14466 | 3:49701225 |
| 14467 | 3:49701410 | 14468 | 3:49701470 | 14469 | 3:49702278 | 14470 | 3:49702301 | 14476 | 3:49768343 |
| 14489 | 3:50198427 | 14509 | 3:50849745 | 14526 | 3:51610373 | 14542 | 3:51914443 | 14546 | 3:51963753 |
| 14547 | 3:52005921 | 14550 | 3:52127559 | 14559 | 3:52354237 | 14560 | 3:52412518 | 14562 | 3:52416849 |
| 14563 | 3:52423182 | 14564 | 3:52427169 | 14566 | 3:52429848 | 14567 | 3:52433914 | 14570 | 3:52487075 |
| 14577 | 3:52690753 | 14580 | 3:52745287 | 14583 | 3:52852927 | 14591 | 3:53113743 | 14592 | 3:53117054 |
| 14593 | 3:53126092 | 14594 | 3:53126314 | 14595 | 3:53126332 | 14597 | 3:53143352 | 14600 | 3:53244194 |
| 14608 | 3:53474847 | 14610 | 3:53481481 | 14611 | 3:53510393 | 14652 | 3:55080090 | 14670 | 3:55424410 |
| 14740 | 3:57665967 | 14760 | 3:58213744 | 14761 | 3:58216986 | 14771 | 3:58501632 | 14789 | 3:58991070 |
| 14855 | 3:60699483 | 14856 | 3:60699729 | 14857 | 3:60704807 | 14871 | 3:60976891 | 14874 | 3:61002539 |
| 14876 | 3:61050627 | 14911 | 3:62289881 | 14936 | 3:62942816 | 14979 | 3:64119969 | 14994 | 3:64762588 |
| 15003 | 3:65001585 | 15011 | 3:65190175 | 15045 | 3:66062653 | 15056 | 3:66307909 | 15064 | 3:66462222 |
| 15108 | 3:67467438 | 15119 | 3:67681424 | 15160 | 3:68627230 | 15190 | 3:69504184 | 15196 | 3:69782248 |
| 15197 | 3:69782256 | 15198 | 3:69782262 | 15199 | 3:69786350 | 15215 | 3:70251128 | 15260 | 3:71848961 |
| 15262 | 3:71934380 | 15307 | 3:73666374 | 15325 | 3:74232466 | 15331 | 3:74366602 | 15394 | 3:76166365 |
| 15408 | 3:76630621 | 15458 | 3:78060366 | 15471 | 3:78544662 | 15539 | 3:80535834 | 15585 | 3:81521406 |
| 15586 | 3:81525295 | 15639 | 3:82930218 | 15642 | 3:83051313 | 15674 | 3:84083709 | 15679 | 3:84152801 |
| 15685 | 3:84286878 | 15713 | 3:84688663 | 15738 | 3:84955518 | 15745 | 3:85050052 | 15746 | 3:85066223 |
| 15766 | 3:85749188 | 15769 | 3:85769939 | 15774 | 3:85846088 | 15776 | 3:85897724 | 15784 | 3:85979828 |
| 15785 | 3:86001057 | 15786 | 3:86027116 | 15846 | 3:87314340 | 15854 | 3:87568457 | 15855 | 3:87570687 |
| 15857 | 3:87576354 | 15858 | 3:87576887 | 15859 | 3:87596560 | 15865 | 3:87888225 | 15900 | 3:88530363 |
| 15903 | 3:88603888 | 15905 | 3:88620871 | 15926 | 3:88972091 | 15934 | 3:89181812 | 15938 | 3:89279883 |
| 15955 | 3:89599405 | 15989 | 3:90306732 | 15990 | 3:90333561 | 16033 | 3:91219484 | 16036 | 3:91278796 |
| 16051 | 3:91695764 | 16084 | 3:92543467 | 16101 | 3:92905285 | 16113 | 3:93070586 | 16148 | 3:93889901 |
| 16213 | 3:95744017 | 16218 | 3:95893190 | 16222 | 3:96032684 | 16228 | 3:96151926 | 16281 | 3:97566938 |
| 16299 | 3:97901912 | 16346 | 3:99237470 | 16360 | 3:99532212 | 16362 | 3:99579446 | 16424 | 3:100686242 |
| 16429 | 3:100725968 | 16463 | 3:101430182 | 16476 | 3:101718735 | 16488 | 3:102039758 | 16507 | 3:102478118 |
| 16508 | 3:102487038 | 16525 | 3:102814655 | 16545 | 3:103051617 | 16547 | 3:103073339 | 16685 | 3:105603357 |
| 16698 | 3:105784125 | 16760 | 3:106768484 | 16763 | 3:106821154 | 16796 | 3:107517305 | 16797 | 3:107520457 |
| 16805 | 3:107754888 | 16842 | 3:108517017 | 16865 | 3:108844131 | 16904 | 3:109724431 | 16905 | 3:109725745 |
| 16906 | 3:109730150 | 16907 | 3:109731953 | 16908 | 3:109737608 | 16909 | 3:109737977 | 16910 | 3:109738917 |
| 16980 | 3:111463594 | 16984 | 3:111515807 | 17006 | 3:111954834 | 17039 | 3:112612031 | 17040 | 3:112625613 |
| 17055 | 3:112990876 | 17088 | 3:113561927 | 17123 | 3:114126601 | 17131 | 3:114256669 | 17145 | 3:114503215 |
| 17147 | 3:114519254 | 17151 | 3:114571821 | 17153 | 3:114600016 | 17154 | 3:114600017 | 17161 | 3:114722650 |
| 17203 | 3:115249589 | 17205 | 3:115271507 | 17250 | 3:116030538 | 17298 | 3:116861627 | 17300 | 3:116869634 |
| 17301 | 3:116871170 | 17303 | 3:116878333 | 17305 | 3:116901187 | 17306 | 3:116901527 | 17307 | 3:116903799 |
| 17311 | 3:116932009 | 17340 | 3:117275171 | 17356 | 3:117642247 | 17357 | 3:117642545 | 17358 | 3:117643811 |
| 17359 | 3:117644307 | 17398 | 3:118174177 | 17427 | 3:118632548 | 17445 | 3:118859735 | 17446 | 3:118864123 |
| 17451 | 3:118890670 | 17500 | 3:120280786 | 17594 | 4:2379436 | 17634 | 4:3851682 | 17635 | 4:3859753 |
| 17638 | 4:3982830 | 17645 | 4:4155060 | 17665 | 4:4566566 | 17667 | 4:4584480 | 17668 | 4:4589135 |
| 17669 | 4:4591392 | 17670 | 4:4591448 | 17677 | 4:4684194 | 17681 | 4:4780212 | 17682 | 4:4780800 |
| 17683 | 4:4786841 | 17687 | 4:4824857 | 17690 | 4:4865949 | 17694 | 4:4969955 | 17719 | 4:5368040 |
| 17727 | 4:5444338 | 17738 | 4:5704758 | 17765 | 4:6383685 | 17791 | 4:7064916 | 17803 | 4:7319609 |
| 17805 | 4:7325326 | 17815 | 4:7454936 | 17830 | 4:7796044 | 17846 | 4:8193002 | 17869 | 4:8790081 |
| 17881 | 4:9092344 | 17895 | 4:9372959 | 17903 | 4:9479800 | 17917 | 4:9747477 | 17933 | 4:9983918 |
| 17934 | 4:9983950 | 17935 | 4:9984121 | 17936 | 4:9984185 | 17937 | 4:9984248 | 17938 | 4:9984598 |
| 17940 | 4:9986515 | 17971 | 4:10645340 | 17974 | 4:10692378 | 17979 | 4:10807167 | 17980 | 4:10817042 |
| 17982 | 4:10817118 | 18028 | 4:11882269 | 18081 | 4:13277909 | 18083 | 4:13314057 | 18084 | 4:13315343 |
| 18132 | 4:14870332 | 18185 | 4:16314407 | 18193 | 4:16510166 | 18212 | 4:17151245 | 18220 | 4:17227279 |
| 18227 | 4:17429209 | 18244 | 4:17664281 | 18251 | 4:17934274 | 18292 | 4:18879751 | 18295 | 4:18927680 |
| 18301 | 4:18998283 | 18302 | 4:18999825 | 18303 | 4:19004726 | 18304 | 4:19005718 | 18305 | 4:19006333 |
| 18306 | 4:19007854 | 18307 | 4:19007876 | 18308 | 4:19008526 | 18309 | 4:19008760 | 18310 | 4:19010468 |
| 18331 | 4:19684498 | 18332 | 4:19687001 | 18333 | 4:19687703 | 18334 | 4:19688039 | 18335 | 4:19690469 |
| 18336 | 4:19690711 | 18337 | 4:19692144 | 18338 | 4:19692593 | 18339 | 4:19692676 | 18340 | 4:19693098 |
| 18341 | 4:19693119 | 18342 | 4:19694330 | 18343 | 4:19694937 | 18344 | 4:19695576 | 18345 | 4:19695681 |
| 18346 | 4:19696720 | 18347 | 4:19696767 | 18348 | 4:19697089 | 18349 | 4:19697301 | 18350 | 4:19697430 |
| 18351 | 4:19697544 | 18352 | 4:19697663 | 18354 | 4:19748494 | 18359 | 4:19938692 | 18361 | 4:19989444 |
| 18375 | 4:20261489 | 18376 | 4:20265073 | 18392 | 4:20703476 | 18401 | 4:20822864 | 18421 | 4:21167294 |
| 18431 | 4:21342092 | 18432 | 4:21358199 | 18440 | 4:21699344 | 18457 | 4:22138635 | 18466 | 4:22471078 |
| 18468 | 4:22523960 | 18481 | 4:22728193 | 18490 | 4:22923982 | 18497 | 4:23121119 | 18500 | 4:23196768 |
| 18504 | 4:23389777 | 18530 | 4:24084837 | 18546 | 4:24614792 | 18563 | 4:25092475 | 18600 | 4:26062712 |
| 18625 | 4:26743642 | 18628 | 4:26796377 | 18632 | 4:26851816 | 18643 | 4:27158677 | 18656 | 4:27519461 |
| 18679 | 4:28114813 | 18697 | 4:28590705 | 18717 | 4:29227466 | 18733 | 4:29791373 | 18759 | 4:30557284 |
| 18768 | 4:30768290 | 18782 | 4:31109031 | 18806 | 4:31790626 | 18808 | 4:31802602 | 18827 | 4:32352959 |
| 18838 | 4:32687897 | 18870 | 4:33603343 | 18902 | 4:34379775 | 18952 | 4:36053918 | 18953 | 4:36057890 |
| 18966 | 4:36473548 | 18993 | 4:37223623 | 19001 | 4:37381316 | 19019 | 4:38054717 | 19024 | 4:38091098 |
| 19030 | 4:38294871 | 19041 | 4:38579078 | 19042 | 4:38591331 | 19060 | 4:39231026 | 19066 | 4:39400801 |
| 19084 | 4:39970721 | 19114 | 4:40595576 | 19133 | 4:40929985 | 19178 | 4:42197758 | 19207 | 4:43094045 |
| 19209 | 4:43138220 | 19236 | 4:43793806 | 19294 | 4:45527845 | 19336 | 4:46745755 | 19366 | 4:47300099 |
| 19380 | 4:47488474 | 19388 | 4:47696820 | 19425 | 4:49010238 | 19454 | 4:49228470 | 19465 | 4:49442617 |
| 19505 | 4:50866615 | 19529 | 4:51642850 | 19540 | 4:52014153 | 19574 | 4:53063969 | 19623 | 4:54533598 |
| 19654 | 4:55652059 | 19655 | 4:55666248 | 19666 | 4:55979945 | 19678 | 4:56152616 | 19746 | 4:58369087 |
| 19753 | 4:58591231 | 19754 | 4:58596005 | 19769 | 4:59119437 | 19775 | 4:59214261 | 19791 | 4:59626845 |
| 19794 | 4:59754417 | 19815 | 4:60395032 | 19869 | 4:61855772 | 19915 | 4:63120245 | 19920 | 4:63231993 |
| 19939 | 4:63706634 | 19941 | 4:63737742 | 19965 | 4:64527362 | 19979 | 4:64927929 | 19981 | 4:64973879 |
| 20073 | 4:67329757 | 20080 | 4:67492078 | 20083 | 4:67548081 | 20121 | 4:68635250 | 20122 | 4:68672213 |
| 20195 | 4:70609606 | 20233 | 4:71767720 | 20235 | 4:71789741 | 20358 | 4:75159207 | 20362 | 4:75242210 |
| 20385 | 4:75797380 | 20413 | 4:76318250 | 20436 | 4:77029452 | 20481 | 4:77948146 | 20491 | 4:78185575 |
| 20539 | 4:79657303 | 20543 | 4:79839208 | 20568 | 4:80186925 | 20573 | 4:80245076 | 20587 | 4:80584868 |
| 20592 | 4:80719912 | 20661 | 4:82586451 | 20666 | 4:82698761 | 20712 | 4:83936797 | 20729 | 4:84451586 |
| 20765 | 4:85525303 | 20774 | 4:85682095 | 20781 | 4:85880701 | 20796 | 4:86159050 | 20846 | 4:87474051 |
| 20850 | 4:87586277 | 20873 | 4:88094522 | 20894 | 4:88685340 | 20914 | 4:89467898 | 20924 | 4:89712815 |
| 20974 | 4:90944945 | 21037 | 4:92282682 | 21054 | 4:92473823 | 21057 | 4:92523126 | 21063 | 4:92640436 |
| 21103 | 4:93420321 | 21105 | 4:93454704 | 21113 | 4:93574942 | 21118 | 4:93658997 | 21176 | 4:95336071 |
| 21177 | 4:95348378 | 21181 | 4:95493235 | 21227 | 4:96019036 | 21262 | 4:96809210 | 21279 | 4:96825379 |
| 21360 | 4:96942694 | 21366 | 4:97025110 | 21436 | 4:97663817 | 21456 | 4:98220002 | 21526 | 4:100120727 |
| 21537 | 4:100380798 | 21545 | 4:100648109 | 21550 | 4:100737518 | 21555 | 4:100801331 | 21644 | 4:102776227 |
| 21654 | 4:103076079 | 21677 | 4:103575595 | 21680 | 4:103670470 | 21746 | 4:105364910 | 21757 | 4:105516749 |
| 21761 | 4:105568960 | 21777 | 4:106062418 | 21819 | 4:106715919 | 21888 | 4:107648399 | 21906 | 4:108309499 |
| 21907 | 4:108319723 | 21926 | 4:108947713 | 21940 | 4:109363070 | 21947 | 4:109525615 | 21948 | 4:109528067 |
| 21949 | 4:109528137 | 21950 | 4:109529095 | 21951 | 4:109529531 | 21952 | 4:109529976 | 21953 | 4:109530257 |
| 21954 | 4:109530338 | 21955 | 4:109532512 | 21956 | 4:109533036 | 21957 | 4:109538711 | 21958 | 4:109538973 |
| 21959 | 4:109545654 | 21961 | 4:109576187 | 21985 | 4:110235765 | 21986 | 4:110257114 | 21999 | 4:110637707 |
| 22000 | 4:110672104 | 22001 | 4:110703849 | 22014 | 4:110890327 | 22025 | 4:111120368 | 22026 | 4:111120557 |
| 22061 | 4:111707261 | 22062 | 4:111708263 | 22063 | 4:111711546 | 22064 | 4:111711848 | 22065 | 4:111713444 |
| 22086 | 4:112129918 | 22089 | 4:112149705 | 22095 | 4:112210325 | 22107 | 4:112369141 | 22119 | 4:112555531 |
| 22179 | 4:113489475 | 22192 | 4:113598124 | 22193 | 4:113600602 | 22194 | 4:113601769 | 22196 | 4:113610307 |
| 22197 | 4:113612170 | 22198 | 4:113614202 | 22202 | 4:113636177 | 22203 | 4:113637533 | 22204 | 4:113639510 |
| 22205 | 4:113645913 | 22206 | 4:113646772 | 22207 | 4:113647228 | 22208 | 4:113647637 | 22209 | 4:113649086 |
| 22210 | 4:113650751 | 22211 | 4:113653050 | 22212 | 4:113670640 | 22213 | 4:113670882 | 22214 | 4:113671866 |
| 22215 | 4:113673914 | 22216 | 4:113673955 | 22217 | 4:113676621 | 22218 | 4:113680315 | 22351 | 4:115923041 |
| 22368 | 4:116278293 | 22377 | 4:116438965 | 22393 | 4:116746277 | 22401 | 4:116897937 | 22404 | 4:116926240 |
| 22405 | 4:116961074 | 22514 | 4:119167733 | 22520 | 4:119293731 | 22547 | 5:35727 | 22548 | 5:42965 |
| 22549 | 5:46543 | 22550 | 5:56066 | 22551 | 5:57672 | 22554 | 5:90000 | 22555 | 5:93744 |
| 22556 | 5:95203 | 22557 | 5:102783 | 22558 | 5:126748 | 22559 | 5:149452 | 22560 | 5:156011 |
| 22562 | 5:157715 | 22563 | 5:159858 | 22578 | 5:818560 | 22609 | 5:1354890 | 22616 | 5:1388630 |
| 22619 | 5:1458836 | 22642 | 5:1946239 | 22703 | 5:3492443 | 22727 | 5:4322265 | 22743 | 5:4570191 |
| 22752 | 5:4731333 | 22754 | 5:4732613 | 22775 | 5:5220278 | 22799 | 5:5717840 | 22851 | 5:6927428 |
| 22853 | 5:6949366 | 22892 | 5:7781205 | 22896 | 5:7895175 | 22903 | 5:8176883 | 22949 | 5:9380271 |
| 22955 | 5:9551052 | 22956 | 5:9555919 | 22960 | 5:9636042 | 22996 | 5:10313930 | 23013 | 5:10668196 |
| 23026 | 5:10924954 | 23029 | 5:10994036 | 23065 | 5:12039811 | 23100 | 5:12501985 | 23102 | 5:12524034 |
| 23125 | 5:13140668 | 23167 | 5:14461980 | 23181 | 5:14793206 | 23214 | 5:15447067 | 23218 | 5:15558924 |
| 23257 | 5:16336560 | 23268 | 5:16719670 | 23269 | 5:16739498 | 23279 | 5:17077362 | 23280 | 5:17078096 |
| 23292 | 5:17322229 | 23296 | 5:17492634 | 23298 | 5:17500602 | 23301 | 5:17665160 | 23307 | 5:17903579 |
| 23313 | 5:18155232 | 23326 | 5:18518099 | 23364 | 5:19695581 | 23400 | 5:20738200 | 23430 | 5:21391973 |
| 23432 | 5:21432976 | 23439 | 5:21643782 | 23440 | 5:21648427 | 23451 | 5:21871823 | 23459 | 5:21991889 |
| 23484 | 5:22479526 | 23485 | 5:22487130 | 23543 | 5:23622599 | 23555 | 5:23928759 | 23558 | 5:23956744 |
| 23613 | 5:25183734 | 23665 | 5:26532385 | 23677 | 5:26846005 | 23680 | 5:26972277 | 23681 | 5:26972772 |
| 23690 | 5:27127258 | 23702 | 5:27439061 | 23707 | 5:27544684 | 23733 | 5:27949133 | 23757 | 5:28498202 |
| 23799 | 5:29557818 | 23812 | 5:29809074 | 23822 | 5:29994317 | 23825 | 5:30019258 | 23946 | 5:33177185 |
| 23952 | 5:33331535 | 23953 | 5:33336405 | 23954 | 5:33337110 | 23968 | 5:33738237 | 24007 | 5:34880473 |
| 24042 | 5:35923865 | 24045 | 5:36012922 | 24048 | 5:36038265 | 24049 | 5:36042606 | 24058 | 5:36409810 |
| 24074 | 5:36939207 | 24120 | 5:37996697 | 24182 | 5:39657770 | 24190 | 5:39821210 | 24193 | 5:39927222 |
| 24238 | 5:41211155 | 24240 | 5:41232876 | 24241 | 5:41235105 | 24242 | 5:41236186 | 24258 | 5:41776747 |
| 24279 | 5:42522952 | 24322 | 5:43563557 | 24327 | 5:43634786 | 24366 | 5:44572552 | 24385 | 5:45181510 |
| 24386 | 5:45181566 | 24402 | 5:45615706 | 24417 | 5:45973787 | 24431 | 5:46166472 | 24441 | 5:46464570 |
| 24443 | 5:46517477 | 24452 | 5:46782312 | 24458 | 5:46936034 | 24459 | 5:46938241 | 24460 | 5:46938449 |
| 24461 | 5:46940754 | 24462 | 5:46941677 | 24475 | 5:47204347 | 24522 | 5:48389782 | 24529 | 5:48667259 |
| 24535 | 5:48760782 | 24540 | 5:48866791 | 24550 | 5:49202198 | 24559 | 5:49562957 | 24562 | 5:49625601 |
| 24570 | 5:49842789 | 24607 | 5:50836826 | 24779 | 5:55323383 | 24806 | 5:55945914 | 24858 | 5:57094271 |
| 24893 | 5:58015125 | 24920 | 5:59115316 | 24926 | 5:59337375 | 24942 | 5:59884753 | 24948 | 5:60008612 |
| 24991 | 5:60879895 | 24994 | 5:60984488 | 24999 | 5:61164840 | 25018 | 5:61884469 | 25034 | 5:62444037 |
| 25035 | 5:62445409 | 25036 | 5:62449226 | 25037 | 5:62449848 | 25038 | 5:62450932 | 25055 | 5:63033268 |
| 25082 | 5:63536637 | 25111 | 5:64402956 | 25124 | 5:64829651 | 25128 | 5:64926399 | 25129 | 5:64926523 |
| 25130 | 5:64928264 | 25164 | 5:65659261 | 25171 | 5:65755099 | 25197 | 5:66595784 | 25208 | 5:66799811 |
| 25219 | 5:67271370 | 25335 | 5:69618545 | 25426 | 5:71726794 | 25464 | 5:72378602 | 25466 | 5:72402551 |
| 25482 | 5:73010927 | 25486 | 5:73094311 | 25488 | 5:73189660 | 25550 | 5:74746217 | 25597 | 5:75667991 |
| 25606 | 5:75883986 | 25611 | 5:75946414 | 25665 | 5:77347106 | 25680 | 5:77945857 | 25725 | 5:78973946 |
| 25727 | 5:79017627 | 25735 | 5:79334960 | 25748 | 5:79714596 | 25749 | 5:79722250 | 25781 | 5:80877376 |
| 25791 | 5:81278052 | 25871 | 5:82792399 | 25884 | 5:83174887 | 25891 | 5:83348374 | 25900 | 5:83570417 |
| 25915 | 5:83863121 | 25918 | 5:83916047 | 25923 | 5:84010266 | 25926 | 5:84064602 | 25927 | 5:84064623 |
| 25928 | 5:84064772 | 25929 | 5:84065038 | 25930 | 5:84065171 | 25931 | 5:84065332 | 25932 | 5:84065339 |
| 25933 | 5:84065355 | 25934 | 5:84066088 | 25935 | 5:84066287 | 25936 | 5:84067645 | 25937 | 5:84068297 |
| 25938 | 5:84068606 | 25939 | 5:84068687 | 25940 | 5:84068732 | 25941 | 5:84068756 | 25942 | 5:84070079 |
| 25943 | 5:84070128 | 25944 | 5:84076319 | 25945 | 5:84076527 | 25946 | 5:84077100 | 25947 | 5:84080168 |
| 25949 | 5:84086306 | 25950 | 5:84086861 | 25951 | 5:84090372 | 25952 | 5:84090487 | 25953 | 5:84097284 |
| 25954 | 5:84097885 | 25955 | 5:84097985 | 25956 | 5:84099214 | 25957 | 5:84100319 | 25958 | 5:84101337 |
| 25959 | 5:84101438 | 25960 | 5:84101462 | 25961 | 5:84101494 | 25962 | 5:84101519 | 25963 | 5:84101555 |
| 25964 | 5:84101619 | 25965 | 5:84101701 | 25966 | 5:84102206 | 25967 | 5:84102236 | 25969 | 5:84103792 |
| 25970 | 5:84103930 | 25971 | 5:84104200 | 25972 | 5:84107013 | 25974 | 5:84115513 | 25975 | 5:84115550 |
| 25976 | 5:84115605 | 25977 | 5:84115682 | 25978 | 5:84115698 | 25979 | 5:84115763 | 25980 | 5:84115908 |
| 25981 | 5:84116028 | 25982 | 5:84116506 | 25983 | 5:84116583 | 25984 | 5:84119378 | 25985 | 5:84119408 |
| 25986 | 5:84119435 | 25987 | 5:84120471 | 25988 | 5:84120477 | 25989 | 5:84120540 | 25990 | 5:84120562 |
| 25991 | 5:84120612 | 25992 | 5:84120761 | 25993 | 5:84120772 | 25994 | 5:84120800 | 25995 | 5:84120828 |
| 25996 | 5:84120900 | 25997 | 5:84120980 | 25998 | 5:84120987 | 25999 | 5:84121087 | 26000 | 5:84121318 |
| 26001 | 5:84122651 | 26002 | 5:84122709 | 26003 | 5:84122905 | 26004 | 5:84123385 | 26005 | 5:84123428 |
| 26006 | 5:84124508 | 26007 | 5:84124539 | 26008 | 5:84124557 | 26009 | 5:84124558 | 26010 | 5:84124682 |
| 26011 | 5:84125648 | 26012 | 5:84125855 | 26013 | 5:84126181 | 26014 | 5:84126183 | 26015 | 5:84126222 |
| 26016 | 5:84126766 | 26017 | 5:84126941 | 26018 | 5:84126965 | 26019 | 5:84126986 | 26020 | 5:84127998 |
| 26021 | 5:84128048 | 26022 | 5:84128850 | 26023 | 5:84128968 | 26024 | 5:84129307 | 26025 | 5:84129349 |
| 26026 | 5:84129709 | 26027 | 5:84130342 | 26028 | 5:84130419 | 26029 | 5:84130836 | 26030 | 5:84130988 |
| 26031 | 5:84131404 | 26032 | 5:84131699 | 26033 | 5:84131703 | 26034 | 5:84131742 | 26035 | 5:84132713 |
| 26036 | 5:84132801 | 26037 | 5:84132954 | 26038 | 5:84133129 | 26039 | 5:84133144 | 26040 | 5:84133170 |
| 26041 | 5:84133302 | 26042 | 5:84133384 | 26043 | 5:84133400 | 26044 | 5:84133418 | 26045 | 5:84135047 |
| 26046 | 5:84135179 | 26047 | 5:84135274 | 26048 | 5:84137923 | 26049 | 5:84138207 | 26050 | 5:84138277 |
| 26051 | 5:84138403 | 26052 | 5:84138617 | 26053 | 5:84138641 | 26054 | 5:84138927 | 26055 | 5:84142947 |
| 26056 | 5:84143312 | 26069 | 5:84405330 | 26105 | 5:85329574 | 26123 | 5:85783695 | 26129 | 5:85892084 |
| 26147 | 5:86451423 | 26153 | 5:86534299 | 26158 | 5:86590201 | 26161 | 5:86629956 | 26175 | 5:86950590 |
| 26207 | 5:87624956 | 26209 | 5:87645403 | 26219 | 5:87859562 | 26287 | 5:88745049 | 26319 | 5:89399078 |
| 26320 | 5:89427836 | 26334 | 5:89821006 | 26385 | 5:91059021 | 26400 | 5:91433996 | 26401 | 5:91479219 |
| 26434 | 5:92344711 | 26436 | 5:92351975 | 26437 | 5:92360410 | 26440 | 5:92389962 | 26441 | 5:92399584 |
| 26442 | 5:92402496 | 26443 | 5:92402509 | 26444 | 5:92407767 | 26445 | 5:92412423 | 26446 | 5:92412426 |
| 26447 | 5:92412918 | 26448 | 5:92415826 | 26449 | 5:92416355 | 26450 | 5:92418513 | 26451 | 5:92422512 |
| 26452 | 5:92422802 | 26453 | 5:92423893 | 26454 | 5:92426805 | 26455 | 5:92428903 | 26456 | 5:92428912 |
| 26457 | 5:92429850 | 26458 | 5:92429875 | 26459 | 5:92430956 | 26460 | 5:92431470 | 26461 | 5:92431717 |
| 26462 | 5:92431719 | 26463 | 5:92432015 | 26464 | 5:92432232 | 26465 | 5:92432425 | 26466 | 5:92432633 |
| 26472 | 5:92541180 | 26487 | 5:92897747 | 26582 | 5:95173664 | 26584 | 5:95197902 | 26656 | 5:96676782 |
| 26698 | 5:97374091 | 26709 | 5:97622463 | 26728 | 5:98063850 | 26729 | 5:98064276 | 26735 | 5:98193113 |
| 26736 | 5:98202197 | 26738 | 5:98236533 | 26768 | 5:98748721 | 26772 | 5:98826571 | 26828 | 5:99948490 |
| 26861 | 5:100535488 | 26863 | 5:100631069 | 26870 | 5:100703214 | 26884 | 5:100953490 | 26893 | 5:101083268 |
| 26894 | 5:101089292 | 26899 | 5:101221558 | 26914 | 5:101607756 | 26921 | 5:101673726 | 26928 | 5:101914630 |
| 26940 | 5:102460767 | 26943 | 5:102476964 | 26968 | 5:103154349 | 26986 | 5:103552417 | 27001 | 5:103882706 |
| 27005 | 5:103981305 | 27068 | 5:105100005 | 27114 | 5:105867389 | 27140 | 5:106221085 | 27148 | 5:106293076 |
| 27162 | 5:106480592 | 27188 | 5:106887255 | 27202 | 5:107008747 | 27222 | 5:107342588 | 27232 | 5:107495491 |
| 27234 | 5:107522927 | 27269 | 5:108224683 | 27274 | 5:108258258 | 27280 | 5:108472906 | 27281 | 5:108473260 |
| 27282 | 5:108476147 | 27283 | 5:108478132 | 27284 | 5:108485933 | 27285 | 5:108486228 | 27286 | 5:108486756 |
| 27287 | 5:108487832 | 27288 | 5:108489677 | 27289 | 5:108490693 | 27290 | 5:108490734 | 27295 | 5:108546887 |
| 27297 | 5:108575310 | 27399 | 5:110511118 | 27440 | 5:111144555 | 27462 | 5:111761777 | 27467 | 5:111860816 |
| 27481 | 5:112115946 | 27519 | 5:112982896 | 27525 | 5:113133130 | 27539 | 5:113320727 | 27547 | 5:113650431 |
| 27566 | 5:114143008 | 27569 | 5:114206160 | 27572 | 5:114283857 | 27580 | 5:114719162 | 27583 | 5:114775926 |
| 27605 | 5:115427295 | 27652 | 5:116126971 | 27656 | 5:116179833 | 27676 | 5:116586289 | 27677 | 5:116588965 |
| 27678 | 5:116590488 | 27679 | 5:116590506 | 27680 | 5:116590745 | 27682 | 5:116592015 | 27683 | 5:116592619 |
| 27684 | 5:116592903 | 27687 | 5:116593192 | 27688 | 5:116593569 | 27694 | 5:116594398 | 27695 | 5:116594541 |
| 27696 | 5:116594546 | 27697 | 5:116594548 | 27698 | 5:116594847 | 27699 | 5:116595244 | 27700 | 5:116595727 |
| 27701 | 5:116595799 | 27702 | 5:116596186 | 27704 | 5:116596441 | 27705 | 5:116596625 | 27706 | 5:116596745 |
| 27707 | 5:116596913 | 27708 | 5:116597118 | 27711 | 5:116598702 | 27712 | 5:116598835 | 27715 | 5:116602527 |
| 27721 | 5:116677042 | 27731 | 5:116722878 | 27808 | 5:117709009 | 27809 | 5:117711361 | 27813 | 5:117773986 |
| 27826 | 5:117998759 | 27831 | 5:118098871 | 27838 | 5:118250217 | 27860 | 5:118703768 | 27866 | 5:118745750 |
| 27903 | 5:119271970 | 27908 | 5:119361103 | 27916 | 5:119587043 | 27948 | 6:528320 | 27970 | 6:993117 |
| 27999 | 6:1950764 | 28000 | 6:1951302 | 28001 | 6:1951498 | 28002 | 6:1951714 | 28003 | 6:1951719 |
| 28004 | 6:1951800 | 28005 | 6:1951981 | 28006 | 6:1952392 | 28007 | 6:1955233 | 28008 | 6:1955330 |
| 28009 | 6:1955401 | 28010 | 6:1955549 | 28011 | 6:1955698 | 28012 | 6:1956232 | 28013 | 6:1956278 |
| 28014 | 6:1957428 | 28015 | 6:1957620 | 28016 | 6:1957800 | 28017 | 6:1957812 | 28018 | 6:1957895 |
| 28019 | 6:1958152 | 28020 | 6:1962365 | 28021 | 6:1963113 | 28022 | 6:1963759 | 28023 | 6:1963825 |
| 28024 | 6:1963953 | 28026 | 6:1973036 | 28027 | 6:1974486 | 28028 | 6:1974895 | 28061 | 6:2801468 |
| 28063 | 6:2860306 | 28064 | 6:2862765 | 28072 | 6:3067202 | 28074 | 6:3073689 | 28075 | 6:3084367 |
| 28129 | 6:3786375 | 28155 | 6:4418616 | 28168 | 6:4782253 | 28170 | 6:4824255 | 28221 | 6:6133626 |
| 28238 | 6:6309983 | 28278 | 6:7061434 | 28286 | 6:7250536 | 28293 | 6:7394514 | 28319 | 6:8317563 |
| 28336 | 6:8929534 | 28337 | 6:8944681 | 28339 | 6:8955494 | 28341 | 6:8960665 | 28343 | 6:8972751 |
| 28345 | 6:8980990 | 28346 | 6:8985187 | 28347 | 6:8989449 | 28352 | 6:9079158 | 28353 | 6:9081859 |
| 28354 | 6:9085203 | 28356 | 6:9094449 | 28373 | 6:9291690 | 28377 | 6:9465992 | 28389 | 6:9964731 |
| 28390 | 6:9965161 | 28391 | 6:9976901 | 28393 | 6:9992803 | 28395 | 6:10029462 | 28409 | 6:10453731 |
| 28422 | 6:10849905 | 28435 | 6:11149738 | 28440 | 6:11315175 | 28605 | 6:15051875 | 28641 | 6:15680456 |
| 28686 | 6:16603866 | 28718 | 6:17241712 | 28757 | 6:18035615 | 28758 | 6:18035662 | 28759 | 6:18035884 |
| 28760 | 6:18036845 | 28766 | 6:18057767 | 28795 | 6:18806853 | 28819 | 6:19264271 | 28820 | 6:19264527 |
| 28852 | 6:19965110 | 28878 | 6:20517010 | 28888 | 6:20729903 | 28944 | 6:22097118 | 28970 | 6:22554116 |
| 29044 | 6:24253816 | 29057 | 6:24617112 | 29061 | 6:24750491 | 29069 | 6:24940966 | 29114 | 6:25994106 |
| 29115 | 6:25995000 | 29123 | 6:26081649 | 29151 | 6:26895539 | 29159 | 6:27119474 | 29179 | 6:27632577 |
| 29182 | 6:27765059 | 29183 | 6:27794604 | 29196 | 6:28156905 | 29204 | 6:28392137 | 29233 | 6:29155222 |
| 29374 | 6:30901671 | 29490 | 6:34238062 | 29722 | 6:34572571 | 29939 | 6:35180069 | 29996 | 6:35924006 |
| 29997 | 6:35924204 | 29998 | 6:35925489 | 29999 | 6:35931470 | 30000 | 6:35932579 | 30010 | 6:35978601 |
| 30133 | 6:36426239 | 30136 | 6:36429755 | 30138 | 6:36430937 | 30160 | 6:36467397 | 30164 | 6:36475104 |
| 30165 | 6:36477167 | 30168 | 6:36481433 | 30169 | 6:36481434 | 30171 | 6:36483188 | 30174 | 6:36489959 |
| 30175 | 6:36490754 | 30234 | 6:36545679 | 30259 | 6:36568156 | 30261 | 6:36568846 | 30328 | 6:36807893 |
| 30329 | 6:36808225 | 30459 | 6:37029628 | 30462 | 6:37032995 | 30468 | 6:37035541 | 30566 | 6:37095299 |
| 30653 | 6:37151905 | 30655 | 6:37152373 | 30679 | 6:37160005 | 30735 | 6:37172974 | 30736 | 6:37173048 |
| 30748 | 6:37175662 | 30750 | 6:37175773 | 30757 | 6:37176677 | 30772 | 6:37181087 | 30781 | 6:37182773 |
| 30787 | 6:37183901 | 30792 | 6:37189736 | 30794 | 6:37192102 | 30802 | 6:37214389 | 30852 | 6:37251132 |
| 30858 | 6:37262825 | 30921 | 6:37299855 | 31027 | 6:37329948 | 31093 | 6:37365265 | 31180 | 6:37431101 |
| 31284 | 6:37636883 | 31285 | 6:37636900 | 31524 | 6:37936087 | 31846 | 6:38545113 | 31894 | 6:38558946 |
| 31930 | 6:38800398 | 31931 | 6:38830763 | 31935 | 6:38855286 | 31936 | 6:38862311 | 31937 | 6:38862987 |
| 31938 | 6:38875748 | 31940 | 6:38879318 | 31941 | 6:38882135 | 31980 | 6:39565429 | 31983 | 6:39593110 |
| 32001 | 6:39758696 | 32010 | 6:39853149 | 32018 | 6:40036007 | 32020 | 6:40036943 | 32022 | 6:40040343 |
| 32023 | 6:40053452 | 32025 | 6:40058671 | 32030 | 6:40130949 | 32056 | 6:40611279 | 32063 | 6:40707783 |
| 32064 | 6:40708542 | 32065 | 6:40708988 | 32066 | 6:40709098 | 32067 | 6:40709130 | 32068 | 6:40709457 |
| 32069 | 6:40709727 | 32070 | 6:40709846 | 32071 | 6:40711310 | 32072 | 6:40711346 | 32073 | 6:40711409 |
| 32074 | 6:40711422 | 32075 | 6:40711439 | 32076 | 6:40713027 | 32077 | 6:40713351 | 32078 | 6:40713605 |
| 32079 | 6:40713909 | 32080 | 6:40714153 | 32081 | 6:40714938 | 32082 | 6:40715195 | 32083 | 6:40716811 |
| 32084 | 6:40721324 | 32087 | 6:40743678 | 32090 | 6:40770454 | 32091 | 6:40771992 | 32092 | 6:40771993 |
| 32186 | 6:41827472 | 32240 | 6:42833384 | 32249 | 6:42951534 | 32250 | 6:42951582 | 32251 | 6:42951695 |
| 32252 | 6:42951801 | 32253 | 6:42952447 | 32254 | 6:42953604 | 32255 | 6:42954090 | 32256 | 6:42956624 |
| 32257 | 6:42957873 | 32258 | 6:42961867 | 32259 | 6:42963490 | 32260 | 6:42966370 | 32261 | 6:42969572 |
| 32265 | 6:42996149 | 32268 | 6:43024569 | 32269 | 6:43035223 | 32270 | 6:43035520 | 32278 | 6:43178587 |
| 32292 | 6:43450785 | 32322 | 6:43878243 | 32328 | 6:43959607 | 32364 | 6:44686809 | 32417 | 6:44828139 |
| 32510 | 6:45452605 | 32511 | 6:45453963 | 32521 | 6:45702584 | 32555 | 6:46424441 | 32556 | 6:46446420 |
| 32564 | 6:46615384 | 32573 | 6:46883981 | 32575 | 6:46926732 | 32584 | 6:47108047 | 32611 | 6:47785922 |
| 32645 | 6:48798347 | 32726 | 6:50704099 | 32732 | 6:50885777 | 32742 | 6:51188886 | 32764 | 6:51982250 |
| 32769 | 6:52083644 | 32804 | 6:52427412 | 32805 | 6:52431484 | 32806 | 6:52434675 | 32807 | 6:52444382 |
| 32808 | 6:52444842 | 32809 | 6:52451412 | 32819 | 6:52675393 | 32832 | 6:53029143 | 32838 | 6:53151451 |
| 32907 | 6:55106382 | 32908 | 6:55121620 | 32912 | 6:55288741 | 32916 | 6:55337592 | 32921 | 6:55478053 |
| 32922 | 6:55478241 | 32923 | 6:55480385 | 32924 | 6:55481575 | 32925 | 6:55483221 | 32926 | 6:55483692 |
| 32927 | 6:55484196 | 32928 | 6:55484209 | 32929 | 6:55484776 | 32930 | 6:55484984 | 32931 | 6:55484996 |
| 32932 | 6:55485652 | 32933 | 6:55487306 | 32934 | 6:55487659 | 32935 | 6:55487698 | 32936 | 6:55487961 |
| 32937 | 6:55488137 | 32938 | 6:55488439 | 32939 | 6:55498328 | 32945 | 6:55534953 | 33154 | 6:60793645 |
| 33178 | 6:61401990 | 33201 | 6:62075988 | 33220 | 6:62746494 | 33287 | 6:64826176 | 33288 | 6:64860935 |
| 33301 | 6:65368807 | 33320 | 6:65902330 | 33385 | 6:68144658 | 33560 | 6:73271530 | 33568 | 6:73524311 |
| 33587 | 6:74274673 | 33588 | 6:74278129 | 33590 | 6:74325296 | 33595 | 6:74538501 | 33604 | 6:74934981 |
| 33638 | 6:76140833 | 33664 | 6:76838570 | 33669 | 6:76918617 | 33761 | 6:80397556 | 33786 | 6:80984642 |
| 33856 | 6:83284076 | 33857 | 6:83290251 | 33889 | 6:83810111 | 33919 | 6:84577409 | 33945 | 6:85170579 |
| 33958 | 6:85452102 | 33960 | 6:85473180 | 33973 | 6:85798206 | 33990 | 6:86146685 | 34015 | 6:86736214 |
| 34039 | 6:87076207 | 34076 | 6:87751257 | 34095 | 6:88150285 | 34146 | 6:89563672 | 34160 | 6:89750491 |
| 34167 | 6:89906046 | 34223 | 6:90999178 | 34252 | 6:91356781 | 34256 | 6:91397116 | 34311 | 6:92717918 |
| 34325 | 6:93027241 | 34355 | 6:93561742 | 34379 | 6:94081466 | 34380 | 6:94087064 | 34382 | 6:94089501 |
| 34383 | 6:94094573 | 34384 | 6:94096218 | 34385 | 6:94098014 | 34386 | 6:94098380 | 34387 | 6:94098998 |
| 34456 | 6:96190398 | 34499 | 6:97061854 | 34533 | 6:97481556 | 34534 | 6:97481830 | 34535 | 6:97482142 |
| 34536 | 6:97486160 | 34588 | 6:98499066 | 34597 | 6:98688262 | 34647 | 6:100180448 | 34648 | 6:100181539 |
| 34649 | 6:100181706 | 34650 | 6:100181746 | 34651 | 6:100182503 | 34652 | 6:100183086 | 34653 | 6:100183464 |
| 34654 | 6:100185002 | 34655 | 6:100185489 | 34656 | 6:100185949 | 34657 | 6:100186040 | 34658 | 6:100186295 |
| 34673 | 6:100520648 | 34698 | 6:101088719 | 34699 | 6:101089428 | 34765 | 6:102387484 | 34775 | 6:102527385 |
| 34820 | 6:103206312 | 34874 | 6:104209750 | 34913 | 6:105016409 | 34983 | 6:106924221 | 34985 | 6:106930321 |
| 34998 | 6:107232252 | 35017 | 6:107833335 | 35031 | 6:108314278 | 35032 | 6:108316551 | 35035 | 6:108336234 |
| 35040 | 6:108483986 | 35074 | 6:109282710 | 35086 | 6:109549186 | 35112 | 6:110036778 | 35120 | 6:110267946 |
| 35121 | 6:110296111 | 35154 | 6:111027360 | 35170 | 6:111251001 | 35181 | 6:111506757 | 35194 | 6:111752185 |
| 35195 | 6:111753497 | 35196 | 6:111755682 | 35203 | 6:111845284 | 35215 | 6:112274283 | 35225 | 6:112517961 |
| 35254 | 6:112951648 | 35258 | 6:113193897 | 35264 | 6:113278790 | 35269 | 6:113338198 | 35302 | 6:113832391 |
| 35304 | 6:113899018 | 35316 | 6:114099166 | 35317 | 6:114104514 | 35338 | 6:114493111 | 35356 | 6:114802066 |
| 35377 | 6:115232986 | 35397 | 6:115802755 | 35522 | 7:1173379 | 35548 | 7:1688750 | 35608 | 7:2637204 |
| 35647 | 7:3292376 | 35723 | 7:4848657 | 35745 | 7:5160131 | 35746 | 7:5169066 | 35754 | 7:5443639 |
| 35777 | 7:5627311 | 35779 | 7:5637555 | 35814 | 7:6109291 | 35815 | 7:6109369 | 35816 | 7:6110105 |
| 35817 | 7:6110223 | 35818 | 7:6110813 | 35819 | 7:6112482 | 35820 | 7:6112502 | 35821 | 7:6112503 |
| 35822 | 7:6112832 | 35824 | 7:6114838 | 35825 | 7:6118415 | 35826 | 7:6118766 | 35827 | 7:6119488 |
| 35828 | 7:6122989 | 35829 | 7:6123149 | 35830 | 7:6123438 | 35831 | 7:6128021 | 35832 | 7:6130590 |
| 35833 | 7:6130687 | 35834 | 7:6131292 | 35835 | 7:6131297 | 35836 | 7:6132484 | 35837 | 7:6132747 |
| 35838 | 7:6133083 | 35840 | 7:6137471 | 35854 | 7:6339944 | 35855 | 7:6341414 | 35856 | 7:6341470 |
| 35857 | 7:6341501 | 35858 | 7:6345398 | 35860 | 7:6348000 | 35861 | 7:6348353 | 35862 | 7:6352211 |
| 35863 | 7:6356100 | 35864 | 7:6356608 | 35865 | 7:6356670 | 35881 | 7:6710471 | 35894 | 7:6824241 |
| 35903 | 7:6999017 | 35914 | 7:7143257 | 35929 | 7:7557725 | 35930 | 7:7562490 | 35933 | 7:7584113 |
| 35959 | 7:8549050 | 35966 | 7:8914661 | 35977 | 7:9686485 | 35978 | 7:9760724 | 36006 | 7:11067502 |
| 36013 | 7:11123621 | 36031 | 7:11430993 | 36036 | 7:11668056 | 36046 | 7:11952515 | 36081 | 7:12499238 |
| 36157 | 7:13717835 | 36185 | 7:14182114 | 36243 | 7:15057296 | 36303 | 7:16284081 | 36325 | 7:16641516 |
| 36363 | 7:17636616 | 36365 | 7:17641071 | 36384 | 7:18214147 | 36409 | 7:18665015 | 36416 | 7:18849856 |
| 36433 | 7:19227351 | 36442 | 7:19317690 | 36444 | 7:19333133 | 36461 | 7:19721740 | 36516 | 7:20334637 |
| 36524 | 7:20395009 | 36536 | 7:20701671 | 36537 | 7:20704005 | 36539 | 7:20747147 | 36548 | 7:20935768 |
| 36639 | 7:23007875 | 36650 | 7:23297694 | 36651 | 7:23350564 | 36657 | 7:23528238 | 36658 | 7:23528885 |
| 36684 | 7:24067558 | 36692 | 7:24236323 | 36709 | 7:24746921 | 36734 | 7:25342888 | 36737 | 7:25403017 |
| 36843 | 7:27517669 | 36845 | 7:27577715 | 36846 | 7:27588151 | 36892 | 7:28685778 | 36910 | 7:29075991 |
| 36945 | 7:29836572 | 36952 | 7:29934618 | 36955 | 7:30045346 | 36969 | 7:30374597 | 36979 | 7:30662217 |
| 37046 | 7:31780219 | 37055 | 7:32039528 | 37090 | 7:33006143 | 37103 | 7:33314191 | 37140 | 7:34167737 |
| 37157 | 7:34561935 | 37166 | 7:34800911 | 37204 | 7:35829441 | 37224 | 7:36375051 | 37225 | 7:36381643 |
| 37271 | 7:37512158 | 37358 | 7:39431184 | 37383 | 7:40018615 | 37386 | 7:40105218 | 37387 | 7:40106188 |
| 37403 | 7:40313129 | 37421 | 7:40543374 | 37501 | 7:42717113 | 37502 | 7:42793836 | 37562 | 7:44383968 |
| 37577 | 7:44807464 | 37579 | 7:44874918 | 37589 | 7:45086131 | 37597 | 7:45264623 | 37598 | 7:45268714 |
| 37606 | 7:45462352 | 37628 | 7:46197379 | 37629 | 7:46207779 | 37647 | 7:46698742 | 37652 | 7:46763773 |
| 37665 | 7:47007606 | 37666 | 7:47009580 | 37667 | 7:47010093 | 37668 | 7:47010163 | 37669 | 7:47011323 |
| 37670 | 7:47011475 | 37671 | 7:47012085 | 37672 | 7:47012699 | 37674 | 7:47017310 | 37675 | 7:47018943 |
| 37676 | 7:47021031 | 37716 | 7:48370104 | 37736 | 7:48785206 | 37758 | 7:49338040 | 37807 | 7:51678528 |
| 37832 | 7:52217990 | 37892 | 7:52801631 | 37901 | 7:53051717 | 37903 | 7:53072922 | 37907 | 7:53192775 |
| 37929 | 7:53916384 | 37998 | 7:55634474 | 38004 | 7:55875367 | 38018 | 7:56132046 | 38036 | 7:56553298 |
| 38044 | 7:56873627 | 38072 | 7:57843543 | 38089 | 7:58267508 | 38091 | 7:58338126 | 38139 | 7:59331366 |
| 38146 | 7:59368692 | 38166 | 7:59579502 | 38249 | 7:61303310 | 38261 | 7:61485578 | 38264 | 7:61501849 |
| 38305 | 7:62532423 | 38306 | 7:62554501 | 38309 | 7:62558404 | 38377 | 7:63949067 | 38388 | 7:64285750 |
| 38397 | 7:64625175 | 38412 | 7:64930029 | 38413 | 7:64937658 | 38420 | 7:65072560 | 38432 | 7:65258702 |
| 38507 | 7:66460989 | 38538 | 7:67323191 | 38555 | 7:67781532 | 38557 | 7:67828291 | 38563 | 7:67940525 |
| 38586 | 7:68625153 | 38589 | 7:68641820 | 38642 | 7:69866274 | 38644 | 7:69879505 | 38646 | 7:69905767 |
| 38653 | 7:70037542 | 38655 | 7:70093469 | 38656 | 7:70099250 | 38666 | 7:70331724 | 38669 | 7:70419871 |
| 38689 | 7:71130508 | 38698 | 7:71415976 | 38716 | 7:71712692 | 38717 | 7:71727860 | 38722 | 7:71825182 |
| 38723 | 7:71830368 | 38725 | 7:71921897 | 38754 | 7:72753552 | 38794 | 7:74007645 | 38826 | 7:74850810 |
| 38869 | 7:75797751 | 38879 | 7:76006096 | 38880 | 7:76006417 | 38881 | 7:76010723 | 38916 | 7:76940482 |
| 38930 | 7:77150855 | 38941 | 7:77453724 | 38950 | 7:77656774 | 38964 | 7:77949425 | 38989 | 7:78379223 |
| 39000 | 7:78748906 | 39008 | 7:79016201 | 39009 | 7:79016267 | 39010 | 7:79016787 | 39011 | 7:79017056 |
| 39012 | 7:79017378 | 39013 | 7:79017385 | 39014 | 7:79017772 | 39015 | 7:79017959 | 39016 | 7:79018035 |
| 39017 | 7:79018571 | 39018 | 7:79018616 | 39019 | 7:79018620 | 39020 | 7:79019988 | 39021 | 7:79023682 |
| 39022 | 7:79025120 | 39023 | 7:79025266 | 39024 | 7:79041811 | 39025 | 7:79042476 | 39027 | 7:79048979 |
| 39028 | 7:79070660 | 39029 | 7:79071063 | 39031 | 7:79135260 | 39032 | 7:79135270 | 39040 | 7:79367716 |
| 39056 | 7:79660008 | 39076 | 7:80098520 | 39077 | 7:80104490 | 39096 | 7:80463366 | 39111 | 7:80573664 |
| 39122 | 7:80835359 | 39133 | 7:81097721 | 39221 | 7:82912666 | 39241 | 7:83502868 | 39259 | 7:83929718 |
| 39299 | 7:85098423 | 39310 | 7:85314414 | 39315 | 7:85586461 | 39383 | 7:87496719 | 39413 | 7:88462551 |
| 39425 | 7:88705722 | 39622 | 7:91895030 | 39706 | 7:94524953 | 39712 | 7:94671798 | 39713 | 7:94672003 |
| 39714 | 7:94688436 | 39715 | 7:94691325 | 39728 | 7:95018455 | 39768 | 7:95975870 | 39773 | 7:96088093 |
| 39774 | 7:96093182 | 39795 | 7:96175080 | 39796 | 7:96175132 | 39797 | 7:96175168 | 39798 | 7:96175341 |
| 39799 | 7:96175357 | 39800 | 7:96175409 | 39801 | 7:96175451 | 39802 | 7:96175628 | 39803 | 7:96176143 |
| 39804 | 7:96176528 | 39805 | 7:96176908 | 39809 | 7:96193901 | 39812 | 7:96199226 | 39815 | 7:96205718 |
| 39816 | 7:96205942 | 39817 | 7:96206884 | 39818 | 7:96207458 | 39819 | 7:96207882 | 39820 | 7:96208060 |
| 39821 | 7:96208106 | 39822 | 7:96208582 | 39823 | 7:96208995 | 39824 | 7:96209096 | 39825 | 7:96209862 |
| 39826 | 7:96211592 | 39827 | 7:96211829 | 39828 | 7:96216996 | 39832 | 7:96221555 | 39833 | 7:96223919 |
| 39834 | 7:96228261 | 39835 | 7:96228951 | 39836 | 7:96231447 | 39837 | 7:96235988 | 39838 | 7:96236371 |
| 39839 | 7:96236926 | 39840 | 7:96239015 | 39841 | 7:96239023 | 39842 | 7:96242837 | 39843 | 7:96246776 |
| 39844 | 7:96246937 | 39845 | 7:96248619 | 39846 | 7:96248835 | 39847 | 7:96249753 | 39848 | 7:96249787 |
| 39849 | 7:96249862 | 39850 | 7:96254816 | 39851 | 7:96263665 | 39852 | 7:96264122 | 39853 | 7:96264141 |
| 39854 | 7:96265124 | 39855 | 7:96265144 | 39856 | 7:96265161 | 39857 | 7:96265371 | 39858 | 7:96265379 |
| 39860 | 7:96271576 | 39861 | 7:96271653 | 39862 | 7:96273181 | 39863 | 7:96273246 | 39864 | 7:96273247 |
| 39865 | 7:96273562 | 39866 | 7:96273613 | 39867 | 7:96274725 | 39869 | 7:96291284 | 39870 | 7:96291379 |
| 39871 | 7:96291389 | 39872 | 7:96291500 | 39873 | 7:96291530 | 39874 | 7:96293330 | 39875 | 7:96293559 |
| 39876 | 7:96293758 | 39877 | 7:96293861 | 39878 | 7:96295220 | 39879 | 7:96295518 | 39880 | 7:96295778 |
| 39881 | 7:96295878 | 39882 | 7:96295906 | 39883 | 7:96295937 | 39884 | 7:96296075 | 39885 | 7:96296087 |
| 39886 | 7:96296169 | 39887 | 7:96296233 | 39888 | 7:96296513 | 39889 | 7:96296531 | 39890 | 7:96296546 |
| 39891 | 7:96296683 | 39892 | 7:96297463 | 39893 | 7:96297555 | 39894 | 7:96297857 | 39895 | 7:96298677 |
| 39896 | 7:96299647 | 39897 | 7:96300501 | 39898 | 7:96303921 | 39899 | 7:96304204 | 39900 | 7:96305341 |
| 39901 | 7:96305698 | 39902 | 7:96307964 | 39903 | 7:96308750 | 39904 | 7:96308945 | 39905 | 7:96309033 |
| 39906 | 7:96310180 | 39907 | 7:96311062 | 39909 | 7:96314531 | 39910 | 7:96314540 | 39913 | 7:96318724 |
| 39914 | 7:96322543 | 39915 | 7:96322732 | 39916 | 7:96323877 | 39917 | 7:96323928 | 39918 | 7:96324360 |
| 39919 | 7:96330432 | 39920 | 7:96331236 | 39921 | 7:96331603 | 39922 | 7:96333195 | 39923 | 7:96333277 |
| 39924 | 7:96333341 | 39925 | 7:96333563 | 39926 | 7:96333575 | 39927 | 7:96334610 | 39928 | 7:96334702 |
| 39929 | 7:96334763 | 39930 | 7:96335706 | 39931 | 7:96337614 | 39933 | 7:96354116 | 39935 | 7:96355876 |
| 39936 | 7:96358329 | 39937 | 7:96361391 | 39938 | 7:96363115 | 39940 | 7:96374561 | 39949 | 7:96519372 |
| 39953 | 7:96606759 | 40022 | 7:98399080 | 40043 | 7:99244131 | 40046 | 7:99254092 | 40085 | 7:100105110 |
| 40106 | 7:100563689 | 40108 | 7:100578900 | 40112 | 7:100659403 | 40127 | 7:101215525 | 40166 | 7:102357637 |
| 40201 | 7:103052550 | 40216 | 7:103329082 | 40224 | 7:103487103 | 40229 | 7:103675594 | 40232 | 7:103729325 |
| 40239 | 7:103982179 | 40240 | 7:103982180 | 40241 | 7:103982288 | 40242 | 7:103982423 | 40243 | 7:103985377 |
| 40253 | 7:104230973 | 40291 | 7:105373160 | 40294 | 7:105399311 | 40296 | 7:105442182 | 40297 | 7:105515412 |
| 40324 | 7:106146247 | 40372 | 7:107094209 | 40415 | 7:108495274 | 40496 | 8:356371 | 40500 | 8:395127 |
| 40585 | 8:2113854 | 40598 | 8:2387478 | 40600 | 8:2420577 | 40637 | 8:3508161 | 40640 | 8:3527178 |
| 40645 | 8:3640550 | 40647 | 8:3666778 | 40718 | 8:5496138 | 40732 | 8:5839173 | 40736 | 8:5906023 |
| 40751 | 8:6327904 | 40804 | 8:7593827 | 40861 | 8:8914108 | 40865 | 8:9015228 | 40882 | 8:9392626 |
| 40960 | 8:11244122 | 40966 | 8:11330242 | 40968 | 8:11403633 | 40975 | 8:11522513 | 40984 | 8:11668581 |
| 40991 | 8:11856184 | 40997 | 8:12018134 | 41009 | 8:12363854 | 41050 | 8:13811430 | 41095 | 8:15117985 |
| 41099 | 8:15188506 | 41110 | 8:15477698 | 41112 | 8:15494200 | 41118 | 8:15553158 | 41119 | 8:15574036 |
| 41126 | 8:15701758 | 41137 | 8:16168316 | 41138 | 8:16168775 | 41201 | 8:17444173 | 41211 | 8:17638060 |
| 41227 | 8:18138392 | 41250 | 8:18931382 | 41279 | 8:19741400 | 41290 | 8:19953078 | 41331 | 8:20945241 |
| 41343 | 8:21351226 | 41403 | 8:23473039 | 41410 | 8:23722262 | 41451 | 8:24658012 | 41460 | 8:24730121 |
| 41567 | 8:25718774 | 41578 | 8:25952516 | 41581 | 8:26020143 | 41607 | 8:26647199 | 41617 | 8:26902337 |
| 41618 | 8:26902597 | 41619 | 8:26902692 | 41620 | 8:26906751 | 41621 | 8:26909158 | 41622 | 8:26909371 |
| 41663 | 8:28392311 | 41694 | 8:29260025 | 41703 | 8:29463858 | 41797 | 8:31800228 | 41798 | 8:31843921 |
| 41829 | 8:32784569 | 41840 | 8:33342641 | 41853 | 8:33651679 | 41868 | 8:34125341 | 41876 | 8:34286689 |
| 41883 | 8:34490228 | 41897 | 8:34815302 | 41907 | 8:35172469 | 41963 | 8:36234097 | 41982 | 8:36725916 |
| 41989 | 8:36958536 | 42015 | 8:37563030 | 42052 | 8:38794675 | 42072 | 8:39672661 | 42134 | 8:40784197 |
| 42138 | 8:40885929 | 42141 | 8:41023065 | 42185 | 8:42062463 | 42259 | 8:44042188 | 42291 | 8:45099245 |
| 42293 | 8:45139392 | 42318 | 8:45729304 | 42324 | 8:45797263 | 42335 | 8:46074143 | 42336 | 8:46102241 |
| 42337 | 8:46116001 | 42338 | 8:46116354 | 42339 | 8:46135520 | 42341 | 8:46137990 | 42342 | 8:46144578 |
| 42344 | 8:46153342 | 42345 | 8:46156573 | 42375 | 8:46982936 | 42420 | 8:48031781 | 42430 | 8:48479991 |
| 42539 | 8:51426634 | 42545 | 8:51576820 | 42575 | 8:52252692 | 42585 | 8:52524895 | 42602 | 8:52976427 |
| 42613 | 8:53298802 | 42616 | 8:53382084 | 42702 | 8:55675444 | 42724 | 8:56364736 | 42725 | 8:56366787 |
| 42751 | 8:56934422 | 42753 | 8:56941050 | 42754 | 8:57019107 | 42783 | 8:57597865 | 42810 | 8:58618980 |
| 42832 | 8:59315871 | 42871 | 8:60125622 | 42934 | 8:61718348 | 42938 | 8:61889487 | 42971 | 8:62566075 |
| 42972 | 8:62584738 | 42992 | 8:63183224 | 43052 | 8:64941395 | 43080 | 8:65506614 | 43107 | 8:66205907 |
| 43118 | 8:66471868 | 43138 | 8:66967439 | 43144 | 8:67016183 | 43176 | 8:67787815 | 43178 | 8:67793980 |
| 43188 | 8:67954514 | 43191 | 8:68086072 | 43206 | 8:68512363 | 43207 | 8:68517559 | 43214 | 8:68740750 |
| 43231 | 8:69106674 | 43234 | 8:69118901 | 43266 | 8:70045486 | 43271 | 8:70141611 | 43282 | 8:70418020 |
| 43313 | 8:71060104 | 43314 | 8:71090353 | 43325 | 8:71516540 | 43350 | 8:72260036 | 43357 | 8:72432728 |
| 43358 | 8:72437508 | 43365 | 8:72557656 | 43388 | 8:73136540 | 43419 | 8:73847683 | 43451 | 8:74692744 |
| 43498 | 8:76193941 | 43517 | 8:76534193 | 43528 | 8:76736188 | 43587 | 8:78346120 | 43592 | 8:78443361 |
| 43628 | 8:79886867 | 43672 | 8:81022175 | 43675 | 8:81050611 | 43723 | 8:82076199 | 43741 | 8:82402914 |
| 43744 | 8:82410990 | 43754 | 8:82684234 | 43775 | 8:83245271 | 43786 | 8:83673295 | 43800 | 8:83846337 |
| 43806 | 8:83935130 | 43889 | 8:86085819 | 43893 | 8:86142301 | 43906 | 8:86539317 | 43913 | 8:86746201 |
| 43924 | 8:86977892 | 43928 | 8:87127829 | 43929 | 8:87139627 | 43930 | 8:87141766 | 43931 | 8:87142536 |
| 43933 | 8:87168896 | 43934 | 8:87175091 | 43935 | 8:87177155 | 43936 | 8:87183548 | 43937 | 8:87200658 |
| 43938 | 8:87218682 | 43946 | 8:87439832 | 44026 | 8:89300462 | 44066 | 8:90333835 | 44083 | 8:90850582 |
| 44093 | 8:90959304 | 44126 | 8:91835974 | 44162 | 8:92493864 | 44184 | 8:93235330 | 44287 | 8:96565770 |
| 44307 | 8:97085820 | 44344 | 8:98025826 | 44346 | 8:98099120 | 44377 | 8:98925263 | 44383 | 8:99073097 |
| 44444 | 8:100321613 | 44447 | 8:100390505 | 44521 | 8:102187006 | 44544 | 8:102423408 | 44548 | 8:102462371 |
| 44563 | 8:102641248 | 44565 | 8:102643183 | 44575 | 8:102804781 | 44617 | 8:103596897 | 44665 | 8:104455217 |
| 44671 | 8:104552362 | 44698 | 8:105230189 | 44708 | 8:105413829 | 44750 | 8:106646055 | 44751 | 8:106656709 |
| 44775 | 8:107216243 | 44777 | 8:107231955 | 44791 | 8:107578002 | 44798 | 8:107830676 | 44819 | 8:108486026 |
| 44820 | 8:108568498 | 44825 | 8:108815568 | 44854 | 8:109956440 | 44867 | 8:110340060 | 44885 | 8:110872351 |
| 44889 | 8:110978542 | 44927 | 9:28156 | 44939 | 9:575925 | 44940 | 9:575959 | 44942 | 9:582062 |
| 44943 | 9:590791 | 44944 | 9:593091 | 44945 | 9:600069 | 44946 | 9:600393 | 44948 | 9:618129 |
| 44949 | 9:632185 | 44950 | 9:633412 | 44951 | 9:642243 | 44952 | 9:659431 | 44953 | 9:659947 |
| 44954 | 9:660978 | 44955 | 9:661495 | 44956 | 9:665906 | 44958 | 9:681361 | 44959 | 9:697171 |
| 44960 | 9:708137 | 44961 | 9:709297 | 44964 | 9:726333 | 44969 | 9:900985 | 45000 | 9:1570889 |
| 45010 | 9:1844977 | 45023 | 9:2164529 | 45053 | 9:3003204 | 45069 | 9:3571118 | 45103 | 9:4701519 |
| 45114 | 9:5085846 | 45144 | 9:5897942 | 45145 | 9:5911158 | 45148 | 9:6056118 | 45160 | 9:6416188 |
| 45193 | 9:7630506 | 45195 | 9:7644292 | 45210 | 9:7983021 | 45214 | 9:8111645 | 45256 | 9:9020374 |
| 45257 | 9:9029730 | 45403 | 9:11584044 | 45408 | 9:11752659 | 45448 | 9:12626683 | 45454 | 9:12671087 |
| 45455 | 9:12689678 | 45457 | 9:12695431 | 45458 | 9:12695934 | 45459 | 9:12695941 | 45460 | 9:12696018 |
| 45461 | 9:12696113 | 45462 | 9:12696251 | 45463 | 9:12696292 | 45464 | 9:12696440 | 45465 | 9:12696661 |
| 45466 | 9:12698815 | 45467 | 9:12698843 | 45468 | 9:12700184 | 45472 | 9:12770979 | 45479 | 9:13069374 |
| 45480 | 9:13082121 | 45482 | 9:13102812 | 45483 | 9:13103635 | 45484 | 9:13104067 | 45486 | 9:13108898 |
| 45487 | 9:13109246 | 45488 | 9:13109258 | 45489 | 9:13110088 | 45494 | 9:13242607 | 45533 | 9:14240906 |
| 45547 | 9:14661048 | 45581 | 9:15193969 | 45591 | 9:15445642 | 45614 | 9:15671238 | 45615 | 9:15672381 |
| 45616 | 9:15672435 | 45617 | 9:15672993 | 45618 | 9:15673156 | 45619 | 9:15673271 | 45620 | 9:15673402 |
| 45621 | 9:15674725 | 45632 | 9:15869515 | 45635 | 9:15935953 | 45639 | 9:15995486 | 45654 | 9:16430498 |
| 45663 | 9:16688073 | 45693 | 9:17598783 | 45694 | 9:17599118 | 45783 | 9:20470091 | 45787 | 9:20622642 |
| 45794 | 9:20764203 | 45820 | 9:21613566 | 45821 | 9:21636143 | 45822 | 9:21640871 | 45823 | 9:21643825 |
| 45825 | 9:21654717 | 45826 | 9:21683117 | 45827 | 9:21683679 | 45829 | 9:21715481 | 45830 | 9:21740090 |
| 45831 | 9:21757906 | 45855 | 9:22294337 | 45866 | 9:22593668 | 45869 | 9:22639958 | 45923 | 9:23824584 |
| 45927 | 9:23893279 | 45955 | 9:24560985 | 45994 | 9:25737610 | 45995 | 9:25738079 | 45996 | 9:25741377 |
| 46014 | 9:26124495 | 46025 | 9:26374532 | 46056 | 9:27294641 | 46078 | 9:27733478 | 46081 | 9:27779272 |
| 46083 | 9:27801667 | 46103 | 9:28362224 | 46104 | 9:28363030 | 46113 | 9:28567312 | 46114 | 9:28568804 |
| 46115 | 9:28569972 | 46116 | 9:28591418 | 46117 | 9:28593815 | 46122 | 9:28646308 | 46140 | 9:29091195 |
| 46143 | 9:29192292 | 46144 | 9:29204130 | 46145 | 9:29256950 | 46147 | 9:29283806 | 46148 | 9:29295585 |
| 46151 | 9:29380299 | 46152 | 9:29380683 | 46153 | 9:29381237 | 46154 | 9:29381255 | 46155 | 9:29383215 |
| 46156 | 9:29383228 | 46157 | 9:29383283 | 46158 | 9:29384303 | 46159 | 9:29384799 | 46160 | 9:29385786 |
| 46161 | 9:29385818 | 46162 | 9:29386059 | 46163 | 9:29386376 | 46164 | 9:29387098 | 46166 | 9:29400041 |
| 46167 | 9:29400182 | 46168 | 9:29402441 | 46169 | 9:29402650 | 46170 | 9:29403623 | 46171 | 9:29409271 |
| 46172 | 9:29411199 | 46187 | 9:29691912 | 46188 | 9:29692738 | 46199 | 9:30032893 | 46200 | 9:30033128 |
| 46218 | 9:30451686 | 46223 | 9:30612426 | 46225 | 9:30625188 | 46246 | 9:31013461 | 46311 | 9:32638638 |
| 46346 | 9:33419705 | 46367 | 9:33960698 | 46387 | 9:34556125 | 46410 | 9:35190440 | 46444 | 9:36105021 |
| 46494 | 9:37158301 | 46519 | 9:37870307 | 46552 | 9:38368263 | 46582 | 9:38867506 | 46583 | 9:38875675 |
| 46584 | 9:38878148 | 46586 | 9:38894269 | 46637 | 9:39995374 | 46695 | 9:41446116 | 46699 | 9:41532669 |
| 46739 | 9:42551675 | 46776 | 9:43626998 | 46784 | 9:43912263 | 46785 | 9:43915403 | 46798 | 9:44230197 |
| 46806 | 9:44371483 | 46812 | 9:44533430 | 46816 | 9:44595447 | 46859 | 9:46082473 | 46887 | 9:46922739 |
| 46902 | 9:47520187 | 46903 | 9:47553422 | 46907 | 9:47590125 | 46975 | 9:49674165 | 46989 | 9:50119411 |
| 47011 | 9:50615961 | 47022 | 9:50886609 | 47024 | 9:50944146 | 47064 | 9:52161063 | 47071 | 9:52377348 |
| 47106 | 9:53217250 | 47107 | 9:53217253 | 47108 | 9:53217815 | 47109 | 9:53217988 | 47110 | 9:53225739 |
| 47111 | 9:53226122 | 47112 | 9:53226145 | 47113 | 9:53227113 | 47114 | 9:53234782 | 47115 | 9:53245948 |
| 47116 | 9:53269708 | 47118 | 9:53277095 | 47119 | 9:53298978 | 47163 | 9:54707994 | 47167 | 9:54805840 |
| 47180 | 9:55233499 | 47206 | 9:56084986 | 47274 | 9:57805504 | 47290 | 9:58371472 | 47310 | 9:58992635 |
| 47323 | 9:59401110 | 47324 | 9:59402335 | 47327 | 9:59422003 | 47329 | 9:59459837 | 47373 | 9:60362161 |
| 47469 | 9:63010039 | 47474 | 9:63179604 | 47477 | 9:63266632 | 47524 | 9:64700448 | 47593 | 9:65489746 |
| 47601 | 9:65582850 | 47619 | 9:66094001 | 47639 | 9:66606853 | 47652 | 9:67044823 | 47659 | 9:67241727 |
| 47664 | 9:67366039 | 47677 | 9:67697561 | 47689 | 9:68036049 | 47693 | 9:68114580 | 47694 | 9:68115586 |
| 47713 | 9:68594580 | 47753 | 9:69635715 | 47757 | 9:69752727 | 47788 | 9:70415891 | 47795 | 9:70619172 |
| 47807 | 9:70987356 | 47808 | 9:70997161 | 47878 | 9:72597976 | 47889 | 9:72874182 | 47936 | 9:74030455 |
| 47949 | 9:74381423 | 47965 | 9:74748032 | 48044 | 9:76966345 | 48070 | 9:77485895 | 48073 | 9:77511368 |
| 48075 | 9:77523509 | 48078 | 9:77562662 | 48081 | 9:77694221 | 48083 | 9:77699388 | 48084 | 9:77713754 |
| 48085 | 9:77715782 | 48086 | 9:77716409 | 48087 | 9:77716687 | 48088 | 9:77717203 | 48089 | 9:77717466 |
| 48090 | 9:77718722 | 48092 | 9:77723623 | 48093 | 9:77728652 | 48094 | 9:77728956 | 48095 | 9:77729189 |
| 48096 | 9:77729397 | 48097 | 9:77729413 | 48098 | 9:77730079 | 48099 | 9:77730486 | 48100 | 9:77730739 |
| 48101 | 9:77730950 | 48102 | 9:77731339 | 48103 | 9:77731548 | 48104 | 9:77731587 | 48105 | 9:77731649 |
| 48106 | 9:77732824 | 48107 | 9:77733602 | 48108 | 9:77733985 | 48109 | 9:77734457 | 48110 | 9:77734537 |
| 48111 | 9:77734632 | 48112 | 9:77739942 | 48113 | 9:77740756 | 48114 | 9:77741688 | 48115 | 9:77742632 |
| 48116 | 9:77743840 | 48117 | 9:77744601 | 48118 | 9:77744620 | 48119 | 9:77745039 | 48120 | 9:77745584 |
| 48121 | 9:77745608 | 48122 | 9:77751329 | 48123 | 9:77751755 | 48124 | 9:77754808 | 48125 | 9:77756169 |
| 48126 | 9:77756446 | 48127 | 9:77756531 | 48128 | 9:77762645 | 48129 | 9:77764362 | 48132 | 9:77792668 |
| 48133 | 9:77804673 | 48134 | 9:77811111 | 48135 | 9:77811220 | 48136 | 9:77811579 | 48137 | 9:77811594 |
| 48138 | 9:77811612 | 48139 | 9:77811647 | 48140 | 9:77811795 | 48141 | 9:77812291 | 48142 | 9:77812385 |
| 48143 | 9:77812527 | 48144 | 9:77812542 | 48145 | 9:77812555 | 48146 | 9:77812585 | 48191 | 9:79533173 |
| 48196 | 9:79702375 | 48249 | 9:80821611 | 48264 | 9:81093450 | 48265 | 9:81096096 | 48282 | 9:81398472 |
| 48298 | 9:81743991 | 48302 | 9:81797320 | 48321 | 9:82185927 | 48326 | 9:82275767 | 48341 | 9:82639891 |
| 48346 | 9:82675266 | 48347 | 9:82681644 | 48349 | 9:82685499 | 48350 | 9:82696652 | 48351 | 9:82697299 |
| 48352 | 9:82699229 | 48353 | 9:82699333 | 48355 | 9:82706831 | 48356 | 9:82708646 | 48366 | 9:82981739 |
| 48375 | 9:83266367 | 48408 | 9:84082840 | 48409 | 9:84084042 | 48410 | 9:84084459 | 48411 | 9:84098275 |
| 48412 | 9:84100341 | 48413 | 9:84101062 | 48419 | 9:84280123 | 48430 | 9:84602199 | 48453 | 9:85206226 |
| 48527 | 9:86728832 | 48542 | 9:87041634 | 48554 | 9:87334131 | 48645 | 9:88942711 | 48697 | 9:89633025 |
| 48704 | 9:89813410 | 48724 | 9:90315206 | 48740 | 9:90776042 | 48765 | 9:91141962 | 48791 | 9:91739734 |
| 48805 | 9:92056243 | 48807 | 9:92137059 | 48974 | 9:95252173 | 48981 | 9:95442777 | 49012 | 9:96258094 |
| 49084 | 9:97052004 | 49086 | 9:97068291 | 49093 | 9:97195333 | 49115 | 9:97614153 | 49118 | 9:97651483 |
| 49158 | 9:98444344 | 49165 | 9:98565031 | 49183 | 9:99022247 | 49194 | 9:99214577 | 49227 | 9:99757934 |
| 49228 | 9:99766501 | 49286 | 9:100838600 | 49287 | 9:100865957 | 49301 | 9:101130910 | 49308 | 9:101233972 |
| 49316 | 9:101405391 | 49323 | 9:101487283 | 49399 | 9:102585986 | 49428 | 9:102905463 | 49443 | 9:103267046 |
| 49470 | 9:103818635 | 49557 | 10:1514567 | 49560 | 10:1608093 | 49571 | 10:1809005 | 49573 | 10:1812942 |
| 49575 | 10:1815628 | 49596 | 10:2181089 | 49600 | 10:2318770 | 49603 | 10:2355906 | 49633 | 10:2956009 |
| 49650 | 10:3286732 | 49685 | 10:3957621 | 49719 | 10:4637364 | 49756 | 10:5504897 | 49757 | 10:5506239 |
| 49758 | 10:5506479 | 49759 | 10:5506633 | 49760 | 10:5507158 | 49761 | 10:5507326 | 49762 | 10:5507349 |
| 49763 | 10:5507472 | 49764 | 10:5507500 | 49765 | 10:5507611 | 49766 | 10:5507931 | 49767 | 10:5507997 |
| 49768 | 10:5508125 | 49769 | 10:5508230 | 49770 | 10:5509205 | 49771 | 10:5509288 | 49772 | 10:5511487 |
| 49773 | 10:5511551 | 49774 | 10:5511726 | 49817 | 10:6459980 | 49835 | 10:6823353 | 49837 | 10:6940126 |
| 49899 | 10:8330854 | 49901 | 10:8385791 | 49917 | 10:8566513 | 49965 | 10:9790239 | 49972 | 10:9846509 |
| 49987 | 10:10261088 | 50046 | 10:11321931 | 50047 | 10:11322196 | 50076 | 10:12039647 | 50090 | 10:12326160 |
| 50091 | 10:12337486 | 50093 | 10:12394358 | 50099 | 10:12500777 | 50121 | 10:13059560 | 50124 | 10:13141850 |
| 50132 | 10:13207866 | 50133 | 10:13222364 | 50134 | 10:13224497 | 50135 | 10:13225359 | 50136 | 10:13226008 |
| 50139 | 10:13238081 | 50140 | 10:13248003 | 50142 | 10:13255208 | 50143 | 10:13258421 | 50144 | 10:13282620 |
| 50145 | 10:13286682 | 50149 | 10:13347534 | 50150 | 10:13347535 | 50179 | 10:13823541 | 50183 | 10:13888930 |
| 50189 | 10:13964255 | 50193 | 10:14079328 | 50212 | 10:14629981 | 50215 | 10:14653991 | 50257 | 10:15551922 |
| 50260 | 10:15625431 | 50298 | 10:16237750 | 50328 | 10:16751980 | 50350 | 10:17119224 | 50352 | 10:17147669 |
| 50367 | 10:17432966 | 50379 | 10:17732565 | 50396 | 10:18087252 | 50397 | 10:18089168 | 50398 | 10:18089791 |
| 50399 | 10:18089811 | 50400 | 10:18092432 | 50401 | 10:18095170 | 50402 | 10:18096798 | 50403 | 10:18100952 |
| 50404 | 10:18105123 | 50411 | 10:18246343 | 50458 | 10:19161271 | 50477 | 10:19610258 | 50508 | 10:20302691 |
| 50514 | 10:20365815 | 50579 | 10:21377001 | 50615 | 10:22113385 | 50620 | 10:22271591 | 50709 | 10:26380149 |
| 50714 | 10:26474079 | 50715 | 10:26474882 | 50741 | 10:26820943 | 50777 | 10:27459845 | 50848 | 10:28936817 |
| 50850 | 10:28939598 | 50861 | 10:29148690 | 50903 | 10:29990871 | 50973 | 10:32272335 | 50977 | 10:32347626 |
| 50980 | 10:32449180 | 50984 | 10:32556195 | 51013 | 10:33127535 | 51014 | 10:33187873 | 51029 | 10:33566653 |
| 51086 | 10:34900072 | 51089 | 10:34926072 | 51095 | 10:35028845 | 51152 | 10:36423193 | 51178 | 10:37020667 |
| 51199 | 10:37655975 | 51203 | 10:37686916 | 51241 | 10:38641559 | 51258 | 10:39024264 | 51342 | 10:41413651 |
| 51351 | 10:41730162 | 51352 | 10:41731424 | 51359 | 10:41898236 | 51375 | 10:42389264 | 51378 | 10:42430118 |
| 51380 | 10:42448787 | 51382 | 10:42452483 | 51383 | 10:42455131 | 51389 | 10:42580900 | 51404 | 10:43179077 |
| 51419 | 10:43825008 | 51479 | 10:45276327 | 51526 | 10:46079546 | 51575 | 10:47154407 | 51602 | 10:47651824 |
| 51624 | 10:48309146 | 51656 | 10:48956010 | 51683 | 10:49439054 | 51714 | 10:50089231 | 51803 | 10:52130146 |
| 51819 | 10:52557986 | 51853 | 10:53459983 | 51878 | 10:54196187 | 51893 | 10:54537058 | 51894 | 10:54542851 |
| 51931 | 10:55708254 | 51954 | 10:56299639 | 51978 | 10:56721716 | 51981 | 10:56843229 | 51982 | 10:56922663 |
| 51988 | 10:57043405 | 51991 | 10:57115514 | 52018 | 10:57976067 | 52081 | 10:59624803 | 52102 | 10:60442020 |
| 52108 | 10:60636369 | 52159 | 10:62050176 | 52174 | 10:62290589 | 52249 | 10:64233969 | 52267 | 10:64525088 |
| 52268 | 10:64536219 | 52269 | 10:64543768 | 52270 | 10:64548032 | 52271 | 10:64548906 | 52286 | 10:64979230 |
| 52295 | 10:65266598 | 52339 | 10:66097225 | 52340 | 10:66120437 | 52361 | 10:66674133 | 52362 | 10:66674580 |
| 52363 | 10:66675048 | 52364 | 10:66675130 | 52366 | 10:66770244 | 52422 | 10:68107692 | 52424 | 10:68140093 |
| 52444 | 10:68518650 | 52536 | 10:71021997 | 52557 | 10:71661492 | 52574 | 10:72103748 | 52600 | 10:73153480 |
| 52604 | 10:73217430 | 52615 | 10:73492848 | 52620 | 10:73620458 | 52681 | 10:75374747 | 52696 | 10:75774470 |
| 52699 | 10:75886187 | 52707 | 10:76141481 | 52708 | 10:76174432 | 52734 | 10:76609622 | 52735 | 10:76613357 |
| 52737 | 10:76718459 | 52746 | 10:76886184 | 52808 | 10:78222151 | 52816 | 10:78333529 | 52831 | 10:78834237 |
| 52838 | 10:78993174 | 52847 | 10:79256209 | 52855 | 10:79448706 | 52875 | 10:80033473 | 52893 | 10:80437796 |
| 52905 | 10:80755095 | 52952 | 10:81403359 | 53025 | 10:82946024 | 53076 | 10:84003158 | 53119 | 10:84929419 |
| 53136 | 10:85211692 | 53141 | 10:85308316 | 53146 | 10:85365819 | 53151 | 10:85506776 | 53153 | 10:85532563 |
| 53180 | 10:86322591 | 53190 | 10:86480002 | 53198 | 10:86564397 | 53234 | 10:87380593 | 53240 | 10:87570845 |
| 53303 | 10:88984928 | 53307 | 10:89076810 | 53328 | 10:89477438 | 53338 | 10:89716618 | 53376 | 10:90408131 |
| 53386 | 10:90606603 | 53387 | 10:90610286 | 53410 | 10:90869492 | 53412 | 10:90878413 | 53427 | 10:91270590 |
| 53500 | 10:92937814 | 53506 | 10:93026084 | 53510 | 10:93101284 | 53522 | 10:93436468 | 53527 | 10:93605955 |
| 53581 | 10:94985373 | 53592 | 10:95290678 | 53606 | 10:95624360 | 53629 | 10:96068605 | 53633 | 10:96193408 |
| 53649 | 10:96591095 | 53654 | 10:96786131 | 53655 | 10:96788386 | 53694 | 10:97467089 | 53707 | 10:97772610 |
| 53708 | 10:97775801 | 53709 | 10:97778298 | 53734 | 10:98439866 | 53759 | 10:98730218 | 53782 | 10:99444853 |
| 53785 | 10:99471490 | 53794 | 10:99596036 | 53811 | 10:99982401 | 53873 | 10:10113952 | 53898 | 10:10165041 |
| 53899 | 10:10166055 | 53900 | 10:10167602 | 53902 | 10:10170386 | 53916 | 10:10185170 | 53926 | 10:10206511 |
| 53928 | 10:10208682 | 53933 | 10:10213240 | 53939 | 10:10223950 | 53977 | 10:10315226 | 54020 | 11:779732 |
| 54030 | 11:923580 | 54035 | 11:1009994 | 54047 | 11:1211969 | 54118 | 11:2084360 | 54127 | 11:2256142 |
| 54133 | 11:2359945 | 54181 | 11:2799677 | 54196 | 11:3053638 | 54206 | 11:3153964 | 54238 | 11:3763768 |
| 54328 | 11:5718123 | 54345 | 11:6071587 | 54380 | 11:7143509 | 54398 | 11:7527861 | 54404 | 11:7599092 |
| 54441 | 11:8559109 | 54503 | 11:9819964 | 54512 | 11:10000902 | 54588 | 11:11731827 | 54594 | 11:11844298 |
| 54617 | 11:12454930 | 54653 | 11:13209044 | 54654 | 11:13222009 | 54689 | 11:13914152 | 54696 | 11:14200577 |
| 54727 | 11:15182386 | 54732 | 11:15285660 | 54749 | 11:15700826 | 54770 | 11:16056267 | 54804 | 11:16934584 |
| 54814 | 11:17117515 | 54867 | 11:18770128 | 54928 | 11:20656584 | 54943 | 11:21060821 | 54992 | 11:22018210 |
| 55000 | 11:22303794 | 55012 | 11:22569464 | 55020 | 11:22724476 | 55032 | 11:23083144 | 55068 | 11:23895849 |
| 55087 | 11:24338666 | 55097 | 11:24469097 | 55129 | 11:25273777 | 55140 | 11:25560609 | 55162 | 11:26154618 |
| 55165 | 11:26170393 | 55252 | 11:28239112 | 55304 | 11:29324448 | 55317 | 11:29530060 | 55324 | 11:29724736 |
| 55329 | 11:29759229 | 55355 | 11:30470011 | 55385 | 11:31068333 | 55434 | 11:32501555 | 55466 | 11:33302958 |
| 55503 | 11:34519584 | 55542 | 11:35824586 | 55552 | 11:36039319 | 55557 | 11:36151963 | 55562 | 11:36293996 |
| 55577 | 11:36723444 | 55599 | 11:37215399 | 55600 | 11:37240873 | 55626 | 11:37656828 | 55634 | 11:37841103 |
| 55637 | 11:37898906 | 55672 | 11:39016826 | 55701 | 11:40047215 | 55706 | 11:40222392 | 55716 | 11:40663028 |
| 55751 | 11:41311710 | 55752 | 11:41326914 | 55755 | 11:41328623 | 55758 | 11:41354523 | 55760 | 11:41356073 |
| 55766 | 11:41457760 | 55774 | 11:41843798 | 55796 | 11:42373804 | 55797 | 11:42374333 | 55798 | 11:42374619 |
| 55799 | 11:42374860 | 55800 | 11:42375434 | 55802 | 11:42378608 | 55803 | 11:42395653 | 55804 | 11:42395937 |
| 55808 | 11:42450574 | 55809 | 11:42458071 | 55826 | 11:42952869 | 55828 | 11:42977381 | 55834 | 11:43155397 |
| 55849 | 11:43447636 | 55855 | 11:43656266 | 55867 | 11:44164688 | 55871 | 11:44197049 | 55904 | 11:45053290 |
| 55950 | 11:46193646 | 55973 | 11:46703688 | 55976 | 11:46763745 | 55977 | 11:46763771 | 55978 | 11:46763772 |
| 55979 | 11:46763803 | 55980 | 11:46763967 | 55981 | 11:46764372 | 55982 | 11:46765309 | 55983 | 11:46769693 |
| 56013 | 11:47198478 | 56053 | 11:48210329 | 56122 | 11:48963797 | 56127 | 11:49061005 | 56128 | 11:49080138 |
| 56135 | 11:49179291 | 56138 | 11:49285957 | 56143 | 11:49404465 | 56147 | 11:49422373 | 56152 | 11:49575365 |
| 56153 | 11:49582523 | 56198 | 11:51041604 | 56201 | 11:51160828 | 56205 | 11:51255454 | 56206 | 11:51261576 |
| 56252 | 11:53012268 | 56253 | 11:53022205 | 56257 | 11:53214759 | 56272 | 11:53823211 | 56293 | 11:54411745 |
| 56301 | 11:54607804 | 56302 | 11:54611060 | 56303 | 11:54615185 | 56304 | 11:54619006 | 56305 | 11:54619299 |
| 56306 | 11:54621378 | 56307 | 11:54624272 | 56308 | 11:54625736 | 56309 | 11:54637534 | 56310 | 11:54640117 |
| 56311 | 11:54640249 | 56312 | 11:54644177 | 56313 | 11:54658429 | 56314 | 11:54667025 | 56315 | 11:54672210 |
| 56317 | 11:54687119 | 56318 | 11:54696136 | 56327 | 11:54925623 | 56341 | 11:55201147 | 56345 | 11:55322961 |
| 56384 | 11:56468221 | 56394 | 11:56809772 | 56395 | 11:56811610 | 56396 | 11:56812388 | 56408 | 11:57181716 |
| 56434 | 11:58004110 | 56441 | 11:58172817 | 56451 | 11:58552292 | 56452 | 11:58557598 | 56478 | 11:59155112 |
| 56498 | 11:59762890 | 56519 | 11:60453153 | 56528 | 11:60471884 | 56539 | 11:60491356 | 56609 | 11:62266387 |
| 56611 | 11:62305455 | 56612 | 11:62308757 | 56614 | 11:62379499 | 56617 | 11:62426969 | 56623 | 11:62609754 |
| 56681 | 11:63826838 | 56692 | 11:63976694 | 56707 | 11:64337696 | 56711 | 11:64464647 | 56737 | 11:65172790 |
| 56789 | 11:66422364 | 56887 | 11:68143046 | 56912 | 11:68896819 | 56918 | 11:69065684 | 56919 | 11:69065947 |
| 56920 | 11:69067967 | 56927 | 11:69141312 | 56934 | 11:69198908 | 56936 | 11:69213888 | 56965 | 11:69725037 |
| 56975 | 11:69945179 | 56979 | 11:70105382 | 56989 | 11:70270717 | 56991 | 11:70291428 | 57061 | 11:71167695 |
| 57063 | 11:71169475 | 57064 | 11:71171201 | 57065 | 11:71174044 | 57071 | 11:71187848 | 57083 | 11:71410081 |
| 57092 | 11:71596359 | 57093 | 11:71603815 | 57097 | 11:71735550 | 57118 | 11:72456335 | 57127 | 11:72799464 |
| 57171 | 11:73648343 | 57267 | 11:75692626 | 57288 | 11:76333610 | 57314 | 11:77013992 | 57318 | 11:77067925 |
| 57319 | 11:77077199 | 57320 | 11:77085172 | 57330 | 11:77232878 | 57340 | 11:77445226 | 57341 | 11:77485470 |
| 57396 | 11:78194981 | 57405 | 11:78326150 | 57555 | 11:81830178 | 57571 | 11:82150919 | 57595 | 11:82670314 |
| 57617 | 11:82895182 | 57620 | 11:82943647 | 57646 | 11:83482447 | 57648 | 11:83552676 | 57708 | 11:85258209 |
| 57718 | 11:85436750 | 57725 | 11:85653522 | 57730 | 11:85743559 | 57754 | 11:86206294 | 57755 | 11:86214871 |
| 57814 | 11:87707465 | 57847 | 11:87999059 | 57865 | 11:88032183 | 57884 | 11:88448012 | 57927 | 11:89546657 |
| 57946 | 11:89996070 | 57970 | 11:90503652 | 58023 | 11:91710069 | 58027 | 11:91791559 | 58028 | 11:91792786 |
| 58029 | 11:91796326 | 58030 | 11:91798254 | 58031 | 11:91807646 | 58032 | 11:91809950 | 58033 | 11:91809976 |
| 58042 | 11:92019924 | 58082 | 11:92823026 | 58091 | 11:93153121 | 58092 | 11:93165769 | 58093 | 11:93179602 |
| 58094 | 11:93185994 | 58100 | 11:93232642 | 58105 | 11:93261434 | 58106 | 11:93262306 | 58107 | 11:93262884 |
| 58108 | 11:93263825 | 58109 | 11:93279097 | 58111 | 11:93395136 | 58124 | 11:93858711 | 58143 | 11:94337656 |
| 58163 | 11:94860855 | 58168 | 11:95063641 | 58175 | 11:95296518 | 58268 | 11:97728887 | 58299 | 11:98222359 |
| 58303 | 11:98359510 | 58373 | 11:99755436 | 58492 | 11:10201461 | 58517 | 11:10257709 | 58524 | 11:10265965 |
| 58583 | 11:10338389 | 58596 | 11:10369741 | 58630 | 11:10447397 | 58644 | 11:10468322 | 58654 | 11:10482995 |
| 58656 | 11:10487555 | 58658 | 11:10492751 | 58661 | 11:10497336 | 58663 | 11:10497439 | 58681 | 11:10548246 |
| 58729 | 11:10679315 | 58731 | 11:10694859 | 58752 | 12:625880 | 58780 | 12:1498811 | 58821 | 12:2369495 |
| 58870 | 12:3405573 | 58875 | 12:3491002 | 58894 | 12:4026056 | 58925 | 12:4736478 | 58934 | 12:5016508 |
| 58960 | 12:5810001 | 59000 | 12:7239138 | 59032 | 12:8559085 | 59098 | 12:10166200 | 59115 | 12:10466559 |
| 59148 | 12:11055011 | 59172 | 12:11660529 | 59177 | 12:11743244 | 59237 | 12:13369400 | 59275 | 12:14073643 |
| 59284 | 12:14200873 | 59334 | 12:15275995 | 59404 | 12:16588996 | 59405 | 12:16617595 | 59445 | 12:17717003 |
| 59511 | 12:19127423 | 59512 | 12:19133184 | 59553 | 12:20149260 | 59563 | 12:20389759 | 59565 | 12:20491988 |
| 59566 | 12:20500610 | 59608 | 12:21574455 | 59700 | 12:23970417 | 59718 | 12:24380235 | 59730 | 12:24612900 |
| 59753 | 12:25107687 | 59760 | 12:25333357 | 59778 | 12:25727678 | 59779 | 12:25742060 | 59782 | 12:25755570 |
| 59787 | 12:25862626 | 59816 | 12:26904714 | 59834 | 12:27216744 | 59891 | 12:28914720 | 59952 | 12:30585785 |
| 59960 | 12:30737586 | 59964 | 12:30851151 | 59965 | 12:30851590 | 59978 | 12:31055012 | 60064 | 12:33121234 |
| 60068 | 12:33212754 | 60115 | 12:34706439 | 60158 | 12:35595816 | 60205 | 12:37118156 | 60252 | 12:39318432 |
| 60253 | 12:39337804 | 60254 | 12:39339971 | 60255 | 12:39375252 | 60295 | 12:40436881 | 60299 | 12:40699170 |
| 60310 | 12:41000477 | 60345 | 12:42446263 | 60346 | 12:42529266 | 60384 | 12:44349627 | 60414 | 12:45309570 |
| 60464 | 12:46902819 | 60503 | 12:47894827 | 60512 | 12:48234155 | 60524 | 12:48552141 | 60528 | 12:48621944 |
| 60557 | 12:49347591 | 60588 | 12:50012484 | 60654 | 12:51413041 | 60677 | 12:52057233 | 60712 | 12:52833730 |
| 60713 | 12:52841272 | 60714 | 12:52843772 | 60715 | 12:52859350 | 60717 | 12:52867407 | 60718 | 12:52868520 |
| 60720 | 12:52883259 | 60721 | 12:52888503 | 60736 | 12:53324225 | 60737 | 12:53326935 | 60748 | 12:53513237 |
| 60776 | 12:54070308 | 60778 | 12:54112016 | 60800 | 12:54537212 | 60823 | 12:55077299 | 60834 | 12:55543664 |
| 60864 | 12:56465958 | 60881 | 12:57046134 | 60949 | 12:59148039 | 60950 | 12:59163748 | 60958 | 12:59442298 |
| 61087 | 12:63768535 | 61092 | 12:63878306 | 61108 | 12:64352510 | 61118 | 12:64738377 | 61196 | 12:66786596 |
| 61240 | 12:67928955 | 61247 | 12:68098257 | 61248 | 12:68114447 | 61259 | 12:68270144 | 61275 | 12:68608809 |
| 61278 | 12:68710557 | 61290 | 12:68863135 | 61301 | 12:69019005 | 61309 | 12:69286353 | 61311 | 12:69340936 |
| 61318 | 12:69467691 | 61328 | 12:69553759 | 61332 | 12:69582989 | 61366 | 12:71235763 | 61382 | 12:71600338 |
| 61412 | 12:72958303 | 61413 | 12:72961991 | 61446 | 12:73518731 | 61482 | 12:74197569 | 61497 | 12:74496338 |
| 61522 | 12:75101487 | 61529 | 12:75242221 | 61532 | 12:75277582 | 61537 | 12:75375680 | 61590 | 12:76682892 |
| 61618 | 12:77393088 | 61638 | 12:77847200 | 61645 | 12:77993107 | 61673 | 12:78662420 | 61704 | 12:79284630 |
| 61718 | 12:79542780 | 61728 | 12:79738869 | 61749 | 12:80221064 | 61750 | 12:80223568 | 61751 | 12:80224696 |
| 61755 | 12:80311844 | 61760 | 12:80405755 | 61762 | 12:80523810 | 61818 | 12:81778398 | 61836 | 12:82156928 |
| 61850 | 12:82530033 | 61865 | 12:82796759 | 61888 | 12:83319494 | 61922 | 12:84042281 | 61956 | 12:84892891 |
| 61968 | 12:85033607 | 61980 | 12:85234076 | 61998 | 12:85446719 | 62009 | 12:85641586 | 62011 | 12:85752928 |
| 62060 | 12:86779904 | 62103 | 13:648927 | 62135 | 13:1384950 | 62142 | 13:1583266 | 62183 | 13:2254372 |
| 62213 | 13:2708064 | 62260 | 13:3861084 | 62336 | 13:5664259 | 62337 | 13:5666787 | 62361 | 13:6275220 |
| 62366 | 13:6361450 | 62408 | 13:7483031 | 62413 | 13:7556779 | 62434 | 13:8333741 | 62438 | 13:8415584 |
| 62443 | 13:8529126 | 62444 | 13:8533157 | 62445 | 13:8537080 | 62446 | 13:8542772 | 62450 | 13:8682255 |
| 62451 | 13:8682878 | 62452 | 13:8683708 | 62453 | 13:8685414 | 62454 | 13:8686278 | 62524 | 13:10400290 |
| 62526 | 13:10426820 | 62537 | 13:10575199 | 62553 | 13:11047542 | 62573 | 13:12140250 | 62613 | 13:13240348 |
| 62654 | 13:14380630 | 62665 | 13:14649538 | 62669 | 13:14768979 | 62675 | 13:14878218 | 62690 | 13:15298554 |
| 62693 | 13:15509230 | 62707 | 13:15907128 | 62845 | 13:18649928 | 62849 | 13:18704268 | 62880 | 13:18741567 |
| 62886 | 13:18795212 | 62893 | 13:18926634 | 62904 | 13:19113938 | 62927 | 13:19611692 | 62953 | 13:19991782 |
| 62955 | 13:20085636 | 62962 | 13:20299025 | 62985 | 13:20633696 | 62991 | 13:20728403 | 62999 | 13:21031730 |
| 63014 | 13:21160488 | 63016 | 13:21199075 | 63039 | 13:21675803 | 63072 | 13:22491633 | 63080 | 13:22645333 |
| 63142 | 13:24652712 | 63154 | 13:24964331 | 63170 | 13:25313389 | 63233 | 13:26864651 | 63296 | 13:28352469 |
| 63310 | 13:28677951 | 63311 | 13:28716131 | 63313 | 13:28719397 | 63314 | 13:28721486 | 63315 | 13:28726672 |
| 63328 | 13:29164280 | 63331 | 13:29203677 | 63409 | 13:30734642 | 63413 | 13:30835419 | 63417 | 13:30928296 |
| 63433 | 13:31170980 | 63479 | 13:32132576 | 63480 | 13:32133958 | 63481 | 13:32143886 | 63482 | 13:32152278 |
| 63494 | 13:32274709 | 63506 | 13:32489566 | 63521 | 13:32651702 | 63522 | 13:32653621 | 63523 | 13:32654103 |
| 63528 | 13:32846791 | 63529 | 13:32846825 | 63530 | 13:32847172 | 63531 | 13:32847400 | 63542 | 13:33084958 |
| 63543 | 13:33086348 | 63572 | 13:33880293 | 63599 | 13:34800113 | 63619 | 13:35179138 | 63622 | 13:35197458 |
| 63766 | 13:38636130 | 63767 | 13:38662619 | 63768 | 13:38676202 | 63769 | 13:38680784 | 63775 | 13:38810147 |
| 63781 | 13:38839160 | 63782 | 13:38893419 | 63786 | 13:38949069 | 63796 | 13:39090329 | 63800 | 13:39121053 |
| 63832 | 13:39696126 | 63852 | 13:40100334 | 63860 | 13:40220102 | 63870 | 13:40453192 | 63872 | 13:40473511 |
| 63873 | 13:40486594 | 63874 | 13:40486661 | 63940 | 13:42071486 | 63972 | 13:42587452 | 63982 | 13:42606861 |
| 64003 | 13:42825481 | 64024 | 13:43516130 | 64032 | 13:43689434 | 64033 | 13:43726105 | 64039 | 13:43774352 |
| 64059 | 13:44057830 | 64060 | 13:44115215 | 64105 | 13:44933952 | 64122 | 13:44991547 | 64157 | 13:45403608 |
| 64176 | 13:45936548 | 64210 | 13:46658312 | 64216 | 13:46758989 | 64245 | 13:47366925 | 64250 | 13:47472066 |
| 64254 | 13:47495500 | 64264 | 13:47827146 | 64284 | 13:48238821 | 64285 | 13:48240137 | 64286 | 13:48240290 |
| 54287 | 13:48240322 | 64288 | 13:48240351 | 64289 | 13:48241221 | 64290 | 13:48241373 | 64291 | 13:48241390 |
| 64292 | 13:48243603 | 64293 | 13:48245013 | 64294 | 13:48245053 | 64296 | 13:48269729 | 64297 | 13:48274446 |
| 64318 | 13:49199089 | 64319 | 13:49199105 | 64320 | 13:49200231 | 64419 | 13:52063310 | 64429 | 13:52466434 |
| 64496 | 13:53368735 | 64499 | 13:53503066 | 64516 | 13:54246719 | 64522 | 13:54512037 | 64533 | 13:54732415 |
| 64593 | 13:56026065 | 64649 | 13:57529999 | 64650 | 13:57541123 | 64669 | 13:58019158 | 64698 | 13:58633062 |
| 64708 | 13:58899200 | 64721 | 13:59016374 | 64732 | 13:59248087 | 64735 | 13:59289762 | 64766 | 13:60057972 |
| 64776 | 13:60212933 | 64798 | 13:60664451 | 64807 | 13:60980294 | 64822 | 13:61522788 | 64846 | 13:62118304 |
| 64852 | 13:62277677 | 64888 | 13:62739693 | 64889 | 13:62752247 | 64893 | 13:62800301 | 64899 | 13:62843052 |
| 64946 | 13:63972761 | 64967 | 13:64523897 | 64988 | 13:65061778 | 65005 | 13:65480528 | 65011 | 13:65604335 |
| 65014 | 13:65656406 | 65021 | 13:65714427 | 65023 | 13:65715682 | 65033 | 13:65806803 | 65052 | 13:66249739 |
| 65070 | 13:66636049 | 65076 | 13:66754479 | 65077 | 13:66811790 | 65082 | 13:66930495 | 65099 | 13:67376538 |
| 65112 | 13:67740112 | 65114 | 13:67768319 | 65118 | 13:67910634 | 65141 | 13:68545291 | 65177 | 13:69533712 |
| 65193 | 13:69946188 | 65219 | 13:70559101 | 65254 | 13:71281225 | 65255 | 13:71297993 | 65264 | 13:71463110 |
| 65285 | 13:71937940 | 65291 | 13:72156689 | 65345 | 13:73285435 | 65394 | 13:74233601 | 65400 | 13:74368669 |
| 65402 | 13:74389287 | 65435 | 13:74875799 | 65458 | 13:75539335 | 65459 | 13:75546891 | 65460 | 13:75550544 |
| 65475 | 13:75773530 | 65490 | 13:76048819 | 65495 | 13:76206814 | 65497 | 13:76256932 | 65513 | 13:76540633 |
| 65533 | 13:76870141 | 65610 | 13:78594569 | 65632 | 13:78943793 | 65642 | 13:79095868 | 65658 | 13:79307341 |
| 65667 | 13:79411595 | 65668 | 13:79427565 | 65687 | 13:79896093 | 65693 | 13:80009218 | 65699 | 13:80110062 |
| 65700 | 13:80110693 | 65701 | 13:80112377 | 65702 | 13:80112537 | 65703 | 13:80112873 | 65704 | 13:80113035 |
| 65705 | 13:80113195 | 65706 | 13:80119481 | 65707 | 13:80120615 | 65708 | 13:80121232 | 65709 | 13:80121378 |
| 65710 | 13:80121492 | 65711 | 13:80121527 | 65712 | 13:80122117 | 65713 | 13:80123053 | 65714 | 13:80123496 |
| 65715 | 13:80123540 | 65716 | 13:80128525 | 65717 | 13:80128577 | 65718 | 13:80128592 | 65719 | 13:80128724 |
| 65720 | 13:80133414 | 65726 | 13:80284611 | 65730 | 13:80353010 | 65731 | 13:80354577 | 65741 | 13:80564883 |
| 65792 | 13:81650045 | 65813 | 13:82057542 | 65814 | 13:82060563 | 65847 | 13:82692047 | 65937 | 14:1748354 |
| 65939 | 14:1780705 | 65960 | 14:2169492 | 65962 | 14:2196270 | 65979 | 14:2821082 | 65999 | 14:3180090 |
| 66000 | 14:3180092 | 66001 | 14:3184037 | 66003 | 14:3188747 | 66031 | 14:3341005 | 66057 | 14:3777934 |
| 66059 | 14:3807311 | 66065 | 14:3937653 | 66096 | 14:4438274 | 66107 | 14:4826114 | 66109 | 14:4845905 |
| 66112 | 14:4900819 | 66114 | 14:4924180 | 66123 | 14:5079999 | 66141 | 14:5424566 | 66143 | 14:5442736 |
| 66144 | 14:5442915 | 66155 | 14:5642922 | 66161 | 14:5745989 | 66162 | 14:5746008 | 66163 | 14:5746079 |
| 66164 | 14:5746088 | 66167 | 14:5767279 | 66168 | 14:5767316 | 66182 | 14:6051710 | 66222 | 14:6580835 |
| 66251 | 14:7127015 | 66255 | 14:7190153 | 66260 | 14:7315224 | 66267 | 14:7446316 | 66296 | 14:7973769 |
| 66298 | 14:7981735 | 66299 | 14:7984017 | 66301 | 14:7993501 | 66303 | 14:8000324 | 66306 | 14:8006167 |
| 66307 | 14:8008471 | 66309 | 14:8029440 | 66324 | 14:8315882 | 66339 | 14:8555196 | 66342 | 14:8578594 |
| 66401 | 14:9370788 | 66424 | 14:9883880 | 66445 | 14:10330270 | 66487 | 14:11125973 | 66519 | 14:11675980 |
| 66520 | 14:11687647 | 66521 | 14:11700111 | 66536 | 14:12017220 | 66572 | 14:12683380 | 66573 | 14:12683869 |
| 66575 | 14:12685152 | 66576 | 14:12685153 | 66580 | 14:12718898 | 66581 | 14:12726976 | 66582 | 14:12726981 |
| 66642 | 14:14433818 | 66674 | 14:15281409 | 66683 | 14:15476637 | 66707 | 14:15871072 | 66740 | 14:16464011 |
| 66800 | 14:17774684 | 66848 | 14:18880176 | 66911 | 14:20615962 | 66917 | 14:20673221 | 66920 | 14:20723405 |
| 66921 | 14:20728338 | 66939 | 14:21119658 | 66940 | 14:21124340 | 66973 | 14:21853648 | 67075 | 14:22718406 |
| 67158 | 14:23199725 | 67159 | 14:23200416 | 67160 | 14:23200440 | 67161 | 14:23202111 | 67162 | 14:23202373 |
| 67163 | 14:23202574 | 67164 | 14:23202642 | 67165 | 14:23202665 | 67166 | 14:23205217 | 67167 | 14:23206165 |
| 67168 | 14:23209278 | 67169 | 14:23210217 | 67170 | 14:23211671 | 67171 | 14:23212276 | 67172 | 14:23212424 |
| 67174 | 14:23216603 | 67176 | 14:23216967 | 67177 | 14:23217298 | 67178 | 14:23217676 | 67179 | 14:23217682 |
| 67182 | 14:23217984 | 67184 | 14:23219378 | 67185 | 14:23220306 | 67186 | 14:23221060 | 67187 | 14:23221105 |
| 67188 | 14:23221660 | 67190 | 14:23223448 | 67192 | 14:23232920 | 67293 | 14:23296927 | 67353 | 14:23590382 |
| 67532 | 14:25538462 | 67551 | 14:25555299 | 67553 | 14:25557838 | 67562 | 14:25875465 | 67572 | 14:26025356 |
| 67592 | 14:26183095 | 67615 | 14:26313111 | 67631 | 14:26716130 | 67638 | 14:26937569 | 67647 | 14:27145363 |
| 67659 | 14:27578987 | 67666 | 14:27804049 | 67669 | 14:27925661 | 67680 | 14:28283881 | 67684 | 14:28475888 |
| 67701 | 14:28805553 | 67713 | 14:29204640 | 67728 | 14:29506987 | 67737 | 14:29651348 | 67751 | 14:30011410 |
| 67787 | 14:30531165 | 67806 | 14:31151661 | 67810 | 14:31229071 | 67812 | 14:31276668 | 67813 | 14:31348707 |
| 67839 | 14:32042320 | 67840 | 14:32080892 | 67847 | 14:32295836 | 67848 | 14:32298088 | 67849 | 14:32298121 |
| 67850 | 14:32298404 | 67851 | 14:32298532 | 67852 | 14:32298550 | 67853 | 14:32298592 | 67854 | 14:32298623 |
| 67855 | 14:32298715 | 67856 | 14:32298807 | 67857 | 14:32298889 | 67858 | 14:32298929 | 67859 | 14:32298949 |
| 67860 | 14:32298983 | 67861 | 14:32301242 | 67862 | 14:32301629 | 67863 | 14:32301804 | 67880 | 14:32768091 |
| 67902 | 14:33283287 | 67914 | 14:33481134 | 67945 | 14:34107991 | 67963 | 14:34616797 | 67966 | 14:34703575 |
| 67968 | 14:34726901 | 68040 | 14:36456652 | 68066 | 14:37335063 | 68131 | 14:39204823 | 68180 | 14:40564678 |
| 68185 | 14:40761575 | 68199 | 14:41245865 | 68207 | 14:41477789 | 68211 | 14:41608564 | 68246 | 14:42371257 |
| 68252 | 14:42499551 | 68275 | 14:43217965 | 68314 | 14:44597633 | 68367 | 14:45598918 | 68386 | 14:45953548 |
| 68393 | 14:46043440 | 68412 | 14:46302755 | 68430 | 14:46746014 | 68435 | 14:46846924 | 68463 | 14:47562082 |
| 68479 | 14:48154116 | 68495 | 14:48422440 | 68496 | 14:48424765 | 68507 | 14:48722429 | 68578 | 14:50964252 |
| 68611 | 14:51741282 | 68659 | 14:53067901 | 68716 | 14:54352030 | 68717 | 14:54359397 | 68727 | 14:54470262 |
| 68733 | 14:54629600 | 68785 | 14:56030241 | 68869 | 14:58169542 | 68876 | 14:58268988 | 68879 | 14:58347367 |
| 68898 | 14:58795728 | 68902 | 14:58882362 | 68914 | 14:59072940 | 68931 | 14:59422421 | 68945 | 14:59890123 |
| 68960 | 14:60186718 | 68998 | 14:61242862 | 69030 | 14:61805601 | 69069 | 14:62451453 | 69075 | 14:62584372 |
| 69079 | 14:62700380 | 69092 | 14:62957699 | 69106 | 14:63240134 | 69117 | 14:63473862 | 69160 | 14:64610567 |
| 69174 | 14:65146328 | 69215 | 14:66160998 | 69219 | 14:66259689 | 69228 | 14:66384994 | 69232 | 14:66419660 |
| 69326 | 14:68515393 | 69354 | 14:69351733 | 69370 | 14:69913790 | 69382 | 14:70082152 | 69391 | 14:70201845 |
| 69400 | 14:70384514 | 69408 | 14:70595169 | 69415 | 14:70881552 | 69416 | 14:70890232 | 69427 | 14:71123353 |
| 69443 | 14:71687136 | 69472 | 14:72410713 | 69494 | 14:72897923 | 69506 | 14:73212493 | 69515 | 14:73427877 |
| 69517 | 14:73468390 | 69534 | 14:73985016 | 69570 | 14:75029436 | 69571 | 14:75034802 | 69572 | 14:75035607 |
| 69573 | 14:75035827 | 69574 | 14:75035857 | 69575 | 14:75035923 | 69608 | 14:76029716 | 69631 | 14:76258289 |
| 69633 | 14:76268094 | 69680 | 14:77464768 | 69686 | 14:77542458 | 69690 | 14:77708662 | 69700 | 14:77954720 |
| 69736 | 14:78637209 | 69737 | 14:78644200 | 69742 | 14:78699024 | 69750 | 14:78929919 | 69762 | 14:79335372 |
| 69767 | 14:79486373 | 69783 | 14:79820967 | 69790 | 14:79974645 | 69805 | 14:80236030 | 69819 | 14:80591048 |
| 69864 | 14:81465207 | 69901 | 14:82003511 | 70000 | 15:2555400 | 70037 | 15:3651481 | 70043 | 15:3796804 |
| 70044 | 15:3859257 | 70045 | 15:3861053 | 70046 | 15:3861177 | 70048 | 15:3872949 | 70049 | 15:3874148 |
| 70050 | 15:3874413 | 70051 | 15:3875123 | 70052 | 15:3875291 | 70053 | 15:3875480 | 70054 | 15:3875800 |
| 70055 | 15:3876161 | 70056 | 15:3876211 | 70057 | 15:3876259 | 70058 | 15:3884992 | 70059 | 15:3885455 |
| 70060 | 15:3888242 | 70061 | 15:3888270 | 70070 | 15:4006347 | 70080 | 15:4274305 | 70107 | 15:4817619 |
| 70108 | 15:4821833 | 70109 | 15:4827554 | 70110 | 15:4850527 | 70111 | 15:4855973 | 70113 | 15:4859691 |
| 70114 | 15:4874530 | 70132 | 15:5345428 | 70133 | 15:5345893 | 70134 | 15:5346513 | 70135 | 15:5346544 |
| 70136 | 15:5347645 | 70137 | 15:5348421 | 70138 | 15:5348505 | 70139 | 15:5348800 | 70140 | 15:5348852 |
| 70141 | 15:5348875 | 70142 | 15:5349143 | 70143 | 15:5349849 | 70144 | 15:5350256 | 70145 | 15:5350323 |
| 70146 | 15:5350355 | 70147 | 15:5353059 | 70148 | 15:5353099 | 70149 | 15:5355811 | 70150 | 15:5355909 |
| 70151 | 15:5356157 | 70152 | 15:5356186 | 70153 | 15:5356365 | 70154 | 15:5356372 | 70155 | 15:5357215 |
| 70156 | 15:5358118 | 70157 | 15:5358230 | 70158 | 15:5358668 | 70159 | 15:5359593 | 70160 | 15:5359865 |
| 70161 | 15:5359868 | 70162 | 15:5361660 | 70163 | 15:5361661 | 70164 | 15:5365576 | 70165 | 15:5380375 |
| 70166 | 15:5381250 | 70167 | 15:5382096 | 70168 | 15:5383165 | 70169 | 15:5383541 | 70170 | 15:5384183 |
| 70171 | 15:5384848 | 70172 | 15:5384963 | 70173 | 15:5384997 | 70174 | 15:5385062 | 70175 | 15:5385270 |
| 70176 | 15:5385290 | 70177 | 15:5385312 | 70178 | 15:5385429 | 70179 | 15:5386627 | 70180 | 15:5386633 |
| 70181 | 15:5386655 | 70182 | 15:5386721 | 70183 | 15:5387773 | 70184 | 15:5387850 | 70185 | 15:5388162 |
| 70186 | 15:5388165 | 70187 | 15:5388635 | 70188 | 15:5388661 | 70189 | 15:5388748 | 70190 | 15:5389424 |
| 70191 | 15:5389588 | 70192 | 15:5389616 | 70193 | 15:5389859 | 70194 | 15:5389978 | 70195 | 15:5390296 |
| 70196 | 15:5390320 | 70197 | 15:5390450 | 70198 | 15:5390553 | 70199 | 15:5390863 | 70200 | 15:5390921 |
| 70201 | 15:5391192 | 70202 | 15:5391298 | 70203 | 15:5391508 | 70204 | 15:5391682 | 70205 | 15:5391799 |
| 70206 | 15:5392452 | 70207 | 15:5392859 | 70208 | 15:5392923 | 70209 | 15:5393194 | 70210 | 15:5393401 |
| 70211 | 15:5393671 | 70212 | 15:5393753 | 70213 | 15:5394283 | 70214 | 15:5395547 | 70215 | 15:5395610 |
| 70216 | 15:5395743 | 70217 | 15:5395751 | 70218 | 15:5395855 | 70219 | 15:5395947 | 70220 | 15:5398109 |
| 70221 | 15:5398341 | 70222 | 15:5398346 | 70223 | 15:5398513 | 70224 | 15:5398559 | 70225 | 15:5400626 |
| 70226 | 15:5401120 | 70227 | 15:5401525 | 70228 | 15:5401536 | 70229 | 15:5401568 | 70230 | 15:5401820 |
| 70231 | 15:5401853 | 70232 | 15:5404746 | 70233 | 15:5404825 | 70234 | 15:5405498 | 70235 | 15:5405505 |
| 70236 | 15:5406067 | 70237 | 15:5406105 | 70238 | 15:5406213 | 70239 | 15:5406470 | 70240 | 15:5406961 |
| 70241 | 15:5407075 | 70242 | 15:5407142 | 70243 | 15:5407235 | 70244 | 15:5407350 | 70245 | 15:5407539 |
| 70246 | 15:5407760 | 70247 | 15:5407841 | 70248 | 15:5407895 | 70249 | 15:5408434 | 70250 | 15:5408552 |
| 70251 | 15:5408640 | 70252 | 15:5408900 | 70253 | 15:5409527 | 70254 | 15:5409532 | 70255 | 15:5409637 |
| 70256 | 15:5409728 | 70257 | 15:5409914 | 70258 | 15:5410082 | 70259 | 15:5410166 | 70260 | 15:5410173 |
| 70261 | 15:5410337 | 70262 | 15:5410342 | 70263 | 15:5410429 | 70264 | 15:5410813 | 70265 | 15:5410848 |
| 70266 | 15:5412771 | 70267 | 15:5412796 | 70268 | 15:5412835 | 70269 | 15:5412869 | 70270 | 15:5413090 |
| 70271 | 15:5413111 | 70272 | 15:5413650 | 70273 | 15:5415034 | 70274 | 15:5415477 | 70275 | 15:5416298 |
| 70276 | 15:5416324 | 70277 | 15:5417129 | 70278 | 15:5417450 | 70279 | 15:5417663 | 70280 | 15:5418542 |
| 70284 | 15:5503948 | 70303 | 15:6004286 | 70319 | 15:6435496 | 70337 | 15:7003211 | 70338 | 15:7008452 |
| 70340 | 15:7019231 | 70341 | 15:7029657 | 70342 | 15:7033643 | 70348 | 15:7265163 | 70349 | 15:7265165 |
| 70350 | 15:7265341 | 70351 | 15:7265425 | 70352 | 15:7265838 | 70353 | 15:7270627 | 70354 | 15:7270650 |
| 70355 | 15:7272155 | 70356 | 15:7272347 | 70357 | 15:7272651 | 70358 | 15:7275537 | 70359 | 15:7275789 |
| 70360 | 15:7275947 | 70361 | 15:7276254 | 70362 | 15:7277640 | 70363 | 15:7277661 | 70364 | 15:7278217 |
| 70411 | 15:8492046 | 70427 | 15:9014277 | 70429 | 15:9031126 | 70432 | 15:9102618 | 70438 | 15:9169770 |
| 70439 | 15:9233443 | 70471 | 15:10068806 | 70472 | 15:10075553 | 70474 | 15:10079990 | 70475 | 15:10080765 |
| 70476 | 15:10081644 | 70511 | 15:11396612 | 70557 | 15:13243732 | 70596 | 15:14363124 | 70609 | 15:14575841 |
| 70610 | 15:14594338 | 70629 | 15:15205635 | 70630 | 15:15239992 | 70707 | 15:16376589 | 70708 | 15:16439066 |
| 70735 | 15:17085252 | 70738 | 15:17150531 | 70745 | 15:17184652 | 70765 | 15:17609663 | 70797 | 15:18443747 |
| 70805 | 15:18738117 | 70819 | 15:19107499 | 70839 | 15:19655124 | 70859 | 15:20139691 | 70869 | 15:20440646 |
| 70890 | 15:20723501 | 70945 | 15:22143335 | 70975 | 15:22778474 | 70976 | 15:22778563 | 70977 | 15:22779768 |
| 70984 | 15:22928839 | 70990 | 15:23127718 | 70993 | 15:23186412 | 71073 | 15:24244322 | 71098 | 15:24733535 |
| 71100 | 15:24758009 | 71125 | 15:25277078 | 71134 | 15:25472864 | 71147 | 15:25791312 | 71158 | 15:25955785 |
| 71179 | 15:26356076 | 71196 | 15:26746546 | 71208 | 15:27116571 | 71241 | 15:27939885 | 71250 | 15:28057977 |
| 71255 | 15:28119260 | 71281 | 15:28579106 | 71361 | 15:30204684 | 71411 | 15:31423842 | 71480 | 15:32908026 |
| 71484 | 15:32949060 | 71520 | 15:33787224 | 71547 | 15:34234555 | 71557 | 15:34409813 | 71575 | 15:34768501 |
| 71595 | 15:35125952 | 71629 | 15:36004891 | 71630 | 15:36008307 | 71631 | 15:36026492 | 71648 | 15:36401889 |
| 71659 | 15:36632711 | 71691 | 15:37336930 | 71694 | 15:37400581 | 71695 | 15:37400733 | 71696 | 15:37402318 |
| 71697 | 15:37402363 | 71698 | 15:37402682 | 71699 | 15:37403163 | 71700 | 15:37405162 | 71701 | 15:37406604 |
| 71702 | 15:37408508 | 71726 | 15:38124244 | 71729 | 15:38238774 | 71747 | 15:38627437 | 71754 | 15:38756627 |
| 71765 | 15:39027306 | 71774 | 15:39310122 | 71779 | 15:39360198 | 71788 | 15:39612413 | 71831 | 15:40620626 |
| 71848 | 15:41008572 | 71872 | 15:41577758 | 71873 | 15:41586379 | 71894 | 15:41925488 | 71937 | 15:42825209 |
| 71945 | 15:42913923 | 71990 | 15:43478979 | 72010 | 15:43778894 | 72015 | 15:43842403 | 72025 | 15:43940904 |
| 72031 | 15:44171870 | 72055 | 15:44625080 | 72119 | 15:46158689 | 72132 | 15:46492762 | 72164 | 15:46744389 |
| 72165 | 15:46777715 | 72167 | 15:46785999 | 72168 | 15:46790581 | 72169 | 15:46804156 | 72170 | 15:46852906 |
| 72171 | 15:46854891 | 72175 | 15:46873315 | 72219 | 15:47951518 | 72223 | 15:48016378 | 72242 | 15:48211767 |
| 72259 | 15:48584212 | 72288 | 15:49705651 | 72400 | 15:52067358 | 72432 | 15:52906987 | 72450 | 15:53308730 |
| 72455 | 15:53312462 | 72514 | 15:54171179 | 72571 | 15:55496596 | 72580 | 15:55679447 | 72584 | 15:55794756 |
| 72629 | 15:56617358 | 72704 | 15:57721772 | 72710 | 15:57822336 | 72753 | 15:58596845 | 72786 | 15:59497090 |
| 72810 | 15:60299723 | 72821 | 15:60544990 | 72828 | 15:60665356 | 72838 | 15:61116189 | 72841 | 15:61144336 |
| 72844 | 15:61187610 | 72847 | 15:61230749 | 72848 | 15:61235200 | 72883 | 15:61807140 | 72897 | 15:62087105 |
| 72904 | 15:62399301 | 72913 | 15:62562520 | 72945 | 15:63169519 | 72956 | 15:63484094 | 72978 | 15:63866432 |
| 72980 | 15:63956384 | 72984 | 15:63969563 | 73010 | 15:64570035 | 73011 | 15:64573599 | 73012 | 15:64581068 |
| 73013 | 15:64581832 | 73014 | 15:64581860 | 73015 | 15:64587577 | 73016 | 15:64587694 | 73017 | 15:64587726 |
| 73018 | 15:64587727 | 73019 | 15:64587894 | 73032 | 15:64805974 | 73034 | 15:64822114 | 73036 | 15:64828957 |
| 73037 | 15:64829763 | 73055 | 15:65133696 | 73057 | 15:65176452 | 73068 | 15:65338322 | 73110 | 15:66198995 |
| 73133 | 15:66626921 | 73136 | 15:66654107 | 73137 | 15:66659219 | 73157 | 15:67252520 | 73183 | 15:67982185 |
| 73232 | 15:69401513 | 73297 | 15:71439330 | 73316 | 15:71924774 | 73318 | 15:71954992 | 73320 | 15:71975292 |
| 73324 | 15:72049215 | 73432 | 15:74395188 | 73433 | 15:74409395 | 73453 | 15:74803075 | 73462 | 15:75064377 |
| 73463 | 15:75065891 | 73464 | 15:75071311 | 73469 | 15:75183095 | 73471 | 15:75207042 | 73478 | 15:75433003 |
| 73581 | 15:77653525 | 73585 | 15:77702400 | 73588 | 15:77785862 | 73593 | 15:77886217 | 73647 | 15:78764970 |
| 73658 | 15:79089495 | 73691 | 15:80200231 | 73697 | 15:80308461 | 73708 | 15:80425465 | 73780 | 15:81982872 |
| 73833 | 15:83017252 | 73857 | 15:83501029 | 73858 | 15:83507518 | 73859 | 15:83514913 | 73860 | 15:83519848 |
| 73861 | 15:83524365 | 73863 | 15:83529608 | 73885 | 15:84090905 | 73971 | 16:1756551 | 74018 | 16:2807132 |
| 74022 | 16:2880813 | 74046 | 16:3278886 | 74067 | 16:3645905 | 74074 | 16:3832226 | 74078 | 16:3983617 |
| 74085 | 16:4041201 | 74113 | 16:4472306 | 74116 | 16:4488724 | 74134 | 16:5011968 | 74222 | 16:7875660 |
| 74227 | 16:8061836 | 74247 | 16:8833166 | 74264 | 16:9480615 | 74299 | 16:10761697 | 74367 | 16:12564264 |
| 74370 | 16:12610513 | 74376 | 16:12712264 | 74380 | 16:12809176 | 74416 | 16:13500639 | 74493 | 16:15596092 |
| 74509 | 16:16114051 | 74543 | 16:17341478 | 74577 | 16:18497037 | 74680 | 16:21073857 | 74697 | 16:21482215 |
| 74705 | 16:21676658 | 74716 | 16:22006522 | 74717 | 16:22052486 | 74721 | 16:22158028 | 74731 | 16:22445521 |
| 74735 | 16:22522151 | 74736 | 16:22527368 | 74784 | 16:23721556 | 74795 | 16:23949095 | 74810 | 16:24332233 |
| 74873 | 16:25194415 | 74886 | 16:25230576 | 74895 | 16:25259731 | 74896 | 16:25267299 | 74912 | 16:25869035 |
| 74914 | 16:25919271 | 74922 | 16:26068625 | 74962 | 16:26927299 | 75005 | 16:28019627 | 75093 | 16:30731403 |
| 75115 | 16:31224822 | 75116 | 16:31245895 | 75135 | 16:31715125 | 75141 | 16:31812156 | 75192 | 16:33108294 |
| 75193 | 16:33145311 | 75196 | 16:33256074 | 75208 | 16:33505042 | 75221 | 16:33889094 | 75227 | 16:34042467 |
| 75228 | 16:34043548 | 75232 | 16:34106781 | 75248 | 16:34589155 | 75329 | 16:36525688 | 75352 | 16:37160322 |
| 75435 | 16:39120749 | 75447 | 16:39536783 | 75474 | 16:40415228 | 75484 | 16:40686277 | 75519 | 16:41883664 |
| 75545 | 16:42004893 | 75548 | 16:42055479 | 75562 | 16:42802530 | 75565 | 16:42843655 | 75640 | 16:46097209 |
| 75644 | 16:46156095 | 75677 | 16:46962612 | 75715 | 16:47752936 | 75724 | 16:48024037 | 75725 | 16:48025956 |
| 75726 | 16:48033049 | 75727 | 16:48034263 | 75729 | 16:48067867 | 75734 | 16:48186793 | 75735 | 16:48187331 |
| 75736 | 16:48187489 | 75737 | 16:48223403 | 75738 | 16:48230117 | 75739 | 16:48232464 | 75751 | 16:48528203 |
| 75754 | 16:48566545 | 75755 | 16:48591914 | 75771 | 16:48864424 | 75776 | 16:49014011 | 75786 | 16:49264493 |
| 75852 | 16:51207748 | 75916 | 16:52579557 | 76051 | 16:56128576 | 76058 | 16:56235648 | 76083 | 16:56653049 |
| 76110 | 16:57538081 | 76150 | 16:58399171 | 76164 | 16:58621735 | 76178 | 16:58963242 | 76182 | 16:59073877 |
| 76184 | 16:59075986 | 76190 | 16:59245993 | 76192 | 16:59265813 | 76193 | 16:59266170 | 76200 | 16:59328729 |
| 76205 | 16:59411723 | 76226 | 16:60040160 | 76243 | 16:60336822 | 76259 | 16:60792834 | 76289 | 16:61435887 |
| 76345 | 16:62739421 | 76365 | 16:63159271 | 76368 | 16:63219362 | 76383 | 16:63498892 | 76385 | 16:63554652 |
| 76398 | 16:63842771 | 76400 | 16:63858162 | 76411 | 16:64136818 | 76424 | 16:64408574 | 76435 | 16:64563350 |
| 76440 | 16:64632519 | 76442 | 16:64637819 | 76443 | 16:64637824 | 76444 | 16:64637826 | 76453 | 16:64811600 |
| 76524 | 16:66273725 | 76529 | 16:66428578 | 76542 | 16:66625242 | 76552 | 16:66834448 | 76585 | 16:67467822 |
| 76587 | 16:67495360 | 76590 | 16:67506416 | 76593 | 16:67552727 | 76603 | 16:67685441 | 76635 | 16:68623796 |
| 76640 | 16:68718917 | 76642 | 16:68746747 | 76646 | 16:68890172 | 76655 | 16:69079413 | 76691 | 16:69777086 |
| 76693 | 16:69813037 | 76701 | 16:70045391 | 76717 | 16:70339833 | 76729 | 16:70576773 | 76748 | 16:70946593 |
| 76774 | 16:71760780 | 76788 | 16:72102560 | 76825 | 16:72662418 | 76830 | 16:72687591 | 76842 | 16:72970330 |
| 76861 | 16:73473194 | 76864 | 16:73513959 | 76895 | 16:73873564 | 76951 | 16:75067479 | 76972 | 16:75350380 |
| 76990 | 16:75714163 | 77032 | 16:76538771 | 77064 | 16:77153817 | 77099 | 16:77965851 | 77102 | 16:78019437 |
| 77132 | 16:78710736 | 77152 | 16:78992583 | 77166 | 16:79102626 | 77238 | 16:80715002 | 77241 | 16:80886197 |
| 77246 | 17:111106 | 77250 | 17:171222 | 77251 | 17:172400 | 77253 | 17:305126 | 77262 | 17:486383 |
| 77374 | 17:3335273 | 77379 | 17:3441488 | 77422 | 17:4260319 | 77443 | 17:4584704 | 77446 | 17:4621398 |
| 77452 | 17:4717447 | 77469 | 17:5097696 | 77470 | 17:5097857 | 77489 | 17:5622055 | 77510 | 17:6077823 |
| 77557 | 17:6872378 | 77579 | 17:7317589 | 77580 | 17:7338578 | 77582 | 17:7342145 | 77586 | 17:7478372 |
| 77606 | 17:7876357 | 77608 | 17:7883935 | 77619 | 17:8132536 | 77638 | 17:8600944 | 77642 | 17:8668787 |
| 77704 | 17:10109146 | 77716 | 17:10263523 | 77725 | 17:10597515 | 77742 | 17:11173860 | 77747 | 17:11293045 |
| 77753 | 17:11395090 | 77762 | 17:11714108 | 77763 | 17:11715058 | 77770 | 17:11817054 | 77787 | 17:12195499 |
| 77788 | 17:12195568 | 77789 | 17:12195662 | 77790 | 17:12196276 | 77791 | 17:12196708 | 77805 | 17:12486159 |
| 77808 | 17:12628635 | 77906 | 17:14816935 | 77909 | 17:14863506 | 77931 | 17:15328888 | 77949 | 17:15663502 |
| 77958 | 17:15910083 | 77975 | 17:16368625 | 78009 | 17:17274244 | 78010 | 17:17279868 | 78011 | 17:17280788 |
| 78012 | 17:17280990 | 78013 | 17:17281225 | 78024 | 17:17411908 | 78039 | 17:17775173 | 78082 | 17:18818243 |
| 78086 | 17:18869685 | 78105 | 17:19223651 | 78106 | 17:19245275 | 78109 | 17:19292348 | 78110 | 17:19295355 |
| 78111 | 17:19295356 | 78112 | 17:19295499 | 78113 | 17:19295504 | 78133 | 17:19689334 | 78153 | 17:20218072 |
| 78173 | 17:20920601 | 78177 | 17:21089322 | 78195 | 17:21650017 | 78203 | 17:21824364 | 78259 | 17:24078894 |
| 78278 | 17:24557966 | 78308 | 17:25485693 | 78320 | 17:25835294 | 78333 | 17:26208169 | 78336 | 17:26275689 |
| 78338 | 17:26313864 | 78347 | 17:26586857 | 78355 | 17:26669513 | 78379 | 17:27150917 | 78417 | 17:28123761 |
| 78429 | 17:28336908 | 78457 | 17:29248479 | 78514 | 17:30796206 | 78543 | 17:31545528 | 78556 | 17:31865010 |
| 78558 | 17:31877966 | 78579 | 17:32478146 | 78583 | 17:32532692 | 78596 | 17:32813643 | 78625 | 17:33559323 |
| 78633 | 17:33630931 | 78661 | 17:34398154 | 78696 | 17:35542521 | 78722 | 17:36254116 | 78747 | 17:37178207 |
| 78773 | 17:38080956 | 78783 | 17:38278802 | 78789 | 17:38562984 | 78797 | 17:38814783 | 78824 | 17:39676479 |
| 78825 | 17:39677255 | 78826 | 17:39679094 | 78832 | 17:39827471 | 78833 | 17:39828115 | 78834 | 17:39828525 |
| 78836 | 17:39840626 | 78838 | 17:39849566 | 78839 | 17:39854144 | 78840 | 17:39858951 | 78843 | 17:39882893 |
| 78844 | 17:39883222 | 78845 | 17:39885556 | 78846 | 17:39886164 | 78847 | 17:39887756 | 78848 | 17:39893701 |
| 78849 | 17:39894975 | 78850 | 17:39896327 | 78851 | 17:39896972 | 78877 | 17:40559838 | 78880 | 17:40569673 |
| 78881 | 17:40569857 | 78882 | 17:40570434 | 78885 | 17:40597210 | 78889 | 17:40687599 | 78891 | 17:40702874 |
| 78893 | 17:40727620 | 78924 | 17:41582600 | 78993 | 17:43272612 | 78996 | 17:43327605 | 79034 | 17:44279469 |
| 79079 | 17:45388796 | 79090 | 17:45567473 | 79117 | 17:46182697 | 79173 | 17:47495174 | 79183 | 17:47822968 |
| 79184 | 17:47826553 | 79203 | 17:48325513 | 79216 | 17:48653000 | 79227 | 17:48892420 | 79233 | 17:49128208 |
| 79234 | 17:49131387 | 79272 | 17:49921719 | 79309 | 17:50714394 | 79310 | 17:50720459 | 79311 | 17:50722838 |
| 79312 | 17:50723471 | 79313 | 17:50727878 | 79314 | 17:50730200 | 79315 | 17:50731825 | 79317 | 17:50814958 |
| 79357 | 17:51667657 | 79381 | 17:52060992 | 79385 | 17:52081616 | 79386 | 17:52082865 | 79387 | 17:52087837 |
| 79388 | 17:52087901 | 79424 | 17:53278122 | 79462 | 17:54135006 | 79538 | 17:56024288 | 79550 | 17:56299499 |
| 79555 | 17:56435818 | 79572 | 17:57052924 | 79586 | 17:57342574 | 79605 | 17:57757425 | 79607 | 17:57774071 |
| 79611 | 17:57814505 | 79628 | 17:58146798 | 79636 | 17:58314278 | 79637 | 17:58317198 | 79638 | 17:58321497 |
| 79639 | 17:58322279 | 79640 | 17:58323397 | 79641 | 17:58324863 | 79642 | 17:58325379 | 79643 | 17:58325583 |
| 79644 | 17:58325642 | 79645 | 17:58325662 | 79646 | 17:58325692 | 79647 | 17:58325868 | 79648 | 17:58325926 |
| 79649 | 17:58326148 | 79650 | 17:58330227 | 79651 | 17:58331401 | 79653 | 17:58334167 | 79654 | 17:58335083 |
| 79655 | 17:58335597 | 79656 | 17:58337576 | 79657 | 17:58338335 | 79681 | 17:59019311 | 79699 | 17:59378547 |
| 79701 | 17:59392366 | 79712 | 17:59683258 | 79747 | 17:60329105 | 79749 | 17:60358707 | 79757 | 17:60444808 |
| 79798 | 17:61041921 | 79820 | 17:61336784 | 79824 | 17:61432513 | 79831 | 17:61511820 | 79893 | 17:63198489 |
| 79977 | 17:64661316 | 79989 | 17:64973779 | 79994 | 17:65038744 | 80026 | 17:65697499 | 80041 | 17:65972365 |
| 80051 | 17:66165602 | 80052 | 17:66169310 | 80053 | 17:66170354 | 80054 | 17:66171241 | 80055 | 17:66171742 |
| 80056 | 17:66172830 | 80071 | 17:66374177 | 80113 | 17:67428489 | 80128 | 17:67720645 | 80131 | 17:67842378 |
| 80139 | 17:68113867 | 80189 | 17:69059007 | 80213 | 17:69580013 | 80223 | 17:69757640 | 80227 | 17:69882555 |
| 80246 | 17:70318401 | 80276 | 17:70659081 | 80282 | 17:70929780 | 80318 | 17:71539730 | 80327 | 17:71761848 |
| 80331 | 17:71853596 | 80332 | 17:71855169 | 80333 | 17:71859405 | 80335 | 17:71900536 | 80406 | 18:712517 |
| 80420 | 18:990363 | 80449 | 18:1484040 | 80458 | 18:1637197 | 80473 | 18:1743436 | 80501 | 18:1802242 |
| 80511 | 18:2001978 | 80533 | 18:2557098 | 80550 | 18:2832635 | 80557 | 18:2935768 | 80565 | 18:3102978 |
| 80575 | 18:3297749 | 80603 | 18:4036722 | 80612 | 18:4219952 | 80613 | 18:4226405 | 80620 | 18:4439946 |
| 80628 | 18:4565913 | 80644 | 18:4871817 | 80669 | 18:5415676 | 80670 | 18:5415711 | 80671 | 18:5416566 |
| 80712 | 18:6220320 | 80723 | 18:6398640 | 80725 | 18:6407051 | 80726 | 18:6407842 | 80727 | 18:6407856 |
| 80728 | 18:6408160 | 80729 | 18:6409137 | 80730 | 18:6409528 | 80748 | 18:6759745 | 80753 | 18:6859408 |
| 80770 | 18:7273399 | 80793 | 18:7769343 | 80802 | 18:7953745 | 80851 | 18:8931926 | 80872 | 18:9156689 |
| 80884 | 18:9413940 | 80885 | 18:9414216 | 80909 | 18:9827683 | 80924 | 18:10096838 | 80935 | 18:10422385 |
| 80980 | 18:11195149 | 81051 | 18:13077929 | 81083 | 18:13931354 | 81085 | 18:13962063 | 81086 | 18:13962211 |
| 81094 | 18:14131401 | 81128 | 18:15337353 | 81175 | 18:16420426 | 81195 | 18:16937183 | 81202 | 18:17081163 |
| 81213 | 18:17191494 | 81240 | 18:17819065 | 81249 | 18:18041884 | 81257 | 18:18196329 | 81262 | 18:18274003 |
| 81263 | 18:18274911 | 81264 | 18:18279929 | 81265 | 18:18284551 | 81266 | 18:18285334 | 81268 | 18:18287100 |
| 81270 | 18:18297472 | 81271 | 18:18300144 | 81289 | 18:18654230 | 81301 | 18:18883470 | 81336 | 18:19805409 |
| 81365 | 18:20391312 | 81369 | 18:20532028 | 81370 | 18:20581067 | 81371 | 18:20586213 | 81372 | 18:20591204 |
| 81373 | 18:20594221 | 81383 | 18:20851094 | 81434 | 18:22045866 | 81499 | 18:23267678 | 81519 | 18:23730169 |
| 81533 | 18:24068173 | 81584 | 18:25002158 | 81600 | 18:25402965 | 81616 | 18:25722314 | 81657 | 18:26620395 |
| 81660 | 18:26643719 | 81667 | 18:26781973 | 81699 | 18:27670793 | 81722 | 18:28212436 | 81754 | 18:29332445 |
| 81762 | 18:29394581 | 81775 | 18:29615496 | 81779 | 18:29751573 | 81780 | 18:29770581 | 81825 | 18:31291350 |
| 81850 | 18:31971311 | 81863 | 18:32267474 | 81869 | 18:32301839 | 81874 | 18:32426539 | 81917 | 18:33371652 |
| 81918 | 18:33381630 | 81919 | 18:33381846 | 81921 | 18:33389064 | 81930 | 18:33546416 | 81931 | 18:33559158 |
| 81956 | 18:34226859 | 82000 | 18:35324050 | 82017 | 18:35722943 | 82090 | 18:37440845 | 82096 | 18:37511943 |
| 82111 | 18:37857222 | 82117 | 18:37902748 | 82162 | 18:38995251 | 82167 | 18:39072678 | 82201 | 18:39856656 |
| 82211 | 18:40078593 | 82214 | 18:40135404 | 82268 | 18:41177791 | 82300 | 18:41952487 | 82332 | 18:42484052 |
| 82374 | 18:43224250 | 82381 | 18:43359712 | 82384 | 18:43378123 | 82402 | 18:43762058 | 82434 | 18:44688799 |
| 82436 | 18:44703246 | 82492 | 18:46177257 | 82495 | 18:46269611 | 82566 | 18:46759650 | 82608 | 18:47374533 |
| 82675 | 18:48506540 | 82731 | 18:49938198 | 82779 | 18:51259647 | 82786 | 18:51451371 | 82787 | 18:51452185 |
| 82788 | 18:51455635 | 82792 | 18:51522776 | 82795 | 18:51579528 | 82817 | 18:51820303 | 82847 | 18:52482506 |
| 82860 | 18:52950856 | 82873 | 18:53350033 | 82882 | 18:53554813 | 82896 | 18:53718739 | 82916 | 18:54039068 |
| 82934 | 18:54414979 | 82973 | 18:55042223 | 83009 | 18:55891884 | 83050 | 18:56619470 | 83056 | 18:56702388 |
| 83061 | 18:56777892 | 83063 | 18:56780574 | 83064 | 18:56781239 | 83065 | 18:56788137 | 83066 | 18:56788537 |
| 83067 | 18:56790131 | 83068 | 18:56793472 | 83069 | 18:56793653 | 83070 | 18:56802310 | 83071 | 18:56802366 |
| 83072 | 18:56802408 | 83073 | 18:56803155 | 83074 | 18:56805465 | 83075 | 18:56805669 | 83076 | 18:56805763 |
| 83077 | 18:56805884 | 83078 | 18:56805908 | 83079 | 18:56805969 | 83080 | 18:56806452 | 83081 | 18:56806522 |
| 83082 | 18:56806817 | 83083 | 18:56807069 | 83084 | 18:56807143 | 83085 | 18:56807200 | 83086 | 18:56808520 |
| 83087 | 18:56808693 | 83088 | 18:56808739 | 83089 | 18:56809175 | 83090 | 18:56809229 | 83091 | 18:56809274 |
| 83092 | 18:56809314 | 83093 | 18:56809399 | 83094 | 18:56811400 | 83095 | 18:56816836 | 83096 | 18:56817529 |
| 83097 | 18:56817530 | 83098 | 18:56817556 | 83099 | 18:56817983 | 83100 | 18:56819032 | 83102 | 18:56819579 |
| 83103 | 18:56820099 | 83104 | 18:56820288 | 83105 | 18:56820485 | 83106 | 18:56821287 | 83129 | 18:57480828 |
| 83131 | 18:57489304 | 83132 | 18:57496016 | 83135 | 18:57545249 | 83136 | 18:57549761 | 83137 | 18:57559274 |
| 83154 | 18:57826436 | 83155 | 18:57826437 | 83164 | 18:57929781 | 83230 | 18:58590500 | 83236 | 18:58874702 |
| 83262 | 18:60020922 | 83277 | 18:60611535 | 83278 | 18:60661061 | 83292 | 18:61082028 | 83295 | 18:61224849 |
| 83317 | 18:61691526 | 83377 | 18:62817960 | 83416 | 18:63491911 | 83425 | 18:63743739 | 83431 | 18:63828080 |
| 83432 | 18:63835095 | 83437 | 18:63865767 | 83560 | 18:65350149 | 83571 | 18:65487247 | 83589 | 19:153194 |
| 83605 | 19:905203 | 83670 | 19:3029129 | 83693 | 19:3751571 | 83731 | 19:4866448 | 83744 | 19:5165734 |
| 83761 | 19:5576963 | 83775 | 19:5960251 | 83792 | 19:6497947 | 83798 | 19:6642039 | 83815 | 19:6924319 |
| 83883 | 19:8123606 | 83898 | 19:8310681 | 83901 | 19:8332089 | 83902 | 19:8333798 | 83903 | 19:8344332 |
| 83904 | 19:8344354 | 83905 | 19:8356008 | 83906 | 19:8366630 | 83907 | 19:8366749 | 83916 | 19:8476307 |
| 83918 | 19:8521460 | 83930 | 19:8822288 | 83958 | 19:9228505 | 83982 | 19:9886007 | 83992 | 19:10035802 |
| 83998 | 19:10157460 | 83999 | 19:10157552 | 84100 | 19:12546324 | 84101 | 19:12551345 | 84104 | 19:12587276 |
| 84139 | 19:13390721 | 84143 | 19:13523234 | 84144 | 19:13523312 | 84145 | 19:13523394 | 84146 | 19:13523599 |
| 84147 | 19:13523669 | 84148 | 19:13523920 | 84149 | 19:13524217 | 84150 | 19:13524534 | 84151 | 19:13524690 |
| 84152 | 19:13525738 | 84191 | 19:14079144 | 84213 | 19:14542746 | 84214 | 19:14547882 | 84228 | 19:14975147 |
| 84229 | 19:14976930 | 84230 | 19:14977190 | 84240 | 19:15153935 | 84243 | 19:15176665 | 84277 | 19:15979571 |
| 84333 | 19:16959557 | 84403 | 19:18344112 | 84406 | 19:18364792 | 84491 | 19:20068707 | 84575 | 19:21354124 |
| 84635 | 19:22407739 | 84683 | 19:23510771 | 84701 | 19:23901688 | 84718 | 19:24121007 | 84738 | 19:24347182 |
| 84800 | 19:25069906 | 84803 | 19:25117527 | 84804 | 19:25118864 | 84812 | 19:25238500 | 84840 | 19:25573317 |
| 84841 | 19:25583861 | 84842 | 19:25635561 | 84880 | 19:26078995 | 84883 | 19:26146723 | 84884 | 19:26158447 |
| 84885 | 19:26162940 | 84886 | 19:26166792 | 84887 | 19:26177724 | 84888 | 19:26180605 | 84899 | 19:26370592 |
| 84901 | 19:26384973 | 84902 | 19:26386173 | 84904 | 19:26386998 | 84905 | 19:26408649 | 84906 | 19:26414194 |
| 84907 | 19:26425088 | 84908 | 19:26428880 | 84910 | 19:26432937 | 84912 | 19:26434534 | 84916 | 19:26440887 |
| 84917 | 19:26442377 | 84918 | 19:26442417 | 84922 | 19:26458156 | 84925 | 19:26462109 | 84928 | 19:26466738 |
| 84930 | 19:26468564 | 84931 | 19:26485885 | 84932 | 19:26489724 | 84936 | 19:26508139 | 84939 | 19:26510614 |
| 84942 | 19:26555053 | 84944 | 19:26575215 | 84945 | 19:26588232 | 84946 | 19:26595700 | 84947 | 19:26595755 |
| 84948 | 19:26599010 | 84950 | 19:26628925 | 84951 | 19:26662321 | 84953 | 19:26675470 | 84954 | 19:26676204 |
| 84956 | 19:26697210 | 84958 | 19:26705636 | 84959 | 19:26707790 | 84960 | 19:26719756 | 84961 | 19:26732472 |
| 84963 | 19:26744245 | 85040 | 19:28793496 | 85083 | 19:29602130 | 85084 | 19:29603381 | 85102 | 19:29967023 |
| 85123 | 19:30488002 | 85130 | 19:30639326 | 85131 | 19:30644348 | 85132 | 19:30645410 | 85133 | 19:30648555 |
| 85134 | 19:30696412 | 85137 | 19:30818025 | 85139 | 19:30826714 | 85167 | 19:31463142 | 85168 | 19:31485672 |
| 85183 | 19:31907703 | 85187 | 19:31968664 | 85207 | 19:32399697 | 85219 | 19:32665116 | 85236 | 19:33046409 |
| 85285 | 19:34025535 | 85395 | 19:35866723 | 85400 | 19:35970366 | 85434 | 19:36329939 | 85487 | 19:37333280 |
| 85581 | 19:40048911 | 85595 | 19:40251758 | 85597 | 19:40279227 | 85615 | 19:40640928 | 85635 | 19:40995967 |
| 85654 | 19:41486757 | 85668 | 19:41704492 | 85669 | 19:41719174 | 85686 | 19:41943823 | 85702 | 19:42161619 |
| 85726 | 19:42580737 | 85734 | 19:42764834 | 85760 | 19:43424335 | 85761 | 19:43424535 | 85762 | 19:43428799 |
| 85763 | 19:43431509 | 85764 | 19:43433116 | 85765 | 19:43435751 | 85766 | 19:43438458 | 85770 | 19:43493558 |
| 85778 | 19:43722064 | 85864 | 19:45668089 | 85868 | 19:45717594 | 85869 | 19:45717924 | 85870 | 19:45717976 |
| 85871 | 19:45717979 | 85872 | 19:45718107 | 85873 | 19:45718307 | 85874 | 19:45718340 | 85875 | 19:45720202 |
| 85876 | 19:45720264 | 85877 | 19:45720488 | 85878 | 19:45720862 | 85879 | 19:45736048 | 85880 | 19:45738664 |
| 85881 | 19:45740485 | 85882 | 19:45741144 | 85883 | 19:45741389 | 85884 | 19:45741952 | 85885 | 19:45742152 |
| 85886 | 19:45742153 | 85894 | 19:45834449 | 85995 | 19:48080018 | 86018 | 19:48469710 | 86020 | 19:48505288 |
| 86052 | 19:49235212 | 86059 | 19:49331336 | 86060 | 19:49332593 | 86117 | 19:50655487 | 86124 | 19:50694652 |
| 86125 | 19:50699155 | 86126 | 19:50703436 | 86127 | 19:50711021 | 86128 | 19:50712302 | 86129 | 19:50714204 |
| 86130 | 19:50715174 | 86131 | 19:50716477 | 86132 | 19:50718075 | 86133 | 19:50724757 | 86134 | 19:50732339 |
| 86135 | 19:50734637 | 86136 | 19:50741020 | 86137 | 19:50772016 | 86210 | 19:51559279 | 86211 | 19:51559359 |
| 86239 | 19:52037026 | 86255 | 19:52551695 | 86317 | 19:53746270 | 86327 | 19:53883618 | 86328 | 19:53883934 |
| 86336 | 19:54027650 | 86340 | 19:54047174 | 86359 | 19:54403366 | 86370 | 19:54621153 | 86373 | 19:54664915 |
| 86374 | 19:54665643 | 86375 | 19:54667304 | 86377 | 19:54672612 | 86392 | 19:54949422 | 86397 | 19:55036335 |
| 86412 | 19:55420287 | 86545 | 19:56454067 | 86547 | 19:56460077 | 86568 | 19:56726108 | 86584 | 19:57037210 |
| 86601 | 19:57419304 | 86605 | 19:57490381 | 86606 | 19:57508171 | 86615 | 19:57670291 | 86624 | 19:57908956 |
| 86637 | 19:58189519 | 86654 | 19:58502690 | 86661 | 19:58677753 | 86662 | 19:58689620 | 86691 | 19:59301702 |
| 86694 | 19:59353477 | 86714 | 19:59622681 | 86730 | 19:59843660 | 86735 | 19:59905716 | 86755 | 19:60165330 |
| 86757 | 19:60175972 | 86758 | 19:60176459 | 86760 | 19:60192077 | 86784 | 19:60532635 | 86793 | 19:60643681 |
| 86797 | 19:60699603 | 86803 | 19:60819634 | 86816 | 19:61121915 | 86853 | 19:61536597 | 86863 | 19:61642528 |
| 86864 | 19:61642559 | 86875 | 19:61831896 | 86896 | 19:62236608 | 86901 | 19:62290414 | 86906 | 19:62421264 |
| 86921 | 19:62751964 | 86923 | 19:62757779 | 86937 | 19:62914703 | 86971 | 20:465779 | 86984 | 20:581428 |
| 86985 | 20:583434 | 87026 | 20:1587207 | 87034 | 20:1637477 | 87046 | 20:1840861 | 87049 | 20:1872065 |
| 87081 | 20:2302262 | 87098 | 20:2623153 | 87109 | 20:3049545 | 87110 | 20:3050301 | 87111 | 20:3052518 |
| 87112 | 20:3056100 | 87113 | 20:3056394 | 87114 | 20:3061100 | 87115 | 20:3061457 | 87116 | 20:3062741 |
| 87117 | 20:3064116 | 87118 | 20:3064279 | 87119 | 20:3065002 | 87120 | 20:3065571 | 87121 | 20:3065997 |
| 87185 | 20:4419534 | 87443 | 20:5918697 | 87517 | 20:7068652 | 87549 | 20:7706966 | 87560 | 20:7984208 |
| 87598 | 20:8931603 | 87671 | 20:10580364 | 87672 | 20:10581845 | 87697 | 20:11193697 | 87724 | 20:11666353 |
| 87731 | 20:11849546 | 87737 | 20:12016234 | 87780 | 20:12743838 | 87791 | 20:13014120 | 87797 | 20:13145133 |
| 87811 | 20:13530219 | 87817 | 20:13767878 | 87836 | 20:14332360 | 87852 | 20:15273040 | 87864 | 20:15564869 |
| 87875 | 20:15869027 | 87906 | 20:16621914 | 87909 | 20:16650192 | 87961 | 20:18107968 | 87991 | 20:19026521 |
| 87993 | 20:19083370 | 88011 | 20:19606439 | 88019 | 20:19755587 | 88078 | 20:21474908 | 88083 | 20:21584291 |
| 88117 | 20:22063416 | 88191 | 20:22598687 | 88200 | 20:22757553 | 88209 | 20:22931278 | 88259 | 20:23864507 |
| 88277 | 20:23991980 | 88323 | 20:25147490 | 88359 | 20:26491689 | 88360 | 20:26496401 | 88361 | 20:26502059 |
| 88362 | 20:26504123 | 88363 | 20:26514971 | 88364 | 20:26518907 | 88366 | 20:26522200 | 88406 | 20:27651444 |
| 88407 | 20:27665296 | 88420 | 20:27980203 | 88434 | 20:28277863 | 88467 | 20:28789560 | 88482 | 20:29236907 |
| 88526 | 20:30411387 | 88535 | 20:30681003 | 88568 | 20:31716095 | 88572 | 20:31786758 | 88574 | 20:31871902 |
| 88685 | 20:34576290 | 88806 | 20:37374849 | 88817 | 20:37489422 | 88837 | 20:37861824 | 88857 | 20:38404986 |
| 88885 | 20:39228116 | 88889 | 20:39268509 | 88899 | 20:39457698 | 88902 | 20:39467997 | 88907 | 20:39535650 |
| 88913 | 20:39565695 | 88931 | 20:39982244 | 88942 | 20:40219777 | 88959 | 20:40746359 | 88976 | 20:41050966 |
| 89002 | 20:41628336 | 89045 | 20:42462021 | 89117 | 20:44485586 | 89140 | 20:45144462 | 89147 | 20:45457044 |
| 89148 | 20:45547831 | 89150 | 20:45567543 | 89154 | 20:45642750 | 89156 | 20:45675568 | 89158 | 20:45694391 |
| 89174 | 20:46029522 | 89175 | 20:46040337 | 89176 | 20:46040344 | 89177 | 20:46043226 | 89189 | 20:46349920 |
| 89197 | 20:46707839 | 89206 | 20:47136685 | 89214 | 20:47190891 | 89228 | 20:47736047 | 89277 | 20:49126008 |
| 89278 | 20:49182818 | 89301 | 20:49941637 | 89316 | 20:50311371 | 89348 | 20:51482831 | 89384 | 20:52684364 |
| 89407 | 20:53375334 | 89412 | 20:53445710 | 89417 | 20:53542795 | 89468 | 20:55149259 | 89470 | 20:55168267 |
| 89471 | 20:55169154 | 89474 | 20:55188763 | 89476 | 20:55206284 | 89477 | 20:55207791 | 89481 | 20:55321910 |
| 89488 | 20:55507243 | 89626 | 20:58393406 | 89647 | 20:58770472 | 89651 | 20:58809259 | 89694 | 20:59879577 |
| 89697 | 20:59895328 | 89741 | 20:61269332 | 89769 | 20:61983565 | 89777 | 20:62202768 | 89791 | 20:62523219 |
| 89834 | 20:63451088 | 89878 | 20:64194501 | 89901 | 20:64754306 | 89915 | 20:65165202 | 89925 | 20:65480230 |
| 89931 | 20:65608403 | 89992 | 20:66984199 | 90045 | 20:68049361 | 90056 | 20:68247302 | 90060 | 20:68312623 |
| 90065 | 20:68359426 | 90126 | 20:69498583 | 90156 | 20:70136618 | 90175 | 20:70711203 | 90290 | 21:560149 |
| 90307 | 21:1776376 | 90351 | 21:3330534 | 90366 | 21:3866539 | 90394 | 21:4290924 | 90412 | 21:4894437 |
| 90448 | 21:5848777 | 90485 | 21:7043405 | 90502 | 21:7479548 | 90518 | 21:7765157 | 90533 | 21:8066355 |
| 90536 | 21:8077420 | 90537 | 21:8084411 | 90539 | 21:8091687 | 90540 | 21:8093756 | 90541 | 21:8096108 |
| 90544 | 21:8098084 | 90545 | 21:8100095 | 90547 | 21:8121506 | 90548 | 21:8123911 | 90550 | 21:8132548 |
| 90564 | 21:8374844 | 90580 | 21:8819472 | 90593 | 21:9141650 | 90611 | 21:9612990 | 90614 | 21:9635812 |
| 90615 | 21:9642385 | 90658 | 21:10706242 | 90672 | 21:10964762 | 90688 | 21:11397271 | 90715 | 21:12027109 |
| 90738 | 21:12572473 | 90764 | 21:13059367 | 90801 | 21:13974032 | 90810 | 21:14159859 | 90836 | 21:14800811 |
| 90842 | 21:14976378 | 90881 | 21:15763479 | 90898 | 21:16325852 | 90899 | 21:16327666 | 90900 | 21:16352286 |
| 90902 | 21:16380714 | 90904 | 21:16401319 | 90907 | 21:16447288 | 90937 | 21:16900535 | 90938 | 21:16964578 |
| 90960 | 21:17491219 | 90980 | 21:18041395 | 91095 | 21:19926364 | 91108 | 21:20273386 | 91145 | 21:20915737 |
| 91166 | 21:21373080 | 91192 | 21:21922090 | 91199 | 21:22093113 | 91212 | 21:22196208 | 91297 | 21:24454479 |
| 91302 | 21:24572190 | 91312 | 21:24911518 | 91403 | 21:27617328 | 91405 | 21:27709157 | 91450 | 21:29090583 |
| 91467 | 21:29574040 | 91468 | 21:29585741 | 91469 | 21:29663335 | 91474 | 21:29793976 | 91529 | 21:31234804 |
| 91619 | 21:33320463 | 91621 | 21:33351272 | 91625 | 21:33376637 | 91660 | 21:33757910 | 91681 | 21:34100944 |
| 91710 | 21:34503640 | 91721 | 21:34672141 | 91744 | 21:35184835 | 91759 | 21:35593323 | 91773 | 21:35773499 |
| 91788 | 21:36015961 | 91795 | 21:36164785 | 91824 | 21:36934454 | 91828 | 21:37113932 | 91829 | 21:37118207 |
| 91830 | 21:37118300 | 91917 | 21:39931787 | 91933 | 21:40317765 | 91942 | 21:40547674 | 91996 | 21:41966823 |
| 92040 | 21:42868063 | 92065 | 21:43541273 | 92066 | 21:43549420 | 92077 | 21:43767846 | 92081 | 21:43869821 |
| 92105 | 21:44394987 | 92129 | 21:44779372 | 92150 | 21:45470174 | 92187 | 21:46513102 | 92213 | 21:47280539 |
| 92214 | 21:47282065 | 92215 | 21:47287202 | 92216 | 21:47288654 | 92217 | 21:47288689 | 92218 | 21:47289181 |
| 92220 | 21:47294064 | 92222 | 21:47296827 | 92223 | 21:47297692 | 92224 | 21:47297903 | 92225 | 21:47298236 |
| 92226 | 21:47298622 | 92227 | 21:47298661 | 92228 | 21:47298894 | 92229 | 21:47298942 | 92230 | 21:47299468 |
| 92231 | 21:47299536 | 92232 | 21:47299742 | 92233 | 21:47299950 | 92234 | 21:47300166 | 92236 | 21:47301069 |
| 92237 | 21:47301092 | 92238 | 21:47301705 | 92239 | 21:47301914 | 92240 | 21:47301979 | 92241 | 21:47302160 |
| 92242 | 21:47302405 | 92243 | 21:47302771 | 92244 | 21:47302793 | 92245 | 21:47303150 | 92246 | 21:47303326 |
| 92247 | 21:47303615 | 92248 | 21:47304710 | 92249 | 21:47308349 | 92250 | 21:47309250 | 92251 | 21:47314809 |
| 92253 | 21:47316827 | 92263 | 21:47572280 | 92286 | 21:48277772 | 92318 | 21:49185947 | 92319 | 21:49189566 |
| 92320 | 21:49193705 | 92321 | 21:49194179 | 92323 | 21:49195976 | 92324 | 21:49209645 | 92326 | 21:49218212 |
| 92327 | 21:49229215 | 92328 | 21:49231400 | 92360 | 21:50042557 | 92372 | 21:50467495 | 92407 | 21:51606765 |
| 92424 | 21:52027284 | 92433 | 21:52289877 | 92446 | 21:52553332 | 92471 | 21:53340020 | 92497 | 21:54060629 |
| 92505 | 21:54338413 | 92522 | 21:54761061 | 92523 | 21:54761327 | 92524 | 21:54766467 | 92526 | 21:54773661 |
| 92527 | 21:54779429 | 92528 | 21:54795770 | 92570 | 21:55568962 | 92571 | 21:55571357 | 92575 | 21:55603036 |
| 92577 | 21:55606404 | 92645 | 21:56889237 | 92646 | 21:56907066 | 92724 | 21:57243795 | 92757 | 21:58266418 |
| 92773 | 21:58557250 | 92819 | 21:59483269 | 92840 | 21:59828548 | 92841 | 21:59838076 | 92844 | 21:59872593 |
| 92852 | 21:59962135 | 92853 | 21:59965050 | 92861 | 21:60071151 | 92876 | 21:60290443 | 92897 | 21:60717146 |
| 92945 | 21:61871337 | 92978 | 21:62509823 | 92981 | 21:62565907 | 92997 | 21:62953263 | 93021 | 21:63719399 |
| 93022 | 21:63775744 | 93035 | 21:64020970 | 93043 | 21:64322263 | 93045 | 21:64323466 | 93054 | 21:64458851 |
| 93055 | 21:64468813 | 93061 | 21:64551281 | 93067 | 21:64674944 | 93071 | 21:64702159 | 93075 | 21:64763778 |
| 93111 | 21:65219263 | 93113 | 21:65222945 | 93120 | 21:65350726 | 93122 | 21:65389454 | 93124 | 21:65436920 |
| 93127 | 21:65495121 | 93138 | 21:65745850 | 93203 | 21:67086290 | 93205 | 21:67097791 | 93207 | 21:67137594 |
| 93216 | 21:67459394 | 93217 | 21:67461089 | 93236 | 21:67933900 | 93237 | 21:67933901 | 93267 | 21:68505684 |
| 93284 | 21:68792488 | 93291 | 21:68917772 | 93302 | 21:69177495 | 93307 | 21:69279748 | 93384 | 22:1336082 |
| 93421 | 22:2092658 | 93448 | 22:2744263 | 93451 | 22:2759833 | 93452 | 22:2768731 | 93476 | 22:3271155 |
| 93490 | 22:3549590 | 93492 | 22:3565222 | 93512 | 22:4182249 | 93517 | 22:4255887 | 93535 | 22:4575207 |
| 93596 | 22:5655551 | 93689 | 22:7518330 | 93690 | 22:7526334 | 93700 | 22:7714187 | 93721 | 22:8392537 |
| 93725 | 22:8430070 | 93741 | 22:9021813 | 93749 | 22:9259333 | 93782 | 22:9998563 | 93798 | 22:10114240 |
| 93824 | 22:10733620 | 93845 | 22:11198997 | 93857 | 22:11572590 | 93875 | 22:11958308 | 93895 | 22:12274925 |
| 93927 | 22:12850291 | 93957 | 22:13771925 | 93999 | 22:14949092 | 94000 | 22:14961326 | 94003 | 22:15026827 |
| 94021 | 22:15501897 | 94034 | 22:15667257 | 94037 | 22:15711517 | 94056 | 22:16388677 | 94061 | 22:16514054 |
| 94079 | 22:16739297 | 94121 | 22:17893434 | 94128 | 22:18003077 | 94129 | 22:18033855 | 94139 | 22:18259710 |
| 94140 | 22:18265709 | 94154 | 22:18559489 | 94166 | 22:18849789 | 94167 | 22:18851178 | 94178 | 22:19237876 |
| 94184 | 22:19372836 | 94197 | 22:19837982 | 94198 | 22:19847979 | 94213 | 22:20176939 | 94229 | 22:20643619 |
| 94230 | 22:20646235 | 94241 | 22:20897046 | 94278 | 22:21648235 | 94280 | 22:21664858 | 94296 | 22:21953904 |
| 94303 | 22:22190483 | 94309 | 22:22421295 | 94314 | 22:22604015 | 94324 | 22:22885054 | 94352 | 22:23611781 |
| 94362 | 22:23913434 | 94413 | 22:25649715 | 94420 | 22:25917253 | 94440 | 22:26167092 | 94456 | 22:26660485 |
| 94489 | 22:27621315 | 94505 | 22:28117808 | 94510 | 22:28166101 | 94521 | 22:28401484 | 94530 | 22:28491057 |
| 94568 | 22:29357579 | 94579 | 22:29669451 | 94592 | 22:29900170 | 94598 | 22:30126912 | 94643 | 22:31208133 |
| 94672 | 22:32149285 | 94703 | 22:33059262 | 94715 | 22:33350762 | 94739 | 22:34116071 | 94741 | 22:34136171 |
| 94756 | 22:34692656 | 94757 | 22:34695203 | 94773 | 22:35027110 | 94777 | 22:35094672 | 94779 | 22:35111999 |
| 94789 | 22:35301748 | 94814 | 22:35855583 | 94827 | 22:36060507 | 94829 | 22:36134170 | 94835 | 22:36318773 |
| 94861 | 22:37054760 | 94868 | 22:37235828 | 94896 | 22:37669398 | 94913 | 22:38264582 | 94914 | 22:38283974 |
| 94923 | 22:38466243 | 94937 | 22:38695645 | 94941 | 22:38735989 | 94942 | 22:38740955 | 94960 | 22:39188813 |
| 94989 | 22:39567152 | 94995 | 22:39656245 | 94997 | 22:39682658 | 95055 | 22:41016011 | 95066 | 22:41417903 |
| 95081 | 22:41777356 | 95084 | 22:41792218 | 95085 | 22:41797029 | 95089 | 22:41877932 | 95149 | 22:43286201 |
| 95155 | 22:43386150 | 95178 | 22:44035160 | 95210 | 22:44640940 | 95237 | 22:45552005 | 95247 | 22:45691255 |
| 95263 | 22:45913562 | 95274 | 22:46107748 | 95276 | 22:46144763 | 95297 | 22:46746909 | 95317 | 22:47040045 |
| 95323 | 22:47111303 | 95426 | 22:49820256 | 95433 | 22:49940593 | 95434 | 22:49970190 | 95440 | 22:50067364 |
| 95499 | 22:51502260 | 95500 | 22:51503045 | 95531 | 22:52333167 | 95540 | 22:52556549 | 95558 | 22:52884674 |
| 95565 | 22:52961463 | 95592 | 22:53497101 | 95621 | 22:54011562 | 95672 | 22:55011602 | 95677 | 22:55091224 |
| 95695 | 22:55323748 | 95712 | 22:55563765 | 95758 | 22:56293581 | 95793 | 22:56540115 | 95794 | 22:56540668 |
| 95795 | 22:56542873 | 95796 | 22:56544054 | 95798 | 22:56573939 | 95850 | 22:57521486 | 95882 | 22:57942911 |
| 95894 | 22:58136676 | 95910 | 22:58494061 | 95920 | 22:58678398 | 95922 | 22:58700947 | 95924 | 22:58773917 |
| 95941 | 22:59128229 | 95967 | 22:59629485 | 95973 | 22:59712330 | 95985 | 22:59846886 | 96044 | 23:774985 |
| 96050 | 23:968798 | 96057 | 23:1194189 | 96063 | 23:1325388 | 96069 | 23:1434392 | 96095 | 23:2175010 |
| 96116 | 23:2637687 | 96164 | 23:3563217 | 96210 | 23:4375629 | 96230 | 23:4759481 | 96241 | 23:4872845 |
| 96246 | 23:5028069 | 96328 | 23:6757709 | 96342 | 23:7060820 | 96378 | 23:7879887 | 96380 | 23:7903756 |
| 96398 | 23:8328018 | 96449 | 23:9180909 | 96470 | 23:9852522 | 96499 | 23:10263390 | 96518 | 23:10703356 |
| 96522 | 23:10786602 | 96547 | 23:11354447 | 96568 | 23:11793160 | 96578 | 23:12035181 | 96579 | 23:12038531 |
| 96580 | 23:12039512 | 96582 | 23:12047337 | 96621 | 23:12878298 | 96665 | 23:13769527 | 96690 | 23:14024594 |
| 96691 | 23:14041498 | 96693 | 23:14067842 | 96705 | 23:14437275 | 96707 | 23:14454128 | 96722 | 23:14695783 |
| 96724 | 23:14770597 | 96737 | 23:14938161 | 96742 | 23:15032946 | 96784 | 23:15623072 | 96791 | 23:15668311 |
| 96800 | 23:15846192 | 96807 | 23:16055984 | 96852 | 23:17393860 | 96860 | 23:17578001 | 96864 | 23:17628981 |
| 96866 | 23:17657910 | 96905 | 23:18560341 | 96911 | 23:18766462 | 96916 | 23:18918643 | 96964 | 23:19690687 |
| 96967 | 23:19754433 | 96969 | 23:19762716 | 96978 | 23:19935139 | 96980 | 23:19938929 | 96995 | 23:20172115 |
| 97011 | 23:20449980 | 97015 | 23:20553981 | 97016 | 23:20554380 | 97017 | 23:20555345 | 97018 | 23:20555464 |
| 97019 | 23:20555512 | 97020 | 23:20555877 | 97046 | 23:21266809 | 97053 | 23:21371538 | 97074 | 23:22025892 |
| 97082 | 23:22189816 | 97084 | 23:22253261 | 97099 | 23:22609075 | 97100 | 23:22632358 | 97120 | 23:23494563 |
| 97121 | 23:23495169 | 97122 | 23:23515623 | 97128 | 23:23599173 | 97131 | 23:23663060 | 97133 | 23:23729292 |
| 97144 | 23:23841412 | 97166 | 23:24310745 | 97182 | 23:24584592 | 97220 | 23:24825942 | 97235 | 23:24996122 |
| 97236 | 23:25010458 | 97276 | 23:25343657 | 97294 | 23:25495218 | 97307 | 23:25727838 | 97371 | 23:27168873 |
| 97396 | 23:27404973 | 97400 | 23:27427125 | 97416 | 23:27534819 | 97418 | 23:27545798 | 97509 | 23:28274177 |
| 97515 | 23:28328479 | 97516 | 23:28329217 | 97524 | 23:28449487 | 97546 | 23:28783122 | 97592 | 23:29113119 |
| 97631 | 23:30057510 | 97657 | 23:30485309 | 97658 | 23:30487092 | 97701 | 23:31528534 | 97733 | 23:32121998 |
| 97741 | 23:32302519 | 97742 | 23:32303395 | 97743 | 23:32303732 | 97744 | 23:32304775 | 97745 | 23:32304794 |
| 97746 | 23:32305503 | 97748 | 23:32348823 | 97818 | 23:33768810 | 97821 | 23:33852032 | 97853 | 23:34945913 |
| 97854 | 23:34946818 | 97855 | 23:34946868 | 97856 | 23:34948119 | 97858 | 23:35009819 | 97867 | 23:35427374 |
| 97893 | 23:36297029 | 97899 | 23:36392895 | 97941 | 23:37537593 | 97944 | 23:37550813 | 97949 | 23:37653049 |
| 97985 | 23:38817188 | 97986 | 23:38818425 | 98075 | 23:39639975 | 98079 | 23:39681488 | 98094 | 23:39977867 |
| 98134 | 23:40742268 | 98190 | 23:41726170 | 98215 | 23:42235597 | 98220 | 23:42301353 | 98255 | 23:43110372 |
| 98280 | 23:43675243 | 98300 | 23:44151531 | 98315 | 23:44315474 | 98337 | 23:44789129 | 98433 | 23:46714094 |
| 98477 | 23:47504887 | 98478 | 23:47505469 | 98507 | 23:47898321 | 98510 | 23:47934746 | 98513 | 23:47974902 |
| 98545 | 23:48734960 | 98548 | 23:48756252 | 98551 | 23:48811144 | 98563 | 23:49008700 | 98572 | 23:49166634 |
| 98603 | 23:49594224 | 98626 | 23:49959432 | 98629 | 23:50008283 | 98643 | 23:50262003 | 98652 | 23:50392532 |
| 98685 | 23:51016737 | 98702 | 23:51334319 | 98749 | 23:52192597 | 98751 | 23:52225791 | 98752 | 23:52234534 |
| 98753 | 23:52235518 | 98768 | 24:90983 | 98820 | 24:1186935 | 98822 | 24:1209847 | 98833 | 24:1549261 |
| 98855 | 24:1914090 | 98878 | 24:2417031 | 98920 | 24:3326601 | 98976 | 24:4463662 | 98988 | 24:4937656 |
| 98990 | 24:4964946 | 98992 | 24:4997353 | 99007 | 24:5345133 | 99010 | 24:5453007 | 99011 | 24:5453574 |
| 99019 | 24:5665620 | 99110 | 24:8068578 | 99117 | 24:8174902 | 99126 | 24:8403392 | 99156 | 24:9223598 |
| 99164 | 24:9437183 | 99183 | 24:9782804 | 99185 | 24:9813237 | 99187 | 24:9859970 | 99197 | 24:10080656 |
| 99198 | 24:10082192 | 99199 | 24:10083376 | 99200 | 24:10084233 | 99201 | 24:10085368 | 99202 | 24:10085413 |
| 99203 | 24:10085426 | 99204 | 24:10085505 | 99205 | 24:10085865 | 99206 | 24:10086080 | 99207 | 24:10086129 |
| 99208 | 24:10086191 | 99209 | 24:10087002 | 99210 | 24:10087384 | 99211 | 24:10087677 | 99212 | 24:10088340 |
| 99213 | 24:10089300 | 99214 | 24:10090958 | 99215 | 24:10092604 | 99216 | 24:10092904 | 99217 | 24:10093339 |
| 99218 | 24:10093371 | 99219 | 24:10093377 | 99220 | 24:10093477 | 99221 | 24:10093552 | 99222 | 24:10093626 |
| 99223 | 24:10099234 | 99224 | 24:10100942 | 99225 | 24:10101971 | 99226 | 24:10102022 | 99227 | 24:10102315 |
| 99228 | 24:10102992 | 99229 | 24:10104790 | 99230 | 24:10106712 | 99231 | 24:10112256 | 99232 | 24:10120280 |
| 99233 | 24:10120318 | 99234 | 24:10125493 | 99235 | 24:10127888 | 99236 | 24:10128409 | 99237 | 24:10128428 |
| 99238 | 24:10128467 | 99239 | 24:10129710 | 99240 | 24:10129815 | 99241 | 24:10131022 | 99242 | 24:10131061 |
| 99243 | 24:10131076 | 99244 | 24:10131213 | 99245 | 24:10131371 | 99246 | 24:10131470 | 99247 | 24:10131562 |
| 99248 | 24:10132898 | 99249 | 24:10132907 | 99250 | 24:10134379 | 99251 | 24:10134487 | 99252 | 24:10134521 |
| 99253 | 24:10134705 | 99254 | 24:10134719 | 99255 | 24:10134821 | 99256 | 24:10134859 | 99257 | 24:10134952 |
| 99258 | 24:10134953 | 99259 | 24:10136095 | 99260 | 24:10137902 | 99261 | 24:10137983 | 99262 | 24:10139078 |
| 99263 | 24:10139760 | 99264 | 24:10140006 | 99265 | 24:10140938 | 99266 | 24:10143195 | 99267 | 24:10143925 |
| 99268 | 24:10144011 | 99269 | 24:10144116 | 99270 | 24:10150408 | 99271 | 24:10151986 | 99272 | 24:10153446 |
| 99273 | 24:10155422 | 99291 | 24:10548737 | 99303 | 24:10838109 | 99313 | 24:11081707 | 99347 | 24:11895230 |
| 99374 | 24:12693269 | 99378 | 24:12740073 | 99397 | 24:13255685 | 99401 | 24:13395143 | 99406 | 24:13474854 |
| 99411 | 24:13599253 | 99431 | 24:14202286 | 99432 | 24:14280523 | 99460 | 24:14990004 | 99467 | 24:15215610 |
| 99471 | 24:15361519 | 99472 | 24:15366916 | 99495 | 24:16259312 | 99498 | 24:16327453 | 99514 | 24:16832720 |
| 99517 | 24:16888113 | 99550 | 24:17884468 | 99553 | 24:17978572 | 99564 | 24:18322902 | 99588 | 24:18993346 |
| 99621 | 24:20085016 | 99710 | 24:21387050 | 99713 | 24:21396719 | 99714 | 24:21407863 | 99721 | 24:21479759 |
| 99823 | 24:24049399 | 99835 | 24:24317905 | 99862 | 24:24997696 | 99899 | 24:25782726 | 99918 | 24:26095475 |
| 99926 | 24:26225994 | 99966 | 24:27495986 | 10000 | 24:28406831 | 10000 | 24:28544002 | 10000 | 24:28549242 |
| 10002 | 24:28829549 | 10002 | 24:29046077 | 10007 | 24:30123139 | 10019 | 24:32865289 | 10024 | 24:34044145 |
| 10025 | 24:34324032 | 10028 | 24:35161423 | 10029 | 24:35363476 | 10031 | 24:35787989 | 10034 | 24:36327209 |
| 10034 | 24:36389875 | 10043 | 24:39349798 | 10047 | 24:40319050 | 10048 | 24:40319355 | 10048 | 24:40320188 |
| 10048 | 24:40320243 | 10048 | 24:40320244 | 10048 | 24:40320423 | 10048 | 24:40320519 | 10048 | 24:40320531 |
| 10048 | 24:40320604 | 10048 | 24:40320642 | 10048 | 24:40320662 | 10049 | 24:40346626 | 10049 | 24:40351905 |
| 10062 | 24:43050450 | 10062 | 24:43264177 | 10064 | 24:43757869 | 10064 | 24:44070040 | 10065 | 24:44312012 |
| 10066 | 24:44454841 | 10067 | 24:44630854 | 10068 | 24:44900221 | 10069 | 24:45024408 | 10073 | 24:45883707 |
| 10074 | 24:45892470 | 10074 | 24:45893302 | 10074 | 24:45900861 | 10074 | 24:45906465 | 10074 | 24:45915865 |
| 10074 | 24:45916571 | 10074 | 24:45917829 | 10075 | 24:45935654 | 10075 | 24:45946887 | 10075 | 24:45961034 |
| 10075 | 24:45965027 | 10075 | 24:45966438 | 10075 | 24:45966683 | 10075 | 24:46019888 | 10076 | 24:46021234 |
| 10076 | 24:46031614 | 10076 | 24:46039025 | 10076 | 24:46039978 | 10076 | 24:46040921 | 10076 | 24:46047288 |
| 10076 | 24:46050396 | 10076 | 24:46052145 | 10079 | 24:46519527 | 10090 | 24:49563537 | 10100 | 24:51861629 |
| 10100 | 24:51877952 | 10102 | 24:52249991 | 10102 | 24:52250875 | 10103 | 24:52380242 | 10103 | 24:52572272 |
| 10103 | 24:52579310 | 10108 | 24:53806202 | 10115 | 24:55068839 | 10121 | 24:56483606 | 10121 | 24:56487277 |
| 10123 | 24:56927299 | 10123 | 24:56931903 | 10124 | 24:56935898 | 10129 | 24:58059868 | 10130 | 24:58143848 |
| 10131 | 24:58393476 | 10132 | 24:58514430 | 10133 | 24:58871566 | 10138 | 24:59956065 | 10138 | 24:59956162 |
| 10141 | 24:60385381 | 10141 | 24:60520182 | 10145 | 24:61136358 | 10147 | 24:61660672 | 10151 | 25:326413 |
| 10155 | 25:1326500 | 10157 | 25:1480308 | 10158 | 25:1751313 | 10162 | 25:2262957 | 10162 | 25:2264339 |
| 10162 | 25:2264421 | 10163 | 25:2264518 | 10163 | 25:2264598 | 10163 | 25:2264608 | 10163 | 25:2264757 |
| 10163 | 25:2264786 | 10163 | 25:2265149 | 10163 | 25:2266070 | 10163 | 25:2266258 | 10163 | 25:2266451 |
| 10163 | 25:2266559 | 10164 | 25:2266588 | 10164 | 25:2266682 | 10164 | 25:2266930 | 10164 | 25:2267017 |
| 10164 | 25:2267496 | 10164 | 25:2267627 | 10164 | 25:2267877 | 10164 | 25:2267895 | 10164 | 25:2267998 |
| 10164 | 25:2267999 | 10165 | 25:2268074 | 10165 | 25:2268874 | 10165 | 25:2269160 | 10165 | 25:2269161 |
| 10165 | 25:2269296 | 10165 | 25:2269538 | 10165 | 25:2269574 | 10165 | 25:2324283 | 10168 | 25:2565490 |
| 10175 | 25:3236023 | 10175 | 25:3277932 | 10177 | 25:3531364 | 10177 | 25:3532276 | 10179 | 25:3846717 |
| 10181 | 25:4251363 | 10181 | 25:4273072 | 10182 | 25:4417956 | 10186 | 25:5178359 | 10186 | 25:5356785 |
| 10187 | 25:5358991 | 10187 | 25:5370164 | 10187 | 25:5487714 | 10188 | 25:5507570 | 10188 | 25:5518043 |
| 10188 | 25:5518349 | 10188 | 25:5518412 | 10188 | 25:5519000 | 10188 | 25:5519033 | 10188 | 25:5519088 |
| 10188 | 25:5519241 | 10188 | 25:5519377 | 10189 | 25:5520929 | 10189 | 25:5520954 | 10189 | 25:5523535 |
| 10189 | 25:5524957 | 10189 | 25:5527480 | 10189 | 25:5527963 | 10189 | 25:5563162 | 10189 | 25:5586659 |
| 10189 | 25:5591911 | 10190 | 25:5595291 | 10191 | 25:5986235 | 10192 | 25:6069855 | 10194 | 25:6361028 |
| 10197 | 25:7238527 | 10197 | 25:7238686 | 10197 | 25:7239004 | 10203 | 25:8519593 | 10203 | 25:8697220 |
| 10203 | 25:8699100 | 10204 | 25:8709323 | 10204 | 25:8764411 | 10204 | 25:8773984 | 10205 | 25:8963960 |
| 10206 | 25:9320778 | 10206 | 25:9426676 | 10209 | 25:10079477 | 10210 | 25:10133416 | 10213 | 25:10823773 |
| 10213 | 25:10973032 | 10215 | 25:11195704 | 10215 | 25:11212796 | 10219 | 25:12044897 | 10222 | 25:12552168 |
| 10223 | 25:12921926 | 10223 | 25:12926787 | 10224 | 25:13336640 | 10225 | 25:13422767 | 10226 | 25:13560729 |
| 10226 | 25:13702632 | 10227 | 25:14050888 | 10230 | 25:14538936 | 10231 | 25:14817768 | 10231 | 25:14858436 |
| 10232 | 25:14961033 | 10233 | 25:15333545 | 10236 | 25:15932671 | 10241 | 25:16848884 | 10243 | 25:17233999 |
| 10243 | 25:17237242 | 10245 | 25:17765386 | 10258 | 25:21144052 | 10258 | 25:21184609 | 10261 | 25:21797730 |
| 10262 | 25:22007781 | 10263 | 25:22296079 | 10263 | 25:22329885 | 10265 | 25:22669357 | 10266 | 25:22938890 |
| 10268 | 25:23411155 | 10268 | 25:23449496 | 10268 | 25:23538270 | 10270 | 25:23977366 | 10270 | 25:23979613 |
| 10271 | 25:24161354 | 10272 | 25:24271708 | 10274 | 25:24641425 | 10274 | 25:24667454 | 10281 | 25:25961592 |
| 10281 | 25:26009553 | 10283 | 25:26491942 | 10285 | 25:26753501 | 10287 | 25:27390789 | 10292 | 25:27781936 |
| 10299 | 25:29636939 | 10300 | 25:29838430 | 10300 | 25:29838448 | 10301 | 25:29839578 | 10301 | 25:29844263 |
| 10302 | 25:30146443 | 10306 | 25:31190020 | 10307 | 25:31229333 | 10307 | 25:31297977 | 10309 | 25:31607175 |
| 10313 | 25:32422851 | 10313 | 25:32423232 | 10314 | 25:32564396 | 10317 | 25:33134898 | 10318 | 25:33336358 |
| 10320 | 25:33763001 | 10321 | 25:34018085 | 10323 | 25:34358250 | 10326 | 25:34863162 | 10327 | 25:34993980 |
| 10331 | 25:35712925 | 10333 | 25:35998185 | 10334 | 25:36199960 | 10338 | 25:36479714 | 10345 | 25:37601488 |
| 10354 | 25:39461505 | 10355 | 25:39500543 | 10356 | 25:39824171 | 10356 | 25:39824495 | 10358 | 25:40196620 |
| 10361 | 25:40615618 | 10367 | 25:41358682 | 10373 | 26:1198222 | 10373 | 26:1198334 | 10373 | 26:1252329 |
| 10375 | 26:1854681 | 10376 | 26:2064954 | 10377 | 26:2329446 | 10377 | 26:2473264 | 10378 | 26:2489945 |
| 10378 | 26:2571028 | 10381 | 26:3140966 | 10383 | 26:3713174 | 10384 | 26:3717033 | 10389 | 26:4954983 |
| 10389 | 26:5073658 | 10390 | 26:5545287 | 10395 | 26:7011510 | 10395 | 26:7022453 | 10398 | 26:7885953 |
| 10399 | 26:8061770 | 10399 | 26:8088819 | 10399 | 26:8089138 | 10401 | 26:8426892 | 10401 | 26:8486858 |
| 10402 | 26:8674404 | 10404 | 26:9248331 | 10409 | 26:10405639 | 10410 | 26:10640932 | 10413 | 26:11551549 |
| 10418 | 26:12736512 | 10418 | 26:12824626 | 10418 | 26:12837527 | 10419 | 26:12927334 | 10420 | 26:13161640 |
| 10420 | 26:13230527 | 10421 | 26:13230553 | 10421 | 26:13230674 | 10421 | 26:13230708 | 10421 | 26:13230970 |
| 10422 | 26:13354058 | 10424 | 26:14098514 | 10428 | 26:15245070 | 10433 | 26:16547356 | 10433 | 26:16723176 |
| 10437 | 26:17376800 | 10439 | 26:17759010 | 10442 | 26:18446389 | 10444 | 26:18949002 | 10447 | 26:19421103 |
| 10448 | 26:19560868 | 10451 | 26:20617024 | 10451 | 26:20829028 | 10454 | 26:21273598 | 10454 | 26:21380205 |
| 10460 | 26:23188643 | 10463 | 26:24162535 | 10465 | 26:24594195 | 10470 | 26:25362443 | 10474 | 26:25786538 |
| 10476 | 26:26139139 | 10478 | 26:26718349 | 10478 | 26:26723790 | 10479 | 26:26900204 | 10479 | 26:26928555 |
| 10480 | 26:27118119 | 10480 | 26:27128850 | 10480 | 26:27130646 | 10483 | 26:27876991 | 10484 | 26:28054644 |
| 10484 | 26:28086960 | 10488 | 26:29151186 | 10489 | 26:29673193 | 10490 | 26:29804085 | 10496 | 26:31379196 |
| 10503 | 26:33062542 | 10509 | 26:34211364 | 10509 | 26:34259597 | 10510 | 26:34593375 | 10513 | 26:35102465 |
| 10514 | 26:35442752 | 10516 | 26:35826945 | 10518 | 26:36410448 | 10519 | 26:36629017 | 10524 | 26:38084778 |
| 10525 | 26:38264488 | 10525 | 26:38330297 | 10533 | 26:40288755 | 10541 | 26:42332291 | 10543 | 26:42682604 |
| 10545 | 26:43279886 | 10549 | 26:44209268 | 10554 | 26:45319249 | 10556 | 26:45612666 | 10557 | 26:45867614 |
| 10560 | 26:46499606 | 10562 | 26:46841840 | 10566 | 26:47841265 | 10567 | 26:47878623 | 10567 | 26:48135137 |
| 10568 | 26:48274013 | 10571 | 26:48791917 | 10572 | 26:49108450 | 10575 | 26:49581277 | 10577 | 26:50021533 |
| 10577 | 26:50046769 | 10577 | 26:50172060 | 10578 | 26:50483114 | 10581 | 26:50882810 | 10581 | 26:51112488 |
| 10583 | 26:51406962 | 10583 | 26:51407053 | 10583 | 26:51487956 | 10583 | 26:51520196 | 10591 | 27:1670150 |
| 10595 | 27:2495166 | 10601 | 27:4056109 | 10601 | 27:4058537 | 10602 | 27:4060363 | 10602 | 27:4060531 |
| 10602 | 27:4060533 | 10608 | 27:5431758 | 10616 | 27:7970865 | 10620 | 27:8914781 | 10621 | 27:8978523 |
| 10626 | 27:9933403 | 10627 | 27:9948916 | 10632 | 27:11708333 | 10633 | 27:11961138 | 10634 | 27:12169807 |
| 10636 | 27:12847147 | 10638 | 27:13313622 | 10639 | 27:13498868 | 10639 | 27:13539506 | 10640 | 27:13604776 |
| 10640 | 27:13623320 | 10640 | 27:13722354 | 10641 | 27:13774707 | 10641 | 27:13894721 | 10642 | 27:13894736 |
| 10642 | 27:13895111 | 10642 | 27:13895260 | 10642 | 27:13895824 | 10642 | 27:13895841 | 10642 | 27:13896100 |
| 10642 | 27:13896107 | 10642 | 27:13896340 | 10642 | 27:13896382 | 10642 | 27:13896475 | 10643 | 27:13896495 |
| 10643 | 27:13896544 | 10643 | 27:13896557 | 10643 | 27:13896621 | 10643 | 27:13896676 | 10643 | 27:13897078 |
| 10643 | 27:13897184 | 10643 | 27:13897225 | 10643 | 27:13898435 | 10643 | 27:13898864 | 10644 | 27:13899961 |
| 10644 | 27:13900283 | 10644 | 27:13900709 | 10644 | 27:13900927 | 10644 | 27:13900986 | 10644 | 27:13902739 |
| 10644 | 27:13903517 | 10644 | 27:13903525 | 10644 | 27:13903671 | 10644 | 27:13904229 | 10645 | 27:13904269 |
| 10647 | 27:14467858 | 10648 | 27:14760458 | 10648 | 27:14801389 | 10649 | 27:15058579 | 10651 | 27:15382547 |
| 10654 | 27:15918980 | 10654 | 27:16000987 | 10655 | 27:16074052 | 10658 | 27:16407816 | 10658 | 27:16464527 |
| 10662 | 27:17348090 | 10663 | 27:17555135 | 10669 | 27:19049000 | 10669 | 27:19103942 | 10670 | 27:19175688 |
| 10670 | 27:19178944 | 10671 | 27:19583156 | 10677 | 27:20734233 | 10681 | 27:21907032 | 10685 | 27:23015129 |
| 10685 | 27:23015519 | 10686 | 27:23162886 | 10691 | 27:24155175 | 10694 | 27:24823413 | 10695 | 27:24889870 |
| 10695 | 27:25111470 | 10698 | 27:25597037 | 10698 | 27:25632047 | 10701 | 27:26458109 | 10702 | 27:26491670 |
| 10705 | 27:27075217 | 10705 | 27:27089901 | 10708 | 27:27645675 | 10710 | 27:28160084 | 10711 | 27:28613312 |
| 10712 | 27:28845373 | 10713 | 27:29137179 | 10714 | 27:29174979 | 10717 | 27:29722415 | 10719 | 27:30336321 |
| 10719 | 27:30352172 | 10721 | 27:30751847 | 10721 | 27:30799722 | 10725 | 27:31405475 | 10726 | 27:31691203 |
| 10727 | 27:31936569 | 10727 | 27:31937059 | 10727 | 27:31937078 | 10728 | 27:31938645 | 10728 | 27:31939482 |
| 10728 | 27:31940571 | 10733 | 27:32941799 | 10733 | 27:32957490 | 10734 | 27:33063853 | 10738 | 27:34082682 |
| 10742 | 27:35043316 | 10746 | 27:35480823 | 10746 | 27:35485331 | 10746 | 27:35488554 | 10746 | 27:35489257 |
| 10746 | 27:35506982 | 10747 | 27:35556513 | 10747 | 27:35570251 | 10747 | 27:35709604 | 10748 | 27:35789498 |
| 10748 | 27:35863653 | 10749 | 27:35889925 | 10750 | 27:36291522 | 10751 | 27:36452785 | 10752 | 27:36735236 |
| 10755 | 27:37288845 | 10755 | 27:37353731 | 10756 | 27:37379099 | 10760 | 27:38144204 | 10763 | 27:38793276 |
| 10763 | 27:38844060 | 10766 | 27:39447790 | 10768 | 27:40218010 | 10778 | 27:42642792 | 10779 | 27:43019477 |
| 10780 | 27:43086918 | 10780 | 27:43182706 | 10781 | 27:43500535 | 10782 | 27:43689399 | 10785 | 27:44157299 |
| 10786 | 27:44229484 | 10786 | 27:44268404 | 10790 | 27:45312149 | 10794 | 28:1157605 | 10796 | 28:1342853 |
| 10799 | 28:2055151 | 10800 | 28:2304836 | 10802 | 28:3006891 | 10802 | 28:3047863 | 10803 | 28:3081340 |
| 10803 | 28:3085107 | 10807 | 28:5126492 | 10808 | 28:5287434 | 10809 | 28:5566930 | 10812 | 28:5966745 |
| 10819 | 28:7882083 | 10825 | 28:9096925 | 10827 | 28:9589074 | 10835 | 28:12204786 | 10835 | 28:12216204 |
| 10836 | 28:12230473 | 10836 | 28:12253261 | 10836 | 28:12259647 | 10836 | 28:12260824 | 10836 | 28:12274548 |
| 10836 | 28:12276973 | 10836 | 28:12280112 | 10836 | 28:12281177 | 10837 | 28:12285449 | 10837 | 28:12340669 |
| 10838 | 28:12499169 | 10839 | 28:12767375 | 10842 | 28:13530571 | 10842 | 28:13632810 | 10843 | 28:14061176 |
| 10845 | 28:14464098 | 10846 | 28:14523784 | 10853 | 28:16147418 | 10858 | 28:17255203 | 10858 | 28:17266697 |
| 10859 | 28:17297973 | 10859 | 28:17348454 | 10859 | 28:17452718 | 10860 | 28:17453580 | 10860 | 28:17454068 |
| 10860 | 28:17454138 | 10860 | 28:17454593 | 10860 | 28:17454620 | 10860 | 28:17536228 | 10861 | 28:17642513 |
| 10861 | 28:17917993 | 10867 | 28:19218143 | 10870 | 28:20070339 | 10872 | 28:20595474 | 10877 | 28:22093298 |
| 10882 | 28:23296337 | 10884 | 28:23859369 | 10885 | 28:24072970 | 10886 | 28:24222637 | 10893 | 28:25714994 |
| 10898 | 28:26523238 | 10901 | 28:27080357 | 10903 | 28:27474956 | 10904 | 28:27902972 | 10904 | 28:27904071 |
| 10907 | 28:28055097 | 10908 | 28:28119648 | 10911 | 28:28723390 | 10912 | 28:28968643 | 10914 | 28:29407005 |
| 10919 | 28:30842659 | 10920 | 28:31044910 | 10922 | 28:31459804 | 10924 | 28:31905889 | 10925 | 28:32096800 |
| 10927 | 28:32495037 | 10928 | 28:32698642 | 10929 | 28:32777537 | 10929 | 28:32777974 | 10929 | 28:32780910 |
| 10929 | 28:32792000 | 10929 | 28:32793533 | 10929 | 28:32795812 | 10929 | 28:32796515 | 10929 | 28:32801458 |
| 10930 | 28:32803767 | 10930 | 28:32808502 | 10936 | 28:34084210 | 10941 | 28:34925017 | 10941 | 28:34933980 |
| 10943 | 28:35473082 | 10945 | 28:35713729 | 10946 | 28:36047723 | 10946 | 28:36049682 | 10946 | 28:36059895 |
| 10949 | 28:36735400 | 10952 | 28:37458115 | 10952 | 28:37571711 | 10954 | 28:37918674 | 10955 | 28:38086633 |
| 10956 | 28:38273052 | 10956 | 28:38338458 | 10956 | 28:38411536 | 10958 | 28:38715969 | 10959 | 28:39018331 |
| 10962 | 28:39556352 | 10965 | 28:40413981 | 10973 | 28:40820229 | 10973 | 28:40945850 | 10977 | 28:41644978 |
| 10984 | 28:42940481 | 10986 | 28:43165757 | 10996 | 28:45127262 | 10996 | 28:45146124 | 10997 | 28:45229830 |
| 11003 | 29:1136794 | 11004 | 29:1245711 | 11004 | 29:1296904 | 11007 | 29:1876223 | 11009 | 29:2287359 |
| 11009 | 29:2305683 | 11009 | 29:2369666 | 11010 | 29:2449190 | 11010 | 29:2457832 | 11010 | 29:2458133 |
| 11010 | 29:2466616 | 11011 | 29:2475067 | 11011 | 29:2485044 | 11011 | 29:2491210 | 11012 | 29:2791675 |
| 11012 | 29:2792329 | 11013 | 29:2922954 | 11014 | 29:3034446 | 11014 | 29:3054628 | 11015 | 29:3415853 |
| 11016 | 29:3672128 | 11018 | 29:4149425 | 11021 | 29:4771466 | 11021 | 29:4838513 | 11021 | 29:4985399 |
| 11026 | 29:6292926 | 11027 | 29:6759181 | 11030 | 29:7614767 | 11031 | 29:7747910 | 11032 | 29:8183109 |
| 11033 | 29:8211095 | 11034 | 29:8657356 | 11034 | 29:8692066 | 11041 | 29:10237310 | 11042 | 29:10361704 |
| 11043 | 29:10770679 | 11045 | 29:11203337 | 11049 | 29:11981461 | 11053 | 29:12780503 | 11054 | 29:13287093 |
| 11056 | 29:13704499 | 11056 | 29:13711224 | 11058 | 29:14222266 | 11060 | 29:14863923 | 11061 | 29:15023531 |
| 11061 | 29:15136113 | 11063 | 29:15491770 | 11063 | 29:15518673 | 11063 | 29:15577523 | 11063 | 29:15628215 |
| 11063 | 29:15685760 | 11064 | 29:15764983 | 11065 | 29:16007380 | 11065 | 29:16061265 | 11074 | 29:19039096 |
| 11076 | 29:19634491 | 11078 | 29:20300393 | 11079 | 29:20361512 | 11079 | 29:20457751 | 11080 | 29:20716777 |
| 11081 | 29:20812973 | 11081 | 29:20825149 | 11081 | 29:20965169 | 11082 | 29:21317409 | 11082 | 29:21320200 |
| 11082 | 29:21330819 | 11088 | 29:22764191 | 11089 | 29:23102280 | 11092 | 29:23595059 | 11097 | 29:24932130 |
| 11097 | 29:24968087 | 11106 | 29:27477867 | 11107 | 29:27723748 | 11109 | 29:28108791 | 111110 | 29:28257705 |
| 11118 | 29:29705618 | 11124 | 29:30967685 | 11125 | 29:31273255 | 11126 | 29:31338655 | 11136 | 29:33741610 |
| 11136 | 29:33751577 | 11137 | 29:33898078 | 11139 | 29:34412457 | 11140 | 29:34469913 | 11140 | 29:34497844 |
| 11143 | 29:35142257 | 11143 | 29:35191995 | 11147 | 29:35837500 | 11147 | 29:35923286 | 11148 | 29:36087955 |
| 11148 | 29:36106402 | 11150 | 29:36396078 | 11153 | 29:37001499 | 11153 | 29:37010699 | 11155 | 29:37383025 |
| 11156 | 29:37754439 | 11157 | 29:37797892 | 11159 | 29:39272372 | 11160 | 29:39663800 | 11164 | 29:40480123 |
| 11167 | 29:40880222 | 11168 | 29:41078094 | 11169 | 29:41274073 | 11170 | 29:41379850 | 11170 | 29:41422028 |
| 11170 | 29:41439204 | 11170 | 29:41511246 | 11170 | 29:41535408 | 11175 | 29:42380020 | 11180 | 29:43370086 |
| 11185 | 29:44022611 | 11185 | 29:44090714 | 11185 | 29:44103946 | 11185 | 29:44111412 | 11188 | 29:44504806 |
| 11193 | 29:45854845 | 11193 | 29:45910725 | 11197 | 29:46622198 | 11200 | 29:47401443 | 11202 | 29:47889180 |
| 11203 | 29:47969493 | 11206 | 29:48682297 | 11207 | 29:48937511 | 11207 | 29:48937944 | 11207 | 29:48939020 |
| 11207 | 29:49019691 | 11207 | 29:49027895 | 11208 | 29:49027958 | 11208 | 29:49030254 | 11208 | 29:49031741 |
| 11208 | 29:49031778 | 11208 | 29:49032495 | 11208 | 29:49032522 | 11208 | 29:49034373 | 11208 | 29:49034762 |
| 11208 | 29:49036958 | 11208 | 29:49042618 | 11209 | 29:49047332 | 11209 | 29:49047368 | 11209 | 29:49100292 |
| 11209 | 29:49200650 | 11209 | 29:49201144 | 11214 | 29:50487594 | 11215 | 29:50487813 | 11215 | 29:50490270 |
| 11215 | 29:50531220 | | | | | | | | ; |

and
the sites of the second marker panel are shown in the following table:

| NO. | Position | NO. | Position | NO. | Position | NO. | Position | NO. | Position |
|---|---|---|---|---|---|---|---|---|---|
| 23 | 1: 1010056 | 74 | 1: 1706307 | 78 | 1: 1817641 | 79 | 1: 1821203 | 81 | 1: 1836176 |
| 84 | 1: 1886288 | 87 | 1: 1979359 | 88 | 1: 1997766 | 91 | 1: 2032673 | 109 | 1: 2304587 |
| 112 | 1: 2390062 | 117 | 1: 2749113 | 125 | 1: 3018314 | 138 | 1: 3210484 | 143 | 1: 3282090 |
| 153 | 1: 3479131 | 158 | 1: 3520298 | 174 | 1: 3713849 | 175 | 1: 3716490 | 177 | 1: 3718949 |
| 178 | 1: 3720622 | 179 | 1: 3723615 | 192 | 1: 4021054 | 194 | 1: 4058825 | 197 | 1: 4081869 |
| 200 | 1: 4127064 | 201 | 1: 4130636 | 221 | 1: 4542229 | 245 | 1: 5107763 | 252 | 1: 5359288 |
| 274 | 1: 5770588 | 312 | 1: 6612693 | 320 | 1: 6739866 | 328 | 1: 6816263 | 329 | 1: 6827691 |
| 331 | 1: 6856676 | 333 | 1: 6963461 | 335 | 1: 7027764 | 342 | 1: 7064962 | 349 | 1: 7173547 |
| 351 | 1: 7269335 | 356 | 1: 7378233 | 362 | 1: 7438240 | 363 | 1: 7441167 | 368 | 1: 7637339 |
| 391 | 1: 8312182 | 395 | 1: 8473407 | 406 | 1: 8738949 | 420 | 1: 9069741 | 422 | 1: 9092818 |
| 426 | 1: 9259731 | 431 | 1: 9403159 | 432 | 1: 9442145 | 433 | 1: 9442252 | 434 | 1: 9442256 |
| 435 | 1: 9442802 | 436 | 1: 9467212 | 437 | 1: 9467322 | 439 | 1: 9505564 | 441 | 1: 9525931 |
| 449 | 1: 9675561 | 450 | 1: 9679977 | 451 | 1: 9699218 | 456 | 1: 9843069 | 460 | 1: 9929401 |
| 468 | 1: 10048554 | 472 | 1: 10225421 | 473 | 1: 10225456 | 474 | 1: 10231200 | 476 | 1: 10264906 |
| 477 | 1: 10308984 | 479 | 1: 10360268 | 487 | 1: 10501157 | 490 | 1: 10523191 | 491 | 1: 10537240 |
| 494 | 1: 10563513 | 495 | 1: 10565732 | 498 | 1: 10605121 | 502 | 1: 10771543 | 503 | 1: 10778102 |
| 505 | 1: 10809430 | 506 | 1: 10823521 | 520 | 1: 11223644 | 531 | 1: 11472343 | 535 | 1: 11550314 |
| 544 | 1: 12010431 | 555 | 1: 12378491 | 556 | 1: 12378514 | 566 | 1: 12644235 | 572 | 1: 12803499 |
| 573 | 1: 12803538 | 574 | 1: 12803609 | 584 | 1: 13280975 | 585 | 1: 13288892 | 587 | 1: 13292022 |
| 588 | 1: 13297157 | 591 | 1: 13398597 | 618 | 1: 13725800 | 645 | 1: 14655035 | 648 | 1: 14720755 |
| 668 | 1: 15201042 | 669 | 1: 15201103 | 670 | 1: 15201137 | 695 | 1: 15982024 | 702 | 1: 16155554 |
| 703 | 1: 16155577 | 704 | 1: 16155627 | 705 | 1: 16155668 | 714 | 1: 16447334 | 723 | 1: 16654742 |
| 741 | 1: 17350471 | 748 | 1: 17481344 | 749 | 1: 17481363 | 753 | 1: 17580720 | 774 | 1: 18459894 |
| 799 | 1: 19402363 | 800 | 1: 19409456 | 804 | 1: 19493132 | 814 | 1: 19676162 | 819 | 1: 19929109 |
| 833 | 1: 20279571 | 837 | 1: 20407748 | 840 | 1: 20480157 | 845 | 1: 20570353 | 847 | 1: 20621543 |
| 857 | 1: 20823430 | 860 | 1: 20921794 | 861 | 1: 20974759 | 863 | 1: 21005976 | 868 | 1: 21167645 |
| 872 | 1: 21346196 | 883 | 1: 21646634 | 890 | 1: 21783397 | 896 | 1: 21921230 | 902 | 1: 22076454 |
| 925 | 1: 22688749 | 933 | 1: 22949333 | 947 | 1: 23444110 | 952 | 1: 23709011 | 953 | 1: 23709064 |
| 990 | 1: 24753266 | 993 | 1: 24782983 | 1010 | 1: 25234761 | 1011 | 1: 25234832 | 1023 | 1: 25497647 |
| 1056 | 1: 26533932 | 1058 | 1: 26552621 | 1061 | 1: 26559576 | 1063 | 1: 26611592 | 1067 | 1: 26755277 |
| 1070 | 1: 26880560 | 1072 | 1: 26882959 | 1077 | 1: 26969554 | 1082 | 1: 27132369 | 1084 | 1: 27171038 |
| 1092 | 1: 27401121 | 1134 | 1: 29068209 | 1144 | 1: 29340605 | 1147 | 1: 29403350 | 1151 | 1: 29458506 |
| 1158 | 1: 29712048 | 1165 | 1: 29884320 | 1170 | 1: 29949770 | 1180 | 1: 30194782 | 1201 | 1: 30897798 |
| 1219 | 1: 31546632 | 1227 | 1: 31736868 | 1249 | 1: 32659120 | 1262 | 1: 33136711 | 1265 | 1: 33292309 |
| 1270 | 1: 33496557 | 1272 | 1: 33533012 | 1289 | 1: 33884832 | 1300 | 1: 34174940 | 1309 | 1: 34602877 |
| 1311 | 1: 34663000 | 1313 | 1: 34664612 | 1317 | 1: 34824821 | 1318 | 1: 34824930 | 1321 | 1: 34924925 |
| 1324 | 1: 35000632 | 1331 | 1: 35082343 | 1344 | 1: 35345367 | 1349 | 1: 35407003 | 1362 | 1: 35733756 |
| 1370 | 1: 36018590 | 1372 | 1: 36111561 | 1379 | 1: 36303126 | 1389 | 1: 36436645 | 1392 | 1: 36459421 |
| 1393 | 1: 36459510 | 1402 | 1: 36697707 | 1408 | 1: 36828697 | 1414 | 1: 37032363 | 1415 | 1: 37032440 |
| 1426 | 1: 37411283 | 1428 | 1: 37442242 | 1434 | 1: 37638872 | 1438 | 1: 37686047 | 1440 | 1: 37725772 |
| 1450 | 1: 37956983 | 1453 | 1: 38013891 | 1468 | 1: 38540153 | 1469 | 1: 38599466 | 1476 | 1: 38720880 |
| 1494 | 1: 39249574 | 1505 | 1: 39697516 | 1534 | 1: 40506922 | 1538 | 1: 40629765 | 1548 | 1: 40789010 |
| 1573 | 1: 41686185 | 1579 | 1: 41784113 | 1580 | 1: 41788052 | 1581 | 1: 41795018 | 1582 | 1: 41795589 |
| 1583 | 1: 41796750 | 1584 | 1: 41797989 | 1586 | 1: 41805753 | 1587 | 1: 41835927 | 1588 | 1: 41836033 |
| 1590 | 1: 41886688 | 1600 | 1: 42107409 | 1601 | 1: 42131473 | 1604 | 1: 42159211 | 1605 | 1: 42170788 |
| 1609 | 1: 42224416 | 1611 | 1: 42265122 | 1612 | 1: 42285326 | 1613 | 1: 42293500 | 1619 | 1: 42519517 |
| 1633 | 1: 42917734 | 1640 | 1: 43141355 | 1643 | 1: 43211702 | 1645 | 1: 43225435 | 1647 | 1: 43292695 |
| 1652 | 1: 43443436 | 1656 | 1: 43496586 | 1668 | 1: 43756861 | 1670 | 1: 43772333 | 1672 | 1: 43787631 |
| 1684 | 1: 44030562 | 1686 | 1: 44124010 | 1688 | 1: 44137020 | 1690 | 1: 44236960 | 1700 | 1: 44393495 |
| 1701 | 1: 44422846 | 1708 | 1: 44623944 | 1712 | 1: 44766328 | 1714 | 1: 44820415 | 1715 | 1: 44820480 |
| 1716 | 1: 44820576 | 1717 | 1: 44847495 | 1732 | 1: 44947997 | 1733 | 1: 44956505 | 1734 | 1: 44959367 |
| 1735 | 1: 44960388 | 1737 | 1: 44995524 | 1738 | 1: 44998217 | 1740 | 1: 45030214 | 1741 | 1: 45036157 |
| 1744 | 1: 45061133 | 1748 | 1: 45196717 | 1750 | 1: 45216991 | 1759 | 1: 45365527 | 1760 | 1: 45372759 |
| 1784 | 1: 46185873 | 1793 | 1: 46488716 | 1821 | 1: 47439819 | 1822 | 1: 47457097 | 1823 | 1: 47465147 |
| 1829 | 1: 47631393 | 1852 | 1: 48538887 | 1853 | 1: 48538897 | 1866 | 1: 48823158 | 1893 | 1: 49844520 |
| 1907 | 1: 50327235 | 1913 | 1: 50531202 | 1916 | 1: 50627944 | 1917 | 1: 50631061 | 1918 | 1: 50631100 |
| 1919 | 1: 50637655 | 1924 | 1: 50846942 | 1925 | 1: 50856429 | 1926 | 1: 50874802 | 1928 | 1: 50934316 |
| 1931 | 1: 50978540 | 1934 | 1: 51048823 | 1945 | 1: 51501180 | 1958 | 1: 51956854 | 1960 | 1: 52001408 |
| 1974 | 1: 52418784 | 1975 | 1: 52420721 | 1977 | 1: 52468623 | 1979 | 1: 52499614 | 1980 | 1: 52499623 |
| 1982 | 1: 52592314 | 1983 | 1: 52616071 | 1984 | 1: 52639898 | 1989 | 1: 52845116 | 1999 | 1: 53149622 |
| 2008 | 1: 53293355 | 2011 | 1: 53338531 | 2015 | 1: 53534215 | 2034 | 1: 53685551 | 2036 | 1: 53685641 |
| 2037 | 1: 53685662 | 2038 | 1: 53685710 | 2043 | 1: 53760822 | 2049 | 1: 53972219 | 2055 | 1: 54092473 |
| 2056 | 1: 54092637 | 2057 | 1: 54092679 | 2058 | 1: 54093927 | 2059 | 1: 54101343 | 2068 | 1: 54343019 |
| 2076 | 1: 54460688 | 2088 | 1: 54757168 | 2089 | 1: 54766036 | 2096 | 1: 55026386 | 2097 | 1: 55026433 |
| 2108 | 1: 55421744 | 2110 | 1: 55482490 | 2121 | 1: 55927365 | 2122 | 1: 55964487 | 2123 | 1: 55970922 |
| 2132 | 1: 56253478 | 2138 | 1: 56370458 | 2142 | 1: 56472558 | 2145 | 1: 56498727 | 2147 | 1: 56540591 |
| 2154 | 1: 56759097 | 2162 | 1: 56937696 | 2165 | 1: 56975870 | 2170 | 1: 57137491 | 2173 | 1: 57217658 |
| 2174 | 1: 57217807 | 2175 | 1: 57251079 | 2176 | 1: 57266134 | 2178 | 1: 57309255 | 2181 | 1: 57398938 |
| 2184 | 1: 57490618 | 2185 | 1: 57560306 | 2197 | 1: 57870387 | 2198 | 1: 57917830 | 2203 | 1: 58030359 |
| 2205 | 1: 58075432 | 2206 | 1: 58075463 | 2207 | 1: 58075499 | 2208 | 1: 58075521 | 2209 | 1: 58078354 |
| 2210 | 1: 58078466 | 2211 | 1: 58097510 | 2221 | 1: 58305398 | 2222 | 1: 58305455 | 2223 | 1: 58315322 |
| 2224 | 1: 58315443 | 2225 | 1: 58315990 | 2231 | 1: 58540410 | 2232 | 1: 58618667 | 2236 | 1: 58750747 |
| 2237 | 1: 58773333 | 2240 | 1: 58818870 | 2250 | 1: 59034012 | 2252 | 1: 59104762 | 2258 | 1: 59200011 |
| 2259 | 1: 59203569 | 2263 | 1: 59371385 | 2270 | 1: 59656138 | 2279 | 1: 59830679 | 2293 | 1: 60123244 |
| 2318 | 1: 60784872 | 2325 | 1: 60918912 | 2330 | 1: 61097118 | 2333 | 1: 61128757 | 2336 | 1: 61160140 |
| 2369 | 1: 62066946 | 2370 | 1: 62092465 | 2386 | 1: 62457515 | 2399 | 1: 62621737 | 2402 | 1: 62637571 |
| 2406 | 1: 62817788 | 2407 | 1: 62817866 | 2428 | 1: 63139698 | 2438 | 1: 63427104 | 2440 | 1: 63476336 |
| 2441 | 1: 63517812 | 2451 | 1: 63851933 | 2456 | 1: 64008039 | 2461 | 1: 64183671 | 2463 | 1: 64200486 |
| 2464 | 1: 64200539 | 2466 | 1: 64262468 | 2477 | 1: 64431800 | 2478 | 1: 64462597 | 2479 | 1: 64462675 |
| 2480 | 1: 64463152 | 2481 | 1: 64463254 | 2482 | 1: 64467138 | 2483 | 1: 64467211 | 2486 | 1: 64505435 |
| 2496 | 1: 64824564 | 2502 | 1: 64940185 | 2509 | 1: 65157725 | 2512 | 1: 65199200 | 2513 | 1: 65216449 |
| 2514 | 1: 65224381 | 2515 | 1: 65228988 | 2516 | 1: 65239311 | 2520 | 1: 65377133 | 2545 | 1: 66100053 |
| 2547 | 1: 66136711 | 2557 | 1: 66502772 | 2558 | 1: 66511698 | 2559 | 1: 66515809 | 2561 | 1: 66547988 |
| 2569 | 1: 66694865 | 2581 | 1: 67173187 | 2588 | 1: 67320099 | 2590 | 1: 67331681 | 2591 | 1: 67411736 |
| 2593 | 1: 67432038 | 2594 | 1: 67439085 | 2596 | 1: 67473778 | 2604 | 1: 67760033 | 2608 | 1: 67861511 |
| 2618 | 1: 68238481 | 2619 | 1: 68259739 | 2623 | 1: 68332459 | 2627 | 1: 68449381 | 2628 | 1: 68488305 |
| 2630 | 1: 68501205 | 2631 | 1: 68517809 | 2635 | 1: 68649543 | 2637 | 1: 68680831 | 2638 | 1: 68682422 |
| 2639 | 1: 68682461 | 2640 | 1: 68690274 | 2644 | 1: 68784544 | 2646 | 1: 68792494 | 2647 | 1: 68793488 |
| 2648 | 1: 68793540 | 2649 | 1: 68793542 | 2650 | 1: 68793543 | 2651 | 1: 68797693 | 2656 | 1: 68869228 |
| 2663 | 1: 68996534 | 2668 | 1: 69017745 | 2670 | 1: 69042317 | 2671 | 1: 69047274 | 2672 | 1: 69047282 |
| 2673 | 1: 69047320 | 2679 | 1: 69124262 | 2682 | 1: 69203913 | 2684 | 1: 69217319 | 2685 | 1: 69217672 |
| 2698 | 1: 69582986 | 2699 | 1: 69585403 | 2704 | 1: 69764910 | 2712 | 1: 69883156 | 2717 | 1: 69982394 |
| 2720 | 1: 70106968 | 2725 | 1: 70231031 | 2726 | 1: 70231761 | 2727 | 1: 70248171 | 2730 | 1: 70343458 |
| 2732 | 1: 70365636 | 2734 | 1: 70390927 | 2741 | 1: 70466635 | 2742 | 1: 70466719 | 2743 | 1: 70466765 |
| 2744 | 1: 70468358 | 2745 | 1: 70473449 | 2746 | 1: 70473451 | 2747 | 1: 70473452 | 2748 | 1: 70473471 |
| 2749 | 1: 70473493 | 2750 | 1: 70485290 | 2751 | 1: 70485353 | 2753 | 1: 70494055 | 2754 | 1: 70497518 |
| 2755 | 1: 70500089 | 2762 | 1: 70616796 | 2771 | 1: 70783887 | 2772 | 1: 70791872 | 2773 | 1: 70801580 |
| 2790 | 1: 71415748 | 2791 | 1: 71416509 | 2792 | 1: 71416751 | 2793 | 1: 71416881 | 2794 | 1: 71416890 |
| 2803 | 1: 71609698 | 2817 | 1: 72069890 | 2819 | 1: 72081243 | 2820 | 1: 72090597 | 2834 | 1: 72454020 |
| 2836 | 1: 72484009 | 2839 | 1: 72519103 | 2849 | 1: 72679096 | 2850 | 1: 72679109 | 2851 | 1: 72679167 |
| 2854 | 1: 72714931 | 2855 | 1: 72726681 | 2856 | 1: 72732857 | 2859 | 1: 72803314 | 2863 | 1: 72952213 |
| 2864 | 1: 72955756 | 2866 | 1: 73093141 | 2871 | 1: 73187152 | 2872 | 1: 73207902 | 2879 | 1: 73477972 |
| 2883 | 1: 73591733 | 2887 | 1: 73663050 | 2888 | 1: 73671409 | 2890 | 1: 73744599 | 2898 | 1: 73902125 |
| 2902 | 1: 73954764 | 2908 | 1: 74183606 | 2915 | 1: 74460825 | 2917 | 1: 74460955 | 2918 | 1: 74489282 |
| 2919 | 1: 74490359 | 2922 | 1: 74553801 | 2935 | 1: 74976635 | 2957 | 1: 75592224 | 2960 | 1: 75682742 |
| 2967 | 1: 75798251 | 2968 | 1: 75798252 | 2969 | 1: 75798317 | 2972 | 1: 75852855 | 2980 | 1: 76051714 |
| 3013 | 1: 77045567 | 3020 | 1: 77255234 | 3029 | 1: 77397999 | 3032 | 1: 77443583 | 3033 | 1: 77469880 |
| 3036 | 1: 77593114 | 3038 | 1: 77616604 | 3040 | 1: 77652308 | 3044 | 1: 77698757 | 3048 | 1: 77727505 |
| 3054 | 1: 77958907 | 3074 | 1: 78375438 | 3077 | 1: 78445734 | 3082 | 1: 78556250 | 3083 | 1: 78566682 |
| 3093 | 1: 78911959 | 3095 | 1: 78989138 | 3098 | 1: 79082187 | 3101 | 1: 79138960 | 3102 | 1: 79169504 |
| 3105 | 1: 79213373 | 3107 | 1: 79242860 | 3110 | 1: 79298445 | 3114 | 1: 79357690 | 3118 | 1: 79485282 |
| 3123 | 1: 79562994 | 3156 | 1: 80023498 | 3161 | 1: 80069552 | 3163 | 1: 80074105 | 3172 | 1: 80391190 |
| 3173 | 1: 80413261 | 3183 | 1: 80685030 | 3192 | 1: 80940339 | 3196 | 1: 81028408 | 3201 | 1: 81051808 |
| 3208 | 1: 81170302 | 3209 | 1: 81183483 | 3215 | 1: 81348584 | 3229 | 1: 81706937 | 3235 | 1: 81878324 |
| 3240 | 1: 82041541 | 3241 | 1: 82041559 | 3242 | 1: 82047704 | 3246 | 1: 82149539 | 3258 | 1: 82473626 |
| 3264 | 1: 82519837 | 3265 | 1: 82541328 | 3266 | 1: 82541450 | 3268 | 1: 82558245 | 3273 | 1: 82643904 |
| 3276 | 1: 82705721 | 3278 | 1: 82758391 | 3284 | 1: 83070275 | 3285 | 1: 83097205 | 3286 | 1: 83133001 |
| 3291 | 1: 83278833 | 3292 | 1: 83284140 | 3293 | 1: 83330542 | 3306 | 1: 83810343 | 3310 | 1: 83974373 |
| 3312 | 1: 84000147 | 3315 | 1: 84047005 | 3316 | 1: 84058598 | 3338 | 1: 85042289 | 3344 | 1: 85087002 |
| 3346 | 1: 85130568 | 3368 | 1: 85947378 | 3369 | 1: 85948692 | 3371 | 1: 85976228 | 3374 | 1: 86122181 |
| 3375 | 1: 86146073 | 3376 | 1: 86156408 | 3377 | 1: 86160591 | 3378 | 1: 86177464 | 3379 | 1: 86185749 |
| 3380 | 1: 86186427 | 3394 | 1: 86631754 | 3406 | 1: 86967586 | 3420 | 1: 87399098 | 3421 | 1: 87399338 |
| 3422 | 1: 87414899 | 3428 | 1: 87574019 | 3431 | 1: 87648729 | 3434 | 1: 87752223 | 3441 | 1: 88012422 |
| 3467 | 1: 88872312 | 3468 | 1: 88874634 | 3469 | 1: 88880495 | 3471 | 1: 88896168 | 3472 | 1: 88908213 |
| 3474 | 1: 88987015 | 3476 | 1: 89086745 | 3478 | 1: 89097927 | 3479 | 1: 89098024 | 3482 | 1: 89224144 |
| 3484 | 1: 89300003 | 3486 | 1: 89343474 | 3487 | 1: 89349576 | 3488 | 1: 89383250 | 3492 | 1: 89498954 |
| 3494 | 1: 89539395 | 3495 | 1: 89539442 | 3508 | 1: 89796658 | 3509 | 1: 89799413 | 3522 | 1: 90043900 |
| 3524 | 1: 90075379 | 3550 | 1: 90588788 | 3552 | 1: 90615269 | 3554 | 1: 90648889 | 3576 | 1: 91255865 |
| 3577 | 1: 91285295 | 3593 | 1: 91706481 | 3596 | 1: 91737921 | 3597 | 1: 91739554 | 3602 | 1: 91928880 |
| 3609 | 1: 92184004 | 3623 | 1: 92503312 | 3668 | 1: 93883957 | 3670 | 1: 93930496 | 3674 | 1: 94035144 |
| 3675 | 1: 94035267 | 3680 | 1: 94131411 | 3681 | 1: 94134092 | 3699 | 1: 94576910 | 3709 | 1: 94877027 |
| 3729 | 1: 95423137 | 3730 | 1: 95439594 | 3731 | 1: 95455255 | 3733 | 1: 95478530 | 3734 | 1: 95494762 |
| 3745 | 1: 95806271 | 3747 | 1: 95893257 | 3752 | 1: 96002349 | 3754 | 1: 96026129 | 3756 | 1: 96084552 |
| 3757 | 1: 96085021 | 3760 | 1: 96180861 | 3764 | 1: 96218350 | 3765 | 1: 96220152 | 3770 | 1: 96344874 |
| 3779 | 1: 96474332 | 3784 | 1: 96640052 | 3801 | 1: 97152251 | 3819 | 1: 97537514 | 3832 | 1: 97878133 |
| 3843 | 1: 98148099 | 3847 | 1: 98313182 | 3857 | 1: 98673491 | 3867 | 1: 98891260 | 3876 | 1: 99200317 |
| 3882 | 1: 99297524 | 3883 | 1: 99323405 | 3886 | 1: 99418906 | 3890 | 1: 99501232 | 3906 | 1: 99941713 |
| 3914 | 1: 100198782 | 3917 | 1: 100296109 | 3919 | 1: 100321230 | 3927 | 1: 100567937 | 3948 | 1: 101273216 |
| 3969 | 1: 101894473 | 3980 | 1: 102388029 | 3981 | 1: 102388112 | 3988 | 1: 102582187 | 3990 | 1: 102591751 |
| 3993 | 1: 102638499 | 3995 | 1: 102646032 | 3997 | 1: 102698749 | 4004 | 1: 102911722 | 4008 | 1: 103023798 |
| 4037 | 1: 104209003 | 4038 | 1: 104263712 | 4045 | 1: 104522827 | 4053 | 1: 104831357 | 4058 | 1: 104915762 |
| 4060 | 1: 104987991 | 4061 | 1: 105015424 | 4074 | 1: 105367807 | 4079 | 1: 105447199 | 4103 | 1: 106048772 |
| 4108 | 1: 106226124 | 4111 | 1: 106284785 | 4123 | 1: 106497025 | 4144 | 1: 107326886 | 4149 | 1: 107424074 |
| 4153 | 1: 107500490 | 4154 | 1: 107538852 | 4155 | 1: 107544465 | 4156 | 1: 107544536 | 4161 | 1: 107781031 |
| 4164 | 1: 107863018 | 4170 | 1: 108114016 | 4189 | 1: 108515165 | 4191 | 1: 108556182 | 4192 | 1: 108556227 |
| 4203 | 1: 108851719 | 4204 | 1: 108851745 | 4205 | 1: 108853578 | 4206 | 1: 108854832 | 4207 | 1: 108870778 |
| 4208 | 1: 108874776 | 4209 | 1: 108881505 | 4210 | 1: 108884859 | 4211 | 1: 108894708 | 4212 | 1: 108894728 |
| 4213 | 1: 108896221 | 4230 | 1: 109527893 | 4231 | 1: 109527919 | 4245 | 1: 109820651 | 4250 | 1: 109870082 |
| 4266 | 1: 110126279 | 4267 | 1: 110175621 | 4269 | 1: 110196398 | 4273 | 1: 110293441 | 4276 | 1: 110341970 |
| 4277 | 1: 110342054 | 4278 | 1: 110342111 | 4283 | 1: 110476863 | 4288 | 1: 110573226 | 4289 | 1: 110590869 |
| 4292 | 1: 110633655 | 4293 | 1: 110653778 | 4295 | 1: 110665646 | 4296 | 1: 110666740 | 4302 | 1: 110816709 |
| 4309 | 1: 111186619 | 4310 | 1: 111194151 | 4311 | 1: 111199408 | 4312 | 1: 111247418 | 4313 | 1: 111268099 |
| 4325 | 1: 111708777 | 4327 | 1: 111748641 | 4331 | 1: 111775768 | 4332 | 1: 111775805 | 4333 | 1: 111798208 |
| 4345 | 1: 112208772 | 4353 | 1: 112368514 | 4354 | 1: 112398275 | 4355 | 1: 112400081 | 4361 | 1: 112555088 |
| 4362 | 1: 112555094 | 4363 | 1: 112555107 | 4364 | 1: 112555108 | 4365 | 1: 112555300 | 4366 | 1: 112555322 |
| 4367 | 1: 112555340 | 4368 | 1: 112555460 | 4369 | 1: 112555492 | 4370 | 1: 112556677 | 4371 | 1: 112556716 |
| 4372 | 1: 112556776 | 4375 | 1: 112581875 | 4376 | 1: 112581930 | 4377 | 1: 112581975 | 4378 | 1: 112595293 |
| 4400 | 1: 113354543 | 4403 | 1: 113365903 | 4404 | 1: 113369018 | 4405 | 1: 113385595 | 4406 | 1: 113385624 |
| 4407 | 1: 113385658 | 4417 | 1: 113584302 | 4457 | 1: 114467455 | 4460 | 1: 114596976 | 4462 | 1: 114728742 |
| 4465 | 1: 114816068 | 4468 | 1: 114840022 | 4488 | 1: 115324305 | 4490 | 1: 115377104 | 4491 | 1: 115413995 |
| 4496 | 1: 115579666 | 4502 | 1: 115790600 | 4528 | 1: 116754887 | 4532 | 1: 116818074 | 4533 | 1: 116821719 |
| 4535 | 1: 116860895 | 4538 | 1: 116891857 | 4540 | 1: 117003117 | 4546 | 1: 117113278 | 4556 | 1: 117319749 |
| 4563 | 1: 117420923 | 4564 | 1: 117421250 | 4576 | 1: 117738265 | 4578 | 1: 117758868 | 4586 | 1: 117887255 |
| 4587 | 1: 117897860 | 4588 | 1: 117912537 | 4589 | 1: 117935755 | 4591 | 1: 117945817 | 4597 | 1: 118048415 |
| 4598 | 1: 118085158 | 4600 | 1: 118114537 | 4602 | 1: 118133789 | 4605 | 1: 118196612 | 4608 | 1: 118214654 |
| 4609 | 1: 118227302 | 4610 | 1: 118256496 | 4611 | 1: 118257517 | 4612 | 1: 118259606 | 4613 | 1: 118273499 |
| 4614 | 1: 118276261 | 4615 | 1: 118276735 | 4616 | 1: 118277267 | 4617 | 1: 118278387 | 4618 | 1: 118284875 |
| 4619 | 1: 118286166 | 4620 | 1: 118288018 | 4621 | 1: 118290177 | 4622 | 1: 118295131 | 4635 | 1: 118530178 |
| 4636 | 1: 118544527 | 4642 | 1: 118690300 | 4643 | 1: 118690309 | 4652 | 1: 118962324 | 4654 | 1: 118985100 |
| 4665 | 1: 119176913 | 4666 | 1: 119189605 | 4667 | 1: 119210631 | 4672 | 1: 119350100 | 4683 | 1: 119561515 |
| 4684 | 1: 119561537 | 4685 | 1: 119561564 | 4701 | 1: 120064732 | 4712 | 1: 120386879 | 4722 | 1: 120500835 |
| 4726 | 1: 120563845 | 4760 | 1: 121647921 | 4762 | 1: 121707542 | 4771 | 1: 121946641 | 4781 | 1: 122232742 |
| 4785 | 1: 122286938 | 4786 | 1: 122287045 | 4790 | 1: 122500672 | 4794 | 1: 122540031 | 4799 | 1: 122654668 |
| 4801 | 1: 122686036 | 4807 | 1: 122783659 | 4808 | 1: 122784649 | 4810 | 1: 122849903 | 4811 | 1: 122901551 |
| 4814 | 1: 123034294 | 4819 | 1: 123266689 | 4821 | 1: 123270508 | 4827 | 1: 123475726 | 4835 | 1: 123679492 |
| 4836 | 1: 123692405 | 4837 | 1: 123706359 | 4838 | 1: 123709988 | 4839 | 1: 123720581 | 4848 | 1: 124014834 |
| 4854 | 1: 124194081 | 4860 | 1: 124271732 | 4861 | 1: 124271768 | 4862 | 1: 124271786 | 4863 | 1: 124314731 |
| 4864 | 1: 124324829 | 4865 | 1: 124331752 | 4866 | 1: 124343187 | 4870 | 1: 124506907 | 4871 | 1: 124528147 |
| 4878 | 1: 124666898 | 4879 | 1: 124672817 | 4886 | 1: 124887464 | 4891 | 1: 124948606 | 4894 | 1: 124980393 |
| 4895 | 1: 124980562 | 4898 | 1: 125121982 | 4902 | 1: 125269301 | 4904 | 1: 125360929 | 4905 | 1: 125363719 |
| 4907 | 1: 125382225 | 4908 | 1: 125386885 | 4913 | 1: 125521147 | 4919 | 1: 125583210 | 4920 | 1: 125608898 |
| 4922 | 1: 125609041 | 4928 | 1: 125643614 | 4944 | 1: 125851156 | 4946 | 1: 125931085 | 4951 | 1: 126050865 |
| 4953 | 1: 126166588 | 4954 | 1: 126167017 | 4955 | 1: 126188011 | 4962 | 1: 126320901 | 4970 | 1: 126452685 |
| 4979 | 1: 126828386 | 4981 | 1: 126867108 | 4989 | 1: 127247333 | 4997 | 1: 127448104 | 5001 | 1: 127536751 |
| 5004 | 1: 127609705 | 5007 | 1: 127732186 | 5010 | 1: 127791769 | 5011 | 1: 127843966 | 5015 | 1: 127884865 |
| 5020 | 1: 127963540 | 5022 | 1: 128091201 | 5032 | 1: 128267812 | 5037 | 1: 128374395 | 5042 | 1: 128512081 |
| 5073 | 1: 129150388 | 5080 | 1: 129402104 | 5081 | 1: 129418328 | 5089 | 1: 129590492 | 5090 | 1: 129591720 |
| 5091 | 1: 129592529 | 5095 | 1: 129754416 | 5127 | 1: 130632012 | 5128 | 1: 130658736 | 5130 | 1: 130720354 |
| 5131 | 1: 130728433 | 5134 | 1: 130792978 | 5137 | 1: 130920062 | 5140 | 1: 130954555 | 5179 | 1: 131419104 |
| 5222 | 1: 131860577 | 5242 | 1: 132473942 | 5253 | 1: 132876761 | 5264 | 1: 133379843 | 5265 | 1: 133384149 |
| 5266 | 1: 133394080 | 5271 | 1: 133540327 | 5272 | 1: 133591491 | 5289 | 1: 134186323 | 5294 | 1: 134285791 |
| 5298 | 1: 134384145 | 5300 | 1: 134413818 | 5301 | 1: 134413821 | 5306 | 1: 134477738 | 5324 | 1: 134948789 |
| 5325 | 1: 134961346 | 5327 | 1: 134973908 | 5329 | 1: 135015189 | 5331 | 1: 135049866 | 5337 | 1: 135208575 |
| 5341 | 1: 135315701 | 5342 | 1: 135345248 | 5346 | 1: 135410192 | 5347 | 1: 135414049 | 5360 | 1: 135642886 |
| 5361 | 1: 135643955 | 5362 | 1: 135644811 | 5363 | 1: 135653473 | 5366 | 1: 135665914 | 5367 | 1: 135666200 |
| 5368 | 1: 135666237 | 5369 | 1: 135666283 | 5370 | 1: 135670574 | 5371 | 1: 135672004 | 5372 | 1: 135694251 |
| 5374 | 1: 135753894 | 5375 | 1: 135753940 | 5376 | 1: 135753983 | 5377 | 1: 135754044 | 5378 | 1: 135754264 |
| 5379 | 1: 135754300 | 5380 | 1: 135758284 | 5381 | 1: 135758290 | 5382 | 1: 135758332 | 5383 | 1: 135762033 |
| 5384 | 1: 135763704 | 5385 | 1: 135769522 | 5397 | 1: 135880893 | 5399 | 1: 135916803 | 5400 | 1: 135927623 |
| 5401 | 1: 135972509 | 5402 | 1: 135974364 | 5405 | 1: 136033754 | 5406 | 1: 136047835 | 5413 | 1: 136204362 |
| 5415 | 1: 136311162 | 5427 | 1: 136734614 | 5434 | 1: 137049621 | 5438 | 1: 137106364 | 5443 | 1: 137294127 |
| 5472 | 1: 138643152 | 5473 | 1: 138647040 | 5477 | 1: 138728014 | 5478 | 1: 138739021 | 5483 | 1: 138798912 |
| 5489 | 1: 138994500 | 5498 | 1: 139184610 | 5505 | 1: 139411806 | 5508 | 1: 139449113 | 5521 | 1: 139899458 |
| 5536 | 1: 140266541 | 5545 | 1: 140543741 | 5554 | 1: 140754935 | 5555 | 1: 140755072 | 5558 | 1: 140876675 |
| 5575 | 1: 141145402 | 5579 | 1: 141316735 | 5580 | 1: 141319127 | 5581 | 1: 141319994 | 5583 | 1: 141339227 |
| 5584 | 1: 141345591 | 5585 | 1: 141362575 | 5586 | 1: 141364352 | 5587 | 1: 141374294 | 5588 | 1: 141376997 |
| 5590 | 1: 141395382 | 5591 | 1: 141421683 | 5597 | 1: 141511318 | 5601 | 1: 141610105 | 5602 | 1: 141610924 |
| 5604 | 1: 141627871 | 5606 | 1: 141628909 | 5608 | 1: 141649600 | 5610 | 1: 141651362 | 5611 | 1: 141653130 |
| 5612 | 1: 141653134 | 5613 | 1: 141654271 | 5614 | 1: 141654547 | 5615 | 1: 141654551 | 5617 | 1: 141667906 |
| 5622 | 1: 141734030 | 5623 | 1: 141741068 | 5625 | 1: 141823289 | 5633 | 1: 142000532 | 5641 | 1: 142239516 |
| 5643 | 1: 142251697 | 5651 | 1: 142362891 | 5654 | 1: 142393561 | 5657 | 1: 142478032 | 5659 | 1: 142489032 |
| 5667 | 1: 142635123 | 5672 | 1: 142787751 | 5693 | 1: 143128593 | 5700 | 1: 143227118 | 5702 | 1: 143334405 |
| 5706 | 1: 143406113 | 5711 | 1: 143569447 | 5712 | 1: 143579707 | 5713 | 1: 143582830 | 5714 | 1: 143588692 |
| 5715 | 1: 143591196 | 5716 | 1: 143593029 | 5717 | 1: 143594628 | 5726 | 1: 143746215 | 5728 | 1: 143749139 |
| 5729 | 1: 143752954 | 5747 | 1: 144050118 | 5748 | 1: 144050120 | 5757 | 1: 144333701 | 5759 | 1: 144372951 |
| 5763 | 1: 144435905 | 5772 | 1: 144635480 | 5776 | 1: 144757986 | 5780 | 1: 144787067 | 5781 | 1: 144809797 |
| 5786 | 1: 144894365 | 5794 | 1: 145080672 | 5797 | 1: 145128073 | 5799 | 1: 145136789 | 5802 | 1: 145176107 |
| 5804 | 1: 145282436 | 5813 | 1: 145459919 | 5821 | 1: 145623145 | 5822 | 1: 145623795 | 5825 | 1: 145673098 |
| 5826 | 1: 145677709 | 5834 | 1: 145827286 | 5835 | 1: 145830272 | 5837 | 1: 145860541 | 5838 | 1: 145860568 |
| 5839 | 1: 145866834 | 5840 | 1: 145866900 | 5841 | 1: 145873941 | 5842 | 1: 145873979 | 5843 | 1: 145874030 |
| 5848 | 1: 145951072 | 5849 | 1: 145963833 | 5850 | 1: 145968413 | 5852 | 1: 145972525 | 5853 | 1: 145975945 |
| 5854 | 1: 145977236 | 5862 | 1: 146052159 | 5871 | 1: 146351589 | 5874 | 1: 146420270 | 5892 | 1: 146975928 |
| 5894 | 1: 147008926 | 5899 | 1: 147098097 | 5902 | 1: 147122843 | 5918 | 1: 147397092 | 5919 | 1: 147417101 |
| 5921 | 1: 147431978 | 5927 | 1: 147563989 | 5941 | 1: 147846791 | 5946 | 1: 147903408 | 5978 | 1: 148430252 |
| 5979 | 1: 148466864 | 5981 | 1: 148508063 | 5982 | 1: 148508133 | 5984 | 1: 148513206 | 5985 | 1: 148549091 |
| 5994 | 1: 148736098 | 6000 | 1: 148871697 | 6004 | 1: 148964961 | 6010 | 1: 149177892 | 6011 | 1: 149189606 |
| 6014 | 1: 149317573 | 6015 | 1: 149320003 | 6016 | 1: 149323616 | 6017 | 1: 149324955 | 6018 | 1: 149328203 |
| 6019 | 1: 149340128 | 6020 | 1: 149342542 | 6021 | 1: 149348963 | 6022 | 1: 149351545 | 6023 | 1: 149358100 |
| 6024 | 1: 149360084 | 6025 | 1: 149362014 | 6026 | 1: 149366388 | 6027 | 1: 149367120 | 6028 | 1: 149368535 |
| 6029 | 1: 149371783 | 6030 | 1: 149374604 | 6031 | 1: 149375645 | 6032 | 1: 149376574 | 6033 | 1: 149378550 |
| 6034 | 1: 149379934 | 6035 | 1: 149380878 | 6036 | 1: 149381954 | 6037 | 1: 149390100 | 6042 | 1: 149443669 |
| 6049 | 1: 149642436 | 6065 | 1: 149898235 | 6066 | 1: 149902664 | 6068 | 1: 149929034 | 6077 | 1: 150129027 |
| 6078 | 1: 150129035 | 6079 | 1: 150129044 | 6080 | 1: 150129048 | 6081 | 1: 150144069 | 6083 | 1: 150151876 |
| 6093 | 1: 150440522 | 6106 | 1: 150701995 | 6109 | 1: 150741379 | 6119 | 1: 151001062 | 6122 | 1: 151048846 |
| 6124 | 1: 151109660 | 6134 | 1: 151324257 | 6135 | 1: 151326467 | 6148 | 1: 151556745 | 6149 | 1: 151560943 |
| 6150 | 1: 151561380 | 6154 | 1: 151623827 | 6158 | 1: 151643959 | 6161 | 1: 151668710 | 6162 | 1: 151671521 |
| 6163 | 1: 151673956 | 6164 | 1: 151675356 | 6165 | 1: 151677189 | 6182 | 1: 151922660 | 6186 | 1: 152015544 |
| 6189 | 1: 152071824 | 6215 | 1: 152347862 | 6235 | 1: 152417708 | 6237 | 1: 152422584 | 6238 | 1: 152428562 |
| 6249 | 1: 152632298 | 6252 | 1: 152678335 | 6254 | 1: 152745164 | 6260 | 1: 152826141 | 6279 | 1: 153250410 |
| 6301 | 1: 153688732 | 6308 | 1: 153828051 | 6322 | 1: 154004623 | 6323 | 1: 154009064 | 6324 | 1: 154009137 |
| 6325 | 1: 154009145 | 6327 | 1: 154019636 | 6329 | 1: 154038751 | 6341 | 1: 154370113 | 6358 | 1: 154858681 |
| 6388 | 1: 155496781 | 6400 | 1: 155679617 | 6401 | 1: 155679654 | 6402 | 1: 155679691 | 6403 | 1: 155679758 |
| 6404 | 1: 155679762 | 6407 | 1: 155716904 | 6429 | 1: 156040352 | 6434 | 1: 156108198 | 6469 | 1: 156749778 |
| 6502 | 1: 157422591 | 6505 | 1: 157455223 | 6508 | 1: 157589021 | 6514 | 1: 157988428 | 6517 | 1: 158029386 |
| 6518 | 1: 158031759 | 6521 | 1: 158140847 | 6522 | 1: 158227949 | 6550 | 2: 773366 | 6570 | 2: 1116337 |
| 6571 | 2: 1120855 | 6576 | 2: 1174595 | 6579 | 2: 1234786 | 6580 | 2: 1239635 | 6584 | 2: 1280967 |
| 6585 | 2: 1281046 | 6586 | 2: 1286624 | 6587 | 2: 1286642 | 6588 | 2: 1286747 | 6607 | 2: 1669475 |
| 6615 | 2: 1857324 | 6616 | 2: 1859648 | 6623 | 2: 1996597 | 6642 | 2: 2733310 | 6649 | 2: 2936048 |
| 6657 | 2: 3205071 | 6671 | 2: 3644419 | 6679 | 2: 3866542 | 6680 | 2: 3866584 | 6688 | 2: 4033904 |
| 6692 | 2: 4056030 | 6693 | 2: 4056116 | 6702 | 2: 4244343 | 6705 | 2: 4311599 | 6706 | 2: 4311602 |
| 6707 | 2: 4311756 | 6708 | 2: 4318724 | 6710 | 2: 4360783 | 6719 | 2: 4631707 | 6720 | 2: 4634839 |
| 6721 | 2: 4634862 | 6722 | 2: 4634886 | 6723 | 2: 4634902 | 6724 | 2: 4634989 | 6725 | 2: 4655242 |
| 6727 | 2: 4687767 | 6731 | 2: 4782801 | 6749 | 2: 5105483 | 6754 | 2: 5312133 | 6757 | 2: 5343162 |
| 6760 | 2: 5372449 | 6767 | 2: 5418497 | 6770 | 2: 5433956 | 6771 | 2: 5437701 | 6776 | 2: 5564286 |
| 6778 | 2: 5598208 | 6780 | 2: 5605765 | 6781 | 2: 5668859 | 6786 | 2: 5824441 | 6806 | 2: 6219961 |
| 6807 | 2: 6220338 | 6820 | 2: 6413902 | 6826 | 2: 6665981 | 6827 | 2: 6670529 | 6828 | 2: 6670740 |
| 6829 | 2: 6670761 | 6830 | 2: 6670813 | 6831 | 2: 6670846 | 6842 | 2: 6830098 | 6844 | 2: 6891780 |
| 6846 | 2: 6946227 | 6851 | 2: 7030426 | 6852 | 2: 7053225 | 6855 | 2: 7145809 | 6859 | 2: 7201484 |
| 6860 | 2: 7214741 | 6861 | 2: 7228554 | 6865 | 2: 7278045 | 6867 | 2: 7321689 | 6869 | 2: 7334093 |
| 6870 | 2: 7339046 | 6871 | 2: 7346647 | 6874 | 2: 7389988 | 6876 | 2: 7414723 | 6877 | 2: 7487693 |
| 6880 | 2: 7550253 | 6893 | 2: 7824299 | 6894 | 2: 7854802 | 6895 | 2: 7854894 | 6896 | 2: 7854898 |
| 6903 | 2: 8149483 | 6907 | 2: 8268124 | 6909 | 2: 8273877 | 6925 | 2: 8874962 | 6926 | 2: 8891833 |
| 6948 | 2: 9583773 | 6949 | 2: 9583875 | 6957 | 2: 9791102 | 6960 | 2: 9831264 | 6985 | 2: 10595947 |
| 6986 | 2: 10596946 | 6987 | 2: 10597759 | 6988 | 2: 10603551 | 7032 | 2: 12051677 | 7037 | 2: 12305668 |
| 7043 | 2: 12489693 | 7058 | 2: 13087374 | 7071 | 2: 13360171 | 7072 | 2: 13382039 | 7082 | 2: 13695930 |
| 7084 | 2: 13709033 | 7086 | 2: 13779018 | 7088 | 2: 13797995 | 7090 | 2: 13822009 | 7091 | 2: 13823524 |
| 7092 | 2: 13862800 | 7102 | 2: 14216227 | 7127 | 2: 14784977 | 7139 | 2: 15119742 | 7147 | 2: 15329884 |
| 7157 | 2: 15549082 | 7162 | 2: 15690371 | 7163 | 2: 15693009 | 7164 | 2: 15696748 | 7168 | 2: 15821266 |
| 7173 | 2: 15967374 | 7190 | 2: 16339174 | 7207 | 2: 16790984 | 7211 | 2: 16932422 | 7217 | 2: 17092308 |
| 7223 | 2: 17311769 | 7236 | 2: 17583628 | 7241 | 2: 17659987 | 7242 | 2: 17735524 | 7246 | 2: 17833404 |
| 7250 | 2: 17967333 | 7254 | 2: 18038068 | 7260 | 2: 18147142 | 7261 | 2: 18165858 | 7263 | 2: 18229834 |
| 7264 | 2: 18229859 | 7266 | 2: 18241132 | 7268 | 2: 18244789 | 7270 | 2: 18253097 | 7271 | 2: 18254488 |
| 7273 | 2: 18262337 | 7275 | 2: 18270755 | 7276 | 2: 18271356 | 7277 | 2: 18271358 | 7278 | 2: 18271418 |
| 7279 | 2: 18272565 | 7280 | 2: 18272965 | 7281 | 2: 18275874 | 7282 | 2: 18277584 | 7283 | 2: 18277939 |
| 7284 | 2: 18280757 | 7285 | 2: 18280781 | 7286 | 2: 18282254 | 7287 | 2: 18282552 | 7288 | 2: 18290509 |
| 7289 | 2: 18291940 | 7290 | 2: 18307699 | 7291 | 2: 18323628 | 7293 | 2: 18378637 | 7297 | 2: 18502293 |
| 7304 | 2: 18586886 | 7305 | 2: 18595133 | 7306 | 2: 18605578 | 7310 | 2: 18642606 | 7314 | 2: 18778867 |
| 7316 | 2: 18866785 | 7317 | 2: 18869734 | 7325 | 2: 18981323 | 7326 | 2: 18986957 | 7330 | 2: 19045985 |
| 7335 | 2: 19136987 | 7346 | 2: 19340327 | 7352 | 2: 19534377 | 7355 | 2: 19593435 | 7356 | 2: 19626883 |
| 7357 | 2: 19648311 | 7358 | 2: 19648602 | 7368 | 2: 19897160 | 7369 | 2: 19918403 | 7378 | 2: 20155806 |
| 7380 | 2: 20264485 | 7383 | 2: 20344434 | 7387 | 2: 20427608 | 7394 | 2: 20674260 | 7401 | 2: 20845761 |
| 7403 | 2: 20861585 | 7407 | 2: 21064906 | 7421 | 2: 21401355 | 7422 | 2: 21405561 | 7428 | 2: 21553608 |
| 7438 | 2: 21688312 | 7443 | 2: 21849771 | 7444 | 2: 21867557 | 7445 | 2: 21867592 | 7447 | 2: 21914587 |
| 7449 | 2: 21967376 | 7451 | 2: 22019700 | 7452 | 2: 22051754 | 7458 | 2: 22154085 | 7466 | 2: 22396788 |
| 7472 | 2: 22599197 | 7474 | 2: 22608474 | 7479 | 2: 22767655 | 7480 | 2: 22787231 | 7485 | 2: 23032717 |
| 7489 | 2: 23063150 | 7496 | 2: 23295824 | 7497 | 2: 23312807 | 7502 | 2: 23459486 | 7504 | 2: 23476574 |
| 7505 | 2: 23481265 | 7506 | 2: 23481554 | 7507 | 2: 23481572 | 7509 | 2: 23487782 | 7510 | 2: 23501169 |
| 7511 | 2: 23537225 | 7512 | 2: 23557793 | 7513 | 2: 23558570 | 7520 | 2: 23643567 | 7522 | 2: 23666640 |
| 7523 | 2: 23676088 | 7525 | 2: 23691155 | 7526 | 2: 23696931 | 7527 | 2: 23699667 | 7534 | 2: 23962288 |
| 7536 | 2: 23971606 | 7545 | 2: 24172853 | 7546 | 2: 24176608 | 7552 | 2: 24299477 | 7554 | 2: 24320827 |
| 7555 | 2: 24320845 | 7575 | 2: 24669527 | 7586 | 2: 24895083 | 7591 | 2: 25095591 | 7595 | 2: 25243017 |
| 7597 | 2: 25329638 | 7600 | 2: 25433245 | 7601 | 2: 25441115 | 7602 | 2: 25443814 | 7606 | 2: 25523128 |
| 7607 | 2: 25531340 | 7608 | 2: 25539975 | 7609 | 2: 25556600 | 7617 | 2: 25649108 | 7620 | 2: 25710237 |
| 7658 | 2: 26534630 | 7667 | 2: 26760780 | 7669 | 2: 26762215 | 7670 | 2: 26771611 | 7671 | 2: 26774438 |
| 7674 | 2: 26851479 | 7691 | 2: 27096366 | 7698 | 2: 27312219 | 7702 | 2: 27432834 | 7727 | 2: 27915981 |
| 7736 | 2: 28189986 | 7738 | 2: 28287714 | 7739 | 2: 28287763 | 7740 | 2: 28287812 | 7741 | 2: 28287836 |
| 7746 | 2: 28373427 | 7749 | 2: 28418211 | 7764 | 2: 28631973 | 7780 | 2: 28937935 | 7813 | 2: 29825101 |
| 7817 | 2: 29924141 | 7825 | 2: 30218788 | 7826 | 2: 30225126 | 7827 | 2: 30235162 | 7828 | 2: 30235271 |
| 7837 | 2: 30489470 | 7840 | 2: 30505625 | 7847 | 2: 30620848 | 7850 | 2: 30714513 | 7852 | 2: 30729817 |
| 7867 | 2: 31118343 | 7868 | 2: 31118369 | 7869 | 2: 31121656 | 7887 | 2: 31621372 | 7905 | 2: 32163209 |
| 7921 | 2: 32683358 | 7923 | 2: 32754895 | 7930 | 2: 33004780 | 7937 | 2: 33199953 | 7948 | 2: 33575101 |
| 7949 | 2: 33575122 | 7950 | 2: 33654382 | 7952 | 2: 33671969 | 7953 | 2: 33671986 | 7954 | 2: 33694528 |
| 7959 | 2: 33883065 | 7962 | 2: 33960798 | 7978 | 2: 34316478 | 7981 | 2: 34353723 | 7986 | 2: 34439099 |
| 7989 | 2: 34507044 | 8027 | 2: 35881554 | 8035 | 2: 36123841 | 8036 | 2: 36138834 | 8037 | 2: 36147200 |
| 8051 | 2: 36510607 | 8055 | 2: 36554095 | 8056 | 2: 36613946 | 8057 | 2: 36621379 | 8060 | 2: 36742166 |
| 8075 | 2: 37300847 | 8101 | 2: 38053061 | 8107 | 2: 38237985 | 8108 | 2: 38238121 | 8109 | 2: 38238372 |
| 8110 | 2: 38238415 | 8111 | 2: 38238441 | 8112 | 2: 38238520 | 8113 | 2: 38238541 | 8117 | 2: 38310384 |
| 8121 | 2: 38442344 | 8123 | 2: 38465755 | 8130 | 2: 38600295 | 8131 | 2: 38634299 | 8136 | 2: 38811572 |
| 8163 | 2: 39460697 | 8203 | 2: 40835843 | 8205 | 2: 40867544 | 8206 | 2: 40867584 | 8207 | 2: 40867585 |
| 8208 | 2: 40867624 | 8209 | 2: 40867647 | 8210 | 2: 40867669 | 8211 | 2: 40867710 | 8212 | 2: 40867713 |
| 8215 | 2: 41050502 | 8216 | 2: 41050562 | 8217 | 2: 41050564 | 8218 | 2: 41050613 | 8226 | 2: 41315483 |
| 8227 | 2: 41315504 | 8245 | 2: 41559277 | 8253 | 2: 41775609 | 8254 | 2: 41797511 | 8287 | 2: 42520826 |
| 8288 | 2: 42535114 | 8307 | 2: 43006124 | 8321 | 2: 43491727 | 8323 | 2: 43580317 | 8324 | 2: 43631101 |
| 8328 | 2: 43711459 | 8332 | 2: 43813071 | 8341 | 2: 44152000 | 8343 | 2: 44193529 | 8350 | 2: 44379204 |
| 8351 | 2: 44393093 | 8352 | 2: 44393964 | 8353 | 2: 44403432 | 8356 | 2: 44515259 | 8357 | 2: 44545955 |
| 8358 | 2: 44569313 | 8360 | 2: 44615292 | 8361 | 2: 44621241 | 8367 | 2: 44858072 | 8384 | 2: 45372211 |
| 8388 | 2: 45474859 | 8412 | 2: 45983105 | 8415 | 2: 46033456 | 8420 | 2: 46147458 | 8428 | 2: 46391874 |
| 8434 | 2: 46437983 | 8436 | 2: 46446081 | 8440 | 2: 46534111 | 8446 | 2: 46705643 | 8464 | 2: 47013353 |
| 8467 | 2: 47100164 | 8481 | 2: 47433064 | 8486 | 2: 47596275 | 8491 | 2: 47706783 | 8509 | 2: 48215370 |
| 8522 | 2: 48603698 | 8523 | 2: 48604345 | 8526 | 2: 48755071 | 8528 | 2: 48783617 | 8531 | 2: 48830819 |
| 8534 | 2: 48894232 | 8539 | 2: 48987003 | 8544 | 2: 49067927 | 8555 | 2: 49577101 | 8571 | 2: 50088797 |
| 8590 | 2: 50645993 | 8595 | 2: 50810980 | 8605 | 2: 51033258 | 8615 | 2: 51338525 | 8626 | 2: 51770242 |
| 8628 | 2: 51813793 | 8649 | 2: 52303945 | 8655 | 2: 52440698 | 8660 | 2: 52515449 | 8671 | 2: 52834508 |
| 8675 | 2: 52903006 | 8676 | 2: 52906421 | 8677 | 2: 52909824 | 8681 | 2: 52959066 | 8690 | 2: 53226738 |
| 8735 | 2: 54491736 | 8753 | 2: 54901841 | 8756 | 2: 54993038 | 8757 | 2: 54993081 | 8758 | 2: 54993084 |
| 8759 | 2: 54993089 | 8761 | 2: 55019715 | 8769 | 2: 55150342 | 8770 | 2: 55235366 | 8800 | 2: 56234809 |
| 8802 | 2: 56280951 | 8804 | 2: 56314003 | 8809 | 2: 56469340 | 8816 | 2: 56646269 | 8838 | 2: 57404778 |
| 8839 | 2: 57430549 | 8849 | 2: 57666386 | 8851 | 2: 57700250 | 8858 | 2: 57837920 | 8888 | 2: 58761910 |
| 8890 | 2: 58778117 | 8892 | 2: 58793131 | 8897 | 2: 58929038 | 8899 | 2: 58940947 | 8901 | 2: 58993535 |
| 8903 | 2: 59042027 | 8904 | 2: 59079693 | 8908 | 2: 59202049 | 8909 | 2: 59212187 | 8923 | 2: 59601537 |
| 8931 | 2: 59858246 | 8932 | 2: 59858284 | 8933 | 2: 59858319 | 8934 | 2: 59858339 | 8949 | 2: 60150792 |
| 8967 | 2: 60751699 | 8978 | 2: 61108555 | 8980 | 2: 61144456 | 8981 | 2: 61170888 | 8982 | 2: 61172063 |
| 8985 | 2: 61252370 | 8992 | 2: 61343961 | 8994 | 2: 61449061 | 8995 | 2: 61490160 | 8996 | 2: 61490261 |
| 8998 | 2: 61494582 | 9000 | 2: 61535058 | 9001 | 2: 61535147 | 9002 | 2: 61535149 | 9003 | 2: 61535213 |
| 9013 | 2: 61843883 | 9020 | 2: 62046115 | 9024 | 2: 62302155 | 9046 | 2: 62786824 | 9051 | 2: 62910878 |
| 9052 | 2: 62944872 | 9053 | 2: 62944925 | 9055 | 2: 63002162 | 9065 | 2: 63241642 | 9070 | 2: 63325322 |
| 9078 | 2: 63563631 | 9081 | 2: 63616590 | 9090 | 2: 63926681 | 9124 | 2: 64713810 | 9129 | 2: 64820071 |
| 9148 | 2: 65218951 | 9164 | 2: 65726104 | 9221 | 2: 67528810 | 9226 | 2: 67669505 | 9236 | 2: 68034573 |
| 9267 | 2: 69100324 | 9283 | 2: 69720593 | 9294 | 2: 70008307 | 9303 | 2: 70294251 | 9304 | 2: 70313073 |
| 9310 | 2: 70440178 | 9324 | 2: 70768201 | 9329 | 2: 70931242 | 9334 | 2: 71041850 | 9335 | 2: 71042132 |
| 9336 | 2: 71042190 | 9337 | 2: 71044225 | 9338 | 2: 71095900 | 9341 | 2: 71203819 | 9342 | 2: 71205559 |
| 9349 | 2: 71337903 | 9357 | 2: 71501065 | 9366 | 2: 71912340 | 9375 | 2: 72314326 | 9392 | 2: 72649795 |
| 9393 | 2: 72669915 | 9394 | 2: 72669943 | 9403 | 2: 72918814 | 9404 | 2: 72977278 | 9405 | 2: 72995902 |
| 9407 | 2: 73090036 | 9413 | 2: 73157226 | 9414 | 2: 73160964 | 9415 | 2: 73161983 | 9420 | 2: 73383708 |
| 9422 | 2: 73398069 | 9424 | 2: 73477830 | 9434 | 2: 73757540 | 9437 | 2: 73811343 | 9446 | 2: 73945664 |
| 9449 | 2: 74083815 | 9468 | 2: 74707536 | 9469 | 2: 74707559 | 9470 | 2: 74707612 | 9471 | 2: 74707658 |
| 9472 | 2: 74707710 | 9482 | 2: 75029605 | 9485 | 2: 75173951 | 9493 | 2: 75482167 | 9494 | 2: 75483513 |
| 9501 | 2: 75692826 | 9507 | 2: 75820235 | 9508 | 2: 75834257 | 9517 | 2: 76002206 | 9519 | 2: 76048349 |
| 9527 | 2: 76266636 | 9537 | 2: 76475875 | 9539 | 2: 76503911 | 9556 | 2: 77018992 | 9561 | 2: 77216172 |
| 9563 | 2: 77216223 | 9570 | 2: 77393386 | 9571 | 2: 77423386 | 9575 | 2: 77530544 | 9579 | 2: 77676860 |
| 9580 | 2: 77680269 | 9581 | 2: 77690134 | 9583 | 2: 77796311 | 9590 | 2: 77930065 | 9591 | 2: 77960137 |
| 9592 | 2: 77960148 | 9593 | 2: 77960205 | 9594 | 2: 77960221 | 9595 | 2: 78058933 | 9599 | 2: 78093605 |
| 9608 | 2: 78475004 | 9610 | 2: 78576254 | 9621 | 2: 78787744 | 9622 | 2: 78787785 | 9623 | 2: 78793670 |
| 9625 | 2: 78828241 | 9627 | 2: 78856439 | 9631 | 2: 78919311 | 9632 | 2: 78922277 | 9634 | 2: 78933501 |
| 9635 | 2: 78933506 | 9636 | 2: 78940906 | 9637 | 2: 78942899 | 9640 | 2: 79034805 | 9643 | 2: 79043516 |
| 9665 | 2: 79805247 | 9673 | 2: 79959399 | 9683 | 2: 80095415 | 9686 | 2: 80170719 | 9762 | 2: 83630529 |
| 9763 | 2: 83648977 | 9764 | 2: 83654198 | 9765 | 2: 83657997 | 9766 | 2: 83660404 | 9767 | 2: 83664117 |
| 9768 | 2: 83664826 | 9769 | 2: 83665834 | 9770 | 2: 83667393 | 9771 | 2: 83669899 | 9772 | 2: 83671239 |
| 9774 | 2: 83694268 | 9775 | 2: 83694872 | 9776 | 2: 83696501 | 9777 | 2: 83697095 | 9778 | 2: 83701382 |
| 9779 | 2: 83703221 | 9780 | 2: 83710452 | 9796 | 2: 84314672 | 9801 | 2: 84413080 | 9806 | 2: 84544851 |
| 9807 | 2: 84554472 | 9808 | 2: 84556946 | 9809 | 2: 84558765 | 9810 | 2: 84558813 | 9811 | 2: 84579866 |
| 9812 | 2: 84579884 | 9814 | 2: 84594590 | 9815 | 2: 84594621 | 9816 | 2: 84631977 | 9821 | 2: 84747533 |
| 9826 | 2: 84895919 | 9829 | 2: 84986688 | 9841 | 2: 85268807 | 9842 | 2: 85302489 | 9857 | 2: 85748841 |
| 9868 | 2: 85921857 | 9870 | 2: 85984791 | 9871 | 2: 86008072 | 9876 | 2: 86150518 | 9881 | 2: 86310570 |
| 9882 | 2: 86387502 | 9886 | 2: 86419348 | 9889 | 2: 86436688 | 9891 | 2: 86469285 | 9894 | 2: 86509868 |
| 9961 | 2: 88510673 | 9965 | 2: 88623312 | 9970 | 2: 88707441 | 9971 | 2: 88719162 | 9973 | 2: 88746032 |
| 9975 | 2: 88782810 | 9977 | 2: 88859157 | 9983 | 2: 89070767 | 9984 | 2: 89076240 | 9985 | 2: 89084301 |
| 9986 | 2: 89086388 | 9987 | 2: 89095014 | 9988 | 2: 89100826 | 9989 | 2: 89104328 | 9990 | 2: 89109055 |
| 9991 | 2: 89114149 | 9992 | 2: 89117889 | 9993 | 2: 89119778 | 9994 | 2: 89122696 | 9995 | 2: 89126698 |
| 9996 | 2: 89128778 | 9997 | 2: 89129756 | 9998 | 2: 89130672 | 9999 | 2: 89131642 | 10000 | 2: 89132131 |
| 10001 | 2: 89132635 | 10002 | 2: 89133746 | 10003 | 2: 89136358 | 10004 | 2: 89136948 | 10005 | 2: 89143985 |
| 10006 | 2: 89150132 | 10007 | 2: 89157341 | 10008 | 2: 89161844 | 10009 | 2: 89164769 | 10010 | 2: 89172177 |
| 10011 | 2: 89183562 | 10012 | 2: 89188097 | 10013 | 2: 89192815 | 10014 | 2: 89196778 | 10015 | 2: 89206007 |
| 10016 | 2: 89210274 | 10017 | 2: 89220562 | 10021 | 2: 89293059 | 10022 | 2: 89329459 | 10023 | 2: 89330715 |
| 10024 | 2: 89332510 | 10025 | 2: 89333883 | 10026 | 2: 89335072 | 10028 | 2: 89337942 | 10031 | 2: 89377233 |
| 10034 | 2: 89445178 | 10049 | 2: 89808466 | 10050 | 2: 89813015 | 10052 | 2: 89841096 | 10054 | 2: 89876885 |
| 10055 | 2: 89878690 | 10057 | 2: 89908253 | 10059 | 2: 89916678 | 10060 | 2: 89948655 | 10061 | 2: 89952700 |
| 10062 | 2: 89952808 | 10063 | 2: 89953249 | 10064 | 2: 89964009 | 10065 | 2: 89964459 | 10066 | 2: 89964908 |
| 10100 | 2: 91309327 | 10101 | 2: 91319564 | 10123 | 2: 92012717 | 10131 | 2: 92095096 | 10132 | 2: 92109122 |
| 10142 | 2: 92406522 | 10143 | 2: 92416012 | 10146 | 2: 92501151 | 10148 | 2: 92504237 | 10154 | 2: 92593249 |
| 10157 | 2: 92677123 | 10158 | 2: 92688749 | 10163 | 2: 92792925 | 10165 | 2: 92800370 | 10167 | 2: 92859552 |
| 10168 | 2: 92875951 | 10171 | 2: 93007619 | 10197 | 2: 93432778 | 10217 | 2: 93888418 | 10231 | 2: 94155809 |
| 10233 | 2: 94177314 | 10234 | 2: 94238285 | 10238 | 2: 94284858 | 10242 | 2: 94408041 | 10244 | 2: 94532812 |
| 10250 | 2: 94662652 | 10263 | 2: 94943212 | 10269 | 2: 95104701 | 10273 | 2: 95191751 | 10276 | 2: 95262942 |
| 10277 | 2: 95265410 | 10297 | 2: 95781931 | 10298 | 2: 95788314 | 10299 | 2: 95797570 | 10304 | 2: 95957388 |
| 10307 | 2: 96027434 | 10312 | 2: 96139223 | 10313 | 2: 96162249 | 10321 | 2: 96318089 | 10324 | 2: 96352046 |
| 10330 | 2: 96463623 | 10332 | 2: 96505449 | 10333 | 2: 96512869 | 10334 | 2: 96514006 | 10335 | 2: 96514035 |
| 10336 | 2: 96514066 | 10337 | 2: 96514071 | 10339 | 2: 96516949 | 10340 | 2: 96517000 | 10341 | 2: 96518404 |
| 10342 | 2: 96519726 | 10343 | 2: 96519764 | 10344 | 2: 96519823 | 10345 | 2: 96522707 | 10346 | 2: 96522837 |
| 10352 | 2: 96721652 | 10373 | 2: 97569993 | 10393 | 2: 98098074 | 10394 | 2: 98098574 | 10397 | 2: 98189476 |
| 10411 | 2: 98434332 | 10435 | 2: 99027387 | 10443 | 2: 99309195 | 10444 | 2: 99310709 | 10477 | 2: 100060201 |
| 10486 | 2: 100359058 | 10488 | 2: 100436598 | 10489 | 2: 100436640 | 10490 | 2: 100507665 | 10491 | 2: 100527030 |
| 10508 | 2: 100969194 | 10512 | 2: 101142510 | 10515 | 2: 101176628 | 10516 | 2: 101267741 | 10522 | 2: 101470754 |
| 10524 | 2: 101503694 | 10526 | 2: 101544902 | 10529 | 2: 101696463 | 10540 | 2: 102030097 | 10541 | 2: 102031180 |
| 10567 | 2: 102763970 | 10569 | 2: 102854361 | 10570 | 2: 102861535 | 10574 | 2: 103021453 | 10575 | 2: 103022409 |
| 10577 | 2: 103050047 | 10578 | 2: 103087349 | 10588 | 2: 103396204 | 10589 | 2: 103402752 | 10592 | 2: 103445587 |
| 10595 | 2: 103517827 | 10598 | 2: 103593199 | 10610 | 2: 103884686 | 10615 | 2: 104008673 | 10622 | 2: 104115856 |
| 10628 | 2: 104308034 | 10635 | 2: 104472870 | 10641 | 2: 104678498 | 10643 | 2: 104790859 | 10644 | 2: 104814826 |
| 10646 | 2: 104850637 | 10647 | 2: 104882698 | 10660 | 2: 105042627 | 10669 | 2: 105160301 | 10679 | 2: 105493554 |
| 10682 | 2: 105532073 | 10684 | 2: 105558282 | 10685 | 2: 105589302 | 10689 | 2: 105726409 | 10697 | 2: 105850938 |
| 10698 | 2: 105883087 | 10702 | 2: 105943923 | 10704 | 2: 106005198 | 10705 | 2: 106040867 | 10708 | 2: 106093074 |
| 10709 | 2: 106134401 | 10716 | 2: 106279517 | 10718 | 2: 106396433 | 10719 | 2: 106446238 | 10720 | 2: 106455619 |
| 10722 | 2: 106582615 | 10724 | 2: 106599852 | 10725 | 2: 106602909 | 10726 | 2: 106605195 | 10727 | 2: 106609300 |
| 10728 | 2: 106609491 | 10729 | 2: 106615459 | 10730 | 2: 106624131 | 10731 | 2: 106632743 | 10732 | 2: 106635677 |
| 10740 | 2: 106788337 | 10748 | 2: 106865812 | 10750 | 2: 106960370 | 10752 | 2: 107059040 | 10753 | 2: 107076804 |
| 10766 | 2: 107558581 | 10768 | 2: 107560640 | 10769 | 2: 107560655 | 10773 | 2: 107620491 | 10774 | 2: 107628799 |
| 10777 | 2: 107692575 | 10780 | 2: 107718206 | 10793 | 2: 107772997 | 10796 | 2: 107824497 | 10797 | 2: 107827321 |
| 10799 | 2: 107881110 | 10808 | 2: 108144100 | 10810 | 2: 108165798 | 10820 | 2: 108449215 | 10827 | 2: 108649909 |
| 10835 | 2: 108855822 | 10838 | 2: 108918709 | 10839 | 2: 108932264 | 10841 | 2: 108944061 | 10850 | 2: 109135684 |
| 10861 | 2: 109403406 | 10862 | 2: 109415989 | 10867 | 2: 109507997 | 10868 | 2: 109525518 | 10871 | 2: 109605505 |
| 10874 | 2: 109627420 | 10878 | 2: 109678083 | 10896 | 2: 110207989 | 10909 | 2: 110556883 | 10919 | 2: 111069391 |
| 10923 | 2: 111211200 | 10924 | 2: 111216842 | 10931 | 2: 111473451 | 10946 | 2: 111903128 | 10950 | 2: 112002693 |
| 10951 | 2: 112064672 | 10952 | 2: 112068968 | 10963 | 2: 112309161 | 10982 | 2: 112824303 | 10985 | 2: 112865726 |
| 10988 | 2: 112884446 | 10996 | 2: 112999914 | 10998 | 2: 113039809 | 11000 | 2: 113071684 | 11004 | 2: 113217166 |
| 11005 | 2: 113217179 | 11006 | 2: 113217208 | 11007 | 2: 113217254 | 11033 | 2: 113775291 | 11040 | 2: 113927720 |
| 11052 | 2: 114312086 | 11056 | 2: 114441340 | 11057 | 2: 114511997 | 11070 | 2: 114795811 | 11072 | 2: 114857637 |
| 11087 | 2: 115277541 | 11088 | 2: 115279258 | 11092 | 2: 115316715 | 11096 | 2: 115405473 | 11097 | 2: 115455721 |
| 11106 | 2: 115621193 | 11108 | 2: 115625826 | 11125 | 2: 115953383 | 11155 | 2: 116733465 | 11161 | 2: 116888348 |
| 11167 | 2: 117033411 | 11168 | 2: 117047832 | 11169 | 2: 117079799 | 11172 | 2: 117133057 | 11178 | 2: 117240992 |
| 11187 | 2: 117466579 | 11195 | 2: 117945652 | 11200 | 2: 118099830 | 11207 | 2: 118201421 | 11220 | 2: 118509097 |
| 11222 | 2: 118554241 | 11239 | 2: 119145772 | 11240 | 2: 119196348 | 11241 | 2: 119198191 | 11242 | 2: 119199613 |
| 11244 | 2: 119244130 | 11245 | 2: 119264906 | 11248 | 2: 119291176 | 11252 | 2: 119322052 | 11257 | 2: 119465613 |
| 11260 | 2: 119511934 | 11261 | 2: 119512418 | 11262 | 2: 119522741 | 11263 | 2: 119526528 | 11264 | 2: 119533194 |
| 11267 | 2: 119548536 | 11269 | 2: 119562713 | 11270 | 2: 119564586 | 11271 | 2: 119565180 | 11272 | 2: 119566000 |
| 11273 | 2: 119576736 | 11277 | 2: 119825482 | 11278 | 2: 119862058 | 11290 | 2: 120178985 | 11292 | 2: 120195958 |
| 11293 | 2: 120199797 | 11308 | 2: 120568141 | 11310 | 2: 120671914 | 11319 | 2: 120900260 | 11322 | 2: 120932791 |
| 11331 | 2: 121151913 | 11333 | 2: 121188721 | 11352 | 2: 121755672 | 11358 | 2: 121893423 | 11359 | 2: 121894763 |
| 11375 | 2: 122580635 | 11382 | 2: 122800513 | 11385 | 2: 122878392 | 11393 | 2: 123070777 | 11394 | 2: 123070905 |
| 11395 | 2: 123070944 | 11396 | 2: 123100345 | 11397 | 2: 123100370 | 11414 | 2: 123539540 | 11415 | 2: 123539541 |
| 11417 | 2: 123562349 | 11419 | 2: 123590694 | 11420 | 2: 123590830 | 11421 | 2: 123610591 | 11427 | 2: 123692694 |
| 11447 | 2: 124332744 | 11453 | 2: 124600152 | 11458 | 2: 124710998 | 11462 | 2: 124913543 | 11470 | 2: 125166728 |
| 11476 | 2: 125250396 | 11479 | 2: 125277549 | 11480 | 2: 125286027 | 11481 | 2: 125349483 | 11490 | 2: 125625209 |
| 11492 | 2: 125653640 | 11493 | 2: 125653952 | 11496 | 2: 125742333 | 11499 | 2: 125810128 | 11502 | 2: 125931055 |
| 11503 | 2: 125952736 | 11508 | 2: 126114829 | 11511 | 2: 126173762 | 11515 | 2: 126267198 | 11516 | 2: 126267881 |
| 11517 | 2: 126273576 | 11518 | 2: 126275683 | 11519 | 2: 126286517 | 11522 | 2: 126366193 | 11523 | 2: 126367550 |
| 11525 | 2: 126372344 | 11526 | 2: 126380236 | 11529 | 2: 126489135 | 11575 | 2: 127003970 | 11576 | 2: 127004702 |
| 11583 | 2: 127018411 | 11587 | 2: 127027686 | 11589 | 2: 127038523 | 11606 | 2: 127110467 | 11613 | 2: 127238953 |
| 11623 | 2: 127397394 | 11629 | 2: 127525976 | 11634 | 2: 127633593 | 11636 | 2: 127635918 | 11637 | 2: 127639135 |
| 11654 | 2: 128049090 | 11656 | 2: 128099391 | 11672 | 2: 128508443 | 11673 | 2: 128508479 | 11677 | 2: 128603208 |
| 11683 | 2: 128718168 | 11688 | 2: 128782288 | 11691 | 2: 128791601 | 11692 | 2: 128804408 | 11695 | 2: 128916878 |
| 11701 | 2: 129068395 | 11702 | 2: 129073892 | 11704 | 2: 129084385 | 11705 | 2: 129095315 | 11706 | 2: 129098350 |
| 11707 | 2: 129101237 | 11719 | 2: 129414787 | 11721 | 2: 129451897 | 11722 | 2: 129453738 | 11723 | 2: 129463584 |
| 11724 | 2: 129475673 | 11725 | 2: 129514375 | 11735 | 2: 129823411 | 11740 | 2: 129952968 | 11750 | 2: 130179342 |
| 11751 | 2: 130179458 | 11753 | 2: 130297720 | 11759 | 2: 130405723 | 11761 | 2: 130416105 | 11771 | 2: 130763239 |
| 11773 | 2: 130778311 | 11775 | 2: 130786713 | 11777 | 2: 130887344 | 11779 | 2: 130922023 | 11780 | 2: 130922555 |
| 11781 | 2: 130924042 | 11786 | 2: 131056297 | 11788 | 2: 131116831 | 11792 | 2: 131182393 | 11793 | 2: 131183245 |
| 11799 | 2: 131239243 | 11810 | 2: 131495314 | 11812 | 2: 131522458 | 11818 | 2: 131759255 | 11827 | 2: 131997425 |
| 11828 | 2: 132032080 | 11829 | 2: 132052956 | 11836 | 2: 132152838 | 11850 | 2: 132304236 | 11856 | 2: 132453956 |
| 11862 | 2: 132630596 | 11873 | 2: 132828927 | 11874 | 2: 132829391 | 11883 | 2: 133050393 | 11884 | 2: 133069759 |
| 11886 | 2: 133077482 | 11888 | 2: 133094549 | 11889 | 2: 133095512 | 11892 | 2: 133207446 | 11897 | 2: 133305481 |
| 11901 | 2: 133364775 | 11905 | 2: 133421233 | 11908 | 2: 133536795 | 11910 | 2: 133542411 | 11915 | 2: 133623325 |
| 11923 | 2: 133694191 | 11927 | 2: 133778125 | 11928 | 2: 133778616 | 11929 | 2: 133785516 | 11931 | 2: 133786535 |
| 11932 | 2: 133787224 | 11933 | 2: 133787956 | 11934 | 2: 133787982 | 11935 | 2: 133788783 | 11957 | 2: 134106505 |
| 11963 | 2: 134189401 | 11964 | 2: 134189458 | 11965 | 2: 134190122 | 11966 | 2: 134190219 | 11977 | 2: 134376164 |
| 11979 | 2: 134438884 | 11982 | 2: 134480181 | 11983 | 2: 134480249 | 11984 | 2: 134480295 | 11985 | 2: 134480304 |
| 11988 | 2: 134508261 | 11997 | 2: 134616684 | 11998 | 2: 134616699 | 12008 | 2: 134803486 | 12012 | 2: 134834209 |
| 12013 | 2: 134843772 | 12019 | 2: 134917533 | 12020 | 2: 134917563 | 12021 | 2: 134917597 | 12022 | 2: 134917643 |
| 12023 | 2: 134917658 | 12031 | 2: 135091858 | 12033 | 2: 135100100 | 12035 | 2: 135114924 | 12038 | 2: 135166182 |
| 12039 | 2: 135171128 | 12042 | 2: 135233030 | 12043 | 2: 135235158 | 12048 | 2: 135319271 | 12054 | 2: 135454017 |
| 12058 | 2: 135514495 | 12063 | 2: 135598504 | 12064 | 2: 135598610 | 12066 | 2: 135604349 | 12069 | 2: 135646413 |
| 12074 | 2: 135709576 | 12076 | 2: 135727874 | 12077 | 2: 135727928 | 12079 | 2: 135837161 | 12080 | 2: 135837263 |
| 12082 | 2: 135865945 | 12083 | 2: 135887710 | 12084 | 2: 135900620 | 12085 | 2: 135900833 | 12086 | 2: 135904261 |
| 12087 | 2: 135908217 | 12091 | 2: 136093517 | 12092 | 2: 136093612 | 12104 | 3: 197807 | 12127 | 3: 746327 |
| 12135 | 3: 867632 | 12139 | 3: 923989 | 12140 | 3: 924352 | 12142 | 3: 941811 | 12146 | 3: 969297 |
| 12155 | 3: 1114174 | 12162 | 3: 1183922 | 12181 | 3: 1651460 | 12184 | 3: 1672587 | 12188 | 3: 1729118 |
| 12195 | 3: 1939081 | 12198 | 3: 1965533 | 12199 | 3: 1967307 | 12204 | 3: 2023241 | 12206 | 3: 2062805 |
| 12208 | 3: 2064509 | 12211 | 3: 2089682 | 12212 | 3: 2098556 | 12213 | 3: 2104449 | 12235 | 3: 2740113 |
| 12243 | 3: 2986445 | 12245 | 3: 2993307 | 12250 | 3: 3052396 | 12283 | 3: 3863731 | 12305 | 3: 4250424 |
| 12306 | 3: 4264560 | 12307 | 3: 4280470 | 12308 | 3: 4285570 | 12310 | 3: 4298685 | 12312 | 3: 4313521 |
| 12314 | 3: 4363949 | 12316 | 3: 4398654 | 12332 | 3: 4566642 | 12335 | 3: 4593309 | 12338 | 3: 4692667 |
| 12344 | 3: 4732630 | 12345 | 3: 4732659 | 12354 | 3: 4874925 | 12355 | 3: 4874954 | 12373 | 3: 5268053 |
| 12377 | 3: 5309746 | 12383 | 3: 5436199 | 12384 | 3: 5462355 | 12408 | 3: 5998482 | 12422 | 3: 6207469 |
| 12424 | 3: 6282733 | 12425 | 3: 6293053 | 12432 | 3: 6517775 | 12443 | 3: 6741890 | 12444 | 3: 6771925 |
| 12445 | 3: 6771928 | 12446 | 3: 6771969 | 12447 | 3: 6772038 | 12469 | 3: 7060982 | 12470 | 3: 7106174 |
| 12478 | 3: 7235310 | 12479 | 3: 7238069 | 12481 | 3: 7255164 | 12483 | 3: 7357130 | 12486 | 3: 7383219 |
| 12492 | 3: 7455499 | 12506 | 3: 7653471 | 12511 | 3: 7783911 | 12512 | 3: 7822809 | 12514 | 3: 7838838 |
| 12522 | 3: 7931685 | 12523 | 3: 7931828 | 12524 | 3: 7931887 | 12528 | 3: 7961056 | 12537 | 3: 8147516 |
| 12538 | 3: 8163086 | 12539 | 3: 8174824 | 12540 | 3: 8183497 | 12541 | 3: 8184872 | 12542 | 3: 8186958 |
| 12543 | 3: 8193625 | 12544 | 3: 8194122 | 12545 | 3: 8208066 | 12551 | 3: 8277038 | 12552 | 3: 8300299 |
| 12555 | 3: 8337519 | 12570 | 3: 8661008 | 12584 | 3: 9427220 | 12587 | 3: 9485193 | 12588 | 3: 9485906 |
| 12590 | 3: 9513246 | 12594 | 3: 9564986 | 12595 | 3: 9591739 | 12598 | 3: 9636755 | 12605 | 3: 9764080 |
| 12606 | 3: 9832615 | 12608 | 3: 9847485 | 12609 | 3: 9858051 | 12612 | 3: 9863664 | 12622 | 3: 10118782 |
| 12623 | 3: 10118907 | 12624 | 3: 10119257 | 12625 | 3: 10119319 | 12626 | 3: 10122375 | 12627 | 3: 10122970 |
| 12628 | 3: 10123769 | 12629 | 3: 10128298 | 12630 | 3: 10132847 | 12631 | 3: 10133516 | 12632 | 3: 10134377 |
| 12633 | 3: 10136175 | 12634 | 3: 10137707 | 12635 | 3: 10140467 | 12636 | 3: 10141169 | 12637 | 3: 10156326 |
| 12638 | 3: 10159182 | 12639 | 3: 10177781 | 12641 | 3: 10178026 | 12642 | 3: 10178076 | 12643 | 3: 10178165 |
| 12644 | 3: 10178365 | 12645 | 3: 10178537 | 12652 | 3: 10343580 | 12656 | 3: 10373626 | 12662 | 3: 10458829 |
| 12663 | 3: 10458901 | 12664 | 3: 10458904 | 12665 | 3: 10458919 | 12666 | 3: 10458930 | 12667 | 3: 10458938 |
| 12668 | 3: 10459027 | 12669 | 3: 10459091 | 12670 | 3: 10459139 | 12671 | 3: 10459202 | 12672 | 3: 10459244 |
| 12673 | 3: 10459247 | 12674 | 3: 10459248 | 12675 | 3: 10459299 | 12676 | 3: 10459407 | 12677 | 3: 10459424 |
| 12679 | 3: 10497816 | 12680 | 3: 10497887 | 12681 | 3: 10497945 | 12682 | 3: 10497948 | 12683 | 3: 10497963 |
| 12684 | 3: 10498011 | 12686 | 3: 10519036 | 12688 | 3: 10519145 | 12689 | 3: 10526199 | 12690 | 3: 10526562 |
| 12691 | 3: 10526604 | 12692 | 3: 10527562 | 12693 | 3: 10527602 | 12694 | 3: 10527675 | 12700 | 3: 10725165 |
| 12703 | 3: 10854110 | 12704 | 3: 10854114 | 12705 | 3: 10854133 | 12706 | 3: 10854173 | 12707 | 3: 10854726 |
| 12708 | 3: 10854773 | 12710 | 3: 10868768 | 12738 | 3: 11492432 | 12739 | 3: 11524924 | 12740 | 3: 11525352 |
| 12741 | 3: 11525876 | 12744 | 3: 11607381 | 12746 | 3: 11634350 | 12747 | 3: 11634398 | 12748 | 3: 11635619 |
| 12762 | 3: 12154189 | 12786 | 3: 12876946 | 12792 | 3: 13018051 | 12799 | 3: 13164663 | 12815 | 3: 13606552 |
| 12818 | 3: 13623598 | 12819 | 3: 13623632 | 12820 | 3: 13624757 | 12829 | 3: 13851188 | 12839 | 3: 13973650 |
| 12841 | 3: 13997525 | 12844 | 3: 14032792 | 12848 | 3: 14146757 | 12849 | 3: 14158851 | 12850 | 3: 14172051 |
| 12851 | 3: 14184603 | 12852 | 3: 14185315 | 12853 | 3: 14185422 | 12854 | 3: 14188705 | 12857 | 3: 14233203 |
| 12858 | 3: 14239901 | 12859 | 3: 14259325 | 12862 | 3: 14312950 | 12867 | 3: 14498473 | 12868 | 3: 14500940 |
| 12870 | 3: 14546904 | 12874 | 3: 14640547 | 12877 | 3: 14754130 | 12879 | 3: 14774051 | 12883 | 3: 14797864 |
| 12888 | 3: 14853239 | 12891 | 3: 14983274 | 12895 | 3: 15110207 | 12897 | 3: 15181630 | 12899 | 3: 15254497 |
| 12904 | 3: 15408885 | 12905 | 3: 15410634 | 12906 | 3: 15410653 | 12907 | 3: 15410660 | 12911 | 3: 15464742 |
| 12915 | 3: 15536943 | 12917 | 3: 15587630 | 12921 | 3: 15632301 | 12923 | 3: 15659384 | 12929 | 3: 15702216 |
| 12932 | 3: 15793894 | 12933 | 3: 15794524 | 12945 | 3: 15926790 | 12950 | 3: 16002303 | 12955 | 3: 16101556 |
| 12956 | 3: 16101690 | 12957 | 3: 16153064 | 12961 | 3: 16228126 | 12964 | 3: 16347204 | 12978 | 3: 16700799 |
| 12979 | 3: 16719574 | 12980 | 3: 16719945 | 12981 | 3: 16720763 | 12982 | 3: 16720823 | 12983 | 3: 16720842 |
| 12984 | 3: 16721218 | 12985 | 3: 16723559 | 12986 | 3: 16725242 | 13003 | 3: 17249309 | 13035 | 3: 18290761 |
| 13058 | 3: 18945520 | 13061 | 3: 19021728 | 13063 | 3: 19054577 | 13070 | 3: 19211257 | 13071 | 3: 19217706 |
| 13072 | 3: 19217713 | 13073 | 3: 19218362 | 13074 | 3: 19225387 | 13075 | 3: 19225406 | 13076 | 3: 19225421 |
| 13077 | 3: 19225424 | 13078 | 3: 19225426 | 13079 | 3: 19225509 | 13082 | 3: 19277608 | 13089 | 3: 19513415 |
| 13094 | 3: 19685974 | 13097 | 3: 19711385 | 13099 | 3: 19775942 | 13101 | 3: 19816181 | 13109 | 3: 20143190 |
| 13110 | 3: 20145668 | 13112 | 3: 20160583 | 13116 | 3: 20338589 | 13123 | 3: 20549806 | 13125 | 3: 20564672 |
| 13130 | 3: 20805323 | 13132 | 3: 20877656 | 13142 | 3: 21424575 | 13168 | 3: 22009877 | 13173 | 3: 22059782 |
| 13178 | 3: 22108825 | 13201 | 3: 22592195 | 13206 | 3: 22780511 | 13207 | 3: 22808543 | 13209 | 3: 22841288 |
| 13211 | 3: 22869028 | 13214 | 3: 22938448 | 13215 | 3: 22963089 | 13219 | 3: 23051090 | 13220 | 3: 23087313 |
| 13225 | 3: 23186080 | 13231 | 3: 23299798 | 13233 | 3: 23367141 | 13235 | 3: 23401470 | 13242 | 3: 23543571 |
| 13259 | 3: 24023430 | 13260 | 3: 24023439 | 13265 | 3: 24145693 | 13266 | 3: 24151362 | 13269 | 3: 24196352 |
| 13270 | 3: 24213490 | 13272 | 3: 24238856 | 13281 | 3: 24394654 | 13283 | 3: 24424423 | 13301 | 3: 24950077 |
| 13308 | 3: 25150752 | 13310 | 3: 25164615 | 13311 | 3: 25170221 | 13312 | 3: 25173303 | 13331 | 3: 25597219 |
| 13332 | 3: 25600844 | 13344 | 3: 26002177 | 13346 | 3: 26027856 | 13347 | 3: 26036214 | 13348 | 3: 26044741 |
| 13349 | 3: 26046291 | 13359 | 3: 26212407 | 13360 | 3: 26212494 | 13361 | 3: 26212691 | 13363 | 3: 26256361 |
| 13371 | 3: 26380532 | 13372 | 3: 26391670 | 13377 | 3: 26439980 | 13378 | 3: 26469071 | 13379 | 3: 26473706 |
| 13391 | 3: 26893372 | 13395 | 3: 26962431 | 13397 | 3: 26979935 | 13399 | 3: 27052772 | 13400 | 3: 27057546 |
| 13401 | 3: 27081349 | 13409 | 3: 27180648 | 13413 | 3: 27264556 | 13414 | 3: 27294196 | 13417 | 3: 27380069 |
| 13418 | 3: 27382883 | 13419 | 3: 27384412 | 13423 | 3: 27481652 | 13452 | 3: 27642990 | 13454 | 3: 27663059 |
| 13458 | 3: 27753499 | 13459 | 3: 27756989 | 13463 | 3: 27871144 | 13465 | 3: 27916123 | 13468 | 3: 27942850 |
| 13469 | 3: 27942868 | 13472 | 3: 27986412 | 13473 | 3: 27990865 | 13476 | 3: 28061404 | 13495 | 3: 28713706 |
| 13509 | 3: 29019861 | 13537 | 3: 29488699 | 13545 | 3: 29585223 | 13546 | 3: 29592827 | 13547 | 3: 29597761 |
| 13548 | 3: 29601864 | 13550 | 3: 29625965 | 13555 | 3: 29681833 | 13558 | 3: 29705190 | 13559 | 3: 29705342 |
| 13560 | 3: 29705512 | 13561 | 3: 29706025 | 13562 | 3: 29706295 | 13563 | 3: 29706336 | 13568 | 3: 29857727 |
| 13570 | 3: 29915442 | 13571 | 3: 29944912 | 13573 | 3: 29954086 | 13575 | 3: 29989198 | 13588 | 3: 30363529 |
| 13607 | 3: 30832758 | 13613 | 3: 30890540 | 13619 | 3: 31013256 | 13622 | 3: 31065769 | 13627 | 3: 31222103 |
| 13630 | 3: 31343441 | 13645 | 3: 31640573 | 13646 | 3: 31689225 | 13652 | 3: 31856497 | 13654 | 3: 31864015 |
| 13655 | 3: 31867053 | 13657 | 3: 31896363 | 13664 | 3: 32046129 | 13665 | 3: 32047685 | 13666 | 3: 32079966 |
| 13668 | 3: 32103555 | 13671 | 3: 32156925 | 13673 | 3: 32164330 | 13676 | 3: 32278723 | 13681 | 3: 32512486 |
| 13699 | 3: 32780141 | 13738 | 3: 32985901 | 13743 | 3: 33003020 | 13762 | 3: 33254753 | 13780 | 3: 33545907 |
| 13781 | 3: 33549574 | 13784 | 3: 33581206 | 13786 | 3: 33623020 | 13787 | 3: 33626853 | 13788 | 3: 33628444 |
| 13789 | 3: 33635163 | 13790 | 3: 33641481 | 13791 | 3: 33644276 | 13792 | 3: 33647252 | 13793 | 3: 33648187 |
| 13794 | 3: 33652206 | 13795 | 3: 33655047 | 13796 | 3: 33667890 | 13797 | 3: 33668793 | 13798 | 3: 33673078 |
| 13799 | 3: 33679729 | 13800 | 3: 33680460 | 13806 | 3: 33800750 | 13810 | 3: 33917904 | 13812 | 3: 33969180 |
| 13814 | 3: 33975547 | 13819 | 3: 34094670 | 13828 | 3: 34304343 | 13832 | 3: 34398092 | 13839 | 3: 34520818 |
| 13840 | 3: 34541029 | 13841 | 3: 34544910 | 13844 | 3: 34626494 | 13845 | 3: 34639336 | 13846 | 3: 34639367 |
| 13847 | 3: 34639414 | 13848 | 3: 34643371 | 13849 | 3: 34644168 | 13850 | 3: 34644227 | 13851 | 3: 34644230 |
| 13852 | 3: 34644292 | 13853 | 3: 34644311 | 13854 | 3: 34646033 | 13855 | 3: 34648675 | 13856 | 3: 34648858 |
| 13857 | 3: 34654205 | 13858 | 3: 34702016 | 13862 | 3: 34749792 | 13865 | 3: 34794868 | 13871 | 3: 34932689 |
| 13872 | 3: 34999359 | 13880 | 3: 35139460 | 13881 | 3: 35139484 | 13886 | 3: 35172154 | 13889 | 3: 35229235 |
| 13892 | 3: 35308462 | 13902 | 3: 35598523 | 13905 | 3: 35702543 | 13906 | 3: 35714205 | 13910 | 3: 35853066 |
| 13914 | 3: 35918054 | 13919 | 3: 36000811 | 13923 | 3: 36106918 | 13924 | 3: 36139522 | 13930 | 3: 36276403 |
| 13937 | 3: 36323043 | 13940 | 3: 36402532 | 13941 | 3: 36414305 | 13950 | 3: 36709252 | 13951 | 3: 36709316 |
| 13958 | 3: 36906114 | 13985 | 3: 37656931 | 13986 | 3: 37657062 | 13991 | 3: 37904335 | 14029 | 3: 39458322 |
| 14043 | 3: 39836580 | 14050 | 3: 40045551 | 14052 | 3: 40104674 | 14057 | 3: 40188225 | 14075 | 3: 40698452 |
| 14081 | 3: 41015843 | 14084 | 3: 41043374 | 14088 | 3: 41141556 | 14093 | 3: 41367055 | 14097 | 3: 41491123 |
| 14100 | 3: 41527680 | 14101 | 3: 41534229 | 14103 | 3: 41589506 | 14104 | 3: 41589571 | 14107 | 3: 41614043 |
| 14113 | 3: 41934611 | 14114 | 3: 41939570 | 14115 | 3: 41940253 | 14117 | 3: 41944095 | 14118 | 3: 41945371 |
| 14119 | 3: 41946261 | 14124 | 3: 42082768 | 14136 | 3: 42496233 | 14137 | 3: 42502863 | 14140 | 3: 42614221 |
| 14141 | 3: 42635738 | 14143 | 3: 42650352 | 14144 | 3: 42659935 | 14148 | 3: 42725238 | 14152 | 3: 42769882 |
| 14155 | 3: 42840302 | 14156 | 3: 42872218 | 14159 | 3: 42900902 | 14160 | 3: 42978663 | 14165 | 3: 43002941 |
| 14167 | 3: 43038774 | 14175 | 3: 43325595 | 14176 | 3: 43325725 | 14177 | 3: 43334230 | 14182 | 3: 43389979 |
| 14183 | 3: 43397421 | 14185 | 3: 43426451 | 14193 | 3: 43680161 | 14194 | 3: 43696024 | 14195 | 3: 43698262 |
| 14196 | 3: 43698948 | 14203 | 3: 43873693 | 14206 | 3: 44017413 | 14215 | 3: 44305690 | 14226 | 3: 44620511 |
| 14235 | 3: 44902917 | 14238 | 3: 44948682 | 14260 | 3: 45663768 | 14298 | 3: 46955918 | 14299 | 3: 46978051 |
| 14309 | 3: 47252028 | 14315 | 3: 47485150 | 14333 | 3: 47811507 | 14352 | 3: 48155117 | 14353 | 3: 48159568 |
| 14363 | 3: 48456906 | 14367 | 3: 48577665 | 14372 | 3: 48681437 | 14374 | 3: 48741921 | 14385 | 3: 48923622 |
| 14390 | 3: 48954466 | 14403 | 3: 49283986 | 14413 | 3: 49441533 | 14414 | 3: 49442661 | 14417 | 3: 49485613 |
| 14418 | 3: 49491032 | 14419 | 3: 49499289 | 14474 | 3: 49732105 | 14477 | 3: 49835535 | 14478 | 3: 49879024 |
| 14479 | 3: 49897610 | 14501 | 3: 50633952 | 14516 | 3: 51122225 | 14527 | 3: 51610699 | 14530 | 3: 51645901 |
| 14532 | 3: 51683713 | 14534 | 3: 51740729 | 14535 | 3: 51755409 | 14603 | 3: 53343252 | 14616 | 3: 53703853 |
| 14626 | 3: 54144822 | 14628 | 3: 54149746 | 14629 | 3: 54167611 | 14630 | 3: 54178592 | 14631 | 3: 54185471 |
| 14632 | 3: 54186364 | 14633 | 3: 54188660 | 14634 | 3: 54201062 | 14638 | 3: 54367531 | 14649 | 3: 55014493 |
| 14650 | 3: 55022327 | 14708 | 3: 56670745 | 14714 | 3: 56924360 | 14716 | 3: 56934727 | 14717 | 3: 56936672 |
| 14719 | 3: 56953053 | 14721 | 3: 56999586 | 14723 | 3: 57032150 | 14727 | 3: 57245572 | 14745 | 3: 57799143 |
| 14749 | 3: 57886444 | 14757 | 3: 58161126 | 14767 | 3: 58455621 | 14770 | 3: 58489145 | 14772 | 3: 58507559 |
| 14776 | 3: 58545248 | 14787 | 3: 58913251 | 14791 | 3: 59017336 | 14795 | 3: 59162716 | 14796 | 3: 59207859 |
| 14816 | 3: 59758400 | 14821 | 3: 60026204 | 14826 | 3: 60180066 | 14831 | 3: 60261618 | 14849 | 3: 60625549 |
| 14858 | 3: 60737754 | 14867 | 3: 60914592 | 14869 | 3: 60962012 | 14888 | 3: 61496668 | 14893 | 3: 61744158 |
| 14896 | 3: 61858795 | 14904 | 3: 62095195 | 14910 | 3: 62278399 | 14914 | 3: 62359529 | 14933 | 3: 62854439 |
| 14945 | 3: 63290027 | 14972 | 3: 63993039 | 14976 | 3: 64069515 | 14991 | 3: 64651171 | 14995 | 3: 64790813 |
| 15006 | 3: 65095548 | 15008 | 3: 65157057 | 15028 | 3: 65566921 | 15032 | 3: 65663032 | 15057 | 3: 66345116 |
| 15058 | 3: 66351139 | 15061 | 3: 66398657 | 15066 | 3: 66502134 | 15070 | 3: 66529229 | 15078 | 3: 66637168 |
| 15083 | 3: 66838529 | 15089 | 3: 66948480 | 15091 | 3: 66974396 | 15092 | 3: 66999361 | 15093 | 3: 67031006 |
| 15095 | 3: 67168089 | 15102 | 3: 67318712 | 15109 | 3: 67473906 | 15116 | 3: 67670465 | 15134 | 3: 68047509 |
| 15140 | 3: 68228132 | 15141 | 3: 68228231 | 15142 | 3: 68276086 | 15143 | 3: 68302197 | 15145 | 3: 68316834 |
| 15149 | 3: 68422911 | 15151 | 3: 68443638 | 15156 | 3: 68551519 | 15158 | 3: 68594137 | 15165 | 3: 68727455 |
| 15166 | 3: 68788461 | 15173 | 3: 68973246 | 15174 | 3: 69004319 | 15177 | 3: 69066161 | 15184 | 3: 69392637 |
| 15226 | 3: 70427777 | 15229 | 3: 70602082 | 15230 | 3: 70652398 | 15255 | 3: 71704029 | 15272 | 3: 72307570 |
| 15273 | 3: 72307597 | 15294 | 3: 73250579 | 15305 | 3: 73571608 | 15309 | 3: 73698701 | 15310 | 3: 73698721 |
| 15316 | 3: 73904881 | 15317 | 3: 73909382 | 15324 | 3: 74202751 | 15327 | 3: 74264677 | 15345 | 3: 74829928 |
| 15349 | 3: 74963727 | 15355 | 3: 75071726 | 15364 | 3: 75370499 | 15370 | 3: 75506540 | 15374 | 3: 75649317 |
| 15375 | 3: 75649320 | 15376 | 3: 75649336 | 15377 | 3: 75649383 | 15398 | 3: 76298830 | 15406 | 3: 76468869 |
| 15415 | 3: 76956223 | 15417 | 3: 77030221 | 15423 | 3: 77162998 | 15425 | 3: 77178882 | 15434 | 3: 77402844 |
| 15435 | 3: 77409935 | 15438 | 3: 77489908 | 15441 | 3: 77571521 | 15449 | 3: 77850286 | 15459 | 3: 78097452 |
| 15465 | 3: 78290774 | 15468 | 3: 78420363 | 15473 | 3: 78568977 | 15474 | 3: 78569227 | 15475 | 3: 78569835 |
| 15478 | 3: 78642264 | 15479 | 3: 78662366 | 15481 | 3: 78785400 | 15489 | 3: 79003182 | 15491 | 3: 79007539 |
| 15497 | 3: 79179242 | 15510 | 3: 79464743 | 15523 | 3: 79956010 | 15525 | 3: 79973859 | 15531 | 3: 80163495 |
| 15537 | 3: 80491657 | 15540 | 3: 80549623 | 15548 | 3: 80733840 | 15556 | 3: 80952119 | 15557 | 3: 80954660 |
| 15558 | 3: 80954761 | 15560 | 3: 80999275 | 15566 | 3: 81103725 | 15578 | 3: 81314640 | 15580 | 3: 81343738 |
| 15582 | 3: 81436844 | 15584 | 3: 81464927 | 15597 | 3: 81749835 | 15613 | 3: 82402375 | 15616 | 3: 82454574 |
| 15620 | 3: 82503792 | 15655 | 3: 83636824 | 15658 | 3: 83652637 | 15673 | 3: 84036094 | 15680 | 3: 84195762 |
| 15694 | 3: 84534786 | 15695 | 3: 84550534 | 15697 | 3: 84560190 | 15726 | 3: 84721616 | 15747 | 3: 85077748 |
| 15751 | 3: 85284944 | 15755 | 3: 85429614 | 15756 | 3: 85429668 | 15757 | 3: 85429696 | 15775 | 3: 85848368 |
| 15783 | 3: 85978048 | 15791 | 3: 86156348 | 15797 | 3: 86290419 | 15800 | 3: 86312689 | 15801 | 3: 86312736 |
| 15805 | 3: 86366796 | 15811 | 3: 86584920 | 15815 | 3: 86652496 | 15827 | 3: 86872323 | 15840 | 3: 87228581 |
| 15843 | 3: 87295571 | 15847 | 3: 87349602 | 15848 | 3: 87379701 | 15929 | 3: 88997759 | 15930 | 3: 89044393 |
| 15932 | 3: 89092242 | 15936 | 3: 89255790 | 15957 | 3: 89639867 | 15963 | 3: 89717766 | 15964 | 3: 89722428 |
| 15966 | 3: 89773983 | 15968 | 3: 89792849 | 15971 | 3: 89858003 | 15972 | 3: 89858025 | 15973 | 3: 89861756 |
| 15974 | 3: 89888858 | 15979 | 3: 89947137 | 15983 | 3: 90125909 | 15987 | 3: 90218397 | 15996 | 3: 90458410 |
| 15998 | 3: 90490960 | 16012 | 3: 90774079 | 16022 | 3: 90916704 | 16028 | 3: 91085209 | 16032 | 3: 91177765 |
| 16038 | 3: 91342742 | 16039 | 3: 91349417 | 16047 | 3: 91577320 | 16050 | 3: 91662702 | 16056 | 3: 91779733 |
| 16057 | 3: 91821560 | 16058 | 3: 91850874 | 16061 | 3: 91944104 | 16064 | 3: 92012709 | 16066 | 3: 92056509 |
| 16072 | 3: 92226650 | 16083 | 3: 92532036 | 16092 | 3: 92747667 | 16093 | 3: 92757686 | 16096 | 3: 92813097 |
| 16103 | 3: 92955459 | 16105 | 3: 92979991 | 16112 | 3: 93070031 | 16115 | 3: 93079352 | 16117 | 3: 93087724 |
| 16123 | 3: 93219307 | 16124 | 3: 93219814 | 16125 | 3: 93220543 | 16126 | 3: 93226524 | 16127 | 3: 93235948 |
| 16128 | 3: 93314047 | 16130 | 3: 93345899 | 16143 | 3: 93782247 | 16144 | 3: 93782331 | 16145 | 3: 93782436 |
| 16159 | 3: 94060442 | 16165 | 3: 94226523 | 16178 | 3: 94716721 | 16180 | 3: 94726765 | 16183 | 3: 94823429 |
| 16184 | 3: 94866313 | 16186 | 3: 94911090 | 16188 | 3: 94976568 | 16190 | 3: 95036766 | 16230 | 3: 96202292 |
| 16238 | 3: 96579309 | 16242 | 3: 96636004 | 16247 | 3: 96785820 | 16248 | 3: 96830575 | 16254 | 3: 96975581 |
| 16260 | 3: 97123954 | 16269 | 3: 97365473 | 16274 | 3: 97457441 | 16275 | 3: 97473661 | 16278 | 3: 97526789 |
| 16290 | 3: 97708909 | 16301 | 3: 97930159 | 16305 | 3: 98116083 | 16309 | 3: 98295744 | 16313 | 3: 98349294 |
| 16317 | 3: 98403571 | 16321 | 3: 98561508 | 16335 | 3: 98923450 | 16337 | 3: 98978448 | 16338 | 3: 98978458 |
| 16348 | 3: 99342353 | 16350 | 3: 99349479 | 16351 | 3: 99356345 | 16352 | 3: 99360354 | 16353 | 3: 99377234 |
| 16358 | 3: 99519922 | 16368 | 3: 99751257 | 16369 | 3: 99765693 | 16370 | 3: 99771380 | 16371 | 3: 99805386 |
| 16372 | 3: 99811235 | 16373 | 3: 99811520 | 16374 | 3: 99818061 | 16375 | 3: 99831451 | 16376 | 3: 99840460 |
| 16377 | 3: 99840480 | 16378 | 3: 99884150 | 16381 | 3: 99928886 | 16383 | 3: 99953595 | 16387 | 3: 100006117 |
| 16388 | 3: 100006731 | 16391 | 3: 100135156 | 16393 | 3: 100148664 | 16403 | 3: 100386736 | 16405 | 3: 100413711 |
| 16406 | 3: 100417680 | 16407 | 3: 100429883 | 16410 | 3: 100503760 | 16411 | 3: 100519527 | 16412 | 3: 100527924 |
| 16413 | 3: 100532091 | 16415 | 3: 100578191 | 16417 | 3: 100604376 | 16418 | 3: 100616906 | 16420 | 3: 100649238 |
| 16421 | 3: 100655322 | 16422 | 3: 100660031 | 16425 | 3: 100706971 | 16426 | 3: 100710303 | 16435 | 3: 100839974 |
| 16440 | 3: 101045558 | 16449 | 3: 101184420 | 16456 | 3: 101301924 | 16457 | 3: 101328497 | 16462 | 3: 101381082 |
| 16496 | 3: 102252547 | 16500 | 3: 102323975 | 16502 | 3: 102346918 | 16504 | 3: 102360619 | 16505 | 3: 102430768 |
| 16506 | 3: 102432444 | 16515 | 3: 102664478 | 16516 | 3: 102684885 | 16517 | 3: 102684933 | 16518 | 3: 102684985 |
| 16520 | 3: 102736091 | 16521 | 3: 102753266 | 16524 | 3: 102804450 | 16529 | 3: 102845961 | 16530 | 3: 102846036 |
| 16531 | 3: 102853282 | 16536 | 3: 102881866 | 16555 | 3: 103364021 | 16560 | 3: 103438363 | 16564 | 3: 103569295 |
| 16566 | 3: 103617298 | 16567 | 3: 103655132 | 16569 | 3: 103692819 | 16571 | 3: 103732748 | 16573 | 3: 103756143 |
| 16578 | 3: 103836979 | 16581 | 3: 103923977 | 16583 | 3: 104007365 | 16584 | 3: 104022881 | 16590 | 3: 104089157 |
| 16598 | 3: 104333611 | 16610 | 3: 104577770 | 16612 | 3: 104588926 | 16614 | 3: 104626150 | 16616 | 3: 104664666 |
| 16618 | 3: 104700803 | 16621 | 3: 104722133 | 16622 | 3: 104733085 | 16624 | 3: 104818821 | 16629 | 3: 104879046 |
| 16641 | 3: 105195217 | 16642 | 3: 105195508 | 16644 | 3: 105197594 | 16645 | 3: 105207363 | 16648 | 3: 105223366 |
| 16649 | 3: 105224129 | 16650 | 3: 105224197 | 16656 | 3: 105344373 | 16657 | 3: 105346169 | 16658 | 3: 105386965 |
| 16659 | 3: 105397156 | 16660 | 3: 105399284 | 16661 | 3: 105401478 | 16664 | 3: 105416679 | 16665 | 3: 105419956 |
| 16666 | 3: 105420955 | 16668 | 3: 105522467 | 16669 | 3: 105528154 | 16670 | 3: 105530865 | 16671 | 3: 105550247 |
| 16672 | 3: 105553161 | 16673 | 3: 105557267 | 16675 | 3: 105587522 | 16676 | 3: 105587615 | 16677 | 3: 105588435 |
| 16678 | 3: 105589110 | 16679 | 3: 105589417 | 16680 | 3: 105589499 | 16681 | 3: 105589515 | 16682 | 3: 105589567 |
| 16683 | 3: 105589840 | 16686 | 3: 105615307 | 16687 | 3: 105623790 | 16688 | 3: 105633378 | 16689 | 3: 105633804 |
| 16691 | 3: 105644182 | 16692 | 3: 105654662 | 16693 | 3: 105674940 | 16696 | 3: 105745106 | 16701 | 3: 105803674 |
| 16702 | 3: 105805027 | 16705 | 3: 105897358 | 16706 | 3: 105897511 | 16707 | 3: 105897845 | 16708 | 3: 105897970 |
| 16709 | 3: 105908966 | 16710 | 3: 105909058 | 16712 | 3: 105935499 | 16713 | 3: 105956333 | 16717 | 3: 106020186 |
| 16724 | 3: 106135737 | 16731 | 3: 106244085 | 16735 | 3: 106337682 | 16745 | 3: 106561725 | 16746 | 3: 106561875 |
| 16747 | 3: 106585197 | 16748 | 3: 106591729 | 16754 | 3: 106655656 | 16755 | 3: 106668839 | 16765 | 3: 106845702 |
| 16771 | 3: 107103522 | 16783 | 3: 107354102 | 16784 | 3: 107375067 | 16785 | 3: 107375088 | 16786 | 3: 107375142 |
| 16789 | 3: 107445270 | 16791 | 3: 107477138 | 16800 | 3: 107604142 | 16807 | 3: 107789881 | 16817 | 3: 107937893 |
| 16819 | 3: 107940715 | 16830 | 3: 108203613 | 16831 | 3: 108224103 | 16833 | 3: 108305450 | 16835 | 3: 108345650 |
| 16850 | 3: 108755292 | 16851 | 3: 108756407 | 16852 | 3: 108760658 | 16853 | 3: 108761540 | 16854 | 3: 108763978 |
| 16855 | 3: 108764835 | 16856 | 3: 108773793 | 16857 | 3: 108777623 | 16858 | 3: 108778270 | 16860 | 3: 108788102 |
| 16861 | 3: 108817854 | 16862 | 3: 108824614 | 16864 | 3: 108842377 | 16885 | 3: 109426329 | 16888 | 3: 109430792 |
| 16889 | 3: 109430841 | 16899 | 3: 109606532 | 16900 | 3: 109656896 | 16916 | 3: 109969045 | 6920 | 3: 110020722 |
| 16921 | 3: 110026915 | 16923 | 3: 110036182 | 16927 | 3: 110153404 | 16946 | 3: 110780122 | 16947 | 3: 110781851 |
| 16948 | 3: 110783875 | 16949 | 3: 110784912 | 16950 | 3: 110786097 | 16954 | 3: 110849640 | 16978 | 3: 111418313 |
| 16986 | 3: 111526183 | 16989 | 3: 111580049 | 17003 | 3: 111889624 | 17019 | 3: 112192839 | 17025 | 3: 112323001 |
| 17026 | 3: 112345897 | 17032 | 3: 112397006 | 17046 | 3: 112761486 | 17047 | 3: 112761940 | 17050 | 3: 112794864 |
| 17058 | 3: 113036203 | 17062 | 3: 113127829 | 17063 | 3: 113131422 | 17064 | 3: 113146141 | 17072 | 3: 113331205 |
| 17073 | 3: 113331220 | 17074 | 3: 113337934 | 17075 | 3: 113341660 | 17078 | 3: 113407286 | 17081 | 3: 113453006 |
| 17083 | 3: 113518551 | 17084 | 3: 113518636 | 17085 | 3: 113524422 | 17089 | 3: 113563964 | 17090 | 3: 113563979 |
| 17091 | 3: 113563997 | 17092 | 3: 113564006 | 17093 | 3: 113564076 | 17094 | 3: 113564096 | 17095 | 3: 113564107 |
| 17096 | 3: 113585771 | 17097 | 3: 113585795 | 17098 | 3: 113585799 | 17100 | 3: 113586101 | 17101 | 3: 113586251 |
| 17102 | 3: 113590170 | 17120 | 3: 114115462 | 17126 | 3: 114165101 | 17137 | 3: 114384157 | 17141 | 3: 114446007 |
| 17144 | 3: 114496084 | 17146 | 3: 114508185 | 17149 | 3: 114543461 | 17150 | 3: 114548928 | 17152 | 3: 114594768 |
| 17156 | 3: 114618765 | 17160 | 3: 114704691 | 17164 | 3: 114748408 | 17175 | 3: 114802567 | 17185 | 3: 114881902 |
| 17190 | 3: 115000401 | 17193 | 3: 115052234 | 17199 | 3: 115107550 | 17200 | 3: 115120882 | 17208 | 3: 115363288 |
| 17210 | 3: 115374146 | 17213 | 3: 115397893 | 17222 | 3: 115484538 | 17225 | 3: 115543657 | 17227 | 3: 115544750 |
| 17231 | 3: 115602232 | 17232 | 3: 115602518 | 17237 | 3: 115733885 | 17238 | 3: 115814948 | 17243 | 3: 115936238 |
| 17246 | 3: 115967688 | 17262 | 3: 116221838 | 17264 | 3: 116272992 | 17270 | 3: 116347067 | 17282 | 3: 116562510 |
| 17291 | 3: 116772922 | 17299 | 3: 116861866 | 17310 | 3: 116928313 | 17314 | 3: 116981402 | 17315 | 3: 116981583 |
| 17316 | 3: 116981638 | 17320 | 3: 117000919 | 17323 | 3: 117031812 | 17324 | 3: 117049753 | 17326 | 3: 117070587 |
| 17330 | 3: 117123078 | 17331 | 3: 117126904 | 17334 | 3: 117149901 | 17337 | 3: 117209899 | 17338 | 3: 117209960 |
| 17341 | 3: 117278310 | 17342 | 3: 117283114 | 17344 | 3: 117317452 | 17345 | 3: 117325008 | 17353 | 3: 117578080 |
| 17360 | 3: 117649528 | 17361 | 3: 117649537 | 17362 | 3: 117662078 | 17365 | 3: 117673624 | 17366 | 3: 117696207 |
| 17370 | 3: 117709813 | 17394 | 3: 118128778 | 17401 | 3: 118223118 | 17403 | 3: 118288926 | 17404 | 3: 118289376 |
| 17405 | 3: 118289408 | 17406 | 3: 118289679 | 17411 | 3: 118387538 | 17420 | 3: 118548276 | 17426 | 3: 118615619 |
| 17436 | 3: 118755947 | 17440 | 3: 118806132 | 17449 | 3: 118883045 | 17457 | 3: 118964562 | 17461 | 3: 119094347 |
| 17467 | 3: 119427638 | 17472 | 3: 119648530 | 17475 | 3: 119673122 | 17476 | 3: 119709097 | 17481 | 3: 119787932 |
| 17486 | 3: 119868121 | 17499 | 3: 120268455 | 17502 | 3: 120306744 | 17503 | 3: 120306964 | 17510 | 3: 120576521 |
| 17515 | 3: 120695918 | 17522 | 3: 120790650 | 17553 | 4: 1446328 | 17648 | 4: 4173794 | 17649 | 4: 4184943 |
| 17663 | 4: 4533982 | 17666 | 4: 4582744 | 17674 | 4: 4681609 | 17675 | 4: 4681672 | 17679 | 4: 4733530 |
| 17686 | 4: 4818261 | 17691 | 4: 4871624 | 17692 | 4: 4931247 | 17693 | 4: 4943634 | 17701 | 4: 5077162 |
| 17703 | 4: 5134591 | 17708 | 4: 5202035 | 17714 | 4: 5319445 | 17715 | 4: 5319859 | 17721 | 4: 5377091 |
| 17722 | 4: 5395548 | 17724 | 4: 5422821 | 17725 | 4: 5437743 | 17726 | 4: 5441832 | 17756 | 4: 6207303 |
| 17787 | 4: 7009102 | 17788 | 4: 7009111 | 17792 | 4: 7073207 | 17793 | 4: 7106157 | 17799 | 4: 7247371 |
| 17808 | 4: 7354530 | 17814 | 4: 7445669 | 17820 | 4: 7632416 | 17821 | 4: 7658763 | 17822 | 4: 7658787 |
| 17823 | 4: 7658831 | 17824 | 4: 7658948 | 17825 | 4: 7658962 | 17827 | 4: 7697381 | 17835 | 4: 7933911 |
| 17836 | 4: 7945782 | 17837 | 4: 7996659 | 17840 | 4: 8046313 | 17844 | 4: 8179768 | 17847 | 4: 8246510 |
| 17850 | 4: 8272646 | 17853 | 4: 8420098 | 17854 | 4: 8435396 | 17856 | 4: 8445348 | 17859 | 4: 8506844 |
| 17883 | 4: 9151646 | 17891 | 4: 9271969 | 17893 | 4: 9323578 | 17896 | 4: 9385962 | 17898 | 4: 9407447 |
| 17900 | 4: 9444470 | 17901 | 4: 9474683 | 17902 | 4: 9477621 | 17904 | 4: 9485074 | 17905 | 4: 9486301 |
| 17906 | 4: 9488068 | 17907 | 4: 9490132 | 17908 | 4: 9490936 | 17909 | 4: 9492909 | 17920 | 4: 9817295 |
| 17949 | 4: 10163330 | 17953 | 4: 10231259 | 17963 | 4: 10387935 | 17973 | 4: 10679109 | 17996 | 4: 11190947 |
| 17997 | 4: 11191071 | 18000 | 4: 11265836 | 18001 | 4: 11269876 | 18002 | 4: 11271703 | 18007 | 4: 11332982 |
| 18008 | 4: 11339823 | 18009 | 4: 11344256 | 18010 | 4: 11344907 | 18029 | 4: 11936615 | 18050 | 4: 12576634 |
| 18052 | 4: 12673015 | 18061 | 4: 12802609 | 18063 | 4: 12826295 | 18066 | 4: 12860940 | 18072 | 4: 13010035 |
| 18091 | 4: 13579464 | 18092 | 4: 13579504 | 18093 | 4: 13579532 | 18098 | 4: 13707039 | 18102 | 4: 13801041 |
| 18106 | 4: 14002326 | 18110 | 4: 14035164 | 18111 | 4: 14061507 | 18112 | 4: 14079747 | 18148 | 4: 15337479 |
| 18153 | 4: 15469076 | 18154 | 4: 15470050 | 18160 | 4: 15586640 | 18182 | 4: 16245146 | 18200 | 4: 16761268 |
| 18210 | 4: 17040967 | 18221 | 4: 17237895 | 18235 | 4: 17572154 | 18236 | 4: 17572159 | 18237 | 4: 17572202 |
| 18238 | 4: 17572213 | 18256 | 4: 18084841 | 18272 | 4: 18585854 | 18277 | 4: 18655237 | 18279 | 4: 18662663 |
| 18280 | 4: 18669066 | 18282 | 4: 18685097 | 18284 | 4: 18697683 | 18286 | 4: 18705074 | 18287 | 4: 18707492 |
| 18293 | 4: 18889879 | 18325 | 4: 19569259 | 18363 | 4: 19999023 | 18368 | 4: 20111420 | 18373 | 4: 20255515 |
| 18379 | 4: 20329251 | 18380 | 4: 20329405 | 18397 | 4: 20796684 | 18398 | 4: 20802780 | 18399 | 4: 20804901 |
| 18419 | 4: 21160009 | 18420 | 4: 21160045 | 18422 | 4: 21189572 | 18424 | 4: 21207059 | 18425 | 4: 21213102 |
| 18460 | 4: 22190378 | 18463 | 4: 22352000 | 18469 | 4: 22526571 | 18488 | 4: 22913622 | 18498 | 4: 23160284 |
| 18510 | 4: 23595303 | 18521 | 4: 23872526 | 18523 | 4: 23891166 | 18526 | 4: 23927655 | 18527 | 4: 23946714 |
| 18536 | 4: 24333008 | 18549 | 4: 24650631 | 18554 | 4: 24810069 | 18557 | 4: 24885290 | 18565 | 4: 25097829 |
| 18566 | 4: 25109810 | 18567 | 4: 25127084 | 18583 | 4: 25647682 | 18587 | 4: 25742989 | 18590 | 4: 25907759 |
| 18591 | 4: 25907762 | 18594 | 4: 25924624 | 18609 | 4: 26257023 | 18621 | 4: 26648480 | 18627 | 4: 26786277 |
| 18641 | 4: 27097020 | 18642 | 4: 27127515 | 18654 | 4: 27476944 | 18662 | 4: 27629208 | 18666 | 4: 27698262 |
| 18681 | 4: 28186796 | 18682 | 4: 28217559 | 18686 | 4: 28338304 | 18690 | 4: 28410444 | 18691 | 4: 28434228 |
| 18703 | 4: 28719533 | 18704 | 4: 28738300 | 18712 | 4: 28975908 | 18714 | 4: 29080932 | 18716 | 4: 29142901 |
| 18745 | 4: 30297344 | 18752 | 4: 30430428 | 18753 | 4: 30430450 | 18754 | 4: 30430542 | 18755 | 4: 30432018 |
| 18757 | 4: 30501662 | 18784 | 4: 31141033 | 18790 | 4: 31306787 | 18795 | 4: 31471170 | 18797 | 4: 31535187 |
| 18801 | 4: 31680539 | 18809 | 4: 31817533 | 18810 | 4: 31854356 | 18814 | 4: 31949743 | 18854 | 4: 33211684 |
| 18858 | 4: 33325825 | 18869 | 4: 33588518 | 18873 | 4: 33639372 | 18874 | 4: 33652107 | 18876 | 4: 33739841 |
| 18878 | 4: 33761684 | 18880 | 4: 33789343 | 18887 | 4: 33926324 | 18894 | 4: 34109934 | 18913 | 4: 34771597 |
| 18921 | 4: 35053751 | 18923 | 4: 35162460 | 18934 | 4: 35468668 | 18964 | 4: 36420563 | 18970 | 4: 36597277 |
| 18971 | 4: 36657445 | 18981 | 4: 36944816 | 18996 | 4: 37260138 | 19008 | 4: 37691142 | 19021 | 4: 38080777 |
| 19023 | 4: 38088154 | 19027 | 4: 38196269 | 19029 | 4: 38253124 | 19034 | 4: 38381571 | 19048 | 4: 38785910 |
| 19058 | 4: 39096980 | 19077 | 4: 39839146 | 19079 | 4: 39869029 | 19096 | 4: 40085698 | 19097 | 4: 40100668 |
| 19111 | 4: 40502117 | 19112 | 4: 40545187 | 19120 | 4: 40660943 | 19122 | 4: 40678229 | 19128 | 4: 40850556 |
| 19131 | 4: 40925102 | 19136 | 4: 40978581 | 19137 | 4: 40990306 | 19138 | 4: 40990370 | 19139 | 4: 40990413 |
| 19140 | 4: 40994417 | 19143 | 4: 41012545 | 19144 | 4: 41012589 | 19145 | 4: 41012593 | 19155 | 4: 41242597 |
| 19171 | 4: 42001110 | 19176 | 4: 42079882 | 19179 | 4: 42269505 | 19186 | 4: 42478244 | 19198 | 4: 42862322 |
| 19217 | 4: 43233221 | 19218 | 4: 43233239 | 19219 | 4: 43233296 | 19220 | 4: 43233323 | 19221 | 4: 43233412 |
| 19223 | 4: 43330695 | 19234 | 4: 43684052 | 19242 | 4: 43934934 | 19244 | 4: 43942237 | 19248 | 4: 44063960 |
| 19253 | 4: 44178421 | 19256 | 4: 44256983 | 19257 | 4: 44268015 | 19260 | 4: 44305806 | 19268 | 4: 44547450 |
| 19282 | 4: 45052002 | 19298 | 4: 45602329 | 19311 | 4: 46002303 | 19315 | 4: 46099926 | 19317 | 4: 46129459 |
| 19329 | 4: 46600207 | 19340 | 4: 46830510 | 19345 | 4: 46921985 | 19346 | 4: 46925441 | 19347 | 4: 46925682 |
| 19348 | 4: 46943050 | 19349 | 4: 46943107 | 19350 | 4: 46943131 | 19363 | 4: 47258425 | 19379 | 4: 47479517 |
| 19383 | 4: 47511989 | 19386 | 4: 47569946 | 19397 | 4: 47904922 | 19399 | 4: 48028783 | 19403 | 4: 48241131 |
| 19420 | 4: 48780844 | 19426 | 4: 49017869 | 19428 | 4: 49022872 | 19430 | 4: 49035315 | 19431 | 4: 49035320 |
| 19432 | 4: 49035381 | 19433 | 4: 49035395 | 19434 | 4: 49035467 | 19435 | 4: 49037953 | 19437 | 4: 49045621 |
| 19438 | 4: 49045631 | 19439 | 4: 49045737 | 19440 | 4: 49045753 | 19441 | 4: 49045756 | 19442 | 4: 49045852 |
| 19444 | 4: 49061063 | 19445 | 4: 49062116 | 19447 | 4: 49107209 | 19452 | 4: 49181169 | 19456 | 4: 49269697 |
| 19457 | 4: 49284053 | 19467 | 4: 49523230 | 19478 | 4: 49842196 | 19482 | 4: 49964915 | 19486 | 4: 50057099 |
| 19489 | 4: 50248085 | 19490 | 4: 50345064 | 19496 | 4: 50538062 | 19515 | 4: 51250626 | 19517 | 4: 51303879 |
| 19526 | 4: 51543766 | 19532 | 4: 51713354 | 19581 | 4: 53246820 | 19594 | 4: 53516423 | 19599 | 4: 53675011 |
| 19614 | 4: 54062446 | 19622 | 4: 54498204 | 19634 | 4: 54973210 | 19638 | 4: 55064494 | 19643 | 4: 55358812 |
| 19646 | 4: 55397907 | 19658 | 4: 55841154 | 19659 | 4: 55864590 | 19660 | 4: 55873977 | 19663 | 4: 55914722 |
| 19665 | 4: 55965169 | 19671 | 4: 56042508 | 19674 | 4: 56091771 | 19683 | 4: 56311977 | 19689 | 4: 56597023 |
| 19691 | 4: 56614952 | 19696 | 4: 56795183 | 19706 | 4: 57053471 | 19711 | 4: 57121533 | 19713 | 4: 57264096 |
| 19722 | 4: 57443352 | 19726 | 4: 57549751 | 19727 | 4: 57552140 | 19730 | 4: 57682773 | 19735 | 4: 57810438 |
| 19764 | 4: 58952251 | 19771 | 4: 59151344 | 19778 | 4: 59234606 | 19796 | 4: 59761295 | 19798 | 4: 59797184 |
| 19799 | 4: 59797205 | 19817 | 4: 60407425 | 19823 | 4: 60610913 | 19829 | 4: 60822961 | 19835 | 4: 61004115 |
| 19843 | 4: 61150481 | 19844 | 4: 61176028 | 19845 | 4: 61188646 | 19868 | 4: 61850527 | 19876 | 4: 62043721 |
| 19886 | 4: 62197348 | 19901 | 4: 62758226 | 19902 | 4: 62762359 | 19903 | 4: 62769531 | 19910 | 4: 62994404 |
| 19911 | 4: 63001098 | 19919 | 4: 63185750 | 19922 | 4: 63289063 | 19926 | 4: 63398159 | 19932 | 4: 63595012 |
| 19945 | 4: 63872067 | 19946 | 4: 63887485 | 19948 | 4: 63898910 | 19949 | 4: 63913879 | 19957 | 4: 64206739 |
| 19966 | 4: 64550273 | 19968 | 4: 64596464 | 19969 | 4: 64598653 | 19970 | 4: 64633068 | 19997 | 4: 65579692 |
| 19998 | 4: 65585572 | 20000 | 4: 65586522 | 20004 | 4: 65662118 | 20005 | 4: 65663680 | 20006 | 4: 65663924 |
| 20011 | 4: 65757109 | 20016 | 4: 65871790 | 20018 | 4: 65887212 | 20020 | 4: 65935466 | 20026 | 4: 66031691 |
| 20030 | 4: 66126342 | 20032 | 4: 66167545 | 20040 | 4: 66351372 | 20044 | 4: 66424351 | 20058 | 4: 66948425 |
| 20064 | 4: 67093419 | 20079 | 4: 67478622 | 20089 | 4: 67825161 | 20093 | 4: 67886242 | 20098 | 4: 67941747 |
| 20103 | 4: 68103259 | 20106 | 4: 68130974 | 20112 | 4: 68281786 | 20118 | 4: 68525805 | 20138 | 4: 69316258 |
| 20146 | 4: 69461895 | 20150 | 4: 69590761 | 20153 | 4: 69624135 | 20162 | 4: 69834879 | 20168 | 4: 69953830 |
| 20177 | 4: 70190883 | 20179 | 4: 70206948 | 20181 | 4: 70239423 | 20184 | 4: 70315106 | 20197 | 4: 70629516 |
| 20200 | 4: 70650674 | 20207 | 4: 70942839 | 20208 | 4: 70955859 | 20210 | 4: 71014142 | 20212 | 4: 71044933 |
| 20241 | 4: 71963805 | 20242 | 4: 71995369 | 20247 | 4: 72082867 | 20250 | 4: 72155086 | 20265 | 4: 72831364 |
| 20266 | 4: 72831400 | 20278 | 4: 73141876 | 20280 | 4: 73230353 | 20281 | 4: 73230433 | 20282 | 4: 73230506 |
| 20287 | 4: 73356120 | 20295 | 4: 73677765 | 20320 | 4: 74355629 | 20326 | 4: 74574092 | 20327 | 4: 74575187 |
| 20328 | 4: 74575293 | 20332 | 4: 74690642 | 20336 | 4: 74750397 | 20337 | 4: 74775088 | 20339 | 4: 74788299 |
| 20347 | 4: 74921800 | 20348 | 4: 74931461 | 20349 | 4: 74937132 | 20351 | 4: 74962440 | 20355 | 4: 75035303 |
| 20360 | 4: 75176025 | 20375 | 4: 75585274 | 20376 | 4: 75596693 | 20395 | 4: 75964360 | 20410 | 4: 76300291 |
| 20411 | 4: 76303426 | 20427 | 4: 76654098 | 20428 | 4: 76665449 | 20431 | 4: 76826893 | 20434 | 4: 77005397 |
| 20438 | 4: 77038646 | 20439 | 4: 77114794 | 20440 | 4: 77131669 | 20441 | 4: 77141818 | 20443 | 4: 77158468 |
| 20446 | 4: 77222263 | 20447 | 4: 77232107 | 20448 | 4: 77235523 | 20449 | 4: 77272270 | 20453 | 4: 77462163 |
| 20459 | 4: 77550845 | 20460 | 4: 77550847 | 20461 | 4: 77550961 | 20463 | 4: 77552548 | 20464 | 4: 77552605 |
| 20465 | 4: 77552647 | 20466 | 4: 77562640 | 20467 | 4: 77564937 | 20468 | 4: 77565007 | 20469 | 4: 77565031 |
| 20470 | 4: 77612846 | 20474 | 4: 77703279 | 20484 | 4: 78046476 | 20493 | 4: 78275533 | 20500 | 4: 78501007 |
| 20516 | 4: 79081070 | 20532 | 4: 79524078 | 20534 | 4: 79581997 | 20535 | 4: 79582037 | 20570 | 4: 80210945 |
| 20571 | 4: 80211747 | 20575 | 4: 80312765 | 20577 | 4: 80342472 | 20578 | 4: 80347612 | 20603 | 4: 81058244 |
| 20606 | 4: 81115882 | 20608 | 4: 81125829 | 20612 | 4: 81203512 | 20613 | 4: 81222120 | 20615 | 4: 81291786 |
| 20624 | 4: 81541207 | 20646 | 4: 82094311 | 20656 | 4: 82418721 | 20664 | 4: 82649849 | 20665 | 4: 82649930 |
| 20668 | 4: 82720591 | 20669 | 4: 82720596 | 20670 | 4: 82720610 | 20671 | 4: 82720657 | 20672 | 4: 82720686 |
| 20673 | 4: 82720715 | 20679 | 4: 82824766 | 20696 | 4: 83387242 | 20697 | 4: 83420381 | 20699 | 4: 83506707 |
| 20700 | 4: 83540447 | 20701 | 4: 83605440 | 20725 | 4: 84374337 | 20748 | 4: 84998314 | 20767 | 4: 85529127 |
| 20769 | 4: 85585852 | 20770 | 4: 85589724 | 20771 | 4: 85609908 | 20783 | 4: 85887734 | 20784 | 4: 85902484 |
| 20791 | 4: 86035429 | 20794 | 4: 86107048 | 20805 | 4: 86429759 | 20807 | 4: 86434813 | 20808 | 4: 86453759 |
| 20812 | 4: 86588536 | 20827 | 4: 86869600 | 20831 | 4: 87015587 | 20832 | 4: 87025872 | 20833 | 4: 87026389 |
| 20847 | 4: 87544345 | 20861 | 4: 87862791 | 20869 | 4: 87966655 | 20870 | 4: 87988315 | 20871 | 4: 88003339 |
| 20874 | 4: 88190387 | 20876 | 4: 88214757 | 20877 | 4: 88230965 | 20878 | 4: 88267363 | 20883 | 4: 88482339 |
| 20916 | 4: 89540510 | 20919 | 4: 89600401 | 20920 | 4: 89600435 | 20921 | 4: 89600533 | 20925 | 4: 89717462 |
| 20926 | 4: 89720129 | 20997 | 4: 91292909 | 21000 | 4: 91358381 | 21011 | 4: 91589372 | 21017 | 4: 91792117 |
| 21021 | 4: 91837317 | 21032 | 4: 92071091 | 21034 | 4: 92150036 | 21036 | 4: 92281567 | 21038 | 4: 92292551 |
| 21039 | 4: 92298261 | 21040 | 4: 92299550 | 21041 | 4: 92307140 | 21042 | 4: 92315009 | 21044 | 4: 92351942 |
| 21049 | 4: 92444591 | 21058 | 4: 92540924 | 21062 | 4: 92634658 | 21065 | 4: 92695366 | 21072 | 4: 92893919 |
| 21083 | 4: 93143125 | 21086 | 4: 93270718 | 21088 | 4: 93325674 | 21091 | 4: 93338554 | 21092 | 4: 93343119 |
| 21093 | 4: 93349924 | 21094 | 4: 93350879 | 21095 | 4: 93352485 | 21096 | 4: 93361239 | 21097 | 4: 93361477 |
| 21098 | 4: 93362689 | 21099 | 4: 93368708 | 21100 | 4: 93373657 | 21101 | 4: 93376458 | 21102 | 4: 93388339 |
| 21104 | 4: 93445593 | 21107 | 4: 93470782 | 21114 | 4: 93581039 | 21121 | 4: 93756393 | 21125 | 4: 93956921 |
| 21138 | 4: 94448413 | 21147 | 4: 94698872 | 21148 | 4: 94699823 | 21151 | 4: 94815446 | 21152 | 4: 94833611 |
| 21156 | 4: 94917698 | 21157 | 4: 94918493 | 21169 | 4: 95254763 | 21170 | 4: 95254765 | 21171 | 4: 95254773 |
| 21172 | 4: 95254787 | 21175 | 4: 95294696 | 21178 | 4: 95382163 | 21196 | 4: 95826773 | 21198 | 4: 95871966 |
| 21203 | 4: 95901052 | 21207 | 4: 95975966 | 21221 | 4: 96011612 | 21233 | 4: 96120257 | 21234 | 4: 96121708 |
| 21235 | 4: 96145894 | 21236 | 4: 96192829 | 21237 | 4: 96196621 | 21241 | 4: 96307908 | 21249 | 4: 96528346 |
| 21250 | 4: 96528347 | 21285 | 4: 96829783 | 21359 | 4: 96940681 | 21365 | 4: 96997475 | 21371 | 4: 97113666 |
| 21416 | 4: 97172490 | 21421 | 4: 97266645 | 21444 | 4: 97789995 | 21450 | 4: 98087559 | 21454 | 4: 98210913 |
| 21455 | 4: 98212198 | 21457 | 4: 98226178 | 21458 | 4: 98226699 | 21459 | 4: 98239198 | 21463 | 4: 98381525 |
| 21464 | 4: 98406747 | 21470 | 4: 98545217 | 21472 | 4: 98604946 | 21473 | 4: 98617473 | 21474 | 4: 98640953 |
| 21477 | 4: 98765309 | 21482 | 4: 98852463 | 21483 | 4: 98862279 | 21492 | 4: 99462535 | 21498 | 4: 99648977 |
| 21499 | 4: 99650381 | 21500 | 4: 99652215 | 21509 | 4: 99864130 | 21511 | 4: 99877799 | 21513 | 4: 99884272 |
| 21515 | 4: 99907629 | 21518 | 4: 100010231 | 21521 | 4: 100048120 | 21522 | 4: 100048151 | 21533 | 4: 100235968 |
| 21558 | 4: 100865583 | 21560 | 4: 100891716 | 21564 | 4: 100958472 | 21569 | 4: 101138513 | 21580 | 4: 101273627 |
| 21581 | 4: 101278348 | 21582 | 4: 101283299 | 21583 | 4: 101286906 | 21584 | 4: 101304065 | 21594 | 4: 101562975 |
| 21595 | 4: 101570088 | 21597 | 4: 101615078 | 21599 | 4: 101627868 | 21604 | 4: 101697277 | 21606 | 4: 101718193 |
| 21607 | 4: 101720088 | 21608 | 4: 101796334 | 21609 | 4: 101817565 | 21612 | 4: 102044239 | 21613 | 4: 102053938 |
| 21623 | 4: 102294876 | 21630 | 4: 102465082 | 21631 | 4: 102515585 | 21632 | 4: 102515711 | 21633 | 4: 102515830 |
| 21635 | 4: 102567199 | 21636 | 4: 102567675 | 21637 | 4: 102571979 | 21638 | 4: 102577247 | 21641 | 4: 102679178 |
| 21650 | 4: 102994861 | 21653 | 4: 103066617 | 21656 | 4: 103118208 | 21672 | 4: 103464890 | 21673 | 4: 103466759 |
| 21678 | 4: 103589488 | 21691 | 4: 103984758 | 21705 | 4: 104406614 | 21713 | 4: 104554874 | 21714 | 4: 104639191 |
| 21719 | 4: 104710790 | 21721 | 4: 104731929 | 21726 | 4: 104879688 | 21730 | 4: 104955413 | 21739 | 4: 105212815 |
| 21742 | 4: 105331039 | 21743 | 4: 105332159 | 21747 | 4: 105369148 | 21753 | 4: 105480650 | 21754 | 4: 105480728 |
| 21755 | 4: 105482792 | 21766 | 4: 105750600 | 21767 | 4: 105757195 | 21768 | 4: 105785525 | 21773 | 4: 106025379 |
| 21775 | 4: 106025551 | 21780 | 4: 106081660 | 21781 | 4: 106085160 | 21782 | 4: 106088179 | 21783 | 4: 106088193 |
| 21784 | 4: 106088224 | 21785 | 4: 106088251 | 21786 | 4: 106088286 | 21792 | 4: 106177289 | 21796 | 4: 106243037 |
| 21797 | 4: 106246162 | 21798 | 4: 106257365 | 21807 | 4: 106459877 | 21808 | 4: 106461237 | 21809 | 4: 106461523 |
| 21810 | 4: 106462584 | 21811 | 4: 106463211 | 21812 | 4: 106526851 | 21817 | 4: 106674077 | 21820 | 4: 106723818 |
| 21821 | 4: 106724580 | 21822 | 4: 106738338 | 21823 | 4: 106768265 | 21827 | 4: 106823063 | 21833 | 4: 106915810 |
| 21835 | 4: 106985422 | 21837 | 4: 106991677 | 21838 | 4: 106991678 | 21839 | 4: 106991818 | 21840 | 4: 106991819 |
| 21841 | 4: 106993054 | 21842 | 4: 106993083 | 21843 | 4: 106993086 | 21844 | 4: 106993163 | 21845 | 4: 106993484 |
| 21846 | 4: 106993491 | 21847 | 4: 106993778 | 21848 | 4: 106993951 | 21849 | 4: 106993975 | 21850 | 4: 106994109 |
| 21851 | 4: 106994258 | 21852 | 4: 106997706 | 21853 | 4: 106998207 | 21857 | 4: 107044582 | 21858 | 4: 107070723 |
| 21859 | 4: 107075139 | 21867 | 4: 107202696 | 21868 | 4: 107202723 | 21869 | 4: 107202867 | 21870 | 4: 107202876 |
| 21873 | 4: 107293705 | 21874 | 4: 107293767 | 21875 | 4: 107293937 | 21876 | 4: 107293975 | 21877 | 4: 107293984 |
| 21878 | 4: 107294409 | 21879 | 4: 107294413 | 21884 | 4: 107599054 | 21891 | 4: 107760331 | 21896 | 4: 107893188 |
| 21918 | 4: 108666311 | 21919 | 4: 108666329 | 21936 | 4: 109206944 | 21996 | 4: 110555410 | 21998 | 4: 110627140 |
| 22003 | 4: 110751708 | 22004 | 4: 110751716 | 22005 | 4: 110759035 | 22006 | 4: 110759043 | 22007 | 4: 110759085 |
| 22008 | 4: 110759138 | 22009 | 4: 110759193 | 22012 | 4: 110870537 | 22017 | 4: 110963804 | 22018 | 4: 110997685 |
| 22021 | 4: 111094939 | 22027 | 4: 111132995 | 22030 | 4: 111174187 | 22036 | 4: 111290634 | 22041 | 4: 111393405 |
| 22042 | 4: 111393524 | 22060 | 4: 111696373 | 22072 | 4: 111836637 | 22075 | 4: 111906579 | 22076 | 4: 111910434 |
| 22077 | 4: 111911436 | 22079 | 4: 111972681 | 22085 | 4: 112108098 | 22087 | 4: 112136295 | 22090 | 4: 112153870 |
| 22091 | 4: 112155488 | 22093 | 4: 112172784 | 22097 | 4: 112238690 | 22100 | 4: 112269261 | 22110 | 4: 112424206 |
| 22112 | 4: 112448562 | 22113 | 4: 112452844 | 22117 | 4: 112516104 | 22122 | 4: 112599976 | 22124 | 4: 112652763 |
| 22125 | 4: 112660638 | 22126 | 4: 112663309 | 22127 | 4: 112664440 | 22128 | 4: 112665144 | 22129 | 4: 112671210 |
| 22130 | 4: 112675707 | 22131 | 4: 112676199 | 22132 | 4: 112677340 | 22133 | 4: 112681999 | 22136 | 4: 112716249 |
| 22138 | 4: 112739929 | 22139 | 4: 112740655 | 22140 | 4: 112741810 | 22141 | 4: 112748029 | 22142 | 4: 112749294 |
| 22143 | 4: 112754237 | 22144 | 4: 112755579 | 22145 | 4: 112769671 | 22146 | 4: 112770319 | 22147 | 4: 112785850 |
| 22153 | 4: 112842045 | 22154 | 4: 112844573 | 22155 | 4: 112855696 | 22157 | 4: 112873667 | 22158 | 4: 112873671 |
| 22159 | 4: 112873791 | 22160 | 4: 112874161 | 22162 | 4: 112920884 | 22163 | 4: 112920908 | 22164 | 4: 112958565 |
| 22178 | 4: 113478902 | 22184 | 4: 113579221 | 22185 | 4: 113579506 | 22186 | 4: 113584318 | 22187 | 4: 113584327 |
| 22188 | 4: 113584399 | 22189 | 4: 113584405 | 22190 | 4: 113587147 | 22199 | 4: 113617600 | 22200 | 4: 113617762 |
| 22201 | 4: 113622976 | 22220 | 4: 113685923 | 22221 | 4: 113693137 | 22222 | 4: 113706536 | 22230 | 4: 113862937 |
| 22232 | 4: 113865696 | 22233 | 4: 113865709 | 22234 | 4: 113865711 | 22235 | 4: 113889872 | 22236 | 4: 113924572 |
| 22239 | 4: 113963854 | 22242 | 4: 114103350 | 22247 | 4: 114278681 | 22261 | 4: 114688072 | 22262 | 4: 114690368 |
| 22263 | 4: 114690442 | 22264 | 4: 114691485 | 22265 | 4: 114692365 | 22270 | 4: 114751599 | 22271 | 4: 114758803 |
| 22273 | 4: 114770151 | 22275 | 4: 114805260 | 22276 | 4: 114812473 | 22277 | 4: 114812501 | 22278 | 4: 114812550 |
| 22279 | 4: 114812583 | 22289 | 4: 115031057 | 22290 | 4: 115033034 | 22291 | 4: 115043261 | 22292 | 4: 115066503 |
| 22293 | 4: 115067836 | 22294 | 4: 115071402 | 22295 | 4: 115072381 | 22296 | 4: 115074258 | 22297 | 4: 115075256 |
| 22298 | 4: 115077110 | 22299 | 4: 115077972 | 22300 | 4: 115079711 | 22301 | 4: 115105025 | 22304 | 4: 115127050 |
| 22305 | 4: 115127065 | 22309 | 4: 115153886 | 22310 | 4: 115153896 | 22311 | 4: 115163025 | 22326 | 4: 115413253 |
| 22327 | 4: 115413261 | 22328 | 4: 115413321 | 22329 | 4: 115413386 | 22330 | 4: 115413395 | 22359 | 4: 116192647 |
| 22360 | 4: 116192669 | 22361 | 4: 116192689 | 22362 | 4: 116192738 | 22363 | 4: 116192764 | 22379 | 4: 116445855 |
| 22402 | 4: 116915804 | 22408 | 4: 117000567 | 22409 | 4: 117000602 | 22410 | 4: 117000603 | 22420 | 4: 117210404 |
| 22427 | 4: 117486610 | 22428 | 4: 117505997 | 22448 | 4: 117829938 | 22465 | 4: 118353876 | 22466 | 4: 118358762 |
| 22472 | 4: 118419883 | 22476 | 4: 118517071 | 22485 | 4: 118646844 | 22490 | 4: 118756391 | 22494 | 4: 118829611 |
| 22496 | 4: 118829788 | 22504 | 4: 118939017 | 22523 | 4: 119457121 | 22535 | 4: 119689809 | 22553 | 5: 77074 |
| 22579 | 5: 827590 | 22580 | 5: 834238 | 22581 | 5: 882265 | 22582 | 5: 882349 | 22583 | 5: 882384 |
| 22584 | 5: 903638 | 22597 | 5: 1099972 | 22608 | 5: 1345105 | 22610 | 5: 1356051 | 22611 | 5: 1356065 |
| 22612 | 5: 1356090 | 22613 | 5: 1356140 | 22614 | 5: 1356177 | 22615 | 5: 1356191 | 22618 | 5: 1456630 |
| 22623 | 5: 1589478 | 22625 | 5: 1618964 | 22670 | 5: 2514974 | 22681 | 5: 2735966 | 22684 | 5: 2800666 |
| 22698 | 5: 3349859 | 22699 | 5: 3349891 | 22719 | 5: 4068348 | 22749 | 5: 4663849 | 22757 | 5: 4889807 |
| 22768 | 5: 5162198 | 22770 | 5: 5188536 | 22772 | 5: 5196871 | 22778 | 5: 5266489 | 22780 | 5: 5300800 |
| 22789 | 5: 5528412 | 22792 | 5: 5566952 | 22804 | 5: 5799927 | 22805 | 5: 5817089 | 22814 | 5: 6016548 |
| 22822 | 5: 6285112 | 22826 | 5: 6336054 | 22828 | 5: 6436778 | 22838 | 5: 6632102 | 22859 | 5: 7063231 |
| 22871 | 5: 7268739 | 22873 | 5: 7277360 | 22874 | 5: 7277411 | 22877 | 5: 7377438 | 22895 | 5: 7885365 |
| 22915 | 5: 8557986 | 22928 | 5: 8826886 | 22932 | 5: 8934422 | 22933 | 5: 8934467 | 22934 | 5: 8934474 |
| 22962 | 5: 9693605 | 22964 | 5: 9730547 | 22967 | 5: 9797519 | 22968 | 5: 9803049 | 22969 | 5: 9856996 |
| 22970 | 5: 9857138 | 22971 | 5: 9858180 | 22976 | 5: 9982908 | 22978 | 5: 10011555 | 22980 | 5: 10029875 |
| 22981 | 5: 10034496 | 22989 | 5: 10156211 | 22991 | 5: 10201512 | 22992 | 5: 10252022 | 22993 | 5: 10278854 |
| 23007 | 5: 10506388 | 23014 | 5: 10670341 | 23020 | 5: 10828061 | 23021 | 5: 10843507 | 23022 | 5: 10843627 |
| 23023 | 5: 10844849 | 23031 | 5: 11089678 | 23063 | 5: 12028271 | 23067 | 5: 12102588 | 23068 | 5: 12103176 |
| 23073 | 5: 12156021 | 23074 | 5: 12156678 | 23083 | 5: 12325031 | 23084 | 5: 12326106 | 23096 | 5: 12460715 |
| 23097 | 5: 12473390 | 23105 | 5: 12528945 | 23111 | 5: 12719804 | 23121 | 5: 13036017 | 23124 | 5: 13069760 |
| 23139 | 5: 13541155 | 23151 | 5: 14006938 | 23155 | 5: 14124180 | 23171 | 5: 14599931 | 23172 | 5: 14600013 |
| 23175 | 5: 14680469 | 23195 | 5: 15100766 | 23199 | 5: 15173290 | 23216 | 5: 15462968 | 23219 | 5: 15566954 |
| 23220 | 5: 15566955 | 23221 | 5: 15566965 | 23222 | 5: 15566966 | 23223 | 5: 15566972 | 23224 | 5: 15566977 |
| 23225 | 5: 15567052 | 23226 | 5: 15567070 | 23227 | 5: 15567096 | 23228 | 5: 15567111 | 23232 | 5: 15704626 |
| 23266 | 5: 16709205 | 23270 | 5: 16800463 | 23282 | 5: 17119922 | 23285 | 5: 17174787 | 23288 | 5: 17241349 |
| 23293 | 5: 17398558 | 23306 | 5: 17895598 | 23307 | 5: 17903579 | 23318 | 5: 18353350 | 23321 | 5: 18410507 |
| 23322 | 5: 18410599 | 23338 | 5: 18941613 | 23351 | 5: 19280880 | 23359 | 5: 19568730 | 23374 | 5: 19885896 |
| 23376 | 5: 19976107 | 23377 | 5: 19993960 | 23378 | 5: 20014240 | 23381 | 5: 20097603 | 23382 | 5: 20149313 |
| 23384 | 5: 20195300 | 23389 | 5: 20436830 | 23396 | 5: 20590589 | 23399 | 5: 20732321 | 23405 | 5: 20832737 |
| 23406 | 5: 20832885 | 23407 | 5: 20833047 | 23409 | 5: 20843567 | 23410 | 5: 20910258 | 23411 | 5: 20936220 |
| 23416 | 5: 21019650 | 23422 | 5: 21196220 | 23445 | 5: 21745511 | 23447 | 5: 21797550 | 23452 | 5: 21874789 |
| 23454 | 5: 21920920 | 23466 | 5: 22115062 | 23473 | 5: 22298578 | 23474 | 5: 22323880 | 23477 | 5: 22412419 |
| 23487 | 5: 22513497 | 23491 | 5: 22558133 | 23492 | 5: 22558154 | 23493 | 5: 22558155 | 23502 | 5: 22837489 |
| 23503 | 5: 22837951 | 23507 | 5: 22898017 | 23509 | 5: 22944167 | 23510 | 5: 22947768 | 23514 | 5: 23017065 |
| 23515 | 5: 23045588 | 23517 | 5: 23055987 | 23524 | 5: 23260695 | 23525 | 5: 23261205 | 23526 | 5: 23269298 |
| 23530 | 5: 23386932 | 23533 | 5: 23440852 | 23534 | 5: 23449942 | 23542 | 5: 23608812 | 23545 | 5: 23736650 |
| 23548 | 5: 23753028 | 23552 | 5: 23816911 | 23556 | 5: 23949886 | 23561 | 5: 23975472 | 23570 | 5: 24168834 |
| 23578 | 5: 24352659 | 23579 | 5: 24352754 | 23587 | 5: 24564540 | 23591 | 5: 24706081 | 23594 | 5: 24861511 |
| 23595 | 5: 24861917 | 23596 | 5: 24862040 | 23597 | 5: 24863117 | 23598 | 5: 24864988 | 23599 | 5: 24865092 |
| 23610 | 5: 25042104 | 23618 | 5: 25424142 | 23619 | 5: 25424207 | 23622 | 5: 25531432 | 23627 | 5: 25660326 |
| 23629 | 5: 25668094 | 23632 | 5: 25770487 | 23635 | 5: 25865044 | 23641 | 5: 25948717 | 23642 | 5: 25953449 |
| 23648 | 5: 26274617 | 23652 | 5: 26346656 | 23653 | 5: 26359865 | 23654 | 5: 26372843 | 23655 | 5: 26380803 |
| 23661 | 5: 26513500 | 23662 | 5: 26516803 | 23663 | 5: 26527639 | 23664 | 5: 26531218 | 23672 | 5: 26728680 |
| 23674 | 5: 26750096 | 23679 | 5: 26881116 | 23684 | 5: 27043324 | 23685 | 5: 27062257 | 23686 | 5: 27098658 |
| 23700 | 5: 27397526 | 23705 | 5: 27478824 | 23712 | 5: 27611131 | 23718 | 5: 27732993 | 23724 | 5: 27782046 |
| 23726 | 5: 27821648 | 23730 | 5: 27894859 | 23736 | 5: 28067367 | 23738 | 5: 28076547 | 23741 | 5: 28125774 |
| 23750 | 5: 28398456 | 23754 | 5: 28468312 | 23755 | 5: 28474788 | 23770 | 5: 28759211 | 23771 | 5: 28764621 |
| 23776 | 5: 28916451 | 23779 | 5: 28965670 | 23787 | 5: 29216390 | 23792 | 5: 29377131 | 23809 | 5: 29727121 |
| 23815 | 5: 29862958 | 23819 | 5: 29973188 | 23830 | 5: 30143566 | 23838 | 5: 30392463 | 23844 | 5: 30521361 |
| 23845 | 5: 30526943 | 23846 | 5: 30564929 | 23847 | 5: 30571021 | 23848 | 5: 30583097 | 23849 | 5: 30584287 |
| 23852 | 5: 30656853 | 23861 | 5: 30885641 | 23863 | 5: 30907588 | 23864 | 5: 30945722 | 23869 | 5: 31116777 |
| 23890 | 5: 31693587 | 23899 | 5: 32136613 | 23912 | 5: 32401173 | 23913 | 5: 32414358 | 23916 | 5: 32527988 |
| 23917 | 5: 32544707 | 23918 | 5: 32549914 | 23921 | 5: 32589543 | 23931 | 5: 32826960 | 23939 | 5: 33070073 |
| 23941 | 5: 33080199 | 23951 | 5: 33311326 | 23955 | 5: 33354800 | 23962 | 5: 33573362 | 23971 | 5: 33769971 |
| 23972 | 5: 33803288 | 23979 | 5: 33964757 | 23980 | 5: 33974899 | 24008 | 5: 34894503 | 24010 | 5: 34992883 |
| 24011 | 5: 34992887 | 24012 | 5: 34992888 | 24014 | 5: 35004904 | 24015 | 5: 35004932 | 24016 | 5: 35039187 |
| 24017 | 5: 35055317 | 24018 | 5: 35080229 | 24022 | 5: 35207804 | 24033 | 5: 35577195 | 24036 | 5: 35771527 |
| 24066 | 5: 36566052 | 24079 | 5: 37113142 | 24101 | 5: 37695072 | 24102 | 5: 37711059 | 24104 | 5: 37735001 |
| 24109 | 5: 37845630 | 24113 | 5: 37881472 | 24119 | 5: 37991676 | 24122 | 5: 38029636 | 24123 | 5: 38033300 |
| 24125 | 5: 38058027 | 24127 | 5: 38106278 | 24129 | 5: 38114225 | 24130 | 5: 38116179 | 24131 | 5: 38135398 |
| 24134 | 5: 38174231 | 24144 | 5: 38453061 | 24145 | 5: 38483708 | 24146 | 5: 38527379 | 24147 | 5: 38531595 |
| 24148 | 5: 38544831 | 24150 | 5: 38593359 | 24163 | 5: 39154317 | 24165 | 5: 39228037 | 24166 | 5: 39257315 |
| 24186 | 5: 39750764 | 24202 | 5: 40183299 | 24204 | 5: 40237324 | 24207 | 5: 40321836 | 24208 | 5: 40321842 |
| 24209 | 5: 40321905 | 24210 | 5: 40321940 | 24211 | 5: 40322458 | 24212 | 5: 40322491 | 24214 | 5: 40326831 |
| 24215 | 5: 40340341 | 24216 | 5: 40342525 | 24227 | 5: 40812862 | 24251 | 5: 41504706 | 24264 | 5: 41958537 |
| 24265 | 5: 41958597 | 24266 | 5: 41958622 | 24267 | 5: 41987705 | 24270 | 5: 42145772 | 24282 | 5: 42611303 |
| 24291 | 5: 42843742 | 24296 | 5: 42890909 | 24302 | 5: 43044298 | 24305 | 5: 43082557 | 24314 | 5: 43415290 |
| 24315 | 5: 43480306 | 24318 | 5: 43506882 | 24324 | 5: 43599378 | 24331 | 5: 43811761 | 24334 | 5: 43863604 |
| 24335 | 5: 43864090 | 24336 | 5: 43864115 | 24337 | 5: 43869544 | 24338 | 5: 43875060 | 24339 | 5: 43878846 |
| 24340 | 5: 43880997 | 24341 | 5: 43881099 | 24342 | 5: 43883078 | 24343 | 5: 43897037 | 24344 | 5: 43897095 |
| 24346 | 5: 43901763 | 24350 | 5: 44040614 | 24351 | 5: 44049829 | 24360 | 5: 44388064 | 24374 | 5: 44804564 |
| 24377 | 5: 44906731 | 24384 | 5: 45175795 | 24391 | 5: 45366260 | 24400 | 5: 45549099 | 24403 | 5: 45619287 |
| 24414 | 5: 45917363 | 24422 | 5: 46063715 | 24424 | 5: 46103767 | 24425 | 5: 46103871 | 24435 | 5: 46335844 |
| 24440 | 5: 46443847 | 24442 | 5: 46516496 | 24483 | 5: 47241615 | 24489 | 5: 47478077 | 24492 | 5: 47556051 |
| 24494 | 5: 47596785 | 24507 | 5: 48011805 | 24509 | 5: 48115271 | 24513 | 5: 48219521 | 24521 | 5: 48357494 |
| 24546 | 5: 49071305 | 24547 | 5: 49121969 | 24548 | 5: 49128930 | 24551 | 5: 49244193 | 24555 | 5: 49443860 |
| 24557 | 5: 49480432 | 24576 | 5: 50003215 | 24577 | 5: 50004728 | 24587 | 5: 50241038 | 24593 | 5: 50468141 |
| 24601 | 5: 50729869 | 24604 | 5: 50805474 | 24606 | 5: 50826119 | 24612 | 5: 50987421 | 24613 | 5: 50995228 |
| 24615 | 5: 51012775 | 24616 | 5: 51014179 | 24617 | 5: 51026281 | 24618 | 5: 51028777 | 24619 | 5: 51028823 |
| 24620 | 5: 51045771 | 24621 | 5: 51046494 | 24622 | 5: 51046653 | 24623 | 5: 51050849 | 24624 | 5: 51055590 |
| 24625 | 5: 51056409 | 24626 | 5: 51074757 | 24627 | 5: 51076137 | 24629 | 5: 51106212 | 24630 | 5: 51125457 |
| 24633 | 5: 51222282 | 24639 | 5: 51428229 | 24649 | 5: 51662795 | 24651 | 5: 51737595 | 24652 | 5: 51807509 |
| 24660 | 5: 52059878 | 24661 | 5: 52059908 | 24662 | 5: 52059930 | 24680 | 5: 52526329 | 24693 | 5: 53179786 |
| 24711 | 5: 53766718 | 24712 | 5: 53779538 | 24713 | 5: 53781567 | 24714 | 5: 53786730 | 24717 | 5: 53862521 |
| 24723 | 5: 54013034 | 24732 | 5: 54299758 | 24733 | 5: 54301166 | 24734 | 5: 54304681 | 24735 | 5: 54306088 |
| 24736 | 5: 54309409 | 24737 | 5: 54324570 | 24738 | 5: 54332158 | 24740 | 5: 54358159 | 24741 | 5: 54367083 |
| 24742 | 5: 54370187 | 24743 | 5: 54376482 | 24745 | 5: 54399560 | 24748 | 5: 54511599 | 24749 | 5: 54514698 |
| 24751 | 5: 54563791 | 24755 | 5: 54632888 | 24761 | 5: 54765009 | 24785 | 5: 55509920 | 24789 | 5: 55583411 |
| 24790 | 5: 55619423 | 24791 | 5: 55634685 | 24792 | 5: 55640765 | 24793 | 5: 55652450 | 24795 | 5: 55670462 |
| 24797 | 5: 55696567 | 24798 | 5: 55702652 | 24799 | 5: 55707061 | 24802 | 5: 55873933 | 24803 | 5: 55879578 |
| 24805 | 5: 55940985 | 24807 | 5: 55956661 | 24848 | 5: 56720568 | 24849 | 5: 56792015 | 24853 | 5: 56910201 |
| 24854 | 5: 56953231 | 24859 | 5: 57100134 | 24864 | 5: 57204918 | 24873 | 5: 57427084 | 24882 | 5: 57667264 |
| 24883 | 5: 57672067 | 24896 | 5: 58068683 | 24904 | 5: 58250121 | 24905 | 5: 58285910 | 24915 | 5: 58749685 |
| 24923 | 5: 59304558 | 24925 | 5: 59313596 | 24929 | 5: 59364960 | 24930 | 5: 59365133 | 24941 | 5: 59865035 |
| 24950 | 5: 60107106 | 24956 | 5: 60233686 | 24958 | 5: 60262488 | 24960 | 5: 60297442 | 24962 | 5: 60326833 |
| 24965 | 5: 60380312 | 24971 | 5: 60510721 | 24972 | 5: 60532648 | 24973 | 5: 60543258 | 24975 | 5: 60557011 |
| 24977 | 5: 60595067 | 24983 | 5: 60718806 | 24985 | 5: 60758142 | 24986 | 5: 60766663 | 24987 | 5: 60766858 |
| 24996 | 5: 61052215 | 25025 | 5: 62076373 | 25033 | 5: 62426367 | 25040 | 5: 62518142 | 25043 | 5: 62636512 |
| 25051 | 5: 62933839 | 25052 | 5: 62941170 | 25083 | 5: 63542114 | 25095 | 5: 63850062 | 25104 | 5: 64226221 |
| 25106 | 5: 64234434 | 25118 | 5: 64619628 | 25137 | 5: 65171005 | 25138 | 5: 65183093 | 25142 | 5: 65232220 |
| 25151 | 5: 65446380 | 25154 | 5: 65535107 | 25155 | 5: 65542737 | 25156 | 5: 65543322 | 25157 | 5: 65543372 |
| 25167 | 5: 65703423 | 25168 | 5: 65712111 | 25169 | 5: 65751283 | 25170 | 5: 65752854 | 25172 | 5: 65760426 |
| 25173 | 5: 65761371 | 25179 | 5: 66009583 | 25195 | 5: 66518756 | 25225 | 5: 67447645 | 25226 | 5: 67449152 |
| 25227 | 5: 67463576 | 25228 | 5: 67465088 | 25229 | 5: 67468026 | 25230 | 5: 67470940 | 25231 | 5: 67473722 |
| 25232 | 5: 67475068 | 25233 | 5: 67475725 | 25234 | 5: 67477065 | 25235 | 5: 67481131 | 25237 | 5: 67514487 |
| 25238 | 5: 67526075 | 25239 | 5: 67526864 | 25241 | 5: 67555054 | 25242 | 5: 67563065 | 25243 | 5: 67568345 |
| 25251 | 5: 67846005 | 25252 | 5: 67848289 | 25253 | 5: 67851400 | 25260 | 5: 68044942 | 25261 | 5: 68062320 |
| 25265 | 5: 68180283 | 25267 | 5: 68195298 | 25277 | 5: 68311976 | 25280 | 5: 68341993 | 25282 | 5: 68419217 |
| 25284 | 5: 68527931 | 25292 | 5: 68745462 | 25294 | 5: 68847086 | 25305 | 5: 68998098 | 25309 | 5: 69114492 |
| 25314 | 5: 69191792 | 25315 | 5: 69211669 | 25318 | 5: 69282729 | 25319 | 5: 69295982 | 25327 | 5: 69507467 |
| 25329 | 5: 69534170 | 25330 | 5: 69540325 | 25339 | 5: 69690113 | 25346 | 5: 69807021 | 25354 | 5: 70143301 |
| 25369 | 5: 70657641 | 25371 | 5: 70725053 | 25383 | 5: 70904784 | 25385 | 5: 70931624 | 25388 | 5: 71041333 |
| 25390 | 5: 71113115 | 25393 | 5: 71147255 | 25397 | 5: 71158676 | 25399 | 5: 71204940 | 25401 | 5: 71215860 |
| 25402 | 5: 71247169 | 25404 | 5: 71274477 | 25406 | 5: 71300632 | 25408 | 5: 71346087 | 25409 | 5: 71377241 |
| 25418 | 5: 71535392 | 25422 | 5: 71629712 | 25432 | 5: 71856285 | 25433 | 5: 71893342 | 25434 | 5: 71896515 |
| 25435 | 5: 71909707 | 25438 | 5: 71958333 | 25442 | 5: 72039709 | 25443 | 5: 72058094 | 25445 | 5: 72064330 |
| 25458 | 5: 72293857 | 25461 | 5: 72366005 | 25462 | 5: 72368884 | 25475 | 5: 72696496 | 25500 | 5: 73554593 |
| 25504 | 5: 73672740 | 25521 | 5: 73884415 | 25522 | 5: 73908825 | 25523 | 5: 73980075 | 25529 | 5: 74150522 |
| 25532 | 5: 74225942 | 25533 | 5: 74245072 | 25545 | 5: 74650187 | 25547 | 5: 74725927 | 25549 | 5: 74742101 |
| 25555 | 5: 74908285 | 25556 | 5: 74908338 | 25557 | 5: 74909826 | 25558 | 5: 74911263 | 25559 | 5: 74911341 |
| 25560 | 5: 74915786 | 25561 | 5: 74919175 | 25562 | 5: 74925227 | 25563 | 5: 74925818 | 25564 | 5: 74926028 |
| 25565 | 5: 74928887 | 25566 | 5: 74936574 | 25571 | 5: 75167453 | 25574 | 5: 75224410 | 25575 | 5: 75224450 |
| 25576 | 5: 75224458 | 25577 | 5: 75224463 | 25579 | 5: 75224524 | 25582 | 5: 75261093 | 25586 | 5: 75281166 |
| 25599 | 5: 75676623 | 25605 | 5: 75823353 | 25608 | 5: 75912892 | 25609 | 5: 75919398 | 25614 | 5: 75988734 |
| 25620 | 5: 76209127 | 25621 | 5: 76269564 | 25622 | 5: 76269603 | 25631 | 5: 76640050 | 25632 | 5: 76666195 |
| 25640 | 5: 76784217 | 25649 | 5: 77156529 | 25650 | 5: 77161120 | 25651 | 5: 77180731 | 25652 | 5: 77193464 |
| 25653 | 5: 77193475 | 25654 | 5: 77193500 | 25660 | 5: 77277820 | 25661 | 5: 77302753 | 25662 | 5: 77331990 |
| 25664 | 5: 77340074 | 25666 | 5: 77362757 | 25667 | 5: 77410429 | 25677 | 5: 77928157 | 25678 | 5: 77928784 |
| 25692 | 5: 78324681 | 25698 | 5: 78506395 | 25699 | 5: 78510349 | 25700 | 5: 78522919 | 25708 | 5: 78820352 |
| 25709 | 5: 78820587 | 25710 | 5: 78820588 | 25711 | 5: 78820618 | 25712 | 5: 78820726 | 25713 | 5: 78820795 |
| 25714 | 5: 78820814 | 25715 | 5: 78821054 | 25718 | 5: 78829751 | 25719 | 5: 78832107 | 25720 | 5: 78832141 |
| 25721 | 5: 78832271 | 25752 | 5: 79910151 | 25775 | 5: 80693563 | 25784 | 5: 80982109 | 25788 | 5: 81166934 |
| 25794 | 5: 81369156 | 25802 | 5: 81505086 | 25816 | 5: 82023846 | 25818 | 5: 82073695 | 25822 | 5: 82125171 |
| 25825 | 5: 82161343 | 25835 | 5: 82321735 | 25839 | 5: 82419134 | 25842 | 5: 82485421 | 25843 | 5: 82485429 |
| 25844 | 5: 82491588 | 25847 | 5: 82535021 | 25868 | 5: 82755673 | 25874 | 5: 83018943 | 25879 | 5: 83102062 |
| 25885 | 5: 83210516 | 25887 | 5: 83258916 | 25890 | 5: 83347445 | 25919 | 5: 83920522 | 25924 | 5: 84024864 |
| 26062 | 5: 84271561 | 26081 | 5: 84723773 | 26087 | 5: 84917552 | 26096 | 5: 85106613 | 26097 | 5: 85112695 |
| 26102 | 5: 85286730 | 26138 | 5: 86172964 | 26151 | 5: 86508471 | 26155 | 5: 86538135 | 26167 | 5: 86803983 |
| 26168 | 5: 86807041 | 26195 | 5: 87419158 | 26197 | 5: 87436653 | 26205 | 5: 87558801 | 26222 | 5: 87935293 |
| 26227 | 5: 88025590 | 26232 | 5: 88169363 | 26240 | 5: 88321354 | 26241 | 5: 88337164 | 26246 | 5: 88393821 |
| 26251 | 5: 88442816 | 26257 | 5: 88527567 | 26259 | 5: 88560515 | 26260 | 5: 88562589 | 26264 | 5: 88656117 |
| 26265 | 5: 88656655 | 26266 | 5: 88663005 | 26267 | 5: 88664137 | 26268 | 5: 88664970 | 26269 | 5: 88665685 |
| 26270 | 5: 88667764 | 26271 | 5: 88668736 | 26272 | 5: 88669208 | 26273 | 5: 88670166 | 26274 | 5: 88670949 |
| 26275 | 5: 88671581 | 26276 | 5: 88672838 | 26277 | 5: 88673518 | 26278 | 5: 88675186 | 26279 | 5: 88676434 |
| 26280 | 5: 88677664 | 26281 | 5: 88679714 | 26282 | 5: 88681101 | 26289 | 5: 88791076 | 26294 | 5: 88871429 |
| 26295 | 5: 88903163 | 26297 | 5: 88927675 | 26303 | 5: 89099225 | 26311 | 5: 89264046 | 26313 | 5: 89283625 |
| 26315 | 5: 89343653 | 26316 | 5: 89359278 | 26317 | 5: 89364312 | 26327 | 5: 89668965 | 26337 | 5: 89886383 |
| 26338 | 5: 89886385 | 26346 | 5: 90068114 | 26353 | 5: 90321950 | 26354 | 5: 90345643 | 26356 | 5: 90409538 |
| 26363 | 5: 90604091 | 26369 | 5: 90678336 | 26375 | 5: 90778808 | 26425 | 5: 92149419 | 26439 | 5: 92379543 |
| 26479 | 5: 92796508 | 26480 | 5: 92796509 | 26481 | 5: 92796641 | 26482 | 5: 92796716 | 26486 | 5: 92864262 |
| 26490 | 5: 92953627 | 26493 | 5: 93100450 | 26512 | 5: 93520661 | 26515 | 5: 93603557 | 26517 | 5: 93614794 |
| 26539 | 5: 94115151 | 26543 | 5: 94190519 | 26550 | 5: 94345725 | 26551 | 5: 94346916 | 26555 | 5: 94480126 |
| 26560 | 5: 94697047 | 26567 | 5: 94796315 | 26568 | 5: 94806990 | 26586 | 5: 95221444 | 26588 | 5: 95236526 |
| 26590 | 5: 95266607 | 26592 | 5: 95275712 | 26609 | 5: 95732385 | 26613 | 5: 95783150 | 26623 | 5: 96041063 |
| 26630 | 5: 96115730 | 26634 | 5: 96229534 | 26636 | 5: 96266124 | 26641 | 5: 96344421 | 26648 | 5: 96479304 |
| 26650 | 5: 96555471 | 26653 | 5: 96570693 | 26654 | 5: 96587114 | 26657 | 5: 96683112 | 26658 | 5: 96697638 |
| 26659 | 5: 96722002 | 26663 | 5: 96826963 | 26665 | 5: 96891834 | 26666 | 5: 96895092 | 26667 | 5: 96897342 |
| 26668 | 5: 96903857 | 26669 | 5: 96904695 | 26670 | 5: 96905308 | 26672 | 5: 96908444 | 26673 | 5: 96909315 |
| 26678 | 5: 96986050 | 26682 | 5: 97026543 | 26686 | 5: 97080334 | 26696 | 5: 97322489 | 26701 | 5: 97423919 |
| 26702 | 5: 97434109 | 26704 | 5: 97466504 | 26705 | 5: 97487627 | 26710 | 5: 97624543 | 26727 | 5: 98048878 |
| 26731 | 5: 98072135 | 26741 | 5: 98284200 | 26743 | 5: 98321751 | 26746 | 5: 98348925 | 26747 | 5: 98360313 |
| 26748 | 5: 98369761 | 26753 | 5: 98558774 | 26754 | 5: 98558789 | 26755 | 5: 98558837 | 26756 | 5: 98558879 |
| 26757 | 5: 98559154 | 26758 | 5: 98559190 | 26759 | 5: 98559458 | 26760 | 5: 98559459 | 26762 | 5: 98571338 |
| 26769 | 5: 98750219 | 26771 | 5: 98819283 | 26774 | 5: 98902747 | 26779 | 5: 98952191 | 26782 | 5: 98988266 |
| 26783 | 5: 98991352 | 26786 | 5: 99023106 | 26793 | 5: 99430794 | 26800 | 5: 99597967 | 26801 | 5: 99613789 |
| 26803 | 5: 99657462 | 26804 | 5: 99657533 | 26805 | 5: 99657651 | 26806 | 5: 99657668 | 26808 | 5: 99674708 |
| 26809 | 5: 99678222 | 26811 | 5: 99698551 | 26812 | 5: 99698552 | 26815 | 5: 99764163 | 26817 | 5: 99773482 |
| 26819 | 5: 99784430 | 26827 | 5: 99916927 | 26837 | 5: 100075903 | 26838 | 5: 100085604 | 26839 | 5: 100089578 |
| 26841 | 5: 100109898 | 26842 | 5: 100126206 | 26843 | 5: 100133218 | 26846 | 5: 100234492 | 26848 | 5: 100300261 |
| 26849 | 5: 100311514 | 26850 | 5: 100341741 | 26852 | 5: 100377385 | 26854 | 5: 100381117 | 26856 | 5: 100389557 |
| 26858 | 5: 100426926 | 26866 | 5: 100696501 | 26867 | 5: 100696607 | 26868 | 5: 100698121 | 26869 | 5: 100698579 |
| 26872 | 5: 100728733 | 26881 | 5: 100884953 | 26901 | 5: 101256059 | 26903 | 5: 101355705 | 26904 | 5: 101371243 |
| 26906 | 5: 101513339 | 26911 | 5: 101595246 | 26915 | 5: 101614862 | 26916 | 5: 101616220 | 26927 | 5: 101872342 |
| 26929 | 5: 101932704 | 26930 | 5: 101932771 | 26942 | 5: 102469703 | 26956 | 5: 103101288 | 26957 | 5: 103104040 |
| 26959 | 5: 103119311 | 26960 | 5: 103119357 | 26961 | 5: 103119383 | 26962 | 5: 103119408 | 26963 | 5: 103119413 |
| 26965 | 5: 103127016 | 26969 | 5: 103180670 | 26970 | 5: 103181148 | 26971 | 5: 103186080 | 26972 | 5: 103186163 |
| 26975 | 5: 103229995 | 26976 | 5: 103292486 | 26977 | 5: 103293544 | 26979 | 5: 103324283 | 26987 | 5: 103577479 |
| 26992 | 5: 103749650 | 26993 | 5: 103753540 | 26994 | 5: 103765906 | 27009 | 5: 104024017 | 27010 | 5: 104047698 |
| 27011 | 5: 104049926 | 27012 | 5: 104051208 | 27014 | 5: 104058009 | 27015 | 5: 104067878 | 27016 | 5: 104090665 |
| 27019 | 5: 104166543 | 27022 | 5: 104306581 | 27027 | 5: 104414866 | 27028 | 5: 104421461 | 27029 | 5: 104425156 |
| 27030 | 5: 104425652 | 27035 | 5: 104475838 | 27038 | 5: 104487779 | 27048 | 5: 104725697 | 27052 | 5: 104812593 |
| 27057 | 5: 104944974 | 27059 | 5: 104962040 | 27065 | 5: 105070263 | 27066 | 5: 105095351 | 27070 | 5: 105138348 |
| 27071 | 5: 105144347 | 27088 | 5: 105596087 | 27097 | 5: 105756724 | 27103 | 5: 105773809 | 27107 | 5: 105778777 |
| 27108 | 5: 105779368 | 27151 | 5: 106320490 | 27154 | 5: 106351560 | 27160 | 5: 106474477 | 27171 | 5: 106583604 |
| 27173 | 5: 106588115 | 27177 | 5: 106701615 | 27184 | 5: 106816406 | 27190 | 5: 106904200 | 27191 | 5: 106904337 |
| 27192 | 5: 106904427 | 27193 | 5: 106904456 | 27194 | 5: 106906152 | 27196 | 5: 106921443 | 27198 | 5: 106952814 |
| 27199 | 5: 106952815 | 27208 | 5: 107090339 | 27210 | 5: 107152886 | 27211 | 5: 107155008 | 27213 | 5: 107195685 |
| 27214 | 5: 107203743 | 27226 | 5: 107421295 | 27230 | 5: 107445396 | 27236 | 5: 107578561 | 27239 | 5: 107683951 |
| 27242 | 5: 107725814 | 27258 | 5: 108050852 | 27260 | 5: 108055831 | 27261 | 5: 108055990 | 27264 | 5: 108099551 |
| 27266 | 5: 108207411 | 27267 | 5: 108207896 | 27270 | 5: 108230364 | 27272 | 5: 108235905 | 27277 | 5: 108323867 |
| 27296 | 5: 108547597 | 27312 | 5: 108942683 | 27314 | 5: 108955049 | 27316 | 5: 108984906 | 27320 | 5: 109034391 |
| 27322 | 5: 109075632 | 27323 | 5: 109090548 | 27324 | 5: 109096526 | 27325 | 5: 109153205 | 27327 | 5: 109154168 |
| 27328 | 5: 109178144 | 27329 | 5: 109178358 | 27330 | 5: 109179308 | 27335 | 5: 109320605 | 27339 | 5: 109412747 |
| 27348 | 5: 109611685 | 27349 | 5: 109615284 | 27352 | 5: 109687895 | 27354 | 5: 109722487 | 27356 | 5: 109749503 |
| 27357 | 5: 109754096 | 27361 | 5: 109808607 | 27362 | 5: 109847158 | 27368 | 5: 109988776 | 27369 | 5: 109989550 |
| 27371 | 5: 110069248 | 27375 | 5: 110112327 | 27379 | 5: 110193224 | 27383 | 5: 110314027 | 27384 | 5: 110353226 |
| 27385 | 5: 110369778 | 27386 | 5: 110373456 | 27387 | 5: 110373555 | 27389 | 5: 110374859 | 27393 | 5: 110398433 |
| 27397 | 5: 110509525 | 27400 | 5: 110531320 | 27401 | 5: 110531367 | 27402 | 5: 110532648 | 27403 | 5: 110532703 |
| 27404 | 5: 110532709 | 27405 | 5: 110533852 | 27406 | 5: 110533859 | 27407 | 5: 110534455 | 27410 | 5: 110564870 |
| 27412 | 5: 110569021 | 27413 | 5: 110569141 | 27414 | 5: 110569146 | 27419 | 5: 110751512 | 27431 | 5: 111053132 |
| 27433 | 5: 111063936 | 27434 | 5: 111065855 | 27435 | 5: 111065875 | 27446 | 5: 111321416 | 27452 | 5: 111501396 |
| 27453 | 5: 111502809 | 27460 | 5: 111753901 | 27465 | 5: 111845063 | 27468 | 5: 111862953 | 27469 | 5: 111864267 |
| 27480 | 5: 112115889 | 27481 | 5: 112115946 | 27482 | 5: 112126293 | 27483 | 5: 112126298 | 27484 | 5: 112133653 |
| 27485 | 5: 112135929 | 27489 | 5: 112235754 | 27492 | 5: 112343380 | 27495 | 5: 112408202 | 27498 | 5: 112448429 |
| 27499 | 5: 112455682 | 27505 | 5: 112604562 | 27507 | 5: 112634025 | 27508 | 5: 112641994 | 27510 | 5: 112722034 |
| 27513 | 5: 112805426 | 27516 | 5: 112917350 | 27527 | 5: 113148146 | 27528 | 5: 113148464 | 27529 | 5: 113149795 |
| 27550 | 5: 113769146 | 27556 | 5: 113920144 | 27558 | 5: 113928017 | 27564 | 5: 114059697 | 27573 | 5: 114343422 |
| 27574 | 5: 114343454 | 27577 | 5: 114569421 | 27581 | 5: 114723102 | 27590 | 5: 115002143 | 27593 | 5: 115052949 |
| 27600 | 5: 115255504 | 27601 | 5: 115265500 | 27608 | 5: 115466163 | 27615 | 5: 115514965 | 27616 | 5: 115517449 |
| 27626 | 5: 115682503 | 27629 | 5: 115766557 | 27651 | 5: 116121476 | 27661 | 5: 116390999 | 27663 | 5: 116423647 |
| 27665 | 5: 116464508 | 27669 | 5: 116511553 | 27672 | 5: 116537986 | 27674 | 5: 116577132 | 27685 | 5: 116593122 |
| 27686 | 5: 116593191 | 27687 | 5: 116593192 | 27689 | 5: 116593706 | 27690 | 5: 116593715 | 27691 | 5: 116593762 |
| 27692 | 5: 116593794 | 27693 | 5: 116593850 | 27703 | 5: 116596428 | 27704 | 5: 116596441 | 27705 | 5: 116596625 |
| 27709 | 5: 116597945 | 27710 | 5: 116598050 | 27713 | 5: 116600211 | 27714 | 5: 116601776 | 27717 | 5: 116622560 |
| 27723 | 5: 116699001 | 27724 | 5: 116699079 | 27725 | 5: 116699138 | 27726 | 5: 116699145 | 27727 | 5: 116699159 |
| 27732 | 5: 116743643 | 27734 | 5: 116745570 | 27735 | 5: 116757705 | 27740 | 5: 116908657 | 27741 | 5: 116911216 |
| 27742 | 5: 116911371 | 27743 | 5: 116911376 | 27744 | 5: 116915304 | 27745 | 5: 116915313 | 27746 | 5: 116915402 |
| 27748 | 5: 116922121 | 27751 | 5: 116973879 | 27753 | 5: 116989243 | 27754 | 5: 116991259 | 27762 | 5: 117097690 |
| 27767 | 5: 117234102 | 27769 | 5: 117262549 | 27770 | 5: 117262555 | 27771 | 5: 117262556 | 27773 | 5: 117262630 |
| 27785 | 5: 117459589 | 27786 | 5: 117469696 | 27787 | 5: 117471979 | 27788 | 5: 117473591 | 27789 | 5: 117474224 |
| 27790 | 5: 117475215 | 27791 | 5: 117476538 | 27799 | 5: 117579793 | 27805 | 5: 117645005 | 27815 | 5: 117785747 |
| 27818 | 5: 117850396 | 27844 | 5: 118401624 | 27854 | 5: 118620070 | 27859 | 5: 118696815 | 27875 | 5: 118913308 |
| 27879 | 5: 118954058 | 27880 | 5: 118959454 | 27881 | 5: 118971379 | 27882 | 5: 118972496 | 27883 | 5: 118978705 |
| 27885 | 5: 118982714 | 27900 | 5: 119181604 | 27915 | 5: 119570841 | 27918 | 5: 119667915 | 27921 | 5: 119771063 |
| 27922 | 5: 119774093 | 27923 | 5: 119775410 | 27926 | 5: 119853084 | 27931 | 5: 119945721 | 27932 | 5: 119995446 |
| 27964 | 6: 844150 | 27965 | 6: 844153 | 27972 | 6: 1031446 | 27980 | 6: 1285962 | 27990 | 6: 1702526 |
| 27992 | 6: 1761466 | 27993 | 6: 1761542 | 27994 | 6: 1761563 | 27995 | 6: 1774727 | 27998 | 6: 1945674 |
| 28030 | 6: 2057409 | 28044 | 6: 2394123 | 28046 | 6: 2394185 | 28047 | 6: 2418816 | 28048 | 6: 2418834 |
| 28054 | 6: 2624789 | 28055 | 6: 2625544 | 28058 | 6: 2663078 | 28059 | 6: 2672214 | 28077 | 6: 3108815 |
| 28078 | 6: 3108854 | 28087 | 6: 3376371 | 28088 | 6: 3380776 | 28089 | 6: 3383123 | 28090 | 6: 3386603 |
| 28091 | 6: 3388251 | 28092 | 6: 3392342 | 28094 | 6: 3398350 | 28095 | 6: 3399218 | 28096 | 6: 3399858 |
| 28097 | 6: 3402165 | 28099 | 6: 3423360 | 28100 | 6: 3425355 | 28102 | 6: 3444538 | 28104 | 6: 3449326 |
| 28106 | 6: 3469199 | 28110 | 6: 3575704 | 28111 | 6: 3585145 | 28112 | 6: 3587923 | 28113 | 6: 3587933 |
| 28114 | 6: 3588243 | 28115 | 6: 3588261 | 28116 | 6: 3589475 | 28117 | 6: 3589994 | 28124 | 6: 3680340 |
| 28142 | 6: 4189994 | 28143 | 6: 4197131 | 28147 | 6: 4297910 | 28149 | 6: 4300166 | 28179 | 6: 4998395 |
| 28183 | 6: 5135323 | 28188 | 6: 5332029 | 28190 | 6: 5359913 | 28191 | 6: 5363670 | 28192 | 6: 5366571 |
| 28193 | 6: 5417516 | 28194 | 6: 5420554 | 28198 | 6: 5638260 | 28208 | 6: 5736468 | 28213 | 6: 5970478 |
| 28215 | 6: 6023720 | 28217 | 6: 6068366 | 28218 | 6: 6086349 | 28219 | 6: 6090367 | 28220 | 6: 6099226 |
| 28222 | 6: 6135400 | 28223 | 6: 6135571 | 28224 | 6: 6135977 | 28225 | 6: 6142753 | 28226 | 6: 6145385 |
| 28227 | 6: 6146291 | 28229 | 6: 6150122 | 28231 | 6: 6213823 | 28233 | 6: 6252521 | 28236 | 6: 6303620 |
| 28237 | 6: 6304251 | 28241 | 6: 6327370 | 28246 | 6: 6434692 | 28248 | 6: 6445175 | 28251 | 6: 6505947 |
| 28252 | 6: 6508802 | 28253 | 6: 6508845 | 28256 | 6: 6598639 | 28257 | 6: 6630222 | 28259 | 6: 6653707 |
| 28260 | 6: 6653733 | 28261 | 6: 6653820 | 28262 | 6: 6654166 | 28263 | 6: 6654264 | 28265 | 6: 6674156 |
| 28266 | 6: 6675058 | 28296 | 6: 7438091 | 28297 | 6: 7438117 | 28306 | 6: 7840060 | 28315 | 6: 8178961 |
| 28325 | 6: 8498664 | 28330 | 6: 8732227 | 28331 | 6: 8782400 | 28338 | 6: 8951172 | 28340 | 6: 8957062 |
| 28344 | 6: 8978291 | 28348 | 6: 8993893 | 28359 | 6: 9166519 | 28360 | 6: 9196740 | 28361 | 6: 9200024 |
| 28362 | 6: 9200854 | 28363 | 6: 9201774 | 28364 | 6: 9203527 | 28365 | 6: 9204459 | 28366 | 6: 9205411 |
| 28367 | 6: 9207235 | 28368 | 6: 9208280 | 28369 | 6: 9209206 | 28411 | 6: 10509217 | 28417 | 6: 10679784 |
| 28436 | 6: 11171281 | 28444 | 6: 11394489 | 28448 | 6: 11465975 | 28461 | 6: 11714410 | 28468 | 6: 11859669 |
| 28469 | 6: 11867154 | 28471 | 6: 11887100 | 28489 | 6: 12266091 | 28492 | 6: 12331408 | 28493 | 6: 12406590 |
| 28496 | 6: 12449553 | 28499 | 6: 12460758 | 28505 | 6: 12583026 | 28520 | 6: 12951400 | 28523 | 6: 13000409 |
| 28524 | 6: 13001855 | 28525 | 6: 13027415 | 28528 | 6: 13118294 | 28531 | 6: 13124831 | 28532 | 6: 13124859 |
| 28533 | 6: 13124885 | 28539 | 6: 13212064 | 28550 | 6: 13497569 | 28554 | 6: 13707108 | 28566 | 6: 14075733 |
| 28572 | 6: 14295399 | 28592 | 6: 14776571 | 28593 | 6: 14795242 | 28596 | 6: 14878264 | 28598 | 6: 14937683 |
| 28614 | 6: 15271409 | 28615 | 6: 15279031 | 28628 | 6: 15511470 | 28631 | 6: 15552360 | 28632 | 6: 15552836 |
| 28633 | 6: 15552929 | 28634 | 6: 15554381 | 28635 | 6: 15554470 | 28639 | 6: 15665547 | 28640 | 6: 15665565 |
| 28644 | 6: 15724964 | 28659 | 6: 16024226 | 28672 | 6: 16407876 | 28675 | 6: 16429738 | 28676 | 6: 16431811 |
| 28680 | 6: 16480595 | 28681 | 6: 16483148 | 28688 | 6: 16631779 | 28690 | 6: 16706078 | 28692 | 6: 16770941 |
| 28701 | 6: 16924337 | 28702 | 6: 16926868 | 28704 | 6: 16957756 | 28710 | 6: 17056311 | 28711 | 6: 17056315 |
| 28712 | 6: 17080889 | 28734 | 6: 17680046 | 28737 | 6: 17695292 | 28746 | 6: 17927268 | 28747 | 6: 17931957 |
| 28748 | 6: 17938149 | 28761 | 6: 18039717 | 28772 | 6: 18212765 | 28777 | 6: 18284502 | 28778 | 6: 18314365 |
| 28789 | 6: 18646895 | 28790 | 6: 18701849 | 28791 | 6: 18731749 | 28799 | 6: 18864835 | 28807 | 6: 19042586 |
| 28809 | 6: 19074411 | 28811 | 6: 19074485 | 28815 | 6: 19190465 | 28826 | 6: 19438867 | 28827 | 6: 19439848 |
| 28829 | 6: 19456967 | 28830 | 6: 19462913 | 28831 | 6: 19495081 | 28839 | 6: 19649377 | 28848 | 6: 19872402 |
| 28850 | 6: 19894311 | 28851 | 6: 19923403 | 28858 | 6: 20135992 | 28860 | 6: 20184822 | 28861 | 6: 20192872 |
| 28862 | 6: 20206522 | 28872 | 6: 20397821 | 28876 | 6: 20495916 | 28881 | 6: 20600966 | 28882 | 6: 20600990 |
| 28907 | 6: 21325769 | 28908 | 6: 21355207 | 28910 | 6: 21392641 | 28913 | 6: 21439632 | 28914 | 6: 21439691 |
| 28915 | 6: 21439763 | 28921 | 6: 21659954 | 28922 | 6: 21672266 | 28923 | 6: 21678873 | 28925 | 6: 21686069 |
| 28926 | 6: 21691325 | 28927 | 6: 21703290 | 28928 | 6: 21714263 | 28933 | 6: 21771563 | 28934 | 6: 21772453 |
| 28935 | 6: 21777544 | 28939 | 6: 21951701 | 28942 | 6: 22033622 | 28962 | 6: 22379732 | 28964 | 6: 22422233 |
| 28967 | 6: 22500214 | 28968 | 6: 22500233 | 28991 | 6: 23037086 | 28997 | 6: 23243928 | 29000 | 6: 23295269 |
| 29005 | 6: 23444102 | 29006 | 6: 23457488 | 29010 | 6: 23533175 | 29013 | 6: 23555860 | 29015 | 6: 23571729 |
| 29024 | 6: 23873913 | 29029 | 6: 23921917 | 29047 | 6: 24373068 | 29051 | 6: 24503230 | 29053 | 6: 24518463 |
| 29064 | 6: 24837558 | 29070 | 6: 24942515 | 29071 | 6: 24948898 | 29076 | 6: 25007714 | 29077 | 6: 25024841 |
| 29078 | 6: 25039222 | 29083 | 6: 25164226 | 29095 | 6: 25528726 | 29100 | 6: 25695588 | 29102 | 6: 25788477 |
| 29108 | 6: 25887098 | 29117 | 6: 26022919 | 29118 | 6: 26033719 | 29127 | 6: 26200013 | 29129 | 6: 26282778 |
| 29130 | 6: 26290561 | 29139 | 6: 26655531 | 29140 | 6: 26655687 | 29142 | 6: 26679658 | 29163 | 6: 27215526 |
| 29167 | 6: 27355784 | 29174 | 6: 27472557 | 29186 | 6: 27864310 | 29212 | 6: 28647979 | 29231 | 6: 29128064 |
| 29237 | 6: 29212166 | 29239 | 6: 29328156 | 29240 | 6: 29328334 | 29241 | 6: 29328350 | 29243 | 6: 29376641 |
| 29245 | 6: 29389036 | 29247 | 6: 29435290 | 29253 | 6: 29483476 | 29259 | 6: 29565346 | 29265 | 6: 29684500 |
| 29279 | 6: 30075635 | 29342 | 6: 30145040 | 29365 | 6: 30563336 | 29399 | 6: 31576983 | 29412 | 6: 31922816 |
| 29421 | 6: 32195477 | 29425 | 6: 32453198 | 29426 | 6: 32493429 | 29468 | 6: 34036502 | 29472 | 6: 34067879 |
| 29473 | 6: 34068521 | 29486 | 6: 34217305 | 29587 | 6: 34344002 | 29660 | 6: 34467878 | 29733 | 6: 34605510 |
| 29954 | 6: 35510819 | 29957 | 6: 35561016 | 29986 | 6: 35857940 | 30022 | 6: 36053886 | 30024 | 6: 36074911 |
| 30130 | 6: 36395104 | 30139 | 6: 36431315 | 30145 | 6: 36444505 | 30148 | 6: 36448732 | 30174 | 6: 36489959 |
| 30250 | 6: 36553066 | 30255 | 6: 36561452 | 30300 | 6: 36642083 | 30317 | 6: 36688082 | 30321 | 6: 36706370 |
| 30383 | 6: 36857123 | 30808 | 6: 37218423 | 31143 | 6: 37396789 | 31963 | 6: 39186781 | 31964 | 6: 39249570 |
| 31972 | 6: 39371669 | 31973 | 6: 39392848 | 31997 | 6: 39755061 | 32033 | 6: 40227846 | 32038 | 6: 40262465 |
| 32095 | 6: 40813933 | 32099 | 6: 40846711 | 32101 | 6: 40869544 | 32162 | 6: 41204988 | 32163 | 6: 41205018 |
| 32194 | 6: 41998863 | 32195 | 6: 42004652 | 32196 | 6: 42005216 | 32197 | 6: 42010829 | 32198 | 6: 42011964 |
| 32199 | 6: 42014380 | 32200 | 6: 42017579 | 32202 | 6: 42030911 | 32206 | 6: 42098249 | 32238 | 6: 42816146 |
| 32247 | 6: 42950720 | 32272 | 6: 43082880 | 32273 | 6: 43090582 | 32274 | 6: 43121398 | 32288 | 6: 43377340 |
| 32289 | 6: 43378488 | 32310 | 6: 43688837 | 32324 | 6: 43887416 | 32334 | 6: 44040805 | 32335 | 6: 44059941 |
| 32336 | 6: 44072335 | 32349 | 6: 44361206 | 32359 | 6: 44628166 | 32367 | 6: 44692016 | 32483 | 6: 45118837 |
| 32493 | 6: 45218126 | 32498 | 6: 45277318 | 32501 | 6: 45322457 | 32503 | 6: 45335602 | 32504 | 6: 45364598 |
| 32507 | 6: 45390455 | 32509 | 6: 45416952 | 32513 | 6: 45505416 | 32514 | 6: 45522021 | 32520 | 6: 45688164 |
| 32531 | 6: 45935668 | 32532 | 6: 45982939 | 32534 | 6: 46022376 | 32535 | 6: 46078755 | 32545 | 6: 46209779 |
| 32550 | 6: 46337526 | 32552 | 6: 46378480 | 32553 | 6: 46389387 | 32554 | 6: 46390042 | 32577 | 6: 46954860 |
| 32582 | 6: 47076276 | 32595 | 6: 47353083 | 32606 | 6: 47640345 | 32616 | 6: 47866852 | 32618 | 6: 47903812 |
| 32622 | 6: 48018241 | 32634 | 6: 48398260 | 32642 | 6: 48750204 | 32664 | 6: 49330544 | 32666 | 6: 49366546 |
| 32681 | 6: 49731100 | 32682 | 6: 49732769 | 32683 | 6: 49734879 | 32684 | 6: 49738480 | 32685 | 6: 49759413 |
| 32686 | 6: 49766766 | 32687 | 6: 49773026 | 32690 | 6: 49804879 | 32691 | 6: 49816719 | 32697 | 6: 49895642 |
| 32705 | 6: 50190150 | 32709 | 6: 50252554 | 32711 | 6: 50316194 | 32712 | 6: 50316384 | 32713 | 6: 50316707 |
| 32714 | 6: 50342873 | 32745 | 6: 51287866 | 32761 | 6: 51779722 | 32770 | 6: 52097852 | 32771 | 6: 52097941 |
| 32776 | 6: 52230394 | 32777 | 6: 52230455 | 32779 | 6: 52258121 | 32811 | 6: 52492036 | 32823 | 6: 52776675 |
| 32839 | 6: 53189333 | 32841 | 6: 53215914 | 32845 | 6: 53260563 | 32856 | 6: 53542273 | 32858 | 6: 53587223 |
| 32871 | 6: 53982975 | 32882 | 6: 54231133 | 32895 | 6: 54705767 | 32905 | 6: 55072307 | 32957 | 6: 55697219 |
| 32965 | 6: 55964167 | 32981 | 6: 56569534 | 32982 | 6: 56580948 | 32997 | 6: 56927864 | 32998 | 6: 56927881 |
| 32999 | 6: 56927920 | 33000 | 6: 56928100 | 33011 | 6: 57258298 | 33013 | 6: 57310043 | 33016 | 6: 57383139 |
| 33019 | 6: 57452741 | 33021 | 6: 57538068 | 33024 | 6: 57589915 | 33053 | 6: 58398195 | 33055 | 6: 58507178 |
| 33065 | 6: 58716078 | 33075 | 6: 59092959 | 33080 | 6: 59188156 | 33085 | 6: 59262762 | 33100 | 6: 59749739 |
| 33108 | 6: 59853909 | 33118 | 6: 59966318 | 33123 | 6: 60097949 | 33128 | 6: 60226786 | 33130 | 6: 60265173 |
| 33131 | 6: 60301627 | 33133 | 6: 60345603 | 33135 | 6: 60373758 | 33138 | 6: 60422003 | 33140 | 6: 60468668 |
| 33152 | 6: 60773644 | 33166 | 6: 61158905 | 33167 | 6: 61161272 | 33172 | 6: 61280689 | 33177 | 6: 61397201 |
| 33179 | 6: 61410508 | 33180 | 6: 61421440 | 33187 | 6: 61548278 | 33188 | 6: 61582228 | 33189 | 6: 61589076 |
| 33190 | 6: 61673425 | 33217 | 6: 62603953 | 33226 | 6: 62889998 | 33231 | 6: 62988117 | 33232 | 6: 63010364 |
| 33233 | 6: 63010380 | 33235 | 6: 63029593 | 33237 | 6: 63097017 | 33247 | 6: 63307538 | 33248 | 6: 63308671 |
| 33249 | 6: 63347756 | 33269 | 6: 64151594 | 33281 | 6: 64662329 | 33282 | 6: 64665326 | 33293 | 6: 65016085 |
| 33300 | 6: 65345032 | 33302 | 6: 65383049 | 33304 | 6: 65420404 | 33305 | 6: 65422497 | 33306 | 6: 65422592 |
| 33307 | 6: 65456807 | 33325 | 6: 66109765 | 33332 | 6: 66343250 | 33334 | 6: 66387388 | 33340 | 6: 66651619 |
| 33345 | 6: 66831939 | 33348 | 6: 66876573 | 33353 | 6: 66988792 | 33356 | 6: 67035579 | 33358 | 6: 67089021 |
| 33373 | 6: 67582129 | 33382 | 6: 68017233 | 33387 | 6: 68177405 | 33390 | 6: 68268998 | 33395 | 6: 68352179 |
| 33401 | 6: 68565298 | 33402 | 6: 68589087 | 33403 | 6: 68598341 | 33410 | 6: 68728215 | 33413 | 6: 68763773 |
| 33415 | 6: 68828972 | 33417 | 6: 68885563 | 33420 | 6: 68935879 | 33426 | 6: 69138647 | 33428 | 6: 69168309 |
| 33430 | 6: 69216815 | 33434 | 6: 69287580 | 33436 | 6: 69352586 | 33439 | 6: 69439499 | 33445 | 6: 69755083 |
| 33452 | 6: 69895326 | 33464 | 6: 70383634 | 33465 | 6: 70399966 | 33466 | 6: 70404486 | 33467 | 6: 70405959 |
| 33468 | 6: 70423245 | 33472 | 6: 70564450 | 33481 | 6: 70877482 | 33482 | 6: 70900974 | 33490 | 6: 71126939 |
| 33494 | 6: 71294876 | 33504 | 6: 71562156 | 33510 | 6: 71703879 | 33518 | 6: 72060315 | 33522 | 6: 72174384 |
| 33531 | 6: 72351300 | 33533 | 6: 72379846 | 33540 | 6: 72542846 | 33548 | 6: 72783452 | 33558 | 6: 73223485 |
| 33565 | 6: 73414475 | 33567 | 6: 73481332 | 33570 | 6: 73620191 | 33577 | 6: 73801067 | 33631 | 6: 75798192 |
| 33645 | 6: 76545846 | 33646 | 6: 76546983 | 33647 | 6: 76547862 | 33665 | 6: 76855629 | 33677 | 6: 77240842 |
| 33680 | 6: 77349471 | 33691 | 6: 77688509 | 33694 | 6: 77787079 | 33711 | 6: 78425706 | 33712 | 6: 78429257 |
| 33717 | 6: 78761621 | 33718 | 6: 78761714 | 33721 | 6: 78917750 | 33743 | 6: 79765417 | 33765 | 6: 80471442 |
| 33766 | 6: 80482749 | 33767 | 6: 80484712 | 33768 | 6: 80491703 | 33769 | 6: 80530130 | 33770 | 6: 80567928 |
| 33771 | 6: 80590178 | 33773 | 6: 80675168 | 33788 | 6: 81051057 | 33814 | 6: 82117911 | 33855 | 6: 83277672 |
| 33858 | 6: 83308141 | 33861 | 6: 83347263 | 33864 | 6: 83451575 | 33865 | 6: 83451628 | 33866 | 6: 83451712 |
| 33867 | 6: 83451781 | 33868 | 6: 83451862 | 33870 | 6: 83469477 | 33871 | 6: 83470850 | 33872 | 6: 83475021 |
| 33884 | 6: 83758356 | 33885 | 6: 83763398 | 33892 | 6: 83946894 | 33893 | 6: 83948279 | 33894 | 6: 83950501 |
| 33898 | 6: 83985933 | 33902 | 6: 84087846 | 33903 | 6: 84138032 | 33910 | 6: 84497175 | 33911 | 6: 84497631 |
| 33912 | 6: 84497709 | 33913 | 6: 84511587 | 33914 | 6: 84511656 | 33915 | 6: 84513009 | 33932 | 6: 84975132 |
| 33933 | 6: 85067620 | 33934 | 6: 85092477 | 33935 | 6: 85092499 | 33936 | 6: 85092523 | 33937 | 6: 85093071 |
| 33938 | 6: 85108153 | 33939 | 6: 85108165 | 33940 | 6: 85108432 | 33941 | 6: 85108773 | 33943 | 6: 85136420 |
| 33944 | 6: 85137180 | 33950 | 6: 85315912 | 33977 | 6: 85896040 | 33978 | 6: 85896097 | 33979 | 6: 85896105 |
| 33980 | 6: 85896139 | 33981 | 6: 85906390 | 33982 | 6: 85908497 | 34003 | 6: 86508590 | 34012 | 6: 86710155 |
| 34028 | 6: 86901499 | 34032 | 6: 86989414 | 34034 | 6: 87007077 | 34040 | 6: 87076603 | 34043 | 6: 87111828 |
| 34057 | 6: 87463348 | 34067 | 6: 87603473 | 34074 | 6: 87731662 | 34082 | 6: 87796536 | 34085 | 6: 87923433 |
| 34086 | 6: 87923536 | 34092 | 6: 88075544 | 34096 | 6: 88164435 | 34097 | 6: 88184411 | 34098 | 6: 88184633 |
| 34102 | 6: 88318314 | 34108 | 6: 88486850 | 34116 | 6: 88859149 | 34130 | 6: 89152164 | 34131 | 6: 89181036 |
| 34150 | 6: 89663234 | 34152 | 6: 89663319 | 34153 | 6: 89663348 | 34154 | 6: 89674618 | 34162 | 6: 89768711 |
| 34170 | 6: 89975431 | 34182 | 6: 90390426 | 34185 | 6: 90416932 | 34186 | 6: 90457703 | 34196 | 6: 90680946 |
| 34199 | 6: 90714640 | 34200 | 6: 90726127 | 34206 | 6: 90794035 | 34208 | 6: 90816980 | 34210 | 6: 90823124 |
| 34211 | 6: 90826546 | 34212 | 6: 90826598 | 34213 | 6: 90827159 | 34214 | 6: 90836066 | 34215 | 6: 90836072 |
| 34216 | 6: 90836166 | 34221 | 6: 90946704 | 34228 | 6: 91105837 | 34229 | 6: 91122456 | 34236 | 6: 91204532 |
| 34237 | 6: 91223593 | 34238 | 6: 91226875 | 34239 | 6: 91246239 | 34240 | 6: 91275706 | 34241 | 6: 91282834 |
| 34242 | 6: 91284101 | 34243 | 6: 91284414 | 34244 | 6: 91285011 | 34245 | 6: 91285694 | 34246 | 6: 91285851 |
| 34247 | 6: 91285925 | 34248 | 6: 91290335 | 34249 | 6: 91294383 | 34250 | 6: 91322998 | 34257 | 6: 91406353 |
| 34258 | 6: 91408596 | 34260 | 6: 91433962 | 34261 | 6: 91462732 | 34271 | 6: 91615288 | 34272 | 6: 91616087 |
| 34275 | 6: 91649638 | 34277 | 6: 91692231 | 34280 | 6: 91736404 | 34282 | 6: 91751612 | 34284 | 6: 91836628 |
| 34287 | 6: 91878223 | 34291 | 6: 91994872 | 34300 | 6: 92336028 | 34302 | 6: 92383295 | 34308 | 6: 92595572 |
| 34310 | 6: 92678382 | 34313 | 6: 92774897 | 34317 | 6: 92832688 | 34335 | 6: 93291707 | 34336 | 6: 93291712 |
| 34337 | 6: 93291776 | 34343 | 6: 93354640 | 34344 | 6: 93363500 | 34351 | 6: 93496619 | 34353 | 6: 93533733 |
| 34361 | 6: 93646233 | 34373 | 6: 93947051 | 34378 | 6: 94069815 | 34389 | 6: 94150648 | 34392 | 6: 94188032 |
| 34393 | 6: 94201168 | 34394 | 6: 94204543 | 34395 | 6: 94215369 | 34401 | 6: 94394468 | 34403 | 6: 94416755 |
| 34413 | 6: 94854202 | 34424 | 6: 95339487 | 34426 | 6: 95406474 | 34427 | 6: 95406527 | 34429 | 6: 95406556 |
| 34432 | 6: 95553456 | 34441 | 6: 95787499 | 34444 | 6: 95884565 | 34449 | 6: 96032153 | 34451 | 6: 96087038 |
| 34462 | 6: 96247005 | 34474 | 6: 96452752 | 34496 | 6: 97018222 | 34504 | 6: 97179175 | 34505 | 6: 97179176 |
| 34506 | 6: 97179179 | 34507 | 6: 97179203 | 34508 | 6: 97179214 | 34509 | 6: 97179248 | 34510 | 6: 97217522 |
| 34511 | 6: 97226901 | 34512 | 6: 97244565 | 34513 | 6: 97244652 | 34514 | 6: 97244662 | 34515 | 6: 97255320 |
| 34516 | 6: 97255455 | 34517 | 6: 97255461 | 34528 | 6: 97447881 | 34529 | 6: 97449063 | 34530 | 6: 97453689 |
| 34544 | 6: 97650137 | 34547 | 6: 97742989 | 34556 | 6: 98047323 | 34558 | 6: 98052606 | 34566 | 6: 98256336 |
| 34570 | 6: 98334890 | 34571 | 6: 98337762 | 34572 | 6: 98337909 | 34573 | 6: 98337919 | 34574 | 6: 98338489 |
| 34575 | 6: 98338596 | 34576 | 6: 98338640 | 34577 | 6: 98338644 | 34579 | 6: 98372826 | 34581 | 6: 98379884 |
| 34596 | 6: 98671599 | 34608 | 6: 98974175 | 34628 | 6: 99529167 | 34637 | 6: 99783201 | 34641 | 6: 99954538 |
| 34669 | 6: 100390632 | 34671 | 6: 100431779 | 34678 | 6: 100585876 | 34685 | 6: 100727406 | 34689 | 6: 100852199 |
| 34693 | 6: 100953221 | 34706 | 6: 101294567 | 34707 | 6: 101294597 | 34714 | 6: 101450871 | 34729 | 6: 101787815 |
| 34730 | 6: 101837470 | 34739 | 6: 101917014 | 34740 | 6: 101935968 | 34741 | 6: 101941367 | 34744 | 6: 101973857 |
| 34754 | 6: 102148393 | 34755 | 6: 102148443 | 34760 | 6: 102234263 | 34766 | 6: 102424852 | 34772 | 6: 102510343 |
| 34779 | 6: 102606444 | 34783 | 6: 102645168 | 34784 | 6: 102645192 | 34785 | 6: 102645198 | 34788 | 6: 102756289 |
| 34791 | 6: 102773226 | 34793 | 6: 102854233 | 34795 | 6: 102855258 | 34796 | 6: 102857313 | 34797 | 6: 102870507 |
| 34798 | 6: 102871524 | 34799 | 6: 102872128 | 34800 | 6: 102873595 | 34801 | 6: 102875781 | 34802 | 6: 102889726 |
| 34803 | 6: 102890450 | 34804 | 6: 102892630 | 34805 | 6: 102896127 | 34806 | 6: 102896985 | 34807 | 6: 102927406 |
| 34812 | 6: 103013214 | 34822 | 6: 103212156 | 34835 | 6: 103357734 | 34837 | 6: 103396597 | 34839 | 6: 103431771 |
| 34841 | 6: 103480885 | 34842 | 6: 103490310 | 34843 | 6: 103490339 | 34844 | 6: 103490470 | 34848 | 6: 103557818 |
| 34851 | 6: 103637428 | 34852 | 6: 103640406 | 34854 | 6: 103654616 | 34855 | 6: 103663732 | 34856 | 6: 103741666 |
| 34857 | 6: 103773464 | 34858 | 6: 103774096 | 34862 | 6: 103862052 | 34864 | 6: 103898267 | 34876 | 6: 104267666 |
| 34881 | 6: 104339504 | 34892 | 6: 104657713 | 34893 | 6: 104658492 | 34897 | 6: 104793010 | 34898 | 6: 104793414 |
| 34899 | 6: 104809492 | 34900 | 6: 104809647 | 34901 | 6: 104824268 | 34903 | 6: 104868243 | 34905 | 6: 104881408 |
| 34909 | 6: 104928796 | 34914 | 6: 105104413 | 34924 | 6: 105290637 | 34927 | 6: 105378109 | 34929 | 6: 105423924 |
| 34931 | 6: 105504062 | 34940 | 6: 105638984 | 34941 | 6: 105650631 | 34950 | 6: 105886896 | 34959 | 6: 106123065 |
| 34966 | 6: 106312210 | 34971 | 6: 106540140 | 34979 | 6: 106739419 | 34986 | 6: 106953368 | 35009 | 6: 107508591 |
| 35024 | 6: 108020924 | 35045 | 6: 108596491 | 35055 | 6: 108951951 | 35057 | 6: 108952102 | 35059 | 6: 108993006 |
| 35068 | 6: 109199682 | 35071 | 6: 109282471 | 35072 | 6: 109282486 | 35102 | 6: 109823696 | 35111 | 6: 110018683 |
| 35142 | 6: 110852081 | 35156 | 6: 111034643 | 35157 | 6: 111034669 | 35160 | 6: 111043273 | 35167 | 6: 111198616 |
| 35176 | 6: 111399485 | 35180 | 6: 111474948 | 35189 | 6: 111673792 | 35198 | 6: 111776075 | 35200 | 6: 111795733 |
| 35201 | 6: 111796366 | 35205 | 6: 111879814 | 35206 | 6: 111880060 | 35208 | 6: 111944736 | 35209 | 6: 111951572 |
| 35226 | 6: 112530579 | 35231 | 6: 112650810 | 35234 | 6: 112672985 | 35239 | 6: 112735477 | 35241 | 6: 112750366 |
| 35247 | 6: 112863762 | 35251 | 6: 112908010 | 35255 | 6: 112989544 | 35262 | 6: 113261157 | 35272 | 6: 113442820 |
| 35276 | 6: 113471367 | 35277 | 6: 113480954 | 35281 | 6: 113591450 | 35288 | 6: 113702935 | 35290 | 6: 113754589 |
| 35291 | 6: 113756287 | 35292 | 6: 113756982 | 35293 | 6: 113765934 | 35295 | 6: 113775951 | 35297 | 6: 113799562 |
| 35298 | 6: 113821699 | 35300 | 6: 113827124 | 35301 | 6: 113830450 | 35309 | 6: 113999468 | 35311 | 6: 114031327 |
| 35312 | 6: 114040384 | 35320 | 6: 114163874 | 35324 | 6: 114205337 | 35327 | 6: 114271753 | 35334 | 6: 114428994 |
| 35336 | 6: 114452241 | 35337 | 6: 114471056 | 35342 | 6: 114583005 | 35344 | 6: 114601159 | 35345 | 6: 114602046 |
| 35351 | 6: 114719008 | 35355 | 6: 114797024 | 35365 | 6: 115032289 | 35366 | 6: 115035590 | 35367 | 6: 115035592 |
| 35376 | 6: 115201677 | 35382 | 6: 115369888 | 35401 | 6: 115856852 | 35403 | 6: 115872479 | 35404 | 6: 115959886 |
| 35408 | 6: 116004094 | 35409 | 6: 116011104 | 35416 | 6: 116154808 | 35417 | 6: 116160046 | 35420 | 6: 116200574 |
| 35422 | 6: 116286341 | 35425 | 6: 116319165 | 35430 | 6: 116485262 | 35431 | 6: 116485283 | 35432 | 6: 116485335 |
| 35433 | 6: 116485360 | 35434 | 6: 116485372 | 35435 | 6: 116485909 | 35436 | 6: 116487900 | 35437 | 6: 116487922 |
| 35438 | 6: 116488922 | 35439 | 6: 116489686 | 35440 | 6: 116489801 | 35441 | 6: 116500010 | 35442 | 6: 116500063 |
| 35443 | 6: 116500364 | 35444 | 6: 116500379 | 35445 | 6: 116509870 | 35446 | 6: 116514508 | 35447 | 6: 116514529 |
| 35453 | 6: 116932235 | 35457 | 6: 117212852 | 35458 | 6: 117247186 | 35459 | 6: 117253860 | 35462 | 6: 117399949 |
| 35463 | 6: 117405411 | 35464 | 6: 117444202 | 35465 | 6: 117445801 | 35466 | 6: 117445935 | 35467 | 6: 117463206 |
| 35490 | 7: 516102 | 35502 | 7: 810744 | 35503 | 7: 822079 | 35533 | 7: 1434030 | 35534 | 7: 1439391 |
| 35535 | 7: 1444418 | 35537 | 7: 1508351 | 35539 | 7: 1552952 | 35546 | 7: 1663078 | 35547 | 7: 1663134 |
| 35550 | 7: 1706720 | 35553 | 7: 1757821 | 35560 | 7: 1977089 | 35562 | 7: 1996339 | 35563 | 7: 2036084 |
| 35567 | 7: 2112952 | 35577 | 7: 2352038 | 35587 | 7: 2461961 | 35590 | 7: 2556375 | 35594 | 7: 2628007 |
| 35595 | 7: 2628008 | 35596 | 7: 2628014 | 35597 | 7: 2628018 | 35598 | 7: 2628066 | 35599 | 7: 2628069 |
| 35600 | 7: 2628233 | 35601 | 7: 2628255 | 35602 | 7: 2628256 | 35604 | 7: 2630943 | 35605 | 7: 2630947 |
| 35606 | 7: 2631054 | 35607 | 7: 2631064 | 35610 | 7: 2682673 | 35611 | 7: 2682716 | 35612 | 7: 2682743 |
| 35613 | 7: 2682770 | 35614 | 7: 2682781 | 35615 | 7: 2682785 | 35618 | 7: 2721521 | 35619 | 7: 2728053 |
| 35622 | 7: 2823155 | 35624 | 7: 2880427 | 35629 | 7: 2957250 | 35631 | 7: 3004323 | 35641 | 7: 3142936 |
| 35643 | 7: 3239293 | 35670 | 7: 3679709 | 35671 | 7: 3679809 | 35672 | 7: 3686101 | 35673 | 7: 3687496 |
| 35683 | 7: 3876358 | 35687 | 7: 3947888 | 35688 | 7: 3949113 | 35689 | 7: 3953770 | 35690 | 7: 3965550 |
| 35692 | 7: 4018660 | 35693 | 7: 4057873 | 35700 | 7: 4183821 | 35702 | 7: 4203677 | 35704 | 7: 4226758 |
| 35706 | 7: 4321618 | 35708 | 7: 4425609 | 35709 | 7: 4432045 | 35713 | 7: 4509941 | 35714 | 7: 4530497 |
| 35715 | 7: 4550094 | 35724 | 7: 4890795 | 35725 | 7: 4931147 | 35733 | 7: 5056411 | 35734 | 7: 5076528 |
| 35735 | 7: 5077856 | 35736 | 7: 5077899 | 35737 | 7: 5077986 | 35738 | 7: 5078073 | 35743 | 7: 5135884 |
| 35744 | 7: 5135971 | 35747 | 7: 5227598 | 35758 | 7: 5539362 | 35759 | 7: 5540415 | 35760 | 7: 5541785 |
| 35761 | 7: 5542691 | 35762 | 7: 5543242 | 35763 | 7: 5543903 | 35764 | 7: 5544868 | 35765 | 7: 5545851 |
| 35766 | 7: 5546627 | 35767 | 7: 5547659 | 35768 | 7: 5548421 | 35769 | 7: 5554183 | 35770 | 7: 5554705 |
| 35771 | 7: 5555278 | 35772 | 7: 5556329 | 35773 | 7: 5560431 | 35775 | 7: 5587078 | 35781 | 7: 5670138 |
| 35783 | 7: 5725687 | 35784 | 7: 5728200 | 35787 | 7: 5774079 | 35788 | 7: 5775685 | 35789 | 7: 5775738 |
| 35790 | 7: 5775833 | 35791 | 7: 5775864 | 35794 | 7: 5854438 | 35795 | 7: 5867893 | 35796 | 7: 5869641 |
| 35797 | 7: 5875629 | 35798 | 7: 5876835 | 35799 | 7: 5879220 | 35800 | 7: 5884897 | 35801 | 7: 5888476 |
| 35803 | 7: 5895045 | 35804 | 7: 5905064 | 35807 | 7: 5948030 | 35839 | 7: 6133269 | 35848 | 7: 6256347 |
| 35850 | 7: 6276752 | 35852 | 7: 6318895 | 35853 | 7: 6318915 | 35867 | 7: 6376467 | 35868 | 7: 6389582 |
| 35877 | 7: 6654578 | 35885 | 7: 6739561 | 35892 | 7: 6820748 | 35896 | 7: 6837253 | 35905 | 7: 7044892 |
| 35906 | 7: 7044893 | 35909 | 7: 7081346 | 35912 | 7: 7131477 | 35913 | 7: 7131577 | 35979 | 7: 9765475 |
| 35998 | 7: 10641237 | 36009 | 7: 11092130 | 36015 | 7: 11145935 | 36016 | 7: 11154819 | 36019 | 7: 11233180 |
| 36020 | 7: 11233207 | 36021 | 7: 11237049 | 36022 | 7: 11296724 | 36023 | 7: 11306644 | 36024 | 7: 11332158 |
| 36026 | 7: 11394630 | 36027 | 7: 11394736 | 36028 | 7: 11403649 | 36042 | 7: 11870658 | 36043 | 7: 11885250 |
| 36062 | 7: 12227428 | 36070 | 7: 12344743 | 36071 | 7: 12344879 | 36082 | 7: 12522109 | 36088 | 7: 12587487 |
| 36093 | 7: 12738904 | 36096 | 7: 12762542 | 36097 | 7: 12763942 | 36099 | 7: 12806635 | 36100 | 7: 12816543 |
| 36102 | 7: 12824241 | 36103 | 7: 12852855 | 36106 | 7: 12896445 | 36107 | 7: 12897971 | 36108 | 7: 12898039 |
| 36109 | 7: 12898062 | 36111 | 7: 12974218 | 36112 | 7: 12974361 | 36115 | 7: 13016195 | 36121 | 7: 13189985 |
| 36122 | 7: 13201701 | 36123 | 7: 13204223 | 36124 | 7: 13204306 | 36125 | 7: 13209001 | 36126 | 7: 13209008 |
| 36127 | 7: 13209051 | 36128 | 7: 13209093 | 36129 | 7: 13211564 | 36130 | 7: 13211698 | 36131 | 7: 13211746 |
| 36133 | 7: 13234671 | 36135 | 7: 13241201 | 36136 | 7: 13248867 | 36137 | 7: 13252997 | 36145 | 7: 13436247 |
| 36160 | 7: 13763740 | 36161 | 7: 13764174 | 36163 | 7: 13826339 | 36164 | 7: 13826607 | 36165 | 7: 13847719 |
| 36166 | 7: 13847770 | 36167 | 7: 13848168 | 36169 | 7: 13907560 | 36170 | 7: 13907747 | 36171 | 7: 13908190 |
| 36175 | 7: 13970540 | 36176 | 7: 13970551 | 36177 | 7: 13971039 | 36182 | 7: 14105363 | 36187 | 7: 14216180 |
| 36188 | 7: 14278452 | 36196 | 7: 14568342 | 36197 | 7: 14569870 | 36198 | 7: 14569910 | 36199 | 7: 14570835 |
| 36200 | 7: 14571116 | 36201 | 7: 14571263 | 36202 | 7: 14572010 | 36203 | 7: 14572312 | 36204 | 7: 14574790 |
| 36205 | 7: 14574794 | 36206 | 7: 14574824 | 36207 | 7: 14574906 | 36208 | 7: 14575332 | 36209 | 7: 14575373 |
| 36210 | 7: 14576408 | 36211 | 7: 14576429 | 36212 | 7: 14576444 | 36213 | 7: 14577168 | 36214 | 7: 14577625 |
| 36215 | 7: 14578417 | 36216 | 7: 14578461 | 36217 | 7: 14578495 | 36218 | 7: 14587075 | 36221 | 7: 14674502 |
| 36222 | 7: 14674589 | 36224 | 7: 14688808 | 36225 | 7: 14689037 | 36226 | 7: 14690346 | 36227 | 7: 14690813 |
| 36228 | 7: 14692758 | 36229 | 7: 14695086 | 36230 | 7: 14790790 | 36231 | 7: 14846055 | 36232 | 7: 14847442 |
| 36241 | 7: 15055421 | 36242 | 7: 15055460 | 36246 | 7: 15113333 | 36247 | 7: 15116085 | 36248 | 7: 15116605 |
| 36249 | 7: 15120550 | 36250 | 7: 15121347 | 36251 | 7: 15121459 | 36252 | 7: 15131143 | 36254 | 7: 15149591 |
| 36255 | 7: 15158037 | 36257 | 7: 15188314 | 36258 | 7: 15219440 | 36262 | 7: 15240130 | 36267 | 7: 15342910 |
| 36269 | 7: 15386114 | 36273 | 7: 15463697 | 36274 | 7: 15501033 | 36283 | 7: 15718347 | 36297 | 7: 16146481 |
| 36298 | 7: 16189628 | 36300 | 7: 16237783 | 36305 | 7: 16292747 | 36313 | 7: 16521674 | 36314 | 7: 16522621 |
| 36315 | 7: 16524699 | 36317 | 7: 16531552 | 36318 | 7: 16534211 | 36319 | 7: 16535641 | 36323 | 7: 16561541 |
| 36359 | 7: 17538355 | 36366 | 7: 17643702 | 36367 | 7: 17694868 | 36368 | 7: 17698407 | 36369 | 7: 17698410 |
| 36371 | 7: 17712921 | 36389 | 7: 18292860 | 36395 | 7: 18386812 | 36397 | 7: 18404440 | 36398 | 7: 18434206 |
| 36399 | 7: 18443286 | 36401 | 7: 18446615 | 36402 | 7: 18464757 | 36408 | 7: 18658963 | 36411 | 7: 18704573 |
| 36412 | 7: 18706088 | 36413 | 7: 18777233 | 36414 | 7: 18826386 | 36417 | 7: 18862062 | 36424 | 7: 19108968 |
| 36425 | 7: 19113510 | 36426 | 7: 19115360 | 36428 | 7: 19141191 | 36431 | 7: 19189844 | 36436 | 7: 19259287 |
| 36437 | 7: 19261276 | 36438 | 7: 19270709 | 36439 | 7: 19272732 | 36443 | 7: 19317767 | 36446 | 7: 19337660 |
| 36449 | 7: 19501368 | 36451 | 7: 19560991 | 36452 | 7: 19570534 | 36453 | 7: 19601934 | 36454 | 7: 19624357 |
| 36455 | 7: 19629075 | 36456 | 7: 19634135 | 36457 | 7: 19674365 | 36459 | 7: 19694033 | 36460 | 7: 19709157 |
| 36462 | 7: 19723388 | 36463 | 7: 19742259 | 36464 | 7: 19745894 | 36465 | 7: 19749906 | 36468 | 7: 19770884 |
| 36469 | 7: 19782434 | 36470 | 7: 19795534 | 36471 | 7: 19811128 | 36473 | 7: 19832477 | 36474 | 7: 19846412 |
| 36475 | 7: 19854110 | 36476 | 7: 19861073 | 36477 | 7: 19870302 | 36478 | 7: 19874067 | 36479 | 7: 19877997 |
| 36480 | 7: 19882846 | 36481 | 7: 19900064 | 36482 | 7: 19904145 | 36483 | 7: 19920619 | 36484 | 7: 19925859 |
| 36485 | 7: 19929090 | 36486 | 7: 19934553 | 36487 | 7: 19940802 | 36488 | 7: 19948369 | 36489 | 7: 19954948 |
| 36490 | 7: 19962302 | 36491 | 7: 19974241 | 36492 | 7: 19977349 | 36494 | 7: 20018689 | 36495 | 7: 20036738 |
| 36496 | 7: 20053188 | 36497 | 7: 20057534 | 36498 | 7: 20076160 | 36507 | 7: 20236317 | 36512 | 7: 20288732 |
| 36513 | 7: 20318995 | 36514 | 7: 20324208 | 36515 | 7: 20324611 | 36517 | 7: 20355414 | 36518 | 7: 20356311 |
| 36519 | 7: 20356363 | 36520 | 7: 20356383 | 36521 | 7: 20366443 | 36522 | 7: 20366466 | 36531 | 7: 20582777 |
| 36532 | 7: 20591858 | 36534 | 7: 20634478 | 36541 | 7: 20805581 | 36543 | 7: 20890886 | 36544 | 7: 20890946 |
| 36547 | 7: 20913247 | 36549 | 7: 20940338 | 36558 | 7: 21139922 | 36565 | 7: 21283821 | 36570 | 7: 21380240 |
| 36571 | 7: 21386399 | 36573 | 7: 21403549 | 36596 | 7: 21986701 | 36601 | 7: 22039959 | 36613 | 7: 22387715 |
| 36624 | 7: 22566377 | 36625 | 7: 22566448 | 36627 | 7: 22594692 | 36628 | 7: 22594727 | 36633 | 7: 22680039 |
| 36635 | 7: 22786294 | 36637 | 7: 22926260 | 36645 | 7: 23219383 | 36648 | 7: 23248102 | 36655 | 7: 23451699 |
| 36702 | 7: 24500311 | 36711 | 7: 24783223 | 36714 | 7: 24910871 | 36745 | 7: 25616363 | 36749 | 7: 25654878 |
| 36752 | 7: 25731016 | 36753 | 7: 25768089 | 36757 | 7: 25800631 | 36758 | 7: 25832593 | 36764 | 7: 25936782 |
| 36766 | 7: 26024639 | 36777 | 7: 26292161 | 36778 | 7: 26308990 | 36779 | 7: 26319916 | 36782 | 7: 26387075 |
| 36784 | 7: 26402172 | 36788 | 7: 26497485 | 36790 | 7: 26557163 | 36791 | 7: 26574444 | 36792 | 7: 26577081 |
| 36793 | 7: 26577087 | 36794 | 7: 26578715 | 36796 | 7: 26594068 | 36797 | 7: 26594073 | 36798 | 7: 26594090 |
| 36801 | 7: 26638130 | 36805 | 7: 26769987 | 36806 | 7: 26769998 | 36807 | 7: 26770016 | 36811 | 7: 26823826 |
| 36814 | 7: 26881081 | 36815 | 7: 26898868 | 36821 | 7: 27031629 | 36825 | 7: 27060226 | 36832 | 7: 27198824 |
| 36840 | 7: 27423246 | 36841 | 7: 27434826 | 36848 | 7: 27596371 | 36855 | 7: 27733288 | 36862 | 7: 27870224 |
| 36865 | 7: 28003332 | 36867 | 7: 28049997 | 36868 | 7: 28050030 | 36869 | 7: 28050091 | 36870 | 7: 28050098 |
| 36886 | 7: 28544979 | 36893 | 7: 28690143 | 36895 | 7: 28700180 | 36897 | 7: 28729276 | 36900 | 7: 28830783 |
| 36902 | 7: 28879641 | 36908 | 7: 29036081 | 36912 | 7: 29112331 | 36913 | 7: 29178982 | 36922 | 7: 29366546 |
| 36924 | 7: 29452732 | 36934 | 7: 29594179 | 36936 | 7: 29632089 | 36938 | 7: 29666350 | 36953 | 7: 29976577 |
| 36956 | 7: 30094509 | 36968 | 7: 30358105 | 36971 | 7: 30403065 | 36974 | 7: 30553468 | 36977 | 7: 30644004 |
| 36981 | 7: 30714382 | 36982 | 7: 30714446 | 36983 | 7: 30756401 | 36984 | 7: 30807818 | 36988 | 7: 30863698 |
| 36992 | 7: 30906898 | 36995 | 7: 30991586 | 36998 | 7: 31107264 | 37025 | 7: 31626205 | 37029 | 7: 31642354 |
| 37030 | 7: 31642496 | 37031 | 7: 31643684 | 37032 | 7: 31643707 | 37037 | 7: 31676529 | 37039 | 7: 31699035 |
| 37065 | 7: 32314617 | 37080 | 7: 32688342 | 37081 | 7: 32709556 | 37085 | 7: 32796768 | 37096 | 7: 33201093 |
| 37097 | 7: 33201168 | 37119 | 7: 33724498 | 37122 | 7: 33761341 | 37123 | 7: 33814744 | 37124 | 7: 33814750 |
| 37125 | 7: 33814773 | 37126 | 7: 33814812 | 37129 | 7: 33866953 | 37153 | 7: 34486136 | 37154 | 7: 34502493 |
| 37163 | 7: 34781679 | 37170 | 7: 34951198 | 37172 | 7: 34967857 | 37173 | 7: 34967898 | 37176 | 7: 35028517 |
| 37182 | 7: 35229836 | 37199 | 7: 35718668 | 37219 | 7: 36283231 | 37220 | 7: 36308740 | 37231 | 7: 36510620 |
| 37238 | 7: 36626658 | 37239 | 7: 36666622 | 37265 | 7: 37451557 | 37266 | 7: 37459216 | 37269 | 7: 37490139 |
| 37273 | 7: 37534315 | 37277 | 7: 37590371 | 37281 | 7: 37620741 | 37287 | 7: 37840929 | 37291 | 7: 37934995 |
| 37297 | 7: 38048904 | 37298 | 7: 38081521 | 37299 | 7: 38087100 | 37300 | 7: 38093801 | 37301 | 7: 38101864 |
| 37302 | 7: 38124565 | 37303 | 7: 38128610 | 37304 | 7: 38129977 | 37314 | 7: 38325007 | 37316 | 7: 38360533 |
| 37317 | 7: 38365253 | 37318 | 7: 38389751 | 37321 | 7: 38404339 | 37322 | 7: 38421353 | 37323 | 7: 38429558 |
| 37327 | 7: 38515000 | 37328 | 7: 38534271 | 37333 | 7: 38753839 | 37338 | 7: 38815021 | 37343 | 7: 38952074 |
| 37355 | 7: 39351372 | 37356 | 7: 39362501 | 37359 | 7: 39455426 | 37368 | 7: 39695685 | 37372 | 7: 39721571 |
| 37373 | 7: 39721626 | 37381 | 7: 40016354 | 37385 | 7: 40095095 | 37388 | 7: 40114076 | 37389 | 7: 40149307 |
| 37390 | 7: 40151612 | 37396 | 7: 40294613 | 37397 | 7: 40294728 | 37398 | 7: 40294729 | 37399 | 7: 40294731 |
| 37400 | 7: 40294744 | 37401 | 7: 40294745 | 37404 | 7: 40323542 | 37407 | 7: 40355854 | 37408 | 7: 40360599 |
| 37409 | 7: 40361142 | 37410 | 7: 40361184 | 37411 | 7: 40361244 | 37412 | 7: 40361336 | 37413 | 7: 40372170 |
| 37414 | 7: 40372221 | 37415 | 7: 40385848 | 37416 | 7: 40441629 | 37419 | 7: 40507729 | 37420 | 7: 40508207 |
| 37423 | 7: 40603077 | 37424 | 7: 40603099 | 37425 | 7: 40604704 | 37426 | 7: 40605637 | 37427 | 7: 40606059 |
| 37429 | 7: 40622112 | 37436 | 7: 40917929 | 37438 | 7: 40932803 | 37439 | 7: 40933182 | 37440 | 7: 40933299 |
| 37441 | 7: 40945845 | 37442 | 7: 40946121 | 37443 | 7: 40946770 | 37444 | 7: 40957931 | 37446 | 7: 40958025 |
| 37447 | 7: 40960932 | 37453 | 7: 41290614 | 37454 | 7: 41322935 | 37461 | 7: 41552605 | 37462 | 7: 41552746 |
| 37463 | 7: 41553267 | 37464 | 7: 41553442 | 37467 | 7: 41694074 | 37468 | 7: 41694108 | 37470 | 7: 41791738 |
| 37472 | 7: 41828300 | 37473 | 7: 41828310 | 37474 | 7: 41828476 | 37475 | 7: 41828489 | 37476 | 7: 41828529 |
| 37477 | 7: 41828531 | 37478 | 7: 41828618 | 37483 | 7: 42132719 | 37495 | 7: 42627355 | 37496 | 7: 42628923 |
| 37498 | 7: 42659874 | 37499 | 7: 42659928 | 37509 | 7: 43070478 | 37510 | 7: 43070495 | 37511 | 7: 43073489 |
| 37512 | 7: 43074460 | 37513 | 7: 43074485 | 37514 | 7: 43075276 | 37515 | 7: 43075327 | 37516 | 7: 43075329 |
| 37517 | 7: 43075823 | 37518 | 7: 43075911 | 37519 | 7: 43077087 | 37522 | 7: 43114270 | 37525 | 7: 43247821 |
| 37526 | 7: 43258641 | 37531 | 7: 43495660 | 37532 | 7: 43504923 | 37533 | 7: 43504924 | 37534 | 7: 43509552 |
| 37535 | 7: 43518518 | 37536 | 7: 43526310 | 37537 | 7: 43541646 | 37539 | 7: 43547038 | 37540 | 7: 43551313 |
| 37541 | 7: 43573739 | 37542 | 7: 43602807 | 37543 | 7: 43616877 | 37546 | 7: 43695498 | 37548 | 7: 43809877 |
| 37549 | 7: 43811263 | 37550 | 7: 43812894 | 37552 | 7: 43875604 | 37558 | 7: 44162642 | 37563 | 7: 44416303 |
| 37566 | 7: 44610929 | 37568 | 7: 44639553 | 37569 | 7: 44666191 | 37572 | 7: 44764045 | 37573 | 7: 44764061 |
| 37574 | 7: 44789031 | 37575 | 7: 44789067 | 37576 | 7: 44789088 | 37584 | 7: 44943643 | 37585 | 7: 45003392 |
| 37591 | 7: 45101749 | 37599 | 7: 45350646 | 37600 | 7: 45355058 | 37605 | 7: 45445789 | 37614 | 7: 45661047 |
| 37619 | 7: 45951627 | 37620 | 7: 45994439 | 37621 | 7: 45997784 | 37623 | 7: 46123501 | 37624 | 7: 46133078 |
| 37637 | 7: 46424169 | 37649 | 7: 46722010 | 37655 | 7: 46847399 | 37657 | 7: 46881748 | 37659 | 7: 46963800 |
| 37660 | 7: 46965141 | 37661 | 7: 46965986 | 37662 | 7: 46966917 | 37663 | 7: 46969143 | 37678 | 7: 47071431 |
| 37680 | 7: 47200029 | 37694 | 7: 47663425 | 37696 | 7: 47675422 | 37697 | 7: 47681261 | 37701 | 7: 47796975 |
| 37722 | 7: 48566329 | 37730 | 7: 48673881 | 37771 | 7: 50021166 | 37780 | 7: 50639584 | 37792 | 7: 51149038 |
| 37796 | 7: 51377867 | 37798 | 7: 51400935 | 37801 | 7: 51429431 | 37803 | 7: 51450812 | 37811 | 7: 51923554 |
| 37812 | 7: 52003650 | 37815 | 7: 52173201 | 37817 | 7: 52176528 | 37818 | 7: 52177126 | 37819 | 7: 52179498 |
| 37820 | 7: 52181770 | 37821 | 7: 52182473 | 37822 | 7: 52183049 | 37823 | 7: 52183519 | 37824 | 7: 52186624 |
| 37825 | 7: 52193152 | 37826 | 7: 52196823 | 37827 | 7: 52197432 | 37828 | 7: 52198757 | 37829 | 7: 52199982 |
| 37830 | 7: 52207035 | 37831 | 7: 52210371 | 37833 | 7: 52224828 | 37834 | 7: 52225521 | 37835 | 7: 52227549 |
| 37836 | 7: 52227592 | 37838 | 7: 52249785 | 37839 | 7: 52251276 | 37840 | 7: 52251297 | 37841 | 7: 52251426 |
| 37842 | 7: 52251546 | 37843 | 7: 52251618 | 37844 | 7: 52251861 | 37845 | 7: 52252987 | 37846 | 7: 52263699 |
| 37847 | 7: 52264079 | 37848 | 7: 52264430 | 37849 | 7: 52264511 | 37850 | 7: 52264761 | 37851 | 7: 52266394 |
| 37852 | 7: 52282234 | 37853 | 7: 52289509 | 37854 | 7: 52291347 | 37855 | 7: 52293091 | 37856 | 7: 52294120 |
| 37857 | 7: 52295099 | 37858 | 7: 52296408 | 37859 | 7: 52296468 | 37860 | 7: 52298384 | 37861 | 7: 52299162 |
| 37862 | 7: 52300813 | 37863 | 7: 52303764 | 37864 | 7: 52305125 | 37865 | 7: 52305218 | 37866 | 7: 52308210 |
| 37867 | 7: 52308534 | 37868 | 7: 52308687 | 37869 | 7: 52309821 | 37870 | 7: 52339995 | 37871 | 7: 52344653 |
| 37872 | 7: 52345469 | 37873 | 7: 52348265 | 37874 | 7: 52349069 | 37878 | 7: 52567577 | 37879 | 7: 52610395 |
| 37880 | 7: 52611688 | 37884 | 7: 52667909 | 37895 | 7: 52931391 | 37898 | 7: 53021130 | 3790 | 7: 53089284 |
| 37908 | 7: 53195304 | 37920 | 7: 53551894 | 37923 | 7: 53581842 | 37924 | 7: 53587134 | 37928 | 7: 53866382 |
| 37931 | 7: 53935965 | 37934 | 7: 53979466 | 37938 | 7: 54160608 | 37943 | 7: 54227894 | 37944 | 7: 54238880 |
| 37949 | 7: 54401837 | 37950 | 7: 54414044 | 37952 | 7: 54475662 | 37983 | 7: 55275367 | 37984 | 7: 55310804 |
| 37992 | 7: 55508633 | 37997 | 7: 55613829 | 38014 | 7: 56033788 | 38027 | 7: 56270767 | 38054 | 7: 57224558 |
| 38056 | 7: 57261914 | 38060 | 7: 57381076 | 38067 | 7: 57701637 | 38068 | 7: 57702653 | 38079 | 7: 57942576 |
| 38099 | 7: 58504274 | 38100 | 7: 58564407 | 38103 | 7: 58594636 | 38106 | 7: 58736129 | 38112 | 7: 58873109 |
| 38114 | 7: 58937938 | 38118 | 7: 59041978 | 38119 | 7: 59042119 | 38120 | 7: 59044072 | 38121 | 7: 59055433 |
| 38123 | 7: 59057188 | 38124 | 7: 59057293 | 38127 | 7: 59101278 | 38128 | 7: 59111622 | 38132 | 7: 59143825 |
| 38141 | 7: 59349638 | 38142 | 7: 59349648 | 38143 | 7: 59357865 | 38144 | 7: 59366049 | 38147 | 7: 59372999 |
| 38148 | 7: 59373109 | 38149 | 7: 59373145 | 38150 | 7: 59373890 | 38151 | 7: 59373911 | 38152 | 7: 59373987 |
| 38154 | 7: 59405369 | 38161 | 7: 59476011 | 38173 | 7: 59813378 | 38174 | 7: 59851233 | 38176 | 7: 59880943 |
| 38179 | 7: 59979404 | 38180 | 7: 59993171 | 38183 | 7: 60051047 | 38187 | 7: 60113080 | 38190 | 7: 60121204 |
| 38191 | 7: 60146715 | 38192 | 7: 60159491 | 38202 | 7: 60502888 | 38205 | 7: 60568272 | 38213 | 7: 60834283 |
| 38220 | 7: 60976830 | 38222 | 7: 60986666 | 38223 | 7: 60989492 | 38225 | 7: 61027791 | 38226 | 7: 61067590 |
| 38227 | 7: 61075693 | 38230 | 7: 61121888 | 38231 | 7: 61125177 | 38234 | 7: 61147314 | 38235 | 7: 61157706 |
| 38236 | 7: 61175816 | 38239 | 7: 61222597 | 38241 | 7: 61229844 | 38242 | 7: 61229883 | 38243 | 7: 61234323 |
| 38244 | 7: 61234418 | 38245 | 7: 61240972 | 38250 | 7: 61334946 | 38251 | 7: 61352203 | 38252 | 7: 61373465 |
| 38255 | 7: 61436635 | 38262 | 7: 61491892 | 38265 | 7: 61518179 | 38267 | 7: 61555353 | 38271 | 7: 61582159 |
| 38275 | 7: 61778550 | 38282 | 7: 62019992 | 38284 | 7: 62026620 | 38292 | 7: 62285057 | 38294 | 7: 62296401 |
| 38298 | 7: 62370721 | 38300 | 7: 62436131 | 38302 | 7: 62469301 | 38303 | 7: 62475081 | 38304 | 7: 62508971 |
| 38307 | 7: 62555449 | 38310 | 7: 62601203 | 38311 | 7: 62614056 | 38315 | 7: 62677859 | 38329 | 7: 62720115 |
| 38340 | 7: 62942190 | 38354 | 7: 63240676 | 38365 | 7: 63685100 | 38378 | 7: 63974136 | 38389 | 7: 64332584 |
| 38410 | 7: 64916386 | 38416 | 7: 65002376 | 38419 | 7: 65066376 | 38421 | 7: 65074340 | 38433 | 7: 65274641 |
| 38435 | 7: 65330563 | 38439 | 7: 65403716 | 38444 | 7: 65453830 | 38453 | 7: 65721041 | 38464 | 7: 65972504 |
| 38466 | 7: 65984655 | 38467 | 7: 66008306 | 38468 | 7: 66011532 | 38471 | 7: 66093673 | 38472 | 7: 66101631 |
| 38473 | 7: 66102398 | 38474 | 7: 66104176 | 38475 | 7: 66105023 | 38476 | 7: 66105843 | 38477 | 7: 66108875 |
| 38478 | 7: 66109426 | 38479 | 7: 66110363 | 38480 | 7: 66112663 | 38481 | 7: 66113133 | 38482 | 7: 66114775 |
| 38483 | 7: 66117158 | 38484 | 7: 66117914 | 38485 | 7: 66118467 | 38486 | 7: 66120071 | 38487 | 7: 66121243 |
| 38488 | 7: 66122354 | 38489 | 7: 66122876 | 38490 | 7: 66124410 | 38491 | 7: 66125761 | 38492 | 7: 66126649 |
| 38493 | 7: 66130675 | 38494 | 7: 66134907 | 38495 | 7: 66146626 | 38496 | 7: 66158927 | 38512 | 7: 66629354 |
| 38513 | 7: 66638097 | 38514 | 7: 66640996 | 38519 | 7: 66842880 | 38523 | 7: 66907257 | 38532 | 7: 67112605 |
| 38540 | 7: 67368109 | 38549 | 7: 67650949 | 38550 | 7: 67663732 | 38551 | 7: 67677225 | 38556 | 7: 67803883 |
| 38582 | 7: 68478076 | 38595 | 7: 68892260 | 38597 | 7: 68928960 | 38598 | 7: 68949630 | 38601 | 7: 68987039 |
| 38602 | 7: 68998753 | 38604 | 7: 69001013 | 38606 | 7: 69049520 | 38611 | 7: 69144296 | 38615 | 7: 69172516 |
| 38618 | 7: 69198573 | 38624 | 7: 69399622 | 38627 | 7: 69502402 | 38628 | 7: 69503500 | 38631 | 7: 69517359 |
| 38645 | 7: 69896069 | 38649 | 7: 69988788 | 38652 | 7: 70030654 | 38654 | 7: 70060807 | 38660 | 7: 70128127 |
| 38675 | 7: 70701637 | 38676 | 7: 70732259 | 38692 | 7: 71222059 | 38695 | 7: 71353347 | 38704 | 7: 71545001 |
| 38705 | 7: 71558907 | 38733 | 7: 72053452 | 38737 | 7: 72207894 | 38738 | 7: 72232062 | 38744 | 7: 72362246 |
| 38759 | 7: 72898450 | 38762 | 7: 72962658 | 38770 | 7: 73184647 | 38775 | 7: 73473535 | 38793 | 7: 73942821 |
| 38805 | 7: 74306314 | 38806 | 7: 74306327 | 38807 | 7: 74306379 | 38809 | 7: 74321645 | 38818 | 7: 74667948 |
| 38824 | 7: 74772424 | 38825 | 7: 74834470 | 38827 | 7: 74855482 | 38828 | 7: 74855556 | 38829 | 7: 74861008 |
| 38834 | 7: 74922579 | 38835 | 7: 74934509 | 38836 | 7: 74967965 | 38838 | 7: 75008858 | 38839 | 7: 75009945 |
| 38844 | 7: 75141363 | 38845 | 7: 75145201 | 38846 | 7: 75157547 | 38848 | 7: 75201764 | 38860 | 7: 75609551 |
| 38862 | 7: 75636089 | 38863 | 7: 75677642 | 38864 | 7: 75677689 | 38871 | 7: 75802119 | 38872 | 7: 75814579 |
| 38877 | 7: 75942645 | 38886 | 7: 76249772 | 38887 | 7: 76260400 | 38888 | 7: 76279595 | 38890 | 7: 76306268 |
| 38891 | 7: 76342697 | 38892 | 7: 76347497 | 38894 | 7: 76370749 | 38898 | 7: 76449574 | 38910 | 7: 76725621 |
| 38911 | 7: 76765051 | 38914 | 7: 76875833 | 38915 | 7: 76875983 | 38919 | 7: 77004376 | 38939 | 7: 77408753 |
| 38960 | 7: 77903085 | 38966 | 7: 77985310 | 38970 | 7: 78110195 | 38974 | 7: 78196572 | 38975 | 7: 78196650 |
| 38977 | 7: 78217247 | 39034 | 7: 79217925 | 39044 | 7: 79409160 | 39050 | 7: 79575037 | 39051 | 7: 79575100 |
| 39055 | 7: 79623429 | 39057 | 7: 79676699 | 39061 | 7: 79735833 | 39062 | 7: 79735898 | 39070 | 7: 79930582 |
| 39071 | 7: 79953397 | 39074 | 7: 80074984 | 39081 | 7: 80181414 | 39086 | 7: 80322587 | 39091 | 7: 80397326 |
| 39092 | 7: 80423038 | 39094 | 7: 80460624 | 39095 | 7: 80461082 | 39097 | 7: 80469480 | 39098 | 7: 80471094 |
| 39100 | 7: 80478643 | 39101 | 7: 80503605 | 39102 | 7: 80503765 | 39104 | 7: 80509969 | 39105 | 7: 80514758 |
| 39109 | 7: 80546745 | 39113 | 7: 80594054 | 39116 | 7: 80662788 | 39130 | 7: 81016584 | 39138 | 7: 81135163 |
| 39139 | 7: 81142008 | 39140 | 7: 81142055 | 39141 | 7: 81142172 | 39142 | 7: 81174306 | 39144 | 7: 81184476 |
| 39145 | 7: 81184486 | 39151 | 7: 81313732 | 39158 | 7: 81392597 | 39159 | 7: 81392623 | 39160 | 7: 81392634 |
| 39161 | 7: 81392650 | 39162 | 7: 81392653 | 39163 | 7: 81392665 | 39170 | 7: 81654928 | 39175 | 7: 81798512 |
| 39186 | 7: 82216490 | 39187 | 7: 82219959 | 39189 | 7: 82230892 | 39190 | 7: 82248295 | 39198 | 7: 82549435 |
| 39200 | 7: 82554305 | 39206 | 7: 82662900 | 39207 | 7: 82662928 | 39208 | 7: 82662929 | 39214 | 7: 82764185 |
| 39224 | 7: 83044183 | 39244 | 7: 83544032 | 39264 | 7: 84108168 | 39265 | 7: 84108203 | 39266 | 7: 84108212 |
| 39267 | 7: 84118928 | 39269 | 7: 84144485 | 39273 | 7: 84207697 | 39275 | 7: 84239228 | 39279 | 7: 84354399 |
| 39280 | 7: 84354448 | 39284 | 7: 84424155 | 39285 | 7: 84450073 | 39288 | 7: 84509794 | 39301 | 7: 85123948 |
| 39307 | 7: 85293142 | 39308 | 7: 85293161 | 39309 | 7: 85293169 | 39323 | 7: 85845513 | 39324 | 7: 85845564 |
| 39325 | 7: 85875827 | 39328 | 7: 85965693 | 39337 | 7: 86289890 | 39338 | 7: 86289929 | 39340 | 7: 86348377 |
| 39341 | 7: 86348400 | 39345 | 7: 86462459 | 39348 | 7: 86544147 | 39349 | 7: 86566118 | 39353 | 7: 86638215 |
| 39379 | 7: 87401188 | 39388 | 7: 87608805 | 39389 | 7: 87608884 | 39390 | 7: 87608885 | 39391 | 7: 87608911 |
| 39394 | 7: 87779029 | 39395 | 7: 87841745 | 39403 | 7: 88117633 | 39411 | 7: 88379769 | 39419 | 7: 88587478 |
| 39427 | 7: 88790932 | 39428 | 7: 88796649 | 39433 | 7: 88942654 | 39435 | 7: 88966573 | 39436 | 7: 89035670 |
| 39440 | 7: 89116248 | 39445 | 7: 89148646 | 39446 | 7: 89184830 | 39450 | 7: 89247931 | 39454 | 7: 89369998 |
| 39478 | 7: 90049487 | 39483 | 7: 90127121 | 39486 | 7: 90166602 | 39487 | 7: 90166627 | 39488 | 7: 90167634 |
| 39496 | 7: 90329807 | 39511 | 7: 90598533 | 39514 | 7: 90688919 | 39523 | 7: 90779825 | 39558 | 7: 90831595 |
| 39565 | 7: 90845831 | 39596 | 7: 91489178 | 39611 | 7: 91726773 | 39653 | 7: 92762601 | 39671 | 7: 93433611 |
| 39679 | 7: 93718483 | 39680 | 7: 93727310 | 39682 | 7: 93774135 | 39693 | 7: 94215835 | 39694 | 7: 94269949 |
| 39699 | 7: 94382446 | 39703 | 7: 94439368 | 39708 | 7: 94574747 | 39717 | 7: 94849950 | 39718 | 7: 94854494 |
| 39719 | 7: 94854604 | 39720 | 7: 94854627 | 39724 | 7: 94945483 | 39729 | 7: 95020011 | 39730 | 7: 95021828 |
| 39733 | 7: 95079030 | 39735 | 7: 95116563 | 39736 | 7: 95158884 | 39743 | 7: 95405216 | 39754 | 7: 95645495 |
| 39767 | 7: 95958530 | 39771 | 7: 96074523 | 39772 | 7: 96087310 | 39775 | 7: 96098877 | 39778 | 7: 96109770 |
| 39781 | 7: 96127017 | 39782 | 7: 96127023 | 39783 | 7: 96127094 | 39784 | 7: 96140233 | 39785 | 7: 96140250 |
| 39786 | 7: 96140274 | 39787 | 7: 96140286 | 39788 | 7: 96140316 | 39789 | 7: 96140325 | 39790 | 7: 96146797 |
| 39791 | 7: 96152289 | 39793 | 7: 96169102 | 39806 | 7: 96180272 | 39807 | 7: 96180277 | 39808 | 7: 96180291 |
| 39810 | 7: 96196608 | 39811 | 7: 96196642 | 39813 | 7: 96202522 | 39829 | 7: 96220995 | 39831 | 7: 96221193 |
| 39868 | 7: 96278344 | 39911 | 7: 96314599 | 39912 | 7: 96315078 | 39934 | 7: 96355415 | 39939 | 7: 96370734 |
| 39941 | 7: 96387738 | 39950 | 7: 96555097 | 39952 | 7: 96601732 | 39954 | 7: 96613640 | 39985 | 7: 97508892 |
| 39986 | 7: 97508905 | 39988 | 7: 97541930 | 39990 | 7: 97603739 | 39991 | 7: 97629287 | 39997 | 7: 97747540 |
| 40003 | 7: 97959533 | 40019 | 7: 98300666 | 40020 | 7: 98348623 | 40021 | 7: 98348636 | 40028 | 7: 98543164 |
| 40048 | 7: 99313338 | 40070 | 7: 99845218 | 40072 | 7: 99865657 | 40075 | 7: 99914902 | 40082 | 7: 100079502 |
| 40083 | 7: 100082753 | 40086 | 7: 100118655 | 40087 | 7: 100118835 | 40088 | 7: 100136909 | 40089 | 7: 100144865 |
| 40098 | 7: 100358134 | 40099 | 7: 100395807 | 40116 | 7: 100797612 | 40129 | 7: 101254706 | 40133 | 7: 101358810 |
| 40139 | 7: 101563788 | 40142 | 7: 101606446 | 40143 | 7: 101606448 | 40147 | 7: 101667972 | 40153 | 7: 101808436 |
| 40154 | 7: 101857168 | 40157 | 7: 102004868 | 40164 | 7: 102285994 | 40173 | 7: 102580748 | 40174 | 7: 102582243 |
| 40175 | 7: 102585831 | 40176 | 7: 102589674 | 40177 | 7: 102594188 | 40178 | 7: 102599028 | 40179 | 7: 102609048 |
| 40181 | 7: 102620944 | 40192 | 7: 102888318 | 40196 | 7: 102988342 | 40198 | 7: 103035483 | 40200 | 7: 103047606 |
| 40219 | 7: 103414818 | 40280 | 7: 105164324 | 40292 | 7: 105387524 | 40298 | 7: 105535552 | 40322 | 7: 106096645 |
| 40326 | 7: 106249472 | 40327 | 7: 106254713 | 40348 | 7: 106643847 | 40351 | 7: 106742504 | 40352 | 7: 106742506 |
| 40360 | 7: 106848432 | 40362 | 7: 106869396 | 40369 | 7: 107016860 | 40370 | 7: 107023813 | 40373 | 7: 107095006 |
| 40376 | 7: 107124835 | 40383 | 7: 107308750 | 40403 | 7: 108184476 | 40407 | 7: 108325718 | 40408 | 7: 108337537 |
| 40418 | 7: 108563367 | 40431 | 7: 108821941 | 40433 | 7: 108869029 | 40439 | 7: 108994618 | 40441 | 7: 109029038 |
| 40442 | 7: 109029077 | 40443 | 7: 109029102 | 40458 | 7: 109275280 | 40480 | 7: 109999314 | 40498 | 8: 373283 |
| 40506 | 8: 643950 | 40510 | 8: 764642 | 40511 | 8: 780102 | 40514 | 8: 800236 | 40515 | 8: 816422 |
| 40517 | 8: 844855 | 40520 | 8: 882426 | 40521 | 8: 883861 | 40535 | 8: 1082935 | 40536 | 8: 1086523 |
| 40548 | 8: 1362737 | 40552 | 8: 1579017 | 40564 | 8: 1757679 | 40571 | 8: 1832243 | 40579 | 8: 1939311 |
| 40594 | 8: 2317585 | 40606 | 8: 2582570 | 40616 | 8: 3008685 | 40620 | 8: 3178994 | 40621 | 8: 3219275 |
| 40630 | 8: 3400647 | 40631 | 8: 3416328 | 40632 | 8: 3416882 | 40667 | 8: 4251182 | 40668 | 8: 4273155 |
| 40669 | 8: 4274587 | 40670 | 8: 4276578 | 40671 | 8: 4280150 | 40672 | 8: 4283775 | 40685 | 8: 4639116 |
| 40687 | 8: 4644380 | 40688 | 8: 4644486 | 40690 | 8: 4708141 | 40693 | 8: 4759903 | 40698 | 8: 4908669 |
| 40707 | 8: 5249662 | 40708 | 8: 5270683 | 40709 | 8: 5270703 | 40710 | 8: 5270802 | 40711 | 8: 5270815 |
| 40712 | 8: 5332706 | 40728 | 8: 5760796 | 40735 | 8: 5869022 | 40741 | 8: 6027760 | 40765 | 8: 6756792 |
| 40766 | 8: 6764623 | 40785 | 8: 7181322 | 40786 | 8: 7181335 | 40803 | 8: 7581528 | 40805 | 8: 7596757 |
| 40806 | 8: 7598915 | 40807 | 8: 7601383 | 40808 | 8: 7603103 | 40809 | 8: 7611610 | 40810 | 8: 7618608 |
| 40811 | 8: 7619933 | 40812 | 8: 7620831 | 40813 | 8: 7625351 | 40818 | 8: 7818361 | 40826 | 8: 8001917 |
| 40832 | 8: 8175029 | 40842 | 8: 8393306 | 40844 | 8: 8477216 | 40848 | 8: 8618577 | 40857 | 8: 8769353 |
| 40866 | 8: 9042180 | 40868 | 8: 9063209 | 40869 | 8: 9119208 | 40889 | 8: 9619039 | 40890 | 8: 9619107 |
| 40891 | 8: 9619135 | 40892 | 8: 9623038 | 40893 | 8: 9627034 | 40894 | 8: 9627109 | 40895 | 8: 9627275 |
| 40896 | 8: 9627321 | 40905 | 8: 9890341 | 40906 | 8: 9901901 | 40910 | 8: 10031350 | 40917 | 8: 10339279 |
| 40925 | 8: 10604669 | 40931 | 8: 10696791 | 40934 | 8: 10712286 | 40935 | 8: 10712485 | 40936 | 8: 10712536 |
| 40939 | 8: 10783394 | 40942 | 8: 10854210 | 40943 | 8: 10854709 | 40952 | 8: 11049154 | 40955 | 8: 11128689 |
| 40980 | 8: 11615963 | 40981 | 8: 11626767 | 40983 | 8: 11655158 | 40985 | 8: 11693820 | 40990 | 8: 11853891 |
| 41001 | 8: 12089230 | 41008 | 8: 12344836 | 41012 | 8: 12433144 | 41026 | 8: 12914075 | 41036 | 8: 13348241 |
| 41038 | 8: 13393062 | 41047 | 8: 13722063 | 41097 | 8: 15148225 | 41103 | 8: 15333923 | 41109 | 8: 15473403 |
| 41113 | 8: 15497145 | 41114 | 8: 15497203 | 41115 | 8: 15497248 | 41116 | 8: 15533442 | 41120 | 8: 15590887 |
| 41121 | 8: 15590953 | 41124 | 8: 15685122 | 41128 | 8: 15765706 | 41133 | 8: 15967637 | 41143 | 8: 16338822 |
| 41145 | 8: 16399613 | 41155 | 8: 16592732 | 41158 | 8: 16699848 | 41162 | 8: 16791579 | 41163 | 8: 16792851 |
| 41171 | 8: 16926726 | 41174 | 8: 16945156 | 41178 | 8: 17032464 | 41179 | 8: 17032475 | 41180 | 8: 17032488 |
| 41182 | 8: 17048252 | 41183 | 8: 17055738 | 41184 | 8: 17055749 | 41185 | 8: 17055775 | 41186 | 8: 17056869 |
| 41187 | 8: 17061563 | 41188 | 8: 17064508 | 41193 | 8: 17304347 | 41195 | 8: 17305220 | 41196 | 8: 17307048 |
| 41197 | 8: 17307131 | 41206 | 8: 17548023 | 41231 | 8: 18280322 | 41242 | 8: 18613707 | 41246 | 8: 18768280 |
| 41257 | 8: 19052433 | 41288 | 8: 19935582 | 41301 | 8: 20342622 | 41342 | 8: 21329932 | 41361 | 8: 21984030 |
| 41363 | 8: 22082837 | 41364 | 8: 22100760 | 41365 | 8: 22113575 | 41396 | 8: 23324599 | 41401 | 8: 23427337 |
| 41421 | 8: 24057660 | 41434 | 8: 24296988 | 41440 | 8: 24394806 | 41442 | 8: 24405999 | 41444 | 8: 24443570 |
| 41450 | 8: 24643364 | 41452 | 8: 24674588 | 41454 | 8: 24687934 | 41455 | 8: 24689851 | 41456 | 8: 24692069 |
| 41457 | 8: 24700212 | 41459 | 8: 24720840 | 41462 | 8: 24752172 | 41466 | 8: 24771365 | 41467 | 8: 24786333 |
| 41468 | 8: 24793655 | 41469 | 8: 24802374 | 41470 | 8: 24812615 | 41471 | 8: 24820473 | 41472 | 8: 24827262 |
| 41475 | 8: 24874038 | 41476 | 8: 24875382 | 41477 | 8: 24877159 | 41478 | 8: 24877700 | 41479 | 8: 24878235 |
| 41480 | 8: 24878922 | 41481 | 8: 24888678 | 41482 | 8: 24891103 | 41483 | 8: 24895594 | 41485 | 8: 24933323 |
| 41486 | 8: 24938771 | 41487 | 8: 24940757 | 41488 | 8: 24955657 | 41489 | 8: 24957890 | 41490 | 8: 24961237 |
| 41491 | 8: 24966416 | 41492 | 8: 24974478 | 41493 | 8: 24979200 | 41494 | 8: 24988001 | 41495 | 8: 24992186 |
| 41496 | 8: 24996358 | 41497 | 8: 25006948 | 41498 | 8: 25013387 | 41499 | 8: 25035664 | 41500 | 8: 25041072 |
| 41501 | 8: 25059424 | 41506 | 8: 25124953 | 41507 | 8: 25126729 | 41509 | 8: 25140406 | 41510 | 8: 25144505 |
| 41511 | 8: 25146183 | 41512 | 8: 25148717 | 41513 | 8: 25150222 | 41514 | 8: 25152059 | 41515 | 8: 25154593 |
| 41516 | 8: 25157388 | 41517 | 8: 25160394 | 41518 | 8: 25165311 | 41519 | 8: 25166943 | 41520 | 8: 25169477 |
| 41522 | 8: 25174754 | 41523 | 8: 25177709 | 41524 | 8: 25180230 | 41525 | 8: 25185692 | 41526 | 8: 25188439 |
| 41527 | 8: 25198273 | 41528 | 8: 25200755 | 41530 | 8: 25214312 | 41531 | 8: 25227176 | 41532 | 8: 25233627 |
| 41533 | 8: 25235124 | 41534 | 8: 25238388 | 41535 | 8: 25240252 | 41537 | 8: 25243485 | 41538 | 8: 25254140 |
| 41539 | 8: 25257480 | 41540 | 8: 25259038 | 41541 | 8: 25261263 | 41542 | 8: 25263852 | 41543 | 8: 25265485 |
| 41544 | 8: 25266937 | 41545 | 8: 25268844 | 41546 | 8: 25274693 | 41547 | 8: 25282832 | 41548 | 8: 25294627 |
| 41557 | 8: 25487377 | 41558 | 8: 25500162 | 41561 | 8: 25551702 | 41580 | 8: 25993221 | 41584 | 8: 26067879 |
| 41602 | 8: 26560396 | 41604 | 8: 26591338 | 41614 | 8: 26825483 | 41621 | 8: 26909158 | 41631 | 8: 27313786 |
| 41633 | 8: 27383296 | 41637 | 8: 27558131 | 41649 | 8: 27894769 | 41652 | 8: 27969801 | 41654 | 8: 28067472 |
| 41664 | 8: 28411601 | 41665 | 8: 28411658 | 41670 | 8: 28576597 | 41673 | 8: 28652470 | 41678 | 8: 28903569 |
| 41679 | 8: 28935823 | 41691 | 8: 29203761 | 41702 | 8: 29452933 | 41706 | 8: 29509148 | 41707 | 8: 29543012 |
| 41728 | 8: 29972472 | 41730 | 8: 30031385 | 41734 | 8: 30076221 | 41737 | 8: 30253112 | 41750 | 8: 30514176 |
| 41754 | 8: 30666192 | 41757 | 8: 30707536 | 41764 | 8: 30964101 | 41765 | 8: 30965820 | 41767 | 8: 31027893 |
| 41769 | 8: 31049984 | 41770 | 8: 31056192 | 41773 | 8: 31128535 | 41776 | 8: 31166613 | 41780 | 8: 31290392 |
| 41781 | 8: 31300884 | 41785 | 8: 31442029 | 41786 | 8: 31453763 | 41790 | 8: 31579785 | 41793 | 8: 31643340 |
| 41801 | 8: 31867242 | 41802 | 8: 31895156 | 41859 | 8: 33917004 | 41888 | 8: 34615024 | 41894 | 8: 34800931 |
| 41895 | 8: 34800977 | 41896 | 8: 34801009 | 41919 | 8: 35445047 | 41923 | 8: 35459817 | 41936 | 8: 35833781 |
| 41938 | 8: 35859948 | 41943 | 8: 35930089 | 41953 | 8: 36065706 | 41956 | 8: 36096463 | 41958 | 8: 36187712 |
| 41962 | 8: 36233237 | 41965 | 8: 36292654 | 41968 | 8: 36343251 | 41985 | 8: 36842382 | 41986 | 8: 36851180 |
| 41995 | 8: 37031806 | 41996 | 8: 37054784 | 41998 | 8: 37095387 | 42008 | 8: 37407050 | 42010 | 8: 37435996 |
| 42023 | 8: 37797692 | 42024 | 8: 37854305 | 42028 | 8: 37928176 | 42044 | 8: 38675399 | 42047 | 8: 38691615 |
| 42056 | 8: 39019428 | 42061 | 8: 39290623 | 42067 | 8: 39583278 | 42068 | 8: 39584890 | 42069 | 8: 39621719 |
| 42074 | 8: 39696110 | 42075 | 8: 39718793 | 42077 | 8: 39768968 | 42078 | 8: 39769005 | 42079 | 8: 39809755 |
| 42081 | 8: 39864901 | 42091 | 8: 40003850 | 42101 | 8: 40171823 | 42102 | 8: 40219628 | 42103 | 8: 40219668 |
| 42105 | 8: 40257959 | 42106 | 8: 40287413 | 42107 | 8: 40306339 | 42109 | 8: 40334191 | 42110 | 8: 40334321 |
| 42114 | 8: 40410096 | 42131 | 8: 40743770 | 42135 | 8: 40830870 | 42150 | 8: 41247220 | 42152 | 8: 41309293 |
| 42165 | 8: 41611046 | 42168 | 8: 41682500 | 42169 | 8: 41693466 | 42170 | 8: 41697104 | 42174 | 8: 41759986 |
| 42176 | 8: 41790111 | 42179 | 8: 41858426 | 42180 | 8: 41890619 | 42181 | 8: 41897019 | 42199 | 8: 42436416 |
| 42211 | 8: 42709975 | 42212 | 8: 42720374 | 42216 | 8: 42880518 | 42220 | 8: 42958668 | 42221 | 8: 42958711 |
| 42222 | 8: 42958836 | 42226 | 8: 43016661 | 42236 | 8: 43367984 | 42245 | 8: 43812387 | 42246 | 8: 43815004 |
| 42250 | 8: 43957564 | 42255 | 8: 43998136 | 42256 | 8: 43998213 | 42258 | 8: 44010856 | 42269 | 8: 44431429 |
| 42273 | 8: 44588721 | 42282 | 8: 44879292 | 42283 | 8: 44895474 | 42285 | 8: 44916012 | 42295 | 8: 45238924 |
| 42301 | 8: 45399787 | 42304 | 8: 45427010 | 42306 | 8: 45447403 | 42309 | 8: 45519975 | 42310 | 8: 45544435 |
| 42314 | 8: 45672590 | 42316 | 8: 45691728 | 42321 | 8: 45748288 | 42327 | 8: 45881905 | 42328 | 8: 45949542 |
| 42333 | 8: 46053215 | 42334 | 8: 46058853 | 42347 | 8: 46184772 | 42352 | 8: 46322367 | 42358 | 8: 46514754 |
| 42362 | 8: 46662471 | 42363 | 8: 46688442 | 42367 | 8: 46794717 | 42378 | 8: 47069272 | 42379 | 8: 47096555 |
| 42383 | 8: 47242828 | 42384 | 8: 47301441 | 42386 | 8: 47301545 | 42396 | 8: 47442795 | 42397 | 8: 47454300 |
| 42398 | 8: 47468132 | 42413 | 8: 47827950 | 42416 | 8: 47959914 | 42443 | 8: 48870474 | 42452 | 8: 48980291 |
| 42455 | 8: 49060228 | 42456 | 8: 49060267 | 42457 | 8: 49060352 | 42458 | 8: 49071203 | 42459 | 8: 49082742 |
| 42465 | 8: 49227434 | 42466 | 8: 49248066 | 42470 | 8: 49305124 | 42471 | 8: 49328971 | 42472 | 8: 49332342 |
| 42484 | 8: 49820121 | 42490 | 8: 50120835 | 42493 | 8: 50158365 | 42496 | 8: 50210739 | 42502 | 8: 50343730 |
| 42505 | 8: 50410404 | 42526 | 8: 51083850 | 42529 | 8: 51149379 | 42530 | 8: 51154605 | 42537 | 8: 51409152 |
| 42549 | 8: 51645019 | 42553 | 8: 51697635 | 42563 | 8: 51983681 | 42569 | 8: 52152405 | 42570 | 8: 52157895 |
| 42573 | 8: 52201603 | 42584 | 8: 52496347 | 42593 | 8: 52683991 | 42597 | 8: 52775082 | 42606 | 8: 53099576 |
| 42607 | 8: 53099649 | 42608 | 8: 53099684 | 42618 | 8: 53465873 | 42622 | 8: 53559314 | 42626 | 8: 53663149 |
| 42628 | 8: 53760217 | 42643 | 8: 54223148 | 42650 | 8: 54393899 | 42663 | 8: 54661982 | 42665 | 8: 54767805 |
| 42666 | 8: 54767855 | 42676 | 8: 54981068 | 42677 | 8: 54981074 | 42678 | 8: 54981156 | 42693 | 8: 55456387 |
| 42697 | 8: 55552471 | 42699 | 8: 55552575 | 42707 | 8: 55861711 | 42708 | 8: 55861771 | 42709 | 8: 55869592 |
| 42711 | 8: 55889240 | 42718 | 8: 56082094 | 42719 | 8: 56082109 | 42723 | 8: 56355997 | 42732 | 8: 56476308 |
| 42736 | 8: 56567532 | 42749 | 8: 56898409 | 42752 | 8: 56940553 | 42755 | 8: 57067743 | 42756 | 8: 57083802 |
| 42757 | 8: 57097646 | 42769 | 8: 57431848 | 42770 | 8: 57436089 | 42771 | 8: 57441513 | 42774 | 8: 57491864 |
| 42777 | 8: 57503925 | 42781 | 8: 57561608 | 42784 | 8: 57609698 | 42785 | 8: 57624471 | 42794 | 8: 58010694 |
| 42796 | 8: 58065599 | 42799 | 8: 58159003 | 42803 | 8: 58281685 | 42817 | 8: 58837870 | 42827 | 8: 59185961 |
| 42831 | 8: 59304332 | 42833 | 8: 59343826 | 42843 | 8: 59578341 | 42845 | 8: 59585372 | 42846 | 8: 59591203 |
| 42847 | 8: 59595742 | 42853 | 8: 59747078 | 42856 | 8: 59839831 | 42861 | 8: 59908902 | 42867 | 8: 59979908 |
| 42869 | 8: 59997834 | 42873 | 8: 60218203 | 42874 | 8: 60232849 | 42875 | 8: 60252660 | 42877 | 8: 60376601 |
| 42878 | 8: 60387912 | 42880 | 8: 60393687 | 42881 | 8: 60407147 | 42882 | 8: 60408080 | 42883 | 8: 60413868 |
| 42885 | 8: 60422237 | 42887 | 8: 60434309 | 42888 | 8: 60445473 | 42893 | 8: 60483449 | 42901 | 8: 60773154 |
| 42906 | 8: 60847978 | 42928 | 8: 61569513 | 42931 | 8: 61676872 | 42953 | 8: 62298007 | 42955 | 8: 62361352 |
| 42956 | 8: 62370401 | 42957 | 8: 62370423 | 42960 | 8: 62476415 | 42961 | 8: 62476489 | 42962 | 8: 62476593 |
| 42963 | 8: 62481551 | 42964 | 8: 62481563 | 42965 | 8: 62492081 | 42966 | 8: 62492171 | 42967 | 8: 62492207 |
| 42969 | 8: 62554727 | 42970 | 8: 62556949 | 42977 | 8: 62779236 | 42980 | 8: 62823371 | 42985 | 8: 62978731 |
| 42986 | 8: 62978820 | 42989 | 8: 63076318 | 42991 | 8: 63182958 | 42998 | 8: 63428149 | 43008 | 8: 63577043 |
| 43025 | 8: 64001632 | 43026 | 8: 64003121 | 43031 | 8: 64111218 | 43038 | 8: 64443505 | 43041 | 8: 64480556 |
| 43042 | 8: 64512625 | 43050 | 8: 64890322 | 43059 | 8: 65128134 | 43060 | 8: 65147458 | 43063 | 8: 65217066 |
| 43064 | 8: 65219496 | 43065 | 8: 65219567 | 43073 | 8: 65486798 | 43074 | 8: 65486879 | 43075 | 8: 65486964 |
| 43076 | 8: 65487030 | 43077 | 8: 65487045 | 43078 | 8: 65487167 | 43079 | 8: 65487520 | 43080 | 8: 65506614 |
| 43092 | 8: 66005380 | 43094 | 8: 66041697 | 43095 | 8: 66071027 | 43096 | 8: 66071753 | 43099 | 8: 66095067 |
| 43100 | 8: 66095107 | 43102 | 8: 66110177 | 43103 | 8: 66110963 | 43104 | 8: 66111005 | 43117 | 8: 66467235 |
| 43129 | 8: 66780866 | 43130 | 8: 66781633 | 43132 | 8: 66784915 | 43134 | 8: 66833433 | 43135 | 8: 66833492 |
| 43139 | 8: 66987197 | 43140 | 8: 66999877 | 43141 | 8: 67000007 | 43143 | 8: 67013017 | 43146 | 8: 67072138 |
| 43148 | 8: 67105385 | 43149 | 8: 67105408 | 43150 | 8: 67121705 | 43153 | 8: 67145134 | 43154 | 8: 67154528 |
| 43158 | 8: 67392786 | 43162 | 8: 67566051 | 43164 | 8: 67576272 | 43166 | 8: 67607555 | 43167 | 8: 67613943 |
| 43170 | 8: 67704512 | 43171 | 8: 67731230 | 43182 | 8: 67844754 | 43183 | 8: 67844773 | 43200 | 8: 68399674 |
| 43216 | 8: 68844096 | 43217 | 8: 68866548 | 43218 | 8: 68866614 | 43223 | 8: 68960840 | 43224 | 8: 69009944 |
| 43227 | 8: 69053764 | 43236 | 8: 69156564 | 43237 | 8: 69156580 | 43239 | 8: 69252951 | 43241 | 8: 69325956 |
| 43242 | 8: 69421607 | 43258 | 8: 69882196 | 43261 | 8: 69947806 | 43262 | 8: 69974129 | 43263 | 8: 70014064 |
| 43264 | 8: 70029487 | 43265 | 8: 70039617 | 43280 | 8: 70405010 | 43286 | 8: 70466364 | 43288 | 8: 70482064 |
| 43289 | 8: 70489717 | 43290 | 8: 70492869 | 43292 | 8: 70501163 | 43297 | 8: 70592634 | 43303 | 8: 70815570 |
| 43306 | 8: 70893052 | 43310 | 8: 70958608 | 43319 | 8: 71238004 | 43336 | 8: 71880675 | 43360 | 8: 72468833 |
| 43361 | 8: 72468916 | 43362 | 8: 72515403 | 43364 | 8: 72551579 | 43366 | 8: 72558376 | 43367 | 8: 72561704 |
| 43372 | 8: 72825901 | 43373 | 8: 72828377 | 43375 | 8: 72837275 | 43376 | 8: 72844608 | 43377 | 8: 72845970 |
| 43378 | 8: 72904078 | 43380 | 8: 72930313 | 43404 | 8: 73494061 | 43406 | 8: 73515301 | 43412 | 8: 73656442 |
| 43423 | 8: 73915262 | 43432 | 8: 74168959 | 43436 | 8: 74325878 | 43440 | 8: 74465417 | 43444 | 8: 74553402 |
| 43445 | 8: 74558074 | 43446 | 8: 74558704 | 43452 | 8: 74712678 | 43458 | 8: 74817911 | 43462 | 8: 74863602 |
| 43463 | 8: 74883099 | 43467 | 8: 75123238 | 43471 | 8: 75268745 | 43477 | 8: 75477646 | 43481 | 8: 75562796 |
| 43484 | 8: 75700129 | 43487 | 8: 75740079 | 43490 | 8: 75853292 | 43497 | 8: 76175098 | 43507 | 8: 76433813 |
| 43508 | 8: 76434785 | 43509 | 8: 76434815 | 43511 | 8: 76464537 | 43513 | 8: 76492709 | 43515 | 8: 76515741 |
| 43521 | 8: 76664342 | 43522 | 8: 76681835 | 43523 | 8: 76684305 | 43524 | 8: 76687450 | 43525 | 8: 76703859 |
| 43526 | 8: 76709352 | 43529 | 8: 76742010 | 43530 | 8: 76752971 | 43531 | 8: 76754631 | 43532 | 8: 76760562 |
| 43533 | 8: 76762856 | 43534 | 8: 76764812 | 43535 | 8: 76768935 | 43538 | 8: 76828964 | 43539 | 8: 76835585 |
| 43540 | 8: 76839168 | 43541 | 8: 76841937 | 43542 | 8: 76843765 | 43543 | 8: 76844513 | 43544 | 8: 76846849 |
| 43545 | 8: 76850281 | 43546 | 8: 76854581 | 43547 | 8: 76870958 | 43548 | 8: 76873029 | 43551 | 8: 76975704 |
| 43553 | 8: 76993039 | 43554 | 8: 77006329 | 43556 | 8: 77136545 | 43564 | 8: 77562434 | 43581 | 8: 78166070 |
| 43589 | 8: 78358551 | 43595 | 8: 78556780 | 43616 | 8: 79447931 | 43642 | 8: 80268872 | 43648 | 8: 80405770 |
| 43652 | 8: 80565130 | 43656 | 8: 80613713 | 43660 | 8: 80836273 | 43669 | 8: 80974113 | 43686 | 8: 81278385 |
| 43692 | 8: 81389738 | 43695 | 8: 81454715 | 43696 | 8: 81454750 | 43698 | 8: 81487284 | 43699 | 8: 81487295 |
| 43704 | 8: 81616321 | 43712 | 8: 81744964 | 43716 | 8: 81860516 | 43727 | 8: 82113238 | 43728 | 8: 82116405 |
| 43730 | 8: 82160329 | 43751 | 8: 82676827 | 43752 | 8: 82677581 | 43753 | 8: 82677988 | 43759 | 8: 82823172 |
| 43762 | 8: 82865703 | 43769 | 8: 83085781 | 43782 | 8: 83515046 | 43790 | 8: 83738651 | 43794 | 8: 83793151 |
| 43795 | 8: 83793154 | 43796 | 8: 83794591 | 43797 | 8: 83794760 | 43799 | 8: 83842360 | 43803 | 8: 83879316 |
| 43808 | 8: 83976437 | 43809 | 8: 84045184 | 43811 | 8: 84100704 | 43812 | 8: 84123211 | 43813 | 8: 84134245 |
| 43815 | 8: 84172945 | 43829 | 8: 84494416 | 43835 | 8: 84811346 | 43842 | 8: 84912804 | 43858 | 8: 85366261 |
| 43873 | 8: 85725330 | 43875 | 8: 85804392 | 43884 | 8: 85987078 | 43888 | 8: 86079018 | 43891 | 8: 86115895 |
| 43892 | 8: 86133024 | 43895 | 8: 86219410 | 43896 | 8: 86226370 | 43905 | 8: 86537816 | 43912 | 8: 86719772 |
| 43914 | 8: 86756176 | 43916 | 8: 86779557 | 43917 | 8: 86793653 | 43920 | 8: 86935675 | 43921 | 8: 86961975 |
| 43949 | 8: 87523043 | 43955 | 8: 87701857 | 43967 | 8: 88033337 | 43968 | 8: 88126576 | 43977 | 8: 88363375 |
| 43986 | 8: 88562919 | 43987 | 8: 88572133 | 43995 | 8: 88712829 | 43997 | 8: 88762685 | 44001 | 8: 88829394 |
| 44007 | 8: 88990195 | 44013 | 8: 89098414 | 44014 | 8: 89113184 | 44016 | 8: 89140453 | 44018 | 8: 89141946 |
| 44019 | 8: 89141971 | 44020 | 8: 89142000 | 44021 | 8: 89142063 | 44024 | 8: 89269473 | 44033 | 8: 89533107 |
| 44034 | 8: 89533116 | 44035 | 8: 89533121 | 44037 | 8: 89557638 | 44041 | 8: 89571402 | 44046 | 8: 89722190 |
| 44049 | 8: 89770693 | 44056 | 8: 89926318 | 44059 | 8: 90070089 | 44062 | 8: 90193893 | 44084 | 8: 90853473 |
| 44085 | 8: 90853557 | 44086 | 8: 90853588 | 44087 | 8: 90853599 | 44101 | 8: 91152034 | 44102 | 8: 91161810 |
| 44106 | 8: 91289222 | 44110 | 8: 91381933 | 44111 | 8: 91382574 | 44129 | 8: 91948504 | 44137 | 8: 91989858 |
| 44138 | 8: 91995185 | 44143 | 8: 92008855 | 44149 | 8: 92136613 | 44168 | 8: 92747513 | 44178 | 8: 93068614 |
| 44179 | 8: 93068725 | 44204 | 8: 93841029 | 44208 | 8: 94082045 | 44212 | 8: 94260204 | 44220 | 8: 94620469 |
| 44221 | 8: 94620534 | 44222 | 8: 94621015 | 44223 | 8: 94623988 | 44224 | 8: 94624900 | 44225 | 8: 94672379 |
| 44226 | 8: 94690978 | 44232 | 8: 94939320 | 44241 | 8: 95337173 | 44259 | 8: 95796796 | 44263 | 8: 95951526 |
| 44264 | 8: 95977921 | 44266 | 8: 96012156 | 44272 | 8: 96165982 | 44273 | 8: 96193360 | 44281 | 8: 96384779 |
| 44282 | 8: 96445578 | 44289 | 8: 96573262 | 44308 | 8: 97157793 | 44317 | 8: 97299078 | 44319 | 8: 97368811 |
| 44326 | 8: 97495202 | 44327 | 8: 97557161 | 44329 | 8: 97577094 | 44330 | 8: 97601680 | 44335 | 8: 97795768 |
| 44350 | 8: 98245729 | 44360 | 8: 98592583 | 44362 | 8: 98625126 | 44367 | 8: 98741152 | 44368 | 8: 98785042 |
| 44370 | 8: 98829420 | 44375 | 8: 98919680 | 44386 | 8: 99142994 | 44392 | 8: 99207386 | 44397 | 8: 99291886 |
| 44400 | 8: 99333533 | 44404 | 8: 99388416 | 44405 | 8: 99406177 | 44407 | 8: 99412670 | 44409 | 8: 99476683 |
| 44413 | 8: 99525142 | 44415 | 8: 99551509 | 44425 | 8: 99654305 | 44429 | 8: 99703737 | 44435 | 8: 99959755 |
| 44436 | 8: 99982505 | 44437 | 8: 100010762 | 44446 | 8: 100374163 | 44448 | 8: 100401670 | 44449 | 8: 100409615 |
| 44453 | 8: 100428898 | 44455 | 8: 100470224 | 44466 | 8: 100654637 | 44471 | 8: 100832272 | 44472 | 8: 100876059 |
| 44478 | 8: 101026237 | 44482 | 8: 101247151 | 44485 | 8: 101301768 | 44486 | 8: 101312150 | 44494 | 8: 101441924 |
| 44496 | 8: 101485459 | 44500 | 8: 101643451 | 44502 | 8: 101701776 | 44506 | 8: 101779830 | 44507 | 8: 101800844 |
| 44517 | 8: 102113941 | 44523 | 8: 102234774 | 44525 | 8: 102290809 | 44526 | 8: 102291109 | 44527 | 8: 102291157 |
| 44528 | 8: 102291228 | 44529 | 8: 102291230 | 44530 | 8: 102291256 | 44531 | 8: 102291263 | 44532 | 8: 102293933 |
| 44533 | 8: 102293974 | 44534 | 8: 102299598 | 44535 | 8: 102299601 | 44536 | 8: 102305536 | 44538 | 8: 102377736 |
| 44540 | 8: 102402762 | 44541 | 8: 102408863 | 44542 | 8: 102411405 | 44546 | 8: 102434252 | 44554 | 8: 102594527 |
| 44555 | 8: 102594773 | 44556 | 8: 102595590 | 44557 | 8: 102617174 | 44558 | 8: 102618622 | 44560 | 8: 102626997 |
| 44561 | 8: 102634985 | 44562 | 8: 102640018 | 44564 | 8: 102642339 | 44566 | 8: 102645680 | 44567 | 8: 102648250 |
| 44568 | 8: 102648741 | 44577 | 8: 102834472 | 44579 | 8: 102889304 | 44583 | 8: 102931846 | 44596 | 8: 103307658 |
| 44597 | 8: 103316620 | 44598 | 8: 103323065 | 44599 | 8: 103323527 | 44600 | 8: 103415237 | 44601 | 8: 103442278 |
| 44602 | 8: 103449094 | 44604 | 8: 103484976 | 44605 | 8: 103489848 | 44606 | 8: 103500362 | 44607 | 8: 103516850 |
| 44608 | 8: 103529815 | 44609 | 8: 103560463 | 44610 | 8: 103572704 | 44611 | 8: 103575282 | 44612 | 8: 103580891 |
| 44613 | 8: 103584228 | 44614 | 8: 103588272 | 44615 | 8: 103589059 | 44616 | 8: 103591535 | 44618 | 8: 103596979 |
| 44619 | 8: 103598675 | 44620 | 8: 103603170 | 44621 | 8: 103612096 | 44622 | 8: 103612794 | 44624 | 8: 103623951 |
| 44625 | 8: 103634947 | 44626 | 8: 103640204 | 44627 | 8: 103645354 | 44628 | 8: 103649615 | 44630 | 8: 103673828 |
| 44634 | 8: 103783688 | 44646 | 8: 104113096 | 44647 | 8: 104114568 | 44650 | 8: 104204807 | 44654 | 8: 104251576 |
| 44655 | 8: 104271837 | 44658 | 8: 104292759 | 44659 | 8: 104322730 | 44661 | 8: 104414493 | 44663 | 8: 104420746 |
| 44669 | 8: 104546178 | 44674 | 8: 104630353 | 44679 | 8: 104760159 | 44681 | 8: 104815425 | 44694 | 8: 105160429 |
| 44716 | 8: 105525844 | 44719 | 8: 105592649 | 44722 | 8: 105630176 | 44725 | 8: 105696501 | 44727 | 8: 105765759 |
| 44728 | 8: 105774110 | 44732 | 8: 105846896 | 44739 | 8: 106082143 | 44744 | 8: 106427938 | 44759 | 8: 106936156 |
| 44760 | 8: 106961448 | 44761 | 8: 106981761 | 44762 | 8: 107001526 | 44808 | 8: 108127783 | 44829 | 8: 108978247 |
| 44850 | 8: 109853668 | 44855 | 8: 110002325 | 44857 | 8: 110059433 | 44860 | 8: 110157715 | 44862 | 8: 110244165 |
| 44863 | 8: 110244337 | 44865 | 8: 110248495 | 44866 | 8: 110251397 | 44870 | 8: 110445148 | 44871 | 8: 110445202 |
| 44872 | 8: 110445222 | 44874 | 8: 110540868 | 44880 | 8: 110749028 | 44882 | 8: 110764849 | 44888 | 8: 110972844 |
| 44890 | 8: 110979223 | 44892 | 8: 111002543 | 44893 | 8: 111081452 | 44897 | 8: 111284112 | 44932 | 9: 349617 |
| 44935 | 9: 449265 | 44965 | 9: 761293 | 44966 | 9: 761398 | 44970 | 9: 946489 | 44983 | 9: 1228475 |
| 44984 | 9: 1228520 | 44985 | 9: 1228587 | 44986 | 9: 1228611 | 44987 | 9: 1228669 | 44997 | 9: 1530507 |
| 44998 | 9: 1530528 | 45013 | 9: 1926540 | 45016 | 9: 2022731 | 45017 | 9: 2022740 | 45084 | 9: 4060756 |
| 45090 | 9: 4309847 | 45101 | 9: 4645222 | 45115 | 9: 5086559 | 45159 | 9: 6390476 | 45161 | 9: 6450647 |
| 45163 | 9: 6559884 | 45174 | 9: 6887577 | 45189 | 9: 7542350 | 45206 | 9: 7865211 | 45219 | 9: 8218235 |
| 45220 | 9: 8218462 | 45223 | 9: 8257979 | 45224 | 9: 8258011 | 45225 | 9: 8258047 | 45241 | 9: 8662565 |
| 45242 | 9: 8691938 | 45245 | 9: 8740852 | 45246 | 9: 8740922 | 45247 | 9: 8741004 | 45255 | 9: 9002672 |
| 45259 | 9: 9073133 | 45265 | 9: 9212931 | 45269 | 9: 9363423 | 45281 | 9: 9866377 | 45288 | 9: 10026119 |
| 45290 | 9: 10179658 | 45293 | 9: 10229582 | 45295 | 9: 10270107 | 45302 | 9: 10376631 | 45307 | 9: 10412914 |
| 45343 | 9: 10650283 | 45377 | 9: 10813074 | 45393 | 9: 11379384 | 45398 | 9: 11488885 | 45413 | 9: 11816817 |
| 45418 | 9: 11933227 | 45428 | 9: 12091110 | 45431 | 9: 12153363 | 45433 | 9: 12204845 | 45435 | 9: 12250871 |
| 45445 | 9: 12499635 | 45446 | 9: 12509673 | 45452 | 9: 12645170 | 45471 | 9: 12766564 | 45481 | 9: 13100266 |
| 45485 | 9: 13105495 | 45491 | 9: 13132484 | 45496 | 9: 13287682 | 45498 | 9: 13361022 | 45503 | 9: 13494908 |
| 45509 | 9: 13684115 | 45510 | 9: 13698663 | 45517 | 9: 13880641 | 45520 | 9: 13989518 | 45523 | 9: 14095633 |
| 45524 | 9: 14124920 | 45527 | 9: 14203195 | 45540 | 9: 14468437 | 45545 | 9: 14564006 | 45548 | 9: 14665440 |
| 45550 | 9: 14717693 | 45564 | 9: 15013572 | 45568 | 9: 15038319 | 45570 | 9: 15071350 | 45571 | 9: 15081586 |
| 45574 | 9: 15123580 | 45577 | 9: 15163893 | 45578 | 9: 15175648 | 45585 | 9: 15369931 | 45587 | 9: 15406354 |
| 45595 | 9: 15497571 | 45596 | 9: 15499363 | 45597 | 9: 15499412 | 45598 | 9: 15499535 | 45599 | 9: 15511160 |
| 45602 | 9: 15527205 | 45604 | 9: 15548110 | 45605 | 9: 15553535 | 45606 | 9: 15558085 | 45607 | 9: 15560500 |
| 45608 | 9: 15560527 | 45609 | 9: 15562142 | 45623 | 9: 15687216 | 45631 | 9: 15868930 | 45644 | 9: 16138159 |
| 45653 | 9: 16384844 | 45657 | 9: 16537269 | 45674 | 9: 17031734 | 45675 | 9: 17031771 | 45687 | 9: 17461271 |
| 45692 | 9: 17595872 | 45717 | 9: 18430308 | 45721 | 9: 18529694 | 45722 | 9: 18552564 | 45724 | 9: 18645348 |
| 45726 | 9: 18717619 | 45727 | 9: 18731789 | 45729 | 9: 18755368 | 45731 | 9: 18791756 | 45732 | 9: 18791819 |
| 45733 | 9: 18791889 | 45738 | 9: 18936509 | 45743 | 9: 19046499 | 45745 | 9: 19139676 | 45753 | 9: 19435053 |
| 45776 | 9: 20235228 | 45779 | 9: 20303916 | 45816 | 9: 21552246 | 45824 | 9: 21653355 | 45833 | 9: 21833987 |
| 45835 | 9: 21852388 | 45840 | 9: 21940817 | 45846 | 9: 22080892 | 45848 | 9: 22133199 | 45865 | 9: 22573520 |
| 45867 | 9: 22594113 | 45874 | 9: 22795139 | 45879 | 9: 22958243 | 45889 | 9: 23151477 | 45890 | 9: 23173931 |
| 45894 | 9: 23218165 | 45898 | 9: 23339249 | 45900 | 9: 23430616 | 45901 | 9: 23441779 | 45904 | 9: 23456305 |
| 45906 | 9: 23472649 | 45909 | 9: 23546774 | 45915 | 9: 23672358 | 45925 | 9: 23857068 | 45932 | 9: 24029631 |
| 45937 | 9: 24145793 | 45943 | 9: 24238183 | 45951 | 9: 24426538 | 45970 | 9: 25002484 | 45980 | 9: 25350079 |
| 45981 | 9: 25353185 | 45989 | 9: 25530018 | 45997 | 9: 25755576 | 45999 | 9: 25785154 | 46003 | 9: 25878075 |
| 46010 | 9: 26028894 | 46015 | 9: 26134547 | 46018 | 9: 26208349 | 46028 | 9: 26467397 | 46039 | 9: 26838528 |
| 46040 | 9: 26868103 | 46041 | 9: 26898919 | 46048 | 9: 27183994 | 46054 | 9: 27249330 | 46057 | 9: 27304567 |
| 46058 | 9: 27317016 | 46061 | 9: 27408248 | 46071 | 9: 27587922 | 46077 | 9: 27702582 | 46094 | 9: 28136433 |
| 46108 | 9: 28431328 | 46121 | 9: 28644696 | 46127 | 9: 28779206 | 46165 | 9: 29391430 | 46177 | 9: 29530654 |
| 46179 | 9: 29544266 | 46183 | 9: 29615614 | 46205 | 9: 30230309 | 46207 | 9: 30256998 | 46208 | 9: 30257023 |
| 46209 | 9: 30257052 | 46212 | 9: 30289673 | 46219 | 9: 30469930 | 46224 | 9: 30615123 | 46227 | 9: 30674435 |
| 46229 | 9: 30739336 | 46237 | 9: 30892065 | 46239 | 9: 30932509 | 46240 | 9: 30932519 | 46241 | 9: 30932556 |
| 46247 | 9: 31029451 | 46249 | 9: 31141266 | 46252 | 9: 31227971 | 46253 | 9: 31227973 | 46255 | 9: 31253066 |
| 46261 | 9: 31394461 | 46271 | 9: 31555978 | 46272 | 9: 31588762 | 46273 | 9: 31588794 | 46275 | 9: 31725734 |
| 46283 | 9: 31984894 | 46290 | 9: 32052617 | 46291 | 9: 32063888 | 46292 | 9: 32063963 | 46296 | 9: 32225638 |
| 46300 | 9: 32284534 | 46301 | 9: 32291884 | 46307 | 9: 32437975 | 46308 | 9: 32501496 | 46312 | 9: 32638914 |
| 46315 | 9: 32720871 | 46342 | 9: 33372243 | 46349 | 9: 33488593 | 46350 | 9: 33496695 | 46356 | 9: 33730807 |
| 46357 | 9: 33736686 | 46358 | 9: 33736719 | 46365 | 9: 33922999 | 46376 | 9: 34234529 | 46377 | 9: 34239640 |
| 46383 | 9: 34398858 | 46405 | 9: 35059112 | 46422 | 9: 35505083 | 46424 | 9: 35630351 | 46428 | 9: 35723297 |
| 46429 | 9: 35723312 | 46430 | 9: 35723316 | 46431 | 9: 35723347 | 46432 | 9: 35723412 | 46438 | 9: 35923167 |
| 46445 | 9: 36108895 | 46446 | 9: 36118700 | 46448 | 9: 36196365 | 46457 | 9: 36321163 | 46464 | 9: 36515253 |
| 46466 | 9: 36617540 | 46468 | 9: 36686271 | 46470 | 9: 36765440 | 46475 | 9: 36837540 | 46482 | 9: 36952973 |
| 46485 | 9: 36987967 | 46495 | 9: 37162141 | 46500 | 9: 37248499 | 46501 | 9: 37262739 | 46504 | 9: 37446666 |
| 46518 | 9: 37786775 | 46532 | 9: 38042996 | 46538 | 9: 38200604 | 46540 | 9: 38214307 | 46541 | 9: 38222757 |
| 46542 | 9: 38234690 | 46548 | 9: 38345134 | 46549 | 9: 38346604 | 46550 | 9: 38349255 | 46551 | 9: 38361398 |
| 46555 | 9: 38439726 | 46556 | 9: 38458789 | 46558 | 9: 38513565 | 46560 | 9: 38544090 | 46562 | 9: 38561388 |
| 46568 | 9: 38630128 | 46575 | 9: 38725892 | 46578 | 9: 38828299 | 46585 | 9: 38890408 | 46589 | 9: 38943133 |
| 46590 | 9: 38963419 | 46603 | 9: 39377711 | 46610 | 9: 39610211 | 46611 | 9: 39633208 | 46612 | 9: 39637416 |
| 46613 | 9: 39638258 | 46614 | 9: 39639117 | 46616 | 9: 39648286 | 46622 | 9: 39764057 | 46623 | 9: 39784094 |
| 46624 | 9: 39793093 | 46627 | 9: 39807347 | 46628 | 9: 39807953 | 46629 | 9: 39811555 | 46634 | 9: 39934012 |
| 46635 | 9: 39956055 | 46638 | 9: 40007646 | 46646 | 9: 40343063 | 46647 | 9: 40346754 | 46649 | 9: 40351241 |
| 46650 | 9: 40353887 | 46651 | 9: 40359510 | 46652 | 9: 40394895 | 46653 | 9: 40405455 | 46658 | 9: 40559500 |
| 46664 | 9: 40689518 | 46670 | 9: 40792949 | 46673 | 9: 40849838 | 46674 | 9: 40851485 | 46675 | 9: 40876197 |
| 46676 | 9: 40920502 | 46677 | 9: 40928750 | 46678 | 9: 40936292 | 46679 | 9: 40949947 | 46680 | 9: 40960994 |
| 46681 | 9: 40986746 | 46684 | 9: 41057481 | 46690 | 9: 41209197 | 46698 | 9: 41519871 | 46702 | 9: 41630405 |
| 46705 | 9: 41713976 | 46712 | 9: 42008068 | 46714 | 9: 42049316 | 46715 | 9: 42066260 | 46716 | 9: 42073188 |
| 46721 | 9: 42199260 | 46722 | 9: 42202691 | 46723 | 9: 42206745 | 46724 | 9: 42207524 | 46726 | 9: 42218456 |
| 46738 | 9: 42539807 | 46741 | 9: 42738988 | 46742 | 9: 42751569 | 46743 | 9: 42754079 | 46751 | 9: 43079312 |
| 46752 | 9: 43096509 | 46754 | 9: 43117290 | 46755 | 9: 43117292 | 46756 | 9: 43117313 | 46757 | 9: 43117336 |
| 46758 | 9: 43117351 | 46759 | 9: 43155466 | 46760 | 9: 43155519 | 46761 | 9: 43155650 | 46766 | 9: 43352867 |
| 46779 | 9: 43685269 | 46787 | 9: 43948226 | 46803 | 9: 44341413 | 46817 | 9: 44599779 | 46820 | 9: 44637804 |
| 46826 | 9: 44854910 | 46842 | 9: 45505888 | 46845 | 9: 45550200 | 46868 | 9: 46443428 | 46877 | 9: 46667396 |
| 46883 | 9: 46847687 | 46895 | 9: 47304057 | 46923 | 9: 48075628 | 46930 | 9: 48336145 | 46939 | 9: 48787417 |
| 46954 | 9: 49224046 | 46955 | 9: 49225376 | 46982 | 9: 49910883 | 46995 | 9: 50277864 | 47007 | 9: 50567297 |
| 47008 | 9: 50567298 | 47009 | 9: 50567390 | 47012 | 9: 50646985 | 47015 | 9: 50694324 | 47019 | 9: 50797460 |
| 47059 | 9: 52046890 | 47080 | 9: 52645687 | 47088 | 9: 52787927 | 47093 | 9: 52848517 | 47095 | 9: 52861826 |
| 47120 | 9: 53314350 | 47139 | 9: 54071333 | 47143 | 9: 54193058 | 47146 | 9: 54279421 | 47153 | 9: 54522488 |
| 47161 | 9: 54675346 | 47181 | 9: 55237255 | 47192 | 9: 55526568 | 47193 | 9: 55526660 | 47194 | 9: 55529308 |
| 47228 | 9: 56645296 | 47230 | 9: 56687693 | 47234 | 9: 56760440 | 47236 | 9: 56787195 | 47245 | 9: 57057433 |
| 47259 | 9: 57574987 | 47260 | 9: 57574995 | 47262 | 9: 57634613 | 47272 | 9: 57793857 | 47276 | 9: 57849001 |
| 47284 | 9: 58149441 | 47287 | 9: 58311080 | 47288 | 9: 58333810 | 47293 | 9: 58457036 | 47301 | 9: 58652669 |
| 47314 | 9: 59159045 | 47351 | 9: 59715217 | 47353 | 9: 59771120 | 47355 | 9: 59857183 | 47362 | 9: 60072130 |
| 47364 | 9: 60134972 | 47367 | 9: 60247427 | 47369 | 9: 60291706 | 47372 | 9: 60358510 | 47377 | 9: 60481368 |
| 47380 | 9: 60536321 | 47383 | 9: 60631920 | 47388 | 9: 60745480 | 47393 | 9: 60896389 | 47394 | 9: 60903838 |
| 47397 | 9: 60928250 | 47398 | 9: 60928272 | 47400 | 9: 60929060 | 47401 | 9: 60930496 | 47402 | 9: 60932205 |
| 47403 | 9: 60936701 | 47406 | 9: 61034023 | 47420 | 9: 61625663 | 47428 | 9: 61883689 | 47437 | 9: 62153095 |
| 47438 | 9: 62160605 | 47441 | 9: 62239934 | 47444 | 9: 62407115 | 47446 | 9: 62441325 | 47448 | 9: 62541808 |
| 47449 | 9: 62541844 | 47450 | 9: 62541925 | 47451 | 9: 62569285 | 47453 | 9: 62596000 | 47475 | 9: 63213406 |
| 47481 | 9: 63334601 | 47482 | 9: 63334709 | 47483 | 9: 63334737 | 47488 | 9: 63564229 | 47490 | 9: 63662723 |
| 47495 | 9: 63879901 | 47505 | 9: 64262650 | 47522 | 9: 64653298 | 47523 | 9: 64675425 | 47526 | 9: 64806879 |
| 47527 | 9: 64818979 | 47536 | 9: 65014222 | 47537 | 9: 65014886 | 47538 | 9: 65015900 | 47539 | 9: 65016625 |
| 47540 | 9: 65017299 | 47541 | 9: 65017970 | 47542 | 9: 65018705 | 47543 | 9: 65020386 | 47544 | 9: 65022012 |
| 47545 | 9: 65022960 | 47546 | 9: 65024531 | 47547 | 9: 65025971 | 47548 | 9: 65026593 | 47549 | 9: 65027537 |
| 47550 | 9: 65029266 | 47551 | 9: 65031641 | 47552 | 9: 65033856 | 47553 | 9: 65034695 | 47554 | 9: 65037061 |
| 47555 | 9: 65038847 | 47556 | 9: 65039895 | 47557 | 9: 65041060 | 47558 | 9: 65041925 | 47559 | 9: 65046945 |
| 47560 | 9: 65048753 | 47561 | 9: 65051331 | 47562 | 9: 65052345 | 47564 | 9: 65062111 | 47565 | 9: 65063669 |
| 47566 | 9: 65065713 | 47567 | 9: 65069167 | 47568 | 9: 65072452 | 47569 | 9: 65074602 | 47570 | 9: 65075729 |
| 47571 | 9: 65076561 | 47572 | 9: 65077195 | 47573 | 9: 65077758 | 47574 | 9: 65078630 | 47575 | 9: 65118677 |
| 47576 | 9: 65123323 | 47578 | 9: 65179118 | 47579 | 9: 65235666 | 47580 | 9: 65243193 | 47581 | 9: 65244259 |
| 47586 | 9: 65383024 | 47589 | 9: 65443065 | 47590 | 9: 65446545 | 47591 | 9: 65478206 | 47592 | 9: 65483403 |
| 47598 | 9: 65568211 | 47599 | 9: 65570457 | 47600 | 9: 65572272 | 47602 | 9: 65604455 | 47609 | 9: 65891235 |
| 47612 | 9: 65971970 | 47615 | 9: 66011528 | 47638 | 9: 66597468 | 47648 | 9: 66989120 | 47657 | 9: 67225760 |
| 47663 | 9: 67332887 | 47668 | 9: 67459980 | 47681 | 9: 67796936 | 47682 | 9: 67823147 | 47686 | 9: 67954748 |
| 47705 | 9: 68370513 | 47708 | 9: 68427995 | 47720 | 9: 68912666 | 47721 | 9: 68917528 | 47729 | 9: 69060023 |
| 47730 | 9: 69093647 | 47731 | 9: 69104702 | 47732 | 9: 69114502 | 47738 | 9: 69297159 | 47741 | 9: 69407676 |
| 47750 | 9: 69583761 | 47751 | 9: 69599853 | 47761 | 9: 69896845 | 47767 | 9: 70012144 | 47777 | 9: 70295013 |
| 47779 | 9: 70342326 | 47780 | 9: 70347483 | 47786 | 9: 70409386 | 47787 | 9: 70409422 | 47789 | 9: 70429923 |
| 47797 | 9: 70751662 | 47798 | 9: 70753720 | 47805 | 9: 70887910 | 47809 | 9: 71049805 | 47811 | 9: 71060333 |
| 47813 | 9: 71107122 | 47815 | 9: 71116611 | 47820 | 9: 71251152 | 47822 | 9: 71292789 | 47824 | 9: 71344053 |
| 47825 | 9: 71356859 | 47826 | 9: 71384334 | 47828 | 9: 71407368 | 47829 | 9: 71445600 | 47831 | 9: 71492315 |
| 47834 | 9: 71512303 | 47835 | 9: 71513737 | 47840 | 9: 71612772 | 47844 | 9: 71651985 | 47848 | 9: 71736589 |
| 47849 | 9: 71761625 | 47864 | 9: 72304039 | 47867 | 9: 72384960 | 47877 | 9: 72575284 | 47882 | 9: 72722658 |
| 47883 | 9: 72725124 | 47891 | 9: 72888193 | 47892 | 9: 72973276 | 47893 | 9: 72989037 | 47895 | 9: 73138567 |
| 47897 | 9: 73175040 | 47902 | 9: 73223596 | 47903 | 9: 73244841 | 47915 | 9: 73594271 | 47920 | 9: 73701336 |
| 47921 | 9: 73701377 | 47937 | 9: 74031324 | 47939 | 9: 74043724 | 47948 | 9: 74315428 | 47955 | 9: 74575135 |
| 47957 | 9: 74617923 | 47959 | 9: 74622543 | 47974 | 9: 75094490 | 47975 | 9: 75114725 | 47979 | 9: 75261135 |
| 47984 | 9: 75482552 | 47987 | 9: 75505924 | 47991 | 9: 75564777 | 47995 | 9: 75627446 | 47999 | 9: 75748546 |
| 48000 | 9: 75749661 | 48001 | 9: 75754806 | 48002 | 9: 75754987 | 48014 | 9: 76061643 | 48016 | 9: 76103605 |
| 48022 | 9: 76314846 | 48023 | 9: 76319841 | 48038 | 9: 76900400 | 48041 | 9: 76954502 | 48042 | 9: 76961448 |
| 48045 | 9: 76972318 | 48048 | 9: 77037622 | 48049 | 9: 77047736 | 48050 | 9: 77049274 | 48058 | 9: 77255407 |
| 48063 | 9: 77401724 | 48071 | 9: 77497521 | 48150 | 9: 78018701 | 48157 | 9: 78383301 | 48163 | 9: 78618960 |
| 48166 | 9: 78653185 | 48173 | 9: 78856788 | 48176 | 9: 78984268 | 48192 | 9: 79629564 | 48204 | 9: 79900857 |
| 48206 | 9: 79927447 | 48209 | 9: 79992577 | 48211 | 9: 79999363 | 48220 | 9: 80143039 | 48222 | 9: 80223447 |
| 48226 | 9: 80281892 | 48235 | 9: 80379126 | 48251 | 9: 80913419 | 48252 | 9: 80926042 | 48253 | 9: 80927048 |
| 48254 | 9: 80927101 | 48255 | 9: 80935101 | 48256 | 9: 80938115 | 48260 | 9: 80960455 | 48281 | 9: 81389583 |
| 48286 | 9: 81502463 | 48289 | 9: 81518292 | 48299 | 9: 81745339 | 48304 | 9: 81841021 | 48317 | 9: 82097477 |
| 48318 | 9: 82097512 | 48319 | 9: 82100313 | 48337 | 9: 82534749 | 48362 | 9: 82854874 | 48364 | 9: 82923714 |
| 48369 | 9: 83119086 | 48374 | 9: 83237503 | 48377 | 9: 83309469 | 48391 | 9: 83641203 | 48392 | 9: 83672779 |
| 48399 | 9: 83841026 | 48400 | 9: 83857767 | 48418 | 9: 84276446 | 48447 | 9: 85144864 | 48452 | 9: 85201593 |
| 48455 | 9: 85222343 | 48457 | 9: 85277103 | 48474 | 9: 85565902 | 48476 | 9: 85582831 | 48477 | 9: 85582845 |
| 48478 | 9: 85582856 | 48479 | 9: 85582930 | 48485 | 9: 85757204 | 48486 | 9: 85777430 | 48487 | 9: 85777435 |
| 48488 | 9: 85777468 | 48489 | 9: 85777555 | 48492 | 9: 85801137 | 48494 | 9: 85812059 | 48500 | 9: 85926846 |
| 48507 | 9: 86110491 | 48514 | 9: 86329047 | 48532 | 9: 86823737 | 48534 | 9: 86935158 | 48537 | 9: 86963116 |
| 48539 | 9: 86989548 | 48541 | 9: 87028872 | 48556 | 9: 87348985 | 48561 | 9: 87384465 | 48568 | 9: 87494785 |
| 48570 | 9: 87503445 | 48571 | 9: 87504577 | 48572 | 9: 87542588 | 48573 | 9: 87544854 | 48593 | 9: 87878916 |
| 48595 | 9: 87890519 | 48596 | 9: 87917511 | 48598 | 9: 87929924 | 48599 | 9: 87952953 | 48600 | 9: 87962089 |
| 48601 | 9: 87962734 | 48603 | 9: 87966017 | 48604 | 9: 87966917 | 48605 | 9: 87975738 | 48610 | 9: 88087724 |
| 48612 | 9: 88140632 | 48613 | 9: 88160188 | 48626 | 9: 88569166 | 48628 | 9: 88636603 | 48629 | 9: 88636618 |
| 48630 | 9: 88636634 | 48635 | 9: 88732430 | 48644 | 9: 88925002 | 48646 | 9: 88943609 | 48654 | 9: 89094468 |
| 48656 | 9: 89166354 | 48658 | 9: 89189099 | 48666 | 9: 89326613 | 48667 | 9: 89345202 | 48668 | 9: 89346130 |
| 48670 | 9: 89356829 | 48671 | 9: 89373227 | 48672 | 9: 89380245 | 48673 | 9: 89387102 | 48674 | 9: 89399914 |
| 48676 | 9: 89410907 | 48677 | 9: 89417136 | 48678 | 9: 89423178 | 48680 | 9: 89436296 | 48681 | 9: 89443866 |
| 48682 | 9: 89445782 | 48683 | 9: 89445926 | 48684 | 9: 89447805 | 48685 | 9: 89454999 | 48688 | 9: 89471837 |
| 48690 | 9: 89496573 | 48698 | 9: 89664367 | 48699 | 9: 89708244 | 48700 | 9: 89708807 | 48709 | 9: 89919288 |
| 48733 | 9: 90540018 | 48744 | 9: 90795244 | 48745 | 9: 90796860 | 48750 | 9: 90844111 | 48751 | 9: 90845595 |
| 48752 | 9: 90845718 | 48762 | 9: 91108815 | 48767 | 9: 91144245 | 48776 | 9: 91423091 | 48783 | 9: 91591725 |
| 48784 | 9: 91611257 | 48786 | 9: 91670541 | 48789 | 9: 91728675 | 48790 | 9: 91734111 | 48794 | 9: 91799932 |
| 48795 | 9: 91808084 | 48802 | 9: 92017600 | 48804 | 9: 92051007 | 48808 | 9: 92146096 | 48809 | 9: 92173136 |
| 48823 | 9: 92434720 | 48827 | 9: 92618757 | 48831 | 9: 92719541 | 48833 | 9: 92726807 | 48834 | 9: 92726908 |
| 48835 | 9: 92732978 | 48844 | 9: 92935922 | 48848 | 9: 93101440 | 48860 | 9: 93255745 | 48864 | 9: 93329746 |
| 48865 | 9: 93335995 | 48868 | 9: 93434039 | 48869 | 9: 93439818 | 48871 | 9: 93544670 | 48872 | 9: 93562913 |
| 48873 | 9: 93583179 | 48874 | 9: 93589365 | 48876 | 9: 93593753 | 48878 | 9: 93648481 | 48881 | 9: 93756818 |
| 48882 | 9: 93761851 | 48888 | 9: 93864628 | 48899 | 9: 94037282 | 48902 | 9: 94059439 | 48903 | 9: 94075786 |
| 48908 | 9: 94239134 | 48909 | 9: 94242901 | 48916 | 9: 94279436 | 48925 | 9: 94506574 | 48926 | 9: 94511166 |
| 48928 | 9: 94549002 | 48929 | 9: 94552504 | 48930 | 9: 94571641 | 48938 | 9: 94770144 | 48943 | 9: 94849176 |
| 48951 | 9: 95011385 | 48952 | 9: 95011440 | 48954 | 9: 95075297 | 48957 | 9: 95117095 | 48958 | 9: 95119909 |
| 48959 | 9: 95120912 | 48960 | 9: 95120918 | 48961 | 9: 95121102 | 48963 | 9: 95137543 | 48965 | 9: 95146949 |
| 48966 | 9: 95149713 | 48967 | 9: 95151479 | 48968 | 9: 95151880 | 48969 | 9: 95154305 | 48970 | 9: 95159375 |
| 48971 | 9: 95171473 | 48972 | 9: 95239467 | 48975 | 9: 95257977 | 48996 | 9: 95960164 | 48997 | 9: 95961292 |
| 48998 | 9: 95964766 | 48999 | 9: 95965222 | 49013 | 9: 96262623 | 49016 | 9: 96335077 | 49018 | 9: 96352203 |
| 49020 | 9: 96364316 | 49021 | 9: 96365825 | 49023 | 9: 96394133 | 49026 | 9: 96454587 | 49027 | 9: 96459992 |
| 49028 | 9: 96460776 | 49029 | 9: 96462068 | 49030 | 9: 96464377 | 49031 | 9: 96464974 | 49032 | 9: 96465729 |
| 49033 | 9: 96467828 | 49034 | 9: 96471984 | 49035 | 9: 96474365 | 49036 | 9: 96475139 | 49037 | 9: 96477053 |
| 49038 | 9: 96478790 | 49039 | 9: 96480951 | 49040 | 9: 96482190 | 49041 | 9: 96487368 | 49045 | 9: 96575319 |
| 49047 | 9: 96602946 | 49050 | 9: 96636351 | 49064 | 9: 96811911 | 49067 | 9: 96862976 | 49068 | 9: 96865435 |
| 49071 | 9: 96875852 | 49072 | 9: 96886282 | 49078 | 9: 97002454 | 49079 | 9: 97005408 | 49080 | 9: 97006623 |
| 49107 | 9: 97443586 | 49108 | 9: 97443633 | 49112 | 9: 97554130 | 49114 | 9: 97571949 | 49117 | 9: 97636004 |
| 49122 | 9: 97688394 | 49125 | 9: 97734208 | 49128 | 9: 97781409 | 49129 | 9: 97814093 | 49132 | 9: 97896936 |
| 49140 | 9: 97952202 | 49142 | 9: 97987774 | 49146 | 9: 98072257 | 49147 | 9: 98090254 | 49149 | 9: 98186273 |
| 49152 | 9: 98271044 | 49162 | 9: 98520046 | 49169 | 9: 98643289 | 49173 | 9: 98706036 | 49174 | 9: 98706126 |
| 49177 | 9: 98800653 | 49185 | 9: 99049511 | 49197 | 9: 99258284 | 49201 | 9: 99320226 | 49202 | 9: 99340641 |
| 49203 | 9: 99363193 | 49205 | 9: 99375752 | 49213 | 9: 99564669 | 49223 | 9: 99707846 | 49233 | 9: 99913842 |
| 49238 | 9: 100020368 | 49239 | 9: 100037134 | 49248 | 9: 100222700 | 49254 | 9: 100288205 | 49264 | 9: 100469286 |
| 49278 | 9: 100659300 | 49294 | 9: 101011242 | 49295 | 9: 101027872 | 49296 | 9: 101037743 | 49299 | 9: 101107041 |
| 49302 | 9: 101154740 | 49305 | 9: 101206979 | 49311 | 9: 101309902 | 49313 | 9: 101329946 | 49314 | 9: 101388778 |
| 49315 | 9: 101404988 | 49320 | 9: 101473236 | 49325 | 9: 101520399 | 49330 | 9: 101680647 | 49341 | 9: 101799308 |
| 49351 | 9: 101941022 | 49353 | 9: 101969310 | 49354 | 9: 101969389 | 49355 | 9: 101970659 | 49356 | 9: 101970691 |
| 49357 | 9: 101971384 | 49358 | 9: 101971424 | 49359 | 9: 101976339 | 49360 | 9: 101977590 | 49361 | 9: 101977632 |
| 49362 | 9: 101977719 | 49363 | 9: 101977742 | 49364 | 9: 101984019 | 49365 | 9: 101984050 | 49366 | 9: 101984250 |
| 49367 | 9: 102004205 | 49368 | 9: 102034495 | 49369 | 9: 102034523 | 49370 | 9: 102034580 | 49371 | 9: 102034582 |
| 49373 | 9: 102053353 | 49375 | 9: 102065589 | 49376 | 9: 102121235 | 49377 | 9: 102127863 | 49380 | 9: 102237624 |
| 49390 | 9: 102348030 | 49391 | 9: 102364770 | 49400 | 9: 102589137 | 49402 | 9: 102618152 | 49403 | 9: 102619914 |
| 49404 | 9: 102620509 | 49405 | 9: 102622882 | 49409 | 9: 102654533 | 49414 | 9: 102687424 | 49416 | 9: 102706741 |
| 49417 | 9: 102710336 | 49422 | 9: 102795356 | 49426 | 9: 102898617 | 49427 | 9: 102898674 | 49438 | 9: 103153314 |
| 49439 | 9: 103215814 | 49446 | 9: 103329162 | 49447 | 9: 103333336 | 49448 | 9: 103336781 | 49457 | 9: 103493999 |
| 49459 | 9: 103575109 | 49460 | 9: 103582734 | 49473 | 9: 103926483 | 49474 | 9: 103926928 | 49475 | 9: 103927077 |
| 49476 | 9: 103969118 | 49477 | 9: 104003359 | 49485 | 9: 104113993 | 49489 | 9: 104355196 | 49490 | 9: 104355303 |
| 49491 | 9: 104356588 | 49492 | 9: 104356685 | 49494 | 9: 104376710 | 49501 | 10: 84875 | 49519 | 10: 695399 |
| 49526 | 10: 769669 | 49544 | 10: 1257872 | 49554 | 10: 1412883 | 49570 | 10: 1805121 | 49574 | 10: 1814057 |
| 49576 | 10: 1856119 | 49578 | 10: 1881286 | 49579 | 10: 1881323 | 49593 | 10: 2147400 | 49602 | 10: 2351254 |
| 49605 | 10: 2373566 | 49626 | 10: 2903794 | 49627 | 10: 2903827 | 49629 | 10: 2929612 | 49630 | 10: 2932512 |
| 49647 | 10: 3209297 | 49674 | 10: 3744198 | 49675 | 10: 3744214 | 49677 | 10: 3772515 | 49678 | 10: 3774094 |
| 49679 | 10: 3802315 | 49691 | 10: 4074788 | 49692 | 10: 4104638 | 49705 | 10: 4448386 | 49707 | 10: 4468373 |
| 49709 | 10: 4484450 | 49711 | 10: 4554748 | 49716 | 10: 4604295 | 49720 | 10: 4638128 | 49726 | 10: 4828321 |
| 49727 | 10: 4829589 | 49732 | 10: 5012847 | 49735 | 10: 5111561 | 49755 | 10: 5476055 | 49758 | 10: 5506479 |
| 49775 | 10: 5547565 | 49776 | 10: 5550036 | 49777 | 10: 5557416 | 49781 | 10: 5609083 | 49797 | 10: 6019241 |
| 49799 | 10: 6059501 | 49802 | 10: 6133685 | 49812 | 10: 6396765 | 49816 | 10: 6454997 | 49818 | 10: 6473524 |
| 49820 | 10: 6505409 | 49823 | 10: 6590721 | 49826 | 10: 6677707 | 49857 | 10: 7516833 | 49859 | 10: 7543493 |
| 49862 | 10: 7600499 | 49873 | 10: 7761962 | 49880 | 10: 7901116 | 49884 | 10: 8008379 | 49898 | 10: 8326560 |
| 49905 | 10: 8450920 | 49907 | 10: 8478523 | 49911 | 10: 8510644 | 49912 | 10: 8510646 | 49914 | 10: 8553549 |
| 49915 | 10: 8553909 | 49920 | 10: 8708761 | 49922 | 10: 8777935 | 49926 | 10: 8877008 | 49931 | 10: 9028658 |
| 49934 | 10: 9110701 | 49955 | 10: 9671703 | 49956 | 10: 9680242 | 49958 | 10: 9697568 | 49963 | 10: 9786271 |
| 49968 | 10: 9804614 | 49979 | 10: 10147987 | 49980 | 10: 10172215 | 49983 | 10: 10207865 | 49985 | 10: 10220078 |
| 49990 | 10: 10286771 | 49992 | 10: 10393696 | 49993 | 10: 10400749 | 49997 | 10: 10519536 | 49999 | 10: 10536820 |
| 50000 | 10: 10537343 | 50001 | 10: 10611527 | 50002 | 10: 10633817 | 50005 | 10: 10700036 | 50006 | 10: 10706615 |
| 50007 | 10: 10740773 | 50008 | 10: 10740870 | 50009 | 10: 10740872 | 50010 | 10: 10742093 | 50012 | 10: 10745459 |
| 50020 | 10: 10855774 | 50025 | 10: 10952491 | 50026 | 10: 10983147 | 50028 | 10: 10992296 | 50031 | 10: 11037910 |
| 50037 | 10: 11124874 | 50041 | 10: 11194365 | 50042 | 10: 11199580 | 50043 | 10: 11214627 | 50044 | 10: 11214653 |
| 50051 | 10: 11459762 | 50052 | 10: 11480188 | 50053 | 10: 11484963 | 50054 | 10: 11485435 | 50055 | 10: 11533676 |
| 50062 | 10: 11746501 | 50071 | 10: 11984525 | 50072 | 10: 11993432 | 50075 | 10: 12037788 | 50077 | 10: 12049463 |
| 50078 | 10: 12049470 | 50080 | 10: 12056720 | 50081 | 10: 12058675 | 50082 | 10: 12060333 | 50085 | 10: 12193255 |
| 50087 | 10: 12210976 | 50092 | 10: 12363067 | 50096 | 10: 12446219 | 50104 | 10: 12563184 | 50108 | 10: 12662259 |
| 50112 | 10: 12776928 | 50118 | 10: 13028042 | 50125 | 10: 13148280 | 50129 | 10: 13195695 | 50130 | 10: 13201885 |
| 50137 | 10: 13233050 | 50141 | 10: 13251806 | 50146 | 10: 13290512 | 50155 | 10: 13433297 | 50159 | 10: 13500606 |
| 50160 | 10: 13521494 | 50163 | 10: 13603249 | 50164 | 10: 13604834 | 50184 | 10: 13904093 | 50186 | 10: 13947394 |
| 50187 | 10: 13947449 | 50188 | 10: 13953167 | 50191 | 10: 14062089 | 50201 | 10: 14318815 | 50203 | 10: 14387176 |
| 50204 | 10: 14387540 | 50216 | 10: 14685085 | 50230 | 10: 15045027 | 50231 | 10: 15055444 | 50239 | 10: 15212747 |
| 50241 | 10: 15253352 | 50242 | 10: 15253401 | 50244 | 10: 15306720 | 50250 | 10: 15382216 | 50251 | 10: 15382286 |
| 50254 | 10: 15491085 | 50261 | 10: 15626947 | 50268 | 10: 15765399 | 50269 | 10: 15766293 | 50270 | 10: 15766923 |
| 50271 | 10: 15768048 | 50272 | 10: 15777260 | 50274 | 10: 15872387 | 50275 | 10: 15872397 | 50276 | 10: 15878690 |
| 50277 | 10: 15887011 | 50278 | 10: 15887086 | 50279 | 10: 15888077 | 50280 | 10: 15888157 | 50286 | 10: 15986933 |
| 50287 | 10: 15988001 | 50293 | 10: 16160207 | 50294 | 10: 16160289 | 50295 | 10: 16160311 | 50300 | 10: 16292572 |
| 50303 | 10: 16351611 | 50309 | 10: 16477102 | 50311 | 10: 16505051 | 50315 | 10: 16579579 | 50317 | 10: 16615178 |
| 50318 | 10: 16616715 | 50319 | 10: 16622209 | 50321 | 10: 16657010 | 50323 | 10: 16689144 | 50325 | 10: 16708640 |
| 50329 | 10: 16760750 | 50330 | 10: 16783563 | 50332 | 10: 16840169 | 50333 | 10: 16840192 | 50334 | 10: 16840220 |
| 50346 | 10: 17026997 | 50358 | 10: 17327263 | 50362 | 10: 17375623 | 50364 | 10: 17405609 | 50372 | 10: 17576175 |
| 50380 | 10: 17746328 | 50393 | 10: 18053005 | 50409 | 10: 18204972 | 50413 | 10: 18326097 | 50417 | 10: 18459800 |
| 50429 | 10: 18655176 | 50430 | 10: 18656419 | 50440 | 10: 19055444 | 50441 | 10: 19055478 | 50444 | 10: 19108089 |
| 50445 | 10: 19108125 | 50446 | 10: 19108146 | 50447 | 10: 19108188 | 50448 | 10: 19108578 | 50449 | 10: 19108623 |
| 50450 | 10: 19108629 | 50463 | 10: 19296017 | 50473 | 10: 19582204 | 50483 | 10: 19757013 | 50484 | 10: 19757066 |
| 50485 | 10: 19757109 | 50492 | 10: 19887391 | 50493 | 10: 19887438 | 50494 | 10: 19887463 | 50495 | 10: 19887464 |
| 50496 | 10: 19887506 | 50497 | 10: 19906833 | 50502 | 10: 20032774 | 50522 | 10: 20511602 | 50523 | 10: 20517856 |
| 50531 | 10: 20716836 | 50533 | 10: 20746648 | 50534 | 10: 20749216 | 50535 | 10: 20765182 | 50538 | 10: 20798013 |
| 50539 | 10: 20800521 | 50540 | 10: 20823031 | 50542 | 10: 20851467 | 50543 | 10: 20854079 | 50545 | 10: 20866367 |
| 50546 | 10: 20866872 | 50547 | 10: 20878828 | 50548 | 10: 20887914 | 50549 | 10: 20905201 | 50550 | 10: 20905351 |
| 50551 | 10: 20906045 | 50552 | 10: 20922221 | 50555 | 10: 20962445 | 50556 | 10: 20969179 | 50557 | 10: 20971568 |
| 50558 | 10: 20980278 | 50559 | 10: 20992105 | 50561 | 10: 21041241 | 50564 | 10: 21086459 | 50570 | 10: 21193718 |
| 50581 | 10: 21392700 | 50586 | 10: 21482874 | 50587 | 10: 21494322 | 50588 | 10: 21496846 | 50590 | 10: 21512555 |
| 50602 | 10: 21928460 | 50604 | 10: 21943044 | 50606 | 10: 21954776 | 50607 | 10: 21955205 | 50608 | 10: 21961681 |
| 50609 | 10: 21969630 | 50610 | 10: 21969644 | 50611 | 10: 21969862 | 50612 | 10: 21970634 | 50613 | 10: 21972554 |
| 50620 | 10: 22271591 | 50629 | 10: 22389791 | 50640 | 10: 22796412 | 50644 | 10: 23008980 | 50645 | 10: 23058608 |
| 50685 | 10: 25836315 | 50687 | 10: 25842537 | 50689 | 10: 25851554 | 50707 | 10: 26370686 | 50712 | 10: 26442221 |
| 50716 | 10: 26521248 | 50719 | 10: 26552273 | 50720 | 10: 26552368 | 50725 | 10: 26660705 | 50726 | 10: 26664869 |
| 50728 | 10: 26699741 | 50733 | 10: 26735536 | 50739 | 10: 26806421 | 50744 | 10: 26852657 | 50747 | 10: 26914900 |
| 50749 | 10: 26936311 | 50751 | 10: 26971279 | 50752 | 10: 26979102 | 50753 | 10: 26990817 | 50755 | 10: 27045849 |
| 50756 | 10: 27045893 | 50759 | 10: 27104985 | 50760 | 10: 27110316 | 50761 | 10: 27110398 | 50767 | 10: 27219754 |
| 50768 | 10: 27219761 | 50770 | 10: 27257251 | 50773 | 10: 27354015 | 50774 | 10: 27354260 | 50782 | 10: 27629698 |
| 50786 | 10: 27668710 | 50787 | 10: 27668712 | 50788 | 10: 27668749 | 50805 | 10: 28336043 | 50818 | 10: 28717710 |
| 50822 | 10: 28777408 | 50823 | 10: 28777482 | 50824 | 10: 28777483 | 50825 | 10: 28777517 | 50826 | 10: 28816832 |
| 50828 | 10: 28830145 | 50829 | 10: 28830820 | 50830 | 10: 28831951 | 50831 | 10: 28837592 | 50834 | 10: 28870874 |
| 50837 | 10: 28920327 | 50838 | 10: 28921627 | 50839 | 10: 28922441 | 50841 | 10: 28924088 | 50842 | 10: 28924117 |
| 50846 | 10: 28936523 | 50847 | 10: 28936611 | 50849 | 10: 28939012 | 50851 | 10: 28941456 | 50852 | 10: 28941564 |
| 50853 | 10: 28941645 | 50854 | 10: 28967860 | 50855 | 10: 28967993 | 50865 | 10: 29192994 | 50867 | 10: 29207986 |
| 50875 | 10: 29370574 | 50877 | 10: 29394079 | 50879 | 10: 29415078 | 50880 | 10: 29434816 | 50883 | 10: 29489538 |
| 50887 | 10: 29551880 | 50894 | 10: 29683783 | 50908 | 10: 30087181 | 50918 | 10: 30456072 | 50927 | 10: 30882706 |
| 50929 | 10: 31026079 | 50943 | 10: 31383428 | 50944 | 10: 31395880 | 50946 | 10: 31421937 | 50948 | 10: 31465645 |
| 50950 | 10: 31539600 | 50965 | 10: 31989230 | 50975 | 10: 32311663 | 50987 | 10: 32581894 | 50990 | 10: 32646966 |
| 50993 | 10: 32695364 | 50996 | 10: 32766762 | 51016 | 10: 33233733 | 51023 | 10: 33473275 | 51025 | 10: 33520438 |
| 51038 | 10: 33775393 | 51040 | 10: 33792624 | 51041 | 10: 33803176 | 51052 | 10: 34100470 | 51054 | 10: 34214726 |
| 51056 | 10: 34306045 | 51058 | 10: 34339538 | 51062 | 10: 34390466 | 51063 | 10: 34399556 | 51065 | 10: 34436191 |
| 51066 | 10: 34441644 | 51067 | 10: 34463264 | 51070 | 10: 34502888 | 51078 | 10: 34709456 | 51079 | 10: 34711929 |
| 51084 | 10: 34865113 | 51088 | 10: 34924089 | 51101 | 10: 35135295 | 51104 | 10: 35165655 | 51105 | 10: 35167815 |
| 51107 | 10: 35180569 | 51109 | 10: 35184337 | 51111 | 10: 35210635 | 51112 | 10: 35210945 | 51113 | 10: 35213913 |
| 51114 | 10: 35216558 | 51117 | 10: 35370763 | 51128 | 10: 35779938 | 51129 | 10: 35856231 | 51131 | 10: 35906682 |
| 51132 | 10: 35916585 | 51135 | 10: 35975249 | 51137 | 10: 35982115 | 51143 | 10: 36077032 | 51144 | 10: 36131193 |
| 51151 | 10: 36412382 | 51153 | 10: 36441443 | 51154 | 10: 36441686 | 51166 | 10: 36836677 | 51170 | 10: 36919070 |
| 51172 | 10: 36945666 | 51173 | 10: 36951207 | 51185 | 10: 37239881 | 51188 | 10: 37341433 | 51198 | 10: 37638488 |
| 51212 | 10: 37805187 | 51214 | 10: 37904587 | 51218 | 10: 37944601 | 51229 | 10: 38333207 | 51231 | 10: 38393603 |
| 51236 | 10: 38488614 | 51245 | 10: 38701360 | 51246 | 10: 38753237 | 51267 | 10: 39292295 | 51269 | 10: 39363047 |
| 51271 | 10: 39364510 | 51273 | 10: 39377386 | 51303 | 10: 40330913 | 51306 | 10: 40373055 | 51307 | 10: 40382252 |
| 51308 | 10: 40389854 | 51309 | 10: 40408498 | 51314 | 10: 40556907 | 51321 | 10: 40717249 | 51360 | 10: 41919935 |
| 51369 | 10: 42252004 | 51393 | 10: 42819454 | 51397 | 10: 42948816 | 51406 | 10: 43236868 | 51413 | 10: 43488771 |
| 51425 | 10: 43985352 | 51432 | 10: 44080128 | 51437 | 10: 44252723 | 51446 | 10: 44448699 | 51451 | 10: 44583693 |
| 51456 | 10: 44658796 | 51457 | 10: 44687920 | 51470 | 10: 44966040 | 51472 | 10: 45022368 | 51476 | 10: 45222091 |
| 51482 | 10: 45334197 | 51483 | 10: 45334223 | 51484 | 10: 45334295 | 51485 | 10: 45383083 | 51486 | 10: 45385249 |
| 51487 | 10: 45407393 | 51488 | 10: 45421821 | 51492 | 10: 45548786 | 51502 | 10: 45782999 | 51504 | 10: 45794472 |
| 51505 | 10: 45797020 | 51507 | 10: 45802351 | 51509 | 10: 45807766 | 51510 | 10: 45825756 | 51511 | 10: 45825757 |
| 51512 | 10: 45825760 | 51513 | 10: 45852804 | 51515 | 10: 45888410 | 51517 | 10: 45962441 | 51535 | 10: 46450572 |
| 51552 | 10: 46711744 | 51556 | 10: 46771999 | 51565 | 10: 47008189 | 51572 | 10: 47133365 | 51574 | 10: 47141503 |
| 51576 | 10: 47158682 | 51580 | 10: 47240491 | 51585 | 10: 47306453 | 51589 | 10: 47384963 | 51590 | 10: 47384967 |
| 51598 | 10: 47600619 | 51601 | 10: 47645531 | 51605 | 10: 47674190 | 51608 | 10: 47753050 | 51614 | 10: 48040907 |
| 51632 | 10: 48370815 | 51634 | 10: 48417884 | 51644 | 10: 48589378 | 51646 | 10: 48589442 | 51648 | 10: 48668493 |
| 51650 | 10: 48743669 | 51660 | 10: 49108347 | 51661 | 10: 49132750 | 51668 | 10: 49271034 | 51675 | 10: 49350595 |
| 51677 | 10: 49377221 | 51678 | 10: 49377286 | 51681 | 10: 49419982 | 51693 | 10: 49601513 | 51703 | 10: 49841256 |
| 51704 | 10: 49841837 | 51705 | 10: 49858694 | 51715 | 10: 50107685 | 51734 | 10: 50489426 | 51739 | 10: 50557454 |
| 51740 | 10: 50582984 | 51746 | 10: 50846288 | 51749 | 10: 51020894 | 51752 | 10: 51062674 | 51757 | 10: 51161988 |
| 51758 | 10: 51163453 | 51761 | 10: 51186753 | 51771 | 10: 51429834 | 51772 | 10: 51436172 | 51776 | 10: 51594174 |
| 51786 | 10: 51771324 | 51787 | 10: 51772122 | 51798 | 10: 52037658 | 51799 | 10: 52053671 | 51807 | 10: 52239885 |
| 51808 | 10: 52247304 | 51818 | 10: 52549691 | 51820 | 10: 52577136 | 51821 | 10: 52644574 | 51826 | 10: 52759280 |
| 51827 | 10: 52770881 | 51841 | 10: 52889989 | 51843 | 10: 52975439 | 51852 | 10: 53392084 | 51855 | 10: 53503163 |
| 51856 | 10: 53566836 | 51857 | 10: 53573889 | 51859 | 10: 53599523 | 51870 | 10: 54084338 | 51871 | 10: 54097737 |
| 51873 | 10: 54151043 | 51874 | 10: 54166847 | 51879 | 10: 54206436 | 51880 | 10: 54223827 | 51885 | 10: 54381985 |
| 51897 | 10: 54587863 | 51900 | 10: 54728840 | 51905 | 10: 55049581 | 51908 | 10: 55137727 | 51909 | 10: 55138450 |
| 51915 | 10: 55257378 | 51922 | 10: 55519496 | 51928 | 10: 55693450 | 51929 | 10: 55701600 | 51930 | 10: 55701734 |
| 51935 | 10: 55824628 | 51937 | 10: 55884610 | 51944 | 10: 56050425 | 51950 | 10: 56235551 | 51961 | 10: 56404358 |
| 51974 | 10: 56691062 | 52008 | 10: 57738832 | 52009 | 10: 57749028 | 52012 | 10: 57825646 | 52021 | 10: 58035128 |
| 52030 | 10: 58215720 | 52031 | 10: 58215742 | 52032 | 10: 58223900 | 52033 | 10: 58223933 | 52034 | 10: 58223992 |
| 52038 | 10: 58382992 | 52043 | 10: 58575796 | 52048 | 10: 58651910 | 52055 | 10: 58892988 | 52058 | 10: 58957454 |
| 52059 | 10: 58957455 | 52060 | 10: 58957462 | 52061 | 10: 58963341 | 52068 | 10: 59169515 | 52069 | 10: 59257696 |
| 52070 | 10: 59257779 | 52074 | 10: 59431442 | 52084 | 10: 59743414 | 52096 | 10: 60229145 | 52099 | 10: 60376158 |
| 52104 | 10: 60467179 | 52132 | 10: 61502794 | 52137 | 10: 61691415 | 52142 | 10: 61759883 | 52143 | 10: 61761276 |
| 52144 | 10: 61770176 | 52147 | 10: 61801949 | 52152 | 10: 61854226 | 52156 | 10: 61927985 | 52161 | 10: 62065140 |
| 52162 | 10: 62070975 | 52167 | 10: 62152633 | 52188 | 10: 62478919 | 52189 | 10: 62483660 | 52196 | 10: 62841361 |
| 52197 | 10: 62843919 | 52206 | 10: 63003189 | 52207 | 10: 63005018 | 52215 | 10: 63176255 | 52217 | 10: 63234235 |
| 52218 | 10: 63236141 | 52230 | 10: 63545031 | 52233 | 10: 63676002 | 52235 | 10: 63714642 | 52246 | 10: 64098165 |
| 52254 | 10: 64366724 | 52255 | 10: 64366769 | 52256 | 10: 64366784 | 52257 | 10: 64366788 | 52258 | 10: 64366808 |
| 52259 | 10: 64366813 | 52260 | 10: 64366843 | 52273 | 10: 64623835 | 52274 | 10: 64624712 | 52297 | 10: 65311772 |
| 52298 | 10: 65333523 | 52300 | 10: 65360982 | 52301 | 10: 65363558 | 52302 | 10: 65366448 | 52303 | 10: 65370682 |
| 52304 | 10: 65374137 | 52305 | 10: 65381281 | 52306 | 10: 65388071 | 52307 | 10: 65427744 | 52308 | 10: 65428255 |
| 52309 | 10: 65448723 | 52310 | 10: 65460822 | 52311 | 10: 65464926 | 52315 | 10: 65667285 | 52317 | 10: 65689392 |
| 52319 | 10: 65693892 | 52320 | 10: 65705467 | 52328 | 10: 65975774 | 52333 | 10: 66037974 | 52374 | 10: 66920995 |
| 52379 | 10: 67082677 | 52383 | 10: 67193245 | 52384 | 10: 67193602 | 52385 | 10: 67207585 | 52388 | 10: 67271539 |
| 52389 | 10: 67275755 | 52400 | 10: 67706373 | 52401 | 10: 67706382 | 52403 | 10: 67837002 | 52412 | 10: 67919519 |
| 52415 | 10: 68009703 | 52419 | 10: 68069027 | 52426 | 10: 68164248 | 52430 | 10: 68213913 | 52434 | 10: 68307915 |
| 52435 | 10: 68336932 | 52445 | 10: 68532314 | 52446 | 10: 68532338 | 52449 | 10: 68589455 | 52453 | 10: 68616316 |
| 52454 | 10: 68648835 | 52460 | 10: 68845801 | 52479 | 10: 69314335 | 52483 | 10: 69414869 | 52489 | 10: 69550619 |
| 52497 | 10: 69813170 | 52506 | 10: 69985799 | 52518 | 10: 70311136 | 52530 | 10: 70914151 | 52532 | 10: 70933695 |
| 52543 | 10: 71144557 | 52545 | 10: 71190058 | 52547 | 10: 71282860 | 52549 | 10: 71395114 | 52550 | 10: 71401946 |
| 52553 | 10: 71492246 | 52560 | 10: 71739567 | 52561 | 10: 71739616 | 52562 | 10: 71741589 | 52568 | 10: 71990029 |
| 52594 | 10: 72844951 | 52601 | 10: 73180235 | 52603 | 10: 73207644 | 52608 | 10: 73381610 | 52619 | 10: 73593430 |
| 52621 | 10: 73621273 | 52625 | 10: 73665667 | 52628 | 10: 73702186 | 52631 | 10: 73800772 | 52634 | 10: 73889706 |
| 52639 | 10: 74014694 | 52640 | 10: 74014740 | 52647 | 10: 74278788 | 52669 | 10: 75144155 | 52684 | 10: 75472993 |
| 52690 | 10: 75648302 | 52702 | 10: 76063641 | 52711 | 10: 76208279 | 52712 | 10: 76213571 | 52713 | 10: 76216092 |
| 52714 | 10: 76217503 | 52716 | 10: 76219150 | 52717 | 10: 76231514 | 52727 | 10: 76445843 | 52739 | 10: 76742309 |
| 52740 | 10: 76780386 | 52750 | 10: 76927979 | 52753 | 10: 77027256 | 52754 | 10: 77038645 | 52755 | 10: 77043267 |
| 52756 | 10: 77050549 | 52757 | 10: 77063990 | 52758 | 10: 77079521 | 52760 | 10: 77086192 | 52761 | 10: 77087944 |
| 52764 | 10: 77165808 | 52765 | 10: 77182957 | 52769 | 10: 77284463 | 52770 | 10: 77295759 | 52772 | 10: 77310958 |
| 52783 | 10: 77734390 | 52785 | 10: 77815166 | 52787 | 10: 77843400 | 52790 | 10: 77908553 | 52794 | 10: 77934474 |
| 52800 | 10: 78024784 | 52804 | 10: 78090692 | 52805 | 10: 78091383 | 52806 | 10: 78170657 | 52818 | 10: 78466873 |
| 52819 | 10: 78489026 | 52820 | 10: 78524325 | 52833 | 10: 78874750 | 52834 | 10: 78890533 | 52843 | 10: 79142128 |
| 52844 | 10: 79142222 | 52850 | 10: 79364696 | 52858 | 10: 79552918 | 52864 | 10: 79765450 | 52866 | 10: 79778751 |
| 52876 | 10: 80036481 | 52878 | 10: 80058483 | 52887 | 10: 80321841 | 52888 | 10: 80354807 | 52890 | 10: 80376119 |
| 52897 | 10: 80516438 | 52898 | 10: 80584353 | 52899 | 10: 80612799 | 52908 | 10: 80833008 | 52917 | 10: 81113106 |
| 52918 | 10: 81113113 | 52919 | 10: 81113154 | 52920 | 10: 81119799 | 52921 | 10: 81122203 | 52922 | 10: 81122225 |
| 52923 | 10: 81133087 | 52924 | 10: 81134221 | 52925 | 10: 81141810 | 52926 | 10: 81147906 | 52927 | 10: 81159861 |
| 52928 | 10: 81163746 | 52929 | 10: 81169707 | 52930 | 10: 81171404 | 52931 | 10: 81174589 | 52932 | 10: 81177743 |
| 52933 | 10: 81179444 | 52934 | 10: 81180179 | 52935 | 10: 81182426 | 52936 | 10: 81188153 | 52937 | 10: 81191624 |
| 52938 | 10: 81192985 | 52939 | 10: 81201260 | 52942 | 10: 81232104 | 52946 | 10: 81275274 | 52948 | 10: 81293569 |
| 52949 | 10: 81313921 | 52954 | 10: 81424405 | 52955 | 10: 81429276 | 52956 | 10: 81433859 | 52957 | 10: 81434033 |
| 52960 | 10: 81522739 | 52961 | 10: 81563853 | 52971 | 10: 81760421 | 52983 | 10: 81884426 | 52985 | 10: 81907059 |
| 52989 | 10: 82020430 | 52991 | 10: 82046798 | 52993 | 10: 82113076 | 52994 | 10: 82117432 | 52996 | 10: 82155665 |
| 53001 | 10: 82226992 | 53003 | 10: 82262769 | 53010 | 10: 82436819 | 53020 | 10: 82813082 | 53031 | 10: 83035771 |
| 53036 | 10: 83168495 | 53037 | 10: 83180678 | 53043 | 10: 83268162 | 53064 | 10: 83783323 | 53074 | 10: 83967539 |
| 53078 | 10: 84046423 | 53080 | 10: 84074336 | 53081 | 10: 84113189 | 53087 | 10: 84235120 | 53088 | 10: 84249302 |
| 53091 | 10: 84274673 | 53093 | 10: 84321770 | 53095 | 10: 84358684 | 53098 | 10: 84410074 | 53101 | 10: 84432430 |
| 53113 | 10: 84804439 | 53114 | 10: 84809599 | 53117 | 10: 84921371 | 53120 | 10: 84937878 | 53122 | 10: 84972916 |
| 53124 | 10: 85090540 | 53125 | 10: 85090775 | 53126 | 10: 85093417 | 53129 | 10: 85140109 | 53135 | 10: 85210872 |
| 53139 | 10: 85277645 | 53144 | 10: 85348496 | 53148 | 10: 85452360 | 53159 | 10: 85646771 | 53161 | 10: 85685359 |
| 53162 | 10: 85714779 | 53163 | 10: 85731082 | 53175 | 10: 86198306 | 53179 | 10: 86295239 | 53180 | 10: 86322591 |
| 53182 | 10: 86350641 | 53183 | 10: 86373524 | 53186 | 10: 86404676 | 53188 | 10: 86427269 | 53191 | 10: 86481134 |
| 53193 | 10: 86492591 | 53194 | 10: 86510757 | 53196 | 10: 86545729 | 53197 | 10: 86553401 | 53207 | 10: 86743362 |
| 53209 | 10: 86835796 | 53211 | 10: 86863386 | 53214 | 10: 86939644 | 53223 | 10: 87113458 | 53244 | 10: 87621927 |
| 53246 | 10: 87627438 | 53250 | 10: 87721184 | 53252 | 10: 87755864 | 53256 | 10: 87849396 | 53257 | 10: 87849463 |
| 53258 | 10: 87850909 | 53268 | 10: 88187681 | 53270 | 10: 88260182 | 53271 | 10: 88298292 | 53274 | 10: 88347395 |
| 53276 | 10: 88380317 | 53280 | 10: 88522805 | 53291 | 10: 88839037 | 53292 | 10: 88845164 | 53293 | 10: 88846191 |
| 53297 | 10: 88888537 | 53299 | 10: 88925587 | 53313 | 10: 89160492 | 53314 | 10: 89160521 | 53316 | 10: 89233565 |
| 53331 | 10: 89626724 | 53332 | 10: 89626819 | 53343 | 10: 89798617 | 53348 | 10: 89872058 | 53349 | 10: 89872136 |
| 53350 | 10: 89933077 | 53364 | 10: 90186331 | 53378 | 10: 90418700 | 53379 | 10: 90418707 | 53415 | 10: 90987301 |
| 53419 | 10: 91011354 | 53420 | 10: 91019729 | 53431 | 10: 91348121 | 53444 | 10: 91737619 | 53445 | 10: 91746178 |
| 53449 | 10: 91823694 | 53456 | 10: 92080780 | 53458 | 10: 92100804 | 53464 | 10: 92232897 | 53465 | 10: 92281587 |
| 53473 | 10: 92466754 | 53476 | 10: 92499113 | 53480 | 10: 92594602 | 53482 | 10: 92604984 | 53488 | 10: 92710477 |
| 53489 | 10: 92717162 | 53492 | 10: 92799252 | 53507 | 10: 93053048 | 53508 | 10: 93053064 | 53512 | 10: 93140659 |
| 53528 | 10: 93626548 | 53537 | 10: 93975527 | 53538 | 10: 93977121 | 53549 | 10: 94274383 | 53557 | 10: 94377341 |
| 53558 | 10: 94377386 | 53572 | 10: 94695544 | 53576 | 10: 94786454 | 53584 | 10: 95064914 | 53595 | 10: 95385183 |
| 53597 | 10: 95412469 | 53602 | 10: 95561991 | 53603 | 10: 95575347 | 53608 | 10: 95643374 | 53609 | 10: 95672545 |
| 53611 | 10: 95688501 | 53613 | 10: 95728606 | 53618 | 10: 95843138 | 53620 | 10: 95843176 | 53628 | 10: 96029745 |
| 53635 | 10: 96240911 | 53638 | 10: 96288699 | 53641 | 10: 96380513 | 53642 | 10: 96380599 | 53647 | 10: 96569247 |
| 53650 | 10: 96598589 | 53657 | 10: 96839484 | 53659 | 10: 96875821 | 53662 | 10: 96976508 | 53673 | 10: 97153819 |
| 53678 | 10: 97206516 | 53683 | 10: 97309948 | 53684 | 10: 97309954 | 53692 | 10: 97454671 | 53699 | 10: 97550303 |
| 53700 | 10: 97582963 | 53749 | 10: 98562531 | 53777 | 10: 99221264 | 53788 | 10: 99540676 | 53789 | 10: 99540722 |
| 53790 | 10: 99540732 | 53791 | 10: 99540816 | 53792 | 10: 99540849 | 53805 | 10: 99922832 | 53806 | 10: 99926816 |
| 53808 | 10: 99941485 | 53812 | 10: 10001067 | 53815 | 10: 10008239 | 53819 | 10: 10018045 | 53820 | 10: 10018777 |
| 53822 | 10: 10024360 | 53825 | 10: 10029136 | 53826 | 10: 10030147 | 53827 | 10: 10030338 | 53828 | 10: 10030391 |
| 53829 | 10: 10030447 | 53831 | 10: 10033080 | 53852 | 10: 10082168 | 53854 | 10: 10087653 | 53858 | 10: 10096073 |
| 53860 | 10: 10096399 | 53861 | 10: 10097431 | 53871 | 10: 10113021 | 53872 | 10: 10113562 | 53888 | 10: 10143538 |
| 53889 | 10: 10147495 | 53890 | 10: 10148861 | 53891 | 10: 10150021 | 53892 | 10: 10150656 | 53903 | 10: 10170837 |
| 53912 | 10: 10180575 | 53918 | 10: 10186280 | 53919 | 10: 10187041 | 53924 | 10: 10200983 | 53934 | 10: 10213583 |
| 53937 | 10: 10221227 | 53938 | 10: 10222090 | 53940 | 10: 10225283 | 53947 | 10: 10236108 | 53950 | 10: 10248171 |
| 53959 | 10: 10271339 | 53962 | 10: 10281920 | 54004 | 11: 441458 | 54008 | 11: 588104 | 54009 | 11: 612918 |
| 54010 | 11: 620469 | 54013 | 11: 675675 | 54015 | 11: 721258 | 54018 | 11: 743177 | 54023 | 11: 818693 |
| 54024 | 11: 818745 | 54025 | 11: 839682 | 54028 | 11: 884789 | 54029 | 11: 916613 | 54042 | 11: 1140277 |
| 54051 | 11: 1252661 | 54072 | 11: 1599964 | 54073 | 11: 1603522 | 54074 | 11: 1604088 | 54076 | 11: 1610503 |
| 54077 | 11: 1613029 | 54078 | 11: 1613069 | 54079 | 11: 1615691 | 54080 | 11: 1615825 | 54081 | 11: 1615860 |
| 54082 | 11: 1616045 | 54083 | 11: 1617470 | 54084 | 11: 1617491 | 54085 | 11: 1618013 | 54088 | 11: 1661775 |
| 54090 | 11: 1692551 | 54091 | 11: 1692581 | 54092 | 11: 1692586 | 54094 | 11: 1715577 | 54105 | 11: 1929102 |
| 54106 | 11: 1939516 | 54109 | 11: 1975548 | 54112 | 11: 2019047 | 54113 | 11: 2019051 | 54116 | 11: 2054152 |
| 54117 | 11: 2058128 | 54122 | 11: 2135119 | 54123 | 11: 2165271 | 54124 | 11: 2165552 | 54125 | 11: 2165653 |
| 54135 | 11: 2371904 | 54136 | 11: 2375767 | 54137 | 11: 2379791 | 54138 | 11: 2380701 | 54139 | 11: 2381359 |
| 54141 | 11: 2381820 | 54142 | 11: 2381938 | 54143 | 11: 2382261 | 54144 | 11: 2383322 | 54145 | 11: 2383432 |
| 54147 | 11: 2388794 | 54148 | 11: 2391923 | 54149 | 11: 2392633 | 54150 | 11: 2392687 | 54151 | 11: 2392744 |
| 54152 | 11: 2393551 | 54153 | 11: 2393601 | 54154 | 11: 2393614 | 54155 | 11: 2394563 | 54156 | 11: 2394565 |
| 54157 | 11: 2394754 | 54158 | 11: 2429820 | 54159 | 11: 2433742 | 54162 | 11: 2466683 | 54163 | 11: 2466765 |
| 54164 | 11: 2466767 | 54176 | 11: 2690324 | 54177 | 11: 2691733 | 54179 | 11: 2704089 | 54185 | 11: 2887185 |
| 54186 | 11: 2909443 | 54190 | 11: 2965363 | 54191 | 11: 2970198 | 54192 | 11: 2971406 | 54193 | 11: 2974009 |
| 54194 | 11: 3048024 | 54195 | 11: 3048066 | 54197 | 11: 3068135 | 54198 | 11: 3073771 | 54201 | 11: 3080087 |
| 54202 | 11: 3080140 | 54203 | 11: 3080321 | 54204 | 11: 3080329 | 54212 | 11: 3324474 | 54214 | 11: 3339436 |
| 54222 | 11: 3496384 | 54223 | 11: 3498026 | 54229 | 11: 3630171 | 54232 | 11: 3683284 | 54234 | 11: 3687500 |
| 54235 | 11: 3697811 | 54236 | 11: 3703164 | 54241 | 11: 3827658 | 54242 | 11: 3831757 | 54243 | 11: 3831764 |
| 54244 | 11: 3833403 | 54245 | 11: 3834470 | 54246 | 11: 3834544 | 54249 | 11: 3860402 | 54250 | 11: 3861478 |
| 54251 | 11: 3861495 | 54252 | 11: 3863319 | 54258 | 11: 4044668 | 54261 | 11: 4144540 | 54274 | 11: 4626193 |
| 54275 | 11: 4643628 | 54277 | 11: 4692004 | 54278 | 11: 4715218 | 54280 | 11: 4754025 | 54285 | 11: 4846290 |
| 54288 | 11: 4908167 | 54301 | 11: 5122618 | 54306 | 11: 5207458 | 54307 | 11: 5208484 | 54308 | 11: 5216864 |
| 54317 | 11: 5475369 | 54320 | 11: 5522837 | 54321 | 11: 5546069 | 54322 | 11: 5547645 | 54336 | 11: 5892298 |
| 54338 | 11: 5921461 | 54342 | 11: 6014407 | 54344 | 11: 6057385 | 54346 | 11: 6090351 | 54353 | 11: 6281176 |
| 54367 | 11: 6672389 | 54371 | 11: 6825527 | 54378 | 11: 7079505 | 54379 | 11: 7079550 | 54383 | 11: 7226247 |
| 54385 | 11: 7290568 | 54387 | 11: 7312202 | 54390 | 11: 7398056 | 54392 | 11: 7442481 | 54395 | 11: 7492967 |
| 54397 | 11: 7506651 | 54406 | 11: 7612707 | 54424 | 11: 8016040 | 54441 | 11: 8559109 | 54456 | 11: 9088843 |
| 54457 | 11: 9094665 | 54458 | 11: 9108482 | 54459 | 11: 9116906 | 54460 | 11: 9122303 | 54461 | 11: 9124299 |
| 54462 | 11: 9125436 | 54463 | 11: 9129918 | 54464 | 11: 9140181 | 54465 | 11: 9142056 | 54467 | 11: 9146703 |
| 54468 | 11: 9147988 | 54469 | 11: 9149728 | 54470 | 11: 9150188 | 54471 | 11: 9150797 | 54472 | 11: 9152234 |
| 54473 | 11: 9153632 | 54477 | 11: 9263561 | 54485 | 11: 9432577 | 54493 | 11: 9646962 | 54497 | 11: 9717817 |
| 54499 | 11: 9759593 | 54502 | 11: 9782467 | 54509 | 11: 9926241 | 54516 | 11: 10139900 | 54517 | 11: 10192082 |
| 54518 | 11: 10205888 | 54519 | 11: 10207241 | 54521 | 11: 10218640 | 54522 | 11: 10223170 | 54528 | 11: 10378672 |
| 54534 | 11: 10498811 | 54535 | 11: 10506565 | 54539 | 11: 10583951 | 54542 | 11: 10706952 | 54543 | 11: 10730447 |
| 54545 | 11: 10770592 | 54547 | 11: 10787415 | 54548 | 11: 10804935 | 54549 | 11: 10814646 | 54551 | 11: 10817795 |
| 54559 | 11: 11049635 | 54562 | 11: 11215341 | 54564 | 11: 11231984 | 54565 | 11: 11242289 | 54573 | 11: 11352441 |
| 54574 | 11: 11362173 | 54576 | 11: 11392077 | 54578 | 11: 11435064 | 54584 | 11: 11556298 | 54586 | 11: 11616272 |
| 54601 | 11: 12092660 | 54602 | 11: 12096347 | 54608 | 11: 12209379 | 54623 | 11: 12569024 | 54625 | 11: 12600200 |
| 54634 | 11: 12790767 | 54643 | 11: 13030163 | 54645 | 11: 13087614 | 54648 | 11: 13127507 | 54662 | 11: 13410435 |
| 54667 | 11: 13471629 | 54693 | 11: 14069498 | 54697 | 11: 14240512 | 54700 | 11: 14312963 | 54707 | 11: 14569196 |
| 54708 | 11: 14601805 | 54717 | 11: 14951793 | 54720 | 11: 15049901 | 54729 | 11: 15188618 | 54737 | 11: 15351949 |
| 54800 | 11: 16839124 | 54806 | 11: 17005906 | 54808 | 11: 17038164 | 54809 | 11: 17038230 | 54811 | 11: 17062733 |
| 54819 | 11: 17340238 | 54820 | 11: 17340240 | 54821 | 11: 17361542 | 54823 | 11: 17465587 | 54829 | 11: 17736027 |
| 54830 | 11: 17736142 | 54870 | 11: 18811490 | 54871 | 11: 18834617 | 54879 | 11: 19016954 | 54885 | 11: 19227167 |
| 54891 | 11: 19479022 | 54893 | 11: 19516116 | 54894 | 11: 19516281 | 54897 | 11: 19574521 | 54909 | 11: 20009995 |
| 54914 | 11: 20249271 | 54916 | 11: 20268750 | 54929 | 11: 20671302 | 54933 | 11: 20761364 | 54935 | 11: 20839310 |
| 54946 | 11: 21100669 | 54948 | 11: 21153847 | 54952 | 11: 21298331 | 54953 | 11: 21386222 | 54965 | 11: 21549331 |
| 54968 | 11: 21639708 | 54971 | 11: 21679170 | 54979 | 11: 21792131 | 54985 | 11: 21911853 | 54998 | 11: 22232061 |
| 54999 | 11: 22277590 | 55009 | 11: 22536318 | 55016 | 11: 22632697 | 55031 | 11: 23047670 | 55035 | 11: 23109116 |
| 55043 | 11: 23406318 | 55045 | 11: 23420733 | 55057 | 11: 23678147 | 55059 | 11: 23713701 | 55060 | 11: 23740579 |
| 55061 | 11: 23792727 | 55062 | 11: 23799914 | 55072 | 11: 23927783 | 55073 | 11: 23927808 | 55082 | 11: 24309077 |
| 55114 | 11: 24847692 | 55115 | 11: 24847723 | 55120 | 11: 25005279 | 55143 | 11: 25650622 | 55144 | 11: 25668988 |
| 55155 | 11: 26000399 | 55158 | 11: 26101828 | 55163 | 11: 26155816 | 55171 | 11: 26293504 | 55172 | 11: 26294033 |
| 55175 | 11: 26405127 | 55176 | 11: 26406745 | 55179 | 11: 26441367 | 55184 | 11: 26597807 | 55202 | 11: 27150645 |
| 55210 | 11: 27309958 | 55223 | 11: 27583040 | 55224 | 11: 27612153 | 55225 | 11: 27648326 | 55226 | 11: 27664648 |
| 55227 | 11: 27673338 | 55235 | 11: 27863723 | 55243 | 11: 28050164 | 55244 | 11: 28054041 | 55254 | 11: 28269292 |
| 55255 | 11: 28312159 | 55256 | 11: 28314792 | 55259 | 11: 28347672 | 55261 | 11: 28406003 | 55270 | 11: 28648699 |
| 55271 | 11: 28677976 | 55272 | 11: 28697612 | 55275 | 11: 28750175 | 55288 | 11: 28998567 | 55292 | 11: 29029776 |
| 55294 | 11: 29094181 | 55305 | 11: 29333853 | 55306 | 11: 29339273 | 55311 | 11: 29433875 | 55312 | 11: 29446589 |
| 55314 | 11: 29483268 | 55316 | 11: 29523821 | 55320 | 11: 29644262 | 55321 | 11: 29652000 | 55328 | 11: 29750755 |
| 55338 | 11: 29944937 | 55339 | 11: 30022269 | 55347 | 11: 30283476 | 55357 | 11: 30480608 | 55358 | 11: 30487514 |
| 55359 | 11: 30490953 | 55360 | 11: 30498423 | 55363 | 11: 30695691 | 55371 | 11: 30816430 | 55375 | 11: 30852665 |
| 55378 | 11: 30909842 | 55381 | 11: 31003265 | 55386 | 11: 31096059 | 55394 | 11: 31338792 | 55395 | 11: 31338818 |
| 55396 | 11: 31338832 | 55418 | 11: 31989218 | 55419 | 11: 32020262 | 55427 | 11: 32309370 | 55440 | 11: 32659947 |
| 55451 | 11: 33038916 | 55454 | 11: 33061320 | 55455 | 11: 33063315 | 55465 | 11: 33230608 | 55473 | 11: 33483917 |
| 55497 | 11: 34161881 | 55512 | 11: 34992407 | 55515 | 11: 35028247 | 55518 | 11: 35081009 | 55523 | 11: 35271551 |
| 55550 | 11: 35973444 | 55555 | 11: 36103640 | 55567 | 11: 36415088 | 55575 | 11: 36677962 | 55578 | 11: 36729555 |
| 55579 | 11: 36730226 | 55584 | 11: 36935209 | 55590 | 11: 37042393 | 55592 | 11: 37065356 | 55593 | 11: 37084548 |
| 55597 | 11: 37151685 | 55602 | 11: 37273838 | 55607 | 11: 37321094 | 55610 | 11: 37357247 | 55617 | 11: 37555217 |
| 55622 | 11: 37622807 | 55623 | 11: 37622964 | 55629 | 11: 37686699 | 55631 | 11: 37699061 | 55632 | 11: 37708915 |
| 55639 | 11: 37993013 | 55640 | 11: 38023147 | 55644 | 11: 38167213 | 55652 | 11: 38524637 | 55676 | 11: 39146746 |
| 55698 | 11: 39953399 | 55719 | 11: 40780484 | 55720 | 11: 40781022 | 55725 | 11: 40977806 | 55729 | 11: 41081305 |
| 55731 | 11: 41095065 | 55732 | 11: 41108333 | 55733 | 11: 41112618 | 55734 | 11: 41119005 | 55735 | 11: 41119970 |
| 55737 | 11: 41122253 | 55738 | 11: 41130588 | 55739 | 11: 41131422 | 55740 | 11: 41138457 | 55742 | 11: 41163401 |
| 55743 | 11: 41165333 | 55744 | 11: 41172319 | 55745 | 11: 41190502 | 55746 | 11: 41211212 | 55747 | 11: 41236765 |
| 55757 | 11: 41353565 | 55785 | 11: 42015177 | 55813 | 11: 42568364 | 55819 | 11: 42700906 | 55838 | 11: 43239984 |
| 55862 | 11: 43925979 | 55864 | 11: 44029565 | 55869 | 11: 44191991 | 55875 | 11: 44335233 | 55877 | 11: 44398012 |
| 55884 | 11: 44616476 | 55885 | 11: 44669598 | 55886 | 11: 44720675 | 55891 | 11: 44864177 | 55892 | 11: 44872149 |
| 55895 | 11: 44907130 | 55896 | 11: 44910831 | 55897 | 11: 44915340 | 55899 | 11: 44947747 | 55900 | 11: 44960989 |
| 55901 | 11: 44979580 | 55905 | 11: 45085243 | 55910 | 11: 45233285 | 55912 | 11: 45240869 | 55926 | 11: 45875224 |
| 55930 | 11: 45909750 | 55932 | 11: 45965043 | 55935 | 11: 46005981 | 55939 | 11: 46042064 | 55944 | 11: 46118783 |
| 55948 | 11: 46189212 | 55966 | 11: 46586822 | 55984 | 11: 46788369 | 55985 | 11: 46796356 | 55986 | 11: 46801200 |
| 55990 | 11: 46887994 | 55991 | 11: 46893010 | 55992 | 11: 46895379 | 55993 | 11: 46895384 | 55994 | 11: 46895386 |
| 55995 | 11: 46895443 | 55996 | 11: 46896988 | 55997 | 11: 46914387 | 55998 | 11: 46915926 | 56001 | 11: 46933431 |
| 56002 | 11: 46946293 | 56009 | 11: 47133868 | 56010 | 11: 47136307 | 56012 | 11: 47153161 | 56016 | 11: 47318477 |
| 56020 | 11: 47346660 | 56023 | 11: 47402450 | 56025 | 11: 47421701 | 56028 | 11: 47484468 | 56039 | 11: 47868107 |
| 56041 | 11: 47957513 | 56051 | 11: 48153310 | 56052 | 11: 48179620 | 56055 | 11: 48232533 | 56057 | 11: 48284436 |
| 56064 | 11: 48495828 | 56065 | 11: 48519034 | 56066 | 11: 48519745 | 56077 | 11: 48624594 | 56078 | 11: 48641618 |
| 56079 | 11: 48643769 | 56080 | 11: 48648683 | 56081 | 11: 48652079 | 56082 | 11: 48653016 | 56083 | 11: 48653703 |
| 56084 | 11: 48659221 | 56086 | 11: 48661468 | 56087 | 11: 48662095 | 56088 | 11: 48663888 | 56089 | 11: 48664547 |
| 56090 | 11: 48665440 | 56091 | 11: 48666103 | 56092 | 11: 48666658 | 56093 | 11: 48673397 | 56094 | 11: 48673919 |
| 56095 | 11: 48674533 | 56096 | 11: 48675652 | 56097 | 11: 48676148 | 56098 | 11: 48677162 | 56099 | 11: 48679806 |
| 56100 | 11: 48680593 | 56101 | 11: 48682804 | 56102 | 11: 48685811 | 56103 | 11: 48690618 | 56104 | 11: 48695696 |
| 56105 | 11: 48703362 | 56107 | 11: 48712410 | 56108 | 11: 48715777 | 56109 | 11: 48729623 | 56110 | 11: 48732364 |
| 56111 | 11: 48740101 | 56112 | 11: 48740643 | 56113 | 11: 48742231 | 56114 | 11: 48745355 | 56115 | 11: 48748086 |
| 56116 | 11: 48757634 | 56117 | 11: 48788737 | 56118 | 11: 48792299 | 56120 | 11: 48910719 | 56134 | 11: 49167307 |
| 56142 | 11: 49394511 | 56144 | 11: 49406127 | 56146 | 11: 49419581 | 56151 | 11: 49574160 | 56163 | 11: 49832758 |
| 56164 | 11: 49852553 | 56165 | 11: 49852648 | 56200 | 11: 51131191 | 56203 | 11: 51184125 | 56220 | 11: 51745809 |
| 56243 | 11: 52697360 | 56265 | 11: 53468115 | 56274 | 11: 53833063 | 56290 | 11: 54327571 | 56298 | 11: 54566473 |
| 56300 | 11: 54590112 | 56320 | 11: 54776336 | 56323 | 11: 54807232 | 56328 | 11: 54930465 | 56329 | 11: 54961590 |
| 56340 | 11: 55186748 | 56347 | 11: 55369267 | 56355 | 11: 55552815 | 56356 | 11: 55571320 | 56363 | 11: 55683959 |
| 56364 | 11: 55696252 | 56378 | 11: 56190217 | 56402 | 11: 56970533 | 56428 | 11: 57877493 | 56439 | 11: 58140156 |
| 56493 | 11: 59622606 | 56497 | 11: 59747111 | 56500 | 11: 59861239 | 56510 | 11: 60271069 | 56511 | 11: 60271074 |
| 56512 | 11: 60271117 | 56513 | 11: 60271193 | 56520 | 11: 60465745 | 56521 | 11: 60465915 | 56522 | 11: 60465920 |
| 56523 | 11: 60465923 | 56524 | 11: 60468952 | 56525 | 11: 60470373 | 56526 | 11: 60470388 | 56527 | 11: 60471668 |
| 56529 | 11: 60472081 | 56530 | 11: 60474103 | 56531 | 11: 60474134 | 56532 | 11: 60474983 | 56533 | 11: 60475508 |
| 56534 | 11: 60475664 | 56535 | 11: 60478021 | 56536 | 11: 60478028 | 56537 | 11: 60478049 | 56538 | 11: 60478204 |
| 56540 | 11: 60494769 | 56541 | 11: 60494791 | 56542 | 11: 60517676 | 56543 | 11: 60566671 | 56544 | 11: 60567347 |
| 56545 | 11: 60567387 | 56546 | 11: 60616886 | 56549 | 11: 60641170 | 56550 | 11: 60641182 | 56553 | 11: 60671076 |
| 56557 | 11: 60758092 | 56558 | 11: 60775322 | 56560 | 11: 60906972 | 56562 | 11: 60949119 | 56564 | 11: 60961745 |
| 56566 | 11: 61011277 | 56567 | 11: 61011512 | 56570 | 11: 61079196 | 56573 | 11: 61139067 | 56574 | 11: 61149300 |
| 56575 | 11: 61149340 | 56576 | 11: 61149341 | 56578 | 11: 61149398 | 56579 | 11: 61149409 | 56581 | 11: 61172809 |
| 56585 | 11: 61361997 | 56588 | 11: 61539166 | 56590 | 11: 61548155 | 56606 | 11: 62153933 | 56616 | 11: 62410952 |
| 56621 | 11: 62549111 | 56627 | 11: 62639185 | 56629 | 11: 62703391 | 56637 | 11: 62848946 | 56645 | 11: 63079035 |
| 56647 | 11: 63132879 | 56657 | 11: 63363010 | 56658 | 11: 63376645 | 56661 | 11: 63394494 | 56665 | 11: 63456586 |
| 56671 | 11: 63645957 | 56672 | 11: 63664952 | 56676 | 11: 63733057 | 56679 | 11: 63799850 | 56687 | 11: 63862724 |
| 56691 | 11: 63954830 | 56693 | 11: 63980633 | 56694 | 11: 63980697 | 56695 | 11: 64007710 | 56708 | 11: 64345353 |
| 56712 | 11: 64472503 | 56716 | 11: 64576376 | 56718 | 11: 64684536 | 56721 | 11: 64807816 | 56726 | 11: 64972478 |
| 56735 | 11: 65170349 | 56736 | 11: 65170393 | 56741 | 11: 65341010 | 56743 | 11: 65372142 | 56746 | 11: 65438305 |
| 56766 | 11: 65998851 | 56769 | 11: 66070592 | 56775 | 11: 66159407 | 56782 | 11: 66307327 | 56783 | 11: 66315381 |
| 56790 | 11: 66473480 | 56795 | 11: 66556801 | 56796 | 11: 66556834 | 56799 | 11: 66629962 | 56813 | 11: 67084838 |
| 56814 | 11: 67108786 | 56815 | 11: 67114080 | 56821 | 11: 67213238 | 56829 | 11: 67439320 | 56832 | 11: 67453087 |
| 56834 | 11: 67479748 | 56835 | 11: 67481439 | 56836 | 11: 67483125 | 56838 | 11: 67512057 | 56842 | 11: 67544307 |
| 56843 | 11: 67549232 | 56844 | 11: 67549363 | 56846 | 11: 67556514 | 56847 | 11: 67563313 | 56848 | 11: 67582936 |
| 56849 | 11: 67582999 | 56850 | 11: 67583014 | 56852 | 11: 67588734 | 56855 | 11: 67702445 | 56856 | 11: 67709590 |
| 56857 | 11: 67727467 | 56858 | 11: 67728102 | 56859 | 11: 67730113 | 56860 | 11: 67736102 | 56861 | 11: 67737073 |
| 56862 | 11: 67747241 | 56863 | 11: 67749685 | 56864 | 11: 67752237 | 56865 | 11: 67759745 | 56867 | 11: 67762854 |
| 56868 | 11: 67767430 | 56869 | 11: 67772466 | 56870 | 11: 67776969 | 56871 | 11: 67787768 | 56872 | 11: 67799383 |
| 56873 | 11: 67805662 | 56874 | 11: 67810598 | 56879 | 11: 67896985 | 56882 | 11: 67970890 | 56885 | 11: 68111905 |
| 56893 | 11: 68411391 | 56897 | 11: 68467750 | 56909 | 11: 68787390 | 56921 | 11: 69071509 | 56932 | 11: 69177619 |
| 56939 | 11: 69288577 | 56941 | 11: 69302814 | 56947 | 11: 69479974 | 56950 | 11: 69514045 | 56953 | 11: 69570750 |
| 56954 | 11: 69572243 | 56956 | 11: 69619243 | 56957 | 11: 69620020 | 56958 | 11: 69620110 | 56960 | 11: 69662827 |
| 56967 | 11: 69745012 | 56992 | 11: 70292588 | 56994 | 11: 70341851 | 56995 | 11: 70350355 | 57003 | 11: 70595154 |
| 57010 | 11: 70714155 | 57011 | 11: 70718274 | 57013 | 11: 70755284 | 57014 | 11: 70761156 | 57015 | 11: 70761179 |
| 57016 | 11: 70761872 | 57017 | 11: 70762170 | 57018 | 11: 70762689 | 57019 | 11: 70762700 | 57020 | 11: 70762702 |
| 57021 | 11: 70762781 | 57022 | 11: 70762862 | 57023 | 11: 70762868 | 57024 | 11: 70763022 | 57025 | 11: 70763076 |
| 57026 | 11: 70763145 | 57027 | 11: 70763553 | 57028 | 11: 70763715 | 57029 | 11: 70763906 | 57030 | 11: 70764185 |
| 57031 | 11: 70764539 | 57032 | 11: 70768163 | 57033 | 11: 70768281 | 57034 | 11: 70769360 | 57035 | 11: 70769405 |
| 57036 | 11: 70775819 | 57037 | 11: 70782767 | 57038 | 11: 70782825 | 57039 | 11: 70806849 | 57040 | 11: 70807701 |
| 57041 | 11: 70810781 | 57043 | 11: 70862638 | 57048 | 11: 71062901 | 57049 | 11: 71062934 | 57053 | 11: 71130818 |
| 57054 | 11: 71131623 | 57057 | 11: 71142342 | 57059 | 11: 71149248 | 57066 | 11: 71175312 | 57067 | 11: 71177865 |
| 57068 | 11: 71178203 | 57070 | 11: 71182683 | 57072 | 11: 71198214 | 57073 | 11: 71199013 | 57074 | 11: 71208751 |
| 57081 | 11: 71398446 | 57094 | 11: 71666080 | 57095 | 11: 71713947 | 57099 | 11: 71827640 | 57102 | 11: 71961107 |
| 57103 | 11: 71974375 | 57108 | 11: 72101578 | 57112 | 11: 72181248 | 57128 | 11: 72810370 | 57133 | 11: 72851670 |
| 57144 | 11: 72988812 | 57146 | 11: 73025557 | 57148 | 11: 73068186 | 57150 | 11: 73079672 | 57151 | 11: 73079767 |
| 57152 | 11: 73084466 | 57153 | 11: 73087461 | 57154 | 11: 73089558 | 57155 | 11: 73090095 | 57158 | 11: 73133312 |
| 57159 | 11: 73148896 | 57161 | 11: 73219332 | 57163 | 11: 73277111 | 57166 | 11: 73438358 | 57167 | 11: 73480475 |
| 57177 | 11: 73781192 | 57181 | 11: 73853326 | 57185 | 11: 73967619 | 57188 | 11: 74009513 | 57190 | 11: 74018063 |
| 57194 | 11: 74054528 | 57205 | 11: 74195149 | 57206 | 11: 74204674 | 57207 | 11: 74205573 | 57209 | 11: 74245610 |
| 57213 | 11: 74399369 | 57218 | 11: 74544795 | 57227 | 11: 74974483 | 57230 | 11: 75017890 | 57232 | 11: 75044406 |
| 57235 | 11: 75106218 | 57236 | 11: 75198521 | 57242 | 11: 75328149 | 57243 | 11: 75330425 | 57245 | 11: 75334844 |
| 57246 | 11: 75384323 | 57249 | 11: 75420509 | 57250 | 11: 75420581 | 57251 | 11: 75477129 | 57252 | 11: 75482256 |
| 57254 | 11: 75493333 | 57256 | 11: 75509869 | 57257 | 11: 75513821 | 57266 | 11: 75686867 | 57273 | 11: 75929380 |
| 57280 | 11: 76017351 | 57309 | 11: 76876371 | 57310 | 11: 76952531 | 57321 | 11: 77091873 | 57322 | 11: 77092007 |
| 57323 | 11: 77121176 | 57324 | 11: 77123614 | 57325 | 11: 77124254 | 57327 | 11: 77132776 | 57332 | 11: 77262392 |
| 57355 | 11: 77817486 | 57362 | 11: 77896861 | 57363 | 11: 77897300 | 57364 | 11: 77897480 | 57365 | 11: 77897481 |
| 57366 | 11: 77897524 | 57367 | 11: 77899208 | 57368 | 11: 77899796 | 57369 | 11: 77900345 | 57370 | 11: 77900437 |
| 57371 | 11: 77900488 | 57372 | 11: 77900522 | 57373 | 11: 77901876 | 57374 | 11: 77901997 | 57375 | 11: 77916472 |
| 57376 | 11: 77916653 | 57377 | 11: 77917793 | 57378 | 11: 77918799 | 57379 | 11: 77920101 | 57380 | 11: 77920618 |
| 57381 | 11: 77920740 | 57382 | 11: 77920832 | 57384 | 11: 77922179 | 57385 | 11: 77922588 | 57386 | 11: 77923067 |
| 57387 | 11: 77927350 | 57398 | 11: 78226756 | 57401 | 11: 78268974 | 57402 | 11: 78275050 | 57403 | 11: 78303645 |
| 57407 | 11: 78375724 | 57412 | 11: 78529857 | 57415 | 11: 78631628 | 57417 | 11: 78663074 | 57419 | 11: 78724728 |
| 57430 | 11: 78997006 | 57438 | 11: 79084041 | 57452 | 11: 79229476 | 57454 | 11: 79233862 | 57479 | 11: 79764049 |
| 57489 | 11: 80152318 | 57494 | 11: 80309473 | 57496 | 11: 80321806 | 57505 | 11: 80474151 | 57507 | 11: 80513212 |
| 57514 | 11: 80648310 | 57515 | 11: 80671943 | 57516 | 11: 80690342 | 57545 | 11: 81671201 | 57546 | 11: 81671227 |
| 57551 | 11: 81785970 | 57552 | 11: 81800692 | 57558 | 11: 81881809 | 57575 | 11: 82260811 | 57580 | 11: 82351733 |
| 57583 | 11: 82364482 | 57591 | 11: 82664554 | 57592 | 11: 82664600 | 57593 | 11: 82664609 | 57594 | 11: 82664725 |
| 57596 | 11: 82671761 | 57597 | 11: 82677475 | 57598 | 11: 82677728 | 57599 | 11: 82683785 | 57600 | 11: 82686842 |
| 57601 | 11: 82687013 | 57602 | 11: 82687072 | 57603 | 11: 82687666 | 57604 | 11: 82700336 | 57605 | 11: 82709413 |
| 57621 | 11: 83006401 | 57622 | 11: 83027036 | 57623 | 11: 83038638 | 57629 | 11: 83118274 | 57630 | 11: 83137062 |
| 57652 | 11: 83676352 | 57692 | 11: 84965273 | 57709 | 11: 85278395 | 57711 | 11: 85316120 | 57712 | 11: 85350978 |
| 57720 | 11: 85498225 | 57722 | 11: 85595081 | 57731 | 11: 85746062 | 57733 | 11: 85771451 | 57736 | 11: 85853743 |
| 57738 | 11: 85936758 | 57748 | 11: 86094641 | 57750 | 11: 86113594 | 57753 | 11: 86201573 | 57757 | 11: 86221979 |
| 57758 | 11: 86224683 | 57789 | 11: 87198869 | 57790 | 11: 87208683 | 57792 | 11: 87231125 | 57797 | 11: 87357375 |
| 57798 | 11: 87380616 | 57803 | 11: 87492469 | 57804 | 11: 87494268 | 57805 | 11: 87495906 | 57807 | 11: 87520617 |
| 57808 | 11: 87530939 | 57812 | 11: 87650800 | 57815 | 11: 87710317 | 57821 | 11: 87813732 | 57823 | 11: 87882552 |
| 57829 | 11: 87970096 | 57830 | 11: 87972654 | 57831 | 11: 87972763 | 57832 | 11: 87975372 | 57833 | 11: 87980700 |
| 57834 | 11: 87982062 | 57835 | 11: 87982864 | 57836 | 11: 87986573 | 57837 | 11: 87987392 | 57838 | 11: 87991576 |
| 57839 | 11: 87992353 | 57840 | 11: 87994077 | 57841 | 11: 87994761 | 57842 | 11: 87995976 | 57843 | 11: 87996439 |
| 57844 | 11: 87997041 | 57845 | 11: 87997518 | 57846 | 11: 87998024 | 57848 | 11: 88000459 | 57849 | 11: 88001097 |
| 57850 | 11: 88002138 | 57851 | 11: 88003290 | 57852 | 11: 88006051 | 57853 | 11: 88006580 | 57854 | 11: 88007139 |
| 57855 | 11: 88008201 | 57857 | 11: 88009081 | 57858 | 11: 88009883 | 57859 | 11: 88011349 | 57860 | 11: 88013047 |
| 57862 | 11: 88020282 | 57863 | 11: 88021880 | 57864 | 11: 88030641 | 57866 | 11: 88045066 | 57873 | 11: 88105264 |
| 57885 | 11: 88479108 | 57890 | 11: 88733341 | 57897 | 11: 88922785 | 57898 | 11: 88922827 | 57899 | 11: 88922860 |
| 57902 | 11: 88976021 | 57904 | 11: 88994635 | 57909 | 11: 89132552 | 57910 | 11: 89133642 | 57911 | 11: 89152187 |
| 57912 | 11: 89213759 | 57919 | 11: 89375784 | 57920 | 11: 89387726 | 57929 | 11: 89561970 | 57940 | 11: 89843767 |
| 57960 | 11: 90408075 | 57965 | 11: 90443507 | 57970 | 11: 90503652 | 57977 | 11: 90744044 | 57979 | 11: 90785269 |
| 57980 | 11: 90787434 | 57984 | 11: 90906061 | 57989 | 11: 90982703 | 57991 | 11: 91054916 | 57994 | 11: 91084013 |
| 58002 | 11: 91266370 | 58006 | 11: 91343800 | 58061 | 11: 92402829 | 58062 | 11: 92454234 | 58063 | 11: 92473780 |
| 58064 | 11: 92476182 | 58073 | 11: 92661797 | 58074 | 11: 92674766 | 58077 | 11: 92715331 | 58084 | 11: 92833095 |
| 58095 | 11: 93210615 | 58098 | 11: 93223406 | 58116 | 11: 93629686 | 58119 | 11: 93727304 | 58126 | 11: 93972515 |
| 58127 | 11: 93973132 | 58128 | 11: 93973249 | 58129 | 11: 93973432 | 58131 | 11: 94067508 | 58132 | 11: 94076855 |
| 58137 | 11: 94178079 | 58140 | 11: 94211102 | 58144 | 11: 94339459 | 58148 | 11: 94453289 | 58151 | 11: 94500564 |
| 58152 | 11: 94531181 | 58164 | 11: 94880948 | 58173 | 11: 95280882 | 58177 | 11: 95315895 | 58178 | 11: 95322263 |
| 58181 | 11: 95425616 | 58190 | 11: 95586651 | 58193 | 11: 95692063 | 58201 | 11: 95840093 | 58207 | 11: 96117932 |
| 58209 | 11: 96142116 | 58213 | 11: 96261764 | 58215 | 11: 96285921 | 58216 | 11: 96322123 | 58217 | 11: 96322228 |
| 58218 | 11: 96338458 | 58230 | 11: 96820150 | 58245 | 11: 97196967 | 58248 | 11: 97225356 | 58251 | 11: 97254785 |
| 58253 | 11: 97286893 | 58276 | 11: 97843712 | 58277 | 11: 97843818 | 58278 | 11: 97883599 | 58279 | 11: 97892319 |
| 58289 | 11: 98162222 | 58290 | 11: 98162477 | 58291 | 11: 98173675 | 58292 | 11: 98179996 | 58293 | 11: 98189444 |
| 58294 | 11: 98191816 | 58295 | 11: 98193416 | 58298 | 11: 98222190 | 58304 | 11: 98396073 | 58313 | 11: 98726635 |
| 58316 | 11: 98813314 | 58321 | 11: 98866430 | 58327 | 11: 98921444 | 58328 | 11: 98921481 | 58331 | 11: 98929180 |
| 58332 | 11: 98929534 | 58333 | 11: 98931991 | 58334 | 11: 98932587 | 58335 | 11: 98951509 | 58337 | 11: 98980770 |
| 58338 | 11: 99034896 | 58339 | 11: 99114771 | 58342 | 11: 99223633 | 58343 | 11: 99225289 | 58346 | 11: 99292200 |
| 58347 | 11: 99366886 | 58348 | 11: 99391843 | 58349 | 11: 99395118 | 58350 | 11: 99395609 | 58351 | 11: 99396169 |
| 58352 | 11: 99397143 | 58353 | 11: 99398436 | 58354 | 11: 99399218 | 58355 | 11: 99400709 | 58356 | 11: 99401571 |
| 58357 | 11: 99402294 | 58358 | 11: 99403416 | 58359 | 11: 99409144 | 58360 | 11: 99424233 | 58361 | 11: 99434036 |
| 58362 | 11: 99444575 | 58368 | 11: 99668885 | 58378 | 11: 99888002 | 58382 | 11: 99923257 | 58383 | 11: 99932292 |
| 58384 | 11: 99932294 | 58392 | 11: 10002865 | 58402 | 11: 10020523 | 58404 | 11: 10023875 | 58405 | 11: 10024462 |
| 58408 | 11: 10034694 | 58413 | 11: 10046250 | 58421 | 11: 10065868 | 58423 | 11: 10069663 | 58432 | 11: 10089413 |
| 58433 | 11: 10089414 | 58434 | 11: 10089418 | 58435 | 11: 10089420 | 58436 | 11: 10089422 | 58437 | 11: 10089423 |
| 58438 | 11: 10089584 | 58439 | 11: 10089862 | 58440 | 11: 10089910 | 58441 | 11: 10090030 | 58442 | 11: 10090083 |
| 58443 | 11: 10090139 | 58444 | 11: 10090298 | 58445 | 11: 10090301 | 58446 | 11: 10090303 | 58447 | 11: 10090310 |
| 58448 | 11: 10090437 | 58449 | 11: 10090548 | 58450 | 11: 10090552 | 58451 | 11: 10090559 | 58452 | 11: 10092003 |
| 58453 | 11: 10092018 | 58455 | 11: 10092948 | 58456 | 11: 10092991 | 58465 | 11: 10121113 | 58469 | 11: 10139576 |
| 58481 | 11: 10176766 | 58482 | 11: 10179134 | 58490 | 11: 10195025 | 58498 | 11: 10216529 | 58503 | 11: 10235669 |
| 58505 | 11: 10236915 | 58507 | 11: 10245112 | 58508 | 11: 10245553 | 58509 | 11: 10245561 | 58510 | 11: 10245564 |
| 58512 | 11: 10248040 | 58520 | 11: 10260314 | 58521 | 11: 10260318 | 58527 | 11: 10273417 | 58534 | 11: 10280891 |
| 58535 | 11: 10282294 | 58537 | 11: 10284704 | 58539 | 11: 10287469 | 58540 | 11: 10287825 | 58541 | 11: 10287894 |
| 58542 | 11: 10288318 | 58543 | 11: 10288773 | 58544 | 11: 10289321 | 58545 | 11: 10289432 | 58546 | 11: 10289512 |
| 58547 | 11: 10289609 | 58548 | 11: 10289667 | 58549 | 11: 10289886 | 58550 | 11: 10289974 | 58551 | 11: 10290070 |
| 58552 | 11: 10290097 | 58553 | 11: 10290321 | 58555 | 11: 10293724 | 58561 | 11: 10303129 | 58563 | 11: 10311374 |
| 58565 | 11: 10312784 | 58570 | 11: 10325911 | 58571 | 11: 10325913 | 58572 | 11: 10325914 | 58573 | 11: 10325923 |
| 58574 | 11: 10327300 | 58580 | 11: 10337533 | 58582 | 11: 10338027 | 58586 | 11: 10342689 | 58599 | 11: 10376543 |
| 58603 | 11: 10389419 | 58610 | 11: 10416326 | 58611 | 11: 10416332 | 58614 | 11: 10418835 | 58616 | 11: 10420735 |
| 58621 | 11: 10424706 | 58626 | 11: 10440574 | 58641 | 11: 10466512 | 58647 | 11: 10471723 | 58648 | 11: 10472240 |
| 58664 | 11: 10499756 | 58666 | 11: 10508228 | 58669 | 11: 10514921 | 58675 | 11: 10524663 | 58676 | 11: 10526769 |
| 58678 | 11: 10536852 | 58679 | 11: 10536862 | 58682 | 11: 10549667 | 58683 | 11: 10556546 | 58684 | 11: 10556548 |
| 58685 | 11: 10556557 | 58686 | 11: 10558647 | 58687 | 11: 10559874 | 58688 | 11: 10560239 | 58689 | 11: 10560299 |
| 58690 | 11: 10560406 | 58691 | 11: 10560652 | 58692 | 11: 10560775 | 58693 | 11: 10560879 | 58694 | 11: 10560986 |
| 58695 | 11: 10561194 | 58696 | 11: 10561355 | 58697 | 11: 10561585 | 58698 | 11: 10561748 | 58699 | 11: 10561828 |
| 58700 | 11: 10562085 | 58701 | 11: 10562158 | 58702 | 11: 10562413 | 58703 | 11: 10562929 | 58704 | 11: 10567074 |
| 58705 | 11: 10569241 | 58707 | 11: 10576176 | 58712 | 11: 10606865 | 58713 | 11: 10608376 | 58715 | 11: 10621054 |
| 58717 | 11: 10634575 | 58719 | 11: 10644949 | 58722 | 11: 10657444 | 58723 | 11: 10657459 | 58724 | 11: 10657485 |
| 58725 | 11: 10661179 | 58726 | 11: 10661476 | 58727 | 11: 10662633 | 58740 | 12: 317570 | 58748 | 12: 542288 |
| 58757 | 12: 877215 | 58797 | 12: 1923479 | 58805 | 12: 2038461 | 58813 | 12: 2256922 | 58819 | 12: 2323582 |
| 58825 | 12: 2448377 | 58826 | 12: 2448435 | 58827 | 12: 2452533 | 58828 | 12: 2452685 | 58829 | 12: 2509817 |
| 58836 | 12: 2603168 | 58839 | 12: 2644727 | 58840 | 12: 2644772 | 58841 | 12: 2644793 | 58844 | 12: 2731342 |
| 58846 | 12: 2771593 | 58923 | 12: 4734740 | 58937 | 12: 5055337 | 59006 | 12: 7477665 | 59040 | 12: 8789258 |
| 59041 | 12: 8796877 | 59053 | 12: 9132925 | 59062 | 12: 9382289 | 59066 | 12: 9448144 | 59077 | 12: 9708609 |
| 59078 | 12: 9708626 | 59093 | 12: 10135415 | 59095 | 12: 10160043 | 59114 | 12: 10462955 | 59122 | 12: 10621365 |
| 59123 | 12: 10622131 | 59124 | 12: 10631694 | 59125 | 12: 10631700 | 59134 | 12: 10781199 | 59152 | 12: 11193032 |
| 59170 | 12: 11622983 | 59171 | 12: 11641143 | 59173 | 12: 11670644 | 59175 | 12: 11684828 | 59186 | 12: 11878651 |
| 59189 | 12: 11933919 | 59193 | 12: 12026687 | 59195 | 12: 12066263 | 59196 | 12: 12069687 | 59197 | 12: 12087862 |
| 59198 | 12: 12123346 | 59206 | 12: 12322089 | 59210 | 12: 12404824 | 59219 | 12: 12760273 | 59224 | 12: 12974557 |
| 59231 | 12: 13161754 | 59239 | 12: 13388932 | 59241 | 12: 13411406 | 59242 | 12: 13473520 | 59243 | 12: 13518818 |
| 59246 | 12: 13578439 | 59247 | 12: 13591408 | 59250 | 12: 13656437 | 59261 | 12: 13824057 | 59268 | 12: 13927173 |
| 59280 | 12: 14131140 | 59281 | 12: 14132377 | 59283 | 12: 14157283 | 59285 | 12: 14211580 | 59286 | 12: 14211590 |
| 59287 | 12: 14211610 | 59288 | 12: 14240924 | 59292 | 12: 14361824 | 59293 | 12: 14372218 | 59294 | 12: 14388145 |
| 59295 | 12: 14399933 | 59299 | 12: 14456910 | 59302 | 12: 14520853 | 59306 | 12: 14595181 | 59311 | 12: 14716329 |
| 59319 | 12: 14988572 | 59321 | 12: 15005389 | 59324 | 12: 15101552 | 59333 | 12: 15266724 | 59335 | 12: 15281976 |
| 59344 | 12: 15465428 | 59348 | 12: 15534714 | 59350 | 12: 15552667 | 59352 | 12: 15567714 | 59353 | 12: 15577383 |
| 59355 | 12: 15618825 | 59360 | 12: 15729746 | 59363 | 12: 15770061 | 59366 | 12: 15810335 | 59373 | 12: 15938324 |
| 59380 | 12: 16125597 | 59381 | 12: 16136554 | 59386 | 12: 16274543 | 59390 | 12: 16346416 | 59394 | 12: 16413245 |
| 59410 | 12: 16793613 | 59414 | 12: 16900417 | 59423 | 12: 17162528 | 59425 | 12: 17199573 | 59430 | 12: 17324217 |
| 59435 | 12: 17368614 | 59440 | 12: 17470036 | 59453 | 12: 17926477 | 59459 | 12: 18040185 | 59468 | 12: 18229565 |
| 59469 | 12: 18253619 | 59474 | 12: 18390032 | 59475 | 12: 18391314 | 59486 | 12: 18697557 | 59492 | 12: 18898137 |
| 59496 | 12: 18919039 | 59507 | 12: 19071459 | 59509 | 12: 19100832 | 59517 | 12: 19186475 | 59519 | 12: 19221055 |
| 59520 | 12: 19233240 | 59522 | 12: 19351299 | 59523 | 12: 19367974 | 59524 | 12: 19367977 | 59528 | 12: 19460445 |
| 59545 | 12: 19931195 | 59550 | 12: 20111148 | 59551 | 12: 20111169 | 59578 | 12: 20706397 | 59582 | 12: 20914485 |
| 59588 | 12: 21042616 | 59591 | 12: 21070776 | 59597 | 12: 21212930 | 59600 | 12: 21309091 | 59602 | 12: 21368915 |
| 59613 | 12: 21811288 | 59617 | 12: 21924603 | 59618 | 12: 21936752 | 59620 | 12: 21945571 | 59621 | 12: 21969243 |
| 59627 | 12: 22146118 | 59631 | 12: 22263714 | 59642 | 12: 22573355 | 59643 | 12: 22576349 | 59647 | 12: 22662928 |
| 59653 | 12: 22821830 | 59654 | 12: 22834458 | 59663 | 12: 23060365 | 59667 | 12: 23201662 | 59671 | 12: 23249052 |
| 59677 | 12: 23371031 | 59680 | 12: 23439101 | 59696 | 12: 23931995 | 59703 | 12: 24076421 | 59710 | 12: 24256758 |
| 59716 | 12: 24331867 | 59721 | 12: 24471307 | 59733 | 12: 24731505 | 59734 | 12: 24748284 | 59737 | 12: 24838866 |
| 59741 | 12: 24922215 | 59742 | 12: 24925957 | 59747 | 12: 25022781 | 59749 | 12: 25034947 | 59754 | 12: 25118490 |
| 59766 | 12: 25470724 | 59767 | 12: 25498467 | 59769 | 12: 25589790 | 59777 | 12: 25724323 | 59798 | 12: 26442722 |
| 59805 | 12: 26653552 | 59808 | 12: 26751983 | 59814 | 12: 26874891 | 59820 | 12: 26979789 | 59821 | 12: 26986008 |
| 59830 | 12: 27189356 | 59841 | 12: 27380634 | 59848 | 12: 27503259 | 59850 | 12: 27528591 | 59854 | 12: 27721180 |
| 59855 | 12: 27723132 | 59856 | 12: 27723229 | 59861 | 12: 27998192 | 59862 | 12: 28002030 | 59878 | 12: 28526148 |
| 59879 | 12: 28587383 | 59881 | 12: 28621183 | 59893 | 12: 28989343 | 59913 | 12: 29539899 | 59934 | 12: 30141838 |
| 59944 | 12: 30381579 | 59945 | 12: 30411312 | 59947 | 12: 30444618 | 59949 | 12: 30463422 | 59969 | 12: 30909596 |
| 59971 | 12: 30979325 | 59973 | 12: 30983862 | 59974 | 12: 30991739 | 59977 | 12: 31053899 | 59979 | 12: 31059835 |
| 59980 | 12: 31115741 | 59984 | 12: 31247300 | 59985 | 12: 31247421 | 59986 | 12: 31247454 | 59987 | 12: 31247486 |
| 59998 | 12: 31454833 | 60007 | 12: 31767190 | 60011 | 12: 31790778 | 60012 | 12: 31797116 | 60018 | 12: 31950273 |
| 60023 | 12: 32162560 | 60028 | 12: 32295933 | 60029 | 12: 32338936 | 60032 | 12: 32379592 | 60033 | 12: 32387590 |
| 60034 | 12: 32391405 | 60037 | 12: 32450230 | 60038 | 12: 32450232 | 60040 | 12: 32487563 | 60058 | 12: 33054434 |
| 60061 | 12: 33082346 | 60062 | 12: 33085573 | 60066 | 12: 33139330 | 60075 | 12: 33340233 | 60086 | 12: 33674224 |
| 60095 | 12: 33970418 | 60096 | 12: 34054782 | 60097 | 12: 34068458 | 60109 | 12: 34500254 | 60114 | 12: 34681917 |
| 60126 | 12: 34900938 | 60133 | 12: 35115665 | 60140 | 12: 35310855 | 60150 | 12: 35346851 | 60164 | 12: 35747252 |
| 60173 | 12: 36216803 | 60175 | 12: 36341507 | 60176 | 12: 36361684 | 60178 | 12: 36421378 | 60179 | 12: 36443014 |
| 60181 | 12: 36474506 | 60182 | 12: 36483162 | 60183 | 12: 36486137 | 60184 | 12: 36504213 | 60189 | 12: 36706435 |
| 60191 | 12: 36753159 | 60195 | 12: 36952382 | 60196 | 12: 36952416 | 60210 | 12: 37329971 | 60211 | 12: 37330062 |
| 60212 | 12: 37330099 | 60216 | 12: 37420551 | 60256 | 12: 39391630 | 60260 | 12: 39553608 | 60267 | 12: 39684127 |
| 60270 | 12: 39776454 | 60279 | 12: 40049870 | 60281 | 12: 40141911 | 60311 | 12: 41076939 | 60329 | 12: 41861946 |
| 60332 | 12: 41912763 | 60402 | 12: 44937336 | 60404 | 12: 44979318 | 60422 | 12: 45617161 | 60442 | 12: 46329415 |
| 60445 | 12: 46430991 | 60466 | 12: 46961986 | 60470 | 12: 47018801 | 60474 | 12: 47110627 | 60484 | 12: 47309976 |
| 60487 | 12: 47407515 | 60508 | 12: 48047961 | 60510 | 12: 48150915 | 60538 | 12: 48960299 | 60549 | 12: 49199679 |
| 60552 | 12: 49236831 | 60566 | 12: 49561422 | 60571 | 12: 49723456 | 60574 | 12: 49789492 | 60579 | 12: 49873319 |
| 60580 | 12: 49881472 | 60584 | 12: 49919596 | 60600 | 12: 50189626 | 60609 | 12: 50384404 | 60611 | 12: 50424103 |
| 60612 | 12: 50439814 | 60614 | 12: 50497707 | 60615 | 12: 50517145 | 60616 | 12: 50525554 | 60617 | 12: 50530876 |
| 60619 | 12: 50579721 | 60628 | 12: 50806532 | 60630 | 12: 50840958 | 60632 | 12: 50869818 | 60633 | 12: 50871829 |
| 60660 | 12: 51551817 | 60661 | 12: 51626384 | 60679 | 12: 52103992 | 60687 | 12: 52262646 | 60692 | 12: 52346386 |
| 60702 | 12: 52660295 | 60710 | 12: 52806268 | 60727 | 12: 53053258 | 60729 | 12: 53061165 | 60738 | 12: 53338165 |
| 60739 | 12: 53339046 | 60740 | 12: 53339054 | 60749 | 12: 53523285 | 60750 | 12: 53553256 | 60752 | 12: 53587669 |
| 60755 | 12: 53627763 | 60756 | 12: 53640047 | 60763 | 12: 53801258 | 60781 | 12: 54131348 | 60782 | 12: 54153871 |
| 60797 | 12: 54494995 | 60803 | 12: 54630943 | 60804 | 12: 54634286 | 60810 | 12: 54736016 | 60813 | 12: 54833257 |
| 60815 | 12: 54927343 | 60819 | 12: 55024877 | 60821 | 12: 55068443 | 60848 | 12: 55943119 | 60851 | 12: 56039366 |
| 60880 | 12: 56976868 | 60882 | 12: 57050432 | 60904 | 12: 57798387 | 60905 | 12: 57798425 | 60912 | 12: 57952881 |
| 60914 | 12: 58028126 | 60929 | 12: 58672694 | 60957 | 12: 59417372 | 60969 | 12: 59853749 | 60978 | 12: 60236147 |
| 60990 | 12: 60736444 | 61018 | 12: 61855777 | 61024 | 12: 62071314 | 61028 | 12: 62095959 | 61031 | 12: 62215048 |
| 61034 | 12: 62291744 | 61035 | 12: 62297558 | 61051 | 12: 62665927 | 61053 | 12: 62697756 | 61065 | 12: 63053238 |
| 61080 | 12: 63589109 | 61085 | 12: 63730818 | 61089 | 12: 63774437 | 61094 | 12: 63920843 | 61100 | 12: 64086485 |
| 61112 | 12: 64454937 | 61126 | 12: 64874897 | 61127 | 12: 64896393 | 61148 | 12: 65376279 | 61151 | 12: 65433100 |
| 61156 | 12: 65616303 | 61168 | 12: 66123917 | 61170 | 12: 66204351 | 61175 | 12: 66340744 | 61199 | 12: 66896772 |
| 61206 | 12: 67184352 | 61207 | 12: 67228835 | 61210 | 12: 67289617 | 61216 | 12: 67417050 | 61231 | 12: 67757020 |
| 61237 | 12: 67889485 | 61252 | 12: 68144427 | 61255 | 12: 68171444 | 61256 | 12: 68171491 | 61262 | 12: 68316664 |
| 61274 | 12: 68605490 | 61280 | 12: 68760089 | 61283 | 12: 68784416 | 61289 | 12: 68861830 | 61321 | 12: 69512279 |
| 61322 | 12: 69512294 | 61323 | 12: 69516800 | 61331 | 12: 69577020 | 61338 | 12: 69681798 | 61348 | 12: 70147062 |
| 61367 | 12: 71314524 | 61383 | 12: 71639973 | 61386 | 12: 71790421 | 61390 | 12: 71842435 | 61408 | 12: 72931638 |
| 61414 | 12: 73012734 | 61421 | 12: 73132371 | 61422 | 12: 73154971 | 61427 | 12: 73317927 | 61428 | 12: 73324264 |
| 61441 | 12: 73504167 | 61443 | 12: 73515714 | 61444 | 12: 73515717 | 61445 | 12: 73515812 | 61454 | 12: 73622472 |
| 61455 | 12: 73637550 | 61462 | 12: 73750586 | 61469 | 12: 73899680 | 61475 | 12: 74054821 | 61486 | 12: 74328198 |
| 61495 | 12: 74492364 | 61498 | 12: 74505038 | 61500 | 12: 74526665 | 61517 | 12: 74949213 | 61530 | 12: 75243236 |
| 61535 | 12: 75318404 | 61538 | 12: 75375959 | 61542 | 12: 75403639 | 61543 | 12: 75471804 | 61558 | 12: 75839630 |
| 61567 | 12: 76074190 | 61569 | 12: 76110122 | 61581 | 12: 76477335 | 61587 | 12: 76644169 | 61588 | 12: 76649683 |
| 61605 | 12: 77139238 | 61609 | 12: 77240177 | 61627 | 12: 77604409 | 61632 | 12: 77695108 | 61641 | 12: 77891817 |
| 61644 | 12: 77992115 | 61647 | 12: 78041886 | 61655 | 12: 78246588 | 61669 | 12: 78577349 | 61691 | 12: 79095439 |
| 61692 | 12: 79098661 | 61693 | 12: 79098791 | 61694 | 12: 79099207 | 61695 | 12: 79099246 | 61696 | 12: 79104872 |
| 61710 | 12: 79403358 | 61715 | 12: 79476337 | 61727 | 12: 79736085 | 61740 | 12: 79970178 | 61756 | 12: 80319360 |
| 61775 | 12: 80804698 | 61790 | 12: 81214960 | 61791 | 12: 81215027 | 61803 | 12: 81507963 | 61804 | 12: 81508005 |
| 61819 | 12: 81781296 | 61820 | 12: 81840070 | 61824 | 12: 81902349 | 61840 | 12: 82263825 | 61845 | 12: 82445861 |
| 61848 | 12: 82510342 | 61877 | 12: 83071777 | 61882 | 12: 83242885 | 61891 | 12: 83362092 | 61907 | 12: 83699595 |
| 61915 | 12: 83925298 | 61920 | 12: 84006533 | 61926 | 12: 84137136 | 61945 | 12: 84691330 | 61946 | 12: 84709160 |
| 61948 | 12: 84744945 | 61949 | 12: 84744950 | 61955 | 12: 84883821 | 61960 | 12: 84929565 | 61966 | 12: 84993056 |
| 61969 | 12: 85038979 | 61970 | 12: 85051267 | 61973 | 12: 85122781 | 61978 | 12: 85226982 | 61979 | 12: 85230036 |
| 61981 | 12: 85245683 | 61983 | 12: 85268459 | 61984 | 12: 85293670 | 61986 | 12: 85321955 | 61988 | 12: 85328690 |
| 61990 | 12: 85352315 | 61991 | 12: 85354082 | 61994 | 12: 85362756 | 61995 | 12: 85375388 | 61997 | 12: 85396298 |
| 62000 | 12: 85462507 | 62007 | 12: 85632578 | 62013 | 12: 85790348 | 62014 | 12: 85790349 | 62035 | 12: 86313847 |
| 62038 | 12: 86341413 | 62043 | 12: 86507763 | 62045 | 12: 86516372 | 62046 | 12: 86516421 | 62048 | 12: 86519761 |
| 62049 | 12: 86565634 | 52051 | 12: 86656646 | 62052 | 12: 86687289 | 62053 | 12: 86688052 | 62056 | 12: 86734179 |
| 62058 | 12: 86744155 | 62059 | 12: 86764063 | 62062 | 12: 86796745 | 62064 | 12: 86824616 | 62068 | 12: 86895391 |
| 62069 | 12: 86895562 | 62070 | 12: 86897090 | 62071 | 12: 86897178 | 62072 | 12: 86901814 | 62073 | 12: 86901834 |
| 62074 | 12: 86901846 | 62075 | 12: 86901945 | 62076 | 12: 86902939 | 62077 | 12: 86902946 | 62078 | 12: 86907527 |
| 62079 | 12: 86907549 | 62080 | 12: 86908054 | 62082 | 12: 86924401 | 62085 | 12: 86976449 | 62086 | 12: 86988484 |
| 62089 | 12: 87019731 | 62090 | 12: 87024506 | 62095 | 12: 87094553 | 62109 | 13: 837989 | 62110 | 13: 859954 |
| 62115 | 13: 877219 | 62122 | 13: 1025761 | 62126 | 13: 1174223 | 62127 | 13: 1184941 | 62138 | 13: 1435091 |
| 62143 | 13: 1589719 | 62145 | 13: 1615667 | 62154 | 13: 1741335 | 62155 | 13: 1754933 | 62165 | 13: 2014401 |
| 62169 | 13: 2079335 | 62174 | 13: 2108318 | 62176 | 13: 2133989 | 62181 | 13: 2232512 | 62188 | 13: 2272732 |
| 62193 | 13: 2391867 | 62194 | 13: 2394109 | 62195 | 13: 2400920 | 62196 | 13: 2402378 | 62200 | 13: 2507817 |
| 62203 | 13: 2551762 | 62208 | 13: 2625076 | 62209 | 13: 2632788 | 62238 | 13: 3279851 | 62250 | 13: 3588425 |
| 62254 | 13: 3701619 | 62259 | 13: 3860445 | 62265 | 13: 3974443 | 62270 | 13: 4084139 | 62271 | 13: 4084171 |
| 62272 | 13: 4084181 | 62276 | 13: 4221428 | 62278 | 13: 4302438 | 62291 | 13: 4792202 | 62292 | 13: 4793338 |
| 62312 | 13: 5324308 | 62314 | 13: 5360261 | 62325 | 13: 5525194 | 62326 | 13: 5525225 | 62327 | 13: 5525250 |
| 62341 | 13: 5742073 | 62356 | 13: 6112364 | 62377 | 13: 6686807 | 62379 | 13: 6694933 | 62384 | 13: 6837817 |
| 62392 | 13: 7036389 | 62399 | 13: 7224958 | 62406 | 13: 7441639 | 62412 | 13: 7553513 | 62425 | 13: 7848557 |
| 62441 | 13: 8487756 | 62465 | 13: 8980654 | 62477 | 13: 9357735 | 62486 | 13: 9550333 | 62487 | 13: 9576478 |
| 62496 | 13: 9729764 | 62497 | 13: 9729772 | 62512 | 13: 10026414 | 62514 | 13: 10070381 | 62522 | 13: 10306871 |
| 62523 | 13: 10395756 | 62529 | 13: 10478767 | 62533 | 13: 10517565 | 62542 | 13: 10720404 | 62543 | 13: 10724228 |
| 62547 | 13: 10815809 | 62549 | 13: 10956279 | 62550 | 13: 10956346 | 62551 | 13: 10956354 | 62552 | 13: 10956355 |
| 62559 | 13: 11536433 | 62561 | 13: 11561251 | 62562 | 13: 11598409 | 62566 | 13: 11792199 | 62568 | 13: 11904569 |
| 62581 | 13: 12520500 | 62584 | 13: 12616676 | 62585 | 13: 12669714 | 62593 | 13: 12825549 | 62597 | 13: 12901657 |
| 62601 | 13: 12957260 | 62604 | 13: 13014675 | 62606 | 13: 13050532 | 62609 | 13: 13134686 | 62620 | 13: 13394218 |
| 62624 | 13: 13416442 | 62629 | 13: 13525172 | 62633 | 13: 13724404 | 62648 | 13: 14180541 | 62709 | 13: 15922152 |
| 62710 | 13: 15926948 | 62713 | 13: 16113457 | 62714 | 13: 16141551 | 62715 | 13: 16161619 | 62716 | 13: 16198035 |
| 62727 | 13: 16479721 | 62752 | 13: 16821257 | 62753 | 13: 16838893 | 62758 | 13: 16979210 | 62766 | 13: 17176766 |
| 62775 | 13: 17311025 | 62776 | 13: 17311026 | 62777 | 13: 17311048 | 62778 | 13: 17311402 | 62779 | 13: 17313556 |
| 62780 | 13: 17321384 | 62781 | 13: 17327212 | 62784 | 13: 17371815 | 62791 | 13: 17532109 | 62792 | 13: 17537709 |
| 62793 | 13: 17537777 | 62794 | 13: 17537795 | 62795 | 13: 17540260 | 62796 | 13: 17540346 | 62797 | 13: 17543085 |
| 62800 | 13: 17568204 | 62804 | 13: 17707891 | 62805 | 13: 17739721 | 62807 | 13: 17780066 | 62809 | 13: 17793717 |
| 62810 | 13: 17796108 | 62811 | 13: 17797006 | 62812 | 13: 17797028 | 62813 | 13: 17797078 | 62814 | 13: 17800892 |
| 62819 | 13: 17959881 | 62821 | 13: 18000186 | 62822 | 13: 18000196 | 62823 | 13: 18001014 | 62824 | 13: 18001496 |
| 62825 | 13: 18001505 | 62826 | 13: 18001601 | 62828 | 13: 18117994 | 62831 | 13: 18160335 | 62843 | 13: 18626070 |
| 62847 | 13: 18666175 | 62853 | 13: 18721208 | 62881 | 13: 18755905 | 62883 | 13: 18792716 | 62884 | 13: 18792825 |
| 62885 | 13: 18792850 | 62889 | 13: 18828811 | 62891 | 13: 18892498 | 62892 | 13: 18897253 | 62894 | 13: 19005851 |
| 62895 | 13: 19009052 | 62896 | 13: 19009093 | 62897 | 13: 19009113 | 62902 | 13: 19106619 | 62911 | 13: 19331702 |
| 62917 | 13: 19457766 | 62919 | 13: 19497053 | 62920 | 13: 19497075 | 62921 | 13: 19497132 | 62922 | 13: 19497137 |
| 62923 | 13: 19539755 | 62924 | 13: 19567930 | 62926 | 13: 19588565 | 62929 | 13: 19655690 | 62930 | 13: 19688739 |
| 62933 | 13: 19726581 | 62935 | 13: 19733380 | 62941 | 13: 19822361 | 62942 | 13: 19829540 | 62948 | 13: 19908550 |
| 62950 | 13: 19929408 | 62951 | 13: 19985595 | 62966 | 13: 20376052 | 62973 | 13: 20455853 | 62974 | 13: 20455858 |
| 62975 | 13: 20455892 | 62976 | 13: 20455934 | 62977 | 13: 20455941 | 62986 | 13: 20690765 | 62989 | 13: 20711502 |
| 63003 | 13: 21079068 | 63004 | 13: 21080622 | 63005 | 13: 21080632 | 63006 | 13: 21080639 | 63007 | 13: 21080653 |
| 63008 | 13: 21080676 | 63009 | 13: 21080695 | 63015 | 13: 21196496 | 63021 | 13: 21338503 | 63025 | 13: 21463530 |
| 63033 | 13: 21602178 | 63034 | 13: 21608876 | 63036 | 13: 21617194 | 63037 | 13: 21669388 | 63038 | 13: 21673325 |
| 63046 | 13: 21871770 | 63053 | 13: 22005224 | 63067 | 13: 22364650 | 63069 | 13: 22400352 | 63075 | 13: 22556435 |
| 63083 | 13: 22843425 | 63085 | 13: 22943752 | 63094 | 13: 23364320 | 63095 | 13: 23428241 | 63100 | 13: 23590170 |
| 63107 | 13: 23799970 | 63113 | 13: 23993051 | 63122 | 13: 24204881 | 63125 | 13: 24378395 | 63128 | 13: 24432283 |
| 63129 | 13: 24464855 | 63138 | 13: 24550804 | 63144 | 13: 24721448 | 63148 | 13: 24785943 | 63152 | 13: 24889747 |
| 63157 | 13: 25026767 | 63162 | 13: 25139101 | 63163 | 13: 25157343 | 63164 | 13: 25169602 | 63171 | 13: 25314039 |
| 63176 | 13: 25373782 | 63177 | 13: 25408450 | 63178 | 13: 25410196 | 63185 | 13: 25553780 | 63186 | 13: 25556386 |
| 63197 | 13: 25892715 | 63200 | 13: 25911303 | 63201 | 13: 25911339 | 63202 | 13: 25911366 | 63204 | 13: 25958875 |
| 63207 | 13: 26100997 | 63216 | 13: 26381171 | 63217 | 13: 26385197 | 63218 | 13: 26403532 | 63228 | 13: 26709761 |
| 63229 | 13: 26798911 | 63246 | 13: 27211452 | 63249 | 13: 27252426 | 63255 | 13: 27371552 | 63259 | 13: 27465175 |
| 63260 | 13: 27465215 | 63261 | 13: 27465241 | 63262 | 13: 27465291 | 63264 | 13: 27517101 | 63267 | 13: 27585269 |
| 63269 | 13: 27634615 | 63274 | 13: 27760115 | 63275 | 13: 27831742 | 63276 | 13: 27837915 | 63277 | 13: 27868901 |
| 63278 | 13: 27871914 | 63279 | 13: 27897181 | 63280 | 13: 27902374 | 63281 | 13: 27904185 | 63293 | 13: 28285297 |
| 63298 | 13: 28416787 | 63306 | 13: 28593403 | 63307 | 13: 28631602 | 63309 | 13: 28668818 | 63317 | 13: 28768035 |
| 63322 | 13: 28950265 | 63330 | 13: 29182643 | 63337 | 13: 29330098 | 63339 | 13: 29336139 | 63350 | 13: 29535530 |
| 63353 | 13: 29588319 | 63357 | 13: 29751352 | 63369 | 13: 30024809 | 63371 | 13: 30056042 | 63375 | 13: 30104514 |
| 63376 | 13: 30183328 | 63377 | 13: 30183348 | 63378 | 13: 30194908 | 63379 | 13: 30194914 | 63380 | 13: 30194921 |
| 63381 | 13: 30194933 | 63382 | 13: 30195026 | 63383 | 13: 30195496 | 63385 | 13: 30210577 | 63388 | 13: 30242235 |
| 63408 | 13: 30707478 | 63410 | 13: 30754086 | 63418 | 13: 30976341 | 63422 | 13: 31020731 | 63437 | 13: 31276749 |
| 63438 | 13: 31276801 | 63440 | 13: 31276837 | 63441 | 13: 31280242 | 63442 | 13: 31280491 | 63443 | 13: 31282636 |
| 63444 | 13: 31282742 | 63445 | 13: 31282787 | 63446 | 13: 31285967 | 63447 | 13: 31285968 | 63448 | 13: 31285972 |
| 63449 | 13: 31286001 | 63450 | 13: 31286035 | 63451 | 13: 31296423 | 63456 | 13: 31420376 | 63459 | 13: 31447543 |
| 63466 | 13: 31582342 | 63471 | 13: 31736295 | 63488 | 13: 32202239 | 63491 | 13: 32240332 | 63496 | 13: 32324178 |
| 63497 | 13: 32362115 | 63499 | 13: 32365033 | 63527 | 13: 32808897 | 63538 | 13: 33049577 | 63540 | 13: 33078146 |
| 63545 | 13: 33129981 | 63547 | 13: 33172919 | 63549 | 13: 33261503 | 63554 | 13: 33326303 | 63561 | 13: 33610056 |
| 63562 | 13: 33610145 | 63563 | 13: 33615532 | 63564 | 13: 33641897 | 63566 | 13: 33682852 | 63569 | 13: 33817581 |
| 63570 | 13: 33818040 | 63573 | 13: 33900260 | 63584 | 13: 34538815 | 63586 | 13: 34570424 | 63587 | 13: 34574425 |
| 63588 | 13: 34595352 | 63592 | 13: 34690679 | 63594 | 13: 34725177 | 63596 | 13: 34746582 | 63597 | 13: 34750823 |
| 63598 | 13: 34798730 | 63603 | 13: 34909612 | 63607 | 13: 35010608 | 63608 | 13: 35019310 | 63610 | 13: 35093002 |
| 63613 | 13: 35138794 | 63616 | 13: 35156141 | 63620 | 13: 35193780 | 63624 | 13: 35229937 | 63626 | 13: 35230259 |
| 63629 | 13: 35259809 | 63630 | 13: 35273048 | 63632 | 13: 35319711 | 63674 | 13: 36409030 | 63675 | 13: 36424590 |
| 63680 | 13: 36570662 | 63682 | 13: 36589718 | 63687 | 13: 36656277 | 63691 | 13: 36716029 | 63694 | 13: 36827015 |
| 63696 | 13: 36854156 | 63720 | 13: 37443281 | 63732 | 13: 37745409 | 63742 | 13: 37991091 | 63757 | 13: 38329758 |
| 63758 | 13: 38375463 | 63760 | 13: 38420421 | 63762 | 13: 38501340 | 63776 | 13: 38828149 | 63777 | 13: 38828176 |
| 63778 | 13: 38828210 | 63779 | 13: 38828306 | 63780 | 13: 38828333 | 63784 | 13: 38935644 | 63787 | 13: 38957716 |
| 63806 | 13: 39270018 | 63808 | 13: 39276440 | 63813 | 13: 39359409 | 63814 | 13: 39360537 | 63817 | 13: 39403535 |
| 63821 | 13: 39464756 | 63822 | 13: 39498069 | 63824 | 13: 39559729 | 63825 | 13: 39574123 | 63826 | 13: 39584391 |
| 63828 | 13: 39644803 | 63834 | 13: 39704818 | 63847 | 13: 40027121 | 63901 | 13: 41141472 | 63926 | 13: 41826984 |
| 63928 | 13: 41862833 | 63929 | 13: 41863502 | 63944 | 13: 42197339 | 63952 | 13: 42439893 | 63956 | 13: 42526261 |
| 63961 | 13: 42557806 | 63963 | 13: 42567447 | 63964 | 13: 42570926 | 63965 | 13: 42576337 | 63966 | 13: 42578756 |
| 63967 | 13: 42579747 | 63968 | 13: 42581051 | 63969 | 13: 42583033 | 63970 | 13: 42585954 | 63971 | 13: 42587350 |
| 63973 | 13: 42588548 | 63974 | 13: 42589734 | 63975 | 13: 42590562 | 63976 | 13: 42591034 | 63977 | 13: 42591591 |
| 63978 | 13: 42592688 | 63979 | 13: 42602259 | 63980 | 13: 42604226 | 63981 | 13: 42605060 | 63982 | 13: 42606861 |
| 63983 | 13: 42607539 | 63984 | 13: 42617225 | 63985 | 13: 42619772 | 63986 | 13: 42620639 | 63987 | 13: 42622791 |
| 63988 | 13: 42624718 | 63990 | 13: 42628160 | 63991 | 13: 42632412 | 63992 | 13: 42636254 | 63994 | 13: 42655005 |
| 63995 | 13: 42655747 | 63997 | 13: 42707046 | 63998 | 13: 42733395 | 64000 | 13: 42775404 | 64002 | 13: 42816216 |
| 64010 | 13: 43060399 | 64013 | 13: 43174367 | 64017 | 13: 43269543 | 64018 | 13: 43342598 | 64019 | 13: 43400200 |
| 64021 | 13: 43503495 | 64030 | 13: 43617243 | 64034 | 13: 43735619 | 64035 | 13: 43736152 | 64061 | 13: 44136436 |
| 64064 | 13: 44159827 | 64066 | 13: 44231058 | 64070 | 13: 44319398 | 64073 | 13: 44364216 | 64079 | 13: 44475918 |
| 64080 | 13: 44491717 | 64091 | 13: 44736057 | 64116 | 13: 44978580 | 64117 | 13: 44978611 | 64119 | 13: 44989622 |
| 64128 | 13: 45018113 | 64129 | 13: 45022424 | 64130 | 13: 45034606 | 64132 | 13: 45038863 | 64133 | 13: 45046560 |
| 64135 | 13: 45057233 | 64136 | 13: 45081132 | 64138 | 13: 45099582 | 64139 | 13: 45108541 | 64140 | 13: 45110718 |
| 64141 | 13: 45112674 | 64142 | 13: 45114484 | 64144 | 13: 45129342 | 64145 | 13: 45135756 | 64146 | 13: 45143400 |
| 64147 | 13: 45144409 | 64149 | 13: 45160765 | 64173 | 13: 45837065 | 64188 | 13: 46248201 | 64189 | 13: 46260736 |
| 64190 | 13: 46262101 | 64191 | 13: 46280773 | 64192 | 13: 46303988 | 64193 | 13: 46308862 | 64197 | 13: 46342659 |
| 64203 | 13: 46433525 | 64205 | 13: 46541983 | 64217 | 13: 46759585 | 64219 | 13: 46767424 | 64222 | 13: 46855501 |
| 64226 | 13: 46934942 | 64230 | 13: 47057469 | 64235 | 13: 47187424 | 64240 | 13: 47305456 | 64242 | 13: 47333121 |
| 64243 | 13: 47346135 | 64265 | 13: 47860032 | 64267 | 13: 47916526 | 64268 | 13: 47927079 | 64270 | 13: 47952062 |
| 64271 | 13: 48003258 | 64272 | 13: 48017728 | 64279 | 13: 48210178 | 64281 | 13: 48212858 | 64313 | 13: 48933264 |
| 64327 | 13: 49369652 | 64371 | 13: 50683553 | 64377 | 13: 50955038 | 64379 | 13: 50985036 | 64385 | 13: 51191490 |
| 64388 | 13: 51268713 | 64389 | 13: 51269069 | 64404 | 13: 51645253 | 64408 | 13: 51745594 | 64412 | 13: 51889992 |
| 64413 | 13: 51917960 | 64424 | 13: 52280855 | 64425 | 13: 52333810 | 64430 | 13: 52514427 | 64436 | 13: 52666460 |
| 64443 | 13: 52794690 | 64444 | 13: 52797973 | 64445 | 13: 52798735 | 64446 | 13: 52798786 | 64448 | 13: 52806170 |
| 64450 | 13: 52838262 | 64457 | 13: 53001236 | 64461 | 13: 53139865 | 64463 | 13: 53146985 | 64465 | 13: 53149275 |
| 64468 | 13: 53184275 | 64470 | 13: 53189308 | 64471 | 13: 53191327 | 64472 | 13: 53192744 | 64473 | 13: 53193885 |
| 64474 | 13: 53195323 | 64475 | 13: 53197934 | 64476 | 13: 53199665 | 64477 | 13: 53200513 | 64478 | 13: 53201494 |
| 64479 | 13: 53205894 | 64480 | 13: 53211390 | 64481 | 13: 53212642 | 64482 | 13: 53213246 | 64483 | 13: 53214256 |
| 64484 | 13: 53221957 | 64485 | 13: 53223771 | 64486 | 13: 53225335 | 64490 | 13: 53267383 | 64491 | 13: 53287399 |
| 64502 | 13: 53656779 | 64503 | 13: 53668817 | 64521 | 13: 54501975 | 64524 | 13: 54544791 | 64528 | 13: 54637805 |
| 64532 | 13: 54688808 | 64539 | 13: 54867617 | 64540 | 13: 54924099 | 64541 | 13: 54930460 | 64542 | 13: 54963877 |
| 64545 | 13: 55001775 | 64546 | 13: 55009029 | 64549 | 13: 55107919 | 64551 | 13: 55137013 | 64553 | 13: 55164494 |
| 64556 | 13: 55256797 | 64558 | 13: 55286529 | 64559 | 13: 55306487 | 64563 | 13: 55363256 | 64576 | 13: 55652395 |
| 64579 | 13: 55736298 | 64586 | 13: 55924074 | 64596 | 13: 56129806 | 64599 | 13: 56206409 | 64602 | 13: 56257733 |
| 64613 | 13: 56553088 | 64615 | 13: 56567143 | 64616 | 13: 56574066 | 64628 | 13: 56987530 | 64632 | 13: 57050196 |
| 64642 | 13: 57391199 | 64643 | 13: 57394025 | 64654 | 13: 57698221 | 64655 | 13: 57736295 | 64656 | 13: 57744564 |
| 64657 | 13: 57744624 | 64664 | 13: 57962862 | 64677 | 13: 58287555 | 64678 | 13: 58288391 | 64679 | 13: 58297881 |
| 64683 | 13: 58337666 | 64688 | 13: 58408741 | 64689 | 13: 58453137 | 64690 | 13: 58475365 | 64693 | 13: 58521548 |
| 64700 | 13: 58687733 | 64703 | 13: 58755844 | 64705 | 13: 58823450 | 64709 | 13: 58925642 | 64711 | 13: 58935983 |
| 64713 | 13: 58968219 | 64718 | 13: 58981092 | 64737 | 13: 59317532 | 64738 | 13: 59317573 | 64739 | 13: 59317600 |
| 64752 | 13: 59798919 | 64753 | 13: 59810522 | 64757 | 13: 59881900 | 64763 | 13: 59982505 | 64765 | 13: 60004397 |
| 64775 | 13: 60191346 | 64777 | 13: 60219559 | 64779 | 13: 60249957 | 64781 | 13: 60270738 | 64782 | 13: 60273459 |
| 64786 | 13: 60360621 | 64787 | 13: 60363893 | 64803 | 13: 60828808 | 64813 | 13: 61235720 | 64819 | 13: 61487750 |
| 64823 | 13: 61526661 | 64824 | 13: 61536675 | 64830 | 13: 61748180 | 64833 | 13: 61837125 | 64857 | 13: 62340129 |
| 64864 | 13: 62437505 | 64865 | 13: 62437664 | 64869 | 13: 62616409 | 64870 | 13: 62619495 | 64871 | 13: 62620895 |
| 64872 | 13: 62620917 | 64873 | 13: 62623230 | 64874 | 13: 62623246 | 64875 | 13: 62623260 | 64876 | 13: 62623271 |
| 64877 | 13: 62623277 | 64878 | 13: 62623296 | 64879 | 13: 62623326 | 64890 | 13: 62758151 | 64895 | 13: 62805225 |
| 64896 | 13: 62808252 | 64898 | 13: 62825728 | 64903 | 13: 62901932 | 64904 | 13: 62913802 | 64905 | 13: 62957243 |
| 64909 | 13: 63100717 | 64910 | 13: 63113668 | 64911 | 13: 63115815 | 64912 | 13: 63117671 | 64915 | 13: 63231676 |
| 64916 | 13: 63238472 | 64922 | 13: 63381167 | 64924 | 13: 63406383 | 64927 | 13: 63465271 | 64932 | 13: 63632674 |
| 64933 | 13: 63667387 | 64936 | 13: 63800029 | 64941 | 13: 63926705 | 64944 | 13: 63945489 | 64945 | 13: 63950962 |
| 64964 | 13: 64427414 | 64970 | 13: 64579641 | 64973 | 13: 64608490 | 64979 | 13: 64765574 | 64981 | 13: 64780081 |
| 64985 | 13: 64917447 | 64992 | 13: 65197131 | 64993 | 13: 65222405 | 64994 | 13: 65226257 | 64996 | 13: 65300967 |
| 65002 | 13: 65421703 | 65003 | 13: 65421742 | 65008 | 13: 65551485 | 65009 | 13: 65556446 | 65012 | 13: 65615123 |
| 65013 | 13: 65633719 | 65015 | 13: 65678606 | 65016 | 13: 65710639 | 65017 | 13: 65711755 | 65018 | 13: 65713224 |
| 65019 | 13: 65713273 | 65020 | 13: 65714024 | 65022 | 13: 65714678 | 65024 | 13: 65734511 | 65025 | 13: 65737670 |
| 65026 | 13: 65747364 | 65027 | 13: 65751601 | 65028 | 13: 65757675 | 65029 | 13: 65768152 | 65030 | 13: 65769345 |
| 65031 | 13: 65771689 | 65032 | 13: 65778554 | 65038 | 13: 65891788 | 65045 | 13: 66156547 | 65051 | 13: 66242964 |
| 65054 | 13: 66269753 | 65058 | 13: 66340843 | 65068 | 13: 66611398 | 65078 | 13: 66830214 | 65084 | 13: 66995569 |
| 65087 | 13: 67120106 | 65103 | 13: 67448820 | 65126 | 13: 68187572 | 65134 | 13: 68308181 | 65136 | 13: 68378513 |
| 65138 | 13: 68519206 | 65140 | 13: 68528941 | 65142 | 13: 68635086 | 65146 | 13: 68672438 | 65152 | 13: 68880703 |
| 65165 | 13: 69217542 | 65168 | 13: 69272129 | 65170 | 13: 69348741 | 65171 | 13: 69353425 | 65173 | 13: 69380590 |
| 65183 | 13: 69728600 | 65184 | 13: 69731770 | 65187 | 13: 69850661 | 65189 | 13: 69882420 | 65190 | 13: 69890851 |
| 65198 | 13: 70040165 | 65201 | 13: 70082692 | 65207 | 13: 70199117 | 65220 | 13: 70568959 | 65225 | 13: 70651503 |
| 65227 | 13: 70673685 | 65248 | 13: 71190284 | 65267 | 13: 71537155 | 65272 | 13: 71649810 | 65276 | 13: 71778746 |
| 65278 | 13: 71794746 | 65289 | 13: 72099141 | 65290 | 13: 72099160 | 65293 | 13: 72190824 | 65298 | 13: 72223856 |
| 65299 | 13: 72240445 | 65300 | 13: 72247438 | 65308 | 13: 72487250 | 65311 | 13: 72581815 | 65316 | 13: 72835577 |
| 65318 | 13: 72837581 | 65319 | 13: 72837712 | 65320 | 13: 72837736 | 65323 | 13: 72873627 | 65326 | 13: 72911160 |
| 65327 | 13: 72931732 | 65331 | 13: 72989401 | 65333 | 13: 73030191 | 65342 | 13: 73241000 | 65353 | 13: 73412480 |
| 65358 | 13: 73475168 | 65359 | 13: 73486255 | 65360 | 13: 73508637 | 65361 | 13: 73516499 | 65362 | 13: 73552702 |
| 65363 | 13: 73554377 | 65367 | 13: 73602251 | 65369 | 13: 73628006 | 65370 | 13: 73628032 | 65372 | 13: 73657151 |
| 65373 | 13: 73659243 | 65375 | 13: 73684336 | 65376 | 13: 73720189 | 65377 | 13: 73738431 | 65378 | 13: 73747745 |
| 65386 | 13: 74002990 | 65388 | 13: 74075881 | 65396 | 13: 74272611 | 65397 | 13: 74284172 | 65405 | 13: 74425124 |
| 65406 | 13: 74461134 | 65407 | 13: 74464989 | 65408 | 13: 74467020 | 65409 | 13: 74470296 | 65410 | 13: 74476473 |
| 65411 | 13: 74479132 | 65412 | 13: 74479161 | 65417 | 13: 74575589 | 65419 | 13: 74593675 | 65421 | 13: 74635628 |
| 65423 | 13: 74640625 | 65426 | 13: 74665130 | 65427 | 13: 74678375 | 65432 | 13: 74760409 | 65433 | 13: 74815833 |
| 65437 | 13: 74932791 | 65455 | 13: 75521831 | 65463 | 13: 75584984 | 65464 | 13: 75584987 | 65465 | 13: 75585044 |
| 65473 | 13: 75757004 | 65474 | 13: 75767512 | 65478 | 13: 75797537 | 65479 | 13: 75808921 | 65481 | 13: 75817367 |
| 65496 | 13: 76209741 | 65503 | 13: 76376258 | 65506 | 13: 76438473 | 65507 | 13: 76438492 | 65508 | 13: 76442151 |
| 65509 | 13: 76443128 | 65521 | 13: 76672357 | 65523 | 13: 76717525 | 65527 | 13: 76772665 | 65534 | 13: 76884649 |
| 65545 | 13: 77225860 | 65551 | 13: 77281427 | 65555 | 13: 77332710 | 65557 | 13: 77387866 | 65560 | 13: 77423447 |
| 65561 | 13: 77443501 | 65572 | 13: 77715514 | 65574 | 13: 77719500 | 65576 | 13: 77810786 | 65579 | 13: 77851241 |
| 65580 | 13: 77856986 | 65584 | 13: 78019495 | 65589 | 13: 78097466 | 65590 | 13: 78097543 | 65591 | 13: 78097572 |
| 65595 | 13: 78246967 | 65600 | 13: 78323978 | 65601 | 13: 78327563 | 65611 | 13: 78606738 | 65620 | 13: 78732308 |
| 65628 | 13: 78862443 | 65630 | 13: 78895292 | 65636 | 13: 79010034 | 65646 | 13: 79138574 | 65653 | 13: 79289518 |
| 65663 | 13: 79372632 | 65664 | 13: 79378945 | 65670 | 13: 79439113 | 65746 | 13: 80685975 | 65753 | 13: 80868488 |
| 65754 | 13: 80869352 | 65775 | 13: 81361992 | 65780 | 13: 81441602 | 65789 | 13: 81578319 | 65793 | 13: 81659411 |
| 65796 | 13: 81695713 | 65809 | 13: 81975437 | 65811 | 13: 82042702 | 65857 | 13: 82944108 | 65859 | 13: 82944150 |
| 65870 | 13: 83194337 | 65885 | 14: 260388 | 65890 | 14: 344042 | 65892 | 14: 428398 | 65906 | 14: 1024267 |
| 65907 | 14: 1025055 | 65908 | 14: 1033297 | 65909 | 14: 1036134 | 65910 | 14: 1039518 | 65911 | 14: 1044777 |
| 65914 | 14: 1153408 | 65916 | 14: 1188418 | 65919 | 14: 1314992 | 65923 | 14: 1388615 | 65924 | 14: 1394179 |
| 65928 | 14: 1554543 | 65930 | 14: 1658546 | 65931 | 14: 1670892 | 65932 | 14: 1671183 | 65933 | 14: 1671573 |
| 65934 | 14: 1671580 | 65935 | 14: 1681783 | 65936 | 14: 1702340 | 65940 | 14: 1785051 | 65941 | 14: 1798788 |
| 65942 | 14: 1799618 | 65943 | 14: 1799673 | 65944 | 14: 1800049 | 65946 | 14: 1844464 | 65947 | 14: 1854457 |
| 65950 | 14: 1926589 | 65951 | 14: 1946346 | 65966 | 14: 2415920 | 65968 | 14: 2500509 | 65973 | 14: 2669589 |
| 65982 | 14: 2854696 | 65988 | 14: 3015800 | 65989 | 14: 3040833 | 65990 | 14: 3040885 | 65994 | 14: 3097950 |
| 65998 | 14: 3147254 | 66005 | 14: 3206294 | 66006 | 14: 3212884 | 66007 | 14: 3218403 | 66008 | 14: 3218478 |
| 66009 | 14: 3218557 | 66010 | 14: 3221269 | 66011 | 14: 3221338 | 66012 | 14: 3239650 | 66013 | 14: 3239696 |
| 66014 | 14: 3239752 | 66015 | 14: 3239754 | 66016 | 14: 3242336 | 66017 | 14: 3246999 | 66018 | 14: 3247042 |
| 66019 | 14: 3247052 | 66020 | 14: 3278835 | 66021 | 14: 3278838 | 66022 | 14: 3284409 | 66023 | 14: 3284439 |
| 66024 | 14: 3298378 | 66025 | 14: 3305527 | 66026 | 14: 3308936 | 66028 | 14: 3336128 | 66029 | 14: 3336263 |
| 66034 | 14: 3386889 | 66036 | 14: 3433819 | 66038 | 14: 3453301 | 66041 | 14: 3471847 | 66080 | 14: 4216507 |
| 66082 | 14: 4228026 | 66083 | 14: 4228352 | 66085 | 14: 4232919 | 66086 | 14: 4233543 | 66113 | 14: 4912069 |
| 66118 | 14: 5004897 | 66178 | 14: 5998302 | 66185 | 14: 6071169 | 66191 | 14: 6163665 | 66192 | 14: 6174543 |
| 66203 | 14: 6285204 | 66206 | 14: 6295145 | 66207 | 14: 6303201 | 66212 | 14: 6388522 | 66214 | 14: 6472930 |
| 66237 | 14: 6859614 | 66248 | 14: 7064392 | 66250 | 14: 7109542 | 66252 | 14: 7127144 | 66269 | 14: 7497044 |
| 66274 | 14: 7545925 | 66275 | 14: 7545927 | 66276 | 14: 7545936 | 66280 | 14: 7626758 | 66294 | 14: 7895151 |
| 66300 | 14: 7989733 | 66311 | 14: 8058621 | 66312 | 14: 8058649 | 66316 | 14: 8179389 | 66321 | 14: 8301355 |
| 66323 | 14: 8312764 | 66327 | 14: 8394739 | 66328 | 14: 8416607 | 66329 | 14: 8424727 | 66331 | 14: 8441111 |
| 66333 | 14: 8445298 | 66337 | 14: 8540399 | 66338 | 14: 8551277 | 66340 | 14: 8561525 | 66341 | 14: 8566274 |
| 66343 | 14: 8579200 | 66346 | 14: 8601680 | 66347 | 14: 8601681 | 66349 | 14: 8629638 | 66353 | 14: 8664840 |
| 66354 | 14: 8664847 | 66356 | 14: 8672236 | 66359 | 14: 8707161 | 66363 | 14: 8798924 | 66375 | 14: 9035591 |
| 66378 | 14: 9063069 | 66379 | 14: 9064094 | 66380 | 14: 9064111 | 66381 | 14: 9064154 | 66382 | 14: 9064258 |
| 66384 | 14: 9095754 | 66386 | 14: 9121381 | 66387 | 14: 9140334 | 66388 | 14: 9140361 | 66389 | 14: 9140418 |
| 66390 | 14: 9141498 | 66394 | 14: 9188175 | 66400 | 14: 9361706 | 66402 | 14: 9382539 | 66403 | 14: 9399137 |
| 66404 | 14: 9401991 | 66430 | 14: 9976682 | 66431 | 14: 9976704 | 66432 | 14: 9976709 | 66434 | 14: 10038814 |
| 66438 | 14: 10102756 | 66440 | 14: 10152585 | 66443 | 14: 10280749 | 66451 | 14: 10486501 | 66455 | 14: 10530506 |
| 66457 | 14: 10557006 | 66458 | 14: 10566461 | 66459 | 14: 10577632 | 66462 | 14: 10706026 | 66464 | 14: 10756790 |
| 66465 | 14: 10764173 | 66466 | 14: 10788680 | 66467 | 14: 10801527 | 66468 | 14: 10805790 | 66469 | 14: 10807806 |
| 66470 | 14: 10816880 | 66474 | 14: 10893686 | 66476 | 14: 10914761 | 66479 | 14: 10947558 | 66481 | 14: 11009369 |
| 66484 | 14: 11088275 | 66492 | 14: 11252807 | 66504 | 14: 11411289 | 66505 | 14: 11444087 | 66510 | 14: 11540331 |
| 66512 | 14: 11568465 | 66513 | 14: 11575517 | 66516 | 14: 11596900 | 66517 | 14: 11657154 | 66526 | 14: 11763629 |
| 66533 | 14: 11962212 | 66541 | 14: 12136166 | 66546 | 14: 12207418 | 66551 | 14: 12292359 | 66552 | 14: 12293976 |
| 66554 | 14: 12315574 | 66558 | 14: 12384353 | 66559 | 14: 12388961 | 66560 | 14: 12393160 | 66564 | 14: 12514540 |
| 66578 | 14: 12695561 | 66579 | 14: 12710347 | 66593 | 14: 13094541 | 66595 | 14: 13178679 | 66618 | 14: 13920366 |
| 66623 | 14: 14023534 | 66628 | 14: 14069858 | 66631 | 14: 14135529 | 66636 | 14: 14326793 | 66646 | 14: 14560727 |
| 66649 | 14: 14707903 | 66651 | 14: 14728697 | 66652 | 14: 14744411 | 66656 | 14: 14812773 | 66657 | 14: 14823932 |
| 66662 | 14: 14952020 | 66664 | 14: 15013585 | 66666 | 14: 15072256 | 66668 | 14: 15100414 | 66675 | 14: 15318937 |
| 66677 | 14: 15351351 | 66678 | 14: 15369492 | 66682 | 14: 15454870 | 66689 | 14: 15529862 | 66704 | 14: 15827253 |
| 66710 | 14: 15926072 | 66712 | 14: 15954837 | 66715 | 14: 15972218 | 66723 | 14: 16143522 | 66725 | 14: 16177691 |
| 66730 | 14: 16258523 | 66734 | 14: 16293114 | 66737 | 14: 16436394 | 66738 | 14: 16442429 | 66739 | 14: 16450949 |
| 66741 | 14: 16479769 | 66742 | 14: 16493894 | 66752 | 14: 16722564 | 66753 | 14: 16790460 | 66754 | 14: 16817518 |
| 66756 | 14: 16852507 | 66761 | 14: 16906633 | 66762 | 14: 16939530 | 66763 | 14: 16939542 | 66764 | 14: 16939598 |
| 66765 | 14: 16939628 | 66767 | 14: 16969260 | 66771 | 14: 17041215 | 66778 | 14: 17280279 | 66798 | 14: 17770696 |
| 66799 | 14: 17770830 | 66801 | 14: 17799708 | 66802 | 14: 17816110 | 66803 | 14: 17843985 | 66836 | 14: 18601570 |
| 66849 | 14: 18880871 | 66860 | 14: 19181912 | 66865 | 14: 19283893 | 66869 | 14: 19432077 | 66881 | 14: 19699930 |
| 66882 | 14: 19743977 | 66885 | 14: 19813853 | 66890 | 14: 19975802 | 66893 | 14: 19991875 | 66927 | 14: 20847256 |
| 66930 | 14: 20893054 | 66931 | 14: 20896371 | 66932 | 14: 20913913 | 66935 | 14: 20978169 | 66937 | 14: 21048592 |
| 66958 | 14: 21485350 | 66963 | 14: 21604476 | 66974 | 14: 21869418 | 66977 | 14: 21931720 | 66979 | 14: 21998392 |
| 66984 | 14: 22161961 | 66995 | 14: 22407071 | 66996 | 14: 22407076 | 67077 | 14: 22732607 | 67093 | 14: 22882106 |
| 67095 | 14: 22886422 | 67173 | 14: 23215974 | 67345 | 14: 23539160 | 67346 | 14: 23539175 | 67347 | 14: 23539193 |
| 67348 | 14: 23539196 | 67349 | 14: 23539229 | 67424 | 14: 23981200 | 67428 | 14: 24041313 | 67443 | 14: 24389626 |
| 67445 | 14: 24408833 | 67514 | 14: 25111427 | 67519 | 14: 25203874 | 67520 | 14: 25205074 | 67525 | 14: 25258907 |
| 67529 | 14: 25432157 | 67561 | 14: 25864255 | 67565 | 14: 25923336 | 67566 | 14: 25923373 | 67568 | 14: 25985806 |
| 67570 | 14: 25990906 | 67571 | 14: 26012359 | 67573 | 14: 26052398 | 67575 | 14: 26065694 | 67580 | 14: 26140450 |
| 67581 | 14: 26140990 | 67582 | 14: 26141962 | 67583 | 14: 26143779 | 67585 | 14: 26163203 | 67586 | 14: 26170631 |
| 67587 | 14: 26175012 | 67588 | 14: 26176715 | 67590 | 14: 26182412 | 67591 | 14: 26182943 | 67593 | 14: 26184111 |
| 67594 | 14: 26184775 | 67595 | 14: 26185527 | 67596 | 14: 26186351 | 67597 | 14: 26190407 | 67598 | 14: 26191236 |
| 67600 | 14: 26193748 | 67601 | 14: 26194782 | 67602 | 14: 26195880 | 67603 | 14: 26197356 | 67604 | 14: 26209375 |
| 67605 | 14: 26210727 | 67606 | 14: 26212648 | 67607 | 14: 26216203 | 67608 | 14: 26225153 | 67610 | 14: 26246778 |
| 67611 | 14: 26271742 | 67614 | 14: 26308735 | 67617 | 14: 26333506 | 67620 | 14: 26432685 | 67626 | 14: 26633301 |
| 67632 | 14: 26717685 | 67648 | 14: 27181625 | 67649 | 14: 27210120 | 67650 | 14: 27233742 | 67670 | 14: 27952519 |
| 67673 | 14: 28069983 | 67685 | 14: 28479844 | 67691 | 14: 28620485 | 67692 | 14: 28622173 | 67693 | 14: 28624764 |
| 67697 | 14: 28758622 | 67715 | 14: 29296020 | 67725 | 14: 29459640 | 67740 | 14: 29698665 | 67748 | 14: 29894372 |
| 67749 | 14: 29989315 | 67752 | 14: 30012586 | 67767 | 14: 30342088 | 67768 | 14: 30349359 | 67769 | 14: 30356890 |
| 67778 | 14: 30465481 | 67781 | 14: 30499744 | 67794 | 14: 30690571 | 67797 | 14: 30764962 | 67798 | 14: 30811197 |
| 67808 | 14: 31180467 | 67819 | 14: 31555900 | 67820 | 14: 31555992 | 67826 | 14: 31641997 | 67833 | 14: 31874739 |
| 67836 | 14: 31944000 | 67844 | 14: 32203109 | 67866 | 14: 32398203 | 67886 | 14: 32981178 | 67890 | 14: 33060155 |
| 67896 | 14: 33143197 | 67897 | 14: 33143201 | 67900 | 14: 33275732 | 67906 | 14: 33326342 | 67907 | 14: 33342080 |
| 67912 | 14: 33449262 | 67913 | 14: 33458276 | 67919 | 14: 33529983 | 67930 | 14: 33731278 | 67932 | 14: 33839737 |
| 67938 | 14: 33897507 | 67939 | 14: 33921705 | 67942 | 14: 33981693 | 67947 | 14: 34164312 | 67953 | 14: 34301611 |
| 67959 | 14: 34528656 | 67975 | 14: 35058048 | 68018 | 14: 36043204 | 68028 | 14: 36299508 | 68035 | 14: 36373476 |
| 68052 | 14: 36852980 | 68071 | 14: 37488132 | 68084 | 14: 37849675 | 68085 | 14: 37852302 | 68089 | 14: 37925483 |
| 68097 | 14: 38188466 | 68115 | 14: 38814239 | 68116 | 14: 38815880 | 68117 | 14: 38822128 | 68118 | 14: 38822260 |
| 68141 | 14: 39497949 | 68155 | 14: 39876130 | 68156 | 14: 39966401 | 68160 | 14: 40176632 | 68161 | 14: 40176648 |
| 68162 | 14: 40176909 | 68163 | 14: 40176917 | 68164 | 14: 40176996 | 68165 | 14: 40177014 | 68166 | 14: 40177030 |
| 68167 | 14: 40177236 | 68168 | 14: 40177299 | 68179 | 14: 40478861 | 68203 | 14: 41361918 | 68205 | 14: 41438833 |
| 68208 | 14: 41573733 | 68214 | 14: 41689230 | 68217 | 14: 41730144 | 68218 | 14: 41737864 | 68242 | 14: 42286395 |
| 68254 | 14: 42523669 | 68264 | 14: 42805270 | 68273 | 14: 43158811 | 68280 | 14: 43440082 | 68288 | 14: 43769450 |
| 68307 | 14: 44309555 | 68309 | 14: 44430721 | 68318 | 14: 44636417 | 68322 | 14: 44676672 | 68323 | 14: 44676728 |
| 68324 | 14: 44677183 | 68326 | 14: 44690793 | 68327 | 14: 44724666 | 68329 | 14: 44762792 | 68331 | 14: 44776625 |
| 68334 | 14: 44843603 | 68335 | 14: 44854809 | 68336 | 14: 44856580 | 68340 | 14: 44966227 | 68344 | 14: 44988177 |
| 68345 | 14: 45015405 | 68346 | 14: 45018776 | 68347 | 14: 45022201 | 68348 | 14: 45041057 | 68356 | 14: 45253129 |
| 68357 | 14: 45264773 | 68362 | 14: 45445260 | 68378 | 14: 45823929 | 68379 | 14: 45842554 | 68380 | 14: 45845229 |
| 68387 | 14: 45960737 | 68392 | 14: 46043332 | 68395 | 14: 46074189 | 68416 | 14: 46394434 | 68419 | 14: 46425925 |
| 68425 | 14: 46627739 | 68427 | 14: 46701293 | 68437 | 14: 46893352 | 68447 | 14: 47088081 | 68477 | 14: 48079880 |
| 68486 | 14: 48324535 | 68488 | 14: 48324675 | 68491 | 14: 48337571 | 68505 | 14: 48683313 | 68508 | 14: 48746367 |
| 68510 | 14: 48777365 | 68531 | 14: 49324283 | 68534 | 14: 49446616 | 68535 | 14: 49446636 | 68557 | 14: 50162370 |
| 68562 | 14: 50269979 | 68571 | 14: 50641123 | 68586 | 14: 51157346 | 68593 | 14: 51380992 | 68594 | 14: 51381093 |
| 68599 | 14: 51513649 | 68609 | 14: 51705820 | 68618 | 14: 51969375 | 68621 | 14: 52011701 | 68622 | 14: 52087941 |
| 68624 | 14: 52117812 | 68626 | 14: 52174077 | 68627 | 14: 52174084 | 68628 | 14: 52176416 | 68629 | 14: 52176503 |
| 68630 | 14: 52181003 | 68631 | 14: 52193756 | 68632 | 14: 52206425 | 68634 | 14: 52240902 | 68635 | 14: 52240959 |
| 68636 | 14: 52272882 | 68637 | 14: 52274310 | 68647 | 14: 52598698 | 68666 | 14: 53395458 | 68667 | 14: 53433645 |
| 68678 | 14: 53716855 | 68694 | 14: 54155951 | 68699 | 14: 54289749 | 68703 | 14: 54322218 | 68704 | 14: 54323172 |
| 68705 | 14: 54324711 | 68706 | 14: 54326865 | 68707 | 14: 54328568 | 68708 | 14: 54329372 | 68709 | 14: 54330950 |
| 68710 | 14: 54331798 | 68711 | 14: 54332484 | 68712 | 14: 54333187 | 68713 | 14: 54334454 | 68714 | 14: 54335896 |
| 68715 | 14: 54340206 | 68718 | 14: 54366857 | 68719 | 14: 54375801 | 68722 | 14: 54397201 | 68723 | 14: 54441985 |
| 68725 | 14: 54458565 | 68732 | 14: 54625281 | 68745 | 14: 54834484 | 68749 | 14: 55039582 | 68751 | 14: 55074676 |
| 68756 | 14: 55222131 | 68757 | 14: 55226008 | 68758 | 14: 55230280 | 68759 | 14: 55230389 | 68760 | 14: 55283357 |
| 68769 | 14: 55484731 | 68780 | 14: 55685950 | 68789 | 14: 56225330 | 68790 | 14: 56256482 | 68791 | 14: 56276255 |
| 68799 | 14: 56506951 | 68803 | 14: 56545284 | 68804 | 14: 56555253 | 68808 | 14: 56652939 | 68816 | 14: 56827541 |
| 68831 | 14: 57332067 | 68832 | 14: 57346169 | 68833 | 14: 57350713 | 68834 | 14: 57366372 | 68835 | 14: 57383485 |
| 68839 | 14: 57448890 | 68840 | 14: 57459060 | 68842 | 14: 57536798 | 68846 | 14: 57585046 | 68851 | 14: 57728888 |
| 68852 | 14: 57728921 | 68853 | 14: 57779780 | 68854 | 14: 57796975 | 68856 | 14: 57816918 | 68859 | 14: 57922597 |
| 68860 | 14: 57935303 | 68864 | 14: 58056243 | 68868 | 14: 58089515 | 68874 | 14: 58220993 | 68885 | 14: 58495696 |
| 68886 | 14: 58510271 | 68888 | 14: 58533542 | 68892 | 14: 58668510 | 68905 | 14: 58947933 | 68910 | 14: 59026237 |
| 68912 | 14: 59043151 | 68920 | 14: 59231026 | 68947 | 14: 59935092 | 68953 | 14: 60142373 | 68957 | 14: 60181604 |
| 68958 | 14: 60181624 | 68964 | 14: 60282711 | 68965 | 14: 60298957 | 68967 | 14: 60335923 | 68969 | 14: 60400617 |
| 68973 | 14: 60573159 | 68977 | 14: 60646587 | 68978 | 14: 60646600 | 68983 | 14: 60827415 | 68990 | 14: 60986477 |
| 68994 | 14: 61146297 | 68996 | 14: 61230816 | 68997 | 14: 61230819 | 69001 | 14: 61287942 | 69002 | 14: 61313591 |
| 69003 | 14: 61315627 | 69007 | 14: 61411151 | 69008 | 14: 61412641 | 69009 | 14: 61412766 | 69010 | 14: 61412805 |
| 69011 | 14: 61414085 | 69014 | 14: 61513794 | 69028 | 14: 61777801 | 69033 | 14: 61849353 | 69040 | 14: 61948505 |
| 69044 | 14: 62039648 | 69046 | 14: 62081343 | 69049 | 14: 62134019 | 69052 | 14: 62144762 | 69053 | 14: 62144767 |
| 69054 | 14: 62149426 | 69060 | 14: 62299231 | 69068 | 14: 62442888 | 69073 | 14: 62565653 | 69078 | 14: 62687349 |
| 69088 | 14: 62815477 | 69090 | 14: 62881003 | 69100 | 14: 63151822 | 69103 | 14: 63198642 | 69104 | 14: 63225917 |
| 69113 | 14: 63395803 | 69128 | 14: 63850266 | 69131 | 14: 63928813 | 69146 | 14: 64220537 | 69149 | 14: 64336877 |
| 69150 | 14: 64339120 | 69151 | 14: 64353146 | 69156 | 14: 64514888 | 69194 | 14: 65705048 | 69201 | 14: 65784254 |
| 69206 | 14: 65890326 | 69211 | 14: 66085117 | 69212 | 14: 66121609 | 69213 | 14: 66141319 | 69221 | 14: 66266296 |
| 69222 | 14: 66266410 | 69224 | 14: 66350819 | 69225 | 14: 66350850 | 69233 | 14: 66424408 | 69237 | 14: 66604661 |
| 69240 | 14: 66662602 | 69241 | 14: 66669902 | 69242 | 14: 66669945 | 69247 | 14: 66766263 | 69263 | 14: 67192810 |
| 69265 | 14: 67258930 | 69268 | 14: 67326499 | 69270 | 14: 67366402 | 69274 | 14: 67469984 | 69275 | 14: 67472732 |
| 69276 | 14: 67473297 | 69277 | 14: 67473813 | 69278 | 14: 67478743 | 69279 | 14: 67483782 | 69280 | 14: 67495613 |
| 69284 | 14: 67550636 | 69287 | 14: 67603655 | 69290 | 14: 67630731 | 69292 | 14: 67655532 | 69293 | 14: 67660678 |
| 69299 | 14: 67907866 | 69318 | 14: 68256914 | 69323 | 14: 68358639 | 69355 | 14: 69386387 | 69366 | 14: 69774142 |
| 69367 | 14: 69787949 | 69368 | 14: 69802911 | 69371 | 14: 69913935 | 69373 | 14: 69940920 | 69375 | 14: 70002759 |
| 69381 | 14: 70075613 | 69385 | 14: 70170918 | 69386 | 14: 70171506 | 69388 | 14: 70172986 | 69392 | 14: 70218397 |
| 69398 | 14: 70356280 | 69401 | 14: 70440463 | 69403 | 14: 70472924 | 69412 | 14: 70696424 | 69418 | 14: 70903085 |
| 69420 | 14: 70964025 | 69440 | 14: 71594264 | 69447 | 14: 71762783 | 69459 | 14: 72082157 | 69485 | 14: 72739791 |
| 69489 | 14: 72764165 | 69499 | 14: 73108865 | 69504 | 14: 73206090 | 69507 | 14: 73232071 | 69510 | 14: 73246685 |
| 69516 | 14: 73444151 | 69525 | 14: 73731378 | 69529 | 14: 73806578 | 69578 | 14: 75097229 | 69579 | 14: 75097255 |
| 69582 | 14: 75218040 | 69586 | 14: 75379695 | 69587 | 14: 75379698 | 69588 | 14: 75379711 | 69592 | 14: 75591751 |
| 69596 | 14: 75706688 | 69611 | 14: 76100504 | 69612 | 14: 76100657 | 69615 | 14: 76128821 | 69616 | 14: 76129624 |
| 69617 | 14: 76132580 | 69618 | 14: 76138175 | 69619 | 14: 76141064 | 69621 | 14: 76142867 | 69622 | 14: 76144495 |
| 69625 | 14: 76178839 | 69626 | 14: 76178890 | 69628 | 14: 76195852 | 69629 | 14: 76199476 | 69630 | 14: 76207317 |
| 69635 | 14: 76328357 | 69637 | 14: 76366242 | 69646 | 14: 76503558 | 69653 | 14: 76612609 | 69655 | 14: 76770661 |
| 69656 | 14: 76771798 | 69659 | 14: 76845625 | 69661 | 14: 76923277 | 69663 | 14: 76945033 | 69664 | 14: 76964292 |
| 69670 | 14: 77099484 | 69671 | 14: 77174586 | 69672 | 14: 77206279 | 69681 | 14: 77471278 | 69682 | 14: 77471456 |
| 69684 | 14: 77512276 | 69712 | 14: 78249703 | 69713 | 14: 78249764 | 69714 | 14: 78249780 | 69720 | 14: 78305043 |
| 69721 | 14: 78306690 | 69722 | 14: 78312623 | 69725 | 14: 78390822 | 69726 | 14: 78392259 | 69727 | 14: 78393807 |
| 69729 | 14: 78454550 | 69738 | 14: 78651480 | 69747 | 14: 78858840 | 69755 | 14: 79091368 | 69781 | 14: 79790786 |
| 69787 | 14: 79936016 | 69795 | 14: 80077176 | 69796 | 14: 80078158 | 69809 | 14: 80313052 | 69832 | 14: 80855743 |
| 69848 | 14: 81221549 | 69849 | 14: 81264509 | 69850 | 14: 81267100 | 69851 | 14: 81267190 | 69852 | 14: 81267746 |
| 69853 | 14: 81295824 | 69854 | 14: 81295958 | 69861 | 14: 81372244 | 69862 | 14: 81379555 | 69863 | 14: 81392217 |
| 69872 | 14: 81555021 | 69873 | 14: 81583416 | 69876 | 14: 81598835 | 69879 | 14: 81634869 | 69881 | 14: 81683735 |
| 69882 | 14: 81698012 | 69883 | 14: 81701580 | 69887 | 14: 81727910 | 69893 | 14: 81835309 | 69895 | 14: 81857929 |
| 69897 | 14: 81940278 | 69899 | 14: 81972242 | 69903 | 14: 82015406 | 69904 | 14: 82016702 | 69905 | 14: 82026443 |
| 69909 | 14: 82091345 | 69933 | 15: 556424 | 69946 | 15: 1148939 | 69948 | 15: 1192605 | 69953 | 15: 1229858 |
| 69961 | 15: 1387083 | 69962 | 15: 1439384 | 70009 | 15: 2844618 | 70063 | 15: 3928859 | 70079 | 15: 4264863 |
| 70083 | 15: 4294447 | 70092 | 15: 4497670 | 70099 | 15: 4629275 | 70119 | 15: 4985484 | 70122 | 15: 5160844 |
| 70123 | 15: 5160845 | 70124 | 15: 5160859 | 70125 | 15: 5160876 | 70126 | 15: 5160956 | 70128 | 15: 5219937 |
| 70129 | 15: 5234380 | 70130 | 15: 5296961 | 70282 | 15: 5477594 | 70286 | 15: 5550405 | 70289 | 15: 5646426 |
| 70292 | 15: 5686808 | 70293 | 15: 5686891 | 70294 | 15: 5686893 | 70297 | 15: 5779163 | 70300 | 15: 5933216 |
| 70306 | 15: 6041257 | 70320 | 15: 6451524 | 70323 | 15: 6555645 | 70324 | 15: 6579715 | 70329 | 15: 6707584 |
| 70374 | 15: 7579129 | 70391 | 15: 8102324 | 70413 | 15: 8544147 | 70414 | 15: 8547793 | 70425 | 15: 8919907 |
| 70434 | 15: 9119501 | 70444 | 15: 9307296 | 70445 | 15: 9307388 | 70446 | 15: 9308875 | 70449 | 15: 9342376 |
| 70461 | 15: 9653267 | 70462 | 15: 9653311 | 70466 | 15: 9752248 | 70491 | 15: 10716741 | 70493 | 15: 10740715 |
| 70496 | 15: 10827424 | 70505 | 15: 11236303 | 70520 | 15: 11770301 | 70529 | 15: 12167647 | 70540 | 15: 12557589 |
| 70574 | 15: 13886436 | 70586 | 15: 14183988 | 70588 | 15: 14191992 | 70591 | 15: 14203309 | 70594 | 15: 14260966 |
| 70599 | 15: 14448696 | 70600 | 15: 14456572 | 70601 | 15: 14458763 | 70602 | 15: 14459519 | 70603 | 15: 14461707 |
| 70604 | 15: 14462057 | 70605 | 15: 14462078 | 70606 | 15: 14463666 | 70612 | 15: 14705603 | 70617 | 15: 14911197 |
| 70623 | 15: 15068168 | 70624 | 15: 15068209 | 70631 | 15: 15245724 | 70643 | 15: 15417315 | 70644 | 15: 15417341 |
| 70645 | 15: 15417537 | 70646 | 15: 15417584 | 70648 | 15: 15474272 | 70649 | 15: 15505264 | 70651 | 15: 15537455 |
| 70656 | 15: 15691423 | 70657 | 15: 15691442 | 70658 | 15: 15715228 | 70661 | 15: 15789950 | 70664 | 15: 15846394 |
| 70665 | 15: 15857275 | 70666 | 15: 15867538 | 70667 | 15: 15882003 | 70668 | 15: 15887057 | 70671 | 15: 15896498 |
| 70672 | 15: 15896623 | 70673 | 15: 15896626 | 70676 | 15: 15899461 | 70678 | 15: 15941852 | 70685 | 15: 16025726 |
| 70686 | 15: 16034972 | 70688 | 15: 16066600 | 70689 | 15: 16070995 | 70690 | 15: 16086621 | 70691 | 15: 16086825 |
| 70692 | 15: 16090802 | 70693 | 15: 16095590 | 70694 | 15: 16098140 | 70697 | 15: 16106288 | 70698 | 15: 16106299 |
| 70699 | 15: 16106352 | 70700 | 15: 16106429 | 70701 | 15: 16106956 | 70703 | 15: 16181648 | 70720 | 15: 16783559 |
| 70722 | 15: 16876160 | 70725 | 15: 17015197 | 70726 | 15: 17021701 | 70727 | 15: 17023074 | 70728 | 15: 17023080 |
| 70729 | 15: 17035605 | 70730 | 15: 17045413 | 70731 | 15: 17050219 | 70732 | 15: 17051620 | 70734 | 15: 17079996 |
| 70737 | 15: 17110054 | 70739 | 15: 17151448 | 70740 | 15: 17156993 | 70742 | 15: 17174949 | 70743 | 15: 17174997 |
| 70744 | 15: 17178030 | 70748 | 15: 17216169 | 70749 | 15: 17225710 | 70752 | 15: 17272317 | 70771 | 15: 17881167 |
| 70772 | 15: 17905884 | 70773 | 15: 17907768 | 70774 | 15: 17907803 | 70776 | 15: 17984528 | 70780 | 15: 18132345 |
| 70788 | 15: 18340406 | 70789 | 15: 18340444 | 70790 | 15: 18340452 | 70791 | 15: 18340495 | 70792 | 15: 18340522 |
| 70800 | 15: 18582994 | 70828 | 15: 19344195 | 70852 | 15: 20040778 | 70853 | 15: 20041510 | 70855 | 15: 20059908 |
| 70861 | 15: 20159086 | 70862 | 15: 20208131 | 70863 | 15: 20239028 | 70864 | 15: 20251395 | 70874 | 15: 20506266 |
| 70898 | 15: 20943851 | 70900 | 15: 20983899 | 70904 | 15: 21079829 | 70909 | 15: 21144577 | 70910 | 15: 21222779 |
| 70915 | 15: 21328250 | 70916 | 15: 21331201 | 70917 | 15: 21344247 | 70927 | 15: 21713133 | 70928 | 15: 21715125 |
| 70930 | 15: 21724458 | 70937 | 15: 21944261 | 70938 | 15: 21955334 | 70939 | 15: 21959233 | 70940 | 15: 21980185 |
| 70944 | 15: 22109125 | 70947 | 15: 22219010 | 70950 | 15: 22224493 | 70951 | 15: 22255512 | 70959 | 15: 22397563 |
| 70988 | 15: 23032442 | 70992 | 15: 23140676 | 70995 | 15: 23230388 | 71002 | 15: 23361381 | 71003 | 15: 23382217 |
| 71006 | 15: 23428462 | 71007 | 15: 23428524 | 71008 | 15: 23428581 | 71009 | 15: 23495909 | 71023 | 15: 23729866 |
| 71024 | 15: 23732149 | 71059 | 15: 23865919 | 71072 | 15: 24238065 | 71074 | 15: 24278519 | 71083 | 15: 24394620 |
| 71084 | 15: 24406959 | 71103 | 15: 24778258 | 71104 | 15: 24778597 | 71105 | 15: 24783092 | 71145 | 15: 25774695 |
| 71150 | 15: 25820329 | 71157 | 15: 25920672 | 71166 | 15: 26130885 | 71170 | 15: 26196504 | 71175 | 15: 26276504 |
| 71176 | 15: 26297137 | 71192 | 15: 26653665 | 71199 | 15: 26809489 | 71211 | 15: 27151330 | 71219 | 15: 27430624 |
| 71223 | 15: 27474421 | 71226 | 15: 27503920 | 71228 | 15: 27522579 | 71230 | 15: 27581168 | 71236 | 15: 27795197 |
| 71240 | 15: 27909371 | 71243 | 15: 27975706 | 71246 | 15: 28011102 | 71247 | 15: 28029857 | 71248 | 15: 28029959 |
| 71249 | 15: 28047023 | 71251 | 15: 28062942 | 71256 | 15: 28137955 | 71257 | 15: 28138113 | 71260 | 15: 28217846 |
| 71261 | 15: 28233069 | 71264 | 15: 28261207 | 71276 | 15: 28502744 | 71285 | 15: 28668348 | 71286 | 15: 28669780 |
| 71287 | 15: 28674486 | 71288 | 15: 28675386 | 71289 | 15: 28678307 | 71290 | 15: 28679179 | 71292 | 15: 28683279 |
| 71293 | 15: 28687052 | 71294 | 15: 28689278 | 71304 | 15: 28889609 | 71305 | 15: 28903033 | 71306 | 15: 28903036 |
| 71307 | 15: 28904856 | 71311 | 15: 28935265 | 71314 | 15: 28955129 | 71315 | 15: 28957203 | 71316 | 15: 28995849 |
| 71318 | 15: 29029067 | 71320 | 15: 29051391 | 71327 | 15: 29217795 | 71331 | 15: 29377994 | 71338 | 15: 29599939 |
| 71341 | 15: 29669861 | 71342 | 15: 29691482 | 71343 | 15: 29695545 | 71344 | 15: 29708525 | 71358 | 15: 30156144 |
| 71359 | 15: 30182678 | 71372 | 15: 30495429 | 71376 | 15: 30550572 | 71378 | 15: 30571590 | 71379 | 15: 30592484 |
| 71381 | 15: 30681136 | 71387 | 15: 30887853 | 71395 | 15: 31076884 | 71398 | 15: 31119240 | 71423 | 15: 31752349 |
| 71426 | 15: 31762344 | 71427 | 15: 31762695 | 71428 | 15: 31762716 | 71429 | 15: 31762815 | 71430 | 15: 31766858 |
| 71431 | 15: 31772217 | 71432 | 15: 31772367 | 71433 | 15: 31772508 | 71435 | 15: 31789881 | 71439 | 15: 31967339 |
| 71447 | 15: 32184560 | 71448 | 15: 32184561 | 71455 | 15: 32326526 | 71457 | 15: 32408675 | 71458 | 15: 32415848 |
| 71473 | 15: 32775518 | 71475 | 15: 32857841 | 71479 | 15: 32885957 | 71481 | 15: 32912616 | 71482 | 15: 32919523 |
| 71487 | 15: 33030880 | 71493 | 15: 33140018 | 71495 | 15: 33140162 | 71496 | 15: 33143682 | 71499 | 15: 33200022 |
| 71502 | 15: 33313146 | 71505 | 15: 33357725 | 71513 | 15: 33683094 | 71522 | 15: 33855059 | 71546 | 15: 34227669 |
| 71554 | 15: 34378606 | 71558 | 15: 34416916 | 71563 | 15: 34514270 | 71564 | 15: 34527315 | 71572 | 15: 34677976 |
| 71574 | 15: 34706081 | 71576 | 15: 34775584 | 71578 | 15: 34801602 | 71582 | 15: 34866384 | 71583 | 15: 34892433 |
| 71588 | 15: 34974291 | 71591 | 15: 35040731 | 71592 | 15: 35049691 | 71598 | 15: 35277152 | 71599 | 15: 35337946 |
| 71604 | 15: 35461378 | 71611 | 15: 35646977 | 71612 | 15: 35648952 | 71615 | 15: 35707211 | 71617 | 15: 35715226 |
| 71635 | 15: 36132660 | 71636 | 15: 36169165 | 71639 | 15: 36183126 | 71640 | 15: 36219316 | 71645 | 15: 36321976 |
| 71655 | 15: 36576380 | 71657 | 15: 36603077 | 71678 | 15: 37088135 | 71684 | 15: 37186172 | 71706 | 15: 37547916 |
| 71731 | 15: 38265442 | 71738 | 15: 38472609 | 71769 | 15: 39119041 | 71776 | 15: 39333095 | 71777 | 15: 39347738 |
| 71781 | 15: 39412550 | 71785 | 15: 39545268 | 71799 | 15: 39871286 | 71804 | 15: 39927546 | 71805 | 15: 39927550 |
| 71806 | 15: 39927601 | 71809 | 15: 40067276 | 71812 | 15: 40162512 | 71817 | 15: 40279882 | 71818 | 15: 40279893 |
| 71819 | 15: 40279894 | 71820 | 15: 40280012 | 71823 | 15: 40449461 | 71824 | 15: 40449484 | 71826 | 15: 40502987 |
| 71832 | 15: 40632071 | 71833 | 15: 40641136 | 71835 | 15: 40723287 | 71837 | 15: 40759558 | 71838 | 15: 40847130 |
| 71845 | 15: 40955841 | 71849 | 15: 41014898 | 71850 | 15: 41016306 | 71852 | 15: 41150753 | 71860 | 15: 41290689 |
| 71869 | 15: 41440324 | 71877 | 15: 41698377 | 71878 | 15: 41698492 | 71879 | 15: 41698511 | 71880 | 15: 41705974 |
| 71881 | 15: 41705975 | 71882 | 15: 41706097 | 71885 | 15: 41765753 | 71886 | 15: 41767083 | 71887 | 15: 41768163 |
| 71889 | 15: 41814640 | 71901 | 15: 42032808 | 71904 | 15: 42124927 | 71907 | 15: 42185672 | 71909 | 15: 42231274 |
| 71912 | 15: 42264995 | 71924 | 15: 42509261 | 71926 | 15: 42590389 | 71932 | 15: 42752352 | 71934 | 15: 42787098 |
| 71936 | 15: 42818617 | 71939 | 15: 42848256 | 71940 | 15: 42854523 | 71951 | 15: 43042213 | 71952 | 15: 43045041 |
| 71953 | 15: 43046535 | 71954 | 15: 43049707 | 71955 | 15: 43053427 | 71956 | 15: 43055412 | 71957 | 15: 43056476 |
| 71958 | 15: 43057569 | 71959 | 15: 43058822 | 71960 | 15: 43059592 | 71962 | 15: 43063078 | 71963 | 15: 43063710 |
| 71964 | 15: 43097237 | 71965 | 15: 43098155 | 71970 | 15: 43259917 | 71971 | 15: 43260963 | 71972 | 15: 43262025 |
| 71973 | 15: 43262890 | 71974 | 15: 43269560 | 71975 | 15: 43276395 | 71976 | 15: 43286382 | 71977 | 15: 43288762 |
| 71978 | 15: 43292368 | 71979 | 15: 43293082 | 71980 | 15: 43294086 | 71989 | 15: 43476234 | 71993 | 15: 43560696 |
| 71994 | 15: 43561701 | 71995 | 15: 43563456 | 71996 | 15: 43566385 | 71997 | 15: 43573222 | 71998 | 15: 43580011 |
| 72000 | 15: 43625295 | 72002 | 15: 43642580 | 72003 | 15: 43664227 | 72007 | 15: 43741512 | 72020 | 15: 43936008 |
| 72022 | 15: 43939348 | 72023 | 15: 43939353 | 72024 | 15: 43939488 | 72046 | 15: 44460580 | 72047 | 15: 44479177 |
| 72056 | 15: 44643217 | 72058 | 15: 44735861 | 72075 | 15: 45129015 | 72083 | 15: 45210490 | 72084 | 15: 45221891 |
| 72085 | 15: 45246954 | 72088 | 15: 45346073 | 72090 | 15: 45379221 | 72091 | 15: 45415112 | 72103 | 15: 45716064 |
| 72104 | 15: 45722551 | 72109 | 15: 45827220 | 72126 | 15: 46426137 | 72128 | 15: 46464105 | 72131 | 15: 46490284 |
| 72133 | 15: 46492960 | 72134 | 15: 46494185 | 72135 | 15: 46500386 | 72136 | 15: 46500459 | 72137 | 15: 46500629 |
| 72138 | 15: 46500752 | 72139 | 15: 46505669 | 72140 | 15: 46506092 | 72141 | 15: 46506887 | 72142 | 15: 46506889 |
| 72143 | 15: 46506939 | 72144 | 15: 46507314 | 72145 | 15: 46507929 | 72146 | 15: 46508001 | 72147 | 15: 46508021 |
| 72148 | 15: 46512024 | 72149 | 15: 46512113 | 72156 | 15: 46613300 | 72159 | 15: 46636679 | 72161 | 15: 46645269 |
| 72162 | 15: 46646092 | 72173 | 15: 46864410 | 72174 | 15: 46866871 | 72178 | 15: 46967802 | 72189 | 15: 47187912 |
| 72190 | 15: 47188138 | 72191 | 15: 47188507 | 72201 | 15: 47615122 | 72202 | 15: 47641029 | 72205 | 15: 47779635 |
| 72206 | 15: 47779640 | 72207 | 15: 47779664 | 72208 | 15: 47779674 | 72209 | 15: 47787011 | 72210 | 15: 47787118 |
| 72211 | 15: 47848072 | 72212 | 15: 47856278 | 72213 | 15: 47857368 | 72215 | 15: 47862235 | 72216 | 15: 47862345 |
| 72217 | 15: 47867091 | 72222 | 15: 48008735 | 72224 | 15: 48040175 | 72225 | 15: 48040515 | 72226 | 15: 48040954 |
| 72228 | 15: 48078030 | 72229 | 15: 48078755 | 72230 | 15: 48102626 | 72231 | 15: 48102703 | 72232 | 15: 48103271 |
| 72233 | 15: 48103402 | 72250 | 15: 48373937 | 72251 | 15: 48378524 | 72252 | 15: 48386761 | 72253 | 15: 48390883 |
| 72254 | 15: 48396110 | 72255 | 15: 48397484 | 72266 | 15: 48867306 | 72267 | 15: 48952912 | 72275 | 15: 49295120 |
| 72287 | 15: 49686635 | 72289 | 15: 49798310 | 72292 | 15: 49812025 | 72296 | 15: 50073269 | 72302 | 15: 50348347 |
| 72305 | 15: 50405124 | 72306 | 15: 50418732 | 72311 | 15: 50513682 | 72312 | 15: 50513924 | 72326 | 15: 51057213 |
| 72329 | 15: 51193044 | 72332 | 15: 51271630 | 72333 | 15: 51275342 | 72336 | 15: 51360447 | 72337 | 15: 51360592 |
| 72338 | 15: 51362026 | 72339 | 15: 51362043 | 72340 | 15: 51362061 | 72341 | 15: 51362080 | 72342 | 15: 51362276 |
| 72343 | 15: 51362277 | 72344 | 15: 51362431 | 72345 | 15: 51362457 | 72346 | 15: 51362458 | 72347 | 15: 51364116 |
| 72348 | 15: 51364137 | 72349 | 15: 51420115 | 72350 | 15: 51441064 | 72351 | 15: 51441095 | 72352 | 15: 51511445 |
| 72354 | 15: 51518000 | 72355 | 15: 51519061 | 72357 | 15: 51521753 | 72358 | 15: 51523084 | 72359 | 15: 51529172 |
| 72360 | 15: 51530078 | 72361 | 15: 51535001 | 72362 | 15: 51542928 | 72366 | 15: 51558105 | 72367 | 15: 51563195 |
| 72370 | 15: 51568569 | 72371 | 15: 51576564 | 72372 | 15: 51610810 | 72376 | 15: 51684740 | 72377 | 15: 51687709 |
| 72379 | 15: 51689738 | 72382 | 15: 51736976 | 72384 | 15: 51790467 | 72386 | 15: 51803577 | 72394 | 15: 51988641 |
| 72397 | 15: 52007899 | 72404 | 15: 52185081 | 72406 | 15: 52200879 | 72413 | 15: 52360124 | 72424 | 15: 52658762 |
| 72425 | 15: 52660106 | 72426 | 15: 52673691 | 72441 | 15: 53067270 | 72445 | 15: 53137213 | 72449 | 15: 53308023 |
| 72451 | 15: 53309880 | 72452 | 15: 53310424 | 72453 | 15: 53311034 | 72454 | 15: 53311897 | 72455 | 15: 53312462 |
| 72456 | 15: 53314094 | 72457 | 15: 53314682 | 72458 | 15: 53316670 | 72459 | 15: 53319540 | 72460 | 15: 53320533 |
| 72461 | 15: 53321307 | 72462 | 15: 53323633 | 72463 | 15: 53324367 | 72465 | 15: 53325750 | 72466 | 15: 53326273 |
| 72467 | 15: 53326849 | 72468 | 15: 53327898 | 72469 | 15: 53332122 | 72471 | 15: 53333616 | 72472 | 15: 53338788 |
| 72476 | 15: 53398368 | 72478 | 15: 53441398 | 72486 | 15: 53709788 | 7248 | 15: 53727634 | 72488 | 15: 53729356 |
| 72489 | 15: 53738936 | 72490 | 15: 53743609 | 72491 | 15: 53753517 | 72492 | 15: 53761189 | 72494 | 15: 53788777 |
| 72495 | 15: 53796595 | 72496 | 15: 53800970 | 72502 | 15: 53945334 | 72518 | 15: 54255643 | 72527 | 15: 54462755 |
| 72530 | 15: 54489427 | 72531 | 15: 54500400 | 72541 | 15: 54748501 | 72544 | 15: 54834057 | 72548 | 15: 54885008 |
| 72549 | 15: 54885114 | 72550 | 15: 54885117 | 72551 | 15: 54889719 | 72552 | 15: 54889820 | 72556 | 15: 55119302 |
| 72565 | 15: 55289790 | 72569 | 15: 55444251 | 72574 | 15: 55529280 | 72582 | 15: 55734507 | 72589 | 15: 56022102 |
| 72596 | 15: 56200579 | 72603 | 15: 56497282 | 72604 | 15: 56497450 | 72605 | 15: 56501007 | 72607 | 15: 56515635 |
| 72608 | 15: 56515639 | 72609 | 15: 56515642 | 72610 | 15: 56515714 | 72611 | 15: 56518845 | 72612 | 15: 56518860 |
| 72613 | 15: 56518880 | 72614 | 15: 56518892 | 72615 | 15: 56518948 | 72616 | 15: 56519916 | 72617 | 15: 56520003 |
| 72618 | 15: 56520009 | 72619 | 15: 56522354 | 72620 | 15: 56523085 | 72621 | 15: 56523145 | 72622 | 15: 56525703 |
| 72623 | 15: 56526321 | 72624 | 15: 56527735 | 72625 | 15: 56529668 | 72626 | 15: 56548649 | 72644 | 15: 56809212 |
| 72675 | 15: 57003970 | 72684 | 15: 57272924 | 72689 | 15: 57423300 | 72692 | 15: 57500020 | 72694 | 15: 57536234 |
| 72695 | 15: 57536250 | 72703 | 15: 57718142 | 72709 | 15: 57803493 | 72712 | 15: 57854603 | 72722 | 15: 58001428 |
| 72727 | 15: 58088362 | 72734 | 15: 58241375 | 72736 | 15: 58282071 | 72737 | 15: 58283033 | 72740 | 15: 58330257 |
| 72760 | 15: 58671427 | 72773 | 15: 59112131 | 72787 | 15: 59537850 | 72795 | 15: 59796390 | 72809 | 15: 60285268 |
| 72813 | 15: 60368718 | 72815 | 15: 60428854 | 72820 | 15: 60523980 | 72823 | 15: 60608578 | 72850 | 15: 61257843 |
| 72854 | 15: 61342496 | 72868 | 15: 61603248 | 72869 | 15: 61639615 | 72871 | 15: 61721710 | 72872 | 15: 61721767 |
| 72873 | 15: 61724284 | 72874 | 15: 61724369 | 72875 | 15: 61724392 | 72876 | 15: 61725702 | 72879 | 15: 61767375 |
| 72894 | 15: 62032399 | 72907 | 15: 62467385 | 72912 | 15: 62561316 | 72914 | 15: 62563939 | 72915 | 15: 62586048 |
| 72916 | 15: 62586078 | 72920 | 15: 62652293 | 72938 | 15: 63066650 | 72940 | 15: 63100087 | 72941 | 15: 63107739 |
| 72949 | 15: 63295827 | 72951 | 15: 63300815 | 72952 | 15: 63303147 | 72960 | 15: 63529465 | 72962 | 15: 63574887 |
| 72964 | 15: 63579300 | 72969 | 15: 63631630 | 72972 | 15: 63722937 | 72973 | 15: 63723070 | 72974 | 15: 63743524 |
| 72979 | 15: 63917098 | 72983 | 15: 63967865 | 72997 | 15: 64256628 | 72999 | 15: 64283258 | 73001 | 15: 64333153 |
| 73004 | 15: 64416446 | 73023 | 15: 64714146 | 73026 | 15: 64744225 | 73027 | 15: 64771598 | 73031 | 15: 64801402 |
| 73033 | 15: 64806923 | 73038 | 15: 64854504 | 73040 | 15: 64908292 | 73046 | 15: 64986216 | 73050 | 15: 65029608 |
| 73056 | 15: 65148971 | 73064 | 15: 65275286 | 73066 | 15: 65335638 | 73075 | 15: 65436899 | 73078 | 15: 65521232 |
| 73080 | 15: 65594811 | 73081 | 15: 65607689 | 73083 | 15: 65622127 | 73085 | 15: 65656084 | 73086 | 15: 65670390 |
| 73091 | 15: 65822176 | 73093 | 15: 65854403 | 73105 | 15: 66145675 | 73106 | 15: 66156197 | 73115 | 15: 66337078 |
| 73117 | 15: 66339107 | 73119 | 15: 66380166 | 73121 | 15: 66418487 | 73124 | 15: 66460251 | 73127 | 15: 66496173 |
| 73129 | 15: 66543651 | 73141 | 15: 66724079 | 73146 | 15: 66881298 | 73154 | 15: 67179102 | 73164 | 15: 67470477 |
| 73176 | 15: 67880702 | 73196 | 15: 68313970 | 73199 | 15: 68378183 | 73216 | 15: 68982890 | 73217 | 15: 69062551 |
| 73224 | 15: 69173709 | 73249 | 15: 70010700 | 73255 | 15: 70308624 | 73259 | 15: 70429364 | 73262 | 15: 70465643 |
| 73282 | 15: 71042092 | 73289 | 15: 71280393 | 73299 | 15: 71468201 | 73300 | 15: 71468245 | 73315 | 15: 71909279 |
| 73349 | 15: 72924654 | 73350 | 15: 72992522 | 73355 | 15: 73041310 | 73358 | 15: 73097589 | 73360 | 15: 73124963 |
| 73362 | 15: 73143263 | 73363 | 15: 73144008 | 73364 | 15: 73144009 | 73365 | 15: 73144095 | 73366 | 15: 73144112 |
| 73367 | 15: 73144117 | 73368 | 15: 73144142 | 73372 | 15: 73231049 | 73373 | 15: 73250550 | 73376 | 15: 73270606 |
| 73385 | 15: 73422749 | 73386 | 15: 73470922 | 73389 | 15: 73555524 | 73390 | 15: 73555544 | 73391 | 15: 73555546 |
| 73392 | 15: 73555549 | 73394 | 15: 73590486 | 73400 | 15: 73747012 | 73407 | 15: 74016858 | 73408 | 15: 74019876 |
| 73409 | 15: 74047524 | 73412 | 15: 74101712 | 73413 | 15: 74120008 | 73421 | 15: 74204147 | 73422 | 15: 74221807 |
| 73423 | 15: 74236733 | 73440 | 15: 74680300 | 73442 | 15: 74706685 | 73443 | 15: 74715602 | 73444 | 15: 74730746 |
| 73445 | 15: 74736252 | 73448 | 15: 74763309 | 73449 | 15: 74765962 | 73456 | 15: 74857238 | 73476 | 15: 75401306 |
| 73484 | 15: 75520621 | 73496 | 15: 75764247 | 73498 | 15: 75807751 | 73505 | 15: 76018207 | 73513 | 15: 76268285 |
| 73514 | 15: 76284024 | 73515 | 15: 76295386 | 73517 | 15: 76357310 | 73519 | 15: 76369909 | 73520 | 15: 76423356 |
| 73525 | 15: 76490114 | 73530 | 15: 76627029 | 73531 | 15: 76628527 | 73533 | 15: 76676572 | 73542 | 15: 76909234 |
| 73543 | 15: 76913709 | 73554 | 15: 77213130 | 73558 | 15: 77256270 | 73561 | 15: 77333647 | 73563 | 15: 77341921 |
| 73569 | 15: 77457892 | 73570 | 15: 77533291 | 73572 | 15: 77565837 | 73573 | 15: 77581726 | 73574 | 15: 77581747 |
| 73575 | 15: 77581752 | 73576 | 15: 77581872 | 73577 | 15: 77581882 | 73580 | 15: 77638946 | 73582 | 15: 77676086 |
| 73590 | 15: 77841257 | 73591 | 15: 77844974 | 73597 | 15: 77988330 | 73598 | 15: 77989577 | 73600 | 15: 77997353 |
| 73601 | 15: 78001161 | 73603 | 15: 78001840 | 73604 | 15: 78003499 | 73605 | 15: 78004209 | 73606 | 15: 78008228 |
| 73607 | 15: 78010723 | 73608 | 15: 78011919 | 73610 | 15: 78017138 | 73613 | 15: 78027580 | 73614 | 15: 78028904 |
| 73615 | 15: 78032066 | 73616 | 15: 78033166 | 73617 | 15: 78033926 | 73618 | 15: 78036014 | 73619 | 15: 78037096 |
| 73620 | 15: 78043902 | 73621 | 15: 78049025 | 73623 | 15: 78065440 | 73624 | 15: 78069489 | 73637 | 15: 78497070 |
| 73643 | 15: 78705678 | 73644 | 15: 78711863 | 73654 | 15: 79013310 | 73662 | 15: 79316918 | 73663 | 15: 79316919 |
| 73665 | 15: 79446531 | 73666 | 15: 79447076 | 73667 | 15: 79447126 | 73668 | 15: 79447145 | 73671 | 15: 79651229 |
| 73672 | 15: 79651253 | 73684 | 15: 79994534 | 73699 | 15: 80313710 | 73702 | 15: 80329440 | 73707 | 15: 80387982 |
| 73711 | 15: 80447483 | 73714 | 15: 80528280 | 73722 | 15: 80779796 | 73724 | 15: 80825322 | 73725 | 15: 80846525 |
| 73728 | 15: 80988789 | 73733 | 15: 81116878 | 73735 | 15: 81144733 | 73736 | 15: 81149287 | 73737 | 15: 81161372 |
| 73739 | 15: 81168190 | 73744 | 15: 81197998 | 73751 | 15: 81267470 | 73752 | 15: 81279270 | 73759 | 15: 81372132 |
| 73775 | 15: 81857983 | 73793 | 15: 82380481 | 73799 | 15: 82629156 | 73806 | 15: 82720061 | 73807 | 15: 82720089 |
| 73810 | 15: 82745967 | 73811 | 15: 82746237 | 73815 | 15: 82788081 | 73817 | 15: 82797551 | 73819 | 15: 82843451 |
| 73820 | 15: 82851394 | 73824 | 15: 82933219 | 73825 | 15: 82949122 | 73830 | 15: 83006747 | 73831 | 15: 83013517 |
| 73832 | 15: 83013534 | 73840 | 15: 83073328 | 73848 | 15: 83277486 | 73849 | 15: 83288242 | 73850 | 15: 83309084 |
| 73855 | 15: 83458586 | 73865 | 15: 83567476 | 73866 | 15: 83583523 | 73867 | 15: 83586864 | 73868 | 15: 83586930 |
| 73911 | 16: 752827 | 73914 | 16: 766023 | 73915 | 16: 804997 | 73916 | 16: 806537 | 73922 | 16: 921175 |
| 73929 | 16: 978613 | 73937 | 16: 1181761 | 73939 | 16: 1204788 | 73941 | 16: 1214929 | 73946 | 16: 1324936 |
| 73963 | 16: 1582068 | 73964 | 16: 1599816 | 73965 | 16: 1625990 | 73988 | 16: 2055227 | 73992 | 16: 2194637 |
| 73993 | 16: 2224824 | 73995 | 16: 2248476 | 73996 | 16: 2263389 | 73997 | 16: 2277642 | 74004 | 16: 2402507 |
| 74005 | 16: 2467947 | 74013 | 16: 2602159 | 74017 | 16: 2743842 | 74023 | 16: 2938620 | 74026 | 16: 3018942 |
| 74030 | 16: 3067157 | 74038 | 16: 3201109 | 74040 | 16: 3201263 | 74042 | 16: 3253128 | 74043 | 16: 3269182 |
| 74044 | 16: 3269280 | 74045 | 16: 3270297 | 74051 | 16: 3378716 | 74055 | 16: 3405375 | 74059 | 16: 3465967 |
| 74062 | 16: 3607615 | 74065 | 16: 3643356 | 74066 | 16: 3644483 | 74073 | 16: 3791803 | 74080 | 16: 3992289 |
| 74087 | 16: 4106339 | 74088 | 16: 4109307 | 74093 | 16: 4171303 | 74099 | 16: 4262626 | 74100 | 16: 4262650 |
| 74101 | 16: 4265692 | 74103 | 16: 4290878 | 74109 | 16: 4423692 | 74114 | 16: 4475911 | 74119 | 16: 4592258 |
| 74122 | 16: 4640118 | 74125 | 16: 4717716 | 74129 | 16: 4872430 | 74130 | 16: 4898040 | 74133 | 16: 4999290 |
| 74140 | 16: 5116140 | 74146 | 16: 5243133 | 74147 | 16: 5265259 | 74148 | 16: 5265363 | 74162 | 16: 6052639 |
| 74164 | 16: 6060113 | 74166 | 16: 6207254 | 74168 | 16: 6308221 | 74169 | 16: 6317267 | 74170 | 16: 6318112 |
| 74171 | 16: 6319483 | 74172 | 16: 6323633 | 74173 | 16: 6325179 | 74175 | 16: 6332032 | 74176 | 16: 6333222 |
| 74177 | 16: 6335935 | 74180 | 16: 6376238 | 74191 | 16: 6749567 | 74204 | 16: 7268343 | 74214 | 16: 7503961 |
| 74270 | 16: 9734287 | 74280 | 16: 9996877 | 74307 | 16: 10894806 | 74318 | 16: 11281651 | 74336 | 16: 11756836 |
| 74343 | 16: 11874391 | 74347 | 16: 11985702 | 74364 | 16: 12483562 | 74365 | 16: 12486013 | 74378 | 16: 12756820 |
| 74383 | 16: 12870981 | 74400 | 16: 13072437 | 74411 | 16: 13341826 | 74415 | 16: 13479712 | 74431 | 16: 13921125 |
| 74441 | 16: 14274292 | 74444 | 16: 14375476 | 74450 | 16: 14512750 | 74456 | 16: 14612740 | 74457 | 16: 14612809 |
| 74485 | 16: 15352405 | 74486 | 16: 15355419 | 74514 | 16: 16201608 | 74519 | 16: 16433665 | 74520 | 16: 16433701 |
| 74532 | 16: 16902875 | 74586 | 16: 18664935 | 74596 | 16: 18957732 | 74624 | 16: 19622167 | 74629 | 16: 19786076 |
| 74645 | 16: 20175121 | 74649 | 16: 20294926 | 74659 | 16: 20540783 | 74660 | 16: 20555295 | 74674 | 16: 20905213 |
| 74676 | 16: 20943232 | 74715 | 16: 21954699 | 74722 | 16: 22188120 | 74724 | 16: 22316546 | 74726 | 16: 22390334 |
| 74729 | 16: 22420005 | 74742 | 16: 22743613 | 74744 | 16: 22775537 | 74745 | 16: 22786666 | 74762 | 16: 23286838 |
| 74768 | 16: 23427214 | 74769 | 16: 23439282 | 74770 | 16: 23508581 | 74771 | 16: 23518024 | 74772 | 16: 23544281 |
| 74773 | 16: 23557997 | 74776 | 16: 23621715 | 74782 | 16: 23717545 | 74783 | 16: 23721462 | 74792 | 16: 23915566 |
| 74793 | 16: 23918537 | 74801 | 16: 24174300 | 74802 | 16: 24198154 | 74806 | 16: 24253488 | 74809 | 16: 24321784 |
| 74822 | 16: 24770805 | 74844 | 16: 24996364 | 74847 | 16: 25007172 | 74856 | 16: 25057711 | 74879 | 16: 25209115 |
| 74900 | 16: 25434176 | 74908 | 16: 25723685 | 74909 | 16: 25753006 | 74910 | 16: 25760167 | 74911 | 16: 25845363 |
| 74915 | 16: 25999582 | 74916 | 16: 26009236 | 74917 | 16: 26012436 | 74918 | 16: 26021774 | 74920 | 16: 26036756 |
| 74921 | 16: 26037402 | 74924 | 16: 26139905 | 74925 | 16: 26150072 | 74926 | 16: 26150074 | 74927 | 16: 26153413 |
| 74929 | 16: 26186921 | 74930 | 16: 26216113 | 74931 | 16: 26217301 | 74933 | 16: 26220845 | 74934 | 16: 26227564 |
| 74935 | 16: 26274073 | 74936 | 16: 26296242 | 74940 | 16: 26352124 | 74941 | 16: 26356312 | 74943 | 16: 26451726 |
| 74951 | 16: 26637237 | 74973 | 16: 27186026 | 74989 | 16: 27756698 | 74996 | 16: 27880735 | 74997 | 16: 27880880 |
| 75002 | 16: 27952368 | 75003 | 16: 28008613 | 75004 | 16: 28008626 | 75013 | 16: 28274084 | 75024 | 16: 28665499 |
| 75034 | 16: 28945983 | 75036 | 16: 29003100 | 75057 | 16: 29692650 | 75059 | 16: 29709436 | 75060 | 16: 29760720 |
| 75061 | 16: 29760768 | 75064 | 16: 29872847 | 75065 | 16: 29893692 | 75079 | 16: 30287006 | 75081 | 16: 30348202 |
| 75088 | 16: 30604134 | 75092 | 16: 30729515 | 75096 | 16: 30772837 | 75098 | 16: 30794250 | 75099 | 16: 30795047 |
| 75100 | 16: 30802513 | 75102 | 16: 30839358 | 75108 | 16: 31046070 | 75109 | 16: 31054739 | 75120 | 16: 31360463 |
| 75130 | 16: 31587509 | 75131 | 16: 31613864 | 75137 | 16: 31734054 | 75146 | 16: 31961054 | 75147 | 16: 31961057 |
| 75151 | 16: 32067908 | 75157 | 16: 32356916 | 75162 | 16: 32428255 | 75165 | 16: 32550214 | 75180 | 16: 32851343 |
| 75183 | 16: 32875785 | 75184 | 16: 32880400 | 75187 | 16: 32951618 | 75188 | 16: 32951739 | 75215 | 16: 33649281 |
| 75216 | 16: 33650436 | 75220 | 16: 33885770 | 75224 | 16: 33965943 | 75226 | 16: 33984101 | 75247 | 16: 34572625 |
| 75250 | 16: 34620173 | 75254 | 16: 34722052 | 75257 | 16: 34852541 | 75262 | 16: 34933617 | 75263 | 16: 34933651 |
| 75267 | 16: 35001017 | 75270 | 16: 35137739 | 75272 | 16: 35194830 | 75273 | 16: 35194886 | 75274 | 16: 35194897 |
| 75276 | 16: 35202714 | 75277 | 16: 35234653 | 75278 | 16: 35244272 | 75279 | 16: 35254252 | 75280 | 16: 35290661 |
| 75281 | 16: 35295775 | 75295 | 16: 35792938 | 75302 | 16: 35995859 | 75303 | 16: 36000751 | 75304 | 16: 36004313 |
| 75307 | 16: 36081689 | 75313 | 16: 36290108 | 75314 | 16: 36299734 | 75317 | 16: 36326781 | 75321 | 16: 36365140 |
| 75330 | 16: 36566768 | 75335 | 16: 36697557 | 75336 | 16: 36712618 | 75337 | 16: 36775388 | 75344 | 16: 36945543 |
| 75345 | 16: 36969420 | 75365 | 16: 37448884 | 75366 | 16: 37448890 | 75376 | 16: 37796926 | 75381 | 16: 37913794 |
| 75382 | 16: 37913795 | 75383 | 16: 37913847 | 75384 | 16: 37913888 | 75386 | 16: 37937575 | 75397 | 16: 38107328 |
| 75399 | 16: 38122376 | 75401 | 16: 38137107 | 75402 | 16: 38150887 | 75406 | 16: 38234762 | 75407 | 16: 38244862 |
| 75423 | 16: 38532788 | 75431 | 16: 38885897 | 75458 | 16: 39922839 | 75459 | 16: 39929733 | 75482 | 16: 40598638 |
| 75485 | 16: 40704573 | 75500 | 16: 41266328 | 75513 | 16: 41835712 | 75514 | 16: 41852637 | 75515 | 16: 41881957 |
| 75516 | 16: 41882451 | 75517 | 16: 41882906 | 75518 | 16: 41882957 | 75520 | 16: 41883859 | 75521 | 16: 41884250 |
| 75522 | 16: 41889773 | 75523 | 16: 41890000 | 75524 | 16: 41892823 | 75525 | 16: 41896465 | 75526 | 16: 41898495 |
| 75527 | 16: 41898551 | 75528 | 16: 41908374 | 75529 | 16: 41908542 | 75530 | 16: 41909276 | 75531 | 16: 41909349 |
| 75532 | 16: 41909492 | 75533 | 16: 41909626 | 75534 | 16: 41909729 | 75535 | 16: 41909886 | 75536 | 16: 41929565 |
| 75537 | 16: 41931893 | 75538 | 16: 41931907 | 75539 | 16: 41934619 | 75540 | 16: 41934651 | 75541 | 16: 41934687 |
| 75543 | 16: 41947567 | 75544 | 16: 41964938 | 75554 | 16: 42253482 | 75557 | 16: 42465089 | 75558 | 16: 42547847 |
| 75559 | 16: 42600403 | 75569 | 16: 43061490 | 75575 | 16: 43278476 | 75576 | 16: 43330994 | 75578 | 16: 43398900 |
| 75583 | 16: 43745014 | 75593 | 16: 44139005 | 75604 | 16: 44403647 | 75606 | 16: 44524286 | 75609 | 16: 44660436 |
| 75610 | 16: 44759709 | 75625 | 16: 45375047 | 75641 | 16: 46123551 | 75647 | 16: 46250903 | 75649 | 16: 46275222 |
| 75650 | 16: 46286961 | 75651 | 16: 46287045 | 75658 | 16: 46502252 | 75665 | 16: 46683276 | 75671 | 16: 46787853 |
| 75672 | 16: 46830087 | 75680 | 16: 47040946 | 75682 | 16: 47083406 | 75684 | 16: 47094623 | 75685 | 16: 47120517 |
| 75689 | 16: 47196401 | 75697 | 16: 47379730 | 75698 | 16: 47379738 | 75702 | 16: 47529414 | 75709 | 16: 47649083 |
| 75710 | 16: 47653055 | 75711 | 16: 47658993 | 75719 | 16: 47876641 | 75748 | 16: 48471006 | 75749 | 16: 48471012 |
| 75750 | 16: 48471070 | 75767 | 16: 48815391 | 75788 | 16: 49290662 | 75798 | 16: 49608069 | 75800 | 16: 49640815 |
| 75804 | 16: 49736666 | 75816 | 16: 50023350 | 75856 | 16: 51315511 | 75862 | 16: 51572737 | 75866 | 16: 51638935 |
| 75870 | 16: 51814662 | 75871 | 16: 51818542 | 75872 | 16: 51823133 | 75873 | 16: 51827521 | 75874 | 16: 51828907 |
| 75875 | 16: 51830294 | 75876 | 16: 51830796 | 75877 | 16: 51841289 | 75879 | 16: 51854505 | 75881 | 16: 51899632 |
| 75889 | 16: 52062903 | 75892 | 16: 52124016 | 75896 | 16: 52189080 | 75897 | 16: 52203550 | 75905 | 16: 52391190 |
| 75906 | 16: 52391213 | 75907 | 16: 52401546 | 75908 | 16: 52413488 | 75911 | 16: 52492743 | 75920 | 16: 52664549 |
| 75922 | 16: 52715395 | 75933 | 16: 53089976 | 75945 | 16: 53432925 | 75951 | 16: 53742380 | 75952 | 16: 53742397 |
| 75953 | 16: 53742405 | 75954 | 16: 53742482 | 75960 | 16: 53807690 | 75962 | 16: 53819240 | 75971 | 16: 54022291 |
| 75972 | 16: 54037567 | 75973 | 16: 54047358 | 76003 | 16: 54893701 | 76004 | 16: 54916866 | 76005 | 16: 54917648 |
| 76008 | 16: 54974984 | 76009 | 16: 54975864 | 76010 | 16: 55000694 | 76011 | 16: 55015703 | 76012 | 16: 55021774 |
| 76013 | 16: 55109042 | 76032 | 16: 55624726 | 76041 | 16: 55875606 | 76053 | 16: 56173257 | 76055 | 16: 56193959 |
| 76056 | 16: 56206450 | 76066 | 16: 56367576 | 76067 | 16: 56372228 | 76069 | 16: 56383292 | 76072 | 16: 56444611 |
| 76077 | 16: 56546719 | 76080 | 16: 56571482 | 76081 | 16: 56571485 | 76082 | 16: 56571494 | 76089 | 16: 56837687 |
| 76096 | 16: 57122424 | 76100 | 16: 57326320 | 76101 | 16: 57329788 | 76113 | 16: 57586740 | 76114 | 16: 57608446 |
| 76126 | 16: 57873879 | 76129 | 16: 58011779 | 76130 | 16: 58012586 | 76146 | 16: 58359793 | 76156 | 16: 58464078 |
| 76158 | 16: 58524073 | 76175 | 16: 58904562 | 76176 | 16: 58904595 | 76199 | 16: 59323799 | 76201 | 16: 59330153 |
| 76211 | 16: 59665282 | 76212 | 16: 59689730 | 76213 | 16: 59689802 | 76222 | 16: 59946371 | 76231 | 16: 60201651 |
| 76233 | 16: 60224702 | 76234 | 16: 60224712 | 76236 | 16: 60251070 | 76237 | 16: 60253242 | 76238 | 16: 60254578 |
| 76240 | 16: 60311442 | 76241 | 16: 60322616 | 76242 | 16: 60322634 | 76245 | 16: 60357272 | 76247 | 16: 60391026 |
| 76251 | 16: 60558830 | 76258 | 16: 60784981 | 76274 | 16: 61120937 | 76279 | 16: 61322631 | 76285 | 16: 61383011 |
| 76286 | 16: 61387337 | 76301 | 16: 61731673 | 76306 | 16: 61957053 | 76312 | 16: 62071218 | 76313 | 16: 62072068 |
| 76314 | 16: 62072151 | 76315 | 16: 62072468 | 76316 | 16: 62077343 | 76318 | 16: 62094667 | 76319 | 16: 62094728 |
| 76327 | 16: 62245438 | 76328 | 16: 62267314 | 76329 | 16: 62275255 | 76335 | 16: 62455462 | 76343 | 16: 62633633 |
| 76348 | 16: 62852449 | 76349 | 16: 62856876 | 76354 | 16: 62916406 | 76356 | 16: 62926757 | 76369 | 16: 63226438 |
| 76370 | 16: 63226475 | 76371 | 16: 63252161 | 76372 | 16: 63264840 | 76374 | 16: 63283115 | 76376 | 16: 63353590 |
| 76382 | 16: 63498489 | 76389 | 16: 63665761 | 76391 | 16: 63691003 | 76395 | 16: 63806226 | 76397 | 16: 63832124 |
| 76403 | 16: 63910273 | 76404 | 16: 63918137 | 76406 | 16: 63985213 | 76413 | 16: 64162147 | 76421 | 16: 64386831 |
| 76426 | 16: 64477575 | 76427 | 16: 64478870 | 76428 | 16: 64480600 | 76429 | 16: 64482426 | 76430 | 16: 64483727 |
| 76431 | 16: 64486623 | 76432 | 16: 64488080 | 76434 | 16: 64560495 | 76437 | 16: 64585141 | 76438 | 16: 64587028 |
| 76439 | 16: 64599607 | 76447 | 16: 64716072 | 76457 | 16: 64911089 | 76458 | 16: 64915512 | 76459 | 16: 64917512 |
| 76460 | 16: 64917539 | 76461 | 16: 64920273 | 76462 | 16: 64924645 | 76463 | 16: 64945487 | 76464 | 16: 64947395 |
| 76466 | 16: 64979912 | 76467 | 16: 64979914 | 76468 | 16: 64979920 | 76469 | 16: 64980083 | 76473 | 16: 65100643 |
| 76479 | 16: 65256823 | 76480 | 16: 65263949 | 76483 | 16: 65320518 | 76490 | 16: 65492703 | 76491 | 16: 65500984 |
| 76492 | 16: 65508159 | 76493 | 16: 65511465 | 76494 | 16: 65514812 | 76495 | 16: 65518974 | 76496 | 16: 65521306 |
| 76505 | 16: 65841167 | 76512 | 16: 66020681 | 76515 | 16: 66100353 | 76517 | 16: 66121444 | 76521 | 16: 66169638 |
| 76523 | 16: 66271884 | 76532 | 16: 66520379 | 76533 | 16: 66528712 | 76536 | 16: 66553762 | 76538 | 16: 66566651 |
| 76550 | 16: 66804665 | 76551 | 16: 66825677 | 76553 | 16: 66859242 | 76555 | 16: 66896436 | 76557 | 16: 66919773 |
| 76558 | 16: 66919950 | 76564 | 16: 67050756 | 76565 | 16: 67108343 | 76571 | 16: 67259430 | 76573 | 16: 67296635 |
| 76580 | 16: 67413442 | 76581 | 16: 67414503 | 76582 | 16: 67414576 | 76583 | 16: 67414591 | 76584 | 16: 67418245 |
| 76588 | 16: 67495932 | 76599 | 16: 67610069 | 76610 | 16: 67976663 | 76611 | 16: 67981588 | 76626 | 16: 68435498 |
| 76627 | 16: 68435550 | 76636 | 16: 68624997 | 76652 | 16: 69066498 | 76653 | 16: 69066625 | 76656 | 16: 69080402 |
| 76657 | 16: 69086868 | 76660 | 16: 69129862 | 76665 | 16: 69230127 | 76671 | 16: 69350387 | 76679 | 16: 69497807 |
| 76680 | 16: 69501212 | 76681 | 16: 69541904 | 76685 | 16: 69607606 | 76688 | 16: 69676831 | 76689 | 16: 69704477 |
| 76692 | 16: 69802000 | 76719 | 16: 70380777 | 76720 | 16: 70381368 | 76730 | 16: 70587239 | 76731 | 16: 70590791 |
| 76732 | 16: 70590919 | 76733 | 16: 70608188 | 76744 | 16: 70871474 | 76754 | 16: 71132906 | 76759 | 16: 71385575 |
| 76762 | 16: 71432921 | 76766 | 16: 71483613 | 76771 | 16: 71716120 | 76772 | 16: 71732153 | 76777 | 16: 71834818 |
| 76784 | 16: 71964464 | 76785 | 16: 71974676 | 76792 | 16: 72212208 | 76797 | 16: 72370528 | 76798 | 16: 72370550 |
| 76799 | 16: 72370562 | 76800 | 16: 72370636 | 76803 | 16: 72382117 | 76804 | 16: 72382163 | 76805 | 16: 72382236 |
| 76806 | 16: 72382280 | 76808 | 16: 72394792 | 76811 | 16: 72421917 | 76813 | 16: 72440326 | 76814 | 16: 72473155 |
| 76815 | 16: 72473197 | 76821 | 16: 72619958 | 76823 | 16: 72642068 | 76826 | 16: 72684703 | 76828 | 16: 72684778 |
| 76829 | 16: 72684811 | 76831 | 16: 72698852 | 76832 | 16: 72700502 | 76833 | 16: 72701289 | 76848 | 16: 73201335 |
| 76853 | 16: 73303554 | 76860 | 16: 73466388 | 76862 | 16: 73504205 | 76865 | 16: 73518067 | 76866 | 16: 73534249 |
| 76868 | 16: 73563070 | 76869 | 16: 73563134 | 76871 | 16: 73583302 | 76873 | 16: 73591363 | 76874 | 16: 73597712 |
| 76876 | 16: 73599978 | 76877 | 16: 73607828 | 76878 | 16: 73622925 | 76879 | 16: 73694168 | 76880 | 16: 73694195 |
| 76881 | 16: 73694803 | 76882 | 16: 73697167 | 76883 | 16: 73697204 | 76884 | 16: 73697206 | 76885 | 16: 73697258 |
| 76886 | 16: 73697700 | 76887 | 16: 73699147 | 76889 | 16: 73715301 | 76892 | 16: 73750079 | 76909 | 16: 74262736 |
| 76910 | 16: 74267964 | 76924 | 16: 74548368 | 76931 | 16: 74775935 | 76936 | 16: 74853499 | 76945 | 16: 75005400 |
| 76946 | 16: 75005468 | 76948 | 16: 75032047 | 76953 | 16: 75118546 | 76954 | 16: 75132927 | 76955 | 16: 75142495 |
| 76956 | 16: 75143544 | 76957 | 16: 75153428 | 76959 | 16: 75202467 | 76960 | 16: 75217027 | 76963 | 16: 75248051 |
| 76965 | 16: 75255731 | 76966 | 16: 75256528 | 76967 | 16: 75273493 | 76971 | 16: 75346062 | 76978 | 16: 75476985 |
| 76979 | 16: 75477188 | 76980 | 16: 75487044 | 76986 | 16: 75656899 | 76991 | 16: 75736008 | 76996 | 16: 75817486 |
| 77008 | 16: 76064461 | 77010 | 16: 76082386 | 77016 | 16: 76264493 | 77020 | 16: 76316949 | 77023 | 16: 76382298 |
| 77025 | 16: 76417156 | 77026 | 16: 76424214 | 77029 | 16: 76490659 | 77034 | 16: 76550668 | 77035 | 16: 76561525 |
| 77038 | 16: 76632577 | 77039 | 16: 76645921 | 77044 | 16: 76724147 | 77047 | 16: 76749641 | 77049 | 16: 76810446 |
| 77050 | 16: 76898157 | 77053 | 16: 77026179 | 77066 | 16: 77196110 | 77075 | 16: 77345710 | 77076 | 16: 77345711 |
| 77086 | 16: 77672463 | 77088 | 16: 77700431 | 77133 | 16: 78730933 | 77136 | 16: 78794726 | 77146 | 16: 78936706 |
| 77147 | 16: 78936848 | 77148 | 16: 78936900 | 77162 | 16: 79072375 | 77170 | 16: 79207667 | 77171 | 16: 79214144 |
| 77172 | 16: 79214863 | 77173 | 16: 79214898 | 77174 | 16: 79216805 | 77175 | 16: 79216824 | 77176 | 16: 79216879 |
| 77177 | 16: 79217281 | 77178 | 16: 79246839 | 77179 | 16: 79247022 | 77180 | 16: 79248848 | 77181 | 16: 79252434 |
| 77182 | 16: 79252455 | 77183 | 16: 79253093 | 77184 | 16: 79253212 | 77185 | 16: 79253954 | 77186 | 16: 79256053 |
| 77187 | 16: 79258337 | 77188 | 16: 79258375 | 77189 | 16: 79259607 | 77190 | 16: 79259608 | 77191 | 16: 79259624 |
| 77192 | 16: 79259664 | 77193 | 16: 79259693 | 77194 | 16: 79264907 | 77195 | 16: 79264964 | 77196 | 16: 79265514 |
| 77197 | 16: 79265600 | 77199 | 16: 79316462 | 77203 | 16: 79347181 | 77206 | 16: 79436693 | 77207 | 16: 79449942 |
| 77208 | 16: 79458180 | 77211 | 16: 79513573 | 77212 | 16: 79514562 | 77213 | 16: 79526906 | 77215 | 16: 79554621 |
| 77216 | 16: 79563778 | 77217 | 16: 79626644 | 77219 | 16: 79659075 | 77223 | 16: 79862681 | 77224 | 16: 79881468 |
| 77225 | 16: 79889426 | 77228 | 16: 80019106 | 77231 | 16: 80220495 | 77234 | 16: 80431031 | 77240 | 16: 80869601 |
| 77259 | 17: 425858 | 77280 | 17: 1125457 | 77282 | 17: 1162137 | 77283 | 17: 1162139 | 77284 | 17: 1162184 |
| 77285 | 17: 1162199 | 77286 | 17: 1162200 | 77300 | 17: 1367292 | 77306 | 17: 1539162 | 77316 | 17: 1782217 |
| 77338 | 17: 2449957 | 77357 | 17: 2912361 | 77367 | 17: 3145731 | 77376 | 17: 3382377 | 77391 | 17: 3546676 |
| 77399 | 17: 3801647 | 77403 | 17: 3941038 | 77406 | 17: 3948766 | 77407 | 17: 3948856 | 77408 | 17: 3950488 |
| 77409 | 17: 3950503 | 77410 | 17: 3979701 | 77413 | 17: 4046782 | 77419 | 17: 4192418 | 77424 | 17: 4272755 |
| 77426 | 17: 4298012 | 77432 | 17: 4394112 | 77442 | 17: 4571004 | 77447 | 17: 4633530 | 77448 | 17: 4657147 |
| 77453 | 17: 4721455 | 77454 | 17: 4721515 | 77456 | 17: 4770586 | 77460 | 17: 4835673 | 77465 | 17: 4973219 |
| 77479 | 17: 5317539 | 77481 | 17: 5375833 | 77482 | 17: 5398061 | 77490 | 17: 5679415 | 77491 | 17: 5679423 |
| 77492 | 17: 5679424 | 77493 | 17: 5679464 | 77494 | 17: 5679532 | 77496 | 17: 5728456 | 77512 | 17: 6078923 |
| 77513 | 17: 6107701 | 77516 | 17: 6161274 | 77517 | 17: 6198963 | 77518 | 17: 6205937 | 77520 | 17: 6220260 |
| 77521 | 17: 6229560 | 77522 | 17: 6258944 | 77526 | 17: 6341387 | 77532 | 17: 6459287 | 77533 | 17: 6491029 |
| 77537 | 17: 6552131 | 77539 | 17: 6614404 | 77540 | 17: 6625470 | 77541 | 17: 6626902 | 77544 | 17: 6698968 |
| 77545 | 17: 6701688 | 77546 | 17: 6707615 | 77547 | 17: 6710298 | 77548 | 17: 6715516 | 77554 | 17: 6811535 |
| 77555 | 17: 6819346 | 77559 | 17: 6940926 | 77560 | 17: 6984368 | 77561 | 17: 6985910 | 77563 | 17: 7027194 |
| 77588 | 17: 7507100 | 77596 | 17: 7694458 | 77598 | 17: 7697113 | 77602 | 17: 7770098 | 77612 | 17: 8012088 |
| 77613 | 17: 8012177 | 77621 | 17: 8235574 | 77637 | 17: 8588779 | 77661 | 17: 9138287 | 77674 | 17: 9441496 |
| 77683 | 17: 9768434 | 77684 | 17: 9768476 | 77688 | 17: 9888649 | 77695 | 17: 9978114 | 77696 | 17: 10024882 |
| 77698 | 17: 10050562 | 77700 | 17: 10077655 | 77702 | 17: 10100298 | 77705 | 17: 10110670 | 77709 | 17: 10169816 |
| 77710 | 17: 10176209 | 77717 | 17: 10353846 | 77720 | 17: 10489157 | 77730 | 17: 10813567 | 77731 | 17: 10814317 |
| 77749 | 17: 11330319 | 77765 | 17: 11778925 | 77771 | 17: 11817119 | 77780 | 17: 12090766 | 77797 | 17: 12362004 |
| 77814 | 17: 12766769 | 77823 | 17: 12938879 | 77828 | 17: 13174673 | 77832 | 17: 13361331 | 77837 | 17: 13490837 |
| 77838 | 17: 13490864 | 77849 | 17: 13820980 | 77850 | 17: 13883525 | 77853 | 17: 13941930 | 77865 | 17: 14194666 |
| 77866 | 17: 14208519 | 77867 | 17: 14212322 | 77868 | 17: 14214868 | 77870 | 17: 14226993 | 77871 | 17: 14233961 |
| 77874 | 17: 14255518 | 77875 | 17: 14257411 | 77876 | 17: 14258646 | 77877 | 17: 14265993 | 77878 | 17: 14267278 |
| 77879 | 17: 14268573 | 77885 | 17: 14447098 | 77889 | 17: 14526830 | 77892 | 17: 14548325 | 77897 | 17: 14641198 |
| 77912 | 17: 14905289 | 77913 | 17: 14921802 | 77920 | 17: 15088817 | 77925 | 17: 15159023 | 77929 | 17: 15328788 |
| 77931 | 17: 15328888 | 77937 | 17: 15455174 | 77940 | 17: 15527126 | 77948 | 17: 15652225 | 77952 | 17: 15705665 |
| 77959 | 17: 15911943 | 77967 | 17: 16144638 | 77970 | 17: 16239054 | 77981 | 17: 16560132 | 77983 | 17: 16659063 |
| 77984 | 17: 16684179 | 77989 | 17: 16808806 | 77990 | 17: 16833027 | 78001 | 17: 17127455 | 78002 | 17: 17127599 |
| 78004 | 17: 17132830 | 78007 | 17: 17224074 | 78021 | 17: 17389673 | 78028 | 17: 17475047 | 78029 | 17: 17475831 |
| 78048 | 17: 17963586 | 78050 | 17: 17992691 | 78054 | 17: 18028087 | 78057 | 17: 18062413 | 78060 | 17: 18161738 |
| 78064 | 17: 18284618 | 78087 | 17: 18873882 | 78092 | 17: 18900848 | 78094 | 17: 18961495 | 78102 | 17: 19217997 |
| 78130 | 17: 19581434 | 78135 | 17: 19786181 | 78141 | 17: 19961480 | 78142 | 17: 19965877 | 78144 | 17: 20018582 |
| 78149 | 17: 20111214 | 78152 | 17: 20217310 | 78157 | 17: 20333021 | 78227 | 17: 22641428 | 78231 | 17: 22853179 |
| 78245 | 17: 23369245 | 78266 | 17: 24189137 | 78275 | 17: 24523000 | 78285 | 17: 24792731 | 78294 | 17: 25041571 |
| 78312 | 17: 25581271 | 78323 | 17: 25977654 | 78324 | 17: 25977693 | 78325 | 17: 25977723 | 78326 | 17: 25977762 |
| 78369 | 17: 26883751 | 78370 | 17: 26915514 | 78393 | 17: 27503090 | 78394 | 17: 27559967 | 78399 | 17: 27653769 |
| 78408 | 17: 27921810 | 78413 | 17: 28059892 | 78427 | 17: 28334342 | 78428 | 17: 28334349 | 78439 | 17: 28702389 |
| 78464 | 17: 29480103 | 78469 | 17: 29662529 | 78478 | 17: 29993789 | 78489 | 17: 30232347 | 78490 | 17: 30304435 |
| 78497 | 17: 30487913 | 78510 | 17: 30674204 | 78511 | 17: 30696794 | 78512 | 17: 30709277 | 78516 | 17: 30799626 |
| 78524 | 17: 30996169 | 78544 | 17: 31562261 | 78549 | 17: 31735789 | 78551 | 17: 31735957 | 78561 | 17: 31998920 |
| 78562 | 17: 32030137 | 78571 | 17: 32394529 | 78572 | 17: 32394568 | 78573 | 17: 32403823 | 78574 | 17: 32403842 |
| 78594 | 17: 32777788 | 78599 | 17: 32874912 | 78618 | 17: 33384490 | 78641 | 17: 33842930 | 78655 | 17: 34228544 |
| 78665 | 17: 34544058 | 78678 | 17: 34902455 | 78685 | 17: 35149936 | 78686 | 17: 35186932 | 78694 | 17: 35488740 |
| 78704 | 17: 35746205 | 78706 | 17: 35787842 | 78707 | 17: 35799086 | 78709 | 17: 35857580 | 78712 | 17: 35940925 |
| 78719 | 17: 36144107 | 78728 | 17: 36520470 | 78737 | 17: 36882303 | 78781 | 17: 38220984 | 78810 | 17: 39276967 |
| 78812 | 17: 39326538 | 78837 | 17: 39843444 | 78842 | 17: 39882804 | 78852 | 17: 39935546 | 78860 | 17: 40113609 |
| 78865 | 17: 40279234 | 78867 | 17: 40310128 | 78868 | 17: 40314686 | 78879 | 17: 40566975 | 78883 | 17: 40575300 |
| 78886 | 17: 40597293 | 78894 | 17: 40765558 | 78897 | 17: 40880376 | 78898 | 17: 40882503 | 78905 | 17: 41002973 |
| 78932 | 17: 41864826 | 78941 | 17: 42170894 | 78942 | 17: 42186823 | 78943 | 17: 42188310 | 78946 | 17: 42229273 |
| 78954 | 17: 42393090 | 78956 | 17: 42415227 | 78970 | 17: 42715046 | 78976 | 17: 42768963 | 78978 | 17: 42821182 |
| 78982 | 17: 42891704 | 78983 | 17: 42923614 | 78997 | 17: 43357874 | 79000 | 17: 43436343 | 79003 | 17: 43553874 |
| 79006 | 17: 43577102 | 79007 | 17: 43577588 | 79008 | 17: 43594706 | 79011 | 17: 43609022 | 79012 | 17: 43623722 |
| 79029 | 17: 44194890 | 79032 | 17: 44224761 | 79040 | 17: 44531668 | 79046 | 17: 44677660 | 79052 | 17: 44819760 |
| 79058 | 17: 44927880 | 79063 | 17: 45086949 | 79064 | 17: 45091135 | 79066 | 17: 45172949 | 79068 | 17: 45178502 |
| 79069 | 17: 45178540 | 79070 | 17: 45180519 | 79071 | 17: 45180612 | 79072 | 17: 45181370 | 79073 | 17: 45182722 |
| 79091 | 17: 45616057 | 79094 | 17: 45734727 | 79112 | 17: 46168639 | 79113 | 17: 46171496 | 79127 | 17: 46408391 |
| 79135 | 17: 46615898 | 79136 | 17: 46624459 | 79142 | 17: 46676910 | 79187 | 17: 47856534 | 79215 | 17: 48637182 |
| 79244 | 17: 49321412 | 79268 | 17: 49848060 | 79273 | 17: 49923094 | 79285 | 17: 50262075 | 79287 | 17: 50270777 |
| 79288 | 17: 50274476 | 79315 | 17: 50731825 | 79316 | 17: 50769132 | 79319 | 17: 50841533 | 79320 | 17: 50864647 |
| 79322 | 17: 50902320 | 79326 | 17: 50955494 | 79327 | 17: 50959261 | 79339 | 17: 51345327 | 79342 | 17: 51469617 |
| 79343 | 17: 51470914 | 79346 | 17: 51529156 | 79348 | 17: 51546917 | 79355 | 17: 51627470 | 79359 | 17: 51685973 |
| 79360 | 17: 51689778 | 79364 | 17: 51762211 | 79366 | 17: 51792442 | 79368 | 17: 51813934 | 79375 | 17: 51958814 |
| 79377 | 17: 51959782 | 79379 | 17: 52027381 | 79395 | 17: 52307367 | 79403 | 17: 52573801 | 79414 | 17: 52988658 |
| 79417 | 17: 53020835 | 79423 | 17: 53275351 | 79428 | 17: 53388401 | 79430 | 17: 53390888 | 79431 | 17: 53448341 |
| 79433 | 17: 53470903 | 79435 | 17: 53532750 | 79447 | 17: 53919113 | 79454 | 17: 54025669 | 79455 | 17: 54029457 |
| 79456 | 17: 54030902 | 79457 | 17: 54033380 | 79458 | 17: 54062683 | 79459 | 17: 54122851 | 79461 | 17: 54128175 |
| 79465 | 17: 54163192 | 79468 | 17: 54264980 | 79469 | 17: 54267012 | 79470 | 17: 54310765 | 79483 | 17: 54668554 |
| 79489 | 17: 54807887 | 79499 | 17: 54895464 | 79505 | 17: 55074569 | 79506 | 17: 55081286 | 79507 | 17: 55082228 |
| 79508 | 17: 55105646 | 79509 | 17: 55105686 | 79512 | 17: 55385614 | 79517 | 17: 55484565 | 79521 | 17: 55592718 |
| 79523 | 17: 55665541 | 79524 | 17: 55716549 | 79526 | 17: 55752066 | 79532 | 17: 55876873 | 79537 | 17: 56011560 |
| 79548 | 17: 56279452 | 79549 | 17: 56281095 | 79560 | 17: 56566250 | 79564 | 17: 56651078 | 79565 | 17: 56710101 |
| 79571 | 17: 56976866 | 79576 | 17: 57116303 | 79583 | 17: 57285859 | 79591 | 17: 57472146 | 79598 | 17: 57643506 |
| 79601 | 17: 57716356 | 79602 | 17: 57730355 | 79603 | 17: 57731200 | 79610 | 17: 57797646 | 79612 | 17: 57831569 |
| 79623 | 17: 58093779 | 79625 | 17: 58112817 | 79626 | 17: 58130419 | 79627 | 17: 58141075 | 79634 | 17: 58304066 |
| 79665 | 17: 58556801 | 79670 | 17: 58775371 | 79679 | 17: 59008052 | 79688 | 17: 59157020 | 79690 | 17: 59215735 |
| 79700 | 17: 59384904 | 79702 | 17: 59397574 | 79710 | 17: 59604994 | 79714 | 17: 59716548 | 79719 | 17: 59839482 |
| 79730 | 17: 60067619 | 79731 | 17: 60090877 | 79732 | 17: 60090928 | 79733 | 17: 60097662 | 79743 | 17: 60248908 |
| 79751 | 17: 60373041 | 79752 | 17: 60374859 | 79753 | 17: 60395890 | 79754 | 17: 60395994 | 79760 | 17: 60500390 |
| 79771 | 17: 60710033 | 79772 | 17: 60710035 | 79773 | 17: 60710040 | 79774 | 17: 60710101 | 79779 | 17: 60795229 |
| 79780 | 17: 60795274 | 79781 | 17: 60795346 | 79784 | 17: 60843399 | 79807 | 17: 61141160 | 79812 | 17: 61202979 |
| 79814 | 17: 61208274 | 79815 | 17: 61208429 | 79816 | 17: 61237780 | 79819 | 17: 61316287 | 79822 | 17: 61370873 |
| 79828 | 17: 61499579 | 79829 | 17: 61499616 | 79842 | 17: 61892683 | 79846 | 17: 61971343 | 79850 | 17: 62029219 |
| 79858 | 17: 62310603 | 79866 | 17: 62556427 | 79874 | 17: 62749389 | 79882 | 17: 62937635 | 79884 | 17: 62967008 |
| 79886 | 17: 63060456 | 79890 | 17: 63193363 | 79904 | 17: 63343949 | 79909 | 17: 63512971 | 79912 | 17: 63562509 |
| 79916 | 17: 63612425 | 79917 | 17: 63613044 | 79918 | 17: 63614883 | 79919 | 17: 63615321 | 79920 | 17: 63615338 |
| 79921 | 17: 63619368 | 79922 | 17: 63619438 | 79923 | 17: 63619955 | 79926 | 17: 63623292 | 79927 | 17: 63624426 |
| 79933 | 17: 63696661 | 79937 | 17: 63745078 | 79938 | 17: 63765593 | 79946 | 17: 63909068 | 79947 | 17: 63911823 |
| 79948 | 17: 63914506 | 79949 | 17: 63924591 | 79953 | 17: 64073618 | 79954 | 17: 64077337 | 79955 | 17: 64079380 |
| 79957 | 17: 64164363 | 79959 | 17: 64233930 | 79963 | 17: 64320209 | 79964 | 17: 64364133 | 79965 | 17: 64376618 |
| 79966 | 17: 64381714 | 79967 | 17: 64390370 | 79968 | 17: 64391421 | 79969 | 17: 64392375 | 79971 | 17: 64400070 |
| 79975 | 17: 64543414 | 79983 | 17: 64868669 | 79986 | 17: 64936914 | 80004 | 17: 65250897 | 80005 | 17: 65252978 |
| 80007 | 17: 65269001 | 80009 | 17: 65311178 | 80015 | 17: 65495113 | 80016 | 17: 65515359 | 80028 | 17: 65711335 |
| 80046 | 17: 66084982 | 80048 | 17: 66098132 | 80091 | 17: 66926217 | 80093 | 17: 66962321 | 80094 | 17: 66963375 |
| 80095 | 17: 66964444 | 80096 | 17: 66980228 | 80097 | 17: 66981196 | 80098 | 17: 66983892 | 80099 | 17: 66987390 |
| 80100 | 17: 66989033 | 80101 | 17: 66991682 | 80102 | 17: 66993583 | 80117 | 17: 67452220 | 80120 | 17: 67493854 |
| 80123 | 17: 67576683 | 80133 | 17: 67991825 | 80143 | 17: 68190606 | 80145 | 17: 68235847 | 80146 | 17: 68237141 |
| 80152 | 17: 68336966 | 80154 | 17: 68390649 | 80156 | 17: 68404948 | 80165 | 17: 68610408 | 80169 | 17: 68711761 |
| 80177 | 17: 68924124 | 80186 | 17: 69025532 | 80187 | 17: 69025781 | 80191 | 17: 69104813 | 80200 | 17: 69235524 |
| 80201 | 17: 69244183 | 80208 | 17: 69526999 | 80209 | 17: 69557728 | 80210 | 17: 69558386 | 80221 | 17: 69723039 |
| 80226 | 17: 69823676 | 80236 | 17: 70089184 | 80242 | 17: 70183712 | 80266 | 17: 70611985 | 80267 | 17: 70615923 |
| 80268 | 17: 70617839 | 80269 | 17: 70618022 | 80270 | 17: 70618023 | 80271 | 17: 70619770 | 80272 | 17: 70621442 |
| 80294 | 17: 71216081 | 80295 | 17: 71224018 | 80297 | 17: 71275992 | 80298 | 17: 71276089 | 80299 | 17: 71276886 |
| 80300 | 17: 71276966 | 80301 | 17: 71283804 | 80306 | 17: 71441658 | 80309 | 17: 71512874 | 80310 | 17: 71518391 |
| 80311 | 17: 71528096 | 80312 | 17: 71528119 | 80313 | 17: 71528120 | 80314 | 17: 71528861 | 80315 | 17: 71528882 |
| 80316 | 17: 71528942 | 80324 | 17: 71695918 | 80325 | 17: 71702829 | 80328 | 17: 71812939 | 80339 | 17: 71951302 |
| 80349 | 17: 72204450 | 80356 | 17: 72342109 | 80358 | 17: 72363855 | 80360 | 17: 72369402 | 80362 | 17: 72398155 |
| 80364 | 17: 72408383 | 80365 | 17: 72445427 | 80366 | 17: 72447709 | 80367 | 17: 72457694 | 80369 | 17: 72468245 |
| 80370 | 17: 72498960 | 80374 | 17: 72555058 | 80384 | 17: 72883784 | 80386 | 17: 72916533 | 80391 | 17: 73036168 |
| 80411 | 18: 868580 | 80413 | 18: 911112 | 80414 | 18: 911196 | 80415 | 18: 932024 | 80416 | 18: 932036 |
| 80417 | 18: 932132 | 80431 | 18: 1178922 | 80435 | 18: 1215919 | 80436 | 18: 1269485 | 80437 | 18: 1269511 |
| 80438 | 18: 1269570 | 80452 | 18: 1553979 | 80453 | 18: 1565391 | 80460 | 18: 1658820 | 80467 | 18: 1733905 |
| 80468 | 18: 1734779 | 80469 | 18: 1737507 | 80470 | 18: 1739009 | 80471 | 18: 1741139 | 80472 | 18: 1742546 |
| 80474 | 18: 1743718 | 80475 | 18: 1744700 | 80476 | 18: 1745880 | 80477 | 18: 1746570 | 80478 | 18: 1747824 |
| 80479 | 18: 1749671 | 80480 | 18: 1751021 | 80481 | 18: 1752037 | 80482 | 18: 1753160 | 80483 | 18: 1754297 |
| 80484 | 18: 1754989 | 80485 | 18: 1755466 | 80486 | 18: 1758653 | 80487 | 18: 1759177 | 80488 | 18: 1759407 |
| 80489 | 18: 1759775 | 80490 | 18: 1760828 | 80491 | 18: 1761351 | 80492 | 18: 1761856 | 80493 | 18: 1764061 |
| 80494 | 18: 1765063 | 80495 | 18: 1765811 | 80496 | 18: 1766413 | 80497 | 18: 1766979 | 80498 | 18: 1780698 |
| 80499 | 18: 1799373 | 80503 | 18: 1821333 | 80515 | 18: 2132264 | 80519 | 18: 2189010 | 80525 | 18: 2310539 |
| 80529 | 18: 2407203 | 80532 | 18: 2547395 | 80535 | 18: 2627721 | 80548 | 18: 2801078 | 80549 | 18: 2824948 |
| 80553 | 18: 2893572 | 80559 | 18: 2948533 | 80590 | 18: 3732736 | 80593 | 18: 3803407 | 80599 | 18: 3918400 |
| 80604 | 18: 4117718 | 80610 | 18: 4175893 | 80622 | 18: 4456924 | 80623 | 18: 4458325 | 80652 | 18: 5105107 |
| 80657 | 18: 5261361 | 80658 | 18: 5303405 | 80667 | 18: 5381034 | 80668 | 18: 5392289 | 80686 | 18: 5796418 |
| 80695 | 18: 6075338 | 80699 | 18: 6140925 | 80700 | 18: 6150654 | 80702 | 18: 6155426 | 80708 | 18: 6179950 |
| 80710 | 18: 6195750 | 80717 | 18: 6307757 | 80732 | 18: 6411938 | 80733 | 18: 6414217 | 80734 | 18: 6434042 |
| 80736 | 18: 6473072 | 80742 | 18: 6606486 | 80756 | 18: 6904961 | 80759 | 18: 6952523 | 80775 | 18: 7393588 |
| 80782 | 18: 7571670 | 80783 | 18: 7571744 | 80784 | 18: 7571766 | 80790 | 18: 7722433 | 80791 | 18: 7725494 |
| 80792 | 18: 7759211 | 80794 | 18: 7785017 | 80795 | 18: 7806089 | 80798 | 18: 7857634 | 80809 | 18: 8151586 |
| 80814 | 18: 8174600 | 80816 | 18: 8217290 | 80817 | 18: 8219384 | 80818 | 18: 8219385 | 80819 | 18: 8219497 |
| 80836 | 18: 8556730 | 80847 | 18: 8895607 | 80850 | 18: 8930714 | 80868 | 18: 9094422 | 80869 | 18: 9121273 |
| 80871 | 18: 9155492 | 80873 | 18: 9160707 | 80877 | 18: 9224812 | 80879 | 18: 9281528 | 80891 | 18: 9500208 |
| 80893 | 18: 9514100 | 80897 | 18: 9541606 | 80898 | 18: 9560416 | 80903 | 18: 9622228 | 80913 | 18: 9939597 |
| 80915 | 18: 9939685 | 80920 | 18: 10053603 | 80937 | 18: 10466956 | 80938 | 18: 10467016 | 80939 | 18: 10474781 |
| 80940 | 18: 10474891 | 80944 | 18: 10554759 | 80945 | 18: 10555268 | 80949 | 18: 10623982 | 80954 | 18: 10638844 |
| 80955 | 18: 10661516 | 80959 | 18: 10775715 | 80966 | 18: 10924943 | 80970 | 18: 11032803 | 80972 | 18: 11076163 |
| 80975 | 18: 11143812 | 80978 | 18: 11177661 | 80979 | 18: 11189421 | 80982 | 18: 11197228 | 80985 | 18: 11315060 |
| 80986 | 18: 11322791 | 80987 | 18: 11333644 | 80988 | 18: 11340350 | 80989 | 18: 11349111 | 80990 | 18: 11352571 |
| 80991 | 18: 11362283 | 80992 | 18: 11367578 | 80993 | 18: 11398671 | 80995 | 18: 11499305 | 80996 | 18: 11499360 |
| 80997 | 18: 11589376 | 80999 | 18: 11631258 | 81001 | 18: 11639177 | 81002 | 18: 11640852 | 81004 | 18: 11652518 |
| 81007 | 18: 11719208 | 81026 | 18: 12374145 | 81030 | 18: 12487243 | 81036 | 18: 12677943 | 81058 | 18: 13316111 |
| 81059 | 18: 13322081 | 81060 | 18: 13326301 | 81068 | 18: 13483552 | 81090 | 18: 14035593 | 81092 | 18: 14117250 |
| 81097 | 18: 14266021 | 81104 | 18: 14579399 | 81110 | 18: 14880493 | 81111 | 18: 14918649 | 81121 | 18: 15157858 |
| 81129 | 18: 15352349 | 81130 | 18: 15353723 | 81136 | 18: 15529204 | 81137 | 18: 15532676 | 81138 | 18: 15535125 |
| 81139 | 18: 15537825 | 81142 | 18: 15574419 | 81145 | 18: 15607698 | 81147 | 18: 15643821 | 81149 | 18: 15771762 |
| 81163 | 18: 16246520 | 81164 | 18: 16246606 | 81165 | 18: 16246649 | 81167 | 18: 16294087 | 81170 | 18: 16339724 |
| 81179 | 18: 16491063 | 81186 | 18: 16701247 | 81188 | 18: 16722963 | 81191 | 18: 16855403 | 81194 | 18: 16929518 |
| 81198 | 18: 16960682 | 81205 | 18: 17095383 | 81206 | 18: 17095391 | 81207 | 18: 17095421 | 81208 | 18: 17128188 |
| 81214 | 18: 17231296 | 81219 | 18: 17332696 | 81224 | 18: 17474883 | 81225 | 18: 17474962 | 81246 | 18: 17964994 |
| 81248 | 18: 18015282 | 81259 | 18: 18234827 | 81274 | 18: 18340451 | 81277 | 18: 18455809 | 81281 | 18: 18555382 |
| 81286 | 18: 18634395 | 81288 | 18: 18652008 | 81292 | 18: 18693621 | 81296 | 18: 18741396 | 81297 | 18: 18766595 |
| 81304 | 18: 18991234 | 81308 | 18: 19028089 | 81312 | 18: 19104084 | 81318 | 18: 19279732 | 81319 | 18: 19306933 |
| 81321 | 18: 19321180 | 81328 | 18: 19549103 | 81337 | 18: 19828522 | 81341 | 18: 19884211 | 81346 | 18: 19992495 |
| 81379 | 18: 20783780 | 81396 | 18: 21231861 | 81397 | 18: 21231877 | 81404 | 18: 21429864 | 81405 | 18: 21429866 |
| 81419 | 18: 21805702 | 81422 | 18: 21842921 | 81427 | 18: 21915772 | 81428 | 18: 21944535 | 81429 | 18: 21944650 |
| 81439 | 18: 22075634 | 81459 | 18: 22432351 | 81467 | 18: 22574217 | 81468 | 18: 22581424 | 81470 | 18: 22616518 |
| 81489 | 18: 23048862 | 81515 | 18: 23639968 | 81523 | 18: 23885882 | 81526 | 18: 23978279 | 81530 | 18: 24024754 |
| 81531 | 18: 24028549 | 81532 | 18: 24039248 | 81538 | 18: 24159516 | 81541 | 18: 24233256 | 81547 | 18: 24449226 |
| 81548 | 18: 24467752 | 81551 | 18: 24569333 | 81555 | 18: 24603788 | 81556 | 18: 24605526 | 81557 | 18: 24606504 |
| 81558 | 18: 24609637 | 81559 | 18: 24622318 | 81560 | 18: 24626076 | 81561 | 18: 24627052 | 81562 | 18: 24640038 |
| 81563 | 18: 24646900 | 81564 | 18: 24648311 | 81565 | 18: 24650296 | 81566 | 18: 24653596 | 81567 | 18: 24655887 |
| 81568 | 18: 24658898 | 81569 | 18: 24680546 | 81572 | 18: 24703105 | 81573 | 18: 24711358 | 81581 | 18: 24916840 |
| 81586 | 18: 25087427 | 81594 | 18: 25243259 | 81595 | 18: 25263618 | 81597 | 18: 25320467 | 81603 | 18: 25475882 |
| 81604 | 18: 25507140 | 81608 | 18: 25560734 | 81613 | 18: 25671777 | 81614 | 18: 25693559 | 81617 | 18: 25769838 |
| 81618 | 18: 25772348 | 81619 | 18: 25777563 | 81622 | 18: 25833720 | 81626 | 18: 25944954 | 81638 | 18: 26118698 |
| 81643 | 18: 26252156 | 81648 | 18: 26367037 | 81652 | 18: 26448739 | 81674 | 18: 26911688 | 81683 | 18: 27123603 |
| 81711 | 18: 27938774 | 81712 | 18: 27938830 | 81733 | 18: 28523029 | 81752 | 18: 29298380 | 81753 | 18: 29324758 |
| 81755 | 18: 29354497 | 81756 | 18: 29354560 | 81757 | 18: 29354692 | 81758 | 18: 29356162 | 81759 | 18: 29359726 |
| 81760 | 18: 29359801 | 81765 | 18: 29507594 | 81766 | 18: 29507596 | 81767 | 18: 29507695 | 81768 | 18: 29517248 |
| 81800 | 18: 30444281 | 81812 | 18: 30824413 | 81820 | 18: 31114666 | 81821 | 18: 31161073 | 81829 | 18: 31403926 |
| 81860 | 18: 32193066 | 81865 | 18: 32277972 | 81866 | 18: 32277986 | 81889 | 18: 32769354 | 81892 | 18: 32797023 |
| 81893 | 18: 32809799 | 81909 | 18: 33174459 | 81913 | 18: 33266043 | 81915 | 18: 33332355 | 81928 | 18: 33524126 |
| 81938 | 18: 33726109 | 81942 | 18: 33803131 | 81943 | 18: 33819518 | 81948 | 18: 33891121 | 81950 | 18: 33985539 |
| 81958 | 18: 34273373 | 81960 | 18: 34305936 | 81962 | 18: 34318674 | 81963 | 18: 34340817 | 81967 | 18: 34415049 |
| 81968 | 18: 34460275 | 81969 | 18: 34460342 | 81971 | 18: 34477636 | 81973 | 18: 34507746 | 81976 | 18: 34573297 |
| 81979 | 18: 34674118 | 81981 | 18: 34742637 | 81985 | 18: 34868221 | 81993 | 18: 35213610 | 81994 | 18: 35247365 |
| 81996 | 18: 35258480 | 82001 | 18: 35371180 | 82003 | 18: 35406541 | 82008 | 18: 35505165 | 82009 | 18: 35515593 |
| 82010 | 18: 35575292 | 82012 | 18: 35602009 | 82016 | 18: 35713513 | 82021 | 18: 35869059 | 82027 | 18: 35949415 |
| 82029 | 18: 35961563 | 82035 | 18: 36058481 | 82036 | 18: 36063927 | 82038 | 18: 36087372 | 82039 | 18: 36087440 |
| 82040 | 18: 36093659 | 82042 | 18: 36121381 | 82046 | 18: 36318653 | 82054 | 18: 36585013 | 82059 | 18: 36712932 |
| 82066 | 18: 36883800 | 82067 | 18: 36890174 | 82069 | 18: 36892550 | 82093 | 18: 37455502 | 82098 | 18: 37533228 |
| 82102 | 18: 37619375 | 82122 | 18: 38001454 | 82131 | 18: 38196771 | 82133 | 18: 38218416 | 82135 | 18: 38277631 |
| 82138 | 18: 38301449 | 82141 | 18: 38331320 | 82150 | 18: 38614192 | 82174 | 18: 39233646 | 82175 | 18: 39286312 |
| 82180 | 18: 39414004 | 82182 | 18: 39447432 | 82189 | 18: 39589574 | 82192 | 18: 39612914 | 82215 | 18: 40137391 |
| 82216 | 18: 40137453 | 82217 | 18: 40138746 | 82218 | 18: 40138750 | 82219 | 18: 40138985 | 82220 | 18: 40139048 |
| 82222 | 18: 40176507 | 82223 | 18: 40176673 | 82228 | 18: 40316884 | 82230 | 18: 40330132 | 82237 | 18: 40533358 |
| 82238 | 18: 40534569 | 82241 | 18: 40597479 | 82249 | 18: 40763764 | 82250 | 18: 40763815 | 82254 | 18: 40819026 |
| 82255 | 18: 40819036 | 82261 | 18: 40956148 | 82269 | 18: 41180952 | 82274 | 18: 41372533 | 82275 | 18: 41391080 |
| 82277 | 18: 41412597 | 82304 | 18: 42002651 | 82308 | 18: 42064307 | 82314 | 18: 42154655 | 82325 | 18: 42312455 |
| 82342 | 18: 42673603 | 82352 | 18: 42839761 | 82355 | 18: 42870031 | 82356 | 18: 42870306 | 82357 | 18: 42870636 |
| 82373 | 18: 43209321 | 82378 | 18: 43300251 | 82397 | 18: 43597221 | 82399 | 18: 43723768 | 82400 | 18: 43729208 |
| 82401 | 18: 43744525 | 82403 | 18: 43765343 | 82415 | 18: 44100679 | 82418 | 18: 44219776 | 82433 | 18: 44678130 |
| 82437 | 18: 44746464 | 82440 | 18: 44842043 | 82444 | 18: 44999448 | 82448 | 18: 45230454 | 82451 | 18: 45235853 |
| 82453 | 18: 45282086 | 82454 | 18: 45368501 | 82457 | 18: 45512461 | 82459 | 18: 45555708 | 82460 | 18: 45555738 |
| 82461 | 18: 45555791 | 82463 | 18: 45568910 | 82474 | 18: 45823041 | 82477 | 18: 45919734 | 82481 | 18: 46058902 |
| 82482 | 18: 46058908 | 82483 | 18: 46059262 | 82484 | 18: 46059448 | 82485 | 18: 46059625 | 82486 | 18: 46074456 |
| 82488 | 18: 46098110 | 82491 | 18: 46151119 | 82493 | 18: 46204752 | 82500 | 18: 46339447 | 82506 | 18: 46411478 |
| 82507 | 18: 46412270 | 82510 | 18: 46452569 | 82511 | 18: 46454259 | 82514 | 18: 46462113 | 82515 | 18: 46472984 |
| 82516 | 18: 46474223 | 82517 | 18: 46474231 | 82518 | 18: 46474239 | 82519 | 18: 46474366 | 82520 | 18: 46480475 |
| 82521 | 18: 46481003 | 82522 | 18: 46481046 | 82523 | 18: 46481053 | 82524 | 18: 46481480 | 82525 | 18: 46481499 |
| 82526 | 18: 46489662 | 82527 | 18: 46490303 | 82528 | 18: 46490881 | 82529 | 18: 46498795 | 82530 | 18: 46499716 |
| 82531 | 18: 46500155 | 82532 | 18: 46511353 | 82533 | 18: 46512103 | 82534 | 18: 46512104 | 82535 | 18: 46523855 |
| 82536 | 18: 46528787 | 82540 | 18: 46627346 | 82542 | 18: 46696595 | 82543 | 18: 46697798 | 82544 | 18: 46727043 |
| 82546 | 18: 46727075 | 82547 | 18: 46731490 | 82548 | 18: 46731757 | 82549 | 18: 46731785 | 82550 | 18: 46731861 |
| 82551 | 18: 46742887 | 82552 | 18: 46742932 | 82553 | 18: 46742940 | 82554 | 18: 46743003 | 82555 | 18: 46745292 |
| 82556 | 18: 46745318 | 82557 | 18: 46745397 | 82558 | 18: 46745402 | 82559 | 18: 46747438 | 82560 | 18: 46747523 |
| 82561 | 18: 46751198 | 82562 | 18: 46751982 | 82563 | 18: 46751987 | 82564 | 18: 46752090 | 82565 | 18: 46754516 |
| 82567 | 18: 46762245 | 82568 | 18: 46762248 | 82569 | 18: 46763772 | 82570 | 18: 46764309 | 82571 | 18: 46764530 |
| 82572 | 18: 46764820 | 82573 | 18: 46764937 | 82574 | 18: 46765974 | 82575 | 18: 46766007 | 82576 | 18: 46766010 |
| 82577 | 18: 46771417 | 82578 | 18: 46772451 | 82581 | 18: 46793532 | 82585 | 18: 46926291 | 82586 | 18: 46926325 |
| 82587 | 18: 46935389 | 82588 | 18: 46936632 | 82589 | 18: 46936776 | 82592 | 18: 46961882 | 82593 | 18: 46987640 |
| 82606 | 18: 47331147 | 82607 | 18: 47350791 | 82609 | 18: 47380300 | 82614 | 18: 47561882 | 82615 | 18: 47569792 |
| 82616 | 18: 47584273 | 82623 | 18: 47724055 | 82630 | 18: 47880052 | 82632 | 18: 47904454 | 82633 | 18: 47905402 |
| 82635 | 18: 47914575 | 82638 | 18: 47982264 | 82639 | 18: 47986634 | 82645 | 18: 48080577 | 82646 | 18: 48082036 |
| 82647 | 18: 48092307 | 82648 | 18: 48093267 | 82649 | 18: 48094554 | 82650 | 18: 48095808 | 82651 | 18: 48097168 |
| 82652 | 18: 48098873 | 82653 | 18: 48099751 | 82654 | 18: 48100359 | 82655 | 18: 48102403 | 82656 | 18: 48103383 |
| 82657 | 18: 48105090 | 82658 | 18: 48105806 | 82660 | 18: 48115016 | 82661 | 18: 48119818 | 82662 | 18: 48164016 |
| 82669 | 18: 48328458 | 82674 | 18: 48486626 | 82677 | 18: 48540681 | 82678 | 18: 48540711 | 82681 | 18: 48550284 |
| 82682 | 18: 48552506 | 82684 | 18: 48648276 | 82685 | 18: 48661989 | 82692 | 18: 48824317 | 82700 | 18: 49125312 |
| 82709 | 18: 49431844 | 82710 | 18: 49440743 | 82718 | 18: 49685722 | 82720 | 18: 49690710 | 82722 | 18: 49774209 |
| 82727 | 18: 49905675 | 82734 | 18: 49974216 | 82744 | 18: 50307933 | 82768 | 18: 50909576 | 82776 | 18: 51192874 |
| 82780 | 18: 51273762 | 82784 | 18: 51393314 | 82799 | 18: 51647623 | 82801 | 18: 51684659 | 82803 | 18: 51687197 |
| 82804 | 18: 51691345 | 82805 | 18: 51691402 | 82806 | 18: 51691426 | 82807 | 18: 51691907 | 82808 | 18: 51692088 |
| 82810 | 18: 51703006 | 82811 | 18: 51703434 | 82813 | 18: 51710422 | 82820 | 18: 51868158 | 82821 | 18: 51868247 |
| 82822 | 18: 51877833 | 82823 | 18: 51888735 | 82826 | 18: 51959716 | 82833 | 18: 52170747 | 82843 | 18: 52408540 |
| 82844 | 18: 52434788 | 82846 | 18: 52463921 | 82848 | 18: 52526615 | 82849 | 18: 52569137 | 82850 | 18: 52586631 |
| 82856 | 18: 52750690 | 82863 | 18: 53056273 | 82864 | 18: 53066684 | 82876 | 18: 53447947 | 82877 | 18: 53462716 |
| 82878 | 18: 53515653 | 82880 | 18: 53545259 | 82883 | 18: 53565634 | 82885 | 18: 53596284 | 82889 | 18: 53631016 |
| 82890 | 18: 53635483 | 82891 | 18: 53666625 | 82892 | 18: 53680050 | 82902 | 18: 53873089 | 82903 | 18: 53882150 |
| 82907 | 18: 53924797 | 82908 | 18: 53926298 | 82909 | 18: 53938427 | 82910 | 18: 53965820 | 82911 | 18: 53970987 |
| 82912 | 18: 53987026 | 82913 | 18: 53988178 | 82917 | 18: 54051234 | 82922 | 18: 54129853 | 82924 | 18: 54256059 |
| 82929 | 18: 54326329 | 82930 | 18: 54348038 | 82931 | 18: 54348158 | 82932 | 18: 54356059 | 82943 | 18: 54594314 |
| 82945 | 18: 54606774 | 82946 | 18: 54607245 | 82947 | 18: 54615284 | 82948 | 18: 54615550 | 82949 | 18: 54618878 |
| 82951 | 18: 54632037 | 82952 | 18: 54674333 | 82956 | 18: 54725999 | 82957 | 18: 54726122 | 82958 | 18: 54732855 |
| 82965 | 18: 54920625 | 82978 | 18: 55105119 | 82980 | 18: 55125421 | 82981 | 18: 55130089 | 82991 | 18: 55397342 |
| 82993 | 18: 55448102 | 82994 | 18: 55479044 | 83003 | 18: 55722088 | 83006 | 18: 55855289 | 83010 | 18: 55908909 |
| 83022 | 18: 56235646 | 83025 | 18: 56256914 | 83027 | 18: 56286701 | 83028 | 18: 56286760 | 83030 | 18: 56311848 |
| 83037 | 18: 56458961 | 83039 | 18: 56480496 | 83040 | 18: 56481635 | 83041 | 18: 56500189 | 83043 | 18: 56516815 |
| 83046 | 18: 56550331 | 83051 | 18: 56657542 | 83053 | 18: 56669005 | 83054 | 18: 56669128 | 83055 | 18: 56679475 |
| 83114 | 18: 57068537 | 83115 | 18: 57072585 | 83119 | 18: 57151314 | 83120 | 18: 57195331 | 83126 | 18: 57435685 |
| 83134 | 18: 57498462 | 83140 | 18: 57652070 | 83141 | 18: 57658393 | 83142 | 18: 57662151 | 83143 | 18: 57673429 |
| 83149 | 18: 57750585 | 83150 | 18: 57764229 | 83151 | 18: 57774781 | 83159 | 18: 57875057 | 83160 | 18: 57880006 |
| 83162 | 18: 57922196 | 83163 | 18: 57922208 | 83168 | 18: 57950000 | 83184 | 18: 58213698 | 83185 | 18: 58215105 |
| 83186 | 18: 58221738 | 83187 | 18: 58237470 | 83189 | 18: 58270823 | 83190 | 18: 58280185 | 83191 | 18: 58285064 |
| 83192 | 18: 58286528 | 83193 | 18: 58288374 | 83194 | 18: 58289386 | 83195 | 18: 58290105 | 83196 | 18: 58292173 |
| 83197 | 18: 58292702 | 83198 | 18: 58293461 | 83199 | 18: 58294515 | 83200 | 18: 58296803 | 83201 | 18: 58297833 |
| 83202 | 18: 58298676 | 83203 | 18: 58302578 | 83204 | 18: 58303580 | 83205 | 18: 58304589 | 83206 | 18: 58306121 |
| 83207 | 18: 58308817 | 83208 | 18: 58309408 | 83209 | 18: 58311748 | 83210 | 18: 58314758 | 83211 | 18: 58319283 |
| 83213 | 18: 58328404 | 83214 | 18: 58335688 | 83215 | 18: 58336548 | 83217 | 18: 58339398 | 83218 | 18: 58340297 |
| 83219 | 18: 58341452 | 83220 | 18: 58343184 | 83221 | 18: 58346217 | 83222 | 18: 58359203 | 83223 | 18: 58360688 |
| 83224 | 18: 58362462 | 83225 | 18: 58369410 | 83226 | 18: 58372976 | 83228 | 18: 58501449 | 83259 | 18: 59941034 |
| 83264 | 18: 60057741 | 83276 | 18: 60557876 | 83284 | 18: 60892284 | 83285 | 18: 60892325 | 83286 | 18: 60892345 |
| 83287 | 18: 60908498 | 83296 | 18: 61230100 | 83297 | 18: 61248798 | 83306 | 18: 61528542 | 83314 | 18: 61669548 |
| 83316 | 18: 61688550 | 83327 | 18: 61924215 | 83328 | 18: 61925438 | 83329 | 18: 61925595 | 83331 | 18: 61938092 |
| 83332 | 18: 61941006 | 83333 | 18: 61948334 | 83334 | 18: 61949940 | 83336 | 18: 61962954 | 83337 | 18: 61968568 |
| 83338 | 18: 61969417 | 83339 | 18: 61975282 | 83340 | 18: 61976235 | 83341 | 18: 61977792 | 83343 | 18: 61979272 |
| 83344 | 18: 61986664 | 83345 | 18: 61987641 | 83347 | 18: 62017097 | 83348 | 18: 62031220 | 83350 | 18: 62050366 |
| 83351 | 18: 62055500 | 83352 | 18: 62069703 | 83355 | 18: 62098858 | 83356 | 18: 62119696 | 83365 | 18: 62301361 |
| 83369 | 18: 62396313 | 83370 | 18: 62411556 | 83371 | 18: 62412333 | 83372 | 18: 62491921 | 83391 | 18: 63189938 |
| 83392 | 18: 63193118 | 83393 | 18: 63203763 | 83396 | 18: 63217724 | 83397 | 18: 63217760 | 83398 | 18: 63217761 |
| 83401 | 18: 63282873 | 83406 | 18: 63442841 | 83407 | 18: 63443616 | 83410 | 18: 63449366 | 83413 | 18: 63478461 |
| 83420 | 18: 63566906 | 83435 | 18: 63851165 | 83438 | 18: 63870217 | 83439 | 18: 63870560 | 83440 | 18: 63870634 |
| 83441 | 18: 63878287 | 83446 | 18: 64020285 | 83448 | 18: 64103695 | 83449 | 18: 64107247 | 83450 | 18: 64118087 |
| 83459 | 18: 64261304 | 83464 | 18: 64351790 | 83473 | 18: 64453963 | 83475 | 18: 64457710 | 83480 | 18: 64586875 |
| 83481 | 18: 64586957 | 83483 | 18: 64587029 | 83484 | 18: 64588207 | 83485 | 18: 64588277 | 83486 | 18: 64594228 |
| 83489 | 18: 64607295 | 83493 | 18: 64684413 | 83500 | 18: 64871544 | 83506 | 18: 64979546 | 83507 | 18: 64989257 |
| 83508 | 18: 65000708 | 83512 | 18: 65024355 | 83513 | 18: 65052564 | 83514 | 18: 65058054 | 83515 | 18: 65058108 |
| 83516 | 18: 65058135 | 83522 | 18: 65143297 | 83538 | 18: 65213339 | 83546 | 18: 65269401 | 83570 | 18: 65467804 |
| 83573 | 18: 65499590 | 83576 | 18: 65531536 | 83582 | 18: 65680076 | 83584 | 18: 65778621 | 83611 | 19: 1044735 |
| 83641 | 19: 2028992 | 83643 | 19: 2087607 | 83647 | 19: 2189142 | 83648 | 19: 2189181 | 83669 | 19: 3019557 |
| 83676 | 19: 3224135 | 83684 | 19: 3507467 | 83688 | 19: 3693922 | 83689 | 19: 3693998 | 83690 | 19: 3694048 |
| 83691 | 19: 3717004 | 83712 | 19: 4248938 | 83719 | 19: 4532769 | 83732 | 19: 4885934 | 83737 | 19: 5057366 |
| 83738 | 19: 5077598 | 83742 | 19: 5131889 | 83743 | 19: 5144117 | 83748 | 19: 5302256 | 83758 | 19: 5544452 |
| 83759 | 19: 5550410 | 83770 | 19: 5841378 | 83773 | 19: 5911200 | 83777 | 19: 6008981 | 83779 | 19: 6069254 |
| 83780 | 19: 6080767 | 83802 | 19: 6741959 | 83803 | 19: 6742031 | 83804 | 19: 6744112 | 83808 | 19: 6821054 |
| 83810 | 19: 6854445 | 83823 | 19: 7084364 | 83825 | 19: 7139360 | 83830 | 19: 7250907 | 83835 | 19: 7311199 |
| 83857 | 19: 7610709 | 83860 | 19: 7655487 | 83867 | 19: 7877242 | 83870 | 19: 7955627 | 83874 | 19: 8012167 |
| 83882 | 19: 8102634 | 83884 | 19: 8134333 | 83888 | 19: 8242768 | 83890 | 19: 8251385 | 83891 | 19: 8253685 |
| 83892 | 19: 8253738 | 83893 | 19: 8256106 | 83894 | 19: 8257817 | 83896 | 19: 8303146 | 83897 | 19: 8307007 |
| 83899 | 19: 8318345 | 83909 | 19: 8375150 | 83914 | 19: 8447018 | 83917 | 19: 8481149 | 83919 | 19: 8538010 |
| 83928 | 19: 8786239 | 83937 | 19: 8921388 | 83938 | 19: 8940266 | 83939 | 19: 8940272 | 83946 | 19: 9148135 |
| 83948 | 19: 9161538 | 83951 | 19: 9186570 | 83952 | 19: 9186624 | 83953 | 19: 9186684 | 83954 | 19: 9186696 |
| 83955 | 19: 9186752 | 83960 | 19: 9321464 | 83963 | 19: 9325083 | 83964 | 19: 9329176 | 83971 | 19: 9494606 |
| 83973 | 19: 9527082 | 83974 | 19: 9549010 | 83976 | 19: 9584099 | 83978 | 19: 9638098 | 83996 | 19: 10144335 |
| 84000 | 19: 10161366 | 84001 | 19: 10185318 | 84002 | 19: 10193665 | 84004 | 19: 10203916 | 84005 | 19: 10212410 |
| 84006 | 19: 10222141 | 84012 | 19: 10311673 | 84015 | 19: 10357642 | 84018 | 19: 10410339 | 84027 | 19: 10608070 |
| 84028 | 19: 10663875 | 84033 | 19: 10816146 | 84048 | 19: 11195343 | 84049 | 19: 11206085 | 84050 | 19: 11223181 |
| 84051 | 19: 11223607 | 84074 | 19: 11794721 | 84081 | 19: 12080713 | 84098 | 19: 12520276 | 84106 | 19: 12624098 |
| 84115 | 19: 12851003 | 84118 | 19: 12942065 | 84119 | 19: 12942066 | 84138 | 19: 13385076 | 84153 | 19: 13539153 |
| 84154 | 19: 13540416 | 84156 | 19: 13546434 | 84157 | 19: 13553916 | 84158 | 19: 13554659 | 84160 | 19: 13567678 |
| 84161 | 19: 13567681 | 84162 | 19: 13569519 | 84163 | 19: 13589753 | 84167 | 19: 13627880 | 84168 | 19: 13629329 |
| 84182 | 19: 13922585 | 84184 | 19: 13992338 | 84185 | 19: 13992353 | 84193 | 19: 14098428 | 84197 | 19: 14234372 |
| 84199 | 19: 14285305 | 84202 | 19: 14359808 | 84206 | 19: 14455998 | 84207 | 19: 14458140 | 84208 | 19: 14460416 |
| 84209 | 19: 14461216 | 84212 | 19: 14524347 | 84215 | 19: 14594291 | 84231 | 19: 14987147 | 84233 | 19: 14997330 |
| 84236 | 19: 15044294 | 84238 | 19: 15079297 | 84265 | 19: 15653982 | 84275 | 19: 15950588 | 84282 | 19: 16153652 |
| 84285 | 19: 16179422 | 84286 | 19: 16179458 | 84290 | 19: 16243199 | 84291 | 19: 16245110 | 84294 | 19: 16316143 |
| 84296 | 19: 16414613 | 84297 | 19: 16414653 | 84298 | 19: 16414656 | 84299 | 19: 16414688 | 84300 | 19: 16414750 |
| 84301 | 19: 16414769 | 84305 | 19: 16538181 | 84306 | 19: 16538222 | 84307 | 19: 16538228 | 84308 | 19: 16538286 |
| 84309 | 19: 16538332 | 84314 | 19: 16680352 | 84315 | 19: 16698614 | 84316 | 19: 16701167 | 84317 | 19: 16704714 |
| 84318 | 19: 16710947 | 84319 | 19: 16712329 | 84320 | 19: 16715359 | 84329 | 19: 16900771 | 84344 | 19: 17139083 |
| 84345 | 19: 17193285 | 84347 | 19: 17219724 | 84354 | 19: 17346967 | 84358 | 19: 17413575 | 84360 | 19: 17439500 |
| 84361 | 19: 17463604 | 84362 | 19: 17463752 | 84363 | 19: 17463766 | 84364 | 19: 17463785 | 84366 | 19: 17476802 |
| 84367 | 19: 17485925 | 84381 | 19: 17839100 | 84382 | 19: 17840344 | 84383 | 19: 17847133 | 84388 | 19: 17935227 |
| 84390 | 19: 18002956 | 84391 | 19: 18012136 | 84405 | 19: 18364670 | 84427 | 19: 18812123 | 84429 | 19: 18935333 |
| 84434 | 19: 19027484 | 84438 | 19: 19066522 | 84440 | 19: 19164165 | 84441 | 19: 19167861 | 84443 | 19: 19248544 |
| 84448 | 19: 19342984 | 84455 | 19: 19497354 | 84456 | 19: 19503339 | 84469 | 19: 19832430 | 84470 | 19: 19832910 |
| 84473 | 19: 19891932 | 84478 | 19: 20010169 | 84479 | 19: 20010699 | 84480 | 19: 20021077 | 84481 | 19: 20024935 |
| 84482 | 19: 20039821 | 84483 | 19: 20039838 | 84484 | 19: 20049560 | 84486 | 19: 20064375 | 84487 | 19: 20065239 |
| 84488 | 19: 20065746 | 84489 | 19: 20067184 | 84490 | 19: 20067197 | 84496 | 19: 20169459 | 84497 | 19: 20252737 |
| 84498 | 19: 20269891 | 84499 | 19: 20272304 | 84500 | 19: 20272993 | 84501 | 19: 20272997 | 84502 | 19: 20287832 |
| 84503 | 19: 20347363 | 84504 | 19: 20347364 | 84505 | 19: 20347398 | 84506 | 19: 20347408 | 84507 | 19: 20347436 |
| 84508 | 19: 20347583 | 84511 | 19: 20397604 | 84513 | 19: 20404265 | 84514 | 19: 20406390 | 84515 | 19: 20407245 |
| 84516 | 19: 20407776 | 84517 | 19: 20407895 | 84518 | 19: 20408583 | 84519 | 19: 20409859 | 84520 | 19: 20410892 |
| 84521 | 19: 20411434 | 84522 | 19: 20412101 | 84523 | 19: 20413435 | 84524 | 19: 20414800 | 84525 | 19: 20415545 |
| 84526 | 19: 20415733 | 84527 | 19: 20416065 | 84528 | 19: 20416081 | 84529 | 19: 20416676 | 84530 | 19: 20417918 |
| 84531 | 19: 20418417 | 84532 | 19: 20418508 | 84533 | 19: 20418613 | 84535 | 19: 20421499 | 84536 | 19: 20423129 |
| 84537 | 19: 20427231 | 84538 | 19: 20443801 | 84539 | 19: 20448136 | 84540 | 19: 20449515 | 84541 | 19: 20450452 |
| 84542 | 19: 20451130 | 84543 | 19: 20454459 | 84544 | 19: 20459782 | 84545 | 19: 20464970 | 84546 | 19: 20466789 |
| 84547 | 19: 20468157 | 84548 | 19: 20469610 | 84557 | 19: 20885539 | 84561 | 19: 20948913 | 84570 | 19: 21194708 |
| 84571 | 19: 21195341 | 84580 | 19: 21541775 | 84582 | 19: 21606050 | 84583 | 19: 21606066 | 84584 | 19: 21606170 |
| 84586 | 19: 21607755 | 84587 | 19: 21607870 | 84588 | 19: 21657448 | 84596 | 19: 21761419 | 84601 | 19: 21810539 |
| 84602 | 19: 21810883 | 84608 | 19: 22007200 | 84613 | 19: 22125908 | 84617 | 19: 22209844 | 84618 | 19: 22216194 |
| 84619 | 19: 22216936 | 84620 | 19: 22218946 | 84621 | 19: 22223761 | 84622 | 19: 22225302 | 84624 | 19: 22228117 |
| 84625 | 19: 22228881 | 84626 | 19: 22238525 | 84629 | 19: 22288784 | 84632 | 19: 22387067 | 84637 | 19: 22435985 |
| 84640 | 19: 22580315 | 84642 | 19: 22630921 | 84645 | 19: 22686577 | 84649 | 19: 22777911 | 84653 | 19: 22919648 |
| 84654 | 19: 22933829 | 84655 | 19: 22937788 | 84659 | 19: 23047864 | 84663 | 19: 23089782 | 84665 | 19: 23103859 |
| 84666 | 19: 23125220 | 84677 | 19: 23322846 | 84678 | 19: 23328857 | 84691 | 19: 23748456 | 84692 | 19: 23762942 |
| 84696 | 19: 23795890 | 84699 | 19: 23844740 | 84709 | 19: 24085976 | 84710 | 19: 24086312 | 84711 | 19: 24086389 |
| 84712 | 19: 24100573 | 84713 | 19: 24119941 | 84714 | 19: 24120015 | 84715 | 19: 24120089 | 84716 | 19: 24120654 |
| 84717 | 19: 24120726 | 84719 | 19: 24158070 | 84720 | 19: 24171676 | 84721 | 19: 24190844 | 84722 | 19: 24190846 |
| 84723 | 19: 24190851 | 84724 | 19: 24190893 | 84725 | 19: 24201030 | 84726 | 19: 24201096 | 84727 | 19: 24203359 |
| 84728 | 19: 24203402 | 84729 | 19: 24203454 | 84730 | 19: 24204657 | 84731 | 19: 24206255 | 84732 | 19: 24206362 |
| 84733 | 19: 24210049 | 84734 | 19: 24211193 | 84740 | 19: 24352581 | 84749 | 19: 24484569 | 84752 | 19: 24575685 |
| 84757 | 19: 24628286 | 84758 | 19: 24643342 | 84760 | 19: 24654902 | 84763 | 19: 24710033 | 84766 | 19: 24729440 |
| 84767 | 19: 24730057 | 84768 | 19: 24732020 | 84769 | 19: 24736477 | 84770 | 19: 24737388 | 84771 | 19: 24739849 |
| 84772 | 19: 24743924 | 84773 | 19: 24747961 | 84774 | 19: 24753662 | 84776 | 19: 24761701 | 84777 | 19: 24791465 |
| 84778 | 19: 24832457 | 84779 | 19: 24834421 | 84780 | 19: 24843420 | 84782 | 19: 24848682 | 84783 | 19: 24859742 |
| 84784 | 19: 24862477 | 84787 | 19: 24938845 | 84788 | 19: 24958116 | 84789 | 19: 24958789 | 84793 | 19: 25015319 |
| 84795 | 19: 25042003 | 84797 | 19: 25049455 | 84799 | 19: 25055788 | 84802 | 19: 25112769 | 84808 | 19: 25148679 |
| 84809 | 19: 25149070 | 84810 | 19: 25167024 | 84815 | 19: 25292038 | 84843 | 19: 25636984 | 84847 | 19: 25714307 |
| 84866 | 19: 26042854 | 84867 | 19: 26047036 | 84868 | 19: 26047065 | 84869 | 19: 26047497 | 84870 | 19: 26047588 |
| 84871 | 19: 26048186 | 84872 | 19: 26048187 | 84873 | 19: 26048200 | 84874 | 19: 26048237 | 84875 | 19: 26048249 |
| 84876 | 19: 26050069 | 84877 | 19: 26050076 | 84878 | 19: 26050122 | 84894 | 19: 26326260 | 84895 | 19: 26328504 |
| 84896 | 19: 26330382 | 84897 | 19: 26362443 | 84903 | 19: 26386801 | 84909 | 19: 26431555 | 84911 | 19: 26433035 |
| 84913 | 19: 26435773 | 84914 | 19: 26437827 | 84915 | 19: 26438388 | 84919 | 19: 26443714 | 84920 | 19: 26452130 |
| 84921 | 19: 26455955 | 84923 | 19: 26459081 | 84924 | 19: 26461611 | 84929 | 19: 26467372 | 84933 | 19: 26494500 |
| 84934 | 19: 26498170 | 84937 | 19: 26508308 | 84938 | 19: 26509449 | 84940 | 19: 26512520 | 84943 | 19: 26571898 |
| 84967 | 19: 26833517 | 84968 | 19: 26834363 | 84971 | 19: 26944800 | 84972 | 19: 26944904 | 84991 | 19: 27851735 |
| 84994 | 19: 27919465 | 84995 | 19: 27929802 | 84997 | 19: 27974645 | 84999 | 19: 27986943 | 85003 | 19: 28063061 |
| 85022 | 19: 28341985 | 85027 | 19: 28441795 | 85033 | 19: 28599438 | 85039 | 19: 28789212 | 85055 | 19: 29142555 |
| 85056 | 19: 29146537 | 85057 | 19: 29165645 | 85060 | 19: 29185159 | 85062 | 19: 29191189 | 85070 | 19: 29360624 |
| 85071 | 19: 29360635 | 85077 | 19: 29516407 | 85082 | 19: 29601656 | 85088 | 19: 29667582 | 85089 | 19: 29671837 |
| 85093 | 19: 29784730 | 85094 | 19: 29784751 | 85095 | 19: 29784815 | 85106 | 19: 30101489 | 85109 | 19: 30199565 |
| 85125 | 19: 30493276 | 85143 | 19: 30892000 | 85145 | 19: 30952848 | 85148 | 19: 31087410 | 85150 | 19: 31146132 |
| 85153 | 19: 31161313 | 85154 | 19: 31165405 | 85155 | 19: 31165486 | 85160 | 19: 31346087 | 85161 | 19: 31369446 |
| 85193 | 19: 32163965 | 85194 | 19: 32172115 | 85201 | 19: 32267525 | 85223 | 19: 32751602 | 85224 | 19: 32768693 |
| 85225 | 19: 32800795 | 85226 | 19: 32804878 | 85233 | 19: 33007630 | 85234 | 19: 33019809 | 85238 | 19: 33125153 |
| 85243 | 19: 33187115 | 85246 | 19: 33215711 | 85249 | 19: 33262257 | 85255 | 19: 33294100 | 85267 | 19: 33578331 |
| 85268 | 19: 33594931 | 85269 | 19: 33596340 | 85271 | 19: 33623310 | 85279 | 19: 33954621 | 85281 | 19: 33998092 |
| 85282 | 19: 34005543 | 85283 | 19: 34005879 | 85286 | 19: 34041049 | 85303 | 19: 34565878 | 85306 | 19: 34576319 |
| 85313 | 19: 34769105 | 85314 | 19: 34779947 | 85315 | 19: 34782489 | 85316 | 19: 34786152 | 85317 | 19: 34795515 |
| 85318 | 19: 34801199 | 85319 | 19: 34814559 | 85320 | 19: 34815409 | 85321 | 19: 34821534 | 85322 | 19: 34822575 |
| 85323 | 19: 34823488 | 85324 | 19: 34824385 | 85325 | 19: 34825185 | 85326 | 19: 34826088 | 85327 | 19: 34826902 |
| 85328 | 19: 34827822 | 85329 | 19: 34832510 | 85330 | 19: 34833809 | 85331 | 19: 34835066 | 85332 | 19: 34839003 |
| 85333 | 19: 34839771 | 85334 | 19: 34840668 | 85335 | 19: 34842069 | 85336 | 19: 34843150 | 85337 | 19: 34850412 |
| 85338 | 19: 34851901 | 85339 | 19: 34860953 | 85340 | 19: 34867998 | 85341 | 19: 34873374 | 85342 | 19: 34876608 |
| 85343 | 19: 34877505 | 85344 | 19: 34884414 | 85346 | 19: 34893695 | 85348 | 19: 34911757 | 85349 | 19: 34917689 |
| 85350 | 19: 34924937 | 85351 | 19: 34940577 | 85352 | 19: 34941647 | 85353 | 19: 34942605 | 85360 | 19: 35015390 |
| 85385 | 19: 35630509 | 85391 | 19: 35778297 | 85404 | 19: 36050548 | 85405 | 19: 36052759 | 85408 | 19: 36139902 |
| 85410 | 19: 36161222 | 85412 | 19: 36183218 | 85413 | 19: 36186920 | 85414 | 19: 36204759 | 85415 | 19: 36205924 |
| 85417 | 19: 36230644 | 85418 | 19: 36232203 | 85419 | 19: 36233310 | 85420 | 19: 36234144 | 85421 | 19: 36235392 |
| 85422 | 19: 36236022 | 85423 | 19: 36236961 | 85424 | 19: 36238112 | 85425 | 19: 36239325 | 85426 | 19: 36242008 |
| 85430 | 19: 36285683 | 85432 | 19: 36304660 | 85436 | 19: 36349079 | 85443 | 19: 36515692 | 85444 | 19: 36516381 |
| 85445 | 19: 36540830 | 85446 | 19: 36572979 | 85447 | 19: 36574100 | 85448 | 19: 36574859 | 85449 | 19: 36574986 |
| 85450 | 19: 36576060 | 85451 | 19: 36577180 | 85452 | 19: 36577974 | 85453 | 19: 36585052 | 85454 | 19: 36585096 |
| 85455 | 19: 36585153 | 85456 | 19: 36586555 | 85457 | 19: 36588245 | 85458 | 19: 36598037 | 85460 | 19: 36659503 |
| 85478 | 19: 37057391 | 85480 | 19: 37085911 | 85503 | 19: 37768695 | 85512 | 19: 38027080 | 85513 | 19: 38112442 |
| 85514 | 19: 38115441 | 85515 | 19: 38135659 | 85519 | 19: 38330215 | 85523 | 19: 38378249 | 85535 | 19: 38818642 |
| 85537 | 19: 38825404 | 85543 | 19: 39018067 | 85546 | 19: 39173581 | 85548 | 19: 39195126 | 85554 | 19: 39319605 |
| 85555 | 19: 39319939 | 85556 | 19: 39324677 | 85557 | 19: 39373787 | 85558 | 19: 39403214 | 85560 | 19: 39423726 |
| 85564 | 19: 39558542 | 85568 | 19: 39592912 | 85572 | 19: 39664178 | 85574 | 19: 39702056 | 85575 | 19: 39761996 |
| 85585 | 19: 40136281 | 85589 | 19: 40231461 | 85591 | 19: 40243945 | 85592 | 19: 40244440 | 85593 | 19: 40244746 |
| 85594 | 19: 40250106 | 85603 | 19: 40340447 | 85608 | 19: 40504497 | 85611 | 19: 40571544 | 85617 | 19: 40687629 |
| 85634 | 19: 40969791 | 85636 | 19: 40998269 | 85637 | 19: 41009813 | 85639 | 19: 41075395 | 85641 | 19: 41125615 |
| 85642 | 19: 41125654 | 85643 | 19: 41126901 | 85644 | 19: 41134261 | 85645 | 19: 41134335 | 85646 | 19: 41167136 |
| 85666 | 19: 41672120 | 85671 | 19: 41759325 | 85681 | 19: 41844566 | 85683 | 19: 41866585 | 85685 | 19: 41908524 |
| 85687 | 19: 41944555 | 85689 | 19: 41954155 | 85694 | 19: 42058566 | 85695 | 19: 42063521 | 85698 | 19: 42131015 |
| 85701 | 19: 42144465 | 85704 | 19: 42227260 | 85710 | 19: 42333202 | 85712 | 19: 42345437 | 85719 | 19: 42426465 |
| 85728 | 19: 42644624 | 85731 | 19: 42719942 | 85732 | 19: 42726195 | 85736 | 19: 42848843 | 85738 | 19: 42887598 |
| 85741 | 19: 43035501 | 85742 | 19: 43043947 | 85743 | 19: 43049014 | 85750 | 19: 43333514 | 85759 | 19: 43422824 |
| 85768 | 19: 43443947 | 85775 | 19: 43599080 | 85777 | 19: 43713425 | 85783 | 19: 43816258 | 85784 | 19: 43830215 |
| 85787 | 19: 43926648 | 85790 | 19: 43991252 | 85793 | 19: 44072509 | 85795 | 19: 44116524 | 85797 | 19: 44122310 |
| 85799 | 19: 44173520 | 85800 | 19: 44177664 | 85801 | 19: 44178974 | 85806 | 19: 44309222 | 85808 | 19: 44359879 |
| 85809 | 19: 44385146 | 85813 | 19: 44477082 | 85818 | 19: 44577139 | 85821 | 19: 44615861 | 85822 | 19: 44651270 |
| 85823 | 19: 44673344 | 85824 | 19: 44673867 | 85826 | 19: 44715813 | 85838 | 19: 45013727 | 85841 | 19: 45073651 |
| 85861 | 19: 45630233 | 85892 | 19: 45816366 | 85893 | 19: 45817041 | 85896 | 19: 45844326 | 85898 | 19: 45920605 |
| 85899 | 19: 45934999 | 85900 | 19: 45949710 | 85909 | 19: 46106226 | 85918 | 19: 46396057 | 85928 | 19: 46762829 |
| 85933 | 19: 46867389 | 85944 | 19: 47138439 | 85945 | 19: 47138441 | 85946 | 19: 47138654 | 85947 | 19: 47138655 |
| 85948 | 19: 47138674 | 85949 | 19: 47138684 | 85950 | 19: 47139281 | 85958 | 19: 47384175 | 85959 | 19: 47402508 |
| 85960 | 19: 47405017 | 85974 | 19: 47676904 | 85975 | 19: 47735369 | 85979 | 19: 47763256 | 85984 | 19: 47930272 |
| 85986 | 19: 47955876 | 85987 | 19: 47966555 | 85990 | 19: 48025233 | 85993 | 19: 48071339 | 85994 | 19: 48075239 |
| 85996 | 19: 48084284 | 86000 | 19: 48118256 | 86001 | 19: 48142220 | 86005 | 19: 48258274 | 86006 | 19: 48266963 |
| 86012 | 19: 48389397 | 86016 | 19: 48418566 | 86022 | 19: 48563047 | 86028 | 19: 48709127 | 86032 | 19: 48880795 |
| 86042 | 19: 49040357 | 86051 | 19: 49233318 | 86055 | 19: 49277856 | 86063 | 19: 49478759 | 86064 | 19: 49483816 |
| 86065 | 19: 49494727 | 86068 | 19: 49575523 | 86076 | 19: 49772893 | 86078 | 19: 49810028 | 86085 | 19: 49891419 |
| 86092 | 19: 50063999 | 86094 | 19: 50095971 | 86095 | 19: 50107587 | 86096 | 19: 50123299 | 86099 | 19: 50167379 |
| 86100 | 19: 50172634 | 86101 | 19: 50173024 | 86102 | 19: 50173036 | 86103 | 19: 50173360 | 86104 | 19: 50173420 |
| 86116 | 19: 50607587 | 86118 | 19: 50662334 | 86122 | 19: 50682193 | 86139 | 19: 50783155 | 86140 | 19: 50784393 |
| 86142 | 19: 50788319 | 86144 | 19: 50790149 | 86145 | 19: 50790973 | 86146 | 19: 50791045 | 86147 | 19: 50817214 |
| 86148 | 19: 50817251 | 86150 | 19: 50838297 | 86151 | 19: 50838402 | 86152 | 19: 50855700 | 86153 | 19: 50856195 |
| 86154 | 19: 50883463 | 86155 | 19: 50890139 | 86156 | 19: 50892965 | 86157 | 19: 50893275 | 86158 | 19: 50893330 |
| 86159 | 19: 50893412 | 86160 | 19: 50894507 | 86164 | 19: 51146306 | 86165 | 19: 51148044 | 86166 | 19: 51150289 |
| 86167 | 19: 51151634 | 86168 | 19: 51152336 | 86169 | 19: 51152846 | 86170 | 19: 51153433 | 86171 | 19: 51155662 |
| 86172 | 19: 51156301 | 86173 | 19: 51158110 | 86174 | 19: 51172862 | 86175 | 19: 51173451 | 86176 | 19: 51179705 |
| 86177 | 19: 51181597 | 86178 | 19: 51184543 | 86179 | 19: 51185390 | 86180 | 19: 51186411 | 86181 | 19: 51186781 |
| 86182 | 19: 51186933 | 86183 | 19: 51187678 | 86184 | 19: 51192006 | 86185 | 19: 51194044 | 86186 | 19: 51195370 |
| 86187 | 19: 51196420 | 86188 | 19: 51197290 | 86189 | 19: 51197844 | 86190 | 19: 51198485 | 86191 | 19: 51200087 |
| 86192 | 19: 51202775 | 86193 | 19: 51203906 | 86194 | 19: 51204912 | 86195 | 19: 51206147 | 86196 | 19: 51206982 |
| 86197 | 19: 51208278 | 86198 | 19: 51209192 | 86199 | 19: 51215165 | 86213 | 19: 51584353 | 86214 | 19: 51614996 |
| 86215 | 19: 51624830 | 86219 | 19: 51657080 | 86220 | 19: 51674934 | 86221 | 19: 51674957 | 86222 | 19: 51674972 |
| 86223 | 19: 51676635 | 86225 | 19: 51688593 | 86226 | 19: 51689866 | 86238 | 19: 52025573 | 86243 | 19: 52232662 |
| 86246 | 19: 52242008 | 86261 | 19: 52716129 | 86275 | 19: 52957714 | 86277 | 19: 53006255 | 86279 | 19: 53041756 |
| 86281 | 19: 53068039 | 86285 | 19: 53125990 | 86286 | 19: 53132946 | 86301 | 19: 53433970 | 86302 | 19: 53439232 |
| 86304 | 19: 53484305 | 86308 | 19: 53566656 | 86309 | 19: 53572790 | 86310 | 19: 53588198 | 86314 | 19: 53698415 |
| 86318 | 19: 53748765 | 86320 | 19: 53774472 | 86321 | 19: 53778116 | 86323 | 19: 53811161 | 86333 | 19: 53948075 |
| 86337 | 19: 54028347 | 86338 | 19: 54043604 | 86341 | 19: 54053040 | 86352 | 19: 54289712 | 86353 | 19: 54295559 |
| 86355 | 19: 54301176 | 86358 | 19: 54390593 | 86371 | 19: 54622173 | 86372 | 19: 54637181 | 86379 | 19: 54710734 |
| 86383 | 19: 54755988 | 86388 | 19: 54858324 | 86393 | 19: 54978600 | 86398 | 19: 55092867 | 86403 | 19: 55229692 |
| 86406 | 19: 55265827 | 86409 | 19: 55330553 | 86413 | 19: 55427978 | 86414 | 19: 55428029 | 86422 | 19: 55580085 |
| 86427 | 19: 55698969 | 86432 | 19: 55792180 | 86433 | 19: 55793483 | 86434 | 19: 55793574 | 86436 | 19: 55827900 |
| 86437 | 19: 55830150 | 86440 | 19: 55848361 | 86441 | 19: 55848663 | 86444 | 19: 55866969 | 86449 | 19: 55906352 |
| 86452 | 19: 55911397 | 86454 | 19: 55915911 | 86455 | 19: 55917441 | 86456 | 19: 55920727 | 86458 | 19: 55922694 |
| 86462 | 19: 55926659 | 86464 | 19: 55940240 | 86471 | 19: 55985092 | 86472 | 19: 55985151 | 86473 | 19: 55985483 |
| 86474 | 19: 55986867 | 86475 | 19: 55986948 | 86477 | 19: 55987569 | 86479 | 19: 55988850 | 86480 | 19: 55988905 |
| 86481 | 19: 55988950 | 86482 | 19: 55989161 | 86483 | 19: 55989317 | 86484 | 19: 55989324 | 86486 | 19: 55993442 |
| 86489 | 19: 56009506 | 86495 | 19: 56024722 | 86500 | 19: 56044293 | 86503 | 19: 56059597 | 86512 | 19: 56100545 |
| 86516 | 19: 56104113 | 86535 | 19: 56318677 | 86536 | 19: 56320176 | 86537 | 19: 56346313 | 86538 | 19: 56346447 |
| 86539 | 19: 56348743 | 86540 | 19: 56388703 | 86544 | 19: 56447130 | 86549 | 19: 56504478 | 86550 | 19: 56506885 |
| 86551 | 19: 56506918 | 86553 | 19: 56521721 | 86554 | 19: 56527708 | 86556 | 19: 56536304 | 86558 | 19: 56542031 |
| 86559 | 19: 56544120 | 86560 | 19: 56545458 | 86561 | 19: 56546864 | 86562 | 19: 56553455 | 86571 | 19: 56792481 |
| 86573 | 19: 56794455 | 86576 | 19: 56891022 | 86579 | 19: 56962292 | 86581 | 19: 57011124 | 86582 | 19: 57012570 |
| 86591 | 19: 57196446 | 86592 | 19: 57214845 | 86593 | 19: 57259057 | 86600 | 19: 57418240 | 86608 | 19: 57566385 |
| 86631 | 19: 58091496 | 86632 | 19: 58091529 | 86634 | 19: 58127830 | 86638 | 19: 58214009 | 86639 | 19: 58218020 |
| 86645 | 19: 58304430 | 86650 | 19: 58429115 | 86659 | 19: 58594514 | 86665 | 19: 58750938 | 86673 | 19: 58918004 |
| 86688 | 19: 59274219 | 86689 | 19: 59281932 | 86711 | 19: 59592874 | 86718 | 19: 59660094 | 86724 | 19: 59743571 |
| 86727 | 19: 59784192 | 86731 | 19: 59871909 | 86732 | 19: 59872022 | 86742 | 19: 59984597 | 86756 | 19: 60173328 |
| 86768 | 19: 60319323 | 86776 | 19: 60416386 | 86781 | 19: 60493532 | 86782 | 19: 60495581 | 86793 | 19: 60643681 |
| 86795 | 19: 60660851 | 86800 | 19: 60743931 | 86802 | 19: 60816246 | 86811 | 19: 60977087 | 86814 | 19: 61065143 |
| 86818 | 19: 61145338 | 86819 | 19: 61146374 | 86822 | 19: 61175458 | 86823 | 19: 61175494 | 86824 | 19: 61175495 |
| 86825 | 19: 61175593 | 86826 | 19: 61175611 | 86834 | 19: 61242337 | 86845 | 19: 61459019 | 86846 | 19: 61474017 |
| 86848 | 19: 61529467 | 86849 | 19: 61531654 | 86850 | 19: 61531677 | 86851 | 19: 61532390 | 86852 | 19: 61533739 |
| 86854 | 19: 61538770 | 86862 | 19: 61639102 | 86865 | 19: 61681705 | 86867 | 19: 61701942 | 86868 | 19: 61709842 |
| 86874 | 19: 61794791 | 86884 | 19: 61956159 | 86885 | 19: 61968113 | 86892 | 19: 62135323 | 86904 | 19: 62322264 |
| 86915 | 19: 62611634 | 86916 | 19: 62620305 | 86919 | 19: 62738018 | 86927 | 19: 62774324 | 86928 | 19: 62785952 |
| 86942 | 19: 63013941 | 86944 | 19: 63088763 | 86946 | 19: 63091044 | 86959 | 19: 63394562 | 86960 | 19: 63394839 |
| 86965 | 20: 283185 | 86975 | 20: 495073 | 86979 | 20: 523765 | 86989 | 20: 666472 | 87009 | 20: 1104356 |
| 87012 | 20: 1164612 | 87016 | 20: 1365586 | 87019 | 20: 1476960 | 87020 | 20: 1477326 | 87025 | 20: 1585296 |
| 87031 | 20: 1627743 | 87032 | 20: 1627820 | 87033 | 20: 1628846 | 87038 | 20: 1708322 | 87040 | 20: 1708393 |
| 87051 | 20: 1881180 | 87054 | 20: 1890880 | 87058 | 20: 1964788 | 87059 | 20: 1981954 | 87060 | 20: 1985087 |
| 87062 | 20: 2008634 | 87066 | 20: 2111352 | 87071 | 20: 2174269 | 87082 | 20: 2329012 | 87084 | 20: 2363600 |
| 87090 | 20: 2486996 | 87093 | 20: 2549449 | 87094 | 20: 2549482 | 87123 | 20: 3122350 | 87125 | 20: 3132189 |
| 87126 | 20: 3144963 | 87127 | 20: 3147090 | 87128 | 20: 3149103 | 87133 | 20: 3256361 | 87141 | 20: 3434776 |
| 87143 | 20: 3442837 | 87144 | 20: 3490573 | 87153 | 20: 3643964 | 87163 | 20: 3970013 | 87184 | 20: 4419380 |
| 87186 | 20: 4422606 | 87187 | 20: 4422634 | 87189 | 20: 4471095 | 87201 | 20: 4600219 | 87353 | 20: 4974566 |
| 87356 | 20: 4978213 | 87396 | 20: 5081279 | 87397 | 20: 5081368 | 87398 | 20: 5081429 | 87411 | 20: 5256314 |
| 87424 | 20: 5427984 | 87425 | 20: 5449185 | 87427 | 20: 5456791 | 87445 | 20: 5952561 | 87471 | 20: 6124117 |
| 87472 | 20: 6127051 | 87473 | 20: 6128007 | 87474 | 20: 6129222 | 87475 | 20: 6135444 | 87478 | 20: 6222819 |
| 87491 | 20: 6538447 | 87493 | 20: 6552399 | 87502 | 20: 6787304 | 87503 | 20: 6803666 | 87504 | 20: 6804954 |
| 87505 | 20: 6809070 | 87507 | 20: 6819134 | 87508 | 20: 6824958 | 87514 | 20: 7026132 | 87515 | 20: 7028111 |
| 87527 | 20: 7184948 | 87529 | 20: 7203346 | 87530 | 20: 7228830 | 87536 | 20: 7397715 | 87538 | 20: 7452989 |
| 87556 | 20: 7937085 | 87558 | 20: 7966207 | 87565 | 20: 8099607 | 87580 | 20: 8406030 | 87588 | 20: 8596907 |
| 87591 | 20: 8676605 | 87597 | 20: 8915848 | 87599 | 20: 8976316 | 87600 | 20: 8994477 | 87603 | 20: 9008027 |
| 87604 | 20: 9008070 | 87610 | 20: 9122965 | 87611 | 20: 9185096 | 87616 | 20: 9277064 | 87617 | 20: 9284840 |
| 87618 | 20: 9286211 | 87619 | 20: 9286666 | 87620 | 20: 9297979 | 87622 | 20: 9309443 | 87627 | 20: 9387110 |
| 87635 | 20: 9562025 | 87642 | 20: 9678407 | 87643 | 20: 9699294 | 87653 | 20: 9987753 | 87657 | 20: 10105910 |
| 87670 | 20: 10568285 | 87677 | 20: 10719241 | 87691 | 20: 11104981 | 87695 | 20: 11173739 | 87708 | 20: 11384109 |
| 87709 | 20: 11423657 | 87712 | 20: 11441654 | 87719 | 20: 11603501 | 87723 | 20: 11652136 | 87726 | 20: 11746445 |
| 87727 | 20: 11748411 | 87729 | 20: 11801270 | 87732 | 20: 11873589 | 87736 | 20: 12001911 | 87745 | 20: 12195219 |
| 87751 | 20: 12336592 | 87756 | 20: 12431016 | 87759 | 20: 12454756 | 87760 | 20: 12470759 | 87770 | 20: 12619125 |
| 87776 | 20: 12725086 | 87777 | 20: 12732314 | 87787 | 20: 12872002 | 87788 | 20: 12884968 | 87792 | 20: 13038217 |
| 87800 | 20: 13212537 | 87804 | 20: 13314745 | 87806 | 20: 13360616 | 87808 | 20: 13384250 | 87809 | 20: 13411239 |
| 87820 | 20: 13846644 | 87821 | 20: 13853397 | 87827 | 20: 13967913 | 87881 | 20: 16013654 | 87890 | 20: 16311925 |
| 87891 | 20: 16312024 | 87892 | 20: 16312059 | 87893 | 20: 16312062 | 87926 | 20: 17161518 | 87940 | 20: 17529786 |
| 87946 | 20: 17718360 | 87949 | 20: 17740634 | 87950 | 20: 17772296 | 87959 | 20: 18106764 | 87966 | 20: 18199987 |
| 87974 | 20: 18560092 | 87979 | 20: 18738258 | 87981 | 20: 18767430 | 87988 | 20: 18999163 | 87998 | 20: 19219710 |
| 88010 | 20: 19525255 | 88013 | 20: 19638381 | 88024 | 20: 19910542 | 88026 | 20: 19934333 | 88029 | 20: 19951507 |
| 88030 | 20: 19985004 | 88035 | 20: 20229781 | 88049 | 20: 20735095 | 88053 | 20: 20833870 | 88054 | 20: 20847713 |
| 88055 | 20: 20852713 | 88057 | 20: 20900999 | 88058 | 20: 20916403 | 88060 | 20: 20932871 | 88062 | 20: 20985150 |
| 88069 | 20: 21165648 | 88070 | 20: 21199073 | 88076 | 20: 21442552 | 88119 | 20: 22069390 | 88123 | 20: 22130295 |
| 88158 | 20: 22204908 | 88186 | 20: 22465821 | 88204 | 20: 22827690 | 88213 | 20: 23009076 | 88214 | 20: 23038319 |
| 88215 | 20: 23039137 | 88224 | 20: 23272824 | 88234 | 20: 23534898 | 88236 | 20: 23571173 | 88237 | 20: 23574812 |
| 88238 | 20: 23575304 | 88242 | 20: 23686099 | 88243 | 20: 23692874 | 88248 | 20: 23836890 | 88249 | 20: 23837832 |
| 88250 | 20: 23838131 | 88251 | 20: 23838181 | 88252 | 20: 23841545 | 88253 | 20: 23861537 | 88254 | 20: 23861790 |
| 88255 | 20: 23862521 | 88256 | 20: 23863171 | 88257 | 20: 23864163 | 88258 | 20: 23864320 | 88260 | 20: 23868037 |
| 88261 | 20: 23872171 | 88262 | 20: 23872177 | 88263 | 20: 23873363 | 88265 | 20: 23931936 | 88266 | 20: 23934587 |
| 88267 | 20: 23936814 | 88268 | 20: 23937494 | 88269 | 20: 23940208 | 88270 | 20: 23940805 | 88271 | 20: 23948706 |
| 88272 | 20: 23950019 | 88273 | 20: 23956567 | 88274 | 20: 23967747 | 88278 | 20: 24005106 | 88283 | 20: 24183792 |
| 88289 | 20: 24300670 | 88291 | 20: 24342520 | 88301 | 20: 24551449 | 88303 | 20: 24600379 | 88304 | 20: 24644625 |
| 88314 | 20: 24840568 | 88316 | 20: 24888410 | 88336 | 20: 25687633 | 88341 | 20: 25956846 | 88353 | 20: 26326568 |
| 88377 | 20: 26904319 | 88394 | 20: 27389017 | 88396 | 20: 27389119 | 88418 | 20: 27942740 | 88423 | 20: 28062228 |
| 88424 | 20: 28062230 | 88425 | 20: 28067347 | 88435 | 20: 28291529 | 88440 | 20: 28407293 | 88449 | 20: 28594465 |
| 88452 | 20: 28714693 | 88453 | 20: 28719941 | 88454 | 20: 28719958 | 88455 | 20: 28720052 | 88456 | 20: 28726509 |
| 88457 | 20: 28726541 | 88458 | 20: 28726553 | 88459 | 20: 28726570 | 88460 | 20: 28726626 | 88468 | 20: 28835792 |
| 88474 | 20: 28977237 | 88475 | 20: 28978238 | 88488 | 20: 29362772 | 88489 | 20: 29376013 | 88491 | 20: 29447835 |
| 88508 | 20: 29990492 | 88509 | 20: 29990621 | 88514 | 20: 30118413 | 88518 | 20: 30200252 | 88520 | 20: 30212011 |
| 88532 | 20: 30669895 | 88536 | 20: 30689871 | 88539 | 20: 30825518 | 88546 | 20: 31205981 | 88547 | 20: 31211377 |
| 88548 | 20: 31243755 | 88549 | 20: 31245339 | 88558 | 20: 31456913 | 88567 | 20: 31684137 | 88570 | 20: 31752397 |
| 88576 | 20: 31881419 | 88579 | 20: 31903713 | 88582 | 20: 31972125 | 88585 | 20: 32052139 | 88588 | 20: 32101132 |
| 88597 | 20: 32409103 | 88633 | 20: 33398481 | 88637 | 20: 33502768 | 88640 | 20: 33565104 | 88641 | 20: 33569466 |
| 88648 | 20: 33719114 | 88653 | 20: 33836807 | 88657 | 20: 34001523 | 88663 | 20: 34145466 | 88666 | 20: 34184342 |
| 88669 | 20: 34272037 | 88692 | 20: 34738766 | 88694 | 20: 34781170 | 88697 | 20: 34913079 | 88714 | 20: 35421048 |
| 88721 | 20: 35551560 | 88722 | 20: 35585029 | 88723 | 20: 35610755 | 88726 | 20: 35701545 | 88728 | 20: 35715693 |
| 88736 | 20: 35846193 | 88737 | 20: 35882733 | 88741 | 20: 35994055 | 88742 | 20: 35994063 | 88743 | 20: 35996784 |
| 88762 | 20: 36319426 | 88767 | 20: 36496636 | 88776 | 20: 36648116 | 88781 | 20: 36787045 | 88782 | 20: 36834159 |
| 88783 | 20: 36858954 | 88785 | 20: 36892081 | 88788 | 20: 36968962 | 88789 | 20: 36989703 | 88791 | 20: 37042637 |
| 88794 | 20: 37085325 | 88795 | 20: 37092573 | 88797 | 20: 37131660 | 88798 | 20: 37131690 | 88799 | 20: 37136413 |
| 88804 | 20: 37341795 | 88810 | 20: 37443371 | 88811 | 20: 37443444 | 88812 | 20: 37443481 | 88813 | 20: 37473672 |
| 88815 | 20: 37479797 | 88816 | 20: 37481956 | 88827 | 20: 37694264 | 88828 | 20: 37694333 | 88831 | 20: 37811388 |
| 88832 | 20: 37811427 | 88833 | 20: 37811480 | 88834 | 20: 37811543 | 88838 | 20: 37887781 | 88845 | 20: 38177941 |
| 88846 | 20: 38189067 | 88867 | 20: 38758576 | 88881 | 20: 39088930 | 88882 | 20: 39109157 | 88887 | 20: 39238537 |
| 88888 | 20: 39245057 | 88890 | 20: 39287657 | 88893 | 20: 39383171 | 88894 | 20: 39383264 | 88898 | 20: 39445232 |
| 88904 | 20: 39475943 | 88909 | 20: 39539960 | 88911 | 20: 39555296 | 88912 | 20: 39557649 | 88924 | 20: 39810901 |
| 88925 | 20: 39863071 | 88928 | 20: 39910970 | 88930 | 20: 39965197 | 88937 | 20: 40059831 | 88941 | 20: 40215915 |
| 88955 | 20: 40683858 | 88956 | 20: 40684807 | 88957 | 20: 40695016 | 88961 | 20: 40772462 | 88966 | 20: 40834506 |
| 88968 | 20: 40945725 | 88975 | 20: 41050907 | 88976 | 20: 41050966 | 88978 | 20: 41100712 | 88984 | 20: 41160179 |
| 88986 | 20: 41192863 | 88990 | 20: 41314439 | 89010 | 20: 41798915 | 89012 | 20: 41819893 | 89013 | 20: 41823837 |
| 89016 | 20: 41845476 | 89023 | 20: 41919178 | 89028 | 20: 42044095 | 89029 | 20: 42045334 | 89032 | 20: 42085412 |
| 89035 | 20: 42133834 | 89039 | 20: 42246244 | 89042 | 20: 42293890 | 89043 | 20: 42331127 | 89047 | 20: 42565028 |
| 89061 | 20: 42942479 | 89066 | 20: 43159533 | 89079 | 20: 43561846 | 89080 | 20: 43561856 | 89101 | 20: 44025947 |
| 89102 | 20: 44026070 | 89107 | 20: 44281699 | 89108 | 20: 44301844 | 89121 | 20: 44556774 | 89122 | 20: 44574229 |
| 89123 | 20: 44591612 | 89133 | 20: 44966449 | 89134 | 20: 44969446 | 89145 | 20: 45365363 | 89186 | 20: 46291117 |
| 89194 | 20: 46578492 | 89208 | 20: 47160581 | 89209 | 20: 47160583 | 89211 | 20: 47163202 | 89212 | 20: 47163219 |
| 89243 | 20: 48405434 | 89244 | 20: 48405481 | 89245 | 20: 48405501 | 89246 | 20: 48405505 | 89247 | 20: 48405507 |
| 89248 | 20: 48405524 | 89249 | 20: 48405575 | 89251 | 20: 48474578 | 89262 | 20: 48874528 | 89264 | 20: 48881242 |
| 89265 | 20: 48881303 | 89266 | 20: 48881343 | 89267 | 20: 48901928 | 89274 | 20: 49054367 | 89297 | 20: 49861661 |
| 89298 | 20: 49913380 | 89299 | 20: 49915377 | 89300 | 20: 49918452 | 89323 | 20: 50694458 | 89328 | 20: 50806661 |
| 89338 | 20: 51034291 | 89363 | 20: 51929370 | 89385 | 20: 52736752 | 89394 | 20: 52981985 | 89401 | 20: 53204896 |
| 89405 | 20: 53347849 | 89415 | 20: 53521298 | 89416 | 20: 53526124 | 89420 | 20: 53570726 | 89428 | 20: 53840361 |
| 89442 | 20: 54395462 | 89465 | 20: 55054760 | 89466 | 20: 55055902 | 89478 | 20: 55217698 | 89485 | 20: 55403499 |
| 89500 | 20: 55900412 | 89503 | 20: 55938086 | 89522 | 20: 56361024 | 89526 | 20: 56443806 | 89530 | 20: 56569222 |
| 89532 | 20: 56609894 | 89533 | 20: 56609977 | 89536 | 20: 56692099 | 89541 | 20: 56762906 | 89570 | 20: 57486934 |
| 89571 | 20: 57487439 | 89576 | 20: 57572314 | 89584 | 20: 57778974 | 89595 | 20: 57998700 | 89596 | 20: 57998708 |
| 89597 | 20: 57998777 | 89598 | 20: 57998802 | 89599 | 20: 57998807 | 89600 | 20: 57998808 | 89601 | 20: 57998848 |
| 89602 | 20: 57998909 | 89604 | 20: 58038602 | 89605 | 20: 58038638 | 89606 | 20: 58038676 | 89610 | 20: 58171888 |
| 89622 | 20: 58370776 | 89623 | 20: 58371818 | 89628 | 20: 58425428 | 89630 | 20: 58432597 | 89638 | 20: 58662715 |
| 89652 | 20: 58839976 | 89653 | 20: 58841417 | 89661 | 20: 58985542 | 89665 | 20: 59056598 | 89668 | 20: 59062072 |
| 89675 | 20: 59262668 | 89696 | 20: 59893295 | 89698 | 20: 59918108 | 89699 | 20: 59918158 | 89700 | 20: 59918229 |
| 89702 | 20: 59984000 | 89750 | 20: 61610794 | 89751 | 20: 61610854 | 89755 | 20: 61693804 | 89767 | 20: 61962563 |
| 89773 | 20: 62086784 | 89786 | 20: 62394644 | 89790 | 20: 62513410 | 89792 | 20: 62528844 | 89794 | 20: 62575332 |
| 89799 | 20: 62653294 | 89800 | 20: 62670652 | 89808 | 20: 62848870 | 89810 | 20: 62914219 | 89811 | 20: 62937049 |
| 89812 | 20: 62942539 | 89813 | 20: 62963027 | 89815 | 20: 62971781 | 89828 | 20: 63314880 | 89830 | 20: 63382028 |
| 89831 | 20: 63384182 | 89841 | 20: 63652842 | 89843 | 20: 63711747 | 89848 | 20: 63813002 | 89857 | 20: 63923149 |
| 89858 | 20: 63934159 | 89859 | 20: 63939074 | 89860 | 20: 63939894 | 89861 | 20: 63949508 | 89862 | 20: 63950751 |
| 89863 | 20: 63952037 | 89864 | 20: 63952862 | 89865 | 20: 63954238 | 89866 | 20: 63981759 | 89871 | 20: 64108375 |
| 89891 | 20: 64483792 | 89912 | 20: 65101244 | 89919 | 20: 65253620 | 89927 | 20: 65566194 | 89928 | 20: 65578450 |
| 89929 | 20: 65592431 | 89932 | 20: 65619064 | 89934 | 20: 65691397 | 89936 | 20: 65761375 | 89937 | 20: 65773012 |
| 89938 | 20: 65786774 | 89945 | 20: 65973669 | 89950 | 20: 66089403 | 89955 | 20: 66319547 | 89959 | 20: 66370625 |
| 89960 | 20: 66372293 | 89962 | 20: 66457113 | 89963 | 20: 66464862 | 89964 | 20: 66468697 | 89967 | 20: 66551247 |
| 89968 | 20: 66556459 | 89969 | 20: 66563281 | 89970 | 20: 66563428 | 89971 | 20: 66567783 | 89972 | 20: 66568233 |
| 89973 | 20: 66626549 | 89976 | 20: 66691115 | 89978 | 20: 66696604 | 89979 | 20: 66711575 | 89980 | 20: 66711602 |
| 90001 | 20: 67153888 | 90012 | 20: 67465264 | 90016 | 20: 67521067 | 90026 | 20: 67735557 | 90027 | 20: 67739397 |
| 90028 | 20: 67743005 | 90031 | 20: 67808126 | 90032 | 20: 67819595 | 90035 | 20: 67872456 | 90039 | 20: 67995355 |
| 90041 | 20: 68014834 | 90054 | 20: 68168592 | 90062 | 20: 68323418 | 90063 | 20: 68352541 | 90071 | 20: 68463408 |
| 90072 | 20: 68503575 | 90079 | 20: 68640146 | 90087 | 20: 68746121 | 90095 | 20: 68908716 | 90102 | 20: 68990075 |
| 90129 | 20: 69518490 | 90157 | 20: 70210220 | 90182 | 20: 70941731 | 90183 | 20: 70948562 | 90184 | 20: 70949203 |
| 90185 | 20: 70954723 | 90187 | 20: 70972905 | 90188 | 20: 70973702 | 90189 | 20: 70975538 | 90193 | 20: 71082114 |
| 90194 | 20: 71097489 | 90195 | 20: 71099307 | 90196 | 20: 71104986 | 90197 | 20: 71108601 | 90198 | 20: 71110857 |
| 90199 | 20: 71113969 | 90200 | 20: 71118893 | 90201 | 20: 71119829 | 90202 | 20: 71121420 | 90203 | 20: 71122291 |
| 90204 | 20: 71124183 | 90205 | 20: 71124516 | 90206 | 20: 71127650 | 90207 | 20: 71131787 | 90208 | 20: 71139430 |
| 90209 | 20: 71141395 | 90210 | 20: 71147142 | 90211 | 20: 71147843 | 90212 | 20: 71150904 | 90213 | 20: 71153153 |
| 90214 | 20: 71154456 | 90215 | 20: 71162376 | 90216 | 20: 71172637 | 90217 | 20: 71173872 | 90218 | 20: 71177834 |
| 90219 | 20: 71180101 | 90220 | 20: 71181518 | 90221 | 20: 71183377 | 90222 | 20: 71188295 | 90223 | 20: 71195927 |
| 90224 | 20: 71201866 | 90225 | 20: 71203976 | 90226 | 20: 71206269 | 90227 | 20: 71207699 | 90228 | 20: 71212077 |
| 90229 | 20: 71232016 | 90230 | 20: 71240516 | 90231 | 20: 71290453 | 90232 | 20: 71293773 | 90233 | 20: 71295284 |
| 90234 | 20: 71300630 | 90235 | 20: 71301433 | 90236 | 20: 71313672 | 90237 | 20: 71319974 | 90238 | 20: 71320628 |
| 90239 | 20: 71349322 | 90240 | 20: 71354558 | 90241 | 20: 71355354 | 90242 | 20: 71359918 | 90243 | 20: 71360768 |
| 90244 | 20: 71378946 | 90245 | 20: 71385619 | 90246 | 20: 71386554 | 90247 | 20: 71387739 | 90248 | 20: 71388363 |
| 90249 | 20: 71393864 | 90250 | 20: 71395443 | 90251 | 20: 71396187 | 90252 | 20: 71401124 | 90254 | 20: 71402777 |
| 90255 | 20: 71403559 | 90256 | 20: 71407893 | 90257 | 20: 71417623 | 90258 | 20: 71431631 | 90259 | 20: 71442260 |
| 90260 | 20: 71444098 | 90261 | 20: 71447274 | 90262 | 20: 71448297 | 90263 | 20: 71448849 | 90264 | 20: 71450177 |
| 90265 | 20: 71450861 | 90266 | 20: 71459002 | 90267 | 20: 71472317 | 90268 | 20: 71490172 | 90269 | 20: 71491300 |
| 90272 | 20: 71606988 | 90280 | 20: 71927868 | 90305 | 21: 1606872 | 90320 | 21: 2377105 | 90330 | 21: 2684106 |
| 90336 | 21: 2874100 | 90344 | 21: 3001448 | 90366 | 21: 3866539 | 90375 | 21: 4053839 | 90377 | 21: 4055731 |
| 90378 | 21: 4060826 | 90402 | 21: 4420712 | 90413 | 21: 4948777 | 90420 | 21: 5153009 | 90425 | 21: 5327112 |
| 90428 | 21: 5382657 | 90436 | 21: 5552825 | 90437 | 21: 5575472 | 90441 | 21: 5776117 | 90449 | 21: 5865604 |
| 90453 | 21: 5946488 | 90454 | 21: 5946554 | 90455 | 21: 5961487 | 90460 | 21: 6096270 | 90469 | 21: 6466492 |
| 90471 | 21: 6515336 | 90477 | 21: 6718976 | 90479 | 21: 6821186 | 90480 | 21: 6838984 | 90481 | 21: 6864243 |
| 90488 | 21: 7110184 | 90489 | 21: 7126782 | 90506 | 21: 7582228 | 90508 | 21: 7589812 | 90509 | 21: 7590462 |
| 90520 | 21: 7863440 | 90521 | 21: 7868053 | 90524 | 21: 7907745 | 90530 | 21: 8033587 | 90534 | 21: 8074100 |
| 90535 | 21: 8074643 | 90552 | 21: 8176568 | 90554 | 21: 8190635 | 90555 | 21: 8226423 | 90562 | 21: 8310287 |
| 90604 | 21: 9465633 | 90628 | 21: 9948207 | 90632 | 21: 10010929 | 90642 | 21: 10296960 | 90647 | 21: 10509482 |
| 90653 | 21: 10609946 | 90654 | 21: 10624234 | 90671 | 21: 10957495 | 90679 | 21: 11069313 | 90693 | 21: 11440159 |
| 90694 | 21: 11445014 | 90698 | 21: 11556518 | 90703 | 21: 11654700 | 90743 | 21: 12681133 | 90749 | 21: 12760689 |
| 90754 | 21: 12855624 | 90776 | 21: 13353466 | 90780 | 21: 13514487 | 90794 | 21: 13810003 | 90798 | 21: 13905626 |
| 90805 | 21: 14029521 | 90811 | 21: 14202552 | 90823 | 21: 14517580 | 90824 | 21: 14560006 | 90834 | 21: 14713608 |
| 90840 | 21: 14945241 | 90848 | 21: 15132400 | 90849 | 21: 15138571 | 90851 | 21: 15156831 | 90852 | 21: 15156885 |
| 90853 | 21: 15156911 | 90856 | 21: 15219073 | 90859 | 21: 15326382 | 90888 | 21: 15932209 | 90894 | 21: 16294354 |
| 90896 | 21: 16311821 | 90897 | 21: 16324443 | 90901 | 21: 16376470 | 90905 | 21: 16407620 | 90906 | 21: 16426000 |
| 90908 | 21: 16455704 | 90912 | 21: 16482563 | 90914 | 21: 16507141 | 90919 | 21: 16581532 | 90920 | 21: 16583122 |
| 90922 | 21: 16605546 | 90930 | 21: 16771714 | 90932 | 21: 16775550 | 90933 | 21: 16780485 | 90940 | 21: 16994759 |
| 90945 | 21: 17105820 | 90947 | 21: 17157614 | 90967 | 21: 17696116 | 91010 | 21: 18775346 | 91011 | 21: 18792766 |
| 91012 | 21: 18796836 | 91013 | 21: 18806197 | 91014 | 21: 18807592 | 91015 | 21: 18807654 | 91018 | 21: 18889130 |
| 91019 | 21: 18889216 | 91020 | 21: 18889225 | 91021 | 21: 18897548 | 91022 | 21: 18897617 | 91023 | 21: 18897699 |
| 91024 | 21: 18906129 | 91025 | 21: 18906131 | 91035 | 21: 19086207 | 91044 | 21: 19274749 | 91045 | 21: 19274809 |
| 91049 | 21: 19328532 | 91050 | 21: 19341878 | 91058 | 21: 19448733 | 91059 | 21: 19484240 | 91061 | 21: 19509806 |
| 91065 | 21: 19547342 | 91066 | 21: 19566627 | 91067 | 21: 19571760 | 91068 | 21: 19574086 | 91069 | 21: 19574861 |
| 91071 | 21: 19581384 | 91072 | 21: 19587859 | 91074 | 21: 19591585 | 91077 | 21: 19636076 | 91078 | 21: 19650309 |
| 91079 | 21: 19744871 | 91080 | 21: 19745043 | 91081 | 21: 19765456 | 91082 | 21: 19765736 | 91083 | 21: 19765864 |
| 91084 | 21: 19765895 | 91085 | 21: 19765940 | 91088 | 21: 19791905 | 91089 | 21: 19796807 | 91090 | 21: 19800176 |
| 91092 | 21: 19819891 | 91093 | 21: 19837076 | 91120 | 21: 20576639 | 91123 | 21: 20597014 | 91124 | 21: 20611156 |
| 91129 | 21: 20643657 | 91132 | 21: 20661675 | 91137 | 21: 20707916 | 91139 | 21: 20756163 | 91140 | 21: 20767918 |
| 91148 | 21: 20994233 | 91149 | 21: 20997368 | 91150 | 21: 21002238 | 91153 | 21: 21051410 | 91154 | 21: 21051969 |
| 91155 | 21: 21079825 | 91173 | 21: 21544579 | 91175 | 21: 21547758 | 91176 | 21: 21554110 | 91178 | 21: 21572136 |
| 91189 | 21: 21882815 | 91195 | 21: 21997010 | 91197 | 21: 22018834 | 91201 | 21: 22122091 | 91202 | 21: 22130826 |
| 91203 | 21: 22132465 | 91204 | 21: 22137495 | 91205 | 21: 22138799 | 91208 | 21: 22168604 | 91217 | 21: 22354151 |
| 91222 | 21: 22473177 | 91223 | 21: 22478617 | 91232 | 21: 22695822 | 91234 | 21: 22803320 | 91240 | 21: 22922296 |
| 91244 | 21: 22980503 | 91247 | 21: 23023062 | 91249 | 21: 23081635 | 91250 | 21: 23092163 | 91254 | 21: 23177845 |
| 91255 | 21: 23177853 | 91256 | 21: 23181041 | 91257 | 21: 23190056 | 91258 | 21: 23190653 | 91259 | 21: 23195238 |
| 91260 | 21: 23211467 | 91284 | 21: 24046484 | 91288 | 21: 24273010 | 91293 | 21: 24375735 | 91347 | 21: 26116827 |
| 91361 | 21: 26448325 | 91368 | 21: 26581951 | 91371 | 21: 26637918 | 91383 | 21: 26979057 | 91384 | 21: 26999182 |
| 91385 | 21: 26999251 | 91397 | 21: 27503033 | 91398 | 21: 27508911 | 91407 | 21: 27743344 | 91427 | 21: 28338007 |
| 91441 | 21: 28917960 | 91443 | 21: 28948600 | 91444 | 21: 28961889 | 91448 | 21: 29052667 | 91461 | 21: 29439618 |
| 91465 | 21: 29553919 | 91471 | 21: 29725150 | 91488 | 21: 30205371 | 91489 | 21: 30225427 | 91493 | 21: 30359230 |
| 91494 | 21: 30364775 | 91495 | 21: 30364784 | 91497 | 21: 30380463 | 91498 | 21: 30383546 | 91500 | 21: 30415216 |
| 91504 | 21: 30548247 | 91507 | 21: 30574539 | 91508 | 21: 30583259 | 91512 | 21: 30655015 | 91513 | 21: 30665920 |
| 91514 | 21: 30666267 | 91518 | 21: 30752337 | 91521 | 21: 30836177 | 91525 | 21: 31068352 | 91526 | 21: 31085819 |
| 91528 | 21: 31169053 | 91538 | 21: 31453639 | 91539 | 21: 31464548 | 91540 | 21: 31470928 | 91541 | 21: 31485138 |
| 91542 | 21: 31493718 | 91543 | 21: 31496586 | 91544 | 21: 31499460 | 91545 | 21: 31522156 | 91546 | 21: 31525621 |
| 91547 | 21: 31534000 | 91548 | 21: 31541459 | 91549 | 21: 31545947 | 91550 | 21: 31553803 | 91551 | 21: 31604982 |
| 91553 | 21: 31720553 | 91554 | 21: 31725673 | 91555 | 21: 31734161 | 91556 | 21: 31736971 | 91557 | 21: 31754761 |
| 91558 | 21: 31758189 | 91559 | 21: 31781060 | 91560 | 21: 31791229 | 91563 | 21: 31955982 | 91574 | 21: 32336068 |
| 91575 | 21: 32364556 | 91578 | 21: 32403515 | 91584 | 21: 32692535 | 91585 | 21: 32705474 | 91589 | 21: 32783989 |
| 91590 | 21: 32814280 | 91598 | 21: 33110998 | 91602 | 21: 33166758 | 91603 | 21: 33168203 | 91604 | 21: 33169028 |
| 91605 | 21: 33170204 | 91606 | 21: 33170309 | 91607 | 21: 33171324 | 91608 | 21: 33171538 | 91610 | 21: 33205680 |
| 91611 | 21: 33205893 | 91612 | 21: 33208660 | 91614 | 21: 33215961 | 91615 | 21: 33225284 | 91622 | 21: 33373120 |
| 91623 | 21: 33373124 | 91627 | 21: 33449736 | 91628 | 21: 33458505 | 91629 | 21: 33459344 | 91630 | 21: 33461031 |
| 91631 | 21: 33461192 | 91632 | 21: 33461449 | 91633 | 21: 33465757 | 91634 | 21: 33465758 | 91635 | 21: 33466480 |
| 91636 | 21: 33469426 | 91637 | 21: 33469555 | 91638 | 21: 33469624 | 91639 | 21: 33469729 | 91640 | 21: 33469770 |
| 91641 | 21: 33469982 | 91642 | 21: 33483461 | 91643 | 21: 33487373 | 91645 | 21: 33529381 | 91648 | 21: 33547120 |
| 91650 | 21: 33571261 | 91652 | 21: 33652175 | 91654 | 21: 33661162 | 91670 | 21: 33947582 | 91672 | 21: 33991645 |
| 91675 | 21: 34024711 | 91676 | 21: 34044738 | 91677 | 21: 34049707 | 91680 | 21: 34087450 | 91691 | 21: 34273652 |
| 91692 | 21: 34276064 | 91694 | 21: 34296572 | 91703 | 21: 34434123 | 91705 | 21: 34478879 | 91706 | 21: 34487765 |
| 91707 | 21: 34490950 | 91712 | 21: 34556901 | 91713 | 21: 34556925 | 91714 | 21: 34557109 | 91715 | 21: 34585611 |
| 91718 | 21: 34637208 | 91719 | 21: 34637260 | 91731 | 21: 34938997 | 91732 | 21: 34939108 | 91737 | 21: 35075467 |
| 91741 | 21: 35177228 | 91742 | 21: 35177300 | 91752 | 21: 35387396 | 91753 | 21: 35391211 | 91755 | 21: 35545267 |
| 91761 | 21: 35621906 | 91787 | 21: 36004086 | 91801 | 21: 36349981 | 91807 | 21: 36467140 | 91844 | 21: 37588472 |
| 91855 | 21: 37884949 | 91856 | 21: 37889952 | 91863 | 21: 38352155 | 91866 | 21: 38488688 | 91901 | 21: 39497698 |
| 91902 | 21: 39497796 | 91927 | 21: 40117870 | 91928 | 21: 40123103 | 91930 | 21: 40267167 | 91934 | 21: 40321474 |
| 91950 | 21: 40777585 | 91955 | 21: 41031761 | 91956 | 21: 41063587 | 91958 | 21: 41103681 | 91959 | 21: 41189588 |
| 91960 | 21: 41190356 | 91961 | 21: 41230647 | 91963 | 21: 41268397 | 91968 | 21: 41377730 | 91969 | 21: 41377811 |
| 91971 | 21: 41381520 | 91972 | 21: 41381521 | 91973 | 21: 41381562 | 91979 | 21: 41500659 | 91980 | 21: 41500733 |
| 91988 | 21: 41683205 | 91995 | 21: 41951773 | 91999 | 21: 41985647 | 92001 | 21: 42021205 | 92005 | 21: 42056585 |
| 92006 | 21: 42056665 | 92011 | 21: 42193358 | 92015 | 21: 42263974 | 92025 | 21: 42613095 | 92026 | 21: 42613704 |
| 92027 | 21: 42615428 | 92028 | 21: 42632006 | 92036 | 21: 42789940 | 92037 | 21: 42789959 | 92038 | 21: 42794024 |
| 92042 | 21: 42894892 | 92043 | 21: 42894971 | 92044 | 21: 42906559 | 92048 | 21: 42991321 | 92060 | 21: 43369442 |
| 92073 | 21: 43694807 | 92090 | 21: 44134802 | 92093 | 21: 44170697 | 92094 | 21: 44193483 | 92095 | 21: 44198391 |
| 92096 | 21: 44204274 | 92097 | 21: 44204999 | 92098 | 21: 44208959 | 92103 | 21: 44374543 | 92108 | 21: 44464711 |
| 92111 | 21: 44510750 | 92112 | 21: 44558742 | 92119 | 21: 44670112 | 92124 | 21: 44711056 | 92125 | 21: 44711119 |
| 92128 | 21: 44747742 | 92131 | 21: 44794407 | 92137 | 21: 44874950 | 92146 | 21: 45364186 | 92153 | 21: 45617906 |
| 92162 | 21: 45840926 | 92168 | 21: 46169637 | 92177 | 21: 46371720 | 92178 | 21: 46372978 | 92179 | 21: 46375756 |
| 92180 | 21: 46377528 | 92181 | 21: 46378483 | 92182 | 21: 46399567 | 92183 | 21: 46411679 | 92207 | 21: 47030345 |
| 92209 | 21: 47120773 | 92210 | 21: 47135961 | 92235 | 21: 47300254 | 92252 | 21: 47315681 | 92255 | 21: 47399940 |
| 92260 | 21: 47464301 | 92272 | 21: 47936288 | 92293 | 21: 48482014 | 92308 | 21: 48897167 | 92315 | 21: 49088678 |
| 92316 | 21: 49165291 | 92317 | 21: 49165372 | 92325 | 21: 49212043 | 92348 | 21: 49756597 | 92349 | 21: 49773549 |
| 92414 | 21: 51757967 | 92423 | 21: 52002988 | 92430 | 21: 52140964 | 92438 | 21: 52391191 | 92447 | 21: 52590908 |
| 92459 | 21: 53100162 | 92467 | 21: 53274187 | 92468 | 21: 53295524 | 92469 | 21: 53295551 | 92473 | 21: 53382594 |
| 92494 | 21: 53955771 | 92511 | 21: 54486141 | 92519 | 21: 54707788 | 92533 | 21: 54895636 | 92534 | 21: 54906059 |
| 92535 | 21: 54910848 | 92536 | 21: 54911588 | 92537 | 21: 54911598 | 92538 | 21: 54919544 | 92540 | 21: 54956256 |
| 92544 | 21: 55027058 | 92549 | 21: 55139938 | 92551 | 21: 55157787 | 92553 | 21: 55268142 | 92555 | 21: 55276126 |
| 92559 | 21: 55336100 | 92567 | 21: 55534889 | 92600 | 21: 56030046 | 92609 | 21: 56102239 | 92611 | 21: 56151393 |
| 92612 | 21: 56165597 | 92613 | 21: 56174882 | 92614 | 21: 56184939 | 92616 | 21: 56194199 | 92617 | 21: 56195714 |
| 92618 | 21: 56211969 | 92625 | 21: 56552387 | 92627 | 21: 56570919 | 92637 | 21: 56804066 | 92689 | 21: 57070861 |
| 92690 | 21: 57070865 | 92713 | 21: 57163461 | 92714 | 21: 57163519 | 92730 | 21: 57357698 | 92737 | 21: 57514823 |
| 92740 | 21: 57650013 | 92744 | 21: 57789826 | 92745 | 21: 57815184 | 92746 | 21: 57841247 | 92752 | 21: 58086488 |
| 92760 | 21: 58328406 | 92761 | 21: 58335169 | 92762 | 21: 58348854 | 92765 | 21: 58457450 | 92769 | 21: 58542608 |
| 92777 | 21: 58649278 | 92778 | 21: 58649996 | 92779 | 21: 58650822 | 92780 | 21: 58651655 | 92781 | 21: 58651789 |
| 92782 | 21: 58653170 | 92783 | 21: 58653759 | 92784 | 21: 58653796 | 92785 | 21: 58654257 | 92786 | 21: 58657343 |
| 92787 | 21: 58730952 | 92792 | 21: 58844584 | 92794 | 21: 58897764 | 92812 | 21: 59280395 | 92813 | 21: 59290376 |
| 92816 | 21: 59410654 | 92817 | 21: 59450928 | 92821 | 21: 59494769 | 92838 | 21: 59798491 | 92846 | 21: 59885566 |
| 92847 | 21: 59892560 | 92854 | 21: 59967817 | 92857 | 21: 59995456 | 92858 | 21: 60034051 | 92860 | 21: 60070408 |
| 92864 | 21: 60128571 | 92867 | 21: 60147487 | 92870 | 21: 60222745 | 92872 | 21: 60258085 | 92875 | 21: 60285746 |
| 92878 | 21: 60309341 | 92879 | 21: 60318597 | 92880 | 21: 60364407 | 92887 | 21: 60531623 | 92888 | 21: 60531761 |
| 92914 | 21: 60963159 | 92929 | 21: 61332759 | 92935 | 21: 61617246 | 92936 | 21: 61637616 | 92937 | 21: 61639799 |
| 92938 | 21: 61640498 | 92957 | 21: 62025836 | 92973 | 21: 62390233 | 93001 | 21: 63134292 | 93003 | 21: 63222235 |
| 93004 | 21: 63258242 | 93010 | 21: 63480195 | 93017 | 21: 63603801 | 93024 | 21: 63831078 | 93029 | 21: 63935578 |
| 93046 | 21: 64333571 | 93050 | 21: 64428779 | 93051 | 21: 64450577 | 93052 | 21: 64452975 | 93065 | 21: 64633547 |
| 93070 | 21: 64699689 | 93077 | 21: 64841812 | 93078 | 21: 64841843 | 93079 | 21: 64841865 | 93080 | 21: 64850271 |
| 93084 | 21: 64922535 | 93086 | 21: 64988072 | 93088 | 21: 65014550 | 93089 | 21: 65027672 | 93090 | 21: 65029870 |
| 93091 | 21: 65051618 | 93092 | 21: 65052179 | 93093 | 21: 65052717 | 93094 | 21: 65054313 | 93095 | 21: 65055762 |
| 93096 | 21: 65056739 | 93097 | 21: 65060441 | 93098 | 21: 65061116 | 93099 | 21: 65067323 | 93100 | 21: 65073410 |
| 93101 | 21: 65073892 | 93102 | 21: 65075325 | 93103 | 21: 65079623 | 93104 | 21: 65080143 | 93105 | 21: 65082481 |
| 93114 | 21: 65232598 | 93167 | 21: 66311776 | 93183 | 21: 66723461 | 93189 | 21: 66870945 | 93190 | 21: 66929334 |
| 93191 | 21: 66932880 | 93192 | 21: 66934300 | 93193 | 21: 66936241 | 93194 | 21: 66938188 | 93195 | 21: 66942278 |
| 93197 | 21: 66945357 | 93204 | 21: 67094952 | 93208 | 21: 67149363 | 93215 | 21: 67449798 | 93221 | 21: 67552612 |
| 93231 | 21: 67865058 | 93232 | 21: 67876373 | 93240 | 21: 67965438 | 93251 | 21: 68196501 | 93252 | 21: 68202138 |
| 93253 | 21: 68202185 | 93255 | 21: 68230479 | 93258 | 21: 68286931 | 93260 | 21: 68335052 | 93268 | 21: 68506930 |
| 93270 | 21: 68519125 | 93271 | 21: 68522170 | 93272 | 21: 68529965 | 93273 | 21: 68530561 | 93274 | 21: 68530572 |
| 93276 | 21: 68537086 | 93280 | 21: 68668451 | 93294 | 21: 69004265 | 93301 | 21: 69165607 | 93304 | 21: 69229046 |
| 93308 | 21: 69352283 | 93309 | 21: 69352557 | 93310 | 21: 69353990 | 93312 | 21: 69409067 | 93314 | 21: 69440442 |
| 93315 | 21: 69440464 | 93316 | 21: 69440466 | 93317 | 21: 69442039 | 93320 | 21: 69557781 | 93321 | 21: 69574694 |
| 93322 | 21: 69582716 | 93324 | 21: 69629308 | 93325 | 21: 69642755 | 93326 | 21: 69660286 | 93331 | 21: 69725960 |
| 93332 | 21: 69764707 | 93333 | 21: 69765916 | 93334 | 21: 69766081 | 93335 | 21: 69770916 | 93345 | 22: 378758 |
| 93353 | 22: 651171 | 93363 | 22: 912338 | 93366 | 22: 940171 | 93367 | 22: 940370 | 93395 | 22: 1560877 |
| 93401 | 22: 1738773 | 93402 | 22: 1738804 | 93403 | 22: 1738805 | 93404 | 22: 1746536 | 93414 | 22: 1927654 |
| 93432 | 22: 2329624 | 93449 | 22: 2751520 | 93450 | 22: 2752726 | 93456 | 22: 2877258 | 93457 | 22: 2892709 |
| 93465 | 22: 3056974 | 93471 | 22: 3197001 | 93473 | 22: 3220010 | 93475 | 22: 3269948 | 93482 | 22: 3359434 |
| 93493 | 22: 3596980 | 93499 | 22: 3798423 | 93501 | 22: 3834731 | 93504 | 22: 3914101 | 93506 | 22: 3960951 |
| 93529 | 22: 4497784 | 93540 | 22: 4729907 | 93546 | 22: 4842711 | 93551 | 22: 4922676 | 93556 | 22: 5030837 |
| 93559 | 22: 5120220 | 93561 | 22: 5160439 | 93562 | 22: 5165375 | 93564 | 22: 5179197 | 93575 | 22: 5341524 |
| 93576 | 22: 5345473 | 93577 | 22: 5391435 | 93584 | 22: 5485152 | 93595 | 22: 5647094 | 93597 | 22: 5693798 |
| 93598 | 22: 5721636 | 93600 | 22: 5764445 | 93605 | 22: 5883723 | 93612 | 22: 5950788 | 93616 | 22: 6008417 |
| 93617 | 22: 6017167 | 93618 | 22: 6052161 | 93622 | 22: 6199633 | 93627 | 22: 6252438 | 93630 | 22: 6293377 |
| 93636 | 22: 6370667 | 93643 | 22: 6488129 | 93645 | 22: 6498701 | 93657 | 22: 6784495 | 93660 | 22: 6839952 |
| 93671 | 22: 6996393 | 93685 | 22: 7428239 | 93686 | 22: 7434501 | 93697 | 22: 7651542 | 93702 | 22: 7841139 |
| 93703 | 22: 7908696 | 93723 | 22: 8411667 | 93735 | 22: 8761701 | 93772 | 22: 9753217 | 93774 | 22: 9795347 |
| 93783 | 22: 10006268 | 93784 | 22: 10006273 | 93785 | 22: 10006376 | 93787 | 22: 10035911 | 93789 | 22: 10081106 |
| 93790 | 22: 10081118 | 93791 | 22: 10081165 | 93792 | 22: 10081187 | 93793 | 22: 10081208 | 93794 | 22: 10081215 |
| 93795 | 22: 10081251 | 93796 | 22: 10081295 | 93801 | 22: 10172170 | 93804 | 22: 10236047 | 93809 | 22: 10290595 |
| 93810 | 22: 10345715 | 93812 | 22: 10401777 | 93836 | 22: 11003544 | 93841 | 22: 11122201 | 93850 | 22: 11391522 |
| 93858 | 22: 11578648 | 93860 | 22: 11718954 | 93861 | 22: 11720712 | 93865 | 22: 11758613 | 93867 | 22: 11831403 |
| 93868 | 22: 11832624 | 93869 | 22: 11889790 | 93870 | 22: 11889853 | 93871 | 22: 11890006 | 93872 | 22: 11890127 |
| 93878 | 22: 11973585 | 93879 | 22: 11977784 | 93880 | 22: 11977859 | 93881 | 22: 11978609 | 93882 | 22: 11978855 |
| 93883 | 22: 11978903 | 93886 | 22: 12019884 | 93892 | 22: 12195260 | 93897 | 22: 12303365 | 93900 | 22: 12377148 |
| 93901 | 22: 12430483 | 93902 | 22: 12436371 | 93904 | 22: 12451350 | 93907 | 22: 12503200 | 93908 | 22: 12506458 |
| 93917 | 22: 12662972 | 93921 | 22: 12724433 | 93930 | 22: 12923416 | 93936 | 22: 13091338 | 93943 | 22: 13359849 |
| 93951 | 22: 13559915 | 93952 | 22: 13582865 | 93969 | 22: 14142677 | 93970 | 22: 14145371 | 93973 | 22: 14181865 |
| 93976 | 22: 14213464 | 93978 | 22: 14312375 | 93984 | 22: 14511531 | 93985 | 22: 14512264 | 93986 | 22: 14516780 |
| 93987 | 22: 14516787 | 93988 | 22: 14518692 | 93989 | 22: 14519180 | 94006 | 22: 15072933 | 94008 | 22: 15123459 |
| 94019 | 22: 15471657 | 94022 | 22: 15504111 | 94027 | 22: 15605731 | 94051 | 22: 16322403 | 94052 | 22: 16322451 |
| 94053 | 22: 16322488 | 94054 | 22: 16353524 | 94062 | 22: 16546381 | 94063 | 22: 16548801 | 94064 | 22: 16561111 |
| 94065 | 22: 16567113 | 94066 | 22: 16567586 | 94067 | 22: 16568933 | 94073 | 22: 16666585 | 94077 | 22: 16725586 |
| 94078 | 22: 16728120 | 94080 | 22: 16741450 | 94082 | 22: 16757417 | 94083 | 22: 16773003 | 94084 | 22: 16802200 |
| 94085 | 22: 16804567 | 94100 | 22: 17418863 | 94113 | 22: 17718315 | 94116 | 22: 17791469 | 94123 | 22: 17937879 |
| 94125 | 22: 17953330 | 94130 | 22: 18035176 | 94132 | 22: 18061536 | 94151 | 22: 18541264 | 94156 | 22: 18614346 |
| 94160 | 22: 18696586 | 94164 | 22: 18832117 | 94175 | 22: 19093982 | 94177 | 22: 19212118 | 94185 | 22: 19376422 |
| 94215 | 22: 20299573 | 94221 | 22: 20368774 | 94234 | 22: 20830719 | 94259 | 22: 21264256 | 94260 | 22: 21281505 |
| 94262 | 22: 21343700 | 94270 | 22: 21553615 | 94272 | 22: 21581948 | 94275 | 22: 21638280 | 94282 | 22: 21687964 |
| 94284 | 22: 21691311 | 94285 | 22: 21691369 | 94286 | 22: 21696971 | 94287 | 22: 21697320 | 94289 | 22: 21780680 |
| 94295 | 22: 21936689 | 94298 | 22: 22018100 | 94301 | 22: 22096896 | 94302 | 22: 22130081 | 94304 | 22: 22207036 |
| 94305 | 22: 22280513 | 94306 | 22: 22280554 | 94312 | 22: 22511637 | 94321 | 22: 22847965 | 94325 | 22: 22910885 |
| 94328 | 22: 22978479 | 94390 | 22: 24919962 | 94419 | 22: 25887179 | 94426 | 22: 26004558 | 94427 | 22: 26004600 |
| 94428 | 22: 26006402 | 94429 | 22: 26028973 | 94431 | 22: 26048198 | 94432 | 22: 26048256 | 94433 | 22: 26055574 |
| 94437 | 22: 26092272 | 94438 | 22: 26092293 | 94442 | 22: 26179382 | 94443 | 22: 26199906 | 94446 | 22: 26342475 |
| 94474 | 22: 27220605 | 94480 | 22: 27400091 | 94490 | 22: 27635934 | 94507 | 22: 28135962 | 94512 | 22: 28188835 |
| 94514 | 22: 28211359 | 94518 | 22: 28297855 | 94523 | 22: 28404142 | 94524 | 22: 28404219 | 94529 | 22: 28481900 |
| 94531 | 22: 28515749 | 94533 | 22: 28587264 | 94534 | 22: 28588309 | 94539 | 22: 28733810 | 94541 | 22: 28784852 |
| 94542 | 22: 28784985 | 94543 | 22: 28792168 | 94545 | 22: 28835841 | 94546 | 22: 28835907 | 94547 | 22: 28835973 |
| 94548 | 22: 28840124 | 94549 | 22: 28840129 | 94550 | 22: 28843084 | 94551 | 22: 28844382 | 94552 | 22: 28845975 |
| 94554 | 22: 28862947 | 94555 | 22: 28938955 | 94557 | 22: 29005992 | 94566 | 22: 29299265 | 94581 | 22: 29705813 |
| 94582 | 22: 29709388 | 94583 | 22: 29712615 | 94604 | 22: 30274003 | 94607 | 22: 30417109 | 94608 | 22: 30436771 |
| 94610 | 22: 30515471 | 94624 | 22: 30808716 | 94638 | 22: 31133292 | 94639 | 22: 31135175 | 94640 | 22: 31141034 |
| 94661 | 22: 31835374 | 94662 | 22: 31861512 | 94663 | 22: 31891766 | 94666 | 22: 32022974 | 94673 | 22: 32156119 |
| 94674 | 22: 32186880 | 94675 | 22: 32233824 | 94676 | 22: 32246231 | 94679 | 22: 32399020 | 94685 | 22: 32572065 |
| 94688 | 22: 32679434 | 94698 | 22: 32982493 | 94700 | 22: 32993742 | 94701 | 22: 33017003 | 94702 | 22: 33053278 |
| 94708 | 22: 33142515 | 94728 | 22: 33935530 | 94729 | 22: 33944757 | 94733 | 22: 33983138 | 94768 | 22: 34968813 |
| 94771 | 22: 35012254 | 94774 | 22: 35039773 | 94781 | 22: 35173959 | 94791 | 22: 35329495 | 94793 | 22: 35423874 |
| 94796 | 22: 35459751 | 94797 | 22: 35460784 | 94799 | 22: 35523854 | 94810 | 22: 35736939 | 94812 | 22: 35774994 |
| 94815 | 22: 35862159 | 94816 | 22: 35890192 | 94817 | 22: 35901222 | 94826 | 22: 36031535 | 94837 | 22: 36381197 |
| 94844 | 22: 36496828 | 94846 | 22: 36551821 | 94848 | 22: 36609940 | 94856 | 22: 36967669 | 94865 | 22: 37191405 |
| 94866 | 22: 37210076 | 94872 | 22: 37384626 | 94873 | 22: 37391008 | 94876 | 22: 37473284 | 94883 | 22: 37617112 |
| 94884 | 22: 37620082 | 94885 | 22: 37622201 | 94886 | 22: 37624592 | 94887 | 22: 37627226 | 94888 | 22: 37628993 |
| 94889 | 22: 37635996 | 94890 | 22: 37636848 | 94891 | 22: 37637997 | 94892 | 22: 37640217 | 94893 | 22: 37653222 |
| 94917 | 22: 38367562 | 94919 | 22: 38384223 | 94924 | 22: 38493420 | 94928 | 22: 38548453 | 94934 | 22: 38617876 |
| 94944 | 22: 38801073 | 94955 | 22: 39102590 | 94963 | 22: 39227562 | 94964 | 22: 39234430 | 94965 | 22: 39240885 |
| 94977 | 22: 39399375 | 94978 | 22: 39399383 | 94996 | 22: 39664313 | 95001 | 22: 39736141 | 95002 | 22: 39736166 |
| 95003 | 22: 39742270 | 95005 | 22: 39764001 | 95007 | 22: 39806230 | 95022 | 22: 40159960 | 95028 | 22: 40412439 |
| 95030 | 22: 40474842 | 95031 | 22: 40497320 | 95033 | 22: 40497948 | 95036 | 22: 40553964 | 95043 | 22: 40698664 |
| 95044 | 22: 40699122 | 95046 | 22: 40709407 | 95064 | 22: 41331703 | 95077 | 22: 41673525 | 95078 | 22: 41697991 |
| 95104 | 22: 42421167 | 95108 | 22: 42552768 | 95118 | 22: 42753833 | 95120 | 22: 42860068 | 95131 | 22: 43070171 |
| 95133 | 22: 43103372 | 95135 | 22: 43152197 | 95137 | 22: 43171695 | 95139 | 22: 43185881 | 95142 | 22: 43216398 |
| 95145 | 22: 43241133 | 95148 | 22: 43280264 | 95151 | 22: 43327456 | 95152 | 22: 43328177 | 95158 | 22: 43532708 |
| 95162 | 22: 43617671 | 95170 | 22: 43831970 | 95172 | 22: 43860815 | 95190 | 22: 44240079 | 95191 | 22: 44242443 |
| 95195 | 22: 44303591 | 95212 | 22: 44704423 | 95217 | 22: 44800260 | 95225 | 22: 45101725 | 95232 | 22: 45343274 |
| 95245 | 22: 45656061 | 95247 | 22: 45691255 | 95252 | 22: 45783940 | 95255 | 22: 45826883 | 95270 | 22: 46040006 |
| 95273 | 22: 46071530 | 95291 | 22: 46619809 | 95296 | 22: 46740639 | 95298 | 22: 46748886 | 95311 | 22: 46981263 |
| 95312 | 22: 46981314 | 95315 | 22: 47033448 | 95318 | 22: 47057561 | 95331 | 22: 47235742 | 95333 | 22: 47261646 |
| 95337 | 22: 47402038 | 95342 | 22: 47467741 | 95349 | 22: 47617100 | 95352 | 22: 47677699 | 95356 | 22: 47817718 |
| 95361 | 22: 48000435 | 95364 | 22: 48019220 | 95369 | 22: 48066613 | 95370 | 22: 48091175 | 95371 | 22: 48107831 |
| 95372 | 22: 48170389 | 95373 | 22: 48171831 | 95380 | 22: 48435818 | 95381 | 22: 48449692 | 95386 | 22: 48687235 |
| 95388 | 22: 48709706 | 95391 | 22: 48761536 | 95395 | 22: 48961009 | 95396 | 22: 48979936 | 95397 | 22: 48988156 |
| 95398 | 22: 49025901 | 95399 | 22: 49040313 | 95400 | 22: 49041656 | 95405 | 22: 49152003 | 95407 | 22: 49218903 |
| 95411 | 22: 49396397 | 95412 | 22: 49411257 | 95413 | 22: 49416701 | 95417 | 22: 49475649 | 95422 | 22: 49751795 |
| 95427 | 22: 49845416 | 95428 | 22: 49855390 | 95429 | 22: 49862171 | 95430 | 22: 49874759 | 95437 | 22: 50031711 |
| 95443 | 22: 50110984 | 95444 | 22: 50127865 | 95446 | 22: 50174136 | 95448 | 22: 50267951 | 95453 | 22: 50398849 |
| 95463 | 22: 50615575 | 95464 | 22: 50626022 | 95467 | 22: 50669330 | 95468 | 22: 50669331 | 95471 | 22: 50732557 |
| 95472 | 22: 50752636 | 95475 | 22: 50849444 | 95488 | 22: 51284290 | 95490 | 22: 51317516 | 95495 | 22: 51400864 |
| 95502 | 22: 51573666 | 95509 | 22: 51800322 | 95517 | 22: 51985899 | 95518 | 22: 51994258 | 95521 | 22: 52090420 |
| 95522 | 22: 52090421 | 95523 | 22: 52090571 | 95526 | 22: 52113515 | 95528 | 22: 52142954 | 95535 | 22: 52398536 |
| 95536 | 22: 52409661 | 95545 | 22: 52642109 | 95547 | 22: 52689484 | 95555 | 22: 52876456 | 95559 | 22: 52889478 |
| 95562 | 22: 52940887 | 95567 | 22: 52998347 | 95568 | 22: 52998797 | 95569 | 22: 52998800 | 95570 | 22: 52999211 |
| 95571 | 22: 52999397 | 95573 | 22: 53017868 | 95586 | 22: 53333639 | 95595 | 22: 53587524 | 95596 | 22: 53630193 |
| 95597 | 22: 53632109 | 95599 | 22: 53667747 | 95601 | 22: 53701852 | 95603 | 22: 53750298 | 95604 | 22: 53771417 |
| 95608 | 22: 53814947 | 95609 | 22: 53815430 | 95610 | 22: 53823185 | 95617 | 22: 53952061 | 95625 | 22: 54050342 |
| 95626 | 22: 54084951 | 95634 | 22: 54271229 | 95636 | 22: 54325220 | 95637 | 22: 54325294 | 95638 | 22: 54326413 |
| 95639 | 22: 54326417 | 95640 | 22: 54326525 | 95652 | 22: 54582223 | 95654 | 22: 54586616 | 95655 | 22: 54590257 |
| 95656 | 22: 54591674 | 95657 | 22: 54601062 | 95659 | 22: 54619999 | 95660 | 22: 54656963 | 95661 | 22: 54657085 |
| 95681 | 22: 55222425 | 95682 | 22: 55249217 | 95684 | 22: 55290303 | 95685 | 22: 55290330 | 95687 | 22: 55290410 |
| 95688 | 22: 55290479 | 95689 | 22: 55290914 | 95690 | 22: 55291002 | 95691 | 22: 55291168 | 95692 | 22: 55291236 |
| 95693 | 22: 55291381 | 95694 | 22: 55291382 | 95697 | 22: 55338384 | 95699 | 22: 55379431 | 95700 | 22: 55413027 |
| 95705 | 22: 55446332 | 95706 | 22: 55455982 | 95709 | 22: 55483225 | 95713 | 22: 55579849 | 95725 | 22: 55804900 |
| 95728 | 22: 55847268 | 95733 | 22: 55887174 | 95734 | 22: 55887346 | 95735 | 22: 55887394 | 95736 | 22: 55887397 |
| 95737 | 22: 55887398 | 95738 | 22: 55887409 | 95739 | 22: 55887425 | 95740 | 22: 55887575 | 95741 | 22: 55888374 |
| 95746 | 22: 55952909 | 95759 | 22: 56301696 | 95761 | 22: 56325207 | 95762 | 22: 56325210 | 95763 | 22: 56325225 |
| 95764 | 22: 56325235 | 95765 | 22: 56325272 | 95766 | 22: 56325600 | 95767 | 22: 56325606 | 95769 | 22: 56325745 |
| 95770 | 22: 56325754 | 95771 | 22: 56326527 | 95772 | 22: 56329687 | 95773 | 22: 56329721 | 95774 | 22: 56332166 |
| 95775 | 22: 56340185 | 95777 | 22: 56377918 | 95778 | 22: 56388281 | 95780 | 22: 56396437 | 95791 | 22: 56484402 |
| 95792 | 22: 56513273 | 95797 | 22: 56546272 | 95800 | 22: 56616418 | 95801 | 22: 56632616 | 95822 | 22: 57017818 |
| 95823 | 22: 57017940 | 95825 | 22: 57074332 | 95827 | 22: 57132556 | 95830 | 22: 57180726 | 95831 | 22: 57198542 |
| 95832 | 22: 57228439 | 95833 | 22: 57257734 | 95834 | 22: 57268915 | 95840 | 22: 57427960 | 95841 | 22: 57434193 |
| 95843 | 22: 57453058 | 95846 | 22: 57478438 | 95847 | 22: 57511375 | 95851 | 22: 57532676 | 95855 | 22: 57579073 |
| 95857 | 22: 57629519 | 95858 | 22: 57638240 | 95869 | 22: 57759457 | 95870 | 22: 57764469 | 95871 | 22: 57766664 |
| 95872 | 22: 57792445 | 95890 | 22: 58069483 | 95891 | 22: 58075933 | 95892 | 22: 58086195 | 95905 | 22: 58427446 |
| 95907 | 22: 58446882 | 95911 | 22: 58506677 | 95925 | 22: 58804900 | 95938 | 22: 59105276 | 95939 | 22: 59111787 |
| 95945 | 22: 59165452 | 95947 | 22: 59174986 | 95950 | 22: 59237930 | 95961 | 22: 59518872 | 95966 | 22: 59625926 |
| 95978 | 22: 59729840 | 95982 | 22: 59783092 | 95983 | 22: 59831717 | 95989 | 22: 59928044 | 96005 | 22: 60263301 |
| 96007 | 22: 60314302 | 96008 | 22: 60344480 | 96009 | 22: 60369311 | 96013 | 22: 60433161 | 96014 | 22: 60450289 |
| 96015 | 22: 60450822 | 96016 | 22: 60450826 | 96021 | 22: 60583428 | 96024 | 22: 60664148 | 96028 | 23: 98715 |
| 96048 | 23: 908722 | 96059 | 23: 1233960 | 96067 | 23: 1413088 | 96081 | 23: 1806494 | 96089 | 23: 2027290 |
| 96091 | 23: 2085668 | 96102 | 23: 2295458 | 96103 | 23: 2327306 | 96109 | 23: 2490639 | 96115 | 23: 2633185 |
| 96119 | 23: 2653923 | 96121 | 23: 2672734 | 96122 | 23: 2681218 | 96131 | 23: 2932961 | 96132 | 23: 2932963 |
| 96133 | 23: 2933026 | 96134 | 23: 2933089 | 96135 | 23: 2933119 | 96136 | 23: 2943350 | 96137 | 23: 2944259 |
| 96138 | 23: 2945377 | 96147 | 23: 3182108 | 96150 | 23: 3278552 | 96152 | 23: 3363897 | 96154 | 23: 3397926 |
| 96155 | 23: 3401808 | 96156 | 23: 3422753 | 96157 | 23: 3441692 | 96158 | 23: 3451376 | 96167 | 23: 3623220 |
| 96169 | 23: 3649638 | 96172 | 23: 3661124 | 96173 | 23: 3691486 | 96174 | 23: 3694848 | 96187 | 23: 3953291 |
| 96195 | 23: 4055037 | 96207 | 23: 4270316 | 96211 | 23: 4397147 | 96257 | 23: 5221777 | 96270 | 23: 5551234 |
| 96273 | 23: 5596536 | 96274 | 23: 5598882 | 96275 | 23: 5598939 | 96276 | 23: 5598954 | 96277 | 23: 5598967 |
| 96291 | 23: 6007499 | 96302 | 23: 6313109 | 96315 | 23: 6596002 | 96316 | 23: 6596059 | 96319 | 23: 6615760 |
| 96320 | 23: 6619709 | 96321 | 23: 6619777 | 96334 | 23: 6834687 | 96343 | 23: 7086052 | 96348 | 23: 7176749 |
| 96349 | 23: 7185030 | 96351 | 23: 7220926 | 96354 | 23: 7287024 | 96362 | 23: 7470856 | 96366 | 23: 7588787 |
| 96367 | 23: 7590703 | 96368 | 23: 7604788 | 96369 | 23: 7627114 | 96370 | 23: 7627927 | 96385 | 23: 8019039 |
| 96386 | 23: 8021311 | 96388 | 23: 8043634 | 96394 | 23: 8257417 | 96395 | 23: 8265499 | 96402 | 23: 8382452 |
| 96403 | 23: 8386569 | 96405 | 23: 8392718 | 96406 | 23: 8394550 | 96407 | 23: 8403739 | 96409 | 23: 8430336 |
| 96410 | 23: 8437470 | 96411 | 23: 8444830 | 96412 | 23: 8458254 | 96423 | 23: 8693541 | 96428 | 23: 8861332 |
| 96429 | 23: 8861347 | 96435 | 23: 8977884 | 96438 | 23: 9024159 | 96439 | 23: 9024206 | 96440 | 23: 9024246 |
| 96447 | 23: 9163290 | 96448 | 23: 9164368 | 96452 | 23: 9341201 | 96457 | 23: 9496576 | 96460 | 23: 9564042 |
| 96461 | 23: 9565542 | 96462 | 23: 9583880 | 96464 | 23: 9685892 | 96466 | 23: 9745811 | 96467 | 23: 9767204 |
| 96476 | 23: 10029783 | 96483 | 23: 10140851 | 96485 | 23: 10256230 | 96486 | 23: 10257763 | 96487 | 23: 10257827 |
| 96488 | 23: 10258971 | 96489 | 23: 10259318 | 96490 | 23: 10259504 | 96492 | 23: 10261010 | 96493 | 23: 10262012 |
| 96494 | 23: 10262085 | 96495 | 23: 10262281 | 96496 | 23: 10262341 | 96497 | 23: 10262386 | 96498 | 23: 10262523 |
| 96501 | 23: 10328877 | 96506 | 23: 10459370 | 96514 | 23: 10615240 | 96515 | 23: 10638380 | 96519 | 23: 10710493 |
| 96523 | 23: 10803832 | 96524 | 23: 10870495 | 96527 | 23: 10883412 | 96528 | 23: 10883649 | 96529 | 23: 10890338 |
| 96530 | 23: 10913209 | 96532 | 23: 10921995 | 96533 | 23: 10922014 | 96538 | 23: 11106125 | 96554 | 23: 11574894 |
| 96555 | 23: 11592749 | 96556 | 23: 11599745 | 96564 | 23: 11737588 | 96566 | 23: 11785803 | 96573 | 23: 11925495 |
| 96575 | 23: 11950761 | 96576 | 23: 11956619 | 96586 | 23: 12073751 | 96591 | 23: 12208427 | 96594 | 23: 12235079 |
| 96603 | 23: 12561054 | 96604 | 23: 12561114 | 96605 | 23: 12561116 | 96606 | 23: 12561252 | 96607 | 23: 12561302 |
| 96608 | 23: 12564591 | 96609 | 23: 12566537 | 96625 | 23: 12975952 | 96626 | 23: 13002407 | 96631 | 23: 13062813 |
| 96632 | 23: 13062870 | 96633 | 23: 13063271 | 96634 | 23: 13065594 | 96635 | 23: 13065660 | 96636 | 23: 13066259 |
| 96637 | 23: 13073019 | 96638 | 23: 13083039 | 96639 | 23: 13096550 | 96640 | 23: 13119734 | 96645 | 23: 13155594 |
| 96650 | 23: 13360736 | 96668 | 23: 13797670 | 96669 | 23: 13803074 | 96673 | 23: 13844075 | 96682 | 23: 13890619 |
| 96683 | 23: 13913723 | 96684 | 23: 13917180 | 96711 | 23: 14503938 | 96713 | 23: 14550205 | 96726 | 23: 14801418 |
| 96736 | 23: 14938004 | 96740 | 23: 14977766 | 96744 | 23: 15085370 | 96746 | 23: 15116016 | 96747 | 23: 15116055 |
| 96748 | 23: 15118596 | 96749 | 23: 15119248 | 96750 | 23: 15119542 | 96751 | 23: 15119571 | 96752 | 23: 15119572 |
| 96753 | 23: 15119619 | 96754 | 23: 15135050 | 96761 | 23: 15271919 | 96763 | 23: 15283396 | 96766 | 23: 15345255 |
| 96773 | 23: 15533652 | 96778 | 23: 15581761 | 96795 | 23: 15759615 | 96801 | 23: 15883997 | 96803 | 23: 15932643 |
| 96813 | 23: 16272106 | 96820 | 23: 16456191 | 96822 | 23: 16466008 | 96823 | 23: 16479564 | 96829 | 23: 16661447 |
| 96831 | 23: 16686014 | 96832 | 23: 16714066 | 96835 | 23: 16792993 | 96836 | 23: 16801536 | 96843 | 23: 17062554 |
| 96859 | 23: 17556957 | 96868 | 23: 17708671 | 96869 | 23: 17718607 | 96870 | 23: 17737639 | 96890 | 23: 18161337 |
| 96891 | 23: 18200714 | 96894 | 23: 18216510 | 96935 | 23: 19331450 | 96936 | 23: 19366522 | 96939 | 23: 19415329 |
| 96966 | 23: 19751742 | 96970 | 23: 19767163 | 96974 | 23: 19826206 | 96976 | 23: 19925315 | 96982 | 23: 19970969 |
| 96985 | 23: 20042631 | 96986 | 23: 20052143 | 96989 | 23: 20102219 | 96991 | 23: 20121033 | 96997 | 23: 20203703 |
| 96998 | 23: 20223241 | 97007 | 23: 20409619 | 97008 | 23: 20409671 | 97009 | 23: 20409761 | 97024 | 23: 20698756 |
| 97025 | 23: 20786968 | 97028 | 23: 20835335 | 97029 | 23: 20835446 | 97045 | 23: 21265221 | 97050 | 23: 21344891 |
| 97056 | 23: 21453135 | 97057 | 23: 21453252 | 97066 | 23: 21870117 | 97085 | 23: 22255326 | 97088 | 23: 22364773 |
| 97113 | 23: 23196063 | 97126 | 23: 23576448 | 97135 | 23: 23755239 | 97136 | 23: 23762207 | 97138 | 23: 23782323 |
| 97148 | 23: 23940335 | 97149 | 23: 23968151 | 97156 | 23: 24092418 | 97164 | 23: 24300414 | 97165 | 23: 24303099 |
| 97167 | 23: 24343369 | 97168 | 23: 24345237 | 97169 | 23: 24352270 | 97173 | 23: 24416257 | 97174 | 23: 24423273 |
| 97179 | 23: 24498401 | 97180 | 23: 24505272 | 97186 | 23: 24683101 | 97187 | 23: 24685752 | 97188 | 23: 24686739 |
| 97189 | 23: 24689461 | 97190 | 23: 24692653 | 97191 | 23: 24699153 | 97192 | 23: 24700095 | 97193 | 23: 24701041 |
| 97194 | 23: 24702797 | 97195 | 23: 24703647 | 97196 | 23: 24704414 | 97197 | 23: 24705785 | 97198 | 23: 24709728 |
| 97200 | 23: 24717517 | 97201 | 23: 24718990 | 97202 | 23: 24723139 | 97203 | 23: 24727842 | 97204 | 23: 24729644 |
| 97205 | 23: 24730615 | 97206 | 23: 24732566 | 97207 | 23: 24733786 | 97208 | 23: 24736986 | 97209 | 23: 24739399 |
| 97210 | 23: 24744157 | 97213 | 23: 24790792 | 97214 | 23: 24794179 | 97216 | 23: 24799157 | 97217 | 23: 24800192 |
| 97218 | 23: 24823698 | 97219 | 23: 24823737 | 97224 | 23: 24880628 | 97226 | 23: 24916092 | 97228 | 23: 24934787 |
| 97229 | 23: 24936283 | 97230 | 23: 24939717 | 97232 | 23: 24975703 | 97240 | 23: 25055772 | 97243 | 23: 25088466 |
| 97244 | 23: 25089919 | 97245 | 23: 25102180 | 97247 | 23: 25129357 | 97248 | 23: 25130486 | 97249 | 23: 25143923 |
| 97250 | 23: 25145004 | 97251 | 23: 25146146 | 97252 | 23: 25156088 | 97253 | 23: 25164433 | 97254 | 23: 25166831 |
| 97257 | 23: 25198505 | 97258 | 23: 25199834 | 97259 | 23: 25200752 | 97260 | 23: 25201563 | 97264 | 23: 25222382 |
| 97265 | 23: 25235072 | 97267 | 23: 25235958 | 97269 | 23: 25250609 | 97271 | 23: 25276948 | 97272 | 23: 25283721 |
| 97273 | 23: 25287389 | 97275 | 23: 25323788 | 97279 | 23: 25354558 | 97280 | 23: 25354656 | 97282 | 23: 25372486 |
| 97286 | 23: 25406424 | 97287 | 23: 25406813 | 97289 | 23: 25433764 | 97292 | 23: 25467868 | 97296 | 23: 25560755 |
| 97298 | 23: 25617278 | 97299 | 23: 25674209 | 97300 | 23: 25680001 | 97301 | 23: 25689132 | 97302 | 23: 25690596 |
| 97303 | 23: 25692139 | 97304 | 23: 25693451 | 97306 | 23: 25715869 | 97308 | 23: 25730240 | 97309 | 23: 25730292 |
| 97310 | 23: 25730339 | 97311 | 23: 25750106 | 97312 | 23: 25755389 | 97313 | 23: 25758772 | 97314 | 23: 25759863 |
| 97315 | 23: 25764182 | 97316 | 23: 25767576 | 97317 | 23: 25780173 | 97318 | 23: 25781822 | 97319 | 23: 25786244 |
| 97320 | 23: 25788400 | 97322 | 23: 25818270 | 97323 | 23: 25819563 | 97324 | 23: 25819612 | 97325 | 23: 25819634 |
| 97327 | 23: 25825333 | 97328 | 23: 25827861 | 97330 | 23: 25834509 | 97331 | 23: 25840572 | 97332 | 23: 25842401 |
| 97335 | 23: 25902950 | 97352 | 23: 26938031 | 97353 | 23: 26943744 | 97354 | 23: 26947613 | 97360 | 23: 27113071 |
| 97361 | 23: 27113077 | 97362 | 23: 27113082 | 97363 | 23: 27113092 | 97364 | 23: 27113093 | 97366 | 23: 27139603 |
| 97367 | 23: 27139619 | 97368 | 23: 27139643 | 97369 | 23: 27141360 | 97370 | 23: 27144341 | 97372 | 23: 27175642 |
| 97373 | 23: 27176260 | 97379 | 23: 27233819 | 97381 | 23: 27306574 | 97382 | 23: 27317678 | 97383 | 23: 27325182 |
| 97384 | 23: 27348931 | 97385 | 23: 27355151 | 97386 | 23: 27356345 | 97387 | 23: 27360333 | 97388 | 23: 27361033 |
| 97389 | 23: 27361247 | 97390 | 23: 27361255 | 97392 | 23: 27385789 | 97393 | 23: 27397427 | 97394 | 23: 27397904 |
| 97395 | 23: 27398878 | 97402 | 23: 27492296 | 97403 | 23: 27493205 | 97404 | 23: 27494111 | 97406 | 23: 27497189 |
| 97407 | 23: 27497222 | 97408 | 23: 27497848 | 97409 | 23: 27497875 | 97410 | 23: 27497880 | 97411 | 23: 27499647 |
| 97412 | 23: 27514878 | 97413 | 23: 27516482 | 97414 | 23: 27522712 | 97415 | 23: 27528967 | 97417 | 23: 27538128 |
| 97419 | 23: 27545924 | 97421 | 23: 27601917 | 97425 | 23: 27635451 | 97426 | 23: 27643343 | 97428 | 23: 27657685 |
| 97431 | 23: 27701786 | 97432 | 23: 27727500 | 97435 | 23: 27774805 | 97436 | 23: 27801348 | 97437 | 23: 27815800 |
| 97438 | 23: 27824468 | 97440 | 23: 27883512 | 97441 | 23: 27910429 | 97442 | 23: 27976925 | 97443 | 23: 27978843 |
| 97445 | 23: 27985061 | 97446 | 23: 27988153 | 97447 | 23: 27988939 | 97449 | 23: 27989644 | 97452 | 23: 28001628 |
| 97453 | 23: 28002243 | 97454 | 23: 28003650 | 97455 | 23: 28006280 | 97456 | 23: 28016941 | 97457 | 23: 28023809 |
| 97458 | 23: 28024215 | 97459 | 23: 28024237 | 97460 | 23: 28029876 | 97461 | 23: 28030196 | 97462 | 23: 28040546 |
| 97463 | 23: 28054764 | 97464 | 23: 28055141 | 97465 | 23: 28055144 | 97466 | 23: 28055147 | 97467 | 23: 28055154 |
| 97468 | 23: 28055203 | 97469 | 23: 28055205 | 97470 | 23: 28056174 | 97471 | 23: 28056245 | 97472 | 23: 28056261 |
| 97473 | 23: 28056279 | 97474 | 23: 28058594 | 97475 | 23: 28084930 | 97476 | 23: 28084959 | 97480 | 23: 28142109 |
| 97481 | 23: 28142880 | 97482 | 23: 28143776 | 97483 | 23: 28147241 | 97485 | 23: 28154077 | 97486 | 23: 28154081 |
| 97487 | 23: 28154567 | 97488 | 23: 28158031 | 97489 | 23: 28162469 | 97490 | 23: 28169587 | 97492 | 23: 28176948 |
| 97493 | 23: 28177862 | 97494 | 23: 28178986 | 97495 | 23: 28180501 | 97496 | 23: 28189617 | 97497 | 23: 28214057 |
| 97499 | 23: 28218286 | 97500 | 23: 28226442 | 97502 | 23: 28247153 | 97504 | 23: 28252270 | 97505 | 23: 28260136 |
| 97506 | 23: 28261564 | 97507 | 23: 28266105 | 97511 | 23: 28289161 | 97512 | 23: 28311837 | 97513 | 23: 28314357 |
| 97514 | 23: 28315369 | 97517 | 23: 28344384 | 97518 | 23: 28345650 | 97520 | 23: 28370592 | 97522 | 23: 28424137 |
| 97523 | 23: 28428619 | 97525 | 23: 28451722 | 97529 | 23: 28527686 | 97530 | 23: 28605105 | 97532 | 23: 28614214 |
| 97533 | 23: 28633036 | 97534 | 23: 28644648 | 97535 | 23: 28667350 | 97536 | 23: 28759020 | 97537 | 23: 28768059 |
| 97538 | 23: 28770375 | 97539 | 23: 28770402 | 97540 | 23: 28770465 | 97541 | 23: 28770499 | 97542 | 23: 28770500 |
| 97543 | 23: 28770527 | 97545 | 23: 28779774 | 97547 | 23: 28784189 | 97549 | 23: 28791635 | 97551 | 23: 28804972 |
| 97552 | 23: 28806779 | 97553 | 23: 28808892 | 97554 | 23: 28811155 | 97555 | 23: 28811333 | 97556 | 23: 28817232 |
| 97557 | 23: 28819161 | 97558 | 23: 28819173 | 97560 | 23: 28820599 | 97561 | 23: 28821000 | 97563 | 23: 28829534 |
| 97564 | 23: 28836068 | 97565 | 23: 28840263 | 97567 | 23: 28855995 | 97568 | 23: 28868586 | 97569 | 23: 28870611 |
| 97570 | 23: 28873879 | 97571 | 23: 28878589 | 97572 | 23: 28881735 | 97574 | 23: 28920628 | 97575 | 23: 28935375 |
| 97576 | 23: 28938250 | 97580 | 23: 28964584 | 97581 | 23: 28967619 | 97582 | 23: 28971358 | 97583 | 23: 28980855 |
| 97584 | 23: 29001677 | 97585 | 23: 29002896 | 97586 | 23: 29003973 | 97587 | 23: 29004861 | 97589 | 23: 29028277 |
| 97591 | 23: 29088900 | 97593 | 23: 29127784 | 97595 | 23: 29155100 | 97598 | 23: 29200199 | 97599 | 23: 29231778 |
| 97604 | 23: 29351343 | 97605 | 23: 29351673 | 97606 | 23: 29387809 | 97608 | 23: 29405131 | 97610 | 23: 29505622 |
| 97618 | 23: 29739156 | 97619 | 23: 29739161 | 97629 | 23: 30015425 | 97633 | 23: 30085624 | 97641 | 23: 30343016 |
| 97643 | 23: 30357104 | 97644 | 23: 30377231 | 97645 | 23: 30377501 | 97646 | 23: 30391263 | 97647 | 23: 30408989 |
| 97648 | 23: 30411874 | 97649 | 23: 30417585 | 97650 | 23: 30422451 | 97651 | 23: 30442710 | 97652 | 23: 30443193 |
| 97653 | 23: 30447941 | 97654 | 23: 30464791 | 97656 | 23: 30478896 | 97660 | 23: 30538186 | 97662 | 23: 30568716 |
| 97669 | 23: 30789137 | 97670 | 23: 30789578 | 97671 | 23: 30789711 | 97685 | 23: 31163020 | 97687 | 23: 31193034 |
| 97688 | 23: 31201200 | 97689 | 23: 31207608 | 97690 | 23: 31237063 | 97691 | 23: 31239018 | 97696 | 23: 31408344 |
| 97697 | 23: 31474420 | 97698 | 23: 31478356 | 97699 | 23: 31478871 | 97706 | 23: 31666638 | 97708 | 23: 31712475 |
| 97709 | 23: 31713030 | 97710 | 23: 31713883 | 97712 | 23: 31795823 | 97716 | 23: 31839586 | 97717 | 23: 31847799 |
| 97718 | 23: 31860518 | 97719 | 23: 31864204 | 97720 | 23: 31883086 | 97721 | 23: 31885522 | 97723 | 23: 31892464 |
| 97724 | 23: 31898107 | 97731 | 23: 32103028 | 97735 | 23: 32155949 | 97748 | 23: 32348823 | 97754 | 23: 32429180 |
| 97755 | 23: 32429299 | 97756 | 23: 32429437 | 97760 | 23: 32498379 | 97763 | 23: 32587067 | 97769 | 23: 32714057 |
| 97776 | 23: 32953339 | 97777 | 23: 32953359 | 97778 | 23: 32954768 | 97779 | 23: 32963026 | 97781 | 23: 32991047 |
| 97782 | 23: 33010094 | 97783 | 23: 33010248 | 97784 | 23: 33017162 | 97793 | 23: 33209372 | 97796 | 23: 33232305 |
| 97809 | 23: 33564304 | 97823 | 23: 33923637 | 97829 | 23: 34147241 | 97832 | 23: 34255293 | 97846 | 23: 34559019 |
| 97847 | 23: 34568421 | 97872 | 23: 35579146 | 97874 | 23: 35643122 | 97882 | 23: 35853906 | 97884 | 23: 35933978 |
| 97897 | 23: 36340613 | 97903 | 23: 36491282 | 97904 | 23: 36522078 | 97911 | 23: 36715268 | 97922 | 23: 37003285 |
| 97923 | 23: 37008791 | 97926 | 23: 37147881 | 97929 | 23: 37226966 | 97931 | 23: 37255744 | 97933 | 23: 37290169 |
| 97934 | 23: 37311602 | 97938 | 23: 37438508 | 97939 | 23: 37509546 | 97948 | 23: 37644241 | 97952 | 23: 37725066 |
| 97954 | 23: 37743052 | 97964 | 23: 38103066 | 97969 | 23: 38331199 | 97970 | 23: 38380507 | 97973 | 23: 38417336 |
| 97995 | 23: 38995562 | 98005 | 23: 39248806 | 98007 | 23: 39300011 | 98009 | 23: 39319903 | 98029 | 23: 39426076 |
| 98034 | 23: 39454222 | 98046 | 23: 39542549 | 98058 | 23: 39582084 | 98065 | 23: 39599289 | 98072 | 23: 39623727 |
| 98074 | 23: 39636483 | 98076 | 23: 39640935 | 98083 | 23: 39789176 | 98085 | 23: 39815156 | 98090 | 23: 39928196 |
| 98093 | 23: 39972622 | 98097 | 23: 40044291 | 98098 | 23: 40064417 | 98106 | 23: 40177352 | 98107 | 23: 40190398 |
| 98109 | 23: 40223350 | 98111 | 23: 40255790 | 98112 | 23: 40270530 | 98116 | 23: 40376606 | 98119 | 23: 40424669 |
| 98121 | 23: 40430850 | 98123 | 23: 40480107 | 98124 | 23: 40500673 | 98125 | 23: 40564407 | 98131 | 23: 40662422 |
| 98133 | 23: 40688993 | 98136 | 23: 40758273 | 98139 | 23: 40791971 | 98156 | 23: 41037376 | 98161 | 23: 41085635 |
| 98163 | 23: 41110764 | 98177 | 23: 41370057 | 98178 | 23: 41371667 | 98179 | 23: 41393226 | 98182 | 23: 41488055 |
| 98198 | 23: 41873620 | 98228 | 23: 42474522 | 98251 | 23: 43050343 | 98257 | 23: 43142746 | 98266 | 23: 43402438 |
| 98282 | 23: 43699449 | 98288 | 23: 43890903 | 98290 | 23: 43922804 | 98325 | 23: 44517863 | 98326 | 23: 44517941 |
| 98332 | 23: 44610396 | 98341 | 23: 44856499 | 98343 | 23: 44879028 | 98344 | 23: 44896794 | 98345 | 23: 44914975 |
| 98346 | 23: 44944349 | 98354 | 23: 45035699 | 98357 | 23: 45057099 | 98358 | 23: 45063024 | 98378 | 23: 45357440 |
| 98380 | 23: 45397415 | 98381 | 23: 45398681 | 98383 | 23: 45430124 | 98384 | 23: 45432927 | 98390 | 23: 45560731 |
| 98404 | 23: 46021030 | 98405 | 23: 46022790 | 98406 | 23: 46022913 | 98407 | 23: 46023921 | 98408 | 23: 46024140 |
| 98419 | 23: 46342707 | 98424 | 23: 46476087 | 98434 | 23: 46728089 | 98439 | 23: 46821712 | 98440 | 23: 46867745 |
| 98445 | 23: 46932972 | 98447 | 23: 46982531 | 98448 | 23: 46982538 | 98449 | 23: 46982544 | 98450 | 23: 46982553 |
| 98451 | 23: 46982569 | 98452 | 23: 46982672 | 98453 | 23: 46982688 | 98469 | 23: 47346112 | 98470 | 23: 47346172 |
| 98472 | 23: 47377820 | 98476 | 23: 47501209 | 98479 | 23: 47512112 | 98480 | 23: 47535388 | 98493 | 23: 47816262 |
| 98494 | 23: 47825640 | 98495 | 23: 47825678 | 98496 | 23: 47826029 | 98497 | 23: 47826560 | 98498 | 23: 47827373 |
| 98500 | 23: 47839983 | 98501 | 23: 47840868 | 98503 | 23: 47845586 | 98504 | 23: 47847036 | 98523 | 23: 48330566 |
| 98529 | 23: 48410101 | 98533 | 23: 48581773 | 98538 | 23: 48634859 | 98545 | 23: 48734960 | 98547 | 23: 48752878 |
| 98555 | 23: 48901109 | 98567 | 23: 49079717 | 98569 | 23: 49107918 | 98574 | 23: 49198514 | 98576 | 23: 49210048 |
| 98584 | 23: 49312924 | 98585 | 23: 49312984 | 98586 | 23: 49312985 | 98587 | 23: 49312986 | 98592 | 23: 49424994 |
| 98593 | 23: 49432035 | 98594 | 23: 49442339 | 98602 | 23: 49590910 | 98622 | 23: 49941067 | 98624 | 23: 49942903 |
| 98625 | 23: 49942904 | 98632 | 23: 50043273 | 98635 | 23: 50078664 | 98636 | 23: 50114133 | 98638 | 23: 50169252 |
| 98642 | 23: 50258011 | 98649 | 23: 50375296 | 98664 | 23: 50638453 | 98671 | 23: 50875493 | 98678 | 23: 50932969 |
| 98680 | 23: 50964263 | 98686 | 23: 51018373 | 98689 | 23: 51079827 | 98691 | 23: 51116781 | 98701 | 23: 51298091 |
| 98711 | 23: 51501609 | 98718 | 23: 51674082 | 98722 | 23: 51744554 | 98728 | 23: 51847760 | 98737 | 23: 52008498 |
| 98741 | 23: 52053452 | 98743 | 23: 52076716 | 98781 | 24: 469885 | 98798 | 24: 880857 | 98799 | 24: 888643 |
| 98802 | 24: 930959 | 98805 | 24: 980184 | 98808 | 24: 1046300 | 98818 | 24: 1170762 | 98819 | 24: 1171661 |
| 98834 | 24: 1570237 | 98851 | 24: 1855510 | 98862 | 24: 2006568 | 98871 | 24: 2265624 | 98872 | 24: 2276656 |
| 98900 | 24: 2945776 | 98902 | 24: 2978064 | 98918 | 24: 3289918 | 98930 | 24: 3538543 | 98933 | 24: 3696514 |
| 98940 | 24: 3908092 | 98942 | 24: 3966055 | 98943 | 24: 3966359 | 98947 | 24: 4007654 | 98948 | 24: 4007678 |
| 98949 | 24: 4012661 | 98950 | 24: 4013403 | 98951 | 24: 4021260 | 98956 | 24: 4164775 | 98957 | 24: 4164916 |
| 98958 | 24: 4164934 | 98959 | 24: 4165918 | 98960 | 24: 4168088 | 98965 | 24: 4188159 | 98970 | 24: 4291963 |
| 98974 | 24: 4399899 | 98980 | 24: 4581542 | 98982 | 24: 4612976 | 98989 | 24: 4963101 | 98995 | 24: 5087606 |
| 99004 | 24: 5305296 | 99005 | 24: 5333407 | 99006 | 24: 5337650 | 99018 | 24: 5642653 | 99026 | 24: 5818978 |
| 99038 | 24: 6264925 | 99076 | 24: 7162546 | 99078 | 24: 7286305 | 99086 | 24: 7467215 | 99111 | 24: 8069004 |
| 99112 | 24: 8108977 | 99113 | 24: 8109036 | 99116 | 24: 8121802 | 99118 | 24: 8175362 | 99119 | 24: 8182146 |
| 99146 | 24: 9005035 | 99151 | 24: 9085950 | 99190 | 24: 9913417 | 99192 | 24: 9977557 | 99276 | 24: 10203460 |
| 99283 | 24: 10336705 | 99286 | 24: 10405660 | 99292 | 24: 10590068 | 99293 | 24: 10590091 | 99311 | 24: 11064065 |
| 99321 | 24: 11243732 | 99322 | 24: 11285722 | 99329 | 24: 11451899 | 99339 | 24: 11738379 | 99359 | 24: 12247620 |
| 99364 | 24: 12466661 | 99365 | 24: 12471212 | 99371 | 24: 12650426 | 99372 | 24: 12652362 | 99373 | 24: 12660326 |
| 99396 | 24: 13228420 | 99405 | 24: 13465765 | 99418 | 24: 13789354 | 99419 | 24: 13790831 | 99420 | 24: 13791730 |
| 99427 | 24: 14119618 | 99459 | 24: 14988931 | 99484 | 24: 15813747 | 99490 | 24: 16186692 | 99501 | 24: 16499172 |
| 99503 | 24: 16506987 | 99509 | 24: 16666243 | 99528 | 24: 17162739 | 99530 | 24: 17208682 | 99543 | 24: 17625014 |
| 99565 | 24: 18353069 | 99574 | 24: 18678355 | 99598 | 24: 19285724 | 99603 | 24: 19437043 | 99607 | 24: 19606961 |
| 99610 | 24: 19713861 | 99615 | 24: 19770957 | 99628 | 24: 20349623 | 99629 | 24: 20358408 | 99632 | 24: 20393549 |
| 99638 | 24: 20499578 | 99639 | 24: 20510429 | 99640 | 24: 20510450 | 99641 | 24: 20510487 | 99642 | 24: 20510488 |
| 99643 | 24: 20510505 | 99644 | 24: 20510579 | 99645 | 24: 20510583 | 99646 | 24: 20510587 | 99647 | 24: 20510597 |
| 99648 | 24: 20510600 | 99649 | 24: 20513392 | 99650 | 24: 20513424 | 99651 | 24: 20513517 | 99652 | 24: 20521127 |
| 99653 | 24: 20532438 | 99654 | 24: 20532441 | 99655 | 24: 20532446 | 99656 | 24: 20532502 | 99657 | 24: 20572686 |
| 99669 | 24: 20847626 | 99671 | 24: 20874971 | 99672 | 24: 20920443 | 99673 | 24: 20920491 | 99674 | 24: 20920727 |
| 99675 | 24: 20922777 | 99676 | 24: 20926914 | 99677 | 24: 20927000 | 99678 | 24: 20927024 | 99679 | 24: 20927059 |
| 99680 | 24: 20929398 | 99681 | 24: 20929449 | 99682 | 24: 20929473 | 99693 | 24: 21272688 | 99695 | 24: 21306399 |
| 99696 | 24: 21375203 | 99697 | 24: 21376904 | 99698 | 24: 21376940 | 99699 | 24: 21376967 | 99700 | 24: 21376972 |
| 99701 | 24: 21378281 | 99702 | 24: 21378321 | 99703 | 24: 21378352 | 99704 | 24: 21378382 | 99705 | 24: 21378383 |
| 99706 | 24: 21378384 | 99708 | 24: 21378440 | 99709 | 24: 21378445 | 99711 | 24: 21395177 | 99712 | 24: 21396591 |
| 99716 | 24: 21432058 | 99717 | 24: 21452284 | 99718 | 24: 21456850 | 99719 | 24: 21471419 | 99723 | 24: 21488223 |
| 99725 | 24: 21504166 | 99729 | 24: 21560026 | 99730 | 24: 21560060 | 99732 | 24: 21564234 | 99733 | 24: 21587057 |
| 99734 | 24: 21590358 | 99735 | 24: 21595564 | 99736 | 24: 21598260 | 99737 | 24: 21605659 | 99738 | 24: 21620362 |
| 99739 | 24: 21667119 | 99740 | 24: 21721258 | 99742 | 24: 21765426 | 99754 | 24: 21940194 | 99756 | 24: 21971534 |
| 99762 | 24: 22139528 | 99769 | 24: 22384770 | 99777 | 24: 22507325 | 99779 | 24: 22552252 | 99786 | 24: 22901733 |
| 99787 | 24: 22901743 | 99788 | 24: 22901792 | 99789 | 24: 22901810 | 99790 | 24: 22901819 | 99795 | 24: 23015622 |
| 99797 | 24: 23048740 | 99798 | 24: 23048839 | 99800 | 24: 23138496 | 99808 | 24: 23304844 | 99809 | 24: 23357754 |
| 99812 | 24: 23476124 | 99840 | 24: 24470786 | 99848 | 24: 24601724 | 99850 | 24: 24651791 | 99856 | 24: 24861946 |
| 99858 | 24: 24942299 | 99859 | 24: 24968711 | 99864 | 24: 25055928 | 99867 | 24: 25166673 | 99870 | 24: 25259209 |
| 99871 | 24: 25291130 | 99875 | 24: 25393251 | 99880 | 24: 25549639 | 99881 | 24: 25550670 | 99882 | 24: 25554087 |
| 99883 | 24: 25555252 | 99884 | 24: 25586246 | 99887 | 24: 25655955 | 99891 | 24: 25728080 | 99892 | 24: 25730700 |
| 99893 | 24: 25732798 | 99895 | 24: 25765374 | 99901 | 24: 25840462 | 99928 | 24: 26414878 | 99929 | 24: 26414942 |
| 99939 | 24: 26752400 | 99960 | 24: 27335211 | 99976 | 24: 27659643 | 99977 | 24: 27671338 | 99978 | 24: 27707920 |
| 99987 | 24: 27976475 | 99992 | 24: 28073538 | 10001 | 24: 28717465 | 10002 | 24: 28990565 | 10002 | 24: 28990603 |
| 10002 | 24: 28990620 | 10003 | 24: 29071264 | 10003 | 24: 29205694 | 10003 | 24: 29294106 | 10004 | 24: 29309403 |
| 10005 | 24: 29593728 | 10005 | 24: 29785127 | 10006 | 24: 30022971 | 10006 | 24: 30028664 | 10006 | 24: 30028736 |
| 10007 | 24: 30132653 | 10008 | 24: 30418113 | 10010 | 24: 30736100 | 10010 | 24: 30756120 | 10010 | 24: 30756121 |
| 10011 | 24: 31302891 | 10011 | 24: 31387621 | 10012 | 24: 31602471 | 10014 | 24: 31910298 | 10015 | 24: 32159647 |
| 10015 | 24: 32159730 | 10016 | 24: 32335210 | 10017 | 24: 32561115 | 10017 | 24: 32732872 | 10017 | 24: 32732892 |
| 10018 | 24: 32732952 | 10018 | 24: 32735243 | 10018 | 24: 32769877 | 10018 | 24: 32810095 | 10018 | 24: 32824750 |
| 10018 | 24: 32826968 | 10019 | 24: 32827828 | 10019 | 24: 32830047 | 10019 | 24: 32830055 | 10019 | 24: 32840723 |
| 10019 | 24: 32857855 | 10019 | 24: 32868393 | 10019 | 24: 32875942 | 10020 | 24: 32894247 | 10020 | 24: 33166170 |
| 10021 | 24: 33227163 | 10021 | 24: 33227191 | 10023 | 24: 33780521 | 10025 | 24: 34326602 | 10025 | 24: 34364918 |
| 10026 | 24: 34614458 | 10026 | 24: 34760712 | 10026 | 24: 34762120 | 10027 | 24: 34813345 | 10028 | 24: 35185894 |
| 10028 | 24: 35185970 | 10029 | 24: 35309422 | 10029 | 24: 35335089 | 10029 | 24: 35472794 | 10032 | 24: 35837548 |
| 10034 | 24: 36589347 | 10034 | 24: 36589477 | 10035 | 24: 36835198 | 10035 | 24: 36988061 | 10036 | 24: 37134995 |
| 10036 | 24: 37239650 | 10036 | 24: 37280274 | 10036 | 24: 37304503 | 10036 | 24: 37306947 | 10037 | 24: 37417740 |
| 10038 | 24: 37592997 | 10038 | 24: 37706524 | 10039 | 24: 37931336 | 10040 | 24: 38475906 | 10040 | 24: 38609603 |
| 10041 | 24: 38809089 | 10042 | 24: 38949417 | 10043 | 24: 39200562 | 10043 | 24: 39308113 | 10043 | 24: 39456209 |
| 10043 | 24: 39464085 | 10044 | 24: 39492963 | 10044 | 24: 39687343 | 10045 | 24: 39707191 | 10045 | 24: 39897863 |
| 10045 | 24: 39902640 | 10046 | 24: 39906046 | 10046 | 24: 39906190 | 10046 | 24: 39913847 | 10047 | 24: 40288339 |
| 10049 | 24: 40342848 | 10049 | 24: 40352389 | 10049 | 24: 40395247 | 10049 | 24: 40408675 | 10050 | 24: 40466974 |
| 10050 | 24: 40545027 | 10050 | 24: 40573682 | 10051 | 24: 40583210 | 10051 | 24: 40583246 | 10051 | 24: 40587358 |
| 10051 | 24: 40746951 | 10052 | 24: 40756834 | 10052 | 24: 40764626 | 10052 | 24: 40781770 | 10052 | 24: 40798538 |
| 10052 | 24: 40803087 | 10053 | 24: 40974776 | 10054 | 24: 41093545 | 10054 | 24: 41180993 | 10054 | 24: 41204807 |
| 10056 | 24: 41805610 | 10057 | 24: 41935257 | 10057 | 24: 41944725 | 10057 | 24: 42049855 | 10058 | 24: 42141779 |
| 10058 | 24: 42231198 | 10058 | 24: 42251623 | 10058 | 24: 42272285 | 10058 | 24: 42276093 | 10059 | 24: 42324465 |
| 10059 | 24: 42659053 | 10060 | 24: 42659123 | 10060 | 24: 42659180 | 10060 | 24: 42659202 | 10060 | 24: 42709614 |
| 10060 | 24: 42721124 | 10060 | 24: 42724162 | 10060 | 24: 42725838 | 10060 | 24: 42726075 | 10060 | 24: 42727665 |
| 10061 | 24: 42768635 | 10061 | 24: 42779119 | 10061 | 24: 42816514 | 10061 | 24: 42881112 | 10063 | 24: 43435997 |
| 10063 | 24: 43500003 | 10064 | 24: 44069431 | 10065 | 24: 44298375 | 10066 | 24: 44375642 | 10066 | 24: 44396418 |
| 10066 | 24: 44471971 | 10067 | 24: 44658910 | 10067 | 24: 44735016 | 10068 | 24: 44775120 | 10068 | 24: 44792435 |
| 10069 | 24: 45080627 | 10070 | 24: 45199003 | 10070 | 24: 45243583 | 10071 | 24: 45357351 | 10071 | 24: 45461858 |
| 10071 | 24: 45489006 | 10071 | 24: 45499596 | 10072 | 24: 45501942 | 10072 | 24: 45537440 | 10072 | 24: 45683209 |
| 10073 | 24: 45738887 | 10073 | 24: 45745474 | 10073 | 24: 45809276 | 10073 | 24: 45824753 | 10074 | 24: 45922636 |
| 10075 | 24: 45923194 | 10075 | 24: 46005539 | 10077 | 24: 46107365 | 10077 | 24: 46128800 | 10077 | 24: 46131144 |
| 10077 | 24: 46200299 | 10078 | 24: 46293419 | 10078 | 24: 46339304 | 10078 | 24: 46339449 | 10078 | 24: 46343493 |
| 10078 | 24: 46349257 | 10079 | 24: 46658398 | 10080 | 24: 46735235 | 10080 | 24: 46748616 | 10080 | 24: 46860941 |
| 10080 | 24: 46863126 | 10080 | 24: 46872067 | 10081 | 24: 46872779 | 10081 | 24: 46872783 | 10081 | 24: 46872835 |
| 10081 | 24: 46874235 | 10082 | 24: 47186952 | 10083 | 24: 47493053 | 10083 | 24: 47504673 | 10083 | 24: 47575681 |
| 10084 | 24: 47792768 | 10084 | 24: 47861526 | 10085 | 24: 47942540 | 10086 | 24: 48132259 | 10086 | 24: 48283076 |
| 10087 | 24: 48398019 | 10087 | 24: 48400794 | 10088 | 24: 48949533 | 10089 | 24: 49153895 | 10089 | 24: 49213874 |
| 10089 | 24: 49220538 | 10090 | 24: 49271697 | 10090 | 24: 49386058 | 10090 | 24: 49461650 | 10091 | 24: 49808792 |
| 10092 | 24: 49949844 | 10092 | 24: 49951357 | 10092 | 24: 49954724 | 10093 | 24: 50278508 | 10093 | 24: 50323489 |
| 10093 | 24: 50330708 | 10094 | 24: 50485176 | 10095 | 24: 50664362 | 10095 | 24: 50886055 | 10096 | 24: 51077466 |
| 10096 | 24: 51079480 | 10098 | 24: 51488836 | 10101 | 24: 51956661 | 10101 | 24: 52119070 | 10102 | 24: 52251430 |
| 10102 | 24: 52257462 | 10102 | 24: 52257497 | 10104 | 24: 52725727 | 10105 | 24: 53039441 | 10105 | 24: 53060437 |
| 10106 | 24: 53471017 | 10107 | 24: 53476039 | 10107 | 24: 53480782 | 10107 | 24: 53485369 | 10107 | 24: 53489339 |
| 10108 | 24: 53695135 | 10109 | 24: 54141567 | 10110 | 24: 54171685 | 10110 | 24: 54227463 | 10111 | 24: 54361200 |
| 10111 | 24: 54366075 | 10112 | 24: 54596604 | 10112 | 24: 54605333 | 10113 | 24: 54678133 | 10113 | 24: 54772296 |
| 10114 | 24: 54837850 | 10114 | 24: 54844868 | 10114 | 24: 54868156 | 10116 | 24: 55295472 | 10116 | 24: 55304369 |
| 10117 | 24: 55700714 | 10118 | 24: 55732464 | 10118 | 24: 55736959 | 10118 | 24: 55739826 | 10118 | 24: 55923426 |
| 10119 | 24: 56024340 | 10119 | 24: 56024373 | 10119 | 24: 56024406 | 10119 | 24: 56098690 | 10119 | 24: 56102284 |
| 10119 | 24: 56102647 | 10120 | 24: 56102653 | 10120 | 24: 56102851 | 10122 | 24: 56688810 | 10122 | 24: 56746296 |
| 10122 | 24: 56746319 | 10123 | 24: 56797648 | 10123 | 24: 56813204 | 10125 | 24: 57240950 | 10125 | 24: 57371376 |
| 10125 | 24: 57399764 | 10126 | 24: 57412925 | 10126 | 24: 57552725 | 10127 | 24: 57558999 | 10127 | 24: 57626126 |
| 10128 | 24: 57747906 | 10128 | 24: 57797863 | 10129 | 24: 58058760 | 10129 | 24: 58124009 | 10131 | 24: 58425089 |
| 10132 | 24: 58468125 | 10132 | 24: 58577110 | 10132 | 24: 58601846 | 10134 | 24: 58910690 | 10135 | 24: 59164671 |
| 10135 | 24: 59196985 | 10136 | 24: 59421526 | 10136 | 24: 59468734 | 10138 | 24: 59829538 | 10142 | 24: 60586093 |
| 10142 | 24: 60623865 | 10142 | 24: 60632706 | 10144 | 24: 60989011 | 10144 | 24: 61029712 | 10144 | 24: 61052179 |
| 10144 | 24: 61119884 | 10145 | 24: 61260912 | 10146 | 24: 61463290 | 10146 | 24: 61466635 | 10146 | 24: 61477326 |
| 10146 | 24: 61528992 | 10147 | 24: 61571637 | 10147 | 24: 61692816 | 10148 | 24: 61746382 | 10150 | 25: 121555 |
| 10150 | 25: 127561 | 10151 | 25: 343439 | 10151 | 25: 373190 | 10152 | 25: 376453 | 10152 | 25: 424297 |
| 10152 | 25: 567175 | 10152 | 25: 572006 | 10152 | 25: 670011 | 10152 | 25: 670169 | 10154 | 25: 950550 |
| 10154 | 25: 1045088 | 10154 | 25: 1087084 | 10154 | 25: 1134920 | 10155 | 25: 1139788 | 10155 | 25: 1177714 |
| 10155 | 25: 1296800 | 10155 | 25: 1324226 | 10156 | 25: 1352107 | 10156 | 25: 1353932 | 10156 | 25: 1354851 |
| 10156 | 25: 1364554 | 10156 | 25: 1364583 | 10156 | 25: 1365638 | 10157 | 25: 1405987 | 10157 | 25: 1476171 |
| 10157 | 25: 1478625 | 10158 | 25: 1649137 | 10158 | 25: 1775261 | 10158 | 25: 1777637 | 10159 | 25: 1777709 |
| 10159 | 25: 1777710 | 10159 | 25: 1777721 | 10159 | 25: 1783080 | 10159 | 25: 1790972 | 10159 | 25: 1791028 |
| 10159 | 25: 1808653 | 10159 | 25: 1818249 | 10160 | 25: 1835147 | 10160 | 25: 1862652 | 10160 | 25: 1883004 |
| 10160 | 25: 1966690 | 10161 | 25: 2003071 | 10162 | 25: 2200434 | 10162 | 25: 2247195 | 10165 | 25: 2376504 |
| 10166 | 25: 2422717 | 10166 | 25: 2477784 | 10166 | 25: 2479395 | 10166 | 25: 2479688 | 10166 | 25: 2479754 |
| 10167 | 25: 2537288 | 10167 | 25: 2538507 | 10167 | 25: 2540535 | 10167 | 25: 2547137 | 10167 | 25: 2548630 |
| 10167 | 25: 2552738 | 10167 | 25: 2553655 | 10167 | 25: 2555768 | 10167 | 25: 2560887 | 10167 | 25: 2561123 |
| 10168 | 25: 2563116 | 10168 | 25: 2564127 | 10168 | 25: 2565592 | 10168 | 25: 2568326 | 10168 | 25: 2573673 |
| 10168 | 25: 2575681 | 10168 | 25: 2578879 | 10168 | 25: 2580437 | 10168 | 25: 2581459 | 10169 | 25: 2582592 |
| 10169 | 25: 2585032 | 10169 | 25: 2586441 | 10170 | 25: 2681110 | 10170 | 25: 2681129 | 10170 | 25: 2687754 |
| 10170 | 25: 2695684 | 10170 | 25: 2695701 | 10170 | 25: 2695795 | 10170 | 25: 2695845 | 10171 | 25: 2695937 |
| 10171 | 25: 2696055 | 10171 | 25: 2696181 | 10171 | 25: 2696410 | 10171 | 25: 2698504 | 10171 | 25: 2699035 |
| 10171 | 25: 2699105 | 10171 | 25: 2739170 | 10171 | 25: 2740282 | 10171 | 25: 2740291 | 10172 | 25: 2743006 |
| 10172 | 25: 2743075 | 10172 | 25: 2743261 | 10172 | 25: 2743406 | 10172 | 25: 2749450 | 10172 | 25: 2762252 |
| 10172 | 25: 2762309 | 10172 | 25: 2775821 | 10173 | 25: 2819065 | 10173 | 25: 2819396 | 10173 | 25: 2819471 |
| 10173 | 25: 2819522 | 10173 | 25: 2819528 | 10173 | 25: 2819546 | 10173 | 25: 2819603 | 10175 | 25: 3210540 |
| 10175 | 25: 3310916 | 10176 | 25: 3363490 | 10176 | 25: 3426286 | 10176 | 25: 3477152 | 10177 | 25: 3524368 |
| 10177 | 25: 3529015 | 10177 | 25: 3534470 | 10177 | 25: 3534510 | 10177 | 25: 3554022 | 10178 | 25: 3581070 |
| 10178 | 25: 3588116 | 10178 | 25: 3595733 | 10178 | 25: 3604536 | 10178 | 25: 3606474 | 10178 | 25: 3625688 |
| 10178 | 25: 3630953 | 10178 | 25: 3640402 | 10178 | 25: 3650316 | 10178 | 25: 3656324 | 10179 | 25: 3701510 |
| 10179 | 25: 3830536 | 10179 | 25: 3851824 | 10180 | 25: 3903874 | 10180 | 25: 4088166 | 10181 | 25: 4278985 |
| 10181 | 25: 4319018 | 10185 | 25: 5024647 | 10186 | 25: 5186113 | 10186 | 25: 5326044 | 10191 | 25: 5967822 |
| 10192 | 25: 6012533 | 10193 | 25: 6175707 | 10193 | 25: 6231384 | 10195 | 25: 6788634 | 10196 | 25: 6838881 |
| 10196 | 25: 6862509 | 10196 | 25: 6862547 | 10198 | 25: 7313080 | 10198 | 25: 7484309 | 10198 | 25: 7517426 |
| 10199 | 25: 7734903 | 10199 | 25: 7840771 | 10199 | 25: 7840811 | 10200 | 25: 7884771 | 10200 | 25: 7905114 |
| 10201 | 25: 7941201 | 10201 | 25: 8064816 | 10202 | 25: 8388133 | 10202 | 25: 8511991 | 10204 | 25: 8788906 |
| 10204 | 25: 8844118 | 10205 | 25: 9197230 | 10206 | 25: 9300591 | 10206 | 25: 9365404 | 10206 | 25: 9378741 |
| 10206 | 25: 9385962 | 10207 | 25: 9502049 | 10207 | 25: 9643363 | 10207 | 25: 9687538 | 10208 | 25: 9776752 |
| 10208 | 25: 9843435 | 10209 | 25: 9993793 | 10209 | 25: 10007062 | 10210 | 25: 10139839 | 10210 | 25: 10198288 |
| 10210 | 25: 10227883 | 10210 | 25: 10272497 | 10211 | 25: 10365727 | 10211 | 25: 10541563 | 10211 | 25: 10541576 |
| 10212 | 25: 10547353 | 10213 | 25: 10832103 | 10213 | 25: 10836311 | 10213 | 25: 10948241 | 10213 | 25: 10951982 |
| 10214 | 25: 11012348 | 10214 | 25: 11110219 | 10214 | 25: 11111271 | 10215 | 25: 11138838 | 10216 | 25: 11274257 |
| 10216 | 25: 11436742 | 10216 | 25: 11437820 | 10217 | 25: 11447709 | 10217 | 25: 11455980 | 10217 | 25: 11549424 |
| 10217 | 25: 11549497 | 10217 | 25: 11570419 | 10220 | 25: 12214562 | 10221 | 25: 12401343 | 10223 | 25: 12943128 |
| 10224 | 25: 13251854 | 10224 | 25: 13339029 | 10224 | 25: 13345472 | 10225 | 25: 13388375 | 10226 | 25: 13601294 |
| 10227 | 25: 14013151 | 10228 | 25: 14073120 | 10228 | 25: 14201547 | 10229 | 25: 14377407 | 10229 | 25: 14409676 |
| 10229 | 25: 14448614 | 10229 | 25: 14511967 | 10230 | 25: 14519922 | 10230 | 25: 14583409 | 10231 | 25: 14703237 |
| 10231 | 25: 14862354 | 10232 | 25: 15092643 | 10233 | 25: 15276545 | 10233 | 25: 15276571 | 10233 | 25: 15370203 |
| 10235 | 25: 15564906 | 10235 | 25: 15633665 | 10235 | 25: 15633709 | 10235 | 25: 15633785 | 10236 | 25: 15912161 |
| 10236 | 25: 15942929 | 10236 | 25: 15943009 | 10237 | 25: 15996288 | 10237 | 25: 16093653 | 10238 | 25: 16219145 |
| 10238 | 25: 16288361 | 10238 | 25: 16288367 | 10239 | 25: 16429229 | 10240 | 25: 16494465 | 10242 | 25: 17127180 |
| 10243 | 25: 17439767 | 10243 | 25: 17446840 | 10244 | 25: 17474953 | 10244 | 25: 17475010 | 10244 | 25: 17483539 |
| 10244 | 25: 17488467 | 10245 | 25: 17838540 | 10246 | 25: 18019077 | 10246 | 25: 18091054 | 10247 | 25: 18283899 |
| 10247 | 25: 18285948 | 10248 | 25: 18730651 | 10248 | 25: 18747611 | 10248 | 25: 18760366 | 10248 | 25: 18798985 |
| 10249 | 25: 18976796 | 10250 | 25: 18988158 | 10253 | 25: 19881913 | 10253 | 25: 19929634 | 10254 | 25: 20012375 |
| 10254 | 25: 20043037 | 10254 | 25: 20085662 | 10255 | 25: 20338297 | 10257 | 25: 21049790 | 10257 | 25: 21139359 |
| 10258 | 25: 21196203 | 10258 | 25: 21260648 | 10258 | 25: 21317697 | 10259 | 25: 21318468 | 10259 | 25: 21327438 |
| 10259 | 25: 21329507 | 10259 | 25: 21405459 | 10260 | 25: 21506832 | 10260 | 25: 21524798 | 10260 | 25: 21532563 |
| 10262 | 25: 22103985 | 10264 | 25: 22376703 | 10264 | 25: 22394285 | 10264 | 25: 22420195 | 10265 | 25: 22670949 |
| 10268 | 25: 23399437 | 10269 | 25: 23750537 | 10271 | 25: 24033949 | 10271 | 25: 24132273 | 10272 | 25: 24242643 |
| 10272 | 25: 24361949 | 10274 | 25: 24619856 | 10274 | 25: 24768804 | 10275 | 25: 24829402 | 10275 | 25: 24857183 |
| 10275 | 25: 24902475 | 10275 | 25: 24915980 | 10275 | 25: 24918063 | 10275 | 25: 24918979 | 10276 | 25: 24918980 |
| 10276 | 25: 24920501 | 10277 | 25: 25159107 | 10277 | 25: 25226497 | 10278 | 25: 25231198 | 10278 | 25: 25252790 |
| 10278 | 25: 25319545 | 10278 | 25: 25319598 | 10278 | 25: 25327412 | 10278 | 25: 25327437 | 10278 | 25: 25355320 |
| 10278 | 25: 25355323 | 10279 | 25: 25370729 | 10280 | 25: 25518754 | 10280 | 25: 25644509 | 10280 | 25: 25729230 |
| 10280 | 25: 25764911 | 10281 | 25: 26048984 | 10283 | 25: 26491613 | 10283 | 25: 26491635 | 10283 | 25: 26491678 |
| 10283 | 25: 26491720 | 10283 | 25: 26491762 | 10284 | 25: 26492004 | 10284 | 25: 26492080 | 10284 | 25: 26492086 |
| 10284 | 25: 26492143 | 10284 | 25: 26493056 | 10284 | 25: 26493215 | 10284 | 25: 26493378 | 10285 | 25: 26873278 |
| 10285 | 25: 26879815 | 10286 | 25: 26957845 | 10286 | 25: 27074581 | 10286 | 25: 27078879 | 10286 | 25: 27079915 |
| 10286 | 25: 27081813 | 10288 | 25: 27471780 | 10288 | 25: 27505633 | 10288 | 25: 27529561 | 10288 | 25: 27546363 |
| 10288 | 25: 27577974 | 10288 | 25: 27610631 | 10289 | 25: 27614146 | 10289 | 25: 27621694 | 10289 | 25: 27623762 |
| 10289 | 25: 27634139 | 10289 | 25: 27647855 | 10289 | 25: 27651029 | 10289 | 25: 27654242 | 10289 | 25: 27671337 |
| 10289 | 25: 27672683 | 10289 | 25: 27677306 | 10290 | 25: 27724958 | 10290 | 25: 27728373 | 10290 | 25: 27731091 |
| 10290 | 25: 27734591 | 10290 | 25: 27737084 | 10290 | 25: 27743420 | 10290 | 25: 27747175 | 10291 | 25: 27747494 |
| 10291 | 25: 27747510 | 10291 | 25: 27748521 | 10291 | 25: 27749865 | 10291 | 25: 27749919 | 10291 | 25: 27749952 |
| 10291 | 25: 27749994 | 10291 | 25: 27750315 | 10291 | 25: 27750769 | 10291 | 25: 27751432 | 10292 | 25: 27790340 |
| 10293 | 25: 28024158 | 10293 | 25: 28057161 | 10293 | 25: 28188815 | 10294 | 25: 28274850 | 10294 | 25: 28376458 |
| 10295 | 25: 28539043 | 10295 | 25: 28760002 | 10297 | 25: 29158123 | 10298 | 25: 29422226 | 10298 | 25: 29507318 |
| 10299 | 25: 29574598 | 10299 | 25: 29653196 | 10300 | 25: 29711516 | 10301 | 25: 29924846 | 10302 | 25: 29960827 |
| 10302 | 25: 29983402 | 10302 | 25: 30108430 | 10303 | 25: 30188316 | 10306 | 25: 31059775 | 10308 | 25: 31397966 |
| 10308 | 25: 31397972 | 10312 | 25: 32101198 | 10312 | 25: 32115103 | 10312 | 25: 32122180 | 10313 | 25: 32225777 |
| 10314 | 25: 32620702 | 10314 | 25: 32636956 | 10315 | 25: 32651385 | 10315 | 25: 32784599 | 10315 | 25: 32785820 |
| 10315 | 25: 32838124 | 10316 | 25: 33064837 | 10316 | 25: 33068854 | 10317 | 25: 33278840 | 10317 | 25: 33325832 |
| 10318 | 25: 33472178 | 10319 | 25: 33614940 | 10319 | 25: 33626966 | 10320 | 25: 33862553 | 10320 | 25: 33918451 |
| 10320 | 25: 33918614 | 10321 | 25: 33933997 | 10321 | 25: 33986857 | 10322 | 25: 34165756 | 10322 | 25: 34324511 |
| 10323 | 25: 34373185 | 10323 | 25: 34380738 | 10323 | 25: 34390163 | 10323 | 25: 34415206 | 10323 | 25: 34416256 |
| 10323 | 25: 34423242 | 10323 | 25: 34430800 | 10324 | 25: 34512804 | 10324 | 25: 34523864 | 10324 | 25: 34541030 |
| 10324 | 25: 34607593 | 10324 | 25: 34616589 | 10325 | 25: 34627570 | 10325 | 25: 34729070 | 10326 | 25: 34979341 |
| 10327 | 25: 34987197 | 10327 | 25: 35035356 | 10327 | 25: 35047480 | 10327 | 25: 35162155 | 10328 | 25: 35220858 |
| 10328 | 25: 35223524 | 10328 | 25: 35224634 | 10329 | 25: 35416991 | 10329 | 25: 35510566 | 10329 | 25: 35538895 |
| 10329 | 25: 35539311 | 10330 | 25: 35574079 | 10330 | 25: 35596429 | 10330 | 25: 35632501 | 10331 | 25: 35695366 |
| 10331 | 25: 35695667 | 10331 | 25: 35695717 | 10331 | 25: 35696225 | 10331 | 25: 35696282 | 10331 | 25: 35697157 |
| 10331 | 25: 35723853 | 10331 | 25: 35726457 | 10331 | 25: 35737565 | 10332 | 25: 35767708 | 10332 | 25: 35774507 |
| 10332 | 25: 35775008 | 10332 | 25: 35784841 | 10332 | 25: 35868596 | 10333 | 25: 35874518 | 10333 | 25: 35930268 |
| 10333 | 25: 35960525 | 10334 | 25: 36192757 | 10334 | 25: 36214845 | 10334 | 25: 36214858 | 10334 | 25: 36215138 |
| 10335 | 25: 36215175 | 10335 | 25: 36215182 | 10335 | 25: 36215303 | 10335 | 25: 36215758 | 10335 | 25: 36215759 |
| 10335 | 25: 36216611 | 10335 | 25: 36216901 | 10335 | 25: 36216911 | 10335 | 25: 36217079 | 10335 | 25: 36217150 |
| 10336 | 25: 36217198 | 10336 | 25: 36217712 | 10336 | 25: 36217751 | 10336 | 25: 36225793 | 10336 | 25: 36225817 |
| 10336 | 25: 36246266 | 10336 | 25: 36249624 | 10337 | 25: 36344903 | 10337 | 25: 36350043 | 10337 | 25: 36360014 |
| 10337 | 25: 36450152 | 10338 | 25: 36450202 | 10338 | 25: 36512847 | 10338 | 25: 36531061 | 10338 | 25: 36535022 |
| 10338 | 25: 36538745 | 10338 | 25: 36584800 | 10339 | 25: 36811728 | 10339 | 25: 36971262 | 10339 | 25: 36978649 |
| 10340 | 25: 36985203 | 10340 | 25: 37031840 | 10340 | 25: 37034930 | 10340 | 25: 37043595 | 10340 | 25: 37046117 |
| 10340 | 25: 37060161 | 10340 | 25: 37061145 | 10341 | 25: 37136607 | 10341 | 25: 37136612 | 10341 | 25: 37138640 |
| 10341 | 25: 37138651 | 10341 | 25: 37138668 | 10341 | 25: 37149974 | 10342 | 25: 37184274 | 10342 | 25: 37205234 |
| 10342 | 25: 37227790 | 10342 | 25: 37241594 | 10342 | 25: 37242352 | 10342 | 25: 37242939 | 10343 | 25: 37242955 |
| 10343 | 25: 37242995 | 10343 | 25: 37243078 | 10343 | 25: 37252529 | 10343 | 25: 37356346 | 10344 | 25: 37389979 |
| 10346 | 25: 37729407 | 10346 | 25: 37784956 | 10347 | 25: 37964578 | 10348 | 25: 38114076 | 10348 | 25: 38114479 |
| 10348 | 25: 38117206 | 10348 | 25: 38118273 | 10348 | 25: 38126701 | 10348 | 25: 38166852 | 10348 | 25: 38190732 |
| 10349 | 25: 38227705 | 10349 | 25: 38233097 | 10349 | 25: 38300677 | 10349 | 25: 38300772 | 10349 | 25: 38300781 |
| 10350 | 25: 38363440 | 10350 | 25: 38394640 | 10350 | 25: 38394741 | 10350 | 25: 38489542 | 10352 | 25: 38856435 |
| 10352 | 25: 38930854 | 10353 | 25: 39028696 | 10353 | 25: 39045580 | 10354 | 25: 39300696 | 10354 | 25: 39317059 |
| 10354 | 25: 39342146 | 10354 | 25: 39345282 | 10354 | 25: 39449897 | 10355 | 25: 39552784 | 10357 | 25: 39918879 |
| 10359 | 25: 40240409 | 10359 | 25: 40240500 | 10360 | 25: 40328748 | 10360 | 25: 40396102 | 10360 | 25: 40553090 |
| 10361 | 25: 40614314 | 10362 | 25: 40720968 | 10362 | 25: 40758343 | 10363 | 25: 40876573 | 10363 | 25: 40901328 |
| 10363 | 25: 40903884 | 10363 | 25: 40948048 | 10364 | 25: 40954468 | 10364 | 25: 40956329 | 10364 | 25: 40960034 |
| 10364 | 25: 40960925 | 10364 | 25: 40979131 | 10364 | 25: 40982964 | 10364 | 25: 40991964 | 10364 | 25: 40998163 |
| 10364 | 25: 41007519 | 10365 | 25: 41009480 | 10365 | 25: 41012986 | 10365 | 25: 41014477 | 10365 | 25: 41015047 |
| 10365 | 25: 41022922 | 10365 | 25: 41028182 | 10365 | 25: 41031444 | 10365 | 25: 41033389 | 10365 | 25: 41034653 |
| 10365 | 25: 41039109 | 10366 | 25: 41046191 | 10366 | 25: 41066639 | 10366 | 25: 41070067 | 10366 | 25: 41074859 |
| 10367 | 25: 41373314 | 10367 | 25: 41416055 | 10367 | 25: 41479611 | 10367 | 25: 41496081 | 10368 | 25: 41719279 |
| 10368 | 25: 41758231 | 10369 | 25: 42027128 | 10369 | 25: 42031008 | 10370 | 25: 42287133 | 10370 | 25: 42290491 |
| 10370 | 25: 42292572 | 10371 | 26: 840412 | 10372 | 26: 1046523 | 10373 | 26: 1202224 | 10374 | 26: 1527005 |
| 10375 | 26: 1774152 | 10375 | 26: 1837613 | 10375 | 26: 1837679 | 10375 | 26: 1837715 | 10378 | 26: 2789785 |
| 10379 | 26: 2795689 | 10379 | 26: 2807861 | 10379 | 26: 2809033 | 10379 | 26: 2810402 | 10379 | 26: 2810457 |
| 10379 | 26: 2810725 | 10379 | 26: 2811451 | 10379 | 26: 2811491 | 10379 | 26: 2811596 | 10379 | 26: 2811603 |
| 10380 | 26: 2811953 | 10380 | 26: 2812213 | 10380 | 26: 2812496 | 10380 | 26: 2834801 | 10380 | 26: 2836372 |
| 10380 | 26: 2838470 | 10380 | 26: 2839812 | 10380 | 26: 2843878 | 10380 | 26: 2847631 | 10380 | 26: 2853005 |
| 10381 | 26: 2869288 | 10381 | 26: 2912970 | 10382 | 26: 3233006 | 10382 | 26: 3276739 | 10382 | 26: 3276794 |
| 10382 | 26: 3347635 | 10382 | 26: 3347724 | 10385 | 26: 3975478 | 10387 | 26: 4592577 | 10389 | 26: 4967710 |
| 10389 | 26: 5039459 | 10390 | 26: 5408963 | 10395 | 26: 6941203 | 10395 | 26: 6944017 | 10396 | 26: 7431785 |
| 10396 | 26: 7456216 | 10396 | 26: 7484390 | 10397 | 26: 7488314 | 10397 | 26: 7495226 | 10397 | 26: 7510638 |
| 10397 | 26: 7591687 | 10397 | 26: 7696472 | 10398 | 26: 7696485 | 10398 | 26: 7727559 | 10399 | 26: 8027593 |
| 10399 | 26: 8048518 | 10400 | 26: 8204339 | 10401 | 26: 8516699 | 10401 | 26: 8538464 | 10402 | 26: 8757164 |
| 10403 | 26: 8943593 | 10403 | 26: 8960699 | 10404 | 26: 9137009 | 10405 | 26: 9666870 | 10406 | 26: 9801561 |
| 10407 | 26: 10180467 | 10407 | 26: 10180516 | 10408 | 26: 10195205 | 10408 | 26: 10195875 | 10408 | 26: 10196016 |
| 10408 | 26: 10197224 | 10408 | 26: 10201006 | 10408 | 26: 10203129 | 10408 | 26: 10208363 | 10408 | 26: 10212949 |
| 10408 | 26: 10214107 | 10408 | 26: 10221720 | 10409 | 26: 10302490 | 10409 | 26: 10305675 | 10410 | 26: 10616738 |
| 10411 | 26: 10731742 | 10412 | 26: 11167367 | 10413 | 26: 11511468 | 10414 | 26: 11702998 | 10414 | 26: 11703000 |
| 10414 | 26: 11703003 | 10414 | 26: 11703009 | 10415 | 26: 11703013 | 10415 | 26: 11703046 | 10415 | 26: 11703059 |
| 10415 | 26: 11703143 | 10417 | 26: 12409311 | 10417 | 26: 12493226 | 10417 | 26: 12493314 | 10417 | 26: 12576243 |
| 10417 | 26: 12663124 | 10418 | 26: 12857727 | 10419 | 26: 12905617 | 10419 | 26: 12936955 | 10419 | 26: 13005236 |
| 10420 | 26: 13185986 | 10420 | 26: 13213326 | 10421 | 26: 13237530 | 10421 | 26: 13242646 | 10421 | 26: 13253767 |
| 10422 | 26: 13348439 | 10422 | 26: 13439428 | 10422 | 26: 13487765 | 10423 | 26: 13487832 | 10423 | 26: 13628978 |
| 10423 | 26: 13632894 | 10423 | 26: 13666033 | 10424 | 26: 13818460 | 10424 | 26: 13935413 | 10425 | 26: 14204588 |
| 10425 | 26: 14204609 | 10427 | 26: 14670368 | 10427 | 26: 14688221 | 10427 | 26: 14729665 | 10427 | 26: 14889689 |
| 10427 | 26: 14890394 | 10428 | 26: 14896553 | 10428 | 26: 15033159 | 10429 | 26: 15470094 | 10431 | 26: 15856645 |
| 10431 | 26: 15856778 | 10431 | 26: 15865811 | 10431 | 26: 15866192 | 10431 | 26: 15867927 | 10431 | 26: 15867943 |
| 10431 | 26: 15876906 | 10434 | 26: 16752906 | 10434 | 26: 16926415 | 10434 | 26: 16926501 | 10435 | 26: 16951340 |
| 10435 | 26: 17033041 | 10435 | 26: 17041251 | 10435 | 26: 17100094 | 10436 | 26: 17127186 | 10436 | 26: 17156417 |
| 10436 | 26: 17197835 | 10436 | 26: 17325459 | 10437 | 26: 17482702 | 10437 | 26: 17539828 | 10438 | 26: 17628730 |
| 10438 | 26: 17628780 | 10438 | 26: 17685991 | 10438 | 26: 17691480 | 10438 | 26: 17691492 | 10439 | 26: 17746468 |
| 10439 | 26: 17781937 | 10439 | 26: 17879952 | 10440 | 26: 17928505 | 10440 | 26: 17965085 | 10441 | 26: 18209480 |
| 10441 | 26: 18211289 | 10441 | 26: 18355673 | 10442 | 26: 18490628 | 10442 | 26: 18494442 | 10442 | 26: 18521176 |
| 10442 | 26: 18528824 | 10443 | 26: 18651237 | 10443 | 26: 18695230 | 10443 | 26: 18702491 | 10443 | 26: 18728512 |
| 10443 | 26: 18740541 | 10444 | 26: 18782784 | 10444 | 26: 18869630 | 10444 | 26: 18885108 | 10444 | 26: 18907964 |
| 10444 | 26: 18929393 | 10445 | 26: 18970293 | 10445 | 26: 19009189 | 10446 | 26: 19238259 | 10447 | 26: 19438464 |
| 10447 | 26: 19452547 | 10447 | 26: 19494159 | 10448 | 26: 19510054 | 10448 | 26: 19547291 | 10448 | 26: 19597530 |
| 10449 | 26: 20104927 | 10449 | 26: 20143627 | 10451 | 26: 20594269 | 10452 | 26: 21094234 | 10453 | 26: 21239671 |
| 10453 | 26: 21254361 | 10456 | 26: 21784164 | 10456 | 26: 21862417 | 10456 | 26: 21918010 | 10456 | 26: 21964449 |
| 10456 | 26: 22002686 | 10457 | 26: 22058857 | 10457 | 26: 22063661 | 10457 | 26: 22251251 | 10458 | 26: 22554517 |
| 10458 | 26: 22581259 | 10458 | 26: 22606617 | 10458 | 26: 22658743 | 10459 | 26: 22659438 | 10461 | 26: 23328557 |
| 10461 | 26: 23398930 | 10461 | 26: 23503177 | 10462 | 26: 23741250 | 10463 | 26: 24028517 | 10463 | 26: 24127485 |
| 10463 | 26: 24135884 | 10464 | 26: 24188588 | 10464 | 26: 24239524 | 10464 | 26: 24286733 | 10464 | 26: 24287516 |
| 10464 | 26: 24310631 | 10465 | 26: 24590237 | 10466 | 26: 24630200 | 10466 | 26: 24764833 | 10466 | 26: 24781597 |
| 10466 | 26: 24818702 | 10467 | 26: 24819776 | 10467 | 26: 24820679 | 10467 | 26: 24823044 | 10467 | 26: 24826317 |
| 10467 | 26: 24829772 | 10467 | 26: 24831082 | 10467 | 26: 24831934 | 10467 | 26: 24850967 | 10468 | 26: 24867806 |
| 10468 | 26: 24877750 | 10468 | 26: 24920605 | 10470 | 26: 25371117 | 10470 | 26: 25377110 | 10471 | 26: 25408794 |
| 10471 | 26: 25438245 | 10472 | 26: 25560669 | 10472 | 26: 25586394 | 10472 | 26: 25601218 | 10472 | 26: 25624158 |
| 10472 | 26: 25628586 | 10472 | 26: 25632049 | 10472 | 26: 25636772 | 10472 | 26: 25637820 | 10472 | 26: 25640830 |
| 10473 | 26: 25643571 | 10473 | 26: 25645847 | 10473 | 26: 25646718 | 10473 | 26: 25649902 | 10473 | 26: 25653351 |
| 10473 | 26: 25664984 | 10473 | 26: 25690676 | 10474 | 26: 25738398 | 10474 | 26: 25884432 | 10474 | 26: 25905471 |
| 10474 | 26: 25923067 | 10474 | 26: 25924666 | 10475 | 26: 26022373 | 10475 | 26: 26108845 | 10476 | 26: 26209053 |
| 10476 | 26: 26211205 | 10477 | 26: 26461336 | 10479 | 26: 26855141 | 10480 | 26: 27069339 | 10482 | 26: 27558387 |
| 10482 | 26: 27572772 | 10483 | 26: 27736462 | 10483 | 26: 27736471 | 10483 | 26: 27736612 | 10485 | 26: 28197657 |
| 10487 | 26: 28950952 | 10487 | 26: 29088929 | 10488 | 26: 29186399 | 10489 | 26: 29513286 | 10491 | 26: 29986030 |
| 10491 | 26: 30197525 | 10491 | 26: 30197564 | 10491 | 26: 30197578 | 10492 | 26: 30197681 | 10493 | 26: 30889276 |
| 10493 | 26: 30892846 | 10494 | 26: 30950051 | 10495 | 26: 31271680 | 10495 | 26: 31311514 | 10495 | 26: 31323087 |
| 10496 | 26: 31350194 | 10496 | 26: 31368850 | 10496 | 26: 31379154 | 10496 | 26: 31411730 | 10496 | 26: 31483017 |
| 10497 | 26: 31801170 | 10499 | 26: 32178829 | 10499 | 26: 32210509 | 10500 | 26: 32484431 | 10500 | 26: 32527484 |
| 10501 | 26: 32691372 | 10501 | 26: 32756815 | 10502 | 26: 32879086 | 10503 | 26: 33122620 | 10504 | 26: 33282896 |
| 10504 | 26: 33328268 | 10504 | 26: 33359761 | 10504 | 26: 33380765 | 10504 | 26: 33400303 | 10505 | 26: 33526762 |
| 10505 | 26: 33530073 | 10505 | 26: 33560198 | 10506 | 26: 33837346 | 10506 | 26: 33901952 | 10507 | 26: 33914479 |
| 10507 | 26: 33922739 | 10508 | 26: 34087101 | 10508 | 26: 34109859 | 10508 | 26: 34125393 | 10508 | 26: 34140428 |
| 10508 | 26: 34145240 | 10508 | 26: 34148000 | 10509 | 26: 34318487 | 10509 | 26: 34354045 | 10510 | 26: 34633936 |
| 10511 | 26: 34718279 | 10511 | 26: 34746698 | 10511 | 26: 34746782 | 10511 | 26: 34746817 | 10511 | 26: 34746843 |
| 10511 | 26: 34746854 | 10511 | 26: 34746898 | 10511 | 26: 34755555 | 10512 | 26: 34829732 | 10512 | 26: 34832045 |
| 10512 | 26: 35009461 | 10513 | 26: 35045257 | 10513 | 26: 35119651 | 10513 | 26: 35152534 | 10514 | 26: 35387482 |
| 10514 | 26: 35446688 | 10515 | 26: 35473524 | 10515 | 26: 35703319 | 10515 | 26: 35730641 | 10515 | 26: 35730653 |
| 10515 | 26: 35730692 | 10515 | 26: 35730695 | 10516 | 26: 35894255 | 10518 | 26: 36442085 | 10518 | 26: 36465890 |
| 10518 | 26: 36466008 | 10519 | 26: 36782486 | 10520 | 26: 36868486 | 10520 | 26: 36875653 | 10520 | 26: 36881092 |
| 10520 | 26: 36895816 | 10520 | 26: 36899560 | 10520 | 26: 36899693 | 10520 | 26: 36955993 | 10520 | 26: 36966986 |
| 10521 | 26: 36968918 | 10521 | 26: 36968980 | 10521 | 26: 36969055 | 10522 | 26: 37095749 | 10523 | 26: 37812473 |
| 10524 | 26: 37879370 | 10524 | 26: 37908929 | 10524 | 26: 38110713 | 10526 | 26: 38674064 | 10527 | 26: 38851055 |
| 10527 | 26: 38854747 | 10527 | 26: 38875810 | 10527 | 26: 38877239 | 10527 | 26: 38881457 | 10528 | 26: 38992651 |
| 10528 | 26: 39006604 | 10529 | 26: 39098131 | 10529 | 26: 39189961 | 10529 | 26: 39196795 | 10529 | 26: 39202461 |
| 10530 | 26: 39305962 | 10530 | 26: 39426186 | 10530 | 26: 39426206 | 10530 | 26: 39426264 | 10530 | 26: 39485796 |
| 10531 | 26: 39494577 | 10531 | 26: 39507180 | 10531 | 26: 39538069 | 10531 | 26: 39573736 | 10532 | 26: 39783330 |
| 10532 | 26: 40032776 | 10533 | 26: 40040946 | 10533 | 26: 40070046 | 10534 | 26: 40441942 | 10535 | 26: 40786447 |
| 10536 | 26: 41019385 | 10536 | 26: 41076854 | 10536 | 26: 41096640 | 10537 | 26: 41390989 | 10539 | 26: 41918819 |
| 10539 | 26: 41926321 | 10540 | 26: 42008210 | 10540 | 26: 42024771 | 10540 | 26: 42049951 | 10540 | 26: 42090283 |
| 10540 | 26: 42092905 | 10541 | 26: 42109007 | 10541 | 26: 42309356 | 10541 | 26: 42330778 | 10542 | 26: 42350916 |
| 10543 | 26: 42642591 | 10543 | 26: 42660447 | 10543 | 26: 42757714 | 10544 | 26: 43041112 | 10545 | 26: 43062676 |
| 10546 | 26: 43622892 | 10547 | 26: 43740696 | 10547 | 26: 43816442 | 10548 | 26: 44064506 | 10548 | 26: 44083273 |
| 10548 | 26: 44083285 | 10549 | 26: 44083315 | 10549 | 26: 44083393 | 10549 | 26: 44083680 | 10550 | 26: 44255933 |
| 10550 | 26: 44404294 | 10550 | 26: 44481659 | 10551 | 26: 44674003 | 10551 | 26: 44723893 | 10552 | 26: 45008042 |
| 10553 | 26: 45209100 | 10553 | 26: 45264287 | 10554 | 26: 45324531 | 10555 | 26: 45412608 | 10556 | 26: 45540372 |
| 10556 | 26: 45540404 | 10556 | 26: 45540418 | 10556 | 26: 45540427 | 10556 | 26: 45542714 | 10556 | 26: 45565711 |
| 10557 | 26: 45729275 | 10558 | 26: 46112041 | 10559 | 26: 46315775 | 10559 | 26: 46317103 | 10559 | 26: 46317812 |
| 10559 | 26: 46318923 | 10560 | 26: 46353080 | 10560 | 26: 46421801 | 10560 | 26: 46452461 | 10560 | 26: 46466580 |
| 10560 | 26: 46471616 | 10560 | 26: 46479016 | 10561 | 26: 46559579 | 10563 | 26: 47028707 | 10564 | 26: 47333154 |
| 10564 | 26: 47361635 | 10564 | 26: 47427206 | 10565 | 26: 47512133 | 10565 | 26: 47512681 | 10565 | 26: 47615883 |
| 10566 | 26: 47650142 | 10566 | 26: 47772970 | 10569 | 26: 48408440 | 10569 | 26: 48479152 | 10571 | 26: 48865592 |
| 10571 | 26: 48866736 | 10571 | 26: 48968839 | 10571 | 26: 49004191 | 10574 | 26: 49515195 | 10574 | 26: 49563646 |
| 10575 | 26: 49563747 | 10579 | 26: 50590249 | 10579 | 26: 50709285 | 10579 | 26: 50733543 | 10580 | 26: 50733621 |
| 10580 | 26: 50823585 | 10580 | 26: 50825362 | 10580 | 26: 50830075 | 10581 | 26: 51009547 | 10581 | 26: 51009628 |
| 10581 | 26: 51127593 | 10582 | 26: 51175800 | 10583 | 26: 51422136 | 10584 | 26: 51748308 | 10584 | 26: 51784240 |
| 10584 | 26: 51824417 | 10584 | 26: 51837838 | 10584 | 26: 51856701 | 10585 | 26: 51990316 | 10586 | 27: 504917 |
| 10586 | 27: 504918 | 10586 | 27: 504943 | 10586 | 27: 622508 | 10587 | 27: 1220407 | 10588 | 27: 1285546 |
| 10588 | 27: 1330181 | 10588 | 27: 1355435 | 10589 | 27: 1355462 | 10589 | 27: 1362304 | 10589 | 27: 1362959 |
| 10589 | 27: 1375496 | 10589 | 27: 1376218 | 10589 | 27: 1382415 | 10589 | 27: 1384981 | 10589 | 27: 1385318 |
| 10589 | 27: 1385323 | 10589 | 27: 1385392 | 10590 | 27: 1385425 | 10590 | 27: 1385455 | 10590 | 27: 1395995 |
| 10590 | 27: 1401856 | 10590 | 27: 1401865 | 10590 | 27: 1405119 | 10590 | 27: 1407822 | 10590 | 27: 1453147 |
| 10591 | 27: 1475170 | 10593 | 27: 2200746 | 10593 | 27: 2232971 | 10594 | 27: 2375493 | 10595 | 27: 2508576 |
| 10597 | 27: 2988336 | 10600 | 27: 3776760 | 10600 | 27: 3787074 | 10600 | 27: 3788517 | 10600 | 27: 3790934 |
| 10600 | 27: 3805789 | 10602 | 27: 4234197 | 10604 | 27: 4820720 | 10604 | 27: 4843559 | 10605 | 27: 4942987 |
| 10605 | 27: 4946073 | 10605 | 27: 4962502 | 10605 | 27: 4964043 | 10605 | 27: 5085626 | 10606 | 27: 5098545 |
| 10606 | 27: 5158253 | 10606 | 27: 5391087 | 10607 | 27: 5392011 | 10607 | 27: 5395069 | 10607 | 27: 5396567 |
| 10607 | 27: 5399954 | 10607 | 27: 5401556 | 10607 | 27: 5402389 | 10607 | 27: 5403342 | 10607 | 27: 5404401 |
| 10607 | 27: 5418944 | 10608 | 27: 5421637 | 10608 | 27: 5428881 | 10608 | 27: 5430538 | 10608 | 27: 5470087 |
| 10608 | 27: 5478901 | 10608 | 27: 5582204 | 10609 | 27: 5587883 | 10609 | 27: 5611632 | 10609 | 27: 5792112 |
| 10610 | 27: 5827416 | 10610 | 27: 5974973 | 10610 | 27: 5988748 | 10610 | 27: 6025859 | 10610 | 27: 6032871 |
| 10611 | 27: 6060484 | 10611 | 27: 6087111 | 10614 | 27: 7631148 | 10614 | 27: 7653356 | 10614 | 27: 7733608 |
| 10615 | 27: 7750098 | 10615 | 27: 7770345 | 10615 | 27: 7791683 | 10615 | 27: 7807058 | 10615 | 27: 7813201 |
| 10616 | 27: 7920370 | 10616 | 27: 7920432 | 10616 | 27: 7920451 | 10616 | 27: 7958336 | 10617 | 27: 7975782 |
| 10617 | 27: 7980889 | 10617 | 27: 8132488 | 10617 | 27: 8191553 | 10618 | 27: 8289670 | 10618 | 27: 8359908 |
| 10618 | 27: 8470036 | 10618 | 27: 8476834 | 10619 | 27: 8575128 | 10619 | 27: 8612967 | 10623 | 27: 9145723 |
| 10624 | 27: 9202230 | 10624 | 27: 9202257 | 10624 | 27: 9202263 | 10624 | 27: 9202295 | 10625 | 27: 9488574 |
| 10630 | 27: 11255396 | 10630 | 27: 11262348 | 10632 | 27: 11669357 | 10632 | 27: 11736207 | 10634 | 27: 12235101 |
| 10634 | 27: 12270834 | 10637 | 27: 12976567 | 10637 | 27: 13162684 | 10638 | 27: 13319931 | 10639 | 27: 13521583 |
| 10640 | 27: 13703268 | 10640 | 27: 13763652 | 10641 | 27: 13769140 | 10641 | 27: 13864569 | 10645 | 27: 14058907 |
| 10645 | 27: 14089539 | 10646 | 27: 14337753 | 10647 | 27: 14505664 | 10647 | 27: 14619598 | 10650 | 27: 15114953 |
| 10650 | 27: 15209444 | 10651 | 27: 15342336 | 10651 | 27: 15374174 | 10652 | 27: 15616842 | 10653 | 27: 15679545 |
| 10653 | 27: 15679907 | 10653 | 27: 15679986 | 10653 | 27: 15679987 | 10653 | 27: 15724630 | 10655 | 27: 16091144 |
| 10656 | 27: 16231107 | 10656 | 27: 16231139 | 10656 | 27: 16250819 | 10656 | 27: 16262844 | 10657 | 27: 16289197 |
| 10657 | 27: 16289212 | 10657 | 27: 16310423 | 10657 | 27: 16311908 | 10657 | 27: 16311992 | 10658 | 27: 16448657 |
| 10658 | 27: 16475268 | 10658 | 27: 16527145 | 10658 | 27: 16527632 | 10659 | 27: 16545436 | 10659 | 27: 16626449 |
| 10660 | 27: 16876243 | 10661 | 27: 17114420 | 10663 | 27: 17756331 | 10663 | 27: 17825429 | 10664 | 27: 17900781 |
| 10668 | 27: 18743674 | 10670 | 27: 19184326 | 10670 | 27: 19226515 | 10671 | 27: 19413103 | 10672 | 27: 19615386 |
| 10672 | 27: 19628648 | 10672 | 27: 19638432 | 10672 | 27: 19643687 | 10672 | 27: 19643703 | 10672 | 27: 19656115 |
| 10673 | 27: 19709555 | 10673 | 27: 19715294 | 10673 | 27: 19715322 | 10673 | 27: 19743997 | 10673 | 27: 19759721 |
| 10674 | 27: 19916100 | 10675 | 27: 20275887 | 10676 | 27: 20403248 | 10677 | 27: 21028770 | 10679 | 27: 21603965 |
| 10680 | 27: 21736051 | 10681 | 27: 21968621 | 10682 | 27: 21988370 | 10682 | 27: 21988471 | 10682 | 27: 22006706 |
| 10683 | 27: 22555224 | 10686 | 27: 23217717 | 10686 | 27: 23219768 | 10686 | 27: 23235008 | 10686 | 27: 23291834 |
| 10688 | 27: 23591760 | 10688 | 27: 23763903 | 10689 | 27: 23820540 | 10689 | 27: 23900777 | 10690 | 27: 23960140 |
| 10690 | 27: 23969325 | 10690 | 27: 23997295 | 10690 | 27: 24130313 | 10691 | 27: 24151993 | 10691 | 27: 24152004 |
| 10691 | 27: 24203304 | 10691 | 27: 24203421 | 10691 | 27: 24222816 | 10692 | 27: 24521008 | 10693 | 27: 24725230 |
| 10694 | 27: 24839161 | 10694 | 27: 24839261 | 10696 | 27: 25252926 | 10696 | 27: 25328242 | 10697 | 27: 25416389 |
| 10698 | 27: 25562534 | 10698 | 27: 25566427 | 10698 | 27: 25728403 | 10699 | 27: 26089588 | 10700 | 27: 26265901 |
| 10700 | 27: 26306516 | 10700 | 27: 26385003 | 10701 | 27: 26490500 | 10702 | 27: 26581172 | 10702 | 27: 26586255 |
| 10702 | 27: 26593729 | 10703 | 27: 26832000 | 10704 | 27: 26846204 | 10704 | 27: 26878354 | 10704 | 27: 26886076 |
| 10704 | 27: 26894469 | 10704 | 27: 26904559 | 10704 | 27: 26906127 | 10704 | 27: 26924212 | 10706 | 27: 27222640 |
| 10706 | 27: 27223927 | 10706 | 27: 27223929 | 10706 | 27: 27224038 | 10706 | 27: 27224048 | 10706 | 27: 27224849 |
| 10706 | 27: 27224850 | 10708 | 27: 27637602 | 10709 | 27: 27970577 | 10710 | 27: 28171888 | 10713 | 27: 28946541 |
| 10714 | 27: 29252315 | 10715 | 27: 29278521 | 10715 | 27: 29296483 | 10715 | 27: 29312772 | 10715 | 27: 29327883 |
| 10715 | 27: 29327914 | 10716 | 27: 29327998 | 10718 | 27: 30202829 | 10719 | 27: 30202912 | 10719 | 27: 30226393 |
| 10719 | 27: 30244634 | 10722 | 27: 31002073 | 10722 | 27: 31002095 | 10722 | 27: 31002129 | 10723 | 27: 31078802 |
| 10724 | 27: 31274065 | 10726 | 27: 31729965 | 10726 | 27: 31841660 | 10726 | 27: 31841704 | 10727 | 27: 31897740 |
| 10729 | 27: 32226765 | 10729 | 27: 32254312 | 10729 | 27: 32265770 | 10730 | 27: 32268130 | 10730 | 27: 32276493 |
| 10731 | 27: 32518470 | 10731 | 27: 32518472 | 10731 | 27: 32518552 | 10731 | 27: 32583194 | 10732 | 27: 32727516 |
| 10732 | 27: 32727582 | 10732 | 27: 32727603 | 10732 | 27: 32727620 | 10732 | 27: 32778474 | 10732 | 27: 32793041 |
| 10733 | 27: 32802126 | 10734 | 27: 33001000 | 10734 | 27: 33009928 | 10734 | 27: 33086268 | 10735 | 27: 33233127 |
| 10735 | 27: 33265366 | 10735 | 27: 33272590 | 10736 | 27: 33421612 | 10736 | 27: 33629831 | 10736 | 27: 33633260 |
| 10737 | 27: 33872186 | 10737 | 27: 33879901 | 10737 | 27: 33913097 | 10738 | 27: 34127235 | 10739 | 27: 34635911 |
| 10740 | 27: 34637563 | 10740 | 27: 34752720 | 10741 | 27: 34981815 | 10741 | 27: 35014810 | 10741 | 27: 35015975 |
| 10741 | 27: 35016903 | 10741 | 27: 35018328 | 10741 | 27: 35019984 | 10741 | 27: 35021367 | 10742 | 27: 35026180 |
| 10742 | 27: 35032183 | 10742 | 27: 35042729 | 10742 | 27: 35045715 | 10742 | 27: 35047820 | 10742 | 27: 35052712 |
| 10742 | 27: 35053785 | 10742 | 27: 35054739 | 10742 | 27: 35058765 | 10743 | 27: 35060134 | 10743 | 27: 35064362 |
| 10743 | 27: 35065072 | 10743 | 27: 35072059 | 10743 | 27: 35075103 | 10743 | 27: 35079318 | 10743 | 27: 35080042 |
| 10743 | 27: 35098005 | 10744 | 27: 35103784 | 10744 | 27: 35121417 | 10744 | 27: 35122313 | 10744 | 27: 35128376 |
| 10744 | 27: 35138175 | 10744 | 27: 35184790 | 10747 | 27: 35555630 | 10747 | 27: 35599360 | 10748 | 27: 35782254 |
| 10748 | 27: 35824524 | 10748 | 27: 35824606 | 10749 | 27: 35975385 | 10749 | 27: 35978336 | 10750 | 27: 36138112 |
| 10750 | 27: 36216370 | 10752 | 27: 36576641 | 10753 | 27: 36751396 | 10753 | 27: 36906500 | 10753 | 27: 37106071 |
| 10754 | 27: 37127877 | 10754 | 27: 37172084 | 10754 | 27: 37192870 | 10754 | 27: 37195004 | 10754 | 27: 37274132 |
| 10755 | 27: 37275861 | 10755 | 27: 37293040 | 10755 | 27: 37297500 | 10756 | 27: 37374389 | 10756 | 27: 37486971 |
| 10756 | 27: 37493520 | 10756 | 27: 37521766 | 10757 | 27: 37573787 | 10757 | 27: 37620840 | 10757 | 27: 37630299 |
| 10757 | 27: 37639061 | 10758 | 27: 37746356 | 10759 | 27: 37827637 | 10759 | 27: 37864645 | 10760 | 27: 38151557 |
| 10760 | 27: 38168061 | 10761 | 27: 38327204 | 10761 | 27: 38432520 | 10762 | 27: 38491615 | 10763 | 27: 38861489 |
| 10764 | 27: 39015604 | 10764 | 27: 39015763 | 10764 | 27: 39015976 | 10766 | 27: 39667527 | 10766 | 27: 39668431 |
| 10767 | 27: 39780803 | 10768 | 27: 40004933 | 10769 | 27: 40356156 | 10769 | 27: 40357028 | 10769 | 27: 40414746 |
| 10770 | 27: 40674026 | 10770 | 27: 40868181 | 10771 | 27: 41044067 | 10772 | 27: 41178088 | 10773 | 27: 41583577 |
| 10774 | 27: 41702146 | 10775 | 27: 41886684 | 10776 | 27: 42166403 | 10776 | 27: 42228982 | 10776 | 27: 42242635 |
| 10777 | 27: 42427599 | 10777 | 27: 42462522 | 10777 | 27: 42487443 | 10778 | 27: 42794556 | 10781 | 27: 43428164 |
| 10783 | 27: 43823427 | 10783 | 27: 43863511 | 10783 | 27: 43908553 | 10784 | 27: 43927325 | 10784 | 27: 43987089 |
| 10785 | 27: 43995458 | 10785 | 27: 44200677 | 10787 | 27: 44375041 | 10787 | 27: 44412495 | 10788 | 27: 44543531 |
| 10788 | 27: 44629778 | 10792 | 28: 479125 | 10792 | 28: 504407 | 10793 | 28: 692680 | 10793 | 28: 704706 |
| 10793 | 28: 704958 | 10794 | 28: 1196781 | 10795 | 28: 1230989 | 10795 | 28: 1234367 | 10795 | 28: 1243626 |
| 10795 | 28: 1243641 | 10795 | 28: 1243692 | 10795 | 28: 1320241 | 10796 | 28: 1399889 | 10796 | 28: 1487331 |
| 10796 | 28: 1489641 | 10798 | 28: 1783556 | 10798 | 28: 1818831 | 10798 | 28: 1840156 | 10799 | 28: 1924353 |
| 10799 | 28: 1964866 | 10799 | 28: 2032083 | 10800 | 28: 2144906 | 10800 | 28: 2162175 | 10801 | 28: 2343692 |
| 10802 | 28: 2709799 | 10802 | 28: 2730701 | 10803 | 28: 3272910 | 10803 | 28: 3476063 | 10803 | 28: 3548826 |
| 10803 | 28: 3590983 | 10804 | 28: 3973141 | 10804 | 28: 3973211 | 10804 | 28: 3984834 | 10805 | 28: 4431180 |
| 10805 | 28: 4631548 | 10806 | 28: 4701678 | 10806 | 28: 4907755 | 10807 | 28: 5014037 | 10808 | 28: 5220057 |
| 10808 | 28: 5220146 | 10808 | 28: 5220186 | 10808 | 28: 5245742 | 10808 | 28: 5247877 | 10808 | 28: 5253360 |
| 1081C | 28: 5630191 | 10810 | 28: 5815635 | 10810 | 28: 5833150 | 10811 | 28: 5839078 | 10811 | 28: 5870781 |
| 10811 | 28: 5870881 | 10811 | 28: 5873548 | 10811 | 28: 5873612 | 10811 | 28: 5873655 | 10811 | 28: 5874287 |
| 10811 | 28: 5903348 | 10814 | 28: 6691792 | 10815 | 28: 6866814 | 10815 | 28: 6866847 | 10815 | 28: 6866914 |
| 10816 | 28: 7257360 | 10817 | 28: 7304728 | 10817 | 28: 7321818 | 10817 | 28: 7339237 | 10817 | 28: 7378556 |
| 10817 | 28: 7415736 | 10818 | 28: 7555026 | 10818 | 28: 7760692 | 10819 | 28: 7786515 | 10819 | 28: 7813529 |
| 10820 | 28: 8105292 | 10820 | 28: 8125888 | 10821 | 28: 8201519 | 10821 | 28: 8331969 | 10822 | 28: 8547431 |
| 10823 | 28: 8650652 | 10823 | 28: 8698842 | 10823 | 28: 8707863 | 10824 | 28: 8843955 | 10824 | 28: 9025349 |
| 10825 | 28: 9100351 | 10825 | 28: 9269631 | 10826 | 28: 9336102 | 10826 | 28: 9386021 | 10826 | 28: 9586664 |
| 10828 | 28: 9807554 | 10828 | 28: 9913957 | 10828 | 28: 9914017 | 10828 | 28: 9915219 | 10828 | 28: 9915233 |
| 10830 | 28: 10277520 | 10835 | 28: 12225835 | 10837 | 28: 12371401 | 10838 | 28: 12623784 | 10838 | 28: 12623831 |
| 10838 | 28: 12623855 | 10840 | 28: 13022825 | 10841 | 28: 13252341 | 10843 | 28: 13950890 | 10844 | 28: 14104824 |
| 10844 | 28: 14144978 | 10844 | 28: 14157633 | 10844 | 28: 14181805 | 10844 | 28: 14204540 | 10845 | 28: 14219987 |
| 10845 | 28: 14342309 | 10845 | 28: 14415424 | 10845 | 28: 14415472 | 10846 | 28: 14547089 | 10846 | 28: 14552705 |
| 10847 | 28: 14925580 | 10848 | 28: 14960497 | 10848 | 28: 15003137 | 10849 | 28: 15386201 | 10849 | 28: 15392967 |
| 10850 | 28: 15541195 | 10850 | 28: 15543708 | 10850 | 28: 15562551 | 10851 | 28: 15866947 | 10851 | 28: 15875883 |
| 10851 | 28: 15913989 | 10851 | 28: 15913997 | 10852 | 28: 15914061 | 10852 | 28: 15914091 | 10852 | 28: 15942493 |
| 10852 | 28: 15972055 | 10852 | 28: 16010478 | 10853 | 28: 16039905 | 10853 | 28: 16124447 | 10853 | 28: 16124496 |
| 10854 | 28: 16335973 | 10854 | 28: 16387453 | 10854 | 28: 16407912 | 10855 | 28: 16520465 | 10855 | 28: 16588164 |
| 10855 | 28: 16644285 | 10855 | 28: 16644893 | 10855 | 28: 16648829 | 10856 | 28: 16661175 | 10856 | 28: 16661840 |
| 10856 | 28: 16692080 | 10856 | 28: 16767636 | 10856 | 28: 16773322 | 10856 | 28: 16818843 | 10857 | 28: 16930270 |
| 10858 | 28: 17215906 | 10858 | 28: 17282866 | 10859 | 28: 17285666 | 10859 | 28: 17285711 | 10860 | 28: 17588476 |
| 10861 | 28: 17835910 | 10862 | 28: 18019905 | 10862 | 28: 18019907 | 10862 | 28: 18019913 | 10862 | 28: 18019925 |
| 10863 | 28: 18086730 | 10863 | 28: 18107994 | 10864 | 28: 18485866 | 10866 | 28: 18937069 | 10866 | 28: 19041963 |
| 10866 | 28: 19127485 | 10867 | 28: 19229652 | 10868 | 28: 19534804 | 10868 | 28: 19583995 | 10868 | 28: 19673238 |
| 10869 | 28: 19774362 | 10871 | 28: 20415689 | 10875 | 28: 21574143 | 10878 | 28: 22399855 | 10878 | 28: 22479622 |
| 10879 | 28: 22620494 | 10879 | 28: 22620545 | 10879 | 28: 22620582 | 10880 | 28: 22847773 | 10880 | 28: 23007374 |
| 10880 | 28: 23007384 | 10880 | 28: 23049818 | 10881 | 28: 23173525 | 10881 | 28: 23177219 | 10882 | 28: 23292046 |
| 10882 | 28: 23296757 | 10882 | 28: 23385467 | 10883 | 28: 23385512 | 10885 | 28: 24001450 | 10886 | 28: 24160079 |
| 10886 | 28: 24160811 | 10886 | 28: 24242145 | 10886 | 28: 24253020 | 10888 | 28: 24623959 | 10889 | 28: 24802411 |
| 10889 | 28: 24802550 | 10889 | 28: 24802551 | 10889 | 28: 24885896 | 10890 | 28: 24934653 | 10890 | 28: 25064734 |
| 10890 | 28: 25090839 | 10890 | 28: 25139537 | 10891 | 28: 25385010 | 10891 | 28: 25408853 | 10892 | 28: 25415208 |
| 10892 | 28: 25614547 | 10892 | 28: 25625624 | 10893 | 28: 25625649 | 10893 | 28: 25625667 | 10893 | 28: 25626954 |
| 10893 | 28: 25731593 | 10893 | 28: 25808647 | 10894 | 28: 25947218 | 10894 | 28: 26030383 | 10894 | 28: 26036200 |
| 10895 | 28: 26155221 | 10895 | 28: 26180213 | 10896 | 28: 26181587 | 10896 | 28: 26181622 | 10896 | 28: 26181655 |
| 10896 | 28: 26185280 | 10896 | 28: 26185344 | 10896 | 28: 26185376 | 10896 | 28: 26186082 | 10896 | 28: 26186104 |
| 10896 | 28: 26186116 | 10896 | 28: 26186150 | 10898 | 28: 26504153 | 10898 | 28: 26606263 | 10899 | 28: 26681661 |
| 10899 | 28: 26740880 | 10899 | 28: 26752280 | 10899 | 28: 26814555 | 10900 | 28: 27066599 | 10900 | 28: 27068380 |
| 10901 | 28: 27070779 | 10901 | 28: 27146373 | 10902 | 28: 27317961 | 10903 | 28: 27518059 | 10903 | 28: 27566739 |
| 10904 | 28: 27704605 | 10904 | 28: 27851635 | 10905 | 28: 27937188 | 10905 | 28: 27937338 | 10905 | 28: 27937971 |
| 10906 | 28: 27941915 | 10906 | 28: 27941916 | 10906 | 28: 27941932 | 10906 | 28: 27943776 | 10906 | 28: 27943868 |
| 10906 | 28: 27943901 | 10906 | 28: 27943916 | 10906 | 28: 27948642 | 10906 | 28: 27948643 | 10907 | 28: 27954047 |
| 10907 | 28: 28109673 | 10908 | 28: 28192038 | 10908 | 28: 28290243 | 10909 | 28: 28290255 | 10909 | 28: 28290934 |
| 10909 | 28: 28290955 | 10909 | 28: 28290999 | 10909 | 28: 28313393 | 10910 | 28: 28407860 | 10910 | 28: 28646964 |
| 10910 | 28: 28722550 | 10911 | 28: 28920066 | 10911 | 28: 28920811 | 10911 | 28: 28920813 | 10913 | 28: 29172597 |
| 10913 | 28: 29210804 | 10913 | 28: 29213687 | 10913 | 28: 29236992 | 10913 | 28: 29298781 | 10915 | 28: 29704456 |
| 10915 | 28: 29735862 | 10915 | 28: 29737399 | 10915 | 28: 29776190 | 10916 | 28: 29777653 | 10916 | 28: 29841383 |
| 10916 | 28: 29914673 | 10917 | 28: 30046702 | 10917 | 28: 30226392 | 10918 | 28: 30648480 | 10919 | 28: 30730091 |
| 10919 | 28: 30770433 | 10919 | 28: 30824174 | 10920 | 28: 30869446 | 10920 | 28: 30999414 | 10920 | 28: 31017300 |
| 10920 | 28: 31042068 | 10921 | 28: 31093235 | 10921 | 28: 31386148 | 10922 | 28: 31501902 | 10922 | 28: 31528266 |
| 10922 | 28: 31531949 | 10923 | 28: 31783641 | 10925 | 28: 32102216 | 10925 | 28: 32102950 | 10925 | 28: 32104334 |
| 10925 | 28: 32119157 | 10925 | 28: 32119371 | 10925 | 28: 32122100 | 10925 | 28: 32141403 | 10926 | 28: 32322725 |
| 10927 | 28: 32385967 | 10927 | 28: 32392342 | 10930 | 28: 32904173 | 10930 | 28: 32960277 | 10931 | 28: 32997331 |
| 10931 | 28: 33041333 | 10932 | 28: 33158395 | 10932 | 28: 33186798 | 10932 | 28: 33225867 | 10932 | 28: 33239306 |
| 10933 | 28: 33329500 | 10933 | 28: 33342708 | 10933 | 28: 33368759 | 10934 | 28: 33551708 | 10934 | 28: 33555527 |
| 10934 | 28: 33662006 | 10935 | 28: 33744728 | 10935 | 28: 33822791 | 10935 | 28: 33823084 | 10936 | 28: 34010065 |
| 10936 | 28: 34062194 | 10937 | 28: 34243304 | 10937 | 28: 34254734 | 10937 | 28: 34333090 | 10938 | 28: 34496870 |
| 10938 | 28: 34523399 | 10939 | 28: 34603788 | 10940 | 28: 34778574 | 10940 | 28: 34786048 | 10940 | 28: 34828202 |
| 10941 | 28: 34853555 | 10941 | 28: 34912784 | 10941 | 28: 34993324 | 10942 | 28: 35011293 | 10942 | 28: 35125823 |
| 10942 | 28: 35132199 | 10942 | 28: 35132247 | 10943 | 28: 35222450 | 10943 | 28: 35291319 | 10944 | 28: 35644657 |
| 10944 | 28: 35671648 | 10944 | 28: 35672175 | 10944 | 28: 35680732 | 10944 | 28: 35683234 | 10944 | 28: 35685429 |
| 10945 | 28: 35805891 | 10945 | 28: 35837667 | 10945 | 28: 35923596 | 10946 | 28: 36015548 | 10947 | 28: 36205983 |
| 10948 | 28: 36587691 | 10952 | 28: 37533725 | 10954 | 28: 37721207 | 10960 | 28: 39163650 | 10960 | 28: 39164865 |
| 10960 | 28: 39164902 | 10960 | 28: 39164949 | 10960 | 28: 39167717 | 10960 | 28: 39167797 | 10960 | 28: 39167807 |
| 10960 | 28: 39167808 | 10960 | 28: 39167836 | 10960 | 28: 39167853 | 10961 | 28: 39266326 | 10961 | 28: 39290663 |
| 10961 | 28: 39316523 | 10963 | 28: 39818069 | 10963 | 28: 39868243 | 10966 | 28: 40526877 | 10966 | 28: 40526886 |
| 10966 | 28: 40547129 | 10966 | 28: 40606521 | 10966 | 28: 40609071 | 10967 | 28: 40613418 | 10967 | 28: 40641661 |
| 10967 | 28: 40644608 | 10967 | 28: 40647540 | 10967 | 28: 40648502 | 10967 | 28: 40649183 | 10967 | 28: 40650128 |
| 10967 | 28: 40650866 | 10968 | 28: 40651548 | 10968 | 28: 40652228 | 10968 | 28: 40653623 | 10968 | 28: 40654155 |
| 10968 | 28: 40654919 | 10968 | 28: 40655651 | 10968 | 28: 40656367 | 10968 | 28: 40658031 | 10968 | 28: 40659095 |
| 10969 | 28: 40679353 | 10969 | 28: 40683789 | 10969 | 28: 40684338 | 10969 | 28: 40685176 | 10969 | 28: 40685911 |
| 10969 | 28: 40687468 | 10969 | 28: 40688010 | 10969 | 28: 40688917 | 10969 | 28: 40690412 | 10969 | 28: 40691166 |
| 10970 | 28: 40692798 | 10970 | 28: 40693384 | 10970 | 28: 40694350 | 10970 | 28: 40695394 | 10970 | 28: 40697087 |
| 10970 | 28: 40698875 | 10970 | 28: 40700189 | 10970 | 28: 40701565 | 10970 | 28: 40707053 | 10970 | 28: 40707560 |
| 10971 | 28: 40708330 | 10971 | 28: 40709943 | 10971 | 28: 40711617 | 10971 | 28: 40714408 | 10971 | 28: 40716186 |
| 10971 | 28: 40717895 | 10971 | 28: 40718746 | 10971 | 28: 40719687 | 10971 | 28: 40720636 | 10971 | 28: 40721303 |
| 10972 | 28: 40722936 | 10972 | 28: 40723874 | 10972 | 28: 40724503 | 10972 | 28: 40725696 | 10972 | 28: 40726977 |
| 10973 | 28: 40857491 | 10974 | 28: 40977297 | 10974 | 28: 41002895 | 10974 | 28: 41077744 | 10975 | 28: 41244211 |
| 10975 | 28: 41283427 | 10975 | 28: 41333995 | 10977 | 28: 41565674 | 10978 | 28: 41666097 | 10978 | 28: 41666186 |
| 10978 | 28: 41724320 | 10978 | 28: 41728249 | 10978 | 28: 41731568 | 10978 | 28: 41757290 | 10979 | 28: 41885934 |
| 10979 | 28: 41913803 | 10980 | 28: 41952391 | 10980 | 28: 41980728 | 10980 | 28: 41986845 | 10980 | 28: 41989663 |
| 10980 | 28: 42038658 | 10981 | 28: 42092356 | 10982 | 28: 42424892 | 10983 | 28: 42588414 | 10983 | 28: 42648031 |
| 10984 | 28: 42816073 | 10984 | 28: 42822333 | 10984 | 28: 42894677 | 10985 | 28: 43061595 | 10985 | 28: 43097491 |
| 10985 | 28: 43111417 | 10985 | 28: 43111489 | 10985 | 28: 43111570 | 10985 | 28: 43112699 | 10986 | 28: 43112752 |
| 10986 | 28: 43112807 | 10986 | 28: 43112817 | 10986 | 28: 43112858 | 10986 | 28: 43140781 | 10987 | 28: 43230125 |
| 10987 | 28: 43258213 | 10988 | 28: 43420442 | 10988 | 28: 43482124 | 10988 | 28: 43501640 | 10988 | 28: 43538406 |
| 10989 | 28: 43579200 | 10989 | 28: 43651140 | 10990 | 28: 44028372 | 10990 | 28: 44030407 | 10990 | 28: 44031081 |
| 10990 | 28: 44038721 | 10991 | 28: 44039225 | 10991 | 28: 44039304 | 10991 | 28: 44143344 | 10991 | 28: 44157666 |
| 10992 | 28: 44264765 | 10993 | 28: 44453518 | 10994 | 28: 44687207 | 10994 | 28: 44852965 | 10996 | 28: 45172059 |
| 10997 | 28: 45281713 | 10997 | 28: 45314101 | 10998 | 28: 45416826 | 10998 | 28: 45476974 | 10999 | 28: 45579104 |
| 11000 | 28: 45713679 | 11000 | 28: 45771532 | 11000 | 28: 45847568 | 11001 | 29: 612126 | 11001 | 29: 699249 |
| 11002 | 29: 699337 | 11002 | 29: 699732 | 11002 | 29: 699767 | 11002 | 29: 750365 | 11004 | 29: 1219671 |
| 11004 | 29: 1273559 | 11004 | 29: 1315459 | 11005 | 29: 1429261 | 11005 | 29: 1499682 | 11005 | 29: 1512567 |
| 11006 | 29: 1512626 | 11007 | 29: 1772440 | 11008 | 29: 2136150 | 11009 | 29: 2325537 | 11009 | 29: 2380491 |
| 11011 | 29: 2594653 | 11011 | 29: 2620380 | 11013 | 29: 2994930 | 11014 | 29: 3175555 | 11015 | 29: 3406656 |
| 11016 | 29: 3717232 | 11019 | 29: 4246237 | 11019 | 29: 4406319 | 11020 | 29: 4643160 | 11022 | 29: 5107639 |
| 11025 | 29: 6283155 | 11026 | 29: 6283208 | 11027 | 29: 6779650 | 11028 | 29: 7101326 | 11029 | 29: 7255100 |
| 11029 | 29: 7426428 | 11033 | 29: 8601758 | 11034 | 29: 8772433 | 11035 | 29: 8793187 | 11035 | 29: 8821458 |
| 11035 | 29: 8883493 | 11035 | 29: 8926820 | 11036 | 29: 8998775 | 11036 | 29: 9022834 | 11036 | 29: 9022891 |
| 11036 | 29: 9102521 | 11037 | 29: 9365821 | 11038 | 29: 9490114 | 11040 | 29: 9846120 | 11040 | 29: 9882247 |
| 11042 | 29: 10340072 | 11044 | 29: 10979545 | 11046 | 29: 11204386 | 11046 | 29: 11241638 | 11046 | 29: 11260132 |
| 11046 | 29: 11341144 | 11047 | 29: 11709562 | 11048 | 29: 11839933 | 11048 | 29: 11891707 | 11049 | 29: 11952953 |
| 11049 | 29: 11977550 | 11049 | 29: 12018235 | 11049 | 29: 12084416 | 11050 | 29: 12114717 | 11050 | 29: 12138482 |
| 11050 | 29: 12182249 | 11051 | 29: 12394797 | 11051 | 29: 12538136 | 11051 | 29: 12589589 | 11052 | 29: 12619588 |
| 11052 | 29: 12622700 | 11054 | 29: 12987411 | 11055 | 29: 13574420 | 11055 | 29: 13574513 | 11056 | 29: 13821500 |
| 11058 | 29: 14373492 | 11058 | 29: 14374640 | 11059 | 29: 14741850 | 11059 | 29: 14741861 | 11059 | 29: 14741873 |
| 11059 | 29: 14741887 | 11060 | 29: 14741921 | 11060 | 29: 14900262 | 11061 | 29: 15134872 | 11062 | 29: 15304699 |
| 11062 | 29: 15395863 | 11064 | 29: 15811607 | 11066 | 29: 16464456 | 11066 | 29: 16485296 | 11069 | 29: 16964102 |
| 11069 | 29: 16992973 | 11069 | 29: 17055985 | 11070 | 29: 17207473 | 11070 | 29: 17442223 | 11071 | 29: 17505934 |
| 11071 | 29: 17540650 | 11071 | 29: 17700379 | 11073 | 29: 18393448 | 11073 | 29: 18612768 | 11078 | 29: 20127489 |
| 11079 | 29: 20506894 | 11079 | 29: 20644568 | 11080 | 29: 20651157 | 11080 | 29: 20673987 | 11080 | 29: 20674048 |
| 11082 | 29: 21228889 | 11082 | 29: 21315170 | 11083 | 29: 21392121 | 11083 | 29: 21392184 | 11083 | 29: 21392189 |
| 11084 | 29: 21600071 | 11086 | 29: 22072294 | 11086 | 29: 22180877 | 11086 | 29: 22297264 | 11086 | 29: 22344163 |
| 11088 | 29: 22760861 | 11088 | 29: 22836272 | 11090 | 29: 23318559 | 11090 | 29: 23357907 | 11091 | 29: 23560620 |
| 11092 | 29: 23583982 | 11092 | 29: 23628287 | 11092 | 29: 23661992 | 11092 | 29: 23731784 | 11093 | 29: 23850587 |
| 11093 | 29: 23937388 | 11094 | 29: 24158412 | 11095 | 29: 24258248 | 11095 | 29: 24330084 | 11095 | 29: 24354233 |
| 11095 | 29: 24362811 | 11095 | 29: 24490616 | 11096 | 29: 24840874 | 11097 | 29: 25015460 | 11098 | 29: 25071999 |
| 11098 | 29: 25205531 | 11099 | 29: 25222326 | 11099 | 29: 25305438 | 11099 | 29: 25371765 | 11099 | 29: 25438251 |
| 11101 | 29: 25903150 | 11101 | 29: 25908734 | 11101 | 29: 26070069 | 11102 | 29: 26105651 | 11103 | 29: 26549594 |
| 11104 | 29: 26946250 | 11104 | 29: 26946336 | 11104 | 29: 26946383 | 11105 | 29: 26947201 | 11105 | 29: 26947224 |
| 11105 | 29: 26962161 | 11106 | 29: 27537716 | 11106 | 29: 27537760 | 11106 | 29: 27538148 | 11107 | 29: 27595444 |
| 11107 | 29: 27728132 | 11108 | 29: 27790794 | 11108 | 29: 27808076 | 11108 | 29: 27808486 | 11108 | 29: 27823998 |
| 11108 | 29: 27824016 | 11108 | 29: 27824039 | 11109 | 29: 27824126 | 11109 | 29: 28026650 | 11109 | 29: 28100144 |
| 11110 | 29: 28218734 | 11110 | 29: 28226254 | 11110 | 29: 28228229 | 11110 | 29: 28251283 | 111111 | 29: 28278699 |
| 111117 | 29: 28370448 | 111118 | 29: 28430575 | 11112 | 29: 28519839 | 11112 | 29: 28574787 | 11112 | 29: 28655873 |
| 11112 | 29: 28715263 | 11113 | 29: 28815692 | 11113 | 29: 28826292 | 11115 | 29: 29397513 | 11116 | 29: 29397515 |
| 11116 | 29: 29461701 | 11116 | 29: 29472010 | 11116 | 29: 29555287 | 11117 | 29: 29629619 | 11117 | 29: 29649318 |
| 11117 | 29: 29653990 | 11117 | 29: 29665600 | 11117 | 29: 29676297 | 11117 | 29: 29682536 | 11117 | 29: 29690811 |
| 11118 | 29: 29755215 | 11118 | 29: 29755216 | 11118 | 29: 29755261 | 11118 | 29: 29755320 | 11118 | 29: 29756679 |
| 11118 | 29: 29756692 | 11119 | 29: 29928478 | 11119 | 29: 29928524 | 11119 | 29: 29928553 | 11119 | 29: 29928591 |
| 11120 | 29: 29928636 | 11120 | 29: 29928675 | 11120 | 29: 29928677 | 11121 | 29: 30201851 | 11121 | 29: 30294652 |
| 11123 | 29: 30759911 | 11124 | 29: 30759984 | 11125 | 29: 31124828 | 11126 | 29: 31355454 | 11126 | 29: 31355522 |
| 11127 | 29: 31455230 | 11127 | 29: 31488540 | 11128 | 29: 31676141 | 11128 | 29: 31801460 | 11128 | 29: 31801542 |
| 11128 | 29: 31845002 | 11128 | 29: 31845960 | 11130 | 29: 32253717 | 11131 | 29: 32397724 | 11131 | 29: 32544942 |
| 11131 | 29: 32611173 | 11132 | 29: 32693113 | 11132 | 29: 32753972 | 11132 | 29: 32754754 | 11132 | 29: 32764982 |
| 11132 | 29: 32765663 | 11132 | 29: 32766346 | 11133 | 29: 32842956 | 11134 | 29: 33120665 | 11135 | 29: 33560766 |
| 11136 | 29: 33704537 | 11137 | 29: 33824948 | 11139 | 29: 34433856 | 11141 | 29: 34688201 | 11142 | 29: 34751704 |
| 11142 | 29: 34751748 | 11143 | 29: 34985663 | 11144 | 29: 35373548 | 11144 | 29: 35393862 | 11144 | 29: 35393931 |
| 11145 | 29: 35506238 | 11146 | 29: 35663595 | 11146 | 29: 35697578 | 11147 | 29: 35864204 | 11147 | 29: 35918743 |
| 11148 | 29: 36106383 | 11148 | 29: 36108259 | 11149 | 29: 36148048 | 11149 | 29: 36206784 | 11149 | 29: 36207683 |
| 11150 | 29: 36388797 | 11151 | 29: 36481048 | 11152 | 29: 36766544 | 11152 | 29: 36775708 | 11152 | 29: 36800395 |
| 11152 | 29: 36811950 | 11152 | 29: 36813596 | 11153 | 29: 36934958 | 11153 | 29: 36975121 | 11154 | 29: 37168472 |
| 11154 | 29: 37221849 | 11155 | 29: 37332886 | 11155 | 29: 37332893 | 11155 | 29: 37342276 | 11155 | 29: 37411101 |
| 11156 | 29: 37440660 | 11156 | 29: 37458594 | 11156 | 29: 37523548 | 11160 | 29: 39536492 | 11161 | 29: 39891282 |
| 11162 | 29: 39975025 | 11163 | 29: 40151594 | 11163 | 29: 40297458 | 11163 | 29: 40314843 | 11164 | 29: 40481400 |
| 11164 | 29: 40518208 | 11164 | 29: 40595649 | 11165 | 29: 40805372 | 11165 | 29: 40806225 | 11166 | 29: 40851566 |
| 11167 | 29: 40878959 | 11167 | 29: 40879481 | 11167 | 29: 40919327 | 11167 | 29: 41004847 | 11168 | 29: 41038278 |
| 11168 | 29: 41142315 | 11168 | 29: 41142793 | 11169 | 29: 41279012 | 11169 | 29: 41338346 | 11169 | 29: 41342454 |
| 11170 | 29: 41493354 | 11171 | 29: 41557548 | 11171 | 29: 41564691 | 11171 | 29: 41642963 | 11171 | 29: 41687163 |
| 11171 | 29: 41749646 | 11172 | 29: 41785049 | 11172 | 29: 41789280 | 11172 | 29: 41792341 | 11172 | 29: 41823721 |
| 11172 | 29: 41947165 | 11173 | 29: 41976753 | 11173 | 29: 42018005 | 11173 | 29: 42022273 | 11173 | 29: 42031453 |
| 11175 | 29: 42391692 | 11175 | 29: 42509147 | 11175 | 29: 42513501 | 11176 | 29: 42528204 | 11176 | 29: 42615699 |
| 11176 | 29: 42635388 | 11176 | 29: 42642354 | 11176 | 29: 42657038 | 11176 | 29: 42659255 | 11176 | 29: 42659448 |
| 11177 | 29: 42844516 | 11178 | 29: 42982094 | 11178 | 29: 43052218 | 11178 | 29: 43090186 | 11179 | 29: 43206957 |
| 11179 | 29: 43207132 | 11179 | 29: 43207525 | 11179 | 29: 43207573 | 11179 | 29: 43207753 | 11179 | 29: 43210991 |
| 11179 | 29: 43218240 | 11180 | 29: 43282578 | 11180 | 29: 43391770 | 11181 | 29: 43495617 | 11181 | 29: 43524321 |
| 11182 | 29: 43620118 | 11182 | 29: 43622532 | 11182 | 29: 43627877 | 11182 | 29: 43631768 | 11183 | 29: 43704875 |
| 11183 | 29: 43750816 | 11183 | 29: 43750820 | 11183 | 29: 43814690 | 11183 | 29: 43827364 | 11183 | 29: 43880340 |
| 11184 | 29: 43883371 | 11184 | 29: 43909067 | 11184 | 29: 43968492 | 11184 | 29: 43970000 | 11184 | 29: 43981948 |
| 11184 | 29: 43982210 | 11186 | 29: 44265806 | 11186 | 29: 44285086 | 11186 | 29: 44314568 | 11187 | 29: 44423551 |
| 11188 | 29: 44484826 | 11188 | 29: 44552650 | 11188 | 29: 44627679 | 11189 | 29: 44770084 | 11189 | 29: 44991893 |
| 11190 | 29: 45224759 | 11191 | 29: 45378882 | 11191 | 29: 45460407 | 11191 | 29: 45538531 | 11192 | 29: 45566486 |
| 11192 | 29: 45574701 | 11192 | 29: 45601782 | 11192 | 29: 45749221 | 11193 | 29: 45756539 | 11193 | 29: 45875877 |
| 11193 | 29: 45897665 | 11193 | 29: 45951010 | 11194 | 29: 45996529 | 11194 | 29: 46069944 | 11195 | 29: 46268948 |
| 11195 | 29: 46344185 | 11196 | 29: 46344338 | 11199 | 29: 47058184 | 11199 | 29: 47089599 | 11199 | 29: 47171895 |
| 11200 | 29: 47412530 | 11200 | 29: 47512321 | 11201 | 29: 47628422 | 11203 | 29: 47965964 | 11203 | 29: 48004279 |
| 11204 | 29: 48072601 | 11205 | 29: 48380729 | 11205 | 29: 48412845 | 11205 | 29: 48499387 | 11206 | 29: 48750418 |
| 11207 | 29: 48886024 | 11207 | 29: 48936775 | 11210 | 29: 49277470 | 11211 | 29: 49503212 | 11211 | 29: 49583754 |
| 11211 | 29: 49601539 | 11211 | 29: 49645960 | 11211 | 29: 49646022 | 11212 | 29: 49706299 | 11213 | 29: 50015783 |
| 11214 | 29: 50279949 | 11214 | 29: 50279990 | 11214 | 29: 50326170 | 11215 | 29: 50499209 | 11215 | 29: 50566734 |
| 11215 | 29: 50572585 | 11215 | 29: 50573514 | 11215 | 29: 50595441 | 11216 | 29: 50698048 | 11216 | 29: 50698051 |
| 11216 | 29: 50698125 | 11216 | 29: 50699876 | 11216 | 29: 50700624 | 11217 | 29: 50702620 | 11217 | 29: 50777521 |
| 11217 | 29: 50809985 | 11217 | 29: 50901915 | 11217 | 29: 50902670; | | | | |

and
the sites of the third marker panel are shown in the following table:

| NO. | Position | NO. | Position | NO. | Position | NO. | Position | NO. | Position |
|---|---|---|---|---|---|---|---|---|---|
| 188 | 1:395886 | 339 | 1:703722 | 420 | 1:906974 | 1171 | 1:2996340 | 1536 | 1:4057672 |
| 2248 | 1:589653 | 2403 | 1:626739 | 3852 | 1:985197 | 4392 | 1:1131760 | 4969 | 1:1264288 |
| 6050 | 1:149646 | 6468 | 1:156749 | 6762 | 2:537453 | 6787 | 2:5824469 | 6980 | 2:1042500 |
| 7049 | 2:128028 | 7090 | 2:138220 | 7681 | 2:269491 | 8255 | 2:4182328 | 8310 | 2:4316452 |
| 8401 | 2:457305 | 1090 | 2:110396 | 1119 | 2:117962 | 1166 | 2:1282694 | 1277 | 3:1267167 |
| 1313 | 3:211447 | 1403 | 3:395491 | 1406 | 3:402555 | 1410 | 3:4160522 | 1442 | 3:4954803 |
| 1453 | 3:518176 | 1475 | 3:578965 | 1529 | 3:734325 | 1573 | 3:8499077 | 1615 | 3:9391427 |
| 1628 | 3:975958 | 1723 | 3:115822 | 1798 | 4:109063 | 1818 | 4:1621515 | 1838 | 4:2065221 |
| 1897 | 4:367420 | 1915 | 4:414001 | 1971 | 4:574214 | 2019 | 4:7061032 | 2025 | 4:7253292 |
| 2047 | 4:779178 | 2197 | 4:109990 | 2288 | 5:765657 | 2371 | 5:2767099 | 2388 | 5:3169176 |
| 2505 | 5:629338 | 2520 | 5:666939 | 2543 | 5:719145 | 2568 | 5:7804644 | 2622 | 5:8792394 |
| 2671 | 5:976312 | 2750 | 5:112616 | 2783 | 5:118152 | 2851 | 6:1273217 | 2895 | 6:2221084 |
| 2919 | 6:281131 | 2919 | 6:281318 | 3226 | 6:429944 | 3250 | 6:4536462 | 3309 | 6:5955160 |
| 3311 | 6:598689 | 3355 | 6:728525 | 3507 | 6:109291 | 3527 | 6:1134849 | 3591 | 7:7248738 |
| 3605 | 7:121749 | 3634 | 7:172230 | 3671 | 7:248614 | 3790 | 7:5323337 | 3903 | 7:7930430 |
| 3904 | 7:793814 | 3910 | 7:805361 | 3961 | 7:918583 | 3970 | 7:9452918 | 4012 | 7:1009550 |
| 4031 | 7:105797 | 4055 | 8:168853 | 4167 | 8:287478 | 4284 | 8:5958533 | 4394 | 8:8752304 |
| 4449 | 8:101431 | 4466 | 8:104418 | 4553 | 9:142246 | 4608 | 9:2777847 | 4704 | 9:5170207 |
| 4729 | 9:585114 | 4832 | 9:822086 | 4908 | 9:970297 | 4966 | 10:358243 | 5021 | 10:146014 |
| 5033 | 10:16803 | 5143 | 10:44059 | 5146 | 10:44872 | 5192 | 10:555395 | 5212 | 10:614624 |
| 5294 | 10:81223 | 5300 | 10:82399 | 5307 | 10:84068 | 5367 | 10:971526 | 5448 | 11:940047 |
| 5509 | 11:24487 | 5558 | 11:36861 | 5596 | 11:46544 | 5660 | 11:620426 | 5663 | 11:627814 |
| 5678 | 11:66365 | 5735 | 11:77817 | 5818 | 11:95543 | 5851 | 11:102567 | 5855 | 11:103007 |
| 5918 | 12:11793 | 5977 | 12:25646 | 5999 | 12:31268 | 6090 | 12:578674 | 6099 | 12:609977 |
| 6124 | 12:68116 | 6155 | 12:75672 | 6156 | 12:76081 | 6217 | 13:207965 | 6317 | 13:253355 |
| 6422 | 13:47037 | 6513 | 13:68527 | 6542 | 13:74664 | 5584 | 13:826673 | 6638 | 14:911668 |
| 6643 | 14:10036 | 6757 | 14:26068 | 6840 | 14:46226 | 6866 | 14:534793 | 6896 | 14:602989 |
| 6969 | 14:77723 | 6977 | 14:79615 | 7089 | 15:20899 | 7148 | 15:329126 | 7170 | 15:374722 |
| 7170 | 15:37547 | 7194 | 15:42887 | 7229 | 15:49801 | 7231 | 15:506934 | 7297 | 15:638330 |
| 7359 | 15:77950 | 7425 | 16:92596 | 7433 | 16:11622 | 7440 | 16:130724 | 7460 | 16:191974 |
| 7675 | 16:71035 | 7696 | 16:75329 | 7727 | 17:10232 | 7766 | 17:913211 | 7781 | 17:127266 |
| 7801 | 17:17314 | 7846 | 17:29524 | 7847 | 17:29949 | 7853 | 17:314780 | 7929 | 17:504856 |
| 7946 | 17:54264 | 7998 | 17:64965 | 8050 | 18:18019 | 8074 | 18:674782 | 8150 | 18:233514 |
| 8157 | 18:24699 | 8166 | 18:26757 | 8251 | 18:46459 | 8268 | 18:485458 | 8283 | 18:521782 |
| 8345 | 18:64214 | 8371 | 19:44783 | 8389 | 19:82749 | 8403 | 19:108612 | 8423 | 19:150178 |
| 8432 | 19:16900 | 8553 | 19:38864 | 8558 | 19:40043 | 8579 | 19:441735 | 8624 | 19:522932 |
| 8636 | 19:54554 | 8673 | 19:59907 | 8708 | 20:23863 | 8795 | 20:178473 | 8854 | 20:309114 |
| 8917 | 20:46043 | 8962 | 20:58379 | 8983 | 20:63586 | 9034 | 21:304067 | 9114 | 21:208094 |
| 9134 | 21:26120 | 9139 | 21:27418 | 9192 | 21:39994 | 9205 | 21:431339 | 9241 | 21:516923 |
| 9288 | 21:60444 | 9301 | 21:63550 | 9383 | 22:11000 | 9386 | 22:117189 | 9424 | 22:209163 |
| 9428 | 22:21678 | 9431 | 22:22468 | 9440 | 22:25488 | 9546 | 22:505892 | 9559 | 22:534773 |
| 9573 | 22:55883 | 9635 | 23:73186 | 9740 | 23:27495 | 9815 | 23:409936 | 9820 | 23:420218 |
| 9868 | 23:51029 | 9883 | 24:16171 | 9903 | 24:61255 | 9928 | 24:104971 | 9946 | 24:151419 |
| 9970 | 24:21378 | 1006 | 24:44195 | 1011 | 24:55950 | 1017 | 25:313056 | 1023 | 25:145932 |
| 1025 | 25:19902 | 1028 | 25:27084 | 1028 | 25:27654 | 1040 | 26:819240 | 1042 | 26:131947 |
| 1052 | 26:37900 | 1053 | 26:39609 | 1057 | 26:49826 | 1062 | 27:920491 | 1063 | 27:117362 |
| 1064 | 27:13894 | 1065 | 27:16084 | 1067 | 27:19194 | 1068 | 27:224103 | 1074 | 27:357616 |
| 1075 | 27:37824 | 1077 | 27:41177 | 1081 | 28:58742 | 1085 | 28:159724 | 1087 | 28:203712 |
| 1093 | 28:33248 | 1094 | 28:35132 | 1099 | 28:43879 | 1103 | 29:910465 | 1104 | 29:111981 |
| 1111 | 29:28278 | 1111 | 29:28684 | 1118 | 29:44090 | 1121 | 29:508581; | | |

and
the sites of the fourth marker panel are shown in the following table:

| NO. | Position | NO. | Position | NO. | Position | NO. | Position | NO. | Position |
|---|---|---|---|---|---|---|---|---|---|
| 41 | 1: 1299095 | 42 | 1: 1301411 | 43 | 1: 1302242 | 72 | 1: 1675530 | 92 | 1: 2080108 |
| 105 | 1: 2256758 | 163 | 1: 3595729 | 167 | 1: 3620513 | 211 | 1: 4298693 | 229 | 1: 4729108 |
| 234 | 1: 4821451 | 287 | 1: 6049970 | 288 | 1: 6052470 | 289 | 1: 6054551 | 290 | 1: 6056604 |
| 291 | 1: 6061246 | 373 | 1: 7795006 | 388 | 1: 8249174 | 413 | 1: 8847132 | 417 | 1: 8982763 |
| 421 | 1: 9088695 | 423 | 1: 9118340 | 508 | 1: 10838284 | 509 | 1: 10876705 | 536 | 1: 11604750 |
| 538 | 1: 11752900 | 546 | 1: 12052406 | 553 | 1: 12362501 | 567 | 1: 12653822 | 629 | 1: 14114440 |
| 630 | 1: 14176114 | 721 | 1: 16635130 | 728 | 1: 16922627 | 747 | 1: 17462003 | 756 | 1: 17835385 |
| 763 | 1: 18110024 | 785 | 1: 18793633 | 806 | 1: 19543445 | 813 | 1: 19664427 | 930 | 1: 22893609 |
| 948 | 1: 23486726 | 949 | 1: 23493262 | 969 | 1: 24006741 | 984 | 1: 24500834 | 996 | 1: 24941722 |
| 1001 | 1: 25068708 | 1025 | 1: 25577980 | 1129 | 1: 29025677 | 1131 | 1: 29040742 | 1225 | 1: 31664947 |
| 1237 | 1: 32296642 | 1256 | 1: 32911987 | 1277 | 1: 33660199 | 1286 | 1: 33837966 | 1287 | 1: 33843390 |
| 1288 | 1: 33853447 | 1447 | 1: 37890316 | 1452 | 1: 38007855 | 1516 | 1: 39993737 | 1545 | 1: 40753557 |
| 1577 | 1: 41764542 | 1622 | 1: 42618603 | 1693 | 1: 44281769 | 1695 | 1: 44303304 | 1702 | 1: 44457020 |
| 1705 | 1: 44558674 | 1834 | 1: 47759114 | 1879 | 1: 49422581 | 1896 | 1: 49969737 | 2024 | 1: 53619617 |
| 2025 | 1: 53623176 | 2026 | 1: 53624697 | 2027 | 1: 53629301 | 2028 | 1: 53630113 | 2050 | 1: 54016006 |
| 2075 | 1: 54444238 | 2084 | 1: 54690665 | 2116 | 1: 55714510 | 2139 | 1: 56372440 | 2335 | 1: 61145631 |
| 2343 | 1: 61319735 | 2362 | 1: 61796684 | 2383 | 1: 62427513 | 2384 | 1: 62433558 | 2430 | 1: 63221728 |
| 2462 | 1: 64193131 | 2467 | 1: 64262857 | 2472 | 1: 64316563 | 2499 | 1: 64907342 | 2526 | 1: 65509479 |
| 2543 | 1: 66070553 | 2602 | 1: 67691264 | 2624 | 1: 68339701 | 2675 | 1: 69057373 | 2701 | 1: 69645645 |
| 2739 | 1: 70443699 | 2766 | 1: 70688109 | 2800 | 1: 71512292 | 2916 | 1: 74460838 | 2934 | 1: 74972802 |
| 2936 | 1: 74999820 | 2946 | 1: 75290882 | 2981 | 1: 76075161 | 3007 | 1: 76874012 | 3018 | 1: 77236634 |
| 3022 | 1: 77276581 | 3039 | 1: 77623057 | 3041 | 1: 77668859 | 3043 | 1: 77697633 | 3046 | 1: 77710839 |
| 3047 | 1: 77723763 | 3060 | 1: 78064459 | 3068 | 1: 78315800 | 3079 | 1: 78505641 | 3100 | 1: 79138567 |
| 3115 | 1: 79395243 | 3125 | 1: 79597067 | 3165 | 1: 80126772 | 3190 | 1: 80929167 | 3251 | 1: 82240750 |
| 3261 | 1: 82501216 | 3275 | 1: 82703570 | 3311 | 1: 83985563 | 3319 | 1: 84218035 | 3324 | 1: 84478357 |
| 3325 | 1: 84493668 | 3336 | 1: 85023039 | 3352 | 1: 85456698 | 3354 | 1: 85501954 | 3382 | 1: 86229426 |
| 3407 | 1: 86980692 | 3460 | 1: 88794416 | 3464 | 1: 88824366 | 3545 | 1: 90506530 | 3610 | 1: 92190821 |
| 3612 | 1: 92200875 | 3624 | 1: 92563287 | 3638 | 1: 93096031 | 3652 | 1: 93351663 | 3654 | 1: 93372952 |
| 3698 | 1: 94573894 | 3724 | 1: 95244942 | 3763 | 1: 96215196 | 3768 | 1: 96305965 | 3790 | 1: 96826926 |
| 3791 | 1: 96855345 | 3796 | 1: 96968145 | 3806 | 1: 97319365 | 3829 | 1: 97847508 | 3860 | 1: 98721777 |
| 3881 | 1: 99293036 | 3929 | 1: 100616517 | 3939 | 1: 100987282 | 4172 | 1: 108128123 | 4199 | 1: 108777437 |
| 4225 | 1: 109419276 | 4227 | 1: 109445956 | 4261 | 1: 110075319 | 4281 | 1: 110389266 | 4305 | 1: 111001754 |
| 4330 | 1: 111772744 | 4338 | 1: 111960165 | 4374 | 1: 112577975 | 4386 | 1: 112928362 | 4408 | 1: 113414092 |
| 4421 | 1: 113728857 | 4466 | 1: 114828912 | 4467 | 1: 114837165 | 4479 | 1: 115162087 | 4480 | 1: 115169020 |
| 4482 | 1: 115183751 | 4521 | 1: 116624187 | 4523 | 1: 116668988 | 4524 | 1: 116683980 | 4567 | 1: 117562616 |
| 4604 | 1: 118172592 | 4606 | 1: 118199028 | 4607 | 1: 118200955 | 4660 | 1: 119155291 | 4714 | 1: 120407128 |
| 4767 | 1: 121845822 | 4796 | 1: 122567824 | 4957 | 1: 126246695 | 4964 | 1: 126332119 | 5053 | 1: 128692434 |
| 5062 | 1: 128825902 | 5078 | 1: 129263274 | 5234 | 1: 132164555 | 5244 | 1: 132550146 | 5263 | 1: 133371805 |
| 5292 | 1: 134238188 | 5295 | 1: 134324146 | 5308 | 1: 134489385 | 5335 | 1: 135158239 | 5350 | 1: 135504827 |
| 5358 | 1: 135602604 | 5392 | 1: 135798830 | 5450 | 1: 137640097 | 5470 | 1: 138610452 | 5474 | 1: 138652323 |
| 5496 | 1: 139117138 | 5556 | 1: 140791857 | 5596 | 1: 141481667 | 5678 | 1: 142851690 | 5679 | 1: 142863118 |
| 5697 | 1: 143175205 | 5769 | 1: 144589518 | 5777 | 1: 144761021 | 5823 | 1: 145661981 | 5912 | 1: 147299699 |
| 5924 | 1: 147514403 | 5943 | 1: 147887268 | 5993 | 1: 148725405 | 6105 | 1: 150672403 | 6192 | 1: 152095555 |
| 6198 | 1: 152196690 | 6272 | 1: 153106216 | 6277 | 1: 153197360 | 6309 | 1: 153828547 | 6340 | 1: 154314444 |
| 6357 | 1: 154855892 | 6360 | 1: 154863975 | 6511 | 1: 157806548 | 6568 | 2: 1106317 | 6575 | 2: 1169877 |
| 6578 | 2: 1187683 | 6617 | 2: 1860169 | 6621 | 2: 1971521 | 6640 | 2: 2590948 | 6666 | 2: 3510323 |
| 6673 | 2: 3709938 | 6718 | 2: 4623152 | 6790 | 2: 5906646 | 6800 | 2: 6055644 | 6971 | 2: 10245713 |
| 6979 | 2: 10421191 | 7005 | 2: 11387282 | 7060 | 2: 13168802 | 7064 | 2: 13243953 | 7176 | 2: 16006718 |
| 7184 | 2: 16189620 | 7214 | 2: 16976649 | 7240 | 2: 17639779 | 7274 | 2: 18268568 | 7332 | 2: 19056221 |
| 7365 | 2: 19833213 | 7582 | 2: 24849703 | 7630 | 2: 25885564 | 7655 | 2: 26463668 | 7685 | 2: 27015449 |
| 7704 | 2: 27459813 | 7705 | 2: 27463924 | 7708 | 2: 27525591 | 7760 | 2: 28545094 | 7777 | 2: 28922483 |
| 7807 | 2: 29732106 | 7811 | 2: 29813146 | 7889 | 2: 31637856 | 7906 | 2: 32169593 | 8046 | 2: 36375875 |
| 8097 | 2: 38039323 | 8098 | 2: 38039898 | 8099 | 2: 38041017 | 8100 | 2: 38042315 | 8116 | 2: 38297286 |
| 8120 | 2: 38435225 | 8134 | 2: 38750534 | 8153 | 2: 39280801 | 8169 | 2: 39667440 | 8177 | 2: 39982069 |
| 8197 | 2: 40615263 | 8225 | 2: 41307524 | 8262 | 2: 42027595 | 8270 | 2: 42236387 | 8280 | 2: 42439089 |
| 8281 | 2: 42440990 | 8298 | 2: 42711951 | 8329 | 2: 43789161 | 8389 | 2: 45494226 | 8399 | 2: 45665663 |
| 8419 | 2: 46116917 | 8473 | 2: 47217427 | 8516 | 2: 48495171 | 8517 | 2: 48499243 | 8518 | 2: 48502118 |
| 8519 | 2: 48508248 | 8520 | 2: 48511801 | 8560 | 2: 49760042 | 8561 | 2: 49760735 | 8611 | 2: 51218510 |
| 8704 | 2: 53518329 | 8730 | 2: 54413562 | 8742 | 2: 54672099 | 8762 | 2: 55031934 | 8764 | 2: 55069900 |
| 8765 | 2: 55071242 | 8876 | 2: 58381419 | 8884 | 2: 58588299 | 8913 | 2: 59316972 | 8960 | 2: 60576956 |
| 8969 | 2: 60779135 | 9031 | 2: 62457322 | 9119 | 2: 64653571 | 9135 | 2: 64967881 | 9147 | 2: 65185518 |
| 9201 | 2: 66960608 | 9235 | 2: 68024571 | 9384 | 2: 72399848 | 9389 | 2: 72514078 | 9418 | 2: 73276155 |
| 9479 | 2: 74974797 | 9480 | 2: 74985065 | 9502 | 2: 75739779 | 9535 | 2: 76454912 | 9626 | 2: 78834490 |
| 9641 | 2: 79036468 | 9657 | 2: 79510060 | 9681 | 2: 80059990 | 9698 | 2: 80586285 | 9747 | 2: 83003800 |
| 9751 | 2: 83109314 | 9755 | 2: 83267596 | 9786 | 2: 83877880 | 9795 | 2: 84259043 | 9799 | 2: 84375486 |
| 9832 | 2: 85066770 | 9833 | 2: 85070174 | 9851 | 2: 85646050 | 9887 | 2: 86425660 | 9898 | 2: 86617347 |
| 9914 | 2: 87174608 | 9921 | 2: 87410708 | 9941 | 2: 88035286 | 9972 | 2: 88739478 | 10037 | 2: 89511031 |
| 10038 | 2: 89515068 | 10039 | 2: 89518986 | 10040 | 2: 89521529 | 10041 | 2: 89523615 | 10076 | 2: 90510385 |
| 10078 | 2: 90533958 | 10114 | 2: 91721645 | 10127 | 2: 92066999 | 10128 | 2: 92069312 | 10129 | 2: 92085813 |
| 10130 | 2: 92089545 | 10160 | 2: 92756276 | 10181 | 2: 93326093 | 10199 | 2: 93469951 | 10200 | 2: 93470548 |
| 10201 | 2: 93471304 | 10202 | 2: 93472560 | 10203 | 2: 93473816 | 10218 | 2: 93915068 | 10222 | 2: 94006514 |
| 10223 | 2: 94010942 | 10280 | 2: 95335798 | 10338 | 2: 96515244 | 10370 | 2: 97455980 | 10377 | 2: 97722574 |
| 10387 | 2: 97882323 | 10401 | 2: 98337087 | 10410 | 2: 98432307 | 10413 | 2: 98461459 | 10468 | 2: 99951594 |
| 10492 | 2: 100538824 | 10513 | 2: 101147607 | 10555 | 2: 102376439 | 10596 | 2: 103531207 | 10650 | 2: 104919346 |
| 10658 | 2: 105027822 | 10663 | 2: 105088054 | 10675 | 2: 105412051 | 10829 | 2: 108708703 | 10863 | 2: 109439523 |
| 11027 | 2: 113672892 | 11061 | 2: 114526247 | 11202 | 2: 118124674 | 11224 | 2: 118587782 | 11225 | 2: 118669657 |
| 11279 | 2: 119878226 | 11336 | 2: 121220519 | 11355 | 2: 121820038 | 11356 | 2: 121865885 | 11406 | 2: 123297173 |
| 11455 | 2: 124641406 | 11484 | 2: 125479428 | 11617 | 2: 127271059 | 11649 | 2: 127896093 | 11743 | 2: 130052119 |
| 11744 | 2: 130058714 | 11745 | 2: 130070890 | 11859 | 2: 132478188 | 11860 | 2: 132523448 | 11870 | 2: 132734429 |
| 11917 | 2: 133633752 | 11918 | 2: 133637575 | 11939 | 2: 133848234 | 11953 | 2: 134015104 | 12115 | 3: 525997 |
| 12118 | 3: 556754 | 12143 | 3: 950659 | 12145 | 3: 954886 | 12153 | 3: 1098608 | 12197 | 3: 1960686 |
| 12261 | 3: 3372085 | 12263 | 3: 3434999 | 12280 | 3: 3763587 | 12294 | 3: 4101542 | 12315 | 3: 4378889 |
| 12321 | 3: 4437644 | 12343 | 3: 4727841 | 12348 | 3: 4795643 | 12369 | 3: 5161089 | 12400 | 3: 5776030 |
| 12410 | 3: 6048242 | 12493 | 3: 7480890 | 12527 | 3: 7942262 | 12558 | 3: 8433782 | 12561 | 3: 8460244 |
| 12563 | 3: 8520080 | 12564 | 3: 8526638 | 12565 | 3: 8529009 | 12566 | 3: 8532372 | 12617 | 3: 10020876 |
| 12620 | 3: 10090524 | 12736 | 3: 11477492 | 12754 | 3: 11784186 | 12882 | 3: 14796654 | 12934 | 3: 15837312 |
| 13018 | 3: 17753548 | 13048 | 3: 18771436 | 13049 | 3: 18775018 | 13050 | 3: 18791656 | 13051 | 3: 18795346 |
| 13056 | 3: 18930543 | 13237 | 3: 23451211 | 13314 | 3: 25255575 | 13325 | 3: 25405368 | 13343 | 3: 25977712 |
| 13383 | 3: 26543166 | 13431 | 3: 27586116 | 13490 | 3: 28659529 | 13578 | 3: 30031426 | 13589 | 3: 30407397 |
| 13632 | 3: 31383515 | 13728 | 3: 32928510 | 13731 | 3: 32940407 | 13891 | 3: 35285151 | 13909 | 3: 35786483 |
| 13913 | 3: 35908304 | 13922 | 3: 36096832 | 13927 | 3: 36253878 | 13990 | 3: 37885414 | 14009 | 3: 38624906 |
| 14010 | 3: 38630748 | 14044 | 3: 39845695 | 14058 | 3: 40207827 | 14122 | 3: 42050876 | 14139 | 3: 42539907 |
| 14163 | 3: 42995977 | 14232 | 3: 44866434 | 14327 | 3: 47649002 | 14398 | 3: 49141481 | 14485 | 3: 50111050 |
| 14521 | 3: 51439192 | 14529 | 3: 51625928 | 14618 | 3: 53775526 | 14654 | 3: 55087957 | 14667 | 3: 55359809 |
| 14686 | 3: 56006266 | 14688 | 3: 56067950 | 14793 | 3: 59053277 | 14797 | 3: 59211837 | 14806 | 3: 59443494 |
| 14818 | 3: 59847747 | 14845 | 3: 60575455 | 14847 | 3: 60611151 | 14982 | 3: 64188398 | 15009 | 3: 65159985 |
| 15035 | 3: 65721928 | 15088 | 3: 66944634 | 15124 | 3: 67769302 | 15169 | 3: 68874408 | 15180 | 3: 69234087 |
| 15187 | 3: 69431230 | 15225 | 3: 70347328 | 15264 | 3: 72041156 | 15338 | 3: 74664014 | 15421 | 3: 77158063 |
| 15495 | 3: 79128870 | 15545 | 3: 80676731 | 15627 | 3: 82625118 | 15652 | 3: 83549724 | 15659 | 3: 83658003 |
| 15683 | 3: 84227201 | 15750 | 3: 85215781 | 15768 | 3: 85766170 | 15778 | 3: 85960283 | 15779 | 3: 85964163 |
| 15780 | 3: 85967104 | 15781 | 3: 85968892 | 15782 | 3: 85969726 | 15798 | 3: 86300913 | 15807 | 3: 86441083 |
| 5908 | 3: 88661891 | 15916 | 3: 88782949 | 15917 | 3: 88784034 | 15935 | 3: 89220299 | 15977 | 3: 89929116 |
| 16037 | 3: 91288695 | 16170 | 3: 94375264 | 16194 | 3: 95152254 | 16204 | 3: 95487322 | 16235 | 3: 96416999 |
| 16276 | 3: 97475924 | 16328 | 3: 98728026 | 16330 | 3: 98766843 | 16366 | 3: 99717720 | 16400 | 3: 100301019 |
| 16439 | 3: 100971847 | 16445 | 3: 101130049 | 16459 | 3: 101339407 | 16485 | 3: 101926220 | 16537 | 3: 102883747 |
| 16572 | 3: 103741473 | 16602 | 3: 104484207 | 16605 | 3: 104506752 | 16620 | 3: 104710861 | 16639 | 3: 105175424 |
| 16646 | 3: 105209686 | 16684 | 3: 105603312 | 16714 | 3: 105965567 | 16715 | 3: 105973059 | 16740 | 3: 106492826 |
| 16774 | 3: 107138615 | 16811 | 3: 107842370 | 16874 | 3: 109092493 | 16881 | 3: 109399680 | 16887 | 3: 109428951 |
| 16913 | 3: 109808282 | 16959 | 3: 110964139 | 17043 | 3: 112687905 | 17134 | 3: 114337662 | 17162 | 3: 114723123 |
| 17166 | 3: 114787475 | 17167 | 3: 114787630 | 17168 | 3: 114788049 | 17169 | 3: 114788632 | 17170 | 3: 114789131 |
| 17172 | 3: 114791529 | 17173 | 3: 114799492 | 17176 | 3: 114803442 | 17177 | 3: 114803451 | 17178 | 3: 114803465 |
| 17179 | 3: 114804161 | 17180 | 3: 114804367 | 17195 | 3: 115056511 | 17196 | 3: 115071383 | 17206 | 3: 115299586 |
| 17211 | 3: 115381373 | 17407 | 3: 118290616 | 17448 | 3: 118879237 | 17676 | 4: 4683962 | 17696 | 4: 5003827 |
| 17732 | 4: 5560351 | 17733 | 4: 5568155 | 17760 | 4: 6327312 | 17789 | 4: 7010182 | 17804 | 4: 7324916 |
| 17806 | 4: 7326126 | 17855 | 4: 8442378 | 17985 | 4: 10842025 | 18045 | 4: 12514592 | 18143 | 4: 15186933 |
| 18155 | 4: 15475039 | 18161 | 4: 15588873 | 18162 | 4: 15592728 | 18180 | 4: 16210509 | 18257 | 4: 18112247 |
| 18263 | 4: 18273472 | 18273 | 4: 18595000 | 18299 | 4: 18985118 | 18400 | 4: 20821405 | 18441 | 4: 21738699 |
| 18444 | 4: 21789346 | 18448 | 4: 21942101 | 18464 | 4: 22381610 | 18492 | 4: 22930826 | 18520 | 4: 23861032 |
| 18617 | 4: 26517471 | 18748 | 4: 30365149 | 18770 | 4: 30805170 | 18813 | 4: 31905587 | 18817 | 4: 31972387 |
| 18879 | 4: 33771936 | 18945 | 4: 35882843 | 19025 | 4: 38124757 | 19039 | 4: 38555013 | 19086 | 4: 40002914 |
| 19087 | 4: 40007566 | 19088 | 4: 40012097 | 19089 | 4: 40018785 | 19090 | 4: 40021130 | 19091 | 4: 40041810 |
| 19092 | 4: 40049842 | 19093 | 4: 40054177 | 19094 | 4: 40059057 | 19095 | 4: 40060306 | 19115 | 4: 40634211 |
| 19116 | 4: 40640519 | 19117 | 4: 40645360 | 19118 | 4: 40649968 | 19119 | 4: 40654713 | 19167 | 4: 41814723 |
| 19172 | 4: 42014141 | 19183 | 4: 42356867 | 19190 | 4: 42554917 | 19255 | 4: 44244202 | 19291 | 4: 45415922 |
| 19296 | 4: 45536174 | 19308 | 4: 45825175 | 19335 | 4: 46718903 | 19342 | 4: 46848225 | 19357 | 4: 47216690 |
| 19358 | 4: 47217662 | 19359 | 4: 47221067 | 19360 | 4: 47224473 | 19361 | 4: 47228083 | 19400 | 4: 48100380 |
| 19463 | 4: 49408307 | 19575 | 4: 53093575 | 19600 | 4: 53722558 | 19607 | 4: 53807295 | 19720 | 4: 57434269 |
| 19763 | 4: 58913134 | 19783 | 4: 59391253 | 19787 | 4: 59489126 | 19813 | 4: 60378132 | 19818 | 4: 60482892 |
| 19853 | 4: 61414628 | 19854 | 4: 61423853 | 19855 | 4: 61432230 | 19856 | 4: 61434936 | 19857 | 4: 61439997 |
| 19881 | 4: 62128232 | 19885 | 4: 62171821 | 19916 | 4: 63134475 | 19951 | 4: 63940606 | 19960 | 4: 64270176 |
| 19961 | 4: 64271209 | 19964 | 4: 64495002 | 19995 | 4: 65572773 | 20043 | 4: 66416290 | 20063 | 4: 67092276 |
| 20082 | 4: 67523624 | 20114 | 4: 68300881 | 20190 | 4: 70507936 | 20193 | 4: 70569760 | 20246 | 4: 72066682 |
| 20297 | 4: 73725730 | 20302 | 4: 73904776 | 20343 | 4: 74843205 | 20361 | 4: 75190744 | 20382 | 4: 75792193 |
| 20390 | 4: 75908626 | 20442 | 4: 77149758 | 20462 | 4: 77552330 | 20482 | 4: 77954957 | 20489 | 4: 78153455 |
| 20519 | 4: 79093225 | 20523 | 4: 79236024 | 20527 | 4: 79350296 | 20537 | 4: 79637506 | 20607 | 4: 81119261 |
| 20687 | 4: 83090213 | 20854 | 4: 87615934 | 20860 | 4: 87832774 | 20862 | 4: 87887042 | 20866 | 4: 87931932 |
| 20867 | 4: 87937433 | 20918 | 4: 89596848 | 20927 | 4: 89756186 | 20933 | 4: 89854090 | 20952 | 4: 90405270 |
| 20965 | 4: 90752463 | 20983 | 4: 91186390 | 21013 | 4: 91662043 | 21020 | 4: 91835951 | 21026 | 4: 91985689 |
| 21064 | 4: 92683225 | 21143 | 4: 94586866 | 21162 | 4: 95121580 | 21208 | 4: 95976133 | 21209 | 4: 95978012 |
| 21210 | 4: 95984102 | 21211 | 4: 95986472 | 21212 | 4: 95988223 | 21213 | 4: 95991905 | 21214 | 4: 95994577 |
| 21215 | 4: 95996626 | 21216 | 4: 95999064 | 21217 | 4: 95999599 | 21218 | 4: 95999657 | 21220 | 4: 96006244 |
| 21223 | 4: 96012342 | 21224 | 4: 96014135 | 21225 | 4: 96014452 | 21226 | 4: 96014495 | 21229 | 4: 96043384 |
| 21230 | 4: 96043881 | 21258 | 4: 96806394 | 21259 | 4: 96808296 | 21260 | 4: 96808305 | 21261 | 4: 96808793 |
| 21263 | 4: 96810164 | 21264 | 4: 96810688 | 21265 | 4: 96810739 | 21266 | 4: 96811491 | 21267 | 4: 96811492 |
| 21268 | 4: 96811663 | 21269 | 4: 96819713 | 21270 | 4: 96819807 | 21271 | 4: 96819821 | 21272 | 4: 96820983 |
| 21273 | 4: 96821494 | 21274 | 4: 96822544 | 21275 | 4: 96822709 | 21276 | 4: 96822722 | 21277 | 4: 96823395 |
| 21278 | 4: 96823570 | 21280 | 4: 96826910 | 21281 | 4: 96827344 | 21282 | 4: 96828046 | 21283 | 4: 96828753 |
| 21284 | 4: 96829524 | 21286 | 4: 96842438 | 21287 | 4: 96842474 | 21288 | 4: 96846828 | 21289 | 4: 96847175 |
| 21290 | 4: 96847270 | 21291 | 4: 96847298 | 21292 | 4: 96847522 | 21293 | 4: 96847685 | 21294 | 4: 96848163 |
| 21295 | 4: 96852063 | 21296 | 4: 96852112 | 21297 | 4: 96852128 | 21299 | 4: 96853114 | 21300 | 4: 96853149 |
| 21301 | 4: 96854041 | 21302 | 4: 96854052 | 21303 | 4: 96854217 | 21304 | 4: 96854309 | 21305 | 4: 96854674 |
| 21306 | 4: 96854753 | 21307 | 4: 96854840 | 21308 | 4: 96854846 | 21309 | 4: 96855073 | 21310 | 4: 96855142 |
| 21311 | 4: 96855211 | 21312 | 4: 96855234 | 21313 | 4: 96855382 | 21314 | 4: 96858356 | 21315 | 4: 96858411 |
| 21316 | 4: 96858620 | 21317 | 4: 96859305 | 21318 | 4: 96859677 | 21319 | 4: 96859857 | 21320 | 4: 96860289 |
| 21321 | 4: 96860821 | 21322 | 4: 96861792 | 21323 | 4: 96862653 | 21324 | 4: 96868903 | 21325 | 4: 96870132 |
| 21326 | 4: 96870621 | 21327 | 4: 96871036 | 21328 | 4: 96871329 | 21329 | 4: 96872714 | 21330 | 4: 96872734 |
| 21331 | 4: 96878187 | 21332 | 4: 96878242 | 21333 | 4: 96878278 | 21334 | 4: 96880890 | 21335 | 4: 96880962 |
| 21336 | 4: 96881026 | 21337 | 4: 96881161 | 21338 | 4: 96881966 | 21339 | 4: 96882244 | 21340 | 4: 96885170 |
| 21341 | 4: 96886853 | 21342 | 4: 96887160 | 21343 | 4: 96887442 | 21344 | 4: 96887486 | 21345 | 4: 96887810 |
| 21346 | 4: 96888595 | 21347 | 4: 96889148 | 21348 | 4: 96890359 | 21349 | 4: 96890450 | 21350 | 4: 96890701 |
| 21351 | 4: 96891069 | 21352 | 4: 96891335 | 21353 | 4: 96891542 | 21354 | 4: 96891552 | 21355 | 4: 96891608 |
| 21356 | 4: 96895620 | 21357 | 4: 96897050 | 21358 | 4: 96897145 | 21367 | 4: 97039076 | 21370 | 4: 97088268 |
| 21372 | 4: 97137855 | 21373 | 4: 97147752 | 21374 | 4: 97148005 | 21375 | 4: 97148878 | 21376 | 4: 97150124 |
| 21377 | 4: 97150300 | 21378 | 4: 97151421 | 21379 | 4: 97151944 | 21380 | 4: 97152001 | 21381 | 4: 97152026 |
| 21382 | 4: 97152080 | 21383 | 4: 97152151 | 21384 | 4: 97152177 | 21385 | 4: 97152300 | 21386 | 4: 97152327 |
| 21387 | 4: 97152343 | 21388 | 4: 97152464 | 21389 | 4: 97152562 | 21390 | 4: 97152880 | 21391 | 4: 97153058 |
| 21392 | 4: 97160379 | 21393 | 4: 97160479 | 21394 | 4: 97160507 | 21395 | 4: 97160725 | 21396 | 4: 97160764 |
| 21397 | 4: 97160849 | 21398 | 4: 97160967 | 21399 | 4: 97161387 | 21400 | 4: 97161554 | 21401 | 4: 97161881 |
| 21402 | 4: 97161958 | 21404 | 4: 97164436 | 21405 | 4: 97165222 | 21406 | 4: 97165446 | 21407 | 4: 97165671 |
| 21408 | 4: 97165867 | 21409 | 4: 97167273 | 21410 | 4: 97170350 | 21411 | 4: 97170722 | 21412 | 4: 97170758 |
| 21413 | 4: 97170786 | 21415 | 4: 97171793 | 21417 | 4: 97173637 | 21418 | 4: 97173882 | 21420 | 4: 97243175 |
| 21429 | 4: 97478233 | 21449 | 4: 97997496 | 21460 | 4: 98301073 | 21488 | 4: 99227319 | 21496 | 4: 99551602 |
| 21525 | 4: 100096858 | 21571 | 4: 101147322 | 21621 | 4: 102291227 | 21665 | 4: 103373158 | 21709 | 4: 104494825 |
| 21712 | 4: 104541226 | 21740 | 4: 105243840 | 21826 | 4: 106811124 | 21828 | 4: 106840758 | 21903 | 4: 108136633 |
| 21927 | 4: 108959151 | 21976 | 4: 110025220 | 22029 | 4: 111137956 | 22031 | 4: 111174901 | 22032 | 4: 111185441 |
| 22052 | 4: 111594642 | 22057 | 4: 111656507 | 22081 | 4: 112008507 | 22182 | 4: 113533731 | 22254 | 4: 114468144 |
| 22334 | 4: 115504063 | 22339 | 4: 115674188 | 22348 | 4: 115903138 | 22484 | 4: 118639815 | 22639 | 5: 1916328 |
| 22664 | 5: 2361885 | 22679 | 5: 2678940 | 22680 | 5: 2710438 | 22682 | 5: 2775410 | 22683 | 5: 2789169 |
| 22692 | 5: 3084344 | 22721 | 5: 4106293 | 22747 | 5: 4642231 | 22801 | 5: 5757045 | 22812 | 5: 5962601 |
| 22864 | 5: 7150201 | 22875 | 5: 7320087 | 22901 | 5: 8063807 | 22907 | 5: 8295061 | 22914 | 5: 8547280 |
| 23001 | 5: 10417113 | 23005 | 5: 10483003 | 23116 | 5: 12929697 | 23147 | 5: 13814938 | 23154 | 5: 14118702 |
| 23200 | 5: 15181434 | 23209 | 5: 15382347 | 23335 | 5: 18879061 | 23345 | 5: 19180052 | 23365 | 5: 19703644 |
| 23420 | 5: 21141526 | 23424 | 5: 21247771 | 23441 | 5: 21652414 | 23488 | 5: 22524662 | 23553 | 5: 23856635 |
| 23577 | 5: 24327813 | 23580 | 5: 24363604 | 23609 | 5: 25025028 | 23638 | 5: 25910383 | 23667 | 5: 26553269 |
| 23765 | 5: 28658495 | 23768 | 5: 28689345 | 23795 | 5: 29481439 | 23827 | 5: 30068971 | 23860 | 5: 30880544 |
| 23885 | 5: 31649328 | 23903 | 5: 32253508 | 23967 | 5: 33731249 | 23969 | 5: 33743644 | 23990 | 5: 34527357 |
| 24023 | 5: 35215343 | 24041 | 5: 35917350 | 24064 | 5: 36532219 | 24164 | 5: 39219011 | 24213 | 5: 40323694 |
| 24252 | 5: 41581614 | 24255 | 5: 41737754 | 24277 | 5: 42488185 | 24295 | 5: 42883310 | 24309 | 5: 43239107 |
| 24321 | 5: 43556568 | 24332 | 5: 43826757 | 24373 | 5: 44749275 | 24394 | 5: 45422250 | 24412 | 5: 45892148 |
| 24469 | 5: 47071100 | 24474 | 5: 47204283 | 24476 | 5: 47210190 | 24491 | 5: 47544371 | 24515 | 5: 48255910 |
| 24533 | 5: 48697507 | 24700 | 5: 53392371 | 24725 | 5: 54050541 | 24767 | 5: 54925075 | 24868 | 5: 57315947 |
| 24869 | 5: 57316513 | 24870 | 5: 57318993 | 24931 | 5: 59393649 | 25012 | 5: 61592328 | 25049 | 5: 62915639 |
| 25065 | 5: 63217741 | 25070 | 5: 63272705 | 25094 | 5: 63826831 | 25189 | 5: 66342506 | 25206 | 5: 66729461 |
| 25248 | 5: 67796979 | 25274 | 5: 68252711 | 25308 | 5: 69044504 | 25336 | 5: 69658212 | 25386 | 5: 70977990 |
| 25396 | 5: 71157250 | 25405 | 5: 71284786 | 25414 | 5: 71441252 | 25415 | 5: 71451984 | 25452 | 5: 72240212 |
| 25455 | 5: 72267018 | 25501 | 5: 73577352 | 25505 | 5: 73676579 | 25512 | 5: 73764801 | 25516 | 5: 73813825 |
| 25528 | 5: 74101395 | 25540 | 5: 74482047 | 25592 | 5: 75448318 | 25633 | 5: 76668839 | 25639 | 5: 76766433 |
| 25644 | 5: 76890153 | 25683 | 5: 77996585 | 25684 | 5: 78020028 | 25716 | 5: 78821705 | 25736 | 5: 79345129 |
| 25744 | 5: 79644695 | 25768 | 5: 80587434 | 25796 | 5: 81407773 | 25800 | 5: 81437991 | 25801 | 5: 81441963 |
| 25851 | 5: 82570119 | 25855 | 5: 82593018 | 25856 | 5: 82593228 | 25857 | 5: 82593982 | 25858 | 5: 82594019 |
| 25859 | 5: 82594028 | 25860 | 5: 82594375 | 25861 | 5: 82594380 | 25862 | 5: 82595065 | 25863 | 5: 82595148 |
| 25864 | 5: 82595334 | 25865 | 5: 82627761 | 25905 | 5: 83646235 | 26064 | 5: 84298547 | 26072 | 5: 84433271 |
| 26074 | 5: 84527341 | 26089 | 5: 84948836 | 26145 | 5: 86376332 | 26148 | 5: 86455776 | 26196 | 5: 87435829 |
| 26293 | 5: 88856288 | 26361 | 5: 90566853 | 26403 | 5: 91501158 | 26409 | 5: 91665913 | 26415 | 5: 91761326 |
| 26432 | 5: 92332808 | 26496 | 5: 93178439 | 26526 | 5: 93790346 | 26533 | 5: 93961735 | 26537 | 5: 94088171 |
| 26579 | 5: 95091626 | 26625 | 5: 96054240 | 26685 | 5: 97071869 | 26781 | 5: 98982241 | 26878 | 5: 100843102 |
| 26891 | 5: 101049907 | 26948 | 5: 102626302 | 27117 | 5: 105882827 | 27183 | 5: 106801714 | 27187 | 5: 106877986 |
| 27206 | 5: 107060379 | 27244 | 5: 107745197 | 27305 | 5: 108794780 | 27306 | 5: 108797633 | 27341 | 5: 109446738 |
| 27366 | 5: 109926959 | 27428 | 5: 111036587 | 27579 | 5: 114683035 | 27584 | 5: 114814526 | 27611 | 5: 115497329 |
| 27613 | 5: 115504227 | 27623 | 5: 115614570 | 27664 | 5: 116449909 | 27776 | 5: 117284799 | 27782 | 5: 117411822 |
| 27811 | 5: 117753977 | 27824 | 5: 117977735 | 27839 | 5: 118288125 | 27856 | 5: 118666844 | 27917 | 5: 119642118 |
| 27951 | 6: 579203 | 28082 | 6: 3248784 | 28098 | 6: 3406400 | 28122 | 6: 3655995 | 28164 | 6: 4606383 |
| 28201 | 6: 5673122 | 28210 | 6: 5813338 | 28269 | 6: 6797112 | 28271 | 6: 6808370 | 28384 | 6: 9740976 |
| 28388 | 6: 9896356 | 28418 | 6: 10695206 | 28487 | 6: 12244147 | 28494 | 6: 12408823 | 28501 | 6: 12515175 |
| 28516 | 6: 12899581 | 28534 | 6: 13157939 | 28535 | 6: 13158398 | 28536 | 6: 13158702 | 28570 | 6: 14149976 |
| 28645 | 6: 15766628 | 28649 | 6: 15875705 | 28664 | 6: 16165237 | 28720 | 6: 17350197 | 28729 | 6: 17546682 |
| 28775 | 6: 18247972 | 28798 | 6: 18858161 | 28832 | 6: 19517669 | 28855 | 6: 20067345 | 28903 | 6: 21187469 |
| 28951 | 6: 22198236 | 28974 | 6: 22627641 | 28985 | 6: 22838005 | 28994 | 6: 23185445 | 29012 | 6: 23551846 |
| 29033 | 6: 24079559 | 29091 | 6: 25420968 | 29092 | 6: 25430886 | 29113 | 6: 25987389 | 29124 | 6: 26122133 |
| 29169 | 6: 27357262 | 29187 | 6: 27922767 | 29228 | 6: 29044156 | 29256 | 6: 29526067 | 29268 | 6: 29755371 |
| 29360 | 6: 30504972 | 29389 | 6: 31372994 | 29391 | 6: 31402252 | 29427 | 6: 32495711 | 29446 | 6: 33106068 |
| 29505 | 6: 34316541 | 29908 | 6: 34880169 | 29988 | 6: 35895370 | 29990 | 6: 35897546 | 29991 | 6: 35897939 |
| 29992 | 6: 35906185 | 29993 | 6: 35907046 | 29995 | 6: 35909397 | 29998 | 6: 35925489 | 30025 | 6: 36097722 |
| 30135 | 6: 36427342 | 30137 | 6: 36430013 | 30140 | 6: 36432356 | 30143 | 6: 36441375 | 30257 | 6: 36563183 |
| 30260 | 6: 36568261 | 30264 | 6: 36572899 | 30333 | 6: 36813124 | 30455 | 6: 37026863 | 30456 | 6: 37027429 |
| 30457 | 6: 37027539 | 30458 | 6: 37028623 | 30460 | 6: 37030480 | 30461 | 6: 37032906 | 30462 | 6: 37032995 |
| 30467 | 6: 37035186 | 30469 | 6: 37038017 | 30471 | 6: 37056413 | 30475 | 6: 37075910 | 30566 | 6: 37095299 |
| 30616 | 6: 37107589 | 30626 | 6: 37117771 | 30629 | 6: 37121529 | 30630 | 6: 37123023 | 30632 | 6: 37130785 |
| 30633 | 6: 37134418 | 30636 | 6: 37136794 | 30643 | 6: 37142174 | 30649 | 6: 37149945 | 30685 | 6: 37161408 |
| 30736 | 6: 37173048 | 30788 | 6: 37184577 | 30791 | 6: 37188623 | 30792 | 6: 37189736 | 30798 | 6: 37206928 |
| 30799 | 6: 37210690 | 30802 | 6: 37214389 | 30803 | 6: 37214736 | 30810 | 6: 37219609 | 30813 | 6: 37221776 |
| 30818 | 6: 37227250 | 30820 | 6: 37231623 | 30829 | 6: 37235373 | 30830 | 6: 37235374 | 30831 | 6: 37236226 |
| 30844 | 6: 37244478 | 30849 | 6: 37247874 | 30851 | 6: 37250850 | 30852 | 6: 37251132 | 30854 | 6: 37252978 |
| 30856 | 6: 37256488 | 30857 | 6: 37260221 | 30858 | 6: 37262825 | 30859 | 6: 37262995 | 30864 | 6: 37269061 |
| 30865 | 6: 37270215 | 30866 | 6: 37270930 | 30867 | 6: 37271635 | 30870 | 6: 37281308 | 30871 | 6: 37281335 |
| 30872 | 6: 37282356 | 30873 | 6: 37284874 | 30879 | 6: 37286627 | 30880 | 6: 37286722 | 30881 | 6: 37287306 |
| 30882 | 6: 37287423 | 30883 | 6: 37287873 | 30884 | 6: 37288219 | 30885 | 6: 37288483 | 30886 | 6: 37288600 |
| 30887 | 6: 37288948 | 30888 | 6: 37289071 | 30889 | 6: 37289572 | 30890 | 6: 37289742 | 30891 | 6: 37289905 |
| 30892 | 6: 37289990 | 30893 | 6: 37290180 | 30894 | 6: 37290241 | 30895 | 6: 37290415 | 30896 | 6: 37291360 |
| 30897 | 6: 37291500 | 30898 | 6: 37292117 | 30899 | 6: 37292269 | 30900 | 6: 37292270 | 30901 | 6: 37292298 |
| 30902 | 6: 37292752 | 30903 | 6: 37293476 | 30905 | 6: 37294505 | 30913 | 6: 37297426 | 30917 | 6: 37298725 |
| 30927 | 6: 37300810 | 30932 | 6: 37301959 | 30936 | 6: 37302499 | 30938 | 6: 37303255 | 30946 | 6: 37304513 |
| 30952 | 6: 37305466 | 30956 | 6: 37306944 | 30959 | 6: 37309266 | 30960 | 6: 37310413 | 30972 | 6: 37313503 |
| 30977 | 6: 37314183 | 30993 | 6: 37317695 | 31003 | 6: 37321049 | 31004 | 6: 37321441 | 31010 | 6: 37324718 |
| 31018 | 6: 37328528 | 31026 | 6: 37329878 | 31030 | 6: 37330306 | 31032 | 6: 37330477 | 31033 | 6: 37330577 |
| 31036 | 6: 37331712 | 31046 | 6: 37335926 | 31052 | 6: 37337744 | 31056 | 6: 37340937 | 31058 | 6: 37342568 |
| 31066 | 6: 37345508 | 31070 | 6: 37347130 | 31071 | 6: 37349144 | 31075 | 6: 37351767 | 31076 | 6: 37352145 |
| 31086 | 6: 37359765 | 31088 | 6: 37360685 | 31089 | 6: 37361449 | 31090 | 6: 37361578 | 31091 | 6: 37362262 |
| 31099 | 6: 37369338 | 31100 | 6: 37369380 | 31102 | 6: 37372334 | 31108 | 6: 37376289 | 31111 | 6: 37378475 |
| 31113 | 6: 37379506 | 31114 | 6: 37379507 | 31118 | 6: 37380161 | 31120 | 6: 37381106 | 31122 | 6: 37381782 |
| 31123 | 6: 37382003 | 31124 | 6: 37382062 | 31127 | 6: 37383526 | 31129 | 6: 37385973 | 31133 | 6: 37391915 |
| 31138 | 6: 37393949 | 31139 | 6: 37394877 | 31140 | 6: 37395313 | 31143 | 6: 37396789 | 31144 | 6: 37398199 |
| 31145 | 6: 37399560 | 31147 | 6: 37400741 | 31151 | 6: 37403131 | 31152 | 6: 37403224 | 31153 | 6: 37406239 |
| 31154 | 6: 37406485 | 31156 | 6: 37407653 | 31157 | 6: 37412062 | 31162 | 6: 37415367 | 31163 | 6: 37416259 |
| 31166 | 6: 37417501 | 31167 | 6: 37418164 | 31169 | 6: 37418447 | 31170 | 6: 37418449 | 31172 | 6: 37420957 |
| 31174 | 6: 37424248 | 31176 | 6: 37429071 | 31178 | 6: 37429776 | 31179 | 6: 37430447 | 31180 | 6: 37431101 |
| 31181 | 6: 37432458 | 31186 | 6: 37435861 | 31188 | 6: 37436347 | 31191 | 6: 37437538 | 31192 | 6: 37438222 |
| 31197 | 6: 37438993 | 31199 | 6: 37440571 | 31204 | 6: 37444900 | 31208 | 6: 37449890 | 31212 | 6: 37457249 |
| 31216 | 6: 37465669 | 31217 | 6: 37466194 | 31218 | 6: 37473453 | 31219 | 6: 37477629 | 31221 | 6: 37480277 |
| 31222 | 6: 37482054 | 31225 | 6: 37491305 | 31229 | 6: 37503406 | 31230 | 6: 37505093 | 31231 | 6: 37508184 |
| 31235 | 6: 37513645 | 31239 | 6: 37522580 | 31240 | 6: 37526812 | 31243 | 6: 37534297 | 31246 | 6: 37553481 |
| 31248 | 6: 37560625 | 31249 | 6: 37562383 | 31251 | 6: 37566268 | 31252 | 6: 37566317 | 31254 | 6: 37573307 |
| 31255 | 6: 37573615 | 31256 | 6: 37578832 | 31262 | 6: 37587980 | 31263 | 6: 37588492 | 31264 | 6: 37591326 |
| 31265 | 6: 37591664 | 31268 | 6: 37598284 | 31276 | 6: 37608504 | 31278 | 6: 37614179 | 31280 | 6: 37624003 |
| 31281 | 6: 37625923 | 31282 | 6: 37627776 | 31284 | 6: 37636883 | 31285 | 6: 37636900 | 31286 | 6: 37637484 |
| 31287 | 6: 37639651 | 31288 | 6: 37639763 | 31289 | 6: 37640954 | 31290 | 6: 37641448 | 31291 | 6: 37641798 |
| 31292 | 6: 37642775 | 31293 | 6: 37644691 | 31294 | 6: 37644915 | 31295 | 6: 37645671 | 31296 | 6: 37646000 |
| 31297 | 6: 37646499 | 31301 | 6: 37658848 | 31349 | 6: 37772675 | 31353 | 6: 37774778 | 31355 | 6: 37775624 |
| 31357 | 6: 37776401 | 31359 | 6: 37776454 | 31360 | 6: 37776464 | 31361 | 6: 37777029 | 31363 | 6: 37777185 |
| 31404 | 6: 37817575 | 31438 | 6: 37912116 | 31445 | 6: 37914472 | 31454 | 6: 37917366 | 31455 | 6: 37917421 |
| 31465 | 6: 37919853 | 31475 | 6: 37926073 | 31476 | 6: 37926600 | 31477 | 6: 37926960 | 31478 | 6: 37928156 |
| 31484 | 6: 37929786 | 31486 | 6: 37930252 | 31487 | 6: 37930293 | 31488 | 6: 37930672 | 31490 | 6: 37930868 |
| 31491 | 6: 37930889 | 31492 | 6: 37931089 | 31493 | 6: 37931216 | 31494 | 6: 37931298 | 31495 | 6: 37931621 |
| 31496 | 6: 37931688 | 31499 | 6: 37932170 | 31501 | 6: 37932241 | 31503 | 6: 37932377 | 31504 | 6: 37932412 |
| 31506 | 6: 37932563 | 31507 | 6: 37932682 | 31508 | 6: 37932772 | 31509 | 6: 37932778 | 31510 | 6: 37932960 |
| 31512 | 6: 37933352 | 31514 | 6: 37934068 | 31518 | 6: 37935108 | 31519 | 6: 37935168 | 31520 | 6: 37935527 |
| 31521 | 6: 37935577 | 31522 | 6: 37935748 | 31524 | 6: 37936087 | 31525 | 6: 37936692 | 31526 | 6: 37938294 |
| 31527 | 6: 37938374 | 31528 | 6: 37938826 | 31531 | 6: 37942290 | 31532 | 6: 37942944 | 31533 | 6: 37943635 |
| 31534 | 6: 37943860 | 31535 | 6: 37943970 | 31536 | 6: 37944478 | 31537 | 6: 37944482 | 31538 | 6: 37944496 |
| 31539 | 6: 37944573 | 31541 | 6: 37944681 | 31542 | 6: 37944704 | 31544 | 6: 37945482 | 31545 | 6: 37945820 |
| 31546 | 6: 37945849 | 31558 | 6: 37959360 | 31561 | 6: 37961553 | 31563 | 6: 37962887 | 31565 | 6: 37963525 |
| 31568 | 6: 37966382 | 31585 | 6: 37974401 | 31586 | 6: 37974407 | 31819 | 6: 38395086 | 31822 | 6: 38409517 |
| 31826 | 6: 38491475 | 31831 | 6: 38518347 | 31836 | 6: 38540452 | 31837 | 6: 38541525 | 31839 | 6: 38542048 |
| 31842 | 6: 38543400 | 31843 | 6: 38543545 | 31844 | 6: 38543990 | 31845 | 6: 38545112 | 31846 | 6: 38545113 |
| 31847 | 6: 38546290 | 31848 | 6: 38546646 | 31849 | 6: 38548060 | 31850 | 6: 38548198 | 31851 | 6: 38548496 |
| 31852 | 6: 38548525 | 31853 | 6: 38548659 | 31854 | 6: 38549044 | 31855 | 6: 38549099 | 31856 | 6: 38549206 |
| 31857 | 6: 38549477 | 31859 | 6: 38550162 | 31860 | 6: 38550667 | 31861 | 6: 38552085 | 31862 | 6: 38552158 |
| 31863 | 6: 38552167 | 31864 | 6: 38552459 | 31865 | 6: 38552550 | 31866 | 6: 38552644 | 31867 | 6: 38552821 |
| 31868 | 6: 38553028 | 31869 | 6: 38553181 | 31870 | 6: 38553447 | 31871 | 6: 38553456 | 31872 | 6: 38553565 |
| 31873 | 6: 38553842 | 31874 | 6: 38553946 | 31875 | 6: 38554100 | 31876 | 6: 38554163 | 31877 | 6: 38554439 |
| 31878 | 6: 38554455 | 31879 | 6: 38554714 | 31880 | 6: 38555555 | 31881 | 6: 38556220 | 31882 | 6: 38556748 |
| 31883 | 6: 38556956 | 31884 | 6: 38556978 | 31885 | 6: 38557241 | 31886 | 6: 38557498 | 31887 | 6: 38557633 |
| 31888 | 6: 38557694 | 31889 | 6: 38557830 | 31890 | 6: 38557833 | 31891 | 6: 38557888 | 31892 | 6: 38557940 |
| 31894 | 6: 38558946 | 31895 | 6: 38562229 | 31897 | 6: 38564166 | 31898 | 6: 38565084 | 31902 | 6: 38583433 |
| 31904 | 6: 38584686 | 31908 | 6: 38600235 | 31965 | 6: 39250952 | 31969 | 6: 39301737 | 31989 | 6: 39663383 |
| 31992 | 6: 39721697 | 31993 | 6: 39727899 | 31994 | 6: 39730068 | 31995 | 6: 39733610 | 31996 | 6: 39737879 |
| 32029 | 6: 40129309 | 32034 | 6: 40237863 | 32062 | 6: 40696320 | 32086 | 6: 40735514 | 32119 | 6: 40952449 |
| 32207 | 6: 42123221 | 32266 | 6: 43009159 | 32267 | 6: 43017852 | 32294 | 6: 43462079 | 32329 | 6: 43973189 |
| 32339 | 6: 44149286 | 32547 | 6: 46220665 | 32588 | 6: 47190072 | 32619 | 6: 47907662 | 32631 | 6: 48281817 |
| 32657 | 6: 49188655 | 32670 | 6: 49492652 | 32675 | 6: 49628351 | 32678 | 6: 49692019 | 32757 | 6: 51655726 |
| 32824 | 6: 52798083 | 32827 | 6: 52912323 | 32854 | 6: 53474037 | 32911 | 6: 55287740 | 32947 | 6: 55553127 |
| 32949 | 6: 55563964 | 32951 | 6: 55573386 | 32968 | 6: 56049754 | 32972 | 6: 56230949 | 33026 | 6: 57612544 |
| 33034 | 6: 57940998 | 33036 | 6: 57996603 | 33038 | 6: 58046666 | 33086 | 6: 59279953 | 33101 | 6: 59756158 |
| 33144 | 6: 60551717 | 33151 | 6: 60729399 | 33174 | 6: 61339528 | 33199 | 6: 61996360 | 33215 | 6: 62563366 |
| 33218 | 6: 62627183 | 33317 | 6: 65798913 | 33363 | 6: 67312644 | 33364 | 6: 67348632 | 33404 | 6: 68600503 |
| 33421 | 6: 69030371 | 33437 | 6: 69395469 | 33438 | 6: 69429025 | 33455 | 6: 70069795 | 33480 | 6: 70868126 |
| 33492 | 6: 71178118 | 33506 | 6: 71614584 | 33509 | 6: 71658863 | 33575 | 6: 73737226 | 33611 | 6: 75157043 |
| 33635 | 6: 75976163 | 33715 | 6: 78625022 | 33873 | 6: 83484610 | 33909 | 6: 84465355 | 33927 | 6: 84898498 |
| 34137 | 6: 89248038 | 34177 | 6: 90260850 | 34180 | 6: 90360832 | 34292 | 6: 92070503 | 34356 | 6: 93561855 |
| 34416 | 6: 94977849 | 34431 | 6: 95495492 | 34452 | 6: 96090013 | 34453 | 6: 96097237 | 34476 | 6: 96479912 |
| 34478 | 6: 96544331 | 34486 | 6: 96686083 | 34491 | 6: 96916348 | 34531 | 6: 97462721 | 34545 | 6: 97701064 |
| 34600 | 6: 98712630 | 34635 | 6: 99753620 | 34663 | 6: 100291469 | 34716 | 6: 101484421 | 34717 | 6: 101486616 |
| 34720 | 6: 101524578 | 34735 | 6: 101873336 | 34761 | 6: 102268756 | 34817 | 6: 103176226 | 34828 | 6: 103236460 |
| 34912 | 6: 105003956 | 35080 | 6: 109427077 | 35101 | 6: 109822389 | 35163 | 6: 111118988 | 35165 | 6: 111196630 |
| 35175 | 6: 111382228 | 35229 | 6: 112604266 | 35271 | 6: 113397576 | 35275 | 6: 113470683 | 35406 | 6: 115996386 |
| 35448 | 6: 116540874 | 35480 | 7: 310937 | 35482 | 7: 325799 | 35558 | 7: 1927986 | 35561 | 7: 1995591 |
| 35582 | 7: 2406915 | 35845 | 7: 6222553 | 35873 | 7: 6554748 | 35887 | 7: 6745523 | 35900 | 7: 6956627 |
| 35928 | 7: 7534130 | 35936 | 7: 7674523 | 35947 | 7: 8035405 | 35989 | 7: 10310655 | 35990 | 7: 10395846 |
| 36049 | 7: 11976493 | 36068 | 7: 12333943 | 36069 | 7: 12335290 | 36140 | 7: 13300617 | 36153 | 7: 13562003 |
| 36173 | 7: 13942658 | 36277 | 7: 15601726 | 36310 | 7: 16447387 | 36311 | 7: 16487854 | 36362 | 7: 17629628 |
| 36364 | 7: 17639856 | 36392 | 7: 18356451 | 36423 | 7: 19107522 | 36440 | 7: 19307460 | 36503 | 7: 20153088 |
| 36525 | 7: 20401996 | 36546 | 7: 20912796 | 36597 | 7: 22016441 | 36603 | 7: 22057135 | 36620 | 7: 22504243 |
| 36646 | 7: 23221425 | 36663 | 7: 23603215 | 36683 | 7: 24060066 | 36685 | 7: 24072015 | 36705 | 7: 24634875 |
| 36768 | 7: 26043336 | 36828 | 7: 27103526 | 36858 | 7: 27828159 | 36879 | 7: 28407768 | 36890 | 7: 28661620 |
| 36941 | 7: 29715806 | 37014 | 7: 31333882 | 37042 | 7: 31763022 | 37047 | 7: 31806849 | 37062 | 7: 32255462 |
| 37091 | 7: 33083761 | 37108 | 7: 33452281 | 37175 | 7: 35015402 | 37188 | 7: 35370637 | 37211 | 7: 35997017 |
| 37230 | 7: 36507816 | 37294 | 7: 37977197 | 37369 | 7: 39696107 | 37371 | 7: 39720514 | 37528 | 7: 43355224 |
| 37544 | 7: 43621545 | 37609 | 7: 45530009 | 37635 | 7: 46401887 | 37725 | 7: 48628543 | 37738 | 7: 48808046 |
| 37745 | 7: 48945061 | 37752 | 7: 49169638 | 37886 | 7: 52686851 | 37912 | 7: 53315105 | 37957 | 7: 54541065 |
| 37960 | 7: 54622430 | 37975 | 7: 55088638 | 38007 | 7: 55900961 | 38015 | 7: 56078682 | 38033 | 7: 56535413 |
| 38035 | 7: 56546344 | 38057 | 7: 57273769 | 38076 | 7: 57910079 | 38077 | 7: 57916334 | 38081 | 7: 57993375 |
| 38083 | 7: 58043785 | 38153 | 7: 59374018 | 38159 | 7: 59461244 | 38188 | 7: 60116908 | 38196 | 7: 60319670 |
| 38224 | 7: 61001713 | 38269 | 7: 61575267 | 38286 | 7: 62059589 | 38299 | 7: 62381127 | 38312 | 7: 62665355 |
| 38313 | 7: 62666818 | 38441 | 7: 65409794 | 38449 | 7: 65594792 | 38520 | 7: 66847524 | 38592 | 7: 68772935 |
| 38594 | 7: 68885530 | 38657 | 7: 70110273 | 38683 | 7: 71032462 | 38718 | 7: 71794897 | 38728 | 7: 71996424 |
| 38742 | 7: 72314529 | 38808 | 7: 74314108 | 38814 | 7: 74538065 | 38843 | 7: 75119443 | 38849 | 7: 75246858 |
| 38850 | 7: 75278782 | 38865 | 7: 75680840 | 38908 | 7: 76699078 | 38924 | 7: 77064173 | 38949 | 7: 77643339 |
| 38981 | 7: 78247892 | 38986 | 7: 78358118 | 39067 | 7: 79874457 | 39117 | 7: 80683144 | 39127 | 7: 80972557 |
| 39147 | 7: 81218151 | 39197 | 7: 82527838 | 39256 | 7: 83861078 | 39276 | 7: 84283959 | 39317 | 7: 85606256 |
| 39369 | 7: 87039982 | 39386 | 7: 87585340 | 39438 | 7: 89078282 | 39458 | 7: 89409840 | 39473 | 7: 89883975 |
| 39495 | 7: 90326242 | 39589 | 7: 91305083 | 39633 | 7: 92190820 | 39704 | 7: 94482521 | 39741 | 7: 95245462 |
| 39764 | 7: 95873877 | 39776 | 7: 96101255 | 39780 | 7: 96126223 | 39967 | 7: 96962124 | 39977 | 7: 97249811 |
| 39984 | 7: 97508045 | 39987 | 7: 97537955 | 40007 | 7: 98034786 | 40009 | 7: 98127942 | 40167 | 7: 102360026 |
| 40187 | 7: 102838629 | 40190 | 7: 102879057 | 40252 | 7: 104220122 | 40270 | 7: 104812484 | 40345 | 7: 106558358 |
| 40381 | 7: 107255212 | 40401 | 7: 108080872 | 40406 | 7: 108274048 | 40425 | 7: 108662049 | 40435 | 7: 108882531 |
| 40540 | 8: 1185446 | 40561 | 8: 1731044 | 40562 | 8: 1735452 | 40568 | 8: 1797826 | 40584 | 8: 2112938 |
| 40597 | 8: 2381761 | 40609 | 8: 2664904 | 40720 | 8: 5502050 | 40787 | 8: 7227540 | 40820 | 8: 7866886 |
| 40852 | 8: 8701133 | 40988 | 8: 11839675 | 41027 | 8: 12939122 | 41083 | 8: 14812117 | 41084 | 8: 14836734 |
| 41129 | 8: 15805194 | 41130 | 8: 15826955 | 41140 | 8: 16188640 | 41161 | 8: 16768290 | 41295 | 8: 20088784 |
| 41367 | 8: 22206603 | 41372 | 8: 22296675 | 41379 | 8: 22531070 | 41384 | 8: 22832882 | 41391 | 8: 23108057 |
| 41574 | 8: 25836147 | 41585 | 8: 26072917 | 41683 | 8: 28986201 | 41684 | 8: 29004667 | 41685 | 8: 29005955 |
| 41710 | 8: 29655591 | 41727 | 8: 29969758 | 41806 | 8: 32023600 | 41820 | 8: 32402575 | 41826 | 8: 32658867 |
| 41872 | 8: 34205302 | 41887 | 8: 34586231 | 41924 | 8: 35462325 | 41970 | 8: 36379322 | 41971 | 8: 36399226 |
| 42048 | 8: 38716745 | 42085 | 8: 39915480 | 42086 | 8: 39942972 | 42119 | 8: 40483328 | 42137 | 8: 40876799 |
| 42195 | 8: 42367753 | 42266 | 8: 44367476 | 42313 | 8: 45640500 | 42355 | 8: 46379679 | 42373 | 8: 46931988 |
| 42389 | 8: 47348455 | 42415 | 8: 47938185 | 42433 | 8: 48578484 | 42492 | 8: 50146172 | 42546 | 8: 51588079 |
| 42631 | 8: 53806641 | 42639 | 8: 54114529 | 42646 | 8: 54256907 | 42654 | 8: 54525130 | 42656 | 8: 54533690 |
| 42657 | 8: 54534313 | 42658 | 8: 54536088 | 42659 | 8: 54544973 | 42660 | 8: 54547153 | 42854 | 8: 59769666 |
| 42892 | 8: 60472436 | 42903 | 8: 60785213 | 43085 | 8: 65667278 | 43089 | 8: 65893239 | 43110 | 8: 66320758 |
| 43186 | 8: 67933084 | 43215 | 8: 68749952 | 43230 | 8: 69106626 | 43232 | 8: 69110799 | 43244 | 8: 69435453 |
| 43345 | 8: 72058951 | 43397 | 8: 73337772 | 43434 | 8: 74223661 | 43566 | 8: 77624055 | 43579 | 8: 78130709 |
| 43593 | 8: 78494713 | 43655 | 8: 80608965 | 43668 | 8: 80925878 | 43679 | 8: 81135408 | 43745 | 8: 82415599 |
| 43764 | 8: 82948559 | 43847 | 8: 85052405 | 43927 | 8: 87126956 | 43963 | 8: 87960883 | 43972 | 8: 88206973 |
| 43973 | 8: 88207717 | 44068 | 8: 90411690 | 44089 | 8: 90899216 | 44097 | 8: 91046537 | 44301 | 8: 96913052 |
| 44325 | 8: 97476895 | 44365 | 8: 98665857 | 44390 | 8: 99176725 | 44398 | 8: 99311311 | 44414 | 8: 99547619 |
| 44418 | 8: 99577548 | 44438 | 8: 100015023 | 44490 | 8: 101376581 | 44635 | 8: 103813682 | 44653 | 8: 104231786 |
| 44746 | 8: 106532357 | 44749 | 8: 106594158 | 44792 | 8: 107582608 | 45042 | 9: 2688546 | 45058 | 9: 3203477 |
| 45130 | 9: 5470319 | 45151 | 9: 6116416 | 45173 | 9: 6877480 | 45177 | 9: 7031515 | 45229 | 9: 8388451 |
| 45283 | 9: 9884501 | 45296 | 9: 10301651 | 45298 | 9: 10310168 | 45417 | 9: 11927500 | 45439 | 9: 12374207 |
| 45514 | 9: 13742514 | 45529 | 9: 14218593 | 45532 | 9: 14239370 | 45566 | 9: 15019469 | 45593 | 9: 15465522 |
| 45684 | 9: 17366049 | 45686 | 9: 17428879 | 45709 | 9: 18143920 | 45711 | 9: 18153000 | 45712 | 9: 18160118 |
| 45757 | 9: 19618032 | 45977 | 9: 25228881 | 45987 | 9: 25512136 | 46000 | 9: 25838484 | 46031 | 9: 26618713 |
| 46034 | 9: 26691579 | 46063 | 9: 27473901 | 46065 | 9: 27477523 | 46066 | 9: 27485623 | 46095 | 9: 28146296 |
| 46123 | 9: 28679350 | 46135 | 9: 28927068 | 46242 | 9: 30976439 | 46245 | 9: 31003562 | 46276 | 9: 31748693 |
| 46330 | 9: 33062497 | 46343 | 9: 33376755 | 46364 | 9: 33886935 | 46372 | 9: 34155670 | 46516 | 9: 37778578 |
| 46545 | 9: 38309457 | 46546 | 9: 38314365 | 46561 | 9: 38550987 | 46644 | 9: 40310425 | 46767 | 9: 43367400 |
| 46771 | 9: 43430747 | 46802 | 9: 44301570 | 46864 | 9: 46172575 | 46865 | 9: 46201814 | 46916 | 9: 47818319 |
| 46943 | 9: 48865487 | 46946 | 9: 48902577 | 46963 | 9: 49476706 | 46974 | 9: 49653803 | 47048 | 9: 51753467 |
| 47067 | 9: 52274853 | 47097 | 9: 52875308 | 47148 | 9: 54342081 | 47149 | 9: 54361703 | 47168 | 9: 54807613 |
| 47325 | 9: 59403628 | 47340 | 9: 59637965 | 47341 | 9: 59638253 | 47342 | 9: 59641031 | 47343 | 9: 59643481 |
| 47344 | 9: 59645062 | 47345 | 9: 59645804 | 47346 | 9: 59647105 | 47347 | 9: 59650376 | 47349 | 9: 59653721 |
| 47385 | 9: 60709648 | 47395 | 9: 60924554 | 47396 | 9: 60926410 | 47425 | 9: 61795735 | 47611 | 9: 65961632 |
| 47667 | 9: 67443259 | 47669 | 9: 67511922 | 47679 | 9: 67708264 | 47688 | 9: 68029230 | 47727 | 9: 69025344 |
| 47770 | 9: 70119558 | 47782 | 9: 70360272 | 47843 | 9: 71633299 | 47985 | 9: 75491653 | 48031 | 9: 76631634 |
| 48186 | 9: 79336612 | 48219 | 9: 80142294 | 48257 | 9: 80938431 | 48267 | 9: 81108364 | 48274 | 9: 81217242 |
| 48305 | 9: 81845883 | 48312 | 9: 82017648 | 48340 | 9: 82626166 | 48342 | 9: 82647502 | 48343 | 9: 82651491 |
| 48345 | 9: 82668654 | 48348 | 9: 82682681 | 48354 | 9: 82701768 | 48361 | 9: 82842810 | 48394 | 9: 83693006 |
| 48397 | 9: 83826419 | 48468 | 9: 85456175 | 48490 | 9: 85784712 | 48550 | 9: 87315819 | 48553 | 9: 87327271 |
| 48558 | 9: 87361936 | 48559 | 9: 87373306 | 48590 | 9: 87819557 | 48591 | 9: 87822671 | 48627 | 9: 88634584 |
| 48631 | 9: 88656403 | 48632 | 9: 88680123 | 48707 | 9: 89908123 | 48708 | 9: 89918140 | 48710 | 9: 89921487 |
| 48713 | 9: 90012380 | 48748 | 9: 90826907 | 48780 | 9: 91531504 | 48810 | 9: 92215180 | 48815 | 9: 92295269 |
| 48856 | 9: 93221725 | 48880 | 9: 93749307 | 48891 | 9: 93889254 | 48911 | 9: 94252780 | 48914 | 9: 94269583 |
| 48923 | 9: 94459307 | 48924 | 9: 94493145 | 48986 | 9: 95568256 | 49048 | 9: 96604431 | 49052 | 9: 96651231 |
| 49053 | 9: 96652474 | 49054 | 9: 96655491 | 49055 | 9: 96658405 | 49056 | 9: 96659811 | 49059 | 9: 96727179 |
| 49060 | 9: 96730432 | 49065 | 9: 96818068 | 49100 | 9: 97292214 | 49186 | 9: 99056298 | 49199 | 9: 99304134 |
| 49204 | 9: 99370466 | 49267 | 9: 100494397 | 49269 | 9: 100511637 | 49277 | 9: 100658454 | 49280 | 9: 100721863 |
| 49342 | 9: 101817009 | 49343 | 9: 101818874 | 49344 | 9: 101822513 | 49346 | 9: 101827562 | 49349 | 9: 101865213 |
| 49442 | 9: 103263715 | 49469 | 9: 103800979 | 49529 | 10: 872754 | 49531 | 10: 882204 | 49542 | 10: 1178970 |
| 49569 | 10: 1795897 | 49614 | 10: 2626851 | 49654 | 10: 3355041 | 49730 | 10: 4924755 | 49746 | 10: 5330263 |
| 49804 | 10: 6194603 | 49811 | 10: 6389416 | 49855 | 10: 7506260 | 49856 | 10: 7508517 | 49896 | 10: 8296974 |
| 49909 | 10: 8505165 | 49939 | 10: 9303914 | 49984 | 10: 10211405 | 49995 | 10: 10425877 | 50019 | 10: 10791977 |
| 50022 | 10: 10887987 | 50067 | 10: 11918946 | 50097 | 10: 12461852 | 50162 | 10: 13597734 | 50172 | 10: 13748622 |
| 50235 | 10: 15125993 | 50326 | 10: 16719617 | 50347 | 10: 17070966 | 50355 | 10: 17235899 | 50385 | 10: 17893631 |
| 50510 | 10: 20312528 | 50512 | 10: 20320283 | 50597 | 10: 21831782 | 50627 | 10: 22355891 | 50730 | 10: 26724399 |
| 50740 | 10: 26816725 | 50748 | 10: 26936267 | 50763 | 10: 27119455 | 50820 | 10: 28726853 | 50827 | 10: 28818762 |
| 50840 | 10: 28923770 | 50844 | 10: 28933854 | 50884 | 10: 29514735 | 50885 | 10: 29522253 | 50886 | 10: 29533360 |
| 50906 | 10: 30075494 | 50909 | 10: 30126897 | 50911 | 10: 30167171 | 50955 | 10: 31703939 | 50968 | 10: 32063661 |
| 50983 | 10: 32536622 | 51010 | 10: 33047373 | 51015 | 10: 33221086 | 51073 | 10: 34533777 | 51093 | 10: 34950052 |
| 51125 | 10: 35727830 | 51189 | 10: 37345559 | 51190 | 10: 37377718 | 51191 | 10: 37388003 | 51202 | 10: 37683947 |
| 51210 | 10: 37775786 | 51223 | 10: 38166114 | 51227 | 10: 38269447 | 51243 | 10: 38653361 | 51265 | 10: 39263333 |
| 51295 | 10: 40078678 | 51332 | 10: 41115727 | 51348 | 10: 41693430 | 51354 | 10: 41758465 | 51445 | 10: 44433737 |
| 51454 | 10: 44645299 | 51494 | 10: 45580212 | 51611 | 10: 47920359 | 51626 | 10: 48320530 | 51643 | 10: 48547551 |
| 51688 | 10: 49516649 | 51721 | 10: 50223805 | 51725 | 10: 50334170 | 51759 | 10: 51169084 | 51760 | 10: 51181102 |
| 51761 | 10: 51186753 | 51773 | 10: 51492328 | 51784 | 10: 51754878 | 51809 | 10: 52286224 | 51840 | 10: 52879725 |
| 51889 | 10: 54463903 | 51952 | 10: 56290718 | 51963 | 10: 56443542 | 52006 | 10: 57713193 | 52037 | 10: 58360318 |
| 52049 | 10: 58658013 | 52120 | 10: 61117665 | 52181 | 10: 62385028 | 52245 | 10: 64094738 | 52272 | 10: 64614710 |
| 52276 | 10: 64656067 | 52345 | 10: 66304541 | 52390 | 10: 67276588 | 52391 | 10: 67310241 | 52399 | 10: 67673108 |
| 52467 | 10: 68971489 | 52480 | 10: 69334027 | 52486 | 10: 69501262 | 52499 | 10: 69823517 | 52501 | 10: 69836682 |
| 52504 | 10: 69941547 | 52513 | 10: 70197389 | 52537 | 10: 71023784 | 52552 | 10: 71449748 | 52570 | 10: 72023102 |
| 52598 | 10: 73101962 | 52623 | 10: 73660537 | 52653 | 10: 74459889 | 52665 | 10: 75024558 | 52672 | 10: 75197354 |
| 52688 | 10: 75607741 | 52743 | 10: 76813636 | 52823 | 10: 78537516 | 52826 | 10: 78594348 | 52840 | 10: 79035728 |
| 52900 | 10: 80684424 | 52913 | 10: 81068664 | 52970 | 10: 81760396 | 53006 | 10: 82276845 | 53023 | 10: 82877025 |
| 53054 | 10: 83522156 | 53055 | 10: 83550757 | 53062 | 10: 83712797 | 53068 | 10: 83857093 | 53071 | 10: 83936676 |
| 53099 | 10: 84417728 | 53239 | 10: 87507691 | 53287 | 10: 88767077 | 53305 | 10: 89008507 | 53312 | 10: 89144342 |
| 53340 | 10: 89729222 | 53366 | 10: 90207696 | 53388 | 10: 90615371 | 53487 | 10: 92661034 | 53497 | 10: 92892557 |
| 53545 | 10: 94160502 | 53555 | 10: 94348103 | 53604 | 10: 95590054 | 53623 | 10: 95920911 | 53627 | 10: 96023548 |
| 53669 | 10: 97104354 | 53675 | 10: 97169425 | 53745 | 10: 98522222 | 53797 | 10: 99634106 | 53869 | 10: 101120062 |
| 53875 | 10: 101177760 | 53882 | 10: 101281677 | 53887 | 10: 101426015 | 53913 | 10: 101819543 | 53944 | 10: 102268731 |
| 54003 | 11: 441012 | 54045 | 11: 1204347 | 54069 | 11: 1549638 | 54111 | 11: 1989902 | 54174 | 11: 2689033 |
| 54209 | 11: 3235907 | 54259 | 11: 4054643 | 54292 | 11: 4984752 | 54300 | 11: 5106453 | 54327 | 11: 5677169 |
| 54409 | 11: 7670104 | 54425 | 11: 8021628 | 54476 | 11: 9219377 | 54572 | 11: 11342389 | 54630 | 11: 12715868 |
| 54638 | 11: 12870807 | 54674 | 11: 13642677 | 54677 | 11: 13704625 | 54736 | 11: 15343003 | 54745 | 11: 15626371 |
| 54747 | 11: 15658858 | 54756 | 11: 15853957 | 54797 | 11: 16775998 | 54856 | 11: 18554101 | 54872 | 11: 18844419 |
| 54875 | 11: 18948217 | 54883 | 11: 19173839 | 54923 | 11: 20562842 | 54924 | 11: 20568845 | 5498 | 11: 21832414 |
| 55037 | 11: 23157077 | 55048 | 11: 23494343 | 55084 | 11: 24320520 | 55092 | 11: 24369984 | 55093 | 11: 24377983 |
| 55094 | 11: 24393545 | 55096 | 11: 24408850 | 55119 | 11: 24944857 | 55151 | 11: 25930515 | 55161 | 11: 26154577 |
| 55174 | 11: 26362493 | 55214 | 11: 27414983 | 55238 | 11: 27959917 | 55283 | 11: 28844947 | 55302 | 11: 29230653 |
| 55315 | 11: 29502309 | 55352 | 11: 30379793 | 55380 | 11: 30989937 | 55381 | 11: 31003265 | 55382 | 11: 31020736 |
| 55383 | 11: 31023833 | 55425 | 11: 32229026 | 55458 | 11: 33116089 | 55479 | 11: 33643610 | 55496 | 11: 34099362 |
| 55532 | 11: 35548594 | 55538 | 11: 35660110 | 55568 | 11: 36429662 | 55588 | 11: 37003872 | 55608 | 11: 37326478 |
| 55615 | 11: 37508364 | 55620 | 11: 37570757 | 55624 | 11: 37639983 | 55768 | 11: 41573665 | 55824 | 11: 42850632 |
| 55835 | 11: 43167995 | 55873 | 11: 44279092 | 55880 | 11: 44503647 | 55915 | 11: 45256103 | 55925 | 11: 45835142 |
| 55933 | 11: 45970733 | 55938 | 11: 46039317 | 55941 | 11: 46045608 | 55942 | 11: 46058932 | 55972 | 11: 46675342 |
| 56017 | 11: 47326731 | 56043 | 11: 47992234 | 56067 | 11: 48522658 | 56126 | 11: 49051072 | 56190 | 11: 50710309 |
| 56222 | 11: 51848453 | 56258 | 11: 53253320 | 56273 | 11: 53826835 | 56332 | 11: 55052140 | 56335 | 11: 55137862 |
| 56337 | 11: 55149593 | 56397 | 11: 56859473 | 56653 | 11: 63292715 | 56682 | 11: 63832921 | 56683 | 11: 63837249 |
| 56714 | 11: 64511310 | 56734 | 11: 65119714 | 56739 | 11: 65250246 | 56752 | 11: 65585594 | 56764 | 11: 65826676 |
| 56767 | 11: 66038610 | 56784 | 11: 66319597 | 56812 | 11: 67082495 | 56896 | 11: 68461614 | 56913 | 11: 68937779 |
| 56917 | 11: 69060122 | 56924 | 11: 69109200 | 56925 | 11: 69118745 | 56928 | 11: 69142947 | 56933 | 11: 69181613 |
| 56943 | 11: 69335989 | 57008 | 11: 70675473 | 57082 | 11: 71401956 | 57156 | 11: 73126654 | 57200 | 11: 74123227 |
| 57271 | 11: 75902686 | 57350 | 11: 77659975 | 57451 | 11: 79225059 | 57506 | 11: 80496876 | 57543 | 11: 81586331 |
| 57554 | 11: 81811191 | 57559 | 11: 81888932 | 57573 | 11: 82234600 | 57724 | 11: 85632013 | 57806 | 11: 87517972 |
| 57868 | 11: 88067171 | 57900 | 11: 88940240 | 57915 | 11: 89270730 | 57969 | 11: 90490496 | 57973 | 11: 90559289 |
| 57995 | 11: 91106611 | 58020 | 11: 91612293 | 58045 | 11: 92156116 | 58120 | 11: 93813789 | 58133 | 11: 94097451 |
| 58153 | 11: 94563151 | 58180 | 11: 95385268 | 58195 | 11: 95727670 | 58219 | 11: 96378020 | 58238 | 11: 97095593 |
| 58246 | 11: 97197864 | 58308 | 11: 98538984 | 58364 | 11: 99473375 | 58417 | 11: 100532361 | 58536 | 11: 102829142 |
| 58591 | 11: 103534827 | 58607 | 11: 104097856 | 58720 | 11: 106485352 | 58877 | 12: 3502498 | 58943 | 12: 5186588 |
| 58945 | 12: 5325638 | 58966 | 12: 6004879 | 58987 | 12: 6773018 | 59008 | 12: 7507940 | 59043 | 12: 8830007 |
| 59057 | 12: 9272154 | 59061 | 12: 9329563 | 59069 | 12: 9522013 | 59119 | 12: 10577179 | 59120 | 12: 10585260 |
| 59155 | 12: 11315928 | 59176 | 12: 11720838 | 59179 | 12: 11790945 | 59182 | 12: 11827023 | 59184 | 12: 11857024 |
| 59208 | 12: 12366690 | 59238 | 12: 13384011 | 59260 | 12: 13823314 | 59276 | 12: 14077064 | 59282 | 12: 14150866 |
| 59301 | 12: 14486563 | 59316 | 12: 14918565 | 59397 | 12: 16476525 | 59417 | 12: 16974977 | 59428 | 12: 17253883 |
| 59434 | 12: 17359694 | 59439 | 12: 17465924 | 59454 | 12: 17933719 | 59471 | 12: 18284739 | 59549 | 12: 20109323 |
| 59698 | 12: 23940231 | 59702 | 12: 24068026 | 59727 | 12: 24577728 | 59786 | 12: 25839029 | 59799 | 12: 26457768 |
| 59840 | 12: 27377754 | 59846 | 12: 27488955 | 59870 | 12: 28122480 | 59956 | 12: 30649835 | 59989 | 12: 31264310 |
| 60008 | 12: 31780482 | 60117 | 12: 34750935 | 60139 | 12: 35299411 | 60141 | 12: 35311925 | 60142 | 12: 35312992 |
| 60143 | 12: 35314580 | 60144 | 12: 35321842 | 60145 | 12: 35326856 | 60147 | 12: 35334692 | 60148 | 12: 35339381 |
| 60149 | 12: 35342141 | 60151 | 12: 35355118 | 60152 | 12: 35355785 | 60153 | 12: 35363362 | 60154 | 12: 35424126 |
| 60156 | 12: 35487159 | 60202 | 12: 37059151 | 60206 | 12: 37121439 | 60347 | 12: 42536086 | 60352 | 12: 42838412 |
| 60389 | 12: 44505965 | 60458 | 12: 46806766 | 60555 | 12: 49288668 | 60578 | 12: 49872127 | 60583 | 12: 49903248 |
| 60596 | 12: 50116234 | 60604 | 12: 50253408 | 60730 | 12: 53098596 | 60840 | 12: 55588645 | 60862 | 12: 56409050 |
| 60875 | 12: 56736797 | 60935 | 12: 58879036 | 61043 | 12: 62502730 | 61081 | 12: 63602083 | 61084 | 12: 63713299 |
| 61163 | 12: 65908229 | 61179 | 12: 66475638 | 61325 | 12: 69535302 | 61424 | 12: 73231474 | 61483 | 12: 74215241 |
| 61488 | 12: 74344277 | 61565 | 12: 76057266 | 61574 | 12: 76294965 | 61575 | 12: 76298488 | 61597 | 12: 76881665 |
| 61771 | 12: 80709446 | 61777 | 12: 80884100 | 61814 | 12: 81734916 | 61831 | 12: 82087879 | 61844 | 12: 82441644 |
| 61856 | 12: 82591128 | 61912 | 12: 83854133 | 61961 | 12: 84933491 | 61993 | 12: 85357065 | 62129 | 13: 1206621 |
| 62198 | 13: 2453852 | 62215 | 13: 2711565 | 62223 | 13: 2835285 | 62289 | 13: 4735864 | 62347 | 13: 5936143 |
| 62362 | 13: 6285905 | 62525 | 13: 10419094 | 62677 | 13: 14935831 | 62696 | 13: 15681048 | 62771 | 13: 17258775 |
| 62815 | 13: 17832527 | 62916 | 13: 19454627 | 62947 | 13: 19907032 | 62968 | 13: 20390279 | 62969 | 13: 20405413 |
| 62970 | 13: 20411494 | 62971 | 13: 20423875 | 62972 | 13: 20430234 | 62988 | 13: 20701775 | 63002 | 13: 21060977 |
| 63055 | 13: 22024146 | 63112 | 13: 23989482 | 63141 | 13: 24644133 | 63143 | 13: 24667549 | 63150 | 13: 24857890 |
| 63174 | 13: 25338856 | 63181 | 13: 25455632 | 63193 | 13: 25724492 | 63199 | 13: 25901617 | 63240 | 13: 27082022 |
| 63282 | 13: 27909495 | 63285 | 13: 28007570 | 63295 | 13: 28335861 | 63305 | 13: 28545136 | 63341 | 13: 29355083 |
| 63359 | 13: 29815935 | 63424 | 13: 31076841 | 63427 | 13: 31114025 | 63436 | 13: 31276115 | 63470 | 13: 31684931 |
| 63484 | 13: 32188070 | 63485 | 13: 32192701 | 63486 | 13: 32195543 | 63488 | 13: 32202239 | 63490 | 13: 32214896 |
| 63507 | 13: 32509667 | 63513 | 13: 32595507 | 63515 | 13: 32606184 | 63519 | 13: 32649822 | 63520 | 13: 32651100 |
| 63644 | 13: 35586171 | 63673 | 13: 36380278 | 63730 | 13: 37696035 | 63799 | 13: 39119033 | 63842 | 13: 39974325 |
| 63843 | 13: 40001169 | 63848 | 13: 40028952 | 63884 | 13: 40680902 | 63923 | 13: 41808005 | 63934 | 13: 41965173 |
| 63957 | 13: 42527459 | 64015 | 13: 43212135 | 64028 | 13: 43604201 | 64041 | 13: 43854540 | 64042 | 13: 43855779 |
| 64043 | 13: 43868655 | 64044 | 13: 43869987 | 64045 | 13: 43870636 | 64046 | 13: 43872533 | 64047 | 13: 43873040 |
| 64048 | 13: 43873873 | 64049 | 13: 43876772 | 64050 | 13: 43878363 | 64051 | 13: 43879388 | 64052 | 13: 43880125 |
| 64053 | 13: 43884214 | 64258 | 13: 47680398 | 64280 | 13: 48210593 | 64321 | 13: 49204706 | 64383 | 13: 51177851 |
| 64397 | 13: 51341948 | 64439 | 13: 52703815 | 64510 | 13: 53977365 | 64538 | 13: 54845724 | 64978 | 13: 64716430 |
| 64982 | 13: 64796939 | 64998 | 13: 65331493 | 65047 | 13: 66193660 | 65061 | 13: 66407606 | 65093 | 13: 67181101 |
| 65096 | 13: 67321578 | 65215 | 13: 70526426 | 65221 | 13: 70575324 | 65237 | 13: 70871738 | 65271 | 13: 71637090 |
| 65328 | 13: 72957542 | 65357 | 13: 73471150 | 65381 | 13: 73812183 | 65456 | 13: 75531070 | 65529 | 13: 76798256 |
| 65547 | 13: 77244637 | 65550 | 13: 77279091 | 65578 | 13: 77845959 | 65598 | 13: 78300577 | 65660 | 13: 79330804 |
| 65677 | 13: 79542295 | 65819 | 13: 82171173 | 65917 | 14: 1204169 | 65929 | 14: 1645654 | 66049 | 14: 3651551 |
| 66072 | 14: 4085451 | 66078 | 14: 4200331 | 66116 | 14: 4961976 | 66160 | 14: 5700495 | 66209 | 14: 6316411 |
| 66268 | 14: 7449642 | 66271 | 14: 7527096 | 66317 | 14: 8213069 | 66475 | 14: 10912061 | 66523 | 14: 11744884 |
| 66538 | 14: 12080094 | 66544 | 14: 12195646 | 66565 | 14: 12541653 | 66566 | 14: 12551654 | 66568 | 14: 12570995 |
| 66627 | 14: 14043986 | 66718 | 14: 16010978 | 66719 | 14: 16022809 | 66722 | 14: 16058309 | 66732 | 14: 16280608 |
| 66746 | 14: 16568761 | 66820 | 14: 18106996 | 66821 | 14: 18115961 | 66855 | 14: 19074429 | 66858 | 14: 19172311 |
| 66877 | 14: 19623114 | 66903 | 14: 20293557 | 66912 | 14: 20629249 | 66916 | 14: 20662609 | 66917 | 14: 20673221 |
| 66925 | 14: 20792640 | 67108 | 14: 22916046 | 67140 | 14: 22986080 | 67146 | 14: 22999212 | 67302 | 14: 23338890 |
| 67351 | 14: 23565143 | 67354 | 14: 23600267 | 67355 | 14: 23607944 | 67357 | 14: 23630896 | 67359 | 14: 23652804 |
| 67369 | 14: 23826318 | 67371 | 14: 23829066 | 67374 | 14: 23831133 | 67501 | 14: 24941221 | 67613 | 14: 26283998 |
| 67660 | 14: 27607287 | 67681 | 14: 28330433 | 67709 | 14: 29096850 | 67747 | 14: 29873926 | 67772 | 14: 30389540 |
| 67822 | 14: 31570898 | 67845 | 14: 32267064 | 67901 | 14: 33278402 | 67955 | 14: 34370911 | 68032 | 14: 36313786 |
| 68053 | 14: 36889990 | 68099 | 14: 38260754 | 68128 | 14: 39143238 | 68172 | 14: 40259820 | 68190 | 14: 40976286 |
| 68197 | 14: 41229576 | 68198 | 14: 41237524 | 68261 | 14: 42746359 | 68276 | 14: 43241727 | 68313 | 14: 44566510 |
| 68321 | 14: 44666155 | 68354 | 14: 45237526 | 68360 | 14: 45367206 | 68364 | 14: 45505076 | 68368 | 14: 45610432 |
| 68441 | 14: 46995812 | 68448 | 14: 47163738 | 68460 | 14: 47428677 | 68478 | 14: 48150783 | 68509 | 14: 48767270 |
| 68536 | 14: 49487643 | 68580 | 14: 50970280 | 68581 | 14: 50986924 | 68606 | 14: 51659332 | 68620 | 14: 51985037 |
| 68654 | 14: 52815945 | 68668 | 14: 53442111 | 68687 | 14: 54022606 | 68730 | 14: 54568169 | 68772 | 14: 55536222 |
| 68826 | 14: 57247647 | 68861 | 14: 57991139 | 68870 | 14: 58182404 | 68871 | 14: 58201975 | 68907 | 14: 58964090 |
| 68974 | 14: 60591022 | 68975 | 14: 60610629 | 68988 | 14: 60975742 | 69085 | 14: 62782594 | 69115 | 14: 63450732 |
| 69130 | 14: 63928126 | 69158 | 14: 64538337 | 69184 | 14: 65420223 | 69193 | 14: 65688670 | 69200 | 14: 65782382 |
| 69231 | 14: 66405235 | 69239 | 14: 66655367 | 69251 | 14: 66916531 | 69316 | 14: 68245567 | 69466 | 14: 72276625 |
| 69482 | 14: 72695548 | 69536 | 14: 73988875 | 69639 | 14: 76386785 | 69745 | 14: 78754068 | 69764 | 14: 79462729 |
| 69867 | 14: 81514348 | 69890 | 14: 81754335 | 69891 | 14: 81755109 | 69932 | 15: 549186 | 69952 | 15: 1223740 |
| 69971 | 15: 1763083 | 70074 | 15: 4197179 | 70097 | 15: 4603953 | 70120 | 15: 5062052 | 70314 | 15: 6267325 |
| 70382 | 15: 7840165 | 70418 | 15: 8668231 | 70426 | 15: 8980586 | 70486 | 15: 10462503 | 70525 | 15: 11922022 |
| 70590 | 15: 14196834 | 70637 | 15: 15315326 | 70696 | 15: 16104588 | 70710 | 15: 16454917 | 70736 | 15: 17101878 |
| 70782 | 15: 18196851 | 70794 | 15: 18357447 | 70804 | 15: 18736931 | 70814 | 15: 19007825 | 70838 | 15: 19653453 |
| 70946 | 15: 22147187 | 70997 | 15: 23248430 | 71016 | 15: 23621459 | 71018 | 15: 23665519 | 71079 | 15: 24372603 |
| 71091 | 15: 24613968 | 71107 | 15: 24837501 | 71123 | 15: 25257089 | 71128 | 15: 25308638 | 71168 | 15: 26173379 |
| 71188 | 15: 26573138 | 71308 | 15: 28904879 | 71323 | 15: 29119476 | 71357 | 15: 30139511 | 71368 | 15: 30396261 |
| 71408 | 15: 31365259 | 71434 | 15: 31780017 | 71443 | 15: 32064162 | 71478 | 15: 32883165 | 71517 | 15: 33758501 |
| 71518 | 15: 33767591 | 71580 | 15: 34828367 | 71614 | 15: 35688175 | 71626 | 15: 35959116 | 71745 | 15: 38587541 |
| 71763 | 15: 38952840 | 71840 | 15: 40889581 | 71862 | 15: 41323112 | 71923 | 15: 42472180 | 71943 | 15: 42904887 |
| 72011 | 15: 43795583 | 72016 | 15: 43848104 | 72053 | 15: 44559517 | 72177 | 15: 46955071 | 72194 | 15: 47343068 |
| 72237 | 15: 48145511 | 72241 | 15: 48207972 | 72244 | 15: 48254047 | 72281 | 15: 49497449 | 72308 | 15: 50463550 |
| 72408 | 15: 52214824 | 72429 | 15: 52810027 | 72524 | 15: 54359875 | 72578 | 15: 55624075 | 72586 | 15: 55875613 |
| 72693 | 15: 57504930 | 72717 | 15: 57932533 | 72747 | 15: 58462557 | 72792 | 15: 59699546 | 72793 | 15: 59714205 |
| 72835 | 15: 60939376 | 72865 | 15: 61561772 | 72880 | 15: 61802396 | 72881 | 15: 61805665 | 72882 | 15: 61806975 |
| 72885 | 15: 61812433 | 72890 | 15: 61960293 | 72891 | 15: 61964427 | 72930 | 15: 62856332 | 73076 | 15: 65465351 |
| 73108 | 15: 66183419 | 73192 | 15: 68248786 | 73261 | 15: 70440283 | 73286 | 15: 71178088 | 73301 | 15: 71472435 |
| 73332 | 15: 72309114 | 73377 | 15: 73291708 | 73393 | 15: 73582243 | 73416 | 15: 74163082 | 73451 | 15: 74779756 |
| 73466 | 15: 75089743 | 73510 | 15: 76195722 | 73541 | 15: 76835072 | 73630 | 15: 78278239 | 73698 | 15: 80309655 |
| 73754 | 15: 81295713 | 73762 | 15: 81438153 | 73813 | 15: 82751747 | 73823 | 15: 82918904 | 73880 | 15: 83865721 |
| 73959 | 16: 1501024 | 74086 | 16: 4079477 | 74089 | 16: 4114086 | 74111 | 16: 4443184 | 74212 | 16: 7478039 |
| 74284 | 16: 10121160 | 74304 | 16: 10857824 | 74327 | 16: 11521662 | 74329 | 16: 11621503 | 74349 | 16: 12056975 |
| 74446 | 16: 14409239 | 74451 | 16: 14518175 | 74462 | 16: 14841844 | 74479 | 16: 15208552 | 74481 | 16: 15279430 |
| 74498 | 16: 15816016 | 74506 | 16: 16013694 | 74511 | 16: 16188270 | 74513 | 16: 16194660 | 74578 | 16: 18520385 |
| 74584 | 16: 18637360 | 74591 | 16: 18804747 | 74652 | 16: 20427119 | 74853 | 16: 25049887 | 74887 | 16: 25231259 |
| 74902 | 16: 25476855 | 74955 | 16: 26802374 | 74956 | 16: 26859375 | 74969 | 16: 27035419 | 75000 | 16: 27937410 |
| 75006 | 16: 28023322 | 75055 | 16: 29594638 | 75144 | 16: 31915038 | 75148 | 16: 31970232 | 75161 | 16: 32424717 |
| 75174 | 16: 32836939 | 75253 | 16: 34714234 | 75284 | 16: 35365207 | 75318 | 16: 36337580 | 75342 | 16: 36921696 |
| 75354 | 16: 37184848 | 75457 | 16: 39917217 | 75492 | 16: 41063333 | 75508 | 16: 41613390 | 75547 | 16: 42048065 |
| 75564 | 16: 42830906 | 75611 | 16: 44815088 | 75636 | 16: 45912914 | 75654 | 16: 46353096 | 75728 | 16: 48055006 |
| 75756 | 16: 48627494 | 75757 | 16: 48635114 | 75758 | 16: 48637200 | 75759 | 16: 48639734 | 75760 | 16: 48645579 |
| 75761 | 16: 48649527 | 75825 | 16: 50275447 | 75834 | 16: 50665850 | 75880 | 16: 51861172 | 75887 | 16: 52014418 |
| 75915 | 16: 52578534 | 75975 | 16: 54055090 | 75981 | 16: 54147078 | 76006 | 16: 54922205 | 76068 | 16: 56374651 |
| 76087 | 16: 56788303 | 76136 | 16: 58112073 | 76149 | 16: 58378141 | 76153 | 16: 58406289 | 76198 | 16: 59309602 |
| 76229 | 16: 60126861 | 76246 | 16: 60365696 | 76276 | 16: 61171484 | 76282 | 16: 61360408 | 76298 | 16: 61585266 |
| 76320 | 16: 62102938 | 76325 | 16: 62226456 | 76366 | 16: 63181951 | 76484 | 16: 65323416 | 76487 | 16: 65420109 |
| 76600 | 16: 67626229 | 76631 | 16: 68532430 | 76672 | 16: 69359404 | 76687 | 16: 69663665 | 76741 | 16: 70858584 |
| 76747 | 16: 70938743 | 76846 | 16: 73114325 | 76890 | 16: 73720962 | 76916 | 16: 74372695 | 76921 | 16: 74515053 |
| 77113 | 16: 78293826 | 77116 | 16: 78337900 | 77145 | 16: 78921036 | 77151 | 16: 78991296 | 77156 | 16: 79028746 |
| 77158 | 16: 79043302 | 77226 | 16: 79983101 | 77237 | 16: 80656680 | 77361 | 17: 3014326 | 77369 | 17: 3204260 |
| 77405 | 17: 3947723 | 77459 | 17: 4815417 | 77461 | 17: 4875306 | 77475 | 17: 5216472 | 77564 | 17: 7040082 |
| 77625 | 17: 8272941 | 77711 | 17: 10199997 | 77712 | 17: 10213106 | 78005 | 17: 17163300 | 78016 | 17: 17334505 |
| 78018 | 17: 17357791 | 78027 | 17: 17451464 | 78083 | 17: 18818415 | 78126 | 17: 19479964 | 78154 | 17: 20220411 |
| 78164 | 17: 20579995 | 78176 | 17: 21008303 | 78218 | 17: 22333330 | 78297 | 17: 25079038 | 78310 | 17: 25505355 |
| 78350 | 17: 26610845 | 78351 | 17: 26613473 | 78352 | 17: 26635840 | 78357 | 17: 26689830 | 78358 | 17: 26694252 |
| 78359 | 17: 26696213 | 78360 | 17: 26697673 | 78362 | 17: 26701612 | 78363 | 17: 26731287 | 78365 | 17: 26786597 |
| 78434 | 17: 28547562 | 78459 | 17: 29295556 | 78584 | 17: 32536552 | 7858 | 17: 32649823 | 78592 | 17: 32740716 |
| 78597 | 17: 32819809 | 78603 | 17: 32962598 | 78617 | 17: 33367107 | 78663 | 17: 34481729 | 78680 | 17: 34921588 |
| 78756 | 17: 37414365 | 78774 | 17: 38086662 | 78821 | 17: 39604254 | 78835 | 17: 39829382 | 78858 | 17: 40028314 |
| 78866 | 17: 40286179 | 78900 | 17: 40901144 | 78909 | 17: 41080474 | 78952 | 17: 42386980 | 79026 | 17: 44070161 |
| 79035 | 17: 44280518 | 79050 | 17: 44811665 | 79055 | 17: 44886429 | 79088 | 17: 45543757 | 79096 | 17: 45774161 |
| 79102 | 17: 45892895 | 79105 | 17: 45908272 | 79147 | 17: 46796152 | 79149 | 17: 46809775 | 79150 | 17: 46813078 |
| 79151 | 17: 46817568 | 79152 | 17: 46826871 | 79165 | 17: 47298668 | 79168 | 17: 47374048 | 79198 | 17: 48075712 |
| 79217 | 17: 48705395 | 79225 | 17: 48883686 | 79261 | 17: 49706199 | 79286 | 17: 50270053 | 79354 | 17: 51608281 |
| 79389 | 17: 52115500 | 79391 | 17: 52143477 | 79448 | 17: 53925709 | 7946 | 17: 54247909 | 79477 | 17: 54494775 |
| 79546 | 17: 56254970 | 79547 | 17: 56259657 | 79577 | 17: 57167393 | 79593 | 17: 57507972 | 79621 | 17: 57996907 |
| 79692 | 17: 59257237 | 79708 | 17: 59553144 | 79724 | 17: 59967182 | 79763 | 17: 60561298 | 79767 | 17: 60653296 |
| 79776 | 17: 60751860 | 79777 | 17: 60776828 | 79791 | 17: 60997432 | 79792 | 17: 61002338 | 79793 | 17: 61005235 |
| 79794 | 17: 61005949 | 79799 | 17: 61043189 | 79800 | 17: 61068599 | 79804 | 17: 61124602 | 79805 | 17: 61125930 |
| 79806 | 17: 61128578 | 79809 | 17: 61149706 | 79811 | 17: 61202835 | 79891 | 17: 63194677 | 79894 | 17: 63214725 |
| 79897 | 17: 63260940 | 79924 | 17: 63621804 | 79982 | 17: 64851883 | 80057 | 17: 66198895 | 80059 | 17: 66227047 |
| 80104 | 17: 67019380 | 80140 | 17: 68128330 | 80158 | 17: 68440573 | 80180 | 17: 68951402 | 80183 | 17: 68969488 |
| 80198 | 17: 69224867 | 80234 | 17: 70077612 | 80285 | 17: 70987613 | 80306 | 17: 71441658 | 80354 | 17: 72302909 |
| 80450 | 18: 1522517 | 80502 | 18: 1802503 | 80508 | 18: 1955689 | 80546 | 18: 2744105 | 80574 | 18: 3295457 |
| 80617 | 18: 4333966 | 80629 | 18: 4581539 | 80638 | 18: 4763164 | 80660 | 18: 5336542 | 80662 | 18: 5358167 |
| 80679 | 18: 5624880 | 80687 | 18: 5808571 | 80698 | 18: 6108723 | 80706 | 18: 6166618 | 80715 | 18: 6297029 |
| 80718 | 18: 6328373 | 80801 | 18: 7890552 | 80807 | 18: 8137616 | 80811 | 18: 8163590 | 80812 | 18: 8166965 |
| 80813 | 18: 8169505 | 80832 | 18: 8512615 | 80833 | 18: 8514080 | 80853 | 18: 8977357 | 80855 | 18: 9002587 |
| 80856 | 18: 9003665 | 80857 | 18: 9005002 | 80860 | 18: 9032209 | 80867 | 18: 9092871 | 80870 | 18: 9135683 |
| 80881 | 18: 9393342 | 80892 | 18: 9513766 | 80943 | 18: 10501656 | 80948 | 18: 10593010 | 80951 | 18: 10632938 |
| 80976 | 18: 11155672 | 81045 | 18: 12950981 | 81047 | 18: 12967212 | 81076 | 18: 13742613 | 81096 | 18: 14219190 |
| 81124 | 18: 15295023 | 81131 | 18: 15355195 | 81156 | 18: 16027392 | 81169 | 18: 16320799 | 81178 | 18: 16458903 |
| 81221 | 18: 17381122 | 81234 | 18: 17689118 | 81235 | 18: 17739758 | 81305 | 18: 19009400 | 81313 | 18: 19119273 |
| 81333 | 18: 19791460 | 81431 | 18: 21965199 | 81444 | 18: 22114555 | 81457 | 18: 22311504 | 81475 | 18: 22771950 |
| 81480 | 18: 22863942 | 81493 | 18: 23128319 | 81518 | 18: 23726377 | 81540 | 18: 24231814 | 81553 | 18: 24599361 |
| 81590 | 18: 25135273 | 81669 | 18: 26830057 | 81689 | 18: 27317835 | 81727 | 18: 28307360 | 81772 | 18: 29581458 |
| 81773 | 18: 29587945 | 81783 | 18: 29882243 | 81793 | 18: 30199537 | 81805 | 18: 30616085 | 81868 | 18: 32286982 |
| 81873 | 18: 32423923 | 81905 | 18: 33102972 | 81907 | 18: 33133073 | 81990 | 18: 35092205 | 82005 | 18: 35445465 |
| 82073 | 18: 36962507 | 82091 | 18: 37442129 | 82129 | 18: 38169007 | 82139 | 18: 38310010 | 82172 | 18: 39170079 |
| 82266 | 18: 41092657 | 82284 | 18: 41476993 | 82310 | 18: 42077536 | 82369 | 18: 43146823 | 82379 | 18: 43316323 |
| 82386 | 18: 43394501 | 82412 | 18: 43955990 | 82472 | 18: 45795414 | 82478 | 18: 45922293 | 82497 | 18: 46310174 |
| 82503 | 18: 46383891 | 82580 | 18: 46784427 | 82591 | 18: 46951310 | 82605 | 18: 47321091 | 82699 | 18: 49081014 |
| 82702 | 18: 49135986 | 82724 | 18: 49817333 | 82819 | 18: 51867088 | 82926 | 18: 54273698 | 82986 | 18: 55259160 |
| 83048 | 18: 56589786 | 83125 | 18: 57434771 | 83138 | 18: 57567593 | 83261 | 18: 60011920 | 83313 | 18: 61667250 |
| 83390 | 18: 63188823 | 83451 | 18: 64129214 | 83452 | 18: 64134704 | 83491 | 18: 64654290 | 83495 | 18: 64731877 |
| 83505 | 18: 64973481 | 83524 | 18: 65148413 | 83569 | 18: 65462851 | 83715 | 19: 4421663 | 83817 | 19: 6962233 |
| 83826 | 19: 7152680 | 83862 | 19: 7718388 | 83876 | 19: 8025749 | 83881 | 19: 8096180 | 83923 | 19: 8635132 |
| 84010 | 19: 10293568 | 84014 | 19: 10327116 | 84026 | 19: 10599040 | 84071 | 19: 11761802 | 84072 | 19: 11769174 |
| 84089 | 19: 12304263 | 84170 | 19: 13636619 | 84217 | 19: 14667736 | 84289 | 19: 16236117 | 84327 | 19: 16897668 |
| 84339 | 19: 17077928 | 84357 | 19: 17402114 | 84369 | 19: 17494959 | 84377 | 19: 17756500 | 84400 | 19: 18264868 |
| 84459 | 19: 19573243 | 84465 | 19: 19674513 | 84603 | 19: 21820203 | 84607 | 19: 21992770 | 84702 | 19: 23904908 |
| 84739 | 19: 24347524 | 84755 | 19: 24595008 | 84785 | 19: 24888212 | 84819 | 19: 25338790 | 84846 | 19: 25706233 |
| 84863 | 19: 26003355 | 85007 | 19: 28109982 | 85052 | 19: 29119243 | 85053 | 19: 29121068 | 85078 | 19: 29541741 |
| 85111 | 19: 30203261 | 85117 | 19: 30384170 | 85124 | 19: 30490687 | 85126 | 19: 30515659 | 85144 | 19: 30924489 |
| 85204 | 19: 32369220 | 85387 | 19: 35695286 | 85431 | 19: 36295776 | 85435 | 19: 36339138 | 85474 | 19: 36999184 |
| 85492 | 19: 37433519 | 85531 | 19: 38678725 | 85532 | 19: 38692719 | 85628 | 19: 40886898 | 85633 | 19: 40929680 |
| 85663 | 19: 41630709 | 85664 | 19: 41637084 | 85678 | 19: 41804462 | 85753 | 19: 43355438 | 85773 | 19: 43531840 |
| 85794 | 19: 44097163 | 85831 | 19: 44865863 | 85905 | 19: 46015589 | 85937 | 19: 47051179 | 85964 | 19: 47510334 |
| 85968 | 19: 47580297 | 85999 | 19: 48115274 | 86024 | 19: 48601037 | 86035 | 19: 48902647 | 86036 | 19: 48931565 |
| 86071 | 19: 49607062 | 86084 | 19: 49887742 | 86208 | 19: 51505417 | 86224 | 19: 51685255 | 86249 | 19: 52343433 |
| 86364 | 19: 54493744 | 86401 | 19: 55196487 | 86568 | 19: 56726108 | 86587 | 19: 57128485 | 86617 | 19: 57698375 |
| 86625 | 19: 58000641 | 86641 | 19: 58231133 | 86813 | 19: 61050416 | 86922 | 19: 62757680 | 87001 | 20: 973990 |
| 87030 | 20: 1623518 | 87122 | 20: 3090504 | 87167 | 20: 4139957 | 87275 | 20: 4685771 | 87328 | 20: 4885163 |
| 87329 | 20: 4922714 | 87330 | 20: 4927232 | 87333 | 20: 4936772 | 87428 | 20: 5478088 | 87551 | 20: 7762309 |
| 87571 | 20: 8209150 | 87587 | 20: 8561631 | 87601 | 20: 8997165 | 87640 | 20: 9653842 | 87783 | 20: 12796928 |
| 87828 | 20: 14014441 | 87898 | 20: 16397316 | 87943 | 20: 17594397 | 88015 | 20: 19691235 | 88233 | 20: 23492042 |
| 88295 | 20: 24462304 | 88299 | 20: 24489532 | 88400 | 20: 27494213 | 88428 | 20: 28090982 | 88464 | 20: 28752172 |
| 88487 | 20: 29353765 | 88516 | 20: 30159496 | 88606 | 20: 32897468 | 88712 | 20: 35378617 | 88730 | 20: 35746614 |
| 88754 | 20: 36168903 | 88765 | 20: 36410567 | 88793 | 20: 37052070 | 88823 | 20: 37587442 | 88896 | 20: 39414225 |
| 88910 | 20: 39546261 | 88982 | 20: 41144615 | 89021 | 20: 41891717 | 89030 | 20: 42067151 | 89088 | 20: 43711039 |
| 89200 | 20: 46860182 | 89213 | 20: 47186127 | 89219 | 20: 47416742 | 89252 | 20: 48508646 | 89386 | 20: 52743029 |
| 89390 | 20: 52943893 | 89391 | 20: 52951946 | 89435 | 20: 54078693 | 89459 | 20: 54797651 | 89501 | 20: 55920256 |
| 89547 | 20: 56907438 | 89580 | 20: 57704990 | 89603 | 20: 58030736 | 89614 | 20: 58246871 | 89619 | 20: 58356797 |
| 89640 | 20: 58731368 | 89642 | 20: 58744546 | 89643 | 20: 58745645 | 89644 | 20: 58746207 | 89645 | 20: 58747818 |
| 89646 | 20: 58748682 | 89658 | 20: 58932980 | 89659 | 20: 58935094 | 89663 | 20: 59004178 | 89666 | 20: 59061779 |
| 89667 | 20: 59062045 | 89674 | 20: 59260188 | 89684 | 20: 59558955 | 89695 | 20: 59880963 | 89706 | 20: 60058642 |
| 89712 | 20: 60329519 | 89766 | 20: 61949465 | 89781 | 20: 62256568 | 89802 | 20: 62677110 | 89850 | 20: 63825165 |
| 89883 | 20: 64360760 | 89988 | 20: 66882747 | 89994 | 20: 67014371 | 90009 | 20: 67434867 | 90052 | 20: 68158435 |
| 90064 | 20: 68358778 | 90067 | 20: 68381160 | 90101 | 20: 68986868 | 90122 | 20: 69449647 | 90149 | 20: 70017030 |
| 90171 | 20: 70615282 | 90299 | 21: 1225541 | 90386 | 21: 4129789 | 90390 | 21: 4195328 | 90416 | 21: 5081944 |
| 90422 | 21: 5168513 | 90434 | 21: 5505304 | 90441 | 21: 5776117 | 90445 | 21: 5799099 | 90449 | 21: 5865604 |
| 90466 | 21: 6422404 | 90500 | 21: 7408212 | 90507 | 21: 7585642 | 90558 | 21: 8258222 | 90607 | 21: 9489941 |
| 90625 | 21: 9809810 | 90673 | 21: 10967078 | 90681 | 21: 11128214 | 90691 | 21: 11412794 | 90733 | 21: 12480366 |
| 90757 | 21: 12890512 | 90783 | 21: 13556460 | 90795 | 21: 13850729 | 90814 | 21: 14283158 | 90820 | 21: 14450483 |
| 90838 | 21: 14883665 | 90844 | 21: 15011676 | 90955 | 21: 17435585 | 90977 | 21: 17918237 | 90983 | 21: 18074742 |
| 91028 | 21: 18934542 | 91038 | 21: 19207258 | 91040 | 21: 19228388 | 91042 | 21: 19270043 | 91051 | 21: 19367037 |
| 91086 | 21: 19786899 | 91094 | 21: 19865924 | 91121 | 21: 20591184 | 91130 | 21: 20646358 | 91220 | 21: 22407987 |
| 91241 | 21: 22944882 | 91282 | 21: 24017992 | 91292 | 21: 24359607 | 91349 | 21: 26122546 | 91359 | 21: 26345324 |
| 91365 | 21: 26551273 | 91366 | 21: 26568774 | 91378 | 21: 26770078 | 91419 | 21: 28090529 | 91451 | 21: 29134066 |
| 91457 | 21: 29367658 | 91458 | 21: 29377639 | 91460 | 21: 29411882 | 91490 | 21: 30232035 | 91576 | 21: 32380295 |
| 91580 | 21: 32474563 | 91616 | 21: 33229274 | 91662 | 21: 33796466 | 91708 | 21: 34496870 | 91781 | 21: 35885347 |
| 91785 | 21: 35973488 | 91802 | 21: 36413785 | 91865 | 21: 38466535 | 91868 | 21: 38504846 | 91983 | 21: 41529939 |
| 91990 | 21: 41713492 | 92010 | 21: 42131229 | 92100 | 21: 44218636 | 92101 | 21: 44266061 | 92104 | 21: 44392496 |
| 92121 | 21: 44681616 | 92122 | 21: 44686161 | 92126 | 21: 44715443 | 92130 | 21: 44791184 | 92173 | 21: 46307247 |
| 92174 | 21: 46319841 | 92198 | 21: 46784418 | 92299 | 21: 48659647 | 92354 | 21: 49876133 | 92365 | 21: 50214540 |
| 92408 | 21: 51612911 | 92464 | 21: 53191938 | 92484 | 21: 53761679 | 92513 | 21: 54536881 | 92561 | 21: 55372174 |
| 92563 | 21: 55394291 | 92584 | 21: 55724213 | 92598 | 21: 56016789 | 92610 | 21: 56129095 | 92628 | 21: 56595068 |
| 92629 | 21: 56609576 | 92630 | 21: 56612935 | 92631 | 21: 56613938 | 92632 | 21: 56629591 | 92638 | 21: 56817682 |
| 92639 | 21: 56827233 | 92640 | 21: 56835873 | 92641 | 21: 56855414 | 92725 | 21: 57245956 | 92790 | 21: 58808829 |
| 92799 | 21: 59077402 | 92811 | 21: 59261689 | 92842 | 21: 59841484 | 92856 | 21: 59994711 | 92944 | 21: 61845132 |
| 92947 | 21: 61911883 | 92965 | 21: 62176120 | 93011 | 21: 63503056 | 93031 | 21: 63983849 | 93033 | 21: 64015058 |
| 93060 | 21: 64546209 | 93125 | 21: 65454701 | 93128 | 21: 65507947 | 93165 | 21: 66297320 | 93293 | 21: 68995998 |
| 93474 | 22: 3262622 | 93623 | 22: 6202218 | 93632 | 22: 6324139 | 93633 | 22: 6332422 | 93693 | 22: 7609231 |
| 93729 | 22: 8491450 | 93766 | 22: 9678468 | 93843 | 22: 11157683 | 93862 | 22: 11729652 | 93938 | 22: 13149187 |
| 93946 | 22: 13475890 | 94015 | 22: 15394645 | 94026 | 22: 15571331 | 94029 | 22: 15621273 | 94030 | 22: 15622509 |
| 94033 | 22: 15666979 | 94043 | 22: 15998599 | 94094 | 22: 17137009 | 94106 | 22: 17528025 | 94190 | 22: 19523346 |
| 94236 | 22: 20865658 | 94239 | 22: 20892499 | 94240 | 22: 20896013 | 94242 | 22: 20900134 | 94248 | 22: 21007069 |
| 94251 | 22: 21064016 | 94261 | 22: 21320700 | 94267 | 22: 21445580 | 94279 | 22: 21651340 | 94323 | 22: 22860295 |
| 94327 | 22: 22968191 | 94361 | 22: 23904619 | 94386 | 22: 24826985 | 94425 | 22: 25962309 | 94516 | 22: 28260377 |
| 94558 | 22: 29032150 | 94569 | 22: 29394147 | 94574 | 22: 29509152 | 94575 | 22: 29521016 | 94599 | 22: 30171429 |
| 94614 | 22: 30572708 | 94689 | 22: 32709942 | 94699 | 22: 32992978 | 94735 | 22: 34025325 | 94738 | 22: 34062706 |
| 94762 | 22: 34833811 | 94770 | 22: 34980123 | 94821 | 22: 35930996 | 94832 | 22: 36212273 | 94838 | 22: 36427709 |
| 94897 | 22: 37678461 | 94930 | 22: 38553325 | 94957 | 22: 39166570 | 94961 | 22: 39195248 | 94982 | 22: 39426381 |
| 94987 | 22: 39550615 | 94998 | 22: 39688034 | 95026 | 22: 40340595 | 95073 | 22: 41528657 | 95160 | 22: 43561183 |
| 95169 | 22: 43825654 | 95180 | 22: 44046370 | 95244 | 22: 45637290 | 95266 | 22: 45983259 | 95343 | 22: 47502907 |
| 95403 | 22: 49140396 | 95406 | 22: 49163015 | 95408 | 22: 49233906 | 95450 | 22: 50360859 | 95459 | 22: 50587868 |
| 95461 | 22: 50603931 | 95479 | 22: 50938649 | 95519 | 22: 52036264 | 95533 | 22: 52343223 | 95551 | 22: 52768115 |
| 95554 | 22: 52841252 | 95575 | 22: 53087988 | 95582 | 22: 53268605 | 95650 | 22: 54557160 | 95662 | 22: 54685597 |
| 95702 | 22: 55422111 | 95717 | 22: 55662527 | 95742 | 22: 55891155 | 95813 | 22: 56887398 | 95816 | 22: 56914900 |
| 95819 | 22: 56956077 | 96032 | 23: 149568 | 96113 | 23: 2601798 | 96118 | 23: 2653886 | 96121 | 23: 2672734 |
| 96179 | 23: 3852744 | 96189 | 23: 3981656 | 96199 | 23: 4118766 | 96251 | 23: 5090449 | 96255 | 23: 5153243 |
| 96280 | 23: 5677678 | 96282 | 23: 5716762 | 96311 | 23: 6480089 | 96347 | 23: 7174045 | 96353 | 23: 7281266 |
| 96361 | 23: 7443284 | 96593 | 23: 12225865 | 96644 | 23: 13147408 | 96654 | 23: 13421583 | 96663 | 23: 13674324 |
| 96671 | 23: 13837654 | 96672 | 23: 13841783 | 96674 | 23: 13844745 | 96675 | 23: 13850106 | 96676 | 23: 13856428 |
| 96677 | 23: 13860190 | 96678 | 23: 13867299 | 96679 | 23: 13870700 | 96680 | 23: 13875916 | 96681 | 23: 13885227 |
| 96708 | 23: 14459570 | 96719 | 23: 14657847 | 96734 | 23: 14924427 | 96758 | 23: 15231293 | 96802 | 23: 15896256 |
| 96834 | 23: 16779992 | 96854 | 23: 17439199 | 96875 | 23: 17872465 | 96879 | 23: 17919886 | 96931 | 23: 19271779 |
| 96944 | 23: 19469822 | 96946 | 23: 19474356 | 96988 | 23: 20069380 | 97027 | 23: 20827882 | 97070 | 23: 21933675 |
| 97095 | 23: 22572630 | 97114 | 23: 23252435 | 97146 | 23: 23919176 | 97176 | 23: 24440913 | 97183 | 23: 24589985 |
| 97237 | 23: 25033379 | 97239 | 23: 25047940 | 97376 | 23: 27210848 | 97398 | 23: 27410296 | 97713 | 23: 31798120 |
| 97740 | 23: 32277024 | 97758 | 23: 32470121 | 97771 | 23: 32757047 | 97785 | 23: 33017631 | 97799 | 23: 33301724 |
| 97830 | 23: 34183106 | 97879 | 23: 35808678 | 97919 | 23: 36940417 | 97987 | 23: 38818865 | 97990 | 23: 38876101 |
| 98006 | 23: 39270706 | 98145 | 23: 40884084 | 98169 | 23: 41183712 | 98196 | 23: 41831694 | 98206 | 23: 42044741 |
| 98238 | 23: 42772846 | 98243 | 23: 42890423 | 98262 | 23: 43295377 | 98268 | 23: 43447515 | 98302 | 23: 44180408 |
| 98311 | 23: 44267686 | 98338 | 23: 44807132 | 98340 | 23: 44830102 | 98351 | 23: 45020916 | 98363 | 23: 45143240 |
| 98365 | 23: 45154456 | 98366 | 23: 45154585 | 98367 | 23: 45154695 | 98369 | 23: 45170649 | 98370 | 23: 45174122 |
| 98371 | 23: 45194932 | 98372 | 23: 45209920 | 98415 | 23: 46222052 | 98487 | 23: 47700808 | 98491 | 23: 47790490 |
| 98613 | 23: 49822048 | 98646 | 23: 50347036 | 98647 | 23: 50349795 | 98648 | 23: 50361478 | 98695 | 23: 51211166 |
| 98758 | 23: 52291892 | 98780 | 24: 465578 | 98795 | 24: 844195 | 98914 | 24: 3199425 | 99033 | 24: 6106333 |
| 99089 | 24: 7522593 | 99139 | 24: 8885668 | 99310 | 24: 11057073 | 99312 | 24: 11077021 | 99344 | 24: 11884545 |
| 99368 | 24: 12540583 | 99376 | 24: 12720792 | 99407 | 24: 13477647 | 99436 | 24: 14392789 | 99449 | 24: 14713059 |
| 99481 | 24: 15784201 | 99482 | 24: 15793449 | 99520 | 24: 16988712 | 99542 | 24: 17540200 | 99608 | 24: 19628750 |
| 99633 | 24: 20394869 | 99814 | 24: 23537983 | 99838 | 24: 24399016 | 99917 | 24: 26088773 | 99969 | 24: 27563741 |
| 100022 | 24: 28948556 | 100051 | 24: 29641974 | 100053 | 24: 29754026 | 100083 | 24: 30348689 | 100125 | 24: 31468489 |
| 100149 | 24: 31977640 | 100204 | 24: 33038149 | 100247 | 24: 34170378 | 100262 | 24: 34562426 | 100316 | 24: 35736460 |
| 100427 | 24: 39087533 | 100457 | 24: 39886402 | 100506 | 24: 40530911 | 100517 | 24: 40707643 | 100546 | 24: 41182679 |
| 100560 | 24: 41739524 | 100603 | 24: 42667191 | 100619 | 24: 42900584 | 100623 | 24: 43047337 | 100646 | 24: 44068866 |
| 100667 | 24: 44457109 | 100702 | 24: 45155232 | 100708 | 24: 45303639 | 100817 | 24: 46988217 | 100842 | 24: 47695031 |
| 100848 | 24: 47858503 | 100861 | 24: 48130487 | 100927 | 24: 49987049 | 100960 | 24: 50907651 | 100967 | 24: 51121771 |
| 100974 | 24: 51293921 | 101015 | 24: 52055045 | 101040 | 24: 52603746 | 101042 | 24: 52612789 | 101103 | 24: 54184345 |
| 101132 | 24: 54726088 | 101133 | 24: 54737023 | 101135 | 24: 54745793 | 101174 | 24: 55654866 | 101209 | 24: 56364015 |
| 101214 | 24: 56468941 | 101217 | 24: 56488437 | 101221 | 24: 56542836 | 101264 | 24: 57462922 | 101329 | 24: 58647028 |
| 101359 | 24: 59350759 | 101409 | 24: 60371452 | 101438 | 24: 60938433 | 101480 | 24: 61694231 | 101482 | 24: 61715423 |
| 101486 | 24: 61776900 | 101548 | 25: 1123694 | 101554 | 25: 1211629 | 101568 | 25: 1368623 | 101741 | 25: 2890918 |
| 101757 | 25: 3291302 | 101761 | 25: 3373790 | 101762 | 25: 3388313 | 101779 | 25: 3563948 | 101807 | 25: 4175576 |
| 101833 | 25: 4621429 | 101909 | 25: 5758755 | 101916 | 25: 5934892 | 101922 | 25: 6041488 | 101970 | 25: 7127243 |
| 102009 | 25: 7910528 | 102017 | 25: 8111580 | 102058 | 25: 9294790 | 102089 | 25: 9965727 | 102096 | 25: 10068090 |
| 102163 | 25: 11303404 | 102165 | 25: 11364705 | 102181 | 25: 11635729 | 102182 | 25: 11657253 | 102214 | 25: 12395830 |
| 102217 | 25: 12420213 | 102227 | 25: 12757945 | 102253 | 25: 13410180 | 102261 | 25: 13589337 | 102275 | 25: 14025698 |
| 102289 | 25: 14337887 | 102372 | 25: 16006571 | 102395 | 25: 16397106 | 102396 | 25: 16427094 | 102524 | 25: 19554407 |
| 102537 | 25: 19935329 | 102562 | 25: 20762754 | 102600 | 25: 21483496 | 102602 | 25: 21510337 | 102641 | 25: 22365508 |
| 102653 | 25: 22668990 | 102699 | 25: 23839896 | 102714 | 25: 24079936 | 102736 | 25: 24542279 | 102742 | 25: 24617636 |
| 102767 | 25: 25119747 | 102769 | 25: 25147993 | 102791 | 25: 25378579 | 102816 | 25: 26015769 | 102932 | 25: 28026271 |
| 102975 | 25: 29198572 | 102994 | 25: 29627243 | 103031 | 25: 30198863 | 103080 | 25: 31339867 | 103118 | 25: 32018635 |
| 103199 | 25: 33663856 | 103229 | 25: 34356818 | 103305 | 25: 35625080 | 103338 | 25: 36046363 | 103342 | 25: 36137423 |
| 103378 | 25: 36442406 | 103457 | 25: 37592711 | 103460 | 25: 37631841 | 103479 | 25: 38076102 | 103536 | 25: 39097677 |
| 103596 | 25: 40251086 | 103616 | 25: 40630329 | 103683 | 25: 41733621 | 103706 | 26: 19998 | 103766 | 26: 2270743 |
| 103818 | 26: 3188174 | 103819 | 26: 3191375 | 103829 | 26: 3361204 | 103909 | 26: 5551721 | 104006 | 26: 8275246 |
| 104020 | 26: 8585213 | 104105 | 26: 10587007 | 104135 | 26: 11460715 | 104155 | 26: 11751995 | 104195 | 26: 13034814 |
| 104284 | 26: 14982249 | 104298 | 26: 15453105 | 104410 | 26: 18118254 | 104413 | 26: 18261377 | 104460 | 26: 19187481 |
| 104469 | 26: 19395441 | 104471 | 26: 19411437 | 104493 | 26: 19971593 | 104520 | 26: 20855927 | 104538 | 26: 21270336 |
| 104539 | 26: 21271111 | 104540 | 26: 21272246 | 104542 | 26: 21276672 | 104554 | 26: 21609152 | 104556 | 26: 21638374 |
| 104557 | 26: 21659298 | 104575 | 26: 22193698 | 104576 | 26: 22197941 | 104651 | 26: 24350779 | 104659 | 26: 24607752 |
| 104686 | 26: 24921440 | 104693 | 26: 25131752 | 104762 | 26: 26169541 | 104768 | 26: 26273875 | 104774 | 26: 26426669 |
| 104788 | 26: 26770718 | 104860 | 26: 28504622 | 104867 | 26: 28761933 | 104935 | 26: 30753391 | 104952 | 26: 31231421 |
| 104953 | 26: 31240168 | 105002 | 26: 32486347 | 105010 | 26: 32645140 | 105037 | 26: 33152552 | 105049 | 26: 33514228 |
| 105055 | 26: 33591046 | 105060 | 26: 33741604 | 105081 | 26: 34086479 | 105174 | 26: 36209430 | 105228 | 26: 37411119 |
| 105255 | 26: 38327739 | 105259 | 26: 38422867 | 105266 | 26: 38582767 | 105287 | 26: 39039410 | 105319 | 26: 39645163 |
| 105321 | 26: 39706475 | 105436 | 26: 42682884 | 105480 | 26: 43924743 | 105484 | 26: 44008043 | 105557 | 26: 45531818 |
| 105583 | 26: 46019051 | 105611 | 26: 46544547 | 105633 | 26: 47031869 | 105741 | 26: 49412031 | 105763 | 26: 49860726 |
| 105823 | 26: 51197832 | 105827 | 26: 51273644 | 105828 | 26: 51339616 | 105832 | 26: 51408594 | 105941 | 27: 2266510 |
| 105949 | 27: 2424175 | 106008 | 27: 3926602 | 106025 | 27: 4205889 | 106087 | 27: 5556009 | 106166 | 27: 7956657 |
| 106176 | 27: 8174752 | 106200 | 27: 8748506 | 106216 | 27: 9098489 | 106217 | 27: 9098570 | 106218 | 27: 9098663 |
| 106219 | 27: 9099087 | 106220 | 27: 9099279 | 106221 | 27: 9099292 | 106222 | 27: 9099380 | 106223 | 27: 9099465 |
| 106224 | 27: 9099696 | 106225 | 27: 9099832 | 106226 | 27: 9104046 | 106228 | 27: 9106497 | 106230 | 27: 9110724 |
| 106231 | 27: 9116774 | 106232 | 27: 9117417 | 106233 | 27: 9117873 | 106234 | 27: 9117874 | 106235 | 27: 9118114 |
| 106236 | 27: 9118136 | 106237 | 27: 9118300 | 106240 | 27: 9149222 | 106241 | 27: 9151257 | 106256 | 27: 9578023 |
| 106335 | 27: 12004591 | 106383 | 27: 13309317 | 106461 | 27: 14313738 | 106474 | 27: 14529862 | 106519 | 27: 15481603 |
| 106554 | 27: 16106255 | 106656 | 27: 18041339 | 106668 | 27: 18444933 | 106677 | 27: 18618418 | 106687 | 27: 18831735 |
| 106688 | 27: 18844548 | 106691 | 27: 18895158 | 106692 | 27: 18916416 | 106693 | 27: 18921032 | 106756 | 27: 20229425 |
| 106790 | 27: 21326986 | 106801 | 27: 21699196 | 106805 | 27: 21744178 | 106817 | 27: 21948844 | 106848 | 27: 22823774 |
| 106884 | 27: 23610622 | 106908 | 27: 24137880 | 107014 | 27: 26432887 | 107082 | 27: 27644914 | 107095 | 27: 27964245 |
| 107202 | 27: 30418455 | 107334 | 27: 32927582 | 107343 | 27: 33025196 | 107408 | 27: 34899643 | 107448 | 27: 35233200 |
| 107456 | 27: 35414868 | 107653 | 27: 39221884 | 107712 | 27: 40883338 | 107746 | 27: 41747888 | 107759 | 27: 42126889 |
| 107801 | 27: 43079592 | 107803 | 27: 43088850 | 107843 | 27: 43939855 | 107846 | 27: 43979622 | 107847 | 27: 43983990 |
| 107854 | 27: 44107624 | 107862 | 27: 44229578 | 107939 | 28: 1012326 | 107958 | 28: 1304486 | 107974 | 28: 1564870 |
| 108000 | 28: 2124177 | 108040 | 28: 3650698 | 108041 | 28: 3666554 | 108059 | 28: 4687863 | 108061 | 28: 4710151 |
| 108084 | 28: 5241897 | 108148 | 28: 6765529 | 108153 | 28: 6864730 | 108232 | 28: 8673885 | 108245 | 28: 9022551 |
| 108246 | 28: 9025349 | 108267 | 28: 9565874 | 108279 | 28: 9801762 | 108289 | 28: 9992581 | 108310 | 28: 10520846 |
| 108311 | 28: 10565818 | 108389 | 28: 12680409 | 108396 | 28: 12751017 | 108476 | 28: 14911107 | 108581 | 28: 17140257 |
| 108632 | 28: 18044696 | 108660 | 28: 18836541 | 108739 | 28: 20933740 | 108741 | 28: 20941251 | 108783 | 28: 22351391 |
| 108826 | 28: 23326980 | 108859 | 28: 24154609 | 108866 | 28: 24223951 | 108903 | 28: 25048959 | 108912 | 28: 25216858 |
| 109001 | 28: 26844276 | 109007 | 28: 27009355 | 109043 | 28: 27784145 | 109052 | 28: 27909599 | 109054 | 28: 27910886 |
| 109073 | 28: 28015394 | 109215 | 28: 31286284 | 109245 | 28: 32003701 | 109325 | 28: 33230710 | 109348 | 28: 33703563 |
| 109406 | 28: 34803927 | 109413 | 28: 34924460 | 109417 | 28: 34965171 | 109472 | 28: 36153178 | 109522 | 28: 37465397 |
| 109570 | 28: 38512629 | 109579 | 28: 38669301 | 109622 | 28: 39347343 | 109640 | 28: 39934529 | 109726 | 28: 40743505 |
| 109728 | 28: 40802700 | 109729 | 28: 40815314 | 109735 | 28: 40867971 | 109741 | 28: 40986070 | 109745 | 28: 41069948 |
| 109774 | 28: 41574166 | 109825 | 28: 42451978 | 109871 | 28: 43255478 | 109915 | 28: 44119755 | 109971 | 28: 45223304 |
| 110037 | 29: 1129729 | 110066 | 29: 1630821 | 110088 | 29: 2215375 | 110115 | 29: 2538458 | 110120 | 29: 2640739 |
| 110159 | 29: 3485853 | 110162 | 29: 3535737 | 110211 | 29: 4767448 | 110325 | 29: 8136563 | 110346 | 29: 8758233 |
| 110486 | 29: 11888737 | 110531 | 29: 12717025 | 110539 | 29: 12922913 | 110611 | 29: 14988640 | 110619 | 29: 15140865 |
| 110686 | 29: 16917842 | 110688 | 29: 16949756 | 110701 | 29: 17144041 | 110705 | 29: 17304932 | 110731 | 29: 18241209 |
| 110749 | 29: 19246346 | 110842 | 29: 21593668 | 110893 | 29: 23085229 | 110896 | 29: 23104998 | 110900 | 29: 23212423 |
| 110941 | 29: 24050623 | 111030 | 29: 26373975 | 111053 | 29: 27005732 | 111066 | 29: 27532899 | 111074 | 29: 27658327 |
| 111146 | 29: 29021609 | 111151 | 29: 29269330 | 111156 | 29: 29356340 | 111204 | 29: 29930474 | 111235 | 29: 30673349 |
| 111259 | 29: 31276001 | 111275 | 29: 31521382 | 111281 | 29: 31682726 | 111359 | 29: 33581781 | 111361 | 29: 33604607 |
| 111377 | 29: 33936893 | 111379 | 29: 33963898 | 111395 | 29: 34390629 | 111404 | 29: 34569090 | 111415 | 29: 34694545 |
| 111459 | 29: 35661091 | 111465 | 29: 35722342 | 111509 | 29: 36453498 | 111527 | 29: 36872914 | 111613 | 29: 39811611 |
| 111614 | 29: 39822689 | 111712 | 29: 41607581 | 111724 | 29: 41814129 | 111745 | 29: 42165191 | 111770 | 29: 42669048 |
| 111788 | 29: 43153261 | 111839 | 29: 43881785 | 111856 | 29: 44096512 | 111873 | 29: 44366521 | 111948 | 29: 46176719 |
| 111954 | 29: 46281096 | 112022 | 29: 47700163 | 112024 | 29: 47731596 | 112148 | 29: 50449063; | | |

and
the sites of the fifth marker panel are shown in the following table:

| NO. | Position | NO. | Position | NO. | Position | NO. | Position | NO. | Position |
|---|---|---|---|---|---|---|---|---|---|
| 36 | 1: 1247889 | 37 | 1: 1248205 | 38 | 1: 1248699 | 39 | 1: 1252437 | 46 | 1: 1417011 |
| 47 | 1: 1438871 | 48 | 1: 1439812 | 51 | 1: 1473953 | 52 | 1: 1474233 | 53 | 1: 1505639 |
| 54 | 1: 1517594 | 55 | 1: 1518088 | 56 | 1: 1523007 | 57 | 1: 1524793 | 58 | 1: 1540344 |
| 59 | 1: 1540422 | 60 | 1: 1540458 | 61 | 1: 1540555 | 119 | 1: 2769164 | 139 | 1: 3245141 |
| 144 | 1: 3303269 | 169 | 1: 3679697 | 170 | 1: 3679721 | 171 | 1: 3687775 | 185 | 1: 3908027 |
| 207 | 1: 4250046 | 209 | 1: 4276065 | 400 | 1: 8645661 | 446 | 1: 9625272 | 607 | 1: 13657759 |
| 715 | 1: 16486738 | 725 | 1: 16754495 | 738 | 1: 17263605 | 750 | 1: 17537664 | 784 | 1: 18789066 |
| 935 | 1: 23001427 | 965 | 1: 23946108 | 991 | 1: 24758865 | 993 | 1: 24782983 | 1041 | 1: 26105452 |
| 1093 | 1: 27491647 | 1117 | 1: 28566525 | 1118 | 1: 28612680 | 1152 | 1: 29520198 | 1205 | 1: 31082779 |
| 1284 | 1: 33805826 | 1294 | 1: 33964232 | 1471 | 1: 38630217 | 1603 | 1: 42156367 | 1607 | 1: 42205645 |
| 1646 | 1: 43258942 | 1650 | 1: 43390633 | 1777 | 1: 46016435 | 1879 | 1: 49422581 | 1953 | 1: 51785197 |
| 1964 | 1: 52229363 | 1967 | 1: 52281229 | 2171 | 1: 57147119 | 2172 | 1: 57180246 | 2296 | 1: 60247154 |
| 2297 | 1: 60275851 | 2332 | 1: 61100002 | 2339 | 1: 61219155 | 2362 | 1: 61796684 | 2462 | 1: 64193131 |
| 2469 | 1: 64272382 | 2530 | 1: 65583709 | 2532 | 1: 65625274 | 2535 | 1: 65763047 | 2669 | 1: 69022641 |
| 2694 | 1: 69479795 | 2715 | 1: 69942383 | 2774 | 1: 70823693 | 2916 | 1: 74460838 | 3081 | 1: 78554220 |
| 3121 | 1: 79513248 | 3351 | 1: 85398326 | 3352 | 1: 85456698 | 3354 | 1: 85501954 | 3382 | 1: 86229426 |
| 3387 | 1: 86366405 | 3411 | 1: 87047388 | 3436 | 1: 87762016 | 3496 | 1: 89623272 | 3548 | 1: 90545947 |
| 3594 | 1: 91706588 | 3654 | 1: 93372952 | 3656 | 1: 93477242 | 3826 | 1: 97778617 | 3827 | 1: 97802384 |
| 3832 | 1: 97878133 | 3860 | 1: 98721777 | 3895 | 1: 99586706 | 4190 | 1: 108556096 | 4194 | 1: 108577099 |
| 4350 | 1: 112307236 | 4383 | 1: 112837296 | 4428 | 1: 113860732 | 4434 | 1: 114102926 | 4466 | 1: 114828912 |
| 4467 | 1: 114837165 | 4530 | 1: 116786787 | 4560 | 1: 117406892 | 4601 | 1: 118129247 | 4619 | 1: 118286166 |
| 4638 | 1: 118603687 | 4640 | 1: 118638525 | 4644 | 1: 118785100 | 4645 | 1: 118808555 | 4676 | 1: 119387495 |
| 4706 | 1: 120231880 | 4757 | 1: 121536001 | 4852 | 1: 124087395 | 4901 | 1: 125202968 | 4921 | 1: 125609019 |
| 4991 | 1: 127321191 | 5033 | 1: 128301452 | 5034 | 1: 128336208 | 5132 | 1: 130745923 | 5143 | 1: 131052225 |
| 5212 | 1: 131617330 | 5252 | 1: 132852921 | 5255 | 1: 132924609 | 5257 | 1: 132963363 | 5326 | 1: 134972795 |
| 5386 | 1: 135785234 | 5387 | 1: 135785949 | 5388 | 1: 135787843 | 5389 | 1: 135788916 | 5390 | 1: 135789433 |
| 5391 | 1: 135791112 | 5448 | 1: 137501805 | 5548 | 1: 140642688 | 5607 | 1: 141638716 | 5670 | 1: 142753326 |
| 5736 | 1: 143879207 | 5833 | 1: 145815334 | 5857 | 1: 146000862 | 5863 | 1: 146077457 | 5868 | 1: 146242466 |
| 5876 | 1: 146437585 | 5882 | 1: 146612774 | 5891 | 1: 146975346 | 5965 | 1: 148149815 | 6115 | 1: 150888684 |
| 6274 | 1: 153150228 | 6320 | 1: 153989230 | 6340 | 1: 154314444 | 6342 | 1: 154371107 | 6343 | 1: 154385540 |
| 6547 | 2: 682101 | 6574 | 2: 1161894 | 6626 | 2: 2113211 | 6636 | 2: 2405667 | 6699 | 2: 4200142 |
| 6781 | 2: 5668859 | 6810 | 2: 6248437 | 6811 | 2: 6253061 | 6813 | 2: 6269804 | 6815 | 2: 6275413 |
| 6834 | 2: 6717203 | 6835 | 2: 6717548 | 6836 | 2: 6732409 | 6837 | 2: 6732955 | 6873 | 2: 7379901 |
| 6888 | 2: 7706163 | 6911 | 2: 8334857 | 6932 | 2: 8969249 | 7019 | 2: 11681348 | 7024 | 2: 11775775 |
| 7049 | 2: 12802862 | 7070 | 2: 13346155 | 7119 | 2: 14632486 | 7120 | 2: 14676143 | 7125 | 2: 14743753 |
| 7148 | 2: 15330545 | 7152 | 2: 15479415 | 7159 | 2: 15620578 | 7175 | 2: 15995516 | 7188 | 2: 16261766 |
| 7244 | 2: 17777834 | 7245 | 2: 17826748 | 7280 | 2: 18272965 | 7294 | 2: 18380242 | 7300 | 2: 18552906 |
| 7374 | 2: 20073035 | 7420 | 2: 21391926 | 7484 | 2: 23026213 | 7579 | 2: 24732559 | 7750 | 2: 28419342 |
| 7751 | 2: 28419366 | 7752 | 2: 28421072 | 8028 | 2: 35888381 | 8230 | 2: 41375205 | 8293 | 2: 42660415 |
| 8319 | 2: 43375192 | 8449 | 2: 46777670 | 8450 | 2: 46782605 | 8452 | 2: 46797429 | 8466 | 2: 47070734 |
| 8467 | 2: 47100164 | 8549 | 2: 49214428 | 8560 | 2: 49760042 | 8561 | 2: 49760735 | 8735 | 2: 54491736 |
| 8791 | 2: 55960682 | 8792 | 2: 55979085 | 9079 | 2: 63568686 | 9094 | 2: 64064953 | 9161 | 2: 65602756 |
| 9288 | 2: 69830690 | 9347 | 2: 71285767 | 9373 | 2: 72193932 | 9374 | 2: 72216636 | 9516 | 2: 76002159 |
| 9530 | 2: 76376460 | 9555 | 2: 76976290 | 9786 | 2: 83877880 | 9791 | 2: 84089700 | 10121 | 2: 91931085 |
| 10139 | 2: 92377009 | 10180 | 2: 93321855 | 10182 | 2: 93326125 | 10183 | 2: 93326273 | 10184 | 2: 93327304 |
| 10185 | 2: 93353835 | 10186 | 2: 93353877 | 10187 | 2: 93354446 | 10188 | 2: 93354619 | 10189 | 2: 93354804 |
| 10190 | 2: 93355262 | 10191 | 2: 93355271 | 10192 | 2: 93356138 | 10193 | 2: 93356208 | 10194 | 2: 93356223 |
| 10195 | 2: 93366311 | 10248 | 2: 94636270 | 10286 | 2: 95449694 | 10305 | 2: 95979209 | 10312 | 2: 96139223 |
| 10401 | 2: 98337087 | 10409 | 2: 98421675 | 10413 | 2: 98461459 | 10417 | 2: 98536760 | 10466 | 2: 99940359 |
| 10605 | 2: 103776260 | 10655 | 2: 105009833 | 10686 | 2: 105635824 | 10694 | 2: 105828123 | 10712 | 2: 106230731 |
| 10781 | 2: 107728454 | 10782 | 2: 107728517 | 10783 | 2: 107729520 | 10784 | 2: 107730636 | 10785 | 2: 107735095 |
| 10786 | 2: 107735449 | 10787 | 2: 107737533 | 10789 | 2: 107742839 | 10800 | 2: 107971542 | 10846 | 2: 109019023 |
| 10929 | 2: 111354781 | 10934 | 2: 111635526 | 10954 | 2: 112098945 | 10956 | 2: 112105210 | 10975 | 2: 112632558 |
| 10978 | 2: 112654694 | 11038 | 2: 113914830 | 11099 | 2: 115533946 | 11303 | 2: 120430113 | 11405 | 2: 123285701 |
| 11444 | 2: 124279927 | 11460 | 2: 124841893 | 11684 | 2: 128718214 | 11813 | 2: 131557054 | 11822 | 2: 131862099 |
| 11852 | 2: 132351992 | 11940 | 2: 133852858 | 11943 | 2: 133878448 | 11959 | 2: 134115936 | 11960 | 2: 134172533 |
| 12004 | 2: 134750704 | 12059 | 2: 135525484 | 12143 | 3: 950659 | 12145 | 3: 954886 | 12187 | 3: 1728119 |
| 12274 | 3: 3596795 | 12343 | 3: 4727841 | 12452 | 3: 6832814 | 12454 | 3: 6841596 | 12456 | 3: 6848201 |
| 12457 | 3: 6866175 | 12458 | 3: 6867557 | 12459 | 3: 6867867 | 12460 | 3: 6885163 | 12533 | 3: 8025174 |
| 12734 | 3: 11445314 | 12790 | 3: 12934994 | 12856 | 3: 14226434 | 12920 | 3: 15616501 | 12948 | 3: 15994930 |
| 13048 | 3: 18771436 | 13049 | 3: 18775018 | 13138 | 3: 21163145 | 13156 | 3: 21764453 | 13190 | 3: 22332383 |
| 13191 | 3: 22334033 | 13192 | 3: 22339164 | 13193 | 3: 22340631 | 13194 | 3: 22343298 | 13357 | 3: 26160439 |
| 13470 | 3: 27968845 | 13475 | 3: 28047336 | 13527 | 3: 29246450 | 13584 | 3: 30235042 | 13600 | 3: 30605190 |
| 13623 | 3: 31149250 | 13660 | 3: 31948925 | 13662 | 3: 31976577 | 13736 | 3: 32970665 | 13765 | 3: 33258971 |
| 14049 | 3: 40032182 | 14051 | 3: 40065534 | 14053 | 3: 40120645 | 14188 | 3: 43541601 | 14331 | 3: 47791771 |
| 14354 | 3: 48191758 | 14355 | 3: 48234094 | 14392 | 3: 49000989 | 14416 | 3: 49459128 | 14422 | 3: 49526251 |
| 14428 | 3: 49574985 | 14483 | 3: 49988687 | 14504 | 3: 50656944 | 14581 | 3: 52783346 | 14661 | 3: 55239885 |
| 14665 | 3: 55330131 | 14835 | 3: 60375726 | 14845 | 3: 60575455 | 14850 | 3: 60636353 | 14868 | 3: 60938213 |
| 14958 | 3: 63728694 | 14960 | 3: 63776877 | 14965 | 3: 63861865 | 14966 | 3: 63887795 | 14968 | 3: 63950495 |
| 14969 | 3: 63955495 | 14970 | 3: 63976119 | 14973 | 3: 64020621 | 14975 | 3: 64058308 | 14983 | 3: 64239387 |
| 14988 | 3: 64525583 | 14992 | 3: 64697961 | 15033 | 3: 65670943 | 15044 | 3: 66042478 | 15049 | 3: 66148244 |
| 15107 | 3: 67465318 | 15130 | 3: 67947626 | 15131 | 3: 67978806 | 15150 | 3: 68443615 | 15175 | 3: 69034450 |
| 15230 | 3: 70652398 | 15234 | 3: 70860935 | 15243 | 3: 71230722 | 15285 | 3: 72930619 | 15393 | 3: 76153357 |
| 15442 | 3: 77599781 | 15443 | 3: 77635657 | 15446 | 3: 77784239 | 15495 | 3: 79128870 | 15498 | 3: 79224313 |
| 15538 | 3: 80529583 | 15563 | 3: 81048556 | 15605 | 3: 82166845 | 15625 | 3: 82581418 | 15684 | 3: 84280062 |
| 15939 | 3: 89312804 | 15941 | 3: 89315922 | 16034 | 3: 91258808 | 16253 | 3: 96974610 | 16259 | 3: 97049079 |
| 16287 | 3: 97630051 | 16414 | 3: 100563623 | 16430 | 3: 100751713 | 16431 | 3: 100782663 | 16443 | 3: 101112794 |
| 16446 | 3: 101150016 | 16537 | 3: 102883747 | 16541 | 3: 102931082 | 16598 | 3: 104333611 | 16602 | 3: 104484207 |
| 16605 | 3: 104506752 | 16607 | 3: 104541890 | 16609 | 3: 104556518 | 16628 | 3: 104869949 | 16646 | 3: 105209686 |
| 16654 | 3: 105292781 | 16714 | 3: 105965567 | 16715 | 3: 105973059 | 16718 | 3: 106026621 | 16719 | 3: 106042336 |
| 16740 | 3: 106492826 | 16750 | 3: 106599058 | 16901 | 3: 109689309 | 17056 | 3: 113011961 | 17068 | 3: 113301217 |
| 17115 | 3: 113990093 | 17186 | 3: 114897383 | 17380 | 3: 117882488 | 17384 | 3: 117909372 | 17401 | 3: 118223118 |
| 17402 | 3: 118253078 | 17639 | 4: 4003154 | 17644 | 4: 4149740 | 17697 | 4: 5004514 | 17705 | 4: 5165078 |
| 17706 | 4: 5173632 | 17740 | 4: 5747787 | 17752 | 4: 6130688 | 17761 | 4: 6330228 | 17968 | 4: 10578187 |
| 17969 | 4: 10580814 | 18198 | 4: 16670558 | 18273 | 4: 18595000 | 18276 | 4: 18619515 | 18318 | 4: 19200647 |
| 18378 | 4: 20303118 | 18635 | 4: 26901086 | 18636 | 4: 26934291 | 18820 | 4: 32010653 | 18843 | 4: 32768824 |
| 18949 | 4: 35971843 | 19100 | 4: 40158341 | 19134 | 4: 40946237 | 19168 | 4: 41815635 | 19173 | 4: 42048079 |
| 19208 | 4: 43120197 | 19214 | 4: 43203876 | 19250 | 4: 44121669 | 19285 | 4: 45161683 | 19356 | 4: 47190294 |
| 19377 | 4: 47463756 | 19400 | 4: 48100380 | 19424 | 4: 48952233 | 19466 | 4: 49461342 | 19477 | 4: 49813182 |
| 19478 | 4: 49842196 | 19480 | 4: 49888592 | 19866 | 4: 61781138 | 19888 | 4: 62276469 | 19891 | 4: 62357256 |
| 19931 | 4: 63512286 | 19965 | 4: 64527362 | 19982 | 4: 64979970 | 20028 | 4: 66103436 | 20075 | 4: 67374218 |
| 20136 | 4: 69236841 | 20289 | 4: 73424213 | 20513 | 4: 78984643 | 20553 | 4: 80105286 | 20554 | 4: 80107022 |
| 20555 | 4: 80107183 | 20556 | 4: 80107305 | 20557 | 4: 80107320 | 20558 | 4: 80107386 | 20559 | 4: 80107434 |
| 20560 | 4: 80107694 | 20561 | 4: 80107717 | 20562 | 4: 80107759 | 20674 | 4: 82773442 | 20681 | 4: 82863329 |
| 20749 | 4: 85034221 | 20779 | 4: 85832979 | 20780 | 4: 85866052 | 20788 | 4: 85968886 | 20834 | 4: 87033808 |
| 20882 | 4: 88417629 | 20886 | 4: 88515168 | 20903 | 4: 89049127 | 21046 | 4: 92398977 | 21050 | 4: 92448883 |
| 21051 | 4: 92451008 | 21052 | 4: 92451165 | 21109 | 4: 93517701 | 21117 | 4: 93652019 | 21185 | 4: 95523178 |
| 21553 | 4: 100762424 | 21603 | 4: 101689426 | 21683 | 4: 103739455 | 21711 | 4: 104521615 | 21718 | 4: 104699947 |
| 21818 | 4: 106683233 | 21824 | 4: 106777370 | 21836 | 4: 106986917 | 21886 | 4: 107625627 | 21968 | 4: 109840544 |
| 22030 | 4: 111174187 | 22078 | 4: 111934020 | 22116 | 4: 112499124 | 22123 | 4: 112606237 | 22134 | 4: 112692746 |
| 22137 | 4: 112719377 | 22140 | 4: 112741810 | 22149 | 4: 112797073 | 22180 | 4: 113503432 | 22250 | 4: 114401519 |
| 22253 | 4: 114443077 | 22274 | 4: 114788184 | 22278 | 4: 114812550 | 22312 | 4: 115192347 | 22318 | 4: 115316340 |
| 22319 | 4: 115317642 | 22354 | 4: 116067653 | 22376 | 4: 116420553 | 22394 | 4: 116749463 | 22406 | 4: 116987390 |
| 22513 | 4: 119160825 | 22525 | 4: 119536985 | 22581 | 5: 882265 | 22670 | 5: 2514974 | 22693 | 5: 3086065 |
| 22707 | 5: 3684231 | 22731 | 5: 4371208 | 22740 | 5: 4517412 | 22761 | 5: 5025515 | 22776 | 5: 5256216 |
| 22899 | 5: 8017094 | 22935 | 5: 8963886 | 22937 | 5: 9058312 | 22939 | 5: 9081371 | 22941 | 5: 9110521 |
| 22974 | 5: 9917938 | 23184 | 5: 14901254 | 23203 | 5: 15246344 | 23335 | 5: 18879061 | 23340 | 5: 18957429 |
| 23448 | 5: 21803211 | 23511 | 5: 22947860 | 23513 | 5: 23010158 | 23563 | 5: 23986445 | 23619 | 5: 25424207 |
| 23628 | 5: 25663032 | 23683 | 5: 27040938 | 23685 | 5: 27062257 | 23767 | 5: 28682732 | 23795 | 5: 29481439 |
| 23894 | 5: 31935957 | 23904 | 5: 32259633 | 23908 | 5: 32334842 | 23922 | 5: 32649736 | 23951 | 5: 33311326 |
| 24015 | 5: 35004932 | 24046 | 5: 36022885 | 24051 | 5: 36113810 | 24053 | 5: 36193854 | 24092 | 5: 37455345 |
| 24105 | 5: 37749836 | 24243 | 5: 41293748 | 24244 | 5: 41337074 | 24247 | 5: 41451300 | 24254 | 5: 41682610 |
| 24265 | 5: 41958597 | 24268 | 5: 42010622 | 24275 | 5: 42382038 | 24295 | 5: 42883310 | 24301 | 5: 43024524 |
| 24312 | 5: 43371061 | 24352 | 5: 44118721 | 24387 | 5: 45222817 | 24450 | 5: 46743902 | 24683 | 5: 52698528 |
| 24690 | 5: 53009069 | 25020 | 5: 61964739 | 25024 | 5: 62044690 | 25041 | 5: 62542053 | 25049 | 5: 62915639 |
| 25350 | 5: 69917738 | 25398 | 5: 71197728 | 25467 | 5: 72417962 | 25469 | 5: 72465277 | 25527 | 5: 74079648 |
| 25639 | 5: 76766433 | 25642 | 5: 76818912 | 25644 | 5: 76890153 | 25645 | 5: 76972053 | 25757 | 5: 80114008 |
| 25760 | 5: 80235852 | 25784 | 5: 80982109 | 25787 | 5: 81116016 | 25814 | 5: 81935764 | 25875 | 5: 83029733 |
| 26074 | 5: 84527341 | 26206 | 5: 87607418 | 26217 | 5: 87838676 | 26304 | 5: 89147995 | 26364 | 5: 90614584 |
| 26395 | 5: 91363881 | 26508 | 5: 93490294 | 26558 | 5: 94637080 | 26603 | 5: 95645793 | 26605 | 5: 95684692 |
| 26607 | 5: 95699816 | 26610 | 5: 95747447 | 26611 | 5: 95748288 | 26612 | 5: 95749996 | 26763 | 5: 98601044 |
| 26855 | 5: 100383163 | 26876 | 5: 100815761 | 26879 | 5: 100854950 | 26917 | 5: 101625777 | 27085 | 5: 105474132 |
| 27094 | 5: 105732916 | 27095 | 5: 105733857 | 27096 | 5: 105740050 | 27097 | 5: 105756724 | 27098 | 5: 105760018 |
| 27099 | 5: 105760750 | 27101 | 5: 105770267 | 27102 | 5: 105771805 | 27103 | 5: 105773809 | 27104 | 5: 105776080 |
| 27105 | 5: 105776475 | 27106 | 5: 105778521 | 27107 | 5: 105778777 | 27108 | 5: 105779368 | 27109 | 5: 105780258 |
| 27110 | 5: 105800847 | 27112 | 5: 105804880 | 27167 | 5: 106540440 | 27175 | 5: 106650676 | 27227 | 5: 107423031 |
| 27378 | 5: 110192579 | 27381 | 5: 110289240 | 27391 | 5: 110386083 | 27419 | 5: 110751512 | 27501 | 5: 112519431 |
| 27502 | 5: 112553713 | 27533 | 5: 113205459 | 27568 | 5: 114190671 | 27822 | 5: 117930669 | 27862 | 5: 118720245 |
| 27891 | 5: 119036022 | 27910 | 5: 119441240 | 28029 | 6: 1993838 | 28117 | 6: 3589994 | 28156 | 6: 4424160 |
| 28249 | 6: 6458237 | 28250 | 6: 6482619 | 28394 | 6: 10017733 | 28447 | 6: 11463553 | 28470 | 6: 11875946 |
| 28484 | 6: 12191468 | 28503 | 6: 12548297 | 28504 | 6: 12550413 | 28512 | 6: 12753537 | 28513 | 6: 12808531 |
| 28515 | 6: 12873396 | 28517 | 6: 12900652 | 28580 | 6: 14515552 | 28582 | 6: 14546752 | 28584 | 6: 14593717 |
| 28588 | 6: 14737560 | 28660 | 6: 16069555 | 28665 | 6: 16173201 | 28670 | 6: 16334539 | 28671 | 6: 16387845 |
| 28672 | 6: 16407876 | 28700 | 6: 16918116 | 28708 | 6: 17036127 | 28721 | 6: 17373915 | 28727 | 6: 17498903 |
| 28786 | 6: 18598550 | 28800 | 6: 18917943 | 28802 | 6: 18955466 | 28828 | 6: 19455356 | 28838 | 6: 19628808 |
| 28847 | 6: 19848821 | 28851 | 6: 19923403 | 28858 | 6: 20135992 | 28864 | 6: 20273699 | 28873 | 6: 20399602 |
| 28881 | 6: 20600966 | 28884 | 6: 20625823 | 28891 | 6: 20779771 | 28892 | 6: 20821807 | 28906 | 6: 21288355 |
| 28909 | 6: 21362764 | 28911 | 6: 21407401 | 28920 | 6: 21614174 | 28924 | 6: 21682567 | 28929 | 6: 21732184 |
| 28930 | 6: 21736509 | 28937 | 6: 21857115 | 28943 | 6: 22080199 | 28948 | 6: 22169669 | 28958 | 6: 22265776 |
| 28982 | 6: 22743978 | 28986 | 6: 22838838 | 29009 | 6: 23503327 | 29017 | 6: 23665616 | 29019 | 6: 23697957 |
| 29025 | 6: 23885602 | 29026 | 6: 23902363 | 29027 | 6: 23902790 | 29037 | 6: 24156443 | 29038 | 6: 24188452 |
| 29062 | 6: 24764063 | 29073 | 6: 25006732 | 29074 | 6: 25007314 | 29075 | 6: 25007705 | 29085 | 6: 25188129 |
| 29088 | 6: 25333843 | 29095 | 6: 25528726 | 29104 | 6: 25843075 | 29112 | 6: 25978766 | 29117 | 6: 26022919 |
| 29125 | 6: 26158019 | 29142 | 6: 26679658 | 29146 | 6: 26760274 | 29148 | 6: 26792379 | 29156 | 6: 27042457 |
| 29160 | 6: 27154761 | 29165 | 6: 27332801 | 29171 | 6: 27406476 | 29192 | 6: 28048764 | 29202 | 6: 28285173 |
| 29219 | 6: 28873303 | 29221 | 6: 28936994 | 29222 | 6: 28940832 | 29224 | 6: 28978809 | 29229 | 6: 29062262 |
| 29242 | 6: 29357249 | 29246 | 6: 29406846 | 29252 | 6: 29478697 | 29261 | 6: 29591601 | 29264 | 6: 29642427 |
| 29266 | 6: 29700842 | 29272 | 6: 29850442 | 29275 | 6: 29928530 | 29278 | 6: 30059483 | 29281 | 6: 30087471 |
| 29282 | 6: 30092735 | 29283 | 6: 30096041 | 29284 | 6: 30096126 | 29285 | 6: 30100327 | 29286 | 6: 30101354 |
| 29288 | 6: 30105186 | 29289 | 6: 30107161 | 29290 | 6: 30107363 | 29291 | 6: 30107470 | 29292 | 6: 30107503 |
| 29293 | 6: 30108091 | 29294 | 6: 30108190 | 29295 | 6: 30108349 | 29296 | 6: 30108502 | 29297 | 6: 30109134 |
| 29298 | 6: 30109220 | 29299 | 6: 30109285 | 29300 | 6: 30109297 | 29301 | 6: 30109871 | 29302 | 6: 30109915 |
| 29303 | 6: 30110219 | 29304 | 6: 30110252 | 29305 | 6: 30110638 | 29306 | 6: 30111065 | 29307 | 6: 30111645 |
| 29308 | 6: 30112226 | 29309 | 6: 30112636 | 29310 | 6: 30112665 | 29311 | 6: 30112688 | 29312 | 6: 30112989 |
| 29313 | 6: 30113025 | 29314 | 6: 30113099 | 29315 | 6: 30113113 | 29316 | 6: 30113219 | 29317 | 6: 30113503 |
| 29318 | 6: 30113552 | 29319 | 6: 30113737 | 29320 | 6: 30114877 | 29321 | 6: 30114924 | 29322 | 6: 30115248 |
| 29323 | 6: 30115405 | 29324 | 6: 30115465 | 29325 | 6: 30117653 | 29326 | 6: 30117880 | 29327 | 6: 30117987 |
| 29328 | 6: 30118079 | 29329 | 6: 30118215 | 29330 | 6: 30119075 | 29331 | 6: 30119163 | 29332 | 6: 30119239 |
| 29333 | 6: 30119492 | 29334 | 6: 30120062 | 29335 | 6: 30120131 | 29336 | 6: 30120148 | 29337 | 6: 30120317 |
| 29338 | 6: 30120495 | 29339 | 6: 30120714 | 29340 | 6: 30120745 | 29341 | 6: 30121010 | 29345 | 6: 30186140 |
| 29347 | 6: 30231489 | 29348 | 6: 30231511 | 29349 | 6: 30231899 | 29350 | 6: 30231913 | 29351 | 6: 30232234 |
| 29356 | 6: 30429744 | 29366 | 6: 30575474 | 29370 | 6: 30706328 | 29373 | 6: 30820768 | 29381 | 6: 31067757 |
| 29386 | 6: 31258805 | 29390 | 6: 31380855 | 29392 | 6: 31425251 | 29394 | 6: 31479415 | 29397 | 6: 31502542 |
| 29398 | 6: 31523083 | 29409 | 6: 31802402 | 29417 | 6: 32011040 | 29446 | 6: 33106068 | 29447 | 6: 33200563 |
| 29448 | 6: 33249833 | 29450 | 6: 33303040 | 29454 | 6: 33451849 | 29456 | 6: 33555068 | 29466 | 6: 33983856 |
| 29474 | 6: 34086214 | 29476 | 6: 34139287 | 29477 | 6: 34174152 | 29478 | 6: 34179094 | 29479 | 6: 34185107 |
| 29480 | 6: 34189894 | 29481 | 6: 34197024 | 29483 | 6: 34206187 | 29484 | 6: 34207156 | 29485 | 6: 34210015 |
| 29487 | 6: 34227111 | 29488 | 6: 34229172 | 29489 | 6: 34236680 | 29491 | 6: 34238928 | 29492 | 6: 34239830 |
| 29493 | 6: 34248001 | 29494 | 6: 34262023 | 29495 | 6: 34291379 | 29496 | 6: 34292850 | 29497 | 6: 34293142 |
| 29498 | 6: 34297896 | 29499 | 6: 34298106 | 29500 | 6: 34300136 | 29501 | 6: 34305846 | 29502 | 6: 34313535 |
| 29503 | 6: 34314736 | 29504 | 6: 34316483 | 29506 | 6: 34317628 | 29507 | 6: 34317733 | 29508 | 6: 34319685 |
| 29509 | 6: 34319748 | 29510 | 6: 34319965 | 29511 | 6: 34320099 | 29512 | 6: 34320208 | 29513 | 6: 34320272 |
| 29514 | 6: 34320435 | 29515 | 6: 34320590 | 29516 | 6: 34320597 | 29517 | 6: 34321475 | 29518 | 6: 34321669 |
| 29519 | 6: 34321700 | 29520 | 6: 34322765 | 29521 | 6: 34323241 | 29522 | 6: 34323355 | 29523 | 6: 34323382 |
| 29524 | 6: 34323399 | 29525 | 6: 34323857 | 29526 | 6: 34323923 | 29527 | 6: 34324379 | 29528 | 6: 34324509 |
| 29529 | 6: 34324543 | 29530 | 6: 34324794 | 29531 | 6: 34324813 | 29532 | 6: 34324864 | 29533 | 6: 34325948 |
| 29534 | 6: 34326047 | 29535 | 6: 34326105 | 29536 | 6: 34326672 | 29537 | 6: 34326907 | 29538 | 6: 34326931 |
| 29539 | 6: 34327343 | 29540 | 6: 34328174 | 29541 | 6: 34328391 | 29542 | 6: 34328595 | 29543 | 6: 34328871 |
| 29544 | 6: 34329287 | 29545 | 6: 34329583 | 29546 | 6: 34329816 | 29547 | 6: 34329841 | 29548 | 6: 34329959 |
| 29549 | 6: 34330004 | 29550 | 6: 34330105 | 29551 | 6: 34330219 | 29552 | 6: 34330248 | 29553 | 6: 34330469 |
| 29554 | 6: 34330615 | 29555 | 6: 34331649 | 29556 | 6: 34331871 | 29557 | 6: 34334654 | 29558 | 6: 34334695 |
| 29559 | 6: 34334819 | 29560 | 6: 34334883 | 29561 | 6: 34334942 | 29562 | 6: 34334975 | 29563 | 6: 34335273 |
| 29564 | 6: 34335676 | 29565 | 6: 34336082 | 29566 | 6: 34336116 | 29567 | 6: 34336302 | 29568 | 6: 34336518 |
| 29569 | 6: 34336806 | 29570 | 6: 34337142 | 29571 | 6: 34337235 | 29572 | 6: 34337793 | 29573 | 6: 34337852 |
| 29574 | 6: 34337871 | 29575 | 6: 34337952 | 29576 | 6: 34338244 | 29577 | 6: 34338458 | 29578 | 6: 34338858 |
| 29579 | 6: 34338912 | 29580 | 6: 34341641 | 29581 | 6: 34341671 | 29582 | 6: 34342381 | 29583 | 6: 34342418 |
| 29584 | 6: 34342439 | 29585 | 6: 34342876 | 29586 | 6: 34343375 | 29587 | 6: 34344002 | 29588 | 6: 34344707 |
| 29589 | 6: 34344823 | 29590 | 6: 34344911 | 29591 | 6: 34345770 | 29592 | 6: 34345787 | 29593 | 6: 34347423 |
| 29594 | 6: 34347478 | 29595 | 6: 34347542 | 29596 | 6: 34347870 | 29597 | 6: 34347912 | 29598 | 6: 34348158 |
| 29599 | 6: 34348200 | 29600 | 6: 34350237 | 29601 | 6: 34350256 | 29602 | 6: 34350284 | 29603 | 6: 34352029 |
| 29604 | 6: 34352540 | 29605 | 6: 34353080 | 29606 | 6: 34353086 | 29607 | 6: 34353183 | 29608 | 6: 34353582 |
| 29609 | 6: 34353683 | 29610 | 6: 34355309 | 29611 | 6: 34356066 | 29612 | 6: 34356817 | 29613 | 6: 34357441 |
| 29614 | 6: 34357724 | 29615 | 6: 34358059 | 29616 | 6: 34358159 | 29617 | 6: 34359885 | 29618 | 6: 34359922 |
| 29619 | 6: 34360320 | 29620 | 6: 34360574 | 29621 | 6: 34360625 | 29622 | 6: 34361019 | 29623 | 6: 34361442 |
| 29624 | 6: 34362625 | 29625 | 6: 34363701 | 29626 | 6: 34366860 | 29627 | 6: 34367704 | 29628 | 6: 34368646 |
| 29629 | 6: 34368728 | 29630 | 6: 34368895 | 29631 | 6: 34369801 | 29632 | 6: 34370381 | 29633 | 6: 34373419 |
| 29634 | 6: 34375386 | 29635 | 6: 34377523 | 29636 | 6: 34378377 | 29637 | 6: 34378764 | 29638 | 6: 34379342 |
| 29639 | 6: 34379765 | 29640 | 6: 34380266 | 29641 | 6: 34380420 | 29642 | 6: 34390859 | 29643 | 6: 34398273 |
| 29644 | 6: 34400215 | 29645 | 6: 34401918 | 29646 | 6: 34405865 | 29647 | 6: 34409323 | 29648 | 6: 34412354 |
| 29649 | 6: 34412446 | 29650 | 6: 34417593 | 29651 | 6: 34423276 | 29652 | 6: 34425931 | 29653 | 6: 34429298 |
| 29654 | 6: 34433083 | 29655 | 6: 34436717 | 29656 | 6: 34443045 | 29657 | 6: 34445489 | 29658 | 6: 34454602 |
| 29661 | 6: 34485191 | 29663 | 6: 34508971 | 29664 | 6: 34511326 | 29665 | 6: 34512053 | 29666 | 6: 34512149 |
| 29667 | 6: 34512224 | 29668 | 6: 34512436 | 29669 | 6: 34512607 | 29670 | 6: 34514009 | 29671 | 6: 34520073 |
| 29672 | 6: 34520205 | 29673 | 6: 34520340 | 29674 | 6: 34521287 | 29675 | 6: 34521294 | 29676 | 6: 34522877 |
| 29677 | 6: 34523485 | 29678 | 6: 34524720 | 29679 | 6: 34526737 | 29680 | 6: 34527170 | 29681 | 6: 34531081 |
| 29682 | 6: 34531219 | 29683 | 6: 34531228 | 29684 | 6: 34532115 | 29685 | 6: 34532276 | 29686 | 6: 34533970 |
| 29687 | 6: 34534131 | 29688 | 6: 34534507 | 29689 | 6: 34534849 | 29690 | 6: 34534873 | 29691 | 6: 34535922 |
| 29692 | 6: 34535958 | 29693 | 6: 34535980 | 29694 | 6: 34537513 | 29695 | 6: 34538518 | 29696 | 6: 34538586 |
| 29697 | 6: 34538798 | 29698 | 6: 34539189 | 29699 | 6: 34544253 | 29700 | 6: 34544296 | 29701 | 6: 34544650 |
| 29702 | 6: 34544882 | 29703 | 6: 34544924 | 29704 | 6: 34545143 | 29705 | 6: 34545442 | 29706 | 6: 34545976 |
| 29707 | 6: 34546002 | 29708 | 6: 34546098 | 29709 | 6: 34546230 | 29710 | 6: 34546882 | 29711 | 6: 34551061 |
| 29712 | 6: 34551087 | 29713 | 6: 34551357 | 29714 | 6: 34551821 | 29715 | 6: 34552091 | 29716 | 6: 34552302 |
| 29717 | 6: 34559140 | 29719 | 6: 34568139 | 29720 | 6: 34570377 | 29721 | 6: 34571403 | 29723 | 6: 34585218 |
| 29724 | 6: 34587383 | 29726 | 6: 34590054 | 29727 | 6: 34590568 | 29728 | 6: 34590836 | 29729 | 6: 34590891 |
| 29730 | 6: 34594553 | 29731 | 6: 34595913 | 29732 | 6: 34605484 | 29734 | 6: 34606444 | 29735 | 6: 34606508 |
| 29736 | 6: 34607125 | 29737 | 6: 34607239 | 29738 | 6: 34608122 | 29739 | 6: 34608604 | 29740 | 6: 34609191 |
| 29741 | 6: 34609820 | 29742 | 6: 34609854 | 29743 | 6: 34610451 | 29744 | 6: 34610729 | 29745 | 6: 34610740 |
| 29746 | 6: 34610765 | 29748 | 6: 34610849 | 29749 | 6: 34611386 | 29750 | 6: 34611923 | 29751 | 6: 34611955 |
| 29752 | 6: 34612600 | 29753 | 6: 34613447 | 29754 | 6: 34614887 | 29755 | 6: 34615063 | 29756 | 6: 34617013 |
| 29757 | 6: 34617195 | 29758 | 6: 34617208 | 29759 | 6: 34617293 | 29760 | 6: 34617674 | 29761 | 6: 34617812 |
| 29762 | 6: 34617865 | 29763 | 6: 34617943 | 29764 | 6: 34618113 | 29765 | 6: 34618226 | 29766 | 6: 34618258 |
| 29767 | 6: 34618461 | 29768 | 6: 34618467 | 29769 | 6: 34618532 | 29770 | 6: 34618622 | 29771 | 6: 34618679 |
| 29772 | 6: 34618784 | 29773 | 6: 34618813 | 29774 | 6: 34618880 | 29775 | 6: 34620004 | 29776 | 6: 34620108 |
| 29777 | 6: 34620118 | 29778 | 6: 34620548 | 29779 | 6: 34621825 | 29780 | 6: 34622374 | 29781 | 6: 34623163 |
| 29782 | 6: 34623346 | 29783 | 6: 34623681 | 29784 | 6: 34623706 | 29785 | 6: 34623745 | 29786 | 6: 34624441 |
| 29787 | 6: 34629181 | 29788 | 6: 34629567 | 29789 | 6: 34629581 | 29790 | 6: 34629692 | 29791 | 6: 34629900 |
| 29792 | 6: 34629975 | 29793 | 6: 34629990 | 29794 | 6: 34630073 | 29795 | 6: 34630347 | 29796 | 6: 34630587 |
| 29797 | 6: 34630602 | 29798 | 6: 34630922 | 29799 | 6: 34631246 | 29800 | 6: 34631446 | 29801 | 6: 34631808 |
| 29802 | 6: 34631843 | 29803 | 6: 34632098 | 29804 | 6: 34632375 | 29805 | 6: 34632405 | 29806 | 6: 34632714 |
| 29807 | 6: 34632833 | 29808 | 6: 34633050 | 29809 | 6: 34633159 | 29810 | 6: 34633187 | 29811 | 6: 34633229 |
| 29812 | 6: 34633562 | 29813 | 6: 34633782 | 29814 | 6: 34633841 | 29815 | 6: 34633967 | 29816 | 6: 34633990 |
| 29817 | 6: 34634055 | 29818 | 6: 34634169 | 29819 | 6: 34634190 | 29820 | 6: 34634250 | 29821 | 6: 34634312 |
| 29822 | 6: 34634801 | 29823 | 6: 34634990 | 29824 | 6: 34635003 | 29825 | 6: 34635530 | 29826 | 6: 34635703 |
| 29827 | 6: 34636131 | 29828 | 6: 34636138 | 29829 | 6: 34636209 | 29830 | 6: 34637085 | 29831 | 6: 34640553 |
| 29832 | 6: 34641695 | 29833 | 6: 34647407 | 29834 | 6: 34657657 | 29835 | 6: 34661296 | 29836 | 6: 34661323 |
| 29837 | 6: 34662849 | 29838 | 6: 34665463 | 29839 | 6: 34668000 | 29840 | 6: 34670574 | 29841 | 6: 34671333 |
| 29842 | 6: 34673238 | 29844 | 6: 34686618 | 29845 | 6: 34687343 | 29846 | 6: 34689725 | 29847 | 6: 34697260 |
| 29848 | 6: 34699079 | 29849 | 6: 34703681 | 29850 | 6: 34704601 | 29851 | 6: 34704806 | 29852 | 6: 34705455 |
| 29853 | 6: 34714302 | 29854 | 6: 34714956 | 29855 | 6: 34724151 | 29856 | 6: 34726853 | 29857 | 6: 34727995 |
| 29858 | 6: 34728449 | 29859 | 6: 34730148 | 29860 | 6: 34730862 | 29861 | 6: 34734530 | 29863 | 6: 34738528 |
| 29864 | 6: 34746517 | 29865 | 6: 34751152 | 29866 | 6: 34751196 | 29867 | 6: 34751889 | 29868 | 6: 34753115 |
| 29869 | 6: 34762474 | 29870 | 6: 34762792 | 29871 | 6: 34762888 | 29872 | 6: 34766154 | 29873 | 6: 34766590 |
| 29874 | 6: 34766756 | 29875 | 6: 34768280 | 29876 | 6: 34768301 | 29877 | 6: 34768997 | 29878 | 6: 34769036 |
| 29879 | 6: 34769720 | 29880 | 6: 34770109 | 29881 | 6: 34770808 | 29882 | 6: 34777728 | 29883 | 6: 34780553 |
| 29884 | 6: 34781550 | 29885 | 6: 34781934 | 29886 | 6: 34782739 | 29887 | 6: 34782759 | 29888 | 6: 34783042 |
| 29889 | 6: 34783961 | 29890 | 6: 34784243 | 29891 | 6: 34784299 | 29892 | 6: 34784335 | 29893 | 6: 34784977 |
| 29894 | 6: 34785811 | 29895 | 6: 34785864 | 29896 | 6: 34785916 | 29897 | 6: 34786001 | 29898 | 6: 34786006 |
| 29899 | 6: 34786198 | 29900 | 6: 34786203 | 29901 | 6: 34786474 | 29902 | 6: 34786586 | 29903 | 6: 34787240 |
| 29904 | 6: 34789109 | 29905 | 6: 34792867 | 29907 | 6: 34845861 | 29909 | 6: 34929550 | 29910 | 6: 34932019 |
| 29911 | 6: 34933373 | 29912 | 6: 34943666 | 29913 | 6: 34961634 | 29915 | 6: 34972640 | 29916 | 6: 34976990 |
| 29917 | 6: 34985119 | 29918 | 6: 34990679 | 29919 | 6: 34992674 | 29920 | 6: 34994097 | 29921 | 6: 34995220 |
| 29922 | 6: 35021191 | 29923 | 6: 35047210 | 29924 | 6: 35140608 | 29927 | 6: 35157410 | 29929 | 6: 35160353 |
| 29930 | 6: 35161520 | 29932 | 6: 35162419 | 29934 | 6: 35163337 | 29935 | 6: 35165014 | 29936 | 6: 35166488 |
| 29937 | 6: 35166725 | 29942 | 6: 35245781 | 29945 | 6: 35274129 | 29948 | 6: 35381936 | 29953 | 6: 35504728 |
| 29957 | 6: 35561016 | 29958 | 6: 35561546 | 29959 | 6: 35561595 | 29960 | 6: 35581628 | 29961 | 6: 35582162 |
| 29962 | 6: 35582223 | 29963 | 6: 35587094 | 29964 | 6: 35594486 | 29965 | 6: 35609063 | 29966 | 6: 35616265 |
| 29967 | 6: 35616280 | 29969 | 6: 35618707 | 29974 | 6: 35666246 | 29975 | 6: 35678704 | 29976 | 6: 35678754 |
| 29977 | 6: 35684254 | 29978 | 6: 35703001 | 29979 | 6: 35713846 | 29980 | 6: 35718341 | 29981 | 6: 35726574 |
| 29982 | 6: 35732398 | 29984 | 6: 35792391 | 29985 | 6: 35825879 | 29987 | 6: 35886281 | 29989 | 6: 35897314 |
| 29994 | 6: 35908980 | 30001 | 6: 35937425 | 30002 | 6: 35937819 | 30003 | 6: 35937874 | 30004 | 6: 35941557 |
| 30006 | 6: 35948457 | 30007 | 6: 35963118 | 30008 | 6: 35968631 | 30009 | 6: 35972831 | 30012 | 6: 35992028 |
| 30014 | 6: 36011027 | 30015 | 6: 36011772 | 30018 | 6: 36032905 | 30019 | 6: 36034678 | 30020 | 6: 36036595 |
| 30029 | 6: 36125455 | 30030 | 6: 36143409 | 30031 | 6: 36145256 | 30032 | 6: 36147418 | 30033 | 6: 36148375 |
| 30034 | 6: 36149041 | 30035 | 6: 36150876 | 30036 | 6: 36151325 | 30037 | 6: 36153013 | 30038 | 6: 36156606 |
| 30039 | 6: 36156894 | 30040 | 6: 36157622 | 30041 | 6: 36157988 | 30042 | 6: 36158361 | 30043 | 6: 36158500 |
| 30044 | 6: 36159608 | 30045 | 6: 36161275 | 30046 | 6: 36161377 | 30047 | 6: 36161627 | 30048 | 6: 36161635 |
| 30049 | 6: 36162952 | 30050 | 6: 36163342 | 30051 | 6: 36163430 | 30052 | 6: 36164022 | 30053 | 6: 36164566 |
| 30054 | 6: 36165003 | 30055 | 6: 36165139 | 30056 | 6: 36165152 | 30057 | 6: 36165220 | 30058 | 6: 36165307 |
| 30059 | 6: 36166110 | 30060 | 6: 36166180 | 30061 | 6: 36166207 | 30062 | 6: 36166505 | 30063 | 6: 36166793 |
| 30064 | 6: 36166797 | 30065 | 6: 36167327 | 30066 | 6: 36167830 | 30067 | 6: 36168126 | 30068 | 6: 36168722 |
| 30069 | 6: 36169714 | 30070 | 6: 36169794 | 30071 | 6: 36170135 | 30072 | 6: 36170201 | 30073 | 6: 36171686 |
| 30074 | 6: 36172372 | 30075 | 6: 36177201 | 30076 | 6: 36180645 | 30077 | 6: 36181021 | 30078 | 6: 36182794 |
| 30079 | 6: 36183913 | 30080 | 6: 36186583 | 30081 | 6: 36191256 | 30082 | 6: 36198702 | 30083 | 6: 36204036 |
| 30084 | 6: 36211597 | 30085 | 6: 36212186 | 30086 | 6: 36219844 | 30087 | 6: 36225618 | 30088 | 6: 36236546 |
| 30089 | 6: 36238601 | 30090 | 6: 36238793 | 30091 | 6: 36239080 | 30092 | 6: 36239773 | 30093 | 6: 36240264 |
| 30094 | 6: 36241246 | 30095 | 6: 36246325 | 30096 | 6: 36248262 | 30097 | 6: 36249143 | 30098 | 6: 36250084 |
| 30099 | 6: 36254264 | 30100 | 6: 36255334 | 30101 | 6: 36256227 | 30102 | 6: 36256264 | 30103 | 6: 36256316 |
| 30104 | 6: 36261278 | 30105 | 6: 36262429 | 30106 | 6: 36263719 | 30107 | 6: 36264671 | 30108 | 6: 36264792 |
| 30109 | 6: 36265379 | 30110 | 6: 36272248 | 30111 | 6: 36273670 | 30112 | 6: 36277826 | 30113 | 6: 36280253 |
| 30114 | 6: 36283110 | 30115 | 6: 36283198 | 30116 | 6: 36286200 | 30117 | 6: 36292811 | 30118 | 6: 36298957 |
| 30119 | 6: 36299463 | 30120 | 6: 36299867 | 30121 | 6: 36300007 | 30122 | 6: 36324361 | 30123 | 6: 36333247 |
| 30124 | 6: 36336622 | 30125 | 6: 36336765 | 30126 | 6: 36337366 | 30127 | 6: 36340265 | 30128 | 6: 36363919 |
| 30129 | 6: 36365275 | 30131 | 6: 36412092 | 30141 | 6: 36436269 | 30142 | 6: 36440409 | 30143 | 6: 36441375 |
| 30144 | 6: 36443819 | 30146 | 6: 36445226 | 30149 | 6: 36454421 | 30151 | 6: 36457471 | 30152 | 6: 36457509 |
| 30153 | 6: 36458519 | 30154 | 6: 36460784 | 30155 | 6: 36461234 | 30156 | 6: 36461574 | 30157 | 6: 36463931 |
| 30158 | 6: 36466353 | 30159 | 6: 36466871 | 30161 | 6: 36469381 | 30162 | 6: 36469975 | 30163 | 6: 36474704 |
| 30166 | 6: 36478628 | 30167 | 6: 36480305 | 30170 | 6: 36482113 | 30172 | 6: 36487754 | 30173 | 6: 36489160 |
| 30176 | 6: 36491203 | 30177 | 6: 36491990 | 30178 | 6: 36499104 | 30179 | 6: 36499659 | 30180 | 6: 36500389 |
| 30181 | 6: 36501474 | 30182 | 6: 36503509 | 30183 | 6: 36506439 | 30184 | 6: 36506901 | 30185 | 6: 36509073 |
| 30186 | 6: 36514666 | 30187 | 6: 36514687 | 30188 | 6: 36516071 | 30189 | 6: 36518117 | 30190 | 6: 36524445 |
| 30191 | 6: 36524489 | 30192 | 6: 36529972 | 30193 | 6: 36530565 | 30194 | 6: 36531251 | 30195 | 6: 36531630 |
| 30197 | 6: 36532751 | 30198 | 6: 36533529 | 30199 | 6: 36533756 | 30200 | 6: 36533947 | 30201 | 6: 36534891 |
| 30202 | 6: 36535012 | 30203 | 6: 36535227 | 30204 | 6: 36535356 | 30205 | 6: 36535863 | 30206 | 6: 36535904 |
| 30207 | 6: 36536565 | 30208 | 6: 36537055 | 30209 | 6: 36537210 | 30210 | 6: 36537223 | 30211 | 6: 36537284 |
| 30212 | 6: 36537515 | 30213 | 6: 36537657 | 30214 | 6: 36537708 | 30215 | 6: 36538044 | 30216 | 6: 36538107 |
| 30217 | 6: 36538113 | 30218 | 6: 36538805 | 30219 | 6: 36538827 | 30220 | 6: 36539051 | 30221 | 6: 36539508 |
| 30222 | 6: 36539630 | 30223 | 6: 36540193 | 30224 | 6: 36540734 | 30225 | 6: 36540815 | 30226 | 6: 36543846 |
| 30227 | 6: 36544549 | 30228 | 6: 36544681 | 30229 | 6: 36544687 | 30230 | 6: 36544786 | 30231 | 6: 36544914 |
| 30232 | 6: 36545202 | 30233 | 6: 36545304 | 30235 | 6: 36547031 | 30236 | 6: 36547078 | 30237 | 6: 36547888 |
| 30238 | 6: 36547972 | 30239 | 6: 36548012 | 30240 | 6: 36548205 | 30241 | 6: 36548953 | 30242 | 6: 36549261 |
| 30243 | 6: 36549683 | 30244 | 6: 36550069 | 30245 | 6: 36550199 | 30246 | 6: 36551621 | 30247 | 6: 36552037 |
| 30248 | 6: 36552742 | 30249 | 6: 36552915 | 30252 | 6: 36556271 | 30253 | 6: 36556442 | 30254 | 6: 36557671 |
| 30256 | 6: 36561952 | 30258 | 6: 36567616 | 30262 | 6: 36569777 | 30263 | 6: 36571430 | 30265 | 6: 36594692 |
| 30266 | 6: 36598347 | 30267 | 6: 36599192 | 30268 | 6: 36602971 | 30269 | 6: 36603958 | 30270 | 6: 36608521 |
| 30271 | 6: 36608642 | 30272 | 6: 36611873 | 30273 | 6: 36612067 | 30274 | 6: 36614641 | 30275 | 6: 36617150 |
| 30276 | 6: 36618712 | 30277 | 6: 36619708 | 30278 | 6: 36620654 | 30279 | 6: 36621895 | 30280 | 6: 36622608 |
| 30281 | 6: 36624334 | 30282 | 6: 36625203 | 30283 | 6: 36625485 | 30285 | 6: 36626119 | 30286 | 6: 36626509 |
| 30287 | 6: 36629387 | 30288 | 6: 36631261 | 30289 | 6: 36632276 | 30290 | 6: 36632779 | 30291 | 6: 36633630 |
| 30292 | 6: 36633929 | 30293 | 6: 36634989 | 30294 | 6: 36635404 | 30295 | 6: 36635605 | 30296 | 6: 36635939 |
| 30297 | 6: 36638521 | 30298 | 6: 36638963 | 30299 | 6: 36639106 | 30300 | 6: 36642083 | 30301 | 6: 36642499 |
| 30302 | 6: 36643100 | 30303 | 6: 36643378 | 30304 | 6: 36644151 | 30305 | 6: 36645330 | 30306 | 6: 36646194 |
| 30307 | 6: 36646596 | 30308 | 6: 36648068 | 30309 | 6: 36648160 | 30310 | 6: 36653563 | 30311 | 6: 36657483 |
| 30312 | 6: 36664663 | 30313 | 6: 36673216 | 30314 | 6: 36679692 | 30316 | 6: 36681772 | 30318 | 6: 36695293 |
| 30319 | 6: 36700131 | 30320 | 6: 36700135 | 30321 | 6: 36706370 | 30322 | 6: 36720331 | 30324 | 6: 36743854 |
| 30325 | 6: 36773960 | 30326 | 6: 36787210 | 30331 | 6: 36812951 | 30332 | 6: 36813102 | 30333 | 6: 36813124 |
| 30334 | 6: 36813128 | 30335 | 6: 36813593 | 30336 | 6: 36813633 | 30337 | 6: 36814581 | 30338 | 6: 36814786 |
| 30339 | 6: 36815144 | 30340 | 6: 36815200 | 30341 | 6: 36815490 | 30342 | 6: 36815725 | 30343 | 6: 36815817 |
| 30344 | 6: 36816067 | 30345 | 6: 36816314 | 30346 | 6: 36816419 | 30347 | 6: 36817159 | 30348 | 6: 36817266 |
| 30349 | 6: 36817581 | 30350 | 6: 36817596 | 30351 | 6: 36817683 | 30352 | 6: 36817713 | 30353 | 6: 36817969 |
| 30354 | 6: 36818237 | 30355 | 6: 36818704 | 30356 | 6: 36818808 | 30357 | 6: 36819594 | 30358 | 6: 36819695 |
| 30359 | 6: 36819751 | 30360 | 6: 36820074 | 30361 | 6: 36820138 | 30362 | 6: 36820361 | 30363 | 6: 36820770 |
| 30364 | 6: 36821432 | 30365 | 6: 36821474 | 30366 | 6: 36822273 | 30367 | 6: 36822526 | 30368 | 6: 36822568 |
| 30369 | 6: 36822744 | 30370 | 6: 36823102 | 30371 | 6: 36823606 | 30372 | 6: 36825509 | 30373 | 6: 36830103 |
| 30374 | 6: 36830934 | 30375 | 6: 36831012 | 30376 | 6: 36831131 | 30377 | 6: 36831274 | 30378 | 6: 36831813 |
| 30379 | 6: 36831871 | 30380 | 6: 36834692 | 30381 | 6: 36835108 | 30391 | 6: 36949956 | 30392 | 6: 36952579 |
| 30393 | 6: 36954127 | 30395 | 6: 36959305 | 30396 | 6: 36960146 | 30397 | 6: 36970451 | 30398 | 6: 36978207 |
| 30399 | 6: 36978216 | 30400 | 6: 36978361 | 30401 | 6: 36978371 | 30402 | 6: 36978422 | 30403 | 6: 36978431 |
| 30404 | 6: 36978698 | 30405 | 6: 36978703 | 30406 | 6: 36978958 | 30407 | 6: 36979025 | 30408 | 6: 36979081 |
| 30409 | 6: 36979093 | 30410 | 6: 36979116 | 30411 | 6: 36979134 | 30412 | 6: 36979146 | 30413 | 6: 36981664 |
| 30414 | 6: 36981815 | 30415 | 6: 36985783 | 30416 | 6: 36985804 | 30417 | 6: 36995904 | 30419 | 6: 37010986 |
| 30420 | 6: 37011036 | 30421 | 6: 37016748 | 30422 | 6: 37017628 | 30423 | 6: 37017833 | 30424 | 6: 37018373 |
| 30425 | 6: 37018495 | 30426 | 6: 37018672 | 30427 | 6: 37018745 | 30428 | 6: 37018878 | 30429 | 6: 37019035 |
| 30430 | 6: 37019263 | 30431 | 6: 37019274 | 30432 | 6: 37019373 | 30433 | 6: 37019415 | 30434 | 6: 37019464 |
| 30435 | 6: 37019512 | 30436 | 6: 37019519 | 30437 | 6: 37019606 | 30438 | 6: 37020100 | 30439 | 6: 37020244 |
| 30440 | 6: 37020255 | 30441 | 6: 37020456 | 30442 | 6: 37020574 | 30443 | 6: 37020591 | 30444 | 6: 37020608 |
| 30445 | 6: 37021123 | 30446 | 6: 37021154 | 30447 | 6: 37021520 | 30448 | 6: 37021604 | 30449 | 6: 37021714 |
| 30450 | 6: 37021968 | 30451 | 6: 37022137 | 30452 | 6: 37022181 | 30453 | 6: 37022238 | 30454 | 6: 37023645 |
| 30460 | 6: 37030480 | 30461 | 6: 37032906 | 30463 | 6: 37034096 | 30464 | 6: 37034286 | 30465 | 6: 37034823 |
| 30466 | 6: 37034912 | 30467 | 6: 37035186 | 30469 | 6: 37038017 | 30470 | 6: 37044068 | 30473 | 6: 37061527 |
| 30474 | 6: 37074563 | 30475 | 6: 37075910 | 30476 | 6: 37078470 | 30477 | 6: 37078665 | 30478 | 6: 37078781 |
| 30479 | 6: 37079090 | 30480 | 6: 37079147 | 30481 | 6: 37079324 | 30482 | 6: 37079422 | 30483 | 6: 37079559 |
| 30484 | 6: 37079653 | 30485 | 6: 37079666 | 30486 | 6: 37080062 | 30487 | 6: 37080097 | 30488 | 6: 37080210 |
| 30489 | 6: 37080486 | 30490 | 6: 37080919 | 30491 | 6: 37080936 | 30492 | 6: 37081034 | 30493 | 6: 37081117 |
| 30494 | 6: 37081212 | 30495 | 6: 37081319 | 30496 | 6: 37081745 | 30497 | 6: 37081947 | 30498 | 6: 37082116 |
| 30499 | 6: 37082537 | 30500 | 6: 37082542 | 30501 | 6: 37082592 | 30502 | 6: 37082641 | 30503 | 6: 37082764 |
| 30504 | 6: 37082830 | 30505 | 6: 37083023 | 30506 | 6: 37083086 | 30507 | 6: 37083196 | 30508 | 6: 37083881 |
| 30509 | 6: 37084518 | 30510 | 6: 37084543 | 30511 | 6: 37084979 | 30512 | 6: 37084987 | 30513 | 6: 37084991 |
| 30514 | 6: 37085423 | 30515 | 6: 37085516 | 30516 | 6: 37087274 | 30517 | 6: 37087642 | 30518 | 6: 37087987 |
| 30519 | 6: 37087999 | 30520 | 6: 37088359 | 30521 | 6: 37088506 | 30522 | 6: 37088510 | 30523 | 6: 37088831 |
| 30524 | 6: 37088835 | 30525 | 6: 37088955 | 30526 | 6: 37089045 | 30527 | 6: 37089108 | 30528 | 6: 37089125 |
| 30529 | 6: 37089202 | 30530 | 6: 37089209 | 30531 | 6: 37089234 | 30532 | 6: 37089651 | 30533 | 6: 37089673 |
| 30534 | 6: 37089763 | 30535 | 6: 37089819 | 30536 | 6: 37089854 | 30537 | 6: 37089926 | 30538 | 6: 37090001 |
| 30539 | 6: 37090127 | 30540 | 6: 37090147 | 30541 | 6: 37090190 | 30542 | 6: 37090229 | 30543 | 6: 37090257 |
| 30544 | 6: 37090343 | 30545 | 6: 37090838 | 30546 | 6: 37090865 | 30547 | 6: 37091359 | 30548 | 6: 37091365 |
| 30549 | 6: 37091616 | 30550 | 6: 37091767 | 30551 | 6: 37092094 | 30552 | 6: 37092116 | 30553 | 6: 37092227 |
| 30554 | 6: 37092573 | 30555 | 6: 37092869 | 30556 | 6: 37092904 | 30557 | 6: 37092936 | 30558 | 6: 37093073 |
| 30559 | 6: 37093934 | 30560 | 6: 37093949 | 30561 | 6: 37094212 | 30562 | 6: 37094603 | 30563 | 6: 37094693 |
| 30564 | 6: 37094789 | 30565 | 6: 37094827 | 30566 | 6: 37095299 | 30567 | 6: 37095517 | 30568 | 6: 37096027 |
| 30569 | 6: 37096074 | 30570 | 6: 37096486 | 30571 | 6: 37096495 | 30572 | 6: 37096649 | 30573 | 6: 37096879 |
| 30574 | 6: 37096959 | 30575 | 6: 37097509 | 30576 | 6: 37097522 | 30577 | 6: 37097635 | 30578 | 6: 37097772 |
| 30579 | 6: 37097995 | 30580 | 6: 37098052 | 30581 | 6: 37098220 | 30582 | 6: 37098309 | 30583 | 6: 37098640 |
| 30584 | 6: 37098716 | 30585 | 6: 37098782 | 30586 | 6: 37098831 | 30587 | 6: 37098872 | 30588 | 6: 37099284 |
| 30589 | 6: 37099535 | 30590 | 6: 37099560 | 30591 | 6: 37099942 | 30592 | 6: 37099978 | 30593 | 6: 37099983 |
| 30594 | 6: 37100199 | 30595 | 6: 37100203 | 30596 | 6: 37100223 | 30597 | 6: 37100242 | 30598 | 6: 37100749 |
| 30599 | 6: 37100852 | 30600 | 6: 37100906 | 30601 | 6: 37100924 | 30602 | 6: 37101114 | 30603 | 6: 37101213 |
| 30604 | 6: 37101414 | 30605 | 6: 37101664 | 30606 | 6: 37101691 | 30607 | 6: 37104751 | 30608 | 6: 37104774 |
| 30609 | 6: 37104851 | 30610 | 6: 37104926 | 30611 | 6: 37104993 | 30612 | 6: 37105044 | 30613 | 6: 37105252 |
| 30614 | 6: 37105257 | 30615 | 6: 37106212 | 30616 | 6: 37107589 | 30617 | 6: 37107774 | 30618 | 6: 37108028 |
| 30619 | 6: 37108237 | 30620 | 6: 37112827 | 30621 | 6: 37112864 | 30622 | 6: 37116160 | 30623 | 6: 37116221 |
| 30624 | 6: 37116481 | 30625 | 6: 37116730 | 30626 | 6: 37117771 | 30627 | 6: 37117988 | 30628 | 6: 37120342 |
| 30630 | 6: 37123023 | 30631 | 6: 37128820 | 30634 | 6: 37136040 | 30635 | 6: 37136706 | 30636 | 6: 37136794 |
| 30637 | 6: 37138117 | 30638 | 6: 37138139 | 30639 | 6: 37138262 | 30640 | 6: 37138535 | 30641 | 6: 37139425 |
| 30642 | 6: 37140287 | 30643 | 6: 37142174 | 30644 | 6: 37142953 | 30645 | 6: 37144122 | 30646 | 6: 37147544 |
| 30647 | 6: 37147627 | 30648 | 6: 37149236 | 30649 | 6: 37149945 | 30650 | 6: 37150579 | 30651 | 6: 37151586 |
| 30652 | 6: 37151618 | 30654 | 6: 37152332 | 30656 | 6: 37153197 | 30657 | 6: 37153310 | 30658 | 6: 37153398 |
| 30659 | 6: 37153558 | 30660 | 6: 37153831 | 30661 | 6: 37155597 | 30662 | 6: 37156064 | 30663 | 6: 37156337 |
| 30664 | 6: 37156677 | 30665 | 6: 37156889 | 30666 | 6: 37157333 | 30667 | 6: 37157660 | 30668 | 6: 37158136 |
| 30669 | 6: 37158289 | 30670 | 6: 37158384 | 30671 | 6: 37159298 | 30672 | 6: 37159341 | 30673 | 6: 37159410 |
| 30674 | 6: 37159427 | 30675 | 6: 37159591 | 30676 | 6: 37159718 | 30677 | 6: 37159778 | 30678 | 6: 37159892 |
| 30679 | 6: 37160005 | 30680 | 6: 37160134 | 30681 | 6: 37160139 | 30682 | 6: 37160559 | 30683 | 6: 37161039 |
| 30684 | 6: 37161146 | 30685 | 6: 37161408 | 30686 | 6: 37161619 | 30687 | 6: 37161712 | 30688 | 6: 37162703 |
| 30689 | 6: 37162813 | 30690 | 6: 37162822 | 30691 | 6: 37163101 | 30692 | 6: 37163223 | 30693 | 6: 37163776 |
| 30694 | 6: 37163934 | 30695 | 6: 37164094 | 30696 | 6: 37164218 | 30697 | 6: 37164439 | 30698 | 6: 37164529 |
| 30699 | 6: 37164825 | 30700 | 6: 37165013 | 30701 | 6: 37165306 | 30702 | 6: 37165536 | 30703 | 6: 37165545 |
| 30704 | 6: 37165588 | 30705 | 6: 37165831 | 30706 | 6: 37165836 | 30707 | 6: 37166157 | 30708 | 6: 37166449 |
| 30709 | 6: 37166482 | 30710 | 6: 37167166 | 30711 | 6: 37167349 | 30712 | 6: 37167472 | 30713 | 6: 37167487 |
| 30714 | 6: 37167778 | 30715 | 6: 37167868 | 30716 | 6: 37167910 | 30717 | 6: 37167946 | 30718 | 6: 37168208 |
| 30719 | 6: 37168379 | 30720 | 6: 37168414 | 30721 | 6: 37168500 | 30722 | 6: 37168914 | 30723 | 6: 37168959 |
| 30724 | 6: 37169024 | 30725 | 6: 37169066 | 30726 | 6: 37169247 | 30727 | 6: 37169728 | 30728 | 6: 37169746 |
| 30729 | 6: 37169824 | 30730 | 6: 37171398 | 30731 | 6: 37171831 | 30732 | 6: 37172063 | 30733 | 6: 37172194 |
| 30734 | 6: 37172300 | 30736 | 6: 37173048 | 30737 | 6: 37173169 | 30738 | 6: 37173382 | 30739 | 6: 37174433 |
| 30740 | 6: 37174739 | 30741 | 6: 37174812 | 30742 | 6: 37174838 | 30743 | 6: 37174847 | 30744 | 6: 37174944 |
| 30745 | 6: 37175216 | 30746 | 6: 37175405 | 30747 | 6: 37175422 | 30749 | 6: 37175709 | 30751 | 6: 37175915 |
| 30752 | 6: 37175930 | 30753 | 6: 37175957 | 30754 | 6: 37176060 | 30755 | 6: 37176229 | 30756 | 6: 37176528 |
| 30758 | 6: 37176686 | 30759 | 6: 37177441 | 30760 | 6: 37177466 | 30761 | 6: 37177638 | 30762 | 6: 37177788 |
| 30763 | 6: 37177792 | 30764 | 6: 37177809 | 30765 | 6: 37178122 | 30766 | 6: 37178336 | 30767 | 6: 37178722 |
| 30768 | 6: 37179535 | 30769 | 6: 37180932 | 30770 | 6: 37180966 | 30771 | 6: 37181059 | 30772 | 6: 37181087 |
| 30773 | 6: 37181206 | 30774 | 6: 37181374 | 30775 | 6: 37181533 | 30776 | 6: 37181894 | 30777 | 6: 37182068 |
| 30778 | 6: 37182373 | 30779 | 6: 37182427 | 30780 | 6: 37182621 | 30782 | 6: 37182810 | 30783 | 6: 37182983 |
| 30784 | 6: 37183090 | 30785 | 6: 37183609 | 30786 | 6: 37183649 | 30789 | 6: 37185316 | 30790 | 6: 37188198 |
| 30793 | 6: 37191616 | 30795 | 6: 37193241 | 30796 | 6: 37195555 | 30800 | 6: 37211057 | 30801 | 6: 37214068 |
| 30802 | 6: 37214389 | 30803 | 6: 37214736 | 30804 | 6: 37216084 | 30805 | 6: 37216293 | 30806 | 6: 37217600 |
| 30807 | 6: 37218354 | 30808 | 6: 37218423 | 30809 | 6: 37219372 | 30811 | 6: 37221337 | 30812 | 6: 37221507 |
| 30813 | 6: 37221776 | 30814 | 6: 37222424 | 30815 | 6: 37225729 | 30816 | 6: 37226016 | 30817 | 6: 37226725 |
| 30819 | 6: 37228661 | 30821 | 6: 37231769 | 30822 | 6: 37232090 | 30823 | 6: 37232142 | 30824 | 6: 37232577 |
| 30825 | 6: 37233429 | 30826 | 6: 37233659 | 30827 | 6: 37234042 | 30828 | 6: 37234954 | 30831 | 6: 37236226 |
| 30832 | 6: 37237969 | 30833 | 6: 37238128 | 30834 | 6: 37238402 | 30835 | 6: 37238486 | 30836 | 6: 37238502 |
| 30837 | 6: 37238713 | 30838 | 6: 37238946 | 30839 | 6: 37241792 | 30840 | 6: 37241812 | 30841 | 6: 37243159 |
| 30842 | 6: 37243373 | 30843 | 6: 37243677 | 30845 | 6: 37244804 | 30846 | 6: 37245026 | 30847 | 6: 37245138 |
| 30849 | 6: 37247874 | 30850 | 6: 37248754 | 30853 | 6: 37251182 | 30854 | 6: 37252978 | 30855 | 6: 37255945 |
| 30860 | 6: 37263319 | 30861 | 6: 37264863 | 30862 | 6: 37265148 | 30863 | 6: 37266735 | 30866 | 6: 37270930 |
| 30868 | 6: 37273774 | 30869 | 6: 37278799 | 30870 | 6: 37281308 | 30871 | 6: 37281335 | 30872 | 6: 37282356 |
| 30873 | 6: 37284874 | 30874 | 6: 37285174 | 30875 | 6: 37285822 | 30876 | 6: 37285843 | 30877 | 6: 37286325 |
| 30878 | 6: 37286380 | 30880 | 6: 37286722 | 30889 | 6: 37289572 | 30904 | 6: 37293699 | 30906 | 6: 37294860 |
| 30907 | 6: 37295049 | 30908 | 6: 37295154 | 30909 | 6: 37295245 | 30910 | 6: 37295403 | 30911 | 6: 37295803 |
| 30912 | 6: 37296114 | 30913 | 6: 37297426 | 30914 | 6: 37297697 | 30915 | 6: 37298251 | 30916 | 6: 37298617 |
| 30917 | 6: 37298725 | 30918 | 6: 37298786 | 30919 | 6: 37299349 | 30920 | 6: 37299587 | 30921 | 6: 37299855 |
| 30922 | 6: 37299968 | 30923 | 6: 37300447 | 30924 | 6: 37300499 | 30925 | 6: 37300590 | 30926 | 6: 37300767 |
| 30927 | 6: 37300810 | 30928 | 6: 37300872 | 30929 | 6: 37301720 | 30930 | 6: 37301762 | 30931 | 6: 37301893 |
| 30932 | 6: 37301959 | 30933 | 6: 37302011 | 30934 | 6: 37302172 | 30935 | 6: 37302478 | 30936 | 6: 37302499 |
| 30937 | 6: 37302730 | 30938 | 6: 37303255 | 30939 | 6: 37303264 | 30940 | 6: 37303500 | 30941 | 6: 37303542 |
| 30942 | 6: 37303727 | 30943 | 6: 37304109 | 30944 | 6: 37304324 | 30945 | 6: 37304493 | 30947 | 6: 37304564 |
| 30948 | 6: 37304601 | 30949 | 6: 37304640 | 30950 | 6: 37304825 | 30951 | 6: 37305062 | 30952 | 6: 37305466 |
| 30953 | 6: 37305470 | 30954 | 6: 37305490 | 30955 | 6: 37305557 | 30956 | 6: 37306944 | 30957 | 6: 37306975 |
| 30958 | 6: 37307238 | 30961 | 6: 37310693 | 30962 | 6: 37311375 | 30963 | 6: 37311766 | 30964 | 6: 37311816 |
| 30965 | 6: 37311980 | 30966 | 6: 37312071 | 30967 | 6: 37312238 | 30968 | 6: 37312270 | 30969 | 6: 37312326 |
| 30970 | 6: 37312692 | 30971 | 6: 37313215 | 30972 | 6: 37313503 | 30973 | 6: 37313663 | 30974 | 6: 37313742 |
| 30975 | 6: 37313954 | 30976 | 6: 37314022 | 30977 | 6: 37314183 | 30978 | 6: 37314291 | 30979 | 6: 37315223 |
| 30980 | 6: 37315480 | 30981 | 6: 37315752 | 30982 | 6: 37315774 | 30983 | 6: 37316117 | 30984 | 6: 37316265 |
| 30985 | 6: 37316471 | 30986 | 6: 37316594 | 30987 | 6: 37316659 | 30988 | 6: 37316928 | 30989 | 6: 37317087 |
| 30990 | 6: 37317153 | 30991 | 6: 37317167 | 30992 | 6: 37317341 | 30993 | 6: 37317695 | 30994 | 6: 37318285 |
| 30995 | 6: 37318305 | 30996 | 6: 37318658 | 30997 | 6: 37318743 | 30998 | 6: 37318847 | 30999 | 6: 37319284 |
| 31000 | 6: 37319902 | 31001 | 6: 37320093 | 31002 | 6: 37321012 | 31003 | 6: 37321049 | 31004 | 6: 37321441 |
| 31005 | 6: 37321485 | 31006 | 6: 37322361 | 31007 | 6: 37322770 | 31008 | 6: 37323838 | 31009 | 6: 37323843 |
| 31010 | 6: 37324718 | 31011 | 6: 37325097 | 31012 | 6: 37325189 | 31013 | 6: 37326965 | 31014 | 6: 37327393 |
| 31015 | 6: 37327644 | 31016 | 6: 37328014 | 31017 | 6: 37328069 | 31018 | 6: 37328528 | 31019 | 6: 37328898 |
| 31020 | 6: 37329042 | 31021 | 6: 37329061 | 31022 | 6: 37329319 | 31023 | 6: 37329413 | 31024 | 6: 37329526 |
| 31025 | 6: 37329583 | 31027 | 6: 37329948 | 31028 | 6: 37330186 | 31029 | 6: 37330198 | 31031 | 6: 37330365 |
| 31034 | 6: 37330605 | 31035 | 6: 37330677 | 31036 | 6: 37331712 | 31037 | 6: 37331772 | 31038 | 6: 37331921 |
| 31039 | 6: 37332914 | 31040 | 6: 37333089 | 31041 | 6: 37333257 | 31042 | 6: 37333646 | 31043 | 6: 37335096 |
| 31044 | 6: 37335574 | 31045 | 6: 37335884 | 31047 | 6: 37336176 | 31048 | 6: 37336277 | 31049 | 6: 37336431 |
| 31050 | 6: 37337044 | 31051 | 6: 37337343 | 31053 | 6: 37337910 | 31054 | 6: 37340194 | 31055 | 6: 37340475 |
| 31056 | 6: 37340937 | 31057 | 6: 37341535 | 31058 | 6: 37342568 | 31059 | 6: 37342705 | 31060 | 6: 37343364 |
| 31061 | 6: 37343379 | 31062 | 6: 37344246 | 31063 | 6: 37344303 | 31064 | 6: 37344897 | 31065 | 6: 37345259 |
| 31066 | 6: 37345508 | 31067 | 6: 37345931 | 31068 | 6: 37346124 | 31069 | 6: 37346442 | 31070 | 6: 37347130 |
| 31071 | 6: 37349144 | 31072 | 6: 37350316 | 31073 | 6: 37350368 | 31074 | 6: 37351499 | 31075 | 6: 37351767 |
| 31076 | 6: 37352145 | 31077 | 6: 37352282 | 31078 | 6: 37352820 | 31079 | 6: 37353288 | 31080 | 6: 37354945 |
| 31081 | 6: 37355132 | 31082 | 6: 37355369 | 31083 | 6: 37356067 | 31084 | 6: 37357055 | 31085 | 6: 37358828 |
| 31086 | 6: 37359765 | 31087 | 6: 37360499 | 31088 | 6: 37360685 | 31089 | 6: 37361449 | 31090 | 6: 37361578 |
| 31092 | 6: 37363842 | 31093 | 6: 37365265 | 31094 | 6: 37365744 | 31095 | 6: 37366868 | 31096 | 6: 37368371 |
| 31097 | 6: 37368652 | 31098 | 6: 37368670 | 31100 | 6: 37369380 | 31101 | 6: 37370421 | 31103 | 6: 37373261 |
| 31104 | 6: 37373314 | 31105 | 6: 37374314 | 31106 | 6: 37374718 | 31107 | 6: 37376009 | 31109 | 6: 37377606 |
| 31110 | 6: 37377857 | 31112 | 6: 37378824 | 31114 | 6: 37379507 | 31115 | 6: 37379693 | 31116 | 6: 37379729 |
| 31117 | 6: 37379796 | 31118 | 6: 37380161 | 31119 | 6: 37380671 | 31120 | 6: 37381106 | 31121 | 6: 37381616 |
| 31125 | 6: 37382177 | 31126 | 6: 37383284 | 31128 | 6: 37384356 | 31130 | 6: 37390341 | 31131 | 6: 37390788 |
| 31132 | 6: 37391198 | 31133 | 6: 37391915 | 31134 | 6: 37392465 | 31135 | 6: 37392540 | 31136 | 6: 37392957 |
| 31137 | 6: 37393564 | 31138 | 6: 37393949 | 31140 | 6: 37395313 | 31141 | 6: 37395750 | 31142 | 6: 37395838 |
| 31143 | 6: 37396789 | 31144 | 6: 37398199 | 31146 | 6: 37399852 | 31147 | 6: 37400741 | 31148 | 6: 37400800 |
| 31149 | 6: 37402511 | 31150 | 6: 37402964 | 31152 | 6: 37403224 | 31153 | 6: 37406239 | 31154 | 6: 37406485 |
| 31155 | 6: 37406729 | 31156 | 6: 37407653 | 31157 | 6: 37412062 | 31158 | 6: 37412935 | 31159 | 6: 37414352 |
| 31160 | 6: 37414365 | 31161 | 6: 37414416 | 31162 | 6: 37415367 | 31164 | 6: 37416649 | 31165 | 6: 37417046 |
| 31167 | 6: 37418164 | 31168 | 6: 37418421 | 31170 | 6: 37418449 | 31171 | 6: 37420252 | 31173 | 6: 37423505 |
| 31174 | 6: 37424248 | 31175 | 6: 37425030 | 31176 | 6: 37429071 | 31177 | 6: 37429595 | 31180 | 6: 37431101 |
| 31181 | 6: 37432458 | 31182 | 6: 37432854 | 31183 | 6: 37433502 | 31184 | 6: 37433926 | 31185 | 6: 37434244 |
| 31186 | 6: 37435861 | 31187 | 6: 37436146 | 31188 | 6: 37436347 | 31189 | 6: 37436731 | 31190 | 6: 37437130 |
| 31191 | 6: 37437538 | 31193 | 6: 37438248 | 31194 | 6: 37438530 | 31195 | 6: 37438575 | 31196 | 6: 37438599 |
| 31197 | 6: 37438993 | 31198 | 6: 37439190 | 31200 | 6: 37440955 | 31201 | 6: 37441274 | 31202 | 6: 37441899 |
| 31203 | 6: 37442320 | 31204 | 6: 37444900 | 31205 | 6: 37446552 | 31206 | 6: 37448497 | 31207 | 6: 37449358 |
| 31208 | 6: 37449890 | 31209 | 6: 37450921 | 31210 | 6: 37453215 | 31211 | 6: 37456193 | 31213 | 6: 37461999 |
| 31214 | 6: 37463282 | 31215 | 6: 37463709 | 31217 | 6: 37466194 | 31218 | 6: 37473453 | 31219 | 6: 37477629 |
| 31220 | 6: 37479269 | 31221 | 6: 37480277 | 31223 | 6: 37483537 | 31224 | 6: 37489614 | 31226 | 6: 37493760 |
| 31227 | 6: 37495347 | 31228 | 6: 37500943 | 31230 | 6: 37505093 | 31231 | 6: 37508184 | 31232 | 6: 37511610 |
| 31233 | 6: 37512672 | 31234 | 6: 37512811 | 31235 | 6: 37513645 | 31236 | 6: 37520035 | 31237 | 6: 37521235 |
| 31238 | 6: 37522190 | 31239 | 6: 37522580 | 31240 | 6: 37526812 | 31241 | 6: 37527163 | 31242 | 6: 37529800 |
| 31244 | 6: 37543134 | 31245 | 6: 37547826 | 31247 | 6: 37559117 | 31250 | 6: 37564896 | 31251 | 6: 37566268 |
| 31253 | 6: 37566504 | 31255 | 6: 37573615 | 31257 | 6: 37578899 | 31258 | 6: 37579956 | 31259 | 6: 37580857 |
| 31260 | 6: 37585527 | 31261 | 6: 37587677 | 31264 | 6: 37591326 | 31266 | 6: 37592943 | 31267 | 6: 37595517 |
| 31269 | 6: 37599145 | 31270 | 6: 37599699 | 31271 | 6: 37602013 | 31272 | 6: 37604468 | 31273 | 6: 37606625 |
| 31274 | 6: 37607139 | 31275 | 6: 37608496 | 31276 | 6: 37608504 | 31277 | 6: 37614039 | 31278 | 6: 37614179 |
| 31279 | 6: 37617530 | 31280 | 6: 37624003 | 31281 | 6: 37625923 | 31282 | 6: 37627776 | 31283 | 6: 37633434 |
| 31284 | 6: 37636883 | 31285 | 6: 37636900 | 31286 | 6: 37637484 | 31290 | 6: 37641448 | 31293 | 6: 37644691 |
| 31294 | 6: 37644915 | 31295 | 6: 37645671 | 31296 | 6: 37646000 | 31297 | 6: 37646499 | 31298 | 6: 37646597 |
| 31299 | 6: 37652729 | 31300 | 6: 37658289 | 31302 | 6: 37664673 | 31303 | 6: 37666279 | 31304 | 6: 37669453 |
| 31305 | 6: 37674317 | 31306 | 6: 37675400 | 31307 | 6: 37679238 | 31308 | 6: 37681968 | 31309 | 6: 37683699 |
| 31310 | 6: 37687844 | 31312 | 6: 37690283 | 31313 | 6: 37690852 | 31314 | 6: 37696619 | 31315 | 6: 37697757 |
| 31316 | 6: 37698657 | 31317 | 6: 37699059 | 31318 | 6: 37703461 | 31319 | 6: 37704637 | 31320 | 6: 37706431 |
| 31321 | 6: 37706528 | 31322 | 6: 37708048 | 31323 | 6: 37708742 | 31324 | 6: 37708953 | 31325 | 6: 37709524 |
| 31327 | 6: 37712519 | 31328 | 6: 37712749 | 31329 | 6: 37713097 | 31330 | 6: 37713686 | 31331 | 6: 37713889 |
| 31332 | 6: 37719635 | 31333 | 6: 37719804 | 31334 | 6: 37720524 | 31335 | 6: 37727280 | 31336 | 6: 37728142 |
| 31337 | 6: 37731195 | 31338 | 6: 37736960 | 31339 | 6: 37738532 | 31340 | 6: 37740667 | 31341 | 6: 37741893 |
| 31342 | 6: 37742839 | 31343 | 6: 37743712 | 31344 | 6: 37749840 | 31345 | 6: 37749955 | 31346 | 6: 37751452 |
| 31347 | 6: 37752740 | 31348 | 6: 37760883 | 31349 | 6: 37772675 | 31350 | 6: 37773321 | 31351 | 6: 37773355 |
| 31352 | 6: 37774136 | 31354 | 6: 37775491 | 31355 | 6: 37775624 | 31356 | 6: 37775822 | 31357 | 6: 37776401 |
| 31358 | 6: 37776428 | 31359 | 6: 37776454 | 31360 | 6: 37776464 | 31362 | 6: 37777056 | 31363 | 6: 37777185 |
| 31364 | 6: 37778681 | 31365 | 6: 37778924 | 31367 | 6: 37797233 | 31368 | 6: 37802327 | 31369 | 6: 37803937 |
| 31370 | 6: 37803941 | 31372 | 6: 37804729 | 31373 | 6: 37805097 | 31374 | 6: 37806070 | 31376 | 6: 37809966 |
| 31377 | 6: 37810505 | 31378 | 6: 37810779 | 31379 | 6: 37811541 | 31380 | 6: 37811600 | 31381 | 6: 37811808 |
| 31382 | 6: 37811947 | 31383 | 6: 37812109 | 31384 | 6: 37812521 | 31385 | 6: 37813026 | 31386 | 6: 37813294 |
| 31387 | 6: 37813424 | 31388 | 6: 37813572 | 31389 | 6: 37813638 | 31390 | 6: 37813728 | 31391 | 6: 37813964 |
| 31392 | 6: 37814265 | 31393 | 6: 37814928 | 31394 | 6: 37815457 | 31395 | 6: 37815489 | 31396 | 6: 37815615 |
| 31397 | 6: 37815806 | 31398 | 6: 37815918 | 3139 | 6: 37816011 | 31400 | 6: 37816192 | 31401 | 6: 37816231 |
| 31402 | 6: 37816300 | 31403 | 6: 37817310 | 31405 | 6: 37817589 | 31406 | 6: 37817855 | 31407 | 6: 37817875 |
| 31408 | 6: 37817910 | 31409 | 6: 37818107 | 31410 | 6: 37818111 | 31411 | 6: 37818195 | 31412 | 6: 37818372 |
| 31413 | 6: 37819224 | 31414 | 6: 37821523 | 31415 | 6: 37821595 | 31416 | 6: 37821964 | 31417 | 6: 37830099 |
| 31418 | 6: 37831626 | 31419 | 6: 37833439 | 31420 | 6: 37838622 | 31421 | 6: 37842392 | 31422 | 6: 37895331 |
| 31423 | 6: 37902755 | 31424 | 6: 37905296 | 31425 | 6: 37905467 | 31426 | 6: 37907357 | 31427 | 6: 37908478 |
| 31428 | 6: 37908507 | 31429 | 6: 37908679 | 31430 | 6: 37908861 | 31431 | 6: 37908962 | 31432 | 6: 37909143 |
| 31433 | 6: 37910276 | 31434 | 6: 37910464 | 31435 | 6: 37910478 | 31436 | 6: 37910866 | 31437 | 6: 37911814 |
| 31438 | 6: 37912116 | 31439 | 6: 37913125 | 31440 | 6: 37913652 | 31441 | 6: 37913727 | 31442 | 6: 37913922 |
| 31443 | 6: 37913940 | 31444 | 6: 37914439 | 31446 | 6: 37914513 | 31447 | 6: 37914730 | 31448 | 6: 37914816 |
| 31449 | 6: 37914822 | 31450 | 6: 37914832 | 31451 | 6: 37915050 | 31452 | 6: 37915308 | 31453 | 6: 37916419 |
| 31454 | 6: 37917366 | 31455 | 6: 37917421 | 31456 | 6: 37917796 | 31457 | 6: 37918546 | 31458 | 6: 37918824 |
| 31459 | 6: 37919045 | 31460 | 6: 37919095 | 31461 | 6: 37919120 | 31462 | 6: 37919520 | 31463 | 6: 37919631 |
| 31464 | 6: 37919661 | 31465 | 6: 37919853 | 31466 | 6: 37919862 | 31467 | 6: 37920305 | 31468 | 6: 37920538 |
| 31469 | 6: 37920551 | 31470 | 6: 37920666 | 31471 | 6: 37921510 | 31472 | 6: 37921603 | 31474 | 6: 37921724 |
| 31476 | 6: 37926600 | 31477 | 6: 37926960 | 31478 | 6: 37928156 | 31479 | 6: 37928479 | 31480 | 6: 37929065 |
| 31481 | 6: 37929104 | 31482 | 6: 37929184 | 31483 | 6: 37929689 | 31484 | 6: 37929786 | 31485 | 6: 37930046 |
| 31487 | 6: 37930293 | 31488 | 6: 37930672 | 31489 | 6: 37930781 | 31490 | 6: 37930868 | 31491 | 6: 37930889 |
| 31492 | 6: 37931089 | 31493 | 6: 37931216 | 31494 | 6: 37931298 | 31495 | 6: 37931621 | 31496 | 6: 37931688 |
| 31497 | 6: 37931726 | 31498 | 6: 37931903 | 31499 | 6: 37932170 | 31500 | 6: 37932197 | 31501 | 6: 37932241 |
| 31502 | 6: 37932371 | 31503 | 6: 37932377 | 31504 | 6: 37932412 | 31505 | 6: 37932434 | 31506 | 6: 37932563 |
| 31508 | 6: 37932772 | 31509 | 6: 37932778 | 31510 | 6: 37932960 | 31511 | 6: 37933250 | 31512 | 6: 37933352 |
| 31513 | 6: 37933965 | 31514 | 6: 37934068 | 31515 | 6: 37934272 | 31516 | 6: 37934993 | 31517 | 6: 37935069 |
| 31518 | 6: 37935108 | 31519 | 6: 37935168 | 31520 | 6: 37935527 | 31521 | 6: 37935577 | 31522 | 6: 37935748 |
| 31523 | 6: 37936044 | 31524 | 6: 37936087 | 31525 | 6: 37936692 | 31526 | 6: 37938294 | 31529 | 6: 37942096 |
| 31530 | 6: 37942170 | 31531 | 6: 37942290 | 31532 | 6: 37942944 | 31533 | 6: 37943635 | 31534 | 6: 37943860 |
| 31536 | 6: 37944478 | 31537 | 6: 37944482 | 31538 | 6: 37944496 | 31539 | 6: 37944573 | 31540 | 6: 37944631 |
| 31541 | 6: 37944681 | 31542 | 6: 37944704 | 31543 | 6: 37945333 | 31544 | 6: 37945482 | 31545 | 6: 37945820 |
| 31546 | 6: 37945849 | 31547 | 6: 37946012 | 31548 | 6: 37946384 | 31549 | 6: 37953254 | 31550 | 6: 37953301 |
| 31551 | 6: 37953591 | 31552 | 6: 37954699 | 31553 | 6: 37954789 | 31554 | 6: 37954873 | 31555 | 6: 37955372 |
| 31556 | 6: 37956368 | 31557 | 6: 37956390 | 31558 | 6: 37959360 | 31559 | 6: 37960313 | 31560 | 6: 37961491 |
| 31561 | 6: 37961553 | 31562 | 6: 37962093 | 31563 | 6: 37962887 | 31564 | 6: 37963346 | 31565 | 6: 37963525 |
| 31566 | 6: 37965259 | 31567 | 6: 37965489 | 31568 | 6: 37966382 | 31569 | 6: 37968595 | 31570 | 6: 37970172 |
| 31571 | 6: 37970860 | 31572 | 6: 37971218 | 31573 | 6: 37971544 | 31574 | 6: 37971810 | 31575 | 6: 37971997 |
| 31576 | 6: 37972173 | 31577 | 6: 37972335 | 31578 | 6: 37972414 | 31579 | 6: 37972642 | 31580 | 6: 37972906 |
| 31581 | 6: 37973011 | 31582 | 6: 37973508 | 31583 | 6: 37973804 | 31584 | 6: 37974302 | 31586 | 6: 37974407 |
| 31587 | 6: 37974974 | 31588 | 6: 37976818 | 31589 | 6: 37978037 | 31590 | 6: 37978995 | 31591 | 6: 37979083 |
| 31592 | 6: 37981093 | 31593 | 6: 37981125 | 31594 | 6: 37983153 | 31595 | 6: 37984808 | 31596 | 6: 37984948 |
| 31597 | 6: 37985243 | 31598 | 6: 37987337 | 31599 | 6: 37987503 | 31600 | 6: 37989831 | 31601 | 6: 37990674 |
| 31602 | 6: 37990808 | 31603 | 6: 37991751 | 31604 | 6: 37992856 | 31605 | 6: 37993125 | 31606 | 6: 37993517 |
| 31607 | 6: 37993754 | 31608 | 6: 37994046 | 31609 | 6: 37996786 | 31610 | 6: 37996859 | 31611 | 6: 37997160 |
| 31612 | 6: 37997516 | 31613 | 6: 37998187 | 31614 | 6: 37998452 | 31615 | 6: 37998592 | 31616 | 6: 37998656 |
| 31617 | 6: 37998696 | 31618 | 6: 37999740 | 31619 | 6: 37999825 | 31620 | 6: 37999842 | 31621 | 6: 38000343 |
| 31622 | 6: 38000353 | 31623 | 6: 38001392 | 31624 | 6: 38001622 | 31625 | 6: 38002117 | 31626 | 6: 38002596 |
| 31627 | 6: 38004124 | 31628 | 6: 38004325 | 31629 | 6: 38005805 | 31630 | 6: 38007975 | 31631 | 6: 38014109 |
| 31632 | 6: 38016521 | 31633 | 6: 38017827 | 31634 | 6: 38017866 | 31635 | 6: 38021694 | 31636 | 6: 38022226 |
| 31637 | 6: 38022305 | 31638 | 6: 38023218 | 31639 | 6: 38023327 | 31640 | 6: 38023545 | 31641 | 6: 38023613 |
| 31642 | 6: 38023673 | 31643 | 6: 38023878 | 31644 | 6: 38024011 | 31645 | 6: 38024702 | 31646 | 6: 38024768 |
| 31647 | 6: 38025411 | 31648 | 6: 38025684 | 31649 | 6: 38025734 | 31650 | 6: 38026545 | 31651 | 6: 38027078 |
| 31652 | 6: 38027106 | 31653 | 6: 38027129 | 31654 | 6: 38031018 | 31655 | 6: 38032275 | 31656 | 6: 38033896 |
| 31658 | 6: 38035001 | 31659 | 6: 38035414 | 31660 | 6: 38035460 | 31661 | 6: 38035561 | 31662 | 6: 38035568 |
| 31663 | 6: 38035625 | 31664 | 6: 38035705 | 31665 | 6: 38036061 | 31666 | 6: 38036351 | 31667 | 6: 38036588 |
| 31668 | 6: 38036594 | 31669 | 6: 38036635 | 31670 | 6: 38036660 | 31671 | 6: 38036882 | 31672 | 6: 38037099 |
| 31673 | 6: 38037535 | 31674 | 6: 38037724 | 31675 | 6: 38037768 | 31676 | 6: 38038091 | 31677 | 6: 38038189 |
| 31678 | 6: 38038404 | 31679 | 6: 38038608 | 31680 | 6: 38039088 | 31681 | 6: 38039492 | 31682 | 6: 38040456 |
| 31683 | 6: 38040488 | 31684 | 6: 38040529 | 31685 | 6: 38040769 | 31686 | 6: 38040934 | 31687 | 6: 38041234 |
| 31688 | 6: 38041593 | 31689 | 6: 38041633 | 31690 | 6: 38041715 | 31691 | 6: 38042205 | 31692 | 6: 38042337 |
| 31693 | 6: 38043977 | 31694 | 6: 38044618 | 31695 | 6: 38044876 | 31696 | 6: 38044898 | 31697 | 6: 38045727 |
| 31698 | 6: 38046428 | 31699 | 6: 38048735 | 31700 | 6: 38051406 | 31701 | 6: 38051509 | 31702 | 6: 38052049 |
| 31703 | 6: 38052245 | 31704 | 6: 38054733 | 31706 | 6: 38063490 | 31707 | 6: 38064317 | 31708 | 6: 38065703 |
| 31709 | 6: 38066309 | 31710 | 6: 38067289 | 31711 | 6: 38067376 | 31712 | 6: 38067855 | 31713 | 6: 38068311 |
| 31714 | 6: 38068956 | 31715 | 6: 38069662 | 31716 | 6: 38070361 | 31717 | 6: 38070496 | 31718 | 6: 38070882 |
| 31719 | 6: 38071318 | 31720 | 6: 38071414 | 31721 | 6: 38071547 | 31722 | 6: 38073914 | 31723 | 6: 38076161 |
| 31724 | 6: 38076905 | 31725 | 6: 38077237 | 31726 | 6: 38077323 | 31728 | 6: 38078774 | 31729 | 6: 38079456 |
| 31730 | 6: 38079499 | 31731 | 6: 38082355 | 31732 | 6: 38085803 | 31733 | 6: 38087237 | 31734 | 6: 38087636 |
| 31735 | 6: 38089862 | 31736 | 6: 38094533 | 31737 | 6: 38097987 | 31739 | 6: 38107020 | 31740 | 6: 38108493 |
| 31742 | 6: 38114043 | 31743 | 6: 38114047 | 31744 | 6: 38114717 | 31745 | 6: 38114803 | 31746 | 6: 38114808 |
| 31747 | 6: 38115023 | 31748 | 6: 38118015 | 31749 | 6: 38122903 | 31750 | 6: 38123039 | 31751 | 6: 38127700 |
| 31752 | 6: 38131158 | 31753 | 6: 38132133 | 31754 | 6: 38132688 | 31755 | 6: 38135706 | 31756 | 6: 38137640 |
| 31757 | 6: 38137799 | 31758 | 6: 38138017 | 31759 | 6: 38138089 | 31760 | 6: 38139495 | 31761 | 6: 38139563 |
| 31762 | 6: 38139762 | 31763 | 6: 38140108 | 31764 | 6: 38140123 | 31765 | 6: 38140536 | 31766 | 6: 38140681 |
| 31767 | 6: 38141284 | 31768 | 6: 38141304 | 31769 | 6: 38141629 | 31770 | 6: 38141696 | 31771 | 6: 38141833 |
| 31772 | 6: 38142148 | 31773 | 6: 38142807 | 31774 | 6: 38142835 | 31775 | 6: 38142840 | 31776 | 6: 38144120 |
| 31777 | 6: 38144195 | 31778 | 6: 38144291 | 31779 | 6: 38144317 | 31780 | 6: 38144587 | 31781 | 6: 38156378 |
| 31782 | 6: 38156469 | 31783 | 6: 38156591 | 31784 | 6: 38157637 | 31785 | 6: 38157684 | 31786 | 6: 38158107 |
| 31787 | 6: 38158113 | 31791 | 6: 38199691 | 31792 | 6: 38202499 | 31793 | 6: 38211198 | 31795 | 6: 38227779 |
| 31797 | 6: 38237566 | 31798 | 6: 38237695 | 31799 | 6: 38247802 | 31800 | 6: 38252776 | 31805 | 6: 38278657 |
| 31806 | 6: 38278666 | 31807 | 6: 38278782 | 31808 | 6: 38279030 | 31809 | 6: 38281274 | 31814 | 6: 38318628 |
| 31817 | 6: 38375856 | 31820 | 6: 38396789 | 31823 | 6: 38409609 | 31825 | 6: 38433243 | 31827 | 6: 38499580 |
| 31828 | 6: 38500871 | 31829 | 6: 38506361 | 31830 | 6: 38508464 | 31832 | 6: 38519250 | 31833 | 6: 38525790 |
| 31834 | 6: 38535911 | 31835 | 6: 38540318 | 31838 | 6: 38541639 | 31840 | 6: 38542150 | 31844 | 6: 38543990 |
| 31848 | 6: 38546646 | 31849 | 6: 38548060 | 31850 | 6: 38548198 | 31854 | 6: 38549044 | 31856 | 6: 38549206 |
| 31858 | 6: 38549703 | 31859 | 6: 38550162 | 31869 | 6: 38553181 | 31881 | 6: 38556220 | 31895 | 6: 38562229 |
| 31896 | 6: 38563063 | 31898 | 6: 38565084 | 31899 | 6: 38566242 | 31900 | 6: 38570744 | 31901 | 6: 38575762 |
| 31903 | 6: 38584420 | 31904 | 6: 38584686 | 31905 | 6: 38588303 | 31906 | 6: 38599441 | 31907 | 6: 38600028 |
| 31908 | 6: 38600235 | 31909 | 6: 38602696 | 31910 | 6: 38617268 | 31911 | 6: 38617890 | 31912 | 6: 38618593 |
| 31913 | 6: 38619398 | 31914 | 6: 38619754 | 31915 | 6: 38619795 | 31916 | 6: 38620014 | 31917 | 6: 38620218 |
| 31918 | 6: 38621930 | 31919 | 6: 38624000 | 31920 | 6: 38624126 | 31921 | 6: 38644939 | 31923 | 6: 38695951 |
| 31924 | 6: 38697400 | 31928 | 6: 38789984 | 31929 | 6: 38798194 | 31933 | 6: 38854750 | 31942 | 6: 38907049 |
| 31943 | 6: 38918524 | 31945 | 6: 38919029 | 31946 | 6: 38929469 | 31947 | 6: 38933534 | 31948 | 6: 38934821 |
| 31950 | 6: 38963229 | 31951 | 6: 38973635 | 31952 | 6: 38973995 | 31953 | 6: 38980873 | 31954 | 6: 39030259 |
| 31956 | 6: 39082331 | 31957 | 6: 39109137 | 31962 | 6: 39181262 | 31966 | 6: 39254458 | 31968 | 6: 39280700 |
| 31974 | 6: 39437165 | 31977 | 6: 39518939 | 31978 | 6: 39532147 | 31979 | 6: 39559751 | 31982 | 6: 39574982 |
| 31984 | 6: 39595691 | 31985 | 6: 39595731 | 31988 | 6: 39652421 | 31992 | 6: 39721697 | 31994 | 6: 39730068 |
| 31995 | 6: 39733610 | 31996 | 6: 39737879 | 31998 | 6: 39755798 | 31999 | 6: 39756006 | 32002 | 6: 39760650 |
| 32003 | 6: 39772834 | 32004 | 6: 39772963 | 32005 | 6: 39773133 | 32007 | 6: 39797151 | 32009 | 6: 39825526 |
| 32011 | 6: 39858450 | 32012 | 6: 39874821 | 32013 | 6: 39885647 | 32026 | 6: 40072649 | 32030 | 6: 40130949 |
| 32032 | 6: 40203593 | 32035 | 6: 40239109 | 32036 | 6: 40248052 | 32042 | 6: 40338033 | 32047 | 6: 40400197 |
| 32048 | 6: 40432849 | 32049 | 6: 40456680 | 32052 | 6: 40565574 | 32055 | 6: 40599155 | 32058 | 6: 40636686 |
| 32059 | 6: 40662051 | 32060 | 6: 40672232 | 32061 | 6: 40689118 | 32062 | 6: 40696320 | 32085 | 6: 40731922 |
| 32089 | 6: 40758035 | 32093 | 6: 40780850 | 32094 | 6: 40808612 | 32098 | 6: 40836283 | 32102 | 6: 40911873 |
| 32103 | 6: 40920367 | 32104 | 6: 40921708 | 32105 | 6: 40933517 | 32106 | 6: 40935693 | 32107 | 6: 40935713 |
| 32108 | 6: 40935857 | 32109 | 6: 40935890 | 32110 | 6: 40936031 | 32111 | 6: 40936207 | 32112 | 6: 40936326 |
| 32113 | 6: 40936831 | 32114 | 6: 40936837 | 32115 | 6: 40937031 | 32116 | 6: 40937441 | 32117 | 6: 40942588 |
| 32120 | 6: 40984868 | 32121 | 6: 40988645 | 32122 | 6: 40988913 | 32123 | 6: 40999090 | 32124 | 6: 41003445 |
| 32125 | 6: 41004894 | 32126 | 6: 41029896 | 32127 | 6: 41033792 | 32128 | 6: 41049370 | 32129 | 6: 41049496 |
| 32130 | 6: 41049859 | 32131 | 6: 41049914 | 32132 | 6: 41050127 | 32133 | 6: 41050140 | 32135 | 6: 41050862 |
| 32136 | 6: 41051120 | 32137 | 6: 41053625 | 32138 | 6: 41061635 | 32139 | 6: 41073550 | 32140 | 6: 41079462 |
| 32142 | 6: 41088062 | 32143 | 6: 41090326 | 32144 | 6: 41096500 | 32145 | 6: 41107449 | 32146 | 6: 41107836 |
| 32147 | 6: 41108192 | 32148 | 6: 41108818 | 32149 | 6: 41108912 | 32150 | 6: 41108926 | 32151 | 6: 41109137 |
| 32152 | 6: 41109217 | 32153 | 6: 41109350 | 32155 | 6: 41150218 | 32156 | 6: 41169318 | 32157 | 6: 41170432 |
| 32158 | 6: 41173967 | 32161 | 6: 41185741 | 32162 | 6: 41204988 | 32169 | 6: 41306166 | 32170 | 6: 41306273 |
| 32171 | 6: 41306284 | 32172 | 6: 41306424 | 32173 | 6: 41308067 | 32174 | 6: 41331258 | 32179 | 6: 41577847 |
| 32180 | 6: 41592300 | 32181 | 6: 41677493 | 32188 | 6: 41868518 | 32195 | 6: 42004652 | 32203 | 6: 42037316 |
| 32209 | 6: 42163671 | 32216 | 6: 42441818 | 32220 | 6: 42507082 | 32221 | 6: 42510330 | 32227 | 6: 42559603 |
| 32229 | 6: 42665842 | 32230 | 6: 42672683 | 32231 | 6: 42722868 | 32232 | 6: 42724776 | 32233 | 6: 42728138 |
| 32234 | 6: 42741244 | 32235 | 6: 42741447 | 32236 | 6: 42758331 | 32241 | 6: 42837669 | 32263 | 6: 42974326 |
| 32284 | 6: 43317249 | 32296 | 6: 43494887 | 32298 | 6: 43544441 | 32303 | 6: 43601154 | 32309 | 6: 43679917 |
| 32318 | 6: 43822090 | 32319 | 6: 43822403 | 32320 | 6: 43829217 | 32323 | 6: 43886543 | 32326 | 6: 43944737 |
| 32330 | 6: 43983354 | 32331 | 6: 44003666 | 32332 | 6: 44026498 | 32336 | 6: 44072335 | 32339 | 6: 44149286 |
| 32343 | 6: 44276184 | 32360 | 6: 44628911 | 32362 | 6: 44678042 | 32363 | 6: 44685469 | 32364 | 6: 44686809 |
| 32365 | 6: 44689379 | 32366 | 6: 44690759 | 32368 | 6: 44694506 | 32369 | 6: 44694605 | 32370 | 6: 44695255 |
| 32371 | 6: 44695433 | 32372 | 6: 44695563 | 32373 | 6: 44695784 | 32374 | 6: 44695872 | 32375 | 6: 44695978 |
| 32376 | 6: 44695987 | 32377 | 6: 44696022 | 32378 | 6: 44696578 | 32379 | 6: 44696796 | 32380 | 6: 44696931 |
| 32381 | 6: 44696955 | 32382 | 6: 44698160 | 32383 | 6: 44704061 | 32384 | 6: 44704410 | 32385 | 6: 44705240 |
| 32386 | 6: 44706199 | 32387 | 6: 44706279 | 32388 | 6: 44709016 | 32389 | 6: 44709754 | 32390 | 6: 44710003 |
| 32391 | 6: 44710237 | 32392 | 6: 44710415 | 32393 | 6: 44710515 | 32394 | 6: 44711099 | 32395 | 6: 44712295 |
| 32396 | 6: 44735374 | 32397 | 6: 44736099 | 32398 | 6: 44739055 | 32399 | 6: 44742360 | 32400 | 6: 44747933 |
| 32401 | 6: 44748956 | 32403 | 6: 44775326 | 32404 | 6: 44777357 | 32405 | 6: 44784742 | 32406 | 6: 44787261 |
| 32407 | 6: 44791513 | 32408 | 6: 44791744 | 32409 | 6: 44797875 | 32410 | 6: 44809081 | 32411 | 6: 44810108 |
| 32412 | 6: 44818322 | 32413 | 6: 44819710 | 32414 | 6: 44820115 | 32415 | 6: 44823097 | 32416 | 6: 44825630 |
| 32418 | 6: 44828213 | 32419 | 6: 44831889 | 32420 | 6: 44857705 | 32421 | 6: 44864378 | 32422 | 6: 44873514 |
| 32423 | 6: 44873843 | 32424 | 6: 44874186 | 32425 | 6: 44877670 | 32426 | 6: 44883344 | 32427 | 6: 44893464 |
| 32428 | 6: 44904257 | 32429 | 6: 44905170 | 32430 | 6: 44909660 | 32431 | 6: 44914656 | 32432 | 6: 44920744 |
| 32433 | 6: 44931581 | 32434 | 6: 44934298 | 32435 | 6: 44934689 | 32437 | 6: 44983137 | 32439 | 6: 45018531 |
| 32440 | 6: 45018882 | 32441 | 6: 45029390 | 32442 | 6: 45029425 | 32443 | 6: 45029626 | 32444 | 6: 45030486 |
| 32445 | 6: 45030599 | 32446 | 6: 45030709 | 32447 | 6: 45030774 | 32448 | 6: 45030789 | 32449 | 6: 45031327 |
| 32450 | 6: 45031721 | 32451 | 6: 45031956 | 32452 | 6: 45032017 | 32453 | 6: 45032153 | 32454 | 6: 45033264 |
| 32455 | 6: 45037315 | 32456 | 6: 45037561 | 32457 | 6: 45037753 | 32458 | 6: 45037798 | 32459 | 6: 45038091 |
| 32460 | 6: 45038647 | 32461 | 6: 45038676 | 32462 | 6: 45038869 | 32463 | 6: 45040756 | 32464 | 6: 45041754 |
| 32465 | 6: 45043489 | 32466 | 6: 45043654 | 32467 | 6: 45043859 | 32468 | 6: 45043984 | 32469 | 6: 45044149 |
| 32470 | 6: 45045596 | 32471 | 6: 45048213 | 32472 | 6: 45048323 | 32473 | 6: 45049586 | 32474 | 6: 45053716 |
| 32475 | 6: 45054332 | 32476 | 6: 45055326 | 32477 | 6: 45057776 | 32478 | 6: 45057805 | 32479 | 6: 45058034 |
| 32480 | 6: 45058273 | 32481 | 6: 45077729 | 32485 | 6: 45135036 | 32487 | 6: 45183436 | 32489 | 6: 45185710 |
| 32490 | 6: 45190122 | 32491 | 6: 45205382 | 32492 | 6: 45213648 | 32497 | 6: 45274484 | 32500 | 6: 45322366 |
| 32502 | 6: 45325845 | 32505 | 6: 45364629 | 32518 | 6: 45583636 | 32519 | 6: 45595528 | 32526 | 6: 45885499 |
| 32569 | 6: 46705772 | 32576 | 6: 46946921 | 32580 | 6: 47016991 | 32586 | 6: 47147481 | 32587 | 6: 47164702 |
| 32591 | 6: 47219584 | 32592 | 6: 47256173 | 32594 | 6: 47317499 | 32596 | 6: 47365064 | 32599 | 6: 47424087 |
| 32602 | 6: 47570425 | 32614 | 6: 47825353 | 32615 | 6: 47843766 | 32616 | 6: 47866852 | 32621 | 6: 47989356 |
| 32623 | 6: 48030364 | 32626 | 6: 48149995 | 32648 | 6: 48893818 | 32652 | 6: 49019297 | 32655 | 6: 49094600 |
| 32662 | 6: 49295440 | 32679 | 6: 49701147 | 32694 | 6: 49848545 | 32696 | 6: 49878456 | 32701 | 6: 50009636 |
| 32703 | 6: 50090470 | 32704 | 6: 50126302 | 32709 | 6: 50252554 | 32716 | 6: 50385741 | 32717 | 6: 50412585 |
| 32719 | 6: 50455132 | 32724 | 6: 50672369 | 32729 | 6: 50779278 | 32731 | 6: 50856440 | 32748 | 6: 51325072 |
| 32750 | 6: 51362285 | 32759 | 6: 51740645 | 32778 | 6: 52250315 | 32780 | 6: 52275948 | 32781 | 6: 52320441 |
| 32782 | 6: 52339267 | 32783 | 6: 52339325 | 32784 | 6: 52347811 | 32785 | 6: 52347899 | 32786 | 6: 52347995 |
| 32787 | 6: 52348073 | 32788 | 6: 52348287 | 32789 | 6: 52348292 | 32790 | 6: 52348643 | 32791 | 6: 52348674 |
| 32792 | 6: 52349023 | 32793 | 6: 52349157 | 32794 | 6: 52349544 | 32795 | 6: 52349562 | 32796 | 6: 52349794 |
| 32797 | 6: 52350003 | 32798 | 6: 52350227 | 32799 | 6: 52350240 | 32800 | 6: 52351181 | 32801 | 6: 52351388 |
| 32802 | 6: 52352446 | 32817 | 6: 52645662 | 32818 | 6: 52671724 | 32825 | 6: 52864424 | 32829 | 6: 52954024 |
| 32831 | 6: 53024244 | 32842 | 6: 53220820 | 32846 | 6: 53285747 | 32847 | 6: 53314886 | 32850 | 6: 53397301 |
| 32853 | 6: 53471919 | 32862 | 6: 53752557 | 32873 | 6: 54004873 | 32874 | 6: 54031927 | 32877 | 6: 54086328 |
| 32879 | 6: 54185052 | 32897 | 6: 54783996 | 32913 | 6: 55303674 | 32920 | 6: 55450495 | 32941 | 6: 55514144 |
| 32976 | 6: 56317455 | 32994 | 6: 56900473 | 33006 | 6: 57179884 | 33012 | 6: 57260380 | 33031 | 6: 57842883 |
| 33035 | 6: 57984344 | 33039 | 6: 58077967 | 33066 | 6: 58746181 | 33077 | 6: 59130010 | 33095 | 6: 59579872 |
| 33097 | 6: 59716766 | 33111 | 6: 59875075 | 33114 | 6: 59907442 | 33122 | 6: 60085027 | 33129 | 6: 60257426 |
| 33137 | 6: 60400323 | 33155 | 6: 60802613 | 33168 | 6: 61185257 | 33176 | 6: 61390096 | 33183 | 6: 61487026 |
| 33204 | 6: 62234468 | 33223 | 6: 62814916 | 33224 | 6: 62862206 | 33225 | 6: 62889960 | 33227 | 6: 62925583 |
| 33238 | 6: 63122811 | 33240 | 6: 63170295 | 33242 | 6: 63209301 | 33244 | 6: 63244734 | 33260 | 6: 63836353 |
| 33307 | 6: 65456807 | 33308 | 6: 65520848 | 33338 | 6: 66507621 | 33350 | 6: 66896326 | 33361 | 6: 67256076 |
| 33376 | 6: 67767947 | 33381 | 6: 67991798 | 33394 | 6: 68331252 | 33397 | 6: 68416278 | 33418 | 6: 68896760 |
| 33422 | 6: 69030825 | 33433 | 6: 69252356 | 33435 | 6: 69346909 | 33444 | 6: 69724839 | 33474 | 6: 70673035 |
| 33478 | 6: 70789469 | 33484 | 6: 70978405 | 33516 | 6: 72022899 | 33530 | 6: 72349944 | 33534 | 6: 72385883 |
| 33535 | 6: 72419275 | 33544 | 6: 72713559 | 33582 | 6: 74091185 | 33584 | 6: 74174913 | 33607 | 6: 75066888 |
| 33608 | 6: 75102649 | 33667 | 6: 76880221 | 33698 | 6: 77967685 | 33734 | 6: 79446058 | 33751 | 6: 80043649 |
| 33764 | 6: 80444445 | 33798 | 6: 81507132 | 33851 | 6: 83098669 | 33875 | 6: 83547735 | 33897 | 6: 83979787 |
| 33921 | 6: 84628765 | 33933 | 6: 85067620 | 33936 | 6: 85092523 | 33946 | 6: 85177598 | 33953 | 6: 85422786 |
| 33964 | 6: 85548797 | 34000 | 6: 86388176 | 34002 | 6: 86478657 | 34055 | 6: 87399244 | 34089 | 6: 88024804 |
| 34126 | 6: 89007570 | 34128 | 6: 89048060 | 34273 | 6: 91629835 | 34306 | 6: 92470502 | 34307 | 6: 92504637 |
| 34410 | 6: 94755352 | 34412 | 6: 94776615 | 34452 | 6: 96090013 | 34453 | 6: 96097237 | 34478 | 6: 96544331 |
| 34596 | 6: 98671599 | 34598 | 6: 98696365 | 34635 | 6: 99753620 | 34640 | 6: 99898377 | 34644 | 6: 100026934 |
| 34702 | 6: 101156203 | 34718 | 6: 101492004 | 34738 | 6: 101904289 | 34742 | 6: 101951985 | 34771 | 6: 102466503 |
| 34774 | 6: 102525990 | 34781 | 6: 102622674 | 34936 | 6: 105596054 | 35002 | 6: 107330690 | 35294 | 6: 113769965 |
| 35299 | 6: 113826996 | 35354 | 6: 114785574 | 35370 | 6: 115107065 | 35390 | 6: 115648265 | 35391 | 6: 115670310 |
| 35405 | 6: 115976356 | 35426 | 6: 116325904 | 35456 | 6: 117124190 | 35476 | 7: 210754 | 35491 | 7: 521674 |
| 35511 | 7: 987326 | 35515 | 7: 1068152 | 35516 | 7: 1068170 | 35638 | 7: 3108200 | 35680 | 7: 3821837 |
| 35729 | 7: 5027580 | 35741 | 7: 5125016 | 35859 | 7: 6346703 | 35871 | 7: 6483779 | 36139 | 7: 13283360 |
| 36142 | 7: 13315974 | 36174 | 7: 13946048 | 36270 | 7: 15409865 | 36316 | 7: 16530067 | 36366 | 7: 17643702 |
| 36378 | 7: 17996900 | 36554 | 7: 21064478 | 36624 | 7: 22566377 | 36628 | 7: 22594727 | 36656 | 7: 23523707 |
| 36685 | 7: 24072015 | 36686 | 7: 24142269 | 36697 | 7: 24434170 | 36698 | 7: 24436989 | 36816 | 7: 26920631 |
| 36863 | 7: 27929188 | 36943 | 7: 29802791 | 36954 | 7: 30036916 | 36957 | 7: 30105269 | 37015 | 7: 31343335 |
| 37063 | 7: 32305922 | 37066 | 7: 32334690 | 37099 | 7: 33229945 | 37111 | 7: 33525039 | 37122 | 7: 33761341 |
| 37149 | 7: 34411840 | 37156 | 7: 34553590 | 37176 | 7: 35028517 | 37430 | 7: 40687768 | 37528 | 7: 43355224 |
| 37570 | 7: 44672168 | 37748 | 7: 48982387 | 37783 | 7: 50794543 | 37816 | 7: 52176483 | 37833 | 7: 52224828 |
| 37838 | 7: 52249785 | 38015 | 7: 56078682 | 38041 | 7: 56774182 | 38048 | 7: 56997351 | 38071 | 7: 57843022 |
| 38075 | 7: 57875535 | 38208 | 7: 60668104 | 38263 | 7: 61497597 | 38359 | 7: 63355823 | 38427 | 7: 65211026 |
| 38428 | 7: 65229182 | 38429 | 7: 65236569 | 38430 | 7: 65246212 | 38431 | 7: 65252605 | 38470 | 7: 66072980 |
| 38560 | 7: 67858059 | 38734 | 7: 72087070 | 38741 | 7: 72254021 | 38780 | 7: 73605674 | 38781 | 7: 73607632 |
| 38782 | 7: 73637074 | 38785 | 7: 73667360 | 38867 | 7: 75759697 | 38875 | 7: 75832195 | 38885 | 7: 76174417 |
| 38922 | 7: 77054436 | 38942 | 7: 77465055 | 38993 | 7: 78445222 | 38999 | 7: 78709632 | 39099 | 7: 80473773 |
| 39124 | 7: 80863489 | 39132 | 7: 81093522 | 39153 | 7: 81316371 | 39179 | 7: 81968933 | 39185 | 7: 82214691 |
| 39191 | 7: 82275842 | 39194 | 7: 82369878 | 39298 | 7: 85032366 | 39351 | 7: 86627973 | 39378 | 7: 87376691 |
| 39402 | 7: 88102064 | 39416 | 7: 88541691 | 39453 | 7: 89335556 | 39457 | 7: 89404622 | 39489 | 7: 90218611 |
| 39512 | 7: 90622467 | 39514 | 7: 90688919 | 39518 | 7: 90701213 | 39520 | 7: 90747139 | 39521 | 7: 90755504 |
| 39522 | 7: 90756120 | 39526 | 7: 90805595 | 39527 | 7: 90807032 | 39528 | 7: 90807192 | 39529 | 7: 90807464 |
| 39530 | 7: 90807690 | 39531 | 7: 90807800 | 39532 | 7: 90807968 | 39533 | 7: 90808002 | 39534 | 7: 90808188 |
| 39535 | 7: 90808858 | 39536 | 7: 90813061 | 39537 | 7: 90813398 | 39539 | 7: 90818194 | 39540 | 7: 90818916 |
| 39541 | 7: 90819613 | 39542 | 7: 90820830 | 39543 | 7: 90821093 | 39544 | 7: 90821542 | 39545 | 7: 90821986 |
| 39546 | 7: 90822179 | 39547 | 7: 90822452 | 39548 | 7: 90822601 | 39549 | 7: 90823320 | 39550 | 7: 90824804 |
| 39551 | 7: 90825013 | 39552 | 7: 90825646 | 39553 | 7: 90825779 | 39554 | 7: 90825887 | 39555 | 7: 90827178 |
| 39557 | 7: 90831014 | 39562 | 7: 90842614 | 39567 | 7: 90851664 | 39571 | 7: 90889577 | 39597 | 7: 91489258 |
| 39602 | 7: 91611232 | 39603 | 7: 91625156 | 39604 | 7: 91625242 | 39605 | 7: 91625323 | 39606 | 7: 91625348 |
| 39609 | 7: 91710576 | 39613 | 7: 91786432 | 39615 | 7: 91796723 | 39616 | 7: 91818525 | 39624 | 7: 91975067 |
| 39702 | 7: 94438004 | 39707 | 7: 94529183 | 39744 | 7: 95454269 | 39752 | 7: 95607084 | 39792 | 7: 96165472 |
| 39942 | 7: 96409276 | 39956 | 7: 96720179 | 39962 | 7: 96807889 | 39981 | 7: 97434118 | 39990 | 7: 97603739 |
| 40255 | 7: 104401635 | 40270 | 7: 104812484 | 40304 | 7: 105679720 | 40323 | 7: 106127282 | 40332 | 7: 106311771 |
| 40335 | 7: 106392644 | 40416 | 7: 108497744 | 40421 | 7: 108592553 | 40429 | 7: 108781177 | 40437 | 7: 108939690 |
| 40523 | 8: 905854 | 40531 | 8: 972589 | 40654 | 8: 3953653 | 40665 | 8: 4218936 | 40702 | 8: 5114851 |
| 40704 | 8: 5164202 | 40769 | 8: 6831018 | 41003 | 8: 12159615 | 41129 | 8: 15805194 | 41141 | 8: 16218371 |
| 41181 | 8: 17034771 | 41209 | 8: 17591195 | 41218 | 8: 17834202 | 41244 | 8: 18677218 | 41417 | 8: 23907022 |
| 41564 | 8: 25653708 | 41594 | 8: 26211730 | 41629 | 8: 27285102 | 41727 | 8: 29969758 | 41835 | 8: 33203967 |
| 41881 | 8: 34438846 | 41903 | 8: 35034999 | 41929 | 8: 35568258 | 41956 | 8: 36096463 | 41973 | 8: 36469080 |
| 41974 | 8: 36489911 | 42016 | 8: 37579398 | 42038 | 8: 38343034 | 42071 | 8: 39659087 | 42104 | 8: 40219724 |
| 42149 | 8: 41244408 | 42172 | 8: 41710832 | 42173 | 8: 41735284 | 42187 | 8: 42123936 | 42190 | 8: 42204721 |
| 42208 | 8: 42686594 | 42212 | 8: 42720374 | 42216 | 8: 42880518 | 42241 | 8: 43615936 | 42271 | 8: 44484817 |
| 42312 | 8: 45608581 | 42314 | 8: 45672590 | 42329 | 8: 45953160 | 42330 | 8: 45985343 | 42332 | 8: 46013064 |
| 42395 | 8: 47420937 | 42418 | 8: 47998648 | 42466 | 8: 49248066 | 42519 | 8: 50846856 | 42524 | 8: 50995576 |
| 42565 | 8: 52018600 | 42588 | 8: 52629220 | 42591 | 8: 52674907 | 42603 | 8: 52986604 | 42661 | 8: 54554681 |
| 42683 | 8: 55077468 | 42694 | 8: 55493961 | 42765 | 8: 57313074 | 42849 | 8: 59626198 | 42853 | 8: 59747078 |
| 42916 | 8: 61134883 | 42918 | 8: 61212042 | 42919 | 8: 61235958 | 42922 | 8: 61366316 | 42925 | 8: 61452677 |
| 43033 | 8: 64222797 | 43049 | 8: 64876145 | 43067 | 8: 65260788 | 43080 | 8: 65506614 | 43087 | 8: 65773835 |
| 43124 | 8: 66668352 | 43200 | 8: 68399674 | 43235 | 8: 69120902 | 43284 | 8: 70429214 | 43306 | 8: 70893052 |
| 43311 | 8: 70996588 | 43380 | 8: 72930313 | 43454 | 8: 74772105 | 43457 | 8: 74801687 | 43527 | 8: 76729847 |
| 43563 | 8: 77557071 | 43702 | 8: 81553047 | 43722 | 8: 82063613 | 43764 | 8: 82948559 | 43831 | 8: 84602590 |
| 43865 | 8: 85540348 | 43883 | 8: 85967740 | 43942 | 8: 87290730 | 43979 | 8: 88449390 | 43993 | 8: 88666966 |
| 44077 | 8: 90752165 | 44176 | 8: 93011122 | 44205 | 8: 93906534 | 44262 | 8: 95928376 | 44268 | 8: 96045608 |
| 44269 | 8: 96053216 | 44574 | 8: 102774503 | 44648 | 8: 104180690 | 44652 | 8: 104213734 | 44749 | 8: 106594158 |
| 44887 | 8: 110915706 | 44899 | 8: 111410824 | 45035 | 9: 2664478 | 45036 | 9: 2667437 | 45037 | 9: 2675248 |
| 45038 | 9: 2678406 | 45039 | 9: 2679109 | 45040 | 9: 2681750 | 45041 | 9: 2685486 | 45079 | 9: 3897414 |
| 45192 | 9: 7600098 | 45344 | 9: 10670291 | 45354 | 9: 10721590 | 45379 | 9: 10940149 | 45383 | 9: 11053216 |
| 45423 | 9: 12009299 | 45432 | 9: 12198197 | 45496 | 9: 13287682 | 45535 | 9: 14277874 | 45589 | 9: 15430684 |
| 45660 | 9: 16629763 | 45662 | 9: 16687402 | 45664 | 9: 16727064 | 45676 | 9: 17031856 | 45707 | 9: 18068635 |
| 45744 | 9: 19093497 | 45768 | 9: 19999771 | 45832 | 9: 21796887 | 45872 | 9: 22692551 | 45957 | 9: 24735769 |
| 45958 | 9: 24739623 | 45959 | 9: 24743503 | 45960 | 9: 24744612 | 45961 | 9: 24746634 | 45962 | 9: 24747990 |
| 45963 | 9: 24753059 | 45964 | 9: 24757919 | 45965 | 9: 24760506 | 45991 | 9: 25685943 | 46046 | 9: 27095343 |
| 46063 | 9: 27473901 | 46065 | 9: 27477523 | 46066 | 9: 27485623 | 46191 | 9: 29796827 | 46248 | 9: 31114198 |
| 46259 | 9: 31366005 | 46302 | 9: 32301933 | 46386 | 9: 34555386 | 46398 | 9: 34906969 | 46462 | 9: 36491907 |
| 46486 | 9: 37002303 | 46488 | 9: 37019786 | 46516 | 9: 37778578 | 46518 | 9: 37786775 | 46528 | 9: 37990154 |
| 46609 | 9: 39610171 | 46636 | 9: 39977921 | 46841 | 9: 45495778 | 46843 | 9: 45524861 | 46886 | 9: 46918750 |
| 46890 | 9: 47015135 | 46938 | 9: 48713344 | 46957 | 9: 49281683 | 46958 | 9: 49310703 | 46959 | 9: 49332688 |
| 46969 | 9: 49534025 | 46970 | 9: 49560954 | 47019 | 9: 50797460 | 47025 | 9: 51023481 | 47032 | 9: 51178639 |
| 47062 | 9: 52118530 | 47097 | 9: 52875308 | 47136 | 9: 54016591 | 47139 | 9: 54071333 | 47141 | 9: 54121979 |
| 47143 | 9: 54193058 | 47144 | 9: 54241895 | 47147 | 9: 54281836 | 47156 | 9: 54592540 | 47165 | 9: 54750120 |
| 47200 | 9: 55772680 | 47203 | 9: 55920760 | 47212 | 9: 56203305 | 47237 | 9: 56787230 | 47337 | 9: 59589683 |
| 47486 | 9: 63468566 | 47687 | 9: 67996881 | 47691 | 9: 68095521 | 47755 | 9: 69703203 | 47782 | 9: 70360272 |
| 47785 | 9: 70387952 | 47920 | 9: 73701336 | 48263 | 9: 81076190 | 48268 | 9: 81121582 | 48490 | 9: 85784712 |
| 48495 | 9: 85842378 | 48503 | 9: 86006398 | 48511 | 9: 86271158 | 48513 | 9: 86326905 | 48558 | 9: 87361936 |
| 48562 | 9: 87394371 | 48620 | 9: 88414100 | 48662 | 9: 89255756 | 48663 | 9: 89261575 | 48664 | 9: 89266339 |
| 48691 | 9: 89514561 | 48721 | 9: 90252969 | 48732 | 9: 90513278 | 48805 | 9: 92056243 | 48810 | 9: 92215180 |
| 48813 | 9: 92280929 | 48924 | 9: 94493145 | 49009 | 9: 96178394 | 49069 | 9: 96875482 | 49070 | 9: 96875798 |
| 49073 | 9: 96892725 | 49074 | 9: 96894181 | 49096 | 9: 97251687 | 49102 | 9: 97327317 | 49126 | 9: 97749602 |
| 49382 | 9: 102242225 | 49467 | 9: 103771813 | 49725 | 10: 4803921 | 49789 | 10: 5830788 | 49811 | 10: 6389416 |
| 49838 | 10: 6942796 | 49855 | 10: 7506260 | 49870 | 10: 7723009 | 49879 | 10: 7896282 | 49918 | 10: 8581573 |
| 49929 | 10: 8932852 | 49954 | 10: 9647790 | 49959 | 10: 9706130 | 50090 | 10: 12326160 | 50252 | 10: 15438770 |
| 50302 | 10: 16346333 | 50344 | 10: 17006334 | 50394 | 10: 18062302 | 50406 | 10: 18136100 | 50425 | 10: 18596826 |
| 50457 | 10: 19155115 | 50490 | 10: 19862667 | 50499 | 10: 19959026 | 50578 | 10: 21363314 | 50627 | 10: 22355891 |
| 50628 | 10: 22382830 | 50704 | 10: 26294073 | 50713 | 10: 26447113 | 50738 | 10: 26790460 | 50793 | 10: 27960775 |
| 50811 | 10: 28566378 | 50812 | 10: 28588817 | 50859 | 10: 29085444 | 50868 | 10: 29217264 | 50934 | 10: 31208483 |
| 50952 | 10: 31609187 | 50976 | 10: 32324758 | 51059 | 10: 34358150 | 51094 | 10: 34985737 | 51138 | 10: 35989426 |
| 51141 | 10: 36035947 | 51147 | 10: 36261135 | 51164 | 10: 36819725 | 51193 | 10: 37476784 | 51242 | 10: 38651279 |
| 51294 | 10: 40046559 | 51405 | 10: 43227119 | 51426 | 10: 43996215 | 51527 | 10: 46095471 | 51600 | 10: 47638179 |
| 51609 | 10: 47789244 | 51610 | 10: 47842702 | 51626 | 10: 48320530 | 51629 | 10: 48352886 | 51645 | 10: 48589436 |
| 51649 | 10: 48670398 | 51770 | 10: 51376594 | 51773 | 10: 51492328 | 51780 | 10: 51664061 | 51801 | 10: 52103291 |
| 51809 | 10: 52286224 | 51824 | 10: 52716176 | 51839 | 10: 52867771 | 51896 | 10: 54578642 | 51972 | 10: 56658480 |
| 52166 | 10: 62136409 | 52202 | 10: 62942087 | 52272 | 10: 64614710 | 52278 | 10: 64660791 | 52409 | 10: 67916863 |
| 52410 | 10: 67917609 | 52411 | 10: 67918635 | 52412 | 10: 67919519 | 52416 | 10: 68020797 | 52439 | 10: 68405934 |
| 52471 | 10: 69067745 | 52500 | 10: 69834381 | 52520 | 10: 70404549 | 52616 | 10: 73493859 | 52730 | 10: 76467129 |
| 52964 | 10: 81617198 | 53012 | 10: 82478545 | 53038 | 10: 83193828 | 53039 | 10: 83219578 | 53072 | 10: 83953443 |
| 53105 | 10: 84613620 | 53132 | 10: 85160503 | 53157 | 10: 85605717 | 53228 | 10: 87305772 | 53332 | 10: 89626819 |
| 53351 | 10: 89936666 | 53372 | 10: 90371369 | 53373 | 10: 90396997 | 53380 | 10: 90447669 | 53400 | 10: 90743393 |
| 53411 | 10: 90874981 | 53416 | 10: 91003179 | 53424 | 10: 91160509 | 53468 | 10: 92328861 | 53547 | 10: 94201919 |
| 53622 | 10: 95898502 | 53688 | 10: 97342537 | 53729 | 10: 98302579 | 53731 | 10: 98340013 | 53748 | 10: 98561555 |
| 53769 | 10: 99070199 | 53816 | 10: 100101830 | 53837 | 10: 100508994 | 53879 | 10: 101228895 | 53880 | 10: 101250880 |
| 53936 | 10: 102180727 | 53960 | 10: 102751503 | 54049 | 11: 1244479 | 54111 | 11: 1989902 | 54215 | 11: 3345485 |
| 54221 | 11: 3483025 | 54298 | 11: 5089726 | 54369 | 11: 6729148 | 54415 | 11: 7795378 | 54432 | 11: 8194895 |
| 54454 | 11: 9031832 | 54487 | 11: 9455596 | 54526 | 11: 10334522 | 54533 | 11: 10452880 | 54563 | 11: 11221986 |
| 54593 | 11: 11811943 | 54600 | 11: 12001967 | 54611 | 11: 12371073 | 54687 | 11: 13859460 | 54718 | 11: 15010708 |
| 54778 | 11: 16210947 | 54799 | 11: 16834406 | 54848 | 11: 18344213 | 54849 | 11: 18370765 | 54850 | 11: 18396456 |
| 54959 | 11: 21470638 | 55054 | 11: 23592030 | 55056 | 11: 23656903 | 55060 | 11: 23740579 | 55083 | 11: 24318038 |
| 55092 | 11: 24369984 | 55093 | 11: 24377983 | 55094 | 11: 24393545 | 55096 | 11: 24408850 | 55133 | 11: 25341825 |
| 55231 | 11: 27746425 | 55274 | 11: 28736945 | 55276 | 11: 28761952 | 55291 | 11: 29024122 | 55369 | 11: 30763811 |
| 55384 | 11: 31041776 | 55392 | 11: 31275179 | 55413 | 11: 31882807 | 55416 | 11: 31964009 | 55428 | 11: 32373197 |
| 55474 | 11: 33523211 | 55483 | 11: 33718317 | 55559 | 11: 36181120 | 55589 | 11: 37017087 | 55594 | 11: 37101530 |
| 55604 | 11: 37283583 | 55776 | 11: 41848496 | 55903 | 11: 45028224 | 55916 | 11: 45290515 | 55917 | 11: 45313347 |
| 56006 | 11: 47096563 | 56017 | 11: 47326731 | 56096 | 11: 48675652 | 56133 | 11: 49165587 | 56145 | 11: 49409414 |
| 56148 | 11: 49467653 | 56177 | 11: 50202868 | 56186 | 11: 50607517 | 56242 | 11: 52695227 | 56259 | 11: 53270728 |
| 56262 | 11: 53413882 | 56431 | 11: 57988232 | 56468 | 11: 58918358 | 56472 | 11: 58971050 | 56480 | 11: 59181535 |
| 56524 | 11: 60468952 | 56540 | 11: 60494769 | 56542 | 11: 60517676 | 56544 | 11: 60567347 | 56558 | 11: 60775322 |
| 56559 | 11: 60864552 | 56560 | 11: 60906972 | 56594 | 11: 61768776 | 56624 | 11: 62610532 | 56627 | 11: 62639185 |
| 56629 | 11: 62703391 | 56635 | 11: 62811507 | 56646 | 11: 63105834 | 56648 | 11: 63156170 | 56688 | 11: 63881213 |
| 56694 | 11: 63980697 | 56771 | 11: 66096418 | 56774 | 11: 66141532 | 56792 | 11: 66500339 | 56805 | 11: 66814255 |
| 56807 | 11: 66889704 | 56808 | 11: 66971573 | 56813 | 11: 67084838 | 56826 | 11: 67371438 | 56831 | 11: 67445332 |
| 56833 | 11: 67469876 | 56877 | 11: 67856557 | 56883 | 11: 68062927 | 56892 | 11: 68380416 | 56894 | 11: 68423346 |
| 56911 | 11: 68884476 | 56915 | 11: 68962608 | 56951 | 11: 69554436 | 56976 | 11: 70005013 | 56985 | 11: 70216257 |
| 56988 | 11: 70246998 | 56993 | 11: 70300762 | 56996 | 11: 70378153 | 57042 | 11: 70810858 | 57106 | 11: 72079051 |
| 57111 | 11: 72169040 | 57219 | 11: 74557125 | 57222 | 11: 74693404 | 57231 | 11: 75039779 | 57238 | 11: 75245474 |
| 57239 | 11: 75278281 | 57240 | 11: 75301517 | 57244 | 11: 75330473 | 57261 | 11: 75610538 | 57269 | 11: 75826371 |
| 57278 | 11: 76006290 | 57282 | 11: 76108140 | 57286 | 11: 76291884 | 57299 | 11: 76699098 | 57313 | 11: 77009264 |
| 57332 | 11: 77262392 | 57343 | 11: 77494347 | 57355 | 11: 77817486 | 57389 | 11: 78010446 | 57391 | 11: 78051004 |
| 57414 | 11: 78619581 | 57476 | 11: 79716380 | 57485 | 11: 79945955 | 57486 | 11: 80005355 | 57490 | 11: 80160161 |
| 57509 | 11: 80535106 | 57524 | 11: 80835889 | 57534 | 11: 81336077 | 57547 | 11: 81697133 | 57619 | 11: 82922365 |
| 57628 | 11: 83118146 | 57667 | 11: 84097016 | 57691 | 11: 84931500 | 57733 | 11: 85771451 | 57735 | 11: 85810220 |
| 57756 | 11: 86216179 | 57813 | 11: 87691339 | 57824 | 11: 87901219 | 57825 | 11: 87933139 | 57838 | 11: 87991576 |
| 57856 | 11: 88008802 | 57871 | 11: 88092604 | 57875 | 11: 88114495 | 57883 | 11: 88435971 | 57905 | 11: 88999569 |
| 57918 | 11: 89363137 | 58014 | 11: 91483828 | 58015 | 11: 91524490 | 58040 | 11: 92016623 | 58199 | 11: 95837864 |
| 58223 | 11: 96479338 | 58254 | 11: 97291509 | 58262 | 11: 97559369 | 58265 | 11: 97669268 | 58346 | 11: 99292200 |
| 58369 | 11: 99693004 | 58374 | 11: 99779968 | 58381 | 11: 99910936 | 58425 | 11: 100730926 | 58480 | 11: 101720696 |
| 58706 | 11: 105729430 | 58710 | 11: 105964095 | 58711 | 11: 106026712 | 58887 | 12: 3816364 | 58893 | 12: 4017888 |
| 58924 | 12: 4735467 | 59120 | 12: 10585260 | 59121 | 12: 10614578 | 59249 | 12: 13606719 | 59254 | 12: 13766685 |
| 59263 | 12: 13857247 | 59320 | 12: 15004714 | 59351 | 12: 15565363 | 59376 | 12: 15986894 | 59402 | 12: 16562247 |
| 59446 | 12: 17721084 | 59635 | 12: 22358320 | 59648 | 12: 22684761 | 59650 | 12: 22780186 | 59656 | 12: 22878113 |
| 59657 | 12: 22955187 | 59658 | 12: 22957971 | 59662 | 12: 23024428 | 59686 | 12: 23553969 | 59699 | 12: 23968918 |
| 59725 | 12: 24562389 | 59751 | 12: 25043912 | 59818 | 12: 26941585 | 59863 | 12: 28021962 | 59864 | 12: 28031415 |
| 59901 | 12: 29149304 | 59931 | 12: 30073919 | 59940 | 12: 30237550 | 59970 | 12: 30965466 | 59975 | 12: 30995990 |
| 60105 | 12: 34339978 | 60136 | 12: 35202030 | 60146 | 12: 35329106 | 60185 | 12: 36506500 | 60202 | 12: 37059151 |
| 60204 | 12: 37117228 | 60206 | 12: 37121439 | 60213 | 12: 37336023 | 60264 | 12: 39602051 | 60268 | 12: 39726857 |
| 60368 | 12: 43543060 | 60451 | 12: 46609222 | 60452 | 12: 46632594 | 60607 | 12: 50328249 | 60608 | 12: 50367458 |
| 60610 | 12: 50405882 | 60655 | 12: 51430846 | 60683 | 12: 52226211 | 60688 | 12: 52270911 | 60735 | 12: 53308347 |
| 60768 | 12: 53915464 | 60769 | 12: 53924609 | 60780 | 12: 54114793 | 60908 | 12: 57839576 | 60967 | 12: 59688270 |
| 61136 | 12: 65124144 | 61209 | 12: 67262559 | 61285 | 12: 68831445 | 61316 | 12: 69427741 | 61337 | 12: 69681738 |
| 61357 | 12: 70744278 | 61358 | 12: 70747193 | 61411 | 12: 72948314 | 61418 | 12: 73037670 | 61419 | 12: 73063509 |
| 61433 | 12: 73378861 | 61437 | 12: 73452468 | 61748 | 12: 80191366 | 61809 | 12: 81602505 | 61930 | 12: 84189393 |
| 62050 | 12: 86604613 | 62158 | 13: 1818757 | 62173 | 13: 2108292 | 62212 | 13: 2694888 | 62217 | 13: 2721397 |
| 62219 | 13: 2775064 | 62220 | 13: 2800282 | 62221 | 13: 2813363 | 62257 | 13: 3832665 | 62281 | 13: 4414592 |
| 62310 | 13: 5228105 | 62405 | 13: 7418765 | 62519 | 13: 10200560 | 62521 | 13: 10297467 | 62539 | 13: 10605573 |
| 62667 | 13: 14736984 | 62723 | 13: 16382635 | 62724 | 13: 16385352 | 62728 | 13: 16481332 | 62729 | 13: 16481881 |
| 62730 | 13: 16482962 | 62731 | 13: 16484064 | 62732 | 13: 16486013 | 62733 | 13: 16487474 | 62734 | 13: 16489553 |
| 62735 | 13: 16494712 | 62736 | 13: 16495172 | 62737 | 13: 16495924 | 62738 | 13: 16496828 | 62739 | 13: 16497723 |
| 62740 | 13: 16498500 | 62741 | 13: 16499450 | 62742 | 13: 16500106 | 62743 | 13: 16505307 | 62774 | 13: 17297996 |
| 62785 | 13: 17376202 | 62832 | 13: 18167455 | 62850 | 13: 18708210 | 62851 | 13: 18716183 | 62853 | 13: 18721208 |
| 62854 | 13: 18721251 | 62855 | 13: 18723232 | 62856 | 13: 18723968 | 62857 | 13: 18724391 | 62858 | 13: 18724682 |
| 62859 | 13: 18724926 | 62860 | 13: 18725319 | 62861 | 13: 18725595 | 62862 | 13: 18725670 | 62863 | 13: 18725747 |
| 62864 | 13: 18726353 | 62865 | 13: 18727332 | 62866 | 13: 18727382 | 62867 | 13: 18728046 | 62868 | 13: 18728216 |
| 62869 | 13: 18729395 | 62870 | 13: 18729428 | 62871 | 13: 18729514 | 62872 | 13: 18729597 | 62873 | 13: 18732582 |
| 62874 | 13: 18733271 | 62875 | 13: 18733719 | 62876 | 13: 18733742 | 62877 | 13: 18733760 | 62878 | 13: 18733884 |
| 62879 | 13: 18735443 | 62882 | 13: 18791943 | 62931 | 13: 19690250 | 62932 | 13: 19710676 | 62940 | 13: 19814205 |
| 62956 | 13: 20094078 | 62957 | 13: 20180098 | 62960 | 13: 20267627 | 62965 | 13: 20376037 | 62975 | 13: 20455892 |
| 62984 | 13: 20631768 | 63067 | 13: 22364650 | 63079 | 13: 22622712 | 63125 | 13: 24378395 | 63206 | 13: 26030778 |
| 63286 | 13: 28051297 | 63307 | 13: 28631602 | 63318 | 13: 28789636 | 63452 | 13: 31297457 | 63463 | 13: 31535167 |
| 63484 | 13: 32188070 | 63485 | 13: 32192701 | 63486 | 13: 32195543 | 63488 | 13: 32202239 | 63489 | 13: 32206561 |
| 63490 | 13: 32214896 | 63498 | 13: 32363871 | 63515 | 13: 32606184 | 63519 | 13: 32649822 | 63534 | 13: 32908367 |
| 63643 | 13: 35552308 | 63655 | 13: 35893139 | 63853 | 13: 40115588 | 63937 | 13: 42022321 | 63954 | 13: 42455044 |
| 63957 | 13: 42527459 | 63962 | 13: 42563978 | 63996 | 13: 42702842 | 64016 | 13: 43224017 | 64031 | 13: 43618688 |
| 64043 | 13: 43868655 | 64084 | 13: 44564660 | 64090 | 13: 44735441 | 64093 | 13: 44846012 | 64094 | 13: 44877072 |
| 64095 | 13: 44877728 | 64096 | 13: 44883203 | 64097 | 13: 44884376 | 64098 | 13: 44891644 | 64099 | 13: 44900803 |
| 64100 | 13: 44901430 | 64101 | 13: 44902570 | 64102 | 13: 44908683 | 64104 | 13: 44929091 | 64106 | 13: 44941064 |
| 64107 | 13: 44945347 | 64108 | 13: 44954742 | 64109 | 13: 44957480 | 64110 | 13: 44959530 | 64111 | 13: 44971418 |
| 64113 | 13: 44974314 | 64114 | 13: 44977218 | 64118 | 13: 44984974 | 64120 | 13: 44991424 | 64123 | 13: 44994329 |
| 64124 | 13: 44996032 | 64161 | 13: 45580238 | 64220 | 13: 46808920 | 64238 | 13: 47284141 | 64266 | 13: 47867949 |
| 64336 | 13: 49568372 | 64337 | 13: 49569350 | 64338 | 13: 49570779 | 64339 | 13: 49571658 | 64416 | 13: 52005450 |
| 64426 | 13: 52366895 | 64431 | 13: 52518024 | 64456 | 13: 52984857 | 64458 | 13: 53006663 | 64466 | 13: 53163449 |
| 64512 | 13: 54065681 | 64629 | 13: 57014498 | 64667 | 13: 57988686 | 64672 | 13: 58092691 | 64887 | 13: 62737371 |
| 64900 | 13: 62849693 | 64901 | 13: 62871349 | 64910 | 13: 63113668 | 64979 | 13: 64765574 | 64992 | 13: 65197131 |
| 65003 | 13: 65421742 | 65046 | 13: 66169238 | 65096 | 13: 67321578 | 65143 | 13: 68639312 | 65145 | 13: 68664064 |
| 65159 | 13: 69019407 | 65312 | 13: 72652771 | 65332 | 13: 73024654 | 65430 | 13: 74713520 | 65439 | 13: 75021624 |
| 65447 | 13: 75318964 | 65448 | 13: 75354084 | 65503 | 13: 76376258 | 65507 | 13: 76438492 | 65517 | 13: 76641970 |
| 65529 | 13: 76798256 | 65764 | 13: 81126899 | 65905 | 14: 1006940 | 65963 | 14: 2212373 | 65993 | 14: 3074708 |
| 65995 | 14: 3098041 | 66091 | 14: 4323988 | 66140 | 14: 5386856 | 66177 | 14: 5966351 | 66207 | 14: 6303201 |
| 66289 | 14: 7818027 | 66305 | 14: 8003665 | 66326 | 14: 8349092 | 66332 | 14: 8441986 | 66350 | 14: 8630299 |
| 66373 | 14: 8996524 | 66493 | 14: 11272085 | 66494 | 14: 11275856 | 66495 | 14: 11277881 | 66496 | 14: 11278042 |
| 66497 | 14: 11281126 | 66499 | 14: 11294774 | 66500 | 14: 11299050 | 66547 | 14: 12222307 | 66586 | 14: 12811226 |
| 66627 | 14: 14043986 | 66687 | 14: 15518676 | 66690 | 14: 15530827 | 66691 | 14: 15559743 | 66692 | 14: 15601008 |
| 66693 | 14: 15625272 | 66694 | 14: 15626105 | 66695 | 14: 15631968 | 66696 | 14: 15643325 | 66698 | 14: 15672366 |
| 66717 | 14: 16009930 | 66718 | 14: 16010978 | 66719 | 14: 16022809 | 66724 | 14: 16161546 | 66750 | 14: 16670887 |
| 66775 | 14: 17233811 | 66795 | 14: 17760515 | 66799 | 14: 17770830 | 66831 | 14: 18480356 | 66832 | 14: 18510376 |
| 66909 | 14: 20574088 | 66969 | 14: 21764414 | 66976 | 14: 21914329 | 66987 | 14: 22199112 | 66989 | 14: 22291692 |
| 66991 | 14: 22353534 | 66994 | 14: 22402037 | 66997 | 14: 22459009 | 66998 | 14: 22460066 | 66999 | 14: 22492158 |
| 67000 | 14: 22499302 | 67001 | 14: 22499882 | 67002 | 14: 22500823 | 67003 | 14: 22500981 | 67004 | 14: 22501165 |
| 67005 | 14: 22502609 | 67006 | 14: 22503786 | 67007 | 14: 22505668 | 67009 | 14: 22551848 | 67010 | 14: 22559672 |
| 67011 | 14: 22559976 | 67012 | 14: 22560498 | 67013 | 14: 22560939 | 67014 | 14: 22561597 | 67015 | 14: 22562389 |
| 67016 | 14: 22562808 | 67017 | 14: 22562827 | 67018 | 14: 22563098 | 67019 | 14: 22563125 | 67020 | 14: 22563136 |
| 67021 | 14: 22563534 | 67022 | 14: 22564168 | 67023 | 14: 22564379 | 67024 | 14: 22564874 | 67025 | 14: 22565102 |
| 67026 | 14: 22565724 | 67027 | 14: 22565752 | 67028 | 14: 22565896 | 67029 | 14: 22565903 | 67030 | 14: 22566273 |
| 67031 | 14: 22566433 | 67032 | 14: 22566621 | 67033 | 14: 22567218 | 67034 | 14: 22567252 | 67035 | 14: 22567396 |
| 67036 | 14: 22567418 | 67037 | 14: 22567497 | 67038 | 14: 22567570 | 67039 | 14: 22568006 | 67040 | 14: 22568446 |
| 67041 | 14: 22568460 | 67042 | 14: 22568517 | 67043 | 14: 22568557 | 67044 | 14: 22568700 | 67045 | 14: 22568919 |
| 67046 | 14: 22569314 | 67047 | 14: 22569464 | 67048 | 14: 22569577 | 67049 | 14: 22569581 | 67050 | 14: 22569980 |
| 67051 | 14: 22570044 | 67052 | 14: 22570058 | 67053 | 14: 22570080 | 67054 | 14: 22570301 | 67055 | 14: 22570448 |
| 67056 | 14: 22570466 | 67057 | 14: 22570726 | 67058 | 14: 22571422 | 67059 | 14: 22573154 | 67060 | 14: 22574799 |
| 67061 | 14: 22579399 | 67062 | 14: 22579809 | 67063 | 14: 22581401 | 67064 | 14: 22584062 | 67065 | 14: 22584452 |
| 67066 | 14: 22592312 | 67071 | 14: 22671158 | 67072 | 14: 22680795 | 67073 | 14: 22681826 | 67079 | 14: 22781305 |
| 67080 | 14: 22830132 | 67081 | 14: 22839488 | 67082 | 14: 22841040 | 67083 | 14: 22842180 | 67084 | 14: 22861656 |
| 67085 | 14: 22861757 | 67086 | 14: 22861840 | 67087 | 14: 22866278 | 67088 | 14: 22867321 | 67089 | 14: 22870443 |
| 67090 | 14: 22872231 | 67091 | 14: 22872707 | 67092 | 14: 22875389 | 67094 | 14: 22883080 | 67096 | 14: 22887406 |
| 67097 | 14: 22888105 | 67098 | 14: 22895284 | 67099 | 14: 22896212 | 67100 | 14: 22899088 | 67101 | 14: 22900130 |
| 67102 | 14: 22900140 | 67103 | 14: 22903945 | 67104 | 14: 22904357 | 67105 | 14: 22905670 | 67106 | 14: 22915081 |
| 67107 | 14: 22915304 | 67108 | 14: 22916046 | 67109 | 14: 22916365 | 67110 | 14: 22917130 | 67111 | 14: 22917282 |
| 67112 | 14: 22918679 | 67113 | 14: 22919099 | 67114 | 14: 22922271 | 67115 | 14: 22925305 | 67116 | 14: 22926221 |
| 67117 | 14: 22933624 | 67118 | 14: 22935277 | 67119 | 14: 22940329 | 67120 | 14: 22941327 | 67121 | 14: 22944551 |
| 67122 | 14: 22945851 | 67123 | 14: 22950339 | 67124 | 14: 22951594 | 67125 | 14: 22952187 | 67126 | 14: 22952264 |
| 67127 | 14: 22958318 | 67128 | 14: 22959278 | 67129 | 14: 22962984 | 67130 | 14: 22963530 | 67131 | 14: 22963552 |
| 67132 | 14: 22963956 | 67133 | 14: 22964530 | 67134 | 14: 22968925 | 67135 | 14: 22969225 | 67136 | 14: 22969659 |
| 67137 | 14: 22970008 | 67138 | 14: 22983250 | 67139 | 14: 22983836 | 67140 | 14: 22986080 | 67141 | 14: 22989108 |
| 67142 | 14: 22989880 | 67143 | 14: 22989917 | 67144 | 14: 22995750 | 67145 | 14: 22995861 | 67146 | 14: 22999212 |
| 67147 | 14: 22999475 | 67148 | 14: 23000859 | 67149 | 14: 23001109 | 67150 | 14: 23001803 | 67151 | 14: 23004072 |
| 67152 | 14: 23005237 | 67153 | 14: 23009266 | 67157 | 14: 23168386 | 67175 | 14: 23216693 | 67180 | 14: 23217714 |
| 67181 | 14: 23217718 | 67183 | 14: 23219140 | 67189 | 14: 23221714 | 67191 | 14: 23232498 | 67193 | 14: 23241336 |
| 67194 | 14: 23244133 | 67195 | 14: 23245996 | 67196 | 14: 23246004 | 67197 | 14: 23246030 | 67198 | 14: 23246116 |
| 67199 | 14: 23246121 | 67200 | 14: 23246668 | 67201 | 14: 23247018 | 67202 | 14: 23247588 | 67203 | 14: 23247661 |
| 67204 | 14: 23247870 | 67205 | 14: 23247878 | 67206 | 14: 23247920 | 67207 | 14: 23247940 | 67208 | 14: 23248135 |
| 67209 | 14: 23248201 | 67210 | 14: 23248211 | 67211 | 14: 23248448 | 67212 | 14: 23248692 | 67213 | 14: 23248770 |
| 67214 | 14: 23248888 | 67215 | 14: 23248943 | 67216 | 14: 23249702 | 67217 | 14: 23249738 | 67218 | 14: 23249950 |
| 67219 | 14: 23249976 | 67220 | 14: 23250082 | 67221 | 14: 23250315 | 67222 | 14: 23250686 | 67223 | 14: 23250690 |
| 67224 | 14: 23250828 | 67225 | 14: 23250871 | 67226 | 14: 23250914 | 67227 | 14: 23251020 | 67228 | 14: 23251302 |
| 67229 | 14: 23251581 | 67230 | 14: 23251684 | 67231 | 14: 23252354 | 67232 | 14: 23252429 | 67233 | 14: 23252563 |
| 67234 | 14: 23252757 | 67235 | 14: 23252809 | 67236 | 14: 23253043 | 67237 | 14: 23253210 | 67238 | 14: 23253295 |
| 67239 | 14: 23253300 | 67240 | 14: 23253568 | 67241 | 14: 23253746 | 67242 | 14: 23253786 | 67243 | 14: 23253805 |
| 67244 | 14: 23254426 | 67245 | 14: 23254453 | 67246 | 14: 23254563 | 67247 | 14: 23254639 | 67248 | 14: 23254769 |
| 67249 | 14: 23254807 | 67250 | 14: 23254919 | 67251 | 14: 23255007 | 67252 | 14: 23255159 | 67253 | 14: 23255587 |
| 67254 | 14: 23255824 | 67255 | 14: 23256710 | 67256 | 14: 23257050 | 67257 | 14: 23257092 | 67258 | 14: 23257183 |
| 67259 | 14: 23257229 | 67260 | 14: 23257379 | 67261 | 14: 23257441 | 67262 | 14: 23257644 | 67263 | 14: 23257665 |
| 67264 | 14: 23257775 | 67265 | 14: 23258452 | 67266 | 14: 23258542 | 67267 | 14: 23262536 | 67268 | 14: 23262544 |
| 67269 | 14: 23262984 | 67270 | 14: 23264575 | 67271 | 14: 23264736 | 67272 | 14: 23265786 | 67273 | 14: 23265991 |
| 67274 | 14: 23266384 | 67275 | 14: 23271813 | 67276 | 14: 23273276 | 67277 | 14: 23273801 | 67278 | 14: 23274957 |
| 67279 | 14: 23277049 | 67280 | 14: 23278232 | 67281 | 14: 23278652 | 67282 | 14: 23278927 | 67283 | 14: 23279593 |
| 67284 | 14: 23279712 | 67285 | 14: 23285130 | 67286 | 14: 23288477 | 67287 | 14: 23288921 | 67288 | 14: 23289158 |
| 67295 | 14: 23298062 | 67296 | 14: 23300304 | 67297 | 14: 23314460 | 67298 | 14: 23314730 | 67299 | 14: 23314761 |
| 67300 | 14: 23321577 | 67301 | 14: 23326588 | 67302 | 14: 23338890 | 67303 | 14: 23343150 | 67304 | 14: 23346065 |
| 67306 | 14: 23383008 | 67308 | 14: 23392938 | 67309 | 14: 23408928 | 67310 | 14: 23409204 | 67311 | 14: 23409494 |
| 67312 | 14: 23410644 | 67313 | 14: 23410867 | 67314 | 14: 23411712 | 67315 | 14: 23414348 | 67316 | 14: 23414443 |
| 67317 | 14: 23421256 | 67318 | 14: 23421807 | 67319 | 14: 23422909 | 67320 | 14: 23423768 | 67321 | 14: 23423823 |
| 67322 | 14: 23424726 | 67323 | 14: 23426056 | 67324 | 14: 23427548 | 67325 | 14: 23429529 | 67326 | 14: 23430628 |
| 67327 | 14: 23430999 | 67328 | 14: 23431167 | 67329 | 14: 23438214 | 67330 | 14: 23439941 | 67331 | 14: 23440995 |
| 67332 | 14: 23441483 | 67333 | 14: 23443135 | 67334 | 14: 23444334 | 67335 | 14: 23446328 | 67336 | 14: 23447775 |
| 67337 | 14: 23450091 | 67338 | 14: 23456836 | 67339 | 14: 23458201 | 67340 | 14: 23459603 | 67343 | 14: 23528515 |
| 67344 | 14: 23532117 | 67354 | 14: 23600267 | 67355 | 14: 23607944 | 67356 | 14: 23622754 | 67357 | 14: 23630896 |
| 67358 | 14: 23639458 | 67359 | 14: 23652804 | 67368 | 14: 23825669 | 67370 | 14: 23829057 | 67371 | 14: 23829066 |
| 67372 | 14: 23829235 | 67373 | 14: 23830314 | 67375 | 14: 23831223 | 67376 | 14: 23831351 | 67377 | 14: 23831451 |
| 67378 | 14: 23831720 | 67379 | 14: 23832062 | 67380 | 14: 23832448 | 67381 | 14: 23832632 | 67382 | 14: 23832846 |
| 67383 | 14: 23832941 | 67384 | 14: 23833173 | 67385 | 14: 23833835 | 67386 | 14: 23834262 | 67387 | 14: 23834735 |
| 67388 | 14: 23834925 | 67389 | 14: 23835136 | 67390 | 14: 23835627 | 67391 | 14: 23835870 | 67392 | 14: 23835971 |
| 67393 | 14: 23836131 | 67394 | 14: 23836398 | 67395 | 14: 23836443 | 67396 | 14: 23836603 | 67397 | 14: 23836691 |
| 67398 | 14: 23836914 | 67399 | 14: 23837002 | 67400 | 14: 23838557 | 67401 | 14: 23839177 | 67402 | 14: 23840827 |
| 67403 | 14: 23842863 | 67404 | 14: 23848780 | 67405 | 14: 23848797 | 67406 | 14: 23848827 | 67407 | 14: 23849503 |
| 67408 | 14: 23852492 | 67409 | 14: 23852815 | 67410 | 14: 23852921 | 67411 | 14: 23853184 | 67412 | 14: 23853223 |
| 67413 | 14: 23854487 | 67414 | 14: 23856672 | 67415 | 14: 23860303 | 67416 | 14: 23862417 | 67418 | 14: 23868149 |
| 67419 | 14: 23882209 | 67420 | 14: 23887365 | 67421 | 14: 23887370 | 67422 | 14: 23937950 | 67423 | 14: 23945118 |
| 67425 | 14: 23992830 | 67426 | 14: 24019648 | 67430 | 14: 24107226 | 67436 | 14: 24297582 | 67440 | 14: 24350585 |
| 67446 | 14: 24477168 | 67447 | 14: 24512384 | 67449 | 14: 24516122 | 67450 | 14: 24519148 | 67451 | 14: 24519442 |
| 67452 | 14: 24520182 | 67453 | 14: 24522732 | 67454 | 14: 24524138 | 67455 | 14: 24527219 | 67456 | 14: 24528754 |
| 67457 | 14: 24532678 | 67458 | 14: 24535392 | 67459 | 14: 24536887 | 67460 | 14: 24537213 | 67461 | 14: 24537249 |
| 67462 | 14: 24537652 | 67463 | 14: 24537937 | 67464 | 14: 24538192 | 67465 | 14: 24538201 | 67466 | 14: 24538525 |
| 67467 | 14: 24538984 | 67468 | 14: 24539180 | 67469 | 14: 24539228 | 67470 | 14: 24539315 | 67471 | 14: 24539324 |
| 67472 | 14: 24539556 | 67473 | 14: 24539584 | 67474 | 14: 24539696 | 67475 | 14: 24540007 | 67476 | 14: 24540057 |
| 67477 | 14: 24540244 | 67478 | 14: 24540270 | 67479 | 14: 24542751 | 67481 | 14: 24571024 | 67482 | 14: 24580144 |
| 67483 | 14: 24587368 | 67484 | 14: 24594780 | 67485 | 14: 24604764 | 67486 | 14: 24609404 | 67487 | 14: 24618912 |
| 67488 | 14: 24644457 | 67493 | 14: 24767026 | 67495 | 14: 24790463 | 67496 | 14: 24825146 | 67497 | 14: 24859655 |
| 67500 | 14: 24940746 | 67501 | 14: 24941221 | 67502 | 14: 24967239 | 67503 | 14: 24967988 | 67504 | 14: 24973247 |
| 67505 | 14: 24978104 | 67506 | 14: 24979482 | 67507 | 14: 24982302 | 67508 | 14: 24983741 | 67509 | 14: 24986291 |
| 67510 | 14: 25002052 | 67511 | 14: 25030583 | 67513 | 14: 25082860 | 67516 | 14: 25152867 | 67517 | 14: 25158355 |
| 67526 | 14: 25266063 | 67530 | 14: 25440652 | 67533 | 14: 25548588 | 67534 | 14: 25548611 | 67535 | 14: 25548858 |
| 67536 | 14: 25549944 | 67537 | 14: 25550026 | 67538 | 14: 25550835 | 67539 | 14: 25550879 | 67540 | 14: 25551206 |
| 67541 | 14: 25551423 | 67542 | 14: 25551661 | 67543 | 14: 25551868 | 67544 | 14: 25552256 | 67545 | 14: 25552486 |
| 67546 | 14: 25552782 | 67547 | 14: 25552984 | 67548 | 14: 25553034 | 67549 | 14: 25553066 | 67550 | 14: 25553217 |
| 67552 | 14: 25555831 | 67613 | 14: 26283998 | 67621 | 14: 26472341 | 67627 | 14: 26644064 | 67628 | 14: 26666519 |
| 67686 | 14: 28503857 | 67690 | 14: 28611968 | 67696 | 14: 28706164 | 67714 | 14: 29246244 | 67717 | 14: 29321450 |
| 67723 | 14: 29455092 | 67743 | 14: 29818291 | 67756 | 14: 30089537 | 67838 | 14: 32001174 | 67864 | 14: 32341495 |
| 67882 | 14: 32807225 | 67883 | 14: 32884654 | 67887 | 14: 32989933 | 67910 | 14: 33408077 | 67918 | 14: 33505061 |
| 67920 | 14: 33551756 | 67973 | 14: 34950627 | 67979 | 14: 35239973 | 67980 | 14: 35240016 | 67981 | 14: 35240021 |
| 67982 | 14: 35240029 | 67983 | 14: 35240427 | 67984 | 14: 35240523 | 67985 | 14: 35240587 | 67986 | 14: 35240759 |
| 67987 | 14: 35240947 | 67988 | 14: 35241115 | 67989 | 14: 35241328 | 67990 | 14: 35241474 | 67991 | 14: 35241672 |
| 67992 | 14: 35265340 | 67998 | 14: 35425257 | 68003 | 14: 35595531 | 68006 | 14: 35751572 | 68010 | 14: 35894072 |
| 68024 | 14: 36171691 | 68025 | 14: 36225604 | 68027 | 14: 36267667 | 68045 | 14: 36581802 | 68070 | 14: 37486005 |
| 68085 | 14: 37852302 | 68092 | 14: 38020858 | 68098 | 14: 38241778 | 68104 | 14: 38511542 | 68107 | 14: 38549650 |
| 68121 | 14: 38865578 | 68136 | 14: 39381019 | 68138 | 14: 39446327 | 68154 | 14: 39861013 | 68181 | 14: 40565849 |
| 68184 | 14: 40702265 | 68188 | 14: 40809782 | 68195 | 14: 41149021 | 68202 | 14: 41340996 | 68219 | 14: 41739836 |
| 68239 | 14: 42191184 | 68242 | 14: 42286395 | 68265 | 14: 42846993 | 68302 | 14: 44079419 | 68338 | 14: 44933472 |
| 68350 | 14: 45112392 | 68358 | 14: 45283009 | 68368 | 14: 45610432 | 68374 | 14: 45681264 | 68376 | 14: 45772293 |
| 68391 | 14: 46030748 | 68402 | 14: 46236143 | 68403 | 14: 46236637 | 68404 | 14: 46237110 | 68405 | 14: 46238761 |
| 68406 | 14: 46241868 | 68407 | 14: 46244088 | 68408 | 14: 46250918 | 68418 | 14: 46418593 | 68424 | 14: 46603107 |
| 68441 | 14: 46995812 | 68444 | 14: 47059409 | 68474 | 14: 48033192 | 68483 | 14: 48202902 | 68512 | 14: 48835114 |
| 68819 | 14: 56925358 | 68973 | 14: 60573159 | 68974 | 14: 60591022 | 69042 | 14: 62002265 | 69055 | 14: 62159723 |
| 69056 | 14: 62160097 | 69093 | 14: 62960532 | 69145 | 14: 64219535 | 69158 | 14: 64538337 | 69236 | 14: 66565905 |
| 69296 | 14: 67751963 | 69405 | 14: 70557126 | 69479 | 14: 72621724 | 69527 | 14: 73767156 | 69546 | 14: 74251050 |
| 69666 | 14: 77010786 | 69676 | 14: 77348388 | 69701 | 14: 77989698 | 69824 | 14: 80695704 | 69841 | 14: 81015684 |
| 70102 | 15: 4692832 | 70287 | 15: 5550484 | 70295 | 15: 5724091 | 70403 | 15: 8239471 | 70559 | 15: 13400749 |
| 70578 | 15: 14002939 | 70584 | 15: 14158577 | 70619 | 15: 14996109 | 70634 | 15: 15286305 | 70711 | 15: 16549253 |
| 70719 | 15: 16763241 | 70746 | 15: 17193061 | 70781 | 15: 18160360 | 70783 | 15: 18238844 | 70920 | 15: 21418237 |
| 70933 | 15: 21792771 | 70955 | 15: 22295223 | 71033 | 15: 23794449 | 71075 | 15: 24300165 | 71076 | 15: 24314377 |
| 71084 | 15: 24406959 | 71092 | 15: 24625723 | 71106 | 15: 24813538 | 71225 | 15: 27497541 | 71246 | 15: 28011102 |
| 71266 | 15: 28279946 | 71274 | 15: 28453282 | 71275 | 15: 28497953 | 71313 | 15: 28951920 | 71373 | 15: 30506918 |
| 71421 | 15: 31626657 | 71443 | 15: 32064162 | 71459 | 15: 32448502 | 71467 | 15: 32684505 | 71489 | 15: 33066384 |
| 71494 | 15: 33140066 | 71500 | 15: 33283643 | 71516 | 15: 33757310 | 71519 | 15: 33784264 | 71542 | 15: 34144843 |
| 71544 | 15: 34205212 | 71548 | 15: 34249244 | 71550 | 15: 34273013 | 71565 | 15: 34545054 | 71594 | 15: 35124174 |
| 71637 | 15: 36171696 | 71643 | 15: 36281231 | 71644 | 15: 36316689 | 71668 | 15: 36835278 | 71670 | 15: 36913421 |
| 71721 | 15: 38013431 | 71735 | 15: 38345972 | 71814 | 15: 40225597 | 71892 | 15: 41883212 | 71918 | 15: 42285508 |
| 71919 | 15: 42310362 | 71922 | 15: 42461633 | 71961 | 15: 43061218 | 71966 | 15: 43113772 | 71983 | 15: 43322372 |
| 72013 | 15: 43808690 | 72034 | 15: 44213989 | 72038 | 15: 44321644 | 72039 | 15: 44348210 | 72041 | 15: 44377304 |
| 72043 | 15: 44404296 | 72081 | 15: 45199864 | 72107 | 15: 45767348 | 72150 | 15: 46515608 | 72163 | 15: 46717734 |
| 72166 | 15: 46782723 | 72172 | 15: 46863275 | 72181 | 15: 47142381 | 72226 | 15: 48040954 | 72245 | 15: 48275779 |
| 72278 | 15: 49421011 | 72280 | 15: 49469161 | 72294 | 15: 49994423 | 72324 | 15: 51010384 | 72368 | 15: 51564179 |
| 72387 | 15: 51844292 | 72392 | 15: 51981969 | 72395 | 15: 51992306 | 72396 | 15: 52000306 | 72403 | 15: 52160095 |
| 72434 | 15: 52969357 | 72473 | 15: 53341502 | 72559 | 15: 55149996 | 72592 | 15: 56124060 | 72635 | 15: 56767431 |
| 72636 | 15: 56774921 | 72637 | 15: 56780183 | 72638 | 15: 56782530 | 72639 | 15: 56783614 | 72640 | 15: 56784736 |
| 72641 | 15: 56785971 | 72642 | 15: 56797332 | 72643 | 15: 56807110 | 72646 | 15: 56815479 | 72647 | 15: 56818346 |
| 72648 | 15: 56829119 | 72649 | 15: 56831561 | 72650 | 15: 56832894 | 72651 | 15: 56833644 | 72652 | 15: 56834347 |
| 72653 | 15: 56835434 | 72654 | 15: 56836055 | 72655 | 15: 56836949 | 72656 | 15: 56838230 | 72657 | 15: 56838871 |
| 72658 | 15: 56840204 | 72659 | 15: 56841525 | 72660 | 15: 56842815 | 72661 | 15: 56843668 | 72662 | 15: 56845155 |
| 72663 | 15: 56851874 | 72664 | 15: 56853748 | 72665 | 15: 56855128 | 72666 | 15: 56856657 | 72667 | 15: 56868653 |
| 72668 | 15: 56869977 | 72690 | 15: 57484879 | 72695 | 15: 57536250 | 72767 | 15: 58882110 | 72769 | 15: 58979088 |
| 72784 | 15: 59472006 | 72833 | 15: 60847989 | 72869 | 15: 61639615 | 72884 | 15: 61810673 | 72900 | 15: 62235431 |
| 72906 | 15: 62456943 | 72909 | 15: 62484563 | 72910 | 15: 62511268 | 72918 | 15: 62618258 | 72922 | 15: 62694367 |
| 72976 | 15: 63833091 | 72988 | 15: 64092090 | 73022 | 15: 64696473 | 73048 | 15: 65009502 | 73052 | 15: 65067229 |
| 73130 | 15: 66545124 | 73132 | 15: 66614036 | 73460 | 15: 75001650 | 73465 | 15: 75074384 | 73560 | 15: 77284842 |
| 73564 | 15: 77342823 | 73599 | 15: 77992295 | 73602 | 15: 78001169 | 73610 | 15: 78017138 | 73626 | 15: 78135787 |
| 73719 | 15: 80696386 | 73731 | 15: 81021484 | 73760 | 15: 81377169 | 73768 | 15: 81639702 | 73794 | 15: 82390046 |
| 73813 | 15: 82751747 | 73814 | 15: 82766106 | 73826 | 15: 82951372 | 73834 | 15: 83031817 | 73835 | 15: 83043883 |
| 73836 | 15: 83046338 | 73837 | 15: 83054032 | 73838 | 15: 83058015 | 73839 | 15: 83063624 | 73945 | 16: 1306469 |
| 73949 | 16: 1350556 | 73955 | 16: 1432322 | 74009 | 16: 2521425 | 74012 | 16: 2584565 | 74027 | 16: 3043683 |
| 74058 | 16: 3453206 | 74076 | 16: 3954831 | 74079 | 16: 3986796 | 74218 | 16: 7674017 | 74248 | 16: 8845857 |
| 74278 | 16: 9938750 | 74286 | 16: 10168137 | 74356 | 16: 12347316 | 74366 | 16: 12489625 | 74382 | 16: 12870890 |
| 74389 | 16: 12974308 | 74390 | 16: 12974917 | 74391 | 16: 12975122 | 74392 | 16: 12975220 | 74393 | 16: 12975427 |
| 74394 | 16: 12975489 | 74395 | 16: 12975573 | 74396 | 16: 12975644 | 74460 | 16: 14726498 | 74462 | 16: 14841844 |
| 74520 | 16: 16433701 | 74547 | 16: 17397768 | 74579 | 16: 18551432 | 74583 | 16: 18633047 | 74592 | 16: 18835344 |
| 4621 | 16: 19568001 | 74641 | 16: 20043212 | 74669 | 16: 20811063 | 74702 | 16: 21584184 | 74853 | 16: 25049887 |
| 74963 | 16: 26927766 | 74986 | 16: 27682201 | 75041 | 16: 29174251 | 75060 | 16: 29760720 | 75062 | 16: 29811081 |
| 75073 | 16: 30136403 | 75075 | 16: 30161226 | 75076 | 16: 30185523 | 75134 | 16: 31704359 | 75143 | 16: 31896105 |
| 75150 | 16: 32035645 | 75163 | 16: 32472255 | 75166 | 16: 32570779 | 75268 | 16: 35045999 | 75271 | 16: 35171790 |
| 75403 | 16: 38151804 | 75444 | 16: 39447477 | 75514 | 16: 41852637 | 75544 | 16: 41964938 | 75601 | 16: 44301023 |
| 75602 | 16: 44309062 | 75603 | 16: 44341029 | 75675 | 16: 46932908 | 75678 | 16: 47002315 | 75805 | 16: 49746045 |
| 75821 | 16: 50190328 | 75822 | 16: 50240719 | 75839 | 16: 50745473 | 75941 | 16: 53280547 | 75975 | 16: 54055090 |
| 75976 | 16: 54077469 | 75987 | 16: 54386293 | 76037 | 16: 55777077 | 76379 | 16: 63448062 | 76420 | 16: 64372019 |
| 76474 | 16: 65163914 | 76705 | 16: 70129856 | 76767 | 16: 71527211 | 76903 | 16: 74041271 | 76913 | 16: 74276107 |
| 76922 | 16: 74520515 | 76932 | 16: 74777059 | 76992 | 16: 75746134 | 77018 | 16: 76272938 | 77019 | 16: 76273016 |
| 77054 | 16: 77113967 | 77055 | 16: 77114869 | 77056 | 16: 77115406 | 77057 | 16: 77115489 | 77058 | 16: 77115511 |
| 77059 | 16: 77115516 | 77060 | 16: 77117453 | 77061 | 16: 77124198 | 77227 | 16: 80002018 | 77342 | 17: 2538151 |
| 77417 | 17: 4163909 | 77468 | 17: 5077558 | 77480 | 17: 5375823 | 77508 | 17: 5999177 | 77613 | 17: 8012177 |
| 77632 | 17: 8453908 | 77860 | 17: 14044398 | 77915 | 17: 14966600 | 78029 | 17: 17475831 | 78037 | 17: 17737256 |
| 78038 | 17: 17740826 | 78097 | 17: 19033632 | 78335 | 17: 26269335 | 78410 | 17: 28015501 | 78497 | 17: 30487913 |
| 78560 | 17: 31986086 | 78574 | 17: 32403842 | 78580 | 17: 32489547 | 78638 | 17: 33714773 | 78647 | 17: 33972684 |
| 78649 | 17: 34035563 | 78655 | 17: 34228544 | 78680 | 17: 34921588 | 78708 | 17: 35827456 | 78896 | 17: 40827993 |
| 79097 | 17: 45794152 | 79104 | 17: 45901126 | 79186 | 17: 47856402 | 79401 | 17: 52535678 | 79405 | 17: 52588133 |
| 79527 | 17: 55767778 | 79528 | 17: 55769246 | 79534 | 17: 55892881 | 79577 | 17: 57167393 | 79582 | 17: 57279391 |
| 79748 | 17: 60357076 | 79754 | 17: 60395994 | 79778 | 17: 60781502 | 79835 | 17: 61664121 | 79837 | 17: 61736121 |
| 79860 | 17: 62385783 | 79871 | 17: 62699773 | 79873 | 17: 62731132 | 79876 | 17: 62794522 | 79877 | 17: 62820495 |
| 79916 | 17: 63612425 | 79928 | 17: 63638557 | 79962 | 17: 64318575 | 80008 | 17: 65293792 | 80030 | 17: 65746912 |
| 80050 | 17: 66155014 | 80061 | 17: 66243332 | 80076 | 17: 66473136 | 80079 | 17: 66581559 | 80083 | 17: 66695254 |
| 80109 | 17: 67239268 | 80249 | 17: 70356568 | 80285 | 17: 70987613 | 80303 | 17: 71333851 | 80363 | 17: 72400135 |
| 80592 | 18: 3769740 | 80636 | 18: 4682446 | 80731 | 18: 6411919 | 80773 | 18: 7301014 | 80800 | 18: 7879780 |
| 80807 | 18: 8137616 | 80811 | 18: 8163590 | 80812 | 18: 8166965 | 80813 | 18: 8169505 | 80848 | 18: 8910517 |
| 80881 | 18: 9393342 | 80887 | 18: 9441291 | 80933 | 18: 10341158 | 80973 | 18: 11082461 | 81013 | 18: 11950846 |
| 81055 | 18: 13195401 | 81339 | 18: 19844264 | 81450 | 18: 22200958 | 81455 | 18: 22265614 | 81495 | 18: 23178646 |
| 81501 | 18: 23288724 | 81645 | 18: 26295597 | 81707 | 18: 27873728 | 81901 | 18: 33007379 | 81923 | 18: 33448672 |
| 81932 | 18: 33625498 | 81933 | 18: 33654608 | 81936 | 18: 33679712 | 81937 | 18: 33689480 | 81939 | 18: 33731026 |
| 81940 | 18: 33765822 | 81945 | 18: 33836053 | 81946 | 18: 33868226 | 82061 | 18: 36768945 | 82065 | 18: 36877351 |
| 82083 | 18: 37328492 | 82099 | 18: 37566458 | 82114 | 18: 37873077 | 82177 | 18: 39306524 | 82178 | 18: 39344592 |
| 82179 | 18: 39385561 | 82181 | 18: 39440689 | 82184 | 18: 39467461 | 82186 | 18: 39488321 | 82190 | 18: 39592965 |
| 82370 | 18: 43157279 | 82379 | 18: 43316323 | 82382 | 18: 43368967 | 82508 | 18: 46424023 | 82580 | 18: 46784427 |
| 82583 | 18: 46865748 | 82670 | 18: 48351248 | 82698 | 18: 49004338 | 82732 | 18: 49949153 | 82736 | 18: 49996660 |
| 82831 | 18: 52094939 | 82855 | 18: 52727408 | 82885 | 18: 53596284 | 82887 | 18: 53619711 | 82909 | 18: 53938427 |
| 82919 | 18: 54093736 | 82940 | 18: 54546398 | 82942 | 18: 54586408 | 82983 | 18: 55141854 | 82984 | 18: 55173246 |
| 83013 | 18: 55944026 | 83110 | 18: 57018026 | 83153 | 18: 57798250 | 83172 | 18: 57998293 | 83257 | 18: 59897080 |
| 83291 | 18: 61008032 | 83319 | 18: 61724391 | 83595 | 19: 414615 | 84130 | 19: 13223983 | 84131 | 19: 13225470 |
| 84175 | 19: 13777565 | 84315 | 19: 16698614 | 84335 | 19: 16990785 | 84387 | 19: 17914841 | 84393 | 19: 18036338 |
| 84409 | 19: 18449930 | 84457 | 19: 19539554 | 84577 | 19: 21388744 | 84702 | 19: 23904908 | 84703 | 19: 23933777 |
| 84742 | 19: 24383348 | 84781 | 19: 24846129 | 84846 | 19: 25706233 | 85007 | 19: 28109982 | 85011 | 19: 28147842 |
| 85061 | 19: 29186854 | 85119 | 19: 30424898 | 85120 | 19: 30433795 | 85121 | 19: 30435544 | 85178 | 19: 31844296 |
| 85211 | 19: 32510963 | 85345 | 19: 34890238 | 85357 | 19: 34981210 | 85366 | 19: 35152019 | 85409 | 19: 36144917 |
| 85481 | 19: 37101024 | 85602 | 19: 40338519 | 85855 | 19: 45407828 | 85921 | 19: 46516531 | 85941 | 19: 47101703 |
| 85951 | 19: 47151917 | 85971 | 19: 47631810 | 86015 | 19: 48407091 | 86087 | 19: 49926869 | 86124 | 19: 50694652 |
| 86143 | 19: 50789408 | 86202 | 19: 51325894 | 86259 | 19: 52650212 | 86283 | 19: 53097970 | 86292 | 19: 53198253 |
| 86293 | 19: 53206247 | 86319 | 19: 53762930 | 86348 | 19: 54137367 | 86384 | 19: 54758347 | 86416 | 19: 55474861 |
| 86488 | 19: 56006886 | 86522 | 19: 56138971 | 86531 | 19: 56253321 | 86568 | 19: 56726108 | 86580 | 19: 56980273 |
| 86813 | 19: 61050416 | 86822 | 19: 61175458 | 86893 | 19: 62160575 | 86898 | 19: 62249612 | 86900 | 19: 62272214 |
| 86997 | 20: 909954 | 87043 | 20: 1738655 | 87052 | 20: 1886807 | 87083 | 20: 2362867 | 87101 | 20: 2682912 |
| 87142 | 20: 3442790 | 87149 | 20: 3585646 | 87174 | 20: 4263007 | 87176 | 20: 4284310 | 87177 | 20: 4331799 |
| 87190 | 20: 4516902 | 87193 | 20: 4587717 | 87194 | 20: 4587833 | 87195 | 20: 4587839 | 87196 | 20: 4588506 |
| 87197 | 20: 4588549 | 87198 | 20: 4588618 | 87199 | 20: 4588637 | 87200 | 20: 4588654 | 87203 | 20: 4620871 |
| 87204 | 20: 4621075 | 87205 | 20: 4621110 | 87206 | 20: 4621122 | 87207 | 20: 4622072 | 87208 | 20: 4622166 |
| 87209 | 20: 4622378 | 87210 | 20: 4622780 | 87211 | 20: 4622804 | 87212 | 20: 4622902 | 87213 | 20: 4623044 |
| 87214 | 20: 4623114 | 87215 | 20: 4623572 | 87216 | 20: 4624342 | 87217 | 20: 4624492 | 87218 | 20: 4624645 |
| 87219 | 20: 4625064 | 87220 | 20: 4625438 | 87221 | 20: 4626150 | 87222 | 20: 4626194 | 87223 | 20: 4626939 |
| 87224 | 20: 4627062 | 87225 | 20: 4628179 | 87226 | 20: 4628239 | 87227 | 20: 4629196 | 87228 | 20: 4629784 |
| 87229 | 20: 4630247 | 87230 | 20: 4630301 | 87231 | 20: 4630500 | 87232 | 20: 4630563 | 87233 | 20: 4630761 |
| 87234 | 20: 4630879 | 87235 | 20: 4630925 | 87236 | 20: 4631015 | 87237 | 20: 4631031 | 87238 | 20: 4631157 |
| 87239 | 20: 4631655 | 87240 | 20: 4632251 | 87241 | 20: 4632285 | 87242 | 20: 4632908 | 87243 | 20: 4633050 |
| 87244 | 20: 4633087 | 87245 | 20: 4633727 | 87246 | 20: 4633738 | 87247 | 20: 4633830 | 87248 | 20: 4634166 |
| 87249 | 20: 4634320 | 87250 | 20: 4634619 | 87251 | 20: 4634657 | 87252 | 20: 4635386 | 87253 | 20: 4635483 |
| 87254 | 20: 4635990 | 87255 | 20: 4637075 | 87256 | 20: 4637125 | 87257 | 20: 4638775 | 87258 | 20: 4638910 |
| 87259 | 20: 4639096 | 87260 | 20: 4640118 | 87261 | 20: 4640234 | 87262 | 20: 4644059 | 87263 | 20: 4644327 |
| 87264 | 20: 4652157 | 87265 | 20: 4654656 | 87267 | 20: 4658089 | 87268 | 20: 4658953 | 87269 | 20: 4661324 |
| 87270 | 20: 4665088 | 87271 | 20: 4666775 | 87272 | 20: 4667789 | 87275 | 20: 4685771 | 87277 | 20: 4690335 |
| 87278 | 20: 4690908 | 87279 | 20: 4691249 | 87280 | 20: 4697066 | 87281 | 20: 4697631 | 87282 | 20: 4701728 |
| 87283 | 20: 4701832 | 87284 | 20: 4708012 | 87285 | 20: 4709731 | 87286 | 20: 4710087 | 87287 | 20: 4712587 |
| 87288 | 20: 4713160 | 87289 | 20: 4715176 | 87290 | 20: 4716043 | 87291 | 20: 4726582 | 87292 | 20: 4728514 |
| 87293 | 20: 4730765 | 87294 | 20: 4732330 | 87296 | 20: 4736819 | 87297 | 20: 4741050 | 87298 | 20: 4750279 |
| 87299 | 20: 4751162 | 87300 | 20: 4752621 | 87301 | 20: 4753160 | 87302 | 20: 4757934 | 87303 | 20: 4758722 |
| 87304 | 20: 4761361 | 87305 | 20: 4763955 | 87306 | 20: 4770162 | 87309 | 20: 4797785 | 87310 | 20: 4805282 |
| 87311 | 20: 4812157 | 87312 | 20: 4819449 | 87314 | 20: 4838678 | 87315 | 20: 4841899 | 87316 | 20: 4842019 |
| 87317 | 20: 4846348 | 87318 | 20: 4850816 | 87319 | 20: 4851612 | 87320 | 20: 4853215 | 87321 | 20: 4854589 |
| 87322 | 20: 4863651 | 87323 | 20: 4873619 | 87324 | 20: 4873645 | 87325 | 20: 4873658 | 87326 | 20: 4873667 |
| 87327 | 20: 4880129 | 87328 | 20: 4885163 | 87329 | 20: 4922714 | 87330 | 20: 4927232 | 87332 | 20: 4928741 |
| 87333 | 20: 4936772 | 87334 | 20: 4937498 | 87335 | 20: 4938011 | 87336 | 20: 4938521 | 87337 | 20: 4938780 |
| 87338 | 20: 4938885 | 87339 | 20: 4939294 | 87340 | 20: 4940782 | 87341 | 20: 4940805 | 87342 | 20: 4944507 |
| 87343 | 20: 4947601 | 87344 | 20: 4949204 | 87345 | 20: 4964757 | 87346 | 20: 4965179 | 87347 | 20: 4965760 |
| 87348 | 20: 4969010 | 87349 | 20: 4971347 | 87350 | 20: 4971831 | 87351 | 20: 4972329 | 87352 | 20: 4974179 |
| 87354 | 20: 4975337 | 87355 | 20: 4978069 | 87356 | 20: 4978213 | 87357 | 20: 4978475 | 87359 | 20: 4982228 |
| 87360 | 20: 4984846 | 87361 | 20: 4985523 | 87362 | 20: 4986398 | 87363 | 20: 4988142 | 87364 | 20: 4990340 |
| 87365 | 20: 4991225 | 87367 | 20: 4992704 | 87368 | 20: 4992712 | 87369 | 20: 4993558 | 87370 | 20: 4993669 |
| 87371 | 20: 4993957 | 87373 | 20: 4999299 | 87374 | 20: 4999988 | 87376 | 20: 5002715 | 87377 | 20: 5003850 |
| 87378 | 20: 5004638 | 87379 | 20: 5008738 | 87380 | 20: 5009598 | 87383 | 20: 5040971 | 87385 | 20: 5045805 |
| 87386 | 20: 5049196 | 87387 | 20: 5054905 | 87388 | 20: 5055091 | 87389 | 20: 5055857 | 87390 | 20: 5058209 |
| 87391 | 20: 5070761 | 87392 | 20: 5074751 | 87393 | 20: 5075106 | 87394 | 20: 5076823 | 87395 | 20: 5079687 |
| 87397 | 20: 5081368 | 87399 | 20: 5082509 | 87400 | 20: 5107041 | 87402 | 20: 5129728 | 87415 | 20: 5282517 |
| 87429 | 20: 5485968 | 87434 | 20: 5633232 | 87435 | 20: 5665364 | 87436 | 20: 5695030 | 87438 | 20: 5754805 |
| 87439 | 20: 5796361 | 87449 | 20: 6060819 | 87450 | 20: 6062848 | 87451 | 20: 6063007 | 87452 | 20: 6063251 |
| 87453 | 20: 6063553 | 87454 | 20: 6063862 | 87455 | 20: 6063984 | 87456 | 20: 6064358 | 87457 | 20: 6064442 |
| 87458 | 20: 6066220 | 87459 | 20: 6066792 | 87460 | 20: 6066880 | 87461 | 20: 6067197 | 87462 | 20: 6069267 |
| 87463 | 20: 6069326 | 87464 | 20: 6069531 | 87465 | 20: 6072588 | 87466 | 20: 6073779 | 87467 | 20: 6076915 |
| 87470 | 20: 6090071 | 87477 | 20: 6208301 | 87484 | 20: 6325657 | 87488 | 20: 6444472 | 87490 | 20: 6526713 |
| 87509 | 20: 6914755 | 87513 | 20: 7017878 | 87516 | 20: 7049088 | 87519 | 20: 7079494 | 87521 | 20: 7089351 |
| 87537 | 20: 7437979 | 87539 | 20: 7473329 | 87548 | 20: 7706288 | 87550 | 20: 7739561 | 87557 | 20: 7958728 |
| 87559 | 20: 7978859 | 87564 | 20: 8099584 | 87566 | 20: 8120294 | 87570 | 20: 8190372 | 87573 | 20: 8244125 |
| 87599 | 20: 8976316 | 87608 | 20: 9074647 | 87609 | 20: 9107005 | 87662 | 20: 10266518 | 87686 | 20: 10946439 |
| 87698 | 20: 11213830 | 87702 | 20: 11274635 | 87711 | 20: 11438125 | 87734 | 20: 11948110 | 87736 | 20: 12001911 |
| 87747 | 20: 12228546 | 87750 | 20: 12265622 | 87755 | 20: 12409400 | 87758 | 20: 12445726 | 87824 | 20: 13931338 |
| 87826 | 20: 13967565 | 87829 | 20: 14050805 | 87830 | 20: 14120849 | 87885 | 20: 16058541 | 87923 | 20: 17088438 |
| 87931 | 20: 17251657 | 88103 | 20: 21950333 | 88188 | 20: 22481257 | 88190 | 20: 22578241 | 88220 | 20: 23180410 |
| 88233 | 20: 23492042 | 88239 | 20: 23575761 | 88241 | 20: 23623587 | 88244 | 20: 23695033 | 88334 | 20: 25681663 |
| 88342 | 20: 26011480 | 88352 | 20: 26319951 | 88372 | 20: 26721455 | 88379 | 20: 26929602 | 88486 | 20: 29337360 |
| 88501 | 20: 29797692 | 88594 | 20: 32271969 | 88629 | 20: 33323766 | 88635 | 20: 33477656 | 88636 | 20: 33482995 |
| 88651 | 20: 33788019 | 88843 | 20: 38077292 | 88880 | 20: 39076620 | 88893 | 20: 39383171 | 88900 | 20: 39458208 |
| 88901 | 20: 39462501 | 88903 | 20: 39469722 | 88906 | 20: 39485628 | 88970 | 20: 41005245 | 88987 | 20: 41215061 |
| 89024 | 20: 41990636 | 89201 | 20: 46912182 | 89292 | 20: 49691751 | 89327 | 20: 50794831 | 89331 | 20: 50908612 |
| 89334 | 20: 50953384 | 89388 | 20: 52906490 | 89424 | 20: 53680094 | 89427 | 20: 53779182 | 89490 | 20: 55580535 |
| 89491 | 20: 55615423 | 89501 | 20: 55920256 | 89504 | 20: 55944017 | 89603 | 20: 58030736 | 89607 | 20: 58078386 |
| 89748 | 20: 61558977 | 89850 | 20: 63825165 | 89852 | 20: 63847390 | 89855 | 20: 63882214 | 89947 | 20: 65988032 |
| 89964 | 20: 66468697 | 90010 | 20: 67443129 | 90042 | 20: 68014966 | 90105 | 20: 69097447 | 90110 | 20: 69174801 |
| 90114 | 20: 69288227 | 90116 | 20: 69317277 | 90161 | 20: 70296851 | 90314 | 21: 2109801 | 90321 | 21: 2416354 |
| 90340 | 21: 2937636 | 90422 | 21: 5168513 | 90441 | 21: 5776117 | 90447 | 21: 5802937 | 90449 | 21: 5865604 |
| 90452 | 21: 5932063 | 90531 | 21: 8044281 | 90549 | 21: 8125084 | 90577 | 21: 8739099 | 90673 | 21: 10967078 |
| 90676 | 21: 11012534 | 90870 | 21: 15628004 | 90871 | 21: 15629471 | 90872 | 21: 15634222 | 90873 | 21: 15635039 |
| 90874 | 21: 15636617 | 90875 | 21: 15640534 | 90876 | 21: 15644415 | 90877 | 21: 15648341 | 90878 | 21: 15652428 |
| 90879 | 21: 15656729 | 90964 | 21: 17632245 | 90983 | 21: 18074742 | 90987 | 21: 18139041 | 91008 | 21: 18709801 |
| 91009 | 21: 18745959 | 91041 | 21: 19240261 | 91119 | 21: 20568083 | 91133 | 21: 20667605 | 91163 | 21: 21254778 |
| 91164 | 21: 21278489 | 91244 | 21: 22980503 | 91310 | 21: 24894924 | 91377 | 21: 26762300 | 91417 | 21: 27993786 |
| 91435 | 21: 28714736 | 91624 | 21: 33373608 | 91661 | 21: 33774652 | 91702 | 21: 34416654 | 91768 | 21: 35722374 |
| 91868 | 21: 38504846 | 91877 | 21: 38644972 | 91941 | 21: 40541468 | 91985 | 21: 41630642 | 92011 | 21: 42193358 |
| 92020 | 21: 42461759 | 92029 | 21: 42678313 | 92115 | 21: 44563811 | 92120 | 21: 44677322 | 92171 | 21: 46302745 |
| 92332 | 21: 49387921 | 92335 | 21: 49487176 | 92397 | 21: 51289914 | 92407 | 21: 51606765 | 92484 | 21: 53761679 |
| 92488 | 21: 53814553 | 92726 | 21: 57261794 | 92848 | 21: 59894217 | 92855 | 21: 59981922 | 92967 | 21: 62224560 |
| 93037 | 21: 64128429 | 93155 | 21: 66126088 | 93201 | 21: 67026936 | 93296 | 21: 69054783 | 93298 | 21: 69111628 |
| 93301 | 21: 69165607 | 93601 | 22: 5786344 | 93653 | 22: 6683071 | 93663 | 22: 6874059 | 93668 | 22: 6973667 |
| 93683 | 22: 7347580 | 93785 | 22: 10006376 | 93807 | 22: 10285746 | 93819 | 22: 10597656 | 93821 | 22: 10648705 |
| 93829 | 22: 10879669 | 93832 | 22: 10961055 | 93894 | 22: 12251220 | 94047 | 22: 16130993 | 94110 | 22: 17624753 |
| 94177 | 22: 19212118 | 94255 | 22: 21157473 | 94279 | 22: 21651340 | 94283 | 22: 21690013 | 94326 | 22: 22930737 |
| 94380 | 22: 24606457 | 94410 | 22: 25532201 | 94504 | 22: 28088504 | 94526 | 22: 28449795 | 94614 | 22: 30572708 |
| 94616 | 22: 30617079 | 94621 | 22: 30762175 | 94689 | 22: 32709942 | 94691 | 22: 32740432 | 94806 | 22: 35660903 |
| 94908 | 22: 38005262 | 94968 | 22: 39268641 | 95000 | 22: 39706378 | 95013 | 22: 39906000 | 95037 | 22: 40607655 |
| 95117 | 22: 42731135 | 95121 | 22: 42898400 | 95122 | 22: 42936001 | 95284 | 22: 46414503 | 95300 | 22: 46810720 |
| 95307 | 22: 46910055 | 95353 | 22: 47726147 | 95583 | 22: 53279742 | 95622 | 22: 54013319 | 95666 | 22: 54873498 |
| 95669 | 22: 54947766 | 95676 | 22: 55073957 | 95716 | 22: 55626326 | 95781 | 22: 56400223 | 95784 | 22: 56416067 |
| 95785 | 22: 56419658 | 95786 | 22: 56420213 | 95809 | 22: 56829490 | 95908 | 22: 58476458 | 95913 | 22: 58524949 |
| 95955 | 22: 59413554 | 96087 | 23: 2001057 | 96090 | 23: 2028074 | 96118 | 23: 2653886 | 96121 | 23: 2672734 |
| 96124 | 23: 2696666 | 96171 | 23: 3661084 | 96177 | 23: 3744075 | 96232 | 23: 4789896 | 96422 | 23: 8682465 |
| 96482 | 23: 10137524 | 96546 | 23: 11326784 | 96550 | 23: 11418685 | 96552 | 23: 11493234 | 96644 | 23: 13147408 |
| 96647 | 23: 13234890 | 96777 | 23: 15576268 | 96872 | 23: 17807855 | 96930 | 23: 19250498 | 96931 | 23: 19271779 |
| 96933 | 23: 19307959 | 96941 | 23: 19459539 | 96947 | 23: 19483200 | 96959 | 23: 19635143 | 96965 | 23: 19711944 |
| 96996 | 23: 20201294 | 97043 | 23: 21249255 | 97051 | 23: 21366692 | 97071 | 23: 21956033 | 97096 | 23: 22583782 |
| 97278 | 23: 25346058 | 97357 | 23: 27009758 | 97451 | 23: 27999156 | 97501 | 23: 28242781 | 97780 | 23: 32969917 |
| 97828 | 23: 34109165 | 97943 | 23: 37547849 | 97981 | 23: 38637301 | 98382 | 23: 45418705 | 98389 | 23: 45533685 |
| 98404 | 23: 46021030 | 98438 | 23: 46778964 | 98556 | 23: 48912747 | 98631 | 23: 50033982 | 98724 | 23: 51773706 |
| 98783 | 24: 520222 | 98832 | 24: 1526726 | 98846 | 24: 1793795 | 98892 | 24: 2708333 | 98894 | 24: 2766149 |
| 98913 | 24: 3184889 | 98915 | 24: 3208218 | 98935 | 24: 3712541 | 98952 | 24: 4048939 | 98964 | 24: 4188152 |
| 98982 | 24: 4612976 | 98994 | 24: 5077959 | 99037 | 24: 6230000 | 99125 | 24: 8320822 | 99297 | 24: 10714691 |
| 99353 | 24: 12020111 | 99356 | 24: 12116867 | 99361 | 24: 12381665 | 99369 | 24: 12616739 | 99402 | 24: 13417942 |
| 99414 | 24: 13675366 | 99451 | 24: 14795404 | 99594 | 24: 19151288 | 99665 | 24: 20730048 | 99691 | 24: 21214702 |
| 99863 | 24: 25023821 | 99870 | 24: 25259209 | 99898 | 24: 25782545 | 99909 | 24: 25992482 | 100009 | 24: 28570245 |
| 100053 | 24: 29754026 | 100059 | 24: 29814575 | 100120 | 24: 31415877 | 100199 | 24: 32894188 | 100245 | 24: 34122422 |
| 100257 | 24: 34395443 | 100267 | 24: 34719362 | 100285 | 24: 35185903 | 100316 | 24: 35736460 | 100323 | 24: 35860154 |
| 100338 | 24: 36195505 | 100358 | 24: 37011897 | 100362 | 24: 37166678 | 100364 | 24: 37203930 | 100429 | 24: 39199046 |
| 100782 | 24: 46297155 | 100791 | 24: 46409035 | 100919 | 24: 49840655 | 100982 | 24: 51488828 | 101005 | 24: 51881818 |
| 101010 | 24: 51956661 | 101014 | 24: 52037619 | 101245 | 24: 57052356 | 101347 | 24: 59016719 | 101349 | 24: 59106967 |
| 101394 | 24: 60056629 | 101395 | 24: 60093951 | 101469 | 24: 61534805 | 101551 | 25: 1163011 | 101605 | 25: 1918071 |
| 101614 | 25: 2105032 | 101618 | 25: 2179579 | 101802 | 25: 3997589 | 101849 | 25: 4921820 | 101868 | 25: 5339582 |
| 101958 | 25: 6766372 | 101966 | 25: 6952022 | 102005 | 25: 7896632 | 102011 | 25: 7949848 | 102018 | 25: 8118349 |
| 102131 | 25: 10832103 | 102149 | 25: 11113745 | 102307 | 25: 14593246 | 102318 | 25: 14917112 | 102323 | 25: 15012071 |
| 102329 | 25: 15106926 | 102356 | 25: 15663604 | 102365 | 25: 15912161 | 102380 | 25: 16148690 | 102399 | 25: 16456322 |
| 102413 | 25: 16841496 | 102459 | 25: 17915319 | 102460 | 25: 17952726 | 102462 | 25: 18009869 | 102490 | 25: 18825304 |
| 102523 | 25: 19553139 | 102596 | 25: 21400170 | 102612 | 25: 21756847 | 102647 | 25: 22463379 | 102656 | 25: 22697194 |
| 102676 | 25: 23209925 | 102679 | 25: 23393931 | 102695 | 25: 23751702 | 102713 | 25: 24073747 | 102724 | 25: 24300333 |
| 102732 | 25: 24496560 | 102767 | 25: 25119747 | 102774 | 25: 25175370 | 102851 | 25: 26611224 | 102875 | 25: 27289405 |
| 102971 | 25: 29170307 | 102973 | 25: 29196073 | 102976 | 25: 29227741 | 103031 | 25: 30198863 | 103036 | 25: 30292715 |
| 103045 | 25: 30543167 | 103047 | 25: 30590381 | 103165 | 25: 33056358 | 103226 | 25: 34314708 | 103243 | 25: 34536825 |
| 103265 | 25: 34878010 | 103273 | 25: 35037480 | 103294 | 25: 35416991 | 103369 | 25: 36281889 | 103468 | 25: 37785020 |
| 103522 | 25: 38925016 | 103562 | 25: 39799663 | 103598 | 25: 40282487 | 103603 | 25: 40362153 | 103698 | 25: 42167568 |
| 103761 | 26: 1999995 | 103766 | 26: 2270743 | 103811 | 26: 2874049 | 103964 | 26: 7323964 | 104015 | 26: 8514472 |
| 104061 | 26: 9801561 | 104175 | 26: 12542210 | 104214 | 26: 13232302 | 104243 | 26: 13935413 | 104244 | 26: 13974845 |
| 104248 | 26: 14058998 | 104265 | 26: 14560437 | 104266 | 26: 14562388 | 104420 | 26: 18423545 | 104469 | 26: 19395441 |
| 104485 | 26: 19632302 | 104679 | 26: 24855737 | 104714 | 26: 25459976 | 104715 | 26: 25463718 | 104716 | 26: 25465826 |
| 104717 | 26: 25467173 | 104781 | 26: 26561752 | 104860 | 26: 28504622 | 104925 | 26: 30397842 | 104932 | 26: 30643098 |
| 104935 | 26: 30753391 | 104947 | 26: 31085230 | 105378 | 26: 41389219 | 105381 | 26: 41451309 | 105394 | 26: 41900876 |
| 105445 | 26: 42982393 | 105499 | 26: 44238502 | 105742 | 26: 49454109 | 105754 | 26: 49637614 | 105813 | 26: 51001537 |
| 105824 | 26: 51205579 | 105851 | 26: 51911589 | 105854 | 26: 51990348 | 105886 | 27: 1292211 | 105932 | 27: 2099758 |
| 105955 | 27: 2545985 | 106036 | 27: 4477596 | 106056 | 27: 5041534 | 106064 | 27: 5224821 | 106086 | 27: 5489950 |
| 106115 | 27: 6134492 | 106135 | 27: 7463182 | 106203 | 27: 8803096 | 106277 | 27: 10171403 | 106312 | 27: 11344637 |
| 106334 | 27: 11976000 | 106374 | 27: 12998392 | 106452 | 27: 13945896 | 106510 | 27: 15278766 | 106547 | 27: 16013203 |
| 106553 | 27: 16091265 | 106558 | 27: 16161541 | 106608 | 27: 16989882 | 106617 | 27: 17229318 | 106655 | 27: 18039702 |
| 106668 | 27: 18444933 | 106672 | 27: 18549440 | 106674 | 27: 18561932 | 106695 | 27: 18999723 | 106753 | 27: 20126660 |
| 106800 | 27: 21693284 | 106837 | 27: 22606696 | 106839 | 27: 22612038 | 106840 | 27: 22625012 | 106841 | 27: 22635635 |
| 106851 | 27: 22872901 | 106892 | 27: 23844184 | 106958 | 27: 25097722 | 106963 | 27: 25227473 | 107031 | 27: 26703643 |
| 107032 | 27: 26707702 | 107033 | 27: 26709108 | 107034 | 27: 26717112 | 107035 | 27: 26752145 | 107037 | 27: 26821155 |
| 107116 | 27: 28602334 | 107142 | 27: 29184261 | 107215 | 27: 30821123 | 107219 | 27: 30967912 | 107275 | 27: 31920386 |
| 107418 | 27: 35019984 | 107424 | 27: 35045715 | 107434 | 27: 35072059 | 107439 | 27: 35098802 | 107452 | 27: 35316815 |
| 107478 | 27: 35710124 | 107512 | 27: 36366267 | 107541 | 27: 37168180 | 107629 | 27: 38769358 | 107630 | 27: 38784049 |
| 107632 | 27: 38825952 | 107648 | 27: 39079734 | 107650 | 27: 39140091 | 107659 | 27: 39414442 | 107684 | 27: 40138920 |
| 107723 | 27: 41177956 | 107855 | 27: 44120630 | 107940 | 28: 1026816 | 107943 | 28: 1063432 | 107978 | 28: 1669722 |
| 107979 | 28: 1676667 | 107980 | 28: 1688640 | 108035 | 28: 3310204 | 108064 | 28: 4807450 | 108072 | 28: 5021515 |
| 108093 | 28: 5430772 | 108094 | 28: 5471888 | 108109 | 28: 5837959 | 108120 | 28: 5926093 | 108142 | 28: 6534383 |
| 108248 | 28: 9052050 | 108503 | 28: 15552135 | 108505 | 28: 15620196 | 108621 | 28: 17961688 | 108634 | 28: 18086730 |
| 108720 | 28: 20471303 | 108761 | 28: 21757902 | 108775 | 28: 22091224 | 108882 | 28: 24568402 | 108911 | 28: 25196334 |
| 108950 | 28: 26054541 | 108997 | 28: 26764566 | 109005 | 28: 26976598 | 109043 | 28: 27784145 | 109225 | 28: 31516271 |
| 109243 | 28: 31960964 | 109334 | 28: 33369552 | 109355 | 28: 33823084 | 109363 | 28: 34002468 | 109397 | 28: 34663948 |
| 109420 | 28: 34999208 | 109504 | 28: 37068693 | 109511 | 28: 37195142 | 109515 | 28: 37311281 | 109519 | 28: 37446774 |
| 109549 | 28: 37921033 | 109557 | 28: 38098897 | 109621 | 28: 39341830 | 109741 | 28: 40986070 | 109744 | 28: 41014073 |
| 109881 | 28: 43434313 | 109929 | 28: 44311425 | 109935 | 28: 44453574 | 109945 | 28: 44780535 | 109958 | 28: 44981684 |
| 109988 | 28: 45527255 | 110035 | 29: 1014606 | 110080 | 29: 1997329 | 110082 | 29: 2117573 | 110221 | 29: 5006464 |
| 110233 | 29: 5358686 | 110234 | 29: 5358699 | 110248 | 29: 5885429 | 110252 | 29: 5979872 | 110429 | 29: 10496324 |
| 110449 | 29: 11034651 | 110590 | 29: 14470262 | 110606 | 29: 14866248 | 110721 | 29: 17901471 | 110722 | 29: 17951025 |
| 110723 | 29: 17955354 | 110769 | 29: 19855058 | 110770 | 29: 19861520 | 110771 | 29: 19862538 | 110985 | 29: 25169363 |
| 110987 | 29: 25193462 | 111011 | 29: 25759092 | 111063 | 29: 27485292 | 111143 | 29: 28949555 | 111155 | 29: 29347332 |
| 111211 | 29: 30136039 | 111212 | 29: 30157180 | 111221 | 29: 30347114 | 111336 | 29: 32940189 | 111340 | 29: 33039863 |
| 111384 | 29: 34046874 | 111390 | 29: 34239637 | 111402 | 29: 34523872 | 111422 | 29: 34808241 | 111479 | 29: 35951280 |
| 111481 | 29: 35999607 | 111550 | 29: 37304798 | 111551 | 29: 37320062 | 111658 | 29: 40811556 | 111659 | 29: 40812741 |
| 111660 | 29: 40813920 | 111661 | 29: 40818497 | 111662 | 29: 40820427 | 111663 | 29: 40820963 | 111664 | 29: 40821065 |
| 111666 | 29: 40825872 | 111806 | 29: 43407527 | 111808 | 29: 43424442 | 111892 | 29: 44721351 | 111901 | 29: 45041058 |
| 111904 | 29: 45172298 | 111928 | 29: 45712362 | 111979 | 29: 46833503 | 111989 | 29: 46997866 | 111995 | 29: 47125684 |
| 111996 | 29: 47150455 | 112100 | 29: 49234278 | 112124 | 29: 49722188 | 112140 | 29: 50217955 | 112141 | 29: 50260533; |

and
a screening method of the sixth marker panel comprises the following steps: allowing genotyping results of the BovineHD chip of Illumina and the GGP Bovine 100K chip of Neogen in the Huaxi cattle population to have quality control at a minor allele frequency (MAF) of greater than 0.1, a genotype deletion rate of less than 10%, and a P value of Hardy-Weinberg equilibrium (HWE) testing of 1×10⁻⁶, and deleting invalid sites while retaining effective information marker sites in the Huaxi cattle population; and
the sites of the seventh marker panel are shown in the following table:

| NO. | Position | NO. | Position | NO. | Position | NO. | Position | NO. | Position |
|---|---|---|---|---|---|---|---|---|---|
| 1 | 1: 198611 | 2 | 1: 354105 | 3 | 1: 413686 | 4 | 1: 503436 | 5 | 1: 539006 |
| 6 | 1: 561519 | 8 | 1: 752778 | 114 | 1: 2548682 | 115 | 1: 2645413 | 116 | 1: 2738165 |
| 120 | 1: 2859590 | 248 | 1: 5217586 | 249 | 1: 5308367 | 394 | 1: 8387543 | 518 | 1: 11192831 |
| 542 | 1: 11918795 | 549 | 1: 12187898 | 551 | 1: 12295655 | 578 | 1: 13034308 | 581 | 1: 13135427 |
| 624 | 1: 13936005 | 625 | 1: 14033343 | 634 | 1: 14325639 | 689 | 1: 15749427 | 709 | 1: 16317871 |
| 793 | 1: 19108624 | 795 | 1: 19246215 | 816 | 1: 19761054 | 817 | 1: 19840115 | 906 | 1: 22225114 |
| 926 | 1: 22716338 | 927 | 1: 22813570 | 942 | 1: 23156851 | 943 | 1: 23255421 | 950 | 1: 23587167 |
| 1081 | 1: 27110041 | 1096 | 1: 27690352 | 1099 | 1: 27826641 | 1121 | 1: 28708529 | 1184 | 1: 30341410 |
| 1188 | 1: 30639309 | 1228 | 1: 31833724 | 1232 | 1: 32038632 | 1239 | 1: 32383066 | 1245 | 1: 32604779 |
| 1302 | 1: 34248126 | 1303 | 1: 34341221 | 1336 | 1: 35240562 | 1357 | 1: 35661327 | 1373 | 1: 36152445 |
| 1491 | 1: 39146665 | 1500 | 1: 39465349 | 1501 | 1: 39555945 | 1528 | 1: 40302747 | 1529 | 1: 40392357 |
| 1555 | 1: 41086735 | 1563 | 1: 41385809 | 1565 | 1: 41492237 | 1626 | 1: 42693702 | 1627 | 1: 42705366 |
| 1628 | 1: 42797258 | 1769 | 1: 45607475 | 1771 | 1: 45687245 | 1772 | 1: 45787243 | 1818 | 1: 47287965 |
| 1838 | 1: 47969845 | 1845 | 1: 48250419 | 1876 | 1: 49256468 | 1899 | 1: 50064432 | 1921 | 1: 50739583 |
| 1956 | 1: 51942050 | 1961 | 1: 52098640 | 2014 | 1: 53514840 | 2090 | 1: 54855775 | 2101 | 1: 55175239 |
| 2117 | 1: 55765709 | 2124 | 1: 55975682 | 2125 | 1: 56071064 | 2227 | 1: 58399681 | 2228 | 1: 58497604 |
| 2264 | 1: 59389492 | 2269 | 1: 59650598 | 2357 | 1: 61707467 | 2364 | 1: 61908265 | 2533 | 1: 65639354 |
| 2534 | 1: 65691897 | 2542 | 1: 65975858 | 2553 | 1: 66261981 | 2554 | 1: 66349787 | 2575 | 1: 66970472 |
| 2601 | 1: 67667419 | 2612 | 1: 68106244 | 2693 | 1: 69418925 | 2702 | 1: 69664149 | 2804 | 1: 71695300 |
| 2825 | 1: 72263210 | 2865 | 1: 73046338 | 2912 | 1: 74307455 | 2913 | 1: 74402077 | 2942 | 1: 75146396 |
| 2992 | 1: 76510120 | 3001 | 1: 76727873 | 3014 | 1: 77141699 | 3091 | 1: 78826134 | 3171 | 1: 80384212 |
| 3239 | 1: 81998130 | 3282 | 1: 82911267 | 3295 | 1: 83439908 | 3307 | 1: 83899382 | 3321 | 1: 84316332 |
| 3356 | 1: 85642906 | 3372 | 1: 86015500 | 3389 | 1: 86437496 | 3390 | 1: 86526242 | 3403 | 1: 86930423 |
| 3415 | 1: 87291852 | 3447 | 1: 88278205 | 3448 | 1: 88303482 | 3449 | 1: 88477613 | 3450 | 1: 88510142 |
| 3451 | 1: 88605108 | 3480 | 1: 89106677 | 3481 | 1: 89184242 | 3605 | 1: 92127457 | 3615 | 1: 92303587 |
| 3627 | 1: 92748568 | 3631 | 1: 92911886 | 3662 | 1: 93749721 | 3788 | 1: 96724055 | 3898 | 1: 99648631 |
| 3899 | 1: 99745058 | 3907 | 1: 99964213 | 3912 | 1: 100151147 | 3951 | 1: 101402516 | 3959 | 1: 101604960 |
| 3975 | 1: 102178820 | 3985 | 1: 102499753 | 4011 | 1: 103210827 | 4043 | 1: 104445688 | 4047 | 1: 104601851 |
| 4128 | 1: 106679632 | 4215 | 1: 108995880 | 4218 | 1: 109138566 | 4223 | 1: 109344061 | 4244 | 1: 109812035 |
| 4303 | 1: 110891497 | 4316 | 1: 111431902 | 4391 | 1: 113109748 | 4461 | 1: 114671853 | 4507 | 1: 116130150 |
| 4509 | 1: 116269280 | 4510 | 1: 116368715 | 4688 | 1: 119719715 | 4739 | 1: 120951015 | 4741 | 1: 121018439 |
| 4742 | 1: 121074170 | 4743 | 1: 121144456 | 4745 | 1: 121224156 | 4749 | 1: 121384190 | 4759 | 1: 121640449 |
| 4764 | 1: 121788170 | 4774 | 1: 122051101 | 4832 | 1: 123574290 | 4833 | 1: 123657947 | 4976 | 1: 126688131 |
| 4977 | 1: 126726689 | 4978 | 1: 126823765 | 5040 | 1: 128486623 | 5043 | 1: 128606933 | 5093 | 1: 129682985 |
| 5098 | 1: 129814612 | 5099 | 1: 129913741 | 5241 | 1: 132429392 | 5247 | 1: 132635025 | 5251 | 1: 132828896 |
| 5260 | 1: 133234769 | 5284 | 1: 134043255 | 5414 | 1: 136299174 | 5432 | 1: 136970680 | 5444 | 1: 137364879 |
| 5459 | 1: 138082957 | 5460 | 1: 138180043 | 5462 | 1: 138259482 | 5464 | 1: 138404453 | 5500 | 1: 139245663 |
| 5502 | 1: 139304863 | 5503 | 1: 139403568 | 5511 | 1: 139653452 | 5578 | 1: 141255563 | 5701 | 1: 143320922 |
| 5754 | 1: 144299047 | 5884 | 1: 146674083 | 5885 | 1: 146773640 | 6509 | 1: 157687863 | 6510 | 1: 157775988 |
| 6512 | 1: 157881491 | 6523 | 1: 158316722 | 6524 | 1: 158361834 | 6525 | 1: 158460274 | 6527 | 2: 97987 |
| 6528 | 2: 156099 | 6529 | 2: 251841 | 6609 | 2: 1676588 | 6610 | 2: 1762272 | 6627 | 2: 2208427 |
| 6711 | 2: 4459297 | 6819 | 2: 6376890 | 6897 | 2: 7947030 | 6898 | 2: 7984204 | 6899 | 2: 8067862 |
| 6921 | 2: 8713754 | 6943 | 2: 9417024 | 6945 | 2: 9540631 | 6961 | 2: 9879504 | 6962 | 2: 9977673 |
| 6990 | 2: 10739801 | 6994 | 2: 11002563 | 7030 | 2: 12006500 | 7035 | 2: 12186267 | 7046 | 2: 12679896 |
| 7117 | 2: 14600625 | 7167 | 2: 15807478 | 7249 | 2: 17949516 | 7391 | 2: 20586534 | 7405 | 2: 20982246 |
| 7482 | 2: 22953223 | 7490 | 2: 23131851 | 7539 | 2: 24040103 | 7590 | 2: 25076321 | 7805 | 2: 29677272 |
| 7836 | 2: 30463341 | 7892 | 2: 31678013 | 7912 | 2: 32367398 | 7928 | 2: 32964948 | 8001 | 2: 34879480 |
| 8002 | 2: 34940227 | 8003 | 2: 35037207 | 8015 | 2: 35478468 | 8021 | 2: 35685945 | 8062 | 2: 36835120 |
| 8185 | 2: 40374618 | 8195 | 2: 40590316 | 8364 | 2: 44741204 | 8381 | 2: 45256936 | 8524 | 2: 48700283 |
| 8548 | 2: 49196955 | 8550 | 2: 49308768 | 8552 | 2: 49502134 | 8577 | 2: 50260674 | 8621 | 2: 51619705 |
| 8622 | 2: 51714357 | 8648 | 2: 52249299 | 8665 | 2: 52682136 | 8707 | 2: 53776420 | 8717 | 2: 54059294 |
| 8723 | 2: 54299723 | 8817 | 2: 56671164 | 8818 | 2: 56766656 | 8859 | 2: 57934056 | 8886 | 2: 58700978 |
| 8907 | 2: 59199942 | 8956 | 2: 60394889 | 8972 | 2: 60962014 | 9010 | 2: 61641610 | 9011 | 2: 61738618 |
| 9018 | 2: 62012037 | 9023 | 2: 62250012 | 9101 | 2: 64316198 | 9116 | 2: 64570288 | 9246 | 2: 68272329 |
| 9258 | 2: 68690400 | 9261 | 2: 68901070 | 9264 | 2: 69034698 | 9269 | 2: 69214365 | 9274 | 2: 69379425 |
| 9323 | 2: 70760400 | 9362 | 2: 71684921 | 9363 | 2: 71782525 | 9365 | 2: 71848257 | 9367 | 2: 71970045 |
| 9406 | 2: 73054461 | 9417 | 2: 73255223 | 9483 | 2: 75058828 | 9582 | 2: 77785420 | 9651 | 2: 79212662 |
| 9652 | 2: 79232935 | 9655 | 2: 79435179 | 9688 | 2: 80269896 | 9691 | 2: 80350552 | 9692 | 2: 80443906 |
| 9718 | 2: 81298125 | 9721 | 2: 81552356 | 9731 | 2: 82160162 | 9738 | 2: 82525466 | 9741 | 2: 82703708 |
| 9757 | 2: 83398924 | 9759 | 2: 83488349 | 9790 | 2: 84070506 | 9793 | 2: 84186911 | 9820 | 2: 84735988 |
| 9822 | 2: 84748768 | 9823 | 2: 84845633 | 9848 | 2: 85539387 | 9873 | 2: 86113417 | 9878 | 2: 86175227 |
| 9879 | 2: 86265979 | 9908 | 2: 86950234 | 9953 | 2: 88328898 | 9980 | 2: 88998896 | 10067 | 2: 90047120 |
| 10069 | 2: 90182742 | 10070 | 2: 90271127 | 10071 | 2: 90346277 | 10107 | 2: 91439705 | 10170 | 2: 92960490 |
| 10178 | 2: 93253701 | 10243 | 2: 94504077 | 10356 | 2: 96858239 | 10362 | 2: 97119985 | 10364 | 2: 97284470 |
| 10374 | 2: 97660903 | 10388 | 2: 97949623 | 10453 | 2: 99661757 | 10479 | 2: 100148811 | 10510 | 2: 101083832 |
| 10528 | 2: 101669777 | 10534 | 2: 101822460 | 10562 | 2: 102640055 | 10642 | 2: 104771582 | 10671 | 2: 105313365 |
| 10916 | 2: 110949976 | 10930 | 2: 111452102 | 11063 | 2: 114575713 | 11083 | 2: 115163293 | 11148 | 2: 116629100 |
| 11191 | 2: 117806168 | 11194 | 2: 117939332 | 11231 | 2: 118820218 | 11232 | 2: 118916665 | 11274 | 2: 119675734 |
| 11302 | 2: 120419474 | 11330 | 2: 121151600 | 11342 | 2: 121489660 | 11349 | 2: 121623817 | 11350 | 2: 121707578 |
| 11363 | 2: 122105560 | 11448 | 2: 124381727 | 11449 | 2: 124471534 | 11500 | 2: 125909343 | 11658 | 2: 128226965 |
| 11765 | 2: 130549076 | 11809 | 2: 131471648 | 12088 | 2: 135958653 | 12089 | 2: 136042952 | 12096 | 2: 136122518 |
| 12097 | 2: 136218516 | 12103 | 3: 192288 | 12172 | 3: 1379812 | 12218 | 3: 2245775 | 12219 | 3: 2335779 |
| 12428 | 3: 6422650 | 12571 | 3: 8746180 | 12573 | 3: 8864644 | 12579 | 3: 9185006 | 12699 | 3: 10683176 |
| 12753 | 3: 11779967 | 12757 | 3: 11849178 | 12758 | 3: 11934240 | 12759 | 3: 11998748 | 12811 | 3: 13516552 |
| 12962 | 3: 16327244 | 13006 | 3: 17313416 | 13007 | 3: 17408532 | 13036 | 3: 18384479 | 13087 | 3: 19384406 |
| 13088 | 3: 19480721 | 13103 | 3: 19938418 | 13104 | 3: 19975811 | 13105 | 3: 20062027 | 13114 | 3: 20219541 |
| 13115 | 3: 20275444 | 13134 | 3: 20949426 | 13135 | 3: 21041525 | 13139 | 3: 21260248 | 13153 | 3: 21701072 |
| 13164 | 3: 21935930 | 13203 | 3: 22637339 | 13253 | 3: 23859633 | 13388 | 3: 26747082 | 13484 | 3: 28324475 |
| 13485 | 3: 28415348 | 13486 | 3: 28426219 | 13487 | 3: 28519784 | 13535 | 3: 29477079 | 14013 | 3: 38769939 |
| 14015 | 3: 38908661 | 14018 | 3: 39010474 | 14019 | 3: 39102130 | 14042 | 3: 39825859 | 14046 | 3: 39962964 |
| 14061 | 3: 40312602 | 14077 | 3: 40804040 | 14079 | 3: 40921947 | 14112 | 3: 41900576 | 14171 | 3: 43138350 |
| 14186 | 3: 43521434 | 14223 | 3: 44490132 | 14224 | 3: 44561235 | 14250 | 3: 45348826 | 14257 | 3: 45588541 |
| 14268 | 3: 45973111 | 14270 | 3: 46106986 | 14307 | 3: 47132786 | 14310 | 3: 47350830 | 14401 | 3: 49233756 |
| 14490 | 3: 50263076 | 14511 | 3: 50885592 | 14519 | 3: 51298193 | 14525 | 3: 51591043 | 14548 | 3: 52087305 |
| 14576 | 3: 52675804 | 14622 | 3: 53900489 | 14624 | 3: 54093636 | 14635 | 3: 54224439 | 14636 | 3: 54256413 |
| 14637 | 3: 54344299 | 14639 | 3: 54400470 | 14640 | 3: 54475982 | 14641 | 3: 54564882 | 14642 | 3: 54618300 |
| 14643 | 3: 54697466 | 14644 | 3: 54793255 | 14645 | 3: 54888122 | 14679 | 3: 55617345 | 14680 | 3: 55712074 |
| 14682 | 3: 55757845 | 14683 | 3: 55857361 | 14699 | 3: 56411615 | 14703 | 3: 56614407 | 14724 | 3: 57120778 |
| 14725 | 3: 57210410 | 14730 | 3: 57316623 | 14763 | 3: 58252296 | 14807 | 3: 59522050 | 14820 | 3: 59954303 |
| 14878 | 3: 61139535 | 14937 | 3: 62995597 | 14986 | 3: 64406567 | 15080 | 3: 66682938 | 15081 | 3: 66782644 |
| 15094 | 3: 67125573 | 15179 | 3: 69231454 | 15232 | 3: 70770801 | 15263 | 3: 72024952 | 15274 | 3: 72399723 |
| 15277 | 3: 72561296 | 15278 | 3: 72658767 | 15282 | 3: 72860663 | 15323 | 3: 74163128 | 15334 | 3: 74460656 |
| 15335 | 3: 74560466 | 15373 | 3: 75613962 | 15388 | 3: 75959257 | 15396 | 3: 76253283 | 15407 | 3: 76546947 |
| 15518 | 3: 79853923 | 15527 | 3: 80073268 | 15551 | 3: 80845482 | 15603 | 3: 82127843 | 15647 | 3: 83276598 |
| 15687 | 3: 84374682 | 15749 | 3: 85174617 | 15864 | 3: 87868064 | 16131 | 3: 93389797 | 16146 | 3: 93816462 |
| 16173 | 3: 94470905 | 16174 | 3: 94570511 | 16205 | 3: 95542077 | 16234 | 3: 96375723 | 16236 | 3: 96516771 |
| 16319 | 3: 98506422 | 16487 | 3: 101953130 | 16551 | 3: 103287865 | 17051 | 3: 112888751 | 17107 | 3: 113766186 |
| 17462 | 3: 119142510 | 17463 | 3: 119241254 | 17465 | 3: 119312183 | 17466 | 3: 119337850 | 17471 | 3: 119559889 |
| 17490 | 3: 120011571 | 17505 | 3: 120402369 | 17508 | 3: 120529061 | 17527 | 4: 196536 | 17528 | 4: 280153 |
| 17533 | 4: 535625 | 17539 | 4: 791948 | 17549 | 4: 1260383 | 17552 | 4: 1417558 | 17600 | 4: 2614737 |
| 17745 | 4: 5912982 | 17995 | 4: 11174189 | 18042 | 4: 12473770 | 18087 | 4: 13462303 | 18127 | 4: 14704975 |
| 18131 | 4: 14787436 | 18168 | 4: 15690889 | 18178 | 4: 16124536 | 18250 | 4: 17859359 | 18322 | 4: 19353388 |
| 18383 | 4: 20445754 | 18433 | 4: 21454127 | 18434 | 4: 21541657 | 18445 | 4: 21872378 | 18462 | 4: 22281058 |
| 18501 | 4: 23239526 | 18518 | 4: 23775334 | 18529 | 4: 24057792 | 18561 | 4: 25063606 | 18581 | 4: 25549415 |
| 18709 | 4: 28892826 | 18713 | 4: 29073099 | 18729 | 4: 29642872 | 18740 | 4: 30014475 | 18741 | 4: 30101996 |
| 18846 | 4: 32916549 | 18904 | 4: 34496286 | 18926 | 4: 35328524 | 19007 | 4: 37643101 | 19054 | 4: 38955171 |
| 19106 | 4: 40318271 | 19156 | 4: 41249802 | 19160 | 4: 41529195 | 19161 | 4: 41619769 | 19165 | 4: 41769265 |
| 19169 | 4: 41869434 | 19170 | 4: 41950512 | 19184 | 4: 42417863 | 19237 | 4: 43892488 | 19281 | 4: 45005168 |
| 19322 | 4: 46242543 | 19325 | 4: 46409911 | 19326 | 4: 46490707 | 19407 | 4: 48377180 | 19408 | 4: 48467232 |
| 19487 | 4: 50087185 | 19488 | 4: 50186156 | 19499 | 4: 50633232 | 19510 | 4: 50996653 | 19537 | 4: 51864991 |
| 19612 | 4: 53951273 | 19613 | 4: 54035561 | 19616 | 4: 54191462 | 19618 | 4: 54332820 | 19619 | 4: 54391051 |
| 19629 | 4: 54743084 | 19640 | 4: 55160033 | 19642 | 4: 55344926 | 19687 | 4: 56506421 | 19766 | 4: 59069599 |
| 19793 | 4: 59746110 | 19805 | 4: 60065778 | 19828 | 4: 60746730 | 19861 | 4: 61706369 | 19892 | 4: 62405503 |
| 19897 | 4: 62630427 | 19898 | 4: 62713783 | 19987 | 4: 65201660 | 19989 | 4: 65245742 | 19990 | 4: 65343359 |
| 19991 | 4: 65436101 | 19993 | 4: 65541264 | 20071 | 4: 67301224 | 20088 | 4: 67785324 | 20101 | 4: 68075914 |
| 20135 | 4: 69146493 | 20160 | 4: 69711048 | 20161 | 4: 69810925 | 20204 | 4: 70819803 | 20231 | 4: 71683652 |
| 20252 | 4: 72269219 | 20253 | 4: 72368269 | 20301 | 4: 73885846 | 20309 | 4: 74101412 | 20450 | 4: 77371853 |
| 20475 | 4: 77790836 | 20529 | 4: 79467619 | 20647 | 4: 82182401 | 20730 | 4: 84549829 | 20733 | 4: 84649340 |
| 20835 | 4: 87133726 | 20859 | 4: 87827889 | 20910 | 4: 89303585 | 20944 | 4: 90250683 | 20961 | 4: 90701139 |
| 20978 | 4: 91069757 | 21084 | 4: 93203600 | 21136 | 4: 94310305 | 21160 | 4: 95074007 | 21254 | 4: 96620212 |
| 21255 | 4: 96718455 | 21485 | 4: 99029738 | 21489 | 4: 99310528 | 21491 | 4: 99423168 | 21610 | 4: 101894551 |
| 21616 | 4: 102178027 | 21696 | 4: 104152919 | 21762 | 4: 105592184 | 21763 | 4: 105606470 | 21764 | 4: 105706248 |
| 21771 | 4: 105878830 | 21772 | 4: 105960151 | 21945 | 4: 109512073 | 22167 | 4: 113058361 | 22168 | 4: 113156968 |
| 22546 | 4: 119885612 | 22565 | 5: 195803 | 22566 | 5: 239346 | 22567 | 5: 334945 | 22569 | 5: 484209 |
| 22570 | 5: 577221 | 22620 | 5: 1547817 | 22706 | 5: 3643809 | 22817 | 5: 6128913 | 22818 | 5: 6209013 |
| 23050 | 5: 11616643 | 23133 | 5: 13446994 | 23146 | 5: 13786322 | 23170 | 5: 14539492 | 23173 | 5: 14638005 |
| 23183 | 5: 14893917 | 23248 | 5: 16007217 | 23258 | 5: 16431067 | 23300 | 5: 17571373 | 23303 | 5: 17775869 |
| 23312 | 5: 18094567 | 23332 | 5: 18790466 | 23643 | 5: 26034515 | 23644 | 5: 26061374 | 23645 | 5: 26151322 |
| 23657 | 5: 26429773 | 23772 | 5: 28790811 | 23781 | 5: 29074134 | 23866 | 5: 30986242 | 23867 | 5: 31079915 |
| 23893 | 5: 31837129 | 23983 | 5: 34087072 | 23986 | 5: 34286241 | 23987 | 5: 34305687 | 23988 | 5: 34375513 |
| 23989 | 5: 34474025 | 23993 | 5: 34687154 | 24020 | 5: 35185968 | 24025 | 5: 35351867 | 24034 | 5: 35659101 |
| 24055 | 5: 36320021 | 24070 | 5: 36698940 | 24073 | 5: 36843791 | 24100 | 5: 37690831 | 24156 | 5: 38824022 |
| 24222 | 5: 40535193 | 24224 | 5: 40667896 | 24269 | 5: 42106898 | 24276 | 5: 42475432 | 24298 | 5: 42980132 |
| 24308 | 5: 43193547 | 24330 | 5: 43788410 | 24355 | 5: 44185303 | 24356 | 5: 44283317 | 24411 | 5: 45870645 |
| 24496 | 5: 47715438 | 24508 | 5: 48068765 | 24588 | 5: 50328496 | 24634 | 5: 51301107 | 24681 | 5: 52618260 |
| 24684 | 5: 52742025 | 24687 | 5: 52972061 | 24709 | 5: 53722470 | 24719 | 5: 53987712 | 24800 | 5: 55749160 |
| 24856 | 5: 57057069 | 24875 | 5: 57516539 | 24885 | 5: 57746077 | 24886 | 5: 57832714 | 24887 | 5: 57930196 |
| 24906 | 5: 58383434 | 24909 | 5: 58493473 | 24913 | 5: 58628294 | 24914 | 5: 58693980 | 24917 | 5: 58889899 |
| 24918 | 5: 58986587 | 24932 | 5: 59477581 | 24935 | 5: 59606022 | 24937 | 5: 59692009 | 24938 | 5: 59786270 |
| 24966 | 5: 60382341 | 24967 | 5: 60450259 | 25017 | 5: 61851862 | 25115 | 5: 64573229 | 25212 | 5: 67004367 |
| 25214 | 5: 67099597 | 25215 | 5: 67183935 | 25245 | 5: 67610309 | 25287 | 5: 68696282 | 25352 | 5: 70049824 |
| 25368 | 5: 70612128 | 25480 | 5: 72974385 | 25499 | 5: 73530687 | 25534 | 5: 74251346 | 25535 | 5: 74343381 |
| 25593 | 5: 75456468 | 25594 | 5: 75473431 | 25595 | 5: 75561363 | 25668 | 5: 77473876 | 25669 | 5: 77528763 |
| 25676 | 5: 77903820 | 25690 | 5: 78146870 | 25691 | 5: 78242136 | 25730 | 5: 79116954 | 25737 | 5: 79370071 |
| 25738 | 5: 79464943 | 25739 | 5: 79516884 | 25762 | 5: 80354597 | 25763 | 5: 80452900 | 25786 | 5: 81078405 |
| 25790 | 5: 81259940 | 25866 | 5: 82675565 | 25872 | 5: 82889086 | 26084 | 5: 84839297 | 26119 | 5: 85681634 |
| 26136 | 5: 86133720 | 26140 | 5: 86239067 | 26194 | 5: 87412820 | 26322 | 5: 89485893 | 26392 | 5: 91260025 |
| 26475 | 5: 92686818 | 26708 | 5: 97550215 | 26787 | 5: 99116271 | 26788 | 5: 99137116 | 26789 | 5: 99143867 |
| 26790 | 5: 99243441 | 26791 | 5: 99314005 | 26931 | 5: 102029541 | 26933 | 5: 102150740 | 26938 | 5: 102366923 |
| 26949 | 5: 102638331 | 26950 | 5: 102641378 | 26951 | 5: 102662950 | 26952 | 5: 102812214 | 26953 | 5: 102878588 |
| 26954 | 5: 102970619 | 26955 | 5: 103010571 | 27120 | 5: 106009983 | 27279 | 5: 108384166 | 27298 | 5: 108674540 |
| 27332 | 5: 109217271 | 27333 | 5: 109314750 | 27544 | 5: 113514180 | 27545 | 5: 113554598 | 27575 | 5: 114440055 |
| 27576 | 5: 114536143 | 27578 | 5: 114640058 | 27595 | 5: 115151924 | 27738 | 5: 116870418 | 27938 | 6: 97487 |
| 27946 | 6: 394469 | 27947 | 6: 492748 | 27979 | 6: 1193084 | 28037 | 6: 2239422 | 28060 | 6: 2768563 |
| 28130 | 6: 3837662 | 28184 | 6: 5212458 | 28195 | 6: 5517626 | 28196 | 6: 5615037 | 28314 | 6: 8143447 |
| 28328 | 6: 8615351 | 28329 | 6: 8684262 | 28437 | 6: 11262662 | 28551 | 6: 13520510 | 28553 | 6: 13697509 |
| 28571 | 6: 14245516 | 28744 | 6: 17861677 | 28918 | 6: 21562233 | 29086 | 6: 25221507 | 29099 | 6: 25661883 |
| 29133 | 6: 26462640 | 29181 | 6: 27717652 | 29384 | 6: 31231051 | 29419 | 6: 32134214 | 29424 | 6: 32438836 |
| 29434 | 6: 32698791 | 29437 | 6: 32783759 | 29438 | 6: 32866982 | 32354 | 6: 44532925 | 32523 | 6: 45804379 |
| 32559 | 6: 46543851 | 32571 | 6: 46849949 | 32625 | 6: 48139435 | 32639 | 6: 48619247 | 32722 | 6: 50593610 |
| 32763 | 6: 51940574 | 32772 | 6: 52186933 | 32883 | 6: 54324568 | 32961 | 6: 55908779 | 32979 | 6: 56522044 |
| 33028 | 6: 57631443 | 33029 | 6: 57728837 | 33054 | 6: 58492711 | 33067 | 6: 58822636 | 33096 | 6: 59668604 |
| 33161 | 6: 61063375 | 33203 | 6: 62168026 | 33214 | 6: 62509742 | 33219 | 6: 62716293 | 33266 | 6: 64072884 |
| 33270 | 6: 64250794 | 33339 | 6: 66605824 | 33359 | 6: 67153345 | 33441 | 6: 69518831 | 33443 | 6: 69655680 |
| 33453 | 6: 69965893 | 33454 | 6: 70058818 | 33456 | 6: 70166659 | 33473 | 6: 70643646 | 33511 | 6: 71802143 |
| 33591 | 6: 74410889 | 33596 | 6: 74629845 | 33599 | 6: 74741424 | 33617 | 6: 75350029 | 33619 | 6: 75446552 |
| 33621 | 6: 75621675 | 33636 | 6: 75990894 | 33639 | 6: 76209600 | 33640 | 6: 76275919 | 33643 | 6: 76450277 |
| 33706 | 6: 78238972 | 33708 | 6: 78363832 | 33713 | 6: 78527838 | 33724 | 6: 79038243 | 33745 | 6: 79831508 |
| 33795 | 6: 81332996 | 33807 | 6: 81870711 | 33808 | 6: 81964290 | 33862 | 6: 83422593 | 33883 | 6: 83749295 |
| 33906 | 6: 84274386 | 33923 | 6: 84775788 | 33989 | 6: 86113898 | 34365 | 6: 93730394 | 34406 | 6: 94508641 |
| 34407 | 6: 94604426 | 34408 | 6: 94671907 | 34418 | 6: 95161943 | 34422 | 6: 95280868 | 34621 | 6: 99278886 |
| 34645 | 6: 100084140 | 34895 | 6: 104783662 | 34926 | 6: 105376492 | 35015 | 6: 107674685 | 35016 | 6: 107769675 |
| 35048 | 6: 108791274 | 35098 | 6: 109783321 | 35256 | 6: 113049419 | 35257 | 6: 113144284 | 35385 | 6: 115461946 |
| 35450 | 6: 116698963 | 35451 | 6: 116752334 | 35452 | 6: 116848436 | 35454 | 6: 117023687 | 35455 | 6: 117121869 |
| 35460 | 6: 117289738 | 35461 | 6: 117373173 | 35468 | 6: 117512770 | 35471 | 6: 117661215 | 35472 | 6: 117698401 |
| 35473 | 6: 117792640 | 35474 | 7: 80741 | 35475 | 7: 164546 | 35720 | 7: 4691508 | 35922 | 7: 7350133 |
| 35945 | 7: 8005827 | 35954 | 7: 8250300 | 35957 | 7: 8423976 | 35962 | 7: 8644832 | 35963 | 7: 8715092 |
| 35964 | 7: 8798532 | 35965 | 7: 8829774 | 35967 | 7: 9006597 | 35968 | 7: 9034045 | 35969 | 7: 9111685 |
| 35970 | 7: 9205519 | 35971 | 7: 9294350 | 35972 | 7: 9323285 | 35973 | 7: 9420386 | 35974 | 7: 9427375 |
| 35975 | 7: 9522686 | 35976 | 7: 9608447 | 35980 | 7: 9782711 | 35981 | 7: 9853483 | 35982 | 7: 9893322 |
| 35983 | 7: 9935560 | 35987 | 7: 10196676 | 35988 | 7: 10292183 | 35993 | 7: 10432630 | 35994 | 7: 10494698 |
| 35995 | 7: 10588854 | 36000 | 7: 10694692 | 36001 | 7: 10709248 | 36002 | 7: 10799397 | 36003 | 7: 10898948 |
| 36004 | 7: 10972220 | 36034 | 7: 11621411 | 36038 | 7: 11733951 | 36290 | 7: 15957756 | 36351 | 7: 17374656 |
| 36373 | 7: 17800196 | 36382 | 7: 18107639 | 36530 | 7: 20567856 | 36589 | 7: 21886187 | 36612 | 7: 22364050 |
| 36643 | 7: 23141409 | 36693 | 7: 24266298 | 36997 | 7: 31102265 | 37168 | 7: 34931021 | 37197 | 7: 35685794 |
| 37213 | 7: 36102653 | 37218 | 7: 36242365 | 37331 | 7: 38727405 | 37345 | 7: 39057017 | 37432 | 7: 40812159 |
| 37449 | 7: 41102749 | 37450 | 7: 41174068 | 37456 | 7: 41391176 | 37460 | 7: 41530820 | 37465 | 7: 41621787 |
| 37485 | 7: 42209152 | 37489 | 7: 42394156 | 37545 | 7: 43685836 | 37553 | 7: 43941722 | 37559 | 7: 44261005 |
| 37564 | 7: 44505025 | 37616 | 7: 45789210 | 37618 | 7: 45899026 | 37622 | 7: 46066923 | 37702 | 7: 47884765 |
| 37704 | 7: 47992422 | 37706 | 7: 48152895 | 37757 | 7: 49264671 | 37763 | 7: 49508927 | 37764 | 7: 49551981 |
| 37765 | 7: 49632647 | 37766 | 7: 49732360 | 37767 | 7: 49825415 | 37772 | 7: 50083648 | 37773 | 7: 50141438 |
| 37774 | 7: 50238296 | 37775 | 7: 50295962 | 37776 | 7: 50392227 | 37778 | 7: 50523822 | 37782 | 7: 50734140 |
| 37785 | 7: 50857210 | 37786 | 7: 50956025 | 37795 | 7: 51298758 | 37805 | 7: 51627529 | 37808 | 7: 51770556 |
| 37810 | 7: 51904626 | 37877 | 7: 52516644 | 37913 | 7: 53384544 | 37926 | 7: 53682528 | 37936 | 7: 54073527 |
| 38031 | 7: 56472724 | 38061 | 7: 57477755 | 38086 | 7: 58105186 | 38087 | 7: 58204392 | 38167 | 7: 59670910 |
| 38195 | 7: 60290921 | 38291 | 7: 62241248 | 38363 | 7: 63608853 | 38381 | 7: 64051258 | 38502 | 7: 66283774 |
| 38508 | 7: 66535109 | 38570 | 7: 68229559 | 38620 | 7: 69315564 | 38662 | 7: 70240253 | 38678 | 7: 70856473 |
| 38773 | 7: 73340932 | 38774 | 7: 73425725 | 38792 | 7: 73921199 | 38812 | 7: 74461479 | 39002 | 7: 78855864 |
| 39182 | 7: 82082096 | 39313 | 7: 85461499 | 39332 | 7: 86158728 | 39339 | 7: 86335874 | 39344 | 7: 86445479 |
| 39366 | 7: 86982142 | 39476 | 7: 89945029 | 39477 | 7: 90039805 | 39669 | 7: 93367867 | 39716 | 7: 94784594 |
| 40029 | 7: 98642157 | 40031 | 7: 98712459 | 40032 | 7: 98793113 | 40136 | 7: 101498466 | 40233 | 7: 103750242 |
| 40234 | 7: 103835986 | 40277 | 7: 105099277 | 40483 | 7: 110118836 | 40484 | 7: 110198554 | 40485 | 7: 110298545 |
| 40486 | 7: 110364995 | 40487 | 7: 110462011 | 40488 | 7: 110524775 | 40489 | 7: 110616075 | 40491 | 8: 119077 |
| 40492 | 8: 204055 | 40502 | 8: 510254 | 40549 | 8: 1447144 | 40550 | 8: 1539600 | 40593 | 8: 2298980 |
| 40618 | 8: 3109616 | 40678 | 8: 4419528 | 40679 | 8: 4488605 | 40713 | 8: 5356367 | 40792 | 8: 7353398 |
| 40816 | 8: 7715961 | 40887 | 8: 9573171 | 40913 | 8: 10172186 | 41018 | 8: 12543611 | 41019 | 8: 12642666 |
| 41081 | 8: 14746899 | 41091 | 8: 15001201 | 41101 | 8: 15260009 | 41198 | 8: 17406666 | 41267 | 8: 19472727 |
| 41298 | 8: 20253347 | 41380 | 8: 22549276 | 41383 | 8: 22747845 | 41388 | 8: 22913643 | 41395 | 8: 23313119 |
| 41446 | 8: 24571671 | 41627 | 8: 27179633 | 41661 | 8: 28342382 | 41677 | 8: 28819278 | 41709 | 8: 29645861 |
| 41763 | 8: 30913973 | 41814 | 8: 32226362 | 41825 | 8: 32643213 | 41830 | 8: 32879092 | 41831 | 8: 32974702 |
| 41833 | 8: 33127475 | 41844 | 8: 33407993 | 41845 | 8: 33470771 | 41900 | 8: 34950717 | 41905 | 8: 35118252 |
| 41930 | 8: 35658609 | 42020 | 8: 37747312 | 42042 | 8: 38560565 | 42053 | 8: 38837520 | 42054 | 8: 38936996 |
| 42058 | 8: 39131324 | 42203 | 8: 42579916 | 42214 | 8: 42818923 | 42264 | 8: 44335672 | 42287 | 8: 45011011 |
| 42294 | 8: 45233429 | 42414 | 8: 47856381 | 42435 | 8: 48611381 | 42541 | 8: 51528439 | 42598 | 8: 52866048 |
| 42705 | 8: 55793930 | 42720 | 8: 56175135 | 42793 | 8: 57947632 | 42804 | 8: 58378680 | 42806 | 8: 58511840 |
| 42900 | 8: 60710884 | 42937 | 8: 61860162 | 42990 | 8: 63095889 | 42996 | 8: 63293941 | 42997 | 8: 63391647 |
| 43046 | 8: 64688332 | 43070 | 8: 65351408 | 43083 | 8: 65593070 | 43086 | 8: 65766665 | 43108 | 8: 66276105 |
| 43137 | 8: 66960690 | 43199 | 8: 68392696 | 43246 | 8: 69535824 | 43279 | 8: 70389972 | 43369 | 8: 72702268 |
| 43466 | 8: 75072947 | 43494 | 8: 76094638 | 43555 | 8: 77086614 | 43558 | 8: 77243628 | 43569 | 8: 77734663 |
| 43571 | 8: 77844702 | 43601 | 8: 78788179 | 43610 | 8: 79140878 | 43615 | 8: 79417890 | 43732 | 8: 82254632 |
| 43780 | 8: 83455155 | 43784 | 8: 83630961 | 43864 | 8: 85507118 | 43904 | 8: 86456661 | 43907 | 8: 86622904 |
| 43962 | 8: 87959678 | 43976 | 8: 88341264 | 44029 | 8: 89430089 | 44104 | 8: 91188437 | 44105 | 8: 91284358 |
| 44119 | 8: 91608918 | 44148 | 8: 92091641 | 44201 | 8: 93754674 | 44213 | 8: 94306363 | 44214 | 8: 94402192 |
| 44215 | 8: 94469316 | 44280 | 8: 96346983 | 44433 | 8: 99864196 | 44481 | 8: 101198784 | 44501 | 8: 101698974 |
| 44683 | 8: 104921232 | 44740 | 8: 106178029 | 44742 | 8: 106321176 | 44817 | 8: 108369912 | 44818 | 8: 108466305 |
| 44826 | 8: 108830921 | 44827 | 8: 108930028 | 44840 | 8: 109399618 | 44844 | 8: 109663372 | 44876 | 8: 110607864 |
| 44902 | 8: 111508670 | 44903 | 8: 111524854 | 44904 | 8: 111672008 | 44905 | 8: 111767142 | 44906 | 8: 111818202 |
| 44907 | 8: 111891683 | 44908 | 8: 111919894 | 44909 | 8: 112058818 | 44910 | 8: 112150362 | 44911 | 8: 112232760 |
| 44912 | 8: 112294473 | 44913 | 8: 112461632 | 44914 | 8: 112537285 | 44915 | 8: 112604404 | 44916 | 8: 112658175 |
| 44917 | 8: 112746559 | 44918 | 8: 112818233 | 44919 | 8: 112865068 | 44920 | 8: 112932415 | 44921 | 8: 112969679 |
| 44922 | 8: 112981176 | 44923 | 8: 113097953 | 44924 | 8: 113163827 | 44925 | 8: 113180320 | 44926 | 8: 113252524 |
| 44930 | 9: 190038 | 44931 | 9: 289864 | 45031 | 9: 2539725 | 45074 | 9: 3743248 | 45089 | 9: 4213848 |
| 45112 | 9: 4988015 | 45135 | 9: 5683174 | 45179 | 9: 7188401 | 45252 | 9: 8906729 | 45274 | 9: 9555271 |
| 45378 | 9: 10911653 | 45502 | 9: 13481091 | 45518 | 9: 13965555 | 45582 | 9: 15291327 | 45748 | 9: 19358233 |
| 45798 | 9: 20933619 | 45807 | 9: 21235916 | 45808 | 9: 21329366 | 46023 | 9: 26340073 | 46029 | 9: 26560747 |
| 46194 | 9: 29938464 | 46280 | 9: 31821673 | 46371 | 9: 34153811 | 46384 | 9: 34492853 | 46503 | 9: 37375637 |
| 46515 | 9: 37681606 | 46604 | 9: 39416078 | 46605 | 9: 39496518 | 46691 | 9: 41269453 | 46692 | 9: 41353434 |
| 46707 | 9: 41741270 | 46708 | 9: 41834289 | 46710 | 9: 41949048 | 46773 | 9: 43557020 | 46780 | 9: 43780420 |
| 46834 | 9: 45079509 | 46835 | 9: 45168173 | 46839 | 9: 45353183 | 46856 | 9: 45976363 | 46867 | 9: 46356308 |
| 46879 | 9: 46772812 | 46892 | 9: 47186026 | 46935 | 9: 48538260 | 47033 | 9: 51244082 | 47100 | 9: 53108085 |
| 47125 | 9: 53577095 | 47177 | 9: 55074875 | 47178 | 9: 55120285 | 47179 | 9: 55219943 | 47205 | 9: 56052958 |
| 47226 | 9: 56606356 | 47254 | 9: 57407794 | 47256 | 9: 57496462 | 47281 | 9: 58031623 | 47311 | 9: 59018652 |
| 47408 | 9: 61120108 | 47411 | 9: 61356232 | 47442 | 9: 62281541 | 47443 | 9: 62376661 | 47493 | 9: 63781683 |
| 47496 | 9: 63979099 | 47499 | 9: 64121913 | 47535 | 9: 64946776 | 47643 | 9: 66726405 | 47644 | 9: 66806876 |
| 47646 | 9: 66932759 | 47655 | 9: 67186941 | 47715 | 9: 68688996 | 47850 | 9: 71852129 | 47857 | 9: 72065985 |
| 47858 | 9: 72161733 | 47894 | 9: 73058318 | 47968 | 9: 74896771 | 48054 | 9: 77165874 | 48179 | 9: 79135168 |
| 48188 | 9: 79446217 | 48426 | 9: 84561403 | 48431 | 9: 84647624 | 48432 | 9: 84745238 | 48521 | 9: 86545282 |
| 48523 | 9: 86670733 | 48544 | 9: 87047352 | 48545 | 9: 87102465 | 48546 | 9: 87196437 | 48547 | 9: 87295910 |
| 48615 | 9: 88318893 | 48799 | 9: 91914972 | 48879 | 9: 93747256 | 49154 | 9: 98379141 | 49230 | 9: 99867971 |
| 49483 | 9: 104102942 | 49484 | 9: 104107451 | 49486 | 9: 104142058 | 49488 | 9: 104277105 | 49495 | 9: 104453514 |
| 49497 | 9: 104550915 | 49498 | 9: 104641226 | 49499 | 9: 104641308 | 49500 | 9: 104641495 | 49502 | 10: 184460 |
| 49503 | 10: 213217 | 49504 | 10: 295234 | 49506 | 10: 344867 | 49507 | 10: 435671 | 49566 | 10: 1764102 |
| 49836 | 10: 6862089 | 49850 | 10: 7338516 | 49919 | 10: 8633030 | 49991 | 10: 10372137 | 50045 | 10: 11267448 |
| 50115 | 10: 12952255 | 50434 | 10: 18833922 | 50435 | 10: 18841050 | 50436 | 10: 18845495 | 50437 | 10: 18848616 |
| 50438 | 10: 18854031 | 50469 | 10: 19429779 | 50470 | 10: 19516186 | 50638 | 10: 22676353 | 50639 | 10: 22775635 |
| 50642 | 10: 22836164 | 50643 | 10: 22909428 | 50646 | 10: 23059842 | 50647 | 10: 23066056 | 50648 | 10: 23228975 |
| 50649 | 10: 23268766 | 50650 | 10: 23368614 | 50652 | 10: 23464004 | 50653 | 10: 23533419 | 50654 | 10: 23627755 |
| 50655 | 10: 23671760 | 50656 | 10: 23769788 | 50657 | 10: 23771464 | 50658 | 10: 24790738 | 50659 | 10: 24799735 |
| 50660 | 10: 24818929 | 50661 | 10: 24913820 | 50662 | 10: 24923736 | 50663 | 10: 25022602 | 50664 | 10: 25057985 |
| 50665 | 10: 25156372 | 50666 | 10: 25164147 | 50667 | 10: 25241806 | 50668 | 10: 25341513 | 50680 | 10: 25758684 |
| 50778 | 10: 27556986 | 50900 | 10: 29890120 | 50926 | 10: 30807403 | 51080 | 10: 34810626 | 51145 | 10: 36132543 |
| 51146 | 10: 36213908 | 51160 | 10: 36596113 | 51163 | 10: 36787409 | 51240 | 10: 38618727 | 51259 | 10: 39073733 |
| 51260 | 10: 39169136 | 51284 | 10: 39715696 | 51292 | 10: 39993149 | 51302 | 10: 40306626 | 51390 | 10: 42652463 |
| 51403 | 10: 43115839 | 51417 | 10: 43643966 | 51433 | 10: 44096569 | 51474 | 10: 45124642 | 51490 | 10: 45511746 |
| 51745 | 10: 50796271 | 51861 | 10: 53660012 | 51942 | 10: 56043883 | 51992 | 10: 57210937 | 52035 | 10: 58319989 |
| 52040 | 10: 58481438 | 52054 | 10: 58874663 | 52071 | 10: 59336872 | 52092 | 10: 60028136 | 52123 | 10: 61231374 |
| 52126 | 10: 61443371 | 52158 | 10: 62042620 | 52193 | 10: 62708109 | 52228 | 10: 63495064 | 52236 | 10: 63804854 |
| 52247 | 10: 64189747 | 52294 | 10: 65251624 | 52312 | 10: 65561116 | 52344 | 10: 66259714 | 52392 | 10: 67327408 |
| 52523 | 10: 70522773 | 52524 | 10: 70601203 | 52526 | 10: 70690873 | 52527 | 10: 70781098 | 52548 | 10: 71377715 |
| 52590 | 10: 72631813 | 52591 | 10: 72727860 | 52596 | 10: 72983865 | 52597 | 10: 73076966 | 52643 | 10: 74135935 |
| 52664 | 10: 74941316 | 52700 | 10: 75976569 | 52736 | 10: 76707628 | 53015 | 10: 82572013 | 53034 | 10: 83144860 |
| 53169 | 10: 86010466 | 53423 | 10: 91110721 | 53454 | 10: 92010714 | 53519 | 10: 93279984 | 53520 | 10: 93370297 |
| 53640 | 10: 96340328 | 53705 | 10: 97729030 | 53779 | 10: 99321961 | 53802 | 10: 99841614 | 53981 | 10: 10327740 |
| 54006 | 11: 514378 | 54266 | 11: 4322857 | 54313 | 11: 5395664 | 54364 | 11: 6596735 | 54435 | 11: 8259962 |
| 54438 | 11: 8462700 | 54446 | 11: 8761089 | 54705 | 11: 14474472 | 54822 | 11: 17458053 | 54838 | 11: 17908654 |
| 54839 | 11: 17983413 | 54843 | 11: 18162535 | 54847 | 11: 18333639 | 54889 | 11: 19456930 | 54900 | 11: 19732194 |
| 54920 | 11: 20390071 | 54993 | 11: 22091427 | 55041 | 11: 23355951 | 55079 | 11: 24188498 | 55126 | 11: 25171864 |
| 55148 | 11: 25792655 | 55180 | 11: 26465214 | 55181 | 11: 26466574 | 55182 | 11: 26531940 | 55361 | 11: 30575172 |
| 55422 | 11: 32139468 | 55488 | 11: 33907492 | 55506 | 11: 34748337 | 55580 | 11: 36801995 | 55638 | 11: 37982330 |
| 55648 | 11: 38441743 | 55686 | 11: 39673530 | 55712 | 11: 40436923 | 55715 | 11: 40647883 | 55794 | 11: 42338919 |
| 55853 | 11: 43645607 | 55866 | 11: 44133002 | 55918 | 11: 45401293 | 55920 | 11: 45518720 | 55922 | 11: 45703344 |
| 55923 | 11: 45779776 | 55956 | 11: 46344663 | 56060 | 11: 48391618 | 56182 | 11: 50484454 | 56211 | 11: 51437117 |
| 56233 | 11: 52229998 | 56236 | 11: 52492485 | 56254 | 11: 53121751 | 56260 | 11: 53366816 | 56270 | 11: 53745957 |
| 56342 | 11: 55279305 | 56373 | 11: 55996334 | 56379 | 11: 56241704 | 56381 | 11: 56406566 | 56386 | 11: 56550578 |
| 56393 | 11: 56780643 | 56413 | 11: 57282162 | 56423 | 11: 57597675 | 56424 | 11: 57696611 | 56449 | 11: 58487002 |
| 56502 | 11: 59891961 | 56507 | 11: 60131924 | 56508 | 11: 60226569 | 56583 | 11: 61202324 | 56584 | 11: 61289988 |
| 56586 | 11: 61410046 | 56587 | 11: 61495664 | 56592 | 11: 61644339 | 56593 | 11: 61726332 | 56717 | 11: 64661124 |
| 56765 | 11: 65925707 | 56800 | 11: 66728012 | 56888 | 11: 68240104 | 56900 | 11: 68629264 | 56946 | 11: 69472443 |
| 57044 | 11: 70953054 | 57115 | 11: 72333655 | 57123 | 11: 72679475 | 57165 | 11: 73412263 | 57169 | 11: 73601533 |
| 57220 | 11: 74558447 | 57221 | 11: 74602747 | 57223 | 11: 74706334 | 57268 | 11: 75776010 | 57283 | 11: 76197120 |
| 57467 | 11: 79506772 | 57675 | 11: 84372612 | 57684 | 11: 84807135 | 57765 | 11: 86512168 | 57810 | 11: 87538574 |
| 57811 | 11: 87624731 | 57982 | 11: 90885707 | 58110 | 11: 93370799 | 58146 | 11: 94432248 | 58203 | 11: 95957372 |
| 58367 | 11: 99599606 | 58410 | 11: 10044124 | 58478 | 11: 10165527 | 58566 | 11: 10321963 | 58606 | 11: 10400943 |
| 58677 | 11: 10536644 | 58680 | 11: 10545702 | 58708 | 11: 10585665 | 58709 | 11: 10595532 | 58714 | 11: 10618317 |
| 58716 | 11: 10629012 | 58718 | 11: 10643100 | 58721 | 11: 10656647 | 58728 | 11: 10671924 | 58730 | 11: 10687276 |
| 58732 | 12: 91462 | 58765 | 12: 1113931 | 58768 | 12: 1245380 | 58884 | 12: 3705269 | 58963 | 12: 5912555 |
| 58975 | 12: 6345821 | 58982 | 12: 6709902 | 58998 | 12: 7126322 | 58999 | 12: 7224038 | 59220 | 12: 12834462 |
| 59327 | 12: 15203798 | 59530 | 12: 19527022 | 59607 | 12: 21552423 | 59610 | 12: 21691586 | 59611 | 12: 21787162 |
| 59641 | 12: 22535408 | 59688 | 12: 23635155 | 59689 | 12: 23728488 | 59791 | 12: 25996134 | 59792 | 12: 26092741 |
| 59794 | 12: 26216578 | 59804 | 12: 26581859 | 59875 | 12: 28358077 | 59905 | 12: 29299176 | 59926 | 12: 29941712 |
| 59950 | 12: 30491646 | 60005 | 12: 31739889 | 60020 | 12: 32049680 | 60093 | 12: 33950251 | 60098 | 12: 34160176 |
| 60110 | 12: 34560071 | 60129 | 12: 35057478 | 60167 | 12: 35870799 | 60171 | 12: 36164862 | 60174 | 12: 36313336 |
| 60188 | 12: 36646069 | 60194 | 12: 36915655 | 60217 | 12: 37496437 | 60218 | 12: 37593149 | 60219 | 12: 37662796 |
| 60221 | 12: 37752580 | 60223 | 12: 37929010 | 60224 | 12: 37976297 | 60225 | 12: 38053259 | 60230 | 12: 38238813 |
| 60242 | 12: 38895814 | 60247 | 12: 39058185 | 60250 | 12: 39172935 | 60271 | 12: 39830361 | 60272 | 12: 39927124 |
| 60297 | 12: 40565431 | 60302 | 12: 40827682 | 60314 | 12: 41265419 | 60318 | 12: 41435539 | 60322 | 12: 41606679 |
| 60337 | 12: 42104689 | 60343 | 12: 42399827 | 60348 | 12: 42626305 | 60349 | 12: 42724793 | 60353 | 12: 42919011 |
| 60357 | 12: 43020517 | 60359 | 12: 43190819 | 60369 | 12: 43612489 | 60370 | 12: 43698277 | 60374 | 12: 43993681 |
| 60398 | 12: 44816702 | 60406 | 12: 45079833 | 60408 | 12: 45170993 | 60432 | 12: 45962853 | 60433 | 12: 46045130 |
| 60515 | 12: 48438301 | 60537 | 12: 48906417 | 60622 | 12: 50638774 | 60623 | 12: 50712724 | 60795 | 12: 54440430 |
| 60879 | 12: 56915140 | 60896 | 12: 57520936 | 60897 | 12: 57618510 | 60917 | 12: 58198920 | 60927 | 12: 58558724 |
| 60980 | 12: 60328402 | 60984 | 12: 60431728 | 60991 | 12: 60833351 | 60998 | 12: 61166007 | 61017 | 12: 61818463 |
| 61069 | 12: 63224791 | 61091 | 12: 63866391 | 61114 | 12: 64636179 | 61130 | 12: 65016774 | 61202 | 12: 66945480 |
| 61203 | 12: 67032666 | 61345 | 12: 69830668 | 61346 | 12: 70008764 | 61347 | 12: 70092121 | 61349 | 12: 70199557 |
| 61350 | 12: 70296346 | 61351 | 12: 70314066 | 61352 | 12: 70373860 | 61354 | 12: 70528567 | 61355 | 12: 70625414 |
| 61356 | 12: 70723529 | 61359 | 12: 70754288 | 61360 | 12: 70832160 | 61361 | 12: 70921442 | 61362 | 12: 71020218 |
| 61363 | 12: 71027334 | 61364 | 12: 71106543 | 61365 | 12: 71199759 | 61375 | 12: 71408795 | 61376 | 12: 71457224 |
| 61377 | 12: 71551210 | 61384 | 12: 71729441 | 61392 | 12: 71965489 | 61393 | 12: 72058978 | 61394 | 12: 72147420 |
| 61395 | 12: 72230980 | 61396 | 12: 72265152 | 61397 | 12: 72337871 | 61398 | 12: 72374872 | 61399 | 12: 72490055 |
| 61400 | 12: 72498314 | 61401 | 12: 72637442 | 61402 | 12: 72712776 | 61403 | 12: 72812633 | 61902 | 12: 83578230 |
| 62097 | 12: 87141742 | 62098 | 13: 299895 | 62105 | 13: 682778 | 62106 | 13: 745802 | 62125 | 13: 1154063 |
| 62224 | 13: 2881031 | 62225 | 13: 2975142 | 62280 | 13: 4395240 | 62283 | 13: 4531236 | 62431 | 13: 8202540 |
| 62466 | 13: 9078686 | 62545 | 13: 10762211 | 62546 | 13: 10778845 | 62548 | 13: 10867741 | 62554 | 13: 11145952 |
| 62555 | 13: 11222783 | 62556 | 13: 11320371 | 62557 | 13: 11410445 | 62570 | 13: 12019870 | 62634 | 13: 13812138 |
| 62650 | 13: 14311743 | 62692 | 13: 15465789 | 62712 | 13: 16020130 | 62799 | 13: 17568011 | 62801 | 13: 17588165 |
| 62835 | 13: 18353118 | 63019 | 13: 21321597 | 63087 | 13: 23067175 | 63093 | 13: 23318050 | 63105 | 13: 23791918 |
| 63109 | 13: 23900966 | 63124 | 13: 24343896 | 63189 | 13: 25699872 | 63323 | 13: 29049594 | 63392 | 13: 30294381 |
| 63393 | 13: 30377425 | 63476 | 13: 31944092 | 63477 | 13: 32039794 | 63555 | 13: 33385230 | 63576 | 13: 34100138 |
| 63577 | 13: 34193250 | 63578 | 13: 34248535 | 63581 | 13: 34427463 | 63706 | 13: 37210289 | 63805 | 13: 39259881 |
| 63838 | 13: 39814181 | 64020 | 13: 43484075 | 64302 | 13: 48427057 | 64315 | 13: 49071500 | 64316 | 13: 49168510 |
| 64345 | 13: 49747887 | 64346 | 13: 49844262 | 64355 | 13: 50115519 | 64357 | 13: 50180207 | 64358 | 13: 50268295 |
| 64372 | 13: 50739564 | 64374 | 13: 50832795 | 64381 | 13: 51125057 | 64401 | 13: 51523719 | 64402 | 13: 51622569 |
| 64500 | 13: 53510732 | 64501 | 13: 53607473 | 64507 | 13: 53822879 | 64519 | 13: 54442428 | 64570 | 13: 55504923 |
| 64652 | 13: 57623027 | 64676 | 13: 58277988 | 64741 | 13: 59446084 | 64816 | 13: 61419709 | 64828 | 13: 61686658 |
| 64836 | 13: 61962725 | 64928 | 13: 63467047 | 64929 | 13: 63559780 | 64949 | 13: 64081941 | 65039 | 13: 65929411 |
| 65137 | 13: 68477424 | 65180 | 13: 69587905 | 65181 | 13: 69666536 | 65541 | 13: 77055890 | 65542 | 13: 77153724 |
| 65879 | 14: 93414 | 65895 | 14: 609870 | 65904 | 14: 947222 | 66104 | 14: 4750888 | 66610 | 14: 13439017 |
| 66611 | 14: 13473101 | 66612 | 14: 13553001 | 66613 | 14: 13632606 | 66614 | 14: 13721229 | 66615 | 14: 13734707 |
| 66616 | 14: 13832328 | 66786 | 14: 17490496 | 66822 | 14: 18129262 | 66823 | 14: 18227619 | 66827 | 14: 18277849 |
| 66828 | 14: 18376690 | 66874 | 14: 19576484 | 67559 | 14: 25819002 | 67636 | 14: 26880974 | 67665 | 14: 27799331 |
| 67800 | 14: 30971186 | 67869 | 14: 32582584 | 67898 | 14: 33242218 | 67971 | 14: 34837449 | 67972 | 14: 34885142 |
| 67977 | 14: 35171625 | 68004 | 14: 35658219 | 68094 | 14: 38119848 | 68123 | 14: 38980550 | 68157 | 14: 40059476 |
| 68173 | 14: 40339877 | 68272 | 14: 43115419 | 68466 | 14: 47686908 | 68471 | 14: 47920443 | 68533 | 14: 49442868 |
| 68573 | 14: 50805531 | 68645 | 14: 52567142 | 68650 | 14: 52653514 | 68657 | 14: 52955563 | 68658 | 14: 53054564 |
| 68676 | 14: 53669390 | 68747 | 14: 54918170 | 68781 | 14: 55712044 | 68782 | 14: 55807243 | 68783 | 14: 55875273 |
| 68784 | 14: 55953452 | 68824 | 14: 57218378 | 68935 | 14: 59606995 | 68952 | 14: 60129379 | 68980 | 14: 60666003 |
| 68981 | 14: 60764297 | 69127 | 14: 63797690 | 69169 | 14: 64906621 | 69173 | 14: 65094231 | 69187 | 14: 65545962 |
| 69327 | 14: 68518147 | 69328 | 14: 68615752 | 69461 | 14: 72182566 | 69496 | 14: 72949159 | 69497 | 14: 72993679 |
| 69514 | 14: 73377613 | 69551 | 14: 74402579 | 69585 | 14: 75284663 | 69654 | 14: 76712535 | 69927 | 15: 278408 |
| 69928 | 15: 364919 | 69935 | 15: 581520 | 69938 | 15: 790710 | 69940 | 15: 921595 | 70011 | 15: 2907387 |
| 70072 | 15: 4118742 | 70298 | 15: 5868982 | 70344 | 15: 7132196 | 70468 | 15: 9855083 | 70479 | 15: 10238469 |
| 70485 | 15: 10450890 | 70487 | 15: 10557012 | 70500 | 15: 11034094 | 70513 | 15: 11544670 | 70515 | 15: 11646233 |
| 70535 | 15: 12337840 | 70536 | 15: 12426564 | 70558 | 15: 13317083 | 70564 | 15: 13540062 | 70607 | 15: 14553709 |
| 70611 | 15: 14685598 | 70652 | 15: 15637037 | 70768 | 15: 17775660 | 70799 | 15: 18549454 | 70821 | 15: 19204706 |
| 70830 | 15: 19463500 | 71142 | 15: 25686598 | 71348 | 15: 29961188 | 71383 | 15: 30812283 | 71652 | 15: 36545261 |
| 71720 | 15: 37976104 | 71736 | 15: 38441326 | 71771 | 15: 39152334 | 71772 | 15: 39251802 | 71782 | 15: 39467016 |
| 72064 | 15: 44893395 | 72102 | 15: 45703046 | 72116 | 15: 46016293 | 72117 | 15: 46052010 | 72118 | 15: 46128678 |
| 72120 | 15: 46190128 | 72193 | 15: 47315722 | 72195 | 15: 47377732 | 72196 | 15: 47473423 | 72199 | 15: 47598007 |
| 72204 | 15: 47732907 | 72264 | 15: 48782827 | 72293 | 15: 49903002 | 72297 | 15: 50131403 | 72298 | 15: 50228681 |
| 72321 | 15: 50928742 | 72446 | 15: 53225186 | 72482 | 15: 53576274 | 72512 | 15: 54128239 | 72554 | 15: 55076548 |
| 72599 | 15: 56354181 | 72600 | 15: 56425647 | 72782 | 15: 59421385 | 72830 | 15: 60713798 | 72933 | 15: 62959638 |
| 73204 | 15: 68655064 | 73251 | 15: 70097809 | 73252 | 15: 70187975 | 73651 | 15: 78826872 | 73652 | 15: 78926080 |
| 73661 | 15: 79238876 | 73664 | 15: 79380785 | 73669 | 15: 79544197 | 73670 | 15: 79611716 | 73785 | 15: 82130501 |
| 73795 | 15: 82404865 | 73796 | 15: 82425557 | 73797 | 15: 82522759 | 73884 | 15: 84032423 | 73887 | 15: 84244168 |
| 73888 | 15: 84260381 | 73889 | 15: 84356903 | 73890 | 15: 84447429 | 73891 | 15: 84540077 | 73894 | 15: 84686045 |
| 73895 | 15: 84729195 | 73896 | 15: 84804908 | 73897 | 15: 84896615 | 73898 | 15: 84939269 | 73901 | 16: 338529 |
| 7406 | 16: 3596934 | 74075 | 16: 3906912 | 74153 | 16: 5495502 | 74156 | 16: 5663689 | 74159 | 16: 5838621 |
| 74167 | 16: 6306111 | 74197 | 16: 6971134 | 74198 | 16: 7015739 | 74199 | 16: 7111115 | 74201 | 16: 7234019 |
| 74221 | 16: 7834741 | 74224 | 16: 7996266 | 74228 | 16: 8149785 | 74229 | 16: 8194291 | 74256 | 16: 9122788 |
| 74257 | 16: 9217866 | 74265 | 16: 9575503 | 74293 | 16: 10558307 | 74409 | 16: 13263254 | 74425 | 16: 13790388 |
| 74496 | 16: 15729876 | 74548 | 16: 17488453 | 74550 | 16: 17617156 | 74566 | 16: 18152045 | 74575 | 16: 18477012 |
| 74632 | 16: 19928511 | 74709 | 16: 21880848 | 74750 | 16: 22924908 | 74816 | 16: 24474002 | 74979 | 16: 27452942 |
| 75007 | 16: 28071735 | 75037 | 16: 29095796 | 75043 | 16: 29327042 | 75168 | 16: 32723586 | 75199 | 16: 33327718 |
| 75372 | 16: 37660699 | 75428 | 16: 38784562 | 75449 | 16: 39690828 | 75490 | 16: 40983811 | 75550 | 16: 42163425 |
| 75555 | 16: 42310324 | 75556 | 16: 42386477 | 75560 | 16: 42697916 | 75568 | 16: 42993444 | 75580 | 16: 43487631 |
| 75582 | 16: 43668103 | 75584 | 16: 43759140 | 75585 | 16: 43845132 | 75586 | 16: 43944562 | 75588 | 16: 44032421 |
| 75608 | 16: 44623635 | 75612 | 16: 44826869 | 75613 | 16: 44926372 | 75614 | 16: 45026368 | 75616 | 16: 45103219 |
| 75627 | 16: 45512990 | 75632 | 16: 45776402 | 75637 | 16: 46002437 | 75779 | 16: 49103242 | 75849 | 16: 51054365 |
| 75850 | 16: 51151596 | 75859 | 16: 51446273 | 75867 | 16: 51646308 | 75868 | 16: 51740534 | 75944 | 16: 53407745 |
| 75997 | 16: 54730701 | 76093 | 16: 56989855 | 76409 | 16: 64087780 | 76503 | 16: 65762070 | 76697 | 16: 69928868 |
| 76757 | 16: 71345017 | 76835 | 16: 72851827 | 77085 | 16: 77598464 | 77222 | 16: 79857865 | 77229 | 16: 80072357 |
| 77230 | 16: 80138235 | 77232 | 16: 80311616 | 77233 | 16: 80396660 | 77235 | 16: 80531024 | 77236 | 16: 80630465 |
| 77239 | 16: 80799166 | 77242 | 16: 80953675 | 77252 | 17: 259333 | 77263 | 17: 564310 | 77274 | 17: 973466 |
| 77278 | 17: 1054010 | 77487 | 17: 5568436 | 77620 | 17: 8228129 | 77681 | 17: 9733872 | 77735 | 17: 10897059 |
| 77736 | 17: 10938025 | 77737 | 17: 11020572 | 77738 | 17: 11094236 | 77755 | 17: 11494094 | 77830 | 17: 13277982 |
| 78070 | 17: 18481630 | 78136 | 17: 19808398 | 78137 | 17: 19897663 | 78207 | 17: 21967677 | 78230 | 17: 22786786 |
| 78249 | 17: 23511334 | 78255 | 17: 23800711 | 78322 | 17: 25936343 | 78401 | 17: 27754738 | 78430 | 17: 28426405 |
| 78441 | 17: 28791960 | 78462 | 17: 29447559 | 78487 | 17: 30217269 | 78545 | 17: 31620341 | 78567 | 17: 32254712 |
| 78570 | 17: 32296225 | 78667 | 17: 34617927 | 78688 | 17: 35304907 | 78690 | 17: 35390256 | 78721 | 17: 36229501 |
| 78734 | 17: 36837955 | 78744 | 17: 37134508 | 78795 | 17: 38760200 | 78814 | 17: 39443909 | 78936 | 17: 41933427 |
| 78937 | 17: 42031727 | 78986 | 17: 42956052 | 78987 | 17: 43050836 | 79027 | 17: 44108368 | 79108 | 17: 46082443 |
| 79178 | 17: 47678534 | 79293 | 17: 50425399 | 79393 | 17: 52219542 | 79410 | 17: 52792109 | 79481 | 17: 54629610 |
| 79511 | 17: 55298739 | 79535 | 17: 55992314 | 79545 | 17: 56246007 | 79980 | 17: 64743637 | 80106 | 17: 67144833 |
| 80130 | 17: 67822583 | 80279 | 17: 70683155 | 80280 | 17: 70709345 | 80281 | 17: 70709461 | 80291 | 17: 71094967 |
| 80383 | 17: 72831459 | 80395 | 18: 95447 | 80396 | 18: 189110 | 80398 | 18: 367703 | 80400 | 18: 514106 |
| 80908 | 18: 9808096 | 80994 | 18: 11474377 | 81022 | 18: 12273578 | 81074 | 18: 13692264 | 81098 | 18: 14306814 |
| 81099 | 18: 14400626 | 81103 | 18: 14549787 | 81105 | 18: 14605010 | 81106 | 18: 14636355 | 81107 | 18: 14731173 |
| 81108 | 18: 14801380 | 81151 | 18: 15872181 | 81153 | 18: 15986314 | 81159 | 18: 16182872 | 81377 | 18: 20732386 |
| 81736 | 18: 28718247 | 81811 | 18: 30779424 | 81813 | 18: 30921028 | 81837 | 18: 31639943 | 81954 | 18: 34144938 |
| 82045 | 18: 36256440 | 82052 | 18: 36555965 | 82152 | 18: 38719188 | 82233 | 18: 40460799 | 82285 | 18: 41570886 |
| 82432 | 18: 44652242 | 82443 | 18: 44987987 | 82600 | 18: 47190873 | 82671 | 18: 48450267 | 82706 | 18: 49271447 |
| 82707 | 18: 49370683 | 82738 | 18: 50083459 | 82759 | 18: 50626419 | 82760 | 18: 50725650 | 82837 | 18: 52240322 |
| 82862 | 18: 53044750 | 82869 | 18: 53322848 | 82997 | 18: 55515927 | 83109 | 18: 56926638 | 83227 | 18: 58451447 |
| 83237 | 18: 58961238 | 83240 | 18: 59153638 | 83249 | 18: 59423487 | 83250 | 18: 59502600 | 83251 | 18: 59597971 |
| 83252 | 18: 59640740 | 83253 | 18: 59738484 | 83254 | 18: 59752260 | 83255 | 18: 59841292 | 83271 | 18: 60187977 |
| 83273 | 18: 60349514 | 83293 | 18: 61084174 | 83294 | 18: 61180054 | 83301 | 18: 61392112 | 83302 | 18: 61484398 |
| 83373 | 18: 62547161 | 83374 | 18: 62548072 | 83375 | 18: 62548590 | 83376 | 18: 62614389 | 83386 | 18: 63006555 |
| 83387 | 18: 63077361 | 83422 | 18: 63662692 | 83588 | 19: 57925 | 83591 | 19: 305379 | 83596 | 19: 417884 |
| 83597 | 19: 509378 | 83599 | 19: 577296 | 83600 | 19: 668256 | 83629 | 19: 1655405 | 83656 | 19: 2558450 |
| 83665 | 19: 2953866 | 83671 | 19: 3120033 | 83677 | 19: 3275802 | 83714 | 19: 4397168 | 83980 | 19: 9821822 |
| 84056 | 19: 11385109 | 84253 | 19: 15470245 | 84549 | 19: 20561916 | 84554 | 19: 20740986 | 84572 | 19: 21295269 |
| 84638 | 19: 22476147 | 84674 | 19: 23274892 | 84679 | 19: 23407987 | 84688 | 19: 23703085 | 84976 | 19: 27081319 |
| 84977 | 19: 27125583 | 84978 | 19: 27182483 | 84979 | 19: 27275569 | 84986 | 19: 27665926 | 85115 | 19: 30350931 |
| 85176 | 19: 31793582 | 85191 | 19: 32153081 | 85511 | 19: 37954850 | 85578 | 19: 39940442 | 85649 | 19: 41359770 |
| 85749 | 19: 43284000 | 85860 | 19: 45535788 | 85910 | 19: 46199205 | 85919 | 19: 46495194 | 85936 | 19: 46990600 |
| 86031 | 19: 48855388 | 86072 | 19: 49706256 | 86111 | 19: 50303597 | 86113 | 19: 50431879 | 86114 | 19: 50531654 |
| 86241 | 19: 52131137 | 86250 | 19: 52418467 | 86565 | 19: 56677337 | 86621 | 19: 57762258 | 86622 | 19: 57849833 |
| 86961 | 20: 98572 | 86962 | 20: 194921 | 87107 | 20: 2918386 | 87430 | 20: 5583730 | 87593 | 20: 8760244 |
| 87840 | 20: 14544398 | 87842 | 20: 14662366 | 87845 | 20: 14842827 | 87846 | 20: 14894317 | 87848 | 20: 15043520 |
| 87850 | 20: 15190994 | 87888 | 20: 16233299 | 87915 | 20: 16830331 | 87920 | 20: 17017002 | 87956 | 20: 18017776 |
| 87971 | 20: 18426404 | 88037 | 20: 20313521 | 88073 | 20: 21317972 | 88231 | 20: 23439914 | 88321 | 20: 25028667 |
| 88322 | 20: 25092607 | 88327 | 20: 25369162 | 88338 | 20: 25827366 | 88357 | 20: 26444773 | 88371 | 20: 26712661 |
| 88493 | 20: 29517107 | 88527 | 20: 30497850 | 88541 | 20: 31003971 | 88598 | 20: 32503045 | 88599 | 20: 32522681 |
| 88600 | 20: 32622352 | 88602 | 20: 32778020 | 88695 | 20: 34788751 | 88842 | 20: 38032426 | 88884 | 20: 39213757 |
| 89063 | 20: 43010466 | 89065 | 20: 43140540 | 89068 | 20: 43254253 | 89073 | 20: 43352423 | 89105 | 20: 44220967 |
| 89127 | 20: 44701792 | 89143 | 20: 45276596 | 89180 | 20: 46098985 | 89181 | 20: 46185509 | 89205 | 20: 47109652 |
| 89218 | 20: 47365415 | 89224 | 20: 47651501 | 89232 | 20: 47954545 | 89237 | 20: 48129247 | 89238 | 20: 48218836 |
| 89257 | 20: 48716255 | 89281 | 20: 49326846 | 89283 | 20: 49443154 | 89319 | 20: 50486303 | 89360 | 20: 51882151 |
| 89375 | 20: 52355899 | 89376 | 20: 52454269 | 89400 | 20: 53180123 | 89438 | 20: 54174787 | 89439 | 20: 54272031 |
| 89441 | 20: 54375180 | 89450 | 20: 54601936 | 89464 | 20: 55005757 | 89492 | 20: 55642905 | 89496 | 20: 55834687 |
| 89710 | 20: 60274323 | 89746 | 20: 61511139 | 89881 | 20: 64307221 | 90177 | 20: 70744162 | 90271 | 20: 71587457 |
| 90278 | 20: 71850045 | 90283 | 21: 134737 | 90284 | 21: 233193 | 90286 | 21: 309646 | 90287 | 21: 371966 |
| 90288 | 21: 471326 | 90289 | 21: 506222 | 90291 | 21: 638478 | 90292 | 21: 726326 | 90293 | 21: 823686 |
| 90294 | 21: 904065 | 90295 | 21: 966923 | 90296 | 21: 1061609 | 90297 | 21: 1093204 | 90298 | 21: 1137598 |
| 90300 | 21: 1255004 | 90301 | 21: 1331973 | 90302 | 21: 1425976 | 90303 | 21: 1485463 | 90304 | 21: 1581245 |
| 90306 | 21: 1694504 | 90308 | 21: 1850823 | 90309 | 21: 1924655 | 90323 | 21: 2437601 | 90347 | 21: 3138288 |
| 90349 | 21: 3228959 | 90358 | 21: 3544839 | 90359 | 21: 3610217 | 90364 | 21: 3820824 | 90406 | 21: 4613245 |
| 90440 | 21: 5697639 | 90462 | 21: 6211650 | 90464 | 21: 6330904 | 90478 | 21: 6814441 | 90482 | 21: 6933120 |
| 90484 | 21: 7029661 | 90640 | 21: 10179845 | 91036 | 21: 19148704 | 91170 | 21: 21513912 | 91265 | 21: 23382552 |
| 91266 | 21: 23474228 | 91273 | 21: 23715679 | 91285 | 21: 24111132 | 91308 | 21: 24835888 | 91334 | 21: 25749419 |
| 91346 | 21: 26098112 | 91360 | 21: 26431821 | 91386 | 21: 27005517 | 91387 | 21: 27103067 | 91433 | 21: 28660586 |
| 91552 | 21: 31691093 | 91568 | 21: 32209086 | 91593 | 21: 32883760 | 91594 | 21: 32927345 | 91595 | 21: 33019785 |
| 91724 | 21: 34773624 | 91725 | 21: 34862867 | 91834 | 21: 37259953 | 91836 | 21: 37337560 | 91837 | 21: 37418367 |
| 91858 | 21: 38022062 | 91859 | 21: 38118482 | 91861 | 21: 38269475 | 91894 | 21: 39232808 | 91946 | 21: 40644524 |
| 91947 | 21: 40743815 | 91953 | 21: 40912381 | 91994 | 21: 41910649 | 92018 | 21: 42440438 | 92023 | 21: 42514187 |
| 92102 | 21: 44357068 | 92140 | 21: 44936824 | 92141 | 21: 45020642 | 92143 | 21: 45198066 | 92152 | 21: 45602636 |
| 92165 | 21: 46016604 | 92166 | 21: 46114894 | 92169 | 21: 46249176 | 92297 | 21: 48618715 | 92405 | 21: 51567065 |
| 92449 | 21: 52692952 | 92450 | 21: 52792942 | 92453 | 21: 52960457 | 92620 | 21: 56228179 | 92621 | 21: 56313681 |
| 92623 | 21: 56441767 | 92814 | 21: 59309517 | 92815 | 21: 59375264 | 93006 | 21: 63376882 | 93229 | 21: 67773042 |
| 93338 | 22: 94579 | 93352 | 22: 646933 | 93358 | 22: 824901 | 93437 | 22: 2467585 | 93677 | 22: 7210800 |
| 93714 | 22: 8230735 | 93734 | 22: 8758516 | 93765 | 22: 9634144 | 93849 | 22: 11365346 | 93942 | 22: 13343219 |
| 93966 | 22: 14036315 | 93997 | 22: 14787281 | 94092 | 22: 17058957 | 94135 | 22: 18197695 | 94162 | 22: 18799270 |
| 94176 | 22: 19116049 | 94364 | 22: 24074772 | 94470 | 22: 27069723 | 94481 | 22: 27482704 | 94563 | 22: 29220441 |
| 94595 | 22: 30012007 | 94650 | 22: 31491877 | 94657 | 22: 31704814 | 95074 | 22: 41624457 | 95174 | 22: 43984117 |
| 95228 | 22: 45235116 | 95236 | 22: 45525829 | 95393 | 22: 48902067 | 95902 | 22: 58335431 | 95953 | 22: 59355819 |
| 96035 | 23: 278582 | 96037 | 23: 430451 | 96042 | 23: 626975 | 96071 | 23: 1548151 | 96072 | 23: 1635007 |
| 96178 | 23: 3824609 | 96286 | 23: 5931144 | 96463 | 23: 9588241 | 96613 | 23: 12692439 | 96648 | 23: 13322760 |
| 96655 | 23: 13471246 | 96656 | 23: 13531584 | 97061 | 23: 21612939 | 97062 | 23: 21712441 | 97063 | 23: 21811769 |
| 97086 | 23: 22310386 | 97103 | 23: 22816656 | 97117 | 23: 23369505 | 97336 | 23: 25965936 | 97337 | 23: 26013871 |
| 97338 | 23: 26203805 | 97339 | 23: 26302825 | 97340 | 23: 26400435 | 97341 | 23: 26404617 | 97342 | 23: 26417922 |
| 97343 | 23: 26516930 | 97346 | 23: 26688460 | 97347 | 23: 26750001 | 97348 | 23: 26836934 | 97638 | 23: 30204175 |
| 97639 | 23: 30205302 | 97640 | 23: 30205793 | 97675 | 23: 30951436 | 97705 | 23: 31625189 | 97851 | 23: 34829880 |
| 97852 | 23: 34888702 | 97859 | 23: 35057295 | 97860 | 23: 35151436 | 97889 | 23: 36152667 | 97963 | 23: 38043115 |
| 98233 | 23: 42608340 | 98516 | 23: 48087560 | 98519 | 23: 48274159 | 98668 | 23: 50824134 | 98767 | 23: 52402327 |
| 98771 | 24: 201134 | 98932 | 24: 3597084 | 98987 | 24: 4929150 | 99002 | 24: 5203768 | 99108 | 24: 7965661 |
| 99109 | 24: 8063828 | 99131 | 24: 8583177 | 99304 | 24: 10935542 | 99358 | 24: 12240445 | 99425 | 24: 13974458 |
| 99478 | 24: 15604573 | 99486 | 24: 15989036 | 99487 | 24: 16079054 | 99561 | 24: 18229831 | 99572 | 24: 18541933 |
| 99573 | 24: 18640412 | 99606 | 24: 19585603 | 99618 | 24: 19942905 | 99624 | 24: 20173407 | 99688 | 24: 21131115 |
| 99764 | 24: 22214747 | 99765 | 24: 22312329 | 99816 | 24: 23615391 | 99822 | 24: 23975968 | 99826 | 24: 24145037 |
| 99942 | 24: 26860326 | 10005 | 24: 29671134 | 10010 | 24: 30883521 | 10010 | 24: 30966522 | 10021 | 24: 33423144 |
| 100223 | 24: 33544520 | 10033 | 24: 36290089 | 10034 | 24: 36532147 | 10039 | 24: 38166045 | 10040 | 24: 38422210 |
| 100411 | 24: 38722958 | 10047 | 24: 40089318 | 10047 | 24: 40188306 | 10054 | 24: 41297881 | 10062 | 24: 42939236 |
| 100630 | 24: 43395685 | 10064 | 24: 43924798 | 10064 | 24: 44019504 | 10079 | 24: 46654829 | 10085 | 24: 48033977 |
| 100863 | 24: 48144296 | 10087 | 24: 48567652 | 10088 | 24: 48858324 | 10101 | 24: 52201248 | 10105 | 24: 52902393 |
| 101118 | 24: 54423570 | 10118 | 24: 55890705 | 10120 | 24: 56201929 | 10149 | 24: 61932447 | 10150 | 24: 62180714 |
| 101504 | 24: 62277921 | 10150 | 25: 94971 | 10161 | 25: 2097812 | 10174 | 25: 3066063 | 10199 | 25: 7620341 |
| 102020 | 25: 8255600 | 10204 | 25: 8952928 | 10205 | 25: 9079254 | 10228 | 25: 14296254 | 10246 | 25: 18216204 |
| 102662 | 25: 22863656 | 10269 | 25: 23717883 | 10285 | 25: 26775859 | 10304 | 25: 30451801 | 10304 | 25: 30672143 |
| 103152 | 25: 32745193 | 10360 | 25: 40515689 | 10370 | 26: 71749 | 10370 | 26: 134754 | 10370 | 26: 202644 |
| 103710 | 26: 292215 | 10371 | 26: 332815 | 10371 | 26: 418885 | 10371 | 26: 467022 | 10371 | 26: 552651 |
| 103715 | 26: 648778 | 10372 | 26: 841636 | 10372 | 26: 933342 | 10374 | 26: 1410312 | 10376 | 26: 2231655 |
| 103788 | 26: 2771137 | 10388 | 26: 4804367 | 10390 | 26: 5316048 | 10391 | 26: 5782436 | 10402 | 26: 8899248 |
| 104047 | 26: 9422315 | 10405 | 26: 9629233 | 10413 | 26: 11253125 | 10413 | 26: 11345317 | 10413 | 26: 11428247 |
| 104158 | 26: 11886123 | 10426 | 26: 14376252 | 10428 | 26: 15092211 | 10428 | 26: 15180609 | 10432 | 26: 16278407 |
| 104330 | 26: 16409629 | 10433 | 26: 16616504 | 10448 | 26: 19837144 | 10449 | 26: 20063209 | 10450 | 26: 20330629 |
| 104526 | 26: 21059250 | 10461 | 26: 23492556 | 10462 | 26: 23837564 | 10463 | 26: 23968846 | 10481 | 26: 27340392 |
| 104865 | 26: 28704657 | 10492 | 26: 30296052 | 10493 | 26: 30555507 | 10496 | 26: 31506273 | 10510 | 26: 34494728 |
| 105234 | 26: 37659896 | 10530 | 26: 39301430 | 10532 | 26: 39903212 | 10538 | 26: 41736203 | 10542 | 26: 42498087 |
| 105426 | 26: 42598014 | 10545 | 26: 43157974 | 10552 | 26: 44983809 | 10558 | 26: 45965280 | 10575 | 26: 49691984 |
| 105779 | 26: 50189494 | 10578 | 26: 50197785 | 10578 | 26: 50203962 | 10578 | 26: 50261536 | 10578 | 26: 50350367 |
| 105784 | 26: 50350409 | 10578 | 26: 50414892 | 10585 | 27: 99932 | 10585 | 27: 181641 | 10585 | 27: 250836 |
| 105858 | 27: 316731 | 10585 | 27: 394123 | 10586 | 27: 443903 | 10586 | 27: 543122 | 10586 | 27: 642666 |
| 105867 | 27: 739223 | 10586 | 27: 838134 | 10586 | 27: 928860 | 10587 | 27: 1021674 | 10587 | 27: 1119724 |
| 105923 | 27: 1865958 | 10599 | 27: 3535767 | 10599 | 27: 3755867 | 10611 | 27: 6215443 | 10611 | 27: 6337782 |
| 106121 | 27: 6434403 | 10612 | 27: 6434442 | 10612 | 27: 6621177 | 10612 | 27: 6714941 | 10612 | 27: 6754631 |
| 106126 | 27: 6761335 | 10612 | 27: 6952355 | 10612 | 27: 6962879 | 10612 | 27: 6980835 | 10613 | 27: 6994129 |
| 106250 | 27: 9301634 | 10626 | 27: 9835607 | 10627 | 27: 10239566 | 10632 | 27: 11805253 | 10633 | 27: 12112133 |
| 106347 | 27: 12322130 | 10634 | 27: 12386638 | 10645 | 27: 14207575 | 10665 | 27: 18065646 | 10666 | 27: 18151157 |
| 106710 | 27: 19373108 | 10676 | 27: 20532211 | 10677 | 27: 20833561 | 10682 | 27: 22102480 | 10682 | 27: 22221678 |
| 106829 | 27: 22350322 | 10684 | 27: 22785446 | 10699 | 27: 26185311 | 10711 | 27: 28416757 | 10718 | 27: 30124180 |
| 107366 | 27: 33574896 | 10737 | 27: 34012998 | 10747 | 27: 35694726 | 10753 | 27: 36991890 | 10762 | 27: 38609999 |
| 107654 | 27: 39285504 | 10789 | 27: 44800138 | 10791 | 27: 45569924 | 10792 | 28: 39225 | 10792 | 28: 138659 |
| 107922 | 28: 231989 | 10792 | 28: 285448 | 10792 | 28: 328039 | 10792 | 28: 386583 | 10792 | 28: 563052 |
| 107930 | 28: 662354 | 10793 | 28: 794289 | 10793 | 28: 893452 | 10801 | 28: 2643580 | 10802 | 28: 2904232 |
| 108027 | 28: 2999388 | 10803 | 28: 3161546 | 10803 | 28: 3253315 | 10803 | 28: 3405359 | 10804 | 28: 3759573 |
| 108043 | 28: 3787485 | 10804 | 28: 3885321 | 10804 | 28: 4073833 | 10804 | 28: 4163374 | 10805 | 28: 4253300 |
| 108051 | 28: 4338728 | 10805 | 28: 4513285 | 10806 | 28: 5004251 | 10818 | 28: 7591484 | 10827 | 28: 9724722 |
| 108313 | 28: 10660380 | 10832 | 28: 11111391 | 10832 | 28: 11222161 | 10843 | 28: 13814734 | 10854 | 28: 16313184 |
| 108613 | 28: 17683321 | 10863 | 28: 18306254 | 10874 | 28: 21104115 | 10877 | 28: 22035988 | 10879 | 28: 22784794 |
| 108801 | 28: 22871308 | 10880 | 28: 22889009 | 10884 | 28: 23719431 | 10884 | 28: 23801384 | 10902 | 28: 27215263 |
| 109182 | 28: 30404928 | 10933 | 28: 33475488 | 10938 | 28: 34421752 | 10943 | 28: 35588714 | 10949 | 28: 36727428 |
| 109624 | 28: 39439768 | 10962 | 28: 39494932 | 10963 | 28: 39750515 | 10993 | 28: 44515897 | 11000 | 29: 178851 |
| 110009 | 29: 265250 | 11001 | 29: 607202 | 11007 | 29: 1956188 | 11008 | 29: 2091012 | 11023 | 29: 5374583 |
| 110238 | 29: 5456512 | 11023 | 29: 5547355 | 11024 | 29: 5627071 | 11024 | 29: 5698810 | 11028 | 29: 7011954 |
| 110333 | 29: 8334191 | 11033 | 29: 8529992 | 11054 | 29: 13277373 | 11055 | 29: 13442800 | 11058 | 29: 14334295 |
| 110592 | 29: 14552146 | 11066 | 29: 16276745 | 11073 | 29: 18298255 | 11074 | 29: 18821433 | 11074 | 29: 19176738 |
| 110756 | 29: 19577395 | 11081 | 29: 20938903 | 11081 | 29: 21062969 | 11084 | 29: 21741949 | 11102 | 29: 26286827 |
| 111040 | 29: 26637610 | 11104 | 29: 26812835 | 11105 | 29: 27123234 | 11105 | 29: 27286001 | 11106 | 29: 27336935 |
| 111061 | 29: 27426724 | 11138 | 29: 34145956 | 11151 | 29: 36681488 | 11156 | 29: 37610625 | 11157 | 29: 37814400 |
| 111573 | 29: 37903323 | 11157 | 29: 37975002 | 11157 | 29: 38065526 | 11157 | 29: 38161198 | 11157 | 29: 38251275 |
| 111578 | 29: 38348064 | 11157 | 29: 38414602 | 11158 | 29: 38450767 | 11158 | 29: 38467768 | 11158 | 29: 38556318 |
| 111583 | 29: 38649308 | 11158 | 29: 38752213 | 11158 | 29: 38847684 | 11158 | 29: 38917900 | 11158 | 29: 38952660 |
| 111590 | 29: 38993981 | 11159 | 29: 39083787 | 11159 | 29: 39205805 | 11172 | 29: 41919049 | 11214 | 29: 50420995 |
| 112179 | 29: 50977672 | 11218 | 29: 51071432. | | | | | | |

sites of the seventh marker panel are shown in the following table:

13. A method for genomic selection (GS) breeding of Huaxi cattle using a genome-wide breeding chip, wherein the genome-wide breeding chip of Huaxi cattle comprises a molecular marker combination;
wherein the molecular marker combination comprises seven marker panels; a first marker panel comprising 7,221 SNPs; a second marker panel comp comprising rises 22,937 SNPs and 1 Indel; a third marker panel comprising 249 SNPs; a fourth marker panel comprising 3,907 SNPs and 2 Indels; a fifth marker panel comprising 6,190 SNPs; a sixth marker panel comprising a total of 74,098 markers verified in a Huaxi cattle population from Illumina BovineHD chip and Neogen GGP Bovine 100K chip; a seventh marker panel comprising 2,617 SNPs; and an ARS-UCD 1.2/bosTau9 version is used as a reference genome of cattle;
the sites of the first marker panel are shown in the following table:

| NO. | Position | NO. | Position | NO. | Position | NO. | Position | NO. | Position |
|---|---|---|---|---|---|---|---|---|---|
| 27 | 1: 1073001 | 40 | 1: 1271108 | 124 | 1: 3014460 | 164 | 1: 3598559 | 213 | 1: 4312806 |
| 216 | 1: 4341656 | 257 | 1: 5479286 | 286 | 1: 6042789 | 314 | 1: 6638827 | 324 | 1: 6809931 |
| 386 | 1: 8222113 | 399 | 1: 8645362 | 407 | 1: 8747963 | 480 | 1: 10381445 | 484 | 1: 10442662 |
| 547 | 1: 12065998 | 575 | 1: 12850062 | 586 | 1: 13291614 | 593 | 1: 13478803 | 594 | 1: 13478804 |
| 595 | 1: 13478805 | 600 | 1: 13618910 | 602 | 1: 13649839 | 603 | 1: 13650350 | 604 | 1: 13650573 |
| 605 | 1: 13650578 | 606 | 1: 13657524 | 607 | 1: 13657759 | 608 | 1: 13657853 | 609 | 1: 13659104 |
| 610 | 1: 13659735 | 611 | 1: 13660313 | 612 | 1: 13662505 | 613 | 1: 13662879 | 614 | 1: 13663313 |
| 615 | 1: 13663369 | 632 | 1: 14229816 | 635 | 1: 14367624 | 640 | 1: 14444223 | 646 | 1: 14671195 |
| 675 | 1: 15289219 | 732 | 1: 17104389 | 745 | 1: 17433162 | 761 | 1: 18058788 | 779 | 1: 18612790 |
| 803 | 1: 19491923 | 807 | 1: 19546032 | 831 | 1: 20249479 | 855 | 1: 20731028 | 866 | 1: 21118209 |
| 901 | 1: 22054771 | 936 | 1: 23011477 | 954 | 1: 23715328 | 957 | 1: 23772692 | 974 | 1: 24145046 |
| 1014 | 1: 25285394 | 1015 | 1: 25305166 | 1018 | 1: 25448644 | 1038 | 1: 25974291 | 1075 | 1: 26927446 |
| 1089 | 1: 27357800 | 1132 | 1: 29040826 | 1142 | 1: 29268481 | 1173 | 1: 30001509 | 1176 | 1: 30051285 |
| 1177 | 1: 30052889 | 1206 | 1: 31087002 | 1207 | 1: 31090461 | 1211 | 1: 31197827 | 1243 | 1: 32461661 |
| 1255 | 1: 32885018 | 1263 | 1: 33160247 | 1384 | 1: 36327051 | 1385 | 1: 36335560 | 1404 | 1: 36719216 |
| 1410 | 1: 36920529 | 1412 | 1: 36984896 | 1458 | 1: 38181926 | 1480 | 1: 38770057 | 1483 | 1: 38899433 |
| 1495 | 1: 39263861 | 1539 | 1: 40637359 | 1546 | 1: 40754498 | 1571 | 1: 41666745 | 1617 | 1: 42491489 |
| 1667 | 1: 43750606 | 1687 | 1: 44125197 | 1691 | 1: 44260960 | 1710 | 1: 44679137 | 1713 | 1: 44805494 |
| 1719 | 1: 44925165 | 1720 | 1: 44925173 | 1721 | 1: 44925749 | 1722 | 1: 44927653 | 1723 | 1: 44928339 |
| 1724 | 1: 44928350 | 1725 | 1: 44928489 | 1726 | 1: 44929514 | 1727 | 1: 44929625 | 1728 | 1: 44929973 |
| 1729 | 1: 44931081 | 1730 | 1: 44931463 | 1753 | 1: 45241871 | 1820 | 1: 47384886 | 1828 | 1: 47617677 |
| 1841 | 1: 48116097 | 1963 | 1: 52181284 | 1995 | 1: 53016532 | 2003 | 1: 53237486 | 2005 | 1: 53252387 |
| 2006 | 1: 53260493 | 2009 | 1: 53326573 | 2013 | 1: 53415161 | 2016 | 1: 53539734 | 2030 | 1: 53652726 |
| 2044 | 1: 53770698 | 2051 | 1: 54027097 | 2052 | 1: 54039879 | 2053 | 1: 54067827 | 2054 | 1: 54068465 |
| 2062 | 1: 54170560 | 2063 | 1: 54175315 | 2073 | 1: 54409914 | 2077 | 1: 54504340 | 2078 | 1: 54507288 |
| 2094 | 1: 54985100 | 2100 | 1: 55132890 | 2113 | 1: 55602206 | 2115 | 1: 55658614 | 2130 | 1: 56173438 |
| 2133 | 1: 56288769 | 2135 | 1: 56366295 | 2136 | 1: 56367389 | 2137 | 1: 56367471 | 2140 | 1: 56429824 |
| 2141 | 1: 56439077 | 2151 | 1: 56684792 | 2155 | 1: 56767210 | 2164 | 1: 56950851 | 2239 | 1: 58810751 |
| 2254 | 1: 59120478 | 2289 | 1: 60052451 | 2299 | 1: 60321205 | 2305 | 1: 60534555 | 2314 | 1: 60643904 |
| 2322 | 1: 60871605 | 2331 | 1: 61098681 | 2344 | 1: 61320487 | 2345 | 1: 61335565 | 2350 | 1: 61476653 |
| 2374 | 1: 62211191 | 2410 | 1: 62916376 | 2411 | 1: 62918132 | 2415 | 1: 63017753 | 2416 | 1: 63029817 |
| 2417 | 1: 63033101 | 2418 | 1: 63035068 | 2419 | 1: 63037531 | 2420 | 1: 63038112 | 2421 | 1: 63038183 |
| 2422 | 1: 63050131 | 2424 | 1: 63119198 | 2425 | 1: 63125457 | 2426 | 1: 63128635 | 2427 | 1: 63130293 |
| 2436 | 1: 63393320 | 2450 | 1: 63809663 | 2531 | 1: 65592899 | 2574 | 1: 66873010 | 2577 | 1: 67103986 |
| 2584 | 1: 67236736 | 2585 | 1: 67260968 | 2636 | 1: 68655539 | 2642 | 1: 68743594 | 2664 | 1: 68997540 |
| 2707 | 1: 69843446 | 2722 | 1: 70196536 | 2764 | 1: 70655375 | 2776 | 1: 70889623 | 2777 | 1: 70925120 |
| 2811 | 1: 71906639 | 2821 | 1: 72100009 | 2896 | 1: 73841520 | 2929 | 1: 74779617 | 2950 | 1: 75412538 |
| 2963 | 1: 75770166 | 2964 | 1: 75770197 | 2965 | 1: 75770199 | 2966 | 1: 75783854 | 2971 | 1: 75829393 |
| 2984 | 1: 76114883 | 3005 | 1: 76860758 | 3042 | 1: 77680697 | 3045 | 1: 77701368 | 3070 | 1: 78334604 |
| 3071 | 1: 78335278 | 3075 | 1: 78397021 | 3113 | 1: 79354013 | 3224 | 1: 81621771 | 3225 | 1: 81658300 |
| 3262 | 1: 82505239 | 3290 | 1: 83265388 | 3301 | 1: 83612627 | 3349 | 1: 85328309 | 3362 | 1: 85790627 |
| 3458 | 1: 88779015 | 3466 | 1: 88853263 | 3483 | 1: 89288724 | 3506 | 1: 89791299 | 3511 | 1: 89828935 |
| 3523 | 1: 90054999 | 3528 | 1: 90169751 | 3539 | 1: 90383988 | 3572 | 1: 91171394 | 3584 | 1: 91442961 |
| 3603 | 1: 91945075 | 3608 | 1: 92172948 | 3613 | 1: 92213222 | 3618 | 1: 92387065 | 3635 | 1: 93023669 |
| 3663 | 1: 93808595 | 3690 | 1: 94443181 | 3742 | 1: 95761493 | 3774 | 1: 96387293 | 3818 | 1: 97495906 |
| 3831 | 1: 97875563 | 3835 | 1: 97919421 | 3848 | 1: 98319004 | 4084 | 1: 105587286 | 4086 | 1: 105625485 |
| 4088 | 1: 105653161 | 4093 | 1: 105729523 | 4163 | 1: 107822647 | 4184 | 1: 108388088 | 4193 | 1: 108564048 |
| 4221 | 1: 109261833 | 4235 | 1: 109562630 | 4257 | 1: 110017436 | 4317 | 1: 111454247 | 4321 | 1: 111612080 |
| 4346 | 1: 112243802 | 4358 | 1: 112481363 | 4360 | 1: 112532754 | 4385 | 1: 112920549 | 4398 | 1: 113309692 |
| 4428 | 1: 113860732 | 4469 | 1: 114867292 | 4471 | 1: 114870124 | 4500 | 1: 115756884 | 4501 | 1: 115772405 |
| 4512 | 1: 116396361 | 4572 | 1: 117632176 | 4674 | 1: 119359964 | 4675 | 1: 119363741 | 4707 | 1: 120254924 |
| 4715 | 1: 120408319 | 4716 | 1: 120409192 | 4727 | 1: 120576333 | 4737 | 1: 120851703 | 4754 | 1: 121481221 |
| 4758 | 1: 121544917 | 4856 | 1: 124230559 | 4872 | 1: 124533841 | 4873 | 1: 124538558 | 4889 | 1: 124936627 |
| 4930 | 1: 125655889 | 4933 | 1: 125689194 | 4936 | 1: 125738236 | 4941 | 1: 125837099 | 4945 | 1: 125909016 |
| 4952 | 1: 126088394 | 5002 | 1: 127555264 | 5013 | 1: 127861573 | 5035 | 1: 128339939 | 5048 | 1: 128645880 |
| 5056 | 1: 128748369 | 5218 | 1: 131754936 | 5233 | 1: 132161523 | 5273 | 1: 133625347 | 5304 | 1: 134472909 |
| 5333 | 1: 135062430 | 5365 | 1: 135657617 | 5404 | 1: 135977700 | 5417 | 1: 136372646 | 5458 | 1: 137985176 |
| 5493 | 1: 139072937 | 5525 | 1: 140006988 | 5547 | 1: 140601043 | 5559 | 1: 140911883 | 5568 | 1: 141070658 |
| 5577 | 1: 141182724 | 5635 | 1: 142058648 | 5639 | 1: 142168313 | 5683 | 1: 142908923 | 5952 | 1: 148034123 |
| 5953 | 1: 148040606 | 5954 | 1: 148040677 | 5955 | 1: 148044952 | 5956 | 1: 148046284 | 5957 | 1: 148046462 |
| 5958 | 1: 148047932 | 5959 | 1: 148053077 | 5960 | 1: 148055822 | 5961 | 1: 148058260 | 5962 | 1: 148073345 |
| 5963 | 1: 148073444 | 5964 | 1: 148075396 | 5974 | 1: 148329401 | 5980 | 1: 148487864 | 6038 | 1: 149409627 |
| 6039 | 1: 149411937 | 6040 | 1: 149413628 | 6045 | 1: 149560052 | 6060 | 1: 149819819 | 6139 | 1: 151376946 |
| 6140 | 1: 151406584 | 6141 | 1: 151407059 | 6142 | 1: 151407604 | 6152 | 1: 151612724 | 6169 | 1: 151767739 |
| 6171 | 1: 151799413 | 6172 | 1: 151799855 | 6173 | 1: 151799970 | 6174 | 1: 151800242 | 6175 | 1: 151801164 |
| 6176 | 1: 151801587 | 6177 | 1: 151801737 | 6178 | 1: 151801752 | 6191 | 1: 152091262 | 6201 | 1: 152265191 |
| 6202 | 1: 152266056 | 6203 | 1: 152266869 | 6204 | 1: 152267613 | 6205 | 1: 152267787 | 6206 | 1: 152268300 |
| 6207 | 1: 152268458 | 6213 | 1: 152339615 | 6214 | 1: 152343252 | 6216 | 1: 152348515 | 6217 | 1: 152349140 |
| 6219 | 1: 152350298 | 6220 | 1: 152351423 | 6221 | 1: 152353474 | 6222 | 1: 152353711 | 6223 | 1: 152353915 |
| 6224 | 1: 152354049 | 6225 | 1: 152354217 | 6226 | 1: 152354593 | 6227 | 1: 152354711 | 6228 | 1: 152355277 |
| 6229 | 1: 152355296 | 6230 | 1: 152355677 | 6231 | 1: 152363064 | 6236 | 1: 152422417 | 6266 | 1: 152974350 |
| 6287 | 1: 153459144 | 6356 | 1: 154782431 | 6390 | 1: 155509473 | 6395 | 1: 155617067 | 6397 | 1: 155639845 |
| 6398 | 1: 155669488 | 6406 | 1: 155709356 | 6410 | 1: 155739527 | 6415 | 1: 155837092 | 6420 | 1: 155936300 |
| 6426 | 1: 156024716 | 6437 | 1: 156119189 | 6463 | 1: 156640964 | 6466 | 1: 156695097 | 6470 | 1: 156760308 |
| 6486 | 1: 157055516 | 6504 | 1: 157454125 | 6507 | 1: 157551305 | 6553 | 2: 894974 | 6604 | 2: 1577280 |
| 6679 | 2: 3866542 | 6686 | 2: 3991289 | 6688 | 2: 4033904 | 6690 | 2: 4040966 | 6691 | 2: 4053609 |
| 6707 | 2: 4311756 | 6747 | 2: 5066365 | 6793 | 2: 5931885 | 6795 | 2: 5954528 | 6847 | 2: 6974699 |
| 6858 | 2: 7193712 | 6872 | 2: 7370181 | 6879 | 2: 7526996 | 6881 | 2: 7574742 | 6942 | 2: 9325130 |
| 6974 | 2: 10332348 | 7018 | 2: 11668736 | 7023 | 2: 11736825 | 7056 | 2: 13064383 | 7074 | 2: 13460557 |
| 7080 | 2: 13648131 | 7107 | 2: 14339353 | 7122 | 2: 14701151 | 7166 | 2: 15712399 | 7233 | 2: 17570676 |
| 7234 | 2: 17577794 | 7308 | 2: 18634499 | 7318 | 2: 18907347 | 7319 | 2: 18942177 | 7320 | 2: 18943474 |
| 7321 | 2: 18951656 | 7322 | 2: 18958579 | 7323 | 2: 18964610 | 7324 | 2: 18967934 | 7333 | 2: 19110921 |
| 7382 | 2: 20297370 | 7385 | 2: 20378241 | 7392 | 2: 20658754 | 7396 | 2: 20683165 | 7424 | 2: 21454390 |
| 7453 | 2: 22063850 | 7454 | 2: 22086957 | 7457 | 2: 22138573 | 7515 | 2: 23573584 | 7538 | 2: 24013539 |
| 7549 | 2: 24246589 | 7565 | 2: 24496758 | 7566 | 2: 24500311 | 7567 | 2: 24524614 | 7570 | 2: 24563421 |
| 7588 | 2: 24952433 | 7603 | 2: 25454747 | 7645 | 2: 26254049 | 7656 | 2: 26496144 | 7725 | 2: 27887225 |
| 7734 | 2: 28111908 | 7757 | 2: 28481427 | 7809 | 2: 29740566 | 7881 | 2: 31547829 | 7957 | 2: 33826799 |
| 8010 | 2: 35264773 | 8019 | 2: 35614289 | 8041 | 2: 36281226 | 8076 | 2: 37317121 | 8084 | 2: 37618680 |
| 8086 | 2: 37625413 | 8229 | 2: 41341923 | 8233 | 2: 41443343 | 8234 | 2: 41451445 | 8235 | 2: 41463995 |
| 8236 | 2: 41476600 | 8239 | 2: 41525245 | 8240 | 2: 41526634 | 8241 | 2: 41546403 | 8242 | 2: 41555839 |
| 8243 | 2: 41555881 | 8244 | 2: 41556165 | 8248 | 2: 41609088 | 8249 | 2: 41648397 | 8263 | 2: 42040167 |
| 8264 | 2: 42120013 | 8283 | 2: 42489642 | 8284 | 2: 42493609 | 8285 | 2: 42495164 | 8313 | 2: 43227689 |
| 8369 | 2: 44933199 | 8396 | 2: 45615535 | 8398 | 2: 45632178 | 8409 | 2: 45890270 | 8416 | 2: 46041603 |
| 8493 | 2: 47767849 | 8507 | 2: 48158383 | 8511 | 2: 48296454 | 8537 | 2: 48966694 | 8542 | 2: 48995422 |
| 8546 | 2: 49074113 | 8587 | 2: 50450300 | 8631 | 2: 51841153 | 8632 | 2: 51841281 | 8633 | 2: 51845196 |
| 8634 | 2: 51846388 | 8635 | 2: 51848506 | 8636 | 2: 51849168 | 8637 | 2: 51850817 | 8638 | 2: 51851321 |
| 8708 | 2: 53799255 | 8721 | 2: 54148820 | 8743 | 2: 54689808 | 8751 | 2: 54852723 | 8752 | 2: 54874680 |
| 8786 | 2: 55757496 | 8788 | 2: 55856268 | 8836 | 2: 57348471 | 8850 | 2: 57670111 | 8867 | 2: 58206752 |
| 8868 | 2: 58209897 | 8870 | 2: 58220436 | 8871 | 2: 58224932 | 8872 | 2: 58235017 | 8895 | 2: 58894479 |
| 8937 | 2: 59886642 | 8939 | 2: 59894079 | 8941 | 2: 59895422 | 8953 | 2: 60262781 | 8986 | 2: 61265032 |
| 9027 | 2: 62337521 | 9064 | 2: 63236556 | 9082 | 2: 63698530 | 9103 | 2: 64357206 | 9104 | 2: 64367151 |
| 9106 | 2: 64369812 | 9108 | 2: 64437590 | 9109 | 2: 64438167 | 9110 | 2: 64438313 | 9111 | 2: 64438576 |
| 9112 | 2: 64438580 | 9113 | 2: 64438635 | 9114 | 2: 64438908 | 9122 | 2: 64709919 | 9123 | 2: 64711805 |
| 9144 | 2: 65152676 | 9170 | 2: 65933901 | 9210 | 2: 67245827 | 9238 | 2: 68102588 | 9241 | 2: 68162816 |
| 9257 | 2: 68641959 | 9282 | 2: 69654612 | 9286 | 2: 69797972 | 9289 | 2: 69913137 | 9309 | 2: 70434838 |
| 9326 | 2: 70835144 | 9351 | 2: 71366915 | 9354 | 2: 71385196 | 9380 | 2: 72368600 | 9431 | 2: 73656519 |
| 9432 | 2: 73692943 | 9438 | 2: 73821323 | 9450 | 2: 74104600 | 9460 | 2: 74465077 | 9499 | 2: 75629903 |
| 9522 | 2: 76151639 | 9528 | 2: 76318715 | 9572 | 2: 77446117 | 9613 | 2: 78673595 | 9630 | 2: 78917434 |
| 9682 | 2: 80074128 | 9733 | 2: 82257633 | 9802 | 2: 84430538 | 9875 | 2: 86138420 | 9884 | 2: 86394912 |
| 10120 | 2: 91927539 | 10135 | 2: 92191854 | 10175 | 2: 93130350 | 10210 | 2: 93703916 | 10252 | 2: 94676418 |
| 10283 | 2: 95426880 | 10287 | 2: 95450849 | 10300 | 2: 95820889 | 10323 | 2: 96340062 | 10366 | 2: 97323100 |
| 10378 | 2: 97726887 | 10382 | 2: 97746848 | 10396 | 2: 98170549 | 10402 | 2: 98356021 | 10418 | 2: 98570804 |
| 10421 | 2: 98681146 | 10424 | 2: 98710984 | 10458 | 2: 99816412 | 10462 | 2: 99892472 | 10475 | 2: 100046086 |
| 10476 | 2: 100060098 | 10482 | 2: 100180128 | 10493 | 2: 100545847 | 10503 | 2: 100804647 | 10519 | 2: 101319592 |
| 10560 | 2: 102476291 | 10585 | 2: 103244299 | 10601 | 2: 103704057 | 10604 | 2: 103752303 | 10606 | 2: 103798942 |
| 10619 | 2: 104096932 | 10723 | 2: 106585047 | 10741 | 2: 106795862 | 10757 | 2: 107198319 | 10759 | 2: 107259324 |
| 10775 | 2: 107639270 | 10795 | 2: 107803660 | 10816 | 2: 108400383 | 10844 | 2: 108979070 | 10883 | 2: 109843763 |
| 10889 | 2: 109962178 | 10898 | 2: 110266270 | 10908 | 2: 110533827 | 10913 | 2: 110782529 | 10921 | 2: 111164890 |
| 10941 | 2: 111820434 | 10980 | 2: 112795307 | 11039 | 2: 113925261 | 11047 | 2: 114161924 | 11082 | 2: 115099463 |
| 11086 | 2: 115273767 | 11117 | 2: 115778796 | 11118 | 2: 115812040 | 11122 | 2: 115883386 | 11129 | 2: 116006798 |
| 11136 | 2: 116182220 | 11144 | 2: 116499676 | 11175 | 2: 117182401 | 11219 | 2: 118492898 | 11223 | 2: 118587365 |
| 11228 | 2: 118743982 | 11256 | 2: 119406723 | 11265 | 2: 119542337 | 11296 | 2: 120281653 | 11318 | 2: 120899441 |
| 11346 | 2: 121587712 | 11400 | 2: 123193388 | 11465 | 2: 125068403 | 11477 | 2: 125260143 | 11482 | 2: 125405078 |
| 11524 | 2: 126369897 | 11535 | 2: 126645449 | 11566 | 2: 126956287 | 11600 | 2: 127060935 | 11611 | 2: 127195966 |
| 11616 | 2: 127259539 | 11643 | 2: 127688853 | 11647 | 2: 127843626 | 11660 | 2: 128258137 | 11661 | 2: 128269460 |
| 11739 | 2: 129924952 | 11741 | 2: 130000075 | 11795 | 2: 131203320 | 11796 | 2: 131203333 | 11797 | 2: 131206099 |
| 11802 | 2: 131272766 | 11848 | 2: 132287345 | 11863 | 2: 132630938 | 11864 | 2: 132635825 | 11865 | 2: 132637425 |
| 11922 | 2: 133672767 | 11948 | 2: 133927517 | 11962 | 2: 134187671 | 11976 | 2: 134370377 | 12003 | 2: 134731903 |
| 12072 | 2: 135676512 | 12100 | 3: 74989 | 12107 | 3: 258342 | 12138 | 3: 907384 | 12182 | 3: 1660018 |
| 12186 | 3: 1722820 | 12267 | 3: 3478015 | 12291 | 3: 4097674 | 12293 | 3: 4100027 | 12301 | 3: 4206473 |
| 12342 | 3: 4711535 | 12358 | 3: 4936089 | 12364 | 3: 5121045 | 12365 | 3: 5121673 | 12366 | 3: 5126160 |
| 12367 | 3: 5130659 | 12386 | 3: 5499346 | 12429 | 3: 6474877 | 12436 | 3: 6635006 | 12448 | 3: 6773779 |
| 12474 | 3: 7163463 | 12482 | 3: 7308711 | 12520 | 3: 7916080 | 12530 | 3: 7997693 | 12589 | 3: 9507918 |
| 12593 | 3: 9562991 | 12600 | 3: 9657041 | 12615 | 3: 9990546 | 12621 | 3: 10103527 | 12678 | 3: 10496754 |
| 12726 | 3: 11145282 | 12768 | 3: 12417249 | 12776 | 3: 12650225 | 12779 | 3: 12749123 | 12804 | 3: 13251625 |
| 12806 | 3: 13370163 | 12807 | 3: 13373604 | 12808 | 3: 13375404 | 12812 | 3: 13573971 | 12833 | 3: 13902991 |
| 12866 | 3: 14463788 | 12898 | 3: 15197212 | 12926 | 3: 15692187 | 12941 | 3: 15905069 | 12972 | 3: 16573676 |
| 12993 | 3: 16936423 | 13136 | 3: 21101166 | 13141 | 3: 21359229 | 13158 | 3: 21799171 | 13166 | 3: 21978447 |
| 13170 | 3: 22029801 | 13230 | 3: 23274662 | 13295 | 3: 24786196 | 13318 | 3: 25281477 | 13329 | 3: 25568097 |
| 13394 | 3: 26947828 | 13404 | 3: 27140090 | 13421 | 3: 27417464 | 13426 | 3: 27531689 | 13432 | 3: 27586695 |
| 13433 | 3: 27587568 | 13434 | 3: 27588165 | 13435 | 3: 27590766 | 13436 | 3: 27590979 | 13437 | 3: 27591026 |
| 13438 | 3: 27591357 | 13439 | 3: 27591992 | 13440 | 3: 27592060 | 13441 | 3: 27592156 | 13442 | 3: 27592425 |
| 13443 | 3: 27592963 | 13447 | 3: 27618230 | 13448 | 3: 27623256 | 13449 | 3: 27624318 | 13450 | 3: 27624378 |
| 13451 | 3: 27626096 | 13488 | 3: 28582812 | 13489 | 3: 28655869 | 13493 | 3: 28699418 | 13494 | 3: 28701817 |
| 13496 | 3: 28737754 | 13497 | 3: 28739308 | 13502 | 3: 28795698 | 13503 | 3: 28847511 | 13512 | 3: 29091766 |
| 13517 | 3: 29129658 | 13528 | 3: 29280162 | 13530 | 3: 29285000 | 13543 | 3: 29574496 | 13776 | 3: 33446479 |
| 13782 | 3: 33558867 | 13874 | 3: 35055289 | 13935 | 3: 36314410 | 13938 | 3: 36323470 | 13942 | 3: 36436943 |
| 13943 | 3: 36535092 | 13955 | 3: 36838325 | 13956 | 3: 36846776 | 13971 | 3: 37286638 | 14025 | 3: 39287939 |
| 14031 | 3: 39484220 | 14091 | 3: 41318467 | 14096 | 3: 41450661 | 14106 | 3: 41608150 | 14116 | 3: 41941631 |
| 14121 | 3: 42042149 | 14131 | 3: 42370072 | 14173 | 3: 43226312 | 14205 | 3: 43968451 | 14208 | 3: 44136086 |
| 14252 | 3: 45371773 | 14253 | 3: 45389341 | 14259 | 3: 45661559 | 14287 | 3: 46609755 | 14319 | 3: 47528437 |
| 14325 | 3: 47614770 | 14335 | 3: 47862102 | 14340 | 3: 47901200 | 14346 | 3: 48004343 | 14371 | 3: 48662558 |
| 14376 | 3: 48794169 | 14383 | 3: 48908474 | 14388 | 3: 48945044 | 14391 | 3: 48996518 | 14393 | 3: 49005907 |
| 14397 | 3: 49066263 | 14400 | 3: 49170559 | 14411 | 3: 49437426 | 14430 | 3: 49640985 | 14431 | 3: 49686014 |
| 14432 | 3: 49688113 | 14433 | 3: 49693943 | 14434 | 3: 49694205 | 14435 | 3: 49694911 | 14436 | 3: 49695537 |
| 14437 | 3: 49695765 | 14438 | 3: 49695863 | 14439 | 3: 49695894 | 14440 | 3: 49696034 | 14441 | 3: 49697009 |
| 14442 | 3: 49697975 | 14443 | 3: 49698099 | 14444 | 3: 49698127 | 14445 | 3: 49698364 | 14446 | 3: 49698389 |
| 14447 | 3: 49698398 | 14448 | 3: 49698433 | 14449 | 3: 49698581 | 14450 | 3: 49698599 | 14451 | 3: 49698610 |
| 14452 | 3: 49698697 | 14453 | 3: 49698897 | 14454 | 3: 49698954 | 14455 | 3: 49698961 | 14456 | 3: 49699145 |
| 14457 | 3: 49699438 | 14458 | 3: 49699942 | 14459 | 3: 49700029 | 14460 | 3: 49700320 | 14461 | 3: 49700692 |
| 14462 | 3: 49700695 | 14463 | 3: 49700919 | 14464 | 3: 49701129 | 14465 | 3: 49701149 | 14466 | 3: 49701225 |
| 14467 | 3: 49701410 | 14468 | 3: 49701470 | 14469 | 3: 49702278 | 14470 | 3: 49702301 | 14476 | 3: 49768343 |
| 14489 | 3: 50198427 | 14509 | 3: 50849745 | 14526 | 3: 51610373 | 14542 | 3: 51914443 | 14546 | 3: 51963753 |
| 14547 | 3: 52005921 | 14550 | 3: 52127559 | 14559 | 3: 52354237 | 14560 | 3: 52412518 | 14562 | 3: 52416849 |
| 14563 | 3: 52423182 | 14564 | 3: 52427169 | 14566 | 3: 52429848 | 14567 | 3: 52433914 | 14570 | 3: 52487075 |
| 14577 | 3: 52690753 | 14580 | 3: 52745287 | 14583 | 3: 52852927 | 14591 | 3: 53113743 | 14592 | 3: 53117054 |
| 14593 | 3: 53126092 | 14594 | 3: 53126314 | 14595 | 3: 53126332 | 14597 | 3: 53143352 | 14600 | 3: 53244194 |
| 14608 | 3: 53474847 | 14610 | 3: 53481481 | 14611 | 3: 53510393 | 14652 | 3: 55080090 | 14670 | 3: 55424410 |
| 14740 | 3: 57665967 | 14760 | 3: 58213744 | 14761 | 3: 58216986 | 14771 | 3: 58501632 | 14789 | 3: 58991070 |
| 14855 | 3: 60699483 | 14856 | 3: 60699729 | 14857 | 3: 60704807 | 14871 | 3: 60976891 | 14874 | 3: 61002539 |
| 14876 | 3: 61050627 | 14911 | 3: 62289881 | 14936 | 3: 62942816 | 14979 | 3: 64119969 | 14994 | 3: 64762588 |
| 15003 | 3: 65001585 | 15011 | 3: 65190175 | 15045 | 3: 66062653 | 15056 | 3: 66307909 | 15064 | 3: 66462222 |
| 15108 | 3: 67467438 | 15119 | 3: 67681424 | 15160 | 3: 68627230 | 15190 | 3: 69504184 | 15196 | 3: 69782248 |
| 15197 | 3: 69782256 | 15198 | 3: 69782262 | 15199 | 3: 69786350 | 15215 | 3: 70251128 | 15260 | 3: 71848961 |
| 15262 | 3: 71934380 | 15307 | 3: 73666374 | 15325 | 3: 74232466 | 15331 | 3: 74366602 | 15394 | 3: 76166365 |
| 15408 | 3: 76630621 | 15458 | 3: 78060366 | 15471 | 3: 78544662 | 15539 | 3: 80535834 | 15585 | 3: 81521406 |
| 15586 | 3: 81525295 | 15639 | 3: 82930218 | 15642 | 3: 83051313 | 15674 | 3: 84083709 | 15679 | 3: 84152801 |
| 15685 | 3: 84286878 | 15713 | 3: 84688663 | 15738 | 3: 84955518 | 15745 | 3: 85050052 | 15746 | 3: 85066223 |
| 15766 | 3: 85749188 | 15769 | 3: 85769939 | 15774 | 3: 85846088 | 15776 | 3: 85897724 | 15784 | 3: 85979828 |
| 15785 | 3: 86001057 | 15786 | 3: 86027116 | 15846 | 3: 87314340 | 15854 | 3: 87568457 | 15855 | 3: 87570687 |
| 15857 | 3: 87576354 | 15858 | 3: 87576887 | 15859 | 3: 87596560 | 15865 | 3: 87888225 | 15900 | 3: 88530363 |
| 15903 | 3: 88603888 | 15905 | 3: 88620871 | 15926 | 3: 88972091 | 15934 | 3: 89181812 | 15938 | 3: 89279883 |
| 15955 | 3: 89599405 | 15989 | 3: 90306732 | 15990 | 3: 90333561 | 16033 | 3: 91219484 | 16036 | 3: 91278796 |
| 16051 | 3: 91695764 | 16084 | 3: 92543467 | 1610 | 3: 92905285 | 16113 | 3: 93070586 | 16148 | 3: 93889901 |
| 16213 | 3: 95744017 | 16218 | 3: 95893190 | 16222 | 3: 96032684 | 16228 | 3: 96151926 | 16281 | 3: 97566938 |
| 16299 | 3: 97901912 | 16346 | 3: 99237470 | 16360 | 3: 99532212 | 16362 | 3: 99579446 | 16424 | 3: 100686242 |
| 16429 | 3: 100725968 | 16463 | 3: 101430182 | 16476 | 3: 101718735 | 16488 | 3: 102039758 | 16507 | 3: 102478118 |
| 16508 | 3: 102487038 | 16525 | 3: 102814655 | 16545 | 3: 103051617 | 16547 | 3: 103073339 | 16685 | 3: 105603357 |
| 16698 | 3: 105784125 | 16760 | 3: 106768484 | 16763 | 3: 106821154 | 16796 | 3: 107517305 | 16797 | 3: 107520457 |
| 16805 | 3: 107754888 | 16842 | 3: 108517017 | 16865 | 3: 108844131 | 16904 | 3: 109724431 | 16905 | 3: 109725745 |
| 16906 | 3: 109730150 | 16907 | 3: 109731953 | 16908 | 3: 109737608 | 16909 | 3: 109737977 | 16910 | 3: 109738917 |
| 16980 | 3: 111463594 | 16984 | 3: 111515807 | 17006 | 3: 111954834 | 17039 | 3: 112612031 | 17040 | 3: 112625613 |
| 17055 | 3: 112990876 | 17088 | 3: 113561927 | 17123 | 3: 114126601 | 17131 | 3: 114256669 | 17145 | 3: 114503215 |
| 17147 | 3: 114519254 | 17151 | 3: 114571821 | 17153 | 3: 114600016 | 17154 | 3: 114600017 | 17161 | 3: 114722650 |
| 17203 | 3: 115249589 | 17205 | 3: 115271507 | 17250 | 3: 116030538 | 17298 | 3: 116861627 | 17300 | 3: 116869634 |
| 17301 | 3: 116871170 | 17303 | 3: 116878333 | 17305 | 3: 116901187 | 17306 | 3: 116901527 | 17307 | 3: 116903799 |
| 17311 | 3: 116932009 | 17340 | 3: 117275171 | 17356 | 3: 117642247 | 17357 | 3: 117642545 | 17358 | 3: 117643811 |
| 17359 | 3: 117644307 | 17398 | 3: 118174177 | 17427 | 3: 118632548 | 17445 | 3: 118859735 | 17446 | 3: 118864123 |
| 17451 | 3: 118890670 | 17500 | 3: 120280786 | 17594 | 4: 2379436 | 17634 | 4: 3851682 | 17635 | 4: 3859753 |
| 17638 | 4: 3982830 | 17645 | 4: 4155060 | 17665 | 4: 4566566 | 17667 | 4: 4584480 | 17668 | 4: 4589135 |
| 17669 | 4: 4591392 | 17670 | 4: 4591448 | 17677 | 4: 4684194 | 17681 | 4: 4780212 | 17682 | 4: 4780800 |
| 17683 | 4: 4786841 | 17687 | 4: 4824857 | 17690 | 4: 4865949 | 17694 | 4: 4969955 | 17719 | 4: 5368040 |
| 17727 | 4: 5444338 | 17738 | 4: 5704758 | 17765 | 4: 6383685 | 17791 | 4: 7064916 | 17803 | 4: 7319609 |
| 17805 | 4: 7325326 | 17815 | 4: 7454936 | 17830 | 4: 7796044 | 17846 | 4: 8193002 | 17869 | 4: 8790081 |
| 17881 | 4: 9092344 | 17895 | 4: 9372959 | 17903 | 4: 9479800 | 17917 | 4: 9747477 | 17933 | 4: 9983918 |
| 17934 | 4: 9983950 | 17935 | 4: 9984121 | 17936 | 4: 9984185 | 17937 | 4: 9984248 | 17938 | 4: 9984598 |
| 17940 | 4: 9986515 | 17971 | 4: 10645340 | 17974 | 4: 10692378 | 17979 | 4: 10807167 | 17980 | 4: 10817042 |
| 17982 | 4: 10817118 | 18028 | 4: 11882269 | 18081 | 4: 13277909 | 18083 | 4: 13314057 | 18084 | 4: 13315343 |
| 18132 | 4: 14870332 | 18185 | 4: 16314407 | 18193 | 4: 16510166 | 18212 | 4: 17151245 | 18220 | 4: 17227279 |
| 18227 | 4: 17429209 | 18244 | 4: 17664281 | 18251 | 4: 17934274 | 18292 | 4: 18879751 | 18295 | 4: 18927680 |
| 18301 | 4: 18998283 | 18302 | 4: 18999825 | 18303 | 4: 19004726 | 18304 | 4: 19005718 | 18305 | 4: 19006333 |
| 18306 | 4: 19007854 | 18307 | 4: 19007876 | 18308 | 4: 19008526 | 18309 | 4: 19008760 | 18310 | 4: 19010468 |
| 18331 | 4: 19684498 | 18332 | 4: 19687001 | 18333 | 4: 19687703 | 18334 | 4: 19688039 | 18335 | 4: 19690469 |
| 18336 | 4: 19690711 | 18337 | 4: 19692144 | 18338 | 4: 19692593 | 18339 | 4: 19692676 | 18340 | 4: 19693098 |
| 18341 | 4: 19693119 | 18342 | 4: 19694330 | 18343 | 4: 19694937 | 18344 | 4: 19695576 | 18345 | 4: 19695681 |
| 18346 | 4: 19696720 | 18347 | 4: 19696767 | 18348 | 4: 19697089 | 18349 | 4: 19697301 | 18350 | 4: 19697430 |
| 18351 | 4: 19697544 | 18352 | 4: 19697663 | 18354 | 4: 19748494 | 18359 | 4: 19938692 | 18361 | 4: 19989444 |
| 18375 | 4: 20261489 | 18376 | 4: 20265073 | 18392 | 4: 20703476 | 18401 | 4: 20822864 | 18421 | 4: 21167294 |
| 18431 | 4: 21342092 | 18432 | 4: 21358199 | 18440 | 4: 21699344 | 18457 | 4: 22138635 | 18466 | 4: 22471078 |
| 18468 | 4: 22523960 | 18481 | 4: 22728193 | 18490 | 4: 22923982 | 18497 | 4: 23121119 | 18500 | 4: 23196768 |
| 18504 | 4: 23389777 | 18530 | 4: 24084837 | 18546 | 4: 24614792 | 18563 | 4: 25092475 | 18600 | 4: 26062712 |
| 18625 | 4: 26743642 | 18628 | 4: 26796377 | 18632 | 4: 26851816 | 18643 | 4: 27158677 | 18656 | 4: 27519461 |
| 18679 | 4: 28114813 | 18697 | 4: 28590705 | 18717 | 4: 29227466 | 18733 | 4: 29791373 | 18759 | 4: 30557284 |
| 18768 | 4: 30768290 | 18782 | 4: 31109031 | 18806 | 4: 31790626 | 18808 | 4: 31802602 | 18827 | 4: 32352959 |
| 18838 | 4: 32687897 | 18870 | 4: 33603343 | 18902 | 4: 34379775 | 18952 | 4: 36053918 | 18953 | 4: 36057890 |
| 18966 | 4: 36473548 | 18993 | 4: 37223623 | 19001 | 4: 37381316 | 19019 | 4: 38054717 | 19024 | 4: 38091098 |
| 19030 | 4: 38294871 | 19041 | 4: 38579078 | 19042 | 4: 38591331 | 19060 | 4: 39231026 | 19066 | 4: 39400801 |
| 19084 | 4: 39970721 | 19114 | 4: 40595576 | 19133 | 4: 40929985 | 19178 | 4: 42197758 | 19207 | 4: 43094045 |
| 19209 | 4: 43138220 | 19236 | 4: 43793806 | 19294 | 4: 45527845 | 19336 | 4: 46745755 | 19366 | 4: 47300099 |
| 19380 | 4: 47488474 | 19388 | 4: 47696820 | 19425 | 4: 49010238 | 19454 | 4: 49228470 | 19465 | 4: 49442617 |
| 19505 | 4: 50866615 | 19529 | 4: 51642850 | 19540 | 4: 52014153 | 19574 | 4: 53063969 | 19623 | 4: 54533598 |
| 19654 | 4: 55652059 | 19655 | 4: 55666248 | 19666 | 4: 55979945 | 19678 | 4: 56152616 | 19746 | 4: 58369087 |
| 19753 | 4: 58591231 | 19754 | 4: 58596005 | 19769 | 4: 59119437 | 19775 | 4: 59214261 | 19791 | 4: 59626845 |
| 19794 | 4: 59754417 | 19815 | 4: 60395032 | 19869 | 4: 61855772 | 19915 | 4: 63120245 | 19920 | 4: 63231993 |
| 19939 | 4: 63706634 | 19941 | 4: 63737742 | 19965 | 4: 64527362 | 19979 | 4: 64927929 | 19981 | 4: 64973879 |
| 20073 | 4: 67329757 | 20080 | 4: 67492078 | 20083 | 4: 67548081 | 20121 | 4: 68635250 | 20122 | 4: 68672213 |
| 20195 | 4: 70609606 | 20233 | 4: 71767720 | 20235 | 4: 71789741 | 20358 | 4: 75159207 | 20362 | 4: 75242210 |
| 20385 | 4: 75797380 | 20413 | 4: 76318250 | 20436 | 4: 77029452 | 20481 | 4: 77948146 | 20491 | 4: 78185575 |
| 20539 | 4: 79657303 | 20543 | 4: 79839208 | 20568 | 4: 80186925 | 20573 | 4: 80245076 | 20587 | 4: 80584868 |
| 20592 | 4: 80719912 | 20661 | 4: 82586451 | 20666 | 4: 82698761 | 20712 | 4: 83936797 | 20729 | 4: 84451586 |
| 20765 | 4: 85525303 | 20774 | 4: 85682095 | 20781 | 4: 85880701 | 20796 | 4: 86159050 | 20846 | 4: 87474051 |
| 20850 | 4: 87586277 | 20873 | 4: 88094522 | 20894 | 4: 88685340 | 20914 | 4: 89467898 | 20924 | 4: 89712815 |
| 20974 | 4: 90944945 | 21037 | 4: 92282682 | 21054 | 4: 92473823 | 21057 | 4: 92523126 | 21063 | 4: 92640436 |
| 21103 | 4: 93420321 | 21105 | 4: 93454704 | 21113 | 4: 93574942 | 21118 | 4: 93658997 | 21176 | 4: 95336071 |
| 21177 | 4: 95348378 | 21181 | 4: 95493235 | 21227 | 4: 96019036 | 21262 | 4: 96809210 | 21279 | 4: 96825379 |
| 21360 | 4: 96942694 | 21366 | 4: 97025110 | 21436 | 4: 97663817 | 21456 | 4: 98220002 | 21526 | 4: 100120727 |
| 21537 | 4: 100380798 | 21545 | 4: 100648109 | 21550 | 4: 100737518 | 21555 | 4: 100801331 | 21644 | 4: 102776227 |
| 21654 | 4: 103076079 | 21677 | 4: 103575595 | 21680 | 4: 103670470 | 21746 | 4: 105364910 | 21757 | 4: 105516749 |
| 21761 | 4: 105568960 | 21777 | 4: 106062418 | 21819 | 4: 106715919 | 21888 | 4: 107648399 | 21906 | 4: 108309499 |
| 21907 | 4: 108319723 | 21926 | 4: 108947713 | 21940 | 4: 109363070 | 21947 | 4: 109525615 | 21948 | 4: 109528067 |
| 21949 | 4: 109528137 | 21950 | 4: 109529095 | 21951 | 4: 109529531 | 21952 | 4: 109529976 | 21953 | 4: 109530257 |
| 21954 | 4: 109530338 | 21955 | 4: 109532512 | 21956 | 4: 109533036 | 21957 | 4: 109538711 | 21958 | 4: 109538973 |
| 21959 | 4: 109545654 | 21961 | 4: 109576187 | 21985 | 4: 110235765 | 21986 | 4: 110257114 | 21999 | 4: 110637707 |
| 22000 | 4: 110672104 | 22001 | 4: 110703849 | 22014 | 4: 110890327 | 22025 | 4: 111120368 | 22026 | 4: 111120557 |
| 22061 | 4: 111707261 | 22062 | 4: 111708263 | 22063 | 4: 111711546 | 22064 | 4: 111711848 | 22065 | 4: 111713444 |
| 22086 | 4: 112129918 | 22089 | 4: 112149705 | 22095 | 4: 112210325 | 22107 | 4: 112369141 | 22119 | 4: 112555531 |
| 22179 | 4: 113489475 | 22192 | 4: 113598124 | 22193 | 4: 113600602 | 22194 | 4: 113601769 | 22196 | 4: 113610307 |
| 22197 | 4: 113612170 | 22198 | 4: 113614202 | 22202 | 4: 113636177 | 22203 | 4: 113637533 | 22204 | 4: 113639510 |
| 22205 | 4: 113645913 | 22206 | 4: 113646772 | 22207 | 4: 113647228 | 22208 | 4: 113647637 | 22209 | 4: 113649086 |
| 22210 | 4: 113650751 | 22211 | 4: 113653050 | 22212 | 4: 113670640 | 22213 | 4: 113670882 | 22214 | 4: 113671866 |
| 22215 | 4: 113673914 | 22216 | 4: 113673955 | 22217 | 4: 113676621 | 22218 | 4: 113680315 | 22351 | 4: 115923041 |
| 22368 | 4: 116278293 | 22377 | 4: 116438965 | 22393 | 4: 116746277 | 22401 | 4: 116897937 | 22404 | 4: 116926240 |
| 22405 | 4: 116961074 | 22514 | 4: 119167733 | 22520 | 4: 119293731 | 22547 | 5: 35727 | 22548 | 5: 42965 |
| 22549 | 5: 46543 | 22550 | 5: 56066 | 22551 | 5: 57672 | 22554 | 5: 90000 | 22555 | 5: 93744 |
| 22556 | 5: 95203 | 22557 | 5: 102783 | 22558 | 5: 126748 | 22559 | 5: 149452 | 22560 | 5: 156011 |
| 22562 | 5: 157715 | 22563 | 5: 159858 | 22578 | 5: 818560 | 22609 | 5: 1354890 | 22616 | 5: 1388630 |
| 22619 | 5: 1458836 | 22642 | 5: 1946239 | 22703 | 5: 3492443 | 22727 | 5: 4322265 | 22743 | 5: 4570191 |
| 22752 | 5: 4731333 | 22754 | 5: 4732613 | 22775 | 5: 5220278 | 22799 | 5: 5717840 | 22851 | 5: 6927428 |
| 22853 | 5: 6949366 | 22892 | 5: 7781205 | 22896 | 5: 7895175 | 22903 | 5: 8176883 | 22949 | 5: 9380271 |
| 22955 | 5: 9551052 | 22956 | 5: 9555919 | 22960 | 5: 9636042 | 22996 | 5: 10313930 | 23013 | 5: 10668196 |
| 23026 | 5: 10924954 | 23029 | 5: 10994036 | 23065 | 5: 12039811 | 23100 | 5: 12501985 | 23102 | 5: 12524034 |
| 23125 | 5: 13140668 | 23167 | 5: 14461980 | 23181 | 5: 14793206 | 23214 | 5: 15447067 | 23218 | 5: 15558924 |
| 23257 | 5: 16336560 | 23268 | 5: 16719670 | 23269 | 5: 16739498 | 23279 | 5: 17077362 | 23280 | 5: 17078096 |
| 23292 | 5: 17322229 | 23296 | 5: 17492634 | 23298 | 5: 17500602 | 23301 | 5: 17665160 | 23307 | 5: 17903579 |
| 23313 | 5: 18155232 | 23326 | 5: 18518099 | 23364 | 5: 19695581 | 23400 | 5: 20738200 | 23430 | 5: 21391973 |
| 23432 | 5: 21432976 | 23439 | 5: 21643782 | 23440 | 5: 21648427 | 23451 | 5: 21871823 | 23459 | 5: 21991889 |
| 23484 | 5: 22479526 | 23485 | 5: 22487130 | 23543 | 5: 23622599 | 23555 | 5: 23928759 | 23558 | 5: 23956744 |
| 23613 | 5: 25183734 | 23665 | 5: 26532385 | 23677 | 5: 26846005 | 23680 | 5: 26972277 | 23681 | 5: 26972772 |
| 23690 | 5: 27127258 | 23702 | 5: 27439061 | 23707 | 5: 27544684 | 23733 | 5: 27949133 | 23757 | 5: 28498202 |
| 23799 | 5: 29557818 | 23812 | 5: 29809074 | 23822 | 5: 29994317 | 23825 | 5: 30019258 | 23946 | 5: 33177185 |
| 23952 | 5: 33331535 | 23953 | 5: 33336405 | 23954 | 5: 33337110 | 23968 | 5: 33738237 | 24007 | 5: 34880473 |
| 24042 | 5: 35923865 | 24045 | 5: 36012922 | 24048 | 5: 36038265 | 24049 | 5: 36042606 | 24058 | 5: 36409810 |
| 24074 | 5: 36939207 | 24120 | 5: 37996697 | 24182 | 5: 39657770 | 24190 | 5: 39821210 | 24193 | 5: 39927222 |
| 24238 | 5: 41211155 | 24240 | 5: 41232876 | 24241 | 5: 41235105 | 24242 | 5: 41236186 | 24258 | 5: 41776747 |
| 24279 | 5: 42522952 | 24322 | 5: 43563557 | 24327 | 5: 43634786 | 24366 | 5: 44572552 | 24385 | 5: 45181510 |
| 24386 | 5: 45181566 | 24402 | 5: 45615706 | 24417 | 5: 45973787 | 24431 | 5: 46166472 | 24441 | 5: 46464570 |
| 24443 | 5: 46517477 | 24452 | 5: 46782312 | 24458 | 5: 46936034 | 24459 | 5: 46938241 | 24460 | 5: 46938449 |
| 24461 | 5: 46940754 | 24462 | 5: 46941677 | 24475 | 5: 47204347 | 24522 | 5: 48389782 | 24529 | 5: 48667259 |
| 24535 | 5: 48760782 | 24540 | 5: 48866791 | 24550 | 5: 49202198 | 24559 | 5: 49562957 | 24562 | 5: 49625601 |
| 24570 | 5: 49842789 | 24607 | 5: 50836826 | 24779 | 5: 55323383 | 24806 | 5: 55945914 | 24858 | 5: 57094271 |
| 24893 | 5: 58015125 | 24920 | 5: 59115316 | 24926 | 5: 59337375 | 24942 | 5: 59884753 | 24948 | 5: 60008612 |
| 24991 | 5: 60879895 | 24994 | 5: 60984488 | 24999 | 5: 61164840 | 25018 | 5: 61884469 | 25034 | 5: 62444037 |
| 25035 | 5: 62445409 | 25036 | 5: 62449226 | 25037 | 5: 62449848 | 25038 | 5: 62450932 | 25055 | 5: 63033268 |
| 25082 | 5: 63536637 | 25111 | 5: 64402956 | 25124 | 5: 64829651 | 25128 | 5: 64926399 | 25129 | 5: 64926523 |
| 25130 | 5: 64928264 | 25164 | 5: 65659261 | 25171 | 5: 65755099 | 25197 | 5: 66595784 | 25208 | 5: 66799811 |
| 25219 | 5: 67271370 | 25335 | 5: 69618545 | 25426 | 5: 71726794 | 25464 | 5: 72378602 | 25466 | 5: 72402551 |
| 25482 | 5: 73010927 | 25486 | 5: 73094311 | 25488 | 5: 73189660 | 25550 | 5: 74746217 | 25597 | 5: 75667991 |
| 25606 | 5: 75883986 | 25611 | 5: 75946414 | 25665 | 5: 77347106 | 25680 | 5: 77945857 | 25725 | 5: 78973946 |
| 25727 | 5: 79017627 | 25735 | 5: 79334960 | 25748 | 5: 79714596 | 25749 | 5: 79722250 | 25781 | 5: 80877376 |
| 25791 | 5: 81278052 | 25871 | 5: 82792399 | 25884 | 5: 83174887 | 25891 | 5: 83348374 | 25900 | 5: 83570417 |
| 25915 | 5: 83863121 | 25918 | 5: 83916047 | 25923 | 5: 84010266 | 25926 | 5: 84064602 | 25927 | 5: 84064623 |
| 25928 | 5: 84064772 | 25929 | 5: 84065038 | 25930 | 5: 84065171 | 25931 | 5: 84065332 | 25932 | 5: 84065339 |
| 25933 | 5: 84065355 | 25934 | 5: 84066088 | 25935 | 5: 84066287 | 25936 | 5: 84067645 | 25937 | 5: 84068297 |
| 25938 | 5: 84068606 | 25939 | 5: 84068687 | 25940 | 5: 84068732 | 25941 | 5: 84068756 | 25942 | 5: 84070079 |
| 25943 | 5: 84070128 | 25944 | 5: 84076319 | 25945 | 5: 84076527 | 25946 | 5: 84077100 | 25947 | 5: 84080168 |
| 25949 | 5: 84086306 | 25950 | 5: 84086861 | 25951 | 5: 84090372 | 25952 | 5: 84090487 | 25953 | 5: 84097284 |
| 25954 | 5: 84097885 | 25955 | 5: 84097985 | 25956 | 5: 84099214 | 25957 | 5: 84100319 | 25958 | 5: 84101337 |
| 25959 | 5: 84101438 | 25960 | 5: 84101462 | 25961 | 5: 84101494 | 25962 | 5: 84101519 | 25963 | 5: 84101555 |
| 25964 | 5: 84101619 | 25965 | 5: 84101701 | 25966 | 5: 84102206 | 25967 | 5: 84102236 | 25969 | 5: 84103792 |
| 25970 | 5: 84103930 | 25971 | 5: 84104200 | 25972 | 5: 84107013 | 25974 | 5: 84115513 | 25975 | 5: 84115550 |
| 25976 | 5: 84115605 | 25977 | 5: 84115682 | 25978 | 5: 84115698 | 25979 | 5: 84115763 | 25980 | 5: 84115908 |
| 25981 | 5: 84116028 | 25982 | 5: 84116506 | 25983 | 5: 84116583 | 25984 | 5: 84119378 | 25985 | 5: 84119408 |
| 25986 | 5: 84119435 | 25987 | 5: 84120471 | 25988 | 5: 84120477 | 25989 | 5: 84120540 | 25990 | 5: 84120562 |
| 25991 | 5: 84120612 | 25992 | 5: 84120761 | 25993 | 5: 84120772 | 25994 | 5: 84120800 | 25995 | 5: 84120828 |
| 25996 | 5: 84120900 | 25997 | 5: 84120980 | 25998 | 5: 84120987 | 25999 | 5: 84121087 | 26000 | 5: 84121318 |
| 26001 | 5: 84122651 | 26002 | 5: 84122709 | 26003 | 5: 84122905 | 26004 | 5: 84123385 | 26005 | 5: 84123428 |
| 26006 | 5: 84124508 | 26007 | 5: 84124539 | 26008 | 5: 84124557 | 26009 | 5: 84124558 | 26010 | 5: 84124682 |
| 26011 | 5: 84125648 | 26012 | 5: 84125855 | 26013 | 5: 84126181 | 26014 | 5: 84126183 | 26015 | 5: 84126222 |
| 26016 | 5: 84126766 | 26017 | 5: 84126941 | 26018 | 5: 84126965 | 26019 | 5: 84126986 | 26020 | 5: 84127998 |
| 26021 | 5: 84128048 | 26022 | 5: 84128850 | 26023 | 5: 84128968 | 26024 | 5: 84129307 | 26025 | 5: 84129349 |
| 26026 | 5: 84129709 | 26027 | 5: 84130342 | 26028 | 5: 84130419 | 26029 | 5: 84130836 | 26030 | 5: 84130988 |
| 26031 | 5: 84131404 | 26032 | 5: 84131699 | 26033 | 5: 84131703 | 26034 | 5: 84131742 | 26035 | 5: 84132713 |
| 26036 | 5: 84132801 | 26037 | 5: 84132954 | 26038 | 5: 84133129 | 26039 | 5: 84133144 | 26040 | 5: 84133170 |
| 26041 | 5: 84133302 | 26042 | 5: 84133384 | 26043 | 5: 84133400 | 26044 | 5: 84133418 | 26045 | 5: 84135047 |
| 26046 | 5: 84135179 | 26047 | 5: 84135274 | 26048 | 5: 84137923 | 26049 | 5: 84138207 | 26050 | 5: 84138277 |
| 26051 | 5: 84138403 | 26052 | 5: 84138617 | 26053 | 5: 84138641 | 26054 | 5: 84138927 | 26055 | 5: 84142947 |
| 26056 | 5: 84143312 | 26069 | 5: 84405330 | 26105 | 5: 85329574 | 26123 | 5: 85783695 | 26129 | 5: 85892084 |
| 26147 | 5: 86451423 | 26153 | 5: 86534299 | 26158 | 5: 86590201 | 26161 | 5: 86629956 | 26175 | 5: 86950590 |
| 26207 | 5: 87624956 | 26209 | 5: 87645403 | 26219 | 5: 87859562 | 26287 | 5: 88745049 | 26319 | 5: 89399078 |
| 26320 | 5: 89427836 | 26334 | 5: 89821006 | 26385 | 5: 91059021 | 26400 | 5: 91433996 | 26401 | 5: 91479219 |
| 26434 | 5: 92344711 | 26436 | 5: 92351975 | 26437 | 5: 92360410 | 26440 | 5: 92389962 | 26441 | 5: 92399584 |
| 26442 | 5: 92402496 | 26443 | 5: 92402509 | 26444 | 5: 92407767 | 26445 | 5: 92412423 | 26446 | 5: 92412426 |
| 26447 | 5: 92412918 | 26448 | 5: 92415826 | 26449 | 5: 92416355 | 26450 | 5: 92418513 | 26451 | 5: 92422512 |
| 26452 | 5: 92422802 | 26453 | 5: 92423893 | 26454 | 5: 92426805 | 26455 | 5: 92428903 | 26456 | 5: 92428912 |
| 26457 | 5: 92429850 | 26458 | 5: 92429875 | 26459 | 5: 92430956 | 26460 | 5: 92431470 | 26461 | 5: 92431717 |
| 26462 | 5: 92431719 | 26463 | 5: 92432015 | 26464 | 5: 92432232 | 26465 | 5: 92432425 | 26466 | 5: 92432633 |
| 26472 | 5: 92541180 | 26487 | 5: 92897747 | 26582 | 5: 95173664 | 26584 | 5: 95197902 | 26656 | 5: 96676782 |
| 26698 | 5: 97374091 | 26709 | 5: 97622463 | 26728 | 5: 98063850 | 26729 | 5: 98064276 | 26735 | 5: 98193113 |
| 26736 | 5: 98202197 | 26738 | 5: 98236533 | 26768 | 5: 98748721 | 26772 | 5: 98826571 | 26828 | 5: 99948490 |
| 26861 | 5: 100535488 | 26863 | 5: 100631069 | 26870 | 5: 100703214 | 26884 | 5: 100953490 | 26893 | 5: 101083268 |
| 26894 | 5: 101089292 | 26899 | 5: 101221558 | 26914 | 5: 101607756 | 26921 | 5: 101673726 | 26928 | 5: 101914630 |
| 26940 | 5: 102460767 | 26943 | 5: 102476964 | 26968 | 5: 103154349 | 26986 | 5: 103552417 | 27001 | 5: 103882706 |
| 27005 | 5: 103981305 | 27068 | 5: 105100005 | 27114 | 5: 105867389 | 27140 | 5: 106221085 | 27148 | 5: 106293076 |
| 27162 | 5: 106480592 | 27188 | 5: 106887255 | 27202 | 5: 107008747 | 27222 | 5: 107342588 | 27232 | 5: 107495491 |
| 27234 | 5: 107522927 | 27269 | 5: 108224683 | 27274 | 5: 108258258 | 27280 | 5: 108472906 | 27281 | 5: 108473260 |
| 27282 | 5: 108476147 | 27283 | 5: 108478132 | 27284 | 5: 108485933 | 27285 | 5: 108486228 | 27286 | 5: 108486756 |
| 27287 | 5: 108487832 | 27288 | 5: 108489677 | 27289 | 5: 108490693 | 27290 | 5: 108490734 | 27295 | 5: 108546887 |
| 27297 | 5: 108575310 | 27399 | 5: 110511118 | 27440 | 5: 111144555 | 27462 | 5: 111761777 | 27467 | 5: 111860816 |
| 27481 | 5: 112115946 | 27519 | 5: 112982896 | 27525 | 5: 113133130 | 27539 | 5: 113320727 | 27547 | 5: 113650431 |
| 27566 | 5: 114143008 | 27569 | 5: 114206160 | 27572 | 5: 114283857 | 27580 | 5: 114719162 | 27583 | 5: 114775926 |
| 27605 | 5: 115427295 | 27652 | 5: 116126971 | 27656 | 5: 116179833 | 27676 | 5: 116586289 | 27677 | 5: 116588965 |
| 27678 | 5: 116590488 | 27679 | 5: 116590506 | 27680 | 5: 116590745 | 27682 | 5: 116592015 | 27683 | 5: 116592619 |
| 27684 | 5: 116592903 | 27687 | 5: 116593192 | 27688 | 5: 116593569 | 27694 | 5: 116594398 | 27695 | 5: 116594541 |
| 27696 | 5: 116594546 | 27697 | 5: 116594548 | 27698 | 5: 116594847 | 27699 | 5: 116595244 | 27700 | 5: 116595727 |
| 27701 | 5: 116595799 | 27702 | 5: 116596186 | 27704 | 5: 116596441 | 27705 | 5: 116596625 | 27706 | 5: 116596745 |
| 27707 | 5: 116596913 | 27708 | 5: 116597118 | 27711 | 5: 116598702 | 27712 | 5: 116598835 | 27715 | 5: 116602527 |
| 27721 | 5: 116677042 | 27731 | 5: 116722878 | 27808 | 5: 117709009 | 27809 | 5: 117711361 | 27813 | 5: 117773986 |
| 27826 | 5: 117998759 | 27831 | 5: 118098871 | 27838 | 5: 118250217 | 27860 | 5: 118703768 | 27866 | 5: 118745750 |
| 27903 | 5: 119271970 | 27908 | 5: 119361103 | 27916 | 5: 119587043 | 27948 | 6: 528320 | 27970 | 6: 993117 |
| 27999 | 6: 1950764 | 28000 | 6: 1951302 | 28001 | 6: 1951498 | 28002 | 6: 1951714 | 28003 | 6: 1951719 |
| 28004 | 6: 1951800 | 28005 | 6: 1951981 | 28006 | 6: 1952392 | 28007 | 6: 1955233 | 28008 | 6: 1955330 |
| 28009 | 6: 1955401 | 28010 | 6: 1955549 | 28011 | 6: 1955698 | 28012 | 6: 1956232 | 28013 | 6: 1956278 |
| 28014 | 6: 1957428 | 28015 | 6: 1957620 | 28016 | 6: 1957800 | 28017 | 6: 1957812 | 28018 | 6: 1957895 |
| 28019 | 6: 1958152 | 28020 | 6: 1962365 | 28021 | 6: 1963113 | 28022 | 6: 1963759 | 28023 | 6: 1963825 |
| 28024 | 6: 1963953 | 28026 | 6: 1973036 | 28027 | 6: 1974486 | 28028 | 6: 1974895 | 28061 | 6: 2801468 |
| 28063 | 6: 2860306 | 28064 | 6: 2862765 | 28072 | 6: 3067202 | 28074 | 6: 3073689 | 28075 | 6: 3084367 |
| 28129 | 6: 3786375 | 28155 | 6: 4418616 | 28168 | 6: 4782253 | 28170 | 6: 4824255 | 28221 | 6: 6133626 |
| 28238 | 6: 6309983 | 28278 | 6: 7061434 | 28286 | 6: 7250536 | 28293 | 6: 7394514 | 28319 | 6: 8317563 |
| 28336 | 6: 8929534 | 28337 | 6: 8944681 | 28339 | 6: 8955494 | 28341 | 6: 8960665 | 28343 | 6: 8972751 |
| 28345 | 6: 8980990 | 28346 | 6: 8985187 | 28347 | 6: 8989449 | 28352 | 6: 9079158 | 28353 | 6: 9081859 |
| 28354 | 6: 9085203 | 28356 | 6: 9094449 | 28373 | 6: 9291690 | 28377 | 6: 9465992 | 28389 | 6: 9964731 |
| 28390 | 6: 9965161 | 28391 | 6: 9976901 | 28393 | 6: 9992803 | 28395 | 6: 10029462 | 28409 | 6: 10453731 |
| 28422 | 6: 10849905 | 28435 | 6: 11149738 | 28440 | 6: 11315175 | 28605 | 6: 15051875 | 28641 | 6: 15680456 |
| 28686 | 6: 16603866 | 28718 | 6: 17241712 | 28757 | 6: 18035615 | 28758 | 6: 18035662 | 28759 | 6: 18035884 |
| 28760 | 6: 18036845 | 28766 | 6: 18057767 | 28795 | 6: 18806853 | 28819 | 6: 19264271 | 28820 | 6: 19264527 |
| 28852 | 6: 19965110 | 28878 | 6: 20517010 | 28888 | 6: 20729903 | 28944 | 6: 22097118 | 28970 | 6: 22554116 |
| 29044 | 6: 24253816 | 29057 | 6: 24617112 | 29061 | 6: 24750491 | 29069 | 6: 24940966 | 29114 | 6: 25994106 |
| 29115 | 6: 25995000 | 29123 | 6: 26081649 | 29151 | 6: 26895539 | 29159 | 6: 27119474 | 29179 | 6: 27632577 |
| 29182 | 6: 27765059 | 29183 | 6: 27794604 | 29196 | 6: 28156905 | 29204 | 6: 28392137 | 29233 | 6: 29155222 |
| 29374 | 6: 30901671 | 29490 | 6: 34238062 | 29722 | 6: 34572571 | 29939 | 6: 35180069 | 29996 | 6: 35924006 |
| 29997 | 6: 35924204 | 29998 | 6: 35925489 | 29999 | 6: 35931470 | 30000 | 6: 35932579 | 30010 | 6: 35978601 |
| 30133 | 6: 36426239 | 30136 | 6: 36429755 | 30138 | 6: 36430937 | 30160 | 6: 36467397 | 30164 | 6: 36475104 |
| 30165 | 6: 36477167 | 30168 | 6: 36481433 | 30169 | 6: 36481434 | 30171 | 6: 36483188 | 30174 | 6: 36489959 |
| 30175 | 6: 36490754 | 30234 | 6: 36545679 | 30259 | 6: 36568156 | 30261 | 6: 36568846 | 30328 | 6: 36807893 |
| 30329 | 6: 36808225 | 30459 | 6: 37029628 | 30462 | 6: 37032995 | 30468 | 6: 37035541 | 30566 | 6: 37095299 |
| 30653 | 6: 37151905 | 30655 | 6: 37152373 | 30679 | 6: 37160005 | 30735 | 6: 37172974 | 30736 | 6: 37173048 |
| 30748 | 6: 37175662 | 30750 | 6: 37175773 | 30757 | 6: 37176677 | 30772 | 6: 37181087 | 30781 | 6: 37182773 |
| 30787 | 6: 37183901 | 30792 | 6: 37189736 | 30794 | 6: 37192102 | 30802 | 6: 37214389 | 30852 | 6: 37251132 |
| 30858 | 6: 37262825 | 30921 | 6: 37299855 | 31027 | 6: 37329948 | 31093 | 6: 37365265 | 31180 | 6: 37431101 |
| 31284 | 6: 37636883 | 31285 | 6: 37636900 | 31524 | 6: 37936087 | 31846 | 6: 38545113 | 31894 | 6: 38558946 |
| 31930 | 6: 38800398 | 31931 | 6: 38830763 | 31935 | 6: 38855286 | 31936 | 6: 38862311 | 31937 | 6: 38862987 |
| 31938 | 6: 38875748 | 31940 | 6: 38879318 | 31941 | 6: 38882135 | 31980 | 6: 39565429 | 31983 | 6: 39593110 |
| 32001 | 6: 39758696 | 32010 | 6: 39853149 | 32018 | 6: 40036007 | 32020 | 6: 40036943 | 32022 | 6: 40040343 |
| 32023 | 6: 40053452 | 32025 | 6: 40058671 | 32030 | 6: 40130949 | 32056 | 6: 40611279 | 32063 | 6: 40707783 |
| 32064 | 6: 40708542 | 32065 | 6: 40708988 | 32066 | 6: 40709098 | 32067 | 6: 40709130 | 32068 | 6: 40709457 |
| 32069 | 6: 40709727 | 32070 | 6: 40709846 | 32071 | 6: 40711310 | 32072 | 6: 40711346 | 32073 | 6: 40711409 |
| 32074 | 6: 40711422 | 32075 | 6: 40711439 | 32076 | 6: 40713027 | 32077 | 6: 40713351 | 32078 | 6: 40713605 |
| 32079 | 6: 40713909 | 32080 | 6: 40714153 | 32081 | 6: 40714938 | 32082 | 6: 40715195 | 32083 | 6: 40716811 |
| 32084 | 6: 40721324 | 32087 | 6: 40743678 | 32090 | 6: 40770454 | 32091 | 6: 40771992 | 32092 | 6: 40771993 |
| 32186 | 6: 41827472 | 32240 | 6: 42833384 | 32249 | 6: 42951534 | 32250 | 6: 42951582 | 32251 | 6: 42951695 |
| 32252 | 6: 42951801 | 32253 | 6: 42952447 | 32254 | 6: 42953604 | 32255 | 6: 42954090 | 32256 | 6: 42956624 |
| 32257 | 6: 42957873 | 32258 | 6: 42961867 | 32259 | 6: 42963490 | 32260 | 6: 42966370 | 32261 | 6: 42969572 |
| 32265 | 6: 42996149 | 32268 | 6: 43024569 | 32269 | 6: 43035223 | 32270 | 6: 43035520 | 32278 | 6: 43178587 |
| 32292 | 6: 43450785 | 32322 | 6: 43878243 | 32328 | 6: 43959607 | 32364 | 6: 44686809 | 32417 | 6: 44828139 |
| 32510 | 6: 45452605 | 32511 | 6: 45453963 | 32521 | 6: 45702584 | 32555 | 6: 46424441 | 32556 | 6: 46446420 |
| 32564 | 6: 46615384 | 32573 | 6: 46883981 | 32575 | 6: 46926732 | 32584 | 6: 47108047 | 32611 | 6: 47785922 |
| 32645 | 6: 48798347 | 32726 | 6: 50704099 | 32732 | 6: 50885777 | 32742 | 6: 51188886 | 32764 | 6: 51982250 |
| 32769 | 6: 52083644 | 32804 | 6: 52427412 | 32805 | 6: 52431484 | 32806 | 6: 52434675 | 32807 | 6: 52444382 |
| 32808 | 6: 52444842 | 32809 | 6: 52451412 | 32819 | 6: 52675393 | 32832 | 6: 53029143 | 32838 | 6: 53151451 |
| 32907 | 6: 55106382 | 32908 | 6: 55121620 | 32912 | 6: 55288741 | 32916 | 6: 55337592 | 32921 | 6: 55478053 |
| 32922 | 6: 55478241 | 32923 | 6: 55480385 | 32924 | 6: 55481575 | 32925 | 6: 55483221 | 32926 | 6: 55483692 |
| 32927 | 6: 55484196 | 32928 | 6: 55484209 | 32929 | 6: 55484776 | 32930 | 6: 55484984 | 32931 | 6: 55484996 |
| 32932 | 6: 55485652 | 32933 | 6: 55487306 | 32934 | 6: 55487659 | 32935 | 6: 55487698 | 32936 | 6: 55487961 |
| 32937 | 6: 55488137 | 32938 | 6: 55488439 | 32939 | 6: 55498328 | 32945 | 6: 55534953 | 33154 | 6: 60793645 |
| 33178 | 6: 61401990 | 33201 | 6: 62075988 | 33220 | 6: 62746494 | 33287 | 6: 64826176 | 33288 | 6: 64860935 |
| 33301 | 6: 65368807 | 33320 | 6: 65902330 | 33385 | 6: 68144658 | 33560 | 6: 73271530 | 33568 | 6: 73524311 |
| 33587 | 6: 74274673 | 33588 | 6: 74278129 | 33590 | 6: 74325296 | 33595 | 6: 74538501 | 33604 | 6: 74934981 |
| 33638 | 6: 76140833 | 33664 | 6: 76838570 | 33669 | 6: 76918617 | 33761 | 6: 80397556 | 33786 | 6: 80984642 |
| 33856 | 6: 83284076 | 33857 | 6: 83290251 | 33889 | 6: 83810111 | 33919 | 6: 84577409 | 33945 | 6: 85170579 |
| 33958 | 6: 85452102 | 33960 | 6: 85473180 | 33973 | 6: 85798206 | 33990 | 6: 86146685 | 34015 | 6: 86736214 |
| 34039 | 6: 87076207 | 34076 | 6: 87751257 | 34095 | 6: 88150285 | 34146 | 6: 89563672 | 34160 | 6: 89750491 |
| 34167 | 6: 89906046 | 34223 | 6: 90999178 | 34252 | 6: 91356781 | 34256 | 6: 91397116 | 34311 | 6: 92717918 |
| 34325 | 6: 93027241 | 34355 | 6: 93561742 | 34379 | 6: 94081466 | 34380 | 6: 94087064 | 34382 | 6: 94089501 |
| 34383 | 6: 94094573 | 34384 | 6: 94096218 | 34385 | 6: 94098014 | 34386 | 6: 94098380 | 34387 | 6: 94098998 |
| 34456 | 6: 96190398 | 34499 | 6: 97061854 | 34533 | 6: 97481556 | 34534 | 6: 97481830 | 34535 | 6: 97482142 |
| 34536 | 6: 97486160 | 34588 | 6: 98499066 | 34597 | 6: 98688262 | 34647 | 6: 100180448 | 34648 | 6: 100181539 |
| 34649 | 6: 100181706 | 34650 | 6: 100181746 | 34651 | 6: 100182503 | 34652 | 6: 100183086 | 34653 | 6: 100183464 |
| 34654 | 6: 100185002 | 34655 | 6: 100185489 | 34656 | 6: 100185949 | 34657 | 6: 100186040 | 34658 | 6: 100186295 |
| 34673 | 6: 100520648 | 34698 | 6: 101088719 | 34699 | 6: 101089428 | 34765 | 6: 102387484 | 34775 | 6: 102527385 |
| 34820 | 6: 103206312 | 34874 | 6: 104209750 | 34913 | 6: 105016409 | 34983 | 6: 106924221 | 34985 | 6: 106930321 |
| 34998 | 6: 107232252 | 35017 | 6: 107833335 | 35031 | 6: 108314278 | 35032 | 6: 108316551 | 35035 | 6: 108336234 |
| 35040 | 6: 108483986 | 35074 | 6: 109282710 | 35086 | 6: 109549186 | 35112 | 6: 110036778 | 35120 | 6: 110267946 |
| 35121 | 6: 110296111 | 35154 | 6: 111027360 | 35170 | 6: 111251001 | 35181 | 6: 111506757 | 35194 | 6: 111752185 |
| 35195 | 6: 111753497 | 35196 | 6: 111755682 | 35203 | 6: 111845284 | 35215 | 6: 112274283 | 35225 | 6: 112517961 |
| 35254 | 6: 112951648 | 35258 | 6: 113193897 | 35264 | 6: 113278790 | 35269 | 6: 113338198 | 35302 | 6: 113832391 |
| 35304 | 6: 113899018 | 35316 | 6: 114099166 | 35317 | 6: 114104514 | 35338 | 6: 114493111 | 35356 | 6: 114802066 |
| 35377 | 6: 115232986 | 35397 | 6: 115802755 | 35522 | 7: 1173379 | 35548 | 7: 1688750 | 35608 | 7: 2637204 |
| 35647 | 7: 3292376 | 35723 | 7: 4848657 | 35745 | 7: 5160131 | 35746 | 7: 5169066 | 35754 | 7: 5443639 |
| 35777 | 7: 5627311 | 35779 | 7: 5637555 | 35814 | 7: 6109291 | 35815 | 7: 6109369 | 35816 | 7: 6110105 |
| 35817 | 7: 6110223 | 35818 | 7: 6110813 | 35819 | 7: 6112482 | 35820 | 7: 6112502 | 35821 | 7: 6112503 |
| 35822 | 7: 6112832 | 35824 | 7: 6114838 | 35825 | 7: 6118415 | 35826 | 7: 6118766 | 35827 | 7: 6119488 |
| 35828 | 7: 6122989 | 35829 | 7: 6123149 | 35830 | 7: 6123438 | 35831 | 7: 6128021 | 35832 | 7: 6130590 |
| 35833 | 7: 6130687 | 35834 | 7: 6131292 | 35835 | 7: 6131297 | 35836 | 7: 6132484 | 35837 | 7: 6132747 |
| 35838 | 7: 6133083 | 35840 | 7: 6137471 | 35854 | 7: 6339944 | 35855 | 7: 6341414 | 35856 | 7: 6341470 |
| 35857 | 7: 6341501 | 35858 | 7: 6345398 | 35860 | 7: 6348000 | 35861 | 7: 6348353 | 35862 | 7: 6352211 |
| 35863 | 7: 6356100 | 35864 | 7: 6356608 | 35865 | 7: 6356670 | 35881 | 7: 6710471 | 35894 | 7: 6824241 |
| 35903 | 7: 6999017 | 35914 | 7: 7143257 | 35929 | 7: 7557725 | 35930 | 7: 7562490 | 35933 | 7: 7584113 |
| 35959 | 7: 8549050 | 35966 | 7: 8914661 | 35977 | 7: 9686485 | 35978 | 7: 9760724 | 36006 | 7: 11067502 |
| 36013 | 7: 11123621 | 36031 | 7: 11430993 | 36036 | 7: 11668056 | 36046 | 7: 11952515 | 36081 | 7: 12499238 |
| 36157 | 7: 13717835 | 36185 | 7: 14182114 | 36243 | 7: 15057296 | 36303 | 7: 16284081 | 36325 | 7: 16641516 |
| 36363 | 7: 17636616 | 36365 | 7: 17641071 | 36384 | 7: 18214147 | 36409 | 7: 18665015 | 36416 | 7: 18849856 |
| 36433 | 7: 19227351 | 36442 | 7: 19317690 | 36444 | 7: 19333133 | 36461 | 7: 19721740 | 36516 | 7: 20334637 |
| 36524 | 7: 20395009 | 36536 | 7: 20701671 | 36537 | 7: 20704005 | 36539 | 7: 20747147 | 36548 | 7: 20935768 |
| 36639 | 7: 23007875 | 36650 | 7: 23297694 | 36651 | 7: 23350564 | 36657 | 7: 23528238 | 36658 | 7: 23528885 |
| 36684 | 7: 24067558 | 36692 | 7: 24236323 | 36709 | 7: 24746921 | 36734 | 7: 25342888 | 36737 | 7: 25403017 |
| 36843 | 7: 27517669 | 36845 | 7: 27577715 | 36846 | 7: 27588151 | 36892 | 7: 28685778 | 36910 | 7: 29075991 |
| 36945 | 7: 29836572 | 36952 | 7: 29934618 | 36955 | 7: 30045346 | 36969 | 7: 30374597 | 36979 | 7: 30662217 |
| 37046 | 7: 31780219 | 37055 | 7: 32039528 | 37090 | 7: 33006143 | 37103 | 7: 33314191 | 37140 | 7: 34167737 |
| 37157 | 7: 34561935 | 37166 | 7: 34800911 | 37204 | 7: 35829441 | 37224 | 7: 36375051 | 37225 | 7: 36381643 |
| 37271 | 7: 37512158 | 37358 | 7: 39431184 | 37383 | 7: 40018615 | 37386 | 7: 40105218 | 37387 | 7: 40106188 |
| 37403 | 7: 40313129 | 37421 | 7: 40543374 | 37501 | 7: 42717113 | 37502 | 7: 42793836 | 37562 | 7: 44383968 |
| 37577 | 7: 44807464 | 37579 | 7: 44874918 | 37589 | 7: 45086131 | 37597 | 7: 45264623 | 37598 | 7: 45268714 |
| 37606 | 7: 45462352 | 37628 | 7: 46197379 | 37629 | 7: 46207779 | 37647 | 7: 46698742 | 37652 | 7: 46763773 |
| 37665 | 7: 47007606 | 37666 | 7: 47009580 | 37667 | 7: 47010093 | 37668 | 7: 47010163 | 37669 | 7: 47011323 |
| 37670 | 7: 47011475 | 37671 | 7: 47012085 | 37672 | 7: 47012699 | 37674 | 7: 47017310 | 37675 | 7: 47018943 |
| 37676 | 7: 47021031 | 37716 | 7: 48370104 | 37736 | 7: 48785206 | 37758 | 7: 49338040 | 37807 | 7: 51678528 |
| 37832 | 7: 52217990 | 37892 | 7: 52801631 | 37901 | 7: 53051717 | 37903 | 7: 53072922 | 37907 | 7: 53192775 |
| 37929 | 7: 53916384 | 37998 | 7: 55634474 | 38004 | 7: 55875367 | 38018 | 7: 56132046 | 38036 | 7: 56553298 |
| 38044 | 7: 56873627 | 38072 | 7: 57843543 | 38089 | 7: 58267508 | 38091 | 7: 58338126 | 38139 | 7: 59331366 |
| 38146 | 7: 59368692 | 38166 | 7: 59579502 | 38249 | 7: 61303310 | 38261 | 7: 61485578 | 38264 | 7: 61501849 |
| 38305 | 7: 62532423 | 38306 | 7: 62554501 | 38309 | 7: 62558404 | 38377 | 7: 63949067 | 38388 | 7: 64285750 |
| 38397 | 7: 64625175 | 38412 | 7: 64930029 | 38413 | 7: 64937658 | 38420 | 7: 65072560 | 38432 | 7: 65258702 |
| 38507 | 7: 66460989 | 38538 | 7: 67323191 | 38555 | 7: 67781532 | 38557 | 7: 67828291 | 38563 | 7: 67940525 |
| 38586 | 7: 68625153 | 38589 | 7: 68641820 | 38642 | 7: 69866274 | 38644 | 7: 69879505 | 38646 | 7: 69905767 |
| 38653 | 7: 70037542 | 38655 | 7: 70093469 | 38656 | 7: 70099250 | 38666 | 7: 70331724 | 38669 | 7: 70419871 |
| 38689 | 7: 71130508 | 38698 | 7: 71415976 | 38716 | 7: 71712692 | 38717 | 7: 71727860 | 38722 | 7: 71825182 |
| 38723 | 7: 71830368 | 38725 | 7: 71921897 | 38754 | 7: 72753552 | 38794 | 7: 74007645 | 38826 | 7: 74850810 |
| 38869 | 7: 75797751 | 38879 | 7: 76006096 | 38880 | 7: 76006417 | 38881 | 7: 76010723 | 38916 | 7: 76940482 |
| 38930 | 7: 77150855 | 38941 | 7: 77453724 | 38950 | 7: 77656774 | 38964 | 7: 77949425 | 38989 | 7: 78379223 |
| 39000 | 7: 78748906 | 39008 | 7: 79016201 | 39009 | 7: 79016267 | 39010 | 7: 79016787 | 39011 | 7: 79017056 |
| 39012 | 7: 79017378 | 39013 | 7: 79017385 | 39014 | 7: 79017772 | 39015 | 7: 79017959 | 39016 | 7: 79018035 |
| 39017 | 7: 79018571 | 39018 | 7: 79018616 | 39019 | 7: 79018620 | 39020 | 7: 79019988 | 39021 | 7: 79023682 |
| 39022 | 7: 79025120 | 39023 | 7: 79025266 | 39024 | 7: 79041811 | 39025 | 7: 79042476 | 39027 | 7: 79048979 |
| 39028 | 7: 79070660 | 39029 | 7: 79071063 | 39031 | 7: 79135260 | 39032 | 7: 79135270 | 39040 | 7: 79367716 |
| 39056 | 7: 79660008 | 39076 | 7: 80098520 | 39077 | 7: 80104490 | 39096 | 7: 80463366 | 39111 | 7: 80573664 |
| 39122 | 7: 80835359 | 39133 | 7: 81097721 | 39221 | 7: 82912666 | 39241 | 7: 83502868 | 39259 | 7: 83929718 |
| 39299 | 7: 85098423 | 39310 | 7: 85314414 | 39315 | 7: 85586461 | 39383 | 7: 87496719 | 39413 | 7: 88462551 |
| 39425 | 7: 88705722 | 39622 | 7: 91895030 | 39706 | 7: 94524953 | 39712 | 7: 94671798 | 39713 | 7: 94672003 |
| 39714 | 7: 94688436 | 39715 | 7: 94691325 | 39728 | 7: 95018455 | 39768 | 7: 95975870 | 39773 | 7: 96088093 |
| 39774 | 7: 96093182 | 39795 | 7: 96175080 | 39796 | 7: 96175132 | 39797 | 7: 96175168 | 39798 | 7: 96175341 |
| 39799 | 7: 96175357 | 39800 | 7: 96175409 | 39801 | 7: 96175451 | 39802 | 7: 96175628 | 39803 | 7: 96176143 |
| 39804 | 7: 96176528 | 39805 | 7: 96176908 | 39809 | 7: 96193901 | 39812 | 7: 96199226 | 39815 | 7: 96205718 |
| 39816 | 7: 96205942 | 39817 | 7: 96206884 | 39818 | 7: 96207458 | 39819 | 7: 96207882 | 39820 | 7: 96208060 |
| 39821 | 7: 96208106 | 39822 | 7: 96208582 | 39823 | 7: 96208995 | 39824 | 7: 96209096 | 39825 | 7: 96209862 |
| 39826 | 7: 96211592 | 39827 | 7: 96211829 | 39828 | 7: 96216996 | 39832 | 7: 96221555 | 39833 | 7: 96223919 |
| 39834 | 7: 96228261 | 39835 | 7: 96228951 | 39836 | 7: 96231447 | 39837 | 7: 96235988 | 39838 | 7: 96236371 |
| 39839 | 7: 96236926 | 39840 | 7: 96239015 | 39841 | 7: 96239023 | 39842 | 7: 96242837 | 39843 | 7: 96246776 |
| 39844 | 7: 96246937 | 39845 | 7: 96248619 | 39846 | 7: 96248835 | 39847 | 7: 96249753 | 39848 | 7: 96249787 |
| 39849 | 7: 96249862 | 39850 | 7: 96254816 | 39851 | 7: 96263665 | 39852 | 7: 96264122 | 39853 | 7: 96264141 |
| 39854 | 7: 96265124 | 39855 | 7: 96265144 | 39856 | 7: 96265161 | 39857 | 7: 96265371 | 39858 | 7: 96265379 |
| 39860 | 7: 96271576 | 39861 | 7: 96271653 | 39862 | 7: 96273181 | 39863 | 7: 96273246 | 39864 | 7: 96273247 |
| 39865 | 7: 96273562 | 39866 | 7: 96273613 | 39867 | 7: 96274725 | 39869 | 7: 96291284 | 39870 | 7: 96291379 |
| 39871 | 7: 96291389 | 39872 | 7: 96291500 | 39873 | 7: 96291530 | 39874 | 7: 96293330 | 39875 | 7: 96293559 |
| 39876 | 7: 96293758 | 39877 | 7: 96293861 | 39878 | 7: 96295220 | 39879 | 7: 96295518 | 39880 | 7: 96295778 |
| 39881 | 7: 96295878 | 39882 | 7: 96295906 | 39883 | 7: 96295937 | 39884 | 7: 96296075 | 39885 | 7: 96296087 |
| 39886 | 7: 96296169 | 39887 | 7: 96296233 | 39888 | 7: 96296513 | 39889 | 7: 96296531 | 39890 | 7: 96296546 |
| 39891 | 7: 96296683 | 39892 | 7: 96297463 | 39893 | 7: 96297555 | 39894 | 7: 96297857 | 39895 | 7: 96298677 |
| 39896 | 7: 96299647 | 39897 | 7: 96300501 | 39898 | 7: 96303921 | 39899 | 7: 96304204 | 39900 | 7: 96305341 |
| 39901 | 7: 96305698 | 39902 | 7: 96307964 | 39903 | 7: 96308750 | 39904 | 7: 96308945 | 39905 | 7: 96309033 |
| 39906 | 7: 96310180 | 39907 | 7: 96311062 | 39909 | 7: 96314531 | 39910 | 7: 96314540 | 39913 | 7: 96318724 |
| 39914 | 7: 96322543 | 39915 | 7: 96322732 | 39916 | 7: 96323877 | 39917 | 7: 96323928 | 39918 | 7: 96324360 |
| 39919 | 7: 96330432 | 39920 | 7: 96331236 | 39921 | 7: 96331603 | 39922 | 7: 96333195 | 39923 | 7: 96333277 |
| 39924 | 7: 96333341 | 39925 | 7: 96333563 | 39926 | 7: 96333575 | 39927 | 7: 96334610 | 39928 | 7: 96334702 |
| 39929 | 7: 96334763 | 39930 | 7: 96335706 | 39931 | 7: 96337614 | 39933 | 7: 96354116 | 39935 | 7: 96355876 |
| 39936 | 7: 96358329 | 39937 | 7: 96361391 | 39938 | 7: 96363115 | 39940 | 7: 96374561 | 39949 | 7: 96519372 |
| 39953 | 7: 96606759 | 40022 | 7: 98399080 | 40043 | 7: 99244131 | 40046 | 7: 99254092 | 40085 | 7: 100105110 |
| 40106 | 7: 100563689 | 40108 | 7: 100578900 | 40112 | 7: 100659403 | 40127 | 7: 101215525 | 40166 | 7: 102357637 |
| 40201 | 7: 103052550 | 40216 | 7: 103329082 | 40224 | 7: 103487103 | 40229 | 7: 103675594 | 40232 | 7: 103729325 |
| 40239 | 7: 103982179 | 40240 | 7: 103982180 | 40241 | 7: 103982288 | 40242 | 7: 103982423 | 40243 | 7: 103985377 |
| 40253 | 7: 104230973 | 40291 | 7: 105373160 | 40294 | 7: 105399311 | 40296 | 7: 105442182 | 40297 | 7: 105515412 |
| 40324 | 7: 106146247 | 40372 | 7: 107094209 | 40415 | 7: 108495274 | 40496 | 8: 356371 | 40500 | 8: 395127 |
| 40585 | 8: 2113854 | 40598 | 8: 2387478 | 40600 | 8: 2420577 | 40637 | 8: 3508161 | 40640 | 8: 3527178 |
| 40645 | 8: 3640550 | 40647 | 8: 3666778 | 40718 | 8: 5496138 | 40732 | 8: 5839173 | 40736 | 8: 5906023 |
| 40751 | 8: 6327904 | 40804 | 8: 7593827 | 40861 | 8: 8914108 | 40865 | 8: 9015228 | 40882 | 8: 9392626 |
| 40960 | 8: 11244122 | 40966 | 8: 11330242 | 40968 | 8: 11403633 | 40975 | 8: 11522513 | 40984 | 8: 11668581 |
| 40991 | 8: 11856184 | 40997 | 8: 12018134 | 41009 | 8: 12363854 | 41050 | 8: 13811430 | 41095 | 8: 15117985 |
| 41099 | 8: 15188506 | 41110 | 8: 15477698 | 41112 | 8: 15494200 | 41118 | 8: 15553158 | 41119 | 8: 15574036 |
| 41126 | 8: 15701758 | 41137 | 8: 16168316 | 41138 | 8: 16168775 | 41201 | 8: 17444173 | 41211 | 8: 17638060 |
| 41227 | 8: 18138392 | 41250 | 8: 18931382 | 41279 | 8: 19741400 | 41290 | 8: 19953078 | 41331 | 8: 20945241 |
| 41343 | 8: 21351226 | 41403 | 8: 23473039 | 41410 | 8: 23722262 | 41451 | 8: 24658012 | 41460 | 8: 24730121 |
| 41567 | 8: 25718774 | 41578 | 8: 25952516 | 41581 | 8: 26020143 | 41607 | 8: 26647199 | 41617 | 8: 26902337 |
| 41618 | 8: 26902597 | 41619 | 8: 26902692 | 41620 | 8: 26906751 | 41621 | 8: 26909158 | 41622 | 8: 26909371 |
| 41663 | 8: 28392311 | 41694 | 8: 29260025 | 41703 | 8: 29463858 | 41797 | 8: 31800228 | 41798 | 8: 31843921 |
| 41829 | 8: 32784569 | 41840 | 8: 33342641 | 41853 | 8: 33651679 | 41868 | 8: 34125341 | 41876 | 8: 34286689 |
| 41883 | 8: 34490228 | 41897 | 8: 34815302 | 41907 | 8: 35172469 | 41963 | 8: 36234097 | 41982 | 8: 36725916 |
| 41989 | 8: 36958536 | 42015 | 8: 37563030 | 42052 | 8: 38794675 | 42072 | 8: 39672661 | 42134 | 8: 40784197 |
| 42138 | 8: 40885929 | 42141 | 8: 41023065 | 42185 | 8: 42062463 | 42259 | 8: 44042188 | 42291 | 8: 45099245 |
| 42293 | 8: 45139392 | 42318 | 8: 45729304 | 42324 | 8: 45797263 | 42335 | 8: 46074143 | 42336 | 8: 46102241 |
| 42337 | 8: 46116001 | 42338 | 8: 46116354 | 42339 | 8: 46135520 | 42341 | 8: 46137990 | 42342 | 8: 46144578 |
| 42344 | 8: 46153342 | 42345 | 8: 46156573 | 42375 | 8: 46982936 | 42420 | 8: 48031781 | 42430 | 8: 48479991 |
| 42539 | 8: 51426634 | 42545 | 8: 51576820 | 42575 | 8: 52252692 | 42585 | 8: 52524895 | 42602 | 8: 52976427 |
| 42613 | 8: 53298802 | 42616 | 8: 53382084 | 42702 | 8: 55675444 | 42724 | 8: 56364736 | 42725 | 8: 56366787 |
| 42751 | 8: 56934422 | 42753 | 8: 56941050 | 42754 | 8: 57019107 | 42783 | 8: 57597865 | 42810 | 8: 58618980 |
| 42832 | 8: 59315871 | 42871 | 8: 60125622 | 42934 | 8: 61718348 | 42938 | 8: 61889487 | 42971 | 8: 62566075 |
| 42972 | 8: 62584738 | 42992 | 8: 63183224 | 43052 | 8: 64941395 | 43080 | 8: 65506614 | 43107 | 8: 66205907 |
| 43118 | 8: 66471868 | 43138 | 8: 66967439 | 43144 | 8: 67016183 | 43176 | 8: 67787815 | 43178 | 8: 67793980 |
| 43188 | 8: 67954514 | 43191 | 8: 68086072 | 43206 | 8: 68512363 | 43207 | 8: 68517559 | 43214 | 8: 68740750 |
| 43231 | 8: 69106674 | 43234 | 8: 69118901 | 43266 | 8: 70045486 | 43271 | 8: 70141611 | 43282 | 8: 70418020 |
| 43313 | 8: 71060104 | 43314 | 8: 71090353 | 43325 | 8: 71516540 | 43350 | 8: 72260036 | 43357 | 8: 72432728 |
| 43358 | 8: 72437508 | 43365 | 8: 72557656 | 43388 | 8: 73136540 | 43419 | 8: 73847683 | 43451 | 8: 74692744 |
| 43498 | 8: 76193941 | 43517 | 8: 76534193 | 43528 | 8: 76736188 | 43587 | 8: 78346120 | 43592 | 8: 78443361 |
| 43628 | 8: 79886867 | 43672 | 8: 81022175 | 43675 | 8: 81050611 | 43723 | 8: 82076199 | 43741 | 8: 82402914 |
| 43744 | 8: 82410990 | 43754 | 8: 82684234 | 43775 | 8: 83245271 | 43786 | 8: 83673295 | 43800 | 8: 83846337 |
| 43806 | 8: 83935130 | 43889 | 8: 86085819 | 43893 | 8: 86142301 | 43906 | 8: 86539317 | 43913 | 8: 86746201 |
| 43924 | 8: 86977892 | 43928 | 8: 87127829 | 43929 | 8: 87139627 | 43930 | 8: 87141766 | 43931 | 8: 87142536 |
| 43933 | 8: 87168896 | 43934 | 8: 87175091 | 43935 | 8: 87177155 | 43936 | 8: 87183548 | 43937 | 8: 87200658 |
| 43938 | 8: 87218682 | 43946 | 8: 87439832 | 44026 | 8: 89300462 | 44066 | 8: 90333835 | 44083 | 8: 90850582 |
| 44093 | 8: 90959304 | 44126 | 8: 91835974 | 44162 | 8: 92493864 | 44184 | 8: 93235330 | 44287 | 8: 96565770 |
| 44307 | 8: 97085820 | 44344 | 8: 98025826 | 44346 | 8: 98099120 | 44377 | 8: 98925263 | 44383 | 8: 99073097 |
| 44444 | 8: 100321613 | 44447 | 8: 100390505 | 44521 | 8: 102187006 | 44544 | 8: 102423408 | 44548 | 8: 102462371 |
| 44563 | 8: 102641248 | 44565 | 8: 102643183 | 44575 | 8: 102804781 | 44617 | 8: 103596897 | 44665 | 8: 104455217 |
| 44671 | 8: 104552362 | 44698 | 8: 105230189 | 44708 | 8: 105413829 | 44750 | 8: 106646055 | 44751 | 8: 106656709 |
| 44775 | 8: 107216243 | 44777 | 8: 107231955 | 44791 | 8: 107578002 | 44798 | 8: 107830676 | 44819 | 8: 108486026 |
| 44820 | 8: 108568498 | 44825 | 8: 108815568 | 44854 | 8: 109956440 | 44867 | 8: 110340060 | 44885 | 8: 110872351 |
| 44889 | 8: 110978542 | 44927 | 9: 28156 | 44939 | 9: 575925 | 44940 | 9: 575959 | 44942 | 9: 582062 |
| 44943 | 9: 590791 | 44944 | 9: 593091 | 44945 | 9: 600069 | 44946 | 9: 600393 | 44948 | 9: 618129 |
| 44949 | 9: 632185 | 44950 | 9: 633412 | 44951 | 9: 642243 | 44952 | 9: 659431 | 44953 | 9: 659947 |
| 44954 | 9: 660978 | 44955 | 9: 661495 | 44956 | 9: 665906 | 44958 | 9: 681361 | 44959 | 9: 697171 |
| 44960 | 9: 708137 | 44961 | 9: 709297 | 44964 | 9: 726333 | 44969 | 9: 900985 | 45000 | 9: 1570889 |
| 45010 | 9: 1844977 | 45023 | 9: 2164529 | 45053 | 9: 3003204 | 45069 | 9: 3571118 | 45103 | 9: 4701519 |
| 45114 | 9: 5085846 | 45144 | 9: 5897942 | 45145 | 9: 5911158 | 45148 | 9: 6056118 | 45160 | 9: 6416188 |
| 45193 | 9: 7630506 | 45195 | 9: 7644292 | 45210 | 9: 7983021 | 45214 | 9: 8111645 | 45256 | 9: 9020374 |
| 45257 | 9: 9029730 | 45403 | 9: 11584044 | 45408 | 9: 11752659 | 45448 | 9: 12626683 | 45454 | 9: 12671087 |
| 45455 | 9: 12689678 | 45457 | 9: 12695431 | 45458 | 9: 12695934 | 45459 | 9: 12695941 | 45460 | 9: 12696018 |
| 45461 | 9: 12696113 | 45462 | 9: 12696251 | 45463 | 9: 12696292 | 45464 | 9: 12696440 | 45465 | 9: 12696661 |
| 45466 | 9: 12698815 | 45467 | 9: 12698843 | 45468 | 9: 12700184 | 45472 | 9: 12770979 | 45479 | 9: 13069374 |
| 45480 | 9: 13082121 | 45482 | 9: 13102812 | 45483 | 9: 13103635 | 45484 | 9: 13104067 | 45486 | 9: 13108898 |
| 45487 | 9: 13109246 | 45488 | 9: 13109258 | 45489 | 9: 13110088 | 45494 | 9: 13242607 | 45533 | 9: 14240906 |
| 45547 | 9: 14661048 | 45581 | 9: 15193969 | 45591 | 9: 15445642 | 45614 | 9: 15671238 | 45615 | 9: 15672381 |
| 45616 | 9: 15672435 | 45617 | 9: 15672993 | 45618 | 9: 15673156 | 45619 | 9: 15673271 | 45620 | 9: 15673402 |
| 45621 | 9: 15674725 | 45632 | 9: 15869515 | 45635 | 9: 15935953 | 45639 | 9: 15995486 | 45654 | 9: 16430498 |
| 45663 | 9: 16688073 | 45693 | 9: 17598783 | 45694 | 9: 17599118 | 45783 | 9: 20470091 | 45787 | 9: 20622642 |
| 45794 | 9: 20764203 | 45820 | 9: 21613566 | 45821 | 9: 21636143 | 45822 | 9: 21640871 | 45823 | 9: 21643825 |
| 45825 | 9: 21654717 | 45826 | 9: 21683117 | 45827 | 9: 21683679 | 45829 | 9: 21715481 | 45830 | 9: 21740090 |
| 45831 | 9: 21757906 | 45855 | 9: 22294337 | 45866 | 9: 22593668 | 45869 | 9: 22639958 | 45923 | 9: 23824584 |
| 45927 | 9: 23893279 | 45955 | 9: 24560985 | 45994 | 9: 25737610 | 45995 | 9: 25738079 | 45996 | 9: 25741377 |
| 46014 | 9: 26124495 | 46025 | 9: 26374532 | 46056 | 9: 27294641 | 46078 | 9: 27733478 | 46081 | 9: 27779272 |
| 46083 | 9: 27801667 | 46103 | 9: 28362224 | 46104 | 9: 28363030 | 46113 | 9: 28567312 | 46114 | 9: 28568804 |
| 46115 | 9: 28569972 | 46116 | 9: 28591418 | 46117 | 9: 28593815 | 46122 | 9: 28646308 | 46140 | 9: 29091195 |
| 46143 | 9: 29192292 | 46144 | 9: 29204130 | 46145 | 9: 29256950 | 46147 | 9: 29283806 | 46148 | 9: 29295585 |
| 46151 | 9: 29380299 | 46152 | 9: 29380683 | 46153 | 9: 29381237 | 46154 | 9: 29381255 | 46155 | 9: 29383215 |
| 46156 | 9: 29383228 | 46157 | 9: 29383283 | 46158 | 9: 29384303 | 46159 | 9: 29384799 | 46160 | 9: 29385786 |
| 46161 | 9: 29385818 | 46162 | 9: 29386059 | 46163 | 9: 29386376 | 46164 | 9: 29387098 | 46166 | 9: 29400041 |
| 46167 | 9: 29400182 | 46168 | 9: 29402441 | 46169 | 9: 29402650 | 46170 | 9: 29403623 | 46171 | 9: 29409271 |
| 46172 | 9: 29411199 | 46187 | 9: 29691912 | 46188 | 9: 29692738 | 46199 | 9: 30032893 | 46200 | 9: 30033128 |
| 46218 | 9: 30451686 | 46223 | 9: 30612426 | 46225 | 9: 30625188 | 46246 | 9: 31013461 | 46311 | 9: 32638638 |
| 46346 | 9: 33419705 | 46367 | 9: 33960698 | 46387 | 9: 34556125 | 46410 | 9: 35190440 | 46444 | 9: 36105021 |
| 46494 | 9: 37158301 | 46519 | 9: 37870307 | 46552 | 9: 38368263 | 46582 | 9: 38867506 | 46583 | 9: 38875675 |
| 46584 | 9: 38878148 | 46586 | 9: 38894269 | 46637 | 9: 39995374 | 46695 | 9: 41446116 | 46699 | 9: 41532669 |
| 46739 | 9: 42551675 | 46776 | 9: 43626998 | 46784 | 9: 43912263 | 46785 | 9: 43915403 | 46798 | 9: 44230197 |
| 46806 | 9: 44371483 | 46812 | 9: 44533430 | 46816 | 9: 44595447 | 46859 | 9: 46082473 | 46887 | 9: 46922739 |
| 46902 | 9: 47520187 | 46903 | 9: 47553422 | 46907 | 9: 47590125 | 46975 | 9: 49674165 | 46989 | 9: 50119411 |
| 47011 | 9: 50615961 | 47022 | 9: 50886609 | 47024 | 9: 50944146 | 47064 | 9: 52161063 | 47071 | 9: 52377348 |
| 47106 | 9: 53217250 | 47107 | 9: 53217253 | 47108 | 9: 53217815 | 47109 | 9: 53217988 | 47110 | 9: 53225739 |
| 47111 | 9: 53226122 | 47112 | 9: 53226145 | 47113 | 9: 53227113 | 47114 | 9: 53234782 | 47115 | 9: 53245948 |
| 47116 | 9: 53269708 | 47118 | 9: 53277095 | 47119 | 9: 53298978 | 47163 | 9: 54707994 | 47167 | 9: 54805840 |
| 47180 | 9: 55233499 | 47206 | 9: 56084986 | 47274 | 9: 57805504 | 47290 | 9: 58371472 | 47310 | 9: 58992635 |
| 47323 | 9: 59401110 | 47324 | 9: 59402335 | 47327 | 9: 59422003 | 47329 | 9: 59459837 | 47373 | 9: 60362161 |
| 47469 | 9: 63010039 | 47474 | 9: 63179604 | 47477 | 9: 63266632 | 47524 | 9: 64700448 | 47593 | 9: 65489746 |
| 47601 | 9: 65582850 | 47619 | 9: 66094001 | 47639 | 9: 66606853 | 47652 | 9: 67044823 | 47659 | 9: 67241727 |
| 47664 | 9: 67366039 | 47677 | 9: 67697561 | 47689 | 9: 68036049 | 47693 | 9: 68114580 | 47694 | 9: 68115586 |
| 47713 | 9: 68594580 | 47753 | 9: 69635715 | 47757 | 9: 69752727 | 47788 | 9: 70415891 | 47795 | 9: 70619172 |
| 47807 | 9: 70987356 | 47808 | 9: 70997161 | 47878 | 9: 72597976 | 47889 | 9: 72874182 | 47936 | 9: 74030455 |
| 47949 | 9: 74381423 | 47965 | 9: 74748032 | 48044 | 9: 76966345 | 48070 | 9: 77485895 | 48073 | 9: 77511368 |
| 48075 | 9: 77523509 | 48078 | 9: 77562662 | 48081 | 9: 77694221 | 48083 | 9: 77699388 | 48084 | 9: 77713754 |
| 48085 | 9: 77715782 | 48086 | 9: 77716409 | 48087 | 9: 77716687 | 48088 | 9: 77717203 | 48089 | 9: 77717466 |
| 48090 | 9: 77718722 | 48092 | 9: 77723623 | 48093 | 9: 77728652 | 48094 | 9: 77728956 | 48095 | 9: 77729189 |
| 48096 | 9: 77729397 | 48097 | 9: 77729413 | 48098 | 9: 77730079 | 48099 | 9: 77730486 | 48100 | 9: 77730739 |
| 48101 | 9: 77730950 | 48102 | 9: 77731339 | 48103 | 9: 77731548 | 48104 | 9: 77731587 | 48105 | 9: 77731649 |
| 48106 | 9: 77732824 | 48107 | 9: 77733602 | 48108 | 9: 77733985 | 48109 | 9: 77734457 | 48110 | 9: 77734537 |
| 48111 | 9: 77734632 | 48112 | 9: 77739942 | 48113 | 9: 77740756 | 48114 | 9: 77741688 | 48115 | 9: 77742632 |
| 48116 | 9: 77743840 | 48117 | 9: 77744601 | 48118 | 9: 77744620 | 48119 | 9: 77745039 | 48120 | 9: 77745584 |
| 48121 | 9: 77745608 | 48122 | 9: 77751329 | 48123 | 9: 77751755 | 48124 | 9: 77754808 | 48125 | 9: 77756169 |
| 48126 | 9: 77756446 | 48127 | 9: 77756531 | 48128 | 9: 77762645 | 48129 | 9: 77764362 | 48132 | 9: 77792668 |
| 48133 | 9: 77804673 | 48134 | 9: 77811111 | 48135 | 9: 77811220 | 48136 | 9: 77811579 | 48137 | 9: 77811594 |
| 48138 | 9: 77811612 | 48139 | 9: 77811647 | 48140 | 9: 77811795 | 48141 | 9: 77812291 | 48142 | 9: 77812385 |
| 48143 | 9: 77812527 | 48144 | 9: 77812542 | 48145 | 9: 77812555 | 48146 | 9: 77812585 | 48191 | 9: 79533173 |
| 48196 | 9: 79702375 | 48249 | 9: 80821611 | 48264 | 9: 81093450 | 48265 | 9: 81096096 | 48282 | 9: 81398472 |
| 48298 | 9: 81743991 | 48302 | 9: 81797320 | 48321 | 9: 82185927 | 48326 | 9: 82275767 | 48341 | 9: 82639891 |
| 48346 | 9: 82675266 | 48347 | 9: 82681644 | 48349 | 9: 82685499 | 48350 | 9: 82696652 | 48351 | 9: 82697299 |
| 48352 | 9: 82699229 | 48353 | 9: 82699333 | 48355 | 9: 82706831 | 48356 | 9: 82708646 | 48366 | 9: 82981739 |
| 48375 | 9: 83266367 | 48408 | 9: 84082840 | 48409 | 9: 84084042 | 48410 | 9: 84084459 | 48411 | 9: 84098275 |
| 48412 | 9: 84100341 | 48413 | 9: 84101062 | 48419 | 9: 84280123 | 48430 | 9: 84602199 | 48453 | 9: 85206226 |
| 48527 | 9: 86728832 | 48542 | 9: 87041634 | 48554 | 9: 87334131 | 48645 | 9: 88942711 | 48697 | 9: 89633025 |
| 48704 | 9: 89813410 | 48724 | 9: 90315206 | 48740 | 9: 90776042 | 48765 | 9: 91141962 | 48791 | 9: 91739734 |
| 48805 | 9: 92056243 | 48807 | 9: 92137059 | 48974 | 9: 95252173 | 48981 | 9: 95442777 | 49012 | 9: 96258094 |
| 49084 | 9: 97052004 | 49086 | 9: 97068291 | 49093 | 9: 97195333 | 49115 | 9: 97614153 | 49118 | 9: 97651483 |
| 49158 | 9: 98444344 | 49165 | 9: 98565031 | 49183 | 9: 99022247 | 49194 | 9: 99214577 | 49227 | 9: 99757934 |
| 49228 | 9: 99766501 | 49286 | 9: 100838600 | 49287 | 9: 100865957 | 49301 | 9: 101130910 | 49308 | 9: 101233972 |
| 49316 | 9: 101405391 | 49323 | 9: 101487283 | 49399 | 9: 102585986 | 49428 | 9: 102905463 | 49443 | 9: 103267046 |
| 49470 | 9: 103818635 | 49557 | 10: 1514567 | 49560 | 10: 1608093 | 49571 | 10: 1809005 | 49573 | 10: 1812942 |
| 49575 | 10: 1815628 | 49596 | 10: 2181089 | 49600 | 10: 2318770 | 49603 | 10: 2355906 | 49633 | 10: 2956009 |
| 49650 | 10: 3286732 | 49685 | 10: 3957621 | 49719 | 10: 4637364 | 49756 | 10: 5504897 | 49757 | 10: 5506239 |
| 49758 | 10: 5506479 | 49759 | 10: 5506633 | 49760 | 10: 5507158 | 49761 | 10: 5507326 | 49762 | 10: 5507349 |
| 49763 | 10: 5507472 | 49764 | 10: 5507500 | 49765 | 10: 5507611 | 49766 | 10: 5507931 | 49767 | 10: 5507997 |
| 49768 | 10: 5508125 | 49769 | 10: 5508230 | 49770 | 10: 5509205 | 49771 | 10: 5509288 | 49772 | 10: 5511487 |
| 49773 | 10: 5511551 | 49774 | 10: 5511726 | 49817 | 10: 6459980 | 49835 | 10: 6823353 | 49837 | 10: 6940126 |
| 49899 | 10: 8330854 | 49901 | 10: 8385791 | 49917 | 10: 8566513 | 49965 | 10: 9790239 | 49972 | 10: 9846509 |
| 49987 | 10: 10261088 | 50046 | 10: 11321931 | 50047 | 10: 11322196 | 50076 | 10: 12039647 | 50090 | 10: 12326160 |
| 50091 | 10: 12337486 | 50093 | 10: 12394358 | 50099 | 10: 12500777 | 50121 | 10: 13059560 | 50124 | 10: 13141850 |
| 50132 | 10: 13207866 | 50133 | 10: 13222364 | 50134 | 10: 13224497 | 50135 | 10: 13225359 | 50136 | 10: 13226008 |
| 50139 | 10: 13238081 | 50140 | 10: 13248003 | 50142 | 10: 13255208 | 50143 | 10: 13258421 | 50144 | 10: 13282620 |
| 50145 | 10: 13286682 | 50149 | 10: 13347534 | 50150 | 10: 13347535 | 50179 | 10: 13823541 | 50183 | 10: 13888930 |
| 50189 | 10: 13964255 | 50193 | 10: 14079328 | 50212 | 10: 14629981 | 50215 | 10: 14653991 | 50257 | 10: 15551922 |
| 50260 | 10: 15625431 | 50298 | 10: 16237750 | 50328 | 10: 16751980 | 50350 | 10: 17119224 | 50352 | 10: 17147669 |
| 50367 | 10: 17432966 | 50379 | 10: 17732565 | 50396 | 10: 18087252 | 50397 | 10: 18089168 | 50398 | 10: 18089791 |
| 50399 | 10: 18089811 | 50400 | 10: 18092432 | 50401 | 10: 18095170 | 50402 | 10: 18096798 | 50403 | 10: 18100952 |
| 50404 | 10: 18105123 | 50411 | 10: 18246343 | 50458 | 10: 19161271 | 50477 | 10: 19610258 | 50508 | 10: 20302691 |
| 50514 | 10: 20365815 | 50579 | 10: 21377001 | 50615 | 10: 22113385 | 50620 | 10: 22271591 | 50709 | 10: 26380149 |
| 50714 | 10: 26474079 | 50715 | 10: 26474882 | 50741 | 10: 26820943 | 50777 | 10: 27459845 | 50848 | 10: 28936817 |
| 50850 | 10: 28939598 | 50861 | 10: 29148690 | 50903 | 10: 29990871 | 50973 | 10: 32272335 | 50977 | 10: 32347626 |
| 50980 | 10: 32449180 | 50984 | 10: 32556195 | 51013 | 10: 33127535 | 51014 | 10: 33187873 | 51029 | 10: 33566653 |
| 51086 | 10: 34900072 | 51089 | 10: 34926072 | 51095 | 10: 35028845 | 51152 | 10: 36423193 | 51178 | 10: 37020667 |
| 51199 | 10: 37655975 | 51203 | 10: 37686916 | 51241 | 10: 38641559 | 51258 | 10: 39024264 | 51342 | 10: 41413651 |
| 51351 | 10: 41730162 | 51352 | 10: 41731424 | 51359 | 10: 41898236 | 51375 | 10: 42389264 | 51378 | 10: 42430118 |
| 51380 | 10: 42448787 | 51382 | 10: 42452483 | 51383 | 10: 42455131 | 51389 | 10: 42580900 | 51404 | 10: 43179077 |
| 51419 | 10: 43825008 | 51479 | 10: 45276327 | 51526 | 10: 46079546 | 51575 | 10: 47154407 | 51602 | 10: 47651824 |
| 51624 | 10: 48309146 | 51656 | 10: 48956010 | 51683 | 10: 49439054 | 51714 | 10: 50089231 | 51803 | 10: 52130146 |
| 51819 | 10: 52557986 | 51853 | 10: 53459983 | 51878 | 10: 54196187 | 51893 | 10: 54537058 | 51894 | 10: 54542851 |
| 51931 | 10: 55708254 | 51954 | 10: 56299639 | 51978 | 10: 56721716 | 51981 | 10: 56843229 | 51982 | 10: 56922663 |
| 51988 | 10: 57043405 | 51991 | 10: 57115514 | 52018 | 10: 57976067 | 52081 | 10: 59624803 | 52102 | 10: 60442020 |
| 52108 | 10: 60636369 | 52159 | 10: 62050176 | 52174 | 10: 62290589 | 52249 | 10: 64233969 | 52267 | 10: 64525088 |
| 52268 | 10: 64536219 | 52269 | 10: 64543768 | 52270 | 10: 64548032 | 52271 | 10: 64548906 | 52286 | 10: 64979230 |
| 52295 | 10: 65266598 | 52339 | 10: 66097225 | 52340 | 10: 66120437 | 52361 | 10: 66674133 | 52362 | 10: 66674580 |
| 52363 | 10: 66675048 | 52364 | 10: 66675130 | 52366 | 10: 66770244 | 52422 | 10: 68107692 | 52424 | 10: 68140093 |
| 52444 | 10: 68518650 | 52536 | 10: 71021997 | 52557 | 10: 71661492 | 52574 | 10: 72103748 | 52600 | 10: 73153480 |
| 52604 | 10: 73217430 | 52615 | 10: 73492848 | 52620 | 10: 73620458 | 52681 | 10: 75374747 | 52696 | 10: 75774470 |
| 52699 | 10: 75886187 | 52707 | 10: 76141481 | 52708 | 10: 76174432 | 52734 | 10: 76609622 | 52735 | 10: 76613357 |
| 52737 | 10: 76718459 | 52746 | 10: 76886184 | 52808 | 10: 78222151 | 52816 | 10: 78333529 | 52831 | 10: 78834237 |
| 52838 | 10: 78993174 | 52847 | 10: 79256209 | 52855 | 10: 79448706 | 52875 | 10: 80033473 | 52893 | 10: 80437796 |
| 52905 | 10: 80755095 | 52952 | 10: 81403359 | 53025 | 10: 82946024 | 53076 | 10: 84003158 | 53119 | 10: 84929419 |
| 53136 | 10: 85211692 | 53141 | 10: 85308316 | 53146 | 10: 85365819 | 53151 | 10: 85506776 | 53153 | 10: 85532563 |
| 53180 | 10: 86322591 | 53190 | 10: 86480002 | 53198 | 10: 86564397 | 53234 | 10: 87380593 | 53240 | 10: 87570845 |
| 53303 | 10: 88984928 | 53307 | 10: 89076810 | 53328 | 10: 89477438 | 53338 | 10: 89716618 | 53376 | 10: 90408131 |
| 53386 | 10: 90606603 | 53387 | 10: 90610286 | 53410 | 10: 90869492 | 53412 | 10: 90878413 | 53427 | 10: 91270590 |
| 53500 | 10: 92937814 | 53506 | 10: 93026084 | 53510 | 10: 93101284 | 53522 | 10: 93436468 | 53527 | 10: 93605955 |
| 53581 | 10: 94985373 | 53592 | 10: 95290678 | 53606 | 10: 95624360 | 53629 | 10: 96068605 | 53633 | 10: 96193408 |
| 53649 | 10: 96591095 | 53654 | 10: 96786131 | 53655 | 10: 96788386 | 53694 | 10: 97467089 | 53707 | 10: 97772610 |
| 53708 | 10: 97775801 | 53709 | 10: 97778298 | 53734 | 10: 98439866 | 53759 | 10: 98730218 | 53782 | 10: 99444853 |
| 53785 | 10: 99471490 | 53794 | 10: 99596036 | 53811 | 10: 99982401 | 53873 | 10: 10113952 | 53898 | 10: 10165041 |
| 53899 | 10: 10166055 | 53900 | 10: 10167602 | 53902 | 10: 10170386 | 53916 | 10: 10185170 | 53926 | 10: 10206511 |
| 53928 | 10: 10208682 | 53933 | 10: 10213240 | 53939 | 10: 10223950 | 53977 | 10: 10315226 | 54020 | 11: 779732 |
| 54030 | 11: 923580 | 54035 | 11: 1009994 | 54047 | 11: 1211969 | 54118 | 11: 2084360 | 54127 | 11: 2256142 |
| 54133 | 11: 2359945 | 54181 | 11: 2799677 | 54196 | 11: 3053638 | 54206 | 11: 3153964 | 54238 | 11: 3763768 |
| 54328 | 11: 5718123 | 54345 | 11: 6071587 | 54380 | 11: 7143509 | 54398 | 11: 7527861 | 54404 | 11: 7599092 |
| 54441 | 11: 8559109 | 54503 | 11: 9819964 | 54512 | 11: 10000902 | 54588 | 11: 11731827 | 54594 | 11: 11844298 |
| 54617 | 11: 12454930 | 54653 | 11: 13209044 | 54654 | 11: 13222009 | 54689 | 11: 13914152 | 54696 | 11: 14200577 |
| 54727 | 11: 15182386 | 54732 | 11: 15285660 | 54749 | 11: 15700826 | 54770 | 11: 16056267 | 54804 | 11: 16934584 |
| 54814 | 11: 17117515 | 54867 | 11: 18770128 | 54928 | 11: 20656584 | 54943 | 11: 21060821 | 54992 | 11: 22018210 |
| 55000 | 11: 22303794 | 55012 | 11: 22569464 | 55020 | 11: 22724476 | 55032 | 11: 23083144 | 55068 | 11: 23895849 |
| 55087 | 11: 24338666 | 55097 | 11: 24469097 | 55129 | 11: 25273777 | 55140 | 11: 25560609 | 55162 | 11: 26154618 |
| 55165 | 11: 26170393 | 55252 | 11: 28239112 | 55304 | 11: 29324448 | 55317 | 11: 29530060 | 55324 | 11: 29724736 |
| 55329 | 11: 29759229 | 55355 | 11: 30470011 | 55385 | 11: 31068333 | 55434 | 11: 32501555 | 55466 | 11: 33302958 |
| 55503 | 11: 34519584 | 55542 | 11: 35824586 | 55552 | 11: 36039319 | 55557 | 11: 36151963 | 55562 | 11: 36293996 |
| 55577 | 11: 36723444 | 55599 | 11: 37215399 | 55600 | 11: 37240873 | 55626 | 11: 37656828 | 55634 | 11: 37841103 |
| 55637 | 11: 37898906 | 55672 | 11: 39016826 | 55701 | 11: 40047215 | 55706 | 11: 40222392 | 55716 | 11: 40663028 |
| 55751 | 11: 41311710 | 55752 | 11: 41326914 | 55755 | 11: 41328623 | 55758 | 11: 41354523 | 55760 | 11: 41356073 |
| 55766 | 11: 41457760 | 55774 | 11: 41843798 | 55796 | 11: 42373804 | 55797 | 11: 42374333 | 55798 | 11: 42374619 |
| 55799 | 11: 42374860 | 55800 | 11: 42375434 | 55802 | 11: 42378608 | 55803 | 11: 42395653 | 55804 | 11: 42395937 |
| 55808 | 11: 42450574 | 55809 | 11: 42458071 | 55826 | 11: 42952869 | 55828 | 11: 42977381 | 55834 | 11: 43155397 |
| 55849 | 11: 43447636 | 55855 | 11: 43656266 | 55867 | 11: 44164688 | 55871 | 11: 44197049 | 55904 | 11: 45053290 |
| 55950 | 11: 46193646 | 55973 | 11: 46703688 | 55976 | 11: 46763745 | 55977 | 11: 46763771 | 55978 | 11: 46763772 |
| 55979 | 11: 46763803 | 55980 | 11: 46763967 | 55981 | 11: 46764372 | 55982 | 11: 46765309 | 55983 | 11: 46769693 |
| 56013 | 11: 47198478 | 56053 | 11: 48210329 | 56122 | 11: 48963797 | 56127 | 11: 49061005 | 56128 | 11: 49080138 |
| 56135 | 11: 49179291 | 56138 | 11: 49285957 | 56143 | 11: 49404465 | 56147 | 11: 49422373 | 56152 | 11: 49575365 |
| 56153 | 11: 49582523 | 56198 | 11: 51041604 | 56201 | 11: 51160828 | 56205 | 11: 51255454 | 56206 | 11: 51261576 |
| 56252 | 11: 53012268 | 56253 | 11: 53022205 | 56257 | 11: 53214759 | 56272 | 11: 53823211 | 56293 | 11: 54411745 |
| 56301 | 11: 54607804 | 56302 | 11: 54611060 | 56303 | 11: 54615185 | 56304 | 11: 54619006 | 56305 | 11: 54619299 |
| 56306 | 11: 54621378 | 56307 | 11: 54624272 | 56308 | 11: 54625736 | 56309 | 11: 54637534 | 56310 | 11: 54640117 |
| 56311 | 11: 54640249 | 56312 | 11: 54644177 | 56313 | 11: 54658429 | 56314 | 11: 54667025 | 56315 | 11: 54672210 |
| 56317 | 11: 54687119 | 56318 | 11: 54696136 | 56327 | 11: 54925623 | 56341 | 11: 55201147 | 56345 | 11: 55322961 |
| 56384 | 11: 56468221 | 56394 | 11: 56809772 | 56395 | 11: 56811610 | 56396 | 11: 56812388 | 56408 | 11: 57181716 |
| 56434 | 11: 58004110 | 56441 | 11: 58172817 | 56451 | 11: 58552292 | 56452 | 11: 58557598 | 56478 | 11: 59155112 |
| 56498 | 11: 59762890 | 56519 | 11: 60453153 | 56528 | 11: 60471884 | 56539 | 11: 60491356 | 56609 | 11: 62266387 |
| 56611 | 11: 62305455 | 56612 | 11: 62308757 | 56614 | 11: 62379499 | 56617 | 11: 62426969 | 56623 | 11: 62609754 |
| 56681 | 11: 63826838 | 56692 | 11: 63976694 | 56707 | 11: 64337696 | 56711 | 11: 64464647 | 56737 | 11: 65172790 |
| 56789 | 11: 66422364 | 56887 | 11: 68143046 | 56912 | 11: 68896819 | 56918 | 11: 69065684 | 56919 | 11: 69065947 |
| 56920 | 11: 69067967 | 56927 | 11: 69141312 | 56934 | 11: 69198908 | 56936 | 11: 69213888 | 56965 | 11: 69725037 |
| 56975 | 11: 69945179 | 56979 | 11: 70105382 | 56989 | 11: 70270717 | 56991 | 11: 70291428 | 57061 | 11: 71167695 |
| 57063 | 11: 71169475 | 57064 | 11: 71171201 | 57065 | 11: 71174044 | 57071 | 11: 71187848 | 57083 | 11: 71410081 |
| 57092 | 11: 71596359 | 57093 | 11: 71603815 | 57097 | 11: 71735550 | 57118 | 11: 72456335 | 57127 | 11: 72799464 |
| 57171 | 11: 73648343 | 57267 | 11: 75692626 | 57288 | 11: 76333610 | 57314 | 11: 77013992 | 57318 | 11: 77067925 |
| 57319 | 11: 77077199 | 57320 | 11: 77085172 | 57330 | 11: 77232878 | 57340 | 11: 77445226 | 57341 | 11: 77485470 |
| 57396 | 11: 78194981 | 57405 | 11: 78326150 | 57555 | 11: 81830178 | 57571 | 11: 82150919 | 57595 | 11: 82670314 |
| 57617 | 11: 82895182 | 57620 | 11: 82943647 | 57646 | 11: 83482447 | 57648 | 11: 83552676 | 57708 | 11: 85258209 |
| 57718 | 11: 85436750 | 57725 | 11: 85653522 | 57730 | 11: 85743559 | 57754 | 11: 86206294 | 57755 | 11: 86214871 |
| 57814 | 11: 87707465 | 57847 | 11: 87999059 | 57865 | 11: 88032183 | 57884 | 11: 88448012 | 57927 | 11: 89546657 |
| 57946 | 11: 89996070 | 57970 | 11: 90503652 | 58023 | 11: 91710069 | 58027 | 11: 91791559 | 58028 | 11: 91792786 |
| 58029 | 11: 91796326 | 58030 | 11: 91798254 | 58031 | 11: 91807646 | 58032 | 11: 91809950 | 58033 | 11: 91809976 |
| 58042 | 11: 92019924 | 58082 | 11: 92823026 | 58091 | 11: 93153121 | 58092 | 11: 93165769 | 58093 | 11: 93179602 |
| 58094 | 11: 93185994 | 58100 | 11: 93232642 | 58105 | 11: 93261434 | 58106 | 11: 93262306 | 58107 | 11: 93262884 |
| 58108 | 11: 93263825 | 58109 | 11: 93279097 | 58111 | 11: 93395136 | 58124 | 11: 93858711 | 58143 | 11: 94337656 |
| 58163 | 11: 94860855 | 58168 | 11: 95063641 | 58175 | 11: 95296518 | 58268 | 11: 97728887 | 58299 | 11: 98222359 |
| 58303 | 11: 98359510 | 58373 | 11: 99755436 | 58492 | 11: 10201461 | 58517 | 11: 10257709 | 58524 | 11: 10265965 |
| 58583 | 11: 10338389 | 58596 | 11: 10369741 | 58630 | 11: 10447397 | 58644 | 11: 10468322 | 58654 | 11: 10482995 |
| 58656 | 11: 10487555 | 58658 | 11: 10492751 | 58661 | 11: 10497336 | 58663 | 11: 10497439 | 58681 | 11: 10548246 |
| 58729 | 11: 10679315 | 58731 | 11: 10694859 | 58752 | 12: 625880 | 58780 | 12: 1498811 | 58821 | 12: 2369495 |
| 58870 | 12: 3405573 | 58875 | 12: 3491002 | 58894 | 12: 4026056 | 58925 | 12: 4736478 | 58934 | 12: 5016508 |
| 58960 | 12: 5810001 | 59000 | 12: 7239138 | 59032 | 12: 8559085 | 59098 | 12: 10166200 | 59115 | 12: 10466559 |
| 59148 | 12: 11055011 | 59172 | 12: 11660529 | 59177 | 12: 11743244 | 59237 | 12: 13369400 | 59275 | 12: 14073643 |
| 59284 | 12: 14200873 | 59334 | 12: 15275995 | 59404 | 12: 16588996 | 59405 | 12: 16617595 | 59445 | 12: 17717003 |
| 59511 | 12: 19127423 | 59512 | 12: 19133184 | 59553 | 12: 20149260 | 59563 | 12: 20389759 | 59565 | 12: 20491988 |
| 59566 | 12: 20500610 | 59608 | 12: 21574455 | 59700 | 12: 23970417 | 59718 | 12: 24380235 | 59730 | 12: 24612900 |
| 59753 | 12: 25107687 | 59760 | 12: 25333357 | 59778 | 12: 25727678 | 59779 | 12: 25742060 | 59782 | 12: 25755570 |
| 59787 | 12: 25862626 | 59816 | 12: 26904714 | 59834 | 12: 27216744 | 59891 | 12: 28914720 | 59952 | 12: 30585785 |
| 59960 | 12: 30737586 | 59964 | 12: 30851151 | 59965 | 12: 30851590 | 59978 | 12: 31055012 | 60064 | 12: 33121234 |
| 60068 | 12: 33212754 | 60115 | 12: 34706439 | 60158 | 12: 35595816 | 60205 | 12: 37118156 | 60252 | 12: 39318432 |
| 60253 | 12: 39337804 | 60254 | 12: 39339971 | 60255 | 12: 39375252 | 60295 | 12: 40436881 | 60299 | 12: 40699170 |
| 60310 | 12: 41000477 | 60345 | 12: 42446263 | 60346 | 12: 42529266 | 60384 | 12: 44349627 | 60414 | 12: 45309570 |
| 60464 | 12: 46902819 | 60503 | 12: 47894827 | 60512 | 12: 48234155 | 60524 | 12: 48552141 | 60528 | 12: 48621944 |
| 60557 | 12: 49347591 | 60588 | 12: 50012484 | 60654 | 12: 51413041 | 60677 | 12: 52057233 | 60712 | 12: 52833730 |
| 60713 | 12: 52841272 | 60714 | 12: 52843772 | 60715 | 12: 52859350 | 60717 | 12: 52867407 | 60718 | 12: 52868520 |
| 60720 | 12: 52883259 | 60721 | 12: 52888503 | 60736 | 12: 53324225 | 60737 | 12: 53326935 | 60748 | 12: 53513237 |
| 60776 | 12: 54070308 | 60778 | 12: 54112016 | 60800 | 12: 54537212 | 60823 | 12: 55077299 | 60834 | 12: 55543664 |
| 60864 | 12: 56465958 | 60881 | 12: 57046134 | 60949 | 12: 59148039 | 60950 | 12: 59163748 | 60958 | 12: 59442298 |
| 61087 | 12: 63768535 | 61092 | 12: 63878306 | 61108 | 12: 64352510 | 61118 | 12: 64738377 | 61196 | 12: 66786596 |
| 61240 | 12: 67928955 | 61247 | 12: 68098257 | 61248 | 12: 68114447 | 61259 | 12: 68270144 | 61275 | 12: 68608809 |
| 61278 | 12: 68710557 | 61290 | 12: 68863135 | 61301 | 12: 69019005 | 61309 | 12: 69286353 | 61311 | 12: 69340936 |
| 61318 | 12: 69467691 | 61328 | 12: 69553759 | 61332 | 12: 69582989 | 61366 | 12: 71235763 | 61382 | 12: 71600338 |
| 61412 | 12: 72958303 | 61413 | 12: 72961991 | 61446 | 12: 73518731 | 61482 | 12: 74197569 | 61497 | 12: 74496338 |
| 61522 | 12: 75101487 | 61529 | 12: 75242221 | 61532 | 12: 75277582 | 61537 | 12: 75375680 | 61590 | 12: 76682892 |
| 61618 | 12: 77393088 | 61638 | 12: 77847200 | 61645 | 12: 77993107 | 61673 | 12: 78662420 | 61704 | 12: 79284630 |
| 61718 | 12: 79542780 | 61728 | 12: 79738869 | 61749 | 12: 80221064 | 61750 | 12: 80223568 | 61751 | 12: 80224696 |
| 51755 | 12: 80311844 | 61760 | 12: 80405755 | 61762 | 12: 80523810 | 61818 | 12: 81778398 | 61836 | 12: 82156928 |
| 61850 | 12: 82530033 | 61865 | 12: 82796759 | 61888 | 12: 83319494 | 61922 | 12: 84042281 | 61956 | 12: 84892891 |
| 61968 | 12: 85033607 | 61980 | 12: 85234076 | 61998 | 12: 85446719 | 62009 | 12: 85641586 | 62011 | 12: 85752928 |
| 62060 | 12: 86779904 | 62103 | 13: 648927 | 62135 | 13: 1384950 | 62142 | 13: 1583266 | 62183 | 13: 2254372 |
| 62213 | 13: 2708064 | 62260 | 13: 3861084 | 62336 | 13: 5664259 | 62337 | 13: 5666787 | 62361 | 13: 6275220 |
| 62366 | 13: 6361450 | 62408 | 13: 7483031 | 62413 | 13: 7556779 | 62434 | 13: 8333741 | 62438 | 13: 8415584 |
| 62443 | 13: 8529126 | 62444 | 13: 8533157 | 62445 | 13: 8537080 | 62446 | 13: 8542772 | 62450 | 13: 8682255 |
| 62451 | 13: 8682878 | 62452 | 13: 8683708 | 62453 | 13: 8685414 | 62454 | 13: 8686278 | 62524 | 13: 10400290 |
| 62526 | 13: 10426820 | 62537 | 13: 10575199 | 62553 | 13: 11047542 | 62573 | 13: 12140250 | 62613 | 13: 13240348 |
| 62654 | 13: 14380630 | 62665 | 13: 14649538 | 62669 | 13: 14768979 | 62675 | 13: 14878218 | 62690 | 13: 15298554 |
| 62693 | 13: 15509230 | 62707 | 13: 15907128 | 62845 | 13: 18649928 | 62849 | 13: 18704268 | 62880 | 13: 18741567 |
| 62886 | 13: 18795212 | 62893 | 13: 18926634 | 62904 | 13: 19113938 | 62927 | 13: 19611692 | 62953 | 13: 19991782 |
| 62955 | 13: 20085636 | 62962 | 13: 20299025 | 62985 | 13: 20633696 | 62991 | 13: 20728403 | 62999 | 13: 21031730 |
| 63014 | 13: 21160488 | 63016 | 13: 21199075 | 63039 | 13: 21675803 | 63072 | 13: 22491633 | 63080 | 13: 22645333 |
| 63142 | 13: 24652712 | 63154 | 13: 24964331 | 63170 | 13: 25313389 | 63233 | 13: 26864651 | 63296 | 13: 28352469 |
| 63310 | 13: 28677951 | 63311 | 13: 28716131 | 63313 | 13: 28719397 | 63314 | 13: 28721486 | 63315 | 13: 28726672 |
| 63328 | 13: 29164280 | 63331 | 13: 29203677 | 63409 | 13: 30734642 | 63413 | 13: 30835419 | 63417 | 13: 30928296 |
| 63433 | 13: 31170980 | 63479 | 13: 32132576 | 63480 | 13: 32133958 | 63481 | 13: 32143886 | 63482 | 13: 32152278 |
| 63494 | 13: 32274709 | 63506 | 13: 32489566 | 63521 | 13: 32651702 | 63522 | 13: 32653621 | 63523 | 13: 32654103 |
| 63528 | 13: 32846791 | 63529 | 13: 32846825 | 63530 | 13: 32847172 | 63531 | 13: 32847400 | 63542 | 13: 33084958 |
| 63543 | 13: 33086348 | 63572 | 13: 33880293 | 63599 | 13: 34800113 | 63619 | 13: 35179138 | 63622 | 13: 35197458 |
| 63766 | 13: 38636130 | 63767 | 13: 38662619 | 63768 | 13: 38676202 | 63769 | 13: 38680784 | 63775 | 13: 38810147 |
| 63781 | 13: 38839160 | 63782 | 13: 38893419 | 63786 | 13: 38949069 | 63796 | 13: 39090329 | 63800 | 13: 39121053 |
| 63832 | 13: 39696126 | 63852 | 13: 40100334 | 63860 | 13: 40220102 | 63870 | 13: 40453192 | 63872 | 13: 40473511 |
| 63873 | 13: 40486594 | 63874 | 13: 40486661 | 63940 | 13: 42071486 | 63972 | 13: 42587452 | 63982 | 13: 42606861 |
| 64003 | 13: 42825481 | 64024 | 13: 43516130 | 64032 | 13: 43689434 | 64033 | 13: 43726105 | 64039 | 13: 43774352 |
| 64059 | 13: 44057830 | 64060 | 13: 44115215 | 64105 | 13: 44933952 | 64122 | 13: 44991547 | 64157 | 13: 45403608 |
| 64176 | 13: 45936548 | 64210 | 13: 46658312 | 64216 | 13: 46758989 | 64245 | 13: 47366925 | 64250 | 13: 47472066 |
| 64254 | 13: 47495500 | 64264 | 13: 47827146 | 64284 | 13: 48238821 | 64285 | 13: 48240137 | 64286 | 13: 48240290 |
| 64287 | 13: 48240322 | 64288 | 13: 48240351 | 64289 | 13: 48241221 | 64290 | 13: 48241373 | 64291 | 13: 48241390 |
| 64292 | 13: 48243603 | 64293 | 13: 48245013 | 64294 | 13: 48245053 | 64296 | 13: 48269729 | 64297 | 13: 48274446 |
| 64318 | 13: 49199089 | 64319 | 13: 49199105 | 64320 | 13: 49200231 | 64419 | 13: 52063310 | 64429 | 13: 52466434 |
| 64496 | 13: 53368735 | 64499 | 13: 53503066 | 64516 | 13: 54246719 | 64522 | 13: 54512037 | 64533 | 13: 54732415 |
| 64593 | 13: 56026065 | 64649 | 13: 57529999 | 64650 | 13: 57541123 | 64669 | 13: 58019158 | 64698 | 13: 58633062 |
| 64708 | 13: 58899200 | 64721 | 13: 59016374 | 64732 | 13: 59248087 | 64735 | 13: 59289762 | 64766 | 13: 60057972 |
| 64776 | 13: 60212933 | 64798 | 13: 60664451 | 64807 | 13: 60980294 | 64822 | 13: 61522788 | 64846 | 13: 62118304 |
| 64852 | 13: 62277677 | 64888 | 13: 62739693 | 64889 | 13: 62752247 | 64893 | 13: 62800301 | 64899 | 13: 62843052 |
| 64946 | 13: 63972761 | 64967 | 13: 64523897 | 64988 | 13: 65061778 | 65005 | 13: 65480528 | 65011 | 13: 65604335 |
| 65014 | 13: 65656406 | 65021 | 13: 65714427 | 65023 | 13: 65715682 | 65033 | 13: 65806803 | 65052 | 13: 66249739 |
| 65070 | 13: 66636049 | 65076 | 13: 66754479 | 65077 | 13: 66811790 | 65082 | 13: 66930495 | 65099 | 13: 67376538 |
| 65112 | 13: 67740112 | 65114 | 13: 67768319 | 65118 | 13: 67910634 | 65141 | 13: 68545291 | 65177 | 13: 69533712 |
| 65193 | 13: 69946188 | 65219 | 13: 70559101 | 65254 | 13: 71281225 | 65255 | 13: 71297993 | 65264 | 13: 71463110 |
| 65285 | 13: 71937940 | 65291 | 13: 72156689 | 65345 | 13: 73285435 | 65394 | 13: 74233601 | 65400 | 13: 74368669 |
| 65402 | 13: 74389287 | 65435 | 13: 74875799 | 65458 | 13: 75539335 | 65459 | 13: 75546891 | 65460 | 13: 75550544 |
| 65475 | 13: 75773530 | 65490 | 13: 76048819 | 65495 | 13: 76206814 | 65497 | 13: 76256932 | 65513 | 13: 76540633 |
| 65533 | 13: 76870141 | 65610 | 13: 78594569 | 65632 | 13: 78943793 | 65642 | 13: 79095868 | 65658 | 13: 79307341 |
| 65667 | 13: 79411595 | 65668 | 13: 79427565 | 65687 | 13: 79896093 | 65693 | 13: 80009218 | 65699 | 13: 80110062 |
| 65700 | 13: 80110693 | 65701 | 13: 80112377 | 65702 | 13: 80112537 | 65703 | 13: 80112873 | 65704 | 13: 80113035 |
| 65705 | 13: 80113195 | 65706 | 13: 80119481 | 65707 | 13: 80120615 | 65708 | 13: 80121232 | 65709 | 13: 80121378 |
| 65710 | 13: 80121492 | 65711 | 13: 80121527 | 65712 | 13: 80122117 | 65713 | 13: 80123053 | 65714 | 13: 80123496 |
| 65715 | 13: 80123540 | 65716 | 13: 80128525 | 65717 | 13: 80128577 | 65718 | 13: 80128592 | 65719 | 13: 80128724 |
| 65720 | 13: 80133414 | 65726 | 13: 80284611 | 65730 | 13: 80353010 | 65731 | 13: 80354577 | 65741 | 13: 80564883 |
| 65792 | 13: 81650045 | 65813 | 13: 82057542 | 65814 | 13: 82060563 | 65847 | 13: 82692047 | 65937 | 14: 1748354 |
| 65939 | 14: 1780705 | 65960 | 14: 2169492 | 65962 | 14: 2196270 | 65979 | 14: 2821082 | 65999 | 14: 3180090 |
| 66000 | 14: 3180092 | 66001 | 14: 3184037 | 66003 | 14: 3188747 | 66031 | 14: 3341005 | 66057 | 14: 3777934 |
| 66059 | 14: 3807311 | 66065 | 14: 3937653 | 66096 | 14: 4438274 | 66107 | 14: 4826114 | 66109 | 14: 4845905 |
| 66112 | 14: 4900819 | 66114 | 14: 4924180 | 66123 | 14: 5079999 | 66141 | 14: 5424566 | 66143 | 14: 5442736 |
| 66144 | 14: 5442915 | 66155 | 14: 5642922 | 66161 | 14: 5745989 | 66162 | 14: 5746008 | 66163 | 14: 5746079 |
| 66164 | 14: 5746088 | 66167 | 14: 5767279 | 66168 | 14: 5767316 | 66182 | 14: 6051710 | 66222 | 14: 6580835 |
| 66251 | 14: 7127015 | 66255 | 14: 7190153 | 66260 | 14: 7315224 | 66267 | 14: 7446316 | 66296 | 14: 7973769 |
| 66298 | 14: 7981735 | 66299 | 14: 7984017 | 66301 | 14: 7993501 | 66303 | 14: 8000324 | 66306 | 14: 8006167 |
| 66307 | 14: 8008471 | 66309 | 14: 8029440 | 66324 | 14: 8315882 | 66339 | 14: 8555196 | 66342 | 14: 8578594 |
| 66401 | 14: 9370788 | 66424 | 14: 9883880 | 66445 | 14: 10330270 | 66487 | 14: 11125973 | 66519 | 14: 11675980 |
| 66520 | 14: 11687647 | 66521 | 14: 11700111 | 66536 | 14: 12017220 | 66572 | 14: 12683380 | 66573 | 14: 12683869 |
| 66575 | 14: 12685152 | 66576 | 14: 12685153 | 66580 | 14: 12718898 | 66581 | 14: 12726976 | 66582 | 14: 12726981 |
| 66642 | 14: 14433818 | 66674 | 14: 15281409 | 66683 | 14: 15476637 | 66707 | 14: 15871072 | 66740 | 14: 16464011 |
| 66800 | 14: 17774684 | 66848 | 14: 18880176 | 66911 | 14: 20615962 | 66917 | 14: 20673221 | 66920 | 14: 20723405 |
| 66921 | 14: 20728338 | 66939 | 14: 21119658 | 66940 | 14: 21124340 | 66973 | 14: 21853648 | 67075 | 14: 22718406 |
| 67158 | 14: 23199725 | 67159 | 14: 23200416 | 67160 | 14: 23200440 | 67161 | 14: 23202111 | 67162 | 14: 23202373 |
| 67163 | 14: 23202574 | 67164 | 14: 23202642 | 67165 | 14: 23202665 | 67166 | 14: 23205217 | 67167 | 14: 23206165 |
| 67168 | 14: 23209278 | 67169 | 14: 23210217 | 67170 | 14: 23211671 | 67171 | 14: 23212276 | 67172 | 14: 23212424 |
| 67174 | 14: 23216603 | 67176 | 14: 23216967 | 67177 | 14: 23217298 | 67178 | 14: 23217676 | 67179 | 14: 23217682 |
| 67182 | 14: 23217984 | 67184 | 14: 23219378 | 67185 | 14: 23220306 | 67186 | 14: 23221060 | 67187 | 14: 23221105 |
| 67188 | 14: 23221660 | 67190 | 14: 23223448 | 67192 | 14: 23232920 | 67293 | 14: 23296927 | 67353 | 14: 23590382 |
| 67532 | 14: 25538462 | 67551 | 14: 25555299 | 67553 | 14: 25557838 | 67562 | 14: 25875465 | 67572 | 14: 26025356 |
| 67592 | 14: 26183095 | 67615 | 14: 26313111 | 67631 | 14: 26716130 | 67638 | 14: 26937569 | 67647 | 14: 27145363 |
| 67659 | 14: 27578987 | 67666 | 14: 27804049 | 67669 | 14: 27925661 | 67680 | 14: 28283881 | 67684 | 14: 28475888 |
| 67701 | 14: 28805553 | 67713 | 14: 29204640 | 67728 | 14: 29506987 | 67737 | 14: 29651348 | 67751 | 14: 30011410 |
| 67787 | 14: 30531165 | 67806 | 14: 31151661 | 67810 | 14: 31229071 | 67812 | 14: 31276668 | 67813 | 14: 31348707 |
| 67839 | 14: 32042320 | 67840 | 14: 32080892 | 67847 | 14: 32295836 | 67848 | 14: 32298088 | 67849 | 14: 32298121 |
| 67850 | 14: 32298404 | 67851 | 14: 32298532 | 67852 | 14: 32298550 | 67853 | 14: 32298592 | 67854 | 14: 32298623 |
| 67855 | 14: 32298715 | 67856 | 14: 32298807 | 67857 | 14: 32298889 | 67858 | 14: 32298929 | 67859 | 14: 32298949 |
| 67860 | 14: 32298983 | 67861 | 14: 32301242 | 67862 | 14: 32301629 | 67863 | 14: 32301804 | 67880 | 14: 32768091 |
| 67902 | 14: 33283287 | 67914 | 14: 33481134 | 67945 | 14: 34107991 | 67963 | 14: 34616797 | 67966 | 14: 34703575 |
| 67968 | 14: 34726901 | 68040 | 14: 36456652 | 68066 | 14: 37335063 | 68131 | 14: 39204823 | 68180 | 14: 40564678 |
| 68185 | 14: 40761575 | 68199 | 14: 41245865 | 68207 | 14: 41477789 | 68211 | 14: 41608564 | 68246 | 14: 42371257 |
| 68252 | 14: 42499551 | 68275 | 14: 43217965 | 68314 | 14: 44597633 | 68367 | 14: 45598918 | 68386 | 14: 45953548 |
| 68393 | 14: 46043440 | 68412 | 14: 46302755 | 68430 | 14: 46746014 | 68435 | 14: 46846924 | 68463 | 14: 47562082 |
| 68479 | 14: 48154116 | 68495 | 14: 48422440 | 68496 | 14: 48424765 | 68507 | 14: 48722429 | 68578 | 14: 50964252 |
| 68611 | 14: 51741282 | 68659 | 14: 53067901 | 68716 | 14: 54352030 | 68717 | 14: 54359397 | 68727 | 14: 54470262 |
| 68733 | 14: 54629600 | 68785 | 14: 56030241 | 68869 | 14: 58169542 | 68876 | 14: 58268988 | 68879 | 14: 58347367 |
| 68898 | 14: 58795728 | 68902 | 14: 58882362 | 68914 | 14: 59072940 | 68931 | 14: 59422421 | 68945 | 14: 59890123 |
| 68960 | 14: 60186718 | 68998 | 14: 61242862 | 69030 | 14: 61805601 | 69069 | 14: 62451453 | 69075 | 14: 62584372 |
| 69079 | 14: 62700380 | 69092 | 14: 62957699 | 69106 | 14: 63240134 | 69117 | 14: 63473862 | 69160 | 14: 64610567 |
| 69174 | 14: 65146328 | 69215 | 14: 66160998 | 69219 | 14: 66259689 | 69228 | 14: 66384994 | 69232 | 14: 66419660 |
| 69326 | 14: 68515393 | 69354 | 14: 69351733 | 69370 | 14: 69913790 | 69382 | 14: 70082152 | 69391 | 14: 70201845 |
| 69400 | 14: 70384514 | 69408 | 14: 70595169 | 69415 | 14: 70881552 | 69416 | 14: 70890232 | 69427 | 14: 71123353 |
| 69443 | 14: 71687136 | 69472 | 14: 72410713 | 69494 | 14: 72897923 | 69506 | 14: 73212493 | 69515 | 14: 73427877 |
| 69517 | 14: 73468390 | 69534 | 14: 73985016 | 69570 | 14: 75029436 | 69571 | 14: 75034802 | 69572 | 14: 75035607 |
| 69573 | 14: 75035827 | 69574 | 14: 75035857 | 69575 | 14: 75035923 | 69608 | 14: 76029716 | 69631 | 14: 76258289 |
| 69633 | 14: 76268094 | 69680 | 14: 77464768 | 69686 | 14: 77542458 | 69690 | 14: 77708662 | 69700 | 14: 77954720 |
| 69736 | 14: 78637209 | 69737 | 14: 78644200 | 69742 | 14: 78699024 | 69750 | 14: 78929919 | 69762 | 14: 79335372 |
| 69767 | 14: 79486373 | 69783 | 14: 79820967 | 69790 | 14: 79974645 | 69805 | 14: 80236030 | 69819 | 14: 80591048 |
| 69864 | 14: 81465207 | 69901 | 14: 82003511 | 70000 | 15: 2555400 | 70037 | 15: 3651481 | 70043 | 15: 3796804 |
| 70044 | 15: 3859257 | 70045 | 15: 3861053 | 70046 | 15: 3861177 | 70048 | 15: 3872949 | 70049 | 15: 3874148 |
| 70050 | 15: 3874413 | 70051 | 15: 3875123 | 70052 | 15: 3875291 | 70053 | 15: 3875480 | 70054 | 15: 3875800 |
| 70055 | 15: 3876161 | 70056 | 15: 3876211 | 70057 | 15: 3876259 | 70058 | 15: 3884992 | 70059 | 15: 3885455 |
| 70060 | 15: 3888242 | 70061 | 15: 3888270 | 70070 | 15: 4006347 | 70080 | 15: 4274305 | 70107 | 15: 4817619 |
| 70108 | 15: 4821833 | 70109 | 15: 4827554 | 70110 | 15: 4850527 | 70111 | 15: 4855973 | 70113 | 15: 4859691 |
| 70114 | 15: 4874530 | 70132 | 15: 5345428 | 70133 | 15: 5345893 | 70134 | 15: 5346513 | 70135 | 15: 5346544 |
| 70136 | 15: 5347645 | 70137 | 15: 5348421 | 70138 | 15: 5348505 | 70139 | 15: 5348800 | 70140 | 15: 5348852 |
| 70141 | 15: 5348875 | 70142 | 15: 5349143 | 70143 | 15: 5349849 | 70144 | 15: 5350256 | 70145 | 15: 5350323 |
| 70146 | 15: 5350355 | 70147 | 15: 5353059 | 70148 | 15: 5353099 | 70149 | 15: 5355811 | 70150 | 15: 5355909 |
| 70151 | 15: 5356157 | 70152 | 15: 5356186 | 70153 | 15: 5356365 | 70154 | 15: 5356372 | 70155 | 15: 5357215 |
| 70156 | 15: 5358118 | 70157 | 15: 5358230 | 70158 | 15: 5358668 | 70159 | 15: 5359593 | 70160 | 15: 5359865 |
| 70161 | 15: 5359868 | 70162 | 15: 5361660 | 70163 | 15: 5361661 | 70164 | 15: 5365576 | 70165 | 15: 5380375 |
| 70166 | 15: 5381250 | 70167 | 15: 5382096 | 70168 | 15: 5383165 | 70169 | 15: 5383541 | 70170 | 15: 5384183 |
| 70171 | 15: 5384848 | 70172 | 15: 5384963 | 70173 | 15: 5384997 | 70174 | 15: 5385062 | 70175 | 15: 5385270 |
| 70176 | 15: 5385290 | 70177 | 15: 5385312 | 70178 | 15: 5385429 | 70179 | 15: 5386627 | 70180 | 15: 5386633 |
| 70181 | 15: 5386655 | 70182 | 15: 5386721 | 70183 | 15: 5387773 | 70184 | 15: 5387850 | 70185 | 15: 5388162 |
| 70186 | 15: 5388165 | 70187 | 15: 5388635 | 70188 | 15: 5388661 | 70189 | 15: 5388748 | 70190 | 15: 5389424 |
| 70191 | 15: 5389588 | 70192 | 15: 5389616 | 70193 | 15: 5389859 | 70194 | 15: 5389978 | 70195 | 15: 5390296 |
| 70196 | 15: 5390320 | 70197 | 15: 5390450 | 70198 | 15: 5390553 | 70199 | 15: 5390863 | 70200 | 15: 5390921 |
| 70201 | 15: 5391192 | 70202 | 15: 5391298 | 70203 | 15: 5391508 | 70204 | 15: 5391682 | 70205 | 15: 5391799 |
| 70206 | 15: 5392452 | 70207 | 15: 5392859 | 70208 | 15: 5392923 | 70209 | 15: 5393194 | 70210 | 15: 5393401 |
| 70211 | 15: 5393671 | 70212 | 15: 5393753 | 70213 | 15: 5394283 | 70214 | 15: 5395547 | 70215 | 15: 5395610 |
| 70216 | 15: 5395743 | 70217 | 15: 5395751 | 70218 | 15: 5395855 | 70219 | 15: 5395947 | 70220 | 15: 5398109 |
| 70221 | 15: 5398341 | 70222 | 15: 5398346 | 70223 | 15: 5398513 | 70224 | 15: 5398559 | 70225 | 15: 5400626 |
| 70226 | 15: 5401120 | 70227 | 15: 5401525 | 70228 | 15: 5401536 | 70229 | 15: 5401568 | 70230 | 15: 5401820 |
| 70231 | 15: 5401853 | 70232 | 15: 5404746 | 70233 | 15: 5404825 | 70234 | 15: 5405498 | 70235 | 15: 5405505 |
| 70236 | 15: 5406067 | 70237 | 15: 5406105 | 70238 | 15: 5406213 | 70239 | 15: 5406470 | 70240 | 15: 5406961 |
| 70241 | 15: 5407075 | 70242 | 15: 5407142 | 70243 | 15: 5407235 | 70244 | 15: 5407350 | 70245 | 15: 5407539 |
| 70246 | 15: 5407760 | 70247 | 15: 5407841 | 70248 | 15: 5407895 | 70249 | 15: 5408434 | 70250 | 15: 5408552 |
| 70251 | 15: 5408640 | 70252 | 15: 5408900 | 70253 | 15: 5409527 | 70254 | 15: 5409532 | 70255 | 15: 5409637 |
| 70256 | 15: 5409728 | 70257 | 15: 5409914 | 70258 | 15: 5410082 | 70259 | 15: 5410166 | 70260 | 15: 5410173 |
| 70261 | 15: 5410337 | 70262 | 15: 5410342 | 70263 | 15: 5410429 | 70264 | 15: 5410813 | 70265 | 15: 5410848 |
| 70266 | 15: 5412771 | 70267 | 15: 5412796 | 70268 | 15: 5412835 | 70269 | 15: 5412869 | 70270 | 15: 5413090 |
| 70271 | 15: 5413111 | 70272 | 15: 5413650 | 70273 | 15: 5415034 | 70274 | 15: 5415477 | 70275 | 15: 5416298 |
| 70276 | 15: 5416324 | 70277 | 15: 5417129 | 70278 | 15: 5417450 | 70279 | 15: 5417663 | 70280 | 15: 5418542 |
| 70284 | 15: 5503948 | 70303 | 15: 6004286 | 70319 | 15: 6435496 | 70337 | 15: 7003211 | 70338 | 15: 7008452 |
| 70340 | 15: 7019231 | 70341 | 15: 7029657 | 70342 | 15: 7033643 | 70348 | 15: 7265163 | 70349 | 15: 7265165 |
| 70350 | 15: 7265341 | 70351 | 15: 7265425 | 70352 | 15: 7265838 | 70353 | 15: 7270627 | 70354 | 15: 7270650 |
| 70355 | 15: 7272155 | 70356 | 15: 7272347 | 70357 | 15: 7272651 | 70358 | 15: 7275537 | 70359 | 15: 7275789 |
| 70360 | 15: 7275947 | 70361 | 15: 7276254 | 70362 | 15: 7277640 | 70363 | 15: 7277661 | 70364 | 15: 7278217 |
| 70411 | 15: 8492046 | 70427 | 15: 9014277 | 70429 | 15: 9031126 | 70432 | 15: 9102618 | 70438 | 15: 9169770 |
| 70439 | 15: 9233443 | 70471 | 15: 10068806 | 70472 | 15: 10075553 | 70474 | 15: 10079990 | 70475 | 15: 10080765 |
| 70476 | 15: 10081644 | 70511 | 15: 11396612 | 70557 | 15: 13243732 | 70596 | 15: 14363124 | 70609 | 15: 14575841 |
| 70610 | 15: 14594338 | 70629 | 15: 15205635 | 70630 | 15: 15239992 | 70707 | 15: 16376589 | 70708 | 15: 16439066 |
| 70735 | 15: 17085252 | 70738 | 15: 17150531 | 70745 | 15: 17184652 | 70765 | 15: 17609663 | 70797 | 15: 18443747 |
| 70805 | 15: 18738117 | 70819 | 15: 19107499 | 70839 | 15: 19655124 | 70859 | 15: 20139691 | 70869 | 15: 20440646 |
| 70890 | 15: 20723501 | 70945 | 15: 22143335 | 70975 | 15: 22778474 | 70976 | 15: 22778563 | 70977 | 15: 22779768 |
| 70984 | 15: 22928839 | 70990 | 15: 23127718 | 70993 | 15: 23186412 | 71073 | 15: 24244322 | 71098 | 15: 24733535 |
| 71100 | 15: 24758009 | 71125 | 15: 25277078 | 71134 | 15: 25472864 | 71147 | 15: 25791312 | 71158 | 15: 25955785 |
| 71179 | 15: 26356076 | 71196 | 15: 26746546 | 71208 | 15: 27116571 | 71241 | 15: 27939885 | 71250 | 15: 28057977 |
| 71255 | 15: 28119260 | 71281 | 15: 28579106 | 71361 | 15: 30204684 | 71411 | 15: 31423842 | 71480 | 15: 32908026 |
| 71484 | 15: 32949060 | 71520 | 15: 33787224 | 71547 | 15: 34234555 | 71557 | 15: 34409813 | 71575 | 15: 34768501 |
| 71595 | 15: 35125952 | 71629 | 15: 36004891 | 71630 | 15: 36008307 | 71631 | 15: 36026492 | 71648 | 15: 36401889 |
| 71659 | 15: 36632711 | 71691 | 15: 37336930 | 71694 | 15: 37400581 | 71695 | 15: 37400733 | 71696 | 15: 37402318 |
| 71697 | 15: 37402363 | 71698 | 15: 37402682 | 71699 | 15: 37403163 | 71700 | 15: 37405162 | 71701 | 15: 37406604 |
| 71702 | 15: 37408508 | 71726 | 15: 38124244 | 71729 | 15: 38238774 | 71747 | 15: 38627437 | 71754 | 15: 38756627 |
| 71765 | 15: 39027306 | 71774 | 15: 39310122 | 71779 | 15: 39360198 | 71788 | 15: 39612413 | 71831 | 15: 40620626 |
| 71848 | 15: 41008572 | 71872 | 15: 41577758 | 71873 | 15: 41586379 | 71894 | 15: 41925488 | 71937 | 15: 42825209 |
| 71945 | 15: 42913923 | 71990 | 15: 43478979 | 72010 | 15: 43778894 | 72015 | 15: 43842403 | 72025 | 15: 43940904 |
| 72031 | 15: 44171870 | 72055 | 15: 44625080 | 72119 | 15: 46158689 | 72132 | 15: 46492762 | 72164 | 15: 46744389 |
| 72165 | 15: 46777715 | 72167 | 15: 46785999 | 72168 | 15: 46790581 | 72169 | 15: 46804156 | 72170 | 15: 46852906 |
| 72171 | 15: 46854891 | 72175 | 15: 46873315 | 72219 | 15: 47951518 | 72223 | 15: 48016378 | 72242 | 15: 48211767 |
| 72259 | 15: 48584212 | 72288 | 15: 49705651 | 72400 | 15: 52067358 | 72432 | 15: 52906987 | 72450 | 15: 53308730 |
| 72455 | 15: 53312462 | 72514 | 15: 54171179 | 72571 | 15: 55496596 | 72580 | 15: 55679447 | 72584 | 15: 55794756 |
| 72629 | 15: 56617358 | 72704 | 15: 57721772 | 72710 | 15: 57822336 | 72753 | 15: 58596845 | 72786 | 15: 59497090 |
| 72810 | 15: 60299723 | 72821 | 15: 60544990 | 72828 | 15: 60665356 | 72838 | 15: 61116189 | 72841 | 15: 61144336 |
| 72844 | 15: 61187610 | 72847 | 15: 61230749 | 72848 | 15: 61235200 | 72883 | 15: 61807140 | 72897 | 15: 62087105 |
| 72904 | 15: 62399301 | 72913 | 15: 62562520 | 72945 | 15: 63169519 | 72956 | 15: 63484094 | 72978 | 15: 63866432 |
| 72980 | 15: 63956384 | 72984 | 15: 63969563 | 73010 | 15: 64570035 | 73011 | 15: 64573599 | 73012 | 15: 64581068 |
| 73013 | 15: 64581832 | 73014 | 15: 64581860 | 73015 | 15: 64587577 | 73016 | 15: 64587694 | 73017 | 15: 64587726 |
| 73018 | 15: 64587727 | 73019 | 15: 64587894 | 73032 | 15: 64805974 | 73034 | 15: 64822114 | 73036 | 15: 64828957 |
| 73037 | 15: 64829763 | 73055 | 15: 65133696 | 73057 | 15: 65176452 | 73068 | 15: 65338322 | 73110 | 15: 66198995 |
| 73133 | 15: 66626921 | 73136 | 15: 66654107 | 73137 | 15: 66659219 | 73157 | 15: 67252520 | 73183 | 15: 67982185 |
| 73232 | 15: 69401513 | 73297 | 15: 71439330 | 73316 | 15: 71924774 | 73318 | 15: 71954992 | 73320 | 15: 71975292 |
| 73324 | 15: 72049215 | 73432 | 15: 74395188 | 73433 | 15: 74409395 | 73453 | 15: 74803075 | 73462 | 15: 75064377 |
| 73463 | 15: 75065891 | 73464 | 15: 75071311 | 73469 | 15: 75183095 | 73471 | 15: 75207042 | 73478 | 15: 75433003 |
| 73581 | 15: 77653525 | 73585 | 15: 77702400 | 73588 | 15: 77785862 | 73593 | 15: 77886217 | 73647 | 15: 78764970 |
| 73658 | 15: 79089495 | 73691 | 15: 80200231 | 73697 | 15: 80308461 | 73708 | 15: 80425465 | 73780 | 15: 81982872 |
| 73833 | 15: 83017252 | 73857 | 15: 83501029 | 73858 | 15: 83507518 | 73859 | 15: 83514913 | 73860 | 15: 83519848 |
| 73861 | 15: 83524365 | 73863 | 15: 83529608 | 73885 | 15: 84090905 | 73971 | 16: 1756551 | 74018 | 16: 2807132 |
| 74022 | 16: 2880813 | 74046 | 16: 3278886 | 74067 | 16: 3645905 | 74074 | 16: 3832226 | 74078 | 16: 3983617 |
| 74085 | 16: 4041201 | 74113 | 16: 4472306 | 74116 | 16: 4488724 | 74134 | 16: 5011968 | 74222 | 16: 7875660 |
| 74227 | 16: 8061836 | 74247 | 16: 8833166 | 74264 | 16: 9480615 | 74299 | 16: 10761697 | 74367 | 16: 12564264 |
| 74370 | 16: 12610513 | 74376 | 16: 12712264 | 74380 | 16: 12809176 | 74416 | 16: 13500639 | 74493 | 16: 15596092 |
| 74509 | 16: 16114051 | 74543 | 16: 17341478 | 74577 | 16: 18497037 | 74680 | 16: 21073857 | 74697 | 16: 21482215 |
| 74705 | 16: 21676658 | 74716 | 16: 22006522 | 74717 | 16: 22052486 | 74721 | 16: 22158028 | 74731 | 16: 22445521 |
| 74735 | 16: 22522151 | 74736 | 16: 22527368 | 74784 | 16: 23721556 | 74795 | 16: 23949095 | 74810 | 16: 24332233 |
| 74873 | 16: 25194415 | 74886 | 16: 25230576 | 74895 | 16: 25259731 | 74896 | 16: 25267299 | 74912 | 16: 25869035 |
| 74914 | 16: 25919271 | 74922 | 16: 26068625 | 74962 | 16: 26927299 | 75005 | 16: 28019627 | 75093 | 16: 30731403 |
| 75115 | 16: 31224822 | 75116 | 16: 31245895 | 75135 | 16: 31715125 | 75141 | 16: 31812156 | 75192 | 16: 33108294 |
| 75193 | 16: 33145311 | 75196 | 16: 33256074 | 75208 | 16: 33505042 | 75221 | 16: 33889094 | 75227 | 16: 34042467 |
| 75228 | 16: 34043548 | 75232 | 16: 34106781 | 75248 | 16: 34589155 | 75329 | 16: 36525688 | 75352 | 16: 37160322 |
| 75435 | 16: 39120749 | 75447 | 16: 39536783 | 75474 | 16: 40415228 | 75484 | 16: 40686277 | 75519 | 16: 41883664 |
| 75545 | 16: 42004893 | 75548 | 16: 42055479 | 75562 | 16: 42802530 | 75565 | 16: 42843655 | 75640 | 16: 46097209 |
| 75644 | 16: 46156095 | 75677 | 16: 46962612 | 75715 | 16: 47752936 | 75724 | 16: 48024037 | 75725 | 16: 48025956 |
| 75726 | 16: 48033049 | 75727 | 16: 48034263 | 75729 | 16: 48067867 | 75734 | 16: 48186793 | 75735 | 16: 48187331 |
| 75736 | 16: 48187489 | 75737 | 16: 48223403 | 75738 | 16: 48230117 | 75739 | 16: 48232464 | 75751 | 16: 48528203 |
| 75754 | 16: 48566545 | 75755 | 16: 48591914 | 75771 | 16: 48864424 | 75776 | 16: 49014011 | 75786 | 16: 49264493 |
| 75852 | 16: 51207748 | 75916 | 16: 52579557 | 76051 | 16: 56128576 | 76058 | 16: 56235648 | 76083 | 16: 56653049 |
| 76110 | 16: 57538081 | 76150 | 16: 58399171 | 76164 | 16: 58621735 | 76178 | 16: 58963242 | 76182 | 16: 59073877 |
| 76184 | 16: 59075986 | 76190 | 16: 59245993 | 76192 | 16: 59265813 | 76193 | 16: 59266170 | 76200 | 16: 59328729 |
| 76205 | 16: 59411723 | 76226 | 16: 60040160 | 76243 | 16: 60336822 | 76259 | 16: 60792834 | 76289 | 16: 61435887 |
| 76345 | 16: 62739421 | 76365 | 16: 63159271 | 76368 | 16: 63219362 | 76383 | 16: 63498892 | 76385 | 16: 63554652 |
| 76398 | 16: 63842771 | 76400 | 16: 63858162 | 76411 | 16: 64136818 | 76424 | 16: 64408574 | 76435 | 16: 64563350 |
| 76440 | 16: 64632519 | 76442 | 16: 64637819 | 76443 | 16: 64637824 | 76444 | 16: 64637826 | 76453 | 16: 64811600 |
| 76524 | 16: 66273725 | 76529 | 16: 66428578 | 76542 | 16: 66625242 | 76552 | 16: 66834448 | 76585 | 16: 67467822 |
| 76587 | 16: 67495360 | 76590 | 16: 67506416 | 76593 | 16: 67552727 | 76603 | 16: 67685441 | 76635 | 16: 68623796 |
| 76640 | 16: 68718917 | 76642 | 16: 68746747 | 76646 | 16: 68890172 | 76655 | 16: 69079413 | 76691 | 16: 69777086 |
| 76693 | 16: 69813037 | 76701 | 16: 70045391 | 76717 | 16: 70339833 | 76729 | 16: 70576773 | 76748 | 16: 70946593 |
| 76774 | 16: 71760780 | 76788 | 16: 72102560 | 76825 | 16: 72662418 | 76830 | 16: 72687591 | 76842 | 16: 72970330 |
| 76861 | 16: 73473194 | 76864 | 16: 73513959 | 76895 | 16: 73873564 | 76951 | 16: 75067479 | 76972 | 16: 75350380 |
| 76990 | 16: 75714163 | 77032 | 16: 76538771 | 77064 | 16: 77153817 | 77099 | 16: 77965851 | 77102 | 16: 78019437 |
| 77132 | 16: 78710736 | 77152 | 16: 78992583 | 77166 | 16: 79102626 | 77238 | 16: 80715002 | 77241 | 16: 80886197 |
| 77246 | 17: 111106 | 77250 | 17: 171222 | 77251 | 17: 172400 | 77253 | 17: 305126 | 77262 | 17: 486383 |
| 77374 | 17: 3335273 | 77379 | 17: 3441488 | 77422 | 17: 4260319 | 77443 | 17: 4584704 | 77446 | 17: 4621398 |
| 77452 | 17: 4717447 | 77469 | 17: 5097696 | 77470 | 17: 5097857 | 77489 | 17: 5622055 | 77510 | 17: 6077823 |
| 77557 | 17: 6872378 | 77579 | 17: 7317589 | 77580 | 17: 7338578 | 77582 | 17: 7342145 | 77586 | 17: 7478372 |
| 77606 | 17: 7876357 | 77608 | 17: 7883935 | 77619 | 17: 8132536 | 77638 | 17: 8600944 | 77642 | 17: 8668787 |
| 77704 | 17: 10109146 | 77716 | 17: 10263523 | 77725 | 17: 10597515 | 77742 | 17: 11173860 | 77747 | 17: 11293045 |
| 77753 | 17: 11395090 | 77762 | 17: 11714108 | 77763 | 17: 11715058 | 77770 | 17: 11817054 | 77787 | 17: 12195499 |
| 77788 | 17: 12195568 | 77789 | 17: 12195662 | 77790 | 17: 12196276 | 77791 | 17: 12196708 | 77805 | 17: 12486159 |
| 77808 | 17: 12628635 | 77906 | 17: 14816935 | 77909 | 17: 14863506 | 77931 | 17: 15328888 | 77949 | 17: 15663502 |
| 77958 | 17: 15910083 | 77975 | 17: 16368625 | 78009 | 17: 17274244 | 78010 | 17: 17279868 | 78011 | 17: 17280788 |
| 78012 | 17: 17280990 | 78013 | 17: 17281225 | 78024 | 17: 17411908 | 78039 | 17: 17775173 | 78082 | 17: 18818243 |
| 78086 | 17: 18869685 | 78105 | 17: 19223651 | 78106 | 17: 19245275 | 78109 | 17: 19292348 | 78110 | 17: 19295355 |
| 78111 | 17: 19295356 | 78112 | 17: 19295499 | 78113 | 17: 19295504 | 78133 | 17: 19689334 | 78153 | 17: 20218072 |
| 78173 | 17: 20920601 | 78177 | 17: 21089322 | 78195 | 17: 21650017 | 78203 | 17: 21824364 | 78259 | 17: 24078894 |
| 78278 | 17: 24557966 | 78308 | 17: 25485693 | 78320 | 17: 25835294 | 78333 | 17: 26208169 | 78336 | 17: 26275689 |
| 78338 | 17: 26313864 | 78347 | 17: 26586857 | 78355 | 17: 26669513 | 78379 | 17: 27150917 | 78417 | 17: 28123761 |
| 78429 | 17: 28336908 | 78457 | 17: 29248479 | 78514 | 17: 30796206 | 78543 | 17: 31545528 | 78556 | 17: 31865010 |
| 78558 | 17: 31877966 | 78579 | 17: 32478146 | 78583 | 17: 32532692 | 78596 | 17: 32813643 | 78625 | 17: 33559323 |
| 78633 | 17: 33630931 | 78661 | 17: 34398154 | 78696 | 17: 35542521 | 78722 | 17: 36254116 | 78747 | 17: 37178207 |
| 78773 | 17: 38080956 | 78783 | 17: 38278802 | 7878 | 17: 38562984 | 78797 | 17: 38814783 | 78824 | 17: 39676479 |
| 78825 | 17: 39677255 | 78826 | 17: 39679094 | 78832 | 17: 39827471 | 78833 | 17: 39828115 | 78834 | 17: 39828525 |
| 78836 | 17: 39840626 | 78838 | 17: 39849566 | 78839 | 17: 39854144 | 78840 | 17: 39858951 | 78843 | 17: 39882893 |
| 78844 | 17: 39883222 | 78845 | 17: 39885556 | 78846 | 17: 39886164 | 78847 | 17: 39887756 | 78848 | 17: 39893701 |
| 78849 | 17: 39894975 | 78850 | 17: 39896327 | 78851 | 17: 39896972 | 78877 | 17: 40559838 | 78880 | 17: 40569673 |
| 78881 | 17: 40569857 | 78882 | 17: 40570434 | 78885 | 17: 40597210 | 78889 | 17: 40687599 | 78891 | 17: 40702874 |
| 78893 | 17: 40727620 | 78924 | 17: 41582600 | 78993 | 17: 43272612 | 78996 | 17: 43327605 | 79034 | 17: 44279469 |
| 79079 | 17: 45388796 | 79090 | 17: 45567473 | 79117 | 17: 46182697 | 79173 | 17: 47495174 | 79183 | 17: 47822968 |
| 79184 | 17: 47826553 | 79203 | 17: 48325513 | 79216 | 17: 48653000 | 79227 | 17: 48892420 | 79233 | 17: 49128208 |
| 79234 | 17: 49131387 | 79272 | 17: 49921719 | 79309 | 17: 50714394 | 79310 | 17: 50720459 | 79311 | 17: 50722838 |
| 79312 | 17: 50723471 | 79313 | 17: 50727878 | 79314 | 17: 50730200 | 79315 | 17: 50731825 | 79317 | 17: 50814958 |
| 79357 | 17: 51667657 | 79381 | 17: 52060992 | 79385 | 17: 52081616 | 79386 | 17: 52082865 | 79387 | 17: 52087837 |
| 79388 | 17: 52087901 | 79424 | 17: 53278122 | 79462 | 17: 54135006 | 79538 | 17: 56024288 | 79550 | 17: 56299499 |
| 79555 | 17: 56435818 | 79572 | 17: 57052924 | 79586 | 17: 57342574 | 79605 | 17: 57757425 | 79607 | 17: 57774071 |
| 79611 | 17: 57814505 | 79628 | 17: 58146798 | 79636 | 17: 58314278 | 79637 | 17: 58317198 | 79638 | 17: 58321497 |
| 79639 | 17: 58322279 | 79640 | 17: 58323397 | 79641 | 17: 58324863 | 79642 | 17: 58325379 | 79643 | 17: 58325583 |
| 79644 | 17: 58325642 | 79645 | 17: 58325662 | 79646 | 17: 58325692 | 79647 | 17: 58325868 | 79648 | 17: 58325926 |
| 79649 | 17: 58326148 | 79650 | 17: 58330227 | 79651 | 17: 58331401 | 79653 | 17: 58334167 | 79654 | 17: 58335083 |
| 79655 | 17: 58335597 | 79656 | 17: 58337576 | 79657 | 17: 58338335 | 79681 | 17: 59019311 | 79699 | 17: 59378547 |
| 79701 | 17: 59392366 | 79712 | 17: 59683258 | 79747 | 17: 60329105 | 79749 | 17: 60358707 | 79757 | 17: 60444808 |
| 79798 | 17: 61041921 | 79820 | 17: 61336784 | 79824 | 17: 61432513 | 79831 | 17: 61511820 | 79893 | 17: 63198489 |
| 79977 | 17: 64661316 | 79989 | 17: 64973779 | 79994 | 17: 65038744 | 80026 | 17: 65697499 | 80041 | 17: 65972365 |
| 80051 | 17: 66165602 | 80052 | 17: 66169310 | 80053 | 17: 66170354 | 80054 | 17: 66171241 | 80055 | 17: 66171742 |
| 80056 | 17: 66172830 | 80071 | 17: 66374177 | 80113 | 17: 67428489 | 80128 | 17: 67720645 | 80131 | 17: 67842378 |
| 80139 | 17: 68113867 | 80189 | 17: 69059007 | 80213 | 17: 69580013 | 80223 | 17: 69757640 | 80227 | 17: 69882555 |
| 80246 | 17: 70318401 | 80276 | 17: 70659081 | 80282 | 17: 70929780 | 80318 | 17: 71539730 | 80327 | 17: 71761848 |
| 80331 | 17: 71853596 | 80332 | 17: 71855169 | 80333 | 17: 71859405 | 80335 | 17: 71900536 | 80406 | 18: 712517 |
| 80420 | 18: 990363 | 80449 | 18: 1484040 | 80458 | 18: 1637197 | 80473 | 18: 1743436 | 80501 | 18: 1802242 |
| 80511 | 18: 2001978 | 80533 | 18: 2557098 | 80550 | 18: 2832635 | 80557 | 18: 2935768 | 80565 | 18: 3102978 |
| 80575 | 18: 3297749 | 80603 | 18: 4036722 | 80612 | 18: 4219952 | 80613 | 18: 4226405 | 80620 | 18: 4439946 |
| 80628 | 18: 4565913 | 80644 | 18: 4871817 | 80669 | 18: 5415676 | 80670 | 18: 5415711 | 80671 | 18: 5416566 |
| 80712 | 18: 6220320 | 80723 | 18: 6398640 | 80725 | 18: 6407051 | 80726 | 18: 6407842 | 80727 | 18: 6407856 |
| 80728 | 18: 6408160 | 80729 | 18: 6409137 | 80730 | 18: 6409528 | 80748 | 18: 6759745 | 80753 | 18: 6859408 |
| 80770 | 18: 7273399 | 80793 | 18: 7769343 | 80802 | 18: 7953745 | 80851 | 18: 8931926 | 80872 | 18: 9156689 |
| 80884 | 18: 9413940 | 80885 | 18: 9414216 | 80909 | 18: 9827683 | 80924 | 18: 10096838 | 80935 | 18: 10422385 |
| 80980 | 18: 11195149 | 81051 | 18: 13077929 | 81083 | 18: 13931354 | 81085 | 18: 13962063 | 81086 | 18: 13962211 |
| 81094 | 18: 14131401 | 81128 | 18: 15337353 | 81175 | 18: 16420426 | 81195 | 18: 16937183 | 81202 | 18: 17081163 |
| 81213 | 18: 17191494 | 81240 | 18: 17819065 | 81249 | 18: 18041884 | 81257 | 18: 18196329 | 81262 | 18: 18274003 |
| 81263 | 18: 18274911 | 81264 | 18: 18279929 | 81265 | 18: 18284551 | 81266 | 18: 18285334 | 81268 | 18: 18287100 |
| 81270 | 18: 18297472 | 81271 | 18: 18300144 | 81289 | 18: 18654230 | 81301 | 18: 18883470 | 81336 | 18: 19805409 |
| 81365 | 18: 20391312 | 81369 | 18: 20532028 | 81370 | 18: 20581067 | 81371 | 18: 20586213 | 81372 | 18: 20591204 |
| 81373 | 18: 20594221 | 81383 | 18: 20851094 | 81434 | 18: 22045866 | 81499 | 18: 23267678 | 81519 | 18: 23730169 |
| 81533 | 18: 24068173 | 81584 | 18: 25002158 | 81600 | 18: 25402965 | 81616 | 18: 25722314 | 81657 | 18: 26620395 |
| 81660 | 18: 26643719 | 81667 | 18: 26781973 | 81699 | 18: 27670793 | 81722 | 18: 28212436 | 81754 | 18: 29332445 |
| 81762 | 18: 29394581 | 81775 | 18: 29615496 | 81779 | 18: 29751573 | 81780 | 18: 29770581 | 81825 | 18: 31291350 |
| 81850 | 18: 31971311 | 81863 | 18: 32267474 | 81869 | 18: 32301839 | 81874 | 18: 32426539 | 81917 | 18: 33371652 |
| 81918 | 18: 33381630 | 81919 | 18: 33381846 | 81921 | 18: 33389064 | 81930 | 18: 33546416 | 81931 | 18: 33559158 |
| 81956 | 18: 34226859 | 82000 | 18: 35324050 | 82017 | 18: 35722943 | 82090 | 18: 37440845 | 82096 | 18: 37511943 |
| 82111 | 18: 37857222 | 82117 | 18: 37902748 | 82162 | 18: 38995251 | 82167 | 18: 39072678 | 82201 | 18: 39856656 |
| 82211 | 18: 40078593 | 82214 | 18: 40135404 | 82268 | 18: 41177791 | 82300 | 18: 41952487 | 82332 | 18: 42484052 |
| 82374 | 18: 43224250 | 82381 | 18: 43359712 | 82384 | 18: 43378123 | 82402 | 18: 43762058 | 82434 | 18: 44688799 |
| 82436 | 18: 44703246 | 82492 | 18: 46177257 | 82495 | 18: 46269611 | 82566 | 18: 46759650 | 82608 | 18: 47374533 |
| 82675 | 18: 48506540 | 82731 | 18: 49938198 | 82779 | 18: 51259647 | 82786 | 18: 51451371 | 82787 | 18: 51452185 |
| 82788 | 18: 51455635 | 82792 | 18: 51522776 | 82795 | 18: 51579528 | 82817 | 18: 51820303 | 82847 | 18: 52482506 |
| 82860 | 18: 52950856 | 82873 | 18: 53350033 | 82882 | 18: 53554813 | 82896 | 18: 53718739 | 82916 | 18: 54039068 |
| 82934 | 18: 54414979 | 82973 | 18: 55042223 | 83009 | 18: 55891884 | 83050 | 18: 56619470 | 83056 | 18: 56702388 |
| 83061 | 18: 56777892 | 83063 | 18: 56780574 | 83064 | 18: 56781239 | 83065 | 18: 56788137 | 83066 | 18: 56788537 |
| 83067 | 18: 56790131 | 83068 | 18: 56793472 | 83069 | 18: 56793653 | 83070 | 18: 56802310 | 83071 | 18: 56802366 |
| 83072 | 18: 56802408 | 83073 | 18: 56803155 | 83074 | 18: 56805465 | 83075 | 18: 56805669 | 83076 | 18: 56805763 |
| 83077 | 18: 56805884 | 83078 | 18: 56805908 | 83079 | 18: 56805969 | 83080 | 18: 56806452 | 83081 | 18: 56806522 |
| 83082 | 18: 56806817 | 83083 | 18: 56807069 | 83084 | 18: 56807143 | 83085 | 18: 56807200 | 83086 | 18: 56808520 |
| 83087 | 18: 56808693 | 83088 | 18: 56808739 | 83089 | 18: 56809175 | 83090 | 18: 56809229 | 83091 | 18: 56809274 |
| 83092 | 18: 56809314 | 83093 | 18: 56809399 | 83094 | 18: 56811400 | 83095 | 18: 56816836 | 83096 | 18: 56817529 |
| 83097 | 18: 56817530 | 83098 | 18: 56817556 | 83099 | 18: 56817983 | 83100 | 18: 56819032 | 83102 | 18: 56819579 |
| 83103 | 18: 56820099 | 83104 | 18: 56820288 | 83105 | 18: 56820485 | 83106 | 18: 56821287 | 83129 | 18: 57480828 |
| 83131 | 18: 57489304 | 83132 | 18: 57496016 | 83135 | 18: 57545249 | 83136 | 18: 57549761 | 83137 | 18: 57559274 |
| 83154 | 18: 57826436 | 83155 | 18: 57826437 | 83164 | 18: 57929781 | 83230 | 18: 58590500 | 83236 | 18: 58874702 |
| 83262 | 18: 60020922 | 83277 | 18: 60611535 | 83278 | 18: 60661061 | 83292 | 18: 61082028 | 83295 | 18: 61224849 |
| 83317 | 18: 61691526 | 83377 | 18: 62817960 | 83416 | 18: 63491911 | 83425 | 18: 63743739 | 83431 | 18: 63828080 |
| 83432 | 18: 63835095 | 83437 | 18: 63865767 | 83560 | 18: 65350149 | 83571 | 18: 65487247 | 83589 | 19: 153194 |
| 83605 | 19: 905203 | 83670 | 19: 3029129 | 83693 | 19: 3751571 | 83731 | 19: 4866448 | 83744 | 19: 5165734 |
| 83761 | 19: 5576963 | 83775 | 19: 5960251 | 83792 | 19: 6497947 | 83798 | 19: 6642039 | 83815 | 19: 6924319 |
| 83883 | 19: 8123606 | 83898 | 19: 8310681 | 83901 | 19: 8332089 | 83902 | 19: 8333798 | 83903 | 19: 8344332 |
| 83904 | 19: 8344354 | 83905 | 19: 8356008 | 83906 | 19: 8366630 | 83907 | 19: 8366749 | 83916 | 19: 8476307 |
| 83918 | 19: 8521460 | 83930 | 19: 8822288 | 83958 | 19: 9228505 | 83982 | 19: 9886007 | 83992 | 19: 10035802 |
| 83998 | 19: 10157460 | 83999 | 19: 10157552 | 84100 | 19: 12546324 | 84101 | 19: 12551345 | 84104 | 19: 12587276 |
| 84139 | 19: 13390721 | 84143 | 19: 13523234 | 84144 | 19: 13523312 | 84145 | 19: 13523394 | 84146 | 19: 13523599 |
| 84147 | 19: 13523669 | 84148 | 19: 13523920 | 84149 | 19: 13524217 | 84150 | 19: 13524534 | 84151 | 19: 13524690 |
| 84152 | 19: 13525738 | 84191 | 19: 14079144 | 84213 | 19: 14542746 | 84214 | 19: 14547882 | 84228 | 19: 14975147 |
| 84229 | 19: 14976930 | 84230 | 19: 14977190 | 84240 | 19: 15153935 | 84243 | 19: 15176665 | 84277 | 19: 15979571 |
| 84333 | 19: 16959557 | 84403 | 19: 18344112 | 84406 | 19: 18364792 | 84491 | 19: 20068707 | 84575 | 19: 21354124 |
| 84635 | 19: 22407739 | 84683 | 19: 23510771 | 84701 | 19: 23901688 | 84718 | 19: 24121007 | 84738 | 19: 24347182 |
| 84800 | 19: 25069906 | 84803 | 19: 25117527 | 84804 | 19: 25118864 | 84812 | 19: 25238500 | 84840 | 19: 25573317 |
| 84841 | 19: 25583861 | 84842 | 19: 25635561 | 84880 | 19: 26078995 | 84883 | 19: 26146723 | 84884 | 19: 26158447 |
| 84885 | 19: 26162940 | 84886 | 19: 26166792 | 84887 | 19: 26177724 | 84888 | 19: 26180605 | 84899 | 19: 26370592 |
| 84901 | 19: 26384973 | 84902 | 19: 26386173 | 84904 | 19: 26386998 | 84905 | 19: 26408649 | 84906 | 19: 26414194 |
| 84907 | 19: 26425088 | 84908 | 19: 26428880 | 84910 | 19: 26432937 | 84912 | 19: 26434534 | 84916 | 19: 26440887 |
| 84917 | 19: 26442377 | 84918 | 19: 26442417 | 84922 | 19: 26458156 | 84925 | 19: 26462109 | 84928 | 19: 26466738 |
| 84930 | 19: 26468564 | 84931 | 19: 26485885 | 84932 | 19: 26489724 | 84936 | 19: 26508139 | 84939 | 19: 26510614 |
| 84942 | 19: 26555053 | 84944 | 19: 26575215 | 84945 | 19: 26588232 | 84946 | 19: 26595700 | 84947 | 19: 26595755 |
| 84948 | 19: 26599010 | 84950 | 19: 26628925 | 84951 | 19: 26662321 | 84953 | 19: 26675470 | 84954 | 19: 26676204 |
| 84956 | 19: 26697210 | 84958 | 19: 26705636 | 84959 | 19: 26707790 | 84960 | 19: 26719756 | 84961 | 19: 26732472 |
| 84963 | 19: 26744245 | 85040 | 19: 28793496 | 85083 | 19: 29602130 | 85084 | 19: 29603381 | 85102 | 19: 29967023 |
| 85123 | 19: 30488002 | 85130 | 19: 30639326 | 85131 | 19: 30644348 | 85132 | 19: 30645410 | 85133 | 19: 30648555 |
| 85134 | 19: 30696412 | 85137 | 19: 30818025 | 85139 | 19: 30826714 | 85167 | 19: 31463142 | 85168 | 19: 31485672 |
| 85183 | 19: 31907703 | 85187 | 19: 31968664 | 85207 | 19: 32399697 | 85219 | 19: 32665116 | 85236 | 19: 33046409 |
| 85285 | 19: 34025535 | 85395 | 19: 35866723 | 85400 | 19: 35970366 | 85434 | 19: 36329939 | 85487 | 19: 37333280 |
| 85581 | 19: 40048911 | 85595 | 19: 40251758 | 85597 | 19: 40279227 | 85615 | 19: 40640928 | 85635 | 19: 40995967 |
| 85654 | 19: 41486757 | 85668 | 19: 41704492 | 85669 | 19: 41719174 | 85686 | 19: 41943823 | 85702 | 19: 42161619 |
| 85726 | 19: 42580737 | 85734 | 19: 42764834 | 85760 | 19: 43424335 | 85761 | 19: 43424535 | 85762 | 19: 43428799 |
| 85763 | 19: 43431509 | 85764 | 19: 43433116 | 85765 | 19: 43435751 | 85766 | 19: 43438458 | 85770 | 19: 43493558 |
| 85778 | 19: 43722064 | 85864 | 19: 45668089 | 85868 | 19: 45717594 | 85869 | 19: 45717924 | 85870 | 19: 45717976 |
| 85871 | 19: 45717979 | 85872 | 19: 45718107 | 85873 | 19: 45718307 | 85874 | 19: 45718340 | 85875 | 19: 45720202 |
| 85876 | 19: 45720264 | 85877 | 19: 45720488 | 85878 | 19: 45720862 | 85879 | 19: 45736048 | 85880 | 19: 45738664 |
| 85881 | 19: 45740485 | 85882 | 19: 45741144 | 85883 | 19: 45741389 | 85884 | 19: 45741952 | 85885 | 19: 45742152 |
| 85886 | 19: 45742153 | 85894 | 19: 45834449 | 85995 | 19: 48080018 | 86018 | 19: 48469710 | 86020 | 19: 48505288 |
| 86052 | 19: 49235212 | 86059 | 19: 49331336 | 86060 | 19: 49332593 | 86117 | 19: 50655487 | 86124 | 19: 50694652 |
| 86125 | 19: 50699155 | 86126 | 19: 50703436 | 86127 | 19: 50711021 | 86128 | 19: 50712302 | 86129 | 19: 50714204 |
| 86130 | 19: 50715174 | 86131 | 19: 50716477 | 86132 | 19: 50718075 | 86133 | 19: 50724757 | 86134 | 19: 50732339 |
| 86135 | 19: 50734637 | 86136 | 19: 50741020 | 86137 | 19: 50772016 | 86210 | 19: 51559279 | 86211 | 19: 51559359 |
| 86239 | 19: 52037026 | 86255 | 19: 52551695 | 86317 | 19: 53746270 | 86327 | 19: 53883618 | 86328 | 19: 53883934 |
| 86336 | 19: 54027650 | 86340 | 19: 54047174 | 86359 | 19: 54403366 | 86370 | 19: 54621153 | 86373 | 19: 54664915 |
| 86374 | 19: 54665643 | 86375 | 19: 54667304 | 86377 | 19: 54672612 | 86392 | 19: 54949422 | 86397 | 19: 55036335 |
| 86412 | 19: 55420287 | 86545 | 19: 56454067 | 86547 | 19: 56460077 | 86568 | 19: 56726108 | 86584 | 19: 57037210 |
| 86601 | 19: 57419304 | 86605 | 19: 57490381 | 86606 | 19: 57508171 | 86615 | 19: 57670291 | 86624 | 19: 57908956 |
| 86637 | 19: 58189519 | 86654 | 19: 58502690 | 86661 | 19: 58677753 | 86662 | 19: 58689620 | 86691 | 19: 59301702 |
| 86694 | 19: 59353477 | 86714 | 19: 59622681 | 86730 | 19: 59843660 | 86735 | 19: 59905716 | 86755 | 19: 60165330 |
| 86757 | 19: 60175972 | 86758 | 19: 60176459 | 86760 | 19: 60192077 | 86784 | 19: 60532635 | 86793 | 19: 60643681 |
| 86797 | 19: 60699603 | 86803 | 19: 60819634 | 86816 | 19: 61121915 | 86853 | 19: 61536597 | 86863 | 19: 61642528 |
| 86864 | 19: 61642559 | 86875 | 19: 61831896 | 86896 | 19: 62236608 | 86901 | 19: 62290414 | 86906 | 19: 62421264 |
| 86921 | 19: 62751964 | 86923 | 19: 62757779 | 86937 | 19: 62914703 | 86971 | 20: 465779 | 86984 | 20: 581428 |
| 86985 | 20: 583434 | 87026 | 20: 1587207 | 87034 | 20: 1637477 | 87046 | 20: 1840861 | 87049 | 20: 1872065 |
| 87081 | 20: 2302262 | 87098 | 20: 2623153 | 87109 | 20: 3049545 | 87110 | 20: 3050301 | 87111 | 20: 3052518 |
| 87112 | 20: 3056100 | 87113 | 20: 3056394 | 87114 | 20: 3061100 | 87115 | 20: 3061457 | 87116 | 20: 3062741 |
| 87117 | 20: 3064116 | 87118 | 20: 3064279 | 87119 | 20: 3065002 | 87120 | 20: 3065571 | 87121 | 20: 3065997 |
| 87185 | 20: 4419534 | 87443 | 20: 5918697 | 87517 | 20: 7068652 | 87549 | 20: 7706966 | 87560 | 20: 7984208 |
| 87598 | 20: 8931603 | 87671 | 20: 10580364 | 87672 | 20: 10581845 | 87697 | 20: 11193697 | 87724 | 20: 11666353 |
| 87731 | 20: 11849546 | 87737 | 20: 12016234 | 87780 | 20: 12743838 | 87791 | 20: 13014120 | 87797 | 20: 13145133 |
| 87811 | 20: 13530219 | 87817 | 20: 13767878 | 87836 | 20: 14332360 | 87852 | 20: 15273040 | 87864 | 20: 15564869 |
| 87875 | 20: 15869027 | 87906 | 20: 16621914 | 87909 | 20: 16650192 | 87961 | 20: 18107968 | 87991 | 20: 19026521 |
| 87993 | 20: 19083370 | 88011 | 20: 19606439 | 88019 | 20: 19755587 | 88078 | 20: 21474908 | 88083 | 20: 21584291 |
| 88117 | 20: 22063416 | 88191 | 20: 22598687 | 88200 | 20: 22757553 | 88209 | 20: 22931278 | 88259 | 20: 23864507 |
| 88277 | 20: 23991980 | 88323 | 20: 25147490 | 88359 | 20: 26491689 | 88360 | 20: 26496401 | 88361 | 20: 26502059 |
| 88362 | 20: 26504123 | 88363 | 20: 26514971 | 88364 | 20: 26518907 | 88366 | 20: 26522200 | 88406 | 20: 27651444 |
| 88407 | 20: 27665296 | 88420 | 20: 27980203 | 88434 | 20: 28277863 | 88467 | 20: 28789560 | 88482 | 20: 29236907 |
| 88526 | 20: 30411387 | 88535 | 20: 30681003 | 88568 | 20: 31716095 | 88572 | 20: 31786758 | 88574 | 20: 31871902 |
| 88685 | 20: 34576290 | 88806 | 20: 37374849 | 88817 | 20: 37489422 | 88837 | 20: 37861824 | 88857 | 20: 38404986 |
| 88885 | 20: 39228116 | 88889 | 20: 39268509 | 88899 | 20: 39457698 | 88902 | 20: 39467997 | 88907 | 20: 39535650 |
| 88913 | 20: 39565695 | 88931 | 20: 39982244 | 88942 | 20: 40219777 | 88959 | 20: 40746359 | 88976 | 20: 41050966 |
| 89002 | 20: 41628336 | 89045 | 20: 42462021 | 89117 | 20: 44485586 | 89140 | 20: 45144462 | 89147 | 20: 45457044 |
| 89148 | 20: 45547831 | 89150 | 20: 45567543 | 89154 | 20: 45642750 | 89156 | 20: 45675568 | 89158 | 20: 45694391 |
| 89174 | 20: 46029522 | 89175 | 20: 46040337 | 89176 | 20: 46040344 | 89177 | 20: 46043226 | 89189 | 20: 46349920 |
| 89197 | 20: 46707839 | 89206 | 20: 47136685 | 89214 | 20: 47190891 | 89228 | 20: 47736047 | 89277 | 20: 49126008 |
| 89278 | 20: 49182818 | 89301 | 20: 49941637 | 89316 | 20: 50311371 | 89348 | 20: 51482831 | 89384 | 20: 52684364 |
| 89407 | 20: 53375334 | 89412 | 20: 53445710 | 89417 | 20: 53542795 | 89468 | 20: 55149259 | 89470 | 20: 55168267 |
| 89471 | 20: 55169154 | 89474 | 20: 55188763 | 89476 | 20: 55206284 | 89477 | 20: 55207791 | 89481 | 20: 55321910 |
| 89488 | 20: 55507243 | 89626 | 20: 58393406 | 89647 | 20: 58770472 | 89651 | 20: 58809259 | 89694 | 20: 59879577 |
| 89697 | 20: 59895328 | 89741 | 20: 61269332 | 89769 | 20: 61983565 | 89777 | 20: 62202768 | 89791 | 20: 62523219 |
| 89834 | 20: 63451088 | 89878 | 20: 64194501 | 89901 | 20: 64754306 | 89915 | 20: 65165202 | 89925 | 20: 65480230 |
| 89931 | 20: 65608403 | 89992 | 20: 66984199 | 90045 | 20: 68049361 | 90056 | 20: 68247302 | 90060 | 20: 68312623 |
| 90065 | 20: 68359426 | 90126 | 20: 69498583 | 90156 | 20: 70136618 | 90175 | 20: 70711203 | 90290 | 21: 560149 |
| 90307 | 21: 1776376 | 90351 | 21: 3330534 | 90366 | 21: 3866539 | 90394 | 21: 4290924 | 90412 | 21: 4894437 |
| 90448 | 21: 5848777 | 90485 | 21: 7043405 | 90502 | 21: 7479548 | 90518 | 21: 7765157 | 90533 | 21: 8066355 |
| 90536 | 21: 8077420 | 90537 | 21: 8084411 | 90539 | 21: 8091687 | 90540 | 21: 8093756 | 90541 | 21: 8096108 |
| 90544 | 21: 8098084 | 90545 | 21: 8100095 | 90547 | 21: 8121506 | 90548 | 21: 8123911 | 90550 | 21: 8132548 |
| 90564 | 21: 8374844 | 90580 | 21: 8819472 | 90593 | 21: 9141650 | 90611 | 21: 9612990 | 90614 | 21: 9635812 |
| 90615 | 21: 9642385 | 90658 | 21: 10706242 | 90672 | 21: 10964762 | 90688 | 21: 11397271 | 90715 | 21: 12027109 |
| 90738 | 21: 12572473 | 90764 | 21: 13059367 | 90801 | 21: 13974032 | 90810 | 21: 14159859 | 90836 | 21: 14800811 |
| 90842 | 21: 14976378 | 90881 | 21: 15763479 | 90898 | 21: 16325852 | 90899 | 21: 16327666 | 90900 | 21: 16352286 |
| 90902 | 21: 16380714 | 90904 | 21: 16401319 | 90907 | 21: 16447288 | 90937 | 21: 16900535 | 90938 | 21: 16964578 |
| 90960 | 21: 17491219 | 90980 | 21: 18041395 | 91095 | 21: 19926364 | 91108 | 21: 20273386 | 91145 | 21: 20915737 |
| 91166 | 21: 21373080 | 91192 | 21: 21922090 | 91199 | 21: 22093113 | 91212 | 21: 22196208 | 91297 | 21: 24454479 |
| 91302 | 21: 24572190 | 91312 | 21: 24911518 | 91403 | 21: 27617328 | 91405 | 21: 27709157 | 91450 | 21: 29090583 |
| 91467 | 21: 29574040 | 91468 | 21: 29585741 | 91469 | 21: 29663335 | 91474 | 21: 29793976 | 91529 | 21: 31234804 |
| 91619 | 21: 33320463 | 91621 | 21: 33351272 | 91625 | 21: 33376637 | 91660 | 21: 33757910 | 91681 | 21: 34100944 |
| 91710 | 21: 34503640 | 91721 | 21: 34672141 | 91744 | 21: 35184835 | 91759 | 21: 35593323 | 91773 | 21: 35773499 |
| 91788 | 21: 36015961 | 91795 | 21: 36164785 | 91824 | 21: 36934454 | 91828 | 21: 37113932 | 91829 | 21: 37118207 |
| 91830 | 21: 37118300 | 91917 | 21: 39931787 | 91933 | 21: 40317765 | 91942 | 21: 40547674 | 91996 | 21: 41966823 |
| 92040 | 21: 42868063 | 92065 | 21: 43541273 | 92066 | 21: 43549420 | 92077 | 21: 43767846 | 92081 | 21: 43869821 |
| 92105 | 21: 44394987 | 92129 | 21: 44779372 | 92150 | 21: 45470174 | 92187 | 21: 46513102 | 92213 | 21: 47280539 |
| 92214 | 21: 47282065 | 92215 | 21: 47287202 | 92216 | 21: 47288654 | 92217 | 21: 47288689 | 92218 | 21: 47289181 |
| 92220 | 21: 47294064 | 92222 | 21: 47296827 | 92223 | 21: 47297692 | 92224 | 21: 47297903 | 92225 | 21: 47298236 |
| 92226 | 21: 47298622 | 92227 | 21: 47298661 | 92228 | 21: 47298894 | 92229 | 21: 47298942 | 92230 | 21: 47299468 |
| 92231 | 21: 47299536 | 92232 | 21: 47299742 | 92233 | 21: 47299950 | 92234 | 21: 47300166 | 92236 | 21: 47301069 |
| 92237 | 21: 47301092 | 92238 | 21: 47301705 | 92239 | 21: 47301914 | 92240 | 21: 47301979 | 92241 | 21: 47302160 |
| 92242 | 21: 47302405 | 92243 | 21: 47302771 | 92244 | 21: 47302793 | 92245 | 21: 47303150 | 92246 | 21: 47303326 |
| 92247 | 21: 47303615 | 92248 | 21: 47304710 | 92249 | 21: 47308349 | 92250 | 21: 47309250 | 92251 | 21: 47314809 |
| 92253 | 21: 47316827 | 92263 | 21: 47572280 | 92286 | 21: 48277772 | 92318 | 21: 49185947 | 92319 | 21: 49189566 |
| 92320 | 21: 49193705 | 92321 | 21: 49194179 | 92323 | 21: 49195976 | 92324 | 21: 49209645 | 92326 | 21: 49218212 |
| 92327 | 21: 49229215 | 92328 | 21: 49231400 | 92360 | 21: 50042557 | 92372 | 21: 50467495 | 92407 | 21: 51606765 |
| 92424 | 21: 52027284 | 92433 | 21: 52289877 | 92446 | 21: 52553332 | 92471 | 21: 53340020 | 92497 | 21: 54060629 |
| 92505 | 21: 54338413 | 92522 | 21: 54761061 | 92523 | 21: 54761327 | 92524 | 21: 54766467 | 92526 | 21: 54773661 |
| 92527 | 21: 54779429 | 92528 | 21: 54795770 | 92570 | 21: 55568962 | 92571 | 21: 55571357 | 92575 | 21: 55603036 |
| 92577 | 21: 55606404 | 92645 | 21: 56889237 | 92646 | 21: 56907066 | 92724 | 21: 57243795 | 92757 | 21: 58266418 |
| 92773 | 21: 58557250 | 92819 | 21: 59483269 | 92840 | 21: 59828548 | 92841 | 21: 59838076 | 92844 | 21: 59872593 |
| 92852 | 21: 59962135 | 92853 | 21: 59965050 | 92861 | 21: 60071151 | 92876 | 21: 60290443 | 92897 | 21: 60717146 |
| 92945 | 21: 61871337 | 92978 | 21: 62509823 | 92981 | 21: 62565907 | 92997 | 21: 62953263 | 93021 | 21: 63719399 |
| 93022 | 21: 63775744 | 93035 | 21: 64020970 | 93043 | 21: 64322263 | 93045 | 21: 64323466 | 93054 | 21: 64458851 |
| 93055 | 21: 64468813 | 93061 | 21: 64551281 | 93067 | 21: 64674944 | 93071 | 21: 64702159 | 93075 | 21: 64763778 |
| 93111 | 21: 65219263 | 93113 | 21: 65222945 | 93120 | 21: 65350726 | 93122 | 21: 65389454 | 93124 | 21: 65436920 |
| 93127 | 21: 65495121 | 93138 | 21: 65745850 | 93203 | 21: 67086290 | 93205 | 21: 67097791 | 93207 | 21: 67137594 |
| 93216 | 21: 67459394 | 93217 | 21: 67461089 | 93236 | 21: 67933900 | 93237 | 21: 67933901 | 93267 | 21: 68505684 |
| 93284 | 21: 68792488 | 93291 | 21: 68917772 | 93302 | 21: 69177495 | 93307 | 21: 69279748 | 93384 | 22: 1336082 |
| 93421 | 22: 2092658 | 93448 | 22: 2744263 | 93451 | 22: 2759833 | 93452 | 22: 2768731 | 93476 | 22: 3271155 |
| 93490 | 22: 3549590 | 93492 | 22: 3565222 | 93512 | 22: 4182249 | 93517 | 22: 4255887 | 93535 | 22: 4575207 |
| 93596 | 22: 5655551 | 93689 | 22: 7518330 | 93690 | 22: 7526334 | 93700 | 22: 7714187 | 93721 | 22: 8392537 |
| 93725 | 22: 8430070 | 93741 | 22: 9021813 | 93749 | 22: 9259333 | 93782 | 22: 9998563 | 93798 | 22: 10114240 |
| 93824 | 22: 10733620 | 93845 | 22: 11198997 | 93857 | 22: 11572590 | 93875 | 22: 11958308 | 93895 | 22: 12274925 |
| 93927 | 22: 12850291 | 93957 | 22: 13771925 | 93999 | 22: 14949092 | 94000 | 22: 14961326 | 94003 | 22: 15026827 |
| 94021 | 22: 15501897 | 94034 | 22: 15667257 | 94037 | 22: 15711517 | 94056 | 22: 16388677 | 94061 | 22: 16514054 |
| 94079 | 22: 16739297 | 94121 | 22: 17893434 | 94128 | 22: 18003077 | 94129 | 22: 18033855 | 94139 | 22: 18259710 |
| 94140 | 22: 18265709 | 94154 | 22: 18559489 | 94166 | 22: 18849789 | 94167 | 22: 18851178 | 94178 | 22: 19237876 |
| 94184 | 22: 19372836 | 94197 | 22: 19837982 | 94198 | 22: 19847979 | 94213 | 22: 20176939 | 94229 | 22: 20643619 |
| 94230 | 22: 20646235 | 94241 | 22: 20897046 | 94278 | 22: 21648235 | 94280 | 22: 21664858 | 94296 | 22: 21953904 |
| 94303 | 22: 22190483 | 94309 | 22: 22421295 | 94314 | 22: 22604015 | 94324 | 22: 22885054 | 94352 | 22: 23611781 |
| 94362 | 22: 23913434 | 94413 | 22: 25649715 | 94420 | 22: 25917253 | 94440 | 22: 26167092 | 94456 | 22: 26660485 |
| 94489 | 22: 27621315 | 94505 | 22: 28117808 | 94510 | 22: 28166101 | 94521 | 22: 28401484 | 94530 | 22: 28491057 |
| 94568 | 22: 29357579 | 94579 | 22: 29669451 | 94592 | 22: 29900170 | 94598 | 22: 30126912 | 94643 | 22: 31208133 |
| 94672 | 22: 32149285 | 94703 | 22: 33059262 | 94715 | 22: 33350762 | 94739 | 22: 34116071 | 94741 | 22: 34136171 |
| 94756 | 22: 34692656 | 94757 | 22: 34695203 | 94773 | 22: 35027110 | 94777 | 22: 35094672 | 94779 | 22: 35111999 |
| 94789 | 22: 35301748 | 94814 | 22: 35855583 | 94827 | 22: 36060507 | 94829 | 22: 36134170 | 94835 | 22: 36318773 |
| 94861 | 22: 37054760 | 94868 | 22: 37235828 | 94896 | 22: 37669398 | 94913 | 22: 38264582 | 94914 | 22: 38283974 |
| 94923 | 22: 38466243 | 94937 | 22: 38695645 | 94941 | 22: 38735989 | 94942 | 22: 38740955 | 94960 | 22: 39188813 |
| 94989 | 22: 39567152 | 94995 | 22: 39656245 | 94997 | 22: 39682658 | 95055 | 22: 41016011 | 95066 | 22: 41417903 |
| 95081 | 22: 41777356 | 95084 | 22: 41792218 | 95085 | 22: 41797029 | 95089 | 22: 41877932 | 95149 | 22: 43286201 |
| 95155 | 22: 43386150 | 95178 | 22: 44035160 | 95210 | 22: 44640940 | 95237 | 22: 45552005 | 95247 | 22: 45691255 |
| 95263 | 22: 45913562 | 95274 | 22: 46107748 | 95276 | 22: 46144763 | 95297 | 22: 46746909 | 95317 | 22: 47040045 |
| 95323 | 22: 47111303 | 95426 | 22: 49820256 | 95433 | 22: 49940593 | 95434 | 22: 49970190 | 95440 | 22: 50067364 |
| 95499 | 22: 51502260 | 95500 | 22: 51503045 | 95531 | 22: 52333167 | 95540 | 22: 52556549 | 95558 | 22: 52884674 |
| 95565 | 22: 52961463 | 95592 | 22: 53497101 | 95621 | 22: 54011562 | 95672 | 22: 55011602 | 95677 | 22: 55091224 |
| 95695 | 22: 55323748 | 95712 | 22: 55563765 | 95758 | 22: 56293581 | 95793 | 22: 56540115 | 95794 | 22: 56540668 |
| 95795 | 22: 56542873 | 95796 | 22: 56544054 | 95798 | 22: 56573939 | 95850 | 22: 57521486 | 95882 | 22: 57942911 |
| 95894 | 22: 58136676 | 95910 | 22: 58494061 | 95920 | 22: 58678398 | 95922 | 22: 58700947 | 95924 | 22: 58773917 |
| 95941 | 22: 59128229 | 95967 | 22: 59629485 | 95973 | 22: 59712330 | 95985 | 22: 59846886 | 96044 | 23: 774985 |
| 96050 | 23: 968798 | 96057 | 23: 1194189 | 96063 | 23: 1325388 | 96069 | 23: 1434392 | 96095 | 23: 2175010 |
| 96116 | 23: 2637687 | 96164 | 23: 3563217 | 96210 | 23: 4375629 | 96230 | 23: 4759481 | 96241 | 23: 4872845 |
| 96246 | 23: 5028069 | 96328 | 23: 6757709 | 96342 | 23: 7060820 | 96378 | 23: 7879887 | 96380 | 23: 7903756 |
| 96398 | 23: 8328018 | 96449 | 23: 9180909 | 96470 | 23: 9852522 | 96499 | 23: 10263390 | 96518 | 23: 10703356 |
| 96522 | 23: 10786602 | 96547 | 23: 11354447 | 96568 | 23: 11793160 | 96578 | 23: 12035181 | 96579 | 23: 12038531 |
| 96580 | 23: 12039512 | 96582 | 23: 12047337 | 96621 | 23: 12878298 | 96665 | 23: 13769527 | 96690 | 23: 14024594 |
| 96691 | 23: 14041498 | 96693 | 23: 14067842 | 96705 | 23: 14437275 | 96707 | 23: 14454128 | 96722 | 23: 14695783 |
| 96724 | 23: 14770597 | 96737 | 23: 14938161 | 96742 | 23: 15032946 | 96784 | 23: 15623072 | 96791 | 23: 15668311 |
| 96800 | 23: 15846192 | 96807 | 23: 16055984 | 96852 | 23: 17393860 | 96860 | 23: 17578001 | 96864 | 23: 17628981 |
| 96866 | 23: 17657910 | 96905 | 23: 18560341 | 96911 | 23: 18766462 | 96916 | 23: 18918643 | 96964 | 23: 19690687 |
| 96967 | 23: 19754433 | 96969 | 23: 19762716 | 96978 | 23: 19935139 | 96980 | 23: 19938929 | 96995 | 23: 20172115 |
| 97011 | 23: 20449980 | 97015 | 23: 20553981 | 97016 | 23: 20554380 | 97017 | 23: 20555345 | 97018 | 23: 20555464 |
| 97019 | 23: 20555512 | 97020 | 23: 20555877 | 97046 | 23: 21266809 | 97053 | 23: 21371538 | 97074 | 23: 22025892 |
| 97082 | 23: 22189816 | 97084 | 23: 22253261 | 97099 | 23: 22609075 | 97100 | 23: 22632358 | 97120 | 23: 23494563 |
| 97121 | 23: 23495169 | 97122 | 23: 23515623 | 97128 | 23: 23599173 | 97131 | 23: 23663060 | 97133 | 23: 23729292 |
| 97144 | 23: 23841412 | 97166 | 23: 24310745 | 97182 | 23: 24584592 | 97220 | 23: 24825942 | 97235 | 23: 24996122 |
| 97236 | 23: 25010458 | 97276 | 23: 25343657 | 97294 | 23: 25495218 | 97307 | 23: 25727838 | 97371 | 23: 27168873 |
| 97396 | 23: 27404973 | 97400 | 23: 27427125 | 97416 | 23: 27534819 | 97418 | 23: 27545798 | 97509 | 23: 28274177 |
| 97515 | 23: 28328479 | 97516 | 23: 28329217 | 97524 | 23: 28449487 | 97546 | 23: 28783122 | 97592 | 23: 29113119 |
| 97631 | 23: 30057510 | 97657 | 23: 30485309 | 97658 | 23: 30487092 | 97701 | 23: 31528534 | 97733 | 23: 32121998 |
| 97741 | 23: 32302519 | 97742 | 23: 32303395 | 97743 | 23: 32303732 | 97744 | 23: 32304775 | 97745 | 23: 32304794 |
| 97746 | 23: 32305503 | 97748 | 23: 32348823 | 97818 | 23: 33768810 | 97821 | 23: 33852032 | 97853 | 23: 34945913 |
| 97854 | 23: 34946818 | 97855 | 23: 34946868 | 97856 | 23: 34948119 | 97858 | 23: 35009819 | 97867 | 23: 35427374 |
| 97893 | 23: 36297029 | 97899 | 23: 36392895 | 97941 | 23: 37537593 | 97944 | 23: 37550813 | 97949 | 23: 37653049 |
| 97985 | 23: 38817188 | 97986 | 23: 38818425 | 98075 | 23: 39639975 | 98079 | 23: 39681488 | 98094 | 23: 39977867 |
| 98134 | 23: 40742268 | 98190 | 23: 41726170 | 98215 | 23: 42235597 | 98220 | 23: 42301353 | 98255 | 23: 43110372 |
| 98280 | 23: 43675243 | 98300 | 23: 44151531 | 98315 | 23: 44315474 | 98337 | 23: 44789129 | 98433 | 23: 46714094 |
| 98477 | 23: 47504887 | 98478 | 23: 47505469 | 98507 | 23: 47898321 | 98510 | 23: 47934746 | 98513 | 23: 47974902 |
| 98545 | 23: 48734960 | 98548 | 23: 48756252 | 98551 | 23: 48811144 | 98563 | 23: 49008700 | 98572 | 23: 49166634 |
| 98603 | 23: 49594224 | 98626 | 23: 49959432 | 98629 | 23: 50008283 | 98643 | 23: 50262003 | 98652 | 23: 50392532 |
| 98685 | 23: 51016737 | 98702 | 23: 51334319 | 98749 | 23: 52192597 | 98751 | 23: 52225791 | 98752 | 23: 52234534 |
| 98753 | 23: 52235518 | 98768 | 24: 90983 | 98820 | 24: 1186935 | 98822 | 24: 1209847 | 98833 | 24: 1549261 |
| 98855 | 24: 1914090 | 98878 | 24: 2417031 | 98920 | 24: 3326601 | 98976 | 24: 4463662 | 98988 | 24: 4937656 |
| 98990 | 24: 4964946 | 98992 | 24: 4997353 | 99007 | 24: 5345133 | 99010 | 24: 5453007 | 99011 | 24: 5453574 |
| 99019 | 24: 5665620 | 99110 | 24: 8068578 | 99117 | 24: 8174902 | 99126 | 24: 8403392 | 99156 | 24: 9223598 |
| 99164 | 24: 9437183 | 99183 | 24: 9782804 | 99185 | 24: 9813237 | 99187 | 24: 9859970 | 99197 | 24: 10080656 |
| 99198 | 24: 10082192 | 99199 | 24: 10083376 | 99200 | 24: 10084233 | 99201 | 24: 10085368 | 99202 | 24: 10085413 |
| 99203 | 24: 10085426 | 99204 | 24: 10085505 | 99205 | 24: 10085865 | 99206 | 24: 10086080 | 99207 | 24: 10086129 |
| 99208 | 24: 10086191 | 99209 | 24: 10087002 | 99210 | 24: 10087384 | 99211 | 24: 10087677 | 99212 | 24: 10088340 |
| 99213 | 24: 10089300 | 99214 | 24: 10090958 | 99215 | 24: 10092604 | 99216 | 24: 10092904 | 99217 | 24: 10093339 |
| 99218 | 24: 10093371 | 99219 | 24: 10093377 | 99220 | 24: 10093477 | 99221 | 24: 10093552 | 99222 | 24: 10093626 |
| 99223 | 24: 10099234 | 99224 | 24: 10100942 | 99225 | 24: 10101971 | 99226 | 24: 10102022 | 99227 | 24: 10102315 |
| 99228 | 24: 10102992 | 99229 | 24: 10104790 | 99230 | 24: 10106712 | 99231 | 24: 10112256 | 99232 | 24: 10120280 |
| 99233 | 24: 10120318 | 99234 | 24: 10125493 | 99235 | 24: 10127888 | 99236 | 24: 10128409 | 99237 | 24: 10128428 |
| 99238 | 24: 10128467 | 99239 | 24: 10129710 | 99240 | 24: 10129815 | 99241 | 24: 10131022 | 99242 | 24: 10131061 |
| 99243 | 24: 10131076 | 99244 | 24: 10131213 | 99245 | 24: 10131371 | 99246 | 24: 10131470 | 99247 | 24: 10131562 |
| 99248 | 24: 10132898 | 99249 | 24: 10132907 | 99250 | 24: 10134379 | 99251 | 24: 10134487 | 99252 | 24: 10134521 |
| 99253 | 24: 10134705 | 99254 | 24: 10134719 | 99255 | 24: 10134821 | 99256 | 24: 10134859 | 99257 | 24: 10134952 |
| 99258 | 24: 10134953 | 99259 | 24: 10136095 | 99260 | 24: 10137902 | 99261 | 24: 10137983 | 99262 | 24: 10139078 |
| 99263 | 24: 10139760 | 99264 | 24: 10140006 | 99265 | 24: 10140938 | 99266 | 24: 10143195 | 99267 | 24: 10143925 |
| 99268 | 24: 10144011 | 99269 | 24: 10144116 | 99270 | 24: 10150408 | 99271 | 24: 10151986 | 99272 | 24: 10153446 |
| 99273 | 24: 10155422 | 99291 | 24: 10548737 | 99303 | 24: 10838109 | 99313 | 24: 11081707 | 99347 | 24: 11895230 |
| 99374 | 24: 12693269 | 99378 | 24: 12740073 | 99397 | 24: 13255685 | 99401 | 24: 13395143 | 99406 | 24: 13474854 |
| 99411 | 24: 13599253 | 99431 | 24: 14202286 | 99432 | 24: 14280523 | 99460 | 24: 14990004 | 99467 | 24: 15215610 |
| 99471 | 24: 15361519 | 99472 | 24: 15366916 | 99495 | 24: 16259312 | 99498 | 24: 16327453 | 99514 | 24: 16832720 |
| 99517 | 24: 16888113 | 99550 | 24: 17884468 | 99553 | 24: 17978572 | 99564 | 24: 18322902 | 99588 | 24: 18993346 |
| 99621 | 24: 20085016 | 99710 | 24: 21387050 | 99713 | 24: 21396719 | 99714 | 24: 21407863 | 99721 | 24: 21479759 |
| 99823 | 24: 24049399 | 99835 | 24: 24317905 | 99862 | 24: 24997696 | 99899 | 24: 25782726 | 99918 | 24: 26095475 |
| 99926 | 24: 26225994 | 99966 | 24: 27495986 | 10000 | 24: 28406831 | 10000 | 24: 28544002 | 10000 | 24: 28549242 |
| 10002 | 24: 28829549 | 10002 | 24: 29046077 | 10007 | 24: 30123139 | 10019 | 24: 32865289 | 10024 | 24: 34044145 |
| 10025 | 24: 34324032 | 10028 | 24: 35161423 | 10029 | 24: 35363476 | 10031 | 24: 35787989 | 10034 | 24: 36327209 |
| 10034 | 24: 36389875 | 10043 | 24: 39349798 | 10047 | 24: 40319050 | 10048 | 24: 40319355 | 10048 | 24: 40320188 |
| 10048 | 24: 40320243 | 10048 | 24: 40320244 | 10048 | 24: 40320423 | 10048 | 24: 40320519 | 10048 | 24: 40320531 |
| 10048 | 24: 40320604 | 10048 | 24: 40320642 | 10048 | 24: 40320662 | 10049 | 24: 40346626 | 10049 | 24: 40351905 |
| 10062 | 24: 43050450 | 10062 | 24: 43264177 | 10064 | 24: 43757869 | 10064 | 24: 44070040 | 10065 | 24: 44312012 |
| 10066 | 24: 44454841 | 10067 | 24: 44630854 | 10068 | 24: 44900221 | 10069 | 24: 45024408 | 10073 | 24: 45883707 |
| 10074 | 24: 45892470 | 10074 | 24: 45893302 | 10074 | 24: 45900861 | 10074 | 24: 45906465 | 10074 | 24: 45915865 |
| 10074 | 24: 45916571 | 10074 | 24: 45917829 | 10075 | 24: 45935654 | 10075 | 24: 45946887 | 10075 | 24: 45961034 |
| 10075 | 24: 45965027 | 10075 | 24: 45966438 | 10075 | 24: 45966683 | 10075 | 24: 46019888 | 10076 | 24: 46021234 |
| 10076 | 24: 46031614 | 10076 | 24: 46039025 | 10076 | 24: 46039978 | 10076 | 24: 46040921 | 10076 | 24: 46047288 |
| 10076 | 24: 46050396 | 10076 | 24: 46052145 | 10079 | 24: 46519527 | 10090 | 24: 49563537 | 10100 | 24: 51861629 |
| 10100 | 24: 51877952 | 10102 | 24: 52249991 | 10102 | 24: 52250875 | 10103 | 24: 52380242 | 10103 | 24: 52572272 |
| 10103 | 24: 52579310 | 10108 | 24: 53806202 | 10115 | 24: 55068839 | 10121 | 24: 56483606 | 10121 | 24: 56487277 |
| 10123 | 24: 56927299 | 10123 | 24: 56931903 | 10124 | 24: 56935898 | 10129 | 24: 58059868 | 10130 | 24: 58143848 |
| 10131 | 24: 58393476 | 10132 | 24: 58514430 | 10133 | 24: 58871566 | 10138 | 24: 59956065 | 10138 | 24: 59956162 |
| 10141 | 24: 60385381 | 10141 | 24: 60520182 | 10145 | 24: 61136358 | 10147 | 24: 61660672 | 10151 | 25: 326413 |
| 10155 | 25: 1326500 | 10157 | 25: 1480308 | 10158 | 25: 1751313 | 10162 | 25: 2262957 | 10162 | 25: 2264339 |
| 10162 | 25: 2264421 | 10163 | 25: 2264518 | 10163 | 25: 2264598 | 10163 | 25: 2264608 | 10163 | 25: 2264757 |
| 10163 | 25: 2264786 | 10163 | 25: 2265149 | 10163 | 25: 2266070 | 10163 | 25: 2266258 | 10163 | 25: 2266451 |
| 10163 | 25: 2266559 | 10164 | 25: 2266588 | 10164 | 25: 2266682 | 10164 | 25: 2266930 | 10164 | 25: 2267017 |
| 10164 | 25: 2267496 | 10164 | 25: 2267627 | 10164 | 25: 2267877 | 10164 | 25: 2267895 | 10164 | 25: 2267998 |
| 10164 | 25: 2267999 | 10165 | 25: 2268074 | 10165 | 25: 2268874 | 10165 | 25: 2269160 | 10165 | 25: 2269161 |
| 10165 | 25: 2269296 | 10165 | 25: 2269538 | 10165 | 25: 2269574 | 10165 | 25: 2324283 | 10168 | 25: 2565490 |
| 10175 | 25: 3236023 | 10175 | 25: 3277932 | 10177 | 25: 3531364 | 10177 | 25: 3532276 | 10179 | 25: 3846717 |
| 10181 | 25: 4251363 | 10181 | 25: 4273072 | 10182 | 25: 4417956 | 10186 | 25: 5178359 | 10186 | 25: 5356785 |
| 10187 | 25: 5358991 | 10187 | 25: 5370164 | 10187 | 25: 5487714 | 10188 | 25: 5507570 | 10188 | 25: 5518043 |
| 10188 | 25: 5518349 | 10188 | 25: 5518412 | 10188 | 25: 5519000 | 10188 | 25: 5519033 | 10188 | 25: 5519088 |
| 10188 | 25: 5519241 | 10188 | 25: 5519377 | 10189 | 25: 5520929 | 10189 | 25: 5520954 | 10189 | 25: 5523535 |
| 10189 | 25: 5524957 | 10189 | 25: 5527480 | 10189 | 25: 5527963 | 10189 | 25: 5563162 | 10189 | 25: 5586659 |
| 10189 | 25: 5591911 | 10190 | 25: 5595291 | 10191 | 25: 5986235 | 10192 | 25: 6069855 | 10194 | 25: 6361028 |
| 10197 | 25: 7238527 | 10197 | 25: 7238686 | 10197 | 25: 7239004 | 10203 | 25: 8519593 | 10203 | 25: 8697220 |
| 10203 | 25: 8699100 | 10204 | 25: 8709323 | 10204 | 25: 8764411 | 10204 | 25: 8773984 | 10205 | 25: 8963960 |
| 10206 | 25: 9320778 | 10206 | 25: 9426676 | 10209 | 25: 10079477 | 10210 | 25: 10133416 | 10213 | 25: 10823773 |
| 10213 | 25: 10973032 | 10215 | 25: 11195704 | 10215 | 25: 11212796 | 10219 | 25: 12044897 | 10222 | 25: 12552168 |
| 10223 | 25: 12921926 | 10223 | 25: 12926787 | 10224 | 25: 13336640 | 10225 | 25: 13422767 | 10226 | 25: 13560729 |
| 10226 | 25: 13702632 | 10227 | 25: 14050888 | 10230 | 25: 14538936 | 10231 | 25: 14817768 | 10231 | 25: 14858436 |
| 10232 | 25: 14961033 | 10233 | 25: 15333545 | 10236 | 25: 15932671 | 10241 | 25: 16848884 | 10243 | 25: 17233999 |
| 10243 | 25: 17237242 | 10245 | 25: 17765386 | 10258 | 25: 21144052 | 10258 | 25: 21184609 | 10261 | 25: 21797730 |
| 10262 | 25: 22007781 | 10263 | 25: 22296079 | 10263 | 25: 22329885 | 10265 | 25: 22669357 | 10266 | 25: 22938890 |
| 10268 | 25: 23411155 | 10268 | 25: 23449496 | 10268 | 25: 23538270 | 10270 | 25: 23977366 | 10270 | 25: 23979613 |
| 10271 | 25: 24161354 | 10272 | 25: 24271708 | 10274 | 25: 24641425 | 10274 | 25: 24667454 | 10281 | 25: 25961592 |
| 10281 | 25: 26009553 | 10283 | 25: 26491942 | 10285 | 25: 26753501 | 10287 | 25: 27390789 | 10292 | 25: 27781936 |
| 10299 | 25: 29636939 | 10300 | 25: 29838430 | 10300 | 25: 29838448 | 10301 | 25: 29839578 | 10301 | 25: 29844263 |
| 10302 | 25: 30146443 | 10306 | 25: 31190020 | 10307 | 25: 31229333 | 10307 | 25: 31297977 | 10309 | 25: 31607175 |
| 10313 | 25: 32422851 | 10313 | 25: 32423232 | 10314 | 25: 32564396 | 10317 | 25: 33134898 | 10318 | 25: 33336358 |
| 10320 | 25: 33763001 | 10321 | 25: 34018085 | 10323 | 25: 34358250 | 10326 | 25: 34863162 | 10327 | 25: 34993980 |
| 10331 | 25: 35712925 | 10333 | 25: 35998185 | 10334 | 25: 36199960 | 10338 | 25: 36479714 | 10345 | 25: 37601488 |
| 10354 | 25: 39461505 | 10355 | 25: 39500543 | 10356 | 25: 39824171 | 10356 | 25: 39824495 | 10358 | 25: 40196620 |
| 10361 | 25: 40615618 | 10367 | 25: 41358682 | 10373 | 26: 1198222 | 10373 | 26: 1198334 | 10373 | 26: 1252329 |
| 10375 | 26: 1854681 | 10376 | 26: 2064954 | 10377 | 26: 2329446 | 10377 | 26: 2473264 | 10378 | 26: 2489945 |
| 10378 | 26: 2571028 | 10381 | 26: 3140966 | 10383 | 26: 3713174 | 10384 | 26: 3717033 | 10389 | 26: 4954983 |
| 10389 | 26: 5073658 | 10390 | 26: 5545287 | 10395 | 26: 7011510 | 10395 | 26: 7022453 | 10398 | 26: 7885953 |
| 10399 | 26: 8061770 | 10399 | 26: 8088819 | 10399 | 26: 8089138 | 10401 | 26: 8426892 | 10401 | 26: 8486858 |
| 10402 | 26: 8674404 | 10404 | 26: 9248331 | 10409 | 26: 10405639 | 10410 | 26: 10640932 | 10413 | 26: 11551549 |
| 10418 | 26: 12736512 | 10418 | 26: 12824626 | 10418 | 26: 12837527 | 10419 | 26: 12927334 | 10420 | 26: 13161640 |
| 10420 | 26: 13230527 | 10421 | 26: 13230553 | 10421 | 26: 13230674 | 10421 | 26: 13230708 | 10421 | 26: 13230970 |
| 10422 | 26: 13354058 | 10424 | 26: 14098514 | 10428 | 26: 15245070 | 10433 | 26: 16547356 | 10433 | 26: 16723176 |
| 10437 | 26: 17376800 | 10439 | 26: 17759010 | 10442 | 26: 18446389 | 10444 | 26: 18949002 | 10447 | 26: 19421103 |
| 10448 | 26: 19560868 | 10451 | 26: 20617024 | 10451 | 26: 20829028 | 10454 | 26: 21273598 | 10454 | 26: 21380205 |
| 10460 | 26: 23188643 | 10463 | 26: 24162535 | 10465 | 26: 24594195 | 10470 | 26: 25362443 | 10474 | 26: 25786538 |
| 10476 | 26: 26139139 | 10478 | 26: 26718349 | 10478 | 26: 26723790 | 10479 | 26: 26900204 | 10479 | 26: 26928555 |
| 10480 | 26: 27118119 | 10480 | 26: 27128850 | 10480 | 26: 27130646 | 10483 | 26: 27876991 | 10484 | 26: 28054644 |
| 10484 | 26: 28086960 | 10488 | 26: 29151186 | 10489 | 26: 29673193 | 10490 | 26: 29804085 | 10496 | 26: 31379196 |
| 10503 | 26: 33062542 | 10509 | 26: 34211364 | 10509 | 26: 34259597 | 10510 | 26: 34593375 | 10513 | 26: 35102465 |
| 10514 | 26: 35442752 | 10516 | 26: 35826945 | 10518 | 26: 36410448 | 10519 | 26: 36629017 | 10524 | 26: 38084778 |
| 10525 | 26: 38264488 | 10525 | 26: 38330297 | 10533 | 26: 40288755 | 10541 | 26: 42332291 | 10543 | 26: 42682604 |
| 10545 | 26: 43279886 | 10549 | 26: 44209268 | 10554 | 26: 45319249 | 10556 | 26: 45612666 | 10557 | 26: 45867614 |
| 10560 | 26: 46499606 | 10562 | 26: 46841840 | 10566 | 26: 47841265 | 10567 | 26: 47878623 | 10567 | 26: 48135137 |
| 10568 | 26: 48274013 | 10571 | 26: 48791917 | 10572 | 26: 49108450 | 10575 | 26: 49581277 | 10577 | 26: 50021533 |
| 10577 | 26: 50046769 | 10577 | 26: 50172060 | 10578 | 26: 50483114 | 10581 | 26: 50882810 | 10581 | 26: 51112488 |
| 10583 | 26: 51406962 | 10583 | 26: 51407053 | 10583 | 26: 51487956 | 10583 | 26: 51520196 | 10591 | 27: 1670150 |
| 10595 | 27: 2495166 | 10601 | 27: 4056109 | 10601 | 27: 4058537 | 10602 | 27: 4060363 | 10602 | 27: 4060531 |
| 10602 | 27: 4060533 | 10608 | 27: 5431758 | 10616 | 27: 7970865 | 10620 | 27: 8914781 | 10621 | 27: 8978523 |
| 10626 | 27: 9933403 | 10627 | 27: 9948916 | 10632 | 27: 11708333 | 10633 | 27: 11961138 | 10634 | 27: 12169807 |
| 10636 | 27: 12847147 | 10638 | 27: 13313622 | 10639 | 27: 13498868 | 10639 | 27: 13539506 | 10640 | 27: 13604776 |
| 10640 | 27: 13623320 | 10640 | 27: 13722354 | 10641 | 27: 13774707 | 10641 | 27: 13894721 | 10642 | 27: 13894736 |
| 10642 | 27: 13895111 | 10642 | 27: 13895260 | 10642 | 27: 13895824 | 10642 | 27: 13895841 | 10642 | 27: 13896100 |
| 10642 | 27: 13896107 | 10642 | 27: 13896340 | 10642 | 27: 13896382 | 10642 | 27: 13896475 | 10643 | 27: 13896495 |
| 10643 | 27: 13896544 | 10643 | 27: 13896557 | 10643 | 27: 13896621 | 10643 | 27: 13896676 | 10643 | 27: 13897078 |
| 10643 | 27: 13897184 | 10643 | 27: 13897225 | 10643 | 27: 13898435 | 10643 | 27: 13898864 | 10644 | 27: 13899961 |
| 10644 | 27: 13900283 | 10644 | 27: 13900709 | 10644 | 27: 13900927 | 10644 | 27: 13900986 | 10644 | 27: 13902739 |
| 10644 | 27: 13903517 | 10644 | 27: 13903525 | 10644 | 27: 13903671 | 10644 | 27: 13904229 | 10645 | 27: 13904269 |
| 10647 | 27: 14467858 | 10648 | 27: 14760458 | 10648 | 27: 14801389 | 10649 | 27: 15058579 | 10651 | 27: 15382547 |
| 10654 | 27: 15918980 | 10654 | 27: 16000987 | 10655 | 27: 16074052 | 10658 | 27: 16407816 | 10658 | 27: 16464527 |
| 10662 | 27: 17348090 | 10663 | 27: 17555135 | 10669 | 27: 19049000 | 10669 | 27: 19103942 | 10670 | 27: 19175688 |
| 10670 | 27: 19178944 | 10671 | 27: 19583156 | 10677 | 27: 20734233 | 10681 | 27: 21907032 | 10685 | 27: 23015129 |
| 10685 | 27: 23015519 | 10686 | 27: 23162886 | 10691 | 27: 24155175 | 10694 | 27: 24823413 | 10695 | 27: 24889870 |
| 10695 | 27: 25111470 | 10698 | 27: 25597037 | 10698 | 27: 25632047 | 10701 | 27: 26458109 | 10702 | 27: 26491670 |
| 10705 | 27: 27075217 | 10705 | 27: 27089901 | 10708 | 27: 27645675 | 10710 | 27: 28160084 | 10711 | 27: 28613312 |
| 10712 | 27: 28845373 | 10713 | 27: 29137179 | 10714 | 27: 29174979 | 10717 | 27: 29722415 | 10719 | 27: 30336321 |
| 10719 | 27: 30352172 | 10721 | 27: 30751847 | 10721 | 27: 30799722 | 10725 | 27: 31405475 | 10726 | 27: 31691203 |
| 10727 | 27: 31936569 | 10727 | 27: 31937059 | 10727 | 27: 31937078 | 10728 | 27: 31938645 | 10728 | 27: 31939482 |
| 10728 | 27: 31940571 | 10733 | 27: 32941799 | 10733 | 27: 32957490 | 10734 | 27: 33063853 | 10738 | 27: 34082682 |
| 10742 | 27: 35043316 | 10746 | 27: 35480823 | 10746 | 27: 35485331 | 10746 | 27: 35488554 | 10746 | 27: 35489257 |
| 10746 | 27: 35506982 | 10747 | 27: 35556513 | 10747 | 27: 35570251 | 10747 | 27: 35709604 | 10748 | 27: 35789498 |
| 10748 | 27: 35863653 | 10749 | 27: 35889925 | 10750 | 27: 36291522 | 10751 | 27: 36452785 | 10752 | 27: 36735236 |
| 10755 | 27: 37288845 | 10755 | 27: 37353731 | 10756 | 27: 37379099 | 10760 | 27: 38144204 | 10763 | 27: 38793276 |
| 10763 | 27: 38844060 | 10766 | 27: 39447790 | 10768 | 27: 40218010 | 10778 | 27: 42642792 | 10779 | 27: 43019477 |
| 10780 | 27: 43086918 | 10780 | 27: 43182706 | 10781 | 27: 43500535 | 10782 | 27: 43689399 | 10785 | 27: 44157299 |
| 10786 | 27: 44229484 | 10786 | 27: 44268404 | 10790 | 27: 45312149 | 10794 | 28: 1157605 | 10796 | 28: 1342853 |
| 10799 | 28: 2055151 | 10800 | 28: 2304836 | 10802 | 28: 3006891 | 10802 | 28: 3047863 | 10803 | 28: 3081340 |
| 10803 | 28: 3085107 | 10807 | 28: 5126492 | 10808 | 28: 5287434 | 10809 | 28: 5566930 | 10812 | 28: 5966745 |
| 10819 | 28: 7882083 | 10825 | 28: 9096925 | 10827 | 28: 9589074 | 10835 | 28: 12204786 | 10835 | 28: 12216204 |
| 10836 | 28: 12230473 | 10836 | 28: 12253261 | 10836 | 28: 12259647 | 10836 | 28: 12260824 | 10836 | 28: 12274548 |
| 10836 | 28: 12276973 | 10836 | 28: 12280112 | 10836 | 28: 12281177 | 10837 | 28: 12285449 | 10837 | 28: 12340669 |
| 10838 | 28: 12499169 | 10839 | 28: 12767375 | 10842 | 28: 13530571 | 10842 | 28: 13632810 | 10843 | 28: 14061176 |
| 10845 | 28: 14464098 | 10846 | 28: 14523784 | 10853 | 28: 16147418 | 10858 | 28: 17255203 | 10858 | 28: 17266697 |
| 10859 | 28: 17297973 | 10859 | 28: 17348454 | 10859 | 28: 17452718 | 10860 | 28: 17453580 | 10860 | 28: 17454068 |
| 10860 | 28: 17454138 | 10860 | 28: 17454593 | 10860 | 28: 17454620 | 10860 | 28: 17536228 | 10861 | 28: 17642513 |
| 10861 | 28: 17917993 | 10867 | 28: 19218143 | 10870 | 28: 20070339 | 10872 | 28: 20595474 | 10877 | 28: 22093298 |
| 10882 | 28: 23296337 | 10884 | 28: 23859369 | 10885 | 28: 24072970 | 10886 | 28: 24222637 | 10893 | 28: 25714994 |
| 10898 | 28: 26523238 | 10901 | 28: 27080357 | 10903 | 28: 27474956 | 10904 | 28: 27902972 | 10904 | 28: 27904071 |
| 10907 | 28: 28055097 | 10908 | 28: 28119648 | 10911 | 28: 28723390 | 10912 | 28: 28968643 | 10914 | 28: 29407005 |
| 10919 | 28: 30842659 | 10920 | 28: 31044910 | 10922 | 28: 31459804 | 10924 | 28: 31905889 | 10925 | 28: 32096800 |
| 10927 | 28: 32495037 | 10928 | 28: 32698642 | 10929 | 28: 32777537 | 10929 | 28: 32777974 | 10929 | 28: 32780910 |
| 10929 | 28: 32792000 | 10929 | 28: 32793533 | 10929 | 28: 32795812 | 10929 | 28: 32796515 | 10929 | 28: 32801458 |
| 10930 | 28: 32803767 | 10930 | 28: 32808502 | 10936 | 28: 34084210 | 10941 | 28: 34925017 | 10941 | 28: 34933980 |
| 10943 | 28: 35473082 | 10945 | 28: 35713729 | 10946 | 28: 36047723 | 10946 | 28: 36049682 | 10946 | 28: 36059895 |
| 10949 | 28: 36735400 | 10952 | 28: 37458115 | 10952 | 28: 37571711 | 10954 | 28: 37918674 | 10955 | 28: 38086633 |
| 10956 | 28: 38273052 | 10956 | 28: 38338458 | 10956 | 28: 38411536 | 10958 | 28: 38715969 | 10959 | 28: 39018331 |
| 10962 | 28: 39556352 | 10965 | 28: 40413981 | 10973 | 28: 40820229 | 10973 | 28: 40945850 | 10977 | 28: 41644978 |
| 10984 | 28: 42940481 | 10986 | 28: 43165757 | 10996 | 28: 45127262 | 10996 | 28: 45146124 | 10997 | 28: 45229830 |
| 11003 | 29: 1136794 | 11004 | 29: 1245711 | 11004 | 29: 1296904 | 11007 | 29: 1876223 | 11009 | 29: 2287359 |
| 11009 | 29: 2305683 | 11009 | 29: 2369666 | 11010 | 29: 2449190 | 11010 | 29: 2457832 | 11010 | 29: 2458133 |
| 11010 | 29: 2466616 | 11011 | 29: 2475067 | 11011 | 29: 2485044 | 11011 | 29: 2491210 | 11012 | 29: 2791675 |
| 11012 | 29: 2792329 | 11013 | 29: 2922954 | 11014 | 29: 3034446 | 11014 | 29: 3054628 | 11015 | 29: 3415853 |
| 11016 | 29: 3672128 | 11018 | 29: 4149425 | 11021 | 29: 4771466 | 11021 | 29: 4838513 | 11021 | 29: 4985399 |
| 11026 | 29: 6292926 | 11027 | 29: 6759181 | 11030 | 29: 7614767 | 11031 | 29: 7747910 | 11032 | 29: 8183109 |
| 11033 | 29: 8211095 | 11034 | 29: 8657356 | 11034 | 29: 8692066 | 11041 | 29: 10237310 | 11042 | 29: 10361704 |
| 11043 | 29: 10770679 | 11045 | 29: 11203337 | 11049 | 29: 11981461 | 11053 | 29: 12780503 | 11054 | 29: 13287093 |
| 11056 | 29: 13704499 | 11056 | 29: 13711224 | 11058 | 29: 14222266 | 11060 | 29: 14863923 | 11061 | 29: 15023531 |
| 11061 | 29: 15136113 | 11063 | 29: 15491770 | 11063 | 29: 15518673 | 11063 | 29: 15577523 | 11063 | 29: 15628215 |
| 11063 | 29: 15685760 | 11064 | 29: 15764983 | 11065 | 29: 16007380 | 11065 | 29: 16061265 | 11074 | 29: 19039096 |
| 11076 | 29: 19634491 | 11078 | 29: 20300393 | 11079 | 29: 20361512 | 11079 | 29: 20457751 | 11080 | 29: 20716777 |
| 11081 | 29: 20812973 | 11081 | 29: 20825149 | 11081 | 29: 20965169 | 11082 | 29: 21317409 | 11082 | 29: 21320200 |
| 11082 | 29: 21330819 | 11088 | 29: 22764191 | 11089 | 29: 23102280 | 11092 | 29: 23595059 | 11097 | 29: 24932130 |
| 11097 | 29: 24968087 | 11106 | 29: 27477867 | 11107 | 29: 27723748 | 11109 | 29: 28108791 | 111110 | 29: 28257705 |
| 11118 | 29: 29705618 | 11124 | 29: 30967685 | 11125 | 29: 31273255 | 11126 | 29: 31338655 | 11136 | 29: 33741610 |
| 11136 | 29: 33751577 | 11137 | 29: 33898078 | 11139 | 29: 34412457 | 11140 | 29: 34469913 | 11140 | 29: 34497844 |
| 11143 | 29: 35142257 | 11143 | 29: 35191995 | 11147 | 29: 35837500 | 11147 | 29: 35923286 | 11148 | 29: 36087955 |
| 11148 | 29: 36106402 | 11150 | 29: 36396078 | 11153 | 29: 37001499 | 11153 | 29: 37010699 | 11155 | 29: 37383025 |
| 11156 | 29: 37754439 | 11157 | 29: 37797892 | 11159 | 29: 39272372 | 11160 | 29: 39663800 | 11164 | 29: 40480123 |
| 11167 | 29: 40880222 | 11168 | 29: 41078094 | 11169 | 29: 41274073 | 11170 | 29: 41379850 | 11170 | 29: 41422028 |
| 11170 | 29: 41439204 | 11170 | 29: 41511246 | 11170 | 29: 41535408 | 11175 | 29: 42380020 | 11180 | 29: 43370086 |
| 11185 | 29: 44022611 | 11185 | 29: 44090714 | 11185 | 29: 44103946 | 11185 | 29: 44111412 | 11188 | 29: 44504806 |
| 11193 | 29: 45854845 | 11193 | 29: 45910725 | 11197 | 29: 46622198 | 11200 | 29: 47401443 | 11202 | 29: 47889180 |
| 11203 | 29: 47969493 | 11206 | 29: 48682297 | 11207 | 29: 48937511 | 11207 | 29: 48937944 | 11207 | 29: 48939020 |
| 11207 | 29: 49019691 | 11207 | 29: 49027895 | 11208 | 29: 49027958 | 11208 | 29: 49030254 | 11208 | 29: 49031741 |
| 11208 | 29: 49031778 | 11208 | 29: 49032495 | 11208 | 29: 49032522 | 11208 | 29: 49034373 | 11208 | 29: 49034762 |
| 11208 | 29: 49036958 | 11208 | 29: 49042618 | 11209 | 29: 49047332 | 11209 | 29: 49047368 | 11209 | 29: 49100292 |
| 11209 | 29: 49200650 | 11209 | 29: 49201144 | 11214 | 29: 50487594 | 11215 | 29: 50487813 | 11215 | 29: 50490270 |
| 11215 | 29: 50531220; | | | | | | | | |

and
the sites of the second marker panel are shown in the following table:

| NO. | Position | NO. | Position | NO. | Position | NO. | Position | NO. | Position |
|---|---|---|---|---|---|---|---|---|---|
| 23 | 1: 1010056 | 74 | 1: 1706307 | 78 | 1: 1817641 | 79 | 1: 1821203 | 81 | 1: 1836176 |
| 84 | 1: 1886288 | 87 | 1: 1979359 | 88 | 1: 1997766 | 91 | 1: 2032673 | 109 | 1: 2304587 |
| 112 | 1: 2390062 | 117 | 1: 2749113 | 125 | 1: 3018314 | 138 | 1: 3210484 | 143 | 1: 3282090 |
| 153 | 1: 3479131 | 158 | 1: 3520298 | 174 | 1: 3713849 | 175 | 1: 3716490 | 177 | 1: 3718949 |
| 178 | 1: 3720622 | 179 | 1: 3723615 | 192 | 1: 4021054 | 194 | 1: 4058825 | 197 | 1: 4081869 |
| 200 | 1: 4127064 | 201 | 1: 4130636 | 221 | 1: 4542229 | 245 | 1: 5107763 | 252 | 1: 5359288 |
| 274 | 1: 5770588 | 312 | 1: 6612693 | 320 | 1: 6739866 | 328 | 1: 6816263 | 329 | 1: 6827691 |
| 331 | 1: 6856676 | 333 | 1: 6963461 | 335 | 1: 7027764 | 342 | 1: 7064962 | 349 | 1: 7173547 |
| 351 | 1: 7269335 | 356 | 1: 7378233 | 362 | 1: 7438240 | 363 | 1: 7441167 | 368 | 1: 7637339 |
| 391 | 1: 8312182 | 395 | 1: 8473407 | 406 | 1: 8738949 | 420 | 1: 9069741 | 422 | 1: 9092818 |
| 426 | 1: 9259731 | 431 | 1: 9403159 | 432 | 1: 9442145 | 433 | 1: 9442252 | 434 | 1: 9442256 |
| 435 | 1: 9442802 | 436 | 1: 9467212 | 437 | 1: 9467322 | 439 | 1: 9505564 | 441 | 1: 9525931 |
| 449 | 1: 9675561 | 450 | 1: 9679977 | 451 | 1: 9699218 | 456 | 1: 9843069 | 460 | 1: 9929401 |
| 468 | 1: 10048554 | 472 | 1: 10225421 | 473 | 1: 10225456 | 474 | 1: 10231200 | 476 | 1: 10264906 |
| 477 | 1: 10308984 | 479 | 1: 10360268 | 487 | 1: 10501157 | 490 | 1: 10523191 | 491 | 1: 10537240 |
| 494 | 1: 10563513 | 495 | 1: 10565732 | 498 | 1: 10605121 | 502 | 1: 10771543 | 503 | 1: 10778102 |
| 505 | 1: 10809430 | 506 | 1: 10823521 | 520 | 1: 11223644 | 531 | 1: 11472343 | 535 | 1: 11550314 |
| 544 | 1: 12010431 | 555 | 1: 12378491 | 556 | 1: 12378514 | 566 | 1: 12644235 | 572 | 1: 12803499 |
| 573 | 1: 12803538 | 574 | 1: 12803609 | 584 | 1: 13280975 | 585 | 1: 13288892 | 587 | 1: 13292022 |
| 588 | 1: 13297157 | 591 | 1: 13398597 | 618 | 1: 13725800 | 645 | 1: 14655035 | 648 | 1: 14720755 |
| 668 | 1: 15201042 | 669 | 1: 15201103 | 670 | 1: 15201137 | 695 | 1: 15982024 | 702 | 1: 16155554 |
| 703 | 1: 16155577 | 704 | 1: 16155627 | 705 | 1: 16155668 | 714 | 1: 16447334 | 723 | 1: 16654742 |
| 741 | 1: 17350471 | 748 | 1: 17481344 | 749 | 1: 17481363 | 753 | 1: 17580720 | 774 | 1: 18459894 |
| 799 | 1: 19402363 | 800 | 1: 19409456 | 804 | 1: 19493132 | 814 | 1: 19676162 | 819 | 1: 19929109 |
| 833 | 1: 20279571 | 837 | 1: 20407748 | 840 | 1: 20480157 | 845 | 1: 20570353 | 847 | 1: 20621543 |
| 857 | 1: 20823430 | 860 | 1: 20921794 | 861 | 1: 20974759 | 863 | 1: 21005976 | 868 | 1: 21167645 |
| 872 | 1: 21346196 | 883 | 1: 21646634 | 890 | 1: 21783397 | 896 | 1: 21921230 | 902 | 1: 22076454 |
| 925 | 1: 22688749 | 933 | 1: 22949333 | 947 | 1: 23444110 | 952 | 1: 23709011 | 953 | 1: 23709064 |
| 990 | 1: 24753266 | 993 | 1: 24782983 | 1010 | 1: 25234761 | 1011 | 1: 25234832 | 1023 | 1: 25497647 |
| 1056 | 1: 26533932 | 1058 | 1: 26552621 | 1061 | 1: 26559576 | 1063 | 1: 26611592 | 1067 | 1: 26755277 |
| 1070 | 1: 26880560 | 1072 | 1: 26882959 | 1077 | 1: 26969554 | 1082 | 1: 27132369 | 1084 | 1: 27171038 |
| 1092 | 1: 27401121 | 1134 | 1: 29068209 | 1144 | 1: 29340605 | 1147 | 1: 29403350 | 1151 | 1: 29458506 |
| 1158 | 1: 29712048 | 1165 | 1: 29884320 | 1170 | 1: 29949770 | 1180 | 1: 30194782 | 1201 | 1: 30897798 |
| 1219 | 1: 31546632 | 1227 | 1: 31736868 | 1249 | 1: 32659120 | 1262 | 1: 33136711 | 1265 | 1: 33292309 |
| 1270 | 1: 33496557 | 1272 | 1: 33533012 | 1289 | 1: 33884832 | 1300 | 1: 34174940 | 1309 | 1: 34602877 |
| 1311 | 1: 34663000 | 1313 | 1: 34664612 | 1317 | 1: 34824821 | 1318 | 1: 34824930 | 1321 | 1: 34924925 |
| 1324 | 1: 35000632 | 1331 | 1: 35082343 | 1344 | 1: 35345367 | 1349 | 1: 35407003 | 1362 | 1: 35733756 |
| 1370 | 1: 36018590 | 1372 | 1: 36111561 | 1379 | 1: 36303126 | 1389 | 1: 36436645 | 1392 | 1: 36459421 |
| 1393 | 1: 36459510 | 1402 | 1: 36697707 | 1408 | 1: 36828697 | 1414 | 1: 37032363 | 1415 | 1: 37032440 |
| 1426 | 1: 37411283 | 1428 | 1: 37442242 | 1434 | 1: 37638872 | 1438 | 1: 37686047 | 1440 | 1: 37725772 |
| 1450 | 1: 37956983 | 1453 | 1: 38013891 | 1468 | 1: 38540153 | 1469 | 1: 38599466 | 1476 | 1: 38720880 |
| 1494 | 1: 39249574 | 1505 | 1: 39697516 | 1534 | 1: 40506922 | 1538 | 1: 40629765 | 1548 | 1: 40789010 |
| 1573 | 1: 41686185 | 1579 | 1: 41784113 | 1580 | 1: 41788052 | 1581 | 1: 41795018 | 1582 | 1: 41795589 |
| 1583 | 1: 41796750 | 1584 | 1: 41797989 | 1586 | 1: 41805753 | 1587 | 1: 41835927 | 1588 | 1: 41836033 |
| 1590 | 1: 41886688 | 1600 | 1: 42107409 | 1601 | 1: 42131473 | 1604 | 1: 42159211 | 1605 | 1: 42170788 |
| 1609 | 1: 42224416 | 1611 | 1: 42265122 | 1612 | 1: 42285326 | 1613 | 1: 42293500 | 1619 | 1: 42519517 |
| 1633 | 1: 42917734 | 1640 | 1: 43141355 | 1643 | 1: 43211702 | 1645 | 1: 43225435 | 1647 | 1: 43292695 |
| 1652 | 1: 43443436 | 1656 | 1: 43496586 | 1668 | 1: 43756861 | 1670 | 1: 43772333 | 1672 | 1: 43787631 |
| 1684 | 1: 44030562 | 1686 | 1: 44124010 | 1688 | 1: 44137020 | 1690 | 1: 44236960 | 1700 | 1: 44393495 |
| 1701 | 1: 44422846 | 1708 | 1: 44623944 | 1712 | 1: 44766328 | 1714 | 1: 44820415 | 1715 | 1: 44820480 |
| 1716 | 1: 44820576 | 1717 | 1: 44847495 | 1732 | 1: 44947997 | 1733 | 1: 44956505 | 1734 | 1: 44959367 |
| 1735 | 1: 44960388 | 1737 | 1: 44995524 | 1738 | 1: 44998217 | 1740 | 1: 45030214 | 1741 | 1: 45036157 |
| 1744 | 1: 45061133 | 1748 | 1: 45196717 | 1750 | 1: 45216991 | 1759 | 1: 45365527 | 1760 | 1: 45372759 |
| 1784 | 1: 46185873 | 1793 | 1: 46488716 | 1821 | 1: 47439819 | 1822 | 1: 47457097 | 1823 | 1: 47465147 |
| 1829 | 1: 47631393 | 1852 | 1: 48538887 | 1853 | 1: 48538897 | 1866 | 1: 48823158 | 1893 | 1: 49844520 |
| 1907 | 1: 50327235 | 1913 | 1: 50531202 | 1916 | 1: 50627944 | 1917 | 1: 50631061 | 1918 | 1: 50631100 |
| 1919 | 1: 50637655 | 1924 | 1: 50846942 | 1925 | 1: 50856429 | 1926 | 1: 50874802 | 1928 | 1: 50934316 |
| 1931 | 1: 50978540 | 1934 | 1: 51048823 | 1945 | 1: 51501180 | 1958 | 1: 51956854 | 1960 | 1: 52001408 |
| 1974 | 1: 52418784 | 1975 | 1: 52420721 | 1977 | 1: 52468623 | 1979 | 1: 52499614 | 1980 | 1: 52499623 |
| 1982 | 1: 52592314 | 1983 | 1: 52616071 | 1984 | 1: 52639898 | 1989 | 1: 52845116 | 1999 | 1: 53149622 |
| 2008 | 1: 53293355 | 2011 | 1: 53338531 | 2015 | 1: 53534215 | 2034 | 1: 53685551 | 2036 | 1: 53685641 |
| 2037 | 1: 53685662 | 2038 | 1: 53685710 | 2043 | 1: 53760822 | 2049 | 1: 53972219 | 2055 | 1: 54092473 |
| 2056 | 1: 54092637 | 2057 | 1: 54092679 | 2058 | 1: 54093927 | 2059 | 1: 54101343 | 2068 | 1: 54343019 |
| 2076 | 1: 54460688 | 2088 | 1: 54757168 | 2089 | 1: 54766036 | 2096 | 1: 55026386 | 2097 | 1: 55026433 |
| 2108 | 1: 55421744 | 2110 | 1: 55482490 | 2121 | 1: 55927365 | 2122 | 1: 55964487 | 2123 | 1: 55970922 |
| 2132 | 1: 56253478 | 2138 | 1: 56370458 | 2142 | 1: 56472558 | 2145 | 1: 56498727 | 2147 | 1: 56540591 |
| 2154 | 1: 56759097 | 2162 | 1: 56937696 | 2165 | 1: 56975870 | 2170 | 1: 57137491 | 2173 | 1: 57217658 |
| 2174 | 1: 57217807 | 2175 | 1: 57251079 | 2176 | 1: 57266134 | 2178 | 1: 57309255 | 2181 | 1: 57398938 |
| 2184 | 1: 57490618 | 2185 | 1: 57560306 | 2197 | 1: 57870387 | 2198 | 1: 57917830 | 2203 | 1: 58030359 |
| 2205 | 1: 58075432 | 2206 | 1: 58075463 | 2207 | 1: 58075499 | 2208 | 1: 58075521 | 2209 | 1: 58078354 |
| 2210 | 1: 58078466 | 2211 | 1: 58097510 | 2221 | 1: 58305398 | 2222 | 1: 58305455 | 2223 | 1: 58315322 |
| 2224 | 1: 58315443 | 2225 | 1: 58315990 | 2231 | 1: 58540410 | 2232 | 1: 58618667 | 2236 | 1: 58750747 |
| 2237 | 1: 58773333 | 2240 | 1: 58818870 | 2250 | 1: 59034012 | 2252 | 1: 59104762 | 2258 | 1: 59200011 |
| 2259 | 1: 59203569 | 2263 | 1: 59371385 | 2270 | 1: 59656138 | 2279 | 1: 59830679 | 2293 | 1: 60123244 |
| 2318 | 1: 60784872 | 2325 | 1: 60918912 | 2330 | 1: 61097118 | 2333 | 1: 61128757 | 2336 | 1: 61160140 |
| 2369 | 1: 62066946 | 2370 | 1: 62092465 | 2386 | 1: 62457515 | 2399 | 1: 62621737 | 2402 | 1: 62637571 |
| 2406 | 1: 62817788 | 2407 | 1: 62817866 | 2428 | 1: 63139698 | 2438 | 1: 63427104 | 2440 | 1: 63476336 |
| 2441 | 1: 63517812 | 2451 | 1: 63851933 | 2456 | 1: 64008039 | 2461 | 1: 64183671 | 2463 | 1: 64200486 |
| 2464 | 1: 64200539 | 2466 | 1: 64262468 | 2477 | 1: 64431800 | 2478 | 1: 64462597 | 2479 | 1: 64462675 |
| 2480 | 1: 64463152 | 2481 | 1: 64463254 | 2482 | 1: 64467138 | 2483 | 1: 64467211 | 2486 | 1: 64505435 |
| 2496 | 1: 64824564 | 2502 | 1: 64940185 | 2509 | 1: 65157725 | 2512 | 1: 65199200 | 2513 | 1: 65216449 |
| 2514 | 1: 65224381 | 2515 | 1: 65228988 | 2516 | 1: 65239311 | 2520 | 1: 65377133 | 2545 | 1: 66100053 |
| 2547 | 1: 66136711 | 2557 | 1: 66502772 | 2558 | 1: 66511698 | 2559 | 1: 66515809 | 2561 | 1: 66547988 |
| 2569 | 1: 66694865 | 2581 | 1: 67173187 | 2588 | 1: 67320099 | 2590 | 1: 67331681 | 2591 | 1: 67411736 |
| 2593 | 1: 67432038 | 2594 | 1: 67439085 | 2596 | 1: 67473778 | 2604 | 1: 67760033 | 2608 | 1: 67861511 |
| 2618 | 1: 68238481 | 2619 | 1: 68259739 | 2623 | 1: 68332459 | 2627 | 1: 68449381 | 2628 | 1: 68488305 |
| 2630 | 1: 68501205 | 2631 | 1: 68517809 | 2635 | 1: 68649543 | 2637 | 1: 68680831 | 2638 | 1: 68682422 |
| 2639 | 1: 68682461 | 2640 | 1: 68690274 | 2644 | 1: 68784544 | 2646 | 1: 68792494 | 2647 | 1: 68793488 |
| 2648 | 1: 68793540 | 2649 | 1: 68793542 | 2650 | 1: 68793543 | 2651 | 1: 68797693 | 2656 | 1: 68869228 |
| 2663 | 1: 68996534 | 2668 | 1: 69017745 | 2670 | 1: 69042317 | 2671 | 1: 69047274 | 2672 | 1: 69047282 |
| 2673 | 1: 69047320 | 2679 | 1: 69124262 | 2682 | 1: 69203913 | 2684 | 1: 69217319 | 2685 | 1: 69217672 |
| 2698 | 1: 69582986 | 2699 | 1: 69585403 | 2704 | 1: 69764910 | 2712 | 1: 69883156 | 2717 | 1: 69982394 |
| 2720 | 1: 70106968 | 2725 | 1: 70231031 | 2726 | 1: 70231761 | 2727 | 1: 70248171 | 2730 | 1: 70343458 |
| 2732 | 1: 70365636 | 2734 | 1: 70390927 | 2741 | 1: 70466635 | 2742 | 1: 70466719 | 2743 | 1: 70466765 |
| 2744 | 1: 70468358 | 2745 | 1: 70473449 | 2746 | 1: 70473451 | 2747 | 1: 70473452 | 2748 | 1: 70473471 |
| 2749 | 1: 70473493 | 2750 | 1: 70485290 | 2751 | 1: 70485353 | 2753 | 1: 70494055 | 2754 | 1: 70497518 |
| 2755 | 1: 70500089 | 2762 | 1: 70616796 | 2771 | 1: 70783887 | 2772 | 1: 70791872 | 2773 | 1: 70801580 |
| 2790 | 1: 71415748 | 2791 | 1: 71416509 | 2792 | 1: 71416751 | 2793 | 1: 71416881 | 2794 | 1: 71416890 |
| 2803 | 1: 71609698 | 2817 | 1: 72069890 | 2819 | 1: 72081243 | 2820 | 1: 72090597 | 2834 | 1: 72454020 |
| 2836 | 1: 72484009 | 2839 | 1: 72519103 | 2849 | 1: 72679096 | 2850 | 1: 72679109 | 2851 | 1: 72679167 |
| 2854 | 1: 72714931 | 2855 | 1: 72726681 | 2856 | 1: 72732857 | 2859 | 1: 72803314 | 2863 | 1: 72952213 |
| 2864 | 1: 72955756 | 2866 | 1: 73093141 | 2871 | 1: 73187152 | 2872 | 1: 73207902 | 2879 | 1: 73477972 |
| 2883 | 1: 73591733 | 2887 | 1: 73663050 | 2888 | 1: 73671409 | 2890 | 1: 73744599 | 2898 | 1: 73902125 |
| 2902 | 1: 73954764 | 2908 | 1: 74183606 | 2915 | 1: 74460825 | 2917 | 1: 74460955 | 2918 | 1: 74489282 |
| 2919 | 1: 74490359 | 2922 | 1: 74553801 | 2935 | 1: 74976635 | 2957 | 1: 75592224 | 2960 | 1: 75682742 |
| 2967 | 1: 75798251 | 2968 | 1: 75798252 | 2969 | 1: 75798317 | 2972 | 1: 75852855 | 2980 | 1: 76051714 |
| 3013 | 1: 77045567 | 3020 | 1: 77255234 | 3029 | 1: 77397999 | 3032 | 1: 77443583 | 3033 | 1: 77469880 |
| 3036 | 1: 77593114 | 3038 | 1: 77616604 | 3040 | 1: 77652308 | 3044 | 1: 77698757 | 3048 | 1: 77727505 |
| 3054 | 1: 77958907 | 3074 | 1: 78375438 | 3077 | 1: 78445734 | 3082 | 1: 78556250 | 3083 | 1: 78566682 |
| 3093 | 1: 78911959 | 3095 | 1: 78989138 | 3098 | 1: 79082187 | 3101 | 1: 79138960 | 3102 | 1: 79169504 |
| 3105 | 1: 79213373 | 3107 | 1: 79242860 | 3110 | 1: 79298445 | 3114 | 1: 79357690 | 3118 | 1: 79485282 |
| 3123 | 1: 79562994 | 3156 | 1: 80023498 | 3161 | 1: 80069552 | 3163 | 1: 80074105 | 3172 | 1: 80391190 |
| 3173 | 1: 80413261 | 3183 | 1: 80685030 | 3192 | 1: 80940339 | 3196 | 1: 81028408 | 3201 | 1: 81051808 |
| 3208 | 1: 81170302 | 3209 | 1: 81183483 | 3215 | 1: 81348584 | 3229 | 1: 81706937 | 3235 | 1: 81878324 |
| 3240 | 1: 82041541 | 3241 | 1: 82041559 | 3242 | 1: 82047704 | 3246 | 1: 82149539 | 3258 | 1: 82473626 |
| 3264 | 1: 82519837 | 3265 | 1: 82541328 | 3266 | 1: 82541450 | 3268 | 1: 82558245 | 3273 | 1: 82643904 |
| 3276 | 1: 82705721 | 3278 | 1: 82758391 | 3284 | 1: 83070275 | 3285 | 1: 83097205 | 3286 | 1: 83133001 |
| 3291 | 1: 83278833 | 3292 | 1: 83284140 | 3293 | 1: 83330542 | 3306 | 1: 83810343 | 3310 | 1: 83974373 |
| 3312 | 1: 84000147 | 3315 | 1: 84047005 | 3316 | 1: 84058598 | 3338 | 1: 85042289 | 3344 | 1: 85087002 |
| 3346 | 1: 85130568 | 3368 | 1: 85947378 | 3369 | 1: 85948692 | 3371 | 1: 85976228 | 3374 | 1: 86122181 |
| 3375 | 1: 86146073 | 3376 | 1: 86156408 | 3377 | 1: 86160591 | 3378 | 1: 86177464 | 3379 | 1: 86185749 |
| 3380 | 1: 86186427 | 3394 | 1: 86631754 | 3406 | 1: 86967586 | 3420 | 1: 87399098 | 3421 | 1: 87399338 |
| 3422 | 1: 87414899 | 3428 | 1: 87574019 | 3431 | 1: 87648729 | 3434 | 1: 87752223 | 3441 | 1: 88012422 |
| 3467 | 1: 88872312 | 3468 | 1: 88874634 | 3469 | 1: 88880495 | 3471 | 1: 88896168 | 3472 | 1: 88908213 |
| 3474 | 1: 88987015 | 3476 | 1: 89086745 | 3478 | 1: 89097927 | 3479 | 1: 89098024 | 3482 | 1: 89224144 |
| 3484 | 1: 89300003 | 3486 | 1: 89343474 | 3487 | 1: 89349576 | 3488 | 1: 89383250 | 3492 | 1: 89498954 |
| 3494 | 1: 89539395 | 3495 | 1: 89539442 | 3508 | 1: 89796658 | 3509 | 1: 89799413 | 3522 | 1: 90043900 |
| 3524 | 1: 90075379 | 3550 | 1: 90588788 | 3552 | 1: 90615269 | 3554 | 1: 90648889 | 3576 | 1: 91255865 |
| 3577 | 1: 91285295 | 3593 | 1: 91706481 | 3596 | 1: 91737921 | 3597 | 1: 91739554 | 3602 | 1: 91928880 |
| 3609 | 1: 92184004 | 3623 | 1: 92503312 | 3668 | 1: 93883957 | 3670 | 1: 93930496 | 3674 | 1: 94035144 |
| 3675 | 1: 94035267 | 3680 | 1: 94131411 | 3681 | 1: 94134092 | 3699 | 1: 94576910 | 3709 | 1: 94877027 |
| 3729 | 1: 95423137 | 3730 | 1: 95439594 | 3731 | 1: 95455255 | 3733 | 1: 95478530 | 3734 | 1: 95494762 |
| 3745 | 1: 95806271 | 3747 | 1: 95893257 | 3752 | 1: 96002349 | 3754 | 1: 96026129 | 3756 | 1: 96084552 |
| 3757 | 1: 96085021 | 3760 | 1: 96180861 | 3764 | 1: 96218350 | 3765 | 1: 96220152 | 3770 | 1: 96344874 |
| 3779 | 1: 96474332 | 3784 | 1: 96640052 | 3801 | 1: 97152251 | 3819 | 1: 97537514 | 3832 | 1: 97878133 |
| 3843 | 1: 98148099 | 3847 | 1: 98313182 | 3857 | 1: 98673491 | 3867 | 1: 98891260 | 3876 | 1: 99200317 |
| 3882 | 1: 99297524 | 3883 | 1: 99323405 | 3886 | 1: 99418906 | 3890 | 1: 99501232 | 3906 | 1: 99941713 |
| 3914 | 1: 100198782 | 3917 | 1: 100296109 | 3919 | 1: 100321230 | 3927 | 1: 100567937 | 3948 | 1: 101273216 |
| 3969 | 1: 101894473 | 3980 | 1: 102388029 | 3981 | 1: 102388112 | 3988 | 1: 102582187 | 3990 | 1: 102591751 |
| 3993 | 1: 102638499 | 3995 | 1: 102646032 | 3997 | 1: 102698749 | 4004 | 1: 102911722 | 4008 | 1: 103023798 |
| 4037 | 1: 104209003 | 4038 | 1: 104263712 | 4045 | 1: 104522827 | 4053 | 1: 104831357 | 4058 | 1: 104915762 |
| 4060 | 1: 104987991 | 4061 | 1: 105015424 | 4074 | 1: 105367807 | 4079 | 1: 105447199 | 4103 | 1: 106048772 |
| 4108 | 1: 106226124 | 4111 | 1: 106284785 | 4123 | 1: 106497025 | 4144 | 1: 107326886 | 4149 | 1: 107424074 |
| 4153 | 1: 107500490 | 4154 | 1: 107538852 | 4155 | 1: 107544465 | 4156 | 1: 107544536 | 4161 | 1: 107781031 |
| 4164 | 1: 107863018 | 4170 | 1: 108114016 | 4189 | 1: 108515165 | 4191 | 1: 108556182 | 4192 | 1: 108556227 |
| 4203 | 1: 108851719 | 4204 | 1: 108851745 | 4205 | 1: 108853578 | 4206 | 1: 108854832 | 4207 | 1: 108870778 |
| 4208 | 1: 108874776 | 4209 | 1: 108881505 | 4210 | 1: 108884859 | 4211 | 1: 108894708 | 4212 | 1: 108894728 |
| 4213 | 1: 108896221 | 4230 | 1: 109527893 | 4231 | 1: 109527919 | 4245 | 1: 109820651 | 4250 | 1: 109870082 |
| 4266 | 1: 110126279 | 4267 | 1: 110175621 | 4269 | 1: 110196398 | 4273 | 1: 110293441 | 4276 | 1: 110341970 |
| 4277 | 1: 110342054 | 4278 | 1: 110342111 | 4283 | 1: 110476863 | 4288 | 1: 110573226 | 4289 | 1: 110590869 |
| 4292 | 1: 110633655 | 4293 | 1: 110653778 | 4295 | 1: 110665646 | 4296 | 1: 110666740 | 4302 | 1: 110816709 |
| 4309 | 1: 111186619 | 4310 | 1: 111194151 | 4311 | 1: 111199408 | 4312 | 1: 111247418 | 4313 | 1: 111268099 |
| 4325 | 1: 111708777 | 4327 | 1: 111748641 | 4331 | 1: 111775768 | 4332 | 1: 111775805 | 4333 | 1: 111798208 |
| 4345 | 1: 112208772 | 4353 | 1: 112368514 | 4354 | 1: 112398275 | 4355 | 1: 112400081 | 4361 | 1: 112555088 |
| 4362 | 1: 112555094 | 4363 | 1: 112555107 | 4364 | 1: 112555108 | 4365 | 1: 112555300 | 4366 | 1: 112555322 |
| 4367 | 1: 112555340 | 4368 | 1: 112555460 | 4369 | 1: 112555492 | 4370 | 1: 112556677 | 4371 | 1: 112556716 |
| 4372 | 1: 112556776 | 4375 | 1: 112581875 | 4376 | 1: 112581930 | 4377 | 1: 112581975 | 4378 | 1: 112595293 |
| 4400 | 1: 113354543 | 4403 | 1: 113365903 | 4404 | 1: 113369018 | 4405 | 1: 113385595 | 4406 | 1: 113385624 |
| 4407 | 1: 113385658 | 4417 | 1: 113584302 | 4457 | 1: 114467455 | 4460 | 1: 114596976 | 4462 | 1: 114728742 |
| 4465 | 1: 114816068 | 4468 | 1: 114840022 | 4488 | 1: 115324305 | 4490 | 1: 115377104 | 4491 | 1: 115413995 |
| 4496 | 1: 115579666 | 4502 | 1: 115790600 | 4528 | 1: 116754887 | 4532 | 1: 116818074 | 4533 | 1: 116821719 |
| 4535 | 1: 116860895 | 4538 | 1: 116891857 | 4540 | 1: 117003117 | 4546 | 1: 117113278 | 4556 | 1: 117319749 |
| 4563 | 1: 117420923 | 4564 | 1: 117421250 | 4576 | 1: 117738265 | 4578 | 1: 117758868 | 4586 | 1: 117887255 |
| 4587 | 1: 117897860 | 4588 | 1: 117912537 | 4589 | 1: 117935755 | 4591 | 1: 117945817 | 4597 | 1: 118048415 |
| 4598 | 1: 118085158 | 4600 | 1: 118114537 | 4602 | 1: 118133789 | 4605 | 1: 118196612 | 4608 | 1: 118214654 |
| 4609 | 1: 118227302 | 4610 | 1: 118256496 | 4611 | 1: 118257517 | 4612 | 1: 118259606 | 4613 | 1: 118273499 |
| 4614 | 1: 118276261 | 4615 | 1: 118276735 | 4616 | 1: 118277267 | 4617 | 1: 118278387 | 4618 | 1: 118284875 |
| 4619 | 1: 118286166 | 4620 | 1: 118288018 | 4621 | 1: 118290177 | 4622 | 1: 118295131 | 4635 | 1: 118530178 |
| 4636 | 1: 118544527 | 4642 | 1: 118690300 | 4643 | 1: 118690309 | 4652 | 1: 118962324 | 4654 | 1: 118985100 |
| 4665 | 1: 119176913 | 4666 | 1: 119189605 | 4667 | 1: 119210631 | 4672 | 1: 119350100 | 4683 | 1: 119561515 |
| 4684 | 1: 119561537 | 4685 | 1: 119561564 | 4701 | 1: 120064732 | 4712 | 1: 120386879 | 4722 | 1: 120500835 |
| 4726 | 1: 120563845 | 4760 | 1: 121647921 | 4762 | 1: 121707542 | 4771 | 1: 121946641 | 4781 | 1: 122232742 |
| 4785 | 1: 122286938 | 4786 | 1: 122287045 | 4790 | 1: 122500672 | 4794 | 1: 122540031 | 4799 | 1: 122654668 |
| 4801 | 1: 122686036 | 4807 | 1: 122783659 | 4808 | 1: 122784649 | 4810 | 1: 122849903 | 4811 | 1: 122901551 |
| 4814 | 1: 123034294 | 4819 | 1: 123266689 | 4821 | 1: 123270508 | 4827 | 1: 123475726 | 4835 | 1: 123679492 |
| 4836 | 1: 123692405 | 4837 | 1: 123706359 | 4838 | 1: 123709988 | 4839 | 1: 123720581 | 4848 | 1: 124014834 |
| 4854 | 1: 124194081 | 4860 | 1: 124271732 | 4861 | 1: 124271768 | 4862 | 1: 124271786 | 4863 | 1: 124314731 |
| 4864 | 1: 124324829 | 4865 | 1: 124331752 | 4866 | 1: 124343187 | 4870 | 1: 124506907 | 4871 | 1: 124528147 |
| 4878 | 1: 124666898 | 4879 | 1: 124672817 | 4886 | 1: 124887464 | 4891 | 1: 124948606 | 4894 | 1: 124980393 |
| 4895 | 1: 124980562 | 4898 | 1: 125121982 | 4902 | 1: 125269301 | 4904 | 1: 125360929 | 4905 | 1: 125363719 |
| 4907 | 1: 125382225 | 4908 | 1: 125386885 | 4913 | 1: 125521147 | 4919 | 1: 125583210 | 4920 | 1: 125608898 |
| 4922 | 1: 125609041 | 4928 | 1: 125643614 | 4944 | 1: 125851156 | 4946 | 1: 125931085 | 4951 | 1: 126050865 |
| 4953 | 1: 126166588 | 4954 | 1: 126167017 | 4955 | 1: 126188011 | 4962 | 1: 126320901 | 4970 | 1: 126452685 |
| 4979 | 1: 126828386 | 4981 | 1: 126867108 | 4989 | 1: 127247333 | 4997 | 1: 127448104 | 5001 | 1: 127536751 |
| 5004 | 1: 127609705 | 5007 | 1: 127732186 | 5010 | 1: 127791769 | 5011 | 1: 127843966 | 5015 | 1: 127884865 |
| 5020 | 1: 127963540 | 5022 | 1: 128091201 | 5032 | 1: 128267812 | 5037 | 1: 128374395 | 5042 | 1: 128512081 |
| 5073 | 1: 129150388 | 5080 | 1: 129402104 | 5081 | 1: 129418328 | 5089 | 1: 129590492 | 5090 | 1: 129591720 |
| 5091 | 1: 129592529 | 5095 | 1: 129754416 | 5127 | 1: 130632012 | 5128 | 1: 130658736 | 5130 | 1: 130720354 |
| 5131 | 1: 130728433 | 5134 | 1: 130792978 | 5137 | 1: 130920062 | 5140 | 1: 130954555 | 5179 | 1: 131419104 |
| 5222 | 1: 131860577 | 5242 | 1: 132473942 | 5253 | 1: 132876761 | 5264 | 1: 133379843 | 5265 | 1: 133384149 |
| 5266 | 1: 133394080 | 5271 | 1: 133540327 | 5272 | 1: 133591491 | 5289 | 1: 134186323 | 5294 | 1: 134285791 |
| 5298 | 1: 134384145 | 5300 | 1: 134413818 | 5301 | 1: 134413821 | 5306 | 1: 134477738 | 5324 | 1: 134948789 |
| 5325 | 1: 134961346 | 5327 | 1: 134973908 | 5329 | 1: 135015189 | 5331 | 1: 135049866 | 5337 | 1: 135208575 |
| 5341 | 1: 135315701 | 5342 | 1: 135345248 | 5346 | 1: 135410192 | 5347 | 1: 135414049 | 5360 | 1: 135642886 |
| 5361 | 1: 135643955 | 5362 | 1: 135644811 | 5363 | 1: 135653473 | 5366 | 1: 135665914 | 5367 | 1: 135666200 |
| 5368 | 1: 135666237 | 5369 | 1: 135666283 | 5370 | 1: 135670574 | 5371 | 1: 135672004 | 5372 | 1: 135694251 |
| 5374 | 1: 135753894 | 5375 | 1: 135753940 | 5376 | 1: 135753983 | 5377 | 1: 135754044 | 5378 | 1: 135754264 |
| 5379 | 1: 135754300 | 5380 | 1: 135758284 | 5381 | 1: 135758290 | 5382 | 1: 135758332 | 5383 | 1: 135762033 |
| 5384 | 1: 135763704 | 5385 | 1: 135769522 | 5397 | 1: 135880893 | 5399 | 1: 135916803 | 5400 | 1: 135927623 |
| 5401 | 1: 135972509 | 5402 | 1: 135974364 | 5405 | 1: 136033754 | 5406 | 1: 136047835 | 5413 | 1: 136204362 |
| 5415 | 1: 136311162 | 5427 | 1: 136734614 | 5434 | 1: 137049621 | 5438 | 1: 137106364 | 5443 | 1: 137294127 |
| 5472 | 1: 138643152 | 5473 | 1: 138647040 | 5477 | 1: 138728014 | 5478 | 1: 138739021 | 5483 | 1: 138798912 |
| 5489 | 1: 138994500 | 5498 | 1: 139184610 | 5505 | 1: 139411806 | 5508 | 1: 139449113 | 5521 | 1: 139899458 |
| 5536 | 1: 140266541 | 5545 | 1: 140543741 | 5554 | 1: 140754935 | 5555 | 1: 140755072 | 5558 | 1: 140876675 |
| 5575 | 1: 141145402 | 5579 | 1: 141316735 | 5580 | 1: 141319127 | 5581 | 1: 141319994 | 5583 | 1: 141339227 |
| 5584 | 1: 141345591 | 5585 | 1: 141362575 | 5586 | 1: 141364352 | 5587 | 1: 141374294 | 5588 | 1: 141376997 |
| 5590 | 1: 141395382 | 5591 | 1: 141421683 | 5597 | 1: 141511318 | 5601 | 1: 141610105 | 5602 | 1: 141610924 |
| 5604 | 1: 141627871 | 5606 | 1: 141628909 | 5608 | 1: 141649600 | 5610 | 1: 141651362 | 5611 | 1: 141653130 |
| 5612 | 1: 141653134 | 5613 | 1: 141654271 | 5614 | 1: 141654547 | 5615 | 1: 141654551 | 5617 | 1: 141667906 |
| 5622 | 1: 141734030 | 5623 | 1: 141741068 | 5625 | 1: 141823289 | 5633 | 1: 142000532 | 5641 | 1: 142239516 |
| 5643 | 1: 142251697 | 5651 | 1: 142362891 | 5654 | 1: 142393561 | 5657 | 1: 142478032 | 5659 | 1: 142489032 |
| 5667 | 1: 142635123 | 5672 | 1: 142787751 | 5693 | 1: 143128593 | 5700 | 1: 143227118 | 5702 | 1: 143334405 |
| 5706 | 1: 143406113 | 5711 | 1: 143569447 | 5712 | 1: 143579707 | 5713 | 1: 143582830 | 5714 | 1: 143588692 |
| 5715 | 1: 143591196 | 5716 | 1: 143593029 | 5717 | 1: 143594628 | 5726 | 1: 143746215 | 5728 | 1: 143749139 |
| 5729 | 1: 143752954 | 5747 | 1: 144050118 | 5748 | 1: 144050120 | 5757 | 1: 144333701 | 5759 | 1: 144372951 |
| 5763 | 1: 144435905 | 5772 | 1: 144635480 | 5776 | 1: 144757986 | 5780 | 1: 144787067 | 5781 | 1: 144809797 |
| 5786 | 1: 144894365 | 5794 | 1: 145080672 | 5797 | 1: 145128073 | 5799 | 1: 145136789 | 5802 | 1: 145176107 |
| 5804 | 1: 145282436 | 5813 | 1: 145459919 | 5821 | 1: 145623145 | 5822 | 1: 145623795 | 5825 | 1: 145673098 |
| 5826 | 1: 145677709 | 5834 | 1: 145827286 | 5835 | 1: 145830272 | 5837 | 1: 145860541 | 5838 | 1: 145860568 |
| 5839 | 1: 145866834 | 5840 | 1: 145866900 | 5841 | 1: 145873941 | 5842 | 1: 145873979 | 5843 | 1: 145874030 |
| 5848 | 1: 145951072 | 5849 | 1: 145963833 | 5850 | 1: 145968413 | 5852 | 1: 145972525 | 5853 | 1: 145975945 |
| 5854 | 1: 145977236 | 5862 | 1: 146052159 | 5871 | 1: 146351589 | 5874 | 1: 146420270 | 5892 | 1: 146975928 |
| 5894 | 1: 147008926 | 5899 | 1: 147098097 | 5902 | 1: 147122843 | 5918 | 1: 147397092 | 5919 | 1: 147417101 |
| 5921 | 1: 147431978 | 5927 | 1: 147563989 | 5941 | 1: 147846791 | 5946 | 1: 147903408 | 5978 | 1: 148430252 |
| 5979 | 1: 148466864 | 5981 | 1: 148508063 | 5982 | 1: 148508133 | 5984 | 1: 148513206 | 5985 | 1: 148549091 |
| 5994 | 1: 148736098 | 6000 | 1: 148871697 | 6004 | 1: 148964961 | 6010 | 1: 149177892 | 6011 | 1: 149189606 |
| 6014 | 1: 149317573 | 6015 | 1: 149320003 | 6016 | 1: 149323616 | 6017 | 1: 149324955 | 6018 | 1: 149328203 |
| 6019 | 1: 149340128 | 6020 | 1: 149342542 | 6021 | 1: 149348963 | 6022 | 1: 149351545 | 6023 | 1: 149358100 |
| 6024 | 1: 149360084 | 6025 | 1: 149362014 | 6026 | 1: 149366388 | 6027 | 1: 149367120 | 6028 | 1: 149368535 |
| 6029 | 1: 149371783 | 6030 | 1: 149374604 | 6031 | 1: 149375645 | 6032 | 1: 149376574 | 6033 | 1: 149378550 |
| 6034 | 1: 149379934 | 6035 | 1: 149380878 | 6036 | 1: 149381954 | 6037 | 1: 149390100 | 6042 | 1: 149443669 |
| 6049 | 1: 149642436 | 6065 | 1: 149898235 | 6066 | 1: 149902664 | 6068 | 1: 149929034 | 6077 | 1: 150129027 |
| 6078 | 1: 150129035 | 6079 | 1: 150129044 | 6080 | 1: 150129048 | 6081 | 1: 150144069 | 6083 | 1: 150151876 |
| 6093 | 1: 150440522 | 6106 | 1: 150701995 | 6109 | 1: 150741379 | 6119 | 1: 151001062 | 6122 | 1: 151048846 |
| 6124 | 1: 151109660 | 6134 | 1: 151324257 | 6135 | 1: 151326467 | 6148 | 1: 151556745 | 6149 | 1: 151560943 |
| 6150 | 1: 151561380 | 6154 | 1: 151623827 | 6158 | 1: 151643959 | 6161 | 1: 151668710 | 6162 | 1: 151671521 |
| 6163 | 1: 151673956 | 6164 | 1: 151675356 | 6165 | 1: 151677189 | 6182 | 1: 151922660 | 6186 | 1: 152015544 |
| 6189 | 1: 152071824 | 6215 | 1: 152347862 | 6235 | 1: 152417708 | 6237 | 1: 152422584 | 6238 | 1: 152428562 |
| 6249 | 1: 152632298 | 6252 | 1: 152678335 | 6254 | 1: 152745164 | 6260 | 1: 152826141 | 6279 | 1: 153250410 |
| 6301 | 1: 153688732 | 6308 | 1: 153828051 | 6322 | 1: 154004623 | 6323 | 1: 154009064 | 6324 | 1: 154009137 |
| 6325 | 1: 154009145 | 6327 | 1: 154019636 | 6329 | 1: 154038751 | 6341 | 1: 154370113 | 6358 | 1: 154858681 |
| 6388 | 1: 155496781 | 6400 | 1: 155679617 | 6401 | 1: 155679654 | 6402 | 1: 155679691 | 6403 | 1: 155679758 |
| 6404 | 1: 155679762 | 6407 | 1: 155716904 | 6429 | 1: 156040352 | 6434 | 1: 156108198 | 6469 | 1: 156749778 |
| 6502 | 1: 157422591 | 6505 | 1: 157455223 | 6508 | 1: 157589021 | 6514 | 1: 157988428 | 6517 | 1: 158029386 |
| 6518 | 1: 158031759 | 6521 | 1: 158140847 | 6522 | 1: 158227949 | 6550 | 2: 773366 | 6570 | 2: 1116337 |
| 6571 | 2: 1120855 | 6576 | 2: 1174595 | 6579 | 2: 1234786 | 6580 | 2: 1239635 | 6584 | 2: 1280967 |
| 6585 | 2: 1281046 | 6586 | 2: 1286624 | 6587 | 2: 1286642 | 6588 | 2: 1286747 | 6607 | 2: 1669475 |
| 6615 | 2: 1857324 | 6616 | 2: 1859648 | 6623 | 2: 1996597 | 6642 | 2: 2733310 | 6649 | 2: 2936048 |
| 6657 | 2: 3205071 | 6671 | 2: 3644419 | 6679 | 2: 3866542 | 6680 | 2: 3866584 | 6688 | 2: 4033904 |
| 6692 | 2: 4056030 | 6693 | 2: 4056116 | 6702 | 2: 4244343 | 6705 | 2: 4311599 | 6706 | 2: 4311602 |
| 6707 | 2: 4311756 | 6708 | 2: 4318724 | 6710 | 2: 4360783 | 6719 | 2: 4631707 | 6720 | 2: 4634839 |
| 6721 | 2: 4634862 | 6722 | 2: 4634886 | 6723 | 2: 4634902 | 6724 | 2: 4634989 | 6725 | 2: 4655242 |
| 6727 | 2: 4687767 | 6731 | 2: 4782801 | 6749 | 2: 5105483 | 6754 | 2: 5312133 | 6757 | 2: 5343162 |
| 6760 | 2: 5372449 | 6767 | 2: 5418497 | 6770 | 2: 5433956 | 6771 | 2: 5437701 | 6776 | 2: 5564286 |
| 6778 | 2: 5598208 | 6780 | 2: 5605765 | 6781 | 2: 5668859 | 6786 | 2: 5824441 | 6806 | 2: 6219961 |
| 6807 | 2: 6220338 | 6820 | 2: 6413902 | 6826 | 2: 6665981 | 6827 | 2: 6670529 | 6828 | 2: 6670740 |
| 6829 | 2: 6670761 | 6830 | 2: 6670813 | 6831 | 2: 6670846 | 6842 | 2: 6830098 | 6844 | 2: 6891780 |
| 6846 | 2: 6946227 | 6851 | 2: 7030426 | 6852 | 2: 7053225 | 6855 | 2: 7145809 | 6859 | 2: 7201484 |
| 6860 | 2: 7214741 | 6861 | 2: 7228554 | 6865 | 2: 7278045 | 6867 | 2: 7321689 | 6869 | 2: 7334093 |
| 6870 | 2: 7339046 | 6871 | 2: 7346647 | 6874 | 2: 7389988 | 6876 | 2: 7414723 | 6877 | 2: 7487693 |
| 6880 | 2: 7550253 | 6893 | 2: 7824299 | 6894 | 2: 7854802 | 6895 | 2: 7854894 | 6896 | 2: 7854898 |
| 6903 | 2: 8149483 | 6907 | 2: 8268124 | 6909 | 2: 8273877 | 6925 | 2: 8874962 | 6926 | 2: 8891833 |
| 6948 | 2: 9583773 | 6949 | 2: 9583875 | 6957 | 2: 9791102 | 6960 | 2: 9831264 | 6985 | 2: 10595947 |
| 6986 | 2: 10596946 | 6987 | 2: 10597759 | 6988 | 2: 10603551 | 7032 | 2: 12051677 | 7037 | 2: 12305668 |
| 7043 | 2: 12489693 | 7058 | 2: 13087374 | 7071 | 2: 13360171 | 7072 | 2: 13382039 | 7082 | 2: 13695930 |
| 7084 | 2: 13709033 | 7086 | 2: 13779018 | 7088 | 2: 13797995 | 7090 | 2: 13822009 | 7091 | 2: 13823524 |
| 7092 | 2: 13862800 | 7102 | 2: 14216227 | 7127 | 2: 14784977 | 7139 | 2: 15119742 | 7147 | 2: 15329884 |
| 7157 | 2: 15549082 | 7162 | 2: 15690371 | 7163 | 2: 15693009 | 7164 | 2: 15696748 | 7168 | 2: 15821266 |
| 7173 | 2: 15967374 | 7190 | 2: 16339174 | 7207 | 2: 16790984 | 7211 | 2: 16932422 | 7217 | 2: 17092308 |
| 7223 | 2: 17311769 | 7236 | 2: 17583628 | 7241 | 2: 17659987 | 7242 | 2: 17735524 | 7246 | 2: 17833404 |
| 7250 | 2: 17967333 | 7254 | 2: 18038068 | 7260 | 2: 18147142 | 7261 | 2: 18165858 | 7263 | 2: 18229834 |
| 7264 | 2: 18229859 | 7266 | 2: 18241132 | 7268 | 2: 18244789 | 7270 | 2: 18253097 | 7271 | 2: 18254488 |
| 7273 | 2: 18262337 | 7275 | 2: 18270755 | 7276 | 2: 18271356 | 7277 | 2: 18271358 | 7278 | 2: 18271418 |
| 7279 | 2: 18272565 | 7280 | 2: 18272965 | 7281 | 2: 18275874 | 7282 | 2: 18277584 | 7283 | 2: 18277939 |
| 7284 | 2: 18280757 | 7285 | 2: 18280781 | 7286 | 2: 18282254 | 7287 | 2: 18282552 | 7288 | 2: 18290509 |
| 7289 | 2: 18291940 | 7290 | 2: 18307699 | 7291 | 2: 18323628 | 7293 | 2: 18378637 | 7297 | 2: 18502293 |
| 7304 | 2: 18586886 | 7305 | 2: 18595133 | 7306 | 2: 18605578 | 7310 | 2: 18642606 | 7314 | 2: 18778867 |
| 7316 | 2: 18866785 | 7317 | 2: 18869734 | 7325 | 2: 18981323 | 7326 | 2: 18986957 | 7330 | 2: 19045985 |
| 7335 | 2: 19136987 | 7346 | 2: 19340327 | 7352 | 2: 19534377 | 7355 | 2: 19593435 | 7356 | 2: 19626883 |
| 7357 | 2: 19648311 | 7358 | 2: 19648602 | 7368 | 2: 19897160 | 7369 | 2: 19918403 | 7378 | 2: 20155806 |
| 7380 | 2: 20264485 | 7383 | 2: 20344434 | 7387 | 2: 20427608 | 7394 | 2: 20674260 | 7401 | 2: 20845761 |
| 7403 | 2: 20861585 | 7407 | 2: 21064906 | 7421 | 2: 21401355 | 7422 | 2: 21405561 | 7428 | 2: 21553608 |
| 7438 | 2: 21688312 | 7443 | 2: 21849771 | 7444 | 2: 21867557 | 7445 | 2: 21867592 | 7447 | 2: 21914587 |
| 7449 | 2: 21967376 | 7451 | 2: 22019700 | 7452 | 2: 22051754 | 7458 | 2: 22154085 | 7466 | 2: 22396788 |
| 7472 | 2: 22599197 | 7474 | 2: 22608474 | 7479 | 2: 22767655 | 7480 | 2: 22787231 | 7485 | 2: 23032717 |
| 7489 | 2: 23063150 | 7496 | 2: 23295824 | 7497 | 2: 23312807 | 7502 | 2: 23459486 | 7504 | 2: 23476574 |
| 7505 | 2: 23481265 | 7506 | 2: 23481554 | 7507 | 2: 23481572 | 7509 | 2: 23487782 | 7510 | 2: 23501169 |
| 7511 | 2: 23537225 | 7512 | 2: 23557793 | 7513 | 2: 23558570 | 7520 | 2: 23643567 | 7522 | 2: 23666640 |
| 7523 | 2: 23676088 | 7525 | 2: 23691155 | 7526 | 2: 23696931 | 7527 | 2: 23699667 | 7534 | 2: 23962288 |
| 7536 | 2: 23971606 | 7545 | 2: 24172853 | 7546 | 2: 24176608 | 7552 | 2: 24299477 | 7554 | 2: 24320827 |
| 7555 | 2: 24320845 | 7575 | 2: 24669527 | 7586 | 2: 24895083 | 7591 | 2: 25095591 | 7595 | 2: 25243017 |
| 7597 | 2: 25329638 | 7600 | 2: 25433245 | 7601 | 2: 25441115 | 7602 | 2: 25443814 | 7606 | 2: 25523128 |
| 7607 | 2: 25531340 | 7608 | 2: 25539975 | 7609 | 2: 25556600 | 7617 | 2: 25649108 | 7620 | 2: 25710237 |
| 7658 | 2: 26534630 | 7667 | 2: 26760780 | 7669 | 2: 26762215 | 7670 | 2: 26771611 | 7671 | 2: 26774438 |
| 7674 | 2: 26851479 | 7691 | 2: 27096366 | 7698 | 2: 27312219 | 7702 | 2: 27432834 | 7727 | 2: 27915981 |
| 7736 | 2: 28189986 | 7738 | 2: 28287714 | 7739 | 2: 28287763 | 7740 | 2: 28287812 | 7741 | 2: 28287836 |
| 7746 | 2: 28373427 | 7749 | 2: 28418211 | 7764 | 2: 28631973 | 7780 | 2: 28937935 | 7813 | 2: 29825101 |
| 7817 | 2: 29924141 | 7825 | 2: 30218788 | 7826 | 2: 30225126 | 7827 | 2: 30235162 | 7828 | 2: 30235271 |
| 7837 | 2: 30489470 | 7840 | 2: 30505625 | 7847 | 2: 30620848 | 7850 | 2: 30714513 | 7852 | 2: 30729817 |
| 7867 | 2: 31118343 | 7868 | 2: 31118369 | 7869 | 2: 31121656 | 7887 | 2: 31621372 | 7905 | 2: 32163209 |
| 7921 | 2: 32683358 | 7923 | 2: 32754895 | 7930 | 2: 33004780 | 7937 | 2: 33199953 | 7948 | 2: 33575101 |
| 7949 | 2: 33575122 | 7950 | 2: 33654382 | 7952 | 2: 33671969 | 7953 | 2: 33671986 | 7954 | 2: 33694528 |
| 7959 | 2: 33883065 | 7962 | 2: 33960798 | 7978 | 2: 34316478 | 7981 | 2: 34353723 | 7986 | 2: 34439099 |
| 7989 | 2: 34507044 | 8027 | 2: 35881554 | 8035 | 2: 36123841 | 8036 | 2: 36138834 | 8037 | 2: 36147200 |
| 8051 | 2: 36510607 | 8055 | 2: 36554095 | 8056 | 2: 36613946 | 8057 | 2: 36621379 | 8060 | 2: 36742166 |
| 8075 | 2: 37300847 | 8101 | 2: 38053061 | 8107 | 2: 38237985 | 8108 | 2: 38238121 | 8109 | 2: 38238372 |
| 8110 | 2: 38238415 | 8111 | 2: 38238441 | 8112 | 2: 38238520 | 8113 | 2: 38238541 | 8117 | 2: 38310384 |
| 8121 | 2: 38442344 | 8123 | 2: 38465755 | 8130 | 2: 38600295 | 8131 | 2: 38634299 | 8136 | 2: 38811572 |
| 8163 | 2: 39460697 | 8203 | 2: 40835843 | 8205 | 2: 40867544 | 8206 | 2: 40867584 | 8207 | 2: 40867585 |
| 8208 | 2: 40867624 | 8209 | 2: 40867647 | 8210 | 2: 40867669 | 8211 | 2: 40867710 | 8212 | 2: 40867713 |
| 8215 | 2: 41050502 | 8216 | 2: 41050562 | 8217 | 2: 41050564 | 8218 | 2: 41050613 | 8226 | 2: 41315483 |
| 8227 | 2: 41315504 | 8245 | 2: 41559277 | 8253 | 2: 41775609 | 8254 | 2: 41797511 | 8287 | 2: 42520826 |
| 8288 | 2: 42535114 | 8307 | 2: 43006124 | 8321 | 2: 43491727 | 8323 | 2: 43580317 | 8324 | 2: 43631101 |
| 8328 | 2: 43711459 | 8332 | 2: 43813071 | 8341 | 2: 44152000 | 8343 | 2: 44193529 | 8350 | 2: 44379204 |
| 8351 | 2: 44393093 | 8352 | 2: 44393964 | 8353 | 2: 44403432 | 8356 | 2: 44515259 | 8357 | 2: 44545955 |
| 8358 | 2: 44569313 | 8360 | 2: 44615292 | 8361 | 2: 44621241 | 8367 | 2: 44858072 | 8384 | 2: 45372211 |
| 8388 | 2: 45474859 | 8412 | 2: 45983105 | 8415 | 2: 46033456 | 8420 | 2: 46147458 | 8428 | 2: 46391874 |
| 8434 | 2: 46437983 | 8436 | 2: 46446081 | 8440 | 2: 46534111 | 8446 | 2: 46705643 | 8464 | 2: 47013353 |
| 8467 | 2: 47100164 | 8481 | 2: 47433064 | 8486 | 2: 47596275 | 8491 | 2: 47706783 | 8509 | 2: 48215370 |
| 8522 | 2: 48603698 | 8523 | 2: 48604345 | 8526 | 2: 48755071 | 8528 | 2: 48783617 | 8531 | 2: 48830819 |
| 8534 | 2: 48894232 | 8539 | 2: 48987003 | 8544 | 2: 49067927 | 8555 | 2: 49577101 | 8571 | 2: 50088797 |
| 8590 | 2: 50645993 | 8595 | 2: 50810980 | 8605 | 2: 51033258 | 8615 | 2: 51338525 | 8626 | 2: 51770242 |
| 8628 | 2: 51813793 | 8649 | 2: 52303945 | 8655 | 2: 52440698 | 8660 | 2: 52515449 | 8671 | 2: 52834508 |
| 8675 | 2: 52903006 | 8676 | 2: 52906421 | 8677 | 2: 52909824 | 8681 | 2: 52959066 | 8690 | 2: 53226738 |
| 8735 | 2: 54491736 | 8753 | 2: 54901841 | 8756 | 2: 54993038 | 8757 | 2: 54993081 | 8758 | 2: 54993084 |
| 8759 | 2: 54993089 | 8761 | 2: 55019715 | 8769 | 2: 55150342 | 8770 | 2: 55235366 | 8800 | 2: 56234809 |
| 8802 | 2: 56280951 | 8804 | 2: 56314003 | 8809 | 2: 56469340 | 8816 | 2: 56646269 | 8838 | 2: 57404778 |
| 8839 | 2: 57430549 | 8849 | 2: 57666386 | 8851 | 2: 57700250 | 8858 | 2: 57837920 | 8888 | 2: 58761910 |
| 8890 | 2: 58778117 | 8892 | 2: 58793131 | 8897 | 2: 58929038 | 8899 | 2: 58940947 | 8901 | 2: 58993535 |
| 8903 | 2: 59042027 | 8904 | 2: 59079693 | 8908 | 2: 59202049 | 8909 | 2: 59212187 | 8923 | 2: 59601537 |
| 8931 | 2: 59858246 | 8932 | 2: 59858284 | 8933 | 2: 59858319 | 8934 | 2: 59858339 | 8949 | 2: 60150792 |
| 8967 | 2: 60751699 | 8978 | 2: 61108555 | 8980 | 2: 61144456 | 8981 | 2: 61170888 | 8982 | 2: 61172063 |
| 8985 | 2: 61252370 | 8992 | 2: 61343961 | 8994 | 2: 61449061 | 8995 | 2: 61490160 | 8996 | 2: 61490261 |
| 8998 | 2: 61494582 | 9000 | 2: 61535058 | 9001 | 2: 61535147 | 9002 | 2: 61535149 | 9003 | 2: 61535213 |
| 9013 | 2: 61843883 | 9020 | 2: 62046115 | 9024 | 2: 62302155 | 9046 | 2: 62786824 | 9051 | 2: 62910878 |
| 9052 | 2: 62944872 | 9053 | 2: 62944925 | 9055 | 2: 63002162 | 9065 | 2: 63241642 | 9070 | 2: 63325322 |
| 9078 | 2: 63563631 | 9081 | 2: 63616590 | 9090 | 2: 63926681 | 9124 | 2: 64713810 | 9129 | 2: 64820071 |
| 9148 | 2: 65218951 | 9164 | 2: 65726104 | 9221 | 2: 67528810 | 9226 | 2: 67669505 | 9236 | 2: 68034573 |
| 9267 | 2: 69100324 | 9283 | 2: 69720593 | 9294 | 2: 70008307 | 9303 | 2: 70294251 | 9304 | 2: 70313073 |
| 9310 | 2: 70440178 | 9324 | 2: 70768201 | 9329 | 2: 70931242 | 9334 | 2: 71041850 | 9335 | 2: 71042132 |
| 9336 | 2: 71042190 | 9337 | 2: 71044225 | 9338 | 2: 71095900 | 9341 | 2: 71203819 | 9342 | 2: 71205559 |
| 9349 | 2: 71337903 | 9357 | 2: 71501065 | 9366 | 2: 71912340 | 9375 | 2: 72314326 | 9392 | 2: 72649795 |
| 9393 | 2: 72669915 | 9394 | 2: 72669943 | 9403 | 2: 72918814 | 9404 | 2: 72977278 | 9405 | 2: 72995902 |
| 9407 | 2: 73090036 | 9413 | 2: 73157226 | 9414 | 2: 73160964 | 9415 | 2: 73161983 | 9420 | 2: 73383708 |
| 9422 | 2: 73398069 | 9424 | 2: 73477830 | 9434 | 2: 73757540 | 9437 | 2: 73811343 | 9446 | 2: 73945664 |
| 9449 | 2: 74083815 | 9468 | 2: 74707536 | 9469 | 2: 74707559 | 9470 | 2: 74707612 | 9471 | 2: 74707658 |
| 9472 | 2: 74707710 | 9482 | 2: 75029605 | 9485 | 2: 75173951 | 9493 | 2: 75482167 | 9494 | 2: 75483513 |
| 9501 | 2: 75692826 | 9507 | 2: 75820235 | 9508 | 2: 75834257 | 9517 | 2: 76002206 | 9519 | 2: 76048349 |
| 9527 | 2: 76266636 | 9537 | 2: 76475875 | 9539 | 2: 76503911 | 9556 | 2: 77018992 | 9561 | 2: 77216172 |
| 9563 | 2: 77216223 | 9570 | 2: 77393386 | 9571 | 2: 77423386 | 9575 | 2: 77530544 | 9579 | 2: 77676860 |
| 9580 | 2: 77680269 | 9581 | 2: 77690134 | 9583 | 2: 77796311 | 9590 | 2: 77930065 | 9591 | 2: 77960137 |
| 9592 | 2: 77960148 | 9593 | 2: 77960205 | 9594 | 2: 77960221 | 9595 | 2: 78058933 | 9599 | 2: 78093605 |
| 9608 | 2: 78475004 | 9610 | 2: 78576254 | 9621 | 2: 78787744 | 9622 | 2: 78787785 | 9623 | 2: 78793670 |
| 9625 | 2: 78828241 | 9627 | 2: 78856439 | 9631 | 2: 78919311 | 9632 | 2: 78922277 | 9634 | 2: 78933501 |
| 9635 | 2: 78933506 | 9636 | 2: 78940906 | 9637 | 2: 78942899 | 9640 | 2: 79034805 | 9643 | 2: 79043516 |
| 9665 | 2: 79805247 | 9673 | 2: 79959399 | 9683 | 2: 80095415 | 9686 | 2: 80170719 | 9762 | 2: 83630529 |
| 9763 | 2: 83648977 | 9764 | 2: 83654198 | 9765 | 2: 83657997 | 9766 | 2: 83660404 | 9767 | 2: 83664117 |
| 9768 | 2: 83664826 | 9769 | 2: 83665834 | 9770 | 2: 83667393 | 9771 | 2: 83669899 | 9772 | 2: 83671239 |
| 9774 | 2: 83694268 | 9775 | 2: 83694872 | 9776 | 2: 83696501 | 9777 | 2: 83697095 | 9778 | 2: 83701382 |
| 9779 | 2: 83703221 | 9780 | 2: 83710452 | 9796 | 2: 84314672 | 9801 | 2: 84413080 | 9806 | 2: 84544851 |
| 9807 | 2: 84554472 | 9808 | 2: 84556946 | 9809 | 2: 84558765 | 9810 | 2: 84558813 | 9811 | 2: 84579866 |
| 9812 | 2: 84579884 | 9814 | 2: 84594590 | 9815 | 2: 84594621 | 9816 | 2: 84631977 | 9821 | 2: 84747533 |
| 9826 | 2: 84895919 | 9829 | 2: 84986688 | 9841 | 2: 85268807 | 9842 | 2: 85302489 | 9857 | 2: 85748841 |
| 9868 | 2: 85921857 | 9870 | 2: 85984791 | 9871 | 2: 86008072 | 9876 | 2: 86150518 | 9881 | 2: 86310570 |
| 9882 | 2: 86387502 | 9886 | 2: 86419348 | 9889 | 2: 86436688 | 9891 | 2: 86469285 | 9894 | 2: 86509868 |
| 9961 | 2: 88510673 | 9965 | 2: 88623312 | 9970 | 2: 88707441 | 9971 | 2: 88719162 | 9973 | 2: 88746032 |
| 9975 | 2: 88782810 | 9977 | 2: 88859157 | 9983 | 2: 89070767 | 9984 | 2: 89076240 | 9985 | 2: 89084301 |
| 9986 | 2: 89086388 | 9987 | 2: 89095014 | 9988 | 2: 89100826 | 9989 | 2: 89104328 | 9990 | 2: 89109055 |
| 9991 | 2: 89114149 | 9992 | 2: 89117889 | 9993 | 2: 89119778 | 9994 | 2: 89122696 | 9995 | 2: 89126698 |
| 9996 | 2: 89128778 | 9997 | 2: 89129756 | 9998 | 2: 89130672 | 9999 | 2: 89131642 | 10000 | 2: 89132131 |
| 10001 | 2: 89132635 | 10002 | 2: 89133746 | 10003 | 2: 89136358 | 10004 | 2: 89136948 | 10005 | 2: 89143985 |
| 10006 | 2: 89150132 | 10007 | 2: 89157341 | 10008 | 2: 89161844 | 10009 | 2: 89164769 | 10010 | 2: 89172177 |
| 10011 | 2: 89183562 | 10012 | 2: 89188097 | 10013 | 2: 89192815 | 10014 | 2: 89196778 | 10015 | 2: 89206007 |
| 10016 | 2: 89210274 | 10017 | 2: 89220562 | 10021 | 2: 89293059 | 10022 | 2: 89329459 | 10023 | 2: 89330715 |
| 10024 | 2: 89332510 | 10025 | 2: 89333883 | 10026 | 2: 89335072 | 10028 | 2: 89337942 | 10031 | 2: 89377233 |
| 10034 | 2: 89445178 | 10049 | 2: 89808466 | 10050 | 2: 89813015 | 10052 | 2: 89841096 | 10054 | 2: 89876885 |
| 10055 | 2: 89878690 | 10057 | 2: 89908253 | 10059 | 2: 89916678 | 10060 | 2: 89948655 | 10061 | 2: 89952700 |
| 10062 | 2: 89952808 | 10063 | 2: 89953249 | 10064 | 2: 89964009 | 10065 | 2: 89964459 | 10066 | 2: 89964908 |
| 10100 | 2: 91309327 | 10101 | 2: 91319564 | 10123 | 2: 92012717 | 10131 | 2: 92095096 | 10132 | 2: 92109122 |
| 10142 | 2: 92406522 | 10143 | 2: 92416012 | 10146 | 2: 92501151 | 10148 | 2: 92504237 | 10154 | 2: 92593249 |
| 10157 | 2: 92677123 | 10158 | 2: 92688749 | 10163 | 2: 92792925 | 10165 | 2: 92800370 | 10167 | 2: 92859552 |
| 10168 | 2: 92875951 | 10171 | 2: 93007619 | 10197 | 2: 93432778 | 10217 | 2: 93888418 | 10231 | 2: 94155809 |
| 10233 | 2: 94177314 | 10234 | 2: 94238285 | 10238 | 2: 94284858 | 10242 | 2: 94408041 | 10244 | 2: 94532812 |
| 10250 | 2: 94662652 | 10263 | 2: 94943212 | 10269 | 2: 95104701 | 10273 | 2: 95191751 | 10276 | 2: 95262942 |
| 10277 | 2: 95265410 | 10297 | 2: 95781931 | 10298 | 2: 95788314 | 10299 | 2: 95797570 | 10304 | 2: 95957388 |
| 10307 | 2: 96027434 | 10312 | 2: 96139223 | 10313 | 2: 96162249 | 10321 | 2: 96318089 | 10324 | 2: 96352046 |
| 10330 | 2: 96463623 | 10332 | 2: 96505449 | 10333 | 2: 96512869 | 10334 | 2: 96514006 | 10335 | 2: 96514035 |
| 10336 | 2: 96514066 | 10337 | 2: 96514071 | 10339 | 2: 96516949 | 10340 | 2: 96517000 | 10341 | 2: 96518404 |
| 10342 | 2: 96519726 | 10343 | 2: 96519764 | 10344 | 2: 96519823 | 10345 | 2: 96522707 | 10346 | 2: 96522837 |
| 10352 | 2: 96721652 | 10373 | 2: 97569993 | 10393 | 2: 98098074 | 10394 | 2: 98098574 | 10397 | 2: 98189476 |
| 10411 | 2: 98434332 | 10435 | 2: 99027387 | 10443 | 2: 99309195 | 10444 | 2: 99310709 | 10477 | 2: 100060201 |
| 10486 | 2: 100359058 | 10488 | 2: 100436598 | 10489 | 2: 100436640 | 10490 | 2: 100507665 | 10491 | 2: 100527030 |
| 10508 | 2: 100969194 | 10512 | 2: 101142510 | 10515 | 2: 101176628 | 10516 | 2: 101267741 | 10522 | 2: 101470754 |
| 10524 | 2: 101503694 | 10526 | 2: 101544902 | 10529 | 2: 101696463 | 10540 | 2: 102030097 | 10541 | 2: 102031180 |
| 10567 | 2: 102763970 | 10569 | 2: 102854361 | 10570 | 2: 102861535 | 10574 | 2: 103021453 | 10575 | 2: 103022409 |
| 10577 | 2: 103050047 | 10578 | 2: 103087349 | 10588 | 2: 103396204 | 10589 | 2: 103402752 | 10592 | 2: 103445587 |
| 10595 | 2: 103517827 | 10598 | 2: 103593199 | 10610 | 2: 103884686 | 10615 | 2: 104008673 | 10622 | 2: 104115856 |
| 10628 | 2: 104308034 | 10635 | 2: 104472870 | 10641 | 2: 104678498 | 10643 | 2: 104790859 | 10644 | 2: 104814826 |
| 10646 | 2: 104850637 | 10647 | 2: 104882698 | 10660 | 2: 105042627 | 10669 | 2: 105160301 | 10679 | 2: 105493554 |
| 10682 | 2: 105532073 | 10684 | 2: 105558282 | 10685 | 2: 105589302 | 10689 | 2: 105726409 | 10697 | 2: 105850938 |
| 10698 | 2: 105883087 | 10702 | 2: 105943923 | 10704 | 2: 106005198 | 10705 | 2: 106040867 | 10708 | 2: 106093074 |
| 10709 | 2: 106134401 | 10716 | 2: 106279517 | 10718 | 2: 106396433 | 10719 | 2: 106446238 | 10720 | 2: 106455619 |
| 10722 | 2: 106582615 | 10724 | 2: 106599852 | 10725 | 2: 106602909 | 10726 | 2: 106605195 | 10727 | 2: 106609300 |
| 10728 | 2: 106609491 | 10729 | 2: 106615459 | 10730 | 2: 106624131 | 10731 | 2: 106632743 | 10732 | 2: 106635677 |
| 10740 | 2: 106788337 | 10748 | 2: 106865812 | 10750 | 2: 106960370 | 10752 | 2: 107059040 | 10753 | 2: 107076804 |
| 10766 | 2: 107558581 | 10768 | 2: 107560640 | 10769 | 2: 107560655 | 10773 | 2: 107620491 | 10774 | 2: 107628799 |
| 10777 | 2: 107692575 | 10780 | 2: 107718206 | 10793 | 2: 107772997 | 10796 | 2: 107824497 | 10797 | 2: 107827321 |
| 10799 | 2: 107881110 | 10808 | 2: 108144100 | 10810 | 2: 108165798 | 10820 | 2: 108449215 | 10827 | 2: 108649909 |
| 10835 | 2: 108855822 | 10838 | 2: 108918709 | 10839 | 2: 108932264 | 10841 | 2: 108944061 | 10850 | 2: 109135684 |
| 10861 | 2: 109403406 | 10862 | 2: 109415989 | 10867 | 2: 109507997 | 10868 | 2: 109525518 | 10871 | 2: 109605505 |
| 10874 | 2: 109627420 | 10878 | 2: 109678083 | 10896 | 2: 110207989 | 10909 | 2: 110556883 | 10919 | 2: 111069391 |
| 10923 | 2: 111211200 | 10924 | 2: 111216842 | 10931 | 2: 111473451 | 10946 | 2: 111903128 | 10950 | 2: 112002693 |
| 10951 | 2: 112064672 | 10952 | 2: 112068968 | 10963 | 2: 112309161 | 10982 | 2: 112824303 | 10985 | 2: 112865726 |
| 10988 | 2: 112884446 | 10996 | 2: 112999914 | 10998 | 2: 113039809 | 11000 | 2: 113071684 | 11004 | 2: 113217166 |
| 11005 | 2: 113217179 | 11006 | 2: 113217208 | 11007 | 2: 113217254 | 11033 | 2: 113775291 | 11040 | 2: 113927720 |
| 11052 | 2: 114312086 | 11056 | 2: 114441340 | 11057 | 2: 114511997 | 11070 | 2: 114795811 | 11072 | 2: 114857637 |
| 11087 | 2: 115277541 | 11088 | 2: 115279258 | 11092 | 2: 115316715 | 11096 | 2: 115405473 | 11097 | 2: 115455721 |
| 11106 | 2: 115621193 | 11108 | 2: 115625826 | 11125 | 2: 115953383 | 11155 | 2: 116733465 | 11161 | 2: 116888348 |
| 11167 | 2: 117033411 | 11168 | 2: 117047832 | 11169 | 2: 117079799 | 11172 | 2: 117133057 | 11178 | 2: 117240992 |
| 11187 | 2: 117466579 | 11195 | 2: 117945652 | 11200 | 2: 118099830 | 11207 | 2: 118201421 | 11220 | 2: 118509097 |
| 11222 | 2: 118554241 | 11239 | 2: 119145772 | 11240 | 2: 119196348 | 11241 | 2: 119198191 | 11242 | 2: 119199613 |
| 11244 | 2: 119244130 | 11245 | 2: 119264906 | 11248 | 2: 119291176 | 11252 | 2: 119322052 | 11257 | 2: 119465613 |
| 11260 | 2: 119511934 | 11261 | 2: 119512418 | 11262 | 2: 119522741 | 11263 | 2: 119526528 | 11264 | 2: 119533194 |
| 11267 | 2: 119548536 | 11269 | 2: 119562713 | 11270 | 2: 119564586 | 11271 | 2: 119565180 | 11272 | 2: 119566000 |
| 11273 | 2: 119576736 | 11277 | 2: 119825482 | 11278 | 2: 119862058 | 11290 | 2: 120178985 | 11292 | 2: 120195958 |
| 11293 | 2: 120199797 | 11308 | 2: 120568141 | 11310 | 2: 120671914 | 11319 | 2: 120900260 | 11322 | 2: 120932791 |
| 11331 | 2: 121151913 | 11333 | 2: 121188721 | 11352 | 2: 121755672 | 11358 | 2: 121893423 | 11359 | 2: 121894763 |
| 11375 | 2: 122580635 | 11382 | 2: 122800513 | 11385 | 2: 122878392 | 11393 | 2: 123070777 | 11394 | 2: 123070905 |
| 11395 | 2: 123070944 | 11396 | 2: 123100345 | 11397 | 2: 123100370 | 11414 | 2: 123539540 | 11415 | 2: 123539541 |
| 11417 | 2: 123562349 | 11419 | 2: 123590694 | 11420 | 2: 123590830 | 11421 | 2: 123610591 | 11427 | 2: 123692694 |
| 11447 | 2: 124332744 | 11453 | 2: 124600152 | 11458 | 2: 124710998 | 11462 | 2: 124913543 | 11470 | 2: 125166728 |
| 11476 | 2: 125250396 | 11479 | 2: 125277549 | 11480 | 2: 125286027 | 11481 | 2: 125349483 | 11490 | 2: 125625209 |
| 11492 | 2: 125653640 | 11493 | 2: 125653952 | 11496 | 2: 125742333 | 11499 | 2: 125810128 | 11502 | 2: 125931055 |
| 11503 | 2: 125952736 | 11508 | 2: 126114829 | 11511 | 2: 126173762 | 11515 | 2: 126267198 | 11516 | 2: 126267881 |
| 11517 | 2: 126273576 | 11518 | 2: 126275683 | 11519 | 2: 126286517 | 11522 | 2: 126366193 | 11523 | 2: 126367550 |
| 11525 | 2: 126372344 | 11526 | 2: 126380236 | 11529 | 2: 126489135 | 11575 | 2: 127003970 | 11576 | 2: 127004702 |
| 11583 | 2: 127018411 | 11587 | 2: 127027686 | 11589 | 2: 127038523 | 11606 | 2: 127110467 | 11613 | 2: 127238953 |
| 11623 | 2: 127397394 | 11629 | 2: 127525976 | 11634 | 2: 127633593 | 11636 | 2: 127635918 | 11637 | 2: 127639135 |
| 11654 | 2: 128049090 | 11656 | 2: 128099391 | 11672 | 2: 128508443 | 11673 | 2: 128508479 | 11677 | 2: 128603208 |
| 11683 | 2: 128718168 | 11688 | 2: 128782288 | 11691 | 2: 128791601 | 11692 | 2: 128804408 | 11695 | 2: 128916878 |
| 11701 | 2: 129068395 | 11702 | 2: 129073892 | 11704 | 2: 129084385 | 11705 | 2: 129095315 | 11706 | 2: 129098350 |
| 11707 | 2: 129101237 | 11719 | 2: 129414787 | 11721 | 2: 129451897 | 11722 | 2: 129453738 | 11723 | 2: 129463584 |
| 11724 | 2: 129475673 | 11725 | 2: 129514375 | 11735 | 2: 129823411 | 11740 | 2: 129952968 | 11750 | 2: 130179342 |
| 11751 | 2: 130179458 | 11753 | 2: 130297720 | 11759 | 2: 130405723 | 11761 | 2: 130416105 | 11771 | 2: 130763239 |
| 11773 | 2: 130778311 | 11775 | 2: 130786713 | 11777 | 2: 130887344 | 11779 | 2: 130922023 | 11780 | 2: 130922555 |
| 11781 | 2: 130924042 | 11786 | 2: 131056297 | 11788 | 2: 131116831 | 11792 | 2: 131182393 | 11793 | 2: 131183245 |
| 11799 | 2: 131239243 | 11810 | 2: 131495314 | 11812 | 2: 131522458 | 11818 | 2: 131759255 | 11827 | 2: 131997425 |
| 11828 | 2: 132032080 | 11829 | 2: 132052956 | 11836 | 2: 132152838 | 11850 | 2: 132304236 | 11856 | 2: 132453956 |
| 11862 | 2: 132630596 | 11873 | 2: 132828927 | 11874 | 2: 132829391 | 11883 | 2: 133050393 | 11884 | 2: 133069759 |
| 11886 | 2: 133077482 | 11888 | 2: 133094549 | 11889 | 2: 133095512 | 11892 | 2: 133207446 | 11897 | 2: 133305481 |
| 11901 | 2: 133364775 | 11905 | 2: 133421233 | 11908 | 2: 133536795 | 11910 | 2: 133542411 | 11915 | 2: 133623325 |
| 11923 | 2: 133694191 | 11927 | 2: 133778125 | 11928 | 2: 133778616 | 11929 | 2: 133785516 | 11931 | 2: 133786535 |
| 11932 | 2: 133787224 | 11933 | 2: 133787956 | 11934 | 2: 133787982 | 11935 | 2: 133788783 | 11957 | 2: 134106505 |
| 11963 | 2: 134189401 | 11964 | 2: 134189458 | 11965 | 2: 134190122 | 11966 | 2: 134190219 | 11977 | 2: 134376164 |
| 11979 | 2: 134438884 | 11982 | 2: 134480181 | 11983 | 2: 134480249 | 11984 | 2: 134480295 | 11985 | 2: 134480304 |
| 11988 | 2: 134508261 | 11997 | 2: 134616684 | 11998 | 2: 134616699 | 12008 | 2: 134803486 | 12012 | 2: 134834209 |
| 12013 | 2: 134843772 | 12019 | 2: 134917533 | 12020 | 2: 134917563 | 12021 | 2: 134917597 | 12022 | 2: 134917643 |
| 12023 | 2: 134917658 | 12031 | 2: 135091858 | 12033 | 2: 135100100 | 12035 | 2: 135114924 | 12038 | 2: 135166182 |
| 12039 | 2: 135171128 | 12042 | 2: 135233030 | 12043 | 2: 135235158 | 12048 | 2: 135319271 | 12054 | 2: 135454017 |
| 12058 | 2: 135514495 | 12063 | 2: 135598504 | 12064 | 2: 135598610 | 12066 | 2: 135604349 | 12069 | 2: 135646413 |
| 12074 | 2: 135709576 | 12076 | 2: 135727874 | 12077 | 2: 135727928 | 12079 | 2: 135837161 | 12080 | 2: 135837263 |
| 12082 | 2: 135865945 | 12083 | 2: 135887710 | 12084 | 2: 135900620 | 12085 | 2: 135900833 | 12086 | 2: 135904261 |
| 12087 | 2: 135908217 | 12091 | 2: 136093517 | 12092 | 2: 136093612 | 12104 | 3: 197807 | 12127 | 3: 746327 |
| 12135 | 3: 867632 | 12139 | 3: 923989 | 12140 | 3: 924352 | 12142 | 3: 941811 | 12146 | 3: 969297 |
| 12155 | 3: 1114174 | 12162 | 3: 1183922 | 12181 | 3: 1651460 | 12184 | 3: 1672587 | 12188 | 3: 1729118 |
| 12195 | 3: 1939081 | 12198 | 3: 1965533 | 12199 | 3: 1967307 | 12204 | 3: 2023241 | 12206 | 3: 2062805 |
| 12208 | 3: 2064509 | 12211 | 3: 2089682 | 12212 | 3: 2098556 | 12213 | 3: 2104449 | 12235 | 3: 2740113 |
| 12243 | 3: 2986445 | 12245 | 3: 2993307 | 12250 | 3: 3052396 | 12283 | 3: 3863731 | 12305 | 3: 4250424 |
| 12306 | 3: 4264560 | 12307 | 3: 4280470 | 12308 | 3: 4285570 | 12310 | 3: 4298685 | 12312 | 3: 4313521 |
| 12314 | 3: 4363949 | 12316 | 3: 4398654 | 12332 | 3: 4566642 | 12335 | 3: 4593309 | 12338 | 3: 4692667 |
| 12344 | 3: 4732630 | 12345 | 3: 4732659 | 12354 | 3: 4874925 | 12355 | 3: 4874954 | 12373 | 3: 5268053 |
| 12377 | 3: 5309746 | 12383 | 3: 5436199 | 12384 | 3: 5462355 | 12408 | 3: 5998482 | 12422 | 3: 6207469 |
| 12424 | 3: 6282733 | 12425 | 3: 6293053 | 12432 | 3: 6517775 | 12443 | 3: 6741890 | 12444 | 3: 6771925 |
| 12445 | 3: 6771928 | 12446 | 3: 6771969 | 12447 | 3: 6772038 | 12469 | 3: 7060982 | 12470 | 3: 7106174 |
| 12478 | 3: 7235310 | 12479 | 3: 7238069 | 12481 | 3: 7255164 | 12483 | 3: 7357130 | 12486 | 3: 7383219 |
| 12492 | 3: 7455499 | 12506 | 3: 7653471 | 12511 | 3: 7783911 | 12512 | 3: 7822809 | 12514 | 3: 7838838 |
| 12522 | 3: 7931685 | 12523 | 3: 7931828 | 12524 | 3: 7931887 | 12528 | 3: 7961056 | 12537 | 3: 8147516 |
| 12538 | 3: 8163086 | 12539 | 3: 8174824 | 12540 | 3: 8183497 | 12541 | 3: 8184872 | 12542 | 3: 8186958 |
| 12543 | 3: 8193625 | 12544 | 3: 8194122 | 12545 | 3: 8208066 | 12551 | 3: 8277038 | 12552 | 3: 8300299 |
| 12555 | 3: 8337519 | 12570 | 3: 8661008 | 12584 | 3: 9427220 | 12587 | 3: 9485193 | 12588 | 3: 9485906 |
| 12590 | 3: 9513246 | 12594 | 3: 9564986 | 12595 | 3: 9591739 | 12598 | 3: 9636755 | 12605 | 3: 9764080 |
| 12606 | 3: 9832615 | 12608 | 3: 9847485 | 12609 | 3: 9858051 | 12612 | 3: 9863664 | 12622 | 3: 10118782 |
| 12623 | 3: 10118907 | 12624 | 3: 10119257 | 12625 | 3: 10119319 | 12626 | 3: 10122375 | 12627 | 3: 10122970 |
| 12628 | 3: 10123769 | 12629 | 3: 10128298 | 12630 | 3: 10132847 | 12631 | 3: 10133516 | 12632 | 3: 10134377 |
| 12633 | 3: 10136175 | 12634 | 3: 10137707 | 12635 | 3: 10140467 | 12636 | 3: 10141169 | 12637 | 3: 10156326 |
| 12638 | 3: 10159182 | 12639 | 3: 10177781 | 12641 | 3: 10178026 | 12642 | 3: 10178076 | 12643 | 3: 10178165 |
| 12644 | 3: 10178365 | 12645 | 3: 10178537 | 12652 | 3: 10343580 | 12656 | 3: 10373626 | 12662 | 3: 10458829 |
| 12663 | 3: 10458901 | 12664 | 3: 10458904 | 12665 | 3: 10458919 | 12666 | 3: 10458930 | 12667 | 3: 10458938 |
| 12668 | 3: 10459027 | 12669 | 3: 10459091 | 12670 | 3: 10459139 | 12671 | 3: 10459202 | 12672 | 3: 10459244 |
| 12673 | 3: 10459247 | 12674 | 3: 10459248 | 12675 | 3: 10459299 | 12676 | 3: 10459407 | 12677 | 3: 10459424 |
| 12679 | 3: 10497816 | 12680 | 3: 10497887 | 12681 | 3: 10497945 | 12682 | 3: 10497948 | 12683 | 3: 10497963 |
| 12684 | 3: 10498011 | 12686 | 3: 10519036 | 12688 | 3: 10519145 | 12689 | 3: 10526199 | 12690 | 3: 10526562 |
| 12691 | 3: 10526604 | 12692 | 3: 10527562 | 12693 | 3: 10527602 | 12694 | 3: 10527675 | 12700 | 3: 10725165 |
| 12703 | 3: 10854110 | 12704 | 3: 10854114 | 12705 | 3: 10854133 | 12706 | 3: 10854173 | 12707 | 3: 10854726 |
| 12708 | 3: 10854773 | 12710 | 3: 10868768 | 12738 | 3: 11492432 | 12739 | 3: 11524924 | 12740 | 3: 11525352 |
| 12741 | 3: 11525876 | 12744 | 3: 11607381 | 12746 | 3: 11634350 | 12747 | 3: 11634398 | 12748 | 3: 11635619 |
| 12762 | 3: 12154189 | 12786 | 3: 12876946 | 12792 | 3: 13018051 | 12799 | 3: 13164663 | 12815 | 3: 13606552 |
| 12818 | 3: 13623598 | 12819 | 3: 13623632 | 12820 | 3: 13624757 | 12829 | 3: 13851188 | 12839 | 3: 13973650 |
| 12841 | 3: 13997525 | 12844 | 3: 14032792 | 12848 | 3: 14146757 | 12849 | 3: 14158851 | 12850 | 3: 14172051 |
| 12851 | 3: 14184603 | 12852 | 3: 14185315 | 12853 | 3: 14185422 | 12854 | 3: 14188705 | 12857 | 3: 14233203 |
| 12858 | 3: 14239901 | 12859 | 3: 14259325 | 12862 | 3: 14312950 | 12867 | 3: 14498473 | 12868 | 3: 14500940 |
| 12870 | 3: 14546904 | 12874 | 3: 14640547 | 12877 | 3: 14754130 | 12879 | 3: 14774051 | 12883 | 3: 14797864 |
| 12888 | 3: 14853239 | 12891 | 3: 14983274 | 12895 | 3: 15110207 | 12897 | 3: 15181630 | 12899 | 3: 15254497 |
| 12904 | 3: 15408885 | 12905 | 3: 15410634 | 12906 | 3: 15410653 | 12907 | 3: 15410660 | 12911 | 3: 15464742 |
| 12915 | 3: 15536943 | 12917 | 3: 15587630 | 12921 | 3: 15632301 | 12923 | 3: 15659384 | 12929 | 3: 15702216 |
| 12932 | 3: 15793894 | 12933 | 3: 15794524 | 12945 | 3: 15926790 | 12950 | 3: 16002303 | 12955 | 3: 16101556 |
| 12956 | 3: 16101690 | 12957 | 3: 16153064 | 12961 | 3: 16228126 | 12964 | 3: 16347204 | 12978 | 3: 16700799 |
| 12979 | 3: 16719574 | 12980 | 3: 16719945 | 12981 | 3: 16720763 | 12982 | 3: 16720823 | 12983 | 3: 16720842 |
| 12984 | 3: 16721218 | 12985 | 3: 16723559 | 12986 | 3: 16725242 | 13003 | 3: 17249309 | 13035 | 3: 18290761 |
| 13058 | 3: 18945520 | 13061 | 3: 19021728 | 13063 | 3: 19054577 | 13070 | 3: 19211257 | 13071 | 3: 19217706 |
| 13072 | 3: 19217713 | 13073 | 3: 19218362 | 13074 | 3: 19225387 | 13075 | 3: 19225406 | 13076 | 3: 19225421 |
| 13077 | 3: 19225424 | 13078 | 3: 19225426 | 13079 | 3: 19225509 | 13082 | 3: 19277608 | 13089 | 3: 19513415 |
| 13094 | 3: 19685974 | 13097 | 3: 19711385 | 13099 | 3: 19775942 | 13101 | 3: 19816181 | 13109 | 3: 20143190 |
| 13110 | 3: 20145668 | 13112 | 3: 20160583 | 13116 | 3: 20338589 | 13123 | 3: 20549806 | 13125 | 3: 20564672 |
| 13130 | 3: 20805323 | 13132 | 3: 20877656 | 13142 | 3: 21424575 | 13168 | 3: 22009877 | 13173 | 3: 22059782 |
| 13178 | 3: 22108825 | 13201 | 3: 22592195 | 13206 | 3: 22780511 | 13207 | 3: 22808543 | 13209 | 3: 22841288 |
| 13211 | 3: 22869028 | 13214 | 3: 22938448 | 13215 | 3: 22963089 | 13219 | 3: 23051090 | 13220 | 3: 23087313 |
| 13225 | 3: 23186080 | 13231 | 3: 23299798 | 13233 | 3: 23367141 | 13235 | 3: 23401470 | 13242 | 3: 23543571 |
| 13259 | 3: 24023430 | 13260 | 3: 24023439 | 13265 | 3: 24145693 | 13266 | 3: 24151362 | 13269 | 3: 24196352 |
| 13270 | 3: 24213490 | 13272 | 3: 24238856 | 13281 | 3: 24394654 | 13283 | 3: 24424423 | 13301 | 3: 24950077 |
| 13308 | 3: 25150752 | 13310 | 3: 25164615 | 13311 | 3: 25170221 | 13312 | 3: 25173303 | 13331 | 3: 25597219 |
| 13332 | 3: 25600844 | 13344 | 3: 26002177 | 13346 | 3: 26027856 | 13347 | 3: 26036214 | 13348 | 3: 26044741 |
| 13349 | 3: 26046291 | 13359 | 3: 26212407 | 13360 | 3: 26212494 | 13361 | 3: 26212691 | 13363 | 3: 26256361 |
| 13371 | 3: 26380532 | 13372 | 3: 26391670 | 13377 | 3: 26439980 | 13378 | 3: 26469071 | 13379 | 3: 26473706 |
| 13391 | 3: 26893372 | 13395 | 3: 26962431 | 13397 | 3: 26979935 | 13399 | 3: 27052772 | 13400 | 3: 27057546 |
| 13401 | 3: 27081349 | 13409 | 3: 27180648 | 13413 | 3: 27264556 | 13414 | 3: 27294196 | 13417 | 3: 27380069 |
| 13418 | 3: 27382883 | 13419 | 3: 27384412 | 13423 | 3: 27481652 | 13452 | 3: 27642990 | 13454 | 3: 27663059 |
| 13458 | 3: 27753499 | 13459 | 3: 27756989 | 13463 | 3: 27871144 | 13465 | 3: 27916123 | 13468 | 3: 27942850 |
| 13469 | 3: 27942868 | 13472 | 3: 27986412 | 13473 | 3: 27990865 | 13476 | 3: 28061404 | 13495 | 3: 28713706 |
| 13509 | 3: 29019861 | 13537 | 3: 29488699 | 13545 | 3: 29585223 | 13546 | 3: 29592827 | 13547 | 3: 29597761 |
| 13548 | 3: 29601864 | 13550 | 3: 29625965 | 13555 | 3: 29681833 | 13558 | 3: 29705190 | 13559 | 3: 29705342 |
| 13560 | 3: 29705512 | 13561 | 3: 29706025 | 13562 | 3: 29706295 | 13563 | 3: 29706336 | 13568 | 3: 29857727 |
| 13570 | 3: 29915442 | 13571 | 3: 29944912 | 13573 | 3: 29954086 | 13575 | 3: 29989198 | 13588 | 3: 30363529 |
| 13607 | 3: 30832758 | 13613 | 3: 30890540 | 13619 | 3: 31013256 | 13622 | 3: 31065769 | 13627 | 3: 31222103 |
| 13630 | 3: 31343441 | 13645 | 3: 31640573 | 13646 | 3: 31689225 | 13652 | 3: 31856497 | 13654 | 3: 31864015 |
| 13655 | 3: 31867053 | 13657 | 3: 31896363 | 13664 | 3: 32046129 | 13665 | 3: 32047685 | 13666 | 3: 32079966 |
| 13668 | 3: 32103555 | 13671 | 3: 32156925 | 13673 | 3: 32164330 | 13676 | 3: 32278723 | 13681 | 3: 32512486 |
| 13699 | 3: 32780141 | 13738 | 3: 32985901 | 13743 | 3: 33003020 | 13762 | 3: 33254753 | 13780 | 3: 33545907 |
| 13781 | 3: 33549574 | 13784 | 3: 33581206 | 13786 | 3: 33623020 | 13787 | 3: 33626853 | 13788 | 3: 33628444 |
| 13789 | 3: 33635163 | 13790 | 3: 33641481 | 13791 | 3: 33644276 | 13792 | 3: 33647252 | 13793 | 3: 33648187 |
| 13794 | 3: 33652206 | 13795 | 3: 33655047 | 13796 | 3: 33667890 | 13797 | 3: 33668793 | 13798 | 3: 33673078 |
| 13799 | 3: 33679729 | 13800 | 3: 33680460 | 13806 | 3: 33800750 | 13810 | 3: 33917904 | 13812 | 3: 33969180 |
| 13814 | 3: 33975547 | 13819 | 3: 34094670 | 13828 | 3: 34304343 | 13832 | 3: 34398092 | 13839 | 3: 34520818 |
| 13840 | 3: 34541029 | 13841 | 3: 34544910 | 13844 | 3: 34626494 | 13845 | 3: 34639336 | 13846 | 3: 34639367 |
| 13847 | 3: 34639414 | 13848 | 3: 34643371 | 13849 | 3: 34644168 | 13850 | 3: 34644227 | 13851 | 3: 34644230 |
| 13852 | 3: 34644292 | 13853 | 3: 34644311 | 13854 | 3: 34646033 | 13855 | 3: 34648675 | 13856 | 3: 34648858 |
| 13857 | 3: 34654205 | 13858 | 3: 34702016 | 13862 | 3: 34749792 | 13865 | 3: 34794868 | 13871 | 3: 34932689 |
| 13872 | 3: 34999359 | 13880 | 3: 35139460 | 13881 | 3: 35139484 | 13886 | 3: 35172154 | 13889 | 3: 35229235 |
| 13892 | 3: 35308462 | 13902 | 3: 35598523 | 13905 | 3: 35702543 | 13906 | 3: 35714205 | 13910 | 3: 35853066 |
| 13914 | 3: 35918054 | 13919 | 3: 36000811 | 13923 | 3: 36106918 | 13924 | 3: 36139522 | 13930 | 3: 36276403 |
| 13937 | 3: 36323043 | 13940 | 3: 36402532 | 13941 | 3: 36414305 | 13950 | 3: 36709252 | 13951 | 3: 36709316 |
| 13958 | 3: 36906114 | 13985 | 3: 37656931 | 13986 | 3: 37657062 | 13991 | 3: 37904335 | 14029 | 3: 39458322 |
| 14043 | 3: 39836580 | 14050 | 3: 40045551 | 14052 | 3: 40104674 | 14057 | 3: 40188225 | 14075 | 3: 40698452 |
| 14081 | 3: 41015843 | 14084 | 3: 41043374 | 14088 | 3: 41141556 | 14093 | 3: 41367055 | 14097 | 3: 41491123 |
| 14100 | 3: 41527680 | 14101 | 3: 41534229 | 14103 | 3: 41589506 | 14104 | 3: 41589571 | 14107 | 3: 41614043 |
| 14113 | 3: 41934611 | 14114 | 3: 41939570 | 14115 | 3: 41940253 | 14117 | 3: 41944095 | 14118 | 3: 41945371 |
| 14119 | 3: 41946261 | 14124 | 3: 42082768 | 14136 | 3: 42496233 | 14137 | 3: 42502863 | 14140 | 3: 42614221 |
| 14141 | 3: 42635738 | 14143 | 3: 42650352 | 14144 | 3: 42659935 | 14148 | 3: 42725238 | 14152 | 3: 42769882 |
| 14155 | 3: 42840302 | 14156 | 3: 42872218 | 14159 | 3: 42900902 | 14160 | 3: 42978663 | 14165 | 3: 43002941 |
| 14167 | 3: 43038774 | 14175 | 3: 43325595 | 14176 | 3: 43325725 | 14177 | 3: 43334230 | 14182 | 3: 43389979 |
| 14183 | 3: 43397421 | 14185 | 3: 43426451 | 14193 | 3: 43680161 | 14194 | 3: 43696024 | 14195 | 3: 43698262 |
| 14196 | 3: 43698948 | 14203 | 3: 43873693 | 14206 | 3: 44017413 | 14215 | 3: 44305690 | 14226 | 3: 44620511 |
| 14235 | 3: 44902917 | 14238 | 3: 44948682 | 14260 | 3: 45663768 | 14298 | 3: 46955918 | 14299 | 3: 46978051 |
| 14309 | 3: 47252028 | 14315 | 3: 47485150 | 14333 | 3: 47811507 | 14352 | 3: 48155117 | 14353 | 3: 48159568 |
| 14363 | 3: 48456906 | 14367 | 3: 48577665 | 14372 | 3: 48681437 | 14374 | 3: 48741921 | 14385 | 3: 48923622 |
| 14390 | 3: 48954466 | 14403 | 3: 49283986 | 14413 | 3: 49441533 | 14414 | 3: 49442661 | 14417 | 3: 49485613 |
| 14418 | 3: 49491032 | 14419 | 3: 49499289 | 14474 | 3: 49732105 | 14477 | 3: 49835535 | 14478 | 3: 49879024 |
| 14479 | 3: 49897610 | 14501 | 3: 50633952 | 14516 | 3: 51122225 | 14527 | 3: 51610699 | 14530 | 3: 51645901 |
| 14532 | 3: 51683713 | 14534 | 3: 51740729 | 14535 | 3: 51755409 | 14603 | 3: 53343252 | 14616 | 3: 53703853 |
| 14626 | 3: 54144822 | 14628 | 3: 54149746 | 14629 | 3: 54167611 | 14630 | 3: 54178592 | 14631 | 3: 54185471 |
| 14632 | 3: 54186364 | 14633 | 3: 54188660 | 14634 | 3: 54201062 | 14638 | 3: 54367531 | 14649 | 3: 55014493 |
| 14650 | 3: 55022327 | 14708 | 3: 56670745 | 14714 | 3: 56924360 | 14716 | 3: 56934727 | 14717 | 3: 56936672 |
| 14719 | 3: 56953053 | 14721 | 3: 56999586 | 14723 | 3: 57032150 | 14727 | 3: 57245572 | 14745 | 3: 57799143 |
| 14749 | 3: 57886444 | 14757 | 3: 58161126 | 14767 | 3: 58455621 | 14770 | 3: 58489145 | 14772 | 3: 58507559 |
| 14776 | 3: 58545248 | 14787 | 3: 58913251 | 14791 | 3: 59017336 | 14795 | 3: 59162716 | 14796 | 3: 59207859 |
| 14816 | 3: 59758400 | 14821 | 3: 60026204 | 14826 | 3: 60180066 | 14831 | 3: 60261618 | 14849 | 3: 60625549 |
| 14858 | 3: 60737754 | 14867 | 3: 60914592 | 14869 | 3: 60962012 | 14888 | 3: 61496668 | 14893 | 3: 61744158 |
| 14896 | 3: 61858795 | 14904 | 3: 62095195 | 14910 | 3: 62278399 | 14914 | 3: 62359529 | 14933 | 3: 62854439 |
| 14945 | 3: 63290027 | 14972 | 3: 63993039 | 14976 | 3: 64069515 | 14991 | 3: 64651171 | 14995 | 3: 64790813 |
| 15006 | 3: 65095548 | 15008 | 3: 65157057 | 15028 | 3: 65566921 | 15032 | 3: 65663032 | 15057 | 3: 66345116 |
| 15058 | 3: 66351139 | 15061 | 3: 66398657 | 15066 | 3: 66502134 | 15070 | 3: 66529229 | 15078 | 3: 66637168 |
| 15083 | 3: 66838529 | 15089 | 3: 66948480 | 15091 | 3: 66974396 | 15092 | 3: 66999361 | 15093 | 3: 67031006 |
| 15095 | 3: 67168089 | 15102 | 3: 67318712 | 15109 | 3: 67473906 | 15116 | 3: 67670465 | 15134 | 3: 68047509 |
| 15140 | 3: 68228132 | 15141 | 3: 68228231 | 15142 | 3: 68276086 | 15143 | 3: 68302197 | 15145 | 3: 68316834 |
| 15149 | 3: 68422911 | 15151 | 3: 68443638 | 15156 | 3: 68551519 | 15158 | 3: 68594137 | 15165 | 3: 68727455 |
| 15166 | 3: 68788461 | 15173 | 3: 68973246 | 15174 | 3: 69004319 | 15177 | 3: 69066161 | 15184 | 3: 69392637 |
| 15226 | 3: 70427777 | 15229 | 3: 70602082 | 15230 | 3: 70652398 | 15255 | 3: 71704029 | 15272 | 3: 72307570 |
| 15273 | 3: 72307597 | 15294 | 3: 73250579 | 15305 | 3: 73571608 | 15309 | 3: 73698701 | 15310 | 3: 73698721 |
| 15316 | 3: 73904881 | 15317 | 3: 73909382 | 15324 | 3: 74202751 | 15327 | 3: 74264677 | 15345 | 3: 74829928 |
| 15349 | 3: 74963727 | 15355 | 3: 75071726 | 15364 | 3: 75370499 | 15370 | 3: 75506540 | 15374 | 3: 75649317 |
| 15375 | 3: 75649320 | 15376 | 3: 75649336 | 15377 | 3: 75649383 | 15398 | 3: 76298830 | 15406 | 3: 76468869 |
| 15415 | 3: 76956223 | 15417 | 3: 77030221 | 15423 | 3: 77162998 | 15425 | 3: 77178882 | 15434 | 3: 77402844 |
| 15435 | 3: 77409935 | 15438 | 3: 77489908 | 15441 | 3: 77571521 | 15449 | 3: 77850286 | 15459 | 3: 78097452 |
| 15465 | 3: 78290774 | 15468 | 3: 78420363 | 15473 | 3: 78568977 | 15474 | 3: 78569227 | 15475 | 3: 78569835 |
| 15478 | 3: 78642264 | 15479 | 3: 78662366 | 15481 | 3: 78785400 | 15489 | 3: 79003182 | 15491 | 3: 79007539 |
| 15497 | 3: 79179242 | 15510 | 3: 79464743 | 15523 | 3: 79956010 | 15525 | 3: 79973859 | 15531 | 3: 80163495 |
| 15537 | 3: 80491657 | 15540 | 3: 80549623 | 15548 | 3: 80733840 | 15556 | 3: 80952119 | 15557 | 3: 80954660 |
| 15558 | 3: 80954761 | 15560 | 3: 80999275 | 15566 | 3: 81103725 | 15578 | 3: 81314640 | 15580 | 3: 81343738 |
| 15582 | 3: 81436844 | 15584 | 3: 81464927 | 15597 | 3: 81749835 | 15613 | 3: 82402375 | 15616 | 3: 82454574 |
| 15620 | 3: 82503792 | 15655 | 3: 83636824 | 15658 | 3: 83652637 | 15673 | 3: 84036094 | 15680 | 3: 84195762 |
| 15694 | 3: 84534786 | 15695 | 3: 84550534 | 15697 | 3: 84560190 | 15726 | 3: 84721616 | 15747 | 3: 85077748 |
| 15751 | 3: 85284944 | 15755 | 3: 85429614 | 15756 | 3: 85429668 | 15757 | 3: 85429696 | 15775 | 3: 85848368 |
| 15783 | 3: 85978048 | 15791 | 3: 86156348 | 15797 | 3: 86290419 | 15800 | 3: 86312689 | 15801 | 3: 86312736 |
| 15805 | 3: 86366796 | 15811 | 3: 86584920 | 15815 | 3: 86652496 | 15827 | 3: 86872323 | 15840 | 3: 87228581 |
| 15843 | 3: 87295571 | 15847 | 3: 87349602 | 15848 | 3: 87379701 | 15929 | 3: 88997759 | 15930 | 3: 89044393 |
| 15932 | 3: 89092242 | 15936 | 3: 89255790 | 15957 | 3: 89639867 | 15963 | 3: 89717766 | 15964 | 3: 89722428 |
| 15966 | 3: 89773983 | 15968 | 3: 89792849 | 15971 | 3: 89858003 | 15972 | 3: 89858025 | 15973 | 3: 89861756 |
| 15974 | 3: 89888858 | 15979 | 3: 89947137 | 15983 | 3: 90125909 | 15987 | 3: 90218397 | 15996 | 3: 90458410 |
| 15998 | 3: 90490960 | 16012 | 3: 90774079 | 16022 | 3: 90916704 | 16028 | 3: 91085209 | 16032 | 3: 91177765 |
| 16038 | 3: 91342742 | 16039 | 3: 91349417 | 16047 | 3: 91577320 | 16050 | 3: 91662702 | 16056 | 3: 91779733 |
| 16057 | 3: 91821560 | 16058 | 3: 91850874 | 16061 | 3: 91944104 | 16064 | 3: 92012709 | 16066 | 3: 92056509 |
| 16072 | 3: 92226650 | 16083 | 3: 92532036 | 16092 | 3: 92747667 | 16093 | 3: 92757686 | 16096 | 3: 92813097 |
| 16103 | 3: 92955459 | 16105 | 3: 92979991 | 16112 | 3: 93070031 | 16115 | 3: 93079352 | 16117 | 3: 93087724 |
| 16123 | 3: 93219307 | 16124 | 3: 93219814 | 16125 | 3: 93220543 | 16126 | 3: 93226524 | 16127 | 3: 93235948 |
| 16128 | 3: 93314047 | 16130 | 3: 93345899 | 16143 | 3: 93782247 | 16144 | 3: 93782331 | 16145 | 3: 93782436 |
| 16159 | 3: 94060442 | 16165 | 3: 94226523 | 16178 | 3: 94716721 | 16180 | 3: 94726765 | 16183 | 3: 94823429 |
| 16184 | 3: 94866313 | 16186 | 3: 94911090 | 16188 | 3: 94976568 | 16190 | 3: 95036766 | 16230 | 3: 96202292 |
| 16238 | 3: 96579309 | 16242 | 3: 96636004 | 16247 | 3: 96785820 | 16248 | 3: 96830575 | 16254 | 3: 96975581 |
| 16260 | 3: 97123954 | 16269 | 3: 97365473 | 16274 | 3: 97457441 | 16275 | 3: 97473661 | 16278 | 3: 97526789 |
| 16290 | 3: 97708909 | 16301 | 3: 97930159 | 16305 | 3: 98116083 | 16309 | 3: 98295744 | 16313 | 3: 98349294 |
| 16317 | 3: 98403571 | 16321 | 3: 98561508 | 16335 | 3: 98923450 | 16337 | 3: 98978448 | 16338 | 3: 98978458 |
| 16348 | 3: 99342353 | 16350 | 3: 99349479 | 16351 | 3: 99356345 | 16352 | 3: 99360354 | 16353 | 3: 99377234 |
| 16358 | 3: 99519922 | 16368 | 3: 99751257 | 16369 | 3: 99765693 | 16370 | 3: 99771380 | 16371 | 3: 99805386 |
| 16372 | 3: 99811235 | 16373 | 3: 99811520 | 16374 | 3: 99818061 | 16375 | 3: 99831451 | 16376 | 3: 99840460 |
| 16377 | 3: 99840480 | 16378 | 3: 99884150 | 16381 | 3: 99928886 | 16383 | 3: 99953595 | 16387 | 3: 100006117 |
| 16388 | 3: 100006731 | 16391 | 3: 100135156 | 16393 | 3: 100148664 | 16403 | 3: 100386736 | 16405 | 3: 100413711 |
| 16406 | 3: 100417680 | 16407 | 3: 100429883 | 16410 | 3: 100503760 | 16411 | 3: 100519527 | 16412 | 3: 100527924 |
| 16413 | 3: 100532091 | 16415 | 3: 100578191 | 16417 | 3: 100604376 | 16418 | 3: 100616906 | 16420 | 3: 100649238 |
| 16421 | 3: 100655322 | 16422 | 3: 100660031 | 16425 | 3: 100706971 | 16426 | 3: 100710303 | 16435 | 3: 100839974 |
| 16440 | 3: 101045558 | 16449 | 3: 101184420 | 16456 | 3: 101301924 | 16457 | 3: 101328497 | 16462 | 3: 101381082 |
| 16496 | 3: 102252547 | 16500 | 3: 102323975 | 16502 | 3: 102346918 | 16504 | 3: 102360619 | 16505 | 3: 102430768 |
| 16506 | 3: 102432444 | 16515 | 3: 102664478 | 16516 | 3: 102684885 | 16517 | 3: 102684933 | 16518 | 3: 102684985 |
| 16520 | 3: 102736091 | 16521 | 3: 102753266 | 16524 | 3: 102804450 | 16529 | 3: 102845961 | 16530 | 3: 102846036 |
| 16531 | 3: 102853282 | 16536 | 3: 102881866 | 16555 | 3: 103364021 | 16560 | 3: 103438363 | 16564 | 3: 103569295 |
| 16566 | 3: 103617298 | 16567 | 3: 103655132 | 16569 | 3: 103692819 | 16571 | 3: 103732748 | 16573 | 3: 103756143 |
| 16578 | 3: 103836979 | 16581 | 3: 103923977 | 16583 | 3: 104007365 | 16584 | 3: 104022881 | 16590 | 3: 104089157 |
| 16598 | 3: 104333611 | 16610 | 3: 104577770 | 16612 | 3: 104588926 | 16614 | 3: 104626150 | 16616 | 3: 104664666 |
| 16618 | 3: 104700803 | 16621 | 3: 104722133 | 16622 | 3: 104733085 | 16624 | 3: 104818821 | 16629 | 3: 104879046 |
| 16641 | 3: 105195217 | 16642 | 3: 105195508 | 16644 | 3: 105197594 | 16645 | 3: 105207363 | 16648 | 3: 105223366 |
| 16649 | 3: 105224129 | 16650 | 3: 105224197 | 16656 | 3: 105344373 | 16657 | 3: 105346169 | 16658 | 3: 105386965 |
| 16659 | 3: 105397156 | 16660 | 3: 105399284 | 16661 | 3: 105401478 | 16664 | 3: 105416679 | 16665 | 3: 105419956 |
| 16666 | 3: 105420955 | 16668 | 3: 105522467 | 16669 | 3: 105528154 | 16670 | 3: 105530865 | 16671 | 3: 105550247 |
| 16672 | 3: 105553161 | 16673 | 3: 105557267 | 16675 | 3: 105587522 | 16676 | 3: 105587615 | 16677 | 3: 105588435 |
| 16678 | 3: 105589110 | 16679 | 3: 105589417 | 16680 | 3: 105589499 | 16681 | 3: 105589515 | 16682 | 3: 105589567 |
| 16683 | 3: 105589840 | 16686 | 3: 105615307 | 16687 | 3: 105623790 | 16688 | 3: 105633378 | 16689 | 3: 105633804 |
| 16691 | 3: 105644182 | 16692 | 3: 105654662 | 16693 | 3: 105674940 | 16696 | 3: 105745106 | 16701 | 3: 105803674 |
| 16702 | 3: 105805027 | 16705 | 3: 105897358 | 16706 | 3: 105897511 | 16707 | 3: 105897845 | 16708 | 3: 105897970 |
| 16709 | 3: 105908966 | 16710 | 3: 105909058 | 16712 | 3: 105935499 | 16713 | 3: 105956333 | 16717 | 3: 106020186 |
| 16724 | 3: 106135737 | 16731 | 3: 106244085 | 16735 | 3: 106337682 | 16745 | 3: 106561725 | 16746 | 3: 106561875 |
| 16747 | 3: 106585197 | 16748 | 3: 106591729 | 16754 | 3: 106655656 | 16755 | 3: 106668839 | 16765 | 3: 106845702 |
| 16771 | 3: 107103522 | 16783 | 3: 107354102 | 16784 | 3: 107375067 | 16785 | 3: 107375088 | 16786 | 3: 107375142 |
| 16789 | 3: 107445270 | 16791 | 3: 107477138 | 16800 | 3: 107604142 | 16807 | 3: 107789881 | 16817 | 3: 107937893 |
| 16819 | 3: 107940715 | 16830 | 3: 108203613 | 16831 | 3: 108224103 | 16833 | 3: 108305450 | 16835 | 3: 108345650 |
| 16850 | 3: 108755292 | 16851 | 3: 108756407 | 16852 | 3: 108760658 | 16853 | 3: 108761540 | 16854 | 3: 108763978 |
| 16855 | 3: 108764835 | 16856 | 3: 108773793 | 16857 | 3: 108777623 | 16858 | 3: 108778270 | 16860 | 3: 108788102 |
| 16861 | 3: 108817854 | 16862 | 3: 108824614 | 16864 | 3: 108842377 | 16885 | 3: 109426329 | 16888 | 3: 109430792 |
| 16889 | 3: 109430841 | 16899 | 3: 109606532 | 16900 | 3: 109656896 | 16916 | 3: 109969045 | 6920 | 3: 110020722 |
| 16921 | 3: 110026915 | 16923 | 3: 110036182 | 16927 | 3: 110153404 | 16946 | 3: 110780122 | 16947 | 3: 110781851 |
| 16948 | 3: 110783875 | 16949 | 3: 110784912 | 16950 | 3: 110786097 | 16954 | 3: 110849640 | 16978 | 3: 111418313 |
| 16986 | 3: 111526183 | 16989 | 3: 111580049 | 17003 | 3: 111889624 | 17019 | 3: 112192839 | 17025 | 3: 112323001 |
| 17026 | 3: 112345897 | 17032 | 3: 112397006 | 17046 | 3: 112761486 | 17047 | 3: 112761940 | 17050 | 3: 112794864 |
| 17058 | 3: 113036203 | 17062 | 3: 113127829 | 17063 | 3: 113131422 | 17064 | 3: 113146141 | 17072 | 3: 113331205 |
| 17073 | 3: 113331220 | 17074 | 3: 113337934 | 17075 | 3: 113341660 | 17078 | 3: 113407286 | 17081 | 3: 113453006 |
| 17083 | 3: 113518551 | 17084 | 3: 113518636 | 17085 | 3: 113524422 | 17089 | 3: 113563964 | 17090 | 3: 113563979 |
| 17091 | 3: 113563997 | 17092 | 3: 113564006 | 17093 | 3: 113564076 | 17094 | 3: 113564096 | 17095 | 3: 113564107 |
| 17096 | 3: 113585771 | 17097 | 3: 113585795 | 17098 | 3: 113585799 | 17100 | 3: 113586101 | 17101 | 3: 113586251 |
| 17102 | 3: 113590170 | 17120 | 3: 114115462 | 17126 | 3: 114165101 | 17137 | 3: 114384157 | 17141 | 3: 114446007 |
| 17144 | 3: 114496084 | 17146 | 3: 114508185 | 17149 | 3: 114543461 | 17150 | 3: 114548928 | 17152 | 3: 114594768 |
| 17156 | 3: 114618765 | 17160 | 3: 114704691 | 17164 | 3: 114748408 | 17175 | 3: 114802567 | 17185 | 3: 114881902 |
| 17190 | 3: 115000401 | 17193 | 3: 115052234 | 17199 | 3: 115107550 | 17200 | 3: 115120882 | 17208 | 3: 115363288 |
| 17210 | 3: 115374146 | 17213 | 3: 115397893 | 17222 | 3: 115484538 | 17225 | 3: 115543657 | 17227 | 3: 115544750 |
| 17231 | 3: 115602232 | 17232 | 3: 115602518 | 17237 | 3: 115733885 | 17238 | 3: 115814948 | 17243 | 3: 115936238 |
| 17246 | 3: 115967688 | 17262 | 3: 116221838 | 17264 | 3: 116272992 | 17270 | 3: 116347067 | 17282 | 3: 116562510 |
| 17291 | 3: 116772922 | 17299 | 3: 116861866 | 17310 | 3: 116928313 | 17314 | 3: 116981402 | 17315 | 3: 116981583 |
| 17316 | 3: 116981638 | 17320 | 3: 117000919 | 17323 | 3: 117031812 | 17324 | 3: 117049753 | 17326 | 3: 117070587 |
| 17330 | 3: 117123078 | 17331 | 3: 117126904 | 17334 | 3: 117149901 | 17337 | 3: 117209899 | 17338 | 3: 117209960 |
| 17341 | 3: 117278310 | 17342 | 3: 117283114 | 17344 | 3: 117317452 | 17345 | 3: 117325008 | 17353 | 3: 117578080 |
| 17360 | 3: 117649528 | 17361 | 3: 117649537 | 17362 | 3: 117662078 | 17365 | 3: 117673624 | 17366 | 3: 117696207 |
| 17370 | 3: 117709813 | 17394 | 3: 118128778 | 17401 | 3: 118223118 | 17403 | 3: 118288926 | 17404 | 3: 118289376 |
| 17405 | 3: 118289408 | 17406 | 3: 118289679 | 17411 | 3: 118387538 | 17420 | 3: 118548276 | 17426 | 3: 118615619 |
| 17436 | 3: 118755947 | 17440 | 3: 118806132 | 17449 | 3: 118883045 | 17457 | 3: 118964562 | 17461 | 3: 119094347 |
| 17467 | 3: 119427638 | 17472 | 3: 119648530 | 17475 | 3: 119673122 | 17476 | 3: 119709097 | 17481 | 3: 119787932 |
| 17486 | 3: 119868121 | 17499 | 3: 120268455 | 17502 | 3: 120306744 | 17503 | 3: 120306964 | 17510 | 3: 120576521 |
| 17515 | 3: 120695918 | 17522 | 3: 120790650 | 17553 | 4: 1446328 | 17648 | 4: 4173794 | 17649 | 4: 4184943 |
| 17663 | 4: 4533982 | 17666 | 4: 4582744 | 17674 | 4: 4681609 | 17675 | 4: 4681672 | 17679 | 4: 4733530 |
| 17686 | 4: 4818261 | 17691 | 4: 4871624 | 17692 | 4: 4931247 | 17693 | 4: 4943634 | 17701 | 4: 5077162 |
| 17703 | 4: 5134591 | 17708 | 4: 5202035 | 17714 | 4: 5319445 | 17715 | 4: 5319859 | 17721 | 4: 5377091 |
| 17722 | 4: 5395548 | 17724 | 4: 5422821 | 17725 | 4: 5437743 | 17726 | 4: 5441832 | 17756 | 4: 6207303 |
| 17787 | 4: 7009102 | 17788 | 4: 7009111 | 17792 | 4: 7073207 | 17793 | 4: 7106157 | 17799 | 4: 7247371 |
| 17808 | 4: 7354530 | 17814 | 4: 7445669 | 17820 | 4: 7632416 | 17821 | 4: 7658763 | 17822 | 4: 7658787 |
| 17823 | 4: 7658831 | 17824 | 4: 7658948 | 17825 | 4: 7658962 | 17827 | 4: 7697381 | 17835 | 4: 7933911 |
| 17836 | 4: 7945782 | 17837 | 4: 7996659 | 17840 | 4: 8046313 | 17844 | 4: 8179768 | 17847 | 4: 8246510 |
| 17850 | 4: 8272646 | 17853 | 4: 8420098 | 17854 | 4: 8435396 | 17856 | 4: 8445348 | 17859 | 4: 8506844 |
| 17883 | 4: 9151646 | 17891 | 4: 9271969 | 17893 | 4: 9323578 | 17896 | 4: 9385962 | 17898 | 4: 9407447 |
| 17900 | 4: 9444470 | 17901 | 4: 9474683 | 17902 | 4: 9477621 | 17904 | 4: 9485074 | 17905 | 4: 9486301 |
| 17906 | 4: 9488068 | 17907 | 4: 9490132 | 17908 | 4: 9490936 | 17909 | 4: 9492909 | 17920 | 4: 9817295 |
| 17949 | 4: 10163330 | 17953 | 4: 10231259 | 17963 | 4: 10387935 | 17973 | 4: 10679109 | 17996 | 4: 11190947 |
| 17997 | 4: 11191071 | 18000 | 4: 11265836 | 18001 | 4: 11269876 | 18002 | 4: 11271703 | 18007 | 4: 11332982 |
| 18008 | 4: 11339823 | 18009 | 4: 11344256 | 18010 | 4: 11344907 | 18029 | 4: 11936615 | 18050 | 4: 12576634 |
| 18052 | 4: 12673015 | 18061 | 4: 12802609 | 18063 | 4: 12826295 | 18066 | 4: 12860940 | 18072 | 4: 13010035 |
| 18091 | 4: 13579464 | 18092 | 4: 13579504 | 18093 | 4: 13579532 | 18098 | 4: 13707039 | 18102 | 4: 13801041 |
| 18106 | 4: 14002326 | 18110 | 4: 14035164 | 18111 | 4: 14061507 | 18112 | 4: 14079747 | 18148 | 4: 15337479 |
| 18153 | 4: 15469076 | 18154 | 4: 15470050 | 18160 | 4: 15586640 | 18182 | 4: 16245146 | 18200 | 4: 16761268 |
| 18210 | 4: 17040967 | 18221 | 4: 17237895 | 18235 | 4: 17572154 | 18236 | 4: 17572159 | 18237 | 4: 17572202 |
| 18238 | 4: 17572213 | 18256 | 4: 18084841 | 18272 | 4: 18585854 | 18277 | 4: 18655237 | 18279 | 4: 18662663 |
| 18280 | 4: 18669066 | 18282 | 4: 18685097 | 18284 | 4: 18697683 | 18286 | 4: 18705074 | 18287 | 4: 18707492 |
| 18293 | 4: 18889879 | 18325 | 4: 19569259 | 18363 | 4: 19999023 | 18368 | 4: 20111420 | 18373 | 4: 20255515 |
| 18379 | 4: 20329251 | 18380 | 4: 20329405 | 18397 | 4: 20796684 | 18398 | 4: 20802780 | 18399 | 4: 20804901 |
| 18419 | 4: 21160009 | 18420 | 4: 21160045 | 18422 | 4: 21189572 | 18424 | 4: 21207059 | 18425 | 4: 21213102 |
| 18460 | 4: 22190378 | 18463 | 4: 22352000 | 18469 | 4: 22526571 | 18488 | 4: 22913622 | 18498 | 4: 23160284 |
| 18510 | 4: 23595303 | 18521 | 4: 23872526 | 18523 | 4: 23891166 | 18526 | 4: 23927655 | 18527 | 4: 23946714 |
| 18536 | 4: 24333008 | 18549 | 4: 24650631 | 18554 | 4: 24810069 | 18557 | 4: 24885290 | 18565 | 4: 25097829 |
| 18566 | 4: 25109810 | 18567 | 4: 25127084 | 18583 | 4: 25647682 | 18587 | 4: 25742989 | 18590 | 4: 25907759 |
| 18591 | 4: 25907762 | 18594 | 4: 25924624 | 18609 | 4: 26257023 | 18621 | 4: 26648480 | 18627 | 4: 26786277 |
| 18641 | 4: 27097020 | 18642 | 4: 27127515 | 18654 | 4: 27476944 | 18662 | 4: 27629208 | 18666 | 4: 27698262 |
| 18681 | 4: 28186796 | 18682 | 4: 28217559 | 18686 | 4: 28338304 | 18690 | 4: 28410444 | 18691 | 4: 28434228 |
| 18703 | 4: 28719533 | 18704 | 4: 28738300 | 18712 | 4: 28975908 | 18714 | 4: 29080932 | 18716 | 4: 29142901 |
| 18745 | 4: 30297344 | 18752 | 4: 30430428 | 18753 | 4: 30430450 | 18754 | 4: 30430542 | 18755 | 4: 30432018 |
| 18757 | 4: 30501662 | 18784 | 4: 31141033 | 18790 | 4: 31306787 | 18795 | 4: 31471170 | 18797 | 4: 31535187 |
| 18801 | 4: 31680539 | 18809 | 4: 31817533 | 18810 | 4: 31854356 | 18814 | 4: 31949743 | 18854 | 4: 33211684 |
| 18858 | 4: 33325825 | 18869 | 4: 33588518 | 18873 | 4: 33639372 | 18874 | 4: 33652107 | 18876 | 4: 33739841 |
| 18878 | 4: 33761684 | 18880 | 4: 33789343 | 18887 | 4: 33926324 | 18894 | 4: 34109934 | 18913 | 4: 34771597 |
| 18921 | 4: 35053751 | 18923 | 4: 35162460 | 18934 | 4: 35468668 | 18964 | 4: 36420563 | 18970 | 4: 36597277 |
| 18971 | 4: 36657445 | 18981 | 4: 36944816 | 18996 | 4: 37260138 | 19008 | 4: 37691142 | 19021 | 4: 38080777 |
| 19023 | 4: 38088154 | 19027 | 4: 38196269 | 19029 | 4: 38253124 | 19034 | 4: 38381571 | 19048 | 4: 38785910 |
| 19058 | 4: 39096980 | 19077 | 4: 39839146 | 19079 | 4: 39869029 | 19096 | 4: 40085698 | 19097 | 4: 40100668 |
| 19111 | 4: 40502117 | 19112 | 4: 40545187 | 19120 | 4: 40660943 | 19122 | 4: 40678229 | 19128 | 4: 40850556 |
| 19131 | 4: 40925102 | 19136 | 4: 40978581 | 19137 | 4: 40990306 | 19138 | 4: 40990370 | 19139 | 4: 40990413 |
| 19140 | 4: 40994417 | 19143 | 4: 41012545 | 19144 | 4: 41012589 | 19145 | 4: 41012593 | 19155 | 4: 41242597 |
| 19171 | 4: 42001110 | 19176 | 4: 42079882 | 19179 | 4: 42269505 | 19186 | 4: 42478244 | 19198 | 4: 42862322 |
| 19217 | 4: 43233221 | 19218 | 4: 43233239 | 19219 | 4: 43233296 | 19220 | 4: 43233323 | 19221 | 4: 43233412 |
| 19223 | 4: 43330695 | 19234 | 4: 43684052 | 19242 | 4: 43934934 | 19244 | 4: 43942237 | 19248 | 4: 44063960 |
| 19253 | 4: 44178421 | 19256 | 4: 44256983 | 19257 | 4: 44268015 | 19260 | 4: 44305806 | 19268 | 4: 44547450 |
| 19282 | 4: 45052002 | 19298 | 4: 45602329 | 19311 | 4: 46002303 | 19315 | 4: 46099926 | 19317 | 4: 46129459 |
| 19329 | 4: 46600207 | 19340 | 4: 46830510 | 19345 | 4: 46921985 | 19346 | 4: 46925441 | 19347 | 4: 46925682 |
| 19348 | 4: 46943050 | 19349 | 4: 46943107 | 19350 | 4: 46943131 | 19363 | 4: 47258425 | 19379 | 4: 47479517 |
| 19383 | 4: 47511989 | 19386 | 4: 47569946 | 19397 | 4: 47904922 | 19399 | 4: 48028783 | 19403 | 4: 48241131 |
| 19420 | 4: 48780844 | 19426 | 4: 49017869 | 19428 | 4: 49022872 | 19430 | 4: 49035315 | 19431 | 4: 49035320 |
| 19432 | 4: 49035381 | 19433 | 4: 49035395 | 19434 | 4: 49035467 | 19435 | 4: 49037953 | 19437 | 4: 49045621 |
| 19438 | 4: 49045631 | 19439 | 4: 49045737 | 19440 | 4: 49045753 | 19441 | 4: 49045756 | 19442 | 4: 49045852 |
| 19444 | 4: 49061063 | 19445 | 4: 49062116 | 19447 | 4: 49107209 | 19452 | 4: 49181169 | 19456 | 4: 49269697 |
| 19457 | 4: 49284053 | 19467 | 4: 49523230 | 19478 | 4: 49842196 | 19482 | 4: 49964915 | 19486 | 4: 50057099 |
| 19489 | 4: 50248085 | 19490 | 4: 50345064 | 19496 | 4: 50538062 | 19515 | 4: 51250626 | 19517 | 4: 51303879 |
| 19526 | 4: 51543766 | 19532 | 4: 51713354 | 19581 | 4: 53246820 | 19594 | 4: 53516423 | 19599 | 4: 53675011 |
| 19614 | 4: 54062446 | 19622 | 4: 54498204 | 19634 | 4: 54973210 | 19638 | 4: 55064494 | 19643 | 4: 55358812 |
| 19646 | 4: 55397907 | 19658 | 4: 55841154 | 19659 | 4: 55864590 | 19660 | 4: 55873977 | 19663 | 4: 55914722 |
| 19665 | 4: 55965169 | 19671 | 4: 56042508 | 19674 | 4: 56091771 | 19683 | 4: 56311977 | 19689 | 4: 56597023 |
| 19691 | 4: 56614952 | 19696 | 4: 56795183 | 19706 | 4: 57053471 | 19711 | 4: 57121533 | 19713 | 4: 57264096 |
| 19722 | 4: 57443352 | 19726 | 4: 57549751 | 19727 | 4: 57552140 | 19730 | 4: 57682773 | 19735 | 4: 57810438 |
| 19764 | 4: 58952251 | 19771 | 4: 59151344 | 19778 | 4: 59234606 | 19796 | 4: 59761295 | 19798 | 4: 59797184 |
| 19799 | 4: 59797205 | 19817 | 4: 60407425 | 19823 | 4: 60610913 | 19829 | 4: 60822961 | 19835 | 4: 61004115 |
| 19843 | 4: 61150481 | 19844 | 4: 61176028 | 19845 | 4: 61188646 | 19868 | 4: 61850527 | 19876 | 4: 62043721 |
| 19886 | 4: 62197348 | 19901 | 4: 62758226 | 19902 | 4: 62762359 | 19903 | 4: 62769531 | 19910 | 4: 62994404 |
| 19911 | 4: 63001098 | 19919 | 4: 63185750 | 19922 | 4: 63289063 | 19926 | 4: 63398159 | 19932 | 4: 63595012 |
| 19945 | 4: 63872067 | 19946 | 4: 63887485 | 19948 | 4: 63898910 | 19949 | 4: 63913879 | 19957 | 4: 64206739 |
| 19966 | 4: 64550273 | 19968 | 4: 64596464 | 19969 | 4: 64598653 | 19970 | 4: 64633068 | 19997 | 4: 65579692 |
| 19998 | 4: 65585572 | 20000 | 4: 65586522 | 20004 | 4: 65662118 | 20005 | 4: 65663680 | 20006 | 4: 65663924 |
| 20011 | 4: 65757109 | 20016 | 4: 65871790 | 20018 | 4: 65887212 | 20020 | 4: 65935466 | 20026 | 4: 66031691 |
| 20030 | 4: 66126342 | 20032 | 4: 66167545 | 20040 | 4: 66351372 | 20044 | 4: 66424351 | 20058 | 4: 66948425 |
| 20064 | 4: 67093419 | 20079 | 4: 67478622 | 20089 | 4: 67825161 | 20093 | 4: 67886242 | 20098 | 4: 67941747 |
| 20103 | 4: 68103259 | 20106 | 4: 68130974 | 20112 | 4: 68281786 | 20118 | 4: 68525805 | 20138 | 4: 69316258 |
| 20146 | 4: 69461895 | 20150 | 4: 69590761 | 20153 | 4: 69624135 | 20162 | 4: 69834879 | 20168 | 4: 69953830 |
| 20177 | 4: 70190883 | 20179 | 4: 70206948 | 20181 | 4: 70239423 | 20184 | 4: 70315106 | 20197 | 4: 70629516 |
| 20200 | 4: 70650674 | 20207 | 4: 70942839 | 20208 | 4: 70955859 | 20210 | 4: 71014142 | 20212 | 4: 71044933 |
| 20241 | 4: 71963805 | 20242 | 4: 71995369 | 20247 | 4: 72082867 | 20250 | 4: 72155086 | 20265 | 4: 72831364 |
| 20266 | 4: 72831400 | 20278 | 4: 73141876 | 20280 | 4: 73230353 | 20281 | 4: 73230433 | 20282 | 4: 73230506 |
| 20287 | 4: 73356120 | 20295 | 4: 73677765 | 20320 | 4: 74355629 | 20326 | 4: 74574092 | 20327 | 4: 74575187 |
| 20328 | 4: 74575293 | 20332 | 4: 74690642 | 20336 | 4: 74750397 | 20337 | 4: 74775088 | 20339 | 4: 74788299 |
| 20347 | 4: 74921800 | 20348 | 4: 74931461 | 20349 | 4: 74937132 | 20351 | 4: 74962440 | 20355 | 4: 75035303 |
| 20360 | 4: 75176025 | 20375 | 4: 75585274 | 20376 | 4: 75596693 | 20395 | 4: 75964360 | 20410 | 4: 76300291 |
| 20411 | 4: 76303426 | 20427 | 4: 76654098 | 20428 | 4: 76665449 | 20431 | 4: 76826893 | 20434 | 4: 77005397 |
| 20438 | 4: 77038646 | 20439 | 4: 77114794 | 20440 | 4: 77131669 | 20441 | 4: 77141818 | 20443 | 4: 77158468 |
| 20446 | 4: 77222263 | 20447 | 4: 77232107 | 20448 | 4: 77235523 | 20449 | 4: 77272270 | 20453 | 4: 77462163 |
| 20459 | 4: 77550845 | 20460 | 4: 77550847 | 20461 | 4: 77550961 | 20463 | 4: 77552548 | 20464 | 4: 77552605 |
| 20465 | 4: 77552647 | 20466 | 4: 77562640 | 20467 | 4: 77564937 | 20468 | 4: 77565007 | 20469 | 4: 77565031 |
| 20470 | 4: 77612846 | 20474 | 4: 77703279 | 20484 | 4: 78046476 | 20493 | 4: 78275533 | 20500 | 4: 78501007 |
| 20516 | 4: 79081070 | 20532 | 4: 79524078 | 20534 | 4: 79581997 | 20535 | 4: 79582037 | 20570 | 4: 80210945 |
| 20571 | 4: 80211747 | 20575 | 4: 80312765 | 20577 | 4: 80342472 | 20578 | 4: 80347612 | 20603 | 4: 81058244 |
| 20606 | 4: 81115882 | 20608 | 4: 81125829 | 20612 | 4: 81203512 | 20613 | 4: 81222120 | 20615 | 4: 81291786 |
| 20624 | 4: 81541207 | 20646 | 4: 82094311 | 20656 | 4: 82418721 | 20664 | 4: 82649849 | 20665 | 4: 82649930 |
| 20668 | 4: 82720591 | 20669 | 4: 82720596 | 20670 | 4: 82720610 | 20671 | 4: 82720657 | 20672 | 4: 82720686 |
| 20673 | 4: 82720715 | 20679 | 4: 82824766 | 20696 | 4: 83387242 | 20697 | 4: 83420381 | 20699 | 4: 83506707 |
| 20700 | 4: 83540447 | 20701 | 4: 83605440 | 20725 | 4: 84374337 | 20748 | 4: 84998314 | 20767 | 4: 85529127 |
| 20769 | 4: 85585852 | 20770 | 4: 85589724 | 20771 | 4: 85609908 | 20783 | 4: 85887734 | 20784 | 4: 85902484 |
| 20791 | 4: 86035429 | 20794 | 4: 86107048 | 20805 | 4: 86429759 | 20807 | 4: 86434813 | 20808 | 4: 86453759 |
| 20812 | 4: 86588536 | 20827 | 4: 86869600 | 20831 | 4: 87015587 | 20832 | 4: 87025872 | 20833 | 4: 87026389 |
| 20847 | 4: 87544345 | 20861 | 4: 87862791 | 20869 | 4: 87966655 | 20870 | 4: 87988315 | 20871 | 4: 88003339 |
| 20874 | 4: 88190387 | 20876 | 4: 88214757 | 20877 | 4: 88230965 | 20878 | 4: 88267363 | 20883 | 4: 88482339 |
| 20916 | 4: 89540510 | 20919 | 4: 89600401 | 20920 | 4: 89600435 | 20921 | 4: 89600533 | 20925 | 4: 89717462 |
| 20926 | 4: 89720129 | 20997 | 4: 91292909 | 21000 | 4: 91358381 | 21011 | 4: 91589372 | 21017 | 4: 91792117 |
| 21021 | 4: 91837317 | 21032 | 4: 92071091 | 21034 | 4: 92150036 | 21036 | 4: 92281567 | 21038 | 4: 92292551 |
| 21039 | 4: 92298261 | 21040 | 4: 92299550 | 21041 | 4: 92307140 | 21042 | 4: 92315009 | 21044 | 4: 92351942 |
| 21049 | 4: 92444591 | 21058 | 4: 92540924 | 21062 | 4: 92634658 | 21065 | 4: 92695366 | 21072 | 4: 92893919 |
| 21083 | 4: 93143125 | 21086 | 4: 93270718 | 21088 | 4: 93325674 | 21091 | 4: 93338554 | 21092 | 4: 93343119 |
| 21093 | 4: 93349924 | 21094 | 4: 93350879 | 21095 | 4: 93352485 | 21096 | 4: 93361239 | 21097 | 4: 93361477 |
| 21098 | 4: 93362689 | 21099 | 4: 93368708 | 21100 | 4: 93373657 | 21101 | 4: 93376458 | 21102 | 4: 93388339 |
| 21104 | 4: 93445593 | 21107 | 4: 93470782 | 21114 | 4: 93581039 | 21121 | 4: 93756393 | 21125 | 4: 93956921 |
| 21138 | 4: 94448413 | 21147 | 4: 94698872 | 21148 | 4: 94699823 | 21151 | 4: 94815446 | 21152 | 4: 94833611 |
| 21156 | 4: 94917698 | 21157 | 4: 94918493 | 21169 | 4: 95254763 | 21170 | 4: 95254765 | 21171 | 4: 95254773 |
| 21172 | 4: 95254787 | 21175 | 4: 95294696 | 21178 | 4: 95382163 | 21196 | 4: 95826773 | 21198 | 4: 95871966 |
| 21203 | 4: 95901052 | 21207 | 4: 95975966 | 21221 | 4: 96011612 | 21233 | 4: 96120257 | 21234 | 4: 96121708 |
| 21235 | 4: 96145894 | 21236 | 4: 96192829 | 21237 | 4: 96196621 | 21241 | 4: 96307908 | 21249 | 4: 96528346 |
| 21250 | 4: 96528347 | 21285 | 4: 96829783 | 21359 | 4: 96940681 | 21365 | 4: 96997475 | 21371 | 4: 97113666 |
| 21416 | 4: 97172490 | 21421 | 4: 97266645 | 21444 | 4: 97789995 | 21450 | 4: 98087559 | 21454 | 4: 98210913 |
| 21455 | 4: 98212198 | 21457 | 4: 98226178 | 21458 | 4: 98226699 | 21459 | 4: 98239198 | 21463 | 4: 98381525 |
| 21464 | 4: 98406747 | 21470 | 4: 98545217 | 21472 | 4: 98604946 | 21473 | 4: 98617473 | 21474 | 4: 98640953 |
| 21477 | 4: 98765309 | 21482 | 4: 98852463 | 21483 | 4: 98862279 | 21492 | 4: 99462535 | 21498 | 4: 99648977 |
| 21499 | 4: 99650381 | 21500 | 4: 99652215 | 21509 | 4: 99864130 | 21511 | 4: 99877799 | 21513 | 4: 99884272 |
| 21515 | 4: 99907629 | 21518 | 4: 100010231 | 21521 | 4: 100048120 | 21522 | 4: 100048151 | 21533 | 4: 100235968 |
| 21558 | 4: 100865583 | 21560 | 4: 100891716 | 21564 | 4: 100958472 | 21569 | 4: 101138513 | 21580 | 4: 101273627 |
| 21581 | 4: 101278348 | 21582 | 4: 101283299 | 21583 | 4: 101286906 | 21584 | 4: 101304065 | 21594 | 4: 101562975 |
| 21595 | 4: 101570088 | 21597 | 4: 101615078 | 21599 | 4: 101627868 | 21604 | 4: 101697277 | 21606 | 4: 101718193 |
| 21607 | 4: 101720088 | 21608 | 4: 101796334 | 21609 | 4: 101817565 | 21612 | 4: 102044239 | 21613 | 4: 102053938 |
| 21623 | 4: 102294876 | 21630 | 4: 102465082 | 21631 | 4: 102515585 | 21632 | 4: 102515711 | 21633 | 4: 102515830 |
| 21635 | 4: 102567199 | 21636 | 4: 102567675 | 21637 | 4: 102571979 | 21638 | 4: 102577247 | 21641 | 4: 102679178 |
| 21650 | 4: 102994861 | 21653 | 4: 103066617 | 21656 | 4: 103118208 | 21672 | 4: 103464890 | 21673 | 4: 103466759 |
| 21678 | 4: 103589488 | 21691 | 4: 103984758 | 21705 | 4: 104406614 | 21713 | 4: 104554874 | 21714 | 4: 104639191 |
| 21719 | 4: 104710790 | 21721 | 4: 104731929 | 21726 | 4: 104879688 | 21730 | 4: 104955413 | 21739 | 4: 105212815 |
| 21742 | 4: 105331039 | 21743 | 4: 105332159 | 21747 | 4: 105369148 | 21753 | 4: 105480650 | 21754 | 4: 105480728 |
| 21755 | 4: 105482792 | 21766 | 4: 105750600 | 21767 | 4: 105757195 | 21768 | 4: 105785525 | 21773 | 4: 106025379 |
| 21775 | 4: 106025551 | 21780 | 4: 106081660 | 21781 | 4: 106085160 | 21782 | 4: 106088179 | 21783 | 4: 106088193 |
| 21784 | 4: 106088224 | 21785 | 4: 106088251 | 21786 | 4: 106088286 | 21792 | 4: 106177289 | 21796 | 4: 106243037 |
| 21797 | 4: 106246162 | 21798 | 4: 106257365 | 21807 | 4: 106459877 | 21808 | 4: 106461237 | 21809 | 4: 106461523 |
| 21810 | 4: 106462584 | 21811 | 4: 106463211 | 21812 | 4: 106526851 | 21817 | 4: 106674077 | 21820 | 4: 106723818 |
| 21821 | 4: 106724580 | 21822 | 4: 106738338 | 21823 | 4: 106768265 | 21827 | 4: 106823063 | 21833 | 4: 106915810 |
| 21835 | 4: 106985422 | 21837 | 4: 106991677 | 21838 | 4: 106991678 | 21839 | 4: 106991818 | 21840 | 4: 106991819 |
| 21841 | 4: 106993054 | 21842 | 4: 106993083 | 21843 | 4: 106993086 | 21844 | 4: 106993163 | 21845 | 4: 106993484 |
| 21846 | 4: 106993491 | 21847 | 4: 106993778 | 21848 | 4: 106993951 | 21849 | 4: 106993975 | 21850 | 4: 106994109 |
| 21851 | 4: 106994258 | 21852 | 4: 106997706 | 21853 | 4: 106998207 | 21857 | 4: 107044582 | 21858 | 4: 107070723 |
| 21859 | 4: 107075139 | 21867 | 4: 107202696 | 21868 | 4: 107202723 | 21869 | 4: 107202867 | 21870 | 4: 107202876 |
| 21873 | 4: 107293705 | 21874 | 4: 107293767 | 21875 | 4: 107293937 | 21876 | 4: 107293975 | 21877 | 4: 107293984 |
| 21878 | 4: 107294409 | 21879 | 4: 107294413 | 21884 | 4: 107599054 | 21891 | 4: 107760331 | 21896 | 4: 107893188 |
| 21918 | 4: 108666311 | 21919 | 4: 108666329 | 21936 | 4: 109206944 | 21996 | 4: 110555410 | 21998 | 4: 110627140 |
| 22003 | 4: 110751708 | 22004 | 4: 110751716 | 22005 | 4: 110759035 | 22006 | 4: 110759043 | 22007 | 4: 110759085 |
| 22008 | 4: 110759138 | 22009 | 4: 110759193 | 22012 | 4: 110870537 | 22017 | 4: 110963804 | 22018 | 4: 110997685 |
| 22021 | 4: 111094939 | 22027 | 4: 111132995 | 22030 | 4: 111174187 | 22036 | 4: 111290634 | 22041 | 4: 111393405 |
| 22042 | 4: 111393524 | 22060 | 4: 111696373 | 22072 | 4: 111836637 | 22075 | 4: 111906579 | 22076 | 4: 111910434 |
| 22077 | 4: 111911436 | 22079 | 4: 111972681 | 22085 | 4: 112108098 | 22087 | 4: 112136295 | 22090 | 4: 112153870 |
| 22091 | 4: 112155488 | 22093 | 4: 112172784 | 22097 | 4: 112238690 | 22100 | 4: 112269261 | 22110 | 4: 112424206 |
| 22112 | 4: 112448562 | 22113 | 4: 112452844 | 22117 | 4: 112516104 | 22122 | 4: 112599976 | 22124 | 4: 112652763 |
| 22125 | 4: 112660638 | 22126 | 4: 112663309 | 22127 | 4: 112664440 | 22128 | 4: 112665144 | 22129 | 4: 112671210 |
| 22130 | 4: 112675707 | 22131 | 4: 112676199 | 22132 | 4: 112677340 | 22133 | 4: 112681999 | 22136 | 4: 112716249 |
| 22138 | 4: 112739929 | 22139 | 4: 112740655 | 22140 | 4: 112741810 | 22141 | 4: 112748029 | 22142 | 4: 112749294 |
| 22143 | 4: 112754237 | 22144 | 4: 112755579 | 22145 | 4: 112769671 | 22146 | 4: 112770319 | 22147 | 4: 112785850 |
| 22153 | 4: 112842045 | 22154 | 4: 112844573 | 22155 | 4: 112855696 | 22157 | 4: 112873667 | 22158 | 4: 112873671 |
| 22159 | 4: 112873791 | 22160 | 4: 112874161 | 22162 | 4: 112920884 | 22163 | 4: 112920908 | 22164 | 4: 112958565 |
| 22178 | 4: 113478902 | 22184 | 4: 113579221 | 22185 | 4: 113579506 | 22186 | 4: 113584318 | 22187 | 4: 113584327 |
| 22188 | 4: 113584399 | 22189 | 4: 113584405 | 22190 | 4: 113587147 | 22199 | 4: 113617600 | 22200 | 4: 113617762 |
| 22201 | 4: 113622976 | 22220 | 4: 113685923 | 22221 | 4: 113693137 | 22222 | 4: 113706536 | 22230 | 4: 113862937 |
| 22232 | 4: 113865696 | 22233 | 4: 113865709 | 22234 | 4: 113865711 | 22235 | 4: 113889872 | 22236 | 4: 113924572 |
| 22239 | 4: 113963854 | 22242 | 4: 114103350 | 22247 | 4: 114278681 | 22261 | 4: 114688072 | 22262 | 4: 114690368 |
| 22263 | 4: 114690442 | 22264 | 4: 114691485 | 22265 | 4: 114692365 | 22270 | 4: 114751599 | 22271 | 4: 114758803 |
| 22273 | 4: 114770151 | 22275 | 4: 114805260 | 22276 | 4: 114812473 | 22277 | 4: 114812501 | 22278 | 4: 114812550 |
| 22279 | 4: 114812583 | 22289 | 4: 115031057 | 22290 | 4: 115033034 | 22291 | 4: 115043261 | 22292 | 4: 115066503 |
| 22293 | 4: 115067836 | 22294 | 4: 115071402 | 22295 | 4: 115072381 | 22296 | 4: 115074258 | 22297 | 4: 115075256 |
| 22298 | 4: 115077110 | 22299 | 4: 115077972 | 22300 | 4: 115079711 | 22301 | 4: 115105025 | 22304 | 4: 115127050 |
| 22305 | 4: 115127065 | 22309 | 4: 115153886 | 22310 | 4: 115153896 | 22311 | 4: 115163025 | 22326 | 4: 115413253 |
| 22327 | 4: 115413261 | 22328 | 4: 115413321 | 22329 | 4: 115413386 | 22330 | 4: 115413395 | 22359 | 4: 116192647 |
| 22360 | 4: 116192669 | 22361 | 4: 116192689 | 22362 | 4: 116192738 | 22363 | 4: 116192764 | 22379 | 4: 116445855 |
| 22402 | 4: 116915804 | 22408 | 4: 117000567 | 22409 | 4: 117000602 | 22410 | 4: 117000603 | 22420 | 4: 117210404 |
| 22427 | 4: 117486610 | 22428 | 4: 117505997 | 22448 | 4: 117829938 | 22465 | 4: 118353876 | 22466 | 4: 118358762 |
| 22472 | 4: 118419883 | 22476 | 4: 118517071 | 22485 | 4: 118646844 | 22490 | 4: 118756391 | 22494 | 4: 118829611 |
| 22496 | 4: 118829788 | 22504 | 4: 118939017 | 22523 | 4: 119457121 | 22535 | 4: 119689809 | 22553 | 5: 77074 |
| 22579 | 5: 827590 | 22580 | 5: 834238 | 22581 | 5: 882265 | 22582 | 5: 882349 | 22583 | 5: 882384 |
| 22584 | 5: 903638 | 22597 | 5: 1099972 | 22608 | 5: 1345105 | 22610 | 5: 1356051 | 22611 | 5: 1356065 |
| 22612 | 5: 1356090 | 22613 | 5: 1356140 | 22614 | 5: 1356177 | 22615 | 5: 1356191 | 22618 | 5: 1456630 |
| 22623 | 5: 1589478 | 22625 | 5: 1618964 | 22670 | 5: 2514974 | 22681 | 5: 2735966 | 22684 | 5: 2800666 |
| 22698 | 5: 3349859 | 22699 | 5: 3349891 | 22719 | 5: 4068348 | 22749 | 5: 4663849 | 22757 | 5: 4889807 |
| 22768 | 5: 5162198 | 22770 | 5: 5188536 | 22772 | 5: 5196871 | 22778 | 5: 5266489 | 22780 | 5: 5300800 |
| 22789 | 5: 5528412 | 22792 | 5: 5566952 | 22804 | 5: 5799927 | 22805 | 5: 5817089 | 22814 | 5: 6016548 |
| 22822 | 5: 6285112 | 22826 | 5: 6336054 | 22828 | 5: 6436778 | 22838 | 5: 6632102 | 22859 | 5: 7063231 |
| 22871 | 5: 7268739 | 22873 | 5: 7277360 | 22874 | 5: 7277411 | 22877 | 5: 7377438 | 22895 | 5: 7885365 |
| 22915 | 5: 8557986 | 22928 | 5: 8826886 | 22932 | 5: 8934422 | 22933 | 5: 8934467 | 22934 | 5: 8934474 |
| 22962 | 5: 9693605 | 22964 | 5: 9730547 | 22967 | 5: 9797519 | 22968 | 5: 9803049 | 22969 | 5: 9856996 |
| 22970 | 5: 9857138 | 22971 | 5: 9858180 | 22976 | 5: 9982908 | 22978 | 5: 10011555 | 22980 | 5: 10029875 |
| 22981 | 5: 10034496 | 22989 | 5: 10156211 | 22991 | 5: 10201512 | 22992 | 5: 10252022 | 22993 | 5: 10278854 |
| 23007 | 5: 10506388 | 23014 | 5: 10670341 | 23020 | 5: 10828061 | 23021 | 5: 10843507 | 23022 | 5: 10843627 |
| 23023 | 5: 10844849 | 23031 | 5: 11089678 | 23063 | 5: 12028271 | 23067 | 5: 12102588 | 23068 | 5: 12103176 |
| 23073 | 5: 12156021 | 23074 | 5: 12156678 | 23083 | 5: 12325031 | 23084 | 5: 12326106 | 23096 | 5: 12460715 |
| 23097 | 5: 12473390 | 23105 | 5: 12528945 | 23111 | 5: 12719804 | 23121 | 5: 13036017 | 23124 | 5: 13069760 |
| 23139 | 5: 13541155 | 23151 | 5: 14006938 | 23155 | 5: 14124180 | 23171 | 5: 14599931 | 23172 | 5: 14600013 |
| 23175 | 5: 14680469 | 23195 | 5: 15100766 | 23199 | 5: 15173290 | 23216 | 5: 15462968 | 23219 | 5: 15566954 |
| 23220 | 5: 15566955 | 23221 | 5: 15566965 | 23222 | 5: 15566966 | 23223 | 5: 15566972 | 23224 | 5: 15566977 |
| 23225 | 5: 15567052 | 23226 | 5: 15567070 | 23227 | 5: 15567096 | 23228 | 5: 15567111 | 23232 | 5: 15704626 |
| 23266 | 5: 16709205 | 23270 | 5: 16800463 | 23282 | 5: 17119922 | 23285 | 5: 17174787 | 23288 | 5: 17241349 |
| 23293 | 5: 17398558 | 23306 | 5: 17895598 | 23307 | 5: 17903579 | 23318 | 5: 18353350 | 23321 | 5: 18410507 |
| 23322 | 5: 18410599 | 23338 | 5: 18941613 | 23351 | 5: 19280880 | 23359 | 5: 19568730 | 23374 | 5: 19885896 |
| 23376 | 5: 19976107 | 23377 | 5: 19993960 | 23378 | 5: 20014240 | 23381 | 5: 20097603 | 23382 | 5: 20149313 |
| 23384 | 5: 20195300 | 23389 | 5: 20436830 | 23396 | 5: 20590589 | 23399 | 5: 20732321 | 23405 | 5: 20832737 |
| 23406 | 5: 20832885 | 23407 | 5: 20833047 | 23409 | 5: 20843567 | 23410 | 5: 20910258 | 23411 | 5: 20936220 |
| 23416 | 5: 21019650 | 23422 | 5: 21196220 | 23445 | 5: 21745511 | 23447 | 5: 21797550 | 23452 | 5: 21874789 |
| 23454 | 5: 21920920 | 23466 | 5: 22115062 | 23473 | 5: 22298578 | 23474 | 5: 22323880 | 23477 | 5: 22412419 |
| 23487 | 5: 22513497 | 23491 | 5: 22558133 | 23492 | 5: 22558154 | 23493 | 5: 22558155 | 23502 | 5: 22837489 |
| 23503 | 5: 22837951 | 23507 | 5: 22898017 | 23509 | 5: 22944167 | 23510 | 5: 22947768 | 23514 | 5: 23017065 |
| 23515 | 5: 23045588 | 23517 | 5: 23055987 | 23524 | 5: 23260695 | 23525 | 5: 23261205 | 23526 | 5: 23269298 |
| 23530 | 5: 23386932 | 23533 | 5: 23440852 | 23534 | 5: 23449942 | 23542 | 5: 23608812 | 23545 | 5: 23736650 |
| 23548 | 5: 23753028 | 23552 | 5: 23816911 | 23556 | 5: 23949886 | 23561 | 5: 23975472 | 23570 | 5: 24168834 |
| 23578 | 5: 24352659 | 23579 | 5: 24352754 | 23587 | 5: 24564540 | 23591 | 5: 24706081 | 23594 | 5: 24861511 |
| 23595 | 5: 24861917 | 23596 | 5: 24862040 | 23597 | 5: 24863117 | 23598 | 5: 24864988 | 23599 | 5: 24865092 |
| 23610 | 5: 25042104 | 23618 | 5: 25424142 | 23619 | 5: 25424207 | 23622 | 5: 25531432 | 23627 | 5: 25660326 |
| 23629 | 5: 25668094 | 23632 | 5: 25770487 | 23635 | 5: 25865044 | 23641 | 5: 25948717 | 23642 | 5: 25953449 |
| 23648 | 5: 26274617 | 23652 | 5: 26346656 | 23653 | 5: 26359865 | 23654 | 5: 26372843 | 23655 | 5: 26380803 |
| 23661 | 5: 26513500 | 23662 | 5: 26516803 | 23663 | 5: 26527639 | 23664 | 5: 26531218 | 23672 | 5: 26728680 |
| 23674 | 5: 26750096 | 23679 | 5: 26881116 | 23684 | 5: 27043324 | 23685 | 5: 27062257 | 23686 | 5: 27098658 |
| 23700 | 5: 27397526 | 23705 | 5: 27478824 | 23712 | 5: 27611131 | 23718 | 5: 27732993 | 23724 | 5: 27782046 |
| 23726 | 5: 27821648 | 23730 | 5: 27894859 | 23736 | 5: 28067367 | 23738 | 5: 28076547 | 23741 | 5: 28125774 |
| 23750 | 5: 28398456 | 23754 | 5: 28468312 | 23755 | 5: 28474788 | 23770 | 5: 28759211 | 23771 | 5: 28764621 |
| 23776 | 5: 28916451 | 23779 | 5: 28965670 | 23787 | 5: 29216390 | 23792 | 5: 29377131 | 23809 | 5: 29727121 |
| 23815 | 5: 29862958 | 23819 | 5: 29973188 | 23830 | 5: 30143566 | 23838 | 5: 30392463 | 23844 | 5: 30521361 |
| 23845 | 5: 30526943 | 23846 | 5: 30564929 | 23847 | 5: 30571021 | 23848 | 5: 30583097 | 23849 | 5: 30584287 |
| 23852 | 5: 30656853 | 23861 | 5: 30885641 | 23863 | 5: 30907588 | 23864 | 5: 30945722 | 23869 | 5: 31116777 |
| 23890 | 5: 31693587 | 23899 | 5: 32136613 | 23912 | 5: 32401173 | 23913 | 5: 32414358 | 23916 | 5: 32527988 |
| 23917 | 5: 32544707 | 23918 | 5: 32549914 | 23921 | 5: 32589543 | 23931 | 5: 32826960 | 23939 | 5: 33070073 |
| 23941 | 5: 33080199 | 23951 | 5: 33311326 | 23955 | 5: 33354800 | 23962 | 5: 33573362 | 23971 | 5: 33769971 |
| 23972 | 5: 33803288 | 23979 | 5: 33964757 | 23980 | 5: 33974899 | 24008 | 5: 34894503 | 24010 | 5: 34992883 |
| 24011 | 5: 34992887 | 24012 | 5: 34992888 | 24014 | 5: 35004904 | 24015 | 5: 35004932 | 24016 | 5: 35039187 |
| 24017 | 5: 35055317 | 24018 | 5: 35080229 | 24022 | 5: 35207804 | 24033 | 5: 35577195 | 24036 | 5: 35771527 |
| 24066 | 5: 36566052 | 24079 | 5: 37113142 | 24101 | 5: 37695072 | 24102 | 5: 37711059 | 24104 | 5: 37735001 |
| 24109 | 5: 37845630 | 24113 | 5: 37881472 | 24119 | 5: 37991676 | 24122 | 5: 38029636 | 24123 | 5: 38033300 |
| 24125 | 5: 38058027 | 24127 | 5: 38106278 | 24129 | 5: 38114225 | 24130 | 5: 38116179 | 24131 | 5: 38135398 |
| 24134 | 5: 38174231 | 24144 | 5: 38453061 | 24145 | 5: 38483708 | 24146 | 5: 38527379 | 24147 | 5: 38531595 |
| 24148 | 5: 38544831 | 24150 | 5: 38593359 | 24163 | 5: 39154317 | 24165 | 5: 39228037 | 24166 | 5: 39257315 |
| 24186 | 5: 39750764 | 24202 | 5: 40183299 | 24204 | 5: 40237324 | 24207 | 5: 40321836 | 24208 | 5: 40321842 |
| 24209 | 5: 40321905 | 24210 | 5: 40321940 | 24211 | 5: 40322458 | 24212 | 5: 40322491 | 24214 | 5: 40326831 |
| 24215 | 5: 40340341 | 24216 | 5: 40342525 | 24227 | 5: 40812862 | 24251 | 5: 41504706 | 24264 | 5: 41958537 |
| 24265 | 5: 41958597 | 24266 | 5: 41958622 | 24267 | 5: 41987705 | 24270 | 5: 42145772 | 24282 | 5: 42611303 |
| 24291 | 5: 42843742 | 24296 | 5: 42890909 | 24302 | 5: 43044298 | 24305 | 5: 43082557 | 24314 | 5: 43415290 |
| 24315 | 5: 43480306 | 24318 | 5: 43506882 | 24324 | 5: 43599378 | 24331 | 5: 43811761 | 24334 | 5: 43863604 |
| 24335 | 5: 43864090 | 24336 | 5: 43864115 | 24337 | 5: 43869544 | 24338 | 5: 43875060 | 24339 | 5: 43878846 |
| 24340 | 5: 43880997 | 24341 | 5: 43881099 | 24342 | 5: 43883078 | 24343 | 5: 43897037 | 24344 | 5: 43897095 |
| 24346 | 5: 43901763 | 24350 | 5: 44040614 | 24351 | 5: 44049829 | 24360 | 5: 44388064 | 24374 | 5: 44804564 |
| 24377 | 5: 44906731 | 24384 | 5: 45175795 | 24391 | 5: 45366260 | 24400 | 5: 45549099 | 24403 | 5: 45619287 |
| 24414 | 5: 45917363 | 24422 | 5: 46063715 | 24424 | 5: 46103767 | 24425 | 5: 46103871 | 24435 | 5: 46335844 |
| 24440 | 5: 46443847 | 24442 | 5: 46516496 | 24483 | 5: 47241615 | 24489 | 5: 47478077 | 24492 | 5: 47556051 |
| 24494 | 5: 47596785 | 24507 | 5: 48011805 | 24509 | 5: 48115271 | 24513 | 5: 48219521 | 24521 | 5: 48357494 |
| 24546 | 5: 49071305 | 24547 | 5: 49121969 | 24548 | 5: 49128930 | 24551 | 5: 49244193 | 24555 | 5: 49443860 |
| 24557 | 5: 49480432 | 24576 | 5: 50003215 | 24577 | 5: 50004728 | 24587 | 5: 50241038 | 24593 | 5: 50468141 |
| 24601 | 5: 50729869 | 24604 | 5: 50805474 | 24606 | 5: 50826119 | 24612 | 5: 50987421 | 24613 | 5: 50995228 |
| 24615 | 5: 51012775 | 24616 | 5: 51014179 | 24617 | 5: 51026281 | 24618 | 5: 51028777 | 24619 | 5: 51028823 |
| 24620 | 5: 51045771 | 24621 | 5: 51046494 | 24622 | 5: 51046653 | 24623 | 5: 51050849 | 24624 | 5: 51055590 |
| 24625 | 5: 51056409 | 24626 | 5: 51074757 | 24627 | 5: 51076137 | 24629 | 5: 51106212 | 24630 | 5: 51125457 |
| 24633 | 5: 51222282 | 24639 | 5: 51428229 | 24649 | 5: 51662795 | 24651 | 5: 51737595 | 24652 | 5: 51807509 |
| 24660 | 5: 52059878 | 24661 | 5: 52059908 | 24662 | 5: 52059930 | 24680 | 5: 52526329 | 24693 | 5: 53179786 |
| 24711 | 5: 53766718 | 24712 | 5: 53779538 | 24713 | 5: 53781567 | 24714 | 5: 53786730 | 24717 | 5: 53862521 |
| 24723 | 5: 54013034 | 24732 | 5: 54299758 | 24733 | 5: 54301166 | 24734 | 5: 54304681 | 24735 | 5: 54306088 |
| 24736 | 5: 54309409 | 24737 | 5: 54324570 | 24738 | 5: 54332158 | 24740 | 5: 54358159 | 24741 | 5: 54367083 |
| 24742 | 5: 54370187 | 24743 | 5: 54376482 | 24745 | 5: 54399560 | 24748 | 5: 54511599 | 24749 | 5: 54514698 |
| 24751 | 5: 54563791 | 24755 | 5: 54632888 | 24761 | 5: 54765009 | 24785 | 5: 55509920 | 24789 | 5: 55583411 |
| 24790 | 5: 55619423 | 24791 | 5: 55634685 | 24792 | 5: 55640765 | 24793 | 5: 55652450 | 24795 | 5: 55670462 |
| 24797 | 5: 55696567 | 24798 | 5: 55702652 | 24799 | 5: 55707061 | 24802 | 5: 55873933 | 24803 | 5: 55879578 |
| 24805 | 5: 55940985 | 24807 | 5: 55956661 | 24848 | 5: 56720568 | 24849 | 5: 56792015 | 24853 | 5: 56910201 |
| 24854 | 5: 56953231 | 24859 | 5: 57100134 | 24864 | 5: 57204918 | 24873 | 5: 57427084 | 24882 | 5: 57667264 |
| 24883 | 5: 57672067 | 24896 | 5: 58068683 | 24904 | 5: 58250121 | 24905 | 5: 58285910 | 24915 | 5: 58749685 |
| 24923 | 5: 59304558 | 24925 | 5: 59313596 | 24929 | 5: 59364960 | 24930 | 5: 59365133 | 24941 | 5: 59865035 |
| 24950 | 5: 60107106 | 24956 | 5: 60233686 | 24958 | 5: 60262488 | 24960 | 5: 60297442 | 24962 | 5: 60326833 |
| 24965 | 5: 60380312 | 24971 | 5: 60510721 | 24972 | 5: 60532648 | 24973 | 5: 60543258 | 24975 | 5: 60557011 |
| 24977 | 5: 60595067 | 24983 | 5: 60718806 | 24985 | 5: 60758142 | 24986 | 5: 60766663 | 24987 | 5: 60766858 |
| 24996 | 5: 61052215 | 25025 | 5: 62076373 | 25033 | 5: 62426367 | 25040 | 5: 62518142 | 25043 | 5: 62636512 |
| 25051 | 5: 62933839 | 25052 | 5: 62941170 | 25083 | 5: 63542114 | 25095 | 5: 63850062 | 25104 | 5: 64226221 |
| 25106 | 5: 64234434 | 25118 | 5: 64619628 | 25137 | 5: 65171005 | 25138 | 5: 65183093 | 25142 | 5: 65232220 |
| 25151 | 5: 65446380 | 25154 | 5: 65535107 | 25155 | 5: 65542737 | 25156 | 5: 65543322 | 25157 | 5: 65543372 |
| 25167 | 5: 65703423 | 25168 | 5: 65712111 | 25169 | 5: 65751283 | 25170 | 5: 65752854 | 25172 | 5: 65760426 |
| 25173 | 5: 65761371 | 25179 | 5: 66009583 | 25195 | 5: 66518756 | 25225 | 5: 67447645 | 25226 | 5: 67449152 |
| 25227 | 5: 67463576 | 25228 | 5: 67465088 | 25229 | 5: 67468026 | 25230 | 5: 67470940 | 25231 | 5: 67473722 |
| 25232 | 5: 67475068 | 25233 | 5: 67475725 | 25234 | 5: 67477065 | 25235 | 5: 67481131 | 25237 | 5: 67514487 |
| 25238 | 5: 67526075 | 25239 | 5: 67526864 | 25241 | 5: 67555054 | 25242 | 5: 67563065 | 25243 | 5: 67568345 |
| 25251 | 5: 67846005 | 25252 | 5: 67848289 | 25253 | 5: 67851400 | 25260 | 5: 68044942 | 25261 | 5: 68062320 |
| 25265 | 5: 68180283 | 25267 | 5: 68195298 | 25277 | 5: 68311976 | 25280 | 5: 68341993 | 25282 | 5: 68419217 |
| 25284 | 5: 68527931 | 25292 | 5: 68745462 | 25294 | 5: 68847086 | 25305 | 5: 68998098 | 25309 | 5: 69114492 |
| 25314 | 5: 69191792 | 25315 | 5: 69211669 | 25318 | 5: 69282729 | 25319 | 5: 69295982 | 25327 | 5: 69507467 |
| 25329 | 5: 69534170 | 25330 | 5: 69540325 | 25339 | 5: 69690113 | 25346 | 5: 69807021 | 25354 | 5: 70143301 |
| 25369 | 5: 70657641 | 25371 | 5: 70725053 | 25383 | 5: 70904784 | 25385 | 5: 70931624 | 25388 | 5: 71041333 |
| 25390 | 5: 71113115 | 25393 | 5: 71147255 | 25397 | 5: 71158676 | 25399 | 5: 71204940 | 25401 | 5: 71215860 |
| 25402 | 5: 71247169 | 25404 | 5: 71274477 | 25406 | 5: 71300632 | 25408 | 5: 71346087 | 25409 | 5: 71377241 |
| 25418 | 5: 71535392 | 25422 | 5: 71629712 | 25432 | 5: 71856285 | 25433 | 5: 71893342 | 25434 | 5: 71896515 |
| 25435 | 5: 71909707 | 25438 | 5: 71958333 | 25442 | 5: 72039709 | 25443 | 5: 72058094 | 25445 | 5: 72064330 |
| 25458 | 5: 72293857 | 25461 | 5: 72366005 | 25462 | 5: 72368884 | 25475 | 5: 72696496 | 25500 | 5: 73554593 |
| 25504 | 5: 73672740 | 25521 | 5: 73884415 | 25522 | 5: 73908825 | 25523 | 5: 73980075 | 25529 | 5: 74150522 |
| 25532 | 5: 74225942 | 25533 | 5: 74245072 | 25545 | 5: 74650187 | 25547 | 5: 74725927 | 25549 | 5: 74742101 |
| 25555 | 5: 74908285 | 25556 | 5: 74908338 | 25557 | 5: 74909826 | 25558 | 5: 74911263 | 25559 | 5: 74911341 |
| 25560 | 5: 74915786 | 25561 | 5: 74919175 | 25562 | 5: 74925227 | 25563 | 5: 74925818 | 25564 | 5: 74926028 |
| 25565 | 5: 74928887 | 25566 | 5: 74936574 | 25571 | 5: 75167453 | 25574 | 5: 75224410 | 25575 | 5: 75224450 |
| 25576 | 5: 75224458 | 25577 | 5: 75224463 | 25579 | 5: 75224524 | 25582 | 5: 75261093 | 25586 | 5: 75281166 |
| 25599 | 5: 75676623 | 25605 | 5: 75823353 | 25608 | 5: 75912892 | 25609 | 5: 75919398 | 25614 | 5: 75988734 |
| 25620 | 5: 76209127 | 25621 | 5: 76269564 | 25622 | 5: 76269603 | 25631 | 5: 76640050 | 25632 | 5: 76666195 |
| 25640 | 5: 76784217 | 25649 | 5: 77156529 | 25650 | 5: 77161120 | 25651 | 5: 77180731 | 25652 | 5: 77193464 |
| 25653 | 5: 77193475 | 25654 | 5: 77193500 | 25660 | 5: 77277820 | 25661 | 5: 77302753 | 25662 | 5: 77331990 |
| 25664 | 5: 77340074 | 25666 | 5: 77362757 | 25667 | 5: 77410429 | 25677 | 5: 77928157 | 25678 | 5: 77928784 |
| 25692 | 5: 78324681 | 25698 | 5: 78506395 | 25699 | 5: 78510349 | 25700 | 5: 78522919 | 25708 | 5: 78820352 |
| 25709 | 5: 78820587 | 25710 | 5: 78820588 | 25711 | 5: 78820618 | 25712 | 5: 78820726 | 25713 | 5: 78820795 |
| 25714 | 5: 78820814 | 25715 | 5: 78821054 | 25718 | 5: 78829751 | 25719 | 5: 78832107 | 25720 | 5: 78832141 |
| 25721 | 5: 78832271 | 25752 | 5: 79910151 | 25775 | 5: 80693563 | 25784 | 5: 80982109 | 25788 | 5: 81166934 |
| 25794 | 5: 81369156 | 25802 | 5: 81505086 | 25816 | 5: 82023846 | 25818 | 5: 82073695 | 25822 | 5: 82125171 |
| 25825 | 5: 82161343 | 25835 | 5: 82321735 | 25839 | 5: 82419134 | 25842 | 5: 82485421 | 25843 | 5: 82485429 |
| 25844 | 5: 82491588 | 25847 | 5: 82535021 | 25868 | 5: 82755673 | 25874 | 5: 83018943 | 25879 | 5: 83102062 |
| 25885 | 5: 83210516 | 25887 | 5: 83258916 | 25890 | 5: 83347445 | 25919 | 5: 83920522 | 25924 | 5: 84024864 |
| 26062 | 5: 84271561 | 26081 | 5: 84723773 | 26087 | 5: 84917552 | 26096 | 5: 85106613 | 26097 | 5: 85112695 |
| 26102 | 5: 85286730 | 26138 | 5: 86172964 | 26151 | 5: 86508471 | 26155 | 5: 86538135 | 26167 | 5: 86803983 |
| 26168 | 5: 86807041 | 26195 | 5: 87419158 | 26197 | 5: 87436653 | 26205 | 5: 87558801 | 26222 | 5: 87935293 |
| 26227 | 5: 88025590 | 26232 | 5: 88169363 | 26240 | 5: 88321354 | 26241 | 5: 88337164 | 26246 | 5: 88393821 |
| 26251 | 5: 88442816 | 26257 | 5: 88527567 | 26259 | 5: 88560515 | 26260 | 5: 88562589 | 26264 | 5: 88656117 |
| 26265 | 5: 88656655 | 26266 | 5: 88663005 | 26267 | 5: 88664137 | 26268 | 5: 88664970 | 26269 | 5: 88665685 |
| 26270 | 5: 88667764 | 26271 | 5: 88668736 | 26272 | 5: 88669208 | 26273 | 5: 88670166 | 26274 | 5: 88670949 |
| 26275 | 5: 88671581 | 26276 | 5: 88672838 | 26277 | 5: 88673518 | 26278 | 5: 88675186 | 26279 | 5: 88676434 |
| 26280 | 5: 88677664 | 26281 | 5: 88679714 | 26282 | 5: 88681101 | 26289 | 5: 88791076 | 26294 | 5: 88871429 |
| 26295 | 5: 88903163 | 26297 | 5: 88927675 | 26303 | 5: 89099225 | 26311 | 5: 89264046 | 26313 | 5: 89283625 |
| 26315 | 5: 89343653 | 26316 | 5: 89359278 | 26317 | 5: 89364312 | 26327 | 5: 89668965 | 26337 | 5: 89886383 |
| 26338 | 5: 89886385 | 26346 | 5: 90068114 | 26353 | 5: 90321950 | 26354 | 5: 90345643 | 26356 | 5: 90409538 |
| 26363 | 5: 90604091 | 26369 | 5: 90678336 | 26375 | 5: 90778808 | 26425 | 5: 92149419 | 26439 | 5: 92379543 |
| 26479 | 5: 92796508 | 26480 | 5: 92796509 | 26481 | 5: 92796641 | 26482 | 5: 92796716 | 26486 | 5: 92864262 |
| 26490 | 5: 92953627 | 26493 | 5: 93100450 | 26512 | 5: 93520661 | 26515 | 5: 93603557 | 26517 | 5: 93614794 |
| 26539 | 5: 94115151 | 26543 | 5: 94190519 | 26550 | 5: 94345725 | 26551 | 5: 94346916 | 26555 | 5: 94480126 |
| 26560 | 5: 94697047 | 26567 | 5: 94796315 | 26568 | 5: 94806990 | 26586 | 5: 95221444 | 26588 | 5: 95236526 |
| 26590 | 5: 95266607 | 26592 | 5: 95275712 | 26609 | 5: 95732385 | 26613 | 5: 95783150 | 26623 | 5: 96041063 |
| 26630 | 5: 96115730 | 26634 | 5: 96229534 | 26636 | 5: 96266124 | 26641 | 5: 96344421 | 26648 | 5: 96479304 |
| 26650 | 5: 96555471 | 26653 | 5: 96570693 | 26654 | 5: 96587114 | 26657 | 5: 96683112 | 26658 | 5: 96697638 |
| 26659 | 5: 96722002 | 26663 | 5: 96826963 | 26665 | 5: 96891834 | 26666 | 5: 96895092 | 26667 | 5: 96897342 |
| 26668 | 5: 96903857 | 26669 | 5: 96904695 | 26670 | 5: 96905308 | 26672 | 5: 96908444 | 26673 | 5: 96909315 |
| 26678 | 5: 96986050 | 26682 | 5: 97026543 | 26686 | 5: 97080334 | 26696 | 5: 97322489 | 26701 | 5: 97423919 |
| 26702 | 5: 97434109 | 26704 | 5: 97466504 | 26705 | 5: 97487627 | 26710 | 5: 97624543 | 26727 | 5: 98048878 |
| 26731 | 5: 98072135 | 26741 | 5: 98284200 | 26743 | 5: 98321751 | 26746 | 5: 98348925 | 26747 | 5: 98360313 |
| 26748 | 5: 98369761 | 26753 | 5: 98558774 | 26754 | 5: 98558789 | 26755 | 5: 98558837 | 26756 | 5: 98558879 |
| 26757 | 5: 98559154 | 26758 | 5: 98559190 | 26759 | 5: 98559458 | 26760 | 5: 98559459 | 26762 | 5: 98571338 |
| 26769 | 5: 98750219 | 26771 | 5: 98819283 | 26774 | 5: 98902747 | 26779 | 5: 98952191 | 26782 | 5: 98988266 |
| 26783 | 5: 98991352 | 26786 | 5: 99023106 | 26793 | 5: 99430794 | 26800 | 5: 99597967 | 26801 | 5: 99613789 |
| 26803 | 5: 99657462 | 26804 | 5: 99657533 | 26805 | 5: 99657651 | 26806 | 5: 99657668 | 26808 | 5: 99674708 |
| 26809 | 5: 99678222 | 26811 | 5: 99698551 | 26812 | 5: 99698552 | 26815 | 5: 99764163 | 26817 | 5: 99773482 |
| 26819 | 5: 99784430 | 26827 | 5: 99916927 | 26837 | 5: 100075903 | 26838 | 5: 100085604 | 26839 | 5: 100089578 |
| 26841 | 5: 100109898 | 26842 | 5: 100126206 | 26843 | 5: 100133218 | 26846 | 5: 100234492 | 26848 | 5: 100300261 |
| 26849 | 5: 100311514 | 26850 | 5: 100341741 | 26852 | 5: 100377385 | 26854 | 5: 100381117 | 26856 | 5: 100389557 |
| 26858 | 5: 100426926 | 26866 | 5: 100696501 | 26867 | 5: 100696607 | 26868 | 5: 100698121 | 26869 | 5: 100698579 |
| 26872 | 5: 100728733 | 26881 | 5: 100884953 | 26901 | 5: 101256059 | 26903 | 5: 101355705 | 26904 | 5: 101371243 |
| 26906 | 5: 101513339 | 26911 | 5: 101595246 | 26915 | 5: 101614862 | 26916 | 5: 101616220 | 26927 | 5: 101872342 |
| 26929 | 5: 101932704 | 26930 | 5: 101932771 | 26942 | 5: 102469703 | 26956 | 5: 103101288 | 26957 | 5: 103104040 |
| 26959 | 5: 103119311 | 26960 | 5: 103119357 | 26961 | 5: 103119383 | 26962 | 5: 103119408 | 26963 | 5: 103119413 |
| 26965 | 5: 103127016 | 26969 | 5: 103180670 | 26970 | 5: 103181148 | 26971 | 5: 103186080 | 26972 | 5: 103186163 |
| 26975 | 5: 103229995 | 26976 | 5: 103292486 | 26977 | 5: 103293544 | 26979 | 5: 103324283 | 26987 | 5: 103577479 |
| 26992 | 5: 103749650 | 26993 | 5: 103753540 | 26994 | 5: 103765906 | 27009 | 5: 104024017 | 27010 | 5: 104047698 |
| 27011 | 5: 104049926 | 27012 | 5: 104051208 | 27014 | 5: 104058009 | 27015 | 5: 104067878 | 27016 | 5: 104090665 |
| 27019 | 5: 104166543 | 27022 | 5: 104306581 | 27027 | 5: 104414866 | 27028 | 5: 104421461 | 27029 | 5: 104425156 |
| 27030 | 5: 104425652 | 27035 | 5: 104475838 | 27038 | 5: 104487779 | 27048 | 5: 104725697 | 27052 | 5: 104812593 |
| 27057 | 5: 104944974 | 27059 | 5: 104962040 | 27065 | 5: 105070263 | 27066 | 5: 105095351 | 27070 | 5: 105138348 |
| 27071 | 5: 105144347 | 27088 | 5: 105596087 | 27097 | 5: 105756724 | 27103 | 5: 105773809 | 27107 | 5: 105778777 |
| 27108 | 5: 105779368 | 27151 | 5: 106320490 | 27154 | 5: 106351560 | 27160 | 5: 106474477 | 27171 | 5: 106583604 |
| 27173 | 5: 106588115 | 27177 | 5: 106701615 | 27184 | 5: 106816406 | 27190 | 5: 106904200 | 27191 | 5: 106904337 |
| 27192 | 5: 106904427 | 27193 | 5: 106904456 | 27194 | 5: 106906152 | 27196 | 5: 106921443 | 27198 | 5: 106952814 |
| 27199 | 5: 106952815 | 27208 | 5: 107090339 | 27210 | 5: 107152886 | 27211 | 5: 107155008 | 27213 | 5: 107195685 |
| 27214 | 5: 107203743 | 27226 | 5: 107421295 | 27230 | 5: 107445396 | 27236 | 5: 107578561 | 27239 | 5: 107683951 |
| 27242 | 5: 107725814 | 27258 | 5: 108050852 | 27260 | 5: 108055831 | 27261 | 5: 108055990 | 27264 | 5: 108099551 |
| 27266 | 5: 108207411 | 27267 | 5: 108207896 | 27270 | 5: 108230364 | 27272 | 5: 108235905 | 27277 | 5: 108323867 |
| 27296 | 5: 108547597 | 27312 | 5: 108942683 | 27314 | 5: 108955049 | 27316 | 5: 108984906 | 27320 | 5: 109034391 |
| 27322 | 5: 109075632 | 27323 | 5: 109090548 | 27324 | 5: 109096526 | 27325 | 5: 109153205 | 27327 | 5: 109154168 |
| 27328 | 5: 109178144 | 27329 | 5: 109178358 | 27330 | 5: 109179308 | 27335 | 5: 109320605 | 27339 | 5: 109412747 |
| 27348 | 5: 109611685 | 27349 | 5: 109615284 | 27352 | 5: 109687895 | 27354 | 5: 109722487 | 27356 | 5: 109749503 |
| 27357 | 5: 109754096 | 27361 | 5: 109808607 | 27362 | 5: 109847158 | 27368 | 5: 109988776 | 27369 | 5: 109989550 |
| 27371 | 5: 110069248 | 27375 | 5: 110112327 | 27379 | 5: 110193224 | 27383 | 5: 110314027 | 27384 | 5: 110353226 |
| 27385 | 5: 110369778 | 27386 | 5: 110373456 | 27387 | 5: 110373555 | 27389 | 5: 110374859 | 27393 | 5: 110398433 |
| 27397 | 5: 110509525 | 27400 | 5: 110531320 | 27401 | 5: 110531367 | 27402 | 5: 110532648 | 27403 | 5: 110532703 |
| 27404 | 5: 110532709 | 27405 | 5: 110533852 | 27406 | 5: 110533859 | 27407 | 5: 110534455 | 27410 | 5: 110564870 |
| 27412 | 5: 110569021 | 27413 | 5: 110569141 | 27414 | 5: 110569146 | 27419 | 5: 110751512 | 27431 | 5: 111053132 |
| 27433 | 5: 111063936 | 27434 | 5: 111065855 | 27435 | 5: 111065875 | 27446 | 5: 111321416 | 27452 | 5: 111501396 |
| 27453 | 5: 111502809 | 27460 | 5: 111753901 | 27465 | 5: 111845063 | 27468 | 5: 111862953 | 27469 | 5: 111864267 |
| 27480 | 5: 112115889 | 27481 | 5: 112115946 | 27482 | 5: 112126293 | 27483 | 5: 112126298 | 27484 | 5: 112133653 |
| 27485 | 5: 112135929 | 27489 | 5: 112235754 | 27492 | 5: 112343380 | 27495 | 5: 112408202 | 27498 | 5: 112448429 |
| 27499 | 5: 112455682 | 27505 | 5: 112604562 | 27507 | 5: 112634025 | 27508 | 5: 112641994 | 27510 | 5: 112722034 |
| 27513 | 5: 112805426 | 27516 | 5: 112917350 | 27527 | 5: 113148146 | 27528 | 5: 113148464 | 27529 | 5: 113149795 |
| 27550 | 5: 113769146 | 27556 | 5: 113920144 | 27558 | 5: 113928017 | 27564 | 5: 114059697 | 27573 | 5: 114343422 |
| 27574 | 5: 114343454 | 27577 | 5: 114569421 | 27581 | 5: 114723102 | 27590 | 5: 115002143 | 27593 | 5: 115052949 |
| 27600 | 5: 115255504 | 27601 | 5: 115265500 | 27608 | 5: 115466163 | 27615 | 5: 115514965 | 27616 | 5: 115517449 |
| 27626 | 5: 115682503 | 27629 | 5: 115766557 | 27651 | 5: 116121476 | 27661 | 5: 116390999 | 27663 | 5: 116423647 |
| 27665 | 5: 116464508 | 27669 | 5: 116511553 | 27672 | 5: 116537986 | 27674 | 5: 116577132 | 27685 | 5: 116593122 |
| 27686 | 5: 116593191 | 27687 | 5: 116593192 | 27689 | 5: 116593706 | 27690 | 5: 116593715 | 27691 | 5: 116593762 |
| 27692 | 5: 116593794 | 27693 | 5: 116593850 | 27703 | 5: 116596428 | 27704 | 5: 116596441 | 27705 | 5: 116596625 |
| 27709 | 5: 116597945 | 27710 | 5: 116598050 | 27713 | 5: 116600211 | 27714 | 5: 116601776 | 27717 | 5: 116622560 |
| 27723 | 5: 116699001 | 27724 | 5: 116699079 | 27725 | 5: 116699138 | 27726 | 5: 116699145 | 27727 | 5: 116699159 |
| 27732 | 5: 116743643 | 27734 | 5: 116745570 | 27735 | 5: 116757705 | 27740 | 5: 116908657 | 27741 | 5: 116911216 |
| 27742 | 5: 116911371 | 27743 | 5: 116911376 | 27744 | 5: 116915304 | 27745 | 5: 116915313 | 27746 | 5: 116915402 |
| 27748 | 5: 116922121 | 27751 | 5: 116973879 | 27753 | 5: 116989243 | 27754 | 5: 116991259 | 27762 | 5: 117097690 |
| 27767 | 5: 117234102 | 27769 | 5: 117262549 | 27770 | 5: 117262555 | 27771 | 5: 117262556 | 27773 | 5: 117262630 |
| 27785 | 5: 117459589 | 27786 | 5: 117469696 | 27787 | 5: 117471979 | 27788 | 5: 117473591 | 27789 | 5: 117474224 |
| 27790 | 5: 117475215 | 27791 | 5: 117476538 | 27799 | 5: 117579793 | 27805 | 5: 117645005 | 27815 | 5: 117785747 |
| 27818 | 5: 117850396 | 27844 | 5: 118401624 | 27854 | 5: 118620070 | 27859 | 5: 118696815 | 27875 | 5: 118913308 |
| 27879 | 5: 118954058 | 27880 | 5: 118959454 | 27881 | 5: 118971379 | 27882 | 5: 118972496 | 27883 | 5: 118978705 |
| 27885 | 5: 118982714 | 27900 | 5: 119181604 | 27915 | 5: 119570841 | 27918 | 5: 119667915 | 27921 | 5: 119771063 |
| 27922 | 5: 119774093 | 27923 | 5: 119775410 | 27926 | 5: 119853084 | 27931 | 5: 119945721 | 27932 | 5: 119995446 |
| 27964 | 6: 844150 | 27965 | 6: 844153 | 27972 | 6: 1031446 | 27980 | 6: 1285962 | 27990 | 6: 1702526 |
| 27992 | 6: 1761466 | 27993 | 6: 1761542 | 27994 | 6: 1761563 | 27995 | 6: 1774727 | 27998 | 6: 1945674 |
| 28030 | 6: 2057409 | 28044 | 6: 2394123 | 28046 | 6: 2394185 | 28047 | 6: 2418816 | 28048 | 6: 2418834 |
| 28054 | 6: 2624789 | 28055 | 6: 2625544 | 28058 | 6: 2663078 | 28059 | 6: 2672214 | 28077 | 6: 3108815 |
| 28078 | 6: 3108854 | 28087 | 6: 3376371 | 28088 | 6: 3380776 | 28089 | 6: 3383123 | 28090 | 6: 3386603 |
| 28091 | 6: 3388251 | 28092 | 6: 3392342 | 28094 | 6: 3398350 | 28095 | 6: 3399218 | 28096 | 6: 3399858 |
| 28097 | 6: 3402165 | 28099 | 6: 3423360 | 28100 | 6: 3425355 | 28102 | 6: 3444538 | 28104 | 6: 3449326 |
| 28106 | 6: 3469199 | 28110 | 6: 3575704 | 28111 | 6: 3585145 | 28112 | 6: 3587923 | 28113 | 6: 3587933 |
| 28114 | 6: 3588243 | 28115 | 6: 3588261 | 28116 | 6: 3589475 | 28117 | 6: 3589994 | 28124 | 6: 3680340 |
| 28142 | 6: 4189994 | 28143 | 6: 4197131 | 28147 | 6: 4297910 | 28149 | 6: 4300166 | 28179 | 6: 4998395 |
| 28183 | 6: 5135323 | 28188 | 6: 5332029 | 28190 | 6: 5359913 | 28191 | 6: 5363670 | 28192 | 6: 5366571 |
| 28193 | 6: 5417516 | 28194 | 6: 5420554 | 28198 | 6: 5638260 | 28208 | 6: 5736468 | 28213 | 6: 5970478 |
| 28215 | 6: 6023720 | 28217 | 6: 6068366 | 28218 | 6: 6086349 | 28219 | 6: 6090367 | 28220 | 6: 6099226 |
| 28222 | 6: 6135400 | 28223 | 6: 6135571 | 28224 | 6: 6135977 | 28225 | 6: 6142753 | 28226 | 6: 6145385 |
| 28227 | 6: 6146291 | 28229 | 6: 6150122 | 28231 | 6: 6213823 | 28233 | 6: 6252521 | 28236 | 6: 6303620 |
| 28237 | 6: 6304251 | 28241 | 6: 6327370 | 28246 | 6: 6434692 | 28248 | 6: 6445175 | 28251 | 6: 6505947 |
| 28252 | 6: 6508802 | 28253 | 6: 6508845 | 28256 | 6: 6598639 | 28257 | 6: 6630222 | 28259 | 6: 6653707 |
| 28260 | 6: 6653733 | 28261 | 6: 6653820 | 28262 | 6: 6654166 | 28263 | 6: 6654264 | 28265 | 6: 6674156 |
| 28266 | 6: 6675058 | 28296 | 6: 7438091 | 28297 | 6: 7438117 | 28306 | 6: 7840060 | 28315 | 6: 8178961 |
| 28325 | 6: 8498664 | 28330 | 6: 8732227 | 28331 | 6: 8782400 | 28338 | 6: 8951172 | 28340 | 6: 8957062 |
| 28344 | 6: 8978291 | 28348 | 6: 8993893 | 28359 | 6: 9166519 | 28360 | 6: 9196740 | 28361 | 6: 9200024 |
| 28362 | 6: 9200854 | 28363 | 6: 9201774 | 28364 | 6: 9203527 | 28365 | 6: 9204459 | 28366 | 6: 9205411 |
| 28367 | 6: 9207235 | 28368 | 6: 9208280 | 28369 | 6: 9209206 | 28411 | 6: 10509217 | 28417 | 6: 10679784 |
| 28436 | 6: 11171281 | 28444 | 6: 11394489 | 28448 | 6: 11465975 | 28461 | 6: 11714410 | 28468 | 6: 11859669 |
| 28469 | 6: 11867154 | 28471 | 6: 11887100 | 28489 | 6: 12266091 | 28492 | 6: 12331408 | 28493 | 6: 12406590 |
| 28496 | 6: 12449553 | 28499 | 6: 12460758 | 28505 | 6: 12583026 | 28520 | 6: 12951400 | 28523 | 6: 13000409 |
| 28524 | 6: 13001855 | 28525 | 6: 13027415 | 28528 | 6: 13118294 | 28531 | 6: 13124831 | 28532 | 6: 13124859 |
| 28533 | 6: 13124885 | 28539 | 6: 13212064 | 28550 | 6: 13497569 | 28554 | 6: 13707108 | 28566 | 6: 14075733 |
| 28572 | 6: 14295399 | 28592 | 6: 14776571 | 28593 | 6: 14795242 | 28596 | 6: 14878264 | 28598 | 6: 14937683 |
| 28614 | 6: 15271409 | 28615 | 6: 15279031 | 28628 | 6: 15511470 | 28631 | 6: 15552360 | 28632 | 6: 15552836 |
| 28633 | 6: 15552929 | 28634 | 6: 15554381 | 28635 | 6: 15554470 | 28639 | 6: 15665547 | 28640 | 6: 15665565 |
| 28644 | 6: 15724964 | 28659 | 6: 16024226 | 28672 | 6: 16407876 | 28675 | 6: 16429738 | 28676 | 6: 16431811 |
| 28680 | 6: 16480595 | 28681 | 6: 16483148 | 28688 | 6: 16631779 | 28690 | 6: 16706078 | 28692 | 6: 16770941 |
| 28701 | 6: 16924337 | 28702 | 6: 16926868 | 28704 | 6: 16957756 | 28710 | 6: 17056311 | 28711 | 6: 17056315 |
| 28712 | 6: 17080889 | 28734 | 6: 17680046 | 28737 | 6: 17695292 | 28746 | 6: 17927268 | 28747 | 6: 17931957 |
| 28748 | 6: 17938149 | 28761 | 6: 18039717 | 28772 | 6: 18212765 | 28777 | 6: 18284502 | 28778 | 6: 18314365 |
| 28789 | 6: 18646895 | 28790 | 6: 18701849 | 28791 | 6: 18731749 | 28799 | 6: 18864835 | 28807 | 6: 19042586 |
| 28809 | 6: 19074411 | 28811 | 6: 19074485 | 28815 | 6: 19190465 | 28826 | 6: 19438867 | 28827 | 6: 19439848 |
| 28829 | 6: 19456967 | 28830 | 6: 19462913 | 28831 | 6: 19495081 | 28839 | 6: 19649377 | 28848 | 6: 19872402 |
| 28850 | 6: 19894311 | 28851 | 6: 19923403 | 28858 | 6: 20135992 | 28860 | 6: 20184822 | 28861 | 6: 20192872 |
| 28862 | 6: 20206522 | 28872 | 6: 20397821 | 28876 | 6: 20495916 | 28881 | 6: 20600966 | 28882 | 6: 20600990 |
| 28907 | 6: 21325769 | 28908 | 6: 21355207 | 28910 | 6: 21392641 | 28913 | 6: 21439632 | 28914 | 6: 21439691 |
| 28915 | 6: 21439763 | 28921 | 6: 21659954 | 28922 | 6: 21672266 | 28923 | 6: 21678873 | 28925 | 6: 21686069 |
| 28926 | 6: 21691325 | 28927 | 6: 21703290 | 28928 | 6: 21714263 | 28933 | 6: 21771563 | 28934 | 6: 21772453 |
| 28935 | 6: 21777544 | 28939 | 6: 21951701 | 28942 | 6: 22033622 | 28962 | 6: 22379732 | 28964 | 6: 22422233 |
| 28967 | 6: 22500214 | 28968 | 6: 22500233 | 28991 | 6: 23037086 | 28997 | 6: 23243928 | 29000 | 6: 23295269 |
| 29005 | 6: 23444102 | 29006 | 6: 23457488 | 29010 | 6: 23533175 | 29013 | 6: 23555860 | 29015 | 6: 23571729 |
| 29024 | 6: 23873913 | 29029 | 6: 23921917 | 29047 | 6: 24373068 | 29051 | 6: 24503230 | 29053 | 6: 24518463 |
| 29064 | 6: 24837558 | 29070 | 6: 24942515 | 29071 | 6: 24948898 | 29076 | 6: 25007714 | 29077 | 6: 25024841 |
| 29078 | 6: 25039222 | 29083 | 6: 25164226 | 29095 | 6: 25528726 | 29100 | 6: 25695588 | 29102 | 6: 25788477 |
| 29108 | 6: 25887098 | 29117 | 6: 26022919 | 29118 | 6: 26033719 | 29127 | 6: 26200013 | 29129 | 6: 26282778 |
| 29130 | 6: 26290561 | 29139 | 6: 26655531 | 29140 | 6: 26655687 | 29142 | 6: 26679658 | 29163 | 6: 27215526 |
| 29167 | 6: 27355784 | 29174 | 6: 27472557 | 29186 | 6: 27864310 | 29212 | 6: 28647979 | 29231 | 6: 29128064 |
| 29237 | 6: 29212166 | 29239 | 6: 29328156 | 29240 | 6: 29328334 | 29241 | 6: 29328350 | 29243 | 6: 29376641 |
| 29245 | 6: 29389036 | 29247 | 6: 29435290 | 29253 | 6: 29483476 | 29259 | 6: 29565346 | 29265 | 6: 29684500 |
| 29279 | 6: 30075635 | 29342 | 6: 30145040 | 29365 | 6: 30563336 | 29399 | 6: 31576983 | 29412 | 6: 31922816 |
| 29421 | 6: 32195477 | 29425 | 6: 32453198 | 29426 | 6: 32493429 | 29468 | 6: 34036502 | 29472 | 6: 34067879 |
| 29473 | 6: 34068521 | 29486 | 6: 34217305 | 29587 | 6: 34344002 | 29660 | 6: 34467878 | 29733 | 6: 34605510 |
| 29954 | 6: 35510819 | 29957 | 6: 35561016 | 29986 | 6: 35857940 | 30022 | 6: 36053886 | 30024 | 6: 36074911 |
| 30130 | 6: 36395104 | 30139 | 6: 36431315 | 30145 | 6: 36444505 | 30148 | 6: 36448732 | 30174 | 6: 36489959 |
| 30250 | 6: 36553066 | 30255 | 6: 36561452 | 30300 | 6: 36642083 | 30317 | 6: 36688082 | 30321 | 6: 36706370 |
| 30383 | 6: 36857123 | 30808 | 6: 37218423 | 31143 | 6: 37396789 | 31963 | 6: 39186781 | 31964 | 6: 39249570 |
| 31972 | 6: 39371669 | 31973 | 6: 39392848 | 31997 | 6: 39755061 | 32033 | 6: 40227846 | 32038 | 6: 40262465 |
| 32095 | 6: 40813933 | 32099 | 6: 40846711 | 32101 | 6: 40869544 | 32162 | 6: 41204988 | 32163 | 6: 41205018 |
| 32194 | 6: 41998863 | 32195 | 6: 42004652 | 32196 | 6: 42005216 | 32197 | 6: 42010829 | 32198 | 6: 42011964 |
| 32199 | 6: 42014380 | 32200 | 6: 42017579 | 32202 | 6: 42030911 | 32206 | 6: 42098249 | 32238 | 6: 42816146 |
| 32247 | 6: 42950720 | 32272 | 6: 43082880 | 32273 | 6: 43090582 | 32274 | 6: 43121398 | 32288 | 6: 43377340 |
| 32289 | 6: 43378488 | 32310 | 6: 43688837 | 32324 | 6: 43887416 | 32334 | 6: 44040805 | 32335 | 6: 44059941 |
| 32336 | 6: 44072335 | 32349 | 6: 44361206 | 32359 | 6: 44628166 | 32367 | 6: 44692016 | 32483 | 6: 45118837 |
| 32493 | 6: 45218126 | 32498 | 6: 45277318 | 32501 | 6: 45322457 | 32503 | 6: 45335602 | 32504 | 6: 45364598 |
| 32507 | 6: 45390455 | 32509 | 6: 45416952 | 32513 | 6: 45505416 | 32514 | 6: 45522021 | 32520 | 6: 45688164 |
| 32531 | 6: 45935668 | 32532 | 6: 45982939 | 32534 | 6: 46022376 | 32535 | 6: 46078755 | 32545 | 6: 46209779 |
| 32550 | 6: 46337526 | 32552 | 6: 46378480 | 32553 | 6: 46389387 | 32554 | 6: 46390042 | 32577 | 6: 46954860 |
| 32582 | 6: 47076276 | 32595 | 6: 47353083 | 32606 | 6: 47640345 | 32616 | 6: 47866852 | 32618 | 6: 47903812 |
| 32622 | 6: 48018241 | 32634 | 6: 48398260 | 32642 | 6: 48750204 | 32664 | 6: 49330544 | 32666 | 6: 49366546 |
| 32681 | 6: 49731100 | 32682 | 6: 49732769 | 32683 | 6: 49734879 | 32684 | 6: 49738480 | 32685 | 6: 49759413 |
| 32686 | 6: 49766766 | 32687 | 6: 49773026 | 32690 | 6: 49804879 | 32691 | 6: 49816719 | 32697 | 6: 49895642 |
| 32705 | 6: 50190150 | 32709 | 6: 50252554 | 32711 | 6: 50316194 | 32712 | 6: 50316384 | 32713 | 6: 50316707 |
| 32714 | 6: 50342873 | 32745 | 6: 51287866 | 32761 | 6: 51779722 | 32770 | 6: 52097852 | 32771 | 6: 52097941 |
| 32776 | 6: 52230394 | 32777 | 6: 52230455 | 32779 | 6: 52258121 | 32811 | 6: 52492036 | 32823 | 6: 52776675 |
| 32839 | 6: 53189333 | 32841 | 6: 53215914 | 32845 | 6: 53260563 | 32856 | 6: 53542273 | 32858 | 6: 53587223 |
| 32871 | 6: 53982975 | 32882 | 6: 54231133 | 32895 | 6: 54705767 | 32905 | 6: 55072307 | 32957 | 6: 55697219 |
| 32965 | 6: 55964167 | 32981 | 6: 56569534 | 32982 | 6: 56580948 | 32997 | 6: 56927864 | 32998 | 6: 56927881 |
| 32999 | 6: 56927920 | 33000 | 6: 56928100 | 33011 | 6: 57258298 | 33013 | 6: 57310043 | 33016 | 6: 57383139 |
| 33019 | 6: 57452741 | 33021 | 6: 57538068 | 33024 | 6: 57589915 | 33053 | 6: 58398195 | 33055 | 6: 58507178 |
| 33065 | 6: 58716078 | 33075 | 6: 59092959 | 33080 | 6: 59188156 | 33085 | 6: 59262762 | 33100 | 6: 59749739 |
| 33108 | 6: 59853909 | 33118 | 6: 59966318 | 33123 | 6: 60097949 | 33128 | 6: 60226786 | 33130 | 6: 60265173 |
| 33131 | 6: 60301627 | 33133 | 6: 60345603 | 33135 | 6: 60373758 | 33138 | 6: 60422003 | 33140 | 6: 60468668 |
| 33152 | 6: 60773644 | 33166 | 6: 61158905 | 33167 | 6: 61161272 | 33172 | 6: 61280689 | 33177 | 6: 61397201 |
| 33179 | 6: 61410508 | 33180 | 6: 61421440 | 33187 | 6: 61548278 | 33188 | 6: 61582228 | 33189 | 6: 61589076 |
| 33190 | 6: 61673425 | 33217 | 6: 62603953 | 33226 | 6: 62889998 | 33231 | 6: 62988117 | 33232 | 6: 63010364 |
| 33233 | 6: 63010380 | 33235 | 6: 63029593 | 33237 | 6: 63097017 | 33247 | 6: 63307538 | 33248 | 6: 63308671 |
| 33249 | 6: 63347756 | 33269 | 6: 64151594 | 33281 | 6: 64662329 | 33282 | 6: 64665326 | 33293 | 6: 65016085 |
| 33300 | 6: 65345032 | 33302 | 6: 65383049 | 33304 | 6: 65420404 | 33305 | 6: 65422497 | 33306 | 6: 65422592 |
| 33307 | 6: 65456807 | 33325 | 6: 66109765 | 33332 | 6: 66343250 | 33334 | 6: 66387388 | 33340 | 6: 66651619 |
| 33345 | 6: 66831939 | 33348 | 6: 66876573 | 33353 | 6: 66988792 | 33356 | 6: 67035579 | 33358 | 6: 67089021 |
| 33373 | 6: 67582129 | 33382 | 6: 68017233 | 33387 | 6: 68177405 | 33390 | 6: 68268998 | 33395 | 6: 68352179 |
| 33401 | 6: 68565298 | 33402 | 6: 68589087 | 33403 | 6: 68598341 | 33410 | 6: 68728215 | 33413 | 6: 68763773 |
| 33415 | 6: 68828972 | 33417 | 6: 68885563 | 33420 | 6: 68935879 | 33426 | 6: 69138647 | 33428 | 6: 69168309 |
| 33430 | 6: 69216815 | 33434 | 6: 69287580 | 33436 | 6: 69352586 | 33439 | 6: 69439499 | 33445 | 6: 69755083 |
| 33452 | 6: 69895326 | 33464 | 6: 70383634 | 33465 | 6: 70399966 | 33466 | 6: 70404486 | 33467 | 6: 70405959 |
| 33468 | 6: 70423245 | 33472 | 6: 70564450 | 33481 | 6: 70877482 | 33482 | 6: 70900974 | 33490 | 6: 71126939 |
| 33494 | 6: 71294876 | 33504 | 6: 71562156 | 33510 | 6: 71703879 | 33518 | 6: 72060315 | 33522 | 6: 72174384 |
| 33531 | 6: 72351300 | 33533 | 6: 72379846 | 33540 | 6: 72542846 | 33548 | 6: 72783452 | 33558 | 6: 73223485 |
| 33565 | 6: 73414475 | 33567 | 6: 73481332 | 33570 | 6: 73620191 | 33577 | 6: 73801067 | 33631 | 6: 75798192 |
| 33645 | 6: 76545846 | 33646 | 6: 76546983 | 33647 | 6: 76547862 | 33665 | 6: 76855629 | 33677 | 6: 77240842 |
| 33680 | 6: 77349471 | 33691 | 6: 77688509 | 33694 | 6: 77787079 | 33711 | 6: 78425706 | 33712 | 6: 78429257 |
| 33717 | 6: 78761621 | 33718 | 6: 78761714 | 33721 | 6: 78917750 | 33743 | 6: 79765417 | 33765 | 6: 80471442 |
| 33766 | 6: 80482749 | 33767 | 6: 80484712 | 33768 | 6: 80491703 | 33769 | 6: 80530130 | 33770 | 6: 80567928 |
| 33771 | 6: 80590178 | 33773 | 6: 80675168 | 33788 | 6: 81051057 | 33814 | 6: 82117911 | 33855 | 6: 83277672 |
| 33858 | 6: 83308141 | 33861 | 6: 83347263 | 33864 | 6: 83451575 | 33865 | 6: 83451628 | 33866 | 6: 83451712 |
| 33867 | 6: 83451781 | 33868 | 6: 83451862 | 33870 | 6: 83469477 | 33871 | 6: 83470850 | 33872 | 6: 83475021 |
| 33884 | 6: 83758356 | 33885 | 6: 83763398 | 33892 | 6: 83946894 | 33893 | 6: 83948279 | 33894 | 6: 83950501 |
| 33898 | 6: 83985933 | 33902 | 6: 84087846 | 33903 | 6: 84138032 | 33910 | 6: 84497175 | 33911 | 6: 84497631 |
| 33912 | 6: 84497709 | 33913 | 6: 84511587 | 33914 | 6: 84511656 | 33915 | 6: 84513009 | 33932 | 6: 84975132 |
| 33933 | 6: 85067620 | 33934 | 6: 85092477 | 33935 | 6: 85092499 | 33936 | 6: 85092523 | 33937 | 6: 85093071 |
| 33938 | 6: 85108153 | 33939 | 6: 85108165 | 33940 | 6: 85108432 | 33941 | 6: 85108773 | 33943 | 6: 85136420 |
| 33944 | 6: 85137180 | 33950 | 6: 85315912 | 33977 | 6: 85896040 | 33978 | 6: 85896097 | 33979 | 6: 85896105 |
| 33980 | 6: 85896139 | 33981 | 6: 85906390 | 33982 | 6: 85908497 | 34003 | 6: 86508590 | 34012 | 6: 86710155 |
| 34028 | 6: 86901499 | 34032 | 6: 86989414 | 34034 | 6: 87007077 | 34040 | 6: 87076603 | 34043 | 6: 87111828 |
| 34057 | 6: 87463348 | 34067 | 6: 87603473 | 34074 | 6: 87731662 | 34082 | 6: 87796536 | 34085 | 6: 87923433 |
| 34086 | 6: 87923536 | 34092 | 6: 88075544 | 34096 | 6: 88164435 | 34097 | 6: 88184411 | 34098 | 6: 88184633 |
| 34102 | 6: 88318314 | 34108 | 6: 88486850 | 34116 | 6: 88859149 | 34130 | 6: 89152164 | 34131 | 6: 89181036 |
| 34150 | 6: 89663234 | 34152 | 6: 89663319 | 34153 | 6: 89663348 | 34154 | 6: 89674618 | 34162 | 6: 89768711 |
| 34170 | 6: 89975431 | 34182 | 6: 90390426 | 34185 | 6: 90416932 | 34186 | 6: 90457703 | 34196 | 6: 90680946 |
| 34199 | 6: 90714640 | 34200 | 6: 90726127 | 34206 | 6: 90794035 | 34208 | 6: 90816980 | 34210 | 6: 90823124 |
| 34211 | 6: 90826546 | 34212 | 6: 90826598 | 34213 | 6: 90827159 | 34214 | 6: 90836066 | 34215 | 6: 90836072 |
| 34216 | 6: 90836166 | 34221 | 6: 90946704 | 34228 | 6: 91105837 | 34229 | 6: 91122456 | 34236 | 6: 91204532 |
| 34237 | 6: 91223593 | 34238 | 6: 91226875 | 34239 | 6: 91246239 | 34240 | 6: 91275706 | 34241 | 6: 91282834 |
| 34242 | 6: 91284101 | 34243 | 6: 91284414 | 34244 | 6: 91285011 | 34245 | 6: 91285694 | 34246 | 6: 91285851 |
| 34247 | 6: 91285925 | 34248 | 6: 91290335 | 34249 | 6: 91294383 | 34250 | 6: 91322998 | 34257 | 6: 91406353 |
| 34258 | 6: 91408596 | 34260 | 6: 91433962 | 34261 | 6: 91462732 | 34271 | 6: 91615288 | 34272 | 6: 91616087 |
| 34275 | 6: 91649638 | 34277 | 6: 91692231 | 34280 | 6: 91736404 | 34282 | 6: 91751612 | 34284 | 6: 91836628 |
| 34287 | 6: 91878223 | 34291 | 6: 91994872 | 34300 | 6: 92336028 | 34302 | 6: 92383295 | 34308 | 6: 92595572 |
| 34310 | 6: 92678382 | 34313 | 6: 92774897 | 34317 | 6: 92832688 | 34335 | 6: 93291707 | 34336 | 6: 93291712 |
| 34337 | 6: 93291776 | 34343 | 6: 93354640 | 34344 | 6: 93363500 | 34351 | 6: 93496619 | 34353 | 6: 93533733 |
| 34361 | 6: 93646233 | 34373 | 6: 93947051 | 34378 | 6: 94069815 | 34389 | 6: 94150648 | 34392 | 6: 94188032 |
| 34393 | 6: 94201168 | 34394 | 6: 94204543 | 34395 | 6: 94215369 | 34401 | 6: 94394468 | 34403 | 6: 94416755 |
| 34413 | 6: 94854202 | 34424 | 6: 95339487 | 34426 | 6: 95406474 | 34427 | 6: 95406527 | 34429 | 6: 95406556 |
| 34432 | 6: 95553456 | 34441 | 6: 95787499 | 34444 | 6: 95884565 | 34449 | 6: 96032153 | 34451 | 6: 96087038 |
| 34462 | 6: 96247005 | 34474 | 6: 96452752 | 34496 | 6: 97018222 | 34504 | 6: 97179175 | 34505 | 6: 97179176 |
| 34506 | 6: 97179179 | 34507 | 6: 97179203 | 34508 | 6: 97179214 | 34509 | 6: 97179248 | 34510 | 6: 97217522 |
| 34511 | 6: 97226901 | 34512 | 6: 97244565 | 34513 | 6: 97244652 | 34514 | 6: 97244662 | 34515 | 6: 97255320 |
| 34516 | 6: 97255455 | 34517 | 6: 97255461 | 34528 | 6: 97447881 | 34529 | 6: 97449063 | 34530 | 6: 97453689 |
| 34544 | 6: 97650137 | 34547 | 6: 97742989 | 34556 | 6: 98047323 | 34558 | 6: 98052606 | 34566 | 6: 98256336 |
| 34570 | 6: 98334890 | 34571 | 6: 98337762 | 34572 | 6: 98337909 | 34573 | 6: 98337919 | 34574 | 6: 98338489 |
| 34575 | 6: 98338596 | 34576 | 6: 98338640 | 34577 | 6: 98338644 | 34579 | 6: 98372826 | 34581 | 6: 98379884 |
| 34596 | 6: 98671599 | 34608 | 6: 98974175 | 34628 | 6: 99529167 | 34637 | 6: 99783201 | 34641 | 6: 99954538 |
| 34669 | 6: 100390632 | 34671 | 6: 100431779 | 34678 | 6: 100585876 | 34685 | 6: 100727406 | 34689 | 6: 100852199 |
| 34693 | 6: 100953221 | 34706 | 6: 101294567 | 34707 | 6: 101294597 | 34714 | 6: 101450871 | 34729 | 6: 101787815 |
| 34730 | 6: 101837470 | 34739 | 6: 101917014 | 34740 | 6: 101935968 | 34741 | 6: 101941367 | 34744 | 6: 101973857 |
| 34754 | 6: 102148393 | 34755 | 6: 102148443 | 34760 | 6: 102234263 | 34766 | 6: 102424852 | 34772 | 6: 102510343 |
| 34779 | 6: 102606444 | 34783 | 6: 102645168 | 34784 | 6: 102645192 | 34785 | 6: 102645198 | 34788 | 6: 102756289 |
| 34791 | 6: 102773226 | 34793 | 6: 102854233 | 34795 | 6: 102855258 | 34796 | 6: 102857313 | 34797 | 6: 102870507 |
| 34798 | 6: 102871524 | 34799 | 6: 102872128 | 34800 | 6: 102873595 | 34801 | 6: 102875781 | 34802 | 6: 102889726 |
| 34803 | 6: 102890450 | 34804 | 6: 102892630 | 34805 | 6: 102896127 | 34806 | 6: 102896985 | 34807 | 6: 102927406 |
| 34812 | 6: 103013214 | 34822 | 6: 103212156 | 34835 | 6: 103357734 | 34837 | 6: 103396597 | 34839 | 6: 103431771 |
| 34841 | 6: 103480885 | 34842 | 6: 103490310 | 34843 | 6: 103490339 | 34844 | 6: 103490470 | 34848 | 6: 103557818 |
| 34851 | 6: 103637428 | 34852 | 6: 103640406 | 34854 | 6: 103654616 | 34855 | 6: 103663732 | 34856 | 6: 103741666 |
| 34857 | 6: 103773464 | 34858 | 6: 103774096 | 34862 | 6: 103862052 | 34864 | 6: 103898267 | 34876 | 6: 104267666 |
| 34881 | 6: 104339504 | 34892 | 6: 104657713 | 34893 | 6: 104658492 | 34897 | 6: 104793010 | 34898 | 6: 104793414 |
| 34899 | 6: 104809492 | 34900 | 6: 104809647 | 34901 | 6: 104824268 | 34903 | 6: 104868243 | 34905 | 6: 104881408 |
| 34909 | 6: 104928796 | 34914 | 6: 105104413 | 34924 | 6: 105290637 | 34927 | 6: 105378109 | 34929 | 6: 105423924 |
| 34931 | 6: 105504062 | 34940 | 6: 105638984 | 34941 | 6: 105650631 | 34950 | 6: 105886896 | 34959 | 6: 106123065 |
| 34966 | 6: 106312210 | 34971 | 6: 106540140 | 34979 | 6: 106739419 | 34986 | 6: 106953368 | 35009 | 6: 107508591 |
| 35024 | 6: 108020924 | 35045 | 6: 108596491 | 35055 | 6: 108951951 | 35057 | 6: 108952102 | 35059 | 6: 108993006 |
| 35068 | 6: 109199682 | 35071 | 6: 109282471 | 35072 | 6: 109282486 | 35102 | 6: 109823696 | 35111 | 6: 110018683 |
| 35142 | 6: 110852081 | 35156 | 6: 111034643 | 35157 | 6: 111034669 | 35160 | 6: 111043273 | 35167 | 6: 111198616 |
| 35176 | 6: 111399485 | 35180 | 6: 111474948 | 35189 | 6: 111673792 | 35198 | 6: 111776075 | 35200 | 6: 111795733 |
| 35201 | 6: 111796366 | 35205 | 6: 111879814 | 35206 | 6: 111880060 | 35208 | 6: 111944736 | 35209 | 6: 111951572 |
| 35226 | 6: 112530579 | 35231 | 6: 112650810 | 35234 | 6: 112672985 | 35239 | 6: 112735477 | 35241 | 6: 112750366 |
| 35247 | 6: 112863762 | 35251 | 6: 112908010 | 35255 | 6: 112989544 | 35262 | 6: 113261157 | 35272 | 6: 113442820 |
| 35276 | 6: 113471367 | 35277 | 6: 113480954 | 35281 | 6: 113591450 | 35288 | 6: 113702935 | 35290 | 6: 113754589 |
| 35291 | 6: 113756287 | 35292 | 6: 113756982 | 35293 | 6: 113765934 | 35295 | 6: 113775951 | 35297 | 6: 113799562 |
| 35298 | 6: 113821699 | 35300 | 6: 113827124 | 35301 | 6: 113830450 | 35309 | 6: 113999468 | 35311 | 6: 114031327 |
| 35312 | 6: 114040384 | 35320 | 6: 114163874 | 35324 | 6: 114205337 | 35327 | 6: 114271753 | 35334 | 6: 114428994 |
| 35336 | 6: 114452241 | 35337 | 6: 114471056 | 35342 | 6: 114583005 | 35344 | 6: 114601159 | 35345 | 6: 114602046 |
| 35351 | 6: 114719008 | 35355 | 6: 114797024 | 35365 | 6: 115032289 | 35366 | 6: 115035590 | 35367 | 6: 115035592 |
| 35376 | 6: 115201677 | 35382 | 6: 115369888 | 35401 | 6: 115856852 | 35403 | 6: 115872479 | 35404 | 6: 115959886 |
| 35408 | 6: 116004094 | 35409 | 6: 116011104 | 35416 | 6: 116154808 | 35417 | 6: 116160046 | 35420 | 6: 116200574 |
| 35422 | 6: 116286341 | 35425 | 6: 116319165 | 35430 | 6: 116485262 | 35431 | 6: 116485283 | 35432 | 6: 116485335 |
| 35433 | 6: 116485360 | 35434 | 6: 116485372 | 35435 | 6: 116485909 | 35436 | 6: 116487900 | 35437 | 6: 116487922 |
| 35438 | 6: 116488922 | 35439 | 6: 116489686 | 35440 | 6: 116489801 | 35441 | 6: 116500010 | 35442 | 6: 116500063 |
| 35443 | 6: 116500364 | 35444 | 6: 116500379 | 35445 | 6: 116509870 | 35446 | 6: 116514508 | 35447 | 6: 116514529 |
| 35453 | 6: 116932235 | 35457 | 6: 117212852 | 35458 | 6: 117247186 | 35459 | 6: 117253860 | 35462 | 6: 117399949 |
| 35463 | 6: 117405411 | 35464 | 6: 117444202 | 35465 | 6: 117445801 | 35466 | 6: 117445935 | 35467 | 6: 117463206 |
| 35490 | 7: 516102 | 35502 | 7: 810744 | 35503 | 7: 822079 | 35533 | 7: 1434030 | 35534 | 7: 1439391 |
| 35535 | 7: 1444418 | 35537 | 7: 1508351 | 35539 | 7: 1552952 | 35546 | 7: 1663078 | 35547 | 7: 1663134 |
| 35550 | 7: 1706720 | 35553 | 7: 1757821 | 35560 | 7: 1977089 | 35562 | 7: 1996339 | 35563 | 7: 2036084 |
| 35567 | 7: 2112952 | 35577 | 7: 2352038 | 35587 | 7: 2461961 | 35590 | 7: 2556375 | 35594 | 7: 2628007 |
| 35595 | 7: 2628008 | 35596 | 7: 2628014 | 35597 | 7: 2628018 | 35598 | 7: 2628066 | 35599 | 7: 2628069 |
| 35600 | 7: 2628233 | 35601 | 7: 2628255 | 35602 | 7: 2628256 | 35604 | 7: 2630943 | 35605 | 7: 2630947 |
| 35606 | 7: 2631054 | 35607 | 7: 2631064 | 35610 | 7: 2682673 | 35611 | 7: 2682716 | 35612 | 7: 2682743 |
| 35613 | 7: 2682770 | 35614 | 7: 2682781 | 35615 | 7: 2682785 | 35618 | 7: 2721521 | 35619 | 7: 2728053 |
| 35622 | 7: 2823155 | 35624 | 7: 2880427 | 35629 | 7: 2957250 | 35631 | 7: 3004323 | 35641 | 7: 3142936 |
| 35643 | 7: 3239293 | 35670 | 7: 3679709 | 35671 | 7: 3679809 | 35672 | 7: 3686101 | 35673 | 7: 3687496 |
| 35683 | 7: 3876358 | 35687 | 7: 3947888 | 35688 | 7: 3949113 | 35689 | 7: 3953770 | 35690 | 7: 3965550 |
| 35692 | 7: 4018660 | 35693 | 7: 4057873 | 35700 | 7: 4183821 | 35702 | 7: 4203677 | 35704 | 7: 4226758 |
| 35706 | 7: 4321618 | 35708 | 7: 4425609 | 35709 | 7: 4432045 | 35713 | 7: 4509941 | 35714 | 7: 4530497 |
| 35715 | 7: 4550094 | 35724 | 7: 4890795 | 35725 | 7: 4931147 | 35733 | 7: 5056411 | 35734 | 7: 5076528 |
| 35735 | 7: 5077856 | 35736 | 7: 5077899 | 35737 | 7: 5077986 | 35738 | 7: 5078073 | 35743 | 7: 5135884 |
| 35744 | 7: 5135971 | 35747 | 7: 5227598 | 35758 | 7: 5539362 | 35759 | 7: 5540415 | 35760 | 7: 5541785 |
| 35761 | 7: 5542691 | 35762 | 7: 5543242 | 35763 | 7: 5543903 | 35764 | 7: 5544868 | 35765 | 7: 5545851 |
| 35766 | 7: 5546627 | 35767 | 7: 5547659 | 35768 | 7: 5548421 | 35769 | 7: 5554183 | 35770 | 7: 5554705 |
| 35771 | 7: 5555278 | 35772 | 7: 5556329 | 35773 | 7: 5560431 | 35775 | 7: 5587078 | 35781 | 7: 5670138 |
| 35783 | 7: 5725687 | 35784 | 7: 5728200 | 35787 | 7: 5774079 | 35788 | 7: 5775685 | 35789 | 7: 5775738 |
| 35790 | 7: 5775833 | 35791 | 7: 5775864 | 35794 | 7: 5854438 | 35795 | 7: 5867893 | 35796 | 7: 5869641 |
| 35797 | 7: 5875629 | 35798 | 7: 5876835 | 35799 | 7: 5879220 | 35800 | 7: 5884897 | 35801 | 7: 5888476 |
| 35803 | 7: 5895045 | 35804 | 7: 5905064 | 35807 | 7: 5948030 | 35839 | 7: 6133269 | 35848 | 7: 6256347 |
| 35850 | 7: 6276752 | 35852 | 7: 6318895 | 35853 | 7: 6318915 | 35867 | 7: 6376467 | 35868 | 7: 6389582 |
| 35877 | 7: 6654578 | 35885 | 7: 6739561 | 35892 | 7: 6820748 | 35896 | 7: 6837253 | 35905 | 7: 7044892 |
| 35906 | 7: 7044893 | 35909 | 7: 7081346 | 35912 | 7: 7131477 | 35913 | 7: 7131577 | 35979 | 7: 9765475 |
| 35998 | 7: 10641237 | 36009 | 7: 11092130 | 36015 | 7: 11145935 | 36016 | 7: 11154819 | 36019 | 7: 11233180 |
| 36020 | 7: 11233207 | 36021 | 7: 11237049 | 36022 | 7: 11296724 | 36023 | 7: 11306644 | 36024 | 7: 11332158 |
| 36026 | 7: 11394630 | 36027 | 7: 11394736 | 36028 | 7: 11403649 | 36042 | 7: 11870658 | 36043 | 7: 11885250 |
| 36062 | 7: 12227428 | 36070 | 7: 12344743 | 36071 | 7: 12344879 | 36082 | 7: 12522109 | 36088 | 7: 12587487 |
| 36093 | 7: 12738904 | 36096 | 7: 12762542 | 36097 | 7: 12763942 | 36099 | 7: 12806635 | 36100 | 7: 12816543 |
| 36102 | 7: 12824241 | 36103 | 7: 12852855 | 36106 | 7: 12896445 | 36107 | 7: 12897971 | 36108 | 7: 12898039 |
| 36109 | 7: 12898062 | 36111 | 7: 12974218 | 36112 | 7: 12974361 | 36115 | 7: 13016195 | 36121 | 7: 13189985 |
| 36122 | 7: 13201701 | 36123 | 7: 13204223 | 36124 | 7: 13204306 | 36125 | 7: 13209001 | 36126 | 7: 13209008 |
| 36127 | 7: 13209051 | 36128 | 7: 13209093 | 36129 | 7: 13211564 | 36130 | 7: 13211698 | 36131 | 7: 13211746 |
| 36133 | 7: 13234671 | 36135 | 7: 13241201 | 36136 | 7: 13248867 | 36137 | 7: 13252997 | 36145 | 7: 13436247 |
| 36160 | 7: 13763740 | 36161 | 7: 13764174 | 36163 | 7: 13826339 | 36164 | 7: 13826607 | 36165 | 7: 13847719 |
| 36166 | 7: 13847770 | 36167 | 7: 13848168 | 36169 | 7: 13907560 | 36170 | 7: 13907747 | 36171 | 7: 13908190 |
| 36175 | 7: 13970540 | 36176 | 7: 13970551 | 36177 | 7: 13971039 | 36182 | 7: 14105363 | 36187 | 7: 14216180 |
| 36188 | 7: 14278452 | 36196 | 7: 14568342 | 36197 | 7: 14569870 | 36198 | 7: 14569910 | 36199 | 7: 14570835 |
| 36200 | 7: 14571116 | 36201 | 7: 14571263 | 36202 | 7: 14572010 | 36203 | 7: 14572312 | 36204 | 7: 14574790 |
| 36205 | 7: 14574794 | 36206 | 7: 14574824 | 36207 | 7: 14574906 | 36208 | 7: 14575332 | 36209 | 7: 14575373 |
| 36210 | 7: 14576408 | 36211 | 7: 14576429 | 36212 | 7: 14576444 | 36213 | 7: 14577168 | 36214 | 7: 14577625 |
| 36215 | 7: 14578417 | 36216 | 7: 14578461 | 36217 | 7: 14578495 | 36218 | 7: 14587075 | 36221 | 7: 14674502 |
| 36222 | 7: 14674589 | 36224 | 7: 14688808 | 36225 | 7: 14689037 | 36226 | 7: 14690346 | 36227 | 7: 14690813 |
| 36228 | 7: 14692758 | 36229 | 7: 14695086 | 36230 | 7: 14790790 | 36231 | 7: 14846055 | 36232 | 7: 14847442 |
| 36241 | 7: 15055421 | 36242 | 7: 15055460 | 36246 | 7: 15113333 | 36247 | 7: 15116085 | 36248 | 7: 15116605 |
| 36249 | 7: 15120550 | 36250 | 7: 15121347 | 36251 | 7: 15121459 | 36252 | 7: 15131143 | 36254 | 7: 15149591 |
| 36255 | 7: 15158037 | 36257 | 7: 15188314 | 36258 | 7: 15219440 | 36262 | 7: 15240130 | 36267 | 7: 15342910 |
| 36269 | 7: 15386114 | 36273 | 7: 15463697 | 36274 | 7: 15501033 | 36283 | 7: 15718347 | 36297 | 7: 16146481 |
| 36298 | 7: 16189628 | 36300 | 7: 16237783 | 36305 | 7: 16292747 | 36313 | 7: 16521674 | 36314 | 7: 16522621 |
| 36315 | 7: 16524699 | 36317 | 7: 16531552 | 36318 | 7: 16534211 | 36319 | 7: 16535641 | 36323 | 7: 16561541 |
| 36359 | 7: 17538355 | 36366 | 7: 17643702 | 36367 | 7: 17694868 | 36368 | 7: 17698407 | 36369 | 7: 17698410 |
| 36371 | 7: 17712921 | 36389 | 7: 18292860 | 36395 | 7: 18386812 | 36397 | 7: 18404440 | 36398 | 7: 18434206 |
| 36399 | 7: 18443286 | 36401 | 7: 18446615 | 36402 | 7: 18464757 | 36408 | 7: 18658963 | 36411 | 7: 18704573 |
| 36412 | 7: 18706088 | 36413 | 7: 18777233 | 36414 | 7: 18826386 | 36417 | 7: 18862062 | 36424 | 7: 19108968 |
| 36425 | 7: 19113510 | 36426 | 7: 19115360 | 36428 | 7: 19141191 | 36431 | 7: 19189844 | 36436 | 7: 19259287 |
| 36437 | 7: 19261276 | 36438 | 7: 19270709 | 36439 | 7: 19272732 | 36443 | 7: 19317767 | 36446 | 7: 19337660 |
| 36449 | 7: 19501368 | 36451 | 7: 19560991 | 36452 | 7: 19570534 | 36453 | 7: 19601934 | 36454 | 7: 19624357 |
| 36455 | 7: 19629075 | 36456 | 7: 19634135 | 36457 | 7: 19674365 | 36459 | 7: 19694033 | 36460 | 7: 19709157 |
| 36462 | 7: 19723388 | 36463 | 7: 19742259 | 36464 | 7: 19745894 | 36465 | 7: 19749906 | 36468 | 7: 19770884 |
| 36469 | 7: 19782434 | 36470 | 7: 19795534 | 36471 | 7: 19811128 | 36473 | 7: 19832477 | 36474 | 7: 19846412 |
| 36475 | 7: 19854110 | 36476 | 7: 19861073 | 36477 | 7: 19870302 | 36478 | 7: 19874067 | 36479 | 7: 19877997 |
| 36480 | 7: 19882846 | 36481 | 7: 19900064 | 36482 | 7: 19904145 | 36483 | 7: 19920619 | 36484 | 7: 19925859 |
| 36485 | 7: 19929090 | 36486 | 7: 19934553 | 36487 | 7: 19940802 | 36488 | 7: 19948369 | 36489 | 7: 19954948 |
| 36490 | 7: 19962302 | 36491 | 7: 19974241 | 36492 | 7: 19977349 | 36494 | 7: 20018689 | 36495 | 7: 20036738 |
| 36496 | 7: 20053188 | 36497 | 7: 20057534 | 36498 | 7: 20076160 | 36507 | 7: 20236317 | 36512 | 7: 20288732 |
| 36513 | 7: 20318995 | 36514 | 7: 20324208 | 36515 | 7: 20324611 | 36517 | 7: 20355414 | 36518 | 7: 20356311 |
| 36519 | 7: 20356363 | 36520 | 7: 20356383 | 36521 | 7: 20366443 | 36522 | 7: 20366466 | 36531 | 7: 20582777 |
| 36532 | 7: 20591858 | 36534 | 7: 20634478 | 36541 | 7: 20805581 | 36543 | 7: 20890886 | 36544 | 7: 20890946 |
| 36547 | 7: 20913247 | 36549 | 7: 20940338 | 36558 | 7: 21139922 | 36565 | 7: 21283821 | 36570 | 7: 21380240 |
| 36571 | 7: 21386399 | 36573 | 7: 21403549 | 36596 | 7: 21986701 | 36601 | 7: 22039959 | 36613 | 7: 22387715 |
| 36624 | 7: 22566377 | 36625 | 7: 22566448 | 36627 | 7: 22594692 | 36628 | 7: 22594727 | 36633 | 7: 22680039 |
| 36635 | 7: 22786294 | 36637 | 7: 22926260 | 36645 | 7: 23219383 | 36648 | 7: 23248102 | 36655 | 7: 23451699 |
| 36702 | 7: 24500311 | 36711 | 7: 24783223 | 36714 | 7: 24910871 | 36745 | 7: 25616363 | 36749 | 7: 25654878 |
| 36752 | 7: 25731016 | 36753 | 7: 25768089 | 36757 | 7: 25800631 | 36758 | 7: 25832593 | 36764 | 7: 25936782 |
| 36766 | 7: 26024639 | 36777 | 7: 26292161 | 36778 | 7: 26308990 | 36779 | 7: 26319916 | 36782 | 7: 26387075 |
| 36784 | 7: 26402172 | 36788 | 7: 26497485 | 36790 | 7: 26557163 | 36791 | 7: 26574444 | 36792 | 7: 26577081 |
| 36793 | 7: 26577087 | 36794 | 7: 26578715 | 36796 | 7: 26594068 | 36797 | 7: 26594073 | 36798 | 7: 26594090 |
| 36801 | 7: 26638130 | 36805 | 7: 26769987 | 36806 | 7: 26769998 | 36807 | 7: 26770016 | 36811 | 7: 26823826 |
| 36814 | 7: 26881081 | 36815 | 7: 26898868 | 36821 | 7: 27031629 | 36825 | 7: 27060226 | 36832 | 7: 27198824 |
| 36840 | 7: 27423246 | 36841 | 7: 27434826 | 36848 | 7: 27596371 | 36855 | 7: 27733288 | 36862 | 7: 27870224 |
| 36865 | 7: 28003332 | 36867 | 7: 28049997 | 36868 | 7: 28050030 | 36869 | 7: 28050091 | 36870 | 7: 28050098 |
| 36886 | 7: 28544979 | 36893 | 7: 28690143 | 36895 | 7: 28700180 | 36897 | 7: 28729276 | 36900 | 7: 28830783 |
| 36902 | 7: 28879641 | 36908 | 7: 29036081 | 36912 | 7: 29112331 | 36913 | 7: 29178982 | 36922 | 7: 29366546 |
| 36924 | 7: 29452732 | 36934 | 7: 29594179 | 36936 | 7: 29632089 | 36938 | 7: 29666350 | 36953 | 7: 29976577 |
| 36956 | 7: 30094509 | 36968 | 7: 30358105 | 36971 | 7: 30403065 | 36974 | 7: 30553468 | 36977 | 7: 30644004 |
| 36981 | 7: 30714382 | 36982 | 7: 30714446 | 36983 | 7: 30756401 | 36984 | 7: 30807818 | 36988 | 7: 30863698 |
| 36992 | 7: 30906898 | 36995 | 7: 30991586 | 36998 | 7: 31107264 | 37025 | 7: 31626205 | 37029 | 7: 31642354 |
| 37030 | 7: 31642496 | 37031 | 7: 31643684 | 37032 | 7: 31643707 | 37037 | 7: 31676529 | 37039 | 7: 31699035 |
| 37065 | 7: 32314617 | 37080 | 7: 32688342 | 37081 | 7: 32709556 | 37085 | 7: 32796768 | 37096 | 7: 33201093 |
| 37097 | 7: 33201168 | 37119 | 7: 33724498 | 37122 | 7: 33761341 | 37123 | 7: 33814744 | 37124 | 7: 33814750 |
| 37125 | 7: 33814773 | 37126 | 7: 33814812 | 37129 | 7: 33866953 | 37153 | 7: 34486136 | 37154 | 7: 34502493 |
| 37163 | 7: 34781679 | 37170 | 7: 34951198 | 37172 | 7: 34967857 | 37173 | 7: 34967898 | 37176 | 7: 35028517 |
| 37182 | 7: 35229836 | 37199 | 7: 35718668 | 37219 | 7: 36283231 | 37220 | 7: 36308740 | 37231 | 7: 36510620 |
| 37238 | 7: 36626658 | 37239 | 7: 36666622 | 37265 | 7: 37451557 | 37266 | 7: 37459216 | 37269 | 7: 37490139 |
| 37273 | 7: 37534315 | 37277 | 7: 37590371 | 37281 | 7: 37620741 | 37287 | 7: 37840929 | 37291 | 7: 37934995 |
| 37297 | 7: 38048904 | 37298 | 7: 38081521 | 37299 | 7: 38087100 | 37300 | 7: 38093801 | 37301 | 7: 38101864 |
| 37302 | 7: 38124565 | 37303 | 7: 38128610 | 37304 | 7: 38129977 | 37314 | 7: 38325007 | 37316 | 7: 38360533 |
| 37317 | 7: 38365253 | 37318 | 7: 38389751 | 37321 | 7: 38404339 | 37322 | 7: 38421353 | 37323 | 7: 38429558 |
| 37327 | 7: 38515000 | 37328 | 7: 38534271 | 37333 | 7: 38753839 | 37338 | 7: 38815021 | 37343 | 7: 38952074 |
| 37355 | 7: 39351372 | 37356 | 7: 39362501 | 37359 | 7: 39455426 | 37368 | 7: 39695685 | 37372 | 7: 39721571 |
| 37373 | 7: 39721626 | 37381 | 7: 40016354 | 37385 | 7: 40095095 | 37388 | 7: 40114076 | 37389 | 7: 40149307 |
| 37390 | 7: 40151612 | 37396 | 7: 40294613 | 37397 | 7: 40294728 | 37398 | 7: 40294729 | 37399 | 7: 40294731 |
| 37400 | 7: 40294744 | 37401 | 7: 40294745 | 37404 | 7: 40323542 | 37407 | 7: 40355854 | 37408 | 7: 40360599 |
| 37409 | 7: 40361142 | 37410 | 7: 40361184 | 37411 | 7: 40361244 | 37412 | 7: 40361336 | 37413 | 7: 40372170 |
| 37414 | 7: 40372221 | 37415 | 7: 40385848 | 37416 | 7: 40441629 | 37419 | 7: 40507729 | 37420 | 7: 40508207 |
| 37423 | 7: 40603077 | 37424 | 7: 40603099 | 37425 | 7: 40604704 | 37426 | 7: 40605637 | 37427 | 7: 40606059 |
| 37429 | 7: 40622112 | 37436 | 7: 40917929 | 37438 | 7: 40932803 | 37439 | 7: 40933182 | 37440 | 7: 40933299 |
| 37441 | 7: 40945845 | 37442 | 7: 40946121 | 37443 | 7: 40946770 | 37444 | 7: 40957931 | 37446 | 7: 40958025 |
| 37447 | 7: 40960932 | 37453 | 7: 41290614 | 37454 | 7: 41322935 | 37461 | 7: 41552605 | 37462 | 7: 41552746 |
| 37463 | 7: 41553267 | 37464 | 7: 41553442 | 37467 | 7: 41694074 | 37468 | 7: 41694108 | 37470 | 7: 41791738 |
| 37472 | 7: 41828300 | 37473 | 7: 41828310 | 37474 | 7: 41828476 | 37475 | 7: 41828489 | 37476 | 7: 41828529 |
| 37477 | 7: 41828531 | 37478 | 7: 41828618 | 37483 | 7: 42132719 | 37495 | 7: 42627355 | 37496 | 7: 42628923 |
| 37498 | 7: 42659874 | 37499 | 7: 42659928 | 37509 | 7: 43070478 | 37510 | 7: 43070495 | 37511 | 7: 43073489 |
| 37512 | 7: 43074460 | 37513 | 7: 43074485 | 37514 | 7: 43075276 | 37515 | 7: 43075327 | 37516 | 7: 43075329 |
| 37517 | 7: 43075823 | 37518 | 7: 43075911 | 37519 | 7: 43077087 | 37522 | 7: 43114270 | 37525 | 7: 43247821 |
| 37526 | 7: 43258641 | 37531 | 7: 43495660 | 37532 | 7: 43504923 | 37533 | 7: 43504924 | 37534 | 7: 43509552 |
| 37535 | 7: 43518518 | 37536 | 7: 43526310 | 37537 | 7: 43541646 | 37539 | 7: 43547038 | 37540 | 7: 43551313 |
| 37541 | 7: 43573739 | 37542 | 7: 43602807 | 37543 | 7: 43616877 | 37546 | 7: 43695498 | 37548 | 7: 43809877 |
| 37549 | 7: 43811263 | 37550 | 7: 43812894 | 37552 | 7: 43875604 | 37558 | 7: 44162642 | 37563 | 7: 44416303 |
| 37566 | 7: 44610929 | 37568 | 7: 44639553 | 37569 | 7: 44666191 | 37572 | 7: 44764045 | 37573 | 7: 44764061 |
| 37574 | 7: 44789031 | 37575 | 7: 44789067 | 37576 | 7: 44789088 | 37584 | 7: 44943643 | 37585 | 7: 45003392 |
| 37591 | 7: 45101749 | 37599 | 7: 45350646 | 37600 | 7: 45355058 | 37605 | 7: 45445789 | 37614 | 7: 45661047 |
| 37619 | 7: 45951627 | 37620 | 7: 45994439 | 37621 | 7: 45997784 | 37623 | 7: 46123501 | 37624 | 7: 46133078 |
| 37637 | 7: 46424169 | 37649 | 7: 46722010 | 37655 | 7: 46847399 | 37657 | 7: 46881748 | 37659 | 7: 46963800 |
| 37660 | 7: 46965141 | 37661 | 7: 46965986 | 37662 | 7: 46966917 | 37663 | 7: 46969143 | 37678 | 7: 47071431 |
| 37680 | 7: 47200029 | 37694 | 7: 47663425 | 37696 | 7: 47675422 | 37697 | 7: 47681261 | 37701 | 7: 47796975 |
| 37722 | 7: 48566329 | 37730 | 7: 48673881 | 37771 | 7: 50021166 | 37780 | 7: 50639584 | 37792 | 7: 51149038 |
| 37796 | 7: 51377867 | 37798 | 7: 51400935 | 37801 | 7: 51429431 | 37803 | 7: 51450812 | 37811 | 7: 51923554 |
| 37812 | 7: 52003650 | 37815 | 7: 52173201 | 37817 | 7: 52176528 | 37818 | 7: 52177126 | 37819 | 7: 52179498 |
| 37820 | 7: 52181770 | 37821 | 7: 52182473 | 37822 | 7: 52183049 | 37823 | 7: 52183519 | 37824 | 7: 52186624 |
| 37825 | 7: 52193152 | 37826 | 7: 52196823 | 37827 | 7: 52197432 | 37828 | 7: 52198757 | 37829 | 7: 52199982 |
| 37830 | 7: 52207035 | 37831 | 7: 52210371 | 37833 | 7: 52224828 | 37834 | 7: 52225521 | 37835 | 7: 52227549 |
| 37836 | 7: 52227592 | 37838 | 7: 52249785 | 37839 | 7: 52251276 | 37840 | 7: 52251297 | 37841 | 7: 52251426 |
| 37842 | 7: 52251546 | 37843 | 7: 52251618 | 37844 | 7: 52251861 | 37845 | 7: 52252987 | 37846 | 7: 52263699 |
| 37847 | 7: 52264079 | 37848 | 7: 52264430 | 37849 | 7: 52264511 | 37850 | 7: 52264761 | 37851 | 7: 52266394 |
| 37852 | 7: 52282234 | 37853 | 7: 52289509 | 37854 | 7: 52291347 | 37855 | 7: 52293091 | 37856 | 7: 52294120 |
| 37857 | 7: 52295099 | 37858 | 7: 52296408 | 37859 | 7: 52296468 | 37860 | 7: 52298384 | 37861 | 7: 52299162 |
| 37862 | 7: 52300813 | 37863 | 7: 52303764 | 37864 | 7: 52305125 | 37865 | 7: 52305218 | 37866 | 7: 52308210 |
| 37867 | 7: 52308534 | 37868 | 7: 52308687 | 37869 | 7: 52309821 | 37870 | 7: 52339995 | 37871 | 7: 52344653 |
| 37872 | 7: 52345469 | 37873 | 7: 52348265 | 37874 | 7: 52349069 | 37878 | 7: 52567577 | 37879 | 7: 52610395 |
| 37880 | 7: 52611688 | 37884 | 7: 52667909 | 37895 | 7: 52931391 | 37898 | 7: 53021130 | 3790 | 7: 53089284 |
| 37908 | 7: 53195304 | 37920 | 7: 53551894 | 37923 | 7: 53581842 | 37924 | 7: 53587134 | 37928 | 7: 53866382 |
| 37931 | 7: 53935965 | 37934 | 7: 53979466 | 37938 | 7: 54160608 | 37943 | 7: 54227894 | 37944 | 7: 54238880 |
| 37949 | 7: 54401837 | 37950 | 7: 54414044 | 37952 | 7: 54475662 | 37983 | 7: 55275367 | 37984 | 7: 55310804 |
| 37992 | 7: 55508633 | 37997 | 7: 55613829 | 38014 | 7: 56033788 | 38027 | 7: 56270767 | 38054 | 7: 57224558 |
| 38056 | 7: 57261914 | 38060 | 7: 57381076 | 38067 | 7: 57701637 | 38068 | 7: 57702653 | 38079 | 7: 57942576 |
| 38099 | 7: 58504274 | 38100 | 7: 58564407 | 38103 | 7: 58594636 | 38106 | 7: 58736129 | 38112 | 7: 58873109 |
| 38114 | 7: 58937938 | 38118 | 7: 59041978 | 38119 | 7: 59042119 | 38120 | 7: 59044072 | 38121 | 7: 59055433 |
| 38123 | 7: 59057188 | 38124 | 7: 59057293 | 38127 | 7: 59101278 | 38128 | 7: 59111622 | 38132 | 7: 59143825 |
| 38141 | 7: 59349638 | 38142 | 7: 59349648 | 38143 | 7: 59357865 | 38144 | 7: 59366049 | 38147 | 7: 59372999 |
| 38148 | 7: 59373109 | 38149 | 7: 59373145 | 38150 | 7: 59373890 | 38151 | 7: 59373911 | 38152 | 7: 59373987 |
| 38154 | 7: 59405369 | 38161 | 7: 59476011 | 38173 | 7: 59813378 | 38174 | 7: 59851233 | 38176 | 7: 59880943 |
| 38179 | 7: 59979404 | 38180 | 7: 59993171 | 38183 | 7: 60051047 | 38187 | 7: 60113080 | 38190 | 7: 60121204 |
| 38191 | 7: 60146715 | 38192 | 7: 60159491 | 38202 | 7: 60502888 | 38205 | 7: 60568272 | 38213 | 7: 60834283 |
| 38220 | 7: 60976830 | 38222 | 7: 60986666 | 38223 | 7: 60989492 | 38225 | 7: 61027791 | 38226 | 7: 61067590 |
| 38227 | 7: 61075693 | 38230 | 7: 61121888 | 38231 | 7: 61125177 | 38234 | 7: 61147314 | 38235 | 7: 61157706 |
| 38236 | 7: 61175816 | 38239 | 7: 61222597 | 38241 | 7: 61229844 | 38242 | 7: 61229883 | 38243 | 7: 61234323 |
| 38244 | 7: 61234418 | 38245 | 7: 61240972 | 38250 | 7: 61334946 | 38251 | 7: 61352203 | 38252 | 7: 61373465 |
| 38255 | 7: 61436635 | 38262 | 7: 61491892 | 38265 | 7: 61518179 | 38267 | 7: 61555353 | 38271 | 7: 61582159 |
| 38275 | 7: 61778550 | 38282 | 7: 62019992 | 38284 | 7: 62026620 | 38292 | 7: 62285057 | 38294 | 7: 62296401 |
| 38298 | 7: 62370721 | 38300 | 7: 62436131 | 38302 | 7: 62469301 | 38303 | 7: 62475081 | 38304 | 7: 62508971 |
| 38307 | 7: 62555449 | 38310 | 7: 62601203 | 38311 | 7: 62614056 | 38315 | 7: 62677859 | 38329 | 7: 62720115 |
| 38340 | 7: 62942190 | 38354 | 7: 63240676 | 38365 | 7: 63685100 | 38378 | 7: 63974136 | 38389 | 7: 64332584 |
| 38410 | 7: 64916386 | 38416 | 7: 65002376 | 38419 | 7: 65066376 | 38421 | 7: 65074340 | 38433 | 7: 65274641 |
| 38435 | 7: 65330563 | 38439 | 7: 65403716 | 38444 | 7: 65453830 | 38453 | 7: 65721041 | 38464 | 7: 65972504 |
| 38466 | 7: 65984655 | 38467 | 7: 66008306 | 38468 | 7: 66011532 | 38471 | 7: 66093673 | 38472 | 7: 66101631 |
| 38473 | 7: 66102398 | 38474 | 7: 66104176 | 38475 | 7: 66105023 | 38476 | 7: 66105843 | 38477 | 7: 66108875 |
| 38478 | 7: 66109426 | 38479 | 7: 66110363 | 38480 | 7: 66112663 | 38481 | 7: 66113133 | 38482 | 7: 66114775 |
| 38483 | 7: 66117158 | 38484 | 7: 66117914 | 38485 | 7: 66118467 | 38486 | 7: 66120071 | 38487 | 7: 66121243 |
| 38488 | 7: 66122354 | 38489 | 7: 66122876 | 38490 | 7: 66124410 | 38491 | 7: 66125761 | 38492 | 7: 66126649 |
| 38493 | 7: 66130675 | 38494 | 7: 66134907 | 38495 | 7: 66146626 | 38496 | 7: 66158927 | 38512 | 7: 66629354 |
| 38513 | 7: 66638097 | 38514 | 7: 66640996 | 38519 | 7: 66842880 | 38523 | 7: 66907257 | 38532 | 7: 67112605 |
| 38540 | 7: 67368109 | 38549 | 7: 67650949 | 38550 | 7: 67663732 | 38551 | 7: 67677225 | 38556 | 7: 67803883 |
| 38582 | 7: 68478076 | 38595 | 7: 68892260 | 38597 | 7: 68928960 | 38598 | 7: 68949630 | 38601 | 7: 68987039 |
| 38602 | 7: 68998753 | 38604 | 7: 69001013 | 38606 | 7: 69049520 | 38611 | 7: 69144296 | 38615 | 7: 69172516 |
| 38618 | 7: 69198573 | 38624 | 7: 69399622 | 38627 | 7: 69502402 | 38628 | 7: 69503500 | 38631 | 7: 69517359 |
| 38645 | 7: 69896069 | 38649 | 7: 69988788 | 38652 | 7: 70030654 | 38654 | 7: 70060807 | 38660 | 7: 70128127 |
| 38675 | 7: 70701637 | 38676 | 7: 70732259 | 38692 | 7: 71222059 | 38695 | 7: 71353347 | 38704 | 7: 71545001 |
| 38705 | 7: 71558907 | 38733 | 7: 72053452 | 38737 | 7: 72207894 | 38738 | 7: 72232062 | 38744 | 7: 72362246 |
| 38759 | 7: 72898450 | 38762 | 7: 72962658 | 38770 | 7: 73184647 | 38775 | 7: 73473535 | 38793 | 7: 73942821 |
| 38805 | 7: 74306314 | 38806 | 7: 74306327 | 38807 | 7: 74306379 | 38809 | 7: 74321645 | 38818 | 7: 74667948 |
| 38824 | 7: 74772424 | 38825 | 7: 74834470 | 38827 | 7: 74855482 | 38828 | 7: 74855556 | 38829 | 7: 74861008 |
| 38834 | 7: 74922579 | 38835 | 7: 74934509 | 38836 | 7: 74967965 | 38838 | 7: 75008858 | 38839 | 7: 75009945 |
| 38844 | 7: 75141363 | 38845 | 7: 75145201 | 38846 | 7: 75157547 | 38848 | 7: 75201764 | 38860 | 7: 75609551 |
| 38862 | 7: 75636089 | 38863 | 7: 75677642 | 38864 | 7: 75677689 | 38871 | 7: 75802119 | 38872 | 7: 75814579 |
| 38877 | 7: 75942645 | 38886 | 7: 76249772 | 38887 | 7: 76260400 | 38888 | 7: 76279595 | 38890 | 7: 76306268 |
| 38891 | 7: 76342697 | 38892 | 7: 76347497 | 38894 | 7: 76370749 | 38898 | 7: 76449574 | 38910 | 7: 76725621 |
| 38911 | 7: 76765051 | 38914 | 7: 76875833 | 38915 | 7: 76875983 | 38919 | 7: 77004376 | 38939 | 7: 77408753 |
| 38960 | 7: 77903085 | 38966 | 7: 77985310 | 38970 | 7: 78110195 | 38974 | 7: 78196572 | 38975 | 7: 78196650 |
| 38977 | 7: 78217247 | 39034 | 7: 79217925 | 39044 | 7: 79409160 | 39050 | 7: 79575037 | 39051 | 7: 79575100 |
| 39055 | 7: 79623429 | 39057 | 7: 79676699 | 39061 | 7: 79735833 | 39062 | 7: 79735898 | 39070 | 7: 79930582 |
| 39071 | 7: 79953397 | 39074 | 7: 80074984 | 39081 | 7: 80181414 | 39086 | 7: 80322587 | 39091 | 7: 80397326 |
| 39092 | 7: 80423038 | 39094 | 7: 80460624 | 39095 | 7: 80461082 | 39097 | 7: 80469480 | 39098 | 7: 80471094 |
| 39100 | 7: 80478643 | 39101 | 7: 80503605 | 39102 | 7: 80503765 | 39104 | 7: 80509969 | 39105 | 7: 80514758 |
| 39109 | 7: 80546745 | 39113 | 7: 80594054 | 39116 | 7: 80662788 | 39130 | 7: 81016584 | 39138 | 7: 81135163 |
| 39139 | 7: 81142008 | 39140 | 7: 81142055 | 39141 | 7: 81142172 | 39142 | 7: 81174306 | 39144 | 7: 81184476 |
| 39145 | 7: 81184486 | 39151 | 7: 81313732 | 39158 | 7: 81392597 | 39159 | 7: 81392623 | 39160 | 7: 81392634 |
| 39161 | 7: 81392650 | 39162 | 7: 81392653 | 39163 | 7: 81392665 | 39170 | 7: 81654928 | 39175 | 7: 81798512 |
| 39186 | 7: 82216490 | 39187 | 7: 82219959 | 39189 | 7: 82230892 | 39190 | 7: 82248295 | 39198 | 7: 82549435 |
| 39200 | 7: 82554305 | 39206 | 7: 82662900 | 39207 | 7: 82662928 | 39208 | 7: 82662929 | 39214 | 7: 82764185 |
| 39224 | 7: 83044183 | 39244 | 7: 83544032 | 39264 | 7: 84108168 | 39265 | 7: 84108203 | 39266 | 7: 84108212 |
| 39267 | 7: 84118928 | 39269 | 7: 84144485 | 39273 | 7: 84207697 | 39275 | 7: 84239228 | 39279 | 7: 84354399 |
| 39280 | 7: 84354448 | 39284 | 7: 84424155 | 39285 | 7: 84450073 | 39288 | 7: 84509794 | 39301 | 7: 85123948 |
| 39307 | 7: 85293142 | 39308 | 7: 85293161 | 39309 | 7: 85293169 | 39323 | 7: 85845513 | 39324 | 7: 85845564 |
| 39325 | 7: 85875827 | 39328 | 7: 85965693 | 39337 | 7: 86289890 | 39338 | 7: 86289929 | 39340 | 7: 86348377 |
| 39341 | 7: 86348400 | 39345 | 7: 86462459 | 39348 | 7: 86544147 | 39349 | 7: 86566118 | 39353 | 7: 86638215 |
| 39379 | 7: 87401188 | 39388 | 7: 87608805 | 39389 | 7: 87608884 | 39390 | 7: 87608885 | 39391 | 7: 87608911 |
| 39394 | 7: 87779029 | 39395 | 7: 87841745 | 39403 | 7: 88117633 | 39411 | 7: 88379769 | 39419 | 7: 88587478 |
| 39427 | 7: 88790932 | 39428 | 7: 88796649 | 39433 | 7: 88942654 | 39435 | 7: 88966573 | 39436 | 7: 89035670 |
| 39440 | 7: 89116248 | 39445 | 7: 89148646 | 39446 | 7: 89184830 | 39450 | 7: 89247931 | 39454 | 7: 89369998 |
| 39478 | 7: 90049487 | 39483 | 7: 90127121 | 39486 | 7: 90166602 | 39487 | 7: 90166627 | 39488 | 7: 90167634 |
| 39496 | 7: 90329807 | 39511 | 7: 90598533 | 39514 | 7: 90688919 | 39523 | 7: 90779825 | 39558 | 7: 90831595 |
| 39565 | 7: 90845831 | 39596 | 7: 91489178 | 39611 | 7: 91726773 | 39653 | 7: 92762601 | 39671 | 7: 93433611 |
| 39679 | 7: 93718483 | 39680 | 7: 93727310 | 39682 | 7: 93774135 | 39693 | 7: 94215835 | 39694 | 7: 94269949 |
| 39699 | 7: 94382446 | 39703 | 7: 94439368 | 39708 | 7: 94574747 | 39717 | 7: 94849950 | 39718 | 7: 94854494 |
| 39719 | 7: 94854604 | 39720 | 7: 94854627 | 39724 | 7: 94945483 | 39729 | 7: 95020011 | 39730 | 7: 95021828 |
| 39733 | 7: 95079030 | 39735 | 7: 95116563 | 39736 | 7: 95158884 | 39743 | 7: 95405216 | 39754 | 7: 95645495 |
| 39767 | 7: 95958530 | 39771 | 7: 96074523 | 39772 | 7: 96087310 | 39775 | 7: 96098877 | 39778 | 7: 96109770 |
| 39781 | 7: 96127017 | 39782 | 7: 96127023 | 39783 | 7: 96127094 | 39784 | 7: 96140233 | 39785 | 7: 96140250 |
| 39786 | 7: 96140274 | 39787 | 7: 96140286 | 39788 | 7: 96140316 | 39789 | 7: 96140325 | 39790 | 7: 96146797 |
| 39791 | 7: 96152289 | 39793 | 7: 96169102 | 39806 | 7: 96180272 | 39807 | 7: 96180277 | 39808 | 7: 96180291 |
| 39810 | 7: 96196608 | 39811 | 7: 96196642 | 39813 | 7: 96202522 | 39829 | 7: 96220995 | 39831 | 7: 96221193 |
| 39868 | 7: 96278344 | 39911 | 7: 96314599 | 39912 | 7: 96315078 | 39934 | 7: 96355415 | 39939 | 7: 96370734 |
| 39941 | 7: 96387738 | 39950 | 7: 96555097 | 39952 | 7: 96601732 | 39954 | 7: 96613640 | 39985 | 7: 97508892 |
| 39986 | 7: 97508905 | 39988 | 7: 97541930 | 39990 | 7: 97603739 | 39991 | 7: 97629287 | 39997 | 7: 97747540 |
| 40003 | 7: 97959533 | 40019 | 7: 98300666 | 40020 | 7: 98348623 | 40021 | 7: 98348636 | 40028 | 7: 98543164 |
| 40048 | 7: 99313338 | 40070 | 7: 99845218 | 40072 | 7: 99865657 | 40075 | 7: 99914902 | 40082 | 7: 100079502 |
| 40083 | 7: 100082753 | 40086 | 7: 100118655 | 40087 | 7: 100118835 | 40088 | 7: 100136909 | 40089 | 7: 100144865 |
| 40098 | 7: 100358134 | 40099 | 7: 100395807 | 40116 | 7: 100797612 | 40129 | 7: 101254706 | 40133 | 7: 101358810 |
| 40139 | 7: 101563788 | 40142 | 7: 101606446 | 40143 | 7: 101606448 | 40147 | 7: 101667972 | 40153 | 7: 101808436 |
| 40154 | 7: 101857168 | 40157 | 7: 102004868 | 40164 | 7: 102285994 | 40173 | 7: 102580748 | 40174 | 7: 102582243 |
| 40175 | 7: 102585831 | 40176 | 7: 102589674 | 40177 | 7: 102594188 | 40178 | 7: 102599028 | 40179 | 7: 102609048 |
| 40181 | 7: 102620944 | 40192 | 7: 102888318 | 40196 | 7: 102988342 | 40198 | 7: 103035483 | 40200 | 7: 103047606 |
| 40219 | 7: 103414818 | 40280 | 7: 105164324 | 40292 | 7: 105387524 | 40298 | 7: 105535552 | 40322 | 7: 106096645 |
| 40326 | 7: 106249472 | 40327 | 7: 106254713 | 40348 | 7: 106643847 | 40351 | 7: 106742504 | 40352 | 7: 106742506 |
| 40360 | 7: 106848432 | 40362 | 7: 106869396 | 40369 | 7: 107016860 | 40370 | 7: 107023813 | 40373 | 7: 107095006 |
| 40376 | 7: 107124835 | 40383 | 7: 107308750 | 40403 | 7: 108184476 | 40407 | 7: 108325718 | 40408 | 7: 108337537 |
| 40418 | 7: 108563367 | 40431 | 7: 108821941 | 40433 | 7: 108869029 | 40439 | 7: 108994618 | 40441 | 7: 109029038 |
| 40442 | 7: 109029077 | 40443 | 7: 109029102 | 40458 | 7: 109275280 | 40480 | 7: 109999314 | 40498 | 8: 373283 |
| 40506 | 8: 643950 | 40510 | 8: 764642 | 40511 | 8: 780102 | 40514 | 8: 800236 | 40515 | 8: 816422 |
| 40517 | 8: 844855 | 40520 | 8: 882426 | 40521 | 8: 883861 | 40535 | 8: 1082935 | 40536 | 8: 1086523 |
| 40548 | 8: 1362737 | 40552 | 8: 1579017 | 40564 | 8: 1757679 | 40571 | 8: 1832243 | 40579 | 8: 1939311 |
| 40594 | 8: 2317585 | 40606 | 8: 2582570 | 40616 | 8: 3008685 | 40620 | 8: 3178994 | 40621 | 8: 3219275 |
| 40630 | 8: 3400647 | 40631 | 8: 3416328 | 40632 | 8: 3416882 | 40667 | 8: 4251182 | 40668 | 8: 4273155 |
| 40669 | 8: 4274587 | 40670 | 8: 4276578 | 40671 | 8: 4280150 | 40672 | 8: 4283775 | 40685 | 8: 4639116 |
| 40687 | 8: 4644380 | 40688 | 8: 4644486 | 40690 | 8: 4708141 | 40693 | 8: 4759903 | 40698 | 8: 4908669 |
| 40707 | 8: 5249662 | 40708 | 8: 5270683 | 40709 | 8: 5270703 | 40710 | 8: 5270802 | 40711 | 8: 5270815 |
| 40712 | 8: 5332706 | 40728 | 8: 5760796 | 40735 | 8: 5869022 | 40741 | 8: 6027760 | 40765 | 8: 6756792 |
| 40766 | 8: 6764623 | 40785 | 8: 7181322 | 40786 | 8: 7181335 | 40803 | 8: 7581528 | 40805 | 8: 7596757 |
| 40806 | 8: 7598915 | 40807 | 8: 7601383 | 40808 | 8: 7603103 | 40809 | 8: 7611610 | 40810 | 8: 7618608 |
| 40811 | 8: 7619933 | 40812 | 8: 7620831 | 40813 | 8: 7625351 | 40818 | 8: 7818361 | 40826 | 8: 8001917 |
| 40832 | 8: 8175029 | 40842 | 8: 8393306 | 40844 | 8: 8477216 | 40848 | 8: 8618577 | 40857 | 8: 8769353 |
| 40866 | 8: 9042180 | 40868 | 8: 9063209 | 40869 | 8: 9119208 | 40889 | 8: 9619039 | 40890 | 8: 9619107 |
| 40891 | 8: 9619135 | 40892 | 8: 9623038 | 40893 | 8: 9627034 | 40894 | 8: 9627109 | 40895 | 8: 9627275 |
| 40896 | 8: 9627321 | 40905 | 8: 9890341 | 40906 | 8: 9901901 | 40910 | 8: 10031350 | 40917 | 8: 10339279 |
| 40925 | 8: 10604669 | 40931 | 8: 10696791 | 40934 | 8: 10712286 | 40935 | 8: 10712485 | 40936 | 8: 10712536 |
| 40939 | 8: 10783394 | 40942 | 8: 10854210 | 40943 | 8: 10854709 | 40952 | 8: 11049154 | 40955 | 8: 11128689 |
| 40980 | 8: 11615963 | 40981 | 8: 11626767 | 40983 | 8: 11655158 | 40985 | 8: 11693820 | 40990 | 8: 11853891 |
| 41001 | 8: 12089230 | 41008 | 8: 12344836 | 41012 | 8: 12433144 | 41026 | 8: 12914075 | 41036 | 8: 13348241 |
| 41038 | 8: 13393062 | 41047 | 8: 13722063 | 41097 | 8: 15148225 | 41103 | 8: 15333923 | 41109 | 8: 15473403 |
| 41113 | 8: 15497145 | 41114 | 8: 15497203 | 41115 | 8: 15497248 | 41116 | 8: 15533442 | 41120 | 8: 15590887 |
| 41121 | 8: 15590953 | 41124 | 8: 15685122 | 41128 | 8: 15765706 | 41133 | 8: 15967637 | 41143 | 8: 16338822 |
| 41145 | 8: 16399613 | 41155 | 8: 16592732 | 41158 | 8: 16699848 | 41162 | 8: 16791579 | 41163 | 8: 16792851 |
| 41171 | 8: 16926726 | 41174 | 8: 16945156 | 41178 | 8: 17032464 | 41179 | 8: 17032475 | 41180 | 8: 17032488 |
| 41182 | 8: 17048252 | 41183 | 8: 17055738 | 41184 | 8: 17055749 | 41185 | 8: 17055775 | 41186 | 8: 17056869 |
| 41187 | 8: 17061563 | 41188 | 8: 17064508 | 41193 | 8: 17304347 | 41195 | 8: 17305220 | 41196 | 8: 17307048 |
| 41197 | 8: 17307131 | 41206 | 8: 17548023 | 41231 | 8: 18280322 | 41242 | 8: 18613707 | 41246 | 8: 18768280 |
| 41257 | 8: 19052433 | 41288 | 8: 19935582 | 41301 | 8: 20342622 | 41342 | 8: 21329932 | 41361 | 8: 21984030 |
| 41363 | 8: 22082837 | 41364 | 8: 22100760 | 41365 | 8: 22113575 | 41396 | 8: 23324599 | 41401 | 8: 23427337 |
| 41421 | 8: 24057660 | 41434 | 8: 24296988 | 41440 | 8: 24394806 | 41442 | 8: 24405999 | 41444 | 8: 24443570 |
| 41450 | 8: 24643364 | 41452 | 8: 24674588 | 41454 | 8: 24687934 | 41455 | 8: 24689851 | 41456 | 8: 24692069 |
| 41457 | 8: 24700212 | 41459 | 8: 24720840 | 41462 | 8: 24752172 | 41466 | 8: 24771365 | 41467 | 8: 24786333 |
| 41468 | 8: 24793655 | 41469 | 8: 24802374 | 41470 | 8: 24812615 | 41471 | 8: 24820473 | 41472 | 8: 24827262 |
| 41475 | 8: 24874038 | 41476 | 8: 24875382 | 41477 | 8: 24877159 | 41478 | 8: 24877700 | 41479 | 8: 24878235 |
| 41480 | 8: 24878922 | 41481 | 8: 24888678 | 41482 | 8: 24891103 | 41483 | 8: 24895594 | 41485 | 8: 24933323 |
| 41486 | 8: 24938771 | 41487 | 8: 24940757 | 41488 | 8: 24955657 | 41489 | 8: 24957890 | 41490 | 8: 24961237 |
| 41491 | 8: 24966416 | 41492 | 8: 24974478 | 41493 | 8: 24979200 | 41494 | 8: 24988001 | 41495 | 8: 24992186 |
| 41496 | 8: 24996358 | 41497 | 8: 25006948 | 41498 | 8: 25013387 | 41499 | 8: 25035664 | 41500 | 8: 25041072 |
| 41501 | 8: 25059424 | 41506 | 8: 25124953 | 41507 | 8: 25126729 | 41509 | 8: 25140406 | 41510 | 8: 25144505 |
| 41511 | 8: 25146183 | 41512 | 8: 25148717 | 41513 | 8: 25150222 | 41514 | 8: 25152059 | 41515 | 8: 25154593 |
| 41516 | 8: 25157388 | 41517 | 8: 25160394 | 41518 | 8: 25165311 | 41519 | 8: 25166943 | 41520 | 8: 25169477 |
| 41522 | 8: 25174754 | 41523 | 8: 25177709 | 41524 | 8: 25180230 | 41525 | 8: 25185692 | 41526 | 8: 25188439 |
| 41527 | 8: 25198273 | 41528 | 8: 25200755 | 41530 | 8: 25214312 | 41531 | 8: 25227176 | 41532 | 8: 25233627 |
| 41533 | 8: 25235124 | 41534 | 8: 25238388 | 41535 | 8: 25240252 | 41537 | 8: 25243485 | 41538 | 8: 25254140 |
| 41539 | 8: 25257480 | 41540 | 8: 25259038 | 41541 | 8: 25261263 | 41542 | 8: 25263852 | 41543 | 8: 25265485 |
| 41544 | 8: 25266937 | 41545 | 8: 25268844 | 41546 | 8: 25274693 | 41547 | 8: 25282832 | 41548 | 8: 25294627 |
| 41557 | 8: 25487377 | 41558 | 8: 25500162 | 41561 | 8: 25551702 | 41580 | 8: 25993221 | 41584 | 8: 26067879 |
| 41602 | 8: 26560396 | 41604 | 8: 26591338 | 41614 | 8: 26825483 | 41621 | 8: 26909158 | 41631 | 8: 27313786 |
| 41633 | 8: 27383296 | 41637 | 8: 27558131 | 41649 | 8: 27894769 | 41652 | 8: 27969801 | 41654 | 8: 28067472 |
| 41664 | 8: 28411601 | 41665 | 8: 28411658 | 41670 | 8: 28576597 | 41673 | 8: 28652470 | 41678 | 8: 28903569 |
| 41679 | 8: 28935823 | 41691 | 8: 29203761 | 41702 | 8: 29452933 | 41706 | 8: 29509148 | 41707 | 8: 29543012 |
| 41728 | 8: 29972472 | 41730 | 8: 30031385 | 41734 | 8: 30076221 | 41737 | 8: 30253112 | 41750 | 8: 30514176 |
| 41754 | 8: 30666192 | 41757 | 8: 30707536 | 41764 | 8: 30964101 | 41765 | 8: 30965820 | 41767 | 8: 31027893 |
| 41769 | 8: 31049984 | 41770 | 8: 31056192 | 41773 | 8: 31128535 | 41776 | 8: 31166613 | 41780 | 8: 31290392 |
| 41781 | 8: 31300884 | 41785 | 8: 31442029 | 41786 | 8: 31453763 | 41790 | 8: 31579785 | 41793 | 8: 31643340 |
| 41801 | 8: 31867242 | 41802 | 8: 31895156 | 41859 | 8: 33917004 | 41888 | 8: 34615024 | 41894 | 8: 34800931 |
| 41895 | 8: 34800977 | 41896 | 8: 34801009 | 41919 | 8: 35445047 | 41923 | 8: 35459817 | 41936 | 8: 35833781 |
| 41938 | 8: 35859948 | 41943 | 8: 35930089 | 41953 | 8: 36065706 | 41956 | 8: 36096463 | 41958 | 8: 36187712 |
| 41962 | 8: 36233237 | 41965 | 8: 36292654 | 41968 | 8: 36343251 | 41985 | 8: 36842382 | 41986 | 8: 36851180 |
| 41995 | 8: 37031806 | 41996 | 8: 37054784 | 41998 | 8: 37095387 | 42008 | 8: 37407050 | 42010 | 8: 37435996 |
| 42023 | 8: 37797692 | 42024 | 8: 37854305 | 42028 | 8: 37928176 | 42044 | 8: 38675399 | 42047 | 8: 38691615 |
| 42056 | 8: 39019428 | 42061 | 8: 39290623 | 42067 | 8: 39583278 | 42068 | 8: 39584890 | 42069 | 8: 39621719 |
| 42074 | 8: 39696110 | 42075 | 8: 39718793 | 42077 | 8: 39768968 | 42078 | 8: 39769005 | 42079 | 8: 39809755 |
| 42081 | 8: 39864901 | 42091 | 8: 40003850 | 42101 | 8: 40171823 | 42102 | 8: 40219628 | 42103 | 8: 40219668 |
| 42105 | 8: 40257959 | 42106 | 8: 40287413 | 42107 | 8: 40306339 | 42109 | 8: 40334191 | 42110 | 8: 40334321 |
| 42114 | 8: 40410096 | 42131 | 8: 40743770 | 42135 | 8: 40830870 | 42150 | 8: 41247220 | 42152 | 8: 41309293 |
| 42165 | 8: 41611046 | 42168 | 8: 41682500 | 42169 | 8: 41693466 | 42170 | 8: 41697104 | 42174 | 8: 41759986 |
| 42176 | 8: 41790111 | 42179 | 8: 41858426 | 42180 | 8: 41890619 | 42181 | 8: 41897019 | 42199 | 8: 42436416 |
| 42211 | 8: 42709975 | 42212 | 8: 42720374 | 42216 | 8: 42880518 | 42220 | 8: 42958668 | 42221 | 8: 42958711 |
| 42222 | 8: 42958836 | 42226 | 8: 43016661 | 42236 | 8: 43367984 | 42245 | 8: 43812387 | 42246 | 8: 43815004 |
| 42250 | 8: 43957564 | 42255 | 8: 43998136 | 42256 | 8: 43998213 | 42258 | 8: 44010856 | 42269 | 8: 44431429 |
| 42273 | 8: 44588721 | 42282 | 8: 44879292 | 42283 | 8: 44895474 | 42285 | 8: 44916012 | 42295 | 8: 45238924 |
| 42301 | 8: 45399787 | 42304 | 8: 45427010 | 42306 | 8: 45447403 | 42309 | 8: 45519975 | 42310 | 8: 45544435 |
| 42314 | 8: 45672590 | 42316 | 8: 45691728 | 42321 | 8: 45748288 | 42327 | 8: 45881905 | 42328 | 8: 45949542 |
| 42333 | 8: 46053215 | 42334 | 8: 46058853 | 42347 | 8: 46184772 | 42352 | 8: 46322367 | 42358 | 8: 46514754 |
| 42362 | 8: 46662471 | 42363 | 8: 46688442 | 42367 | 8: 46794717 | 42378 | 8: 47069272 | 42379 | 8: 47096555 |
| 42383 | 8: 47242828 | 42384 | 8: 47301441 | 42386 | 8: 47301545 | 42396 | 8: 47442795 | 42397 | 8: 47454300 |
| 42398 | 8: 47468132 | 42413 | 8: 47827950 | 42416 | 8: 47959914 | 42443 | 8: 48870474 | 42452 | 8: 48980291 |
| 42455 | 8: 49060228 | 42456 | 8: 49060267 | 42457 | 8: 49060352 | 42458 | 8: 49071203 | 42459 | 8: 49082742 |
| 42465 | 8: 49227434 | 42466 | 8: 49248066 | 42470 | 8: 49305124 | 42471 | 8: 49328971 | 42472 | 8: 49332342 |
| 42484 | 8: 49820121 | 42490 | 8: 50120835 | 42493 | 8: 50158365 | 42496 | 8: 50210739 | 42502 | 8: 50343730 |
| 42505 | 8: 50410404 | 42526 | 8: 51083850 | 42529 | 8: 51149379 | 42530 | 8: 51154605 | 42537 | 8: 51409152 |
| 42549 | 8: 51645019 | 42553 | 8: 51697635 | 42563 | 8: 51983681 | 42569 | 8: 52152405 | 42570 | 8: 52157895 |
| 42573 | 8: 52201603 | 42584 | 8: 52496347 | 42593 | 8: 52683991 | 42597 | 8: 52775082 | 42606 | 8: 53099576 |
| 42607 | 8: 53099649 | 42608 | 8: 53099684 | 42618 | 8: 53465873 | 42622 | 8: 53559314 | 42626 | 8: 53663149 |
| 42628 | 8: 53760217 | 42643 | 8: 54223148 | 42650 | 8: 54393899 | 42663 | 8: 54661982 | 42665 | 8: 54767805 |
| 42666 | 8: 54767855 | 42676 | 8: 54981068 | 42677 | 8: 54981074 | 42678 | 8: 54981156 | 42693 | 8: 55456387 |
| 42697 | 8: 55552471 | 42699 | 8: 55552575 | 42707 | 8: 55861711 | 42708 | 8: 55861771 | 42709 | 8: 55869592 |
| 42711 | 8: 55889240 | 42718 | 8: 56082094 | 42719 | 8: 56082109 | 42723 | 8: 56355997 | 42732 | 8: 56476308 |
| 42736 | 8: 56567532 | 42749 | 8: 56898409 | 42752 | 8: 56940553 | 42755 | 8: 57067743 | 42756 | 8: 57083802 |
| 42757 | 8: 57097646 | 42769 | 8: 57431848 | 42770 | 8: 57436089 | 42771 | 8: 57441513 | 42774 | 8: 57491864 |
| 42777 | 8: 57503925 | 42781 | 8: 57561608 | 42784 | 8: 57609698 | 42785 | 8: 57624471 | 42794 | 8: 58010694 |
| 42796 | 8: 58065599 | 42799 | 8: 58159003 | 42803 | 8: 58281685 | 42817 | 8: 58837870 | 42827 | 8: 59185961 |
| 42831 | 8: 59304332 | 42833 | 8: 59343826 | 42843 | 8: 59578341 | 42845 | 8: 59585372 | 42846 | 8: 59591203 |
| 42847 | 8: 59595742 | 42853 | 8: 59747078 | 42856 | 8: 59839831 | 42861 | 8: 59908902 | 42867 | 8: 59979908 |
| 42869 | 8: 59997834 | 42873 | 8: 60218203 | 42874 | 8: 60232849 | 42875 | 8: 60252660 | 42877 | 8: 60376601 |
| 42878 | 8: 60387912 | 42880 | 8: 60393687 | 42881 | 8: 60407147 | 42882 | 8: 60408080 | 42883 | 8: 60413868 |
| 42885 | 8: 60422237 | 42887 | 8: 60434309 | 42888 | 8: 60445473 | 42893 | 8: 60483449 | 42901 | 8: 60773154 |
| 42906 | 8: 60847978 | 42928 | 8: 61569513 | 42931 | 8: 61676872 | 42953 | 8: 62298007 | 42955 | 8: 62361352 |
| 42956 | 8: 62370401 | 42957 | 8: 62370423 | 42960 | 8: 62476415 | 42961 | 8: 62476489 | 42962 | 8: 62476593 |
| 42963 | 8: 62481551 | 42964 | 8: 62481563 | 42965 | 8: 62492081 | 42966 | 8: 62492171 | 42967 | 8: 62492207 |
| 42969 | 8: 62554727 | 42970 | 8: 62556949 | 42977 | 8: 62779236 | 42980 | 8: 62823371 | 42985 | 8: 62978731 |
| 42986 | 8: 62978820 | 42989 | 8: 63076318 | 42991 | 8: 63182958 | 42998 | 8: 63428149 | 43008 | 8: 63577043 |
| 43025 | 8: 64001632 | 43026 | 8: 64003121 | 43031 | 8: 64111218 | 43038 | 8: 64443505 | 43041 | 8: 64480556 |
| 43042 | 8: 64512625 | 43050 | 8: 64890322 | 43059 | 8: 65128134 | 43060 | 8: 65147458 | 43063 | 8: 65217066 |
| 43064 | 8: 65219496 | 43065 | 8: 65219567 | 43073 | 8: 65486798 | 43074 | 8: 65486879 | 43075 | 8: 65486964 |
| 43076 | 8: 65487030 | 43077 | 8: 65487045 | 43078 | 8: 65487167 | 43079 | 8: 65487520 | 43080 | 8: 65506614 |
| 43092 | 8: 66005380 | 43094 | 8: 66041697 | 43095 | 8: 66071027 | 43096 | 8: 66071753 | 43099 | 8: 66095067 |
| 43100 | 8: 66095107 | 43102 | 8: 66110177 | 43103 | 8: 66110963 | 43104 | 8: 66111005 | 43117 | 8: 66467235 |
| 43129 | 8: 66780866 | 43130 | 8: 66781633 | 43132 | 8: 66784915 | 43134 | 8: 66833433 | 43135 | 8: 66833492 |
| 43139 | 8: 66987197 | 43140 | 8: 66999877 | 43141 | 8: 67000007 | 43143 | 8: 67013017 | 43146 | 8: 67072138 |
| 43148 | 8: 67105385 | 43149 | 8: 67105408 | 43150 | 8: 67121705 | 43153 | 8: 67145134 | 43154 | 8: 67154528 |
| 43158 | 8: 67392786 | 43162 | 8: 67566051 | 43164 | 8: 67576272 | 43166 | 8: 67607555 | 43167 | 8: 67613943 |
| 43170 | 8: 67704512 | 43171 | 8: 67731230 | 43182 | 8: 67844754 | 43183 | 8: 67844773 | 43200 | 8: 68399674 |
| 43216 | 8: 68844096 | 43217 | 8: 68866548 | 43218 | 8: 68866614 | 43223 | 8: 68960840 | 43224 | 8: 69009944 |
| 43227 | 8: 69053764 | 43236 | 8: 69156564 | 43237 | 8: 69156580 | 43239 | 8: 69252951 | 43241 | 8: 69325956 |
| 43242 | 8: 69421607 | 43258 | 8: 69882196 | 43261 | 8: 69947806 | 43262 | 8: 69974129 | 43263 | 8: 70014064 |
| 43264 | 8: 70029487 | 43265 | 8: 70039617 | 43280 | 8: 70405010 | 43286 | 8: 70466364 | 43288 | 8: 70482064 |
| 43289 | 8: 70489717 | 43290 | 8: 70492869 | 43292 | 8: 70501163 | 43297 | 8: 70592634 | 43303 | 8: 70815570 |
| 43306 | 8: 70893052 | 43310 | 8: 70958608 | 43319 | 8: 71238004 | 43336 | 8: 71880675 | 43360 | 8: 72468833 |
| 43361 | 8: 72468916 | 43362 | 8: 72515403 | 43364 | 8: 72551579 | 43366 | 8: 72558376 | 43367 | 8: 72561704 |
| 43372 | 8: 72825901 | 43373 | 8: 72828377 | 43375 | 8: 72837275 | 43376 | 8: 72844608 | 43377 | 8: 72845970 |
| 43378 | 8: 72904078 | 43380 | 8: 72930313 | 43404 | 8: 73494061 | 43406 | 8: 73515301 | 43412 | 8: 73656442 |
| 43423 | 8: 73915262 | 43432 | 8: 74168959 | 43436 | 8: 74325878 | 43440 | 8: 74465417 | 43444 | 8: 74553402 |
| 43445 | 8: 74558074 | 43446 | 8: 74558704 | 43452 | 8: 74712678 | 43458 | 8: 74817911 | 43462 | 8: 74863602 |
| 43463 | 8: 74883099 | 43467 | 8: 75123238 | 43471 | 8: 75268745 | 43477 | 8: 75477646 | 43481 | 8: 75562796 |
| 43484 | 8: 75700129 | 43487 | 8: 75740079 | 43490 | 8: 75853292 | 43497 | 8: 76175098 | 43507 | 8: 76433813 |
| 43508 | 8: 76434785 | 43509 | 8: 76434815 | 43511 | 8: 76464537 | 43513 | 8: 76492709 | 43515 | 8: 76515741 |
| 43521 | 8: 76664342 | 43522 | 8: 76681835 | 43523 | 8: 76684305 | 43524 | 8: 76687450 | 43525 | 8: 76703859 |
| 43526 | 8: 76709352 | 43529 | 8: 76742010 | 43530 | 8: 76752971 | 43531 | 8: 76754631 | 43532 | 8: 76760562 |
| 43533 | 8: 76762856 | 43534 | 8: 76764812 | 43535 | 8: 76768935 | 43538 | 8: 76828964 | 43539 | 8: 76835585 |
| 43540 | 8: 76839168 | 43541 | 8: 76841937 | 43542 | 8: 76843765 | 43543 | 8: 76844513 | 43544 | 8: 76846849 |
| 43545 | 8: 76850281 | 43546 | 8: 76854581 | 43547 | 8: 76870958 | 43548 | 8: 76873029 | 43551 | 8: 76975704 |
| 43553 | 8: 76993039 | 43554 | 8: 77006329 | 43556 | 8: 77136545 | 43564 | 8: 77562434 | 43581 | 8: 78166070 |
| 43589 | 8: 78358551 | 43595 | 8: 78556780 | 43616 | 8: 79447931 | 43642 | 8: 80268872 | 43648 | 8: 80405770 |
| 43652 | 8: 80565130 | 43656 | 8: 80613713 | 43660 | 8: 80836273 | 43669 | 8: 80974113 | 43686 | 8: 81278385 |
| 43692 | 8: 81389738 | 43695 | 8: 81454715 | 43696 | 8: 81454750 | 43698 | 8: 81487284 | 43699 | 8: 81487295 |
| 43704 | 8: 81616321 | 43712 | 8: 81744964 | 43716 | 8: 81860516 | 43727 | 8: 82113238 | 43728 | 8: 82116405 |
| 43730 | 8: 82160329 | 43751 | 8: 82676827 | 43752 | 8: 82677581 | 43753 | 8: 82677988 | 43759 | 8: 82823172 |
| 43762 | 8: 82865703 | 43769 | 8: 83085781 | 43782 | 8: 83515046 | 43790 | 8: 83738651 | 43794 | 8: 83793151 |
| 43795 | 8: 83793154 | 43796 | 8: 83794591 | 43797 | 8: 83794760 | 43799 | 8: 83842360 | 43803 | 8: 83879316 |
| 43808 | 8: 83976437 | 43809 | 8: 84045184 | 43811 | 8: 84100704 | 43812 | 8: 84123211 | 43813 | 8: 84134245 |
| 43815 | 8: 84172945 | 43829 | 8: 84494416 | 43835 | 8: 84811346 | 43842 | 8: 84912804 | 43858 | 8: 85366261 |
| 43873 | 8: 85725330 | 43875 | 8: 85804392 | 43884 | 8: 85987078 | 43888 | 8: 86079018 | 43891 | 8: 86115895 |
| 43892 | 8: 86133024 | 43895 | 8: 86219410 | 43896 | 8: 86226370 | 43905 | 8: 86537816 | 43912 | 8: 86719772 |
| 43914 | 8: 86756176 | 43916 | 8: 86779557 | 43917 | 8: 86793653 | 43920 | 8: 86935675 | 43921 | 8: 86961975 |
| 43949 | 8: 87523043 | 43955 | 8: 87701857 | 43967 | 8: 88033337 | 43968 | 8: 88126576 | 43977 | 8: 88363375 |
| 43986 | 8: 88562919 | 43987 | 8: 88572133 | 43995 | 8: 88712829 | 43997 | 8: 88762685 | 44001 | 8: 88829394 |
| 44007 | 8: 88990195 | 44013 | 8: 89098414 | 44014 | 8: 89113184 | 44016 | 8: 89140453 | 44018 | 8: 89141946 |
| 44019 | 8: 89141971 | 44020 | 8: 89142000 | 44021 | 8: 89142063 | 44024 | 8: 89269473 | 44033 | 8: 89533107 |
| 44034 | 8: 89533116 | 44035 | 8: 89533121 | 44037 | 8: 89557638 | 44041 | 8: 89571402 | 44046 | 8: 89722190 |
| 44049 | 8: 89770693 | 44056 | 8: 89926318 | 44059 | 8: 90070089 | 44062 | 8: 90193893 | 44084 | 8: 90853473 |
| 44085 | 8: 90853557 | 44086 | 8: 90853588 | 44087 | 8: 90853599 | 44101 | 8: 91152034 | 44102 | 8: 91161810 |
| 44106 | 8: 91289222 | 44110 | 8: 91381933 | 44111 | 8: 91382574 | 44129 | 8: 91948504 | 44137 | 8: 91989858 |
| 44138 | 8: 91995185 | 44143 | 8: 92008855 | 44149 | 8: 92136613 | 44168 | 8: 92747513 | 44178 | 8: 93068614 |
| 44179 | 8: 93068725 | 44204 | 8: 93841029 | 44208 | 8: 94082045 | 44212 | 8: 94260204 | 44220 | 8: 94620469 |
| 44221 | 8: 94620534 | 44222 | 8: 94621015 | 44223 | 8: 94623988 | 44224 | 8: 94624900 | 44225 | 8: 94672379 |
| 44226 | 8: 94690978 | 44232 | 8: 94939320 | 44241 | 8: 95337173 | 44259 | 8: 95796796 | 44263 | 8: 95951526 |
| 44264 | 8: 95977921 | 44266 | 8: 96012156 | 44272 | 8: 96165982 | 44273 | 8: 96193360 | 44281 | 8: 96384779 |
| 44282 | 8: 96445578 | 44289 | 8: 96573262 | 44308 | 8: 97157793 | 44317 | 8: 97299078 | 44319 | 8: 97368811 |
| 44326 | 8: 97495202 | 44327 | 8: 97557161 | 44329 | 8: 97577094 | 44330 | 8: 97601680 | 44335 | 8: 97795768 |
| 44350 | 8: 98245729 | 44360 | 8: 98592583 | 44362 | 8: 98625126 | 44367 | 8: 98741152 | 44368 | 8: 98785042 |
| 44370 | 8: 98829420 | 44375 | 8: 98919680 | 44386 | 8: 99142994 | 44392 | 8: 99207386 | 44397 | 8: 99291886 |
| 44400 | 8: 99333533 | 44404 | 8: 99388416 | 44405 | 8: 99406177 | 44407 | 8: 99412670 | 44409 | 8: 99476683 |
| 44413 | 8: 99525142 | 44415 | 8: 99551509 | 44425 | 8: 99654305 | 44429 | 8: 99703737 | 44435 | 8: 99959755 |
| 44436 | 8: 99982505 | 44437 | 8: 100010762 | 44446 | 8: 100374163 | 44448 | 8: 100401670 | 44449 | 8: 100409615 |
| 44453 | 8: 100428898 | 44455 | 8: 100470224 | 44466 | 8: 100654637 | 44471 | 8: 100832272 | 44472 | 8: 100876059 |
| 44478 | 8: 101026237 | 44482 | 8: 101247151 | 44485 | 8: 101301768 | 44486 | 8: 101312150 | 44494 | 8: 101441924 |
| 44496 | 8: 101485459 | 44500 | 8: 101643451 | 44502 | 8: 101701776 | 44506 | 8: 101779830 | 44507 | 8: 101800844 |
| 44517 | 8: 102113941 | 44523 | 8: 102234774 | 44525 | 8: 102290809 | 44526 | 8: 102291109 | 44527 | 8: 102291157 |
| 44528 | 8: 102291228 | 44529 | 8: 102291230 | 44530 | 8: 102291256 | 44531 | 8: 102291263 | 44532 | 8: 102293933 |
| 44533 | 8: 102293974 | 44534 | 8: 102299598 | 44535 | 8: 102299601 | 44536 | 8: 102305536 | 44538 | 8: 102377736 |
| 44540 | 8: 102402762 | 44541 | 8: 102408863 | 44542 | 8: 102411405 | 44546 | 8: 102434252 | 44554 | 8: 102594527 |
| 44555 | 8: 102594773 | 44556 | 8: 102595590 | 44557 | 8: 102617174 | 44558 | 8: 102618622 | 44560 | 8: 102626997 |
| 44561 | 8: 102634985 | 44562 | 8: 102640018 | 44564 | 8: 102642339 | 44566 | 8: 102645680 | 44567 | 8: 102648250 |
| 44568 | 8: 102648741 | 44577 | 8: 102834472 | 44579 | 8: 102889304 | 44583 | 8: 102931846 | 44596 | 8: 103307658 |
| 44597 | 8: 103316620 | 44598 | 8: 103323065 | 44599 | 8: 103323527 | 44600 | 8: 103415237 | 44601 | 8: 103442278 |
| 44602 | 8: 103449094 | 44604 | 8: 103484976 | 44605 | 8: 103489848 | 44606 | 8: 103500362 | 44607 | 8: 103516850 |
| 44608 | 8: 103529815 | 44609 | 8: 103560463 | 44610 | 8: 103572704 | 44611 | 8: 103575282 | 44612 | 8: 103580891 |
| 44613 | 8: 103584228 | 44614 | 8: 103588272 | 44615 | 8: 103589059 | 44616 | 8: 103591535 | 44618 | 8: 103596979 |
| 44619 | 8: 103598675 | 44620 | 8: 103603170 | 44621 | 8: 103612096 | 44622 | 8: 103612794 | 44624 | 8: 103623951 |
| 44625 | 8: 103634947 | 44626 | 8: 103640204 | 44627 | 8: 103645354 | 44628 | 8: 103649615 | 44630 | 8: 103673828 |
| 44634 | 8: 103783688 | 44646 | 8: 104113096 | 44647 | 8: 104114568 | 44650 | 8: 104204807 | 44654 | 8: 104251576 |
| 44655 | 8: 104271837 | 44658 | 8: 104292759 | 44659 | 8: 104322730 | 44661 | 8: 104414493 | 44663 | 8: 104420746 |
| 44669 | 8: 104546178 | 44674 | 8: 104630353 | 44679 | 8: 104760159 | 44681 | 8: 104815425 | 44694 | 8: 105160429 |
| 44716 | 8: 105525844 | 44719 | 8: 105592649 | 44722 | 8: 105630176 | 44725 | 8: 105696501 | 44727 | 8: 105765759 |
| 44728 | 8: 105774110 | 44732 | 8: 105846896 | 44739 | 8: 106082143 | 44744 | 8: 106427938 | 44759 | 8: 106936156 |
| 44760 | 8: 106961448 | 44761 | 8: 106981761 | 44762 | 8: 107001526 | 44808 | 8: 108127783 | 44829 | 8: 108978247 |
| 44850 | 8: 109853668 | 44855 | 8: 110002325 | 44857 | 8: 110059433 | 44860 | 8: 110157715 | 44862 | 8: 110244165 |
| 44863 | 8: 110244337 | 44865 | 8: 110248495 | 44866 | 8: 110251397 | 44870 | 8: 110445148 | 44871 | 8: 110445202 |
| 44872 | 8: 110445222 | 44874 | 8: 110540868 | 44880 | 8: 110749028 | 44882 | 8: 110764849 | 44888 | 8: 110972844 |
| 44890 | 8: 110979223 | 44892 | 8: 111002543 | 44893 | 8: 111081452 | 44897 | 8: 111284112 | 44932 | 9: 349617 |
| 44935 | 9: 449265 | 44965 | 9: 761293 | 44966 | 9: 761398 | 44970 | 9: 946489 | 44983 | 9: 1228475 |
| 44984 | 9: 1228520 | 44985 | 9: 1228587 | 44986 | 9: 1228611 | 44987 | 9: 1228669 | 44997 | 9: 1530507 |
| 44998 | 9: 1530528 | 45013 | 9: 1926540 | 45016 | 9: 2022731 | 45017 | 9: 2022740 | 45084 | 9: 4060756 |
| 45090 | 9: 4309847 | 45101 | 9: 4645222 | 45115 | 9: 5086559 | 45159 | 9: 6390476 | 45161 | 9: 6450647 |
| 45163 | 9: 6559884 | 45174 | 9: 6887577 | 45189 | 9: 7542350 | 45206 | 9: 7865211 | 45219 | 9: 8218235 |
| 45220 | 9: 8218462 | 45223 | 9: 8257979 | 45224 | 9: 8258011 | 45225 | 9: 8258047 | 45241 | 9: 8662565 |
| 45242 | 9: 8691938 | 45245 | 9: 8740852 | 45246 | 9: 8740922 | 45247 | 9: 8741004 | 45255 | 9: 9002672 |
| 45259 | 9: 9073133 | 45265 | 9: 9212931 | 45269 | 9: 9363423 | 45281 | 9: 9866377 | 45288 | 9: 10026119 |
| 45290 | 9: 10179658 | 45293 | 9: 10229582 | 45295 | 9: 10270107 | 45302 | 9: 10376631 | 45307 | 9: 10412914 |
| 45343 | 9: 10650283 | 45377 | 9: 10813074 | 45393 | 9: 11379384 | 45398 | 9: 11488885 | 45413 | 9: 11816817 |
| 45418 | 9: 11933227 | 45428 | 9: 12091110 | 45431 | 9: 12153363 | 45433 | 9: 12204845 | 45435 | 9: 12250871 |
| 45445 | 9: 12499635 | 45446 | 9: 12509673 | 45452 | 9: 12645170 | 45471 | 9: 12766564 | 45481 | 9: 13100266 |
| 45485 | 9: 13105495 | 45491 | 9: 13132484 | 45496 | 9: 13287682 | 45498 | 9: 13361022 | 45503 | 9: 13494908 |
| 45509 | 9: 13684115 | 45510 | 9: 13698663 | 45517 | 9: 13880641 | 45520 | 9: 13989518 | 45523 | 9: 14095633 |
| 45524 | 9: 14124920 | 45527 | 9: 14203195 | 45540 | 9: 14468437 | 45545 | 9: 14564006 | 45548 | 9: 14665440 |
| 45550 | 9: 14717693 | 45564 | 9: 15013572 | 45568 | 9: 15038319 | 45570 | 9: 15071350 | 45571 | 9: 15081586 |
| 45574 | 9: 15123580 | 45577 | 9: 15163893 | 45578 | 9: 15175648 | 45585 | 9: 15369931 | 45587 | 9: 15406354 |
| 45595 | 9: 15497571 | 45596 | 9: 15499363 | 45597 | 9: 15499412 | 45598 | 9: 15499535 | 45599 | 9: 15511160 |
| 45602 | 9: 15527205 | 45604 | 9: 15548110 | 45605 | 9: 15553535 | 45606 | 9: 15558085 | 45607 | 9: 15560500 |
| 45608 | 9: 15560527 | 45609 | 9: 15562142 | 45623 | 9: 15687216 | 45631 | 9: 15868930 | 45644 | 9: 16138159 |
| 45653 | 9: 16384844 | 45657 | 9: 16537269 | 45674 | 9: 17031734 | 45675 | 9: 17031771 | 45687 | 9: 17461271 |
| 45692 | 9: 17595872 | 45717 | 9: 18430308 | 45721 | 9: 18529694 | 45722 | 9: 18552564 | 45724 | 9: 18645348 |
| 45726 | 9: 18717619 | 45727 | 9: 18731789 | 45729 | 9: 18755368 | 45731 | 9: 18791756 | 45732 | 9: 18791819 |
| 45733 | 9: 18791889 | 45738 | 9: 18936509 | 45743 | 9: 19046499 | 45745 | 9: 19139676 | 45753 | 9: 19435053 |
| 45776 | 9: 20235228 | 45779 | 9: 20303916 | 45816 | 9: 21552246 | 45824 | 9: 21653355 | 45833 | 9: 21833987 |
| 45835 | 9: 21852388 | 45840 | 9: 21940817 | 45846 | 9: 22080892 | 45848 | 9: 22133199 | 45865 | 9: 22573520 |
| 45867 | 9: 22594113 | 45874 | 9: 22795139 | 45879 | 9: 22958243 | 45889 | 9: 23151477 | 45890 | 9: 23173931 |
| 45894 | 9: 23218165 | 45898 | 9: 23339249 | 45900 | 9: 23430616 | 45901 | 9: 23441779 | 45904 | 9: 23456305 |
| 45906 | 9: 23472649 | 45909 | 9: 23546774 | 45915 | 9: 23672358 | 45925 | 9: 23857068 | 45932 | 9: 24029631 |
| 45937 | 9: 24145793 | 45943 | 9: 24238183 | 45951 | 9: 24426538 | 45970 | 9: 25002484 | 45980 | 9: 25350079 |
| 45981 | 9: 25353185 | 45989 | 9: 25530018 | 45997 | 9: 25755576 | 45999 | 9: 25785154 | 46003 | 9: 25878075 |
| 46010 | 9: 26028894 | 46015 | 9: 26134547 | 46018 | 9: 26208349 | 46028 | 9: 26467397 | 46039 | 9: 26838528 |
| 46040 | 9: 26868103 | 46041 | 9: 26898919 | 46048 | 9: 27183994 | 46054 | 9: 27249330 | 46057 | 9: 27304567 |
| 46058 | 9: 27317016 | 46061 | 9: 27408248 | 46071 | 9: 27587922 | 46077 | 9: 27702582 | 46094 | 9: 28136433 |
| 46108 | 9: 28431328 | 46121 | 9: 28644696 | 46127 | 9: 28779206 | 46165 | 9: 29391430 | 46177 | 9: 29530654 |
| 46179 | 9: 29544266 | 46183 | 9: 29615614 | 46205 | 9: 30230309 | 46207 | 9: 30256998 | 46208 | 9: 30257023 |
| 46209 | 9: 30257052 | 46212 | 9: 30289673 | 46219 | 9: 30469930 | 46224 | 9: 30615123 | 46227 | 9: 30674435 |
| 46229 | 9: 30739336 | 46237 | 9: 30892065 | 46239 | 9: 30932509 | 46240 | 9: 30932519 | 46241 | 9: 30932556 |
| 46247 | 9: 31029451 | 46249 | 9: 31141266 | 46252 | 9: 31227971 | 46253 | 9: 31227973 | 46255 | 9: 31253066 |
| 46261 | 9: 31394461 | 46271 | 9: 31555978 | 46272 | 9: 31588762 | 46273 | 9: 31588794 | 46275 | 9: 31725734 |
| 46283 | 9: 31984894 | 46290 | 9: 32052617 | 46291 | 9: 32063888 | 46292 | 9: 32063963 | 46296 | 9: 32225638 |
| 46300 | 9: 32284534 | 46301 | 9: 32291884 | 46307 | 9: 32437975 | 46308 | 9: 32501496 | 46312 | 9: 32638914 |
| 46315 | 9: 32720871 | 46342 | 9: 33372243 | 46349 | 9: 33488593 | 46350 | 9: 33496695 | 46356 | 9: 33730807 |
| 46357 | 9: 33736686 | 46358 | 9: 33736719 | 46365 | 9: 33922999 | 46376 | 9: 34234529 | 46377 | 9: 34239640 |
| 46383 | 9: 34398858 | 46405 | 9: 35059112 | 46422 | 9: 35505083 | 46424 | 9: 35630351 | 46428 | 9: 35723297 |
| 46429 | 9: 35723312 | 46430 | 9: 35723316 | 46431 | 9: 35723347 | 46432 | 9: 35723412 | 46438 | 9: 35923167 |
| 46445 | 9: 36108895 | 46446 | 9: 36118700 | 46448 | 9: 36196365 | 46457 | 9: 36321163 | 46464 | 9: 36515253 |
| 46466 | 9: 36617540 | 46468 | 9: 36686271 | 46470 | 9: 36765440 | 46475 | 9: 36837540 | 46482 | 9: 36952973 |
| 46485 | 9: 36987967 | 46495 | 9: 37162141 | 46500 | 9: 37248499 | 46501 | 9: 37262739 | 46504 | 9: 37446666 |
| 46518 | 9: 37786775 | 46532 | 9: 38042996 | 46538 | 9: 38200604 | 46540 | 9: 38214307 | 46541 | 9: 38222757 |
| 46542 | 9: 38234690 | 46548 | 9: 38345134 | 46549 | 9: 38346604 | 46550 | 9: 38349255 | 46551 | 9: 38361398 |
| 46555 | 9: 38439726 | 46556 | 9: 38458789 | 46558 | 9: 38513565 | 46560 | 9: 38544090 | 46562 | 9: 38561388 |
| 46568 | 9: 38630128 | 46575 | 9: 38725892 | 46578 | 9: 38828299 | 46585 | 9: 38890408 | 46589 | 9: 38943133 |
| 46590 | 9: 38963419 | 46603 | 9: 39377711 | 46610 | 9: 39610211 | 46611 | 9: 39633208 | 46612 | 9: 39637416 |
| 46613 | 9: 39638258 | 46614 | 9: 39639117 | 46616 | 9: 39648286 | 46622 | 9: 39764057 | 46623 | 9: 39784094 |
| 46624 | 9: 39793093 | 46627 | 9: 39807347 | 46628 | 9: 39807953 | 46629 | 9: 39811555 | 46634 | 9: 39934012 |
| 46635 | 9: 39956055 | 46638 | 9: 40007646 | 46646 | 9: 40343063 | 46647 | 9: 40346754 | 46649 | 9: 40351241 |
| 46650 | 9: 40353887 | 46651 | 9: 40359510 | 46652 | 9: 40394895 | 46653 | 9: 40405455 | 46658 | 9: 40559500 |
| 46664 | 9: 40689518 | 46670 | 9: 40792949 | 46673 | 9: 40849838 | 46674 | 9: 40851485 | 46675 | 9: 40876197 |
| 46676 | 9: 40920502 | 46677 | 9: 40928750 | 46678 | 9: 40936292 | 46679 | 9: 40949947 | 46680 | 9: 40960994 |
| 46681 | 9: 40986746 | 46684 | 9: 41057481 | 46690 | 9: 41209197 | 46698 | 9: 41519871 | 46702 | 9: 41630405 |
| 46705 | 9: 41713976 | 46712 | 9: 42008068 | 46714 | 9: 42049316 | 46715 | 9: 42066260 | 46716 | 9: 42073188 |
| 46721 | 9: 42199260 | 46722 | 9: 42202691 | 46723 | 9: 42206745 | 46724 | 9: 42207524 | 46726 | 9: 42218456 |
| 46738 | 9: 42539807 | 46741 | 9: 42738988 | 46742 | 9: 42751569 | 46743 | 9: 42754079 | 46751 | 9: 43079312 |
| 46752 | 9: 43096509 | 46754 | 9: 43117290 | 46755 | 9: 43117292 | 46756 | 9: 43117313 | 46757 | 9: 43117336 |
| 46758 | 9: 43117351 | 46759 | 9: 43155466 | 46760 | 9: 43155519 | 46761 | 9: 43155650 | 46766 | 9: 43352867 |
| 46779 | 9: 43685269 | 46787 | 9: 43948226 | 46803 | 9: 44341413 | 46817 | 9: 44599779 | 46820 | 9: 44637804 |
| 46826 | 9: 44854910 | 46842 | 9: 45505888 | 46845 | 9: 45550200 | 46868 | 9: 46443428 | 46877 | 9: 46667396 |
| 46883 | 9: 46847687 | 46895 | 9: 47304057 | 46923 | 9: 48075628 | 46930 | 9: 48336145 | 46939 | 9: 48787417 |
| 46954 | 9: 49224046 | 46955 | 9: 49225376 | 46982 | 9: 49910883 | 46995 | 9: 50277864 | 47007 | 9: 50567297 |
| 47008 | 9: 50567298 | 47009 | 9: 50567390 | 47012 | 9: 50646985 | 47015 | 9: 50694324 | 47019 | 9: 50797460 |
| 47059 | 9: 52046890 | 47080 | 9: 52645687 | 47088 | 9: 52787927 | 47093 | 9: 52848517 | 47095 | 9: 52861826 |
| 47120 | 9: 53314350 | 47139 | 9: 54071333 | 47143 | 9: 54193058 | 47146 | 9: 54279421 | 47153 | 9: 54522488 |
| 47161 | 9: 54675346 | 47181 | 9: 55237255 | 47192 | 9: 55526568 | 47193 | 9: 55526660 | 47194 | 9: 55529308 |
| 47228 | 9: 56645296 | 47230 | 9: 56687693 | 47234 | 9: 56760440 | 47236 | 9: 56787195 | 47245 | 9: 57057433 |
| 47259 | 9: 57574987 | 47260 | 9: 57574995 | 47262 | 9: 57634613 | 47272 | 9: 57793857 | 47276 | 9: 57849001 |
| 47284 | 9: 58149441 | 47287 | 9: 58311080 | 47288 | 9: 58333810 | 47293 | 9: 58457036 | 47301 | 9: 58652669 |
| 47314 | 9: 59159045 | 47351 | 9: 59715217 | 47353 | 9: 59771120 | 47355 | 9: 59857183 | 47362 | 9: 60072130 |
| 47364 | 9: 60134972 | 47367 | 9: 60247427 | 47369 | 9: 60291706 | 47372 | 9: 60358510 | 47377 | 9: 60481368 |
| 47380 | 9: 60536321 | 47383 | 9: 60631920 | 47388 | 9: 60745480 | 47393 | 9: 60896389 | 47394 | 9: 60903838 |
| 47397 | 9: 60928250 | 47398 | 9: 60928272 | 47400 | 9: 60929060 | 47401 | 9: 60930496 | 47402 | 9: 60932205 |
| 47403 | 9: 60936701 | 47406 | 9: 61034023 | 47420 | 9: 61625663 | 47428 | 9: 61883689 | 47437 | 9: 62153095 |
| 47438 | 9: 62160605 | 47441 | 9: 62239934 | 47444 | 9: 62407115 | 47446 | 9: 62441325 | 47448 | 9: 62541808 |
| 47449 | 9: 62541844 | 47450 | 9: 62541925 | 47451 | 9: 62569285 | 47453 | 9: 62596000 | 47475 | 9: 63213406 |
| 47481 | 9: 63334601 | 47482 | 9: 63334709 | 47483 | 9: 63334737 | 47488 | 9: 63564229 | 47490 | 9: 63662723 |
| 47495 | 9: 63879901 | 47505 | 9: 64262650 | 47522 | 9: 64653298 | 47523 | 9: 64675425 | 47526 | 9: 64806879 |
| 47527 | 9: 64818979 | 47536 | 9: 65014222 | 47537 | 9: 65014886 | 47538 | 9: 65015900 | 47539 | 9: 65016625 |
| 47540 | 9: 65017299 | 47541 | 9: 65017970 | 47542 | 9: 65018705 | 47543 | 9: 65020386 | 47544 | 9: 65022012 |
| 47545 | 9: 65022960 | 47546 | 9: 65024531 | 47547 | 9: 65025971 | 47548 | 9: 65026593 | 47549 | 9: 65027537 |
| 47550 | 9: 65029266 | 47551 | 9: 65031641 | 47552 | 9: 65033856 | 47553 | 9: 65034695 | 47554 | 9: 65037061 |
| 47555 | 9: 65038847 | 47556 | 9: 65039895 | 47557 | 9: 65041060 | 47558 | 9: 65041925 | 47559 | 9: 65046945 |
| 47560 | 9: 65048753 | 47561 | 9: 65051331 | 47562 | 9: 65052345 | 47564 | 9: 65062111 | 47565 | 9: 65063669 |
| 47566 | 9: 65065713 | 47567 | 9: 65069167 | 47568 | 9: 65072452 | 47569 | 9: 65074602 | 47570 | 9: 65075729 |
| 47571 | 9: 65076561 | 47572 | 9: 65077195 | 47573 | 9: 65077758 | 47574 | 9: 65078630 | 47575 | 9: 65118677 |
| 47576 | 9: 65123323 | 47578 | 9: 65179118 | 47579 | 9: 65235666 | 47580 | 9: 65243193 | 47581 | 9: 65244259 |
| 47586 | 9: 65383024 | 47589 | 9: 65443065 | 47590 | 9: 65446545 | 47591 | 9: 65478206 | 47592 | 9: 65483403 |
| 47598 | 9: 65568211 | 47599 | 9: 65570457 | 47600 | 9: 65572272 | 47602 | 9: 65604455 | 47609 | 9: 65891235 |
| 47612 | 9: 65971970 | 47615 | 9: 66011528 | 47638 | 9: 66597468 | 47648 | 9: 66989120 | 47657 | 9: 67225760 |
| 47663 | 9: 67332887 | 47668 | 9: 67459980 | 47681 | 9: 67796936 | 47682 | 9: 67823147 | 47686 | 9: 67954748 |
| 47705 | 9: 68370513 | 47708 | 9: 68427995 | 47720 | 9: 68912666 | 47721 | 9: 68917528 | 47729 | 9: 69060023 |
| 47730 | 9: 69093647 | 47731 | 9: 69104702 | 47732 | 9: 69114502 | 47738 | 9: 69297159 | 47741 | 9: 69407676 |
| 47750 | 9: 69583761 | 47751 | 9: 69599853 | 47761 | 9: 69896845 | 47767 | 9: 70012144 | 47777 | 9: 70295013 |
| 47779 | 9: 70342326 | 47780 | 9: 70347483 | 47786 | 9: 70409386 | 47787 | 9: 70409422 | 47789 | 9: 70429923 |
| 47797 | 9: 70751662 | 47798 | 9: 70753720 | 47805 | 9: 70887910 | 47809 | 9: 71049805 | 47811 | 9: 71060333 |
| 47813 | 9: 71107122 | 47815 | 9: 71116611 | 47820 | 9: 71251152 | 47822 | 9: 71292789 | 47824 | 9: 71344053 |
| 47825 | 9: 71356859 | 47826 | 9: 71384334 | 47828 | 9: 71407368 | 47829 | 9: 71445600 | 47831 | 9: 71492315 |
| 47834 | 9: 71512303 | 47835 | 9: 71513737 | 47840 | 9: 71612772 | 47844 | 9: 71651985 | 47848 | 9: 71736589 |
| 47849 | 9: 71761625 | 47864 | 9: 72304039 | 47867 | 9: 72384960 | 47877 | 9: 72575284 | 47882 | 9: 72722658 |
| 47883 | 9: 72725124 | 47891 | 9: 72888193 | 47892 | 9: 72973276 | 47893 | 9: 72989037 | 47895 | 9: 73138567 |
| 47897 | 9: 73175040 | 47902 | 9: 73223596 | 47903 | 9: 73244841 | 47915 | 9: 73594271 | 47920 | 9: 73701336 |
| 47921 | 9: 73701377 | 47937 | 9: 74031324 | 47939 | 9: 74043724 | 47948 | 9: 74315428 | 47955 | 9: 74575135 |
| 47957 | 9: 74617923 | 47959 | 9: 74622543 | 47974 | 9: 75094490 | 47975 | 9: 75114725 | 47979 | 9: 75261135 |
| 47984 | 9: 75482552 | 47987 | 9: 75505924 | 47991 | 9: 75564777 | 47995 | 9: 75627446 | 47999 | 9: 75748546 |
| 48000 | 9: 75749661 | 48001 | 9: 75754806 | 48002 | 9: 75754987 | 48014 | 9: 76061643 | 48016 | 9: 76103605 |
| 48022 | 9: 76314846 | 48023 | 9: 76319841 | 48038 | 9: 76900400 | 48041 | 9: 76954502 | 48042 | 9: 76961448 |
| 48045 | 9: 76972318 | 48048 | 9: 77037622 | 48049 | 9: 77047736 | 48050 | 9: 77049274 | 48058 | 9: 77255407 |
| 48063 | 9: 77401724 | 48071 | 9: 77497521 | 48150 | 9: 78018701 | 48157 | 9: 78383301 | 48163 | 9: 78618960 |
| 48166 | 9: 78653185 | 48173 | 9: 78856788 | 48176 | 9: 78984268 | 48192 | 9: 79629564 | 48204 | 9: 79900857 |
| 48206 | 9: 79927447 | 48209 | 9: 79992577 | 48211 | 9: 79999363 | 48220 | 9: 80143039 | 48222 | 9: 80223447 |
| 48226 | 9: 80281892 | 48235 | 9: 80379126 | 48251 | 9: 80913419 | 48252 | 9: 80926042 | 48253 | 9: 80927048 |
| 48254 | 9: 80927101 | 48255 | 9: 80935101 | 48256 | 9: 80938115 | 48260 | 9: 80960455 | 48281 | 9: 81389583 |
| 48286 | 9: 81502463 | 48289 | 9: 81518292 | 48299 | 9: 81745339 | 48304 | 9: 81841021 | 48317 | 9: 82097477 |
| 48318 | 9: 82097512 | 48319 | 9: 82100313 | 48337 | 9: 82534749 | 48362 | 9: 82854874 | 48364 | 9: 82923714 |
| 48369 | 9: 83119086 | 48374 | 9: 83237503 | 48377 | 9: 83309469 | 48391 | 9: 83641203 | 48392 | 9: 83672779 |
| 48399 | 9: 83841026 | 48400 | 9: 83857767 | 48418 | 9: 84276446 | 48447 | 9: 85144864 | 48452 | 9: 85201593 |
| 48455 | 9: 85222343 | 48457 | 9: 85277103 | 48474 | 9: 85565902 | 48476 | 9: 85582831 | 48477 | 9: 85582845 |
| 48478 | 9: 85582856 | 48479 | 9: 85582930 | 48485 | 9: 85757204 | 48486 | 9: 85777430 | 48487 | 9: 85777435 |
| 48488 | 9: 85777468 | 48489 | 9: 85777555 | 48492 | 9: 85801137 | 48494 | 9: 85812059 | 48500 | 9: 85926846 |
| 48507 | 9: 86110491 | 48514 | 9: 86329047 | 48532 | 9: 86823737 | 48534 | 9: 86935158 | 48537 | 9: 86963116 |
| 48539 | 9: 86989548 | 48541 | 9: 87028872 | 48556 | 9: 87348985 | 48561 | 9: 87384465 | 48568 | 9: 87494785 |
| 48570 | 9: 87503445 | 48571 | 9: 87504577 | 48572 | 9: 87542588 | 48573 | 9: 87544854 | 48593 | 9: 87878916 |
| 48595 | 9: 87890519 | 48596 | 9: 87917511 | 48598 | 9: 87929924 | 48599 | 9: 87952953 | 48600 | 9: 87962089 |
| 48601 | 9: 87962734 | 48603 | 9: 87966017 | 48604 | 9: 87966917 | 48605 | 9: 87975738 | 48610 | 9: 88087724 |
| 48612 | 9: 88140632 | 48613 | 9: 88160188 | 48626 | 9: 88569166 | 48628 | 9: 88636603 | 48629 | 9: 88636618 |
| 48630 | 9: 88636634 | 48635 | 9: 88732430 | 48644 | 9: 88925002 | 48646 | 9: 88943609 | 48654 | 9: 89094468 |
| 48656 | 9: 89166354 | 48658 | 9: 89189099 | 48666 | 9: 89326613 | 48667 | 9: 89345202 | 48668 | 9: 89346130 |
| 48670 | 9: 89356829 | 48671 | 9: 89373227 | 48672 | 9: 89380245 | 48673 | 9: 89387102 | 48674 | 9: 89399914 |
| 48676 | 9: 89410907 | 48677 | 9: 89417136 | 48678 | 9: 89423178 | 48680 | 9: 89436296 | 48681 | 9: 89443866 |
| 48682 | 9: 89445782 | 48683 | 9: 89445926 | 48684 | 9: 89447805 | 48685 | 9: 89454999 | 48688 | 9: 89471837 |
| 48690 | 9: 89496573 | 48698 | 9: 89664367 | 48699 | 9: 89708244 | 48700 | 9: 89708807 | 48709 | 9: 89919288 |
| 48733 | 9: 90540018 | 48744 | 9: 90795244 | 48745 | 9: 90796860 | 48750 | 9: 90844111 | 48751 | 9: 90845595 |
| 48752 | 9: 90845718 | 48762 | 9: 91108815 | 48767 | 9: 91144245 | 48776 | 9: 91423091 | 48783 | 9: 91591725 |
| 48784 | 9: 91611257 | 48786 | 9: 91670541 | 48789 | 9: 91728675 | 48790 | 9: 91734111 | 48794 | 9: 91799932 |
| 48795 | 9: 91808084 | 48802 | 9: 92017600 | 48804 | 9: 92051007 | 48808 | 9: 92146096 | 48809 | 9: 92173136 |
| 48823 | 9: 92434720 | 48827 | 9: 92618757 | 48831 | 9: 92719541 | 48833 | 9: 92726807 | 48834 | 9: 92726908 |
| 48835 | 9: 92732978 | 48844 | 9: 92935922 | 48848 | 9: 93101440 | 48860 | 9: 93255745 | 48864 | 9: 93329746 |
| 48865 | 9: 93335995 | 48868 | 9: 93434039 | 48869 | 9: 93439818 | 48871 | 9: 93544670 | 48872 | 9: 93562913 |
| 48873 | 9: 93583179 | 48874 | 9: 93589365 | 48876 | 9: 93593753 | 48878 | 9: 93648481 | 48881 | 9: 93756818 |
| 48882 | 9: 93761851 | 48888 | 9: 93864628 | 48899 | 9: 94037282 | 48902 | 9: 94059439 | 48903 | 9: 94075786 |
| 48908 | 9: 94239134 | 48909 | 9: 94242901 | 48916 | 9: 94279436 | 48925 | 9: 94506574 | 48926 | 9: 94511166 |
| 48928 | 9: 94549002 | 48929 | 9: 94552504 | 48930 | 9: 94571641 | 48938 | 9: 94770144 | 48943 | 9: 94849176 |
| 48951 | 9: 95011385 | 48952 | 9: 95011440 | 48954 | 9: 95075297 | 48957 | 9: 95117095 | 48958 | 9: 95119909 |
| 48959 | 9: 95120912 | 48960 | 9: 95120918 | 48961 | 9: 95121102 | 48963 | 9: 95137543 | 48965 | 9: 95146949 |
| 48966 | 9: 95149713 | 48967 | 9: 95151479 | 48968 | 9: 95151880 | 48969 | 9: 95154305 | 48970 | 9: 95159375 |
| 48971 | 9: 95171473 | 48972 | 9: 95239467 | 48975 | 9: 95257977 | 48996 | 9: 95960164 | 48997 | 9: 95961292 |
| 48998 | 9: 95964766 | 48999 | 9: 95965222 | 49013 | 9: 96262623 | 49016 | 9: 96335077 | 49018 | 9: 96352203 |
| 49020 | 9: 96364316 | 49021 | 9: 96365825 | 49023 | 9: 96394133 | 49026 | 9: 96454587 | 49027 | 9: 96459992 |
| 49028 | 9: 96460776 | 49029 | 9: 96462068 | 49030 | 9: 96464377 | 49031 | 9: 96464974 | 49032 | 9: 96465729 |
| 49033 | 9: 96467828 | 49034 | 9: 96471984 | 49035 | 9: 96474365 | 49036 | 9: 96475139 | 49037 | 9: 96477053 |
| 49038 | 9: 96478790 | 49039 | 9: 96480951 | 49040 | 9: 96482190 | 49041 | 9: 96487368 | 49045 | 9: 96575319 |
| 49047 | 9: 96602946 | 49050 | 9: 96636351 | 49064 | 9: 96811911 | 49067 | 9: 96862976 | 49068 | 9: 96865435 |
| 49071 | 9: 96875852 | 49072 | 9: 96886282 | 49078 | 9: 97002454 | 49079 | 9: 97005408 | 49080 | 9: 97006623 |
| 49107 | 9: 97443586 | 49108 | 9: 97443633 | 49112 | 9: 97554130 | 49114 | 9: 97571949 | 49117 | 9: 97636004 |
| 49122 | 9: 97688394 | 49125 | 9: 97734208 | 49128 | 9: 97781409 | 49129 | 9: 97814093 | 49132 | 9: 97896936 |
| 49140 | 9: 97952202 | 49142 | 9: 97987774 | 49146 | 9: 98072257 | 49147 | 9: 98090254 | 49149 | 9: 98186273 |
| 49152 | 9: 98271044 | 49162 | 9: 98520046 | 49169 | 9: 98643289 | 49173 | 9: 98706036 | 49174 | 9: 98706126 |
| 49177 | 9: 98800653 | 49185 | 9: 99049511 | 49197 | 9: 99258284 | 49201 | 9: 99320226 | 49202 | 9: 99340641 |
| 49203 | 9: 99363193 | 49205 | 9: 99375752 | 49213 | 9: 99564669 | 49223 | 9: 99707846 | 49233 | 9: 99913842 |
| 49238 | 9: 100020368 | 49239 | 9: 100037134 | 49248 | 9: 100222700 | 49254 | 9: 100288205 | 49264 | 9: 100469286 |
| 49278 | 9: 100659300 | 49294 | 9: 101011242 | 49295 | 9: 101027872 | 49296 | 9: 101037743 | 49299 | 9: 101107041 |
| 49302 | 9: 101154740 | 49305 | 9: 101206979 | 49311 | 9: 101309902 | 49313 | 9: 101329946 | 49314 | 9: 101388778 |
| 49315 | 9: 101404988 | 49320 | 9: 101473236 | 49325 | 9: 101520399 | 49330 | 9: 101680647 | 49341 | 9: 101799308 |
| 49351 | 9: 101941022 | 49353 | 9: 101969310 | 49354 | 9: 101969389 | 49355 | 9: 101970659 | 49356 | 9: 101970691 |
| 49357 | 9: 101971384 | 49358 | 9: 101971424 | 49359 | 9: 101976339 | 49360 | 9: 101977590 | 49361 | 9: 101977632 |
| 49362 | 9: 101977719 | 49363 | 9: 101977742 | 49364 | 9: 101984019 | 49365 | 9: 101984050 | 49366 | 9: 101984250 |
| 49367 | 9: 102004205 | 49368 | 9: 102034495 | 49369 | 9: 102034523 | 49370 | 9: 102034580 | 49371 | 9: 102034582 |
| 49373 | 9: 102053353 | 49375 | 9: 102065589 | 49376 | 9: 102121235 | 49377 | 9: 102127863 | 49380 | 9: 102237624 |
| 49390 | 9: 102348030 | 49391 | 9: 102364770 | 49400 | 9: 102589137 | 49402 | 9: 102618152 | 49403 | 9: 102619914 |
| 49404 | 9: 102620509 | 49405 | 9: 102622882 | 49409 | 9: 102654533 | 49414 | 9: 102687424 | 49416 | 9: 102706741 |
| 49417 | 9: 102710336 | 49422 | 9: 102795356 | 49426 | 9: 102898617 | 49427 | 9: 102898674 | 49438 | 9: 103153314 |
| 49439 | 9: 103215814 | 49446 | 9: 103329162 | 49447 | 9: 103333336 | 49448 | 9: 103336781 | 49457 | 9: 103493999 |
| 49459 | 9: 103575109 | 49460 | 9: 103582734 | 49473 | 9: 103926483 | 49474 | 9: 103926928 | 49475 | 9: 103927077 |
| 49476 | 9: 103969118 | 49477 | 9: 104003359 | 49485 | 9: 104113993 | 49489 | 9: 104355196 | 49490 | 9: 104355303 |
| 49491 | 9: 104356588 | 49492 | 9: 104356685 | 49494 | 9: 104376710 | 49501 | 10: 84875 | 49519 | 10: 695399 |
| 49526 | 10: 769669 | 49544 | 10: 1257872 | 49554 | 10: 1412883 | 49570 | 10: 1805121 | 49574 | 10: 1814057 |
| 49576 | 10: 1856119 | 49578 | 10: 1881286 | 49579 | 10: 1881323 | 49593 | 10: 2147400 | 49602 | 10: 2351254 |
| 49605 | 10: 2373566 | 49626 | 10: 2903794 | 49627 | 10: 2903827 | 49629 | 10: 2929612 | 49630 | 10: 2932512 |
| 49647 | 10: 3209297 | 49674 | 10: 3744198 | 49675 | 10: 3744214 | 49677 | 10: 3772515 | 49678 | 10: 3774094 |
| 49679 | 10: 3802315 | 49691 | 10: 4074788 | 49692 | 10: 4104638 | 49705 | 10: 4448386 | 49707 | 10: 4468373 |
| 49709 | 10: 4484450 | 49711 | 10: 4554748 | 49716 | 10: 4604295 | 49720 | 10: 4638128 | 49726 | 10: 4828321 |
| 49727 | 10: 4829589 | 49732 | 10: 5012847 | 49735 | 10: 5111561 | 49755 | 10: 5476055 | 49758 | 10: 5506479 |
| 49775 | 10: 5547565 | 49776 | 10: 5550036 | 49777 | 10: 5557416 | 49781 | 10: 5609083 | 49797 | 10: 6019241 |
| 49799 | 10: 6059501 | 49802 | 10: 6133685 | 49812 | 10: 6396765 | 49816 | 10: 6454997 | 49818 | 10: 6473524 |
| 49820 | 10: 6505409 | 49823 | 10: 6590721 | 49826 | 10: 6677707 | 49857 | 10: 7516833 | 49859 | 10: 7543493 |
| 49862 | 10: 7600499 | 49873 | 10: 7761962 | 49880 | 10: 7901116 | 49884 | 10: 8008379 | 49898 | 10: 8326560 |
| 49905 | 10: 8450920 | 49907 | 10: 8478523 | 49911 | 10: 8510644 | 49912 | 10: 8510646 | 49914 | 10: 8553549 |
| 49915 | 10: 8553909 | 49920 | 10: 8708761 | 49922 | 10: 8777935 | 49926 | 10: 8877008 | 49931 | 10: 9028658 |
| 49934 | 10: 9110701 | 49955 | 10: 9671703 | 49956 | 10: 9680242 | 49958 | 10: 9697568 | 49963 | 10: 9786271 |
| 49968 | 10: 9804614 | 49979 | 10: 10147987 | 49980 | 10: 10172215 | 49983 | 10: 10207865 | 49985 | 10: 10220078 |
| 49990 | 10: 10286771 | 49992 | 10: 10393696 | 49993 | 10: 10400749 | 49997 | 10: 10519536 | 49999 | 10: 10536820 |
| 50000 | 10: 10537343 | 50001 | 10: 10611527 | 50002 | 10: 10633817 | 50005 | 10: 10700036 | 50006 | 10: 10706615 |
| 50007 | 10: 10740773 | 50008 | 10: 10740870 | 50009 | 10: 10740872 | 50010 | 10: 10742093 | 50012 | 10: 10745459 |
| 50020 | 10: 10855774 | 50025 | 10: 10952491 | 50026 | 10: 10983147 | 50028 | 10: 10992296 | 50031 | 10: 11037910 |
| 50037 | 10: 11124874 | 50041 | 10: 11194365 | 50042 | 10: 11199580 | 50043 | 10: 11214627 | 50044 | 10: 11214653 |
| 50051 | 10: 11459762 | 50052 | 10: 11480188 | 50053 | 10: 11484963 | 50054 | 10: 11485435 | 50055 | 10: 11533676 |
| 50062 | 10: 11746501 | 50071 | 10: 11984525 | 50072 | 10: 11993432 | 50075 | 10: 12037788 | 50077 | 10: 12049463 |
| 50078 | 10: 12049470 | 50080 | 10: 12056720 | 50081 | 10: 12058675 | 50082 | 10: 12060333 | 50085 | 10: 12193255 |
| 50087 | 10: 12210976 | 50092 | 10: 12363067 | 50096 | 10: 12446219 | 50104 | 10: 12563184 | 50108 | 10: 12662259 |
| 50112 | 10: 12776928 | 50118 | 10: 13028042 | 50125 | 10: 13148280 | 50129 | 10: 13195695 | 50130 | 10: 13201885 |
| 50137 | 10: 13233050 | 50141 | 10: 13251806 | 50146 | 10: 13290512 | 50155 | 10: 13433297 | 50159 | 10: 13500606 |
| 50160 | 10: 13521494 | 50163 | 10: 13603249 | 50164 | 10: 13604834 | 50184 | 10: 13904093 | 50186 | 10: 13947394 |
| 50187 | 10: 13947449 | 50188 | 10: 13953167 | 50191 | 10: 14062089 | 50201 | 10: 14318815 | 50203 | 10: 14387176 |
| 50204 | 10: 14387540 | 50216 | 10: 14685085 | 50230 | 10: 15045027 | 50231 | 10: 15055444 | 50239 | 10: 15212747 |
| 50241 | 10: 15253352 | 50242 | 10: 15253401 | 50244 | 10: 15306720 | 50250 | 10: 15382216 | 50251 | 10: 15382286 |
| 50254 | 10: 15491085 | 50261 | 10: 15626947 | 50268 | 10: 15765399 | 50269 | 10: 15766293 | 50270 | 10: 15766923 |
| 50271 | 10: 15768048 | 50272 | 10: 15777260 | 50274 | 10: 15872387 | 50275 | 10: 15872397 | 50276 | 10: 15878690 |
| 50277 | 10: 15887011 | 50278 | 10: 15887086 | 50279 | 10: 15888077 | 50280 | 10: 15888157 | 50286 | 10: 15986933 |
| 50287 | 10: 15988001 | 50293 | 10: 16160207 | 50294 | 10: 16160289 | 50295 | 10: 16160311 | 50300 | 10: 16292572 |
| 50303 | 10: 16351611 | 50309 | 10: 16477102 | 50311 | 10: 16505051 | 50315 | 10: 16579579 | 50317 | 10: 16615178 |
| 50318 | 10: 16616715 | 50319 | 10: 16622209 | 50321 | 10: 16657010 | 50323 | 10: 16689144 | 50325 | 10: 16708640 |
| 50329 | 10: 16760750 | 50330 | 10: 16783563 | 50332 | 10: 16840169 | 50333 | 10: 16840192 | 50334 | 10: 16840220 |
| 50346 | 10: 17026997 | 50358 | 10: 17327263 | 50362 | 10: 17375623 | 50364 | 10: 17405609 | 50372 | 10: 17576175 |
| 50380 | 10: 17746328 | 50393 | 10: 18053005 | 50409 | 10: 18204972 | 50413 | 10: 18326097 | 50417 | 10: 18459800 |
| 50429 | 10: 18655176 | 50430 | 10: 18656419 | 50440 | 10: 19055444 | 50441 | 10: 19055478 | 50444 | 10: 19108089 |
| 50445 | 10: 19108125 | 50446 | 10: 19108146 | 50447 | 10: 19108188 | 50448 | 10: 19108578 | 50449 | 10: 19108623 |
| 50450 | 10: 19108629 | 50463 | 10: 19296017 | 50473 | 10: 19582204 | 50483 | 10: 19757013 | 50484 | 10: 19757066 |
| 50485 | 10: 19757109 | 50492 | 10: 19887391 | 50493 | 10: 19887438 | 50494 | 10: 19887463 | 50495 | 10: 19887464 |
| 50496 | 10: 19887506 | 50497 | 10: 19906833 | 50502 | 10: 20032774 | 50522 | 10: 20511602 | 50523 | 10: 20517856 |
| 50531 | 10: 20716836 | 50533 | 10: 20746648 | 50534 | 10: 20749216 | 50535 | 10: 20765182 | 50538 | 10: 20798013 |
| 50539 | 10: 20800521 | 50540 | 10: 20823031 | 50542 | 10: 20851467 | 50543 | 10: 20854079 | 50545 | 10: 20866367 |
| 50546 | 10: 20866872 | 50547 | 10: 20878828 | 50548 | 10: 20887914 | 50549 | 10: 20905201 | 50550 | 10: 20905351 |
| 50551 | 10: 20906045 | 50552 | 10: 20922221 | 50555 | 10: 20962445 | 50556 | 10: 20969179 | 50557 | 10: 20971568 |
| 50558 | 10: 20980278 | 50559 | 10: 20992105 | 50561 | 10: 21041241 | 50564 | 10: 21086459 | 50570 | 10: 21193718 |
| 50581 | 10: 21392700 | 50586 | 10: 21482874 | 50587 | 10: 21494322 | 50588 | 10: 21496846 | 50590 | 10: 21512555 |
| 50602 | 10: 21928460 | 50604 | 10: 21943044 | 50606 | 10: 21954776 | 50607 | 10: 21955205 | 50608 | 10: 21961681 |
| 50609 | 10: 21969630 | 50610 | 10: 21969644 | 50611 | 10: 21969862 | 50612 | 10: 21970634 | 50613 | 10: 21972554 |
| 50620 | 10: 22271591 | 50629 | 10: 22389791 | 50640 | 10: 22796412 | 50644 | 10: 23008980 | 50645 | 10: 23058608 |
| 50685 | 10: 25836315 | 50687 | 10: 25842537 | 50689 | 10: 25851554 | 50707 | 10: 26370686 | 50712 | 10: 26442221 |
| 50716 | 10: 26521248 | 50719 | 10: 26552273 | 50720 | 10: 26552368 | 50725 | 10: 26660705 | 50726 | 10: 26664869 |
| 50728 | 10: 26699741 | 50733 | 10: 26735536 | 50739 | 10: 26806421 | 50744 | 10: 26852657 | 50747 | 10: 26914900 |
| 50749 | 10: 26936311 | 50751 | 10: 26971279 | 50752 | 10: 26979102 | 50753 | 10: 26990817 | 50755 | 10: 27045849 |
| 50756 | 10: 27045893 | 50759 | 10: 27104985 | 50760 | 10: 27110316 | 50761 | 10: 27110398 | 50767 | 10: 27219754 |
| 50768 | 10: 27219761 | 50770 | 10: 27257251 | 50773 | 10: 27354015 | 50774 | 10: 27354260 | 50782 | 10: 27629698 |
| 50786 | 10: 27668710 | 50787 | 10: 27668712 | 50788 | 10: 27668749 | 50805 | 10: 28336043 | 50818 | 10: 28717710 |
| 50822 | 10: 28777408 | 50823 | 10: 28777482 | 50824 | 10: 28777483 | 50825 | 10: 28777517 | 50826 | 10: 28816832 |
| 50828 | 10: 28830145 | 50829 | 10: 28830820 | 50830 | 10: 28831951 | 50831 | 10: 28837592 | 50834 | 10: 28870874 |
| 50837 | 10: 28920327 | 50838 | 10: 28921627 | 50839 | 10: 28922441 | 50841 | 10: 28924088 | 50842 | 10: 28924117 |
| 50846 | 10: 28936523 | 50847 | 10: 28936611 | 50849 | 10: 28939012 | 50851 | 10: 28941456 | 50852 | 10: 28941564 |
| 50853 | 10: 28941645 | 50854 | 10: 28967860 | 50855 | 10: 28967993 | 50865 | 10: 29192994 | 50867 | 10: 29207986 |
| 50875 | 10: 29370574 | 50877 | 10: 29394079 | 50879 | 10: 29415078 | 50880 | 10: 29434816 | 50883 | 10: 29489538 |
| 50887 | 10: 29551880 | 50894 | 10: 29683783 | 50908 | 10: 30087181 | 50918 | 10: 30456072 | 50927 | 10: 30882706 |
| 50929 | 10: 31026079 | 50943 | 10: 31383428 | 50944 | 10: 31395880 | 50946 | 10: 31421937 | 50948 | 10: 31465645 |
| 50950 | 10: 31539600 | 50965 | 10: 31989230 | 50975 | 10: 32311663 | 50987 | 10: 32581894 | 50990 | 10: 32646966 |
| 50993 | 10: 32695364 | 50996 | 10: 32766762 | 51016 | 10: 33233733 | 51023 | 10: 33473275 | 51025 | 10: 33520438 |
| 51038 | 10: 33775393 | 51040 | 10: 33792624 | 51041 | 10: 33803176 | 51052 | 10: 34100470 | 51054 | 10: 34214726 |
| 51056 | 10: 34306045 | 51058 | 10: 34339538 | 51062 | 10: 34390466 | 51063 | 10: 34399556 | 51065 | 10: 34436191 |
| 51066 | 10: 34441644 | 51067 | 10: 34463264 | 51070 | 10: 34502888 | 51078 | 10: 34709456 | 51079 | 10: 34711929 |
| 51084 | 10: 34865113 | 51088 | 10: 34924089 | 51101 | 10: 35135295 | 51104 | 10: 35165655 | 51105 | 10: 35167815 |
| 51107 | 10: 35180569 | 51109 | 10: 35184337 | 51111 | 10: 35210635 | 51112 | 10: 35210945 | 51113 | 10: 35213913 |
| 51114 | 10: 35216558 | 51117 | 10: 35370763 | 51128 | 10: 35779938 | 51129 | 10: 35856231 | 51131 | 10: 35906682 |
| 51132 | 10: 35916585 | 51135 | 10: 35975249 | 51137 | 10: 35982115 | 51143 | 10: 36077032 | 51144 | 10: 36131193 |
| 51151 | 10: 36412382 | 51153 | 10: 36441443 | 51154 | 10: 36441686 | 51166 | 10: 36836677 | 51170 | 10: 36919070 |
| 51172 | 10: 36945666 | 51173 | 10: 36951207 | 51185 | 10: 37239881 | 51188 | 10: 37341433 | 51198 | 10: 37638488 |
| 51212 | 10: 37805187 | 51214 | 10: 37904587 | 51218 | 10: 37944601 | 51229 | 10: 38333207 | 51231 | 10: 38393603 |
| 51236 | 10: 38488614 | 51245 | 10: 38701360 | 51246 | 10: 38753237 | 51267 | 10: 39292295 | 51269 | 10: 39363047 |
| 51271 | 10: 39364510 | 51273 | 10: 39377386 | 51303 | 10: 40330913 | 51306 | 10: 40373055 | 51307 | 10: 40382252 |
| 51308 | 10: 40389854 | 51309 | 10: 40408498 | 51314 | 10: 40556907 | 51321 | 10: 40717249 | 51360 | 10: 41919935 |
| 51369 | 10: 42252004 | 51393 | 10: 42819454 | 51397 | 10: 42948816 | 51406 | 10: 43236868 | 51413 | 10: 43488771 |
| 51425 | 10: 43985352 | 51432 | 10: 44080128 | 51437 | 10: 44252723 | 51446 | 10: 44448699 | 51451 | 10: 44583693 |
| 51456 | 10: 44658796 | 51457 | 10: 44687920 | 51470 | 10: 44966040 | 51472 | 10: 45022368 | 51476 | 10: 45222091 |
| 51482 | 10: 45334197 | 51483 | 10: 45334223 | 51484 | 10: 45334295 | 51485 | 10: 45383083 | 51486 | 10: 45385249 |
| 51487 | 10: 45407393 | 51488 | 10: 45421821 | 51492 | 10: 45548786 | 51502 | 10: 45782999 | 51504 | 10: 45794472 |
| 51505 | 10: 45797020 | 51507 | 10: 45802351 | 51509 | 10: 45807766 | 51510 | 10: 45825756 | 51511 | 10: 45825757 |
| 51512 | 10: 45825760 | 51513 | 10: 45852804 | 51515 | 10: 45888410 | 51517 | 10: 45962441 | 51535 | 10: 46450572 |
| 51552 | 10: 46711744 | 51556 | 10: 46771999 | 51565 | 10: 47008189 | 51572 | 10: 47133365 | 51574 | 10: 47141503 |
| 51576 | 10: 47158682 | 51580 | 10: 47240491 | 51585 | 10: 47306453 | 51589 | 10: 47384963 | 51590 | 10: 47384967 |
| 51598 | 10: 47600619 | 51601 | 10: 47645531 | 51605 | 10: 47674190 | 51608 | 10: 47753050 | 51614 | 10: 48040907 |
| 51632 | 10: 48370815 | 51634 | 10: 48417884 | 51644 | 10: 48589378 | 51646 | 10: 48589442 | 51648 | 10: 48668493 |
| 51650 | 10: 48743669 | 51660 | 10: 49108347 | 51661 | 10: 49132750 | 51668 | 10: 49271034 | 51675 | 10: 49350595 |
| 51677 | 10: 49377221 | 51678 | 10: 49377286 | 51681 | 10: 49419982 | 51693 | 10: 49601513 | 51703 | 10: 49841256 |
| 51704 | 10: 49841837 | 51705 | 10: 49858694 | 51715 | 10: 50107685 | 51734 | 10: 50489426 | 51739 | 10: 50557454 |
| 51740 | 10: 50582984 | 51746 | 10: 50846288 | 51749 | 10: 51020894 | 51752 | 10: 51062674 | 51757 | 10: 51161988 |
| 51758 | 10: 51163453 | 51761 | 10: 51186753 | 51771 | 10: 51429834 | 51772 | 10: 51436172 | 51776 | 10: 51594174 |
| 51786 | 10: 51771324 | 51787 | 10: 51772122 | 51798 | 10: 52037658 | 51799 | 10: 52053671 | 51807 | 10: 52239885 |
| 51808 | 10: 52247304 | 51818 | 10: 52549691 | 51820 | 10: 52577136 | 51821 | 10: 52644574 | 51826 | 10: 52759280 |
| 51827 | 10: 52770881 | 51841 | 10: 52889989 | 51843 | 10: 52975439 | 51852 | 10: 53392084 | 51855 | 10: 53503163 |
| 51856 | 10: 53566836 | 51857 | 10: 53573889 | 51859 | 10: 53599523 | 51870 | 10: 54084338 | 51871 | 10: 54097737 |
| 51873 | 10: 54151043 | 51874 | 10: 54166847 | 51879 | 10: 54206436 | 51880 | 10: 54223827 | 51885 | 10: 54381985 |
| 51897 | 10: 54587863 | 51900 | 10: 54728840 | 51905 | 10: 55049581 | 51908 | 10: 55137727 | 51909 | 10: 55138450 |
| 51915 | 10: 55257378 | 51922 | 10: 55519496 | 51928 | 10: 55693450 | 51929 | 10: 55701600 | 51930 | 10: 55701734 |
| 51935 | 10: 55824628 | 51937 | 10: 55884610 | 51944 | 10: 56050425 | 51950 | 10: 56235551 | 51961 | 10: 56404358 |
| 51974 | 10: 56691062 | 52008 | 10: 57738832 | 52009 | 10: 57749028 | 52012 | 10: 57825646 | 52021 | 10: 58035128 |
| 52030 | 10: 58215720 | 52031 | 10: 58215742 | 52032 | 10: 58223900 | 52033 | 10: 58223933 | 52034 | 10: 58223992 |
| 52038 | 10: 58382992 | 52043 | 10: 58575796 | 52048 | 10: 58651910 | 52055 | 10: 58892988 | 52058 | 10: 58957454 |
| 52059 | 10: 58957455 | 52060 | 10: 58957462 | 52061 | 10: 58963341 | 52068 | 10: 59169515 | 52069 | 10: 59257696 |
| 52070 | 10: 59257779 | 52074 | 10: 59431442 | 52084 | 10: 59743414 | 52096 | 10: 60229145 | 52099 | 10: 60376158 |
| 52104 | 10: 60467179 | 52132 | 10: 61502794 | 52137 | 10: 61691415 | 52142 | 10: 61759883 | 52143 | 10: 61761276 |
| 52144 | 10: 61770176 | 52147 | 10: 61801949 | 52152 | 10: 61854226 | 52156 | 10: 61927985 | 52161 | 10: 62065140 |
| 52162 | 10: 62070975 | 52167 | 10: 62152633 | 52188 | 10: 62478919 | 52189 | 10: 62483660 | 52196 | 10: 62841361 |
| 52197 | 10: 62843919 | 52206 | 10: 63003189 | 52207 | 10: 63005018 | 52215 | 10: 63176255 | 52217 | 10: 63234235 |
| 52218 | 10: 63236141 | 52230 | 10: 63545031 | 52233 | 10: 63676002 | 52235 | 10: 63714642 | 52246 | 10: 64098165 |
| 52254 | 10: 64366724 | 52255 | 10: 64366769 | 52256 | 10: 64366784 | 52257 | 10: 64366788 | 52258 | 10: 64366808 |
| 52259 | 10: 64366813 | 52260 | 10: 64366843 | 52273 | 10: 64623835 | 52274 | 10: 64624712 | 52297 | 10: 65311772 |
| 52298 | 10: 65333523 | 52300 | 10: 65360982 | 52301 | 10: 65363558 | 52302 | 10: 65366448 | 52303 | 10: 65370682 |
| 52304 | 10: 65374137 | 52305 | 10: 65381281 | 52306 | 10: 65388071 | 52307 | 10: 65427744 | 52308 | 10: 65428255 |
| 52309 | 10: 65448723 | 52310 | 10: 65460822 | 52311 | 10: 65464926 | 52315 | 10: 65667285 | 52317 | 10: 65689392 |
| 52319 | 10: 65693892 | 52320 | 10: 65705467 | 52328 | 10: 65975774 | 52333 | 10: 66037974 | 52374 | 10: 66920995 |
| 52379 | 10: 67082677 | 52383 | 10: 67193245 | 52384 | 10: 67193602 | 52385 | 10: 67207585 | 52388 | 10: 67271539 |
| 52389 | 10: 67275755 | 52400 | 10: 67706373 | 52401 | 10: 67706382 | 52403 | 10: 67837002 | 52412 | 10: 67919519 |
| 52415 | 10: 68009703 | 52419 | 10: 68069027 | 52426 | 10: 68164248 | 52430 | 10: 68213913 | 52434 | 10: 68307915 |
| 52435 | 10: 68336932 | 52445 | 10: 68532314 | 52446 | 10: 68532338 | 52449 | 10: 68589455 | 52453 | 10: 68616316 |
| 52454 | 10: 68648835 | 52460 | 10: 68845801 | 52479 | 10: 69314335 | 52483 | 10: 69414869 | 52489 | 10: 69550619 |
| 52497 | 10: 69813170 | 52506 | 10: 69985799 | 52518 | 10: 70311136 | 52530 | 10: 70914151 | 52532 | 10: 70933695 |
| 52543 | 10: 71144557 | 52545 | 10: 71190058 | 52547 | 10: 71282860 | 52549 | 10: 71395114 | 52550 | 10: 71401946 |
| 52553 | 10: 71492246 | 52560 | 10: 71739567 | 52561 | 10: 71739616 | 52562 | 10: 71741589 | 52568 | 10: 71990029 |
| 52594 | 10: 72844951 | 52601 | 10: 73180235 | 52603 | 10: 73207644 | 52608 | 10: 73381610 | 52619 | 10: 73593430 |
| 52621 | 10: 73621273 | 52625 | 10: 73665667 | 52628 | 10: 73702186 | 52631 | 10: 73800772 | 52634 | 10: 73889706 |
| 52639 | 10: 74014694 | 52640 | 10: 74014740 | 52647 | 10: 74278788 | 52669 | 10: 75144155 | 52684 | 10: 75472993 |
| 52690 | 10: 75648302 | 52702 | 10: 76063641 | 52711 | 10: 76208279 | 52712 | 10: 76213571 | 52713 | 10: 76216092 |
| 52714 | 10: 76217503 | 52716 | 10: 76219150 | 52717 | 10: 76231514 | 52727 | 10: 76445843 | 52739 | 10: 76742309 |
| 52740 | 10: 76780386 | 52750 | 10: 76927979 | 52753 | 10: 77027256 | 52754 | 10: 77038645 | 52755 | 10: 77043267 |
| 52756 | 10: 77050549 | 52757 | 10: 77063990 | 52758 | 10: 77079521 | 52760 | 10: 77086192 | 52761 | 10: 77087944 |
| 52764 | 10: 77165808 | 52765 | 10: 77182957 | 52769 | 10: 77284463 | 52770 | 10: 77295759 | 52772 | 10: 77310958 |
| 52783 | 10: 77734390 | 52785 | 10: 77815166 | 52787 | 10: 77843400 | 52790 | 10: 77908553 | 52794 | 10: 77934474 |
| 52800 | 10: 78024784 | 52804 | 10: 78090692 | 52805 | 10: 78091383 | 52806 | 10: 78170657 | 52818 | 10: 78466873 |
| 52819 | 10: 78489026 | 52820 | 10: 78524325 | 52833 | 10: 78874750 | 52834 | 10: 78890533 | 52843 | 10: 79142128 |
| 52844 | 10: 79142222 | 52850 | 10: 79364696 | 52858 | 10: 79552918 | 52864 | 10: 79765450 | 52866 | 10: 79778751 |
| 52876 | 10: 80036481 | 52878 | 10: 80058483 | 52887 | 10: 80321841 | 52888 | 10: 80354807 | 52890 | 10: 80376119 |
| 52897 | 10: 80516438 | 52898 | 10: 80584353 | 52899 | 10: 80612799 | 52908 | 10: 80833008 | 52917 | 10: 81113106 |
| 52918 | 10: 81113113 | 52919 | 10: 81113154 | 52920 | 10: 81119799 | 52921 | 10: 81122203 | 52922 | 10: 81122225 |
| 52923 | 10: 81133087 | 52924 | 10: 81134221 | 52925 | 10: 81141810 | 52926 | 10: 81147906 | 52927 | 10: 81159861 |
| 52928 | 10: 81163746 | 52929 | 10: 81169707 | 52930 | 10: 81171404 | 52931 | 10: 81174589 | 52932 | 10: 81177743 |
| 52933 | 10: 81179444 | 52934 | 10: 81180179 | 52935 | 10: 81182426 | 52936 | 10: 81188153 | 52937 | 10: 81191624 |
| 52938 | 10: 81192985 | 52939 | 10: 81201260 | 52942 | 10: 81232104 | 52946 | 10: 81275274 | 52948 | 10: 81293569 |
| 52949 | 10: 81313921 | 52954 | 10: 81424405 | 52955 | 10: 81429276 | 52956 | 10: 81433859 | 52957 | 10: 81434033 |
| 52960 | 10: 81522739 | 52961 | 10: 81563853 | 52971 | 10: 81760421 | 52983 | 10: 81884426 | 52985 | 10: 81907059 |
| 52989 | 10: 82020430 | 52991 | 10: 82046798 | 52993 | 10: 82113076 | 52994 | 10: 82117432 | 52996 | 10: 82155665 |
| 53001 | 10: 82226992 | 53003 | 10: 82262769 | 53010 | 10: 82436819 | 53020 | 10: 82813082 | 53031 | 10: 83035771 |
| 53036 | 10: 83168495 | 53037 | 10: 83180678 | 53043 | 10: 83268162 | 53064 | 10: 83783323 | 53074 | 10: 83967539 |
| 53078 | 10: 84046423 | 53080 | 10: 84074336 | 53081 | 10: 84113189 | 53087 | 10: 84235120 | 53088 | 10: 84249302 |
| 53091 | 10: 84274673 | 53093 | 10: 84321770 | 53095 | 10: 84358684 | 53098 | 10: 84410074 | 53101 | 10: 84432430 |
| 53113 | 10: 84804439 | 53114 | 10: 84809599 | 53117 | 10: 84921371 | 53120 | 10: 84937878 | 53122 | 10: 84972916 |
| 53124 | 10: 85090540 | 53125 | 10: 85090775 | 53126 | 10: 85093417 | 53129 | 10: 85140109 | 53135 | 10: 85210872 |
| 53139 | 10: 85277645 | 53144 | 10: 85348496 | 53148 | 10: 85452360 | 53159 | 10: 85646771 | 53161 | 10: 85685359 |
| 53162 | 10: 85714779 | 53163 | 10: 85731082 | 53175 | 10: 86198306 | 53179 | 10: 86295239 | 53180 | 10: 86322591 |
| 53182 | 10: 86350641 | 53183 | 10: 86373524 | 53186 | 10: 86404676 | 53188 | 10: 86427269 | 53191 | 10: 86481134 |
| 53193 | 10: 86492591 | 53194 | 10: 86510757 | 53196 | 10: 86545729 | 53197 | 10: 86553401 | 53207 | 10: 86743362 |
| 53209 | 10: 86835796 | 53211 | 10: 86863386 | 53214 | 10: 86939644 | 53223 | 10: 87113458 | 53244 | 10: 87621927 |
| 53246 | 10: 87627438 | 53250 | 10: 87721184 | 53252 | 10: 87755864 | 53256 | 10: 87849396 | 53257 | 10: 87849463 |
| 53258 | 10: 87850909 | 53268 | 10: 88187681 | 53270 | 10: 88260182 | 53271 | 10: 88298292 | 53274 | 10: 88347395 |
| 53276 | 10: 88380317 | 53280 | 10: 88522805 | 53291 | 10: 88839037 | 53292 | 10: 88845164 | 53293 | 10: 88846191 |
| 53297 | 10: 88888537 | 53299 | 10: 88925587 | 53313 | 10: 89160492 | 53314 | 10: 89160521 | 53316 | 10: 89233565 |
| 53331 | 10: 89626724 | 53332 | 10: 89626819 | 53343 | 10: 89798617 | 53348 | 10: 89872058 | 53349 | 10: 89872136 |
| 53350 | 10: 89933077 | 53364 | 10: 90186331 | 53378 | 10: 90418700 | 53379 | 10: 90418707 | 53415 | 10: 90987301 |
| 53419 | 10: 91011354 | 53420 | 10: 91019729 | 53431 | 10: 91348121 | 53444 | 10: 91737619 | 53445 | 10: 91746178 |
| 53449 | 10: 91823694 | 53456 | 10: 92080780 | 53458 | 10: 92100804 | 53464 | 10: 92232897 | 53465 | 10: 92281587 |
| 53473 | 10: 92466754 | 53476 | 10: 92499113 | 53480 | 10: 92594602 | 53482 | 10: 92604984 | 53488 | 10: 92710477 |
| 53489 | 10: 92717162 | 53492 | 10: 92799252 | 53507 | 10: 93053048 | 53508 | 10: 93053064 | 53512 | 10: 93140659 |
| 53528 | 10: 93626548 | 53537 | 10: 93975527 | 53538 | 10: 93977121 | 53549 | 10: 94274383 | 53557 | 10: 94377341 |
| 53558 | 10: 94377386 | 53572 | 10: 94695544 | 53576 | 10: 94786454 | 53584 | 10: 95064914 | 53595 | 10: 95385183 |
| 53597 | 10: 95412469 | 53602 | 10: 95561991 | 53603 | 10: 95575347 | 53608 | 10: 95643374 | 53609 | 10: 95672545 |
| 53611 | 10: 95688501 | 53613 | 10: 95728606 | 53618 | 10: 95843138 | 53620 | 10: 95843176 | 53628 | 10: 96029745 |
| 53635 | 10: 96240911 | 53638 | 10: 96288699 | 53641 | 10: 96380513 | 53642 | 10: 96380599 | 53647 | 10: 96569247 |
| 53650 | 10: 96598589 | 53657 | 10: 96839484 | 53659 | 10: 96875821 | 53662 | 10: 96976508 | 53673 | 10: 97153819 |
| 53678 | 10: 97206516 | 53683 | 10: 97309948 | 53684 | 10: 97309954 | 53692 | 10: 97454671 | 53699 | 10: 97550303 |
| 53700 | 10: 97582963 | 53749 | 10: 98562531 | 53777 | 10: 99221264 | 53788 | 10: 99540676 | 53789 | 10: 99540722 |
| 53790 | 10: 99540732 | 53791 | 10: 99540816 | 53792 | 10: 99540849 | 53805 | 10: 99922832 | 53806 | 10: 99926816 |
| 53808 | 10: 99941485 | 53812 | 10: 10001067 | 53815 | 10: 10008239 | 53819 | 10: 10018045 | 53820 | 10: 10018777 |
| 53822 | 10: 10024360 | 53825 | 10: 10029136 | 53826 | 10: 10030147 | 53827 | 10: 10030338 | 53828 | 10: 10030391 |
| 53829 | 10: 10030447 | 53831 | 10: 10033080 | 53852 | 10: 10082168 | 53854 | 10: 10087653 | 53858 | 10: 10096073 |
| 53860 | 10: 10096399 | 53861 | 10: 10097431 | 53871 | 10: 10113021 | 53872 | 10: 10113562 | 53888 | 10: 10143538 |
| 53889 | 10: 10147495 | 53890 | 10: 10148861 | 53891 | 10: 10150021 | 53892 | 10: 10150656 | 53903 | 10: 10170837 |
| 53912 | 10: 10180575 | 53918 | 10: 10186280 | 53919 | 10: 10187041 | 53924 | 10: 10200983 | 53934 | 10: 10213583 |
| 53937 | 10: 10221227 | 53938 | 10: 10222090 | 53940 | 10: 10225283 | 53947 | 10: 10236108 | 53950 | 10: 10248171 |
| 53959 | 10: 10271339 | 53962 | 10: 10281920 | 54004 | 11: 441458 | 54008 | 11: 588104 | 54009 | 11: 612918 |
| 54010 | 11: 620469 | 54013 | 11: 675675 | 54015 | 11: 721258 | 54018 | 11: 743177 | 54023 | 11: 818693 |
| 54024 | 11: 818745 | 54025 | 11: 839682 | 54028 | 11: 884789 | 54029 | 11: 916613 | 54042 | 11: 1140277 |
| 54051 | 11: 1252661 | 54072 | 11: 1599964 | 54073 | 11: 1603522 | 54074 | 11: 1604088 | 54076 | 11: 1610503 |
| 54077 | 11: 1613029 | 54078 | 11: 1613069 | 54079 | 11: 1615691 | 54080 | 11: 1615825 | 54081 | 11: 1615860 |
| 54082 | 11: 1616045 | 54083 | 11: 1617470 | 54084 | 11: 1617491 | 54085 | 11: 1618013 | 54088 | 11: 1661775 |
| 54090 | 11: 1692551 | 54091 | 11: 1692581 | 54092 | 11: 1692586 | 54094 | 11: 1715577 | 54105 | 11: 1929102 |
| 54106 | 11: 1939516 | 54109 | 11: 1975548 | 54112 | 11: 2019047 | 54113 | 11: 2019051 | 54116 | 11: 2054152 |
| 54117 | 11: 2058128 | 54122 | 11: 2135119 | 54123 | 11: 2165271 | 54124 | 11: 2165552 | 54125 | 11: 2165653 |
| 54135 | 11: 2371904 | 54136 | 11: 2375767 | 54137 | 11: 2379791 | 54138 | 11: 2380701 | 54139 | 11: 2381359 |
| 54141 | 11: 2381820 | 54142 | 11: 2381938 | 54143 | 11: 2382261 | 54144 | 11: 2383322 | 54145 | 11: 2383432 |
| 54147 | 11: 2388794 | 54148 | 11: 2391923 | 54149 | 11: 2392633 | 54150 | 11: 2392687 | 54151 | 11: 2392744 |
| 54152 | 11: 2393551 | 54153 | 11: 2393601 | 54154 | 11: 2393614 | 54155 | 11: 2394563 | 54156 | 11: 2394565 |
| 54157 | 11: 2394754 | 54158 | 11: 2429820 | 54159 | 11: 2433742 | 54162 | 11: 2466683 | 54163 | 11: 2466765 |
| 54164 | 11: 2466767 | 54176 | 11: 2690324 | 54177 | 11: 2691733 | 54179 | 11: 2704089 | 54185 | 11: 2887185 |
| 54186 | 11: 2909443 | 54190 | 11: 2965363 | 54191 | 11: 2970198 | 54192 | 11: 2971406 | 54193 | 11: 2974009 |
| 54194 | 11: 3048024 | 54195 | 11: 3048066 | 54197 | 11: 3068135 | 54198 | 11: 3073771 | 54201 | 11: 3080087 |
| 54202 | 11: 3080140 | 54203 | 11: 3080321 | 54204 | 11: 3080329 | 54212 | 11: 3324474 | 54214 | 11: 3339436 |
| 54222 | 11: 3496384 | 54223 | 11: 3498026 | 54229 | 11: 3630171 | 54232 | 11: 3683284 | 54234 | 11: 3687500 |
| 54235 | 11: 3697811 | 54236 | 11: 3703164 | 54241 | 11: 3827658 | 54242 | 11: 3831757 | 54243 | 11: 3831764 |
| 54244 | 11: 3833403 | 54245 | 11: 3834470 | 54246 | 11: 3834544 | 54249 | 11: 3860402 | 54250 | 11: 3861478 |
| 54251 | 11: 3861495 | 54252 | 11: 3863319 | 54258 | 11: 4044668 | 54261 | 11: 4144540 | 54274 | 11: 4626193 |
| 54275 | 11: 4643628 | 54277 | 11: 4692004 | 54278 | 11: 4715218 | 54280 | 11: 4754025 | 54285 | 11: 4846290 |
| 54288 | 11: 4908167 | 54301 | 11: 5122618 | 54306 | 11: 5207458 | 54307 | 11: 5208484 | 54308 | 11: 5216864 |
| 54317 | 11: 5475369 | 54320 | 11: 5522837 | 54321 | 11: 5546069 | 54322 | 11: 5547645 | 54336 | 11: 5892298 |
| 54338 | 11: 5921461 | 54342 | 11: 6014407 | 54344 | 11: 6057385 | 54346 | 11: 6090351 | 54353 | 11: 6281176 |
| 54367 | 11: 6672389 | 54371 | 11: 6825527 | 54378 | 11: 7079505 | 54379 | 11: 7079550 | 54383 | 11: 7226247 |
| 54385 | 11: 7290568 | 54387 | 11: 7312202 | 54390 | 11: 7398056 | 54392 | 11: 7442481 | 54395 | 11: 7492967 |
| 54397 | 11: 7506651 | 54406 | 11: 7612707 | 54424 | 11: 8016040 | 54441 | 11: 8559109 | 54456 | 11: 9088843 |
| 54457 | 11: 9094665 | 54458 | 11: 9108482 | 54459 | 11: 9116906 | 54460 | 11: 9122303 | 54461 | 11: 9124299 |
| 54462 | 11: 9125436 | 54463 | 11: 9129918 | 54464 | 11: 9140181 | 54465 | 11: 9142056 | 54467 | 11: 9146703 |
| 54468 | 11: 9147988 | 54469 | 11: 9149728 | 54470 | 11: 9150188 | 54471 | 11: 9150797 | 54472 | 11: 9152234 |
| 54473 | 11: 9153632 | 54477 | 11: 9263561 | 54485 | 11: 9432577 | 54493 | 11: 9646962 | 54497 | 11: 9717817 |
| 54499 | 11: 9759593 | 54502 | 11: 9782467 | 54509 | 11: 9926241 | 54516 | 11: 10139900 | 54517 | 11: 10192082 |
| 54518 | 11: 10205888 | 54519 | 11: 10207241 | 54521 | 11: 10218640 | 54522 | 11: 10223170 | 54528 | 11: 10378672 |
| 54534 | 11: 10498811 | 54535 | 11: 10506565 | 54539 | 11: 10583951 | 54542 | 11: 10706952 | 54543 | 11: 10730447 |
| 54545 | 11: 10770592 | 54547 | 11: 10787415 | 54548 | 11: 10804935 | 54549 | 11: 10814646 | 54551 | 11: 10817795 |
| 54559 | 11: 11049635 | 54562 | 11: 11215341 | 54564 | 11: 11231984 | 54565 | 11: 11242289 | 54573 | 11: 11352441 |
| 54574 | 11: 11362173 | 54576 | 11: 11392077 | 54578 | 11: 11435064 | 54584 | 11: 11556298 | 54586 | 11: 11616272 |
| 54601 | 11: 12092660 | 54602 | 11: 12096347 | 54608 | 11: 12209379 | 54623 | 11: 12569024 | 54625 | 11: 12600200 |
| 54634 | 11: 12790767 | 54643 | 11: 13030163 | 54645 | 11: 13087614 | 54648 | 11: 13127507 | 54662 | 11: 13410435 |
| 54667 | 11: 13471629 | 54693 | 11: 14069498 | 54697 | 11: 14240512 | 54700 | 11: 14312963 | 54707 | 11: 14569196 |
| 54708 | 11: 14601805 | 54717 | 11: 14951793 | 54720 | 11: 15049901 | 54729 | 11: 15188618 | 54737 | 11: 15351949 |
| 54800 | 11: 16839124 | 54806 | 11: 17005906 | 54808 | 11: 17038164 | 54809 | 11: 17038230 | 54811 | 11: 17062733 |
| 54819 | 11: 17340238 | 54820 | 11: 17340240 | 54821 | 11: 17361542 | 54823 | 11: 17465587 | 54829 | 11: 17736027 |
| 54830 | 11: 17736142 | 54870 | 11: 18811490 | 54871 | 11: 18834617 | 54879 | 11: 19016954 | 54885 | 11: 19227167 |
| 54891 | 11: 19479022 | 54893 | 11: 19516116 | 54894 | 11: 19516281 | 54897 | 11: 19574521 | 54909 | 11: 20009995 |
| 54914 | 11: 20249271 | 54916 | 11: 20268750 | 54929 | 11: 20671302 | 54933 | 11: 20761364 | 54935 | 11: 20839310 |
| 54946 | 11: 21100669 | 54948 | 11: 21153847 | 54952 | 11: 21298331 | 54953 | 11: 21386222 | 54965 | 11: 21549331 |
| 54968 | 11: 21639708 | 54971 | 11: 21679170 | 54979 | 11: 21792131 | 54985 | 11: 21911853 | 54998 | 11: 22232061 |
| 54999 | 11: 22277590 | 55009 | 11: 22536318 | 55016 | 11: 22632697 | 55031 | 11: 23047670 | 55035 | 11: 23109116 |
| 55043 | 11: 23406318 | 55045 | 11: 23420733 | 55057 | 11: 23678147 | 55059 | 11: 23713701 | 55060 | 11: 23740579 |
| 55061 | 11: 23792727 | 55062 | 11: 23799914 | 55072 | 11: 23927783 | 55073 | 11: 23927808 | 55082 | 11: 24309077 |
| 55114 | 11: 24847692 | 55115 | 11: 24847723 | 55120 | 11: 25005279 | 55143 | 11: 25650622 | 55144 | 11: 25668988 |
| 55155 | 11: 26000399 | 55158 | 11: 26101828 | 55163 | 11: 26155816 | 55171 | 11: 26293504 | 55172 | 11: 26294033 |
| 55175 | 11: 26405127 | 55176 | 11: 26406745 | 55179 | 11: 26441367 | 55184 | 11: 26597807 | 55202 | 11: 27150645 |
| 55210 | 11: 27309958 | 55223 | 11: 27583040 | 55224 | 11: 27612153 | 55225 | 11: 27648326 | 55226 | 11: 27664648 |
| 55227 | 11: 27673338 | 55235 | 11: 27863723 | 55243 | 11: 28050164 | 55244 | 11: 28054041 | 55254 | 11: 28269292 |
| 55255 | 11: 28312159 | 55256 | 11: 28314792 | 55259 | 11: 28347672 | 55261 | 11: 28406003 | 55270 | 11: 28648699 |
| 55271 | 11: 28677976 | 55272 | 11: 28697612 | 55275 | 11: 28750175 | 55288 | 11: 28998567 | 55292 | 11: 29029776 |
| 55294 | 11: 29094181 | 55305 | 11: 29333853 | 55306 | 11: 29339273 | 55311 | 11: 29433875 | 55312 | 11: 29446589 |
| 55314 | 11: 29483268 | 55316 | 11: 29523821 | 55320 | 11: 29644262 | 55321 | 11: 29652000 | 55328 | 11: 29750755 |
| 55338 | 11: 29944937 | 55339 | 11: 30022269 | 55347 | 11: 30283476 | 55357 | 11: 30480608 | 55358 | 11: 30487514 |
| 55359 | 11: 30490953 | 55360 | 11: 30498423 | 55363 | 11: 30695691 | 55371 | 11: 30816430 | 55375 | 11: 30852665 |
| 55378 | 11: 30909842 | 55381 | 11: 31003265 | 55386 | 11: 31096059 | 55394 | 11: 31338792 | 55395 | 11: 31338818 |
| 55396 | 11: 31338832 | 55418 | 11: 31989218 | 55419 | 11: 32020262 | 55427 | 11: 32309370 | 55440 | 11: 32659947 |
| 55451 | 11: 33038916 | 55454 | 11: 33061320 | 55455 | 11: 33063315 | 55465 | 11: 33230608 | 55473 | 11: 33483917 |
| 55497 | 11: 34161881 | 55512 | 11: 34992407 | 55515 | 11: 35028247 | 55518 | 11: 35081009 | 55523 | 11: 35271551 |
| 55550 | 11: 35973444 | 55555 | 11: 36103640 | 55567 | 11: 36415088 | 55575 | 11: 36677962 | 55578 | 11: 36729555 |
| 55579 | 11: 36730226 | 55584 | 11: 36935209 | 55590 | 11: 37042393 | 55592 | 11: 37065356 | 55593 | 11: 37084548 |
| 55597 | 11: 37151685 | 55602 | 11: 37273838 | 55607 | 11: 37321094 | 55610 | 11: 37357247 | 55617 | 11: 37555217 |
| 55622 | 11: 37622807 | 55623 | 11: 37622964 | 55629 | 11: 37686699 | 55631 | 11: 37699061 | 55632 | 11: 37708915 |
| 55639 | 11: 37993013 | 55640 | 11: 38023147 | 55644 | 11: 38167213 | 55652 | 11: 38524637 | 55676 | 11: 39146746 |
| 55698 | 11: 39953399 | 55719 | 11: 40780484 | 55720 | 11: 40781022 | 55725 | 11: 40977806 | 55729 | 11: 41081305 |
| 55731 | 11: 41095065 | 55732 | 11: 41108333 | 55733 | 11: 41112618 | 55734 | 11: 41119005 | 55735 | 11: 41119970 |
| 55737 | 11: 41122253 | 55738 | 11: 41130588 | 55739 | 11: 41131422 | 55740 | 11: 41138457 | 55742 | 11: 41163401 |
| 55743 | 11: 41165333 | 55744 | 11: 41172319 | 55745 | 11: 41190502 | 55746 | 11: 41211212 | 55747 | 11: 41236765 |
| 55757 | 11: 41353565 | 55785 | 11: 42015177 | 55813 | 11: 42568364 | 55819 | 11: 42700906 | 55838 | 11: 43239984 |
| 55862 | 11: 43925979 | 55864 | 11: 44029565 | 55869 | 11: 44191991 | 55875 | 11: 44335233 | 55877 | 11: 44398012 |
| 55884 | 11: 44616476 | 55885 | 11: 44669598 | 55886 | 11: 44720675 | 55891 | 11: 44864177 | 55892 | 11: 44872149 |
| 55895 | 11: 44907130 | 55896 | 11: 44910831 | 55897 | 11: 44915340 | 55899 | 11: 44947747 | 55900 | 11: 44960989 |
| 55901 | 11: 44979580 | 55905 | 11: 45085243 | 55910 | 11: 45233285 | 55912 | 11: 45240869 | 55926 | 11: 45875224 |
| 55930 | 11: 45909750 | 55932 | 11: 45965043 | 55935 | 11: 46005981 | 55939 | 11: 46042064 | 55944 | 11: 46118783 |
| 55948 | 11: 46189212 | 55966 | 11: 46586822 | 55984 | 11: 46788369 | 55985 | 11: 46796356 | 55986 | 11: 46801200 |
| 55990 | 11: 46887994 | 55991 | 11: 46893010 | 55992 | 11: 46895379 | 55993 | 11: 46895384 | 55994 | 11: 46895386 |
| 55995 | 11: 46895443 | 55996 | 11: 46896988 | 55997 | 11: 46914387 | 55998 | 11: 46915926 | 56001 | 11: 46933431 |
| 56002 | 11: 46946293 | 56009 | 11: 47133868 | 56010 | 11: 47136307 | 56012 | 11: 47153161 | 56016 | 11: 47318477 |
| 56020 | 11: 47346660 | 56023 | 11: 47402450 | 56025 | 11: 47421701 | 56028 | 11: 47484468 | 56039 | 11: 47868107 |
| 56041 | 11: 47957513 | 56051 | 11: 48153310 | 56052 | 11: 48179620 | 56055 | 11: 48232533 | 56057 | 11: 48284436 |
| 56064 | 11: 48495828 | 56065 | 11: 48519034 | 56066 | 11: 48519745 | 56077 | 11: 48624594 | 56078 | 11: 48641618 |
| 56079 | 11: 48643769 | 56080 | 11: 48648683 | 56081 | 11: 48652079 | 56082 | 11: 48653016 | 56083 | 11: 48653703 |
| 56084 | 11: 48659221 | 56086 | 11: 48661468 | 56087 | 11: 48662095 | 56088 | 11: 48663888 | 56089 | 11: 48664547 |
| 56090 | 11: 48665440 | 56091 | 11: 48666103 | 56092 | 11: 48666658 | 56093 | 11: 48673397 | 56094 | 11: 48673919 |
| 56095 | 11: 48674533 | 56096 | 11: 48675652 | 56097 | 11: 48676148 | 56098 | 11: 48677162 | 56099 | 11: 48679806 |
| 56100 | 11: 48680593 | 56101 | 11: 48682804 | 56102 | 11: 48685811 | 56103 | 11: 48690618 | 56104 | 11: 48695696 |
| 56105 | 11: 48703362 | 56107 | 11: 48712410 | 56108 | 11: 48715777 | 56109 | 11: 48729623 | 56110 | 11: 48732364 |
| 56111 | 11: 48740101 | 56112 | 11: 48740643 | 56113 | 11: 48742231 | 56114 | 11: 48745355 | 56115 | 11: 48748086 |
| 56116 | 11: 48757634 | 56117 | 11: 48788737 | 56118 | 11: 48792299 | 56120 | 11: 48910719 | 56134 | 11: 49167307 |
| 56142 | 11: 49394511 | 56144 | 11: 49406127 | 56146 | 11: 49419581 | 56151 | 11: 49574160 | 56163 | 11: 49832758 |
| 56164 | 11: 49852553 | 56165 | 11: 49852648 | 56200 | 11: 51131191 | 56203 | 11: 51184125 | 56220 | 11: 51745809 |
| 56243 | 11: 52697360 | 56265 | 11: 53468115 | 56274 | 11: 53833063 | 56290 | 11: 54327571 | 56298 | 11: 54566473 |
| 56300 | 11: 54590112 | 56320 | 11: 54776336 | 56323 | 11: 54807232 | 56328 | 11: 54930465 | 56329 | 11: 54961590 |
| 56340 | 11: 55186748 | 56347 | 11: 55369267 | 56355 | 11: 55552815 | 56356 | 11: 55571320 | 56363 | 11: 55683959 |
| 56364 | 11: 55696252 | 56378 | 11: 56190217 | 56402 | 11: 56970533 | 56428 | 11: 57877493 | 56439 | 11: 58140156 |
| 56493 | 11: 59622606 | 56497 | 11: 59747111 | 56500 | 11: 59861239 | 56510 | 11: 60271069 | 56511 | 11: 60271074 |
| 56512 | 11: 60271117 | 56513 | 11: 60271193 | 56520 | 11: 60465745 | 56521 | 11: 60465915 | 56522 | 11: 60465920 |
| 56523 | 11: 60465923 | 56524 | 11: 60468952 | 56525 | 11: 60470373 | 56526 | 11: 60470388 | 56527 | 11: 60471668 |
| 56529 | 11: 60472081 | 56530 | 11: 60474103 | 56531 | 11: 60474134 | 56532 | 11: 60474983 | 56533 | 11: 60475508 |
| 56534 | 11: 60475664 | 56535 | 11: 60478021 | 56536 | 11: 60478028 | 56537 | 11: 60478049 | 56538 | 11: 60478204 |
| 56540 | 11: 60494769 | 56541 | 11: 60494791 | 56542 | 11: 60517676 | 56543 | 11: 60566671 | 56544 | 11: 60567347 |
| 56545 | 11: 60567387 | 56546 | 11: 60616886 | 56549 | 11: 60641170 | 56550 | 11: 60641182 | 56553 | 11: 60671076 |
| 56557 | 11: 60758092 | 56558 | 11: 60775322 | 56560 | 11: 60906972 | 56562 | 11: 60949119 | 56564 | 11: 60961745 |
| 56566 | 11: 61011277 | 56567 | 11: 61011512 | 56570 | 11: 61079196 | 56573 | 11: 61139067 | 56574 | 11: 61149300 |
| 56575 | 11: 61149340 | 56576 | 11: 61149341 | 56578 | 11: 61149398 | 56579 | 11: 61149409 | 56581 | 11: 61172809 |
| 56585 | 11: 61361997 | 56588 | 11: 61539166 | 56590 | 11: 61548155 | 56606 | 11: 62153933 | 56616 | 11: 62410952 |
| 56621 | 11: 62549111 | 56627 | 11: 62639185 | 56629 | 11: 62703391 | 56637 | 11: 62848946 | 56645 | 11: 63079035 |
| 56647 | 11: 63132879 | 56657 | 11: 63363010 | 56658 | 11: 63376645 | 56661 | 11: 63394494 | 56665 | 11: 63456586 |
| 56671 | 11: 63645957 | 56672 | 11: 63664952 | 56676 | 11: 63733057 | 56679 | 11: 63799850 | 56687 | 11: 63862724 |
| 56691 | 11: 63954830 | 56693 | 11: 63980633 | 56694 | 11: 63980697 | 56695 | 11: 64007710 | 56708 | 11: 64345353 |
| 56712 | 11: 64472503 | 56716 | 11: 64576376 | 56718 | 11: 64684536 | 56721 | 11: 64807816 | 56726 | 11: 64972478 |
| 56735 | 11: 65170349 | 56736 | 11: 65170393 | 56741 | 11: 65341010 | 56743 | 11: 65372142 | 56746 | 11: 65438305 |
| 56766 | 11: 65998851 | 56769 | 11: 66070592 | 56775 | 11: 66159407 | 56782 | 11: 66307327 | 56783 | 11: 66315381 |
| 56790 | 11: 66473480 | 56795 | 11: 66556801 | 56796 | 11: 66556834 | 56799 | 11: 66629962 | 56813 | 11: 67084838 |
| 56814 | 11: 67108786 | 56815 | 11: 67114080 | 56821 | 11: 67213238 | 56829 | 11: 67439320 | 56832 | 11: 67453087 |
| 56834 | 11: 67479748 | 56835 | 11: 67481439 | 56836 | 11: 67483125 | 56838 | 11: 67512057 | 56842 | 11: 67544307 |
| 56843 | 11: 67549232 | 56844 | 11: 67549363 | 56846 | 11: 67556514 | 56847 | 11: 67563313 | 56848 | 11: 67582936 |
| 56849 | 11: 67582999 | 56850 | 11: 67583014 | 56852 | 11: 67588734 | 56855 | 11: 67702445 | 56856 | 11: 67709590 |
| 56857 | 11: 67727467 | 56858 | 11: 67728102 | 56859 | 11: 67730113 | 56860 | 11: 67736102 | 56861 | 11: 67737073 |
| 56862 | 11: 67747241 | 56863 | 11: 67749685 | 56864 | 11: 67752237 | 56865 | 11: 67759745 | 56867 | 11: 67762854 |
| 56868 | 11: 67767430 | 56869 | 11: 67772466 | 56870 | 11: 67776969 | 56871 | 11: 67787768 | 56872 | 11: 67799383 |
| 56873 | 11: 67805662 | 56874 | 11: 67810598 | 56879 | 11: 67896985 | 56882 | 11: 67970890 | 56885 | 11: 68111905 |
| 56893 | 11: 68411391 | 56897 | 11: 68467750 | 56909 | 11: 68787390 | 56921 | 11: 69071509 | 56932 | 11: 69177619 |
| 56939 | 11: 69288577 | 56941 | 11: 69302814 | 56947 | 11: 69479974 | 56950 | 11: 69514045 | 56953 | 11: 69570750 |
| 56954 | 11: 69572243 | 56956 | 11: 69619243 | 56957 | 11: 69620020 | 56958 | 11: 69620110 | 56960 | 11: 69662827 |
| 56967 | 11: 69745012 | 56992 | 11: 70292588 | 56994 | 11: 70341851 | 56995 | 11: 70350355 | 57003 | 11: 70595154 |
| 57010 | 11: 70714155 | 57011 | 11: 70718274 | 57013 | 11: 70755284 | 57014 | 11: 70761156 | 57015 | 11: 70761179 |
| 57016 | 11: 70761872 | 57017 | 11: 70762170 | 57018 | 11: 70762689 | 57019 | 11: 70762700 | 57020 | 11: 70762702 |
| 57021 | 11: 70762781 | 57022 | 11: 70762862 | 57023 | 11: 70762868 | 57024 | 11: 70763022 | 57025 | 11: 70763076 |
| 57026 | 11: 70763145 | 57027 | 11: 70763553 | 57028 | 11: 70763715 | 57029 | 11: 70763906 | 57030 | 11: 70764185 |
| 57031 | 11: 70764539 | 57032 | 11: 70768163 | 57033 | 11: 70768281 | 57034 | 11: 70769360 | 57035 | 11: 70769405 |
| 57036 | 11: 70775819 | 57037 | 11: 70782767 | 57038 | 11: 70782825 | 57039 | 11: 70806849 | 57040 | 11: 70807701 |
| 57041 | 11: 70810781 | 57043 | 11: 70862638 | 57048 | 11: 71062901 | 57049 | 11: 71062934 | 57053 | 11: 71130818 |
| 57054 | 11: 71131623 | 57057 | 11: 71142342 | 57059 | 11: 71149248 | 57066 | 11: 71175312 | 57067 | 11: 71177865 |
| 57068 | 11: 71178203 | 57070 | 11: 71182683 | 57072 | 11: 71198214 | 57073 | 11: 71199013 | 57074 | 11: 71208751 |
| 57081 | 11: 71398446 | 57094 | 11: 71666080 | 57095 | 11: 71713947 | 57099 | 11: 71827640 | 57102 | 11: 71961107 |
| 57103 | 11: 71974375 | 57108 | 11: 72101578 | 57112 | 11: 72181248 | 57128 | 11: 72810370 | 57133 | 11: 72851670 |
| 57144 | 11: 72988812 | 57146 | 11: 73025557 | 57148 | 11: 73068186 | 57150 | 11: 73079672 | 57151 | 11: 73079767 |
| 57152 | 11: 73084466 | 57153 | 11: 73087461 | 57154 | 11: 73089558 | 57155 | 11: 73090095 | 57158 | 11: 73133312 |
| 57159 | 11: 73148896 | 57161 | 11: 73219332 | 57163 | 11: 73277111 | 57166 | 11: 73438358 | 57167 | 11: 73480475 |
| 57177 | 11: 73781192 | 57181 | 11: 73853326 | 57185 | 11: 73967619 | 57188 | 11: 74009513 | 57190 | 11: 74018063 |
| 57194 | 11: 74054528 | 57205 | 11: 74195149 | 57206 | 11: 74204674 | 57207 | 11: 74205573 | 57209 | 11: 74245610 |
| 57213 | 11: 74399369 | 57218 | 11: 74544795 | 57227 | 11: 74974483 | 57230 | 11: 75017890 | 57232 | 11: 75044406 |
| 57235 | 11: 75106218 | 57236 | 11: 75198521 | 57242 | 11: 75328149 | 57243 | 11: 75330425 | 57245 | 11: 75334844 |
| 57246 | 11: 75384323 | 57249 | 11: 75420509 | 57250 | 11: 75420581 | 57251 | 11: 75477129 | 57252 | 11: 75482256 |
| 57254 | 11: 75493333 | 57256 | 11: 75509869 | 57257 | 11: 75513821 | 57266 | 11: 75686867 | 57273 | 11: 75929380 |
| 57280 | 11: 76017351 | 57309 | 11: 76876371 | 57310 | 11: 76952531 | 57321 | 11: 77091873 | 57322 | 11: 77092007 |
| 57323 | 11: 77121176 | 57324 | 11: 77123614 | 57325 | 11: 77124254 | 57327 | 11: 77132776 | 57332 | 11: 77262392 |
| 57355 | 11: 77817486 | 57362 | 11: 77896861 | 57363 | 11: 77897300 | 57364 | 11: 77897480 | 57365 | 11: 77897481 |
| 57366 | 11: 77897524 | 57367 | 11: 77899208 | 57368 | 11: 77899796 | 57369 | 11: 77900345 | 57370 | 11: 77900437 |
| 57371 | 11: 77900488 | 57372 | 11: 77900522 | 57373 | 11: 77901876 | 57374 | 11: 77901997 | 57375 | 11: 77916472 |
| 57376 | 11: 77916653 | 57377 | 11: 77917793 | 57378 | 11: 77918799 | 57379 | 11: 77920101 | 57380 | 11: 77920618 |
| 57381 | 11: 77920740 | 57382 | 11: 77920832 | 57384 | 11: 77922179 | 57385 | 11: 77922588 | 57386 | 11: 77923067 |
| 57387 | 11: 77927350 | 57398 | 11: 78226756 | 57401 | 11: 78268974 | 57402 | 11: 78275050 | 57403 | 11: 78303645 |
| 57407 | 11: 78375724 | 57412 | 11: 78529857 | 57415 | 11: 78631628 | 57417 | 11: 78663074 | 57419 | 11: 78724728 |
| 57430 | 11: 78997006 | 57438 | 11: 79084041 | 57452 | 11: 79229476 | 57454 | 11: 79233862 | 57479 | 11: 79764049 |
| 57489 | 11: 80152318 | 57494 | 11: 80309473 | 57496 | 11: 80321806 | 57505 | 11: 80474151 | 57507 | 11: 80513212 |
| 57514 | 11: 80648310 | 57515 | 11: 80671943 | 57516 | 11: 80690342 | 57545 | 11: 81671201 | 57546 | 11: 81671227 |
| 57551 | 11: 81785970 | 57552 | 11: 81800692 | 57558 | 11: 81881809 | 57575 | 11: 82260811 | 57580 | 11: 82351733 |
| 57583 | 11: 82364482 | 57591 | 11: 82664554 | 57592 | 11: 82664600 | 57593 | 11: 82664609 | 57594 | 11: 82664725 |
| 57596 | 11: 82671761 | 57597 | 11: 82677475 | 57598 | 11: 82677728 | 57599 | 11: 82683785 | 57600 | 11: 82686842 |
| 57601 | 11: 82687013 | 57602 | 11: 82687072 | 57603 | 11: 82687666 | 57604 | 11: 82700336 | 57605 | 11: 82709413 |
| 57621 | 11: 83006401 | 57622 | 11: 83027036 | 57623 | 11: 83038638 | 57629 | 11: 83118274 | 57630 | 11: 83137062 |
| 57652 | 11: 83676352 | 57692 | 11: 84965273 | 57709 | 11: 85278395 | 57711 | 11: 85316120 | 57712 | 11: 85350978 |
| 57720 | 11: 85498225 | 57722 | 11: 85595081 | 57731 | 11: 85746062 | 57733 | 11: 85771451 | 57736 | 11: 85853743 |
| 57738 | 11: 85936758 | 57748 | 11: 86094641 | 57750 | 11: 86113594 | 57753 | 11: 86201573 | 57757 | 11: 86221979 |
| 57758 | 11: 86224683 | 57789 | 11: 87198869 | 57790 | 11: 87208683 | 57792 | 11: 87231125 | 57797 | 11: 87357375 |
| 57798 | 11: 87380616 | 57803 | 11: 87492469 | 57804 | 11: 87494268 | 57805 | 11: 87495906 | 57807 | 11: 87520617 |
| 57808 | 11: 87530939 | 57812 | 11: 87650800 | 57815 | 11: 87710317 | 57821 | 11: 87813732 | 57823 | 11: 87882552 |
| 57829 | 11: 87970096 | 57830 | 11: 87972654 | 57831 | 11: 87972763 | 57832 | 11: 87975372 | 57833 | 11: 87980700 |
| 57834 | 11: 87982062 | 57835 | 11: 87982864 | 57836 | 11: 87986573 | 57837 | 11: 87987392 | 57838 | 11: 87991576 |
| 57839 | 11: 87992353 | 57840 | 11: 87994077 | 57841 | 11: 87994761 | 57842 | 11: 87995976 | 57843 | 11: 87996439 |
| 57844 | 11: 87997041 | 57845 | 11: 87997518 | 57846 | 11: 87998024 | 57848 | 11: 88000459 | 57849 | 11: 88001097 |
| 57850 | 11: 88002138 | 57851 | 11: 88003290 | 57852 | 11: 88006051 | 57853 | 11: 88006580 | 57854 | 11: 88007139 |
| 57855 | 11: 88008201 | 57857 | 11: 88009081 | 57858 | 11: 88009883 | 57859 | 11: 88011349 | 57860 | 11: 88013047 |
| 57862 | 11: 88020282 | 57863 | 11: 88021880 | 57864 | 11: 88030641 | 57866 | 11: 88045066 | 57873 | 11: 88105264 |
| 57885 | 11: 88479108 | 57890 | 11: 88733341 | 57897 | 11: 88922785 | 57898 | 11: 88922827 | 57899 | 11: 88922860 |
| 57902 | 11: 88976021 | 57904 | 11: 88994635 | 57909 | 11: 89132552 | 57910 | 11: 89133642 | 57911 | 11: 89152187 |
| 57912 | 11: 89213759 | 57919 | 11: 89375784 | 57920 | 11: 89387726 | 57929 | 11: 89561970 | 57940 | 11: 89843767 |
| 57960 | 11: 90408075 | 57965 | 11: 90443507 | 57970 | 11: 90503652 | 57977 | 11: 90744044 | 57979 | 11: 90785269 |
| 57980 | 11: 90787434 | 57984 | 11: 90906061 | 57989 | 11: 90982703 | 57991 | 11: 91054916 | 57994 | 11: 91084013 |
| 58002 | 11: 91266370 | 58006 | 11: 91343800 | 58061 | 11: 92402829 | 58062 | 11: 92454234 | 58063 | 11: 92473780 |
| 58064 | 11: 92476182 | 58073 | 11: 92661797 | 58074 | 11: 92674766 | 58077 | 11: 92715331 | 58084 | 11: 92833095 |
| 58095 | 11: 93210615 | 58098 | 11: 93223406 | 58116 | 11: 93629686 | 58119 | 11: 93727304 | 58126 | 11: 93972515 |
| 58127 | 11: 93973132 | 58128 | 11: 93973249 | 58129 | 11: 93973432 | 58131 | 11: 94067508 | 58132 | 11: 94076855 |
| 58137 | 11: 94178079 | 58140 | 11: 94211102 | 58144 | 11: 94339459 | 58148 | 11: 94453289 | 58151 | 11: 94500564 |
| 58152 | 11: 94531181 | 58164 | 11: 94880948 | 58173 | 11: 95280882 | 58177 | 11: 95315895 | 58178 | 11: 95322263 |
| 58181 | 11: 95425616 | 58190 | 11: 95586651 | 58193 | 11: 95692063 | 58201 | 11: 95840093 | 58207 | 11: 96117932 |
| 58209 | 11: 96142116 | 58213 | 11: 96261764 | 58215 | 11: 96285921 | 58216 | 11: 96322123 | 58217 | 11: 96322228 |
| 58218 | 11: 96338458 | 58230 | 11: 96820150 | 58245 | 11: 97196967 | 58248 | 11: 97225356 | 58251 | 11: 97254785 |
| 58253 | 11: 97286893 | 58276 | 11: 97843712 | 58277 | 11: 97843818 | 58278 | 11: 97883599 | 58279 | 11: 97892319 |
| 58289 | 11: 98162222 | 58290 | 11: 98162477 | 58291 | 11: 98173675 | 58292 | 11: 98179996 | 58293 | 11: 98189444 |
| 58294 | 11: 98191816 | 58295 | 11: 98193416 | 58298 | 11: 98222190 | 58304 | 11: 98396073 | 58313 | 11: 98726635 |
| 58316 | 11: 98813314 | 58321 | 11: 98866430 | 58327 | 11: 98921444 | 58328 | 11: 98921481 | 58331 | 11: 98929180 |
| 58332 | 11: 98929534 | 58333 | 11: 98931991 | 58334 | 11: 98932587 | 58335 | 11: 98951509 | 58337 | 11: 98980770 |
| 58338 | 11: 99034896 | 58339 | 11: 99114771 | 58342 | 11: 99223633 | 58343 | 11: 99225289 | 58346 | 11: 99292200 |
| 58347 | 11: 99366886 | 58348 | 11: 99391843 | 58349 | 11: 99395118 | 58350 | 11: 99395609 | 58351 | 11: 99396169 |
| 58352 | 11: 99397143 | 58353 | 11: 99398436 | 58354 | 11: 99399218 | 58355 | 11: 99400709 | 58356 | 11: 99401571 |
| 58357 | 11: 99402294 | 58358 | 11: 99403416 | 58359 | 11: 99409144 | 58360 | 11: 99424233 | 58361 | 11: 99434036 |
| 58362 | 11: 99444575 | 58368 | 11: 99668885 | 58378 | 11: 99888002 | 58382 | 11: 99923257 | 58383 | 11: 99932292 |
| 58384 | 11: 99932294 | 58392 | 11: 10002865 | 58402 | 11: 10020523 | 58404 | 11: 10023875 | 58405 | 11: 10024462 |
| 58408 | 11: 10034694 | 58413 | 11: 10046250 | 58421 | 11: 10065868 | 58423 | 11: 10069663 | 58432 | 11: 10089413 |
| 58433 | 11: 10089414 | 58434 | 11: 10089418 | 58435 | 11: 10089420 | 58436 | 11: 10089422 | 58437 | 11: 10089423 |
| 58438 | 11: 10089584 | 58439 | 11: 10089862 | 58440 | 11: 10089910 | 58441 | 11: 10090030 | 58442 | 11: 10090083 |
| 58443 | 11: 10090139 | 58444 | 11: 10090298 | 58445 | 11: 10090301 | 58446 | 11: 10090303 | 58447 | 11: 10090310 |
| 58448 | 11: 10090437 | 58449 | 11: 10090548 | 58450 | 11: 10090552 | 58451 | 11: 10090559 | 58452 | 11: 10092003 |
| 58453 | 11: 10092018 | 58455 | 11: 10092948 | 58456 | 11: 10092991 | 58465 | 11: 10121113 | 58469 | 11: 10139576 |
| 58481 | 11: 10176766 | 58482 | 11: 10179134 | 58490 | 11: 10195025 | 58498 | 11: 10216529 | 58503 | 11: 10235669 |
| 58505 | 11: 10236915 | 58507 | 11: 10245112 | 58508 | 11: 10245553 | 58509 | 11: 10245561 | 58510 | 11: 10245564 |
| 58512 | 11: 10248040 | 58520 | 11: 10260314 | 58521 | 11: 10260318 | 58527 | 11: 10273417 | 58534 | 11: 10280891 |
| 58535 | 11: 10282294 | 58537 | 11: 10284704 | 58539 | 11: 10287469 | 58540 | 11: 10287825 | 58541 | 11: 10287894 |
| 58542 | 11: 10288318 | 58543 | 11: 10288773 | 58544 | 11: 10289321 | 58545 | 11: 10289432 | 58546 | 11: 10289512 |
| 58547 | 11: 10289609 | 58548 | 11: 10289667 | 58549 | 11: 10289886 | 58550 | 11: 10289974 | 58551 | 11: 10290070 |
| 58552 | 11: 10290097 | 58553 | 11: 10290321 | 58555 | 11: 10293724 | 58561 | 11: 10303129 | 58563 | 11: 10311374 |
| 58565 | 11: 10312784 | 58570 | 11: 10325911 | 58571 | 11: 10325913 | 58572 | 11: 10325914 | 58573 | 11: 10325923 |
| 58574 | 11: 10327300 | 58580 | 11: 10337533 | 58582 | 11: 10338027 | 58586 | 11: 10342689 | 58599 | 11: 10376543 |
| 58603 | 11: 10389419 | 58610 | 11: 10416326 | 58611 | 11: 10416332 | 58614 | 11: 10418835 | 58616 | 11: 10420735 |
| 58621 | 11: 10424706 | 58626 | 11: 10440574 | 58641 | 11: 10466512 | 58647 | 11: 10471723 | 58648 | 11: 10472240 |
| 58664 | 11: 10499756 | 58666 | 11: 10508228 | 58669 | 11: 10514921 | 58675 | 11: 10524663 | 58676 | 11: 10526769 |
| 58678 | 11: 10536852 | 58679 | 11: 10536862 | 58682 | 11: 10549667 | 58683 | 11: 10556546 | 58684 | 11: 10556548 |
| 58685 | 11: 10556557 | 58686 | 11: 10558647 | 58687 | 11: 10559874 | 58688 | 11: 10560239 | 58689 | 11: 10560299 |
| 58690 | 11: 10560406 | 58691 | 11: 10560652 | 58692 | 11: 10560775 | 58693 | 11: 10560879 | 58694 | 11: 10560986 |
| 58695 | 11: 10561194 | 58696 | 11: 10561355 | 58697 | 11: 10561585 | 58698 | 11: 10561748 | 58699 | 11: 10561828 |
| 58700 | 11: 10562085 | 58701 | 11: 10562158 | 58702 | 11: 10562413 | 58703 | 11: 10562929 | 58704 | 11: 10567074 |
| 58705 | 11: 10569241 | 58707 | 11: 10576176 | 58712 | 11: 10606865 | 58713 | 11: 10608376 | 58715 | 11: 10621054 |
| 58717 | 11: 10634575 | 58719 | 11: 10644949 | 58722 | 11: 10657444 | 58723 | 11: 10657459 | 58724 | 11: 10657485 |
| 58725 | 11: 10661179 | 58726 | 11: 10661476 | 58727 | 11: 10662633 | 58740 | 12: 317570 | 58748 | 12: 542288 |
| 58757 | 12: 877215 | 58797 | 12: 1923479 | 58805 | 12: 2038461 | 58813 | 12: 2256922 | 58819 | 12: 2323582 |
| 58825 | 12: 2448377 | 58826 | 12: 2448435 | 58827 | 12: 2452533 | 58828 | 12: 2452685 | 58829 | 12: 2509817 |
| 58836 | 12: 2603168 | 58839 | 12: 2644727 | 58840 | 12: 2644772 | 58841 | 12: 2644793 | 58844 | 12: 2731342 |
| 58846 | 12: 2771593 | 58923 | 12: 4734740 | 58937 | 12: 5055337 | 59006 | 12: 7477665 | 59040 | 12: 8789258 |
| 59041 | 12: 8796877 | 59053 | 12: 9132925 | 59062 | 12: 9382289 | 59066 | 12: 9448144 | 59077 | 12: 9708609 |
| 59078 | 12: 9708626 | 59093 | 12: 10135415 | 59095 | 12: 10160043 | 59114 | 12: 10462955 | 59122 | 12: 10621365 |
| 59123 | 12: 10622131 | 59124 | 12: 10631694 | 59125 | 12: 10631700 | 59134 | 12: 10781199 | 59152 | 12: 11193032 |
| 59170 | 12: 11622983 | 59171 | 12: 11641143 | 59173 | 12: 11670644 | 59175 | 12: 11684828 | 59186 | 12: 11878651 |
| 59189 | 12: 11933919 | 59193 | 12: 12026687 | 59195 | 12: 12066263 | 59196 | 12: 12069687 | 59197 | 12: 12087862 |
| 59198 | 12: 12123346 | 59206 | 12: 12322089 | 59210 | 12: 12404824 | 59219 | 12: 12760273 | 59224 | 12: 12974557 |
| 59231 | 12: 13161754 | 59239 | 12: 13388932 | 59241 | 12: 13411406 | 59242 | 12: 13473520 | 59243 | 12: 13518818 |
| 59246 | 12: 13578439 | 59247 | 12: 13591408 | 59250 | 12: 13656437 | 59261 | 12: 13824057 | 59268 | 12: 13927173 |
| 59280 | 12: 14131140 | 59281 | 12: 14132377 | 59283 | 12: 14157283 | 59285 | 12: 14211580 | 59286 | 12: 14211590 |
| 59287 | 12: 14211610 | 59288 | 12: 14240924 | 59292 | 12: 14361824 | 59293 | 12: 14372218 | 59294 | 12: 14388145 |
| 59295 | 12: 14399933 | 59299 | 12: 14456910 | 59302 | 12: 14520853 | 59306 | 12: 14595181 | 59311 | 12: 14716329 |
| 59319 | 12: 14988572 | 59321 | 12: 15005389 | 59324 | 12: 15101552 | 59333 | 12: 15266724 | 59335 | 12: 15281976 |
| 59344 | 12: 15465428 | 59348 | 12: 15534714 | 59350 | 12: 15552667 | 59352 | 12: 15567714 | 59353 | 12: 15577383 |
| 59355 | 12: 15618825 | 59360 | 12: 15729746 | 59363 | 12: 15770061 | 59366 | 12: 15810335 | 59373 | 12: 15938324 |
| 59380 | 12: 16125597 | 59381 | 12: 16136554 | 59386 | 12: 16274543 | 59390 | 12: 16346416 | 59394 | 12: 16413245 |
| 59410 | 12: 16793613 | 59414 | 12: 16900417 | 59423 | 12: 17162528 | 59425 | 12: 17199573 | 59430 | 12: 17324217 |
| 59435 | 12: 17368614 | 59440 | 12: 17470036 | 59453 | 12: 17926477 | 59459 | 12: 18040185 | 59468 | 12: 18229565 |
| 59469 | 12: 18253619 | 59474 | 12: 18390032 | 59475 | 12: 18391314 | 59486 | 12: 18697557 | 59492 | 12: 18898137 |
| 59496 | 12: 18919039 | 59507 | 12: 19071459 | 59509 | 12: 19100832 | 59517 | 12: 19186475 | 59519 | 12: 19221055 |
| 59520 | 12: 19233240 | 59522 | 12: 19351299 | 59523 | 12: 19367974 | 59524 | 12: 19367977 | 59528 | 12: 19460445 |
| 59545 | 12: 19931195 | 59550 | 12: 20111148 | 59551 | 12: 20111169 | 59578 | 12: 20706397 | 59582 | 12: 20914485 |
| 59588 | 12: 21042616 | 59591 | 12: 21070776 | 59597 | 12: 21212930 | 59600 | 12: 21309091 | 59602 | 12: 21368915 |
| 59613 | 12: 21811288 | 59617 | 12: 21924603 | 59618 | 12: 21936752 | 59620 | 12: 21945571 | 59621 | 12: 21969243 |
| 59627 | 12: 22146118 | 59631 | 12: 22263714 | 59642 | 12: 22573355 | 59643 | 12: 22576349 | 59647 | 12: 22662928 |
| 59653 | 12: 22821830 | 59654 | 12: 22834458 | 59663 | 12: 23060365 | 59667 | 12: 23201662 | 59671 | 12: 23249052 |
| 59677 | 12: 23371031 | 59680 | 12: 23439101 | 59696 | 12: 23931995 | 59703 | 12: 24076421 | 59710 | 12: 24256758 |
| 59716 | 12: 24331867 | 59721 | 12: 24471307 | 59733 | 12: 24731505 | 59734 | 12: 24748284 | 59737 | 12: 24838866 |
| 59741 | 12: 24922215 | 59742 | 12: 24925957 | 59747 | 12: 25022781 | 59749 | 12: 25034947 | 59754 | 12: 25118490 |
| 59766 | 12: 25470724 | 59767 | 12: 25498467 | 59769 | 12: 25589790 | 59777 | 12: 25724323 | 59798 | 12: 26442722 |
| 59805 | 12: 26653552 | 59808 | 12: 26751983 | 59814 | 12: 26874891 | 59820 | 12: 26979789 | 59821 | 12: 26986008 |
| 59830 | 12: 27189356 | 59841 | 12: 27380634 | 59848 | 12: 27503259 | 59850 | 12: 27528591 | 59854 | 12: 27721180 |
| 59855 | 12: 27723132 | 59856 | 12: 27723229 | 59861 | 12: 27998192 | 59862 | 12: 28002030 | 59878 | 12: 28526148 |
| 59879 | 12: 28587383 | 59881 | 12: 28621183 | 59893 | 12: 28989343 | 59913 | 12: 29539899 | 59934 | 12: 30141838 |
| 59944 | 12: 30381579 | 59945 | 12: 30411312 | 59947 | 12: 30444618 | 59949 | 12: 30463422 | 59969 | 12: 30909596 |
| 59971 | 12: 30979325 | 59973 | 12: 30983862 | 59974 | 12: 30991739 | 59977 | 12: 31053899 | 59979 | 12: 31059835 |
| 59980 | 12: 31115741 | 59984 | 12: 31247300 | 59985 | 12: 31247421 | 59986 | 12: 31247454 | 59987 | 12: 31247486 |
| 59998 | 12: 31454833 | 60007 | 12: 31767190 | 60011 | 12: 31790778 | 60012 | 12: 31797116 | 60018 | 12: 31950273 |
| 60023 | 12: 32162560 | 60028 | 12: 32295933 | 60029 | 12: 32338936 | 60032 | 12: 32379592 | 60033 | 12: 32387590 |
| 60034 | 12: 32391405 | 60037 | 12: 32450230 | 60038 | 12: 32450232 | 60040 | 12: 32487563 | 60058 | 12: 33054434 |
| 60061 | 12: 33082346 | 60062 | 12: 33085573 | 60066 | 12: 33139330 | 60075 | 12: 33340233 | 60086 | 12: 33674224 |
| 60095 | 12: 33970418 | 60096 | 12: 34054782 | 60097 | 12: 34068458 | 60109 | 12: 34500254 | 60114 | 12: 34681917 |
| 60126 | 12: 34900938 | 60133 | 12: 35115665 | 60140 | 12: 35310855 | 60150 | 12: 35346851 | 60164 | 12: 35747252 |
| 60173 | 12: 36216803 | 60175 | 12: 36341507 | 60176 | 12: 36361684 | 60178 | 12: 36421378 | 60179 | 12: 36443014 |
| 60181 | 12: 36474506 | 60182 | 12: 36483162 | 60183 | 12: 36486137 | 60184 | 12: 36504213 | 60189 | 12: 36706435 |
| 60191 | 12: 36753159 | 60195 | 12: 36952382 | 60196 | 12: 36952416 | 60210 | 12: 37329971 | 60211 | 12: 37330062 |
| 60212 | 12: 37330099 | 60216 | 12: 37420551 | 60256 | 12: 39391630 | 60260 | 12: 39553608 | 60267 | 12: 39684127 |
| 60270 | 12: 39776454 | 60279 | 12: 40049870 | 60281 | 12: 40141911 | 60311 | 12: 41076939 | 60329 | 12: 41861946 |
| 60332 | 12: 41912763 | 60402 | 12: 44937336 | 60404 | 12: 44979318 | 60422 | 12: 45617161 | 60442 | 12: 46329415 |
| 60445 | 12: 46430991 | 60466 | 12: 46961986 | 60470 | 12: 47018801 | 60474 | 12: 47110627 | 60484 | 12: 47309976 |
| 60487 | 12: 47407515 | 60508 | 12: 48047961 | 60510 | 12: 48150915 | 60538 | 12: 48960299 | 60549 | 12: 49199679 |
| 60552 | 12: 49236831 | 60566 | 12: 49561422 | 60571 | 12: 49723456 | 60574 | 12: 49789492 | 60579 | 12: 49873319 |
| 60580 | 12: 49881472 | 60584 | 12: 49919596 | 60600 | 12: 50189626 | 60609 | 12: 50384404 | 60611 | 12: 50424103 |
| 60612 | 12: 50439814 | 60614 | 12: 50497707 | 60615 | 12: 50517145 | 60616 | 12: 50525554 | 60617 | 12: 50530876 |
| 60619 | 12: 50579721 | 60628 | 12: 50806532 | 60630 | 12: 50840958 | 60632 | 12: 50869818 | 60633 | 12: 50871829 |
| 60660 | 12: 51551817 | 60661 | 12: 51626384 | 60679 | 12: 52103992 | 60687 | 12: 52262646 | 60692 | 12: 52346386 |
| 60702 | 12: 52660295 | 60710 | 12: 52806268 | 60727 | 12: 53053258 | 60729 | 12: 53061165 | 60738 | 12: 53338165 |
| 60739 | 12: 53339046 | 60740 | 12: 53339054 | 60749 | 12: 53523285 | 60750 | 12: 53553256 | 60752 | 12: 53587669 |
| 60755 | 12: 53627763 | 60756 | 12: 53640047 | 60763 | 12: 53801258 | 60781 | 12: 54131348 | 60782 | 12: 54153871 |
| 60797 | 12: 54494995 | 60803 | 12: 54630943 | 60804 | 12: 54634286 | 60810 | 12: 54736016 | 60813 | 12: 54833257 |
| 60815 | 12: 54927343 | 60819 | 12: 55024877 | 60821 | 12: 55068443 | 60848 | 12: 55943119 | 60851 | 12: 56039366 |
| 60880 | 12: 56976868 | 60882 | 12: 57050432 | 60904 | 12: 57798387 | 60905 | 12: 57798425 | 60912 | 12: 57952881 |
| 60914 | 12: 58028126 | 60929 | 12: 58672694 | 60957 | 12: 59417372 | 60969 | 12: 59853749 | 60978 | 12: 60236147 |
| 60990 | 12: 60736444 | 61018 | 12: 61855777 | 61024 | 12: 62071314 | 61028 | 12: 62095959 | 61031 | 12: 62215048 |
| 61034 | 12: 62291744 | 61035 | 12: 62297558 | 61051 | 12: 62665927 | 61053 | 12: 62697756 | 61065 | 12: 63053238 |
| 61080 | 12: 63589109 | 61085 | 12: 63730818 | 61089 | 12: 63774437 | 61094 | 12: 63920843 | 61100 | 12: 64086485 |
| 61112 | 12: 64454937 | 61126 | 12: 64874897 | 61127 | 12: 64896393 | 61148 | 12: 65376279 | 61151 | 12: 65433100 |
| 61156 | 12: 65616303 | 61168 | 12: 66123917 | 61170 | 12: 66204351 | 61175 | 12: 66340744 | 61199 | 12: 66896772 |
| 61206 | 12: 67184352 | 61207 | 12: 67228835 | 61210 | 12: 67289617 | 61216 | 12: 67417050 | 61231 | 12: 67757020 |
| 61237 | 12: 67889485 | 61252 | 12: 68144427 | 61255 | 12: 68171444 | 61256 | 12: 68171491 | 61262 | 12: 68316664 |
| 61274 | 12: 68605490 | 61280 | 12: 68760089 | 61283 | 12: 68784416 | 61289 | 12: 68861830 | 61321 | 12: 69512279 |
| 61322 | 12: 69512294 | 61323 | 12: 69516800 | 61331 | 12: 69577020 | 61338 | 12: 69681798 | 61348 | 12: 70147062 |
| 61367 | 12: 71314524 | 61383 | 12: 71639973 | 61386 | 12: 71790421 | 61390 | 12: 71842435 | 61408 | 12: 72931638 |
| 61414 | 12: 73012734 | 61421 | 12: 73132371 | 61422 | 12: 73154971 | 61427 | 12: 73317927 | 61428 | 12: 73324264 |
| 61441 | 12: 73504167 | 61443 | 12: 73515714 | 61444 | 12: 73515717 | 61445 | 12: 73515812 | 61454 | 12: 73622472 |
| 61455 | 12: 73637550 | 61462 | 12: 73750586 | 61469 | 12: 73899680 | 61475 | 12: 74054821 | 61486 | 12: 74328198 |
| 61495 | 12: 74492364 | 61498 | 12: 74505038 | 61500 | 12: 74526665 | 61517 | 12: 74949213 | 61530 | 12: 75243236 |
| 61535 | 12: 75318404 | 61538 | 12: 75375959 | 61542 | 12: 75403639 | 61543 | 12: 75471804 | 61558 | 12: 75839630 |
| 61567 | 12: 76074190 | 61569 | 12: 76110122 | 61581 | 12: 76477335 | 61587 | 12: 76644169 | 61588 | 12: 76649683 |
| 61605 | 12: 77139238 | 61609 | 12: 77240177 | 61627 | 12: 77604409 | 61632 | 12: 77695108 | 61641 | 12: 77891817 |
| 61644 | 12: 77992115 | 61647 | 12: 78041886 | 61655 | 12: 78246588 | 61669 | 12: 78577349 | 61691 | 12: 79095439 |
| 61692 | 12: 79098661 | 61693 | 12: 79098791 | 61694 | 12: 79099207 | 61695 | 12: 79099246 | 61696 | 12: 79104872 |
| 61710 | 12: 79403358 | 61715 | 12: 79476337 | 61727 | 12: 79736085 | 61740 | 12: 79970178 | 61756 | 12: 80319360 |
| 61775 | 12: 80804698 | 61790 | 12: 81214960 | 61791 | 12: 81215027 | 61803 | 12: 81507963 | 61804 | 12: 81508005 |
| 61819 | 12: 81781296 | 61820 | 12: 81840070 | 61824 | 12: 81902349 | 61840 | 12: 82263825 | 61845 | 12: 82445861 |
| 61848 | 12: 82510342 | 61877 | 12: 83071777 | 61882 | 12: 83242885 | 61891 | 12: 83362092 | 61907 | 12: 83699595 |
| 61915 | 12: 83925298 | 61920 | 12: 84006533 | 61926 | 12: 84137136 | 61945 | 12: 84691330 | 61946 | 12: 84709160 |
| 61948 | 12: 84744945 | 61949 | 12: 84744950 | 61955 | 12: 84883821 | 61960 | 12: 84929565 | 61966 | 12: 84993056 |
| 61969 | 12: 85038979 | 61970 | 12: 85051267 | 61973 | 12: 85122781 | 61978 | 12: 85226982 | 61979 | 12: 85230036 |
| 61981 | 12: 85245683 | 61983 | 12: 85268459 | 61984 | 12: 85293670 | 61986 | 12: 85321955 | 61988 | 12: 85328690 |
| 61990 | 12: 85352315 | 61991 | 12: 85354082 | 61994 | 12: 85362756 | 61995 | 12: 85375388 | 61997 | 12: 85396298 |
| 62000 | 12: 85462507 | 62007 | 12: 85632578 | 62013 | 12: 85790348 | 62014 | 12: 85790349 | 62035 | 12: 86313847 |
| 62038 | 12: 86341413 | 62043 | 12: 86507763 | 62045 | 12: 86516372 | 62046 | 12: 86516421 | 62048 | 12: 86519761 |
| 62049 | 12: 86565634 | 52051 | 12: 86656646 | 62052 | 12: 86687289 | 62053 | 12: 86688052 | 62056 | 12: 86734179 |
| 62058 | 12: 86744155 | 62059 | 12: 86764063 | 62062 | 12: 86796745 | 62064 | 12: 86824616 | 62068 | 12: 86895391 |
| 62069 | 12: 86895562 | 62070 | 12: 86897090 | 62071 | 12: 86897178 | 62072 | 12: 86901814 | 62073 | 12: 86901834 |
| 62074 | 12: 86901846 | 62075 | 12: 86901945 | 62076 | 12: 86902939 | 62077 | 12: 86902946 | 62078 | 12: 86907527 |
| 62079 | 12: 86907549 | 62080 | 12: 86908054 | 62082 | 12: 86924401 | 62085 | 12: 86976449 | 62086 | 12: 86988484 |
| 62089 | 12: 87019731 | 62090 | 12: 87024506 | 62095 | 12: 87094553 | 62109 | 13: 837989 | 62110 | 13: 859954 |
| 62115 | 13: 877219 | 62122 | 13: 1025761 | 62126 | 13: 1174223 | 62127 | 13: 1184941 | 62138 | 13: 1435091 |
| 62143 | 13: 1589719 | 62145 | 13: 1615667 | 62154 | 13: 1741335 | 62155 | 13: 1754933 | 62165 | 13: 2014401 |
| 62169 | 13: 2079335 | 62174 | 13: 2108318 | 62176 | 13: 2133989 | 62181 | 13: 2232512 | 62188 | 13: 2272732 |
| 62193 | 13: 2391867 | 62194 | 13: 2394109 | 62195 | 13: 2400920 | 62196 | 13: 2402378 | 62200 | 13: 2507817 |
| 62203 | 13: 2551762 | 62208 | 13: 2625076 | 62209 | 13: 2632788 | 62238 | 13: 3279851 | 62250 | 13: 3588425 |
| 62254 | 13: 3701619 | 62259 | 13: 3860445 | 62265 | 13: 3974443 | 62270 | 13: 4084139 | 62271 | 13: 4084171 |
| 62272 | 13: 4084181 | 62276 | 13: 4221428 | 62278 | 13: 4302438 | 62291 | 13: 4792202 | 62292 | 13: 4793338 |
| 62312 | 13: 5324308 | 62314 | 13: 5360261 | 62325 | 13: 5525194 | 62326 | 13: 5525225 | 62327 | 13: 5525250 |
| 62341 | 13: 5742073 | 62356 | 13: 6112364 | 62377 | 13: 6686807 | 62379 | 13: 6694933 | 62384 | 13: 6837817 |
| 62392 | 13: 7036389 | 62399 | 13: 7224958 | 62406 | 13: 7441639 | 62412 | 13: 7553513 | 62425 | 13: 7848557 |
| 62441 | 13: 8487756 | 62465 | 13: 8980654 | 62477 | 13: 9357735 | 62486 | 13: 9550333 | 62487 | 13: 9576478 |
| 62496 | 13: 9729764 | 62497 | 13: 9729772 | 62512 | 13: 10026414 | 62514 | 13: 10070381 | 62522 | 13: 10306871 |
| 62523 | 13: 10395756 | 62529 | 13: 10478767 | 62533 | 13: 10517565 | 62542 | 13: 10720404 | 62543 | 13: 10724228 |
| 62547 | 13: 10815809 | 62549 | 13: 10956279 | 62550 | 13: 10956346 | 62551 | 13: 10956354 | 62552 | 13: 10956355 |
| 62559 | 13: 11536433 | 62561 | 13: 11561251 | 62562 | 13: 11598409 | 62566 | 13: 11792199 | 62568 | 13: 11904569 |
| 62581 | 13: 12520500 | 62584 | 13: 12616676 | 62585 | 13: 12669714 | 62593 | 13: 12825549 | 62597 | 13: 12901657 |
| 62601 | 13: 12957260 | 62604 | 13: 13014675 | 62606 | 13: 13050532 | 62609 | 13: 13134686 | 62620 | 13: 13394218 |
| 62624 | 13: 13416442 | 62629 | 13: 13525172 | 62633 | 13: 13724404 | 62648 | 13: 14180541 | 62709 | 13: 15922152 |
| 62710 | 13: 15926948 | 62713 | 13: 16113457 | 62714 | 13: 16141551 | 62715 | 13: 16161619 | 62716 | 13: 16198035 |
| 62727 | 13: 16479721 | 62752 | 13: 16821257 | 62753 | 13: 16838893 | 62758 | 13: 16979210 | 62766 | 13: 17176766 |
| 62775 | 13: 17311025 | 62776 | 13: 17311026 | 62777 | 13: 17311048 | 62778 | 13: 17311402 | 62779 | 13: 17313556 |
| 62780 | 13: 17321384 | 62781 | 13: 17327212 | 62784 | 13: 17371815 | 62791 | 13: 17532109 | 62792 | 13: 17537709 |
| 62793 | 13: 17537777 | 62794 | 13: 17537795 | 62795 | 13: 17540260 | 62796 | 13: 17540346 | 62797 | 13: 17543085 |
| 62800 | 13: 17568204 | 62804 | 13: 17707891 | 62805 | 13: 17739721 | 62807 | 13: 17780066 | 62809 | 13: 17793717 |
| 62810 | 13: 17796108 | 62811 | 13: 17797006 | 62812 | 13: 17797028 | 62813 | 13: 17797078 | 62814 | 13: 17800892 |
| 62819 | 13: 17959881 | 62821 | 13: 18000186 | 62822 | 13: 18000196 | 62823 | 13: 18001014 | 62824 | 13: 18001496 |
| 62825 | 13: 18001505 | 62826 | 13: 18001601 | 62828 | 13: 18117994 | 62831 | 13: 18160335 | 62843 | 13: 18626070 |
| 62847 | 13: 18666175 | 62853 | 13: 18721208 | 62881 | 13: 18755905 | 62883 | 13: 18792716 | 62884 | 13: 18792825 |
| 62885 | 13: 18792850 | 62889 | 13: 18828811 | 62891 | 13: 18892498 | 62892 | 13: 18897253 | 62894 | 13: 19005851 |
| 62895 | 13: 19009052 | 62896 | 13: 19009093 | 62897 | 13: 19009113 | 62902 | 13: 19106619 | 62911 | 13: 19331702 |
| 62917 | 13: 19457766 | 62919 | 13: 19497053 | 62920 | 13: 19497075 | 62921 | 13: 19497132 | 62922 | 13: 19497137 |
| 62923 | 13: 19539755 | 62924 | 13: 19567930 | 62926 | 13: 19588565 | 62929 | 13: 19655690 | 62930 | 13: 19688739 |
| 62933 | 13: 19726581 | 62935 | 13: 19733380 | 62941 | 13: 19822361 | 62942 | 13: 19829540 | 62948 | 13: 19908550 |
| 62950 | 13: 19929408 | 62951 | 13: 19985595 | 62966 | 13: 20376052 | 62973 | 13: 20455853 | 62974 | 13: 20455858 |
| 62975 | 13: 20455892 | 62976 | 13: 20455934 | 62977 | 13: 20455941 | 62986 | 13: 20690765 | 62989 | 13: 20711502 |
| 63003 | 13: 21079068 | 63004 | 13: 21080622 | 63005 | 13: 21080632 | 63006 | 13: 21080639 | 63007 | 13: 21080653 |
| 63008 | 13: 21080676 | 63009 | 13: 21080695 | 63015 | 13: 21196496 | 63021 | 13: 21338503 | 63025 | 13: 21463530 |
| 63033 | 13: 21602178 | 63034 | 13: 21608876 | 63036 | 13: 21617194 | 63037 | 13: 21669388 | 63038 | 13: 21673325 |
| 63046 | 13: 21871770 | 63053 | 13: 22005224 | 63067 | 13: 22364650 | 63069 | 13: 22400352 | 63075 | 13: 22556435 |
| 63083 | 13: 22843425 | 63085 | 13: 22943752 | 63094 | 13: 23364320 | 63095 | 13: 23428241 | 63100 | 13: 23590170 |
| 63107 | 13: 23799970 | 63113 | 13: 23993051 | 63122 | 13: 24204881 | 63125 | 13: 24378395 | 63128 | 13: 24432283 |
| 63129 | 13: 24464855 | 63138 | 13: 24550804 | 63144 | 13: 24721448 | 63148 | 13: 24785943 | 63152 | 13: 24889747 |
| 63157 | 13: 25026767 | 63162 | 13: 25139101 | 63163 | 13: 25157343 | 63164 | 13: 25169602 | 63171 | 13: 25314039 |
| 63176 | 13: 25373782 | 63177 | 13: 25408450 | 63178 | 13: 25410196 | 63185 | 13: 25553780 | 63186 | 13: 25556386 |
| 63197 | 13: 25892715 | 63200 | 13: 25911303 | 63201 | 13: 25911339 | 63202 | 13: 25911366 | 63204 | 13: 25958875 |
| 63207 | 13: 26100997 | 63216 | 13: 26381171 | 63217 | 13: 26385197 | 63218 | 13: 26403532 | 63228 | 13: 26709761 |
| 63229 | 13: 26798911 | 63246 | 13: 27211452 | 63249 | 13: 27252426 | 63255 | 13: 27371552 | 63259 | 13: 27465175 |
| 63260 | 13: 27465215 | 63261 | 13: 27465241 | 63262 | 13: 27465291 | 63264 | 13: 27517101 | 63267 | 13: 27585269 |
| 63269 | 13: 27634615 | 63274 | 13: 27760115 | 63275 | 13: 27831742 | 63276 | 13: 27837915 | 63277 | 13: 27868901 |
| 63278 | 13: 27871914 | 63279 | 13: 27897181 | 63280 | 13: 27902374 | 63281 | 13: 27904185 | 63293 | 13: 28285297 |
| 63298 | 13: 28416787 | 63306 | 13: 28593403 | 63307 | 13: 28631602 | 63309 | 13: 28668818 | 63317 | 13: 28768035 |
| 63322 | 13: 28950265 | 63330 | 13: 29182643 | 63337 | 13: 29330098 | 63339 | 13: 29336139 | 63350 | 13: 29535530 |
| 63353 | 13: 29588319 | 63357 | 13: 29751352 | 63369 | 13: 30024809 | 63371 | 13: 30056042 | 63375 | 13: 30104514 |
| 63376 | 13: 30183328 | 63377 | 13: 30183348 | 63378 | 13: 30194908 | 63379 | 13: 30194914 | 63380 | 13: 30194921 |
| 63381 | 13: 30194933 | 63382 | 13: 30195026 | 63383 | 13: 30195496 | 63385 | 13: 30210577 | 63388 | 13: 30242235 |
| 63408 | 13: 30707478 | 63410 | 13: 30754086 | 63418 | 13: 30976341 | 63422 | 13: 31020731 | 63437 | 13: 31276749 |
| 63438 | 13: 31276801 | 63440 | 13: 31276837 | 63441 | 13: 31280242 | 63442 | 13: 31280491 | 63443 | 13: 31282636 |
| 63444 | 13: 31282742 | 63445 | 13: 31282787 | 63446 | 13: 31285967 | 63447 | 13: 31285968 | 63448 | 13: 31285972 |
| 63449 | 13: 31286001 | 63450 | 13: 31286035 | 63451 | 13: 31296423 | 63456 | 13: 31420376 | 63459 | 13: 31447543 |
| 63466 | 13: 31582342 | 63471 | 13: 31736295 | 63488 | 13: 32202239 | 63491 | 13: 32240332 | 63496 | 13: 32324178 |
| 63497 | 13: 32362115 | 63499 | 13: 32365033 | 63527 | 13: 32808897 | 63538 | 13: 33049577 | 63540 | 13: 33078146 |
| 63545 | 13: 33129981 | 63547 | 13: 33172919 | 63549 | 13: 33261503 | 63554 | 13: 33326303 | 63561 | 13: 33610056 |
| 63562 | 13: 33610145 | 63563 | 13: 33615532 | 63564 | 13: 33641897 | 63566 | 13: 33682852 | 63569 | 13: 33817581 |
| 63570 | 13: 33818040 | 63573 | 13: 33900260 | 63584 | 13: 34538815 | 63586 | 13: 34570424 | 63587 | 13: 34574425 |
| 63588 | 13: 34595352 | 63592 | 13: 34690679 | 63594 | 13: 34725177 | 63596 | 13: 34746582 | 63597 | 13: 34750823 |
| 63598 | 13: 34798730 | 63603 | 13: 34909612 | 63607 | 13: 35010608 | 63608 | 13: 35019310 | 63610 | 13: 35093002 |
| 63613 | 13: 35138794 | 63616 | 13: 35156141 | 63620 | 13: 35193780 | 63624 | 13: 35229937 | 63626 | 13: 35230259 |
| 63629 | 13: 35259809 | 63630 | 13: 35273048 | 63632 | 13: 35319711 | 63674 | 13: 36409030 | 63675 | 13: 36424590 |
| 63680 | 13: 36570662 | 63682 | 13: 36589718 | 63687 | 13: 36656277 | 63691 | 13: 36716029 | 63694 | 13: 36827015 |
| 63696 | 13: 36854156 | 63720 | 13: 37443281 | 63732 | 13: 37745409 | 63742 | 13: 37991091 | 63757 | 13: 38329758 |
| 63758 | 13: 38375463 | 63760 | 13: 38420421 | 63762 | 13: 38501340 | 63776 | 13: 38828149 | 63777 | 13: 38828176 |
| 63778 | 13: 38828210 | 63779 | 13: 38828306 | 63780 | 13: 38828333 | 63784 | 13: 38935644 | 63787 | 13: 38957716 |
| 63806 | 13: 39270018 | 63808 | 13: 39276440 | 63813 | 13: 39359409 | 63814 | 13: 39360537 | 63817 | 13: 39403535 |
| 63821 | 13: 39464756 | 63822 | 13: 39498069 | 63824 | 13: 39559729 | 63825 | 13: 39574123 | 63826 | 13: 39584391 |
| 63828 | 13: 39644803 | 63834 | 13: 39704818 | 63847 | 13: 40027121 | 63901 | 13: 41141472 | 63926 | 13: 41826984 |
| 63928 | 13: 41862833 | 63929 | 13: 41863502 | 63944 | 13: 42197339 | 63952 | 13: 42439893 | 63956 | 13: 42526261 |
| 63961 | 13: 42557806 | 63963 | 13: 42567447 | 63964 | 13: 42570926 | 63965 | 13: 42576337 | 63966 | 13: 42578756 |
| 63967 | 13: 42579747 | 63968 | 13: 42581051 | 63969 | 13: 42583033 | 63970 | 13: 42585954 | 63971 | 13: 42587350 |
| 63973 | 13: 42588548 | 63974 | 13: 42589734 | 63975 | 13: 42590562 | 63976 | 13: 42591034 | 63977 | 13: 42591591 |
| 63978 | 13: 42592688 | 63979 | 13: 42602259 | 63980 | 13: 42604226 | 63981 | 13: 42605060 | 63982 | 13: 42606861 |
| 63983 | 13: 42607539 | 63984 | 13: 42617225 | 63985 | 13: 42619772 | 63986 | 13: 42620639 | 63987 | 13: 42622791 |
| 63988 | 13: 42624718 | 63990 | 13: 42628160 | 63991 | 13: 42632412 | 63992 | 13: 42636254 | 63994 | 13: 42655005 |
| 63995 | 13: 42655747 | 63997 | 13: 42707046 | 63998 | 13: 42733395 | 64000 | 13: 42775404 | 64002 | 13: 42816216 |
| 64010 | 13: 43060399 | 64013 | 13: 43174367 | 64017 | 13: 43269543 | 64018 | 13: 43342598 | 64019 | 13: 43400200 |
| 64021 | 13: 43503495 | 64030 | 13: 43617243 | 64034 | 13: 43735619 | 64035 | 13: 43736152 | 64061 | 13: 44136436 |
| 64064 | 13: 44159827 | 64066 | 13: 44231058 | 64070 | 13: 44319398 | 64073 | 13: 44364216 | 64079 | 13: 44475918 |
| 64080 | 13: 44491717 | 64091 | 13: 44736057 | 64116 | 13: 44978580 | 64117 | 13: 44978611 | 64119 | 13: 44989622 |
| 64128 | 13: 45018113 | 64129 | 13: 45022424 | 64130 | 13: 45034606 | 64132 | 13: 45038863 | 64133 | 13: 45046560 |
| 64135 | 13: 45057233 | 64136 | 13: 45081132 | 64138 | 13: 45099582 | 64139 | 13: 45108541 | 64140 | 13: 45110718 |
| 64141 | 13: 45112674 | 64142 | 13: 45114484 | 64144 | 13: 45129342 | 64145 | 13: 45135756 | 64146 | 13: 45143400 |
| 64147 | 13: 45144409 | 64149 | 13: 45160765 | 64173 | 13: 45837065 | 64188 | 13: 46248201 | 64189 | 13: 46260736 |
| 64190 | 13: 46262101 | 64191 | 13: 46280773 | 64192 | 13: 46303988 | 64193 | 13: 46308862 | 64197 | 13: 46342659 |
| 64203 | 13: 46433525 | 64205 | 13: 46541983 | 64217 | 13: 46759585 | 64219 | 13: 46767424 | 64222 | 13: 46855501 |
| 64226 | 13: 46934942 | 64230 | 13: 47057469 | 64235 | 13: 47187424 | 64240 | 13: 47305456 | 64242 | 13: 47333121 |
| 64243 | 13: 47346135 | 64265 | 13: 47860032 | 64267 | 13: 47916526 | 64268 | 13: 47927079 | 64270 | 13: 47952062 |
| 64271 | 13: 48003258 | 64272 | 13: 48017728 | 64279 | 13: 48210178 | 64281 | 13: 48212858 | 64313 | 13: 48933264 |
| 64327 | 13: 49369652 | 64371 | 13: 50683553 | 64377 | 13: 50955038 | 64379 | 13: 50985036 | 64385 | 13: 51191490 |
| 64388 | 13: 51268713 | 64389 | 13: 51269069 | 64404 | 13: 51645253 | 64408 | 13: 51745594 | 64412 | 13: 51889992 |
| 64413 | 13: 51917960 | 64424 | 13: 52280855 | 64425 | 13: 52333810 | 64430 | 13: 52514427 | 64436 | 13: 52666460 |
| 64443 | 13: 52794690 | 64444 | 13: 52797973 | 64445 | 13: 52798735 | 64446 | 13: 52798786 | 64448 | 13: 52806170 |
| 64450 | 13: 52838262 | 64457 | 13: 53001236 | 64461 | 13: 53139865 | 64463 | 13: 53146985 | 64465 | 13: 53149275 |
| 64468 | 13: 53184275 | 64470 | 13: 53189308 | 64471 | 13: 53191327 | 64472 | 13: 53192744 | 64473 | 13: 53193885 |
| 64474 | 13: 53195323 | 64475 | 13: 53197934 | 64476 | 13: 53199665 | 64477 | 13: 53200513 | 64478 | 13: 53201494 |
| 64479 | 13: 53205894 | 64480 | 13: 53211390 | 64481 | 13: 53212642 | 64482 | 13: 53213246 | 64483 | 13: 53214256 |
| 64484 | 13: 53221957 | 64485 | 13: 53223771 | 64486 | 13: 53225335 | 64490 | 13: 53267383 | 64491 | 13: 53287399 |
| 64502 | 13: 53656779 | 64503 | 13: 53668817 | 64521 | 13: 54501975 | 64524 | 13: 54544791 | 64528 | 13: 54637805 |
| 64532 | 13: 54688808 | 64539 | 13: 54867617 | 64540 | 13: 54924099 | 64541 | 13: 54930460 | 64542 | 13: 54963877 |
| 64545 | 13: 55001775 | 64546 | 13: 55009029 | 64549 | 13: 55107919 | 64551 | 13: 55137013 | 64553 | 13: 55164494 |
| 64556 | 13: 55256797 | 64558 | 13: 55286529 | 64559 | 13: 55306487 | 64563 | 13: 55363256 | 64576 | 13: 55652395 |
| 64579 | 13: 55736298 | 64586 | 13: 55924074 | 64596 | 13: 56129806 | 64599 | 13: 56206409 | 64602 | 13: 56257733 |
| 64613 | 13: 56553088 | 64615 | 13: 56567143 | 64616 | 13: 56574066 | 64628 | 13: 56987530 | 64632 | 13: 57050196 |
| 64642 | 13: 57391199 | 64643 | 13: 57394025 | 64654 | 13: 57698221 | 64655 | 13: 57736295 | 64656 | 13: 57744564 |
| 64657 | 13: 57744624 | 64664 | 13: 57962862 | 64677 | 13: 58287555 | 64678 | 13: 58288391 | 64679 | 13: 58297881 |
| 64683 | 13: 58337666 | 64688 | 13: 58408741 | 64689 | 13: 58453137 | 64690 | 13: 58475365 | 64693 | 13: 58521548 |
| 64700 | 13: 58687733 | 64703 | 13: 58755844 | 64705 | 13: 58823450 | 64709 | 13: 58925642 | 64711 | 13: 58935983 |
| 64713 | 13: 58968219 | 64718 | 13: 58981092 | 64737 | 13: 59317532 | 64738 | 13: 59317573 | 64739 | 13: 59317600 |
| 64752 | 13: 59798919 | 64753 | 13: 59810522 | 64757 | 13: 59881900 | 64763 | 13: 59982505 | 64765 | 13: 60004397 |
| 64775 | 13: 60191346 | 64777 | 13: 60219559 | 64779 | 13: 60249957 | 64781 | 13: 60270738 | 64782 | 13: 60273459 |
| 64786 | 13: 60360621 | 64787 | 13: 60363893 | 64803 | 13: 60828808 | 64813 | 13: 61235720 | 64819 | 13: 61487750 |
| 64823 | 13: 61526661 | 64824 | 13: 61536675 | 64830 | 13: 61748180 | 64833 | 13: 61837125 | 64857 | 13: 62340129 |
| 64864 | 13: 62437505 | 64865 | 13: 62437664 | 64869 | 13: 62616409 | 64870 | 13: 62619495 | 64871 | 13: 62620895 |
| 64872 | 13: 62620917 | 64873 | 13: 62623230 | 64874 | 13: 62623246 | 64875 | 13: 62623260 | 64876 | 13: 62623271 |
| 64877 | 13: 62623277 | 64878 | 13: 62623296 | 64879 | 13: 62623326 | 64890 | 13: 62758151 | 64895 | 13: 62805225 |
| 64896 | 13: 62808252 | 64898 | 13: 62825728 | 64903 | 13: 62901932 | 64904 | 13: 62913802 | 64905 | 13: 62957243 |
| 64909 | 13: 63100717 | 64910 | 13: 63113668 | 64911 | 13: 63115815 | 64912 | 13: 63117671 | 64915 | 13: 63231676 |
| 64916 | 13: 63238472 | 64922 | 13: 63381167 | 64924 | 13: 63406383 | 64927 | 13: 63465271 | 64932 | 13: 63632674 |
| 64933 | 13: 63667387 | 64936 | 13: 63800029 | 64941 | 13: 63926705 | 64944 | 13: 63945489 | 64945 | 13: 63950962 |
| 64964 | 13: 64427414 | 64970 | 13: 64579641 | 64973 | 13: 64608490 | 64979 | 13: 64765574 | 64981 | 13: 64780081 |
| 64985 | 13: 64917447 | 64992 | 13: 65197131 | 64993 | 13: 65222405 | 64994 | 13: 65226257 | 64996 | 13: 65300967 |
| 65002 | 13: 65421703 | 65003 | 13: 65421742 | 65008 | 13: 65551485 | 65009 | 13: 65556446 | 65012 | 13: 65615123 |
| 65013 | 13: 65633719 | 65015 | 13: 65678606 | 65016 | 13: 65710639 | 65017 | 13: 65711755 | 65018 | 13: 65713224 |
| 65019 | 13: 65713273 | 65020 | 13: 65714024 | 65022 | 13: 65714678 | 65024 | 13: 65734511 | 65025 | 13: 65737670 |
| 65026 | 13: 65747364 | 65027 | 13: 65751601 | 65028 | 13: 65757675 | 65029 | 13: 65768152 | 65030 | 13: 65769345 |
| 65031 | 13: 65771689 | 65032 | 13: 65778554 | 65038 | 13: 65891788 | 65045 | 13: 66156547 | 65051 | 13: 66242964 |
| 65054 | 13: 66269753 | 65058 | 13: 66340843 | 65068 | 13: 66611398 | 65078 | 13: 66830214 | 65084 | 13: 66995569 |
| 65087 | 13: 67120106 | 65103 | 13: 67448820 | 65126 | 13: 68187572 | 65134 | 13: 68308181 | 65136 | 13: 68378513 |
| 65138 | 13: 68519206 | 65140 | 13: 68528941 | 65142 | 13: 68635086 | 65146 | 13: 68672438 | 65152 | 13: 68880703 |
| 65165 | 13: 69217542 | 65168 | 13: 69272129 | 65170 | 13: 69348741 | 65171 | 13: 69353425 | 65173 | 13: 69380590 |
| 65183 | 13: 69728600 | 65184 | 13: 69731770 | 65187 | 13: 69850661 | 65189 | 13: 69882420 | 65190 | 13: 69890851 |
| 65198 | 13: 70040165 | 65201 | 13: 70082692 | 65207 | 13: 70199117 | 65220 | 13: 70568959 | 65225 | 13: 70651503 |
| 65227 | 13: 70673685 | 65248 | 13: 71190284 | 65267 | 13: 71537155 | 65272 | 13: 71649810 | 65276 | 13: 71778746 |
| 65278 | 13: 71794746 | 65289 | 13: 72099141 | 65290 | 13: 72099160 | 65293 | 13: 72190824 | 65298 | 13: 72223856 |
| 65299 | 13: 72240445 | 65300 | 13: 72247438 | 65308 | 13: 72487250 | 65311 | 13: 72581815 | 65316 | 13: 72835577 |
| 65318 | 13: 72837581 | 65319 | 13: 72837712 | 65320 | 13: 72837736 | 65323 | 13: 72873627 | 65326 | 13: 72911160 |
| 65327 | 13: 72931732 | 65331 | 13: 72989401 | 65333 | 13: 73030191 | 65342 | 13: 73241000 | 65353 | 13: 73412480 |
| 65358 | 13: 73475168 | 65359 | 13: 73486255 | 65360 | 13: 73508637 | 65361 | 13: 73516499 | 65362 | 13: 73552702 |
| 65363 | 13: 73554377 | 65367 | 13: 73602251 | 65369 | 13: 73628006 | 65370 | 13: 73628032 | 65372 | 13: 73657151 |
| 65373 | 13: 73659243 | 65375 | 13: 73684336 | 65376 | 13: 73720189 | 65377 | 13: 73738431 | 65378 | 13: 73747745 |
| 65386 | 13: 74002990 | 65388 | 13: 74075881 | 65396 | 13: 74272611 | 65397 | 13: 74284172 | 65405 | 13: 74425124 |
| 65406 | 13: 74461134 | 65407 | 13: 74464989 | 65408 | 13: 74467020 | 65409 | 13: 74470296 | 65410 | 13: 74476473 |
| 65411 | 13: 74479132 | 65412 | 13: 74479161 | 65417 | 13: 74575589 | 65419 | 13: 74593675 | 65421 | 13: 74635628 |
| 65423 | 13: 74640625 | 65426 | 13: 74665130 | 65427 | 13: 74678375 | 65432 | 13: 74760409 | 65433 | 13: 74815833 |
| 65437 | 13: 74932791 | 65455 | 13: 75521831 | 65463 | 13: 75584984 | 65464 | 13: 75584987 | 65465 | 13: 75585044 |
| 65473 | 13: 75757004 | 65474 | 13: 75767512 | 65478 | 13: 75797537 | 65479 | 13: 75808921 | 65481 | 13: 75817367 |
| 65496 | 13: 76209741 | 65503 | 13: 76376258 | 65506 | 13: 76438473 | 65507 | 13: 76438492 | 65508 | 13: 76442151 |
| 65509 | 13: 76443128 | 65521 | 13: 76672357 | 65523 | 13: 76717525 | 65527 | 13: 76772665 | 65534 | 13: 76884649 |
| 65545 | 13: 77225860 | 65551 | 13: 77281427 | 65555 | 13: 77332710 | 65557 | 13: 77387866 | 65560 | 13: 77423447 |
| 65561 | 13: 77443501 | 65572 | 13: 77715514 | 65574 | 13: 77719500 | 65576 | 13: 77810786 | 65579 | 13: 77851241 |
| 65580 | 13: 77856986 | 65584 | 13: 78019495 | 65589 | 13: 78097466 | 65590 | 13: 78097543 | 65591 | 13: 78097572 |
| 65595 | 13: 78246967 | 65600 | 13: 78323978 | 65601 | 13: 78327563 | 65611 | 13: 78606738 | 65620 | 13: 78732308 |
| 65628 | 13: 78862443 | 65630 | 13: 78895292 | 65636 | 13: 79010034 | 65646 | 13: 79138574 | 65653 | 13: 79289518 |
| 65663 | 13: 79372632 | 65664 | 13: 79378945 | 65670 | 13: 79439113 | 65746 | 13: 80685975 | 65753 | 13: 80868488 |
| 65754 | 13: 80869352 | 65775 | 13: 81361992 | 65780 | 13: 81441602 | 65789 | 13: 81578319 | 65793 | 13: 81659411 |
| 65796 | 13: 81695713 | 65809 | 13: 81975437 | 65811 | 13: 82042702 | 65857 | 13: 82944108 | 65859 | 13: 82944150 |
| 65870 | 13: 83194337 | 65885 | 14: 260388 | 65890 | 14: 344042 | 65892 | 14: 428398 | 65906 | 14: 1024267 |
| 65907 | 14: 1025055 | 65908 | 14: 1033297 | 65909 | 14: 1036134 | 65910 | 14: 1039518 | 65911 | 14: 1044777 |
| 65914 | 14: 1153408 | 65916 | 14: 1188418 | 65919 | 14: 1314992 | 65923 | 14: 1388615 | 65924 | 14: 1394179 |
| 65928 | 14: 1554543 | 65930 | 14: 1658546 | 65931 | 14: 1670892 | 65932 | 14: 1671183 | 65933 | 14: 1671573 |
| 65934 | 14: 1671580 | 65935 | 14: 1681783 | 65936 | 14: 1702340 | 65940 | 14: 1785051 | 65941 | 14: 1798788 |
| 65942 | 14: 1799618 | 65943 | 14: 1799673 | 65944 | 14: 1800049 | 65946 | 14: 1844464 | 65947 | 14: 1854457 |
| 65950 | 14: 1926589 | 65951 | 14: 1946346 | 65966 | 14: 2415920 | 65968 | 14: 2500509 | 65973 | 14: 2669589 |
| 65982 | 14: 2854696 | 65988 | 14: 3015800 | 65989 | 14: 3040833 | 65990 | 14: 3040885 | 65994 | 14: 3097950 |
| 65998 | 14: 3147254 | 66005 | 14: 3206294 | 66006 | 14: 3212884 | 66007 | 14: 3218403 | 66008 | 14: 3218478 |
| 66009 | 14: 3218557 | 66010 | 14: 3221269 | 66011 | 14: 3221338 | 66012 | 14: 3239650 | 66013 | 14: 3239696 |
| 66014 | 14: 3239752 | 66015 | 14: 3239754 | 66016 | 14: 3242336 | 66017 | 14: 3246999 | 66018 | 14: 3247042 |
| 66019 | 14: 3247052 | 66020 | 14: 3278835 | 66021 | 14: 3278838 | 66022 | 14: 3284409 | 66023 | 14: 3284439 |
| 66024 | 14: 3298378 | 66025 | 14: 3305527 | 66026 | 14: 3308936 | 66028 | 14: 3336128 | 66029 | 14: 3336263 |
| 66034 | 14: 3386889 | 66036 | 14: 3433819 | 66038 | 14: 3453301 | 66041 | 14: 3471847 | 66080 | 14: 4216507 |
| 66082 | 14: 4228026 | 66083 | 14: 4228352 | 66085 | 14: 4232919 | 66086 | 14: 4233543 | 66113 | 14: 4912069 |
| 66118 | 14: 5004897 | 66178 | 14: 5998302 | 66185 | 14: 6071169 | 66191 | 14: 6163665 | 66192 | 14: 6174543 |
| 66203 | 14: 6285204 | 66206 | 14: 6295145 | 66207 | 14: 6303201 | 66212 | 14: 6388522 | 66214 | 14: 6472930 |
| 66237 | 14: 6859614 | 66248 | 14: 7064392 | 66250 | 14: 7109542 | 66252 | 14: 7127144 | 66269 | 14: 7497044 |
| 66274 | 14: 7545925 | 66275 | 14: 7545927 | 66276 | 14: 7545936 | 66280 | 14: 7626758 | 66294 | 14: 7895151 |
| 66300 | 14: 7989733 | 66311 | 14: 8058621 | 66312 | 14: 8058649 | 66316 | 14: 8179389 | 66321 | 14: 8301355 |
| 66323 | 14: 8312764 | 66327 | 14: 8394739 | 66328 | 14: 8416607 | 66329 | 14: 8424727 | 66331 | 14: 8441111 |
| 66333 | 14: 8445298 | 66337 | 14: 8540399 | 66338 | 14: 8551277 | 66340 | 14: 8561525 | 66341 | 14: 8566274 |
| 66343 | 14: 8579200 | 66346 | 14: 8601680 | 66347 | 14: 8601681 | 66349 | 14: 8629638 | 66353 | 14: 8664840 |
| 66354 | 14: 8664847 | 66356 | 14: 8672236 | 66359 | 14: 8707161 | 66363 | 14: 8798924 | 66375 | 14: 9035591 |
| 66378 | 14: 9063069 | 66379 | 14: 9064094 | 66380 | 14: 9064111 | 66381 | 14: 9064154 | 66382 | 14: 9064258 |
| 66384 | 14: 9095754 | 66386 | 14: 9121381 | 66387 | 14: 9140334 | 66388 | 14: 9140361 | 66389 | 14: 9140418 |
| 66390 | 14: 9141498 | 66394 | 14: 9188175 | 66400 | 14: 9361706 | 66402 | 14: 9382539 | 66403 | 14: 9399137 |
| 66404 | 14: 9401991 | 66430 | 14: 9976682 | 66431 | 14: 9976704 | 66432 | 14: 9976709 | 66434 | 14: 10038814 |
| 66438 | 14: 10102756 | 66440 | 14: 10152585 | 66443 | 14: 10280749 | 66451 | 14: 10486501 | 66455 | 14: 10530506 |
| 66457 | 14: 10557006 | 66458 | 14: 10566461 | 66459 | 14: 10577632 | 66462 | 14: 10706026 | 66464 | 14: 10756790 |
| 66465 | 14: 10764173 | 66466 | 14: 10788680 | 66467 | 14: 10801527 | 66468 | 14: 10805790 | 66469 | 14: 10807806 |
| 66470 | 14: 10816880 | 66474 | 14: 10893686 | 66476 | 14: 10914761 | 66479 | 14: 10947558 | 66481 | 14: 11009369 |
| 66484 | 14: 11088275 | 66492 | 14: 11252807 | 66504 | 14: 11411289 | 66505 | 14: 11444087 | 66510 | 14: 11540331 |
| 66512 | 14: 11568465 | 66513 | 14: 11575517 | 66516 | 14: 11596900 | 66517 | 14: 11657154 | 66526 | 14: 11763629 |
| 66533 | 14: 11962212 | 66541 | 14: 12136166 | 66546 | 14: 12207418 | 66551 | 14: 12292359 | 66552 | 14: 12293976 |
| 66554 | 14: 12315574 | 66558 | 14: 12384353 | 66559 | 14: 12388961 | 66560 | 14: 12393160 | 66564 | 14: 12514540 |
| 66578 | 14: 12695561 | 66579 | 14: 12710347 | 66593 | 14: 13094541 | 66595 | 14: 13178679 | 66618 | 14: 13920366 |
| 66623 | 14: 14023534 | 66628 | 14: 14069858 | 66631 | 14: 14135529 | 66636 | 14: 14326793 | 66646 | 14: 14560727 |
| 66649 | 14: 14707903 | 66651 | 14: 14728697 | 66652 | 14: 14744411 | 66656 | 14: 14812773 | 66657 | 14: 14823932 |
| 66662 | 14: 14952020 | 66664 | 14: 15013585 | 66666 | 14: 15072256 | 66668 | 14: 15100414 | 66675 | 14: 15318937 |
| 66677 | 14: 15351351 | 66678 | 14: 15369492 | 66682 | 14: 15454870 | 66689 | 14: 15529862 | 66704 | 14: 15827253 |
| 66710 | 14: 15926072 | 66712 | 14: 15954837 | 66715 | 14: 15972218 | 66723 | 14: 16143522 | 66725 | 14: 16177691 |
| 66730 | 14: 16258523 | 66734 | 14: 16293114 | 66737 | 14: 16436394 | 66738 | 14: 16442429 | 66739 | 14: 16450949 |
| 66741 | 14: 16479769 | 66742 | 14: 16493894 | 66752 | 14: 16722564 | 66753 | 14: 16790460 | 66754 | 14: 16817518 |
| 66756 | 14: 16852507 | 66761 | 14: 16906633 | 66762 | 14: 16939530 | 66763 | 14: 16939542 | 66764 | 14: 16939598 |
| 66765 | 14: 16939628 | 66767 | 14: 16969260 | 66771 | 14: 17041215 | 66778 | 14: 17280279 | 66798 | 14: 17770696 |
| 66799 | 14: 17770830 | 66801 | 14: 17799708 | 66802 | 14: 17816110 | 66803 | 14: 17843985 | 66836 | 14: 18601570 |
| 66849 | 14: 18880871 | 66860 | 14: 19181912 | 66865 | 14: 19283893 | 66869 | 14: 19432077 | 66881 | 14: 19699930 |
| 66882 | 14: 19743977 | 66885 | 14: 19813853 | 66890 | 14: 19975802 | 66893 | 14: 19991875 | 66927 | 14: 20847256 |
| 66930 | 14: 20893054 | 66931 | 14: 20896371 | 66932 | 14: 20913913 | 66935 | 14: 20978169 | 66937 | 14: 21048592 |
| 66958 | 14: 21485350 | 66963 | 14: 21604476 | 66974 | 14: 21869418 | 66977 | 14: 21931720 | 66979 | 14: 21998392 |
| 66984 | 14: 22161961 | 66995 | 14: 22407071 | 66996 | 14: 22407076 | 67077 | 14: 22732607 | 67093 | 14: 22882106 |
| 67095 | 14: 22886422 | 67173 | 14: 23215974 | 67345 | 14: 23539160 | 67346 | 14: 23539175 | 67347 | 14: 23539193 |
| 67348 | 14: 23539196 | 67349 | 14: 23539229 | 67424 | 14: 23981200 | 67428 | 14: 24041313 | 67443 | 14: 24389626 |
| 67445 | 14: 24408833 | 67514 | 14: 25111427 | 67519 | 14: 25203874 | 67520 | 14: 25205074 | 67525 | 14: 25258907 |
| 67529 | 14: 25432157 | 67561 | 14: 25864255 | 67565 | 14: 25923336 | 67566 | 14: 25923373 | 67568 | 14: 25985806 |
| 67570 | 14: 25990906 | 67571 | 14: 26012359 | 67573 | 14: 26052398 | 67575 | 14: 26065694 | 67580 | 14: 26140450 |
| 67581 | 14: 26140990 | 67582 | 14: 26141962 | 67583 | 14: 26143779 | 67585 | 14: 26163203 | 67586 | 14: 26170631 |
| 67587 | 14: 26175012 | 67588 | 14: 26176715 | 67590 | 14: 26182412 | 67591 | 14: 26182943 | 67593 | 14: 26184111 |
| 67594 | 14: 26184775 | 67595 | 14: 26185527 | 67596 | 14: 26186351 | 67597 | 14: 26190407 | 67598 | 14: 26191236 |
| 67600 | 14: 26193748 | 67601 | 14: 26194782 | 67602 | 14: 26195880 | 67603 | 14: 26197356 | 67604 | 14: 26209375 |
| 67605 | 14: 26210727 | 67606 | 14: 26212648 | 67607 | 14: 26216203 | 67608 | 14: 26225153 | 67610 | 14: 26246778 |
| 67611 | 14: 26271742 | 67614 | 14: 26308735 | 67617 | 14: 26333506 | 67620 | 14: 26432685 | 67626 | 14: 26633301 |
| 67632 | 14: 26717685 | 67648 | 14: 27181625 | 67649 | 14: 27210120 | 67650 | 14: 27233742 | 67670 | 14: 27952519 |
| 67673 | 14: 28069983 | 67685 | 14: 28479844 | 67691 | 14: 28620485 | 67692 | 14: 28622173 | 67693 | 14: 28624764 |
| 67697 | 14: 28758622 | 67715 | 14: 29296020 | 67725 | 14: 29459640 | 67740 | 14: 29698665 | 67748 | 14: 29894372 |
| 67749 | 14: 29989315 | 67752 | 14: 30012586 | 67767 | 14: 30342088 | 67768 | 14: 30349359 | 67769 | 14: 30356890 |
| 67778 | 14: 30465481 | 67781 | 14: 30499744 | 67794 | 14: 30690571 | 67797 | 14: 30764962 | 67798 | 14: 30811197 |
| 67808 | 14: 31180467 | 67819 | 14: 31555900 | 67820 | 14: 31555992 | 67826 | 14: 31641997 | 67833 | 14: 31874739 |
| 67836 | 14: 31944000 | 67844 | 14: 32203109 | 67866 | 14: 32398203 | 67886 | 14: 32981178 | 67890 | 14: 33060155 |
| 67896 | 14: 33143197 | 67897 | 14: 33143201 | 67900 | 14: 33275732 | 67906 | 14: 33326342 | 67907 | 14: 33342080 |
| 67912 | 14: 33449262 | 67913 | 14: 33458276 | 67919 | 14: 33529983 | 67930 | 14: 33731278 | 67932 | 14: 33839737 |
| 67938 | 14: 33897507 | 67939 | 14: 33921705 | 67942 | 14: 33981693 | 67947 | 14: 34164312 | 67953 | 14: 34301611 |
| 67959 | 14: 34528656 | 67975 | 14: 35058048 | 68018 | 14: 36043204 | 68028 | 14: 36299508 | 68035 | 14: 36373476 |
| 68052 | 14: 36852980 | 68071 | 14: 37488132 | 68084 | 14: 37849675 | 68085 | 14: 37852302 | 68089 | 14: 37925483 |
| 68097 | 14: 38188466 | 68115 | 14: 38814239 | 68116 | 14: 38815880 | 68117 | 14: 38822128 | 68118 | 14: 38822260 |
| 68141 | 14: 39497949 | 68155 | 14: 39876130 | 68156 | 14: 39966401 | 68160 | 14: 40176632 | 68161 | 14: 40176648 |
| 68162 | 14: 40176909 | 68163 | 14: 40176917 | 68164 | 14: 40176996 | 68165 | 14: 40177014 | 68166 | 14: 40177030 |
| 68167 | 14: 40177236 | 68168 | 14: 40177299 | 68179 | 14: 40478861 | 68203 | 14: 41361918 | 68205 | 14: 41438833 |
| 68208 | 14: 41573733 | 68214 | 14: 41689230 | 68217 | 14: 41730144 | 68218 | 14: 41737864 | 68242 | 14: 42286395 |
| 68254 | 14: 42523669 | 68264 | 14: 42805270 | 68273 | 14: 43158811 | 68280 | 14: 43440082 | 68288 | 14: 43769450 |
| 68307 | 14: 44309555 | 68309 | 14: 44430721 | 68318 | 14: 44636417 | 68322 | 14: 44676672 | 68323 | 14: 44676728 |
| 68324 | 14: 44677183 | 68326 | 14: 44690793 | 68327 | 14: 44724666 | 68329 | 14: 44762792 | 68331 | 14: 44776625 |
| 68334 | 14: 44843603 | 68335 | 14: 44854809 | 68336 | 14: 44856580 | 68340 | 14: 44966227 | 68344 | 14: 44988177 |
| 68345 | 14: 45015405 | 68346 | 14: 45018776 | 68347 | 14: 45022201 | 68348 | 14: 45041057 | 68356 | 14: 45253129 |
| 68357 | 14: 45264773 | 68362 | 14: 45445260 | 68378 | 14: 45823929 | 68379 | 14: 45842554 | 68380 | 14: 45845229 |
| 68387 | 14: 45960737 | 68392 | 14: 46043332 | 68395 | 14: 46074189 | 68416 | 14: 46394434 | 68419 | 14: 46425925 |
| 68425 | 14: 46627739 | 68427 | 14: 46701293 | 68437 | 14: 46893352 | 68447 | 14: 47088081 | 68477 | 14: 48079880 |
| 68486 | 14: 48324535 | 68488 | 14: 48324675 | 68491 | 14: 48337571 | 68505 | 14: 48683313 | 68508 | 14: 48746367 |
| 68510 | 14: 48777365 | 68531 | 14: 49324283 | 68534 | 14: 49446616 | 68535 | 14: 49446636 | 68557 | 14: 50162370 |
| 68562 | 14: 50269979 | 68571 | 14: 50641123 | 68586 | 14: 51157346 | 68593 | 14: 51380992 | 68594 | 14: 51381093 |
| 68599 | 14: 51513649 | 68609 | 14: 51705820 | 68618 | 14: 51969375 | 68621 | 14: 52011701 | 68622 | 14: 52087941 |
| 68624 | 14: 52117812 | 68626 | 14: 52174077 | 68627 | 14: 52174084 | 68628 | 14: 52176416 | 68629 | 14: 52176503 |
| 68630 | 14: 52181003 | 68631 | 14: 52193756 | 68632 | 14: 52206425 | 68634 | 14: 52240902 | 68635 | 14: 52240959 |
| 68636 | 14: 52272882 | 68637 | 14: 52274310 | 68647 | 14: 52598698 | 68666 | 14: 53395458 | 68667 | 14: 53433645 |
| 68678 | 14: 53716855 | 68694 | 14: 54155951 | 68699 | 14: 54289749 | 68703 | 14: 54322218 | 68704 | 14: 54323172 |
| 68705 | 14: 54324711 | 68706 | 14: 54326865 | 68707 | 14: 54328568 | 68708 | 14: 54329372 | 68709 | 14: 54330950 |
| 68710 | 14: 54331798 | 68711 | 14: 54332484 | 68712 | 14: 54333187 | 68713 | 14: 54334454 | 68714 | 14: 54335896 |
| 68715 | 14: 54340206 | 68718 | 14: 54366857 | 68719 | 14: 54375801 | 68722 | 14: 54397201 | 68723 | 14: 54441985 |
| 68725 | 14: 54458565 | 68732 | 14: 54625281 | 68745 | 14: 54834484 | 68749 | 14: 55039582 | 68751 | 14: 55074676 |
| 68756 | 14: 55222131 | 68757 | 14: 55226008 | 68758 | 14: 55230280 | 68759 | 14: 55230389 | 68760 | 14: 55283357 |
| 68769 | 14: 55484731 | 68780 | 14: 55685950 | 68789 | 14: 56225330 | 68790 | 14: 56256482 | 68791 | 14: 56276255 |
| 68799 | 14: 56506951 | 68803 | 14: 56545284 | 68804 | 14: 56555253 | 68808 | 14: 56652939 | 68816 | 14: 56827541 |
| 68831 | 14: 57332067 | 68832 | 14: 57346169 | 68833 | 14: 57350713 | 68834 | 14: 57366372 | 68835 | 14: 57383485 |
| 68839 | 14: 57448890 | 68840 | 14: 57459060 | 68842 | 14: 57536798 | 68846 | 14: 57585046 | 68851 | 14: 57728888 |
| 68852 | 14: 57728921 | 68853 | 14: 57779780 | 68854 | 14: 57796975 | 68856 | 14: 57816918 | 68859 | 14: 57922597 |
| 68860 | 14: 57935303 | 68864 | 14: 58056243 | 68868 | 14: 58089515 | 68874 | 14: 58220993 | 68885 | 14: 58495696 |
| 68886 | 14: 58510271 | 68888 | 14: 58533542 | 68892 | 14: 58668510 | 68905 | 14: 58947933 | 68910 | 14: 59026237 |
| 68912 | 14: 59043151 | 68920 | 14: 59231026 | 68947 | 14: 59935092 | 68953 | 14: 60142373 | 68957 | 14: 60181604 |
| 68958 | 14: 60181624 | 68964 | 14: 60282711 | 68965 | 14: 60298957 | 68967 | 14: 60335923 | 68969 | 14: 60400617 |
| 68973 | 14: 60573159 | 68977 | 14: 60646587 | 68978 | 14: 60646600 | 68983 | 14: 60827415 | 68990 | 14: 60986477 |
| 68994 | 14: 61146297 | 68996 | 14: 61230816 | 68997 | 14: 61230819 | 69001 | 14: 61287942 | 69002 | 14: 61313591 |
| 69003 | 14: 61315627 | 69007 | 14: 61411151 | 69008 | 14: 61412641 | 69009 | 14: 61412766 | 69010 | 14: 61412805 |
| 69011 | 14: 61414085 | 69014 | 14: 61513794 | 69028 | 14: 61777801 | 69033 | 14: 61849353 | 69040 | 14: 61948505 |
| 69044 | 14: 62039648 | 69046 | 14: 62081343 | 69049 | 14: 62134019 | 69052 | 14: 62144762 | 69053 | 14: 62144767 |
| 69054 | 14: 62149426 | 69060 | 14: 62299231 | 69068 | 14: 62442888 | 69073 | 14: 62565653 | 69078 | 14: 62687349 |
| 69088 | 14: 62815477 | 69090 | 14: 62881003 | 69100 | 14: 63151822 | 69103 | 14: 63198642 | 69104 | 14: 63225917 |
| 69113 | 14: 63395803 | 69128 | 14: 63850266 | 69131 | 14: 63928813 | 69146 | 14: 64220537 | 69149 | 14: 64336877 |
| 69150 | 14: 64339120 | 69151 | 14: 64353146 | 69156 | 14: 64514888 | 69194 | 14: 65705048 | 69201 | 14: 65784254 |
| 69206 | 14: 65890326 | 69211 | 14: 66085117 | 69212 | 14: 66121609 | 69213 | 14: 66141319 | 69221 | 14: 66266296 |
| 69222 | 14: 66266410 | 69224 | 14: 66350819 | 69225 | 14: 66350850 | 69233 | 14: 66424408 | 69237 | 14: 66604661 |
| 69240 | 14: 66662602 | 69241 | 14: 66669902 | 69242 | 14: 66669945 | 69247 | 14: 66766263 | 69263 | 14: 67192810 |
| 69265 | 14: 67258930 | 69268 | 14: 67326499 | 69270 | 14: 67366402 | 69274 | 14: 67469984 | 69275 | 14: 67472732 |
| 69276 | 14: 67473297 | 69277 | 14: 67473813 | 69278 | 14: 67478743 | 69279 | 14: 67483782 | 69280 | 14: 67495613 |
| 69284 | 14: 67550636 | 69287 | 14: 67603655 | 69290 | 14: 67630731 | 69292 | 14: 67655532 | 69293 | 14: 67660678 |
| 69299 | 14: 67907866 | 69318 | 14: 68256914 | 69323 | 14: 68358639 | 69355 | 14: 69386387 | 69366 | 14: 69774142 |
| 69367 | 14: 69787949 | 69368 | 14: 69802911 | 69371 | 14: 69913935 | 69373 | 14: 69940920 | 69375 | 14: 70002759 |
| 69381 | 14: 70075613 | 69385 | 14: 70170918 | 69386 | 14: 70171506 | 69388 | 14: 70172986 | 69392 | 14: 70218397 |
| 69398 | 14: 70356280 | 69401 | 14: 70440463 | 69403 | 14: 70472924 | 69412 | 14: 70696424 | 69418 | 14: 70903085 |
| 69420 | 14: 70964025 | 69440 | 14: 71594264 | 69447 | 14: 71762783 | 69459 | 14: 72082157 | 69485 | 14: 72739791 |
| 69489 | 14: 72764165 | 69499 | 14: 73108865 | 69504 | 14: 73206090 | 69507 | 14: 73232071 | 69510 | 14: 73246685 |
| 69516 | 14: 73444151 | 69525 | 14: 73731378 | 69529 | 14: 73806578 | 69578 | 14: 75097229 | 69579 | 14: 75097255 |
| 69582 | 14: 75218040 | 69586 | 14: 75379695 | 69587 | 14: 75379698 | 69588 | 14: 75379711 | 69592 | 14: 75591751 |
| 69596 | 14: 75706688 | 69611 | 14: 76100504 | 69612 | 14: 76100657 | 69615 | 14: 76128821 | 69616 | 14: 76129624 |
| 69617 | 14: 76132580 | 69618 | 14: 76138175 | 69619 | 14: 76141064 | 69621 | 14: 76142867 | 69622 | 14: 76144495 |
| 69625 | 14: 76178839 | 69626 | 14: 76178890 | 69628 | 14: 76195852 | 69629 | 14: 76199476 | 69630 | 14: 76207317 |
| 69635 | 14: 76328357 | 69637 | 14: 76366242 | 69646 | 14: 76503558 | 69653 | 14: 76612609 | 69655 | 14: 76770661 |
| 69656 | 14: 76771798 | 69659 | 14: 76845625 | 69661 | 14: 76923277 | 69663 | 14: 76945033 | 69664 | 14: 76964292 |
| 69670 | 14: 77099484 | 69671 | 14: 77174586 | 69672 | 14: 77206279 | 69681 | 14: 77471278 | 69682 | 14: 77471456 |
| 69684 | 14: 77512276 | 69712 | 14: 78249703 | 69713 | 14: 78249764 | 69714 | 14: 78249780 | 69720 | 14: 78305043 |
| 69721 | 14: 78306690 | 69722 | 14: 78312623 | 69725 | 14: 78390822 | 69726 | 14: 78392259 | 69727 | 14: 78393807 |
| 69729 | 14: 78454550 | 69738 | 14: 78651480 | 69747 | 14: 78858840 | 69755 | 14: 79091368 | 69781 | 14: 79790786 |
| 69787 | 14: 79936016 | 69795 | 14: 80077176 | 69796 | 14: 80078158 | 69809 | 14: 80313052 | 69832 | 14: 80855743 |
| 69848 | 14: 81221549 | 69849 | 14: 81264509 | 69850 | 14: 81267100 | 69851 | 14: 81267190 | 69852 | 14: 81267746 |
| 69853 | 14: 81295824 | 69854 | 14: 81295958 | 69861 | 14: 81372244 | 69862 | 14: 81379555 | 69863 | 14: 81392217 |
| 69872 | 14: 81555021 | 69873 | 14: 81583416 | 69876 | 14: 81598835 | 69879 | 14: 81634869 | 69881 | 14: 81683735 |
| 69882 | 14: 81698012 | 69883 | 14: 81701580 | 69887 | 14: 81727910 | 69893 | 14: 81835309 | 69895 | 14: 81857929 |
| 69897 | 14: 81940278 | 69899 | 14: 81972242 | 69903 | 14: 82015406 | 69904 | 14: 82016702 | 69905 | 14: 82026443 |
| 69909 | 14: 82091345 | 69933 | 15: 556424 | 69946 | 15: 1148939 | 69948 | 15: 1192605 | 69953 | 15: 1229858 |
| 69961 | 15: 1387083 | 69962 | 15: 1439384 | 70009 | 15: 2844618 | 70063 | 15: 3928859 | 70079 | 15: 4264863 |
| 70083 | 15: 4294447 | 70092 | 15: 4497670 | 70099 | 15: 4629275 | 70119 | 15: 4985484 | 70122 | 15: 5160844 |
| 70123 | 15: 5160845 | 70124 | 15: 5160859 | 70125 | 15: 5160876 | 70126 | 15: 5160956 | 70128 | 15: 5219937 |
| 70129 | 15: 5234380 | 70130 | 15: 5296961 | 70282 | 15: 5477594 | 70286 | 15: 5550405 | 70289 | 15: 5646426 |
| 70292 | 15: 5686808 | 70293 | 15: 5686891 | 70294 | 15: 5686893 | 70297 | 15: 5779163 | 70300 | 15: 5933216 |
| 70306 | 15: 6041257 | 70320 | 15: 6451524 | 70323 | 15: 6555645 | 70324 | 15: 6579715 | 70329 | 15: 6707584 |
| 70374 | 15: 7579129 | 70391 | 15: 8102324 | 70413 | 15: 8544147 | 70414 | 15: 8547793 | 70425 | 15: 8919907 |
| 70434 | 15: 9119501 | 70444 | 15: 9307296 | 70445 | 15: 9307388 | 70446 | 15: 9308875 | 70449 | 15: 9342376 |
| 70461 | 15: 9653267 | 70462 | 15: 9653311 | 70466 | 15: 9752248 | 70491 | 15: 10716741 | 70493 | 15: 10740715 |
| 70496 | 15: 10827424 | 70505 | 15: 11236303 | 70520 | 15: 11770301 | 70529 | 15: 12167647 | 70540 | 15: 12557589 |
| 70574 | 15: 13886436 | 70586 | 15: 14183988 | 70588 | 15: 14191992 | 70591 | 15: 14203309 | 70594 | 15: 14260966 |
| 70599 | 15: 14448696 | 70600 | 15: 14456572 | 70601 | 15: 14458763 | 70602 | 15: 14459519 | 70603 | 15: 14461707 |
| 70604 | 15: 14462057 | 70605 | 15: 14462078 | 70606 | 15: 14463666 | 70612 | 15: 14705603 | 70617 | 15: 14911197 |
| 70623 | 15: 15068168 | 70624 | 15: 15068209 | 70631 | 15: 15245724 | 70643 | 15: 15417315 | 70644 | 15: 15417341 |
| 70645 | 15: 15417537 | 70646 | 15: 15417584 | 70648 | 15: 15474272 | 70649 | 15: 15505264 | 70651 | 15: 15537455 |
| 70656 | 15: 15691423 | 70657 | 15: 15691442 | 70658 | 15: 15715228 | 70661 | 15: 15789950 | 70664 | 15: 15846394 |
| 70665 | 15: 15857275 | 70666 | 15: 15867538 | 70667 | 15: 15882003 | 70668 | 15: 15887057 | 70671 | 15: 15896498 |
| 70672 | 15: 15896623 | 70673 | 15: 15896626 | 70676 | 15: 15899461 | 70678 | 15: 15941852 | 70685 | 15: 16025726 |
| 70686 | 15: 16034972 | 70688 | 15: 16066600 | 70689 | 15: 16070995 | 70690 | 15: 16086621 | 70691 | 15: 16086825 |
| 70692 | 15: 16090802 | 70693 | 15: 16095590 | 70694 | 15: 16098140 | 70697 | 15: 16106288 | 70698 | 15: 16106299 |
| 70699 | 15: 16106352 | 70700 | 15: 16106429 | 70701 | 15: 16106956 | 70703 | 15: 16181648 | 70720 | 15: 16783559 |
| 70722 | 15: 16876160 | 70725 | 15: 17015197 | 70726 | 15: 17021701 | 70727 | 15: 17023074 | 70728 | 15: 17023080 |
| 70729 | 15: 17035605 | 70730 | 15: 17045413 | 70731 | 15: 17050219 | 70732 | 15: 17051620 | 70734 | 15: 17079996 |
| 70737 | 15: 17110054 | 70739 | 15: 17151448 | 70740 | 15: 17156993 | 70742 | 15: 17174949 | 70743 | 15: 17174997 |
| 70744 | 15: 17178030 | 70748 | 15: 17216169 | 70749 | 15: 17225710 | 70752 | 15: 17272317 | 70771 | 15: 17881167 |
| 70772 | 15: 17905884 | 70773 | 15: 17907768 | 70774 | 15: 17907803 | 70776 | 15: 17984528 | 70780 | 15: 18132345 |
| 70788 | 15: 18340406 | 70789 | 15: 18340444 | 70790 | 15: 18340452 | 70791 | 15: 18340495 | 70792 | 15: 18340522 |
| 70800 | 15: 18582994 | 70828 | 15: 19344195 | 70852 | 15: 20040778 | 70853 | 15: 20041510 | 70855 | 15: 20059908 |
| 70861 | 15: 20159086 | 70862 | 15: 20208131 | 70863 | 15: 20239028 | 70864 | 15: 20251395 | 70874 | 15: 20506266 |
| 70898 | 15: 20943851 | 70900 | 15: 20983899 | 70904 | 15: 21079829 | 70909 | 15: 21144577 | 70910 | 15: 21222779 |
| 70915 | 15: 21328250 | 70916 | 15: 21331201 | 70917 | 15: 21344247 | 70927 | 15: 21713133 | 70928 | 15: 21715125 |
| 70930 | 15: 21724458 | 70937 | 15: 21944261 | 70938 | 15: 21955334 | 70939 | 15: 21959233 | 70940 | 15: 21980185 |
| 70944 | 15: 22109125 | 70947 | 15: 22219010 | 70950 | 15: 22224493 | 70951 | 15: 22255512 | 70959 | 15: 22397563 |
| 70988 | 15: 23032442 | 70992 | 15: 23140676 | 70995 | 15: 23230388 | 71002 | 15: 23361381 | 71003 | 15: 23382217 |
| 71006 | 15: 23428462 | 71007 | 15: 23428524 | 71008 | 15: 23428581 | 71009 | 15: 23495909 | 71023 | 15: 23729866 |
| 71024 | 15: 23732149 | 71059 | 15: 23865919 | 71072 | 15: 24238065 | 71074 | 15: 24278519 | 71083 | 15: 24394620 |
| 71084 | 15: 24406959 | 71103 | 15: 24778258 | 71104 | 15: 24778597 | 71105 | 15: 24783092 | 71145 | 15: 25774695 |
| 71150 | 15: 25820329 | 71157 | 15: 25920672 | 71166 | 15: 26130885 | 71170 | 15: 26196504 | 71175 | 15: 26276504 |
| 71176 | 15: 26297137 | 71192 | 15: 26653665 | 71199 | 15: 26809489 | 71211 | 15: 27151330 | 71219 | 15: 27430624 |
| 71223 | 15: 27474421 | 71226 | 15: 27503920 | 71228 | 15: 27522579 | 71230 | 15: 27581168 | 71236 | 15: 27795197 |
| 71240 | 15: 27909371 | 71243 | 15: 27975706 | 71246 | 15: 28011102 | 71247 | 15: 28029857 | 71248 | 15: 28029959 |
| 71249 | 15: 28047023 | 71251 | 15: 28062942 | 71256 | 15: 28137955 | 71257 | 15: 28138113 | 71260 | 15: 28217846 |
| 71261 | 15: 28233069 | 71264 | 15: 28261207 | 71276 | 15: 28502744 | 71285 | 15: 28668348 | 71286 | 15: 28669780 |
| 71287 | 15: 28674486 | 71288 | 15: 28675386 | 71289 | 15: 28678307 | 71290 | 15: 28679179 | 71292 | 15: 28683279 |
| 71293 | 15: 28687052 | 71294 | 15: 28689278 | 71304 | 15: 28889609 | 71305 | 15: 28903033 | 71306 | 15: 28903036 |
| 71307 | 15: 28904856 | 71311 | 15: 28935265 | 71314 | 15: 28955129 | 71315 | 15: 28957203 | 71316 | 15: 28995849 |
| 71318 | 15: 29029067 | 71320 | 15: 29051391 | 71327 | 15: 29217795 | 71331 | 15: 29377994 | 71338 | 15: 29599939 |
| 71341 | 15: 29669861 | 71342 | 15: 29691482 | 71343 | 15: 29695545 | 71344 | 15: 29708525 | 71358 | 15: 30156144 |
| 71359 | 15: 30182678 | 71372 | 15: 30495429 | 71376 | 15: 30550572 | 71378 | 15: 30571590 | 71379 | 15: 30592484 |
| 71381 | 15: 30681136 | 71387 | 15: 30887853 | 71395 | 15: 31076884 | 71398 | 15: 31119240 | 71423 | 15: 31752349 |
| 71426 | 15: 31762344 | 71427 | 15: 31762695 | 71428 | 15: 31762716 | 71429 | 15: 31762815 | 71430 | 15: 31766858 |
| 71431 | 15: 31772217 | 71432 | 15: 31772367 | 71433 | 15: 31772508 | 71435 | 15: 31789881 | 71439 | 15: 31967339 |
| 71447 | 15: 32184560 | 71448 | 15: 32184561 | 71455 | 15: 32326526 | 71457 | 15: 32408675 | 71458 | 15: 32415848 |
| 71473 | 15: 32775518 | 71475 | 15: 32857841 | 71479 | 15: 32885957 | 71481 | 15: 32912616 | 71482 | 15: 32919523 |
| 71487 | 15: 33030880 | 71493 | 15: 33140018 | 71495 | 15: 33140162 | 71496 | 15: 33143682 | 71499 | 15: 33200022 |
| 71502 | 15: 33313146 | 71505 | 15: 33357725 | 71513 | 15: 33683094 | 71522 | 15: 33855059 | 71546 | 15: 34227669 |
| 71554 | 15: 34378606 | 71558 | 15: 34416916 | 71563 | 15: 34514270 | 71564 | 15: 34527315 | 71572 | 15: 34677976 |
| 71574 | 15: 34706081 | 71576 | 15: 34775584 | 71578 | 15: 34801602 | 71582 | 15: 34866384 | 71583 | 15: 34892433 |
| 71588 | 15: 34974291 | 71591 | 15: 35040731 | 71592 | 15: 35049691 | 71598 | 15: 35277152 | 71599 | 15: 35337946 |
| 71604 | 15: 35461378 | 71611 | 15: 35646977 | 71612 | 15: 35648952 | 71615 | 15: 35707211 | 71617 | 15: 35715226 |
| 71635 | 15: 36132660 | 71636 | 15: 36169165 | 71639 | 15: 36183126 | 71640 | 15: 36219316 | 71645 | 15: 36321976 |
| 71655 | 15: 36576380 | 71657 | 15: 36603077 | 71678 | 15: 37088135 | 71684 | 15: 37186172 | 71706 | 15: 37547916 |
| 71731 | 15: 38265442 | 71738 | 15: 38472609 | 71769 | 15: 39119041 | 71776 | 15: 39333095 | 71777 | 15: 39347738 |
| 71781 | 15: 39412550 | 71785 | 15: 39545268 | 71799 | 15: 39871286 | 71804 | 15: 39927546 | 71805 | 15: 39927550 |
| 71806 | 15: 39927601 | 71809 | 15: 40067276 | 71812 | 15: 40162512 | 71817 | 15: 40279882 | 71818 | 15: 40279893 |
| 71819 | 15: 40279894 | 71820 | 15: 40280012 | 71823 | 15: 40449461 | 71824 | 15: 40449484 | 71826 | 15: 40502987 |
| 71832 | 15: 40632071 | 71833 | 15: 40641136 | 71835 | 15: 40723287 | 71837 | 15: 40759558 | 71838 | 15: 40847130 |
| 71845 | 15: 40955841 | 71849 | 15: 41014898 | 71850 | 15: 41016306 | 71852 | 15: 41150753 | 71860 | 15: 41290689 |
| 71869 | 15: 41440324 | 71877 | 15: 41698377 | 71878 | 15: 41698492 | 71879 | 15: 41698511 | 71880 | 15: 41705974 |
| 71881 | 15: 41705975 | 71882 | 15: 41706097 | 71885 | 15: 41765753 | 71886 | 15: 41767083 | 71887 | 15: 41768163 |
| 71889 | 15: 41814640 | 71901 | 15: 42032808 | 71904 | 15: 42124927 | 71907 | 15: 42185672 | 71909 | 15: 42231274 |
| 71912 | 15: 42264995 | 71924 | 15: 42509261 | 71926 | 15: 42590389 | 71932 | 15: 42752352 | 71934 | 15: 42787098 |
| 71936 | 15: 42818617 | 71939 | 15: 42848256 | 71940 | 15: 42854523 | 71951 | 15: 43042213 | 71952 | 15: 43045041 |
| 71953 | 15: 43046535 | 71954 | 15: 43049707 | 71955 | 15: 43053427 | 71956 | 15: 43055412 | 71957 | 15: 43056476 |
| 71958 | 15: 43057569 | 71959 | 15: 43058822 | 71960 | 15: 43059592 | 71962 | 15: 43063078 | 71963 | 15: 43063710 |
| 71964 | 15: 43097237 | 71965 | 15: 43098155 | 71970 | 15: 43259917 | 71971 | 15: 43260963 | 71972 | 15: 43262025 |
| 71973 | 15: 43262890 | 71974 | 15: 43269560 | 71975 | 15: 43276395 | 71976 | 15: 43286382 | 71977 | 15: 43288762 |
| 71978 | 15: 43292368 | 71979 | 15: 43293082 | 71980 | 15: 43294086 | 71989 | 15: 43476234 | 71993 | 15: 43560696 |
| 71994 | 15: 43561701 | 71995 | 15: 43563456 | 71996 | 15: 43566385 | 71997 | 15: 43573222 | 71998 | 15: 43580011 |
| 72000 | 15: 43625295 | 72002 | 15: 43642580 | 72003 | 15: 43664227 | 72007 | 15: 43741512 | 72020 | 15: 43936008 |
| 72022 | 15: 43939348 | 72023 | 15: 43939353 | 72024 | 15: 43939488 | 72046 | 15: 44460580 | 72047 | 15: 44479177 |
| 72056 | 15: 44643217 | 72058 | 15: 44735861 | 72075 | 15: 45129015 | 72083 | 15: 45210490 | 72084 | 15: 45221891 |
| 72085 | 15: 45246954 | 72088 | 15: 45346073 | 72090 | 15: 45379221 | 72091 | 15: 45415112 | 72103 | 15: 45716064 |
| 72104 | 15: 45722551 | 72109 | 15: 45827220 | 72126 | 15: 46426137 | 72128 | 15: 46464105 | 72131 | 15: 46490284 |
| 72133 | 15: 46492960 | 72134 | 15: 46494185 | 72135 | 15: 46500386 | 72136 | 15: 46500459 | 72137 | 15: 46500629 |
| 72138 | 15: 46500752 | 72139 | 15: 46505669 | 72140 | 15: 46506092 | 72141 | 15: 46506887 | 72142 | 15: 46506889 |
| 72143 | 15: 46506939 | 72144 | 15: 46507314 | 72145 | 15: 46507929 | 72146 | 15: 46508001 | 72147 | 15: 46508021 |
| 72148 | 15: 46512024 | 72149 | 15: 46512113 | 72156 | 15: 46613300 | 72159 | 15: 46636679 | 72161 | 15: 46645269 |
| 72162 | 15: 46646092 | 72173 | 15: 46864410 | 72174 | 15: 46866871 | 72178 | 15: 46967802 | 72189 | 15: 47187912 |
| 72190 | 15: 47188138 | 72191 | 15: 47188507 | 72201 | 15: 47615122 | 72202 | 15: 47641029 | 72205 | 15: 47779635 |
| 72206 | 15: 47779640 | 72207 | 15: 47779664 | 72208 | 15: 47779674 | 72209 | 15: 47787011 | 72210 | 15: 47787118 |
| 72211 | 15: 47848072 | 72212 | 15: 47856278 | 72213 | 15: 47857368 | 72215 | 15: 47862235 | 72216 | 15: 47862345 |
| 72217 | 15: 47867091 | 72222 | 15: 48008735 | 72224 | 15: 48040175 | 72225 | 15: 48040515 | 72226 | 15: 48040954 |
| 72228 | 15: 48078030 | 72229 | 15: 48078755 | 72230 | 15: 48102626 | 72231 | 15: 48102703 | 72232 | 15: 48103271 |
| 72233 | 15: 48103402 | 72250 | 15: 48373937 | 72251 | 15: 48378524 | 72252 | 15: 48386761 | 72253 | 15: 48390883 |
| 72254 | 15: 48396110 | 72255 | 15: 48397484 | 72266 | 15: 48867306 | 72267 | 15: 48952912 | 72275 | 15: 49295120 |
| 72287 | 15: 49686635 | 72289 | 15: 49798310 | 72292 | 15: 49812025 | 72296 | 15: 50073269 | 72302 | 15: 50348347 |
| 72305 | 15: 50405124 | 72306 | 15: 50418732 | 72311 | 15: 50513682 | 72312 | 15: 50513924 | 72326 | 15: 51057213 |
| 72329 | 15: 51193044 | 72332 | 15: 51271630 | 72333 | 15: 51275342 | 72336 | 15: 51360447 | 72337 | 15: 51360592 |
| 72338 | 15: 51362026 | 72339 | 15: 51362043 | 72340 | 15: 51362061 | 72341 | 15: 51362080 | 72342 | 15: 51362276 |
| 72343 | 15: 51362277 | 72344 | 15: 51362431 | 72345 | 15: 51362457 | 72346 | 15: 51362458 | 72347 | 15: 51364116 |
| 72348 | 15: 51364137 | 72349 | 15: 51420115 | 72350 | 15: 51441064 | 72351 | 15: 51441095 | 72352 | 15: 51511445 |
| 72354 | 15: 51518000 | 72355 | 15: 51519061 | 72357 | 15: 51521753 | 72358 | 15: 51523084 | 72359 | 15: 51529172 |
| 72360 | 15: 51530078 | 72361 | 15: 51535001 | 72362 | 15: 51542928 | 72366 | 15: 51558105 | 72367 | 15: 51563195 |
| 72370 | 15: 51568569 | 72371 | 15: 51576564 | 72372 | 15: 51610810 | 72376 | 15: 51684740 | 72377 | 15: 51687709 |
| 72379 | 15: 51689738 | 72382 | 15: 51736976 | 72384 | 15: 51790467 | 72386 | 15: 51803577 | 72394 | 15: 51988641 |
| 72397 | 15: 52007899 | 72404 | 15: 52185081 | 72406 | 15: 52200879 | 72413 | 15: 52360124 | 72424 | 15: 52658762 |
| 72425 | 15: 52660106 | 72426 | 15: 52673691 | 72441 | 15: 53067270 | 72445 | 15: 53137213 | 72449 | 15: 53308023 |
| 72451 | 15: 53309880 | 72452 | 15: 53310424 | 72453 | 15: 53311034 | 72454 | 15: 53311897 | 72455 | 15: 53312462 |
| 72456 | 15: 53314094 | 72457 | 15: 53314682 | 72458 | 15: 53316670 | 72459 | 15: 53319540 | 72460 | 15: 53320533 |
| 72461 | 15: 53321307 | 72462 | 15: 53323633 | 72463 | 15: 53324367 | 72465 | 15: 53325750 | 72466 | 15: 53326273 |
| 72467 | 15: 53326849 | 72468 | 15: 53327898 | 72469 | 15: 53332122 | 72471 | 15: 53333616 | 72472 | 15: 53338788 |
| 72476 | 15: 53398368 | 72478 | 15: 53441398 | 72486 | 15: 53709788 | 7248 | 15: 53727634 | 72488 | 15: 53729356 |
| 72489 | 15: 53738936 | 72490 | 15: 53743609 | 72491 | 15: 53753517 | 72492 | 15: 53761189 | 72494 | 15: 53788777 |
| 72495 | 15: 53796595 | 72496 | 15: 53800970 | 72502 | 15: 53945334 | 72518 | 15: 54255643 | 72527 | 15: 54462755 |
| 72530 | 15: 54489427 | 72531 | 15: 54500400 | 72541 | 15: 54748501 | 72544 | 15: 54834057 | 72548 | 15: 54885008 |
| 72549 | 15: 54885114 | 72550 | 15: 54885117 | 72551 | 15: 54889719 | 72552 | 15: 54889820 | 72556 | 15: 55119302 |
| 72565 | 15: 55289790 | 72569 | 15: 55444251 | 72574 | 15: 55529280 | 72582 | 15: 55734507 | 72589 | 15: 56022102 |
| 72596 | 15: 56200579 | 72603 | 15: 56497282 | 72604 | 15: 56497450 | 72605 | 15: 56501007 | 72607 | 15: 56515635 |
| 72608 | 15: 56515639 | 72609 | 15: 56515642 | 72610 | 15: 56515714 | 72611 | 15: 56518845 | 72612 | 15: 56518860 |
| 72613 | 15: 56518880 | 72614 | 15: 56518892 | 72615 | 15: 56518948 | 72616 | 15: 56519916 | 72617 | 15: 56520003 |
| 72618 | 15: 56520009 | 72619 | 15: 56522354 | 72620 | 15: 56523085 | 72621 | 15: 56523145 | 72622 | 15: 56525703 |
| 72623 | 15: 56526321 | 72624 | 15: 56527735 | 72625 | 15: 56529668 | 72626 | 15: 56548649 | 72644 | 15: 56809212 |
| 72675 | 15: 57003970 | 72684 | 15: 57272924 | 72689 | 15: 57423300 | 72692 | 15: 57500020 | 72694 | 15: 57536234 |
| 72695 | 15: 57536250 | 72703 | 15: 57718142 | 72709 | 15: 57803493 | 72712 | 15: 57854603 | 72722 | 15: 58001428 |
| 72727 | 15: 58088362 | 72734 | 15: 58241375 | 72736 | 15: 58282071 | 72737 | 15: 58283033 | 72740 | 15: 58330257 |
| 72760 | 15: 58671427 | 72773 | 15: 59112131 | 72787 | 15: 59537850 | 72795 | 15: 59796390 | 72809 | 15: 60285268 |
| 72813 | 15: 60368718 | 72815 | 15: 60428854 | 72820 | 15: 60523980 | 72823 | 15: 60608578 | 72850 | 15: 61257843 |
| 72854 | 15: 61342496 | 72868 | 15: 61603248 | 72869 | 15: 61639615 | 72871 | 15: 61721710 | 72872 | 15: 61721767 |
| 72873 | 15: 61724284 | 72874 | 15: 61724369 | 72875 | 15: 61724392 | 72876 | 15: 61725702 | 72879 | 15: 61767375 |
| 72894 | 15: 62032399 | 72907 | 15: 62467385 | 72912 | 15: 62561316 | 72914 | 15: 62563939 | 72915 | 15: 62586048 |
| 72916 | 15: 62586078 | 72920 | 15: 62652293 | 72938 | 15: 63066650 | 72940 | 15: 63100087 | 72941 | 15: 63107739 |
| 72949 | 15: 63295827 | 72951 | 15: 63300815 | 72952 | 15: 63303147 | 72960 | 15: 63529465 | 72962 | 15: 63574887 |
| 72964 | 15: 63579300 | 72969 | 15: 63631630 | 72972 | 15: 63722937 | 72973 | 15: 63723070 | 72974 | 15: 63743524 |
| 72979 | 15: 63917098 | 72983 | 15: 63967865 | 72997 | 15: 64256628 | 72999 | 15: 64283258 | 73001 | 15: 64333153 |
| 73004 | 15: 64416446 | 73023 | 15: 64714146 | 73026 | 15: 64744225 | 73027 | 15: 64771598 | 73031 | 15: 64801402 |
| 73033 | 15: 64806923 | 73038 | 15: 64854504 | 73040 | 15: 64908292 | 73046 | 15: 64986216 | 73050 | 15: 65029608 |
| 73056 | 15: 65148971 | 73064 | 15: 65275286 | 73066 | 15: 65335638 | 73075 | 15: 65436899 | 73078 | 15: 65521232 |
| 73080 | 15: 65594811 | 73081 | 15: 65607689 | 73083 | 15: 65622127 | 73085 | 15: 65656084 | 73086 | 15: 65670390 |
| 73091 | 15: 65822176 | 73093 | 15: 65854403 | 73105 | 15: 66145675 | 73106 | 15: 66156197 | 73115 | 15: 66337078 |
| 73117 | 15: 66339107 | 73119 | 15: 66380166 | 73121 | 15: 66418487 | 73124 | 15: 66460251 | 73127 | 15: 66496173 |
| 73129 | 15: 66543651 | 73141 | 15: 66724079 | 73146 | 15: 66881298 | 73154 | 15: 67179102 | 73164 | 15: 67470477 |
| 73176 | 15: 67880702 | 73196 | 15: 68313970 | 73199 | 15: 68378183 | 73216 | 15: 68982890 | 73217 | 15: 69062551 |
| 73224 | 15: 69173709 | 73249 | 15: 70010700 | 73255 | 15: 70308624 | 73259 | 15: 70429364 | 73262 | 15: 70465643 |
| 73282 | 15: 71042092 | 73289 | 15: 71280393 | 73299 | 15: 71468201 | 73300 | 15: 71468245 | 73315 | 15: 71909279 |
| 73349 | 15: 72924654 | 73350 | 15: 72992522 | 73355 | 15: 73041310 | 73358 | 15: 73097589 | 73360 | 15: 73124963 |
| 73362 | 15: 73143263 | 73363 | 15: 73144008 | 73364 | 15: 73144009 | 73365 | 15: 73144095 | 73366 | 15: 73144112 |
| 73367 | 15: 73144117 | 73368 | 15: 73144142 | 73372 | 15: 73231049 | 73373 | 15: 73250550 | 73376 | 15: 73270606 |
| 73385 | 15: 73422749 | 73386 | 15: 73470922 | 73389 | 15: 73555524 | 73390 | 15: 73555544 | 73391 | 15: 73555546 |
| 73392 | 15: 73555549 | 73394 | 15: 73590486 | 73400 | 15: 73747012 | 73407 | 15: 74016858 | 73408 | 15: 74019876 |
| 73409 | 15: 74047524 | 73412 | 15: 74101712 | 73413 | 15: 74120008 | 73421 | 15: 74204147 | 73422 | 15: 74221807 |
| 73423 | 15: 74236733 | 73440 | 15: 74680300 | 73442 | 15: 74706685 | 73443 | 15: 74715602 | 73444 | 15: 74730746 |
| 73445 | 15: 74736252 | 73448 | 15: 74763309 | 73449 | 15: 74765962 | 73456 | 15: 74857238 | 73476 | 15: 75401306 |
| 73484 | 15: 75520621 | 73496 | 15: 75764247 | 73498 | 15: 75807751 | 73505 | 15: 76018207 | 73513 | 15: 76268285 |
| 73514 | 15: 76284024 | 73515 | 15: 76295386 | 73517 | 15: 76357310 | 73519 | 15: 76369909 | 73520 | 15: 76423356 |
| 73525 | 15: 76490114 | 73530 | 15: 76627029 | 73531 | 15: 76628527 | 73533 | 15: 76676572 | 73542 | 15: 76909234 |
| 73543 | 15: 76913709 | 73554 | 15: 77213130 | 73558 | 15: 77256270 | 73561 | 15: 77333647 | 73563 | 15: 77341921 |
| 73569 | 15: 77457892 | 73570 | 15: 77533291 | 73572 | 15: 77565837 | 73573 | 15: 77581726 | 73574 | 15: 77581747 |
| 73575 | 15: 77581752 | 73576 | 15: 77581872 | 73577 | 15: 77581882 | 73580 | 15: 77638946 | 73582 | 15: 77676086 |
| 73590 | 15: 77841257 | 73591 | 15: 77844974 | 73597 | 15: 77988330 | 73598 | 15: 77989577 | 73600 | 15: 77997353 |
| 73601 | 15: 78001161 | 73603 | 15: 78001840 | 73604 | 15: 78003499 | 73605 | 15: 78004209 | 73606 | 15: 78008228 |
| 73607 | 15: 78010723 | 73608 | 15: 78011919 | 73610 | 15: 78017138 | 73613 | 15: 78027580 | 73614 | 15: 78028904 |
| 73615 | 15: 78032066 | 73616 | 15: 78033166 | 73617 | 15: 78033926 | 73618 | 15: 78036014 | 73619 | 15: 78037096 |
| 73620 | 15: 78043902 | 73621 | 15: 78049025 | 73623 | 15: 78065440 | 73624 | 15: 78069489 | 73637 | 15: 78497070 |
| 73643 | 15: 78705678 | 73644 | 15: 78711863 | 73654 | 15: 79013310 | 73662 | 15: 79316918 | 73663 | 15: 79316919 |
| 73665 | 15: 79446531 | 73666 | 15: 79447076 | 73667 | 15: 79447126 | 73668 | 15: 79447145 | 73671 | 15: 79651229 |
| 73672 | 15: 79651253 | 73684 | 15: 79994534 | 73699 | 15: 80313710 | 73702 | 15: 80329440 | 73707 | 15: 80387982 |
| 73711 | 15: 80447483 | 73714 | 15: 80528280 | 73722 | 15: 80779796 | 73724 | 15: 80825322 | 73725 | 15: 80846525 |
| 73728 | 15: 80988789 | 73733 | 15: 81116878 | 73735 | 15: 81144733 | 73736 | 15: 81149287 | 73737 | 15: 81161372 |
| 73739 | 15: 81168190 | 73744 | 15: 81197998 | 73751 | 15: 81267470 | 73752 | 15: 81279270 | 73759 | 15: 81372132 |
| 73775 | 15: 81857983 | 73793 | 15: 82380481 | 73799 | 15: 82629156 | 73806 | 15: 82720061 | 73807 | 15: 82720089 |
| 73810 | 15: 82745967 | 73811 | 15: 82746237 | 73815 | 15: 82788081 | 73817 | 15: 82797551 | 73819 | 15: 82843451 |
| 73820 | 15: 82851394 | 73824 | 15: 82933219 | 73825 | 15: 82949122 | 73830 | 15: 83006747 | 73831 | 15: 83013517 |
| 73832 | 15: 83013534 | 73840 | 15: 83073328 | 73848 | 15: 83277486 | 73849 | 15: 83288242 | 73850 | 15: 83309084 |
| 73855 | 15: 83458586 | 73865 | 15: 83567476 | 73866 | 15: 83583523 | 73867 | 15: 83586864 | 73868 | 15: 83586930 |
| 73911 | 16: 752827 | 73914 | 16: 766023 | 73915 | 16: 804997 | 73916 | 16: 806537 | 73922 | 16: 921175 |
| 73929 | 16: 978613 | 73937 | 16: 1181761 | 73939 | 16: 1204788 | 73941 | 16: 1214929 | 73946 | 16: 1324936 |
| 73963 | 16: 1582068 | 73964 | 16: 1599816 | 73965 | 16: 1625990 | 73988 | 16: 2055227 | 73992 | 16: 2194637 |
| 73993 | 16: 2224824 | 73995 | 16: 2248476 | 73996 | 16: 2263389 | 73997 | 16: 2277642 | 74004 | 16: 2402507 |
| 74005 | 16: 2467947 | 74013 | 16: 2602159 | 74017 | 16: 2743842 | 74023 | 16: 2938620 | 74026 | 16: 3018942 |
| 74030 | 16: 3067157 | 74038 | 16: 3201109 | 74040 | 16: 3201263 | 74042 | 16: 3253128 | 74043 | 16: 3269182 |
| 74044 | 16: 3269280 | 74045 | 16: 3270297 | 74051 | 16: 3378716 | 74055 | 16: 3405375 | 74059 | 16: 3465967 |
| 74062 | 16: 3607615 | 74065 | 16: 3643356 | 74066 | 16: 3644483 | 74073 | 16: 3791803 | 74080 | 16: 3992289 |
| 74087 | 16: 4106339 | 74088 | 16: 4109307 | 74093 | 16: 4171303 | 74099 | 16: 4262626 | 74100 | 16: 4262650 |
| 74101 | 16: 4265692 | 74103 | 16: 4290878 | 74109 | 16: 4423692 | 74114 | 16: 4475911 | 74119 | 16: 4592258 |
| 74122 | 16: 4640118 | 74125 | 16: 4717716 | 74129 | 16: 4872430 | 74130 | 16: 4898040 | 74133 | 16: 4999290 |
| 74140 | 16: 5116140 | 74146 | 16: 5243133 | 74147 | 16: 5265259 | 74148 | 16: 5265363 | 74162 | 16: 6052639 |
| 74164 | 16: 6060113 | 74166 | 16: 6207254 | 74168 | 16: 6308221 | 74169 | 16: 6317267 | 74170 | 16: 6318112 |
| 74171 | 16: 6319483 | 74172 | 16: 6323633 | 74173 | 16: 6325179 | 74175 | 16: 6332032 | 74176 | 16: 6333222 |
| 74177 | 16: 6335935 | 74180 | 16: 6376238 | 74191 | 16: 6749567 | 74204 | 16: 7268343 | 74214 | 16: 7503961 |
| 74270 | 16: 9734287 | 74280 | 16: 9996877 | 74307 | 16: 10894806 | 74318 | 16: 11281651 | 74336 | 16: 11756836 |
| 74343 | 16: 11874391 | 74347 | 16: 11985702 | 74364 | 16: 12483562 | 74365 | 16: 12486013 | 74378 | 16: 12756820 |
| 74383 | 16: 12870981 | 74400 | 16: 13072437 | 74411 | 16: 13341826 | 74415 | 16: 13479712 | 74431 | 16: 13921125 |
| 74441 | 16: 14274292 | 74444 | 16: 14375476 | 74450 | 16: 14512750 | 74456 | 16: 14612740 | 74457 | 16: 14612809 |
| 74485 | 16: 15352405 | 74486 | 16: 15355419 | 74514 | 16: 16201608 | 74519 | 16: 16433665 | 74520 | 16: 16433701 |
| 74532 | 16: 16902875 | 74586 | 16: 18664935 | 74596 | 16: 18957732 | 74624 | 16: 19622167 | 74629 | 16: 19786076 |
| 74645 | 16: 20175121 | 74649 | 16: 20294926 | 74659 | 16: 20540783 | 74660 | 16: 20555295 | 74674 | 16: 20905213 |
| 74676 | 16: 20943232 | 74715 | 16: 21954699 | 74722 | 16: 22188120 | 74724 | 16: 22316546 | 74726 | 16: 22390334 |
| 74729 | 16: 22420005 | 74742 | 16: 22743613 | 74744 | 16: 22775537 | 74745 | 16: 22786666 | 74762 | 16: 23286838 |
| 74768 | 16: 23427214 | 74769 | 16: 23439282 | 74770 | 16: 23508581 | 74771 | 16: 23518024 | 74772 | 16: 23544281 |
| 74773 | 16: 23557997 | 74776 | 16: 23621715 | 74782 | 16: 23717545 | 74783 | 16: 23721462 | 74792 | 16: 23915566 |
| 74793 | 16: 23918537 | 74801 | 16: 24174300 | 74802 | 16: 24198154 | 74806 | 16: 24253488 | 74809 | 16: 24321784 |
| 74822 | 16: 24770805 | 74844 | 16: 24996364 | 74847 | 16: 25007172 | 74856 | 16: 25057711 | 74879 | 16: 25209115 |
| 74900 | 16: 25434176 | 74908 | 16: 25723685 | 74909 | 16: 25753006 | 74910 | 16: 25760167 | 74911 | 16: 25845363 |
| 74915 | 16: 25999582 | 74916 | 16: 26009236 | 74917 | 16: 26012436 | 74918 | 16: 26021774 | 74920 | 16: 26036756 |
| 74921 | 16: 26037402 | 74924 | 16: 26139905 | 74925 | 16: 26150072 | 74926 | 16: 26150074 | 74927 | 16: 26153413 |
| 74929 | 16: 26186921 | 74930 | 16: 26216113 | 74931 | 16: 26217301 | 74933 | 16: 26220845 | 74934 | 16: 26227564 |
| 74935 | 16: 26274073 | 74936 | 16: 26296242 | 74940 | 16: 26352124 | 74941 | 16: 26356312 | 74943 | 16: 26451726 |
| 74951 | 16: 26637237 | 74973 | 16: 27186026 | 74989 | 16: 27756698 | 74996 | 16: 27880735 | 74997 | 16: 27880880 |
| 75002 | 16: 27952368 | 75003 | 16: 28008613 | 75004 | 16: 28008626 | 75013 | 16: 28274084 | 75024 | 16: 28665499 |
| 75034 | 16: 28945983 | 75036 | 16: 29003100 | 75057 | 16: 29692650 | 75059 | 16: 29709436 | 75060 | 16: 29760720 |
| 75061 | 16: 29760768 | 75064 | 16: 29872847 | 75065 | 16: 29893692 | 75079 | 16: 30287006 | 75081 | 16: 30348202 |
| 75088 | 16: 30604134 | 75092 | 16: 30729515 | 75096 | 16: 30772837 | 75098 | 16: 30794250 | 75099 | 16: 30795047 |
| 75100 | 16: 30802513 | 75102 | 16: 30839358 | 75108 | 16: 31046070 | 75109 | 16: 31054739 | 75120 | 16: 31360463 |
| 75130 | 16: 31587509 | 75131 | 16: 31613864 | 75137 | 16: 31734054 | 75146 | 16: 31961054 | 75147 | 16: 31961057 |
| 75151 | 16: 32067908 | 75157 | 16: 32356916 | 75162 | 16: 32428255 | 75165 | 16: 32550214 | 75180 | 16: 32851343 |
| 75183 | 16: 32875785 | 75184 | 16: 32880400 | 75187 | 16: 32951618 | 75188 | 16: 32951739 | 75215 | 16: 33649281 |
| 75216 | 16: 33650436 | 75220 | 16: 33885770 | 75224 | 16: 33965943 | 75226 | 16: 33984101 | 75247 | 16: 34572625 |
| 75250 | 16: 34620173 | 75254 | 16: 34722052 | 75257 | 16: 34852541 | 75262 | 16: 34933617 | 75263 | 16: 34933651 |
| 75267 | 16: 35001017 | 75270 | 16: 35137739 | 75272 | 16: 35194830 | 75273 | 16: 35194886 | 75274 | 16: 35194897 |
| 75276 | 16: 35202714 | 75277 | 16: 35234653 | 75278 | 16: 35244272 | 75279 | 16: 35254252 | 75280 | 16: 35290661 |
| 75281 | 16: 35295775 | 75295 | 16: 35792938 | 75302 | 16: 35995859 | 75303 | 16: 36000751 | 75304 | 16: 36004313 |
| 75307 | 16: 36081689 | 75313 | 16: 36290108 | 75314 | 16: 36299734 | 75317 | 16: 36326781 | 75321 | 16: 36365140 |
| 75330 | 16: 36566768 | 75335 | 16: 36697557 | 75336 | 16: 36712618 | 75337 | 16: 36775388 | 75344 | 16: 36945543 |
| 75345 | 16: 36969420 | 75365 | 16: 37448884 | 75366 | 16: 37448890 | 75376 | 16: 37796926 | 75381 | 16: 37913794 |
| 75382 | 16: 37913795 | 75383 | 16: 37913847 | 75384 | 16: 37913888 | 75386 | 16: 37937575 | 75397 | 16: 38107328 |
| 75399 | 16: 38122376 | 75401 | 16: 38137107 | 75402 | 16: 38150887 | 75406 | 16: 38234762 | 75407 | 16: 38244862 |
| 75423 | 16: 38532788 | 75431 | 16: 38885897 | 75458 | 16: 39922839 | 75459 | 16: 39929733 | 75482 | 16: 40598638 |
| 75485 | 16: 40704573 | 75500 | 16: 41266328 | 75513 | 16: 41835712 | 75514 | 16: 41852637 | 75515 | 16: 41881957 |
| 75516 | 16: 41882451 | 75517 | 16: 41882906 | 75518 | 16: 41882957 | 75520 | 16: 41883859 | 75521 | 16: 41884250 |
| 75522 | 16: 41889773 | 75523 | 16: 41890000 | 75524 | 16: 41892823 | 75525 | 16: 41896465 | 75526 | 16: 41898495 |
| 75527 | 16: 41898551 | 75528 | 16: 41908374 | 75529 | 16: 41908542 | 75530 | 16: 41909276 | 75531 | 16: 41909349 |
| 75532 | 16: 41909492 | 75533 | 16: 41909626 | 75534 | 16: 41909729 | 75535 | 16: 41909886 | 75536 | 16: 41929565 |
| 75537 | 16: 41931893 | 75538 | 16: 41931907 | 75539 | 16: 41934619 | 75540 | 16: 41934651 | 75541 | 16: 41934687 |
| 75543 | 16: 41947567 | 75544 | 16: 41964938 | 75554 | 16: 42253482 | 75557 | 16: 42465089 | 75558 | 16: 42547847 |
| 75559 | 16: 42600403 | 75569 | 16: 43061490 | 75575 | 16: 43278476 | 75576 | 16: 43330994 | 75578 | 16: 43398900 |
| 75583 | 16: 43745014 | 75593 | 16: 44139005 | 75604 | 16: 44403647 | 75606 | 16: 44524286 | 75609 | 16: 44660436 |
| 75610 | 16: 44759709 | 75625 | 16: 45375047 | 75641 | 16: 46123551 | 75647 | 16: 46250903 | 75649 | 16: 46275222 |
| 75650 | 16: 46286961 | 75651 | 16: 46287045 | 75658 | 16: 46502252 | 75665 | 16: 46683276 | 75671 | 16: 46787853 |
| 75672 | 16: 46830087 | 75680 | 16: 47040946 | 75682 | 16: 47083406 | 75684 | 16: 47094623 | 75685 | 16: 47120517 |
| 75689 | 16: 47196401 | 75697 | 16: 47379730 | 75698 | 16: 47379738 | 75702 | 16: 47529414 | 75709 | 16: 47649083 |
| 75710 | 16: 47653055 | 75711 | 16: 47658993 | 75719 | 16: 47876641 | 75748 | 16: 48471006 | 75749 | 16: 48471012 |
| 75750 | 16: 48471070 | 75767 | 16: 48815391 | 75788 | 16: 49290662 | 75798 | 16: 49608069 | 75800 | 16: 49640815 |
| 75804 | 16: 49736666 | 75816 | 16: 50023350 | 75856 | 16: 51315511 | 75862 | 16: 51572737 | 75866 | 16: 51638935 |
| 75870 | 16: 51814662 | 75871 | 16: 51818542 | 75872 | 16: 51823133 | 75873 | 16: 51827521 | 75874 | 16: 51828907 |
| 75875 | 16: 51830294 | 75876 | 16: 51830796 | 75877 | 16: 51841289 | 75879 | 16: 51854505 | 75881 | 16: 51899632 |
| 75889 | 16: 52062903 | 75892 | 16: 52124016 | 75896 | 16: 52189080 | 75897 | 16: 52203550 | 75905 | 16: 52391190 |
| 75906 | 16: 52391213 | 75907 | 16: 52401546 | 75908 | 16: 52413488 | 75911 | 16: 52492743 | 75920 | 16: 52664549 |
| 75922 | 16: 52715395 | 75933 | 16: 53089976 | 75945 | 16: 53432925 | 75951 | 16: 53742380 | 75952 | 16: 53742397 |
| 75953 | 16: 53742405 | 75954 | 16: 53742482 | 75960 | 16: 53807690 | 75962 | 16: 53819240 | 75971 | 16: 54022291 |
| 75972 | 16: 54037567 | 75973 | 16: 54047358 | 76003 | 16: 54893701 | 76004 | 16: 54916866 | 76005 | 16: 54917648 |
| 76008 | 16: 54974984 | 76009 | 16: 54975864 | 76010 | 16: 55000694 | 76011 | 16: 55015703 | 76012 | 16: 55021774 |
| 76013 | 16: 55109042 | 76032 | 16: 55624726 | 76041 | 16: 55875606 | 76053 | 16: 56173257 | 76055 | 16: 56193959 |
| 76056 | 16: 56206450 | 76066 | 16: 56367576 | 76067 | 16: 56372228 | 76069 | 16: 56383292 | 76072 | 16: 56444611 |
| 76077 | 16: 56546719 | 76080 | 16: 56571482 | 76081 | 16: 56571485 | 76082 | 16: 56571494 | 76089 | 16: 56837687 |
| 76096 | 16: 57122424 | 76100 | 16: 57326320 | 76101 | 16: 57329788 | 76113 | 16: 57586740 | 76114 | 16: 57608446 |
| 76126 | 16: 57873879 | 76129 | 16: 58011779 | 76130 | 16: 58012586 | 76146 | 16: 58359793 | 76156 | 16: 58464078 |
| 76158 | 16: 58524073 | 76175 | 16: 58904562 | 76176 | 16: 58904595 | 76199 | 16: 59323799 | 76201 | 16: 59330153 |
| 76211 | 16: 59665282 | 76212 | 16: 59689730 | 76213 | 16: 59689802 | 76222 | 16: 59946371 | 76231 | 16: 60201651 |
| 76233 | 16: 60224702 | 76234 | 16: 60224712 | 76236 | 16: 60251070 | 76237 | 16: 60253242 | 76238 | 16: 60254578 |
| 76240 | 16: 60311442 | 76241 | 16: 60322616 | 76242 | 16: 60322634 | 76245 | 16: 60357272 | 76247 | 16: 60391026 |
| 76251 | 16: 60558830 | 76258 | 16: 60784981 | 76274 | 16: 61120937 | 76279 | 16: 61322631 | 76285 | 16: 61383011 |
| 76286 | 16: 61387337 | 76301 | 16: 61731673 | 76306 | 16: 61957053 | 76312 | 16: 62071218 | 76313 | 16: 62072068 |
| 76314 | 16: 62072151 | 76315 | 16: 62072468 | 76316 | 16: 62077343 | 76318 | 16: 62094667 | 76319 | 16: 62094728 |
| 76327 | 16: 62245438 | 76328 | 16: 62267314 | 76329 | 16: 62275255 | 76335 | 16: 62455462 | 76343 | 16: 62633633 |
| 76348 | 16: 62852449 | 76349 | 16: 62856876 | 76354 | 16: 62916406 | 76356 | 16: 62926757 | 76369 | 16: 63226438 |
| 76370 | 16: 63226475 | 76371 | 16: 63252161 | 76372 | 16: 63264840 | 76374 | 16: 63283115 | 76376 | 16: 63353590 |
| 76382 | 16: 63498489 | 76389 | 16: 63665761 | 76391 | 16: 63691003 | 76395 | 16: 63806226 | 76397 | 16: 63832124 |
| 76403 | 16: 63910273 | 76404 | 16: 63918137 | 76406 | 16: 63985213 | 76413 | 16: 64162147 | 76421 | 16: 64386831 |
| 76426 | 16: 64477575 | 76427 | 16: 64478870 | 76428 | 16: 64480600 | 76429 | 16: 64482426 | 76430 | 16: 64483727 |
| 76431 | 16: 64486623 | 76432 | 16: 64488080 | 76434 | 16: 64560495 | 76437 | 16: 64585141 | 76438 | 16: 64587028 |
| 76439 | 16: 64599607 | 76447 | 16: 64716072 | 76457 | 16: 64911089 | 76458 | 16: 64915512 | 76459 | 16: 64917512 |
| 76460 | 16: 64917539 | 76461 | 16: 64920273 | 76462 | 16: 64924645 | 76463 | 16: 64945487 | 76464 | 16: 64947395 |
| 76466 | 16: 64979912 | 76467 | 16: 64979914 | 76468 | 16: 64979920 | 76469 | 16: 64980083 | 76473 | 16: 65100643 |
| 76479 | 16: 65256823 | 76480 | 16: 65263949 | 76483 | 16: 65320518 | 76490 | 16: 65492703 | 76491 | 16: 65500984 |
| 76492 | 16: 65508159 | 76493 | 16: 65511465 | 76494 | 16: 65514812 | 76495 | 16: 65518974 | 76496 | 16: 65521306 |
| 76505 | 16: 65841167 | 76512 | 16: 66020681 | 76515 | 16: 66100353 | 76517 | 16: 66121444 | 76521 | 16: 66169638 |
| 76523 | 16: 66271884 | 76532 | 16: 66520379 | 76533 | 16: 66528712 | 76536 | 16: 66553762 | 76538 | 16: 66566651 |
| 76550 | 16: 66804665 | 76551 | 16: 66825677 | 76553 | 16: 66859242 | 76555 | 16: 66896436 | 76557 | 16: 66919773 |
| 76558 | 16: 66919950 | 76564 | 16: 67050756 | 76565 | 16: 67108343 | 76571 | 16: 67259430 | 76573 | 16: 67296635 |
| 76580 | 16: 67413442 | 76581 | 16: 67414503 | 76582 | 16: 67414576 | 76583 | 16: 67414591 | 76584 | 16: 67418245 |
| 76588 | 16: 67495932 | 76599 | 16: 67610069 | 76610 | 16: 67976663 | 76611 | 16: 67981588 | 76626 | 16: 68435498 |
| 76627 | 16: 68435550 | 76636 | 16: 68624997 | 76652 | 16: 69066498 | 76653 | 16: 69066625 | 76656 | 16: 69080402 |
| 76657 | 16: 69086868 | 76660 | 16: 69129862 | 76665 | 16: 69230127 | 76671 | 16: 69350387 | 76679 | 16: 69497807 |
| 76680 | 16: 69501212 | 76681 | 16: 69541904 | 76685 | 16: 69607606 | 76688 | 16: 69676831 | 76689 | 16: 69704477 |
| 76692 | 16: 69802000 | 76719 | 16: 70380777 | 76720 | 16: 70381368 | 76730 | 16: 70587239 | 76731 | 16: 70590791 |
| 76732 | 16: 70590919 | 76733 | 16: 70608188 | 76744 | 16: 70871474 | 76754 | 16: 71132906 | 76759 | 16: 71385575 |
| 76762 | 16: 71432921 | 76766 | 16: 71483613 | 76771 | 16: 71716120 | 76772 | 16: 71732153 | 76777 | 16: 71834818 |
| 76784 | 16: 71964464 | 76785 | 16: 71974676 | 76792 | 16: 72212208 | 76797 | 16: 72370528 | 76798 | 16: 72370550 |
| 76799 | 16: 72370562 | 76800 | 16: 72370636 | 76803 | 16: 72382117 | 76804 | 16: 72382163 | 76805 | 16: 72382236 |
| 76806 | 16: 72382280 | 76808 | 16: 72394792 | 76811 | 16: 72421917 | 76813 | 16: 72440326 | 76814 | 16: 72473155 |
| 76815 | 16: 72473197 | 76821 | 16: 72619958 | 76823 | 16: 72642068 | 76826 | 16: 72684703 | 76828 | 16: 72684778 |
| 76829 | 16: 72684811 | 76831 | 16: 72698852 | 76832 | 16: 72700502 | 76833 | 16: 72701289 | 76848 | 16: 73201335 |
| 76853 | 16: 73303554 | 76860 | 16: 73466388 | 76862 | 16: 73504205 | 76865 | 16: 73518067 | 76866 | 16: 73534249 |
| 76868 | 16: 73563070 | 76869 | 16: 73563134 | 76871 | 16: 73583302 | 76873 | 16: 73591363 | 76874 | 16: 73597712 |
| 76876 | 16: 73599978 | 76877 | 16: 73607828 | 76878 | 16: 73622925 | 76879 | 16: 73694168 | 76880 | 16: 73694195 |
| 76881 | 16: 73694803 | 76882 | 16: 73697167 | 76883 | 16: 73697204 | 76884 | 16: 73697206 | 76885 | 16: 73697258 |
| 76886 | 16: 73697700 | 76887 | 16: 73699147 | 76889 | 16: 73715301 | 76892 | 16: 73750079 | 76909 | 16: 74262736 |
| 76910 | 16: 74267964 | 76924 | 16: 74548368 | 76931 | 16: 74775935 | 76936 | 16: 74853499 | 76945 | 16: 75005400 |
| 76946 | 16: 75005468 | 76948 | 16: 75032047 | 76953 | 16: 75118546 | 76954 | 16: 75132927 | 76955 | 16: 75142495 |
| 76956 | 16: 75143544 | 76957 | 16: 75153428 | 76959 | 16: 75202467 | 76960 | 16: 75217027 | 76963 | 16: 75248051 |
| 76965 | 16: 75255731 | 76966 | 16: 75256528 | 76967 | 16: 75273493 | 76971 | 16: 75346062 | 76978 | 16: 75476985 |
| 76979 | 16: 75477188 | 76980 | 16: 75487044 | 76986 | 16: 75656899 | 76991 | 16: 75736008 | 76996 | 16: 75817486 |
| 77008 | 16: 76064461 | 77010 | 16: 76082386 | 77016 | 16: 76264493 | 77020 | 16: 76316949 | 77023 | 16: 76382298 |
| 77025 | 16: 76417156 | 77026 | 16: 76424214 | 77029 | 16: 76490659 | 77034 | 16: 76550668 | 77035 | 16: 76561525 |
| 77038 | 16: 76632577 | 77039 | 16: 76645921 | 77044 | 16: 76724147 | 77047 | 16: 76749641 | 77049 | 16: 76810446 |
| 77050 | 16: 76898157 | 77053 | 16: 77026179 | 77066 | 16: 77196110 | 77075 | 16: 77345710 | 77076 | 16: 77345711 |
| 77086 | 16: 77672463 | 77088 | 16: 77700431 | 77133 | 16: 78730933 | 77136 | 16: 78794726 | 77146 | 16: 78936706 |
| 77147 | 16: 78936848 | 77148 | 16: 78936900 | 77162 | 16: 79072375 | 77170 | 16: 79207667 | 77171 | 16: 79214144 |
| 77172 | 16: 79214863 | 77173 | 16: 79214898 | 77174 | 16: 79216805 | 77175 | 16: 79216824 | 77176 | 16: 79216879 |
| 77177 | 16: 79217281 | 77178 | 16: 79246839 | 77179 | 16: 79247022 | 77180 | 16: 79248848 | 77181 | 16: 79252434 |
| 77182 | 16: 79252455 | 77183 | 16: 79253093 | 77184 | 16: 79253212 | 77185 | 16: 79253954 | 77186 | 16: 79256053 |
| 77187 | 16: 79258337 | 77188 | 16: 79258375 | 77189 | 16: 79259607 | 77190 | 16: 79259608 | 77191 | 16: 79259624 |
| 77192 | 16: 79259664 | 77193 | 16: 79259693 | 77194 | 16: 79264907 | 77195 | 16: 79264964 | 77196 | 16: 79265514 |
| 77197 | 16: 79265600 | 77199 | 16: 79316462 | 77203 | 16: 79347181 | 77206 | 16: 79436693 | 77207 | 16: 79449942 |
| 77208 | 16: 79458180 | 77211 | 16: 79513573 | 77212 | 16: 79514562 | 77213 | 16: 79526906 | 77215 | 16: 79554621 |
| 77216 | 16: 79563778 | 77217 | 16: 79626644 | 77219 | 16: 79659075 | 77223 | 16: 79862681 | 77224 | 16: 79881468 |
| 77225 | 16: 79889426 | 77228 | 16: 80019106 | 77231 | 16: 80220495 | 77234 | 16: 80431031 | 77240 | 16: 80869601 |
| 77259 | 17: 425858 | 77280 | 17: 1125457 | 77282 | 17: 1162137 | 77283 | 17: 1162139 | 77284 | 17: 1162184 |
| 77285 | 17: 1162199 | 77286 | 17: 1162200 | 77300 | 17: 1367292 | 77306 | 17: 1539162 | 77316 | 17: 1782217 |
| 77338 | 17: 2449957 | 77357 | 17: 2912361 | 77367 | 17: 3145731 | 77376 | 17: 3382377 | 77391 | 17: 3546676 |
| 77399 | 17: 3801647 | 77403 | 17: 3941038 | 77406 | 17: 3948766 | 77407 | 17: 3948856 | 77408 | 17: 3950488 |
| 77409 | 17: 3950503 | 77410 | 17: 3979701 | 77413 | 17: 4046782 | 77419 | 17: 4192418 | 77424 | 17: 4272755 |
| 77426 | 17: 4298012 | 77432 | 17: 4394112 | 77442 | 17: 4571004 | 77447 | 17: 4633530 | 77448 | 17: 4657147 |
| 77453 | 17: 4721455 | 77454 | 17: 4721515 | 77456 | 17: 4770586 | 77460 | 17: 4835673 | 77465 | 17: 4973219 |
| 77479 | 17: 5317539 | 77481 | 17: 5375833 | 77482 | 17: 5398061 | 77490 | 17: 5679415 | 77491 | 17: 5679423 |
| 77492 | 17: 5679424 | 77493 | 17: 5679464 | 77494 | 17: 5679532 | 77496 | 17: 5728456 | 77512 | 17: 6078923 |
| 77513 | 17: 6107701 | 77516 | 17: 6161274 | 77517 | 17: 6198963 | 77518 | 17: 6205937 | 77520 | 17: 6220260 |
| 77521 | 17: 6229560 | 77522 | 17: 6258944 | 77526 | 17: 6341387 | 77532 | 17: 6459287 | 77533 | 17: 6491029 |
| 77537 | 17: 6552131 | 77539 | 17: 6614404 | 77540 | 17: 6625470 | 77541 | 17: 6626902 | 77544 | 17: 6698968 |
| 77545 | 17: 6701688 | 77546 | 17: 6707615 | 77547 | 17: 6710298 | 77548 | 17: 6715516 | 77554 | 17: 6811535 |
| 77555 | 17: 6819346 | 77559 | 17: 6940926 | 77560 | 17: 6984368 | 77561 | 17: 6985910 | 77563 | 17: 7027194 |
| 77588 | 17: 7507100 | 77596 | 17: 7694458 | 77598 | 17: 7697113 | 77602 | 17: 7770098 | 77612 | 17: 8012088 |
| 77613 | 17: 8012177 | 77621 | 17: 8235574 | 77637 | 17: 8588779 | 77661 | 17: 9138287 | 77674 | 17: 9441496 |
| 77683 | 17: 9768434 | 77684 | 17: 9768476 | 77688 | 17: 9888649 | 77695 | 17: 9978114 | 77696 | 17: 10024882 |
| 77698 | 17: 10050562 | 77700 | 17: 10077655 | 77702 | 17: 10100298 | 77705 | 17: 10110670 | 77709 | 17: 10169816 |
| 77710 | 17: 10176209 | 77717 | 17: 10353846 | 77720 | 17: 10489157 | 77730 | 17: 10813567 | 77731 | 17: 10814317 |
| 77749 | 17: 11330319 | 77765 | 17: 11778925 | 77771 | 17: 11817119 | 77780 | 17: 12090766 | 77797 | 17: 12362004 |
| 77814 | 17: 12766769 | 77823 | 17: 12938879 | 77828 | 17: 13174673 | 77832 | 17: 13361331 | 77837 | 17: 13490837 |
| 77838 | 17: 13490864 | 77849 | 17: 13820980 | 77850 | 17: 13883525 | 77853 | 17: 13941930 | 77865 | 17: 14194666 |
| 77866 | 17: 14208519 | 77867 | 17: 14212322 | 77868 | 17: 14214868 | 77870 | 17: 14226993 | 77871 | 17: 14233961 |
| 77874 | 17: 14255518 | 77875 | 17: 14257411 | 77876 | 17: 14258646 | 77877 | 17: 14265993 | 77878 | 17: 14267278 |
| 77879 | 17: 14268573 | 77885 | 17: 14447098 | 77889 | 17: 14526830 | 77892 | 17: 14548325 | 77897 | 17: 14641198 |
| 77912 | 17: 14905289 | 77913 | 17: 14921802 | 77920 | 17: 15088817 | 77925 | 17: 15159023 | 77929 | 17: 15328788 |
| 77931 | 17: 15328888 | 77937 | 17: 15455174 | 77940 | 17: 15527126 | 77948 | 17: 15652225 | 77952 | 17: 15705665 |
| 77959 | 17: 15911943 | 77967 | 17: 16144638 | 77970 | 17: 16239054 | 77981 | 17: 16560132 | 77983 | 17: 16659063 |
| 77984 | 17: 16684179 | 77989 | 17: 16808806 | 77990 | 17: 16833027 | 78001 | 17: 17127455 | 78002 | 17: 17127599 |
| 78004 | 17: 17132830 | 78007 | 17: 17224074 | 78021 | 17: 17389673 | 78028 | 17: 17475047 | 78029 | 17: 17475831 |
| 78048 | 17: 17963586 | 78050 | 17: 17992691 | 78054 | 17: 18028087 | 78057 | 17: 18062413 | 78060 | 17: 18161738 |
| 78064 | 17: 18284618 | 78087 | 17: 18873882 | 78092 | 17: 18900848 | 78094 | 17: 18961495 | 78102 | 17: 19217997 |
| 78130 | 17: 19581434 | 78135 | 17: 19786181 | 78141 | 17: 19961480 | 78142 | 17: 19965877 | 78144 | 17: 20018582 |
| 78149 | 17: 20111214 | 78152 | 17: 20217310 | 78157 | 17: 20333021 | 78227 | 17: 22641428 | 78231 | 17: 22853179 |
| 78245 | 17: 23369245 | 78266 | 17: 24189137 | 78275 | 17: 24523000 | 78285 | 17: 24792731 | 78294 | 17: 25041571 |
| 78312 | 17: 25581271 | 78323 | 17: 25977654 | 78324 | 17: 25977693 | 78325 | 17: 25977723 | 78326 | 17: 25977762 |
| 78369 | 17: 26883751 | 78370 | 17: 26915514 | 78393 | 17: 27503090 | 78394 | 17: 27559967 | 78399 | 17: 27653769 |
| 78408 | 17: 27921810 | 78413 | 17: 28059892 | 78427 | 17: 28334342 | 78428 | 17: 28334349 | 78439 | 17: 28702389 |
| 78464 | 17: 29480103 | 78469 | 17: 29662529 | 78478 | 17: 29993789 | 78489 | 17: 30232347 | 78490 | 17: 30304435 |
| 78497 | 17: 30487913 | 78510 | 17: 30674204 | 78511 | 17: 30696794 | 78512 | 17: 30709277 | 78516 | 17: 30799626 |
| 78524 | 17: 30996169 | 78544 | 17: 31562261 | 78549 | 17: 31735789 | 78551 | 17: 31735957 | 78561 | 17: 31998920 |
| 78562 | 17: 32030137 | 78571 | 17: 32394529 | 78572 | 17: 32394568 | 78573 | 17: 32403823 | 78574 | 17: 32403842 |
| 78594 | 17: 32777788 | 78599 | 17: 32874912 | 78618 | 17: 33384490 | 78641 | 17: 33842930 | 78655 | 17: 34228544 |
| 78665 | 17: 34544058 | 78678 | 17: 34902455 | 78685 | 17: 35149936 | 78686 | 17: 35186932 | 78694 | 17: 35488740 |
| 78704 | 17: 35746205 | 78706 | 17: 35787842 | 78707 | 17: 35799086 | 78709 | 17: 35857580 | 78712 | 17: 35940925 |
| 78719 | 17: 36144107 | 78728 | 17: 36520470 | 78737 | 17: 36882303 | 78781 | 17: 38220984 | 78810 | 17: 39276967 |
| 78812 | 17: 39326538 | 78837 | 17: 39843444 | 78842 | 17: 39882804 | 78852 | 17: 39935546 | 78860 | 17: 40113609 |
| 78865 | 17: 40279234 | 78867 | 17: 40310128 | 78868 | 17: 40314686 | 78879 | 17: 40566975 | 78883 | 17: 40575300 |
| 78886 | 17: 40597293 | 78894 | 17: 40765558 | 78897 | 17: 40880376 | 78898 | 17: 40882503 | 78905 | 17: 41002973 |
| 78932 | 17: 41864826 | 78941 | 17: 42170894 | 78942 | 17: 42186823 | 78943 | 17: 42188310 | 78946 | 17: 42229273 |
| 78954 | 17: 42393090 | 78956 | 17: 42415227 | 78970 | 17: 42715046 | 78976 | 17: 42768963 | 78978 | 17: 42821182 |
| 78982 | 17: 42891704 | 78983 | 17: 42923614 | 78997 | 17: 43357874 | 79000 | 17: 43436343 | 79003 | 17: 43553874 |
| 79006 | 17: 43577102 | 79007 | 17: 43577588 | 79008 | 17: 43594706 | 79011 | 17: 43609022 | 79012 | 17: 43623722 |
| 79029 | 17: 44194890 | 79032 | 17: 44224761 | 79040 | 17: 44531668 | 79046 | 17: 44677660 | 79052 | 17: 44819760 |
| 79058 | 17: 44927880 | 79063 | 17: 45086949 | 79064 | 17: 45091135 | 79066 | 17: 45172949 | 79068 | 17: 45178502 |
| 79069 | 17: 45178540 | 79070 | 17: 45180519 | 79071 | 17: 45180612 | 79072 | 17: 45181370 | 79073 | 17: 45182722 |
| 79091 | 17: 45616057 | 79094 | 17: 45734727 | 79112 | 17: 46168639 | 79113 | 17: 46171496 | 79127 | 17: 46408391 |
| 79135 | 17: 46615898 | 79136 | 17: 46624459 | 79142 | 17: 46676910 | 79187 | 17: 47856534 | 79215 | 17: 48637182 |
| 79244 | 17: 49321412 | 79268 | 17: 49848060 | 79273 | 17: 49923094 | 79285 | 17: 50262075 | 79287 | 17: 50270777 |
| 79288 | 17: 50274476 | 79315 | 17: 50731825 | 79316 | 17: 50769132 | 79319 | 17: 50841533 | 79320 | 17: 50864647 |
| 79322 | 17: 50902320 | 79326 | 17: 50955494 | 79327 | 17: 50959261 | 79339 | 17: 51345327 | 79342 | 17: 51469617 |
| 79343 | 17: 51470914 | 79346 | 17: 51529156 | 79348 | 17: 51546917 | 79355 | 17: 51627470 | 79359 | 17: 51685973 |
| 79360 | 17: 51689778 | 79364 | 17: 51762211 | 79366 | 17: 51792442 | 79368 | 17: 51813934 | 79375 | 17: 51958814 |
| 79377 | 17: 51959782 | 79379 | 17: 52027381 | 79395 | 17: 52307367 | 79403 | 17: 52573801 | 79414 | 17: 52988658 |
| 79417 | 17: 53020835 | 79423 | 17: 53275351 | 79428 | 17: 53388401 | 79430 | 17: 53390888 | 79431 | 17: 53448341 |
| 79433 | 17: 53470903 | 79435 | 17: 53532750 | 79447 | 17: 53919113 | 79454 | 17: 54025669 | 79455 | 17: 54029457 |
| 79456 | 17: 54030902 | 79457 | 17: 54033380 | 79458 | 17: 54062683 | 79459 | 17: 54122851 | 79461 | 17: 54128175 |
| 79465 | 17: 54163192 | 79468 | 17: 54264980 | 79469 | 17: 54267012 | 79470 | 17: 54310765 | 79483 | 17: 54668554 |
| 79489 | 17: 54807887 | 79499 | 17: 54895464 | 79505 | 17: 55074569 | 79506 | 17: 55081286 | 79507 | 17: 55082228 |
| 79508 | 17: 55105646 | 79509 | 17: 55105686 | 79512 | 17: 55385614 | 79517 | 17: 55484565 | 79521 | 17: 55592718 |
| 79523 | 17: 55665541 | 79524 | 17: 55716549 | 79526 | 17: 55752066 | 79532 | 17: 55876873 | 79537 | 17: 56011560 |
| 79548 | 17: 56279452 | 79549 | 17: 56281095 | 79560 | 17: 56566250 | 79564 | 17: 56651078 | 79565 | 17: 56710101 |
| 79571 | 17: 56976866 | 79576 | 17: 57116303 | 79583 | 17: 57285859 | 79591 | 17: 57472146 | 79598 | 17: 57643506 |
| 79601 | 17: 57716356 | 79602 | 17: 57730355 | 79603 | 17: 57731200 | 79610 | 17: 57797646 | 79612 | 17: 57831569 |
| 79623 | 17: 58093779 | 79625 | 17: 58112817 | 79626 | 17: 58130419 | 79627 | 17: 58141075 | 79634 | 17: 58304066 |
| 79665 | 17: 58556801 | 79670 | 17: 58775371 | 79679 | 17: 59008052 | 79688 | 17: 59157020 | 79690 | 17: 59215735 |
| 79700 | 17: 59384904 | 79702 | 17: 59397574 | 79710 | 17: 59604994 | 79714 | 17: 59716548 | 79719 | 17: 59839482 |
| 79730 | 17: 60067619 | 79731 | 17: 60090877 | 79732 | 17: 60090928 | 79733 | 17: 60097662 | 79743 | 17: 60248908 |
| 79751 | 17: 60373041 | 79752 | 17: 60374859 | 79753 | 17: 60395890 | 79754 | 17: 60395994 | 79760 | 17: 60500390 |
| 79771 | 17: 60710033 | 79772 | 17: 60710035 | 79773 | 17: 60710040 | 79774 | 17: 60710101 | 79779 | 17: 60795229 |
| 79780 | 17: 60795274 | 79781 | 17: 60795346 | 79784 | 17: 60843399 | 79807 | 17: 61141160 | 79812 | 17: 61202979 |
| 79814 | 17: 61208274 | 79815 | 17: 61208429 | 79816 | 17: 61237780 | 79819 | 17: 61316287 | 79822 | 17: 61370873 |
| 79828 | 17: 61499579 | 79829 | 17: 61499616 | 79842 | 17: 61892683 | 79846 | 17: 61971343 | 79850 | 17: 62029219 |
| 79858 | 17: 62310603 | 79866 | 17: 62556427 | 79874 | 17: 62749389 | 79882 | 17: 62937635 | 79884 | 17: 62967008 |
| 79886 | 17: 63060456 | 79890 | 17: 63193363 | 79904 | 17: 63343949 | 79909 | 17: 63512971 | 79912 | 17: 63562509 |
| 79916 | 17: 63612425 | 79917 | 17: 63613044 | 79918 | 17: 63614883 | 79919 | 17: 63615321 | 79920 | 17: 63615338 |
| 79921 | 17: 63619368 | 79922 | 17: 63619438 | 79923 | 17: 63619955 | 79926 | 17: 63623292 | 79927 | 17: 63624426 |
| 79933 | 17: 63696661 | 79937 | 17: 63745078 | 79938 | 17: 63765593 | 79946 | 17: 63909068 | 79947 | 17: 63911823 |
| 79948 | 17: 63914506 | 79949 | 17: 63924591 | 79953 | 17: 64073618 | 79954 | 17: 64077337 | 79955 | 17: 64079380 |
| 79957 | 17: 64164363 | 79959 | 17: 64233930 | 79963 | 17: 64320209 | 79964 | 17: 64364133 | 79965 | 17: 64376618 |
| 79966 | 17: 64381714 | 79967 | 17: 64390370 | 79968 | 17: 64391421 | 79969 | 17: 64392375 | 79971 | 17: 64400070 |
| 79975 | 17: 64543414 | 79983 | 17: 64868669 | 79986 | 17: 64936914 | 80004 | 17: 65250897 | 80005 | 17: 65252978 |
| 80007 | 17: 65269001 | 80009 | 17: 65311178 | 80015 | 17: 65495113 | 80016 | 17: 65515359 | 80028 | 17: 65711335 |
| 80046 | 17: 66084982 | 80048 | 17: 66098132 | 80091 | 17: 66926217 | 80093 | 17: 66962321 | 80094 | 17: 66963375 |
| 80095 | 17: 66964444 | 80096 | 17: 66980228 | 80097 | 17: 66981196 | 80098 | 17: 66983892 | 80099 | 17: 66987390 |
| 80100 | 17: 66989033 | 80101 | 17: 66991682 | 80102 | 17: 66993583 | 80117 | 17: 67452220 | 80120 | 17: 67493854 |
| 80123 | 17: 67576683 | 80133 | 17: 67991825 | 80143 | 17: 68190606 | 80145 | 17: 68235847 | 80146 | 17: 68237141 |
| 80152 | 17: 68336966 | 80154 | 17: 68390649 | 80156 | 17: 68404948 | 80165 | 17: 68610408 | 80169 | 17: 68711761 |
| 80177 | 17: 68924124 | 80186 | 17: 69025532 | 80187 | 17: 69025781 | 80191 | 17: 69104813 | 80200 | 17: 69235524 |
| 80201 | 17: 69244183 | 80208 | 17: 69526999 | 80209 | 17: 69557728 | 80210 | 17: 69558386 | 80221 | 17: 69723039 |
| 80226 | 17: 69823676 | 80236 | 17: 70089184 | 80242 | 17: 70183712 | 80266 | 17: 70611985 | 80267 | 17: 70615923 |
| 80268 | 17: 70617839 | 80269 | 17: 70618022 | 80270 | 17: 70618023 | 80271 | 17: 70619770 | 80272 | 17: 70621442 |
| 80294 | 17: 71216081 | 80295 | 17: 71224018 | 80297 | 17: 71275992 | 80298 | 17: 71276089 | 80299 | 17: 71276886 |
| 80300 | 17: 71276966 | 80301 | 17: 71283804 | 80306 | 17: 71441658 | 80309 | 17: 71512874 | 80310 | 17: 71518391 |
| 80311 | 17: 71528096 | 80312 | 17: 71528119 | 80313 | 17: 71528120 | 80314 | 17: 71528861 | 80315 | 17: 71528882 |
| 80316 | 17: 71528942 | 80324 | 17: 71695918 | 80325 | 17: 71702829 | 80328 | 17: 71812939 | 80339 | 17: 71951302 |
| 80349 | 17: 72204450 | 80356 | 17: 72342109 | 80358 | 17: 72363855 | 80360 | 17: 72369402 | 80362 | 17: 72398155 |
| 80364 | 17: 72408383 | 80365 | 17: 72445427 | 80366 | 17: 72447709 | 80367 | 17: 72457694 | 80369 | 17: 72468245 |
| 80370 | 17: 72498960 | 80374 | 17: 72555058 | 80384 | 17: 72883784 | 80386 | 17: 72916533 | 80391 | 17: 73036168 |
| 80411 | 18: 868580 | 80413 | 18: 911112 | 80414 | 18: 911196 | 80415 | 18: 932024 | 80416 | 18: 932036 |
| 80417 | 18: 932132 | 80431 | 18: 1178922 | 80435 | 18: 1215919 | 80436 | 18: 1269485 | 80437 | 18: 1269511 |
| 80438 | 18: 1269570 | 80452 | 18: 1553979 | 80453 | 18: 1565391 | 80460 | 18: 1658820 | 80467 | 18: 1733905 |
| 80468 | 18: 1734779 | 80469 | 18: 1737507 | 80470 | 18: 1739009 | 80471 | 18: 1741139 | 80472 | 18: 1742546 |
| 80474 | 18: 1743718 | 80475 | 18: 1744700 | 80476 | 18: 1745880 | 80477 | 18: 1746570 | 80478 | 18: 1747824 |
| 80479 | 18: 1749671 | 80480 | 18: 1751021 | 80481 | 18: 1752037 | 80482 | 18: 1753160 | 80483 | 18: 1754297 |
| 80484 | 18: 1754989 | 80485 | 18: 1755466 | 80486 | 18: 1758653 | 80487 | 18: 1759177 | 80488 | 18: 1759407 |
| 80489 | 18: 1759775 | 80490 | 18: 1760828 | 80491 | 18: 1761351 | 80492 | 18: 1761856 | 80493 | 18: 1764061 |
| 80494 | 18: 1765063 | 80495 | 18: 1765811 | 80496 | 18: 1766413 | 80497 | 18: 1766979 | 80498 | 18: 1780698 |
| 80499 | 18: 1799373 | 80503 | 18: 1821333 | 80515 | 18: 2132264 | 80519 | 18: 2189010 | 80525 | 18: 2310539 |
| 80529 | 18: 2407203 | 80532 | 18: 2547395 | 80535 | 18: 2627721 | 80548 | 18: 2801078 | 80549 | 18: 2824948 |
| 80553 | 18: 2893572 | 80559 | 18: 2948533 | 80590 | 18: 3732736 | 80593 | 18: 3803407 | 80599 | 18: 3918400 |
| 80604 | 18: 4117718 | 80610 | 18: 4175893 | 80622 | 18: 4456924 | 80623 | 18: 4458325 | 80652 | 18: 5105107 |
| 80657 | 18: 5261361 | 80658 | 18: 5303405 | 80667 | 18: 5381034 | 80668 | 18: 5392289 | 80686 | 18: 5796418 |
| 80695 | 18: 6075338 | 80699 | 18: 6140925 | 80700 | 18: 6150654 | 80702 | 18: 6155426 | 80708 | 18: 6179950 |
| 80710 | 18: 6195750 | 80717 | 18: 6307757 | 80732 | 18: 6411938 | 80733 | 18: 6414217 | 80734 | 18: 6434042 |
| 80736 | 18: 6473072 | 80742 | 18: 6606486 | 80756 | 18: 6904961 | 80759 | 18: 6952523 | 80775 | 18: 7393588 |
| 80782 | 18: 7571670 | 80783 | 18: 7571744 | 80784 | 18: 7571766 | 80790 | 18: 7722433 | 80791 | 18: 7725494 |
| 80792 | 18: 7759211 | 80794 | 18: 7785017 | 80795 | 18: 7806089 | 80798 | 18: 7857634 | 80809 | 18: 8151586 |
| 80814 | 18: 8174600 | 80816 | 18: 8217290 | 80817 | 18: 8219384 | 80818 | 18: 8219385 | 80819 | 18: 8219497 |
| 80836 | 18: 8556730 | 80847 | 18: 8895607 | 80850 | 18: 8930714 | 80868 | 18: 9094422 | 80869 | 18: 9121273 |
| 80871 | 18: 9155492 | 80873 | 18: 9160707 | 80877 | 18: 9224812 | 80879 | 18: 9281528 | 80891 | 18: 9500208 |
| 80893 | 18: 9514100 | 80897 | 18: 9541606 | 80898 | 18: 9560416 | 80903 | 18: 9622228 | 80913 | 18: 9939597 |
| 80915 | 18: 9939685 | 80920 | 18: 10053603 | 80937 | 18: 10466956 | 80938 | 18: 10467016 | 80939 | 18: 10474781 |
| 80940 | 18: 10474891 | 80944 | 18: 10554759 | 80945 | 18: 10555268 | 80949 | 18: 10623982 | 80954 | 18: 10638844 |
| 80955 | 18: 10661516 | 80959 | 18: 10775715 | 80966 | 18: 10924943 | 80970 | 18: 11032803 | 80972 | 18: 11076163 |
| 80975 | 18: 11143812 | 80978 | 18: 11177661 | 80979 | 18: 11189421 | 80982 | 18: 11197228 | 80985 | 18: 11315060 |
| 80986 | 18: 11322791 | 80987 | 18: 11333644 | 80988 | 18: 11340350 | 80989 | 18: 11349111 | 80990 | 18: 11352571 |
| 80991 | 18: 11362283 | 80992 | 18: 11367578 | 80993 | 18: 11398671 | 80995 | 18: 11499305 | 80996 | 18: 11499360 |
| 80997 | 18: 11589376 | 80999 | 18: 11631258 | 81001 | 18: 11639177 | 81002 | 18: 11640852 | 81004 | 18: 11652518 |
| 81007 | 18: 11719208 | 81026 | 18: 12374145 | 81030 | 18: 12487243 | 81036 | 18: 12677943 | 81058 | 18: 13316111 |
| 81059 | 18: 13322081 | 81060 | 18: 13326301 | 81068 | 18: 13483552 | 81090 | 18: 14035593 | 81092 | 18: 14117250 |
| 81097 | 18: 14266021 | 81104 | 18: 14579399 | 81110 | 18: 14880493 | 81111 | 18: 14918649 | 81121 | 18: 15157858 |
| 81129 | 18: 15352349 | 81130 | 18: 15353723 | 81136 | 18: 15529204 | 81137 | 18: 15532676 | 81138 | 18: 15535125 |
| 81139 | 18: 15537825 | 81142 | 18: 15574419 | 81145 | 18: 15607698 | 81147 | 18: 15643821 | 81149 | 18: 15771762 |
| 81163 | 18: 16246520 | 81164 | 18: 16246606 | 81165 | 18: 16246649 | 81167 | 18: 16294087 | 81170 | 18: 16339724 |
| 81179 | 18: 16491063 | 81186 | 18: 16701247 | 81188 | 18: 16722963 | 81191 | 18: 16855403 | 81194 | 18: 16929518 |
| 81198 | 18: 16960682 | 81205 | 18: 17095383 | 81206 | 18: 17095391 | 81207 | 18: 17095421 | 81208 | 18: 17128188 |
| 81214 | 18: 17231296 | 81219 | 18: 17332696 | 81224 | 18: 17474883 | 81225 | 18: 17474962 | 81246 | 18: 17964994 |
| 81248 | 18: 18015282 | 81259 | 18: 18234827 | 81274 | 18: 18340451 | 81277 | 18: 18455809 | 81281 | 18: 18555382 |
| 81286 | 18: 18634395 | 81288 | 18: 18652008 | 81292 | 18: 18693621 | 81296 | 18: 18741396 | 81297 | 18: 18766595 |
| 81304 | 18: 18991234 | 81308 | 18: 19028089 | 81312 | 18: 19104084 | 81318 | 18: 19279732 | 81319 | 18: 19306933 |
| 81321 | 18: 19321180 | 81328 | 18: 19549103 | 81337 | 18: 19828522 | 81341 | 18: 19884211 | 81346 | 18: 19992495 |
| 81379 | 18: 20783780 | 81396 | 18: 21231861 | 81397 | 18: 21231877 | 81404 | 18: 21429864 | 81405 | 18: 21429866 |
| 81419 | 18: 21805702 | 81422 | 18: 21842921 | 81427 | 18: 21915772 | 81428 | 18: 21944535 | 81429 | 18: 21944650 |
| 81439 | 18: 22075634 | 81459 | 18: 22432351 | 81467 | 18: 22574217 | 81468 | 18: 22581424 | 81470 | 18: 22616518 |
| 81489 | 18: 23048862 | 81515 | 18: 23639968 | 81523 | 18: 23885882 | 81526 | 18: 23978279 | 81530 | 18: 24024754 |
| 81531 | 18: 24028549 | 81532 | 18: 24039248 | 81538 | 18: 24159516 | 81541 | 18: 24233256 | 81547 | 18: 24449226 |
| 81548 | 18: 24467752 | 81551 | 18: 24569333 | 81555 | 18: 24603788 | 81556 | 18: 24605526 | 81557 | 18: 24606504 |
| 81558 | 18: 24609637 | 81559 | 18: 24622318 | 81560 | 18: 24626076 | 81561 | 18: 24627052 | 81562 | 18: 24640038 |
| 81563 | 18: 24646900 | 81564 | 18: 24648311 | 81565 | 18: 24650296 | 81566 | 18: 24653596 | 81567 | 18: 24655887 |
| 81568 | 18: 24658898 | 81569 | 18: 24680546 | 81572 | 18: 24703105 | 81573 | 18: 24711358 | 81581 | 18: 24916840 |
| 81586 | 18: 25087427 | 81594 | 18: 25243259 | 81595 | 18: 25263618 | 81597 | 18: 25320467 | 81603 | 18: 25475882 |
| 81604 | 18: 25507140 | 81608 | 18: 25560734 | 81613 | 18: 25671777 | 81614 | 18: 25693559 | 81617 | 18: 25769838 |
| 81618 | 18: 25772348 | 81619 | 18: 25777563 | 81622 | 18: 25833720 | 81626 | 18: 25944954 | 81638 | 18: 26118698 |
| 81643 | 18: 26252156 | 81648 | 18: 26367037 | 81652 | 18: 26448739 | 81674 | 18: 26911688 | 81683 | 18: 27123603 |
| 81711 | 18: 27938774 | 81712 | 18: 27938830 | 81733 | 18: 28523029 | 81752 | 18: 29298380 | 81753 | 18: 29324758 |
| 81755 | 18: 29354497 | 81756 | 18: 29354560 | 81757 | 18: 29354692 | 81758 | 18: 29356162 | 81759 | 18: 29359726 |
| 81760 | 18: 29359801 | 81765 | 18: 29507594 | 81766 | 18: 29507596 | 81767 | 18: 29507695 | 81768 | 18: 29517248 |
| 81800 | 18: 30444281 | 81812 | 18: 30824413 | 81820 | 18: 31114666 | 81821 | 18: 31161073 | 81829 | 18: 31403926 |
| 81860 | 18: 32193066 | 81865 | 18: 32277972 | 81866 | 18: 32277986 | 81889 | 18: 32769354 | 81892 | 18: 32797023 |
| 81893 | 18: 32809799 | 81909 | 18: 33174459 | 81913 | 18: 33266043 | 81915 | 18: 33332355 | 81928 | 18: 33524126 |
| 81938 | 18: 33726109 | 81942 | 18: 33803131 | 81943 | 18: 33819518 | 81948 | 18: 33891121 | 81950 | 18: 33985539 |
| 81958 | 18: 34273373 | 81960 | 18: 34305936 | 81962 | 18: 34318674 | 81963 | 18: 34340817 | 81967 | 18: 34415049 |
| 81968 | 18: 34460275 | 81969 | 18: 34460342 | 81971 | 18: 34477636 | 81973 | 18: 34507746 | 81976 | 18: 34573297 |
| 81979 | 18: 34674118 | 81981 | 18: 34742637 | 81985 | 18: 34868221 | 81993 | 18: 35213610 | 81994 | 18: 35247365 |
| 81996 | 18: 35258480 | 82001 | 18: 35371180 | 82003 | 18: 35406541 | 82008 | 18: 35505165 | 82009 | 18: 35515593 |
| 82010 | 18: 35575292 | 82012 | 18: 35602009 | 82016 | 18: 35713513 | 82021 | 18: 35869059 | 82027 | 18: 35949415 |
| 82029 | 18: 35961563 | 82035 | 18: 36058481 | 82036 | 18: 36063927 | 82038 | 18: 36087372 | 82039 | 18: 36087440 |
| 82040 | 18: 36093659 | 82042 | 18: 36121381 | 82046 | 18: 36318653 | 82054 | 18: 36585013 | 82059 | 18: 36712932 |
| 82066 | 18: 36883800 | 82067 | 18: 36890174 | 82069 | 18: 36892550 | 82093 | 18: 37455502 | 82098 | 18: 37533228 |
| 82102 | 18: 37619375 | 82122 | 18: 38001454 | 82131 | 18: 38196771 | 82133 | 18: 38218416 | 82135 | 18: 38277631 |
| 82138 | 18: 38301449 | 82141 | 18: 38331320 | 82150 | 18: 38614192 | 82174 | 18: 39233646 | 82175 | 18: 39286312 |
| 82180 | 18: 39414004 | 82182 | 18: 39447432 | 82189 | 18: 39589574 | 82192 | 18: 39612914 | 82215 | 18: 40137391 |
| 82216 | 18: 40137453 | 82217 | 18: 40138746 | 82218 | 18: 40138750 | 82219 | 18: 40138985 | 82220 | 18: 40139048 |
| 82222 | 18: 40176507 | 82223 | 18: 40176673 | 82228 | 18: 40316884 | 82230 | 18: 40330132 | 82237 | 18: 40533358 |
| 82238 | 18: 40534569 | 82241 | 18: 40597479 | 82249 | 18: 40763764 | 82250 | 18: 40763815 | 82254 | 18: 40819026 |
| 82255 | 18: 40819036 | 82261 | 18: 40956148 | 82269 | 18: 41180952 | 82274 | 18: 41372533 | 82275 | 18: 41391080 |
| 82277 | 18: 41412597 | 82304 | 18: 42002651 | 82308 | 18: 42064307 | 82314 | 18: 42154655 | 82325 | 18: 42312455 |
| 82342 | 18: 42673603 | 82352 | 18: 42839761 | 82355 | 18: 42870031 | 82356 | 18: 42870306 | 82357 | 18: 42870636 |
| 82373 | 18: 43209321 | 82378 | 18: 43300251 | 82397 | 18: 43597221 | 82399 | 18: 43723768 | 82400 | 18: 43729208 |
| 82401 | 18: 43744525 | 82403 | 18: 43765343 | 82415 | 18: 44100679 | 82418 | 18: 44219776 | 82433 | 18: 44678130 |
| 82437 | 18: 44746464 | 82440 | 18: 44842043 | 82444 | 18: 44999448 | 82448 | 18: 45230454 | 82451 | 18: 45235853 |
| 82453 | 18: 45282086 | 82454 | 18: 45368501 | 82457 | 18: 45512461 | 82459 | 18: 45555708 | 82460 | 18: 45555738 |
| 82461 | 18: 45555791 | 82463 | 18: 45568910 | 82474 | 18: 45823041 | 82477 | 18: 45919734 | 82481 | 18: 46058902 |
| 82482 | 18: 46058908 | 82483 | 18: 46059262 | 82484 | 18: 46059448 | 82485 | 18: 46059625 | 82486 | 18: 46074456 |
| 82488 | 18: 46098110 | 82491 | 18: 46151119 | 82493 | 18: 46204752 | 82500 | 18: 46339447 | 82506 | 18: 46411478 |
| 82507 | 18: 46412270 | 82510 | 18: 46452569 | 82511 | 18: 46454259 | 82514 | 18: 46462113 | 82515 | 18: 46472984 |
| 82516 | 18: 46474223 | 82517 | 18: 46474231 | 82518 | 18: 46474239 | 82519 | 18: 46474366 | 82520 | 18: 46480475 |
| 82521 | 18: 46481003 | 82522 | 18: 46481046 | 82523 | 18: 46481053 | 82524 | 18: 46481480 | 82525 | 18: 46481499 |
| 82526 | 18: 46489662 | 82527 | 18: 46490303 | 82528 | 18: 46490881 | 82529 | 18: 46498795 | 82530 | 18: 46499716 |
| 82531 | 18: 46500155 | 82532 | 18: 46511353 | 82533 | 18: 46512103 | 82534 | 18: 46512104 | 82535 | 18: 46523855 |
| 82536 | 18: 46528787 | 82540 | 18: 46627346 | 82542 | 18: 46696595 | 82543 | 18: 46697798 | 82544 | 18: 46727043 |
| 82546 | 18: 46727075 | 82547 | 18: 46731490 | 82548 | 18: 46731757 | 82549 | 18: 46731785 | 82550 | 18: 46731861 |
| 82551 | 18: 46742887 | 82552 | 18: 46742932 | 82553 | 18: 46742940 | 82554 | 18: 46743003 | 82555 | 18: 46745292 |
| 82556 | 18: 46745318 | 82557 | 18: 46745397 | 82558 | 18: 46745402 | 82559 | 18: 46747438 | 82560 | 18: 46747523 |
| 82561 | 18: 46751198 | 82562 | 18: 46751982 | 82563 | 18: 46751987 | 82564 | 18: 46752090 | 82565 | 18: 46754516 |
| 82567 | 18: 46762245 | 82568 | 18: 46762248 | 82569 | 18: 46763772 | 82570 | 18: 46764309 | 82571 | 18: 46764530 |
| 82572 | 18: 46764820 | 82573 | 18: 46764937 | 82574 | 18: 46765974 | 82575 | 18: 46766007 | 82576 | 18: 46766010 |
| 82577 | 18: 46771417 | 82578 | 18: 46772451 | 82581 | 18: 46793532 | 82585 | 18: 46926291 | 82586 | 18: 46926325 |
| 82587 | 18: 46935389 | 82588 | 18: 46936632 | 82589 | 18: 46936776 | 82592 | 18: 46961882 | 82593 | 18: 46987640 |
| 82606 | 18: 47331147 | 82607 | 18: 47350791 | 82609 | 18: 47380300 | 82614 | 18: 47561882 | 82615 | 18: 47569792 |
| 82616 | 18: 47584273 | 82623 | 18: 47724055 | 82630 | 18: 47880052 | 82632 | 18: 47904454 | 82633 | 18: 47905402 |
| 82635 | 18: 47914575 | 82638 | 18: 47982264 | 82639 | 18: 47986634 | 82645 | 18: 48080577 | 82646 | 18: 48082036 |
| 82647 | 18: 48092307 | 82648 | 18: 48093267 | 82649 | 18: 48094554 | 82650 | 18: 48095808 | 82651 | 18: 48097168 |
| 82652 | 18: 48098873 | 82653 | 18: 48099751 | 82654 | 18: 48100359 | 82655 | 18: 48102403 | 82656 | 18: 48103383 |
| 82657 | 18: 48105090 | 82658 | 18: 48105806 | 82660 | 18: 48115016 | 82661 | 18: 48119818 | 82662 | 18: 48164016 |
| 82669 | 18: 48328458 | 82674 | 18: 48486626 | 82677 | 18: 48540681 | 82678 | 18: 48540711 | 82681 | 18: 48550284 |
| 82682 | 18: 48552506 | 82684 | 18: 48648276 | 82685 | 18: 48661989 | 82692 | 18: 48824317 | 82700 | 18: 49125312 |
| 82709 | 18: 49431844 | 82710 | 18: 49440743 | 82718 | 18: 49685722 | 82720 | 18: 49690710 | 82722 | 18: 49774209 |
| 82727 | 18: 49905675 | 82734 | 18: 49974216 | 82744 | 18: 50307933 | 82768 | 18: 50909576 | 82776 | 18: 51192874 |
| 82780 | 18: 51273762 | 82784 | 18: 51393314 | 82799 | 18: 51647623 | 82801 | 18: 51684659 | 82803 | 18: 51687197 |
| 82804 | 18: 51691345 | 82805 | 18: 51691402 | 82806 | 18: 51691426 | 82807 | 18: 51691907 | 82808 | 18: 51692088 |
| 82810 | 18: 51703006 | 82811 | 18: 51703434 | 82813 | 18: 51710422 | 82820 | 18: 51868158 | 82821 | 18: 51868247 |
| 82822 | 18: 51877833 | 82823 | 18: 51888735 | 82826 | 18: 51959716 | 82833 | 18: 52170747 | 82843 | 18: 52408540 |
| 82844 | 18: 52434788 | 82846 | 18: 52463921 | 82848 | 18: 52526615 | 82849 | 18: 52569137 | 82850 | 18: 52586631 |
| 82856 | 18: 52750690 | 82863 | 18: 53056273 | 82864 | 18: 53066684 | 82876 | 18: 53447947 | 82877 | 18: 53462716 |
| 82878 | 18: 53515653 | 82880 | 18: 53545259 | 82883 | 18: 53565634 | 82885 | 18: 53596284 | 82889 | 18: 53631016 |
| 82890 | 18: 53635483 | 82891 | 18: 53666625 | 82892 | 18: 53680050 | 82902 | 18: 53873089 | 82903 | 18: 53882150 |
| 82907 | 18: 53924797 | 82908 | 18: 53926298 | 82909 | 18: 53938427 | 82910 | 18: 53965820 | 82911 | 18: 53970987 |
| 82912 | 18: 53987026 | 82913 | 18: 53988178 | 82917 | 18: 54051234 | 82922 | 18: 54129853 | 82924 | 18: 54256059 |
| 82929 | 18: 54326329 | 82930 | 18: 54348038 | 82931 | 18: 54348158 | 82932 | 18: 54356059 | 82943 | 18: 54594314 |
| 82945 | 18: 54606774 | 82946 | 18: 54607245 | 82947 | 18: 54615284 | 82948 | 18: 54615550 | 82949 | 18: 54618878 |
| 82951 | 18: 54632037 | 82952 | 18: 54674333 | 82956 | 18: 54725999 | 82957 | 18: 54726122 | 82958 | 18: 54732855 |
| 82965 | 18: 54920625 | 82978 | 18: 55105119 | 82980 | 18: 55125421 | 82981 | 18: 55130089 | 82991 | 18: 55397342 |
| 82993 | 18: 55448102 | 82994 | 18: 55479044 | 83003 | 18: 55722088 | 83006 | 18: 55855289 | 83010 | 18: 55908909 |
| 83022 | 18: 56235646 | 83025 | 18: 56256914 | 83027 | 18: 56286701 | 83028 | 18: 56286760 | 83030 | 18: 56311848 |
| 83037 | 18: 56458961 | 83039 | 18: 56480496 | 83040 | 18: 56481635 | 83041 | 18: 56500189 | 83043 | 18: 56516815 |
| 83046 | 18: 56550331 | 83051 | 18: 56657542 | 83053 | 18: 56669005 | 83054 | 18: 56669128 | 83055 | 18: 56679475 |
| 83114 | 18: 57068537 | 83115 | 18: 57072585 | 83119 | 18: 57151314 | 83120 | 18: 57195331 | 83126 | 18: 57435685 |
| 83134 | 18: 57498462 | 83140 | 18: 57652070 | 83141 | 18: 57658393 | 83142 | 18: 57662151 | 83143 | 18: 57673429 |
| 83149 | 18: 57750585 | 83150 | 18: 57764229 | 83151 | 18: 57774781 | 83159 | 18: 57875057 | 83160 | 18: 57880006 |
| 83162 | 18: 57922196 | 83163 | 18: 57922208 | 83168 | 18: 57950000 | 83184 | 18: 58213698 | 83185 | 18: 58215105 |
| 83186 | 18: 58221738 | 83187 | 18: 58237470 | 83189 | 18: 58270823 | 83190 | 18: 58280185 | 83191 | 18: 58285064 |
| 83192 | 18: 58286528 | 83193 | 18: 58288374 | 83194 | 18: 58289386 | 83195 | 18: 58290105 | 83196 | 18: 58292173 |
| 83197 | 18: 58292702 | 83198 | 18: 58293461 | 83199 | 18: 58294515 | 83200 | 18: 58296803 | 83201 | 18: 58297833 |
| 83202 | 18: 58298676 | 83203 | 18: 58302578 | 83204 | 18: 58303580 | 83205 | 18: 58304589 | 83206 | 18: 58306121 |
| 83207 | 18: 58308817 | 83208 | 18: 58309408 | 83209 | 18: 58311748 | 83210 | 18: 58314758 | 83211 | 18: 58319283 |
| 83213 | 18: 58328404 | 83214 | 18: 58335688 | 83215 | 18: 58336548 | 83217 | 18: 58339398 | 83218 | 18: 58340297 |
| 83219 | 18: 58341452 | 83220 | 18: 58343184 | 83221 | 18: 58346217 | 83222 | 18: 58359203 | 83223 | 18: 58360688 |
| 83224 | 18: 58362462 | 83225 | 18: 58369410 | 83226 | 18: 58372976 | 83228 | 18: 58501449 | 83259 | 18: 59941034 |
| 83264 | 18: 60057741 | 83276 | 18: 60557876 | 83284 | 18: 60892284 | 83285 | 18: 60892325 | 83286 | 18: 60892345 |
| 83287 | 18: 60908498 | 83296 | 18: 61230100 | 83297 | 18: 61248798 | 83306 | 18: 61528542 | 83314 | 18: 61669548 |
| 83316 | 18: 61688550 | 83327 | 18: 61924215 | 83328 | 18: 61925438 | 83329 | 18: 61925595 | 83331 | 18: 61938092 |
| 83332 | 18: 61941006 | 83333 | 18: 61948334 | 83334 | 18: 61949940 | 83336 | 18: 61962954 | 83337 | 18: 61968568 |
| 83338 | 18: 61969417 | 83339 | 18: 61975282 | 83340 | 18: 61976235 | 83341 | 18: 61977792 | 83343 | 18: 61979272 |
| 83344 | 18: 61986664 | 83345 | 18: 61987641 | 83347 | 18: 62017097 | 83348 | 18: 62031220 | 83350 | 18: 62050366 |
| 83351 | 18: 62055500 | 83352 | 18: 62069703 | 83355 | 18: 62098858 | 83356 | 18: 62119696 | 83365 | 18: 62301361 |
| 83369 | 18: 62396313 | 83370 | 18: 62411556 | 83371 | 18: 62412333 | 83372 | 18: 62491921 | 83391 | 18: 63189938 |
| 83392 | 18: 63193118 | 83393 | 18: 63203763 | 83396 | 18: 63217724 | 83397 | 18: 63217760 | 83398 | 18: 63217761 |
| 83401 | 18: 63282873 | 83406 | 18: 63442841 | 83407 | 18: 63443616 | 83410 | 18: 63449366 | 83413 | 18: 63478461 |
| 83420 | 18: 63566906 | 83435 | 18: 63851165 | 83438 | 18: 63870217 | 83439 | 18: 63870560 | 83440 | 18: 63870634 |
| 83441 | 18: 63878287 | 83446 | 18: 64020285 | 83448 | 18: 64103695 | 83449 | 18: 64107247 | 83450 | 18: 64118087 |
| 83459 | 18: 64261304 | 83464 | 18: 64351790 | 83473 | 18: 64453963 | 83475 | 18: 64457710 | 83480 | 18: 64586875 |
| 83481 | 18: 64586957 | 83483 | 18: 64587029 | 83484 | 18: 64588207 | 83485 | 18: 64588277 | 83486 | 18: 64594228 |
| 83489 | 18: 64607295 | 83493 | 18: 64684413 | 83500 | 18: 64871544 | 83506 | 18: 64979546 | 83507 | 18: 64989257 |
| 83508 | 18: 65000708 | 83512 | 18: 65024355 | 83513 | 18: 65052564 | 83514 | 18: 65058054 | 83515 | 18: 65058108 |
| 83516 | 18: 65058135 | 83522 | 18: 65143297 | 83538 | 18: 65213339 | 83546 | 18: 65269401 | 83570 | 18: 65467804 |
| 83573 | 18: 65499590 | 83576 | 18: 65531536 | 83582 | 18: 65680076 | 83584 | 18: 65778621 | 83611 | 19: 1044735 |
| 83641 | 19: 2028992 | 83643 | 19: 2087607 | 83647 | 19: 2189142 | 83648 | 19: 2189181 | 83669 | 19: 3019557 |
| 83676 | 19: 3224135 | 83684 | 19: 3507467 | 83688 | 19: 3693922 | 83689 | 19: 3693998 | 83690 | 19: 3694048 |
| 83691 | 19: 3717004 | 83712 | 19: 4248938 | 83719 | 19: 4532769 | 83732 | 19: 4885934 | 83737 | 19: 5057366 |
| 83738 | 19: 5077598 | 83742 | 19: 5131889 | 83743 | 19: 5144117 | 83748 | 19: 5302256 | 83758 | 19: 5544452 |
| 83759 | 19: 5550410 | 83770 | 19: 5841378 | 83773 | 19: 5911200 | 83777 | 19: 6008981 | 83779 | 19: 6069254 |
| 83780 | 19: 6080767 | 83802 | 19: 6741959 | 83803 | 19: 6742031 | 83804 | 19: 6744112 | 83808 | 19: 6821054 |
| 83810 | 19: 6854445 | 83823 | 19: 7084364 | 83825 | 19: 7139360 | 83830 | 19: 7250907 | 83835 | 19: 7311199 |
| 83857 | 19: 7610709 | 83860 | 19: 7655487 | 83867 | 19: 7877242 | 83870 | 19: 7955627 | 83874 | 19: 8012167 |
| 83882 | 19: 8102634 | 83884 | 19: 8134333 | 83888 | 19: 8242768 | 83890 | 19: 8251385 | 83891 | 19: 8253685 |
| 83892 | 19: 8253738 | 83893 | 19: 8256106 | 83894 | 19: 8257817 | 83896 | 19: 8303146 | 83897 | 19: 8307007 |
| 83899 | 19: 8318345 | 83909 | 19: 8375150 | 83914 | 19: 8447018 | 83917 | 19: 8481149 | 83919 | 19: 8538010 |
| 83928 | 19: 8786239 | 83937 | 19: 8921388 | 83938 | 19: 8940266 | 83939 | 19: 8940272 | 83946 | 19: 9148135 |
| 83948 | 19: 9161538 | 83951 | 19: 9186570 | 83952 | 19: 9186624 | 83953 | 19: 9186684 | 83954 | 19: 9186696 |
| 83955 | 19: 9186752 | 83960 | 19: 9321464 | 83963 | 19: 9325083 | 83964 | 19: 9329176 | 83971 | 19: 9494606 |
| 83973 | 19: 9527082 | 83974 | 19: 9549010 | 83976 | 19: 9584099 | 83978 | 19: 9638098 | 83996 | 19: 10144335 |
| 84000 | 19: 10161366 | 84001 | 19: 10185318 | 84002 | 19: 10193665 | 84004 | 19: 10203916 | 84005 | 19: 10212410 |
| 84006 | 19: 10222141 | 84012 | 19: 10311673 | 84015 | 19: 10357642 | 84018 | 19: 10410339 | 84027 | 19: 10608070 |
| 84028 | 19: 10663875 | 84033 | 19: 10816146 | 84048 | 19: 11195343 | 84049 | 19: 11206085 | 84050 | 19: 11223181 |
| 84051 | 19: 11223607 | 84074 | 19: 11794721 | 84081 | 19: 12080713 | 84098 | 19: 12520276 | 84106 | 19: 12624098 |
| 84115 | 19: 12851003 | 84118 | 19: 12942065 | 84119 | 19: 12942066 | 84138 | 19: 13385076 | 84153 | 19: 13539153 |
| 84154 | 19: 13540416 | 84156 | 19: 13546434 | 84157 | 19: 13553916 | 84158 | 19: 13554659 | 84160 | 19: 13567678 |
| 84161 | 19: 13567681 | 84162 | 19: 13569519 | 84163 | 19: 13589753 | 84167 | 19: 13627880 | 84168 | 19: 13629329 |
| 84182 | 19: 13922585 | 84184 | 19: 13992338 | 84185 | 19: 13992353 | 84193 | 19: 14098428 | 84197 | 19: 14234372 |
| 84199 | 19: 14285305 | 84202 | 19: 14359808 | 84206 | 19: 14455998 | 84207 | 19: 14458140 | 84208 | 19: 14460416 |
| 84209 | 19: 14461216 | 84212 | 19: 14524347 | 84215 | 19: 14594291 | 84231 | 19: 14987147 | 84233 | 19: 14997330 |
| 84236 | 19: 15044294 | 84238 | 19: 15079297 | 84265 | 19: 15653982 | 84275 | 19: 15950588 | 84282 | 19: 16153652 |
| 84285 | 19: 16179422 | 84286 | 19: 16179458 | 84290 | 19: 16243199 | 84291 | 19: 16245110 | 84294 | 19: 16316143 |
| 84296 | 19: 16414613 | 84297 | 19: 16414653 | 84298 | 19: 16414656 | 84299 | 19: 16414688 | 84300 | 19: 16414750 |
| 84301 | 19: 16414769 | 84305 | 19: 16538181 | 84306 | 19: 16538222 | 84307 | 19: 16538228 | 84308 | 19: 16538286 |
| 84309 | 19: 16538332 | 84314 | 19: 16680352 | 84315 | 19: 16698614 | 84316 | 19: 16701167 | 84317 | 19: 16704714 |
| 84318 | 19: 16710947 | 84319 | 19: 16712329 | 84320 | 19: 16715359 | 84329 | 19: 16900771 | 84344 | 19: 17139083 |
| 84345 | 19: 17193285 | 84347 | 19: 17219724 | 84354 | 19: 17346967 | 84358 | 19: 17413575 | 84360 | 19: 17439500 |
| 84361 | 19: 17463604 | 84362 | 19: 17463752 | 84363 | 19: 17463766 | 84364 | 19: 17463785 | 84366 | 19: 17476802 |
| 84367 | 19: 17485925 | 84381 | 19: 17839100 | 84382 | 19: 17840344 | 84383 | 19: 17847133 | 84388 | 19: 17935227 |
| 84390 | 19: 18002956 | 84391 | 19: 18012136 | 84405 | 19: 18364670 | 84427 | 19: 18812123 | 84429 | 19: 18935333 |
| 84434 | 19: 19027484 | 84438 | 19: 19066522 | 84440 | 19: 19164165 | 84441 | 19: 19167861 | 84443 | 19: 19248544 |
| 84448 | 19: 19342984 | 84455 | 19: 19497354 | 84456 | 19: 19503339 | 84469 | 19: 19832430 | 84470 | 19: 19832910 |
| 84473 | 19: 19891932 | 84478 | 19: 20010169 | 84479 | 19: 20010699 | 84480 | 19: 20021077 | 84481 | 19: 20024935 |
| 84482 | 19: 20039821 | 84483 | 19: 20039838 | 84484 | 19: 20049560 | 84486 | 19: 20064375 | 84487 | 19: 20065239 |
| 84488 | 19: 20065746 | 84489 | 19: 20067184 | 84490 | 19: 20067197 | 84496 | 19: 20169459 | 84497 | 19: 20252737 |
| 84498 | 19: 20269891 | 84499 | 19: 20272304 | 84500 | 19: 20272993 | 84501 | 19: 20272997 | 84502 | 19: 20287832 |
| 84503 | 19: 20347363 | 84504 | 19: 20347364 | 84505 | 19: 20347398 | 84506 | 19: 20347408 | 84507 | 19: 20347436 |
| 84508 | 19: 20347583 | 84511 | 19: 20397604 | 84513 | 19: 20404265 | 84514 | 19: 20406390 | 84515 | 19: 20407245 |
| 84516 | 19: 20407776 | 84517 | 19: 20407895 | 84518 | 19: 20408583 | 84519 | 19: 20409859 | 84520 | 19: 20410892 |
| 84521 | 19: 20411434 | 84522 | 19: 20412101 | 84523 | 19: 20413435 | 84524 | 19: 20414800 | 84525 | 19: 20415545 |
| 84526 | 19: 20415733 | 84527 | 19: 20416065 | 84528 | 19: 20416081 | 84529 | 19: 20416676 | 84530 | 19: 20417918 |
| 84531 | 19: 20418417 | 84532 | 19: 20418508 | 84533 | 19: 20418613 | 84535 | 19: 20421499 | 84536 | 19: 20423129 |
| 84537 | 19: 20427231 | 84538 | 19: 20443801 | 84539 | 19: 20448136 | 84540 | 19: 20449515 | 84541 | 19: 20450452 |
| 84542 | 19: 20451130 | 84543 | 19: 20454459 | 84544 | 19: 20459782 | 84545 | 19: 20464970 | 84546 | 19: 20466789 |
| 84547 | 19: 20468157 | 84548 | 19: 20469610 | 84557 | 19: 20885539 | 84561 | 19: 20948913 | 84570 | 19: 21194708 |
| 84571 | 19: 21195341 | 84580 | 19: 21541775 | 84582 | 19: 21606050 | 84583 | 19: 21606066 | 84584 | 19: 21606170 |
| 84586 | 19: 21607755 | 84587 | 19: 21607870 | 84588 | 19: 21657448 | 84596 | 19: 21761419 | 84601 | 19: 21810539 |
| 84602 | 19: 21810883 | 84608 | 19: 22007200 | 84613 | 19: 22125908 | 84617 | 19: 22209844 | 84618 | 19: 22216194 |
| 84619 | 19: 22216936 | 84620 | 19: 22218946 | 84621 | 19: 22223761 | 84622 | 19: 22225302 | 84624 | 19: 22228117 |
| 84625 | 19: 22228881 | 84626 | 19: 22238525 | 84629 | 19: 22288784 | 84632 | 19: 22387067 | 84637 | 19: 22435985 |
| 84640 | 19: 22580315 | 84642 | 19: 22630921 | 84645 | 19: 22686577 | 84649 | 19: 22777911 | 84653 | 19: 22919648 |
| 84654 | 19: 22933829 | 84655 | 19: 22937788 | 84659 | 19: 23047864 | 84663 | 19: 23089782 | 84665 | 19: 23103859 |
| 84666 | 19: 23125220 | 84677 | 19: 23322846 | 84678 | 19: 23328857 | 84691 | 19: 23748456 | 84692 | 19: 23762942 |
| 84696 | 19: 23795890 | 84699 | 19: 23844740 | 84709 | 19: 24085976 | 84710 | 19: 24086312 | 84711 | 19: 24086389 |
| 84712 | 19: 24100573 | 84713 | 19: 24119941 | 84714 | 19: 24120015 | 84715 | 19: 24120089 | 84716 | 19: 24120654 |
| 84717 | 19: 24120726 | 84719 | 19: 24158070 | 84720 | 19: 24171676 | 84721 | 19: 24190844 | 84722 | 19: 24190846 |
| 84723 | 19: 24190851 | 84724 | 19: 24190893 | 84725 | 19: 24201030 | 84726 | 19: 24201096 | 84727 | 19: 24203359 |
| 84728 | 19: 24203402 | 84729 | 19: 24203454 | 84730 | 19: 24204657 | 84731 | 19: 24206255 | 84732 | 19: 24206362 |
| 84733 | 19: 24210049 | 84734 | 19: 24211193 | 84740 | 19: 24352581 | 84749 | 19: 24484569 | 84752 | 19: 24575685 |
| 84757 | 19: 24628286 | 84758 | 19: 24643342 | 84760 | 19: 24654902 | 84763 | 19: 24710033 | 84766 | 19: 24729440 |
| 84767 | 19: 24730057 | 84768 | 19: 24732020 | 84769 | 19: 24736477 | 84770 | 19: 24737388 | 84771 | 19: 24739849 |
| 84772 | 19: 24743924 | 84773 | 19: 24747961 | 84774 | 19: 24753662 | 84776 | 19: 24761701 | 84777 | 19: 24791465 |
| 84778 | 19: 24832457 | 84779 | 19: 24834421 | 84780 | 19: 24843420 | 84782 | 19: 24848682 | 84783 | 19: 24859742 |
| 84784 | 19: 24862477 | 84787 | 19: 24938845 | 84788 | 19: 24958116 | 84789 | 19: 24958789 | 84793 | 19: 25015319 |
| 84795 | 19: 25042003 | 84797 | 19: 25049455 | 84799 | 19: 25055788 | 84802 | 19: 25112769 | 84808 | 19: 25148679 |
| 84809 | 19: 25149070 | 84810 | 19: 25167024 | 84815 | 19: 25292038 | 84843 | 19: 25636984 | 84847 | 19: 25714307 |
| 84866 | 19: 26042854 | 84867 | 19: 26047036 | 84868 | 19: 26047065 | 84869 | 19: 26047497 | 84870 | 19: 26047588 |
| 84871 | 19: 26048186 | 84872 | 19: 26048187 | 84873 | 19: 26048200 | 84874 | 19: 26048237 | 84875 | 19: 26048249 |
| 84876 | 19: 26050069 | 84877 | 19: 26050076 | 84878 | 19: 26050122 | 84894 | 19: 26326260 | 84895 | 19: 26328504 |
| 84896 | 19: 26330382 | 84897 | 19: 26362443 | 84903 | 19: 26386801 | 84909 | 19: 26431555 | 84911 | 19: 26433035 |
| 84913 | 19: 26435773 | 84914 | 19: 26437827 | 84915 | 19: 26438388 | 84919 | 19: 26443714 | 84920 | 19: 26452130 |
| 84921 | 19: 26455955 | 84923 | 19: 26459081 | 84924 | 19: 26461611 | 84929 | 19: 26467372 | 84933 | 19: 26494500 |
| 84934 | 19: 26498170 | 84937 | 19: 26508308 | 84938 | 19: 26509449 | 84940 | 19: 26512520 | 84943 | 19: 26571898 |
| 84967 | 19: 26833517 | 84968 | 19: 26834363 | 84971 | 19: 26944800 | 84972 | 19: 26944904 | 84991 | 19: 27851735 |
| 84994 | 19: 27919465 | 84995 | 19: 27929802 | 84997 | 19: 27974645 | 84999 | 19: 27986943 | 85003 | 19: 28063061 |
| 85022 | 19: 28341985 | 85027 | 19: 28441795 | 85033 | 19: 28599438 | 85039 | 19: 28789212 | 85055 | 19: 29142555 |
| 85056 | 19: 29146537 | 85057 | 19: 29165645 | 85060 | 19: 29185159 | 85062 | 19: 29191189 | 85070 | 19: 29360624 |
| 85071 | 19: 29360635 | 85077 | 19: 29516407 | 85082 | 19: 29601656 | 85088 | 19: 29667582 | 85089 | 19: 29671837 |
| 85093 | 19: 29784730 | 85094 | 19: 29784751 | 85095 | 19: 29784815 | 85106 | 19: 30101489 | 85109 | 19: 30199565 |
| 85125 | 19: 30493276 | 85143 | 19: 30892000 | 85145 | 19: 30952848 | 85148 | 19: 31087410 | 85150 | 19: 31146132 |
| 85153 | 19: 31161313 | 85154 | 19: 31165405 | 85155 | 19: 31165486 | 85160 | 19: 31346087 | 85161 | 19: 31369446 |
| 85193 | 19: 32163965 | 85194 | 19: 32172115 | 85201 | 19: 32267525 | 85223 | 19: 32751602 | 85224 | 19: 32768693 |
| 85225 | 19: 32800795 | 85226 | 19: 32804878 | 85233 | 19: 33007630 | 85234 | 19: 33019809 | 85238 | 19: 33125153 |
| 85243 | 19: 33187115 | 85246 | 19: 33215711 | 85249 | 19: 33262257 | 85255 | 19: 33294100 | 85267 | 19: 33578331 |
| 85268 | 19: 33594931 | 85269 | 19: 33596340 | 85271 | 19: 33623310 | 85279 | 19: 33954621 | 85281 | 19: 33998092 |
| 85282 | 19: 34005543 | 85283 | 19: 34005879 | 85286 | 19: 34041049 | 85303 | 19: 34565878 | 85306 | 19: 34576319 |
| 85313 | 19: 34769105 | 85314 | 19: 34779947 | 85315 | 19: 34782489 | 85316 | 19: 34786152 | 85317 | 19: 34795515 |
| 85318 | 19: 34801199 | 85319 | 19: 34814559 | 85320 | 19: 34815409 | 85321 | 19: 34821534 | 85322 | 19: 34822575 |
| 85323 | 19: 34823488 | 85324 | 19: 34824385 | 85325 | 19: 34825185 | 85326 | 19: 34826088 | 85327 | 19: 34826902 |
| 85328 | 19: 34827822 | 85329 | 19: 34832510 | 85330 | 19: 34833809 | 85331 | 19: 34835066 | 85332 | 19: 34839003 |
| 85333 | 19: 34839771 | 85334 | 19: 34840668 | 85335 | 19: 34842069 | 85336 | 19: 34843150 | 85337 | 19: 34850412 |
| 85338 | 19: 34851901 | 85339 | 19: 34860953 | 85340 | 19: 34867998 | 85341 | 19: 34873374 | 85342 | 19: 34876608 |
| 85343 | 19: 34877505 | 85344 | 19: 34884414 | 85346 | 19: 34893695 | 85348 | 19: 34911757 | 85349 | 19: 34917689 |
| 85350 | 19: 34924937 | 85351 | 19: 34940577 | 85352 | 19: 34941647 | 85353 | 19: 34942605 | 85360 | 19: 35015390 |
| 85385 | 19: 35630509 | 85391 | 19: 35778297 | 85404 | 19: 36050548 | 85405 | 19: 36052759 | 85408 | 19: 36139902 |
| 85410 | 19: 36161222 | 85412 | 19: 36183218 | 85413 | 19: 36186920 | 85414 | 19: 36204759 | 85415 | 19: 36205924 |
| 85417 | 19: 36230644 | 85418 | 19: 36232203 | 85419 | 19: 36233310 | 85420 | 19: 36234144 | 85421 | 19: 36235392 |
| 85422 | 19: 36236022 | 85423 | 19: 36236961 | 85424 | 19: 36238112 | 85425 | 19: 36239325 | 85426 | 19: 36242008 |
| 85430 | 19: 36285683 | 85432 | 19: 36304660 | 85436 | 19: 36349079 | 85443 | 19: 36515692 | 85444 | 19: 36516381 |
| 85445 | 19: 36540830 | 85446 | 19: 36572979 | 85447 | 19: 36574100 | 85448 | 19: 36574859 | 85449 | 19: 36574986 |
| 85450 | 19: 36576060 | 85451 | 19: 36577180 | 85452 | 19: 36577974 | 85453 | 19: 36585052 | 85454 | 19: 36585096 |
| 85455 | 19: 36585153 | 85456 | 19: 36586555 | 85457 | 19: 36588245 | 85458 | 19: 36598037 | 85460 | 19: 36659503 |
| 85478 | 19: 37057391 | 85480 | 19: 37085911 | 85503 | 19: 37768695 | 85512 | 19: 38027080 | 85513 | 19: 38112442 |
| 85514 | 19: 38115441 | 85515 | 19: 38135659 | 85519 | 19: 38330215 | 85523 | 19: 38378249 | 85535 | 19: 38818642 |
| 85537 | 19: 38825404 | 85543 | 19: 39018067 | 85546 | 19: 39173581 | 85548 | 19: 39195126 | 85554 | 19: 39319605 |
| 85555 | 19: 39319939 | 85556 | 19: 39324677 | 85557 | 19: 39373787 | 85558 | 19: 39403214 | 85560 | 19: 39423726 |
| 85564 | 19: 39558542 | 85568 | 19: 39592912 | 85572 | 19: 39664178 | 85574 | 19: 39702056 | 85575 | 19: 39761996 |
| 85585 | 19: 40136281 | 85589 | 19: 40231461 | 85591 | 19: 40243945 | 85592 | 19: 40244440 | 85593 | 19: 40244746 |
| 85594 | 19: 40250106 | 85603 | 19: 40340447 | 85608 | 19: 40504497 | 85611 | 19: 40571544 | 85617 | 19: 40687629 |
| 85634 | 19: 40969791 | 85636 | 19: 40998269 | 85637 | 19: 41009813 | 85639 | 19: 41075395 | 85641 | 19: 41125615 |
| 85642 | 19: 41125654 | 85643 | 19: 41126901 | 85644 | 19: 41134261 | 85645 | 19: 41134335 | 85646 | 19: 41167136 |
| 85666 | 19: 41672120 | 85671 | 19: 41759325 | 85681 | 19: 41844566 | 85683 | 19: 41866585 | 85685 | 19: 41908524 |
| 85687 | 19: 41944555 | 85689 | 19: 41954155 | 85694 | 19: 42058566 | 85695 | 19: 42063521 | 85698 | 19: 42131015 |
| 85701 | 19: 42144465 | 85704 | 19: 42227260 | 85710 | 19: 42333202 | 85712 | 19: 42345437 | 85719 | 19: 42426465 |
| 85728 | 19: 42644624 | 85731 | 19: 42719942 | 85732 | 19: 42726195 | 85736 | 19: 42848843 | 85738 | 19: 42887598 |
| 85741 | 19: 43035501 | 85742 | 19: 43043947 | 85743 | 19: 43049014 | 85750 | 19: 43333514 | 85759 | 19: 43422824 |
| 85768 | 19: 43443947 | 85775 | 19: 43599080 | 85777 | 19: 43713425 | 85783 | 19: 43816258 | 85784 | 19: 43830215 |
| 85787 | 19: 43926648 | 85790 | 19: 43991252 | 85793 | 19: 44072509 | 85795 | 19: 44116524 | 85797 | 19: 44122310 |
| 85799 | 19: 44173520 | 85800 | 19: 44177664 | 85801 | 19: 44178974 | 85806 | 19: 44309222 | 85808 | 19: 44359879 |
| 85809 | 19: 44385146 | 85813 | 19: 44477082 | 85818 | 19: 44577139 | 85821 | 19: 44615861 | 85822 | 19: 44651270 |
| 85823 | 19: 44673344 | 85824 | 19: 44673867 | 85826 | 19: 44715813 | 85838 | 19: 45013727 | 85841 | 19: 45073651 |
| 85861 | 19: 45630233 | 85892 | 19: 45816366 | 85893 | 19: 45817041 | 85896 | 19: 45844326 | 85898 | 19: 45920605 |
| 85899 | 19: 45934999 | 85900 | 19: 45949710 | 85909 | 19: 46106226 | 85918 | 19: 46396057 | 85928 | 19: 46762829 |
| 85933 | 19: 46867389 | 85944 | 19: 47138439 | 85945 | 19: 47138441 | 85946 | 19: 47138654 | 85947 | 19: 47138655 |
| 85948 | 19: 47138674 | 85949 | 19: 47138684 | 85950 | 19: 47139281 | 85958 | 19: 47384175 | 85959 | 19: 47402508 |
| 85960 | 19: 47405017 | 85974 | 19: 47676904 | 85975 | 19: 47735369 | 85979 | 19: 47763256 | 85984 | 19: 47930272 |
| 85986 | 19: 47955876 | 85987 | 19: 47966555 | 85990 | 19: 48025233 | 85993 | 19: 48071339 | 85994 | 19: 48075239 |
| 85996 | 19: 48084284 | 86000 | 19: 48118256 | 86001 | 19: 48142220 | 86005 | 19: 48258274 | 86006 | 19: 48266963 |
| 86012 | 19: 48389397 | 86016 | 19: 48418566 | 86022 | 19: 48563047 | 86028 | 19: 48709127 | 86032 | 19: 48880795 |
| 86042 | 19: 49040357 | 86051 | 19: 49233318 | 86055 | 19: 49277856 | 86063 | 19: 49478759 | 86064 | 19: 49483816 |
| 86065 | 19: 49494727 | 86068 | 19: 49575523 | 86076 | 19: 49772893 | 86078 | 19: 49810028 | 86085 | 19: 49891419 |
| 86092 | 19: 50063999 | 86094 | 19: 50095971 | 86095 | 19: 50107587 | 86096 | 19: 50123299 | 86099 | 19: 50167379 |
| 86100 | 19: 50172634 | 86101 | 19: 50173024 | 86102 | 19: 50173036 | 86103 | 19: 50173360 | 86104 | 19: 50173420 |
| 86116 | 19: 50607587 | 86118 | 19: 50662334 | 86122 | 19: 50682193 | 86139 | 19: 50783155 | 86140 | 19: 50784393 |
| 86142 | 19: 50788319 | 86144 | 19: 50790149 | 86145 | 19: 50790973 | 86146 | 19: 50791045 | 86147 | 19: 50817214 |
| 86148 | 19: 50817251 | 86150 | 19: 50838297 | 86151 | 19: 50838402 | 86152 | 19: 50855700 | 86153 | 19: 50856195 |
| 86154 | 19: 50883463 | 86155 | 19: 50890139 | 86156 | 19: 50892965 | 86157 | 19: 50893275 | 86158 | 19: 50893330 |
| 86159 | 19: 50893412 | 86160 | 19: 50894507 | 86164 | 19: 51146306 | 86165 | 19: 51148044 | 86166 | 19: 51150289 |
| 86167 | 19: 51151634 | 86168 | 19: 51152336 | 86169 | 19: 51152846 | 86170 | 19: 51153433 | 86171 | 19: 51155662 |
| 86172 | 19: 51156301 | 86173 | 19: 51158110 | 86174 | 19: 51172862 | 86175 | 19: 51173451 | 86176 | 19: 51179705 |
| 86177 | 19: 51181597 | 86178 | 19: 51184543 | 86179 | 19: 51185390 | 86180 | 19: 51186411 | 86181 | 19: 51186781 |
| 86182 | 19: 51186933 | 86183 | 19: 51187678 | 86184 | 19: 51192006 | 86185 | 19: 51194044 | 86186 | 19: 51195370 |
| 86187 | 19: 51196420 | 86188 | 19: 51197290 | 86189 | 19: 51197844 | 86190 | 19: 51198485 | 86191 | 19: 51200087 |
| 86192 | 19: 51202775 | 86193 | 19: 51203906 | 86194 | 19: 51204912 | 86195 | 19: 51206147 | 86196 | 19: 51206982 |
| 86197 | 19: 51208278 | 86198 | 19: 51209192 | 86199 | 19: 51215165 | 86213 | 19: 51584353 | 86214 | 19: 51614996 |
| 86215 | 19: 51624830 | 86219 | 19: 51657080 | 86220 | 19: 51674934 | 86221 | 19: 51674957 | 86222 | 19: 51674972 |
| 86223 | 19: 51676635 | 86225 | 19: 51688593 | 86226 | 19: 51689866 | 86238 | 19: 52025573 | 86243 | 19: 52232662 |
| 86246 | 19: 52242008 | 86261 | 19: 52716129 | 86275 | 19: 52957714 | 86277 | 19: 53006255 | 86279 | 19: 53041756 |
| 86281 | 19: 53068039 | 86285 | 19: 53125990 | 86286 | 19: 53132946 | 86301 | 19: 53433970 | 86302 | 19: 53439232 |
| 86304 | 19: 53484305 | 86308 | 19: 53566656 | 86309 | 19: 53572790 | 86310 | 19: 53588198 | 86314 | 19: 53698415 |
| 86318 | 19: 53748765 | 86320 | 19: 53774472 | 86321 | 19: 53778116 | 86323 | 19: 53811161 | 86333 | 19: 53948075 |
| 86337 | 19: 54028347 | 86338 | 19: 54043604 | 86341 | 19: 54053040 | 86352 | 19: 54289712 | 86353 | 19: 54295559 |
| 86355 | 19: 54301176 | 86358 | 19: 54390593 | 86371 | 19: 54622173 | 86372 | 19: 54637181 | 86379 | 19: 54710734 |
| 86383 | 19: 54755988 | 86388 | 19: 54858324 | 86393 | 19: 54978600 | 86398 | 19: 55092867 | 86403 | 19: 55229692 |
| 86406 | 19: 55265827 | 86409 | 19: 55330553 | 86413 | 19: 55427978 | 86414 | 19: 55428029 | 86422 | 19: 55580085 |
| 86427 | 19: 55698969 | 86432 | 19: 55792180 | 86433 | 19: 55793483 | 86434 | 19: 55793574 | 86436 | 19: 55827900 |
| 86437 | 19: 55830150 | 86440 | 19: 55848361 | 86441 | 19: 55848663 | 86444 | 19: 55866969 | 86449 | 19: 55906352 |
| 86452 | 19: 55911397 | 86454 | 19: 55915911 | 86455 | 19: 55917441 | 86456 | 19: 55920727 | 86458 | 19: 55922694 |
| 86462 | 19: 55926659 | 86464 | 19: 55940240 | 86471 | 19: 55985092 | 86472 | 19: 55985151 | 86473 | 19: 55985483 |
| 86474 | 19: 55986867 | 86475 | 19: 55986948 | 86477 | 19: 55987569 | 86479 | 19: 55988850 | 86480 | 19: 55988905 |
| 86481 | 19: 55988950 | 86482 | 19: 55989161 | 86483 | 19: 55989317 | 86484 | 19: 55989324 | 86486 | 19: 55993442 |
| 86489 | 19: 56009506 | 86495 | 19: 56024722 | 86500 | 19: 56044293 | 86503 | 19: 56059597 | 86512 | 19: 56100545 |
| 86516 | 19: 56104113 | 86535 | 19: 56318677 | 86536 | 19: 56320176 | 86537 | 19: 56346313 | 86538 | 19: 56346447 |
| 86539 | 19: 56348743 | 86540 | 19: 56388703 | 86544 | 19: 56447130 | 86549 | 19: 56504478 | 86550 | 19: 56506885 |
| 86551 | 19: 56506918 | 86553 | 19: 56521721 | 86554 | 19: 56527708 | 86556 | 19: 56536304 | 86558 | 19: 56542031 |
| 86559 | 19: 56544120 | 86560 | 19: 56545458 | 86561 | 19: 56546864 | 86562 | 19: 56553455 | 86571 | 19: 56792481 |
| 86573 | 19: 56794455 | 86576 | 19: 56891022 | 86579 | 19: 56962292 | 86581 | 19: 57011124 | 86582 | 19: 57012570 |
| 86591 | 19: 57196446 | 86592 | 19: 57214845 | 86593 | 19: 57259057 | 86600 | 19: 57418240 | 86608 | 19: 57566385 |
| 86631 | 19: 58091496 | 86632 | 19: 58091529 | 86634 | 19: 58127830 | 86638 | 19: 58214009 | 86639 | 19: 58218020 |
| 86645 | 19: 58304430 | 86650 | 19: 58429115 | 86659 | 19: 58594514 | 86665 | 19: 58750938 | 86673 | 19: 58918004 |
| 86688 | 19: 59274219 | 86689 | 19: 59281932 | 86711 | 19: 59592874 | 86718 | 19: 59660094 | 86724 | 19: 59743571 |
| 86727 | 19: 59784192 | 86731 | 19: 59871909 | 86732 | 19: 59872022 | 86742 | 19: 59984597 | 86756 | 19: 60173328 |
| 86768 | 19: 60319323 | 86776 | 19: 60416386 | 86781 | 19: 60493532 | 86782 | 19: 60495581 | 86793 | 19: 60643681 |
| 86795 | 19: 60660851 | 86800 | 19: 60743931 | 86802 | 19: 60816246 | 86811 | 19: 60977087 | 86814 | 19: 61065143 |
| 86818 | 19: 61145338 | 86819 | 19: 61146374 | 86822 | 19: 61175458 | 86823 | 19: 61175494 | 86824 | 19: 61175495 |
| 86825 | 19: 61175593 | 86826 | 19: 61175611 | 86834 | 19: 61242337 | 86845 | 19: 61459019 | 86846 | 19: 61474017 |
| 86848 | 19: 61529467 | 86849 | 19: 61531654 | 86850 | 19: 61531677 | 86851 | 19: 61532390 | 86852 | 19: 61533739 |
| 86854 | 19: 61538770 | 86862 | 19: 61639102 | 86865 | 19: 61681705 | 86867 | 19: 61701942 | 86868 | 19: 61709842 |
| 86874 | 19: 61794791 | 86884 | 19: 61956159 | 86885 | 19: 61968113 | 86892 | 19: 62135323 | 86904 | 19: 62322264 |
| 86915 | 19: 62611634 | 86916 | 19: 62620305 | 86919 | 19: 62738018 | 86927 | 19: 62774324 | 86928 | 19: 62785952 |
| 86942 | 19: 63013941 | 86944 | 19: 63088763 | 86946 | 19: 63091044 | 86959 | 19: 63394562 | 86960 | 19: 63394839 |
| 86965 | 20: 283185 | 86975 | 20: 495073 | 86979 | 20: 523765 | 86989 | 20: 666472 | 87009 | 20: 1104356 |
| 87012 | 20: 1164612 | 87016 | 20: 1365586 | 87019 | 20: 1476960 | 87020 | 20: 1477326 | 87025 | 20: 1585296 |
| 87031 | 20: 1627743 | 87032 | 20: 1627820 | 87033 | 20: 1628846 | 87038 | 20: 1708322 | 87040 | 20: 1708393 |
| 87051 | 20: 1881180 | 87054 | 20: 1890880 | 87058 | 20: 1964788 | 87059 | 20: 1981954 | 87060 | 20: 1985087 |
| 87062 | 20: 2008634 | 87066 | 20: 2111352 | 87071 | 20: 2174269 | 87082 | 20: 2329012 | 87084 | 20: 2363600 |
| 87090 | 20: 2486996 | 87093 | 20: 2549449 | 87094 | 20: 2549482 | 87123 | 20: 3122350 | 87125 | 20: 3132189 |
| 87126 | 20: 3144963 | 87127 | 20: 3147090 | 87128 | 20: 3149103 | 87133 | 20: 3256361 | 87141 | 20: 3434776 |
| 87143 | 20: 3442837 | 87144 | 20: 3490573 | 87153 | 20: 3643964 | 87163 | 20: 3970013 | 87184 | 20: 4419380 |
| 87186 | 20: 4422606 | 87187 | 20: 4422634 | 87189 | 20: 4471095 | 87201 | 20: 4600219 | 87353 | 20: 4974566 |
| 87356 | 20: 4978213 | 87396 | 20: 5081279 | 87397 | 20: 5081368 | 87398 | 20: 5081429 | 87411 | 20: 5256314 |
| 87424 | 20: 5427984 | 87425 | 20: 5449185 | 87427 | 20: 5456791 | 87445 | 20: 5952561 | 87471 | 20: 6124117 |
| 87472 | 20: 6127051 | 87473 | 20: 6128007 | 87474 | 20: 6129222 | 87475 | 20: 6135444 | 87478 | 20: 6222819 |
| 87491 | 20: 6538447 | 87493 | 20: 6552399 | 87502 | 20: 6787304 | 87503 | 20: 6803666 | 87504 | 20: 6804954 |
| 87505 | 20: 6809070 | 87507 | 20: 6819134 | 87508 | 20: 6824958 | 87514 | 20: 7026132 | 87515 | 20: 7028111 |
| 87527 | 20: 7184948 | 87529 | 20: 7203346 | 87530 | 20: 7228830 | 87536 | 20: 7397715 | 87538 | 20: 7452989 |
| 87556 | 20: 7937085 | 87558 | 20: 7966207 | 87565 | 20: 8099607 | 87580 | 20: 8406030 | 87588 | 20: 8596907 |
| 87591 | 20: 8676605 | 87597 | 20: 8915848 | 87599 | 20: 8976316 | 87600 | 20: 8994477 | 87603 | 20: 9008027 |
| 87604 | 20: 9008070 | 87610 | 20: 9122965 | 87611 | 20: 9185096 | 87616 | 20: 9277064 | 87617 | 20: 9284840 |
| 87618 | 20: 9286211 | 87619 | 20: 9286666 | 87620 | 20: 9297979 | 87622 | 20: 9309443 | 87627 | 20: 9387110 |
| 87635 | 20: 9562025 | 87642 | 20: 9678407 | 87643 | 20: 9699294 | 87653 | 20: 9987753 | 87657 | 20: 10105910 |
| 87670 | 20: 10568285 | 87677 | 20: 10719241 | 87691 | 20: 11104981 | 87695 | 20: 11173739 | 87708 | 20: 11384109 |
| 87709 | 20: 11423657 | 87712 | 20: 11441654 | 87719 | 20: 11603501 | 87723 | 20: 11652136 | 87726 | 20: 11746445 |
| 87727 | 20: 11748411 | 87729 | 20: 11801270 | 87732 | 20: 11873589 | 87736 | 20: 12001911 | 87745 | 20: 12195219 |
| 87751 | 20: 12336592 | 87756 | 20: 12431016 | 87759 | 20: 12454756 | 87760 | 20: 12470759 | 87770 | 20: 12619125 |
| 87776 | 20: 12725086 | 87777 | 20: 12732314 | 87787 | 20: 12872002 | 87788 | 20: 12884968 | 87792 | 20: 13038217 |
| 87800 | 20: 13212537 | 87804 | 20: 13314745 | 87806 | 20: 13360616 | 87808 | 20: 13384250 | 87809 | 20: 13411239 |
| 87820 | 20: 13846644 | 87821 | 20: 13853397 | 87827 | 20: 13967913 | 87881 | 20: 16013654 | 87890 | 20: 16311925 |
| 87891 | 20: 16312024 | 87892 | 20: 16312059 | 87893 | 20: 16312062 | 87926 | 20: 17161518 | 87940 | 20: 17529786 |
| 87946 | 20: 17718360 | 87949 | 20: 17740634 | 87950 | 20: 17772296 | 87959 | 20: 18106764 | 87966 | 20: 18199987 |
| 87974 | 20: 18560092 | 87979 | 20: 18738258 | 87981 | 20: 18767430 | 87988 | 20: 18999163 | 87998 | 20: 19219710 |
| 88010 | 20: 19525255 | 88013 | 20: 19638381 | 88024 | 20: 19910542 | 88026 | 20: 19934333 | 88029 | 20: 19951507 |
| 88030 | 20: 19985004 | 88035 | 20: 20229781 | 88049 | 20: 20735095 | 88053 | 20: 20833870 | 88054 | 20: 20847713 |
| 88055 | 20: 20852713 | 88057 | 20: 20900999 | 88058 | 20: 20916403 | 88060 | 20: 20932871 | 88062 | 20: 20985150 |
| 88069 | 20: 21165648 | 88070 | 20: 21199073 | 88076 | 20: 21442552 | 88119 | 20: 22069390 | 88123 | 20: 22130295 |
| 88158 | 20: 22204908 | 88186 | 20: 22465821 | 88204 | 20: 22827690 | 88213 | 20: 23009076 | 88214 | 20: 23038319 |
| 88215 | 20: 23039137 | 88224 | 20: 23272824 | 88234 | 20: 23534898 | 88236 | 20: 23571173 | 88237 | 20: 23574812 |
| 88238 | 20: 23575304 | 88242 | 20: 23686099 | 88243 | 20: 23692874 | 88248 | 20: 23836890 | 88249 | 20: 23837832 |
| 88250 | 20: 23838131 | 88251 | 20: 23838181 | 88252 | 20: 23841545 | 88253 | 20: 23861537 | 88254 | 20: 23861790 |
| 88255 | 20: 23862521 | 88256 | 20: 23863171 | 88257 | 20: 23864163 | 88258 | 20: 23864320 | 88260 | 20: 23868037 |
| 88261 | 20: 23872171 | 88262 | 20: 23872177 | 88263 | 20: 23873363 | 88265 | 20: 23931936 | 88266 | 20: 23934587 |
| 88267 | 20: 23936814 | 88268 | 20: 23937494 | 88269 | 20: 23940208 | 88270 | 20: 23940805 | 88271 | 20: 23948706 |
| 88272 | 20: 23950019 | 88273 | 20: 23956567 | 88274 | 20: 23967747 | 88278 | 20: 24005106 | 88283 | 20: 24183792 |
| 88289 | 20: 24300670 | 88291 | 20: 24342520 | 88301 | 20: 24551449 | 88303 | 20: 24600379 | 88304 | 20: 24644625 |
| 88314 | 20: 24840568 | 88316 | 20: 24888410 | 88336 | 20: 25687633 | 88341 | 20: 25956846 | 88353 | 20: 26326568 |
| 88377 | 20: 26904319 | 88394 | 20: 27389017 | 88396 | 20: 27389119 | 88418 | 20: 27942740 | 88423 | 20: 28062228 |
| 88424 | 20: 28062230 | 88425 | 20: 28067347 | 88435 | 20: 28291529 | 88440 | 20: 28407293 | 88449 | 20: 28594465 |
| 88452 | 20: 28714693 | 88453 | 20: 28719941 | 88454 | 20: 28719958 | 88455 | 20: 28720052 | 88456 | 20: 28726509 |
| 88457 | 20: 28726541 | 88458 | 20: 28726553 | 88459 | 20: 28726570 | 88460 | 20: 28726626 | 88468 | 20: 28835792 |
| 88474 | 20: 28977237 | 88475 | 20: 28978238 | 88488 | 20: 29362772 | 88489 | 20: 29376013 | 88491 | 20: 29447835 |
| 88508 | 20: 29990492 | 88509 | 20: 29990621 | 88514 | 20: 30118413 | 88518 | 20: 30200252 | 88520 | 20: 30212011 |
| 88532 | 20: 30669895 | 88536 | 20: 30689871 | 88539 | 20: 30825518 | 88546 | 20: 31205981 | 88547 | 20: 31211377 |
| 88548 | 20: 31243755 | 88549 | 20: 31245339 | 88558 | 20: 31456913 | 88567 | 20: 31684137 | 88570 | 20: 31752397 |
| 88576 | 20: 31881419 | 88579 | 20: 31903713 | 88582 | 20: 31972125 | 88585 | 20: 32052139 | 88588 | 20: 32101132 |
| 88597 | 20: 32409103 | 88633 | 20: 33398481 | 88637 | 20: 33502768 | 88640 | 20: 33565104 | 88641 | 20: 33569466 |
| 88648 | 20: 33719114 | 88653 | 20: 33836807 | 88657 | 20: 34001523 | 88663 | 20: 34145466 | 88666 | 20: 34184342 |
| 88669 | 20: 34272037 | 88692 | 20: 34738766 | 88694 | 20: 34781170 | 88697 | 20: 34913079 | 88714 | 20: 35421048 |
| 88721 | 20: 35551560 | 88722 | 20: 35585029 | 88723 | 20: 35610755 | 88726 | 20: 35701545 | 88728 | 20: 35715693 |
| 88736 | 20: 35846193 | 88737 | 20: 35882733 | 88741 | 20: 35994055 | 88742 | 20: 35994063 | 88743 | 20: 35996784 |
| 88762 | 20: 36319426 | 88767 | 20: 36496636 | 88776 | 20: 36648116 | 88781 | 20: 36787045 | 88782 | 20: 36834159 |
| 88783 | 20: 36858954 | 88785 | 20: 36892081 | 88788 | 20: 36968962 | 88789 | 20: 36989703 | 88791 | 20: 37042637 |
| 88794 | 20: 37085325 | 88795 | 20: 37092573 | 88797 | 20: 37131660 | 88798 | 20: 37131690 | 88799 | 20: 37136413 |
| 88804 | 20: 37341795 | 88810 | 20: 37443371 | 88811 | 20: 37443444 | 88812 | 20: 37443481 | 88813 | 20: 37473672 |
| 88815 | 20: 37479797 | 88816 | 20: 37481956 | 88827 | 20: 37694264 | 88828 | 20: 37694333 | 88831 | 20: 37811388 |
| 88832 | 20: 37811427 | 88833 | 20: 37811480 | 88834 | 20: 37811543 | 88838 | 20: 37887781 | 88845 | 20: 38177941 |
| 88846 | 20: 38189067 | 88867 | 20: 38758576 | 88881 | 20: 39088930 | 88882 | 20: 39109157 | 88887 | 20: 39238537 |
| 88888 | 20: 39245057 | 88890 | 20: 39287657 | 88893 | 20: 39383171 | 88894 | 20: 39383264 | 88898 | 20: 39445232 |
| 88904 | 20: 39475943 | 88909 | 20: 39539960 | 88911 | 20: 39555296 | 88912 | 20: 39557649 | 88924 | 20: 39810901 |
| 88925 | 20: 39863071 | 88928 | 20: 39910970 | 88930 | 20: 39965197 | 88937 | 20: 40059831 | 88941 | 20: 40215915 |
| 88955 | 20: 40683858 | 88956 | 20: 40684807 | 88957 | 20: 40695016 | 88961 | 20: 40772462 | 88966 | 20: 40834506 |
| 88968 | 20: 40945725 | 88975 | 20: 41050907 | 88976 | 20: 41050966 | 88978 | 20: 41100712 | 88984 | 20: 41160179 |
| 88986 | 20: 41192863 | 88990 | 20: 41314439 | 89010 | 20: 41798915 | 89012 | 20: 41819893 | 89013 | 20: 41823837 |
| 89016 | 20: 41845476 | 89023 | 20: 41919178 | 89028 | 20: 42044095 | 89029 | 20: 42045334 | 89032 | 20: 42085412 |
| 89035 | 20: 42133834 | 89039 | 20: 42246244 | 89042 | 20: 42293890 | 89043 | 20: 42331127 | 89047 | 20: 42565028 |
| 89061 | 20: 42942479 | 89066 | 20: 43159533 | 89079 | 20: 43561846 | 89080 | 20: 43561856 | 89101 | 20: 44025947 |
| 89102 | 20: 44026070 | 89107 | 20: 44281699 | 89108 | 20: 44301844 | 89121 | 20: 44556774 | 89122 | 20: 44574229 |
| 89123 | 20: 44591612 | 89133 | 20: 44966449 | 89134 | 20: 44969446 | 89145 | 20: 45365363 | 89186 | 20: 46291117 |
| 89194 | 20: 46578492 | 89208 | 20: 47160581 | 89209 | 20: 47160583 | 89211 | 20: 47163202 | 89212 | 20: 47163219 |
| 89243 | 20: 48405434 | 89244 | 20: 48405481 | 89245 | 20: 48405501 | 89246 | 20: 48405505 | 89247 | 20: 48405507 |
| 89248 | 20: 48405524 | 89249 | 20: 48405575 | 89251 | 20: 48474578 | 89262 | 20: 48874528 | 89264 | 20: 48881242 |
| 89265 | 20: 48881303 | 89266 | 20: 48881343 | 89267 | 20: 48901928 | 89274 | 20: 49054367 | 89297 | 20: 49861661 |
| 89298 | 20: 49913380 | 89299 | 20: 49915377 | 89300 | 20: 49918452 | 89323 | 20: 50694458 | 89328 | 20: 50806661 |
| 89338 | 20: 51034291 | 89363 | 20: 51929370 | 89385 | 20: 52736752 | 89394 | 20: 52981985 | 89401 | 20: 53204896 |
| 89405 | 20: 53347849 | 89415 | 20: 53521298 | 89416 | 20: 53526124 | 89420 | 20: 53570726 | 89428 | 20: 53840361 |
| 89442 | 20: 54395462 | 89465 | 20: 55054760 | 89466 | 20: 55055902 | 89478 | 20: 55217698 | 89485 | 20: 55403499 |
| 89500 | 20: 55900412 | 89503 | 20: 55938086 | 89522 | 20: 56361024 | 89526 | 20: 56443806 | 89530 | 20: 56569222 |
| 89532 | 20: 56609894 | 89533 | 20: 56609977 | 89536 | 20: 56692099 | 89541 | 20: 56762906 | 89570 | 20: 57486934 |
| 89571 | 20: 57487439 | 89576 | 20: 57572314 | 89584 | 20: 57778974 | 89595 | 20: 57998700 | 89596 | 20: 57998708 |
| 89597 | 20: 57998777 | 89598 | 20: 57998802 | 89599 | 20: 57998807 | 89600 | 20: 57998808 | 89601 | 20: 57998848 |
| 89602 | 20: 57998909 | 89604 | 20: 58038602 | 89605 | 20: 58038638 | 89606 | 20: 58038676 | 89610 | 20: 58171888 |
| 89622 | 20: 58370776 | 89623 | 20: 58371818 | 89628 | 20: 58425428 | 89630 | 20: 58432597 | 89638 | 20: 58662715 |
| 89652 | 20: 58839976 | 89653 | 20: 58841417 | 89661 | 20: 58985542 | 89665 | 20: 59056598 | 89668 | 20: 59062072 |
| 89675 | 20: 59262668 | 89696 | 20: 59893295 | 89698 | 20: 59918108 | 89699 | 20: 59918158 | 89700 | 20: 59918229 |
| 89702 | 20: 59984000 | 89750 | 20: 61610794 | 89751 | 20: 61610854 | 89755 | 20: 61693804 | 89767 | 20: 61962563 |
| 89773 | 20: 62086784 | 89786 | 20: 62394644 | 89790 | 20: 62513410 | 89792 | 20: 62528844 | 89794 | 20: 62575332 |
| 89799 | 20: 62653294 | 89800 | 20: 62670652 | 89808 | 20: 62848870 | 89810 | 20: 62914219 | 89811 | 20: 62937049 |
| 89812 | 20: 62942539 | 89813 | 20: 62963027 | 89815 | 20: 62971781 | 89828 | 20: 63314880 | 89830 | 20: 63382028 |
| 89831 | 20: 63384182 | 89841 | 20: 63652842 | 89843 | 20: 63711747 | 89848 | 20: 63813002 | 89857 | 20: 63923149 |
| 89858 | 20: 63934159 | 89859 | 20: 63939074 | 89860 | 20: 63939894 | 89861 | 20: 63949508 | 89862 | 20: 63950751 |
| 89863 | 20: 63952037 | 89864 | 20: 63952862 | 89865 | 20: 63954238 | 89866 | 20: 63981759 | 89871 | 20: 64108375 |
| 89891 | 20: 64483792 | 89912 | 20: 65101244 | 89919 | 20: 65253620 | 89927 | 20: 65566194 | 89928 | 20: 65578450 |
| 89929 | 20: 65592431 | 89932 | 20: 65619064 | 89934 | 20: 65691397 | 89936 | 20: 65761375 | 89937 | 20: 65773012 |
| 89938 | 20: 65786774 | 89945 | 20: 65973669 | 89950 | 20: 66089403 | 89955 | 20: 66319547 | 89959 | 20: 66370625 |
| 89960 | 20: 66372293 | 89962 | 20: 66457113 | 89963 | 20: 66464862 | 89964 | 20: 66468697 | 89967 | 20: 66551247 |
| 89968 | 20: 66556459 | 89969 | 20: 66563281 | 89970 | 20: 66563428 | 89971 | 20: 66567783 | 89972 | 20: 66568233 |
| 89973 | 20: 66626549 | 89976 | 20: 66691115 | 89978 | 20: 66696604 | 89979 | 20: 66711575 | 89980 | 20: 66711602 |
| 90001 | 20: 67153888 | 90012 | 20: 67465264 | 90016 | 20: 67521067 | 90026 | 20: 67735557 | 90027 | 20: 67739397 |
| 90028 | 20: 67743005 | 90031 | 20: 67808126 | 90032 | 20: 67819595 | 90035 | 20: 67872456 | 90039 | 20: 67995355 |
| 90041 | 20: 68014834 | 90054 | 20: 68168592 | 90062 | 20: 68323418 | 90063 | 20: 68352541 | 90071 | 20: 68463408 |
| 90072 | 20: 68503575 | 90079 | 20: 68640146 | 90087 | 20: 68746121 | 90095 | 20: 68908716 | 90102 | 20: 68990075 |
| 90129 | 20: 69518490 | 90157 | 20: 70210220 | 90182 | 20: 70941731 | 90183 | 20: 70948562 | 90184 | 20: 70949203 |
| 90185 | 20: 70954723 | 90187 | 20: 70972905 | 90188 | 20: 70973702 | 90189 | 20: 70975538 | 90193 | 20: 71082114 |
| 90194 | 20: 71097489 | 90195 | 20: 71099307 | 90196 | 20: 71104986 | 90197 | 20: 71108601 | 90198 | 20: 71110857 |
| 90199 | 20: 71113969 | 90200 | 20: 71118893 | 90201 | 20: 71119829 | 90202 | 20: 71121420 | 90203 | 20: 71122291 |
| 90204 | 20: 71124183 | 90205 | 20: 71124516 | 90206 | 20: 71127650 | 90207 | 20: 71131787 | 90208 | 20: 71139430 |
| 90209 | 20: 71141395 | 90210 | 20: 71147142 | 90211 | 20: 71147843 | 90212 | 20: 71150904 | 90213 | 20: 71153153 |
| 90214 | 20: 71154456 | 90215 | 20: 71162376 | 90216 | 20: 71172637 | 90217 | 20: 71173872 | 90218 | 20: 71177834 |
| 90219 | 20: 71180101 | 90220 | 20: 71181518 | 90221 | 20: 71183377 | 90222 | 20: 71188295 | 90223 | 20: 71195927 |
| 90224 | 20: 71201866 | 90225 | 20: 71203976 | 90226 | 20: 71206269 | 90227 | 20: 71207699 | 90228 | 20: 71212077 |
| 90229 | 20: 71232016 | 90230 | 20: 71240516 | 90231 | 20: 71290453 | 90232 | 20: 71293773 | 90233 | 20: 71295284 |
| 90234 | 20: 71300630 | 90235 | 20: 71301433 | 90236 | 20: 71313672 | 90237 | 20: 71319974 | 90238 | 20: 71320628 |
| 90239 | 20: 71349322 | 90240 | 20: 71354558 | 90241 | 20: 71355354 | 90242 | 20: 71359918 | 90243 | 20: 71360768 |
| 90244 | 20: 71378946 | 90245 | 20: 71385619 | 90246 | 20: 71386554 | 90247 | 20: 71387739 | 90248 | 20: 71388363 |
| 90249 | 20: 71393864 | 90250 | 20: 71395443 | 90251 | 20: 71396187 | 90252 | 20: 71401124 | 90254 | 20: 71402777 |
| 90255 | 20: 71403559 | 90256 | 20: 71407893 | 90257 | 20: 71417623 | 90258 | 20: 71431631 | 90259 | 20: 71442260 |
| 90260 | 20: 71444098 | 90261 | 20: 71447274 | 90262 | 20: 71448297 | 90263 | 20: 71448849 | 90264 | 20: 71450177 |
| 90265 | 20: 71450861 | 90266 | 20: 71459002 | 90267 | 20: 71472317 | 90268 | 20: 71490172 | 90269 | 20: 71491300 |
| 90272 | 20: 71606988 | 90280 | 20: 71927868 | 90305 | 21: 1606872 | 90320 | 21: 2377105 | 90330 | 21: 2684106 |
| 90336 | 21: 2874100 | 90344 | 21: 3001448 | 90366 | 21: 3866539 | 90375 | 21: 4053839 | 90377 | 21: 4055731 |
| 90378 | 21: 4060826 | 90402 | 21: 4420712 | 90413 | 21: 4948777 | 90420 | 21: 5153009 | 90425 | 21: 5327112 |
| 90428 | 21: 5382657 | 90436 | 21: 5552825 | 90437 | 21: 5575472 | 90441 | 21: 5776117 | 90449 | 21: 5865604 |
| 90453 | 21: 5946488 | 90454 | 21: 5946554 | 90455 | 21: 5961487 | 90460 | 21: 6096270 | 90469 | 21: 6466492 |
| 90471 | 21: 6515336 | 90477 | 21: 6718976 | 90479 | 21: 6821186 | 90480 | 21: 6838984 | 90481 | 21: 6864243 |
| 90488 | 21: 7110184 | 90489 | 21: 7126782 | 90506 | 21: 7582228 | 90508 | 21: 7589812 | 90509 | 21: 7590462 |
| 90520 | 21: 7863440 | 90521 | 21: 7868053 | 90524 | 21: 7907745 | 90530 | 21: 8033587 | 90534 | 21: 8074100 |
| 90535 | 21: 8074643 | 90552 | 21: 8176568 | 90554 | 21: 8190635 | 90555 | 21: 8226423 | 90562 | 21: 8310287 |
| 90604 | 21: 9465633 | 90628 | 21: 9948207 | 90632 | 21: 10010929 | 90642 | 21: 10296960 | 90647 | 21: 10509482 |
| 90653 | 21: 10609946 | 90654 | 21: 10624234 | 90671 | 21: 10957495 | 90679 | 21: 11069313 | 90693 | 21: 11440159 |
| 90694 | 21: 11445014 | 90698 | 21: 11556518 | 90703 | 21: 11654700 | 90743 | 21: 12681133 | 90749 | 21: 12760689 |
| 90754 | 21: 12855624 | 90776 | 21: 13353466 | 90780 | 21: 13514487 | 90794 | 21: 13810003 | 90798 | 21: 13905626 |
| 90805 | 21: 14029521 | 90811 | 21: 14202552 | 90823 | 21: 14517580 | 90824 | 21: 14560006 | 90834 | 21: 14713608 |
| 90840 | 21: 14945241 | 90848 | 21: 15132400 | 90849 | 21: 15138571 | 90851 | 21: 15156831 | 90852 | 21: 15156885 |
| 90853 | 21: 15156911 | 90856 | 21: 15219073 | 90859 | 21: 15326382 | 90888 | 21: 15932209 | 90894 | 21: 16294354 |
| 90896 | 21: 16311821 | 90897 | 21: 16324443 | 90901 | 21: 16376470 | 90905 | 21: 16407620 | 90906 | 21: 16426000 |
| 90908 | 21: 16455704 | 90912 | 21: 16482563 | 90914 | 21: 16507141 | 90919 | 21: 16581532 | 90920 | 21: 16583122 |
| 90922 | 21: 16605546 | 90930 | 21: 16771714 | 90932 | 21: 16775550 | 90933 | 21: 16780485 | 90940 | 21: 16994759 |
| 90945 | 21: 17105820 | 90947 | 21: 17157614 | 90967 | 21: 17696116 | 91010 | 21: 18775346 | 91011 | 21: 18792766 |
| 91012 | 21: 18796836 | 91013 | 21: 18806197 | 91014 | 21: 18807592 | 91015 | 21: 18807654 | 91018 | 21: 18889130 |
| 91019 | 21: 18889216 | 91020 | 21: 18889225 | 91021 | 21: 18897548 | 91022 | 21: 18897617 | 91023 | 21: 18897699 |
| 91024 | 21: 18906129 | 91025 | 21: 18906131 | 91035 | 21: 19086207 | 91044 | 21: 19274749 | 91045 | 21: 19274809 |
| 91049 | 21: 19328532 | 91050 | 21: 19341878 | 91058 | 21: 19448733 | 91059 | 21: 19484240 | 91061 | 21: 19509806 |
| 91065 | 21: 19547342 | 91066 | 21: 19566627 | 91067 | 21: 19571760 | 91068 | 21: 19574086 | 91069 | 21: 19574861 |
| 91071 | 21: 19581384 | 91072 | 21: 19587859 | 91074 | 21: 19591585 | 91077 | 21: 19636076 | 91078 | 21: 19650309 |
| 91079 | 21: 19744871 | 91080 | 21: 19745043 | 91081 | 21: 19765456 | 91082 | 21: 19765736 | 91083 | 21: 19765864 |
| 91084 | 21: 19765895 | 91085 | 21: 19765940 | 91088 | 21: 19791905 | 91089 | 21: 19796807 | 91090 | 21: 19800176 |
| 91092 | 21: 19819891 | 91093 | 21: 19837076 | 91120 | 21: 20576639 | 91123 | 21: 20597014 | 91124 | 21: 20611156 |
| 91129 | 21: 20643657 | 91132 | 21: 20661675 | 91137 | 21: 20707916 | 91139 | 21: 20756163 | 91140 | 21: 20767918 |
| 91148 | 21: 20994233 | 91149 | 21: 20997368 | 91150 | 21: 21002238 | 91153 | 21: 21051410 | 91154 | 21: 21051969 |
| 91155 | 21: 21079825 | 91173 | 21: 21544579 | 91175 | 21: 21547758 | 91176 | 21: 21554110 | 91178 | 21: 21572136 |
| 91189 | 21: 21882815 | 91195 | 21: 21997010 | 91197 | 21: 22018834 | 91201 | 21: 22122091 | 91202 | 21: 22130826 |
| 91203 | 21: 22132465 | 91204 | 21: 22137495 | 91205 | 21: 22138799 | 91208 | 21: 22168604 | 91217 | 21: 22354151 |
| 91222 | 21: 22473177 | 91223 | 21: 22478617 | 91232 | 21: 22695822 | 91234 | 21: 22803320 | 91240 | 21: 22922296 |
| 91244 | 21: 22980503 | 91247 | 21: 23023062 | 91249 | 21: 23081635 | 91250 | 21: 23092163 | 91254 | 21: 23177845 |
| 91255 | 21: 23177853 | 91256 | 21: 23181041 | 91257 | 21: 23190056 | 91258 | 21: 23190653 | 91259 | 21: 23195238 |
| 91260 | 21: 23211467 | 91284 | 21: 24046484 | 91288 | 21: 24273010 | 91293 | 21: 24375735 | 91347 | 21: 26116827 |
| 91361 | 21: 26448325 | 91368 | 21: 26581951 | 91371 | 21: 26637918 | 91383 | 21: 26979057 | 91384 | 21: 26999182 |
| 91385 | 21: 26999251 | 91397 | 21: 27503033 | 91398 | 21: 27508911 | 91407 | 21: 27743344 | 91427 | 21: 28338007 |
| 91441 | 21: 28917960 | 91443 | 21: 28948600 | 91444 | 21: 28961889 | 91448 | 21: 29052667 | 91461 | 21: 29439618 |
| 91465 | 21: 29553919 | 91471 | 21: 29725150 | 91488 | 21: 30205371 | 91489 | 21: 30225427 | 91493 | 21: 30359230 |
| 91494 | 21: 30364775 | 91495 | 21: 30364784 | 91497 | 21: 30380463 | 91498 | 21: 30383546 | 91500 | 21: 30415216 |
| 91504 | 21: 30548247 | 91507 | 21: 30574539 | 91508 | 21: 30583259 | 91512 | 21: 30655015 | 91513 | 21: 30665920 |
| 91514 | 21: 30666267 | 91518 | 21: 30752337 | 91521 | 21: 30836177 | 91525 | 21: 31068352 | 91526 | 21: 31085819 |
| 91528 | 21: 31169053 | 91538 | 21: 31453639 | 91539 | 21: 31464548 | 91540 | 21: 31470928 | 91541 | 21: 31485138 |
| 91542 | 21: 31493718 | 91543 | 21: 31496586 | 91544 | 21: 31499460 | 91545 | 21: 31522156 | 91546 | 21: 31525621 |
| 91547 | 21: 31534000 | 91548 | 21: 31541459 | 91549 | 21: 31545947 | 91550 | 21: 31553803 | 91551 | 21: 31604982 |
| 91553 | 21: 31720553 | 91554 | 21: 31725673 | 91555 | 21: 31734161 | 91556 | 21: 31736971 | 91557 | 21: 31754761 |
| 91558 | 21: 31758189 | 91559 | 21: 31781060 | 91560 | 21: 31791229 | 91563 | 21: 31955982 | 91574 | 21: 32336068 |
| 91575 | 21: 32364556 | 91578 | 21: 32403515 | 91584 | 21: 32692535 | 91585 | 21: 32705474 | 91589 | 21: 32783989 |
| 91590 | 21: 32814280 | 91598 | 21: 33110998 | 91602 | 21: 33166758 | 91603 | 21: 33168203 | 91604 | 21: 33169028 |
| 91605 | 21: 33170204 | 91606 | 21: 33170309 | 91607 | 21: 33171324 | 91608 | 21: 33171538 | 91610 | 21: 33205680 |
| 91611 | 21: 33205893 | 91612 | 21: 33208660 | 91614 | 21: 33215961 | 91615 | 21: 33225284 | 91622 | 21: 33373120 |
| 91623 | 21: 33373124 | 91627 | 21: 33449736 | 91628 | 21: 33458505 | 91629 | 21: 33459344 | 91630 | 21: 33461031 |
| 91631 | 21: 33461192 | 91632 | 21: 33461449 | 91633 | 21: 33465757 | 91634 | 21: 33465758 | 91635 | 21: 33466480 |
| 91636 | 21: 33469426 | 91637 | 21: 33469555 | 91638 | 21: 33469624 | 91639 | 21: 33469729 | 91640 | 21: 33469770 |
| 91641 | 21: 33469982 | 91642 | 21: 33483461 | 91643 | 21: 33487373 | 91645 | 21: 33529381 | 91648 | 21: 33547120 |
| 91650 | 21: 33571261 | 91652 | 21: 33652175 | 91654 | 21: 33661162 | 91670 | 21: 33947582 | 91672 | 21: 33991645 |
| 91675 | 21: 34024711 | 91676 | 21: 34044738 | 91677 | 21: 34049707 | 91680 | 21: 34087450 | 91691 | 21: 34273652 |
| 91692 | 21: 34276064 | 91694 | 21: 34296572 | 91703 | 21: 34434123 | 91705 | 21: 34478879 | 91706 | 21: 34487765 |
| 91707 | 21: 34490950 | 91712 | 21: 34556901 | 91713 | 21: 34556925 | 91714 | 21: 34557109 | 91715 | 21: 34585611 |
| 91718 | 21: 34637208 | 91719 | 21: 34637260 | 91731 | 21: 34938997 | 91732 | 21: 34939108 | 91737 | 21: 35075467 |
| 91741 | 21: 35177228 | 91742 | 21: 35177300 | 91752 | 21: 35387396 | 91753 | 21: 35391211 | 91755 | 21: 35545267 |
| 91761 | 21: 35621906 | 91787 | 21: 36004086 | 91801 | 21: 36349981 | 91807 | 21: 36467140 | 91844 | 21: 37588472 |
| 91855 | 21: 37884949 | 91856 | 21: 37889952 | 91863 | 21: 38352155 | 91866 | 21: 38488688 | 91901 | 21: 39497698 |
| 91902 | 21: 39497796 | 91927 | 21: 40117870 | 91928 | 21: 40123103 | 91930 | 21: 40267167 | 91934 | 21: 40321474 |
| 91950 | 21: 40777585 | 91955 | 21: 41031761 | 91956 | 21: 41063587 | 91958 | 21: 41103681 | 91959 | 21: 41189588 |
| 91960 | 21: 41190356 | 91961 | 21: 41230647 | 91963 | 21: 41268397 | 91968 | 21: 41377730 | 91969 | 21: 41377811 |
| 91971 | 21: 41381520 | 91972 | 21: 41381521 | 91973 | 21: 41381562 | 91979 | 21: 41500659 | 91980 | 21: 41500733 |
| 91988 | 21: 41683205 | 91995 | 21: 41951773 | 91999 | 21: 41985647 | 92001 | 21: 42021205 | 92005 | 21: 42056585 |
| 92006 | 21: 42056665 | 92011 | 21: 42193358 | 92015 | 21: 42263974 | 92025 | 21: 42613095 | 92026 | 21: 42613704 |
| 92027 | 21: 42615428 | 92028 | 21: 42632006 | 92036 | 21: 42789940 | 92037 | 21: 42789959 | 92038 | 21: 42794024 |
| 92042 | 21: 42894892 | 92043 | 21: 42894971 | 92044 | 21: 42906559 | 92048 | 21: 42991321 | 92060 | 21: 43369442 |
| 92073 | 21: 43694807 | 92090 | 21: 44134802 | 92093 | 21: 44170697 | 92094 | 21: 44193483 | 92095 | 21: 44198391 |
| 92096 | 21: 44204274 | 92097 | 21: 44204999 | 92098 | 21: 44208959 | 92103 | 21: 44374543 | 92108 | 21: 44464711 |
| 92111 | 21: 44510750 | 92112 | 21: 44558742 | 92119 | 21: 44670112 | 92124 | 21: 44711056 | 92125 | 21: 44711119 |
| 92128 | 21: 44747742 | 92131 | 21: 44794407 | 92137 | 21: 44874950 | 92146 | 21: 45364186 | 92153 | 21: 45617906 |
| 92162 | 21: 45840926 | 92168 | 21: 46169637 | 92177 | 21: 46371720 | 92178 | 21: 46372978 | 92179 | 21: 46375756 |
| 92180 | 21: 46377528 | 92181 | 21: 46378483 | 92182 | 21: 46399567 | 92183 | 21: 46411679 | 92207 | 21: 47030345 |
| 92209 | 21: 47120773 | 92210 | 21: 47135961 | 92235 | 21: 47300254 | 92252 | 21: 47315681 | 92255 | 21: 47399940 |
| 92260 | 21: 47464301 | 92272 | 21: 47936288 | 92293 | 21: 48482014 | 92308 | 21: 48897167 | 92315 | 21: 49088678 |
| 92316 | 21: 49165291 | 92317 | 21: 49165372 | 92325 | 21: 49212043 | 92348 | 21: 49756597 | 92349 | 21: 49773549 |
| 92414 | 21: 51757967 | 92423 | 21: 52002988 | 92430 | 21: 52140964 | 92438 | 21: 52391191 | 92447 | 21: 52590908 |
| 92459 | 21: 53100162 | 92467 | 21: 53274187 | 92468 | 21: 53295524 | 92469 | 21: 53295551 | 92473 | 21: 53382594 |
| 92494 | 21: 53955771 | 92511 | 21: 54486141 | 92519 | 21: 54707788 | 92533 | 21: 54895636 | 92534 | 21: 54906059 |
| 92535 | 21: 54910848 | 92536 | 21: 54911588 | 92537 | 21: 54911598 | 92538 | 21: 54919544 | 92540 | 21: 54956256 |
| 92544 | 21: 55027058 | 92549 | 21: 55139938 | 92551 | 21: 55157787 | 92553 | 21: 55268142 | 92555 | 21: 55276126 |
| 92559 | 21: 55336100 | 92567 | 21: 55534889 | 92600 | 21: 56030046 | 92609 | 21: 56102239 | 92611 | 21: 56151393 |
| 92612 | 21: 56165597 | 92613 | 21: 56174882 | 92614 | 21: 56184939 | 92616 | 21: 56194199 | 92617 | 21: 56195714 |
| 92618 | 21: 56211969 | 92625 | 21: 56552387 | 92627 | 21: 56570919 | 92637 | 21: 56804066 | 92689 | 21: 57070861 |
| 92690 | 21: 57070865 | 92713 | 21: 57163461 | 92714 | 21: 57163519 | 92730 | 21: 57357698 | 92737 | 21: 57514823 |
| 92740 | 21: 57650013 | 92744 | 21: 57789826 | 92745 | 21: 57815184 | 92746 | 21: 57841247 | 92752 | 21: 58086488 |
| 92760 | 21: 58328406 | 92761 | 21: 58335169 | 92762 | 21: 58348854 | 92765 | 21: 58457450 | 92769 | 21: 58542608 |
| 92777 | 21: 58649278 | 92778 | 21: 58649996 | 92779 | 21: 58650822 | 92780 | 21: 58651655 | 92781 | 21: 58651789 |
| 92782 | 21: 58653170 | 92783 | 21: 58653759 | 92784 | 21: 58653796 | 92785 | 21: 58654257 | 92786 | 21: 58657343 |
| 92787 | 21: 58730952 | 92792 | 21: 58844584 | 92794 | 21: 58897764 | 92812 | 21: 59280395 | 92813 | 21: 59290376 |
| 92816 | 21: 59410654 | 92817 | 21: 59450928 | 92821 | 21: 59494769 | 92838 | 21: 59798491 | 92846 | 21: 59885566 |
| 92847 | 21: 59892560 | 92854 | 21: 59967817 | 92857 | 21: 59995456 | 92858 | 21: 60034051 | 92860 | 21: 60070408 |
| 92864 | 21: 60128571 | 92867 | 21: 60147487 | 92870 | 21: 60222745 | 92872 | 21: 60258085 | 92875 | 21: 60285746 |
| 92878 | 21: 60309341 | 92879 | 21: 60318597 | 92880 | 21: 60364407 | 92887 | 21: 60531623 | 92888 | 21: 60531761 |
| 92914 | 21: 60963159 | 92929 | 21: 61332759 | 92935 | 21: 61617246 | 92936 | 21: 61637616 | 92937 | 21: 61639799 |
| 92938 | 21: 61640498 | 92957 | 21: 62025836 | 92973 | 21: 62390233 | 93001 | 21: 63134292 | 93003 | 21: 63222235 |
| 93004 | 21: 63258242 | 93010 | 21: 63480195 | 93017 | 21: 63603801 | 93024 | 21: 63831078 | 93029 | 21: 63935578 |
| 93046 | 21: 64333571 | 93050 | 21: 64428779 | 93051 | 21: 64450577 | 93052 | 21: 64452975 | 93065 | 21: 64633547 |
| 93070 | 21: 64699689 | 93077 | 21: 64841812 | 93078 | 21: 64841843 | 93079 | 21: 64841865 | 93080 | 21: 64850271 |
| 93084 | 21: 64922535 | 93086 | 21: 64988072 | 93088 | 21: 65014550 | 93089 | 21: 65027672 | 93090 | 21: 65029870 |
| 93091 | 21: 65051618 | 93092 | 21: 65052179 | 93093 | 21: 65052717 | 93094 | 21: 65054313 | 93095 | 21: 65055762 |
| 93096 | 21: 65056739 | 93097 | 21: 65060441 | 93098 | 21: 65061116 | 93099 | 21: 65067323 | 93100 | 21: 65073410 |
| 93101 | 21: 65073892 | 93102 | 21: 65075325 | 93103 | 21: 65079623 | 93104 | 21: 65080143 | 93105 | 21: 65082481 |
| 93114 | 21: 65232598 | 93167 | 21: 66311776 | 93183 | 21: 66723461 | 93189 | 21: 66870945 | 93190 | 21: 66929334 |
| 93191 | 21: 66932880 | 93192 | 21: 66934300 | 93193 | 21: 66936241 | 93194 | 21: 66938188 | 93195 | 21: 66942278 |
| 93197 | 21: 66945357 | 93204 | 21: 67094952 | 93208 | 21: 67149363 | 93215 | 21: 67449798 | 93221 | 21: 67552612 |
| 93231 | 21: 67865058 | 93232 | 21: 67876373 | 93240 | 21: 67965438 | 93251 | 21: 68196501 | 93252 | 21: 68202138 |
| 93253 | 21: 68202185 | 93255 | 21: 68230479 | 93258 | 21: 68286931 | 93260 | 21: 68335052 | 93268 | 21: 68506930 |
| 93270 | 21: 68519125 | 93271 | 21: 68522170 | 93272 | 21: 68529965 | 93273 | 21: 68530561 | 93274 | 21: 68530572 |
| 93276 | 21: 68537086 | 93280 | 21: 68668451 | 93294 | 21: 69004265 | 93301 | 21: 69165607 | 93304 | 21: 69229046 |
| 93308 | 21: 69352283 | 93309 | 21: 69352557 | 93310 | 21: 69353990 | 93312 | 21: 69409067 | 93314 | 21: 69440442 |
| 93315 | 21: 69440464 | 93316 | 21: 69440466 | 93317 | 21: 69442039 | 93320 | 21: 69557781 | 93321 | 21: 69574694 |
| 93322 | 21: 69582716 | 93324 | 21: 69629308 | 93325 | 21: 69642755 | 93326 | 21: 69660286 | 93331 | 21: 69725960 |
| 93332 | 21: 69764707 | 93333 | 21: 69765916 | 93334 | 21: 69766081 | 93335 | 21: 69770916 | 93345 | 22: 378758 |
| 93353 | 22: 651171 | 93363 | 22: 912338 | 93366 | 22: 940171 | 93367 | 22: 940370 | 93395 | 22: 1560877 |
| 93401 | 22: 1738773 | 93402 | 22: 1738804 | 93403 | 22: 1738805 | 93404 | 22: 1746536 | 93414 | 22: 1927654 |
| 93432 | 22: 2329624 | 93449 | 22: 2751520 | 93450 | 22: 2752726 | 93456 | 22: 2877258 | 93457 | 22: 2892709 |
| 93465 | 22: 3056974 | 93471 | 22: 3197001 | 93473 | 22: 3220010 | 93475 | 22: 3269948 | 93482 | 22: 3359434 |
| 93493 | 22: 3596980 | 93499 | 22: 3798423 | 93501 | 22: 3834731 | 93504 | 22: 3914101 | 93506 | 22: 3960951 |
| 93529 | 22: 4497784 | 93540 | 22: 4729907 | 93546 | 22: 4842711 | 93551 | 22: 4922676 | 93556 | 22: 5030837 |
| 93559 | 22: 5120220 | 93561 | 22: 5160439 | 93562 | 22: 5165375 | 93564 | 22: 5179197 | 93575 | 22: 5341524 |
| 93576 | 22: 5345473 | 93577 | 22: 5391435 | 93584 | 22: 5485152 | 93595 | 22: 5647094 | 93597 | 22: 5693798 |
| 93598 | 22: 5721636 | 93600 | 22: 5764445 | 93605 | 22: 5883723 | 93612 | 22: 5950788 | 93616 | 22: 6008417 |
| 93617 | 22: 6017167 | 93618 | 22: 6052161 | 93622 | 22: 6199633 | 93627 | 22: 6252438 | 93630 | 22: 6293377 |
| 93636 | 22: 6370667 | 93643 | 22: 6488129 | 93645 | 22: 6498701 | 93657 | 22: 6784495 | 93660 | 22: 6839952 |
| 93671 | 22: 6996393 | 93685 | 22: 7428239 | 93686 | 22: 7434501 | 93697 | 22: 7651542 | 93702 | 22: 7841139 |
| 93703 | 22: 7908696 | 93723 | 22: 8411667 | 93735 | 22: 8761701 | 93772 | 22: 9753217 | 93774 | 22: 9795347 |
| 93783 | 22: 10006268 | 93784 | 22: 10006273 | 93785 | 22: 10006376 | 93787 | 22: 10035911 | 93789 | 22: 10081106 |
| 93790 | 22: 10081118 | 93791 | 22: 10081165 | 93792 | 22: 10081187 | 93793 | 22: 10081208 | 93794 | 22: 10081215 |
| 93795 | 22: 10081251 | 93796 | 22: 10081295 | 93801 | 22: 10172170 | 93804 | 22: 10236047 | 93809 | 22: 10290595 |
| 93810 | 22: 10345715 | 93812 | 22: 10401777 | 93836 | 22: 11003544 | 93841 | 22: 11122201 | 93850 | 22: 11391522 |
| 93858 | 22: 11578648 | 93860 | 22: 11718954 | 93861 | 22: 11720712 | 93865 | 22: 11758613 | 93867 | 22: 11831403 |
| 93868 | 22: 11832624 | 93869 | 22: 11889790 | 93870 | 22: 11889853 | 93871 | 22: 11890006 | 93872 | 22: 11890127 |
| 93878 | 22: 11973585 | 93879 | 22: 11977784 | 93880 | 22: 11977859 | 93881 | 22: 11978609 | 93882 | 22: 11978855 |
| 93883 | 22: 11978903 | 93886 | 22: 12019884 | 93892 | 22: 12195260 | 93897 | 22: 12303365 | 93900 | 22: 12377148 |
| 93901 | 22: 12430483 | 93902 | 22: 12436371 | 93904 | 22: 12451350 | 93907 | 22: 12503200 | 93908 | 22: 12506458 |
| 93917 | 22: 12662972 | 93921 | 22: 12724433 | 93930 | 22: 12923416 | 93936 | 22: 13091338 | 93943 | 22: 13359849 |
| 93951 | 22: 13559915 | 93952 | 22: 13582865 | 93969 | 22: 14142677 | 93970 | 22: 14145371 | 93973 | 22: 14181865 |
| 93976 | 22: 14213464 | 93978 | 22: 14312375 | 93984 | 22: 14511531 | 93985 | 22: 14512264 | 93986 | 22: 14516780 |
| 93987 | 22: 14516787 | 93988 | 22: 14518692 | 93989 | 22: 14519180 | 94006 | 22: 15072933 | 94008 | 22: 15123459 |
| 94019 | 22: 15471657 | 94022 | 22: 15504111 | 94027 | 22: 15605731 | 94051 | 22: 16322403 | 94052 | 22: 16322451 |
| 94053 | 22: 16322488 | 94054 | 22: 16353524 | 94062 | 22: 16546381 | 94063 | 22: 16548801 | 94064 | 22: 16561111 |
| 94065 | 22: 16567113 | 94066 | 22: 16567586 | 94067 | 22: 16568933 | 94073 | 22: 16666585 | 94077 | 22: 16725586 |
| 94078 | 22: 16728120 | 94080 | 22: 16741450 | 94082 | 22: 16757417 | 94083 | 22: 16773003 | 94084 | 22: 16802200 |
| 94085 | 22: 16804567 | 94100 | 22: 17418863 | 94113 | 22: 17718315 | 94116 | 22: 17791469 | 94123 | 22: 17937879 |
| 94125 | 22: 17953330 | 94130 | 22: 18035176 | 94132 | 22: 18061536 | 94151 | 22: 18541264 | 94156 | 22: 18614346 |
| 94160 | 22: 18696586 | 94164 | 22: 18832117 | 94175 | 22: 19093982 | 94177 | 22: 19212118 | 94185 | 22: 19376422 |
| 94215 | 22: 20299573 | 94221 | 22: 20368774 | 94234 | 22: 20830719 | 94259 | 22: 21264256 | 94260 | 22: 21281505 |
| 94262 | 22: 21343700 | 94270 | 22: 21553615 | 94272 | 22: 21581948 | 94275 | 22: 21638280 | 94282 | 22: 21687964 |
| 94284 | 22: 21691311 | 94285 | 22: 21691369 | 94286 | 22: 21696971 | 94287 | 22: 21697320 | 94289 | 22: 21780680 |
| 94295 | 22: 21936689 | 94298 | 22: 22018100 | 94301 | 22: 22096896 | 94302 | 22: 22130081 | 94304 | 22: 22207036 |
| 94305 | 22: 22280513 | 94306 | 22: 22280554 | 94312 | 22: 22511637 | 94321 | 22: 22847965 | 94325 | 22: 22910885 |
| 94328 | 22: 22978479 | 94390 | 22: 24919962 | 94419 | 22: 25887179 | 94426 | 22: 26004558 | 94427 | 22: 26004600 |
| 94428 | 22: 26006402 | 94429 | 22: 26028973 | 94431 | 22: 26048198 | 94432 | 22: 26048256 | 94433 | 22: 26055574 |
| 94437 | 22: 26092272 | 94438 | 22: 26092293 | 94442 | 22: 26179382 | 94443 | 22: 26199906 | 94446 | 22: 26342475 |
| 94474 | 22: 27220605 | 94480 | 22: 27400091 | 94490 | 22: 27635934 | 94507 | 22: 28135962 | 94512 | 22: 28188835 |
| 94514 | 22: 28211359 | 94518 | 22: 28297855 | 94523 | 22: 28404142 | 94524 | 22: 28404219 | 94529 | 22: 28481900 |
| 94531 | 22: 28515749 | 94533 | 22: 28587264 | 94534 | 22: 28588309 | 94539 | 22: 28733810 | 94541 | 22: 28784852 |
| 94542 | 22: 28784985 | 94543 | 22: 28792168 | 94545 | 22: 28835841 | 94546 | 22: 28835907 | 94547 | 22: 28835973 |
| 94548 | 22: 28840124 | 94549 | 22: 28840129 | 94550 | 22: 28843084 | 94551 | 22: 28844382 | 94552 | 22: 28845975 |
| 94554 | 22: 28862947 | 94555 | 22: 28938955 | 94557 | 22: 29005992 | 94566 | 22: 29299265 | 94581 | 22: 29705813 |
| 94582 | 22: 29709388 | 94583 | 22: 29712615 | 94604 | 22: 30274003 | 94607 | 22: 30417109 | 94608 | 22: 30436771 |
| 94610 | 22: 30515471 | 94624 | 22: 30808716 | 94638 | 22: 31133292 | 94639 | 22: 31135175 | 94640 | 22: 31141034 |
| 94661 | 22: 31835374 | 94662 | 22: 31861512 | 94663 | 22: 31891766 | 94666 | 22: 32022974 | 94673 | 22: 32156119 |
| 94674 | 22: 32186880 | 94675 | 22: 32233824 | 94676 | 22: 32246231 | 94679 | 22: 32399020 | 94685 | 22: 32572065 |
| 94688 | 22: 32679434 | 94698 | 22: 32982493 | 94700 | 22: 32993742 | 94701 | 22: 33017003 | 94702 | 22: 33053278 |
| 94708 | 22: 33142515 | 94728 | 22: 33935530 | 94729 | 22: 33944757 | 94733 | 22: 33983138 | 94768 | 22: 34968813 |
| 94771 | 22: 35012254 | 94774 | 22: 35039773 | 94781 | 22: 35173959 | 94791 | 22: 35329495 | 94793 | 22: 35423874 |
| 94796 | 22: 35459751 | 94797 | 22: 35460784 | 94799 | 22: 35523854 | 94810 | 22: 35736939 | 94812 | 22: 35774994 |
| 94815 | 22: 35862159 | 94816 | 22: 35890192 | 94817 | 22: 35901222 | 94826 | 22: 36031535 | 94837 | 22: 36381197 |
| 94844 | 22: 36496828 | 94846 | 22: 36551821 | 94848 | 22: 36609940 | 94856 | 22: 36967669 | 94865 | 22: 37191405 |
| 94866 | 22: 37210076 | 94872 | 22: 37384626 | 94873 | 22: 37391008 | 94876 | 22: 37473284 | 94883 | 22: 37617112 |
| 94884 | 22: 37620082 | 94885 | 22: 37622201 | 94886 | 22: 37624592 | 94887 | 22: 37627226 | 94888 | 22: 37628993 |
| 94889 | 22: 37635996 | 94890 | 22: 37636848 | 94891 | 22: 37637997 | 94892 | 22: 37640217 | 94893 | 22: 37653222 |
| 94917 | 22: 38367562 | 94919 | 22: 38384223 | 94924 | 22: 38493420 | 94928 | 22: 38548453 | 94934 | 22: 38617876 |
| 94944 | 22: 38801073 | 94955 | 22: 39102590 | 94963 | 22: 39227562 | 94964 | 22: 39234430 | 94965 | 22: 39240885 |
| 94977 | 22: 39399375 | 94978 | 22: 39399383 | 94996 | 22: 39664313 | 95001 | 22: 39736141 | 95002 | 22: 39736166 |
| 95003 | 22: 39742270 | 95005 | 22: 39764001 | 95007 | 22: 39806230 | 95022 | 22: 40159960 | 95028 | 22: 40412439 |
| 95030 | 22: 40474842 | 95031 | 22: 40497320 | 95033 | 22: 40497948 | 95036 | 22: 40553964 | 95043 | 22: 40698664 |
| 95044 | 22: 40699122 | 95046 | 22: 40709407 | 95064 | 22: 41331703 | 95077 | 22: 41673525 | 95078 | 22: 41697991 |
| 95104 | 22: 42421167 | 95108 | 22: 42552768 | 95118 | 22: 42753833 | 95120 | 22: 42860068 | 95131 | 22: 43070171 |
| 95133 | 22: 43103372 | 95135 | 22: 43152197 | 95137 | 22: 43171695 | 95139 | 22: 43185881 | 95142 | 22: 43216398 |
| 95145 | 22: 43241133 | 95148 | 22: 43280264 | 95151 | 22: 43327456 | 95152 | 22: 43328177 | 95158 | 22: 43532708 |
| 95162 | 22: 43617671 | 95170 | 22: 43831970 | 95172 | 22: 43860815 | 95190 | 22: 44240079 | 95191 | 22: 44242443 |
| 95195 | 22: 44303591 | 95212 | 22: 44704423 | 95217 | 22: 44800260 | 95225 | 22: 45101725 | 95232 | 22: 45343274 |
| 95245 | 22: 45656061 | 95247 | 22: 45691255 | 95252 | 22: 45783940 | 95255 | 22: 45826883 | 95270 | 22: 46040006 |
| 95273 | 22: 46071530 | 95291 | 22: 46619809 | 95296 | 22: 46740639 | 95298 | 22: 46748886 | 95311 | 22: 46981263 |
| 95312 | 22: 46981314 | 95315 | 22: 47033448 | 95318 | 22: 47057561 | 95331 | 22: 47235742 | 95333 | 22: 47261646 |
| 95337 | 22: 47402038 | 95342 | 22: 47467741 | 95349 | 22: 47617100 | 95352 | 22: 47677699 | 95356 | 22: 47817718 |
| 95361 | 22: 48000435 | 95364 | 22: 48019220 | 95369 | 22: 48066613 | 95370 | 22: 48091175 | 95371 | 22: 48107831 |
| 95372 | 22: 48170389 | 95373 | 22: 48171831 | 95380 | 22: 48435818 | 95381 | 22: 48449692 | 95386 | 22: 48687235 |
| 95388 | 22: 48709706 | 95391 | 22: 48761536 | 95395 | 22: 48961009 | 95396 | 22: 48979936 | 95397 | 22: 48988156 |
| 95398 | 22: 49025901 | 95399 | 22: 49040313 | 95400 | 22: 49041656 | 95405 | 22: 49152003 | 95407 | 22: 49218903 |
| 95411 | 22: 49396397 | 95412 | 22: 49411257 | 95413 | 22: 49416701 | 95417 | 22: 49475649 | 95422 | 22: 49751795 |
| 95427 | 22: 49845416 | 95428 | 22: 49855390 | 95429 | 22: 49862171 | 95430 | 22: 49874759 | 95437 | 22: 50031711 |
| 95443 | 22: 50110984 | 95444 | 22: 50127865 | 95446 | 22: 50174136 | 95448 | 22: 50267951 | 95453 | 22: 50398849 |
| 95463 | 22: 50615575 | 95464 | 22: 50626022 | 95467 | 22: 50669330 | 95468 | 22: 50669331 | 95471 | 22: 50732557 |
| 95472 | 22: 50752636 | 95475 | 22: 50849444 | 95488 | 22: 51284290 | 95490 | 22: 51317516 | 95495 | 22: 51400864 |
| 95502 | 22: 51573666 | 95509 | 22: 51800322 | 95517 | 22: 51985899 | 95518 | 22: 51994258 | 95521 | 22: 52090420 |
| 95522 | 22: 52090421 | 95523 | 22: 52090571 | 95526 | 22: 52113515 | 95528 | 22: 52142954 | 95535 | 22: 52398536 |
| 95536 | 22: 52409661 | 95545 | 22: 52642109 | 95547 | 22: 52689484 | 95555 | 22: 52876456 | 95559 | 22: 52889478 |
| 95562 | 22: 52940887 | 95567 | 22: 52998347 | 95568 | 22: 52998797 | 95569 | 22: 52998800 | 95570 | 22: 52999211 |
| 95571 | 22: 52999397 | 95573 | 22: 53017868 | 95586 | 22: 53333639 | 95595 | 22: 53587524 | 95596 | 22: 53630193 |
| 95597 | 22: 53632109 | 95599 | 22: 53667747 | 95601 | 22: 53701852 | 95603 | 22: 53750298 | 95604 | 22: 53771417 |
| 95608 | 22: 53814947 | 95609 | 22: 53815430 | 95610 | 22: 53823185 | 95617 | 22: 53952061 | 95625 | 22: 54050342 |
| 95626 | 22: 54084951 | 95634 | 22: 54271229 | 95636 | 22: 54325220 | 95637 | 22: 54325294 | 95638 | 22: 54326413 |
| 95639 | 22: 54326417 | 95640 | 22: 54326525 | 95652 | 22: 54582223 | 95654 | 22: 54586616 | 95655 | 22: 54590257 |
| 95656 | 22: 54591674 | 95657 | 22: 54601062 | 95659 | 22: 54619999 | 95660 | 22: 54656963 | 95661 | 22: 54657085 |
| 95681 | 22: 55222425 | 95682 | 22: 55249217 | 95684 | 22: 55290303 | 95685 | 22: 55290330 | 95687 | 22: 55290410 |
| 95688 | 22: 55290479 | 95689 | 22: 55290914 | 95690 | 22: 55291002 | 95691 | 22: 55291168 | 95692 | 22: 55291236 |
| 95693 | 22: 55291381 | 95694 | 22: 55291382 | 95697 | 22: 55338384 | 95699 | 22: 55379431 | 95700 | 22: 55413027 |
| 95705 | 22: 55446332 | 95706 | 22: 55455982 | 95709 | 22: 55483225 | 95713 | 22: 55579849 | 95725 | 22: 55804900 |
| 95728 | 22: 55847268 | 95733 | 22: 55887174 | 95734 | 22: 55887346 | 95735 | 22: 55887394 | 95736 | 22: 55887397 |
| 95737 | 22: 55887398 | 95738 | 22: 55887409 | 95739 | 22: 55887425 | 95740 | 22: 55887575 | 95741 | 22: 55888374 |
| 95746 | 22: 55952909 | 95759 | 22: 56301696 | 95761 | 22: 56325207 | 95762 | 22: 56325210 | 95763 | 22: 56325225 |
| 95764 | 22: 56325235 | 95765 | 22: 56325272 | 95766 | 22: 56325600 | 95767 | 22: 56325606 | 95769 | 22: 56325745 |
| 95770 | 22: 56325754 | 95771 | 22: 56326527 | 95772 | 22: 56329687 | 95773 | 22: 56329721 | 95774 | 22: 56332166 |
| 95775 | 22: 56340185 | 95777 | 22: 56377918 | 95778 | 22: 56388281 | 95780 | 22: 56396437 | 95791 | 22: 56484402 |
| 95792 | 22: 56513273 | 95797 | 22: 56546272 | 95800 | 22: 56616418 | 95801 | 22: 56632616 | 95822 | 22: 57017818 |
| 95823 | 22: 57017940 | 95825 | 22: 57074332 | 95827 | 22: 57132556 | 95830 | 22: 57180726 | 95831 | 22: 57198542 |
| 95832 | 22: 57228439 | 95833 | 22: 57257734 | 95834 | 22: 57268915 | 95840 | 22: 57427960 | 95841 | 22: 57434193 |
| 95843 | 22: 57453058 | 95846 | 22: 57478438 | 95847 | 22: 57511375 | 95851 | 22: 57532676 | 95855 | 22: 57579073 |
| 95857 | 22: 57629519 | 95858 | 22: 57638240 | 95869 | 22: 57759457 | 95870 | 22: 57764469 | 95871 | 22: 57766664 |
| 95872 | 22: 57792445 | 95890 | 22: 58069483 | 95891 | 22: 58075933 | 95892 | 22: 58086195 | 95905 | 22: 58427446 |
| 95907 | 22: 58446882 | 95911 | 22: 58506677 | 95925 | 22: 58804900 | 95938 | 22: 59105276 | 95939 | 22: 59111787 |
| 95945 | 22: 59165452 | 95947 | 22: 59174986 | 95950 | 22: 59237930 | 95961 | 22: 59518872 | 95966 | 22: 59625926 |
| 95978 | 22: 59729840 | 95982 | 22: 59783092 | 95983 | 22: 59831717 | 95989 | 22: 59928044 | 96005 | 22: 60263301 |
| 96007 | 22: 60314302 | 96008 | 22: 60344480 | 96009 | 22: 60369311 | 96013 | 22: 60433161 | 96014 | 22: 60450289 |
| 96015 | 22: 60450822 | 96016 | 22: 60450826 | 96021 | 22: 60583428 | 96024 | 22: 60664148 | 96028 | 23: 98715 |
| 96048 | 23: 908722 | 96059 | 23: 1233960 | 96067 | 23: 1413088 | 96081 | 23: 1806494 | 96089 | 23: 2027290 |
| 96091 | 23: 2085668 | 96102 | 23: 2295458 | 96103 | 23: 2327306 | 96109 | 23: 2490639 | 96115 | 23: 2633185 |
| 96119 | 23: 2653923 | 96121 | 23: 2672734 | 96122 | 23: 2681218 | 96131 | 23: 2932961 | 96132 | 23: 2932963 |
| 96133 | 23: 2933026 | 96134 | 23: 2933089 | 96135 | 23: 2933119 | 96136 | 23: 2943350 | 96137 | 23: 2944259 |
| 96138 | 23: 2945377 | 96147 | 23: 3182108 | 96150 | 23: 3278552 | 96152 | 23: 3363897 | 96154 | 23: 3397926 |
| 96155 | 23: 3401808 | 96156 | 23: 3422753 | 96157 | 23: 3441692 | 96158 | 23: 3451376 | 96167 | 23: 3623220 |
| 96169 | 23: 3649638 | 96172 | 23: 3661124 | 96173 | 23: 3691486 | 96174 | 23: 3694848 | 96187 | 23: 3953291 |
| 96195 | 23: 4055037 | 96207 | 23: 4270316 | 96211 | 23: 4397147 | 96257 | 23: 5221777 | 96270 | 23: 5551234 |
| 96273 | 23: 5596536 | 96274 | 23: 5598882 | 96275 | 23: 5598939 | 96276 | 23: 5598954 | 96277 | 23: 5598967 |
| 96291 | 23: 6007499 | 96302 | 23: 6313109 | 96315 | 23: 6596002 | 96316 | 23: 6596059 | 96319 | 23: 6615760 |
| 96320 | 23: 6619709 | 96321 | 23: 6619777 | 96334 | 23: 6834687 | 96343 | 23: 7086052 | 96348 | 23: 7176749 |
| 96349 | 23: 7185030 | 96351 | 23: 7220926 | 96354 | 23: 7287024 | 96362 | 23: 7470856 | 96366 | 23: 7588787 |
| 96367 | 23: 7590703 | 96368 | 23: 7604788 | 96369 | 23: 7627114 | 96370 | 23: 7627927 | 96385 | 23: 8019039 |
| 96386 | 23: 8021311 | 96388 | 23: 8043634 | 96394 | 23: 8257417 | 96395 | 23: 8265499 | 96402 | 23: 8382452 |
| 96403 | 23: 8386569 | 96405 | 23: 8392718 | 96406 | 23: 8394550 | 96407 | 23: 8403739 | 96409 | 23: 8430336 |
| 96410 | 23: 8437470 | 96411 | 23: 8444830 | 96412 | 23: 8458254 | 96423 | 23: 8693541 | 96428 | 23: 8861332 |
| 96429 | 23: 8861347 | 96435 | 23: 8977884 | 96438 | 23: 9024159 | 96439 | 23: 9024206 | 96440 | 23: 9024246 |
| 96447 | 23: 9163290 | 96448 | 23: 9164368 | 96452 | 23: 9341201 | 96457 | 23: 9496576 | 96460 | 23: 9564042 |
| 96461 | 23: 9565542 | 96462 | 23: 9583880 | 96464 | 23: 9685892 | 96466 | 23: 9745811 | 96467 | 23: 9767204 |
| 96476 | 23: 10029783 | 96483 | 23: 10140851 | 96485 | 23: 10256230 | 96486 | 23: 10257763 | 96487 | 23: 10257827 |
| 96488 | 23: 10258971 | 96489 | 23: 10259318 | 96490 | 23: 10259504 | 96492 | 23: 10261010 | 96493 | 23: 10262012 |
| 96494 | 23: 10262085 | 96495 | 23: 10262281 | 96496 | 23: 10262341 | 96497 | 23: 10262386 | 96498 | 23: 10262523 |
| 96501 | 23: 10328877 | 96506 | 23: 10459370 | 96514 | 23: 10615240 | 96515 | 23: 10638380 | 96519 | 23: 10710493 |
| 96523 | 23: 10803832 | 96524 | 23: 10870495 | 96527 | 23: 10883412 | 96528 | 23: 10883649 | 96529 | 23: 10890338 |
| 96530 | 23: 10913209 | 96532 | 23: 10921995 | 96533 | 23: 10922014 | 96538 | 23: 11106125 | 96554 | 23: 11574894 |
| 96555 | 23: 11592749 | 96556 | 23: 11599745 | 96564 | 23: 11737588 | 96566 | 23: 11785803 | 96573 | 23: 11925495 |
| 96575 | 23: 11950761 | 96576 | 23: 11956619 | 96586 | 23: 12073751 | 96591 | 23: 12208427 | 96594 | 23: 12235079 |
| 96603 | 23: 12561054 | 96604 | 23: 12561114 | 96605 | 23: 12561116 | 96606 | 23: 12561252 | 96607 | 23: 12561302 |
| 96608 | 23: 12564591 | 96609 | 23: 12566537 | 96625 | 23: 12975952 | 96626 | 23: 13002407 | 96631 | 23: 13062813 |
| 96632 | 23: 13062870 | 96633 | 23: 13063271 | 96634 | 23: 13065594 | 96635 | 23: 13065660 | 96636 | 23: 13066259 |
| 96637 | 23: 13073019 | 96638 | 23: 13083039 | 96639 | 23: 13096550 | 96640 | 23: 13119734 | 96645 | 23: 13155594 |
| 96650 | 23: 13360736 | 96668 | 23: 13797670 | 96669 | 23: 13803074 | 96673 | 23: 13844075 | 96682 | 23: 13890619 |
| 96683 | 23: 13913723 | 96684 | 23: 13917180 | 96711 | 23: 14503938 | 96713 | 23: 14550205 | 96726 | 23: 14801418 |
| 96736 | 23: 14938004 | 96740 | 23: 14977766 | 96744 | 23: 15085370 | 96746 | 23: 15116016 | 96747 | 23: 15116055 |
| 96748 | 23: 15118596 | 96749 | 23: 15119248 | 96750 | 23: 15119542 | 96751 | 23: 15119571 | 96752 | 23: 15119572 |
| 96753 | 23: 15119619 | 96754 | 23: 15135050 | 96761 | 23: 15271919 | 96763 | 23: 15283396 | 96766 | 23: 15345255 |
| 96773 | 23: 15533652 | 96778 | 23: 15581761 | 96795 | 23: 15759615 | 96801 | 23: 15883997 | 96803 | 23: 15932643 |
| 96813 | 23: 16272106 | 96820 | 23: 16456191 | 96822 | 23: 16466008 | 96823 | 23: 16479564 | 96829 | 23: 16661447 |
| 96831 | 23: 16686014 | 96832 | 23: 16714066 | 96835 | 23: 16792993 | 96836 | 23: 16801536 | 96843 | 23: 17062554 |
| 96859 | 23: 17556957 | 96868 | 23: 17708671 | 96869 | 23: 17718607 | 96870 | 23: 17737639 | 96890 | 23: 18161337 |
| 96891 | 23: 18200714 | 96894 | 23: 18216510 | 96935 | 23: 19331450 | 96936 | 23: 19366522 | 96939 | 23: 19415329 |
| 96966 | 23: 19751742 | 96970 | 23: 19767163 | 96974 | 23: 19826206 | 96976 | 23: 19925315 | 96982 | 23: 19970969 |
| 96985 | 23: 20042631 | 96986 | 23: 20052143 | 96989 | 23: 20102219 | 96991 | 23: 20121033 | 96997 | 23: 20203703 |
| 96998 | 23: 20223241 | 97007 | 23: 20409619 | 97008 | 23: 20409671 | 97009 | 23: 20409761 | 97024 | 23: 20698756 |
| 97025 | 23: 20786968 | 97028 | 23: 20835335 | 97029 | 23: 20835446 | 97045 | 23: 21265221 | 97050 | 23: 21344891 |
| 97056 | 23: 21453135 | 97057 | 23: 21453252 | 97066 | 23: 21870117 | 97085 | 23: 22255326 | 97088 | 23: 22364773 |
| 97113 | 23: 23196063 | 97126 | 23: 23576448 | 97135 | 23: 23755239 | 97136 | 23: 23762207 | 97138 | 23: 23782323 |
| 97148 | 23: 23940335 | 97149 | 23: 23968151 | 97156 | 23: 24092418 | 97164 | 23: 24300414 | 97165 | 23: 24303099 |
| 97167 | 23: 24343369 | 97168 | 23: 24345237 | 97169 | 23: 24352270 | 97173 | 23: 24416257 | 97174 | 23: 24423273 |
| 97179 | 23: 24498401 | 97180 | 23: 24505272 | 97186 | 23: 24683101 | 97187 | 23: 24685752 | 97188 | 23: 24686739 |
| 97189 | 23: 24689461 | 97190 | 23: 24692653 | 97191 | 23: 24699153 | 97192 | 23: 24700095 | 97193 | 23: 24701041 |
| 97194 | 23: 24702797 | 97195 | 23: 24703647 | 97196 | 23: 24704414 | 97197 | 23: 24705785 | 97198 | 23: 24709728 |
| 97200 | 23: 24717517 | 97201 | 23: 24718990 | 97202 | 23: 24723139 | 97203 | 23: 24727842 | 97204 | 23: 24729644 |
| 97205 | 23: 24730615 | 97206 | 23: 24732566 | 97207 | 23: 24733786 | 97208 | 23: 24736986 | 97209 | 23: 24739399 |
| 97210 | 23: 24744157 | 97213 | 23: 24790792 | 97214 | 23: 24794179 | 97216 | 23: 24799157 | 97217 | 23: 24800192 |
| 97218 | 23: 24823698 | 97219 | 23: 24823737 | 97224 | 23: 24880628 | 97226 | 23: 24916092 | 97228 | 23: 24934787 |
| 97229 | 23: 24936283 | 97230 | 23: 24939717 | 97232 | 23: 24975703 | 97240 | 23: 25055772 | 97243 | 23: 25088466 |
| 97244 | 23: 25089919 | 97245 | 23: 25102180 | 97247 | 23: 25129357 | 97248 | 23: 25130486 | 97249 | 23: 25143923 |
| 97250 | 23: 25145004 | 97251 | 23: 25146146 | 97252 | 23: 25156088 | 97253 | 23: 25164433 | 97254 | 23: 25166831 |
| 97257 | 23: 25198505 | 97258 | 23: 25199834 | 97259 | 23: 25200752 | 97260 | 23: 25201563 | 97264 | 23: 25222382 |
| 97265 | 23: 25235072 | 97267 | 23: 25235958 | 97269 | 23: 25250609 | 97271 | 23: 25276948 | 97272 | 23: 25283721 |
| 97273 | 23: 25287389 | 97275 | 23: 25323788 | 97279 | 23: 25354558 | 97280 | 23: 25354656 | 97282 | 23: 25372486 |
| 97286 | 23: 25406424 | 97287 | 23: 25406813 | 97289 | 23: 25433764 | 97292 | 23: 25467868 | 97296 | 23: 25560755 |
| 97298 | 23: 25617278 | 97299 | 23: 25674209 | 97300 | 23: 25680001 | 97301 | 23: 25689132 | 97302 | 23: 25690596 |
| 97303 | 23: 25692139 | 97304 | 23: 25693451 | 97306 | 23: 25715869 | 97308 | 23: 25730240 | 97309 | 23: 25730292 |
| 97310 | 23: 25730339 | 97311 | 23: 25750106 | 97312 | 23: 25755389 | 97313 | 23: 25758772 | 97314 | 23: 25759863 |
| 97315 | 23: 25764182 | 97316 | 23: 25767576 | 97317 | 23: 25780173 | 97318 | 23: 25781822 | 97319 | 23: 25786244 |
| 97320 | 23: 25788400 | 97322 | 23: 25818270 | 97323 | 23: 25819563 | 97324 | 23: 25819612 | 97325 | 23: 25819634 |
| 97327 | 23: 25825333 | 97328 | 23: 25827861 | 97330 | 23: 25834509 | 97331 | 23: 25840572 | 97332 | 23: 25842401 |
| 97335 | 23: 25902950 | 97352 | 23: 26938031 | 97353 | 23: 26943744 | 97354 | 23: 26947613 | 97360 | 23: 27113071 |
| 97361 | 23: 27113077 | 97362 | 23: 27113082 | 97363 | 23: 27113092 | 97364 | 23: 27113093 | 97366 | 23: 27139603 |
| 97367 | 23: 27139619 | 97368 | 23: 27139643 | 97369 | 23: 27141360 | 97370 | 23: 27144341 | 97372 | 23: 27175642 |
| 97373 | 23: 27176260 | 97379 | 23: 27233819 | 97381 | 23: 27306574 | 97382 | 23: 27317678 | 97383 | 23: 27325182 |
| 97384 | 23: 27348931 | 97385 | 23: 27355151 | 97386 | 23: 27356345 | 97387 | 23: 27360333 | 97388 | 23: 27361033 |
| 97389 | 23: 27361247 | 97390 | 23: 27361255 | 97392 | 23: 27385789 | 97393 | 23: 27397427 | 97394 | 23: 27397904 |
| 97395 | 23: 27398878 | 97402 | 23: 27492296 | 97403 | 23: 27493205 | 97404 | 23: 27494111 | 97406 | 23: 27497189 |
| 97407 | 23: 27497222 | 97408 | 23: 27497848 | 97409 | 23: 27497875 | 97410 | 23: 27497880 | 97411 | 23: 27499647 |
| 97412 | 23: 27514878 | 97413 | 23: 27516482 | 97414 | 23: 27522712 | 97415 | 23: 27528967 | 97417 | 23: 27538128 |
| 97419 | 23: 27545924 | 97421 | 23: 27601917 | 97425 | 23: 27635451 | 97426 | 23: 27643343 | 97428 | 23: 27657685 |
| 97431 | 23: 27701786 | 97432 | 23: 27727500 | 97435 | 23: 27774805 | 97436 | 23: 27801348 | 97437 | 23: 27815800 |
| 97438 | 23: 27824468 | 97440 | 23: 27883512 | 97441 | 23: 27910429 | 97442 | 23: 27976925 | 97443 | 23: 27978843 |
| 97445 | 23: 27985061 | 97446 | 23: 27988153 | 97447 | 23: 27988939 | 97449 | 23: 27989644 | 97452 | 23: 28001628 |
| 97453 | 23: 28002243 | 97454 | 23: 28003650 | 97455 | 23: 28006280 | 97456 | 23: 28016941 | 97457 | 23: 28023809 |
| 97458 | 23: 28024215 | 97459 | 23: 28024237 | 97460 | 23: 28029876 | 97461 | 23: 28030196 | 97462 | 23: 28040546 |
| 97463 | 23: 28054764 | 97464 | 23: 28055141 | 97465 | 23: 28055144 | 97466 | 23: 28055147 | 97467 | 23: 28055154 |
| 97468 | 23: 28055203 | 97469 | 23: 28055205 | 97470 | 23: 28056174 | 97471 | 23: 28056245 | 97472 | 23: 28056261 |
| 97473 | 23: 28056279 | 97474 | 23: 28058594 | 97475 | 23: 28084930 | 97476 | 23: 28084959 | 97480 | 23: 28142109 |
| 97481 | 23: 28142880 | 97482 | 23: 28143776 | 97483 | 23: 28147241 | 97485 | 23: 28154077 | 97486 | 23: 28154081 |
| 97487 | 23: 28154567 | 97488 | 23: 28158031 | 97489 | 23: 28162469 | 97490 | 23: 28169587 | 97492 | 23: 28176948 |
| 97493 | 23: 28177862 | 97494 | 23: 28178986 | 97495 | 23: 28180501 | 97496 | 23: 28189617 | 97497 | 23: 28214057 |
| 97499 | 23: 28218286 | 97500 | 23: 28226442 | 97502 | 23: 28247153 | 97504 | 23: 28252270 | 97505 | 23: 28260136 |
| 97506 | 23: 28261564 | 97507 | 23: 28266105 | 97511 | 23: 28289161 | 97512 | 23: 28311837 | 97513 | 23: 28314357 |
| 97514 | 23: 28315369 | 97517 | 23: 28344384 | 97518 | 23: 28345650 | 97520 | 23: 28370592 | 97522 | 23: 28424137 |
| 97523 | 23: 28428619 | 97525 | 23: 28451722 | 97529 | 23: 28527686 | 97530 | 23: 28605105 | 97532 | 23: 28614214 |
| 97533 | 23: 28633036 | 97534 | 23: 28644648 | 97535 | 23: 28667350 | 97536 | 23: 28759020 | 97537 | 23: 28768059 |
| 97538 | 23: 28770375 | 97539 | 23: 28770402 | 97540 | 23: 28770465 | 97541 | 23: 28770499 | 97542 | 23: 28770500 |
| 97543 | 23: 28770527 | 97545 | 23: 28779774 | 97547 | 23: 28784189 | 97549 | 23: 28791635 | 97551 | 23: 28804972 |
| 97552 | 23: 28806779 | 97553 | 23: 28808892 | 97554 | 23: 28811155 | 97555 | 23: 28811333 | 97556 | 23: 28817232 |
| 97557 | 23: 28819161 | 97558 | 23: 28819173 | 97560 | 23: 28820599 | 97561 | 23: 28821000 | 97563 | 23: 28829534 |
| 97564 | 23: 28836068 | 97565 | 23: 28840263 | 97567 | 23: 28855995 | 97568 | 23: 28868586 | 97569 | 23: 28870611 |
| 97570 | 23: 28873879 | 97571 | 23: 28878589 | 97572 | 23: 28881735 | 97574 | 23: 28920628 | 97575 | 23: 28935375 |
| 97576 | 23: 28938250 | 97580 | 23: 28964584 | 97581 | 23: 28967619 | 97582 | 23: 28971358 | 97583 | 23: 28980855 |
| 97584 | 23: 29001677 | 97585 | 23: 29002896 | 97586 | 23: 29003973 | 97587 | 23: 29004861 | 97589 | 23: 29028277 |
| 97591 | 23: 29088900 | 97593 | 23: 29127784 | 97595 | 23: 29155100 | 97598 | 23: 29200199 | 97599 | 23: 29231778 |
| 97604 | 23: 29351343 | 97605 | 23: 29351673 | 97606 | 23: 29387809 | 97608 | 23: 29405131 | 97610 | 23: 29505622 |
| 97618 | 23: 29739156 | 97619 | 23: 29739161 | 97629 | 23: 30015425 | 97633 | 23: 30085624 | 97641 | 23: 30343016 |
| 97643 | 23: 30357104 | 97644 | 23: 30377231 | 97645 | 23: 30377501 | 97646 | 23: 30391263 | 97647 | 23: 30408989 |
| 97648 | 23: 30411874 | 97649 | 23: 30417585 | 97650 | 23: 30422451 | 97651 | 23: 30442710 | 97652 | 23: 30443193 |
| 97653 | 23: 30447941 | 97654 | 23: 30464791 | 97656 | 23: 30478896 | 97660 | 23: 30538186 | 97662 | 23: 30568716 |
| 97669 | 23: 30789137 | 97670 | 23: 30789578 | 97671 | 23: 30789711 | 97685 | 23: 31163020 | 97687 | 23: 31193034 |
| 97688 | 23: 31201200 | 97689 | 23: 31207608 | 97690 | 23: 31237063 | 97691 | 23: 31239018 | 97696 | 23: 31408344 |
| 97697 | 23: 31474420 | 97698 | 23: 31478356 | 97699 | 23: 31478871 | 97706 | 23: 31666638 | 97708 | 23: 31712475 |
| 97709 | 23: 31713030 | 97710 | 23: 31713883 | 97712 | 23: 31795823 | 97716 | 23: 31839586 | 97717 | 23: 31847799 |
| 97718 | 23: 31860518 | 97719 | 23: 31864204 | 97720 | 23: 31883086 | 97721 | 23: 31885522 | 97723 | 23: 31892464 |
| 97724 | 23: 31898107 | 97731 | 23: 32103028 | 97735 | 23: 32155949 | 97748 | 23: 32348823 | 97754 | 23: 32429180 |
| 97755 | 23: 32429299 | 97756 | 23: 32429437 | 97760 | 23: 32498379 | 97763 | 23: 32587067 | 97769 | 23: 32714057 |
| 97776 | 23: 32953339 | 97777 | 23: 32953359 | 97778 | 23: 32954768 | 97779 | 23: 32963026 | 97781 | 23: 32991047 |
| 97782 | 23: 33010094 | 97783 | 23: 33010248 | 97784 | 23: 33017162 | 97793 | 23: 33209372 | 97796 | 23: 33232305 |
| 97809 | 23: 33564304 | 97823 | 23: 33923637 | 97829 | 23: 34147241 | 97832 | 23: 34255293 | 97846 | 23: 34559019 |
| 97847 | 23: 34568421 | 97872 | 23: 35579146 | 97874 | 23: 35643122 | 97882 | 23: 35853906 | 97884 | 23: 35933978 |
| 97897 | 23: 36340613 | 97903 | 23: 36491282 | 97904 | 23: 36522078 | 97911 | 23: 36715268 | 97922 | 23: 37003285 |
| 97923 | 23: 37008791 | 97926 | 23: 37147881 | 97929 | 23: 37226966 | 97931 | 23: 37255744 | 97933 | 23: 37290169 |
| 97934 | 23: 37311602 | 97938 | 23: 37438508 | 97939 | 23: 37509546 | 97948 | 23: 37644241 | 97952 | 23: 37725066 |
| 97954 | 23: 37743052 | 97964 | 23: 38103066 | 97969 | 23: 38331199 | 97970 | 23: 38380507 | 97973 | 23: 38417336 |
| 97995 | 23: 38995562 | 98005 | 23: 39248806 | 98007 | 23: 39300011 | 98009 | 23: 39319903 | 98029 | 23: 39426076 |
| 98034 | 23: 39454222 | 98046 | 23: 39542549 | 98058 | 23: 39582084 | 98065 | 23: 39599289 | 98072 | 23: 39623727 |
| 98074 | 23: 39636483 | 98076 | 23: 39640935 | 98083 | 23: 39789176 | 98085 | 23: 39815156 | 98090 | 23: 39928196 |
| 98093 | 23: 39972622 | 98097 | 23: 40044291 | 98098 | 23: 40064417 | 98106 | 23: 40177352 | 98107 | 23: 40190398 |
| 98109 | 23: 40223350 | 98111 | 23: 40255790 | 98112 | 23: 40270530 | 98116 | 23: 40376606 | 98119 | 23: 40424669 |
| 98121 | 23: 40430850 | 98123 | 23: 40480107 | 98124 | 23: 40500673 | 98125 | 23: 40564407 | 98131 | 23: 40662422 |
| 98133 | 23: 40688993 | 98136 | 23: 40758273 | 98139 | 23: 40791971 | 98156 | 23: 41037376 | 98161 | 23: 41085635 |
| 98163 | 23: 41110764 | 98177 | 23: 41370057 | 98178 | 23: 41371667 | 98179 | 23: 41393226 | 98182 | 23: 41488055 |
| 98198 | 23: 41873620 | 98228 | 23: 42474522 | 98251 | 23: 43050343 | 98257 | 23: 43142746 | 98266 | 23: 43402438 |
| 98282 | 23: 43699449 | 98288 | 23: 43890903 | 98290 | 23: 43922804 | 98325 | 23: 44517863 | 98326 | 23: 44517941 |
| 98332 | 23: 44610396 | 98341 | 23: 44856499 | 98343 | 23: 44879028 | 98344 | 23: 44896794 | 98345 | 23: 44914975 |
| 98346 | 23: 44944349 | 98354 | 23: 45035699 | 98357 | 23: 45057099 | 98358 | 23: 45063024 | 98378 | 23: 45357440 |
| 98380 | 23: 45397415 | 98381 | 23: 45398681 | 98383 | 23: 45430124 | 98384 | 23: 45432927 | 98390 | 23: 45560731 |
| 98404 | 23: 46021030 | 98405 | 23: 46022790 | 98406 | 23: 46022913 | 98407 | 23: 46023921 | 98408 | 23: 46024140 |
| 98419 | 23: 46342707 | 98424 | 23: 46476087 | 98434 | 23: 46728089 | 98439 | 23: 46821712 | 98440 | 23: 46867745 |
| 98445 | 23: 46932972 | 98447 | 23: 46982531 | 98448 | 23: 46982538 | 98449 | 23: 46982544 | 98450 | 23: 46982553 |
| 98451 | 23: 46982569 | 98452 | 23: 46982672 | 98453 | 23: 46982688 | 98469 | 23: 47346112 | 98470 | 23: 47346172 |
| 98472 | 23: 47377820 | 98476 | 23: 47501209 | 98479 | 23: 47512112 | 98480 | 23: 47535388 | 98493 | 23: 47816262 |
| 98494 | 23: 47825640 | 98495 | 23: 47825678 | 98496 | 23: 47826029 | 98497 | 23: 47826560 | 98498 | 23: 47827373 |
| 98500 | 23: 47839983 | 98501 | 23: 47840868 | 98503 | 23: 47845586 | 98504 | 23: 47847036 | 98523 | 23: 48330566 |
| 98529 | 23: 48410101 | 98533 | 23: 48581773 | 98538 | 23: 48634859 | 98545 | 23: 48734960 | 98547 | 23: 48752878 |
| 98555 | 23: 48901109 | 98567 | 23: 49079717 | 98569 | 23: 49107918 | 98574 | 23: 49198514 | 98576 | 23: 49210048 |
| 98584 | 23: 49312924 | 98585 | 23: 49312984 | 98586 | 23: 49312985 | 98587 | 23: 49312986 | 98592 | 23: 49424994 |
| 98593 | 23: 49432035 | 98594 | 23: 49442339 | 98602 | 23: 49590910 | 98622 | 23: 49941067 | 98624 | 23: 49942903 |
| 98625 | 23: 49942904 | 98632 | 23: 50043273 | 98635 | 23: 50078664 | 98636 | 23: 50114133 | 98638 | 23: 50169252 |
| 98642 | 23: 50258011 | 98649 | 23: 50375296 | 98664 | 23: 50638453 | 98671 | 23: 50875493 | 98678 | 23: 50932969 |
| 98680 | 23: 50964263 | 98686 | 23: 51018373 | 98689 | 23: 51079827 | 98691 | 23: 51116781 | 98701 | 23: 51298091 |
| 98711 | 23: 51501609 | 98718 | 23: 51674082 | 98722 | 23: 51744554 | 98728 | 23: 51847760 | 98737 | 23: 52008498 |
| 98741 | 23: 52053452 | 98743 | 23: 52076716 | 98781 | 24: 469885 | 98798 | 24: 880857 | 98799 | 24: 888643 |
| 98802 | 24: 930959 | 98805 | 24: 980184 | 98808 | 24: 1046300 | 98818 | 24: 1170762 | 98819 | 24: 1171661 |
| 98834 | 24: 1570237 | 98851 | 24: 1855510 | 98862 | 24: 2006568 | 98871 | 24: 2265624 | 98872 | 24: 2276656 |
| 98900 | 24: 2945776 | 98902 | 24: 2978064 | 98918 | 24: 3289918 | 98930 | 24: 3538543 | 98933 | 24: 3696514 |
| 98940 | 24: 3908092 | 98942 | 24: 3966055 | 98943 | 24: 3966359 | 98947 | 24: 4007654 | 98948 | 24: 4007678 |
| 98949 | 24: 4012661 | 98950 | 24: 4013403 | 98951 | 24: 4021260 | 98956 | 24: 4164775 | 98957 | 24: 4164916 |
| 98958 | 24: 4164934 | 98959 | 24: 4165918 | 98960 | 24: 4168088 | 98965 | 24: 4188159 | 98970 | 24: 4291963 |
| 98974 | 24: 4399899 | 98980 | 24: 4581542 | 98982 | 24: 4612976 | 98989 | 24: 4963101 | 98995 | 24: 5087606 |
| 99004 | 24: 5305296 | 99005 | 24: 5333407 | 99006 | 24: 5337650 | 99018 | 24: 5642653 | 99026 | 24: 5818978 |
| 99038 | 24: 6264925 | 99076 | 24: 7162546 | 99078 | 24: 7286305 | 99086 | 24: 7467215 | 99111 | 24: 8069004 |
| 99112 | 24: 8108977 | 99113 | 24: 8109036 | 99116 | 24: 8121802 | 99118 | 24: 8175362 | 99119 | 24: 8182146 |
| 99146 | 24: 9005035 | 99151 | 24: 9085950 | 99190 | 24: 9913417 | 99192 | 24: 9977557 | 99276 | 24: 10203460 |
| 99283 | 24: 10336705 | 99286 | 24: 10405660 | 99292 | 24: 10590068 | 99293 | 24: 10590091 | 99311 | 24: 11064065 |
| 99321 | 24: 11243732 | 99322 | 24: 11285722 | 99329 | 24: 11451899 | 99339 | 24: 11738379 | 99359 | 24: 12247620 |
| 99364 | 24: 12466661 | 99365 | 24: 12471212 | 99371 | 24: 12650426 | 99372 | 24: 12652362 | 99373 | 24: 12660326 |
| 99396 | 24: 13228420 | 99405 | 24: 13465765 | 99418 | 24: 13789354 | 99419 | 24: 13790831 | 99420 | 24: 13791730 |
| 99427 | 24: 14119618 | 99459 | 24: 14988931 | 99484 | 24: 15813747 | 99490 | 24: 16186692 | 99501 | 24: 16499172 |
| 99503 | 24: 16506987 | 99509 | 24: 16666243 | 99528 | 24: 17162739 | 99530 | 24: 17208682 | 99543 | 24: 17625014 |
| 99565 | 24: 18353069 | 99574 | 24: 18678355 | 99598 | 24: 19285724 | 99603 | 24: 19437043 | 99607 | 24: 19606961 |
| 99610 | 24: 19713861 | 99615 | 24: 19770957 | 99628 | 24: 20349623 | 99629 | 24: 20358408 | 99632 | 24: 20393549 |
| 99638 | 24: 20499578 | 99639 | 24: 20510429 | 99640 | 24: 20510450 | 99641 | 24: 20510487 | 99642 | 24: 20510488 |
| 99643 | 24: 20510505 | 99644 | 24: 20510579 | 99645 | 24: 20510583 | 99646 | 24: 20510587 | 99647 | 24: 20510597 |
| 99648 | 24: 20510600 | 99649 | 24: 20513392 | 99650 | 24: 20513424 | 99651 | 24: 20513517 | 99652 | 24: 20521127 |
| 99653 | 24: 20532438 | 99654 | 24: 20532441 | 99655 | 24: 20532446 | 99656 | 24: 20532502 | 99657 | 24: 20572686 |
| 99669 | 24: 20847626 | 99671 | 24: 20874971 | 99672 | 24: 20920443 | 99673 | 24: 20920491 | 99674 | 24: 20920727 |
| 99675 | 24: 20922777 | 99676 | 24: 20926914 | 99677 | 24: 20927000 | 99678 | 24: 20927024 | 99679 | 24: 20927059 |
| 99680 | 24: 20929398 | 99681 | 24: 20929449 | 99682 | 24: 20929473 | 99693 | 24: 21272688 | 99695 | 24: 21306399 |
| 99696 | 24: 21375203 | 99697 | 24: 21376904 | 99698 | 24: 21376940 | 99699 | 24: 21376967 | 99700 | 24: 21376972 |
| 99701 | 24: 21378281 | 99702 | 24: 21378321 | 99703 | 24: 21378352 | 99704 | 24: 21378382 | 99705 | 24: 21378383 |
| 99706 | 24: 21378384 | 99708 | 24: 21378440 | 99709 | 24: 21378445 | 99711 | 24: 21395177 | 99712 | 24: 21396591 |
| 99716 | 24: 21432058 | 99717 | 24: 21452284 | 99718 | 24: 21456850 | 99719 | 24: 21471419 | 99723 | 24: 21488223 |
| 99725 | 24: 21504166 | 99729 | 24: 21560026 | 99730 | 24: 21560060 | 99732 | 24: 21564234 | 99733 | 24: 21587057 |
| 99734 | 24: 21590358 | 99735 | 24: 21595564 | 99736 | 24: 21598260 | 99737 | 24: 21605659 | 99738 | 24: 21620362 |
| 99739 | 24: 21667119 | 99740 | 24: 21721258 | 99742 | 24: 21765426 | 99754 | 24: 21940194 | 99756 | 24: 21971534 |
| 99762 | 24: 22139528 | 99769 | 24: 22384770 | 99777 | 24: 22507325 | 99779 | 24: 22552252 | 99786 | 24: 22901733 |
| 99787 | 24: 22901743 | 99788 | 24: 22901792 | 99789 | 24: 22901810 | 99790 | 24: 22901819 | 99795 | 24: 23015622 |
| 99797 | 24: 23048740 | 99798 | 24: 23048839 | 99800 | 24: 23138496 | 99808 | 24: 23304844 | 99809 | 24: 23357754 |
| 99812 | 24: 23476124 | 99840 | 24: 24470786 | 99848 | 24: 24601724 | 99850 | 24: 24651791 | 99856 | 24: 24861946 |
| 99858 | 24: 24942299 | 99859 | 24: 24968711 | 99864 | 24: 25055928 | 99867 | 24: 25166673 | 99870 | 24: 25259209 |
| 99871 | 24: 25291130 | 99875 | 24: 25393251 | 99880 | 24: 25549639 | 99881 | 24: 25550670 | 99882 | 24: 25554087 |
| 99883 | 24: 25555252 | 99884 | 24: 25586246 | 99887 | 24: 25655955 | 99891 | 24: 25728080 | 99892 | 24: 25730700 |
| 99893 | 24: 25732798 | 99895 | 24: 25765374 | 99901 | 24: 25840462 | 99928 | 24: 26414878 | 99929 | 24: 26414942 |
| 99939 | 24: 26752400 | 99960 | 24: 27335211 | 99976 | 24: 27659643 | 99977 | 24: 27671338 | 99978 | 24: 27707920 |
| 99987 | 24: 27976475 | 99992 | 24: 28073538 | 10001 | 24: 28717465 | 10002 | 24: 28990565 | 10002 | 24: 28990603 |
| 10002 | 24: 28990620 | 10003 | 24: 29071264 | 10003 | 24: 29205694 | 10003 | 24: 29294106 | 10004 | 24: 29309403 |
| 10005 | 24: 29593728 | 10005 | 24: 29785127 | 10006 | 24: 30022971 | 10006 | 24: 30028664 | 10006 | 24: 30028736 |
| 10007 | 24: 30132653 | 10008 | 24: 30418113 | 10010 | 24: 30736100 | 10010 | 24: 30756120 | 10010 | 24: 30756121 |
| 10011 | 24: 31302891 | 10011 | 24: 31387621 | 10012 | 24: 31602471 | 10014 | 24: 31910298 | 10015 | 24: 32159647 |
| 10015 | 24: 32159730 | 10016 | 24: 32335210 | 10017 | 24: 32561115 | 10017 | 24: 32732872 | 10017 | 24: 32732892 |
| 10018 | 24: 32732952 | 10018 | 24: 32735243 | 10018 | 24: 32769877 | 10018 | 24: 32810095 | 10018 | 24: 32824750 |
| 10018 | 24: 32826968 | 10019 | 24: 32827828 | 10019 | 24: 32830047 | 10019 | 24: 32830055 | 10019 | 24: 32840723 |
| 10019 | 24: 32857855 | 10019 | 24: 32868393 | 10019 | 24: 32875942 | 10020 | 24: 32894247 | 10020 | 24: 33166170 |
| 10021 | 24: 33227163 | 10021 | 24: 33227191 | 10023 | 24: 33780521 | 10025 | 24: 34326602 | 10025 | 24: 34364918 |
| 10026 | 24: 34614458 | 10026 | 24: 34760712 | 10026 | 24: 34762120 | 10027 | 24: 34813345 | 10028 | 24: 35185894 |
| 10028 | 24: 35185970 | 10029 | 24: 35309422 | 10029 | 24: 35335089 | 10029 | 24: 35472794 | 10032 | 24: 35837548 |
| 10034 | 24: 36589347 | 10034 | 24: 36589477 | 10035 | 24: 36835198 | 10035 | 24: 36988061 | 10036 | 24: 37134995 |
| 10036 | 24: 37239650 | 10036 | 24: 37280274 | 10036 | 24: 37304503 | 10036 | 24: 37306947 | 10037 | 24: 37417740 |
| 10038 | 24: 37592997 | 10038 | 24: 37706524 | 10039 | 24: 37931336 | 10040 | 24: 38475906 | 10040 | 24: 38609603 |
| 10041 | 24: 38809089 | 10042 | 24: 38949417 | 10043 | 24: 39200562 | 10043 | 24: 39308113 | 10043 | 24: 39456209 |
| 10043 | 24: 39464085 | 10044 | 24: 39492963 | 10044 | 24: 39687343 | 10045 | 24: 39707191 | 10045 | 24: 39897863 |
| 10045 | 24: 39902640 | 10046 | 24: 39906046 | 10046 | 24: 39906190 | 10046 | 24: 39913847 | 10047 | 24: 40288339 |
| 10049 | 24: 40342848 | 10049 | 24: 40352389 | 10049 | 24: 40395247 | 10049 | 24: 40408675 | 10050 | 24: 40466974 |
| 10050 | 24: 40545027 | 10050 | 24: 40573682 | 10051 | 24: 40583210 | 10051 | 24: 40583246 | 10051 | 24: 40587358 |
| 10051 | 24: 40746951 | 10052 | 24: 40756834 | 10052 | 24: 40764626 | 10052 | 24: 40781770 | 10052 | 24: 40798538 |
| 10052 | 24: 40803087 | 10053 | 24: 40974776 | 10054 | 24: 41093545 | 10054 | 24: 41180993 | 10054 | 24: 41204807 |
| 10056 | 24: 41805610 | 10057 | 24: 41935257 | 10057 | 24: 41944725 | 10057 | 24: 42049855 | 10058 | 24: 42141779 |
| 10058 | 24: 42231198 | 10058 | 24: 42251623 | 10058 | 24: 42272285 | 10058 | 24: 42276093 | 10059 | 24: 42324465 |
| 10059 | 24: 42659053 | 10060 | 24: 42659123 | 10060 | 24: 42659180 | 10060 | 24: 42659202 | 10060 | 24: 42709614 |
| 10060 | 24: 42721124 | 10060 | 24: 42724162 | 10060 | 24: 42725838 | 10060 | 24: 42726075 | 10060 | 24: 42727665 |
| 10061 | 24: 42768635 | 10061 | 24: 42779119 | 10061 | 24: 42816514 | 10061 | 24: 42881112 | 10063 | 24: 43435997 |
| 10063 | 24: 43500003 | 10064 | 24: 44069431 | 10065 | 24: 44298375 | 10066 | 24: 44375642 | 10066 | 24: 44396418 |
| 10066 | 24: 44471971 | 10067 | 24: 44658910 | 10067 | 24: 44735016 | 10068 | 24: 44775120 | 10068 | 24: 44792435 |
| 10069 | 24: 45080627 | 10070 | 24: 45199003 | 10070 | 24: 45243583 | 10071 | 24: 45357351 | 10071 | 24: 45461858 |
| 10071 | 24: 45489006 | 10071 | 24: 45499596 | 10072 | 24: 45501942 | 10072 | 24: 45537440 | 10072 | 24: 45683209 |
| 10073 | 24: 45738887 | 10073 | 24: 45745474 | 10073 | 24: 45809276 | 10073 | 24: 45824753 | 10074 | 24: 45922636 |
| 10075 | 24: 45923194 | 10075 | 24: 46005539 | 10077 | 24: 46107365 | 10077 | 24: 46128800 | 10077 | 24: 46131144 |
| 10077 | 24: 46200299 | 10078 | 24: 46293419 | 10078 | 24: 46339304 | 10078 | 24: 46339449 | 10078 | 24: 46343493 |
| 10078 | 24: 46349257 | 10079 | 24: 46658398 | 10080 | 24: 46735235 | 10080 | 24: 46748616 | 10080 | 24: 46860941 |
| 10080 | 24: 46863126 | 10080 | 24: 46872067 | 10081 | 24: 46872779 | 10081 | 24: 46872783 | 10081 | 24: 46872835 |
| 10081 | 24: 46874235 | 10082 | 24: 47186952 | 10083 | 24: 47493053 | 10083 | 24: 47504673 | 10083 | 24: 47575681 |
| 10084 | 24: 47792768 | 10084 | 24: 47861526 | 10085 | 24: 47942540 | 10086 | 24: 48132259 | 10086 | 24: 48283076 |
| 10087 | 24: 48398019 | 10087 | 24: 48400794 | 10088 | 24: 48949533 | 10089 | 24: 49153895 | 10089 | 24: 49213874 |
| 10089 | 24: 49220538 | 10090 | 24: 49271697 | 10090 | 24: 49386058 | 10090 | 24: 49461650 | 10091 | 24: 49808792 |
| 10092 | 24: 49949844 | 10092 | 24: 49951357 | 10092 | 24: 49954724 | 10093 | 24: 50278508 | 10093 | 24: 50323489 |
| 10093 | 24: 50330708 | 10094 | 24: 50485176 | 10095 | 24: 50664362 | 10095 | 24: 50886055 | 10096 | 24: 51077466 |
| 10096 | 24: 51079480 | 10098 | 24: 51488836 | 10101 | 24: 51956661 | 10101 | 24: 52119070 | 10102 | 24: 52251430 |
| 10102 | 24: 52257462 | 10102 | 24: 52257497 | 10104 | 24: 52725727 | 10105 | 24: 53039441 | 10105 | 24: 53060437 |
| 10106 | 24: 53471017 | 10107 | 24: 53476039 | 10107 | 24: 53480782 | 10107 | 24: 53485369 | 10107 | 24: 53489339 |
| 10108 | 24: 53695135 | 10109 | 24: 54141567 | 10110 | 24: 54171685 | 10110 | 24: 54227463 | 10111 | 24: 54361200 |
| 10111 | 24: 54366075 | 10112 | 24: 54596604 | 10112 | 24: 54605333 | 10113 | 24: 54678133 | 10113 | 24: 54772296 |
| 10114 | 24: 54837850 | 10114 | 24: 54844868 | 10114 | 24: 54868156 | 10116 | 24: 55295472 | 10116 | 24: 55304369 |
| 10117 | 24: 55700714 | 10118 | 24: 55732464 | 10118 | 24: 55736959 | 10118 | 24: 55739826 | 10118 | 24: 55923426 |
| 10119 | 24: 56024340 | 10119 | 24: 56024373 | 10119 | 24: 56024406 | 10119 | 24: 56098690 | 10119 | 24: 56102284 |
| 10119 | 24: 56102647 | 10120 | 24: 56102653 | 10120 | 24: 56102851 | 10122 | 24: 56688810 | 10122 | 24: 56746296 |
| 10122 | 24: 56746319 | 10123 | 24: 56797648 | 10123 | 24: 56813204 | 10125 | 24: 57240950 | 10125 | 24: 57371376 |
| 10125 | 24: 57399764 | 10126 | 24: 57412925 | 10126 | 24: 57552725 | 10127 | 24: 57558999 | 10127 | 24: 57626126 |
| 10128 | 24: 57747906 | 10128 | 24: 57797863 | 10129 | 24: 58058760 | 10129 | 24: 58124009 | 10131 | 24: 58425089 |
| 10132 | 24: 58468125 | 10132 | 24: 58577110 | 10132 | 24: 58601846 | 10134 | 24: 58910690 | 10135 | 24: 59164671 |
| 10135 | 24: 59196985 | 10136 | 24: 59421526 | 10136 | 24: 59468734 | 10138 | 24: 59829538 | 10142 | 24: 60586093 |
| 10142 | 24: 60623865 | 10142 | 24: 60632706 | 10144 | 24: 60989011 | 10144 | 24: 61029712 | 10144 | 24: 61052179 |
| 10144 | 24: 61119884 | 10145 | 24: 61260912 | 10146 | 24: 61463290 | 10146 | 24: 61466635 | 10146 | 24: 61477326 |
| 10146 | 24: 61528992 | 10147 | 24: 61571637 | 10147 | 24: 61692816 | 10148 | 24: 61746382 | 10150 | 25: 121555 |
| 10150 | 25: 127561 | 10151 | 25: 343439 | 10151 | 25: 373190 | 10152 | 25: 376453 | 10152 | 25: 424297 |
| 10152 | 25: 567175 | 10152 | 25: 572006 | 10152 | 25: 670011 | 10152 | 25: 670169 | 10154 | 25: 950550 |
| 10154 | 25: 1045088 | 10154 | 25: 1087084 | 10154 | 25: 1134920 | 10155 | 25: 1139788 | 10155 | 25: 1177714 |
| 10155 | 25: 1296800 | 10155 | 25: 1324226 | 10156 | 25: 1352107 | 10156 | 25: 1353932 | 10156 | 25: 1354851 |
| 10156 | 25: 1364554 | 10156 | 25: 1364583 | 10156 | 25: 1365638 | 10157 | 25: 1405987 | 10157 | 25: 1476171 |
| 10157 | 25: 1478625 | 10158 | 25: 1649137 | 10158 | 25: 1775261 | 10158 | 25: 1777637 | 10159 | 25: 1777709 |
| 10159 | 25: 1777710 | 10159 | 25: 1777721 | 10159 | 25: 1783080 | 10159 | 25: 1790972 | 10159 | 25: 1791028 |
| 10159 | 25: 1808653 | 10159 | 25: 1818249 | 10160 | 25: 1835147 | 10160 | 25: 1862652 | 10160 | 25: 1883004 |
| 10160 | 25: 1966690 | 10161 | 25: 2003071 | 10162 | 25: 2200434 | 10162 | 25: 2247195 | 10165 | 25: 2376504 |
| 10166 | 25: 2422717 | 10166 | 25: 2477784 | 10166 | 25: 2479395 | 10166 | 25: 2479688 | 10166 | 25: 2479754 |
| 10167 | 25: 2537288 | 10167 | 25: 2538507 | 10167 | 25: 2540535 | 10167 | 25: 2547137 | 10167 | 25: 2548630 |
| 10167 | 25: 2552738 | 10167 | 25: 2553655 | 10167 | 25: 2555768 | 10167 | 25: 2560887 | 10167 | 25: 2561123 |
| 10168 | 25: 2563116 | 10168 | 25: 2564127 | 10168 | 25: 2565592 | 10168 | 25: 2568326 | 10168 | 25: 2573673 |
| 10168 | 25: 2575681 | 10168 | 25: 2578879 | 10168 | 25: 2580437 | 10168 | 25: 2581459 | 10169 | 25: 2582592 |
| 10169 | 25: 2585032 | 10169 | 25: 2586441 | 10170 | 25: 2681110 | 10170 | 25: 2681129 | 10170 | 25: 2687754 |
| 10170 | 25: 2695684 | 10170 | 25: 2695701 | 10170 | 25: 2695795 | 10170 | 25: 2695845 | 10171 | 25: 2695937 |
| 10171 | 25: 2696055 | 10171 | 25: 2696181 | 10171 | 25: 2696410 | 10171 | 25: 2698504 | 10171 | 25: 2699035 |
| 10171 | 25: 2699105 | 10171 | 25: 2739170 | 10171 | 25: 2740282 | 10171 | 25: 2740291 | 10172 | 25: 2743006 |
| 10172 | 25: 2743075 | 10172 | 25: 2743261 | 10172 | 25: 2743406 | 10172 | 25: 2749450 | 10172 | 25: 2762252 |
| 10172 | 25: 2762309 | 10172 | 25: 2775821 | 10173 | 25: 2819065 | 10173 | 25: 2819396 | 10173 | 25: 2819471 |
| 10173 | 25: 2819522 | 10173 | 25: 2819528 | 10173 | 25: 2819546 | 10173 | 25: 2819603 | 10175 | 25: 3210540 |
| 10175 | 25: 3310916 | 10176 | 25: 3363490 | 10176 | 25: 3426286 | 10176 | 25: 3477152 | 10177 | 25: 3524368 |
| 10177 | 25: 3529015 | 10177 | 25: 3534470 | 10177 | 25: 3534510 | 10177 | 25: 3554022 | 10178 | 25: 3581070 |
| 10178 | 25: 3588116 | 10178 | 25: 3595733 | 10178 | 25: 3604536 | 10178 | 25: 3606474 | 10178 | 25: 3625688 |
| 10178 | 25: 3630953 | 10178 | 25: 3640402 | 10178 | 25: 3650316 | 10178 | 25: 3656324 | 10179 | 25: 3701510 |
| 10179 | 25: 3830536 | 10179 | 25: 3851824 | 10180 | 25: 3903874 | 10180 | 25: 4088166 | 10181 | 25: 4278985 |
| 10181 | 25: 4319018 | 10185 | 25: 5024647 | 10186 | 25: 5186113 | 10186 | 25: 5326044 | 10191 | 25: 5967822 |
| 10192 | 25: 6012533 | 10193 | 25: 6175707 | 10193 | 25: 6231384 | 10195 | 25: 6788634 | 10196 | 25: 6838881 |
| 10196 | 25: 6862509 | 10196 | 25: 6862547 | 10198 | 25: 7313080 | 10198 | 25: 7484309 | 10198 | 25: 7517426 |
| 10199 | 25: 7734903 | 10199 | 25: 7840771 | 10199 | 25: 7840811 | 10200 | 25: 7884771 | 10200 | 25: 7905114 |
| 10201 | 25: 7941201 | 10201 | 25: 8064816 | 10202 | 25: 8388133 | 10202 | 25: 8511991 | 10204 | 25: 8788906 |
| 10204 | 25: 8844118 | 10205 | 25: 9197230 | 10206 | 25: 9300591 | 10206 | 25: 9365404 | 10206 | 25: 9378741 |
| 10206 | 25: 9385962 | 10207 | 25: 9502049 | 10207 | 25: 9643363 | 10207 | 25: 9687538 | 10208 | 25: 9776752 |
| 10208 | 25: 9843435 | 10209 | 25: 9993793 | 10209 | 25: 10007062 | 10210 | 25: 10139839 | 10210 | 25: 10198288 |
| 10210 | 25: 10227883 | 10210 | 25: 10272497 | 10211 | 25: 10365727 | 10211 | 25: 10541563 | 10211 | 25: 10541576 |
| 10212 | 25: 10547353 | 10213 | 25: 10832103 | 10213 | 25: 10836311 | 10213 | 25: 10948241 | 10213 | 25: 10951982 |
| 10214 | 25: 11012348 | 10214 | 25: 11110219 | 10214 | 25: 11111271 | 10215 | 25: 11138838 | 10216 | 25: 11274257 |
| 10216 | 25: 11436742 | 10216 | 25: 11437820 | 10217 | 25: 11447709 | 10217 | 25: 11455980 | 10217 | 25: 11549424 |
| 10217 | 25: 11549497 | 10217 | 25: 11570419 | 10220 | 25: 12214562 | 10221 | 25: 12401343 | 10223 | 25: 12943128 |
| 10224 | 25: 13251854 | 10224 | 25: 13339029 | 10224 | 25: 13345472 | 10225 | 25: 13388375 | 10226 | 25: 13601294 |
| 10227 | 25: 14013151 | 10228 | 25: 14073120 | 10228 | 25: 14201547 | 10229 | 25: 14377407 | 10229 | 25: 14409676 |
| 10229 | 25: 14448614 | 10229 | 25: 14511967 | 10230 | 25: 14519922 | 10230 | 25: 14583409 | 10231 | 25: 14703237 |
| 10231 | 25: 14862354 | 10232 | 25: 15092643 | 10233 | 25: 15276545 | 10233 | 25: 15276571 | 10233 | 25: 15370203 |
| 10235 | 25: 15564906 | 10235 | 25: 15633665 | 10235 | 25: 15633709 | 10235 | 25: 15633785 | 10236 | 25: 15912161 |
| 10236 | 25: 15942929 | 10236 | 25: 15943009 | 10237 | 25: 15996288 | 10237 | 25: 16093653 | 10238 | 25: 16219145 |
| 10238 | 25: 16288361 | 10238 | 25: 16288367 | 10239 | 25: 16429229 | 10240 | 25: 16494465 | 10242 | 25: 17127180 |
| 10243 | 25: 17439767 | 10243 | 25: 17446840 | 10244 | 25: 17474953 | 10244 | 25: 17475010 | 10244 | 25: 17483539 |
| 10244 | 25: 17488467 | 10245 | 25: 17838540 | 10246 | 25: 18019077 | 10246 | 25: 18091054 | 10247 | 25: 18283899 |
| 10247 | 25: 18285948 | 10248 | 25: 18730651 | 10248 | 25: 18747611 | 10248 | 25: 18760366 | 10248 | 25: 18798985 |
| 10249 | 25: 18976796 | 10250 | 25: 18988158 | 10253 | 25: 19881913 | 10253 | 25: 19929634 | 10254 | 25: 20012375 |
| 10254 | 25: 20043037 | 10254 | 25: 20085662 | 10255 | 25: 20338297 | 10257 | 25: 21049790 | 10257 | 25: 21139359 |
| 10258 | 25: 21196203 | 10258 | 25: 21260648 | 10258 | 25: 21317697 | 10259 | 25: 21318468 | 10259 | 25: 21327438 |
| 10259 | 25: 21329507 | 10259 | 25: 21405459 | 10260 | 25: 21506832 | 10260 | 25: 21524798 | 10260 | 25: 21532563 |
| 10262 | 25: 22103985 | 10264 | 25: 22376703 | 10264 | 25: 22394285 | 10264 | 25: 22420195 | 10265 | 25: 22670949 |
| 10268 | 25: 23399437 | 10269 | 25: 23750537 | 10271 | 25: 24033949 | 10271 | 25: 24132273 | 10272 | 25: 24242643 |
| 10272 | 25: 24361949 | 10274 | 25: 24619856 | 10274 | 25: 24768804 | 10275 | 25: 24829402 | 10275 | 25: 24857183 |
| 10275 | 25: 24902475 | 10275 | 25: 24915980 | 10275 | 25: 24918063 | 10275 | 25: 24918979 | 10276 | 25: 24918980 |
| 10276 | 25: 24920501 | 10277 | 25: 25159107 | 10277 | 25: 25226497 | 10278 | 25: 25231198 | 10278 | 25: 25252790 |
| 10278 | 25: 25319545 | 10278 | 25: 25319598 | 10278 | 25: 25327412 | 10278 | 25: 25327437 | 10278 | 25: 25355320 |
| 10278 | 25: 25355323 | 10279 | 25: 25370729 | 10280 | 25: 25518754 | 10280 | 25: 25644509 | 10280 | 25: 25729230 |
| 10280 | 25: 25764911 | 10281 | 25: 26048984 | 10283 | 25: 26491613 | 10283 | 25: 26491635 | 10283 | 25: 26491678 |
| 10283 | 25: 26491720 | 10283 | 25: 26491762 | 10284 | 25: 26492004 | 10284 | 25: 26492080 | 10284 | 25: 26492086 |
| 10284 | 25: 26492143 | 10284 | 25: 26493056 | 10284 | 25: 26493215 | 10284 | 25: 26493378 | 10285 | 25: 26873278 |
| 10285 | 25: 26879815 | 10286 | 25: 26957845 | 10286 | 25: 27074581 | 10286 | 25: 27078879 | 10286 | 25: 27079915 |
| 10286 | 25: 27081813 | 10288 | 25: 27471780 | 10288 | 25: 27505633 | 10288 | 25: 27529561 | 10288 | 25: 27546363 |
| 10288 | 25: 27577974 | 10288 | 25: 27610631 | 10289 | 25: 27614146 | 10289 | 25: 27621694 | 10289 | 25: 27623762 |
| 10289 | 25: 27634139 | 10289 | 25: 27647855 | 10289 | 25: 27651029 | 10289 | 25: 27654242 | 10289 | 25: 27671337 |
| 10289 | 25: 27672683 | 10289 | 25: 27677306 | 10290 | 25: 27724958 | 10290 | 25: 27728373 | 10290 | 25: 27731091 |
| 10290 | 25: 27734591 | 10290 | 25: 27737084 | 10290 | 25: 27743420 | 10290 | 25: 27747175 | 10291 | 25: 27747494 |
| 10291 | 25: 27747510 | 10291 | 25: 27748521 | 10291 | 25: 27749865 | 10291 | 25: 27749919 | 10291 | 25: 27749952 |
| 10291 | 25: 27749994 | 10291 | 25: 27750315 | 10291 | 25: 27750769 | 10291 | 25: 27751432 | 10292 | 25: 27790340 |
| 10293 | 25: 28024158 | 10293 | 25: 28057161 | 10293 | 25: 28188815 | 10294 | 25: 28274850 | 10294 | 25: 28376458 |
| 10295 | 25: 28539043 | 10295 | 25: 28760002 | 10297 | 25: 29158123 | 10298 | 25: 29422226 | 10298 | 25: 29507318 |
| 10299 | 25: 29574598 | 10299 | 25: 29653196 | 10300 | 25: 29711516 | 10301 | 25: 29924846 | 10302 | 25: 29960827 |
| 10302 | 25: 29983402 | 10302 | 25: 30108430 | 10303 | 25: 30188316 | 10306 | 25: 31059775 | 10308 | 25: 31397966 |
| 10308 | 25: 31397972 | 10312 | 25: 32101198 | 10312 | 25: 32115103 | 10312 | 25: 32122180 | 10313 | 25: 32225777 |
| 10314 | 25: 32620702 | 10314 | 25: 32636956 | 10315 | 25: 32651385 | 10315 | 25: 32784599 | 10315 | 25: 32785820 |
| 10315 | 25: 32838124 | 10316 | 25: 33064837 | 10316 | 25: 33068854 | 10317 | 25: 33278840 | 10317 | 25: 33325832 |
| 10318 | 25: 33472178 | 10319 | 25: 33614940 | 10319 | 25: 33626966 | 10320 | 25: 33862553 | 10320 | 25: 33918451 |
| 10320 | 25: 33918614 | 10321 | 25: 33933997 | 10321 | 25: 33986857 | 10322 | 25: 34165756 | 10322 | 25: 34324511 |
| 10323 | 25: 34373185 | 10323 | 25: 34380738 | 10323 | 25: 34390163 | 10323 | 25: 34415206 | 10323 | 25: 34416256 |
| 10323 | 25: 34423242 | 10323 | 25: 34430800 | 10324 | 25: 34512804 | 10324 | 25: 34523864 | 10324 | 25: 34541030 |
| 10324 | 25: 34607593 | 10324 | 25: 34616589 | 10325 | 25: 34627570 | 10325 | 25: 34729070 | 10326 | 25: 34979341 |
| 10327 | 25: 34987197 | 10327 | 25: 35035356 | 10327 | 25: 35047480 | 10327 | 25: 35162155 | 10328 | 25: 35220858 |
| 10328 | 25: 35223524 | 10328 | 25: 35224634 | 10329 | 25: 35416991 | 10329 | 25: 35510566 | 10329 | 25: 35538895 |
| 10329 | 25: 35539311 | 10330 | 25: 35574079 | 10330 | 25: 35596429 | 10330 | 25: 35632501 | 10331 | 25: 35695366 |
| 10331 | 25: 35695667 | 10331 | 25: 35695717 | 10331 | 25: 35696225 | 10331 | 25: 35696282 | 10331 | 25: 35697157 |
| 10331 | 25: 35723853 | 10331 | 25: 35726457 | 10331 | 25: 35737565 | 10332 | 25: 35767708 | 10332 | 25: 35774507 |
| 10332 | 25: 35775008 | 10332 | 25: 35784841 | 10332 | 25: 35868596 | 10333 | 25: 35874518 | 10333 | 25: 35930268 |
| 10333 | 25: 35960525 | 10334 | 25: 36192757 | 10334 | 25: 36214845 | 10334 | 25: 36214858 | 10334 | 25: 36215138 |
| 10335 | 25: 36215175 | 10335 | 25: 36215182 | 10335 | 25: 36215303 | 10335 | 25: 36215758 | 10335 | 25: 36215759 |
| 10335 | 25: 36216611 | 10335 | 25: 36216901 | 10335 | 25: 36216911 | 10335 | 25: 36217079 | 10335 | 25: 36217150 |
| 10336 | 25: 36217198 | 10336 | 25: 36217712 | 10336 | 25: 36217751 | 10336 | 25: 36225793 | 10336 | 25: 36225817 |
| 10336 | 25: 36246266 | 10336 | 25: 36249624 | 10337 | 25: 36344903 | 10337 | 25: 36350043 | 10337 | 25: 36360014 |
| 10337 | 25: 36450152 | 10338 | 25: 36450202 | 10338 | 25: 36512847 | 10338 | 25: 36531061 | 10338 | 25: 36535022 |
| 10338 | 25: 36538745 | 10338 | 25: 36584800 | 10339 | 25: 36811728 | 10339 | 25: 36971262 | 10339 | 25: 36978649 |
| 10340 | 25: 36985203 | 10340 | 25: 37031840 | 10340 | 25: 37034930 | 10340 | 25: 37043595 | 10340 | 25: 37046117 |
| 10340 | 25: 37060161 | 10340 | 25: 37061145 | 10341 | 25: 37136607 | 10341 | 25: 37136612 | 10341 | 25: 37138640 |
| 10341 | 25: 37138651 | 10341 | 25: 37138668 | 10341 | 25: 37149974 | 10342 | 25: 37184274 | 10342 | 25: 37205234 |
| 10342 | 25: 37227790 | 10342 | 25: 37241594 | 10342 | 25: 37242352 | 10342 | 25: 37242939 | 10343 | 25: 37242955 |
| 10343 | 25: 37242995 | 10343 | 25: 37243078 | 10343 | 25: 37252529 | 10343 | 25: 37356346 | 10344 | 25: 37389979 |
| 10346 | 25: 37729407 | 10346 | 25: 37784956 | 10347 | 25: 37964578 | 10348 | 25: 38114076 | 10348 | 25: 38114479 |
| 10348 | 25: 38117206 | 10348 | 25: 38118273 | 10348 | 25: 38126701 | 10348 | 25: 38166852 | 10348 | 25: 38190732 |
| 10349 | 25: 38227705 | 10349 | 25: 38233097 | 10349 | 25: 38300677 | 10349 | 25: 38300772 | 10349 | 25: 38300781 |
| 10350 | 25: 38363440 | 10350 | 25: 38394640 | 10350 | 25: 38394741 | 10350 | 25: 38489542 | 10352 | 25: 38856435 |
| 10352 | 25: 38930854 | 10353 | 25: 39028696 | 10353 | 25: 39045580 | 10354 | 25: 39300696 | 10354 | 25: 39317059 |
| 10354 | 25: 39342146 | 10354 | 25: 39345282 | 10354 | 25: 39449897 | 10355 | 25: 39552784 | 10357 | 25: 39918879 |
| 10359 | 25: 40240409 | 10359 | 25: 40240500 | 10360 | 25: 40328748 | 10360 | 25: 40396102 | 10360 | 25: 40553090 |
| 10361 | 25: 40614314 | 10362 | 25: 40720968 | 10362 | 25: 40758343 | 10363 | 25: 40876573 | 10363 | 25: 40901328 |
| 10363 | 25: 40903884 | 10363 | 25: 40948048 | 10364 | 25: 40954468 | 10364 | 25: 40956329 | 10364 | 25: 40960034 |
| 10364 | 25: 40960925 | 10364 | 25: 40979131 | 10364 | 25: 40982964 | 10364 | 25: 40991964 | 10364 | 25: 40998163 |
| 10364 | 25: 41007519 | 10365 | 25: 41009480 | 10365 | 25: 41012986 | 10365 | 25: 41014477 | 10365 | 25: 41015047 |
| 10365 | 25: 41022922 | 10365 | 25: 41028182 | 10365 | 25: 41031444 | 10365 | 25: 41033389 | 10365 | 25: 41034653 |
| 10365 | 25: 41039109 | 10366 | 25: 41046191 | 10366 | 25: 41066639 | 10366 | 25: 41070067 | 10366 | 25: 41074859 |
| 10367 | 25: 41373314 | 10367 | 25: 41416055 | 10367 | 25: 41479611 | 10367 | 25: 41496081 | 10368 | 25: 41719279 |
| 10368 | 25: 41758231 | 10369 | 25: 42027128 | 10369 | 25: 42031008 | 10370 | 25: 42287133 | 10370 | 25: 42290491 |
| 10370 | 25: 42292572 | 10371 | 26: 840412 | 10372 | 26: 1046523 | 10373 | 26: 1202224 | 10374 | 26: 1527005 |
| 10375 | 26: 1774152 | 10375 | 26: 1837613 | 10375 | 26: 1837679 | 10375 | 26: 1837715 | 10378 | 26: 2789785 |
| 10379 | 26: 2795689 | 10379 | 26: 2807861 | 10379 | 26: 2809033 | 10379 | 26: 2810402 | 10379 | 26: 2810457 |
| 10379 | 26: 2810725 | 10379 | 26: 2811451 | 10379 | 26: 2811491 | 10379 | 26: 2811596 | 10379 | 26: 2811603 |
| 10380 | 26: 2811953 | 10380 | 26: 2812213 | 10380 | 26: 2812496 | 10380 | 26: 2834801 | 10380 | 26: 2836372 |
| 10380 | 26: 2838470 | 10380 | 26: 2839812 | 10380 | 26: 2843878 | 10380 | 26: 2847631 | 10380 | 26: 2853005 |
| 10381 | 26: 2869288 | 10381 | 26: 2912970 | 10382 | 26: 3233006 | 10382 | 26: 3276739 | 10382 | 26: 3276794 |
| 10382 | 26: 3347635 | 10382 | 26: 3347724 | 10385 | 26: 3975478 | 10387 | 26: 4592577 | 10389 | 26: 4967710 |
| 10389 | 26: 5039459 | 10390 | 26: 5408963 | 10395 | 26: 6941203 | 10395 | 26: 6944017 | 10396 | 26: 7431785 |
| 10396 | 26: 7456216 | 10396 | 26: 7484390 | 10397 | 26: 7488314 | 10397 | 26: 7495226 | 10397 | 26: 7510638 |
| 10397 | 26: 7591687 | 10397 | 26: 7696472 | 10398 | 26: 7696485 | 10398 | 26: 7727559 | 10399 | 26: 8027593 |
| 10399 | 26: 8048518 | 10400 | 26: 8204339 | 10401 | 26: 8516699 | 10401 | 26: 8538464 | 10402 | 26: 8757164 |
| 10403 | 26: 8943593 | 10403 | 26: 8960699 | 10404 | 26: 9137009 | 10405 | 26: 9666870 | 10406 | 26: 9801561 |
| 10407 | 26: 10180467 | 10407 | 26: 10180516 | 10408 | 26: 10195205 | 10408 | 26: 10195875 | 10408 | 26: 10196016 |
| 10408 | 26: 10197224 | 10408 | 26: 10201006 | 10408 | 26: 10203129 | 10408 | 26: 10208363 | 10408 | 26: 10212949 |
| 10408 | 26: 10214107 | 10408 | 26: 10221720 | 10409 | 26: 10302490 | 10409 | 26: 10305675 | 10410 | 26: 10616738 |
| 10411 | 26: 10731742 | 10412 | 26: 11167367 | 10413 | 26: 11511468 | 10414 | 26: 11702998 | 10414 | 26: 11703000 |
| 10414 | 26: 11703003 | 10414 | 26: 11703009 | 10415 | 26: 11703013 | 10415 | 26: 11703046 | 10415 | 26: 11703059 |
| 10415 | 26: 11703143 | 10417 | 26: 12409311 | 10417 | 26: 12493226 | 10417 | 26: 12493314 | 10417 | 26: 12576243 |
| 10417 | 26: 12663124 | 10418 | 26: 12857727 | 10419 | 26: 12905617 | 10419 | 26: 12936955 | 10419 | 26: 13005236 |
| 10420 | 26: 13185986 | 10420 | 26: 13213326 | 10421 | 26: 13237530 | 10421 | 26: 13242646 | 10421 | 26: 13253767 |
| 10422 | 26: 13348439 | 10422 | 26: 13439428 | 10422 | 26: 13487765 | 10423 | 26: 13487832 | 10423 | 26: 13628978 |
| 10423 | 26: 13632894 | 10423 | 26: 13666033 | 10424 | 26: 13818460 | 10424 | 26: 13935413 | 10425 | 26: 14204588 |
| 10425 | 26: 14204609 | 10427 | 26: 14670368 | 10427 | 26: 14688221 | 10427 | 26: 14729665 | 10427 | 26: 14889689 |
| 10427 | 26: 14890394 | 10428 | 26: 14896553 | 10428 | 26: 15033159 | 10429 | 26: 15470094 | 10431 | 26: 15856645 |
| 10431 | 26: 15856778 | 10431 | 26: 15865811 | 10431 | 26: 15866192 | 10431 | 26: 15867927 | 10431 | 26: 15867943 |
| 10431 | 26: 15876906 | 10434 | 26: 16752906 | 10434 | 26: 16926415 | 10434 | 26: 16926501 | 10435 | 26: 16951340 |
| 10435 | 26: 17033041 | 10435 | 26: 17041251 | 10435 | 26: 17100094 | 10436 | 26: 17127186 | 10436 | 26: 17156417 |
| 10436 | 26: 17197835 | 10436 | 26: 17325459 | 10437 | 26: 17482702 | 10437 | 26: 17539828 | 10438 | 26: 17628730 |
| 10438 | 26: 17628780 | 10438 | 26: 17685991 | 10438 | 26: 17691480 | 10438 | 26: 17691492 | 10439 | 26: 17746468 |
| 10439 | 26: 17781937 | 10439 | 26: 17879952 | 10440 | 26: 17928505 | 10440 | 26: 17965085 | 10441 | 26: 18209480 |
| 10441 | 26: 18211289 | 10441 | 26: 18355673 | 10442 | 26: 18490628 | 10442 | 26: 18494442 | 10442 | 26: 18521176 |
| 10442 | 26: 18528824 | 10443 | 26: 18651237 | 10443 | 26: 18695230 | 10443 | 26: 18702491 | 10443 | 26: 18728512 |
| 10443 | 26: 18740541 | 10444 | 26: 18782784 | 10444 | 26: 18869630 | 10444 | 26: 18885108 | 10444 | 26: 18907964 |
| 10444 | 26: 18929393 | 10445 | 26: 18970293 | 10445 | 26: 19009189 | 10446 | 26: 19238259 | 10447 | 26: 19438464 |
| 10447 | 26: 19452547 | 10447 | 26: 19494159 | 10448 | 26: 19510054 | 10448 | 26: 19547291 | 10448 | 26: 19597530 |
| 10449 | 26: 20104927 | 10449 | 26: 20143627 | 10451 | 26: 20594269 | 10452 | 26: 21094234 | 10453 | 26: 21239671 |
| 10453 | 26: 21254361 | 10456 | 26: 21784164 | 10456 | 26: 21862417 | 10456 | 26: 21918010 | 10456 | 26: 21964449 |
| 10456 | 26: 22002686 | 10457 | 26: 22058857 | 10457 | 26: 22063661 | 10457 | 26: 22251251 | 10458 | 26: 22554517 |
| 10458 | 26: 22581259 | 10458 | 26: 22606617 | 10458 | 26: 22658743 | 10459 | 26: 22659438 | 10461 | 26: 23328557 |
| 10461 | 26: 23398930 | 10461 | 26: 23503177 | 10462 | 26: 23741250 | 10463 | 26: 24028517 | 10463 | 26: 24127485 |
| 10463 | 26: 24135884 | 10464 | 26: 24188588 | 10464 | 26: 24239524 | 10464 | 26: 24286733 | 10464 | 26: 24287516 |
| 10464 | 26: 24310631 | 10465 | 26: 24590237 | 10466 | 26: 24630200 | 10466 | 26: 24764833 | 10466 | 26: 24781597 |
| 10466 | 26: 24818702 | 10467 | 26: 24819776 | 10467 | 26: 24820679 | 10467 | 26: 24823044 | 10467 | 26: 24826317 |
| 10467 | 26: 24829772 | 10467 | 26: 24831082 | 10467 | 26: 24831934 | 10467 | 26: 24850967 | 10468 | 26: 24867806 |
| 10468 | 26: 24877750 | 10468 | 26: 24920605 | 10470 | 26: 25371117 | 10470 | 26: 25377110 | 10471 | 26: 25408794 |
| 10471 | 26: 25438245 | 10472 | 26: 25560669 | 10472 | 26: 25586394 | 10472 | 26: 25601218 | 10472 | 26: 25624158 |
| 10472 | 26: 25628586 | 10472 | 26: 25632049 | 10472 | 26: 25636772 | 10472 | 26: 25637820 | 10472 | 26: 25640830 |
| 10473 | 26: 25643571 | 10473 | 26: 25645847 | 10473 | 26: 25646718 | 10473 | 26: 25649902 | 10473 | 26: 25653351 |
| 10473 | 26: 25664984 | 10473 | 26: 25690676 | 10474 | 26: 25738398 | 10474 | 26: 25884432 | 10474 | 26: 25905471 |
| 10474 | 26: 25923067 | 10474 | 26: 25924666 | 10475 | 26: 26022373 | 10475 | 26: 26108845 | 10476 | 26: 26209053 |
| 10476 | 26: 26211205 | 10477 | 26: 26461336 | 10479 | 26: 26855141 | 10480 | 26: 27069339 | 10482 | 26: 27558387 |
| 10482 | 26: 27572772 | 10483 | 26: 27736462 | 10483 | 26: 27736471 | 10483 | 26: 27736612 | 10485 | 26: 28197657 |
| 10487 | 26: 28950952 | 10487 | 26: 29088929 | 10488 | 26: 29186399 | 10489 | 26: 29513286 | 10491 | 26: 29986030 |
| 10491 | 26: 30197525 | 10491 | 26: 30197564 | 10491 | 26: 30197578 | 10492 | 26: 30197681 | 10493 | 26: 30889276 |
| 10493 | 26: 30892846 | 10494 | 26: 30950051 | 10495 | 26: 31271680 | 10495 | 26: 31311514 | 10495 | 26: 31323087 |
| 10496 | 26: 31350194 | 10496 | 26: 31368850 | 10496 | 26: 31379154 | 10496 | 26: 31411730 | 10496 | 26: 31483017 |
| 10497 | 26: 31801170 | 10499 | 26: 32178829 | 10499 | 26: 32210509 | 10500 | 26: 32484431 | 10500 | 26: 32527484 |
| 10501 | 26: 32691372 | 10501 | 26: 32756815 | 10502 | 26: 32879086 | 10503 | 26: 33122620 | 10504 | 26: 33282896 |
| 10504 | 26: 33328268 | 10504 | 26: 33359761 | 10504 | 26: 33380765 | 10504 | 26: 33400303 | 10505 | 26: 33526762 |
| 10505 | 26: 33530073 | 10505 | 26: 33560198 | 10506 | 26: 33837346 | 10506 | 26: 33901952 | 10507 | 26: 33914479 |
| 10507 | 26: 33922739 | 10508 | 26: 34087101 | 10508 | 26: 34109859 | 10508 | 26: 34125393 | 10508 | 26: 34140428 |
| 10508 | 26: 34145240 | 10508 | 26: 34148000 | 10509 | 26: 34318487 | 10509 | 26: 34354045 | 10510 | 26: 34633936 |
| 10511 | 26: 34718279 | 10511 | 26: 34746698 | 10511 | 26: 34746782 | 10511 | 26: 34746817 | 10511 | 26: 34746843 |
| 10511 | 26: 34746854 | 10511 | 26: 34746898 | 10511 | 26: 34755555 | 10512 | 26: 34829732 | 10512 | 26: 34832045 |
| 10512 | 26: 35009461 | 10513 | 26: 35045257 | 10513 | 26: 35119651 | 10513 | 26: 35152534 | 10514 | 26: 35387482 |
| 10514 | 26: 35446688 | 10515 | 26: 35473524 | 10515 | 26: 35703319 | 10515 | 26: 35730641 | 10515 | 26: 35730653 |
| 10515 | 26: 35730692 | 10515 | 26: 35730695 | 10516 | 26: 35894255 | 10518 | 26: 36442085 | 10518 | 26: 36465890 |
| 10518 | 26: 36466008 | 10519 | 26: 36782486 | 10520 | 26: 36868486 | 10520 | 26: 36875653 | 10520 | 26: 36881092 |
| 10520 | 26: 36895816 | 10520 | 26: 36899560 | 10520 | 26: 36899693 | 10520 | 26: 36955993 | 10520 | 26: 36966986 |
| 10521 | 26: 36968918 | 10521 | 26: 36968980 | 10521 | 26: 36969055 | 10522 | 26: 37095749 | 10523 | 26: 37812473 |
| 10524 | 26: 37879370 | 10524 | 26: 37908929 | 10524 | 26: 38110713 | 10526 | 26: 38674064 | 10527 | 26: 38851055 |
| 10527 | 26: 38854747 | 10527 | 26: 38875810 | 10527 | 26: 38877239 | 10527 | 26: 38881457 | 10528 | 26: 38992651 |
| 10528 | 26: 39006604 | 10529 | 26: 39098131 | 10529 | 26: 39189961 | 10529 | 26: 39196795 | 10529 | 26: 39202461 |
| 10530 | 26: 39305962 | 10530 | 26: 39426186 | 10530 | 26: 39426206 | 10530 | 26: 39426264 | 10530 | 26: 39485796 |
| 10531 | 26: 39494577 | 10531 | 26: 39507180 | 10531 | 26: 39538069 | 10531 | 26: 39573736 | 10532 | 26: 39783330 |
| 10532 | 26: 40032776 | 10533 | 26: 40040946 | 10533 | 26: 40070046 | 10534 | 26: 40441942 | 10535 | 26: 40786447 |
| 10536 | 26: 41019385 | 10536 | 26: 41076854 | 10536 | 26: 41096640 | 10537 | 26: 41390989 | 10539 | 26: 41918819 |
| 10539 | 26: 41926321 | 10540 | 26: 42008210 | 10540 | 26: 42024771 | 10540 | 26: 42049951 | 10540 | 26: 42090283 |
| 10540 | 26: 42092905 | 10541 | 26: 42109007 | 10541 | 26: 42309356 | 10541 | 26: 42330778 | 10542 | 26: 42350916 |
| 10543 | 26: 42642591 | 10543 | 26: 42660447 | 10543 | 26: 42757714 | 10544 | 26: 43041112 | 10545 | 26: 43062676 |
| 10546 | 26: 43622892 | 10547 | 26: 43740696 | 10547 | 26: 43816442 | 10548 | 26: 44064506 | 10548 | 26: 44083273 |
| 10548 | 26: 44083285 | 10549 | 26: 44083315 | 10549 | 26: 44083393 | 10549 | 26: 44083680 | 10550 | 26: 44255933 |
| 10550 | 26: 44404294 | 10550 | 26: 44481659 | 10551 | 26: 44674003 | 10551 | 26: 44723893 | 10552 | 26: 45008042 |
| 10553 | 26: 45209100 | 10553 | 26: 45264287 | 10554 | 26: 45324531 | 10555 | 26: 45412608 | 10556 | 26: 45540372 |
| 10556 | 26: 45540404 | 10556 | 26: 45540418 | 10556 | 26: 45540427 | 10556 | 26: 45542714 | 10556 | 26: 45565711 |
| 10557 | 26: 45729275 | 10558 | 26: 46112041 | 10559 | 26: 46315775 | 10559 | 26: 46317103 | 10559 | 26: 46317812 |
| 10559 | 26: 46318923 | 10560 | 26: 46353080 | 10560 | 26: 46421801 | 10560 | 26: 46452461 | 10560 | 26: 46466580 |
| 10560 | 26: 46471616 | 10560 | 26: 46479016 | 10561 | 26: 46559579 | 10563 | 26: 47028707 | 10564 | 26: 47333154 |
| 10564 | 26: 47361635 | 10564 | 26: 47427206 | 10565 | 26: 47512133 | 10565 | 26: 47512681 | 10565 | 26: 47615883 |
| 10566 | 26: 47650142 | 10566 | 26: 47772970 | 10569 | 26: 48408440 | 10569 | 26: 48479152 | 10571 | 26: 48865592 |
| 10571 | 26: 48866736 | 10571 | 26: 48968839 | 10571 | 26: 49004191 | 10574 | 26: 49515195 | 10574 | 26: 49563646 |
| 10575 | 26: 49563747 | 10579 | 26: 50590249 | 10579 | 26: 50709285 | 10579 | 26: 50733543 | 10580 | 26: 50733621 |
| 10580 | 26: 50823585 | 10580 | 26: 50825362 | 10580 | 26: 50830075 | 10581 | 26: 51009547 | 10581 | 26: 51009628 |
| 10581 | 26: 51127593 | 10582 | 26: 51175800 | 10583 | 26: 51422136 | 10584 | 26: 51748308 | 10584 | 26: 51784240 |
| 10584 | 26: 51824417 | 10584 | 26: 51837838 | 10584 | 26: 51856701 | 10585 | 26: 51990316 | 10586 | 27: 504917 |
| 10586 | 27: 504918 | 10586 | 27: 504943 | 10586 | 27: 622508 | 10587 | 27: 1220407 | 10588 | 27: 1285546 |
| 10588 | 27: 1330181 | 10588 | 27: 1355435 | 10589 | 27: 1355462 | 10589 | 27: 1362304 | 10589 | 27: 1362959 |
| 10589 | 27: 1375496 | 10589 | 27: 1376218 | 10589 | 27: 1382415 | 10589 | 27: 1384981 | 10589 | 27: 1385318 |
| 10589 | 27: 1385323 | 10589 | 27: 1385392 | 10590 | 27: 1385425 | 10590 | 27: 1385455 | 10590 | 27: 1395995 |
| 10590 | 27: 1401856 | 10590 | 27: 1401865 | 10590 | 27: 1405119 | 10590 | 27: 1407822 | 10590 | 27: 1453147 |
| 10591 | 27: 1475170 | 10593 | 27: 2200746 | 10593 | 27: 2232971 | 10594 | 27: 2375493 | 10595 | 27: 2508576 |
| 10597 | 27: 2988336 | 10600 | 27: 3776760 | 10600 | 27: 3787074 | 10600 | 27: 3788517 | 10600 | 27: 3790934 |
| 10600 | 27: 3805789 | 10602 | 27: 4234197 | 10604 | 27: 4820720 | 10604 | 27: 4843559 | 10605 | 27: 4942987 |
| 10605 | 27: 4946073 | 10605 | 27: 4962502 | 10605 | 27: 4964043 | 10605 | 27: 5085626 | 10606 | 27: 5098545 |
| 10606 | 27: 5158253 | 10606 | 27: 5391087 | 10607 | 27: 5392011 | 10607 | 27: 5395069 | 10607 | 27: 5396567 |
| 10607 | 27: 5399954 | 10607 | 27: 5401556 | 10607 | 27: 5402389 | 10607 | 27: 5403342 | 10607 | 27: 5404401 |
| 10607 | 27: 5418944 | 10608 | 27: 5421637 | 10608 | 27: 5428881 | 10608 | 27: 5430538 | 10608 | 27: 5470087 |
| 10608 | 27: 5478901 | 10608 | 27: 5582204 | 10609 | 27: 5587883 | 10609 | 27: 5611632 | 10609 | 27: 5792112 |
| 10610 | 27: 5827416 | 10610 | 27: 5974973 | 10610 | 27: 5988748 | 10610 | 27: 6025859 | 10610 | 27: 6032871 |
| 10611 | 27: 6060484 | 10611 | 27: 6087111 | 10614 | 27: 7631148 | 10614 | 27: 7653356 | 10614 | 27: 7733608 |
| 10615 | 27: 7750098 | 10615 | 27: 7770345 | 10615 | 27: 7791683 | 10615 | 27: 7807058 | 10615 | 27: 7813201 |
| 10616 | 27: 7920370 | 10616 | 27: 7920432 | 10616 | 27: 7920451 | 10616 | 27: 7958336 | 10617 | 27: 7975782 |
| 10617 | 27: 7980889 | 10617 | 27: 8132488 | 10617 | 27: 8191553 | 10618 | 27: 8289670 | 10618 | 27: 8359908 |
| 10618 | 27: 8470036 | 10618 | 27: 8476834 | 10619 | 27: 8575128 | 10619 | 27: 8612967 | 10623 | 27: 9145723 |
| 10624 | 27: 9202230 | 10624 | 27: 9202257 | 10624 | 27: 9202263 | 10624 | 27: 9202295 | 10625 | 27: 9488574 |
| 10630 | 27: 11255396 | 10630 | 27: 11262348 | 10632 | 27: 11669357 | 10632 | 27: 11736207 | 10634 | 27: 12235101 |
| 10634 | 27: 12270834 | 10637 | 27: 12976567 | 10637 | 27: 13162684 | 10638 | 27: 13319931 | 10639 | 27: 13521583 |
| 10640 | 27: 13703268 | 10640 | 27: 13763652 | 10641 | 27: 13769140 | 10641 | 27: 13864569 | 10645 | 27: 14058907 |
| 10645 | 27: 14089539 | 10646 | 27: 14337753 | 10647 | 27: 14505664 | 10647 | 27: 14619598 | 10650 | 27: 15114953 |
| 10650 | 27: 15209444 | 10651 | 27: 15342336 | 10651 | 27: 15374174 | 10652 | 27: 15616842 | 10653 | 27: 15679545 |
| 10653 | 27: 15679907 | 10653 | 27: 15679986 | 10653 | 27: 15679987 | 10653 | 27: 15724630 | 10655 | 27: 16091144 |
| 10656 | 27: 16231107 | 10656 | 27: 16231139 | 10656 | 27: 16250819 | 10656 | 27: 16262844 | 10657 | 27: 16289197 |
| 10657 | 27: 16289212 | 10657 | 27: 16310423 | 10657 | 27: 16311908 | 10657 | 27: 16311992 | 10658 | 27: 16448657 |
| 10658 | 27: 16475268 | 10658 | 27: 16527145 | 10658 | 27: 16527632 | 10659 | 27: 16545436 | 10659 | 27: 16626449 |
| 10660 | 27: 16876243 | 10661 | 27: 17114420 | 10663 | 27: 17756331 | 10663 | 27: 17825429 | 10664 | 27: 17900781 |
| 10668 | 27: 18743674 | 10670 | 27: 19184326 | 10670 | 27: 19226515 | 10671 | 27: 19413103 | 10672 | 27: 19615386 |
| 10672 | 27: 19628648 | 10672 | 27: 19638432 | 10672 | 27: 19643687 | 10672 | 27: 19643703 | 10672 | 27: 19656115 |
| 10673 | 27: 19709555 | 10673 | 27: 19715294 | 10673 | 27: 19715322 | 10673 | 27: 19743997 | 10673 | 27: 19759721 |
| 10674 | 27: 19916100 | 10675 | 27: 20275887 | 10676 | 27: 20403248 | 10677 | 27: 21028770 | 10679 | 27: 21603965 |
| 10680 | 27: 21736051 | 10681 | 27: 21968621 | 10682 | 27: 21988370 | 10682 | 27: 21988471 | 10682 | 27: 22006706 |
| 10683 | 27: 22555224 | 10686 | 27: 23217717 | 10686 | 27: 23219768 | 10686 | 27: 23235008 | 10686 | 27: 23291834 |
| 10688 | 27: 23591760 | 10688 | 27: 23763903 | 10689 | 27: 23820540 | 10689 | 27: 23900777 | 10690 | 27: 23960140 |
| 10690 | 27: 23969325 | 10690 | 27: 23997295 | 10690 | 27: 24130313 | 10691 | 27: 24151993 | 10691 | 27: 24152004 |
| 10691 | 27: 24203304 | 10691 | 27: 24203421 | 10691 | 27: 24222816 | 10692 | 27: 24521008 | 10693 | 27: 24725230 |
| 10694 | 27: 24839161 | 10694 | 27: 24839261 | 10696 | 27: 25252926 | 10696 | 27: 25328242 | 10697 | 27: 25416389 |
| 10698 | 27: 25562534 | 10698 | 27: 25566427 | 10698 | 27: 25728403 | 10699 | 27: 26089588 | 10700 | 27: 26265901 |
| 10700 | 27: 26306516 | 10700 | 27: 26385003 | 10701 | 27: 26490500 | 10702 | 27: 26581172 | 10702 | 27: 26586255 |
| 10702 | 27: 26593729 | 10703 | 27: 26832000 | 10704 | 27: 26846204 | 10704 | 27: 26878354 | 10704 | 27: 26886076 |
| 10704 | 27: 26894469 | 10704 | 27: 26904559 | 10704 | 27: 26906127 | 10704 | 27: 26924212 | 10706 | 27: 27222640 |
| 10706 | 27: 27223927 | 10706 | 27: 27223929 | 10706 | 27: 27224038 | 10706 | 27: 27224048 | 10706 | 27: 27224849 |
| 10706 | 27: 27224850 | 10708 | 27: 27637602 | 10709 | 27: 27970577 | 10710 | 27: 28171888 | 10713 | 27: 28946541 |
| 10714 | 27: 29252315 | 10715 | 27: 29278521 | 10715 | 27: 29296483 | 10715 | 27: 29312772 | 10715 | 27: 29327883 |
| 10715 | 27: 29327914 | 10716 | 27: 29327998 | 10718 | 27: 30202829 | 10719 | 27: 30202912 | 10719 | 27: 30226393 |
| 10719 | 27: 30244634 | 10722 | 27: 31002073 | 10722 | 27: 31002095 | 10722 | 27: 31002129 | 10723 | 27: 31078802 |
| 10724 | 27: 31274065 | 10726 | 27: 31729965 | 10726 | 27: 31841660 | 10726 | 27: 31841704 | 10727 | 27: 31897740 |
| 10729 | 27: 32226765 | 10729 | 27: 32254312 | 10729 | 27: 32265770 | 10730 | 27: 32268130 | 10730 | 27: 32276493 |
| 10731 | 27: 32518470 | 10731 | 27: 32518472 | 10731 | 27: 32518552 | 10731 | 27: 32583194 | 10732 | 27: 32727516 |
| 10732 | 27: 32727582 | 10732 | 27: 32727603 | 10732 | 27: 32727620 | 10732 | 27: 32778474 | 10732 | 27: 32793041 |
| 10733 | 27: 32802126 | 10734 | 27: 33001000 | 10734 | 27: 33009928 | 10734 | 27: 33086268 | 10735 | 27: 33233127 |
| 10735 | 27: 33265366 | 10735 | 27: 33272590 | 10736 | 27: 33421612 | 10736 | 27: 33629831 | 10736 | 27: 33633260 |
| 10737 | 27: 33872186 | 10737 | 27: 33879901 | 10737 | 27: 33913097 | 10738 | 27: 34127235 | 10739 | 27: 34635911 |
| 10740 | 27: 34637563 | 10740 | 27: 34752720 | 10741 | 27: 34981815 | 10741 | 27: 35014810 | 10741 | 27: 35015975 |
| 10741 | 27: 35016903 | 10741 | 27: 35018328 | 10741 | 27: 35019984 | 10741 | 27: 35021367 | 10742 | 27: 35026180 |
| 10742 | 27: 35032183 | 10742 | 27: 35042729 | 10742 | 27: 35045715 | 10742 | 27: 35047820 | 10742 | 27: 35052712 |
| 10742 | 27: 35053785 | 10742 | 27: 35054739 | 10742 | 27: 35058765 | 10743 | 27: 35060134 | 10743 | 27: 35064362 |
| 10743 | 27: 35065072 | 10743 | 27: 35072059 | 10743 | 27: 35075103 | 10743 | 27: 35079318 | 10743 | 27: 35080042 |
| 10743 | 27: 35098005 | 10744 | 27: 35103784 | 10744 | 27: 35121417 | 10744 | 27: 35122313 | 10744 | 27: 35128376 |
| 10744 | 27: 35138175 | 10744 | 27: 35184790 | 10747 | 27: 35555630 | 10747 | 27: 35599360 | 10748 | 27: 35782254 |
| 10748 | 27: 35824524 | 10748 | 27: 35824606 | 10749 | 27: 35975385 | 10749 | 27: 35978336 | 10750 | 27: 36138112 |
| 10750 | 27: 36216370 | 10752 | 27: 36576641 | 10753 | 27: 36751396 | 10753 | 27: 36906500 | 10753 | 27: 37106071 |
| 10754 | 27: 37127877 | 10754 | 27: 37172084 | 10754 | 27: 37192870 | 10754 | 27: 37195004 | 10754 | 27: 37274132 |
| 10755 | 27: 37275861 | 10755 | 27: 37293040 | 10755 | 27: 37297500 | 10756 | 27: 37374389 | 10756 | 27: 37486971 |
| 10756 | 27: 37493520 | 10756 | 27: 37521766 | 10757 | 27: 37573787 | 10757 | 27: 37620840 | 10757 | 27: 37630299 |
| 10757 | 27: 37639061 | 10758 | 27: 37746356 | 10759 | 27: 37827637 | 10759 | 27: 37864645 | 10760 | 27: 38151557 |
| 10760 | 27: 38168061 | 10761 | 27: 38327204 | 10761 | 27: 38432520 | 10762 | 27: 38491615 | 10763 | 27: 38861489 |
| 10764 | 27: 39015604 | 10764 | 27: 39015763 | 10764 | 27: 39015976 | 10766 | 27: 39667527 | 10766 | 27: 39668431 |
| 10767 | 27: 39780803 | 10768 | 27: 40004933 | 10769 | 27: 40356156 | 10769 | 27: 40357028 | 10769 | 27: 40414746 |
| 10770 | 27: 40674026 | 10770 | 27: 40868181 | 10771 | 27: 41044067 | 10772 | 27: 41178088 | 10773 | 27: 41583577 |
| 10774 | 27: 41702146 | 10775 | 27: 41886684 | 10776 | 27: 42166403 | 10776 | 27: 42228982 | 10776 | 27: 42242635 |
| 10777 | 27: 42427599 | 10777 | 27: 42462522 | 10777 | 27: 42487443 | 10778 | 27: 42794556 | 10781 | 27: 43428164 |
| 10783 | 27: 43823427 | 10783 | 27: 43863511 | 10783 | 27: 43908553 | 10784 | 27: 43927325 | 10784 | 27: 43987089 |
| 10785 | 27: 43995458 | 10785 | 27: 44200677 | 10787 | 27: 44375041 | 10787 | 27: 44412495 | 10788 | 27: 44543531 |
| 10788 | 27: 44629778 | 10792 | 28: 479125 | 10792 | 28: 504407 | 10793 | 28: 692680 | 10793 | 28: 704706 |
| 10793 | 28: 704958 | 10794 | 28: 1196781 | 10795 | 28: 1230989 | 10795 | 28: 1234367 | 10795 | 28: 1243626 |
| 10795 | 28: 1243641 | 10795 | 28: 1243692 | 10795 | 28: 1320241 | 10796 | 28: 1399889 | 10796 | 28: 1487331 |
| 10796 | 28: 1489641 | 10798 | 28: 1783556 | 10798 | 28: 1818831 | 10798 | 28: 1840156 | 10799 | 28: 1924353 |
| 10799 | 28: 1964866 | 10799 | 28: 2032083 | 10800 | 28: 2144906 | 10800 | 28: 2162175 | 10801 | 28: 2343692 |
| 10802 | 28: 2709799 | 10802 | 28: 2730701 | 10803 | 28: 3272910 | 10803 | 28: 3476063 | 10803 | 28: 3548826 |
| 10803 | 28: 3590983 | 10804 | 28: 3973141 | 10804 | 28: 3973211 | 10804 | 28: 3984834 | 10805 | 28: 4431180 |
| 10805 | 28: 4631548 | 10806 | 28: 4701678 | 10806 | 28: 4907755 | 10807 | 28: 5014037 | 10808 | 28: 5220057 |
| 10808 | 28: 5220146 | 10808 | 28: 5220186 | 10808 | 28: 5245742 | 10808 | 28: 5247877 | 10808 | 28: 5253360 |
| 1081C | 28: 5630191 | 10810 | 28: 5815635 | 10810 | 28: 5833150 | 10811 | 28: 5839078 | 10811 | 28: 5870781 |
| 10811 | 28: 5870881 | 10811 | 28: 5873548 | 10811 | 28: 5873612 | 10811 | 28: 5873655 | 10811 | 28: 5874287 |
| 10811 | 28: 5903348 | 10814 | 28: 6691792 | 10815 | 28: 6866814 | 10815 | 28: 6866847 | 10815 | 28: 6866914 |
| 10816 | 28: 7257360 | 10817 | 28: 7304728 | 10817 | 28: 7321818 | 10817 | 28: 7339237 | 10817 | 28: 7378556 |
| 10817 | 28: 7415736 | 10818 | 28: 7555026 | 10818 | 28: 7760692 | 10819 | 28: 7786515 | 10819 | 28: 7813529 |
| 10820 | 28: 8105292 | 10820 | 28: 8125888 | 10821 | 28: 8201519 | 10821 | 28: 8331969 | 10822 | 28: 8547431 |
| 10823 | 28: 8650652 | 10823 | 28: 8698842 | 10823 | 28: 8707863 | 10824 | 28: 8843955 | 10824 | 28: 9025349 |
| 10825 | 28: 9100351 | 10825 | 28: 9269631 | 10826 | 28: 9336102 | 10826 | 28: 9386021 | 10826 | 28: 9586664 |
| 10828 | 28: 9807554 | 10828 | 28: 9913957 | 10828 | 28: 9914017 | 10828 | 28: 9915219 | 10828 | 28: 9915233 |
| 10830 | 28: 10277520 | 10835 | 28: 12225835 | 10837 | 28: 12371401 | 10838 | 28: 12623784 | 10838 | 28: 12623831 |
| 10838 | 28: 12623855 | 10840 | 28: 13022825 | 10841 | 28: 13252341 | 10843 | 28: 13950890 | 10844 | 28: 14104824 |
| 10844 | 28: 14144978 | 10844 | 28: 14157633 | 10844 | 28: 14181805 | 10844 | 28: 14204540 | 10845 | 28: 14219987 |
| 10845 | 28: 14342309 | 10845 | 28: 14415424 | 10845 | 28: 14415472 | 10846 | 28: 14547089 | 10846 | 28: 14552705 |
| 10847 | 28: 14925580 | 10848 | 28: 14960497 | 10848 | 28: 15003137 | 10849 | 28: 15386201 | 10849 | 28: 15392967 |
| 10850 | 28: 15541195 | 10850 | 28: 15543708 | 10850 | 28: 15562551 | 10851 | 28: 15866947 | 10851 | 28: 15875883 |
| 10851 | 28: 15913989 | 10851 | 28: 15913997 | 10852 | 28: 15914061 | 10852 | 28: 15914091 | 10852 | 28: 15942493 |
| 10852 | 28: 15972055 | 10852 | 28: 16010478 | 10853 | 28: 16039905 | 10853 | 28: 16124447 | 10853 | 28: 16124496 |
| 10854 | 28: 16335973 | 10854 | 28: 16387453 | 10854 | 28: 16407912 | 10855 | 28: 16520465 | 10855 | 28: 16588164 |
| 10855 | 28: 16644285 | 10855 | 28: 16644893 | 10855 | 28: 16648829 | 10856 | 28: 16661175 | 10856 | 28: 16661840 |
| 10856 | 28: 16692080 | 10856 | 28: 16767636 | 10856 | 28: 16773322 | 10856 | 28: 16818843 | 10857 | 28: 16930270 |
| 10858 | 28: 17215906 | 10858 | 28: 17282866 | 10859 | 28: 17285666 | 10859 | 28: 17285711 | 10860 | 28: 17588476 |
| 10861 | 28: 17835910 | 10862 | 28: 18019905 | 10862 | 28: 18019907 | 10862 | 28: 18019913 | 10862 | 28: 18019925 |
| 10863 | 28: 18086730 | 10863 | 28: 18107994 | 10864 | 28: 18485866 | 10866 | 28: 18937069 | 10866 | 28: 19041963 |
| 10866 | 28: 19127485 | 10867 | 28: 19229652 | 10868 | 28: 19534804 | 10868 | 28: 19583995 | 10868 | 28: 19673238 |
| 10869 | 28: 19774362 | 10871 | 28: 20415689 | 10875 | 28: 21574143 | 10878 | 28: 22399855 | 10878 | 28: 22479622 |
| 10879 | 28: 22620494 | 10879 | 28: 22620545 | 10879 | 28: 22620582 | 10880 | 28: 22847773 | 10880 | 28: 23007374 |
| 10880 | 28: 23007384 | 10880 | 28: 23049818 | 10881 | 28: 23173525 | 10881 | 28: 23177219 | 10882 | 28: 23292046 |
| 10882 | 28: 23296757 | 10882 | 28: 23385467 | 10883 | 28: 23385512 | 10885 | 28: 24001450 | 10886 | 28: 24160079 |
| 10886 | 28: 24160811 | 10886 | 28: 24242145 | 10886 | 28: 24253020 | 10888 | 28: 24623959 | 10889 | 28: 24802411 |
| 10889 | 28: 24802550 | 10889 | 28: 24802551 | 10889 | 28: 24885896 | 10890 | 28: 24934653 | 10890 | 28: 25064734 |
| 10890 | 28: 25090839 | 10890 | 28: 25139537 | 10891 | 28: 25385010 | 10891 | 28: 25408853 | 10892 | 28: 25415208 |
| 10892 | 28: 25614547 | 10892 | 28: 25625624 | 10893 | 28: 25625649 | 10893 | 28: 25625667 | 10893 | 28: 25626954 |
| 10893 | 28: 25731593 | 10893 | 28: 25808647 | 10894 | 28: 25947218 | 10894 | 28: 26030383 | 10894 | 28: 26036200 |
| 10895 | 28: 26155221 | 10895 | 28: 26180213 | 10896 | 28: 26181587 | 10896 | 28: 26181622 | 10896 | 28: 26181655 |
| 10896 | 28: 26185280 | 10896 | 28: 26185344 | 10896 | 28: 26185376 | 10896 | 28: 26186082 | 10896 | 28: 26186104 |
| 10896 | 28: 26186116 | 10896 | 28: 26186150 | 10898 | 28: 26504153 | 10898 | 28: 26606263 | 10899 | 28: 26681661 |
| 10899 | 28: 26740880 | 10899 | 28: 26752280 | 10899 | 28: 26814555 | 10900 | 28: 27066599 | 10900 | 28: 27068380 |
| 10901 | 28: 27070779 | 10901 | 28: 27146373 | 10902 | 28: 27317961 | 10903 | 28: 27518059 | 10903 | 28: 27566739 |
| 10904 | 28: 27704605 | 10904 | 28: 27851635 | 10905 | 28: 27937188 | 10905 | 28: 27937338 | 10905 | 28: 27937971 |
| 10906 | 28: 27941915 | 10906 | 28: 27941916 | 10906 | 28: 27941932 | 10906 | 28: 27943776 | 10906 | 28: 27943868 |
| 10906 | 28: 27943901 | 10906 | 28: 27943916 | 10906 | 28: 27948642 | 10906 | 28: 27948643 | 10907 | 28: 27954047 |
| 10907 | 28: 28109673 | 10908 | 28: 28192038 | 10908 | 28: 28290243 | 10909 | 28: 28290255 | 10909 | 28: 28290934 |
| 10909 | 28: 28290955 | 10909 | 28: 28290999 | 10909 | 28: 28313393 | 10910 | 28: 28407860 | 10910 | 28: 28646964 |
| 10910 | 28: 28722550 | 10911 | 28: 28920066 | 10911 | 28: 28920811 | 10911 | 28: 28920813 | 10913 | 28: 29172597 |
| 10913 | 28: 29210804 | 10913 | 28: 29213687 | 10913 | 28: 29236992 | 10913 | 28: 29298781 | 10915 | 28: 29704456 |
| 10915 | 28: 29735862 | 10915 | 28: 29737399 | 10915 | 28: 29776190 | 10916 | 28: 29777653 | 10916 | 28: 29841383 |
| 10916 | 28: 29914673 | 10917 | 28: 30046702 | 10917 | 28: 30226392 | 10918 | 28: 30648480 | 10919 | 28: 30730091 |
| 10919 | 28: 30770433 | 10919 | 28: 30824174 | 10920 | 28: 30869446 | 10920 | 28: 30999414 | 10920 | 28: 31017300 |
| 10920 | 28: 31042068 | 10921 | 28: 31093235 | 10921 | 28: 31386148 | 10922 | 28: 31501902 | 10922 | 28: 31528266 |
| 10922 | 28: 31531949 | 10923 | 28: 31783641 | 10925 | 28: 32102216 | 10925 | 28: 32102950 | 10925 | 28: 32104334 |
| 10925 | 28: 32119157 | 10925 | 28: 32119371 | 10925 | 28: 32122100 | 10925 | 28: 32141403 | 10926 | 28: 32322725 |
| 10927 | 28: 32385967 | 10927 | 28: 32392342 | 10930 | 28: 32904173 | 10930 | 28: 32960277 | 10931 | 28: 32997331 |
| 10931 | 28: 33041333 | 10932 | 28: 33158395 | 10932 | 28: 33186798 | 10932 | 28: 33225867 | 10932 | 28: 33239306 |
| 10933 | 28: 33329500 | 10933 | 28: 33342708 | 10933 | 28: 33368759 | 10934 | 28: 33551708 | 10934 | 28: 33555527 |
| 10934 | 28: 33662006 | 10935 | 28: 33744728 | 10935 | 28: 33822791 | 10935 | 28: 33823084 | 10936 | 28: 34010065 |
| 10936 | 28: 34062194 | 10937 | 28: 34243304 | 10937 | 28: 34254734 | 10937 | 28: 34333090 | 10938 | 28: 34496870 |
| 10938 | 28: 34523399 | 10939 | 28: 34603788 | 10940 | 28: 34778574 | 10940 | 28: 34786048 | 10940 | 28: 34828202 |
| 10941 | 28: 34853555 | 10941 | 28: 34912784 | 10941 | 28: 34993324 | 10942 | 28: 35011293 | 10942 | 28: 35125823 |
| 10942 | 28: 35132199 | 10942 | 28: 35132247 | 10943 | 28: 35222450 | 10943 | 28: 35291319 | 10944 | 28: 35644657 |
| 10944 | 28: 35671648 | 10944 | 28: 35672175 | 10944 | 28: 35680732 | 10944 | 28: 35683234 | 10944 | 28: 35685429 |
| 10945 | 28: 35805891 | 10945 | 28: 35837667 | 10945 | 28: 35923596 | 10946 | 28: 36015548 | 10947 | 28: 36205983 |
| 10948 | 28: 36587691 | 10952 | 28: 37533725 | 10954 | 28: 37721207 | 10960 | 28: 39163650 | 10960 | 28: 39164865 |
| 10960 | 28: 39164902 | 10960 | 28: 39164949 | 10960 | 28: 39167717 | 10960 | 28: 39167797 | 10960 | 28: 39167807 |
| 10960 | 28: 39167808 | 10960 | 28: 39167836 | 10960 | 28: 39167853 | 10961 | 28: 39266326 | 10961 | 28: 39290663 |
| 10961 | 28: 39316523 | 10963 | 28: 39818069 | 10963 | 28: 39868243 | 10966 | 28: 40526877 | 10966 | 28: 40526886 |
| 10966 | 28: 40547129 | 10966 | 28: 40606521 | 10966 | 28: 40609071 | 10967 | 28: 40613418 | 10967 | 28: 40641661 |
| 10967 | 28: 40644608 | 10967 | 28: 40647540 | 10967 | 28: 40648502 | 10967 | 28: 40649183 | 10967 | 28: 40650128 |
| 10967 | 28: 40650866 | 10968 | 28: 40651548 | 10968 | 28: 40652228 | 10968 | 28: 40653623 | 10968 | 28: 40654155 |
| 10968 | 28: 40654919 | 10968 | 28: 40655651 | 10968 | 28: 40656367 | 10968 | 28: 40658031 | 10968 | 28: 40659095 |
| 10969 | 28: 40679353 | 10969 | 28: 40683789 | 10969 | 28: 40684338 | 10969 | 28: 40685176 | 10969 | 28: 40685911 |
| 10969 | 28: 40687468 | 10969 | 28: 40688010 | 10969 | 28: 40688917 | 10969 | 28: 40690412 | 10969 | 28: 40691166 |
| 10970 | 28: 40692798 | 10970 | 28: 40693384 | 10970 | 28: 40694350 | 10970 | 28: 40695394 | 10970 | 28: 40697087 |
| 10970 | 28: 40698875 | 10970 | 28: 40700189 | 10970 | 28: 40701565 | 10970 | 28: 40707053 | 10970 | 28: 40707560 |
| 10971 | 28: 40708330 | 10971 | 28: 40709943 | 10971 | 28: 40711617 | 10971 | 28: 40714408 | 10971 | 28: 40716186 |
| 10971 | 28: 40717895 | 10971 | 28: 40718746 | 10971 | 28: 40719687 | 10971 | 28: 40720636 | 10971 | 28: 40721303 |
| 10972 | 28: 40722936 | 10972 | 28: 40723874 | 10972 | 28: 40724503 | 10972 | 28: 40725696 | 10972 | 28: 40726977 |
| 10973 | 28: 40857491 | 10974 | 28: 40977297 | 10974 | 28: 41002895 | 10974 | 28: 41077744 | 10975 | 28: 41244211 |
| 10975 | 28: 41283427 | 10975 | 28: 41333995 | 10977 | 28: 41565674 | 10978 | 28: 41666097 | 10978 | 28: 41666186 |
| 10978 | 28: 41724320 | 10978 | 28: 41728249 | 10978 | 28: 41731568 | 10978 | 28: 41757290 | 10979 | 28: 41885934 |
| 10979 | 28: 41913803 | 10980 | 28: 41952391 | 10980 | 28: 41980728 | 10980 | 28: 41986845 | 10980 | 28: 41989663 |
| 10980 | 28: 42038658 | 10981 | 28: 42092356 | 10982 | 28: 42424892 | 10983 | 28: 42588414 | 10983 | 28: 42648031 |
| 10984 | 28: 42816073 | 10984 | 28: 42822333 | 10984 | 28: 42894677 | 10985 | 28: 43061595 | 10985 | 28: 43097491 |
| 10985 | 28: 43111417 | 10985 | 28: 43111489 | 10985 | 28: 43111570 | 10985 | 28: 43112699 | 10986 | 28: 43112752 |
| 10986 | 28: 43112807 | 10986 | 28: 43112817 | 10986 | 28: 43112858 | 10986 | 28: 43140781 | 10987 | 28: 43230125 |
| 10987 | 28: 43258213 | 10988 | 28: 43420442 | 10988 | 28: 43482124 | 10988 | 28: 43501640 | 10988 | 28: 43538406 |
| 10989 | 28: 43579200 | 10989 | 28: 43651140 | 10990 | 28: 44028372 | 10990 | 28: 44030407 | 10990 | 28: 44031081 |
| 10990 | 28: 44038721 | 10991 | 28: 44039225 | 10991 | 28: 44039304 | 10991 | 28: 44143344 | 10991 | 28: 44157666 |
| 10992 | 28: 44264765 | 10993 | 28: 44453518 | 10994 | 28: 44687207 | 10994 | 28: 44852965 | 10996 | 28: 45172059 |
| 10997 | 28: 45281713 | 10997 | 28: 45314101 | 10998 | 28: 45416826 | 10998 | 28: 45476974 | 10999 | 28: 45579104 |
| 11000 | 28: 45713679 | 11000 | 28: 45771532 | 11000 | 28: 45847568 | 11001 | 29: 612126 | 11001 | 29: 699249 |
| 11002 | 29: 699337 | 11002 | 29: 699732 | 11002 | 29: 699767 | 11002 | 29: 750365 | 11004 | 29: 1219671 |
| 11004 | 29: 1273559 | 11004 | 29: 1315459 | 11005 | 29: 1429261 | 11005 | 29: 1499682 | 11005 | 29: 1512567 |
| 11006 | 29: 1512626 | 11007 | 29: 1772440 | 11008 | 29: 2136150 | 11009 | 29: 2325537 | 11009 | 29: 2380491 |
| 11011 | 29: 2594653 | 11011 | 29: 2620380 | 11013 | 29: 2994930 | 11014 | 29: 3175555 | 11015 | 29: 3406656 |
| 11016 | 29: 3717232 | 11019 | 29: 4246237 | 11019 | 29: 4406319 | 11020 | 29: 4643160 | 11022 | 29: 5107639 |
| 11025 | 29: 6283155 | 11026 | 29: 6283208 | 11027 | 29: 6779650 | 11028 | 29: 7101326 | 11029 | 29: 7255100 |
| 11029 | 29: 7426428 | 11033 | 29: 8601758 | 11034 | 29: 8772433 | 11035 | 29: 8793187 | 11035 | 29: 8821458 |
| 11035 | 29: 8883493 | 11035 | 29: 8926820 | 11036 | 29: 8998775 | 11036 | 29: 9022834 | 11036 | 29: 9022891 |
| 11036 | 29: 9102521 | 11037 | 29: 9365821 | 11038 | 29: 9490114 | 11040 | 29: 9846120 | 11040 | 29: 9882247 |
| 11042 | 29: 10340072 | 11044 | 29: 10979545 | 11046 | 29: 11204386 | 11046 | 29: 11241638 | 11046 | 29: 11260132 |
| 11046 | 29: 11341144 | 11047 | 29: 11709562 | 11048 | 29: 11839933 | 11048 | 29: 11891707 | 11049 | 29: 11952953 |
| 11049 | 29: 11977550 | 11049 | 29: 12018235 | 11049 | 29: 12084416 | 11050 | 29: 12114717 | 11050 | 29: 12138482 |
| 11050 | 29: 12182249 | 11051 | 29: 12394797 | 11051 | 29: 12538136 | 11051 | 29: 12589589 | 11052 | 29: 12619588 |
| 11052 | 29: 12622700 | 11054 | 29: 12987411 | 11055 | 29: 13574420 | 11055 | 29: 13574513 | 11056 | 29: 13821500 |
| 11058 | 29: 14373492 | 11058 | 29: 14374640 | 11059 | 29: 14741850 | 11059 | 29: 14741861 | 11059 | 29: 14741873 |
| 11059 | 29: 14741887 | 11060 | 29: 14741921 | 11060 | 29: 14900262 | 11061 | 29: 15134872 | 11062 | 29: 15304699 |
| 11062 | 29: 15395863 | 11064 | 29: 15811607 | 11066 | 29: 16464456 | 11066 | 29: 16485296 | 11069 | 29: 16964102 |
| 11069 | 29: 16992973 | 11069 | 29: 17055985 | 11070 | 29: 17207473 | 11070 | 29: 17442223 | 11071 | 29: 17505934 |
| 11071 | 29: 17540650 | 11071 | 29: 17700379 | 11073 | 29: 18393448 | 11073 | 29: 18612768 | 11078 | 29: 20127489 |
| 11079 | 29: 20506894 | 11079 | 29: 20644568 | 11080 | 29: 20651157 | 11080 | 29: 20673987 | 11080 | 29: 20674048 |
| 11082 | 29: 21228889 | 11082 | 29: 21315170 | 11083 | 29: 21392121 | 11083 | 29: 21392184 | 11083 | 29: 21392189 |
| 11084 | 29: 21600071 | 11086 | 29: 22072294 | 11086 | 29: 22180877 | 11086 | 29: 22297264 | 11086 | 29: 22344163 |
| 11088 | 29: 22760861 | 11088 | 29: 22836272 | 11090 | 29: 23318559 | 11090 | 29: 23357907 | 11091 | 29: 23560620 |
| 11092 | 29: 23583982 | 11092 | 29: 23628287 | 11092 | 29: 23661992 | 11092 | 29: 23731784 | 11093 | 29: 23850587 |
| 11093 | 29: 23937388 | 11094 | 29: 24158412 | 11095 | 29: 24258248 | 11095 | 29: 24330084 | 11095 | 29: 24354233 |
| 11095 | 29: 24362811 | 11095 | 29: 24490616 | 11096 | 29: 24840874 | 11097 | 29: 25015460 | 11098 | 29: 25071999 |
| 11098 | 29: 25205531 | 11099 | 29: 25222326 | 11099 | 29: 25305438 | 11099 | 29: 25371765 | 11099 | 29: 25438251 |
| 11101 | 29: 25903150 | 11101 | 29: 25908734 | 11101 | 29: 26070069 | 11102 | 29: 26105651 | 11103 | 29: 26549594 |
| 11104 | 29: 26946250 | 11104 | 29: 26946336 | 11104 | 29: 26946383 | 11105 | 29: 26947201 | 11105 | 29: 26947224 |
| 11105 | 29: 26962161 | 11106 | 29: 27537716 | 11106 | 29: 27537760 | 11106 | 29: 27538148 | 11107 | 29: 27595444 |
| 11107 | 29: 27728132 | 11108 | 29: 27790794 | 11108 | 29: 27808076 | 11108 | 29: 27808486 | 11108 | 29: 27823998 |
| 11108 | 29: 27824016 | 11108 | 29: 27824039 | 11109 | 29: 27824126 | 11109 | 29: 28026650 | 11109 | 29: 28100144 |
| 11110 | 29: 28218734 | 11110 | 29: 28226254 | 11110 | 29: 28228229 | 11110 | 29: 28251283 | 111111 | 29: 28278699 |
| 111117 | 29: 28370448 | 111118 | 29: 28430575 | 11112 | 29: 28519839 | 11112 | 29: 28574787 | 11112 | 29: 28655873 |
| 11112 | 29: 28715263 | 11113 | 29: 28815692 | 11113 | 29: 28826292 | 11115 | 29: 29397513 | 11116 | 29: 29397515 |
| 11116 | 29: 29461701 | 11116 | 29: 29472010 | 11116 | 29: 29555287 | 11117 | 29: 29629619 | 11117 | 29: 29649318 |
| 11117 | 29: 29653990 | 11117 | 29: 29665600 | 11117 | 29: 29676297 | 11117 | 29: 29682536 | 11117 | 29: 29690811 |
| 11118 | 29: 29755215 | 11118 | 29: 29755216 | 11118 | 29: 29755261 | 11118 | 29: 29755320 | 11118 | 29: 29756679 |
| 11118 | 29: 29756692 | 11119 | 29: 29928478 | 11119 | 29: 29928524 | 11119 | 29: 29928553 | 11119 | 29: 29928591 |
| 11120 | 29: 29928636 | 11120 | 29: 29928675 | 11120 | 29: 29928677 | 11121 | 29: 30201851 | 11121 | 29: 30294652 |
| 11123 | 29: 30759911 | 11124 | 29: 30759984 | 11125 | 29: 31124828 | 11126 | 29: 31355454 | 11126 | 29: 31355522 |
| 11127 | 29: 31455230 | 11127 | 29: 31488540 | 11128 | 29: 31676141 | 11128 | 29: 31801460 | 11128 | 29: 31801542 |
| 11128 | 29: 31845002 | 11128 | 29: 31845960 | 11130 | 29: 32253717 | 11131 | 29: 32397724 | 11131 | 29: 32544942 |
| 11131 | 29: 32611173 | 11132 | 29: 32693113 | 11132 | 29: 32753972 | 11132 | 29: 32754754 | 11132 | 29: 32764982 |
| 11132 | 29: 32765663 | 11132 | 29: 32766346 | 11133 | 29: 32842956 | 11134 | 29: 33120665 | 11135 | 29: 33560766 |
| 11136 | 29: 33704537 | 11137 | 29: 33824948 | 11139 | 29: 34433856 | 11141 | 29: 34688201 | 11142 | 29: 34751704 |
| 11142 | 29: 34751748 | 11143 | 29: 34985663 | 11144 | 29: 35373548 | 11144 | 29: 35393862 | 11144 | 29: 35393931 |
| 11145 | 29: 35506238 | 11146 | 29: 35663595 | 11146 | 29: 35697578 | 11147 | 29: 35864204 | 11147 | 29: 35918743 |
| 11148 | 29: 36106383 | 11148 | 29: 36108259 | 11149 | 29: 36148048 | 11149 | 29: 36206784 | 11149 | 29: 36207683 |
| 11150 | 29: 36388797 | 11151 | 29: 36481048 | 11152 | 29: 36766544 | 11152 | 29: 36775708 | 11152 | 29: 36800395 |
| 11152 | 29: 36811950 | 11152 | 29: 36813596 | 11153 | 29: 36934958 | 11153 | 29: 36975121 | 11154 | 29: 37168472 |
| 11154 | 29: 37221849 | 11155 | 29: 37332886 | 11155 | 29: 37332893 | 11155 | 29: 37342276 | 11155 | 29: 37411101 |
| 11156 | 29: 37440660 | 11156 | 29: 37458594 | 11156 | 29: 37523548 | 11160 | 29: 39536492 | 11161 | 29: 39891282 |
| 11162 | 29: 39975025 | 11163 | 29: 40151594 | 11163 | 29: 40297458 | 11163 | 29: 40314843 | 11164 | 29: 40481400 |
| 11164 | 29: 40518208 | 11164 | 29: 40595649 | 11165 | 29: 40805372 | 11165 | 29: 40806225 | 11166 | 29: 40851566 |
| 11167 | 29: 40878959 | 11167 | 29: 40879481 | 11167 | 29: 40919327 | 11167 | 29: 41004847 | 11168 | 29: 41038278 |
| 11168 | 29: 41142315 | 11168 | 29: 41142793 | 11169 | 29: 41279012 | 11169 | 29: 41338346 | 11169 | 29: 41342454 |
| 11170 | 29: 41493354 | 11171 | 29: 41557548 | 11171 | 29: 41564691 | 11171 | 29: 41642963 | 11171 | 29: 41687163 |
| 11171 | 29: 41749646 | 11172 | 29: 41785049 | 11172 | 29: 41789280 | 11172 | 29: 41792341 | 11172 | 29: 41823721 |
| 11172 | 29: 41947165 | 11173 | 29: 41976753 | 11173 | 29: 42018005 | 11173 | 29: 42022273 | 11173 | 29: 42031453 |
| 11175 | 29: 42391692 | 11175 | 29: 42509147 | 11175 | 29: 42513501 | 11176 | 29: 42528204 | 11176 | 29: 42615699 |
| 11176 | 29: 42635388 | 11176 | 29: 42642354 | 11176 | 29: 42657038 | 11176 | 29: 42659255 | 11176 | 29: 42659448 |
| 11177 | 29: 42844516 | 11178 | 29: 42982094 | 11178 | 29: 43052218 | 11178 | 29: 43090186 | 11179 | 29: 43206957 |
| 11179 | 29: 43207132 | 11179 | 29: 43207525 | 11179 | 29: 43207573 | 11179 | 29: 43207753 | 11179 | 29: 43210991 |
| 11179 | 29: 43218240 | 11180 | 29: 43282578 | 11180 | 29: 43391770 | 11181 | 29: 43495617 | 11181 | 29: 43524321 |
| 11182 | 29: 43620118 | 11182 | 29: 43622532 | 11182 | 29: 43627877 | 11182 | 29: 43631768 | 11183 | 29: 43704875 |
| 11183 | 29: 43750816 | 11183 | 29: 43750820 | 11183 | 29: 43814690 | 11183 | 29: 43827364 | 11183 | 29: 43880340 |
| 11184 | 29: 43883371 | 11184 | 29: 43909067 | 11184 | 29: 43968492 | 11184 | 29: 43970000 | 11184 | 29: 43981948 |
| 11184 | 29: 43982210 | 11186 | 29: 44265806 | 11186 | 29: 44285086 | 11186 | 29: 44314568 | 11187 | 29: 44423551 |
| 11188 | 29: 44484826 | 11188 | 29: 44552650 | 11188 | 29: 44627679 | 11189 | 29: 44770084 | 11189 | 29: 44991893 |
| 11190 | 29: 45224759 | 11191 | 29: 45378882 | 11191 | 29: 45460407 | 11191 | 29: 45538531 | 11192 | 29: 45566486 |
| 11192 | 29: 45574701 | 11192 | 29: 45601782 | 11192 | 29: 45749221 | 11193 | 29: 45756539 | 11193 | 29: 45875877 |
| 11193 | 29: 45897665 | 11193 | 29: 45951010 | 11194 | 29: 45996529 | 11194 | 29: 46069944 | 11195 | 29: 46268948 |
| 11195 | 29: 46344185 | 11196 | 29: 46344338 | 11199 | 29: 47058184 | 11199 | 29: 47089599 | 11199 | 29: 47171895 |
| 11200 | 29: 47412530 | 11200 | 29: 47512321 | 11201 | 29: 47628422 | 11203 | 29: 47965964 | 11203 | 29: 48004279 |
| 11204 | 29: 48072601 | 11205 | 29: 48380729 | 11205 | 29: 48412845 | 11205 | 29: 48499387 | 11206 | 29: 48750418 |
| 11207 | 29: 48886024 | 11207 | 29: 48936775 | 11210 | 29: 49277470 | 11211 | 29: 49503212 | 11211 | 29: 49583754 |
| 11211 | 29: 49601539 | 11211 | 29: 49645960 | 11211 | 29: 49646022 | 11212 | 29: 49706299 | 11213 | 29: 50015783 |
| 11214 | 29: 50279949 | 11214 | 29: 50279990 | 11214 | 29: 50326170 | 11215 | 29: 50499209 | 11215 | 29: 50566734 |
| 11215 | 29: 50572585 | 11215 | 29: 50573514 | 11215 | 29: 50595441 | 11216 | 29: 50698048 | 11216 | 29: 50698051 |
| 11216 | 29: 50698125 | 11216 | 29: 50699876 | 11216 | 29: 50700624 | 11217 | 29: 50702620 | 11217 | 29: 50777521 |
| 11217 | 29: 50809985 | 11217 | 29: 50901915 | 11217 | 29: 50902670; | | | | |

and
the sites of the third marker panel are shown in the following table:

| NO. | Position | NO. | Position | NO. | Position | NO. | Position | NO. | Position |
|---|---|---|---|---|---|---|---|---|---|
| 188 | 1:395886 | 339 | 1:703722 | 420 | 1:906974 | 1171 | 1:2996340 | 1536 | 1:4057672 |
| 2248 | 1:589653 | 2403 | 1:626739 | 3852 | 1:985197 | 4392 | 1:1131760 | 4969 | 1:1264288 |
| 6050 | 1:149646 | 6468 | 1:156749 | 6762 | 2:537453 | 6787 | 2:5824469 | 6980 | 2:1042500 |
| 7049 | 2:128028 | 7090 | 2:138220 | 7681 | 2:269491 | 8255 | 2:4182328 | 8310 | 2:4316452 |
| 8401 | 2:457305 | 1090 | 2:110396 | 1119 | 2:117962 | 1166 | 2:1282694 | 1277 | 3:1267167 |
| 1313 | 3:211447 | 1403 | 3:395491 | 1406 | 3:402555 | 1410 | 3:4160522 | 1442 | 3:4954803 |
| 1453 | 3:518176 | 1475 | 3:578965 | 1529 | 3:734325 | 1573 | 3:8499077 | 1615 | 3:9391427 |
| 1628 | 3:975958 | 1723 | 3:115822 | 1798 | 4:109063 | 1818 | 4:1621515 | 1838 | 4:2065221 |
| 1897 | 4:367420 | 1915 | 4:414001 | 1971 | 4:574214 | 2019 | 4:7061032 | 2025 | 4:7253292 |
| 2047 | 4:779178 | 2197 | 4:109990 | 2288 | 5:765657 | 2371 | 5:2767099 | 2388 | 5:3169176 |
| 2505 | 5:629338 | 2520 | 5:666939 | 2543 | 5:719145 | 2568 | 5:7804644 | 2622 | 5:8792394 |
| 2671 | 5:976312 | 2750 | 5:112616 | 2783 | 5:118152 | 2851 | 6:1273217 | 2895 | 6:2221084 |
| 2919 | 6:281131 | 2919 | 6:281318 | 3226 | 6:429944 | 3250 | 6:4536462 | 3309 | 6:5955160 |
| 3311 | 6:598689 | 3355 | 6:728525 | 3507 | 6:109291 | 3527 | 6:1134849 | 3591 | 7:7248738 |
| 3605 | 7:121749 | 3634 | 7:172230 | 3671 | 7:248614 | 3790 | 7:5323337 | 3903 | 7:7930430 |
| 3904 | 7:793814 | 3910 | 7:805361 | 3961 | 7:918583 | 3970 | 7:9452918 | 4012 | 7:1009550 |
| 4031 | 7:105797 | 4055 | 8:168853 | 4167 | 8:287478 | 4284 | 8:5958533 | 4394 | 8:8752304 |
| 4449 | 8:101431 | 4466 | 8:104418 | 4553 | 9:142246 | 4608 | 9:2777847 | 4704 | 9:5170207 |
| 4729 | 9:585114 | 4832 | 9:822086 | 4908 | 9:970297 | 4966 | 10:358243 | 5021 | 10:146014 |
| 5033 | 10:16803 | 5143 | 10:44059 | 5146 | 10:44872 | 5192 | 10:555395 | 5212 | 10:614624 |
| 5294 | 10:81223 | 5300 | 10:82399 | 5307 | 10:84068 | 5367 | 10:971526 | 5448 | 11:940047 |
| 5509 | 11:24487 | 5558 | 11:36861 | 5596 | 11:46544 | 5660 | 11:620426 | 5663 | 11:627814 |
| 5678 | 11:66365 | 5735 | 11:77817 | 5818 | 11:95543 | 5851 | 11:102567 | 5855 | 11:103007 |
| 5918 | 12:11793 | 5977 | 12:25646 | 5999 | 12:31268 | 6090 | 12:578674 | 6099 | 12:609977 |
| 6124 | 12:68116 | 6155 | 12:75672 | 6156 | 12:76081 | 6217 | 13:207965 | 6317 | 13:253355 |
| 6422 | 13:47037 | 6513 | 13:68527 | 6542 | 13:74664 | 6584 | 13:826673 | 6638 | 14:911668 |
| 6643 | 14:10036 | 6757 | 14:26068 | 6840 | 14:46226 | 6866 | 14:534793 | 6896 | 14:602989 |
| 6969 | 14:77723 | 6977 | 14:79615 | 7089 | 15:20899 | 7148 | 15:329126 | 7170 | 15:374722 |
| 7170 | 15:37547 | 7194 | 15:42887 | 7229 | 15:49801 | 7231 | 15:506934 | 7297 | 15:638330 |
| 7359 | 15:77950 | 7425 | 16:92596 | 7433 | 16:11622 | 7440 | 16:130724 | 7460 | 16:191974 |
| 7675 | 16:71035 | 7696 | 16:75329 | 7727 | 17:10232 | 7766 | 17:913211 | 7781 | 17:127266 |
| 7801 | 17:17314 | 7846 | 17:29524 | 7847 | 17:29949 | 7853 | 17:314780 | 7929 | 17:504856 |
| 7946 | 17:54264 | 7998 | 17:64965 | 8050 | 18:18019 | 8074 | 18:674782 | 8150 | 18:233514 |
| 8157 | 18:24699 | 8166 | 18:26757 | 8251 | 18:46459 | 8268 | 18:485458 | 8283 | 18:521782 |
| 8345 | 18:64214 | 8371 | 19:44783 | 8389 | 19:82749 | 8403 | 19:108612 | 8423 | 19:150178 |
| 8432 | 19:16900 | 8553 | 19:38864 | 8558 | 19:40043 | 8579 | 19:441735 | 8624 | 19:522932 |
| 8636 | 19:54554 | 8673 | 19:59907 | 8708 | 20:23863 | 8795 | 20:178473 | 8854 | 20:309114 |
| 8917 | 20:46043 | 8962 | 20:58379 | 8983 | 20:63586 | 9034 | 21:304067 | 9114 | 21:208094 |
| 9134 | 21:26120 | 9139 | 21:27418 | 9192 | 21:39994 | 9205 | 21:431339 | 9241 | 21:516923 |
| 9288 | 21:60444 | 9301 | 21:63550 | 9383 | 22:11000 | 9386 | 22:117189 | 9424 | 22:209163 |
| 9428 | 22:21678 | 9431 | 22:22468 | 9440 | 22:25488 | 9546 | 22:505892 | 9559 | 22:534773 |
| 9573 | 22:55883 | 9635 | 23:73186 | 9740 | 23:27495 | 9815 | 23:409936 | 9820 | 23:420218 |
| 9868 | 23:51029 | 9883 | 24:16171 | 9903 | 24:61255 | 9928 | 24:104971 | 9946 | 24:151419 |
| 9970 | 24:21378 | 1006 | 24:44195 | 1011 | 24:55950 | 1017 | 25:313056 | 1023 | 25:145932 |
| 1025 | 25:19902 | 1028 | 25:27084 | 1028 | 25:27654 | 1040 | 26:819240 | 1042 | 26:131947 |
| 1052 | 26:37900 | 1053 | 26:39609 | 1057 | 26:49826 | 1062 | 27:920491 | 1063 | 27:117362 |
| 1064 | 27:13894 | 1065 | 27:16084 | 1067 | 27:19194 | 1068 | 27:224103 | 1074 | 27:357616 |
| 1075 | 27:37824 | 1077 | 27:41177 | 1081 | 28:58742 | 1085 | 28:159724 | 1087 | 28:203712 |
| 1093 | 28:33248 | 1094 | 28:35132 | 1099 | 28:43879 | 1103 | 29:910465 | 1104 | 29:111981 |
| 1111 | 29:28278 | 1111 | 29:28684 | 1118 | 29:44090 | 1121 | 29:508581 | | |

and
the sites of the fourth marker panel are shown in the following table:

| NO. | Position | NO. | Position | NO. | Position | NO. | Position | NO | Position |
|---|---|---|---|---|---|---|---|---|---|
| 41 | 1:1299095 | 42 | 1:1301411 | 43 | 1:1302242 | 72 | 1:1675530 | 92 | 1:2080108 |
| 105 | 1:2256758 | 163 | 1:3595729 | 167 | 1:3620513 | 211 | 1:4298693 | 229 | 1:4729108 |
| 234 | 1:4821451 | 287 | 1:6049970 | 288 | 1:6052470 | 289 | 1:6054551 | 290 | 1:6056604 |
| 291 | 1:6061246 | 373 | 1:7795006 | 388 | 1:8249174 | 413 | 1:8847132 | 417 | 1:8982763 |
| 421 | 1:9088695 | 423 | 1:9118340 | 508 | 1:10838284 | 509 | 1:10876705 | 536 | 1:11604750 |
| 538 | 1:11752900 | 546 | 1:12052406 | 553 | 1:12362501 | 567 | 1:12653822 | 629 | 1:14114440 |
| 630 | 1:14176114 | 721 | 1:16635130 | 728 | 1:16922627 | 747 | 1:17462003 | 756 | 1:17835385 |
| 763 | 1:18110024 | 785 | 1:18793633 | 806 | 1:19543445 | 813 | 1:19664427 | 930 | 1:22893609 |
| 948 | 1:23486726 | 949 | 1:23493262 | 969 | 1:24006741 | 984 | 1:24500834 | 996 | 1:24941722 |
| 1001 | 1:25068708 | 1025 | 1:25577980 | 1129 | 1:29025677 | 1131 | 1:29040742 | 1225 | 1:31664947 |
| 1237 | 1:32296642 | 1256 | 1:32911987 | 1277 | 1:33660199 | 1286 | 1:33837966 | 1287 | 1:33843390 |
| 1288 | 1:33853447 | 1447 | 1:37890316 | 1452 | 1:38007855 | 1516 | 1:39993737 | 1545 | 1:40753557 |
| 1577 | 1:41764542 | 1622 | 1:42618603 | 1693 | 1:44281769 | 1695 | 1:44303304 | 1702 | 1:44457020 |
| 1705 | 1:44558674 | 1834 | 1:47759114 | 1879 | 1:49422581 | 1896 | 1:49969737 | 2024 | 1:53619617 |
| 2025 | 1:53623176 | 2026 | 1:53624697 | 2027 | 1:53629301 | 2028 | 1:53630113 | 2050 | 1:54016006 |
| 2075 | 1:54444238 | 2084 | 1:54690665 | 2116 | 1:55714510 | 2139 | 1:56372440 | 2335 | 1:61145631 |
| 2343 | 1:61319735 | 2362 | 1:61796684 | 2383 | 1:62427513 | 2384 | 1:62433558 | 2430 | 1:63221728 |
| 2462 | 1:64193131 | 2467 | 1:64262857 | 2472 | 1:64316563 | 2499 | 1:64907342 | 2526 | 1:65509479 |
| 2543 | 1:66070553 | 2602 | 1:67691264 | 2624 | 1:68339701 | 2675 | 1:69057373 | 2701 | 1:69645645 |
| 2739 | 1:70443699 | 2766 | 1:70688109 | 2800 | 1:71512292 | 2916 | 1:74460838 | 2934 | 1:74972802 |
| 2936 | 1:74999820 | 2946 | 1:75290882 | 2981 | 1:76075161 | 3007 | 1:76874012 | 3018 | 1:77236634 |
| 3022 | 1:77276581 | 3039 | 1:77623057 | 3041 | 1:77668859 | 3043 | 1:77697633 | 3046 | 1:77710839 |
| 3047 | 1:77723763 | 3060 | 1:78064459 | 3068 | 1:78315800 | 3079 | 1:78505641 | 3100 | 1:79138567 |
| 3115 | 1:79395243 | 3125 | 1:79597067 | 3165 | 1:80126772 | 3190 | 1:80929167 | 3251 | 1:82240750 |
| 3261 | 1:82501216 | 3275 | 1:82703570 | 3311 | 1:83985563 | 3319 | 1:84218035 | 3324 | 1:84478357 |
| 3325 | 1:84493668 | 3336 | 1:85023039 | 3352 | 1:85456698 | 3354 | 1:85501954 | 3382 | 1:86229426 |
| 3407 | 1:86980692 | 3460 | 1:88794416 | 3464 | 1:88824366 | 3545 | 1:90506530 | 3610 | 1:92190821 |
| 3612 | 1:92200875 | 3624 | 1:92563287 | 3638 | 1:93096031 | 3652 | 1:93351663 | 3654 | 1:93372952 |
| 3698 | 1:94573894 | 3724 | 1:95244942 | 3763 | 1:96215196 | 3768 | 1:96305965 | 3790 | 1:96826926 |
| 3791 | 1:96855345 | 3796 | 1:96968145 | 3806 | 1:97319365 | 3829 | 1:97847508 | 3860 | 1:98721777 |
| 3881 | 1:99293036 | 3929 | 1:100616517 | 3939 | 1:100987282 | 4172 | 1:108128123 | 4199 | 1:108777437 |
| 4225 | 1:109419276 | 4227 | 1:109445956 | 4261 | 1:110075319 | 4281 | 1:110389266 | 4305 | 1:111001754 |
| 4330 | 1:111772744 | 4338 | 1:111960165 | 4374 | 1:112577975 | 4386 | 1:112928362 | 4408 | 1:113414092 |
| 4421 | 1:113728857 | 4466 | 1:114828912 | 4467 | 1:114837165 | 4479 | 1:115162087 | 4480 | 1:115169020 |
| 4482 | 1:115183751 | 4521 | 1:116624187 | 4523 | 1:116668988 | 4524 | 1:116683980 | 4567 | 1:117562616 |
| 4604 | 1:118172592 | 4606 | 1:118199028 | 4607 | 1:118200955 | 4660 | 1:119155291 | 4714 | 1:120407128 |
| 4767 | 1:121845822 | 4796 | 1:122567824 | 4957 | 1:126246695 | 4964 | 1:126332119 | 5053 | 1:128692434 |
| 5062 | 1:128825902 | 5078 | 1:129263274 | 5234 | 1:132164555 | 5244 | 1:132550146 | 5263 | 1:133371805 |
| 5292 | 1:134238188 | 5295 | 1:134324146 | 5308 | 1:134489385 | 5335 | 1:135158239 | 5350 | 1:135504827 |
| 5358 | 1:135602604 | 5392 | 1:135798830 | 5450 | 1:137640097 | 5470 | 1:138610452 | 5474 | 1:138652323 |
| 5496 | 1:139117138 | 5556 | 1:140791857 | 5596 | 1:141481667 | 5678 | 1:142851690 | 5679 | 1:142863118 |
| 5697 | 1:143175205 | 5769 | 1:144589518 | 5777 | 1:144761021 | 5823 | 1:145661981 | 5912 | 1:147299699 |
| 5924 | 1:147514403 | 5943 | 1:147887268 | 5993 | 1:148725405 | 6105 | 1:150672403 | 6192 | 1:152095555 |
| 6198 | 1:152196690 | 6272 | 1:153106216 | 6277 | 1:153197360 | 6309 | 1:153828547 | 6340 | 1:154314444 |
| 6357 | 1:154855892 | 6360 | 1:154863975 | 6511 | 1:157806548 | 6568 | 2:1106317 | 6575 | 2:1169877 |
| 6578 | 2:1187683 | 6617 | 2:1860169 | 6621 | 2:1971521 | 6640 | 2:2590948 | 6666 | 2:3510323 |
| 6673 | 2:3709938 | 6718 | 2:4623152 | 6790 | 2:5906646 | 6800 | 2:6055644 | 6971 | 2:10245713 |
| 6979 | 2:10421191 | 7005 | 2:11387282 | 7060 | 2:13168802 | 7064 | 2:13243953 | 7176 | 2:16006718 |
| 7184 | 2:16189620 | 7214 | 2:16976649 | 7240 | 2:17639779 | 7274 | 2:18268568 | 7332 | 2:19056221 |
| 7365 | 2:19833213 | 7582 | 2:24849703 | 7630 | 2:25885564 | 7655 | 2:26463668 | 7685 | 2:27015449 |
| 7704 | 2:27459813 | 7705 | 2:27463924 | 7708 | 2:27525591 | 7760 | 2:28545094 | 7777 | 2:28922483 |
| 7807 | 2:29732106 | 7811 | 2:29813146 | 7889 | 2:31637856 | 7906 | 2:32169593 | 8046 | 2:36375875 |
| 8097 | 2:38039323 | 8098 | 2:38039898 | 8099 | 2:38041017 | 8100 | 2:38042315 | 8116 | 2:38297286 |
| 8120 | 2:38435225 | 8134 | 2:38750534 | 8153 | 2:39280801 | 8169 | 2:39667440 | 8177 | 2:39982069 |
| 8197 | 2:40615263 | 8225 | 2:41307524 | 8262 | 2:42027595 | 8270 | 2:42236387 | 8280 | 2:42439089 |
| 8281 | 2:42440990 | 8298 | 2:42711951 | 8329 | 2:43789161 | 8389 | 2:45494226 | 8399 | 2:45665663 |
| 8419 | 2:46116917 | 8473 | 2:47217427 | 8516 | 2:48495171 | 8517 | 2:48499243 | 8518 | 2:48502118 |
| 8519 | 2:48508248 | 8520 | 2:48511801 | 8560 | 2:49760042 | 8561 | 2:49760735 | 8611 | 2:51218510 |
| 8704 | 2:53518329 | 8730 | 2:54413562 | 8742 | 2:54672099 | 8762 | 2:55031934 | 8764 | 2:55069900 |
| 8765 | 2:55071242 | 8876 | 2:58381419 | 8884 | 2:58588299 | 8913 | 2:59316972 | 8960 | 2:60576956 |
| 8969 | 2:60779135 | 9031 | 2:62457322 | 9119 | 2:64653571 | 9135 | 2:64967881 | 9147 | 2:65185518 |
| 9201 | 2:66960608 | 9235 | 2:68024571 | 9384 | 2:72399848 | 9389 | 2:72514078 | 9418 | 2:73276155 |
| 9479 | 2:74974797 | 9480 | 2:74985065 | 9502 | 2:75739779 | 9535 | 2:76454912 | 9626 | 2:78834490 |
| 9641 | 2:79036468 | 9657 | 2:79510060 | 9681 | 2:80059990 | 9698 | 2:80586285 | 9747 | 2:83003800 |
| 9751 | 2:83109314 | 9755 | 2:83267596 | 9786 | 2:83877880 | 9795 | 2:84259043 | 9799 | 2:84375486 |
| 9832 | 2:85066770 | 9833 | 2:85070174 | 9851 | 2:85646050 | 9887 | 2:86425660 | 9898 | 2:86617347 |
| 9914 | 2:87174608 | 9921 | 2:87410708 | 9941 | 2:88035286 | 9972 | 2:88739478 | 10037 | 2:89511031 |
| 10038 | 2:89515068 | 10039 | 2:89518986 | 10040 | 2:89521529 | 10041 | 2:89523615 | 10076 | 2:90510385 |
| 10078 | 2:90533958 | 10114 | 2:91721645 | 10127 | 2:92066999 | 10128 | 2:92069312 | 10129 | 2:92085813 |
| 10130 | 2:92089545 | 10160 | 2:92756276 | 10181 | 2:93326093 | 10199 | 2:93469951 | 10200 | 2:93470548 |
| 10201 | 2:93471304 | 10202 | 2:93472560 | 10203 | 2:93473816 | 10218 | 2:93915068 | 10222 | 2:94006514 |
| 10223 | 2:94010942 | 10280 | 2:95335798 | 10338 | 2:96515244 | 10370 | 2:97455980 | 10377 | 2:97722574 |
| 10387 | 2:97882323 | 10401 | 2:98337087 | 10410 | 2:98432307 | 10413 | 2:98461459 | 10468 | 2:99951594 |
| 10492 | 2:100538824 | 10513 | 2:101147607 | 10555 | 2:102376439 | 10596 | 2:103531207 | 10650 | 2:104919346 |
| 10658 | 2:105027822 | 10663 | 2:105088054 | 10675 | 2:105412051 | 10829 | 2:108708703 | 10863 | 2:109439523 |
| 11027 | 2:113672892 | 11061 | 2:114526247 | 11202 | 2:118124674 | 11224 | 2:118587782 | 11225 | 2:118669657 |
| 11279 | 2:119878226 | 11336 | 2:121220519 | 11355 | 2:121820038 | 11356 | 2:121865885 | 11406 | 2:123297173 |
| 11455 | 2:124641406 | 11484 | 2:125479428 | 11617 | 2:127271059 | 11649 | 2:127896093 | 11743 | 2:130052119 |
| 11744 | 2:130058714 | 11745 | 2:130070890 | 11859 | 2:132478188 | 11860 | 2:132523448 | 11870 | 2:132734429 |
| 11917 | 2:133633752 | 11918 | 2:133637575 | 11939 | 2:133848234 | 11953 | 2:134015104 | 12115 | 3:525997 |
| 12118 | 3:556754 | 12143 | 3:950659 | 12145 | 3:954886 | 12153 | 3:1098608 | 12197 | 3:1960686 |
| 12261 | 3:3372085 | 12263 | 3:3434999 | 12280 | 3:3763587 | 12294 | 3:4101542 | 12315 | 3:4378889 |
| 12321 | 3:4437644 | 12343 | 3:4727841 | 12348 | 3:4795643 | 12369 | 3:5161089 | 12400 | 3:5776030 |
| 12410 | 3:6048242 | 12493 | 3:7480890 | 12527 | 3:7942262 | 12558 | 3:8433782 | 12561 | 3:8460244 |
| 12563 | 3:8520080 | 12564 | 3:8526638 | 12565 | 3:8529009 | 12566 | 3:8532372 | 12617 | 3:10020876 |
| 12620 | 3:10090524 | 12736 | 3:11477492 | 12754 | 3:11784186 | 12882 | 3:14796654 | 12934 | 3:15837312 |
| 13018 | 3:17753548 | 13048 | 3:18771436 | 13049 | 3:18775018 | 13050 | 3:18791656 | 13051 | 3:18795346 |
| 13056 | 3:18930543 | 13237 | 3:23451211 | 13314 | 3:25255575 | 13325 | 3:25405368 | 13343 | 3:25977712 |
| 13383 | 3:26543166 | 13431 | 3:27586116 | 13490 | 3:28659529 | 13578 | 3:30031426 | 13589 | 3:30407397 |
| 13632 | 3:31383515 | 13728 | 3:32928510 | 13731 | 3:32940407 | 13891 | 3:35285151 | 13909 | 3:35786483 |
| 13913 | 3:35908304 | 13922 | 3:36096832 | 13927 | 3:36253878 | 13990 | 3:37885414 | 14009 | 3:38624906 |
| 14010 | 3:38630748 | 14044 | 3:39845695 | 14058 | 3:40207827 | 14122 | 3:42050876 | 14139 | 3:42539907 |
| 14163 | 3:42995977 | 14232 | 3:44866434 | 14327 | 3:47649002 | 14398 | 3:49141481 | 14485 | 3:50111050 |
| 14521 | 3:51439192 | 14529 | 3:51625928 | 14618 | 3:53775526 | 14654 | 3:55087957 | 14667 | 3:55359809 |
| 14686 | 3:56006266 | 14688 | 3:56067950 | 14793 | 3:59053277 | 14797 | 3:59211837 | 14806 | 3:59443494 |
| 14818 | 3:59847747 | 14845 | 3:60575455 | 14847 | 3:60611151 | 14982 | 3:64188398 | 15009 | 3:65159985 |
| 15035 | 3:65721928 | 15088 | 3:66944634 | 15124 | 3:67769302 | 15169 | 3:68874408 | 15180 | 3:69234087 |
| 15187 | 3:69431230 | 15225 | 3:70347328 | 15264 | 3:72041156 | 15338 | 3:74664014 | 15421 | 3:77158063 |
| 15495 | 3:79128870 | 15545 | 3:80676731 | 15627 | 3:82625118 | 15652 | 3:83549724 | 15659 | 3:83658003 |
| 15683 | 3:84227201 | 15750 | 3:85215781 | 15768 | 3:85766170 | 15778 | 3:85960283 | 15779 | 3:85964163 |
| 15780 | 3:85967104 | 15781 | 3:85968892 | 15782 | 3:85969726 | 15798 | 3:86300913 | 15807 | 3:86441083 |
| 15908 | 3:88661891 | 15916 | 3:88782949 | 15917 | 3:88784034 | 15935 | 3:89220299 | 15977 | 3:89929116 |
| 16037 | 3:91288695 | 16170 | 3:94375264 | 16194 | 3:95152254 | 16204 | 3:95487322 | 16235 | 3:96416999 |
| 16276 | 3:97475924 | 16328 | 3:98728026 | 16330 | 3:98766843 | 16366 | 3:99717720 | 16400 | 3:100301019 |
| 16439 | 3:100971847 | 16445 | 3:101130049 | 16459 | 3:101339407 | 16485 | 3:101926220 | 16537 | 3:102883747 |
| 16572 | 3:103741473 | 16602 | 3:104484207 | 16605 | 3:104506752 | 16620 | 3:104710861 | 16639 | 3:105175424 |
| 16646 | 3:105209686 | 16684 | 3:105603312 | 16714 | 3:105965567 | 16715 | 3:105973059 | 16740 | 3:106492826 |
| 16774 | 3:107138615 | 16811 | 3:107842370 | 16874 | 3:109092493 | 16881 | 3:109399680 | 16887 | 3:109428951 |
| 16913 | 3:109808282 | 16959 | 3:110964139 | 17043 | 3:112687905 | 17134 | 3:114337662 | 17162 | 3:114723123 |
| 17166 | 3:114787475 | 17167 | 3:114787630 | 17168 | 3:114788049 | 17169 | 3:114788632 | 17170 | 3:114789131 |
| 17172 | 3:114791529 | 17173 | 3:114799492 | 17176 | 3:114803442 | 17177 | 3:114803451 | 17178 | 3:114803465 |
| 17179 | 3:114804161 | 17180 | 3:114804367 | 17195 | 3:115056511 | 17196 | 3:115071383 | 17206 | 3:115299586 |
| 17211 | 3:115381373 | 17407 | 3:118290616 | 17448 | 3:118879237 | 17676 | 4:4683962 | 17696 | 4:5003827 |
| 17732 | 4:5560351 | 17733 | 4:5568155 | 17760 | 4:6327312 | 17789 | 4:7010182 | 17804 | 4:7324916 |
| 17806 | 4:7326126 | 17855 | 4:8442378 | 17985 | 4:10842025 | 18045 | 4:12514592 | 18143 | 4:15186933 |
| 18155 | 4:15475039 | 18161 | 4:15588873 | 18162 | 4:15592728 | 18180 | 4:16210509 | 18257 | 4:18112247 |
| 18263 | 4:18273472 | 18273 | 4:18595000 | 18299 | 4:18985118 | 18400 | 4:20821405 | 18441 | 4:21738699 |
| 18444 | 4:21789346 | 18448 | 4:21942101 | 18464 | 4:22381610 | 18492 | 4:22930826 | 18520 | 4:23861032 |
| 18617 | 4:26517471 | 18748 | 4:30365149 | 18770 | 4:30805170 | 18813 | 4:31905587 | 18817 | 4:31972387 |
| 18879 | 4:33771936 | 18945 | 4:35882843 | 19025 | 4:38124757 | 19039 | 4:38555013 | 19086 | 4:40002914 |
| 19087 | 4:40007566 | 19088 | 4:40012097 | 19089 | 4:40018785 | 19090 | 4:40021130 | 19091 | 4:40041810 |
| 19092 | 4:40049842 | 19093 | 4:40054177 | 19094 | 4:40059057 | 19095 | 4:40060306 | 19115 | 4:40634211 |
| 19116 | 4:40640519 | 19117 | 4:40645360 | 19118 | 4:40649968 | 19119 | 4:40654713 | 19167 | 4:41814723 |
| 19172 | 4:42014141 | 19183 | 4:42356867 | 19190 | 4:42554917 | 19255 | 4:44244202 | 19291 | 4:45415922 |
| 19296 | 4:45536174 | 19308 | 4:45825175 | 19335 | 4:46718903 | 19342 | 4:46848225 | 19357 | 4:47216690 |
| 19358 | 4:47217662 | 19359 | 4:47221067 | 19360 | 4:47224473 | 19361 | 4:47228083 | 19400 | 4:48100380 |
| 19463 | 4:49408307 | 19575 | 4:53093575 | 19600 | 4:53722558 | 19607 | 4:53807295 | 19720 | 4:57434269 |
| 19763 | 4:58913134 | 19783 | 4:59391253 | 19787 | 4:59489126 | 19813 | 4:60378132 | 19818 | 4:60482892 |
| 19853 | 4:61414628 | 19854 | 4:61423853 | 19855 | 4:61432230 | 19856 | 4:61434936 | 19857 | 4:61439997 |
| 19881 | 4:62128232 | 19885 | 4:62171821 | 19916 | 4:63134475 | 19951 | 4:63940606 | 19960 | 4:64270176 |
| 19961 | 4:64271209 | 19964 | 4:64495002 | 19995 | 4:65572773 | 20043 | 4:66416290 | 20063 | 4:67092276 |
| 20082 | 4:67523624 | 20114 | 4:68300881 | 20190 | 4:70507936 | 20193 | 4:70569760 | 20246 | 4:72066682 |
| 20297 | 4:73725730 | 20302 | 4:73904776 | 20343 | 4:74843205 | 20361 | 4:75190744 | 20382 | 4:75792193 |
| 20390 | 4:75908626 | 20442 | 4:77149758 | 20462 | 4:77552330 | 20482 | 4:77954957 | 20489 | 4:78153455 |
| 20519 | 4:79093225 | 20523 | 4:79236024 | 20527 | 4:79350296 | 20537 | 4:79637506 | 20607 | 4:81119261 |
| 20687 | 4:83090213 | 20854 | 4:87615934 | 20860 | 4:87832774 | 20862 | 4:87887042 | 20866 | 4:87931932 |
| 20867 | 4:87937433 | 20918 | 4:89596848 | 20927 | 4:89756186 | 20933 | 4:89854090 | 20952 | 4:90405270 |
| 20965 | 4:90752463 | 20983 | 4:91186390 | 21013 | 4:91662043 | 21020 | 4:91835951 | 21026 | 4:91985689 |
| 21064 | 4:92683225 | 21143 | 4:94586866 | 21162 | 4:95121580 | 21208 | 4:95976133 | 21209 | 4:95978012 |
| 21210 | 4:95984102 | 21211 | 4:95986472 | 21212 | 4:95988223 | 21213 | 4:95991905 | 21214 | 4:95994577 |
| 21215 | 4:95996626 | 21216 | 4:95999064 | 21217 | 4:95999599 | 21218 | 4:95999657 | 21220 | 4:96006244 |
| 21223 | 4:96012342 | 21224 | 4:96014135 | 21225 | 4:96014452 | 21226 | 4:96014495 | 21229 | 4:96043384 |
| 21230 | 4:96043881 | 21258 | 4:96806394 | 21259 | 4:96808296 | 21260 | 4:96808305 | 21261 | 4:96808793 |
| 21263 | 4:96810164 | 21264 | 4:96810688 | 21265 | 4:96810739 | 21266 | 4:96811491 | 21267 | 4:96811492 |
| 21268 | 4:96811663 | 21269 | 4:96819713 | 21270 | 4:96819807 | 21271 | 4:96819821 | 21272 | 4:96820983 |
| 21273 | 4:96821494 | 21274 | 4:96822544 | 21275 | 4:96822709 | 21276 | 4:96822722 | 21277 | 4:96823395 |
| 21278 | 4:96823570 | 21280 | 4:96826910 | 21281 | 4:96827344 | 21282 | 4:96828046 | 21283 | 4:96828753 |
| 21284 | 4:96829524 | 21286 | 4:96842438 | 21287 | 4:96842474 | 21288 | 4:96846828 | 21289 | 4:96847175 |
| 21290 | 4:96847270 | 21291 | 4:96847298 | 21292 | 4:96847522 | 21293 | 4:96847685 | 21294 | 4:96848163 |
| 21295 | 4:96852063 | 21296 | 4:96852112 | 21297 | 4:96852128 | 21299 | 4:96853114 | 21300 | 4:96853149 |
| 21301 | 4:96854041 | 21302 | 4:96854052 | 21303 | 4:96854217 | 21304 | 4:96854309 | 21305 | 4:96854674 |
| 21306 | 4:96854753 | 21307 | 4:96854840 | 21308 | 4:96854846 | 21309 | 4:96855073 | 21310 | 4:96855142 |
| 21311 | 4:96855211 | 21312 | 4:96855234 | 21313 | 4:96855382 | 21314 | 4:96858356 | 21315 | 4:96858411 |
| 21316 | 4:96858620 | 21317 | 4:96859305 | 21318 | 4:96859677 | 21319 | 4:96859857 | 21320 | 4:96860289 |
| 21321 | 4:96860821 | 21322 | 4:96861792 | 21323 | 4:96862653 | 21324 | 4:96868903 | 21325 | 4:96870132 |
| 21326 | 4:96870621 | 21327 | 4:96871036 | 21328 | 4:96871329 | 21329 | 4:96872714 | 21330 | 4:96872734 |
| 21331 | 4:96878187 | 21332 | 4:96878242 | 21333 | 4:96878278 | 21334 | 4:96880890 | 21335 | 4:96880962 |
| 21336 | 4:96881026 | 21337 | 4:96881161 | 21338 | 4:96881966 | 21339 | 4:96882244 | 21340 | 4:96885170 |
| 21341 | 4:96886853 | 21342 | 4:96887160 | 21343 | 4:96887442 | 21344 | 4:96887486 | 21345 | 4:96887810 |
| 21346 | 4:96888595 | 21347 | 4:96889148 | 21348 | 4:96890359 | 21349 | 4:96890450 | 21350 | 4:96890701 |
| 21351 | 4:96891069 | 21352 | 4:96891335 | 21353 | 4:96891542 | 21354 | 4:96891552 | 21355 | 4:96891608 |
| 21356 | 4:96895620 | 21357 | 4:96897050 | 21358 | 4:96897145 | 21367 | 4:97039076 | 21370 | 4:97088268 |
| 21372 | 4:97137855 | 21373 | 4:97147752 | 21374 | 4:97148005 | 21375 | 4:97148878 | 21376 | 4:97150124 |
| 21377 | 4:97150300 | 21378 | 4:97151421 | 21379 | 4:97151944 | 21380 | 4:97152001 | 21381 | 4:97152026 |
| 21382 | 4:97152080 | 21383 | 4:97152151 | 21384 | 4:97152177 | 21385 | 4:97152300 | 21386 | 4:97152327 |
| 21387 | 4:97152343 | 21388 | 4:97152464 | 21389 | 4:97152562 | 21390 | 4:97152880 | 21391 | 4:97153058 |
| 21392 | 4:97160379 | 21393 | 4:97160479 | 21394 | 4:97160507 | 21395 | 4:97160725 | 21396 | 4:97160764 |
| 21397 | 4:97160849 | 21398 | 4:97160967 | 21399 | 4:97161387 | 21400 | 4:97161554 | 21401 | 4:97161881 |
| 21402 | 4:97161958 | 21404 | 4:97164436 | 21405 | 4:97165222 | 21406 | 4:97165446 | 21407 | 4:97165671 |
| 21408 | 4:97165867 | 21409 | 4:97167273 | 21410 | 4:97170350 | 21411 | 4:97170722 | 21412 | 4:97170758 |
| 21413 | 4:97170786 | 21415 | 4:97171793 | 21417 | 4:97173637 | 21418 | 4:97173882 | 21420 | 4:97243175 |
| 21429 | 4:97478233 | 21449 | 4:97997496 | 21460 | 4:98301073 | 21488 | 4:99227319 | 21496 | 4:99551602 |
| 21525 | 4:100096858 | 21571 | 4:101147322 | 21621 | 4:102291227 | 21665 | 4:103373158 | 21709 | 4:104494825 |
| 21712 | 4:104541226 | 21740 | 4:105243840 | 21826 | 4:106811124 | 21828 | 4:106840758 | 21903 | 4:108136633 |
| 21927 | 4:108959151 | 21976 | 4:110025220 | 22029 | 4:111137956 | 22031 | 4:111174901 | 22032 | 4:111185441 |
| 22052 | 4:111594642 | 22057 | 4:111656507 | 22081 | 4:112008507 | 22182 | 4:113533731 | 22254 | 4:114468144 |
| 22334 | 4:115504063 | 22339 | 4:115674188 | 22348 | 4:115903138 | 22484 | 4:118639815 | 22639 | 5:1916328 |
| 22664 | 5:2361885 | 22679 | 5:2678940 | 22680 | 5:2710438 | 22682 | 5:2775410 | 22683 | 5:2789169 |
| 22692 | 5:3084344 | 22721 | 5:4106293 | 22747 | 5:4642231 | 22801 | 5:5757045 | 22812 | 5:5962601 |
| 22864 | 5:7150201 | 22875 | 5:7320087 | 22901 | 5:8063807 | 22907 | 5:8295061 | 22914 | 5:8547280 |
| 23001 | 5:10417113 | 23005 | 5:10483003 | 23116 | 5:12929697 | 23147 | 5:13814938 | 23154 | 5:14118702 |
| 23200 | 5:15181434 | 23209 | 5:15382347 | 23335 | 5:18879061 | 23345 | 5:19180052 | 23365 | 5:19703644 |
| 23420 | 5:21141526 | 23424 | 5:21247771 | 23441 | 5:21652414 | 23488 | 5:22524662 | 23553 | 5:23856635 |
| 23577 | 5:24327813 | 23580 | 5:24363604 | 23609 | 5:25025028 | 23638 | 5:25910383 | 23667 | 5:26553269 |
| 23765 | 5:28658495 | 23768 | 5:28689345 | 23795 | 5:29481439 | 23827 | 5:30068971 | 23860 | 5:30880544 |
| 23885 | 5:31649328 | 23903 | 5:32253508 | 23967 | 5:33731249 | 23969 | 5:33743644 | 23990 | 5:34527357 |
| 24023 | 5:35215343 | 24041 | 5:35917350 | 24064 | 5:36532219 | 24164 | 5:39219011 | 24213 | 5:40323694 |
| 24252 | 5:41581614 | 24255 | 5:41737754 | 24277 | 5:42488185 | 24295 | 5:42883310 | 24309 | 5:43239107 |
| 24321 | 5:43556568 | 24332 | 5:43826757 | 24373 | 5:44749275 | 24394 | 5:45422250 | 24412 | 5:45892148 |
| 24469 | 5:47071100 | 24474 | 5:47204283 | 24476 | 5:47210190 | 24491 | 5:47544371 | 24515 | 5:48255910 |
| 24533 | 5:48697507 | 24700 | 5:53392371 | 24725 | 5:54050541 | 24767 | 5:54925075 | 24868 | 5:57315947 |
| 24869 | 5:57316513 | 24870 | 5:57318993 | 24931 | 5:59393649 | 25012 | 5:61592328 | 25049 | 5:62915639 |
| 25065 | 5:63217741 | 25070 | 5:63272705 | 25094 | 5:63826831 | 25189 | 5:66342506 | 25206 | 5:66729461 |
| 25248 | 5:67796979 | 25274 | 5:68252711 | 25308 | 5:69044504 | 25336 | 5:69658212 | 25386 | 5:70977990 |
| 25396 | 5:71157250 | 25405 | 5:71284786 | 25414 | 5:71441252 | 25415 | 5:71451984 | 25452 | 5:72240212 |
| 25455 | 5:72267018 | 25501 | 5:73577352 | 25505 | 5:73676579 | 25512 | 5:73764801 | 25516 | 5:73813825 |
| 25528 | 5:74101395 | 25540 | 5:74482047 | 25592 | 5:75448318 | 25633 | 5:76668839 | 25639 | 5:76766433 |
| 25644 | 5:76890153 | 25683 | 5:77996585 | 25684 | 5:78020028 | 25716 | 5:78821705 | 25736 | 5:79345129 |
| 25744 | 5:79644695 | 25768 | 5:80587434 | 25796 | 5:81407773 | 25800 | 5:81437991 | 25801 | 5:81441963 |
| 25851 | 5:82570119 | 25855 | 5:82593018 | 25856 | 5:82593228 | 25857 | 5:82593982 | 25858 | 5:82594019 |
| 25859 | 5:82594028 | 25860 | 5:82594375 | 25861 | 5:82594380 | 25862 | 5:82595065 | 25863 | 5:82595148 |
| 25864 | 5:82595334 | 25865 | 5:82627761 | 25905 | 5:83646235 | 26064 | 5:84298547 | 26072 | 5:84433271 |
| 26074 | 5:84527341 | 26089 | 5:84948836 | 26145 | 5:86376332 | 26148 | 5:86455776 | 26196 | 5:87435829 |
| 26293 | 5:88856288 | 26361 | 5:90566853 | 26403 | 5:91501158 | 26409 | 5:91665913 | 26415 | 5:91761326 |
| 26432 | 5:92332808 | 26496 | 5:93178439 | 26526 | 5:93790346 | 26533 | 5:93961735 | 26537 | 5:94088171 |
| 26579 | 5:95091626 | 26625 | 5:96054240 | 26685 | 5:97071869 | 26781 | 5:98982241 | 26878 | 5:100843102 |
| 26891 | 5:101049907 | 26948 | 5:102626302 | 27117 | 5:105882827 | 27183 | 5:106801714 | 27187 | 5:106877986 |
| 27206 | 5:107060379 | 27244 | 5:107745197 | 27305 | 5:108794780 | 27306 | 5:108797633 | 27341 | 5:109446738 |
| 27366 | 5:109926959 | 27428 | 5:111036587 | 27579 | 5:114683035 | 27584 | 5:114814526 | 27611 | 5:115497329 |
| 27613 | 5:115504227 | 27623 | 5:115614570 | 27664 | 5:116449909 | 27776 | 5:117284799 | 27782 | 5:117411822 |
| 27811 | 5:117753977 | 27824 | 5:117977735 | 27839 | 5:118288125 | 27856 | 5:118666844 | 27917 | 5:119642118 |
| 27951 | 6:579203 | 28082 | 6:3248784 | 28098 | 6:3406400 | 28122 | 6:3655995 | 28164 | 6:4606383 |
| 28201 | 6:5673122 | 28210 | 6:5813338 | 28269 | 6:6797112 | 28271 | 6:6808370 | 28384 | 6:9740976 |
| 28388 | 6:9896356 | 28418 | 6:10695206 | 28487 | 6:12244147 | 28494 | 6:12408823 | 28501 | 6:12515175 |
| 28516 | 6:12899581 | 28534 | 6:13157939 | 28535 | 6:13158398 | 28536 | 6:13158702 | 28570 | 6:14149976 |
| 28645 | 6:15766628 | 28649 | 6:15875705 | 28664 | 6:16165237 | 28720 | 6:17350197 | 28729 | 6:17546682 |
| 28775 | 6:18247972 | 28798 | 6:18858161 | 28832 | 6:19517669 | 28855 | 6:20067345 | 28903 | 6:21187469 |
| 28951 | 6:22198236 | 28974 | 6:22627641 | 28985 | 6:22838005 | 28994 | 6:23185445 | 29012 | 6:23551846 |
| 29033 | 6:24079559 | 29091 | 6:25420968 | 29092 | 6:25430886 | 29113 | 6:25987389 | 29124 | 6:26122133 |
| 29169 | 6:27357262 | 29187 | 6:27922767 | 29228 | 6:29044156 | 29256 | 6:29526067 | 29268 | 6:29755371 |
| 29360 | 6:30504972 | 29389 | 6:31372994 | 29391 | 6:31402252 | 29427 | 6:32495711 | 29446 | 6:33106068 |
| 29505 | 6:34316541 | 29908 | 6:34880169 | 29988 | 6:35895370 | 29990 | 6:35897546 | 29991 | 6:35897939 |
| 29992 | 6:35906185 | 29993 | 6:35907046 | 29995 | 6:35909397 | 29998 | 6:35925489 | 30025 | 6:36097722 |
| 30135 | 6:36427342 | 30137 | 6:36430013 | 30140 | 6:36432356 | 30143 | 6:36441375 | 30257 | 6:36563183 |
| 30260 | 6:36568261 | 30264 | 6:36572899 | 30333 | 6:36813124 | 30455 | 6:37026863 | 30456 | 6:37027429 |
| 30457 | 6:37027539 | 30458 | 6:37028623 | 30460 | 6:37030480 | 30461 | 6:37032906 | 30462 | 6:37032995 |
| 30467 | 6:37035186 | 30469 | 6:37038017 | 30471 | 6:37056413 | 30475 | 6:37075910 | 30566 | 6:37095299 |
| 30616 | 6:37107589 | 30626 | 6:37117771 | 30629 | 6:37121529 | 30630 | 6:37123023 | 30632 | 6:37130785 |
| 30633 | 6:37134418 | 30636 | 6:37136794 | 30643 | 6:37142174 | 30649 | 6:37149945 | 30685 | 6:37161408 |
| 30736 | 6:37173048 | 30788 | 6:37184577 | 30791 | 6:37188623 | 30792 | 6:37189736 | 30798 | 6:37206928 |
| 30799 | 6:37210690 | 30802 | 6:37214389 | 30803 | 6:37214736 | 30810 | 6:37219609 | 30813 | 6:37221776 |
| 30818 | 6:37227250 | 30820 | 6:37231623 | 30829 | 6:37235373 | 30830 | 6:37235374 | 30831 | 6:37236226 |
| 30844 | 6:37244478 | 30849 | 6:37247874 | 30851 | 6:37250850 | 30852 | 6:37251132 | 30854 | 6:37252978 |
| 30856 | 6:37256488 | 30857 | 6:37260221 | 30858 | 6:37262825 | 30859 | 6:37262995 | 30864 | 6:37269061 |
| 30865 | 6:37270215 | 30866 | 6:37270930 | 30867 | 6:37271635 | 30870 | 6:37281308 | 30871 | 6:37281335 |
| 30872 | 6:37282356 | 30873 | 6:37284874 | 30879 | 6:37286627 | 30880 | 6:37286722 | 30881 | 6:37287306 |
| 30882 | 6:37287423 | 30883 | 6:37287873 | 30884 | 6:37288219 | 30885 | 6:37288483 | 30886 | 6:37288600 |
| 30887 | 6:37288948 | 30888 | 6:37289071 | 30889 | 6:37289572 | 30890 | 6:37289742 | 30891 | 6:37289905 |
| 30892 | 6:37289990 | 30893 | 6:37290180 | 30894 | 6:37290241 | 30895 | 6:37290415 | 30896 | 6:37291360 |
| 30897 | 6:37291500 | 30898 | 6:37292117 | 30899 | 6:37292269 | 30900 | 6:37292270 | 30901 | 6:37292298 |
| 30902 | 6:37292752 | 30903 | 6:37293476 | 30905 | 6:37294505 | 30913 | 6:37297426 | 30917 | 6:37298725 |
| 30927 | 6:37300810 | 30932 | 6:37301959 | 30936 | 6:37302499 | 30938 | 6:37303255 | 30946 | 6:37304513 |
| 30952 | 6:37305466 | 30956 | 6:37306944 | 30959 | 6:37309266 | 30960 | 6:37310413 | 30972 | 6:37313503 |
| 30977 | 6:37314183 | 30993 | 6:37317695 | 31003 | 6:37321049 | 31004 | 6:37321441 | 31010 | 6:37324718 |
| 31018 | 6:37328528 | 31026 | 6:37329878 | 31030 | 6:37330306 | 31032 | 6:37330477 | 31033 | 6:37330577 |
| 31036 | 6:37331712 | 31046 | 6:37335926 | 31052 | 6:37337744 | 31056 | 6:37340937 | 31058 | 6:37342568 |
| 31066 | 6:37345508 | 31070 | 6:37347130 | 31071 | 6:37349144 | 31075 | 6:37351767 | 31076 | 6:37352145 |
| 31086 | 6:37359765 | 31088 | 6:37360685 | 31089 | 6:37361449 | 31090 | 6:37361578 | 31091 | 6:37362262 |
| 31099 | 6:37369338 | 31100 | 6:37369380 | 31102 | 6:37372334 | 31108 | 6:37376289 | 31111 | 6:37378475 |
| 31113 | 6:37379506 | 31114 | 6:37379507 | 31118 | 6:37380161 | 31120 | 6:37381106 | 31122 | 6:37381782 |
| 31123 | 6:37382003 | 31124 | 6:37382062 | 31127 | 6:37383526 | 31129 | 6:37385973 | 31133 | 6:37391915 |
| 31138 | 6:37393949 | 31139 | 6:37394877 | 31140 | 6:37395313 | 31143 | 6:37396789 | 31144 | 6:37398199 |
| 31145 | 6:37399560 | 31147 | 6:37400741 | 31151 | 6:37403131 | 31152 | 6:37403224 | 31153 | 6:37406239 |
| 31154 | 6:37406485 | 31156 | 6:37407653 | 31157 | 6:37412062 | 31162 | 6:37415367 | 31163 | 6:37416259 |
| 31166 | 6:37417501 | 31167 | 6:37418164 | 31169 | 6:37418447 | 31170 | 6:37418449 | 31172 | 6:37420957 |
| 31174 | 6:37424248 | 31176 | 6:37429071 | 31178 | 6:37429776 | 31179 | 6:37430447 | 31180 | 6:37431101 |
| 31181 | 6:37432458 | 31186 | 6:37435861 | 31188 | 6:37436347 | 31191 | 6:37437538 | 31192 | 6:37438222 |
| 31197 | 6:37438993 | 31199 | 6:37440571 | 31204 | 6:37444900 | 31208 | 6:37449890 | 31212 | 6:37457249 |
| 31216 | 6:37465669 | 31217 | 6:37466194 | 31218 | 6:37473453 | 31219 | 6:37477629 | 31221 | 6:37480277 |
| 31222 | 6:37482054 | 31225 | 6:37491305 | 31229 | 6:37503406 | 31230 | 6:37505093 | 31231 | 6:37508184 |
| 31235 | 6:37513645 | 31239 | 6:37522580 | 31240 | 6:37526812 | 31243 | 6:37534297 | 31246 | 6:37553481 |
| 31248 | 6:37560625 | 31249 | 6:37562383 | 31251 | 6:37566268 | 31252 | 6:37566317 | 31254 | 6:37573307 |
| 31255 | 6:37573615 | 31256 | 6:37578832 | 31262 | 6:37587980 | 31263 | 6:37588492 | 31264 | 6:37591326 |
| 31265 | 6:37591664 | 31268 | 6:37598284 | 31276 | 6:37608504 | 31278 | 6:37614179 | 31280 | 6:37624003 |
| 31281 | 6:37625923 | 31282 | 6:37627776 | 31284 | 6:37636883 | 31285 | 6:37636900 | 31286 | 6:37637484 |
| 31287 | 6:37639651 | 31288 | 6:37639763 | 31289 | 6:37640954 | 31290 | 6:37641448 | 31291 | 6:37641798 |
| 31292 | 6:37642775 | 31293 | 6:37644691 | 31294 | 6:37644915 | 31295 | 6:37645671 | 31296 | 6:37646000 |
| 31297 | 6:37646499 | 31301 | 6:37658848 | 31349 | 6:37772675 | 31353 | 6:37774778 | 31355 | 6:37775624 |
| 31357 | 6:37776401 | 31359 | 6:37776454 | 31360 | 6:37776464 | 31361 | 6:37777029 | 31363 | 6:37777185 |
| 31404 | 6:37817575 | 31438 | 6:37912116 | 31445 | 6:37914472 | 31454 | 6:37917366 | 31455 | 6:37917421 |
| 31465 | 6:37919853 | 31475 | 6:37926073 | 31476 | 6:37926600 | 31477 | 6:37926960 | 31478 | 6:37928156 |
| 31484 | 6:37929786 | 31486 | 6:37930252 | 31487 | 6:37930293 | 31488 | 6:37930672 | 31490 | 6:37930868 |
| 31491 | 6:37930889 | 31492 | 6:37931089 | 31493 | 6:37931216 | 31494 | 6:37931298 | 31495 | 6:37931621 |
| 31496 | 6:37931688 | 31499 | 6:37932170 | 31501 | 6:37932241 | 31503 | 6:37932377 | 31504 | 6:37932412 |
| 31506 | 6:37932563 | 31507 | 6:37932682 | 31508 | 6:37932772 | 31509 | 6:37932778 | 31510 | 6:37932960 |
| 31512 | 6:37933352 | 31514 | 6:37934068 | 31518 | 6:37935108 | 31519 | 6:37935168 | 31520 | 6:37935527 |
| 31521 | 6:37935577 | 31522 | 6:37935748 | 31524 | 6:37936087 | 31525 | 6:37936692 | 31526 | 6:37938294 |
| 31527 | 6:37938374 | 31528 | 6:37938826 | 31531 | 6:37942290 | 31532 | 6:37942944 | 31533 | 6:37943635 |
| 31534 | 6:37943860 | 31535 | 6:37943970 | 31536 | 6:37944478 | 31537 | 6:37944482 | 31538 | 6:37944496 |
| 31539 | 6:37944573 | 31541 | 6:37944681 | 31542 | 6:37944704 | 31544 | 6:37945482 | 31545 | 6:37945820 |
| 31546 | 6:37945849 | 31558 | 6:37959360 | 31561 | 6:37961553 | 31563 | 6:37962887 | 31565 | 6:37963525 |
| 31568 | 6:37966382 | 31585 | 6:37974401 | 31586 | 6:37974407 | 31819 | 6:38395086 | 31822 | 6:38409517 |
| 31826 | 6:38491475 | 31831 | 6:38518347 | 31836 | 6:38540452 | 31837 | 6:38541525 | 31839 | 6:38542048 |
| 31842 | 6:38543400 | 31843 | 6:38543545 | 31844 | 6:38543990 | 31845 | 6:38545112 | 31846 | 6:38545113 |
| 31847 | 6:38546290 | 31848 | 6:38546646 | 31849 | 6:38548060 | 31850 | 6:38548198 | 31851 | 6:38548496 |
| 31852 | 6:38548525 | 31853 | 6:38548659 | 31854 | 6:38549044 | 31855 | 6:38549099 | 31856 | 6:38549206 |
| 31857 | 6:38549477 | 31859 | 6:38550162 | 31860 | 6:38550667 | 31861 | 6:38552085 | 31862 | 6:38552158 |
| 31863 | 6:38552167 | 31864 | 6:38552459 | 31865 | 6:38552550 | 31866 | 6:38552644 | 31867 | 6:38552821 |
| 31868 | 6:38553028 | 31869 | 6:38553181 | 31870 | 6:38553447 | 31871 | 6:38553456 | 31872 | 6:38553565 |
| 31873 | 6:38553842 | 31874 | 6:38553946 | 31875 | 6:38554100 | 31876 | 6:38554163 | 31877 | 6:38554439 |
| 31878 | 6:38554455 | 31879 | 6:38554714 | 31880 | 6:38555555 | 31881 | 6:38556220 | 31882 | 6:38556748 |
| 31883 | 6:38556956 | 31884 | 6:38556978 | 31885 | 6:38557241 | 31886 | 6:38557498 | 31887 | 6:38557633 |
| 31888 | 6:38557694 | 31889 | 6:38557830 | 31890 | 6:38557833 | 31891 | 6:38557888 | 31892 | 6:38557940 |
| 31894 | 6:38558946 | 31895 | 6:38562229 | 31897 | 6:38564166 | 31898 | 6:38565084 | 31902 | 6:38583433 |
| 31904 | 6:38584686 | 31908 | 6:38600235 | 31965 | 6:39250952 | 31969 | 6:39301737 | 31989 | 6:39663383 |
| 31992 | 6:39721697 | 31993 | 6:39727899 | 31994 | 6:39730068 | 31995 | 6:39733610 | 31996 | 6:39737879 |
| 32029 | 6:40129309 | 32034 | 6:40237863 | 32062 | 6:40696320 | 32086 | 6:40735514 | 32119 | 6:40952449 |
| 32207 | 6:42123221 | 32266 | 6:43009159 | 32267 | 6:43017852 | 32294 | 6:43462079 | 32329 | 6:43973189 |
| 32339 | 6:44149286 | 32547 | 6:46220665 | 32588 | 6:47190072 | 32619 | 6:47907662 | 32631 | 6:48281817 |
| 32657 | 6:49188655 | 32670 | 6:49492652 | 32675 | 6:49628351 | 32678 | 6:49692019 | 32757 | 6:51655726 |
| 32824 | 6:52798083 | 32827 | 6:52912323 | 32854 | 6:53474037 | 32911 | 6:55287740 | 32947 | 6:55553127 |
| 32949 | 6:55563964 | 32951 | 6:55573386 | 32968 | 6:56049754 | 32972 | 6:56230949 | 33026 | 6:57612544 |
| 33034 | 6:57940998 | 33036 | 6:57996603 | 33038 | 6:58046666 | 33086 | 6:59279953 | 33101 | 6:59756158 |
| 33144 | 6:60551717 | 33151 | 6:60729399 | 33174 | 6:61339528 | 33199 | 6:61996360 | 33215 | 6:62563366 |
| 33218 | 6:62627183 | 33317 | 6:65798913 | 33363 | 6:67312644 | 33364 | 6:67348632 | 33404 | 6:68600503 |
| 33421 | 6:69030371 | 33437 | 6:69395469 | 33438 | 6:69429025 | 33455 | 6:70069795 | 33480 | 6:70868126 |
| 33492 | 6:71178118 | 33506 | 6:71614584 | 33509 | 6:71658863 | 33575 | 6:73737226 | 33611 | 6:75157043 |
| 33635 | 6:75976163 | 33715 | 6:78625022 | 33873 | 6:83484610 | 33909 | 6:84465355 | 33927 | 6:84898498 |
| 34137 | 6:89248038 | 34177 | 6:90260850 | 34180 | 6:90360832 | 34292 | 6:92070503 | 34356 | 6:93561855 |
| 34416 | 6:94977849 | 34431 | 6:95495492 | 34452 | 6:96090013 | 34453 | 6:96097237 | 34476 | 6:96479912 |
| 34478 | 6:96544331 | 34486 | 6:96686083 | 34491 | 6:96916348 | 34531 | 6:97462721 | 34545 | 6:97701064 |
| 34600 | 6:98712630 | 34635 | 6:99753620 | 34663 | 6:100291469 | 34716 | 6:101484421 | 34717 | 6:101486616 |
| 34720 | 6:101524578 | 34735 | 6:101873336 | 34761 | 6:102268756 | 34817 | 6:103176226 | 34828 | 6:103236460 |
| 34912 | 6:105003956 | 35080 | 6:109427077 | 35101 | 6:109822389 | 35163 | 6:111118988 | 35165 | 6:111196630 |
| 35175 | 6:111382228 | 35229 | 6:112604266 | 35271 | 6:113397576 | 35275 | 6:113470683 | 35406 | 6:115996386 |
| 35448 | 6:116540874 | 35480 | 7:310937 | 35482 | 7:325799 | 35558 | 7:1927986 | 35561 | 7:1995591 |
| 35582 | 7:2406915 | 35845 | 7:6222553 | 35873 | 7:6554748 | 35887 | 7:6745523 | 35900 | 7:6956627 |
| 35928 | 7:7534130 | 35936 | 7:7674523 | 35947 | 7:8035405 | 35989 | 7:10310655 | 35990 | 7:10395846 |
| 36049 | 7:11976493 | 36068 | 7:12333943 | 36069 | 7:12335290 | 36140 | 7:13300617 | 36153 | 7:13562003 |
| 36173 | 7:13942658 | 36277 | 7:15601726 | 36310 | 7:16447387 | 36311 | 7:16487854 | 36362 | 7:17629628 |
| 36364 | 7:17639856 | 36392 | 7:18356451 | 36423 | 7:19107522 | 36440 | 7:19307460 | 36503 | 7:20153088 |
| 36525 | 7:20401996 | 36546 | 7:20912796 | 36597 | 7:22016441 | 36603 | 7:22057135 | 36620 | 7:22504243 |
| 36646 | 7:23221425 | 36663 | 7:23603215 | 36683 | 7:24060066 | 36685 | 7:24072015 | 36705 | 7:24634875 |
| 36768 | 7:26043336 | 36828 | 7:27103526 | 36858 | 7:27828159 | 36879 | 7:28407768 | 36890 | 7:28661620 |
| 36941 | 7:29715806 | 37014 | 7:31333882 | 37042 | 7:31763022 | 37047 | 7:31806849 | 37062 | 7:32255462 |
| 37091 | 7:33083761 | 37108 | 7:33452281 | 37175 | 7:35015402 | 37188 | 7:35370637 | 37211 | 7:35997017 |
| 37230 | 7:36507816 | 37294 | 7:37977197 | 37369 | 7:39696107 | 37371 | 7:39720514 | 37528 | 7:43355224 |
| 37544 | 7:43621545 | 37609 | 7:45530009 | 37635 | 7:46401887 | 37725 | 7:48628543 | 37738 | 7:48808046 |
| 37745 | 7:48945061 | 37752 | 7:49169638 | 37886 | 7:52686851 | 37912 | 7:53315105 | 37957 | 7:54541065 |
| 37960 | 7:54622430 | 37975 | 7:55088638 | 38007 | 7:55900961 | 38015 | 7:56078682 | 38033 | 7:56535413 |
| 38035 | 7:56546344 | 38057 | 7:57273769 | 38076 | 7:57910079 | 38077 | 7:57916334 | 38081 | 7:57993375 |
| 38083 | 7:58043785 | 38153 | 7:59374018 | 38159 | 7:59461244 | 38188 | 7:60116908 | 38196 | 7:60319670 |
| 38224 | 7:61001713 | 38269 | 7:61575267 | 38286 | 7:62059589 | 38299 | 7:62381127 | 38312 | 7:62665355 |
| 38313 | 7:62666818 | 38441 | 7:65409794 | 38449 | 7:65594792 | 38520 | 7:66847524 | 38592 | 7:68772935 |
| 38594 | 7:68885530 | 38657 | 7:70110273 | 38683 | 7:71032462 | 38718 | 7:71794897 | 38728 | 7:71996424 |
| 38742 | 7:72314529 | 38808 | 7:74314108 | 38814 | 7:74538065 | 38843 | 7:75119443 | 38849 | 7:75246858 |
| 38850 | 7:75278782 | 38865 | 7:75680840 | 38908 | 7:76699078 | 38924 | 7:77064173 | 38949 | 7:77643339 |
| 38981 | 7:78247892 | 38986 | 7:78358118 | 39067 | 7:79874457 | 39117 | 7:80683144 | 39127 | 7:80972557 |
| 39147 | 7:81218151 | 39197 | 7:82527838 | 39256 | 7:83861078 | 39276 | 7:84283959 | 39317 | 7:85606256 |
| 39369 | 7:87039982 | 39386 | 7:87585340 | 39438 | 7:89078282 | 39458 | 7:89409840 | 39473 | 7:89883975 |
| 39495 | 7:90326242 | 39589 | 7:91305083 | 39633 | 7:92190820 | 39704 | 7:94482521 | 39741 | 7:95245462 |
| 39764 | 7:95873877 | 39776 | 7:96101255 | 39780 | 7:96126223 | 39967 | 7:96962124 | 39977 | 7:97249811 |
| 39984 | 7:97508045 | 39987 | 7:97537955 | 40007 | 7:98034786 | 40009 | 7:98127942 | 40167 | 7:102360026 |
| 40187 | 7:102838629 | 40190 | 7:102879057 | 40252 | 7:104220122 | 40270 | 7:104812484 | 40345 | 7:106558358 |
| 40381 | 7:107255212 | 40401 | 7:108080872 | 40406 | 7:108274048 | 40425 | 7:108662049 | 40435 | 7:108882531 |
| 40540 | 8:1185446 | 40561 | 8:1731044 | 40562 | 8:1735452 | 40568 | 8:1797826 | 40584 | 8:2112938 |
| 40597 | 8:2381761 | 40609 | 8:2664904 | 40720 | 8:5502050 | 40787 | 8:7227540 | 40820 | 8:7866886 |
| 40852 | 8:8701133 | 40988 | 8:11839675 | 41027 | 8:12939122 | 41083 | 8:14812117 | 41084 | 8:14836734 |
| 41129 | 8:15805194 | 41130 | 8:15826955 | 41140 | 8:16188640 | 41161 | 8:16768290 | 41295 | 8:20088784 |
| 41367 | 8:22206603 | 41372 | 8:22296675 | 41379 | 8:22531070 | 41384 | 8:22832882 | 41391 | 8:23108057 |
| 41574 | 8:25836147 | 41585 | 8:26072917 | 41683 | 8:28986201 | 41684 | 8:29004667 | 41685 | 8:29005955 |
| 41710 | 8:29655591 | 41727 | 8:29969758 | 41806 | 8:32023600 | 41820 | 8:32402575 | 41826 | 8:32658867 |
| 41872 | 8:34205302 | 41887 | 8:34586231 | 41924 | 8:35462325 | 41970 | 8:36379322 | 41971 | 8:36399226 |
| 42048 | 8:38716745 | 42085 | 8:39915480 | 42086 | 8:39942972 | 42119 | 8:40483328 | 42137 | 8:40876799 |
| 42195 | 8:42367753 | 42266 | 8:44367476 | 42313 | 8:45640500 | 42355 | 8:46379679 | 42373 | 8:46931988 |
| 42389 | 8:47348455 | 42415 | 8:47938185 | 42433 | 8:48578484 | 42492 | 8:50146172 | 42546 | 8:51588079 |
| 42631 | 8:53806641 | 42639 | 8:54114529 | 42646 | 8:54256907 | 42654 | 8:54525130 | 42656 | 8:54533690 |
| 42657 | 8:54534313 | 42658 | 8:54536088 | 42659 | 8:54544973 | 42660 | 8:54547153 | 42854 | 8:59769666 |
| 42892 | 8:60472436 | 42903 | 8:60785213 | 43085 | 8:65667278 | 43089 | 8:65893239 | 43110 | 8:66320758 |
| 43186 | 8:67933084 | 43215 | 8:68749952 | 43230 | 8:69106626 | 43232 | 8:69110799 | 43244 | 8:69435453 |
| 43345 | 8:72058951 | 43397 | 8:73337772 | 43434 | 8:74223661 | 43566 | 8:77624055 | 43579 | 8:78130709 |
| 43593 | 8:78494713 | 43655 | 8:80608965 | 43668 | 8:80925878 | 43679 | 8:81135408 | 43745 | 8:82415599 |
| 43764 | 8:82948559 | 43847 | 8:85052405 | 43927 | 8:87126956 | 43963 | 8:87960883 | 43972 | 8:88206973 |
| 43973 | 8:88207717 | 44068 | 8:90411690 | 44089 | 8:90899216 | 44097 | 8:91046537 | 44301 | 8:96913052 |
| 44325 | 8:97476895 | 44365 | 8:98665857 | 44390 | 8:99176725 | 44398 | 8:99311311 | 44414 | 8:99547619 |
| 44418 | 8:99577548 | 44438 | 8:100015023 | 44490 | 8:101376581 | 44635 | 8:103813682 | 44653 | 8:104231786 |
| 44746 | 8:106532357 | 44749 | 8:106594158 | 44792 | 8:107582608 | 45042 | 9:2688546 | 45058 | 9:3203477 |
| 45130 | 9:5470319 | 45151 | 9:6116416 | 45173 | 9:6877480 | 45177 | 9:7031515 | 45229 | 9:8388451 |
| 45283 | 9:9884501 | 45296 | 9:10301651 | 45298 | 9:10310168 | 45417 | 9:11927500 | 45439 | 9:12374207 |
| 45514 | 9:13742514 | 45529 | 9:14218593 | 45532 | 9:14239370 | 45566 | 9:15019469 | 45593 | 9:15465522 |
| 45684 | 9:17366049 | 45686 | 9:17428879 | 45709 | 9:18143920 | 45711 | 9:18153000 | 45712 | 9:18160118 |
| 45757 | 9:19618032 | 45977 | 9:25228881 | 45987 | 9:25512136 | 46000 | 9:25838484 | 46031 | 9:26618713 |
| 46034 | 9:26691579 | 46063 | 9:27473901 | 46065 | 9:27477523 | 46066 | 9:27485623 | 46095 | 9:28146296 |
| 46123 | 9:28679350 | 46135 | 9:28927068 | 46242 | 9:30976439 | 46245 | 9:31003562 | 46276 | 9:31748693 |
| 46330 | 9:33062497 | 46343 | 9:33376755 | 46364 | 9:33886935 | 46372 | 9:34155670 | 46516 | 9:37778578 |
| 46545 | 9:38309457 | 46546 | 9:38314365 | 46561 | 9:38550987 | 46644 | 9:40310425 | 46767 | 9:43367400 |
| 46771 | 9:43430747 | 46802 | 9:44301570 | 46864 | 9:46172575 | 46865 | 9:46201814 | 46916 | 9:47818319 |
| 46943 | 9:48865487 | 46946 | 9:48902577 | 46963 | 9:49476706 | 46974 | 9:49653803 | 47048 | 9:51753467 |
| 47067 | 9:52274853 | 47097 | 9:52875308 | 47148 | 9:54342081 | 47149 | 9:54361703 | 47168 | 9:54807613 |
| 47325 | 9:59403628 | 47340 | 9:59637965 | 47341 | 9:59638253 | 47342 | 9:59641031 | 47343 | 9:59643481 |
| 47344 | 9:59645062 | 47345 | 9:59645804 | 47346 | 9:59647105 | 47347 | 9:59650376 | 47349 | 9:59653721 |
| 47385 | 9:60709648 | 47395 | 9:60924554 | 47396 | 9:60926410 | 47425 | 9:61795735 | 47611 | 9:65961632 |
| 47667 | 9:67443259 | 47669 | 9:67511922 | 47679 | 9:67708264 | 47688 | 9:68029230 | 47727 | 9:69025344 |
| 47770 | 9:70119558 | 47782 | 9:70360272 | 47843 | 9:71633299 | 47985 | 9:75491653 | 48031 | 9:76631634 |
| 48186 | 9:79336612 | 48219 | 9:80142294 | 48257 | 9:80938431 | 48267 | 9:81108364 | 48274 | 9:81217242 |
| 48305 | 9:81845883 | 48312 | 9:82017648 | 48340 | 9:82626166 | 48342 | 9:82647502 | 48343 | 9:82651491 |
| 48345 | 9:82668654 | 48348 | 9:82682681 | 48354 | 9:82701768 | 48361 | 9:82842810 | 48394 | 9:83693006 |
| 48397 | 9:83826419 | 48468 | 9:85456175 | 48490 | 9:85784712 | 48550 | 9:87315819 | 48553 | 9:87327271 |
| 48558 | 9:87361936 | 48559 | 9:87373306 | 48590 | 9:87819557 | 48591 | 9:87822671 | 48627 | 9:88634584 |
| 48631 | 9:88656403 | 48632 | 9:88680123 | 48707 | 9:89908123 | 48708 | 9:89918140 | 48710 | 9:89921487 |
| 48713 | 9:90012380 | 48748 | 9:90826907 | 48780 | 9:91531504 | 48810 | 9:92215180 | 48815 | 9:92295269 |
| 48856 | 9:93221725 | 48880 | 9:93749307 | 48891 | 9:93889254 | 48911 | 9:94252780 | 48914 | 9:94269583 |
| 48923 | 9:94459307 | 48924 | 9:94493145 | 48986 | 9:95568256 | 49048 | 9:96604431 | 49052 | 9:96651231 |
| 49053 | 9:96652474 | 49054 | 9:96655491 | 49055 | 9:96658405 | 49056 | 9:96659811 | 49059 | 9:96727179 |
| 49060 | 9:96730432 | 49065 | 9:96818068 | 49100 | 9:97292214 | 49186 | 9:99056298 | 49199 | 9:99304134 |
| 49204 | 9:99370466 | 49267 | 9:100494397 | 49269 | 9:100511637 | 49277 | 9:100658454 | 49280 | 9:100721863 |
| 49342 | 9:101817009 | 49343 | 9:101818874 | 49344 | 9:101822513 | 49346 | 9:101827562 | 49349 | 9:101865213 |
| 49442 | 9:103263715 | 49469 | 9:103800979 | 49529 | 10:872754 | 49531 | 10:882204 | 49542 | 10:1178970 |
| 49569 | 10:1795897 | 49614 | 10:2626851 | 49654 | 10:3355041 | 49730 | 10:4924755 | 49746 | 10:5330263 |
| 49804 | 10:6194603 | 49811 | 10:6389416 | 49855 | 10:7506260 | 49856 | 10:7508517 | 49896 | 10:8296974 |
| 49909 | 10:8505165 | 49939 | 10:9303914 | 49984 | 10:10211405 | 49995 | 10:10425877 | 50019 | 10:10791977 |
| 50022 | 10:10887987 | 50067 | 10:11918946 | 50097 | 10:12461852 | 50162 | 10:13597734 | 50172 | 10:13748622 |
| 50235 | 10:15125993 | 50326 | 10:16719617 | 50347 | 10:17070966 | 50355 | 10:17235899 | 50385 | 10:17893631 |
| 50510 | 10:20312528 | 50512 | 10:20320283 | 50597 | 10:21831782 | 50627 | 10:22355891 | 50730 | 10:26724399 |
| 50740 | 10:26816725 | 50748 | 10:26936267 | 50763 | 10:27119455 | 50820 | 10:28726853 | 50827 | 10:28818762 |
| 50840 | 10:28923770 | 50844 | 10:28933854 | 50884 | 10:29514735 | 50885 | 10:29522253 | 50886 | 10:29533360 |
| 50906 | 10:30075494 | 50909 | 10:30126897 | 50911 | 10:30167171 | 50955 | 10:31703939 | 50968 | 10:32063661 |
| 50983 | 10:32536622 | 51010 | 10:33047373 | 51015 | 10:33221086 | 51073 | 10:34533777 | 51093 | 10:34950052 |
| 51125 | 10:35727830 | 51189 | 10:37345559 | 51190 | 10:37377718 | 51191 | 10:37388003 | 51202 | 10:37683947 |
| 51210 | 10:37775786 | 51223 | 10:38166114 | 51227 | 10:38269447 | 51243 | 10:38653361 | 51265 | 10:39263333 |
| 51295 | 10:40078678 | 51332 | 10:41115727 | 51348 | 10:41693430 | 51354 | 10:41758465 | 51445 | 10:44433737 |
| 51454 | 10:44645299 | 51494 | 10:45580212 | 51611 | 10:47920359 | 51626 | 10:48320530 | 51643 | 10:48547551 |
| 51688 | 10:49516649 | 51721 | 10:50223805 | 51725 | 10:50334170 | 51759 | 10:51169084 | 51760 | 10:51181102 |
| 51761 | 10:51186753 | 51773 | 10:51492328 | 51784 | 10:51754878 | 51809 | 10:52286224 | 51840 | 10:52879725 |
| 51889 | 10:54463903 | 51952 | 10:56290718 | 51963 | 10:56443542 | 52006 | 10:57713193 | 52037 | 10:58360318 |
| 52049 | 10:58658013 | 52120 | 10:61117665 | 52181 | 10:62385028 | 52245 | 10:64094738 | 52272 | 10:64614710 |
| 52276 | 10:64656067 | 52345 | 10:66304541 | 52390 | 10:67276588 | 52391 | 10:67310241 | 52399 | 10:67673108 |
| 52467 | 10:68971489 | 52480 | 10:69334027 | 52486 | 10:69501262 | 52499 | 10:69823517 | 52501 | 10:69836682 |
| 52504 | 10:69941547 | 52513 | 10:70197389 | 52537 | 10:71023784 | 52552 | 10:71449748 | 52570 | 10:72023102 |
| 52598 | 10:73101962 | 52623 | 10:73660537 | 52653 | 10:74459889 | 52665 | 10:75024558 | 52672 | 10:75197354 |
| 52688 | 10:75607741 | 52743 | 10:76813636 | 52823 | 10:78537516 | 52826 | 10:78594348 | 52840 | 10:79035728 |
| 52900 | 10:80684424 | 52913 | 10:81068664 | 52970 | 10:81760396 | 53006 | 10:82276845 | 53023 | 10:82877025 |
| 53054 | 10:83522156 | 53055 | 10:83550757 | 53062 | 10:83712797 | 53068 | 10:83857093 | 53071 | 10:83936676 |
| 53099 | 10:84417728 | 53239 | 10:87507691 | 53287 | 10:88767077 | 53305 | 10:89008507 | 53312 | 10:89144342 |
| 53340 | 10:89729222 | 53366 | 10:90207696 | 53388 | 10:90615371 | 53487 | 10:92661034 | 53497 | 10:92892557 |
| 53545 | 10:94160502 | 53555 | 10:94348103 | 53604 | 10:95590054 | 53623 | 10:95920911 | 53627 | 10:96023548 |
| 53669 | 10:97104354 | 53675 | 10:97169425 | 53745 | 10:98522222 | 53797 | 10:99634106 | 53869 | 10:101120062 |
| 53875 | 10:101177760 | 53882 | 10:101281677 | 53887 | 10:101426015 | 53913 | 10:101819543 | 53944 | 10:102268731 |
| 54003 | 11:441012 | 54045 | 11:1204347 | 54069 | 11:1549638 | 54111 | 11:1989902 | 54174 | 11:2689033 |
| 54209 | 11:3235907 | 54259 | 11:4054643 | 54292 | 11:4984752 | 54300 | 11:5106453 | 54327 | 11:5677169 |
| 54409 | 11:7670104 | 54425 | 11:8021628 | 54476 | 11:9219377 | 54572 | 11:11342389 | 54630 | 11:12715868 |
| 54638 | 11:12870807 | 54674 | 11:13642677 | 54677 | 11:13704625 | 54736 | 11:15343003 | 54745 | 11:15626371 |
| 54747 | 11:15658858 | 54756 | 11:15853957 | 54797 | 11:16775998 | 54856 | 11:18554101 | 54872 | 11:18844419 |
| 54875 | 11:18948217 | 54883 | 11:19173839 | 54923 | 11:20562842 | 54924 | 11:20568845 | 54981 | 11:21832414 |
| 55037 | 11:23157077 | 55048 | 11:23494343 | 55084 | 11:24320520 | 55092 | 11:24369984 | 55093 | 11:24377983 |
| 55094 | 11:24393545 | 55096 | 11:24408850 | 55119 | 11:24944857 | 55151 | 11:25930515 | 55161 | 11:26154577 |
| 55174 | 11:26362493 | 55214 | 11:27414983 | 55238 | 11:27959917 | 55283 | 11:28844947 | 55302 | 11:29230653 |
| 55315 | 11:29502309 | 55352 | 11:30379793 | 55380 | 11:30989937 | 55381 | 11:31003265 | 55382 | 11:31020736 |
| 55383 | 11:31023833 | 55425 | 11:32229026 | 55458 | 11:33116089 | 55479 | 11:33643610 | 55496 | 11:34099362 |
| 55532 | 11:35548594 | 55538 | 11:35660110 | 55568 | 11:36429662 | 55588 | 11:37003872 | 55608 | 11:37326478 |
| 55615 | 11:37508364 | 55620 | 11:37570757 | 55624 | 11:37639983 | 55768 | 11:41573665 | 55824 | 11:42850632 |
| 55835 | 11:43167995 | 55873 | 11:44279092 | 55880 | 11:44503647 | 55915 | 11:45256103 | 55925 | 11:45835142 |
| 55933 | 11:45970733 | 55938 | 11:46039317 | 55941 | 11:46045608 | 55942 | 11:46058932 | 55972 | 11:46675342 |
| 56017 | 11:47326731 | 56043 | 11:47992234 | 56067 | 11:48522658 | 56126 | 11:49051072 | 56190 | 11:50710309 |
| 56222 | 11:51848453 | 56258 | 11:53253320 | 56273 | 11:53826835 | 56332 | 11:55052140 | 56335 | 11:55137862 |
| 56337 | 11:55149593 | 56397 | 11:56859473 | 56653 | 11:63292715 | 56682 | 11:63832921 | 56683 | 11:63837249 |
| 56714 | 11:64511310 | 56734 | 11:65119714 | 56739 | 11:65250246 | 56752 | 11:65585594 | 56764 | 11:65826676 |
| 56767 | 11:66038610 | 56784 | 11:66319597 | 56812 | 11:67082495 | 56896 | 11:68461614 | 56913 | 11:68937779 |
| 56917 | 11:69060122 | 56924 | 11:69109200 | 56925 | 11:69118745 | 56928 | 11:69142947 | 56933 | 11:69181613 |
| 56943 | 11:69335989 | 57008 | 11:70675473 | 57082 | 11:71401956 | 57156 | 11:73126654 | 57200 | 11:74123227 |
| 57271 | 11:75902686 | 57350 | 11:77659975 | 57451 | 11:79225059 | 57506 | 11:80496876 | 57543 | 11:81586331 |
| 57554 | 11:81811191 | 57559 | 11:81888932 | 57573 | 11:82234600 | 57724 | 11:85632013 | 57806 | 11:87517972 |
| 57868 | 11:88067171 | 57900 | 11:88940240 | 57915 | 11:89270730 | 57969 | 11:90490496 | 57973 | 11:90559289 |
| 57995 | 11:91106611 | 58020 | 11:91612293 | 58045 | 11:92156116 | 58120 | 11:93813789 | 58133 | 11:94097451 |
| 58153 | 11:94563151 | 58180 | 11:95385268 | 58195 | 11:95727670 | 58219 | 11:96378020 | 58238 | 11:97095593 |
| 58246 | 11:97197864 | 58308 | 11:98538984 | 58364 | 11:99473375 | 58417 | 11:100532361 | 58536 | 11:102829142 |
| 58591 | 11:103534827 | 58607 | 11:104097856 | 58720 | 11:106485352 | 58877 | 12:3502498 | 58943 | 12:5186588 |
| 58945 | 12:5325638 | 58966 | 12:6004879 | 58987 | 12:6773018 | 59008 | 12:7507940 | 59043 | 12:8830007 |
| 59057 | 12:9272154 | 59061 | 12:9329563 | 59069 | 12:9522013 | 59119 | 12:10577179 | 59120 | 12:10585260 |
| 59155 | 12:11315928 | 59176 | 12:11720838 | 59179 | 12:11790945 | 59182 | 12:11827023 | 59184 | 12:11857024 |
| 59208 | 12:12366690 | 59238 | 12:13384011 | 59260 | 12:13823314 | 59276 | 12:14077064 | 59282 | 12:14150866 |
| 59301 | 12:14486563 | 59316 | 12:14918565 | 59397 | 12:16476525 | 59417 | 12:16974977 | 59428 | 12:17253883 |
| 59434 | 12:17359694 | 59439 | 12:17465924 | 59454 | 12:17933719 | 59471 | 12:18284739 | 59549 | 12:20109323 |
| 59698 | 12:23940231 | 59702 | 12:24068026 | 59727 | 12:24577728 | 59786 | 12:25839029 | 59799 | 12:26457768 |
| 59840 | 12:27377754 | 59846 | 12:27488955 | 59870 | 12:28122480 | 59956 | 12:30649835 | 59989 | 12:31264310 |
| 60008 | 12:31780482 | 60117 | 12:34750935 | 60139 | 12:35299411 | 60141 | 12:35311925 | 60142 | 12:35312992 |
| 60143 | 12:35314580 | 60144 | 12:35321842 | 60145 | 12:35326856 | 60147 | 12:35334692 | 60148 | 12:35339381 |
| 60149 | 12:35342141 | 60151 | 12:35355118 | 60152 | 12:35355785 | 60153 | 12:35363362 | 60154 | 12:35424126 |
| 60156 | 12:35487159 | 60202 | 12:37059151 | 60206 | 12:37121439 | 60347 | 12:42536086 | 60352 | 12:42838412 |
| 60389 | 12:44505965 | 60458 | 12:46806766 | 60555 | 12:49288668 | 60578 | 12:49872127 | 60583 | 12:49903248 |
| 60596 | 12:50116234 | 60604 | 12:50253408 | 60730 | 12:53098596 | 60840 | 12:55588645 | 60862 | 12:56409050 |
| 60875 | 12:56736797 | 60935 | 12:58879036 | 61043 | 12:62502730 | 61081 | 12:63602083 | 61084 | 12:63713299 |
| 61163 | 12:65908229 | 61179 | 12:66475638 | 61325 | 12:69535302 | 61424 | 12:73231474 | 61483 | 12:74215241 |
| 61488 | 12:74344277 | 61565 | 12:76057266 | 61574 | 12:76294965 | 61575 | 12:76298488 | 61597 | 12:76881665 |
| 61771 | 12:80709446 | 61777 | 12:80884100 | 61814 | 12:81734916 | 61831 | 12:82087879 | 61844 | 12:82441644 |
| 61856 | 12:82591128 | 61912 | 12:83854133 | 61961 | 12:84933491 | 61993 | 12:85357065 | 62129 | 13:1206621 |
| 62198 | 13:2453852 | 62215 | 13:2711565 | 62223 | 13:2835285 | 62289 | 13:4735864 | 62347 | 13:5936143 |
| 62362 | 13:6285905 | 62525 | 13:10419094 | 62677 | 13:14935831 | 62696 | 13:15681048 | 62771 | 13:17258775 |
| 62815 | 13:17832527 | 62916 | 13:19454627 | 62947 | 13:19907032 | 62968 | 13:20390279 | 62969 | 13:20405413 |
| 62970 | 13:20411494 | 62971 | 13:20423875 | 62972 | 13:20430234 | 62988 | 13:20701775 | 63002 | 13:21060977 |
| 63055 | 13:22024146 | 63112 | 13:23989482 | 63141 | 13:24644133 | 63143 | 13:24667549 | 63150 | 13:24857890 |
| 63174 | 13:25338856 | 63181 | 13:25455632 | 63193 | 13:25724492 | 63199 | 13:25901617 | 63240 | 13:27082022 |
| 63282 | 13:27909495 | 63285 | 13:28007570 | 63295 | 13:28335861 | 63305 | 13:28545136 | 63341 | 13:29355083 |
| 63359 | 13:29815935 | 63424 | 13:31076841 | 63427 | 13:31114025 | 63436 | 13:31276115 | 63470 | 13:31684931 |
| 63484 | 13:32188070 | 63485 | 13:32192701 | 63486 | 13:32195543 | 63488 | 13:32202239 | 63490 | 13:32214896 |
| 63507 | 13:32509667 | 63513 | 13:32595507 | 63515 | 13:32606184 | 63519 | 13:32649822 | 63520 | 13:32651100 |
| 63644 | 13:35586171 | 63673 | 13:36380278 | 63730 | 13:37696035 | 63799 | 13:39119033 | 63842 | 13:39974325 |
| 63843 | 13:40001169 | 63848 | 13:40028952 | 63884 | 13:40680902 | 63923 | 13:41808005 | 63934 | 13:41965173 |
| 63957 | 13:42527459 | 64015 | 13:43212135 | 64028 | 13:43604201 | 64041 | 13:43854540 | 64042 | 13:43855779 |
| 64043 | 13:43868655 | 64044 | 13:43869987 | 64045 | 13:43870636 | 64046 | 13:43872533 | 64047 | 13:43873040 |
| 64048 | 13:43873873 | 64049 | 13:43876772 | 64050 | 13:43878363 | 64051 | 13:43879388 | 64052 | 13:43880125 |
| 64053 | 13:43884214 | 64258 | 13:47680398 | 64280 | 13:48210593 | 64321 | 13:49204706 | 64383 | 13:51177851 |
| 64397 | 13:51341948 | 64439 | 13:52703815 | 64510 | 13:53977365 | 64538 | 13:54845724 | 64978 | 13:64716430 |
| 64982 | 13:64796939 | 64998 | 13:65331493 | 65047 | 13:66193660 | 65061 | 13:66407606 | 65093 | 13:67181101 |
| 65096 | 13:67321578 | 65215 | 13:70526426 | 65221 | 13:70575324 | 65237 | 13:70871738 | 65271 | 13:71637090 |
| 65328 | 13:72957542 | 65357 | 13:73471150 | 65381 | 13:73812183 | 65456 | 13:75531070 | 65529 | 13:76798256 |
| 65547 | 13:77244637 | 65550 | 13:77279091 | 65578 | 13:77845959 | 65598 | 13:78300577 | 65660 | 13:79330804 |
| 65677 | 13:79542295 | 65819 | 13:82171173 | 65917 | 14:1204169 | 65929 | 14:1645654 | 66049 | 14:3651551 |
| 66072 | 14:4085451 | 66078 | 14:4200331 | 66116 | 14:4961976 | 66160 | 14:5700495 | 66209 | 14:6316411 |
| 66268 | 14:7449642 | 66271 | 14:7527096 | 66317 | 14:8213069 | 66475 | 14:10912061 | 66523 | 14:11744884 |
| 66538 | 14:12080094 | 66544 | 14:12195646 | 66565 | 14:12541653 | 66566 | 14:12551654 | 66568 | 14:12570995 |
| 66627 | 14:14043986 | 66718 | 14:16010978 | 66719 | 14:16022809 | 66722 | 14:16058309 | 66732 | 14:16280608 |
| 66746 | 14:16568761 | 66820 | 14:18106996 | 66821 | 14:18115961 | 66855 | 14:19074429 | 66858 | 14:19172311 |
| 66877 | 14:19623114 | 66903 | 14:20293557 | 66912 | 14:20629249 | 66916 | 14:20662609 | 66917 | 14:20673221 |
| 66925 | 14:20792640 | 67108 | 14:22916046 | 67140 | 14:22986080 | 67146 | 14:22999212 | 67302 | 14:23338890 |
| 67351 | 14:23565143 | 67354 | 14:23600267 | 67355 | 14:23607944 | 67357 | 14:23630896 | 67359 | 14:23652804 |
| 67369 | 14:23826318 | 67371 | 14:23829066 | 67374 | 14:23831133 | 67501 | 14:24941221 | 67613 | 14:26283998 |
| 67660 | 14:27607287 | 67681 | 14:28330433 | 67709 | 14:29096850 | 67747 | 14:29873926 | 67772 | 14:30389540 |
| 67822 | 14:31570898 | 67845 | 14:32267064 | 67901 | 14:33278402 | 67955 | 14:34370911 | 68032 | 14:36313786 |
| 68053 | 14:36889990 | 68099 | 14:38260754 | 68128 | 14:39143238 | 68172 | 14:40259820 | 68190 | 14:40976286 |
| 68197 | 14:41229576 | 68198 | 14:41237524 | 68261 | 14:42746359 | 68276 | 14:43241727 | 68313 | 14:44566510 |
| 68321 | 14:44666155 | 68354 | 14:45237526 | 68360 | 14:45367206 | 68364 | 14:45505076 | 68368 | 14:45610432 |
| 68441 | 14:46995812 | 68448 | 14:47163738 | 68460 | 14:47428677 | 68478 | 14:48150783 | 68509 | 14:48767270 |
| 68536 | 14:49487643 | 68580 | 14:50970280 | 68581 | 14:50986924 | 68606 | 14:51659332 | 68620 | 14:51985037 |
| 68654 | 14:52815945 | 68668 | 14:53442111 | 68687 | 14:54022606 | 68730 | 14:54568169 | 68772 | 14:55536222 |
| 68826 | 14:57247647 | 68861 | 14:57991139 | 68870 | 14:58182404 | 68871 | 14:58201975 | 68907 | 14:58964090 |
| 68974 | 14:60591022 | 68975 | 14:60610629 | 68988 | 14:60975742 | 69085 | 14:62782594 | 69115 | 14:63450732 |
| 69130 | 14:63928126 | 69158 | 14:64538337 | 69184 | 14:65420223 | 69193 | 14:65688670 | 69200 | 14:65782382 |
| 69231 | 14:66405235 | 69239 | 14:66655367 | 69251 | 14:66916531 | 69316 | 14:68245567 | 69466 | 14:72276625 |
| 69482 | 14:72695548 | 69536 | 14:73988875 | 69639 | 14:76386785 | 69745 | 14:78754068 | 69764 | 14:79462729 |
| 69867 | 14:81514348 | 69890 | 14:81754335 | 69891 | 14:81755109 | 69932 | 15:549186 | 69952 | 15:1223740 |
| 69971 | 15:1763083 | 70074 | 15:4197179 | 70097 | 15:4603953 | 70120 | 15:5062052 | 70314 | 15:6267325 |
| 70382 | 15:7840165 | 70418 | 15:8668231 | 70426 | 15:8980586 | 70486 | 15:10462503 | 70525 | 15:11922022 |
| 70590 | 15:14196834 | 70637 | 15:15315326 | 70696 | 15:16104588 | 70710 | 15:16454917 | 70736 | 15:17101878 |
| 70782 | 15:18196851 | 70794 | 15:18357447 | 70804 | 15:18736931 | 70814 | 15:19007825 | 70838 | 15:19653453 |
| 70946 | 15:22147187 | 70997 | 15:23248430 | 71016 | 15:23621459 | 71018 | 15:23665519 | 71079 | 15:24372603 |
| 71091 | 15:24613968 | 71107 | 15:24837501 | 71123 | 15:25257089 | 71128 | 15:25308638 | 71168 | 15:26173379 |
| 71188 | 15:26573138 | 71308 | 15:28904879 | 71323 | 15:29119476 | 71357 | 15:30139511 | 71368 | 15:30396261 |
| 71408 | 15:31365259 | 71434 | 15:31780017 | 71443 | 15:32064162 | 71478 | 15:32883165 | 71517 | 15:33758501 |
| 71518 | 15:33767591 | 71580 | 15:34828367 | 71614 | 15:35688175 | 71626 | 15:35959116 | 71745 | 15:38587541 |
| 71763 | 15:38952840 | 71840 | 15:40889581 | 71862 | 15:41323112 | 71923 | 15:42472180 | 71943 | 15:42904887 |
| 72011 | 15:43795583 | 72016 | 15:43848104 | 72053 | 15:44559517 | 72177 | 15:46955071 | 72194 | 15:47343068 |
| 72237 | 15:48145511 | 72241 | 15:48207972 | 72244 | 15:48254047 | 72281 | 15:49497449 | 72308 | 15:50463550 |
| 72408 | 15:52214824 | 72429 | 15:52810027 | 72524 | 15:54359875 | 72578 | 15:55624075 | 72586 | 15:55875613 |
| 72693 | 15:57504930 | 72717 | 15:57932533 | 72747 | 15:58462557 | 72792 | 15:59699546 | 72793 | 15:59714205 |
| 72835 | 15:60939376 | 72865 | 15:61561772 | 72880 | 15:61802396 | 72881 | 15:61805665 | 72882 | 15:61806975 |
| 72885 | 15:61812433 | 72890 | 15:61960293 | 72891 | 15:61964427 | 72930 | 15:62856332 | 73076 | 15:65465351 |
| 73108 | 15:66183419 | 73192 | 15:68248786 | 73261 | 15:70440283 | 73286 | 15:71178088 | 73301 | 15:71472435 |
| 73332 | 15:72309114 | 73377 | 15:73291708 | 73393 | 15:73582243 | 73416 | 15:74163082 | 73451 | 15:74779756 |
| 73466 | 15:75089743 | 73510 | 15:76195722 | 73541 | 15:76835072 | 73630 | 15:78278239 | 73698 | 15:80309655 |
| 73754 | 15:81295713 | 73762 | 15:81438153 | 73813 | 15:82751747 | 73823 | 15:82918904 | 73880 | 15:83865721 |
| 73959 | 16:1501024 | 74086 | 16:4079477 | 7408 | 16:4114086 | 74111 | 16:4443184 | 74212 | 16:7478039 |
| 74284 | 16:10121160 | 74304 | 16:10857824 | 74327 | 16:11521662 | 74329 | 16:11621503 | 74349 | 16:12056975 |
| 74446 | 16:14409239 | 74451 | 16:14518175 | 74462 | 16:14841844 | 74479 | 16:15208552 | 74481 | 16:15279430 |
| 74498 | 16:15816016 | 74506 | 16:16013694 | 74511 | 16:16188270 | 74513 | 16:16194660 | 74578 | 16:18520385 |
| 74584 | 16:18637360 | 74591 | 16:18804747 | 74652 | 16:20427119 | 74853 | 16:25049887 | 74887 | 16:25231259 |
| 74902 | 16:25476855 | 74955 | 16:26802374 | 74956 | 16:26859375 | 74969 | 16:27035419 | 75000 | 16:27937410 |
| 75006 | 16:28023322 | 75055 | 16:29594638 | 75144 | 16:31915038 | 75148 | 16:31970232 | 75161 | 16:32424717 |
| 75174 | 16:32836939 | 75253 | 16:34714234 | 75284 | 16:35365207 | 75318 | 16:36337580 | 75342 | 16:36921696 |
| 75354 | 16:37184848 | 75457 | 16:39917217 | 75492 | 16:41063333 | 75508 | 16:41613390 | 75547 | 16:42048065 |
| 75564 | 16:42830906 | 75611 | 16:44815088 | 75636 | 16:45912914 | 75654 | 16:46353096 | 75728 | 16:48055006 |
| 75756 | 16:48627494 | 75757 | 16:48635114 | 75758 | 16:48637200 | 75759 | 16:48639734 | 75760 | 16:48645579 |
| 75761 | 16:48649527 | 75825 | 16:50275447 | 75834 | 16:50665850 | 75880 | 16:51861172 | 75887 | 16:52014418 |
| 75915 | 16:52578534 | 75975 | 16:54055090 | 75981 | 16:54147078 | 76006 | 16:54922205 | 76068 | 16:56374651 |
| 76087 | 16:56788303 | 76136 | 16:58112073 | 76149 | 16:58378141 | 76153 | 16:58406289 | 76198 | 16:59309602 |
| 76229 | 16:60126861 | 76246 | 16:60365696 | 76276 | 16:61171484 | 76282 | 16:61360408 | 76298 | 16:61585266 |
| 76320 | 16:62102938 | 76325 | 16:62226456 | 76366 | 16:63181951 | 76484 | 16:65323416 | 76487 | 16:65420109 |
| 76600 | 16:67626229 | 76631 | 16:68532430 | 76672 | 16:69359404 | 76687 | 16:69663665 | 76741 | 16:70858584 |
| 76747 | 16:70938743 | 76846 | 16:73114325 | 76890 | 16:73720962 | 76916 | 16:74372695 | 76921 | 16:74515053 |
| 77113 | 16:78293826 | 77116 | 16:78337900 | 77145 | 16:78921036 | 77151 | 16:78991296 | 77156 | 16:79028746 |
| 77158 | 16:79043302 | 77226 | 16:79983101 | 77237 | 16:80656680 | 77361 | 17:3014326 | 77369 | 17:3204260 |
| 77405 | 17:3947723 | 77459 | 17:4815417 | 77461 | 17:4875306 | 77475 | 17:5216472 | 77564 | 17:7040082 |
| 77625 | 17:8272941 | 77711 | 17:10199997 | 77712 | 17:10213106 | 78005 | 17:17163300 | 78016 | 17:17334505 |
| 78018 | 17:17357791 | 78027 | 17:17451464 | 78083 | 17:18818415 | 78126 | 17:19479964 | 78154 | 17:20220411 |
| 78164 | 17:20579995 | 78176 | 17:21008303 | 78218 | 17:22333330 | 78297 | 17:25079038 | 78310 | 17:25505355 |
| 78350 | 17:26610845 | 78351 | 17:26613473 | 78352 | 17:26635840 | 78357 | 17:26689830 | 78358 | 17:26694252 |
| 78359 | 17:26696213 | 78360 | 17:26697673 | 78362 | 17:26701612 | 78363 | 17:26731287 | 78365 | 17:26786597 |
| 78434 | 17:28547562 | 78459 | 17:29295556 | 78584 | 17:32536552 | 78587 | 17:32649823 | 78592 | 17:32740716 |
| 78597 | 17:32819809 | 78603 | 17:32962598 | 78617 | 17:33367107 | 78663 | 17:34481729 | 78680 | 17:34921588 |
| 78756 | 17:37414365 | 78774 | 17:38086662 | 78821 | 17:39604254 | 78835 | 17:39829382 | 78858 | 17:40028314 |
| 78866 | 17:40286179 | 78900 | 17:40901144 | 78909 | 17:41080474 | 78952 | 17:42386980 | 79026 | 17:44070161 |
| 79035 | 17:44280518 | 79050 | 17:44811665 | 79055 | 17:44886429 | 79088 | 17:45543757 | 79096 | 17:45774161 |
| 79102 | 17:45892895 | 79105 | 17:45908272 | 79147 | 17:46796152 | 79149 | 17:46809775 | 79150 | 17:46813078 |
| 79151 | 17:46817568 | 79152 | 17:46826871 | 79165 | 17:47298668 | 79168 | 17:47374048 | 79198 | 17:48075712 |
| 79217 | 17:48705395 | 79225 | 17:48883686 | 79261 | 17:49706199 | 79286 | 17:50270053 | 79354 | 17:51608281 |
| 79389 | 17:52115500 | 79391 | 17:52143477 | 79448 | 17:53925709 | 79467 | 17:54247909 | 79477 | 17:54494775 |
| 79546 | 17:56254970 | 79547 | 17:56259657 | 79577 | 17:57167393 | 79593 | 17:57507972 | 79621 | 17:57996907 |
| 79692 | 17:59257237 | 79708 | 17:59553144 | 79724 | 17:59967182 | 79763 | 17:60561298 | 79767 | 17:60653296 |
| 79776 | 17:60751860 | 79777 | 17:60776828 | 79791 | 17:60997432 | 79792 | 17:61002338 | 79793 | 17:61005235 |
| 79794 | 17:61005949 | 79799 | 17:61043189 | 79800 | 17:61068599 | 79804 | 17:61124602 | 79805 | 17:61125930 |
| 79806 | 17:61128578 | 79809 | 17:61149706 | 79811 | 17:61202835 | 79891 | 17:63194677 | 79894 | 17:63214725 |
| 79897 | 17:63260940 | 79924 | 17:63621804 | 79982 | 17:64851883 | 80057 | 17:66198895 | 80059 | 17:66227047 |
| 80104 | 17:67019380 | 80140 | 17:68128330 | 80158 | 17:68440573 | 80180 | 17:68951402 | 80183 | 17:68969488 |
| 80198 | 17:69224867 | 80234 | 17:70077612 | 80285 | 17:70987613 | 80306 | 17:71441658 | 80354 | 17:72302909 |
| 80450 | 18:1522517 | 80502 | 18:1802503 | 80508 | 18:1955689 | 80546 | 18:2744105 | 80574 | 18:3295457 |
| 80617 | 18:4333966 | 80629 | 18:4581539 | 80638 | 18:4763164 | 80660 | 18:5336542 | 80662 | 18:5358167 |
| 80679 | 18:5624880 | 80687 | 18:5808571 | 80698 | 18:6108723 | 80706 | 18:6166618 | 80715 | 18:6297029 |
| 80718 | 18:6328373 | 80801 | 18:7890552 | 80807 | 18:8137616 | 80811 | 18:8163590 | 80812 | 18:8166965 |
| 80813 | 18:8169505 | 80832 | 18:8512615 | 80833 | 18:8514080 | 80853 | 18:8977357 | 80855 | 18:9002587 |
| 80856 | 18:9003665 | 80857 | 18:9005002 | 80860 | 18:9032209 | 80867 | 18:9092871 | 80870 | 18:9135683 |
| 80881 | 18:9393342 | 80892 | 18:9513766 | 80943 | 18:10501656 | 80948 | 18:10593010 | 80951 | 18:10632938 |
| 80976 | 18:11155672 | 81045 | 18:12950981 | 81047 | 18:12967212 | 81076 | 18:13742613 | 81096 | 18:14219190 |
| 81124 | 18:15295023 | 81131 | 18:15355195 | 81156 | 18:16027392 | 81169 | 18:16320799 | 81178 | 18:16458903 |
| 81221 | 18:17381122 | 81234 | 18:17689118 | 81235 | 18:17739758 | 81305 | 18:19009400 | 81313 | 18:19119273 |
| 81333 | 18:19791460 | 81431 | 18:21965199 | 81444 | 18:22114555 | 81457 | 18:22311504 | 81475 | 18:22771950 |
| 81480 | 18:22863942 | 81493 | 18:23128319 | 81518 | 18:23726377 | 81540 | 18:24231814 | 81553 | 18:24599361 |
| 81590 | 18:25135273 | 81669 | 18:26830057 | 81689 | 18:27317835 | 81727 | 18:28307360 | 81772 | 18:29581458 |
| 81773 | 18:29587945 | 81783 | 18:29882243 | 81793 | 18:30199537 | 81805 | 18:30616085 | 81868 | 18:32286982 |
| 81873 | 18:32423923 | 81905 | 18:33102972 | 81907 | 18:33133073 | 81990 | 18:35092205 | 82005 | 18:35445465 |
| 82073 | 18:36962507 | 82091 | 18:37442129 | 82129 | 18:38169007 | 82139 | 18:38310010 | 82172 | 18:39170079 |
| 82266 | 18:41092657 | 82284 | 18:41476993 | 82310 | 18:42077536 | 82369 | 18:43146823 | 82379 | 18:43316323 |
| 82386 | 18:43394501 | 82412 | 18:43955990 | 82472 | 18:45795414 | 82478 | 18:45922293 | 82497 | 18:46310174 |
| 82503 | 18:46383891 | 82580 | 18:46784427 | 82591 | 18:46951310 | 82605 | 18:47321091 | 82699 | 18:49081014 |
| 82702 | 18:49135986 | 82724 | 18:49817333 | 82819 | 18:51867088 | 82926 | 18:54273698 | 82986 | 18:55259160 |
| 83048 | 18:56589786 | 83125 | 18:57434771 | 83138 | 18:57567593 | 83261 | 18:60011920 | 83313 | 18:61667250 |
| 83390 | 18:63188823 | 83451 | 18:64129214 | 83452 | 18:64134704 | 83491 | 18:64654290 | 83495 | 18:64731877 |
| 83505 | 18:64973481 | 83524 | 18:65148413 | 83569 | 18:65462851 | 83715 | 19:4421663 | 83817 | 19:6962233 |
| 83826 | 19:7152680 | 83862 | 19:7718388 | 83876 | 19:8025749 | 83881 | 19:8096180 | 83923 | 19:8635132 |
| 84010 | 19:10293568 | 84014 | 19:10327116 | 84026 | 19:10599040 | 84071 | 19:11761802 | 84072 | 19:11769174 |
| 84089 | 19:12304263 | 84170 | 19:13636619 | 84217 | 19:14667736 | 84289 | 19:16236117 | 84327 | 19:16897668 |
| 84339 | 19:17077928 | 84357 | 19:17402114 | 84369 | 19:17494959 | 84377 | 19:17756500 | 84400 | 19:18264868 |
| 84459 | 19:19573243 | 84465 | 19:19674513 | 84603 | 19:21820203 | 84607 | 19:21992770 | 84702 | 19:23904908 |
| 84739 | 19:24347524 | 84755 | 19:24595008 | 84785 | 19:24888212 | 84819 | 19:25338790 | 84846 | 19:25706233 |
| 84863 | 19:26003355 | 85007 | 19:28109982 | 85052 | 19:29119243 | 85053 | 19:29121068 | 85078 | 19:29541741 |
| 85111 | 19:30203261 | 85117 | 19:30384170 | 85124 | 19:30490687 | 85126 | 19:30515659 | 85144 | 19:30924489 |
| 85204 | 19:32369220 | 85387 | 19:35695286 | 85431 | 19:36295776 | 85435 | 19:36339138 | 85474 | 19:36999184 |
| 85492 | 19:37433519 | 85531 | 19:38678725 | 85532 | 19:38692719 | 85628 | 19:40886898 | 85633 | 19:40929680 |
| 85663 | 19:41630709 | 85664 | 19:41637084 | 85678 | 19:41804462 | 85753 | 19:43355438 | 85773 | 19:43531840 |
| 85794 | 19:44097163 | 85831 | 19:44865863 | 85905 | 19:46015589 | 85937 | 19:47051179 | 85964 | 19:47510334 |
| 85968 | 19:47580297 | 85999 | 19:48115274 | 86024 | 19:48601037 | 86035 | 19:48902647 | 86036 | 19:48931565 |
| 86071 | 19:49607062 | 86084 | 19:49887742 | 86208 | 19:51505417 | 86224 | 19:51685255 | 86249 | 19:52343433 |
| 86364 | 19:54493744 | 86401 | 19:55196487 | 86568 | 19:56726108 | 86587 | 19:57128485 | 86617 | 19:57698375 |
| 86625 | 19:58000641 | 86641 | 19:58231133 | 86813 | 19:61050416 | 86922 | 19:62757680 | 87001 | 20:973990 |
| 87030 | 20:1623518 | 87122 | 20:3090504 | 87167 | 20:4139957 | 87275 | 20:4685771 | 87328 | 20:4885163 |
| 87329 | 20:4922714 | 87330 | 20:4927232 | 87333 | 20:4936772 | 87428 | 20:5478088 | 87551 | 20:7762309 |
| 87571 | 20:8209150 | 87587 | 20:8561631 | 87601 | 20:8997165 | 87640 | 20:9653842 | 87783 | 20:12796928 |
| 87828 | 20:14014441 | 87898 | 20:16397316 | 87943 | 20:17594397 | 88015 | 20:19691235 | 88233 | 20:23492042 |
| 88295 | 20:24462304 | 88299 | 20:24489532 | 88400 | 20:27494213 | 88428 | 20:28090982 | 88464 | 20:28752172 |
| 88487 | 20:29353765 | 88516 | 20:30159496 | 88606 | 20:32897468 | 88712 | 20:35378617 | 88730 | 20:35746614 |
| 88754 | 20:36168903 | 88765 | 20:36410567 | 88793 | 20:37052070 | 88823 | 20:37587442 | 88896 | 20:39414225 |
| 88910 | 20:39546261 | 88982 | 20:41144615 | 89021 | 20:41891717 | 89030 | 20:42067151 | 89088 | 20:43711039 |
| 89200 | 20:46860182 | 89213 | 20:47186127 | 89219 | 20:47416742 | 89252 | 20:48508646 | 89386 | 20:52743029 |
| 89390 | 20:52943893 | 89391 | 20:52951946 | 89435 | 20:54078693 | 89459 | 20:54797651 | 89501 | 20:55920256 |
| 89547 | 20:56907438 | 89580 | 20:57704990 | 89603 | 20:58030736 | 89614 | 20:58246871 | 89619 | 20:58356797 |
| 89640 | 20:58731368 | 89642 | 20:58744546 | 89643 | 20:58745645 | 89644 | 20:58746207 | 89645 | 20:58747818 |
| 89646 | 20:58748682 | 89658 | 20:58932980 | 89659 | 20:58935094 | 89663 | 20:59004178 | 89666 | 20:59061779 |
| 89667 | 20:59062045 | 89674 | 20:59260188 | 89684 | 20:59558955 | 89695 | 20:59880963 | 89706 | 20:60058642 |
| 89712 | 20:60329519 | 89766 | 20:61949465 | 89781 | 20:62256568 | 89802 | 20:62677110 | 89850 | 20:63825165 |
| 89883 | 20:64360760 | 89988 | 20:66882747 | 89994 | 20:67014371 | 90009 | 20:67434867 | 90052 | 20:68158435 |
| 90064 | 20:68358778 | 90067 | 20:68381160 | 90101 | 20:68986868 | 90122 | 20:69449647 | 90149 | 20:70017030 |
| 90171 | 20:70615282 | 90299 | 21:1225541 | 90386 | 21:4129789 | 90390 | 21:4195328 | 90416 | 21:5081944 |
| 90422 | 21:5168513 | 90434 | 21:5505304 | 90441 | 21:5776117 | 90445 | 21:5799099 | 90449 | 21:5865604 |
| 90466 | 21:6422404 | 90500 | 21:7408212 | 90507 | 21:7585642 | 90558 | 21:8258222 | 90607 | 21:9489941 |
| 90625 | 21:9809810 | 90673 | 21:10967078 | 90681 | 21:11128214 | 90691 | 21:11412794 | 90733 | 21:12480366 |
| 90757 | 21:12890512 | 90783 | 21:13556460 | 90795 | 21:13850729 | 90814 | 21:14283158 | 90820 | 21:14450483 |
| 90838 | 21:14883665 | 90844 | 21:15011676 | 90955 | 21:17435585 | 90977 | 21:17918237 | 90983 | 21:18074742 |
| 91028 | 21:18934542 | 91038 | 21:19207258 | 91040 | 21:19228388 | 91042 | 21:19270043 | 91051 | 21:19367037 |
| 91086 | 21:19786899 | 91094 | 21:19865924 | 91121 | 21:20591184 | 91130 | 21:20646358 | 91220 | 21:22407987 |
| 91241 | 21:22944882 | 91282 | 21:24017992 | 91292 | 21:24359607 | 91349 | 21:26122546 | 91359 | 21:26345324 |
| 91365 | 21:26551273 | 91366 | 21:26568774 | 91378 | 21:26770078 | 91419 | 21:28090529 | 91451 | 21:29134066 |
| 91457 | 21:29367658 | 91458 | 21:29377639 | 91460 | 21:29411882 | 91490 | 21:30232035 | 91576 | 21:32380295 |
| 91580 | 21:32474563 | 91616 | 21:33229274 | 91662 | 21:33796466 | 91708 | 21:34496870 | 91781 | 21:35885347 |
| 91785 | 21:35973488 | 91802 | 21:36413785 | 91865 | 21:38466535 | 91868 | 21:38504846 | 91983 | 21:41529939 |
| 91990 | 21:41713492 | 92010 | 21:42131229 | 92100 | 21:44218636 | 92101 | 21:44266061 | 92104 | 21:44392496 |
| 92121 | 21:44681616 | 92122 | 21:44686161 | 92126 | 21:44715443 | 92130 | 21:44791184 | 92173 | 21:46307247 |
| 92174 | 21:46319841 | 92198 | 21:46784418 | 92299 | 21:48659647 | 92354 | 21:49876133 | 92365 | 21:50214540 |
| 92408 | 21:51612911 | 92464 | 21:53191938 | 92484 | 21:53761679 | 92513 | 21:54536881 | 92561 | 21:55372174 |
| 92563 | 21:55394291 | 92584 | 21:55724213 | 92598 | 21:56016789 | 92610 | 21:56129095 | 92628 | 21:56595068 |
| 92629 | 21:56609576 | 92630 | 21:56612935 | 92631 | 21:56613938 | 92632 | 21:56629591 | 92638 | 21:56817682 |
| 92639 | 21:56827233 | 92640 | 21:56835873 | 92641 | 21:56855414 | 92725 | 21:57245956 | 92790 | 21:58808829 |
| 92799 | 21:59077402 | 92811 | 21:59261689 | 92842 | 21:59841484 | 92856 | 21:59994711 | 92944 | 21:61845132 |
| 92947 | 21:61911883 | 92965 | 21:62176120 | 93011 | 21:63503056 | 93031 | 21:63983849 | 93033 | 21:64015058 |
| 93060 | 21:64546209 | 93125 | 21:65454701 | 93128 | 21:65507947 | 93165 | 21:66297320 | 93293 | 21:68995998 |
| 93474 | 22:3262622 | 93623 | 22:6202218 | 93632 | 22:6324139 | 93633 | 22:6332422 | 93693 | 22:7609231 |
| 93729 | 22:8491450 | 93766 | 22:9678468 | 93843 | 22:11157683 | 93862 | 22:11729652 | 93938 | 22:13149187 |
| 93946 | 22:13475890 | 94015 | 22:15394645 | 94026 | 22:15571331 | 94029 | 22:15621273 | 94030 | 22:15622509 |
| 94033 | 22:15666979 | 94043 | 22:15998599 | 94094 | 22:17137009 | 94106 | 22:17528025 | 94190 | 22:19523346 |
| 94236 | 22:20865658 | 94239 | 22:20892499 | 94240 | 22:20896013 | 94242 | 22:20900134 | 94248 | 22:21007069 |
| 94251 | 22:21064016 | 94261 | 22:21320700 | 94267 | 22:21445580 | 94279 | 22:21651340 | 94323 | 22:22860295 |
| 94327 | 22:22968191 | 94361 | 22:23904619 | 94386 | 22:24826985 | 94425 | 22:25962309 | 94516 | 22:28260377 |
| 94558 | 22:29032150 | 94569 | 22:29394147 | 94574 | 22:29509152 | 94575 | 22:29521016 | 94599 | 22:30171429 |
| 94614 | 22:30572708 | 94689 | 22:32709942 | 94699 | 22:32992978 | 94735 | 22:34025325 | 94738 | 22:34062706 |
| 94762 | 22:34833811 | 94770 | 22:34980123 | 94821 | 22:35930996 | 94832 | 22:36212273 | 94838 | 22:36427709 |
| 94897 | 22:37678461 | 94930 | 22:38553325 | 94957 | 22:39166570 | 94961 | 22:39195248 | 94982 | 22:39426381 |
| 94987 | 22:39550615 | 94998 | 22:39688034 | 95026 | 22:40340595 | 95073 | 22:41528657 | 95160 | 22:43561183 |
| 95169 | 22:43825654 | 95180 | 22:44046370 | 95244 | 22:45637290 | 95266 | 22:45983259 | 95343 | 22:47502907 |
| 95403 | 22:49140396 | 95406 | 22:49163015 | 95408 | 22:49233906 | 95450 | 22:50360859 | 95459 | 22:50587868 |
| 95461 | 22:50603931 | 95479 | 22:50938649 | 95519 | 22:52036264 | 95533 | 22:52343223 | 95551 | 22:52768115 |
| 95554 | 22:52841252 | 95575 | 22:53087988 | 95582 | 22:53268605 | 95650 | 22:54557160 | 95662 | 22:54685597 |
| 95702 | 22:55422111 | 95717 | 22:55662527 | 95742 | 22:55891155 | 95813 | 22:56887398 | 95816 | 22:56914900 |
| 95819 | 22:56956077 | 96032 | 23:149568 | 96113 | 23:2601798 | 96118 | 23:2653886 | 96121 | 23:2672734 |
| 96179 | 23:3852744 | 96189 | 23:3981656 | 96199 | 23:4118766 | 96251 | 23:5090449 | 96255 | 23:5153243 |
| 96280 | 23:5677678 | 96282 | 23:5716762 | 96311 | 23:6480089 | 96347 | 23:7174045 | 96353 | 23:7281266 |
| 96361 | 23:7443284 | 96593 | 23:12225865 | 96644 | 23:13147408 | 96654 | 23:13421583 | 96663 | 23:13674324 |
| 96671 | 23:13837654 | 96672 | 23:13841783 | 96674 | 23:13844745 | 96675 | 23:13850106 | 96676 | 23:13856428 |
| 96677 | 23:13860190 | 96678 | 23:13867299 | 96679 | 23:13870700 | 96680 | 23:13875916 | 96681 | 23:13885227 |
| 96708 | 23:14459570 | 96719 | 23:14657847 | 96734 | 23:14924427 | 96758 | 23:15231293 | 96802 | 23:15896256 |
| 96834 | 23:16779992 | 96854 | 23:17439199 | 96875 | 23:17872465 | 96879 | 23:17919886 | 96931 | 23:19271779 |
| 96944 | 23:19469822 | 96946 | 23:19474356 | 96988 | 23:20069380 | 97027 | 23:20827882 | 97070 | 23:21933675 |
| 97095 | 23:22572630 | 97114 | 23:23252435 | 97146 | 23:23919176 | 97176 | 23:24440913 | 97183 | 23:24589985 |
| 97237 | 23:25033379 | 97239 | 23:25047940 | 97376 | 23:27210848 | 97398 | 23:27410296 | 97713 | 23:31798120 |
| 97740 | 23:32277024 | 97758 | 23:32470121 | 97771 | 23:32757047 | 97785 | 23:33017631 | 97799 | 23:33301724 |
| 97830 | 23:34183106 | 97879 | 23:35808678 | 97919 | 23:36940417 | 97987 | 23:38818865 | 97990 | 23:38876101 |
| 98006 | 23:39270706 | 98145 | 23:40884084 | 98169 | 23:41183712 | 98196 | 23:41831694 | 98206 | 23:42044741 |
| 98238 | 23:42772846 | 98243 | 23:42890423 | 98262 | 23:43295377 | 98268 | 23:43447515 | 98302 | 23:44180408 |
| 98311 | 23:44267686 | 98338 | 23:44807132 | 98340 | 23:44830102 | 98351 | 23:45020916 | 98363 | 23:45143240 |
| 98365 | 23:45154456 | 98366 | 23:45154585 | 98367 | 23:45154695 | 98369 | 23:45170649 | 98370 | 23:45174122 |
| 98371 | 23:45194932 | 98372 | 23:45209920 | 98415 | 23:46222052 | 98487 | 23:47700808 | 98491 | 23:47790490 |
| 98613 | 23:49822048 | 98646 | 23:50347036 | 98647 | 23:50349795 | 98648 | 23:50361478 | 98695 | 23:51211166 |
| 98758 | 23:52291892 | 98780 | 24:465578 | 98795 | 24:844195 | 98914 | 24:3199425 | 99033 | 24:6106333 |
| 99089 | 24:7522593 | 99139 | 24:8885668 | 99310 | 24:11057073 | 99312 | 24:11077021 | 99344 | 24:11884545 |
| 99368 | 24:12540583 | 99376 | 24:12720792 | 99407 | 24:13477647 | 99436 | 24:14392789 | 99449 | 24:14713059 |
| 99481 | 24:15784201 | 99482 | 24:15793449 | 99520 | 24:16988712 | 99542 | 24:17540200 | 99608 | 24:19628750 |
| 99633 | 24:20394869 | 99814 | 24:23537983 | 99838 | 24:24399016 | 99917 | 24:26088773 | 99969 | 24:27563741 |
| 100022 | 24:28948556 | 100051 | 24:29641974 | 100053 | 24:29754026 | 100083 | 24:30348689 | 100125 | 24:31468489 |
| 100149 | 24:31977640 | 100204 | 24:33038149 | 100247 | 24:34170378 | 100262 | 24:34562426 | 100316 | 24:35736460 |
| 100427 | 24:39087533 | 100457 | 24:39886402 | 100506 | 24:40530911 | 100517 | 24:40707643 | 100546 | 24:41182679 |
| 100560 | 24:41739524 | 100603 | 24:42667191 | 100619 | 24:42900584 | 100623 | 24:43047337 | 100646 | 24:44068866 |
| 100667 | 24:44457109 | 100702 | 24:45155232 | 100708 | 24:45303639 | 100817 | 24:46988217 | 100842 | 24:47695031 |
| 100848 | 24:47858503 | 100861 | 24:48130487 | 100927 | 24:49987049 | 100960 | 24:50907651 | 100967 | 24:51121771 |
| 100974 | 24:51293921 | 101015 | 24:52055045 | 101040 | 24:52603746 | 101042 | 24:52612789 | 101103 | 24:54184345 |
| 101132 | 24:54726088 | 101133 | 24:54737023 | 101135 | 24:54745793 | 101174 | 24:55654866 | 101209 | 24:56364015 |
| 101214 | 24:56468941 | 101217 | 24:56488437 | 101221 | 24:56542836 | 101264 | 24:57462922 | 101329 | 24:58647028 |
| 101359 | 24:59350759 | 101409 | 24:60371452 | 101438 | 24:60938433 | 101480 | 24:61694231 | 101482 | 24:61715423 |
| 101486 | 24:61776900 | 101548 | 25:1123694 | 101554 | 25:1211629 | 101568 | 25:1368623 | 101741 | 25:2890918 |
| 101757 | 25:3291302 | 101761 | 25:3373790 | 101762 | 25:3388313 | 101779 | 25:3563948 | 101807 | 25:4175576 |
| 101833 | 25:4621429 | 101909 | 25:5758755 | 101916 | 25:5934892 | 101922 | 25:6041488 | 101970 | 25:7127243 |
| 102009 | 25:7910528 | 102017 | 25:8111580 | 102058 | 25:9294790 | 102089 | 25:9965727 | 102096 | 25:10068090 |
| 102163 | 25:11303404 | 102165 | 25:11364705 | 102181 | 25:11635729 | 102182 | 25:11657253 | 102214 | 25:12395830 |
| 102217 | 25:12420213 | 102227 | 25:12757945 | 102253 | 25:13410180 | 102261 | 25:13589337 | 102275 | 25:14025698 |
| 102289 | 25:14337887 | 102372 | 25:16006571 | 102395 | 25:16397106 | 102396 | 25:16427094 | 102524 | 25:19554407 |
| 102537 | 25:19935329 | 102562 | 25:20762754 | 102600 | 25:21483496 | 102602 | 25:21510337 | 102641 | 25:22365508 |
| 102653 | 25:22668990 | 102699 | 25:23839896 | 102714 | 25:24079936 | 102736 | 25:24542279 | 102742 | 25:24617636 |
| 102767 | 25:25119747 | 102769 | 25:25147993 | 102791 | 25:25378579 | 102816 | 25:26015769 | 102932 | 25:28026271 |
| 102975 | 25:29198572 | 102994 | 25:29627243 | 103031 | 25:30198863 | 103080 | 25:31339867 | 103118 | 25:32018635 |
| 103199 | 25:33663856 | 103229 | 25:34356818 | 103305 | 25:35625080 | 103338 | 25:36046363 | 103342 | 25:36137423 |
| 103378 | 25:36442406 | 103457 | 25:37592711 | 103460 | 25:37631841 | 103479 | 25:38076102 | 103536 | 25:39097677 |
| 103596 | 25:40251086 | 103616 | 25:40630329 | 103683 | 25:41733621 | 103706 | 26:19998 | 103766 | 26:2270743 |
| 103818 | 26:3188174 | 103819 | 26:3191375 | 103829 | 26:3361204 | 103909 | 26:5551721 | 104006 | 26:8275246 |
| 104020 | 26:8585213 | 104105 | 26:10587007 | 104135 | 26:11460715 | 104155 | 26:11751995 | 104195 | 26:13034814 |
| 104284 | 26:14982249 | 104298 | 26:15453105 | 104410 | 26:18118254 | 104413 | 26:18261377 | 104460 | 26:19187481 |
| 104469 | 26:19395441 | 104471 | 26:19411437 | 104493 | 26:19971593 | 104520 | 26:20855927 | 104538 | 26:21270336 |
| 104539 | 26:21271111 | 104540 | 26:21272246 | 104542 | 26:21276672 | 104554 | 26:21609152 | 104556 | 26:21638374 |
| 104557 | 26:21659298 | 104575 | 26:22193698 | 104576 | 26:22197941 | 104651 | 26:24350779 | 104659 | 26:24607752 |
| 104686 | 26:24921440 | 104693 | 26:25131752 | 104762 | 26:26169541 | 104768 | 26:26273875 | 104774 | 26:26426669 |
| 104788 | 26:26770718 | 104860 | 26:28504622 | 104867 | 26:28761933 | 104935 | 26:30753391 | 104952 | 26:31231421 |
| 104953 | 26:31240168 | 105002 | 26:32486347 | 105010 | 26:32645140 | 105037 | 26:33152552 | 105049 | 26:33514228 |
| 105055 | 26:33591046 | 105060 | 26:33741604 | 105081 | 26:34086479 | 105174 | 26:36209430 | 105228 | 26:37411119 |
| 105255 | 26:38327739 | 105259 | 26:38422867 | 105266 | 26:38582767 | 105287 | 26:39039410 | 105319 | 26:39645163 |
| 105321 | 26:39706475 | 105436 | 26:42682884 | 105480 | 26:43924743 | 105484 | 26:44008043 | 105557 | 26:45531818 |
| 105583 | 26:46019051 | 105611 | 26:46544547 | 105633 | 26:47031869 | 105741 | 26:49412031 | 105763 | 26:49860726 |
| 105823 | 26:51197832 | 105827 | 26:51273644 | 105828 | 26:51339616 | 105832 | 26:51408594 | 105941 | 27:2266510 |
| 105949 | 27:2424175 | 106008 | 27:3926602 | 106025 | 27:4205889 | 106087 | 27:5556009 | 106166 | 27:7956657 |
| 106176 | 27:8174752 | 106200 | 27:8748506 | 106216 | 27:9098489 | 106217 | 27:9098570 | 106218 | 27:9098663 |
| 106219 | 27:9099087 | 106220 | 27:9099279 | 106221 | 27:9099292 | 106222 | 27:9099380 | 106223 | 27:9099465 |
| 106224 | 27:9099696 | 106225 | 27:9099832 | 106226 | 27:9104046 | 106228 | 27:9106497 | 106230 | 27:9110724 |
| 106231 | 27:9116774 | 106232 | 27:9117417 | 106233 | 27:9117873 | 106234 | 27:9117874 | 106235 | 27:9118114 |
| 106236 | 27:9118136 | 106237 | 27:9118300 | 106240 | 27:9149222 | 106241 | 27:9151257 | 106256 | 27:9578023 |
| 106335 | 27:12004591 | 106383 | 27:13309317 | 106461 | 27:14313738 | 106474 | 27:14529862 | 106519 | 27:15481603 |
| 106554 | 27:16106255 | 106656 | 27:18041339 | 106668 | 27:18444933 | 106677 | 27:18618418 | 106687 | 27:18831735 |
| 106688 | 27:18844548 | 106691 | 27:18895158 | 106692 | 27:18916416 | 106693 | 27:18921032 | 106756 | 27:20229425 |
| 106790 | 27:21326986 | 106801 | 27:21699196 | 106805 | 27:21744178 | 106817 | 27:21948844 | 106848 | 27:22823774 |
| 106884 | 27:23610622 | 106908 | 27:24137880 | 107014 | 27:26432887 | 107082 | 27:27644914 | 107095 | 27:27964245 |
| 107202 | 27:30418455 | 107334 | 27:32927582 | 107343 | 27:33025196 | 107408 | 27:34899643 | 107448 | 27:35233200 |
| 107456 | 27:35414868 | 107653 | 27:39221884 | 107712 | 27:40883338 | 107746 | 27:41747888 | 107759 | 27:42126889 |
| 107801 | 27:43079592 | 107803 | 27:43088850 | 107843 | 27:43939855 | 107846 | 27:43979622 | 107847 | 27:43983990 |
| 107854 | 27:44107624 | 107862 | 27:44229578 | 107939 | 28:1012326 | 107958 | 28:1304486 | 107974 | 28:1564870 |
| 108000 | 28:2124177 | 108040 | 28:3650698 | 108041 | 28:3666554 | 108059 | 28:4687863 | 108061 | 28:4710151 |
| 108084 | 28:5241897 | 108148 | 28:6765529 | 108153 | 28:6864730 | 108232 | 28:8673885 | 108245 | 28:9022551 |
| 108246 | 28:9025349 | 108267 | 28:9565874 | 108279 | 28:9801762 | 108289 | 28:9992581 | 108310 | 28:10520846 |
| 108311 | 28:10565818 | 108389 | 28:12680409 | 108396 | 28:12751017 | 108476 | 28:14911107 | 108581 | 28:17140257 |
| 108632 | 28:18044696 | 108660 | 28:18836541 | 108739 | 28:20933740 | 108741 | 28:20941251 | 108783 | 28:22351391 |
| 108826 | 28:23326980 | 108859 | 28:24154609 | 108866 | 28:24223951 | 108903 | 28:25048959 | 108912 | 28:25216858 |
| 109001 | 28:26844276 | 109007 | 28:27009355 | 109043 | 28:27784145 | 109052 | 28:27909599 | 109054 | 28:27910886 |
| 109073 | 28:28015394 | 109215 | 28:31286284 | 109245 | 28:32003701 | 109325 | 28:33230710 | 109348 | 28:33703563 |
| 109406 | 28:34803927 | 109413 | 28:34924460 | 109417 | 28:34965171 | 109472 | 28:36153178 | 109522 | 28:37465397 |
| 109570 | 28:38512629 | 109579 | 28:38669301 | 109622 | 28:39347343 | 109640 | 28:39934529 | 109726 | 28:40743505 |
| 109728 | 28:40802700 | 109729 | 28:40815314 | 109735 | 28:40867971 | 109741 | 28:40986070 | 109745 | 28:41069948 |
| 109774 | 28:41574166 | 109825 | 28:42451978 | 109871 | 28:43255478 | 109915 | 28:44119755 | 109971 | 28:45223304 |
| 110037 | 29:1129729 | 110066 | 29:1630821 | 110088 | 29:2215375 | 110115 | 29:2538458 | 110120 | 29:2640739 |
| 110159 | 29:3485853 | 110162 | 29:3535737 | 110211 | 29:4767448 | 110325 | 29:8136563 | 110346 | 29:8758233 |
| 110486 | 29:11888737 | 110531 | 29:12717025 | 110539 | 29:12922913 | 110611 | 29:14988640 | 110619 | 29:15140865 |
| 110686 | 29:16917842 | 110688 | 29:16949756 | 110701 | 29:17144041 | 110705 | 29:17304932 | 110731 | 29:18241209 |
| 110749 | 29:19246346 | 110842 | 29:21593668 | 110893 | 29:23085229 | 110896 | 29:23104998 | 110900 | 29:23212423 |
| 110941 | 29:24050623 | 111030 | 29:26373975 | 111053 | 29:27005732 | 111066 | 29:27532899 | 111074 | 29:27658327 |
| 111146 | 29:29021609 | 111151 | 29:29269330 | 111156 | 29:29356340 | 111204 | 29:29930474 | 111235 | 29:30673349 |
| 111259 | 29:31276001 | 111275 | 29:31521382 | 111281 | 29:31682726 | 111359 | 29:33581781 | 111361 | 29:33604607 |
| 111377 | 29:33936893 | 111379 | 29:33963898 | 111395 | 29:34390629 | 111404 | 29:34569090 | 111415 | 29:34694545 |
| 111459 | 29:35661091 | 111465 | 29:35722342 | 111509 | 29:36453498 | 111527 | 29:36872914 | 111613 | 29:39811611 |
| 111614 | 29:39822689 | 111712 | 29:41607581 | 111724 | 29:41814129 | 111745 | 29:42165191 | 111770 | 29:42669048 |
| 111788 | 29:43153261 | 111839 | 29:43881785 | 111856 | 29:44096512 | 111873 | 29:44366521 | 111948 | 29:46176719 |
| 111954 | 29:46281096 | 112022 | 29:47700163 | 112024 | 29:47731596 | 112148 | 29:50449063 | | |

and
the sites of the fifth marker panel are shown in the following table:

| NO. | Position | NO. | Position | NO. | Position | NO. | Position | NO. | Position |
|---|---|---|---|---|---|---|---|---|---|
| 36 | 1:1247889 | 37 | 1:1248205 | 38 | 1:1248699 | 39 | 1:1252437 | 46 | 1:1417011 |
| 47 | 1:1438871 | 48 | 1:1439812 | 51 | 1:1473953 | 52 | 1:1474233 | 53 | 1:1505639 |
| 54 | 1:1517594 | 55 | 1:1518088 | 56 | 1:1523007 | 57 | 1:1524793 | 58 | 1:1540344 |
| 59 | 1:1540422 | 60 | 1:1540458 | 61 | 1:1540555 | 119 | 1:2769164 | 139 | 1:3245141 |
| 144 | 1:3303269 | 169 | 1:3679697 | 170 | 1:3679721 | 171 | 1:3687775 | 185 | 1:3908027 |
| 207 | 1:4250046 | 209 | 1:4276065 | 400 | 1:8645661 | 446 | 1:9625272 | 607 | 1:13657759 |
| 715 | 1:16486738 | 725 | 1:16754495 | 738 | 1:17263605 | 750 | 1:17537664 | 784 | 1:18789066 |
| 935 | 1:23001427 | 965 | 1:23946108 | 991 | 1:24758865 | 993 | 1:24782983 | 1041 | 1:26105452 |
| 1093 | 1:27491647 | 1117 | 1:28566525 | 1118 | 1:28612680 | 1152 | 1:29520198 | 1205 | 1:31082779 |
| 1284 | 1:33805826 | 1294 | 1:33964232 | 1471 | 1:38630217 | 1603 | 1:42156367 | 1607 | 1:42205645 |
| 1646 | 1:43258942 | 1650 | 1:43390633 | 1777 | 1:46016435 | 1879 | 1:49422581 | 1953 | 1:51785197 |
| 1964 | 1:52229363 | 1967 | 1:52281229 | 2171 | 1:57147119 | 2172 | 1:57180246 | 2296 | 1:60247154 |
| 2297 | 1:60275851 | 2332 | 1:61100002 | 2339 | 1:61219155 | 2362 | 1:61796684 | 2462 | 1:64193131 |
| 2469 | 1:64272382 | 2530 | 1:65583709 | 2532 | 1:65625274 | 2535 | 1:65763047 | 2669 | 1:69022641 |
| 2694 | 1:69479795 | 2715 | 1:69942383 | 2774 | 1:70823693 | 2916 | 1:74460838 | 3081 | 1:78554220 |
| 3121 | 1:79513248 | 3351 | 1:85398326 | 3352 | 1:85456698 | 3354 | 1:85501954 | 3382 | 1:86229426 |
| 3387 | 1:86366405 | 3411 | 1:87047388 | 3436 | 1:87762016 | 3496 | 1:89623272 | 3548 | 1:90545947 |
| 3594 | 1:91706588 | 3654 | 1:93372952 | 3656 | 1:93477242 | 3826 | 1:97778617 | 3827 | 1:97802384 |
| 3832 | 1:97878133 | 3860 | 1:98721777 | 3895 | 1:99586706 | 4190 | 1:108556096 | 4194 | 1:108577099 |
| 4350 | 1:112307236 | 4383 | 1:112837296 | 4428 | 1:113860732 | 4434 | 1:114102926 | 4466 | 1:114828912 |
| 4467 | 1:114837165 | 4530 | 1:116786787 | 4560 | 1:117406892 | 4601 | 1:118129247 | 4619 | 1:118286166 |
| 4638 | 1:118603687 | 4640 | 1:118638525 | 4644 | 1:118785100 | 4645 | 1:118808555 | 4676 | 1:119387495 |
| 4706 | 1:120231880 | 4757 | 1:121536001 | 4852 | 1:124087395 | 4901 | 1:125202968 | 4921 | 1:125609019 |
| 4991 | 1:127321191 | 5033 | 1:128301452 | 5034 | 1:128336208 | 5132 | 1:130745923 | 5143 | 1:131052225 |
| 5212 | 1:131617330 | 5252 | 1:132852921 | 5255 | 1:132924609 | 5257 | 1:132963363 | 5326 | 1:134972795 |
| 5386 | 1:135785234 | 5387 | 1:135785949 | 5388 | 1:135787843 | 5389 | 1:135788916 | 5390 | 1:135789433 |
| 5391 | 1:135791112 | 5448 | 1:137501805 | 5548 | 1:140642688 | 5607 | 1:141638716 | 5670 | 1:142753326 |
| 5736 | 1:143879207 | 5833 | 1:145815334 | 5857 | 1:146000862 | 5863 | 1:146077457 | 5868 | 1:146242466 |
| 5876 | 1:146437585 | 5882 | 1:146612774 | 5891 | 1:146975346 | 5965 | 1:148149815 | 6115 | 1:150888684 |
| 6274 | 1:153150228 | 6320 | 1:153989230 | 6340 | 1:154314444 | 6342 | 1:154371107 | 6343 | 1:154385540 |
| 6547 | 2:682101 | 6574 | 2:1161894 | 6626 | 2:2113211 | 6636 | 2:2405667 | 6699 | 2:4200142 |
| 6781 | 2:5668859 | 6810 | 2:6248437 | 6811 | 2:6253061 | 6813 | 2:6269804 | 6815 | 2:6275413 |
| 6834 | 2:6717203 | 6835 | 2:6717548 | 6836 | 2:6732409 | 6837 | 2:6732955 | 6873 | 2:7379901 |
| 6888 | 2:7706163 | 6911 | 2:8334857 | 6932 | 2:8969249 | 7019 | 2:11681348 | 7024 | 2:11775775 |
| 7049 | 2:12802862 | 7070 | 2:13346155 | 7119 | 2:14632486 | 7120 | 2:14676143 | 7125 | 2:14743753 |
| 7148 | 2:15330545 | 7152 | 2:15479415 | 7159 | 2:15620578 | 7175 | 2:15995516 | 7188 | 2:16261766 |
| 7244 | 2:17777834 | 7245 | 2:17826748 | 7280 | 2:18272965 | 7294 | 2:18380242 | 7300 | 2:18552906 |
| 7374 | 2:20073035 | 7420 | 2:21391926 | 7484 | 2:23026213 | 7579 | 2:24732559 | 7750 | 2:28419342 |
| 7751 | 2:28419366 | 7752 | 2:28421072 | 8028 | 2:35888381 | 8230 | 2:41375205 | 8293 | 2:42660415 |
| 8319 | 2:43375192 | 8449 | 2:46777670 | 8450 | 2:46782605 | 8452 | 2:46797429 | 8466 | 2:47070734 |
| 8467 | 2:47100164 | 8549 | 2:49214428 | 8560 | 2:49760042 | 8561 | 2:49760735 | 8735 | 2:54491736 |
| 8791 | 2:55960682 | 8792 | 2:55979085 | 9079 | 2:63568686 | 9094 | 2:64064953 | 9161 | 2:65602756 |
| 9288 | 2:69830690 | 9347 | 2:71285767 | 9373 | 2:72193932 | 9374 | 2:72216636 | 9516 | 2:76002159 |
| 9530 | 2:76376460 | 9555 | 2:76976290 | 9786 | 2:83877880 | 9791 | 2:84089700 | 10121 | 2:91931085 |
| 10139 | 2:92377009 | 10180 | 2:93321855 | 10182 | 2:93326125 | 10183 | 2:93326273 | 10184 | 2:93327304 |
| 10185 | 2:93353835 | 10186 | 2:93353877 | 10187 | 2:93354446 | 10188 | 2:93354619 | 10189 | 2:93354804 |
| 10190 | 2:93355262 | 10191 | 2:93355271 | 10192 | 2:93356138 | 10193 | 2:93356208 | 10194 | 2:93356223 |
| 10195 | 2:93366311 | 10248 | 2:94636270 | 10286 | 2:95449694 | 10305 | 2:95979209 | 10312 | 2:96139223 |
| 10401 | 2:98337087 | 10409 | 2:98421675 | 10413 | 2:98461459 | 10417 | 2:98536760 | 10466 | 2:99940359 |
| 10605 | 2:103776260 | 10655 | 2:105009833 | 10686 | 2:105635824 | 10694 | 2:105828123 | 10712 | 2:106230731 |
| 10781 | 2:107728454 | 10782 | 2:107728517 | 10783 | 2:107729520 | 10784 | 2:107730636 | 10785 | 2:107735095 |
| 10786 | 2:107735449 | 10787 | 2:107737533 | 10789 | 2:107742839 | 10800 | 2:107971542 | 10846 | 2:109019023 |
| 10929 | 2:111354781 | 10934 | 2:111635526 | 10954 | 2:112098945 | 10956 | 2:112105210 | 10975 | 2:112632558 |
| 10978 | 2:112654694 | 11038 | 2:113914830 | 11099 | 2:115533946 | 11303 | 2:120430113 | 11405 | 2:123285701 |
| 11444 | 2:124279927 | 11460 | 2:124841893 | 11684 | 2:128718214 | 11813 | 2:131557054 | 11822 | 2:131862099 |
| 11852 | 2:132351992 | 11940 | 2:133852858 | 11943 | 2:133878448 | 11959 | 2:134115936 | 11960 | 2:134172533 |
| 12004 | 2:134750704 | 12059 | 2:135525484 | 12143 | 3:950659 | 12145 | 3:954886 | 12187 | 3:1728119 |
| 12274 | 3:3596795 | 12343 | 3:4727841 | 12452 | 3:6832814 | 12454 | 3:6841596 | 12456 | 3:6848201 |
| 12457 | 3:6866175 | 12458 | 3:6867557 | 12459 | 3:6867867 | 12460 | 3:6885163 | 12533 | 3:8025174 |
| 12734 | 3:11445314 | 12790 | 3:12934994 | 12856 | 3:14226434 | 12920 | 3:15616501 | 12948 | 3:15994930 |
| 13048 | 3:18771436 | 13049 | 3:18775018 | 13138 | 3:21163145 | 13156 | 3:21764453 | 13190 | 3:22332383 |
| 13191 | 3:22334033 | 13192 | 3:22339164 | 13193 | 3:22340631 | 13194 | 3:22343298 | 13357 | 3:26160439 |
| 13470 | 3:27968845 | 13475 | 3:28047336 | 13527 | 3:29246450 | 13584 | 3:30235042 | 13600 | 3:30605190 |
| 13623 | 3:31149250 | 13660 | 3:31948925 | 13662 | 3:31976577 | 13736 | 3:32970665 | 13765 | 3:33258971 |
| 14049 | 3:40032182 | 14051 | 3:40065534 | 14053 | 3:40120645 | 14188 | 3:43541601 | 14331 | 3:47791771 |
| 14354 | 3:48191758 | 14355 | 3:48234094 | 14392 | 3:49000989 | 14416 | 3:49459128 | 14422 | 3:49526251 |
| 14428 | 3:49574985 | 14483 | 3:49988687 | 14504 | 3:50656944 | 14581 | 3:52783346 | 14661 | 3:55239885 |
| 14665 | 3:55330131 | 14835 | 3:60375726 | 14845 | 3:60575455 | 14850 | 3:60636353 | 14868 | 3:60938213 |
| 14958 | 3:63728694 | 14960 | 3:63776877 | 14965 | 3:63861865 | 14966 | 3:63887795 | 14968 | 3:63950495 |
| 14969 | 3:63955495 | 14970 | 3:63976119 | 14973 | 3:64020621 | 14975 | 3:64058308 | 14983 | 3:64239387 |
| 14988 | 3:64525583 | 14992 | 3:64697961 | 15033 | 3:65670943 | 15044 | 3:66042478 | 15049 | 3:66148244 |
| 15107 | 3:67465318 | 15130 | 3:67947626 | 15131 | 3:67978806 | 15150 | 3:68443615 | 15175 | 3:69034450 |
| 15230 | 3:70652398 | 15234 | 3:70860935 | 15243 | 3:71230722 | 15285 | 3:72930619 | 15393 | 3:76153357 |
| 15442 | 3:77599781 | 15443 | 3:77635657 | 15446 | 3:77784239 | 15495 | 3:79128870 | 15498 | 3:79224313 |
| 15538 | 3:80529583 | 15563 | 3:81048556 | 15605 | 3:82166845 | 15625 | 3:82581418 | 15684 | 3:84280062 |
| 15939 | 3:89312804 | 15941 | 3:89315922 | 16034 | 3:91258808 | 16253 | 3:96974610 | 16259 | 3:97049079 |
| 16287 | 3:97630051 | 16414 | 3:100563623 | 16430 | 3:100751713 | 16431 | 3:100782663 | 16443 | 3:101112794 |
| 16446 | 3:101150016 | 16537 | 3:102883747 | 16541 | 3:102931082 | 16598 | 3:104333611 | 16602 | 3:104484207 |
| 16605 | 3:104506752 | 16607 | 3:104541890 | 16609 | 3:104556518 | 16628 | 3:104869949 | 16646 | 3:105209686 |
| 16654 | 3:105292781 | 16714 | 3:105965567 | 16715 | 3:105973059 | 16718 | 3:106026621 | 16719 | 3:106042336 |
| 16740 | 3:106492826 | 16750 | 3:106599058 | 16901 | 3:109689309 | 17056 | 3:113011961 | 17068 | 3:113301217 |
| 17115 | 3:113990093 | 17186 | 3:114897383 | 17380 | 3:117882488 | 17384 | 3:117909372 | 17401 | 3:118223118 |
| 17402 | 3:118253078 | 17639 | 4:4003154 | 17644 | 4:4149740 | 17697 | 4:5004514 | 17705 | 4:5165078 |
| 17706 | 4:5173632 | 17740 | 4:5747787 | 17752 | 4:6130688 | 17761 | 4:6330228 | 17968 | 4:10578187 |
| 17969 | 4:10580814 | 18198 | 4:16670558 | 18273 | 4:18595000 | 18276 | 4:18619515 | 18318 | 4:19200647 |
| 18378 | 4:20303118 | 18635 | 4:26901086 | 18636 | 4:26934291 | 18820 | 4:32010653 | 18843 | 4:32768824 |
| 18949 | 4:35971843 | 19100 | 4:40158341 | 19134 | 4:40946237 | 19168 | 4:41815635 | 19173 | 4:42048079 |
| 19208 | 4:43120197 | 19214 | 4:43203876 | 19250 | 4:44121669 | 19285 | 4:45161683 | 19356 | 4:47190294 |
| 19377 | 4:47463756 | 19400 | 4:48100380 | 19424 | 4:48952233 | 19466 | 4:49461342 | 19477 | 4:49813182 |
| 19478 | 4:49842196 | 19480 | 4:49888592 | 19866 | 4:61781138 | 19888 | 4:62276469 | 19891 | 4:62357256 |
| 19931 | 4:63512286 | 19965 | 4:64527362 | 19982 | 4:64979970 | 20028 | 4:66103436 | 20075 | 4:67374218 |
| 20136 | 4:69236841 | 20289 | 4:73424213 | 20513 | 4:78984643 | 20553 | 4:80105286 | 20554 | 4:80107022 |
| 20555 | 4:80107183 | 20556 | 4:80107305 | 20557 | 4:80107320 | 20558 | 4:80107386 | 20559 | 4:80107434 |
| 20560 | 4:80107694 | 20561 | 4:80107717 | 20562 | 4:80107759 | 20674 | 4:82773442 | 20681 | 4:82863329 |
| 20749 | 4:85034221 | 20779 | 4:85832979 | 20780 | 4:85866052 | 20788 | 4:85968886 | 20834 | 4:87033808 |
| 20882 | 4:88417629 | 20886 | 4:88515168 | 20903 | 4:89049127 | 21046 | 4:92398977 | 21050 | 4:92448883 |
| 21051 | 4:92451008 | 21052 | 4:92451165 | 21109 | 4:93517701 | 21117 | 4:93652019 | 21185 | 4:95523178 |
| 21553 | 4:100762424 | 21603 | 4:101689426 | 21683 | 4:103739455 | 21711 | 4:104521615 | 21718 | 4:104699947 |
| 21818 | 4:106683233 | 21824 | 4:106777370 | 21836 | 4:106986917 | 21886 | 4:107625627 | 21968 | 4:109840544 |
| 22030 | 4:111174187 | 22078 | 4:111934020 | 22116 | 4:112499124 | 22123 | 4:112606237 | 22134 | 4:112692746 |
| 22137 | 4:112719377 | 22140 | 4:112741810 | 22149 | 4:112797073 | 22180 | 4:113503432 | 22250 | 4:114401519 |
| 22253 | 4:114443077 | 22274 | 4:114788184 | 22278 | 4:114812550 | 22312 | 4:115192347 | 22318 | 4:115316340 |
| 22319 | 4:115317642 | 22354 | 4:116067653 | 22376 | 4:116420553 | 22394 | 4:116749463 | 22406 | 4:116987390 |
| 22513 | 4:119160825 | 22525 | 4:119536985 | 22581 | 5:882265 | 22670 | 5:2514974 | 22693 | 5:3086065 |
| 22707 | 5:3684231 | 22731 | 5:4371208 | 22740 | 5:4517412 | 22761 | 5:5025515 | 22776 | 5:5256216 |
| 22899 | 5:8017094 | 22935 | 5:8963886 | 22937 | 5:9058312 | 22939 | 5:9081371 | 22941 | 5:9110521 |
| 22974 | 5:9917938 | 23184 | 5:14901254 | 23203 | 5:15246344 | 23335 | 5:18879061 | 23340 | 5:18957429 |
| 23448 | 5:21803211 | 23511 | 5:22947860 | 23513 | 5:23010158 | 23563 | 5:23986445 | 23619 | 5:25424207 |
| 23628 | 5:25663032 | 23683 | 5:27040938 | 23685 | 5:27062257 | 23767 | 5:28682732 | 23795 | 5:29481439 |
| 23894 | 5:31935957 | 23904 | 5:32259633 | 23908 | 5:32334842 | 23922 | 5:32649736 | 23951 | 5:33311326 |
| 24015 | 5:35004932 | 24046 | 5:36022885 | 24051 | 5:36113810 | 24053 | 5:36193854 | 24092 | 5:37455345 |
| 24105 | 5:37749836 | 24243 | 5:41293748 | 24244 | 5:41337074 | 24247 | 5:41451300 | 24254 | 5:41682610 |
| 24265 | 5:41958597 | 24268 | 5:42010622 | 24275 | 5:42382038 | 24295 | 5:42883310 | 24301 | 5:43024524 |
| 24312 | 5:43371061 | 24352 | 5:44118721 | 24387 | 5:45222817 | 24450 | 5:46743902 | 24683 | 5:52698528 |
| 24690 | 5:53009069 | 25020 | 5:61964739 | 25024 | 5:62044690 | 25041 | 5:62542053 | 25049 | 5:62915639 |
| 25350 | 5:69917738 | 25398 | 5:71197728 | 25467 | 5:72417962 | 25469 | 5:72465277 | 25527 | 5:74079648 |
| 25639 | 5:76766433 | 25642 | 5:76818912 | 25644 | 5:76890153 | 25645 | 5:76972053 | 25757 | 5:80114008 |
| 25760 | 5:80235852 | 25784 | 5:80982109 | 25787 | 5:81116016 | 25814 | 5:81935764 | 25875 | 5:83029733 |
| 26074 | 5:84527341 | 26206 | 5:87607418 | 26217 | 5:87838676 | 26304 | 5:89147995 | 26364 | 5:90614584 |
| 26395 | 5:91363881 | 26508 | 5:93490294 | 26558 | 5:94637080 | 26603 | 5:95645793 | 26605 | 5:95684692 |
| 26607 | 5:95699816 | 26610 | 5:95747447 | 26611 | 5:95748288 | 26612 | 5:95749996 | 26763 | 5:98601044 |
| 26855 | 5:100383163 | 26876 | 5:100815761 | 26879 | 5:100854950 | 26917 | 5:101625777 | 27085 | 5:105474132 |
| 27094 | 5:105732916 | 27095 | 5:105733857 | 27096 | 5:105740050 | 27097 | 5:105756724 | 27098 | 5:105760018 |
| 27099 | 5:105760750 | 27101 | 5:105770267 | 27102 | 5:105771805 | 27103 | 5:105773809 | 27104 | 5:105776080 |
| 27105 | 5:105776475 | 27106 | 5:105778521 | 27107 | 5:105778777 | 27108 | 5:105779368 | 27109 | 5:105780258 |
| 27110 | 5:105800847 | 27112 | 5:105804880 | 27167 | 5:106540440 | 27175 | 5:106650676 | 27227 | 5:107423031 |
| 27378 | 5:110192579 | 27381 | 5:110289240 | 27391 | 5:110386083 | 27419 | 5:110751512 | 27501 | 5:112519431 |
| 27502 | 5:112553713 | 27533 | 5:113205459 | 27568 | 5:114190671 | 27822 | 5:117930669 | 27862 | 5:118720245 |
| 27891 | 5:119036022 | 27910 | 5:119441240 | 28029 | 6:1993838 | 28117 | 6:3589994 | 28156 | 6:4424160 |
| 28249 | 6:6458237 | 28250 | 6:6482619 | 28394 | 6:10017733 | 28447 | 6:11463553 | 28470 | 6:11875946 |
| 28484 | 6:12191468 | 28503 | 6:12548297 | 28504 | 6:12550413 | 28512 | 6:12753537 | 28513 | 6:12808531 |
| 28515 | 6:12873396 | 28517 | 6:12900652 | 28580 | 6:14515552 | 28582 | 6:14546752 | 28584 | 6:14593717 |
| 28588 | 6:14737560 | 28660 | 6:16069555 | 28665 | 6:16173201 | 28670 | 6:16334539 | 28671 | 6:16387845 |
| 28672 | 6:16407876 | 28700 | 6:16918116 | 28708 | 6:17036127 | 28721 | 6:17373915 | 28727 | 6:17498903 |
| 28786 | 6:18598550 | 28800 | 6:18917943 | 28802 | 6:18955466 | 28828 | 6:19455356 | 28838 | 6:19628808 |
| 28847 | 6:19848821 | 28851 | 6:19923403 | 28858 | 6:20135992 | 28864 | 6:20273699 | 28873 | 6:20399602 |
| 28881 | 6:20600966 | 28884 | 6:20625823 | 28891 | 6:20779771 | 28892 | 6:20821807 | 28906 | 6:21288355 |
| 28909 | 6:21362764 | 28911 | 6:21407401 | 28920 | 6:21614174 | 28924 | 6:21682567 | 28929 | 6:21732184 |
| 28930 | 6:21736509 | 28937 | 6:21857115 | 28943 | 6:22080199 | 28948 | 6:22169669 | 28958 | 6:22265776 |
| 28982 | 6:22743978 | 28986 | 6:22838838 | 29009 | 6:23503327 | 29017 | 6:23665616 | 29019 | 6:23697957 |
| 29025 | 6:23885602 | 29026 | 6:23902363 | 29027 | 6:23902790 | 29037 | 6:24156443 | 29038 | 6:24188452 |
| 29062 | 6:24764063 | 29073 | 6:25006732 | 29074 | 6:25007314 | 29075 | 6:25007705 | 29085 | 6:25188129 |
| 29088 | 6:25333843 | 29095 | 6:25528726 | 29104 | 6:25843075 | 29112 | 6:25978766 | 29117 | 6:26022919 |
| 29125 | 6:26158019 | 29142 | 6:26679658 | 29146 | 6:26760274 | 29148 | 6:26792379 | 29156 | 6:27042457 |
| 29160 | 6:27154761 | 29165 | 6:27332801 | 29171 | 6:27406476 | 29192 | 6:28048764 | 29202 | 6:28285173 |
| 29219 | 6:28873303 | 29221 | 6:28936994 | 29222 | 6:28940832 | 29224 | 6:28978809 | 29229 | 6:29062262 |
| 29242 | 6:29357249 | 29246 | 6:29406846 | 29252 | 6:29478697 | 29261 | 6:29591601 | 29264 | 6:29642427 |
| 29266 | 6:29700842 | 29272 | 6:29850442 | 29275 | 6:29928530 | 29278 | 6:30059483 | 29281 | 6:30087471 |
| 29282 | 6:30092735 | 29283 | 6:30096041 | 29284 | 6:30096126 | 29285 | 6:30100327 | 29286 | 6:30101354 |
| 29288 | 6:30105186 | 29289 | 6:30107161 | 29290 | 6:30107363 | 29291 | 6:30107470 | 29292 | 6:30107503 |
| 29293 | 6:30108091 | 29294 | 6:30108190 | 29295 | 6:30108349 | 29296 | 6:30108502 | 29297 | 6:30109134 |
| 29298 | 6:30109220 | 29299 | 6:30109285 | 29300 | 6:30109297 | 29301 | 6:30109871 | 29302 | 6:30109915 |
| 29303 | 6:30110219 | 29304 | 6:30110252 | 29305 | 6:30110638 | 29306 | 6:30111065 | 29307 | 6:30111645 |
| 29308 | 6:30112226 | 29309 | 6:30112636 | 29310 | 6:30112665 | 29311 | 6:30112688 | 29312 | 6:30112989 |
| 29313 | 6:30113025 | 29314 | 6:30113099 | 29315 | 6:30113113 | 29316 | 6:30113219 | 29317 | 6:30113503 |
| 29318 | 6:30113552 | 29319 | 6:30113737 | 29320 | 6:30114877 | 29321 | 6:30114924 | 29322 | 6:30115248 |
| 29323 | 6:30115405 | 29324 | 6:30115465 | 29325 | 6:30117653 | 29326 | 6:30117880 | 29327 | 6:30117987 |
| 29328 | 6:30118079 | 29329 | 6:30118215 | 29330 | 6:30119075 | 29331 | 6:30119163 | 29332 | 6:30119239 |
| 29333 | 6:30119492 | 29334 | 6:30120062 | 29335 | 6:30120131 | 29336 | 6:30120148 | 29337 | 6:30120317 |
| 29338 | 6:30120495 | 29339 | 6:30120714 | 29340 | 6:30120745 | 29341 | 6:30121010 | 29345 | 6:30186140 |
| 29347 | 6:30231489 | 29348 | 6:30231511 | 29349 | 6:30231899 | 29350 | 6:30231913 | 29351 | 6:30232234 |
| 29356 | 6:30429744 | 29366 | 6:30575474 | 29370 | 6:30706328 | 29373 | 6:30820768 | 29381 | 6:31067757 |
| 29386 | 6:31258805 | 29390 | 6:31380855 | 29392 | 6:31425251 | 29394 | 6:31479415 | 29397 | 6:31502542 |
| 29398 | 6:31523083 | 29409 | 6:31802402 | 29417 | 6:32011040 | 29446 | 6:33106068 | 29447 | 6:33200563 |
| 29448 | 6:33249833 | 29450 | 6:33303040 | 29454 | 6:33451849 | 29456 | 6:33555068 | 29466 | 6:33983856 |
| 29474 | 6:34086214 | 29476 | 6:34139287 | 29477 | 6:34174152 | 29478 | 6:34179094 | 29479 | 6:34185107 |
| 29480 | 6:34189894 | 29481 | 6:34197024 | 29483 | 6:34206187 | 29484 | 6:34207156 | 29485 | 6:34210015 |
| 29487 | 6:34227111 | 29488 | 6:34229172 | 29489 | 6:34236680 | 29491 | 6:34238928 | 29492 | 6:34239830 |
| 29493 | 6:34248001 | 29494 | 6:34262023 | 29495 | 6:34291379 | 29496 | 6:34292850 | 29497 | 6:34293142 |
| 29498 | 6:34297896 | 29499 | 6:34298106 | 29500 | 6:34300136 | 29501 | 6:34305846 | 29502 | 6:34313535 |
| 29503 | 6:34314736 | 29504 | 6:34316483 | 29506 | 6:34317628 | 29507 | 6:34317733 | 29508 | 6:34319685 |
| 29509 | 6:34319748 | 29510 | 6:34319965 | 29511 | 6:34320099 | 29512 | 6:34320208 | 29513 | 6:34320272 |
| 29514 | 6:34320435 | 29515 | 6:34320590 | 29516 | 6:34320597 | 29517 | 6:34321475 | 29518 | 6:34321669 |
| 29519 | 6:34321700 | 29520 | 6:34322765 | 29521 | 6:34323241 | 29522 | 6:34323355 | 29523 | 6:34323382 |
| 29524 | 6:34323399 | 29525 | 6:34323857 | 29526 | 6:34323923 | 29527 | 6:34324379 | 29528 | 6:34324509 |
| 29529 | 6:34324543 | 29530 | 6:34324794 | 29531 | 6:34324813 | 29532 | 6:34324864 | 29533 | 6:34325948 |
| 29534 | 6:34326047 | 29535 | 6:34326105 | 29536 | 6:34326672 | 29537 | 6:34326907 | 29538 | 6:34326931 |
| 29539 | 6:34327343 | 29540 | 6:34328174 | 29541 | 6:34328391 | 29542 | 6:34328595 | 29543 | 6:34328871 |
| 29544 | 6:34329287 | 29545 | 6:34329583 | 29546 | 6:34329816 | 29547 | 6:34329841 | 29548 | 6:34329959 |
| 29549 | 6:34330004 | 29550 | 6:34330105 | 29551 | 6:34330219 | 29552 | 6:34330248 | 29553 | 6:34330469 |
| 29554 | 6:34330615 | 29555 | 6:34331649 | 29556 | 6:34331871 | 29557 | 6:34334654 | 29558 | 6:34334695 |
| 29559 | 6:34334819 | 29560 | 6:34334883 | 29561 | 6:34334942 | 29562 | 6:34334975 | 29563 | 6:34335273 |
| 29564 | 6:34335676 | 29565 | 6:34336082 | 29566 | 6:34336116 | 29567 | 6:34336302 | 29568 | 6:34336518 |
| 29569 | 6:34336806 | 29570 | 6:34337142 | 29571 | 6:34337235 | 29572 | 6:34337793 | 29573 | 6:34337852 |
| 29574 | 6:34337871 | 29575 | 6:34337952 | 29576 | 6:34338244 | 29577 | 6:34338458 | 29578 | 6:34338858 |
| 29579 | 6:34338912 | 29580 | 6:34341641 | 29581 | 6:34341671 | 29582 | 6:34342381 | 29583 | 6:34342418 |
| 29584 | 6:34342439 | 29585 | 6:34342876 | 29586 | 6:34343375 | 29587 | 6:34344002 | 29588 | 6:34344707 |
| 29589 | 6:34344823 | 29590 | 6:34344911 | 29591 | 6:34345770 | 29592 | 6:34345787 | 29593 | 6:34347423 |
| 29594 | 6:34347478 | 29595 | 6:34347542 | 29596 | 6:34347870 | 29597 | 6:34347912 | 29598 | 6:34348158 |
| 29599 | 6:34348200 | 29600 | 6:34350237 | 29601 | 6:34350256 | 29602 | 6:34350284 | 29603 | 6:34352029 |
| 29604 | 6:34352540 | 29605 | 6:34353080 | 29606 | 6:34353086 | 29607 | 6:34353183 | 29608 | 6:34353582 |
| 29609 | 6:34353683 | 29610 | 6:34355309 | 29611 | 6:34356066 | 29612 | 6:34356817 | 29613 | 6:34357441 |
| 29614 | 6:34357724 | 29615 | 6:34358059 | 29616 | 6:34358159 | 29617 | 6:34359885 | 29618 | 6:34359922 |
| 29619 | 6:34360320 | 29620 | 6:34360574 | 29621 | 6:34360625 | 29622 | 6:34361019 | 29623 | 6:34361442 |
| 29624 | 6:34362625 | 29625 | 6:34363701 | 29626 | 6:34366860 | 29627 | 6:34367704 | 29628 | 6:34368646 |
| 29629 | 6:34368728 | 29630 | 6:34368895 | 29631 | 6:34369801 | 29632 | 6:34370381 | 29633 | 6:34373419 |
| 29634 | 6:34375386 | 29635 | 6:34377523 | 29636 | 6:34378377 | 29637 | 6:34378764 | 29638 | 6:34379342 |
| 29639 | 6:34379765 | 29640 | 6:34380266 | 29641 | 6:34380420 | 29642 | 6:34390859 | 29643 | 6:34398273 |
| 29644 | 6:34400215 | 29645 | 6:34401918 | 29646 | 6:34405865 | 29647 | 6:34409323 | 29648 | 6:34412354 |
| 29649 | 6:34412446 | 29650 | 6:34417593 | 29651 | 6:34423276 | 29652 | 6:34425931 | 29653 | 6:34429298 |
| 29654 | 6:34433083 | 29655 | 6:34436717 | 29656 | 6:34443045 | 29657 | 6:34445489 | 29658 | 6:34454602 |
| 29661 | 6:34485191 | 29663 | 6:34508971 | 29664 | 6:34511326 | 29665 | 6:34512053 | 29666 | 6:34512149 |
| 29667 | 6:34512224 | 29668 | 6:34512436 | 29669 | 6:34512607 | 29670 | 6:34514009 | 29671 | 6:34520073 |
| 29672 | 6:34520205 | 29673 | 6:34520340 | 29674 | 6:34521287 | 29675 | 6:34521294 | 29676 | 6:34522877 |
| 29677 | 6:34523485 | 29678 | 6:34524720 | 29679 | 6:34526737 | 29680 | 6:34527170 | 29681 | 6:34531081 |
| 29682 | 6:34531219 | 29683 | 6:34531228 | 29684 | 6:34532115 | 29685 | 6:34532276 | 29686 | 6:34533970 |
| 29687 | 6:34534131 | 29688 | 6:34534507 | 29689 | 6:34534849 | 29690 | 6:34534873 | 29691 | 6:34535922 |
| 29692 | 6:34535958 | 29693 | 6:34535980 | 29694 | 6:34537513 | 29695 | 6:34538518 | 29696 | 6:34538586 |
| 29697 | 6:34538798 | 29698 | 6:34539189 | 29699 | 6:34544253 | 29700 | 6:34544296 | 29701 | 6:34544650 |
| 29702 | 6:34544882 | 29703 | 6:34544924 | 29704 | 6:34545143 | 29705 | 6:34545442 | 29706 | 6:34545976 |
| 29707 | 6:34546002 | 29708 | 6:34546098 | 29709 | 6:34546230 | 29710 | 6:34546882 | 29711 | 6:34551061 |
| 29712 | 6:34551087 | 29713 | 6:34551357 | 29714 | 6:34551821 | 29715 | 6:34552091 | 29716 | 6:34552302 |
| 29717 | 6:34559140 | 29719 | 6:34568139 | 29720 | 6:34570377 | 29721 | 6:34571403 | 29723 | 6:34585218 |
| 29724 | 6:34587383 | 29726 | 6:34590054 | 29727 | 6:34590568 | 29728 | 6:34590836 | 29729 | 6:34590891 |
| 29730 | 6:34594553 | 29731 | 6:34595913 | 29732 | 6:34605484 | 29734 | 6:34606444 | 29735 | 6:34606508 |
| 29736 | 6:34607125 | 29737 | 6:34607239 | 29738 | 6:34608122 | 29739 | 6:34608604 | 29740 | 6:34609191 |
| 29741 | 6:34609820 | 29742 | 6:34609854 | 29743 | 6:34610451 | 29744 | 6:34610729 | 29745 | 6:34610740 |
| 29746 | 6:34610765 | 29748 | 6:34610849 | 29749 | 6:34611386 | 29750 | 6:34611923 | 29751 | 6:34611955 |
| 29752 | 6:34612600 | 29753 | 6:34613447 | 29754 | 6:34614887 | 29755 | 6:34615063 | 29756 | 6:34617013 |
| 29757 | 6:34617195 | 29758 | 6:34617208 | 29759 | 6:34617293 | 29760 | 6:34617674 | 29761 | 6:34617812 |
| 29762 | 6:34617865 | 29763 | 6:34617943 | 29764 | 6:34618113 | 29765 | 6:34618226 | 29766 | 6:34618258 |
| 29767 | 6:34618461 | 29768 | 6:34618467 | 29769 | 6:34618532 | 29770 | 6:34618622 | 29771 | 6:34618679 |
| 29772 | 6:34618784 | 29773 | 6:34618813 | 29774 | 6:34618880 | 29775 | 6:34620004 | 29776 | 6:34620108 |
| 29777 | 6:34620118 | 29778 | 6:34620548 | 29779 | 6:34621825 | 29780 | 6:34622374 | 29781 | 6:34623163 |
| 29782 | 6:34623346 | 29783 | 6:34623681 | 29784 | 6:34623706 | 29785 | 6:34623745 | 29786 | 6:34624441 |
| 29787 | 6:34629181 | 29788 | 6:34629567 | 29789 | 6:34629581 | 29790 | 6:34629692 | 29791 | 6:34629900 |
| 29792 | 6:34629975 | 29793 | 6:34629990 | 29794 | 6:34630073 | 29795 | 6:34630347 | 29796 | 6:34630587 |
| 29797 | 6:34630602 | 29798 | 6:34630922 | 29799 | 6:34631246 | 29800 | 6:34631446 | 29801 | 6:34631808 |
| 29802 | 6:34631843 | 29803 | 6:34632098 | 29804 | 6:34632375 | 29805 | 6:34632405 | 29806 | 6:34632714 |
| 29807 | 6:34632833 | 29808 | 6:34633050 | 29809 | 6:34633159 | 29810 | 6:34633187 | 29811 | 6:34633229 |
| 29812 | 6:34633562 | 29813 | 6:34633782 | 29814 | 6:34633841 | 29815 | 6:34633967 | 29816 | 6:34633990 |
| 29817 | 6:34634055 | 29818 | 6:34634169 | 29819 | 6:34634190 | 29820 | 6:34634250 | 29821 | 6:34634312 |
| 29822 | 6:34634801 | 29823 | 6:34634990 | 29824 | 6:34635003 | 29825 | 6:34635530 | 29826 | 6:34635703 |
| 29827 | 6:34636131 | 29828 | 6:34636138 | 29829 | 6:34636209 | 29830 | 6:34637085 | 29831 | 6:34640553 |
| 29832 | 6:34641695 | 29833 | 6:34647407 | 29834 | 6:34657657 | 29835 | 6:34661296 | 29836 | 6:34661323 |
| 29837 | 6:34662849 | 29838 | 6:34665463 | 29839 | 6:34668000 | 29840 | 6:34670574 | 29841 | 6:34671333 |
| 29842 | 6:34673238 | 29844 | 6:34686618 | 29845 | 6:34687343 | 29846 | 6:34689725 | 29847 | 6:34697260 |
| 29848 | 6:34699079 | 29849 | 6:34703681 | 29850 | 6:34704601 | 29851 | 6:34704806 | 29852 | 6:34705455 |
| 29853 | 6:34714302 | 29854 | 6:34714956 | 29855 | 6:34724151 | 29856 | 6:34726853 | 29857 | 6:34727995 |
| 29858 | 6:34728449 | 29859 | 6:34730148 | 29860 | 6:34730862 | 29861 | 6:34734530 | 29863 | 6:34738528 |
| 29864 | 6:34746517 | 29865 | 6:34751152 | 29866 | 6:34751196 | 29867 | 6:34751889 | 29868 | 6:34753115 |
| 29869 | 6:34762474 | 29870 | 6:34762792 | 29871 | 6:34762888 | 29872 | 6:34766154 | 29873 | 6:34766590 |
| 29874 | 6:34766756 | 29875 | 6:34768280 | 29876 | 6:34768301 | 29877 | 6:34768997 | 29878 | 6:34769036 |
| 29879 | 6:34769720 | 29880 | 6:34770109 | 29881 | 6:34770808 | 29882 | 6:34777728 | 29883 | 6:34780553 |
| 29884 | 6:34781550 | 29885 | 6:34781934 | 29886 | 6:34782739 | 29887 | 6:34782759 | 29888 | 6:34783042 |
| 29889 | 6:34783961 | 29890 | 6:34784243 | 29891 | 6:34784299 | 29892 | 6:34784335 | 29893 | 6:34784977 |
| 29894 | 6:34785811 | 29895 | 6:34785864 | 29896 | 6:34785916 | 29897 | 6:34786001 | 29898 | 6:34786006 |
| 29899 | 6:34786198 | 29900 | 6:34786203 | 29901 | 6:34786474 | 29902 | 6:34786586 | 29903 | 6:34787240 |
| 29904 | 6:34789109 | 29905 | 6:34792867 | 29907 | 6:34845861 | 29909 | 6:34929550 | 29910 | 6:34932019 |
| 29911 | 6:34933373 | 29912 | 6:34943666 | 29913 | 6:34961634 | 29915 | 6:34972640 | 29916 | 6:34976990 |
| 29917 | 6:34985119 | 29918 | 6:34990679 | 29919 | 6:34992674 | 29920 | 6:34994097 | 29921 | 6:34995220 |
| 29922 | 6:35021191 | 29923 | 6:35047210 | 29924 | 6:35140608 | 29927 | 6:35157410 | 29929 | 6:35160353 |
| 29930 | 6:35161520 | 29932 | 6:35162419 | 29934 | 6:35163337 | 29935 | 6:35165014 | 29936 | 6:35166488 |
| 29937 | 6:35166725 | 29942 | 6:35245781 | 29945 | 6:35274129 | 29948 | 6:35381936 | 29953 | 6:35504728 |
| 29957 | 6:35561016 | 29958 | 6:35561546 | 29959 | 6:35561595 | 29960 | 6:35581628 | 29961 | 6:35582162 |
| 29962 | 6:35582223 | 29963 | 6:35587094 | 29964 | 6:35594486 | 29965 | 6:35609063 | 29966 | 6:35616265 |
| 29967 | 6:35616280 | 29969 | 6:35618707 | 29974 | 6:35666246 | 29975 | 6:35678704 | 29976 | 6:35678754 |
| 29977 | 6:35684254 | 29978 | 6:35703001 | 29979 | 6:35713846 | 29980 | 6:35718341 | 29981 | 6:35726574 |
| 29982 | 6:35732398 | 29984 | 6:35792391 | 29985 | 6:35825879 | 29987 | 6:35886281 | 29989 | 6:35897314 |
| 29994 | 6:35908980 | 30001 | 6:35937425 | 30002 | 6:35937819 | 30003 | 6:35937874 | 30004 | 6:35941557 |
| 30006 | 6:35948457 | 30007 | 6:35963118 | 30008 | 6:35968631 | 30009 | 6:35972831 | 30012 | 6:35992028 |
| 30014 | 6:36011027 | 30015 | 6:36011772 | 30018 | 6:36032905 | 30019 | 6:36034678 | 30020 | 6:36036595 |
| 30029 | 6:36125455 | 30030 | 6:36143409 | 30031 | 6:36145256 | 30032 | 6:36147418 | 30033 | 6:36148375 |
| 30034 | 6:36149041 | 30035 | 6:36150876 | 30036 | 6:36151325 | 30037 | 6:36153013 | 30038 | 6:36156606 |
| 30039 | 6:36156894 | 30040 | 6:36157622 | 30041 | 6:36157988 | 30042 | 6:36158361 | 30043 | 6:36158500 |
| 30044 | 6:36159608 | 30045 | 6:36161275 | 30046 | 6:36161377 | 30047 | 6:36161627 | 30048 | 6:36161635 |
| 30049 | 6:36162952 | 30050 | 6:36163342 | 30051 | 6:36163430 | 30052 | 6:36164022 | 30053 | 6:36164566 |
| 30054 | 6:36165003 | 30055 | 6:36165139 | 30056 | 6:36165152 | 30057 | 6:36165220 | 30058 | 6:36165307 |
| 30059 | 6:36166110 | 30060 | 6:36166180 | 30061 | 6:36166207 | 30062 | 6:36166505 | 30063 | 6:36166793 |
| 30064 | 6:36166797 | 30065 | 6:36167327 | 30066 | 6:36167830 | 30067 | 6:36168126 | 30068 | 6:36168722 |
| 30069 | 6:36169714 | 30070 | 6:36169794 | 30071 | 6:36170135 | 30072 | 6:36170201 | 30073 | 6:36171686 |
| 30074 | 6:36172372 | 30075 | 6:36177201 | 30076 | 6:36180645 | 30077 | 6:36181021 | 30078 | 6:36182794 |
| 30079 | 6:36183913 | 30080 | 6:36186583 | 30081 | 6:36191256 | 30082 | 6:36198702 | 30083 | 6:36204036 |
| 30084 | 6:36211597 | 30085 | 6:36212186 | 30086 | 6:36219844 | 30087 | 6:36225618 | 30088 | 6:36236546 |
| 30089 | 6:36238601 | 30090 | 6:36238793 | 30091 | 6:36239080 | 30092 | 6:36239773 | 30093 | 6:36240264 |
| 30094 | 6:36241246 | 30095 | 6:36246325 | 30096 | 6:36248262 | 30097 | 6:36249143 | 30098 | 6:36250084 |
| 30099 | 6:36254264 | 30100 | 6:36255334 | 30101 | 6:36256227 | 30102 | 6:36256264 | 30103 | 6:36256316 |
| 30104 | 6:36261278 | 30105 | 6:36262429 | 30106 | 6:36263719 | 30107 | 6:36264671 | 30108 | 6:36264792 |
| 30109 | 6:36265379 | 30110 | 6:36272248 | 30111 | 6:36273670 | 30112 | 6:36277826 | 30113 | 6:36280253 |
| 30114 | 6:36283110 | 30115 | 6:36283198 | 30116 | 6:36286200 | 30117 | 6:36292811 | 30118 | 6:36298957 |
| 30119 | 6:36299463 | 30120 | 6:36299867 | 30121 | 6:36300007 | 30122 | 6:36324361 | 30123 | 6:36333247 |
| 30124 | 6:36336622 | 30125 | 6:36336765 | 30126 | 6:36337366 | 30127 | 6:36340265 | 30128 | 6:36363919 |
| 30129 | 6:36365275 | 30131 | 6:36412092 | 30141 | 6:36436269 | 30142 | 6:36440409 | 30143 | 6:36441375 |
| 30144 | 6:36443819 | 30146 | 6:36445226 | 30149 | 6:36454421 | 30151 | 6:36457471 | 30152 | 6:36457509 |
| 30153 | 6:36458519 | 30154 | 6:36460784 | 30155 | 6:36461234 | 30156 | 6:36461574 | 30157 | 6:36463931 |
| 30158 | 6:36466353 | 30159 | 6:36466871 | 30161 | 6:36469381 | 30162 | 6:36469975 | 30163 | 6:36474704 |
| 30166 | 6:36478628 | 30167 | 6:36480305 | 30170 | 6:36482113 | 30172 | 6:36487754 | 30173 | 6:36489160 |
| 30176 | 6:36491203 | 30177 | 6:36491990 | 30178 | 6:36499104 | 30179 | 6:36499659 | 30180 | 6:36500389 |
| 30181 | 6:36501474 | 30182 | 6:36503509 | 30183 | 6:36506439 | 30184 | 6:36506901 | 30185 | 6:36509073 |
| 30186 | 6:36514666 | 30187 | 6:36514687 | 30188 | 6:36516071 | 30189 | 6:36518117 | 30190 | 6:36524445 |
| 30191 | 6:36524489 | 30192 | 6:36529972 | 30193 | 6:36530565 | 30194 | 6:36531251 | 30195 | 6:36531630 |
| 30197 | 6:36532751 | 30198 | 6:36533529 | 30199 | 6:36533756 | 30200 | 6:36533947 | 30201 | 6:36534891 |
| 30202 | 6:36535012 | 30203 | 6:36535227 | 30204 | 6:36535356 | 30205 | 6:36535863 | 30206 | 6:36535904 |
| 30207 | 6:36536565 | 30208 | 6:36537055 | 30209 | 6:36537210 | 30210 | 6:36537223 | 30211 | 6:36537284 |
| 30212 | 6:36537515 | 30213 | 6:36537657 | 30214 | 6:36537708 | 30215 | 6:36538044 | 30216 | 6:36538107 |
| 30217 | 6:36538113 | 30218 | 6:36538805 | 30219 | 6:36538827 | 30220 | 6:36539051 | 30221 | 6:36539508 |
| 30222 | 6:36539630 | 30223 | 6:36540193 | 30224 | 6:36540734 | 30225 | 6:36540815 | 30226 | 6:36543846 |
| 30227 | 6:36544549 | 30228 | 6:36544681 | 30229 | 6:36544687 | 30230 | 6:36544786 | 30231 | 6:36544914 |
| 30232 | 6:36545202 | 30233 | 6:36545304 | 30235 | 6:36547031 | 30236 | 6:36547078 | 30237 | 6:36547888 |
| 30238 | 6:36547972 | 30239 | 6:36548012 | 30240 | 6:36548205 | 30241 | 6:36548953 | 30242 | 6:36549261 |
| 30243 | 6:36549683 | 30244 | 6:36550069 | 30245 | 6:36550199 | 30246 | 6:36551621 | 30247 | 6:36552037 |
| 30248 | 6:36552742 | 30249 | 6:36552915 | 30252 | 6:36556271 | 30253 | 6:36556442 | 30254 | 6:36557671 |
| 30256 | 6:36561952 | 30258 | 6:36567616 | 30262 | 6:36569777 | 30263 | 6:36571430 | 30265 | 6:36594692 |
| 30266 | 6:36598347 | 30267 | 6:36599192 | 30268 | 6:36602971 | 30269 | 6:36603958 | 30270 | 6:36608521 |
| 30271 | 6:36608642 | 30272 | 6:36611873 | 30273 | 6:36612067 | 30274 | 6:36614641 | 30275 | 6:36617150 |
| 30276 | 6:36618712 | 30277 | 6:36619708 | 30278 | 6:36620654 | 30279 | 6:36621895 | 30280 | 6:36622608 |
| 30281 | 6:36624334 | 30282 | 6:36625203 | 30283 | 6:36625485 | 30285 | 6:36626119 | 30286 | 6:36626509 |
| 30287 | 6:36629387 | 30288 | 6:36631261 | 30289 | 6:36632276 | 30290 | 6:36632779 | 30291 | 6:36633630 |
| 30292 | 6:36633929 | 30293 | 6:36634989 | 30294 | 6:36635404 | 30295 | 6:36635605 | 30296 | 6:36635939 |
| 30297 | 6:36638521 | 30298 | 6:36638963 | 30299 | 6:36639106 | 30300 | 6:36642083 | 30301 | 6:36642499 |
| 30302 | 6:36643100 | 30303 | 6:36643378 | 30304 | 6:36644151 | 30305 | 6:36645330 | 30306 | 6:36646194 |
| 30307 | 6:36646596 | 30308 | 6:36648068 | 30309 | 6:36648160 | 30310 | 6:36653563 | 30311 | 6:36657483 |
| 30312 | 6:36664663 | 30313 | 6:36673216 | 30314 | 6:36679692 | 30316 | 6:36681772 | 30318 | 6:36695293 |
| 30319 | 6:36700131 | 30320 | 6:36700135 | 30321 | 6:36706370 | 30322 | 6:36720331 | 30324 | 6:36743854 |
| 30325 | 6:36773960 | 30326 | 6:36787210 | 30331 | 6:36812951 | 30332 | 6:36813102 | 30333 | 6:36813124 |
| 30334 | 6:36813128 | 30335 | 6:36813593 | 30336 | 6:36813633 | 30337 | 6:36814581 | 30338 | 6:36814786 |
| 30339 | 6:36815144 | 30340 | 6:36815200 | 30341 | 6:36815490 | 30342 | 6:36815725 | 30343 | 6:36815817 |
| 30344 | 6:36816067 | 30345 | 6:36816314 | 30346 | 6:36816419 | 30347 | 6:36817159 | 30348 | 6:36817266 |
| 30349 | 6:36817581 | 30350 | 6:36817596 | 30351 | 6:36817683 | 30352 | 6:36817713 | 30353 | 6:36817969 |
| 30354 | 6:36818237 | 30355 | 6:36818704 | 30356 | 6:36818808 | 30357 | 6:36819594 | 30358 | 6:36819695 |
| 30359 | 6:36819751 | 30360 | 6:36820074 | 30361 | 6:36820138 | 30362 | 6:36820361 | 30363 | 6:36820770 |
| 30364 | 6:36821432 | 30365 | 6:36821474 | 30366 | 6:36822273 | 30367 | 6:36822526 | 30368 | 6:36822568 |
| 30369 | 6:36822744 | 30370 | 6:36823102 | 30371 | 6:36823606 | 30372 | 6:36825509 | 30373 | 6:36830103 |
| 30374 | 6:36830934 | 30375 | 6:36831012 | 30376 | 6:36831131 | 30377 | 6:36831274 | 30378 | 6:36831813 |
| 30379 | 6:36831871 | 30380 | 6:36834692 | 30381 | 6:36835108 | 30391 | 6:36949956 | 30392 | 6:36952579 |
| 30393 | 6:36954127 | 30395 | 6:36959305 | 30396 | 6:36960146 | 30397 | 6:36970451 | 30398 | 6:36978207 |
| 30399 | 6:36978216 | 30400 | 6:36978361 | 30401 | 6:36978371 | 30402 | 6:36978422 | 30403 | 6:36978431 |
| 30404 | 6:36978698 | 30405 | 6:36978703 | 30406 | 6:36978958 | 30407 | 6:36979025 | 30408 | 6:36979081 |
| 30409 | 6:36979093 | 30410 | 6:36979116 | 30411 | 6:36979134 | 30412 | 6:36979146 | 30413 | 6:36981664 |
| 30414 | 6:36981815 | 30415 | 6:36985783 | 30416 | 6:36985804 | 30417 | 6:36995904 | 30419 | 6:37010986 |
| 30420 | 6:37011036 | 30421 | 6:37016748 | 30422 | 6:37017628 | 30423 | 6:37017833 | 30424 | 6:37018373 |
| 30425 | 6:37018495 | 30426 | 6:37018672 | 30427 | 6:37018745 | 30428 | 6:37018878 | 30429 | 6:37019035 |
| 30430 | 6:37019263 | 30431 | 6:37019274 | 30432 | 6:37019373 | 30433 | 6:37019415 | 30434 | 6:37019464 |
| 30435 | 6:37019512 | 30436 | 6:37019519 | 30437 | 6:37019606 | 30438 | 6:37020100 | 30439 | 6:37020244 |
| 30440 | 6:37020255 | 30441 | 6:37020456 | 30442 | 6:37020574 | 30443 | 6:37020591 | 30444 | 6:37020608 |
| 30445 | 6:37021123 | 30446 | 6:37021154 | 30447 | 6:37021520 | 30448 | 6:37021604 | 30449 | 6:37021714 |
| 30450 | 6:37021968 | 30451 | 6:37022137 | 30452 | 6:37022181 | 30453 | 6:37022238 | 30454 | 6:37023645 |
| 30460 | 6:37030480 | 30461 | 6:37032906 | 30463 | 6:37034096 | 30464 | 6:37034286 | 30465 | 6:37034823 |
| 30466 | 6:37034912 | 30467 | 6:37035186 | 30469 | 6:37038017 | 30470 | 6:37044068 | 30473 | 6:37061527 |
| 30474 | 6:37074563 | 30475 | 6:37075910 | 30476 | 6:37078470 | 30477 | 6:37078665 | 30478 | 6:37078781 |
| 30479 | 6:37079090 | 30480 | 6:37079147 | 30481 | 6:37079324 | 30482 | 6:37079422 | 30483 | 6:37079559 |
| 30484 | 6:37079653 | 30485 | 6:37079666 | 30486 | 6:37080062 | 30487 | 6:37080097 | 30488 | 6:37080210 |
| 30489 | 6:37080486 | 30490 | 6:37080919 | 30491 | 6:37080936 | 30492 | 6:37081034 | 30493 | 6:37081117 |
| 30494 | 6:37081212 | 30495 | 6:37081319 | 30496 | 6:37081745 | 30497 | 6:37081947 | 30498 | 6:37082116 |
| 30499 | 6:37082537 | 30500 | 6:37082542 | 30501 | 6:37082592 | 30502 | 6:37082641 | 30503 | 6:37082764 |
| 30504 | 6:37082830 | 30505 | 6:37083023 | 30506 | 6:37083086 | 30507 | 6:37083196 | 30508 | 6:37083881 |
| 30509 | 6:37084518 | 30510 | 6:37084543 | 30511 | 6:37084979 | 30512 | 6:37084987 | 30513 | 6:37084991 |
| 30514 | 6:37085423 | 30515 | 6:37085516 | 30516 | 6:37087274 | 30517 | 6:37087642 | 30518 | 6:37087987 |
| 30519 | 6:37087999 | 30520 | 6:37088359 | 30521 | 6:37088506 | 30522 | 6:37088510 | 30523 | 6:37088831 |
| 30524 | 6:37088835 | 30525 | 6:37088955 | 30526 | 6:37089045 | 30527 | 6:37089108 | 30528 | 6:37089125 |
| 30529 | 6:37089202 | 30530 | 6:37089209 | 30531 | 6:37089234 | 30532 | 6:37089651 | 30533 | 6:37089673 |
| 30534 | 6:37089763 | 30535 | 6:37089819 | 30536 | 6:37089854 | 30537 | 6:37089926 | 30538 | 6:37090001 |
| 30539 | 6:37090127 | 30540 | 6:37090147 | 30541 | 6:37090190 | 30542 | 6:37090229 | 30543 | 6:37090257 |
| 30544 | 6:37090343 | 30545 | 6:37090838 | 30546 | 6:37090865 | 30547 | 6:37091359 | 30548 | 6:37091365 |
| 30549 | 6:37091616 | 30550 | 6:37091767 | 30551 | 6:37092094 | 30552 | 6:37092116 | 30553 | 6:37092227 |
| 30554 | 6:37092573 | 30555 | 6:37092869 | 30556 | 6:37092904 | 30557 | 6:37092936 | 30558 | 6:37093073 |
| 30559 | 6:37093934 | 30560 | 6:37093949 | 30561 | 6:37094212 | 30562 | 6:37094603 | 30563 | 6:37094693 |
| 30564 | 6:37094789 | 30565 | 6:37094827 | 30566 | 6:37095299 | 30567 | 6:37095517 | 30568 | 6:37096027 |
| 30569 | 6:37096074 | 30570 | 6:37096486 | 30571 | 6:37096495 | 30572 | 6:37096649 | 30573 | 6:37096879 |
| 30574 | 6:37096959 | 30575 | 6:37097509 | 30576 | 6:37097522 | 30577 | 6:37097635 | 30578 | 6:37097772 |
| 30579 | 6:37097995 | 30580 | 6:37098052 | 30581 | 6:37098220 | 30582 | 6:37098309 | 30583 | 6:37098640 |
| 30584 | 6:37098716 | 30585 | 6:37098782 | 30586 | 6:37098831 | 30587 | 6:37098872 | 30588 | 6:37099284 |
| 30589 | 6:37099535 | 30590 | 6:37099560 | 30591 | 6:37099942 | 30592 | 6:37099978 | 30593 | 6:37099983 |
| 30594 | 6:37100199 | 30595 | 6:37100203 | 30596 | 6:37100223 | 30597 | 6:37100242 | 30598 | 6:37100749 |
| 30599 | 6:37100852 | 30600 | 6:37100906 | 30601 | 6:37100924 | 30602 | 6:37101114 | 30603 | 6:37101213 |
| 30604 | 6:37101414 | 30605 | 6:37101664 | 30606 | 6:37101691 | 30607 | 6:37104751 | 30608 | 6:37104774 |
| 30609 | 6:37104851 | 30610 | 6:37104926 | 30611 | 6:37104993 | 30612 | 6:37105044 | 30613 | 6:37105252 |
| 30614 | 6:37105257 | 30615 | 6:37106212 | 30616 | 6:37107589 | 30617 | 6:37107774 | 30618 | 6:37108028 |
| 30619 | 6:37108237 | 30620 | 6:37112827 | 30621 | 6:37112864 | 30622 | 6:37116160 | 30623 | 6:37116221 |
| 30624 | 6:37116481 | 30625 | 6:37116730 | 30626 | 6:37117771 | 30627 | 6:37117988 | 30628 | 6:37120342 |
| 30630 | 6:37123023 | 30631 | 6:37128820 | 30634 | 6:37136040 | 30635 | 6:37136706 | 30636 | 6:37136794 |
| 30637 | 6:37138117 | 30638 | 6:37138139 | 30639 | 6:37138262 | 30640 | 6:37138535 | 30641 | 6:37139425 |
| 30642 | 6:37140287 | 30643 | 6:37142174 | 30644 | 6:37142953 | 30645 | 6:37144122 | 30646 | 6:37147544 |
| 30647 | 6:37147627 | 30648 | 6:37149236 | 30649 | 6:37149945 | 30650 | 6:37150579 | 30651 | 6:37151586 |
| 30652 | 6:37151618 | 30654 | 6:37152332 | 30656 | 6:37153197 | 30657 | 6:37153310 | 30658 | 6:37153398 |
| 30659 | 6:37153558 | 30660 | 6:37153831 | 30661 | 6:37155597 | 30662 | 6:37156064 | 30663 | 6:37156337 |
| 30664 | 6:37156677 | 30665 | 6:37156889 | 30666 | 6:37157333 | 30667 | 6:37157660 | 30668 | 6:37158136 |
| 30669 | 6:37158289 | 30670 | 6:37158384 | 30671 | 6:37159298 | 30672 | 6:37159341 | 30673 | 6:37159410 |
| 30674 | 6:37159427 | 30675 | 6:37159591 | 30676 | 6:37159718 | 30677 | 6:37159778 | 30678 | 6:37159892 |
| 30679 | 6:37160005 | 30680 | 6:37160134 | 30681 | 6:37160139 | 30682 | 6:37160559 | 30683 | 6:37161039 |
| 30684 | 6:37161146 | 30685 | 6:37161408 | 30686 | 6:37161619 | 30687 | 6:37161712 | 30688 | 6:37162703 |
| 30689 | 6:37162813 | 30690 | 6:37162822 | 30691 | 6:37163101 | 30692 | 6:37163223 | 30693 | 6:37163776 |
| 30694 | 6:37163934 | 30695 | 6:37164094 | 30696 | 6:37164218 | 30697 | 6:37164439 | 30698 | 6:37164529 |
| 30699 | 6:37164825 | 30700 | 6:37165013 | 30701 | 6:37165306 | 30702 | 6:37165536 | 30703 | 6:37165545 |
| 30704 | 6:37165588 | 30705 | 6:37165831 | 30706 | 6:37165836 | 30707 | 6:37166157 | 30708 | 6:37166449 |
| 30709 | 6:37166482 | 30710 | 6:37167166 | 30711 | 6:37167349 | 30712 | 6:37167472 | 30713 | 6:37167487 |
| 30714 | 6:37167778 | 30715 | 6:37167868 | 30716 | 6:37167910 | 30717 | 6:37167946 | 30718 | 6:37168208 |
| 30719 | 6:37168379 | 30720 | 6:37168414 | 30721 | 6:37168500 | 30722 | 6:37168914 | 30723 | 6:37168959 |
| 30724 | 6:37169024 | 30725 | 6:37169066 | 30726 | 6:37169247 | 30727 | 6:37169728 | 30728 | 6:37169746 |
| 30729 | 6:37169824 | 30730 | 6:37171398 | 30731 | 6:37171831 | 30732 | 6:37172063 | 30733 | 6:37172194 |
| 30734 | 6:37172300 | 30736 | 6:37173048 | 30737 | 6:37173169 | 30738 | 6:37173382 | 30739 | 6:37174433 |
| 30740 | 6:37174739 | 30741 | 6:37174812 | 30742 | 6:37174838 | 30743 | 6:37174847 | 30744 | 6:37174944 |
| 30745 | 6:37175216 | 30746 | 6:37175405 | 30747 | 6:37175422 | 30749 | 6:37175709 | 30751 | 6:37175915 |
| 30752 | 6:37175930 | 30753 | 6:37175957 | 30754 | 6:37176060 | 30755 | 6:37176229 | 30756 | 6:37176528 |
| 30758 | 6:37176686 | 30759 | 6:37177441 | 30760 | 6:37177466 | 30761 | 6:37177638 | 30762 | 6:37177788 |
| 30763 | 6:37177792 | 30764 | 6:37177809 | 30765 | 6:37178122 | 30766 | 6:37178336 | 30767 | 6:37178722 |
| 30768 | 6:37179535 | 30769 | 6:37180932 | 30770 | 6:37180966 | 30771 | 6:37181059 | 30772 | 6:37181087 |
| 30773 | 6:37181206 | 30774 | 6:37181374 | 30775 | 6:37181533 | 30776 | 6:37181894 | 30777 | 6:37182068 |
| 30778 | 6:37182373 | 30779 | 6:37182427 | 30780 | 6:37182621 | 30782 | 6:37182810 | 30783 | 6:37182983 |
| 30784 | 6:37183090 | 30785 | 6:37183609 | 30786 | 6:37183649 | 30789 | 6:37185316 | 30790 | 6:37188198 |
| 30793 | 6:37191616 | 30795 | 6:37193241 | 30796 | 6:37195555 | 30800 | 6:37211057 | 30801 | 6:37214068 |
| 30802 | 6:37214389 | 30803 | 6:37214736 | 30804 | 6:37216084 | 30805 | 6:37216293 | 30806 | 6:37217600 |
| 30807 | 6:37218354 | 30808 | 6:37218423 | 30809 | 6:37219372 | 30811 | 6:37221337 | 30812 | 6:37221507 |
| 30813 | 6:37221776 | 30814 | 6:37222424 | 30815 | 6:37225729 | 30816 | 6:37226016 | 30817 | 6:37226725 |
| 30819 | 6:37228661 | 30821 | 6:37231769 | 30822 | 6:37232090 | 30823 | 6:37232142 | 30824 | 6:37232577 |
| 30825 | 6:37233429 | 30826 | 6:37233659 | 30827 | 6:37234042 | 30828 | 6:37234954 | 30831 | 6:37236226 |
| 30832 | 6:37237969 | 30833 | 6:37238128 | 30834 | 6:37238402 | 30835 | 6:37238486 | 30836 | 6:37238502 |
| 30837 | 6:37238713 | 30838 | 6:37238946 | 30839 | 6:37241792 | 30840 | 6:37241812 | 30841 | 6:37243159 |
| 30842 | 6:37243373 | 30843 | 6:37243677 | 30845 | 6:37244804 | 30846 | 6:37245026 | 30847 | 6:37245138 |
| 30849 | 6:37247874 | 30850 | 6:37248754 | 30853 | 6:37251182 | 30854 | 6:37252978 | 30855 | 6:37255945 |
| 30860 | 6:37263319 | 30861 | 6:37264863 | 30862 | 6:37265148 | 30863 | 6:37266735 | 30866 | 6:37270930 |
| 30868 | 6:37273774 | 30869 | 6:37278799 | 30870 | 6:37281308 | 30871 | 6:37281335 | 30872 | 6:37282356 |
| 30873 | 6:37284874 | 30874 | 6:37285174 | 30875 | 6:37285822 | 30876 | 6:37285843 | 30877 | 6:37286325 |
| 30878 | 6:37286380 | 30880 | 6:37286722 | 30889 | 6:37289572 | 30904 | 6:37293699 | 30906 | 6:37294860 |
| 30907 | 6:37295049 | 30908 | 6:37295154 | 30909 | 6:37295245 | 30910 | 6:37295403 | 30911 | 6:37295803 |
| 30912 | 6:37296114 | 30913 | 6:37297426 | 30914 | 6:37297697 | 30915 | 6:37298251 | 30916 | 6:37298617 |
| 30917 | 6:37298725 | 30918 | 6:37298786 | 30919 | 6:37299349 | 30920 | 6:37299587 | 30921 | 6:37299855 |
| 30922 | 6:37299968 | 30923 | 6:37300447 | 30924 | 6:37300499 | 30925 | 6:37300590 | 30926 | 6:37300767 |
| 30927 | 6:37300810 | 30928 | 6:37300872 | 30929 | 6:37301720 | 30930 | 6:37301762 | 30931 | 6:37301893 |
| 30932 | 6:37301959 | 30933 | 6:37302011 | 30934 | 6:37302172 | 30935 | 6:37302478 | 30936 | 6:37302499 |
| 30937 | 6:37302730 | 30938 | 6:37303255 | 30939 | 6:37303264 | 30940 | 6:37303500 | 30941 | 6:37303542 |
| 30942 | 6:37303727 | 30943 | 6:37304109 | 30944 | 6:37304324 | 30945 | 6:37304493 | 30947 | 6:37304564 |
| 30948 | 6:37304601 | 30949 | 6:37304640 | 30950 | 6:37304825 | 30951 | 6:37305062 | 30952 | 6:37305466 |
| 30953 | 6:37305470 | 30954 | 6:37305490 | 30955 | 6:37305557 | 30956 | 6:37306944 | 30957 | 6:37306975 |
| 30958 | 6:37307238 | 30961 | 6:37310693 | 30962 | 6:37311375 | 30963 | 6:37311766 | 30964 | 6:37311816 |
| 30965 | 6:37311980 | 30966 | 6:37312071 | 30967 | 6:37312238 | 30968 | 6:37312270 | 30969 | 6:37312326 |
| 30970 | 6:37312692 | 30971 | 6:37313215 | 30972 | 6:37313503 | 30973 | 6:37313663 | 30974 | 6:37313742 |
| 30975 | 6:37313954 | 30976 | 6:37314022 | 30977 | 6:37314183 | 30978 | 6:37314291 | 30979 | 6:37315223 |
| 30980 | 6:37315480 | 30981 | 6:37315752 | 30982 | 6:37315774 | 30983 | 6:37316117 | 30984 | 6:37316265 |
| 30985 | 6:37316471 | 30986 | 6:37316594 | 30987 | 6:37316659 | 30988 | 6:37316928 | 30989 | 6:37317087 |
| 30990 | 6:37317153 | 30991 | 6:37317167 | 30992 | 6:37317341 | 30993 | 6:37317695 | 30994 | 6:37318285 |
| 30995 | 6:37318305 | 30996 | 6:37318658 | 30997 | 6:37318743 | 30998 | 6:37318847 | 30999 | 6:37319284 |
| 31000 | 6:37319902 | 31001 | 6:37320093 | 31002 | 6:37321012 | 31003 | 6:37321049 | 31004 | 6:37321441 |
| 31005 | 6:37321485 | 31006 | 6:37322361 | 31007 | 6:37322770 | 31008 | 6:37323838 | 31009 | 6:37323843 |
| 31010 | 6:37324718 | 31011 | 6:37325097 | 31012 | 6:37325189 | 31013 | 6:37326965 | 31014 | 6:37327393 |
| 31015 | 6:37327644 | 31016 | 6:37328014 | 31017 | 6:37328069 | 31018 | 6:37328528 | 31019 | 6:37328898 |
| 31020 | 6:37329042 | 31021 | 6:37329061 | 31022 | 6:37329319 | 31023 | 6:37329413 | 31024 | 6:37329526 |
| 31025 | 6:37329583 | 31027 | 6:37329948 | 31028 | 6:37330186 | 31029 | 6:37330198 | 31031 | 6:37330365 |
| 31034 | 6:37330605 | 31035 | 6:37330677 | 31036 | 6:37331712 | 31037 | 6:37331772 | 31038 | 6:37331921 |
| 31039 | 6:37332914 | 31040 | 6:37333089 | 31041 | 6:37333257 | 31042 | 6:37333646 | 31043 | 6:37335096 |
| 31044 | 6:37335574 | 31045 | 6:37335884 | 31047 | 6:37336176 | 31048 | 6:37336277 | 31049 | 6:37336431 |
| 31050 | 6:37337044 | 31051 | 6:37337343 | 31053 | 6:37337910 | 31054 | 6:37340194 | 31055 | 6:37340475 |
| 31056 | 6:37340937 | 31057 | 6:37341535 | 31058 | 6:37342568 | 31059 | 6:37342705 | 31060 | 6:37343364 |
| 31061 | 6:37343379 | 31062 | 6:37344246 | 31063 | 6:37344303 | 31064 | 6:37344897 | 31065 | 6:37345259 |
| 31066 | 6:37345508 | 31067 | 6:37345931 | 31068 | 6:37346124 | 31069 | 6:37346442 | 31070 | 6:37347130 |
| 31071 | 6:37349144 | 31072 | 6:37350316 | 31073 | 6:37350368 | 31074 | 6:37351499 | 31075 | 6:37351767 |
| 31076 | 6:37352145 | 31077 | 6:37352282 | 31078 | 6:37352820 | 31079 | 6:37353288 | 31080 | 6:37354945 |
| 31081 | 6:37355132 | 31082 | 6:37355369 | 31083 | 6:37356067 | 31084 | 6:37357055 | 31085 | 6:37358828 |
| 31086 | 6:37359765 | 31087 | 6:37360499 | 31088 | 6:37360685 | 31089 | 6:37361449 | 31090 | 6:37361578 |
| 31092 | 6:37363842 | 31093 | 6:37365265 | 31094 | 6:37365744 | 31095 | 6:37366868 | 31096 | 6:37368371 |
| 31097 | 6:37368652 | 31098 | 6:37368670 | 31100 | 6:37369380 | 31101 | 6:37370421 | 31103 | 6:37373261 |
| 31104 | 6:37373314 | 31105 | 6:37374314 | 31106 | 6:37374718 | 31107 | 6:37376009 | 31109 | 6:37377606 |
| 31110 | 6:37377857 | 31112 | 6:37378824 | 31114 | 6:37379507 | 31115 | 6:37379693 | 31116 | 6:37379729 |
| 31117 | 6:37379796 | 31118 | 6:37380161 | 31119 | 6:37380671 | 31120 | 6:37381106 | 31121 | 6:37381616 |
| 31125 | 6:37382177 | 31126 | 6:37383284 | 31128 | 6:37384356 | 31130 | 6:37390341 | 31131 | 6:37390788 |
| 31132 | 6:37391198 | 31133 | 6:37391915 | 31134 | 6:37392465 | 31135 | 6:37392540 | 31136 | 6:37392957 |
| 31137 | 6:37393564 | 31138 | 6:37393949 | 31140 | 6:37395313 | 31141 | 6:37395750 | 31142 | 6:37395838 |
| 31143 | 6:37396789 | 31144 | 6:37398199 | 31146 | 6:37399852 | 31147 | 6:37400741 | 31148 | 6:37400800 |
| 31149 | 6:37402511 | 31150 | 6:37402964 | 31152 | 6:37403224 | 31153 | 6:37406239 | 31154 | 6:37406485 |
| 31155 | 6:37406729 | 31156 | 6:37407653 | 31157 | 6:37412062 | 31158 | 6:37412935 | 31159 | 6:37414352 |
| 31160 | 6:37414365 | 31161 | 6:37414416 | 31162 | 6:37415367 | 31164 | 6:37416649 | 31165 | 6:37417046 |
| 31167 | 6:37418164 | 31168 | 6:37418421 | 31170 | 6:37418449 | 31171 | 6:37420252 | 31173 | 6:37423505 |
| 31174 | 6:37424248 | 31175 | 6:37425030 | 31176 | 6:37429071 | 31177 | 6:37429595 | 31180 | 6:37431101 |
| 31181 | 6:37432458 | 31182 | 6:37432854 | 31183 | 6:37433502 | 31184 | 6:37433926 | 31185 | 6:37434244 |
| 31186 | 6:37435861 | 31187 | 6:37436146 | 31188 | 6:37436347 | 31189 | 6:37436731 | 31190 | 6:37437130 |
| 31191 | 6:37437538 | 31193 | 6:37438248 | 31194 | 6:37438530 | 31195 | 6:37438575 | 31196 | 6:37438599 |
| 31197 | 6:37438993 | 31198 | 6:37439190 | 31200 | 6:37440955 | 31201 | 6:37441274 | 31202 | 6:37441899 |
| 31203 | 6:37442320 | 31204 | 6:37444900 | 31205 | 6:37446552 | 31206 | 6:37448497 | 31207 | 6:37449358 |
| 31208 | 6:37449890 | 31209 | 6:37450921 | 31210 | 6:37453215 | 31211 | 6:37456193 | 31213 | 6:37461999 |
| 31214 | 6:37463282 | 31215 | 6:37463709 | 31217 | 6:37466194 | 31218 | 6:37473453 | 31219 | 6:37477629 |
| 31220 | 6:37479269 | 31221 | 6:37480277 | 31223 | 6:37483537 | 31224 | 6:37489614 | 31226 | 6:37493760 |
| 31227 | 6:37495347 | 31228 | 6:37500943 | 31230 | 6:37505093 | 31231 | 6:37508184 | 31232 | 6:37511610 |
| 31233 | 6:37512672 | 31234 | 6:37512811 | 31235 | 6:37513645 | 31236 | 6:37520035 | 31237 | 6:37521235 |
| 31238 | 6:37522190 | 31239 | 6:37522580 | 31240 | 6:37526812 | 31241 | 6:37527163 | 31242 | 6:37529800 |
| 31244 | 6:37543134 | 31245 | 6:37547826 | 31247 | 6:37559117 | 31250 | 6:37564896 | 31251 | 6:37566268 |
| 31253 | 6:37566504 | 31255 | 6:37573615 | 31257 | 6:37578899 | 31258 | 6:37579956 | 31259 | 6:37580857 |
| 31260 | 6:37585527 | 31261 | 6:37587677 | 31264 | 6:37591326 | 31266 | 6:37592943 | 31267 | 6:37595517 |
| 31269 | 6:37599145 | 31270 | 6:37599699 | 31271 | 6:37602013 | 31272 | 6:37604468 | 31273 | 6:37606625 |
| 31274 | 6:37607139 | 31275 | 6:37608496 | 31276 | 6:37608504 | 31277 | 6:37614039 | 31278 | 6:37614179 |
| 31279 | 6:37617530 | 31280 | 6:37624003 | 31281 | 6:37625923 | 31282 | 6:37627776 | 31283 | 6:37633434 |
| 31284 | 6:37636883 | 31285 | 6:37636900 | 31286 | 6:37637484 | 31290 | 6:37641448 | 31293 | 6:37644691 |
| 31294 | 6:37644915 | 31295 | 6:37645671 | 31296 | 6:37646000 | 31297 | 6:37646499 | 31298 | 6:37646597 |
| 31299 | 6:37652729 | 31300 | 6:37658289 | 31302 | 6:37664673 | 31303 | 6:37666279 | 31304 | 6:37669453 |
| 31305 | 6:37674317 | 31306 | 6:37675400 | 31307 | 6:37679238 | 31308 | 6:37681968 | 31309 | 6:37683699 |
| 31310 | 6:37687844 | 31312 | 6:37690283 | 31313 | 6:37690852 | 31314 | 6:37696619 | 31315 | 6:37697757 |
| 31316 | 6:37698657 | 31317 | 6:37699059 | 31318 | 6:37703461 | 31319 | 6:37704637 | 31320 | 6:37706431 |
| 31321 | 6:37706528 | 31322 | 6:37708048 | 31323 | 6:37708742 | 31324 | 6:37708953 | 31325 | 6:37709524 |
| 31327 | 6:37712519 | 31328 | 6:37712749 | 31329 | 6:37713097 | 31330 | 6:37713686 | 31331 | 6:37713889 |
| 31332 | 6:37719635 | 31333 | 6:37719804 | 31334 | 6:37720524 | 31335 | 6:37727280 | 31336 | 6:37728142 |
| 31337 | 6:37731195 | 31338 | 6:37736960 | 31339 | 6:37738532 | 31340 | 6:37740667 | 31341 | 6:37741893 |
| 31342 | 6:37742839 | 31343 | 6:37743712 | 31344 | 6:37749840 | 31345 | 6:37749955 | 31346 | 6:37751452 |
| 31347 | 6:37752740 | 31348 | 6:37760883 | 31349 | 6:37772675 | 31350 | 6:37773321 | 31351 | 6:37773355 |
| 31352 | 6:37774136 | 31354 | 6:37775491 | 31355 | 6:37775624 | 31356 | 6:37775822 | 31357 | 6:37776401 |
| 31358 | 6:37776428 | 31359 | 6:37776454 | 31360 | 6:37776464 | 31362 | 6:37777056 | 31363 | 6:37777185 |
| 31364 | 6:37778681 | 31365 | 6:37778924 | 31367 | 6:37797233 | 31368 | 6:37802327 | 31369 | 6:37803937 |
| 31370 | 6:37803941 | 31372 | 6:37804729 | 31373 | 6:37805097 | 31374 | 6:37806070 | 31376 | 6:37809966 |
| 31377 | 6:37810505 | 31378 | 6:37810779 | 31379 | 6:37811541 | 31380 | 6:37811600 | 31381 | 6:37811808 |
| 31382 | 6:37811947 | 31383 | 6:37812109 | 31384 | 6:37812521 | 31385 | 6:37813026 | 31386 | 6:37813294 |
| 31387 | 6:37813424 | 31388 | 6:37813572 | 31389 | 6:37813638 | 31390 | 6:37813728 | 31391 | 6:37813964 |
| 31392 | 6:37814265 | 31393 | 6:37814928 | 31394 | 6:37815457 | 31395 | 6:37815489 | 31396 | 6:37815615 |
| 31397 | 6:37815806 | 31398 | 6:37815918 | 31399 | 6:37816011 | 31400 | 6:37816192 | 31401 | 6:37816231 |
| 31402 | 6:37816300 | 31403 | 6:37817310 | 31405 | 6:37817589 | 31406 | 6:37817855 | 31407 | 6:37817875 |
| 31408 | 6:37817910 | 31409 | 6:37818107 | 31410 | 6:37818111 | 31411 | 6:37818195 | 31412 | 6:37818372 |
| 31413 | 6:37819224 | 31414 | 6:37821523 | 31415 | 6:37821595 | 31416 | 6:37821964 | 31417 | 6:37830099 |
| 31418 | 6:37831626 | 31419 | 6:37833439 | 31420 | 6:37838622 | 31421 | 6:37842392 | 31422 | 6:37895331 |
| 31423 | 6:37902755 | 31424 | 6:37905296 | 31425 | 6:37905467 | 31426 | 6:37907357 | 31427 | 6:37908478 |
| 31428 | 6:37908507 | 31429 | 6:37908679 | 31430 | 6:37908861 | 31431 | 6:37908962 | 31432 | 6:37909143 |
| 31433 | 6:37910276 | 31434 | 6:37910464 | 31435 | 6:37910478 | 31436 | 6:37910866 | 31437 | 6:37911814 |
| 31438 | 6:37912116 | 31439 | 6:37913125 | 31440 | 6:37913652 | 31441 | 6:37913727 | 31442 | 6:37913922 |
| 31443 | 6:37913940 | 31444 | 6:37914439 | 31446 | 6:37914513 | 31447 | 6:37914730 | 31448 | 6:37914816 |
| 31449 | 6:37914822 | 31450 | 6:37914832 | 31451 | 6:37915050 | 31452 | 6:37915308 | 31453 | 6:37916419 |
| 31454 | 6:37917366 | 31455 | 6:37917421 | 31456 | 6:37917796 | 31457 | 6:37918546 | 31458 | 6:37918824 |
| 31459 | 6:37919045 | 31460 | 6:37919095 | 31461 | 6:37919120 | 31462 | 6:37919520 | 31463 | 6:37919631 |
| 31464 | 6:37919661 | 31465 | 6:37919853 | 31466 | 6:37919862 | 31467 | 6:37920305 | 31468 | 6:37920538 |
| 31469 | 6:37920551 | 31470 | 6:37920666 | 31471 | 6:37921510 | 31472 | 6:37921603 | 31474 | 6:37921724 |
| 31476 | 6:37926600 | 31477 | 6:37926960 | 31478 | 6:37928156 | 31479 | 6:37928479 | 31480 | 6:37929065 |
| 31481 | 6:37929104 | 31482 | 6:37929184 | 31483 | 6:37929689 | 31484 | 6:37929786 | 31485 | 6:37930046 |
| 31487 | 6:37930293 | 31488 | 6:37930672 | 31489 | 6:37930781 | 31490 | 6:37930868 | 31491 | 6:37930889 |
| 31492 | 6:37931089 | 31493 | 6:37931216 | 31494 | 6:37931298 | 31495 | 6:37931621 | 31496 | 6:37931688 |
| 31497 | 6:37931726 | 31498 | 6:37931903 | 31499 | 6:37932170 | 31500 | 6:37932197 | 31501 | 6:37932241 |
| 31502 | 6:37932371 | 31503 | 6:37932377 | 31504 | 6:37932412 | 31505 | 6:37932434 | 31506 | 6:37932563 |
| 31508 | 6:37932772 | 31509 | 6:37932778 | 31510 | 6:37932960 | 31511 | 6:37933250 | 31512 | 6:37933352 |
| 31513 | 6:37933965 | 31514 | 6:37934068 | 31515 | 6:37934272 | 31516 | 6:37934993 | 31517 | 6:37935069 |
| 31518 | 6:37935108 | 31519 | 6:37935168 | 31520 | 6:37935527 | 31521 | 6:37935577 | 31522 | 6:37935748 |
| 31523 | 6:37936044 | 31524 | 6:37936087 | 31525 | 6:37936692 | 31526 | 6:37938294 | 31529 | 6:37942096 |
| 31530 | 6:37942170 | 31531 | 6:37942290 | 31532 | 6:37942944 | 31533 | 6:37943635 | 31534 | 6:37943860 |
| 31536 | 6:37944478 | 31537 | 6:37944482 | 31538 | 6:37944496 | 31539 | 6:37944573 | 31540 | 6:37944631 |
| 31541 | 6:37944681 | 31542 | 6:37944704 | 31543 | 6:37945333 | 31544 | 6:37945482 | 31545 | 6:37945820 |
| 31546 | 6:37945849 | 31547 | 6:37946012 | 31548 | 6:37946384 | 31549 | 6:37953254 | 31550 | 6:37953301 |
| 31551 | 6:37953591 | 31552 | 6:37954699 | 31553 | 6:37954789 | 31554 | 6:37954873 | 31555 | 6:37955372 |
| 31556 | 6:37956368 | 31557 | 6:37956390 | 31558 | 6:37959360 | 31559 | 6:37960313 | 31560 | 6:37961491 |
| 31561 | 6:37961553 | 31562 | 6:37962093 | 31563 | 6:37962887 | 31564 | 6:37963346 | 31565 | 6:37963525 |
| 31566 | 6:37965259 | 31567 | 6:37965489 | 31568 | 6:37966382 | 31569 | 6:37968595 | 31570 | 6:37970172 |
| 31571 | 6:37970860 | 31572 | 6:37971218 | 31573 | 6:37971544 | 31574 | 6:37971810 | 31575 | 6:37971997 |
| 31576 | 6:37972173 | 31577 | 6:37972335 | 31578 | 6:37972414 | 31579 | 6:37972642 | 31580 | 6:37972906 |
| 31581 | 6:37973011 | 31582 | 6:37973508 | 31583 | 6:37973804 | 31584 | 6:37974302 | 31586 | 6:37974407 |
| 31587 | 6:37974974 | 31588 | 6:37976818 | 31589 | 6:37978037 | 31590 | 6:37978995 | 31591 | 6:37979083 |
| 31592 | 6:37981093 | 31593 | 6:37981125 | 31594 | 6:37983153 | 31595 | 6:37984808 | 31596 | 6:37984948 |
| 31597 | 6:37985243 | 31598 | 6:37987337 | 31599 | 6:37987503 | 31600 | 6:37989831 | 31601 | 6:37990674 |
| 31602 | 6:37990808 | 31603 | 6:37991751 | 31604 | 6:37992856 | 31605 | 6:37993125 | 31606 | 6:37993517 |
| 31607 | 6:37993754 | 31608 | 6:37994046 | 31609 | 6:37996786 | 31610 | 6:37996859 | 31611 | 6:37997160 |
| 31612 | 6:37997516 | 31613 | 6:37998187 | 31614 | 6:37998452 | 31615 | 6:37998592 | 31616 | 6:37998656 |
| 31617 | 6:37998696 | 31618 | 6:37999740 | 31619 | 6:37999825 | 31620 | 6:37999842 | 31621 | 6:38000343 |
| 31622 | 6:38000353 | 31623 | 6:38001392 | 31624 | 6:38001622 | 31625 | 6:38002117 | 31626 | 6:38002596 |
| 31627 | 6:38004124 | 31628 | 6:38004325 | 31629 | 6:38005805 | 31630 | 6:38007975 | 31631 | 6:38014109 |
| 31632 | 6:38016521 | 31633 | 6:38017827 | 31634 | 6:38017866 | 31635 | 6:38021694 | 31636 | 6:38022226 |
| 31637 | 6:38022305 | 31638 | 6:38023218 | 31639 | 6:38023327 | 31640 | 6:38023545 | 31641 | 6:38023613 |
| 31642 | 6:38023673 | 31643 | 6:38023878 | 31644 | 6:38024011 | 31645 | 6:38024702 | 31646 | 6:38024768 |
| 31647 | 6:38025411 | 31648 | 6:38025684 | 31649 | 6:38025734 | 31650 | 6:38026545 | 31651 | 6:38027078 |
| 31652 | 6:38027106 | 31653 | 6:38027129 | 31654 | 6:38031018 | 31655 | 6:38032275 | 31656 | 6:38033896 |
| 31658 | 6:38035001 | 31659 | 6:38035414 | 31660 | 6:38035460 | 31661 | 6:38035561 | 31662 | 6:38035568 |
| 31663 | 6:38035625 | 31664 | 6:38035705 | 31665 | 6:38036061 | 31666 | 6:38036351 | 31667 | 6:38036588 |
| 31668 | 6:38036594 | 31669 | 6:38036635 | 31670 | 6:38036660 | 31671 | 6:38036882 | 31672 | 6:38037099 |
| 31673 | 6:38037535 | 31674 | 6:38037724 | 31675 | 6:38037768 | 31676 | 6:38038091 | 31677 | 6:38038189 |
| 31678 | 6:38038404 | 31679 | 6:38038608 | 31680 | 6:38039088 | 31681 | 6:38039492 | 31682 | 6:38040456 |
| 31683 | 6:38040488 | 31684 | 6:38040529 | 31685 | 6:38040769 | 31686 | 6:38040934 | 31687 | 6:38041234 |
| 31688 | 6:38041593 | 31689 | 6:38041633 | 31690 | 6:38041715 | 31691 | 6:38042205 | 31692 | 6:38042337 |
| 31693 | 6:38043977 | 31694 | 6:38044618 | 31695 | 6:38044876 | 31696 | 6:38044898 | 31697 | 6:38045727 |
| 31698 | 6:38046428 | 31699 | 6:38048735 | 31700 | 6:38051406 | 31701 | 6:38051509 | 31702 | 6:38052049 |
| 31703 | 6:38052245 | 31704 | 6:38054733 | 31706 | 6:38063490 | 31707 | 6:38064317 | 31708 | 6:38065703 |
| 31709 | 6:38066309 | 31710 | 6:38067289 | 31711 | 6:38067376 | 31712 | 6:38067855 | 31713 | 6:38068311 |
| 31714 | 6:38068956 | 31715 | 6:38069662 | 31716 | 6:38070361 | 31717 | 6:38070496 | 31718 | 6:38070882 |
| 31719 | 6:38071318 | 31720 | 6:38071414 | 31721 | 6:38071547 | 31722 | 6:38073914 | 31723 | 6:38076161 |
| 31724 | 6:38076905 | 31725 | 6:38077237 | 31726 | 6:38077323 | 31728 | 6:38078774 | 31729 | 6:38079456 |
| 31730 | 6:38079499 | 31731 | 6:38082355 | 31732 | 6:38085803 | 31733 | 6:38087237 | 31734 | 6:38087636 |
| 31735 | 6:38089862 | 31736 | 6:38094533 | 31737 | 6:38097987 | 31739 | 6:38107020 | 31740 | 6:38108493 |
| 31742 | 6:38114043 | 31743 | 6:38114047 | 31744 | 6:38114717 | 31745 | 6:38114803 | 31746 | 6:38114808 |
| 31747 | 6:38115023 | 31748 | 6:38118015 | 31749 | 6:38122903 | 31750 | 6:38123039 | 31751 | 6:38127700 |
| 31752 | 6:38131158 | 31753 | 6:38132133 | 31754 | 6:38132688 | 31755 | 6:38135706 | 31756 | 6:38137640 |
| 31757 | 6:38137799 | 31758 | 6:38138017 | 31759 | 6:38138089 | 31760 | 6:38139495 | 31761 | 6:38139563 |
| 31762 | 6:38139762 | 31763 | 6:38140108 | 31764 | 6:38140123 | 31765 | 6:38140536 | 31766 | 6:38140681 |
| 31767 | 6:38141284 | 31768 | 6:38141304 | 31769 | 6:38141629 | 31770 | 6:38141696 | 31771 | 6:38141833 |
| 31772 | 6:38142148 | 31773 | 6:38142807 | 31774 | 6:38142835 | 31775 | 6:38142840 | 31776 | 6:38144120 |
| 31777 | 6:38144195 | 31778 | 6:38144291 | 31779 | 6:38144317 | 31780 | 6:38144587 | 31781 | 6:38156378 |
| 31782 | 6:38156469 | 31783 | 6:38156591 | 31784 | 6:38157637 | 31785 | 6:38157684 | 31786 | 6:38158107 |
| 31787 | 6:38158113 | 31791 | 6:38199691 | 31792 | 6:38202499 | 31793 | 6:38211198 | 31795 | 6:38227779 |
| 31797 | 6:38237566 | 31798 | 6:38237695 | 31799 | 6:38247802 | 31800 | 6:38252776 | 31805 | 6:38278657 |
| 31806 | 6:38278666 | 31807 | 6:38278782 | 31808 | 6:38279030 | 31809 | 6:38281274 | 31814 | 6:38318628 |
| 31817 | 6:38375856 | 31820 | 6:38396789 | 31823 | 6:38409609 | 31825 | 6:38433243 | 31827 | 6:38499580 |
| 31828 | 6:38500871 | 31829 | 6:38506361 | 31830 | 6:38508464 | 31832 | 6:38519250 | 31833 | 6:38525790 |
| 31834 | 6:38535911 | 31835 | 6:38540318 | 31838 | 6:38541639 | 31840 | 6:38542150 | 31844 | 6:38543990 |
| 31848 | 6:38546646 | 31849 | 6:38548060 | 31850 | 6:38548198 | 31854 | 6:38549044 | 31856 | 6:38549206 |
| 31858 | 6:38549703 | 31859 | 6:38550162 | 31869 | 6:38553181 | 31881 | 6:38556220 | 31895 | 6:38562229 |
| 31896 | 6:38563063 | 31898 | 6:38565084 | 31899 | 6:38566242 | 31900 | 6:38570744 | 31901 | 6:38575762 |
| 31903 | 6:38584420 | 31904 | 6:38584686 | 31905 | 6:38588303 | 31906 | 6:38599441 | 31907 | 6:38600028 |
| 31908 | 6:38600235 | 31909 | 6:38602696 | 31910 | 6:38617268 | 31911 | 6:38617890 | 31912 | 6:38618593 |
| 31913 | 6:38619398 | 31914 | 6:38619754 | 31915 | 6:38619795 | 31916 | 6:38620014 | 31917 | 6:38620218 |
| 31918 | 6:38621930 | 31919 | 6:38624000 | 31920 | 6:38624126 | 31921 | 6:38644939 | 31923 | 6:38695951 |
| 31924 | 6:38697400 | 31928 | 6:38789984 | 31929 | 6:38798194 | 31933 | 6:38854750 | 31942 | 6:38907049 |
| 31943 | 6:38918524 | 31945 | 6:38919029 | 31946 | 6:38929469 | 31947 | 6:38933534 | 31948 | 6:38934821 |
| 31950 | 6:38963229 | 31951 | 6:38973635 | 31952 | 6:38973995 | 31953 | 6:38980873 | 31954 | 6:39030259 |
| 31956 | 6:39082331 | 31957 | 6:39109137 | 31962 | 6:39181262 | 31966 | 6:39254458 | 31968 | 6:39280700 |
| 31974 | 6:39437165 | 31977 | 6:39518939 | 31978 | 6:39532147 | 31979 | 6:39559751 | 31982 | 6:39574982 |
| 31984 | 6:39595691 | 31985 | 6:39595731 | 31988 | 6:39652421 | 31992 | 6:39721697 | 31994 | 6:39730068 |
| 31995 | 6:39733610 | 31996 | 6:39737879 | 31998 | 6:39755798 | 31999 | 6:39756006 | 32002 | 6:39760650 |
| 32003 | 6:39772834 | 32004 | 6:39772963 | 32005 | 6:39773133 | 32007 | 6:39797151 | 32009 | 6:39825526 |
| 32011 | 6:39858450 | 32012 | 6:39874821 | 32013 | 6:39885647 | 32026 | 6:40072649 | 32030 | 6:40130949 |
| 32032 | 6:40203593 | 32035 | 6:40239109 | 32036 | 6:40248052 | 32042 | 6:40338033 | 32047 | 6:40400197 |
| 32048 | 6:40432849 | 32049 | 6:40456680 | 32052 | 6:40565574 | 32055 | 6:40599155 | 32058 | 6:40636686 |
| 32059 | 6:40662051 | 32060 | 6:40672232 | 32061 | 6:40689118 | 32062 | 6:40696320 | 32085 | 6:40731922 |
| 32089 | 6:40758035 | 32093 | 6:40780850 | 32094 | 6:40808612 | 32098 | 6:40836283 | 32102 | 6:40911873 |
| 32103 | 6:40920367 | 32104 | 6:40921708 | 32105 | 6:40933517 | 32106 | 6:40935693 | 32107 | 6:40935713 |
| 32108 | 6:40935857 | 32109 | 6:40935890 | 32110 | 6:40936031 | 32111 | 6:40936207 | 32112 | 6:40936326 |
| 32113 | 6:40936831 | 32114 | 6:40936837 | 32115 | 6:40937031 | 32116 | 6:40937441 | 32117 | 6:40942588 |
| 32120 | 6:40984868 | 32121 | 6:40988645 | 32122 | 6:40988913 | 32123 | 6:40999090 | 32124 | 6:41003445 |
| 32125 | 6:41004894 | 32126 | 6:41029896 | 32127 | 6:41033792 | 32128 | 6:41049370 | 32129 | 6:41049496 |
| 32130 | 6:41049859 | 32131 | 6:41049914 | 32132 | 6:41050127 | 32133 | 6:41050140 | 32135 | 6:41050862 |
| 32136 | 6:41051120 | 32137 | 6:41053625 | 32138 | 6:41061635 | 32139 | 6:41073550 | 32140 | 6:41079462 |
| 32142 | 6:41088062 | 32143 | 6:41090326 | 32144 | 6:41096500 | 32145 | 6:41107449 | 32146 | 6:41107836 |
| 32147 | 6:41108192 | 32148 | 6:41108818 | 32149 | 6:41108912 | 32150 | 6:41108926 | 32151 | 6:41109137 |
| 32152 | 6:41109217 | 32153 | 6:41109350 | 32155 | 6:41150218 | 32156 | 6:41169318 | 32157 | 6:41170432 |
| 32158 | 6:41173967 | 32161 | 6:41185741 | 32162 | 6:41204988 | 32169 | 6:41306166 | 32170 | 6:41306273 |
| 32171 | 6:41306284 | 32172 | 6:41306424 | 32173 | 6:41308067 | 32174 | 6:41331258 | 32179 | 6:41577847 |
| 32180 | 6:41592300 | 32181 | 6:41677493 | 32188 | 6:41868518 | 32195 | 6:42004652 | 32203 | 6:42037316 |
| 32209 | 6:42163671 | 32216 | 6:42441818 | 32220 | 6:42507082 | 32221 | 6:42510330 | 32227 | 6:42559603 |
| 32229 | 6:42665842 | 32230 | 6:42672683 | 32231 | 6:42722868 | 32232 | 6:42724776 | 32233 | 6:42728138 |
| 32234 | 6:42741244 | 32235 | 6:42741447 | 32236 | 6:42758331 | 32241 | 6:42837669 | 32263 | 6:42974326 |
| 32284 | 6:43317249 | 32296 | 6:43494887 | 32298 | 6:43544441 | 32303 | 6:43601154 | 32309 | 6:43679917 |
| 32318 | 6:43822090 | 32319 | 6:43822403 | 32320 | 6:43829217 | 32323 | 6:43886543 | 32326 | 6:43944737 |
| 32330 | 6:43983354 | 32331 | 6:44003666 | 32332 | 6:44026498 | 32336 | 6:44072335 | 32339 | 6:44149286 |
| 32343 | 6:44276184 | 32360 | 6:44628911 | 32362 | 6:44678042 | 32363 | 6:44685469 | 32364 | 6:44686809 |
| 32365 | 6:44689379 | 32366 | 6:44690759 | 32368 | 6:44694506 | 32369 | 6:44694605 | 32370 | 6:44695255 |
| 32371 | 6:44695433 | 32372 | 6:44695563 | 32373 | 6:44695784 | 32374 | 6:44695872 | 32375 | 6:44695978 |
| 32376 | 6:44695987 | 32377 | 6:44696022 | 32378 | 6:44696578 | 32379 | 6:44696796 | 32380 | 6:44696931 |
| 32381 | 6:44696955 | 32382 | 6:44698160 | 32383 | 6:44704061 | 32384 | 6:44704410 | 32385 | 6:44705240 |
| 32386 | 6:44706199 | 32387 | 6:44706279 | 32388 | 6:44709016 | 32389 | 6:44709754 | 32390 | 6:44710003 |
| 32391 | 6:44710237 | 32392 | 6:44710415 | 32393 | 6:44710515 | 32394 | 6:44711099 | 32395 | 6:44712295 |
| 32396 | 6:44735374 | 32397 | 6:44736099 | 32398 | 6:44739055 | 32399 | 6:44742360 | 32400 | 6:44747933 |
| 32401 | 6:44748956 | 32403 | 6:44775326 | 32404 | 6:44777357 | 32405 | 6:44784742 | 32406 | 6:44787261 |
| 32407 | 6:44791513 | 32408 | 6:44791744 | 32409 | 6:44797875 | 32410 | 6:44809081 | 32411 | 6:44810108 |
| 32412 | 6:44818322 | 32413 | 6:44819710 | 32414 | 6:44820115 | 32415 | 6:44823097 | 32416 | 6:44825630 |
| 32418 | 6:44828213 | 32419 | 6:44831889 | 32420 | 6:44857705 | 32421 | 6:44864378 | 32422 | 6:44873514 |
| 32423 | 6:44873843 | 32424 | 6:44874186 | 32425 | 6:44877670 | 32426 | 6:44883344 | 32427 | 6:44893464 |
| 32428 | 6:44904257 | 32429 | 6:44905170 | 32430 | 6:44909660 | 32431 | 6:44914656 | 32432 | 6:44920744 |
| 32433 | 6:44931581 | 32434 | 6:44934298 | 32435 | 6:44934689 | 32437 | 6:44983137 | 32439 | 6:45018531 |
| 32440 | 6:45018882 | 32441 | 6:45029390 | 32442 | 6:45029425 | 32443 | 6:45029626 | 32444 | 6:45030486 |
| 32445 | 6:45030599 | 32446 | 6:45030709 | 32447 | 6:45030774 | 32448 | 6:45030789 | 32449 | 6:45031327 |
| 32450 | 6:45031721 | 32451 | 6:45031956 | 32452 | 6:45032017 | 32453 | 6:45032153 | 32454 | 6:45033264 |
| 32455 | 6:45037315 | 32456 | 6:45037561 | 32457 | 6:45037753 | 32458 | 6:45037798 | 32459 | 6:45038091 |
| 32460 | 6:45038647 | 32461 | 6:45038676 | 32462 | 6:45038869 | 32463 | 6:45040756 | 32464 | 6:45041754 |
| 32465 | 6:45043489 | 32466 | 6:45043654 | 32467 | 6:45043859 | 32468 | 6:45043984 | 32469 | 6:45044149 |
| 32470 | 6:45045596 | 32471 | 6:45048213 | 32472 | 6:45048323 | 32473 | 6:45049586 | 32474 | 6:45053716 |
| 32475 | 6:45054332 | 32476 | 6:45055326 | 32477 | 6:45057776 | 32478 | 6:45057805 | 32479 | 6:45058034 |
| 32480 | 6:45058273 | 32481 | 6:45077729 | 32485 | 6:45135036 | 32487 | 6:45183436 | 32489 | 6:45185710 |
| 32490 | 6:45190122 | 32491 | 6:45205382 | 32492 | 6:45213648 | 32497 | 6:45274484 | 32500 | 6:45322366 |
| 32502 | 6:45325845 | 32505 | 6:45364629 | 32518 | 6:45583636 | 32519 | 6:45595528 | 32526 | 6:45885499 |
| 32569 | 6:46705772 | 32576 | 6:46946921 | 32580 | 6:47016991 | 32586 | 6:47147481 | 32587 | 6:47164702 |
| 32591 | 6:47219584 | 32592 | 6:47256173 | 32594 | 6:47317499 | 32596 | 6:47365064 | 32599 | 6:47424087 |
| 32602 | 6:47570425 | 32614 | 6:47825353 | 32615 | 6:47843766 | 32616 | 6:47866852 | 32621 | 6:47989356 |
| 32623 | 6:48030364 | 32626 | 6:48149995 | 32648 | 6:48893818 | 32652 | 6:49019297 | 32655 | 6:49094600 |
| 32662 | 6:49295440 | 32679 | 6:49701147 | 32694 | 6:49848545 | 32696 | 6:49878456 | 32701 | 6:50009636 |
| 32703 | 6:50090470 | 32704 | 6:50126302 | 32709 | 6:50252554 | 32716 | 6:50385741 | 32717 | 6:50412585 |
| 32719 | 6:50455132 | 32724 | 6:50672369 | 32729 | 6:50779278 | 32731 | 6:50856440 | 32748 | 6:51325072 |
| 32750 | 6:51362285 | 32759 | 6:51740645 | 32778 | 6:52250315 | 32780 | 6:52275948 | 32781 | 6:52320441 |
| 32782 | 6:52339267 | 32783 | 6:52339325 | 32784 | 6:52347811 | 32785 | 6:52347899 | 32786 | 6:52347995 |
| 32787 | 6:52348073 | 32788 | 6:52348287 | 32789 | 6:52348292 | 32790 | 6:52348643 | 32791 | 6:52348674 |
| 32792 | 6:52349023 | 32793 | 6:52349157 | 32794 | 6:52349544 | 32795 | 6:52349562 | 32796 | 6:52349794 |
| 32797 | 6:52350003 | 32798 | 6:52350227 | 32799 | 6:52350240 | 32800 | 6:52351181 | 32801 | 6:52351388 |
| 32802 | 6:52352446 | 32817 | 6:52645662 | 32818 | 6:52671724 | 32825 | 6:52864424 | 32829 | 6:52954024 |
| 32831 | 6:53024244 | 32842 | 6:53220820 | 32846 | 6:53285747 | 32847 | 6:53314886 | 32850 | 6:53397301 |
| 32853 | 6:53471919 | 32862 | 6:53752557 | 32873 | 6:54004873 | 32874 | 6:54031927 | 32877 | 6:54086328 |
| 32879 | 6:54185052 | 32897 | 6:54783996 | 32913 | 6:55303674 | 32920 | 6:55450495 | 32941 | 6:55514144 |
| 32976 | 6:56317455 | 32994 | 6:56900473 | 33006 | 6:57179884 | 33012 | 6:57260380 | 33031 | 6:57842883 |
| 33035 | 6:57984344 | 33039 | 6:58077967 | 33066 | 6:58746181 | 33077 | 6:59130010 | 33095 | 6:59579872 |
| 33097 | 6:59716766 | 33111 | 6:59875075 | 33114 | 6:59907442 | 33122 | 6:60085027 | 33129 | 6:60257426 |
| 33137 | 6:60400323 | 33155 | 6:60802613 | 33168 | 6:61185257 | 33176 | 6:61390096 | 33183 | 6:61487026 |
| 33204 | 6:62234468 | 33223 | 6:62814916 | 33224 | 6:62862206 | 33225 | 6:62889960 | 33227 | 6:62925583 |
| 33238 | 6:63122811 | 33240 | 6:63170295 | 33242 | 6:63209301 | 33244 | 6:63244734 | 33260 | 6:63836353 |
| 33307 | 6:65456807 | 33308 | 6:65520848 | 33338 | 6:66507621 | 33350 | 6:66896326 | 33361 | 6:67256076 |
| 33376 | 6:67767947 | 33381 | 6:67991798 | 33394 | 6:68331252 | 33397 | 6:68416278 | 33418 | 6:68896760 |
| 33422 | 6:69030825 | 33433 | 6:69252356 | 33435 | 6:69346909 | 33444 | 6:69724839 | 33474 | 6:70673035 |
| 33478 | 6:70789469 | 33484 | 6:70978405 | 33516 | 6:72022899 | 33530 | 6:72349944 | 33534 | 6:72385883 |
| 33535 | 6:72419275 | 33544 | 6:72713559 | 33582 | 6:74091185 | 33584 | 6:74174913 | 33607 | 6:75066888 |
| 33608 | 6:75102649 | 33667 | 6:76880221 | 33698 | 6:77967685 | 33734 | 6:79446058 | 33751 | 6:80043649 |
| 33764 | 6:80444445 | 33798 | 6:81507132 | 33851 | 6:83098669 | 33875 | 6:83547735 | 33897 | 6:83979787 |
| 33921 | 6:84628765 | 33933 | 6:85067620 | 33936 | 6:85092523 | 33946 | 6:85177598 | 33953 | 6:85422786 |
| 33964 | 6:85548797 | 34000 | 6:86388176 | 34002 | 6:86478657 | 34055 | 6:87399244 | 34089 | 6:88024804 |
| 34126 | 6:89007570 | 34128 | 6:89048060 | 34273 | 6:91629835 | 34306 | 6:92470502 | 34307 | 6:92504637 |
| 34410 | 6:94755352 | 34412 | 6:94776615 | 34452 | 6:96090013 | 34453 | 6:96097237 | 34478 | 6:96544331 |
| 34596 | 6:98671599 | 34598 | 6:98696365 | 34635 | 6:99753620 | 34640 | 6:99898377 | 34644 | 6:100026934 |
| 34702 | 6:101156203 | 34718 | 6:101492004 | 34738 | 6:101904289 | 34742 | 6:101951985 | 34771 | 6:102466503 |
| 34774 | 6:102525990 | 34781 | 6:102622674 | 34936 | 6:105596054 | 35002 | 6:107330690 | 35294 | 6:113769965 |
| 35299 | 6:113826996 | 35354 | 6:114785574 | 35370 | 6:115107065 | 35390 | 6:115648265 | 35391 | 6:115670310 |
| 35405 | 6:115976356 | 35426 | 6:116325904 | 35456 | 6:117124190 | 35476 | 7:210754 | 35491 | 7:521674 |
| 35511 | 7:987326 | 35515 | 7:1068152 | 35516 | 7:1068170 | 35638 | 7:3108200 | 35680 | 7:3821837 |
| 35729 | 7:5027580 | 35741 | 7:5125016 | 35859 | 7:6346703 | 35871 | 7:6483779 | 36139 | 7:13283360 |
| 36142 | 7:13315974 | 36174 | 7:13946048 | 36270 | 7:15409865 | 36316 | 7:16530067 | 36366 | 7:17643702 |
| 36378 | 7:17996900 | 36554 | 7:21064478 | 36624 | 7:22566377 | 36628 | 7:22594727 | 36656 | 7:23523707 |
| 36685 | 7:24072015 | 36686 | 7:24142269 | 36697 | 7:24434170 | 36698 | 7:24436989 | 36816 | 7:26920631 |
| 36863 | 7:27929188 | 36943 | 7:29802791 | 36954 | 7:30036916 | 36957 | 7:30105269 | 37015 | 7:31343335 |
| 37063 | 7:32305922 | 37066 | 7:32334690 | 37099 | 7:33229945 | 37111 | 7:33525039 | 37122 | 7:33761341 |
| 37149 | 7:34411840 | 37156 | 7:34553590 | 37176 | 7:35028517 | 37430 | 7:40687768 | 37528 | 7:43355224 |
| 37570 | 7:44672168 | 37748 | 7:48982387 | 37783 | 7:50794543 | 37816 | 7:52176483 | 37833 | 7:52224828 |
| 37838 | 7:52249785 | 38015 | 7:56078682 | 38041 | 7:56774182 | 38048 | 7:56997351 | 38071 | 7:57843022 |
| 38075 | 7:57875535 | 38208 | 7:60668104 | 38263 | 7:61497597 | 38359 | 7:63355823 | 38427 | 7:65211026 |
| 38428 | 7:65229182 | 38429 | 7:65236569 | 38430 | 7:65246212 | 38431 | 7:65252605 | 38470 | 7:66072980 |
| 38560 | 7:67858059 | 38734 | 7:72087070 | 38741 | 7:72254021 | 38780 | 7:73605674 | 38781 | 7:73607632 |
| 38782 | 7:73637074 | 38785 | 7:73667360 | 38867 | 7:75759697 | 38875 | 7:75832195 | 38885 | 7:76174417 |
| 38922 | 7:77054436 | 38942 | 7:77465055 | 38993 | 7:78445222 | 38999 | 7:78709632 | 39099 | 7:80473773 |
| 39124 | 7:80863489 | 39132 | 7:81093522 | 39153 | 7:81316371 | 39179 | 7:81968933 | 39185 | 7:82214691 |
| 39191 | 7:82275842 | 39194 | 7:82369878 | 39298 | 7:85032366 | 39351 | 7:86627973 | 39378 | 7:87376691 |
| 39402 | 7:88102064 | 39416 | 7:88541691 | 39453 | 7:89335556 | 39457 | 7:89404622 | 39489 | 7:90218611 |
| 39512 | 7:90622467 | 39514 | 7:90688919 | 39518 | 7:90701213 | 39520 | 7:90747139 | 39521 | 7:90755504 |
| 39522 | 7:90756120 | 39526 | 7:90805595 | 39527 | 7:90807032 | 39528 | 7:90807192 | 39529 | 7:90807464 |
| 39530 | 7:90807690 | 39531 | 7:90807800 | 39532 | 7:90807968 | 39533 | 7:90808002 | 39534 | 7:90808188 |
| 39535 | 7:90808858 | 39536 | 7:90813061 | 39537 | 7:90813398 | 39539 | 7:90818194 | 39540 | 7:90818916 |
| 39541 | 7:90819613 | 39542 | 7:90820830 | 39543 | 7:90821093 | 39544 | 7:90821542 | 39545 | 7:90821986 |
| 39546 | 7:90822179 | 39547 | 7:90822452 | 39548 | 7:90822601 | 39549 | 7:90823320 | 39550 | 7:90824804 |
| 39551 | 7:90825013 | 39552 | 7:90825646 | 39553 | 7:90825779 | 39554 | 7:90825887 | 39555 | 7:90827178 |
| 39557 | 7:90831014 | 39562 | 7:90842614 | 39567 | 7:90851664 | 39571 | 7:90889577 | 39597 | 7:91489258 |
| 39602 | 7:91611232 | 39603 | 7:91625156 | 39604 | 7:91625242 | 39605 | 7:91625323 | 39606 | 7:91625348 |
| 39609 | 7:91710576 | 39613 | 7:91786432 | 39615 | 7:91796723 | 39616 | 7:91818525 | 39624 | 7:91975067 |
| 39702 | 7:94438004 | 39707 | 7:94529183 | 39744 | 7:95454269 | 39752 | 7:95607084 | 39792 | 7:96165472 |
| 39942 | 7:96409276 | 39956 | 7:96720179 | 39962 | 7:96807889 | 39981 | 7:97434118 | 39990 | 7:97603739 |
| 40255 | 7:104401635 | 40270 | 7:104812484 | 40304 | 7:105679720 | 40323 | 7:106127282 | 40332 | 7:106311771 |
| 40335 | 7:106392644 | 40416 | 7:108497744 | 40421 | 7:108592553 | 40429 | 7:108781177 | 40437 | 7:108939690 |
| 40523 | 8:905854 | 40531 | 8:972589 | 40654 | 8:3953653 | 40665 | 8:4218936 | 40702 | 8:5114851 |
| 40704 | 8:5164202 | 40769 | 8:6831018 | 41003 | 8:12159615 | 41129 | 8:15805194 | 41141 | 8:16218371 |
| 41181 | 8:17034771 | 41209 | 8:17591195 | 41218 | 8:17834202 | 41244 | 8:18677218 | 41417 | 8:23907022 |
| 41564 | 8:25653708 | 41594 | 8:26211730 | 41629 | 8:27285102 | 41727 | 8:29969758 | 41835 | 8:33203967 |
| 41881 | 8:34438846 | 41903 | 8:35034999 | 41929 | 8:35568258 | 41956 | 8:36096463 | 41973 | 8:36469080 |
| 41974 | 8:36489911 | 42016 | 8:37579398 | 42038 | 8:38343034 | 42071 | 8:39659087 | 42104 | 8:40219724 |
| 42149 | 8:41244408 | 42172 | 8:41710832 | 42173 | 8:41735284 | 42187 | 8:42123936 | 42190 | 8:42204721 |
| 42208 | 8:42686594 | 42212 | 8:42720374 | 42216 | 8:42880518 | 42241 | 8:43615936 | 42271 | 8:44484817 |
| 42312 | 8:45608581 | 42314 | 8:45672590 | 42329 | 8:45953160 | 42330 | 8:45985343 | 42332 | 8:46013064 |
| 42395 | 8:47420937 | 42418 | 8:47998648 | 42466 | 8:49248066 | 42519 | 8:50846856 | 42524 | 8:50995576 |
| 42565 | 8:52018600 | 42588 | 8:52629220 | 42591 | 8:52674907 | 42603 | 8:52986604 | 42661 | 8:54554681 |
| 42683 | 8:55077468 | 42694 | 8:55493961 | 42765 | 8:57313074 | 42849 | 8:59626198 | 42853 | 8:59747078 |
| 42916 | 8:61134883 | 42918 | 8:61212042 | 42919 | 8:61235958 | 42922 | 8:61366316 | 42925 | 8:61452677 |
| 43033 | 8:64222797 | 43049 | 8:64876145 | 43067 | 8:65260788 | 43080 | 8:65506614 | 43087 | 8:65773835 |
| 43124 | 8:66668352 | 43200 | 8:68399674 | 43235 | 8:69120902 | 43284 | 8:70429214 | 43306 | 8:70893052 |
| 43311 | 8:70996588 | 43380 | 8:72930313 | 43454 | 8:74772105 | 43457 | 8:74801687 | 43527 | 8:76729847 |
| 43563 | 8:77557071 | 43702 | 8:81553047 | 43722 | 8:82063613 | 43764 | 8:82948559 | 43831 | 8:84602590 |
| 43865 | 8:85540348 | 43883 | 8:85967740 | 43942 | 8:87290730 | 43979 | 8:88449390 | 43993 | 8:88666966 |
| 44077 | 8:90752165 | 44176 | 8:93011122 | 44205 | 8:93906534 | 44262 | 8:95928376 | 44268 | 8:96045608 |
| 44269 | 8:96053216 | 44574 | 8:102774503 | 44648 | 8:104180690 | 44652 | 8:104213734 | 44749 | 8:106594158 |
| 44887 | 8:110915706 | 44899 | 8:111410824 | 45035 | 9:2664478 | 45036 | 9:2667437 | 45037 | 9:2675248 |
| 45038 | 9:2678406 | 45039 | 9:2679109 | 45040 | 9:2681750 | 45041 | 9:2685486 | 45079 | 9:3897414 |
| 45192 | 9:7600098 | 45344 | 9:10670291 | 45354 | 9:10721590 | 45379 | 9:10940149 | 45383 | 9:11053216 |
| 45423 | 9:12009299 | 45432 | 9:12198197 | 45496 | 9:13287682 | 45535 | 9:14277874 | 45589 | 9:15430684 |
| 45660 | 9:16629763 | 45662 | 9:16687402 | 45664 | 9:16727064 | 45676 | 9:17031856 | 45707 | 9:18068635 |
| 45744 | 9:19093497 | 45768 | 9:19999771 | 45832 | 9:21796887 | 45872 | 9:22692551 | 45957 | 9:24735769 |
| 45958 | 9:24739623 | 45959 | 9:24743503 | 45960 | 9:24744612 | 45961 | 9:24746634 | 45962 | 9:24747990 |
| 45963 | 9:24753059 | 45964 | 9:24757919 | 45965 | 9:24760506 | 45991 | 9:25685943 | 46046 | 9:27095343 |
| 46063 | 9:27473901 | 46065 | 9:27477523 | 46066 | 9:27485623 | 46191 | 9:29796827 | 46248 | 9:31114198 |
| 46259 | 9:31366005 | 46302 | 9:32301933 | 46386 | 9:34555386 | 46398 | 9:34906969 | 46462 | 9:36491907 |
| 46486 | 9:37002303 | 46488 | 9:37019786 | 46516 | 9:37778578 | 46518 | 9:37786775 | 46528 | 9:37990154 |
| 46609 | 9:39610171 | 46636 | 9:39977921 | 46841 | 9:45495778 | 46843 | 9:45524861 | 46886 | 9:46918750 |
| 46890 | 9:47015135 | 46938 | 9:48713344 | 46957 | 9:49281683 | 46958 | 9:49310703 | 46959 | 9:49332688 |
| 46969 | 9:49534025 | 46970 | 9:49560954 | 47019 | 9:50797460 | 47025 | 9:51023481 | 47032 | 9:51178639 |
| 47062 | 9:52118530 | 47097 | 9:52875308 | 47136 | 9:54016591 | 47139 | 9:54071333 | 47141 | 9:54121979 |
| 47143 | 9:54193058 | 47144 | 9:54241895 | 47147 | 9:54281836 | 47156 | 9:54592540 | 47165 | 9:54750120 |
| 47200 | 9:55772680 | 47203 | 9:55920760 | 47212 | 9:56203305 | 47237 | 9:56787230 | 47337 | 9:59589683 |
| 47486 | 9:63468566 | 47687 | 9:67996881 | 47691 | 9:68095521 | 47755 | 9:69703203 | 47782 | 9:70360272 |
| 47785 | 9:70387952 | 47920 | 9:73701336 | 48263 | 9:81076190 | 48268 | 9:81121582 | 48490 | 9:85784712 |
| 48495 | 9:85842378 | 48503 | 9:86006398 | 48511 | 9:86271158 | 48513 | 9:86326905 | 48558 | 9:87361936 |
| 48562 | 9:87394371 | 48620 | 9:88414100 | 48662 | 9:89255756 | 48663 | 9:89261575 | 48664 | 9:89266339 |
| 48691 | 9:89514561 | 48721 | 9:90252969 | 48732 | 9:90513278 | 48805 | 9:92056243 | 48810 | 9:92215180 |
| 48813 | 9:92280929 | 48924 | 9:94493145 | 49009 | 9:96178394 | 49069 | 9:96875482 | 49070 | 9:96875798 |
| 49073 | 9:96892725 | 49074 | 9:96894181 | 49096 | 9:97251687 | 49102 | 9:97327317 | 49126 | 9:97749602 |
| 49382 | 9:102242225 | 49467 | 9:103771813 | 49725 | 10:4803921 | 49789 | 10:5830788 | 49811 | 10:6389416 |
| 49838 | 10:6942796 | 49855 | 10:7506260 | 49870 | 10:7723009 | 49879 | 10:7896282 | 49918 | 10:8581573 |
| 49929 | 10:8932852 | 49954 | 10:9647790 | 49959 | 10:9706130 | 50090 | 10:12326160 | 50252 | 10:15438770 |
| 50302 | 10:16346333 | 50344 | 10:17006334 | 50394 | 10:18062302 | 50406 | 10:18136100 | 50425 | 10:18596826 |
| 50457 | 10:19155115 | 50490 | 10:19862667 | 50499 | 10:19959026 | 50578 | 10:21363314 | 50627 | 10:22355891 |
| 50628 | 10:22382830 | 50704 | 10:26294073 | 50713 | 10:26447113 | 50738 | 10:26790460 | 50793 | 10:27960775 |
| 50811 | 10:28566378 | 50812 | 10:28588817 | 50859 | 10:29085444 | 50868 | 10:29217264 | 50934 | 10:31208483 |
| 50952 | 10:31609187 | 50976 | 10:32324758 | 51059 | 10:34358150 | 51094 | 10:34985737 | 51138 | 10:35989426 |
| 51141 | 10:36035947 | 51147 | 10:36261135 | 51164 | 10:36819725 | 51193 | 10:37476784 | 51242 | 10:38651279 |
| 51294 | 10:40046559 | 51405 | 10:43227119 | 51426 | 10:43996215 | 51527 | 10:46095471 | 51600 | 10:47638179 |
| 51609 | 10:47789244 | 51610 | 10:47842702 | 51626 | 10:48320530 | 51629 | 10:48352886 | 51645 | 10:48589436 |
| 51649 | 10:48670398 | 51770 | 10:51376594 | 51773 | 10:51492328 | 51780 | 10:51664061 | 51801 | 10:52103291 |
| 51809 | 10:52286224 | 51824 | 10:52716176 | 51839 | 10:52867771 | 51896 | 10:54578642 | 51972 | 10:56658480 |
| 52166 | 10:62136409 | 52202 | 10:62942087 | 52272 | 10:64614710 | 52278 | 10:64660791 | 52409 | 10:67916863 |
| 52410 | 10:67917609 | 52411 | 10:67918635 | 52412 | 10:67919519 | 52416 | 10:68020797 | 52439 | 10:68405934 |
| 52471 | 10:69067745 | 52500 | 10:69834381 | 52520 | 10:70404549 | 52616 | 10:73493859 | 52730 | 10:76467129 |
| 52964 | 10:81617198 | 53012 | 10:82478545 | 53038 | 10:83193828 | 53039 | 10:83219578 | 53072 | 10:83953443 |
| 53105 | 10:84613620 | 53132 | 10:85160503 | 53157 | 10:85605717 | 53228 | 10:87305772 | 53332 | 10:89626819 |
| 53351 | 10:89936666 | 53372 | 10:90371369 | 53373 | 10:90396997 | 53380 | 10:90447669 | 53400 | 10:90743393 |
| 53411 | 10:90874981 | 53416 | 10:91003179 | 53424 | 10:91160509 | 53468 | 10:92328861 | 53547 | 10:94201919 |
| 53622 | 10:95898502 | 53688 | 10:97342537 | 53729 | 10:98302579 | 53731 | 10:98340013 | 53748 | 10:98561555 |
| 53769 | 10:99070199 | 53816 | 10:100101830 | 53837 | 10:100508994 | 53879 | 10:101228895 | 53880 | 10:101250880 |
| 53936 | 10:102180727 | 53960 | 10:102751503 | 54049 | 11:1244479 | 54111 | 11:1989902 | 54215 | 11:3345485 |
| 54221 | 11:3483025 | 54298 | 11:5089726 | 54369 | 11:6729148 | 54415 | 11:7795378 | 54432 | 11:8194895 |
| 54454 | 11:9031832 | 54487 | 11:9455596 | 54526 | 11:10334522 | 54533 | 11:10452880 | 54563 | 11:11221986 |
| 54593 | 11:11811943 | 54600 | 11:12001967 | 54611 | 11:12371073 | 54687 | 11:13859460 | 54718 | 11:15010708 |
| 54778 | 11:16210947 | 54799 | 11:16834406 | 54848 | 11:18344213 | 54849 | 11:18370765 | 54850 | 11:18396456 |
| 54959 | 11:21470638 | 55054 | 11:23592030 | 55056 | 11:23656903 | 55060 | 11:23740579 | 55083 | 11:24318038 |
| 55092 | 11:24369984 | 55093 | 11:24377983 | 55094 | 11:24393545 | 55096 | 11:24408850 | 55133 | 11:25341825 |
| 55231 | 11:27746425 | 55274 | 11:28736945 | 55276 | 11:28761952 | 55291 | 11:29024122 | 55369 | 11:30763811 |
| 55384 | 11:31041776 | 55392 | 11:31275179 | 55413 | 11:31882807 | 55416 | 11:31964009 | 55428 | 11:32373197 |
| 55474 | 11:33523211 | 55483 | 11:33718317 | 55559 | 11:36181120 | 55589 | 11:37017087 | 55594 | 11:37101530 |
| 55604 | 11:37283583 | 55776 | 11:41848496 | 55903 | 11:45028224 | 55916 | 11:45290515 | 55917 | 11:45313347 |
| 56006 | 11:47096563 | 56017 | 11:47326731 | 56096 | 11:48675652 | 56133 | 11:49165587 | 56145 | 11:49409414 |
| 56148 | 11:49467653 | 56177 | 11:50202868 | 56186 | 11:50607517 | 56242 | 11:52695227 | 56259 | 11:53270728 |
| 56262 | 11:53413882 | 56431 | 11:57988232 | 56468 | 11:58918358 | 56472 | 11:58971050 | 56480 | 11:59181535 |
| 56524 | 11:60468952 | 56540 | 11:60494769 | 56542 | 11:60517676 | 56544 | 11:60567347 | 56558 | 11:60775322 |
| 56559 | 11:60864552 | 56560 | 11:60906972 | 56594 | 11:61768776 | 56624 | 11:62610532 | 56627 | 11:62639185 |
| 56629 | 11:62703391 | 56635 | 11:62811507 | 56646 | 11:63105834 | 56648 | 11:63156170 | 56688 | 11:63881213 |
| 56694 | 11:63980697 | 56771 | 11:66096418 | 56774 | 11:66141532 | 56792 | 11:66500339 | 56805 | 11:66814255 |
| 56807 | 11:66889704 | 56808 | 11:66971573 | 56813 | 11:67084838 | 56826 | 11:67371438 | 56831 | 11:67445332 |
| 56833 | 11:67469876 | 56877 | 11:67856557 | 56883 | 11:68062927 | 56892 | 11:68380416 | 56894 | 11:68423346 |
| 56911 | 11:68884476 | 56915 | 11:68962608 | 56951 | 11:69554436 | 56976 | 11:70005013 | 56985 | 11:70216257 |
| 56988 | 11:70246998 | 56993 | 11:70300762 | 56996 | 11:70378153 | 57042 | 11:70810858 | 57106 | 11:72079051 |
| 57111 | 11:72169040 | 57219 | 11:74557125 | 57222 | 11:74693404 | 57231 | 11:75039779 | 57238 | 11:75245474 |
| 57239 | 11:75278281 | 57240 | 11:75301517 | 57244 | 11:75330473 | 57261 | 11:75610538 | 57269 | 11:75826371 |
| 57278 | 11:76006290 | 57282 | 11:76108140 | 57286 | 11:76291884 | 57299 | 11:76699098 | 57313 | 11:77009264 |
| 57332 | 11:77262392 | 57343 | 11:77494347 | 57355 | 11:77817486 | 57389 | 11:78010446 | 57391 | 11:78051004 |
| 57414 | 11:78619581 | 57476 | 11:79716380 | 57485 | 11:79945955 | 57486 | 11:80005355 | 57490 | 11:80160161 |
| 57509 | 11:80535106 | 57524 | 11:80835889 | 57534 | 11:81336077 | 57547 | 11:81697133 | 57619 | 11:82922365 |
| 57628 | 11:83118146 | 57667 | 11:84097016 | 57691 | 11:84931500 | 57733 | 11:85771451 | 57735 | 11:85810220 |
| 57756 | 11:86216179 | 57813 | 11:87691339 | 57824 | 11:87901219 | 57825 | 11:87933139 | 57838 | 11:87991576 |
| 57856 | 11:88008802 | 57871 | 11:88092604 | 57875 | 11:88114495 | 57883 | 11:88435971 | 57905 | 11:88999569 |
| 57918 | 11:89363137 | 58014 | 11:91483828 | 58015 | 11:91524490 | 58040 | 11:92016623 | 58199 | 11:95837864 |
| 58223 | 11:96479338 | 58254 | 11:97291509 | 58262 | 11:97559369 | 58265 | 11:97669268 | 58346 | 11:99292200 |
| 58369 | 11:99693004 | 58374 | 11:99779968 | 58381 | 11:99910936 | 58425 | 11:100730926 | 58480 | 11:101720696 |
| 58706 | 11:105729430 | 58710 | 11:105964095 | 58711 | 11:106026712 | 58887 | 12:3816364 | 58893 | 12:4017888 |
| 58924 | 12:4735467 | 59120 | 12:10585260 | 59121 | 12:10614578 | 59249 | 12:13606719 | 59254 | 12:13766685 |
| 59263 | 12:13857247 | 59320 | 12:15004714 | 59351 | 12:15565363 | 59376 | 12:15986894 | 59402 | 12:16562247 |
| 59446 | 12:17721084 | 59635 | 12:22358320 | 59648 | 12:22684761 | 59650 | 12:22780186 | 59656 | 12:22878113 |
| 59657 | 12:22955187 | 59658 | 12:22957971 | 59662 | 12:23024428 | 59686 | 12:23553969 | 59699 | 12:23968918 |
| 59725 | 12:24562389 | 59751 | 12:25043912 | 59818 | 12:26941585 | 59863 | 12:28021962 | 59864 | 12:28031415 |
| 59901 | 12:29149304 | 59931 | 12:30073919 | 59940 | 12:30237550 | 59970 | 12:30965466 | 59975 | 12:30995990 |
| 60105 | 12:34339978 | 60136 | 12:35202030 | 60146 | 12:35329106 | 60185 | 12:36506500 | 60202 | 12:37059151 |
| 60204 | 12:37117228 | 60206 | 12:37121439 | 60213 | 12:37336023 | 60264 | 12:39602051 | 60268 | 12:39726857 |
| 60368 | 12:43543060 | 60451 | 12:46609222 | 60452 | 12:46632594 | 60607 | 12:50328249 | 60608 | 12:50367458 |
| 60610 | 12:50405882 | 60655 | 12:51430846 | 60683 | 12:52226211 | 60688 | 12:52270911 | 60735 | 12:53308347 |
| 60768 | 12:53915464 | 60769 | 12:53924609 | 60780 | 12:54114793 | 60908 | 12:57839576 | 60967 | 12:59688270 |
| 61136 | 12:65124144 | 61209 | 12:67262559 | 61285 | 12:68831445 | 61316 | 12:69427741 | 61337 | 12:69681738 |
| 61357 | 12:70744278 | 61358 | 12:70747193 | 61411 | 12:72948314 | 61418 | 12:73037670 | 61419 | 12:73063509 |
| 61433 | 12:73378861 | 61437 | 12:73452468 | 61748 | 12:80191366 | 61809 | 12:81602505 | 61930 | 12:84189393 |
| 62050 | 12:86604613 | 62158 | 13:1818757 | 62173 | 13:2108292 | 62212 | 13:2694888 | 62217 | 13:2721397 |
| 62219 | 13:2775064 | 62220 | 13:2800282 | 62221 | 13:2813363 | 62257 | 13:3832665 | 62281 | 13:4414592 |
| 62310 | 13:5228105 | 62405 | 13:7418765 | 62519 | 13:10200560 | 62521 | 13:10297467 | 62539 | 13:10605573 |
| 62667 | 13:14736984 | 62723 | 13:16382635 | 62724 | 13:16385352 | 62728 | 13:16481332 | 62729 | 13:16481881 |
| 62730 | 13:16482962 | 62731 | 13:16484064 | 62732 | 13:16486013 | 62733 | 13:16487474 | 62734 | 13:16489553 |
| 62735 | 13:16494712 | 62736 | 13:16495172 | 62737 | 13:16495924 | 62738 | 13:16496828 | 62739 | 13:16497723 |
| 62740 | 13:16498500 | 62741 | 13:16499450 | 62742 | 13:16500106 | 62743 | 13:16505307 | 62774 | 13:17297996 |
| 62785 | 13:17376202 | 62832 | 13:18167455 | 62850 | 13:18708210 | 62851 | 13:18716183 | 62853 | 13:18721208 |
| 62854 | 13:18721251 | 62855 | 13:18723232 | 62856 | 13:18723968 | 62857 | 13:18724391 | 62858 | 13:18724682 |
| 62859 | 13:18724926 | 62860 | 13:18725319 | 62861 | 13:18725595 | 62862 | 13:18725670 | 62863 | 13:18725747 |
| 62864 | 13:18726353 | 62865 | 13:18727332 | 62866 | 13:18727382 | 62867 | 13:18728046 | 62868 | 13:18728216 |
| 62869 | 13:18729395 | 62870 | 13:18729428 | 62871 | 13:18729514 | 62872 | 13:18729597 | 62873 | 13:18732582 |
| 62874 | 13:18733271 | 62875 | 13:18733719 | 62876 | 13:18733742 | 62877 | 13:18733760 | 62878 | 13:18733884 |
| 62879 | 13:18735443 | 62882 | 13:18791943 | 62931 | 13:19690250 | 62932 | 13:19710676 | 62940 | 13:19814205 |
| 62956 | 13:20094078 | 62957 | 13:20180098 | 62960 | 13:20267627 | 62965 | 13:20376037 | 62975 | 13:20455892 |
| 62984 | 13:20631768 | 63067 | 13:22364650 | 63079 | 13:22622712 | 63125 | 13:24378395 | 63206 | 13:26030778 |
| 63286 | 13:28051297 | 63307 | 13:28631602 | 63318 | 13:28789636 | 63452 | 13:31297457 | 63463 | 13:31535167 |
| 63484 | 13:32188070 | 63485 | 13:32192701 | 63486 | 13:32195543 | 63488 | 13:32202239 | 63489 | 13:32206561 |
| 63490 | 13:32214896 | 63498 | 13:32363871 | 63515 | 13:32606184 | 63519 | 13:32649822 | 63534 | 13:32908367 |
| 63643 | 13:35552308 | 63655 | 13:35893139 | 63853 | 13:40115588 | 63937 | 13:42022321 | 63954 | 13:42455044 |
| 63957 | 13:42527459 | 63962 | 13:42563978 | 63996 | 13:42702842 | 64016 | 13:43224017 | 64031 | 13:43618688 |
| 64043 | 13:43868655 | 64084 | 13:44564660 | 64090 | 13:44735441 | 64093 | 13:44846012 | 64094 | 13:44877072 |
| 64095 | 13:44877728 | 64096 | 13:44883203 | 64097 | 13:44884376 | 64098 | 13:44891644 | 64099 | 13:44900803 |
| 64100 | 13:44901430 | 64101 | 13:44902570 | 64102 | 13:44908683 | 64104 | 13:44929091 | 64106 | 13:44941064 |
| 64107 | 13:44945347 | 64108 | 13:44954742 | 64109 | 13:44957480 | 64110 | 13:44959530 | 64111 | 13:44971418 |
| 64113 | 13:44974314 | 64114 | 13:44977218 | 64118 | 13:44984974 | 64120 | 13:44991424 | 64123 | 13:44994329 |
| 64124 | 13:44996032 | 64161 | 13:45580238 | 64220 | 13:46808920 | 64238 | 13:47284141 | 64266 | 13:47867949 |
| 64336 | 13:49568372 | 64337 | 13:49569350 | 64338 | 13:49570779 | 64339 | 13:49571658 | 64416 | 13:52005450 |
| 64426 | 13:52366895 | 64431 | 13:52518024 | 64456 | 13:52984857 | 64458 | 13:53006663 | 64466 | 13:53163449 |
| 64512 | 13:54065681 | 64629 | 13:57014498 | 64667 | 13:57988686 | 64672 | 13:58092691 | 64887 | 13:62737371 |
| 64900 | 13:62849693 | 64901 | 13:62871349 | 64910 | 13:63113668 | 64979 | 13:64765574 | 64992 | 13:65197131 |
| 65003 | 13:65421742 | 65046 | 13:66169238 | 65096 | 13:67321578 | 65143 | 13:68639312 | 65145 | 13:68664064 |
| 65159 | 13:69019407 | 65312 | 13:72652771 | 65332 | 13:73024654 | 65430 | 13:74713520 | 65439 | 13:75021624 |
| 65447 | 13:75318964 | 65448 | 13:75354084 | 65503 | 13:76376258 | 65507 | 13:76438492 | 65517 | 13:76641970 |
| 65529 | 13:76798256 | 65764 | 13:81126899 | 65905 | 14:1006940 | 65963 | 14:2212373 | 65993 | 14:3074708 |
| 65995 | 14:3098041 | 66091 | 14:4323988 | 66140 | 14:5386856 | 66177 | 14:5966351 | 66207 | 14:6303201 |
| 66289 | 14:7818027 | 66305 | 14:8003665 | 66326 | 14:8349092 | 66332 | 14:8441986 | 66350 | 14:8630299 |
| 66373 | 14:8996524 | 66493 | 14:11272085 | 66494 | 14:11275856 | 66495 | 14:11277881 | 66496 | 14:11278042 |
| 66497 | 14:11281126 | 66499 | 14:11294774 | 66500 | 14:11299050 | 66547 | 14:12222307 | 66586 | 14:12811226 |
| 66627 | 14:14043986 | 66687 | 14:15518676 | 66690 | 14:15530827 | 66691 | 14:15559743 | 66692 | 14:15601008 |
| 66693 | 14:15625272 | 66694 | 14:15626105 | 66695 | 14:15631968 | 66696 | 14:15643325 | 66698 | 14:15672366 |
| 66717 | 14:16009930 | 66718 | 14:16010978 | 66719 | 14:16022809 | 66724 | 14:16161546 | 66750 | 14:16670887 |
| 66775 | 14:17233811 | 66795 | 14:17760515 | 66799 | 14:17770830 | 66831 | 14:18480356 | 66832 | 14:18510376 |
| 66909 | 14:20574088 | 66969 | 14:21764414 | 66976 | 14:21914329 | 66987 | 14:22199112 | 66989 | 14:22291692 |
| 66991 | 14:22353534 | 66994 | 14:22402037 | 66997 | 14:22459009 | 66998 | 14:22460066 | 66999 | 14:22492158 |
| 67000 | 14:22499302 | 67001 | 14:22499882 | 67002 | 14:22500823 | 67003 | 14:22500981 | 67004 | 14:22501165 |
| 67005 | 14:22502609 | 67006 | 14:22503786 | 67007 | 14:22505668 | 67009 | 14:22551848 | 67010 | 14:22559672 |
| 67011 | 14:22559976 | 67012 | 14:22560498 | 67013 | 14:22560939 | 67014 | 14:22561597 | 67015 | 14:22562389 |
| 67016 | 14:22562808 | 67017 | 14:22562827 | 67018 | 14:22563098 | 67019 | 14:22563125 | 67020 | 14:22563136 |
| 67021 | 14:22563534 | 67022 | 14:22564168 | 67023 | 14:22564379 | 67024 | 14:22564874 | 67025 | 14:22565102 |
| 67026 | 14:22565724 | 67027 | 14:22565752 | 67028 | 14:22565896 | 67029 | 14:22565903 | 67030 | 14:22566273 |
| 67031 | 14:22566433 | 67032 | 14:22566621 | 67033 | 14:22567218 | 67034 | 14:22567252 | 67035 | 14:22567396 |
| 67036 | 14:22567418 | 67037 | 14:22567497 | 67038 | 14:22567570 | 67039 | 14:22568006 | 67040 | 14:22568446 |
| 67041 | 14:22568460 | 67042 | 14:22568517 | 67043 | 14:22568557 | 67044 | 14:22568700 | 67045 | 14:22568919 |
| 67046 | 14:22569314 | 67047 | 14:22569464 | 67048 | 14:22569577 | 67049 | 14:22569581 | 67050 | 14:22569980 |
| 67051 | 14:22570044 | 67052 | 14:22570058 | 67053 | 14:22570080 | 67054 | 14:22570301 | 67055 | 14:22570448 |
| 67056 | 14:22570466 | 67057 | 14:22570726 | 67058 | 14:22571422 | 67059 | 14:22573154 | 67060 | 14:22574799 |
| 67061 | 14:22579399 | 67062 | 14:22579809 | 67063 | 14:22581401 | 67064 | 14:22584062 | 67065 | 14:22584452 |
| 67066 | 14:22592312 | 67071 | 14:22671158 | 67072 | 14:22680795 | 67073 | 14:22681826 | 67079 | 14:22781305 |
| 67080 | 14:22830132 | 67081 | 14:22839488 | 67082 | 14:22841040 | 67083 | 14:22842180 | 67084 | 14:22861656 |
| 67085 | 14:22861757 | 67086 | 14:22861840 | 67087 | 14:22866278 | 67088 | 14:22867321 | 67089 | 14:22870443 |
| 67090 | 14:22872231 | 67091 | 14:22872707 | 67092 | 14:22875389 | 67094 | 14:22883080 | 67096 | 14:22887406 |
| 67097 | 14:22888105 | 67098 | 14:22895284 | 67099 | 14:22896212 | 67100 | 14:22899088 | 67101 | 14:22900130 |
| 67102 | 14:22900140 | 67103 | 14:22903945 | 67104 | 14:22904357 | 67105 | 14:22905670 | 67106 | 14:22915081 |
| 67107 | 14:22915304 | 67108 | 14:22916046 | 67109 | 14:22916365 | 67110 | 14:22917130 | 67111 | 14:22917282 |
| 67112 | 14:22918679 | 67113 | 14:22919099 | 67114 | 14:22922271 | 67115 | 14:22925305 | 67116 | 14:22926221 |
| 67117 | 14:22933624 | 67118 | 14:22935277 | 67119 | 14:22940329 | 67120 | 14:22941327 | 67121 | 14:22944551 |
| 67122 | 14:22945851 | 67123 | 14:22950339 | 67124 | 14:22951594 | 67125 | 14:22952187 | 67126 | 14:22952264 |
| 67127 | 14:22958318 | 67128 | 14:22959278 | 67129 | 14:22962984 | 67130 | 14:22963530 | 67131 | 14:22963552 |
| 67132 | 14:22963956 | 67133 | 14:22964530 | 67134 | 14:22968925 | 67135 | 14:22969225 | 67136 | 14:22969659 |
| 67137 | 14:22970008 | 67138 | 14:22983250 | 67139 | 14:22983836 | 67140 | 14:22986080 | 67141 | 14:22989108 |
| 67142 | 14:22989880 | 67143 | 14:22989917 | 67144 | 14:22995750 | 67145 | 14:22995861 | 67146 | 14:22999212 |
| 67147 | 14:22999475 | 67148 | 14:23000859 | 67149 | 14:23001109 | 67150 | 14:23001803 | 67151 | 14:23004072 |
| 67152 | 14:23005237 | 67153 | 14:23009266 | 67157 | 14:23168386 | 67175 | 14:23216693 | 67180 | 14:23217714 |
| 67181 | 14:23217718 | 67183 | 14:23219140 | 67189 | 14:23221714 | 67191 | 14:23232498 | 67193 | 14:23241336 |
| 67194 | 14:23244133 | 67195 | 14:23245996 | 67196 | 14:23246004 | 67197 | 14:23246030 | 67198 | 14:23246116 |
| 67199 | 14:23246121 | 67200 | 14:23246668 | 67201 | 14:23247018 | 67202 | 14:23247588 | 67203 | 14:23247661 |
| 67204 | 14:23247870 | 67205 | 14:23247878 | 67206 | 14:23247920 | 67207 | 14:23247940 | 67208 | 14:23248135 |
| 67209 | 14:23248201 | 67210 | 14:23248211 | 67211 | 14:23248448 | 67212 | 14:23248692 | 67213 | 14:23248770 |
| 67214 | 14:23248888 | 67215 | 14:23248943 | 67216 | 14:23249702 | 67217 | 14:23249738 | 67218 | 14:23249950 |
| 67219 | 14:23249976 | 67220 | 14:23250082 | 67221 | 14:23250315 | 67222 | 14:23250686 | 67223 | 14:23250690 |
| 67224 | 14:23250828 | 67225 | 14:23250871 | 67226 | 14:23250914 | 67227 | 14:23251020 | 67228 | 14:23251302 |
| 67229 | 14:23251581 | 67230 | 14:23251684 | 67231 | 14:23252354 | 67232 | 14:23252429 | 67233 | 14:23252563 |
| 67234 | 14:23252757 | 67235 | 14:23252809 | 67236 | 14:23253043 | 67237 | 14:23253210 | 67238 | 14:23253295 |
| 67239 | 14:23253300 | 67240 | 14:23253568 | 67241 | 14:23253746 | 67242 | 14:23253786 | 67243 | 14:23253805 |
| 67244 | 14:23254426 | 67245 | 14:23254453 | 67246 | 14:23254563 | 67247 | 14:23254639 | 67248 | 14:23254769 |
| 67249 | 14:23254807 | 67250 | 14:23254919 | 67251 | 14:23255007 | 67252 | 14:23255159 | 67253 | 14:23255587 |
| 67254 | 14:23255824 | 67255 | 14:23256710 | 67256 | 14:23257050 | 67257 | 14:23257092 | 67258 | 14:23257183 |
| 67259 | 14:23257229 | 67260 | 14:23257379 | 67261 | 14:23257441 | 67262 | 14:23257644 | 67263 | 14:23257665 |
| 67264 | 14:23257775 | 67265 | 14:23258452 | 67266 | 14:23258542 | 67267 | 14:23262536 | 67268 | 14:23262544 |
| 67269 | 14:23262984 | 67270 | 14:23264575 | 67271 | 14:23264736 | 67272 | 14:23265786 | 67273 | 14:23265991 |
| 67274 | 14:23266384 | 67275 | 14:23271813 | 67276 | 14:23273276 | 67277 | 14:23273801 | 67278 | 14:23274957 |
| 67279 | 14:23277049 | 67280 | 14:23278232 | 67281 | 14:23278652 | 67282 | 14:23278927 | 67283 | 14:23279593 |
| 67284 | 14:23279712 | 67285 | 14:23285130 | 67286 | 14:23288477 | 67287 | 14:23288921 | 67288 | 14:23289158 |
| 67289 | 14:23289589 | 67290 | 14:23289947 | 67291 | 14:23292612 | 67293 | 14:23296927 | 67294 | 14:23297204 |
| 67295 | 14:23298062 | 67296 | 14:23300304 | 67297 | 14:23314460 | 67298 | 14:23314730 | 67299 | 14:23314761 |
| 67300 | 14:23321577 | 67301 | 14:23326588 | 67302 | 14:23338890 | 67303 | 14:23343150 | 67304 | 14:23346065 |
| 67306 | 14:23383008 | 67308 | 14:23392938 | 67309 | 14:23408928 | 67310 | 14:23409204 | 67311 | 14:23409494 |
| 67312 | 14:23410644 | 67313 | 14:23410867 | 67314 | 14:23411712 | 67315 | 14:23414348 | 67316 | 14:23414443 |
| 67317 | 14:23421256 | 67318 | 14:23421807 | 67319 | 14:23422909 | 67320 | 14:23423768 | 67321 | 14:23423823 |
| 67322 | 14:23424726 | 67323 | 14:23426056 | 67324 | 14:23427548 | 67325 | 14:23429529 | 67326 | 14:23430628 |
| 67327 | 14:23430999 | 67328 | 14:23431167 | 67329 | 14:23438214 | 67330 | 14:23439941 | 67331 | 14:23440995 |
| 67332 | 14:23441483 | 67333 | 14:23443135 | 67334 | 14:23444334 | 67335 | 14:23446328 | 67336 | 14:23447775 |
| 67337 | 14:23450091 | 67338 | 14:23456836 | 67339 | 14:23458201 | 67340 | 14:23459603 | 67343 | 14:23528515 |
| 67344 | 14:23532117 | 67354 | 14:23600267 | 67355 | 14:23607944 | 67356 | 14:23622754 | 67357 | 14:23630896 |
| 67358 | 14:23639458 | 67359 | 14:23652804 | 67368 | 14:23825669 | 67370 | 14:23829057 | 67371 | 14:23829066 |
| 67372 | 14:23829235 | 67373 | 14:23830314 | 67375 | 14:23831223 | 67376 | 14:23831351 | 67377 | 14:23831451 |
| 67378 | 14:23831720 | 67379 | 14:23832062 | 67380 | 14:23832448 | 67381 | 14:23832632 | 67382 | 14:23832846 |
| 67383 | 14:23832941 | 67384 | 14:23833173 | 67385 | 14:23833835 | 67386 | 14:23834262 | 67387 | 14:23834735 |
| 67388 | 14:23834925 | 67389 | 14:23835136 | 67390 | 14:23835627 | 67391 | 14:23835870 | 67392 | 14:23835971 |
| 67393 | 14:23836131 | 67394 | 14:23836398 | 67395 | 14:23836443 | 67396 | 14:23836603 | 67397 | 14:23836691 |
| 67398 | 14:23836914 | 67399 | 14:23837002 | 67400 | 14:23838557 | 67401 | 14:23839177 | 67402 | 14:23840827 |
| 67403 | 14:23842863 | 67404 | 14:23848780 | 67405 | 14:23848797 | 67406 | 14:23848827 | 67407 | 14:23849503 |
| 67408 | 14:23852492 | 67409 | 14:23852815 | 67410 | 14:23852921 | 67411 | 14:23853184 | 67412 | 14:23853223 |
| 67413 | 14:23854487 | 67414 | 14:23856672 | 67415 | 14:23860303 | 67416 | 14:23862417 | 67418 | 14:23868149 |
| 67419 | 14:23882209 | 67420 | 14:23887365 | 67421 | 14:23887370 | 67422 | 14:23937950 | 67423 | 14:23945118 |
| 67425 | 14:23992830 | 67426 | 14:24019648 | 67430 | 14:24107226 | 67436 | 14:24297582 | 67440 | 14:24350585 |
| 67446 | 14:24477168 | 67447 | 14:24512384 | 67449 | 14:24516122 | 67450 | 14:24519148 | 67451 | 14:24519442 |
| 67452 | 14:24520182 | 67453 | 14:24522732 | 67454 | 14:24524138 | 67455 | 14:24527219 | 67456 | 14:24528754 |
| 67457 | 14:24532678 | 67458 | 14:24535392 | 67459 | 14:24536887 | 67460 | 14:24537213 | 67461 | 14:24537249 |
| 67462 | 14:24537652 | 67463 | 14:24537937 | 67464 | 14:24538192 | 67465 | 14:24538201 | 67466 | 14:24538525 |
| 67467 | 14:24538984 | 67468 | 14:24539180 | 67469 | 14:24539228 | 67470 | 14:24539315 | 67471 | 14:24539324 |
| 67472 | 14:24539556 | 67473 | 14:24539584 | 67474 | 14:24539696 | 67475 | 14:24540007 | 67476 | 14:24540057 |
| 67477 | 14:24540244 | 67478 | 14:24540270 | 67479 | 14:24542751 | 67481 | 14:24571024 | 67482 | 14:24580144 |
| 67483 | 14:24587368 | 67484 | 14:24594780 | 67485 | 14:24604764 | 67486 | 14:24609404 | 67487 | 14:24618912 |
| 67488 | 14:24644457 | 67493 | 14:24767026 | 67495 | 14:24790463 | 67496 | 14:24825146 | 67497 | 14:24859655 |
| 67500 | 14:24940746 | 67501 | 14:24941221 | 67502 | 14:24967239 | 67503 | 14:24967988 | 67504 | 14:24973247 |
| 67505 | 14:24978104 | 67506 | 14:24979482 | 67507 | 14:24982302 | 67508 | 14:24983741 | 67509 | 14:24986291 |
| 67510 | 14:25002052 | 67511 | 14:25030583 | 67513 | 14:25082860 | 67516 | 14:25152867 | 67517 | 14:25158355 |
| 67526 | 14:25266063 | 67530 | 14:25440652 | 67533 | 14:25548588 | 67534 | 14:25548611 | 67535 | 14:25548858 |
| 67536 | 14:25549944 | 67537 | 14:25550026 | 67538 | 14:25550835 | 67539 | 14:25550879 | 67540 | 14:25551206 |
| 67541 | 14:25551423 | 67542 | 14:25551661 | 67543 | 14:25551868 | 67544 | 14:25552256 | 67545 | 14:25552486 |
| 67546 | 14:25552782 | 67547 | 14:25552984 | 67548 | 14:25553034 | 67549 | 14:25553066 | 67550 | 14:25553217 |
| 67552 | 14:25555831 | 67613 | 14:26283998 | 67621 | 14:26472341 | 67627 | 14:26644064 | 67628 | 14:26666519 |
| 67686 | 14:28503857 | 67690 | 14:28611968 | 67696 | 14:28706164 | 67714 | 14:29246244 | 67717 | 14:29321450 |
| 67723 | 14:29455092 | 67743 | 14:29818291 | 67756 | 14:30089537 | 67838 | 14:32001174 | 67864 | 14:32341495 |
| 67882 | 14:32807225 | 67883 | 14:32884654 | 67887 | 14:32989933 | 67910 | 14:33408077 | 67918 | 14:33505061 |
| 67920 | 14:33551756 | 67973 | 14:34950627 | 67979 | 14:35239973 | 67980 | 14:35240016 | 67981 | 14:35240021 |
| 67982 | 14:35240029 | 67983 | 14:35240427 | 67984 | 14:35240523 | 67985 | 14:35240587 | 67986 | 14:35240759 |
| 67987 | 14:35240947 | 67988 | 14:35241115 | 67989 | 14:35241328 | 67990 | 14:35241474 | 67991 | 14:35241672 |
| 67992 | 14:35265340 | 67998 | 14:35425257 | 68003 | 14:35595531 | 68006 | 14:35751572 | 68010 | 14:35894072 |
| 68024 | 14:36171691 | 68025 | 14:36225604 | 68027 | 14:36267667 | 68045 | 14:36581802 | 68070 | 14:37486005 |
| 68085 | 14:37852302 | 68092 | 14:38020858 | 68098 | 14:38241778 | 68104 | 14:38511542 | 68107 | 14:38549650 |
| 68121 | 14:38865578 | 68136 | 14:39381019 | 68138 | 14:39446327 | 68154 | 14:39861013 | 68181 | 14:40565849 |
| 68184 | 14:40702265 | 68188 | 14:40809782 | 68195 | 14:41149021 | 68202 | 14:41340996 | 68219 | 14:41739836 |
| 68239 | 14:42191184 | 68242 | 14:42286395 | 68265 | 14:42846993 | 68302 | 14:44079419 | 68338 | 14:44933472 |
| 68350 | 14:45112392 | 68358 | 14:45283009 | 68368 | 14:45610432 | 68374 | 14:45681264 | 68376 | 14:45772293 |
| 68391 | 14:46030748 | 68402 | 14:46236143 | 68403 | 14:46236637 | 68404 | 14:46237110 | 68405 | 14:46238761 |
| 68406 | 14:46241868 | 68407 | 14:46244088 | 68408 | 14:46250918 | 68418 | 14:46418593 | 68424 | 14:46603107 |
| 68441 | 14:46995812 | 68444 | 14:47059409 | 68474 | 14:48033192 | 68483 | 14:48202902 | 68512 | 14:48835114 |
| 68819 | 14:56925358 | 68973 | 14:60573159 | 68974 | 14:60591022 | 69042 | 14:62002265 | 69055 | 14:62159723 |
| 69056 | 14:62160097 | 69093 | 14:62960532 | 69145 | 14:64219535 | 69158 | 14:64538337 | 69236 | 14:66565905 |
| 69296 | 14:67751963 | 69405 | 14:70557126 | 69479 | 14:72621724 | 69527 | 14:73767156 | 69546 | 14:74251050 |
| 69666 | 14:77010786 | 69676 | 14:77348388 | 69701 | 14:77989698 | 69824 | 14:80695704 | 69841 | 14:81015684 |
| 70102 | 15:4692832 | 70287 | 15:5550484 | 70295 | 15:5724091 | 70403 | 15:8239471 | 70559 | 15:13400749 |
| 70578 | 15:14002939 | 70584 | 15:14158577 | 70619 | 15:14996109 | 70634 | 15:15286305 | 70711 | 15:16549253 |
| 70719 | 15:16763241 | 70746 | 15:17193061 | 70781 | 15:18160360 | 70783 | 15:18238844 | 70920 | 15:21418237 |
| 70933 | 15:21792771 | 70955 | 15:22295223 | 71033 | 15:23794449 | 71075 | 15:24300165 | 71076 | 15:24314377 |
| 71084 | 15:24406959 | 71092 | 15:24625723 | 71106 | 15:24813538 | 71225 | 15:27497541 | 71246 | 15:28011102 |
| 71266 | 15:28279946 | 71274 | 15:28453282 | 71275 | 15:28497953 | 71313 | 15:28951920 | 71373 | 15:30506918 |
| 71421 | 15:31626657 | 71443 | 15:32064162 | 71459 | 15:32448502 | 71467 | 15:32684505 | 71489 | 15:33066384 |
| 71494 | 15:33140066 | 71500 | 15:33283643 | 71516 | 15:33757310 | 71519 | 15:33784264 | 71542 | 15:34144843 |
| 71544 | 15:34205212 | 71548 | 15:34249244 | 71550 | 15:34273013 | 71565 | 15:34545054 | 71594 | 15:35124174 |
| 71637 | 15:36171696 | 71643 | 15:36281231 | 71644 | 15:36316689 | 71668 | 15:36835278 | 71670 | 15:36913421 |
| 71721 | 15:38013431 | 71735 | 15:38345972 | 71814 | 15:40225597 | 71892 | 15:41883212 | 71918 | 15:42285508 |
| 71919 | 15:42310362 | 71922 | 15:42461633 | 71961 | 15:43061218 | 71966 | 15:43113772 | 71983 | 15:43322372 |
| 72013 | 15:43808690 | 72034 | 15:44213989 | 72038 | 15:44321644 | 72039 | 15:44348210 | 72041 | 15:44377304 |
| 72043 | 15:44404296 | 72081 | 15:45199864 | 72107 | 15:45767348 | 72150 | 15:46515608 | 72163 | 15:46717734 |
| 72166 | 15:46782723 | 72172 | 15:46863275 | 72181 | 15:47142381 | 72226 | 15:48040954 | 72245 | 15:48275779 |
| 72278 | 15:49421011 | 72280 | 15:49469161 | 72294 | 15:49994423 | 72324 | 15:51010384 | 72368 | 15:51564179 |
| 72387 | 15:51844292 | 72392 | 15:51981969 | 72395 | 15:51992306 | 72396 | 15:52000306 | 72403 | 15:52160095 |
| 72434 | 15:52969357 | 72473 | 15:53341502 | 72559 | 15:55149996 | 72592 | 15:56124060 | 72635 | 15:56767431 |
| 72636 | 15:56774921 | 72637 | 15:56780183 | 72638 | 15:56782530 | 72639 | 15:56783614 | 72640 | 15:56784736 |
| 72641 | 15:56785971 | 72642 | 15:56797332 | 72643 | 15:56807110 | 72646 | 15:56815479 | 72647 | 15:56818346 |
| 72648 | 15:56829119 | 72649 | 15:56831561 | 72650 | 15:56832894 | 72651 | 15:56833644 | 72652 | 15:56834347 |
| 72653 | 15:56835434 | 72654 | 15:56836055 | 72655 | 15:56836949 | 72656 | 15:56838230 | 72657 | 15:56838871 |
| 72658 | 15:56840204 | 72659 | 15:56841525 | 72660 | 15:56842815 | 72661 | 15:56843668 | 72662 | 15:56845155 |
| 72663 | 15:56851874 | 72664 | 15:56853748 | 72665 | 15:56855128 | 72666 | 15:56856657 | 72667 | 15:56868653 |
| 72668 | 15:56869977 | 72690 | 15:57484879 | 72695 | 15:57536250 | 72767 | 15:58882110 | 72769 | 15:58979088 |
| 72784 | 15:59472006 | 72833 | 15:60847989 | 72869 | 15:61639615 | 72884 | 15:61810673 | 72900 | 15:62235431 |
| 72906 | 15:62456943 | 72909 | 15:62484563 | 72910 | 15:62511268 | 72918 | 15:62618258 | 72922 | 15:62694367 |
| 72976 | 15:63833091 | 72988 | 15:64092090 | 73022 | 15:64696473 | 73048 | 15:65009502 | 73052 | 15:65067229 |
| 73130 | 15:66545124 | 73132 | 15:66614036 | 73460 | 15:75001650 | 73465 | 15:75074384 | 73560 | 15:77284842 |
| 73564 | 15:77342823 | 73599 | 15:77992295 | 73602 | 15:78001169 | 73610 | 15:78017138 | 73626 | 15:78135787 |
| 73719 | 15:80696386 | 73731 | 15:81021484 | 73760 | 15:81377169 | 73768 | 15:81639702 | 73794 | 15:82390046 |
| 73813 | 15:82751747 | 73814 | 15:82766106 | 73826 | 15:82951372 | 73834 | 15:83031817 | 73835 | 15:83043883 |
| 73836 | 15:83046338 | 73837 | 15:83054032 | 73838 | 15:83058015 | 73839 | 15:83063624 | 73945 | 16:1306469 |
| 73949 | 16:1350556 | 73955 | 16:1432322 | 74009 | 16:2521425 | 74012 | 16:2584565 | 74027 | 16:3043683 |
| 74058 | 16:3453206 | 74076 | 16:3954831 | 74079 | 16:3986796 | 74218 | 16:7674017 | 74248 | 16:8845857 |
| 74278 | 16:9938750 | 74286 | 16:10168137 | 74356 | 16:12347316 | 74366 | 16:12489625 | 74382 | 16:12870890 |
| 74389 | 16:12974308 | 74390 | 16:12974917 | 74391 | 16:12975122 | 74392 | 16:12975220 | 74393 | 16:12975427 |
| 74394 | 16:12975489 | 74395 | 16:12975573 | 74396 | 16:12975644 | 74460 | 16:14726498 | 74462 | 16:14841844 |
| 74520 | 16:16433701 | 74547 | 16:17397768 | 74579 | 16:18551432 | 74583 | 16:18633047 | 74592 | 16:18835344 |
| 74621 | 16:19568001 | 74641 | 16:20043212 | 74669 | 16:20811063 | 74702 | 16:21584184 | 74853 | 16:25049887 |
| 74963 | 16:26927766 | 74986 | 16:27682201 | 75041 | 16:29174251 | 75060 | 16:29760720 | 75062 | 16:29811081 |
| 75073 | 16:30136403 | 75075 | 16:30161226 | 75076 | 16:30185523 | 75134 | 16:31704359 | 75143 | 16:31896105 |
| 75150 | 16:32035645 | 75163 | 16:32472255 | 75166 | 16:32570779 | 75268 | 16:35045999 | 75271 | 16:35171790 |
| 75403 | 16:38151804 | 75444 | 16:39447477 | 75514 | 16:41852637 | 75544 | 16:41964938 | 75601 | 16:44301023 |
| 75602 | 16:44309062 | 75603 | 16:44341029 | 75675 | 16:46932908 | 75678 | 16:47002315 | 75805 | 16:49746045 |
| 75821 | 16:50190328 | 75822 | 16:50240719 | 75839 | 16:50745473 | 75941 | 16:53280547 | 75975 | 16:54055090 |
| 75976 | 16:54077469 | 75987 | 16:54386293 | 76037 | 16:55777077 | 76379 | 16:63448062 | 76420 | 16:64372019 |
| 76474 | 16:65163914 | 76705 | 16:70129856 | 76767 | 16:71527211 | 76903 | 16:74041271 | 76913 | 16:74276107 |
| 76922 | 16:74520515 | 76932 | 16:74777059 | 76992 | 16:75746134 | 77018 | 16:76272938 | 77019 | 16:76273016 |
| 77054 | 16:77113967 | 77055 | 16:77114869 | 77056 | 16:77115406 | 77057 | 16:77115489 | 77058 | 16:77115511 |
| 77059 | 16:77115516 | 77060 | 16:77117453 | 77061 | 16:77124198 | 77227 | 16:80002018 | 77342 | 17:2538151 |
| 77417 | 17:4163909 | 77468 | 17:5077558 | 77480 | 17:5375823 | 77508 | 17:5999177 | 77613 | 17:8012177 |
| 77632 | 17:8453908 | 77860 | 17:14044398 | 77915 | 17:14966600 | 78029 | 17:17475831 | 78037 | 17:17737256 |
| 78038 | 17:17740826 | 78097 | 17:19033632 | 78335 | 17:26269335 | 78410 | 17:28015501 | 78497 | 17:30487913 |
| 78560 | 17:31986086 | 78574 | 17:32403842 | 78580 | 17:32489547 | 78638 | 17:33714773 | 78647 | 17:33972684 |
| 78649 | 17:34035563 | 78655 | 17:34228544 | 78680 | 17:34921588 | 78708 | 17:35827456 | 78896 | 17:40827993 |
| 79097 | 17:45794152 | 79104 | 17:45901126 | 79186 | 17:47856402 | 79401 | 17:52535678 | 79405 | 17:52588133 |
| 79527 | 17:55767778 | 79528 | 17:55769246 | 79534 | 17:55892881 | 79577 | 17:57167393 | 79582 | 17:57279391 |
| 79748 | 17:60357076 | 79754 | 17:60395994 | 79778 | 17:60781502 | 79835 | 17:61664121 | 79837 | 17:61736121 |
| 79860 | 17:62385783 | 79871 | 17:62699773 | 79873 | 17:62731132 | 79876 | 17:62794522 | 79877 | 17:62820495 |
| 79916 | 17:63612425 | 79928 | 17:63638557 | 79962 | 17:64318575 | 80008 | 17:65293792 | 80030 | 17:65746912 |
| 80050 | 17:66155014 | 80061 | 17:66243332 | 80076 | 17:66473136 | 80079 | 17:66581559 | 80083 | 17:66695254 |
| 80109 | 17:67239268 | 80249 | 17:70356568 | 80285 | 17:70987613 | 80303 | 17:71333851 | 80363 | 17:72400135 |
| 80592 | 18:3769740 | 80636 | 18:4682446 | 80731 | 18:6411919 | 80773 | 18:7301014 | 80800 | 18:7879780 |
| 80807 | 18:8137616 | 80811 | 18:8163590 | 80812 | 18:8166965 | 80813 | 18:8169505 | 80848 | 18:8910517 |
| 80881 | 18:9393342 | 80887 | 18:9441291 | 80933 | 18:10341158 | 80973 | 18:11082461 | 81013 | 18:11950846 |
| 81055 | 18:13195401 | 81339 | 18:19844264 | 81450 | 18:22200958 | 81455 | 18:22265614 | 81495 | 18:23178646 |
| 81501 | 18:23288724 | 81645 | 18:26295597 | 81707 | 18:27873728 | 81901 | 18:33007379 | 81923 | 18:33448672 |
| 81932 | 18:33625498 | 81933 | 18:33654608 | 81936 | 18:33679712 | 81937 | 18:33689480 | 81939 | 18:33731026 |
| 81940 | 18:33765822 | 81945 | 18:33836053 | 81946 | 18:33868226 | 82061 | 18:36768945 | 82065 | 18:36877351 |
| 82083 | 18:37328492 | 82099 | 18:37566458 | 82114 | 18:37873077 | 82177 | 18:39306524 | 82178 | 18:39344592 |
| 82179 | 18:39385561 | 82181 | 18:39440689 | 82184 | 18:39467461 | 82186 | 18:39488321 | 82190 | 18:39592965 |
| 82370 | 18:43157279 | 82379 | 18:43316323 | 82382 | 18:43368967 | 82508 | 18:46424023 | 82580 | 18:46784427 |
| 82583 | 18:46865748 | 82670 | 18:48351248 | 82698 | 18:49004338 | 82732 | 18:49949153 | 82736 | 18:49996660 |
| 82831 | 18:52094939 | 82855 | 18:52727408 | 82885 | 18:53596284 | 82887 | 18:53619711 | 82909 | 18:53938427 |
| 82919 | 18:54093736 | 82940 | 18:54546398 | 82942 | 18:54586408 | 82983 | 18:55141854 | 82984 | 18:55173246 |
| 83013 | 18:55944026 | 83110 | 18:57018026 | 83153 | 18:57798250 | 83172 | 18:57998293 | 83257 | 18:59897080 |
| 83291 | 18:61008032 | 83319 | 18:61724391 | 83595 | 19:414615 | 84130 | 19:13223983 | 84131 | 19:13225470 |
| 84175 | 19:13777565 | 84315 | 19:16698614 | 84335 | 19:16990785 | 84387 | 19:17914841 | 84393 | 19:18036338 |
| 84409 | 19:18449930 | 84457 | 19:19539554 | 84577 | 19:21388744 | 84702 | 19:23904908 | 84703 | 19:23933777 |
| 84742 | 19:24383348 | 84781 | 19:24846129 | 84846 | 19:25706233 | 85007 | 19:28109982 | 85011 | 19:28147842 |
| 85061 | 19:29186854 | 85119 | 19:30424898 | 85120 | 19:30433795 | 85121 | 19:30435544 | 85178 | 19:31844296 |
| 85211 | 19:32510963 | 85345 | 19:34890238 | 85357 | 19:34981210 | 85366 | 19:35152019 | 85409 | 19:36144917 |
| 85481 | 19:37101024 | 85602 | 19:40338519 | 85855 | 19:45407828 | 85921 | 19:46516531 | 85941 | 19:47101703 |
| 85951 | 19:47151917 | 85971 | 19:47631810 | 86015 | 19:48407091 | 86087 | 19:49926869 | 86124 | 19:50694652 |
| 86143 | 19:50789408 | 86202 | 19:51325894 | 86259 | 19:52650212 | 86283 | 19:53097970 | 86292 | 19:53198253 |
| 86293 | 19:53206247 | 86319 | 19:53762930 | 86348 | 19:54137367 | 86384 | 19:54758347 | 86416 | 19:55474861 |
| 86488 | 19:56006886 | 86522 | 19:56138971 | 86531 | 19:56253321 | 86568 | 19:56726108 | 86580 | 19:56980273 |
| 86813 | 19:61050416 | 86822 | 19:61175458 | 86893 | 19:62160575 | 86898 | 19:62249612 | 86900 | 19:62272214 |
| 86997 | 20:909954 | 87043 | 20:1738655 | 87052 | 20:1886807 | 87083 | 20:2362867 | 87101 | 20:2682912 |
| 87142 | 20:3442790 | 87149 | 20:3585646 | 87174 | 20:4263007 | 87176 | 20:4284310 | 87177 | 20:4331799 |
| 87190 | 20:4516902 | 87193 | 20:4587717 | 87194 | 20:4587833 | 87195 | 20:4587839 | 87196 | 20:4588506 |
| 87197 | 20:4588549 | 87198 | 20:4588618 | 87199 | 20:4588637 | 87200 | 20:4588654 | 87203 | 20:4620871 |
| 87204 | 20:4621075 | 87205 | 20:4621110 | 87206 | 20:4621122 | 87207 | 20:4622072 | 87208 | 20:4622166 |
| 87209 | 20:4622378 | 87210 | 20:4622780 | 87211 | 20:4622804 | 87212 | 20:4622902 | 87213 | 20:4623044 |
| 87214 | 20:4623114 | 87215 | 20:4623572 | 87216 | 20:4624342 | 87217 | 20:4624492 | 87218 | 20:4624645 |
| 87219 | 20:4625064 | 87220 | 20:4625438 | 87221 | 20:4626150 | 87222 | 20:4626194 | 87223 | 20:4626939 |
| 87224 | 20:4627062 | 87225 | 20:4628179 | 87226 | 20:4628239 | 87227 | 20:4629196 | 87228 | 20:4629784 |
| 87229 | 20:4630247 | 87230 | 20:4630301 | 87231 | 20:4630500 | 87232 | 20:4630563 | 87233 | 20:4630761 |
| 87234 | 20:4630879 | 87235 | 20:4630925 | 87236 | 20:4631015 | 87237 | 20:4631031 | 87238 | 20:4631157 |
| 87239 | 20:4631655 | 87240 | 20:4632251 | 87241 | 20:4632285 | 87242 | 20:4632908 | 87243 | 20:4633050 |
| 87244 | 20:4633087 | 87245 | 20:4633727 | 87246 | 20:4633738 | 87247 | 20:4633830 | 87248 | 20:4634166 |
| 87249 | 20:4634320 | 87250 | 20:4634619 | 87251 | 20:4634657 | 87252 | 20:4635386 | 87253 | 20:4635483 |
| 87254 | 20:4635990 | 87255 | 20:4637075 | 87256 | 20:4637125 | 87257 | 20:4638775 | 87258 | 20:4638910 |
| 87259 | 20:4639096 | 87260 | 20:4640118 | 87261 | 20:4640234 | 87262 | 20:4644059 | 87263 | 20:4644327 |
| 87264 | 20:4652157 | 87265 | 20:4654656 | 87267 | 20:4658089 | 87268 | 20:4658953 | 87269 | 20:4661324 |
| 87270 | 20:4665088 | 87271 | 20:4666775 | 87272 | 20:4667789 | 87275 | 20:4685771 | 87277 | 20:4690335 |
| 87278 | 20:4690908 | 87279 | 20:4691249 | 87280 | 20:4697066 | 87281 | 20:4697631 | 87282 | 20:4701728 |
| 87283 | 20:4701832 | 87284 | 20:4708012 | 87285 | 20:4709731 | 87286 | 20:4710087 | 87287 | 20:4712587 |
| 87288 | 20:4713160 | 87289 | 20:4715176 | 87290 | 20:4716043 | 87291 | 20:4726582 | 87292 | 20:4728514 |
| 87293 | 20:4730765 | 87294 | 20:4732330 | 87296 | 20:4736819 | 87297 | 20:4741050 | 87298 | 20:4750279 |
| 87299 | 20:4751162 | 87300 | 20:4752621 | 87301 | 20:4753160 | 87302 | 20:4757934 | 87303 | 20:4758722 |
| 87304 | 20:4761361 | 87305 | 20:4763955 | 87306 | 20:4770162 | 87309 | 20:4797785 | 87310 | 20:4805282 |
| 87311 | 20:4812157 | 87312 | 20:4819449 | 87314 | 20:4838678 | 87315 | 20:4841899 | 87316 | 20:4842019 |
| 87317 | 20:4846348 | 87318 | 20:4850816 | 87319 | 20:4851612 | 87320 | 20:4853215 | 87321 | 20:4854589 |
| 87322 | 20:4863651 | 87323 | 20:4873619 | 87324 | 20:4873645 | 87325 | 20:4873658 | 87326 | 20:4873667 |
| 87327 | 20:4880129 | 87328 | 20:4885163 | 87329 | 20:4922714 | 87330 | 20:4927232 | 87332 | 20:4928741 |
| 87333 | 20:4936772 | 87334 | 20:4937498 | 87335 | 20:4938011 | 87336 | 20:4938521 | 87337 | 20:4938780 |
| 87338 | 20:4938885 | 87339 | 20:4939294 | 87340 | 20:4940782 | 87341 | 20:4940805 | 87342 | 20:4944507 |
| 87343 | 20:4947601 | 87344 | 20:4949204 | 87345 | 20:4964757 | 87346 | 20:4965179 | 87347 | 20:4965760 |
| 87348 | 20:4969010 | 87349 | 20:4971347 | 87350 | 20:4971831 | 87351 | 20:4972329 | 87352 | 20:4974179 |
| 87354 | 20:4975337 | 87355 | 20:4978069 | 87356 | 20:4978213 | 87357 | 20:4978475 | 87359 | 20:4982228 |
| 87360 | 20:4984846 | 87361 | 20:4985523 | 87362 | 20:4986398 | 87363 | 20:4988142 | 87364 | 20:4990340 |
| 87365 | 20:4991225 | 87367 | 20:4992704 | 87368 | 20:4992712 | 87369 | 20:4993558 | 87370 | 20:4993669 |
| 87371 | 20:4993957 | 87373 | 20:4999299 | 87374 | 20:4999988 | 87376 | 20:5002715 | 87377 | 20:5003850 |
| 87378 | 20:5004638 | 87379 | 20:5008738 | 87380 | 20:5009598 | 87383 | 20:5040971 | 87385 | 20:5045805 |
| 87386 | 20:5049196 | 87387 | 20:5054905 | 87388 | 20:5055091 | 87389 | 20:5055857 | 87390 | 20:5058209 |
| 87391 | 20:5070761 | 87392 | 20:5074751 | 87393 | 20:5075106 | 87394 | 20:5076823 | 87395 | 20:5079687 |
| 87397 | 20:5081368 | 87399 | 20:5082509 | 87400 | 20:5107041 | 87402 | 20:5129728 | 87415 | 20:5282517 |
| 87429 | 20:5485968 | 87434 | 20:5633232 | 87435 | 20:5665364 | 87436 | 20:5695030 | 87438 | 20:5754805 |
| 87439 | 20:5796361 | 87449 | 20:6060819 | 87450 | 20:6062848 | 87451 | 20:6063007 | 87452 | 20:6063251 |
| 87453 | 20:6063553 | 87454 | 20:6063862 | 87455 | 20:6063984 | 87456 | 20:6064358 | 87457 | 20:6064442 |
| 87458 | 20:6066220 | 87459 | 20:6066792 | 87460 | 20:6066880 | 87461 | 20:6067197 | 87462 | 20:6069267 |
| 87463 | 20:6069326 | 87464 | 20:6069531 | 87465 | 20:6072588 | 87466 | 20:6073779 | 87467 | 20:6076915 |
| 87470 | 20:6090071 | 87477 | 20:6208301 | 87484 | 20:6325657 | 87488 | 20:6444472 | 87490 | 20:6526713 |
| 87509 | 20:6914755 | 87513 | 20:7017878 | 87516 | 20:7049088 | 87519 | 20:7079494 | 87521 | 20:7089351 |
| 87537 | 20:7437979 | 87539 | 20:7473329 | 87548 | 20:7706288 | 87550 | 20:7739561 | 87557 | 20:7958728 |
| 87559 | 20:7978859 | 87564 | 20:8099584 | 87566 | 20:8120294 | 87570 | 20:8190372 | 87573 | 20:8244125 |
| 87599 | 20:8976316 | 87608 | 20:9074647 | 87609 | 20:9107005 | 87662 | 20:10266518 | 87686 | 20:10946439 |
| 87698 | 20:11213830 | 87702 | 20:11274635 | 87711 | 20:11438125 | 87734 | 20:11948110 | 87736 | 20:12001911 |
| 87747 | 20:12228546 | 87750 | 20:12265622 | 87755 | 20:12409400 | 87758 | 20:12445726 | 87824 | 20:13931338 |
| 87826 | 20:13967565 | 87829 | 20:14050805 | 87830 | 20:14120849 | 87885 | 20:16058541 | 87923 | 20:17088438 |
| 87931 | 20:17251657 | 88103 | 20:21950333 | 88188 | 20:22481257 | 88190 | 20:22578241 | 88220 | 20:23180410 |
| 88233 | 20:23492042 | 88239 | 20:23575761 | 88241 | 20:23623587 | 88244 | 20:23695033 | 88334 | 20:25681663 |
| 88342 | 20:26011480 | 88352 | 20:26319951 | 88372 | 20:26721455 | 88379 | 20:26929602 | 88486 | 20:29337360 |
| 88501 | 20:29797692 | 88594 | 20:32271969 | 88629 | 20:33323766 | 88635 | 20:33477656 | 88636 | 20:33482995 |
| 88651 | 20:33788019 | 88843 | 20:38077292 | 88880 | 20:39076620 | 88893 | 20:39383171 | 88900 | 20:39458208 |
| 88901 | 20:39462501 | 88903 | 20:39469722 | 88906 | 20:39485628 | 88970 | 20:41005245 | 88987 | 20:41215061 |
| 89024 | 20:41990636 | 89201 | 20:46912182 | 89292 | 20:49691751 | 89327 | 20:50794831 | 89331 | 20:50908612 |
| 89334 | 20:50953384 | 89388 | 20:52906490 | 89424 | 20:53680094 | 89427 | 20:53779182 | 89490 | 20:55580535 |
| 89491 | 20:55615423 | 89501 | 20:55920256 | 89504 | 20:55944017 | 89603 | 20:58030736 | 89607 | 20:58078386 |
| 89748 | 20:61558977 | 89850 | 20:63825165 | 89852 | 20:63847390 | 89855 | 20:63882214 | 89947 | 20:65988032 |
| 89964 | 20:66468697 | 90010 | 20:67443129 | 90042 | 20:68014966 | 90105 | 20:69097447 | 90110 | 20:69174801 |
| 90114 | 20:69288227 | 90116 | 20:69317277 | 90161 | 20:70296851 | 90314 | 21:2109801 | 90321 | 21:2416354 |
| 90340 | 21:2937636 | 90422 | 21:5168513 | 90441 | 21:5776117 | 90447 | 21:5802937 | 90449 | 21:5865604 |
| 90452 | 21:5932063 | 90531 | 21:8044281 | 90549 | 21:8125084 | 90577 | 21:8739099 | 90673 | 21:10967078 |
| 90676 | 21:11012534 | 90870 | 21:15628004 | 90871 | 21:15629471 | 90872 | 21:15634222 | 90873 | 21:15635039 |
| 90874 | 21:15636617 | 90875 | 21:15640534 | 90876 | 21:15644415 | 90877 | 21:15648341 | 90878 | 21:15652428 |
| 90879 | 21:15656729 | 90964 | 21:17632245 | 90983 | 21:18074742 | 90987 | 21:18139041 | 91008 | 21:18709801 |
| 91009 | 21:18745959 | 91041 | 21:19240261 | 91119 | 21:20568083 | 91133 | 21:20667605 | 91163 | 21:21254778 |
| 91164 | 21:21278489 | 91244 | 21:22980503 | 91310 | 21:24894924 | 91377 | 21:26762300 | 91417 | 21:27993786 |
| 91435 | 21:28714736 | 91624 | 21:33373608 | 91661 | 21:33774652 | 91702 | 21:34416654 | 91768 | 21:35722374 |
| 91868 | 21:38504846 | 91877 | 21:38644972 | 91941 | 21:40541468 | 91985 | 21:41630642 | 92011 | 21:42193358 |
| 92020 | 21:42461759 | 92029 | 21:42678313 | 92115 | 21:44563811 | 92120 | 21:44677322 | 92171 | 21:46302745 |
| 92332 | 21:49387921 | 92335 | 21:49487176 | 92397 | 21:51289914 | 92407 | 21:51606765 | 92484 | 21:53761679 |
| 92488 | 21:53814553 | 92726 | 21:57261794 | 92848 | 21:59894217 | 92855 | 21:59981922 | 92967 | 21:62224560 |
| 93037 | 21:64128429 | 93155 | 21:66126088 | 93201 | 21:67026936 | 93296 | 21:69054783 | 93298 | 21:69111628 |
| 93301 | 21:69165607 | 93601 | 22:5786344 | 93653 | 22:6683071 | 93663 | 22:6874059 | 93668 | 22:6973667 |
| 93683 | 22:7347580 | 93785 | 22:10006376 | 93807 | 22:10285746 | 93819 | 22:10597656 | 93821 | 22:10648705 |
| 93829 | 22:10879669 | 93832 | 22:10961055 | 93894 | 22:12251220 | 94047 | 22:16130993 | 94110 | 22:17624753 |
| 94177 | 22:19212118 | 94255 | 22:21157473 | 94279 | 22:21651340 | 94283 | 22:21690013 | 94326 | 22:22930737 |
| 94380 | 22:24606457 | 94410 | 22:25532201 | 94504 | 22:28088504 | 94526 | 22:28449795 | 94614 | 22:30572708 |
| 94616 | 22:30617079 | 94621 | 22:30762175 | 94689 | 22:32709942 | 94691 | 22:32740432 | 94806 | 22:35660903 |
| 94908 | 22:38005262 | 94968 | 22:39268641 | 95000 | 22:39706378 | 95013 | 22:39906000 | 95037 | 22:40607655 |
| 95117 | 22:42731135 | 95121 | 22:42898400 | 95122 | 22:42936001 | 95284 | 22:46414503 | 95300 | 22:46810720 |
| 95307 | 22:46910055 | 95353 | 22:47726147 | 95583 | 22:53279742 | 95622 | 22:54013319 | 95666 | 22:54873498 |
| 95669 | 22:54947766 | 95676 | 22:55073957 | 95716 | 22:55626326 | 95781 | 22:56400223 | 95784 | 22:56416067 |
| 95785 | 22:56419658 | 95786 | 22:56420213 | 95809 | 22:56829490 | 95908 | 22:58476458 | 95913 | 22:58524949 |
| 95955 | 22:59413554 | 96087 | 23:2001057 | 96090 | 23:2028074 | 96118 | 23:2653886 | 96121 | 23:2672734 |
| 96124 | 23:2696666 | 96171 | 23:3661084 | 96177 | 23:3744075 | 96232 | 23:4789896 | 96422 | 23:8682465 |
| 96482 | 23:10137524 | 96546 | 23:11326784 | 96550 | 23:11418685 | 96552 | 23:11493234 | 96644 | 23:13147408 |
| 96647 | 23:13234890 | 96777 | 23:15576268 | 96872 | 23:17807855 | 96930 | 23:19250498 | 96931 | 23:19271779 |
| 96933 | 23:19307959 | 96941 | 23:19459539 | 96947 | 23:19483200 | 96959 | 23:19635143 | 96965 | 23:19711944 |
| 96996 | 23:20201294 | 97043 | 23:21249255 | 97051 | 23:21366692 | 97071 | 23:21956033 | 97096 | 23:22583782 |
| 97278 | 23:25346058 | 97357 | 23:27009758 | 97451 | 23:27999156 | 97501 | 23:28242781 | 97780 | 23:32969917 |
| 97828 | 23:34109165 | 97943 | 23:37547849 | 97981 | 23:38637301 | 98382 | 23:45418705 | 98389 | 23:45533685 |
| 98404 | 23:46021030 | 98438 | 23:46778964 | 98556 | 23:48912747 | 98631 | 23:50033982 | 98724 | 23:51773706 |
| 98783 | 24:520222 | 98832 | 24:1526726 | 98846 | 24:1793795 | 98892 | 24:2708333 | 98894 | 24:2766149 |
| 98913 | 24:3184889 | 98915 | 24:3208218 | 98935 | 24:3712541 | 98952 | 24:4048939 | 98964 | 24:4188152 |
| 98982 | 24:4612976 | 98994 | 24:5077959 | 99037 | 24:6230000 | 99125 | 24:8320822 | 99297 | 24:10714691 |
| 99353 | 24:12020111 | 99356 | 24:12116867 | 99361 | 24:12381665 | 99369 | 24:12616739 | 99402 | 24:13417942 |
| 99414 | 24:13675366 | 99451 | 24:14795404 | 99594 | 24:19151288 | 99665 | 24:20730048 | 99691 | 24:21214702 |
| 99863 | 24:25023821 | 99870 | 24:25259209 | 99898 | 24:25782545 | 99909 | 24:25992482 | 100009 | 24:28570245 |
| 100053 | 24:29754026 | 100059 | 24:29814575 | 100120 | 24:31415877 | 100199 | 24:32894188 | 100245 | 24:34122422 |
| 100257 | 24:34395443 | 100267 | 24:34719362 | 100285 | 24:35185903 | 100316 | 24:35736460 | 100323 | 24:35860154 |
| 100338 | 24:36195505 | 100358 | 24:37011897 | 100362 | 24:37166678 | 100364 | 24:37203930 | 100429 | 24:39199046 |
| 100782 | 24:46297155 | 100791 | 24:46409035 | 100919 | 24:49840655 | 100982 | 24:51488828 | 101005 | 24:51881818 |
| 101010 | 24:51956661 | 101014 | 24:52037619 | 101245 | 24:57052356 | 101347 | 24:59016719 | 101349 | 24:59106967 |
| 101394 | 24:60056629 | 101395 | 24:60093951 | 101469 | 24:61534805 | 101551 | 25:1163011 | 101605 | 25:1918071 |
| 101614 | 25:2105032 | 101618 | 25:2179579 | 101802 | 25:3997589 | 101849 | 25:4921820 | 101868 | 25:5339582 |
| 101958 | 25:6766372 | 101966 | 25:6952022 | 102005 | 25:7896632 | 102011 | 25:7949848 | 102018 | 25:8118349 |
| 102131 | 25:10832103 | 102149 | 25:11113745 | 102307 | 25:14593246 | 102318 | 25:14917112 | 102323 | 25:15012071 |
| 102329 | 25:15106926 | 102356 | 25:15663604 | 102365 | 25:15912161 | 102380 | 25:16148690 | 102399 | 25:16456322 |
| 102413 | 25:16841496 | 102459 | 25:17915319 | 102460 | 25:17952726 | 102462 | 25:18009869 | 102490 | 25:18825304 |
| 102523 | 25:19553139 | 102596 | 25:21400170 | 102612 | 25:21756847 | 102647 | 25:22463379 | 102656 | 25:22697194 |
| 102676 | 25:23209925 | 102679 | 25:23393931 | 102695 | 25:23751702 | 102713 | 25:24073747 | 102724 | 25:24300333 |
| 102732 | 25:24496560 | 102767 | 25:25119747 | 102774 | 25:25175370 | 102851 | 25:26611224 | 102875 | 25:27289405 |
| 102971 | 25:29170307 | 102973 | 25:29196073 | 102976 | 25:29227741 | 103031 | 25:30198863 | 103036 | 25:30292715 |
| 103045 | 25:30543167 | 103047 | 25:30590381 | 103165 | 25:33056358 | 103226 | 25:34314708 | 103243 | 25:34536825 |
| 103265 | 25:34878010 | 103273 | 25:35037480 | 103294 | 25:35416991 | 103369 | 25:36281889 | 103468 | 25:37785020 |
| 103522 | 25:38925016 | 103562 | 25:39799663 | 103598 | 25:40282487 | 103603 | 25:40362153 | 103698 | 25:42167568 |
| 103761 | 26:1999995 | 103766 | 26:2270743 | 103811 | 26:2874049 | 103964 | 26:7323964 | 104015 | 26:8514472 |
| 104061 | 26:9801561 | 104175 | 26:12542210 | 104214 | 26:13232302 | 104243 | 26:13935413 | 104244 | 26:13974845 |
| 104248 | 26:14058998 | 104265 | 26:14560437 | 104266 | 26:14562388 | 104420 | 26:18423545 | 104469 | 26:19395441 |
| 104485 | 26:19632302 | 104679 | 26:24855737 | 104714 | 26:25459976 | 104715 | 26:25463718 | 104716 | 26:25465826 |
| 104717 | 26:25467173 | 104781 | 26:26561752 | 104860 | 26:28504622 | 104925 | 26:30397842 | 104932 | 26:30643098 |
| 104935 | 26:30753391 | 104947 | 26:31085230 | 105378 | 26:41389219 | 105381 | 26:41451309 | 105394 | 26:41900876 |
| 105445 | 26:42982393 | 105499 | 26:44238502 | 105742 | 26:49454109 | 105754 | 26:49637614 | 105813 | 26:51001537 |
| 105824 | 26:51205579 | 105851 | 26:51911589 | 105854 | 26:51990348 | 105886 | 27:1292211 | 105932 | 27:2099758 |
| 105955 | 27:2545985 | 106036 | 27:4477596 | 106056 | 27:5041534 | 106064 | 27:5224821 | 106086 | 27:5489950 |
| 106115 | 27:6134492 | 106135 | 27:7463182 | 106203 | 27:8803096 | 106277 | 27:10171403 | 106312 | 27:11344637 |
| 106334 | 27:11976000 | 106374 | 27:12998392 | 106452 | 27:13945896 | 106510 | 27:15278766 | 106547 | 27:16013203 |
| 106553 | 27:16091265 | 106558 | 27:16161541 | 106608 | 27:16989882 | 106617 | 27:17229318 | 106655 | 27:18039702 |
| 106668 | 27:18444933 | 106672 | 27:18549440 | 106674 | 27:18561932 | 106695 | 27:18999723 | 106753 | 27:20126660 |
| 106800 | 27:21693284 | 106837 | 27:22606696 | 106839 | 27:22612038 | 106840 | 27:22625012 | 106841 | 27:22635635 |
| 106851 | 27:22872901 | 106892 | 27:23844184 | 106958 | 27:25097722 | 106963 | 27:25227473 | 107031 | 27:26703643 |
| 107032 | 27:26707702 | 107033 | 27:26709108 | 107034 | 27:26717112 | 107035 | 27:26752145 | 107037 | 27:26821155 |
| 107116 | 27:28602334 | 107142 | 27:29184261 | 107215 | 27:30821123 | 107219 | 27:30967912 | 107275 | 27:31920386 |
| 107418 | 27:35019984 | 107424 | 27:35045715 | 107434 | 27:35072059 | 107439 | 27:35098802 | 107452 | 27:35316815 |
| 107478 | 27:35710124 | 107512 | 27:36366267 | 107541 | 27:37168180 | 107629 | 27:38769358 | 107630 | 27:38784049 |
| 107632 | 27:38825952 | 107648 | 27:39079734 | 107650 | 27:39140091 | 107659 | 27:39414442 | 107684 | 27:40138920 |
| 107723 | 27:41177956 | 107855 | 27:44120630 | 107940 | 28:1026816 | 107943 | 28:1063432 | 107978 | 28:1669722 |
| 107979 | 28:1676667 | 107980 | 28:1688640 | 108035 | 28:3310204 | 108064 | 28:4807450 | 108072 | 28:5021515 |
| 108093 | 28:5430772 | 108094 | 28:5471888 | 108109 | 28:5837959 | 108120 | 28:5926093 | 108142 | 28:6534383 |
| 108248 | 28:9052050 | 108503 | 28:15552135 | 108505 | 28:15620196 | 108621 | 28:17961688 | 108634 | 28:18086730 |
| 108720 | 28:20471303 | 108761 | 28:21757902 | 108775 | 28:22091224 | 108882 | 28:24568402 | 108911 | 28:25196334 |
| 108950 | 28:26054541 | 108997 | 28:26764566 | 109005 | 28:26976598 | 109043 | 28:27784145 | 109225 | 28:31516271 |
| 109243 | 28:31960964 | 109334 | 28:33369552 | 109355 | 28:33823084 | 109363 | 28:34002468 | 109397 | 28:34663948 |
| 109420 | 28:34999208 | 109504 | 28:37068693 | 109511 | 28:37195142 | 109515 | 28:37311281 | 109519 | 28:37446774 |
| 109549 | 28:37921033 | 109557 | 28:38098897 | 109621 | 28:39341830 | 109741 | 28:40986070 | 109744 | 28:41014073 |
| 109881 | 28:43434313 | 109929 | 28:44311425 | 109935 | 28:44453574 | 109945 | 28:44780535 | 109958 | 28:44981684 |
| 109988 | 28:45527255 | 110035 | 29:1014606 | 110080 | 29:1997329 | 110082 | 29:2117573 | 110221 | 29:5006464 |
| 110233 | 29:5358686 | 110234 | 29:5358699 | 110248 | 29:5885429 | 110252 | 29:5979872 | 110429 | 29:10496324 |
| 110449 | 29:11034651 | 110590 | 29:14470262 | 110606 | 29:14866248 | 110721 | 29:17901471 | 110722 | 29:17951025 |
| 110723 | 29:17955354 | 110769 | 29:19855058 | 110770 | 29:19861520 | 110771 | 29:19862538 | 110985 | 29:25169363 |
| 110987 | 29:25193462 | 111011 | 29:25759092 | 111063 | 29:27485292 | 111143 | 29:28949555 | 111155 | 29:29347332 |
| 111211 | 29:30136039 | 111212 | 29:30157180 | 111221 | 29:30347114 | 111336 | 29:32940189 | 111340 | 29:33039863 |
| 111384 | 29:34046874 | 111390 | 29:34239637 | 111402 | 29:34523872 | 111422 | 29:34808241 | 111479 | 29:35951280 |
| 111481 | 29:35999607 | 111550 | 29:37304798 | 111551 | 29:37320062 | 111658 | 29:40811556 | 111659 | 29:40812741 |
| 111660 | 29:40813920 | 111661 | 29:40818497 | 111662 | 29:40820427 | 111663 | 29:40820963 | 111664 | 29:40821065 |
| 111666 | 29:40825872 | 111806 | 29:43407527 | 111808 | 29:43424442 | 111892 | 29:44721351 | 111901 | 29:45041058 |
| 111904 | 29:45172298 | 111928 | 29:45712362 | 111979 | 29:46833503 | 111989 | 29:46997866 | 111995 | 29:47125684 |
| 111996 | 29:47150455 | 112100 | 29:49234278 | 112124 | 29:49722188 | 112140 | 29:50217955 | 112141 | 29:50260533 |

;
and
a screening method of the sixth marker panel comprises the following steps: allowing genotyping results of the BovineHD chip of Illumina and the GGP Bovine 100K chip of Neogen in the Huaxi cattle population to have quality control at a minor allele frequency (MAF) of greater than 0.1, a genotype deletion rate of less than 10%, and a P value of Hardy-Weinberg equilibrium (HWE) testing of 1×10⁻⁶, and deleting invalid sites while retaining effective information marker sites in the Huaxi cattle population; and
the sites of the seventh marker panel are shown in the following table:

| NO. | Position | NO. | Position | NO. | Position | NO. | Position | NO. | Position |
|---|---|---|---|---|---|---|---|---|---|
| 1 | 1:198611 | 2 | 1:354105 | 3 | 1:413686 | 4 | 1:503436 | 5 | 1:539006 |
| 6 | 1:561519 | 8 | 1:752778 | 114 | 1:2548682 | 115 | 1:2645413 | 116 | 1:2738165 |
| 120 | 1:2859590 | 248 | 1:5217586 | 249 | 1:5308367 | 394 | 1:8387543 | 518 | 1:11192831 |
| 542 | 1:11918795 | 549 | 1:12187898 | 551 | 1:12295655 | 578 | 1:13034308 | 581 | 1:13135427 |
| 624 | 1:13936005 | 625 | 1:14033343 | 634 | 1:14325639 | 689 | 1:15749427 | 709 | 1:16317871 |
| 793 | 1:19108624 | 795 | 1:19246215 | 816 | 1:19761054 | 817 | 1:19840115 | 906 | 1:22225114 |
| 926 | 1:22716338 | 927 | 1:22813570 | 942 | 1:23156851 | 943 | 1:23255421 | 950 | 1:23587167 |
| 1081 | 1:27110041 | 1096 | 1:27690352 | 1099 | 1:27826641 | 1121 | 1:28708529 | 1184 | 1:30341410 |
| 1188 | 1:30639309 | 1228 | 1:31833724 | 1232 | 1:32038632 | 1239 | 1:32383066 | 1245 | 1:32604779 |
| 1302 | 1:34248126 | 1303 | 1:34341221 | 1336 | 1:35240562 | 1357 | 1:35661327 | 1373 | 1:36152445 |
| 1491 | 1:39146665 | 1500 | 1:39465349 | 1501 | 1:39555945 | 1528 | 1:40302747 | 1529 | 1:40392357 |
| 1555 | 1:41086735 | 1563 | 1:41385809 | 1565 | 1:41492237 | 1626 | 1:42693702 | 1627 | 1:42705366 |
| 1628 | 1:42797258 | 1769 | 1:45607475 | 1771 | 1:45687245 | 1772 | 1:45787243 | 1818 | 1:47287965 |
| 1838 | 1:47969845 | 1845 | 1:48250419 | 1876 | 1:49256468 | 1899 | 1:50064432 | 1921 | 1:50739583 |
| 1956 | 1:51942050 | 1961 | 1:52098640 | 2014 | 1:53514840 | 2090 | 1:54855775 | 2101 | 1:55175239 |
| 2117 | 1:55765709 | 2124 | 1:55975682 | 2125 | 1:56071064 | 2227 | 1:58399681 | 2228 | 1:58497604 |
| 2264 | 1:59389492 | 2269 | 1:59650598 | 2357 | 1:61707467 | 2364 | 1:61908265 | 2533 | 1:65639354 |
| 2534 | 1:65691897 | 2542 | 1:65975858 | 2553 | 1:66261981 | 2554 | 1:66349787 | 2575 | 1:66970472 |
| 2601 | 1:67667419 | 2612 | 1:68106244 | 2693 | 1:69418925 | 2702 | 1:69664149 | 2804 | 1:71695300 |
| 2825 | 1:72263210 | 2865 | 1:73046338 | 2912 | 1:74307455 | 2913 | 1:74402077 | 2942 | 1:75146396 |
| 2992 | 1:76510120 | 3001 | 1:76727873 | 3014 | 1:77141699 | 3091 | 1:78826134 | 3171 | 1:80384212 |
| 3239 | 1:81998130 | 3282 | 1:82911267 | 3295 | 1:83439908 | 3307 | 1:83899382 | 3321 | 1:84316332 |
| 3356 | 1:85642906 | 3372 | 1:86015500 | 3389 | 1:86437496 | 3390 | 1:86526242 | 3403 | 1:86930423 |
| 3415 | 1:87291852 | 3447 | 1:88278205 | 3448 | 1:88303482 | 3449 | 1:88477613 | 3450 | 1:88510142 |
| 3451 | 1:88605108 | 3480 | 1:89106677 | 3481 | 1:89184242 | 3605 | 1:92127457 | 3615 | 1:92303587 |
| 3627 | 1:92748568 | 3631 | 1:92911886 | 3662 | 1:93749721 | 3788 | 1:96724055 | 3898 | 1:99648631 |
| 3899 | 1:99745058 | 3907 | 1:99964213 | 3912 | 1:100151147 | 3951 | 1:101402516 | 3959 | 1:101604960 |
| 3975 | 1:102178820 | 3985 | 1:102499753 | 4011 | 1:103210827 | 4043 | 1:104445688 | 4047 | 1:104601851 |
| 4128 | 1:106679632 | 4215 | 1:108995880 | 4218 | 1:109138566 | 4223 | 1:109344061 | 4244 | 1:109812035 |
| 4303 | 1:110891497 | 4316 | 1:111431902 | 4391 | 1:113109748 | 4461 | 1:114671853 | 4507 | 1:116130150 |
| 4509 | 1:116269280 | 4510 | 1:116368715 | 4688 | 1:119719715 | 4739 | 1:120951015 | 4741 | 1:121018439 |
| 4742 | 1:121074170 | 4743 | 1:121144456 | 4745 | 1:121224156 | 4749 | 1:121384190 | 4759 | 1:121640449 |
| 4764 | 1:121788170 | 4774 | 1:122051101 | 4832 | 1:123574290 | 4833 | 1:123657947 | 4976 | 1:126688131 |
| 4977 | 1:126726689 | 4978 | 1:126823765 | 5040 | 1:128486623 | 5043 | 1:128606933 | 5093 | 1:129682985 |
| 5098 | 1:129814612 | 5099 | 1:129913741 | 5241 | 1:132429392 | 5247 | 1:132635025 | 5251 | 1:132828896 |
| 5260 | 1:133234769 | 5284 | 1:134043255 | 5414 | 1:136299174 | 5432 | 1:136970680 | 5444 | 1:137364879 |
| 5459 | 1:138082957 | 5460 | 1:138180043 | 5462 | 1:138259482 | 5464 | 1:138404453 | 5500 | 1:139245663 |
| 5502 | 1:139304863 | 5503 | 1:139403568 | 5511 | 1:139653452 | 5578 | 1:141255563 | 5701 | 1:143320922 |
| 5754 | 1:144299047 | 5884 | 1:146674083 | 5885 | 1:146773640 | 6509 | 1:157687863 | 6510 | 1:157775988 |
| 6512 | 1:157881491 | 6523 | 1:158316722 | 6524 | 1:158361834 | 6525 | 1:158460274 | 6527 | 2:97987 |
| 6528 | 2:156099 | 6529 | 2:251841 | 6609 | 2:1676588 | 6610 | 2:1762272 | 6627 | 2:2208427 |
| 6711 | 2:4459297 | 6819 | 2:6376890 | 6897 | 2:7947030 | 6898 | 2:7984204 | 6899 | 2:8067862 |
| 6921 | 2:8713754 | 6943 | 2:9417024 | 6945 | 2:9540631 | 6961 | 2:9879504 | 6962 | 2:9977673 |
| 6990 | 2:10739801 | 6994 | 2:11002563 | 7030 | 2:12006500 | 7035 | 2:12186267 | 7046 | 2:12679896 |
| 7117 | 2:14600625 | 7167 | 2:15807478 | 7249 | 2:17949516 | 7391 | 2:20586534 | 7405 | 2:20982246 |
| 7482 | 2:22953223 | 7490 | 2:23131851 | 7539 | 2:24040103 | 7590 | 2:25076321 | 7805 | 2:29677272 |
| 7836 | 2:30463341 | 7892 | 2:31678013 | 7912 | 2:32367398 | 7928 | 2:32964948 | 8001 | 2:34879480 |
| 8002 | 2:34940227 | 8003 | 2:35037207 | 8015 | 2:35478468 | 8021 | 2:35685945 | 8062 | 2:36835120 |
| 8185 | 2:40374618 | 8195 | 2:40590316 | 8364 | 2:44741204 | 8381 | 2:45256936 | 8524 | 2:48700283 |
| 8548 | 2:49196955 | 8550 | 2:49308768 | 8552 | 2:49502134 | 8577 | 2:50260674 | 8621 | 2:51619705 |
| 8622 | 2:51714357 | 8648 | 2:52249299 | 8665 | 2:52682136 | 8707 | 2:53776420 | 8717 | 2:54059294 |
| 8723 | 2:54299723 | 8817 | 2:56671164 | 8818 | 2:56766656 | 8859 | 2:57934056 | 8886 | 2:58700978 |
| 8907 | 2:59199942 | 8956 | 2:60394889 | 8972 | 2:60962014 | 9010 | 2:61641610 | 9011 | 2:61738618 |
| 9018 | 2:62012037 | 9023 | 2:62250012 | 9101 | 2:64316198 | 9116 | 2:64570288 | 9246 | 2:68272329 |
| 9258 | 2:68690400 | 9261 | 2:68901070 | 9264 | 2:69034698 | 9269 | 2:69214365 | 9274 | 2:69379425 |
| 9323 | 2:70760400 | 9362 | 2:71684921 | 9363 | 2:71782525 | 9365 | 2:71848257 | 9367 | 2:71970045 |
| 9406 | 2:73054461 | 9417 | 2:73255223 | 9483 | 2:75058828 | 9582 | 2:77785420 | 9651 | 2:79212662 |
| 9652 | 2:79232935 | 9655 | 2:79435179 | 9688 | 2:80269896 | 9691 | 2:80350552 | 9692 | 2:80443906 |
| 9718 | 2:81298125 | 9721 | 2:81552356 | 9731 | 2:82160162 | 9738 | 2:82525466 | 9741 | 2:82703708 |
| 9757 | 2:83398924 | 9759 | 2:83488349 | 9790 | 2:84070506 | 9793 | 2:84186911 | 9820 | 2:84735988 |
| 9822 | 2:84748768 | 9823 | 2:84845633 | 9848 | 2:85539387 | 9873 | 2:86113417 | 9878 | 2:86175227 |
| 9879 | 2:86265979 | 9908 | 2:86950234 | 9953 | 2:88328898 | 9980 | 2:88998896 | 10067 | 2:90047120 |
| 10069 | 2:90182742 | 10070 | 2:90271127 | 10071 | 2:90346277 | 10107 | 2:91439705 | 10170 | 2:92960490 |
| 10178 | 2:93253701 | 10243 | 2:94504077 | 10356 | 2:96858239 | 10362 | 2:97119985 | 10364 | 2:97284470 |
| 10374 | 2:97660903 | 10388 | 2:97949623 | 10453 | 2:99661757 | 10479 | 2:100148811 | 10510 | 2:101083832 |
| 10528 | 2:101669777 | 10534 | 2:101822460 | 10562 | 2:102640055 | 10642 | 2:104771582 | 10671 | 2:105313365 |
| 10916 | 2:110949976 | 10930 | 2:111452102 | 11063 | 2:114575713 | 11083 | 2:115163293 | 11148 | 2:116629100 |
| 11191 | 2:117806168 | 11194 | 2:117939332 | 11231 | 2:118820218 | 11232 | 2:118916665 | 11274 | 2:119675734 |
| 11302 | 2:120419474 | 11330 | 2:121151600 | 11342 | 2:121489660 | 11349 | 2:121623817 | 11350 | 2:121707578 |
| 11363 | 2:122105560 | 11448 | 2:124381727 | 11449 | 2:124471534 | 11500 | 2:125909343 | 11658 | 2:128226965 |
| 11765 | 2:130549076 | 11809 | 2:131471648 | 12088 | 2:135958653 | 12089 | 2:136042952 | 12096 | 2:136122518 |
| 12097 | 2:136218516 | 12103 | 3:192288 | 12172 | 3:1379812 | 12218 | 3:2245775 | 12219 | 3:2335779 |
| 12428 | 3:6422650 | 12571 | 3:8746180 | 12573 | 3:8864644 | 12579 | 3:9185006 | 12699 | 3:10683176 |
| 12753 | 3:11779967 | 12757 | 3:11849178 | 12758 | 3:11934240 | 12759 | 3:11998748 | 12811 | 3:13516552 |
| 12962 | 3:16327244 | 13006 | 3:17313416 | 13007 | 3:17408532 | 13036 | 3:18384479 | 13087 | 3:19384406 |
| 13088 | 3:19480721 | 13103 | 3:19938418 | 13104 | 3:19975811 | 13105 | 3:20062027 | 13114 | 3:20219541 |
| 13115 | 3:20275444 | 13134 | 3:20949426 | 13135 | 3:21041525 | 13139 | 3:21260248 | 13153 | 3:21701072 |
| 13164 | 3:21935930 | 13203 | 3:22637339 | 13253 | 3:23859633 | 13388 | 3:26747082 | 13484 | 3:28324475 |
| 13485 | 3:28415348 | 13486 | 3:28426219 | 13487 | 3:28519784 | 13535 | 3:29477079 | 14013 | 3:38769939 |
| 14015 | 3:38908661 | 14018 | 3:39010474 | 14019 | 3:39102130 | 14042 | 3:39825859 | 14046 | 3:39962964 |
| 14061 | 3:40312602 | 14077 | 3:40804040 | 14079 | 3:40921947 | 14112 | 3:41900576 | 14171 | 3:43138350 |
| 14186 | 3:43521434 | 14223 | 3:44490132 | 14224 | 3:44561235 | 14250 | 3:45348826 | 14257 | 3:45588541 |
| 14268 | 3:45973111 | 14270 | 3:46106986 | 14307 | 3:47132786 | 14310 | 3:47350830 | 14401 | 3:49233756 |
| 14490 | 3:50263076 | 14511 | 3:50885592 | 14519 | 3:51298193 | 14525 | 3:51591043 | 14548 | 3:52087305 |
| 14576 | 3:52675804 | 14622 | 3:53900489 | 14624 | 3:54093636 | 14635 | 3:54224439 | 14636 | 3:54256413 |
| 14637 | 3:54344299 | 14639 | 3:54400470 | 14640 | 3:54475982 | 14641 | 3:54564882 | 14642 | 3:54618300 |
| 14643 | 3:54697466 | 14644 | 3:54793255 | 14645 | 3:54888122 | 14679 | 3:55617345 | 14680 | 3:55712074 |
| 14682 | 3:55757845 | 14683 | 3:55857361 | 14699 | 3:56411615 | 14703 | 3:56614407 | 14724 | 3:57120778 |
| 14725 | 3:57210410 | 14730 | 3:57316623 | 14763 | 3:58252296 | 14807 | 3:59522050 | 14820 | 3:59954303 |
| 14878 | 3:61139535 | 14937 | 3:62995597 | 14986 | 3:64406567 | 15080 | 3:66682938 | 15081 | 3:66782644 |
| 15094 | 3:67125573 | 15179 | 3:69231454 | 15232 | 3:70770801 | 15263 | 3:72024952 | 15274 | 3:72399723 |
| 15277 | 3:72561296 | 15278 | 3:72658767 | 15282 | 3:72860663 | 15323 | 3:74163128 | 15334 | 3:74460656 |
| 15335 | 3:74560466 | 15373 | 3:75613962 | 15388 | 3:75959257 | 15396 | 3:76253283 | 15407 | 3:76546947 |
| 15518 | 3:79853923 | 15527 | 3:80073268 | 15551 | 3:80845482 | 15603 | 3:82127843 | 15647 | 3:83276598 |
| 15687 | 3:84374682 | 15749 | 3:85174617 | 15864 | 3:87868064 | 16131 | 3:93389797 | 16146 | 3:93816462 |
| 16173 | 3:94470905 | 16174 | 3:94570511 | 16205 | 3:95542077 | 16234 | 3:96375723 | 16236 | 3:96516771 |
| 16319 | 3:98506422 | 16487 | 3:101953130 | 16551 | 3:103287865 | 17051 | 3:112888751 | 17107 | 3:113766186 |
| 17462 | 3:119142510 | 17463 | 3:119241254 | 17465 | 3:119312183 | 17466 | 3:119337850 | 17471 | 3:119559889 |
| 17490 | 3:120011571 | 17505 | 3:120402369 | 17508 | 3:120529061 | 17527 | 4:196536 | 17528 | 4:280153 |
| 17533 | 4:535625 | 17539 | 4:791948 | 17549 | 4:1260383 | 17552 | 4:1417558 | 17600 | 4:2614737 |
| 17745 | 4:5912982 | 17995 | 4:11174189 | 18042 | 4:12473770 | 18087 | 4:13462303 | 18127 | 4:14704975 |
| 18131 | 4:14787436 | 18168 | 4:15690889 | 18178 | 4:16124536 | 18250 | 4:17859359 | 18322 | 4:19353388 |
| 18383 | 4:20445754 | 18433 | 4:21454127 | 18434 | 4:21541657 | 18445 | 4:21872378 | 18462 | 4:22281058 |
| 18501 | 4:23239526 | 18518 | 4:23775334 | 18529 | 4:24057792 | 18561 | 4:25063606 | 18581 | 4:25549415 |
| 18709 | 4:28892826 | 18713 | 4:29073099 | 18729 | 4:29642872 | 18740 | 4:30014475 | 18741 | 4:30101996 |
| 18846 | 4:32916549 | 18904 | 4:34496286 | 18926 | 4:35328524 | 19007 | 4:37643101 | 19054 | 4:38955171 |
| 19106 | 4:40318271 | 19156 | 4:41249802 | 19160 | 4:41529195 | 19161 | 4:41619769 | 19165 | 4:41769265 |
| 19169 | 4:41869434 | 19170 | 4:41950512 | 19184 | 4:42417863 | 19237 | 4:43892488 | 19281 | 4:45005168 |
| 19322 | 4:46242543 | 19325 | 4:46409911 | 19326 | 4:46490707 | 19407 | 4:48377180 | 19408 | 4:48467232 |
| 19487 | 4:50087185 | 19488 | 4:50186156 | 19499 | 4:50633232 | 19510 | 4:50996653 | 19537 | 4:51864991 |
| 19612 | 4:53951273 | 19613 | 4:54035561 | 19616 | 4:54191462 | 19618 | 4:54332820 | 19619 | 4:54391051 |
| 19629 | 4:54743084 | 19640 | 4:55160033 | 19642 | 4:55344926 | 19687 | 4:56506421 | 19766 | 4:59069599 |
| 19793 | 4:59746110 | 19805 | 4:60065778 | 19828 | 4:60746730 | 19861 | 4:61706369 | 19892 | 4:62405503 |
| 19897 | 4:62630427 | 19898 | 4:62713783 | 19987 | 4:65201660 | 19989 | 4:65245742 | 19990 | 4:65343359 |
| 19991 | 4:65436101 | 19993 | 4:65541264 | 20071 | 4:67301224 | 20088 | 4:67785324 | 20101 | 4:68075914 |
| 20135 | 4:69146493 | 20160 | 4:69711048 | 20161 | 4:69810925 | 20204 | 4:70819803 | 20231 | 4:71683652 |
| 20252 | 4:72269219 | 20253 | 4:72368269 | 20301 | 4:73885846 | 20309 | 4:74101412 | 20450 | 4:77371853 |
| 20475 | 4:77790836 | 20529 | 4:79467619 | 20647 | 4:82182401 | 20730 | 4:84549829 | 20733 | 4:84649340 |
| 20835 | 4:87133726 | 20859 | 4:87827889 | 20910 | 4:89303585 | 20944 | 4:90250683 | 20961 | 4:90701139 |
| 20978 | 4:91069757 | 21084 | 4:93203600 | 21136 | 4:94310305 | 21160 | 4:95074007 | 21254 | 4:96620212 |
| 21255 | 4:96718455 | 21485 | 4:99029738 | 21489 | 4:99310528 | 21491 | 4:99423168 | 21610 | 4:101894551 |
| 21616 | 4:102178027 | 21696 | 4:104152919 | 21762 | 4:105592184 | 21763 | 4:105606470 | 21764 | 4:105706248 |
| 21771 | 4:105878830 | 21772 | 4:105960151 | 21945 | 4:109512073 | 22167 | 4:113058361 | 22168 | 4:113156968 |
| 22546 | 4:119885612 | 22565 | 5:195803 | 22566 | 5:239346 | 22567 | 5:334945 | 22569 | 5:484209 |
| 22570 | 5:577221 | 22620 | 5:1547817 | 22706 | 5:3643809 | 22817 | 5:6128913 | 22818 | 5:6209013 |
| 23050 | 5:11616643 | 23133 | 5:13446994 | 23146 | 5:13786322 | 23170 | 5:14539492 | 23173 | 5:14638005 |
| 23183 | 5:14893917 | 23248 | 5:16007217 | 23258 | 5:16431067 | 23300 | 5:17571373 | 23303 | 5:17775869 |
| 23312 | 5:18094567 | 23332 | 5:18790466 | 23643 | 5:26034515 | 23644 | 5:26061374 | 23645 | 5:26151322 |
| 23657 | 5:26429773 | 23772 | 5:28790811 | 23781 | 5:29074134 | 23866 | 5:30986242 | 23867 | 5:31079915 |
| 23893 | 5:31837129 | 23983 | 5:34087072 | 23986 | 5:34286241 | 23987 | 5:34305687 | 23988 | 5:34375513 |
| 23989 | 5:34474025 | 23993 | 5:34687154 | 24020 | 5:35185968 | 24025 | 5:35351867 | 24034 | 5:35659101 |
| 24055 | 5:36320021 | 24070 | 5:36698940 | 24073 | 5:36843791 | 24100 | 5:37690831 | 24156 | 5:38824022 |
| 24222 | 5:40535193 | 24224 | 5:40667896 | 24269 | 5:42106898 | 24276 | 5:42475432 | 24298 | 5:42980132 |
| 24308 | 5:43193547 | 24330 | 5:43788410 | 24355 | 5:44185303 | 24356 | 5:44283317 | 24411 | 5:45870645 |
| 24496 | 5:47715438 | 24508 | 5:48068765 | 24588 | 5:50328496 | 24634 | 5:51301107 | 24681 | 5:52618260 |
| 24684 | 5:52742025 | 24687 | 5:52972061 | 24709 | 5:53722470 | 24719 | 5:53987712 | 24800 | 5:55749160 |
| 24856 | 5:57057069 | 24875 | 5:57516539 | 24885 | 5:57746077 | 24886 | 5:57832714 | 24887 | 5:57930196 |
| 24906 | 5:58383434 | 24909 | 5:58493473 | 24913 | 5:58628294 | 24914 | 5:58693980 | 24917 | 5:58889899 |
| 24918 | 5:58986587 | 24932 | 5:59477581 | 24935 | 5:59606022 | 24937 | 5:59692009 | 24938 | 5:59786270 |
| 24966 | 5:60382341 | 24967 | 5:60450259 | 25017 | 5:61851862 | 25115 | 5:64573229 | 25212 | 5:67004367 |
| 25214 | 5:67099597 | 25215 | 5:67183935 | 25245 | 5:67610309 | 25287 | 5:68696282 | 25352 | 5:70049824 |
| 25368 | 5:70612128 | 25480 | 5:72974385 | 25499 | 5:73530687 | 25534 | 5:74251346 | 25535 | 5:74343381 |
| 25593 | 5:75456468 | 25594 | 5:75473431 | 25595 | 5:75561363 | 25668 | 5:77473876 | 25669 | 5:77528763 |
| 25676 | 5:77903820 | 25690 | 5:78146870 | 25691 | 5:78242136 | 25730 | 5:79116954 | 25737 | 5:79370071 |
| 25738 | 5:79464943 | 25739 | 5:79516884 | 25762 | 5:80354597 | 25763 | 5:80452900 | 25786 | 5:81078405 |
| 25790 | 5:81259940 | 25866 | 5:82675565 | 25872 | 5:82889086 | 26084 | 5:84839297 | 26119 | 5:85681634 |
| 26136 | 5:86133720 | 26140 | 5:86239067 | 26194 | 5:87412820 | 26322 | 5:89485893 | 26392 | 5:91260025 |
| 26475 | 5:92686818 | 26708 | 5:97550215 | 26787 | 5:99116271 | 26788 | 5:99137116 | 26789 | 5:99143867 |
| 26790 | 5:99243441 | 26791 | 5:99314005 | 26931 | 5:102029541 | 26933 | 5:102150740 | 26938 | 5:102366923 |
| 26949 | 5:102638331 | 26950 | 5:102641378 | 26951 | 5:102662950 | 26952 | 5:102812214 | 26953 | 5:102878588 |
| 26954 | 5:102970619 | 26955 | 5:103010571 | 27120 | 5:106009983 | 27279 | 5:108384166 | 27298 | 5:108674540 |
| 27332 | 5:109217271 | 27333 | 5:109314750 | 27544 | 5:113514180 | 27545 | 5:113554598 | 27575 | 5:114440055 |
| 27576 | 5:114536143 | 27578 | 5:114640058 | 27595 | 5:115151924 | 27738 | 5:116870418 | 27938 | 6:97487 |
| 27946 | 6:394469 | 27947 | 6:492748 | 27979 | 6:1193084 | 28037 | 6:2239422 | 28060 | 6:2768563 |
| 28130 | 6:3837662 | 28184 | 6:5212458 | 28195 | 6:5517626 | 28196 | 6:5615037 | 28314 | 6:8143447 |
| 28328 | 6:8615351 | 28329 | 6:8684262 | 28437 | 6:11262662 | 28551 | 6:13520510 | 28553 | 6:13697509 |
| 28571 | 6:14245516 | 28744 | 6:17861677 | 28918 | 6:21562233 | 29086 | 6:25221507 | 29099 | 6:25661883 |
| 29133 | 6:26462640 | 29181 | 6:27717652 | 29384 | 6:31231051 | 29419 | 6:32134214 | 29424 | 6:32438836 |
| 29434 | 6:32698791 | 29437 | 6:32783759 | 29438 | 6:32866982 | 32354 | 6:44532925 | 32523 | 6:45804379 |
| 32559 | 6:46543851 | 32571 | 6:46849949 | 32625 | 6:48139435 | 32639 | 6:48619247 | 32722 | 6:50593610 |
| 32763 | 6:51940574 | 32772 | 6:52186933 | 32883 | 6:54324568 | 32961 | 6:55908779 | 32979 | 6:56522044 |
| 33028 | 6:57631443 | 33029 | 6:57728837 | 33054 | 6:58492711 | 33067 | 6:58822636 | 33096 | 6:59668604 |
| 33161 | 6:61063375 | 33203 | 6:62168026 | 33214 | 6:62509742 | 33219 | 6:62716293 | 33266 | 6:64072884 |
| 33270 | 6:64250794 | 33339 | 6:66605824 | 33359 | 6:67153345 | 33441 | 6:69518831 | 33443 | 6:69655680 |
| 33453 | 6:69965893 | 33454 | 6:70058818 | 33456 | 6:70166659 | 33473 | 6:70643646 | 33511 | 6:71802143 |
| 33591 | 6:74410889 | 33596 | 6:74629845 | 33599 | 6:74741424 | 33617 | 6:75350029 | 33619 | 6:75446552 |
| 33621 | 6:75621675 | 33636 | 6:75990894 | 33639 | 6:76209600 | 33640 | 6:76275919 | 33643 | 6:76450277 |
| 33706 | 6:78238972 | 33708 | 6:78363832 | 33713 | 6:78527838 | 33724 | 6:79038243 | 33745 | 6:79831508 |
| 33795 | 6:81332996 | 33807 | 6:81870711 | 33808 | 6:81964290 | 33862 | 6:83422593 | 33883 | 6:83749295 |
| 33906 | 6:84274386 | 33923 | 6:84775788 | 33989 | 6:86113898 | 34365 | 6:93730394 | 34406 | 6:94508641 |
| 34407 | 6:94604426 | 34408 | 6:94671907 | 34418 | 6:95161943 | 34422 | 6:95280868 | 34621 | 6:99278886 |
| 34645 | 6:100084140 | 34895 | 6:104783662 | 34926 | 6:105376492 | 35015 | 6:107674685 | 35016 | 6:107769675 |
| 35048 | 6:108791274 | 35098 | 6:109783321 | 35256 | 6:113049419 | 35257 | 6:113144284 | 35385 | 6:115461946 |
| 35450 | 6:116698963 | 35451 | 6:116752334 | 35452 | 6:116848436 | 35454 | 6:117023687 | 35455 | 6:117121869 |
| 35460 | 6:117289738 | 35461 | 6:117373173 | 35468 | 6:117512770 | 35471 | 6:117661215 | 35472 | 6:117698401 |
| 35473 | 6:117792640 | 35474 | 7:80741 | 35475 | 7:164546 | 35720 | 7:4691508 | 35922 | 7:7350133 |
| 35945 | 7:8005827 | 35954 | 7:8250300 | 35957 | 7:8423976 | 35962 | 7:8644832 | 35963 | 7:8715092 |
| 35964 | 7:8798532 | 35965 | 7:8829774 | 35967 | 7:9006597 | 35968 | 7:9034045 | 35969 | 7:9111685 |
| 35970 | 7:9205519 | 35971 | 7:9294350 | 35972 | 7:9323285 | 35973 | 7:9420386 | 35974 | 7:9427375 |
| 35975 | 7:9522686 | 35976 | 7:9608447 | 35980 | 7:9782711 | 35981 | 7:9853483 | 35982 | 7:9893322 |
| 35983 | 7:9935560 | 35987 | 7:10196676 | 35988 | 7:10292183 | 35993 | 7:10432630 | 35994 | 7:10494698 |
| 35995 | 7:10588854 | 36000 | 7:10694692 | 36001 | 7:10709248 | 36002 | 7:10799397 | 36003 | 7:10898948 |
| 36004 | 7:10972220 | 36034 | 7:11621411 | 36038 | 7:11733951 | 36290 | 7:15957756 | 36351 | 7:17374656 |
| 36373 | 7:17800196 | 36382 | 7:18107639 | 36530 | 7:20567856 | 36589 | 7:21886187 | 36612 | 7:22364050 |
| 36643 | 7:23141409 | 36693 | 7:24266298 | 36997 | 7:31102265 | 37168 | 7:34931021 | 37197 | 7:35685794 |
| 37213 | 7:36102653 | 37218 | 7:36242365 | 37331 | 7:38727405 | 37345 | 7:39057017 | 37432 | 7:40812159 |
| 37449 | 7:41102749 | 37450 | 7:41174068 | 37456 | 7:41391176 | 37460 | 7:41530820 | 37465 | 7:41621787 |
| 37485 | 7:42209152 | 37489 | 7:42394156 | 37545 | 7:43685836 | 37553 | 7:43941722 | 37559 | 7:44261005 |
| 37564 | 7:44505025 | 37616 | 7:45789210 | 37618 | 7:45899026 | 37622 | 7:46066923 | 37702 | 7:47884765 |
| 37704 | 7:47992422 | 37706 | 7:48152895 | 37757 | 7:49264671 | 37763 | 7:49508927 | 37764 | 7:49551981 |
| 37765 | 7:49632647 | 37766 | 7:49732360 | 37767 | 7:49825415 | 37772 | 7:50083648 | 37773 | 7:50141438 |
| 37774 | 7:50238296 | 37775 | 7:50295962 | 37776 | 7:50392227 | 37778 | 7:50523822 | 37782 | 7:50734140 |
| 37785 | 7:50857210 | 37786 | 7:50956025 | 37795 | 7:51298758 | 37805 | 7:51627529 | 37808 | 7:51770556 |
| 37810 | 7:51904626 | 37877 | 7:52516644 | 37913 | 7:53384544 | 37926 | 7:53682528 | 37936 | 7:54073527 |
| 38031 | 7:56472724 | 38061 | 7:57477755 | 38086 | 7:58105186 | 38087 | 7:58204392 | 38167 | 7:59670910 |
| 38195 | 7:60290921 | 38291 | 7:62241248 | 38363 | 7:63608853 | 38381 | 7:64051258 | 38502 | 7:66283774 |
| 38508 | 7:66535109 | 38570 | 7:68229559 | 38620 | 7:69315564 | 38662 | 7:70240253 | 38678 | 7:70856473 |
| 38773 | 7:73340932 | 38774 | 7:73425725 | 38792 | 7:73921199 | 38812 | 7:74461479 | 39002 | 7:78855864 |
| 39182 | 7:82082096 | 39313 | 7:85461499 | 39332 | 7:86158728 | 39339 | 7:86335874 | 39344 | 7:86445479 |
| 39366 | 7:86982142 | 39476 | 7:89945029 | 39477 | 7:90039805 | 39669 | 7:93367867 | 39716 | 7:94784594 |
| 40029 | 7:98642157 | 40031 | 7:98712459 | 40032 | 7:98793113 | 40136 | 7:101498466 | 40233 | 7:103750242 |
| 40234 | 7:103835986 | 40277 | 7:105099277 | 40483 | 7:110118836 | 40484 | 7:110198554 | 40485 | 7:110298545 |
| 40486 | 7:110364995 | 40487 | 7:110462011 | 40488 | 7:110524775 | 40489 | 7:110616075 | 40491 | 8:119077 |
| 40492 | 8:204055 | 40502 | 8:510254 | 40549 | 8:1447144 | 40550 | 8:1539600 | 40593 | 8:2298980 |
| 40618 | 8:3109616 | 40678 | 8:4419528 | 40679 | 8:4488605 | 40713 | 8:5356367 | 40792 | 8:7353398 |
| 40816 | 8:7715961 | 40887 | 8:9573171 | 40913 | 8:10172186 | 41018 | 8:12543611 | 41019 | 8:12642666 |
| 41081 | 8:14746899 | 41091 | 8:15001201 | 41101 | 8:15260009 | 41198 | 8:17406666 | 41267 | 8:19472727 |
| 41298 | 8:20253347 | 41380 | 8:22549276 | 41383 | 8:22747845 | 41388 | 8:22913643 | 41395 | 8:23313119 |
| 41446 | 8:24571671 | 41627 | 8:27179633 | 41661 | 8:28342382 | 41677 | 8:28819278 | 41709 | 8:29645861 |
| 41763 | 8:30913973 | 41814 | 8:32226362 | 41825 | 8:32643213 | 41830 | 8:32879092 | 41831 | 8:32974702 |
| 41833 | 8:33127475 | 41844 | 8:33407993 | 41845 | 8:33470771 | 41900 | 8:34950717 | 41905 | 8:35118252 |
| 41930 | 8:35658609 | 42020 | 8:37747312 | 42042 | 8:38560565 | 42053 | 8:38837520 | 42054 | 8:38936996 |
| 42058 | 8:39131324 | 42203 | 8:42579916 | 42214 | 8:42818923 | 42264 | 8:44335672 | 42287 | 8:45011011 |
| 42294 | 8:45233429 | 42414 | 8:47856381 | 42435 | 8:48611381 | 42541 | 8:51528439 | 42598 | 8:52866048 |
| 42705 | 8:55793930 | 42720 | 8:56175135 | 42793 | 8:57947632 | 42804 | 8:58378680 | 42806 | 8:58511840 |
| 42900 | 8:60710884 | 42937 | 8:61860162 | 42990 | 8:63095889 | 42996 | 8:63293941 | 42997 | 8:63391647 |
| 43046 | 8:64688332 | 43070 | 8:65351408 | 43083 | 8:65593070 | 43086 | 8:65766665 | 43108 | 8:66276105 |
| 43137 | 8:66960690 | 43199 | 8:68392696 | 43246 | 8:69535824 | 43279 | 8:70389972 | 43369 | 8:72702268 |
| 43466 | 8:75072947 | 43494 | 8:76094638 | 43555 | 8:77086614 | 43558 | 8:77243628 | 43569 | 8:77734663 |
| 43571 | 8:77844702 | 43601 | 8:78788179 | 43610 | 8:79140878 | 43615 | 8:79417890 | 43732 | 8:82254632 |
| 43780 | 8:83455155 | 43784 | 8:83630961 | 43864 | 8:85507118 | 43904 | 8:86456661 | 43907 | 8:86622904 |
| 43962 | 8:87959678 | 43976 | 8:88341264 | 44029 | 8:89430089 | 44104 | 8:91188437 | 44105 | 8:91284358 |
| 44119 | 8:91608918 | 44148 | 8:92091641 | 44201 | 8:93754674 | 44213 | 8:94306363 | 44214 | 8:94402192 |
| 44215 | 8:94469316 | 44280 | 8:96346983 | 44433 | 8:99864196 | 44481 | 8:101198784 | 44501 | 8:101698974 |
| 44683 | 8:104921232 | 44740 | 8:106178029 | 44742 | 8:106321176 | 44817 | 8:108369912 | 44818 | 8:108466305 |
| 44826 | 8:108830921 | 44827 | 8:108930028 | 44840 | 8:109399618 | 44844 | 8:109663372 | 44876 | 8:110607864 |
| 44902 | 8:111508670 | 44903 | 8:111524854 | 44904 | 8:111672008 | 44905 | 8:111767142 | 44906 | 8:111818202 |
| 44907 | 8:111891683 | 44908 | 8:111919894 | 44909 | 8:112058818 | 44910 | 8:112150362 | 44911 | 8:112232760 |
| 44912 | 8:112294473 | 44913 | 8:112461632 | 44914 | 8:112537285 | 44915 | 8:112604404 | 44916 | 8:112658175 |
| 44917 | 8:112746559 | 44918 | 8:112818233 | 44919 | 8:112865068 | 44920 | 8:112932415 | 44921 | 8:112969679 |
| 44922 | 8:112981176 | 44923 | 8:113097953 | 44924 | 8:113163827 | 44925 | 8:113180320 | 44926 | 8:113252524 |
| 44930 | 9:190038 | 44931 | 9:289864 | 45031 | 9:2539725 | 45074 | 9:3743248 | 45089 | 9:4213848 |
| 45112 | 9:4988015 | 45135 | 9:5683174 | 45179 | 9:7188401 | 45252 | 9:8906729 | 45274 | 9:9555271 |
| 45378 | 9:10911653 | 45502 | 9:13481091 | 45518 | 9:13965555 | 45582 | 9:15291327 | 45748 | 9:19358233 |
| 45798 | 9:20933619 | 45807 | 9:21235916 | 45808 | 9:21329366 | 46023 | 9:26340073 | 46029 | 9:26560747 |
| 46194 | 9:29938464 | 46280 | 9:31821673 | 46371 | 9:34153811 | 46384 | 9:34492853 | 46503 | 9:37375637 |
| 46515 | 9:37681606 | 46604 | 9:39416078 | 46605 | 9:39496518 | 46691 | 9:41269453 | 46692 | 9:41353434 |
| 46707 | 9:41741270 | 46708 | 9:41834289 | 46710 | 9:41949048 | 46773 | 9:43557020 | 46780 | 9:43780420 |
| 46834 | 9:45079509 | 46835 | 9:45168173 | 46839 | 9:45353183 | 46856 | 9:45976363 | 46867 | 9:46356308 |
| 46879 | 9:46772812 | 46892 | 9:47186026 | 46935 | 9:48538260 | 47033 | 9:51244082 | 47100 | 9:53108085 |
| 47125 | 9:53577095 | 47177 | 9:55074875 | 47178 | 9:55120285 | 47179 | 9:55219943 | 47205 | 9:56052958 |
| 47226 | 9:56606356 | 47254 | 9:57407794 | 47256 | 9:57496462 | 47281 | 9:58031623 | 47311 | 9:59018652 |
| 47408 | 9:61120108 | 47411 | 9:61356232 | 47442 | 9:62281541 | 47443 | 9:62376661 | 47493 | 9:63781683 |
| 47496 | 9:63979099 | 47499 | 9:64121913 | 47535 | 9:64946776 | 47643 | 9:66726405 | 47644 | 9:66806876 |
| 47646 | 9:66932759 | 47655 | 9:67186941 | 47715 | 9:68688996 | 47850 | 9:71852129 | 47857 | 9:72065985 |
| 47858 | 9:72161733 | 47894 | 9:73058318 | 47968 | 9:74896771 | 48054 | 9:77165874 | 48179 | 9:79135168 |
| 48188 | 9:79446217 | 48426 | 9:84561403 | 48431 | 9:84647624 | 48432 | 9:84745238 | 48521 | 9:86545282 |
| 48523 | 9:86670733 | 48544 | 9:87047352 | 48545 | 9:87102465 | 48546 | 9:87196437 | 48547 | 9:87295910 |
| 48615 | 9:88318893 | 48799 | 9:91914972 | 48879 | 9:93747256 | 49154 | 9:98379141 | 49230 | 9:99867971 |
| 49483 | 9:104102942 | 49484 | 9:104107451 | 49486 | 9:104142058 | 49488 | 9:104277105 | 49495 | 9:104453514 |
| 49497 | 9:104550915 | 49498 | 9:104641226 | 49499 | 9:104641308 | 49500 | 9:104641495 | 49502 | 10:184460 |
| 49503 | 10:213217 | 49504 | 10:295234 | 49506 | 10:344867 | 49507 | 10:435671 | 49566 | 10:1764102 |
| 49836 | 10:6862089 | 49850 | 10:7338516 | 49919 | 10:8633030 | 49991 | 10:10372137 | 50045 | 10:11267448 |
| 50115 | 10:12952255 | 50434 | 10:18833922 | 50435 | 10:18841050 | 50436 | 10:18845495 | 50437 | 10:18848616 |
| 50438 | 10:18854031 | 50469 | 10:19429779 | 50470 | 10:19516186 | 50638 | 10:22676353 | 50639 | 10:22775635 |
| 50642 | 10:22836164 | 50643 | 10:22909428 | 50646 | 10:23059842 | 50647 | 10:23066056 | 50648 | 10:23228975 |
| 50649 | 10:23268766 | 50650 | 10:23368614 | 50652 | 10:23464004 | 50653 | 10:23533419 | 50654 | 10:23627755 |
| 50655 | 10:23671760 | 50656 | 10:23769788 | 50657 | 10:23771464 | 50658 | 10:24790738 | 50659 | 10:24799735 |
| 50660 | 10:24818929 | 50661 | 10:24913820 | 50662 | 10:24923736 | 50663 | 10:25022602 | 50664 | 10:25057985 |
| 50665 | 10:25156372 | 50666 | 10:25164147 | 50667 | 10:25241806 | 50668 | 10:25341513 | 50680 | 10:25758684 |
| 50778 | 10:27556986 | 50900 | 10:29890120 | 50926 | 10:30807403 | 51080 | 10:34810626 | 51145 | 10:36132543 |
| 51146 | 10:36213908 | 51160 | 10:36596113 | 51163 | 10:36787409 | 51240 | 10:38618727 | 51259 | 10:39073733 |
| 51260 | 10:39169136 | 51284 | 10:39715696 | 51292 | 10:39993149 | 51302 | 10:40306626 | 51390 | 10:42652463 |
| 51403 | 10:43115839 | 51417 | 10:43643966 | 51433 | 10:44096569 | 51474 | 10:45124642 | 51490 | 10:45511746 |
| 51745 | 10:50796271 | 51861 | 10:53660012 | 51942 | 10:56043883 | 51992 | 10:57210937 | 52035 | 10:58319989 |
| 52040 | 10:58481438 | 52054 | 10:58874663 | 52071 | 10:59336872 | 52092 | 10:60028136 | 52123 | 10:61231374 |
| 52126 | 10:61443371 | 52158 | 10:62042620 | 52193 | 10:62708109 | 52228 | 10:63495064 | 52236 | 10:63804854 |
| 52247 | 10:64189747 | 52294 | 10:65251624 | 52312 | 10:65561116 | 52344 | 10:66259714 | 52392 | 10:67327408 |
| 52523 | 10:70522773 | 52524 | 10:70601203 | 52526 | 10:70690873 | 52527 | 10:70781098 | 52548 | 10:71377715 |
| 52590 | 10:72631813 | 52591 | 10:72727860 | 52596 | 10:72983865 | 52597 | 10:73076966 | 52643 | 10:74135935 |
| 52664 | 10:74941316 | 52700 | 10:75976569 | 52736 | 10:76707628 | 53015 | 10:82572013 | 53034 | 10:83144860 |
| 53169 | 10:86010466 | 53423 | 10:91110721 | 53454 | 10:92010714 | 53519 | 10:93279984 | 53520 | 10:93370297 |
| 53640 | 10:96340328 | 53705 | 10:97729030 | 53779 | 10:99321961 | 53802 | 10:99841614 | 53981 | 10:10327740 |
| 54006 | 11:514378 | 54266 | 11:4322857 | 54313 | 11:5395664 | 54364 | 11:6596735 | 54435 | 11:8259962 |
| 54438 | 11:8462700 | 54446 | 11:8761089 | 54705 | 11:14474472 | 54822 | 11:17458053 | 54838 | 11:17908654 |
| 54839 | 11:17983413 | 54843 | 11:18162535 | 54847 | 11:18333639 | 54889 | 11:19456930 | 54900 | 11:19732194 |
| 54920 | 11:20390071 | 54993 | 11:22091427 | 55041 | 11:23355951 | 55079 | 11:24188498 | 55126 | 11:25171864 |
| 55148 | 11:25792655 | 55180 | 11:26465214 | 55181 | 11:26466574 | 55182 | 11:26531940 | 55361 | 11:30575172 |
| 55422 | 11:32139468 | 55488 | 11:33907492 | 55506 | 11:34748337 | 55580 | 11:36801995 | 55638 | 11:37982330 |
| 55648 | 11:38441743 | 55686 | 11:39673530 | 55712 | 11:40436923 | 55715 | 11:40647883 | 55794 | 11:42338919 |
| 55853 | 11:43645607 | 55866 | 11:44133002 | 55918 | 11:45401293 | 55920 | 11:45518720 | 55922 | 11:45703344 |
| 55923 | 11:45779776 | 55956 | 11:46344663 | 56060 | 11:48391618 | 56182 | 11:50484454 | 56211 | 11:51437117 |
| 56233 | 11:52229998 | 56236 | 11:52492485 | 56254 | 11:53121751 | 56260 | 11:53366816 | 56270 | 11:53745957 |
| 56342 | 11:55279305 | 56373 | 11:55996334 | 56379 | 11:56241704 | 56381 | 11:56406566 | 56386 | 11:56550578 |
| 56393 | 11:56780643 | 56413 | 11:57282162 | 56423 | 11:57597675 | 56424 | 11:57696611 | 56449 | 11:58487002 |
| 56502 | 11:59891961 | 56507 | 11:60131924 | 56508 | 11:60226569 | 56583 | 11:61202324 | 56584 | 11:61289988 |
| 56586 | 11:61410046 | 56587 | 11:61495664 | 56592 | 11:61644339 | 56593 | 11:61726332 | 56717 | 11:64661124 |
| 56765 | 11:65925707 | 56800 | 11:66728012 | 56888 | 11:68240104 | 56900 | 11:68629264 | 56946 | 11:69472443 |
| 57044 | 11:70953054 | 57115 | 11:72333655 | 57123 | 11:72679475 | 57165 | 11:73412263 | 57169 | 11:73601533 |
| 57220 | 11:74558447 | 57221 | 11:74602747 | 57223 | 11:74706334 | 57268 | 11:75776010 | 57283 | 11:76197120 |
| 57467 | 11:79506772 | 57675 | 11:84372612 | 57684 | 11:84807135 | 57765 | 11:86512168 | 57810 | 11:87538574 |
| 57811 | 11:87624731 | 57982 | 11:90885707 | 58110 | 11:93370799 | 58146 | 11:94432248 | 58203 | 11:95957372 |
| 58367 | 11:99599606 | 58410 | 11:10044124 | 58478 | 11:10165527 | 58566 | 11:10321963 | 58606 | 11:10400943 |
| 58677 | 11:10536644 | 58680 | 11:10545702 | 58708 | 11:10585665 | 58709 | 11:10595532 | 58714 | 11:10618317 |
| 58716 | 11:10629012 | 58718 | 11:10643100 | 58721 | 11:10656647 | 58728 | 11:10671924 | 58730 | 11:10687276 |
| 58732 | 12:91462 | 58765 | 12:1113931 | 58768 | 12:1245380 | 58884 | 12:3705269 | 58963 | 12:5912555 |
| 58975 | 12:6345821 | 58982 | 12:6709902 | 58998 | 12:7126322 | 58999 | 12:7224038 | 59220 | 12:12834462 |
| 59327 | 12:15203798 | 59530 | 12:19527022 | 59607 | 12:21552423 | 59610 | 12:21691586 | 59611 | 12:21787162 |
| 59641 | 12:22535408 | 59688 | 12:23635155 | 59689 | 12:23728488 | 59791 | 12:25996134 | 59792 | 12:26092741 |
| 59794 | 12:26216578 | 59804 | 12:26581859 | 59875 | 12:28358077 | 59905 | 12:29299176 | 59926 | 12:29941712 |
| 59950 | 12:30491646 | 60005 | 12:31739889 | 60020 | 12:32049680 | 60093 | 12:33950251 | 60098 | 12:34160176 |
| 60110 | 12:34560071 | 60129 | 12:35057478 | 60167 | 12:35870799 | 60171 | 12:36164862 | 60174 | 12:36313336 |
| 60188 | 12:36646069 | 60194 | 12:36915655 | 60217 | 12:37496437 | 60218 | 12:37593149 | 60219 | 12:37662796 |
| 60221 | 12:37752580 | 60223 | 12:37929010 | 60224 | 12:37976297 | 60225 | 12:38053259 | 60230 | 12:38238813 |
| 60242 | 12:38895814 | 60247 | 12:39058185 | 60250 | 12:39172935 | 60271 | 12:39830361 | 60272 | 12:39927124 |
| 60297 | 12:40565431 | 60302 | 12:40827682 | 60314 | 12:41265419 | 60318 | 12:41435539 | 60322 | 12:41606679 |
| 60337 | 12:42104689 | 60343 | 12:42399827 | 60348 | 12:42626305 | 60349 | 12:42724793 | 60353 | 12:42919011 |
| 60357 | 12:43020517 | 60359 | 12:43190819 | 60369 | 12:43612489 | 60370 | 12:43698277 | 60374 | 12:43993681 |
| 60398 | 12:44816702 | 60406 | 12:45079833 | 60408 | 12:45170993 | 60432 | 12:45962853 | 60433 | 12:46045130 |
| 60515 | 12:48438301 | 60537 | 12:48906417 | 60622 | 12:50638774 | 60623 | 12:50712724 | 60795 | 12:54440430 |
| 60879 | 12:56915140 | 60896 | 12:57520936 | 60897 | 12:57618510 | 60917 | 12:58198920 | 60927 | 12:58558724 |
| 60980 | 12:60328402 | 60984 | 12:60431728 | 60991 | 12:60833351 | 60998 | 12:61166007 | 61017 | 12:61818463 |
| 61069 | 12:63224791 | 61091 | 12:63866391 | 61114 | 12:64636179 | 61130 | 12:65016774 | 61202 | 12:66945480 |
| 61203 | 12:67032666 | 61345 | 12:69830668 | 61346 | 12:70008764 | 61347 | 12:70092121 | 61349 | 12:70199557 |
| 61350 | 12:70296346 | 61351 | 12:70314066 | 61352 | 12:70373860 | 61354 | 12:70528567 | 61355 | 12:70625414 |
| 61356 | 12:70723529 | 61359 | 12:70754288 | 61360 | 12:70832160 | 61361 | 12:70921442 | 61362 | 12:71020218 |
| 61363 | 12:71027334 | 61364 | 12:71106543 | 61365 | 12:71199759 | 61375 | 12:71408795 | 61376 | 12:71457224 |
| 61377 | 12:71551210 | 61384 | 12:71729441 | 61392 | 12:71965489 | 61393 | 12:72058978 | 61394 | 12:72147420 |
| 61395 | 12:72230980 | 61396 | 12:72265152 | 61397 | 12:72337871 | 61398 | 12:72374872 | 61399 | 12:72490055 |
| 61400 | 12:72498314 | 61401 | 12:72637442 | 61402 | 12:72712776 | 61403 | 12:72812633 | 61902 | 12:83578230 |
| 62097 | 12:87141742 | 62098 | 13:299895 | 62105 | 13:682778 | 62106 | 13:745802 | 62125 | 13:1154063 |
| 62224 | 13:2881031 | 62225 | 13:2975142 | 62280 | 13:4395240 | 62283 | 13:4531236 | 62431 | 13:8202540 |
| 62466 | 13:9078686 | 62545 | 13:10762211 | 62546 | 13:10778845 | 62548 | 13:10867741 | 62554 | 13:11145952 |
| 62555 | 13:11222783 | 62556 | 13:11320371 | 62557 | 13:11410445 | 62570 | 13:12019870 | 62634 | 13:13812138 |
| 62650 | 13:14311743 | 62692 | 13:15465789 | 62712 | 13:16020130 | 62799 | 13:17568011 | 62801 | 13:17588165 |
| 62835 | 13:18353118 | 63019 | 13:21321597 | 63087 | 13:23067175 | 63093 | 13:23318050 | 63105 | 13:23791918 |
| 63109 | 13:23900966 | 63124 | 13:24343896 | 63189 | 13:25699872 | 63323 | 13:29049594 | 63392 | 13:30294381 |
| 63393 | 13:30377425 | 63476 | 13:31944092 | 63477 | 13:32039794 | 63555 | 13:33385230 | 63576 | 13:34100138 |
| 63577 | 13:34193250 | 63578 | 13:34248535 | 63581 | 13:34427463 | 63706 | 13:37210289 | 63805 | 13:39259881 |
| 63838 | 13:39814181 | 64020 | 13:43484075 | 64302 | 13:48427057 | 64315 | 13:49071500 | 64316 | 13:49168510 |
| 64345 | 13:49747887 | 64346 | 13:49844262 | 64355 | 13:50115519 | 64357 | 13:50180207 | 64358 | 13:50268295 |
| 64372 | 13:50739564 | 64374 | 13:50832795 | 64381 | 13:51125057 | 64401 | 13:51523719 | 64402 | 13:51622569 |
| 64500 | 13:53510732 | 64501 | 13:53607473 | 64507 | 13:53822879 | 64519 | 13:54442428 | 64570 | 13:55504923 |
| 64652 | 13:57623027 | 64676 | 13:58277988 | 64741 | 13:59446084 | 64816 | 13:61419709 | 64828 | 13:61686658 |
| 64836 | 13:61962725 | 64928 | 13:63467047 | 64929 | 13:63559780 | 64949 | 13:64081941 | 65039 | 13:65929411 |
| 65137 | 13:68477424 | 65180 | 13:69587905 | 65181 | 13:69666536 | 65541 | 13:77055890 | 65542 | 13:77153724 |
| 65879 | 14:93414 | 65895 | 14:609870 | 65904 | 14:947222 | 66104 | 14:4750888 | 66610 | 14:13439017 |
| 66611 | 14:13473101 | 66612 | 14:13553001 | 66613 | 14:13632606 | 66614 | 14:13721229 | 66615 | 14:13734707 |
| 66616 | 14:13832328 | 66786 | 14:17490496 | 66822 | 14:18129262 | 66823 | 14:18227619 | 66827 | 14:18277849 |
| 66828 | 14:18376690 | 66874 | 14:19576484 | 67559 | 14:25819002 | 67636 | 14:26880974 | 67665 | 14:27799331 |
| 67800 | 14:30971186 | 67869 | 14:32582584 | 67898 | 14:33242218 | 67971 | 14:34837449 | 67972 | 14:34885142 |
| 67977 | 14:35171625 | 68004 | 14:35658219 | 68094 | 14:38119848 | 68123 | 14:38980550 | 68157 | 14:40059476 |
| 68173 | 14:40339877 | 68272 | 14:43115419 | 68466 | 14:47686908 | 68471 | 14:47920443 | 68533 | 14:49442868 |
| 68573 | 14:50805531 | 68645 | 14:52567142 | 68650 | 14:52653514 | 68657 | 14:52955563 | 68658 | 14:53054564 |
| 68676 | 14:53669390 | 68747 | 14:54918170 | 68781 | 14:55712044 | 68782 | 14:55807243 | 68783 | 14:55875273 |
| 68784 | 14:55953452 | 68824 | 14:57218378 | 68935 | 14:59606995 | 68952 | 14:60129379 | 68980 | 14:60666003 |
| 68981 | 14:60764297 | 69127 | 14:63797690 | 69169 | 14:64906621 | 69173 | 14:65094231 | 69187 | 14:65545962 |
| 69327 | 14:68518147 | 69328 | 14:68615752 | 69461 | 14:72182566 | 69496 | 14:72949159 | 69497 | 14:72993679 |
| 69514 | 14:73377613 | 69551 | 14:74402579 | 69585 | 14:75284663 | 69654 | 14:76712535 | 69927 | 15:278408 |
| 69928 | 15:364919 | 69935 | 15:581520 | 69938 | 15:790710 | 69940 | 15:921595 | 70011 | 15:2907387 |
| 70072 | 15:4118742 | 70298 | 15:5868982 | 70344 | 15:7132196 | 70468 | 15:9855083 | 70479 | 15:10238469 |
| 70485 | 15:10450890 | 70487 | 15:10557012 | 70500 | 15:11034094 | 70513 | 15:11544670 | 70515 | 15:11646233 |
| 70535 | 15:12337840 | 70536 | 15:12426564 | 70558 | 15:13317083 | 70564 | 15:13540062 | 70607 | 15:14553709 |
| 70611 | 15:14685598 | 70652 | 15:15637037 | 70768 | 15:17775660 | 70799 | 15:18549454 | 70821 | 15:19204706 |
| 70830 | 15:19463500 | 71142 | 15:25686598 | 71348 | 15:29961188 | 71383 | 15:30812283 | 71652 | 15:36545261 |
| 71720 | 15:37976104 | 71736 | 15:38441326 | 71771 | 15:39152334 | 71772 | 15:39251802 | 71782 | 15:39467016 |
| 72064 | 15:44893395 | 72102 | 15:45703046 | 72116 | 15:46016293 | 72117 | 15:46052010 | 72118 | 15:46128678 |
| 72120 | 15:46190128 | 72193 | 15:47315722 | 72195 | 15:47377732 | 72196 | 15:47473423 | 72199 | 15:47598007 |
| 72204 | 15:47732907 | 72264 | 15:48782827 | 72293 | 15:49903002 | 72297 | 15:50131403 | 72298 | 15:50228681 |
| 72321 | 15:50928742 | 72446 | 15:53225186 | 72482 | 15:53576274 | 72512 | 15:54128239 | 72554 | 15:55076548 |
| 72599 | 15:56354181 | 72600 | 15:56425647 | 72782 | 15:59421385 | 72830 | 15:60713798 | 72933 | 15:62959638 |
| 73204 | 15:68655064 | 73251 | 15:70097809 | 73252 | 15:70187975 | 73651 | 15:78826872 | 73652 | 15:78926080 |
| 73661 | 15:79238876 | 73664 | 15:79380785 | 73669 | 15:79544197 | 73670 | 15:79611716 | 73785 | 15:82130501 |
| 73795 | 15:82404865 | 73796 | 15:82425557 | 73797 | 15:82522759 | 73884 | 15:84032423 | 73887 | 15:84244168 |
| 73888 | 15:84260381 | 73889 | 15:84356903 | 73890 | 15:84447429 | 73891 | 15:84540077 | 73894 | 15:84686045 |
| 73895 | 15:84729195 | 73896 | 15:84804908 | 73897 | 15:84896615 | 73898 | 15:84939269 | 73901 | 16:338529 |
| 74061 | 16:3596934 | 74075 | 16:3906912 | 74153 | 16:5495502 | 74156 | 16:5663689 | 74159 | 16:5838621 |
| 74167 | 16:6306111 | 74197 | 16:6971134 | 74198 | 16:7015739 | 74199 | 16:7111115 | 74201 | 16:7234019 |
| 74221 | 16:7834741 | 74224 | 16:7996266 | 74228 | 16:8149785 | 74229 | 16:8194291 | 74256 | 16:9122788 |
| 74257 | 16:9217866 | 74265 | 16:9575503 | 74293 | 16:10558307 | 74409 | 16:13263254 | 74425 | 16:13790388 |
| 74496 | 16:15729876 | 74548 | 16:17488453 | 74550 | 16:17617156 | 74566 | 16:18152045 | 74575 | 16:18477012 |
| 74632 | 16:19928511 | 74709 | 16:21880848 | 74750 | 16:22924908 | 74816 | 16:24474002 | 74979 | 16:27452942 |
| 75007 | 16:28071735 | 75037 | 16:29095796 | 75043 | 16:29327042 | 75168 | 16:32723586 | 75199 | 16:33327718 |
| 75372 | 16:37660699 | 75428 | 16:38784562 | 75449 | 16:39690828 | 75490 | 16:40983811 | 75550 | 16:42163425 |
| 75555 | 16:42310324 | 75556 | 16:42386477 | 75560 | 16:42697916 | 75568 | 16:42993444 | 75580 | 16:43487631 |
| 75582 | 16:43668103 | 75584 | 16:43759140 | 75585 | 16:43845132 | 75586 | 16:43944562 | 75588 | 16:44032421 |
| 75608 | 16:44623635 | 75612 | 16:44826869 | 75613 | 16:44926372 | 75614 | 16:45026368 | 75616 | 16:45103219 |
| 75627 | 16:45512990 | 75632 | 16:45776402 | 75637 | 16:46002437 | 75779 | 16:49103242 | 75849 | 16:51054365 |
| 75850 | 16:51151596 | 75859 | 16:51446273 | 75867 | 16:51646308 | 75868 | 16:51740534 | 75944 | 16:53407745 |
| 75997 | 16:54730701 | 76093 | 16:56989855 | 76409 | 16:64087780 | 76503 | 16:65762070 | 76697 | 16:69928868 |
| 76757 | 16:71345017 | 76835 | 16:72851827 | 77085 | 16:77598464 | 77222 | 16:79857865 | 77229 | 16:80072357 |
| 77230 | 16:80138235 | 77232 | 16:80311616 | 77233 | 16:80396660 | 77235 | 16:80531024 | 77236 | 16:80630465 |
| 77239 | 16:80799166 | 77242 | 16:80953675 | 77252 | 17:259333 | 77263 | 17:564310 | 77274 | 17:973466 |
| 77278 | 17:1054010 | 77487 | 17:5568436 | 77620 | 17:8228129 | 77681 | 17:9733872 | 77735 | 17:10897059 |
| 77736 | 17:10938025 | 77737 | 17:11020572 | 77738 | 17:11094236 | 77755 | 17:11494094 | 77830 | 17:13277982 |
| 78070 | 17:18481630 | 78136 | 17:19808398 | 78137 | 17:19897663 | 78207 | 17:21967677 | 78230 | 17:22786786 |
| 78249 | 17:23511334 | 78255 | 17:23800711 | 78322 | 17:25936343 | 78401 | 17:27754738 | 78430 | 17:28426405 |
| 78441 | 17:28791960 | 78462 | 17:29447559 | 78487 | 17:30217269 | 78545 | 17:31620341 | 78567 | 17:32254712 |
| 78570 | 17:32296225 | 78667 | 17:34617927 | 78688 | 17:35304907 | 78690 | 17:35390256 | 78721 | 17:36229501 |
| 78734 | 17:36837955 | 78744 | 17:37134508 | 78795 | 17:38760200 | 78814 | 17:39443909 | 78936 | 17:41933427 |
| 78937 | 17:42031727 | 78986 | 17:42956052 | 78987 | 17:43050836 | 79027 | 17:44108368 | 79108 | 17:46082443 |
| 79178 | 17:47678534 | 79293 | 17:50425399 | 79393 | 17:52219542 | 79410 | 17:52792109 | 79481 | 17:54629610 |
| 79511 | 17:55298739 | 79535 | 17:55992314 | 79545 | 17:56246007 | 79980 | 17:64743637 | 80106 | 17:67144833 |
| 80130 | 17:67822583 | 80279 | 17:70683155 | 80280 | 17:70709345 | 80281 | 17:70709461 | 80291 | 17:71094967 |
| 80383 | 17:72831459 | 80395 | 18:95447 | 80396 | 18:189110 | 80398 | 18:367703 | 80400 | 18:514106 |
| 80908 | 18:9808096 | 80994 | 18:11474377 | 81022 | 18:12273578 | 81074 | 18:13692264 | 81098 | 18:14306814 |
| 81099 | 18:14400626 | 81103 | 18:14549787 | 81105 | 18:14605010 | 81106 | 18:14636355 | 81107 | 18:14731173 |
| 81108 | 18:14801380 | 81151 | 18:15872181 | 81153 | 18:15986314 | 81159 | 18:16182872 | 81377 | 18:20732386 |
| 81736 | 18:28718247 | 81811 | 18:30779424 | 81813 | 18:30921028 | 81837 | 18:31639943 | 81954 | 18:34144938 |
| 82045 | 18:36256440 | 82052 | 18:36555965 | 82152 | 18:38719188 | 82233 | 18:40460799 | 82285 | 18:41570886 |
| 82432 | 18:44652242 | 82443 | 18:44987987 | 82600 | 18:47190873 | 82671 | 18:48450267 | 82706 | 18:49271447 |
| 82707 | 18:49370683 | 82738 | 18:50083459 | 82759 | 18:50626419 | 82760 | 18:50725650 | 82837 | 18:52240322 |
| 82862 | 18:53044750 | 82869 | 18:53322848 | 82997 | 18:55515927 | 83109 | 18:56926638 | 83227 | 18:58451447 |
| 83237 | 18:58961238 | 83240 | 18:59153638 | 83249 | 18:59423487 | 83250 | 18:59502600 | 83251 | 18:59597971 |
| 83252 | 18:59640740 | 83253 | 18:59738484 | 83254 | 18:59752260 | 83255 | 18:59841292 | 83271 | 18:60187977 |
| 83273 | 18:60349514 | 83293 | 18:61084174 | 83294 | 18:61180054 | 83301 | 18:61392112 | 83302 | 18:61484398 |
| 83373 | 18:62547161 | 83374 | 18:62548072 | 83375 | 18:62548590 | 83376 | 18:62614389 | 83386 | 18:63006555 |
| 83387 | 18:63077361 | 83422 | 18:63662692 | 83588 | 19:57925 | 83591 | 19:305379 | 83596 | 19:417884 |
| 83597 | 19:509378 | 83599 | 19:577296 | 83600 | 19:668256 | 83629 | 19:1655405 | 83656 | 19:2558450 |
| 83665 | 19:2953866 | 83671 | 19:3120033 | 83677 | 19:3275802 | 83714 | 19:4397168 | 83980 | 19:9821822 |
| 84056 | 19:11385109 | 84253 | 19:15470245 | 84549 | 19:20561916 | 84554 | 19:20740986 | 84572 | 19:21295269 |
| 84638 | 19:22476147 | 84674 | 19:23274892 | 84679 | 19:23407987 | 84688 | 19:23703085 | 84976 | 19:27081319 |
| 84977 | 19:27125583 | 84978 | 19:27182483 | 84979 | 19:27275569 | 84986 | 19:27665926 | 85115 | 19:30350931 |
| 85176 | 19:31793582 | 85191 | 19:32153081 | 85511 | 19:37954850 | 85578 | 19:39940442 | 85649 | 19:41359770 |
| 85749 | 19:43284000 | 85860 | 19:45535788 | 85910 | 19:46199205 | 85919 | 19:46495194 | 85936 | 19:46990600 |
| 86031 | 19:48855388 | 86072 | 19:49706256 | 86111 | 19:50303597 | 86113 | 19:50431879 | 86114 | 19:50531654 |
| 86241 | 19:52131137 | 86250 | 19:52418467 | 86565 | 19:56677337 | 86621 | 19:57762258 | 86622 | 19:57849833 |
| 86961 | 20:98572 | 86962 | 20:194921 | 87107 | 20:2918386 | 87430 | 20:5583730 | 87593 | 20:8760244 |
| 87840 | 20:14544398 | 87842 | 20:14662366 | 87845 | 20:14842827 | 87846 | 20:14894317 | 87848 | 20:15043520 |
| 87850 | 20:15190994 | 87888 | 20:16233299 | 87915 | 20:16830331 | 87920 | 20:17017002 | 87956 | 20:18017776 |
| 87971 | 20:18426404 | 88037 | 20:20313521 | 88073 | 20:21317972 | 88231 | 20:23439914 | 88321 | 20:25028667 |
| 88322 | 20:25092607 | 88327 | 20:25369162 | 88338 | 20:25827366 | 88357 | 20:26444773 | 88371 | 20:26712661 |
| 88493 | 20:29517107 | 88527 | 20:30497850 | 88541 | 20:31003971 | 88598 | 20:32503045 | 88599 | 20:32522681 |
| 88600 | 20:32622352 | 88602 | 20:32778020 | 88695 | 20:34788751 | 88842 | 20:38032426 | 88884 | 20:39213757 |
| 89063 | 20:43010466 | 89065 | 20:43140540 | 89068 | 20:43254253 | 89073 | 20:43352423 | 89105 | 20:44220967 |
| 89127 | 20:44701792 | 89143 | 20:45276596 | 89180 | 20:46098985 | 89181 | 20:46185509 | 89205 | 20:47109652 |
| 89218 | 20:47365415 | 89224 | 20:47651501 | 89232 | 20:47954545 | 89237 | 20:48129247 | 89238 | 20:48218836 |
| 89257 | 20:48716255 | 89281 | 20:49326846 | 89283 | 20:49443154 | 89319 | 20:50486303 | 89360 | 20:51882151 |
| 89375 | 20:52355899 | 89376 | 20:52454269 | 89400 | 20:53180123 | 89438 | 20:54174787 | 89439 | 20:54272031 |
| 89441 | 20:54375180 | 89450 | 20:54601936 | 89464 | 20:55005757 | 89492 | 20:55642905 | 89496 | 20:55834687 |
| 89710 | 20:60274323 | 89746 | 20:61511139 | 89881 | 20:64307221 | 90177 | 20:70744162 | 90271 | 20:71587457 |
| 90278 | 20:71850045 | 90283 | 21:134737 | 90284 | 21:233193 | 90286 | 21:309646 | 90287 | 21:371966 |
| 90288 | 21:471326 | 90289 | 21:506222 | 90291 | 21:638478 | 90292 | 21:726326 | 90293 | 21:823686 |
| 90294 | 21:904065 | 90295 | 21:966923 | 90296 | 21:1061609 | 90297 | 21:1093204 | 90298 | 21:1137598 |
| 90300 | 21:1255004 | 90301 | 21:1331973 | 90302 | 21:1425976 | 90303 | 21:1485463 | 90304 | 21:1581245 |
| 90306 | 21:1694504 | 90308 | 21:1850823 | 90309 | 21:1924655 | 90323 | 21:2437601 | 90347 | 21:3138288 |
| 90349 | 21:3228959 | 90358 | 21:3544839 | 90359 | 21:3610217 | 90364 | 21:3820824 | 90406 | 21:4613245 |
| 90484 | 21:7029661 | 90640 | 21:10179845 | 91036 | 21:19148704 | 91170 | 21:21513912 | 91265 | 21:23382552 |
| 91266 | 21:23474228 | 91273 | 21:23715679 | 91285 | 21:24111132 | 91308 | 21:24835888 | 91334 | 21:25749419 |
| 91346 | 21:26098112 | 91360 | 21:26431821 | 91386 | 21:27005517 | 91387 | 21:27103067 | 91433 | 21:28660586 |
| 91552 | 21:31691093 | 91568 | 21:32209086 | 91593 | 21:32883760 | 91594 | 21:32927345 | 91595 | 21:33019785 |
| 91724 | 21:34773624 | 91725 | 21:34862867 | 91834 | 21:37259953 | 91836 | 21:37337560 | 91837 | 21:37418367 |
| 91858 | 21:38022062 | 91859 | 21:38118482 | 91861 | 21:38269475 | 91894 | 21:39232808 | 91946 | 21:40644524 |
| 91947 | 21:40743815 | 91953 | 21:40912381 | 91994 | 21:41910649 | 92018 | 21:42440438 | 92023 | 21:42514187 |
| 92102 | 21:44357068 | 92140 | 21:44936824 | 92141 | 21:45020642 | 92143 | 21:45198066 | 92152 | 21:45602636 |
| 92165 | 21:46016604 | 92166 | 21:46114894 | 92169 | 21:46249176 | 92297 | 21:48618715 | 92405 | 21:51567065 |
| 92449 | 21:52692952 | 92450 | 21:52792942 | 92453 | 21:52960457 | 92620 | 21:56228179 | 92621 | 21:56313681 |
| 92623 | 21:56441767 | 92814 | 21:59309517 | 92815 | 21:59375264 | 93006 | 21:63376882 | 93229 | 21:67773042 |
| 93338 | 22:94579 | 93352 | 22:646933 | 93358 | 22:824901 | 93437 | 22:2467585 | 93677 | 22:7210800 |
| 93714 | 22:8230735 | 93734 | 22:8758516 | 93765 | 22:9634144 | 93849 | 22:11365346 | 93942 | 22:13343219 |
| 93966 | 22:14036315 | 93997 | 22:14787281 | 94092 | 22:17058957 | 94135 | 22:18197695 | 94162 | 22:18799270 |
| 94176 | 22:19116049 | 94364 | 22:24074772 | 94470 | 22:27069723 | 94481 | 22:27482704 | 94563 | 22:29220441 |
| 94595 | 22:30012007 | 94650 | 22:31491877 | 94657 | 22:31704814 | 95074 | 22:41624457 | 95174 | 22:43984117 |
| 95228 | 22:45235116 | 95236 | 22:45525829 | 95393 | 22:48902067 | 95902 | 22:58335431 | 95953 | 22:59355819 |
| 96035 | 23:278582 | 96037 | 23:430451 | 96042 | 23:626975 | 96071 | 23:1548151 | 96072 | 23:1635007 |
| 96178 | 23:3824609 | 96286 | 23:5931144 | 96463 | 23:9588241 | 96613 | 23:12692439 | 96648 | 23:13322760 |
| 96655 | 23:13471246 | 96656 | 23:13531584 | 97061 | 23:21612939 | 97062 | 23:21712441 | 97063 | 23:21811769 |
| 97086 | 23:22310386 | 97103 | 23:22816656 | 97117 | 23:23369505 | 97336 | 23:25965936 | 97337 | 23:26013871 |
| 97338 | 23:26203805 | 97339 | 23:26302825 | 97340 | 23:26400435 | 97341 | 23:26404617 | 97342 | 23:26417922 |
| 97343 | 23:26516930 | 97346 | 23:26688460 | 97347 | 23:26750001 | 97348 | 23:26836934 | 97638 | 23:30204175 |
| 97639 | 23:30205302 | 97640 | 23:30205793 | 97675 | 23:30951436 | 97705 | 23:31625189 | 97851 | 23:34829880 |
| 97852 | 23:34888702 | 97859 | 23:35057295 | 97860 | 23:35151436 | 97889 | 23:36152667 | 97963 | 23:38043115 |
| 98233 | 23:42608340 | 98516 | 23:48087560 | 98519 | 23:48274159 | 98668 | 23:50824134 | 98767 | 23:52402327 |
| 98771 | 24:201134 | 98932 | 24:3597084 | 98987 | 24:4929150 | 99002 | 24:5203768 | 99108 | 24:7965661 |
| 99109 | 24:8063828 | 99131 | 24:8583177 | 99304 | 24:10935542 | 99358 | 24:12240445 | 99425 | 24:13974458 |
| 99478 | 24:15604573 | 99486 | 24:15989036 | 99487 | 24:16079054 | 99561 | 24:18229831 | 99572 | 24:18541933 |
| 99573 | 24:18640412 | 99606 | 24:19585603 | 99618 | 24:19942905 | 99624 | 24:20173407 | 99688 | 24:21131115 |
| 99764 | 24:22214747 | 99765 | 24:22312329 | 99816 | 24:23615391 | 99822 | 24:23975968 | 99826 | 24:24145037 |
| 99942 | 24:26860326 | 10005 | 24:29671134 | 10010 | 24:30883521 | 10010 | 24:30966522 | 10021 | 24:33423144 |
| 100223 | 24:33544520 | 10033 | 24:36290089 | 10034 | 24:36532147 | 10039 | 24:38166045 | 10040 | 24:38422210 |
| 100411 | 24:38722958 | 10047 | 24:40089318 | 10047 | 24:40188306 | 10054 | 24:41297881 | 10062 | 24:42939236 |
| 100630 | 24:43395685 | 10064 | 24:43924798 | 10064 | 24:44019504 | 10079 | 24:46654829 | 10085 | 24:48033977 |
| 100863 | 24:48144296 | 10087 | 24:48567652 | 10088 | 24:48858324 | 10101 | 24:52201248 | 10105 | 24:52902393 |
| 101118 | 24:54423570 | 10118 | 24:55890705 | 10120 | 24:56201929 | 10149 | 24:61932447 | 10150 | 24:62180714 |
| 101504 | 24:62277921 | 10150 | 25:94971 | 10161 | 25:2097812 | 10174 | 25:3066063 | 10199 | 25:7620341 |
| 102020 | 25:8255600 | 10204 | 25:8952928 | 10205 | 25:9079254 | 10228 | 25:14296254 | 10246 | 25:18216204 |
| 102662 | 25:22863656 | 10269 | 25:23717883 | 10285 | 25:26775859 | 10304 | 25:30451801 | 10304 | 25:30672143 |
| 103152 | 25:32745193 | 10360 | 25:40515689 | 10370 | 26:71749 | 10370 | 26:134754 | 10370 | 26:202644 |
| 103710 | 26:292215 | 10371 | 26:332815 | 10371 | 26:418885 | 10371 | 26:467022 | 10371 | 26:552651 |
| 103715 | 26:648778 | 10372 | 26:841636 | 10372 | 26:933342 | 10374 | 26:1410312 | 10376 | 26:2231655 |
| 103788 | 26:2771137 | 10388 | 26:4804367 | 10390 | 26:5316048 | 10391 | 26:5782436 | 10402 | 26:8899248 |
| 104047 | 26:9422315 | 10405 | 26:9629233 | 10413 | 26:11253125 | 10413 | 26:11345317 | 10413 | 26:11428247 |
| 104158 | 26:11886123 | 10426 | 26:14376252 | 10428 | 26:15092211 | 10428 | 26:15180609 | 10432 | 26:16278407 |
| 104330 | 26:16409629 | 10433 | 26:16616504 | 10448 | 26:19837144 | 10449 | 26:20063209 | 10450 | 26:20330629 |
| 104526 | 26:21059250 | 10461 | 26:23492556 | 10462 | 26:23837564 | 10463 | 26:23968846 | 10481 | 26:27340392 |
| 104865 | 26:28704657 | 10492 | 26:30296052 | 10493 | 26:30555507 | 10496 | 26:31506273 | 10510 | 26:34494728 |
| 105234 | 26:37659896 | 10530 | 26:39301430 | 10532 | 26:39903212 | 10538 | 26:41736203 | 10542 | 26:42498087 |
| 105426 | 26:42598014 | 10545 | 26:43157974 | 10552 | 26:44983809 | 10558 | 26:45965280 | 10575 | 26:49691984 |
| 105779 | 26:50189494 | 10578 | 26:50197785 | 10578 | 26:50203962 | 10578 | 26:50261536 | 10578 | 26:50350367 |
| 105784 | 26:50350409 | 10578 | 26:50414892 | 10585 | 27:99932 | 10585 | 27:181641 | 10585 | 27:250836 |
| 105858 | 27:316731 | 10585 | 27:394123 | 10586 | 27:443903 | 10586 | 27:543122 | 10586 | 27:642666 |
| 105867 | 27:739223 | 10586 | 27:838134 | 10586 | 27:928860 | 10587 | 27:1021674 | 10587 | 27:1119724 |
| 105923 | 27:1865958 | 10599 | 27:3535767 | 10599 | 27:3755867 | 10611 | 27:6215443 | 10611 | 27:6337782 |
| 106121 | 27:6434403 | 10612 | 27:6434442 | 10612 | 27:6621177 | 10612 | 27:6714941 | 10612 | 27:6754631 |
| 106126 | 27:6761335 | 10612 | 27:6952355 | 10612 | 27:6962879 | 10612 | 27:6980835 | 10613 | 27:6994129 |
| 106250 | 27:9301634 | 10626 | 27:9835607 | 10627 | 27:10239566 | 10632 | 27:11805253 | 10633 | 27:12112133 |
| 106347 | 27:12322130 | 10634 | 27:12386638 | 10645 | 27:14207575 | 10665 | 27:18065646 | 10666 | 27:18151157 |
| 106710 | 27:19373108 | 10676 | 27:20532211 | 10677 | 27:20833561 | 10682 | 27:22102480 | 10682 | 27:22221678 |
| 106829 | 27:22350322 | 10684 | 27:22785446 | 10699 | 27:26185311 | 10711 | 27:28416757 | 10718 | 27:30124180 |
| 107366 | 27:33574896 | 10737 | 27:34012998 | 10747 | 27:35694726 | 10753 | 27:36991890 | 10762 | 27:38609999 |
| 107654 | 27:39285504 | 10789 | 27:44800138 | 10791 | 27:45569924 | 10792 | 28:39225 | 10792 | 28:138659 |
| 107922 | 28:231989 | 10792 | 28:285448 | 10792 | 28:328039 | 10792 | 28:386583 | 10792 | 28:563052 |
| 107930 | 28:662354 | 10793 | 28:794289 | 10793 | 28:893452 | 10801 | 28:2643580 | 10802 | 28:2904232 |
| 108027 | 28:2999388 | 10803 | 28:3161546 | 10803 | 28:3253315 | 10803 | 28:3405359 | 10804 | 28:3759573 |
| 108043 | 28:3787485 | 10804 | 28:3885321 | 10804 | 28:4073833 | 10804 | 28:4163374 | 10805 | 28:4253300 |
| 108051 | 28:4338728 | 10805 | 28:4513285 | 10806 | 28:5004251 | 10818 | 28:7591484 | 10827 | 28:9724722 |
| 108313 | 28:10660380 | 10832 | 28:11111391 | 10832 | 28:11222161 | 10843 | 28:13814734 | 10854 | 28:16313184 |
| 108613 | 28:17683321 | 10863 | 28:18306254 | 10874 | 28:21104115 | 10877 | 28:22035988 | 10879 | 28:22784794 |
| 108801 | 28:22871308 | 10880 | 28:22889009 | 10884 | 28:23719431 | 10884 | 28:23801384 | 10902 | 28:27215263 |
| 109182 | 28:30404928 | 10933 | 28:33475488 | 10938 | 28:34421752 | 10943 | 28:35588714 | 10949 | 28:36727428 |
| 109624 | 28:39439768 | 10962 | 28:39494932 | 10963 | 28:39750515 | 10993 | 28:44515897 | 11000 | 29:178851 |
| 110009 | 29:265250 | 11001 | 29:607202 | 11007 | 29:1956188 | 11008 | 29:2091012 | 11023 | 29:5374583 |
| 110238 | 29:5456512 | 11023 | 29:5547355 | 11024 | 29:5627071 | 11024 | 29:5698810 | 11028 | 29:7011954 |
| 110333 | 29:8334191 | 11033 | 29:8529992 | 11054 | 29:13277373 | 11055 | 29:13442800 | 11058 | 29:14334295 |
| 110592 | 29:14552146 | 11066 | 29:16276745 | 11073 | 29:18298255 | 11074 | 29:18821433 | 11074 | 29:19176738 |
| 110756 | 29:19577395 | 11081 | 29:20938903 | 11081 | 29:21062969 | 11084 | 29:21741949 | 11102 | 29:26286827 |
| 111040 | 29:26637610 | 11104 | 29:26812835 | 11105 | 29:27123234 | 11105 | 29:27286001 | 11106 | 29:27336935 |
| 111061 | 29:27426724 | 11138 | 29:34145956 | 11151 | 29:36681488 | 11156 | 29:37610625 | 11157 | 29:37814400 |
| 111573 | 29:37903323 | 11157 | 29:37975002 | 11157 | 29:38065526 | 11157 | 29:38161198 | 11157 | 29:38251275 |
| 111578 | 29:38348064 | 11157 | 29:38414602 | 11158 | 29:38450767 | 11158 | 29:38467768 | 11158 | 29:38556318 |
| 111583 | 29:38649308 | 11158 | 29:38752213 | 11158 | 29:38847684 | 11158 | 29:38917900 | 11158 | 29:38952660 |
| 111590 | 29:38993981 | 11159 | 29:39083787 | 11159 | 29:39205805 | 11172 | 29:41919049 | 11214 | 29:50420995 |
| 112179 | 29:50977672 | 11218 | 29:51071432 | | | | | | |
| . | | | | | | | | | |
